(19) United States
(12) Patent Application Publication
(10) Pub. No.: US 2026/0345786 A1
(43) Pub. Date: Nov. 5, 2026

(54) Novel Nitrogen-Containing Heterocyclic Compounds

(71) Applicant: Pfizer Inc., New York, NY (US)

(72) Inventors: Chulho Choi, Mystic, CT (US); Andrew Fensome, Harvard, MA (US); Kentaro Futatsugi, Sharon, MA (US); Brian Stephen Gerstenberger, Boston, MA (US); Mohammad Mehdi Ghahremanpour, Manchester, NH (US); David Hepworth, Concord, MA (US); Kevin Michael Knopf, Groton, CT (US); Daniel Wei-Shung Kung, Salem, CT (US); Fang-Yu Lin, Needham, MA (US); Ellene Hera Mashalidis, Salem, CT (US); Sarah Jane Mear, East Lyme, CT (US); Mihir Dineshkumar Parikh, East Greenwich, RI (US); Ian Brooks Perry, Pelham, NH (US); Daniel Jonathan Smaltz, Cambridge, MA (US); Jianmin Sun, East Lyme, CT (US); Benjamin Aaron Thuma, Old Lyme, CT (US); Tao Wang, Sudbury, MA (US); Liuqing Wei, Ledyard, CT (US); Ian William Windsor, Mystic, CT (US)

(73) Assignee: Pfizer Inc., New York, NY (US)

(21) Appl. No.: 19/644,519

(22) Filed: Apr. 10, 2026

Related U.S. Application Data
(60) Provisional application No. 63/787,492, filed on Apr. 11, 2025.
(60) Provisional application No. 63/826,151, filed on Jun. 18, 2025.
(60) Provisional application No. 63/826,142, filed on Jun. 18, 2025.
(60) Provisional application No. 63/889,197, filed on Sep. 26, 2025.
(60) Provisional application No. 63/894,419, filed on Oct. 6, 2025.
(60) Provisional application No. 63/900,521, filed on Oct. 16, 2025.
(60) Provisional application No. 63/942,386, filed on Dec. 16, 2025.

(51) Int. Cl. A61K 47/54

(52) U.S. Cl. CPC A61K 47/545

(57) ABSTRACT

The disclosure relates to compounds of the disclosure and pharmaceutically acceptable salts thereof to their use in medicine; to compositions containing them; to processes for their preparation; and to intermediates used in such processes. The compounds of the disclosure may be useful in the treatment, prevention, suppression and amelioration of disease(s) such as atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa. The compounds of the disclosure may be useful in the treatment, prevention, suppression and amelioration of a dermatological condition or a respiratory condition.

### BACKGROUND

The present disclosure relates to novel compounds. The disclosure also relates to the preparation of the compounds and intermediates used in the preparation, compositions containing the compounds, and uses of the compounds including their use as a STAT6 inhibitor.

STAT6 is a member of the Signal Transducer and Activator of Transcription (STAT) family of proteins consisting of transcription factors that impact cell processes including differentiation, survival, proliferation, and functional activation [Levy, D E and Darnell, J E. STATs: transcriptional control and biological impact. 2002. Nat Rev Mol Cell Biol. 3(9):651-62]. The STAT family consists of seven members: STAT1, STAT2, STAT3, STAT4, STAT5a, STAT5b, and STAT6.

STAT family proteins are downstream targets of the Janus kinase (JAK) family kinases, which contribute to signal transduction from a variety of cytokines including IL-2, IL-5, GM-CSF, IL-10, IL-12, IL-23, as well as IL-4 and IL-13. Cytokines IL-4 and IL-13 have been demonstrated to signal through STAT6 activation [Kaplan, M H et al. 1996. Stat6 is required for mediating responses to IL-4 and for development of Th2 cells. Immunity. 4: 313-319]. The pathogenic activity of IL-4 and IL-13 cytokines is consistent with efficacy that has been observed with JAK inhibitors, which block signaling of IL-4 and IL-13 as well as signaling of additional inflammatory cytokines [Simpson, E L et al. 2020. Efficacy and safety of abrocitinib in adults and adolescents with moderate-to-severe atopic dermatitis (JADE MONO-1): a multicentre, double-blind, randomised, placebo-controlled, phase 3 trial. Lancet. 396(10246): 255-266; Guttman-Yassky, E et al. 2021. Once-daily upadacitinib versus placebo in adolescents and adults with moderate-to-severe atopic dermatitis (Measure Up 1 and Measure Up 2): results from two replicate double-blind, randomised controlled phase 3 trials. Lancet. 397(10290): 2151-2168].

Targeted protein degradation (TPD) has been an emerging field that has attracted substantial attention. Instead of inhibiting a protein of interest (POI), protein degraders utilize a cellular system such as ubiquitin proteasome system (UPS) to induce selective protein degradation. A few protein degraders (e.g. fulvestrant) have been approved and more degraders including proteolysis targeting chimeras (PROTACs) and Molecular Glues are in clinical development (Bekes, M. et. al. Nat. Rev. Drug. Disc. 2022, 21, 181-200). There is no approved STAT6 degrader. University of Michigan reported a few STAT6 PROTAC degraders (Kaneshige, A. et. al. J. Med. Chem, 2024).

Despite the effectiveness of known therapeutics, an unmet need remains for safe and effective therapeutics for numerous diseases characterized by inflammatory responses, that address a broad range of pathogenic mechanisms.

### BRIEF SUMMARY

The present disclosure provides, in part, compounds and pharmaceutically acceptable salts thereof. Such compounds may inhibit the activity of STAT6 and may be useful in the treatment, prevention, suppression, and/or amelioration of disease(s), disorders and conditions mediated by STAT6. Also provided are pharmaceutical compositions, comprising the compounds or salts, alone or in combination with additional therapeutic agents. The present disclosure also provides, in part, methods for preparing such compounds, pharmaceutically acceptable salts and compositions of the disclosure, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

In an aspect, a compound of Formula I, or a pharmaceutically acceptable salt thereof:

M-T-Z I
wherein Z is a ubiquitin ligase binding moiety;
wherein T is a linker that connects Z to M;
wherein M is a targeting moiety having the formula M:

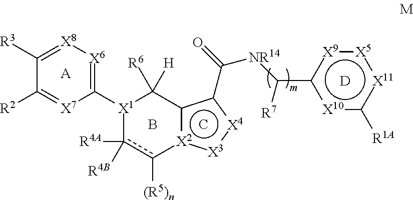

wherein the remaining variables are disclosed herein below.

In an aspect, the compound is of Formula I, or a pharmaceutically acceptable salt thereof,

M-T-Z (I)
wherein Z is a ubiquitin ligase binding moiety;
wherein T is a linker that connects Z to M; and
wherein M is a targeting moiety as defined herein that can be derived from any of Examples 1 to 758 or any of Examples E1A to E434A or any of prophetic examples V1 to V142 or any of prophetic examples V1A to V187A.

In an aspect, the compound of Formula I is any of Examples E1 to E732.

In an aspect, the disclosure relates to a pharmaceutical composition comprising a compound of the disclosure, or a pharmaceutically acceptable salt thereof, and at least one pharmaceutically acceptable excipient.

In another aspect, the disclosure relates to a method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprising administering to a subject in need thereof a therapeutically effective amount of the compound of the disclosure.

In another aspect, the disclosure relates to the compound of the disclosure or a pharmaceutically acceptable salt thereof for use as a medicament; or for use in the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa; or for use in the treatment of at least one of a dermatological condition or a respiratory condition.

It is to be understood that both the foregoing general description and the following detailed description are exemplary and explanatory only and are not restrictive of the disclosure, as claimed.

### DETAILED DESCRIPTION

The present disclosure may be understood more readily by reference to the following detailed description and the Examples included herein. It is to be understood that this disclosure is not limited to specific synthetic methods of making that may of course vary. It is to be also understood that the terminology used herein is for the purpose of describing specific aspects only and is not intended to be limiting.

In an aspect, a compound of Formula I, or a pharmaceutically acceptable salt thereof:

M-T-Z I
wherein Z is a ubiquitin ligase binding moiety;
wherein T is a linker that connects Z to M;
wherein M is a targeting moiety having the Formula M:

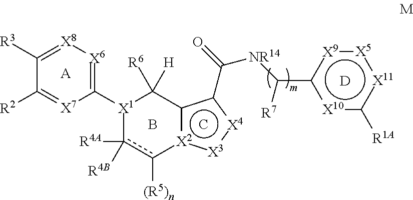

wherein
X¹ is CH, CNH₂, or N;
X² is C or N;
X³ is CR¹³, N, NR¹³, O, or S;
X⁴ is CH, N, or NR¹³;
X⁵ is CR^{1B} or N;
X⁶ is CH, CR¹⁵, or N;
X⁷ is CH, N, R^{2A}, or CF;
X⁸ is CH or N;
X⁹ is CR^{1B} or N;
X¹⁰ is CR^{1B} or N;
X¹¹ is CR¹, N, or NCH₃;
R¹ is —NR¹⁰—C(═O)—, —NR¹⁰—C(═O)—NR¹⁰—, H, —C(═O)—NR¹⁰—, —NHR⁸, —OH, —C₁₋₄ hydroxyalkyl, —C₁₋₃ fluoroalkyl, —C₀₋₁ alkylene-C₂₋₆ cyclyl, —C(═O)—NR¹⁰R⁸, —C(═O)—NR¹⁰—C₀₋₁ alkylene-phenyl, —C₀₋₁ alkylene-C₁₋₅ heterocyclyl, —O—C₁₋₅ heterocyclyl, or —C₁₋₅ alkyl; wherein the —C₁₋₅ heterocyclyl or the —C₂₋₆ cyclyl of R¹ is optionally substituted with one, two, or three of —C₁₋₃ alkyl, halogen, —OH, —C(═O)—N(R¹⁰)₂, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₅ alkyl of R¹ is optionally substituted with one or two of oxo, —NR^{10A}R¹¹, or —NR¹⁰R¹¹, a 5 to 6 membered cycloalkyl, a 5 to 6 membered heterocycloalkyl, cyano, fluoromethoxy, or methoxy; wherein the respective a 5 to 6 membered cycloalkyl and a 5 to 6 membered heterocycloalkyl groups of R¹ are optionally substituted with one or two of —OH, oxo, halogen, —C₁₋₃ alkoxy, —C₁₋₃ alkyl-C₁₋₃ alkoxy, or —C₁₋₃ alkyl;
R^{1A} is H, halogen, —C₂₋₃ heterocyclyl, oxo, —C(═O)NR¹⁰R¹¹; or —CH₃; wherein the —C₂₋₃ heterocyclyl is optionally substituted with —C₁₋₃ alkyl; or
R¹ and R^{1A} taken together form a C₄₋₇ heterocycloalkyl fused to Ring D or a C₄₋₇ heteroaryl fused to Ring D; wherein the C₄₋₇ heterocycloalkyl or the C₄₋₇ heteroaryl is optionally substituted with one or two oxo or —C₂₋₃ oxoalkyl;
each R^{1B} independently is H, —CH₃, F, Cl, or methoxy;
R² is H, —C₁₋₃ alkyl, —C₁₋₃ alkoxy, —C₁₋₃ fluoroalkyl, or halogen; or
R² and R^{2A} taken together form a six membered heteroaryl fused to Ring A;
R³ is —C(═O)—, —C(═O)—NR^{12A}—, —NR^{12A}—C(═O)—, —C₂₋₇ heterocyclylene-, —C₁₋₃ alkyl, —C₁₋₁₀ heterocyclyl, —P(═O)(CH₃)₂, —S(═O)CH₃, —NH—S(═O)₂CH₃, or —NH—C(═O)—C₁₋₃ alkyl; wherein the —C₁₋₃ alkyl of R³ is optionally substituted with one, two, or three of oxo, halogen, —C₃₋₇ cycloalkyl, —OH, —NR¹²R^{12A}, or cyano; wherein the —C₁₋₁₀ heterocyclyl is optionally substituted with one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo; or
R² and R³ taken together form a C₃₋₆ heterocyclyl fused to Ring A optionally substituted with one, two, or three of —C₁₋₃ alkyl, —C₂₋₃ oxoalkyl, or oxo;
R^{4A} is H, —OH, —C₁₋₃ fluoroalkyl, or —C₁₋₃ alkyl;
R^{4B} is H or is absent; or
R^{4A} and R^{4B} taken together form cyclopropyl;
each R⁵ independently is H, halogen, —OH, cyclopropyl, —C₁₋₃ fluoroalkyl, or —C₁₋₃ alkyl; or two R⁵ groups taken together form a C₃₋₄ cycloalkyl or a C₃₋₅ heterocycloalkyl;
R⁶ is H, —OH, cyano, or —C₁₋₃ alkyl; or
R^{4A} and R⁶ or one R⁵ and R⁶ taken together form a C₁₋₃ alkyl bridge or a C₁₋₃ heteroalkyl bridge; or
R^{4B} and one R⁵ taken together form a C₃₋₅ cycloalkyl fused to Ring B;
R⁷ is H, —C₁₋₃ alkyl, or —C₁₋₃ hydroxyalkyl; or
R⁸ is H, —C₁₋₃ alkyl, —SO₂CH₃, or a —C₂₋₅ heterocyclyl; wherein the —C₁₋₃ alkyl of R³ is optionally substituted with one, two, or three of oxo, —C₃₋₉ heterocyclyl, —C₁₋₃ alkoxy, cyanoimine, —C₅₋₆ aryl, —O—C₅₋₆ aryl, or —NR⁹R¹⁰; wherein the —C₂₋₅ heterocyclyl of R³ is optionally substituted with one, two, or three of oxo, halogen, —C₀₋₁ alkylene-NR¹⁰R¹¹, —OH, —C₁₋₃ hydroxyalkyl, C₁₋₃alkoxy optionally substituted with —NR¹⁰R¹¹, —C₁₋₃ alkyl-C₁₋₃ alkoxy, —C₁₋₃ oxoalkyl, or —C₁₋₃ alkyl; wherein the —C₃₋₉ heterocycloalkyl of R³ is optionally substituted with one, two, or three of —C₁₋₃ alkyl, —OH, —C₁₋₃ hydroxyalkyl, —O—C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O—C₃₋₅ heterocycloalkyl, phenyl, or —C₀₋₁ alkylene-NR¹⁰R¹¹; wherein the phenyl is optionally substituted with halogen; wherein the —C₅₋₆ aryl is optionally substituted with a C₄₋₅ heterocycloalkyl or halogen;
each R⁹ independently is H, —C₁₋₅ alkyl, —C₁₋₄ hydroxyalkyl, —C₁₋₃ fluoroalkyl, —C₃₋₅ cycloalkyl, —C₃₋₅ heterocycloalkyl, -methylene-phenylene-NH—C(═O)—NR¹⁰R¹¹, or —C₃₋₅ heterocycloalkyl; wherein the —C₁₋₅ alkyl of R⁹ is optionally substituted with one, two, or three of —NR¹⁰R¹¹, —C₃₋₅ cycloalkyl, —C₃₋₅ heterocycloalkyl, cyano, fluoromethoxy, or methoxy; wherein the respective —C₃₋₅ cycloalkyl and —C₃₋₅ heterocycloalkyl groups of R⁹ are optionally substituted with one or two of —OH, oxo, halogen, —C₁₋₃ alkoxy, —C₁₋₃ alkyl-C₁₋₃ alkoxy, or —C₁₋₃ alkyl;
each R¹⁰ independently is H or —C₁₋₄ alkyl; or
each R^{10A} independently is H, —C₁₋₅ alkyl, —C₁₋₄ hydroxyalkyl, —C₁₋₃ fluoroalkyl, —C₃₋₅ cycloalkyl, —C₃₋₅ heteroaryl, -methylene-phenylene-NH—C(═O)—NR¹⁰R¹¹, or —C₀₋₁ alkylene-C₃₋₅ heterocycloalkyl optionally substituted with —C₁₋₃ alkyl; wherein the —C₁₋₅ alkyl of R^{10A} is optionally substituted with one, two, or three of —NR¹⁰R¹¹, —C₃₋₅ cycloalkyl, —C₃₋₅ heterocycloalkyl, cyano, fluoromethoxy, or methoxy; wherein the respective —C₃₋₅ cycloalkyl and —C₃₋₅ heterocycloalkyl groups of R^{10A} are optionally substituted with one or two of —OH, oxo, halogen, —C₁₋₃ alkoxy, —C₁₋₃ alkyl-C₁₋₃ alkoxy, or —C₁₋₃ alkyl;
each R¹¹ independently is H or —C₁₋₃ alkyl;
R¹² is H, —C₁₋₃ alkyl, —C₁₋₃ hydroxyalkyl, —(CH₂)₀₋₂ alkylene-C₃₋₅ heterocycloalkyl, —(CH₂)₁₋₂ alkylene-O—C₃₋₅ heterocycloalkyl, -methylene-C(═O)—NR¹⁰R¹¹, or -methylene-phenylene-NH—C(═O)—NR¹⁰R¹¹; wherein the —(CH₂)₀₋₂ alkylene-C₃₋₅ heterocycloalkyl or the —(CH₂)₁₋₂ alkylene-O—C₃₋₅ heterocycloalkyl of R¹² is optionally substituted with —C₁₋₄ alkyl, —C₁₋₃ alkyl-C₁₋₃ alkoxy, —(CH₂)₀₋₁ alkylene-phenyl, halogen, —C₁₋₃ fluoroalkyl, or —(CH₂)₀₋₁ alkylene-C₃₋₅ heterocycloalkyl; wherein the —C₁₋₃ alkyl of R¹² is optionally substituted with one, two, or three of oxo or NR¹⁰R¹¹;
R^{12A} is H or —C₁₋₄ alkyl; or
R¹² and R^{12A} taken together form a C₃₋₁₀ heterocyclyl optionally substituted with one, two, or three of —OH, —C₁₋₃ alkyl, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR¹⁰R¹¹, or —NHC(═O)CH₃;
R¹³ is —C₁₋₆ alkylene-, —C(═O)—, H, —C₁₋₆ alkyl, —C₁₋₄ hydroxyalkyl, —C₁₋₃ fluoroalkyl, —C₁₋₃ alkylene-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃₋₅ heterocycloalkyl; wherein the —C₁₋₆ alkyl of R¹³ is optionally substituted with one, two, or three of oxo, fluoro, —NH₂, C₃₋₆ cycloalkyl, or methoxy; wherein the —C₀₋₁ alkylene-C₃₋₅ cycloalkyl or the —C₀₋₁ alkylene-C₃₋₆ heterocycloalkyl of R¹³ is optionally substituted with one, two, or three of —C₁₋₃ alkyl, —C₁₋₃ fluoroalkyl, or halogen;
R¹⁴ is H or —C₁₋₃ alkyl; or
X⁴ is NR¹³ and R¹³ and R¹⁴ taken together form a C₄₋₅ heterocyclyl fused to Ring C; or
X¹⁰ and R¹⁴ taken together form a 5 to 6-membered heterocyclyl that is optionally substituted with C₁₋₃ alkyl and that is fused to ring D;
n is 1 or 2;
m is 0 or 1;
wherein if X³ is CR¹³ or NR¹³ then X⁴ is CH or N, and if X⁴ is NR¹³ then X³ is N, O, or S;
wherein the respective heterocyclyl and heteroaryl each independently include 1 to 4 heteroatoms including at least one of N, O, P, or S; and
one of R¹, R³, R¹³, or R¹⁵ is optionally a bivalent connector that is connected to T.

In an aspect, a compound of Formula I, or a pharmaceutically acceptable salt thereof:

M-T-Z I
wherein Z is a ubiquitin ligase binding moiety;
wherein T is a linker that connects Z to M;
wherein M is a targeting moiety having the Formula M-1:

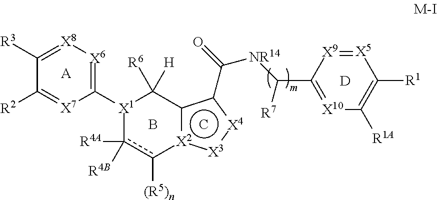

wherein
X¹ is CH, CNH₂, or N;
X² is C or N;
X³ is CR¹³, N, NR¹³, O, or S;
X⁴ is CH, N, or NR¹³;
X⁵ is CR^{1B} or N;
X⁶ is CH or N;
X⁷ is CH, N, R^{2A}, or CF;
X⁸ is CH or N;
X⁹ is CR^{1B} or N;
X¹⁰ is CR^{1B} or N;
R¹ is —NR¹⁰—C(═O)—, —NR¹⁰—C(═O)—NR¹⁰—, H, —C(═O)—NR¹⁰—, —NHR⁸, —OH, —C₂₋₅ heterocyclyl, or —C₁₋₃ alkyl; wherein the —C₂₋₅ heterocyclyl of R¹ is optionally substituted with one, two, or three of —C₁₋₃ alkyl, halogen, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₃ alkyl of R¹ is optionally substituted with one or two of oxo, —NR^{10A}R¹¹, or —NR¹⁰R¹¹;
R^{1A} is H, halogen, or —CH₃; or
R¹ and R^{1A} taken together form a C₅₋₇ heterocycloalkyl fused to Ring D or a C₅₋₇ heteroaryl fused to Ring D; wherein the C₅₋₇ heterocycloalkyl or the C₅₋₇ heteroaryl is optionally substituted with oxo;
each R^{1B} independently is H, —CH₃, F, C, or methoxy;
R² is H, —C₁₋₃ alkyl, —C₁₋₃ alkoxy, —C₁₋₃ fluoroalkyl, or halogen;
R² and R^{2A} taken together form a six membered heteroaryl fused to Ring A;
R³ is —C(═O)—, —C(═O)—NR^{12A}—, —NR^{12A}—C(═O)—, —C₂₋₅ heterocyclylene-, —C₁₋₃ alkyl, —C₁₋₁₀ heterocyclyl, —P(═O)(CH₃)₂, —S(═O)CH₃, —NH—S(═O)₂CH₃, or —NH—C(═O)—C₁₋₃ alkyl; wherein the —C₁₋₃ alkyl of R³ is optionally substituted with one, two, or three of oxo, halogen, —C₃₋₆ cycloalkyl, —OH, —NR¹²R^{12A}, or cyano; wherein the —C₁₋₁₀ heterocyclyl is optionally substituted with one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo; or
R² and R³ taken together form a C₃₋₆ heterocyclyl fused to Ring A optionally substituted with one, two, or three of —C₁₋₃ alkyl, —C₂₋₃ oxoalkyl, or oxo;
R^{4A} is H, —OH, —C₁₋₃ fluoroalkyl, or —C₁₋₃ alkyl;
R^{4B} is H or is absent; or
R^{4A} and R^{4B} taken together form cyclopropyl;
each R⁵ independently is H, halogen, —OH, cyclopropyl, —C₁₋₃ fluoroalkyl, or —C₁₋₃ alkyl; or two R⁵ groups taken together form a C₃₋₄ cycloalkyl or a C₃₋₅ heterocycloalkyl;
R⁶ is H, —OH, cyano, or —C₁₋₃ alkyl; or
R^{4A} and R⁶ or one R⁵ and R⁶ taken together form a C₁₋₃ alkyl bridge or a C₁₋₃ heteroalkyl bridge; or
R^{4B} and one R⁵ taken together form a C₃₋₅ cycloalkyl fused to Ring B;
R⁷ is H, —C₁₋₃ alkyl, or —C₁₋₃ hydroxyalkyl;
R⁸ is H, —C₁₋₃ alkyl, —SO₂CH₃, or a —C₂₋₅ heterocyclyl; wherein the —C₁₋₃ alkyl of R⁸ is optionally substituted with one, two, or three of oxo, —C₃₋₉ heterocyclyl, —C₁₋₃ alkoxy, cyanoimine, —O—C₅₋₆ aryl, or —NR⁹R¹⁰; wherein the —C₂₋₅ heterocyclyl of R⁸ is optionally substituted with one, two, or three of oxo, halogen, —C₀₋₁ alkylene-NR¹⁰R¹¹, —OH, —C₁₋₃ hydroxyalkyl, —C₁₋₃ alkoxy optionally substituted with —NR¹⁰R¹¹, —C₁₋₃ alkyl-C₁₋₃ alkoxy, —C₁₋₃ oxoalkyl, or —C₁₋₃ alkyl; wherein the —C₃₋₉ heterocycloalkyl of R⁸ is optionally substituted with one, two, or three of —C₁₋₃ alkyl, —OH, —C₁₋₃ hydroxyalkyl, —O—C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O—C₃₋₅ heterocycloalkyl, phenyl, or —C₀₋₁ alkylene-NR¹⁰R¹¹; wherein the phenyl is optionally substituted with halogen;
R⁹ is H, —C₁₋₅ alkyl, —C₁₋₄ hydroxyalkyl, —C₁₋₃ fluoroalkyl, —C₃₋₅ cycloalkyl, —C₃₋₅ heterocycloalkyl, -methylene-phenylene-NH—C(═O)—NR¹⁰R¹¹, or —C₃₋₅ heterocycloalkyl; wherein the —C₁₋₄ alkyl of R⁹ is optionally substituted with one, two, or three of —NR¹⁰R¹¹, —C₃₋₅ cycloalkyl, —C₃₋₅ heterocycloalkyl, cyano, fluoromethoxy, or methoxy; wherein the respective —C₃₋₅ cycloalkyl and —C₃₋₅ heterocycloalkyl groups of R⁹ are optionally substituted with one or two of —OH, oxo, halogen, —C₁₋₃ alkoxy, —C₁₋₃ alkyl-C₁₋₃ alkoxy, or —C₁₋₃ alkyl;
each R¹⁰ independently is H or —C₁₋₄ alkyl; or
R^{10A} is H, —C₁₋₅ alkyl, —C₁₋₄ hydroxyalkyl, —C₁₋₃ fluoroalkyl, —C₃₋₅ cycloalkyl, —C₃₋₅ heterocycloalkyl, -methylene-phenylene-NH—C(═O)—NR¹⁰R¹¹, —C₃₋₅ heterocycloalkyl, or —C₀₋₁ alkylene-C₃₋₅ heterocycloalkyl optionally substituted with —C₁₋₃ alkyl; wherein the —C₁₋₄ alkyl of R^{10A} is optionally substituted with one, two, or three of —NR¹⁰R¹¹, —C₃₋₅ cycloalkyl, —C₃₋₅ heterocycloalkyl, cyano, fluoromethoxy, or methoxy; wherein the respective —C₃₋₅ cycloalkyl and —C₃₋₅ heterocycloalkyl groups of R^{10A} are optionally substituted with one or two of —OH, oxo, halogen, —C₁₋₃ alkoxy, —C₁₋₃ alkyl-C₁₋₃ alkoxy, or —C₁₋₃ alkyl;
each R¹¹ independently is H or —C₁₋₃ alkyl;
R¹² is H, —C₁₋₃ alkyl, —C₁₋₃ hydroxyalkyl, —(CH₂)₀₋₂ alkylene-C₃₋₅ heterocycloalkyl, —(CH₂)₁₋₂ alkylene-O—C₃₋₅ heterocycloalkyl, -methylene-C(═O)—NR¹⁰R¹¹, or -methylene-phenylene-NH—C(═O)—NR¹⁰R¹¹; wherein the —(CH₂)₀₋₂ alkylene-C₃₋₅ heterocycloalkyl or the —(CH₂)₁₋₂ alkylene-O—C₃₋₅ heterocycloalkyl of R¹² is optionally substituted with —C₁₋₄ alkyl, —C₁₋₃ alkyl-C₁₋₃ alkoxy, —(CH₂)₀₋₁ alkylene-phenyl, halogen, —C₁₋₃ fluoroalkyl, or —(CH₂)₀₋₁ alkylene-C₃₋₅ heterocycloalkyl; wherein the —C₁₋₃ alkyl of R¹² is optionally substituted with one, two, or three of oxo or NR¹⁰R¹¹;
R^{12A} is H or —C₁₋₄ alkyl; or
R¹² and R^{12A} taken together form a C₃₋₁₀ heterocyclyl optionally substituted with one, two, or three of —OH, —C₁₋₃ alkyl, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR¹⁰R¹¹, or —NHC(═O)CH₃;
R¹³ is —C₁₋₆ alkylene-, —C(═O)—, H, —C₁₋₆ alkyl, —C₁₋₄ hydroxyalkyl, —C₁₋₃ fluoroalkyl, —C₁₋₃ alkylene-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃₋₅ heterocycloalkyl; wherein the —C₁₋₆ alkyl of R¹³ is optionally substituted with one, two, or three of oxo, fluoro, —NH₂, C₃₋₆ cycloalkyl, or methoxy; wherein the —C₀₋₁ alkylene-C₃₋₅ cycloalkyl or the —C₀₋₁ alkylene-C₃₋₆ heterocycloalkyl of R¹³ is optionally substituted with one, two, or three of —C₁₋₃ alkyl, —C₁₋₃ fluoroalkyl, or halogen;
R¹⁴ is H; or
X⁴ is NR¹³ and R¹³ and R¹⁴ taken together form a C₄₋₅ heterocyclyl fused to Ring C; or
X¹⁰ and R¹⁴ taken together form a 5 to 6-membered heterocyclyl that is optionally substituted with C₁₋₃ alkyl and that is fused to ring D;
n is 1 or 2;
m is 0 or 1;
wherein if X³ is CR¹³ or NR¹³ then X⁴ is CH or N, and if X⁴ is NR¹³ then X³ is N, O, or S;
wherein the respective heterocyclyl and heteroaryl each independently include 1 to 4 heteroatoms including at least one of N, O, P, or S; and
one of R¹, R³, R¹³, or R¹⁵ can be a bivalent connector that is connected to T.

In an aspect, a compound of Formula I, or a pharmaceutically acceptable salt thereof:

M-T-Z I
wherein Z is a ubiquitin ligase binding moiety;
wherein T is a linker that connects Z to M;
wherein M is a targeting moiety having the Formula M:

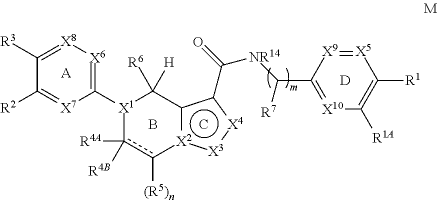

wherein
X¹ is CH, CNH₂, or N;
X² is C or N;
X³ is CR¹³, N, NR¹³, O, or S;
X⁴ is CH, N, or NR¹³;
X⁵ is CR^{1B} or N;
X⁶ is CH or N;
X⁷ is CH, N, or CF;
X⁸ is CH or N;
X⁹ is CR^{1B} or N;
X¹⁰ is CR^{1B} or N;
R¹ is —NR¹⁰—C(═O)—, —NR¹⁰—C(═O)—NR¹⁰—, H, —C(═O)—NR¹⁰—, —NHR⁸, —OH, —C₂₋₅ heterocyclyl, or —C₁₋₃ alkyl; wherein the —C₂₋₅ heterocyclyl of R¹ is optionally substituted with one, two, or three of —C₁₋₃ alkyl, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₃ alkyl of R¹ is optionally substituted with one or two of oxo, —NR^{10A}R¹¹, or —NR¹⁰R¹¹;
R^{1A} is H, halogen, or —CH₃; or
R¹ and R^{1A} taken together form a C₅₋₇ heterocycloalkyl fused to Ring D or a C₅₋₇ heteroaryl fused to Ring D; wherein the C₅₋₇ heterocycloalkyl or the C₅₋₇ heteroaryl is optionally substituted with oxo;
each R^{1B} independently is H, —CH₃, F, Cl, or methoxy;
R² is H, —C₁₋₃ alkyl, —C₁₋₃ alkoxy, —C₁₋₃ fluoroalkyl, or halogen;
R³ is —C(═O)—, —C(═O)—NR^{12A}—, —NR^{12A}—C(═O)—, —C₁₋₃ alkyl, —C₂₋₁₀ heterocyclyl, —P(═O)(CH₃)₂, —S(═O)CH₃, —NH—S(═O)₂CH₃, or —NH—C(═O)—C₁₋₃ alkyl; wherein the —C₁₋₃ alkyl of R³ is optionally substituted with one, two, or three of oxo, halogen, —C₃₋₆ cycloalkyl, —OH, —NR¹²R^{12A}, or cyano; wherein the —C₂₋₁₀ heterocyclyl is optionally substituted with one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo; or
R² and R³ taken together form a C₃₋₆ heterocyclyl fused to Ring A optionally substituted with one, two, or three of —C₁₋₃ alkyl, —C₂₋₃ oxoalkyl, or oxo;
R^{4A} is H, —OH, —C₁₋₃ fluoroalkyl, or —C₁₋₃ alkyl;
R^{4B} is H or is absent; or
R^{4A} and R^{4B} taken together form cyclopropyl;
each R⁵ independently is H, halogen, —OH, cyclopropyl, —C₁₋₃ fluoroalkyl, or —C₁₋₃ alkyl; or two R⁵ groups taken together form cyclopropyl;
R⁶ is H, —OH, cyano, or —C₁₋₃ alkyl; or
R^{4A} and R⁶ or one R⁵ and R⁶ taken together form a C₁₋₃ alkyl bridge or a C₁₋₃ heteroalkyl bridge; or
R^{4B} and one R⁵ taken together form a C₃₋₅ cycloalkyl fused to Ring B;
R⁷ is H, —C₁₋₃ alkyl, or —C₁₋₃ hydroxyalkyl;
R³ is H, —C₁₋₃ alkyl, —SO₂CH₃, or a —C₃₋₄ heterocyclyl; wherein the —C₁₋₃ alkyl of R³ is optionally substituted with one, two, or three of oxo, —C₃₋₉ heterocycloalkyl, —C₁₋₃ alkoxy, cyanoimine, or —NR⁹R¹⁰; wherein the —C₃₋₄ heterocyclyl of R³ is optionally substituted with one, two, or three of oxo, halogen, —C₀₋₁ alkylene-NR¹⁰R¹¹, —OH, —C₁₋₃ hydroxyalkyl, —C₁₋₃ alkoxy optionally substituted with —NR¹⁰R¹¹, —C₁₋₃ alkyl-C₁₋₃ alkoxy, —C₁₋₃ oxoalkyl, or —C₁₋₃ alkyl; wherein the —C₃₋₉ heterocycloalkyl of R³ is optionally substituted with one, two, or three of —C₁₋₃ alkyl, —OH, —C₁₋₃ hydroxyalkyl, —O—C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O—C₃₋₅ heterocycloalkyl, or —C₀₋₁ alkylene-NR¹⁰R¹¹;
R⁹ is H, —C₁₋₄ alkyl, —C₁₋₄ hydroxyalkyl, —C₁₋₃ fluoroalkyl, —C₃₋₅ cycloalkyl, -methylene-phenylene-NH—C(═O)—NR¹⁰R¹¹, or —C₃₋₅ heterocycloalkyl; wherein the —C₁₋₄ alkyl of R⁹ is optionally substituted with one, two, or three of —C₃₋₅ cycloalkyl, —C₃₋₅ heterocycloalkyl, or methoxy; wherein the —C₃₋₅ cycloalkyl of R⁹ is optionally substituted with one or two of —OH or —C₁₋₃ alkyl;
each R¹⁰ independently is H or —C₁₋₄ alkyl; or
R^{10A} is —C₀₋₁ alkylene-C₃₋₅ heterocycloalkyl optionally substituted with —C₁₋₃ alkyl;
each R¹¹ independently is H or —C₁₋₃ alkyl;
R¹² is H, —C₁₋₃ alkyl, —C₁₋₃ hydroxyalkyl, —(CH₂)₀₋₂ alkylene-C₃₋₅ heterocycloalkyl, —(CH₂)₁₋₂ alkylene-O—C₃₋₅ heterocycloalkyl, -methylene-C(═O)—NR¹⁰R¹¹, or -methylene-phenylene-NH—C(═O)—NR¹⁰R¹¹; wherein the —(CH₂)₀₋₂ alkylene-C₃₋₅ heterocycloalkyl or the —(CH₂)₁₋₂ alkylene-O—C₃₋₅ heterocycloalkyl of R¹² is optionally substituted with —C₁₋₄ alkyl, —C₁₋₃ alkyl-C₁₋₃ alkoxy, —(CH₂)₀₋₁ alkylene-phenyl, halogen, —C₁₋₃ fluoroalkyl, or —(CH₂)₀₋₁ alkylene-C₃₋₅ heterocycloalkyl; wherein the —C₁₋₃ alkyl of R¹² is optionally substituted with one, two, or three of oxo or NR¹⁰R¹¹;
R^{12A} is H or —C₁₋₄ alkyl; or
R¹² and R^{12A} taken together form a C₃₋₁₀ heterocyclyl optionally substituted with one, two, or three of —OH, —C₁₋₃ alkyl, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR¹⁰R¹¹, or —NHC(═O)CH₃;
R¹³ is —C₁₋₆ alkylene-, —C(═O)—, H, —C₁₋₆ alkyl, —C₁₋₄ hydroxyalkyl, —C₁₋₃ fluoroalkyl, —C₁₋₃ alkylene-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃₋₅ heterocycloalkyl; wherein the —C₁₋₆ alkyl of R¹³ is optionally substituted with one, two, or three of oxo, fluoro, —NH₂, C₃₋₆ cycloalkyl, or methoxy; wherein the —C₃₋₅ cycloalkyl or the —C₃₋₆ cycloalkyl of R¹³ is optionally substituted with one, two, or three of —C₁₋₃ alkyl, —C₁₋₃ fluoroalkyl, or halogen;
R¹⁴ is H; or
X⁴ is NR¹³ and R¹³ and R¹⁴ taken together form a C₄₋₅ heterocyclyl fused to Ring C;
n is 1 or 2;
m is 0 or 1;
wherein if X³ is CR¹³ or NR¹³ then X⁴ is CH or N, and if X⁴ is NR¹³ then X³ is N, O, or S;
wherein the respective heterocyclyl and heteroaryl each independently include 1 to 4 heteroatoms including at least one of N, O, or S; and
with the proviso that one of R¹, R³, or R¹³ is a bivalent connector and is connected to T.

In an aspect, a compound of Formula I, or a pharmaceutically acceptable salt thereof:

M-T-Z I
wherein Z is a ubiquitin ligase binding moiety;
wherein T is a linker that connects Z to M;
wherein M is a targeting moiety derived from any one of wherein M is a targeting moiety derived from any of Examples 1 to 758 or any of Examples E1A to E434A or any of prophetic examples V1 to V142 or any of prophetic examples V1A to V187A; wherein M is bonded to T via a bond at the corresponding R¹, R³, R¹³, or R¹⁵ locations as illustrated in Formula M or M-1.

In an aspect, R¹ is the bivalent connector, and the compound has the Formula IA:

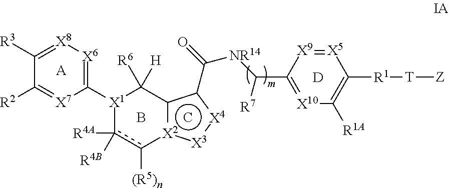

wherein R¹ is —NH—C(═O)—, —NH—C(═O)—NR¹⁰— or —C(═O)—NR¹⁰—.

In an aspect, at least one of a) X³ is NR¹³ and R¹³ is C₁₋₃ alkyl or C₁₋₃ fluoroalkyl or b) R³ is —C(═O)—NR¹²R^{12A} or —C₂₋₁₀ heterocyclyl; and wherein the —C₂₋₁₀ heterocyclyl is optionally substituted with one, two, or three of —C₁₋₃ alkyl, —C₁₋₃ fluoroalkyl, or oxo.

In an aspect, R³ is the bivalent connector, and the compound has the Formula IB:

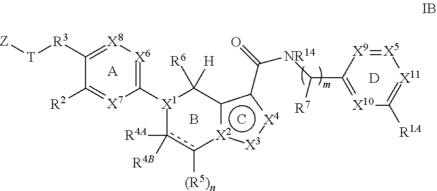

wherein R³ is —C₂₋₅ heterocyclyl-, —C(═O)—, —C(═O)—NR^{12A}— or —NR^{12A}—C(═O)—; wherein X¹¹ is optionally CR¹.

In an aspect, at least one of a) X³ is NR¹³ and b) R¹³ is C₁₋₃ alkyl or C₁₋₃ fluoroalkyl; or R¹ is —C(═O)—NHR^{10A} or —NH—C(═O)—NR⁹R¹⁰.

In an aspect, X¹, X², and X⁴ are N; X³ is CR¹³; R¹³ is C₁₋₃ alkyl or C₁₋₃ fluoroalkyl.

In an aspect, R¹³ is the bivalent connector, and the compound has the Formula IC or Formula ID:

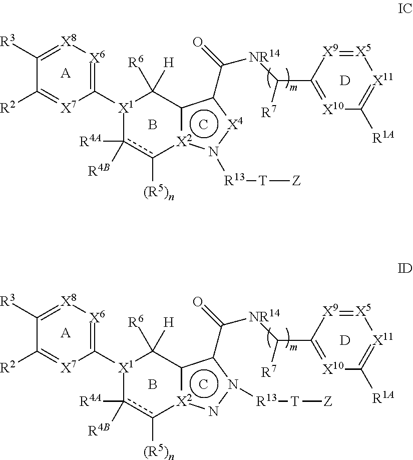

wherein R¹³ is —C₁₋₆ alkylene- or —C(═O)—; wherein X¹¹ is optionally CR¹.

In an aspect, at least one of a) R¹ is —NH—C(═O)—NR⁹R¹⁰ or b) R³ is —C(═O)—NR¹²R^{12A} or —C₂₋₁₀ heterocyclyl; wherein the —C₂₋₁₀ heterocyclyl is optionally substituted with one, two, or three of —C₁₋₃ alkyl.

In an aspect, R¹⁵ is the bivalent connector, and the compound has the Formula IE:

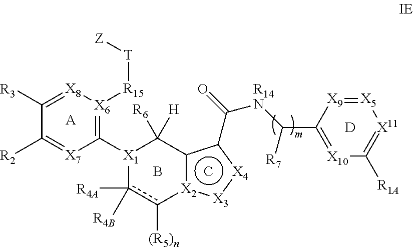

wherein R¹⁵ is a direct bond, —O—, —C₁₋₆ alkylene-, or —C(═O)—.

In an aspect, T has the formula -T¹-, -T¹-T²-, -T¹-T³-, -T²-T³-, or -T¹-T²-T³- or wherein T has the formula -T¹-, -T¹-T²-, -T¹-T³-, or -T¹-T²-T³-;
T¹ is —C(═O)—, —C₁₋₆ alkoxy-, —C₁₋₆ fluoroalkoxy-, —C₁₋₆ alkylene-, —C₁₋₆ fluoroalkylene-, —C₀₋₃ alkylene-C₅₋₆ arylene-, —C₀₋₃ alkylene-C₄₋₅ heteroarylene-, —C₀₋₃ alkylene-C₃₋₁₀ cycloalkylene-C₀₋₃ alkylene, —C₀₋₃ alkylene-C₃₋₉ heterocycloalkylene-C₀₋₃ alkylene-, —C₀₋₃ alkylene-O—C₃₋₉ heterocycloalkylene-C₀₋₃ alkylene-, —C₁₋₃ alkylene-C≡C—, —C₁₋₃ alkylene-, or —C₁₋₃ alkylene-O—; wherein the C₃₋₈ heterocycloalkylene groups of T¹ have 1, 2, or 3 heteroatoms selected from O, N, and S; and wherein T¹ is optionally substituted with one, two, or three of T⁴; and;
T² is —O—, —NH—C(═O)-T⁸-, —S(═O)₂-T⁸-, —O—S(O)₂-T⁸-, —C(═O)—, —NT⁷-, —C₃₋₆ cycloalkylene-, —C₃₋₆ heterocycloalkylene-, —C₁₋₆ alkylene-, —C₁₋₆ alkylene-O—, —C₁₋₆ aminoalkylene-, —C₁₋₆ fluoroalkylene-, —C₁₋₆ alkoxy-, —C₁₋₆ fluoroalkoxy-, —C₅₋₆ arylene, or —C₄₋₅ heteroarylene-; wherein T² is optionally substituted with one, two, or three T⁵; and wherein the respective heteroarylene or heterocycloalkylene groups each independently have 1, 2, or 3 heteroatoms selected from O, N, and S;
T³ is —C₁₋₆ alkoxy-, —C(═O)—, —C₁₋₆ alkylene-, —C₁₋₆ aminoalkylene-, —NH—C₁₋₆ alkylene-, —C(═O)—C₁₋₆ alkylene-, —C₁₋₆ alkylene-C≡C—, —C₁₋₃ alkylene-O—, —C₀₋₆ alkylene-C₃₋₈ heterocycloalkylene-C₀₋₆ alkylene-, —C₀₋₆ alkylene-O—C₃₋₈ heterocycloalkylene-C₀₋₆ alkylene-, —C₀₋₆ alkylene-C₃₋₈ heterocycloalkylene-C(═O)—, —C₅₋₆ arylene-, —C₃₋₈ heteroalkylene-C₁₋₆ alkylene-C₅₋₆ arylene-C₁₋₆ alkoxy-, —C₁₋₆ heteroalkylene-C₁₋₆ heterocycloalkylene-C(═O)—, —O—C₁₋₆ heterocycloalkylene-C(═O)—, —C₁₋₆ cycloalkylene-C₁₋₆ heterocycloalkylene-, —C₀₋₂ alkylene-C₅₋₆ arylene-C₁₋₆ alkoxy-, or —C₄₋₅ heteroarylene-; wherein T³ is optionally substituted with one, two, or three T⁶; wherein the respective -heteroarylene- or -heterocycloalkylene- groups have 1, 2, or 3 heteroatoms selected from O, N, and S;
T⁴, T⁵, and T⁶ are each independently oxo, —OH, halogen, —C₁₋₆ alkyl, —C₁₋₆ alkoxy, —C₁₋₆ fluoroalkyl, —C₃₋₆ cycloalkyl, —C₅₋₆ heteroaryl, or —C₃₋₆ heterocyclyl, wherein the heteroaryl and the heterocyclyl each independently have 1, 2, or 3 heteroatoms selected from 0 and N;
T⁷ is H or C₁₋₆ alkyl; and
T⁸ is —C₁₋₆ alkyl, —C₁₋₆ alkoxy, —C₁₋₆ fluoroalkyl, or —C₃₋₆ cycloalkyl;
or a pharmaceutically acceptable salt thereof.

In an aspect, T has the formula -T¹-, -T¹-T²-, -T¹-T³-, or -T¹-T²-T³; wherein T¹ is —C₀₋₃ alkylene-C≡C—, —C₁₋₆ alkoxy-, —C₁₋₆ alkylene-, —C₀₋₃ alkylene-C₃₋₉ heterocyclylene-, —C₀₋₃ alkylene-C₅₋₆ arylene-; for example, piperidinylene or piperazinylene; wherein the —C₃₋₉ heterocyclylene- is optionally substituted with halogen or —OH; wherein T² is —O—, —NR¹⁰—, —C₃₋₆ cycloalkylene-, —C₀₋₃ alkylene-C₅s arylene-, or —C₁₋₆ alkylene-O—; wherein if T² is —O—, then T³ is not —C₀ alkylene-O—C₃₋₈ heterocycloalkylene-C₀₋₆ alkylene- or —C₀ alkylene-NR¹⁰—; wherein T³ is —C₁₋₆ alkoxy-, —C₁₋₆ alkylene-, —C₀₋₃ alkylene-C≡C—C₀₋₃ alkylene-, —C₁₋₃ alkylene-O—, —C₁₋₃ alkylene-O—C₁₋₃ alkylene-C(═O)—C₃₋₈ heterocyclylene-, —C₁₋₃ alkylene-O—C₁₋₃ alkylene-C(═O)—NH—, —C₀₋₆ alkylene-NR¹⁰—C₀₋₆ alkylene-, —C₀₋₆ alkylene-C₃₋₈ heterocyclylene-C₀₋₆ alkylene-, or —C₀₋₆ alkylene-O—C₃₋₈ heterocyclylene-C₀₋₆ alkylene-; wherein the respective C₃₋₈ heterocyclylenes are optionally substituted with 1 or 2 halogen; or a pharmaceutically acceptable salt thereof.

In an aspect, T¹ is —C₁₋₃ alkylene-, —C₀₋₃ alkylene-C₃₋₈ heterocyclylene-, or —C₀₋₃ alkylene-C₅₋₆ arylene-; for example, piperidinylene or piperazinylene; wherein the —C₃₋₅ heterocyclylene- is optionally substituted with halogen or —OH; or a pharmaceutically acceptable salt thereof.

In an aspect, T¹ is —C₁₋₆ alkoxy-, —C₁₋₆ alkylene-, —C₀₋₃ alkylene-C₃₋₉ heterocycloalkylene-, for example, piperidinylene or piperazinylene; or a pharmaceutically acceptable salt thereof.

In an aspect, T² is —O—, —NR¹⁰—, —C₃₋₆ cycloalkylene-, or —C₀₋₃ alkylene-C₅s arylene-; wherein if T² is —O—, then T³ is not —C₀ alkylene-O—C₃₋₈ heterocycloalkylene-C₀₋₆ alkylene- or —C₀ alkylene-NR¹⁰—; or a pharmaceutically acceptable salt thereof.

In an aspect, T² is —O—, —C₃₋₆ cycloalkylene-, —C₅₋₆ arylene, —C₁₋₆ alkylene-, or —C₁₋₆ alkylene-O—; or a pharmaceutically acceptable salt thereof.

In an aspect, T³ is —C₁₋₃ alkylene-, —C₁₋₃ alkylene-C≡C—, —C≡C—C₁₋₃ alkylene-, —C₁₋₃ alkylene-O—, —C₁₋₃ alkylene-O—C₁₋₃ alkylene-C(═O)—C₃₋₈ heterocyclylene-, —C₁₋₃ alkylene-O—C₁₋₃ alkylene-C(═O)—NH—, —C₀₋₆ alkylene-NR¹⁰—C₀₋₃ alkylene-, —C₀₋₃ alkylene-C₃₋₈ heterocyclylene-C₀₋₃ alkylene-, or —C₀₋₃ alkylene-O—C₃₋₈ heterocyclylene-C₀₋₃ alkylene-; wherein the respective C₃₋₈ heterocyclylenes include 1 to 3 heteroatoms and are optionally substituted with 1 or 2 halogen (ex. E7); or a pharmaceutically acceptable salt thereof.

In an aspect, T³ is —C₁₋₆ alkoxy-, —C₁₋₆ alkylene, —C₁₋₆ alkylene-C≡C—, —C₁₋₃ alkylene-O—, —C₀₋₆ alkylene-C₃₋₈ heterocycloalkylene-C₀₋₆ alkylene-, or —C₀₋₆ alkylene-O—C₃₋₈ heterocycloalkylene-C₀₋₆ alkylene-; or a pharmaceutically acceptable salt thereof.

In an aspect, wherein T has the formula -T¹-, -T¹-T²-, -T¹-T³-, or -T¹-T²-T³;
T¹ is —C₀₋₅ alkylene-C₃₋₁₀ heterocycloalkylene-;
T² is —O—, or —C₀₋₆ alkylene-NR¹⁰—;
T³ is —C₁₋₆ alkylene-, —C₁₋₆ alkylene-C≡C—, —C₀₋₆ alkylene-C₃₋₉ heterocycloalkylene-C₀₋₆ alkylene-, —C₀₋₆ alkylene-C₃₋₉ heterocycloalkylene-C₁₋₆ alkylene-O—, —C₀₋₆ alkylene-C₃₋₉ cycloclylene-C₀₋₆ alkylene-, —C₀₋₆ alkylene-C₃₋₉ cycloclylene-C₁₋₆ alkylene-O—;
or a pharmaceutically acceptable salt thereof.

In an aspect, T has the formula -T¹-, -T¹-T²-, -T¹-T³-, -T²-T³-, or -T¹-T²-T³-;
T¹ is —C₀₋₅ alkylene-C₃₋₁₀ heterocycloalkylene-;
T² is —O—, —C₁₋₆ alkylene-, —C₁₋₆ alkylene-O—, or —C₀₋₆ alkylene-NR¹⁰—;
T³ is —C₁₋₆ alkylene-, —C₁₋₆ alkylene-C≡C—, —C₀₋₆ alkylene-C₃₋₉ heterocycloalkylene-C₀₋₆ alkylene-, —C₀₋₆ alkylene-C₃₋₉ heterocycloalkylene-C₁₋₆ alkylene-O—, —C₀₋₆ alkylene-C₃₋₉ cycloclylene-C₀₋₆ alkylene-, —C₀₋₆ alkylene-C₃₋₉ cycloclylene-C₁₋₆ alkylene-O—;
or a pharmaceutically acceptable salt thereof.

In an aspect, Z is present and has the Formula ZI:

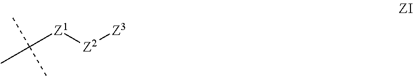

Z¹ is —C₃₋₁₄ heterocycloalkylene-, —C₁₋₁₀ heteroarylene-, or —C₆₋₁₀ arylene-; wherein is optionally substituted with one or two or three of halogen, —C₁₋₆ fluoroalkyl, —C₁₋₆ alkyl, C₁₋₃ alkoxy, or oxo; wherein the heteroarylene or heterocycloalkylene groups of Z¹ each independently include 1, 2, or 3 heteroatoms selected from O, N, and S;
Z² is a covalent bond, —NH—, —O—, —NCH₃—, —C(═O)—NH—, —CH₂—, or —C₂₋₃ alkylene-, wherein 1 of the methylene units of the —C₂₋₃ alkylene- is optionally replaced with —O—, —C(═O)—, —C(═S)—, —C(R^{1Z})₂—,
Z³ is

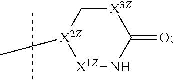

X^{1Z} is a covalent bond, CH₂, C(═O), CHCF₃, SO₂, S(═O), P(═O)C₁₋₃ alkyl, P(═O)OC₁₋₃ alkyl, P(═O)N(C₁₋₃ alkyl)₂, C(═S), or C—C₃₋₄ heteroaryl; wherein the heteroaryl of X^{1Z} includes 1, 2, or 3 heteroatoms selected from O, N, and S;
X^{2Z} is CH, N, or SiH;
X^{3Z} is CH₂, NH, O, S, or Si(C₁₋₃ alkyl)₂;
each R^{1Z} independently is H, —C₁₋₆ alkyl, phenyl, or a —C₃₋₅ heterocyclyl; wherein the —C₃₋₅ heterocyclyl of R^{1Z} has 1 to 4 heteroatoms selected from N, O, and S;
or a pharmaceutically acceptable salt thereof.

In an aspect, Z¹ is —C₆₋₈ heteroarylene- or —C₅₋₆ arylene-; wherein Z¹ is optionally substituted with one or two or three of halogen, —C₁₋₃ fluoroalkyl, —C₁₋₃ alkyl, C₁₋₃ alkoxy, or oxo; wherein the heteroarylene or heterocycloalkylene groups of Z¹ each independently include 1, 2, or 3 heteroatoms of N; or a pharmaceutically acceptable salt thereof.

In an aspect, Z² is the covalent bond, —NH—, or —CH₂— or a pharmaceutically acceptable salt thereof.

In an aspect; X^{1Z} is C(═O), X^{2Z} is CH or N, and X^{3Z} is CH₂; or a pharmaceutically acceptable salt thereof.

In an aspect, Z² is the covalent bond and wherein Z³ has the Formula Z^{3-b}

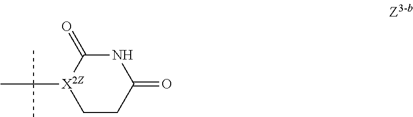

or a pharmaceutically acceptable salt thereof.

In an aspect, Z is one of the following:

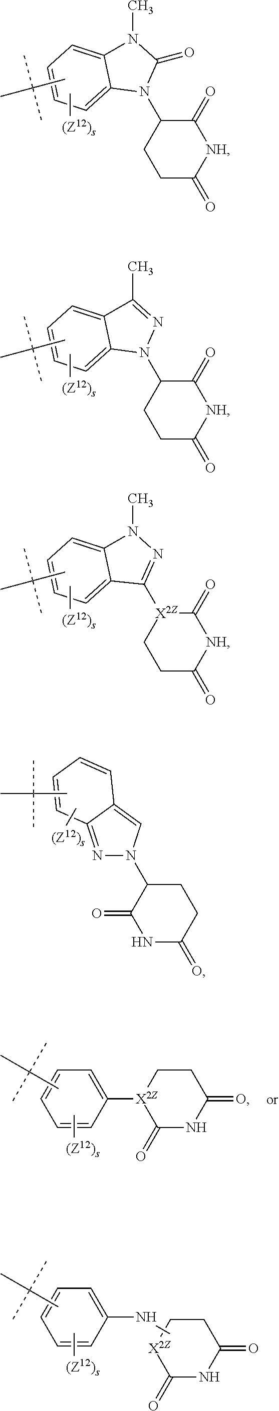

wherein Z¹² is halogen, C₁₋₃ alkoxy, or C₁₋₃ alkyl; and
wherein s is 0 or 1;
or a pharmaceutically acceptable salt thereof.

In an aspect, Z is one of the following:

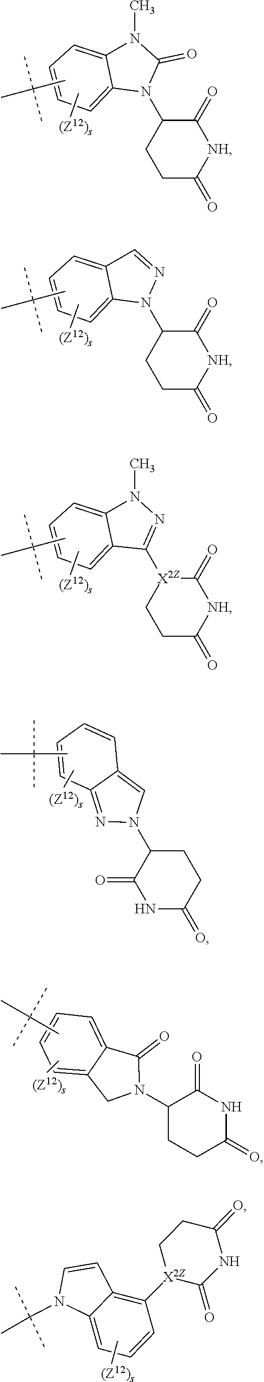

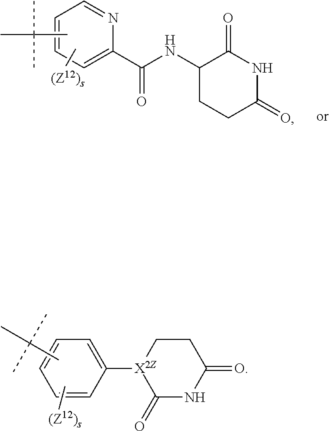

wherein Z¹² is halogen, C₁₋₃ alkoxy, or C₁₋₃ alkyl; and
wherein s is 0 or 1;

In an aspect, Z is one of the following:

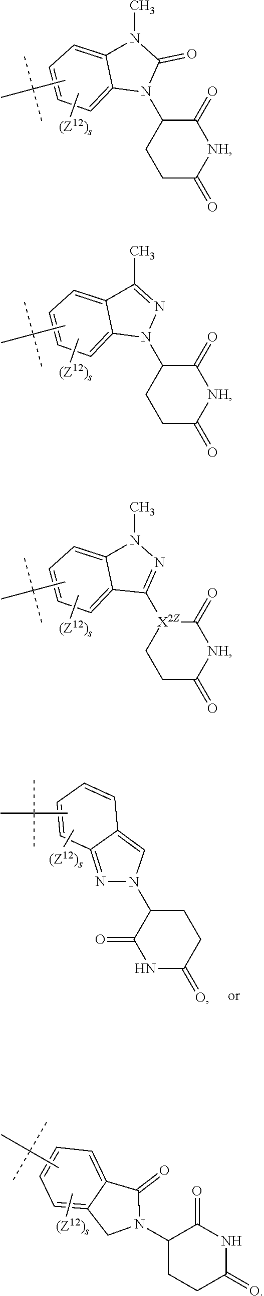

wherein Z¹² is halogen, C₁₋₃ alkoxy, or C₁₋₃ alkyl; wherein s is 0 or 1.

In an aspect, Z has the formula —S(═O)₂CHCH₂.

In an aspect, T optionally has the formula —C₄₋₆ heterocyloalkylene-C≡C—C₁₋₆ alkylene-C(═O)—; and Z has the Formula Z2:

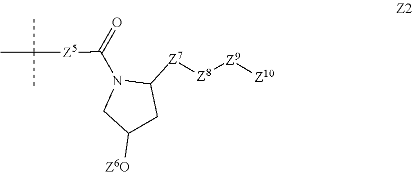

wherein
Z⁵ is —NZ¹¹—C₁₋₆ alkylene-, —O—C₁₋₆ alkylene-, —NZ¹¹—C₁₋₆ alkylene-, —NZ¹¹—C(═O)—C₁₋₆ alkylene-, —C(═O)—NZ¹¹—C₁₋₆ alkylene-, —NZ¹¹—C(═O)—NZ¹¹—C₁₋₆ alkylene-, —NZ¹¹—C(═O)—O—C₁₋₆ alkylene-, —S—C₁₋₆ alkylene-, or —NZ¹¹—S(═O)₂—C₁₋₆ alkylene-; wherein Z⁵ is optionally —C(═O)—NZ¹¹—C₁₋₆ alkylene-;
Z⁶ is H, —C(═O)—C₁₋₆ alkyl, —C(═O)—O—C₁₋₆ alkyl, —C(═O)—N(Z¹¹)₂, —P(═O)(Z¹¹)₂, or —P(═O)(OZ¹¹)₂; wherein Z⁶ is optionally H;
Z⁷ is —C(═O)—, —C(═O)—NZ¹¹—, —C(═O)—NZ¹¹—C₁₋₆ alkylene-, —S(═O)₂—, —S(═O)₂—NZ¹¹—;
Z⁸ is a covalent bond, —O—, —C(═O)—, —C(═S)—, —NZ¹¹—, —S(═O)—, or —S(═O)₂—;
Z⁹ is —C₅₋₆ arylene- or —C₃₋₅ heteroarylene-;
Z¹⁰ is —C₅₋₆ arylene- or —C₃₋₅ heteroarylene-; wherein Z¹⁰ is optionally substituted with —C₁₋₆ alkyl;
Z¹¹ is H or —C₁₋₆ alkyl.

In an aspect, the ubiquitin ligase binding moiety has the formula:

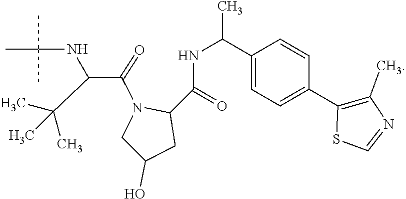

In an aspect, R² is H.

In an aspect, at least one of: X⁵ is CH; X⁷ is CH or N; X⁸ is CH; R^{4A} is H; each R⁵ independently is H or methyl; R⁶ is H; or R⁷ is H or —C₁₋₃ alkyl.

In an aspect, R^{1A} is F.

In an aspect, at least one hydrogen (H) is deuterium (D).

In an aspect, R¹ is

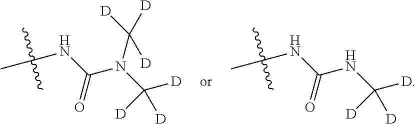

In an aspect, R^{1A} is deuterium; X⁵, X⁹, and X¹⁰ are each CR^{1B}; and the R^{1B} of each of X⁵, X⁹, and X¹⁰ is deuterium.

In an aspect, R³ is

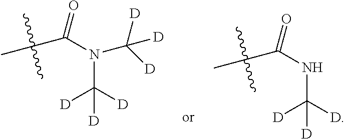

In an aspect, the Formula M is derived from a deuterated moiety of any of Examples D1, D2, D6, D31, D33, D128, or D132.

In an aspect, the Formula M is derived from any of Examples 1 to 758, any of Examples E1A to E434A, any of Examples V1 to V142, or any of Examples V1A to V187A.

In an aspect, the compound, or a pharmaceutically acceptable salt thereof, is one of Examples E1 to E732 or one of the prophetic examples of Table 5.

In an aspect, the compound, or a pharmaceutically acceptable salt is one of:
(S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide;
(S)—N-(4-(3,3-dimethylureido)-3-fluorobenzyl)-5-(4-(((1-((3-(2,4-dioxotetrahydropyrimidin-1(2H)-yl)-1-methyl-1H-indazol-7-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide;
rac-(R)-5-(4-(dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide;
(S)-5-(5-((2-(4-(3-(2,4-Dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-7-(4-((2-(4-(3-(2,4-dioxotetrahydropyrimidin-1(2H)-yl)-1-methyl-1H-indazol-6-yl)piperidin-1-yl)ethyl)(methyl)carbamoyl)phenyl)-5-methyl-N-(4-(methylcarbamoyl)benzyl)-3-(trifluoromethyl)-5,6,7,8-tetrahydroimidazo[1,5-a]pyrazine-1-carboxamide;
N-(4-(3,3-dimethylureido)benzyl)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-carbamoylbenzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-N-(4-(3-methyl-2-oxo-2,3-dihydro-1H-imidazol-1-yl)benzyl)-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(3-methylureido)benzyl)-4,5,6,7-tetrahydro-1H-pyrazolo[4,3-c]pyridine-3-carboxamide;
(S)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-isopropyl-7-methyl-N-(4-(pyridazin-3-ylamino)benzyl)-4,5,6,7-tetrahydro-1H-pyrazolo[4,3-c]pyridine-3-carboxamide;
rac-(R)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide;
N-(4-(3,3-dimethylureido)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-7,7-dimethyl-1-(2,2,2-trifluoroethyl)-4,5,6,7-tetrahydro-1H-pyrazolo[4,3-c]pyridine-3-carboxamide;
rac-(R)-5-(5-(1,3-dimethyl-5-oxo-1,5-dihydro-4H-1,2,4-triazol-4-yl)pyridin-2-yl)-N-(4-(3-(2-(4-(3-(2,6-dioxopiperidin-3-yl)-1-methyl-1H-indazol-6-yl)piperidin-1-yl)ethyl)-3-methylureido)-3-fluorobenzyl)-1-isopropyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
(S)—N-(4-(3,3-Dimethylureido)benzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-(((1-(3-(2,4-dioxotetrahydropyrimidin-1(2H)-yl)-1-methyl-1H-indazol-6-yl)piperidin-4-yl)methyl)(methyl)carbamoyl)pyridin-2-yl)-1-isopropyl-7-methyl-4,5,6,7-tetrahydro-1H-pyrazolo[4,3-c]pyridine-3-carboxamide;
(7S)-5-(5-{[2-(1-{[3-(2,4-dioxo-1,3-diazinan-1-yl)-1-methyl-1H-indazol-6-yl]methyl}piperidin-4-yl)ethyl](methyl)carbamoyl}pyridin-2-yl)-1-ethyl-N-{[4-(methylcarbamoyl)phenyl]methyl}-7-(trifluoromethyl)-4,5,6,7-tetrahydro-1H-pyrazolo[4,3-c]pyridine-3-carboxamide;
rac-N-(4-(3,3-dimethylureido)benzyl)-5-(5-((2-((3R,4S)-4-(3-(2,4-dioxotetrahydropyrimidin-1(2H)-yl)-1-methyl-1H-indazol-6-yl)-3-fluoropiperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(1-(2,6-dioxopiperidin-3-yl)-3-methyl-1H-indazol-5-yl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(4-((2,6-dioxopiperidin-3-yl)amino)-2-fluorophenyl)piperidin-1-yl)ethyl)(methyl)carbamoyl)pyridin-2-yl)-1-isopropyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-isopropyl-7,7-dimethyl-4,5,6,7-tetrahydro-1H-pyrazolo[4,3-c]pyridine-3-carboxamide;
(S)—N-(4-(3,3-dimethylureido)benzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-ethyl-7-methyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(4-(2,6-dioxopiperidin-3-yl)-2-fluorophenyl)piperazin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(1-(4-(2,6-dioxopiperidin-3-yl)-2-fluorophenyl)piperidin-4-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(4-(2,6-dioxopiperidin-3-yl)-2-fluorophenyl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide.

In an aspect, the compound, or a pharmaceutically acceptable salt is one of:
(S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide;
rac-(R)-5-(5-(Dimethylcarbamoyl)pyridin-2-yl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide;
rac-(R)-5-(4-(Dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide;
(S)—N-(4-(3,3-Dimethylureido)-3-fluorobenzyl)-5-(4-(((1-((3-(2,4-dioxotetrahydropyrimidin-1(2H)-yl)-1-methyl-1H-indazol-7-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-(((1-((3-(2,4-dioxotetrahydropyrimidin-1(2H)-yl)-1-methyl-1H-indazol-7-yl)methyl)piperidin-4-yl)methyl)(methyl)carbamoyl)pyridin-2-yl)-1-isopropyl-7-methyl-4,5,6,7-tetrahydro-1H-pyrazolo[4,3-c]pyridine-3-carboxamide;
(S)-5-(5-((2-(1-(3-(2,4-Dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)—N-(4-(3,3-dimethylureido)-3-fluorobenzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-N-(3-fluoro-4-(3-methylureido)benzyl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-carbamoylbenzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)-3-fluorobenzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-6-yl)piperazin-1-yl)ethyl)(methyl)carbamoyl)pyridin-2-yl)-1-isopropyl-7-methyl-N-(4-(3-methylureido)benzyl)-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(3-methylureido)benzyl)-4,5,6,7-tetrahydro-1H-pyrazolo[4,3-c]pyridine-3-carboxamide;
(S)—N-(4-carbamoylbenzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-5-fluoro-1-methyl-1H-indazol-6-yl)piperidin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-7-methyl-N-(4-(3-methyl-2-oxo-2,3-dihydro-1H-imidazol-1-yl)benzyl)-1-(2,2,2-trifluoroethyl)-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-7-methyl-1-(2,2,2-trifluoroethyl)-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-7-methyl-N-(4-(methylcarbamoyl)benzyl)-1-(2,2,2-trifluoroethyl)-4,5,6,7-tetrahydro-1H-pyrazolo[4,3-c]pyridine-3-carboxamide;
N-(4-(3,3-dimethylureido)benzyl)-5-(5-((2-(4-(3-(2,4-dioxotetrahydropyrimidin-1(2H)-yl)-1-methyl-1H-indazol-6-yl)piperazin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
N-(4-(3,3-dimethylureido)benzyl)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide;
N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7,7-dimethyl-4,5,6,7-tetrahydro-1H-pyrazolo[4,3-c]pyridine-3-carboxamide;
rac-(R)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide;
rac-(R)-5-(5-((2-(4-(3-(2,6-dioxopiperidin-3-yl)-1-methyl-1H-indazol-6-yl)piperazin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide;
N-(4-(3,3-dimethylureido)benzyl)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-isopropyl-7,7-dimethyl-4,5,6,7-tetrahydro-1H-pyrazolo[4,3-c]pyridine-3-carboxamide;
N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-7,7-dimethyl-1-(2,2,2-trifluoroethyl)-4,5,6,7-tetrahydro-1H-pyrazolo[4,3-c]pyridine-3-carboxamide;
5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-ethyl-7,7-dimethyl-N-(4-(methylcarbamoyl)benzyl)-4,5,6,7-tetrahydro-1H-pyrazolo[4,3-c]pyridine-3-carboxamide;
rac-(R)-5-(5-((2-(4-(3-(2,6-dioxopiperidin-3-yl)-1-methyl-1H-indazol-7-yl)piperazin-1-yl)ethyl)(methyl)carbamoyl)pyridin-2-yl)-1-ethyl-7,7-dimethyl-N-(4-(methylcarbamoyl)benzyl)-4,5,6,7-tetrahydro-1H-pyrazolo[4,3-c]pyridine-3-carboxamide;
5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-ethyl-7,7-dimethyl-N-(4-(methylcarbamoyl)benzyl)-4,5,6,7-tetrahydro-1H-pyrazolo[4,3-c]pyridine-3-carboxamide; or
N-(4-(3,3-dimethylureido)benzyl)-5-(4-((2-(4-(3-(2,4-dioxotetrahydropyrimidin-1(2H)-yl)-1-methyl-1H-indazol-6-yl)piperidin-1-yl)ethyl)(methyl)carbamoyl)phenyl)-1-ethyl-7,7-dimethyl-4,5,6,7-tetrahydro-1H-pyrazolo[4,3-c]pyridine-3-carboxamide.

In an aspect, the compound, or a pharmaceutically acceptable salt thereof, is one of:
(S)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
rac-(R)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide; or
rac-(R)-5-(5-((2-(4-(3-(2,6-dioxopiperidin-3-yl)-1-methyl-1H-indazol-6-yl)piperazin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide.

In an aspect, the compound, or a pharmaceutically acceptable salt is (S)—N-(4-(3,3-Dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, the compound is (S)—N-(4-(3,3-Dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, the compound is one of:
(7S)-5-(5-(dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide;
rac-(R)-5-(5-(dimethylcarbamoyl)pyridin-2-yl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide;
rac-(R)-5-(4-(dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide;
(7S)—N-(4-(3,3-dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide;
rac-(R)—N-(4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)-9-(3-(1-(2,6-dioxopiperidin-3-yl)-3-methyl-2-oxo-2,3-dihydro-1H-benzo[d]imidazol-5-yl)propyl)-3,9-diazaspiro[5.5]undecane-3-carboxamide;
(S)-5-(5-(dimethylcarbamoyl)pyridin-2-yl)-N-(4-(4-((1-(3-(1-((RS)-2,6-dioxopiperidin-3-yl)-3-methyl-2-oxo-2,3-dihydro-1H-benzo[d]imidazol-5-yl)propyl)piperidin-4-yl)oxy)piperidine-1-carboxamido)benzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide; or
(S)-5-(4-(dimethylcarbamoyl)phenyl)-1-isopropyl-7-methyl-N-(4-(4-(vinylsulfonyl)piperazine-1-carboxamido)benzyl)-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide. In an aspect, a compound or a pharmaceutically acceptable salt thereof; wherein the compound is (S)—N-(4-carbamoylbenzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is rac-(R)—N-(4-(3,3-dimethylureido)benzyl)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-Dimethylureido)benzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-dimethylureido)benzyl)-5-(5-(((1-(3-(2,4-dioxotetrahydropyrimidin-1(2H)-yl)-1-methyl-1H-indazol-6-yl)piperidin-4-yl)methyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is rac-N-(4-(3,3-dimethylureido)benzyl)-5-(5-((2-((3R,4S)-4-(3-(2,4-dioxotetrahydropyrimidin-1(2H)-yl)-1-methyl-1H-indazol-6-yl)-3-fluoropiperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is rac-(R)—N-(4-(3,3-dimethylureido)benzyl)-5-(5-((2-(4-(4-(2,6-dioxopiperidin-3-yl)-2-fluorophenyl)piperazin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is rac-(R)—N-(4-(3,3-dimethylureido)benzyl)-5-(5-((2-(4-(4-(2,6-dioxopiperidin-3-yl)-2-fluorophenyl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is rac-(R)-5-(4-(dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-((1-((3-(2,4-dioxotetrahydropyrimidin-1(2H)-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-dimethylureido)-3-fluorobenzyl)-5-(4-(((1-((3-(2,4-dioxotetrahydropyrimidin-1(2H)-yl)-1-methyl-1H-indazol-7-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-dimethylureido)benzyl)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-7-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-5-methyl-N-(4-(3-methylureido)benzyl)-3-(trifluoromethyl)-5,6,7,8-tetrahydroimidazo[1,5-a]pyrazine-1-carboxamide.

In an aspect, a compound or a pharmaceutically acceptable salt thereof; wherein the compound is (S)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-N-(4-(3-methylureido)benzyl)-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-7-(4-((2-(4-(3-(2,4-dioxotetrahydropyrimidin-1(2H)-yl)-1-methyl-1H-indazol-6-yl)piperidin-1-yl)ethyl)(methyl)carbamoyl)phenyl)-5-methyl-N-(4-(methylcarbamoyl)benzyl)-3-(trifluoromethyl)-5,6,7,8-tetrahydroimidazo[1,5-a]pyrazine-1-carboxamide.

In an aspect, a compound or a pharmaceutically acceptable salt thereof; wherein the compound is (S)-7-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-5-methyl-N-(4-(methylcarbamoyl)benzyl)-3-(trifluoromethyl)-5,6,7,8-tetrahydroimidazo[1,5-a]pyrazine-1-carboxamide.

In an aspect, a compound or a pharmaceutically acceptable salt thereof; wherein the compound is (S)—N-(4-(3,3-dimethylureido)-3-fluorobenzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-carbamoylbenzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-dimethylureido)benzyl)-5-(5-(((1-((3-(2,4-dioxotetrahydropyrimidin-1(2H)-yl)-1-methyl-1H-indazol-7-yl)methyl)piperidin-4-yl)methyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-N-(3-fluoro-4-(3-methylureido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide.

In an aspect, a compound or a pharmaceutically acceptable salt thereof; wherein the compound is 5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide.

In an aspect, a compound or a pharmaceutically acceptable salt thereof; wherein the compound is 5-(5-(dimethylcarbamoyl)pyridin-2-yl)-N-(4-(3-(2-(4-(3-(2,4-dioxotetrahydropyrimidin-1(2H)-yl)-1-methyl-1H-indazol-6-yl)piperidin-1-yl)ethyl)-3-methylureido)-3-fluorobenzyl)-1-isopropyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-(((1-((3-(2,4-dioxotetrahydropyrimidin-1(2H)-yl)-1-methyl-1H-indazol-7-yl)methyl)piperidin-4-yl)methyl)(methyl)carbamoyl)pyridin-2-yl)-1-isopropyl-7-methyl-N-(4-(3-methyl-2-oxo-2,3-dihydro-1H-imidazol-1-yl)benzyl)-4,5,6,7-tetrahydro-1H-pyrazolo[4,3-c]pyridine-3-carboxamide.

In an aspect, a compound or a pharmaceutically acceptable salt thereof; wherein the compound is rac-(R)-5-(5-(dimethylcarbamoyl)pyridin-2-yl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(3,3-dimethylureido)-3-fluorobenzyl)-5-(4-((2-(1-(1-((RS)-2,6-dioxopiperidin-3-yl)-3-methyl-2-oxo-2,3-dihydro-1H-benzo[d]imidazol-5-yl)piperidin-4-yl)ethyl)(methyl)carbamoyl)phenyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-((2-(4-(3-(2,4-dioxotetrahydropyrimidin-1(2H)-yl)-1-methyl-1H-indazol-7-yl)piperidin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide.

In an aspect, a compound or a pharmaceutically acceptable salt thereof; wherein the compound is (S)—N-(4-(1H-imidazol-2-yl)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-7-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-5-methyl-N-(4-(methylcarbamoyl)benzyl)-3-(trifluoromethyl)-5,6,7,8-tetrahydroimidazo[1,5-a]pyrazine-1-carboxamide.

In an aspect, a compound or a pharmaceutically acceptable salt thereof; wherein the compound is (S)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-isopropyl-7-methyl-N-(4-(3-methyl-2-oxo-2,3-dihydro-1H-imidazol-1-yl)benzyl)-4,5,6,7-tetrahydro-1H-pyrazolo[4,3-c]pyridine-3-carboxamide.

In an aspect, a compound or a pharmaceutically acceptable salt thereof; wherein the compound is (S)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-N-(4-(3-methyl-2-oxo-2,3-dihydro-1H-imidazol-1-yl)benzyl)-4,5,6,7-tetrahydro-1H-pyrazolo[4,3-c]pyridine-3-carboxamide.

In an aspect, a compound or a pharmaceutically acceptable salt thereof; wherein the compound is (S)-7-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-5-methyl-N-(4-(3-methyl-2-oxo-2,3-dihydro-1H-imidazol-1-yl)benzyl)-3-(trifluoromethyl)-5,6,7,8-tetrahydroimidazo[1,5-a]pyrazine-1-carboxamide.

In an aspect, a compound or a pharmaceutically acceptable salt thereof; wherein the compound is (7S)-5-(5-(dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is rac-(R)-5-(5-(dimethylcarbamoyl)pyridin-2-yl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is rac-(R)-5-(4-(dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is (7S)—N-(4-(3,3-dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is N-(4-(((S)-5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)-9-(3-(1-((RS)-2,6-dioxopiperidin-3-yl)-3-methyl-2-oxo-2,3-dihydro-1H-benzo[d]imidazol-5-yl)propyl)-3,9-diazaspiro[5.5]undecane-3-carboxamide.

In an aspect, a compound or a pharmaceutically acceptable salt thereof; wherein the compound is (S)-5-(5-(dimethylcarbamoyl)pyridin-2-yl)-N-(4-(4-((1-(3-(1-((RS)-2,6-dioxopiperidin-3-yl)-3-methyl-2-oxo-2,3-dihydro-1H-benzo[d]imidazol-5-yl)propyl)piperidin-4-yl)oxy)piperidine-1-carboxamido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (7S)-5-(5-(dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is rac-(R)-5-(5-(dimethylcarbamoyl)pyridin-2-yl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is rac-(R)-5-(4-(dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide.

In an aspect, a compound or a pharmaceutically acceptable salt thereof; wherein the compound is (7S)—N-(4-(3,3-dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is N-(4-(((S)-5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)-9-(3-(1-((RS)-2,6-dioxopiperidin-3-yl)-3-methyl-2-oxo-2,3-dihydro-1H-benzo[d]imidazol-5-yl)propyl)-3,9-diazaspiro[5.5]undecane-3-carboxamide.

In an aspect, a compound or a pharmaceutically acceptable salt thereof; wherein the compound is (S)-5-(5-(dimethylcarbamoyl)pyridin-2-yl)-N-(4-(4-((1-(3-(1-((RS)-2,6-dioxopiperidin-3-yl)-3-methyl-2-oxo-2,3-dihydro-1H-benzo[d]imidazol-5-yl)propyl)piperidin-4-yl)oxy)piperidine-1-carboxamido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide.

In an aspect, a compound or a pharmaceutically acceptable salt thereof; wherein the compound is (S)-5-(4-(dimethylcarbamoyl)phenyl)-1-isopropyl-7-methyl-N-(4-(4-(vinylsulfonyl)piperazine-1-carboxamido)benzyl)-4,5,6,7-tetrahydro-1H-pyrazolo[4,3-c]pyridine-3-carboxamide.

In an aspect, a compound or a pharmaceutically acceptable salt thereof is one of: (7S)-5-(5-(dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide; (7S)—N-(4-(3,3-dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide; N-(4-(((S)-5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)-9-(3-(1-((RS)-2,6-dioxopiperidin-3-yl)-3-methyl-2-oxo-2,3-dihydro-1H-benzo[d]imidazol-5-yl)propyl)-3,9-diazaspiro[5.5]undecane-3-carboxamide; (S)-5-(5-(dimethylcarbamoyl)pyridin-2-yl)-N-(4-(4-((1-(3-(1-((RS)-2,6-dioxopiperidin-3-yl)-3-methyl-2-oxo-2,3-dihydro-1H-benzo[d]imidazol-5-yl)propyl)piperidin-4-yl)oxy)piperidine-1-carboxamido)benzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide; or (S)-5-(4-(dimethylcarbamoyl)phenyl)-1-isopropyl-7-methyl-N-(4-(4-(vinylsulfonyl)piperazine-1-carboxamido)benzyl)-4,5,6,7-tetrahydro-1H-pyrazolo[4,3-c]pyridine-3-carboxamide.

In an aspect, the compound is one of: (7S)-5-(5-(dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide; (7S)—N-(4-(3,3-dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide; N-(4-(((S)-5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)-9-(3-(1-((RS)-2,6-dioxopiperidin-3-yl)-3-methyl-2-oxo-2,3-dihydro-1H-benzo[d]imidazol-5-yl)propyl)-3,9-diazaspiro[5.5]undecane-3-carboxamide; (S)-5-(5-(dimethylcarbamoyl)pyridin-2-yl)-N-(4-(4-((1-(3-(1-((RS)-2,6-dioxopiperidin-3-yl)-3-methyl-2-oxo-2,3-dihydro-1H-benzo[d]imidazol-5-yl)propyl)piperidin-4-yl)oxy)piperidine-1-carboxamido)benzyl)-1-isopropyl-7-methyl-4,5,6,7-tetrahydro-1H-pyrazolo[4,3-c]pyridine-3-carboxamide; (S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide; or (S)-5-(4-(dimethylcarbamoyl)phenyl)-1-isopropyl-7-methyl-N-(4-(4-(vinylsulfonyl)piperazine-1-carboxamido)benzyl)-4,5,6,7-tetrahydro-1H-pyrazolo[4,3-c]pyridine-3-carboxamide.

In an aspect, a pharmaceutical composition comprises the compound of Formula I or a pharmaceutically acceptable salt thereof, and at least one pharmaceutically acceptable excipient.

In an aspect, a method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprises administering to a subject in need thereof a therapeutically effective amount of the compound of Formula I, or a pharmaceutically acceptable salt thereof.

In an aspect, a compound of the disclosure, or a pharmaceutically acceptable salt thereof, for use as a medicament.

In an aspect, a compound of the disclosure, or a pharmaceutically acceptable salt thereof, can be for use in the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, a compound of the disclosure, or a pharmaceutically acceptable salt thereof, can be for use in the treatment of at least one of a dermatological condition or a respiratory condition.

In an aspect, use of a compound of the disclosure, or a pharmaceutically acceptable salt thereof, can be for the manufacture of a medicament for the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, use of a compound, or a pharmaceutically acceptable salt thereof, can be for use in the treatment of at least one of a dermatological condition or a respiratory condition.

In an aspect, a method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprises administering to a subject in need thereof a therapeutically effective amount of the compound of the any of the preceding aspects, or a pharmaceutically acceptable salt thereof. The method can be for treating atopic dermatitis. The method can be for treating asthma.

In an aspect, a compound according to any of the preceding aspects, or a pharmaceutically acceptable salt thereof, is for use as a medicament.

In an aspect, a compound according to any of the preceding aspects, or a pharmaceutically acceptable salt thereof, is for use in the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, a compound according to any of the preceding aspects, or a pharmaceutically acceptable salt thereof, is for use in the treatment of at least one of a dermatological condition or a respiratory condition.

In an aspect, a use of a compound according to any of the preceding aspects, or a pharmaceutically acceptable salt thereof, for the manufacture of a medicament for the treatment of atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease (COPD), chronic pruritis, chronic urticaria, Crohn's disease (CD), dermatitis, diabetic kidney disease, eosinophilic esophagitis, fungal keratitis, gout, idiopathic pulmonary fibrosis (IPF), a joint disorder, keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa.

In an aspect, a use of a compound according to any of the preceding aspects, or a pharmaceutically acceptable salt thereof, is for use in the treatment of at least one of a dermatological condition or a respiratory condition.

Each of the aspects described herein may be combined with any other aspect(s) described herein not inconsistent with the aspect(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the aspect(s) described herein.

Furthermore, each of the aspects described herein envisions within its scope pharmaceutically acceptable salts of the compounds described herein.

Unless otherwise defined herein, scientific and technical terms used in connection with the present disclosure have the meanings that are commonly understood by those of ordinary skill in the art.

The disclosure described herein suitably may be practiced in the absence of any element(s) not specifically disclosed herein.

The compound(s) of the disclosure refers to the compounds of Formula I, Formula IA, Formula IB, Formula IC, Formula ID, and Formula IE, where a compound of Formula I can be a compound specifically of Formula IA, Formula IB, Formula IC, Formula ID, or Formula IE. One of ordinary skill in the art will appreciate that the compounds of the disclosure include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that the compounds of the disclosure include solvates, hydrates, isomorphs, polymorphs, esters, salt forms, prodrugs, and isotopically labelled versions thereof, where they may be formed. Compounds of the disclosure can include novel intermediates used in the preparation thereof.

As used herein, the singular form “a”, “an”, and “the” include plural references unless indicated otherwise. For example, “a” substituent includes one or more substituents. The term “or” means “and/or” unless clearly indicated otherwise.

As used herein, the term “about” when used to modify a numerically defined parameter means that the parameter may vary by as much as 10% below or above the stated numerical value for that parameter. For example, a dose of about 5 mg means 5%±10%, i.e., it may vary between 4.5 mg and 5.5 mg.

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo, and iodo (F, Cl, Br, I). More specifically, halogen can refer to fluoro and chloro.

“Cyano” refers to a substituent having a carbon atom joined to a nitrogen atom by a triple bond, i.e., —C≡N. “Cyanoimine” refers to a substituent having a carbon atom joined to a nitrogen atom by a double bond and that is bound to a cyano group, i.e., —C(R)═N—C≡N, where R can be a dimethylamino group, i.e., —C(N(CH₃)₂)═N—C≡N.

“Hydroxy” refers to an —OH group.

“Oxo” refers to a double bonded oxygen (═O).

“Thioxo” refers to a double bonded sulfur (═S).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁₋₁₂ alkyl”), 1 to 8 carbon atoms (“C₁₋₈ alkyl”), 1 to 6 carbon atoms (“C₁₋₆ alkyl”), 1 to 5 carbon atoms (“C₁₋₅ alkyl”), 1 to 4 carbon atoms (“C₁₋₄ alkyl”), 1 to 3 carbon atoms (“C₁₋₃ alkyl”), or 1 to 2 carbon atoms (“C₁₋₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Haloalkyl” refers to an alkyl group as defined above containing the specified number of carbon atoms wherein at least one hydrogen atom has been replaced by halogen. Haloalkyl groups man contain, but are not limited to, 1-6 carbon atoms (“C₁₋₆ haloalkyl”), 1-4 carbon atoms (“C₁₋₄ haloalkyl”), or 1-2 carbon atoms (“C₁₋₂ haloalkyl”). More specifically, fluorinated alkyl groups may be specifically referred to as “fluoroalkyl.” Examples of fluoroalkyl groups include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Hydroxyalkyl” refers to an alkyl group as defined above containing the specified number of carbon atoms wherein at least one hydrogen atom has been replaced by hydroxy (OH). Hydroxyalkyl groups man contain, but are not limited to, 1-6 carbon atoms (“C₁₋₆ hydroxyalkyl”), 1-4 carbon atoms (“C₁₋₄ hydroxyalkyl”), or 1-2 carbon atoms (“C₁₋₂ hydroxyalkyl”).

“Oxoalkyl” refers to an alkyl group as defined above containing the specified number of carbon atoms wherein at least two hydrogen atoms have been replaced by an oxo group. Oxoalkyl groups man contain, but are not limited to, 1-6 carbon atoms (“C₁₋₆ oxoalkyl”), 1-4 carbon atoms (“C₁₋₄ oxoalkyl”), or 1-2 carbon atoms (“C₁₋₂ oxoalkyl”).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁₋₈ alkoxy”), 1 to 6 carbon atoms (“C₁₋₆ alkoxy”), 1 to 4 carbon atoms (“C₁₋₄ alkoxy”), or 1 to 3 carbon atoms (“C₁₋₃ alkoxy”). Alkoxy groups include, but are not limited to, methoxy, ethoxy, n-propoxy, isobutoxy, and the like.

When “ene” is added after “yl” at the end a term to form a new term, the new term refers to a diradical formed by removing one hydrogen atom from the original term of which the new term derived. For example, an alkylene refers to a diradical group formed by removing one hydrogen atom from an alkyl group and that a “methylene” refers to a divalent radical —CH₂— derived from removing one hydrogen atom from methyl. More examples of such diradicals include, but are not limited to: alkenylene, alkynylene, cycloalkylene, phenylene, heterocyclylene, and heteroarylene which are derived from alkenyl, alkynyl, cycloalkyl, phenyl, heterocyclyl, and heteroaryl. “Alkylene” refers to a saturated, divalent aliphatic hydrocarbon radical that has a specified number of carbon atoms, including straight chain or branched chain groups. Alkylene groups may contain, but are not limited to, 1 to 12 carbon atoms (“C₁₋₁₂ alkylene”), 1 to 8 carbon atoms (“C₁₋₁₂ alkylene”), 1 to 6 carbon atoms (“C₁₋₆ alkylene”), 1 to 5 carbon atoms (“C₁₋₅ alkylene”), 1 to 4 carbon atoms (“C₁₋₄ alkylene”), 1 to 3 carbon atoms (“C₁₋₃ alkylene”), or 1 to 2 carbon atoms (“C₁₋₂ alkylene”). Non-limiting examples of “C₁₋₃ alkylene” include: —CH₂—, —CH(CH₃)—, —CH₂—CH₂—, —CH₂—CH₂—CH₂—, —CH(CH₃)—CH₂—, and —CH(CH₂CH₃)—. For a cyclic moiety, the removal of the hydrogen can occur on any atom of sufficient valency.

“Cycloalkyl” refers to a fully or partially saturated hydrocarbon ring system that has the specified number of carbon atoms, which may be a monocyclic, bridged or fused bicyclic, or polycyclic ring system that is connected to the base molecule through a carbon atom of the cycloalkyl ring. “Cycloalkyl” can refer to the ring system being a fully saturated hydrocarbon ring system. Cycloalkyl groups may contain, but are not limited to, 3 to 12 carbon atoms (“C₃₋₁₂ cycloalkyl”), 3 to 8 carbon atoms (“C₃₋₈ cycloalkyl”), 3 to 6 carbon atoms (“C₃₋₆ cycloalkyl”), 3 to 5 carbon atoms (“C₃₋₅ cycloalkyl”) or 3 to 4 carbon atoms (“C₃₋₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

“Heterocycloalkyl” refers to a fully or partially saturated ring system containing the specified number of ring atoms and containing at least one heteroatom selected from N, O, and S as a ring member, where ring S atoms are optionally substituted by one or two oxo groups (i.e., S(═O)_{q}, where q is 0, 1 or 2) and where the heterocycloalkyl ring is connected to the base molecule via a ring atom, which may be C or N. The heterocycloalkyl can refer to the ring system being fully saturated. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated, or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(═O)_{q} as ring members, or 1 to 2 ring heteroatoms, provided that such heterocycloalkyl rings do not contain two contiguous oxygen or sulfur atoms.

Heterocycloalkyl rings may be optionally substituted or unsubstituted, as further defined herein. Such substituents may be present on the heterocyclic ring attached to the base molecule, or on a spirocyclic, bridged or fused ring attached thereto. Heterocycloalkyl rings may include, but are not limited to, 3-8 membered heterocyclyl groups, for example 4-7 or 4-6 membered heterocycloalkyl groups, in accordance with the definition herein.

“Aryl” or “aromatic” refers to monocyclic, bicyclic (e.g., biaryl, fused), or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆₋₂₀ aryl”), 6 to 14 carbon atoms (“C₆₋₁₄ aryl”), 6 to 12 carbon atoms (“C₆₋₁₂ aryl”), or 6 to 10 carbon atoms (“C₆₋₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused), or polycyclic ring systems that contain the specified number of ring atoms and include at least one heteroatom selected from N, O, and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

“Amino” refers to a group —NH₂, which is unsubstituted or substituted. When substituted, the term includes groups of the form —NRₓR_{y}, one of Rₓ and R_{y} is an alkyl moiety and the other is H, or both of Rₓ and R_{y} are alkyl moieties, where the alkyl moieties have the specified number of carbon atoms (e.g., —NH(C₁₋₃ alkyl) or —N(C₁₋₃ alkyl)₂). The term “aminoheterocycloalkyl” refers to a heterocycloalkyl substituted with an amino group.

The term “pharmaceutically acceptable” means the substance (e.g., the compounds described herein) and any salt thereof, or composition containing the substance or salt of the compounds of the disclosure is suitable for administration to a subject or patient.

A “pharmaceutical composition” refers to a mixture of one or more of the compounds of the disclosure, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically can have a deuterium enrichment factor of, at least 1,000 (15% deuterium incorporation), at least 2,000 (30% deuterium incorporation), at least 3,000 (45% deuterium incorporation), at least 3,500 (52.5% deuterium incorporation), at least 3,500 (52.5% deuterium incorporation at each designated deuterium atom), at least 4,000 (60% deuterium incorporation), at least 4,500 (67.5% deuterium incorporation), at least 5,000 (75% deuterium incorporation), at least 5,500 (82.5% deuterium incorporation), at least 6,000 (90% deuterium incorporation), at least 6,333.3 (95% deuterium incorporation), at least 6,466.7 (97% deuterium incorporation), at least 6,600 (99% deuterium incorporation), or at least 6,633.3 (99.5% deuterium incorporation).

“Excipient” as used herein describes any ingredient other than the compound(s) of the disclosure. The choice of excipient will to a large extent depend on factors such as the mode of administration, the effect of the excipient on solubility and stability, and the nature of the dosage form. As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof. Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat”, or “treatment” as used herein embraces both preventative, i.e., prophylactic, and palliative treatment, i.e., relieve, alleviate, or slow the progression of the patient's disease (or condition) or any tissue damage associated with the disease.

As used herein, the term, “subject”, “individual”, or “patient,” used interchangeably, refers to any animal, including mammals. Mammals according to the disclosure include canine, feline, bovine, caprine, equine, ovine, porcine, rodents, lagomorphs, primates, humans, and the like, and encompass mammals in utero. Humans can be suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition, or disorder in an individual that may be predisposed to the disease, condition, or disorder but does not yet experience or display the pathology or symptomatology of the disease;
(2) inhibiting the disease; for example, inhibiting a disease, condition, or disorder in an individual that is experiencing or displaying the pathology or symptomatology of the disease, condition, or disorder (i.e., arresting (or slowing) further development of the pathology or symptomatology or both); and
(3) ameliorating the disease; for example, ameliorating a disease, condition, or disorder in an individual that is experiencing or displaying the pathology or symptomatology of the disease, condition, or disorder (i.e., reversing the pathology or symptomatology or both).

Salts encompassed within the term “pharmaceutically acceptable salts” refer to the compounds of this disclosure, which are generally prepared by reacting the free base or free acid with a suitable organic or inorganic acid, or a suitable organic or inorganic base, respectively, to provide a salt of the compound of the disclosure that is suitable for administration to a subject or patient.

In addition, the compound of the disclosure may also include other salts of such compounds which are not necessarily pharmaceutically acceptable salts, which may be useful as intermediates for one or more of the following: 1) preparing a compound of the disclosure; 2) purifying the compound of the disclosure; 3) separating enantiomers of the compound of the disclosure; or 4) separating diastereomers of the compound of the disclosure.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts. The salt can be a trifluoroacetate salt or a formate salt.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of the compounds of the disclosure may be prepared by methods well known to one skilled in the art, including but not limited to the following procedures
(i) by reacting a compound of the disclosure with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of a compound of the disclosure to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

The compounds of the disclosure, and pharmaceutically acceptable salts thereof, may exist in unsolvated and solvated forms. The term ‘solvate’ is used herein to describe a molecular complex comprising the compound of the disclosure, or a pharmaceutically acceptable salt thereof, and one or more pharmaceutically acceptable solvent molecules, for example, ethanol. The term ‘hydrate’ is employed when said solvent is water.

In addition, the compounds of the disclosure may also include other solvates of such compounds which are not necessarily pharmaceutically acceptable solvates, which may be useful as intermediates for one or more of the following: 1) preparing the compounds of the disclosure; 2) purifying the compounds of the disclosure; 3) separating enantiomers of the compounds of the disclosure; or 4) separating diastereomers of the compounds of the disclosure.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the disclosure are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

The compounds of the disclosure may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N—N⁺(CH₃)₃) polar head group. For more information, see Crystals and the Polarizing Microscope by N. H. Hartshorne and A. Stuart, 4^{th} Edition (Edward Arnold, 1970).

Compounds of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the disclosure containing one or more asymmetric carbon atoms may exist as two or more stereoisomers. Where a compound of the disclosure contains an alkenyl or alkenylene group, geometric cis/trans (or Z/E) isomers are possible. Cis/trans isomers may also exist for saturated rings.

The pharmaceutically acceptable salts of the compounds of the disclosure may also contain a counterion which is optically active (e.g., d-lactate or I-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the disclosure (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

Where structural isomers are interconvertible via a low energy barrier, tautomeric isomerism (‘tautomerism’) may occur. This may take the form of proton tautomerism in the compounds of the disclosure containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the disclosure have been drawn herein in a single tautomeric form, all possible tautomeric forms are included within the scope of the disclosure.

The present disclosure includes all pharmaceutically acceptable isotopically-labeled compounds of Formula I wherein one or more atoms are replaced by atoms having the same atomic number, but an atomic mass or mass number different from the atomic mass or mass number which predominates in nature.

Examples of isotopes suitable for inclusion in the compounds of Formula I may include isotopes of hydrogen, such as ²H (D, deuterium) and ³H (T, tritium), carbon, such as ¹¹C, ¹³C and ¹⁴C, chlorine, such as ³⁶C₁, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S

Certain isotopically-labelled compounds of Formula I, for example, those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C ¹⁸F, ¹⁵O and ¹³N, may be useful in Positron Emission Topography (PET) studies for examining substrate receptor occupancy. Substitution with deuterium, i.e., ²H, may afford certain therapeutic advantages resulting from greater metabolic stability, for example, increased in vivo half-life, reduced dosage requirements, reduced CYP450 inhibition (competitive or time dependent), or an improvement in therapeutic index or tolerability.

The disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of Formula I may be defined by the deuterium enrichment factor. It is understood that one or more deuterium may exchange with hydrogen under physiological conditions.

The deuterium compound can be selected from any one of the compounds of the examples specifically illustrated in the Examples section.

One or more hydrogen atoms on certain metabolic sites on the compounds of Formula I can be deuterated.

Isotopically-labeled compounds of Formula I may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

A compound of the disclosure may be administered in the form of a prodrug. Thus, certain derivatives of a compound of the disclosure which may have little or no pharmacological activity themselves may, when administered into or onto the body, be converted into a compound of the disclosure having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the disclosure may, for example, be produced by replacing appropriate functionalities present in the compounds of the disclosure with certain moieties known to those skilled in the art as ‘pro-moieties’ as described, for example, in ‘Design of Prodrugs’ by H. Bundgaard (Elsevier, 1985).

Thus, a prodrug in accordance with the disclosure may be (a) an ester or amide derivative of a carboxylic acid when present in a compound of the disclosure; (b) an ester, carbonate, carbamate, phosphate or ether derivative of a hydroxyl group when present in a compound of the disclosure; (c) an amide, imine, carbamate or amine derivative of an amino group when present in a compound of the disclosure; (d) a thioester, thiocarbonate, thiocarbamate or sulfide derivatives of a thiol group when present in a compound of the disclosure; or (e) an oxime or imine derivative of a carbonyl group when present in a compound of the disclosure.

Some specific examples of prodrugs in accordance with the disclosure include:
(i) when a compound of the disclosure contains a carboxylic acid functionality (—COOH), an ester thereof, such as a compound wherein the hydrogen of the carboxylic acid functionality of the compound is replaced by C₁₋₈ alkyl (e.g., ethyl) or (—C₁₋₃ alkyl)C(═O)OCH₂— (e.g., ^{t}BuC(═O)OCH₂—);
(ii) when a compound of the disclosure contains an alcohol functionality (—OH), an ester thereof, such as a compound wherein the hydrogen of the alcohol functionality of the compound is replaced by —CO(C₁₋₈ alkyl) (e.g., methylcarbonyl) or the alcohol is esterified with an amino acid;
(iii) when a compound of the disclosure contains an alcohol functionality (—OH), an ether thereof, such as a compound wherein the hydrogen of the alcohol functionality of the compound is replaced by (C₁₋₈ alkyl)C(═O)OCH₂— or —CH₂OP(═O)(OH)₂;
(iv) when a compound of the disclosure contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the disclosure contains a primary or secondary amino functionality (—NH₂ or —NHR where R #H), an amide thereof, for example, a compound wherein, as the case may be, one or both hydrogens of the amino functionality of the compound is/are replaced by (C₁₋₁₀)alkanoyl, —COCH₂NH₂ or the amino group is derivatized with an amino acid;
(vi) when a compound of the disclosure contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the disclosure may themselves act as prodrugs of other compounds the disclosure it is also possible for two compounds of the disclosure to be joined together in the form of a prodrug. In certain circumstances, a prodrug of a compound of the disclosure may be created by internally linking two functional groups in a compound of the disclosure, for instance by forming a lactone.

Also included within the scope of the disclosure are active metabolites of compounds of the disclosure, that is, compounds formed in vivo upon administration of the drug, often by oxidation or dealkylation. Some examples of metabolites in accordance with the disclosure include, but are not limited to,
(i) where the compound of the disclosure contains an alkyl group, a hydroxyalkyl derivative thereof (—CH á —COH):
(ii) where the compound of the disclosure contains an alkoxy group, a hydroxy derivative thereof (—OR á —OH);
(iii) where the compound of the disclosure contains a tertiary amino group, a secondary amino derivative thereof (—NRR′ á —NHR or —NHR′);
(iv) where the compound of the disclosure contains a secondary amino group, a primary derivative thereof (—NHR á —NH₂);
(v) where the compound of the disclosure contains a phenyl moiety, a phenol derivative thereof (-Ph á -PhOH);
(vi) where the compound of the disclosure contains an amide group, a carboxylic acid derivative thereof (—CONH₂ á COOH); and
(vii) where the compound contains a hydroxy or carboxylic acid group, the compound may be metabolized by conjugation, for example, with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

The compounds of the disclosure are targeted protein ligands that are covalently linked to E3 ligase ligands or ligands known to interact with the ubiquitin proteasome system (Degrons), through a Linker of varying length and functionality. The compounds of the disclosure that include the Degrons can be bifunctional compounds, targeting two different proteins.

The compounds of the disclosure have the general structure: Ubiquitin Ligase Binding Moiety-Linker-Targeting Moiety, wherein the Linker is covalently bound to at least one Ubiquitin Ligase Binding Moiety and at least one Targeting Moiety (M). The Ubiquitin Ligase Binding Moiety (Z), also referred to herein more broadly as a Degron, can bind to a ubiquitin ligase such as an E3 Ubiquitin Ligase (e.g., cereblon (CRBN), von Hippel-Lindau (VHL), and the like). The Targeting Moiety (M) can bind to the targeted protein, STAT6. Such compounds of the present disclosure are generally presented as compounds of Formula I.

M-T-Z I

The Degron is small in size and highly effective in recruiting targeted proteins for degradation. The Degron is a compound that serves to link a targeted protein, through the Linker and Targeting Moiety, to a ubiquitin ligase for proteasomal degradation. The Degron can be a compound that is capable of binding to or binds to a ubiquitin ligase. The Degron can be a compound that is capable of binding to or binds to a E3 Ubiquitin Ligase, including cereblon, wherein the Degron is a thalidomide, lenalidomide, pomalidomide, or iberdomide, or newer IMiDs CRBN ligands disclosed in WO2019/060693, WO2019/140387, WO2019/236483 or analogs thereof. The Degron can be a compound that is capable of binding to or binds to a E3 Ubiquitin Ligase, including von Hippel-Lindau ligand. See, e.g., WO2020/092907; WO2013106643; Buckley et al. J. Am. Chem. Soc. 2012, 134, 4465-4468, “Targeting the Von Hippel-Lindau E3 Ubiquitin Ligase Using Small Molecules to Disrupt the VHL/Hif-1alpha Interaction”, Soares et al. J. Med. Chem. 2019, 61, 599-618, “Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298)”. The Degron can be a compound that is capable of binding to or binds to a E3 Ubiquitin Ligase, including inhibitors of apoptosis protein ligases (IAP1, IAP2, XIAP). See, e.g., Itoh et al, J. Am. Chem. Soc. 2010, 132, 5820-5826 “Protein Knockdown Using Methyl Bestatin-Ligand Hybrid Molecules: Design and Synthesis of Inducers of Ubiquitination-Mediated Degradation of Cellular Retinoic Acid-Binding Proteins', Mares et al. Commun. Biol. 2020, 3, 140, “Extended pharmacodynamic responses observed upon PROTAC-mediated degradation of RIPK2′, and Tinworth et al. ACS Chem. Biol. 2019, 14, 342-347, “PROTAC-Mediated Degradation of Bruton's Tyrosine Kinase Is Inhibited by Covalent Binding.’ The Degron can be a compound that is capable of binding to or binds other ubiquitin proteasome proteins that can induce degradation including, but not limited to, the Hsp70/90 chaperone complex (WO2020/207395), Usp14 (WO2019/238886), UchL5 (WO2019238816), and Rpn11 (WO2019/238817). The ubiquitin ligase binding moiety can be an E3 ubiquitin ligase binding moiety, for example, a cereblon E3 ubiquitin ligase binding moiety, a VHL E3 ubiquitin ligase binding moiety, an IAP E3 ubiquitin ligase binding moiety, or an MDM2 E3 ubiquitin ligase binding moiety. The ubiquitin ligase binding moiety can be a ubiquitin ligase binding moiety as disclosed in WO2024/064080.

The ubiquitin ligase binding moiety Z can be —S(═O)₂CHCH₂ or can have the Formula Z1.

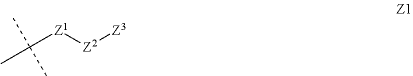

Z¹ can be —C₆₋₁₀ arylene-, —C₆₋₁₀ heteroarylene-, —C₃₋₁₄ heterocycloalkylene-, or two —C₃₋₁₄ heterocycloalkylene- rings joined together by a covalent bond. Z¹ can be —C₃₋₁₄ heterocycloalkylene- or —C₆₋₁₀ arylene- (for example, -phenylene-). Z¹ can be —C₆₋₈ heteroarylene- or —C₅₋₆ arylene-, wherein the heteroarylene or heterocycloalkylene groups of Z¹ can each independently include 1, 2, or 3 heteroatoms of N. Z¹ is optionally substituted with one or two or three of halogen, —C₁₋₆ fluoroalkyl, —C₁₋₆ alkyl, C₁₋₃ alkoxy, or oxo. The —C₃₋₁₄ heterocycloalkylene- can be optionally substituted with one, two, or three of a C₁₋₆ alkyl, cyano, halogen, hydroxy, ═NH, ═N—C₁₋₃ alkyl, C₁₋₃ methoxy, oxo, thioxo, —NO₂, —N(R^{1Z})₂, —NR^{1Z}C(═O)R^{1Z}, —NR^{1Z}C(═O)NR^{1Z})₂, —NR^{1Z}C(═O)OR^{1Z}, —NR^{1Z}S(═O)₂R^{1Z}, —C(═O)R^{1Z}, —C(═O)OR^{1Z}, —C(═O)(NR^{1Z})₂, —C(R^{1Z})₂N(R^{1Z})C(═O)R^{1Z}, —C(R^{1Z})₂N(R^{1Z})C(═O)NR^{1Z}, —C(═O)N(R^{1Z})OR^{1Z}, —OC(═O)R^{1Z}, —OC(═O)N(R^{1Z})₂, —OP(═O)(R^{1Z})₂—OP(═O)(OR^{1Z})₂, —OP(═O)(OR^{1Z})(N(R^{1Z})₂), —OP(═O)(N(R^{1Z})₂)₂, —SR^{1Z}, —S(═O)R^{1Z}, —S(═O)₂R^{1Z}, —S(═O)₂NR^{1Z}, —S(═O)R^{1Z}, —Si(R^{1Z})₃, —NP(═O)(R^{1Z})₂, —N(R^{1Z})P(═O)(OR^{1Z})₂, —N(R^{1Z})P(═O)(OR^{1Z})(NR^{1Z}R^{1Z}), —N(R^{1Z})P(═O)(NR^{1Z}R^{1Z})₂, or —N(R^{1Z})S(═O)₂R^{1Z}. The two heterocyclic rings joined together by a covalent bond include a first heterocyclic ring and a second heterocyclic ring. The first heterocyclic ring is a bivalent heterocyclic ring and second heterocyclic ring is either bivalent and located in between the first heterocyclic ring and Z² or the second heterocyclic ring is a substituent of the first heterocyclic ring. The first heterocyclic ring is a bivalent C₃₋₇ heterocyclyl that is optionally substituted with one, two, or three of R^{1Z}, ═NR^{1Z}, oxo, or thioxo. The second heterocyclic ring can be a bivalent C₃₋₇ heterocyclyl or a monovalent C₃₋₇ heterocyclyl, where the second heterocyclic ring is optionally substituted with one, two, or three of R^{1Z}, ═NR^{1Z}, oxo, or thioxo. The first heterocyclic ring and second heterocyclic rings can each independently include one, two, or three heteroatoms of N, S, or O.
Z¹ can be —C₆₋₈ heteroarylene- or —C₅₋₆ arylene-. Z¹ can optionally be substituted with one or two or three of halogen, —C₁₋₃ fluoroalkyl, —C₁₋₃ alkyl, C₁₋₃ alkoxy, or oxo. The heteroarylene or heterocycloalkylene groups of Z¹ can each independently include 1, 2, or 3 heteroatoms of N.
Z² can be the covalent bond, —NH—, or —CH₂—.
X^{1Z} can be C(═O), X^{2Z} can be CH or N, and X^{3Z} can be CH₂;

Each R^{1Z} independently can be H, —C₁₋₆ alkyl, phenyl, or —C₃₋₅ heterocyclyl. The —C₃₋₅ heterocyclyl can have 1 to 4 heteroatoms selected from N, O, or S. Two neighboring R^{1Z} can be taken together to form a C₃₋₆ heterocyclyl having at least one N and optionally 1 to 2 additional heteroatoms selected from N, O, or S.
Z² can be a covalent bond, —NH—, —O—, —NCH₃—, —C(═O)—NH—, —CH₂—, or —C₂₋₃ alkylene-, wherein 1 of the methylene units of the —C₁₋₃ alkylene- is optionally replaced with —O—, —C(═O)—, —C(═S)—, —C(R^{1Z})₂—, —CH(R^{1Z})—, —C(F)₂—, —N(R^{1Z})—, —S—, —S(O)₂— or —(C)═CH—. Z² can be a covalent bond. Z² can be —NH—. Z² can be —CH₂—.
Z³ can have the Formula Z^{3-a}

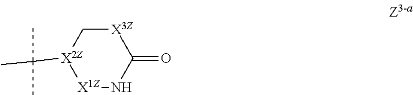

X^{1Z} can be a covalent bond, CH₂, C(═O), CHCF₃, SO₂, S(═O), P(═O)C₁₋₃ alkyl, P(═O)OC₁₋₃alkyl, P(═O)N(C₁₋₃ alkyl)₂, C(═S), or C₃₋₄ heteroaryl. X^{1Z} can be C(═O).
X^{2Z} can be CH, N, or SiH. X^{2Z} can be CH or N.
X^{3Z} can be CH₂, NH, O, S, or Si(C₁₋₃ alkyl)₂. X^{3Z} can be CH₂.
X^{1Z} can be C(═O), X^{2Z} can be CH or N, and X^{3Z} can be CH₂.
Z³ can have the Formula Z^{3-b}

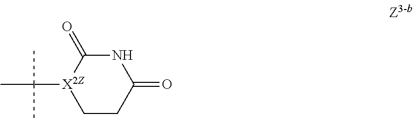

The Ubiquitin Ligase Binding Moiety can have the Formula ZI; wherein Z¹ can be —C₆₋₈ heteroarylene- or —C₅₋₆ arylene-; wherein Z¹ can optionally be substituted with one or two or three of halogen, —C₁₋₃ fluoroalkyl, —C₁₋₃ alkyl, C₁₋₃ alkoxy, or oxo; wherein the heteroarylene or heterocycloalkylene groups of Z¹ can each independently include 1, 2, or 3 heteroatoms of N; Z² can be the covalent bond, —NH—, or —CH₂—; Z³ can have the Formula Z^{3-b}; and X^{2Z} can be CH or N.

The Ubiquitin Ligase Binding Moiety can be attached to the Linker via a carbon or nitrogen atom that is a ring member of Z¹ or that is a substituent atom to the ring of Z¹.

The Ubiquitin Ligase Binding Moiety can be one of the following:

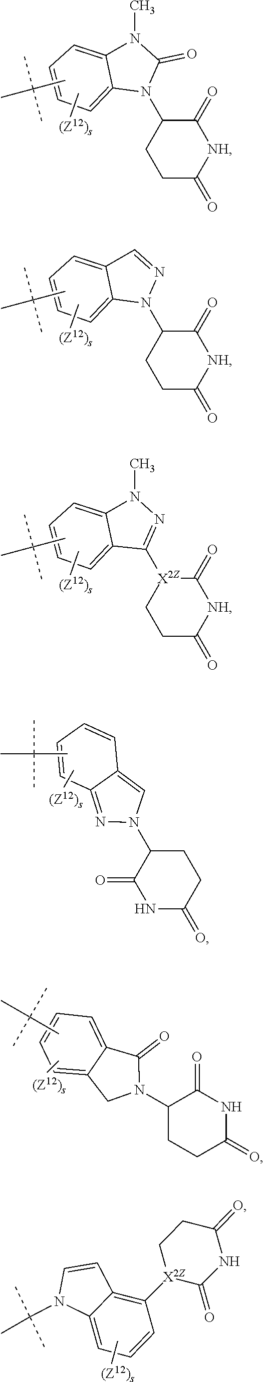

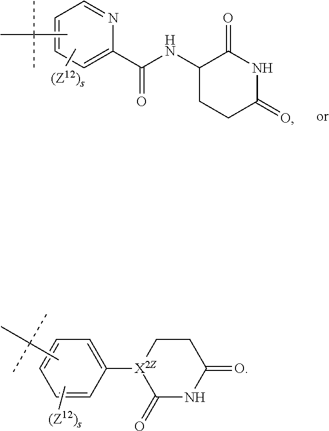

wherein Z¹² is halogen, C₁₋₃ alkoxy, or C₁₋₃ alkyl; and wherein s is 0 or 1.

The Ubiquitin Ligase Binding Moiety can be one of the following:

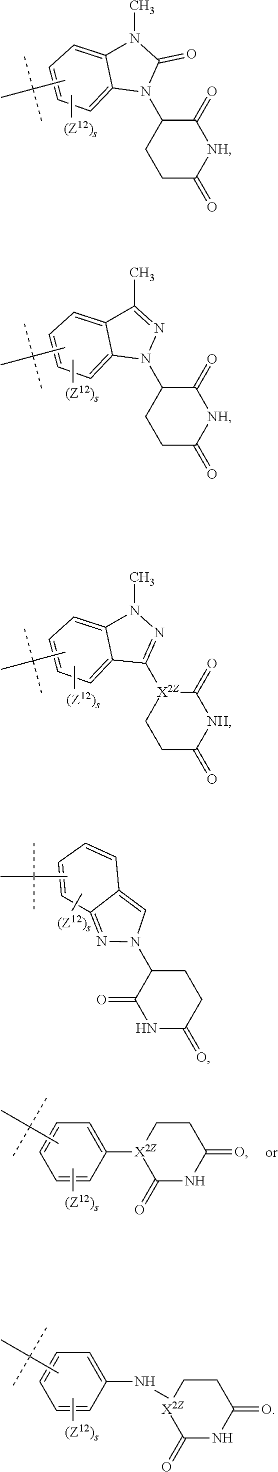

wherein Z¹² is halogen, C₁₋₃ alkoxy, or C₁₋₃ alkyl; and s is 0 or 1.

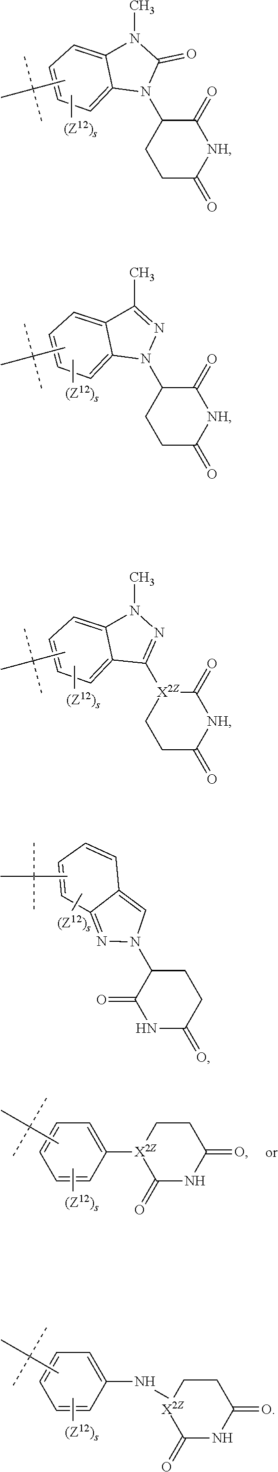

wherein Z¹² is halogen, C₁₋₃ alkoxy, or C₁₋₃ alkyl; and s is 0 or 1.

The ubiquitin ligase binding moiety can have the Formula Z². The ubiquitin ligase binding moiety can have the Formula Z² and T can have the formula —C₄₋₆ heterocyloalkylene-C≡C—C₁₋₆ alkylene-C(═O)—.

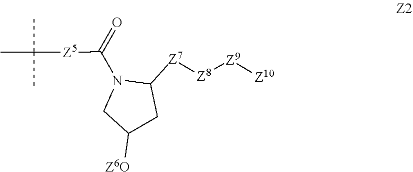

Z⁵ can be —NZ¹¹—C₁₋₆ alkylene-, —O—C₁₋₆ alkylene-, —NZ¹¹—C₁₋₆ alkylene-, —NZ¹¹—C(═O)—C₁₋₆ alkylene-, —C(═O)—NZ¹¹—C₁₋₆ alkylene-, —NZ¹¹—C(═O)—NZ¹¹—C₁₋₆ alkylene-, —NZ¹¹—C(═O)—O—C₁₋₆ alkylene-, —S—C₁₋₆ alkylene-, or —NZ¹¹—S(═O)₂—C₁₋₆ alkylene-. Z⁵ can be —NZ¹¹—C₁₋₆ alkylene-, —O—C₁₋₆ alkylene-, —NZ¹¹—C₁₋₆ alkylene-, —NZ¹¹—C(═O)—C₁₋₆ alkylene-, —NZ¹¹—C(═O)—NZ¹¹—C₁₋₆ alkylene-, —NZ¹—C(═O)—O—C₁₋₆ alkylene-, —S—C₁₋₆ alkylene-, or —NZ¹¹—S(═O)₂—C₁₋₆ alkylene-. Z⁵ can be —C(═O)—NZ¹¹—C₁₋₆ alkylene-.
Z⁶ can be H, —C(═O)—C₁₋₆ alkyl, —C(═O)—O—C₁₋₆ alkyl, —C(═O)—N(Z¹¹)₂, —P(═O)(Z¹¹)₂, or —P(═O)(OZ¹¹)₂. Z⁶ can be H.
Z⁷ can be —C(═O)—, —C(═O)—NZ¹¹—, —C(═O)—NZ¹¹—C₁₋₆ alkylene-, —S(═O)₂—, —S(═O)₂—NZ¹¹—.
Z⁸ can be a covalent bond, —O—, —C(═O)—, —C(═S)—, —NZ¹¹—, —S(═O)—, or —S(═O)₂—.
Z⁹ can be —C₅₋₆ arylene- or —C₃₋₅ heteroarylene-.
Z¹⁰ can be —C₅₋₆ arylene- or —C₃₋₅ heteroarylene-. Z¹⁰ can optionally be substituted with —C₁₋₆ alkyl.
Z¹¹ can be H or —C₁₋₆ alkyl.

The ubiquitin ligase binding moiety can have the formula:

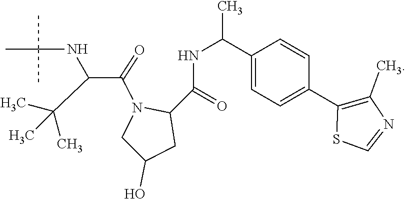

The Linker can be designed and optimized based on SAR (structure-activity relationship) and X-ray crystallography of the Targeting Moiety with regard to the location of attachment for the Linker.

The optimal Linker length and composition can vary by target and can be estimated based upon X-ray structures of the original Targeting Moiety bound to its target. Linker length and composition can be also modified to modulate metabolic stability and pharmacokinetic (PK) and pharmacodynamics (PD) parameters.

Where the Targeting Moiety binds multiple targets, selectivity can be achieved by varying Linker length where the ligand binds some of its targets in different binding pockets, e.g., deeper or shallower binding pockets than others.

The Linker (“T”) provides a covalent attachment between the Targeting Moiety and the Degron. The Linker has two terminating groups, wherein one terminating group attaches to the Degron and the other terminating group attaches to the Targeting Moiety. The structure of the Linker may not be critical, provided it does not substantially interfere with the activity of the Targeting Moiety or the Degron. The Linker can be an alkylene chain (e.g., having 2-20 alkylene units), or a polyethylene glycol (PEG) chain (CH₂CH₂—O or (O—CH₂CH₂)). The Linker can be an alkylene chain, a PEG chain, or a bivalent alkylene chain, any of which can be interrupted by, and/or terminate (at either or both termini) by at least one of the following: —O—, —S—, —N(T⁷)-, —C≡C—, —C(═O)—, —C(═O)O—, —OC(═O)—, —OC(═O)O—, —C(NOT⁷)-, —C(═O)N(T⁷)-, —C(═O)N(T⁷)C(═O)—, —C(═O)N(T⁷)C(═O)N(T⁷)-, —N(T⁷)C(═O)—, —N(T⁷)C(═O)N(T⁷)-, —N(T⁷)C(═O)O—, —OC(═O)N(T⁷)-, —C(NT⁷)-, —N(T⁷)C(NT⁷)-, —C(NT⁷)N(T⁷)-, —N(T⁷)C(NT⁷)N(T⁷)-, —OB(CH₃)O—, —S(═O)₂—, —OS(═O)—, —S(═O)O—, —S(═O)—, —OS(═O)₂—, —S(═O)₂O—, —N(T⁷)S(═O)₂—, —S(═O)₂N(T⁷)-, —N(T⁷)S(═O)—, —S(═O)N(T⁷)-, —N(T⁷)S(═O)₂N(T⁷)-, —N(T⁷)S(═O)N(T⁷)-, —C₃₋₁₂ cycloalkylene-, -3- to 12-membered heterocycloalkylene-, -5- to 12-membered heteroarylene-, or -arylene-, or any combination thereof, wherein T⁷ is H or —C₁₋₆ alkyl, wherein the interrupting and the one or both terminating groups may be the same or different.

The Linker can be C₁₋₁₀ alkylene chain terminating in NH-group wherein the nitrogen is also bound to the Degron, or the Linker can be a C₁₋₁₀ alkylene chain or a PEG chain having 1-8 PEG units and interrupted by or terminating in —(CH₂)_{n′}—C(O)—NH—, where n′ is 0 to about 5.

Nonlimiting examples of a Linker include —(CH₂CH₂—O)_{n″}—(CH₂)_{n′}—C(═O)—, —(CH₂)_{n′}—C(═O)—N(T⁷)-, —(CH₂CH₂—O)_{n′}—(CH₂)_{n′}—C(═O)—, —(CH₂CH₂—O)_{n′}—(CH₂)_{n′}—N(T7)-C(═O)—, —(CH₂CH₂—O)_{n″}—(CH₂)_{n′}—C(═O)—N(T⁷)-, —(CH₂)_{n′}-phenylene-N(T⁷)-C(═O)—(CH₂)_{n′}—, —N(T⁷)-(CH₂)_{n′}—O-phenylene-(CH₂)_{n″}—N(T⁷)-(CH₂)_{n′}—, —(CH₂)_{n′}—C(═O)—N(T⁷)-phenylene-C(═O)—, —N(T⁷)-(CH₂)_{n′}-phenylene-(CH₂)_{n′}-heterocycloalkylene-, —(CH₂)_{n′}-phenylene-N(T⁷)-C(═O)—(CH₂CH₂—O)_{n″}—(CH₂)_{n′}—, —(CH₂)_{n′}-phenylene-(CH₂)_{n′}-heterocycloalkylene-(CH₂)_{n′}—C(═O)—N(T⁷)-(CH₂)_{n′}—, —(CH₂)_{n′}-phenylene-O—(CH₂)_{n′}-heterocycloalkylene-(CH₂)_{n′}—, —(CH₂)_{n′}-phenylene-(CH₂)_{n′}-heterocycloalkylene-(CH₂)_{n′}—O—, or —(CH₂)_{n′}-heterocycloalkylene-(CH₂)ₙ. The variable n′ can be 0 to 10 and the variable n″ can be 1 to 10.

The Linker can have the formula -T¹-, -T¹-T²-, -T¹-T³-, -T²-T³-, or -T¹-T²-T³. The Linker can have the formula -T¹-, -T¹-T²-, -T¹-T³-, or -T¹-T²-T³. The Linker can have the formula -T¹-T²-T³-. T¹ can be —C₀₋₃ alkylene-C≡C—, —C₁₋₆ alkoxy-, —C₁₋₆ alkylene-, —C₀₋₃ alkylene-C₃₋₉ heterocyclylene-, —C₀₋₃ alkylene-C₅₋₆ arylene-; wherein the —C₃₋₉ heterocyclylene- is optionally substituted with halogen or —OH and can optionally contain 1 to 3 heteroatoms, for example, N. T¹ can be —C₁₋₃ alkylene-, —C₀₋₃ alkylene-C₃₋₅ heterocyclylene-, or —C₀₋₃ alkylene-C₅s arylene-; wherein the —C₃₋₅ heterocyclylene- is optionally substituted with halogen or —OH. T¹ can be piperidinylene or piperazinylene T¹ can be —C(═O)—, —C₁₋₆ alkoxy-, —C₁₋₆ fluoroalkoxy-, —C₁₋₆ alkylene-, —C₁₋₆ fluoroalkylene-, —C₀₋₃ alkylene-C₅₋₆ arylene-, —C₀₋₃ alkylene-C₄₋₅ heteroarylene-, —C₀₋₃ alkylene-C₃₋₁₀ cycloalkylene-C₀₋₃ alkylene, —C₀₋₃ alkylene-C₃₋₉ heterocycloalkylene-C₀₋₃ alkylene-, —C₀₋₃ alkylene-O—C₃₋₉ heterocycloalkylene-C₀₋₃ alkylene-, —C₁₋₃ alkylene-C≡C—, —C₁₋₃ alkylene-, —C₁₋₃ alkylene-O—. T¹ can be —C₁₋₆ alkoxy-, —C₁₋₆ alkylene-, —C₀₋₃ alkylene-C₃₋₈ cycloalkylene-C₀₋₃ alkylene-, or —C₀₋₃ alkylene-C₃₉ heterocycloalkylene-C₀₋₃ alkylene-. The Linker can have the formula -T¹- and T¹ can be —C₀₋₃ alkylene-C₃₋₈ heterocycloalkylene-C₀₋₃ alkylene- or C₁₋₆ alkylene. T¹ can be piperidinyl, pyridinyl, pyrimidinyl, pyridazinyl, pyrazinyl, pyrazolyl, piperazinyl, quinoxalinyl, phenyl, triazolyl, thiazolyl, thiadiazolyl, oxazolyl, imidazolyl, indazolyl, —C₁₋₆ alkylene, —C₁₋₆ fluoroalkylene, —C₁₋₆ alkoxy, pyrrolopyridinyl, isoindolinyl, isoquinolinyl, tetrahydroisoquinolinyl, thiazolopyridinyl, tetrahydrothiazolopyridinyl, imidazopyrazinyl, tetrahydroimidazopyrazinyl, pyrazolopyrazinyl, tetrahydropyrazolopyrazinyl, phenyl, oxadiazaspirodecanyl, diazaspirooctanyl, or diazaspirodecan-1-one. T¹ can be piperidinyl or pyridazinyl. T¹ can optionally be substituted with one or two T⁴. T¹ can optionally be substituted with one or two halogen, oxo, —OH, —C₁₋₆ alkyl, —C₃₋₆ cycloalkyl, —C₁₋₆ fluoroalkyl, —C₁₋₆ alkoxy, or —C₃₋₆ cycloether. The respective heteroarylene- or -heterocycloalkylene- groups of T¹ can have 1, 2, or 3 heteroatoms selected from O, N, and S.

T² can be —O—, —NR¹⁰—, —C₃₋₆ cycloalkylene-, —C₀₋₃ alkylene-C₅₋₆ arylene-, or —C₁₋₆ alkylene-O—. T² can be —O—, —NR¹⁰—, —C₃₋₆ cycloalkylene-, or —C₀₋₃alkylene-C₅₋₆ arylene-. T² can be is —O—, —NH—C(═O)-T⁸-, —S(═O)₂-T³-, —O—S(O)₂-T³-, —C(═O)—, —NT⁷-, —C₃₋₆ cycloalkylene-, —C₃₋₅ heterocycloalkylene-, —C₁₋₆ alkylene-, —C₁₋₆ alkylene-O—, —C₁₋₆ aminoalkylene-, —C₁₋₆ fluoroalkylene-, —C₁₋₆ alkoxy-, —C₁₋₆ fluoroalkoxy-, —C₃₋₆ cycloether-, —C₅₋₆ arylene, or —C₄₋₅ heteroarylene-. T² can be —O—, —C₃₋₆ cycloalkylene-, —C₅₋₆ arylene, —C₁₋₆ alkylene-, or —C₁₋₆ alkylene-O—. T² can be —O—, —NT⁷-, —C₅₋₆ arylene, or —C₄₋₅ cycloalkylene-. T² can be —C(═O)—, —C₁₋₃ alkylene, —C₁₋₆ aminoalkylene, —C₁₋₆ alkoxy, pyridinyl, oxolanyl, —C₃₋₆ cycloalkyl, —C₁₋₆ fluoroalkylene, piperazinyl, piperidinyl, azetidinyl, azaspiroundecanyl, azaspirononanyl, azaspiroundecanyl, diazaspirooctanyl, diazaspirodecanyl, diazaspirononanyl, diazaspirododecanyl, diazaspiroundecanyl, oxadiazaspirononanyl, oxadiazaspiroundecanyl, oxa-azaspirodecanyl, decahydronaphthyridinyl, octahydropyrrolopyridinyl, or octahydropyridopyrazinyl. T² can be absent, —O—, —C₁₋₆ alkylene-, or —C₁₋₆ alkylene-O—. T² can optionally be substituted with one, two or three T⁵. T² can optionally substituted with one, two or three halogen, oxo, —OH, —C₁₋₆ alkyl, —C₃₋₆ cycloalkyl, —C₁₋₆ fluoroalkyl, or —C₁₋₆ alkoxy. The respective heteroarylene or heterocycloalkylene groups of T² can each independently have 1, 2, or 3 heteroatoms selected from O, N, and S.

T³ can be —C₁₋₆ alkoxy-, —C₁₋₆ alkylene-, —C₀₋₃ alkylene-C═C—C₀₋₃ alkylene-, —C₁₋₃ alkylene-O—, —C₁₋₃ alkylene-O—C₁₋₃ alkylene-C(═O)—C₃₋₈ heterocyclylene-, —C₁₋₃ alkylene-O—C₁₋₃ alkylene-C(═O)—NH—, —C₀₋₆ alkylene-NR¹⁰-C₀₋₆ alkylene-, —C₀₋₆ alkylene-C₃₋₈ heterocyclylene-C₀₋₆ alkylene-, or —C₀₋₆ alkylene-O—C₃₋₈ heterocyclylene-C₀₋₆ alkylene-; wherein the respective C₃₋₈ heterocyclylenes are optionally substituted with 1 or 2 halogen. T³ can be —C₁₋₃ alkylene-, —C₁₋₃ alkylene-C≡C—, —C≡C—C₁₋₃ alkylene-, —C₁₋₃ alkylene-O—, —C₁₋₃ alkylene-O—C₁₋₃ alkylene-C(═O)—C₃₋₈ heterocyclylene-, —C₁₋₃ alkylene-O—C₁₋₃ alkylene-C(═O)—NH—, —C₀₋₆ alkylene-NR¹⁰-C₀₋₃ alkylene-, —C₀₋₃ alkylene-C₃₋₈ heterocyclylene-C₀₋₃ alkylene-, or —C₀₋₃ alkylene-O—C₃₋₈ heterocyclylene-C₀₋₃ alkylene-; wherein the respective C₃₋₈ heterocyclylenes include 1 to 3 heteroatoms and are optionally substituted with 1 or 2 halogen. T³ can be —C₁₋₆ alkoxy-, —C(═O)—, —C₁₋₆ alkylene-, —C₁₋₆ aminoalkylene-, —NH—C₁₋₆ alkylene-, —C(═O)—C₁₋₆ alkylene-, —C₁₋₆ alkylene-C≡C—, —C₁₋₃ alkylene-O—, —C₀₋₆ alkylene-C₃₋₈ heterocycloalkylene-C₀₋₆ alkylene-, —C₀₋₆ alkylene-O—C₃₋₈ heterocycloalkylene-C₀₋₆ alkylene-, —C₀₋₆ alkylene-C₃₋₈ heterocycloalkylene-C(═O)—, —C₅₋₆ arylene-, —C₃₋₈ heteroalkylene-C₁₋₆ alkylene-C₅₋₆ arylene-C₁₋₆ alkoxy-, —C₁₋₆ heteroalkylene-C₁₋₆ heterocycloalkylene-C(═O)—, —O—C₁₋₆ heterocyclylalkylene-C(═O)—, —C₁₋₆ cycloalkylene-C₁₋₆ heterocycloalkylene-, —C₀₋₂ alkylene-C₅₋₆ arylene-C₁₋₆ alkoxy-, or —C₄₋₅ heteroarylene-. T³ can be —C₀₋₃ alkylene-C₃₋₈ heterocycloalkylene-C₀₋₃ alkylene-, —C₀₋₃ alkylene-O—C₃₋₈ heterocycloalkylene-C₀₋₃ alkylene-, —C₁₋₃ alkylene-C≡C—, —C₁₋₃ alkylene-, —C₁₋₃ alkylene-O—. T³ can be —C₁₋₆ alkylene-, —C₁₋₆ aminoalkylene-, —C₁₋₆ alkoxy-, —C(═O)—, —C₀₋₆ alkylene-heterocyclyl-C(═O)—, —C(═O)—C₁₋₆ alkylene-, -heterocyclyl-C₁₋₆ alkylene-aryl-C₁₋₆ alkoxy-, —C₁₋₆ heterocyclyl-C₁₋₆ heterocyclyl-C(═O)—, —C₀₋₂ alkylene-aryl-C₁₋₆ alkoxy-, —O-heterocyclyl-C(═O)—, —C₁₋₆ cycloalkyl-C₁₋₆ heterocyclyl. T³ can be —C₁₋₆ alkylene, —C₁₋₆ alkylene-C≡C—, or —C₀₋₆ alkylene-C₃₋₈ heterocycloalkylene-C₀₋₆ alkylene-. T³ can optionally be substituted with one, two, or three T⁶. The C₃₋₈ heterocycloalkylene of T³ can optionally be substituted with one, two, or three of F. The respective heteroarylene- or -heterocycloalkylene- groups of T³ can have 1, 2, or 3 heteroatoms selected from O, N, and S.

If T² is —O—, then T³ is not —C₀ alkylene-O—C₃₋₈ heterocycloalkylene-C₀₋₆ alkylene- or —Co alkylene-NR¹⁰—.

T¹ can be —C₀₋₃ alkylene-C₃₋₈ heterocycloalkylene-; T² can be absent, —O—, —C₁₋₆ alkylene-, or —C₁₋₆ alkylene-O—; and T³ can be —C₁₋₆ alkylene, —C₁₋₆ alkylene-C≡C—, or —C₀₋₆ alkylene-C₃₋₈ heterocycloalkylene-C₀₋₆ alkylene-.

T can have the formula -T¹-, -T¹-T²-, -T¹-T³-, -T²-T³-, or -T¹-T²-T³-; wherein T¹ can be —C₀₋₅ alkylene-C₃₋₁₀ heterocycloalkylene-; T² can be —O—, —C₁₋₆ alkylene-, —C₁₋₆ alkylene-O—, or —C₀₋₆ alkylene-NR¹⁰—; and T³ can be —C₁₋₆ alkylene-, —C₁₋₆ alkylene-C≡C—, —C₀₋₆ alkylene-C₃₋₉ heterocycloalkylene-C₀₋₆ alkylene-, —C₀₋₆ alkylene-C₃₋₉ heterocycloalkylene-C₁₋₆ alkylene-O—, —C₀₋₆ alkylene-C₃₋₉ cycloclylene-C₀₋₆ alkylene-, —C₀₋₆ alkylene-C₃₋₉ cycloclylene-C₁₋₆ alkylene-O—.

T can have the formula -T¹-, -T¹-T²-, -T¹-T³-, or -T¹-T²-T³-; wherein T¹ is —C₀₋₅ alkylene-C₃₋₁₀ heterocycloalkylene-; T² is —O—, or —C₀₋₆ alkylene-NR¹⁰—; and T³ is —C₁₋₆ alkylene-, —C₁₋₆ alkylene-C≡C—, —C₀₋₆ alkylene-C₃₋₉ heterocycloalkylene-C₀₋₆ alkylene-, —C₀₋₆ alkylene-C₃₋₉ heterocycloalkylene-C₁₋₆ alkylene-O—, —C₀₋₆ alkylene-C₃₋₉ cycloclylene-C₀₋₆ alkylene-, —C₀₋₆ alkylene-C₃₋₉ cycloclylene-C₁₋₆ alkylene-O—.

T can have the formula -T¹-, -T¹-T²-, -T¹-T³-, or -T¹-T²-T³-; wherein T¹ is —C₀₋₃ alkylene-C≡C—, —C₁₋₆ alkoxy-, —C₁₋₆ alkylene-, —C₀₋₃ alkylene-C₃₋₉ heterocyclylene-, —C₀₋₃ alkylene-C₅₋₆ arylene-; for example, piperidinylene or piperazinylene; wherein the —C₃₋₉ heterocyclylene- is optionally substituted with halogen or —OH; T² is —O—, —NR¹⁰—, —C₃₋₆ cycloalkylene-, —C₀₋₃ alkylene-C₅₋₆ arylene-, or —C₁₋₆ alkylene-O—; wherein if T² is —O—, then T³ is not —Co alkylene-O—C₃₋₈ heterocycloalkylene-C₀₋₆ alkylene- or —Co alkylene-NR¹⁰—; and wherein T³ is —C₁₋₆ alkoxy-, —C₁₋₆ alkylene-, —C₀₋₃ alkylene-C≡C—C₀₋₃ alkylene-, —C₁₋₃ alkylene-O—, —C₁₋₃ alkylene-O—C₁₋₃ alkylene-C(═O)—C₃₋₈ heterocyclylene-, —C₁₋₃ alkylene-O—C₁₋₃ alkylene-C(═O)—NH—, —C₀₋₆ alkylene-NR¹⁰-C₀₋₆ alkylene-, —C₀₋₆ alkylene-C₃₋₈ heterocyclylene-C₀₋₆ alkylene-, or —C₀₋₆ alkylene-O—C₃₋₈ heterocyclylene-C₀₋₆ alkylene-; wherein the respective C₃₋₈ heterocyclylenes of T³ are optionally substituted with 1 or 2 halogen.

T can have the formula -T¹-, -T¹-T²-, -T¹-T³-, or -T¹-T²-T³-; wherein T¹ is —C₁₋₃ alkylene-, —C₀₋₃ alkylene-C₃₋₈ heterocyclylene-, or —C₀₋₃ alkylene-C₅₋₆ arylene-; for example, piperidinylene or piperazinylene; wherein the —C₃₋₅ heterocyclylene- is optionally substituted with halogen or —OH; T² is —O—, —NR¹⁰—, —C₃₋₆ cycloalkylene-, or —C₀₋₃ alkylene-C₅₋₆ arylene-; wherein if T² is —O—, then T³ is not —C₀ alkylene-O—C₃₋₈ heterocycloalkylene-C₀₋₆ alkylene- or —Co alkylene-NR¹⁰—; and T³ is —C₁₋₃ alkylene-, —C₁₋₃ alkylene-C≡C—, —C≡C—C₁₋₃ alkylene-, —C₁₋₃ alkylene-O—, —C₁₋₃ alkylene-O—C₁₋₃ alkylene-C(═O)—C₃₋₈ heterocyclylene-, —C₁₋₃ alkylene-O—C₁₋₃ alkylene-C(═O)—NH—, —C₀₋₆ alkylene-NR¹⁰-C₀₋₃ alkylene-, —C₀₋₃ alkylene-C₃₋₈ heterocyclylene-C₀₋₃ alkylene-, or —C₀₋₃ alkylene-O—C₃₋₈ heterocyclylene-C₀₋₃ alkylene-; wherein the respective C₃₋₈ heterocyclylenes include 1 to 3 heteroatoms and are optionally substituted with 1 or 2 halogen.

T⁴, T⁵, and T⁶ can each independently be oxo, —OH, halogen, —C₁₋₆ alkyl, —C₁₋₆ alkoxy, —C₁₋₆ fluoroalkyl, —C₃₋₆ cycloalkyl, heteroaryl, or heterocyclyl. The heteroaryl and the heterocyclyl of each of T⁴, T⁵, and T⁶ can each independently have 1, 2, or 3 heteroatoms selected from O and N.

T⁷ can be H or C₁₋₆ alkyl.

T³ can be —C₁₋₆ alkyl, —C₁₋₆ alkoxy, —C₁₋₆ fluoroalkyl, or —C₃₋₆ cycloalkyl.

The targeting moiety can have the structure of Formula M or M-I.

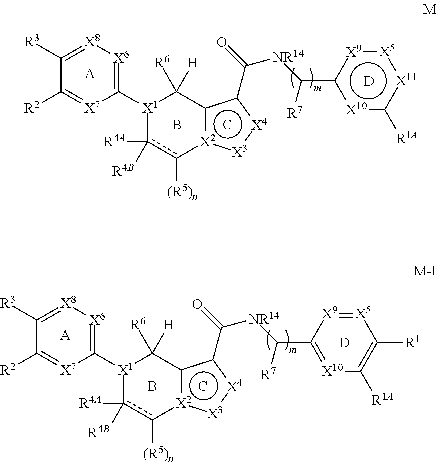

X¹ can be CH, CNH₂, or N. X¹ can be N.
X² can be C or N.
X³ can be CR¹³, N, NR¹³, O, or S. X³ can be CR¹³. X³ can be N or NR¹³. X³ can be CR¹³ or N.
X⁴ can be CH, N, or NR¹³. X⁴ can be CH, N, or N-cyclopropyl. X⁴ can be N.
X⁵ can be CH, CR^{1B}, or N. X⁵ can be CH.
X⁶ can be CH or N.
X⁷ can be CH, N, R^{2A}, or CF. X⁷ can be CH or N. X⁷ can be CCI.
X⁸ can be CH or N. X⁸ can be CH.
X⁹ can be CH, CR^{1B}, or N.
X¹⁰ can be CH, CR¹B, or N.
X¹¹ can be CR¹, N, or NCH₃.

At least one of X² can be N or X³ can be N, NH, or NR¹³, and X⁴ can be N.

If X³ is S or O, then X² can be C.

If X³ is CR¹³ or NR¹³, then X⁴ can be CH or N. If X⁴ is NR¹³, then X³ can be N, O, or S.

The dashed line refers to the bond being a single bond (i.e., the dashed line is not present leaving only the single bond) or a double bond (i.e., the dashed line is present as a second bond making the double bond). The dashed line can refer to the bond being a single bond. If the dashed line refers to the bond being a double bond, then R^{4B} is absent and n is 1.

R¹ can be divalent and can be —NR¹⁰—C(═O)—, —C(═O)—NR¹⁰—, or —NR¹⁰—C(═O)—NR¹⁰—. R¹ can be —NR¹⁰—C(═O)—, —NR¹⁰—C(═O)—NR¹⁰—, H, —C(═O)—NR¹⁰—, —NHR⁸, —OH, —C₁₋₄ hydroxyalkyl, —C₁₋₃ fluoroalkyl, —C₀₋₁ alkylene-C₂₋₆ cyclyl, —C(═O)—NR¹⁰R⁸, —C(═O)—NR¹⁰-C₀₋₁ alkylene-phenyl, —C₀₋₁ alkylene-C₁₋₅ heterocyclyl, —O—C₁₋₅ heterocyclyl, or —C₁₋₅ alkyl. R¹ can be monovalent and can be H, —NHR⁸, —OH, —C₂₋₅ heterocyclyl, or —C₁₋₃ alkyl. R¹ can be —NHR⁸, —C₂₋₅ heterocyclyl, or —C₁₋₃ alkyl. R¹ can be —C₁₋₅ heterocyclyl or —C₂₋₅ heterocyclyl and can optionally be substituted with one or more or one, two, or three of —C₁₋₃ alkyl, halogen, —C₁₋₃ oxoalkyl, —C₃₋₅ heterocycloalkyl, —C₃₋₅ hydroxyheterocycloalkyl, —C₃₋₅ aminoheterocycloalkyl, or oxo. R¹ can be —C₂₋₅ heterocyclyl and can optionally be substituted with one or more or one, two, or three of —C₁₋₃ alkyl or oxo. R¹ can be —C₂₋₅ heterocyclyl and can optionally be substituted with oxo. R¹ can be —C₁₋₃ alkyl and can be substituted with one, two, or three of oxo, —NR^{10A}R¹¹, or —NR¹⁰R¹¹. R¹ can be —C₁₋₃ alkyl optionally substituted with one or more or one, two, or three of oxo or —NR¹⁰R¹¹. R¹ can be —C₁₋₃ alkyl substituted with oxo and —NR¹⁰R¹¹. R¹ can be —NHR⁸. Each of the —C₂₋₅ heterocyclyl, —C₃₋₅ heterocycloalkyl, —C₃₋₅ hydroxyheterocycloalkyl, and the —C₃₋₅ aminoheterocycloalkyl independently can include 1 to 4 heteroatoms including at least one of N, O, or S. The —C₂₋₅ heterocyclyl of R¹ can include 1 to 4 heteroatoms including at least one of N, O, or S; where the heteroatoms can all be N.

R^{1A} can be H. R^{1A} can be —CH₃. R^{1A} can be H, halogen, —C₂₋₃ heterocyclyl, oxo, —C(═O)NR¹⁰R¹¹; or —CH₃, wherein the —C₂₋₃ heterocyclyl can optionally be substituted with —C₁₋₃ alkyl. R^{1A} can be H or halogen, where the halogen can be F.

R¹ and R^{1A} taken together can form a C₄₋₇ heterocycloalkyl fused to Ring D or a C₄₋₇ heteroaryl fused to Ring D. R¹ and R^{1A} taken together can form a C₅₋₇ heterocycloalkyl fused to Ring D or a C₅₋₇ heteroaryl fused to Ring D. The C₄₋₇ heterocycloalkyl or the C₄₋₇ heteroaryl can optionally be substituted with one or two oxo or —C₂₋₃ oxoalkyl. The C₅₋₇ heterocycloalkyl or the C₅₋₇ heteroaryl can optionally be substituted with oxo. R¹ and R^{1A} can taken together form a C₆-8 fused heterocycloalkyl that is optionally substituted with oxo. R¹ and R^{1A} taken together can form a C₆₋₈ fused heteroaryl that is optionally substituted with oxo. Each of the C₆₋₈ fused heterocycloalkyl and the can C₆₋₈ fused heteroaryl independently can include 1 to 3 heteroatoms including at least one of N, O, or S.

Each R^{1B} independently is H, —CH₃, F, Cl, or methoxy.

R² can be H, —C₁₋₃ alkyl, —C₁₋₃ alkoxy, —C₁₋₃ fluoroalkyl, or halogen. R² can be H.

R² and R^{2A} can be taken together to form a six membered heteroaryl fused to Ring A.

R³ can be divalent and can be —C(═O)—, —C(═O)—NR^{12A}—, —NR^{12A}—C(═O)—, or C₂₋₇ heterocyclene-. R³ can be monovalent and can be —C₁₋₃ alkyl, —C₁₋₁₀ heterocyclyl, —P(═O)(CH₃)₂, —S(═O)CH₃, —NH—S(═O)₂CH₃, or —NH—C(═O)—C₁₋₃ alkyl. R³ can be —C₁₋₃ alkyl, —C₂₋₁₀ heterocyclyl, —P(═O)(CH₃)₂, —S(═O)CH₃, or —NH—S(═O)₂CH₃. R³ can be —C₁₋₃ alkyl, —C₂₋₁₀ heterocyclyl, or —NH—C(═O)—C₁₋₃ alkyl. R³ can be —C₁₋₃ alkyl optionally substituted with one or more or one, two, or three of oxo, —OH, halogen, —C₃₋₇ cycloalkyl, —NR¹²R^{12A}, —C₅₋₈ heterocyclyl, or cyano. R³ can be —C₁₋₃ alkyl substituted with oxo and —NR¹²R^{12A}. The —C₁₋₁₀ heterocyclyl of R³ can be —C₁₋₁₀ heterocycloalkyl or —C₁₋₁₀ heteroaryl. The —C₁₋₁₀ heterocyclyl of R³ can be —C₂₋₆ heterocycloalkyl or —C₂₋₆ heteroaryl. R³ can be —C₁₋₁₀ heterocyclyl optionally substituted with one or more or one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo. R³ can be —C₁₋₁₀ heterocycloalkyl optionally substituted with one or more or one, two, or three of —OH, halogen, —C₁₋₃ hydroxyalkyl, —C₁₋₃ alkyl, cyclopropyl, or oxo. R³ can be —C₁₋₃ alkyl optionally substituted with one or more or one, two, or three of oxo or —NR¹²R^{12A}. R³ can be —C₂₋₅ heterocyclyl optionally substituted with —C₁₋₃ alkyl. R³ can be —C₂₋₅ heteroaryl optionally substituted with one or more or one, two, or three —C₁₋₃ alkyl. Each of the —C₁₋₁₀ heterocyclyl, —C₆₋₈ heterocyclyl, —C₂₋₆ heterocycloalkyl, —C₁₋₁₀ heterocycloalkyl, —C₁₋₁₀ heteroaryl, —C₂₋₆ heteroaryl, or —C₂₋₅ heterocyclyl independently can include 1 to 3 heteroatoms including at least one of N, O, or S. The —C₁₋₁₀ heterocyclyl of R³ can include 1 to 3 heteroatoms including at least one of N, O, or S. The —C₁₋₁₀ heterocyclyl of R³ can be a spiro, fused, or bridged —C₂₋₁₀ heterocyclyl. The C₅₋₈ heterocyclyl of R³ can include 1 to 4 heteroatoms including at least one of N, O, or S.

R² and R³ taken together can form a C₃₋₆ heterocyclyl fused to Ring A optionally substituted with one, two, or three of —C₁₋₃ alkyl, —C₂₋₃ oxoalkyl, or oxo. R² and R³ taken together can form a C₃₋₆ heterocycloalkyl fused to Ring A optionally substituted with one or more or one, two, or three of —C₁₋₃ alkyl, —C₂₋₃ oxoalkyl, or oxo. Each of the C₃₋₆ heterocyclyl or the C₃₋₆ heterocycloalkyl independently can include 1 to 3 heteroatoms including at least one of N, O, or S.

R^{4A} can be H, —OH, —C₁₋₃ fluoroalkyl, or —C₁₋₃ alkyl. R^{4A} can be H, —C₁₋₃ fluoroalkyl, or —C₁₋₃ alkyl.

R^{4B} can be H. R^{4B} can be absent.

R^{4A} and R^{4B} taken together can form cyclopropyl.

R^{4B} and one R⁵ taken together can form a C₃₋₅ cycloalkyl fused to Ring B.

Each R⁵ independently can be H, —OH, —C₃₋₆ cycloalkyl (e.g., cyclopropyl), halogen, —C₁₋₃ fluoroalkyl, or —C₁₋₃ alkyl. Each R⁵ independently can be H, halogen, or —C₁₋₃ alkyl. The two R⁵ groups taken together can form a C₃₋₄ cycloalkyl or a C₃₋₅ heterocycloalkyl containing 1 or two N atoms. The two R⁵ groups taken together can form cyclopropyl.

R⁶ can be H, —OH, or —C₁₋₃ alkyl. R⁶ can be H or —C₁₋₃ alkyl.

R^{4A} and R⁶ taken together can form a C₁₋₃ alkyl bridge or a C₁₋₃ heteroalkyl bridge. One R⁵ and R⁶ taken together can form a C₁₋₃ alkyl bridge or a C₁₋₃ heteroalkyl bridge. The C₁₋₃ heteroalkyl bridge can include one heteroatom of N, O, or S.

R⁷ can be H, —C₁₋₃ alkyl, or —C₁₋₃ hydroxyalkyl. R⁷ can be H or —C₁₋₃ alkyl.

R⁸ can be H, —C₁₋₃ alkyl, —SO₂CH₃, or a —C₂₋₅ heterocyclyl. R³ can be H, —C₁₋₃ alkyl, or a —C₃₋₄ heterocyclyl. R³ can be the —C₁₋₃ alkyl substituted with one, two, or three of oxo, —C₃₋₉ heterocyclyl, —C₃₋₉ heterocyclyl substituted with methyl, —C₁₋₃ alkoxy, cyanoimine, —C₅₋₆ aryl, —O—C₅₋₆ aryl, or —NR⁹R¹⁰. R³ can be the —C₁₋₃ alkyl optionally substituted with one or more or one, two, or three of oxo or —NR⁹R¹⁰. R⁸ can be the —C₁₋₃ alkyl substituted with oxo and —NR⁹R¹⁰. R⁸ can be the —C₂₋₅ heterocyclyl optionally substituted with one, two, or three of oxo, halogen, —C₀₋₁ alkylene-NR¹⁰R¹¹, —OH, —C₁₋₃ hydroxyalkyl, —C₁₋₃ alkyl-C₁₋₃ alkoxy, —C₁₋₃ oxoalkyl, —C₁₋₃ alkyl, or —C₁₋₃ alkoxy optionally substituted with —NR¹⁰R¹¹. The —C₃₋₉ heterocycloalkyl of R³ can be substituted with one, two, or three of —C₁₋₃ alkyl, —OH, —C₁₋₃ hydroxyalkyl, —O—C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃-s heterocycloalkyl, —O—C₃₋₅ heterocycloalkyl, phenyl optionally substituted with halogen, or —C₀₋₁ alkylene-NR¹⁰R¹¹. R³ can be the —C₃₋₄ heterocyclyl optionally substituted with one or more or one, two, or three of oxo or —C₁₋₃ alkyl. Each of the —C₃₋₄ heterocyclyl or the —C₃₋₉ heterocycloalkyl of R³ independently can include 1 to 4 heteroatoms including at least one of N, O, or S. The —C₃₋₄ heterocyclyl of R³ can include 1 to 4 heteroatoms including at least one of N or O. The —C₃₋₉ heterocycloalkyl of R³ can include 1 to 3 heteroatoms including at least one of N or O. The —C₃₋₄ heterocyclyl or the —C₃₋₉ heterocycloalkyl of R³ can be a bridged, spiro, or fused heterocyclyl. The —C₅₋₆ aryl can optionally be substituted with a C₄₋₅ heterocycloalkyl or halogen.

Each R⁹ independently can be H, —C₁₋₅ alkyl, —C₁₋₄ hydroxyalkyl, —C₁₋₃ fluoroalkyl, —C₃₋₅ cycloalkyl, —C₃₋₅ heterocycloalkyl, -methylene-phenylene-NH—C(═O)—NR¹⁰R¹¹, or —C₃-s heterocycloalkyl. R⁹ can be the —C₁₋₄ alkyl optionally substituted with one or more or one, two, or three of —NR¹⁰R¹¹, —C₃₋₅ cycloalkyl, —C₃₋₅ heterocycloalkyl, cyano, fluoromethoxy, or methoxy. The respective —C₃₋₅ cycloalkyl and —C₃₋₅ heterocycloalkyl groups of R⁹ are optionally substituted with one or two of —OH, oxo, halogen, —C₁₋₃ alkoxy, —C₁₋₃ alkyl-C₁₋₃ alkoxy, or —C₁₋₃ alkyl. R⁹ can be the —C₃₋₅ cycloalkyl optionally substituted with one or two of —OH or —C₁₋₃ alkyl. R⁹ can be H or —C₁₋₄ alkyl. Each of the —C₃₋₅ heterocycloalkyl of R⁹ independently can include 1 to 2 heteroatoms including at least one of N or O.

Each R¹⁰ independently can be H or —C₁₋₄ alkyl. Each R¹⁰ independently can be H or —C₁₋₃ alkyl.

Each R^{10A} independently can be H, —C₁₋₅ alkyl, —C₁₋₄ hydroxyalkyl, —C₁₋₃ fluoroalkyl, —C₃₋₅ cycloalkyl, —C₃₋₅ heterocyclyl, -methylene-phenylene-NH—C(═O)—NR¹⁰R¹¹, or —C₀₋₁ alkylene-C₃₋₅ heterocycloalkyl optionally substituted with —C₁₋₃ alkyl. The —C₁₋₄ alkyl of R^{10A} is optionally substituted with one, two, or three of —NR¹⁰R¹¹, —C₃₋₅ cycloalkyl, —C₃₋₅ heterocycloalkyl, cyano, fluoromethoxy, or methoxy. The respective —C₃₋₅ cycloalkyl and —C₃₋₅ heterocycloalkyl groups of R^{10A} are optionally substituted with one or two of —OH, oxo, halogen, —C₁₋₃ alkoxy, —C₁₋₃ alkyl-C₁₋₃ alkoxy, or —C₁₋₃ alkyl. R^{10A} can be —C₀₋₁ alkylene-C₃₋₅ heterocycloalkyl optionally substituted with —C₁₋₃ alkyl. The C₃₋₅ heterocycloalkyl of R^{10A} can include 1 heteroatom of N.

If the —C₁₋₃ alkyl of R³ is substituted with —NR⁹R¹⁰, then the corresponding R⁹ and R¹⁰ can optionally be taken together to form the C₃₋₉ heterocycloalkyl or the C₃₋₉ heterocycloalkyl.

Each R¹¹ independently can be H or —C₁₋₃ alkyl.

R¹² can be H, —C₁₋₃ alkyl, —C₁₋₃ hydroxyalkyl, —(CH₂)₀₋₂—C₃₋₅ heterocycloalkyl, —(CH₂)₁₋₂ alkylene-O—C₃₋₅ heterocycloalkyl, -methylene-C(═O)—NR¹⁰R¹¹, or -methylene-phenylene-NH—C(═O)—NR¹⁰R¹¹. The —(CH₂)₀₋₂—C₃₋₅ heterocycloalkyl or the —(CH₂)₁₋₂ alkylene-O—C₃₋₅ heterocycloalkyl of R¹² can be substituted with —C₁₋₄ alkyl, —C₁₋₃ alkyl-C₁₋₃ alkoxy, —(CH₂)₀₋₁ alkylene-phenyl, halogen, —C₁₋₃ fluoroalkyl, or —(CH₂)₀₋₁ alkylene-C₃₋₅ heterocycloalkyl. The —C₁₋₃ alkyl of R¹² can be substituted with one, two, or three of oxo or NR¹⁰R¹¹. Each of the —(CH₂)₀₋₂—C₃-s heterocycloalkyl, the —(CH₂)₁₋₂ alkylene-O—C₃₋₅ heterocycloalkyl, or the —(CH₂)₀₋₁ alkylene-C₃₋₅ heterocycloalkyl of R¹² independently can include 1 to 2 heteroatoms including at least one of N or O.

R^{12A} can be H. R^{12A} can be —C₁₋₄ alkyl.

R¹² and R^{12A} taken together can form a C₃₋₁₀ heterocyclyl optionally substituted with one, two, or three of —C₁₋₃ alkyl, —OH, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR¹⁰R¹¹, or —NHC(═O)CH₃. The C₃₋₁₀ heterocyclyl can include 1 to 4 heteroatoms including at least one of N, O, or S. The C₃₋₁₀ heterocyclyl can be a bridged, spiro, or fused heterocyclyl.

R¹³ can be divalent and can be —C₁₋₆ alkylene- or —C(═O)—. R¹³ can be monovalent and can be H, —C₁₋₆ alkyl, —C₁₋₄ hydroxyalkyl, —C₁₋₃ fluoroalkyl, —C₁₋₃ alkylene-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃₋₅ heterocycloalkyl. R¹³ can be H, —C₁₋₄ alkyl, —C₁₋₄ hydroxyalkyl, —C₁₋₃ fluoroalkyl, —C₃₋₅ cycloalkyl, or —C₃₋₅ heterocycloalkyl. R¹³ can be H. R¹³ can be —C₀₋₁-cyclopropyl. The —C₁₋₆ alkyl of R¹³ can optionally be substituted with one, two, or three of oxo, fluoro, —NH₂, C₃₋₆ cycloalkyl, or methoxy. The —C₀₋₁ alkylene-C₃₋₅ cycloalkyl or the —C₀₋₁ alkylene-C₃₋₆ heterocycloalkyl of R¹³ can optionally be substituted with one, two, or three of —C₁₋₃ alkyl, —C₁₋₃ fluoroalkyl, or halogen. The —C₃₋₅ heterocycloalkyl of R¹³ can include 1 to 3 heteroatoms including at least one of N, O, or S.

R¹⁴ can be H. R¹⁴ can be —C₁₋₃ alkyl.

R¹⁵ is a bivalent connector, for example, a direct bond, —O—, —C₁₋₆ alkylene-, or —C(═O)—.

X⁴ can be NR¹³ and R¹³ and R¹⁴ taken together can form a C₄₋₅ heterocyclyl fused to Ring C.

X¹⁰ and R¹⁴ taken together can form a 5 to 6-membered heterocyclyl that is optionally substituted with C₁₋₃ alkyl and that is fused to ring D.

The variable n can be 1 or 2.

The variable m can be 0 or 1.

The respective heterocyclyl and heteroaryl rings can each independently include 1 to 4 heteroatoms including at least one of N, O, or S.

At least one hydrogen of the compound of Formula I can be deuterium.

In Formula M, one of R¹, R³, R¹³, or R¹⁵ can be a bivalent connector and is connected to T.

The compound or a pharmaceutically acceptable salt thereof can be any one of Examples E1 to E732 or any one of the virtual examples of Table 5.

In an aspect, the compound, or a pharmaceutically acceptable salt is (S)—N-(4-(3,3-Dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

In an aspect, the compound is (S)—N-(4-(3,3-Dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide.

The compound or a pharmaceutically acceptable salt thereof can be (7S)-5-(5-(dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide.

The compound or a pharmaceutically acceptable salt thereof can be (S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

The compound or a pharmaceutically acceptable salt thereof can be (S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide.

The compound or a pharmaceutically acceptable salt thereof can be (7S)—N-(4-(3,3-dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide.

The compound or a pharmaceutically acceptable salt thereof can be N-(4-(((S)-5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)-9-(3-(1-((RS)-2,6-dioxopiperidin-3-yl)-3-methyl-2-oxo-2,3-dihydro-1H-benzo[d]imidazol-5-yl)propyl)-3,9-diazaspiro[5.5]undecane-3-carboxamide.

The compound or a pharmaceutically acceptable salt thereof can be (S)-5-(5-(dimethylcarbamoyl)pyridin-2-yl)-N-(4-(4-((1-(3-(1-((RS)-2,6-dioxopiperidin-3-yl)-3-methyl-2-oxo-2,3-dihydro-1H-benzo[d]imidazol-5-yl)propyl)piperidin-4-yl)oxy)piperidine-1-carboxamido)benzyl)-1-isopropyl-7-methyl-4,5,6,7-tetrahydro-1H-pyrazolo[4,3-c]pyridine-3-carboxamide.

The compound or a pharmaceutically acceptable salt thereof can be (S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide.

The compound or a pharmaceutically acceptable salt thereof can be (S)-5-(4-(dimethylcarbamoyl)phenyl)-1-isopropyl-7-methyl-N-(4-(4-(vinylsulfonyl)piperazine-1-carboxamido)benzyl)-4,5,6,7-tetrahydro-1H-pyrazolo[4,3-c]pyridine-3-carboxamide.

A pharmaceutical composition can comprise a compound of Formula I.

The compositions of the disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). The compound can be administered by intravenous infusion or injection. The compound can be administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure. The oral administration may be in a powder or granule form. The oral dosage form can be sub-lingual, for example, a lozenge. In such solid dosage forms, the compounds of the disclosure are ordinarily combined with one or more adjuvants. Such capsules or tablets may comprise a controlled release formulation. In the case of capsules, tablets, and pills, the dosage forms also may comprise buffering agents or may be prepared with enteric coatings.

Oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

The disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

The disclosure comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of the disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of the disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosaqe Forms and Druq Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5)alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250, 500, or 750 milligrams (mg) of the active ingredient for the symptomatic adjustment of the dosage to the patient. The medicament can contain 0.01 mg to 1000 mg or 0.01 mg to 800 mg. A medicament typically contains 0.01 mg to 500 mg of the active ingredient, or 1 mg to 100 mg of active ingredient. Intravenous doses may be in the range of 0.01 to 10 mg/kg/minute during a constant rate infusion.

Liposome containing compounds of the disclosure may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the disclosure, which matrices are in the form of shaped articles, e.g., films, or microcapsules. Examples of sustained-release matrices include polyesters, hydrogels (for example, poly(2-hydroxyethyl-methacrylate) or poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the disclosure are generally placed into a container having a sterile access port, for example, an intravenous solution bag or vial having a stopper pierceable by a hypodermic injection needle.

Suitable emulsions may be prepared using commercially available fat emulsions, such as a lipid emulsion comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets having a diameter of 0.1 to 1.0 micrometers (μm), particularly 0.1 to 0.5 μm, and have a pH of 5.5 to 8.0.

For example, the emulsion compositions may be those prepared by mixing a compound of the disclosure with a lipid emulsion comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. The compositions can be administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent, or intermittent positive pressure breathing machine. Solution, suspension or powder compositions may be administered, preferably, orally or nasally, from devices which deliver the formulation in an appropriate manner.

A drug product intermediate (DPI) is a partly processed material that must undergo further processing steps before it becomes bulk drug product. Compounds of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. The drug product intermediate can contain a compound of the disclosure isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. Amorphous solid dispersions can comprise a compound of the disclosure and a polymer excipient. Other excipients as well as concentrations of said excipients and the compound of the disclosure are well known in the art and are described in standard textbooks. See, for example, “Amorphous Solid Dispersions Theory and Practice” by Navnit Shah et al.

This disclosure further comprises use of a compound of the disclosure for use as a medicament (such as a unit dosage tablet or unit dosage capsule). This disclosure comprises the use of a compound of the disclosure for the manufacture of a medicament (such as a unit dosage tablet or unit dosage capsule) to treat one or more of the conditions previously identified in the above sections discussing methods of treatment. This disclosure further comprises a compound of the disclosure, or a pharmaceutically acceptable salt thereof, for use as a medicament and a compound the disclosure, or a pharmaceutically acceptable salt therefore, for use in any method of treatment herein disclosed.

Typically, a compound of the disclosure is administered in an amount effective to treat a condition as described herein. The compounds of the disclosure may be administered as compound per se, or alternatively, as a pharmaceutically acceptable salt. For administration and dosing purposes, the compound per se or pharmaceutically acceptable salt thereof will simply be referred to as the compounds of the disclosure.

The compounds of the disclosure are administered by any suitable route in the form of a pharmaceutical composition adapted to such a route, and in a dose effective for the treatment intended. The compounds of the disclosure may be administered orally, rectally, vaginally, parenterally, topically, intranasally, or by inhalation.

The compounds of the disclosure may be administered orally. Oral administration may involve swallowing, so that the compound enters the gastrointestinal tract, or buccal or sublingual administration may be employed by which the compound enters the bloodstream directly from the mouth.

The compounds of the disclosure may also be administered parenterally, for example, directly into the bloodstream, into muscle, or into an internal organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle (including microneedle) injectors, needle-free injectors, and infusion techniques.

The compounds of the disclosure may be administered topically to the skin or mucosa, that is, dermally or transdermally. The compounds of the disclosure may also be administered intranasally or by inhalation. The compounds of the disclosure may be administered rectally or vaginally. The compounds of the disclosure may also be administered directly to the eye or ear.

The dosage regimen for the compounds of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. The total daily dose of a compound of the disclosure can be 0.01 to 100 mg/kg (i.e., mg compound of the disclosure per kg body weight) for the treatment of the indicated conditions discussed herein. The total daily dose of the compound of the disclosure can be 0.1 to 50 mg/kg, or 0.5 to 30 mg/kg. It is not uncommon that the administration of the compounds of the disclosure will be repeated a plurality of times in a day (typically no greater than 4 times). Multiple doses per day typically may be used to increase the total daily dose, if desired.

The compounds of the disclosure may inhibit the activity of STAT6 and may be useful in the treatment, prevention, suppression, and amelioration of disease(s) or diseases, disorders, and conditions mediated by STAT6.

The compounds of the disclosure may be used to treat or prevent at least one disease or condition relating to an inflammatory disorder. The compounds of the disclosure may be used to treat or prevent at least one disease or condition including atopic dermatitis, eosinophilic gastritis, atopic keratoconjunctivitis, allergy (including food allergy, latex allergy, pet allergy, mold allergy, insect allergy, pollen allergy, dust allergy, seasonal allergy, ragweed allergy, drug allergy, allergic rhinitis, allergic rhinitis, allergic fungal rhinosinusitis, allergic contact dermatitis, and allergic bronchopulmonary aspergillosis), alopecia (including alopecia areata), Alzheimer's disease, arteritis, asthma, atherosclerosis, autoimmune disorders (including lupus nephritis, autoimmune hepatitis, myasthenia gravis, Guillain-Barre syndrome, and Graves' disease) Bechet's disease, bullous pemphigoid, cancer, chronic obstructive pulmonary disease, chronic pruritis, chronic urticaria, celiac disease, Crohn's disease (CD), dermatitis (including atopic dermatitis (AD), hand dermatitis, and hand or foot atopic dermatitis), diabetic kidney disease, diversion colitis, eosinophilic esophagitis (including pediatric eosinophilic esophagitis), eye disorders or conditions (including autoimmune diseases of the eye, keratoconjunctivitis, vernal conjunctivitis, non-infectious uveitis (e.g. uveitis associated with Bechet's disease and lens-induced uveitis), keratitis (e.g. herpetic keratitis and conical keratitis), keratoleukoma, ocular premphigus, Mooren's ulcer, scleritis, retinitis, retinopathy, Grave's ophthalmopathy, Vogt-Koyanagi-Harada syndrome, keratoconjunctivitis sicca (dry eye), phlyctenule, iridocyclitis, endocrine ophthalmopathy, sympathetic ophthalmitis, allergic conjunctivitis, and ocular neovascularization), fungal keratitis, gout, idiopathic pulmonary fibrosis (IPF), indeterminate colitis, keloids, mastocytosis, microscopic colitis (such as collagenous colitis or lymphocytic colitis), myositis, nephritis, non-alcoholic steatohepatitis (NASH), pancreatitis, primary biliary cirrhosis, proctitis, prurigo nodularis, psoriasis, psoriatic arthritis, primary biliary cirrhosis, rhinosinusitis (including chronic rhinosinusitis with or without nasal polyps), sarcoidosis, scleroderma, sclerosing cholangitis, Sjogren's syndrome, systemic lupus erythematosus (SLE), systemic sclerosis, thyroiditis, ulcerative colitis (UC), vitiligo, vasculitis, Vogt-Koyanagi-Harada syndrome, Wegener's granulomatosis, or hidradenitis suppurativa. A method of treating or preventing one or one or more symptoms associated with the respective disease or condition comprises administering a pharmaceutical composition comprising a therapeutically effective amount of a compound of the disclosure to a patient.

The compounds of the disclosure may be used to treat or prevent at least one disease or condition including atopic dermatitis, asthma, chronic obstructive pulmonary disease (COPD), rhinosinusitis (for example, chronic rhinosinusitis with nasal polyps or chronic rhinosinusitis without nasal polyps), chronic urticaria (for example, chronic inducible urticaria or chronic spontaneous urticaria), prurigo nodularis, eosinophilic esophagitis, eosinophilic gastritis, bullous pemphigoid, pruritis (for example, chronic pruritis of unknown origin), atopic keratoconjunctivitis, chronic inducible urticaria, or allergy (for example, food allergy).

The compounds of the disclosure may be used to treat or prevent dermatological conditions, such as eczema (e.g. chronic and dyshidrotic eczema), chronic itch, dermatitis (e.g. atopic, irritant contact, allergic contact, occupational, perioral, stasis, nummular, seborrheic, xerotic, eyelid, diaper, and hand dermatitis), vitiligo, alopecia, alopecia areata, pruritis (e.g. chronic idiopathic pruritus), prurigo nodularis, psoriasis (e.g. plaque, guttate, inverse, pustular, nail, flexural palmoplantar, facial or erythrodermic psoriasis), scleroderma, pemphigus, dermatomyositis, neurodermatitis, skin flushing, cutaneous lupus erythematosus (e.g. acute cutaneous lupus (acute skin lupus), subacute cutaneous lupus (subacute lupus), and chronic cutaneous lupus (discoid lupus)), keloid, sunburn, hypertrophic scar, idiopathic thrombocytopenic purpura (ITP), ichthyosis (e.g. ichthyosis vulgaris), epidermal hyperplasia, acne, lichen planus, lichen sclerosis, rosacea, epidermolysis bullosa, intertrigo, keratosis pilaris, urticaria (e.g. chronic spontaneous urticaria, chronic idiopathic urticaria, chronic physical urticaria), molluscum contagiosum, Netherton syndrome, Sweet's syndrome, pityriasis alba, vulvovaginitis, Sutton's nevus/nevi, post inflammatory hypopigmentation, senile leukoderma, chemical/drug-induced leukoderma, palmoplantar pustulosis, pemphigoid, pemphigoid nodularis, bullous pemphigoid, and hidradenitis suppurativa.

The compounds of the disclosure may be used to treat or prevent respiratory conditions, such as rhinitis (e.g. allergic and perennial rhinitis), rhinorrhea, nasal congestion, nasal inflammation, asthma (e.g. chronic asthma, inveterate asthma, late asthma, bronchial asthma, allergic asthma, intrinsic asthma, extrinsic asthma, and dust asthma), chronic obstructive pulmonary disease (COPD), idiopathic pulmonary fibrosis (IPF), chronic and acute bronchoconstriction, chronic bronchitis, emphysema, allergic bronchopulmonary aspergillosis, chronic eosinophilic pneumonia, acute lung injury (ACI), adult respiratory distress syndrome (ARDS), pulmonary vascular disease (PVD), pulmonary arterial hypertension (PAH), bronchiectasis, sinusitis, rhinosinusitis, allergic fungal rhinosinusitis, chronic rhinosinusitis with nasal polyps, pulmonary sarcoidosis, and silicosis.

The compounds of the disclosure may be used to treat or prevent joint disorders, such as arthritis (e.g. osteoarthritis, as well as psoriatic, rheumatoid, juvenile, and gouty arthritis), spondyloarthropathy (e.g. reactive arthritis (also known as Reiter's Syndrome) and axial spondyloarthritis (including ankylosing spondylitis)), cartilage inflammation, bone degradation, and Still's disease; cardiovascular and metabolic disorders, such as diabetes (type 1 and type 2), myocarditis, diabetic neuropathy, atherosclerosis, cachexia, and celiac sprue; neuroinflammatory disorders, such as lupus (e.g. CNS, systemic and discoid lupus), systemic lupus erythematosus (SLE), diabetic neuropathy, autoimmune encephalitis, Alzheimer's disease, Parkinson's disease, and multiple sclerosis; and cancer.

The compound of the disclosure may be used alone, or in combination with one or more other therapeutic agents. The disclosure provides any of the uses, methods or compositions as defined herein wherein the compound of the disclosure, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compound at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the disclosure and the one or more other therapeutic agents may be administered as a fixed or non-fixed combination of the active ingredients. The term “fixed combination” means a compound of the disclosure, or a pharmaceutically acceptable salt thereof, and the one or more therapeutic agents, are both administered to a subject simultaneously in a single composition or dosage. The term “non-fixed combination” means that a compound of the disclosure, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

The compounds of the disclosure can be administered in combination with one or more pharmaceutically active agents other than the compounds of the disclosure including the pharmaceutically acceptable salts of the specifically named agents and the pharmaceutically acceptable solvates of said agents and salts.

The present disclosure can provide a pharmaceutical composition comprising the compound of the disclosure or a pharmaceutically acceptable salt thereof, wherein the pharmaceutical composition is administered in combination with a pharmaceutical composition comprising a different pharmaceutically active compound or a pharmaceutically acceptable salt thereof simultaneously or at different times.

These agents and compounds may be combined with pharmaceutically acceptable vehicles such as saline, Ringer's solution, dextrose solution, and the like. The particular dosage regimen, i.e., dose, timing and repetition, will depend on the particular individual and that individual's medical history.

Another aspect of the disclosure provides kits comprising the compound of the disclosure or pharmaceutical compositions comprising the compound. A kit may include, in addition to the compound of the disclosure or pharmaceutical composition thereof, diagnostic or therapeutic agents. A kit may also include instructions for use in a diagnostic or therapeutic method. The kit can include the compound or a pharmaceutical composition thereof and a diagnostic agent. The kit can include the compound or a pharmaceutical composition thereof and one or more therapeutic agents. The kits can be suitable for use in performing the methods of treatment described herein.

The kit can contain a first dosage form comprising one or more of the compounds of the disclosure in quantities sufficient to carry out the methods of the disclosure. The kit can comprise one or more compounds of the disclosure in quantities sufficient to carry out the methods of the disclosure and a container for the dosage and a container for the dosage.

Compounds of the present disclosure may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of Formula I. It will be further appreciated that it may be necessary or desirable to carry out the transformations in a different order from that described in the schemes, or to modify one or more of the transformations, to provide the desired compound of Formula I.

In the preparation of compounds of Formula I it is noted that some of the preparation methods useful for the preparation of the compounds described herein may require protection of remote functionality (e.g., a primary amine, secondary amine, carboxyl, etc. in a precursor of a compound of Formula I). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains amine or carboxylic acid functionality, such functionality may interfere with reactions at other sites of the molecule if left unprotected. Accordingly, such functionalities may be protected by an appropriate protecting group (PG) that may be removed in a subsequent step. Suitable protecting groups for amine and carboxylic acid protection include those protecting groups commonly used in peptide synthesis (such as N-t-butoxycarbonyl (BOC), benzyloxycarbonyl (Cbz), and 9-fluorenylmethylenoxycarbonyl (Fmoc) for amines and lower alkyl or benzyl esters for carboxylic acids) that are generally not chemically reactive under the reaction conditions described and may typically be removed without chemically altering other functionality in a compound of Formula I.

One skilled in the art will recognize that, in some cases, the compounds will be generated as a mixture of diastereomers and/or enantiomers; these may be separated at various stages of the synthetic scheme using conventional techniques or a combination of such techniques, such as, but not limited to, crystallization, normal-phase chromatography, reversed-phase chromatography and chiral chromatography, to afford the single enantiomers of the disclosure; for example, see “Stereochemistry of Organic Compounds” by E. L. Eliel and S. H. Wilen (Wiley, New York, 1994).

Unless otherwise indicated, the substituents in the schemes are defined as above. Isolation and purification of the products are accomplished by standard procedures, which are known to a chemist of ordinary skill.

It will be understood by one skilled in the art that the various symbols, superscripts, and subscripts used in the schemes, methods, and examples are used for the convenience of representation and/or to reflect the order in which they are introduced in the schemes and are not intended to necessarily correspond to the symbols, superscripts or subscripts in the appended claims. The schemes are representative of methods useful in synthesizing the compounds of the present disclosure. They are not to constrain the scope of the disclosure in any way.

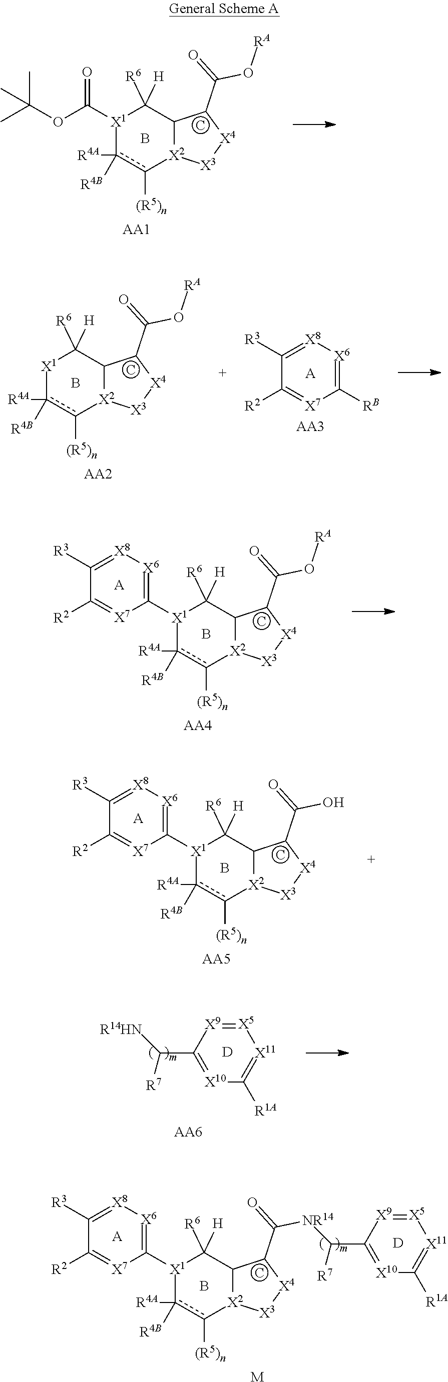

The general structure of Formula M can be prepared as shown in General Scheme A. The tert-butyloxycarbonyl (BOC) protected amine of Formula AA1, synthesized by literature methods or purchased commercially, can be treated with standard acidic deprotection reagents such as hydrochloric acid (HCl) in solvents like 1,4-dioxane (dioxane), ethyl acetate (EtOAc) and dichloromethane (DCM) from 10 degrees Celsius (°C) to room temperature to afford the deprotected amine compound of Formula AA2. The amine of Formula AA2 can be treated with a halogen (e.g., R^{B}═F or Br)-substituted aromatic of Formula AA3 using standard S_{N}Ar conditions such as N,N-diisopropylethylamine (DIEA), tripotassium phosphate (KPO₄), potassium carbonate (K₂CO₃), or cesium fluoride (CsF) in 1-pentanol, butyronitrile, N,N-dimethylformamide (DMF), dimethyl sulfoxide (DMSO), or acetonitrile (ACN) when heated between 80 to 140 °C to afford Formula AA4. The Formula AA4 can also be prepared by reacting Formula AA2 and AA3 with copper(I) iodide (CuI), 1,2-dimethylethylenediamine (DMEDA), or trans-N,N′-dimethylcyclohexane-1,2-diamine or Pd catalyst such as (2-dicyclohexylphosphino-2′,6′-diisopropoxy-1,1′-biphenyl)[2-(2′-amino-1,1′-biphenyl)]palladium(II) methanesulfonate (RuPhos Pd G3) with 2-dicyclohexylphosphino-2′,6′-diisopropoxybiphenyl (RuPhos), mesylate[(di(1-adamantyl)-N-butylphosphine)-2-(2′-amino-1,1′-biphenyl)]palladium(II) (cataCXium™ A Pd G3), [dicyclohexyl(2′,6′-diisopropoxy-2-biphenylyl)phosphine-κP](methanesulfonatato-κO)[2′-(methylamino-κN)-2-biphenylyl-κC²]palladium (RuPhos Pd G4) with a base such as sodium tert-butoxide (NaOtBu), K₂CO₃, K₃PO₄ or cesium carbonate (Cs₂CO₃) in a solvent like dioxane, dimethylacetamide (DMA), DMF, DMSO, 2-methyl-2-butanol (tert-amyl alcohol) or toluene and heated between 80 to 130 °C.

Formula AA4, where R³ is a halogen (e.g., F, Br, or I), can be converted to an amine, urea, or lactam using standard S_{N}Ar or cross coupling conditions described above to afford Formula AA4. When R³ is a halogen (e.g., I) on Formula AA4, the halogen can react with a BOC-protected hydrazine with the cross coupling conditions described above then can be deprotected under standard acidic conditions such as HCl in a solvent like dioxane or DCM and cyclized by reacting with (E)-4-(dimethylamino)but-3-en-2-one in EtOH to converted R³ to a 5-methyl-1H-pyrazole on Formula AA4. For Formula AA4, where R³ is a halogen (e.g., Br, I), can be converted to a carbon-carbon bond by reacting the halogen with nickel(II) chloride ethylene glycol dimethyl ether complex (NiCl₂·glyme), 5-methoxypicolinimidamide, and using activated ester of N-hydroxyphthalimide, tetrabutylammonium iodide, preactivated zinc (Zn) and trifluoroacetic acid (TFA) in a solvent like DMA from room temperature to 70 °C or under photoredox conditions such as [4,4′-bis(1,1-dimethylethyl)-2,2′-bipyridine-N1,N1′]bis[2-(2-pyridinyl-N)phenyl-C]iridium(III) hexafluorophosphate (Ir(ppy)₂(dtbbpy)PF₆), nickel(II) bromide ethylene glycol dimethyl ether complex (NiBr₂·DME), 5-methoxypicolinimidamide, phthalimide and quinuclidine in a solvent such as DMA and methyl tertiary-butyl ether (MTBE). When R³ is a halogen (e.g., —Br) on Formula AA4, the halogen can react with bis(pinacolato)diboron (B₂Pin₂) and (1,1′-bis(diphenylphosphino)ferrocene)palladium(II) dichloride (Pd(dppf)Cl₂) with a base like potassium acetate (KOAc) in a solvent like dioxane at 85 °C to form boronic acid at R³. The boronic acid at R³ can react further by reacting with bromo-substituted 5-membered ring heterocycle, Pd(dppf)Cl₂ and with a base like K₃PO₄ in solvents like dioxane at 85 °C to give a 5-membered ring heterocycle at R³ on Formula AA4. When R³ is a carboxylic acid on Formula AA4, the acid can be transformed further via amide coupling with HATU and a base like DIEA in a solvent like DMF at 40 °C to form the amide at R³ on Formula AA4. Formula AA4, when R³ is a tert-butyl acetate, can be deprotected under standard acidic conditions such as TFA or HCl in a solvent like dioxane, H₂O, and DCM from room temperature to 40 °C, which can be transformed further under standard amide coupling conditions to form an amide at R³ on Formula AA4. Formula AA4, when R³ is a methyl propionate, can react with 2-bromoacetonitrile and a base like lithium bis(trimethylsilyl)amide (LiHMDS) in a solvent like THF at −78 °C, which can be transformed further by reacting with sodium borohydride (NaBH₄) in solvents like THF and H₂O at 0 °C to room temperature to form a 5-membered lactam at R³ on Formula AA4.

The ester (e.g., R^{A}=methyl or ethyl) of Formula AA4 can undergo standard hydrolysis conditions such as utilizing a base (e.g., lithium hydroxide monohydrate (LiOH), sodium hydroxide (NaOH), 1,5,7-triazabicyclo[4.4.0]dec-5-ene, or potassium trimethylsilanolate (KOTMS) in solvents like tetrahydrofuran (THF), methanol (MeOH), ethanol (EtOH), water (H₂O), dioxane, or ACN. The reaction can typically occur at 0 to 50 °C, preferably at about 23 °C (room temperature), to afford the carboxylic acid of Formula AA5.

The compound of Formula M can be obtained by reacting the carboxylic acid of AA5 with the amine of Formula AA6 under standard amide coupling reagents like 1-(3-dimethylaminopropyl)-3-ethylcarbodiimide hydrochloride (EDCI) with 2-hydroxypyridine-N-oxide (HOPO), 1-[bis(dimethylamino)methylene]-1H-1,2,3-triazolo[4,5-b]pyridinium 3-oxid hexafluorophosphate (HATU), or other common reagents in solvents such as ACN and H₂O, DMF, or DMSO along with an organic base such as triethylamine (TEA) or DIEA stirred at a temperature of 0 to 70 °C, preferably at about 23 °C (room temperature).

Formula M, when R¹ (when X¹¹ is CR¹) is an amine, can react with 2,2,2-trichloroethyl carbonochloridate in pyridine from 0 °C to room temperature, which can transform further with a secondary amine and 1,8-diazabicyclo[5.4.0]undec-7-ene (DBU) in THF from 40-60 °C to form a urea at R¹. Formula M, when R¹ is phenyl carbamate, can react with an amine and a base like TEA in a solvent like DMF from 40 °C, which can then undergo a deprotection under acidic conditions such as HCl in a solvent like dioxane and DCM from 0 °C to room temperature to form a urea at R³. Formula M, when R³ is a tert-butyl acetate, can be deprotected under standard acidic conditions, such as HCl in a solvent like H₂O and DCM to form a carboxylic acid at R³ on Formula M. The carboxylic acid at R³ of Formula M can then undergo an amide coupling with a primary to secondary amine by reacting with HATU and DIEA in DMF at 35 °C, which can react further via an acidic deprotection with TFA in DCM at 30 °C to remove a BOC-protecting group. Formula M (when X¹¹ is CR¹) when R¹ is an amine, can react with ethyl carbonochloridate with a base like TEA in a solvent like DCM from 0 °C to room temperature to form an amide at R¹ on Formula M. Formula M can undergo separation through chiral separation methods, such as supercritical fluid chromatography (SFC), to provide the single diastereomers of the target compound.

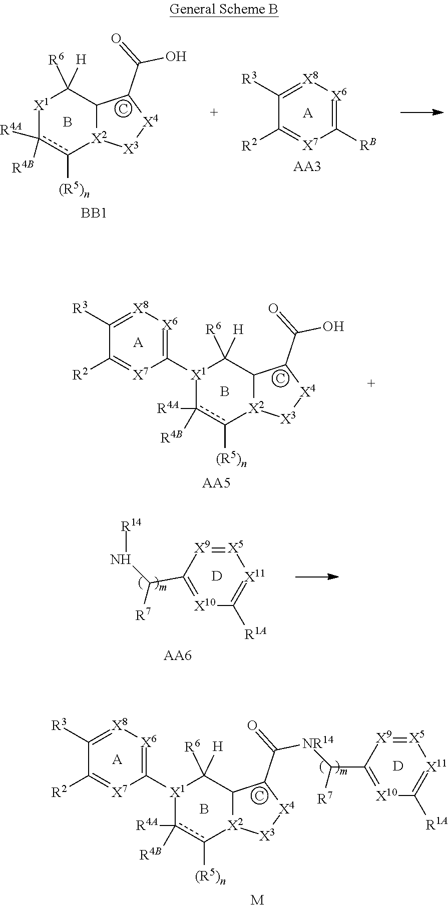

The general structure of Formula M can be prepared as shown in General Scheme B. The amine of Formula BB1 can be treated with a halogen (e.g., R^{B}═F or Cl)-substituted aromatic of Formula AA3 using standard SNAr conditions such as cesium fluoride (CsF) or K₃PO₄ in a solvent like water or DMSO and stirred at 80 to 140 °C to afford Formula AA5. An alternate method to form Formula AA5 is through a palladium coupling reaction by reacting Formula BB1 and Formula AA3 with a catalyst like RuPhos Pd G3 or chloro[(tri-tert-butylphosphine)-2-(2-aminobiphenyl)]palladium(II) (P(t-Bu)₃ Pd G2) with a base like K₃PO₄ or KOTMS in a solvents like DMSO, DMF or tert-amyl alcohol from 95 to 110 °C. The compound of Formula M can be obtained by reacting the carboxylic acid of Formula AA5 with the amine of Formula AA6 under standard amide coupling reagents like EDCI with HOPO, HATU, or other common amide coupling reagents in solvents such as ACN and H₂O, DMF, or DMSO along with an organic base such as TEA or DIEA stirred at a temperature of 0 to 70 °C, preferably at room temperature.

Formula M, when R⁶ is a hydrogen, can be oxidized with 2,3-dichloro-5,6-dicyano-p-benzoquinone (DDQ) in acetone and then further transformed to methylate when reacted with boron trifluoride diethyl etherate (BF₃OEt₂) and dimethylzinc in solvents like toluene and THF. Formula M, when R³ is a tert-butyl acetate, can be deprotected under standard acidic conditions, such as HCl in a solvent like dioxane, H₂O, and DCM, which can be transformed further under standard amide coupling conditions to form an amide at R³. Formula M, when R¹ is a bromine, can undergo a Buchwald coupling with [(2-di-tert-butylphosphino-3,6-dimethoxy-2′,4′,6′-triisopropyl-1,1′-biphenyl)-2-(2′-amino-1,1′-biphenyl)]palladium(II) (tBuBrettPhos Pd G3), an amine bonded to a heterocycle and a base like Cs₂CO₃ in dioxane at 90 °C to provide Formula M with R¹ as an amine bonded to heterocycles.

Formula M can undergo separation through chiral separation methods, such as supercritical fluid chromatography (SFC), to provide the single diastereomers of the target compound.

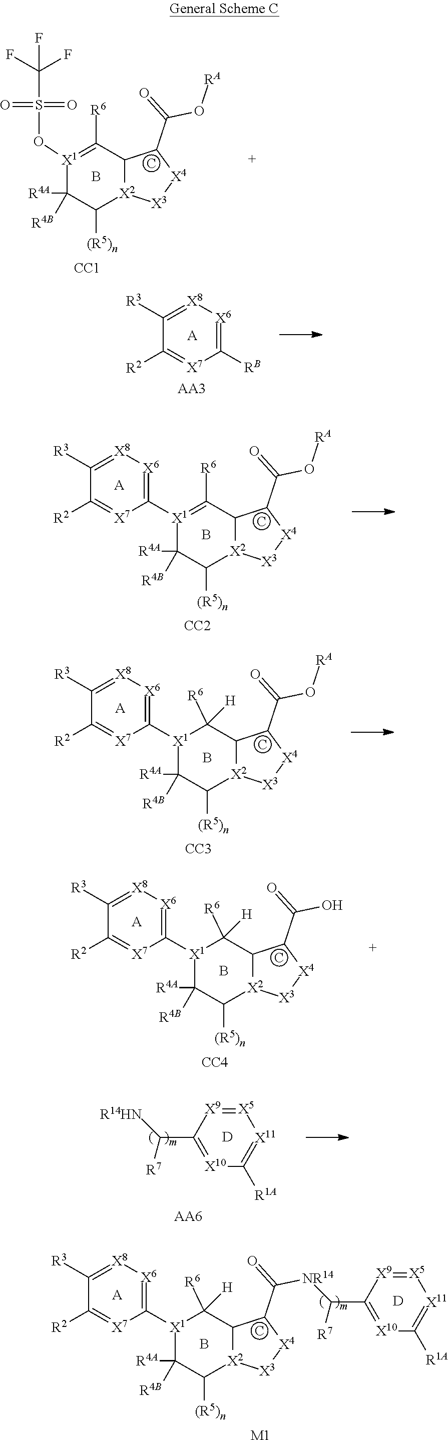

The general structure of Formula M1 can be prepared as shown in General Scheme C. The trifluoromethanesulfonic acid of Formula CC1, synthesized by literature methods or purchased commercially, can be treated with a boronic acid (e.g., R^{B}═B—(OH)₂)-substituted aromatic of Formula AA3 using with a Pd catalyst such as Pd(dppf)Cl₂ and a base like K₃PO₄ in solvents such as H₂O and THF at 85 °C to form Formula CC2.

The trifluoromethanesulfonic acid of Formula CC1 can also be treated with B₂Pin₂ with a base like KOAc and a palladium catalyst such as Pd(dppf)Cl₂ in dioxane at 80 °C to form the boronate ester in Formula CC1, which can further react with a base like K₂CO₃ and Pd(dppf)Cl₂ in dioxane and H₂O at 80 °C to form Formula CC2.

The alkene of Formula CC2 can undergo hydrogenation with palladium on carbon (Pd/C) in EtOH or Pd/C in EtOH with ammonium formate (NH₄HCO₂) to reduce the alkene to the alkane bond in Formula CC3. The ester (e.g., R^{A}=ethyl) of Formula CC3 can undergo standard hydrolysis conditions by utilizing a base like LiOH in solvents like THF, MeOH, and H₂O from 0 to 50 °C, preferably at room temperature, to afford the carboxylic acid of Formula CC4. The compound of Formula M1 can be obtained by reacting with the carboxylic acid of Formula CC4 and the amine of Formula AA6 under standard amide coupling reagents like EDCI with HOPO in solvents such as DMSO along with an organic base such as DIEA stirred at 0 to 70 °C.

Formula M1 can undergo separation through chiral separation methods, such as supercritical fluid chromatography (SFC), to provide the single diastereomers of the target compound.

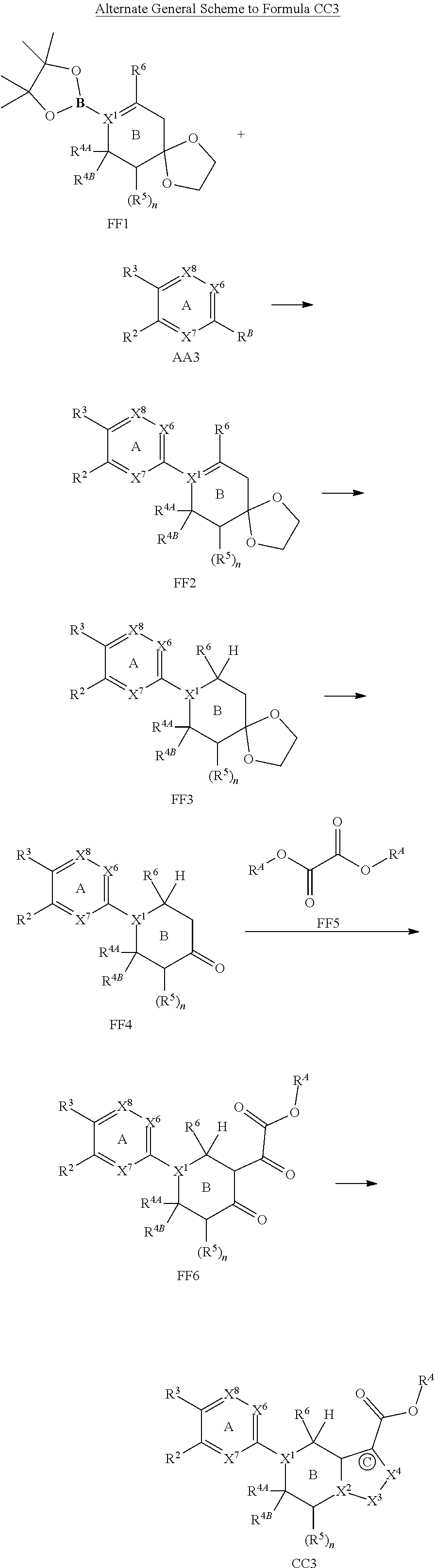

The boronic ester of Formula FF1, synthesized by literature methods or purchased commercially, can be treated with Formula AA3 when R^{B} is a chloride by using a Pd catalyst such as Pd(dppf)Cl₂ and a base like K₂CO₃ in solvents such as dioxane at 80 °C to form Formula FF2. The alkene of Formula FF2 can reduce under standard hydrogenation conditions such as Pd/C in solvents like THF and EtOAc under hydrogen gas at 50 psi to give the alkane bond in Formula FF3. The dioxolane ring of Formula FF3 can undergo an acid-catalyzed hydrolysis with HCl in H₂O and acetone to give the carbonyl of Formula FF4. The carbonyl of Formula FF4 can react with the alkyl oxalate of Formula FF5 under basic conditions such as LiHMDS in THF at −78 °C to room temperature to give the oxoacetate of Formula FF6, which can further react with hydrazine dihydrochloride and a base such as K₃PO₄ in EtOH to give the cyclized Formula CC3. Formula CC3 can be further functionalized as shown in General Scheme C to prepare Formula II.

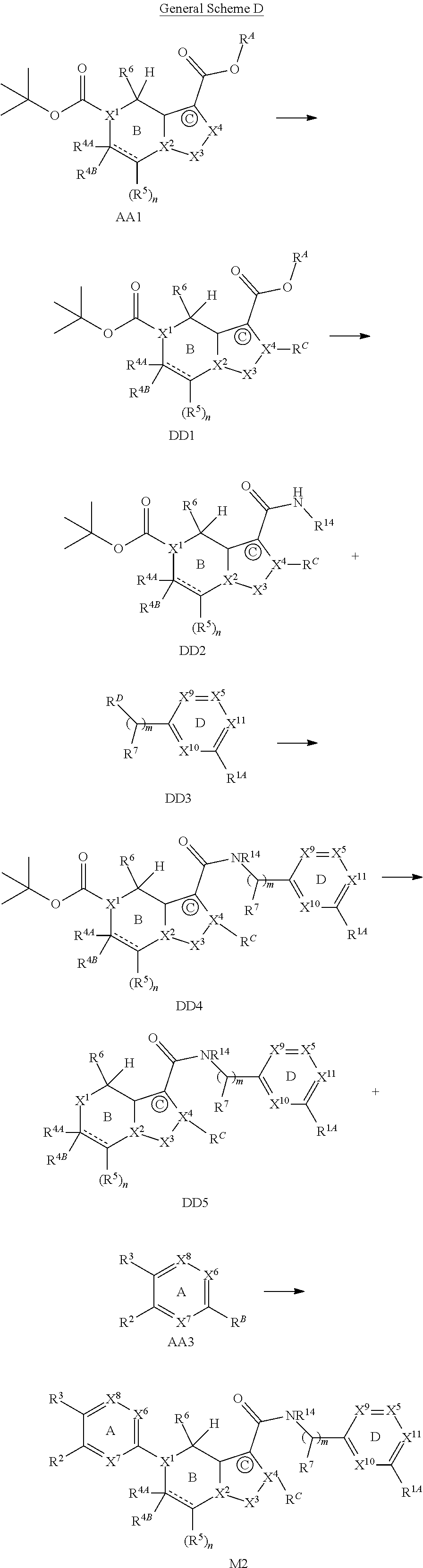

The general structure of Formula M2 can be prepared as shown in General Scheme 0. The amine of Formula AA1, synthesized by literature methods or purchased commercially, can react with a bromo substituted alkyl amine with a benzyloxy carbonyl (Cbz) protecting group and a base like Cs₂CO₃ in ACN to give mixture of N1 and N2-alkylated (e.g., R^{C}=Cbz protected alkyl amine) compounds of Formula DD1. The regio-isomers can be separated by silica gel column purification to provide compounds of Formula DD1. Formula DD1, when R^{c} is a Cbz protected alkyl amine, can then undergo deprotection of the CBz protecting group by reacting with Pd/C in a solvent like EtOH at 50-120 pounds per square inch (psi) of hydrogen (H₂) gas to give Formula DD1 when R^{c} is an alkyl amine. The free amine on the N2 of Formula DD1 can react with DBU in a solvent like dioxane at 100 °C to cyclize to form a C₆₋₇ heterocycle fused to Ring C of Formula DD2. The cyclized lactam of Formula DD2 can be treated with a halogen (e.g., R^{D}═Br)-substituted aromatic of Formula DD3 with bases such as K₂CO₃, potassium bis(trimethylsilyl)amide (KHMDS) in THF with additional solvents such as ACN and DMF with stirring at 80 °C to afford Formula DD4. The BOO protected amine of Formula DD4 can be deprotected under acidic conditions such as HCl in solvents like dioxane and DCM to form the amine of Formula DD5. The amine of Formula DD5 can be treated with a halogen (e.g., R^{B}═F)-substituted aromatic of Formula AA3 using standard SNAr conditions such as K₃PO₄ in a solvent like tert-amyl alcohol and H₂O with stirring at 100 °C to afford Formula M2. Formula M2, when R¹ is an iodine, can react further with a urea, a base like Cs₂CO₃ and palladium catalyst such as ((SP-4-3)-[[5-(diphenylphosphino)-9,9-dimethyl-9H-xanthen-4-yl]diphenylphosphine-κP](methanesulfonato-κO)[2′-(methylamino-κN)[1,1′-biphenyl]-2-yl-κC]-palladium) XantPhos Pd G4 in a solvent like tert-amyl alcohol at 90 °C to form Formula M2 with R¹ as an urea.

Formula M2 can undergo separation through chiral separation methods, such as supercritical fluid chromatography (SFC), to provide the single diastereomers of the target compound.

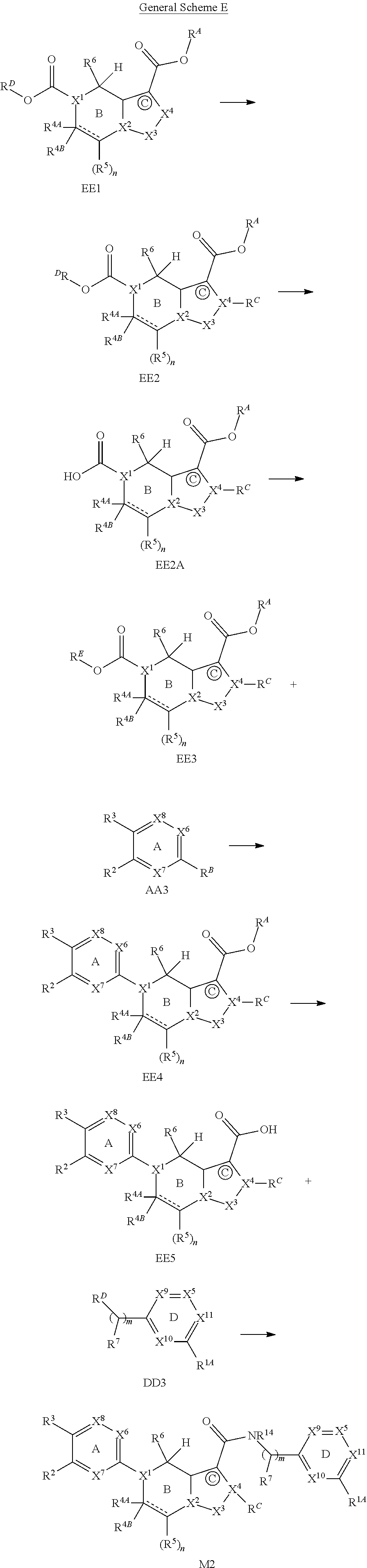

The general structure of Formula II can be prepared as shown in General Scheme E. The ester of Formula EE1 (e.g., R^{D}=tBu) synthesized by literature methods or purchased commercially. The amine of Formula EE2, can react with a bromo or iodo substituted alkyl group with a base like Cs₂CO₃ or LiHMDS in solvents such as DMF, THF or ACN at 0 °C to room temperature to give mixture of N1 and N2-alkylated compounds of Formula EE2. The regioisomers can be separated by silica gel column purification to provide compounds of Formula EE2. The ester (e.g., R^{D}=tBu), can undergo an acid-catalyzed hydrolysis with an acid such as HCl or TFA in solvents like THF, MeOH, EtOH, H₂O, dioxane, or ACN which can typically occur between 0 to 50 °C, preferably at about 23 °C (room temperature), to afford the carboxylic acid of Formula EE2A. The carboxylic acid of Formula EE2A can undergo standard coupling reaction to form the activated esters of Formula EE3 (e.g., N-hydroxyphthamimide) using reagents like EDCI with HOPO, HATU or other common reagents in solvents such as ACN and H₂O, DMF, or DMSO along with an organic base such as TEA or DIEA stirred at a temperature of 0 to 70 °C, preferably at about 23 °C (room temperature). The C(sp3) radical precursors of formula EE3 can undergo decarboxylative C(sp2)-C(sp3) cross coupling with aryl halides of formula AA3 using nickel(II) chloride complexed with bidentate pyridine-based ligands (e.g., 2,2′-bipyridyl, picolinimidamide), a zinc reductant, and tetrabutylammonium iodide in solvents like DMF, DMA, orACN. The reaction typically occurs at 0 to 100 °C, preferably at about 23 °C (room temperature), to afford the C(sp2)-C(sp3) coupled product of Formula EE4. The ester (e.g., R^{A}=methyl or ethyl) of Formula EE4 can undergo an base-catalyzed hydrolysis by utilizing a base such as LiOH, NaOH, TBD, or KOTMS in solvents like THF, MeOH, EtOH, H₂O, dioxane, or ACN. The reaction can typically occur at 0 to 50 °C, preferably at about 23 °C (room temperature), to afford the carboxylic acid of Formula EE5. The compound of Formula M2 can be obtained by reacting the carboxylic acid of Formula EE5 with the amine of Formula AA6 under standard amide coupling reagents like EDCI with HOPO, HATU, or other common reagents in solvents such as ACN and H₂O, DMF, or DMSO along with an organic base such as TEA or DIEA stirred at a temperature of 0 to 70 °C, preferably at about 23 °C (room temperature).

Formula M2 can undergo separation through chiral separation methods, such as supercritical fluid chromatography (SFC), to provide the single diastereomers of the target compound.

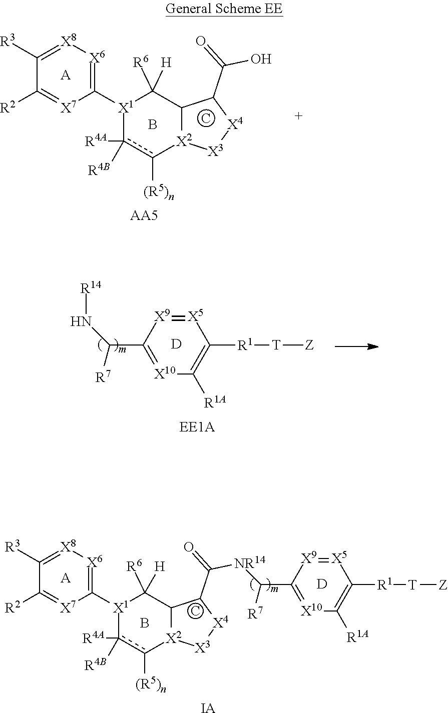

The general structure of Formula IA can be prepared as shown in General Scheme EE. The compound of Formula IA can be obtained by reacting the carboxylic acid of Formula AA5 with the amine of Formula EE1A under standard amide coupling reagents like HATU or other common amide coupling reagents in solvents such as ACN and H₂O, DMF, or DMSO along with an organic base such as TEA or DIEA stirred at a temperature of 0 to 70 °C, preferably at room temperature.

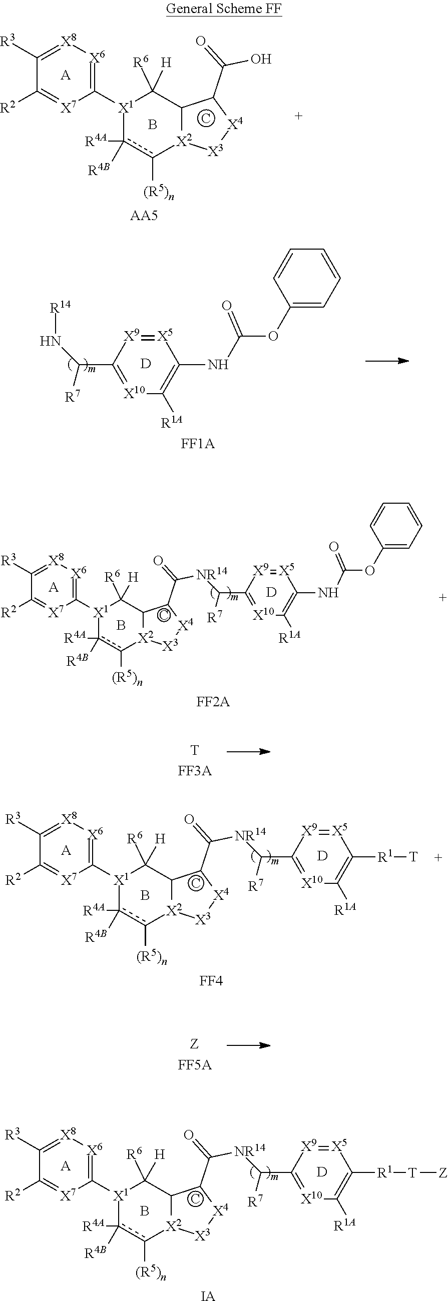

The general structure of Formula IA can be prepared as shown in General Scheme FF. The compound of Formula FF2A can be obtained by reacting the carboxylic acid of AA5 with the amine of Formula FF1A under standard amide coupling reagents like HATU or other common amide coupling reagents in solvents such as DMF or DMSO along with an organic base such as TEA or DIEA stirred at a temperature of −5 to 70 °C, preferably at room temperature. Formula FF2A can react with Formula FF3A, where Formula FF3A has a reactive amine, under basic conditions such as TEA in a solution of DMF at 40 °C to form Formula FF4A. If Formula FF4A contains a BOC-protecting group on the linker, then a deprotection reaction can occur under acidic conditions such as HCl in dioxane from 0 °C to room temperature to give an amine of Formula FF4A. The amine of Formula FF4A can react with the aldehyde of Formula FF5A under reductive amination conditions such as sodium cyanoborohydride (NaBH₃CN) or sodium triacetoxyborohydride (NaBH(OAc)₃) as the reducing agent with AcOK and acetic acid (AcOH) in solvents like THF and DMSO to form Formula IA.

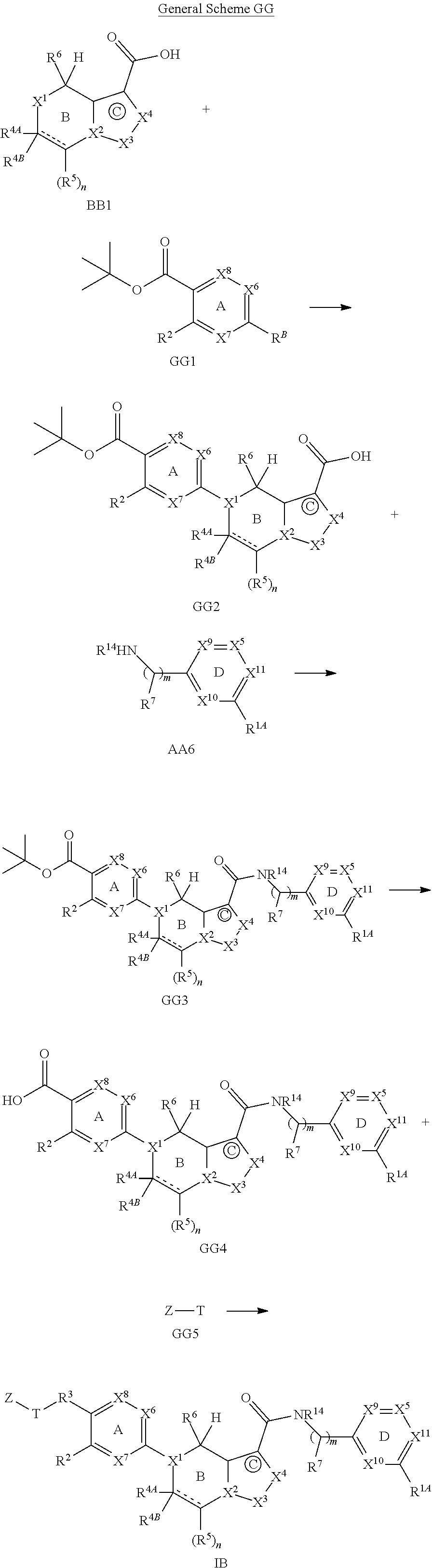

The general structure of Formula IB can be prepared as shown in General Scheme GG. The amine of Formula BB1 can be treated with a halogen (e.g., R^{B}═C₁)-substituted aromatic of Formula GG1 using palladium coupling conditions such as RuPhos Pd G3 with K₃PO₄ in solvents like DMF or DMSO and stirred at 80 to 140 °C to afford Formula GG2. The amide of Formula GG3 can be obtained by reacting the carboxylic acid of Formula GG2 with the amine of Formula AA6 under standard amide coupling reagents like EDCI with HOPO, HATU, or other common amide coupling reagents in solvents such as ACN and H₂O, DMF, or DMSO along with an organic base such as TEA or DIEA stirred at a temperature of 0 to 70 °C, preferably at room temperature. The tert-butyl ester of Formula GG3 can be deprotected under standard acidic conditions, such as HCl in a solvent like dioxane, H₂O, and DCM to form the acid of Formula GG4. The acid of Formula GG4 can react with Formula GG5, when T contains an amine, under standard amide coupling conditions like HATU, or other common amide coupling reagents in solvents such as DMF or DMSO along with an organic base such as TEA or DIEA stirred at a temperature of 0 to 70 °C to form Formula IB.

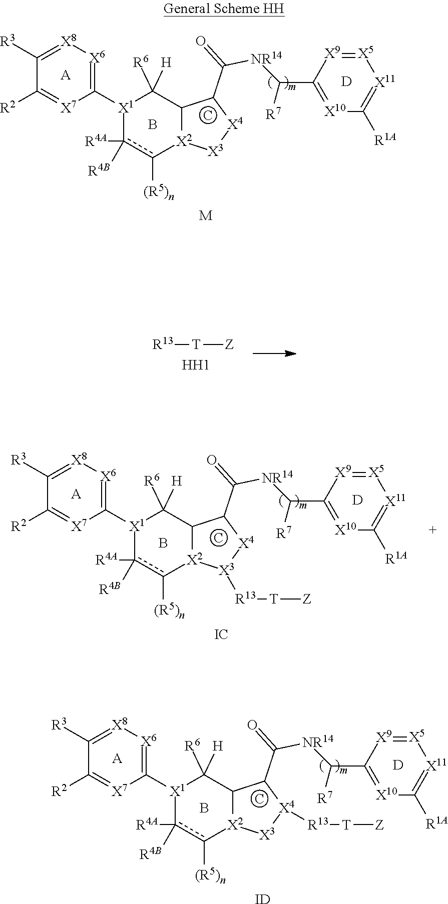

The general structure of Formula IC and Formula ID can be prepared as shown in General Scheme HH. Formula M can react with Formula HH1 under standard alkylation conditions, such as with a base like LiHMDS, Cs₂CO₃ or K₂CO₃ in a solvents like DMF or ACN at 0 to 50 °C to give mixture of N1 and N2-alkylated compounds of Formula IC and Formula ID.

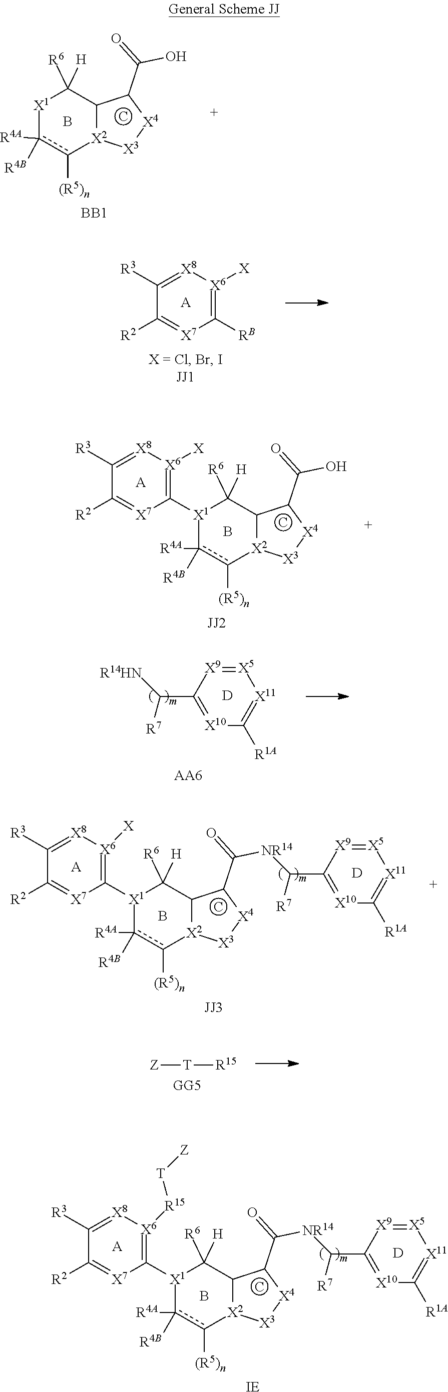

The general structure of Formula IE can be prepared as shown in General Scheme JJ. The amine of Formula BB1 can be treated with a halogen (e.g., R^{B}═F, Cl)-substituted aromatic of Formula JJ1 using palladium coupling conditions such as RuPhos Pd G3 with K₃PO₄ in solvents like DMF or DMSO and stirred at 80 to 140 °C or nucleophilic substitution reaction conditions such as CsF in DMSO or K₃PO₄ in water at 80-100 °C to afford Formula JJ2. The amide of Formula JJ3 can be obtained by reacting the carboxylic acid of Formula JJ2 with the amine of Formula AA6 under standard amide coupling reagents like EDCI with HOPO, HATU, or other common amide coupling reagents in solvents such as ACN and H₂O, DMF, or DMSO along with an organic base such as TEA or DIEA stirred at a temperature of 0 to 70 °C, preferably at room temperature. The compound of Formula IE can be obtained by reacting Formula JJ3, when X is Cl, Br and I, with Formula GG5, when R¹⁵-T-Z contains an amine or alcohol, with a base like Cs₂CO₃, DIEA or TEA in a solvent like DMF or ACN.

The compound of Formula IE can also be obtained by reacting Formula JJ3, when X is halogen, with Formula GG5, when R¹⁵-T-Z contains an aromatic ring substituted with one of the groups such as boronic ester, amine, phenol, alkyne or alkene, with a Pd catalyst such as Pd(dppf)Cl₂ and a base like K₃PO₄ in solvents such as H₂O and THF at 80 to 110 °C.

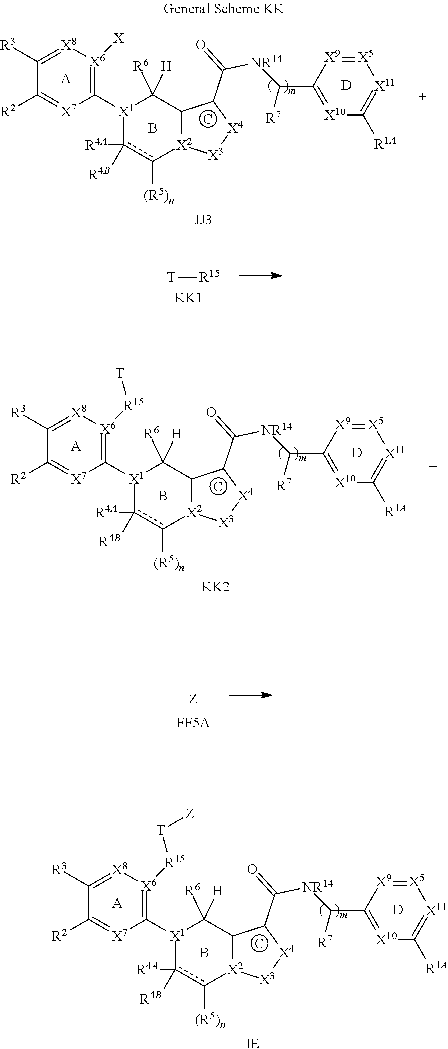

The general structure of Formula IE can be prepared as shown in General Scheme KK. The Formula JJ3, when X is halogen, can react with Formula KK1, when R¹⁵-T contains an aromatic substituted boronic ester with a Pd catalyst such as Pd(dppf)Cl₂ and a base like K₃PO₄ in solvents such as H₂O and THF at 80 to 110 °C to form Formula KK2. Formula KK2 can undergo an acid deprotection, with acids such as HCl or TFA in solvents like dioxane, when the R¹⁵-T contains a BOC-protecting group. The amine of Formula KK2 can react with Formula FF5A, when Z contains an aldehyde, under reductive amination conditions such as sodium triacetoxyborohydride (NaBH(OAc)₃) with AcOK and AcOH in solvents like THF and DMSO to form Formula IE.

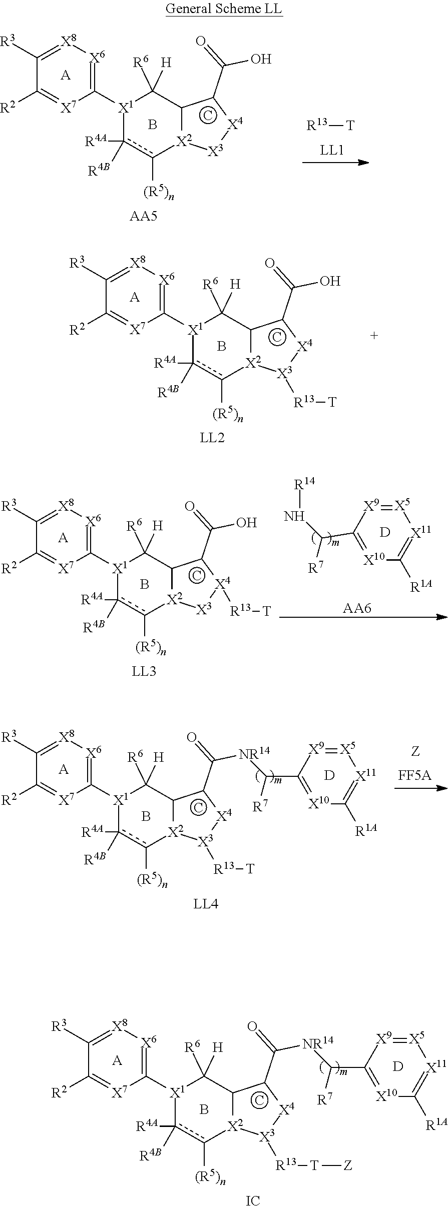

The general structure of Formula IC can be prepared as shown in General Scheme LL. Formula AA5 can react with Formula LL1, when R¹³ contains an alkyl halide, reaction under standard alkylation conditions, with a base such as LiHMDS, Cs₂CO₃ or K₂CO₃ in solvents like THF and DMF at 0 to 50 °C can give mixture of N1 and N2-alkylated compounds of Formula LL2 and LL3 which can be separated by column chromatography.

The acid of Formula LL2 can react with the amine of Formula AA6 under standard amide coupling reagents like EDCI with HOPO, HATU, or other common reagents in solvents such as ACN and H₂O, DMF, or DMSO along with an organic base such as TEA or DIEA stirred at a temperature of 0 to 70 °C, preferably at about 23 °C (room temperature) to give Formula LL4. When T contains an alkyne, Formula LL4 can react with the Formula FF5A, when Z contains a bromide, with CuI, bis(triphenylphosphine)palladium(II) dichloride (PdCl₂(PPh₃)₂) with a base like DIEA in a solvent like DMSO at 90-110 °C to form Formula IC.

In order that this disclosure may be better understood, the following examples are set forth. These examples are for the purpose of illustration only and are not to be construed as limiting the scope of the disclosure in any manner. Any of the substituents as defined herein and above or as illustrated in the examples, can be combined with any of the other substituents as defined herein and above or as illustrated in the examples to form the compounds of Formula M, M-1, M1, M2, IA, IB, IC, ID, and IE

### EXAMPLES

The compounds and intermediates described below were named using the naming convention provided with ChemDraw version 20.1.1.123 or Revvity ChemDraw version 25.5.0×64 or generated with ACD Labs software version “ACD/Name 2023.2.0”. The naming convention provided with ChemDraw version 20.1.1.123 and ACD/Name 2023.2.0 is well known by those skilled in the art and it is believed that the naming convention generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

The following illustrates the synthesis of various compounds of the present invention. Additional compounds within the scope of this invention may be prepared using the methods illustrated in these Examples, either alone or in combination with techniques generally known in the art.

All starting materials in these Preparations and Examples are either commercially available or can be prepared by methods known in the art or as described herein.

Commercial solvents and reagents were generally used without further purification. Anhydrous solvents were employed where appropriate, generally ACROSEAL™ products from Acros Organics, Aldrich SURE/SEAL™ from Sigma- Aldrich, or DRISOLV™ products from EMD Chemicals. Commercial solvents and reagents were used without further purification.

Experiments were generally carried out under inert atmosphere (nitrogen or argon), particularly in cases where oxygen- or moisture-sensitive reagents or intermediates were employed.

Unless otherwise noted, chemical reactions were performed at room temperature (about 23 degrees Celsius).

The terms “concentrated,” “evaporated,” “distillation to dryness,” “concentrated by reduced pressure,” and “concentrated in vacuo” refer to the removal of solvent at reduced pressure on a rotary evaporator with a bath temperature less than 60 degrees Celsius (°C).

In some examples, chiral separations were carried out to separate enantiomers or diastereomers of certain compounds of the disclosure.

For syntheses referencing procedures in other Examples or Methods, reaction conditions (reaction time and temperature) may vary. In general, reactions were followed by thin-layer chromatography (TLC) or mass spectrometry (MS) and subjected to work-up when appropriate.

Commercial benchtop photoreactors such as the Penn OC Photoreactor M2, Acceled Photoreactor M2 and Lumidox II with 24-well block were utilized with 450 or 445 nanometer (nm) light-emitting diode (LED).

Purifications may vary between experiments: in general, solvents and the solvent ratios used for eluents/gradients were chosen to provide appropriate retention times.

Reaction progress was monitored using thin layer chromatography (TLC), liquid chromatography-mass spectrometry (LCMS) and high-performance liquid chromatography (HPLC), analyses. TLC was performed on pre-coated silica gel plates with a fluorescence indicator (254 nm excitation wavelength) and visualized under ultraviolet (UV) light.

LCMS data were acquired on an Agilent 1100 Series instrument with a Leap Technologies autosampler, Gemini C₁₈ columns, ACN/water gradients, and either trifluoroacetic acid (TFA), formic acid, or ammonium hydroxide (NH₄OH) modifiers or similar equipment. The column eluent was analyzed using Waters ZQ mass spectrometer scanning in both positive and negative ion modes from 100 to 1200 Da. Other similar instruments were also used.

High resolution mass spectrometry (HR/MS) was gathered on Sciex TripleTOF 5600+ with DuoSpray ionization source with the liquid chromatography instrument with an Agilent Technologies, 1200 binary pump, Agilent 1200 autosampler, Agilent 1200 column compartment, and Agilent 1200 diode array detector. The instrument acquisition and data handling were done with Sciex Analyst TF version 1.7.1.

HPLC data were acquired on an Agilent 1100 Series instrument using Gemini, Sunfire, Welch Boltimate™, Welch Xtimate, Prep PG-45 MOD10, Chiral Tech ID, Boston Prime, Boston Green ODS, Phenomenex Gemini NX, XBridge C₁₈ columns or WePure Biotech XP tC₁₈ column with acetonitrile (ACN)/water (H₂O) gradients, and either TFA, formic acid, NH₄OH, or ammonium bicarbonate (NH₄HCO₃) modifiers and comparable equipment. Purifications were performed by high performance liquid chromatography (HPLC) and medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges and the like. Chiral purifications were performed by chiral supercritical fluid chromatography (SFC) using Berger or Thar instruments and similar instruments with DAICEL CHIRALCEL OD, OJ; DAICEL CHIRALPAKAD, AS, IF, ID; Chiral Technologies OJ-H, AD-H, OD-H, IA, IB; Lux Cellulose 1; Lux Cellulose 3; Pirkle Covalent (R,R) Whelk-01; CHIRALPAK IH; YMC-IB; Chiral Tech ID; and Phenomenex Lux Cellulose 1 columns; and carbon dioxide (CO₂) mixtures with methanol (MeOH), ethanol (EtOH), isopropyl alcohol, orACN, alone or modified using TFA, formic acid, NH₄OH, diethylamine (DEA), ammonia (NH₃) or isopropylamine. UV detection was used to trigger fraction collection.

The chiral purity was determined using supercritical fluid chromatography (SFC) and reverse-phase liquid chromatography (RPLC) analytical method with Chiralpak AD-3, IG-3, IF, AS-3, IB-N, ID; Chiralcel OJ-, OD-3; Chiral Technologies OJ-H, AD-H, OD-H, IA, IB, IH; Lux Cellulose 1; Chiral Tech ID; IM-3; Lux Cellulose 3; Phenomenex Kinetex and Regis (R,R) Whelk-01 columns; and CO₂ mixtures with MeOH, EtOH, isopropyl alcohol, or ACN, alone or modified using TFA, formic acid, NH₄OH, DEA, NH₃, or isopropylamine.

Proton nuclear magnetic resonance (¹H NMR) spectra were recorded, referenced to residual peaks from the deuterated solvents employed, where 6 is chemical shift; d is doublet; dd is doublet of doublets; m is multiplet, s is singlet, t is triplet, q, quartet; quin, quintet, br s, broad singlet; MHz is megahertz, ppm is parts per million. Proton nuclear magnetic spectroscopy (¹H NMR) chemical shifts are expressed in parts per million (ppm, d) referenced to the deuterated solvent residual peaks (CDCl₃, 7.26 ppm; (CD₃)₂SO, 3.33 or 2.50 ppm; CD₃OD is 4.87 or 3.31 ppm) were recorded on 300, 400, 500, or 600 MHz (where MHz is megahertz) Varian, Bruker, or Jeol spectrometers.

Products were generally dried under vacuum before being carried on to further reactions or submitted for biological testing.

In the experimental sections that follow the following abbreviations may be used. ACN is acetonitrile; abs in the structures is absolute and refers to the stereochemistry being confirmed as R or S; Å is angstrom; AcOH is acetic acid; APhos Pd G3 is [4-(di-tert-butylphosphino)-N,N-dimethylaniline-2-(2′-aminobiphenyl)]palladium(II) methanesulfonate; BBr₃ is boron tribromide; B₂Pin₂ is bis(pinacolato)diboron; BOC is tert-butyloxycarbonyl; Boc₂O is di-tert-butyl dicarbonate; brine is a saturated sodium chloride water solution; BINAP is (±)-2,2′-bis(diphenylphosphino)-1,1′-binaphthalene; BF₃OEt₂ is boron trifluoride diethyl etherate; °C is degrees Celsius; CDCl₃ is deuterated chloroform; CD₃OD is deuterated methanol; (CD₃)₂SO is deuterated dimethyl sulfoxide; CD₃CN is deuterated acetonitrile; cataCXium® A is di(1-adamantyl)-N-butylphosphine; cataCXium® A Pd G3 is mesylate[(di(1-adamantyl)-N-butylphosphine)-2-(2′-amino-1,1′-biphenyl)]palladium(II); CAS is Chemical Abstracts Service; CDI is 1,1′-carbonyldiimidazole; CHCl₃ is chloroform; CO₂ is carbon dioxide; CsHCO₃ is cesium hydrogencarbonate; Cs₂CO₃ is cesium carbonate; CsF is cesium fluoride; CuI is copper(I) iodide; DBU is 1,8-diazabicyclo[5.4.0]undec-7-ene; DCM is dichloromethane; DAST is (diethylamino)sulfurtrifluoride; DCE is 1,2-dichloroethane; DDQ is 2,3-dichloro-5,6-dicyano-p-benzoquinone; DEA is diethylamine; DIEA is N,N-diisopropylethylamine; dioxane is 1,4-dioxane; DMA is N,N-dimethylacetamide; DMAP is 4-(dimethylamino)pyridine; DME is dimethyl ether; DMEDA is 1,2-dimethylethylenediamine; DMAc is dimethylacetamide; DMF is N,N-dimethylformamide; DMSO is dimethyl sulfoxide; EDCI is 1-(3-dimethylaminopropyl)-3-ethylcarbodiimide hydrochloride; ee is enantiomeric excess; EtOAc is ethyl acetate; EtOH is ethanol; g is gram; g/L is grams per liter; GPhos Pd G6 TES is GPhos OAC precatalyst TES; h is hour; HATU is 1-[bis(dimethylamino)methylene]-1H-1,2,3-triazolo[4,5-b]pyridinium 3-oxid hexafluorophosphate; HCl is hydrochloric acid; H₂ is hydrogen; H₂O is water; HOBt is 1-hydroxybenzotriazole hydrate; HOPO is 2-pyridinol 1-oxide; HPLC is high performance liquid chromatography; HR/MS is high resolution mass spectrometry; Hz is hertz; Ir(ppy)₂(dtbbpy)PF₆ is [4,4′-bis(1,1-dimethylethyl)-2,2′-bipyridine-N1,N1′]bis[2-(2-pyridinyl-N)phenyl-C]iridium(II) hexafluorophosphate; KHSO₄ is potassium bisulfate; KHMDS is potassium bis(trimethylsilyl)amide; KF is potassium fluoride; KOH is potassium hydroxide; KOTMS is potassium trimethylsilanolate; K₂CO₃ is potassium carbonate; kg is kilogram; KI is potassium iodide; JosiPhos SL-J009-1 Pd G3 is {(R)-1-[(Sp)-2-(dicyclohexylphosphino)ferrocenyl]ethyldi-tert-butylphosphine}[2-(2′-amino-1,1′-biphenyl)]palladium(II) methanesulfonate; AcOK is potassium acetate; NaBH(OAc)₃ is sodium triacetoxyborohydride; Cbz is benzyloxycarbonyl; K₃PO₄ is tripotassium phosphate; K is kelvin; KH₂PO₄ is potassium phosphate monobasic; L is liter; LC is liquid chromatography; LCMS is liquid chromatography mass spectrometry; lutidine is 2,6-dimethylpyridine; LDA is lithium diisopropylamide; LED is light-emitting diode; LiOH is lithium hydroxide monohydrate; M is molar; LiHMDS is lithium bis(trimethylsilyl)amide; Mel is iodomethane; Mn(TMHD)₃ is tris(2,2,6,6-tetramethyl-3,5-heptanedionato)manganese(III); MeOH is methanol; 4MetBuXPhos Pd G3 is methanesulfonato (2-di-tert-butylphosphino-3,4,5,6-tetramethyl-2′,4′,6′-triisopropyl-1,1-biphenyl)(2′-amino-1,1′-biphenyl-2-yl) palladium(II); mg is milligram; MgSO₄ is magnesium sulfate; MPLC is medium performance liquid chromatography; MHz is megahertz; min is minute; mL is milliliter; mL/min is milliliter per minute; mmol is millimole; mol is mole; mM is millimolar; mm is millimeter; MPa is megapascal; MS is mass spectrometry; MTBE is methyl tertiary-butyl ether; m/z is mass-to-charge ratio; Mn(TMHD)₃ is tris(2,2,6,6-tetramethyl-3,5-heptanedionato)manganese(II); N is normal (concentration); N₂ is nitrogen; NBS is N-bromosuccinimide; NaBH₄ is sodium borohydride; ND is not determined; N-XantPhos Pd G3 is (2′-amino-2-biphenylyl)(methanesulfonato-κO)palladium-4,6-bis(diphenylphosphino)-10H-phenoxazine; NH₃ is ammonia; Na₂CO₃ is sodium carbonate; NaCl is sodium chloride; NaH is sodium hydride; NaHCO₃ is sodium bicarbonate; NaI is sodium iodide; NaOCH₃ is sodium methoxide; NaOH is sodium hydroxide; Na₂SO₃ is sodium sulfite; Na₂SO₄ is sodium sulfate; NaBH₃CN is sodium cyanoborohydride; NaIO₄ is sodium periodate; NaOCN is sodium cyanate; Na₂HPO₄ is sodium phosphate dibasic; NaOtBu is sodium tert-butoxide; n-BuLi is n-butyllithium; ND is not determined; NH₄Cl is ammonium chloride; NH₄HCO₃ is ammonium bicarbonate; NH₄OH is ammonium hydroxide; NMR is nuclear magnetic resonance; NiCl₂·glyme is nickel(II) chloride ethylene glycol dimethyl ether complex; NiBr₂·DME is nickel(II) bromide ethylene glycol dimethyl ether complex; Pd is palladium; Pd/Al₂O₃ is palladium on alumina; Pd(OH)₂/C is palladium hydroxide on carbon; Pt/C is platinum on carbon; Pd-PEPPSI-lHept-CI is (SP-4-1)-[1,3-bis[2,6-bis(1-propylbutyl)phenyl]-4,5-dichloro-1,3-dihydro-2H-imidazol-2-ylidene]dichloro(3-chloropyridine-κN)-palladium; P(t-Bu)₃ Pd G2 is chloro[(tri-tert-butylphosphine)-2-(2-aminobiphenyl)]palladium(II); Pd(dppf)Cl₂ is (1,1′-bis(diphenylphosphino)ferrocene)palladium(II) dichloride or [1,1′-bis(diphenylphosphino)ferrocene]dichloropalladium(II), complex with dichloromethane; Pd/C is palladium on carbon; Pd₂(dba)₃ is tris(dibenzylideneacetone)dipalladium(0); PdCl₂(PPh₃)₂ is bis(triphenylphosphine)palladium(II) dichloride; Pd(OAc)₂ is palladium(II) acetate; Pd(t-Bu₃P)₂ is bis(tri-tert-butylphosphine)palladium(0); PE is petroleum ether; psi is pounds per square inch; quinuclidine is 1-azabicyclo[2.2.2]octane; OsO₄ is osmium tetroxide; sccm is standard cubic centimeters per minute; SFC is super critical fluid chromatography; SOCl₂ is thionyl chloride; TBD is 1,5,7-triazabicyclo[4.4.0]dec-5-ene; TEA is triethylamine; tert-amyl alcohol is 2-methyl-2-butanol; TFA is trifluoroacetic acid; TFAA is trifluoroacetic anhydride; TBAI is tetrabutylammonium iodide; THF is tetrahydrofuran; TLC is thin-layer chromatography; trimethylboroxine is 2,4,6-trimethyl-1,3,5,2,4,6-trioxatriborinane; μm is micrometer; μmol is micromole; rac is racemic referring to the compound including a mixture of compounds having both R and S stereochemistry and is denoted in the structures as “&1”; re/is relative referring to the stereochemistry being either R or S and is denoted in the structures as “or1” or as an asterisk in the named structure if there are two chiral centers in the compound; RhCI(PPh₃)₃ is tris(triphenylphosphine)rhodium(I) chloride; rpm is revolutions per minute; RPLC is reverse-phase liquid chromatography; Ru/Al₂O₃ is ruthenium on aluminum oxide; RuPhos is 2-dicyclohexylphosphino-2′,6′-diisopropoxybiphenyl; RuPhos Pd G3 is (2-dicyclohexylphosphino-2′,6′-diisopropoxy-1,1′-biphenyl)[2-(2′-amino-1,1′-biphenyl)]palladium(II) methanesulfonate; RuPhos Pd G4 is [dicyclohexyl(2′,6′-diisopropoxy-2-biphenylyl)phosphine-KP](methanesulfonatato-κO)[2′-(methylamino-κN)-2-biphenylyl-κC²]palladium; RuCI(PPh₃)₃ Ru/SiO₂ is ruthenium on silica; wt is weight; wt % is weight percent; XPhos Pd G2 is chloro(2-dicyclohexylphosphino-2′,4′,6′-triisopropyl-1,1′-biphenyl)[2-(2′-amino-1,1′-biphenyl)]palladium(II); XPhos Pd G3 is (2-dicyclohexylphosphino-2′,4′,6′-triisopropyl-1,1′-biphenyl)[2-(2′-amino-1,1′-biphenyl)]palladium(II) methanesulfonate; XantPhos Pd G4 is (SP-4-3)-[[5-(diphenylphosphino)-9,9-dimethyl-9H-xanthen-4-yl]diphenylphosphine-κP](methanesulfonato-κO)[2′-(methylamino-κN)[1,1′-biphenyl]-2-yl-κC]- palladium; and Zn is zinc.

### Preparation 1

### Ethyl 1-cyclopropyl-4,5,6,7-tetrahydro-1H-pyrazolo[4,3-c]pyridine-3-carboxylate hydrochloride (P1)

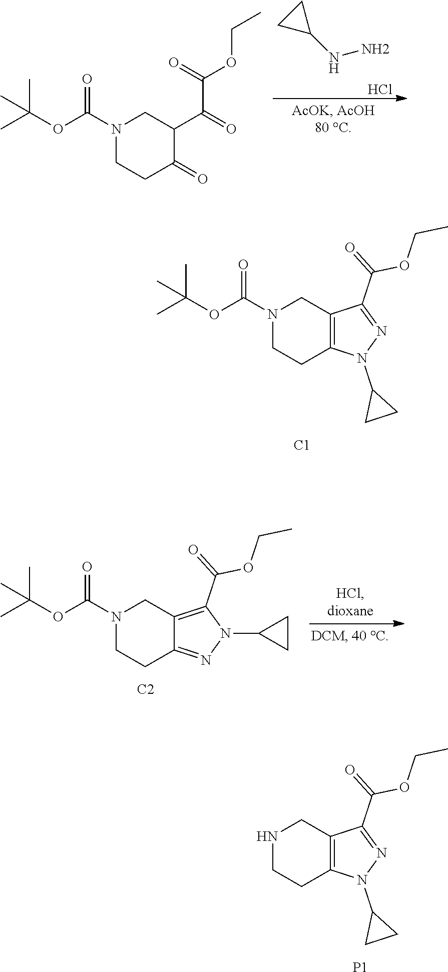

### Step 1. Preparation of 5-(tert-butyl) 3-ethyl 1-cyclopropyl-1,4,6,7-tetrahydro-5H-pyrazolo[4,3-c]pyridine-3,5-dicarboxylate (C1) and 5-(tert-butyl) 3-ethyl 2-cyclopropyl-2,4,6,7-tetrahydro-5H-pyrazolo[4,3-c]pyridine-3,5-dicarboxylate (C2)

A reaction containing cyclopropylhydrazine monohydrochloride (36.3 mg, 0.334 mmol) and AcOK (36.1 mg, 0.367 mmol) in AcOH (1.0 mL) was heated at 80 °C for 10 min then cooled to room temperature. tert-butyl 3-(2-ethoxy-2-oxoacetyl)-4-oxopiperidine-1-carboxylate (CAS: 518990-24-4; 0.100 g, 0.334 mmol) was added to the mixture. The reaction turned into a viscous yellow oil, which was stirred at 80 °C for 1 h and 30 min. The reaction was then diluted with EtOAc and saturated aqueous NaHCO₃. The organic layer was separated and concentrated in vacuo. The residue was purified by column chromatography (silica gel, 0-40% EtOAc: heptane) to provide minor first eluting isomer C2: (11 mg, 27.2% yield) (LC/MS) m/z (M+H)⁺=336.3; followed by major second eluting isomer C1:(18 mg, 44.5% yield) (LC/MS) m/z (M+H)⁺=336.3; ¹H NMR (600 MHz, CDCl₃) δ (ppm): 4.66-4.54 (m, 2H), 4.36 (q, 2H), 3.71 (s, 2H), 3.40-3.34 (m, 1H), 2.84-2.74 (m, 2H), 1.48-1.43 (m, 9H), 1.41-1.34 (m, 3H), 1.21-1.18 (m, 2H), 1.07-1.02 (m, 2H).

### Step 2. Preparation of ethyl 1-cyclopropyl-4,5,6,7-tetrahydro-1H-pyrazolo[4,3-c]pyridine-3-carboxylate hydrochloride (P1)

A solution of C1 (0.200 g, 0.596 mmol) was dissolved in DCM (2.0 mL), then 4M HCl in dioxane (0.130 g, 3.58 mmol) was added, then the reaction was stirred at 40 °C for 25 min. The reaction was concentrated in vacuo to provide P1 (0.168 mg, crude) as a white solid. The solid was used in the next step without further purification. (LC/MS) m/z (M+H)⁺=236.2; ¹H NMR (600 MHz, CD₃OD) δ 4.41-4.33 (m, 4H), 3.61-3.53 (m, 3H), 3.16 (t, 2H), 1.37 (t, 3H), 1.21-1.16 (m, 2H), 1.15-1.09 (m, 2H).

### Preparation 2

### Ethyl 1-isopropyl-4,5,6,7-tetrahydro-1H-pyrazolo[4,3-c]pyridine-3-carboxylate hydrochloride (P2)

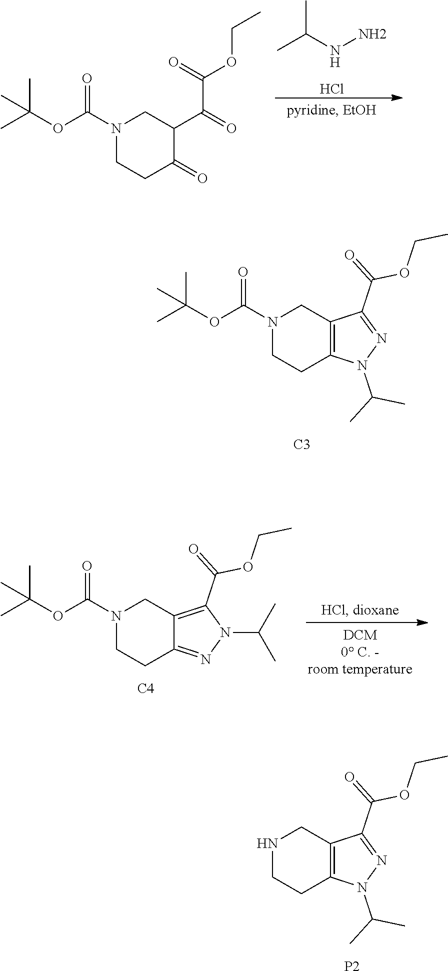

### Step 1. Preparation of 5-(tert-butyl) 3-ethyl 1-isopropyl-1,4,6,7-tetrahydro-5H-pyrazolo[4,3-c]pyridine-3,5-dicarboxylate (C3) and 5-(tert-butyl) 3-ethyl 2-isopropyl-2,4,6,7-tetrahydro-5H-pyrazolo[4,3-c]pyridine-3,5-dicarboxylate (C4)

To a solution of tert-butyl 3-(2-ethoxy-2-oxoacetyl)-4-oxopiperidine-1-carboxylate (CAS: 518990-24-4, 325 g, 1.09 mol) in EtOH (2.3 L) was added isopropylhydrazine hydrochloride (120 g, 1.1 mol) in one portion then pyridine (103 g, 1.30 mol) was added dropwise into the mixture for 10 min at 20-25 °C with ice water bath. The reaction mixture was stirred for 2 h at room temperature. The reaction was carried out in two batches in parallel, were combined, and then concentrated in vacuo. The residue was diluted with EtOAc (5 L) and washed with brine (2×5 L) then dried with Na₂SO₄, filtered, and concentrated in vacuo. The residue was purified by column chromatography (silica gel, EtOAc: PE from 10-50%) to provide C3 and C4. C3 was dissolved in PE (0.8 L) and then cooled to −20 °C. The mixture was stirred for 4 h, resulting solids were filtered, and washed with PE (0.1 L). The filter cake was dried under high vacuum to obtain C3 (414 g, 55.9% yield) as a white solid. (LC/MS) m/z (M+H)⁺=338.1; ¹H NMR (400 MHz, CDCl₃) δ (ppm): 4.59 (s, 2H), 4.48-4.32 (m, 3H), 3.71 (t, 2H), 2.70 (t, 2H), 1.53-1.45 (m, 15H), 1.39-1.34 (m, 3H). C₄ was dissolved in PE (50 mL) then cooled to −20 °C. The mixture was stirred for 4 h, resulting solids were filtered, and washed with PE (20 mL). The filter cake was dried under high vacuum to provide C4 (11.7 g, 1.58% yield) as a white solid. (LC/MS) m/z (M+H)⁺=338.1; ¹H NMR (400 MHz, CDCl₃) δ 5.60-5.43 (m, 1H), 4.60 (s, 2H), 4.33 (q, 2H), 3.68 (s, 2H), 2.75 (s, 2H), 1.50-1.44 (m, 15H), 1.38 (m, 3H).

### Step 2. Preparation of ethyl 1-isopropyl-4,5,6,7-tetrahydro-1H-pyrazolo[4,3-c]pyridine-3-carboxylate hydrochloride (P2)

To a mixture of C₃ (13.5 g, 40.0 mmol) in DCM (60 mL) was added 4N HCl in dioxane (150 mL) in batches at 0 °C. The reaction was stirred at about 15 °C for 16 h and then concentrated in vacuo to provide P2 (10.5 g, crude) as a white solid, used directly in the next step without further purification. LC/MS m/z (M+H)⁺=238.1; ¹H NMR (600 MHz, (CD₃)₂SO) δ (ppm): 9.80 (s, 1H), 4.63-4.51 (m, 1H), 4.28 (q, 2H), 4.18 (s, 2H), 3.36 (s, 2H), 3.02 (t, 2H), 1.38 (d, 6H), 1.29 (t, 3H).

### Preparation 3

### 5-(tert-Butoxycarbonyl)-1-ethyl-4,5,6,7-tetrahydro-1H-pyrazolo[4,3-c]pyridine-3-carboxylic acid (P3)

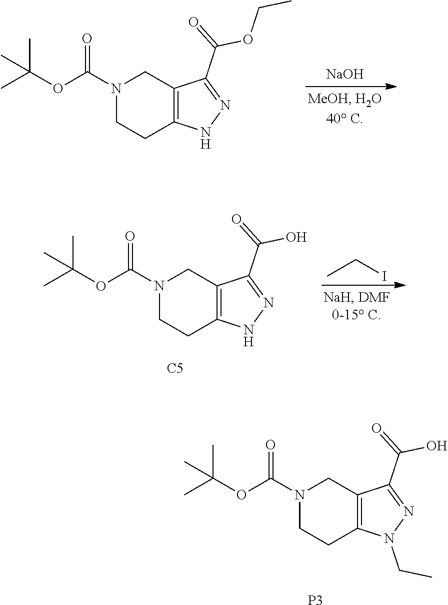

### Step 1. Preparation of 5-(tert-butoxycarbonyl)-4,5,6,7-tetrahydro-1H-pyrazolo[4,3-c]pyridine-3-carboxylic acid (C5)

To a suspension of 5-(tert-butyl) 3-ethyl 1,4,6,7-tetrahydro-5H-pyrazolo[4,3-c]pyridine-3,5-dicarboxylate (CAS: 518990-23-3; 2.00 g, 6.77 mmol) in MeOH (7.7 mL) and H₂O (7.7 mL) was added NaOH (0.542 g, 13.5 mmol). The reaction mixture was stirred at 40 °C for 16 h. The mixture was diluted with H₂O (20 mL), cooled to 0 °C and acidified with 1 M aqueous HCl to pH of about 3. The resulting mixture was filtered, the solid was dried further under a high vacuum and then lyophilized to provide C5 (1.60 g, crude) as a white solid. The solid was used directly in the next step without further purification. (LC/MS) m/z (M+H)⁺=268.1. ¹H NMR (400 MHz, (CD₃)₂SO) δ (ppm): 13.17 (br s, 2H), 4.48 (s, 2H), 3.58 (t, 2H), 2.65 (t, 2H), 1.41 (s, 9H).

### Step 2. Preparation of 5-(tert-butoxycarbonyl)-1-ethyl-4,5,6,7-tetrahydro-1H-pyrazolo[4,3-c]pyridine-3-carboxylic acid (P3)

To the suspension of C5 (0.200 g, 0.748 mmol) in DMF (3.5 mL) was added NaH (89.8 mg, 2.24 mmol, 60%) at 0 °C and stirred for 30 min at 0 °C. A solution of iodoethane (0.140 g, 0.898 mmol) in DMF (0.5 mL) was added at 0 °C then stirred at 15 °C for about 4 h. An additional portion of the solution of iodoethane (23.3 mg, 0.150 mmol) in DMF (0.2 mL) was added to the mixture and stirred at 15 °C for 16 h. The reaction solution was quenched with H₂O (10 mL) and then extracted with EtOAc (2×20 mL). The aqueous layer was acidified with 1 M aqueous HCl to pH ~3 and then extracted with EtOAc (2×30 mL). The combined organic layers were washed with brine (2×30 mL), dried over Na₂SO₄, concentrated in vacuo and then lyophilized to provide P3 (0.173 g, 78.5% yield) as a white solid. (LC/MS) m/z (M+H)⁺=296.1. ¹H NMR (400 MHz, (CD₃)₂SO) δ (ppm): 12.64 (br s, 1H), 4.45 (s, 2H), 4.07 (q, 2H), 3.60 (t, 2H), 2.74-2.65 (m, 2H), 1.41 (s, 9H), 1.31 (t, 3H).

### Preparation 3b

### Ethyl 1-ethyl-4,5,6,7-tetrahydro-1H-pyrazolo[4,3-c]pyridine-3-carboxylate hydrochloride (P3b)

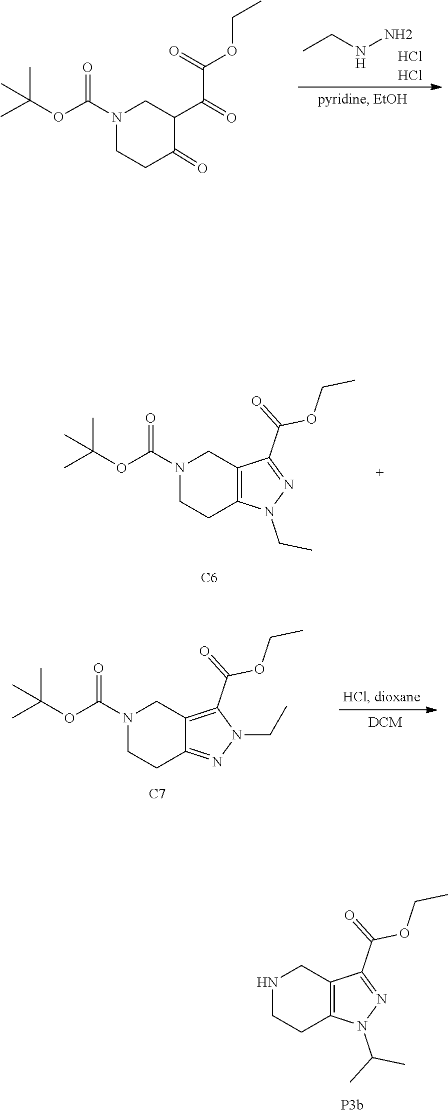

### Step 1. Preparation of 5-(tert-butyl) 3-ethyl 1-ethyl-1,4,6,7-tetrahydro-5H-pyrazolo[4,3-c]pyridine-3,5-dicarboxylate (C₆) and 5-(tert-butyl) 3-ethyl 2-ethyl-2,4,6,7-tetrahydro-5H-pyrazolo[4,3-c]pyridine-3,5-dicarboxylate (C7)

To a solution of tert-butyl 3-(2-ethoxy-2-oxoacetyl)-4-oxopiperidine-1-carboxylate (CAS: 518990-24-4; 97.5 g, 326 mmol) and ethylhydrazine dihydrochloride (56.3 g, 423 mmol) in EtOH (1.6 L) was added pyridine (129 g, 1.63 mol). The reaction mixture was stirred at room temperature for 16 h then concentrated in vacuo. The residue was purified by column chromatography (silica gel, 0-50% THF: PE) to provide the first eluting isomer C₆ (58.0 g) as a white solid; followed by the second eluting isomer C7 (20.6 g, 19.6% yield) as a light-yellow oil. C6 was diluted with EtOAc (200 mL) then stirred at 50 °C for 30 min and cooled to 0 °C which caused white solid precipitate to form. The suspension was filtered, and the filter cake was washed with EtOAc (3×10 mL). The filter cake was collected and concentrated in vacuo to provide C6 (35.4 g, 33.6% yield) as a white solid.

C6: (LC/MS) m/z (M+H)⁺=324.2; ¹H NMR (400 MHz, CDCl₃) δ (ppm): 4.60 (s, 2H), 4.38 (q, 2H), 4.12 (q, 2H), 3.71 (t, 2H), 2.68 (t, 2H), 1.47 (s, 9H), 1.44-1.34 (m, 6H).

C7: (LC/MS) m/z (M+H)⁺=324.3; ¹H NMR (400 MHz, CDCl₃) δ (ppm): 4.65-4.50 (m, 4H), 4.34 (q, 2H), 3.73-3.63 (m, 2H), 2.80-2.67 (m, 2H), 1.48 (s, 9H), 1.43-1.33 (m, 6H).

### Step 2. Preparation of ethyl 1-ethyl-4,5,6,7-tetrahydro-1H-pyrazolo[4,3-c]pyridine-3-carboxylate hydrochloride (P3b)

To a solution of C6 (1.00 g, 3.09 mmol) in DCM (10 mL) was added 2M HCl in dioxane (1.13 g, 30.9 mmol). The reaction mixture was stirred at room temperature for 2.5 h then concentrated in vacuo to provide P3b (0.870 g, crude) as a white solid. The solid was used directly in the nest step without further purification. (LC/MS) m/z (M+H)⁺=224.2. ¹H NMR (400 MHz, CD₃OD) δ (ppm): 4.44-4.32 (m, 4H), 4.20 (q, 2H), 3.56 (t, 2H), 3.09 (t, 2H), 2.80-2.67 (m, 2H), 1.44 (t, 3H), 1.38 (t, 3H).

### Preparation 4

### Ethyl 1-methyl-4,5,6,7-tetrahydro-1H-pyrazolo[4,3-c]pyridine-3-carboxylate hydrochloride (P4)

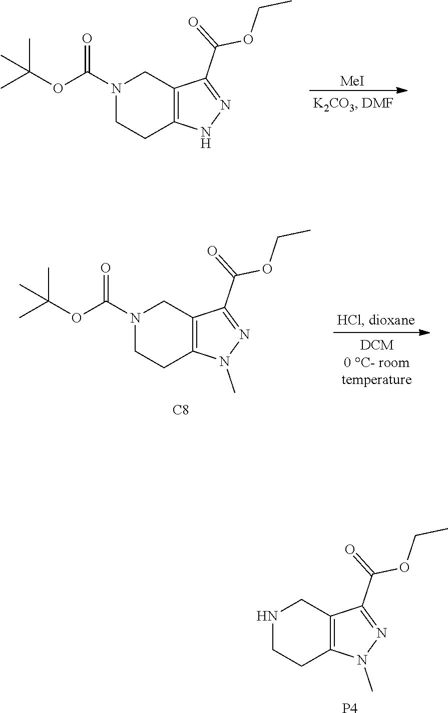

### Step 1. Preparation of 5-(tert-butyl) 3-ethyl 1-methyl-1,4,6,7-tetrahydro-5H-pyrazolo[4,3-c]pyridine-3,5-dicarboxylate (C8)

5-(tert-Butyl) 3-ethyl 1,4,6,7-tetrahydro-5H-pyrazolo[4,3-c]pyridine-3,5-dicarboxylate (CAS: 518990-23-3; 2.00 g, 6.77 mmol) was dissolved in DMF (10.0 mL) then K₂CO₃ (2.81 g, 20.3 mmol) and Mel (0.961 g, 6.77 mmol) were added. The reaction was stirred at room temperature for 3 h and diluted with EtOAc and H₂O. The organic layer was separated and then concentrated in vacuo. The residue was dissolved in DCM and then purified by column chromatography (silica gel, 0-40% EtOAc: heptane) to provide C8 (0.825 g, 39.4% yield) as a clear oil. (LC/MS) m/z (M+H)⁺=310.3; ¹H NMR (600 MHz, CDCl₃) δ (ppm): 4.61 (s, 2H), 4.35 (q, 2H), 4.14 (s, 3H), 3.68 (s, 2H), 2.74 (s, 2H), 1.49 (s, 9H), 1.41-1.37 (m, 3H).

### Step 2. Preparation of ethyl 1-methyl-4,5,6,7-tetrahydro-1H-pyrazolo[4,3-c]pyridine-3-carboxylate hydrochloride (P4)

C8 (2.00 g, 6.46 mmol) was dissolved in 4M HCl in dioxane (40.0 mL) at 0 °C and then stirred at room temperature for 16 h. The reaction was concentrated in vacuo to provide P4 (1.30 g, 81.9% yield) as a yellow solid. This was used in the next step without further purification. (LC/MS) m/z (M+H)⁺=210.3. ¹H NMR (400 MHz, CDCl₃) δ (ppm): 10.23 (br s, 1H), 4.50-4.32 (m, 4H), 3.86 (s, 3H), 3.55-3.46 (m, 2H), 3.13 (t, 2H), 1.40 (t, 3H).

### Preparation 5

### Ethyl 3-cyclopropyl-5,6,7,8-tetrahydroimidazo[1,5-a]pyrazine-1-carboxylate hydrochloride (P5)

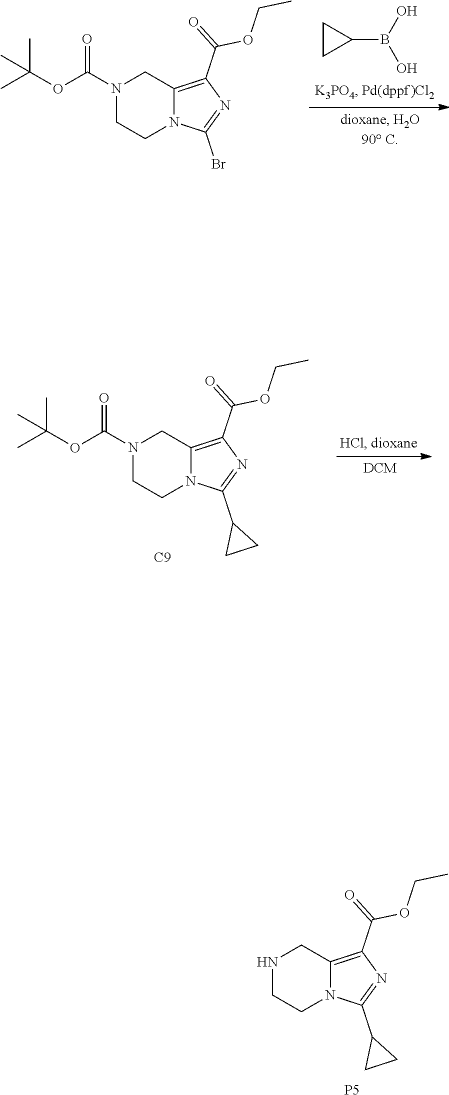

### Step 1: Preparation of 7-(tert-butyl) 1-ethyl 3-cyclopropyl-5,6-dihydroimidazo[1,5-a]pyrazine-1,7(8H)-dicarboxylate (C9)

To a solution of 7-(tert-butyl) 1-ethyl 3-bromo-5,6-dihydroimidazo[1,5-a]pyrazine-1,7(8H)-dicarboxylate (CAS: 2108354-93-2; 0.578 g, 1.54 mmol) and cyclopropylboronic acid (CAS: 411235-57-9, 0.398 g, 4.63 mmol) in dioxane (8.0 mL) and H₂O (1.0 mL) was added K₃PO₄ (0.984 g, 4.63 mmol) and Pd(dppf)Cl₂ (0.226 g, 0.309 mmol) at room temperature under nitrogen gas. The reaction was stirred at 90 °C for 16 h. The resulting solution was concentrated in vacuo to give a solid that was purified by column chromatography (silica gel, 0-95% EtOAc: PE) to provide C9 (0.400 g, 77.2% yield) as a yellow gum. (LC/MS) m/z (M+H)⁺=336.1. ¹H NMR (400 MHz, CD₃OD) δ (ppm): 4.84 (s, 2H), 4.30 (q, 2H), 4.14-4.04 (m, 2H), 3.86 (t, 2H), 1.94-1.86 (m, 1H), 1.53-1.48 (m, 9H), 1.36 (t, 3H), 1.00-0.96 (m, 4H).

### Step 2. Preparation of ethyl 3-cyclopropyl-5,6,7,8-tetrahydroimidazo[1,5-a]pyrazine-1-carboxylate hydrochloride (P5)

To a solution of C9 (0.300 g, 0.894 mmol) in DCM (5.0 mL) was added 2M HCl in dioxane (0.261 g, 7.16 mmol) and reaction stirred at room temperature for 16 h. The resulting suspension was concentrated in vacuo to provide P5 (0.243 g, crude) as a white solid, used directly in the next step without further purification. ¹H NMR (400 MHz, (CD₃)₂SO) δ (ppm): 10.17 (s, 1H), 4.50 (s, 2H), 4.36 (t, 2H), 4.25 (q, 2H), 3.62-3.57 (m, 2H), 2.13-2.03 (m, 1H), 1.30-1.23 (m, 3H), 1.04-0.98 (m, 2H), 0.97-0.91 (m, 2H).

### Preparation 6

### rac-Ethyl (R)-1,7-dimethyl-4,5,6,7-tetrahydro-1H-pyrazolo[4,3-c]pyridine-3-carboxylate hydrochloride (P6)

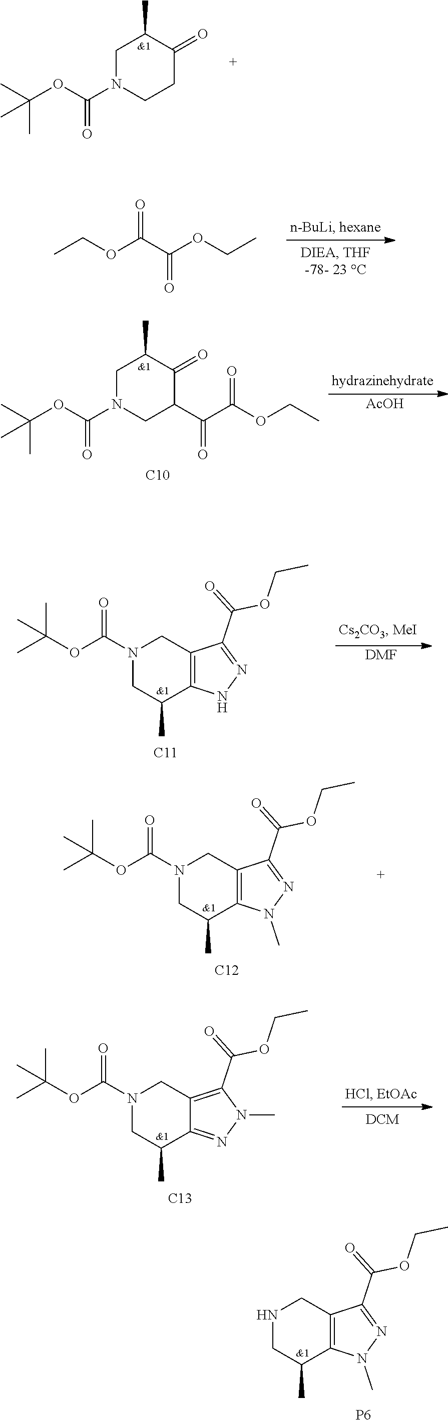

### Step 1: Preparation of rac-tert-butyl (5R)-3-(2-ethoxy-2-oxoacetyl)-5-methyl-4-oxopiperidine-1-carboxylate (C10)

A solution of diisopropylamine (4.2 g, 41 mmol) in THF (19.0 mL) was cooled to −78 °C before 2.5M n-BuLi in hexane (2.5 g, 39 mmol) was added. After the addition, the reaction mixture was warmed to 0 °C and stirred for 15 min. The reaction was then cooled to −78 °C and a solution of rac-tert-butyl (R)-3-methyl-4-oxopiperidine-1-carboxylate (CAS:181269-69-2, 7.0 g, 33 mmol) in THF (81.0 mL) was slowly added while maintaining the temperature below −67 °C. The mixture was stirred at −78 °C for about 1 h and 30 min before diethyl oxalate (4.8 g, 33 mmol) was added to the reaction solution. The resulting mixture was allowed to warm to room temperature and then stirred for 2 h. The mixture was neutralized with 1 N HCl (80 mL) and extracted with EtOAc (2×200 mL). The combined organic layers were washed with brine (400 mL), dried over Na₂SO₄ and concentrated in vacuo. The residue was purified by column chromatography (silica gel, 0-10% EtOAc: PE) to provide C10 (4.8 g, 47% yield) as a yellow oil. (LC/MS) m/z (M+H)⁺=314.4. ¹H NMR (400 MHz, CDCl₃) δ (ppm): 4.51-4.30 (m, 3H), 3.91-3.65 (m, 1H), 3.34-3.12 (m, 1H), 2.76-2.60 (m, 1H), 1.51-1.44 (m, 9H), 1.41-1.34 (m, 3H), 1.28-1.20 (m, 3H).

### Step 2: Preparation of rac-5-(tert-butyl) 3-ethyl (R)-7-methyl-1,4,6,7-tetrahydro-5H-pyrazolo[4,3-c]pyridine-3,5-dicarboxylate (C11)

To a solution of C10 (3.55 g, 11.3 mmol) in AcOH (14.2 mL), hydrazine hydrate was added (2.09 g, 65% wt, 27.2 mmol). During the addition, the reaction mixture became exothermic to 65 °C. The reaction mixture was stirred for 1 h then concentrated in vacuo. The residue was diluted with H₂O followed by saturated aqueous NaHCO₃ (200 mL). The mixture was extracted with EtOAc (2×200 mL). The combined organic layers were dried with Na₂SO₄, filtered, concentrated in vacuo, and dried further under a high vacuum to provide C11 (3.45 g, crude) as a pale-yellow solid, used directly in the next step without further purification. (LC/MS) m/z (M+H)⁺=310.40.

### Step 3: Preparation of rac-5-(tert-butyl) 3-ethyl (R)-1,7-dimethyl-1,4,6,7-tetrahydro-5H-pyrazolo[4,3-c]pyridine-3,5-dicarboxylate (C12) and rac-5-(tert-butyl) 3-ethyl (R)-2,7-dimethyl-2,4,6,7-tetrahydro-5H-pyrazolo[4,3-c]pyridine-3,5-dicarboxylate (C13)

To a solution of C11 (3.45 g, 11.2 mmol) in DMF (28.6 mL), Cs₂CO₃ (3.65 g, 11.2 mmol) was added, followed by Mel (4.80 g, 33.5 mmol). The reaction mixture was stirred for 16 h at room temperature, diluted with H₂O (100 mL) and extracted with EtOAc (3×50 mL). The combined organic layer was washed with saturated aqueous NH₄Cl (2×100 mL) and H₂O (2×100 mL). The organic layer was dried over Na₂SO₄, filtered, and then concentrated in vacuo. The residue was purified by column chromatography (silica gel, 10-100% EtOAc: heptane) to provide C13 (2.12 g, 58.9% yield) and C12 (0.999 g, 27.7% yield).

C13: (LC/MS) m/z (M+H)⁺=324.1. ¹H NMR (400 MHz, CDCl₃) δ (ppm): 4.74-4.41 (m, 2H), 4.38-4.27 (m, 2H), 4.17-4.10 (m, 3H), 3.98-3.65 (m, 1H), 3.36-2.88 (m, 2H), 1.48 (s, 9H), 1.38 (t, 3H), 1.26 (d, 3H).

C12: (LC/MS) m/z (M+H)⁺=324.1. ¹H NMR (400 MHz, CDCl₃) δ (ppm): 5.18-4.87 (m, 1H), 4.43-4.30 (m, 2H), 4.27-3.99 (m, 2H), 3.85 (s, 3H), 3.23-2.87 (m, 2H), 1.51-1.43 (m, 9H), 1.41-1.33 (m, 3H), 1.24 (d, 3H).

### Step 4: Preparation of rac-ethyl (R)-1,7-dimethyl-4,5,6,7-tetrahydro-1H-pyrazolo[4,3-c]pyridine-3-carboxylate hydrochloride (P6)

To a solution of C12 (0.536 g, 1.66 mmol) in DCM (9.7 mL) was added 1 M HCl in EtOAc (5.0 mL) at room temperature. The reaction was stirred at room temperature for 16 h. An additional portion of 1 M HCl in EtOAc (5.0 mL) was added at room temperature then stirred for 1 h. The reaction was concentrated in vacuo to provide P6 (0.426 g, 99.1% yield) as an off-white solid. This was used in the next step without further purification. (LC/MS) m/z (M+H)⁺=224.2. ¹H NMR (400 MHz, CDCl₃) δ (ppm): 10.58-10.31 (m, 1H), 10.18-9.87 (m, 1H), 4.58-4.49 (m, 1H), 4.46-4.32 (m, 3H), 3.92 (s, 3H), 3.51-3.25 (m, 3H), 1.57-1.53 (m, 3H), 1.40 (t, 3H).

### Preparation 7

### rac-Methyl (R)-3,5-dimethyl-5,6,7,8-tetrahydroimidazo[1,5-a]pyrazine-1-carboxylate hydrochloride (P7)

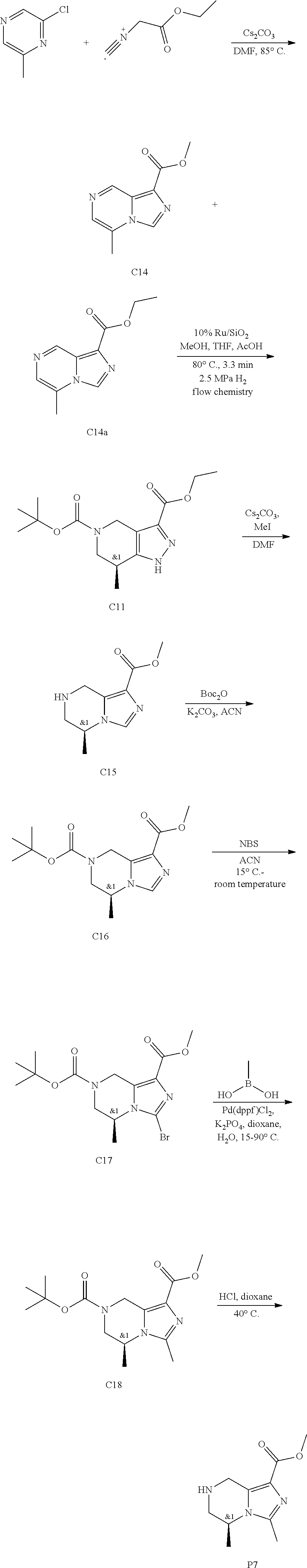

### Step 1. Preparation of methyl 5-methylimidazo[1,5-a]pyrazine-1-carboxylate (C14) and ethyl 5-methylimidazo[1,5-a]pyrazine-1-carboxylate (C14a)

To a mixture of 2-chloro-6-methylpyrazine (CAS: 38557-71-0, 15.0 g, 117 mmol) in DMF (150.0 mL) was added Cs₂CO₃ (57.0 g, 175 mmol) and ethyl 2-isocyanoacetate (CAS: 2999-46-4, 26.6 g, 233 mmol). The reaction was heated to 85 °C and stirred for 16 h. The light brown reaction mixture was filtered then the solid was washed with MeOH. The filtrate was concentrated in vacuo. The brown residue was purified by column chromatography (silica gel, 0-100% EtOAC: PE then 2% MeOH: EtOAc). Transesterification occurred during workup and purification to generate C₁₋₄ (9.50 g, 42.6% yield) as a light brown solid (LC/MS) m/z (M+H)⁺=192.1 and a mixture of (2:1, C14a:C14) (1.1 g, 4.59% yield) (LC/MS) m/z (M+H)⁺=206.1. C14 was brought forward to the next step. C14: ¹H NMR (400 MHz, (CD₃)₂SO) δ (ppm): 9.32 (s, 1H), 8.68 (s, 1H), 7.74 (s, 1H), 3.90 (s, 3H), 2.63 (s, 3H).

### Step 2. Preparation of rac-methyl (R)-5-methyl-5,6,7,8-tetrahydroimidazo[1,5-a]pyrazine-1-carboxylate (C15)

The reaction was carried out via flow chemistry. C14 (2.90 g, 15.2 mmol) was suspended in MeOH (50.0 mL), THF (50.0 mL) and AcOH (0.6 mL) then heated to 50 °C to dissolve to a solution. The solution was pumped with a flow rate of 0.3 mL/min and the H₂ had a rate of 30 mL/min. The solution passed through the fixed bed (6.350 (¼″) mm) packed with granular catalyst 10% Ru/SiO₂ (15.3 g, 1.52 mmol) with a 5.0 mL volume and was hydrogenated for 3.3 min at 80 °C via flow at 2.5 MPa H₂. The light-yellow reaction was concentrated in vacuo then dried in high vacuum to provide C15 (2.96 g, crude) as a light brown oil. This was used in the next step without further purification. ¹H NMR (400 MHz, CDCl₃) δ (ppm): 7.60 (s, 1H), 4.43 (d, 1H), 4.27-4.15 (m, 2H), 3.87 (s, 3H), 3.32 (dd, 1H), 2.84 (dd, 1H), 2.68 (s, 1H), 1.53 (d, 3H).

### Step 3. Preparation of rac-7-(tert-butyl) 1-methyl (R)-5-methyl-5,6-dihydroimidazo[1,5-a]pyrazine-1,7(8H)-dicarboxylate (C16)

To a solution of C15 (2.96 g, 15.2 mmol) in ACN (60.0 mL) was added Boc₂O (4.97 g, 22.8 mmol) and K₂CO₃ (6.29 g, 45.5 mmol) at room temperature. The reaction was stirred at room temperature for 16 h. The light brown reaction mixture was filtered then concentrated in vacuo. The brown residue was purified by column chromatography (silica gel; 0-100% EtOAc: PE) to provide C16 (3.02 g, 67.5% yield) as an off-white solid. (LC/MS) m/z (M+H)⁺=296.1. ¹H NMR (400 MHz, CDCl₃) δ (ppm): 7.53 (s, 1H), 5.01-4.77 (m, 2H), 4.33-4.21 (m, 1H), 3.89 (s, 3H), 3.55-3.34 (m, 2H), 1.53-1.49 (m, 12H).

### Step 4. Preparation of rac-7-(tert-butyl) 1-methyl (R)-3-bromo-5-methyl-5,6-dihydroimidazo[1,5-a]pyrazine-1,7(8H)-dicarboxylate (C17)

To a solution of C16 (3.02 g, 10.2 mmol) in ACN (50.0 mL) was added NBS (2.73 g, 15.4 mmol) at 15 °C. The reaction was stirred at room temperature for 16 h. The light brown reaction mixture was concentrated in vacuo. The brown residue was purified by column chromatography (silica gel, 0-100% EtOAc: PE) which gave an impure C17 as a light brown solid. The solid was triturated with EtOAc: PE (1:5). The solid was filtered then rinsed with EtOAc: PE (5:1) to provide C17 (1.32 g, 34.4% yield) as a white solid. (LC/MS) m/z (M+2H)⁺=376.0. ¹H NMR (400 MHz, CDCl₃) δ (ppm): 5.62-5.23 (m, 1H), 4.54-4.22 (m, 3H), 3.93-3.86 (m, 3H), 3.48-3.16 (m, 1H), 1.51 (s, 9H), 1.43 (d, 3H).

### Step 5. Preparation of rac-7-(tert-butyl) 1-methyl (R)-3,5-dimethyl-5,6-dihydroimidazo[1,5-a]pyrazine-1,7(8H)-dicarboxylate (C18)

To a solution of C17 (1.30 g, 3.47 mmol) and methylboronic acid (0.624 g, 10.4 mmol) in dioxane (28.0 mL) and H₂O (7.0 mL) was added K₃PO₄ (2.21 g, 10.4 mmol) then Pd(dppf)Cl₂ (0.519 g, 0.695 mmol) at 15 °C under nitrogen. The reaction was heated to 90 °C for 16 h. The brown reaction mixture was extracted with EtOAc (30 mL). The organic phase was dried over Na₂SO₄ and concentrated in vacuo. The brown residue was purified by column chromatography (silica gel, 0-100% EtOAc: PE then 2% MeOH: EtOAc) then dried by high vacuum to provide C18 (0.460 g, 42.8%) as a light brown solid. (LC/MS) m/z (M+H)⁺=310.2. ¹H NMR (400 MHz, CDCl₃) δ (ppm): 5.51-5.15 (m, 1H), 4.52-4.14 (m, 3H), 3.87 (s, 3H), 3.32-3.10 (m, 1H), 2.39 (s, 3H), 1.50 (s, 9H), 1.36 (d, 3H).

### Step 6. Preparation of rac-methyl (R)-3,5-dimethyl-5,6,7,8-tetrahydroimidazo[1,5-a]pyrazine-1-carboxylate hydrochloride (P7)

To a solution of C18 (140 mg, 0.452 mmol) in DCM (4.0 mL) was added 2M HCl in dioxane (8.0 mL) at room temperature. The reaction was heated to 40 °C and stirred for 2 to 5 h. The light brown reaction mixture was concentrated in vacuo and dried further under high vacuum to provide P7 (0.111 g, crude) as a light brown solid. The solid was used in the next step without further purification.

### Preparation 8

### (S)-5-(tert-Butoxycarbonyl)-7-methyl-4,5,6,7-tetrahydro-1H-pyrazolo[4,3-c]pyridine-3-carboxylic acid (P8)

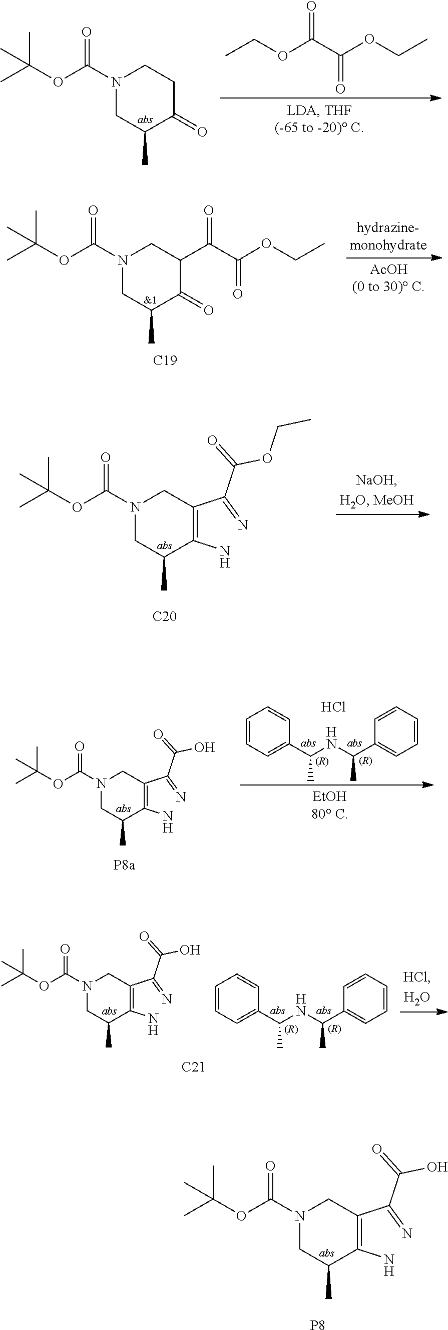

### Step 1. Preparation of rac-tert-butyl (5S)-3-(2-ethoxy-2-oxoacetyl)-5-methyl-4-oxopiperidine-1-carboxylate (C19)

Under a nitrogen balloon at −65 °C, LDA (44.4 g, 0.414 mol, 207 mL) was added to THF (350 mL) then a solution of tert-butyl (S)-3-methyl-4-oxopiperidine-1-carboxylate (CAS: 2092486-33-2; 88.4 g, 0.414 mol) in THF (350 mL) was added dropwise over 1 h to keep the inner temperature below −65 °C. The reaction mixture was stirred at −65 °C for 1 h before a solution of diethyl oxalate (CAS: 95-92-1; 60.6 g, 0.414 mol) in THF (350 mL) was added dropwise at −65 °C. After the addition, the reaction mixture was stirred at −65 °C for 1 h, then warmed to −20 °C and stirred for 40 min. At 0 °C under nitrogen gas, the reaction mixture was quenched with 1 M aqueous KHSO₄ solution (1415 mL), which caused a white precipitate to form. The suspension was filtered, and then the filtrate was extracted with EtOAc (3×800 mL). The combined organic layer was washed with brine (800 mL), dried over Na₂SO₄, filtered then concentrated in vacuo to provide C19 (129 g, crude) as a yellow oil. The oil was used directly in the next step without further purification. (LCMS) m/z (M-tert-butyl group)+=257.9. ¹H NMR (400 MHz, (CD₃)₂SO) δ (ppm): 12.21 (br s, 1H), 4.47-4.17 (m, 3H), 4.06-3.47 (m, 2H), 3.29-3.17 (m, 1H), 2.59-2.53 (m, 1H), 1.45-1.37 (m, 9H), 1.28-1.21 (m, 3H), 1.18-0.95 (m, 3H).

### Step 2. Preparation of enriched 5-(tert-butyl) 3-ethyl (S)-7-methyl-1,4,6,7-tetrahydro-5H-pyrazolo[4,3-c]pyridine-3,5-dicarboxylate (C20)

At 0 °C, to a suspension of C19 (139 g, 0.443 mol) in AcOH (552 mL) was added hydrazine monohydrate (62.7 g, 1.06 mol) which caused the internal temperature to increase to −30 °C. The reaction mixture was stirred at room temperature under nitrogen gas for 1 h then was diluted with H₂O (500 mL) and saturated NaHCO₃ (1000 mL). The diluted suspension was extracted with EtOAc (2×800 mL). The combined organic layer was dried over Na₂SO₄, filtered then concentrated in vacuo to give a yellow gum. The gum was purified by column chromatography (silica gel, (1:1) EtOAc: PE) to give the enriched S-enantiomer C20 (123 g, 89.7% yield) as a yellow gum. (LCMS) m/z (M+H)⁺=310.0. ¹H NMR (400 MHz, (CD₃)₂SO) δ (ppm): 13.71-13.28 (m, 1H), 4.63-4.18 (m, 4H), 3.86-3.59 (m, 1H), 3.12-2.80 (m, 2H), 1.40-1.38 (m, 9H), 1.30-1.24 (m, 3H), 1.17-1.13 (m, 3H). The chiral purity was determined using SFC analytical method on Chiralpak AD-3 100 mm×4.6 mm×3 μm column; Mobile phase A: CO₂/Mobile phase B: EtOH with 0.2% of NH₃; gradient: 5-40% of Mobile Phase B over 3 min then held at 5% of Mobile phase B for 1 min; backpressure: 1500 psi; flow rate: 2.8 mL/min; column temperature: 35 °C to provide C20: major peak ((SFC-MS) m/z (M-tert-butyl group)+=254.04 at retention time: 2.218 min, 94% ee).

### Step 3. Preparation of enriched (S)-5-(tert-butoxycarbonyl)-7-methyl-4,5,6,7-tetrahydro-1H-pyrazolo[4,3-c]pyridine-3-carboxylic acid (P8a)

To a solution of C20 (124 g, 0.400 mol) in MeOH (452 mL) and H₂O (452 mL) was added NaOH (32.0 g, 0.800 mol) then stirred at room temperature for 24 h. The reaction mixture was diluted with H₂O (200 mL) then cooled to 0 °C before the diluted reaction was acidified to pH-3 with 1 M aqueous HCl solution. The acidic reaction mixture was filtered then the filter cake was collected and lyophilized to give the enriched S-enantiomer P8a (88.6. g, crude) as a white solid. The solid was used directly in the next step without further purification. (LCMS) m/z (M+H)⁺=282.0. ¹H NMR (400 MHz, (CD₃)₂SO) δ (ppm): 13.22 (br s, 1H), 4.66-4.34 (m, 2H), 3.86-3.60 (m, 1H), 3.12-2.82 (m, 2H), 1.41 (s, 9H), 1.17 (d, 3H). The chiral purity was determined using SFC analytical method on Chiralpak IG-3 100 mm×4.6 mm×3 μm column; Mobile phase A: CO₂/Mobile phase B: 50% MeOH with 0.2% of NH₃; backpressure: 1500 psi; flow rate: 2.8 mL/min; column temperature: 35 °C to provide P8a: major peak ((SFC-MS) m/z (M-tert-butyl group)+=226.03 at retention time: 0.784 min, 96% ee)

### Step 4. Preparation of (S)-5-(tert-butoxycarbonyl)-7-methyl-4,5,6,7-tetrahydro-1H-pyrazolo[4,3-c]pyridine-3-carboxylic acid (+)-bis[(R)-1-phenylethyl]amine salt (C21)

The reaction was conducted in six batches in parallel then combined. To a mixture of P8a (0.180 kg, 0.640 mol) in EtOH (3.4 L) was added (+)-bis[(R)-1-phenylethyl]amine (CAS: 23294-41-9; 144 g, 0.640 mol) in one portion at room temperature to form the first batch. The reaction mixture of the first batch was stirred at room temperature for 10 min then white precipitate formed. The suspension of the first batch was stirred at 80 °C for 1 h to form a clear solution then stirred at room temperature for 20 h. The white suspension of the first batch was filtered then the filter cake was washed with EtOH (3×300 mL).

The filter cake from the first batch was collected then combined with the filter cakes from the additional five batches of the same reaction utilizing P8a (0.180 kg, 0.640 mol). The combined filter cake was dried in an oven at 50 °C for 18 h to provide C21 (1.42 kg, crude) as a white solid. The solid was used directly in the next step without further purification. (LCMS) m/z (M+H)⁺=282.1. NMR (400 MHz, CD₃OD) δ (ppm): 7.46-7.36 (m, 6H), 7.29-7.23 (m, 4H), 4.76-4.53 (m, 2H), 3.95-3.86 (m, 2H), 3.83-3.70 (m, 1H), 3.25-3.18 (m, 1H), 2.99-2.89 (m, 1H), 1.52 (dd, 6H), 1.48 (s, 9H), 1.25 (d, 3H). The chiral purity was determined using SFC analytical method on Chiralpak IG 50 mm×4.6 mm×3 μm column; Mobile phase A: CO₂/Mobile phase B: MeOH with 0.05% of DEA; gradient: 20-40% of Mobile phase B over 1.5 min and hold 40% of Mobile phase B for 1.0 min then 20% of Mobile Phase B for 0.5 min; flow rate of 4.0 mL/min; backpressure: 1500 psi; column temperature: 35 °C to provide C21: peak 1 (retention time: 1.019 min, 100% ee).

### Step 5. Preparation of (S)-5-(tert-butoxycarbonyl)-7-methyl-4,5,6,7-tetrahydro-1H-pyrazolo[4,3-c]pyridine-3-carboxylic acid (P8)

To a suspension of C21 (48.2 g, 95.1 mmol) in H₂O (482 mL) was added 1M aqueous HCl solution (99 mL) to pH-1. The reaction mixture was stirred at room temperature for 1 h then filtered. The filter cake was collected then lyophilized to give P8 (24.3 g, 90.9% yield) as a white solid. The solid was used directly in the next step without further purification. (LCMS) m/z (M+H)⁺=282.0. ¹H NMR (400 MHz, (CD₃)₂SO) δ (ppm): 13.15 (br s, 1H), 4.63-4.47 (m, 1H), 4.44-4.33 (m, 1H), 3.84-3.61 (m, 1H), 3.19-3.00 (m, 1H), 2.94-2.83 (m, 1H), 1.41 (s, 9H), 1.17 (d, 3H). The chiral purity was determined using SFC analytical method on Chiralpak IG-3 100 mm×4.6 mm×3 μm column; Mobile phase A: 50% of CO₂/Mobile phase B: 50% of MeOH with 0.2% NH₃; backpressure: 1500 psi; flow rate: 2.8 mL/min; column temperature: 35 °C to provide P8: peak 1 ((SFC-MS) m/z (M-tert-butyl group)+=226.00 at retention time: 0.757 min, 99% ee). [α]²⁸_{D}=+11.351 (c=1.5 (g/L in MeOH).

### Preparation 9

### (S)-1-Isopropyl-7-methyl-4,5,6,7-tetrahydro-1H-pyrazolo[4,3-c]pyridine-3-carboxylic acid hydrochloride (P9)

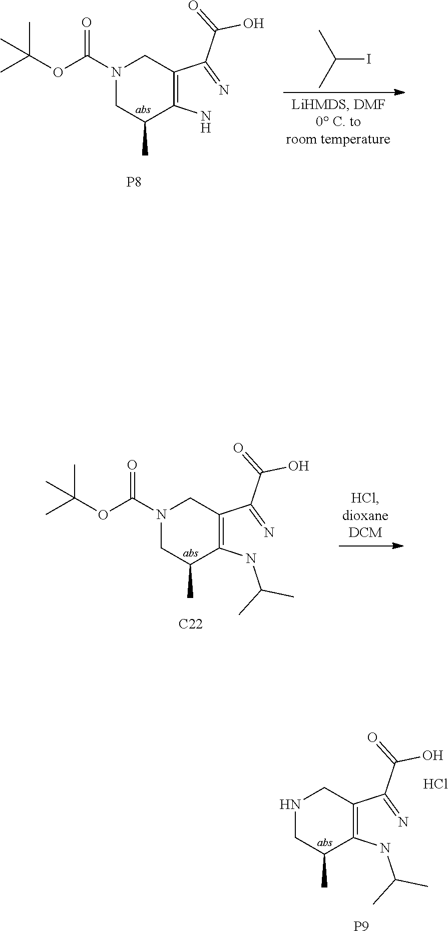

### Step 1. Preparation of (S)-5-(tert-butoxycarbonyl)-1-isopropyl-7-methyl-4,5,6,7-tetrahydro-1H-pyrazolo[4,3-c]pyridine-3-carboxylic acid (C22)

At 0 °C under nitrogen gas, to a solution of P8 (12.4 g, 44.1 mmol) in DMF (220 mL) was added 1 M LiHMDS (132 mL). The reaction mixture was warmed to room temperature and stirred for 2 h before cooling the reaction mixture again to 0 °C. To the cooled reaction mixture was added 2-iodopropane (11.2 g, 66.1 mmol) then the suspension was warmed to room temperature and stirred for 48 h. The reaction mixture was cooled to 0 °C and another portion of 2-iodopropane (1.12 g, 6.61 mmol) was added. The suspension was warmed to room temperature and stirred for 48 h. The reaction mixture was cooled to 0 °C then quenched with H₂O (500 mL). The aqueous phase was washed with EtOAc (2×200 mL) then the combined organic layer was discarded. The aqueous layer was acidified with 1 M aqueous HCl to pH-3 then extracted with EtOAc (2×200 mL). The combined organic layer was washed with brine (2×300 mL), dried over Na₂SO₄ and concentrated in vacuo to give C22 (13.8 g, 96.8% yield) as a yellow solid. (LCMS) m/z (M+H)⁺=324.0. ¹H NMR (400 MHz, (CD₃)₂SO) δ (ppm): 12.60 (br s, 1H), 4.99-4.73 (m, 1H), 4.57-4.41 (m, 1H), 4.19-3.86 (m, 2H), 3.17-3.00 (m, 2H), 1.42-1.36 (m, 15H), 1.17-1.08 (m, 3H). The chiral purity was determined using SFC analytical method on (S,S) Whelk-01, 150 mm×4.6 mm×3.5 μm column; Mobile phase A: CO₂/Mobile phase B: MeOH (0.2% isopropylamine); gradient: 10-50% of Mobile phase B in 6.00 min then held at 10% of Mobile phase B for 2.00 min; backpressure: 2000 psi; flow rate: 1.5 mL/min; column temperature: 35 °C to provide C22: peak 1 (retention time: 2.352 min, 100% ee). [α]³⁴_{D}=−100.10 (c=5.4 (g/L) in ACN).

### Step 2. Preparation of (S)-1-isopropyl-7-methyl-4,5,6,7-tetrahydro-1H-pyrazolo[4,3-c]pyridine-3-carboxylic acid hydrochloride (P9)

To a solution of C22 (12.7 g, 39.3 mmol) in DCM (40 mL) was added 2M HCl in dioxane (500 mL). The reaction mixture was stirred at room temperature for 5 h then was concentrated in vacuo to provide P9 (9.40 g, crude) as a white solid. The solid was used directly in the next step without further purification. (LCMS) m/z (M+H)⁺=224.2. ¹H NMR (400 MHz, CD₃OD) δ (ppm): 4.68-4.58 (m, 1H), 4.45 (d, 1H), 4.31 (d, 1H), 3.62-3.38 (m, 3H), 1.54 (d, 3H), 1.48 (d, 3H), 1.44 (d, 3H). The chiral purity was determined using SFC analytical method on Chiralcel OX-3 100 mm×4.6 mm×3 μm column; Mobile phase A: CO₂/Mobile phase B: MeOH containing 0.05% of DEA; gradient: 5-40% of Mobile Phase B in 3.0 min then held at 40% of Mobile phase B for 0.9 min then held at 5% of Mobile phase B for 0.1 min; backpressure: 100 bar; flow rate: 2.8 mL/min; column temperature: 40 °C to provide P9: peak 1 (retention time: 3.037 min, 100% ee). [α]³³_{D}=−7.212 (c=1.5 (g/L) in MeOH).

### Preparation 10

### (S)-1-Ethyl-7-methyl-4,5,6,7-tetrahydro-1H-pyrazolo[4,3-c]pyridine-3-carboxylic acid hydrochloride (P10)

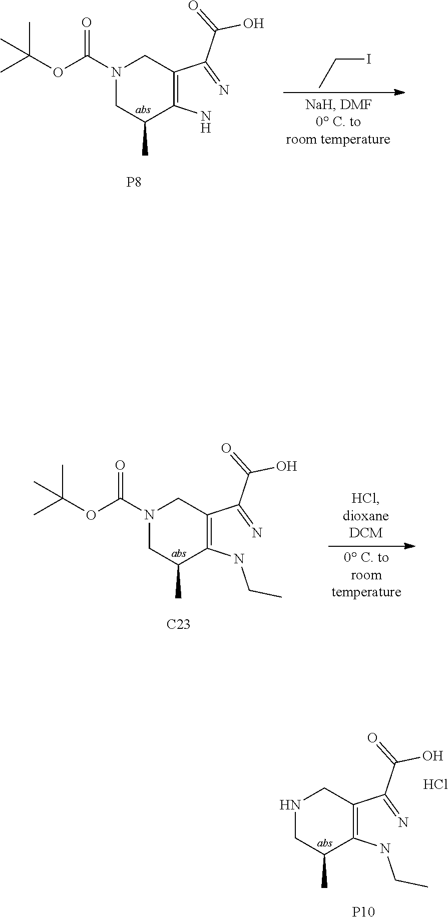

### Step 1. Preparation of (S)-5-(tert-butoxycarbonyl)-1-ethyl-7-methyl-4,5,6,7-tetrahydro-1H-pyrazolo[4,3-c]pyridine-3-carboxylic acid (C23)

At 0 °C under nitrogen gas, to a solution of P8 (10.0 g, 35.5 mmol) in DMF (190 mL) was added NaH (4.27 g, 107 mmol). The reaction mixture was stirred 0 °C for 30 min then a solution of iodoethane (6.65 g, 42.7 mmol) in DMF (10 mL) was added. The suspension was warmed to room temperature and stirred for 3 h then quenched with H₂O (150 mL). The aqueous phase was extracted with EtOAc (2×150 mL) then the combined organic layer was discarded. The aqueous phase was acidified with 1 M aqueous HCl to pH-3 then extracted again with EtOAc (150 mL×3). The combined organic layer was washed with brine (2×100 mL), dried over Na₂SO₄ and concentrated in vacuo to give a yellow solid. The solid was suspended in (1:10) EtOAc: PE (25 mL) and stirred at room temperature for 30 min. The suspension was filtered, and the filter cake was collected to provide C23 (8.75 g, crude) as a white solid. The solid was used directly in the next step without further purification. (LCMS) m/z (M+H)⁺=310.0. ¹H NMR (400 MHz, (CD₃)₂SO) δ (ppm): 12.65 (br s, 1H), 4.85 (dd, 1H), 4.21-3.84 (m, 4H), 3.22-2.98 (m, 2H), 1.41 (br s, 9H), 1.35 (t, 3H), 1.16-1.08 (m, 3H). The chiral purity was determined using Chiral HPLC analytical method on Chiralcel OD-RH 150 mm×4.6 mm×5 μm column; Mobile phase A: H₂O (1.5 mL TFA)/Mobile phase B: ACN (1.5 mL TFA); gradient: 10-80% of Mobile phase B over 8 min then 10% of Mobile phase B in 1 min then a hold at 10% of Mobile phase B for 6 min, flow rate: 0.8 mL/min; column temperature: 30 °C to provide C23: peak 1 (retention time: 9.514 min, 100% ee). [α]³⁴_{D}=−95.69 (c=0.4 (g/L) in ACN).

### Step 2. Preparation of (S)-1-ethyl-7-methyl-4,5,6,7-tetrahydro-1H-pyrazolo[4,3-c]pyridine-3-carboxylic acid hydrochloride (P10)

At 0 °C, to C23 (8.00 g, 25.9 mmol) was added 2M HCl in dioxane (160 mL). The reaction mixture was stirred at room temperature for 4 h then was concentrated in vacuo to provide a yellow solid. The solid was dissolved in 2M HCl in dioxane (40 mL) and stirred at room temperature for 2 h then was concentrated in vacuo to provide P10 (7.10 g, crude) as a yellow solid. The solid was used directly in the next step without further purification. (LCMS) m/z (M+H)⁺=210.2. ¹H NMR (400 MHz, (CD₃)₂SO) δ (ppm): 12.97 (br s, 1H), 10.05 (s, 1H), 9.49 (s, 1H), 4.23-4.03 (m, 4H), 3.41-3.30 (m, 2H), 3.26-3.18 (m, 1H), 1.39-1.32 (m, 6H). The chiral purity was determined using SFC analytical method on Chiralcel OX-3 100 mm×4.6 mm, 3 μm column; Mobile phase A: CO₂/Mobile phase B: MeOH with 0.05% of DEA; Isocratic: 40% of Mobile phase B; backpressure: 100 bar; flow rate: 2.8 mL/min; column temperature: 40 °C to provide P10: peak 1 (retention time: 1.631 min, 100% ee). [α]³³_{D}=−1.584 (c=1.5 (g/L) in MeOH).

### Preparation 11

### 3-(4-(Aminomethyl)phenyl)-1,1-dimethylurea hydrochloride (P11)

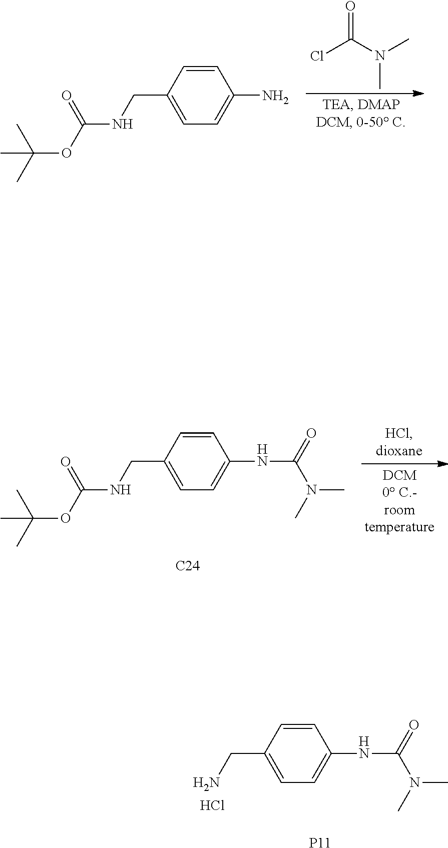

### Step 1. Preparation of tert-butyl (4-(3,3-dimethylureido)benzyl)carbamate (C24)

At 0 °C under nitrogen gas, to a solution of tert-butyl (4-aminobenzyl)carbamate (CAS: 94838-55-8; 15.0 g, 67.5 mmol) in DCM (150 mL), TEA (20.5 g, 202 mmol) and DMAP (0.824 g, 6.75 mmol) was added dimethylcarbamic chloride (14.5 g, 135 mmol) dropwise. The reaction mixture was stirred at 50 °C for 16 h then was diluted with MeOH (150 mL) and concentrated in vacuo to give a residue. The residue was suspended in H₂O (120 mL) then rapidly stirred at room temperature for 1 h. The suspension was filtered then the filter cake was collected and triturated with MeOH: EtOAc: MTBE (5 mL: 25 mL: 250 mL) at room temperature for 1 h. The suspension was filtered, and the filter cake was collected then concentrated in vacuo to provide C24 (14.6 g, 73.8% yield) as a white solid. (LC/MS) m/z (M+H)⁺=294.1. ¹H NMR (400 MHz, (CD₃)₂SO) δ (ppm): 8.21 (s, 1H), 7.37 (d, 2H), 7.30 (t, 1H), 7.08 (d, 2H), 4.03 (d, 2H), 2.91 (s, 6H), 1.39 (s, 9H)

### Step 2. Preparation of 3-(4-(aminomethyl)phenyl)-1,1-dimethylurea hydrochloride (P11)

At 0 °C, to a stirred suspension of C24 (23 g, 71 mmol) in DCM (130 mL) was added 2M HCl in dioxane (270 mL) dropwise. The reaction mixture was stirred at room temperature for 3 h then was concentrated in vacuo to provide P11 (15 g, 83.4%) as a yellow solid. The solid was used directly in the next step without additional purification. (LC/MS) m/z (2M+H)⁺=387.1. ¹H NMR (400 MHz, (CD₃)₂SO) δ (ppm): 8.40 (s, 1H), 8.25 (br s, 2H), 7.53-7.47 (m, 2H), 7.35-7.29 (m, 2H), 3.91 (q, 2H), 2.92 (s, 6H).

### Preparation 12

### N-(4-(Aminomethyl)phenyl)-4-methylpiperazine-1-carboxamide hydrochloride (P12)

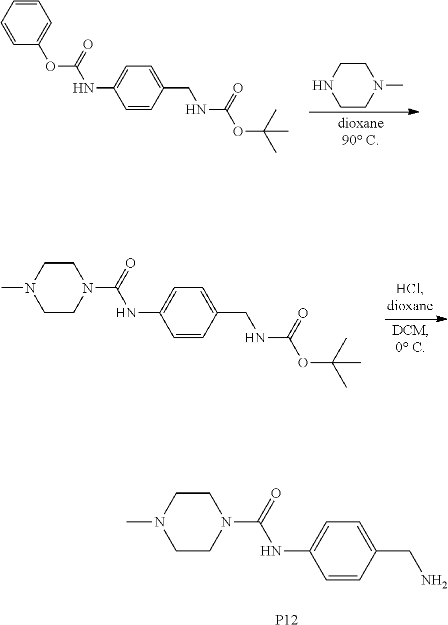

### Step 1. Preparation of tert-butyl (4-(4-methylpiperazine-1-carboxamido)benzyl)carbamate (C25)

To a solution of phenyl (4-(((tert-butoxycarbonyl)amino)methyl)phenyl)carbamate (CAS: 1632297-04-2; 8.0 g, 23 mmol) in dioxane (120 mL) was added 1-methylpiperazine (CAS: 109-01-3; 2.8 g, 28 mmol). The reaction mixture was stirred at 90 °C overnight. The suspension was diluted with EtOAc (100 mL) and washed with 1 M NaOH (2×50 mL) then brine (50 mL). The organic layer was dried with Na₂SO₄ and concentrated in vacuo. The residue was triturated with PE (2×50 mL) over 20 min then the suspension was filtered. The filter cake was collected to provide C25 (7.7 g, crude) as a brown solid. The solid was used directly in the next step without further purification. (LC/MS) m/z (M+H)⁺=349.2. ¹H NMR (400 MHz, (CD₃)₂SO) δ (ppm): 8.44 (s, 1H), 7.35 (d, 2H), 7.28 (t, 1H), 7.07 (d, 2H), 4.05-3.95 (m, 2H), 3.40 (t, 4H), 2.29 (t, 4H), 2.18 (s, 3H), 1.40-1.32 (m, 9H).

### Step 2. Preparation of N-(4-(aminomethyl)phenyl)-4-methylpiperazine-1-carboxamide hydrochloride (P12)

To a stirred solution of C25 (7.7 g, 22 mmol) in DCM (20 mL) was added HCl in dioxane (0.050 g, 1.4 mmol). The reaction mixture was stirred at 0 °C for 4 h then concentrated in vacuo and lyophilized to provide P12 (6.9 g, crude) as a white solid. The solid was used directly in the next step without further purification. (LC/MS) m/z (M+H)⁺=249.1. ¹H NMR (400 MHz, (CD₃)₂SO) δ (ppm): 11.27 (br s, 1H), 9.08 (s, 1H), 8.36 (br s, 3H), 7.53-7.47 (m, 2H), 7.34-7.29 (m, 2H), 4.25 (d, 2H), 3.87 (q, 2H), 3.31-3.18 (m, 2H), 3.04-2.89 (m, 2H), 2.72 (s, 3H).

### Preparation 13

### Ethyl (S)-1-isopropyl-7-methyl-4,5,6,7-tetrahydro-1H-pyrazolo[4,3-c]pyridine-3-carboxylate hydrochloride (P13)

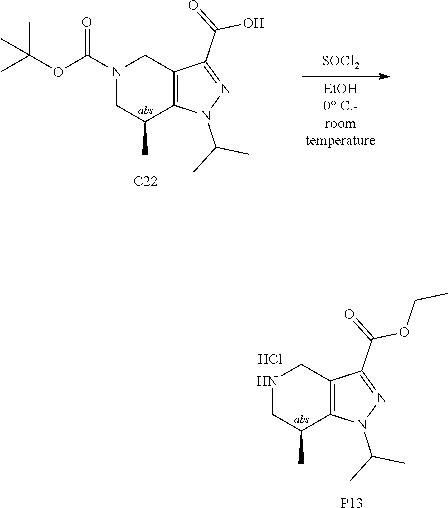

### Step 1. Preparation of ethyl (S)-1-isopropyl-7-methyl-4,5,6,7-tetrahydro-1H-pyrazolo[4,3-c]pyridine-3-carboxylate hydrochloride (P13)

To a stirred solution of C22 (0.500 g, 1.55 mmol) in EtOH (3 mL) was added SOCl₂ (1.29 g, 10.8 mmol) dropwise at 0 °C. The reaction mixture was stirred at room temperature for 66 h then concentrated in vacuo to provide P13 (0.426 g, 95.7% yield) as a light-yellow solid. (LC/MS) m/z (M+H)⁺=252.2.

### Preparation 14

### Ethyl 1-(1-methylcyclopropyl)-4,5,6,7-tetrahydro-1H-pyrazolo[4,3-c]pyridine-3-carboxylate (P14)

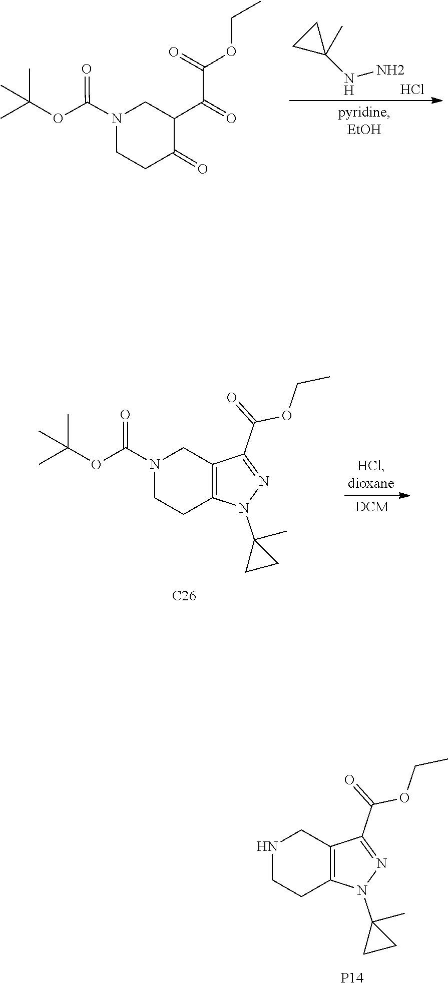

### Step 1. Preparation of 5-(tert-butyl) 3-ethyl 1-(1-methylcyclopropyl)-1,4,6,7-tetrahydro-5H-pyrazolo[4,3-c]pyridine-3,5-dicarboxylate (C26)

The same procedure was followed from Preparation 2, step 1 with tert-butyl 3-(2-ethoxy-2-oxoacetyl)-4-oxopiperidine-1-carboxylate (CAS: 518990-24-4; 0.577 g, 1.93 mmol) and (1-methylcyclopropyl)hydrazine hydrochloride (CAS: 2737246-42-1, 0.166 g, 1.93 mmol). The residue was purified by column chromatography (silica gel, 0-30% EtOAc: PE) to provide C26 (0.233 g, 34.6% yield) as a light-brown oil. (LC/MS) m/z (M+H)⁺=350.1. ¹H NMR (400 MHz, CDCl₃) δ (ppm): 4.58 (s, 2H), 4.38 (q, 2H), 3.74-3.66 (m, 2H), 2.83-2.74 (m, 2H), 1.52 (s, 3H), 1.48 (s, 9H), 1.37 (t, 3H), 1.28-1.23 (m, 2H), 0.97-0.91 (m, 2H).

### Step 2. Preparation of ethyl 1-(1-methylcyclopropyl)-4,5,6,7-tetrahydro-1H-pyrazolo[4,3-c]pyridine-3-carboxylate (P14)

The same procedure was followed from Preparation 5, step 2 with C26 (0.233 g, 0.667 mmol) to provide P14 (0.191 g, crude) as a light-yellow solid, which was used directly in the next step without further purification. (LC/MS) m/z (M+H)⁺=250.1. ¹H NMR (400 MHz, CD₃OD) δ (ppm): 4.39-4.33 (m, 2H), 3.66 (s, 2H), 3.56 (t, 2H), 3.19 (t, 2H), 1.55 (s, 3H), 1.38 (t, 3H), 1.31-1.26 (m, 2H), 1.09-1.04 (m, 2H).

### Preparation 15

### Ethyl 4,5,6,7-tetrahydrothieno[3,2-c]pyridine-3-carboxylate hydrochloride (P15)

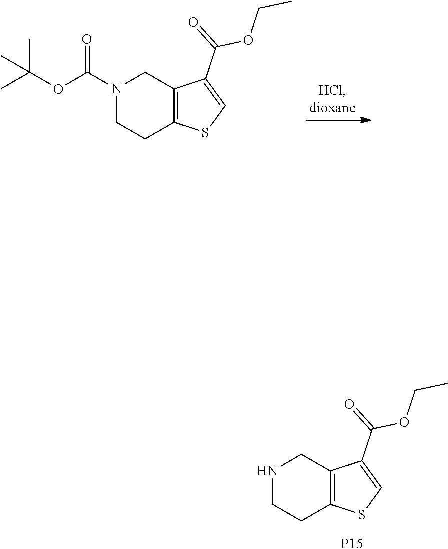

### Step 1. Preparation of ethyl 4,5,6,7-tetrahydrothieno[3,2-c]pyridine-3-carboxylate hydrochloride (P15)

The same reaction was conducted in two batches then combined. To 5-(tert-butyl) 3-ethyl 6,7-dihydrothieno[3,2-c]pyridine-3,5(4H)-dicarboxylate (CAS: 1363381-39-8; 50.0 mg, 0.161 mmol) was added HCl in dioxane (0.234 g, 6.42 mmol) to form the first batch. The solution of the first batch was stirred at room temperature for 2 h then concentrated in vacuo to provide a white solid.

A second batch of the same reaction was conducted with 5-(tert-butyl) 3-ethyl 6,7-dihydrothieno[3,2-c]pyridine-3,5(4H)-dicarboxylate (CAS: 1363381-39-8; 0.450 g, 1.45 mmol). The solids from the two batches were combined to provide P15 (0.339 g, crude) as a white solid. The solid was used directly in the next step without further purification. (LC/MS) m/z (M+H)⁺=212.0.

### Preparation 16

### Ethyl 1-methyl-5-(((trifluoromethyl)sulfonyl)oxy)-6,7-dihydro-1H-indazole-3-carboxylate (P16)

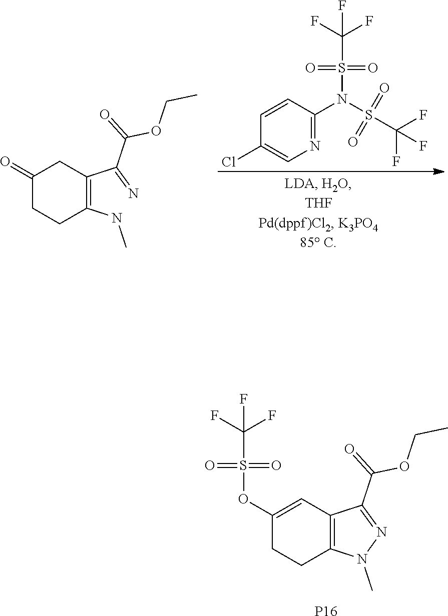

### Step 1. Preparation of ethyl 1-methyl-5-(((trifluoromethyl)sulfonyl)oxy)-6,7-dihydro-1H-indazole-3-carboxylate (P16)

To a suspension of ethyl 1-methyl-5-oxo-4,5,6,7-tetrahydro-1H-indazole-3-carboxylate (CAS: 2090404-84-3; 0.500 g, 1.41 mmol) and N-(5-chloropyridin-2-yl)-1,1,1-trifluoro-N-((trifluoromethyl)sulfonyl)methanesulfonamide (CAS: 145100-51-2; 0.409 g, 2.12 mmol) in H₂O (1 mL) and THF (10 mL) was added Pd(dppf)Cl₂ (0.103 g, 0.141 mmol) and K₃PO₄ (0.899 g, 4.23 mmol). The reaction mixture was degassed with nitrogen gas and stirred at 85 °C for 16 h then concentrated in vacuo. The residue was purified by column chromatography (silica gel, 0-38% THF: PE) to provide P16 (0.470 g, 94.2% yield) as a pink solid. (LC/MS) m/z (M+H)⁺=355.0

### Preparation 17

### Ethyl 1-(2,2,2-trifluoroethyl)-4,5,6,7-tetrahydro-1H-pyrazolo[4,3-c]pyridine-3-carboxylate hydrochloride (P17)

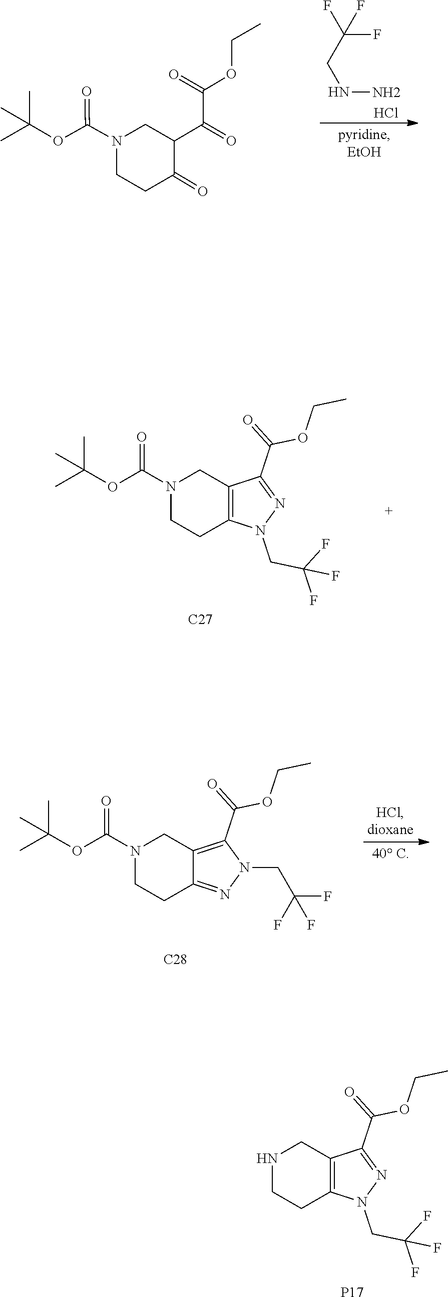

### Step 1. Preparation of 5-(tert-butyl) 3-ethyl 1-(2,2,2-trifluoroethyl)-1,4,6,7-tetrahydro-5H-pyrazolo[4,3-c]pyridine-3,5-dicarboxylate (C27) and 2-((3-(ethoxycarbonyl)-2-(2,2,2-trifluoroethyl)-4,5,6,7-tetrahydro-2H-pyrazolo[4,3-c]pyridine-5-carbonyl)oxy)-2-methylpropan-1-ylium (C28)

To a suspension of (2,2,2-trifluoroethyl)hydrazine hydrochloride (CAS: 1081515-82-3; 0.226 g, 1.50 mmol) in EtOH (5 mL) and pyridine (0.476 g, 6.01 mmol) was added tert-butyl 3-(2-ethoxy-2-oxoacetyl)-4-oxopiperidine-1-carboxylate (CAS: 518990-24-4; 0.450 g, 1.50 mol). The reaction mixture was stirred at room temperature for 17 h then was checked by LCMS which showed the major isomer of C27 [LC/MS) m/z (M-tert-butyl group)⁺=321.9 at 0.87 min retention time] and the minor isomer of C28 [(LC/MS) m/z (M+H)⁺=378.2 at 0.94 min retention time]. The suspension was concentrated in vacuo then dissolved in DCM and purified by column chromatography (silica gel, 0-40% EtOAc: heptane) to provide the major isomer of C27 (eluted at 30-40% EtOAc: heptane) (0.344 g, 60.7% yield) as a yellow oil. (LC/MS) m/z (M-tert-butyl group)+=322.2. ¹H NMR (600 MHz, CDCl₃) δ (ppm): 4.69 (q, 2H), 4.62 (br s, 2H), 4.41 (q, 2H), 3.77-3.70 (m, 2H), 2.74-2.70 (m, 2H), 1.56-1.43 (m, 9H), 1.40 (t, 3H)

### Step 2. Preparation of ethyl 1-(2,2,2-trifluoroethyl)-4,5,6,7-tetrahydro-1H-pyrazolo[4,3-c]pyridine-3-carboxylate hydrochloride (P17)

To a solution of C27 (0.344 g, 0.912 mmol) in DCM (5.0 mL) was added 4M HCl in dioxane (1.4 mL). The reaction solution was stirred at 40 °C for 24 h then concentrated in vacuo to provide P17 (0.328 g, crude) as a white solid. (LC/MS) m/z (M+H)⁺=278.2. ¹H NMR (600 MHz, (CD₃)₂SO) δ (ppm): 9.20 (br s, 1H), 5.31 (q, 2H), 4.32-4.22 (m, 4H), 3.39 (t, 2H), 2.99 (t, 2H), 1.29-1.26 (m, 3H).

### Preparation 18

### rac-Ethyl (R)-1,6-dimethyl-4,5,6,7-tetrahydro-1H-pyrazolo[4,3-c]pyridine-3-carboxylate (P18)

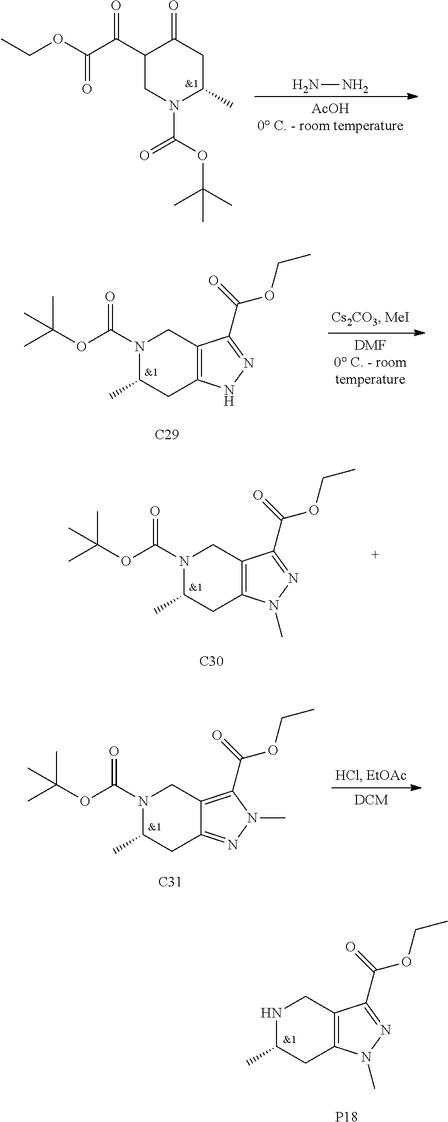

### Step 1. Preparation of rac-5-(tert-butyl) 3-ethyl (R)-6-methyl-1,4,6,7-tetrahydro-5H-pyrazolo[4,3-c]pyridine-3,5-dicarboxylate (C29)

At 0 °C, to a suspension of rac-tert-butyl(2R)-5-(2-ethoxy-2-oxoacetyl)-2-methyl-4-oxopiperidine-1-carboxylate (CAS: 2386748-67-8; 24 g, 77 mmol) in AcOH (120 mL) was added hydrazine monohydrate (CAS: 7803-57-8; 14 g, 0.18 mol) dropwise via addition funnel. The reaction was stirred at room temperature for 1 h then concentrated in vacuo. The residue was diluted in EtOAc (200 mL) then washed with aqueous NaHCO₃. The organic layer was dried over Na₂SO₄, filtered, and concentrated in vacuo to provide C29 (23 g, crude) as an orange viscous liquid. The liquid was used directly in the next step without further purification. (LC/MS) m/z (M+H)⁺=310.2. ¹H NMR (400 MHz, CDCl₃) δ (ppm): 10.21 (s, 1H), 5.06 (d, 1H), 4.85 (s, 1H), 4.44-4.28 (m, 2H), 4.15-4.10 (m, 1H), 2.96 (dd, 1H), 2.67-2.58 (m, 1H), 1.48-1.46 (m, 9H), 1.44-1.32 (m, 3H), 1.10 (d, 3H).

### Step 2. Preparation of rac-5-(tert-butyl) 3-ethyl (R)-1,6-dimethyl-1,4,6,7-tetrahydro-5H-pyrazolo[4,3-c]pyridine-3,5-dicarboxylate (C30) and rac-(R)-2-((3-(ethoxycarbonyl)-2,6-dimethyl-4,5,6,7-tetrahydro-2H-pyrazolo[4,3-c]pyridine-5-carbonyl)oxy)-2-methylpropan-1-ylium (C31)

At 0 °C, to a solution C29 (23 g, 74 mmol) in DMF (200 mL) was added Cs₂CO₃ (27 g, 82 mmol) and Mel (14 mL) dropwise. The reaction mixture was stirred at room temperature for 20 h then concentrated in vacuo. The residue was dissolved in MTBE (200 mL) then wash with H₂O (200 mL). The organic layer was dried over Na₂SO₄, filtered, and concentrated in vacuo to form an orange oil. The residue was purified by column chromatography (silica gel, 0-100% isopropyl acetate: heptane) to provide C30 (3.0 g, 25% yield) and C31 (1.6 g, 14% yield).

C30: (LC/MS) m/z (M+H)⁺=324.2. ¹H NMR (400 MHz, CDCl₃) δ (ppm): 5.12-4.92 (m, 1H), 4.87-4.72 (m, 1H), 4.41-4.23 (m, 2H), 4.11 (s, 3H), 4.10-4.02 (m, 1H), 2.90 (dd, 1H), 2.59-2.48 (m, 1H), 1.48-1.44 (m, 9H), 1.39-1.34 (m, 3H), 1.09 (d, 3H).

C31: (LC/MS) m/z (M+H)⁺=324.2. ¹H NMR (400 MHz, CDCl₃) δ (ppm): 5.08-4.85 (m, 2H), 4.44-4.30 (m, 2H), 4.08 (d, 1H), 3.80 (s, 3H), 2.94-2.84 (m, 1H), 2.44-2.38 (m, 1H), 1.46 (s, 9H), 1.37 (t, 3H), 1.20 (d, 3H).

### Step 3. Preparation of rac-ethyl (R)-1,6-dimethyl-4,5,6,7-tetrahydro-1H-pyrazolo[4,3-c]pyridine-3-carboxylate (P18)

To a solution of C30 (0.460 g, 1.42 mmol) in DCM (5 mL) was added 1 M HCl in EtOAc (5 mL). The reaction solution was stirred at room temperature for 6 h then concentrated in vacuo to provide P18 (0.325 g, crude) as a white foam, which was used directly in the next step without further purification.

### Preparation 19

### Ethyl 3-(1,1-difluoroethyl)-5,6,7,8-tetrahydroimidazo[1,5-a]pyrazine-1-carboxylate hydrochloride (P19)

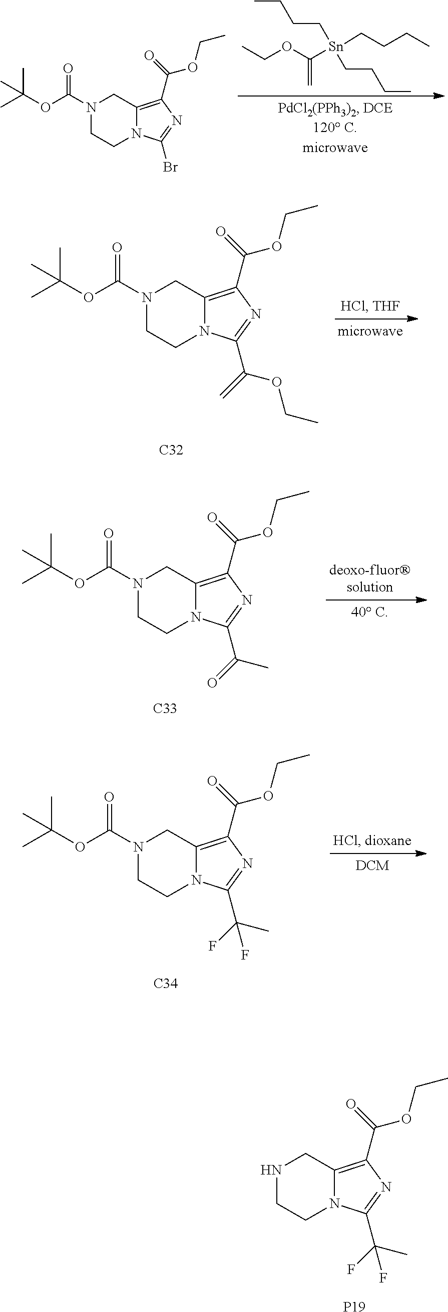

### Step 1. Preparation of 7-(tert-butyl) 1-ethyl 3-(1-ethoxyvinyl)-5,6-dihydroimidazo[1,5-a]pyrazine-1,7(8H)-dicarboxylate (C32)

To a solution of 7-(tert-butyl) 1-ethyl 3-bromo-5,6-dihydroimidazo[1,5-a]pyrazine-1,7(8H)-dicarboxylate (CAS: 2108354-93-2; 1.50 g, 4.01 mmol) in DCE (30 mL) was added tributyl(1-ethoxyvinyl)tin (CAS: 97674-02-7; 4.45 g, 12.3 mmol) and PdCl₂(PPh₃)₂(0.422 g, 0.601 mmol). The reaction mixture was stirred in a microwave at 120 °C for 30 min then was concentrated in vacuo. The residue was purified by column chromatography (silica gel; 0-27% THF: PE) to provide C32 (0.960 g, 65.5% yield) as an oil. (LC/MS) m/z (M+H)⁺=366.1. ¹H NMR (400 MHz, CDCl₃) δ (ppm): 5.06 (d, 1H), 4.90 (s, 2H), 4.42-4.33 (m, 2H), 4.18 (t, 2H), 3.90 (q, 2H), 3.79-3.71 (m, 3H), 1.50 (br s, 9H), 1.44-1.33 (m, 6H).

### Step 2. Preparation of 7-(tert-butyl) 1-ethyl 3-acetyl-5,6-dihydroimidazo[1,5-a]pyrazine-1,7(8H)-dicarboxylate (C33)

To a solution of C32 (0.660 g, 1.81 mmol) in THF (12 mL) was added 2M HCl (0.132 g, 3.61 mmol). The reaction mixture was stirred in the microwave for 16 h then the clear yellow solution was concentrated in vacuo. The residue was poured into saturated NaHCO₃ then extracted with EtOAc (3×20 mL). The combined organic layer was dried over MgSO₄, filtered, and concentrated in vacuo. The brown residue was purified by column chromatography (silica gel, 0-20% EtOAc: PE) to provide C33 (0.440 g, 81.1% yield) as a white solid. (LC/MS) m/z (M+H)⁺=338.3. ¹H NMR (400 MHz, CDCl₃) δ (ppm): 4.95 (s, 2H), 4.47 (t, 2H), 4.41 (q, 2H), 3.79 (t, 2H), 2.70 (s, 3H), 1.50 (s, 9H), 1.41 (t, 3H).

### Step 3. Preparation of 7-(tert-butyl) 1-ethyl 3-(1,1-difluoroethyl)-5,6-dihydroimidazo[1,5-a]pyrazine-1,7(8H)-dicarboxylate (C34)

A solution of C33 (0.435 g, 1.29 mmol) in Deoxo-Fluor™ solution (CAS: 202289-38-1; 10 mL) was stirred at 40 °C for 72 h. The reaction mixture was cooled to room temperature then extracted with DCM (2×20 mL). The combined organic layer was dried over MgSO₄, filtered, and concentrated in vacuo. The residue was purified by column chromatography (silica gel, 0-20% EtOAc: PE) to provide C34 (0.310 g, 66.9% yield) as a yellow gum. (LC/MS) m/z (M+H)⁺=360.1. ¹H NMR (400 MHz, CDCl₃) δ (ppm): 4.92 (s, 2H), 4.38 (q, 2H), 4.25 (t, 2H), 3.83 (t, 2H), 2.19 (t, 3H), 1.50 (s, 9H), 1.39 (t, 3H).

### Step 4. Preparation of ethyl 3-(1,1-difluoroethyl)-5,6,7,8-tetrahydroimidazo[1,5-a]pyrazine-1-carboxylate hydrochloride (P19)

To a solution of C34 (0.310 g, 0.863 mmol) in DCM (3 mL) was added 2M HCl in dioxane (6 mL). The reaction mixture was stirred at room temperature for 16 h then concentrated in vacuo and dried further by high vacuo to provide P19 (0.255 g, crude) as a gum. The gum was used directly in the next step without further purification.

### Preparation 20

### rac-Ethyl (R)-7-ethyl-4,5,6,7-tetrahydro-1H-pyrazolo[4,3-c]pyridine-3-carboxylate hydrochloride (P20)

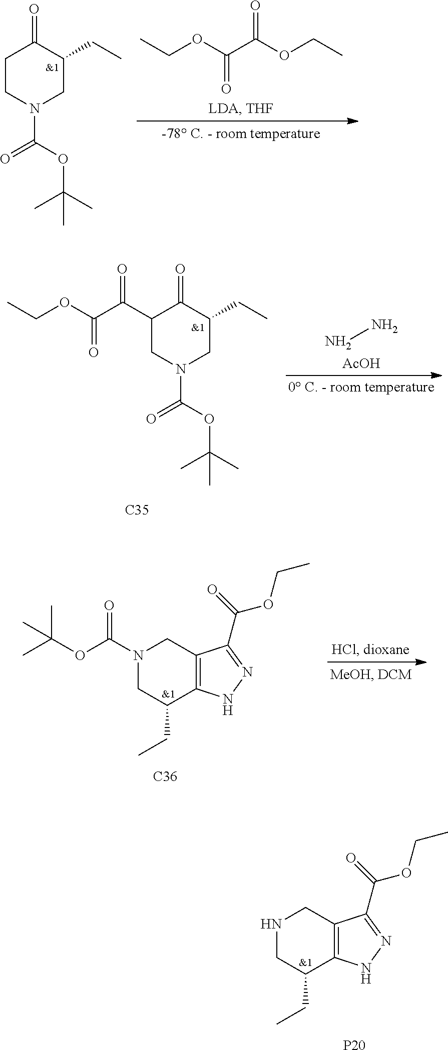

### Step 1. Preparation of rac-tert-butyl (5R)-3-(2-ethoxy-2-oxoacetyl)-5-ethyl-4-oxopiperidine-1-carboxylate (C35)

At −78 °C under nitrogen gas in a 250 mL three-neck round bottom flask, LDA (4.24 g, 39.6 mmol) was added to THF (25 mL). To the solution at −78 °C, tert-butyl 3-ethyl-4-oxopiperidine-1-carboxylate (CAS: 117565-57-8; 9.00 g, 39.6 mmol) in THF (60 mL) was added dropwise then stirred at −78 °C for 1 h. After the stir, a solution of diethyl oxalate (5.79 g, 39.6 mmol) in THF (20 mL) was added dropwise at −78 °C then stirred at −78 °C for 1 h. The reaction mixture warmed to room temperature then stirred for 16 h. The suspension was cooled to 0 °C then quenched with 1 M aqueous KHSO₄ (150 mL) and filtered. The filtrate was extracted with EtOAc (2×200 mL). The combined organic layer was dried over Na₂SO₄, filtered, and concentrated in vacuo to provide C35 (13.0 g, crude) as a yellow gum. The gum was used directly in the next step without further purification. (LC/MS) m/z (M-tert-butyl group)+=272.0. ¹H NMR (400 MHz, CDCl₃) δ (ppm): 15.42 (br s, 1H), 4.53-4.16 (m, 4H), 3.68-3.32 (m, 2H), 2.44-2.32 (m, 1H), 1.84-1.67 (m, 1H), 1.53-1.42 (m, 2H), 1.41-1.39 (m, 9H), 1.31 (t, 3H), 0.97 (t, 3H).

### Step 2. Preparation of rac-5-(tert-butyl) 3-ethyl (R)-7-ethyl-1,4,6,7-tetrahydro-5H-pyrazolo[4,3-c]pyridine-3,5-dicarboxylate (C36)

The same procedure was followed from Preparation 18, step 1 with C35 (6.00 g, 18.3 mmol) to provide C36 (8.89 g, crude) as a yellow gum, which was used directly in the next step without further purification. (LC/MS) m/z (M+H)⁺=324.1. ¹H NMR (400 MHz, CDCl₃) δ (ppm): 8.00 (br s, 1H), 4.93-4.44 (m, 2H), 4.37 (q, 2H), 3.83-3.68 (m, 1H), 3.57-3.41 (m, 1H), 2.88-2.71 (m, 1H), 1.84-1.70 (m, 1H), 1.62-1.51 (m, 2H), 1.50-1.48 (m, 9H), 1.38 (t, 3H), 1.05 (t, 3H).

### Step 3. Preparation of rac-ethyl (R)-7-ethyl-4,5,6,7-tetrahydro-1H-pyrazolo[4,3-c]pyridine-3-carboxylate hydrochloride (P20)

To a suspension of C36 (0.400 g, 1.24 mmol) in DCM (6 mL) and MeOH (2 mL) was added 2M HCl in dioxane (3 mL). The reaction mixture was stirred at room temperature for 2 h then an additional portion of 2M HCl in dioxane (3 mL) was added. The suspension was stirred for 16 h at room temperature then concentrated in vacuo to provide P20 (0.276 g, crude) as a yellow solid. The solid was used directly in the next step without further purification. ¹H NMR (400 MHz, (CD₃)₂SO) δ (ppm): 9.68 (br s, 2H), 4.37-4.11 (m, 4H), 3.52-3.44 (m, 1H), 3.14-3.06 (m, 1H), 3.05-2.94 (m, 1H), 1.99-1.85 (m, 1H), 1.67-1.53 (m, 1H), 1.29 (t, 3H), 0.94 (t, 3H).

### Preparation 21

### rac-(R)-7-Ethyl-1-methyl-4,5,6,7-tetrahydro-1H-pyrazolo[4,3-c]pyridine-3-carboxylic acid hydrochloride (P21)

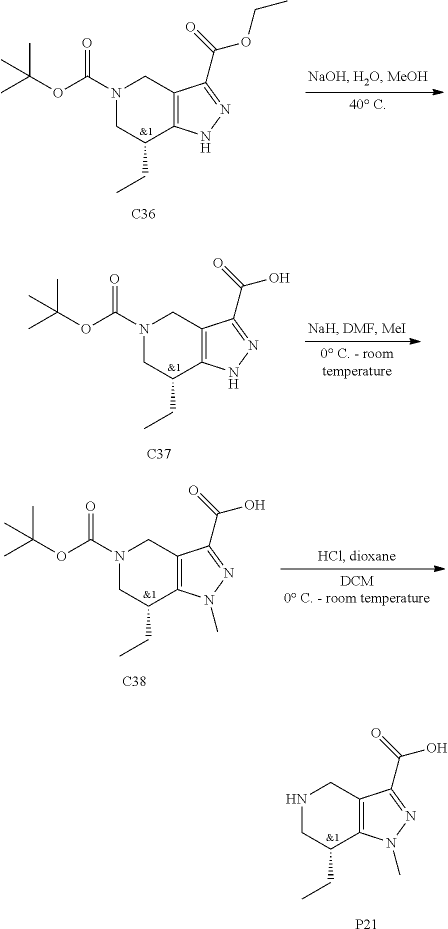

### Step 1. Preparation of rac-(R)-5-(tert-butoxycarbonyl)-7-ethyl-4,5,6,7-tetrahydro-1H-pyrazolo[4,3-c]pyridine-3-carboxylic acid (C37)

To a suspension of C36 (2.00 g, 6.18 mmol) in MeOH (6.6 mL) and H₂O (6.6 mL) was added NaOH (0.495 g, 12.4 mmol). The reaction mixture was stirred at 40 °C for 2 h then was diluted with H₂O (20 mL). The diluted reaction mixture was cooled to 0 °C then acidified with 1 M aqueous HCl to pH~3 and filtered. The filter cake was collected to provide C37 (1.60 g, crude) as a white solid. The solid was used directly in the next step without further purification. (LC/MS) m/z (M+H)⁺=296.1. ¹H NMR (400 MHz, (CD₃)₂SO) δ (ppm): 13.15 (br s, 1H), 4.73-4.24 (m, 2H), 3.74-3.41 (m, 2H), 2.78-2.67 (m, 1H), 1.70 (br s, 1H), 1.49-1.40 (m, 10H), 1.02 (t, 3H).

### Step 2. Preparation of rac-(R)-5-(tert-butoxycarbonyl)-7-ethyl-1-methyl-4,5,6,7-tetrahydro-1H-pyrazolo[4,3-c]pyridine-3-carboxylic acid (C38)

The same procedure was followed from Preparation 3, step 2 with C37 (1.00 g, 3.39 mmol) and Mel (0.577 g, 4.06 mmol) to provide C38 (0.850 g, crude) as a white solid, which was used directly in the next step without further purification. (LC/MS) m/z (M+H)⁺=310.1. ¹H NMR (400 MHz, (CD₃)₂SO) δ (ppm): 12.65 (br s, 1H), 5.01-4.75 (m, 1H), 4.34-3.88 (m, 2H), 3.79 (s, 3H), 3.07-2.67 (m, 2H), 1.63-1.50 (m, 1H), 1.43 (s, 9H), 1.36-1.23 (m, 1H), 1.08-0.95 (m, 3H).

### Step 3. Preparation of rac-(R)-7-ethyl-1-methyl-4,5,6,7-tetrahydro-1H-pyrazolo[4,3-c]pyridine-3-carboxylic acid hydrochloride (P21)

At 0 °C, to a solution of C38 (0.300 g, 0.970 mmol) in DCM (1 mL) was added HCl in dioxane (2 mL). The reaction mixture was warmed to room temperature and stirred for 16 h then concentrated in vacuo to provide P21 (0.236 g, crude) as a yellow solid. ¹H NMR (400 MHz, (CD₃)₂SO) δ (ppm): 12.97 (br s, 1H), 9.70 (br s, 1H), 9.13 (br s, 1H), 4.27-4.03 (m, 2H), 3.86 (s, 3H), 3.32-3.08 (m, 3H), 1.81-1.66 (m, 2H), 0.97 (t, 3H).

### Preparation 22

### 2-Chloro-5-(1-methyl-1H-imidazol-2-yl)pyridine (P22)

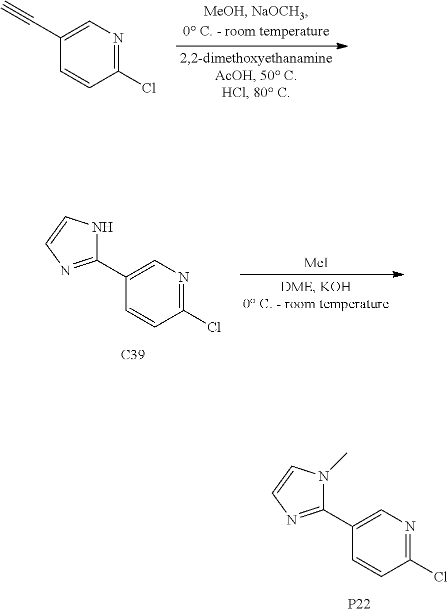

### Step 1. Preparation of 2-chloro-5-(1H-imidazol-2-yl)pyridine (C39)

At 0 °C, to a solution of 2-chloro-5-ethynylpyridine (CAS: 263012-63-1, 94.0 g, 680. mmol) in MeOH (1.5 L) was added NaOCH₃ (5.50 g, 102 mmol). The reaction was stirred at room temperature for 1 h before 2,2-dimethoxyethanamine (71.3 g, 678 mmol) and AcOH (81.5 g, 1.36 mol) were added. The reaction was stirred at 50 °C for 1 h then cooled to room temperature before 6M HCl (74.2 g, 2.04 mol) was added. The reaction was stirred at 80 °C for 5 h then concentrated in vacuo. The residue was extracted with EtOAc (2×500 mL). The aqueous layer was adjusted to pH=10 with saturated aqueous Na₂CO₃ solution which caused white solid precipitate to form. The suspension was filtered then the filter cake was washed with H₂O (3×100 mL). The filter cake was collected then lyophilized to provide C39 (88.0 g, 72.2% yield) as a white solid. The solid was used directly in the next step without additional purification. (LC/MS) m/z (M+H)⁺=180.0. ¹H NMR (400 MHz, (CD₃)₂SO) δ (ppm): 12.77 (br s, 1H), 8.94 (d, 1H), 8.31 (dd, 1H), 7.62 (d, 1H), 7.22 (s, 2H).

### Step 2. Preparation of 2-chloro-5-(1-methyl-1H-imidazol-2-yl)pyridine (P22)

To a solution of C39 (44.0 g, 204 mmol) in DME (440 mL) was added KOH (24.0 g, 428 mmol) at room temperature then stirred for 1 h and 40 min. The reaction mixture was cooled to 0 °C before Mel (57.8 g, 407 mmol) was added then stirred at 0 °C for 1 h. The white suspension was filtrated then concentrated in vacuo. The residue was washed with H₂O (300 mL) and stirred for 30 min. The reaction mixture was filtered, and the filter cake was concentrated in vacuo to provide P22 (34.8 g, 88.8% yield) as a white solid. The solid was used directly in the next step without additional purification. (LC/MS) m/z (M+H)⁺=194.0. ¹H NMR (400 MHz, (CD₃)₂SO) δ (ppm): 8.79-8.76 (m, 1H), 8.20 (dd, 1H), 7.68-7.64 (m, 1H), 7.37 (d, 1H), 7.08 (d, 1H), 3.83 (s, 3H).

### Preparation 23

### rac-Ethyl (R)-1-ethyl-7-methyl-4,5,6,7-tetrahydro-1H-pyrazolo[4,3-c]pyridine-3-carboxylate trifluoroacetate (P23)

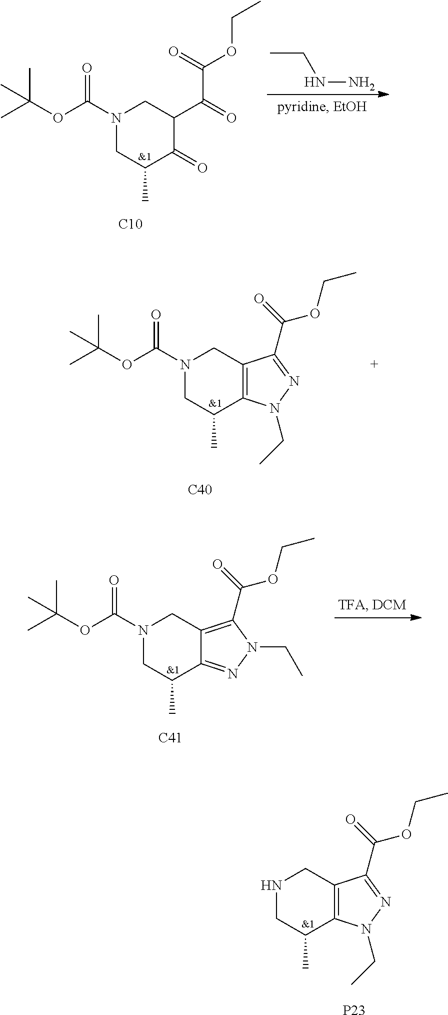

### Step 1. Preparation of rac-5-(tert-butyl) 3-ethyl (R)-1-ethyl-7-methyl-1,4,6,7-tetrahydro-5H-pyrazolo[4,3-c]pyridine-3,5-dicarboxylate (C40) and rac-5-(tert-butyl) 3-ethyl (R)-2-ethyl-7-methyl-2,4,6,7-tetrahydro-5H-pyrazolo[4,3-c]pyridine-3,5-dicarboxylate (C41)

A suspension of C10 (1.00 g, 3.19 mmol) and ethylhydrazine dihydrochloride (CAS: 49540-34-3; 0.425 g, 3.19 mmol) in EtOH (20 mL) and pyridine (1.11 g, 14.0 mmol) was stirred at room temperature for 16 h. The reaction mixture was concentrated in vacuo then purified by column chromatography (silica gel, 0-40% EtOAc: PE) to provide C41 (0.290 g, 26.9% yield) as a light-yellow solid and C40 (0.700 g, 65.0% yield) as a light-yellow gum.

C40: (LC/MS) m/z (M+H)⁺=338.2; ¹H NMR (400 MHz, CDCl₃) δ (ppm): 5.20-4.88 (m, 1H), 4.44-4.32 (m, 2H), 4.29-3.97 (m, 4H), 3.16 (d, 1H), 3.00-2.88 (m, 1H), 1.52-1.44 (m, 12H), 1.38 (t, 3H), 1.25 (d, 3H).

C41: (LC/MS) m/z (M+H)⁺=338.2; ¹H NMR (400 MHz, CDCl₃) δ (ppm): 4.75-4.46 (m, 4H), 4.33 (q, 2H), 3.96-3.64 (m, 1H), 3.36-3.08 (m, 1H), 3.00-2.91 (m, 1H), 1.50-1.44 (m, 9H), 1.39 (q, 6H), 1.26 (d, 3H).

### Step 2. Preparation of rac-ethyl (R)-1-ethyl-7-methyl-4,5,6,7-tetrahydro-1H-pyrazolo[4,3-c]pyridine-3-carboxylate trifluoroacetate (P23)

To a solution of C40 (0.250 g, 0.741 mmol) in DCM (4 mL) was added TFA (2 mL). The reaction mixture was stirred at room temperature for 2 h then concentrated in vacuo to provide P23 (0.260 g, crude) as a colorless gum, which was used directly in the next step without further purification.

### Preparation 24

### rac-Ethyl (4R,8S)-1-methyl-4,5,6,7,8,9-hexahydro-1H-4,8-epiminocycloocta[c]pyrazole-3-carboxylate (P24)

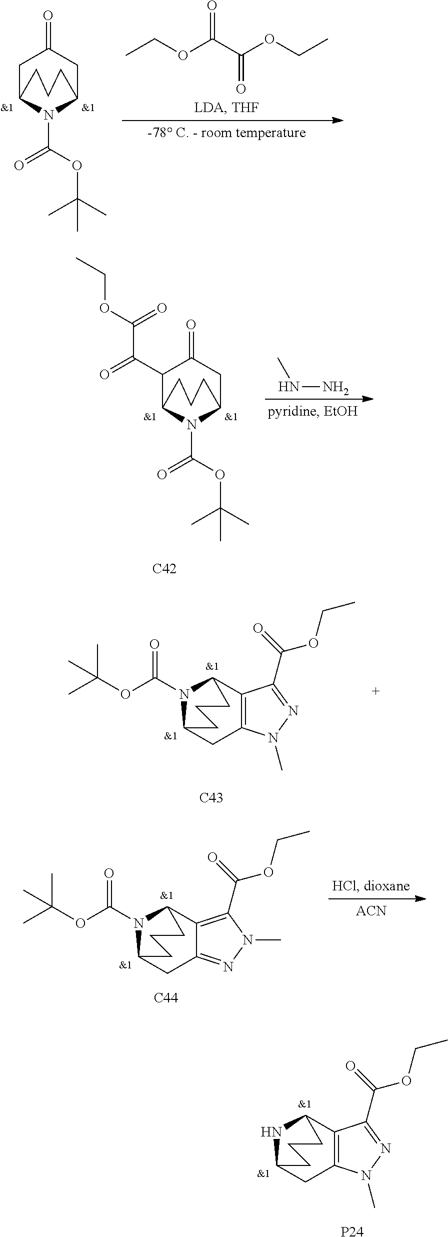

### Step 1. Preparation of rac-tert-butyl (1R,5S)-2-(2-ethoxy-2-oxoacetyl)-3-oxo-9-azabicyclo[3.3.1]nonane-9-carboxylate (C42)

At −78 °C, LDA (1.07 g, 10.0 mmol) was added to THF (50 mL). To the solution at −78 °C, tert-butyl (1R,5S)-3-oxo-9-azabicyclo[3.3.1]nonane-9-carboxylate (CAS: 512822-27-4; 2.00 g, 8.36 mmol) in THF (50 mL) was added dropwise and then stirred at −78 °C for 1 h. After the stir, a solution of diethyl oxalate (1.28 g, 8.78 mmol) in THF (20 mL) was added dropwise at −78 °C and then stirred at −78 °C for 30 min. The reaction mixture was warmed to room temperature, stirred for 1 h, and then concentrated in vacuo. The residue was purified by column chromatography (silica gel, 2-16% EtOAc: PE) to provide C42 (0.750 g, 26.4% yield) as a light yellow oil. (LC/MS) m/z (M+H)⁺=340.1 ¹H NMR (400 MHz, CDCl₃) δ (ppm): 5.49 (d, 1H), 4.77-4.48 (m, 2H), 4.36 (q, 2H), 2.98-2.81 (m, 1H), 2.44-2.31 (m, 1H), 1.86-1.72 (m, 3H), 1.69-1.58 (m, 3H), 1.50-1.42 (m, 9H), 1.39 (t, 3H).

### Step 2. Preparation of rac-10-(tert-butyl) 3-ethyl (4R,8S)-1-methyl-4,5,6,7,8,9-hexahydro-1H-4,8-epiminocycloocta[c]pyrazole-3,10-dicarboxylate (C43) and rac-10-(tert-butyl) 3-ethyl (4R,8S)-2-methyl-4,5,6,7,8,9-hexahydro-2H-4,8-epiminocycloocta[c]pyrazole-3,10-dicarboxylate (C44)

To a solution of C42 (0.650 g, 2.17 mmol) in EtOH (20 mL) was added methylhydrazine dihydrochloride (CAS: 55330-60-4; 0.179 g, 2.17 mmol) and pyridine (0.859 g, 10.9 mmol). The reaction mixture was stirred at room temperature for 16 h and then concentrated in vacuo. The residue was purified by column chromatography (silica gel, 12-100% EtOAc: PE) to provide C43 (0.390 g, 51.4% yield) as an oil and C44 (0.150 g, 19.8% yield).

C43: (LC/MS) m/z (M+H)⁺=350.1. ¹H NMR (400 MHz, CDCl₃) δ (ppm): 5.68-5.53 (m, 1H), 4.79-4.57 (m, 1H), 4.46-4.28 (m, 2H), 3.80 (s, 3H), 3.11-2.95 (m, 1H), 2.43 (d, 1H), 1.89-1.69 (m, 4H), 1.63-1.55 (m, 1H), 1.50-1.41 (m, 10H), 1.40-1.34 (m, 3H).

C44: (LC/MS) m/z (M+H)⁺=350.2. ¹H NMR (400 MHz, CDCl₃) δ (ppm): 5.68-5.48 (m, 1H), 4.78-4.53 (m, 1H), 4.45-4.19 (m, 2H), 4.15-4.07 (m, 3H), 3.19-3.00 (m, 1H), 2.63-2.52 (m, 1H), 1.90-1.58 (m, 5H), 1.44 (s, 10H), 1.38 (t, 3H).

### Step 3. Preparation of rac-ethyl (4R,8S)-1-methyl-4,5,6,7,8,9-hexahydro-1H-4,8-epiminocycloocta[c]pyrazole-3-carboxylate (P24)

To a solution of C43 (0.222 g, 0.636 mmol) in ACN (6 mL) was added HCl in dioxane (0.116 g, 3.18 mmol). The reaction mixture was stirred at room temperature for 16 h then concentrated in vacuo to provide P24 (0.150 g, crude) as a white solid. The solid was used directly in the next step without further purification.

### Preparation 25

### Ethyl 7,7-difluoro-1-methyl-4,5,6,7-tetrahydro-1H-pyrazolo[4,3-c]pyridine-3-carboxylate hydrochloride (P25)

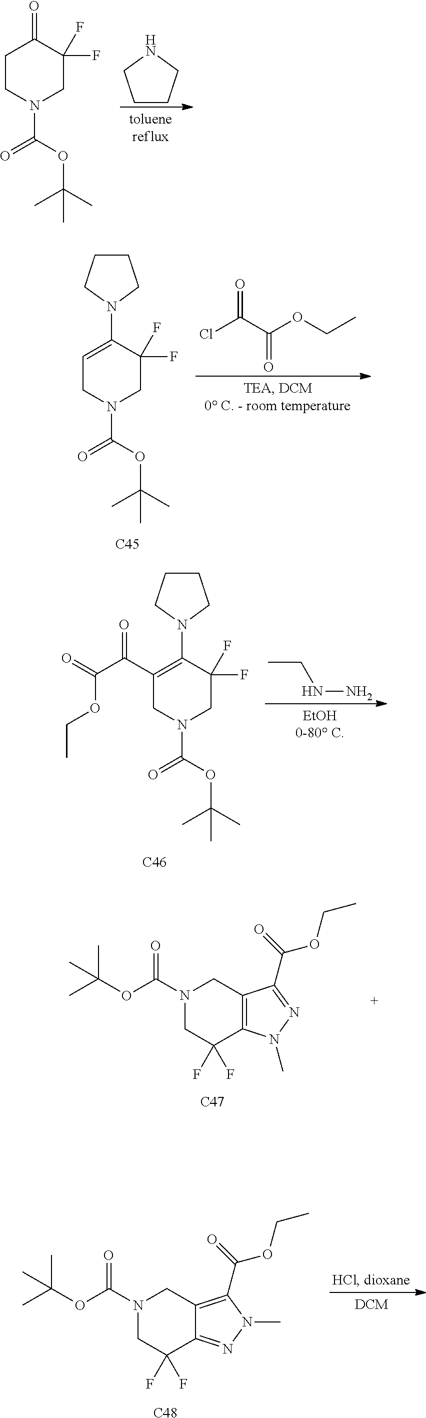

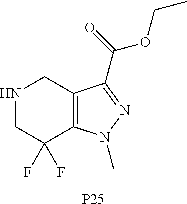

### Step 1. Preparation of tert-butyl 3,3-difluoro-4-(pyrrolidin-1-yl)-3,6-dihydropyridine-1(2H)-carboxylate (C45)

To a solution of tert-butyl 3,3-difluoro-4-oxopiperidine-1-carboxylate (CAS:1215071-17-2; 3.00 g, 11.8 mmol) in toluene (40 mL) was added pyrrolidine (0.927 g, 13.0 mmol). The reaction mixture was heated for 20 h using a Deans and Stark trap to remove the H₂O liberated during the reaction, then concentrated in vacuo to provide C45 (3.42 g, crude) as a brown solid. The solid was used directly in the next step without further purification. ¹H NMR (400 MHz, CDCl₃) δ (ppm): 4.66-4.46 (m, 1H), 4.14-4.05 (m, 2H), 3.90 (t, 2H), 3.20-3.13 (m, 4H), 1.97-1.88 (m, 4H), 1.53 (s, 9H).

### Step 2. Preparation of tert-butyl 5-(2-ethoxy-2-oxoacetyl)-3,3-difluoro-4-(pyrrolidin-1-yl)-3,6-dihydropyridine-1(2H)-carboxylate (C46)

A solution of C45 (3.42 g, 11.9 mmol) in DCM (50 mL) was degassed with nitrogen gas for 10 min. The reaction mixture was cooled to 0 °C before ethyl oxalyl chloride (1.94 g, 14.2 mmol) was added dropwise over 15 min. The temperature was maintained between (0-5) °C throughout the addition. Following the addition, TEA (1.80 g, 17.8 mmol) was added dropwise over 5 min. The reaction mixture was warmed to room temperature and stirred for 5 h, then diluted with H₂O (300 mL). The diluted reaction mixture was extracted with DCM (3×100 mL). The combined organic layer was dried with Na₂SO₄, filtered, and concentrated in vacuo. The residue was purified by column chromatography (silica gel, 0-25% EtOAc: PE) to provide C46 (2.98 g, 64.7% yield) as a yellow solid. (LC/MS) m/z (M+H)⁺=389.2. ¹H NMR (400 MHz, CDCl₃) δ (ppm): 4.41 (s, 2H), 4.30 (q, 2H), 3.89 (t, 2H), 3.49-3.41 (m, 4H), 2.00-1.94 (m, 4H), 1.48 (s, 9H), 1.35 (t, 3H).

### Step 3. Preparation of 5-(tert-butyl) 3-ethyl 7,7-difluoro-1-methyl-1,4,6,7-tetrahydro-5H-pyrazolo[4,3-c]pyridine-3,5-dicarboxylate (C47) and 2-((3-(ethoxycarbonyl)-7,7-difluoro-2-methyl-4,5,6,7-tetrahydro-2H-pyrazolo[4,3-c]pyridine-5-carbonyl)oxy)-2-methylpropan-1-ylium (C48)

The reaction was conducted in three batches and then combined for further purification. At 0 °C, to a solution of C46 (1.09 g, 2.81 mmol) in EtOH (10 mL) was added methylhydrazine dihydrochloride (CAS: 55330-60-4; 0.402 g, 3.38 mmol) dropwise over 5 min then the reaction was sealed and stirred at 80 °C for 2 h to form the first batch.

The second batch of the same reaction was conducted with C46 (0.500 g, 1.29 mmol), and the third batch was conducted with C46 (0.100 g, 0.257 mmol). The batches were combined and then diluted with H₂O (60 mL). The diluted reaction mixture was extracted with EtOAc (3×50 mL). The combined organic layer was dried over Na₂SO₄, filtered, and concentrated in vacuo. The residue was purified by column chromatography (silica gel, 0-15% EtOAc: PE) to provide C48 (0.756 g, 50.4% yield) as brown oil and an impure C47 as a residue. The residue, C47, was purified by reverse phase HPLC (C₁₈ 150 mm×40 mm×5 μm column, Mobile Phase A: H₂O (0.05% NH₄OH—NH₄HCO₃)/Mobile Phase B: ACN, 32 to 72% of Mobile Phase B over 9 min, 100% of Mobile Phase B hold 2 min, flow rate=60 mL/min) and lyophilized to provide C47 (0.416 g, 27.7% yield) as a colorless oil.

C47: (LC/MS) m/z (M-tert-butyl group)+=290.2; ¹H NMR (400 MHz, CDCl₃) δ (ppm): 4.69 (br s, 2H), 4.41 (q, 2H), 4.09-3.96 (m, 5H), 1.49 (s, 9H), 1.40 (t, 3H).

C48: (LC/MS) m/z (M-tert-butyl group)+=290.2; ¹H NMR (400 MHz, CDCl₃) δ (ppm): 4.73-4.63 (m, 2H), 4.37 (q, 2H), 4.23 (s, 3H), 4.06-3.95 (m, 2H), 1.49 (s, 9H), 1.40 (t, 3H).

### Step 4. Preparation of ethyl 7,7-difluoro-1-methyl-4,5,6,7-tetrahydro-1H-pyrazolo[4,3-c]pyridine-3-carboxylate hydrochloride (P25)

To a reaction mixture of C47 (0.416 g, 1.20 mmol) in DCM (5 mL) was added HCl in dioxane (1.76 g, 48.2 mmol) and then stirred at room temperature for 2.5 h. The reaction mixture was concentrated in vacuo to provide P25 (295 mg, crude) as a light-yellow solid. The solid was used directly in the next step without further purification. (LC/MS) m/z (M+H)⁺=246.1. ¹H NMR (400 MHz, CD₃OD) δ (ppm): 4.55 (t, 2H), 4.40 (q, 2H), 4.15-4.02 (m, 5H), 1.39 (t, 3H).

### Preparation 26

### rac-Ethyl (4R,8S)-1-methyl-1,4,5,7,8,9-hexahydro-4,8-epiminooxocino[5,4-c]pyrazole-3-carboxylate hydrochloride (P26)

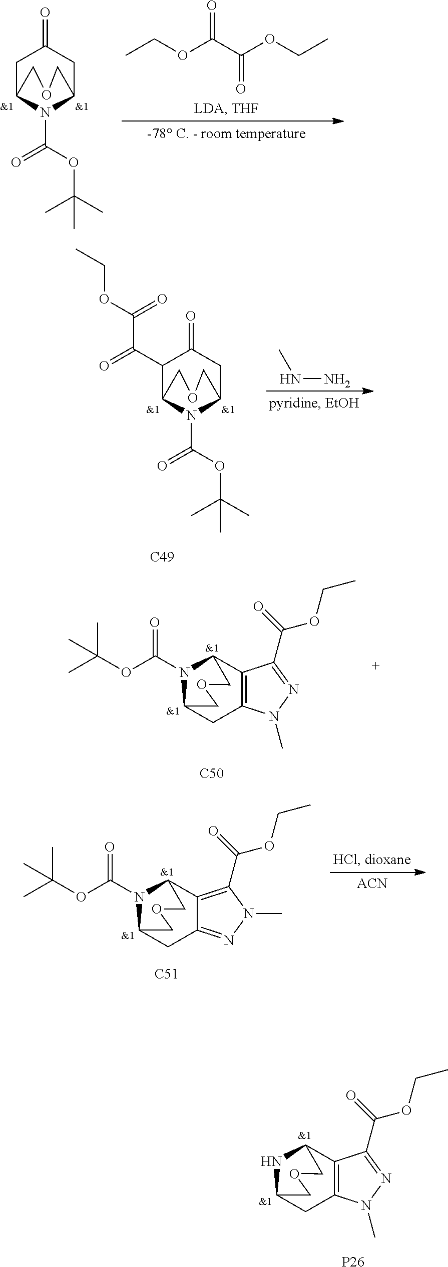

### Step 1. Preparation of rac-tert-butyl (1R,5S)-6-(2-ethoxy-2-oxoacetyl)-7-oxo-3-oxa-9-azabicyclo[3.3.1]nonane-9-carboxylate (C49)

The same procedure was followed from Preparation 20, step 1 with tert-butyl 7-oxo-3-oxa-9-azabicyclo[3.3.1]nonane-9-carboxylate (CAS: 280761-97-9; 1.80 g, 7.46 mmol) to provide C49 (2.50 g, crude) as an orange oil, which was used directly in the next step without further purification.

### Step 2. Preparation of rac-10-(tert-butyl) 3-ethyl (4R,8S)-1-methyl-1,4,5,7,8,9-hexahydro-4,8-epiminooxocino[5,4-c]pyrazole-3,10-dicarboxylate (C50) and rac-10-(tert-butyl) 3-ethyl (4R,8S)-2-methyl-2,4,5,7,8,9-hexahydro-4,8-epiminooxocino[5,4-c]pyrazole-3,10-dicarboxylate (C51)

The reaction was conducted in two batches and then combined. A suspension of C49 (2.50 g, 7.32 mmol) and methylhydrazine dihydrochloride (CAS: 55330-60-4; 0.871 g, 7.32 mmol) in EtOH (40 mL) and pyridine (2.55 g, 32.2 mmol) was stirred at room temperature for 3 h to form the first batch. The reaction mixture of the first batch was checked by LCMS, which showed the major isomer of C50 [LC/MS) m/z (M+H)⁺=352.3 at 0.827 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min column temperature: 50 °C)] and the minor isomer of C51 [LC/MS) m/z (M+H)⁺=352.3 at 0.880 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min column temperature: 50 °C)].

The second batch of the same reaction was conducted with C49 (0.200 g, 0.586 mmol). The two batches were combined, concentrated in vacuo, and purified by column chromatography (silica gel, 0-50% EtOAc: PE) to provide C50 (0.750 g, 27.0% yield) as a yellow solid. (LC/MS) m/z (M+H)⁺=352.3. ¹H NMR (400 MHz, CDCl₃) δ (ppm): 5.55-5.30 (m, 1H), 4.53-4.27 (m, 3H), 3.91-3.64 (m, 7H), 3.21-2.92 (m, 1H), 2.71-2.59 (m, 1H), 1.51-1.30 (m, 12H).

### Step 3. Preparation of rac-ethyl (4R,8S)-1-methyl-1,4,5,7,8,9-hexahydro-4,8-epiminooxocino[5,4-c]pyrazole-3-carboxylate hydrochloride (P26)

To a solution of C50 (0.750 g, 2.14 mmol) in DCM (5 mL) was added 2M HCl in dioxane (0.390 g, 10.7 mmol). The reaction mixture was stirred at room temperature for 2 h and then concentrated in vacuo. The residue was dissolved in DCM (5 mL) and then concentrated in vacuo (2×) to provide P26 (0.675 g, crude) as a yellow solid. The solid was used directly in the next step without further purification. ¹H NMR (400 MHz, CDCl₃) δ (ppm): 10.83 (s, 1H), 9.85 (s, 1H), 5.07 (s, 1H), 4.45-4.20 (m, 4H), 4.01-3.84 (m, 5H), 3.82-3.73 (m, 1H), 3.66-3.55 (m, 1H), 2.98-2.86 (m, 1H), 1.38 (t, 3H).

### Preparation 27

### Ethyl 1-propyl-4,5,6,7-tetrahydro-1H-pyrazolo[4,3-c]pyridine-3-carboxylate (P27)

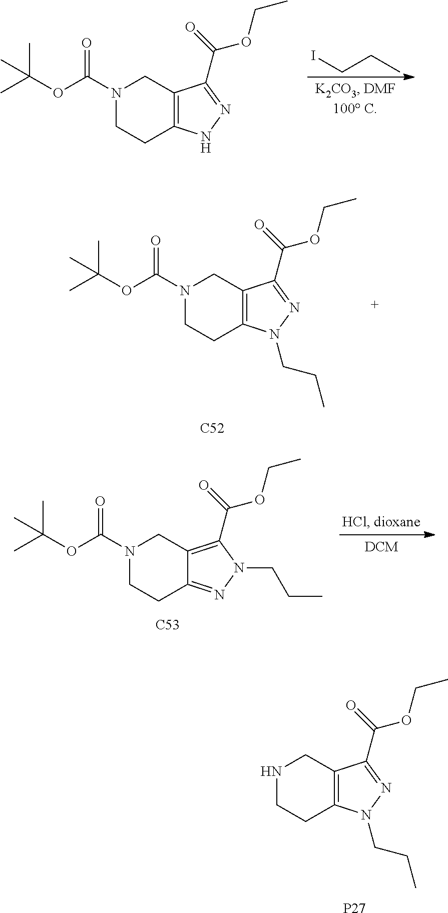

### Step 1. Preparation of 5-(tert-butyl) 3-ethyl 1-propyl-1,4,6,7-tetrahydro-5H-pyrazolo[4,3-c]pyridine-3,5-dicarboxylate (C52) and 2-((3-(ethoxycarbonyl)-2-propyl-4,5,6,7-tetrahydro-2H-pyrazolo[4,3-c]pyridine-5-carbonyl)oxy)-2-methylpropan-1-ylium (C53)

To a solution of 5-(tert-butyl) 3-ethyl 1,4,6,7-tetrahydro-5H-pyrazolo[4,3-c]pyridine-3,5-dicarboxylate (CAS: 518990-23-3; 0.81 g, 4.7 mmol) and 1-iodopropane (1.0 g, 3.4 mmol) in DMF (13 mL) was added K₂CO₃ (0.94 g, 6.7 mmol). The reaction mixture was stirred at 100 °C for 4 h, cooled to room temperature, and diluted with (1:1) EtOAc: H₂O (50 mL). The aqueous layer was extracted with EtOAc (3×25 mL). The combined organic layer was dried with Na₂SO₄, filtered, and concentrated in vacuo. The residue was purified by column chromatography (silica gel, 0-39% EtOAc: PE) to provide C53 (0.48 g, 42% yield) as a white solid and C52 (0.38 g, 33% yield) as a white solid.

C52: (LC/MS) m/z (M+H)⁺=338.3; ¹H NMR (400 MHz, CDCl₃) δ (ppm): 4.61 (s, 2H), 4.39 (q, 2H), 4.03 (t, 2H), 3.76-3.67 (m, 2H), 2.73-2.63 (m, 2H), 1.92-1.80 (m, 2H), 1.48 (s, 9H), 1.39 (t, 3H), 0.91 (t, 3H).

C53: (LC/MS) m/z (M+H)⁺=338.3; ¹H NMR (400 MHz, CDCl₃) δ (ppm): 4.65-4.56 (m, 2H), 4.49-4.43 (m, 2H), 4.33 (q, 2H), 3.68 (s, 2H), 2.74 (s, 2H), 1.90-1.78 (m, 2H), 1.49 (s, 9H), 1.39 (t, 3H), 0.92 (t, 3H).

### Step 2. Preparation of ethyl 1-propyl-4,5,6,7-tetrahydro-1H-pyrazolo[4,3-c]pyridine-3-carboxylate (P27)

To a reaction mixture of C52 (0.370 g, 1.10 mmol) in DCM (3 mL) was added 2M HCl in dioxane (0.400 g, 11.0 mmol) and then stirred at room temperature for 1 h and 40 min. The reaction mixture was concentrated in vacuo to provide P27 (0.300 g, crude) as a white solid. The solid was used directly in the next step without further purification. ¹H NMR (400 MHz, (CD₃)₂SO) δ (ppm): 9.54 (s, 2H), 4.25 (q, 2H), 4.19 (s, 2H), 4.06 (t, 2H), 3.40-3.35 (m, 2H), 2.97 (t, 2H), 1.80-1.68 (m, 2H), 1.27 (t, 3H), 0.82 (t, 3H).

### Preparation 28

### Ethyl 1-(tetrahydro-2H-pyran-4-yl)-4,5,6,7-tetrahydro-1H-pyrazolo[4,3-c]pyridine-3-carboxylate hydrochloride (P28)

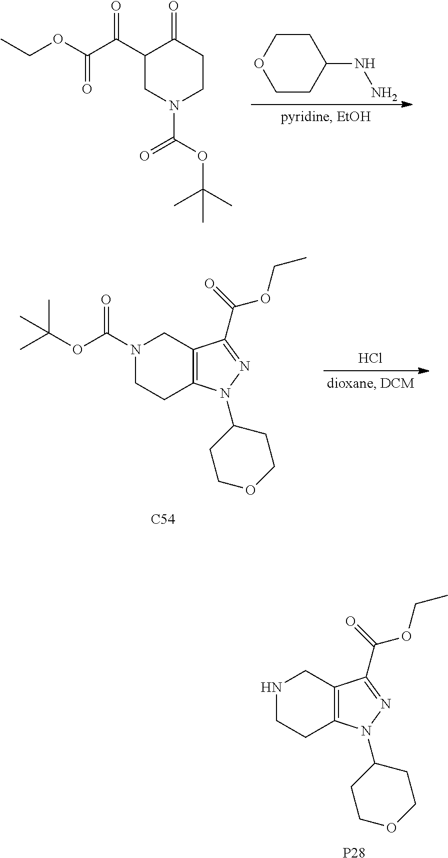

### Step 1. Preparation of 5-(tert-butyl) 3-ethyl 1-(tetrahydro-2H-pyran-4-yl)-1,4,6,7-tetrahydro-5H-pyrazolo[4,3-c]pyridine-3,5-dicarboxylate (C54)

The same procedure was followed from Preparation 2, step 1 with tert-butyl 3-(2-ethoxy-2-oxoacetyl)-4-oxopiperidine-1-carboxylate (CAS: 518990-24-4; 0.500 g, 1.67 mmol) and (tetrahydro-2H-pyran-4-yl)hydrazine hydrochloride (0.255 g, 1.67 mmol). The residue was purified by column chromatography (silica gel, 0-40% THF: PE) to provide C54 (0.492 g, 77.6% yield) as a yellow gum. (LC/MS) m/z (M+H)⁺=380.3. ¹H NMR (400 MHz, CDCl₃) δ (ppm): 4.60 (s, 2H), 4.38 (q, 2H), 4.31-4.21 (m, 1H), 4.15-4.07 (m, 2H), 3.77-3.68 (m, 2H), 3.54-3.44 (m, 2H), 2.73 (t, 2H), 2.41-2.25 (m, 2H), 1.89-1.80 (m, 2H), 1.51-1.40 (m, 9H), 1.38 (t, 3H).

### Step 2. Preparation of ethyl 1-(tetrahydro-2H-pyran-4-yl)-4,5,6,7-tetrahydro-1H-pyrazolo[4,3-c]pyridine-3-carboxylate hydrochloride (P28)

To a solution of C54 (0.470 g, 1.24 mmol) in DCM (10 mL) was added 2M HCl in dioxane (0.973 g, 26.7 mmol). The reaction mixture was stirred at room temperature for 4 h then concentrated in vacuo to provide P28 (0.370 g, crude) as a yellow solid. The solid was used directly in the next step without further purification. (LC/MS) m/z (M+H)⁺=280.1. ¹H NMR (400 MHz, (CD₃)₂SO) δ (ppm): 9.37 (s, 1H), 4.56-4.45 (m, 1H), 4.33-4.19 (m, 4H), 4.03-3.93 (m, 2H), 3.52-3.41 (m, 4H), 3.04 (t, 2H), 2.07-1.92 (m, 2H), 1.86-1.77 (m, 2H), 1.29 (t, 3H). Preparation 29 rac-Ethyl (R)-1-(sec-butyl)-4,5,6,7-tetrahydro-1H-pyrazolo[4,3-c]pyridine-3-carboxylate hydrochloride (P29)

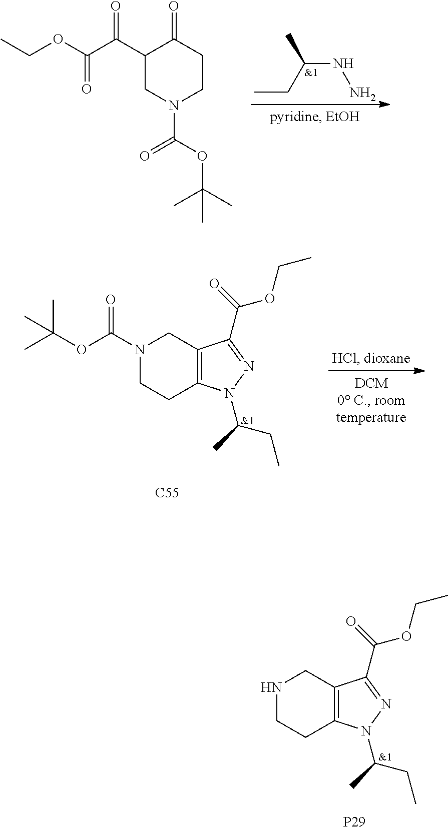

### Step 1. Preparation of rac-5-(tert-butyl) 3-ethyl (R)-1-(sec-butyl)-1,4,6,7-tetrahydro-5H-pyrazolo[4,3-c]pyridine-3,5-dicarboxylate (C55)

The same procedure was followed from Preparation 2, step 1 with tert-butyl 3-(2-ethoxy-2-oxoacetyl)-4-oxopiperidine-1-carboxylate (CAS: 518990-24-4; 0.400 g, 1.34 mmol) and sec-butylhydrazine dihydrochloride (CAS: 1177361-36-2, 0.215 g, 1.34 mmol). The residue was purified by column chromatography (silica gel, 50% EtOAc: PE) to provide C55 (0.382 g, 81.3% yield) as a gum. (LC/MS) m/z (M+H)⁺=352.1. ¹H NMR (400 MHz, (CD₃)₂SO) δ (ppm): 4.53-4.40 (m, 2H), 4.30-4.17 (m, 3H), 3.68-3.54 (m, 2H), 2.71 (q, 2H), 1.87-1.66 (m, 2H), 1.41 (s, 9H), 1.37 (d, 3H), 1.29 (t, 3H), 0.69 (t, 3H).

### Step 2. Preparation of rac-ethyl (R)-1-(sec-butyl)-4,5,6,7-tetrahydro-1H-pyrazolo[4,3-c]pyridine-3-carboxylate hydrochloride (P29)

At 0 °C, to a solution of C55 (0.382 g, 1.09 mmol) in DCM (2 mL) was added HCl in dioxane (4 mL). The reaction mixture was stirred at room temperature for 16 h then concentrated in vacuo to provide P29 (0.245 g, crude) as a solid. The solid was used directly in the next step without further purification.

### Preparation 30

### rac-Ethyl (4R,7S)-1-methyl-1,4,5,6,7,8-hexahydro-4,7-epiminocyclohepta[c]pyrazole-3-carboxylate (P30)

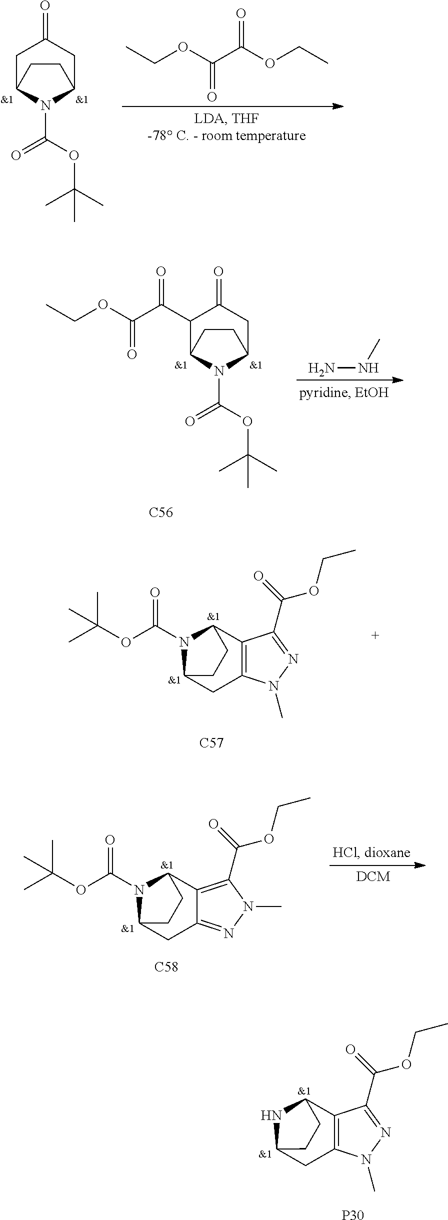

### Step 1. Preparation of rac-tert-butyl (1R,5S)-2-(2-ethoxy-2-oxoacetyl)-3-oxo-8-azabicyclo[3.2.1]octane-8-carboxylate (C56)

The same procedure was followed from Preparation 20, step 1, with tert-butyl (1R,5S)-8-oxo-3-azabicyclo[3.2.1]octane-3-carboxylate (CAS: 637301-19-0; 5.10 g, 22.6 mmol), with an additional purification added. The residue was purified by column chromatography (silica gel, 0-10% EtOAc: PE) to provide C56 (4.69 g, 63.7% yield) as a yellow oil.

### Step 2. Preparation of rac-9-(tert-butyl) 3-ethyl (4R,7S)-1-methyl-1,4,5,6,7,8-hexahydro-4,7-epiminocyclohepta[c]pyrazole-3,9-dicarboxylate (C57) and rac-9-(tert-butyl) 3-ethyl (4R,7S)-2-methyl-2,4,5,6,7,8-hexahydro-4,7-epiminocyclohepta[c]pyrazole-3,9-dicarboxylate (C58)

The same procedure was followed from Preparation 2, step 1 with C56 (4.69 g, 14.4 mmol) and methylhydrazine sulfate (CAS: 302-15-8, 2.08 g, 14.4 mmol). The reaction mixture was checked by LCMS, which showed the major isomer of C₅₋₇ [LC/MS) m/z (M+H)⁺=336.2 at 0.846 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min; column temperature: 50 °C)] and the minor isomer of C58 [LC/MS) m/z (M+H)⁺=336.2 at 0.904 min retention time (analytical LCMS conditions: Chromolith Flash Reverse Phase-18e 25-3 mm column; Mobile Phase A: 1.5 mL/4 L TFA in H₂O/Mobile Phase B: 0.75 mL/4 L TFA in ACN, gradient 5-95% of Mobile Phase B over 0.7 min then held at 95% of Mobile Phase B for 0.4 min; flow rate: 1.5 mL/min; column temperature: 50 °C)]. The purification was altered. The residue was purified by column chromatography (silica gel, 0-50% THF: PE) to provide C57 (1.40 g, 29.0% yield) as a light-yellow oil. ¹H NMR (400 MHz, CDCl₃) δ (ppm): 5.42-5.33 (m, 1H), 4.64-4.35 (m, 3H), 3.77 (s, 3H), 3.29-3.08 (m, 1H), 2.41-2.23 (m, 2H), 2.21-2.10 (m, 1H), 1.93-1.85 (m, 1H), 1.55-1.48 (m, 1H), 1.44-1.35 (m, 12H).

### Step 3. Preparation of ethyl 1-methyl-1,4,5,6,7,8-hexahydro-4,7-epiminocyclohepta[c]pyrazole-3-carboxylate (P30)

To a solution of C57 (0.240 g, 0.716 mmol) in DCM (1 mL), 2M HCl in dioxane (3 mL) was added. The reaction mixture was stirred at room temperature for 2.5 h and then concentrated in vacuo to provide P30 (0.168 g, crude) as a grey solid. The solid was used directly in the next step without further purification.

### Preparation 31

### Ethyl 1-(cyclopropylmethyl)-4,5,6,7-tetrahydro-1H-pyrazolo[4,3-c]pyridine-3-carboxylate (P31)

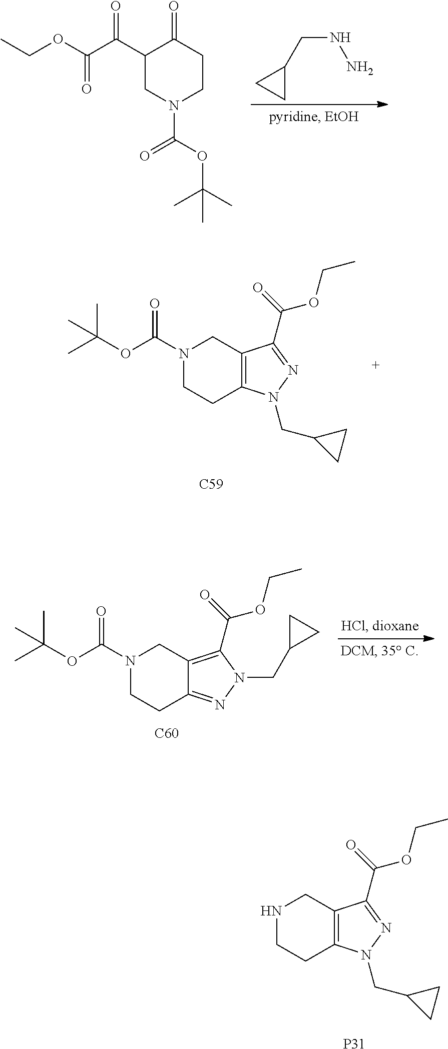

### Step 1. Preparation of 5-(tert-butyl) 3-ethyl 1-(cyclopropylmethyl)-1,4,6,7-tetrahydro-5H-pyrazolo[4,3-c]pyridine-3,5-dicarboxylate (C59) and 2-((2-(cyclopropylmethyl)-3-(ethoxycarbonyl)-4,5,6,7-tetrahydro-2H-pyrazolo[4,3-c]pyridine-5-carbonyl)oxy)-2-methylpropan-1-ylium (C60)

The same procedure was followed from Preparation 2, step 1 with tert-butyl 3-(2-ethoxy-2-oxoacetyl)-4-oxopiperidine-1-carboxylate (CAS: 518990-24-4; 2.00 g, 6.68 mmol) and (cyclopropylmethyl)hydrazine hydrochloride (CAS: 1181457-83-9, 0.655 g, 5.35 mmol). The residue was purified by column chromatography (silica gel, 0-60% EtOAc: PE) to provide an impure C60 (0.800 g) as a yellow oil and C59 (0.940 g, 40.3% yield) as a light-brown solid. C60 (0.800 g) was repurified by column chromatography (silica gel, 0-10% EtOAc: DCM) to provide C60 (0.450 g, 19.3% yield) as a grey gum.

C59: (LC/MS) m/z (M+H)⁺=350.1; ¹H NMR (400 MHz, (CD₃)₂SO) δ (ppm): 4.49 (s, 2H), 4.26 (q, 2H), 3.96 (d, 2H), 3.61 (t, 2H), 2.73 (t, 2H), 1.44-1.40 (m, 9H), 1.29 (t, 3H), 1.24-1.16 (m, 1H), 0.54-0.47 (m, 2H), 0.37-0.31 (m, 2H).

C60: (LC/MS) m/z (M+H)⁺=350.0; ¹H NMR (400 MHz, CDCl₃) δ (ppm): 4.62 (s, 2H), 4.41-4.30 (m, 4H), 3.76-3.61 (m, 2H), 2.81-2.71 (m, 2H), 1.50-1.46 (m, 9H), 1.42-1.30 (m, 4H), 0.54-0.47 (m, 2H), 0.43-0.37 (m, 2H).

### Step 2. Preparation of ethyl 1-(cyclopropylmethyl)-4,5,6,7-tetrahydro-1H-pyrazolo[4,3-c]pyridine-3-carboxylate (P31)

To a solution of C59 (0.200 g, 0.572 mmol) in DCM (0.5 mL), 2M HCl in dioxane (2.0 mL) was added. The reaction mixture was stirred at 35 °C for 3 h and then concentrated in vacuo to provide P31 (0.143 g, crude) as a grey solid. The solid was used directly in the next step without further purification.

### Preparation 32

### rac-Ethyl (R)-1-(1-methoxypropan-2-yl)-4,5,6,7-tetrahydro-1H-pyrazolo[4,3-c]pyridine-3-carboxylate hydrochloride (P32)

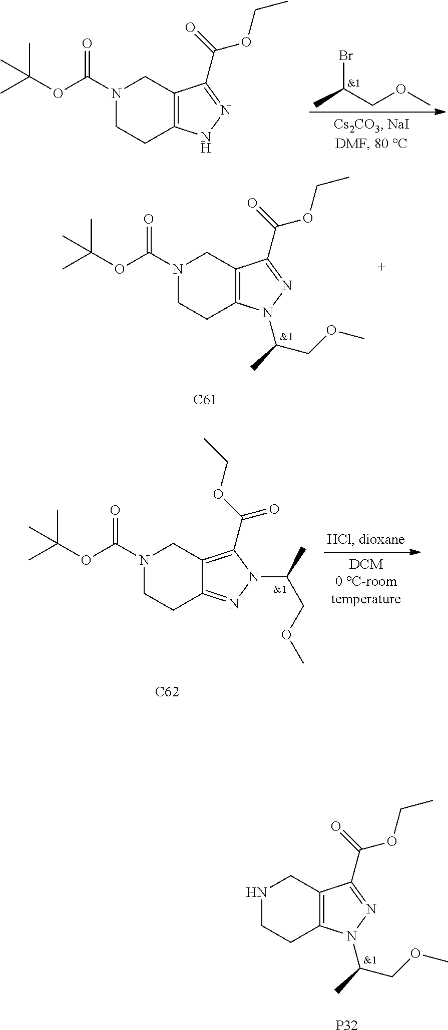

### Step 1. Preparation of rac-5-(tert-butyl) 3-ethyl (R)-1-(1-methoxypropan-2-yl)-1,4,6,7-tetrahydro-5H-pyrazolo[4,3-c]pyridine-3,5-dicarboxylate (C61) and rac-(R)-2-((3-(ethoxycarbonyl)-2-(1-methoxypropan-2-yl)-4,5,6,7-tetrahydro-2H-pyrazolo[4,3-c]pyridine-5-carbonyl)oxy)-2-methylpropan-1-ylium (C62)

The reaction was conducted in two batches then combined for purification. A solution of rac-(R)-2-bromo-1-methoxypropane (CAS: 22461-48-9; 0.829 g, 5.42 mmol), 5-(tert-butyl) 3-ethyl 1,4,6,7-tetrahydro-5H-pyrazolo[4,3-c]pyridine-3,5-dicarboxylate (CAS: 518990-23-3; 0.800 g, 2.71 mmol), Cs₂CO₃ (1.77 g, 5.42 mmol) and NaI (40.6 mg, 0.271 mmol) in DMF (13.5 mL) was stirred at 80 °C for 16 h to form the first batch.

The second batch of the same reaction was conducted with 5-(tert-butyl) 3-ethyl 1,4,6,7-tetrahydro-5H-pyrazolo[4,3-c]pyridine-3,5-dicarboxylate (CAS: 518990-23-3; 0.100 g, 0.339 mmol). The two batches were combined then diluted with H₂O (20 mL) and extracted with EtOAc (3×10 mL). The combined organic layer was dried Na₂SO₄, filtered, and concentrated in vacuo. The residue was purified by column chromatography (silica gel, 0-50% EtOAc: PE) to provide C62 (0.630 g, 56.2% yield) as an oil and C61 (0.430 g, 38.4% yield) as an oil.

C62: (LC/MS) m/z (M+H)⁺=368.2; ¹H NMR (400 MHz, CDCl₃) δ (ppm): 5.72-5.60 (m, 1H), 4.68-4.55 (m, 2H), 4.33 (q, 2H), 3.85 (t, 1H), 3.74-3.63 (m, 2H), 3.55 (dd, 1H), 3.31 (s, 3H), 2.79-2.71 (m, 2H), 1.48 (s, 9H), 1.45-1.36 (m, 6H).

C61: (LC/MS) m/z (M+H)⁺=368.2; ¹H NMR (400 MHz, CDCl₃) δ (ppm): 4.60 (s, 2H), 4.43-4.33 (m, 3H), 3.82-3.75 (m, 1H), 3.73-3.66 (m, 2H), 3.58 (dd, 1H), 3.24 (s, 3H), 2.83-2.62 (m, 2H), 1.52-1.46 (m, 12H), 1.38 (t, 3H).

### Step 2. Preparation of rac-ethyl (R)-1-(1-methoxypropan-2-yl)-4,5,6,7-tetrahydro-1H-pyrazolo[4,3-c]pyridine-3-carboxylate hydrochloride (P32)

At 0 °C, to a suspension of C61 (0.430 g, 1.17 mmol) in DCM (2 mL) was added HCl in dioxane (8 mL) in batches. The reaction mixture was stirred at room temperature for 2 h then concentrated in vacuo to provide P32 (0.356 g, crude) as a yellow solid. The solid was used directly in the next step without further purification. (LC/MS) m/z (M+H)⁺=268.1. ¹H NMR (400 MHz, (CD₃)₂SO) δ (ppm): 9.28-9.05 (m, 2H), 4.66-4.55 (m, 1H), 4.36-4.13 (m, 4H), 3.67-3.57 (m, 1H), 3.55-3.51 (m, 1H), 3.48-3.38 (m, 2H), 3.18 (s, 3H), 3.08-2.86 (m, 2H), 1.36 (d, 3H), 1.29 (t, 3H).

### Preparation 33

### Ethyl 1-(oxetan-3-yl)-4,5,6,7-tetrahydro-1H-pyrazolo[4,3-c]pyridine-3-carboxylate trifluoroacetate (P33)

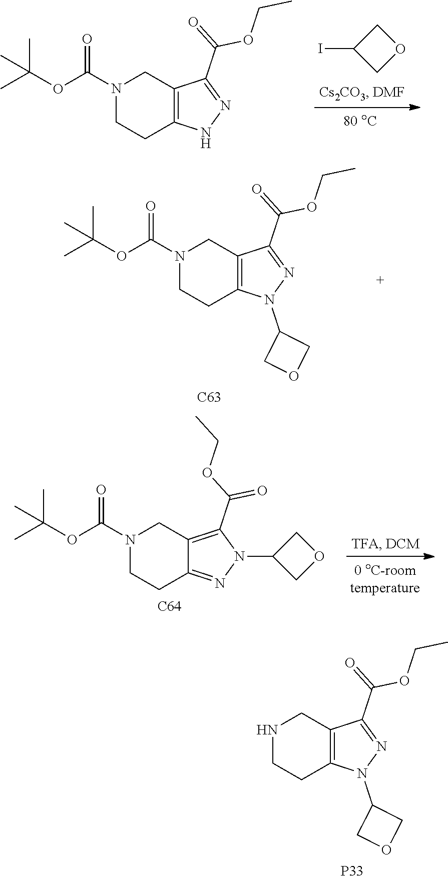

### Step 1. Preparation of 5-(tert-butyl) 3-ethyl 1-(oxetan-3-yl)-1,4,6,7-tetrahydro-5H-pyrazolo[4,3-c]pyridine-3,5-dicarboxylate (C63) and 2-((3-(ethoxycarbonyl)-2-(oxetan-3-yl)-4,5,6,7-tetrahydro-2H-pyrazolo[4,3-c]pyridine-5-carbonyl)oxy)-2-methylpropan-1-ylium (C64)

The reaction was conducted in two batches then combined for purification. To a solution of 5-(tert-butyl) 3-ethyl 1,4,6,7-tetrahydro-5H-pyrazolo[4,3-c]pyridine-3,5-dicarboxylate (CAS: 518990-23-3; 0.600 g, 2.03 mmol) in DMF (10 mL) was added 3-iodooxetane (CAS: 26272-85-5; 0.748 g, 4.06 mmol) and Cs₂CO₃ (1.32 g, 4.06 mmol) then stirred at 80 °C for 16 h to form the first batch.

The second batch of the same reaction conducted with 5-(tert-butyl) 3-ethyl 1,4,6,7-tetrahydro-5H-pyrazolo[4,3-c]pyridine-3,5-dicarboxylate (CAS: 518990-23-3; 0.100 g, 0.339 mmol) then the two batches were combined then diluted in ice H₂O (40 mL). The diluted reaction mixture was extracted with EtOAc (3×10 mL). The combined organic layer was washed with brine (2×10 mL) then concentrated in vacuo. The residue was purified by column chromatography (silica gel, 0-80% EtOAc: PE) to provide C63 (0.180 g, 25.2% yield) as an oil and C64 (0.288 g, 40.3% yield) as an oil.

C63 (Lot-001): (LC/MS) m/z (M+H)⁺=352.1; ¹H NMR (400 MHz, (CD₃)₂SO) δ (ppm): 5.61-5.51 (m, 1H), 4.89 (d, 4H), 4.49 (s, 2H), 4.33-4.24 (m, 2H), 3.58 (t, 2H), 2.68 (t, 2H), 1.41 (s, 9H), 1.31 (t, 3H).

C64: (LC/MS) m/z (M-tert-butyl group)+=296.1; ¹H NMR (400 MHz, (CD₃)₂SO) δ (ppm): 6.08-5.95 (m, 1H), 4.97-4.84 (m, 4H), 4.56-4.50 (m, 2H), 4.28 (q, 2H), 3.62 (t, 2H), 2.70 (t, 2H), 1.42 (s, 9H), 1.30 (t, 3H).

### Step 2. Preparation of ethyl 1-(oxetan-3-yl)-4,5,6,7-tetrahydro-1H-pyrazolo[4,3-c]pyridine-3-carboxylate trifluoroacetate (P33)

At 0 °C, to a suspension of C63 (0.180 g, 0.512 mmol) in DCM (3 mL) was added TFA (3 mL). The reaction mixture was stirred at room temperature for 3 h and then concentrated in vacuo to provide P33 (0.187 g, crude) as a gum. The gum was used directly in the next step without further purification. (LC/MS) m/z (M+H)⁺=252.3. ¹H NMR (400 MHz, (CD₃)₂SO) δ (ppm): 9.18-8.94 (m, 2H), 5.69-5.58 (m, 1H), 4.98-4.82 (m, 4H), 4.37-4.23 (m, 4H), 3.44-3.35 (m, 2H), 3.04-2.91 (m, 2H), 1.35-1.26 (m, 3H).

### Preparation 34

### Ethyl 1-(cyclobutylmethyl)-4,5,6,7-tetrahydro-1H-pyrazolo[4,3-c]pyridine-3-carboxylate hydrochloride

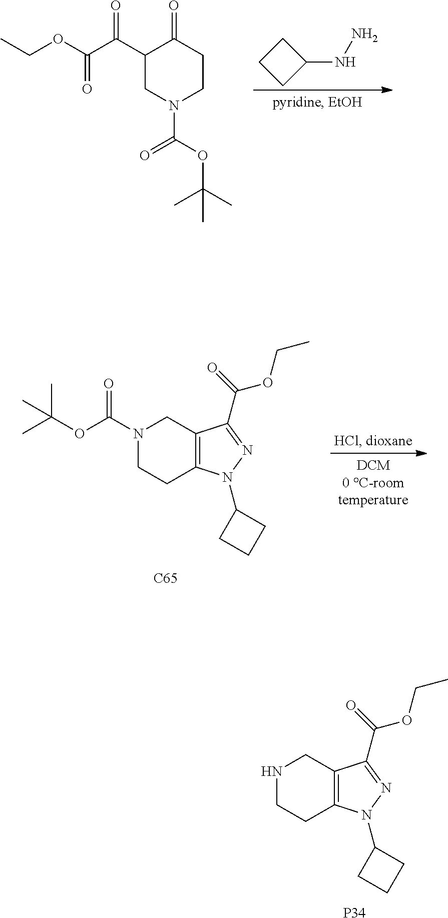

### Step 1. Preparation of 5-(tert-butyl) 3-ethyl 1-(cyclobutylmethyl)-1,4,6,7-tetrahydro-5H-pyrazolo[4,3-c]pyridine-3,5-dicarboxylate (C65)

The same procedure was followed from Preparation 2, step 1 with tert-butyl 3-(2-ethoxy-2-oxoacetyl)-4-oxopiperidine-1-carboxylate (CAS: 518990-24-4; 0.400 g, 1.34 mmol) and 1-cyclobutylhydrazine hydrochloride (CAS: 158001-21-9, 0.164 g, 1.34 mmol) to provide C65 (0.280 g, 15.0% yield) as a yellow gum. (LC/MS) m/z (M+H)⁺=350.2. ¹H NMR (400 MHz, (CD₃)₂SO) δ (ppm): 4.90-4.75 (m, 1H), 4.48 (s, 2H), 4.28 (q, 2H), 3.60 (t, 2H), 2.73-2.66 (m, 2H), 2.40-2.30 (m, 4H), 1.87-1.74 (m, 2H), 1.46-1.35 (m, 9H), 1.30 (t, 3H).

### Step 2. Preparation of ethyl 1-(cyclobutylmethyl)-4,5,6,7-tetrahydro-1H-pyrazolo[4,3-c]pyridine-3-carboxylate hydrochloride (P34)

At 0 °C, HCl was added to a suspension of C65 (0.280 g, 0.801 mmol) in DCM (5 mL) in dioxane (10 mL). The reaction mixture was stirred at room temperature for 3 h, concentrated in vacuo, and then lyophilized to provide P34 (0.150 g, crude) as a brown solid. The solid was used directly in the next step without further purification. (LC/MS) m/z (M+H)⁺=250.1. ¹H NMR (400 MHz, (CD₃)₂SO) δ (ppm): 9.37 (s, 2H), 4.94-4.80 (m, 1H), 4.29 (q, 2H), 4.21 (s, 2H), 3.42-3.36 (m, 2H), 2.95 (t, 2H), 2.48-2.44 (m, 1H), 2.41-2.31 (m, 2H), 1.87-1.75 (m, 2H), 1.30 (t, 3H).

### Preparation 35

### rac-Ethyl (R)-1-(1,1,1-trifluoropropan-2-yl)-4,5,6,7-tetrahydro-1H-pyrazolo[4,3-c]pyridine-3-carboxylate (P35)

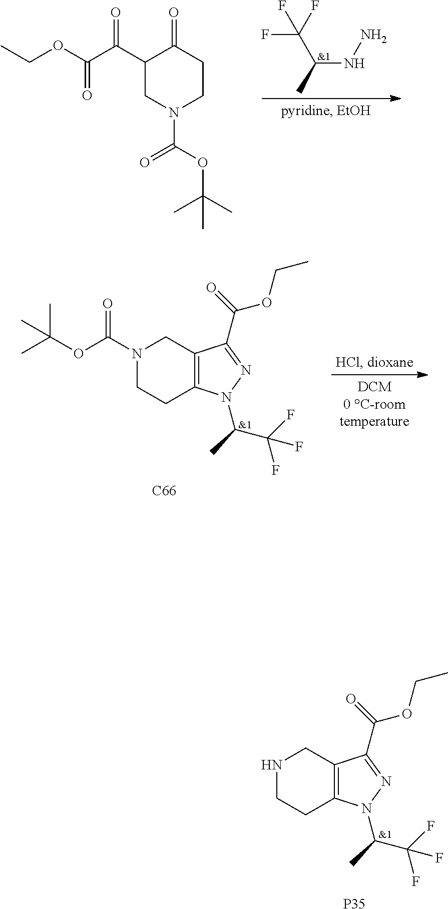

### Step 1. Preparation of rac-5-(tert-butyl) 3-ethyl (R)-1-(1,1,1-trifluoropropan-2-yl)-1,4,6,7-tetrahydro-5H-pyrazolo[4,3-c]pyridine-3,5-dicarboxylate (C66)

To a solution of tert-butyl 3-(2-ethoxy-2-oxoacetyl)-4-oxopiperidine-1-carboxylate (CAS: 518990-24-4; 0.400 g, 1.34 mmol) and (1,1,1-trifluoropropan-2-yl)hydrazine hydrochloride (CAS: 1453472-98-4; 0.269 g, 1.34 mmol) in EtOH (7 mL) was added pyridine (0.465 g, 5.88 mmol). The reaction mixture was stirred at room temperature for 5 h and then concentrated in vacuo. The residue was purified by column chromatography (silica gel, 0-25% EtOAc: PE) to provide C66 (0.415 g, 79.3% yield) as a yellow gum. (LC/MS) m/z (M+H)⁺=392.2. ¹H NMR (400 MHz, (CD₃)₂SO) δ (ppm): 5.47-5.35 (m, 1H), 4.60-4.38 (m, 2H), 4.29 (q, 2H), 3.78-3.67 (m, 1H), 3.55-3.46 (m, 1H), 2.92-2.81 (m, 1H), 2.69-2.56 (m, 1H), 1.67 (d, 3H), 1.41 (s, 9H), 1.30 (t, 3H).

### Step 2. Preparation of rac-ethyl (R)-1-(1,1,1-trifluoropropan-2-yl)-4,5,6,7-tetrahydro-1H-pyrazolo[4,3-c]pyridine-3-carboxylate (P35)

At 0 °C, to a suspension of C66 (0.415 g, 1.06 mmol) in DCM (4 mL) was added HCl in dioxane (8 mL) in batches. The reaction mixture was stirred at room temperature for 16 h then concentrated in vacuo to provide P35 (0.306 g, crude) as a yellow solid. The solid was used directly in the next step without further purification.

### Preparation 36

### Ethyl 1,7,7-trimethyl-4,5,6,7-tetrahydro-1H-pyrazolo[4,3-c]pyridine-3-carboxylate hydrochloride (P36)

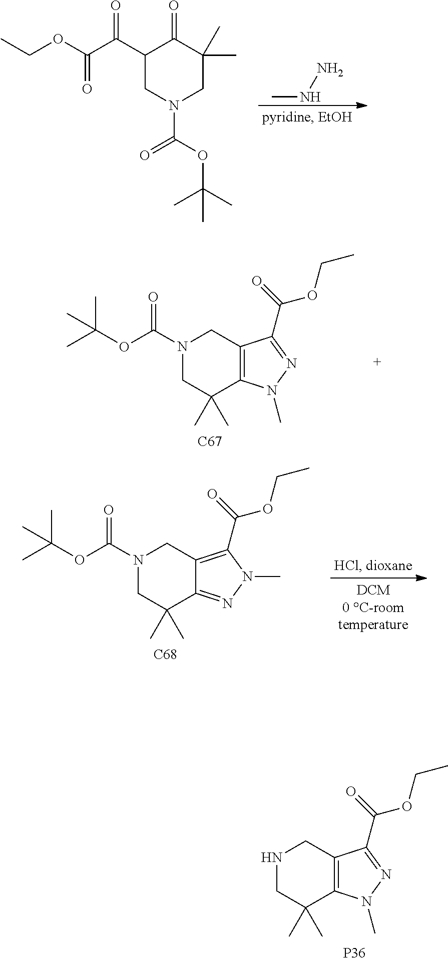

### Step 1. Preparation of 5-(tert-butyl) 3-ethyl 1,7,7-trimethyl-1,4,6,7-tetrahydro-5H-pyrazolo[4,3-c]pyridine-3,5-dicarboxylate (C67) and 2-((3-(ethoxycarbonyl)-2,7,7-trimethyl-4,5,6,7-tetrahydro-2H-pyrazolo[4,3-c]pyridine-5-carbonyl)oxy)-2-methylpropan-1-ylium (C68)

The same procedure was followed from Preparation 2, step 1 with tert-butyl 5-(2-ethoxy-2-oxoacetyl)-3,3-dimethyl-4-oxopiperidine-1-carboxylate (CAS: 2171295-98-8, 1.28 g, 3.91 mmol) and methylhydrazine (CAS: 60-34-4, 1.08 g, 9.38 mmol) to provide C68 (0.563 g, 42.7% yield) as a light-yellow gum and C67 (0.160 g, 12.1% yield) as a light-yellow gum.

C67: (LC/MS) m/z (M+H)⁺=338.2; ¹H NMR (400 MHz, CDCl₃) δ (ppm): 4.66-4.54 (m, 2H), 4.44-4.30 (m, 2H), 4.00-3.92 (m, 3H), 3.45-3.34 (m, 2H), 1.51-1.45 (m, 9H), 1.41-1.35 (m, 3H), 1.34-1.29 (m, 6H).

C68: (LC/MS) m/z (M+H)⁺=338.1; ¹H NMR (400 MHz, CDCl₃) δ (ppm): 4.66-4.56 (m, 2H), 4.38-4.26 (m, 2H), 4.16-4.05 (m, 3H), 3.45-3.32 (m, 2H), 1.49-1.46 (m, 9H), 1.37 (t, 3H), 1.28-1.24 (m, 6H).

### Step 2. Preparation of ethyl 1,7,7-trimethyl-4,5,6,7-tetrahydro-1H-pyrazolo[4,3-c]pyridine-3-carboxylate hydrochloride (P36)

At 0 °C, to a suspension of C67 (0.130 g, 0.385 mmol) in DCM (3 mL) was added HCl in dioxane (3 mL). The reaction mixture was stirred at room temperature for 2 h and then concentrated in vacuo to provide P36 (0.110 g, crude) as a yellow solid. The solid was used directly in the next step without further purification. (LC/MS) m/z (M+H)⁺=238.1. ¹H NMR (400 MHz, (CD₃)₂SO) δ (ppm): 9.58 (s, 2H), 4.27 (q, 2H), 4.18 (s, 2H), 3.98 (s, 3H), 3.21-3.16 (m, 2H), 1.42 (s, 6H), 1.31-1.22 (m, 3H).

### Preparation 37

### Ethyl 1′-methyl-1′,4′,5′,6′-tetrahydrospiro[cyclopropane-1,7′-pyrazolo[4,3-c]pyridine]-3′-carboxylate hydrochloride (P37)

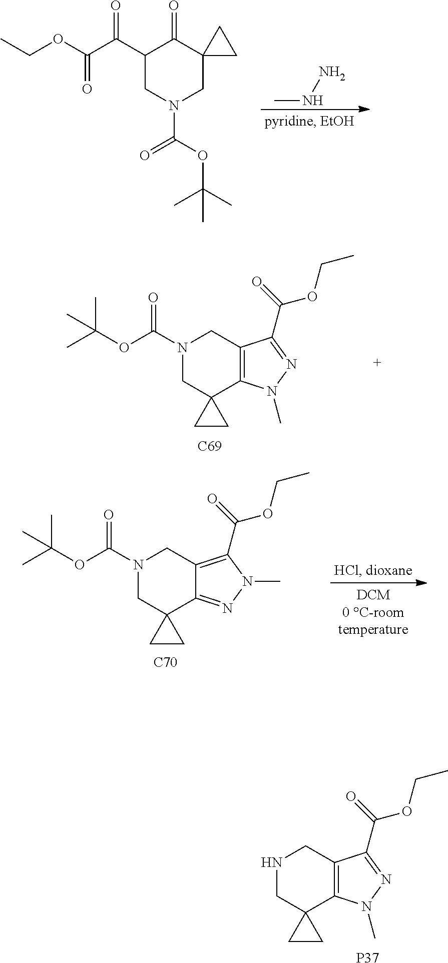

### Step 1. Preparation of 5′-(tert-butyl) 3′-ethyl 1′-methyl-1′,4′-dihydrospiro[cyclopropane-1,7′-pyrazolo[4,3-c]pyridine]-3′,5′(6′H)-dicarboxylate (C69) and 2-((3′-(ethoxycarbonyl)-2′-methyl-2′,4′,5′,6′-tetrahydrospiro[cyclopropane-1,7′-pyrazolo[4,3-c]pyridine]-5′-carbonyl)oxy)-2-methylpropan-1-ylium (C70)

The same procedure was followed from Preparation 2, step 1 with tert-butyl 7-(2-ethoxy-2-oxoacetyl)-8-oxo-5-azaspiro[2.5]octane-5-carboxylate (CAS: 2494304-89-9; 0.620 g, 1.91 mmol) and methylhydrazine sulfate (CAS: 302-15-8, 0.302 g, 2.10 mmol). The purification was altered. The residue was purified by column chromatography (silica gel, 0-30% THF: PE) to provide C70 (0.327 g, 51.2% yield) as a white solid and C69 (0.290 g, 45.4% yield) as a white gum.

C69: (LC/MS) m/z (M+H)⁺=336.3; ¹H NMR (400 MHz, CDCl₃) δ (ppm): 4.70 (s, 2H), 4.39 (q, 2H), 3.74 (s, 3H), 3.44 (s, 2H), 1.48 (s, 9H), 1.39 (t, 3H), 1.31 (t, 2H), 1.08-0.94 (m, 2H).

C70: (LC/MS) m/z (M+H)⁺=336.3; ¹H NMR (400 MHz, CDCl₃) δ (ppm): 4.75-4.65 (m, 2H), 4.34 (q, 2H), 4.08 (s, 3H), 3.53-3.47 (m, 2H), 1.48 (s, 9H), 1.39 (t, 3H), 1.12 (q, 2H), 0.97-0.86 (m, 2H).

### Step 2. Preparation of ethyl 1′-methyl-1′,4′,5′,6′-tetrahydrospiro[cyclopropane-1,7′-pyrazolo[4,3-c]pyridine]-3′-carboxylate hydrochloride (P37)

At 0 °C, to a suspension of C69 (0.272 g, 0.811 mmol) in DCM (6 mL) was added HCl in dioxane (0.591 g, 16.2 mmol). The reaction mixture was stirred at room temperature for 3 h then concentrated in vacuo to provide P37 (0.219 g, crude) as a white solid. The solid was used directly in the next step without further purification.

### Preparation 38

### rac-Ethyl (R)-1-isopropyl-7-methyl-4,5,6,7-tetrahydro-1H-pyrazolo[4,3-c]pyridine-3-carboxylate

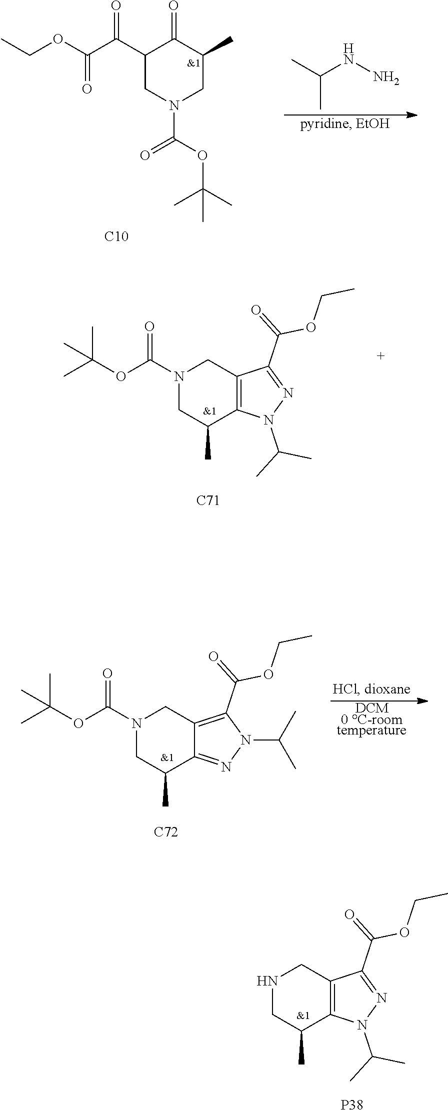

### Step 1. Preparation of rac-5-(tert-butyl) 3-ethyl (R)-1-isopropyl-7-methyl-1,4,6,7-tetrahydro-5H-pyrazolo[4,3-c]pyridine-3,5-dicarboxylate (C71) and rac-5-(tert-butyl) 3-ethyl (R)-2-isopropyl-7-methyl-2,4,6,7-tetrahydro-5H-pyrazolo[4,3-c]pyridine-3,5-dicarboxylate (C72)

The same procedure was followed from Preparation 2, step 1 with C10 (3.00 g, 9.57 mmol) and isopropylhydrazine hydrochloride (CAS: 16726-41-3, 1.06 g, 9.57 mmol). The purification was altered. The residue was purified by reverse phase HPLC (Sunfire C₁₈ 150 mm×40 mm×5 μm column, Mobile Phase A: H₂O (0.05% NH₄OH—NH₄HCO₃)/Mobile Phase B: ACN, Mobile Phase B: 42 to 82% over 9.0 min, 100% of Mobile Phase B hold 2.0 min, flow rate=30 mL/min) to provide C71 (1.80 g, 53.5% yield) as a pale yellow solid and C72 (33.9 mg, 1.01% yield) as a pale yellow solid.

C71: (LC/MS) m/z (M+H)⁺=352.4. ¹H NMR (400 MHz, CDCl₃) δ (ppm): 5.05 (dd, 1H), 4.46-4.31 (m, 3H), 4.13 (dd, 2H), 3.20-3.04 (m, 1H), 2.99-2.89 (m, 1H), 1.54 (d, 3H), 1.51-1.45 (m, 12H), 1.36 (t, 3H), 1.23 (d, 3H).

C72: (LC/MS) m/z (M+H)⁺=352.4. ¹H NMR (400 MHz, CDCl₃) δ (ppm): 5.56-5.44 (m, 1H), 4.69-4.45 (m, 2H), 4.32 (q, 2H), 3.90-3.61 (m, 1H), 3.41-3.13 (m, 1H), 3.02-2.92 (m, 1H), 1.51-1.44 (m, 15H), 1.38 (t, 3H), 1.26 (d, 3H).

### Step 2. Preparation of rac-ethyl (R)-1-isopropyl-7-methyl-4,5,6,7-tetrahydro-1H-pyrazolo[4,3-c]pyridine-3-carboxylate hydrochloride (P38)

At 0 °C, to a suspension of C71 (0.200 g, 0.569 mmol) in DCM (1 mL) was added HCl in dioxane (4 mL). The reaction mixture was stirred at room temperature for 2 h then concentrated in vacuo to provide P38 (0.180 g, crude) as a yellow solid. The solid was used directly in the next step without further purification. (LC/MS) m/z (M+H)⁺=252.1. ¹H NMR (400 MHz, (CD₃)₂SO) δ (ppm): 9.98-9.83 (m, 1H), 9.34-9.25 (m, 1H), 4.67-4.54 (m, 1H), 4.33-4.05 (m, 4H), 3.43-3.26 (m, 3H), 1.45-1.40 (m, 3H), 1.39-1.33 (m, 6H), 1.32-1.26 (m, 3H).

### Preparation 39

### 2-Fluoro-5-(1-methyl-1H-1,2,4-triazol-5-yl)pyridine (P39)

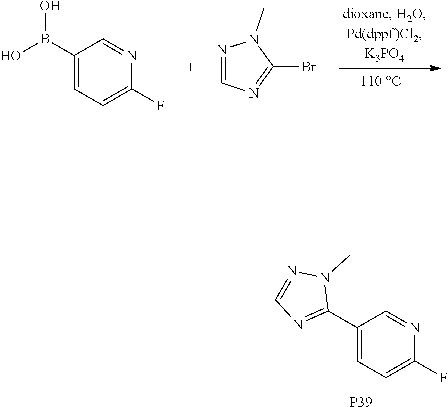

### Step 1. Preparation of 2-fluoro-5-(1-methyl-1H-1,2,4-triazol-5-yl)pyridine (P39)

To a suspension of (6-fluoropyridin-3-yl)boronic acid (CAS: 351019-18-6; 1.10 g, 7.81 mmol) and 5-bromo-1-methyl-1H-1,2,4-triazole (CAS: 16681-72-4; 1.52 g, 9.37 mmol) in dioxane (10 mL) and H₂O (2 mL) was added Pd(dppf)Cl₂ (0.457 g, 0.625 mmol) and K₃PO₄ (4.97 g, 23.4 mmol). The reaction mixture was degassed with nitrogen gas, then stirred at 110 °C for 4 h and purified by column chromatography (silica gel, 0-40% EtOAc: PE) to provide P39 (1.20 g, 86.3% yield) as a white solid. (LC/MS) m/z (M+H)⁺=179.1. ¹H NMR (400 MHz, (CD₃)₂SO) δ (ppm): 8.67-8.62 (m, 1H), 8.42-8.34 (m, 1H), 8.05 (s, 1H), 7.40 (dd, 1H), 3.97 (s, 3H).

### Preparation 40

### 2-Fluoro-5-(1-methyl-1H-imidazol-2-yl)pyridine (P40)

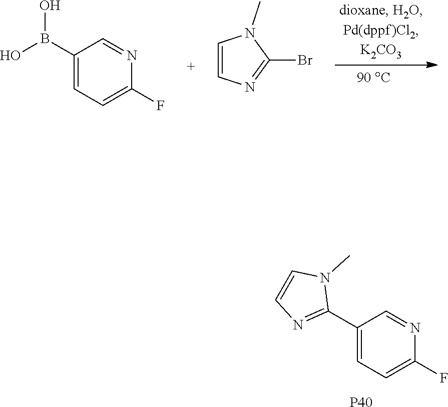

### Step 1. Preparation of 2-fluoro-5-(1-methyl-1H-imidazol-2-yl)pyridine (P40)

Under nitrogen gas, the reaction mixture of 2-bromo-1-methyl-1H-imidazole (0.700 g, 4.35 mmol), (6-fluoropyridin-3-yl)boronic acid (1.20 g, 8.70 mmol) and Pd(dppf)Cl₂ (0.178 g, 0.217 mmol) suspended in dioxane (10 mL) and H₂O (2 mL) was stirred at 90 °C for 1 h. The reaction mixture 25 was extracted with EtOAc, and then the organic layer was concentrated in vacuo. The residue was dissolved in DCM and then purified by column chromatography (silica gel, 0-10% MeOH: DCM) to provide P40 (0.688 g, 89.3% yield) as an orange oil. (LC/MS) m/z (M+H)⁺=178.1.

### Preparation 41

### 2-(4-(Aminomethyl)phenyl)pyrimidin-4(3H)-one hydrochloride (P41)

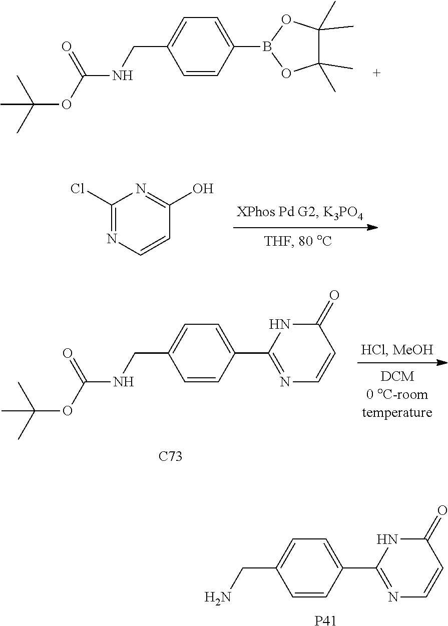

Step 1. Preparation of tert-butyl (4-(6-oxo-1,6-dihydropyrimidin-2-yl)benzyl)carbamate (C73) To a solution of 2-chloropyrimidin-4-ol (CAS: 55873-09-1; 0.100 g, 0.766 mmol) and tert-butyl (4-(4,4,5,5-tetramethyl-1,3,2-dioxaborolan-2-yl)benzyl)carbamate (CAS: 330794-35-9; 0.255 g, 0.766 mmol) in THF (7.7 mL) was added 2M K₃PO₄ (0.325 g, 1.53 mmol). The reaction mixture was degassed with nitrogen gas for 3 min, and then XPhos Pd G2 (60.3 mg, 0.0766 mmol) was added. The suspension was degassed with nitrogen gas for 3 min and then stirred at 80 °C for 4 h. The reaction solution was diluted with H₂O (10 mL) and then extracted with MeOH:DCM (1:10, 3×5 mL). The combined organic layer was dried with Na₂SO₄, filtered, and the filtrate was concentrated in vacuo. The residue was purified by column chromatography (silica gel, 0-100% EtOAc: PE) to provide C73 (0.115 g, 49.8% yield) as a white solid. (LC/MS) m/z (M+H)⁺=302.1. ¹H NMR (400 MHz, (CD₃)₂SO) δ (ppm): 12.75 (br s, 1H), 8.16-7.92 (m, 3H), 7.51-7.44 (m, 1H), 7.36 (d, 2H), 6.36-6.24 (m, 1H), 4.18 (d, 2H), 1.39 (s, 9H).

### Step 2. Preparation of 2-(4-(aminomethyl)phenyl)pyrimidin-4(3H)-one hydrochloride (P41)

At 0 °C, to a stirred solution of C73 (0.115 g, 0.382 mmol) in DCM (1 mL) was added HCl in MeOH (3 mL). The reaction mixture was stirred at room temperature for 2 h and then concentrated in vacuo to provide P41 (91.0 mg, crude) as a white solid. The solid was used directly in the next step without further purification. ¹H NMR (400 MHz, (CD₃)₂SO) δ (ppm): 11.05-10.97 (m, 1H), 10.88-10.81 (m, 1H), 8.60-8.47 (m, 2H), 8.17-8.08 (m, 2H), 7.66-7.61 (m, 2H), 7.39 (dd, 1H), 6.40-6.36 (m, 1H), 5.46-5.42 (m, 1H), 4.10 (q, 2H).

### Preparation 42

### 6-(4-(Aminomethyl)phenyl)pyridin-2(1H)-one hydrochloride (P42)

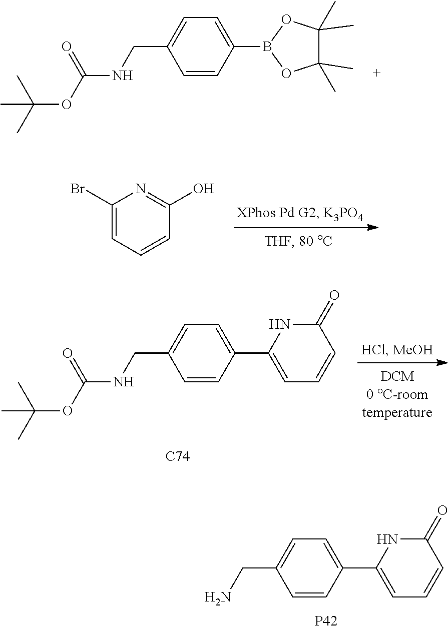

### Step 1. Preparation of tert-butyl (4-(6-oxo-1,6-dihydropyridin-2-yl)benzyl)carbamate (C74)

To a solution of 6-bromopyridin-2-ol (CAS: 27992-32-1; 0.100 g, 0.575 mmol) and tert-butyl (4-(4,4,5,5-tetramethyl-1,3,2-dioxaborolan-2-yl)benzyl)carbamate (CAS: 330794-35-9; 0.192 g, 0.575 mmol) in THF (5.7 mL) was added 2M K₃PO₄ (0.244 g, 1.15 mmol). The reaction mixture was degassed with nitrogen gas for 3 min, then XPhos Pd G2 (45.2 mg, 0.0575 mmol) was added. The suspension was degassed with nitrogen gas for 3 min and then stirred at 80 °C for 4 h. The reaction solution was diluted with H₂O (10 mL) and then extracted with EtOAc (3×5 mL). The combined organic layer was dried with Na₂SO₄, filtered, and the filtrate was concentrated in vacuo. The residue was purified by column chromatography (silica gel, 0-100% EtOAc: PE) to provide C74 (0.110 g, 63.7% yield) as a white solid. (LC/MS) m/z (M+H)⁺=301.1. ¹H NMR (400 MHz, (CD₃)₂SO) δ (ppm): 11.71 (br s, 1H), 7.73 (d, 2H), 7.58-7.51 (m, 1H), 7.49-7.44 (m, 1H), 7.33 (d, 2H), 6.71-6.57 (m, 1H), 6.37 (d, 1H), 4.18 (d, 2H), 1.41 (s, 9H).

### Step 2. Preparation of 6-(4-(aminomethyl)phenyl)pyridin-2(1H)-one hydrochloride (P42)

At 0 °C, to a stirred solution of C74 (0.110 g, 0.366 mmol) in DCM (1 mL) was added HCl in MeOH (3 mL). The reaction mixture was stirred at room temperature for 2 h and then concentrated in vacuo to provide P42 (87.0 mg, crude) as a white solid. The solid was used directly in the next step without further purification. ¹H NMR (400 MHz, (CD₃)₂SO) δ (ppm): 8.46-8.36 (m, 3H), 7.84 (d, 2H), 7.61-7.54 (m, 3H), 6.80-6.70 (m, 1H), 6.43 (d, 1H), 4.07 (q, 2H).

### Preparation 43

### 3-(4-(Aminomethyl)phenyl-2,3,5,6-d₄)-1,1-dimethylurea hydrochloride (P43)

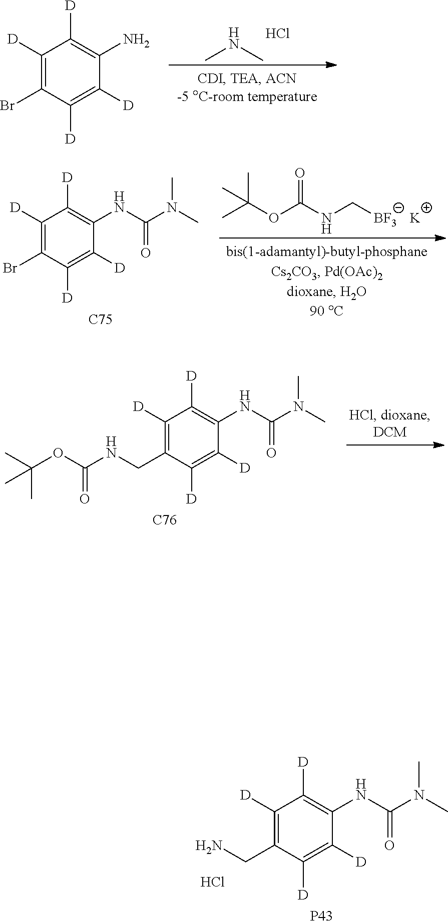

### Step 1. Preparation of 3-(4-bromophenyl-2,3,5,6-d₄)-1,1-dimethylurea (C75)

To a solution of CDI (6.59 g, 40.6 mmol) in ACN (50 mL) was added 4-bromobenzen-2,3,5,6-d₄-amine (CAS: 61357-76-4; 5.50 g, 31.2 mmol) at (−5 to 5) °C. The reaction mixture was stirred from (0 to 5) °C for 3 h before TEA (6.32 g, 62.5 mmol) and dimethylamine hydrochloride (CAS: 506-59-2; 5.10 g, 62.5 mmol) were added slowly at (0 to 5) °C. The reaction mixture was warmed to room temperature and stirred for 16 h. The suspension was poured into H₂O (100 mL) and extracted with EtOAc (3×100 mL). The combined organic layer was washed with brine (200 mL), dried over MgSO₄, and concentrated in vacuo. The residue was slurried with MTBE: DCM (10:1, 100 mL) at room temperature for 3 h and then filtered. The filter cake was dried in vacuo and then collected to provide C75 (6.10 g, 76.2% yield) as a white solid. ¹H NMR (400 MHz, (CD₃)₂SO) δ (ppm): 8.40 (s, 1H), 2.91 (s, 6H).

### Step 2. Preparation of tert-butyl (4-(3,3-dimethylureido)2,3,5,6-d₄-benzyl)carbamate (C76)

A reaction mixture of C75 (3.0 g, 12 mmol), potassium [[(tert-butoxycarbonyl)amino]methyl]trifluoroborate (4.3 g, 18 mmol) and Cs₂CO₃ (7.9 g, 24 mmol) in H₂O (6 mL) and dioxane (40 mL) was degassed and purged with nitrogen gas 3 times. To the reaction mixture were added bis(1-adamantyl)-butyl-phosphane (0.87 g, 2.4 mmol) and Pd(OAc)₂ (0.27 g, 1.2 mmol). The suspension was degassed and purged with nitrogen gas 3 times again, then stirred at 90 °C for 16 h under nitrogen gas. The reaction mixture was cooled to room temperature and then poured into H₂O (50 mL) and filtered. The filtrate was extracted with EtOAc (3×50 mL). The combined organic layer was washed with brine (100 mL), dried over MgSO₄, and concentrated in vacuo. The residue was slurried with DCM (100 mL) at room temperature for 4 h and then filtered. The filter cake was dried in vacuo and then collected to provide C76 (2.0 g, 30.% yield) as a light-yellow solid.

### Step 3. Preparation of 3-(4-(aminomethyl)phenyl-2,3,5,6-d₄)-1,1-dimethylurea hydrochloride (P43)

To a solution of C76 (2.0 g, 6.7 mmol) in DCM (30 mL) was added 2M HCl in dioxane (25 mL). The reaction mixture was stirred at room temperature for 16 h and then concentrated in vacuo. The residue was purified by reverse phase HPLC (Welch Xtimate C₁₈ 250 mm×50 mm×10 μm column, Mobile Phase A: H₂O with HCl/Mobile Phase B: ACN, 0 to 20% of Mobile Phase B over 20 min) then lyophilized to provide P43 (1.1 g, 67% yield) as a white solid. ¹H NMR (400 MHz, (CD₃)₂SO) δ (ppm): 8.43 (s, 1H), 8.37 (br s, 2H), 3.90 (q, 2H), 2.92 (s, 6H).

### Preparation 44

### rac-Ethyl (R)-1-isopropyl-7-(trifluoromethyl)-4,5,6,7-tetrahydro-1H-pyrazolo[4,3-c]pyridine-3-carboxylate hydrochloride (P44)

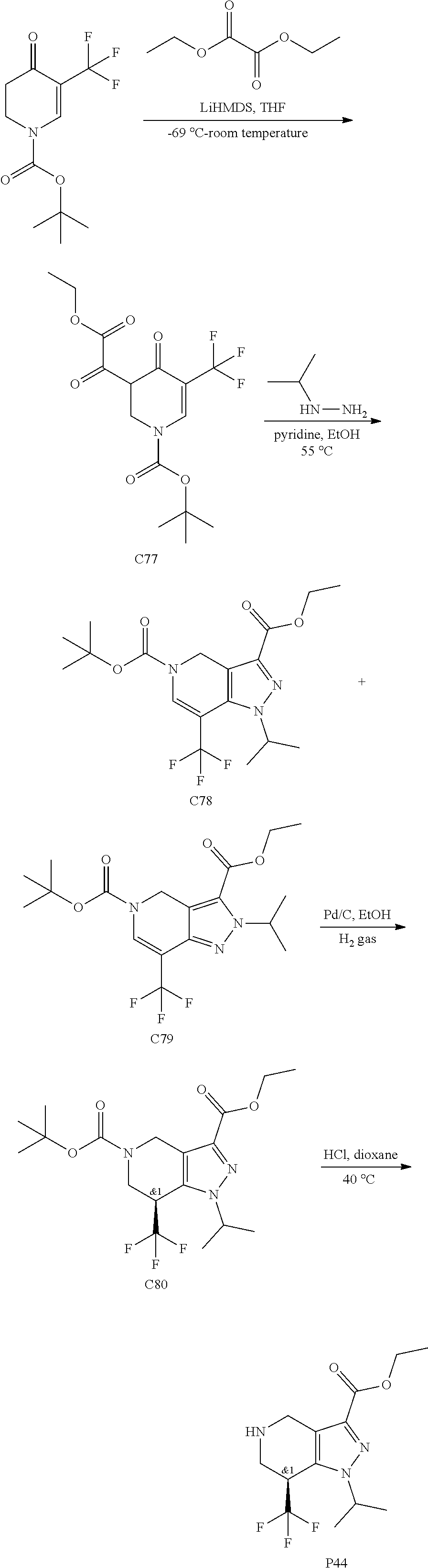

### Step 1. Preparation of tert-butyl 3-(2-ethoxy-2-oxoacetyl)-4-oxo-5-(trifluoromethyl)-3,4-dihydropyridine-1(2H)-carboxylate (C77)

A yellow solution of 1M LiHMDS in THF (18.4 mL) was diluted with THF (10 mL) then cooled to −69 °C. with a dry ice/acetone bath. To the cooled solution was added tert-butyl 4-oxo-5-(trifluoromethyl)-3,4-dihydropyridine-1(2H)-carboxylate (CAS: 1667744-92-4; 4.07 g, 15.4 mmol) in THF (20 mL) dropwise over 15 min. The substrate flask was rinsed with THF (2×1 mL) and added to the reaction mixture. During the addition, the solution became a brownish-orange color. The reaction was stirred at −69 °C for 45 min after the addition. A solution of diethyl oxalate (2.52 mL) in THF (5 mL) was added dropwise to the reaction mixture over 8 min. The source vial was rinsed with THF (1.5 mL), which was then added to the reaction mixture, which changed the solution to a brownish-orange color. The reaction mixture was stirred in the dry ice/acetone bath for another 7 min, then removed and replaced with an ice water bath. The reaction mixture was quenched with aqueous 2N HCl (21 mL), which changed the color of the suspension to an orange color. The suspension was diluted with H₂O and extracted with EtOAc (2×15 mL). The combined organic layers were washed with saturated NaHCO₃ solution (1×20 mL, 2×10 mL). The organic layers were set aside. The aqueous layers were combined and washed with MTBE (3×20 mL). The combined organic layers were set aside. The aqueous layer was acidified with 6N HCl (-6.5 mL) to pH=1. The acidic aqueous layer was extracted with MTBE (2×40 mL). The combined organic layer from the acidic aqueous layer extraction was dried with Na₂SO₄, filtered, and concentrated in vacuo, then dried further by high vacuum to provide C77 (1.52 g) as an orange-yellow residue.

The organic layers that were set aside were combined. The combined organic layer was dried with Na₂SO₄, filtered, and concentrated in vacuo to give a deep orange oil. The oil was dissolved in MTBE (40 mL) and then washed with saturated NaHCO₃ solution (1×32 mL, 1×10 mL). The aqueous layers were combined and acidified with aqueous 6N HCl. The acidic aqueous layer was extracted with MTBE (30 mL) and then MTBE (20 mL). The organic layers were combined and then dried with Na₂SO₄, filtered, concentrated in vacuo, and dried further by high vacuum to provide C77 (1.92 g) as an orange residue.

The two batches of products were combined to form C77 (3.44 g) and then purified by column chromatography (silica gel, 0-85% EtOAc: heptane) to provide C77 (2.32 g, 41.3% yield) as a yellow oil. (LC/MS) m/z (M+H)⁺=366.5. ¹H NMR (400 MHz, CDCl₃) δ (ppm): 15.18 (s, 1H), 8.28 (s, 1H), 5.03 (s, 2H), 4.38 (q, 2H), 1.58 (s, 9H), 1.40 (t, 3H).

### Step 2. Preparation of 5-(tert-butyl) 3-ethyl 1-isopropyl-7-(trifluoromethyl)-1,4,6,7-tetrahydro-5H-pyrazolo[4,3-c]pyridine-3,5-dicarboxylate (C78) and 2-((3-(ethoxycarbonyl)-2-isopropyl-7-(trifluoromethyl)-4,5,6,7-tetrahydro-2H-pyrazolo[4,3-c]pyridine-5-carbonyl)oxy)-2-methylpropan-1-ylium (C79)

To a solution of C77 (408 mg, 1.12 mmol) in EtOH (4.5 mL) was added isopropylhydrazine hydrochloride (CAS: 16726-41-3; 148 mg, 1.34 mmol) and then pyridine (132 mg, 1.67 mmol). The reaction mixture was stirred at 55 °C for 20 h and then concentrated in vacuo to give a yellow-orange residue. The residue was diluted with EtOAc (20 mL) and washed with NaHCO₃ (20 mL). The aqueous layer was back-extracted with EtOAc (2×10 mL). The combined organic layers were washed with brine, dried with Na₂SO₄, filtered, and concentrated in vacuo to give an orange oil. The oil was dissolved in DCM then preabsorbed on (1:1) silica gel: Celite and purified by column chromatography (silica gel, 0-100% EtOAc: heptane) to provide C78 (0.315 g, 69.9% yield) as a gum and C79 (7.70 mg, 1.71% yield) as a light-yellow solid.

C78: (LC/MS) m/z (M+H)⁺=404.6; ¹H NMR (400 MHz, CDCl₃) δ (ppm): 7.67-7.46 (m, 1H), 5.02 (s, 2H), 4.73-4.63 (m, 1H), 4.39 (q, 2H), 1.55 (s, 9H), 1.50 (d, 6H), 1.39 (t, 3H).

C79: (LC/MS) m/z (M+H)⁺=404.5; ¹H NMR (500 MHz, CDCl₃) δ (ppm): 7.63-7.33 (m, 1H), 5.51-5.49 (m, 1H), 4.97 (s, 2H), 4.35 (q, 2H), 1.55 (s, 9H), 1.48 (d, 6H), 1.39 (t, 3H).

### Step 3. Preparation of rac-5-(tert-butyl) 3-ethyl (R)-1-isopropyl-7-(trifluoromethyl)-1,4,6,7-tetrahydro-5H-pyrazolo[4,3-c]pyridine-3,5-dicarboxylate (C80)

To a suspension of Pd/C (CAS: 7440-05-3; 83 mg, 5% wt, 0.039 mmol) in EtOH (0.5 mL) was added a light-yellow solution of C78 (0.30 g, 0.75 mmol) in EtOH (1.5 mL). The source vial was rinsed with EtOH (0.5 mL×2), and then the rinses were added to the reaction mixture for a total volume of EtOH (3 mL). The reaction mixture was stirred at room temperature overnight in a Hastelloy reactor at H₂ gas (50 psi). The suspension was filtered through Celite (0.5 g) with a prepacked plastic filter funnel. The filter cake was eluted with EtOH. The grey filtrate was concentrated in vacuo to give a grey residue.

The same reaction was set up again. To a reaction mixture of Pd/C (98 mg, 5 wt %, 0.046 mmol) in EtOH (0.5 mL) was added the solution of the crude residue dissolved in EtOH (1.5 mL). The reaction mixture was stirred at room temperature overnight in a Hastelloy reactor at H₂ gas (50 psi). The suspension was filtered through Celite (0.5 g) with a prepacked plastic filter funnel. The filter cake was eluted with EtOH. The grey filtrate was concentrated in vacuo to provide a grey solid. The solid was purified by column chromatography (silica gel, 0-50% EtOAc: heptane) to provide C80 (0.24 g, 78% yield) as a white solid. (LC/MS) m/z (M+H)⁺=406.6; ¹H NMR (400 MHz, CDCl₃) δ (ppm): 5.35-5.23 (m, 1H), 5.13-4.76 (m, 2H), 4.59-4.47 (m, 1H), 4.44-4.34 (m, 3H), 4.29-4.08 (m, 2H), 3.60-3.44 (m, 1H), 3.08 (dd, 1H), 1.59-1.55 (m, 9H), 1.43-1.35 (m, 6H).

### Step 4. Preparation of rac-ethyl (R)-1-isopropyl-7-(trifluoromethyl)-4,5,6,7-tetrahydro-1H-pyrazolo[4,3-c]pyridine-3-carboxylate hydrochloride (P44)

To C80 (0.234 g, 0.577 mmol) was added 4N HCl in dioxane (1.44 mL) dropwise. The reaction mixture was stirred at 40 °C for 40 min then concentrated under nitrogen gas then dried further under high vacuum to provide P44 (0.210 g, crude) as a white solid. (LC/MS) m/z (M+H)⁺=306.5; ¹H NMR (400 MHz, (CD₃)₂SO) δ (ppm): 10.41 (br s, 1H), 8.55 (br s, 1H), 4.91-4.80 (m, 1H), 4.76-4.65 (m, 1H), 4.40-4.20 (m, 3H), 3.92 (d, 1H), 3.74-3.64 (m, 1H), 3.55-3.45 (m, 1H), 1.44 (d, 3H), 1.36 (d, 3H), 1.30 (t, 3H).

### Preparation 45

### 1-(6-Fluoropyridin-3-yl)pyrrolidin-2-one (P45)

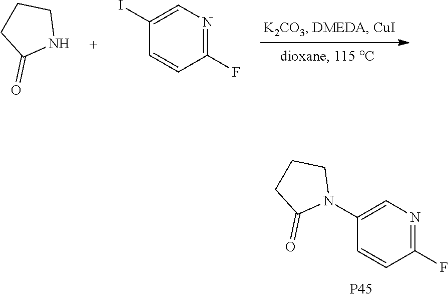 [SMILES: O=C1CCCN1 ; Fc1ccc(I)cn1 ; "P45" ; O=C1CCCN1c1ccc(F)nc1]

### Step 1. Preparation of 1-(6-fluoropyridin-3-yl)pyrrolidin-2-one (P45)

A reaction mixture of 2-fluoro-5-iodopyridine (CAS: 171197-80-1; 2.00 g, 8.97 mmol), pyrrolidin-2-one (CAS: 616-45-5; 0.763 g, 8.97 mmol), K₂CO₃ (3.72 g, 26.9 mmol), DMEDA (0.158 g, 1.79 mmol) and CuI (0.342 g, 1.79 mmol) in dioxane (40 mL) was heated to 115 °C for 16 h under nitrogen gas. The suspension was filtered, and the filtrate was concentrated in vacuo. The residue was purified by column chromatography (silica gel, 0-60% EtOAc: PE) to provide P45 (1.40 g, 86.6% yield) as a light-yellow solid. (LC/MS) m/z (M+H)⁺=181.0; ¹H NMR (400 MHz, (CD₃)₂SO) δ (ppm): 8.46-8.43 (m, 1H), 8.36-8.29 (m, 1H), 7.22 (dd, 1H), 3.89-3.83 (m, 2H), 2.54-2.51 (m, 1H), 2.50-2.48 (m, 1H), 2.14-2.04 (m, 2H).

### Preparation 46

### Ethyl (R)-1-ethyl-6-methyl-4,5,6,7-tetrahydro-1H-pyrazolo[4,3-c]pyridine-3-carboxylate hydrochloride (P46)

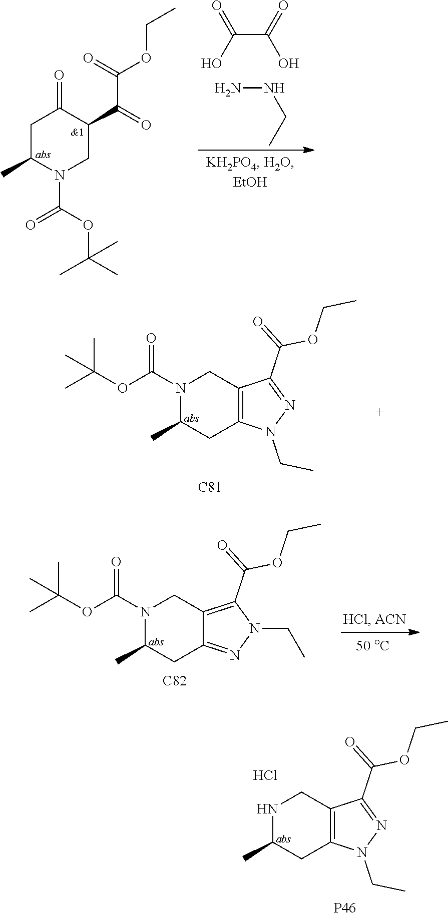

### Step 1. Preparation of 5-(tert-butyl) 3-ethyl (R)-1-ethyl-6-methyl-1,4,6,7-tetrahydro-5H-pyrazolo[4,3-c]pyridine-3,5-dicarboxylate (C81) and 5-(tert-butyl) 3-ethyl (R)-2-ethyl-6-methyl-2,4,6,7-tetrahydro-5H-pyrazolo[4,3-c]pyridine-3,5-dicarboxylate (C82)

A reaction mixture of H₂O (8.8 mL), EtOH (8.8 mL), tert-butyl(2S,5RS)-5-(2-ethoxy-2-oxoacetyl)-2-methyl-4-oxopiperidine-1-carboxylate (CAS: 2171296-27-6; 1.65 g, 5.27 mmol), ethylhydrazine oxalate (0.949 g, 6.32 mmol) and KH₂PO₄ (1.00 g, 7.37 mmol) was stirred at room temperature for 2 h. The yellow slurry was checked by LCMS, which showed C81 (LC/MS) m/z (M+H)⁺=338.3 at 1.69 min retention time and C82 (LC/MS) m/z (M+H)⁺=338.3 at 1.94 min retention time. The reaction mixture was diluted with H₂O and extracted with DCM (3×). The combined organic layer was dried with Na₂SO₄ and then purified by column chromatography (silica gel, 0-100% EtOAc: heptane) to provide C81 (1.53 g, 86.0% yield) and C82 (30.0 mg, 1.70% yield).

C81: ¹H NMR (400 MHz, (CD₃)₂SO) b (ppm): 4.88 (d, 1H), 4.78-4.65 (m, 1H), 4.33-4.17 (m, 2H), 4.14-3.94 (m, 3H), 2.85 (dd, 1H), 2.68-2.60 (m, 1H), 1.42 (s, 9H), 1.33-1.26 (m, 6H), 1.03 (d, 3H).

C82: ¹H NMR (400 MHz, (CD₃)₂SO) b (ppm): 4.91 (d, 1H), 4.78-4.63 (m, 1H), 4.54-4.36 (m, 2H), 4.35-4.15 (m, 2H), 4.09-3.96 (m, 2H), 3.38-3.26 (m, 1H), 2.82-2.65 (m, 2H), 2.56-2.51 (m, 1H), 2.47-2.36 (m, 1H), 2.28-2.12 (m, 2H), 1.35-1.27 (m, 6H), 1.10-1.06 (m, 3H), 1.04-0.99 (m, 3H).

### Step 2. Preparation of ethyl (R)-1-ethyl-6-methyl-4,5,6,7-tetrahydro-1H-pyrazolo[4,3-c]pyridine-3-carboxylate hydrochloride (P46)

To a solution of C81 (1.00 g, 2.96 mmol) in ACN (10 mL) was added 12.1M HCl (0.216 g, 5.93 mmol). The reaction mixture was stirred at 50 °C for 25 h and 15 min, then concentrated in vacuo. The residue was diluted with ACN and then concentrated in vacuo. The residue was diluted with DCM and concentrated in vacuo (2×) to provide P46 (0.730 g, crude) as a yellow foam. The yellow foam was used directly in the next step without further purification. (LC/MS) m/z (M+H)⁺=238.2. ¹H NMR (600 MHz, (CD₃)₂SO) δ (ppm): 9.33-9.24 (m, 1H), 9.07-8.93 (m, 1H), 4.38-4.10 (m, 6H), 3.48-3.39 (m, 1H), 3.16 (dd, 1H), 2.71 (dd, 1H), 1.41-1.37 (m, 3H), 1.31 (m, 6H).

### Preparation 47

### Methyl 3-methyl-5,6,7,8-tetrahydroimidazo[1,5-a]pyrazine-1-carboxylate (47)

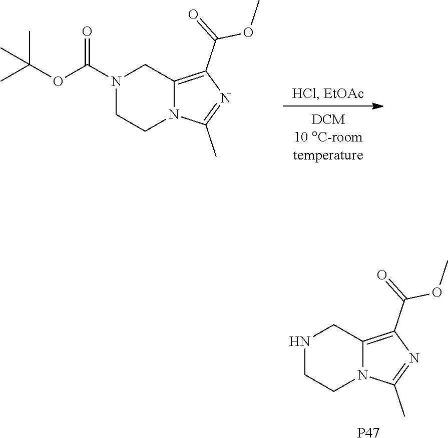 [SMILES: COC(=O)c1nc(C)n2c1CN(C(=O)OC(C)(C)C)CC2 ; "P47" ; COC(=O)c1nc(C)n2c1CNCC2]

### Step 1. Preparation of methyl 3-methyl-5,6,7,8-tetrahydroimidazo[1,5-a]pyrazine-1-carboxylate hydrochloride (P47)

To a solution of 7-(tert-butyl) 1-methyl 3-methyl-5,6-dihydroimidazo[1,5-a]pyrazine-1,7(8H)-dicarboxylate (CAS: 1359655-89-2, 0.190 g, 0.643 mmol) in DCM (5.0 mL) was added 2M HCl in EtOAc (5.0 mL) at 10 °C. The reaction mixture was stirred at room temperature for 16 h. The reaction mixture became a white suspension concentrated in vacuo to provide the P47 (0.149 g, crude) as a white solid. This was used in the next step without further purification. ¹H NMR (400 MHz, CD₃OD) δ (ppm): 4.80 (s, 2H), 4.51 (t, 2H), 4.00 (s, 3H), 3.84 (t, 2H), 2.71 (s, 3H).

### Preparation 48

### Ethyl 3-isopropyl-5,6,7,8-tetrahydroimidazo[1,5-a]pyrazine-1-carboxylate hydrochloride (P48)

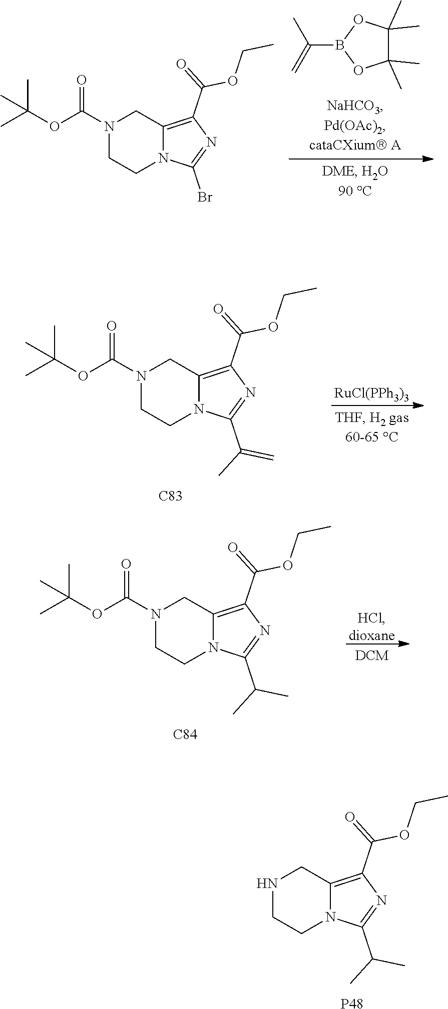

### Step 1. Preparation of 7-(tert-butyl) 1-ethyl 3-(prop-1-en-2-yl)-5,6-dihydroimidazo[1,5-a]pyrazine-1,7(8H)-dicarboxylate (C83)

A solution of 7-(tert-butyl) 1-ethyl 3-bromo-5,6-dihydroimidazo[1,5-a]pyrazine-1,7(8H)-dicarboxylate (CAS: 2108354-93-2; 1.00 g, 2.67 mmol), 4,4,5,5-tetramethyl-2-(prop-1-en-2-yl)-1,3,2-dioxaborolane (CAS: 126726-62-3, 0.808 g, 4.81 mmol), NaHCO₃ (0.673 g, 8.02 mmol), cataCXium® A (0.192 g, 0.534 mmol) and Pd(OAc)₂ (60.0 mg, 0.267 mmol) in (4:1) DME: H₂O (16 mL: 4 mL) was degassed with nitrogen gas for 5 min. The reaction solution was stirred at 90 °C for 16 h and then concentrated in vacuo. The residue was dissolved in DCM (50 mL) and then extracted with EtOAc (3×50 mL). The combined organic layer was dried with Na₂SO₄, filtered, and then concentrated in vacuo to give a residue. The residue was purified by column chromatography (silica gel, 0-20% EtOAc: PE) to provide C83 (0.856 g, 95.5% yield) as a light-yellow solid. (LC/MS) m/z (M+H)⁺=336.2.

### Step 2. Preparation of 7-(tert-butyl) 1-ethyl 3-isopropyl-5,6-dihydroimidazo[1,5-a]pyrazine-1,7(8H)-dicarboxylate (C84)

To the suspension of C83 (0.86 g, 2.5 mmol) in THF (20 mL) was added RuCI(PPh₃)₃(0.24 g, 0.25 mmol). The reaction mixture was degassed with hydrogen gas and then stirred under a hydrogen atmosphere (50 psi) from 60-65 °C for 48 h. The suspension was concentrated in vacuo, and then the residue was purified by column chromatography (silica gel, 0-20% THF: PE) to provide C84 (0.67 g, 77% yield) as a brown solid. (LC/MS) m/z (M+H)⁺=338.3. ¹H NMR (400 MHz, CDCl₃) δ (ppm): 4.87 (s, 2H), 4.36 (q, 2H), 3.92 (t, 2H), 3.82 (t, 2H), 3.03-2.89 (m, 1H), 1.50 (s, 9H), 1.43-1.32 (m, 9H).

### Step 3. Preparation of ethyl 3-isopropyl-5,6,7,8-tetrahydroimidazo[1,5-a]pyrazine-1-carboxylate hydrochloride (P48)

To a solution of C84 (0.67 g, 2.0 mmol) in DCM (10 mL) was added 2M HCl in dioxane (20 mL). The reaction solution was stirred at room temperature for 2 h then was concentrated in vacuo and was dried further by high vacuo to provide P48 (0.54 g, crude) as a solid. The solid was used directly in the next step without further purification.

### Preparation 49

### 5,7-Di-tert-butyl-2-(oxetan-3-yloxy)-3-phenyl-2,3-dihydrobenzo[d]oxazole (P49)

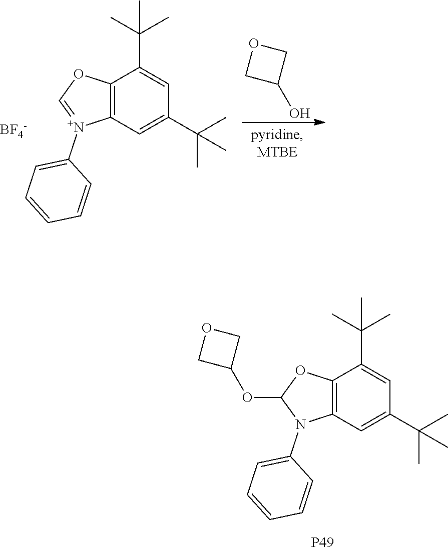 [SMILES: "BF4-" ; CC(C)(C)c1cc(C(C)(C)C)c2oc[n+](-c3ccccc3)c2c1 ; OC1COC1 ; "P49" ; CC(C)(C)c1cc2c(c(C(C)(C)C)c1)OC(OC1COC1)N2c1ccccc1]

### Step 1. Preparation of 5,7-di-tert-butyl-2-(oxetan-3-yloxy)-3-phenyl-2,3-dihydrobenzo[d]oxazole (P49)

A reaction mixture of 3-hydroxyoxetane (CAS: 7748-36-9; 41 mg, 0.55 mmol) and deoxazole (CAS: 1207294-92-5; 0.22 g, 0.55 mmol) in MTBE (3 mL) was degassed with nitrogen gas (2×). The suspension was stirred at room temperature for 5 min before adding pyridine (43 mg, 0.55 mmol). The reaction mixture was stirred for 30 min to provide a solution of P49 (0.21 g, crude). The solution was used directly in the next step without further purification.

### Preparation 50

### rac-Methyl (4R)-1-isopropyl-4-methyl-3a,4,5,6,7,7a-hexahydro-1H-pyrazolo[4,3-c]pyridine-3-carboxylate (P50)

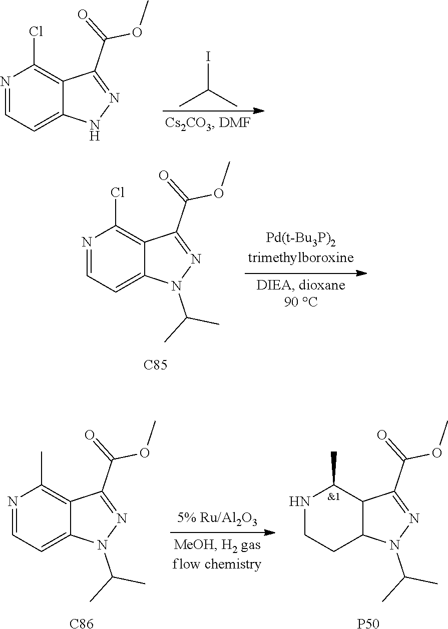

### Step 1. Preparation of methyl 4-chloro-1-isopropyl-1H-pyrazolo[4,3-c]pyridine-3-carboxylate (C85)

To a solution of methyl 4-chloro-1H-pyrazolo[4,3-c]pyridine-3-carboxylate (CAS: 1658466-48-8; 6.00 g, 28.3 mmol) in DMF (120 mL) was added Cs₂CO₃ (18.5 g, 56.7 mmol) and 2-iodopropane (CAS: 75-30-9; 7.23 g, 42.5 mmol). The reaction mixture was stirred at room temperature for 3 h, diluted with EtOAc (100 mL), and washed with brine (3×300 mL). The organic layer was dried with Na₂SO₄ and then concentrated in vacuo. The yellow oil was purified by column chromatography (silica gel, 0-37% EtOAc:hexane) to provide C85 (3.30 g, 45.9% yield). (LC/MS) m/z (M+H)⁺=254.0. ¹H NMR (400 MHz, (CD₃)₂SO) δ (ppm): 8.26 (d, 1H), 7.93 (d, 1H), 5.22-5.07 (m, 1H), 3.93 (s, 3H), 1.49 (d, 6H).

### Step 2. Preparation of methyl 1-isopropyl-4-methyl-1H-pyrazolo[4,3-c]pyridine-3-carboxylate (C86)

To a solution of C85 (2.10 g, 8.28 mmol), trimethylboroxine (CAS: 823-96-1; 8.31 g, 33.1 mmol) in dioxane (60 mL) was added DIEA (4.28 g, 33.1 mmol) and Pd(t-Bu₃P)₂ (0.635 g, 1.24 mmol). The reaction mixture was degassed with nitrogen gas and then stirred at 90 °C for 12 h. The 5 suspension was diluted with EtOAc (100 mL), washed with brine (2×150 mL), dried with Na₂SO₄, filtered, and concentrated in vacuo. The residue was purified by column chromatography (silica gel, 0-50% EtOAc:hexane) to provide C86 (2.10 g, crude) as an orange gum. The gum was used directly in the next step without further purification. (LC/MS) m/z (M+H)⁺=234.0. ¹H NMR (400 MHz, CDCl₃) δ (ppm): 8.33 (d, 1H), 7.24 (d, 1H), 4.96-4.83 (m, 1H), 4.03 (s, 3H), 3.06 (s, 3H), 1.65-1.61 (m, 6H).

### Step 3. Preparation of rac-methyl (4R)-1-isopropyl-4-methyl-3a,4,5,6,7,7a-hexahydro-1H-pyrazolo[4,3-c]pyridine-3-carboxylate (P50)

The reaction was carried out via flow chemistry. C86 (1.80 g, 7.72 mmol) was dissolved in MeOH (40 mL). The solution was pumped with a flow rate of 0.3 mL/min, and the H₂ had a 30 mL/min rate. The solution passed through the fixed bed (¼″ mm) packed with granular catalyst 5% Ru/Al20₃ with a 5.0 mL volume and was hydrogenated at 60 °C. via flow at 2.0 MPa H₂. The reaction mixture was collected from the reactor output to provide P50 (1.85 g, crude) as a black oil. The oil was used directly in the next step without further purification. (LC/MS) m/z (M+H)⁺=238.2. ¹H NMR (400 MHz, (CD₃)₂SO) δ (ppm): 4.61-4.42 (m, 2H), 4.24-4.01 (m, 1H), 3.86-3.75 (m, 3H), 3.04-2.77 (m, 4H), 1.49 (d, 3H), 1.38 (dd, 6H).

### Preparation 51

### rac-Methyl (4R)-4-ethyl-1-isopropyl-3a,4,5,6,7,7a-hexahydro-1H-pyrazolo[4,3-c]pyridine-3-carboxylate (P51)

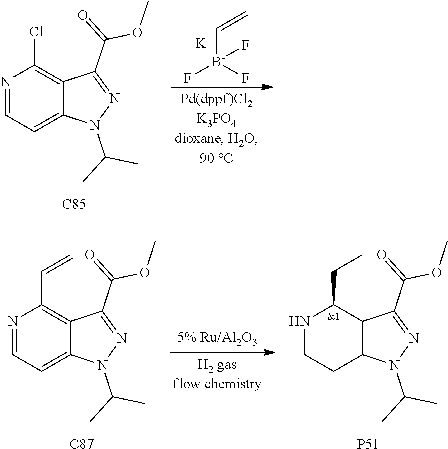

### Step 1. Preparation of methyl 1-isopropyl-4-vinyl-1H-pyrazolo[4,3-c]pyridine-3-carboxylate (C87)

To a reaction mixture of C85 (1.00 g, 3.94 mmol), potassium vinyltrifluoroborate (CAS: 13682-77-4; 0.792 g, 5.91 mmol) and K₃PO₄ (2.09 g, 9.85 mmol) in dioxane (10.0 mL) and H₂O (3.5 mL) was added Pd(dppf)Cl₂ (0.288 g, 0.394 mmol). The suspension was degassed with nitrogen gas for 3 min and then stirred at 90 °C for 2 h. The reaction mixture was filtered, and then the filter cake was washed with EtOAc (50 mL). The filtrate was concentrated in vacuo. The yellow gum was purified by column chromatography (silica gel, 0-40% EtOAc:hexane) to provide C87 (0.700 g, 72.4% yield) as a light yellow solid. (LC/MS) m/z (M+H)⁺=246.1. ¹H NMR (400 MHz, (CD₃)₂SO) δ (ppm): 8.45 (d, 1H), 8.07 (dd, 1H), 7.79 (d, 1H), 6.51 (dd, 1H), 5.59 (dd, 1H), 5.19-5.08 (m, 1H), 3.93 (s, 3H), 1.50 (d, 6H).

### Step 2. Preparation of rac-methyl (4R)-4-ethyl-1-isopropyl-3a,4,5,6,7,7a-hexahydro-1H-pyrazolo[4,3-c]pyridine-3-carboxylate (P51)

The reaction was carried out via flow chemistry. C87 (0.300 g, 1.22 mmol) was dissolved in MeOH (20 mL). The solution was pumped with a flow rate of 0.3 mL/min, and the H₂ had a 30 mL/min rate. The solution passed through the fixed bed (¼″ mm) packed with granular catalyst 5% Ru/Al₂O₃ with a 5.0 mL volume and was hydrogenated at 60 °C. via flow at 2.0 MPa H₂. The reaction mixture was collected from the reactor output to provide P51 (0.280 g, crude) as a brown oil. The oil was used directly in the next step without further purification. (LC/MS) m/z (M+H)⁺=252.0. ¹H NMR (400 MHz, (CD₃)₂SO) δ (ppm): 9.75 (s, 1H), 9.23 (s, 1H), 4.59-4.50 (m, 1H), 3.78 (s, 3H), 3.07-2.89 (m, 2H), 2.45-2.41 (m, 2H), 2.00-1.75 (m, 2H), 1.35 (dd, 6H), 1.02 (t, 3H).

### Preparation 52

### rac-Ethyl (6R,7R)-1-isopropyl-6,7-dimethyl-4,5,6,7-tetrahydro-1H-pyrazolo[4,3-c]pyridine-3-carboxylate (P52)

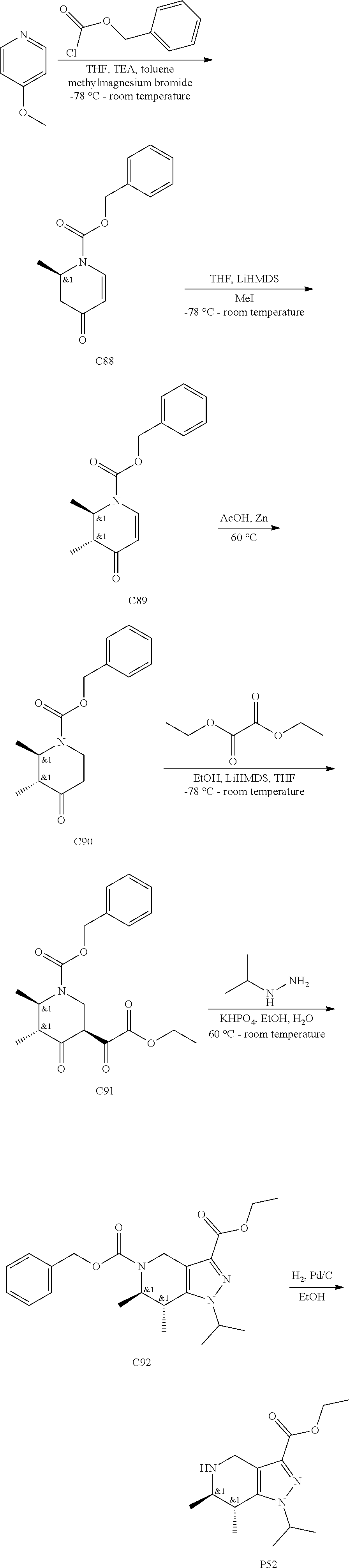

### Step 1. Preparation of rac-benzyl (R)-2-methyl-4-oxo-3,4-dihydropyridine-1(2H)-carboxylate (C88)

To a round bottom flask was added 4-methoxypyridine (CAS: 620-08-6; 32.6 mL), THF (641.4 mL), and TEA (4.5 mL) sequentially. The solution was cooled to −78 °C, and then 3M benzyl chloroformate in toluene (109.0 mL) was added dropwise to give a white slurry. 3.4M methylmagnesium bromide solution (55.6 mL) dropwise was added to the suspension. The reaction mixture was stirred at −78 °C and slowly warmed to room temperature over 24 h. The reaction mixture was cooled to 0 °C and quenched via a portion-wise addition of 1 M aqueous HCl. The aqueous mixture was extracted with DCM (3×). The organic layer was combined, then dried with Na₂SO₄ and purified by column chromatography (silica gel, 0-50% EtOAc: heptane) to provide C88 (51.5 g, 65.5% yield) as a clear oil. (LC/MS) m/z (M+H)⁺=246.4. ¹H NMR (400 MHz, (CD₃)₂SO) δ (ppm): 7.78 (dd, 1H), 7.48-7.28 (m, 5H), 5.33-5.21 (m, 3H), 4.68-4.56 (m, 1H), 2.89 (dd, 1H), 2.24-2.14 (m, 1H), 1.15 (d, 3H).

### Step 2. Preparation of rac-benzyl(2R,3R)-2,3-dimethyl-4-oxo-3,4-dihydropyridine-1(2H)-carboxylate (C89)

A reaction mixture of C88 (10.0 g, 40.8 mmol) in THF (102 mL) was cooled to −78 °C, then 1 M LiHMDS in THF (42.8 mL) was added dropwise and stirred at −78 °C for 10 min. After stirring for a few min, Mel (3.1 mL) was added dropwise at −78 °C to the reaction mixture and then warmed to room temperature over 30 min. The suspension was stirred at room temperature for 9 h and then quenched with brine. The pH of the aqueous layer was adjusted to pH=1 using 12M aqueous HCl. The reaction mixture was extracted with EtOAc (2×), and the organic layers were combined and then dried with Na₂SO₄. The mixture was purified by column chromatography (silica gel, 0-40% MTBE: heptane) to provide C89 (7.80 g, 73.6% yield) as a yellow oil. ¹H NMR (400 MHz, CD₃OD) δ (ppm): 7.84 (dd, 1H), 7.47-7.32 (m, 5H), 5.36-5.20 (m, 3H), 4.40 (q, 1H), 2.27 (q, 1H), 1.21 (d, 3H), 1.15 (d, 3H).

### Step 3. Preparation of rac-benzyl(2R,3R)-2,3-dimethyl-4-oxopiperidine-1-carboxylate (C90)

To a round bottom flask was added C89 (12.8 g, 49.4 mmol), AcOH (82.3 mL), and Zn (12.9 g, 197 mmol) sequentially, then stirred at 60 °C for 5 h. The reaction mixture was concentrated in vacuo, then saturated NaHCO₃ and extracted with DCM (3×). The combined organic layer was purified by column chromatography (silica gel, 0-50% EtOAc: heptane) to provide C90 (10.1 g, 78.3% yield) as a clear oil. ¹H NMR (400 MHz, (CD₃)₂SO) δ (ppm): 7.44-7.28 (m, 5H), 5.17-5.06 (m, 2H), 4.13 (dd, 1H), 4.07-3.96 (m, 1H), 3.53-3.41 (m, 1H), 2.57-2.44 (m, 2H), 2.33-2.23 (m, 1H), 1.21 (d, 3H), 1.05 (d, 3H).

### Step 4. Preparation of rac-benzyl(2R,3R,5R)-5-(2-ethoxy-2-oxoacetyl)-2,3-dimethyl-4-oxopiperidine-1-carboxylate (C91)

A reaction mixture of C90 (0.430 g, 1.65 mmol), EtOH (3.29 mL), and diethyl oxalate (0.24 mL) was cooled to −78 °C, then 1 M LiHMDS in THF (1.89 mL) was added. The suspension was stirred at room temperature for 17 h and then quenched with a mixture of 1 M aqueous HCl, brine, and DCM. The aqueous layer was extracted with (2:1, MTBE: DCM, 2×). The organic layers were combined and then dried with Na₂SO₄ and concentrated in vacuo to provide C91 (0.590 g, crude) as a yellow oil. The oil was used directly in the next step without further purification.

### Step 5. Preparation of rac-5-benzyl 3-ethyl(6R,7R)-1-isopropyl-6,7-dimethyl-1,4,6,7-tetrahydro-5H-pyrazolo[4,3-c]pyridine-3,5-dicarboxylate (C92)

A reaction mixture of C91 (0.590 g, 1.63 mmol), KHPO₄ (0.311 g, 2.29 mmol) and isopropylhydrazine HCl (217 mg, 1.96 mmol) in EtOH (2.7 mL) and H₂O (2.7 mL) was stirred at 60 °C for 2.5 h then stirred at room temperature for 24 h. The suspension was diluted with H₂O and then extracted with DCM (3×). The organic layers were combined and then purified with column chromatography (silica gel, 0-60% EtOAc: heptane) to provide C92 (0.322 g, 49.4% yield) as a clear oil.

### Step 6. Preparation of rac-ethyl (6R,7R)-1-isopropyl-6,7-dimethyl-4,5,6,7-tetrahydro-1H-pyrazolo[4,3-c]pyridine-3-carboxylate (P52)

In a reactor was added Pd/C (10 wt %, 0.213 g, 2.00 mmol) in EtOH (1 mL), then added solution of C92 (0.800 g, 2.00 mmol) in EtOH (5 mL). The suspension was degassed with hydrogen gas and then stirred under a hydrogen atmosphere (120 psi) at room temperature for 1 h. The reaction mixture was filtered through Celite and then washed with tert-amyl alcohol. The filtrate was concentrated in vacuo to provide P52 (0.531 g, crude) as a yellow oil. The oil was used directly in the next step without further purification. ¹H NMR (400 MHz, (CD₃)₂SO) δ (ppm): 4.57-4.46 (m, 1H), 4.23 (q, 2H), 3.82-3.68 (m, 2H), 2.84-2.74 (m, 1H), 2.62-2.55 (m, 1H), 1.43 (d, 3H), 1.39-1.31 (m, 6H), 1.22-1.18 (m, 3H), 1.05-1.01 (m, 3H).

### Preparation 53

### rac-Ethyl (R)-3-isopropyl-5-methyl-5,6,7,8-tetrahydroimidazo[1,5-a]pyrazine-1-carboxylate hydrochloride (P53)

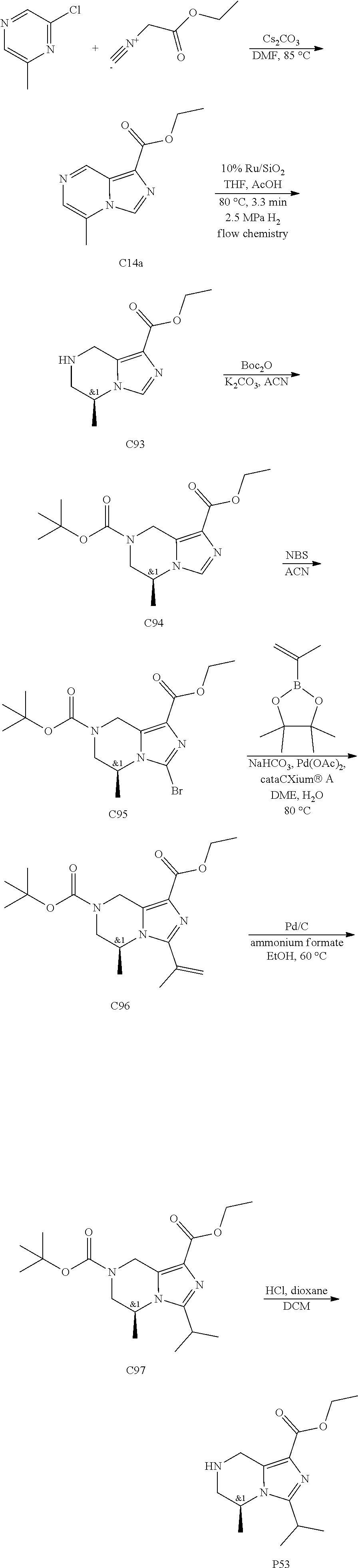

### Step 1. Preparation of ethyl 5-methylimidazo[1,5-a]pyrazine-1-carboxylate (C14a)

To a mixture of 2-chloro-6-methylpyrazine (CAS: 38557-71-0, 10.0 g, 77.8 mmol) in DMF (150.0 mL) was added Cs₂CO₃ (38.0 g, 117 mmol) and ethyl 2-isocyanoacetate (CAS: 2999-46-4, 17.8 g, 156 mmol) at 15 °C. The reaction was heated to 85 °C for 16 h, then diluted with EtOAc (300 mL). The light brown reaction mixture was filtered, and the filtrate was concentrated in vacuo. The brown residue was diluted with (1:1, EtOAc: THF) and filtered. The filter cake was dissolved in (1:1, H₂O: EtOAc) and then extracted with EtOAc (150 mL). The combined organic phase was dried with Na₂SO₄ and concentrated in vacuo. The residue was purified by column chromatography (silica gel, 0-100% EtOAC: PE) to provide C14a (7.71 g, 48.3% yield) as a light-brown solid. (LC/MS) m/z (M+H)⁺=206.1. ¹H NMR (400 MHz, CDCl₃) δ (ppm): 9.54 (s, 1H), 8.16 (s, 1H), 7.66 (s, 1H), 4.52 (q, 2H), 2.65 (s, 3H), 1.48 (t, 3H).

### Step 2. Preparation of rac-ethyl (R)-5-methyl-5,6,7,8-tetrahydroimidazo[1,5-a]pyrazine-1-carboxylate acetate (C93)

The reaction was carried out via flow chemistry. C14a (7.71 g, 37.6 mmol) was dissolved in THF (155 mL) and AcOH (40 mL) and then was pumped with a flow rate of 0.3 mL/min, and the H₂ had a rate of 30 mL/min. The solution passed through the fixed bed ((¼″) mm) packed with granular catalyst 10% Ru/SiO₂ (2.80 g, 2.76 mmol) with a 5.0 mL volume and was hydrogenated for 3.3 min at 80 °C. via flow at 2.5 MPa H₂. The flow rate of H₂ was 100 mL/min. The reaction mixture was collected after 10 min and then concentrated in vacuo to remove THF. The suspension was stirred for 1-2 h and then filtered. The filtrate was concentrated in vacuo to provide C93 (5.50 g, crude) as a light-brown oil. The oil was used directly in the next step without further purification. (LC/MS) m/z (M+H)⁺=210.1.

### Step 3. Preparation of rac-7-(tert-butyl) 1-ethyl (R)-5-methyl-5,6-dihydroimidazo[1,5-a]pyrazine-1,7(8H)-dicarboxylate (C94)

To a solution of C93 (7.50 g, 19.3 mmol) in ACN (100 mL) was added Boc₂O (6.31 g, 28.9 mmol) and K₂CO₃ (10.7 g, 77.1 mmol) at room temperature. The reaction was stirred at room temperature for 16 h and then diluted with EtOAc (100 mL). The suspension was filtered then the filtrate was concentrated in vacuo. The residue was purified by column chromatography (silica gel; 0-100% EtOAc: PE) to provide C94 (5.30 g, 93.1% yield) as an off-white solid. (LC/MS) m/z (M+H)⁺=310.2. ¹H NMR (400 MHz, CDCl₃) δ (ppm): 7.53 (s, 1H), 4.96-4.78 (m, 2H), 4.41-4.22 (m, 3H), 3.95-3.90 (m, 1H), 3.48-3.43 (m, 1H), 1.55-1.49 (m, 12H), 1.39 (t, 3H).

### Step 4. Preparation of rac-7-(tert-butyl) 1-ethyl (R)-3-bromo-5-methyl-5,6-dihydroimidazo[1,5-a]pyrazine-1,7(8H)-dicarboxylate (C95)

To a solution of C94 (3.20 g, 10.8 mmol) in ACN (30.0 mL) was added a solution of NBS (2.89 g, 16.3 mmol) in ACN (20.0 mL). The reaction mixture was stirred at room temperature for 16 h and then concentrated in vacuo. The residue was purified by column chromatography (silica gel, 0-40% EtOAc: PE) to provide C95 (2.57 g, 64.0% yield) as a white solid. (LC/MS) m/z (M+2H)⁺=390.0. ¹H NMR (400 MHz, CDCl₃) δ (ppm): 5.59-5.25 (m, 1H), 4.51-4.25 (m, 5H), 3.40-3.12 (m, 1H), 1.51 (s, 9H), 1.45-1.29 (m, 6H).

### Step 5. Preparation of rac-7-(tert-butyl) 1-ethyl (R)-3-isopropyl-5-methyl-5,6-dihydroimidazo[1,5-a]pyrazine-1,7(8H)-dicarboxylate (C96)

A solution of C95 (1.00 g, 2.58 mmol), 4,4,5,5-tetramethyl-2-(prop-1-en-2-yl)-1,3,2-dioxaborolane (CAS: 126726-62-3, 0.779 g, 4.64 mmol), NaHCO₃ (0.649 g, 7.73 mmol), cataCXium® A (0.185 g, 0.515 mmol) and Pd(OAc)₂ (57.8 mg, 0.258 mmol) in (4:1) DME: H₂O (16 mL: 4 mL) was degassed with nitrogen gas for 5 min. The reaction solution was stirred at 80 °C for 15 h, then filtered and concentrated in vacuo. The residue was purified by column chromatography (silica gel, 0-35% EtOAc: PE) to provide C96 (0.810 g, 90.0% yield) as a gum. (LC/MS) m/z (M+H)⁺=350.2. ¹H NMR (400 MHz, (CD₃)₂SO) δ (ppm): 5.49-5.38 (m, 2H), 5.26-5.05 (m, 1H), 4.79-4.65 (m, 1H), 4.58-4.36 (m, 1H), 4.33-3.97 (m, 4H), 2.10-2.06 (m, 3H), 1.43 (s, 9H), 1.31-1.24 (m, 3H), 1.23-1.16 (m, 3H).

### Step 6. Preparation of rac-7-(tert-butyl) 1-ethyl (R)-5-methyl-3-(prop-1-en-2-yl)-5,6-dihydroimidazo[1,5-a]pyrazine-1,7(8H)-dicarboxylate (C97)

To a solution of C96 (0.810 g, 2.32 mmol) in EtOH were added ammonium formate (2.19 g, 34.8 mmol) and Pd/C (0.370 g, 0.348 mmol). The reaction mixture was stirred at 60 °C under argon gas for 1 h and then filtered through celite. The filter cake was washed with EtOH (3×30 mL). The filtrate was concentrated in vacuo and then dissolved in DCM (50 mL). The solution was washed with brine (3×50 mL). The organic layer was concentrated in vacuo to provide C97 (0.715 g, crude). The crude was used directly into the next step without further purification. (LC/MS) m/z (M+H)⁺=352.2.

### Step 7. Preparation of rac-ethyl (R)-3-isopropyl-5-methyl-5,6,7,8-tetrahydroimidazo[1,5-a]pyrazine-1-carboxylate hydrochloride (P53)

To a solution of C97 (0.715 g, 2.03 mmol) in DCM (10 mL) was added 2M HCl in dioxane (20 mL). The reaction mixture was stirred for 2 h at room temperature, then concentrated in vacuo and lyophilized to provide P53 (0.710 g, crude) as a gum. The gum was used directly in the next step without further purification. (LC/MS) m/z (M+H)⁺=252.2.

### Preparation D1 and P54

### tert-butyl (4-(3-Methyl-1,2,4-triazin-6-yl)benzyl)carbamate (D1) and (4-(3-Methyl-1,2,4-triazin-6-yl)phenyl)methanamine (P54)

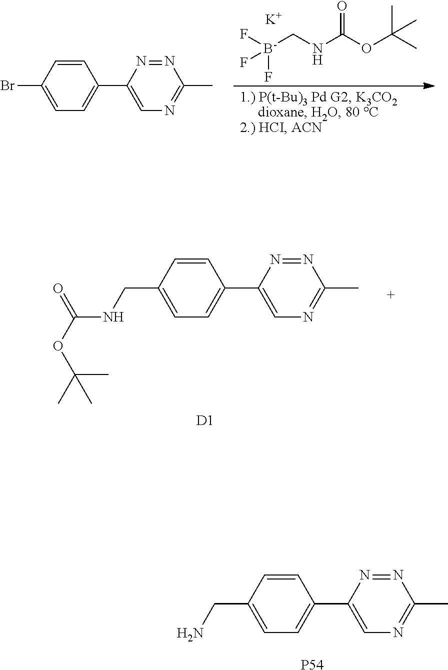

### Step 1. Preparation of tert-butyl (4-(3-methyl-1,2,4-triazin-6-yl)benzyl)carbamate (D1) and (4-(3-methyl-1,2,4-triazin-6-yl)phenyl)methanamine (P54)

A reaction mixture of 6-(4-bromophenyl)-3-methyl-1,2,4-triazine (CAS: 64493-86-3; 0.25 g, 1.0 mmol), potassium [[(tert-butoxycarbonyl)amino]methyl]trifluoroborate (0.28 g, 1.2 mmol), K₃CO₂ (0.41 g, 3.0 mmol), and P(t-Bu)₃ Pd G2 (26 mg, 0.050 mmol) was degassed with nitrogen gas then dioxane (3.0 mL) and H₂O (0.6 mL) were added. The reaction mixture was stirred at 80 °C for 40 min then diluted with EtOAc/H₂O. The organic layer was separated then concentrated in vacuo. The residue was suspended in DCM and filtered. The filtrate was purified by column chromatography (silica gel; 0-80% EtOAc/heptane) to provide D1 (0.12 g, 0.40 mmol) as a yellow solid. D1: (LC/MS) m/z (M+H)⁺=301.3. ¹H NMR (600 MHz, (CD₃)₂SO) b (ppm): 9.29 (s, 1H), 8.14 (d, 2H), 7.49 (t, 1H), 7.44 (d, 2H), 4.21 (d, 2H), 2.81 (s, 3H), 1.42-1.37 (m, 9H).

To a solution of D1 (0.12 g, 0.40 mmol) in ACN (2 mL) was added 12.1 M HCl (0.11 g, 0.25 mL, 3.0 mmol) then stirred at room temperature for 30 min. The reaction mixture was concentrated in vacuo to provide P54 (0.20 g, crude) as a yellow solid. The solid was used directly in the next step without further purification.

### Preparation P55

### (5,7-Dihydro-6H-pyrrolo[3,4-b]pyridin-6-yl)(6-fluoropyridin-3-yl)methanone (P55)

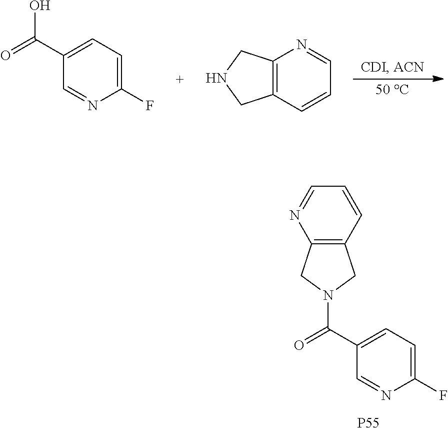 [SMILES: O=C(O)c1ccc(F)nc1 ; c1cnc2c(c1)CNC2 ; "P55" ; O=C(c1ccc(F)nc1)N1Cc2cccnc2C1]

### Step 1. Preparation of (5,7-dihydro-6H-pyrrolo[3,4-b]pyridin-6-yl)(6-fluoropyridin-3-yl)methanone (P55)

A reaction mixture of 3-carboxy-6-fluoropyridine (CAS: 403-45-2; 897 mg, 6.36 mmol) and CDI (1.24 g, 7.63 mmol) in ACN (35 mL) was stirred at room temperature for 1 h then added to 6,7-dihydro-5H-pyrrolo[3,4-b]pyridine (CAS: 147739-88-6; 0.840 g, 6.99 mmol). This reaction mixture was stirred at 50 °C for 18 h then concentrated in vacuo. The residue was suspended in H₂O (15 mL) and stirred at room temperature for 15 min. Precipitation formed and the suspension was filtered with a Buchner funnel. The filter cake was washed with H₂O then dried on the funnel for 10 min to provide (5,7-dihydro-6H-pyrrolo[3,4-b]pyridin-6-yl)(6-fluoropyridin-3-yl)methanone (87.0 mg, 5.61% yield) as a grey solid. The filtrate was extracted with EtOAc (20 mL×3). The combined organic layer was concentrated in vacuo then suspended in ACN (4 mL) and filtered. The filter cake was collected to provide P55 (0.247 g, 15.9% yield) as an off-white solid. The solid was used directly in the next step without further purification.

### Preparation P1A

### rac-(R)-5-(5-(Dimethylcarbamoyl)pyridin-2-yl)-1-ethyl-7,7-dimethyl-4,5,6,7-tetrahydro-1H-indazole-3-carboxylic acid (P1A)

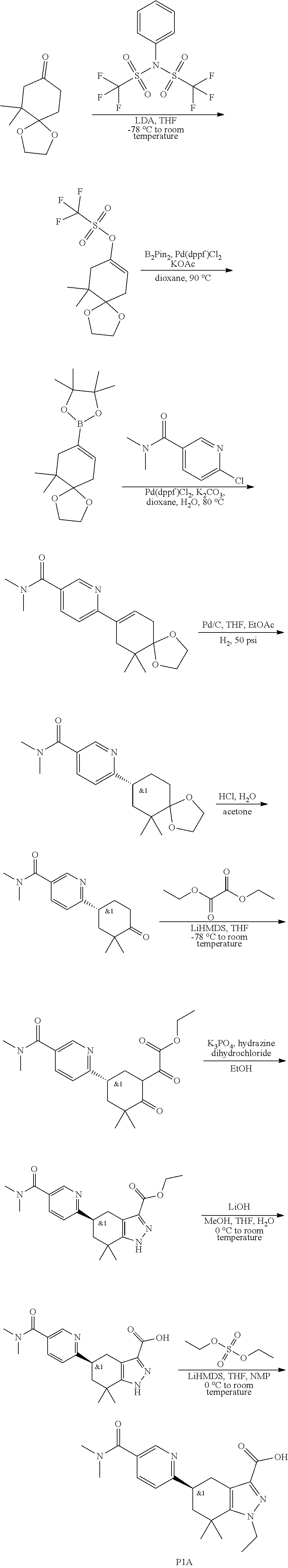

### Step 1. Preparation of 10,10-dimethyl-1,4-dioxaspiro[4.5]dec-7-en-8-yl trifluoromethanesulfonate

To a solution of 6,6-dimethyl-1,4-dioxaspiro[4.5]decan-8-one (CAS: 131206-75-2; 3.29 g, 17.8 mmol) in THF (71 mL) at −78 °C. was slowly added 2M LDA solution (2.29 g, 11 mL, 21.4 mmol). After stirring at −78 °C for approximately 1 h, a solution of 1,1,1-trifluoro-N-phenyl-N-((trifluoromethyl)sulfonyl)methanesulfonamide (7.01 g, 19.6 mmol) in THF (16 mL) was slowly added. The reaction mixture was allowed to warm to room temperature and stirred overnight. The reaction mixture was diluted with EtOAc then washed with saturated aqueous NH₄Cl solution (2×), dried over Na₂SO₄, filtered, and concentrated in vacuo. The oil was dissolved in heptane and a minimal amount of EtOAc then purified by column chromatography (silica gel; 0-30% EtOAc: heptane) to afford 10,10-dimethyl-1,4-dioxaspiro[4.5]dec-7-en-8-yl trifluoromethanesulfonate (5.52 g, 97.9% yield) as a yellow oil. ¹H NMR (600 MHz, CDCl₃) δ (ppm): 5.63-5.58 (m, 1H), 4.02-3.92 (m, 4H), 2.37-2.32 (m, 4H), 1.05-1.00 (m, 6H) (product peaks only).

### Step 2. Preparation of2-(10,10-dimethyl-1,4-dioxaspiro[4.5]dec-7-en-8-yl)-4,4,5,5-tetramethyl-1,3,2-dioxaborolane

To a round bottom flask was added B₂pin₂ (5.10 g, 20.1 mmol), Pd(dppf)Cl₂ (1.07 g, 1.31 mmol), and KOAc (5.14 g, 52.4 mmol) before the findenser was added. The mixture was subjected to three cycles of evacuation and nitrogen backfilling. Through the septum were added a solution of 10,10-dimethyl-1,4-dioxaspiro[4.5]dec-7-en-8-y1 trifluoromethanesulfonate (5.52 g, 17.5 mmol) in dioxane (87.3 mL). The reaction mixture was heated at 90 °C for 2 h. After cooling, the reaction mixture was diluted with EtOAc then filtered through Celite. The filter cake was washed with H₂O. The filtrate was washed with brine then the organic layer was separated. The organic layer was dried over dried over Na₂SO₄, filtered, and concentrated in vacuo to afford 2-(10,10-dimethyl-1,4-dioxaspiro[4.5]dec-7-en-8-yl)-4,4,5,5-tetramethyl-1,3,2-dioxaborolane (5.13 g, crude) as a dark oil. The oil was used directly in the next step without further purification. ¹H NMR (600 MHz, CDCl₃) δ (ppm): 6.44-6.39 (m, 1H), 4.01-3.89 (m, 4H), 2.33-2.26 (m, 2H), 2.22-2.18 (m, 2H), 1.25-1.24 (m, 12H), 0.97-0.90 (m, 6H).

### Step 3. Preparation of 6-(10,10-dimethyl-1,4-dioxaspiro[4.5]dec-7-en-8-yl)-N,N-dimethylnicotinamide

A mixture of 2-(10,10-dimethyl-1,4-dioxaspiro[4.5]dec-7-en-8-yl)-4,4,5,5-tetramethyl-1,3,2-dioxaborolane (5.13 g, 17.4 mmol), 6-chloro-N,N-dimethylnicotinamide (3.86 g, 20.9 mmol), and Pd(dppf)Cl₂ (712 mg, 0.871 mmol) was degassed (3×) with nitrogen gas before 2M K₂CO₃ in H₂O (7.23 g, 26.2 mL, 52.3 mmol) and dioxane (87.2 mL) were added. The reaction mixture was heated at 80 °C for −72 h then diluted with EtOAc and filtered over Celite. The filtrate was washed with brine then the organic layer was separated. The organic layer was dried over Na₂SO₄, filtered, and concentrated in vacuo. The oil was dissolved in a minimal amount of DCM then purified by column chromatography (silica gel; 0-100% EtOAc: heptane then 10% EtOH: DCM) to provide 6-(10,10-dimethyl-1,4-dioxaspiro[4.5]dec-7-en-8-yl)-N,N-dimethylnicotinamide (2.07 g, 37.5% yield) as a caramel oil. (LC/MS) m/z (M+H)⁺=317.3. ¹H NMR (600 MHz, (CD₃)₂SO) δ (ppm): 8.65-8.62 (m, 1H), 7.74 (dd, 1H), 7.50-7.45 (m, 1H), 6.66-6.61 (m, 1H), 4.09-3.97 (m, 4H), 3.14 (s, 3H), 3.04 (s, 3H), 2.63 (q, 2H), 2.52-2.44 (m, 2H), 1.09 (s, 6H).

### Step 4. Preparation of rac-(R)-6-(6,6-dimethyl-1,4-dioxaspiro[4.5]decan-8-yl)-N,N-dimethylnicotinamide

To a vial containing a solution of 6-(10,10-dimethyl-1,4-dioxaspiro[4.5]dec-7-en-8-yl)-N,N-dimethylnicotinamide (2.07 g, 6.54 mmol) in THF (2 mL) and EtOAc (25 mL) was added 10% Pd/C (50% wet by weight, 522 mg, 491 μmol). The reaction mixture was evacuated and refilled with nitrogen three times, then evacuated and refilled with hydrogen twice, and subsequently placed under 50 psi hydrogen atmosphere with stirring overnight. The reaction mixture was diluted with EtOAc then filtered through Celite. The Celite was washed with EtOAc. The filtrate was concentrated under reduced pressure to afford rac-(R)-6-(6,6-dimethyl-1,4-dioxaspiro[4.5]decan-8-yl)-N,N-dimethylnicotinamide (2.08 g, crude) as a yellow oil. The oil was used directly in the next step without further purification. (LC/MS) m/z (M+H)⁺=319.4. ¹H NMR (600 MHz, CDCl₃) δ (ppm): 8.60 (s, 1H), 7.73 (s, 1H), 7.29-7.26 (m, 1H), 4.02-3.91 (m, 4H), 3.18-2.99 (m, 7H), 1.94-1.81 (m, 4H), 1.72-1.65 (m, 1H), 1.62 (d, 1H), 1.18 (s, 3H), 0.93 (s, 3H).

### Step 5. Preparation of rac-(R)-6-(3,3-dimethyl-4-oxocyclohexyl)-N,N-dimethylnicotinamide

To a solution of rac-(R)-6-(6,6-dimethyl-1,4-dioxaspiro[4.5]decan-8-yl)-N,N-dimethylnicotinamide (2.10 g, 6.60 mmol) in acetone (33.0 mL) at room temperature was added 1M aqueous HCl (721 mg, 19.8 mL, 19.8 mmol). The reaction mixture was stirred at room temperature overnight before an additional portion of 1 M aqueous HCl (0.240 g, 6.60 mL, 6.60 mmol) was added. The reaction mixture was stirred at room temperature for 7 h then quenched by slow addition of saturated aqueous NaHCO₃ until gas evolution ceased and the pH=7. The mixture was concentrated in vacuo then the resulting aqueous layer was extracted with DCM (1×) and EtOAc (1×). The combined organic layers were dried over Na₂SO₄, filtered, and concentrated in vacuo to afford a dark oil. This material was further extracted with EtOAc. The organic layer was washed with brine, dried over Na₂SO₄, and concentrated in vacuo to provide a yellow oil. The yellow oil was dissolved in DCM then purified by column chromatography (silica gel; 5-10% EtOH: DCM) to afford rac-(R)-6-(3,3-dimethyl-4-oxocyclohexyl)-N,N-dimethylnicotinamide (1.36 g, 75.2% yield) as a yellow oil. ¹H NMR (600 MHz, CDCl₃) δ (ppm): 8.56-8.53 (m, 1H), 7.67 (dd, 1H), 7.19-7.16 (m, 1H), 3.41-3.33 (m, 1H), 3.06 (s, 3H), 2.97 (s, 3H), 2.75-2.66 (m, 1H), 2.38-2.32 (m, 1H), 2.24-2.16 (m, 1H), 1.99-1.86 (m, 3H), 1.25 (s, 3H), 1.05 (s, 3H).

### Step 6. Preparation of rac-ethyl 2-((5R)-5-(5-(dimethylcarbamoyl)pyridin-2-yl)-3,3-dimethyl-2-oxocyclohexyl)-2-oxoacetate

To a solution of 6-(3,3-dimethyl-4-oxocyclohexyl)-N,N-dimethylnicotinamide (1.36 g, 4.96 mmol) and diethyl oxalate (1.09 g, 1.02 mL, 7.44 mmol) in THF (49.6 mL) at −78 °C. was slowly added 1M LiHMDS in THF (1.16 g, 6.94 mL, 6.94 mmol). The reaction mixture was stirred at −78 °C. for 10 min, then warm to room temperature and stirred for approximately 1 h. The reaction mixture was then cooled again to −78 °C, after which diethyl oxalate (217 mg, 203 μL, 1.49 mmol) was added, followed by 1 M LiHMDS in THF (249 mg, 1.49 mL, 1.49 mmol). The reaction mixture was warmed to room temperature and stirred for 2 h then diluted with H₂O and extracted with EtOAc (1×) then DCM (1×). The combined organic extracts were dried over Na₂SO₄, filtered, and concentrated in vacuo to afford rac-ethyl 2-((5R)-5-(5-(dimethylcarbamoyl)pyridin-2-yl)-3,3-dimethyl-2-oxocyclohexyl)-2-oxoacetate (1.86 g, crude) as an oil. The oil was used directly into the next step without further purification. (LC/MS) m/z (M+H)⁺=375.4.

### Step 7. Preparation of rac-ethyl (R)-5-(5-(dimethylcarbamoyl)pyridin-2-yl)-7,7-dimethyl-4,5,6,7-tetrahydro-1H-indazole-3-carboxylate

To a solution of rac-ethyl 2-((5R)-5-(5-(dimethylcarbamoyl)pyridin-2-yl)-3,3-dimethyl-2-oxocyclohexyl)-2-oxoacetate (1.86 g, 4.96 mmol) in EtOH (24.8 mL) at room temperature was added K₃PO₄ (944 mg, 6.94 mmol), followed by hydrazine dihydrochloride (407 mg, 5.95 mmol). The reaction mixture was stirred at room temperature for -72 h. The reaction mixture was concentrated in vacuo. The residue was diluted with H₂O and extracted with DCM (1×) then EtOAc (1×). The combined organic layers were washed with saturated NaHCO₃ then concentrated in vacuo to afford caramel oil. This oil was dissolved in DCM then purified by column chromatography (silica gel; 0-15% EtOH: DCM). to provide rac-ethyl (R)-5-(5-(dimethylcarbamoyl)pyridin-2-yl)-7,7-dimethyl-4,5,6,7-tetrahydro-1H-indazole-3-carboxylate (1.33 g, 72.4% yield) as a yellow solid. (LC/MS) m/z (M+H)⁺=371.4. ¹H NMR (600 MHz, CDCl₃) δ (ppm): 8.65 (d, 1H), 7.76 (dd, 1H), 7.32 (d, 1H), 4.34 (q, 2H), 3.35-3.22 (m, 2H), 3.20-2.97 (m, 6H), 2.83 (dd, 1H), 2.04 (t, 1H), 1.95-1.89 (m, 1H), 1.47-1.28 (m, 10H).

### Step 8. Preparation of rac-(R)-5-(5-(dimethylcarbamoyl)pyridin-2-yl)-7,7-dimethyl-4,5,6,7-tetrahydro-1H-indazole-3-carboxylic acid

At 0 °C, to a solution of rac-ethyl (R)-5-(5-(dimethylcarbamoyl)pyridin-2-yl)-7,7-dimethyl-4,5,6,7-tetrahydro-1H-indazole-3-carboxylate (0.510 g, 1.38 mmol) in MeOH (5.51 mL) and THF (5.51 mL) was added a solution of LiOH (116 mg, 2.75 mmol) in H₂O (2.75 mL) dropwise. The reaction mixture was allowed to slowly warm to room temperature then stirred overnight. The mixture was concentrated in vacuo to remove organic solvents, then acidified with HCl to pH=3 which caused precipitation to form. The resulting slurry was extracted with a mixture of chloroform: isopropyl alcohol (3:1, 3 mL×3). The combined organic extracts were concentrated in vacuo and dried further under high vacuum to afford a light-yellow solid. The solid was suspended in toluene then concentrated in vacuo (2×) to give rac-(R)-5-(5-(dimethylcarbamoyl)pyridin-2-yl)-7,7-dimethyl-4,5,6,7-tetrahydro-1H-indazole-3-carboxylic acid (0.443 g, crude) as a solid. The solid was used directly into the next step without further purification. (LC/MS) m/z (M+H)⁺=343.4. ¹H NMR (400 MHz, CD₃OD) δ (ppm): 8.65-8.60 (m, 1H), 7.94 (dd, 1H), 7.64-7.55 (m, 1H), 3.43-3.33 (m, 1H), 3.30-3.20 (m, 1H), 3.13 (s, 3H), 3.06 (s, 3H), 2.88-2.75 (m, 1H), 2.05 (t, 1H), 1.95-1.86 (m, 1H), 1.43 (s, 3H), 1.39 (s, 3H).

### Step 9. Preparation of rac-(R)-5-(5-(dimethylcarbamoyl)pyridin-2-yl)-1-ethyl-7,7-dimethyl-4,5,6,7-tetrahydro-1H-indazole-3-carboxylic acid (P1A)

At 0 °C, to a homogeneous solution of rac-(R)-5-(5-(dimethylcarbamoyl)pyridin-2-yl)-7,7-dimethyl-4,5,6,7-tetrahydro-1H-indazole-3-carboxylic acid (439 mg, 1.28 mmol) in NMP (5.13 mL) was added 1 M LiHMDS in THF (558 mg, 3.33 mL, 3.33 mmol) dropwise. After stirring for 1 h at 0 °C, diethyl sulfate (257 mg, 218 μL, 1.67 mmol) was added. The reaction mixture was warmed to room temperature for stirred overnight. An additional portion of diethyl sulfate (19.8 mg, 16.8 μL, 128 μmol) was added and the reaction mixture was stirred for 10 min at 0 °C, then further stirred at room temperature for 1 h. The reaction mixture was cooled to 0 °C and quenched with H₂O (20 mL), then washed with a mixture of heptane: methyl tert-butyl ether (1:1, 2×20 mL) while cold. The aqueous layer was extracted with DCM (3×30 mL). The combined organic layers were dried over Na₂SO₄, filtered, and concentrated in vacuo to afford a light brown solution, which was dried further under high vacuum to give rac-(R)-5-(5-(dimethylcarbamoyl)pyridin-2-yl)-1-ethyl-7,7-dimethyl-4,5,6,7-tetrahydro-1H-indazole-3-carboxylic acid (0.475 g, crude) as a solution. The solution was used directly in the next step without further purification. (LC/MS) m/z (M+H)⁺=371.4.

### Preparation P2A

### rac-(5R,7R)-5-(5-(Dimethylcarbamoyl)pyridin-2-yl)-1-ethyl-7-methyl-4,5,6,7-tetrahydro-1H-indazole-3-carboxylic acid (P2A)

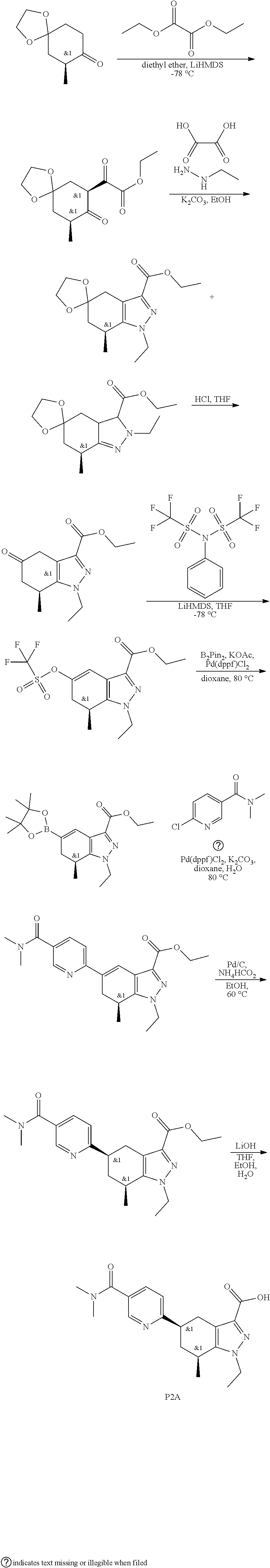

### Step 1. Preparation of rac-ethyl 2-((7R,9S)-9-methyl-8-oxo-1,4-dioxaspiro[4.5]decan-7-yl)-2-oxoacetate

To a round-bottom flask were added rac-(R)-7-methyl-1,4-dioxaspiro[4.5]decan-8-one (CAS: 702-69-2; 5.08 g, 29.8 mmol), diethyl ether (45 mL), and diethyl oxalate (4.36 g, 4.05 mL, 29.8 mmol). The mixture was degassed with nitrogen gas for several min then cooled to −78 °C. At -78 °C, 1 M LiHMDS (5.49 g, 32.8 mL, 32.8 mmol) was added slowly then the mixture was stirred at −78 °C for 2 h. The reaction was quenched with 1 M HCl (80 mL) until the pH adjusted to pH=2 then extracted with EtOAc (2×100 mL). The combined organic layers were dried over Na₂SO₄, filtered, and concentrated in vacuo to afford rac-ethyl 2-((7R,9S)-9-methyl-8-oxo-1,4-dioxaspiro[4.5]decan-7-yl)-2-oxoacetate (7.73 g, crude) as a yellow oil. The oil was used directly in the next step without further purification. (LC/MS) m/z (M+H)⁺=271.0.

### Step 2. Preparation of rac-ethyl (R)-1-ethyl-7-methyl-1,4,6,7-tetrahydrospiro[indazole-5,2′-[1,3]dioxolane]-3-carboxylate and rac-ethyl (R)-2-ethyl-7-methyl-2,4,6,7-tetrahydrospiro[indazole-5,2′-[1,3]dioxolane]-3-carboxylate

To a 250 mL round-bottom flask containing rac-ethyl 2-((7R,9S)-9-methyl-8-oxo-1,4-dioxaspiro[4.5]decan-7-yl)-2-oxoacetate (7.73 g, 28.6 mmol) were added K₂CO₃ (7.91 g, 57.2 mmol) and EtOH (140 mL), followed by ethylhydrazineoxalate (4.29 g, 28.6 mmol). The reaction mixture was stirred at room temperature for 2 h then concentrated in vacuo. To the residue was added H₂O (100 mL) then the mixture was extracted with DCM (2×100 mL). The combined organic layers were washed with brine (50 mL), dried over Na₂SO₄, filtered, and concentrated in vacuo. The crude residue was purified by column chromatography (silica gel; 0-100% EtOAc: heptane) to afford the first eluting isomer: rac-ethyl (R)-2-ethyl-7-methyl-2,4,6,7-tetrahydrospiro[indazole-5,2′-[1,3]dioxolane]-3-carboxylate (1.59 g, 18.9% yield) as a yellow oil and the second eluting isomer: rac-ethyl (R)-1-ethyl-7-methyl-1,4,6,7-tetrahydrospiro[indazole-5,2′-[1,3]dioxolane]-3-carboxylate (4.81 g, 57.1% yield) as a yellow oil. rac-ethyl (R)-1-ethyl-7-methyl-1,4,6,7-tetrahydrospiro[indazole-5,2′-[1,3]dioxolane]-3-carboxylate was brought forward to the next step and rac-ethyl (R)-2-ethyl-7-methyl-2,4,6,7-tetrahydrospiro[indazole-5,2′-[1,3]dioxolane]-3-carboxylate was discarded. rac-ethyl (R)-2-ethyl-7-methyl-2,4,6,7-tetrahydrospiro[indazole-5,2′-[1,3]dioxolane]-3-carboxylate: (LC/MS) m/z (M+H)⁺=295.0. ¹H NMR (400 MHz, (CD₃)₂SO) δ (ppm): 4.53-4.33 (m, 2H), 4.28 (q, 2H), 4.04-3.82 (m, 4H), 2.95-2.51 (m, 3H), 2.04-1.81 (m, 1H), 1.71-1.48 (m, 1H), 1.33-1.22 (m, 9H).

rac-ethyl (R)-1-ethyl-7-methyl-1,4,6,7-tetrahydrospiro[indazole-5,2′-[1,3]dioxolane]-3-carboxylate: (LC/MS) m/z (M+H)⁺=295.4. ¹H NMR (400 MHz, (CD₃)₂SO) δ (ppm): 4.23 (q, 2H), 4.18-3.98 (m, 2H), 3.98-3.83 (m, 4H), 3.20-3.07 (m, 1H), 2.87-2.73 (m, 2H), 2.07-1.96 (m, 1H), 1.77-1.67 (m, 1H), 1.35 (t, 3H), 1.31-1.23 (m, 6H).

### Step 3. Preparation of rac-ethyl (R)-1-ethyl-7-methyl-5-oxo-4,5,6,7-tetrahydro-1H-indazole-3-carboxylate

To a 250 mL round-bottom flask containing rac-ethyl (R)-1-ethyl-7-methyl-1,4,6,7-tetrahydrospiro[indazole-5,2′-[1,3]dioxolane]-3-carboxylate (2.71 g, 9.21 mmol) in THF (18 mL), was added 1 M HCl (1.68 g, 46 mL, 46.0 mmol). The reaction mixture was stirred at room temperature for 19 h then quenched with saturated aqueous NaHCO₃ solution until the pH was adjusted to pH=8. The mixture was extracted with EtOAc (2×75 mL). The combined organic layers were dried over Na₂SO₄, filtered, and concentrated in vacuo. The crude product was purified by column chromatography (silica gel; 0-100% EtOAc: heptane) to afford rac-ethyl (R)-1-ethyl-7-methyl-5-oxo-4,5,6,7-tetrahydro-1H-indazole-3-carboxylate (1.71 g, 74.2% yield) as a yellow oil. (LC/MS) m/z (M+H)⁺=251.4. ¹H NMR (600 MHz, (CD₃)₂SO) δ (ppm): 4.24 (q, 2H), 4.19-4.10 (m, 2H), 3.68-3.56 (m, 1H), 3.56-3.48 (m, 1H), 3.35-3.28 (m, 1H), 2.95 (dd, 1H), 2.35-2.29 (m, 1H), 1.38 (t, 3H), 1.27 (t, 3H), 1.12 (d, 3H).

### Step 4. Preparation of rac-ethyl (R)-1-ethyl-7-methyl-5-(((trifluoromethyl)sulfonyl)oxy)-6,7-dihydro-1H-indazole-3-carboxylate

To a 250 mL round-bottom flask containing rac-ethyl (R)-1-ethyl-7-methyl-5-oxo-4,5,6,7-tetrahydro-1H-indazole-3-carboxylate (1.71 g, 6.83 mmol) in THF (30 mL) was degassed with nitrogen gas and cooled to −78 °C before 1 M LiHMDS (1.26 g, 7.51 mL, 7.51 mmol) was added slowly. The resulting orange solution was stirred for 10 min at −78 °C before a solution of phenyl triflimide (2.68 g, 7.51 mmol) in THF (10 mL) was then added slowly. The reaction mixture was stirred at −78 °C for 15 min before warming to room temperature and stirring for an additional 2 h. The reaction mixture was quenched with H₂O (30 mL) and extracted with EtOAc (2×30 mL). The combined organic layers were dried over Na₂SO₄, filtered, and concentrated in vacuo. The crude product was purified by column chromatography (silica gel; 0-100% EtOAc: heptane) to afford rac-ethyl (R)-1-ethyl-7-methyl-5-(((trifluoromethyl)sulfonyl)oxy)-6,7-dihydro-1H-indazole-3-carboxylate (1.28 g, 49.0% yield) as an orange oil. (LC/MS) m/z (M+H)⁺=383.3. ¹H NMR (400 MHz, (CD₃)₂SO) δ (ppm): 6.78 (d, 1H), 4.33-4.19 (m, 2H), 4.22-4.09 (m, 2H), 3.51-3.39 (m, 1H), 3.27-3.16 (m, 1H), 2.50-2.40 (m, 1H), 1.38 (t, 3H), 1.34-1.26 (m, 3H), 1.25-1.10 (m, 3H).

### Step 5. Preparation of rac-ethyl (R)-1-ethyl-7-methyl-5-(4,4,5,5-tetramethyl-1,3,2-dioxaborolan-2-yl)-6,7-dihydro-1H-indazole-3-carboxylate

A reaction mixture of rac-ethyl (R)-1-ethyl-7-methyl-5-(((trifluoromethyl)sulfonyl)oxy)-6,7-dihydro-1H-indazole-3-carboxylate (1.28 g, 3.35 mmol) were added B₂Pin₂ (1.02 g, 4.02 mmol), KOAc (986 mg, 10.0 mmol), and Pd(dppf)Cl₂ (137 mg, 167 μm) in dioxane (15 mL) was degassed with nitrogen gas for several min then stirred at 80 °C for 2 h before cooling to room temperature. The mixture was filtered through a plug Celite then the filtrate was concentrated in vacuo. The residue was purified by column chromatography (silica gel; 0-100% EtOAc: heptane) to provide rac-ethyl (R)-1-ethyl-7-methyl-5-(4,4,5,5-tetramethyl-1,3,2-dioxaborolan-2-yl)-6,7-dihydro-1H-indazole-3-carboxylate (1.19 g, 98.7% yield) as an orange oil. (LC/MS) m/z (M+H)⁺=361.4.

### Step 6. Preparation of rac-ethyl (R)-5-(5-(dimethylcarbamoyl)pyridin-2-yl)-1-ethyl-7-methyl-6,7-dihydro-1H-indazole-3-carboxylate

A mixture of rac-ethyl (R)-1-ethyl-7-methyl-5-(4,4,5,5-tetramethyl-1,3,2-dioxaborolan-2-yl)-6,7-dihydro-1H-indazole-3-carboxylate (1.19 g, 3.30 mmol) were added 6-chloro-N,N-dimethyl-pyridine-3-carboxamide (915 mg, 4.95 mmol), K₂CO₃ (1.37 g, 9.91 mmol), and Pd(dppf)Cl₂ (135 mg, 165 μmol) in dioxane (13 mL) and H₂O (3.3 mL) was degassed with nitrogen gas for several min. The reaction mixture was heated to 80 °C and stirred for 18 h. After cooling, the mixture was filtered through a plug of Celite. The filtrate was diluted with EtOAc (50 mL) then washed with H₂O (50 mL) and brine (20 mL). The organic layer was dried over Na₂SO₄, filtered, and concentrated in vacuo. The residue was purified by column chromatography (silica gel; 0-10% MeOH: DCM) to afford rac-ethyl (R)-5-(5-(dimethylcarbamoyl)pyridin-2-yl)-1-ethyl-7-methyl-6,7-dihydro-1H-indazole-3-carboxylate (0.748 g, 59.2% yield) as a tan gum. (LC/MS) m/z (M+H)⁺=383.4. ¹H NMR (600 MHz, (CD₃)₂SO) δ (ppm): 8.65-8.60 (m, 1H), 7.90-7.81 (m, 1H), 7.76-7.71 (m, 1H), 7.69 (d, 1H), 4.34-4.26 (m, 2H), 4.25-4.14 (m, 2H), 3.46-3.38 (m, 1H), 3.13 (dd, 1H), 3.02-2.96 (m, 6H), 2.92-2.84 (m, 1H), 1.44-1.38 (m, 3H), 1.37-1.29 (m, 3H), 1.06 (d, 3H).

### Step 7. Preparation of rac-ethyl (5R,7R)-5-(5-(dimethylcarbamoyl)pyridin-2-yl)-1-ethyl-7-methyl-4,5,6,7-tetrahydro-1H-indazole-3-carboxylate

A mixture of rac-ethyl (R)-5-(5-(dimethylcarbamoyl)pyridin-2-yl)-1-ethyl-7-methyl-6,7-dihydro-1H-indazole-3-carboxylate (0.700 g, 1.83 mmol), ammonium formate (462 mg, 7.32 mmol), and Pd/C (195 mg, 183 μmol, 10% wt %) in EtOH (9 mL) was degassed with nitrogen gas for several min then stirred at 60 °C for 1 h. The reaction was cooled to room temperature then filtered through Celite. The filtrate was concentrated in vacuo then purified by column chromatography (silica gel; 0-10% MeOH: DCM) to afford rac-ethyl (5R,7R)-5-(5-(dimethylcarbamoyl)pyridin-2-yl)-1-ethyl-7-methyl-4,5,6,7-tetrahydro-1H-indazole-3-carboxylate (0.587 g, 83.4% yield) as a white foam. (LC/MS) m/z (M+H)⁺=385.4. ¹H NMR (600 MHz, (CD₃)₂SO) δ (ppm): 8.57 (d, 1H), 7.84-7.78 (m, 1H), 7.42 (d, 1H), 4.23-4.08 (m, 3H), 3.17-3.02 (m, 4H), 3.01-2.93 (m, 7H), 2.78-2.68 (m, 1H), 2.26-2.19 (m, 1H), 1.78-1.68 (m, 1H), 1.41-1.35 (m, 2H), 1.30-1.28 (m, 3H), 1.24 (t, 3H).

### Step 8. Preparation of rac-(5R,7R)-5-(5-(dimethylcarbamoyl)pyridin-2-yl)-1-ethyl-7-methyl-4,5,6,7-tetrahydro-1H-indazole-3-carboxylic acid (P2A)

To a reaction mixture of rac-ethyl (5R,7R)-5-(5-(dimethylcarbamoyl)pyridin-2-yl)-1-ethyl-7-methyl-4,5,6,7-tetrahydro-1H-indazole-3-carboxylate (587 mg, 1.53 mmol) in THF (3 mL), EtOH (3 mL), and H₂O (2 mL) was added LiOH (73.1 mg, 3.05 mmol). The reaction mixture was stirred at room temperature for 4.5 h then concentrated in vacuo and acidified to approximately pH=3 using 1 M HCl. The suspension was extracted with a mixture of 10% 2-butanol in DCM (2×20 mL). The combined organic layers were dried over Na₂SO₄, filtered, and concentrated in vacuo to provide rac-(5R,7R)-5-(5-(dimethylcarbamoyl)pyridin-2-yl)-1-ethyl-7-methyl-4,5,6,7-tetrahydro-1H-indazole-3-carboxylic acid (0.547 g, crude) as a yellow foam. The yellow foam was used directly in the next step without further purification. (LC/MS) m/z (M+H)⁺=357.4.

### Preparation P1B

### (S)-5-(Tert-butoxycarbonyl)-7-methyl-4,5,6,7-tetrahydro-1H-pyrazolo[4,3-c]pyridine-3-carboxylic acid

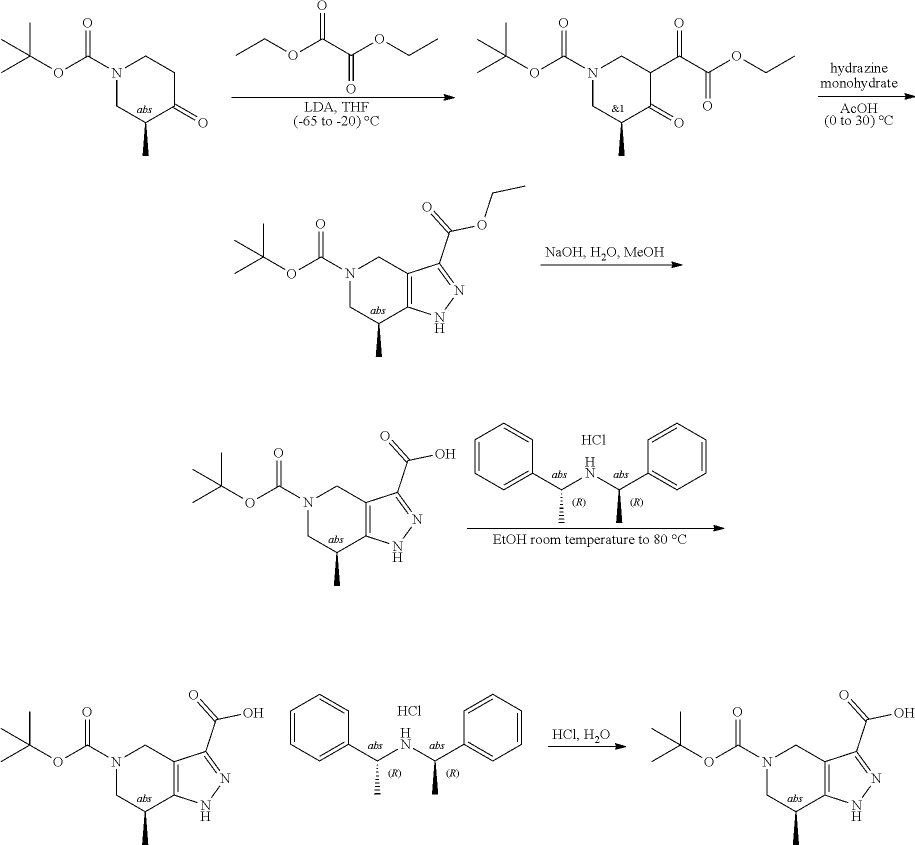

Step 1. Preparation of rac-tert-butyl (5S)-3-(2-ethoxy-2-oxoacetyl)-5-methyl-4-oxopiperidine-1-carboxylate

Under a nitrogen balloon at −65 °C, LDA (44.4 g, 0.414 mol, 207 mL) was added to THF (350 mL) then a solution of tert-butyl (S)-3-methyl-4-oxopiperidine-1-carboxylate (CAS: 2092486-33-2; 88.4 g, 0.414 mol) in THF (350 mL) was added dropwise over 1 hour to keep the inner temperature below −65 °C. The reaction mixture was stirred at −65 °C for 1 hour before a solution of diethyl oxalate (CAS: 95-92-1; 60.6 g, 0.414 mol) in THF (350 mL) was added dropwise at −65 °C. After the addition, the reaction mixture was stirred at −65 °C for 1 hour, then warmed to −20 °C and stirred for 40 min. At 0 °C under nitrogen gas, the reaction mixture was quenched with 1 M aqueous KHSO₄ solution (1415 mL), which caused a white precipitate to form. The suspension was filtered, and then the filtrate was extracted with EtOAc (800 mL×3). The combined organic layer was washed with brine (800 mL), dried over Na₂SO₄, filtered then concentrated in vacuo to provide rac-tert-butyl (5S)-3-(2-ethoxy-2-oxoacetyl)-5-methyl-4-oxopiperidine-1-carboxylate (129 g, 99.2% yield) as a yellow oil. The oil was used directly in the next step without further purification. (LCMS) m/z (M-tert-butyl group)+=257.9. ¹H NMR (400 MHz, (CD₃)₂SO) δ (ppm): 12.21 (br s, 1H), 4.47-4.17 (m, 3H), 4.06-3.47 (m, 2H), 3.29-3.17 (m, 1H), 2.59-2.53 (m, 1H), 1.45-1.37 (m, 9H), 1.28-1.21 (m, 3H), 1.18-0.95 (m, 3H).

### Step 2. Preparation of enriched 5-(tert-butyl) 3-ethyl (S)-7-methyl-1,4,6,7-tetrahydro-5H-pyrazolo[4,3-c]pyridine-3,5-dicarboxylate

At 0 °C, to a suspension of rac-tert-butyl (5S)-3-(2-ethoxy-2-oxoacetyl)-5-methyl-4-oxopiperidine-1-carboxylate (139 g, 0.443 mol) in AcOH (552 mL) was added hydrazine monohydrate (62.7 g, 1.06 mol) which caused the internal temperature to increase to −30 °C. The reaction mixture was stirred at room temperature under nitrogen gas for 1 hour then was diluted with H₂O (500 mL) and saturated NaHCO₃ (1000 mL). The diluted suspension was extracted with EtOAc (800 mL×2). The combined organic layer was dried over Na₂SO₄, filtered then concentrated in vacuo to give a yellow gum. The gum was purified by column chromatography (silica gel, (1:1) EtOAc: petroleum ether) to give enriched 5-(tert-butyl) 3-ethyl (S)-7-methyl-1,4,6,7-tetrahydro-5H-pyrazolo[4,3-c]pyridine-3,5-dicarboxylate (123 g, 89.7% yield) as a yellow gum. (LCMS) m/z (M+H)⁺=310.0. ¹H NMR (400 MHz, (CD₃)₂SO) δ (ppm): 13.71-13.28 (m, 1H), 4.63-4.18 (m, 4H), 3.86-3.59 (m, 1H), 3.12-2.80 (m, 2H), 1.40-1.38 (m, 9H), 1.30-1.24 (m, 3H), 1.17-1.13 (m, 3H). The chiral purity was determined using SFC analytical method on Chiralpak AD-3 100 mm×4.6 mm×3 μm column; Mobile phase A: CO₂/Mobile phase B: EtOH with 0.2% of NH₃; gradient: 5-40% of Mobile Phase B over 3 min then held at 5% of Mobile phase B for 1 min; backpressure: 1500 psi; flow rate: 2.8 mL/min; column temperature: 35 °C to provide enriched 5-(tert-butyl) 3-ethyl (S)-7-methyl-1,4,6,7-tetrahydro-5H-pyrazolo[4,3-c]pyridine-3,5-dicarboxylate: major peak ((SFC-MS) m/z (M-tert-butyl group)+=254.04 at retention time: 2.218 min, 94% ee).

### Step 3. Preparation of enriched (S)-5-(tert-butoxycarbonyl)-7-methyl-4,5,6,7-tetrahydro-1H-pyrazolo[4,3-c]pyridine-3-carboxylic acid

To a solution of enriched 5-(tert-butyl) 3-ethyl (S)-7-methyl-1,4,6,7-tetrahydro-5H-pyrazolo[4,3-c]pyridine-3,5-dicarboxylate (124 g, 0.400 mol) in MeOH (452 mL) and H₂O (452 mL) was added NaOH (32.0 g, 0.800 mol) then stirred at room temperature for 24 hours. The reaction mixture was diluted with H₂O (200 mL) then cooled to 0 °C before the diluted reaction was acidified to pH-3 with 1 M aqueous HCl solution. The acidic reaction mixture was filtered then the filter cake was collected and lyophilized to give enriched (S)-5-(tert-butoxycarbonyl)-7-methyl-4,5,6,7-tetrahydro-1H-pyrazolo[4,3-c]pyridine-3-carboxylic acid (88.6. g, 78.7% yield) as a white solid. The solid was used directly in the next step without further purification. (LCMS) m/z (M+H)⁺=282.0. ¹H NMR (400 MHz, (CD₃)₂SO) δ (ppm): 13.22 (br s, 1H), 4.66-4.34 (m, 2H), 3.86-3.60 (m, 1H), 3.12-2.82 (m, 2H), 1.41 (s, 9H), 1.17 (d, 3H). The chiral purity was determined using SFC analytical method on Chiralpak IG-3 100 mm×4.6 mm×3 μm column; Mobile phase A: CO₂/Mobile phase B: 50% MeOH with 0.2% of NH₃; backpressure: 1500 psi; flow rate: 2.8 mL/min; column temperature: 35 °C to provide enriched (S)-5-(tert-butoxycarbonyl)-7-methyl-4,5,6,7-tetrahydro-1H-pyrazolo[4,3-c]pyridine-3-carboxylic acid: major peak ((SFC-MS) m/z (M-tert-butyl group)+=226.03 at retention time: 0.784 min, 96% ee)

### Step 4. Preparation of (S)-5-(tert-butoxycarbonyl)-7-methyl-4,5,6,7-tetrahydro-1H-pyrazolo[4,3-c]pyridine-3-carboxylic acid (+)-bis[(R)-1-phenylethyl]amine salt

The reaction was conducted in six batches in parallel then combined. To a mixture of enriched (S)-5-(tert-butoxycarbonyl)-7-methyl-4,5,6,7-tetrahydro-1H-pyrazolo[4,3-c]pyridine-3-carboxylic acid (0.180 kg, 0.640 mol) in EtOH (3.4 L) was added (+)-bis[(R)-1-phenylethyl]amine (CAS: 23294-41-9; 144 g, 0.640 mol) in one portion at room temperature to form the first batch. The reaction mixture of the first batch was stirred at room temperature for 10 min then white precipitate formed. The suspension of the first batch was stirred at 80 °C for 1 hour to form a clear solution then stirred at room temperature for 20 hours. The white suspension of the first batch was filtered then the filter cake was washed with EtOH (3×300 mL).

The filter cake from the first batch was collected then combined with the filter cakes from the additional five batches of the same reaction utilizing enriched (S)-5-(tert-butoxycarbonyl)-7-methyl-4,5,6,7-tetrahydro-1H-pyrazolo[4,3-c]pyridine-3-carboxylic acid (0.180 kg, 0.640 mol). The combined filter cake was dried in an oven at 50 °C for 18 hours to provide (S)-5-(tert-butoxycarbonyl)-7-methyl-4,5,6,7-tetrahydro-1H-pyrazolo[4,3-c]pyridine-3-carboxylic acid (+)-bis[(R)-1-phenylethyl]amine salt (1.42 kg, crude) as a white solid. The solid was used directly in the next step without further purification. (LCMS) m/z (M+H)⁺=282.1. ¹H NMR (400 MHz, CD₃OD) δ (ppm): 7.46-7.36 (m, 6H), 7.29-7.23 (m, 4H), 4.76-4.53 (m, 2H), 3.95-3.86 (m, 2H), 3.83-3.70 (m, 1H), 3.25-3.18 (m, 1H), 2.99-2.89 (m, 1H), 1.52 (dd, 6H), 1.48 (s, 9H), 1.25 (d, 3H). The chiral purity was determined using SFC analytical method on Chiralpak IG 50 mm×4.6 mm×3 μm column; Mobile phase A: CO₂/Mobile phase B: MeOH with 0.05% of DEA; gradient: 20-40% of Mobile phase B over 1.5 min and hold 40% of Mobile phase B for 1.0 min then 20% of Mobile Phase B for 0.5 min; flow rate of 4.0 mL/min; backpressure: 1500 psi; column temperature: 35 °C to provide (S)-5-(tert-butoxycarbonyl)-7-methyl-4,5,6,7-tetrahydro-1H-pyrazolo[4,3-c]pyridine-3-carboxylic acid (+)-bis[(R)-1-phenylethyl]amine salt: peak 1 (retention time: 1.019 min, 100% ee).

### Step 5. Preparation of (S)-5-(tert-butoxycarbonyl)-7-methyl-4,5,6,7-tetrahydro-1H-pyrazolo[4,3-c]pyridine-3-carboxylic acid

To a suspension of (S)-5-(tert-butoxycarbonyl)-7-methyl-4,5,6,7-tetrahydro-1H-pyrazolo[4,3-c]pyridine-3-carboxylic acid (+)-bis[(R)-1-phenylethyl]amine salt (48.2 g, 95.1 mmol) in H₂O (482 mL) was added 1 M aqueous HCl solution (99 mL) to pH-1. The reaction mixture was stirred at room temperature for 1 hour then filtered. The filter cake was collected then lyophilized to give (S)-5-(tert-butoxycarbonyl)-7-methyl-4,5,6,7-tetrahydro-1H-pyrazolo[4,3-c]pyridine-3-carboxylic acid (24.3 g, 90.9% yield) as a white solid. The solid was used directly in the next step without further purification. (LCMS) m/z (M+H)⁺=282.0. ¹H NMR (400 MHz, (CD₃)₂SO) δ (ppm): 13.15 (br s, 1H), 4.63-4.47 (m, 1H), 4.44-4.33 (m, 1H), 3.84-3.61 (m, 1H), 3.19-3.00 (m, 1H), 2.94-2.83 (m, 1H), 1.41 (s, 9H), 1.17 (d, 3H). The chiral purity was determined using SFC analytical method on Chiralpak IG-3 100 mm×4.6 mm×3 μm column; Mobile phase A: 50% of CO₂/Mobile phase B: 50% of MeOH with 0.2% NH₃; backpressure: 1500 psi; flow rate: 2.8 mL/min; column temperature: 35 °C to provide (S)-5-(tert-butoxycarbonyl)-7-methyl-4,5,6,7-tetrahydro-1H-pyrazolo[4,3-c]pyridine-3-carboxylic acid: peak 1 ((SFC-MS) m/z (M-tert-butyl group)+=226.00 at retention time: 0.757 min, 99% ee). [α]²⁸D=+11.351 (c=1.5 (g/L in MeOH).

### Preparation P2B

### (S)-5-(5-(Dimethylcarbamoyl)pyridin-2-yl)-1-isopropyl-7-methyl-4,5,6,7-tetrahydro-1H-pyrazolo[4,3-c]pyridine-3-carboxylic acid

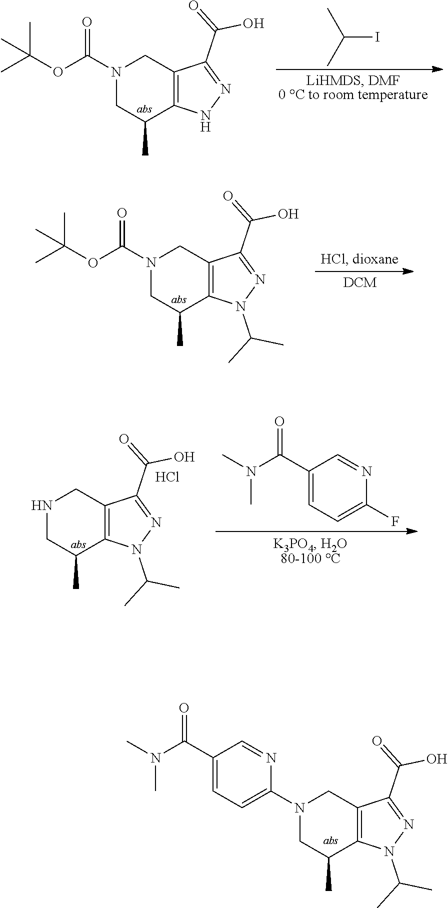

### Step 1. Preparation of (S)-5-(tert-butoxycarbonyl)-1-isopropyl-7-methyl-4,5,6,7-tetrahydro-1H-pyrazolo[4,3-c]pyridine-3-carboxylic acid

At 0 °C under nitrogen gas, to a solution of (S)-5-(tert-butoxycarbonyl)-7-methyl-4,5,6,7-tetrahydro-1H-pyrazolo[4,3-c]pyridine-3-carboxylic acid (12.4 g, 44.1 mmol) in DMF (220 mL) was added 1 M LiHMDS (132 mL). The reaction mixture was warmed to room temperature and stirred for 2 hours before cooling the reaction mixture again to 0 °C. To the cooled reaction mixture was added 2-iodopropane (11.2 g, 66.1 mmol) then the suspension was warmed to room temperature and stirred for 48 hours. The reaction mixture was cooled to 0 °C and another portion of 2-iodopropane (1.12 g, 6.61 mmol) was added. The suspension was warmed to room temperature and stirred for 48 hours. The reaction mixture was cooled to 0 °C then quenched with H₂O (500 mL). The aqueous phase was washed with EtOAc (200 mL×2) then the combined organic layer was discarded. The aqueous layer was acidified with 1 M aqueous HCl to pH-3 then extracted with EtOAc (200 mL×2). The combined organic layer was washed with brine (300 mL×2), dried over Na₂SO₄ and concentrated in vacuo to give (S)-5-(tert-butoxycarbonyl)-1-isopropyl-7-methyl-4,5,6,7-tetrahydro-1H-pyrazolo[4,3-c]pyridine-3-carboxylic acid (13.8 g, 96.8% yield) as a yellow solid. (LCMS) m/z (M+H)⁺=324.0. ¹H NMR (400 MHz, (CD₃)₂SO) δ (ppm): 12.60 (br s, 1H), 4.99-4.73 (m, 1H), 4.57-4.41 (m, 1H), 4.19-3.86 (m, 2H), 3.17-3.00 (m, 2H), 1.42-1.36 (m, 15H), 1.17-1.08 (m, 3H). The chiral purity was determined using SFC analytical method on (S,S) Whelk-01 150 mm×4.6 mm×3.5 μm column; Mobile phase A: CO₂/Mobile phase B: MeOH (0.2% isopropylamine); gradient: 10-50% of Mobile phase B in 6.0 min then held at 10% of Mobile phase B for 2.0 min; backpressure: 2000 psi; flow rate: 1.5 mL/min; column temperature: 35 °C to provide (S)-5-(tert-butoxycarbonyl)-1-isopropyl-7-methyl-4,5,6,7-tetrahydro-1H-pyrazolo[4,3-c]pyridine-3-carboxylic acid: peak 1 (retention time: 2.352 min, 100% ee). [α]³⁴=−100.10 (c=5.4 (g/L) in ACN).

### Step 2. Preparation of (S)-1-isopropyl-7-methyl-4,5,6,7-tetrahydro-1H-pyrazolo[4,3-c]pyridine-3-carboxylic acid hydrochloride

To a solution of (S)-5-(tert-butoxycarbonyl)-1-isopropyl-7-methyl-4,5,6,7-tetrahydro-1H-pyrazolo[4,3-c]pyridine-3-carboxylic acid (12.7 g, 39.3 mmol) in DCM (40 mL) was added 2M HCl in dioxane (500 mL). The reaction mixture was stirred at room temperature for 5 hours then was concentrated in vacuo to provide (S)-1-isopropyl-7-methyl-4,5,6,7-tetrahydro-1H-pyrazolo[4,3-c]pyridine-3-carboxylic acid hydrochloride (9.40 g, crude) as a white solid. The solid was used directly in the next step without further purification. (LCMS) m/z (M+H)⁺=224.2. ¹H NMR (400 MHz, CD₃OD) δ (ppm): 4.68-4.58 (m, 1H), 4.45 (d, 1H), 4.31 (d, 1H), 3.62-3.38 (m, 3H), 1.54 (d, 3H), 1.48 (d, 3H), 1.44 (d, 3H). The chiral purity was determined using SFC analytical method on Chiralcel OX-3 100 mm×4.6 mm×3 μm column; Mobile phase A: CO₂/Mobile phase B: MeOH containing 0.05% of DEA; gradient: 5-40% of Mobile Phase B in 3.0 min then held at 40% of Mobile phase B for 0.9 min then held at 5% of Mobile phase B for 0.1 min; backpressure: 100 bar; flow rate: 2.8 mL/min; column temperature: 40 °C to provide (S)-1-isopropyl-7-methyl-4,5,6,7-tetrahydro-1H-pyrazolo[4,3-c]pyridine-3-carboxylic acid hydrochloride: peak 1 (retention time: 3.037 min, 100% ee). [α]³³=−7.212 (c=1.5 (g/L) in MeOH).

### Step 3. Preparation of (S)-5-(5-(dimethylcarbamoyl)pyridin-2-yl)-1-isopropyl-7-methyl-4,5,6,7-tetrahydro-1H-pyrazolo[4,3-c]pyridine-3-carboxylic acid

To a solution of 6-fluoro-N,N-dimethylnicotinamide (CAS: 1032251-82-3; 2.91 g, 17.3 mmol) and (S)-1-isopropyl-7-methyl-4,5,6,7-tetrahydro-1H-pyrazolo[4,3-c]pyridine-3-carboxylic acid hydrochloride (5.00 g, 19.2 mmol) in H₂O (100 mL) was added K₃PO₄ (12.3 g, 57.7 mmol) at room temperature. The reaction mixture was stirred between 80 to 100 °C for 18 hours then cooled to room temperature. To the suspension was added another portion of 6-fluoro-N,N-dimethylnicotinamide (CAS: 1032251-82-3; 0.227 g, 1.35 mmol) then the reaction mixture was stirred between 80 to 100 °C for 48 hours. The suspension was cooled to room temperature then extracted with EtOAc (100 mL×4). The combined organic layer was discarded. The aqueous layer was acidified to pH=5-6 with aqueous HCl then extracted with (1:10) MeOH:DCM (50 mL×9). The combined organic layer was dried over Na₂SO₄ and concentrated in vacuo to give (S)-5-(5-(dimethylcarbamoyl)pyridin-2-yl)-1-isopropyl-7-methyl-4,5,6,7-tetrahydro-1H-pyrazolo[4,3-c]pyridine-3-carboxylic acid (5.80, crude) as a light-yellow solid. The solid was used directly in the next step without further purification. (LCMS) m/z (M+H)⁺=372.1. ¹H NMR (400 MHz, (CD₃)₂SO) δ (ppm): 12.70 (br s, 1H), 8.26-8.24 (m, 1H), 7.64 (dd, 1H), 6.91 (d, 1H), 5.08 (d, 1H), 4.60-4.49 (m, 1H), 4.43-4.36 (m, 1H), 4.19 (d, 1H), 3.37-3.35 (m, 1H), 3.26-3.21 (m, 1H), 2.98 (s, 6H), 1.40 (dd, 6H), 1.20 (d, 3H). The chiral purity was determined using SFC analytical method on Chiralpak AD-3 100 mm×4.6 mm×3 μm; Mobile phase A: CO₂/Mobile phase B: 40% EtOH with 0.2% of NH₃; backpressure: 1500 psi; flow rate: 2.8 mL/min; column temperature: 35 °C to provide (S)-5-(5-(dimethylcarbamoyl)pyridin-2-yl)-1-isopropyl-7-methyl-4,5,6,7-tetrahydro-1H-pyrazolo[4,3-c]pyridine-3-carboxylic acid: peak 1 ((SFC-MS) m/z (M+H)⁺=372.17 at retention time: 1.254 min, 100% ee).

### Preparation P3B

### (S)-5-(4-(Dimethylcarbamoyl)phenyl)-1-isopropyl-7-methyl-4,5,6,7-tetrahydro-1H-pyrazolo[4,3-c]pyridine-3-carboxylic acid

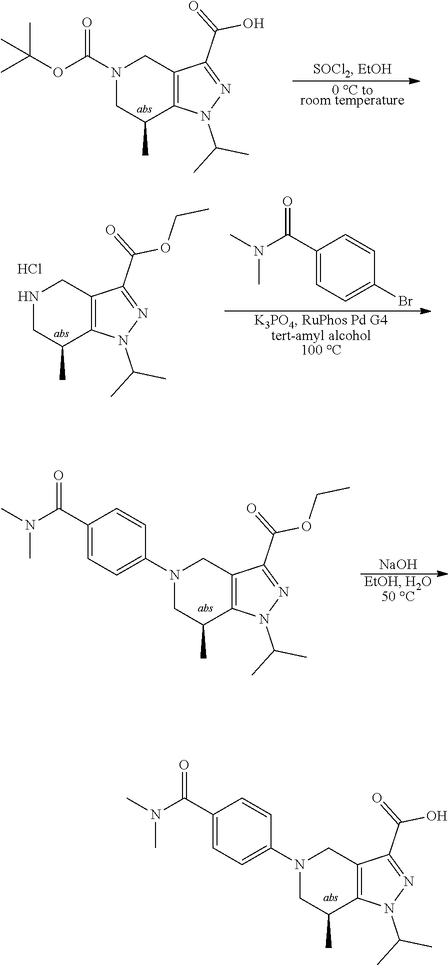

### Step 1. Preparation of ethyl (S)-1-isopropyl-7-methyl-4,5,6,7-tetrahydro-1H-pyrazolo[4,3-c]pyridine-3-carboxylate hydrochloride

To a stirred solution of (S)-5-(tert-butoxycarbonyl)-1-isopropyl-7-methyl-4,5,6,7-tetrahydro-1H-pyrazolo[4,3-c]pyridine-3-carboxylic acid (0.500 g, 1.55 mmol) in EtOH (3 mL) was added SOCl₂ (1.29 g, 10.8 mmol) dropwise at 0 °C. The reaction mixture was stirred at room temperature for 66 hours then concentrated in vacuo to provide ethyl (S)-1-isopropyl-7-methyl-4,5,6,7-tetrahydro-1H-pyrazolo[4,3-c]pyridine-3-carboxylate hydrochloride (0.426 g, 95.7% yield) as a light-yellow solid. (LC/MS) m/z (M+H)⁺=252.2.

### Step 2. Preparation of ethyl (S)-5-(4-(dimethylcarbamoyl)phenyl)-1-isopropyl-7-methyl-4,5,6,7-tetrahydro-1H-pyrazolo[4,3-c]pyridine-3-carboxylate

A reaction mixture of 4-bromo-N,N-dimethylbenzamide (0.344 g, 1.51 mmol), K₃PO₄ (0.667 g, 3.14 mmol) and RuPhos Pd G4 (0.107 g, 0.126 mmol) was degassed with nitrogen gas before a solution of ethyl (S)-1-isopropyl-7-methyl-4,5,6,7-tetrahydro-1H-pyrazolo[4,3-c]pyridine-3-carboxylate hydrochloride (0.362 g, 1.26 mmol) in tert-amyl alcohol (3 mL) was added. The suspension was stirred at 100 °C for 25 hours then filtered and concentrated in vacuo. The residue was dissolved in DCM and purified by column chromatography (silica gel, 20-100% EtOAc: heptane) to provide ethyl (S)-5-(4-(dimethylcarbamoyl)phenyl)-1-isopropyl-7-methyl-4,5,6,7-tetrahydro-1H-pyrazolo[4,3-c]pyridine-3-carboxylate (0.512 g, crude) as a yellow oil. The oil was used directly in the next step without further purification. (LC/MS) m/z (M+H)⁺=399.6. ¹H NMR (600 MHz, (CD₃)₂SO) δ (ppm): 7.38-7.34 (m, 2H), 7.03-6.98 (m, 2H), 4.64-4.56 (m, 1H), 4.53 (d, 1H), 4.31 (q, 2H), 4.11 (d, 1H), 3.88-3.83 (m, 1H), 3.32-3.22 (m, 2H), 2.98 (s, 6H), 1.43 (dd, 6H), 1.34 (t, 3H), 1.31 (d, 3H).

### Step 3. Preparation of (S)-5-(4-(dimethylcarbamoyl)phenyl)-1-isopropyl-7-methyl-4,5,6,7-tetrahydro-1H-pyrazolo[4,3-c]pyridine-3-carboxylic acid

To a solution of ethyl (S)-5-(4-(dimethylcarbamoyl)phenyl)-1-isopropyl-7-methyl-4,5,6,7-tetrahydro-1H-pyrazolo[4,3-c]pyridine-3-carboxylate (0.500 g, 1.25 mmol) in EtOH (3 mL) was added NaOH (0.151 g, 3.76 mmol) and H₂O (1 mL). The reaction mixture was stirred at 50 °C for 25 hours then acidified with HCl and concentrated in vacuo to provide (S)-5-(4-(dimethylcarbamoyl)phenyl)-1-isopropyl-7-methyl-4,5,6,7-tetrahydro-1H-pyrazolo[4,3-c]pyridine-3-carboxylic acid (0.465 g, crude) as a yellow solid. The solid was used directly in the next step without further purification. (LC/MS) m/z (M+H)⁺=371.4. ¹H NMR (600 MHz, (CD₃)₂SO) δ (ppm): 7.37-7.31 (m, 2H), 7.01-6.95 (m, 2H), 4.61-4.52 (m, 1H), 4.50 (d, 1H), 4.11-4.05 (m, 1H), 3.82 (dd, 1H), 3.29 (dd, 1H), 3.24-3.18 (m, 1H), 2.96 (s, 6H), 1.44-1.39 (m, 6H), 1.29 (d, 3H).

### Preparation P4B

### N-(4-(Aminomethyl)phenyl)-4-((6-(2-(2,6-dioxopiperidin-3-yl)-1-oxoisoindolin-4-yl)hex-5-yn-1-yl)oxy)piperidine-1-carboxamide trifluoroacetate

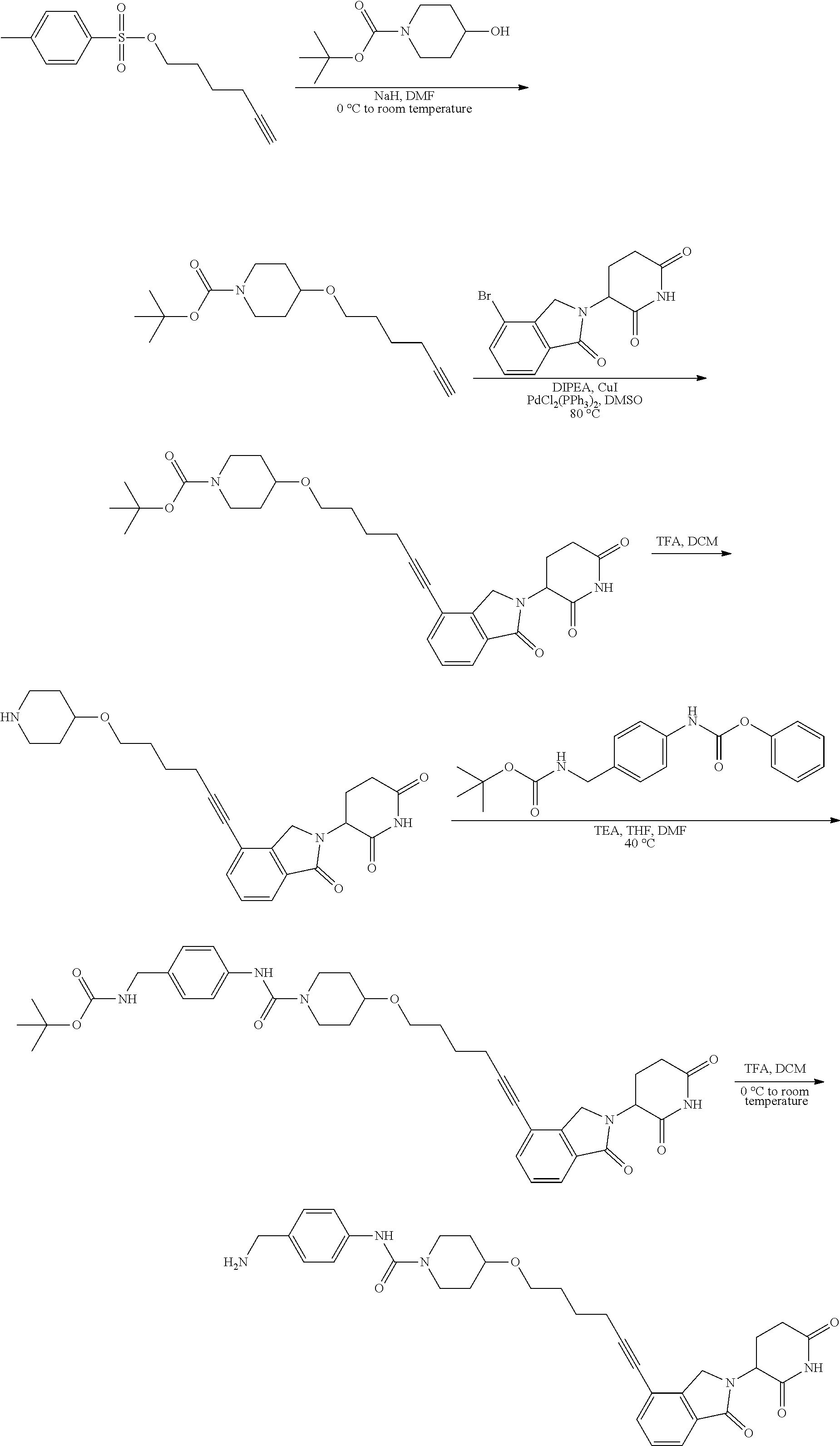

### Step 1. Preparation of tert-butyl 4-(hex-5-yn-1-yloxy)piperidine-1-carboxylate

At 0 °C, to a reaction mixture of tert-butyl 4-hydroxypiperidine-1-carboxylate (CAS: 109384-19-2; 2.00 g, 9.94 mmol) in DMF (30 mL) was added NaH (477 mg, 11.9 mmol). The suspension was stirred at 0 °C for 30 min before hex-5-yn-1-y1 4-methylbenzenesulfonate (CAS: 76911-01-8; 2.51 g, 9.94 mmol) was added. The reaction mixture was warmed to room temperature and stirred for 3 hours. The suspension was cooled to 0 °C then quenched with aqueous NH₄Cl (100 mL) and diluted with EtOAc (200 mL). The organic layer was washed with brine (100 mL), dried with Na₂SO₄, filtered, and concentrated in vacuo to give a yellow oil. The oil was purified by column chromatography (silica gel; 0-5% EtOAc: petroleum ether) to provide tert-butyl 4-(hex-5-yn-1-yloxy)piperidine-1-carboxylate (1.83 g, 65.4% yield) as a colorless oil. (LCMS) m/z (M-(tert-butyl group))+=226.1. ¹H NMR (400 MHz, (CD₃)₂SO) δ (ppm): 3.60 (m, 2H), 3.45-3.37 (m, 3H), 3.08-2.95 (m, 2H), 2.76 (t, 1H), 2.17 (m, 2H), 1.81-1.72 (m, 2H), 1.63-1.44 (m, 4H), 1.39 (s, 9H), 1.36-1.22 (m, 2H).

### Step 2. Preparation of tert-butyl 4-((6-(2-(2,6-dioxopiperidin-3-yl)-1-oxoisoindolin-4-yl)hex-5-yn-1-yl)oxy)piperidine-1-carboxylate

To a reaction mixture of 3-(4-bromo-1-oxoisoindolin-2-yl)piperidine-2,6-dione (CAS: 2093387-36-9; 0.250 g, 0.774 mmol) and tert-butyl 4-(hex-5-yn-1-yloxy)piperidine-1-carboxylate (0.327 g, 1.16 mmol) in DMSO (4 mL) was added DIEA (0.400 g, 3.09 mmol), CuI (29.5 mg, 0.155 mmol) and PdCl₂(PPh₃)₂(0.109 g, 0.155 mmol). The suspension was degassed with nitrogen gas for 3 min then was heated at 80 °C for 3 hours. The reaction mixture was filtered then poured into H₂O (10 mL) and extracted with EtOAc (15 mL×2). The combined organic layer was washed with brine (15 mL×2), dried with Na₂SO₄, filtered, and concentrated in vacuo to give black oil. The oil was purified by column chromatography (silica gel; 0-100% EtOAc: petroleum ether) to provide tert-butyl 4-((6-(2-(2,6-dioxopiperidin-3-yl)-1-oxoisoindolin-4-yl)hex-5-yn-1-yl)oxy)piperidine-1-carboxylate (0.225 g, 55.5% yield) as a black solid. (LCMS) m/z (M-(tert-butyloxycarbonyl protecting group))+=424.1.

### Step 3. Preparation of 3-(1-oxo-4-(6-(piperidin-4-yloxy)hex-1-yn-1-yl)isoindolin-2-yl)piperidine-2,6-dione trifluoroacetate

To a suspension of tert-butyl 4-((6-(2-(2,6-dioxopiperidin-3-yl)-1-oxoisoindolin-4-yl)hex-5-yn-1-yl)oxy)piperidine-1-carboxylate (0.150 g, 0.286 mmol) in DCM (5 mL) was added TFA (3.00 g, 26.3 mmol) at room temperature then stirred for 30 min. The reaction mixture was concentrated in vacuo then lyophilized to provide 3-(1-oxo-4-(6-(piperidin-4-yloxy)hex-1-yn-1-yl)isoindolin-2-yl)piperidine-2,6-dione trifluoroacetate (0.231 g, crude) as a black solid. The solid was used directly in the next step without further purification. (LCMS) m/z (M+H)⁺=424.1.

### Step 4. Preparation of tert-butyl (4-(4-((6-(2-(2,6-dioxopiperidin-3-yl)-1-oxoisoindolin-4-yl)hex-5-yn-1-yl)oxy)piperidine-1-carboxamido)benzyl)carbamate

To a suspension of 3-(1-oxo-4-(6-(piperidin-4-yloxy)hex-1-yn-1-yl)isoindolin-2-yl)piperidine-2,6-dione trifluoroacetate (0.231 g, 0.545 mmol) and phenyl (4-(((tert-butoxycarbonyl)amino)methyl)phenyl)carbamate (CAS: 1632297-04-1; 0.374 g, 1.09 mmol) in THF (6 mL) and DMF (1 mL) was added TEA (0.380 g, 3.75 mmol). The reaction mixture was stirred at 40 °C for 16 hours then poured into H₂O and extracted with EtOAc (10 mL×3). The combined organic phase was washed with brine (10 mL×3) then concentrated in vacuo. The residue was purified by column chromatography (silica gel; 0-100% EtOAc: petroleum ether) to provide tert-butyl (4-(4-((6-(2-(2,6-dioxopiperidin-3-yl)-1-oxoisoindolin-4-yl)hex-5-yn-1-yl)oxy)piperidine-1-carboxamido)benzyl)carbamate (0.232 g, 63.3% yield) as a yellow solid. (LCMS) m/z (M+H)⁺=672.2.

### Step 5. Preparation of N-(4-(aminomethyl)phenyl)-4-((6-(2-(2,6-dioxopiperidin-3-yl)-1-oxoisoindolin-4-yl)hex-5-yn-1-yl)oxy)piperidine-1-carboxamide trifluoroacetate

At 0 °C, to a suspension of tert-butyl (4-(4-((6-(2-(2,6-dioxopiperidin-3-yl)-1-oxoisoindolin-4-yl)hex-5-yn-1-yl)oxy)piperidine-1-carboxamido)benzyl)carbamate (0.253 g, 0.377 mmol) in DCM (5 mL) was added TFA (2.00 g, 17.5 mmol). The reaction mixture was warmed to room temperature then stirred for 30 min. The reaction mixture was concentrated in vacuo then lyophilized to provide N-(4-(aminomethyl)phenyl)-4-((6-(2-(2,6-dioxopiperidin-3-yl)-1-oxoisoindolin-4-yl)hex-5-yn-1-yl)oxy)piperidine-1-carboxamide trifluoroacetate (0.258 g, crude) as a yellow solid. The solid was used directly in the next step without further purification. (LCMS) m/z (M+H)⁺=572.4.

### Preparation P5B

### Phenyl (4-(aminomethyl)-2-fluorophenyl)carbamate hydrochloride

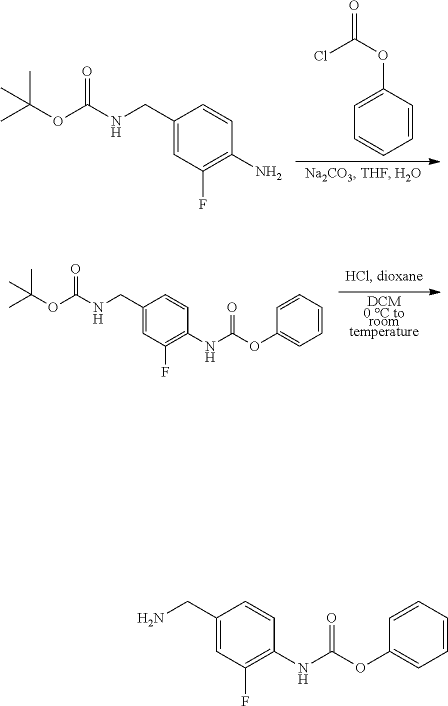

### Step 1. Preparation of phenyl (4-(((tert-butoxycarbonyl)amino)methyl)-2-fluorophenyl)carbamate

To a solution of phenyl chloroformate (CAS: 1885-14-9; 7.53 g, 48.1 mmol) and tert-butyl (4-amino-3-fluorobenzyl)carbamate (CAS: 1823228-30-3; 10.5 g, 43.7 mmol) in THF (50 mL) and H₂O (50 mL) was added Na₂CO₃ (2.78 g, 26.2 mmol). The reaction mixture was stirred at room temperature for 4 hours then filtered. The filtrate was diluted with H₂O (250 mL) and extracted with EtOAc (250 mL×4). The combined organic layer was washed with brine (100 mL×2), dried with Na₂SO₄, filtered and concentrated in vacuo. The residue was triturated with (1:1) EtOAc: petroleum ether (80 mL) at room temperature for 30 min to provide phenyl (4-(((tert-butoxycarbonyl)amino)methyl)-2-fluorophenyl)carbamate (12.0 g, 76.2% yield) as a white solid. (LC/MS) m/z (2M+H)⁺=721.3. ¹H NMR (400 MHz, (CD₃)₂SO) δ (ppm): 9.87 (s, 1H), 7.58 (t, 1H), 7.47-7.38 (m, 2H), 7.32-7.03 (m, 5H), 6.88-6.64 (m, 1H), 4.11 (d, 2H), 1.42-1.37 (m, 9H).

### Step 2. Preparation of phenyl (4-(aminomethyl)-2-fluorophenyl)carbamate hydrochloride

At 0 °C, to a solution of phenyl (4-(((tert-butoxycarbonyl)amino)methyl)-2-fluorophenyl)carbamate (1.50 g, 4.16 mmol) in DCM (10 mL) was added HCl in dioxane (20 mL). The reaction was stirred at room temperature for 16 hours then concentrated in vacuo to provide phenyl (4-(aminomethyl)-2-fluorophenyl)carbamate hydrochloride (1.31 g, crude) as a white solid. ¹H NMR (400 MHz, (CD₃)₂SO) δ (ppm): 10.04 (s, 1H), 8.58 (s, 3H), 7.69 (t, 1H), 7.53-7.38 (m, 3H), 7.34-7.18 (m, 4H), 4.00 (s, 2H).

### Preparation P6B

### 3-(4-(Aminomethyl)-2-fluorophenyl)-1,1-dimethylurea hydrochloride

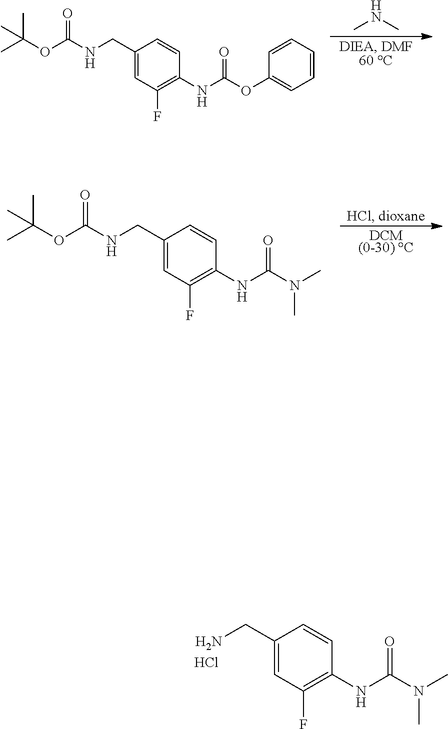

### Step 1. Preparation of tert-butyl (4-(3,3-dimethylureido)-3-fluorobenzyl)carbamate

To a solution of tert-butyl (3-fluoro-4-((phenoxycarbonyl)amino)benzyl)carbamate (0.500 g, 1.39 mmol) and dimethylamine hydrochloride (CAS: 506-59-2; 0.136 g, 1.66 mmol) in DMF (5 mL) was added DIEA (0.538 g, 4.16 mmol). The reaction mixture was stirred at 60 °C for 1 hour then poured into H₂O (10 mL) and filtered. The filter cake was washed with H₂O (2 mL×2) then the filter cake was dissolved with DCM (15 mL). The solution was dried with Na₂SO₄, filtered and concentrated in vacuo to provide tert-butyl (4-(3,3-dimethylureido)-3-fluorobenzyl)carbamate (0.800 g, crude) as a yellow gum. The gum was used directly in the next step without additional purification. (LC/MS) m/z (2M+H)⁺=623.3.

### Step 2. Preparation of 3-(4-(aminomethyl)-2-fluorophenyl)-1,1-dimethylurea hydrochloride

At 0-5 °C, to a stirred suspension of tert-butyl (4-(3,3-dimethylureido)-3-fluorobenzyl)carbamate (0.80 g, 1.4 mmol) in DCM (3 mL) was added 2M HCl in dioxane (15 mL). The reaction mixture was stirred at 30 °C for 1 hour then was concentrated in vacuo. The residue was dissolved in H₂O then extracted with EtOAc (10 mL×2). The aqueous phase was lyophilized to provide 3-(4-(aminomethyl)-2-fluorophenyl)-1,1-dimethylurea hydrochloride (0.32 g, crude) as a white solid. The solid was used directly in the next step without additional purification. (LC/MS) m/z (2M+H)⁺=423.2. ¹H NMR (400 MHz, (CD₃)₂SO) δ (ppm): 8.47 (s, 2H), 8.13 (s, 1H), 7.46 (t, 1H), 7.37 (dd, 1H), 7.21 (dd, 1H), 3.97 (q, 2H), 2.92 (s, 6H).

### Preparation P7B

### 1-isopropyl-7,7-dimethyl-4,5,6,7-tetrahydro-1H-pyrazolo[4,3-c]pyridine-3-carboxylic acid hydrochloride (P7B)

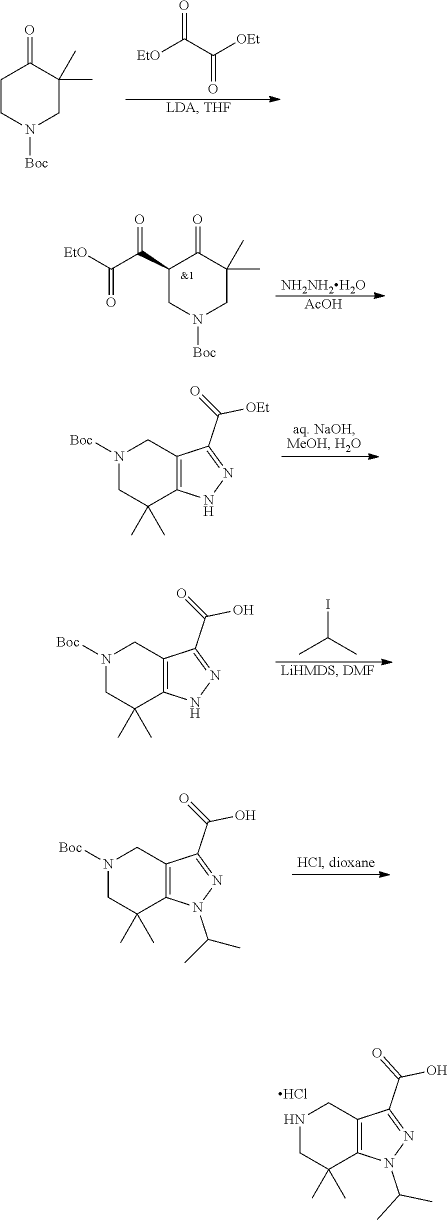

### Step 1: Preparation of rac-tert-butyl (R)-5-(2-ethoxy-2-oxoacetyl)-3,3-dimethyl-4-oxopiperidine-1-carboxylate

Lithium diisopropylamide (7.07 g, 33 mL, 66.0 mmol, 2 M in tetrahydrofuran) was added to tetrahydrofuran (132 mL) under nitrogen atmosphere and the solution was cooled to −65 °C. A solution of tert-butyl 3,3-dimethyl-4-oxopiperidine-1-carboxylate (15.0 g, 66.0 mmol) in tetrahydrofuran (310.5 mL) was then added dropwise while maintaining the temperature at −65 °C, and the mixture was stirred for 60 minutes. Subsequently, a solution of diethyl oxalate (9.64 g, 66.0 mmol) in tetrahydrofuran (82.5 mL) was added dropwise at −65 °C, and the reaction mixture was stirred for an additional 60 minutes. The reaction mixture was then warmed to 25 °C and stirred for 16 hours. The reaction mixture was quenched by the addition of 1 M aqueous potassium bisulfate (450 mL) in an ice bath, and the resulting mixture was filtered to remove the solid. The filtrate was extracted with ethyl acetate (2×200 mL), and the combined organic phases were washed with saturated aqueous sodium chloride solution (500 mL), dried over anhydrous sodium sulfate, filtered, and concentrated under reduced pressure to afford rac-tert-butyl (R)-5-(2-ethoxy-2-oxoacetyl)-3,3-dimethyl-4-oxopiperidine-1-carboxylate (21.6 g, >99% crude) as a yellow oil. (LCMS) m/z (M+H-tert-butyl group)⁺=272.0; ¹H NMR (400 MHz, CDCl₃) δ (ppm): ppm 15.88-15.49 (m, 1H), 4.39 (s, 2H), 4.29 (q, 2H), 3.33 (s, 2H), 1.40 (s, 9H), 1.32 (t, 3H), 1.14 (s, 6H).

### Step 2: Preparation of 5-(tert-butyl) 3-ethyl 7,7-dimethyl-1,4,6,7-tetrahydro-5H-pyrazolo[4,3-c]pyridine-3,5-dicarboxylate

To a mixture of rac-tert-butyl (R)-5-(2-ethoxy-2-oxoacetyl)-3,3-dimethyl-4-oxopiperidine-1-carboxylate (13.6 g, 41.5 mmol) in acetic acid (60 mL) was added hydrazine monohydrate (5.58 g, 95 mmol, 2.3 eq) while maintaining the reaction mixture temperature at approximately 30 °C. using an ice-water bath. The mixture was stirred at 25 °C for 1 hour, then diluted with water (60 mL) and saturated aqueous sodium bicarbonate solution (100 mL). The mixture was extracted with ethyl acetate (3×50 mL), and the combined organic layers were dried over sodium sulfate, filtered, and concentrated under vacuum to afford the crude product (21.3 g) as a yellow gum. This crude product was purified by silica gel chromatography (Eluent: 1:1 petroleum ether/ethyl acetate followed by 100% ethyl acetate) to provide 5-(tert-butyl) 3-ethyl 7,7-dimethyl-1,4,6,7-tetrahydro-5H-pyrazolo[4,3-c]pyridine-3,5-dicarboxylate (12.3 g, 92% yield) as a white solid. (LCMS) m/z (M+H)⁺=324.0; ¹H NMR (400 MHz, (CD₃)₂SO) δ (ppm): ppm 13.48-13.27 (m, 1H) 4.60-4.44 (m, 2H) 4.25 (m, 2H) 3.35 (s, 2H) 1.42 (s, 9H) 1.28 (m, 3H) 1.23-1.17 (m, 6H).

### Step 3: Preparation of 5-(tert-butoxycarbonyl)-7,7-dimethyl-4,5,6,7-tetrahydro-1H-pyrazolo[4,3-c]pyridine-3-carboxylic acid

To a solution of 5-(tert-butyl) 3-ethyl 7,7-dimethyl-1,4,6,7-tetrahydro-5H-pyrazolo[4,3-c]pyridine-3,5-dicarboxylate (12.3 g, 38.0 mmol) in methanol (60 mL) was added a solution of sodium hydroxide (3.04 g, 76.1 mmol) in water (60 mL) at 25 °C. The reaction mixture was stirred at room temperature for 20 h. The solution was then diluted with water (30 mL) and concentrated under reduced pressure to remove methanol. The resulting mixture was acidified to -pH=4 with 1 M HCl, leading to the formation of a solid, allowed to stand for 10 minutes, the mixture was filtered, and the filter cake was collected and concentrated under vacuum to 5-(tert-butoxycarbonyl)-7,7-dimethyl-4,5,6,7-tetrahydro-1H-pyrazolo[4,3-c]pyridine-3-carboxylic acid (9.20 g, 82%) as a white solid. (LCMS) m/z (M+H)⁺=296.0; ¹H NMR (400 MHz, (CD₃)₂SO) δ (ppm): ppm 13.54-12.49 (m, 1H) 4.49 (s, 2H) 3.32 (s, 2H) 1.41 (s, 9H) 1.17 (s, 6H).

### Step 4: Preparation of 5-(tert-butoxycarbonyl)-1-isopropyl-7,7-dimethyl-4,5,6,7-tetrahydro-1H-pyrazolo[4,3-c]pyridine-3-carboxylic acid

To a solution of 5-(tert-butoxycarbonyl)-7,7-dimethyl-4,5,6,7-tetrahydro-1H-pyrazolo[4,3-c]pyridine-3-carboxylic acid (1.0 g, 3.39 mmol) in N,N-dimethylformamide (10 mL) was treated with lithium bis(trimethylsilyl)amide (1.70 g, 10.2 mmol) at 0 °C under a nitrogen atmosphere. The reaction mixture was stirred at 25 °C for 2 hours before 2-iodopropane (1.44 g, 8.46 mmol) was added at 0 °C. The mixture was maintained at 25 °C for 16 hours. Additional 2-iodopropane (230 mg, 1.35 mmol) was added at 0 °C, and the mixture was stirred at 25 °C for another 5 hours. The reaction mixture was quenched with water (80 mL) at 0 °C, and the aqueous phase was washed with ethyl acetate (2×30 mL). The ethyl acetate layer was discarded, and the aqueous phase was acidified with 1 M hydrochloric acid to pH 6, then extracted with ethyl acetate (3×30 mL). The combined organic layers were washed with saturated aqueous sodium chloride solution (3×30 mL), dried over sodium sulfate, and concentrated under reduced pressure to afford a crude yellow solid (1.20 g). The crude product was triturated with a 1:1 mixture of n-hexane and ethyl acetate (6 mL) for 5 hours, filtered, and the filter cake was collected and dried to provide 5-(tert-butoxycarbonyl)-1-isopropyl-7,7-dimethyl-4,5,6,7-tetrahydro-1H-pyrazolo[4,3-c]pyridine-3-carboxylic acid (800 mg, 70% yield) as a white solid. (LCMS) m/z (M+H)⁺=338.1; ¹H NMR (400 MHz, (CD₃)₂SO) δ (ppm): ppm 12.66 (br s, 1H) 4.81-4.58 (m, 1H) 4.49-4.32 (m, 2H) 3.33 (s, 2H) 1.41 (s, 9H) 1.38 (s, 6H) 1.28 (s, 6H).

### Step 5: Preparation of 1-isopropyl-7,7-dimethyl-4,5,6,7-tetrahydro-1H-pyrazolo[4,3-c]pyridine-3-carboxylic acid hydrochloride

To a solution of 5-(tert-butoxycarbonyl)-1-isopropyl-7,7-dimethyl-4,5,6,7-tetrahydro-1H-pyrazolo[4,3-c]pyridine-3-carboxylic acid (0.8 g, 2.37 mmol) in dichloromethane (5 mL) was added ~0.1 M hydrochloric acid in 1,4-dioxane (20 mL) at 0 °C. The reaction mixture was stirred at 20-30 °C. for 2 h, the residue was dissolved in water and then lyophilized to provide 1-isopropyl-7,7-dimethyl-4,5,6,7-tetrahydro-1H-pyrazolo[4,3-c]pyridine-3-carboxylic acid hydrochloride (540 mg, 83% yield) as a white solid; (LCMS) m/z (M+H)⁺=238.0; ¹H NMR (400 MHz, (CD₃)₂SO) δ (ppm): ppm 13.23-12.73 (m, 1H), 9.77-9.36 (m, 2H), 4.83-4.67 (m, 1H), 4.16 (s, 2H), 3.18 (s, 2H), 1.47-1.43 (m, 6H), 1.43-1.40 (m, 6H).

### Preparation P8B

### rac-(R)—N-(4-(Aminomethyl)-2-fluorophenyl)-4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamide bis(2,2,2-trifluoroacetate) (P8B)

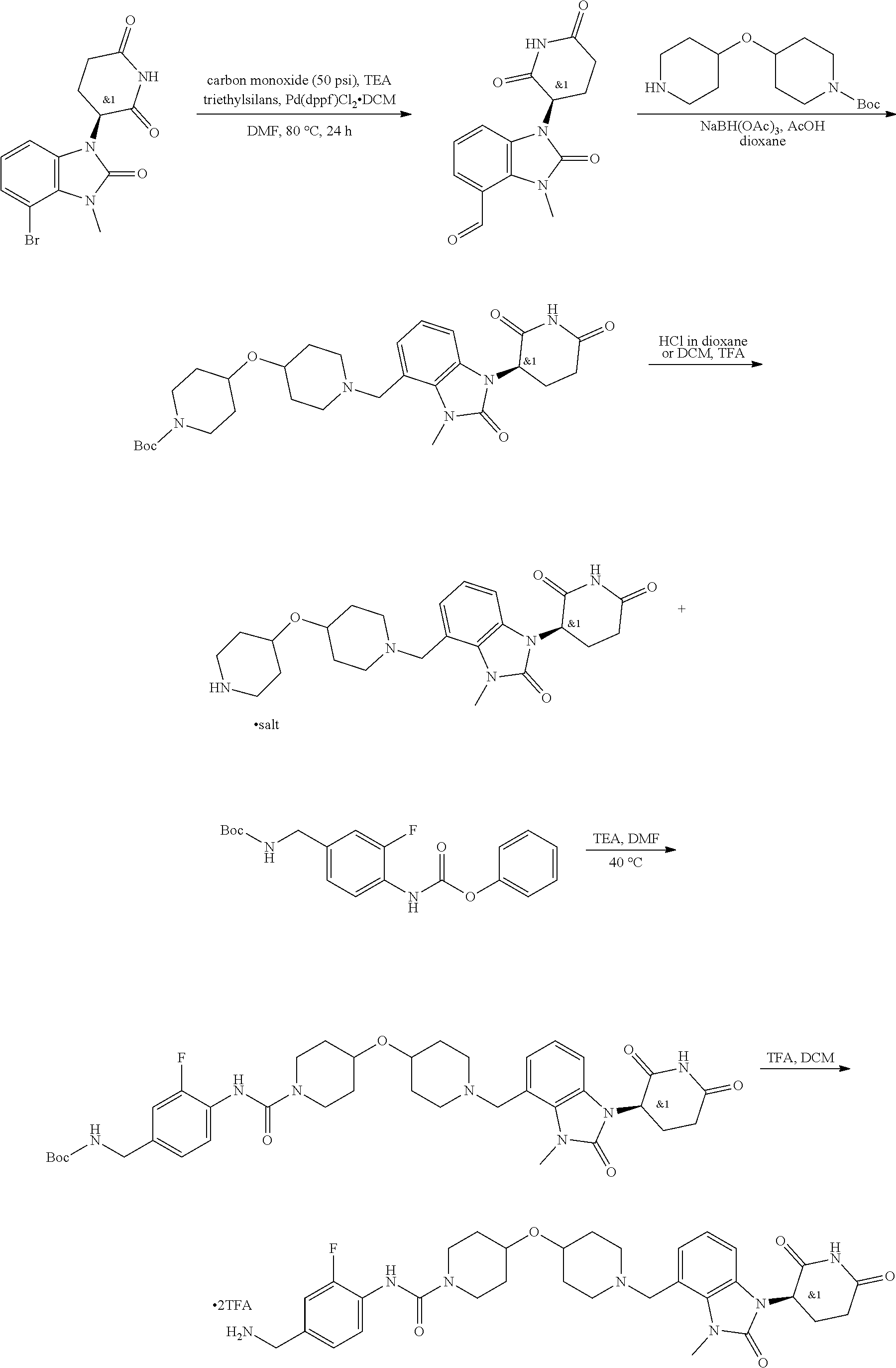

### Step 1: Preparation of rac-(R)-1-(2,6-dioxopiperidin-3-yl)-3-methyl-2-oxo-2,3-dihydro-1H-benzo[d]imidazole-4-carbaldehyde

The reaction was carried out in three parallel batches and then combined for purification. To a solution of rac-(R)-3-(4-bromo-3-methyl-2-oxo-2,3-dihydro-1H-benzo[d]imidazol-1-yl)piperidine-2,6-dione (CAS: 2304754-51-4, 50 g, 147.86 mmol) in DMF (750 mL) was added TEA (44.89 g, 443.58 mmol, 61.74 mL), Pd(dppf)Cl₂ (10.82 g, 14.79 mmol), and triethylsilane (51.58 g, 443.58 mmol, 70.85 mL) at room temperature. After addition, the reaction was degassed under vacuum and purged with argon gas (3 times), then stirred under carbon monoxide gas at 50 psi at 80 °C for 24 h.

The three reaction batches were combined and cooled between 0-10 °C before water (2.5 L) was added dropwise, which caused solid to precipitate out of solution. The suspension was filtered, and washed with water (3×500 mL). The filter cake was triturated with ACN (500 mL) at 95 °C for 1 h, and then stirred at 25 °C for 12 h, filtered. The filter cake was re-triturated with ACN (500 mL) at 95 °C for 1 h, and then stirred at 25 °C for 12 h, filtered and the filter cake was dried under vacuum to give rac-(R)-1-(2,6-dioxopiperidin-3-yl)-3-methyl-2-oxo-2,3-dihydro-1H-benzo[d]imidazole-4-carbaldehyde (51 g, 40% yield) as a gray solid. (LCMS) m/z (M+H)⁺=288.1; ¹H NMR (400 MHz, (CD₃)₂SO) δ (ppm): ppm 11.15 (br s, 1H), 10.40 (s, 1H), 7.60 (m, 1H), 7.46 (m, 1H), 7.21 (m, 1H), 5.47 (m, 1H), 3.67 (s, 3H), 2.97-2.84 (m, 1H), 2.81-2.71 (m, 1H), 2.65 (m, 1H), 2.13-2.03 (m, 1H).

### Step 2: Preparation of rac-tert-butyl (R)-4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxylate

rac-(R)-1-(2,6-dioxopiperidin-3-yl)-3-methyl-2-oxo-2,3-dihydro-1H-benzo[d]imidazole-4-carbaldehyde (12.0 g, 70% wt, 29.2 mmol) was added to a stirred solution of sodium triacetoxyborohydride (9.58 g, 97% wt, 43.9 mmol) and 4-[(piperidin-4-yl)oxy]piperidine-1-carboxylic acid tert-butyl ester (11.4 g, 95% wt, 38.0 mmol) in 1,4-dioxane (200 mL) at room temperature. After 15 minutes, acetic acid (2.63 g, 2.51 mL, 43.9 mmol) was added, and the reaction mixture was stirred for 30 minutes. A second portion of sodium triacetoxyborohydride (9.58 g, 97% wt, 43.9 mmol) was added, and the reaction mixture was stirred for 18 h. Additional 4-[(piperidin-4-yl)oxy]piperidine-1-carboxylic acid tert-butyl ester (1.66 g, 5.85 mmol) and sodium triacetoxyborohydride (1.05 g, 4.97 mmol) were added, and the reaction mixture was stirred for 5 h. Further sodium triacetoxyborohydride (6.51 g, 30.7 mmol) was added, and the reaction mixture was stirred for another 18 h. Water (12.7 mL) was added slowly (exotherm), followed by methyl tert-butyl ether (200 mL). The reaction mixture was filtered through diatomaceous earth, and the filtrate was concentrated. The resulting residue was treated with dichloromethane (150 mL), silica gel (15 g), and heptane (150 mL), stirred, and filtered. The filtrate was concentrated, ethanol (60 mL) was added, and the solution was concentrated again. The residue was recrystallized using ethanol (180 mL) at 70 °C for 1.5 h and then stirred at room temperature for 18 h. The solid was filtered, washed with ethanol, ethyl acetate, and heptane, and dried to provide first batch of the title compound as a gray solid (11.8 g, 72% yield), ethanol filtrate provided additional second batch (580 mg, 3.6% yield) as a white solid. The batches were combined to provide rac-tert-butyl (R)-4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxylate (12.38 g, 76% yield). The solid was dissolved in mixture of DCM (120 ml), 1,4-dioxane (30 mL) and treated with silicycle thiol (2.5 g). The reaction mixture was stirred at reflux (52 °C. external) for 3.5 h, then slowly cooled to room temperature and stirred for an additional 18 h. Silica gel (6 g) was added, reaction stirred at room temperature for 20 minutes and then filtered through a small pad of silica gel, solvent concentrated in vacuum to provide a viscous liquid used directly in the next step. (LCMS) m/z (M+H)⁺=556.3.

### Step 3: Preparation of rac-(R)-3-(3-methyl-2-oxo-4-((4-(piperidin-4-yloxy)piperidin-1-yl)methyl)-2,3-dihydro-1H-benzo[d]imidazol-1-yl)piperidine-2,6-dione dihydrochloride

A mixture of rac-tert-butyl (R)-4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxylate (12.2 g, 21.95 mmol) was dissolved in dioxane (100 mL). The reaction mixture was stirred at an internal temperature of 50 °C, and hydrochloric acid (4 M in dioxane, 4.0 g, 27.4 mL, 109.78 mmol) was added in one portion at 50 °C, resulting in immediate dissolution of the solid. After approximately 1 minute, a new precipitate formed rapidly, and the reaction mixture was stirred at 50 °C for 22 h. The solid was filtered off, washed with dioxane and then heptane, and dried on the filter under vacuum suction for 10 minutes, followed by drying in a vacuum oven at 45 °C for 3 h to afford rac-(R)-3-(3-methyl-2-oxo-4-((4-(piperidin-4-yloxy)piperidin-1-yl)methyl)-2,3-dihydro-1H-benzo[d]imidazol-1-yl)piperidine-2,6-dione dihydrochloride (12.9 g, 74.4% purity, 82.8% yield) as a white solid. (LCMS) m/z (M+H)⁺=456.3.

### Step 4: Preparation of rac-tert-butyl (R)-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)carbamate

To a solution of rac-(R)-3-(3-methyl-2-oxo-4-((4-(piperidin-4-yloxy)piperidin-1-yl)methyl)-2,3-dihydro-1H-benzo[d]imidazol-1-yl)piperidine-2,6-dione bis(2,2,2-trifluoroacetate) (5.0 g, 7.32 mmol) and phenyl (4-(((tert-butoxycarbonyl)amino)methyl)-2-fluorophenyl)carbamate (2.64 g, 7.31 mmol, P5, step-1) in N,N-dimethylformamide (30.0 mL) was added triethylamine (2.96 g, 3.99 mL, 29.3 mmol). The reaction mixture was heated at 40 °C for 2 h. The resulting mixture was poured into water and filtered, and the solid was lyophilized to provide title compound (4.80 g, 91% yield) as a white solid (LCMS) m/z (M+H)⁺=722.2; ¹H NMR (400 MHz, (CD₃)₂SO) δ (ppm): =11.11 (s, 1H), 8.25-8.18 (m, 1H), 7.44-7.38 (m, 1H), 7.33-7.27 (m, 1H), 7.09-7.05 (m, 1H), 7.02-6.94 (m, 3H), 6.91-6.86 (m, 1H), 5.42-5.33 (m, 1H), 4.10-4.06 (m, 2H), 3.80-3.74 (m, 2H), 3.71-3.67 (m, 3H), 3.65-3.58 (m, 3H), 3.53-3.42 (m, 2H), 3.12-3.03 (m, 2H), 2.95-2.88 (m, 1H), 2.78-2.73 (m, 1H), 2.72-2.60 (m, 4H), 2.57-2.53 (m, 2H), 2.20-2.09 (m, 2H), 2.07-1.95 (m, 1H), 1.84-1.71 (m, 4H), 1.40 (s, 9H).

### Step 5: Preparation of rac-(R)—N-(4-(aminomethyl)-2-fluorophenyl)-4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamide bis(2,2,2-trifluoroacetate)

To a solution of rac-tert-butyl (R)-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)carbamate (1 g, 1.385 mmol) in dichloromethane (9 mL) was added trifluoroacetic acid (3 mL) at 25 °C, and the reaction mixture was stirred at 25 °C for additional 16 h. The reaction solution was concentrated under reduced pressure, and the residue was dried under high vacuum for 3 h to afford the title compound (1.45 g, yield >99%) as a white solid which was used directly in the next step. (LCMS) m/z (M+H)⁺=622.3. ¹H NMR (400 MHz, (CD₃)₂SO) δ (ppm): ppm 11.14 (s, 1H) 9.27-9.53 (m, 1H) 8.39 (m, 1H) 8.19 (br s, 4H) 7.42-7.48 (m, 1H) 7.27-7.33 (m, 2H), 7.18 (m, 3H) 5.45 (m, 1H) 4.64 (m, 2H) 4.01 (m, 3H) 3.76-3.91 (m, 3H) 3.64-3.73 (m, 2H) 3.26-3.40 (m, 2H) 3.07-3.23 (m, 4H) 2.07-2.18 (m, 1H) 1.98-2.06 (m, 1H), 1.91-1.97 (m, 1H) 1.83 (br s, 4H) 1.53-1.67 (m, 1H) 1.30-1.51 (m, 3H) 1.18 (t, 1H).

### Example 1

### N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide (1)

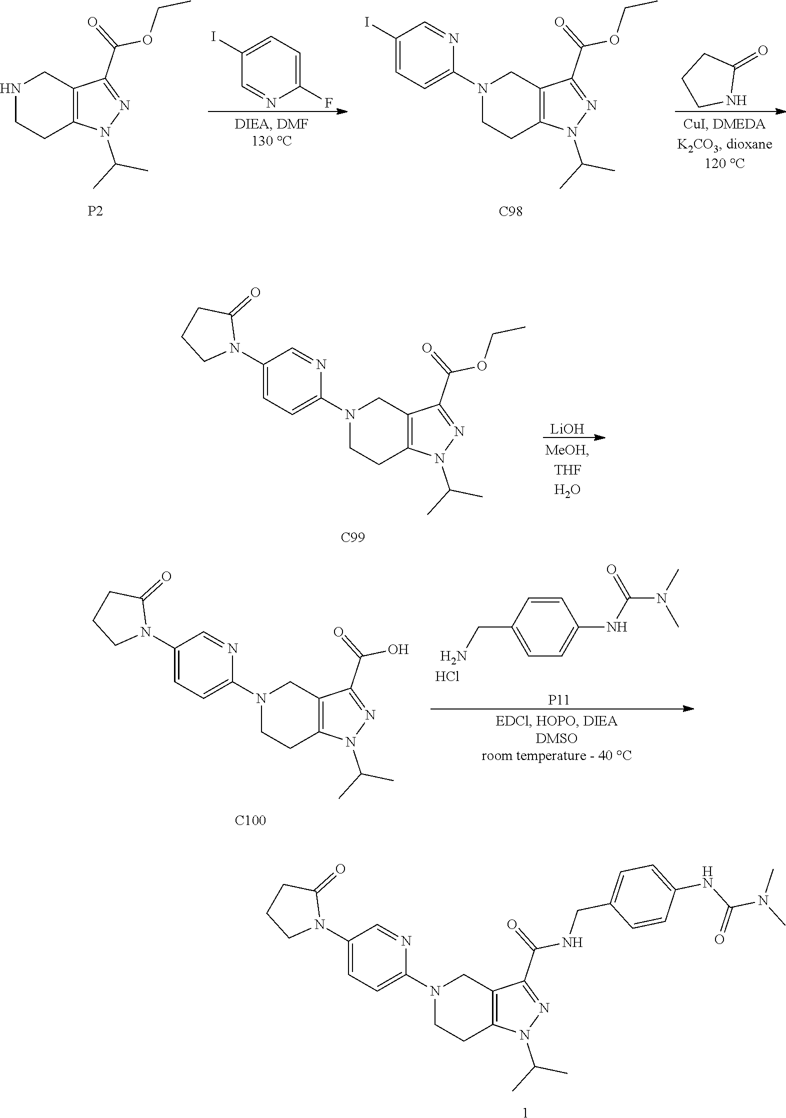

### Step 1. Preparation of ethyl 5-(5-iodopyridin-2-yl)-1-isopropyl-4,5,6,7-tetrahydro-1H-pyrazolo[4,3-c]pyridine-3-carboxylate (C98)

A mixture of P2 (1.36 g, 4.98 mmol), 2-fluoro-5-iodopyridine (CAS: 171197-80-1, 1.22 g, 5.48 mmol), and DIEA (1.93 g, 14.9 mmol) in DMF (25.0 mL) was stirred at 130 °C for 16 h. The brown reaction mixture was concentrated in vacuo and purified by column chromatography (silica gel, 0-20% EtOAc: PE) to provide C98 (0.61 g, 27.8%) as a white solid. (LC/MS) m/z (M+H)⁺=441.2. ¹H NMR (400 MHz, CDCl₃) δ (ppm): 8.32-8.31 (m, 1H), 7.68 (dd, 1H), 6.58 (d, 1H), 4.60 (s, 2H), 4.52-4.38 (m, 3H), 4.03 (t, 2H), 2.80 (t, 2H), 1.52 (d, 6H), 1.44-1.40 (m, 3H).

### Step 2. Preparation of ethyl 1-isopropyl-5-(5-(2-oxopyrrolidin-1-yl)pyridin-2-yl)-4,5,6,7-tetrahydro-1H-pyrazolo[4,3-c]pyridine-3-carboxylate (C99)

A mixture of C98 (0.400 g, 0.909 mmol), pyrrolidine-2-one (0.116 g, 1.36 mmol), CuI (34.6 mg, 0.182 mmol), DMEDA (16.0 mg, 0.182 mmol) and K₂CO₃ (0.377 g, 2.73 mmol) in dioxane (10.0 mL) was heated at 120 °C for 16 h under nitrogen. The reaction was concentrated in vacuo, and then the residue was purified by column chromatography (silica gel, 0-30% THF:PE) to provide C99 (0.36 g, 99.7% yield) as a white solid. (LC/MS) m/z (M+H)⁺=398.2.

### Step 3. Preparation of 1-isopropyl-5-(5-(2-oxopyrrolidin-1-yl)pyridin-2-yl)-4,5,6,7-tetrahydro-1H-pyrazolo[4,3-c]pyridine-3-carboxylic acid (C100)

To a solution of C99 (0.360 g, 0.906 mmol) in THF (5.00 mL), MeOH (1.25 mL) and H₂O (2.50 mL) was added LiOH (57.5 mg, 2.40 mmol) then stirred at room temperature for 1 h and 30 min. The solution was concentrated in vacuo and then acidified to pH=5-6 with 2N HCl. An off-white solid precipitated was filtered, then washed with H₂O (3×50 mL), dried, then lyophilized to provide C100 (0.27 g, 80.7%) as a white solid. ¹H NMR (400 MHz, CD₃OD) δ (ppm): 8.29 (d, 1H), 7.82 (dd, 1H), 6.91 (d, 1H), 4.67 (s, 2H), 4.60-6.45 (m, 1H), 3.96 (t, 2H), 3.88-3.83 (m, 2H), 2.86 (t, 2H), 2.60-2.52 (m, 2H), 2.24-2.13 (m, 2H), 1.47 (d, 6H).

### Step 4. Preparation of N-(4-(3,3-dimethylureido)benzyl)-1-isopropyl-5-(5-(2-oxopyrrolidin-1-yl)pyridin-2-yl)-4,5,6,7-tetrahydro-1H-pyrazolo[4,3-c]pyridine-3-carboxamide (1)

A solution of C100 (80.0 mg, 0.217 mmol), HOPO (71.0 mg, 0.639 mmol), EDCI (99.6 mg, 0.520 mmol) and DIEA (0.140 g, 1.08 mmol) in DMSO (2.0 mL) were stirred at room temperature for 10 min. to the reaction mixture was added P11 (74.6 mg, 0.325 mmol) then heated to 40 °C and stirred for 2 h. The residue was purified by reverse phase HPLC (C₁₈ 150 mm×30 mm×5 μm column, Mobile Phase A: H₂O/Mobile Phase B: ACN (NH₄OH—NH₄HCO₃), 18 to 58% of Mobile Phase B over 9 min, 100% of Mobile Phase B hold 2 min, flow rate: 30 mL/min) and lyophilized to provide 1 (72.2 mg, 61.2% yield) as a white solid. (LC/MS) m/z (M+H)⁺=545.4. ¹H NMR (400 MHz, (CD₃)₂SO) δ (ppm): 8.33-8.27 (m, 2H), 8.23 (s, 1H), 7.89 (dd, 1H), 7.42-7.37 (m, 2H), 7.20-7.15 (m, 2H), 6.90 (d, 1H), 4.65 (s, 2H), 4.53-4.42 (m, 1H), 4.35 (d, 2H), 3.87 (t, 2H), 3.76 (t, 2H), 2.91 (s, 6H), 2.80 (t, 2H), 2.43 (t, 2H), 2.10-2.00 (m, 2H), 1.39 (d, 6H).

### Example 2

### 5-(5-(Dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide (2)

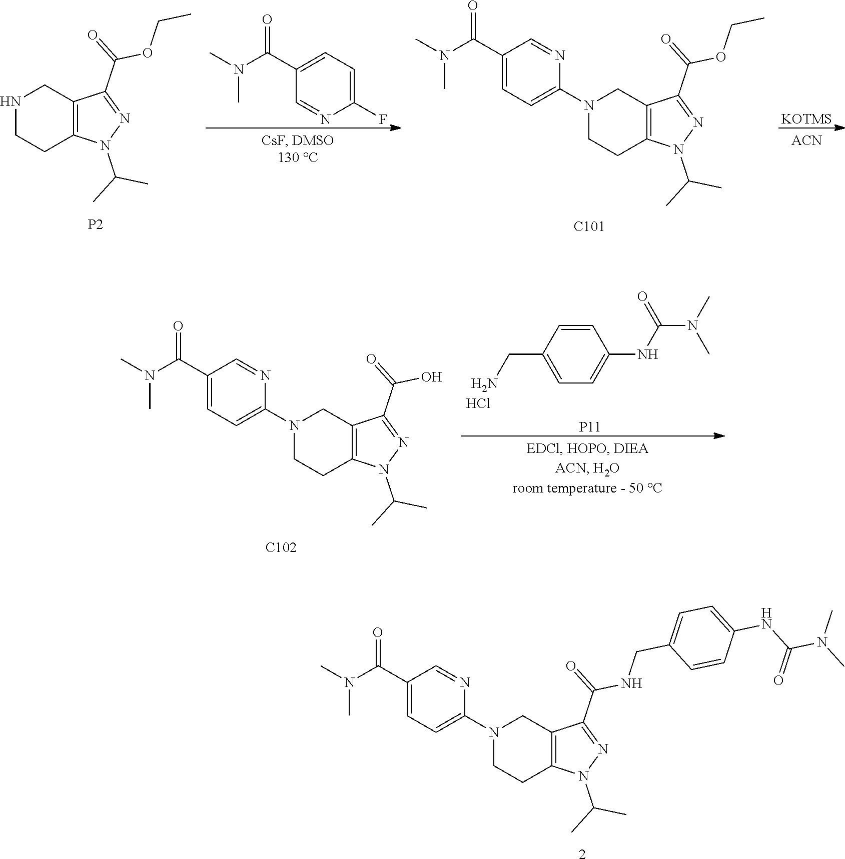

### Step 1. Preparation of ethyl 5-(5-(dimethylcarbamoyl)pyridin-2-yl)-1-isopropyl-4,5,6,7-tetrahydro-1H-pyrazolo[4,3-c]pyridine-3-carboxylate (C101)

To a mixture of P2 (70.0 g, 256 mmol) and CsF (97.1 g, 639 mmol) in DMSO (0.7 L) was added 6-fluoro-N,N-dimethylnicotinamide (CAS: 1032251-82-3, 57.3 g, 256 mmol). The reaction was stirred at 130 °C for 8 h. The reaction was diluted with H₂O (6 L) and then extracted with MeOH: DCM (1:10, 3×2 L). The combined organic layer was washed with brine (2×3 L), dried with Na₂SO₄, filtered, and concentrated in vacuo. The residue was purified by column chromatography (silica gel, 0-100% EtOAc: PE) to provide C101 (72.0 g, 73.1% yield) as a pale yellow solid. (LC/MS) m/z (M+H)⁺=386.2. ¹H NMR (600 MHz, (CD₃)₂SO) δ (ppm): 8.25 (d, 1H), 7.65 (dd, 1H), 6.91 (d, 1H), 4.74 (s, 2H), 4.58-4.47 (m, 1H), 4.30 (q, 2H), 3.96 (t, 2H), 2.97 (s, 6H), 2.83 (t, 2H), 1.38 (d, 6H), 1.32 (t, 3H).

### Step 2. Preparation of 5-(5-(dimethylcarbamoyl)pyridin-2-yl)-1-isopropyl-4,5,6,7-tetrahydro-1H-pyrazolo[4,3-c]pyridine-3-carboxylic acid (C102)

The reaction was conducted in two batches then combined for purification. At room temperature under nitrogen, a mixture of C101 (42.0 g, 109 mmol) in ACN (420 mL) was added KOTMS (28.0 g, 218 mmol) then stirred at room temperature for 1 h to afford the first batch. The first batch was acidified with 2M HCl in dioxane (109 mL, 7.95 g, 218 mmol) to pH=5-6 then concentrated in vacuo.

The first batch's residue was combined with the second batch of the same reaction utilizing C101 (43.0 g, 112 mmol). The combined reaction mixture was diluted with brine (500 mL) and extracted with MeOH:DCM (1:10, 3×1 L). The combined organic layers were dried with Na₂SO₄, filtered, and then concentrated in vacuo to provide C102 (66.0 g, 83.7% yield) as a white solid. This was used in the next step without further purification. ¹H NMR (600 MHz, (CD₃)₂SO) δ (ppm): 8.24 (d, 1H), 7.63 (dd, 1H), 6.92 (d, 1H), 4.70 (s, 2H), 4.54-4.37 (m, 1H), 3.94 (t, 2H), 2.97 (s, 6H), 2.79 (t, 2H), 1.37 (t, 6H).

### Step 3. Preparation of 5-(5-(dimethylcarbamoyl)pyridin-2-yl)-N-(4-(3,3-dimethylureido)benzyl)-1-isopropyl-4,5,6,7-tetrahydro-1H-pyrazolo[4,3-c]pyridine-3-carboxamide (2)

To C102 (70 mg, 0.20 mmol) dissolved in ACN (1.0 mL) and H₂O (0.1 mL) was added HOPO (26 mg, 0.24 mmol) and EDCI (60 mg, 0.31 mmol). The reaction was stirred for 20 min at 50 °C and then cooled to room temperature before DIEA (76 mg, 0.59 mmol) and P11 (54 mg, 0.24 mmol) were added to the reaction. The mixture was stirred at room temperature for 1 h and 20 min. The reaction was concentrated in vacuo, dissolved in DMSO, and acidified by TFA. The acidic residue was purified by reverse phase HPLC (Sunfire C₁₈ 100 mm×19 mm×5 μm column, Mobile Phase A: H₂O/Mobile Phase B: ACN (0.05% TFA), 15 to 95% of Mobile Phase B over 9.0 min, 95% of Mobile Phase B hold 1.0 min, flow rate: 25 mL/min) to provide 2 (30 mg, 25% yield). (LC/MS) m/z (M+H)⁺=533.4. ¹H NMR (600 MHz, (CD₃)₂SO) δ (ppm): =8.27 (t, 1H), 8.18-8.12 (m, 2H), 7.58 (dd, 1H), 7.34-7.30 (m, 2H), 7.11 (d, 2H), 6.84 (d, 1H), 4.68 (s, 2H), 4.45-4.38 (m, 1H), 4.28 (d, 2H), 3.89 (t, 2H), 2.93-2.88 (m, 6H), 2.84 (s, 6H), 2.76 (t, 2H), 1.33 (d, 6H).

### Example 3

### 5-(5-(Dimethylcarbamoyl)pyridin-2-yl)-N-(4-(3,3-dimethylureido)benzyl)-1-ethyl-4,5,6,7-tetrahydro-1H-pyrazolo[4,3-c]pyridine-3-carboxamide (3)

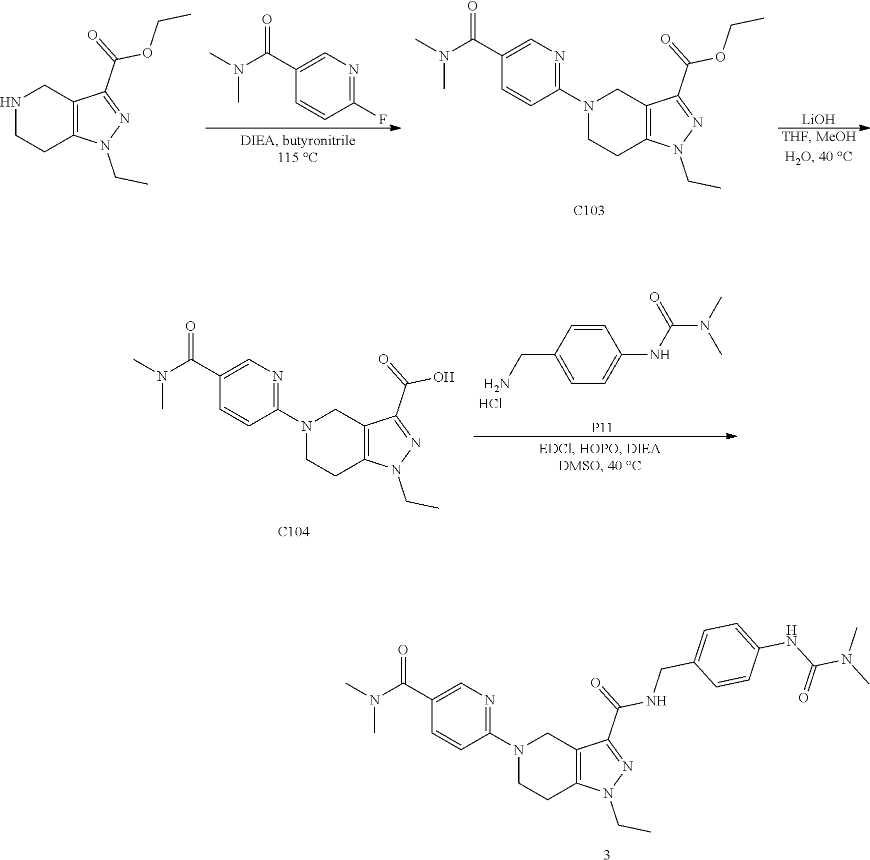

### Step 1. Preparation of ethyl 5-(5-(dimethylcarbamoyl)pyridin-2-yl)-1-ethyl-4,5,6,7-tetrahydro-1H-pyrazolo[4,3-c]pyridine-3-carboxylate (C103)

To a mixture of ethyl 1-ethyl-4,5,6,7-tetrahydro-1H-pyrazolo[4,3-c]pyridine-3-carboxylate hydrochloride (prepared using similar methods to Preparation 1, 0.350 g, 1.35 mmol) in butyronitrile (5.0 mL) and DIEA (0.697 g, 5.39 mmol) was added 6-fluoro-N,N-dimethylnicotinamide (CAS: 1032251-82-3, 0.249 g, 1.48 mmol) and reaction heated at 115 °C for 17 h. The solution was concentrated in vacuo, then redissolved in DCM, and purified by column chromatography (silica gel, 0-5% MeOH: DCM) to provide C103 (0.209 g, 41.7% yield) as a yellow oil. (LC/MS) m/z (M+H)⁺=372.4. ¹H NMR (600 MHz, (CD₃)₂SO) δ (ppm): 8.23 (d, 1H), 7.64 (dd, 1H), 6.91 (d, 1H), 4.73 (s, 2H), 4.28 (q, 2H), 4.09 (q, 2H), 3.95 (t, 2H), 2.96 (s, 6H), 2.81 (t, 2H), 1.34-1.28 (m, 6H).

### Step 2. Preparation of 5-(5-(dimethylcarbamoyl)pyridin-2-yl)-1-ethyl-4,5,6,7-tetrahydro-1H-pyrazolo[4,3-c]pyridine-3-carboxylic acid (C104)

To C103 (0.250 g, 0.673 mmol) in THF (2.0 mL), H₂O (2.0 mL), MeOH (0.2 mL) was added LiOH (80.6 mg, 3.37 mmol) at 15 °C. The reaction was heated to 40 °C and stirred for 1 h. The reaction was concentrated in vacuo to remove the organic layer, and the aqueous layer was acidified to pH=3 by 2N HCl. The acidic aqueous phase was concentrated in vacuo then purified by reverse phase HPLC (C₁₈ 150 mm×30 mm×5 μm column, Mobile Phase A: H₂O/Mobile Phase B: ACN (0.05% formic acid), 0 to 32% of Mobile Phase B over 9 min, 100% then Mobile Phase B hold 2 min, flow rate: 30 mL/min) and lyophilized to provide C104 (0.150 g, 64.9% yield) as a white solid. (LC/MS) m/z (M+H)⁺=344.3. ¹H NMR (400 MHz, CD₃OD) δ (ppm): 8.31-8.25 (m, 1H), 7.68 (dd, 1H), 6.91 (d, 1H), 4.77 (s, 2H), 4.15 (q, 2H), 4.06 (t, 2H), 3.09 (s, 6H), 2.86 (t, 2H), 1.40 (t, 3H).

### Step 3. Preparation of 5-(5-(dimethylcarbamoyl)pyridin-2-yl)-N-(4-(3,3-dimethylureido)benzyl)-1-ethyl-4,5,6,7-tetrahydro-1H-pyrazolo[4,3-c]pyridine-3-carboxamide (3)

The reaction was conducted in two batches and then combined for purification. To a solution of C104 (0.100 g, 0.291 mmol) in DMSO (3.0 mL) was added HOPO (97.1 mg, 0.874 mmol), DIEA (0.188 g, 1.46 mmol), and EDCI (0.134 g, 0.699 mmol) to form the first batch. The reaction mixture of the first batch was stirred at 40 °C for 15 min before P11 (73.6 mg, 0.320 mmol) was added. The suspension of the first batch was stirred at 40 °C for 2 h.

The reaction mixture of the first batch was combined with a second batch of the same reaction utilizing C104 (0.563 g, 1.64 mmol). The combined reaction was purified by reverse phase HPLC (C₁₈ 150 mm×40 mm×5 μm column, Mobile Phase A: H₂O/Mobile Phase B: ACN (NH₄OH—NH₄HCO₃), 3 to 43% of Mobile Phase B over 9 min, 100% of Mobile Phase B hold 2 min, flow rate: 60 mL/min) and lyophilized to provide 3 (0.467 g, 46.7% yield). (LC/MS) m/z (M+H)⁺=519.3. ¹H NMR (400 MHz, CDCl₃) δ (ppm): 8.32 (d, 1H), 7.62 (dd, 1H), 7.35 (d, 2H), 7.31-7.27 (m, 2H), 7.09 (t, 1H), 6.77 (d, 1H), 6.31 (s, 1H), 4.75 (s, 2H), 4.55 (d, 2H), 4.15 (t, 2H), 4.03 (q, 2H), 3.08 (s, 6H), 3.03 (s, 6H), 2.81-2.74 (m, 2H), 1.40 (t, 3H).

### Example 4

### 5-(4-(Dimethylcarbamoyl)phenyl)-1-methyl-N-(4-(methylcarbamoyl)benzyl)-4,5,6,7-tetrahydro-1H-pyrazolo[4,3-c]pyridine-3-carboxamide (4)

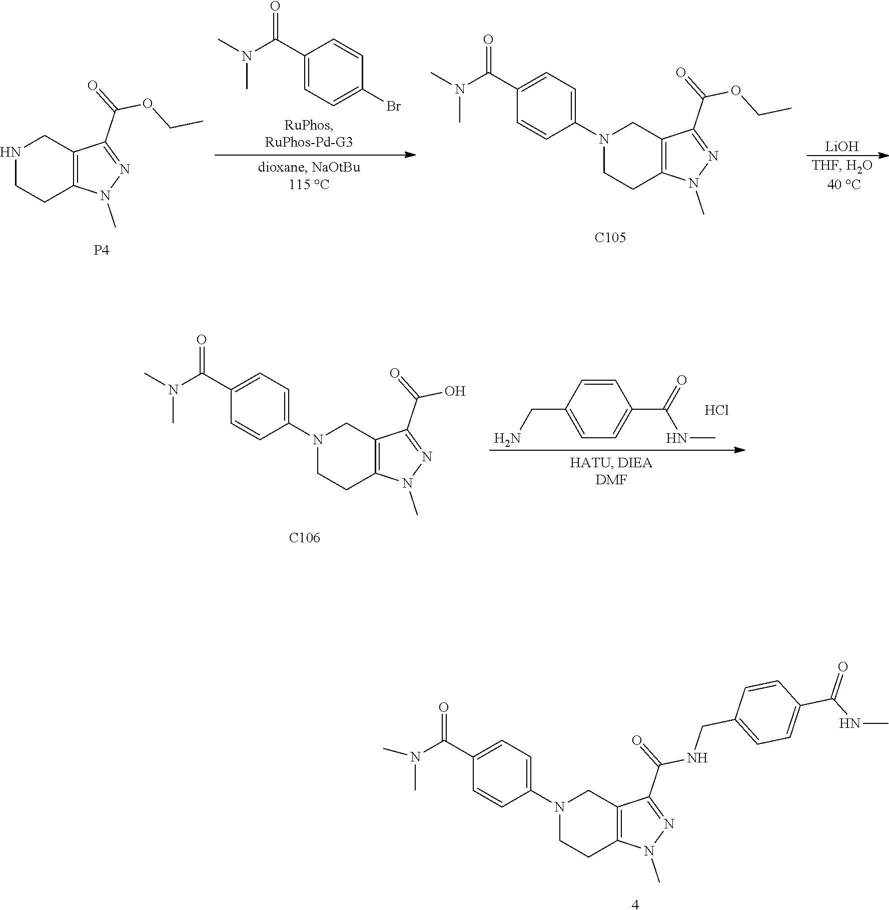

### Step 1. Preparation of ethyl 5-(4-(dimethylcarbamoyl)phenyl)-1-methyl-4,5,6,7-tetrahydro-1H-pyrazolo[4,3-c]pyridine-3-carboxylate (C105)

The reaction was conducted in two batches and then combined for purification. To a solution of P4 (50.0 mg, 0.239 mmol) and 4-bromo-N,N-dimethylbenzamide (CAS: 18469-37-9, 54.5 mg, 0.239 mmol) in dioxane (3.0 mL) was added RuPhos (11.2 mg, 0.0239 mmol), RuPhos Pd G3 (20.0 mg, 0.0239 mmol) and NaOtBu (45.9 mg, 0.478 mmol) at room temperature to form the first batch. The reaction mixture of the first batch was degassed with nitrogen for 1 min and heated at 115 °C. for 20 h.

The first batch's reaction mixture was combined with the second batch of the same reaction utilizing P4 (0.400 g, 1.91 mmol). The combined reaction was purified by HPLC (C₁₈ 150 mm×30 mm×5 μm column, Mobile Phase A: H₂O/Mobile Phase B: ACN (0.05% NH₄OH—NH₄HCO₃), 6 to 46% of Mobile Phase B over 9 min, 100% of Mobile Phase B hold 2 min, flow rate: 30 mL/min) and lyophilized to provide C105 (50.0 mg, 6.53% yield) as a white solid. (LC/MS) m/z (M+H)⁺=357.2.

### Step 2. Preparation of 5-(4-(dimethylcarbamoyl)phenyl)-1-methyl-4,5,6,7-tetrahydro-1H-pyrazolo[4,3-c]pyridine-3-carboxylic acid hydrochloride (C106)

To a solution of C105 (50.0 mg, 0.140 mmol) in THF (3.0 mL) and H₂O (0.5 mL) was added LiOH (17.7 mg, 0.421 mmol) at room temperature and then stirred at 40 °C for 16 h. The reaction was concentrated in vacuo, washed with H₂O (2×10 mL), and then acidified with 2M HCl until pH=5-6; the resulting solid was filtered to provide C106 (46.0 mg, 89.8% yield) as a white solid. This was used in the next step without further purification. (LC/MS) m/z (M+H)⁺=329.2.

### Step 3. Preparation of 5-(4-(dimethylcarbamoyl)phenyl)-1-methyl-N-(4-(methylcarbamoyl)benzyl)-4,5,6,7-tetrahydro-1H-pyrazolo[4,3-c]pyridine-3-carboxamide (4)

To a solution of C106 (46.0 mg, 0.140 mmol) in DMF (5.0 mL) was added HATU (79.9 mg, 0.210 mmol), DIEA (54.3 mg, 0.420 mmol), and 4-(aminomethyl)-N-methylbenzamide hydrochloride (CAS: 1158467-80-1, 28.1 mg, 0.140 mmol). The reaction was stirred at room temperature for 12 h. The residue was purified by reverse phase HPLC (C₁₈ 150 mm×30 mm×5 μm, Mobile Phase A: H₂O/Mobile Phase B: ACN (0.05% formic acid), 8 to 33% of Mobile Phase B over 9 min, 100% of Mobile Phase B hold 2 min, flow rate: 30 mL/min) and lyophilized to provide an impure 4 (25.0 mg) as a white solid. The solid was re-purified by reverse phase HPLC (C₁₈ 150 mm×30 mm×5 μm, Mobile Phase A: H₂O/Mobile Phase B: ACN (0.05% NH₄OH—NH₄HCO₃), 4 to 44% of Mobile Phase B over 9 min, 100% of Mobile Phase B hold 2 min, flow rate: 30 mL/min) and lyophilized to provide 4 (12.8 mg, 19.3% yield) as a white solid. (LC/MS) m/z (M+H)⁺=475.2. ¹H NMR (400 MHz, CD₃OD) δ (ppm): 7.80-7.74 (m, 2H), 7.46-7.42 (m, 2H), 7.38-7.33 (m, 2H), 7.08-7.02 (m, 2H), 4.60 (s, 2H), 4.50 (s, 2H), 3.80 (s, 3H), 3.74 (t, 2H), 3.06 (s, 6H), 2.91 (s, 3H), 2.84 (t, 2H).

### Example 5

### 7-(5-(Dimethylcarbamoyl)pyridin-2-yl)-N-(4-(3,3-dimethylureido)benzyl)-3-methyl-5,6,7,8-tetrahydroimidazo[1,5-a]pyrazine-1-carboxamide (5)

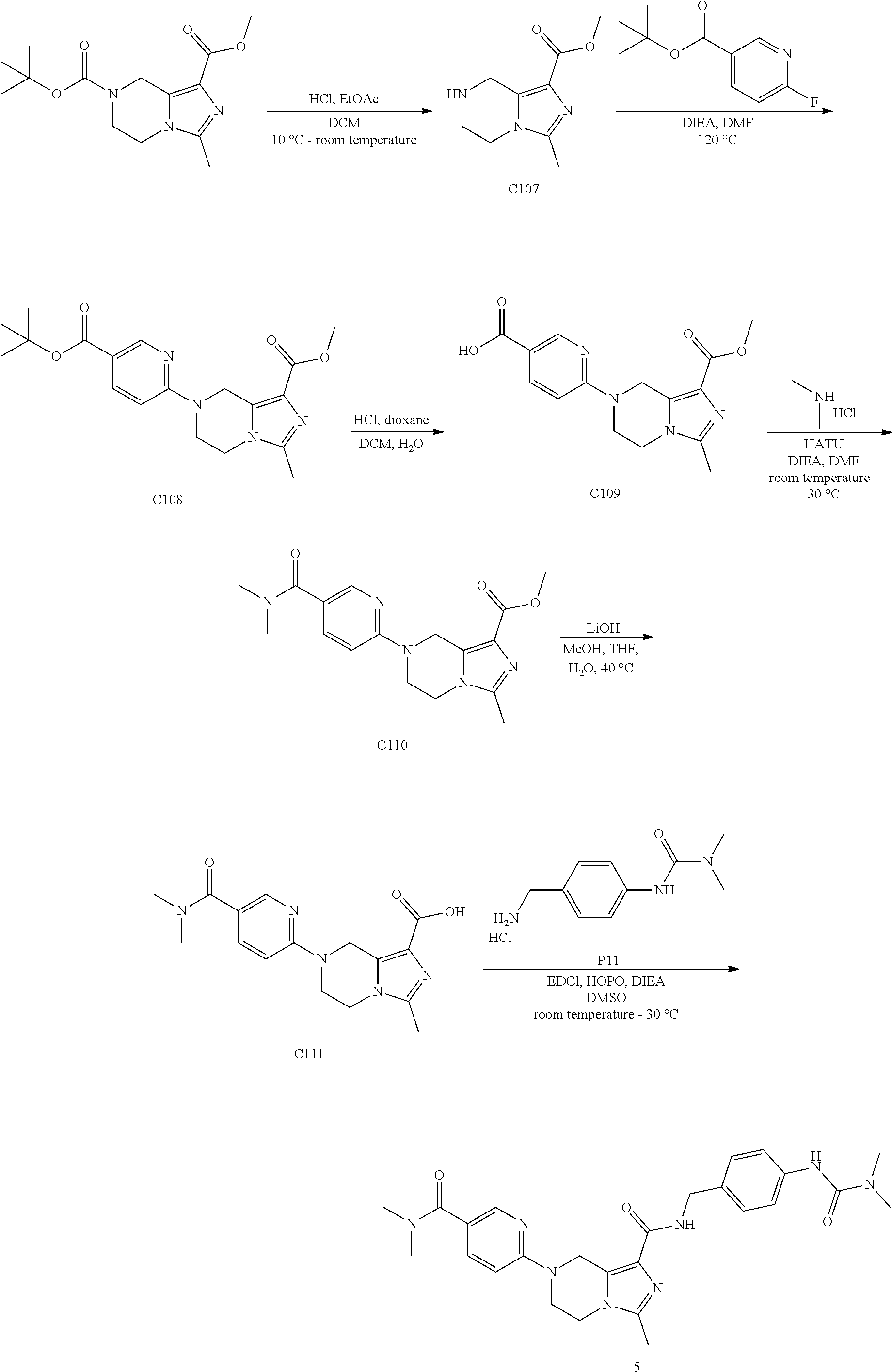

### Step 1. Preparation of methyl 3-methyl-5,6,7,8-tetrahydroimidazo[1,5-a]pyrazine-1-carboxylate hydrochloride (C107)

To a solution of 7-(tert-butyl) 1-methyl 3-methyl-5,6-dihydroimidazo[1,5-a]pyrazine-1,7(8H)-dicarboxylate (CAS: 1359655-89-2, 0.190 g, 0.643 mmol) in DCM (5.0 mL) was added 2M HCl in EtOAc (5.0 mL) at 10 °C. The reaction mixture was stirred at room temperature for 16 h. The resulting white suspension was concentrated in vacuo to provide the C107 (0.149 g, crude) as a white solid. This was used in the next step without further purification. ¹H NMR (400 MHz, CD₃OD) δ (ppm): 4.80 (s, 2H), 4.51 (t, 2H), 4.00 (s, 3H), 3.84 (t, 2H), 2.71 (s, 3H).

### Step 2. Preparation of methyl 7-(5-(tert-butoxycarbonyl)pyridin-2-yl)-3-methyl-5,6,7,8-tetrahydroimidazo[1,5-a]pyrazine-1-carboxylate (C108)

A mixture of C107 (90.0 mg, 0.388 mmol), tert-butyl 6-fluoronicotinate (CAS: 676560-01-3, 152 mg, 0.777 mmol), and DIEA (251 mg, 1.94 mmol) in DMF (4.0 mL) was stirred at 120 °C for 5 h. The light brown reaction mixture was concentrated in vacuo, and the residue was purified by column chromatography (silica gel, 0-100% EtOAc: PE) to provide C108 (87.0 mg, 60.0% yield) as an off-white solid. (LC/MS) m/z (M+H)⁺=373.1. ¹H NMR (400 MHz, CDCl₃) δ (ppm): 8.80-8.78 (m, 1H), 8.08 (dd, 1H), 6.75 (d, 1H), 5.00 (s, 2H), 4.31 (t, 2H), 4.02 (t, 2H), 3.93 (s, 3H), 2.43 (s, 3H), 1.58 (s, 9H).

### Step 3. Preparation of 6-(1-(methoxycarbonyl)-3-methyl-5,6-dihydroimidazo[1,5-a]pyrazin-7(8H)-yl)nicotinic acid hydrochloride (C109)

To a solution of C108 (87.0 mg, 0.234 mmol) in DCM (2.0 mL) and 2M HCl in dioxane (2.0 mL) was added H₂O (0.1 mL), then the reaction was stirred for 6 h at room temperature. The light-yellow reaction was concentrated in vacuo and then dried under high vacuum for 3 h to provide C109 (73.9 mg, crude) as a light brown residue. This was used in the next step without further purification. (LC/MS) m/z (M+H)⁺=317.1.

### Step 4. Preparation of methyl 7-(5-(dimethylcarbamoyl)pyridin-2-yl)-3-methyl-5,6,7,8-tetrahydroimidazo[1,5-a]pyrazine-1-carboxylate (C110)

To a solution of C109 (73.0 mg, 0.231 mmol) in DMF (5.0 mL) was added HATU (0.132 g, 0.346 mmol), DIEA (0.149 g, 1.15 mmol), and dimethylamine hydrochloride (CAS: 506-59-2; 28.2 mg, 0.346 mmol) at room temperature. The mixture was stirred at room temperature for 4 h, then heated to 30 °C and stirred for 16 h. The reaction mixture was concentrated in vacuo then the residue was purified by column chromatography (silica gel, 0-100% PE: EtOAc to 3.3% MeOH: DCM to provide C110 (71.0 mg, 78.9% yield) as an off-white solid.

### Step 5. Preparation of 7-(5-(dimethylcarbamoyl)pyridin-2-yl)-3-methyl-5,6,7,8-tetrahydroimidazo[1,5-a]pyrazine-1-carboxylic acid (C111)

To a solution of C110 (71.0 mg, 0.207 mmol) in THF (5.0 mL), MeOH (0.5 mL), H₂O (1.0 mL) was added LiOH (15.5 mg, 0.369 mmol) at room temperature. The reaction was stirred at 40 °C for 4 h and then acidified with formic acid to pH=5. The resulting mixture was concentrated in vacuo and then purified by reverse phase HPLC (C₁₈ 150 mm×30 mm×5 μm column, Mobile Phase A: H₂O/Mobile Phase B: ACN (0.05% formic acid), 0 to 16% of Mobile Phase B over 9 min, 100% of Mobile Phase B hold 2 min, flow rate: 30 mL/min) and lyophilized to provide C111 (41.0 mg, 60.2% yield) as a white solid. (LC/MS) m/z (M+H)⁺=330.1.

### Step 6. Preparation of 7-(5-(dimethylcarbamoyl)pyridin-2-yl)-N-(4-(3,3-dimethylureido)benzyl)-3-methyl-5,6,7,8-tetrahydroimidazo[1,5-a]pyrazine-1-carboxamide (5)

A mixture of C111 (40.0 mg, 0.121 mmol), HOPO (40.5 mg, 0.364 mmol), EDCI (55.9 mg, 0.291 mmol), and DIEA (78.5 mg, 0.607 mmol) in DMSO (2.0 mL) was stirred at room temperature for 20 min. P11 (30.7 mg, 0.134 mmol) was added, and the light brown reaction mixture was stirred at 30 °C for 3 h and 30 min. The residue was purified by reverse phase HPLC (C₁₈ 150 mm×30 mm×5 μm column, Mobile Phase A: H₂O/Mobile Phase B: ACN (0.05% formic acid), 0 to 36% of Mobile Phase B over 9 min, 100% of Mobile Phase B hold 2 min, flow rate: 30 mL/min) and lyophilized to provide 5 (28.4 mg, 46.4% yield) as a white solid. (LC/MS) m/z (M+H)⁺=505.4. ¹H NMR (400 MHz, (CD₃)₂SO) δ (ppm): 8.29-8.26 (m, 1H), 8.21-8.15 (m, 2H), 7.69 (dd, 1H), 7.40-7.35 (m, 2H), 7.17-7.13 (m, 2H), 6.95 (d, 1H), 5.02 (s, 2H), 4.32 (d, 2H), 4.09-3.98 (m, 4H), 2.97 (s, 6H), 2.90 (s, 6H), 2.28 (s, 3H).

### Example 6

### rac-(R)-5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (6)

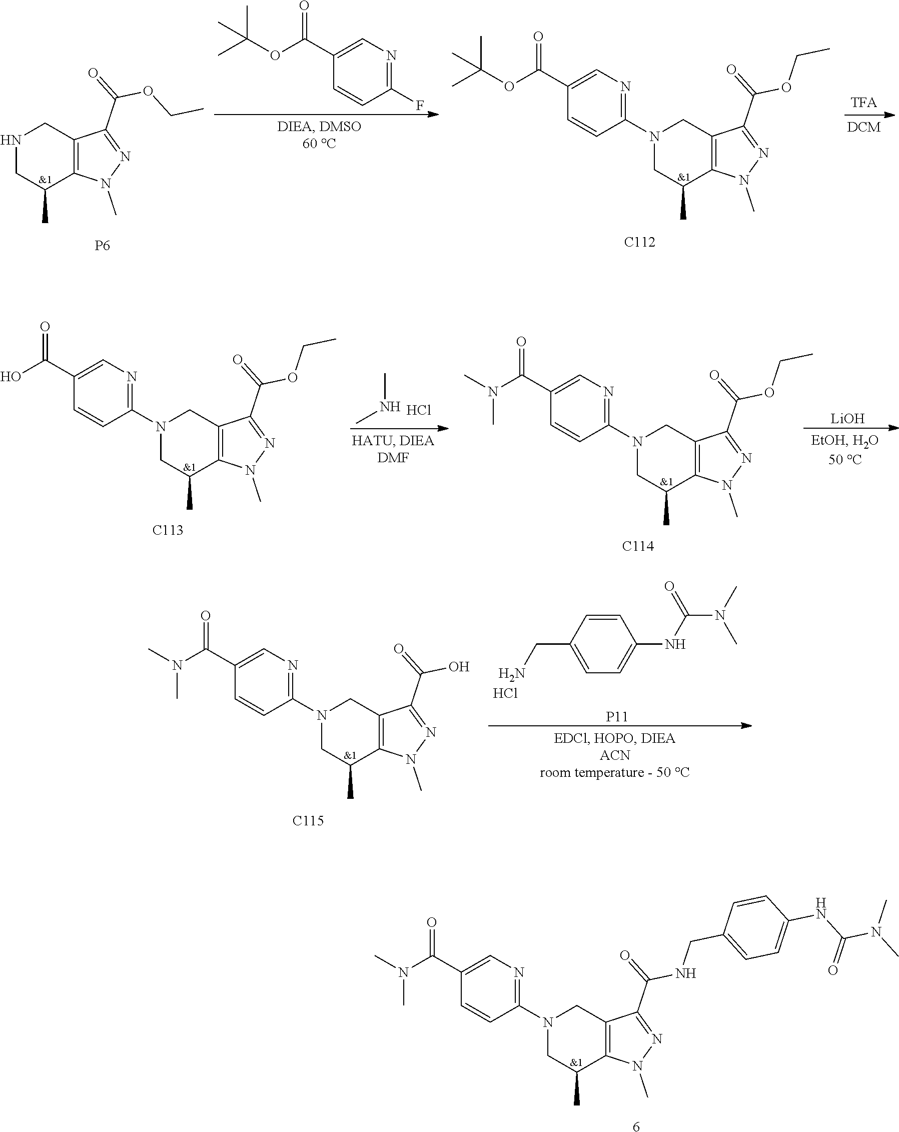

### Step 1: Preparation of rac-ethyl (R)-5-(5-(tert-butoxycarbonyl)pyridin-2-yl)-1,7-dimethyl-4,5,6,7-tetrahydro-1H-pyrazolo[4,3-c]pyridine-3-carboxylate (C112)

To a solution of P6 (0.426 g, 1.64 mmol), tert-butyl 6-fluoronicotinate (CAS: 676560-01-3, 0.356 g, 1.80 mmol) in DMSO (3.4 mL) was added DIEA (1.06 g, 8.20 mmol). The reaction mixture was stirred at 60 °C for 16 h, poured into H₂O (60 mL), and extracted with EtOAc (2×10 mL). The combined organic layers were washed with brine (2×10 mL), dried over Na₂SO₄, filtered, and concentrated in vacuo to give a residue. The residue was purified by column chromatography (silica gel, 0-100% EtOAc: heptane) to provide C112 (0.566 g, 86.2% yield) as a white crystal solid. (LC/MS) m/z (M+H)⁺=401.40. ¹H NMR (400 MHz, CDCl₃) δ (ppm): 8.77 (dd, 1H), 8.01 (dd, 1H), 6.69 (dd, 1H), 4.95 (d, 1H), 4.72 (dd, 1H), 4.50-4.36 (m, 3H), 3.89 (s, 3H), 3.46 (dd, 1H), 3.13-3.05 (m, 1H), 1.57 (s, 9H), 1.43 (t, 3H), 1.28 (d, 3H).

### Step 2: Preparation of rac-(R)-6-(3-(ethoxycarbonyl)-1,7-dimethyl-1,4,6,7-tetrahydro-5H-pyrazolo[4,3-c]pyridin-5-yl)nicotinic acid (C113)

TFA (2.81 g, 24.7 mmol) was added to a stirred solution of C112 (0.353 g, 0.881 mmol) in DCM (8.0 mL). The reaction was stirred at room temperature for 4 h and then concentrated in vacuo. The resulting gum was sonicated with heptane to yield a solid. The solvent was concentrated in vacuo then the white solid was dissolved in EtOAc and concentrated in vacuo to provide C113 (0.304 g, crude) as an off-white solid. This was used in the next step without further purification. (LC/MS) m/z (M+H)⁺=345.40.

### Step 3: Preparation of rac-ethyl (R)-5-(5-(dimethylcarbamoyl)pyridin-2-yl)-1,7-dimethyl-4,5,6,7-tetrahydro-1H-pyrazolo[4,3-c]pyridine-3-carboxylate (C114)

A mixture of C113 (0.304 g, 0.883 mmol), dimethylamine hydrochloride (CAS: 506-59-2; 86.4 mg, 1.06 mmol), HATU (0.369 g, 0.971 mmol), and DIEA (0.342 g, 2.65 mmol) in DMF (1.2 mL) was stirred at room temperature for 4 h. The reaction mixture was quenched with H₂O (30 mL) and extracted with MeOH in DCM (1:9; 4×100 mL). The combined organic layer was dried over Na₂SO₄ and then concentrated in vacuo. The residue was purified twice by column chromatography. The first purification was by column chromatography (silica gel, 0-100% EtOAc: heptane), and the second purification was by column chromatography (silica gel, 0-15% MeOH: DCM) to provide C114 (0.208 g, 63.4% yield) as a white solid. (LC/MS) m/z (M+H)⁺=372.40. ¹H NMR (400 MHz, CDCl₃) δ (ppm): 8.32-8.30 (m, 1H), 7.65 (dd, 1H), 6.74 (d, 1H), 4.92 (d, 1H), 4.58 (dd, 1H), 4.46-4.35 (m, 3H), 3.90-3.86 (m, 3H), 3.74-3.64 (m, 1H), 3.46 (dd, 1H), 3.09 (s, 6H), 1.44-1.38 (m, 3H), 1.29 (d, 3H).

### Step 4: Preparation of rac-(R)-5-(5-(dimethylcarbamoyl)pyridin-2-yl)-1,7-dimethyl-4,5,6,7-tetrahydro-1H-pyrazolo[4,3-c]pyridine-3-carboxylic acid (C115)

A flask was charged with C114 (0.106 g, 0.285 mmol), EtOH (2.3 mL), H₂O (0.9 mL), and LiOH (85.5 mg, 1.14 mmol) and then heated to 50 °C. The reaction mixture became a solution and was stirred at 50 °C for 1 h and 40 min. The reaction was acidified with 12.1M HCl (0.123 g, 3.37 mmol) then concentrated in vacuo and dried further under high vacuum to provide C115 (0.110 g, crude) as a white solid. This was used in the next step without further purification. (LC/MS) m/z (M+H)⁺=344.40.

### Step 5: Preparation of rac-(R)-5-(5-(dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (6)

To a solution of C115 (0.110 g, 0.348 mmol) in ACN (1.7 mL) was added HOPO (46.4 mg, 0.417 mmol) and EDCI (0.233 g, 1.22 mmol). The reaction was heated to 50 °C and stirred for 2 h and 20 min. The solution was cooled to room temperature, and DIEA (0.180 g, 1.39 mmol) was added to give a clear yellow solution. P11 (87.9 mg, 0.383 mmol) was added to the reaction and stirred for 1 h at room temperature. The reaction was concentrated in vacuo to give an oil, then dissolved in DCM for purification by column chromatography (silica gel, 0-20% MeOH: DCM) to give an impure 6 (65.0 mg) as a yellow oil. The yellow oil was purified again by reverse phase HPLC (Prep PG-45 MOD10 column, Mobile Phase A: H₂O with 2% ACN/Mobile Phase B: ACN, 5 to 60% of Mobile Phase B over 20 min, 95% of Mobile Phase B hold 5 min, flow rate: 30 mL/min) to provide 6 (34.6 mg, 19.2% yield). (LC/MS) m/z (M+H)⁺=519.2. ¹H NMR (400 MHz, CDCl₃) δ (ppm): 8.31 (d, 1H), 7.62 (dd, 1H), 7.37-7.32 (m, 2H), 7.28-7.24 (m, 2H), 7.08 (t, 1H), 6.76 (d, 1H), 6.39 (s, 1H), 4.93 (d, 1H), 4.65-4.43 (m, 5H), 3.79 (s, 3H), 3.47 (dd, 1H), 3.08 (s, 6H), 3.02 (s, 6H), 1.28 (d, 3H).

### Examples 7 and 8

### rel-(R or S)-5-(5-(Dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (7) and (8)

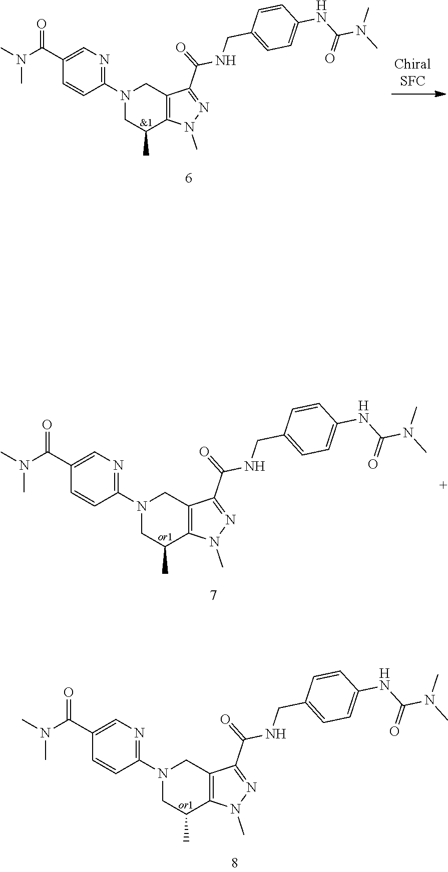

### Step 1. Preparation of rel-(R or S)-5-(5-(dimethylcarbamoyl)pyridin-2-yl)-N-(4-(3,3-dimethylureido)benzyl)-1,7-dimethyl-4,5,6,7-tetrahydro-1H-pyrazolo[4,3-c]pyridine-3-carboxamide (7) and (8)

Chiral SFC purification of compound 6 (44 mg, 0.085 mmol) [Column: Phenomenex Cellulose-1 250 mm×10 mm×5 μm; Mobile phase: 60% CO₂/40% (1:1 MeOH/ACN); flow rate: 25 mL/min; backpressure: 100 bar; column temperature: 40 °C.]provided the first eluting isomer as 7 (4.887 min, 22 mg, 50% yield, 100% ee): (LC/MS) m/z (M+H)⁺=519.3 and the second eluting isomer as 8 (5.377 min, 22 mg, 50% yield, 100% ee): (LC/MS) m/z (M+H)⁺=519.4.

### Example 9

### rac-(R)-7-(5-(Dimethylcarbamoyl)pyridin-2-yl)-N-(4-(3,3-dimethylureido)benzyl)-3,5-dimethyl-5,6,7,8-tetrahydroimidazo[1,5-a]pyrazine-1-carboxamide formate (9)

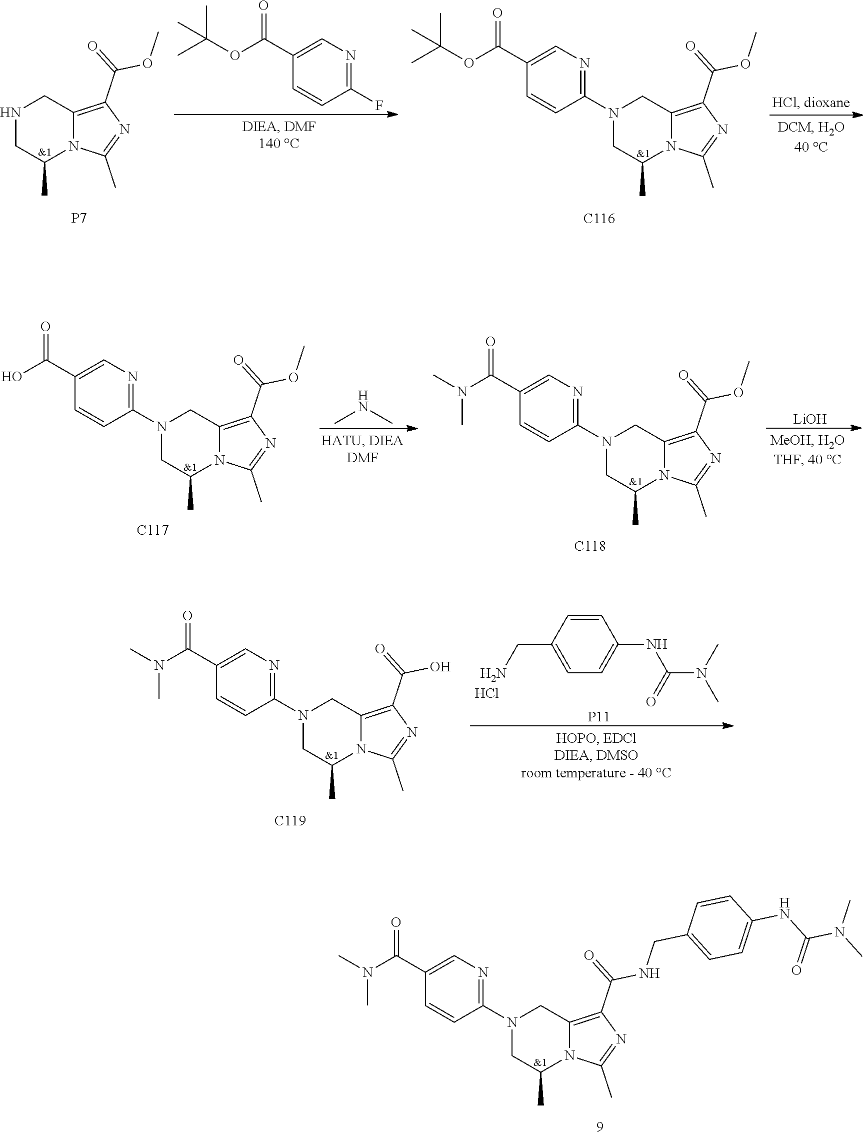

### Step 1: Preparation of rac-methyl (R)-7-(5-(tert-butoxycarbonyl)pyridin-2-yl)-3,5-dimethyl-5,6,7,8-tetrahydroimidazo[1,5-a]pyrazine-1-carboxylate (C116)

A mixture of P7 (0.111 g, 0.452 mmol), tert-butyl 6-fluoronicotinate (CAS: 676560-01-3, 0.223 g, 1.13 mmol), and DIEA (0.292 g, 2.26 mmol) in DMF (5.0 mL) was stirred at 140 °C for 2 h. The light brown reaction mixture was concentrated in vacuo. The residue was purified by column chromatography (silica gel, 0-100% EtOAc: PE then 2% MeOH: EtOAc) to provide C116 (45.0 mg, 25.7% yield) as a light brown solid. (LC/MS) m/z (M+H)⁺=387.1.

### Step 2. Preparation of rac-(R)-6-(1-(methoxycarbonyl)-3,5-dimethyl-5,6-dihydroimidazo[1,5-a]pyrazin-7(8H)-yl)nicotinic acid hydrochloride (C117)

To a solution of C116 (45.0 mg, 0.116 mmol) in DCM (3.0 mL) and 2M HCl in dioxane (10.0 mL) was added H₂O (0.02 mL) at room temperature. The reaction was stirred at 40 °C for 2 h and then concentrated in vacuo to provide C117 (42.7 mg, crude) as an off-white solid. This was used in the next step without further purification. (LC/MS) m/z (M+H)⁺=331.1.

### Step 3. Preparation of rac-methyl (R)-7-(5-(dimethylcarbamoyl)pyridin-2-yl)-3,5-dimethyl-5,6,7,8-tetrahydroimidazo[1,5-a]pyrazine-1-carboxylate (C118)

The reaction was conducted in three batches and then combined for purification. To a solution of C117 (53.0 mg, 0.144 mmol) in DMF (3.0 mL) was added HATU (82.4 mg, 0.217 mmol), DIEA (93.4 mg, 0.722 mmol) and dimethylamine hydrochloride (CAS: 506-59-2; 17.7 mg, 0.217 mmol) at room temperature and then heated to 40 °C and stirred for 16 h. The light brown reaction of the first batch was combined with the second batch of the same reaction utilizing C117 (42.0 mg, 0.116 mmol) and the third batch of the same reaction utilizing C117 (70.0 mg, 0.191 mmol). The combined reaction was concentrated in vacuo then purified by column chromatography (silica gel; 0-100% EtOAc: PE then 2.5% MeOH: EtOAc) to provide C118 (75.0 mg, 41.9% yield) as a light brown solid. (LC/MS) m/z (M+H)⁺=358.1.

### Step 4. Preparation of rac-(R)-7-(5-(dimethylcarbamoyl)pyridin-2-yl)-3,5-dimethyl-5,6,7,8-tetrahydroimidazo[1,5-a]pyrazine-1-carboxylic acid (C119)

The reaction was conducted in two batches and then combined for purification. To a solution of C118 (75.0 mg, 0.210 mmol) in THF (4.0 mL), MeOH (1.0 mL), and H₂O (2.0 mL) was added LiOH (17.6 mg, 0.420 mmol) at room temperature, which was then heated at 40 °C for 2 h. Another portion of LiOH (8.8 mg, 0.21 mmol) was added and stirred at 40 °C for 3 h. The reaction of the first batch was combined with the second batch of the same reaction utilizing C118 (33.0 mg, 0.0923 mmol), then acidified with formic acid to pH=−5 and concentrated in vacuo. The residue was purified by reverse phase HPLC (C₁₈ 150 mm×30 mm×5 μm column, Mobile Phase A: H₂O/Mobile Phase B: ACN (0.05% formic acid), 0 to 25% of Mobile Phase B over 9 min, 100% of Mobile Phase B hold 2 min, flow rate: 30 mL/min) and lyophilized to provide C119 (59.0 mg, 56.7% yield) as a light brown solid. (LC/MS) m/z (M+H)⁺=344.1.

### Step 5. Preparation of rac-(R)-7-(5-(dimethylcarbamoyl)pyridin-2-yl)-N-(4-(3,3-dimethylureido)benzyl)-3,5-dimethyl-5,6,7,8-tetrahydroimidazo[1,5-a]pyrazine-1-carboxamide formate (9)

A mixture of C119 (59.0 mg, 0.172 mmol), HOPO (57.3 mg, 0.515 mmol), EDCI (79.1 mg, 0.412 mmol) and DIEA (111 mg, 0.859 mmol) in DMSO (3.0 mL) was stirred for 30 min at 40 °C, then P11 (59.2 mg, 0.258 mmol) was added at room temperature. The light brown reaction mixture was stirred at 40 °C for 3 h and 30 min then the residue was purified by reverse phase HPLC (C₁₈ 150 mm×30 mm×5 μm column, Mobile Phase A: H₂O/Mobile Phase B: ACN (0.05% formic acid), 0 to 39% of Mobile Phase B over 9 min, 100% of Mobile Phase B hold 2 min, flow rate: 30 mL/min) and lyophilized to provide 9 (58.2 mg, 43.2% yield) as a white solid. (LC/MS) m/z (M+H)⁺=519.5. ¹H NMR (400 MHz, CD₃OD) δ (ppm): 8.31 (d, 1H), 7.74 (dd, 1H), 7.36-7.30 (m, 2H), 7.26 (d, 2H), 6.93 (d, 1H), 5.39 (d, 1H), 4.67-4.41 (m, 5H), 3.41 (dd, 1H), 3.10 (s, 6H), 3.04-2.99 (m, 6H), 2.41 (s, 3H), 1.40 (d, 3H).

### Example 10

### 1-Cyclopropyl-5-(5-(dimethylcarbamoyl)pyridin-2-yl)-N-(4-(3,3-dimethylureido)benzyl)-4,5,6,7-tetrahydro-1H-pyrazolo[4,3-c]pyridine-3-carboxamide trifluoroacetate (10)

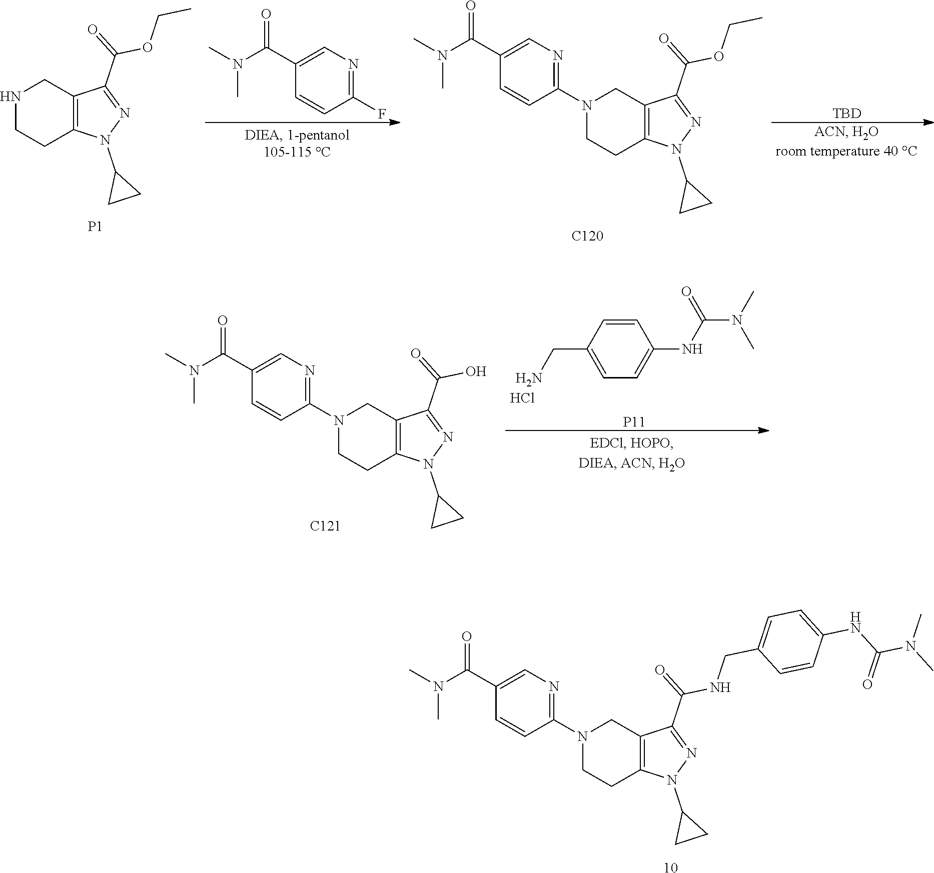

### Step 1. Preparation of ethyl 1-cyclopropyl-5-(5-(dimethylcarbamoyl)pyridin-2-yl)-4,5,6,7-tetrahydro-1H-pyrazolo[4,3-c]pyridine-3-carboxylate (C120)

To a solution of P1 (0.070 g, 0.26 mmol) in 1-pentanol (1 mL) and DIEA (0.20 g, 1.5 mmol) was added 6-fluoro-N,N-dimethylnicotinamide (CAS: 1032251-82-3; 65 mg, 0.39 mmol). The reaction mixture was heated at 105 °C for 24 h, then the temperature was increased to 115 °C and stirred for 23 h. The reaction mixture was concentrated in vacuo and then dissolved with DCM. The solution was purified by column chromatography (silica gel, 0-5% MeOH: DCM) to provide C120 (0.10 g, crude) as a yellow oil. The oil was used directly in the next step without further purification.

### Step 2. Preparation of 1-cyclopropyl-5-(5-(dimethylcarbamoyl)pyridin-2-yl)-4,5,6,7-tetrahydro-1H-pyrazolo[4,3-c]pyridine-3-carboxylic acid (C121)

To a solution of C120 (99 mg, 0.26 mmol) in ACN (1 mL) and H₂O (93 μL) was added TBD (0.11 g, 0.77 mmol) and then stirred for 40 min at room temperature followed by 40 °C for 2 h. The reaction was then cooled to room temperature, and 12.1 M HCl (24 mg, 0.65 mmol) was added. The acidic reaction mixture was concentrated in vacuo to provide C121 (0.090 g, crude) as a yellow oil. The oil was used directly in the next step without further purification. (LC/MS) m/z (M+H)⁺=356.2.

### Step 3. Preparation of 1-cyclopropyl-5-(5-(dimethylcarbamoyl)pyridin-2-yl)-N-(4-(3,3-dimethylureido)benzyl)-4,5,6,7-tetrahydro-1H-pyrazolo[4,3-c]pyridine-3-carboxamide trifluoroacetate (10)

To a solution of C121 (0.090 g, 0.25 mmol) in ACN (1.0 mL) and H₂O (0.2 mL) was added HOPO (34 mg, 0.30 mmol) and EDCI (78 mg, 0.41 mmol). To the reaction mixture was added an additional portion of EDCI (78 mg, 0.41 mmol) and then was stirred at room temperature for 20 min before DIEA (98 mg, 0.76 mmol) and P11 (0.070 g, 0.30 mmol) were added. The suspension was stirred at room temperature for 30 min and then was concentrated in vacuo. The residue was diluted with DMSO then acidified with TFA and purified with reverse phase HPLC (Sunfire C₁₈ 100 mm×19 mm×5 μm column, Mobile Phase A: H₂O (0.05% TFA)/Mobile Phase B: ACN (0.05% TFA), 5 to 95% of Mobile Phase B over 9 min, 95% of Mobile Phase B hold 1 min, flow rate=25 mL/min) and lyophilized to provide 10 (24 mg, 18% yield). (LC/MS) m/z (M+H)⁺=531.5. ¹H NMR (600 MHz, (CD₃)₂SO) δ (ppm): 8.46 (t, 1H), 8.22-8.21 (m, 2H), 7.68 (dd, 1H), 7.40-7.34 (m, 2H), 7.15 (d, 2H), 6.96 (d, 1H), 4.75 (s, 2H), 4.32 (d, 2H), 3.96 (t, 2H), 3.56 (tt, 1H), 2.97 (s, 6H), 2.91-2.86 (m, 8H), 1.12-1.05 (m, 2H), 1.01-0.95 (m, 2H).

### Example 12

### N-(4-(3,3-Dimethylureido)benzyl)-1-methyl-5-(5-(1-methyl-1H-imidazol-2-yl)pyridin-2-yl)-4,5,6,7-tetrahydro-1H-pyrazolo[4,3-c]pyridine-3-carboxamide (12)

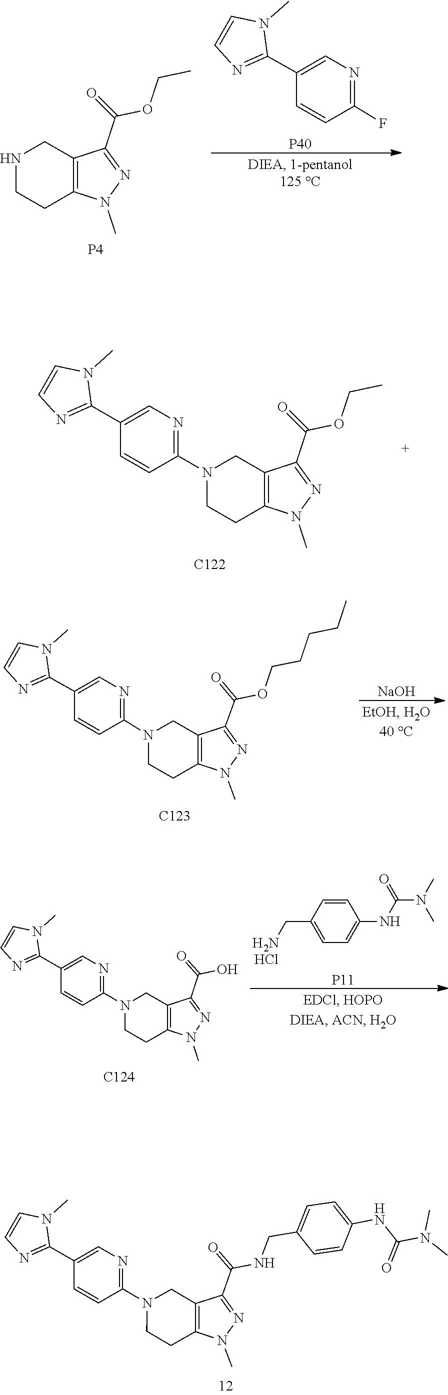

### Step 1. Preparation of ethyl 1-methyl-5-(5-(1-methyl-1H-imidazol-2-yl)pyridin-2-yl)-4,5,6,7-tetrahydro-1H-pyrazolo[4,3-c]pyridine-3-carboxylate (C122) and pentyl 1-methyl-5-(5-(1-methyl-1H-imidazol-2-yl)pyridin-2-yl)-4,5,6,7-tetrahydro-1H-pyrazolo[4,3-c]pyridine-3-carboxylate (C123)

To a suspension of P4 (0.60 g, 2.4 mmol) in 1-pentanol (5 mL) was added DIEA (1.9 g, 15 mmol) and P40 (0.47 g, 2.7 mmol), then heated at 125 °C for 20 h. The reaction mixture was checked by LCMS which showed 30% of C122 (LC/MS) m/z (M+H)⁺=367.3 and 32% of C123 at (LC/MS) m/z (M+H)⁺=409.3 was formed due to transesterification with 1-pentanol. The suspension was concentrated in vacuo then dissolved in DCM and heptane. The solution was purified by column chromatography (silica gel, 0-10% MeOH: DCM) to form a mixture of C122 and C123 (1.1 g, crude) as a brown oil. The brown oil was used directly in the next step without further purification.

### Step 2. Preparation of 1-methyl-5-(5-(1-methyl-1H-imidazol-2-yl)pyridin-2-yl)-4,5,6,7-tetrahydro-1H-pyrazolo[4,3-c]pyridine-3-carboxylic acid (C124)

To a reaction mixture of C122 and C123 (0.80 g, 2.2 mmol) in EtOH (6 mL) and H₂O (4 mL) was added NaOH (0.35 g, 8.7 mmol) then stirred at 40 °C for 50 min. The suspension was concentrated in vacuo and acidified with 12.1 M HCl (0.40 g, 11 mmol) to pH-1. The acidic reaction mixture was extracted with DCM then the organic layer was concentrated in vacuo to form C124 (0.30 g, crude) as a brown oil. The oil was used directly in the next step without further purification.

### Step 3. Preparation of N-(4-(3,3-dimethylureido)benzyl)-1-methyl-5-(5-(1-methyl-1H-imidazol-2-yl)pyridin-2-yl)-4,5,6,7-tetrahydro-1H-pyrazolo[4,3-c]pyridine-3-carboxamide (12)

To a reaction mixture of C124 (0.30 g, 0.89 mmol) in ACN (3.0 mL) and H₂O (0.5 mL) was added HOPO (0.12 g, 1.1 mmol) and EDCI (0.27 g, 1.4 mmol). The suspension was stirred at room temperature for 35 min before adding DIEA (0.34 g, 2.7 mmol) and P11 (0.24 g, 1.1 mmol). The reaction mixture was stirred at room temperature for 30 min and then was concentrated in vacuo. The residue was split into two portions for purification.

The first portion of the residue was diluted with DMSO and then purified with reverse- phase HPLC (Sunfire C₁₈ 100 mm×19 mm×5 μm column, Mobile Phase A: H₂O (0.05% TFA)/Mobile Phase B: ACN (0.05% TFA), 5 to 95% of Mobile Phase B over 9 min, 95% of Mobile Phase B hold 1 min, flow rate=25 mL/min) and lyophilized to provide 12 (8.5 mg).

The second portion of the residue was diluted with DCM and then purified by column chromatography (silica gel, 0-5% MeOH: DCM) to provide an impure residue. The impure residue was diluted with MeOH then purified by SFC (Princeton Methanesulfamide Column 250 mm×30 mm×5 μm, 70% CO₂/30% MeOH (0.2% 7N NH₃ in MeOH), backpressure=100 bar, temperature=40 °C, flow rate=80 mL/min) to provide 12 (62 mg) as a tan solid.

The two purified batches were combined to form 12 (0.070 g, 15% yield) as a solid. (LC/MS) m/z (M+H)⁺=514.4. ¹H NMR (600 MHz, (CD₃)₂SO) δ (ppm): 8.47 (t, 1H), 8.43 (d, 1H), 8.22 (s, 1H), 7.84 (dd, 1H), 7.42-7.34 (m, 2H), 7.22 (s, 1H), 7.16 (d, 2H), 7.01-6.93 (m, 2H), 4.77 (s, 2H), 4.32 (d, 2H), 3.96 (t, 2H), 3.76 (s, 3H), 3.71 (s, 3H), 2.90 (s, 6H), 2.80 (t, 2H).

### Example 18

### 5-(5-(Dimethylcarbamoyl)pyridin-2-yl)-N-(4-(3,3-dimethylureido)benzyl)-4,5,6,7-tetrahydro-1H-pyrazolo[4,3-c]pyridine-3-carboxamide (18)

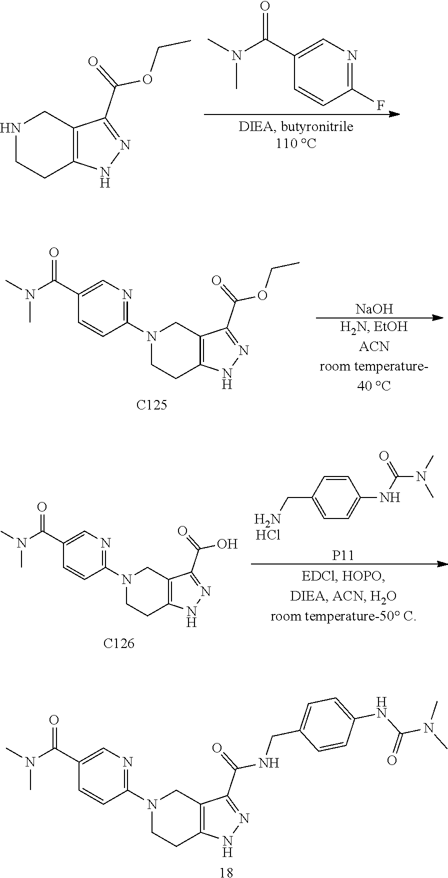

### Step 1. Preparation of ethyl 5-(5-(dimethylcarbamoyl)pyridin-2-yl)-4,5,6,7-tetrahydro-1H-pyrazolo[4,3-c]pyridine-3-carboxylate (C125)

To a reaction mixture of ethyl 4,5,6,7-tetrahydro-1H-pyrazolo[4,3-c]pyridine-3-carboxylate hydrochloride (CAS: 1211512-51-4; 0.380 g, 1.64 mmol) in butyronitrile (10 mL) and DIEA (0.848 g, 6.56 mmol) was added 6-fluoro-N,N-dimethylnicotinamide (CAS: 1032251-82-3, 0.290 g, 1.72 mmol) then stirred at 110 °C for 23 h. The solution was concentrated in vacuo and then redissolved in DCM. The solution was purified by column chromatography (silica gel, 0-7% MeOH: DCM) to provide C125 (0.439 g, 80.0% yield) as a yellow oil. (LC/MS) m/z (M+H)⁺=344.3.

### Step 2. Preparation of 5-(5-(dimethylcarbamoyl)pyridin-2-yl)-4,5,6,7-tetrahydro-1H-pyrazolo[4,3-c]pyridine-3-carboxylic acid (C126)

To a solution of C125 (0.439 g, 1.28 mmol) in ACN (5 mL), H₂O (2 mL), and EtOH (5 mL) was added NaOH (102 mg, 2.56 mmol). The solution was stirred at room temperature for 2 h then heated to 40 °C and stirred for 3 h and 20 min. The reaction solution was acidified with 12.1 M HCl (0.140 g, 3.84 mmol) then concentrated in vacuo to provide C126 (0.384 g, crude) as an off-white solid. The solid was used directly in the next step without further purification. (LC/MS) m/z (M+H)⁺=316.3.

### Step 3. Preparation of 5-(5-(dimethylcarbamoyl)pyridin-2-yl)-N-(4-(3,3-dimethylureido)benzyl)-4,5,6,7-tetrahydro-1H-pyrazolo[4,3-c]pyridine-3-carboxamide (18)

To a solution of C126 (0.200 g, 0.634 mmol) in ACN (5 mL) was added HOPO (84.6 mg, 0.761 mmol) and EDCI (0.195 g, 1.01 mmol). The reaction mixture was stirred at 50 °C for 20 min, then H₂O (1 mL), DIEA (0.246 g, 1.90 mmol), and P11 (0.160 g, 0.698 mmol) were added. The reaction mixture was stirred at room temperature for 15 min then concentrated in vacuo. The residue was dissolved in DCM and then purified by column chromatography (silica gel, 0-15% MeOH: DCM) to form an impure 18 (0.386 g).

A portion of the impure 18 (0.0400 g) was purified by reverse phase HPLC (Sunfire C₁₈ 100 mm×19 mm×5 μm column, Mobile Phase A: H₂O/Mobile Phase B: ACN (0.05% TFA), 5 to 95% of Mobile Phase B over 9 min, 95% oh Mobile Phase B hold 1 min, flow rate=25 mL/min) and lyophilized to provide 18 (9.90 mg).

The remaining of the portion of the impure 18 (0.346 g) was purified by SFC (Princeton CN 250 mm×30 mm×5 μm, 70% CO₂/25% MeOH (7N NH₃ in MeOH), backpressure=100 bar, temperature=40 °C, flow rate=80 mL/min) to provide 18 (55.9 mg).

The two final batches were combined to provide 18 (65.8 mg, 21.1% yield) as a solid. (LC/MS) m/z (M+H)⁺=491.4. ¹H NMR (400 MHz, CD₃OD) δ (ppm): 8.26 (d, 1H), 7.67 (dd, 1H), 7.35-7.25 (m, 4H), 6.89 (d, 1H), 4.78 (s, 2H), 4.50 (s, 2H), 4.05 (t, 2H), 3.08 (s, 6H), 3.00 (s, 6H), 2.84 (t, 2H).

### Example 23

### N-(4-(3,3-Dimethylureido)benzyl)-1-ethyl-5-(5-(1-methyl-1H-1,2,4-triazol-5-yl)pyridin-2-yl)-4,5,6,7-tetrahydro-1H-pyrazolo[4,3-c]pyridine-3-carboxamide (23)

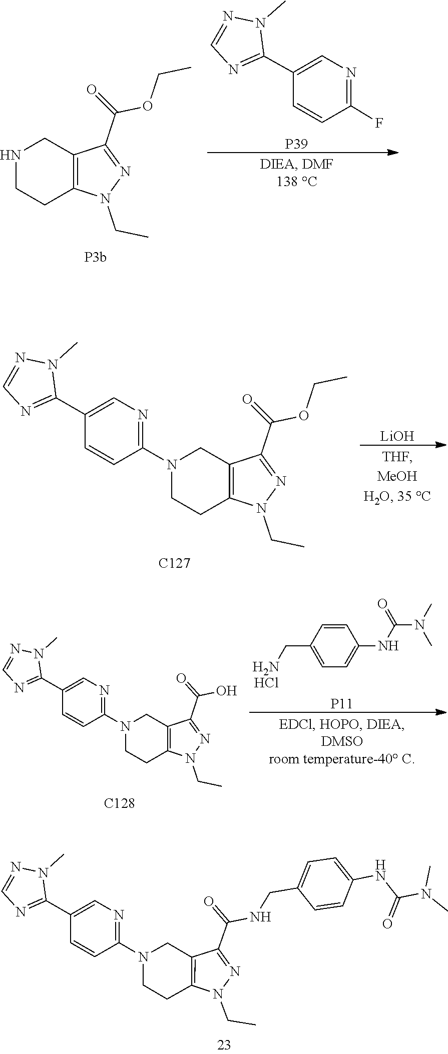

### Step 1. Preparation of ethyl 1-ethyl-5-(5-(1-methyl-1H-1,2,4-triazol-5-yl)pyridin-2-yl)-4,5,6,7-tetrahydro-1H-pyrazolo[4,3-c]pyridine-3-carboxylate (C127)

To a reaction mixture of P3b (0.150 g, 0.842 mmol) and P39 (0.219 g, 0.842 mmol) in DMF (4.2 mL) was added DIEA (0.544 g, 4.21 mmol) and then stirred at 138 °C for 48 h. The suspension was diluted with EtOAc (30 mL), and then the organic layer was washed with H₂O (20 mL) and brine (20 mL). The organic layer was dried with Na₂SO₄ and then concentrated in vacuo. The residue was purified by column chromatography (silica gel, 0-58% THF: PE) to provide C127 (0.200 g, 62.3% yield) as a white solid. (LC/MS) m/z (M+H)⁺=382.2.

### Step 2. Preparation of 1-ethyl-5-(5-(1-methyl-1H-1,2,4-triazol-5-yl)pyridin-2-yl)-4,5,6,7-tetrahydro-1H-pyrazolo[4,3-c]pyridine-3-carboxylic acid (C128)

To a solution of C127 (0.200 g, 0.524 mmol) in THF (2.0 mL), H₂O (0.5 mL), and MeOH (0.5 mL) was added LiOH (66.0 mg, 1.57 mmol) at room temperature. The reaction mixture was heated to 35 °C and stirred for 2 h. The suspension was extracted with EtOAc (2×10 mL), and then the combined organic layer was washed with H₂O (10 mL). The aqueous layer was acidified to pH=3 with citric acid, which caused precipitation to form. The suspension was filtered, and then the filter cake was collected and lyophilized to provide C128 (0.150 g, crude) as a white solid. The solid was used directly in the next step without further purification. (LC/MS) m/z (M+H)⁺=354.2.

### Step 3. Preparation of N-(4-(3,3-dimethylureido)benzyl)-1-ethyl-5-(5-(1-methyl-1H-1,2,4-triazol-5-yl)pyridin-2-yl)-4,5,6,7-tetrahydro-1H-pyrazolo[4,3-c]pyridine-3-carboxamide (23)

The reaction was conducted in two batches and then combined for purification. To a solution of C128 (0.060 g, 0.17 mmol) in DMSO (1.0 mL) was added HOPO (56 mg, 0.50 mmol), DIEA (0.11 g, 0.85 mmol), and EDCI (78 mg, 0.41 mmol). The reaction mixture was stirred at room temperature for 5 min, and then P11 (58 mg, 0.25 mmol) was added to form the first batch. The reaction mixture of the first batch was stirred at 40 °C for 2 h.

A second batch of the same reaction was conducted with C128 (15 mg, 0.043 mmol). The two batches were combined then purified by reverse phase HPLC (Boston Prime C₁₈ 150 mm×30 mm×5 μm column, Mobile Phase A: H₂O/Mobile Phase B: ACN (NH₄OH—NH₄HCO₃), 20 to 50% of Mobile Phase B over 9 min, 100% of Mobile Phase B hold 3 min, flow rate=50 mL/min) and lyophilized to provide 23 (62 mg, 55% yield) as a white solid. (LC/MS) m/z (M+H)⁺=529.3. ¹H NMR (400 MHz, (CD₃)₂SO) δ (ppm): 8.56 (d, 1H), 8.44 (t, 1H), 8.23 (s, 1H), 7.99-7.92 (m, 2H), 7.45-7.34 (m, 2H), 7.22-7.17 (m, 2H), 7.05 (d, 1H), 4.83 (s, 2H), 4.36 (d, 2H), 4.11 (q, 2H), 4.02 (t, 2H), 3.97 (s, 3H), 2.93 (s, 6H), 2.86 (t, 2H), 1.36 (t, 3H).

### Example 26

### rac-(R)-5-(4-(Dimethylcarbamoyl)phenyl)-N-(4-(3,3-dimethylureido)benzyl)-1-methyl-4,5,6,7-tetrahydro-1H-indazole-3-carboxamide (26)

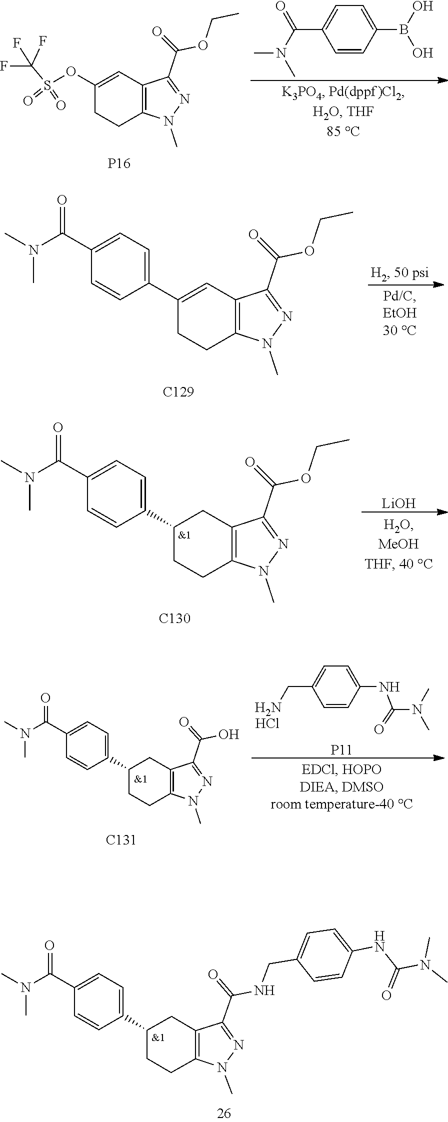

### Step 1. Preparation of ethyl 5-(4-(dimethylcarbamoyl)phenyl)-1-methyl-6,7-dihydro-1H-indazole-3-carboxylate (C129)

Under nitrogen gas, to a mixture of P16 (0.500 g, 1.41 mmol) and (4-(dimethylcarbamoyl)phenyl)boronic acid (0.409 g, 2.12 mmol) in H₂O (1 mL) and THF (10 mL) was added Pd(dppf)Cl₂ (0.103 g, 0.141 mmol) and K₃PO₄ (0.899 g, 4.23 mmol). The reaction mixture was stirred at 85 °C for 16 h and then concentrated in vacuo. The residue was purified by column chromatography (silica gel, 0-62% THF: PE) to provide C129 (0.470 g, 94.2% yield) as a pink solid. (LC/MS) m/z (M+H)⁺=354.1. ¹H NMR (400 MHz, (CD₃)₂SO) δ (ppm): 7.62-7.55 (m, 2H), 7.45-7.39 (m, 2H), 7.15-7.12 (m, 1H), 4.33-4.24 (m, 2H), 3.85-3.82 (m, 3H), 3.76-3.54 (m, 2H), 3.01-2.94 (m, 6H), 2.93-2.85 (m, 2H), 1.31 (t, 3H).

### Step 2. Preparation of rac-ethyl (R)-5-(4-(dimethylcarbamoyl)phenyl)-1-methyl-4,5,6,7-tetrahydro-1H-indazole-3-carboxylate (C130)

A solution of C129 (0.050 g, 0.14 mmol) was added in a thick-walled pressure vial in EtOH (5 mL). Pd/C (45 mg, 0.042 mmol) was added to the solution. The vial was evacuated under vacuum then purged with H₂ gas. The reaction mixture was stirred under H₂ atmosphere (50 psi) at 30 °C for 16 h then concentrated in vacuo to provide C130 (45 mg, 89% yield) as a yellow oil. ¹H NMR (400 MHz, CDCl₃) δ (ppm): 7.39-7.35 (m, 2H), 7.30-7.26 (m, 2H), 4.37 (q, 2H), 3.83 (s, 3H), 3.19 (dd, 1H), 3.14-2.98 (m, 6H), 2.97-2.90 (m, 1H), 2.81-2.61 (m, 3H), 2.20-2.12 (m, 1H), 2.05-1.92 (m, 1H), 1.37 (t, 3H).

### Step 3. Preparation of rac-(R)-5-(4-(dimethylcarbamoyl)phenyl)-1-methyl-4,5,6,7-tetrahydro-1H-indazole-3-carboxylic acid (C131)

To a solution of C130 (45 mg, 0.13 mmol) in THF (1.00 mL), H₂O (0.25 mL), and MeOH (0.25 mL) was added LiOH (16 mg, 0.38 mmol). The reaction mixture was stirred at 40 °C for 2 h, diluted with EtOAc (2×10 mL), and washed with H₂O (10 mL). The aqueous phase was separated and adjusted with citric acid to pH-3, then extracted with DCM (5×50 mL). The combined organic layer was dried with Na₂SO₄ and concentrated in vacuo to provide C131 (35 mg, 84% yield) as a brown solid. (LC/MS) m/z (M+H)⁺=328.1.

### Step 4. Preparation of rac-(R)-5-(4-(dimethylcarbamoyl)phenyl)-N-(4-(3,3-dimethylureido)benzyl)-1-methyl-4,5,6,7-tetrahydro-1H-indazole-3-carboxamide (26)

To a solution of C131 (35 mg, 0.11 mmol) in DMSO (1 mL) was added HOPO (35 mg, 0.31 mmol), EDCI (49 mg, 0.26 mmol), and DIEA (69 mg, 0.53 mmol). The reaction mixture was stirred at room temperature for 5 min, and then P11 (37 mg, 0.16 mmol) was added. The suspension was stirred at 40 °C for 1 h then was purified by reverse phase HPLC (C₁₈ 150 mm×30 mm×5 μm column, Mobile Phase A: H₂O (NH₄OH—NH₄HCO₃)/Mobile Phase B: ACN, 14 to 44% of Mobile Phase B over 10 min, 100% of Mobile Phase B hold 3 min, flow rate=50 mL/min) and lyophilized to provide 26 (11 mg, 20.% yield) as a white solid. (LC/MS) m/z (M+H)⁺=503.4. ¹H NMR (400 MHz, (CD₃)₂SO) δ (ppm): 8.31 (t, 1H), 8.22 (s, 1H), 7.42-7.32 (m, 6H), 7.17-7.11 (m, 2H), 4.31 (d, 2H), 3.76 (s, 3H), 3.14-3.04 (m, 1H), 3.00-2.89 (m, 12H), 2.85-2.58 (m, 4H), 2.12-1.88 (m, 2H).

### Example 30

### N-(4-(3,3-Dimethylureido)benzyl)-1-ethyl-5-(5-(1-methyl-1H-imidazol-2-yl)pyridin-2-yl)-4,5,6,7-tetrahydro-1H-pyrazolo[4,3-c]pyridine-3-carboxamide (30)

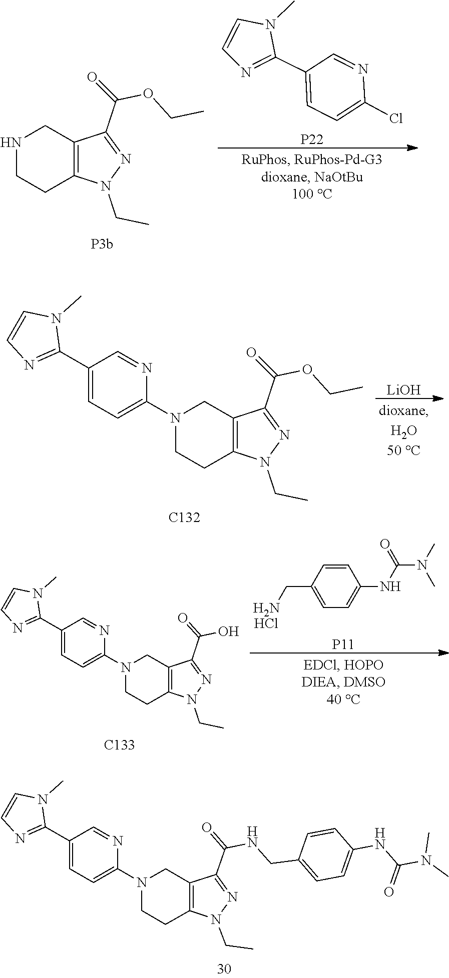

### Step 1. Preparation of ethyl 1-ethyl-5-(5-(1-methyl-1H-imidazol-2-yl)pyridin-2-yl)-4,5,6,7-tetrahydro-1H-pyrazolo[4,3-c]pyridine-3-carboxylate (C132)

To a solution of P3b (0.760 g, 2.93 mmol), P22 (0.680 g, 3.51 mmol) and NaOtBu (0.843 g, 8.78 mmol) in dioxane (15 mL) was added RuPhos (0.273 g, 0.585 mmol) and RuPhos Pd G3 (0.489 g, 0.585 mmol) at room temperature. The reaction mixture was degassed with nitrogen gas and then stirred at 100 °C for 4 h to provide C132 (1.11 g, crude) as a solution. The solution was used directly in the next step without further purification. (LC/MS) m/z (M-ethyl)+=353.2.

### Step 2. Preparation of 1-ethyl-5-(5-(1-methyl-1H-imidazol-2-yl)pyridin-2-yl)-4,5,6,7-tetrahydro-1H-pyrazolo[4,3-c]pyridine-3-carboxylic acid (C133)

To a solution of C132 (1.26 g, 3.31 mmol) in dioxane (15 mL) and H₂O (5 mL) was added LiOH (0.397 g, 16.6 mmol) at room temperature then stirred at 50 °C for 5 h. The reaction was purified by reverse phase HPLC (C₁₈ 150 mm×40 mm×5 μm column, Mobile Phase A: H₂O (0.05% formic acid)/Mobile Phase B: ACN, 0 to 35% of Mobile Phase B over 9 min, 100% of Mobile Phase B hold 2 min, flow rate=60 mL/min) and lyophilized to provide C133 (0.200 g, 17.1% yield) as a yellow solid. (LC/MS) m/z (M+H)⁺=353.1. ¹H NMR (400 MHz, CDCl₃) δ (ppm): 8.43 (d, 1H), 8.22 (s, 1H), 8.02 (dd, 1H), 7.37 (d, 1H), 6.98 (d, 1H), 6.89 (d, 1H), 4.76 (s, 2H), 4.20-4.05 (m, 4H), 3.80 (s, 3H), 2.82 (t, 2H), 1.45 (t, 3H).

### Step 3. Preparation of N-(4-(3,3-dimethylureido)benzyl)-1-ethyl-5-(5-(1-methyl-1H-imidazol-2-yl)pyridin-2-yl)-4,5,6,7-tetrahydro-1H-pyrazolo[4,3-c]pyridine-3-carboxamide (30)

To a solution of C133 (0.080 g, 0.23 mmol) in DMSO (2 mL) was added EDCI (0.10 g, 0.54 mmol), HOPO (76 mg, 0.68 mmol) and DIEA (0.15 g, 1.1 mmol) and stirred for 30 min at 40 °C then P11 (78 mg, 0.34 mmol) was added. The reaction was stirred at 40 °C for 1 h. The residue was purified by reverse phase HPLC (C₁₈ 150 mm×30 mm×5 μm column, Mobile Phase A: H₂O (0.05% NH₄OH—NH₄HCO₃)/Mobile Phase B: ACN, 20 to 60% of Mobile Phase B over 15 min, 100% of Mobile Phase B hold 2 min, flow rate=30 mL/min) and lyophilized to provide 30 (44 mg, 37% yield) as a white solid. (LC/MS) m/z (M+H)⁺=528.4. ¹H NMR (400 MHz, CD₃OD) δ (ppm): 8.37 (d, 1H), 7.80 (dd, 1H), 7.36-7.31 (m, 2H), 7.30-7.26 (m, 2H), 7.15 (d, 1H), 7.01 (d, 1H), 6.98 (d, 1H), 4.78 (s, 2H), 4.50 (s, 2H), 4.16-4.04 (m, 4H), 3.74 (s, 3H), 3.01 (s, 6H), 2.85 (t, 2H), 1.41 (t, 3H).

### Example 34

### N-(4-(3,3-Dimethylureido)benzyl)-1-isopropyl-5-(5-(1-methyl-1H-imidazol-2-yl)pyrimidin-2-yl)-4,5,6,7-tetrahydro-1H-pyrazolo[4,3-c]pyridine-3-carboxamide (34)

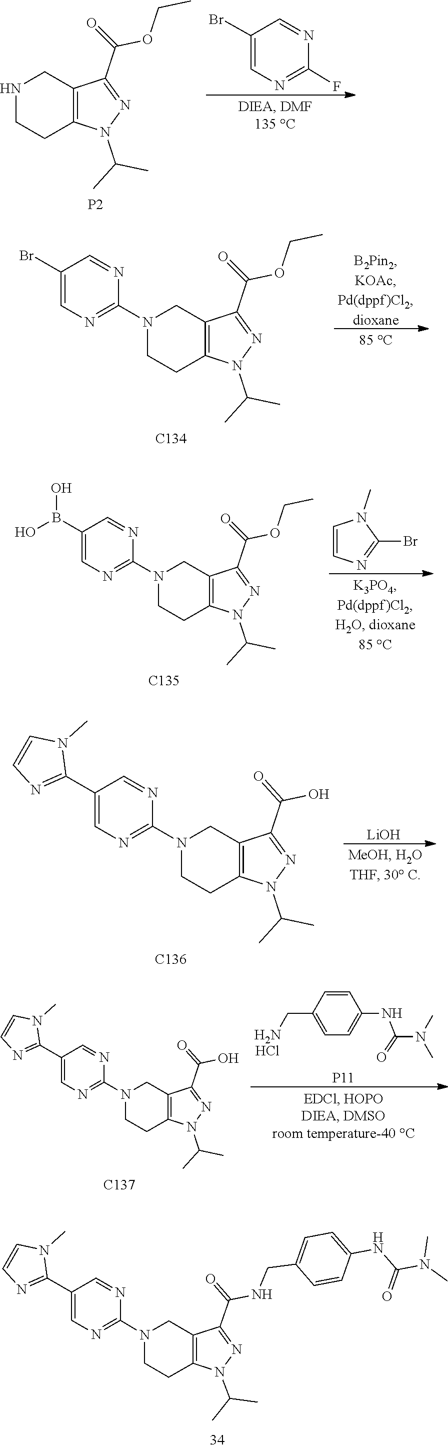

### Step 1. Preparation of ethyl 5-(5-bromopyrimidin-2-yl)-1-isopropyl-4,5,6,7-tetrahydro-1H-pyrazolo[4,3-c]pyridine-3-carboxylate (C134)

The reaction was conducted in two batches and then combined. To a solution of P2 (0.541 g, 1.98 mmol) and 5-bromo-2-fluoropyrimidine (CAS: 62802-38-4; 0.350 g, 1.98 mmol) in DMF (5 mL) was added DIEA (1.28 g, 9.89 mmol) and the reaction heated at 135 °C for 16 h to form the first batch. A second batch of the same reaction was conducted with P2 (77.4 mg, 0.283 mmol). The two batches were combined and diluted with EtOAc (50 mL). The suspension was washed with H₂O (3×30 mL) and then brine (20 mL). The organic layer was dried with Na₂SO₄ and then concentrated in vacuo. The residue was purified with column chromatography (silica gel, 0-23% EtOAc: PE) to provide C134 (0.750 g, 84.2% yield) as a white solid. (LC/MS) m/z (M+H)⁺=394.1. ¹H NMR (400 MHz, (CD₃)₂SO) δ (ppm): 8.50 (s, 2H), 4.85 (s, 2H), 4.56-4.46 (m, 1H), 4.29 (q, 2H), 4.05 (t, 2H), 2.81 (t, 2H), 1.39-1.35 (m, 6H), 1.31 (t, 3H).

### Step 2. Preparation of (2-(3-(ethoxycarbonyl)-1-isopropyl-1,4,6,7-tetrahydro-5H-pyrazolo[4,3-c]pyridin-5-yl)pyrimidin-5-yl)boronic acid (C135)

To a reaction mixture of C134 (0.350 g, 0.888 mmol) and B₂Pin₂ (0.271 g, 1.07 mmol) in dioxane (5 mL) was added AcOK (0.261 g, 2.66 mmol) and Pd(dppf)Cl₂ (52.0 mg, 0.0710 mmol). The suspension was purged with nitrogen gas for 3 min and then stirred at 85 °C for 16 h to provide C135 (0.319 g, crude) as a reaction mixture, which was used directly in the next step without further purification. (LC/MS) m/z (M+H)⁺=360.0.

### Step 3. Preparation of ethyl 1-isopropyl-5-(5-(1-methyl-1H-imidazol-2-yl)pyrimidin-2-yl)-4,5,6,7-tetrahydro-1H-pyrazolo[4,3-c]pyridine-3-carboxylate (C136)

The same reaction was conducted in two batches and then combined. To a reaction mixture of C135 (0.319 g, 0.888 mmol) and 2-bromo-1-methyl-1H-imidazole (0.214 g, 1.33 mmol) in dioxane (2.00 mL) and H₂O (0.25 mL) was added Pd(dppf)Cl₂ (65.0 mg, 0.0888 mmol) and K₃PO₄ (0.566 g, 2.66 mmol) to form the first batch. The suspension of the first batch was purged with nitrogen gas then stirred at 85 °C for 2 h.

A second batch of the same reaction was conducted with C135 (46.0 mg, 0.128 mmol). The two batches were combined and diluted with EtOAc (30 mL). The suspension was washed with H₂O (2×20 mL) and then brine (20 mL). The organic layer was dried with Na₂SO₄ and then concentrated in vacuo. The residue was purified with column chromatography (silica gel, 0-50% THF: PE) to provide C136 (0.214 g, 53.1% yield) as a brown oil. (LC/MS) m/z (M+H)⁺=396.2.

### Step 4. Preparation of 1-isopropyl-5-(5-(1-methyl-1H-imidazol-2-yl)pyrimidin-2-yl)-4,5,6,7-tetrahydro-1H-pyrazolo[4,3-c]pyridine-3-carboxylic acid (C137)

To a solution of C136 (0.214 g, 0.541 mmol) in THF (1.00 mL), H₂O (0.25 mL), and MeOH (0.25 mL) was added LiOH (68.1 mg, 1.62 mmol). The reaction mixture was stirred at 30 °C for 2 h and then extracted with EtOAc (2×10 mL). The combined organic layer was washed with H₂O (5 mL). The aqueous layer was adjusted with citric acid to pH=3 and then extracted with DCM (3×5 mL). The combined organic layer was dried with Na₂SO₄ and then concentrated in vacuo. The residue was purified by reverse phase HPLC (C₁₈ 150 mm×40 mm×5 μm column, Mobile Phase A: H₂O (NH₄OH—NH₄HCO₃)/Mobile Phase B: ACN, 8 to 48% of Mobile Phase B over 9 min, 100% of Mobile Phase B hold 2 min, flow rate=60 mL/min) and lyophilized to provide C137 (90.0 mg, 45.3% yield) as a white solid. (LC/MS) m/z (M+H)⁺=368.2.

### Step 5. Preparation of N-(4-(3,3-dimethylureido)benzyl)-1-isopropyl-5-(5-(1-methyl-1H-imidazol-2-yl)pyrimidin-2-yl)-4,5,6,7-tetrahydro-1H-pyrazolo[4,3-c]pyridine-3-carboxamide (34)

The same reaction was conducted in two batches and then combined. To a solution of C137 (0.070 g, 0.19 mmol) in DMSO (1 mL) was added HOPO (63 mg, 0.56 mmol), EDCI (88 mg, 0.46 mmol), and DIEA (0.12 g, 0.95 mmol). The reaction mixture was stirred at room temperature for 5 min, and then P11 (66 mg, 0.29 mmol) was added to form the first batch. The suspension of the first batch was stirred at 40 °C for 2 h.

A second batch of the same reaction was conducted with C137 (0.020 g, 0.054 mmol). The two batches were combined then purified with reverse phase HPLC (Boston Prime C₁₈ 150 mm×30 mm×5 μm column, Mobile Phase A: H₂O (NH₄OH—NH₄HCO₃)/Mobile Phase B: ACN, 18 to 48% of Mobile Phase B over 9 min, 100% of Mobile Phase B hold 3 min, flow rate=25 mL/min) and lyophilized to provide 34 (68 mg, 51% yield) as a white solid. (LC/MS) m/z (M+H)⁺=543.5. ¹H NMR (400 MHz, (CD₃)₂SO) δ (ppm): 8.69 (s, 2H), 8.34 (t, 1H), 8.23 (s, 1H), 7.41-7.36 (m, 2H), 7.24 (d, 1H), 7.19-7.14 (m, 2H), 6.95 (d, 1H), 4.96 (s, 2H), 4.52-4.43 (m, 1H), 4.34 (d, 2H), 4.14 (t, 2H), 3.71 (s, 3H), 2.90 (s, 6H), 2.83 (t, 2H), 1.39 (d, 6H).

### Example 35

### 5-(5-(Dimethylcarbamoyl)pyrimidin-2-yl)-N-(4-(3,3-dimethylureido)benzyl)-1-isopropyl-4,5,6,7-tetrahydro-1H-pyrazolo[4,3-c]pyridine-3-carboxamide (35)

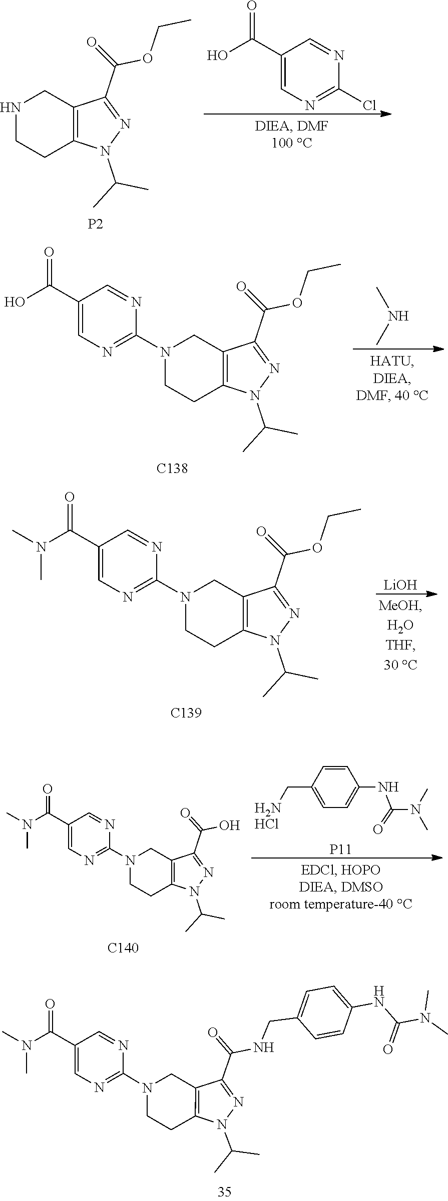

### Step 1. Preparation of 2-(3-(ethoxycarbonyl)-1-isopropyl-1,4,6,7-tetrahydro-5H-pyrazolo[4,3-c]pyridin-5-yl)pyrimidine-5-carboxylic acid (C138)

To a solution of P2 (0.350 g, 1.28 mmol) and 2-chloropyrimidine-5-carboxylic acid (CAS: 374068-01-6; 0.203 g, 1.28 mmol) in DMF (5 mL) was added DIEA (0.826 g, 6.39 mmol). The reaction mixture was stirred at 100 °C for 16 h to provide C138 (0.459 g, crude) as a suspension. The reaction mixture was used directly in the next step without further purification. (LC/MS) m/z (M+H)⁺=360.2.

### Step 2. Preparation of ethyl 5-(5-(dimethylcarbamoyl)pyrimidin-2-yl)-1-isopropyl-4,5,6,7-tetrahydro-1H-pyrazolo[4,3-c]pyridine-3-carboxylate (C139)

The same reaction was conducted in two batches and then combined. To a solution of C138 (0.459 g, 1.28 mmol) and dimethylamine hydrochloride (CAS: 506-59-2; 0.156 g, 1.91 mmol) in DMF (6 mL) was added DIEA (0.825 g, 6.39 mmol) and HATU (0.728 g, 1.92 mmol) at room temperature to form the first batch. The reaction mixture of the first batch was stirred at 40 °C for 1 h.

A second batch of the same reaction was conducted with C138 (39.0 mg, 0.108 mmol). The two batches were combined and diluted with EtOAc (50 mL). The suspension was washed with H₂O (3×30 mL) and then brine (30 mL). The organic layer was dried with Na₂SO₄ and then concentrated in vacuo. The residue was purified with column chromatography (silica gel, 0-54% THF: PE) to provide C139 (0.500 g, 89.4% yield) as an off-white solid. (LC/MS) m/z (M+H)⁺=387.3. ¹H NMR (400 MHz, (CD₃)₂SO) δ (ppm): 8.54 (s, 2H), 4.95 (s, 2H), 4.59-4.48 (m, 1H), 4.30 (q, 2H), 4.15 (t, 2H), 2.99 (br s, 6H), 2.84 (t, 2H), 1.41-1.35 (m, 6H), 1.32 (t, 3H).

### Step 3. Preparation of 5-(5-(dimethylcarbamoyl)pyrimidin-2-yl)-1-isopropyl-4,5,6,7-tetrahydro-1H-pyrazolo[4,3-c]pyridine-3-carboxylic acid (C₁₋₄₀)

The same reaction was conducted in two batches and then combined. To a solution of C139 (0.180 g, 0.466 mmol) in THF (1.00 mL), H₂O (0.25 mL), and MeOH (0.25 mL) was added LiOH (39.1 mg, 0.932 mmol) to form the first batch. The reaction mixture of the first batch was stirred at 30 °C for 2 h. A second batch of the same reaction was conducted with C139 (20.0 mg, 0.0517 mmol). The two batches were combined and extracted with EtOAc (2×10 mL). The combined organic layer was washed with H₂O (10 mL). The aqueous layer was adjusted with citric acid to pH=3 and then extracted with DCM (5×50 mL). The combined organic layer was dried with Na₂SO₄ and then concentrated in vacuo to provide C140 (0.145 g, crude) as an off-yellow solid. The solid was used directly in the next step without further purification. (LC/MS) m/z (M+H)⁺=359.3.

### Step 4. Preparation of 5-(5-(dimethylcarbamoyl)pyrimidin-2-yl)-N-(4-(3,3-dimethylureido)benzyl)-1-isopropyl-4,5,6,7-tetrahydro-1H-pyrazolo[4,3-c]pyridine-3-carboxamide (35)

The same reaction was conducted in two batches and then combined. To a solution of C140 (0.060 g, 0.17 mmol) in DMSO (1 mL) was added HOPO (55 mg, 0.49 mmol), EDCI (77 mg, 0.40 mmol), and DIEA (0.11 g, 0.84 mmol). The reaction mixture was stirred at room temperature for 5 min, and then P11 (58 mg, 0.25 mmol) was added to form the first batch. The suspension of the first batch was stirred at 40 °C for 2 h. A second batch of the same reaction was conducted with C140 (0.020 g, 0.056 mmol). The two batches were combined then purified with reverse phase HPLC (Boston Prime C₁₈ 150 mm×30 mm×5 μm column, Mobile Phase A: H₂O (NH₄OH—NH₄HCO₃)/Mobile Phase B: ACN, 15 to 45% of Mobile Phase B over 9 min, 100% of Mobile Phase B hold 3 min, flow rate=25 mL/min) and lyophilized to provide 35 (0.040 g, 38% yield) as a white solid. (LC/MS) m/z (M+H)⁺=534.5. ¹H NMR (400 MHz, (CD₃)₂SO) δ (ppm): 8.51 (s, 2H), 8.34 (t, 1H), 8.23 (s, 1H), 7.41-7.35 (m, 2H), 7.16 (d, 2H), 4.94 (s, 2H), 4.53-4.42 (m, 1H), 4.33 (d, 2H), 4.12 (t, 2H), 2.97 (br s, 6H), 2.90 (s, 6H), 2.81 (t, 2H), 1.38 (d, 6H).

### Example 36

### N-(4-(3,3-Dimethylureido)benzyl)-1-ethyl-5-(5-(2-oxopyrrolidin-1-yl)pyridin-2-yl)-4,5,6,7-tetrahydro-1H-pyrazolo[4,3-c]pyridine-3-carboxamide (36)

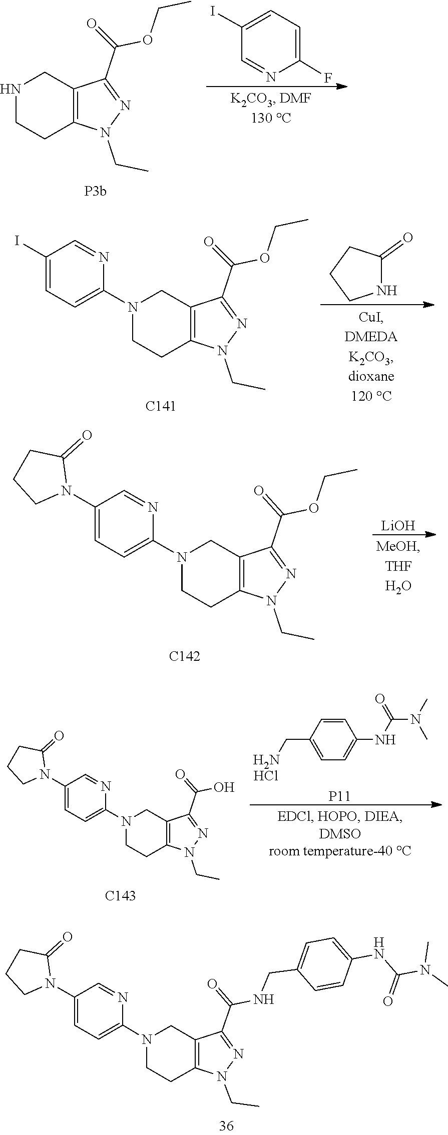

### Step 1. Preparation of ethyl 1-ethyl-5-(5-iodopyridin-2-yl)-4,5,6,7-tetrahydro-1H-pyrazolo[4,3-c]pyridine-3-carboxylate (C141)

A reaction mixture of P3b (0.803 g, 3.09 mmol), 2-fluoro-5-iodopyridine (CAS: 171197-80-1, 0.896 g, 4.02 mmol), and K₂CO₃ (1.28 g, 9.27 mmol) in DMF (20 mL) was stirred at 130 °C for 21 h. The brown reaction mixture was concentrated in vacuo and purified by column chromatography (silica gel, 0-40% EtOAc: PE) to provide C141 (0.632 g, 48.0% yield) as a white solid. (LC/MS) m/z (M+H)⁺=427.2. ¹H NMR (400 MHz, CDCl₃) δ (ppm): 8.32 (d, 1H), 7.68 (dd, 1H), 6.61-6.55 (m, 1H), 4.61 (s, 2H), 4.43 (q, 2H), 4.18-4.11 (m, 2H), 4.03 (t, 2H), 2.78 (t, 2H), 1.47-1.38 (m, 6H).

### Step 2. Preparation of ethyl 1-ethyl-5-(5-(2-oxopyrrolidin-1-yl)pyridin-2-yl)-4,5,6,7-tetrahydro-1H-pyrazolo[4,3-c]pyridine-3-carboxylate (C₁₋₄₂)

Under nitrogen gas, a reaction mixture of C141 (0.380 g, 0.891 mmol), pyrrolidine-2-one (0.114 g, 1.34 mmol), CuI (34.0 mg, 0.178 mmol), DMEDA (15.7 mg, 0.178 mmol) and K₂CO₃ (0.370 g, 2.68 mmol) in dioxane (10.0 mL) was heated at 120 °C for 16 h. The suspension was concentrated in vacuo and then purified by column chromatography (silica gel, 0-70% THF: PE) to provide C142 (0.337 g, 98.6% yield) as a white solid. (LC/MS) m/z (M+H)⁺=384.2.

### Step 3. Preparation of 1-ethyl-5-(5-(2-oxopyrrolidin-1-yl)pyridin-2-yl)-4,5,6,7-tetrahydro-1H-pyrazolo[4,3-c]pyridine-3-carboxylic acid (C143)

To a solution of C142 (0.300 g, 0.782 mmol) in THF (5.00 mL), MeOH (1.25 mL) and H₂O (2.50 mL) was added LiOH (49.7 mg, 2.07 mmol) then stirred at room temperature for 1 h. The solution was concentrated in vacuo to remove the organic solvents, then the resultant light-yellow aqueous solution was acidified to pH=3-4 with 2N HCl, which caused an off-white solid precipitate to form. The suspension was filtered, and then the filter cake was washed with H₂O (3×50 mL) and dried under high vacuo. The filter cake was collected and then lyophilized to provide C143 (0.170 g, 61.1% yield) as a white solid. ¹H NMR (400 MHz, CD₃OD) δ (ppm): 8.29 (d, 1H), 7.82 (dd, 1H), 6.91 (d, 1H), 4.67 (s, 2H), 4.14 (q, 2H), 3.97 (t, 2H), 3.89-3.81 (m, 2H), 2.84 (t, 2H), 2.60-2.52 (m, 2H), 2.23-2.13 (m, 2H), 1.40 (t, 3H).

### Step 4. Preparation of N-(4-(3,3-dimethylureido)benzyl)-1-ethyl-5-(5-(2-oxopyrrolidin-1-yl)pyridin-2-yl)-4,5,6,7-tetrahydro-1H-pyrazolo[4,3-c]pyridine-3-carboxamide (36)

A solution of C143 (0.080 mg, 0.23 mmol), HOPO (74 mg, 0.66 mmol), EDCI (0.10 g, 0.54 mmol), and DIEA (0.14 g, 1.1 mmol) in DMSO (2.5 mL) was stirred at room temperature for 10 min, then P11 (62 mg, 0.27 mmol) was added and reaction mixture was stirred at 40 °C for 2 h. The residue was purified by reverse phase HPLC (C₁₈ 150 mm×30 mm×5 μm column, Mobile Phase A: H₂O/Mobile Phase B: ACN (NH₄OH—NH₄HCO₃), 15 to 55% of Mobile Phase B over 9 min, 100% of Mobile Phase B hold 2 min, flow rate=30 mL/min) and lyophilized to provide 36 (28 mg, 23% yield) as a white solid. (LC/MS) m/z (M+H)⁺=531.5. ¹H NMR (400 MHz, (CD₃)₂SO) δ (ppm): 8.42 (t, 1H), 8.30 (d, 1H), 8.23 (s, 1H), 7.90 (dd, 1H), 7.43-7.36 (m, 2H), 7.21-7.15 (m, 2H), 6.92 (d, 1H), 4.67 (s, 2H), 4.34 (d, 2H), 4.08 (q, 2H), 3.88 (t, 2H), 3.78 (t, 2H), 2.92 (s, 6H), 2.83-2.77 (m, 2H), 2.44 (t, 2H), 2.11-2.01 (m, 2H), 1.33 (t, 3H).

### Example 37

### 5-(1-Acetyl-1,2,3,4-tetrahydroquinolin-6-yl)-N-(4-(3,3-dimethylureido)benzyl)-1-isopropyl-4,5,6,7-tetrahydro-1H-pyrazolo[4,3-c]pyridine-3-carboxamide (37)

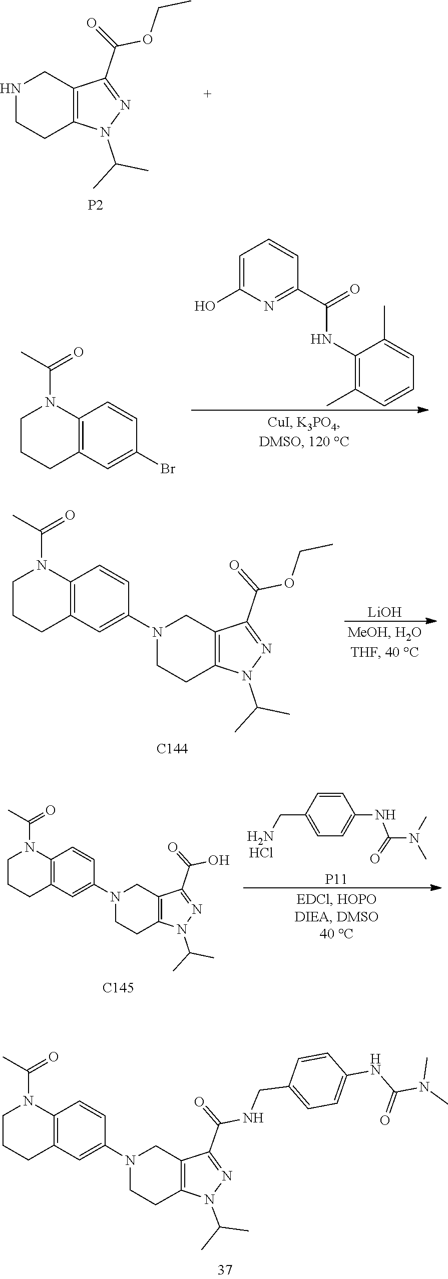

### Step 1. Preparation of ethyl 5-(1-acetyl-1,2,3,4-tetrahydroquinolin-6-yl)-1-isopropyl-4,5,6,7-tetrahydro-1H-pyrazolo[4,3-c]pyridine-3-carboxylate (C144)

The same reaction was conducted in two batches and then combined. To a solution of P2 (0.450 g, 1.90 mmol), N-(2,6-dimethylphenyl)-6-hydroxypyridine-2-carboxamide (CAS: 1982260-18-3; 0.122 g, 0.506 mmol) and 1-(6-bromo-3,4-dihydroquinolin-1(2H)-yl)ethan-1-one (CAS: 22190-40-5; 0.483 g, 1.90 mmol) in DMSO (20 mL) was added K₃PO₄ (1.07 g, 5.06 mmol) and CuI (48.2 mg, 0.253 mmol) at room temperature to form the first batch. The reaction mixture of the first batch was purged with nitrogen gas for 3 min and then stirred at 120 °C for 13 h.

A second batch of the same reaction was conducted with P2 (50.0 mg, 0.211 mmol). The two batches were combined and then concentrated in vacuo. The residue was purified with column chromatography (silica gel, 0-64% EtOAc: PE) to provide C144 (0.120 g, 15.4% yield) as a yellow solid. (LC/MS) m/z (M+H)⁺=411.1.

### Step 2. Preparation of 5-(1-acetyl-1,2,3,4-tetrahydroquinolin-6-yl)-1-isopropyl-4,5,6,7-tetrahydro-1H-pyrazolo[4,3-c]pyridine-3-carboxylic acid (C145)

To a solution C144 (0.120 g, 0.292 mmol) in THF (4 mL), MeOH (1 mL), and H₂O (2 mL), LiOH (24.5 mg, 0.585 mmol) was added at room temperature. The reaction mixture was stirred at 40 °C for 1 h and then was acidified with 2M HCl to pH=5. The acidic suspension was concentrated in vacuo and then lyophilized to provide an off-white solid. The solid was purified with reverse phase HPLC (C₁₈ 150 mm×30 mm×5 μm column, Mobile Phase A: H₂O (formic acid)/Mobile Phase B: ACN, 17 to 57% of Mobile Phase B over 15 min, 100% of Mobile Phase B hold 2 min, flow rate=30 mL/min) and lyophilized to provide C145 (70.0 mg, 62.5% yield) as a yellow solid. (LC/MS) m/z (M+H)⁺=383.3. ¹H NMR (400 MHz, CD₃OD) δ (ppm): 7.15-7.04 (m, 1H), 6.93-6.83 (m, 2H), 4.58-4.49 (m, 1H), 4.37 (s, 2H), 3.74 (t, 2H), 3.62 (t, 2H), 2.87 (t, 2H), 2.75-2.64 (m, 2H), 2.27-2.11 (m, 3H), 2.00-1.87 (m, 2H), 1.48 (d, 6H).

### Step 3. Preparation of 5-(1-acetyl-1,2,3,4-tetrahydroquinolin-6-yl)-N-(4-(3,3-dimethylureido)benzyl)-1-isopropyl-4,5,6,7-tetrahydro-1H-pyrazolo[4,3-c]pyridine-3-carboxamide (37)

To a reaction mixture of C₁₋₄₅ (0.070 g, 0.18 mmol), HOPO (61 mg, 0.55 mmol), and EDCI (84 mg, 0.44 mmol) in DMSO (2 mL) was added P11 (0.050 g, 0.22 mmol) and DIEA (0.14 g, 1.1 mmol). The suspension was stirred at room temperature for 10 min, and then the light-brown solution was stirred at 40 °C for 2 h. The light-brown reaction mixture was purified with reverse phase HPLC (C₁₈ 150 mm×30 mm×5 μm column, Mobile Phase A: H₂O (NH₄OH—NH₄HCO₃)/Mobile Phase B: ACN, 22 to 62% of Mobile Phase B over 9 min, 100% of Mobile Phase B hold 2 min) and lyophilized to provide 37 (0.055 g, 54% yield) as a white solid. (LC/MS) m/z (M+H)⁺=558.5. ¹H NMR (400 MHz, CD₃OD) δ (ppm): 7.38-7.33 (m, 2H), 7.32-7.27 (m, 2H), 7.15-7.06 (m, 1H), 6.92 (d, 2H), 4.56-4.48 (m, 3H), 4.42 (s, 2H), 3.75 (t, 2H), 3.63 (t, 2H), 3.02 (s, 6H), 2.87 (t, 2H), 2.74-2.65 (m, 2H), 2.29-2.15 (m, 3H), 2.00-1.88 (m, 2H), 1.48 (d, 6H).

### Example 39

### N-(4-(3,3-Dimethylureido)benzyl)-5-(5-((4-(3,3-dimethylureido)benzyl)carbamoyl)pyridin-2-yl)-1-isopropyl-4,5,6,7-tetrahydro-1H-pyrazolo[4,3-c]pyridine-3-carboxamide (39)

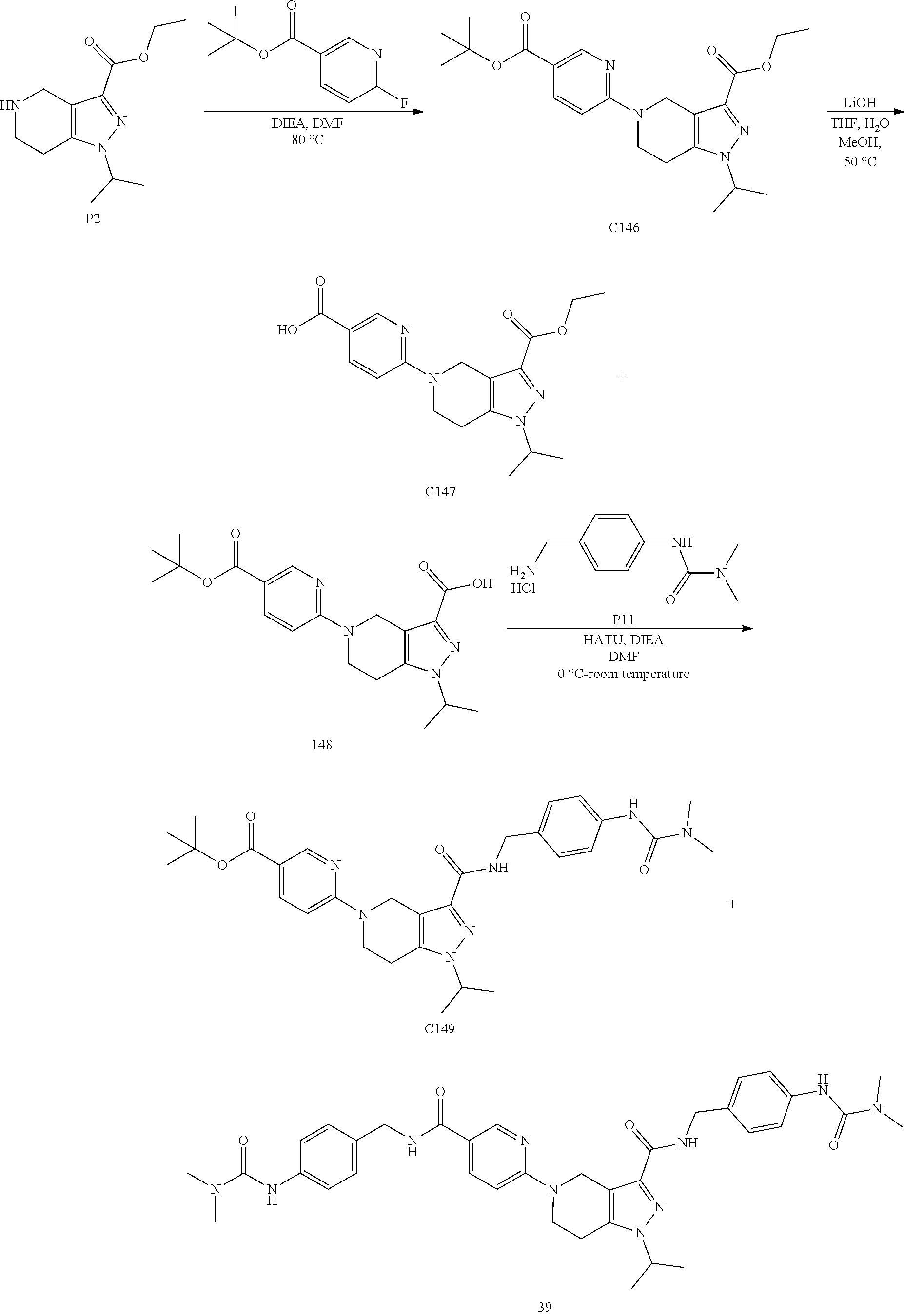

### Step 1: Preparation of ethyl 5-(5-(tert-butoxycarbonyl)pyridin-2-yl)-1-isopropyl-4,5,6,7-tetrahydro-1H-pyrazolo[4,3-c]pyridine-3-carboxylate (C146)

A reaction mixture of P2 (2.00 g, 7.30 mmol), tert-butyl 6-fluoronicotinate (CAS: 676560-01-3, 1.44 g, 7.30 mmol), and DIEA (2.83 g, 21.9 mmol) in DMF (60 mL) was stirred at 80 °C for 16 h. The reaction mixture was diluted with H₂O (200 mL) and then extracted with EtOAc (3×100 mL). The combined organic layer was washed with brine (2×100 mL) and then dried with Na₂SO₄ and filtered. The filtrate was concentrated in vacuo and then purified by column chromatography (silica gel, 0-25% EtOAc: PE) to provide C146 (1.80 g, 59.4% yield) as a white solid. ¹H NMR (400 MHz, (CD₃)₂SO) δ (ppm): 8.64 (d, 1H), 7.94 (dd, 1H), 6.94 (d, 1H), 4.79 (s, 2H), 4.59-4.47 (m, 1H), 4.31 (q, 2H), 4.08-4.00 (m, 2H), 2.85 (t, 2H), 1.53 (s, 9H), 1.39 (d, 6H), 1.34 (t, 3H).

### Step 2. Preparation of 6-(3-(ethoxycarbonyl)-1-isopropyl-1,4,6,7-tetrahydro-5H-pyrazolo[4,3-c]pyridin-5-yl)nicotinic acid (C₁₋₄₇) and 5-(5-(tert-butoxycarbonyl)pyridin-2-yl)-1-isopropyl-4,5,6,7-tetrahydro-1H-pyrazolo[4,3-c]pyridine-3-carboxylic acid (C148)

To a solution of C146 (1.80 mg, 4.34 mmol) in THF (10 mL), MeOH (5 mL) and H₂O (5 mL) was added LiOH (0.456 g, 10.9 mmol) at room temperature and then was stirred at 50 °C for 1 h. The LCMS of the reaction mixture showed 14.7% of C147 (LC/MS) m/z (M+H)⁺=331.1 and 84.6% of C148 (LC/MS) m/z (M+H)⁺=387.1 had formed. The reaction mixture was kept in the refrigerator for 48 h and then was concentrated in vacuo. The residue was diluted with H₂O (10 mL) and then acidified with 1 N HCl to pH-5, which caused a white solid precipitate. The suspension was filtered, and the filter cake was washed with H₂O (2×5 mL). The filter cake was collected then concentrated in vacuo to provide, according to LCMS, a mixture of 24% C147 and 76% C148 (1.60 g, crude) as a white solid. The solid was used directly in the next step without further purification. C147: (LC/MS) m/z (M+H)⁺=331.2. C148 (LC/MS) m/z (M+H)⁺=387.1.

### Step 5. Preparation of N-(4-(3,3-dimethylureido)benzyl)-5-(5-((4-(3,3-dimethylureido)benzyl)carbamoyl)pyridin-2-yl)-1-isopropyl-4,5,6,7-tetrahydro-1H-pyrazolo[4,3-c]pyridine-3-carboxamide (39)

At 0 °C, to a solution of C147 and C148 (0.500 g, 1.29 mmol) in DMF (10 mL) was added HATU (0.738 g, 1.94 mmol). The reaction mixture was stirred at 0 °C for 20 min then DIEA (0.669 g, 5.18 mmol) and P11 (0.446 g, 1.94 mmol) were added. After the addition, the reaction mixture was warmed to room temperature and stirred for 16 h. The LCMS of the reaction mixture showed 23.2% of 39 (LC/MS) m/z (M+H)⁺=681.4 and 43.8% of C149 (LC/MS) m/z (M+H)⁺=562.3 had formed. The suspension was diluted with H₂O (50 mL) and extracted with MeOH:DCM (1:10, 3×30 mL). The combined organic layer was washed with brine, dried with Na₂SO₄, and filtered. The filtrate was concentrated in vacuo then diluted with DMF (5 mL) and was purified by reverse phase HPLC (C18 150 mm×40 mm×5 μm column, Mobile Phase A: H₂O/Mobile Phase B: ACN (NH₄OH—NH₄HCO₃), 20 to 60% of Mobile Phase B over 9 min, 100% of Mobile Phase B hold 5 min, flow rate=30 mL/min) and lyophilized to provide 39 (108 mg, 12% yield) as a white solid. (LC/MS) m/z (M+H)⁺=681.5. ¹H NMR (400 MHz, (CD₃)₂SO) δ (ppm): 8.75 (t, 1H), 8.65 (d, 1H), 8.34 (t, 1H), 8.23 (d, 2H), 8.01 (dd, 1H), 7.41-7.35 (m, 4H), 7.20-7.12 (m, 4H), 6.90 (d, 1H), 4.76 (s, 2H), 4.54-4.42 (m, 1H), 4.35 (dd, 4H), 3.97 (t, 2H), 2.81 (t, 2H), 1.39 (d, 6H).

### Example 41

### N-(4-(3,3-Dimethylureido)benzyl)-1-isopropyl-5-(5-(5-methyl-1H-pyrazol-1-yl)pyridin-2-yl)-4,5,6,7-tetrahydro-1H-pyrazolo[4,3-c]pyridine-3-carboxamide (41)

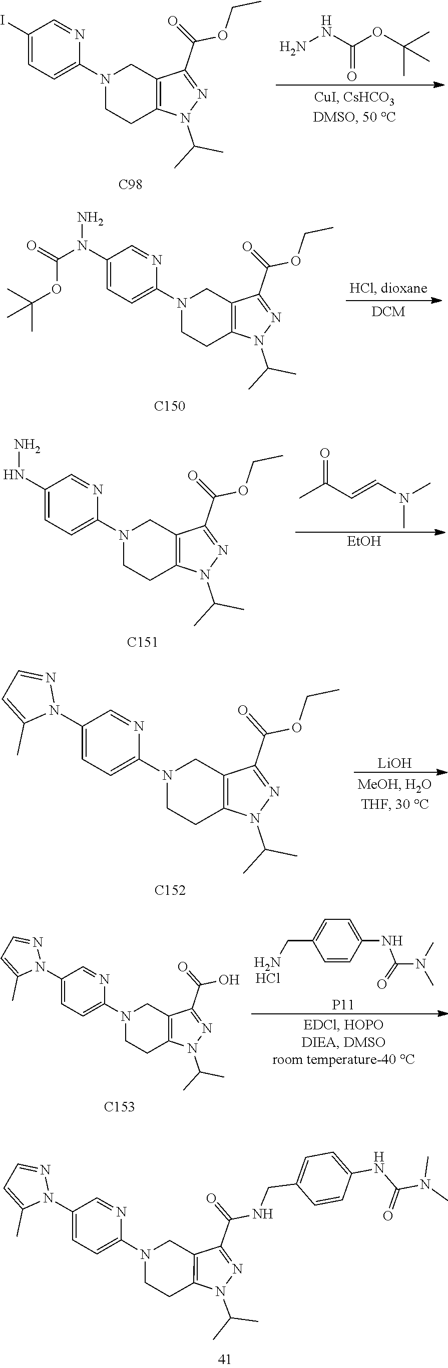

### Step 1. Preparation of ethyl 5-(5-(1-(tert-butoxycarbonyl)hydrazineyl)pyridin-2-yl)-1-isopropyl-4,5,6,7-tetrahydro-1H-pyrazolo[4,3-c]pyridine-3-carboxylate (C150)

Under nitrogen gas, to a reaction mixture of C98 (0.170 g, 0.386 mmol) in DMSO (1 mL) was added CuI (14.7 mg, 0.0772 mmol) and CsHCO₃ (0.377 g, 1.16 mmol) at room temperature. The suspension was stirred at 50 °C for 3 h and filtered through celite. The filtrate was concentrated in vacuo and then purified by column chromatography (silica gel, 0-54% THF: PE) to provide C150 (0.100 g, 58.3% yield) as a yellow oil. (LC/MS) m/z (M+H)⁺=445.2.

### Step 2. Preparation of ethyl 5-(5-hydrazineylpyridin-2-yl)-1-isopropyl-4,5,6,7-tetrahydro-1H-pyrazolo[4,3-c]pyridine-3-carboxylate hydrochloride (C151)

To a reaction mixture of C150 (0.10 g, 0.22 mmol) in DCM (1 mL) was added HCl in dioxane (8.2 mg, 0.22 mmol) and then stirred at room temperature for 16 h. The suspension was concentrated in vacuo to provide C151 (0.080 g, crude) as a solid. The solid was used directly in the next step without further purification. ¹H NMR (400 MHz, (CD₃)₂SO) δ (ppm): 10.35 (br s, 2H), 7.90 (d, 1H), 7.76 (d, 1H), 7.44-7.25 (m, 1H), 6.87 (s, 1H), 4.70 (s, 2H), 4.59-4.49 (m, 1H), 4.31 (q, 2H), 3.96 (t, 2H), 2.95-2.87 (m, 2H), 1.40-1.36 (m, 6H), 1.35-1.30 (m, 3H).

### Step 3. Preparation of ethyl 1-isopropyl-5-(5-(5-methyl-1H-pyrazol-1-yl)pyridin-2-yl)-4,5,6,7-tetrahydro-1H-pyrazolo[4,3-c]pyridine-3-carboxylate (C152)

To a reaction mixture of C151 (0.080 g, 0.21 mmol) in EtOH (1 mL) was added 4-(dimethylamino)but-3-en-2-one (CAS: 1190-91-6, 24 mg, 0.21 mmol) and then stirred at room temperature for 16 h. The suspension was filtered through celite, and the filtrate was concentrated in vacuo. The residue was purified by column chromatography (silica gel, 0-30% EtOAc: PE) to provide C152 (28 mg, 31% yield) as a yellow oil. ¹H NMR (400 MHz, CDCl₃) δ (ppm): 8.24 (d, 1H), 7.63-7.57 (m, 1H), 7.56 (d, 1H), 6.81 (d, 1H), 6.18 (s, 1H), 4.70 (s, 2H), 4.55-4.36 (m, 3H), 4.16-4.12 (m, 2H), 2.86 (t, 2H), 2.30 (s, 3H), 1.53 (d, 6H), 1.43 (t, 3H).

### Step 4. Preparation 1-isopropyl-5-(5-(5-methyl-1H-pyrazol-1-yl)pyridin-2-yl)-4,5,6,7-tetrahydro-1H-pyrazolo[4,3-c]pyridine-3-carboxylic acid (C153)

To a solution of C152 (25 mg, 0.063 mmol) in THF (1.00 mL), H₂O (0.25 mL), and MeOH (0.25 mL) was added LiOH (5.3 mg, 0.13 mmol). The reaction mixture was stirred at 30 °C for 2 h. The suspension's pH was adjusted with citric acid to pH=3 and concentrated in vacuo to provide C153 (23 mg, crude) as a yellow solid. The solid was used directly in the next step without further purification.

### Step 5. Preparation N-(4-(3,3-dimethylureido)benzyl)-1-isopropyl-5-(5-(5-methyl-1H-pyrazol-1-yl)pyridin-2-yl)-4,5,6,7-tetrahydro-1H-pyrazolo[4,3-c]pyridine-3-carboxamide (41)

To a solution of C153 (25 mg, 0.068 mmol) in DMSO (1 mL) was added HOPO (22 mg, 0.20 mmol), EDCI (31 mg, 0.16 mmol), and DIEA (44 mg, 0.34 mmol). The suspension was stirred at room temperature for 5 min, and then P11 (23 mg, 0.10 mmol) was added. The reaction mixture was stirred at 40 °C for 2 h then purified with reverse phase HPLC (Boston Prime C₁₈ 150 mm×30 mm×5 μm column, Mobile Phase A: H₂O (NH₄OH—NH₄HCO₃)/Mobile Phase B: ACN, 24 to 54% of Mobile Phase B over 9 min, 100% of Mobile Phase B hold 3 min, flow rate=50 mL/min) and lyophilized to provide 41 (26 mg, 71% yield) as a white solid. (LC/MS) m/z (M+H)⁺=542.4. ¹H NMR (400 MHz, CDCl₃) δ (ppm): 8.22 (d, 1H), 7.58-7.53 (m, 2H), 7.38-7.33 (m, 2H), 7.30 (d, 2H), 7.15 (t, 1H), 6.86 (d, 1H), 6.31 (s, 1H), 6.17 (s, 1H), 4.76 (s, 2H), 4.56 (d, 2H), 4.36-4.31 (m, 1H), 4.15 (t, 2H), 3.03 (s, 6H), 2.82 (t, 2H), 2.29 (s, 3H), 1.45 (d, 6H).

### Example 42

### N-(4-(3,3-Dimethylureido)benzyl)-7-(5-(1-methyl-1H-imidazol-2-yl)pyridin-2-yl)-3-(trifluoromethyl)-5,6,7,8-tetrahydroimidazo[1,5-a]pyrazine-1-carboxamide (42)

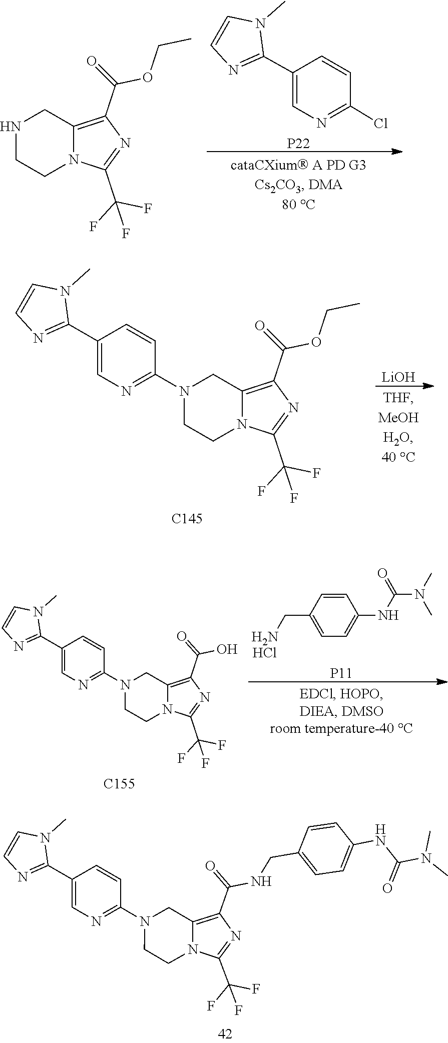

### Step 1. Preparation of ethyl 7-(5-(1-methyl-1H-imidazol-2-yl)pyridin-2-yl)-3-(trifluoromethyl)-5,6,7,8-tetrahydroimidazo[1,5-a]pyrazine-1-carboxylate (C154)

To the reaction mixture of ethyl 5,6,7,8-tetrahydro-3-(trifluoromethyl)imidazo[1,5-a]pyrazine-1-carboxylate (CAS: 1598419-92-1; 0.300 g, 1.14 mmol) and P22 (0.243 g, 1.25 mmol) in DMA (10 mL) was added cataCXium™ A Pd G3 (83.0 mg, 0.114 mmol) and Cs₂CO₃ (1.11 g, 3.42 mmol). The suspension was degassed with nitrogen gas and then stirred at 80 °C for 16 h. The reaction mixture was diluted with DCM (3×50 mL). The combined organic layer was washed with H₂O (50 mL), then brine (50 mL), and dried with Na₂SO₄. The suspension was concentrated in vacuo and then purified by column chromatography (silica gel, 4% MeOH: EtOAc) to provide C154 (0.258 g, 53.8% yield) as a yellow gum. (LC/MS) m/z (M+H)⁺=421.2.

### Step 2. Preparation of 7-(5-(1-methyl-1H-imidazol-2-yl)pyridin-2-yl)-3-(trifluoromethyl)-5,6,7,8-tetrahydroimidazo[1,5-a]pyrazine-1-carboxylic acid (C155)

To C154 (0.258 g, 0.614 mmol) in THF (4.0 mL), H₂O (2.0 mL), and MeOH (1.0 mL) was added LiOH (44.1 mg, 1.84 mmol) at room temperature. The reaction mixture was stirred at 40 °C. for 1.5 h and then was concentrated in vacuo to remove the organic layer. The resultant aqueous layer was acidified to pH=5-6 with 2N HCl, which caused an off-white solid precipitate. The suspension was filtered, and then the filter cake was washed with H₂O (3×10 mL) and dried under high vacuo. The filter cake was collected then lyophilized to provide C155 (0.120 g, 49.8% yield) as a yellow solid. ¹H NMR (400 MHz, (CD₃)₂SO) δ (ppm): 12.48 (br s, 1H), 8.51 (d, 1H), 7.95 (dd, 1H), 7.24 (s, 1H), 7.10 (d, 1H), 6.97 (s, 1H), 5.12 (s, 2H), 4.31 (t, 2H), 4.14 (t, 2H), 3.73 (s, 3H)

### Step 3. Preparation of N-(4-(3,3-dimethylureido)benzyl)-7-(5-(1-methyl-1H-imidazol-2-yl)pyridin-2-yl)-3-(trifluoromethyl)-5,6,7,8-tetrahydroimidazo[1,5-a]pyrazine-1-carboxamide (42)

To a solution of C155 (0.050 g, 0.13 mmol) in DMSO (2.0 mL) was added HOPO (42 mg, 0.38 mmol), DIEA (82 mg, 0.64 mmol), and EDCI (59 mg, 0.31 mmol). The reaction mixture was stirred for 10 min at room temperature, and then P11 (38 mg, 0.17 mmol) was added. The reaction mixture was stirred at 40 °C for 3 h then purified by reverse phase HPLC (Phenomenex Gemini NX C₁₈ 150 mm×30 mm×5 μm column, Mobile Phase A: H₂O (NH₄OH—NH₄HCO₃)/Mobile Phase B: ACN, 22 to 62% of Mobile Phase B over 9 min, 100% of Mobile Phase B hold 2 min, flow rate=60 mL/min) and lyophilized to provide 42 (0.030 mg, 42% yield) as a white solid. (LC/MS) m/z (M+H)⁺=568.3. ¹H NMR (400 MHz, CD₃OD) δ (ppm): 8.43 (d, 1H), 7.86 (dd, 1H), 7.35-7.30 (m, 2H), 7.29-7.24 (m, 2H), 7.16 (d, 1H), 7.07-7.00 (m, 2H), 5.15 (s, 2H), 4.49 (s, 2H), 4.34 (t, 2H), 4.21 (t, 2H), 3.74 (s, 3H), 3.00 (s, 6H).

### Example 43

### 5-(5-(Dimethylcarbamoyl)pyridin-2-yl)-1-isopropyl-N-(4-(6-oxo-1,6-dihydropyrimidin-2-yl)benzyl)-4,5,6,7-tetrahydro-1H-pyrazolo[4,3-c]pyridine-3-carboxamide (43)

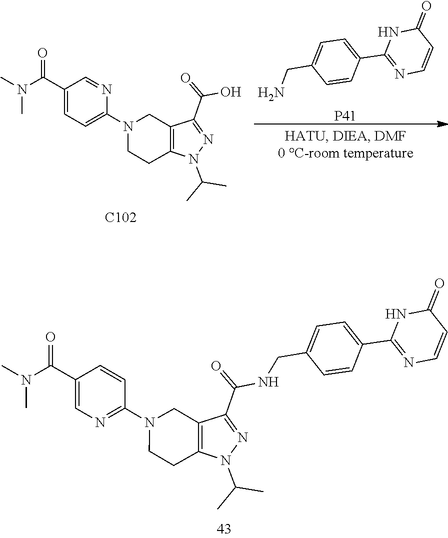 [SMILES: "C102" ; CC(C)n1nc(C(=O)O)c2c1CCN(c1ccc(C(=O)N(C)C)cn1)C2 ; "P41" ; NCc1ccc(-c2nccc(=O)[nH]2)cc1 ; CC(C)n1nc(C(=O)NCc2ccc(-c3nccc(=O)[nH]3)cc2)c2c1CCN(c1ccc(C(=O)N(C)C)cn1)C2]

### Step 1. Preparation of 5-(5-(dimethylcarbamoyl)pyridin-2-yl)-1-isopropyl-N-(4-(6-oxo-1,6-dihydropyrimidin-2-yl)benzyl)-4,5,6,7-tetrahydro-1H-pyrazolo[4,3-c]pyridine-3-carboxamide (43)

Under nitrogen gas at 0 °C, to a solution of C102 (91 mg, 0.38 mmol), P41 (0.14 g, 0.38 mmol) and HATU (0.15 g, 0.40 mmol) in DMF (1.9 mL) was added DIEA (0.22 g, 1.7 mmol). The reaction mixture was stirred at room temperature for 2 h then purified by reverse phase HPLC (Boston Prime C₁₈ 150 mm×30 mm×5 μm column, Mobile Phase A: H₂O (0.05% formic acid)/Mobile Phase B: ACN, 15 to 35% of Mobile Phase B over 11.0 min, 95% of Mobile Phase B hold 2.0 N-(4-(3,3-Dimethylureido)benzyl)-1-isopropyl-5-(5-(1-methyl-1H-imidazol-2-yl)pyridin-2-yl)-4,5,6,7-tetrahydro-1H-pyrazolo[4,3-c]pyridine-3-carboxamide (45) min, flow rate=35 mL/min) to provide 43 (0.010 g, 4.8% yield) as a white solid. (LC/MS) m/z (M+H)⁺=541.3. ¹H NMR (400 MHz, (CD₃)₂SO) δ (ppm): 8.57 (t, 1H), 8.23 (d, 1H), 8.09-8.02 (m, 3H), 7.63 (dd, 1H), 7.43 (d, 2H), 6.88 (d, 1H), 6.29 (d, 1H), 4.74 (s, 2H), 4.57-4.41 (m, 3H), 3.95 (t, 2H), 2.96 (s, 6H), 2.82 (t, 2H), 1.40 (d, 6H).

### Example 45

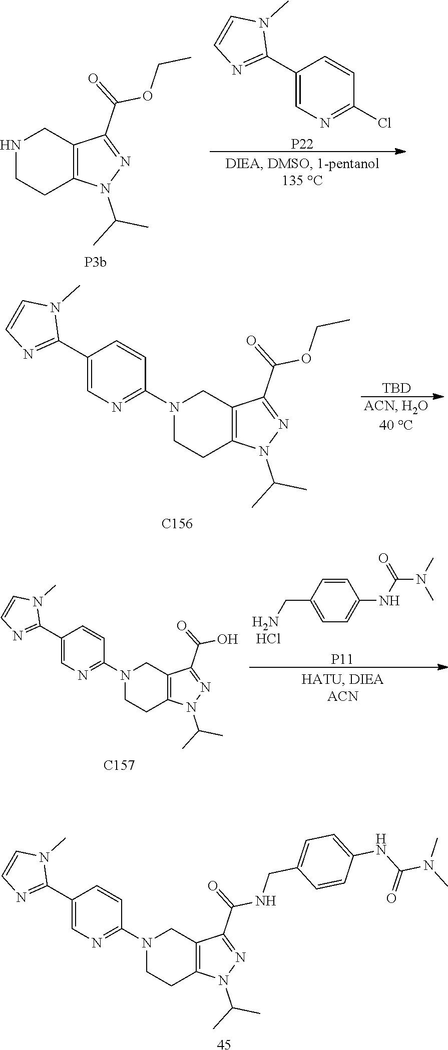

### Step 1. Preparation of ethyl 1-isopropyl-5-(5-(1-methyl-1H-imidazol-2-yl)pyridin-2-yl)-4,5,6,7-tetrahydro-1H-pyrazolo[4,3-c]pyridine-3-carboxylate (C156)

A reaction mixture of P3b (0.100 g, 0.421 mmol), P22 (97.9 mg, 0.506 mmol), and DIEA (0.218 g, 1.69 mmol) in DMSO (1 mL) and 1-pentanol (1 mL) was heated at 135 °C for 17 h. The solution was diluted with DCM and brine and then concentrated in vacuo. The residue was redissolved in DCM and then purified by column chromatography (silica gel, 0-10% MeOH: DCM) to provide C156 (0.130 g, 78.3% yield) as a yellow oil. (LC/MS) m/z (M+H)⁺=395.3.

### Step 2. Preparation of 1-isopropyl-5-(5-(1-methyl-1H-imidazol-2-yl)pyridin-2-yl)-4,5,6,7-tetrahydro-1H-pyrazolo[4,3-c]pyridine-3-carboxylic acid (C157)

To a solution of C156 (0.050 g, 0.13 mmol) in ACN (1 mL) and H₂O (46 μL) was added TBD (53 mg, 0.38 mmol). The reaction mixture was stirred at 40 °C for 1 h and 20 min, and then NaOH (0.020 g, 0.51 mmol) was added. The suspension was stirred at room temperature for 15 min, and then 12.1M HCl (18 mg, 0.51 mmol) was added. The reaction mixture was concentrated in vacuo to provide C157 (0.046 g, crude) as a yellow oil. The oil was used directly in the next step without further purification.

Step 3. Preparation of N-(4-(3,3-dimethylureido)benzyl)-1-isopropyl-5-(5-(1-methyl-1H-imidazol-2-yl)pyridin-2-yl)-4,5,6,7-tetrahydro-1H-pyrazolo[4,3-c]pyridine-3-carboxamide (45)

A reaction mixture of C157 (46 mg, 0.13 mmol), P11 (35 mg, 0.15 mmol), DIEA (49 mg, 0.38 mmol), and HATU (48 mg, 0.13 mmol) in ACN (1 mL) was stirred at room temperature for 21 h and then concentrated in vacuo. The residue was dissolved in DMSO then acidified with TFA and purified by reverse phase HPLC (Sunfire C₁₈ 100 mm×19 mm×5 μm column, Mobile Phase A: H₂O/Mobile Phase B: ACN (0.05% TFA), 5 to 95% of Mobile Phase B over 9 min, 95% of Mobile Phase B hold 1 min, flow rate=25 mL/min) and lyophilized to provide 45 (4.3 mg, 6.3% yield). (LC/MS) m/z (M+H)⁺=542.3. ¹H NMR (600 MHz, (CD₃)₂SO) δ (ppm): 8.44 (d, 1H), 8.34 (t, 1H), 8.23 (s, 1H), 7.86 (dd, 1H), 7.42-7.37 (m, 2H), 7.24 (br s, 1H), 7.20-7.16 (m, 2H), 6.98 (d, 2H), 4.77 (s, 2H), 4.55-4.47 (m, 1H), 4.35 (d, 2H), 3.97 (t, 2H), 3.72 (s, 3H) 2.91 (s, 6H), 2.85 (t, 2H), 1.41 (d, 6H).

### Example 77

### rac-(R)-5-(5-(Dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (77)

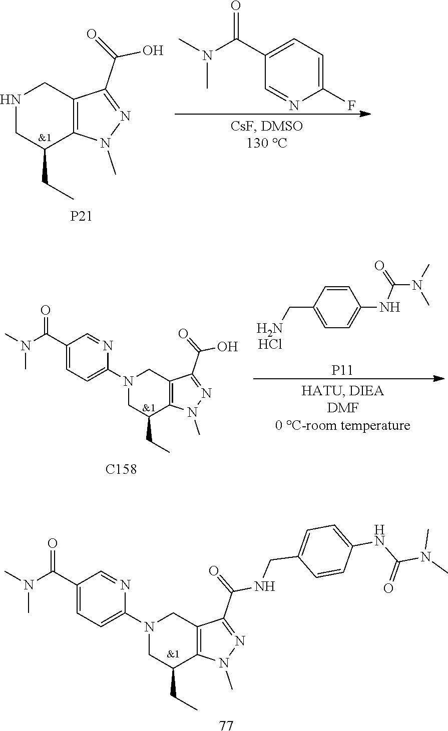

### Step 1. Preparation of rac-(R)-5-(5-(dimethylcarbamoyl)pyridin-2-yl)-7-ethyl-1-methyl-4,5,6,7-tetrahydro-1H-pyrazolo[4,3-c]pyridine-3-carboxylic acid (C158)

To a reaction mixture of P21 (0.186 g, 0.757 mmol) and CsF (0.287 g, 1.89 mmol) in DMSO (3 mL) was added 6-fluoro-N,N-dimethylnicotinamide (CAS: 1032251-82-3; 0.127 g, 0.757 mmol). The suspension was stirred at 130 °C for 8 h and then was diluted with H₂O (20 mL). The reaction mixture was cooled to 0 °C, and then aqueous Na₂CO₃ was added to pH=11 and extracted with EtOAc (2×25 mL). The aqueous layer was collected and adjusted to pH=3 with 1 M HCl. The acidic aqueous phase was extracted with (1:10, MeOH: DCM, 3×20 mL). The combined organic layer was washed with brine (2×25 mL), dried over Na₂SO₄, filtered, and concentrated in vacuo to provide C158 (0.271 g, crude) as a yellow oil. The oil was used directly in the next step without further purification. ¹H NMR (400 MHz, (CD₃)₂SO) δ (ppm): 12.67 (s, 1H), 8.22 (d, 1H), 7.61 (dd, 1H), 6.82 (d, 1H), 4.97 (d, 1H), 4.64 (d, 1H), 4.16 (d, 1H), 3.83-3.74 (m, 3H), 3.19-3.12 (m, 1H), 3.00-2.90 (m, 7H), 1.62-1.51 (m, 1H), 1.46-1.33 (m, 1H), 0.99 (t, 3H).

### Step 2. Preparation of rac-(R)-5-(5-(dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (77)

At 0 °C, to a solution of C158 (0.27 g, 0.68 mmol) in DMF (5 mL) was added HATU (0.28 g, 0.75 mmol). The reaction mixture was stirred at 0 °C for 20 min, and then DIEA (0.35 g, 2.7 mmol) and P11 (0.16 g, 0.68 mmol) were added at 0 °C. The suspension was stirred at room temperature for 2 h then diluted with H₂O (50 mL) and extracted with (1:10, MeOH: DCM, 3×40 mL). The combined organic layer was dried with Na₂SO₄, filtered, and concentrated in vacuo to give a yellow gum. The residue was purified by column chromatography (silica gel; 0-10% MeOH: DCM) to provide 77 (0.32 g, 88% yield) as a yellow solid. (LC/MS) m/z (M+H)⁺=533.4. ¹H NMR (400 MHz, (CD₃)₂SO) b (ppm): 8.51 (t, 1H), 8.27-8.20 (m, 2H), 7.65 (dd, 1H), 7.41-7.35 (m, 2H), 7.18-7.12 (m, 2H), 6.83 (d, 1H), 5.01 (d, 1H), 4.73 (d, 1H), 4.40-4.18 (m, 3H), 3.82 (s, 3H), 3.23-3.09 (m, 1H), 3.04-2.94 (m, 7H), 2.93-2.87 (m, 6H), 1.66-1.53 (m, 1H), 1.49-1.36 (m, 1H), 1.03 (t, 3H).

### Example 58 and 78

### rel-(R or S)-5-(5-(Dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (58 and 78)

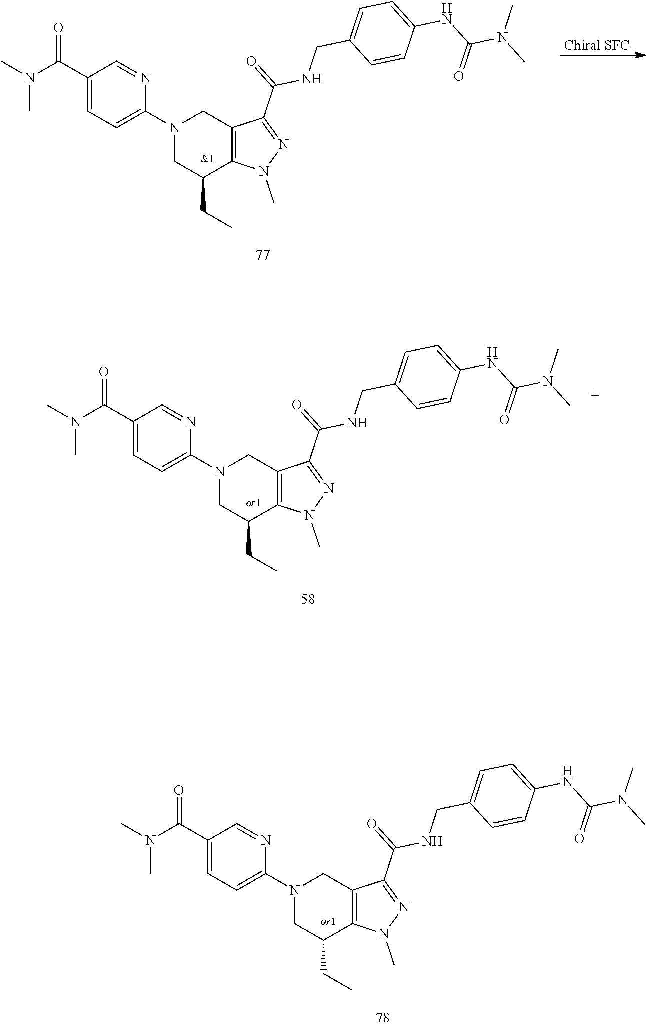

### Step 1. Preparation of rel-(R or S)-5-(5-(dimethylcarbamoyl)pyridin-2-yl)-N-(4-(3,3-dimethylureido)benzyl)-7-ethyl-1-methyl-4,5,6,7-tetrahydro-1H-pyrazolo[4,3-c]pyridine-3-carboxamide (58 and 78)

Example 77 (0.29 g, 0.81 mmol) was separated using chiral SFC [Column: DAICEL CHIRALCEL OD 250 mm×30 mm×10 μm; Mobile phase: 40% CO₂/60% MeOH (0.1% NH₄OH); flow rate: 80 mL/min; gradient time=45 min] to provide 58 (87 mg, 30% yield) as a white solid and 78 (83 mg, 29% yield) as a white solid. The chiral purity was determined using SFC analytical method on Chiralcel OD-3 50 mm×4.6 mm×3 μm column; Mobile phase A: CO₂; Mobile phase B: MeOH with 0.2% of NH₃; isocratic: 40% of Mobile Phase B; flow rate: 3.5 mL/min; backpressure: 1500 psi; column temperature: 35 °C to provide Example 58: peak 1 ((SFC-MS) m/z (M+H)⁺=533.41 at retention time: 0.778 min, 100% ee). ¹H NMR (400 MHz, (CD₃)₂SO) δ (ppm): 8.52 (t, 1H), 8.27-8.21 (m, 2H), 7.65 (dd, 1H), 7.40-7.34 (m, 2H), 7.18-7.11 (m, 2H), 6.83 (d, 1H), 5.00 (d, 1H), 4.76-4.69 (m, 1H), 4.42-4.16 (m, 3H), 3.81 (s, 3H), 3.24-3.15 (m, 1H), 3.02-2.93 (m, 7H), 2.92-2.87 (m, 6H), 1.66-1.53 (m, 1H), 1.50-1.36 (m, 1H), 1.02 (t, 3H) and Example 78: peak 2 ((SFC-MS) m/z (M+H)⁺=533.41 at retention time: 1.392 min, 100% ee). ¹H NMR (400 MHz, (CD₃)₂SO) δ (ppm): 8.49 (t, 1H), 8.24-8.16 (m, 2H), 7.61 (dd, 1H), 7.36-7.31 (m, 2H), 7.16-7.09 (m, 2H), 6.80 (d, 1H), 4.97 (d, 1H), 4.69 (d, 1H), 4.35-4.15 (m, 3H), 3.78 (s, 3H), 3.20-3.10 (m, 1H), 2.98-2.91 (m, 7H), 2.87 (m, 6H), 1.62-1.49 (m, 1H), 1.46-1.32 (m, 1H), 0.99 (t, 3H).

### Example 102

### N-(4-Aminobenzyl)-5-(5-(dimethylcarbamoyl)pyridin-2-yl)-1-isopropyl-4,5,6,7-tetrahydro-1H-pyrazolo[4,3-c]pyridine-3-carboxamide (102)

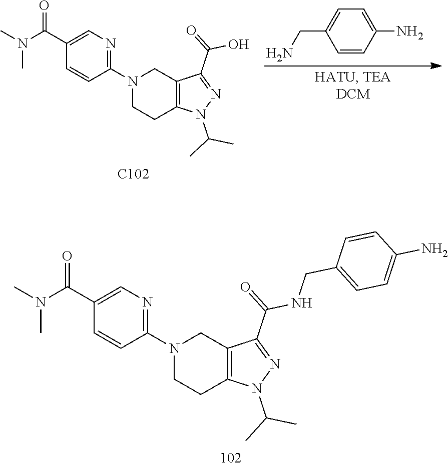 [SMILES: CC(C)n1nc(C(=O)O)c2c1CCN(c1ccc(C(=O)N(C)C)cn1)C2 ; NCc1ccc(N)cc1 ; "C102" ; CC(C)n1nc(C(=O)NCc2ccc(N)cc2)c2c1CCN(c1ccc(C(=O)N(C)C)cn1)C2]

### Step 1. Preparation of N-(4-aminobenzyl)-5-(5-(dimethylcarbamoyl)pyridin-2-yl)-1-isopropyl-4,5,6,7-tetrahydro-1H-pyrazolo[4,3-c]pyridine-3-carboxamide (102)

To a reaction mixture of C102 (1.00 g, 2.80 mmol) in DCM (15 mL) were added 4-aminobenzylamine (CAS: 4403-71-8; 0.431 g, 3.53 mmol), HATU (2.13 g, 5.60 mmol), and TEA (0.581 g, 5.74 mmol). The suspension was stirred at room temperature for 2 h, then diluted with DCM and washed with H₂O. The organic layer was dried with Na₂SO₄, filtered, and concentrated in vacuo. The residue was purified by column chromatography (silica gel, 0-10% MeOH: DCM) to provide 102 (1.58 g, >95.0% yield) as a yellow foam. (LCMS) m/z (M+H)⁺=462.6. ¹H NMR (500 MHz, (CD₃)₂SO) δ (ppm): 8.24 (d, 1H), 8.11 (t, 1H), 7.64 (dd, 1H), 6.99 (d, 2H), 6.88 (d, 1H), 6.50 (d, 2H), 4.94 (s, 2H), 4.74 (s, 2H), 4.54-4.43 (m, 1H), 4.24 (d, 2H), 3.95 (t, 2H), 2.97 (s, 6H), 2.81 (t, 2H), 1.38 (d, 6H).

### Example 113

### (R)-5-(5-(Dimethylcarbamoyl)pyridin-2-yl)-N-(4-(3,3-dimethylureido)benzyl)-1-ethyl-6-methyl-4,5,6,7-tetrahydro-1H-pyrazolo[4,3-c]pyridine-3-carboxamide (113)

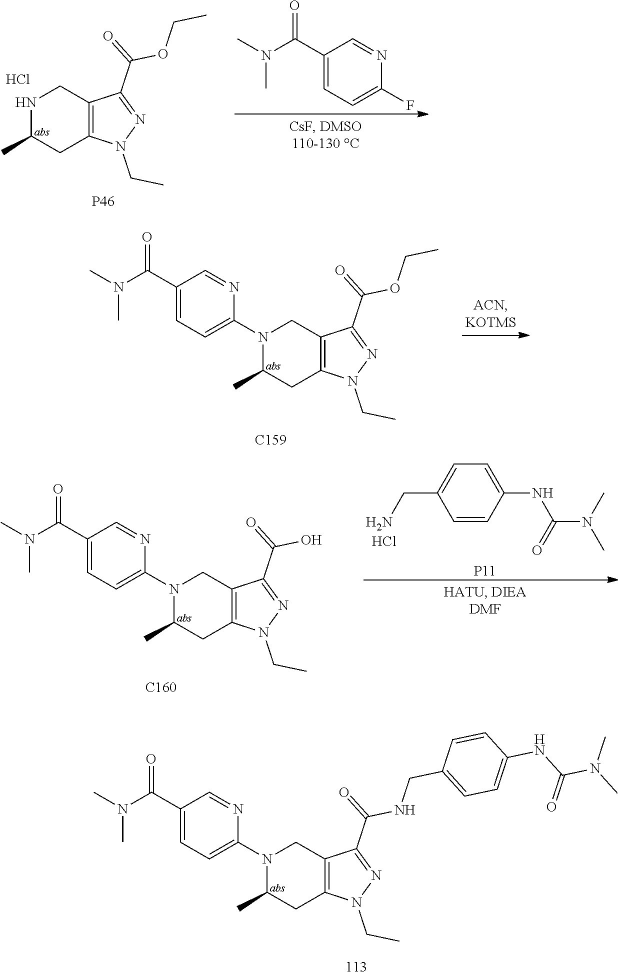

### Step 1. Preparation of ethyl (R)-5-(5-(dimethylcarbamoyl)pyridin-2-yl)-1-ethyl-6-methyl-4,5,6,7-tetrahydro-1H-pyrazolo[4,3-c]pyridine-3-carboxylate (C159)

To a solution of P46 (0.380 g, 1.39 mmol) and 6-fluoro-N,N-dimethylnicotinamide (CAS: 1032251-82-3; 233 mg, 1.39 mmol) in DMSO (5 mL) was added CsF (527 mg, 3.47 mmol). The reaction mixture was heated at 130 °C for 8 h, and then an additional portion of 6-fluoro-N,N-dimethylnicotinamide (CAS: 1032251-82-3; 233 mg, 1.39 mmol) was added. The suspension was heated at 110 °C for 24 h, and then an additional portion of CsF (274 mg, 1.80 mmol) was added. The reaction mixture was stirred at 130 °C for 16 h and then was diluted with H₂O (10 mL). The suspension was extracted with EtOAc (3×10 mL). The combined organic layer was washed with brine (2×10 mL), dried over Na₂SO₄, filtered, and concentrated in vacuo. The residue was purified by column chromatography (silica gel, 0-100% EtOAc: PE) to provide C159 (0.250 g, 46.7% yield) as a yellow gum. (LC/MS) m/z (M+H)⁺=386.2. ¹H NMR (600 MHz, (CD₃)₂SO) δ (ppm): 8.30-8.27 (m, 1H), 7.68 (dd, 1H), 6.89 (d, 1H), 5.33-5.28 (m, 1H), 5.14 (d, 1H), 4.31 (q, 2H), 4.18-4.09 (m, 3H), 3.04-2.93 (m, 7H), 2.77 (d, 1H), 1.36-1.30 (m, 6H), 1.08 (d, 3H).

### Step 2. Preparation of (R)-5-(5-(dimethylcarbamoyl)pyridin-2-yl)-1-ethyl-6-methyl-4,5,6,7-tetrahydro-1H-pyrazolo[4,3-c]pyridine-3-carboxylic acid (C160)

To a reaction mixture of C159 (0.250 g, 0.649 mmol) in ACN (5 mL) was added KOTMS (166 mg, 1.30 mmol) at room temperature under nitrogen gas and then stirred for 1 h. The solution was acidified with HCl in dioxane (47.3 mg, 1.30 mmol) to a pH of 5-6, then concentrated in vacuo to provide C160 (232 mg, crude) as a yellow gum. The gum was used directly in the next step without further purification. (LC/MS) m/z (M+H)⁺=358.1. ¹H NMR (600 MHz, (CD₃)₂SO) δ (ppm): 8.26 (d, 1H), 7.71 (d, 1H), 6.95 (d, 1H), 5.25-5.20 (m, 1H), 5.12 (d, 1H), 4.17-4.06 (m, 3H), 2.98 (s, 7H), 2.77 (d, 1H), 1.32 (t, 3H), 1.08 (d, 3H).

### Step 3. Preparation of (R)-5-(5-(dimethylcarbamoyl)pyridin-2-yl)-N-(4-(3,3-dimethylureido)benzyl)-1-ethyl-6-methyl-4,5,6,7-tetrahydro-1H-pyrazolo[4,3-c]pyridine-3-carboxamide (113)

To a reaction mixture of C160 (232 mg, 0.649 mmol) and P11 (125 mg, 0.649 mmol) in DMF (2.5 mL) were added HATU (271 mg, 0.714 mmol) and DIEA (336 mg, 2.60 mmol). The suspension was stirred at room temperature for 16 h, diluted with H₂O (15 mL), and extracted with EtOAc (3×15 mL). The combined organic layer was washed with H₂O (2×10 mL), 1 M HCl (2×10 mL), 1 M Na₂CO₃ (2×10 mL), and then brine (1×). The organic layer was dried with Na₂SO₄, filtered, and concentrated in vacuo to provide a yellow solid. The solid was purified by reverse phase HPLC (Boston Prime C₁₈ 150 mm×30 mm×5 μm column, Mobile Phase A: H₂O (NH₄OH—NH₄HCO₃)/Mobile Phase B: ACN, 18 to 58% of Mobile Phase B over 10.0 min, 100% of Mobile Phase B hold 2.5 min, flow rate=30 mL/min) then lyophilized to provide 113 (134 mg, 38.8% yield) as a white solid. (LC/MS) m/z (M+H)⁺=533.3. ¹H NMR (400 MHz, (CD₃)₂SO) δ (ppm): 8.47 (t, 1H), 8.26 (d, 1H), 8.23 (s, 1H), 7.65 (dd, 1H), 7.41-7.34 (m, 2H), 7.21-7.15 (m, 2H), 6.85 (d, 1H), 5.31-5.22 (m, 1H), 5.14 (d, 1H), 4.33 (d, 2H), 4.17-4.01 (m, 3H), 3.01-2.93 (m, 7H), 2.90 (s, 6H), 2.77-2.69 (m, 1H), 1.32 (t, 3H), 1.06 (d, 3H). The chiral purity was determined using SFC analytical method on Chiralpak AD-3 50 mm×4.6 mm×3 μm column; Mobile phase A: CO₂/Mobile phase B: EtOH with 0.2% of NH₃; isocratic gradient: 40% of Mobile Phase B; backpressure: 1500 psi; flow rate: 3.5 mL/min; column temperature: 35 °C to provide 113: peak 1 ((SFC-MS) m/z (M+H)⁺=533.43 at retention time: 1.503 min, 100% ee).

### Example 125

(S)-5-(5-(Dimethylcarbamoyl)pyrimidin-2-yl)-N-(4-(3,3-dimethylureido)benzyl)-1-ethyl-7-methyl-4,5,6,7-tetrahydro-1H-pyrazolo[4,3-c]pyridine-3-carboxamide (125)

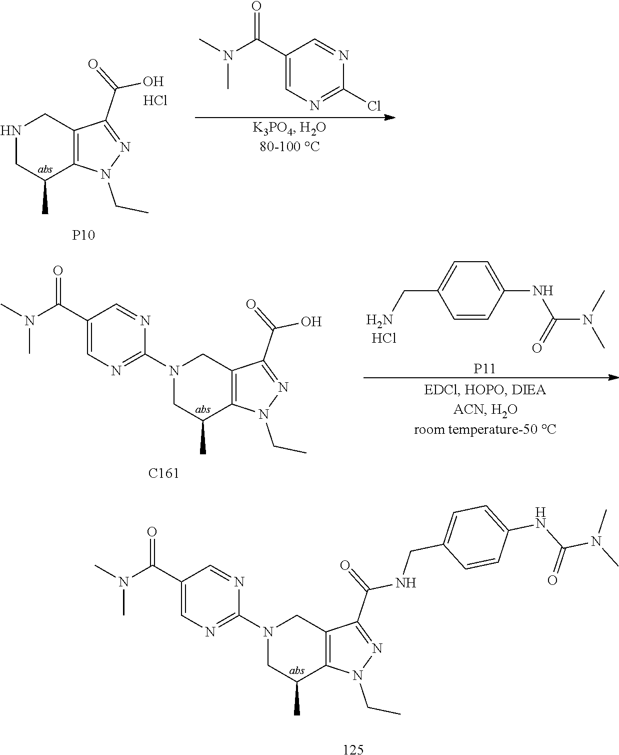

### Step 1. Preparation of (S)-5-(5-(dimethylcarbamoyl)pyrimidin-2-yl)-1-ethyl-7-methyl-4,5,6,7-tetrahydro-1H-pyrazolo[4,3-c]pyridine-3-carboxylic acid hydrochloride (C161)

To a solution of P10 (6.60 g, 26.9 mmol) and 2-chloro-N,N-dimethylpyrimidine-5-carboxamide (CAS: 1637587-62-2; 4.98 g, 26.9 mmol) in H₂O (130 mL) was added K3PO₄ (17.1 g, 80.6 mmol) at room temperature. The reaction mixture was stirred between 80-100 °C for 18 h and then cooled to room temperature. The suspension was extracted MeOH:DCM (100 mL), and then the organic layer was discarded. The aqueous layer was acidified to pH<5 with concentrated HCl and then extracted with (1:10) MeOH:DCM (150 mL×3). The combined organic layer was dried over Na₂SO₄ and concentrated in vacuo to provide C161 (8.20 g, crude) as a yellow solid. The solid was used directly in the next step without further purification. (LCMS) m/z (M+H)⁺=359.2. ¹H NMR (400 MHz, (CD₃)₂SO) δ (ppm): 12.74 (s, 1H), 8.53 (s, 2H), 5.38 (d, 1H), 4.69 (dd, 1H), 4.34 (d, 1H), 4.18-4.03 (m, 2H), 3.46 (dd, 1H), 3.28-3.19 (m, 1H), 2.99 (br s, 6H), 1.37 (t, 3H), 1.15 (d, 3H). The chiral purity was determined using Chiral HPLC analytical method on Chiralcel OJ-RH 150 mm×4.6 mm×5 μm column; Mobile phase A: H₂O (1.5 mL TFA)/Mobile phase B: ACN (1.5 mL TFA); 10-80% of Mobile phase B in 8 min then from 80-10% of Mobile phase B in 1 min and held at 10% of Mobile phase B for 6 min; flow rate: 0.8 mL/min; column temperature: 35 °C to provide C161: peak 1 (retention time: 7.659 min, 100% ee).

### Step 2. Preparation of (S)-5-(5-(dimethylcarbamoyl)pyrimidin-2-yl)-N-(4-(3,3-dimethylureido)benzyl)-1-ethyl-7-methyl-4,5,6,7-tetrahydro-1H-pyrazolo[4,3-c]pyridine-3-carboxamid (125)

To a solution of C161 (2.70 g, 6.84 mmol), EDCI (1.64 g, 8.55 mmol), and HOPO (0.950 g, 8.55 mmol) in ACN (35 mL) was added DIEA (2.21 g, 17.1 mmol). The solution was stirred at 50 °C. for 3.5 h, which caused precipitate to form. The reaction mixture was cooled to room temperature and stirred for 15 min. Then, P11 (1.65 g, 7.18 mmol), H₂O (0.123 g, 6.84 mmol), and an additional portion of DIEA (3.53 g, 27.3 mmol) were added. The reaction mixture was stirred at 50 °C for 2 h, diluted with EtOAc (50 mL), and washed with H₂O (2×50 mL). The aqueous layer was re-extracted with EtOAc (50 mL). The combined organic layer was washed with saturated NaHCO₃ (2×50 mL) and brine (50 mL). The organic layer was dried over Na₂SO₄, filtered, and concentrated in vacuo to give a yellow solid. The yellow solid was purified by column chromatography (silica gel; 0-5% MeOH: DCM) then lyophilized to give 125 (3.39 g, 92.9% yield) as a white solid. (LCMS) m/z (M+H)⁺=534.3. ¹H NMR (400 MHz, (CD₃)₂SO) δ (ppm): 8.52 (s, 2H), 8.45 (t, 1H), 8.23 (s, 1H), 7.41-7.35 (m, 2H), 7.19-7.13 (m, 2H), 5.41 (d, 1H), 4.67 (dd, 1H), 4.42-4.26 (m, 3H), 4.16-4.04 (m, 2H), 3.48 (dd, 1H), 3.28-3.19 (m, 1H), 2.99 (br s, 6H), 2.90 (s, 6H), 1.38 (t, 3H), 1.16 (d, 3H). The chiral purity was determined using SFC analytical method on Chiralcel OD-3 50 mm×4.6 mm×3 μm column; Mobile phase A: CO₂/Mobile phase B: EtOH with 0.2% of NH₃; gradient: 5-40% of Mobile Phase B in 1.5 min then held at 40% of Mobile phase B for 1.0 min then held at 5% of Mobile phase B for 0.5 min; backpressure: 1500 psi; flow rate: 4.0 mL/min; column temperature: 35 °C to provide 125: peak 1 ((SFC-MS) m/z (M+H)⁺=534.52 at retention time: 1.866 min, 100% ee). [α]²⁰_{D}=−154.134 (c=8.0 (g/L) in MeOH.

### Example 126

### (S)-5-(5-(Dimethylcarbamoyl)pyridin-2-yl)-N-(4-(3,3-dimethylureido)benzyl)-1-ethyl-7-methyl-4,5,6,7-tetrahydro-1H-pyrazolo[4,3-c]pyridine-3-carboxamide (126)

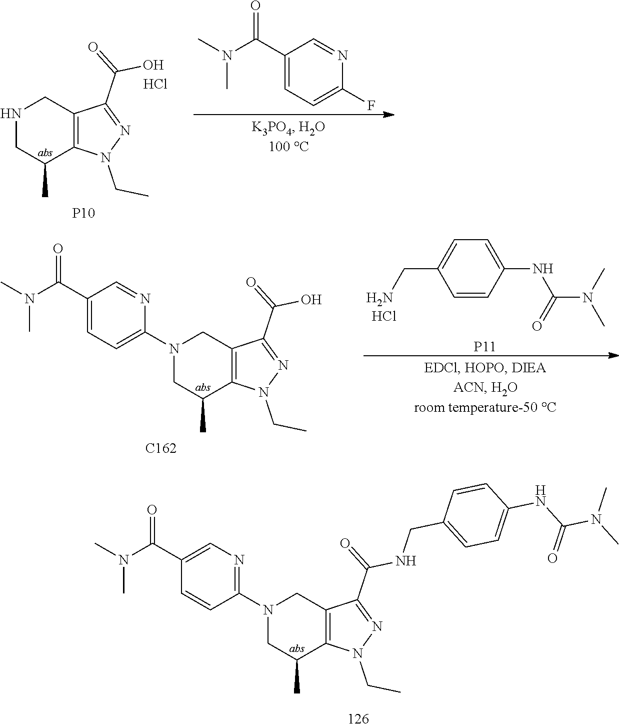

### Step 1. Preparation of (S)-5-(5-(dimethylcarbamoyl)pyridin-2-yl)-1-ethyl-7-methyl-4,5,6,7-tetrahydro-1H-pyrazolo[4,3-c]pyridine-3-carboxylic acid (C162)

To a solution of 6-fluoro-N,N-dimethylnicotinamide (CAS: 1032251-82-3; 3.61 g, 21.5 mmol) and P10 (5.56 g, 22.6 mmol) in H₂O (110 mL) was added K₃PO₄ (14.4 g, 67.9 mmol) at room temperature. The reaction mixture was stirred at 100 °C for 48 h and then cooled to room temperature. The suspension was extracted with MTBE (20 mL), and then the organic layer was discarded. The aqueous phase was acidified to pH-5-6 with concentrated HCl and then extracted with (1:10) MeOH:DCM (40 mL×3). The combined organic layer was dried over Na₂SO₄ and concentrated in vacuo to provide C162 (6.35 g, crude) as a yellow solid. The solid was used directly in the next step without further purification. (LCMS) m/z (M+H)⁺=358.0. ¹H NMR (400 MHz, CDCl₃) δ (ppm): 9.28 (br s, 1H), 8.43 (d, 1H), 7.71 (dd, 1H), 6.77 (d, 1H), 5.01 (d, 1H), 4.46-4.34 (m, 2H), 4.22-4.04 (m, 2H), 3.49-3.40 (m, 1H), 3.15-3.04 (m, 7H), 1.47 (t, 3H), 1.29 (d, 3H). The chiral purity was determined using SFC analytical method on Chiralcel OD-3 50 mm×4.6 mm×3 μm column; Mobile phase A: CO₂/Mobile phase B: EtOH with 0.2% of NH₃; gradient: 5-40% of Mobile Phase B in 1.5 min then held at 40% of Mobile phase B for 1.0 min then held at 5% of Mobile phase B for 0.5 min; backpressure: 1500 psi; flow rate: 4.0 mL/min; column temperature: 35 °C to provide C162: peak 1 ((SFC-MS) m/z (M+H)⁺=358.15 at retention time: 1.497 min, 100% ee).

### Step 2. Preparation of (S)-5-(5-(dimethylcarbamoyl)pyridin-2-yl)-N-(4-(3,3-dimethylureido)benzyl)-1-ethyl-7-methyl-4,5,6,7-tetrahydro-1H-pyrazolo[4,3-c]pyridine-3-carboxamide (126)

To a solution of C162 (3.00 g, 8.39 mmol), EDCI (2.01 g, 10.5 mmol), and HOPO (1.17 g, 10.5 mmol) in ACN (42 mL) was added DIEA (2.71 g, 21.0 mmol). The solution was stirred at 50 °C. for 2 h, which caused precipitate to form. The reaction mixture was cooled to room temperature and stirred for 15 min. Then, P11 (2.02 g, 8.81 mmol), H₂O (0.151 g, 8.39 mmol), and an additional portion of DIEA (4.34 g, 33.6 mmol) were added. The reaction mixture was stirred at 50 °C for 4 h, diluted with EtOAc (30 mL), and washed with H₂O (2×30 mL). The aqueous layer was re-extracted with EtOAc (10 mL). The combined organic layer was washed with saturated NaHCO₃ (2×30 mL) and brine (30 mL). The organic layer was dried over Na₂SO₄, filtered, and concentrated in vacuo to give a yellow solid. The solid was purified by column chromatography (silica gel; 0-10% MeOH: DCM) and then lyophilized to give 126 (2.20 g, 49.2% yield) as a yellow solid. (LCMS) m/z (M+H)⁺=533.4. ¹H NMR (400 MHz, (CD₃)₂SO) δ (ppm): 8.45 (t, 1H), 8.25-8.21 (m, 2H), 7.64 (dd, 1H), 7.41-7.35 (m, 2H), 7.19-7.13 (m, 2H), 6.89 (d, 1H), 5.05 (d, 1H), 4.41-4.25 (m, 4H), 4.17-4.04 (m, 2H), 3.43-3.36 (m, 1H), 3.26-3.17 (m, 1H), 2.98 (br s, 6H), 2.90 (s, 6H), 1.38 (t, 3H), 1.20 (d, 3H). The chiral purity was determined using SFC analytical method on Chiralcel OD-3 50 mm×4.6 mm×3 μm column; Mobile phase A: CO₂/Mobile phase B: EtOH with 0.2% of NH₃; Isocratic: 40% Mobile phase B; backpressure: 1500 psi; flow rate: 3.5 mL/min; column temperature: 35 °C to provide 126: peak 1 ((SFC-MS) m/z (M+H)⁺=533.61 at retention time: 1.530 min, 100% ee). [α]^{20D}=−121.791 (c=5.5 (g/L) in MeOH.

### Example 127

### (S)-5-(5-Dimethylcarbamoyl)pyrimidin-2-yl)-N-(4-(3,3-dimethylureido)benzyl)-1-isopropyl-7-methyl-4,5,6,7-tetrahydro-1H-pyrazolo[4,3-c]pyridine-3-carboxamide (127)

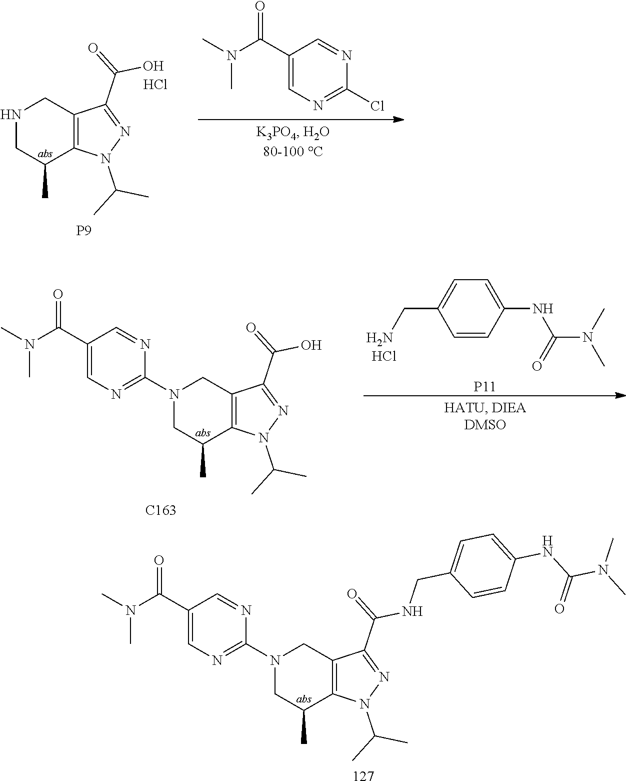

### Step 1. Preparation of (S)-5-(5-(dimethylcarbamoyl)pyrimidin-2-yl)-1-isopropyl-7-methyl-4,5,6,7-tetrahydro-1H-pyrazolo[4,3-c]pyridine-3-carboxylic acid (C163)

To a solution of 2-chloro-N,N-dimethylpyrimidine-5-carboxamide (CAS: 1637587-62-2; 3.86 g, 20.8 mmol) and P9 (5.40 g, 20.8 mmol) in H₂O (108 mL) was added K3PO₄ (13.2 g, 62.4 mmol) at room temperature. The reaction mixture was heated between 80-100 °C for 18 h and then cooled to room temperature. The reaction mixture was extracted with EtOAc (50 mL×2), and the combined organic layer was discarded. The aqueous layer was acidified to pH=5-6 with aqueous HCl and then extracted with (1:10) MeOH:DCM (50 mL×4). The combined organic layer was dried over Na₂SO₄ and concentrated in vacuo to give C163 (7.10 g, crude) as a light yellow solid. The solid was used directly in the next step without further purification. (LCMS) m/z (M+H)⁺=373.2. ¹H NMR (400 MHz, (CD₃)₂SO) δ (ppm): 12.73 (br s, 1H), 8.53 (s, 2H), 5.43 (d, 1H), 4.80-4.73 (m, 1H), 4.61-4.44 (m, 1H), 4.28 (d, 1H), 3.44-3.37 (m, 1H), 3.29-3.18 (m, 1H), 2.99 (br s, 6H), 1.40 (dd, 6H), 1.17-1.11 (m, 3H). The chiral purity was determined using SFC analytical method on Chiralpak AD-3 100 mm×4.6 mm×3 μm; Mobile phase A: CO₂/Mobile phase B: 40% EtOH with 0.2% of NH₃; backpressure: 1500 psi; flow rate: 2.8 mL/min; column temperature: 35 °C to provide C163: peak 1 ((SFC-MS) m/z (M+H)⁺=373.19 at retention time: 1.096 min, 100% ee).

### Step 2. Preparation of (S)-5-(5-(dimethylcarbamoyl)pyrimidin-2-yl)-N-(4-(3,3-dimethylureido)benzyl)-1-isopropyl-7-methyl-4,5,6,7-tetrahydro-1H-pyrazolo[4,3-c]pyridine-3-carboxamide (127)

To a solution of C163 (0.060 g, 0.16 mmol) in DMSO (1 mL) was added DIEA (0.062 g, 0.48 mmol), P11 (0.041 g, 0.18 mmol), and HATU (0.092 g, 0.24 mmol). The reaction was stirred at room temperature for 10 min and then was filtered. The filtrate was purified by reverse phase HPLC (Sunfire C₁₈ 100 mm×19 mm×5 μm column, Mobile Phase A: H₂O (0.05% TFA)/Mobile Phase B: ACN, 15 to 95% of Mobile Phase B over 9.0 min, 95% of Mobile Phase B hold 1.0 min, flow rate: 25 mL/min) to provide 127 (15 mg, 17% yield) as a solid. (LCMS) m/z (M+H)⁺=548.9. ¹H NMR (400 MHz, (CD₃)₂SO) δ (ppm): 8.52 (s, 2H), 8.34 (t, 1H), 8.23 (s, 1H), 7.41-7.36 (m, 2H), 7.19-7.14 (m, 2H), 5.47 (d, 1H), 4.75 (dd, 1H), 4.58-4.46 (m, 1H), 4.40-4.29 (m, 3H), 3.42 (dd, 1H), 3.29-3.23 (m, 1H) 3.04-2.94 (m, 6H), 2.91 (s, 6H), 1.38 (dd, 6H), 1.11 (d, 3H). The chiral purity was determined using SFC analytical method on Chiralpak AD-3 50 mm×4.6 mm×3 μm; Mobile phase A: CO₂/Mobile phase B: isopropyl alcohol with 0.2% NH₃; Isocratic: 40% Mobile phase B; backpressure: 1500 psi; flow rate: 3.5 mL/min; column temperature: 35 °C to provide 127: peak 1 ((SFC-MS) m/z (M+H)⁺=548.70 at retention time: 1.882 min, 100% ee). [α]²⁰=−149.239 (c=1.5 (g/L) in MeOH).

### Example 128

### (S)-5-(5-(Dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide (128)

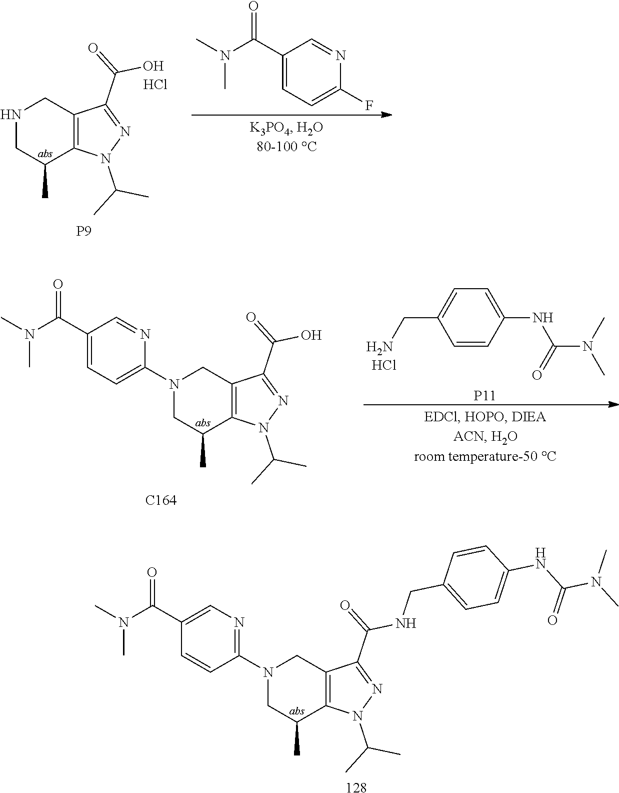

### Step 1. Preparation of (S)-5-(5-(dimethylcarbamoyl)pyridin-2-yl)-1-isopropyl-7-methyl-4,5,6,7-tetrahydro-1H-pyrazolo[4,3-c]pyridine-3-carboxylic acid (C164)

To a solution of 6-fluoro-N,N-dimethylnicotinamide (CAS: 1032251-82-3; 2.91 g, 17.3 mmol) and P9 (5.00 g, 19.2 mmol) in H₂O (100 mL) was added K₃PO₄ (12.3 g, 57.7 mmol) at room temperature. The reaction mixture was stirred between 80-100 °C for 18 h and then cooled to room temperature. To the suspension was added another portion of 6-fluoro-N,N-dimethylnicotinamide (CAS: 1032251-82-3; 0.227 g, 1.35 mmol), then the reaction mixture was stirred between 80-100 °C. for 48 h. The suspension was cooled to room temperature and extracted with EtOAc (100 mL×4). The combined organic layer was discarded. The aqueous layer was acidified to pH=5-6 with aqueous HCl then extracted with (1:10) MeOH:DCM (50 mL×9). The combined organic layer was dried over Na₂SO₄ and concentrated in vacuo to give C164 (5.80 g, crude) as a light yellow solid. The solid was used directly in the next step without further purification. (LCMS) m/z (M+H)⁺=372.1. ¹H NMR (400 MHz, (CD₃)₂SO) δ (ppm): 12.70 (br s, 1H), 8.26-8.24 (m, 1H), 7.64 (dd, 1H), 6.91 (d, 1H), 5.08 (d, 1H), 4.60-4.49 (m, 1H), 4.43-4.36 (m, 1H), 4.19 (d, 1H), 3.37-3.35 (m, 1H), 3.26-3.21 (m, 1H), 2.98 (s, 6H), 1.40 (dd, 6H), 1.20 (d, 3H). The chiral purity was determined using SFC analytical method on Chiralpak AD-3 100 mm×4.6 mm×3 μm; Mobile phase A: CO₂/Mobile phase B: 40% EtOH with 0.2% of NH₃; backpressure: 1500 psi; flow rate: 2.8 mL/min; column temperature: 35 °C to provide C164: peak 1 ((SFC-MS) m/z (M+H)⁺=372.17 at retention time: 1.254 min, 100% ee).

### Step 2. Preparation of (S)-5-(5-(dimethylcarbamoyl)pyridin-2-yl)-N-(4-(3,3-dimethylureido)benzyl)-1-isopropyl-7-methyl-4,5,6,7-tetrahydro-1H-pyrazolo[4,3-c]pyridine-3-carboxamide (128)

To a solution of C164 (3.20 g, 8.61 mmol), EDCI (2.06 g, 10.8 mmol), and HOPO (1.20 g, 10.8 mmol) in ACN (50 mL) was added DIEA (4.45 g, 34.4 mmol). The solution was stirred at 50 °C. for 2 h, which caused precipitate to form. The reaction mixture was cooled to room temperature, then P11 (2.18 g, 9.47 mmol), H₂O (0.155 g, 8.61 mmol), and an additional portion of DIEA (4.45 g, 34.4 mmol) were added. The reaction mixture was stirred at 50 °C for 6 h and then diluted with H₂O (20 mL). The suspension was concentrated in vacuo to remove the solvent and leave the aqueous layer. The aqueous layer was extracted with (1:10) MeOH:DCM (3×70 mL). The combined organic layer was washed with saturated NaHCO₃ (15 mL) and brine (15 mL). The organic layer was dried over Na₂SO₄, filtered, and concentrated in vacuo to give a yellow solid. The yellow solid was purified by column chromatography (silica gel; 0-10% MeOH: DCM) and then lyophilized to give 128 (1.75 g, 37.2% yield) as a white solid. (LCMS) m/z (M+H)⁺=547.3. ¹H NMR (400 MHz, (CD₃)₂SO) δ (ppm): 8.29 (t, 1H), 8.18-8.16 (m, 2H), 7.57 (dd, 1H), 7.34-7.29 (m, 2H), 7.14-7.08 (m, 2H), 6.82 (d, 1H), 5.03 (d, 1H), 4.50-4.41 (m, 1H), 4.39-4.23 (m, 3H), 4.17 (d, 1H), 3.36-3.35 (m, 1H), 3.18-3.12 (m, 1H), 2.91 (s, 6H), 2.84 (s, 6H), 1.35 (dd, 6H), 1.13 (d, 3H). The chiral purity was determined using SFC analytical method on Chiralcel OD-3 50 mm×4.6 mm×3 μm; Mobile phase A: CO₂/Mobile phase B: EtOH with 0.2% NH₃; Isocratic 40% of Mobile phase B; backpressure: 1500 psi; flow rate: 4.0 mL/min; column temperature: 35 °C to provide 128: peak 1 ((SFC-MS) m/z (M+H)⁺=547.54 at retention time: 1.035 min, 99% ee). [α]²⁵_{D}=−12.573 (c=2.6 (g/L) in MeOH).

### Example 132

### (S)-5-(4-(Dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide (132)

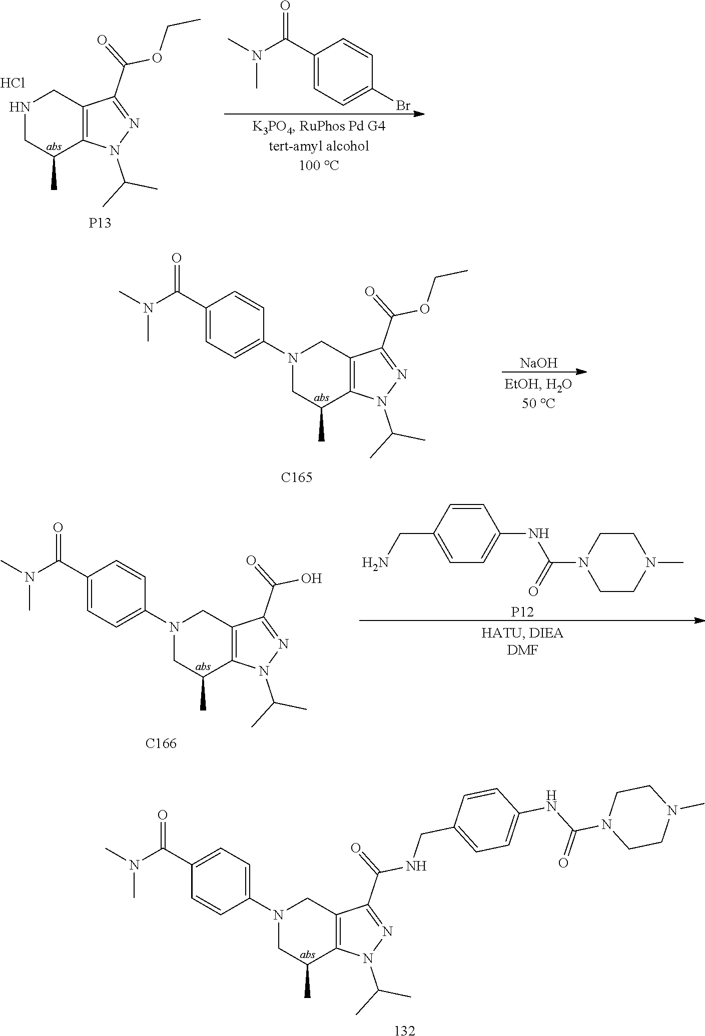

### Step 1. Preparation of ethyl (S)-5-(4-(dimethylcarbamoyl)phenyl)-1-isopropyl-7-methyl-4,5,6,7-tetrahydro-1H-pyrazolo[4,3-c]pyridine-3-carboxylate (C165)

A reaction mixture of 4-bromo-N,N-dimethylbenzamide (0.344 g, 1.51 mmol), K₃PO₄ (0.667 g, 3.14 mmol) and RuPhos Pd G4 (0.107 g, 0.126 mmol) was degassed with nitrogen gas before a solution of P13 (0.362 g, 1.26 mmol) in tert-amyl alcohol (3 mL) was added. The suspension was stirred at 100 °C for 25 h, then filtered and concentrated in vacuo. The residue was dissolved in DCM and purified by column chromatography (silica gel, 20-100% EtOAc: heptane) to provide C165 (0.512 g, crude) as a yellow oil. The oil was used directly in the next step without further purification. (LC/MS) m/z (M+H)⁺=399.6. ¹H NMR (600 MHz, (CD₃)₂SO) δ (ppm): 7.38-7.34 (m, 2H), 7.03-6.98 (m, 2H), 4.64-4.56 (m, 1H), 4.53 (d, 1H), 4.31 (q, 2H), 4.11 (d, 1H), 3.88-3.83 (m, 1H), 3.32-3.22 (m, 2H), 2.98 (s, 6H), 1.43 (dd, 6H), 1.34 (t, 3H), 1.31 (d, 3H).

### Step 2. Preparation of (S)-5-(4-(dimethylcarbamoyl)phenyl)-1-isopropyl-7-methyl-4,5,6,7-tetrahydro-1H-pyrazolo[4,3-c]pyridine-3-carboxylic acid (C166)

To a solution of C165 (0.500 g, 1.25 mmol) in EtOH (3 mL) was added NaOH (0.151 g, 3.76 mmol) and H₂O (1 mL). The reaction mixture was stirred at 50 °C for 25 h, then acidified with HCl and concentrated in vacuo to provide C166 (0.465 g, crude) as a yellow solid. The solid was used directly in the next step without further purification. (LC/MS) m/z (M+H)⁺=371.4. ¹H NMR (600 MHz, (CD₃)₂SO) δ (ppm): 7.37-7.31 (m, 2H), 7.01-6.95 (m, 2H), 4.61-4.52 (m, 1H), 4.50 (d, 1H), 4.11-4.05 (m, 1H), 3.82 (dd, 1H), 3.29 (dd, 1H), 3.24-3.18 (m, 1H), 2.96 (s, 6H), 1.44-1.39 (m, 6H), 1.29 (d, 3H).

### Step 3. Preparation of (S)-5-(4-(dimethylcarbamoyl)phenyl)-1-isopropyl-7-methyl-N-(4-(4-methylpiperazine-1-carboxamido)benzyl)-4,5,6,7-tetrahydro-1H-pyrazolo[4,3-c]pyridine-3-carboxamide (132)

To a solution of C166 (37 mg, 0.10 mmol) and P12 (32 mg, 0.10 mmol) in DMF (1 mL) was added HATU (38 mg, 0.10 mmol). The suspension was stirred at room temperature for 2 min, and then DIEA (64 mg, 0.50 mmol) was added. The yellow solution was stirred at room temperature for 2 h, then diluted with MeOH and purified by reverse phase HPLC (Phenomenex Gemini NX C₁₈ 150 mm×30 mm×5 μm column, Mobile Phase A: H₂O (NH₄OH—NH₄HCO₃)/Mobile Phase B: ACN, 26-66% of Mobile Phase B over 9 min, 100% of Mobile Phase B hold for 2 min, flow rate: 60 mL/min) and lyophilized to provide 132 (0.020 g, 34% yield) as a white solid. (LCMS) m/z (M+H)⁺=601.3. ¹H NMR (400 MHz, (CD₃)₂SO) δ 8.46 (s, 1H), 8.36 (t, 1H), 7.40-7.28 (m, 4H), 7.21-7.12 (m, 2H), 7.00-6.91 (m, 2H), 4.59-4.48 (m, 2H), 4.36-4.30 (m, 2H), 4.10 (d, 1H), 3.85-3.77 (m, 1H), 3.41 (t, 4H), 3.28-3.16 (m, 2H), 2.97-2.91 (m, 6H), 2.29 (t, 4H), 2.18 (s, 3H), 1.47-1.37 (m, 6H), 1.28 (d, 3H). The chiral purity was determined using SFC analytical method on Chiralcel OD-3 50 mm×4.6 mm×3 μm column; Mobile phase A: CO₂/Mobile phase B: EtOH (0.2% NH₃); 5 to 40% of Mobile phase B in 1.5 min and held at 40% of Mobile phase B for 1.0 min then 5% of Mobile phase B for 0.5 min; backpressure: 1500 psi; flow rate: 4.0 mL/min; column temperature: 35 °C to provide peak 1: ((SFC-MS) m/z (M+H)⁺=601.32 at retention time: 1.830 min, 0.27% ee) and 132: peak 2 ((SFC-MS) m/z (M+H)⁺=601.31 at retention time: 2.105 min, 99.73% ee).

### Example 136

### 5-(5-(Dimethylcarbamoyl)pyridin-2-yl)-1-isopropyl-N-(4-(pyrrolidine-1-carboxamido)benzyl)-4,5,6,7-tetrahydro-1H-pyrazolo[4,3-c]pyridine-3-carboxamide (136)

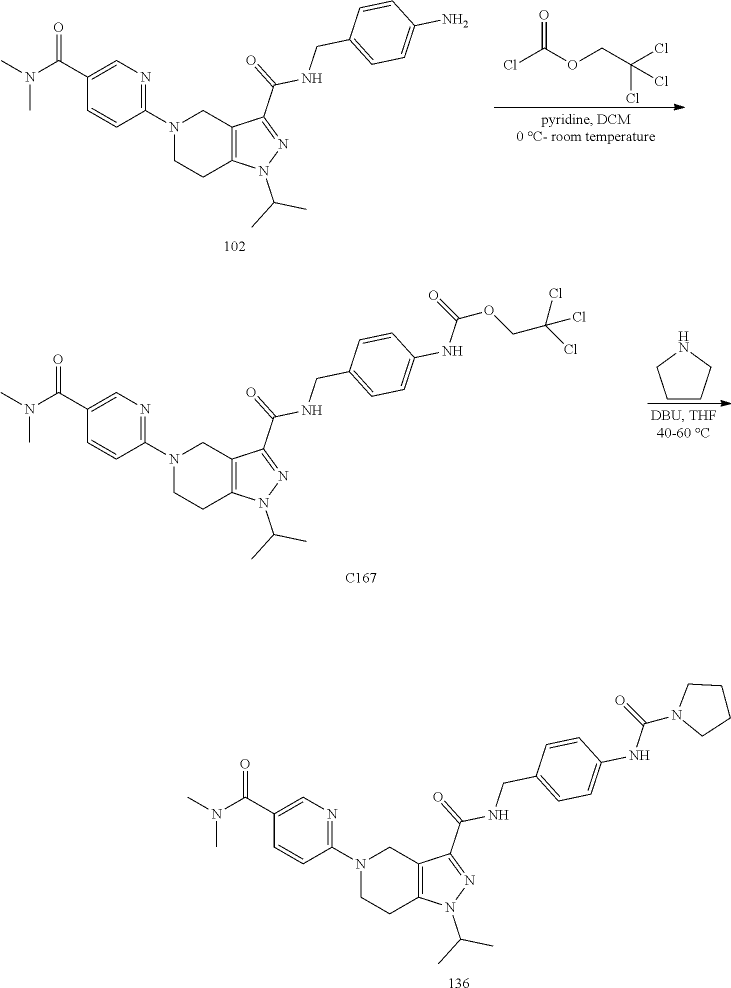

### Step 1. Preparation of 2,2,2-trichloroethyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (C167)

At 0 °C, to a solution of 102 (1.3 g, 2.8 mmol) in DCM (13 mL) was added pyridine (0.49 g, 6.2 mmol) followed by a solution of 2,2,2-trichloroethoxycarbonylchloride (0.77 g, 3.6 mmol) in DCM (1 mL) dropwise. The reaction mixture was stirred for 30 min at 0 °C, then warmed to room temperature and concentrated in vacuo. The residue was dissolved in MeOH: DCM then purified by column chromatography (silica gel, 0-10% MeOH: DCM) to provide C167 (1.7 g, 93% yield) as a white foam. (LCMS) m/z (M+H)⁺=638.6.

### Step 2. Preparation of 5-(5-(dimethylcarbamoyl)pyridin-2-yl)-1-isopropyl-N-(4-(pyrrolidine-1-carboxamido)benzyl)-4,5,6,7-tetrahydro-1H-pyrazolo[4,3-c]pyridine-3-carboxamide (136)

In a gold Para-Dox™ plate was added a reaction mixture of pyrrolidine (0.070 mL) and DBU (0.030 mL) in THF (0.25 mL) and then shaken at 40 °C for 2 h. A solution of C167 (6.4 mg, 0.010 mmol) in THF (0.023 mL) was added to the suspension, and then the reaction mixture was sealed and shaken at 60 °C overnight. The suspension was dissolved in (1:1, MeOH: DCE, 0.35 mL) and then filtered. The filter cake was rinsed with (1:1, MeOH: DCE, 0.35 mL×2). The filtrate was concentrated in vacuo and then dissolved in (1:1, MeOH: DCE, 0.20 mL) and shaken at room temperature for 2 h. The solution was purified by reverse phase HPLC (C₁₈ 50 mm×10 mm×5 μm column; Mobile Phase A: H₂O/Mobile Phase B: ACN (0.05% formic acid), 5 to 95% of Mobile Phase B over 5 min, flow rate: 4.0 mL/min) to provide 136 (1.1 mg, 20% yield). (LCMS) m/z (M+H)⁺=559.4.

### Example 193

### (S)-5-(5-(3-Acetamidopyrrolidine-1-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide (193)

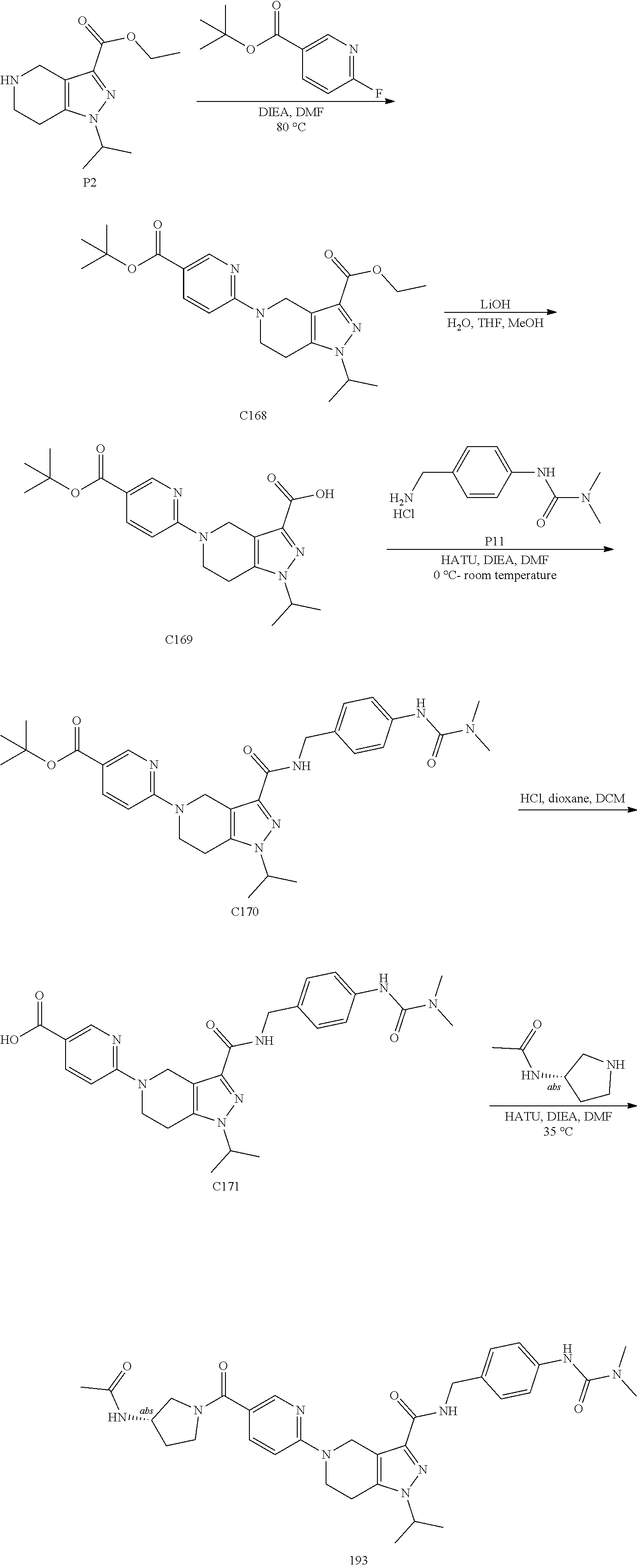

### Step 1. Preparation of ethyl 5-(5-(tert-butoxycarbonyl)pyridin-2-yl)-1-isopropyl-4,5,6,7-tetrahydro-1H-pyrazolo[4,3-c]pyridine-3-carboxylate (C168)

A suspension of P2 (1.85 g, 6.76 mmol) in DMF (40 mL) was added DIEA (2.62 g, 20.3 mmol) and tert-butyl 6-fluoronicotinate (CAS: 676560-01-3, 1.33 g, 6.76 mmol). The reaction mixture was stirred at 80 °C for 16 h, and then an additional portion of tert-butyl 6-fluoronicotinate (CAS: 676560-01-3,133 mg, 0.676 mmol) was added. The reaction mixture was stirred at 80 °C for 4 h and then was diluted with H₂O (300 mL). The suspension was extracted with EtOAc (3×100 mL). The combined organic layer was washed with brine (2×100 mL), dried with Na₂SO₄, filtered, and concentrated in vacuo. The residue was purified by column chromatography (silica gel, 0-25% EtOAc: PE) to provide C168 (1.86 g, 66.4%) as a white solid.

### Step 2. Preparation of 5-(5-(tert-butoxycarbonyl)pyridin-2-yl)-1-isopropyl-4,5,6,7-tetrahydro-1H-pyrazolo[4,3-c]pyridine-3-carboxylic acid (C169)

The same reaction was conducted in two batches and then combined for acidification. To a solution of C168 (1.76 g, 4.25 mmol) in THF (10 mL), MeOH (5 mL) and H₂O (10 mL) was added LiOH (0.445 g, 10.6 mmol) to form the first batch then stirred for 16 h.

A second batch of the same reaction was conducted with C168 (0.100 g, 0.241 mmol). The two batches were combined and then concentrated in vacuo. The residue was diluted with H₂O (15 mL) and acidified with 1 M HCl to pH-4-5, which caused precipitate to form. The suspension was filtered, and the filter cake was washed with H₂O (2×10 mL). The filter cake was collected and dried by high vacuum to provide C169 (1.73 g, crude) as an off-white solid. The solid was used directly in the next step without further purification. (LCMS) m/z (M+H)⁺=387.1.

### Step 3. Preparation of tert-butyl 6-(3-((4-(3,3-dimethylureido)benzyl)carbamoyl)-1-isopropyl-1,4,6,7-tetrahydro-5H-pyrazolo[4,3-c]pyridin-5-yl)nicotinate (C170)

At 0 °C, to a solution of C169 (1.73 g, 4.49 mmol) in DMF (30 mL) was added HATU (1.88 g, 4.94 mmol) and stirred for 20 min; then, DIEA (2.32 g, 17.9 mmol) and P11 (1.13 g, 4.94 mmol) were added. The reaction mixture was stirred at room temperature for 16 h and then diluted with H₂O (200 mL). The suspension was extracted with (1:10, MeOH: DCM, 3×100 mL). The combined organic layer was washed with brine (2×150 mL), dried with Na₂SO₄, filtered, and concentrated in vacuo. The residue was purified by column chromatography (silica gel, 0-5% MeOH: DCM) to provide impure C170 (4.00 g), which was re-purified again by column chromatography (silica gel, 0-2.5% MeOH: DCM) to provide C170 (3.5 g, >95.0% yield) as a white solid. (LCMS) m/z (M+H)⁺=562.2. ¹H NMR (400 MHz, (CD₃)₂SO) δ 8.62 (d, 1H), 8.35 (t, 1H), 8.26-8.21 (m, 1H), 7.92 (dd, 1H), 7.44-7.36 (m, 2H), 7.21-7.14 (m, 2H), 6.91 (d, 1H), 4.80 (s, 2H), 4.54-4.43 (m, 1H), 4.35 (d, 2H), 4.04-3.94 (m, 2H), 2.96-2.89 (m, 6H), 2.86-2.79 (m, 2H), 1.52 (s, 9H), 1.40 (d, 6H).

### Step 4. Preparation of 6-(3-((4-(3,3-dimethylureido)benzyl)carbamoyl)-1-isopropyl-1,4,6,7-tetrahydro-5H-pyrazolo[4,3-c]pyridin-5-yl)nicotinic acid (C171)

To a solution of C170 (3.50 g, 6.23 mmol) in DCM (70 mL) was added 2M HCl in dioxane (35 mL) and then stirred for 16 h, and an additional portion of 2M HCl in dioxane (30 mL) was added. The reaction mixture was stirred for 2 h and then was diluted with H₂O (20 mL) and concentrated in vacuo to remove the organic layer. The aqueous layer was basified with NaHCO₃ until pH=5, which caused white precipitation to form. The mixture was filtered, and then the filter cake was washed with H₂O (2×30 mL), dried under a high vacuum, and then lyophilized to provide C171 (2.22 g, 69.8% yield) as a white solid (LCMS) m/z (M+H)⁺=506.4. ¹H NMR (400 MHz, (CD₃)₂SO) δ 12.51 (s, 1H), 8.65 (d, 1H), 8.35 (t, 1H), 8.23 (s, 1H), 7.96 (dd, 1H), 7.42-7.36 (m, 2H), 7.20-7.14 (m, 2H), 6.91 (d, 1H), 4.80 (s, 2H), 4.58-4.43 (m, 1H), 4.34 (d, 2H), 4.04-3.97 (m, 2H), 2.91 (s, 6H), 2.86-2.80 (m, 2H), 1.39 (d, 6H).

### Step 5. Preparation of (S)-5-(5-(3-acetamidopyrrolidine-1-carbonyl)pyridin-2-yl)-N-(4-(3,3-dimethylureido)benzyl)-1-isopropyl-4,5,6,7-tetrahydro-1H-pyrazolo[4,3-c]pyridine-3-carboxamide (193)

A solution of (S)—N-(pyrrolidin-3-yl)acetamide (CAS: 114636-31-6; 1.9 mg, 15 μmol) in (1:9, DIEA: DMF, 5.0 μL: 45 μL) was shaken at 30 °C for 1.5 h.

In a gold Para-Dox© plate, a solution of C171 (66 mg, 0.13 mmol), HATU (74 mg, 0.19 mmol), and DIEA (0.065 mL) in DMF (0.58 mL) was stirred for 20 min and then shaken at 35 °C for 19 h. The solution was concentrated under nitrogen gas. The residue was dissolved in (1:1, MeOH: DCE) and then shaken at 30 °C for 10 min. The suspension was filtered, and then the filter cake was rinsed with (1:1, MeOH: DCE, 0.60 mL). The filtrate was concentrated with nitrogen gas. The residue was dissolved in DMSO (0.15 mL) and then shaken at 30 °C for 30 min. TFA (2.0 μL) and H₂O (0.50 mL) were added to the suspension. The suspension was concentrated with nitrogen gas then dissolved in DMSO (0.15 mL) and purified by reverse phase HPLC (C₁₈ 50 mm×10 mm×5 μm column; Mobile Phase A: H₂O/Mobile Phase B: ACN (0.05% formic acid), 50 to 80% of Mobile Phase B over 5 min, flow rate: 4.0 mL/min) to provide 193 (2.1 mg, 34% yield). (LCMS) m/z (M+H)⁺=616.4.

### Example 236

### Ethyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (236)

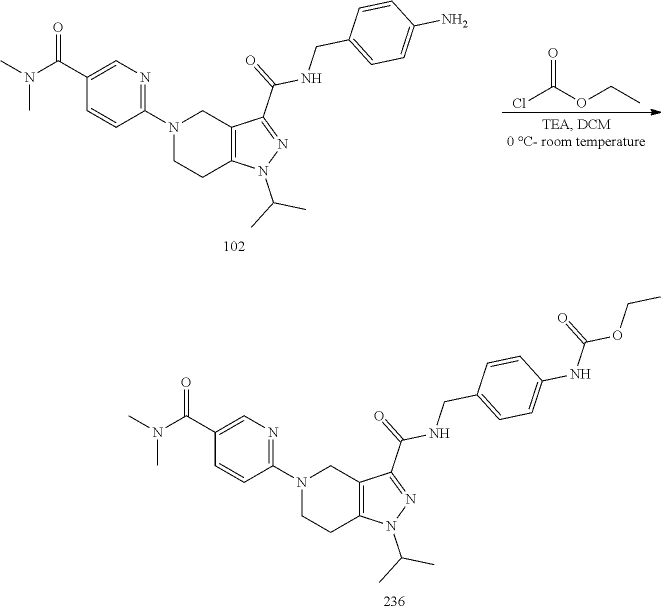

### Step 1. Preparation of ethyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (236)

At 0 °C, to a reaction mixture of 102 (0.200 g, 0.433 mmol) in DCM (2 mL) was added TEA (0.132 g, 1.30 mmol), then a solution of ethyl carbonochloridate (CAS: 541-41-3; 0.310 g, 2.86 mmol) in DCM (1 mL) dropwise. The reaction mixture was sealed and stirred at room temperature for 16 h. The suspension was diluted with H₂O (10 mL) and stirred at 0 °C for 30 min, and then the mixture's pH was adjusted to pH=8. The reaction mixture was extracted with DCM (2×10 mL). The organic layers were combined, dried with Na₂SO₄, and concentrated in vacuo. The residue was purified by reverse phase HPLC (Welch Xtimate C₁₈ 150 mm×25 mm×5 μm column, Mobile Phase A: H₂O (NH₄OH—NH₄HCO₃)/Mobile Phase B: ACN, 38 to 58% of Mobile Phase B over 11 min, 100% of Mobile Phase B hold 2 min, flow rate: 60 mL/min) and lyophilized to provide 236 (84.0 mg, 36.4% yield) as a white solid. (LCMS) m/z (M+H)⁺=534.3. ¹H NMR (400 MHz, (CD₃)₂SO) δ 9.46 (s, 1H), 8.28 (t, 1H), 8.16-8.14 (m, 1H), 7.55 (dd, 1H), 7.30 (d, 2H), 7.14 (d, 2H), 6.80 (d, 1H), 4.66 (s, 2H), 4.46-4.36 (m, 1H), 4.27 (d, 2H), 4.02 (q, 2H), 3.86 (t, 2H), 2.88 (s, 6H), 2.73 (t, 2H), 1.31 (d, 6H), 1.14 (t, 3H).

### Example 252

### 6-(10-(4-(3,3-Dimethylureido)benzyl)-11-oxo-1,3,4,7,8,9,10,11-octahydro-2H-pyrido[4′,3′:3,4]pyrazolo[1,5-a][1,4]diazepin-2-yl)-N,N-dimethylnicotinamide (252)

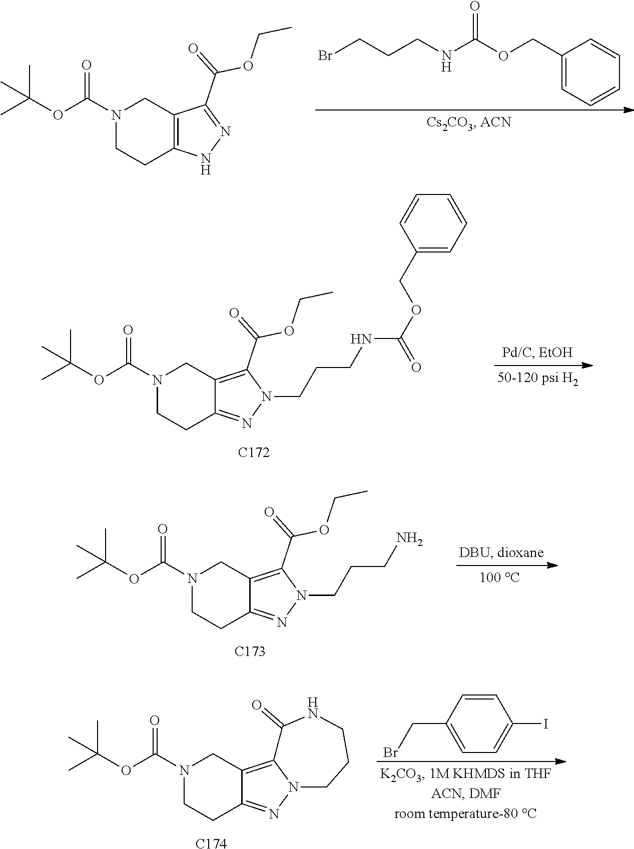

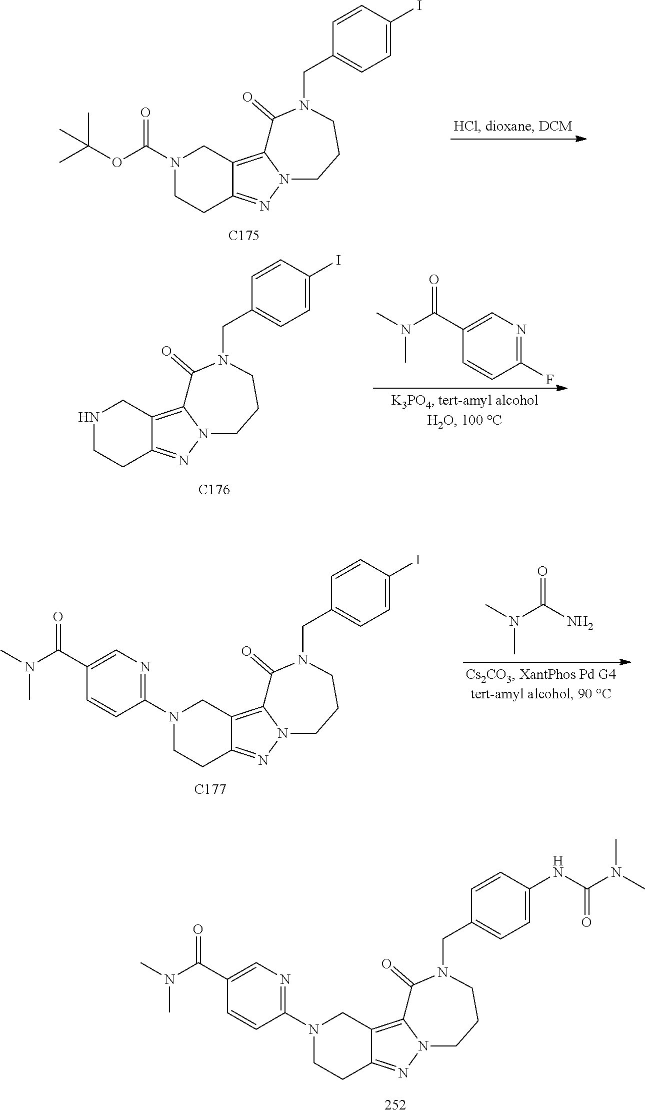

### Step 1. Preparation of 5-(tert-butyl) 3-ethyl 2-(3-(((benzyloxy)carbonyl)amino)propyl)-2,4,6,7-tetrahydro-5H-pyrazolo[4,3-c]pyridine-3,5-dicarboxylate (C172)

A reaction mixture of 5-(tert-butyl) 3-ethyl 1,4,6,7-tetrahydro-5H-pyrazolo[4,3-c]pyridine-3,5-dicarboxylate (CAS: 518990-23-3; 1.00 g, 3.39 mmol), benzyl (3-bromopropyl)carbamate (CAS: 39945-54-5; 967 mg, 3.56 mmol) and Cs₂CO₃ (1.65 g, 5.08 mmol) in ACN (20 mL) was stirred at room temperature for -72 h. The reaction mixture was filtered, and the filtrate was concentrated in vacuo. The residue was dissolved in DCM and then purified by column chromatography (silica gel, 0-60% EtOAc: heptane) to provide C172 (1.02 g, 61.9% yield) as a clear oil. (LCMS) m/z (M+H)⁺=487.3. ¹H NMR (600 MHz, (CD₃)₂SO) δ 7.41-7.26 (m, 6H), 5.01 (s, 2H), 4.53 (s, 2H), 4.45 (t, 2H), 4.32-4.28 (m, 2H), 3.60 (t, 2H), 3.01 (q, 2H), 2.64 (t, 2H), 1.93-1.85 (m, 2H), 1.44-1.41 (m, 9H), 1.32 (t, 3H).

### Step 2. Preparation of 5-(tert-butyl) 3-ethyl 2-(3-aminopropyl)-2,4,6,7-tetrahydro-5H-pyrazolo[4,3-c]pyridine-3,5-dicarboxylate (C173)

Pd/C (10 wt %, 0.153 g, 1.44 mmol) and C172 (1.00 g, 2.06 mmol) were added to a pressure vessel in EtOH (10 mL). The reaction mixture was stirred at 50 psi of H₂ gas for -24 h. An additional portion of Pd/C (10 wt %, 0.153 g, 1.44 mmol) was added and stirred at 120 psi of H₂ gas for ~24 h. The reaction mixture was filtered through celite, and then the filtrate was concentrated in vacuo to provide C173 (0.745 g, crude) as a clear oil. The oil was used directly in the next step without further purification. ¹H NMR (400 MHz, (CD₃)₂SO) δ 4.55-4.41 (m, 3H), 4.37-4.26 (m, 3H), 3.60 (t, 2H), 3.50-3.40 (m, 2H), 2.64 (t, 2H), 2.57-2.52 (m, 2H), 1.92-1.77 (m, 2H), 1.44-1.41 (m, 9H), 1.33 (t, 3H).

### Step 3. Preparation of tert-butyl 11-oxo-1,3,4,7,8,9,10,11-octahydro-2H-pyrido[4′,3′:3,4]pyrazolo[1,5-a][1,4]diazepine-2-carboxylate (C174)

To a solution of C173 (0.724 g, 2.05 mmol) in dioxane (10 mL) was added DBU (0.109 g, 0.719 mmol) and then stirred at 100 °C for 23 h. The reaction mixture was concentrated in vacuo to provide C174 (0.820 g, crude) as a yellow oil. The oil was used directly in the next step without further purification. ¹H NMR (400 MHz, (CD₃)₂SO) δ 7.89 (s, 1H), 4.24-4.20 (m, 2H), 4.09 (t, 2H), 3.03-2.87 (m, 4H), 2.42-2.33 (m, 2H), 1.91-1.83 (m, 2H), 1.19-1.13 (m, 9H).

### Step 4. Preparation of tert-butyl 10-(4-iodobenzyl)-11-oxo-1,3,4,7,8,9,10,11-octahydro-2H-pyrido[4′,3′:3,4]pyrazolo[1,5-a][1,4]diazepine-2-carboxylate (C175)

To a solution of C174 (0.629 g, 2.05 mmol) in ACN (10 mL) was added 1-(bromomethyl)-4-iodobenzene (CAS: 589-87-7; 0.610 g, 2.05 mmol) and K₂CO₃ (426 mg, 3.08 mmol). The reaction mixture was stirred at 80 °C for 2 h and dissolved in THF (10 mL). The solution was treated with 1M KHMDS in THF (2.0 mL) at room temperature and then diluted with DMF (1 mL). The reaction mixture was stirred at room temperature for 2.5 h and then concentrated in vacuo. The residue was suspended with H₂O and extracted with EtOAc. The organic layer was concentrated in vacuo, and then the residue was dissolved in DCM. The suspension was purified by column chromatography (silica gel, 0-80% EtOAc: heptane) to provide C175 (0.496 g, 46.3% yield) as a white foam. ¹H NMR (400 MHz, (CD₃)₂SO) δ 7.76-7.64 (m, 2H), 7.22-7.13 (m, 2H), 4.62 (s, 2H), 4.47 (s, 2H), 4.26 (t, 2H), 3.60 (t, 2H), 3.39-3.34 (m, 2H), 2.62 (t, 2H), 2.07-1.98 (m, 2H), 1.42 (s, 9H).

### Step 5. Preparation of 10-(4-iodobenzyl)-1,2,3,4,7,8,9,10-octahydro-11H-pyrido[4′,3′:3,4]pyrazolo[1,5-a][1,4]diazepin-11-one hydrochloride (C176)

To a solution of C175 (0.496 g, 0.949 mmol) in DCM (10 mL) was added 4M HCl in dioxane (0.95 mL). The reaction mixture was concentrated in vacuo to provide C176 (0.436 g, crude) as a white solid. The solid was used directly in the next step without further purification. (LCMS) m/z (M+H)⁺=423.3. ¹H NMR (400 MHz, (CD₃)₂SO) δ 9.12 (s, 1H), 7.75-7.69 (m, 2H), 7.21-7.14 (m, 2H), 4.65-4.60 (m, 2H), 4.35-4.29 (m, 2H), 4.27-4.21 (m, 2H), 3.43-3.34 (m, 4H), 2.91-2.83 (m, 2H), 2.12-2.01 (m, 2H).

### Step 6. Preparation of 6-(10-(4-iodobenzyl)-11-oxo-1,3,4,7,8,9,10,11-octahydro-2H-pyrido[4′,3′:3,4]pyrazolo[1,5-a][1,4]diazepin-2-yl)-N,N-dimethylnicotinamide (C177)

To a mixture of C176 (0.436 g, 0.950 mmol), K₃PO₄ (0.605 g, 2.85 mmol), H₂O (4 mL), and tert-amyl alcohol (4 mL), 6-fluoro-N,N-dimethylnicotinamide (CAS: 1032251-82-3; 176 mg, 1.05 mmol) was added. The reaction mixture was stirred at 100 °C for -72 h, diluted with H₂O, and extracted with EtOAc. The organic layer was washed with H₂O and concentrated in vacuo to provide C177 (0.632 g, crude) as a yellow oil. The oil was used directly in the next step without further purification.

### Step 7. Preparation of 6-(10-(4-(3,3-dimethylureido)benzyl)-11-oxo-1,3,4,7,8,9,10,11-octahydro-2H-pyrido[4′,3′:3,4]pyrazolo[1,5-a][1,4]diazepin-2-yl)-N,N-dimethylnicotinamide (252)

A reaction mixture of C177 (0.080 g, 0.14 mmol), 1,1-dimethylurea (CAS: 598-94-7; 37 mg, 0.42 mmol), Cs₂CO₃ (0.14 g, 0.42 mmol) and XantPhos Pd G4 (13 mg, 0.014 mmol) was degassed with nitrogen gas then tert-amyl alcohol (1 mL) was added. The suspension was stirred at 90 °C for 2 h and then filtered, and the filtrate was concentrated in vacuo. The residue was suspended in DMSO, filtered and purified by reverse phase HPLC (Sunfire C₁₈ 100 mm×19 mm×5 μm column, Mobile Phase A: H₂O/Mobile Phase B: ACN (0.05% TFA), 5 to 95% of Mobile Phase B over 10 min, flow rate: 25 mL/min) to provide 252 (4.6 mg, 6.2% yield). (LC/MS) m/z (M+H)⁺=531.4. ¹H NMR (600 MHz, (CD₃)₂SO) δ 8.28 (s, 1H), 8.24 (d, 1H), 7.64 (dd, 1H), 7.48-7.44 (m, 2H), 7.27-7.23 (m, 2H), 6.92 (d, 1H), 4.74 (s, 2H), 4.60 (s, 2H), 4.24 (t, 2H), 3.94 (t, 2H), 3.33 (t, 2H), 2.97 (s, 6H), 2.92 (s, 6H), 2.73 (t, 2H), 1.99-1.92 (m, 2H).

### Example 286

### 5-(5-(2-(Dimethylamino)-2-oxoethyl)pyridin-2-yl)-N-(4-(3,3-dimethylureido)benzyl)-1-isopropyl-4,5,6,7-tetrahydro-1H-pyrazolo[4,3-c]pyridine-3-carboxamide (286)

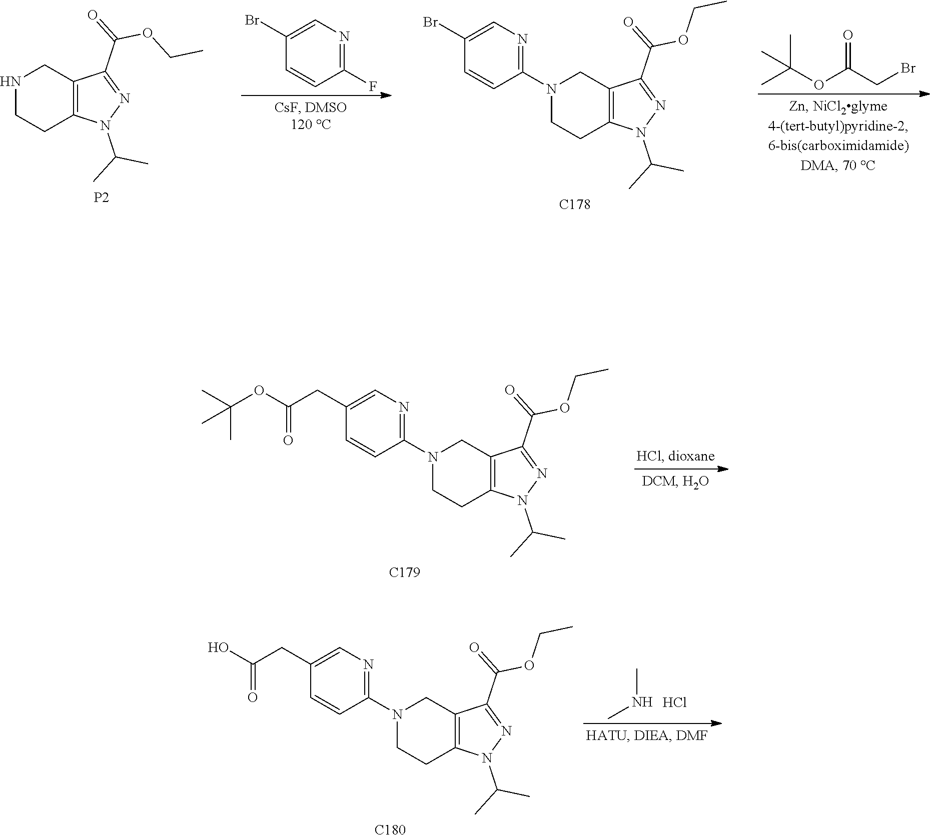

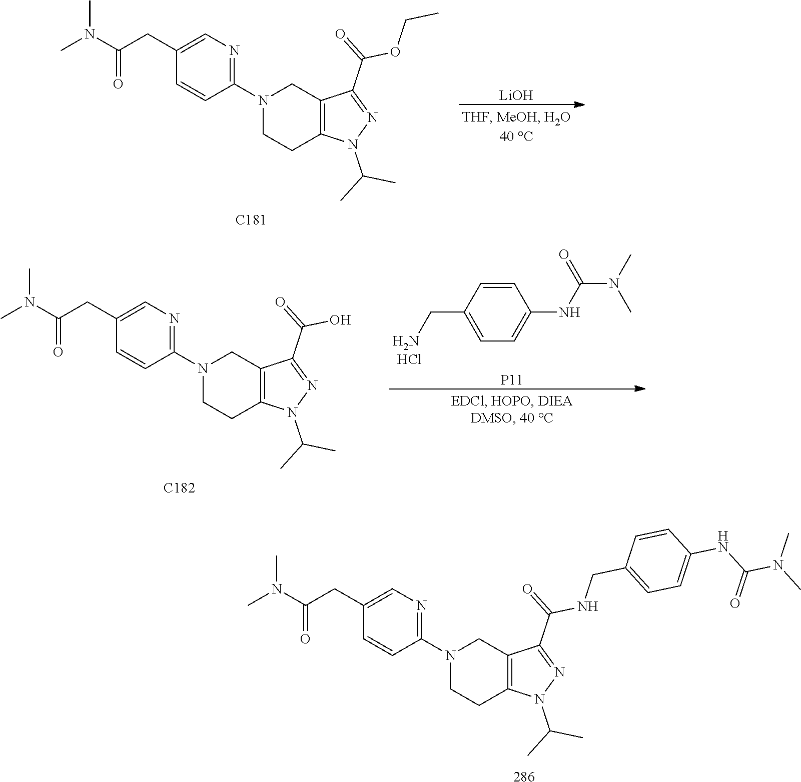

### Step 1. Preparation of ethyl 5-(5-bromopyridin-2-yl)-1-isopropyl-4,5,6,7-tetrahydro-1H-pyrazolo[4,3-c]pyridine-3-carboxylate (C178)

To a solution of P2 (4.10 g, 15.0 mmol) and CsF (5.69 g, 37.4 mmol) in DMSO (50 mL) was added 5-bromo-2-fluoropyridine (CAS: 766-11-0; 2.64 g, 15.0 mmol). The reaction mixture was stirred at 120 °C for 5 h. The solution was diluted with H₂O (40 mL) and extracted with EtOAc (3×50 mL). The organic layer was washed with brine (50 mL), dried with Na₂SO₄, and concentrated in vacuo. The residue was purified by column chromatography (silica gel, 0-25% EtOAc: PE) to provide C178 (3.20 g, 54.3% yield) as a yellow solid. ¹H NMR (400 MHz, (CD₃)₂SO) δ 8.18 (d, 1H), 7.70 (dd, 1H), 6.90 (d, 1H), 4.64 (s, 2H), 4.56-4.45 (m, 1H), 4.28 (q, 2H), 3.87 (t, 2H), 2.80 (t, 2H), 1.36 (d, 6H), 1.30 (t, 3H).

### Step 2. Preparation of ethyl 5-(5-(2-(tert-butoxy)-2-oxoethyl)pyridin-2-yl)-1-isopropyl-4,5,6,7-tetrahydro-1H-pyrazolo[4,3-c]pyridine-3-carboxylate (C179)

Under nitrogen gas, to C178 (0.58 g, 1.5 mmol) was added tert-butyl bromoacetate (CAS: 1211526-62-3; 0.29 g, 1.5 mmol), Zn (0.27 g, 3.7 mmol), NiCl₂·glyme (32 mg, 0.15 mmol), 4-(tert-butyl)pyridine-2,6-bis(carboximidamide) (CAS: 3003061-14-8; 43 mg, 0.15 mmol) and DMA (10 mL). The solution was degassed with nitrogen gas for 2 min and then stirred at 70 °C for 16 h. The reaction mixture was filtered, and the filtrate was concentrated in vacuo. The residue was diluted with EtOAc (20 mL) and then washed with H₂O (3×10 mL) followed by brine (2×10 mL). The organic layer was dried over Na₂SO₄, filtered, and concentrated in vacuo. The residue was purified by column chromatography (silica gel, 0-30% EtOAc: PE) to provide C179 (87 mg, 14% yield) as a colorless oil. (LC/MS) m/z (M+H)⁺=429.2.

### Step 3. Preparation of 2-(6-(3-(ethoxycarbonyl)-1-isopropyl-1,4,6,7-tetrahydro-5H-pyrazolo[4,3-c]pyridin-5-yl)pyridin-3-yl)acetic acid (C180)

To a solution of C179 (87 mg, 0.20 mmol) in DCM (3.0 mL), 2M HCl in dioxane (0.20 g, 6.0 mmol) and H₂O (0.1 mL) were added. The reaction mixture was stirred at room temperature for 2 h and then concentrated in vacuo to provide C180 (76 mg, crude) as a colorless oil. The oil was used directly in the next step without further purification.

### Step 4. Preparation of ethyl 5-(5-(2-(dimethylamino)-2-oxoethyl)pyridin-2-yl)-1-isopropyl-4,5,6,7-tetrahydro-1H-pyrazolo[4,3-c]pyridine-3-carboxylate (C181)

To a solution of C180 (78 mg, 0.21 mmol) in DMF (5.0 mL) was added HATU (0.13 g, 0.34 mmol), DIEA (0.15 g, 1.1 mmol), and dimethylamine hydrochloride (CAS: 506-59-2; 28 mg, 0.34 mmol). The reaction mixture was stirred at room temperature for 1 h, diluted with H₂O (4 mL), and extracted with EtOAc (3×5 mL). The organic layer was washed with brine (5 mL), dried over Na₂SO₄, and concentrated in vacuo. The residue was purified by column chromatography (silica gel, 0-80% EtOAc: PE) to provide C181 (0.12 g, 87% yield) as a yellow oil. (LC/MS) m/z (M+H)⁺=400.3.

### Step 5. Preparation of 5-(5-(2-(dimethylamino)-2-oxoethyl)pyridin-2-yl)-1-isopropyl-4,5,6,7-tetrahydro-1H-pyrazolo[4,3-c]pyridine-3-carboxylic acid (C182)

To a solution of C181 (0.12 g, 0.30 mmol) in THF (4.0 mL), MeOH (0.5 mL), and H₂O (1.0 mL) was added LiOH (22 mg, 0.90 mmol). The reaction mixture was then stirred at 40 °C for 3 h then was purified by reverse phase HPLC (C₁₈ 150 mm×30 mm×5 μm column, Mobile Phase A: H₂O (0.05% formic acid)/Mobile Phase B: ACN, 0 to 34% of Mobile Phase B over 9 min, 100% of Mobile Phase B hold 2 min, flow rate: 30 mL/min) and lyophilized to provide C182 (35 mg, 31% yield) as a white solid.

### Step 6. Preparation of 5-(5-(2-(dimethylamino)-2-oxoethyl)pyridin-2-yl)-N-(4-(3,3-dimethylureido)benzyl)-1-isopropyl-4,5,6,7-tetrahydro-1H-pyrazolo[4,3-c]pyridine-3-carboxamide (286)

To a solution of C182 (35 mg, 0.094 mmol) in DMSO (3.0 mL) was added DIEA (61 mg, 0.47 mmol), EDCI (43 mg, 0.23 mmol), and HOPO (31 mg, 0.28 mmol). The reaction mixture was stirred at 40 °C for 15 min, and then P11 (26 mg, 0.11 mmol) was added. The suspension was stirred at 40 °C for 12 h then purified by HPLC (C₁₈ 150 mm×40 mm×5 μm column, Mobile Phase A: H₂O (NH₄OH—NH₄HCO₃)/Mobile Phase B: ACN, 5 to 45% of Mobile Phase B over 9 min, 100% of Mobile Phase B hold 2 min, flow rate: 60 mL/min) to provide 286 (26 mg, 50% yield) as a white solid. (LC/MS) m/z (M+H)⁺=547.3. ¹H NMR (400 MHz, (CD₃)₂SO) δ 8.33-8.16 (m, 2H), 7.91 (s, 1H), 7.35 (d, 3H), 7.14 (d, 2H), 6.79 (d, 1H), 4.60 (s, 2H), 4.50-4.37 (m, 1H), 4.35-4.27 (m, 2H), 3.89-3.76 (m, 2H), 3.53-3.44 (m, 2H), 2.97 (s, 3H), 2.87 (s, 6H), 2.80-2.70 (m, 5H), 1.35 (d, 6H).

### Example 322

### rac-(R)—N-(4-(3,3-Dimethylureido)benzyl)-1-isopropyl-5-(5-(tetrahydrofuran-2-yl)pyridin-2-yl)-4,5,6,7-tetrahydro-1H-pyrazolo[4,3-c]pyridine-3-carboxamide (322)

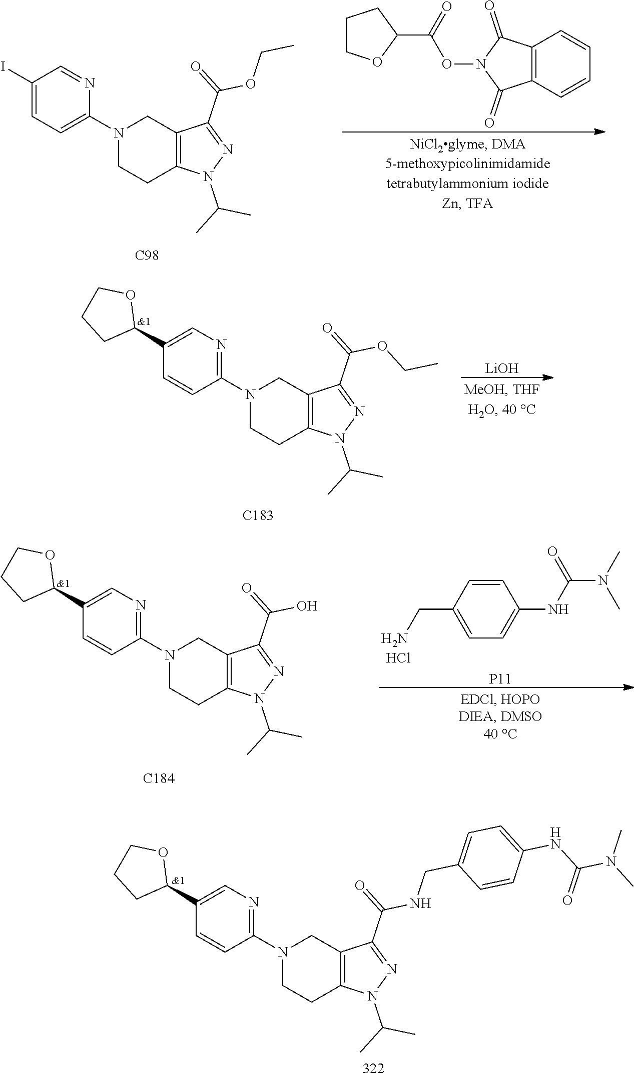

### Step 1. Preparation of rac-ethyl (R)-1-isopropyl-5-(5-(tetrahydrofuran-2-yl)pyridin-2-yl)-4,5,6,7-tetrahydro-1H-pyrazolo[4,3-c]pyridine-3-carboxylate (C183)

A reaction mixture of NiCl₂·glyme (59.9 mg, 0.273 mmol) and 5-methoxypicolinimidamide (41.2 mg, 0.273 mmol) in DMA (1.4 mL) was stirred at room temperature for 2 min before C98 (0.120 g, 0.273 mmol) and 1,3-dioxoisoindolin-2-y1 tetrahydrofuran-2-carboxylate (CAS: 1872262-69-5; 0.107 g, 0.409 mmol) were added. The reaction mixture was stirred for 2 min then tetrabutylammonium iodide (CAS: 311-28-4; 0.101 g, 0.273 mmol), preactivated Zn (0.125 g, 1.91 mmol), and TFA (15.5 mg, 0.136 mmol) were added. The suspension was stirred at room temperature for 16 h and then diluted with H₂O (30 mL) and EtOAc (20 mL). The mixture was filtered through Celite. The filtrate was concentrated in vacuo and the residue was purified by reverse phase HPLC (C₁₈ 150 mm×40 mm×5 μm column, Mobile Phase A: H₂O (NH₄OH—NH₄HCO₃)/Mobile Phase B: ACN, 39 to 59% of Mobile Phase B over 9 min, 100% of Mobile Phase B hold 2 min, flow rate: 60 mL/min) to provide C183 (25.1 mg, 26.6% yield) as a gum. (LC/MS) m/z (M+H)⁺=385.2. ¹H NMR (400 MHz, CD₃OD) δ 8.09 (d, 1H), 7.59 (dd, 1H), 6.89 (d, 1H), 4.81-4.73 (m, 1H), 4.67 (s, 2H), 4.63-4.48 (m, 1H), 4.40 (q, 2H), 4.11-4.01 (m, 1H), 3.96 (t, 2H), 3.93-3.84 (m, 1H), 2.86 (t, 2H), 2.35-2.23 (m, 1H), 2.12-1.98 (m, 2H), 1.87-1.73 (m, 1H), 1.52-1.36 (m, 9H).

### Step 2. Preparation of rac-(R)-1-isopropyl-5-(5-(tetrahydrofuran-2-yl)pyridin-2-yl)-4,5,6,7-tetrahydro-1H-pyrazolo[4,3-c]pyridine-3-carboxylic acid (C184)

To a solution of C183 (23.7 mg, 0.616 mmol) in THF (1.0 mL), MeOH (0.5 mL), and H₂O (0.5 mL) was added LiOH (7.76 mg, 0.185 mmol). The reaction mixture was stirred at 40 °C for 3 h, and then 2M HCl was added to pH-5. The acidic yellow solution was concentrated in vacuo then lyophilized to form C184 (22.0 mg, crude) as a light brown solid. The solid was used directly in the next step without further purification. (LC/MS) m/z (M+H)⁺=357.1

### Step 3. Preparation of rac-(R)—N-(4-(3,3-dimethylureido)benzyl)-1-isopropyl-5-(5-(tetrahydrofuran-2-yl)pyridin-2-yl)-4,5,6,7-tetrahydro-1H-pyrazolo[4,3-c]pyridine-3-carboxamide (322)

A reaction mixture of C184 (22.0 mg, 0.0617 mmol), HOPO (20.6 mg, 0.185 mmol), EDCI (28.4 mg, 0.148 mmol), and DIEA (39.9 mg, 0.309 mmol) in DMSO (1.0 mL) was stirred at 40 °C for 30 min. The suspension was added P11 (21.3 mg, 0.0926 mmol), and then the reaction mixture was stirred at 40 °C for 6 h. The yellow reaction mixture was purified with reverse phase HPLC (C₁₈ 150 mm×30 mm×5 μm column, Mobile Phase A: H₂O (NH₄OH—NH₄HCO₃)/Mobile Phase B: ACN, 16 to 56% of Mobile Phase B over 9 min, 100% of Mobile Phase B hold 2 min, flow rate: 30 mL/min) and lyophilized to provide 322 (18.4 mg, 55.9% yield) as a white solid. (LC/MS) m/z (M+H)⁺=532.2. ¹H NMR (400 MHz, CD₃OD) δ 8.10 (d, 1H), 7.61 (dd, 1H), 7.39-7.35 (m, 2H), 7.34-7.28 (m, 2H), 6.91 (d, 1H), 4.81-4.76 (m, 1H), 4.70 (s, 2H), 4.56-4.50 (m, 3H), 4.12-3.87 (m, 4H), 3.05-3.03 (m, 6H), 2.87 (t, 2H), 2.36-2.24 (m, 1H), 2.15-2.00 (m, 2H), 1.90-1.77 (m, 1H), 1.49 (d, 6H). The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 4.0 mL/min; backpressure: 1500 psi; column temperature: 35 °C to show a mixture of peak 1 ((SFC-MS) m/z (M+H)⁺=532.16 at retention time: 0.734 min, 49.42% ee) and peak 2 ((SFC-MS) m/z (M+H)⁺=532.17 at retention time: 1.055 min, 50.58% ee).

### Example 338

### N-(4-(3,3-Dimethylureido)benzyl)-1-isopropyl-5-(5-(oxetan-3-yl)pyridin-2-yl)-4,5,6,7-tetrahydro-1H-pyrazolo[4,3-c]pyridine-3-carboxamide (338)

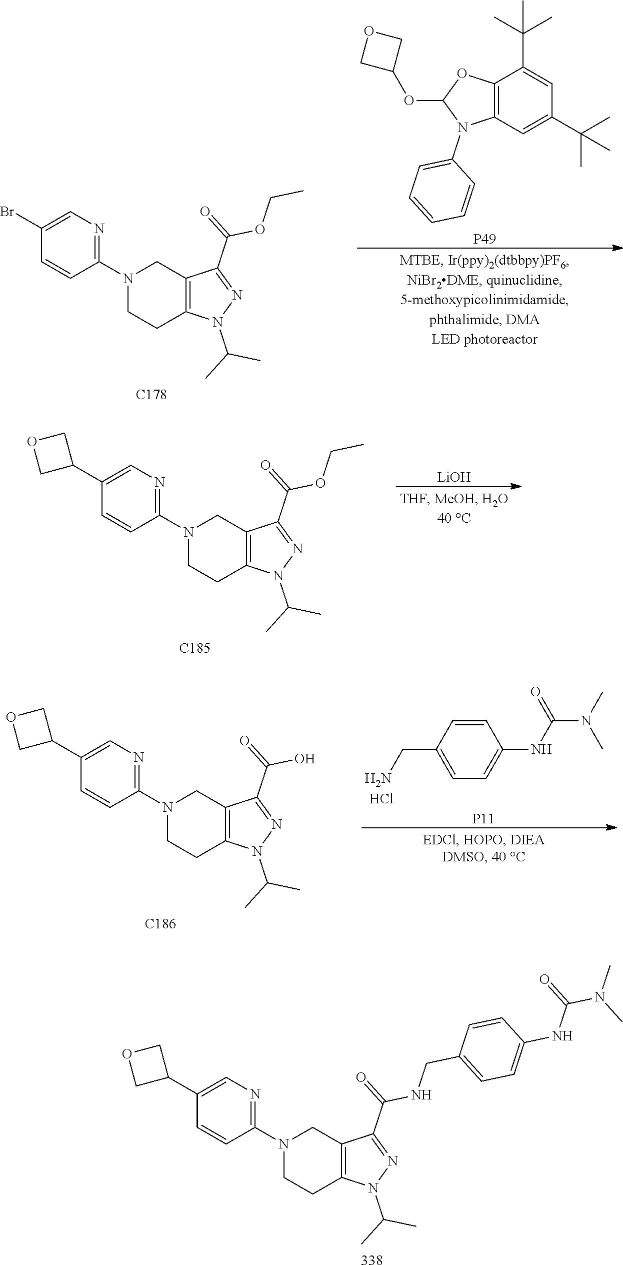

### Step 1. Preparation of ethyl 1-isopropyl-5-(5-(oxetan-3-yl)pyridin-2-yl)-4,5,6,7-tetrahydro-1H-pyrazolo[4,3-c]pyridine-3-carboxylate (C185)

In a glovebox, to C178 (0.100 g, 0.254 mmol) was added Ir(ppy)₂(dtbbpy)PF₆ (CAS: 676525-77-2; 3.49 mg, 0.00381 mmol), NiBr₂·DME (7.85 mg, 0.0254 mmol), 5-methoxypicolinimidamide (CAS: 1179532-65-0; 3.84 mg, 0.0254 mmol), phthalimide (9.35 mg, 0.0636 mmol), quinuclidine (49.5 mg, 0.445 mmol), and DMA (3 mL). A solution of P49 (194 mg, 0.509 mmol) in MTBE (3 mL) was added to the suspension via a syringe with a filter. The reaction vessel was sealed and irradiated with a photoreactor (maximum fan speed; stirred speed: 1500 rpm; 100% light intensity LED) for 16 h. The reaction mixture was concentrated in vacuo, then basified with aqueous NH₄OH and diluted with MeOH (1 mL). The solution was purified by reverse phase HPLC (C₁₈ 150 mm×40 mm×5 μm column, Mobile Phase A: H₂O (NH₄OH—NH₄HCO₃)/Mobile Phase B: ACN, 21 to 61% of Mobile Phase B over 9 min, 100% of Mobile Phase B hold 2 min, flow rate: 60 mL/min) to provide C185 (25.1 mg, 26.6% yield) as a gum. (LC/MS) m/z (M+H)⁺=371.3. ¹H NMR (400 MHz, CD₃OD) δ 8.05 (d, 1H), 7.78 (dd, 1H), 6.93 (d, 1H), 5.03 (dd, 2H), 4.71-4.62 (m, 4H), 4.60-4.46 (m, 1H), 4.38 (q, 2H), 4.23-4.11 (m, 1H), 3.94 (t, 2H), 2.84 (t, 2H), 1.45 (d, 6H), 1.41 (t, 3H).

### Step 2. Preparation of 1-isopropyl-5-(5-(oxetan-3-yl)pyridin-2-yl)-4,5,6,7-tetrahydro-1H-pyrazolo[4,3-c]pyridine-3-carboxylic acid (C₁₈₆)

To a solution of C185 (25.1 mg, 0.0678 mmol) in THF (1.0 mL), MeOH (0.5 mL), and H₂O (0.5 mL) was added LiOH (8.53 mg, 0.203 mmol). The reaction mixture was then stirred at 40 °C for 2.5 h and then concentrated in vacuo. The residue was diluted with H₂O (0.5 mL), and then the pH was adjusted with diluted formic acid in H₂O to pH=6. The resulting yellow solution was lyophilized under high vacuum to provide C186 (23.2 mg, crude) as a light brown solid. This solid was used directly in the next step without further purification. (LC/MS) m/z (M+H)⁺=343.2.

### Step 3. Preparation of N-(4-(3,3-dimethylureido)benzyl)-1-isopropyl-5-(5-(oxetan-3-yl)pyridin-2-yl)-4,5,6,7-tetrahydro-1H-pyrazolo[4,3-c]pyridine-3-carboxamide (338)

A reaction mixture of C186 (23.2 mg, 0.0678 mmol), HOPO (22.6 mg, 0.203 mmol), EDCI (31.2 mg, 0.163 mmol), and DIEA (43.8 mg, 0.339 mmol) in DMSO (2.0 mL) was stirred at 40 °C for 30 min. P11 (23.4 mg, 0.102 mmol) was added to the suspension, and then the reaction mixture was stirred at 40 °C for 8 h. The yellow reaction mixture was purified with reverse phase HPLC (C₁₈ 150 mm×30 mm×5 μm column, Mobile Phase A: H₂O (NH₄OH—NH₄HCO₃)/Mobile Phase B: ACN, 9 to 52% of Mobile Phase B over 9 min, 100% of Mobile Phase B hold 2 min, flow rate: 30 mL/min) and lyophilized to provide 338 (6.51 mg, 18.6% yield) as a white solid. (LC/MS) m/z (M+H)⁺=518.3. ¹H NMR (400 MHz, CD₃OD) δ 8.06 (d, 1H), 7.79 (dd, 1H), 7.36-7.31 (m, 2H), 7.30-7.25 (m, 2H), 6.94 (d, 1H), 5.08-5.01 (m, 2H), 4.72-4.65 (m, 4H), 4.55-4.47 (m, 3H), 4.23-4.14 (m, 1H), 3.97 (t, 2H), 3.00 (s, 6H), 2.84 (t, 2H), 1.45 (d, 6H).

### Example 395

### (S)—N-(4-Carbamoylbenzyl)-5-(5-(6,7-dihydro-5H-pyrrolo[3,4-b]pyridine-6-carbonyl)pyridin-2-yl)-1-isopropyl-7-methyl-4,5,6,7-tetrahydro-1H-pyrazolo[4,3-c]pyridine-3-carboxamide (395)

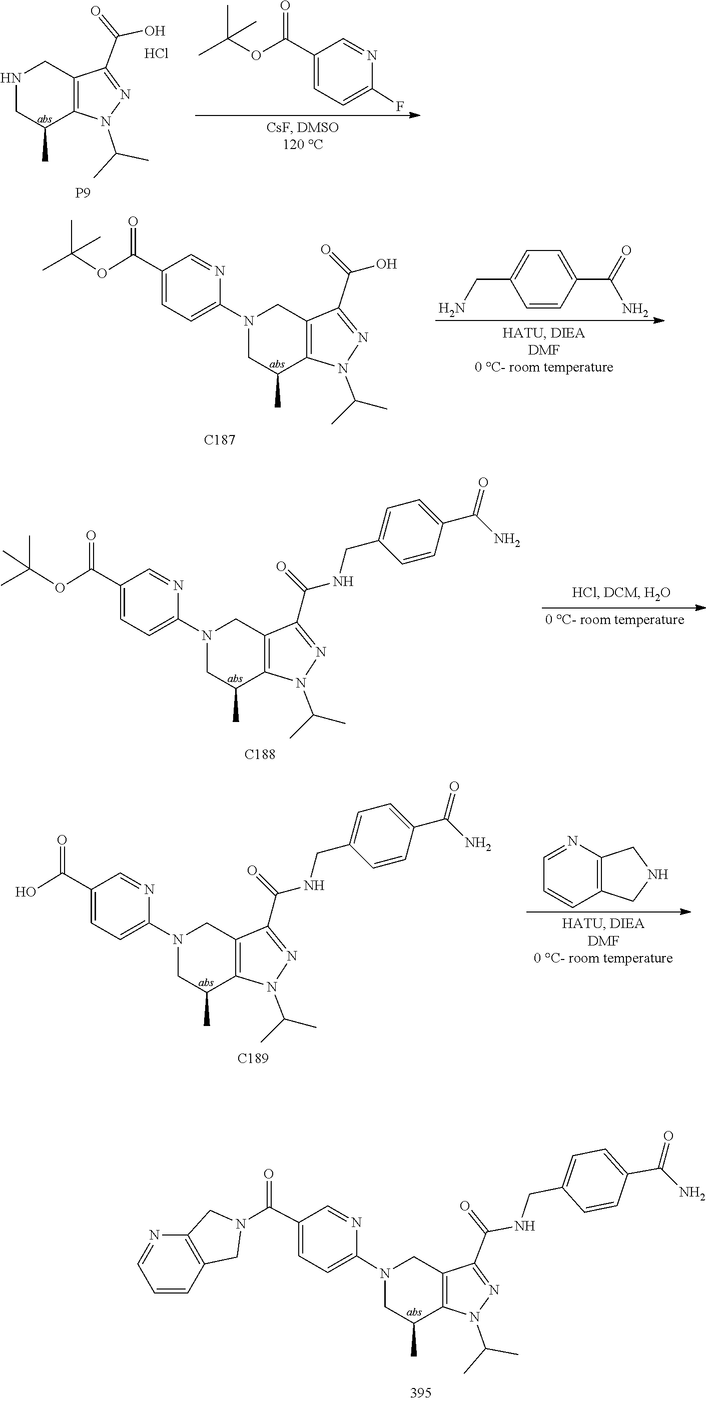

### Step 1. Preparation of (S)-5-(5-(tert-butoxycarbonyl)pyridin-2-yl)-1-isopropyl-7-methyl-4,5,6,7-tetrahydro-1H-pyrazolo[4,3-c]pyridine-3-carboxylic acid (C187)

To a reaction mixture of P9 (0.10 g, 0.45 mmol) and CsF (0.17 g, 1.1 mmol) in DMSO (4 mL) was added tert-butyl 6-fluoronicotinate (CAS: 676560-01-3, 88 mg, 0.45 mmol) and then stirred at 120 °C for 16 h. The reaction mixture was diluted with H₂O (5 mL) and then extracted with (1:10, MeOH: DCM, 3×2 mL). The combined organic layer was washed with brine (2×3 mL), dried with Na₂SO₄, filtered, and then concentrated in vacuo. The residue was purified by column chromatography (silica gel, 0-20% EtOAc: PE) to provide C187 (55 mg, 31% yield) as a yellow oil. (LCMS) m/z (M+H)⁺=401.2.

### Step 2. Preparation of tert-butyl (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinate (C188)

At 0 °C, to a solution of C187 (55 mg, 0.14 mmol) in DMF (2 mL) was added HATU (52 mg, 0.14 mmol) and then stirred for 30 min. Then DIEA (71 mg, 0.55 mmol) and P11 (26 mg, 0.14 mmol) were added. The reaction mixture was stirred at room temperature for 2 h and then poured into ice water (4 mL). The suspension was filtered, and the filter cake was collected to provide C188 (65 mg, 89% yield) as a solid. The solid was used directly in the next step without further purification. (LCMS) m/z (M+H)⁺=533.3.

### Step 3. Preparation of (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinic acid hydrochloride (C189)

At 0 °C, to a solution of C188 (0.14 g, 0.26 mmol) in DCM (2 mL) was added 2N HCl in dioxane (2 mL) and then stirred for 4 h. The reaction mixture was concentrated in vacuo to provide C189 (67 mg, crude) as a white solid. The solid was used directly in the next step without further purification. (LCMS) m/z (M+H)⁺=477.1.

### Step 4. Preparation of (S)—N-(4-carbamoylbenzyl)-5-(5-(6,7-dihydro-5H-pyrrolo[3,4-b]pyridine-6-carbonyl)pyridin-2-yl)-1-isopropyl-7-methyl-4,5,6,7-tetrahydro-1H-pyrazolo[4,3-c]pyridine-3-carboxamide (395)

To a solution of C189 (67 mg, 0.13 mmol) in DMF (2 mL) was added HATU (0.050 g, 0.13 mmol). The reaction mixture was stirred at 0 °C for 30 min, then DIEA (67 mg, 0.52 mmol) and 6,7-dihydro-5H-pyrrolo[3,4-b]pyridine (CAS: 147739-88-6; 16 mg, 0.13 mmol) were added. The suspension was stirred for 2 h at room temperature, poured into ice water (8 mL), and extracted with EtOAc (3×5 mL). The combined organic layer was washed with Na₂CO₃ (2×5 mL) and brine (10 mL), dried with Na₂SO₄, and then concentrated in vacuo. The residue was dissolved in DMF then purified by reverse phase HPLC (Boston Prime C₁₈ 150 mm×30 mm×5 μm column; Mobile Phase A: H₂O (0.05% formic acid)/Mobile Phase B: ACN, 25 to 45% of Mobile Phase B over 10 min, held at 100% of Mobile Phase B for 2 min; flow rate: 35 mL/min) and lyophilized to provide 395 (19 mg, 25% yield) as a white solid. (LCMS) m/z (M+H)⁺=579.4. ¹H NMR (400 MHz, (CD₃)₂SO) δ 8.55 (t, 1H), 8.50-8.41 (m, 2H), 7.92-7.75 (m, 5H), 7.36 (d, 2H), 7.32-7.26 (m, 2H), 6.95-6.90 (m, 1H), 5.12 (d, 1H), 4.99-4.78 (m, 4H), 4.60-4.40 (m, 4H), 4.25 (d, 1H), 3.28-3.20 (m, 2H), 1.47-1.38 (m, 6H), 1.20 (d, 3H). The chiral purity was determined using SFC analytical method on Chiralpak AS-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 3.0 mL/min; column temperature: 35 °C to provide Example 395: peak 1 ((SFC-MS) m/z (M+H)⁺=579.19 at retention time: 1.586 min, 98% ee)

### Example 469

### rac-(R)—N-(4-(3,3-Dimethylureido)benzyl)-1-isopropyl-5-(5-(2-oxopyrrolidin-3-yl)pyridin-2-yl)-4,5,6,7-tetrahydro-1H-pyrazolo[4,3-c]pyridine-3-carboxamide (469)

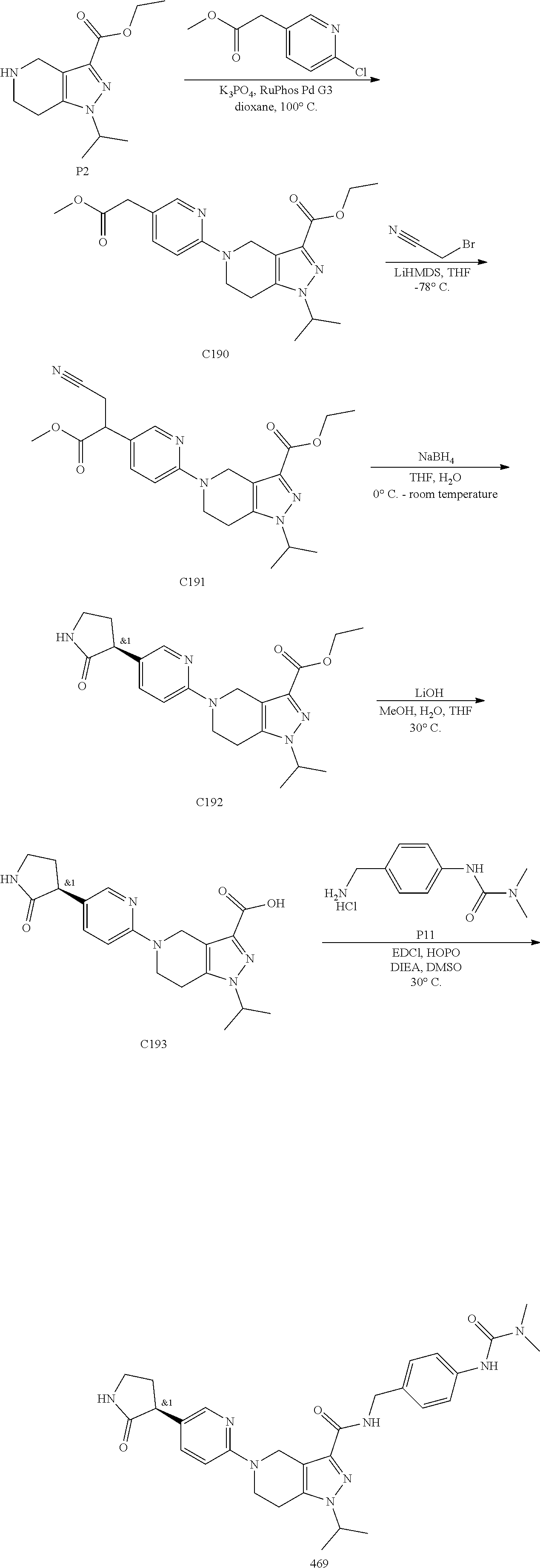

### Step 1. Preparation of ethyl 1-isopropyl-5-(5-(2-methoxy-2-oxoethyl)pyridin-2-yl)-4,5,6,7-tetrahydro-1H-pyrazolo[4,3-c]pyridine-3-carboxylate (C190)

A reaction mixture of methyl 2-(6-chloropyridin-3-yl)acetate (CAS: 717106-69-9; 1.40 g, 7.54 mmol), P2 (2.17 g, 7.92 mmol), K₃PO₄ (4.80 g, 22.6 mmol) and RuPhos Pd G3 (0.631 g, 0.754 mmol) in dioxane (50 mL) was degassed with nitrogen gas for 2 min. The reaction mixture was stirred at 100 °C for 24 h and then filtered. The filter cake was washed with EtOAc. The filtrate was purified by column chromatography (silica gel, 0-62% EtOAc: PE) to give C₁₉₀ (2.22 g, 76.2% yield) as a yellow solid. ¹H NMR (400 MHz, CDCl₃) δ 8.07 (d, 1H), 7.45 (dd, 1H), 6.72 (d, 1H), 4.62 (s, 2H), 4.54-4.38 (m, 3H), 4.08-4.03 (m, 2H), 3.68 (s, 3H), 3.50 (s, 2H), 2.81 (t, 2H), 1.52 (d, 6H), 1.42 (t, 3H).

### Step 2. Preparation of ethyl 5-(5-(3-cyano-1-methoxy-1-oxopropan-2-yl)pyridin-2-yl)-1-isopropyl-4,5,6,7-tetrahydro-1H-pyrazolo[4,3-c]pyridine-3-carboxylate (C₁₉₁)

At −78 °C under nitrogen gas, to a solution of C190 (1.72 g, 4.45 mmol) in THF (22 mL) was added LiHMDS (1.12 g, 6.68 mmol). The reaction mixture was stirred at −78 °C for 30 min under nitrogen gas, and then 2-bromoacetonitrile (CAS: 590-17-0; 0.667 g, 5.56 mmol) was added dropwise. The suspension was stirred at −78 °C for 2 h and quenched with NH₄Cl (20 mL). The reaction mixture was extracted with EtOAc (3×). The combined organic phase was washed with brine and then dried with Na₂SO₄ and filtered. The filtrate was concentrated in vacuo then purified by column chromatography (silica gel, 0-50% EtOAc: PE) to provide C191 (1.70 g, 69.6% yield) as a yellow gum. (LCMS) m/z (M+H)⁺=426.2. ¹H NMR (400 MHz, CDCl₃) δ 8.10 (d, 1H), 7.43 (dd, 1H), 6.74 (d, 1H), 4.65 (s, 2H), 4.55-4.38 (m, 3H), 4.17-4.05 (m, 2H), 3.89-3.80 (m, 1H), 3.72 (s, 3H), 2.98 (dd, 1H), 2.86-2.74 (m, 3H), 1.52 (d, 6H), 1.42 (t, 3H).

### Step 3. Preparation of rac-ethyl (R)-1-isopropyl-5-(5-(2-oxopyrrolidin-3-yl)pyridin-2-yl)-4,5,6,7-tetrahydro-1H-pyrazolo[4,3-c]pyridine-3-carboxylate (C192)

The reaction was conducted in two batches combined for the work-up and purification. At 0 °C, under nitrogen gas, to a solution of C191 (1.50 g, 3.52 mmol) in THF (20 mL) and H₂O (3 mL), NaBH₄ (0.534 g, 14.1 mmol) was slowly added to form the first batch. The reaction mixture of the first batch was stirred for 3 h at room temperature and then quenched with saturated NH₄Cl (20 mL) and filtered through Celite.

A second batch of the same reaction was conducted with C191 (0.200 g, 0.470 mmol). The two batches were combined and extracted with EtOAc (3×50 mL). The combined organic layer was washed with brine (10 mL), dried over Na₂SO₄, filtered, and concentrated in vacuo. The residue was purified by column chromatography (silica gel, 0-65% EtOAc: PE) to provide C192 (0.920 g, 58.0% yield) as a white solid. (LCMS) m/z (M+H)⁺=398.2. ¹H NMR (400 MHz, CDCl₃) δ 8.10 (d, 1H), 7.45 (dd, 1H), 6.75 (d, 1H), 4.64 (s, 2H), 4.54-4.38 (m, 3H), 4.07 (t, 2H), 3.92-3.80 (m, 2H), 3.16-3.05 (m, 1H), 2.87-2.77 (m, 3H), 2.69 (dd, 1H), 1.70-1.66 (m, 1H), 1.52 (d, 6H), 1.43 (t, 3H).

### Step 4. Preparation of rac-(R)-1-isopropyl-5-(5-(2-oxopyrrolidin-3-yl)pyridin-2-yl)-4,5,6,7-tetrahydro-1H-pyrazolo[4,3-c]pyridine-3-carboxylic acid (C193)

The same reaction was conducted in two batches and combined for the work-up. To a solution of C192 (0.13 g, 0.33 mmol) in THF (20.0 mL), H₂O (3.0 mL), and MeOH (0.5 mL) was added LiOH (69 mg, 1.6 mmol) to form the first batch. The reaction mixture of the first batch was stirred at 30 °C for 3 h, and then the second batch of the same reaction was conducted with C192 (0.020 g, 0.050 mmol). The two batches were combined then the pH was adjusted to pH-3 with 2M aqueous HCl. The aqueous phase was concentrated in vacuo to provide C193 (0.14 g, crude) as a white solid. The solid was used directly in the next step without further purification. (LCMS) m/z (M+H)⁺=370.1. ¹H NMR (400 MHz, (CD₃)₂SO) δ 12.64 (br s, 1H), 7.98 (d, 1H), 7.46 (dd, 1H), 6.81 (d, 1H), 5.00 (t, 1H), 4.56 (s, 2H), 4.50-4.37 (m, 1H), 3.80 (t, 2H), 3.49-3.38 (m, 2H), 2.92-2.78 (m, 2H), 2.77-2.66 (m, 3H), 1.30 (d, 6H).

### Step 5. Preparation of rac-(R)—N-(4-(3,3-dimethylureido)benzyl)-1-isopropyl-5-(5-(2-oxopyrrolidin-3-yl)pyridin-2-yl)-4,5,6,7-tetrahydro-1H-pyrazolo[4,3-c]pyridine-3-carboxamide (469)

The same reaction was conducted in two batches and then combined for purification. To a reaction mixture of C₁₉₃ (0.11 g, 0.30 mmol) in DMSO (3 mL) was added DIEA (0.38 g, 3.0 mmol), HOPO (99 mg, 0.89 mmol), and EDCI (0.14 g, 0.71 mmol) for the first batch. The suspension was stirred at 30 °C for 15 min, and then P11 (0.10 g, 0.45 mmol) was added to form the first batch. The reaction mixture of the first batch was stirred at 30 °C for 5 h.

The second batch of the same reaction was conducted with C193 (0.020 g, 0.054 mmol). The two batches were combined then purified reverse phase HPLC (C₁₈ 150 mm×40 mm×5 μm column, Mobile Phase A: H₂O (0.05% formic acid)/Mobile Phase B: ACN, 5 to 35% of Mobile Phase B over 9 min then held at 100% of Mobile Phase B for 2 min; flow rate=60 mL/min) and lyophilized to provide 469 (0.080 g, 41.7% yield) as a white solid. (LC/MS) m/z (M+H)⁺=545.3. ¹H NMR (400 MHz, CDCl₃) δ 8.07 (d, 1H), 7.40 (dd, 1H), 7.37-7.33 (m, 2H), 7.31-7.27 (m, 2H), 7.16 (t, 1H), 6.79 (d, 1H), 6.32 (s, 1H), 4.70 (s, 2H), 4.55 (d, 2H), 4.42-4.31 (m, 1H), 4.08 (t, 2H), 3.93-3.76 (m, 2H), 3.12-3.00 (m, 7H), 2.86-2.75 (m, 3H), 2.71-2.60 (m, 1H), 1.44 (d, 6H). The chiral purity was determined using SFC analytical method on Chiralpak AD-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 4.0 mL/min; column temperature: 35 °C to show a mixture of peak 1 ((SFC-MS) m/z (M+H)⁺=545.55 at retention time: 1.006 minutes, 49.54% ee) and peak 2 ((SFC-MS) m/z (M+H)⁺=545.55 at retention time: 1.470 min, 50.46% ee).

### Example 485

### (S)-5-(4-(Dimethylcarbamoyl)phenyl)-1-isopropyl-7-methyl-N-(4-(4-(piperidin-4-yloxy)piperidine-1-carboxamido)benzyl)-4,5,6,7-tetrahydro-1H-pyrazolo[4,3-c]pyridine-3-carboxamide hydrochloride (485)

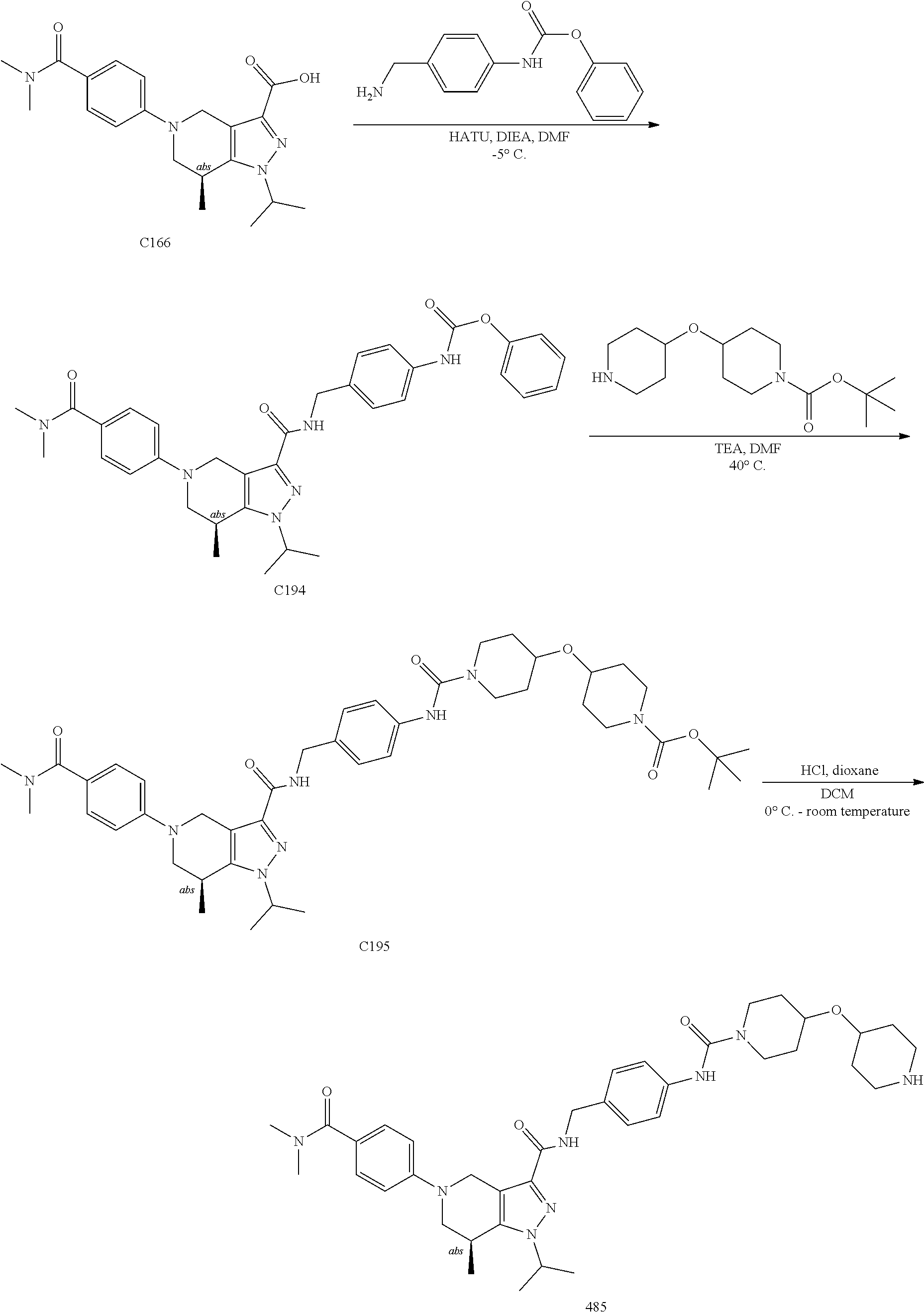

### Step 1. Preparation of phenyl (S)-(4-((5-(4-(dimethylcarbamoyl)phenyl)-1-isopropyl-7-methyl-4,5,6,7-tetrahydro-1H-pyrazolo[4,3-c]pyridine-3-carboxamido)methyl)phenyl)carbamate (C194)

At −5 °C, to a reaction mixture of C166 (1.20 g, 3.24 mmol) and phenyl (4-(aminomethyl)phenyl)carbamate (CAS: 1019455-66-3; 0.957 g, 3.43 mmol) in DMF (16 mL) was added HATU (1.29 g, 3.40 mmol). The reaction mixture was stirred at −5 °C for 10 min, and then DIEA (1.67 g, 13.0 mmol) was added. The suspension was stirred at −5 °C for 1 h then was poured into ice water and filtered. The filter cake was washed with H₂O (3×15 mL) and lyophilized to provide C194 (1.73 g, 90.0% yield) as a white solid. (LC/MS) m/z (M+H)⁺=595.8. ¹H NMR (400 MHz, (CD₃)₂SO) δ 10.20-10.15 (m, 1H), 8.45-8.37 (m, 1H), 7.47-7.37 (m, 4H), 7.36-7.17 (m, 7H), 6.96 (d, 2H), 4.58-4.50 (m, 2H), 4.37 (d, 2H), 4.10 (d, 1H), 3.85-3.77 (m, 1H), 3.22-3.17 (m, 2H), 2.95 (s, 6H), 1.42 (dd, 6H), 1.28 (d, 3H).

### Step 2. Preparation of tert-butyl (S)-4-((1-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)phenyl)carbamoyl)piperidin-4-yl)oxy)piperidine-1-carboxylate (C₁₉₅)

To a solution of C194 (0.300 g, 0.504 mmol) in DMF (3 mL) were added TEA (0.204 g, 2.02 mmol) and tert-butyl 4-(piperidin-4-yloxy)piperidine-1-carboxylate (CAS: 845305-83-1; 0.143 g, 0.504 mmol). The reaction mixture was stirred at 40 °C for 2 h then poured into H₂O and filtered. The filter cake was washed with H₂O (3×15 mL) and lyophilized to provide C195 (0.360 g, 90.9% yield) as a white solid. (LC/MS) m/z (M+H)⁺=785.5.

### Step 3. Preparation of (S)-5-(4-(dimethylcarbamoyl)phenyl)-1-isopropyl-7-methyl-N-(4-(4-(piperidin-4-yloxy)piperidine-1-carboxamido)benzyl)-4,5,6,7-tetrahydro-1H-pyrazolo[4,3-c]pyridine-3-carboxamide hydrochloride (485)

At 0 °C, to a solution of C195 (0.361 g, 0.460 mmol) in DCM (5 mL) was added 2M HCl in dioxane (15 mL). The reaction mixture was stirred at room temperature for 2 h then concentrated in vacuo. The residue was diluted with H₂O then purified by reverse phase HPLC (Boston Green ODS 150 mm×30 mm×5 μm column; Mobile phase: A: H₂O (0.05% aqueous HCl)/Mobile Phase B: ACN; 15-55% of Mobile Phase B over 9.0 min; flow rate: 30 mL/min) and lyophilized to provide 485 (18.7 mg, 37.3% yield) as a yellow solid. (LC/MS) m/z (M+H)⁺=685.5. ¹H NMR (400 MHz, (CD₃)₂SO) δ 8.70-8.50 (m, 2H), 8.42-8.34 (m, 1H), 8.24 (t, 1H), 7.31-7.19 (m, 4H), 7.12-7.05 (m, 2H), 6.89-6.83 (m, 2H), 4.50-4.38 (m, 2H), 4.31-4.19 (m, 2H), 4.00 (d, 1H), 3.76-3.59 (m, 4H), 3.58-3.50 (m, 1H), 3.21-2.93 (m, 6H), 2.92-2.80 (m, 8H), 1.86-1.77 (m, 2H), 1.74-1.66 (m, 2H), 1.60-1.48 (m, 2H), 1.37-1.25 (m, 7H), 1.18 (d, 3H).

### Example 498

### (S)-2-(3-((4-(3,3-Dimethylureido)benzyl)carbamoyl)-1-isopropyl-7-methyl-1,4,6,7-tetrahydro-5H-pyrazolo[4,3-c]pyridin-5-yl)pyrimidine-5-carboxylic acid (498)

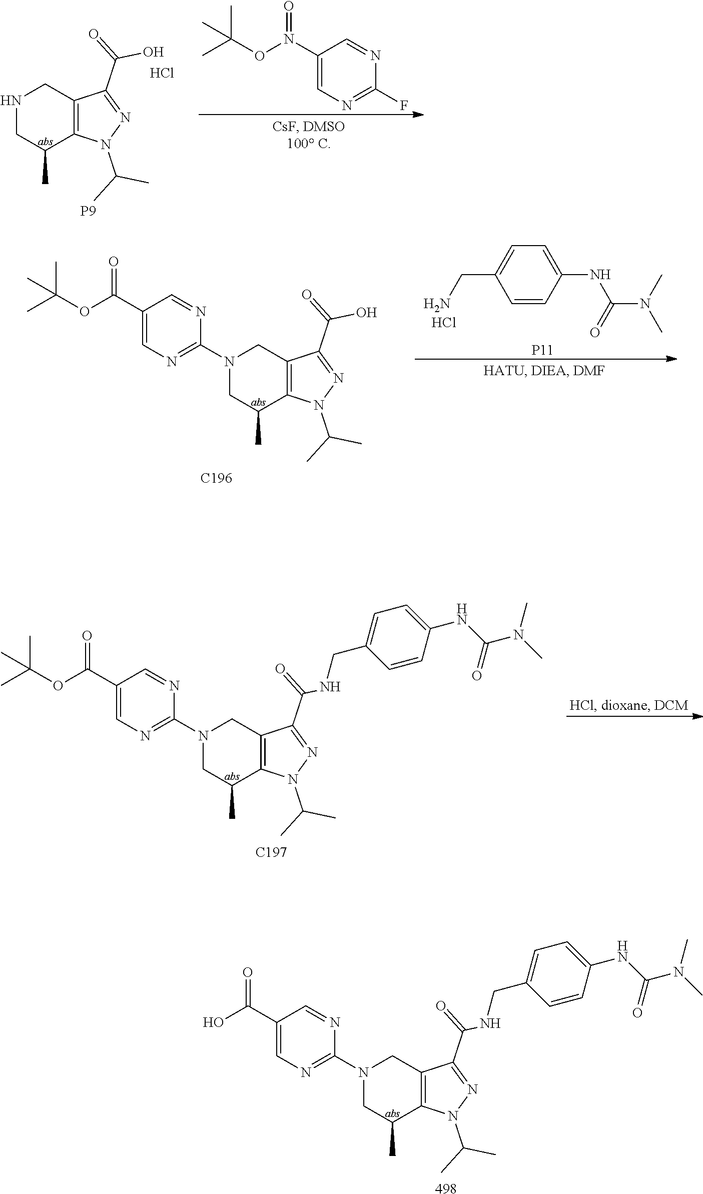

### Step 1. Preparation of (S)-5-(5-(tert-butoxycarbonyl)pyrimidin-2-yl)-1-isopropyl-7-methyl-4,5,6,7-tetrahydro-1H-pyrazolo[4,3-c]pyridine-3-carboxylic acid (C196)

To a solution of P9 (0.400 g, 1.54 mmol) and CsF (585 mg, 3.85 mmol) in DMSO (15 mL) was added tert-butyl 2-chloropyrimidine-5-carboxylate (CAS: 1864223-07-3; 331 mg, 1.54 mmol). The reaction mixture was stirred at 100 °C for 2 h and then diluted with H₂O (15 mL). The suspension was extracted with EtOAc (3×15 mL). The combined organic layer was washed with brine (3×40 mL) and then dried with Na₂SO₄ and concentrated in vacuo to provide C196 (0.610 g, crude) as a white solid. The solid was used directly in the next step without further purification. (LC/MS) m/z (M+H)⁺=402.2.

### Step 2. Preparation of tert-butyl (S)-2-(3-((4-(3,3-dimethylureido)benzyl)carbamoyl)-1-isopropyl-7-methyl-1,4,6,7-tetrahydro-5H-pyrazolo[4,3-c]pyridin-5-yl)pyrimidine-5-carboxylate (C197)

To a solution of C196 (0.610 g, 1.52 mmol) and P11 (349 mg, 1.52 mmol) in DMF (15 mL) was added HATU (607 mg, 1.60 mmol) and DIEA (786 mg, 6.08 mmol). The reaction mixture was stirred at room temperature for 30 min then was diluted with H₂O (20 mL) and extracted with EtOAc (3×20 mL). The combined organic layers were washed with brine (3×60 mL), dried with Na₂SO₄ and concentrated in vacuo. The resulting residue was purified by column chromatography (silica gel, 0-70% EtOAc:hexane) then lyophilized to provide C197 (0.780 g, 89.0% yield) as a solid. ¹H NMR (400 MHz, (CD₃)₂SO) δ 8.76 (s, 2H), 8.36 (t, 1H), 8.22 (s, 1H), 7.44-7.32 (m, 2H), 7.23-7.12 (m, 2H), 5.54 (d, 1H), 4.58-4.46 (m, 1H), 4.42-4.25 (m, 3H), 3.47-3.38 (m, 3H), 2.95-2.88 (m, 6H), 1.51 (s, 9H), 1.40 (dd, 6H), 1.10 (d, 3H).

### Step 3. Preparation of (S)-2-(3-((4-(3,3-dimethylureido)benzyl)carbamoyl)-1-isopropyl-7-methyl-1,4,6,7-tetrahydro-5H-pyrazolo[4,3-c]pyridin-5-yl)pyrimidine-5-carboxylic acid (498)

To a solution of C197 (0.780 g, 1.35 mmol) in DCM (3 mL) was added 2 M HCl in dioxane (20 mL). The reaction mixture was then stirred at room temperature for 5 h then concentrated in vacuo and lyophilized to provide 498 (0.701 g, crude) as a solid. The solid was used directly in the next step without further purification. (LC/MS) m/z (M+H)⁺=521.3. ¹H NMR (400 MHz, (CD₃)₂SO) 6 8.80 (s, 2H), 8.36 (t, 1H), 8.24 (s, 1H), 7.42-7.36 (m, 2H), 7.20-7.14 (m, 2H), 5.54 (d, 1H), 4.81 (d, 1H), 4.57-4.46 (m, 1H), 4.40-4.27 (m, 3H), 3.49-3.41 (m, 1H), 3.32-3.22 (m, 1H), 2.94-2.86 (m, 6H), 1.41 (dd, 6H), 1.13 (d, 3H). The chiral purity was determined using SFC analytical method on Chiralcel OJ-3 50 mm×4.6 mm×3 μm column; Mobile phase A: CO₂; Mobile phase B: EtOH with 0.2% of NH₃; gradient: 5-40% of Mobile Phase B over 1.5 min then held at 40% of Mobile Phase B for 1.0 min then 5% of Mobile Phase B for 0.5 min; flow rate: 3.0 mL/min; backpressure: 1500 psi; column temperature: 35 °C to provide Example 498: peak 1 ((SFC-MS) m/z (M+H)⁺=521.48 at retention time: 1.543 min, 100% ee).

### Example 515

### (S)-1-Isopropyl-7-methyl-5-(5-(1-methyl-1H-imidazol-2-yl)pyrimidin-2-yl)-N-(4-(3-methylureido)benzyl)-4,5,6,7-tetrahydro-1H-pyrazolo[4,3-c]pyridine-3-carboxamide (515)

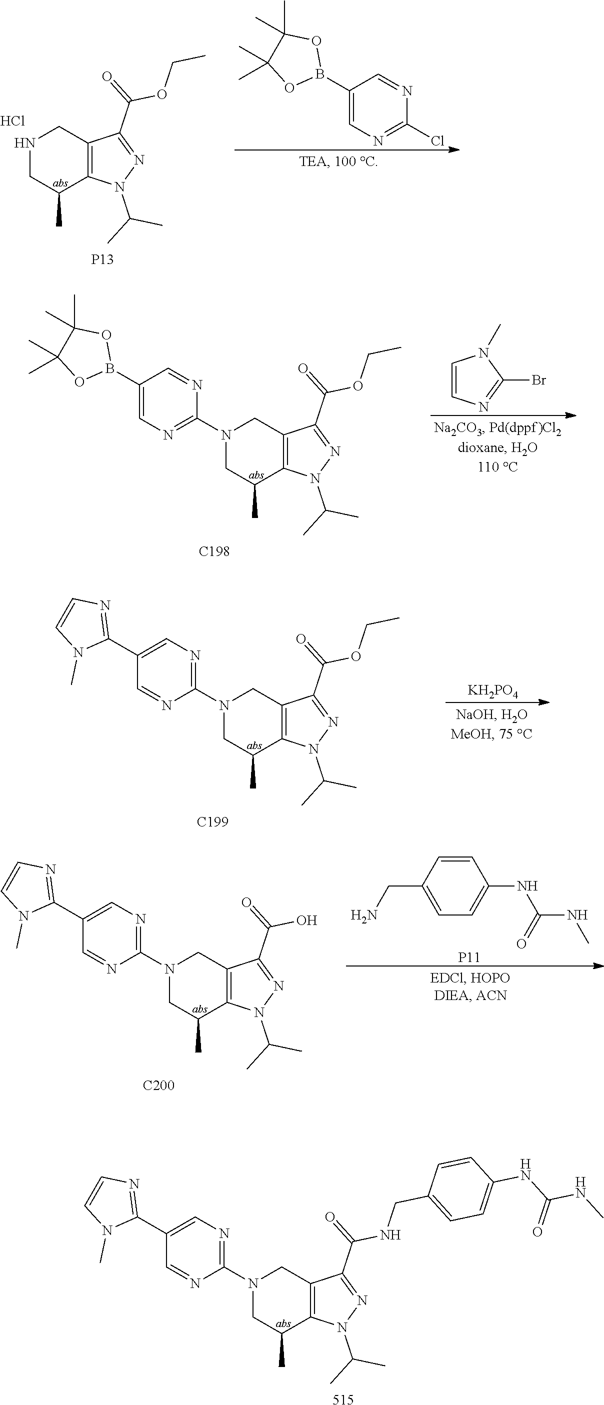

### Step 1. Preparation of ethyl (S)-1-isopropyl-7-methyl-5-(5-(4,4,5,5-tetramethyl-1,3,2-dioxaborolan-2-yl)pyrimidin-2-yl)-4,5,6,7-tetrahydro-1H-pyrazolo[4,3-c]pyridine-3-carboxylate (C198)

To a reaction mixture of P13 (0.598 g, 2.08 mmol) and TEA (0.463 g, 4.57 mmol) in dioxane (5.0 mL) was added 2-chloro-5-(4,4,5,5-tetramethyl-1,3,2-dioxaborolan-2-yl)pyrimidine (CAS: 1003845-08-6; 0.500 g, 2.08 mmol). The suspension was stirred at 100 °C for 2 h and then filtered. The filter cake was rinsed with dioxane (2.5 mL). The filtrate was diluted with saturated NaHCO₃ and extracted with EtOAc (2×25 mL). The combined organic layer was washed with brine (25 mL) then dried with Na₂SO₄ and concentrated in vacuo to give C₁₉₈ (0.865 g, crude), which was used directly in the next step without further purification. (LC/MS) m/z (M+H)⁺=456.4.

### Step 2. Preparation of ethyl (S)-1-isopropyl-7-methyl-5-(5-(1-methyl-1H-imidazol-2-yl)pyrimidin-2-yl)-4,5,6,7-tetrahydro-1H-pyrazolo[4,3-c]pyridine-3-carboxylate (C199)

In a 2-neck round bottom flask was attached a reflux condenser, to a reaction mixture of C198 (0.865 g, 1.90 mmol) in dioxane (17.6 mL) and H₂O (2.3 mL) was added Na₂CO₃ (0.604 g, 5.70 mmol) and 2-bromo-N-methylimidazole (CAS: 16681-59-7; 0.352 g, 2.18 mmol), reaction was purged with nitrogen gas for a few minutes. The reaction mixture was added Pd(dppf)Cl₂ (0.155 g, 0.190 mmol) and degassed with nitrogen gas. The suspension was stirred at 110 °C for −24 h and filtered over celite. The filter cake was washed with EtOAc (40 mL). The filtrate was concentrated in vacuo then purified by column chromatography (silica gel, 0-10% MeOH: DCM) then dried further over high vacuum to provide C199 (0.779 g, >95.0% yield). (LC/MS) m/z (M+H)⁺=410.3.

### Step 3. Preparation of (S)-1-isopropyl-7-methyl-5-(5-(1-methyl-1H-imidazol-2-yl)pyrimidin-2-yl)-4,5,6,7-tetrahydro-1H-pyrazolo[4,3-c]pyridine-3-carboxylic acid (C200)

To a stirred solution of C199 (0.779 g, 1.90 mmol) in MeOH (2.1 mL) was added 1 M NaOH in H₂O (4.2 mL). The reaction mixture was stirred at 75 °C for 20 h before KH₂PO₄ (0.828 g, 6.09 mmol) was added and then cooled to room temperature. The suspension was concentrated in vacuo to remove the MeOH. Brine (10 mL) was added to the aqueous layer extracted with DCM (2×20 mL). The combined organic layer was dried with Na₂SO₄ and then concentrated in vacuo to provide C200 (0.419 g, 57.7% yield) as a brown oil. (LC/MS) m/z (M+H)⁺=382.3.

### Step 4. Preparation of (S)-1-isopropyl-7-methyl-5-(5-(1-methyl-1H-imidazol-2-yl)pyrimidin-2-yl)-N-(4-(3-methylureido)benzyl)-4,5,6,7-tetrahydro-1H-pyrazolo[4,3-c]pyridine-3-carboxamide (515)

To a reaction mixture of P11 (51.7 mg, 0.288 mmol), HOPO (35.0 mg, 0.315 mmol), EDCI (75.4 mg, 0.393 mmol), C200 (0.100 g, 0.262 mmol) in ACN (1.3 mL) was added DIEA (0.102 g, 0.786 mmol) then stirred at room temperature for -24 h. The suspension was filtered. The filter cake and filtrate were combined then concentrated in vacuo. The residue was purified reverse phase HPLC (Sunfire C₁₈ 100 mm×19 mm×5 μm column, Mobile Phase A: H₂O (0.05% TFA)/Mobile Phase B: ACN (0.05% TFA), 70 to 80% of Mobile Phase B over 10 min, flow rate=25 mL/min) and lyophilized to provide 515 (36.4 mg, 25.6% yield). (LC/MS) m/z (M+H)⁺=543.5. ¹H NMR (600 MHz, (CD₃)₂SO) δ 8.77 (s, 1H), 8.44 (s, 1H), 8.38 (t, 1H), 7.81-7.77 (m, 2H), 7.34-7.30 (m, 2H), 7.19-7.15 (m, 2H), 5.97-5.93 (m, 1H), 5.55 (d, 1H), 4.82 (d, 1H), 4.57-4.50 (m, 1H), 4.41-4.28 (m, 3H), 3.87-3.85 (m, 3H), 3.50-3.47 (m, 2H), 3.34-3.28 (m, 1H), 2.62 (d, 3H), 1.43 (dd, 6H), 1.16 (d, 3H).

### Example 523

### (4RS,7S)-5-(5-(Dimethylcarbamoyl)pyridin-2-yl)-N-(4-(3,3-dimethylureido)benzyl)-4-hydroxy-1-isopropyl-7-methyl-4,5,6,7-tetrahydro-1H-pyrazolo[4,3-c]pyridine-3-carboxamide (523)

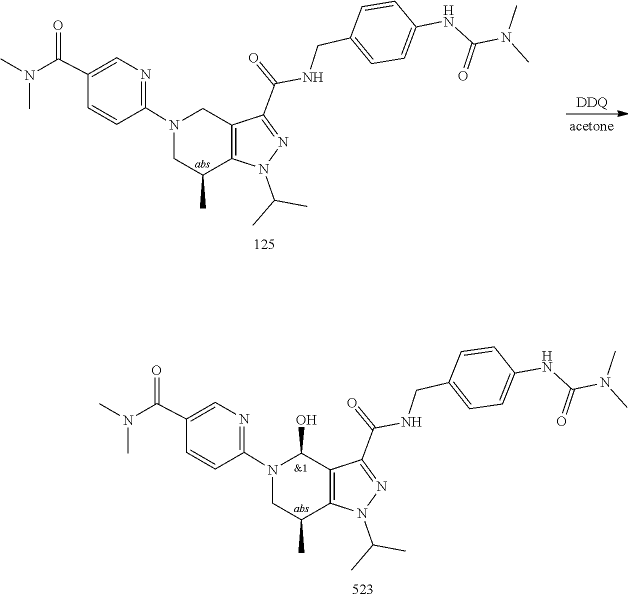

### Step 1. Preparation of (4RS,7S)-5-(5-(dimethylcarbamoyl)pyridin-2-yl)-N-(4-(3,3-dimethylureido)benzyl)-4-hydroxy-1-isopropyl-7-methyl-4,5,6,7-tetrahydro-1H-pyrazolo[4,3-c]pyridine-3-carboxamide (523)

To a reaction mixture of 125 (0.21 g, 0.38 mmol) in acetone (1.9 mL) was added DDQ (90 mg, 0.40 mmol). The suspension was stirred at room temperature under air for a few minutes and then diluted with saturated NaHCO₃ (40 mL). The mixture was extracted with DCM (40 mL), and then the organic layer was concentrated in vacuo. The residue was purified with SFC (Chiral Technologies OJ-H 250 mm×30 mm×5 μm column, Mobile Phase A: 80% CO₂/Mobile Phase B: 20% MeOH; flow rate: 80 mL/min; backpressure: 100 bar; column temperature: 40 °C) to provide 523 (0.090 g, 42% yield). ¹H NMR (600 MHz, (CD₃)₂SO) δ 8.61 (t, 1H), 8.28-8.22 (m, 2H), 7.71-7.63 (m, 1H), 7.44-7.38 (m, 2H), 7.23-7.16 (m, 2H), 7.10-7.01 (m, 1H), 6.62 (s, 1H), 5.98-5.95 (m, 1H), 4.69-4.64 (m, 1H), 4.58-4.51 (m, 1H), 4.46-4.34 (m, 2H), 3.49-3.43 (m, 1H), 3.22-3.19 (m, 1H), 3.03-2.95 (m, 6H), 2.92 (s, 6H), 1.50-1.34 (m, 6H), 1.14-1.05 (m, 3H).

### Example 524

### (4RS,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (524)

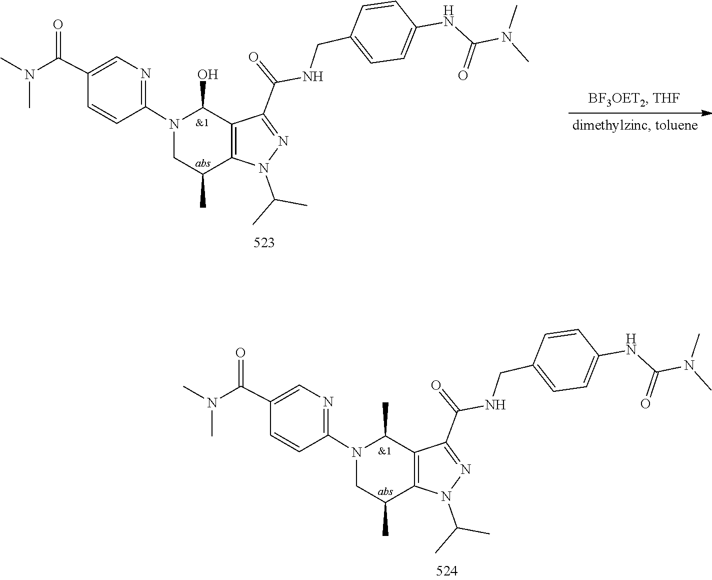

### Step 1. Preparation of (4RS,7S)-5-(5-(dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (524)

To a reaction mixture of 523 (0.100 g, 0.178 mmol) in THF (0.89 mL) was added BF₃OEt₂ (50.4 mg, 0.355 mmol). The suspension was stirred at room temperature for 15 min before 2M dimethylzinc in toluene (0.18 mL) was added. The reaction mixture was stirred at room temperature for ~72 h then quenched with H₂O. The suspension was extracted with DCM (3×). The combined organic layer was concentrated in vacuo then purified by reverse phase HPLC (Sunfire C₁₈ 100 mm×19 mm×5 μm column, Mobile Phase A: H₂O (0.05% TFA)/Mobile Phase B: ACN (0.05% TFA), 65 to 85% of Mobile Phase B over 10 min, flow rate=25 mL/min) and lyophilized to provide 524 (0.100 g, 100% yield). LC/MS m/z (M+H)⁺=561.4. ¹H NMR (600 MHz, (CD₃)₂SO) δ 8.34 (t, 1H), 8.24-8.19 (m, 2H), 7.64 (d, 1H), 7.41-7.36 (m, 2H), 7.20-7.15 (m, 2H), 6.93 (d, 1H), 5.73 (s, 1H), 4.52-4.46 (m, 2H), 4.43-4.37 (m, 1H), 4.34-4.27 (m, 1H), 3.42 (d, 1H), 3.23-3.16 (m, 1H), 2.98 (s, 6H), 2.90 (s, 6H), 1.43-1.36 (m, 9H), 1.13 (d, 3H).

### Example 529

### rel-(4S*,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (C201) and (4R,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (529)

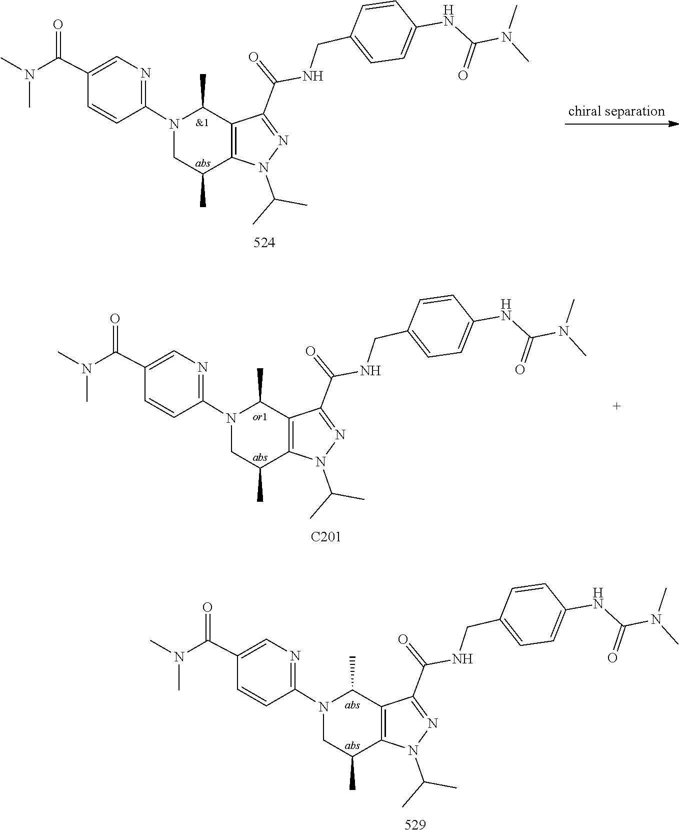

### Step 1. Preparation of rel-(4S*,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (C201) and (4R,7S)-5-(5-(Dimethylcarbamoyl)pyridin-2-yl)-N-(4-(3,3-dimethylureido)benzyl)-1-isopropyl-4,7-dimethyl-4,5,6,7-tetrahydro-1H-pyrazolo[4,3-c]pyridine-3-carboxamide (529)

Example 524 (0.100 g, 0.178 mmol) was purified with SFC [Column: Phenomenex Lux Cellulose 1, 250 mm×21 mm×5 μm; Mobile phase: 30% MeOH (0.2% NH₄OH)/70% ACN; flow rate=75 mL/min, gradient time=20 min, backpressure=120 bar] to provide the minor first eluting isomer as Example C201 (0.30 mg, 3.0% yield) and the second eluting isomer as Example 529 (38 mg, 38% yield). LC/MS m/z (M+H)⁺=561.2. ¹H NMR (600 MHz, (CD₃)₂SO) δ 8.29 (t, 1H), 8.21-8.17 (m, 2H), 7.57 (dd, 1H), 7.37-7.31 (m, 2H), 7.18-7.10 (m, 2H), 6.83 (d, 1H), 5.70 (s, 1H), 4.50-4.40 (m, 2H), 4.39-4.30 (m, 1H), 4.29-4.20 (m, 1H), 3.31-3.27 (m, 1H), 3.18-3.08 (m, 1H), 2.94 (s, 6H), 2.86 (s, 6H), 1.39-1.27 (m, 9H), 1.08 (d, 3H). The chiral purity was determined using SFC analytical method on Lux Cellulose 1, 100 mm×4.6 mm×5 μm column; Mobile phase A: MeOH (0.2% NH₄OH); Mobile phase B: ACN; isocratic gradient: 60% of Mobile Phase B over 5 min; flow rate: 5.0 mL/min; backpressure: 120 bar to provide C201: peak 1 ((SFC-MS) m/z (M+H)⁺=561.2 at retention time: 3.02 min) and Example 529: peak 2 ((SFC-MS) m/z (M+H)⁺=561.2 at retention time: 3.28 min, 99% ee).

### Example 530

### (S)—N-(4-(3,3-Dimethylureido)benzyl)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxamide (530)

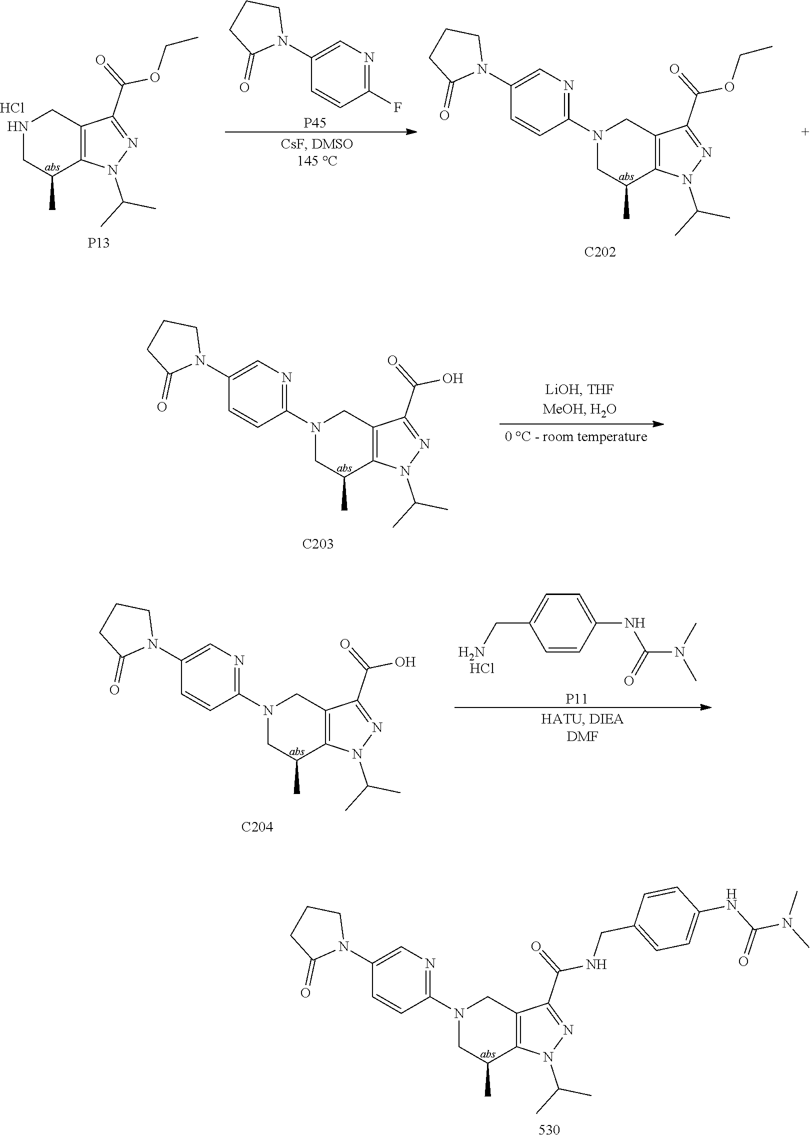

### Step 1. Preparation of ethyl (S)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxylate (C202) and (S)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxylic acid (C203)

To a solution of P45 (0.169 g, 0.938 mmol) and P13 (0.270 g, 0.938 mmol) in DMSO (3 mL) was added CsF (0.855 g, 5.63 mmol). The reaction mixture was stirred at 145 °C for 48 h then diluted with H₂O (10 mL) and acidified with aqueous 1 M HCl to pH=4. The suspension was extracted with EtOAc (4×30 mL). The combined organic layer was washed with brine (20 mL) then dried with Na₂SO₄ and concentrated in vacuo to provide (61: 22 according to LCMS) C203 (LC/MS) m/z (M+H)⁺=384.2: C202 (LC/MS) m/z (M+H)⁺=412.3 (0.290 g, crude) as a yellow gum. The gum was used directly in the next step without further purification.

### Step 2. Preparation of (S)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxylic acid (C204)

At 0 °C, to the solution of a mixture of C202 and C203 (0.29 g, 0.17 mmol) in MeOH (2 mL) and THF (2 mL) were added LiOH (15 mg, 0.35 mmol) in H₂O (1 mL). The reaction mixture was stirred at room temperature for 16 h then concentrated in vacuo and diluted with H₂O (10 mL). The suspension was extracted with EtOAc (2×10 mL). The aqueous layer was acidified with 1 M aqueous HCl to pH=3. The acidic aqueous phase was extracted with (1:10) (MeOH: DCM) (2×20 mL). The combined organic layer was washed with brine (2×20 mL) then dried with Na₂SO₄ and concentrated in vacuo to provide C204 (0.11 g, crude) as a yellow gum. The gum was used directly in the next step without further purification. (LC/MS) m/z (M+H)⁺=384.2

### Step 3. Preparation of (S)—N-(4-(3,3-dimethylureido)benzyl)-1-isopropyl-7-methyl-5-(5-(2-oxopyrrolidin-1-yl)pyridin-2-yl)-4,5,6,7-tetrahydro-1H-pyrazolo[4,3-c]pyridine-3-carboxamide (530)

To a solution of C204 (0.055 g, 0.14 mmol) and P11 (0.033 g, 0.14 mmol) in DMF (1 mL) was added DIEA (0.074 g, 0.57 mmol) and HATU (0.057 g, 0.15 mmol). The reaction was stirred at room temperature for 16 h then was filtered. The filtrate was purified by reverse phase HPLC (Boston Prime C₁₈ 150 mm×30 mm×5 μm column, Mobile Phase A: H₂O (0.05% TFA)/Mobile Phase B: ACN, 23 to 53% of Mobile Phase B over 12 min, 100% of Mobile Phase B hold 3 min, flow rate=35 mL/min) and lyophilized to provide 530 (19 mg, 24% yield) as a white solid. (LCMS) m/z (M+H)⁺=559.2. ¹H NMR (400 MHz, (CD₃)₂SO) δ 8.33 (t, 1H), 8.29 (d, 1H), 8.23 (s, 1H), 7.88 (dd, 1H), 7.43-7.35 (m, 2H), 7.21-7.15 (m, 2H), 6.90 (d, 1H), 4.95 (d, 1H), 4.59-4.48 (m, 1H), 4.40-4.28 (m, 3H), 4.17 (d, 1H), 3.78 (t, 2H), 3.32-3.18 (m, 2H), 2.91 (s, 6H), 2.44 (t, 2H), 2.12-2.00 (m, 2H), 1.47-1.38 (m, 6H), 1.21 (d, 3H). The chiral purity was determined using SFC analytical method on Chiralpak AS-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 3.0 mL/min; column temperature: 35 °C to provide Example 530: peak 1 ((SFC-MS) m/z (M+H)⁺=559.57 at retention time: 1.165 minutes, 99.53% ee)

### Example 537

### rac-(R)-7-(4-(Dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (537)

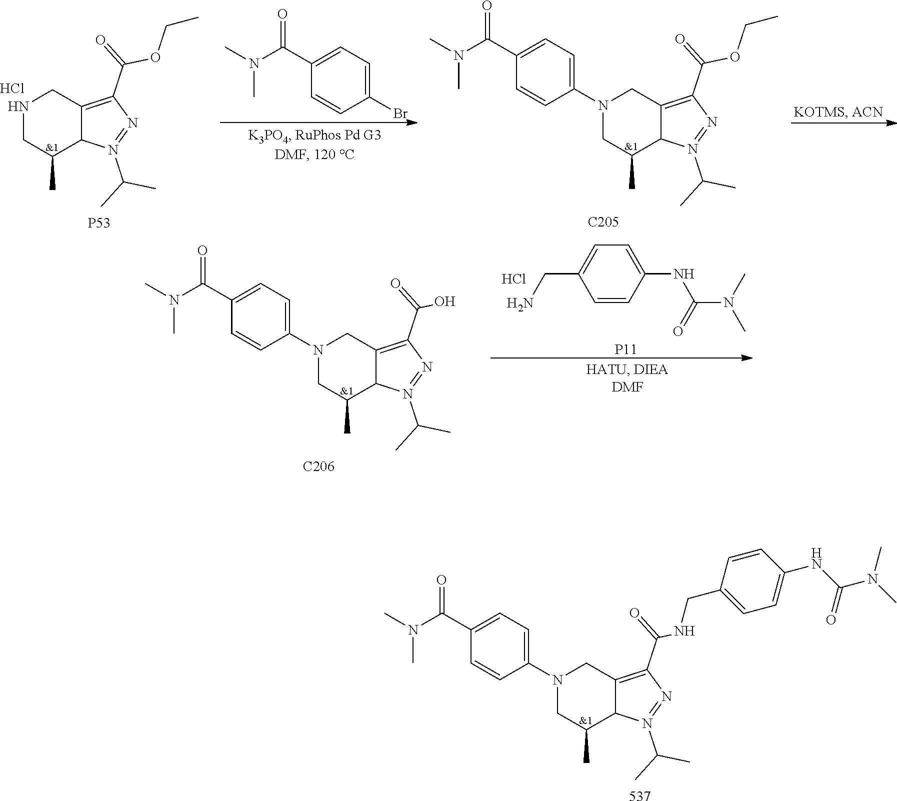

### Step 1. Preparation of rac-ethyl (R)-7-(4-(dimethylcarbamoyl)phenyl)-3-isopropyl-5-methyl-5,6,7,8-tetrahydroimidazo[1,5-a]pyrazine-1-carboxylate (C205)

To a reaction mixture of P53 (0.400 g, 1.39 mmol) in DMF (10 mL) was added 4-bromo-N,N-dimethylbenzamide (CAS: 18469-37-9; 0.396 g, 1.74 mmol), K₃PO₄ (0.885 g, 4.17 mmol) and RuPhos Pd G3 (0.119 g, 0.142 mmol). The suspension was stirred at 120 °C under nitrogen gas for 20 h. The reaction mixture was dissolved in MeOH and filtered. The filtrate was poured into H₂O (30 mL) and extracted with EtOAc (5×30 mL). The combined organic layers were washed with brine (3×100 mL), dried with Na₂SO₄, filtered and the filtrate was concentrated in vacuo to give a brown gum. The gum was purified by column chromatography (silica gel, 0-4% MeOH: DCM) to provide C₂₀₅ (0.360 g, 65.0% yield) as yellow gum. (LC/MS) m/z (M+H)⁺=399.2. ¹H NMR (400 MHz, (CD₃)₂SO) δ 7.80-7.74 (m, 1H), 7.55-7.44 (m, 1H), 7.42-7.33 (m, 2H), 7.04-6.97 (m, 2H), 4.89 (d, 1H), 4.64-4.59 (m, 1H), 4.34 (d, 1H), 4.24 (q, 2H), 3.15-3.04 (m, 1H), 2.97 (s, 6H), 1.42 (d, 3H), 1.34-1.26 (m, 6H), 1.25-1.20 (m, 3H).

### Step 2. Preparation of rac-(R)-7-(4-(dimethylcarbamoyl)phenyl)-3-isopropyl-5-methyl-5,6,7,8-tetrahydroimidazo[1,5-a]pyrazine-1-carboxylic acid (C₂₀₆)

Under nitrogen gas, to a reaction mixture of C205 (0.330 g, 0.828 mmol) in ACN (4 mL), was added KOTMS (0.319 g, 2.48 mmol). The suspension was stirred at room temperature for 2 h then acidified with 2M HCl in dioxane (0.7 mL) and concentrated in vacuo. The residue was diluted with brine (10 mL) and extracted with DCM (3×10 mL). The combined organic layers were dried over Na₂SO₄, filtered and the filtrate was concentrated in vacuo to provide C206 (0.280 g, 91.3% yield) as a yellow gum. (LC/MS) m/z (M+H)⁺=371.2.

### Step 3. Preparation of rac-(R)-7-(4-(dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (537)

To a solution of C206 (0.280 g, 0.756 mmol) and P11 (0.139 g, 0.605 mmol) in DMF (4 mL) was added DIEA (0.391 g, 3.02 mmol) and HATU (0.244 g, 0.642 mmol). The reaction was stirred at room temperature for 1 h then diluted with DMF and ACN. The solution was purified by reverse phase HPLC (C₁₈ 150 mm×30 mm×5 μm column, Mobile Phase A: H₂O (0.05% formic acid)/Mobile Phase B: ACN (0.05% formic acid), 24 to 44% of Mobile Phase B over 10 min, 100% of Mobile Phase B hold 2.5 min, flow rate=30 mL/min) and lyophilized to provide 537 (0.160 g, 38.8% yield). (LCMS) m/z (M+H)⁺=546.3. ¹H NMR (400 MHz, (CD₃)₂SO) δ 8.22 (s, 1H), 8.04 (t, 1H), 7.42-7.35 (m, 4H), 7.21-7.14 (m, 2H), 7.03-6.96 (m, 2H), 4.93 (d, 1H), 4.64-4.58 (m, 1H), 4.43-4.31 (m, 3H), 4.02 (d, 1H), 3.41-3.38 (m, 1H), 3.15-3.04 (m, 1H), 2.97 (s, 6H), 2.91 (s, 6H), 1.42 (d, 3H), 1.33-1.21 (m, 6H). The chiral purity was determined using SFC analytical method on Chiralcel OD-3 50×4.6 mm×3 μm column; Mobile phase A: CO₂/Mobile phase B: EtOH containing 0.2% NH₃; Isocratic: 40% of Mobile phase B; backpressure: 1500 psi; flow rate: 4 mL/min; column temperature: 35 °C to show a mixture of peak 1 ((SFC-MS) m/z (M+H)⁺=546.25 at retention time: 0.609 minutes, 50.54% ee) and peak 2 ((SFC-MS) m/z (M+H)⁺=546.22 at retention time: 1.040 minutes, 49.46% ee).

### Example 538 and 539

### rel-(R or S)-7-(4-(Dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (538 and 539)

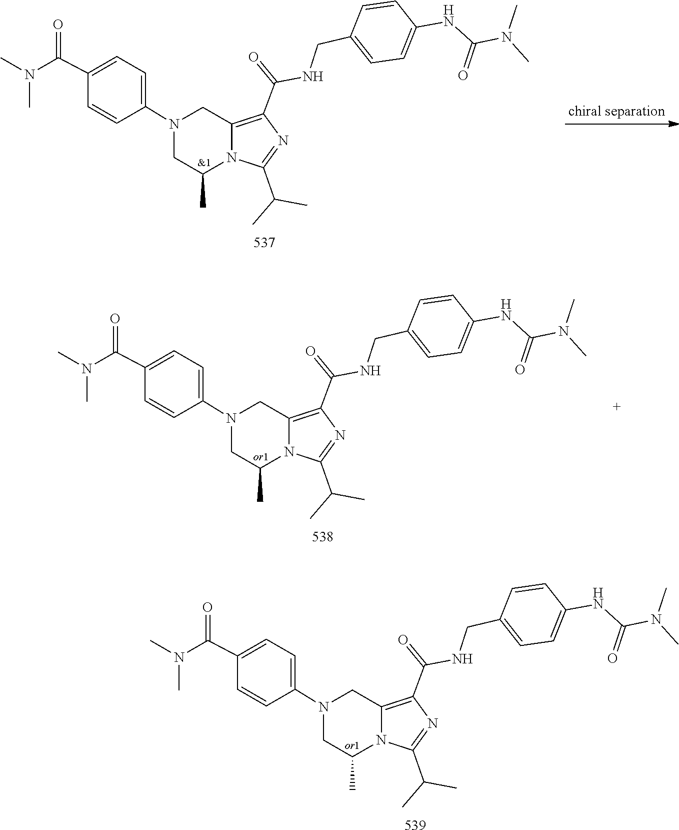

### Step 1. Separation of rel-(R or S)-7-(4-(dimethylcarbamoyl)phenyl)-N-(4-(3,3-dimethylureido)benzyl)-3-isopropyl-5-methyl-5,6,7,8-tetrahydroimidazo[1,5-a]pyrazine-1- PGP-1⁷⁶,C³ carboxamide and rel-(R or S)-7-(4-(dimethylcarbamoyl)phenyl)-N-(4-(3,3-dimethylureido)benzyl)-3-isopropyl-5-methyl-5,6,7,8-tetrahydroimidazo[1,5-a]pyrazine-1-carboxamide (538 and 539)

Example 537 (0.130 g, 0.238 mmol) was purified with chiral SFC [Column: Daicel chiralcel OD C₁₈ 250 mm×30 mm×10 μm, Mobile phase: CO₂/EtOH (0.1% NH₄OH), 60% isocratic over 45 min, flow rate=80 mL/min] and lyophilized to provide the first eluting isomer as 539 (28.6 mg, 22.0% yield) and the second eluting isomer as 538 (27.3 mg, 21.0% yield). The chiral purity was determined using SFC analytical method on Chiralcel OD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35 °C, backpressure: 1500 psi to provide Example 539: peak 1 ((SFC-MS) m/z (M+H)⁺=546.24 at retention time: 0.607 min, 100% ee). ¹H NMR (400 MHz, (CD₃)₂SO) δ 8.19 (s, 1H), 8.02 (t, 1H), 7.38-7.28 (m, 4H), 7.18-7.07 (m, 2H), 7.00-6.91 (m, 2H), 4.88 (d, 1H), 4.60-4.52 (m, 1H), 4.38-4.26 (m, 3H), 3.97 (d, 1H), 3.42-3.38 (m, 1H), 3.11-3.00 (m, 1H), 2.92 (s, 6H), 2.86 (s, 6H), 1.36 (d, 3H), 1.27-1.17 (m, 6H) and Example 538: peak 2 ((SFC-MS) m/z (M+H)⁺=546.22 at retention time: 1.034 min, 100% ee). ¹H NMR (400 MHz, (CD₃)₂SO) δ 8.19 (s, 1H), 8.02 (t, 1H), 7.38-7.31 (m, 4H), 7.17-7.10 (m, 2H), 6.99-6.92 (m, 2H), 4.89 (d, 1H), 4.62-4.52 (m, 1H), 4.40-4.24 (m, 3H), 3.98 (d, 1H), 3.39-3.35 (m, 1H), 3.13-3.00 (m, 1H), 2.93 (s, 6H), 2.87 (s, 6H), 1.37 (d, 3H), 1.29-1.17 (m, 6H).

### Example 546

### rac-(R)-5-(5-(Dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (546)

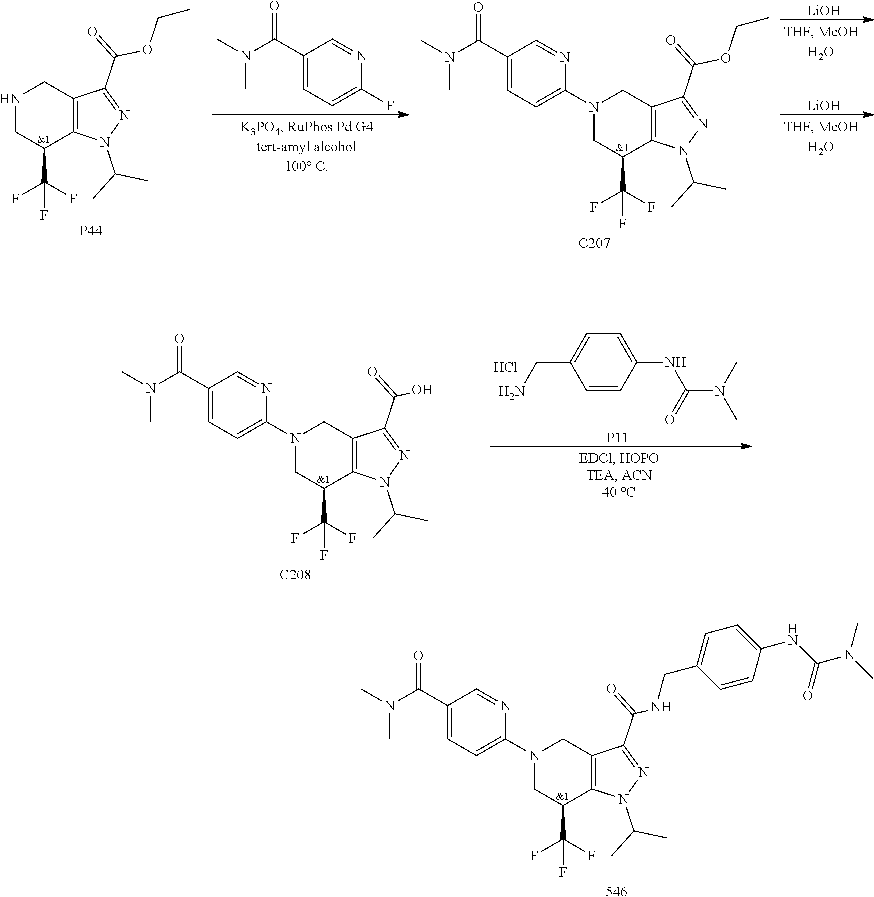

### Step 1. Preparation of rac-ethyl (R)-5-(5-(dimethylcarbamoyl)pyridin-2-yl)-1-isopropyl-7-(trifluoromethyl)-4,5,6,7-tetrahydro-1H-pyrazolo[4,3-c]pyridine-3-carboxylate (C207)

A reaction mixture of P44 (197 mg, 0.576 mmol), 6-chloro-N,N-dimethylnicotinamide (367 mg, 1.99 mmol) and K₃PO₄ (367 mg, 1.73 mmol) in tert-amyl alcohol (3.8 mL) was degassed with nitrogen gas for 5 min. To the suspension was added RuPhos Pd G4 (49.0 mg, 57.6 μmol). The reaction mixture was degassed with nitrogen gas for 5 min then stirred at 100 °C for 16 h. The suspension was diluted with EtOAc (10 mL) then filtered through Celite (1.5 g) prepacked on a plastic filter funnel. The filter cake was eluted with EtOAc. The filtrate was concentrated in vacuo. The residue was dissolved in EtOAc and heptane then concentrated in vacuo (2×). The solid was dissolved in DCM then purified by column chromatography (silica gel, 0-100% EtOAc: heptane) to provide C207 (145 mg, 55.4% yield) as a light-yellow solid. (LC/MS) m/z (M+H)⁺=454.6.

### Step 2. Preparation of rac-(R)-5-(5-(dimethylcarbamoyl)pyridin-2-yl)-1-isopropyl-7-(trifluoromethyl)-4,5,6,7-tetrahydro-1H-pyrazolo[4,3-c]pyridine-3-carboxylic acid

To a solution of C207 (0.140 g, 0.308 mmol) in THF (1.47 mL), MeOH (0.15 mL) and H₂O (1.47 mL) was added LiOH (39.6 mg, 1.65 mmol) at room temperature for 2 h. The reaction mixture was quenched with 1N HCl (1.4 mL) to pH=5-6 then stirred at room temperature for 1.5 h. The reaction mixture was acidified to pH=2-3 with 1 N HCl (1.7 mL) then extracted with DCM (20 mL×1, 2×8 mL). The organic layers were combined then dried with Na₂SO₄, filtered, concentrated in vacuo and dried further under high vacuum to provide C208 (0.144 g, crude) as a light-yellow solid. (LC/MS) m/z (M+H)⁺=426.8. ¹H NMR (400 MHz, CDCl₃) δ 8.40 (d, 1H), 7.73 (dd, 1H), 6.78 (d, 1H), 4.66-4.55 (m, 1H), 4.40 (d, 1H), 3.79-3.66 (m, 3H), 3.31-3.23 (m, 1H), 3.11 (s, 6H), 1.60 (d, 3H), 1.52 (d, 3H).

### Step 3. Preparation of rac-(R)-5-(5-(dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (546)

A reaction mixture of C₂₀₈ (0.060 g, 0.14 mmol), HOPO (19 mg, 0.17 mmol) and EDCI (41 mg, 0.21 mmol) in ACN (0.94 mL) was stirred at 40 °C for 30 min. After the stir, P11 (36 mg, 0.16 mmol) and TEA (43 mg, 0.42 mmol) were added. The reaction mixture was stirred at room temperature for 20 h. The residue was diluted with saturated NaHCO₃ (8 mL) and extracted with EtOAc (3×8 mL). The organic layers were combined then washed with brine then dried with Na₂SO₄, filtered and the filtrate was concentrated in vacuo. The residue was purified by column chromatography (silica gel, 0-55% MeOH: EtOAc) to give 546 (73 mg, 86% yield) as a white solid.

A portion of 546 (16 mg) was re-purified with achiral reverse phase HPLC (XBridge C₁₈ 100 mm×19 mm×5 μm column, Mobile Phase A: H₂O (0.05% TFA)/Mobile Phase B: ACN (0.05% TFA), 5 to 95% of Mobile Phase B over 9 min, 100% of Mobile Phase B hold 1 min, flow rate=25 mL/min) and lyophilized to provide 546 (14 mg, 85% yield). (LC/MS) m/z (M+H)⁺=601.9. ¹H NMR (600 MHz, (CD₃)₂SO) δ 8.57-8.52 (m, 1H), 8.29-8.27 (m, 1H), 8.26-8.22 (m, 1H), 7.72-7.67 (m, 1H), 7.43-7.38 (m, 2H), 7.23-7.15 (m, 2H), 6.87-6.82 (m, 1H), 4.72-4.65 (m, 1H), 4.56-4.50 (m, 1H), 4.41-4.33 (m, 2H), 4.31-4.26 (m, 1H), 4.13-4.07 (m, 1H), 3.22-3.15 (m, 2H), 2.99 (s, 6H), 2.93-2.90 (m, 6H), 1.48-1.40 (m, 6H).

### Example 547 and 548

### rel-(R or S)-5-(5-(Dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (547 and 548)

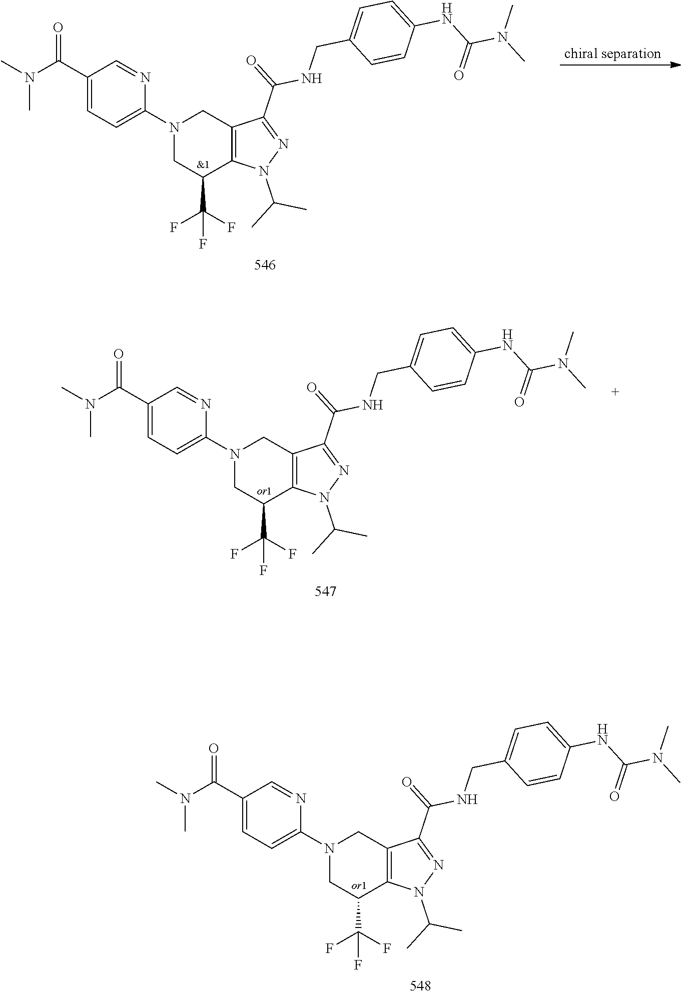

### Step 1. Separation of rel-(R or S)-5-(5-(dimethylcarbamoyl)pyridin-2-yl)-N-(4-(3,3-dimethylureido)benzyl)-1-isopropyl-7-(trifluoromethyl)-4,5,6,7-tetrahydro-1H-pyrazolo[4,3-c]pyridine-3-carboxamide (547 and 548)

546 (56 mg) was purified with chiral SFC [Column: Chiral Technologies OD-H C₁₈ 250 mm×21 mm×5 μm, Mobile phase: (3:2) CO₂/MeOH (0.2% NH₄OH), isocratic over 10 min, flow rate=75 mL/min; backpressure: 120 bar] and lyophilized to provide the first eluting isomer as 548 (22 mg, 38% yield) and the second eluting isomer as 547 (21 mg, 38% yield). The chiral purity was determined using SFC analytical method on Chiral Technologies OD-H 100 mm×4.6 mm×5 μm column; Mobile phase A: CO₂; Mobile phase B: MeOH (0.2% NH₄OH); isocratic gradient: 50% of Mobile Phase B; flow rate: 5.0 mL/min; backpressure: 120 bar to provide Example 548: peak 1 ((SFC-MS) m/z (M+H)⁺=601.3 at retention time: 1.99 min).and Example 547: peak 2 ((SFC-MS) m/z (M+H)⁺=601.3 at retention time: 3.94 min). 547: (LC/MS) m/z (M+H)⁺=601.3. ¹H NMR (600 MHz, (CD₃)₂SO) δ 8.55 (t, 1H), 8.29-8.27 (m, 1H), 8.24 (s, 1H), 7.69 (dd, 1H), 7.42-7.38 (m, 2H), 7.21-7.17 (m, 2H), 6.84 (d, 1H), 4.72-4.65 (m, 1H), 4.57-4.49 (m, 1H), 4.42-4.32 (m, 2H), 4.31-4.25 (m, 1H), 4.16-4.06 (m, 1H), 3.30-3.27 (m, 1H), 3.18-3.15 (m, 1H), 2.99 (s, 6H), 2.91 (s, 6H), 1.44 (dd, 6H).

### Example 561

### 5-(5-(Dimethylcarbamoyl)pyridin-2-yl)-N-(4-(3,3-dimethylureido) 2,3,5,6-d₄-benzyl)-1-isopropyl-4,5,6,7-tetrahydro-1H-pyrazolo[4,3-c]pyridine-3-carboxamide (561)

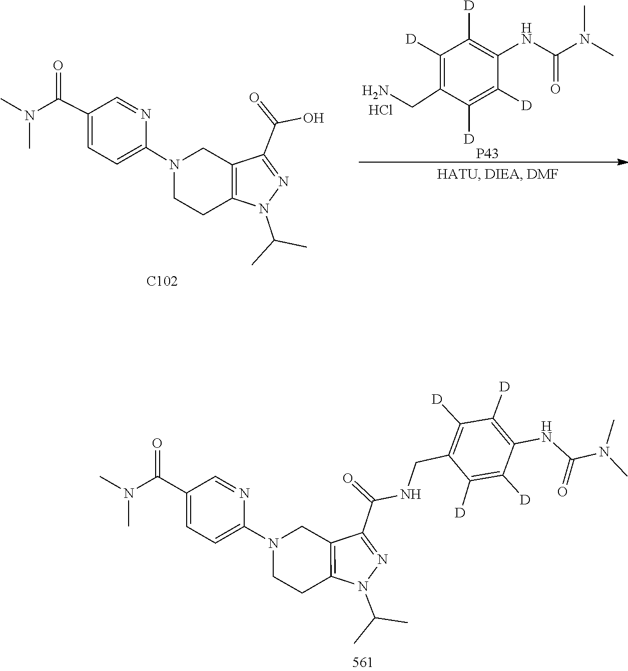

### Step 1. Preparation of 5-(5-(dimethylcarbamoyl)pyridin-2-yl)-N-(4-(3,3-dimethylureido) 2,3,5,6-d₄-benzyl)-1-isopropyl-4,5,6,7-tetrahydro-1H-pyrazolo[4,3-c]pyridine-3-carboxamide (561)

To a solution of C102 (0.940 g, 2.63 mmol) and P43 (1.05 g, 3.16 mmol) in DMF (10 mL) were added DIEA (1.36 g, 10.5 mmol) and HATU (1.10 g, 2.89 mmol). The reaction mixture was stirred at room temperature for 2 h then poured into H₂O (30 mL) and extracted with EtOAc: THF (2:1, 4×30 mL). The combined organic layer was washed with brine (3×50 mL) then dried with MgSO₄ and concentrated in vacuo. The residue was purified by column chromatography (silica gel, 5% MeOH: DCM). The resultant residue was slurried with MeOH (20 mL) at room temperature for 30 min then filtered. The filter cake was dried in vacuo then collected to provide 561 (0.509 g, 34.5% yield) as a white solid. (LC/MS) m/z (M+H)⁺=537.3. ¹H NMR (400 MHz, (CD₃)₂SO) δ 8.32 (t, 1H), 8.26-8.19 (m, 2H), 7.63 (dd, 1H), 6.88 (d, 1H), 4.75 (s, 2H), 4.55-4.43 (m, 1H), 4.35 (d, 2H), 3.95 (t, 2H), 2.97 (s, 6H), 2.91 (s, 6H), 2.82 (t, 2H), 1.39 (d, 6H).

### Example 567

### (S)-5-(4-(Dimethylcarbamoyl)phenyl)-1-isopropyl-7-methyl-N-(4-(4-methyl-1,4-diazepane-1-carboxamido)benzyl)-4,5,6,7-tetrahydro-1H-pyrazolo[4,3-c]pyridine-3-carboxamide (567)

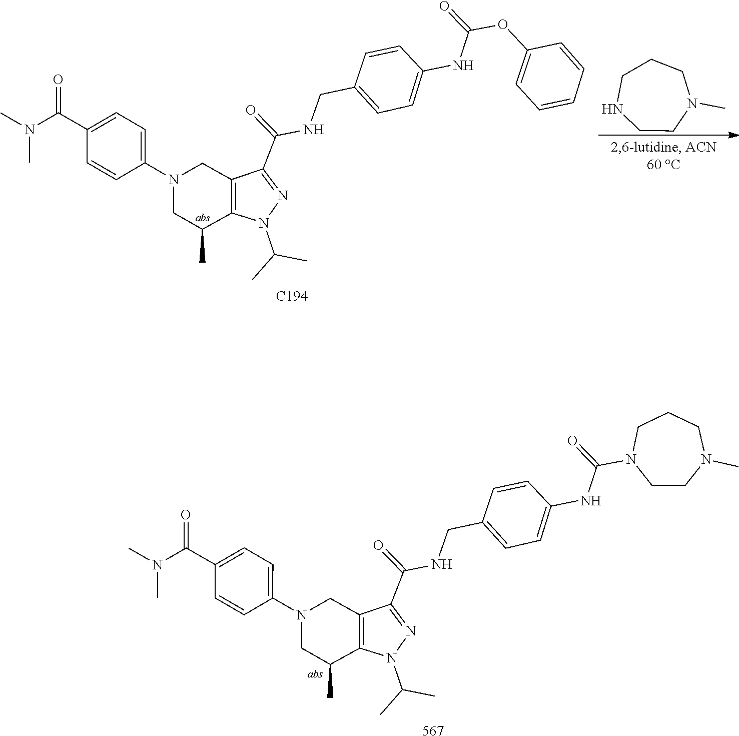

### Step 1. Preparation of (S)-5-(4-(dimethylcarbamoyl)phenyl)-1-isopropyl-7-methyl-N-(4-(4-methyl-1,4-diazepane-1-carboxamido)benzyl)-4,5,6,7-tetrahydro-1H-pyrazolo[4,3-c]pyridine-3-carboxamide (567)

To a reaction mixture of C194 (0.050 g, 0.079 mmol) and 1-methylhomopiperazine (CAS: 4318-37-0; 18 mg, 0.16 mmol) in ACN (0.8 mL) was added 2,6-lutidine (25 mg, 0.24 mmol). The suspension was stirred at 60 °C for 2 h then concentrated in vacuo. The residue was dissolved in DMSO then filtered and purified by reverse phase HPLC (Sunfire C₁₈ 100 mm×19 mm×5 μm column, Mobile Phase A: H₂O (0.05% TFA)/Mobile Phase B: ACN (0.05% TFA), 5 to 95% of Mobile Phase B over 10 min, flow rate=25 mL/min) and lyophilized to provide 567 (37 mg, 76% yield). (LC/MS) m/z (M+H)⁺=615.6.

### Example E427A

### rac-(R)-5-(5-(Dimethylcarbamoyl)pyridin-2-yl)-N-(4-(3,3-dimethylureido)benzyl)-1-ethyl-7,7-dimethyl-4,5,6,7-tetrahydro-1H-indazole-3-carboxamide (E427A)

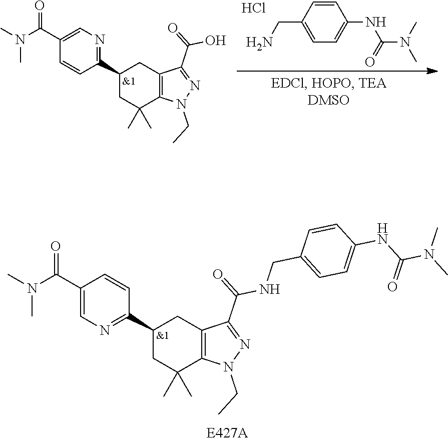

### Step 1. Preparation of rac-(R)-5-(5-(dimethylcarbamoyl)pyridin-2-yl)-N-(4-(3,3-dimethylureido)benzyl)-1-ethyl-7,7-dimethyl-4,5,6,7-tetrahydro-1H-indazole-3-carboxamide (E427A)

To a solution of rac-(R)-5-(5-(dimethylcarbamoyl)pyridin-2-yl)-1-ethyl-7,7-dimethyl-4,5,6,7-tetrahydro-1H-indazole-3-carboxylic acid (52 mg, 0.14 mmol) in DMSO (0.5 mL) was added HOPO (18 mg, 0.16 mmol) and EDCI (35 mg, 0.18 mmol). The suspension was stirred at room temperature for 4 h then 3-(4-(aminomethyl)phenyl)-1,1-dimethylurea hydrochloride (35 mg, 0.35 mmol) and TEA (36 mg, 49 μL, 0.35 mmol) were added. The mixture was stirred at room temperature then diluted with H₂O (4.7 mL). The mixture was extracted with DCM (3×1 mL). The combined organic layers were concentrated in vacuo to form a brown oil. The oil was purified by reverse phase HPLC (Sunfire C₁₈ 100 mm×19 mm×5 μm column; Mobile Phase A: Column: 0.05% TFA in H₂O; Mobile Phase B: 0.05% TFA in ACN; gradient: 5-45% of Mobile Phase B over 8.5 min then 45-95% of Mobile Phase B over 0.5 min then held at 95% of Mobile Phase B for 1.0 min; flow rate: 25 mL/min) to provide rac-(R)-5-(5-(dimethylcarbamoyl)pyridin-2-yl)-N-(4-(3,3-dimethylureido)benzyl)-1-ethyl-7,7-dimethyl-4,5,6,7-tetrahydro-1H-indazole-3-carboxamide (41 mg, 53% yield) as a gum. (LC/MS) m/z (M+H)⁺=546.4. ¹H NMR (600 MHz, (CD₃)₂SO) δ 8.64-8.61 (m, 1H), 8.29 (t, 1H), 8.22 (s, 1H), 7.92-7.87 (m, 1H), 7.53 (d, 1H), 7.40-7.35 (m, 2H), 7.17-7.12 (m, 2H), 4.30 (d, 2H), 4.29-4.15 (m, 2H), 3.26-3.17 (m, 2H), 3.02 (s, 3H), 2.96 (s, 3H), 2.91 (s, 6H), 2.71-2.63 (m, 1H), 2.00 (t, 1H), 1.88-1.82 (m, 1H), 1.45-1.39 (m, 6H), 1.38 (s, 3H)

### Example E428A and E429A

### rel-(S)-5-(5-(Dimethylcarbamoyl)pyridin-2-yl)-N-(4-(3,3-dimethylureido)benzyl)-1-ethyl-7,7-dimethyl-4,5,6,7-tetrahydro-1H-indazole-3-carboxamide and rel-(R)-5-(5-(Dimethylcarbamoyl)pyridin-2-yl)-N-(4-(3,3-dimethylureido)benzyl)-1-ethyl-7,7-dimethyl-4,5,6,7-tetrahydro-1H-indazole-3-carboxamide (E428A and E429A)

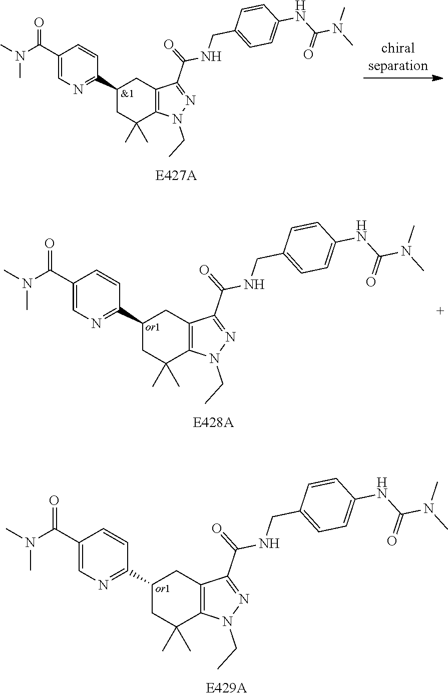

### Step 1. Preparation of rel-(S)-5-(5-(dimethylcarbamoyl)pyridin-2-yl)-N-(4-(3,3-dimethylureido)benzyl)-1-ethyl-7,7-dimethyl-4,5,6,7-tetrahydro-1H-indazole-3-carboxamide and rel-(R)-5-(5-(dimethylcarbamoyl)pyridin-2-yl)-N-(4-(3,3-dimethylureido)benzyl)-1-ethyl-7,7-dimethyl-4,5,6,7-tetrahydro-1H-indazole-3-carboxamide (E428A and E429A)

Example E427 (0.698 g, 1.28 mmol) was separated into its component isomers using SFC purification (Chiral Technologies OD-H 250 mm×30 mm×5 μm column; Mobile phase A: CO₂; Mobile phase B: (1:1, ACN: MeOH); isocratic: 40% of Mobile Phase B; flow rate: 80 mL/min; column temperature: 40 °C.; backpressure: 120 bar) and lyophilized to provide Example E428A: peak 1 (163 mg, 23.3% yield) as a white gum and Example E429A: peak 2 (0.180 g, 25.8% yield) as a white gum.

The chiral purity were determined using SFC analytical method on Chiral Technologies OD-H 250 mm×4.6 mm×5 μm column; Mobile phase A: CO₂; Mobile phase B: (1:1, ACN: MeOH); gradient: held at 5% of Mobile Phase B for 0.50 min then 5-100% of Mobile Phase B in 5.00 min then held at 100% of Mobile Phase B for 2.00 min, then 100-5% of Mobile Phase B over 0.50 min; flow rate: 3.0 mL/min; backpressure: 120 bar; column temperature: 40 °C to provide Example E428A: peak 1 ((SFC-MS) retention time: 4.622 min, 100% ee) and Example E429A: peak 2 ((SFC-MS) retention time: 4.873 min, 100% ee). E428A: ¹H NMR (400 MHz, (CD₃)₂SO) δ 8.60-8.54 (m, 1H), 8.26 (t, 1H), 8.21 (s, 1H), 7.80 (dd, 1H), 7.48-7.40 (m, 1H), 7.40-7.33 (m, 2H), 7.18-7.10 (m, 2H), 4.32-4.12 (m, 4H), 3.22-3.14 (m, 2H), 3.00 (s, 3H), 2.95 (s, 3H), 2.90 (s, 6H), 2.65 (dd, 1H), 1.97 (t, 1H), 1.84 (d, 1H), 1.45-1.35 (m, 9H).

E429A: ¹H NMR (400 MHz, (CD₃)₂SO) δ 8.60-8.55 (m, 1H), 8.26 (t, 1H), 8.21 (s, 1H), 7.80 (dd, 1H), 7.48-7.40 (m, 1H), 7.42-7.33 (m, 2H), 7.21-7.08 (m, 2H), 4.31-4.12 (m, 4H), 3.23-3.14 (m, 2H), 3.00 (s, 3H), 2.93 (s, 3H), 2.90 (s, 6H), 2.65 (dd, 1H), 1.97 (t, 1H), 1.84 (d, 1H), 1.45-1.35 (m, 9H).

### Example E430A

### rac-(5R,7S)-5-(5-(Dimethylcarbamoyl)pyridin-2-yl)-N-(4-(3,3-dimethylureido)benzyl)-1-ethyl-7-methyl-4,5,6,7-tetrahydro-1H-indazole-3-carboxamide (E430A)

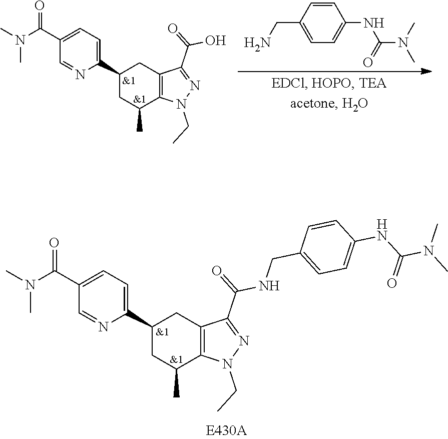

### Step 1. Preparation of rac-(5R,7S)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide (E430A)

A reaction mixture of rac-(5R,7R)-5-(5-(dimethylcarbamoyl)pyridin-2-yl)-1-ethyl-7-methyl-4,5,6,7-tetrahydro-1H-indazole-3-carboxylic acid (545 mg, 1.53 mmol), EDCI (440 mg, 2.29 mmol), HOPO (204 mg, 1.83 mmol), acetone (3 mL), and H₂O (3 mL) was stirred at room temperature for 30 min before 3-(4-(aminomethyl)phenyl)-1,1-dimethylurea (387 mg, 1.68 mmol) and TEA (464 mg, 639 μL, 4.59 mmol) were added. The reaction mixture was stirred at room temperature for 1.5 h then diluted with H₂O (20 mL) and extracted with 10% MeOH in DCM (3×30 mL). The combined organic layers were dried over Na₂SO₄, filtered, and concentrated in vacuo. A portion of the crude product (26 mg, 0.049 mmol) was purified by reverse phase HPLC (Sunfire C₁₈ 100 mm×19 mm×5 μm, Mobile Phase A: H₂O (0.05% TFA)/Mobile Phase B: ACN (0.05% TFA), 5-40% of Mobile Phase B over 10 min, flow rate=25 mL/min) to provide rac-(5R,7S)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide (17.6 mg).

The remaining crude was purified by column chromatography (silica gel, 0-20% MeOH: DCM) to afford rac-(5R,7S)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide (0.615 g) as a white solid. The desired products were combined to provide rac-(5R,7S)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide (0.633 g, 77.9% yield) as a white solid. (LC/MS) m/z (M+H)⁺=532.6. ¹H NMR (600 MHz, (CD₃)₂SO) δ 8.61-8.57 (m, 1H), 8.31-8.25 (m, 1H), 8.21 (s, 1H), 7.86-7.80 (m, 1H), 7.44 (d, 1H), 7.39-7.34 (m, 2H), 7.16-7.11 (m, 2H), 4.29 (d, 2H), 4.22-4.09 (m, 2H), 3.23-3.07 (m, 2H), 3.07-2.93 (m, 7H), 2.90 (s, 6H), 2.73-2.65 (m, 1H), 2.26-2.20 (m, 1H), 1.78-1.69 (m, 1H), 1.42-1.35 (m, 3H), 1.31-1.27 (m, 3H).

### Example E431A, E432A, E433A and E434A

### rel-(5R,7S)-5-(5-(Dimethylcarbamoyl)pyridin-2-yl)-N-(4-(3,3-dimethylureido)benzyl)-1-ethyl-7-methyl-4,5,6,7-tetrahydro-1H-indazole-3-carboxamide, rel-(5R,7R)-5-(5-(Dimethylcarbamoyl)pyridin-2-yl)-N-(4-(3,3-dimethylureido)benzyl)-1-ethyl-7-methyl-4,5,6,7-tetrahydro-1H-indazole-3-carboxamide, rel-(5S,7S)-5-(5-(Dimethylcarbamoyl)pyridin-2-yl)-N-(4-(3,3-dimethylureido)benzyl)-1-ethyl-7-methyl-4,5,6,7-tetrahydro-1H-indazole-3-carboxamide and rel-(5S,7R)-5-(5-(Dimethylcarbamoyl)pyridin-2-yl)-N-(4-(3,3-dimethylureido)benzyl)-1-ethyl-7-methyl-4,5,6,7-tetrahydro-1H-indazole-3-carboxamide (E431A, E432A, E433A and E434A)

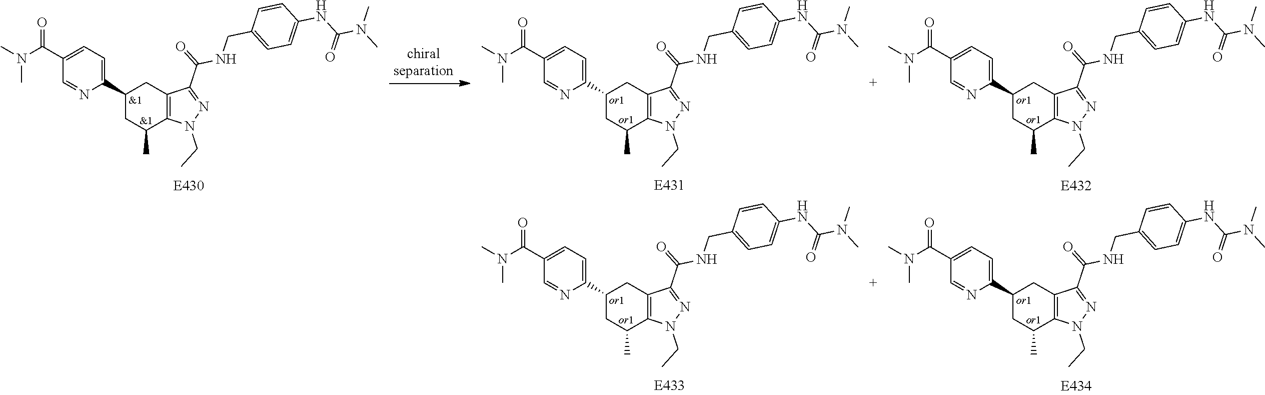

### Step 1. Preparation of rel-(5R,7S)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide, rel-(5R,7R)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide, rel-(5S,7S)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide and rel-(5S,7R)-5-(5-(dimethylcarbamoyl)pyridin-2-yl)-N-(4-(3,3-dimethylureido)benzyl)-1-ethyl-7-methyl-4,5,6,7-tetrahydro-1H-indazole-3-carboxamide (E431A, E432A, E433A and E434A)

Example E430A (0.615 g, 1.16 mmol) was purified by chiral SFC [Column: Chiral Technologies AD-H C₁₈ 250 mm×30 mm×5 μm, Mobile phase A: CO₂/Mobile Phase B: (1:1)(ACN: MeOH), isocratic at 40% of Mobile Phase B, flow rate=100 mL/min; backpressure: 100 bar, temperature=40 °C.] to provide peak 1: Example E431A (42.0 mg, 6.83% yield), peak 2: Example E432A (242 mg, 39.3% yield) and peak 3 was a mixture of isomers.

The third eluting peak was purified by chiral SFC [Column: Chiral Technologies OX-H C₁₈ 250 mm×30 mm×5 μm, Mobile phase A: CO₂/Mobile Phase B: (1:1)(ACN: MeOH), isocratic at 50% of Mobile Phase B, flow rate=100 mL/min; backpressure: 100 bar, temperature=40 °C.] to give first eluting peak: Example E433A (42.9 mg, 6.98% yield) and the second eluting peak: Example E434A (263 mg, 42.8% yield).

The chiral purity for Example E431A and E432A were determined using SFC analytical method on [Chiral Technologies AD-H 250 mm×4.6 mm×5 μm column; Mobile phase A: CO₂; Mobile phase B: (1:1)(ACN: MeOH); gradient: held at 5% of Mobile Phase B over 1.00 min then 5-100% of Mobile Phase B over 4.50 min then held at 100% of Mobile Phase B over 2.00 min then 100-5% of Mobile Phase B over 0.50 min; flow rate: 3.0 mL/min; backpressure: 100 bar; temperature: 40 °C.] to provide Example E431A: peak 1 ((HPLC) retention time: 5.528 min, 100% ee).and Example E432A: peak 2 ((HPLC) retention time: 5.467 min, 98% ee).

The chiral purity for Example E433A and E434A were determined using SFC analytical method on [Chiral Technologies OX-H 250 mm×4.6 mm×5 μm column; Mobile phase A: CO₂; Mobile phase B: (1:1)(ACN: MeOH); gradient: held at 5% of Mobile Phase B over 1.00 min then 5-100% of Mobile Phase B over 4.50 min then held at 100% of Mobile Phase B over 2.00 min then 100-5% of Mobile Phase B over 0.50 min; flow rate: 3.0 mL/min; backpressure: 100 bar; temperature: 40 °C.] to provide Example E433A: peak 3 ((HPLC) retention time: 6.137 min, 100% ee).and Example E434A: peak 4 ((HPLC) retention time: 6.285 min, 100% ee). Example E431A: (LC/MS) m/z (M+H)⁺=532.6. ¹H NMR (600 MHz, (CD₃)₂SO) δ 8.59-8.55 (m, 1H), 8.26 (t, 1H), 8.21 (s, 1H), 7.83-7.77 (m, 1H), 7.48-7.39 (m, 1H), 7.39-7.34 (m, 2H), 7.16-7.11 (m, 2H), 4.30 (d, 2H), 4.12-4.00 (m, 2H), 3.28-3.09 (m, 3H), 3.00 (s, 3H), 2.95 (s, 3H), 2.90 (s, 6H), 2.68 (dd, 1H), 2.16-2.08 (m, 1H), 2.01-1.97 (m, 1H), 1.42-1.36 (m, 3H), 1.29 (d, 3H). Example E432A: (LC/MS) m/z (M+H)⁺=532.6. ¹H NMR (600 MHz, (CD₃)₂SO) δ 8.59-8.55 (m, 1H), 8.27 (t, 1H), 8.21 (s, 1H), 7.80 (dd, 1H), 7.44-7.39 (m, 1H), 7.39-7.34 (m, 2H), 7.16-7.11 (m, 2H), 4.30 (d, 2H), 4.22-4.09 (m, 2H), 3.19-3.07 (m, 2H), 3.06-2.91 (m, 7H), 2.90 (s, 6H), 2.73-2.65 (m, 1H), 2.26-2.20 (m, 1H), 1.77-1.68 (m, 1H), 1.39 (t, 3H), 1.29 (d, 3H). Example E433A: (LC/MS) m/z (M+H)⁺=532.5. ¹H NMR (600 MHz, (CD₃)₂SO) δ 8.59-8.55 (m, 1H), 8.27 (t, 1H), 8.21 (s, 1H), 7.80 (dd, 1H), 7.41 (d, 1H), 7.39-7.34 (m, 2H), 7.16-7.12 (m, 2H), 4.30 (d, 2H), 4.22-4.09 (m, 2H), 3.19-3.08 (m, 1H), 3.05-2.92 (m, 8H), 2.90 (s, 6H), 2.73-2.65 (m, 1H), 2.26-2.19 (m, 1H), 1.77-1.68 (m, 1H), 1.39 (t, 3H), 1.29 (d, 3H). Example E434A: (LC/MS) m/z (M+H)⁺=532.5. ¹H NMR (600 MHz, (CD₃)₂SO) δ 8.59-8.55 (m, 1H), 8.26 (t, 1H), 8.21 (s, 1H), 7.81 (dd, 1H), 7.46 (d, 1H), 7.39-7.34 (m, 2H), 7.16-7.11 (m, 2H), 4.32-4.28 (m, 2H), 4.12-4.01 (m, 2H), 3.29-3.09 (m, 3H), 3.03-2.94 (m, 6H), 2.90 (s, 6H), 2.68 (dd, 1H), 2.16-2.08 (m, 1H), 1.88 (d, 1H), 1.39 (t, 3H), 1.29 (d, 3H).

### Examples 10-758

The compounds in Table 1 were prepared using similar chemistry as described above in the selected Examples 1-567 that are commercially available or that can be synthesized by literature methods to form the resultant compounds of the disclosure.

TABLE 1

| | Method | | | LC/MS |
|---|---|---|---|---|
| | of | | | m/z |
| Ex | Synthesis | Structure/NMR | ChemDraw Name | (M + H)⁺ |
| 10 | Ex. 10 | 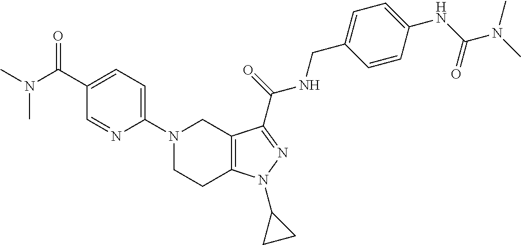 ¹H NMR* (600 MHz, (CD₃)₂SO) δ 8.46 (t, 1H), 8.22-8.21 (m, 2H), 7.68 (dd, 1H), 7.40- 7.34 (m, 2H), 7.15 (d, 2H), 6.96 (d, 1H), 4.75 (s, 2H), 4.32 (d, 2H), 3.96 (t, 2H), 3.56 (tt, 1H), 2.97 (s, 6H), 2.91-2.86 (m, 8H), 1.12-1.05 (m, 2H), 1.01-0.95 (m, 2H). | 1-cyclopropyl-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 531.5 |
| 11 | Ex. 10 | 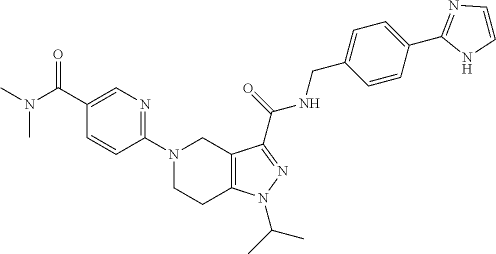 [SMILES: CC(C)n1nc(C(=O)NCc2ccc(-c3ncc[nH]3)cc2)c2c1CCN(c1ccc(C(=O)N(C)C)cn1)C2] ¹H NMR* (600 MHz, (CD₃)₂SO) δ 14.61 (br s, 1H), 8.62 (t, 1H), 8.23 (d, 1H), 7.96- 7.92 (m, 2H), 7.82 (s, 2H), 7.64 (dd, 1H), 7.57 (d, 2H), 6.89 (d, 1H), 4.74 (s, 2H), 4.56- 4.48 (m, 3H), 3.95 (t, 2H), 2.99-2.94 (m, 6H), 2.84 (t, 2H), 1.41 (d, 6H). | N-(4-(1H-imidazol-2- yl)benzyl)-5-(5- (dimethylcarbamoyl) pyridin-2-yl)-1-isopropyl- 4,5,6,7-tetrahydro-1H- pyrazolo[4,3-c]pyridine- 3-carboxamide | 513.4 |
| 12 | Ex. 12 | 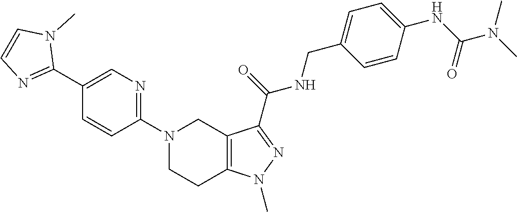 [SMILES: CN(C)C(=O)Nc1ccc(CNC(=O)c2nn(C)c3c2CN(c2ccc(-c4nccn4C)cn2)CC3)cc1] ¹H NMR (600 MHz, (CD₃)₂SO) δ 8.47 (t, 1H), 8.43 (d, 1H), 8.22 (s, 1H), 7.84 (dd, 1H), 7.42-7.34 (m, 2H), 7.22 (s, 1H), 7.16 (d, 2H), 7.01-6.93 (m, 2H), 4.77 (s, 2H), 4.32 (d, 2H), 3.96 (t, 2H), 3.76 (s, 3H), 3.71 (s, 3H), 2.90 (s, 6H), 2.80 (t, 2H). | N-(4-(3,3- dimethylureido)benzyl)- 1-methyl-5-(5-(1-methyl- 1H-imidazol-2-yl)pyridin- 2-yl)-4,5,6,7-tetrahydro- 1H-pyrazolo[4,3- c]pyridine-3- carboxamide | 514.4 |
| 13 | Ex. 5 | 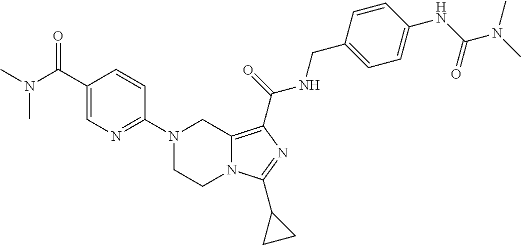 ¹H NMR (400 MHz, CD₃OD) δ 8.31 (d, 1H), 7.73 (dd, 1H), 7.35-7.23 (m, 4H), 6.95 (d, 1H), 5.04 (s, 2H), 4.47 (s, 2H), 4.20 (s, 4H), 3.09 (s, 6H), 3.01 (s, 6H), 1.97-1.88 (m, 1H), 1.03-0.89 (m, 4H). | 3-cyclopropyl-7-(5- (dimethylcarbamoyl) pyridin-2-yl)-N-(4-(3,3- dimethylureido)benzyl)- 5,6,7,8- tetrahydroimidazo[1,5- a]pyrazine-1- carboxamide | 531.5 |
| 14 | Ex. 5 | 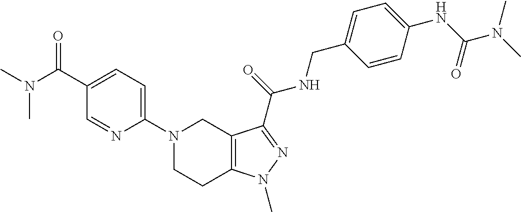 ¹H NMR* (600 MHz, (CD₃)₂SO) δ 8.48 (t, 1H), 8.25-8.18 (m, 2H), 7.66 (dd, 1H), 7.37 (d, 2H), 7.15 (d, 2H), 6.93 (d, 1H), 4.76 (s, 2H), 4.32 (d, 2H), 3.95 (t, 2H), 3.75 (s, 6H), 2.96 (s, 3H), 2.90 (s, 6H), 2.79 (t, 2H). | 5-(5- (dimethylcarbamoyl) pyridin-2-yl)-N-(4-(3,3- dimethylureido)benzyl)- 1-methyl-4,5,6,7- tetrahydro-1H- pyrazolo[4,3-c]pyridine- 3-carboxamide | 505.5 |
| 15 | Ex. 5 | 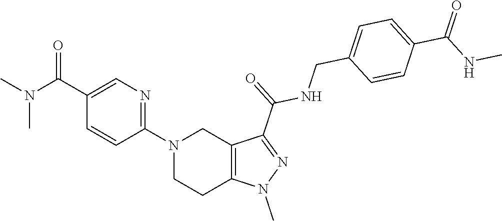 ¹H NMR (400 MHz, CD₃OD) δ 8.27-8.25 (m, 1H), 7.80-7.75 (m, 2H), 7.66 (dd, 1H), 7.44 (d, 2H), 6.89 (d, 1H), 4.77 (s, 2H), 4.59 (s, 2H), 4.07 (t, 2H), 3.80 (s, 3H), 3.08 (s, 6H), 2.91 (s, 3H), 2.83 (t, 2H). | 5-(5- (dimethylcarbamoyl) pyridin-2-yl)-1-methyl-N-(4- (methylcarbamoyl)benzyl)- 4,5,6,7-tetrahydro-1H- pyrazolo[4,3-c]pyridine- 3-carboxamide | 476.4 |
| 16 | Ex. 5 | 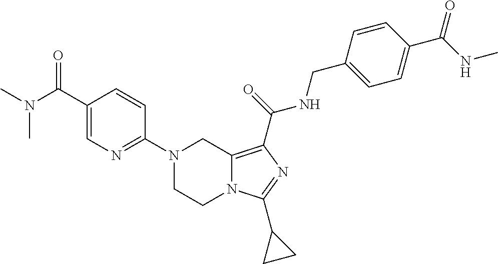 ¹H NMR (400 MHz, CD₃OD) δ 8.31 (d, 1H), 7.79-7.75 (m, 2H), 7.74-7.69 (m, 1H), 7.43 (d, 2H), 6.95 (d, 1H), 5.04 (s, 2H), 4.58 (s, 2H), 4.20 (s, 5H), 3.09 (s, 7H), 2.90 (s, 3H), 1.98-1.89 (m, 1H), 1.03-0.89 (m, 4H). | 3-cyclopropyl-7-(5- (dimethylcarbamoyl) pyridin-2-yl)-N-(4- (methylcarbamoyl)benzyl)- 5,6,7,8- tetrahydroimidazo[1,5- a]pyrazine-1- carboxamide | 502.4 |
| 17 | Ex. 5 | 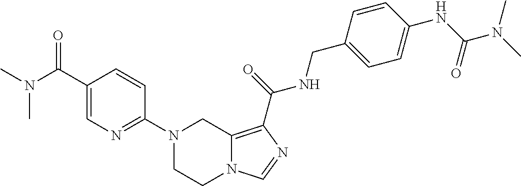 ¹H NMR (400 MHz, CD₃OD) δ 8.32-8.29 (m, 1H), 7.72 (dd, 1H), 7.62 (s, 1H), 7.36- 7.31 (m, 2H), 7.29-7.25 (m, 2H), 6.94 (d, 1H), 5.07 (s, 2H), 4.50 (s, 2H), 4.26-4.11 (m, 4H), 3.09 (s, 6H), 3.00 (s, 6H). | 7-(5- (dimethylcarbamoyl) pyridin-2-yl)-N-(4-(3,3- dimethylureido)benzyl)- 5,6,7,8- tetrahydroimidazo[1,5- a]pyrazine-1- carboxamide | 491.4 |
| 18 | Ex. 18 | 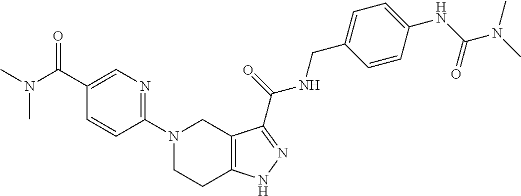 1H NMR (400 MHz, CD₃OD) δ 8.26 (d, J = 2.4 Hz, 1H), 7.67 (dd, J = 8.9, 2.4 Hz, 1H), 7.35-7.25 (m, 4H), 6.89 (d, J = 8.9 Hz, 1H), 4.78 (s, 2H), 4.50 (s, 2H), 4.05 (t, J = 5.6 Hz, 2H), 3.08 (s, 6H), 3.00 (s, 6H), 2.84 (t, J = 5.8 Hz, 2H). | 5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 491.4 |
| 19 | Ex.10 | 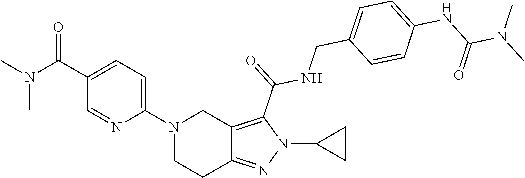 ¹H NMR* (600 MHz, (CD₃)₂SO) δ = 8.69 (t, 1H), 8.29-8.21 (m, 2H), 7.68 (dd, 1H), 7.49-7.42 (m, 2H), 7.27-7.18 (m, 2H), 6.88 (d, 1H), 4.70 (s, 2H), 4.43 (d, 2H), 3.99- 3.87 (m, 3H), 3.00-2.94 (m, 6H), 2.92 (s, 6H), 2.69 (t, 2H), 1.00-0.92 (m, 2H), 0.91- 0.86 (m, 2H). | 2-cyclopropyl-5-(5- (dimethylcarbamoyl) pyridin-2-yl)-N-(4-(3,3- dimethylureido)benzyl)- 4,5,6,7-tetrahydro-2H- pyrazolo[4,3-c]pyridine- 3-carboxamide | 531.6 |
| 20 | Ex. 5 | 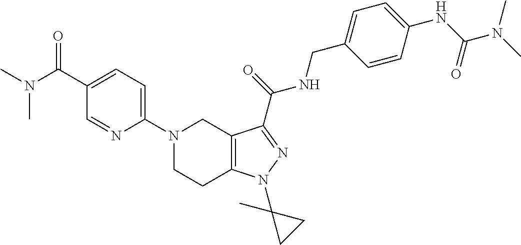 ¹H NMR (400 MHz, CD₃OD) δ 8.26 (d, 1H), 7.67 (dd, 1H), 7.35-7.31 (m, 2H), 7.29- 7.25 (m, 2H), 6.88 (d, 1H), 4.74 (s, 2H), 4.48 (s, 2H), 4.06 (t, 2H), 3.08 (s, 6H), 3.01- 2.99 (m, 6H), 2.94 (t, 2H), 1.51 (s, 3H), 1.24 (t, 2H), 0.96 (t, 2H). | 5-(5- (dimethylcarbamoyl) pyridin-2-yl)-N-(4-(3,3- dimethylureido)benzyl)- 1-(1-methylcyclopropyl)- 4,5,6,7-tetrahydro-1H- pyrazolo[4,3-c]pyridine- 3-carboxamide | 545.4 |
| 21 | Ex. 5 | 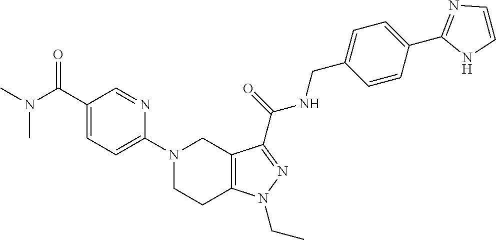 [SMILES: CCn1nc(C(=O)NCc2ccc(-c3ncc[nH]3)cc2)c2c1CCN(c1ccc(C(=O)N(C)C)cn1)C2] ¹H NMR (400 MHz, CD₃OD) δ 8.27 (d, 1H), 7.87-7.81 (m, 2H), 7.67 (dd, 1H), 7.49- 7.44 (m, 2H), 7.14 (s, 2H), 6.90 (d, 1H), 4.78 (s, 2H), 4.59 (s, 2H), 4.20-4.01 (m, 4H), 3.14-3.05 (m, 6H), 2.91-2.82 (m, 2H), 1.42 (t, 3H). | N-(4-(1H-imidazol-2- yl)benzyl)-5-(5- (dimethylcarbamoyl) pyridin-2-yl)-1-ethyl-4,5,6,7- tetrahydro-1H- pyrazolo[4,3-c]pyridine- 3-carboxamide | 499.4 |
| 22 | Ex. 5 | 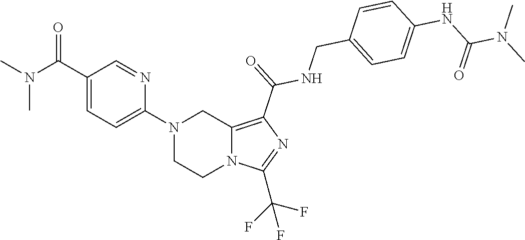 1H NMR (400 MHz, CD₃OD) δ 8.32 (d, 1H), 7.74 (dd, 1H), 7.37-7.31 (m, 2H), 7.31- 7.24 (m, 2H), 6.98 (d, 1H), 5.15 (s, 2H), 4.49 (s, 2H), 4.33 (t, 2H), 4.21 (t, 2H), 3.09 (s, 6H), 3.01 (s, 6H). | 7-(5- (dimethylcarbamoyl) pyridin-2-yl)-N-(4-(3,3- dimethylureido)benzyl)- 3-(trifluoromethyl)- 5,6,7,8- tetrahydroimidazo[1,5- a]pyrazine-1- carboxamide | 559.3 |
| 23 | Ex. 23 | 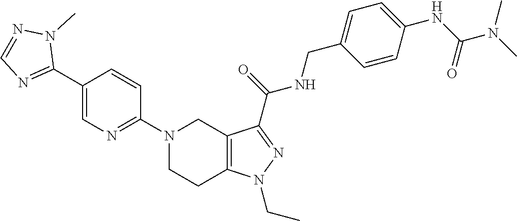 [SMILES: CCn1nc(C(=O)NCc2ccc(NC(=O)N(C)C)cc2)c2c1CCN(c1ccc(-c3ncnn3C)cn1)C2] ¹H NMR (400 MHz, (CD₃)₂SO) δ 8.56 (d, 1H), 8.44 (t, 1H), 8.23 (s, 1H), 7.99-7.92 (m, 2H), 7.45-7.34 (m, 2H), 7.22-7.17 (m, 2H), 7.05 (d, 1H), 4.83 (s, 2H), 4.36 (d, 2H), 4.11 (q, 2H), 4.02 (t, 2H), 3.97 (s, 3H), 2.93 (s, 6H), 2.86 (t, 2H), 1.36 (t, 3H). | N-(4-(3,3- dimethylureido)benzyl)- 1-ethyl-5-(5-(1-methyl- 1H-1,2,4-triazol-5- yl)pyridin-2-yl)-4,5,6,7- tetrahydro-1H- pyrazolo[4,3-c]pyridine- 3-carboxamide | 529.3 |
| 24 | Ex. 5 | 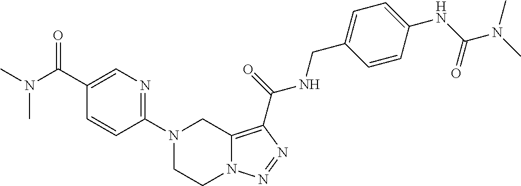 ¹H NMR* (400 MHz, CD₃OD) δ 8.32 (d, 1H), 7.74 (dd, 1H), 7.37-7.32 (m, 2H), 7.28- 7.25 (m, 2H), 7.00 (d, 1H), 5.12 (s, 2H), 4.57-4.50 (m, 4H), 4.25 (t, 2H), 3.08 (s, 6H), 3.00 (s, 6H). | 5-(5- (dimethylcarbamoyl) pyridin-2-yl)-N-(4-(3,3- dimethylureido)benzyl)- 4,5,6,7-tetrahydro- [1,2,3]triazolo[1,5- a]pyrazine-3- carboxamide | 492.3 |
| 25 | Ex. 5 | 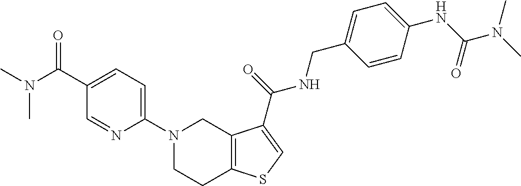 ¹H NMR (400 MHz, CD₃OD) δ 8.25 (d, 1H), 7.79 (s, 1H), 7.66 (dd, 1H), 7.36-7.30 (m, 2H), 7.30-7.23 (m, 2H), 6.86 (d, 1H), 4.83- 4.81 (m, 2H), 4.47 (s, 2H), 4.05 (t, 2H), 3.08 (s, 6H), 3.00 (s, 6H), 2.93 (t, 2H). | 5-(5- (dimethylcarbamoyl) pyridin-2-yl)-N-(4-(3,3- dimethylureido)benzyl)- 4,5,6,7- tetrahydrothieno[3,2- c]pyridine-3- carboxamide | 507.4 |
| 26 | Ex. 26 | 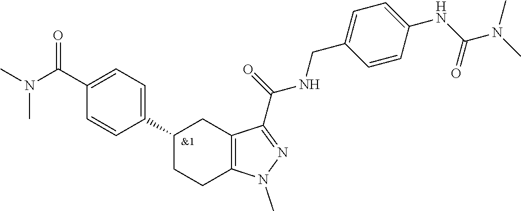 ¹H NMR (400 MHz, (CD₃)₂SO) δ 8.31 (t, 1H), 8.22 (s, 1H), 7.41-7.34 (m, 6H), 7.19- 7.10 (m, 2H), 4.31 (d, 2H), 3.76 (s, 3H), 3.13-3.04 (m, 2H), 2.99-2.87 (m, 12H), 2.86-2.57 (m, 4H), 2.13-1.89 (m, 2H). | rac-(R)-5-(4- (dimethylcarbamoyl) phenyl)-N-(4-(3,3- dimethylureido)benzyl)- 1-methyl-4,5,6,7- tetrahydro-1H-indazole- 3-carboxamide | 503.4 |
| 27 | Ex. 23 | 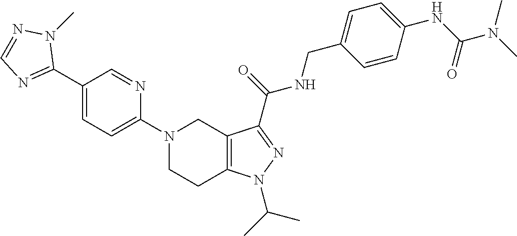 [SMILES: CC(C)n1nc(C(=O)NCc2ccc(NC(=O)N(C)C)cc2)c2c1CCN(c1ccc(-c3ncnn3C)cn1)C2] ¹H NMR (400 MHz, (CD₃)₂SO) δ 8.56 (d, 1H), 8.34 (t, 1H), 8.24 (s, 1H), 7.98-7.92 (m, 2H), 7.45-7.38 (m, 2H), 7.26-7.18 (m, 2H), 7.05 (d, 1H), 4.83 (s, 2H), 4.51- 4.46 (m, 1H), 4.38 (d, 2H), 4.03 (t, 2H), 3.97 (s, 3H), 2.93 (s, 6H), 2.87 (t, 2H), 1.43 (d, 6H). | N-(4-(3,3- dimethylureido)benzyl)- 1-isopropyl-5-(5-(1- methyl-1H-1,2,4-triazol- 5-yl)pyridin-2-yl)-4,5,6,7- tetrahydro-1H- pyrazolo[4,3-c]pyridine- 3-carboxamide | 543.5 |
| 28 | Ex. 5 | 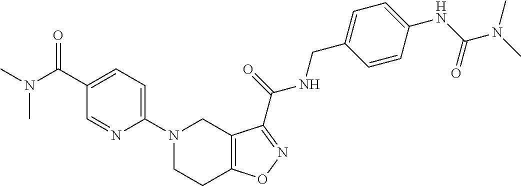 ¹H NMR* (400 MHz, CD₃OD) δ 8.26 (d, 1H), 7.67 (dd, 1H), 7.35-7.29 (m, 2H), 7.29- 7.23 (m, 2H), 6.92 (d, 1H), 4.71 (s, 2H), 4.49 (s, 2H), 4.07 (t, 2H), 3.07 (s, 6H), 2.99 (s, 6H), 2.97-2.89 (t, 2H). | 5-(5- (dimethylcarbamoyl) pyridin-2-yl)-N-(4-(3,3- dimethylureido)benzyl)- 4,5,6,7- tetrahydroisoxazolo[4,5- c]pyridine-3- carboxamide | 492.3 |
| 29 | Ex. 5 | 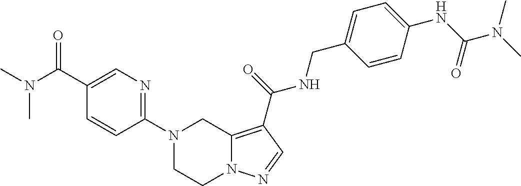 ¹H NMR (400 MHz, CD₃OD) δ 8.34-8.31 (m, 1H), 7.96 (s, 1H), 7.74 (dd, 1H), 7.36-7.32 (m, 2H), 7.29-7.24 (m, 2H), 7.01-6.98 (m, 1H), 5.09 (s, 2H), 4.48 (s, 2H), 4.32-4.24 (m, 4H), 3.10 (s, 6H), 3.02 (d, 6H). | 5-(5- (dimethylcarbamoyl) pyridin-2-yl)-N-(4-(3,3- dimethylureido)benzyl)- 4,5,6,7- tetrahydropyrazolo[1,5- a]pyrazine-3- carboxamide | 491.4 |
| 30 | Ex. 30 | 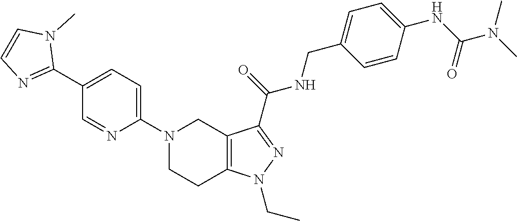 [SMILES: CCn1nc(C(=O)NCc2ccc(NC(=O)N(C)C)cc2)c2c1CCN(c1ccc(-c3nccn3C)cn1)C2] ¹H NMR (400 MHz, CD₃OD) δ 8.37 (d, 1H), 7.80 (dd, 1H), 7.36-7.31 (m, 2H), 7.30- 7.26 (m, 2H), 7.15 (d, 1H), 7.01 (d, 1H), 6.98 (d, 1H), 4.78 (s, 2H), 4.50 (s, 2H), 4.16- 4.04 (m, 4H), 3.74 (s, 3H), 3.01 (s, 6H), 2.85 (t, 2H), 1.41 (t, 3H). | N-(4-(3,3- dimethylureido)benzyl)- 1-ethyl-5-(5-(1-methyl- 1H-imidazol-2-yl)pyridin- 2-yl)-4,5,6,7-tetrahydro- 1H-pyrazolo[4,3- c]pyridine-3- carboxamide | 528.4 |
| 31 | Ex. 18 | 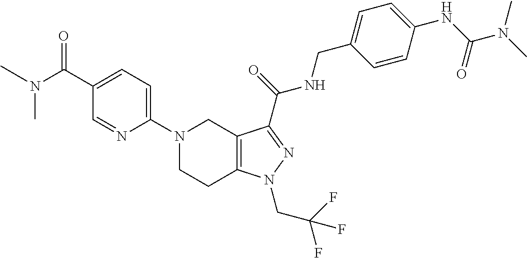 1H NMR* (600 MHz, (CD₃)₂SO) δ 8.52 (t, 1H), 8.24-8.21 (m, 2H), 7.66 (dd, 1H), 7.41-7.36 (m, 2H), 7.18-7.15 (m, 2H), 6.94 (d, 1H), 5.13 (q, 2H), 4.77 (s, 2H), 4.34 (d, 2H), 3.96 (t, 2H), 2.97 (s, 6H), 2.90 (s, 6H), 2.84 (t, 2H). | 5-(5- (dimethylcarbamoyl) pyridin-2-yl)-N-(4-(3,3- dimethylureido)benzyl)- 1-(2,2,2-trifluoroethyl)- 4,5,6,7-tetrahydro-1H- pyrazolo[4,3-c]pyridine- 3-carboxamide | 573.4 |
| 32 | Ex. 5 | 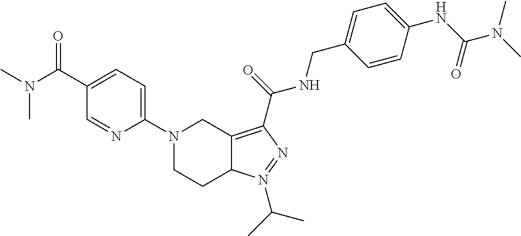 ¹H NMR (400 MHz, CD₃OD) δ 8.30-8.28 (m, 1H), 7.71 (dd, 1H), 7.35-7.31 (m, 2H), 7.28- 7.24 (m, 2H), 6.93 (d, 1H), 5.04 (s, 2H), 4.48 (s, 2H), 4.19-4.08 (m, 4H), 3.17-3.04 (m, 7H), 2.99 (s, 6H), 1.28 (d, 6H). | 7-(5- (dimethylcarbamoyl) pyridin-2-yl)-N-(4-(3,3- dimethylureido)benzyl)- 3-isopropyl-5,6,7,8- tetrahydroimidazo[1,5- a]pyrazine-1- carboxamide | 533.4 |
| 33 | Ex. 5 | 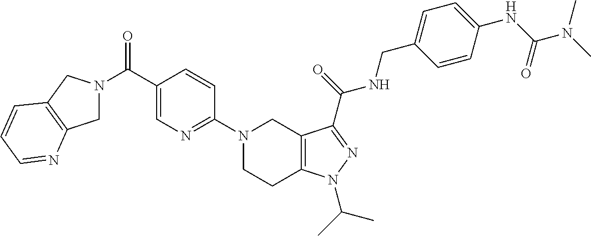 [SMILES: CC(C)n1nc(C(=O)NCc2ccc(NC(=O)N(C)C)cc2)c2c1CCN(c1ccc(C(=O)N3Cc4cccnc4C3)cn1)C2] ¹H NMR (400 MHz, (CD₃)₂SO) δ 8.54- 8.44 (m, 2H), 8.34 (t, 1H), 8.23 (s, 1H), 7.94- 7.67 (m, 2H), 7.42-7.37 (m, 2H), 7.32 (br s, 1H), 7.21-7.16 (m, 2H), 6.94 (d, 1H), 5.01-4.88 (m, 3H), 4.84-4.72 (m, 3H), 4.56-4.45 (m, 1H), 4.36 (d, 2H), 3.99 (t, 2H), 2.91 (s, 6H), 2.85 (t, 2H), 1.41 (d, 6H). | 5-(5-(6,7-dihydro-5H- pyrrolo[3,4-b]pyridine-6- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 608.4 |
| 34 | Ex. 34 | 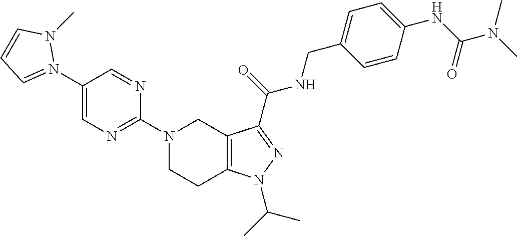 ¹H NMR (400 MHz, (CD₃)₂SO) δ 8.69 (s, 2H), 8.34 (t, 1H), 8.23 (s, 1H), 7.42-7.36 (m, 2H), 7.24 (d, 1H), 7.19-7.13 (m, 2H), 6.95 (d, 1H), 4.96 (s, 2H), 4.53-4.43 (m, 1H), 4.34 (d, 2H), 4.14 (t, 2H), 3.71 (s, 3H), 2.90 (s, 6H), 2.83 (t, 2H), 1.39 (d, 6H). | N-(4-(3,3- dimethylureido)benzyl)- 1-isopropyl-5-(5-(1- methyl-1H-imidazol-2- yl)pyrimidin-2-yl)- 4,5,6,7-tetrahydro-1H- pyrazolo[4,3-c]pyridine- 3-carboxamide | 543.5 |
| 35 | Ex. 35 | 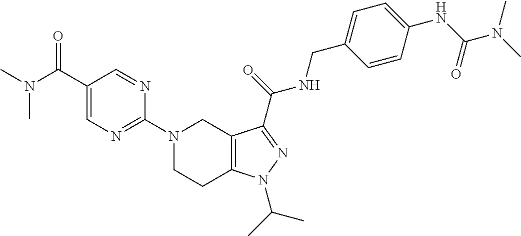 1H NMR (400 MHz, (CD₃)₂SO) δ 8.51 (s, 2H), 8.34 (t, 1H), 8.23 (s, 1H), 7.41-7.35 (m, 2H), 7.16 (d, 2H), 4.94 (s, 2H), 4.52- 4.42 (m, 1H), 4.33 (d, 2H), 4.12 (t, 2H), 2.97 (br s, 6H), 2.90 (s, 6H), 2.81 (t, 2H), 1.38 (d, 6H). | 5-(5- (dimethylcarbamoyl) pyrimidin-2-yl)-N-(4-(3,3- dimethylureido)benzyl)- 1-isopropyl-4,5,6,7- tetrahydro-1H- pyrazolo[4,3-c]pyridine- 3-carboxamide | 534.5 |
| 36 | Ex. 36 | 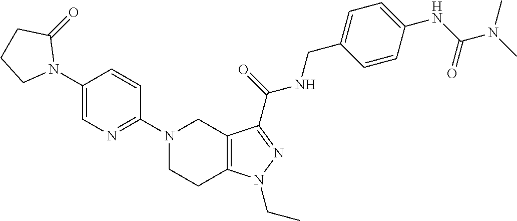 [SMILES: CCn1nc(C(=O)NCc2ccc(NC(=O)N(C)C)cc2)c2c1CCN(c1ccc(N3CCCC3=O)cn1)C2] ¹H NMR (400 MHz, (CD₃)₂SO) δ 8.42 (t, 1H), 8.30 (d, 1H), 8.23 (s, 1H), 7.90 (dd, 1H), 7.43-7.36 (m, 2H), 7.18 (d, 2H), 6.92 (d, 1H), 4.67 (s, 2H), 4.34 (d, 2H), 4.08 (q, 2H), 3.88 (t, 2H), 3.78 (t, 2H), 2.92 (s, 6H), 2.80 (t, 2H), 2.44 (t, 2H), 2.14-2.01 (m, 2H), 1.33 (t, 3H). | N-(4-(3,3- dimethylureido)benzyl)- 1-ethyl-5-(5-(2- oxopyrrolidin-1- yl)pyridin-2-yl)-4,5,6,7- tetrahydro-1H- pyrazolo[4,3-c]pyridine- 3-carboxamide | 531.5 |
| 37 | Ex. 37 | 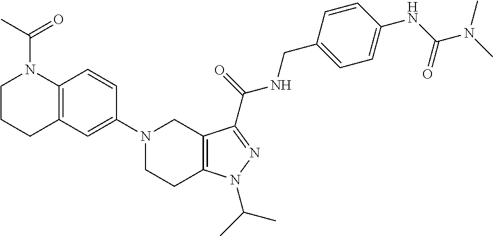 [SMILES: CC(=O)N1CCCc2cc(N3CCc4c(c(C(=O)NCc5ccc(NC(=O)N(C)C)cc5)nn4C(C)C)C3)ccc21] ¹H NMR (400 MHz, CD₃OD) δ 7.36-7.31 (m, 2H), 7.30-7.25 (m, 2H), 7.08 (br s, 1H), 6.90 (d, 2H), 4.54-4.46 (m, 3H), 4.40 (s, 2H), 3.73 (t, 2H), 3.61 (t, 2H), 3.00 (s, 6H), 2.85 (t, 2H), 2.72-2.63 (m, 2H), 2.16 (br s, 3H), 1.92 (br s, 2H), 1.46 (d, 6H). | 5-(1-acetyl-1,2,3,4- tetrahydroquinolin-6-yl)- N-(4-(3,3- dimethylureido)benzyl)- 1-isopropyl-4,5,6,7- tetrahydro-1H- pyrazolo[4,3-c]pyridine- 3-carboxamide | 558.5 |
| 38 | Ex. 1 | 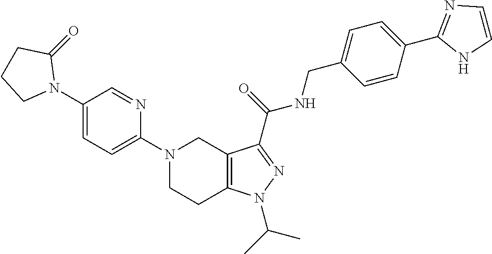 [SMILES: CC(C)n1nc(C(=O)NCc2ccc(-c3ncc[nH]3)cc2)c2c1CCN(c1ccc(N3CCCC3=O)cn1)C2] ¹H NMR (400 MHz, (CD₃)₂SO) δ 12.44 (br s, 1H), 8.47 (t, 1H), 8.30 (d, 1H), 7.93- 7.85 (m, 3H), 7.38 (d, 2H), 7.22 (br s, 1H), 7.00 (br s, 1H), 6.91 (d, 1H), 4.66 (s, 2H), 4.56-4.41 (m, 3H), 3.88 (t, 2H), 3.77 (t, 2H), 2.82 (t, 2H), 2.44 (t, 2H), 2.14-1.99 (m, 2H), 1.41 (d, 6H). | N-(4-(1H-imidazol-2- yl)benzyl)-1-isopropyl-5- (5-(2-oxopyrrolidin-1- yl)pyridin-2-yl)-4,5,6,7- tetrahydro-1H- pyrazolo[4,3-c]pyridine- 3-carboxamide | 525.3 |
| 39 | Ex. 39 | 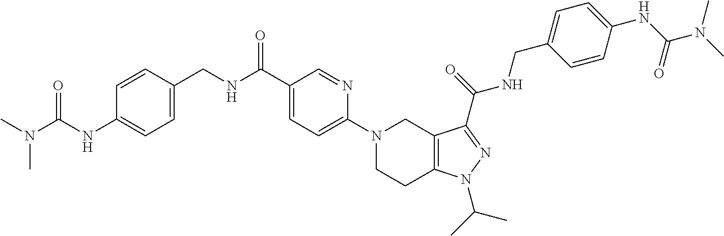 ¹H NMR (400 MHz, (CD₃)₂SO) δ 8.75 (t, 1H), 8.65 (d, 1H), 8.34 (t, 1H), 8.23 (d, 2H), 8.01 (dd, 1H), 7.41-7.35 (m, 4H), 7.20- 7.12 (m, 4H), 6.90 (d, 1H), 4.76 (s, 2H), 4.54-4.42 (m, 1H), 4.35 (dd, 4H), 3.97 (t, 2H), 2.81 (t, 2H), 1.39 (d, 6H). | N-(4-(3,3- dimethylureido)benzyl)- 5-(5-((4-(3,3- dimethylureido)benzyl) carbamoyl)pyridin-2-yl)-1- isopropyl-4,5,6,7- tetrahydro-1H- pyrazolo[4,3-c]pyridine- 3-carboxamide | 681.5 |
| 40 | Ex 5 | 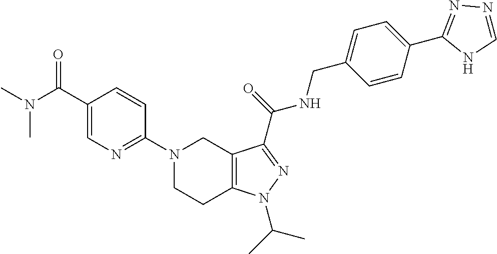 [SMILES: CC(C)n1nc(C(=O)NCc2ccc(-c3nnc[nH]3)cc2)c2c1CCN(c1ccc(C(=O)N(C)C)cn1)C2] ¹H NMR* (400 MHz, CD₃OD) δ 8.61-8.47 (m, 1H), 8.31-8.25 (m, 1H), 8.04-7.90 (m, 2H), 7.67 (dd, 1H), 7.54-7.43 (m, 2H), 6.89 (d, 1H), 4.78 (s, 2H), 4.64-4.59 (m, 2H), 4.57-4.49 (m, 1H), 4.08 (t, 2H), 3.08 (s, 6H), 2.87 (t, 2H), 1.52-1.45 (m, 6H). | N-(4-(4H-1,2,4-triazol-3- yl)benzyl)-5-(5- (dimethylcarbamoyl) pyridin-2-yl)-1-isopropyl- 4,5,6,7-tetrahydro-1H- pyrazolo[4,3-c]pyridine- 3-carboxamide | 514.3 |
| 41 | Ex. 41 | 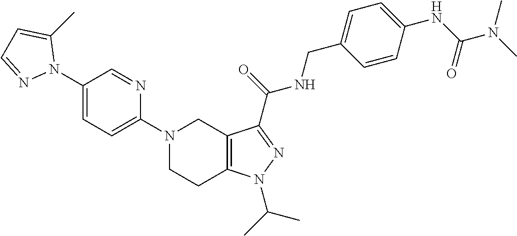 [SMILES: Cc1ccnn1-c1ccc(N2CCc3c(c(C(=O)NCc4ccc(NC(=O)N(C)C)cc4)nn3C(C)C)C2)nc1] ¹H NMR (400 MHz, CDCl₃) δ 8.22 (d, 1H), 7.58-7.53 (m, 2H), 7.36 (d, 2H), 7.30 (d, 2H), 7.15 (t, 1H), 6.86 (d, 1H), 6.31 (s, 1H), 6.17 (s, 1H), 4.76 (s, 2H), 4.56 (d, 2H), 4.43- 4.34 (m, 1H), 4.15 (t, 2H), 3.03 (s, 6H), 2.82 (t, 2H), 2.29 (s, 3H), 1.45 (d, 6H). | N-(4-(3,3- dimethylureido)benzyl)- 1-isopropyl-5-(5-(5- methyl-1H-pyrazol-1- yl)pyridin-2-yl)-4,5,6,7- tetrahydro-1H- pyrazolo[4,3-c]pyridine- 3-carboxamide | 542.4 |
| 42 | Ex. 42 | 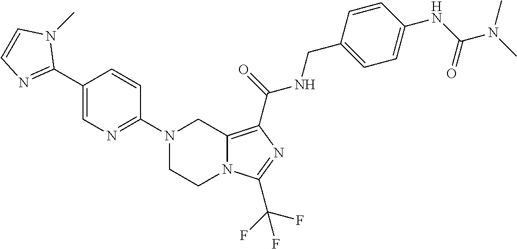 [SMILES: CN(C)C(=O)Nc1ccc(CNC(=O)c2nc(C(F)(F)F)n3c2CN(c2ccc(-c4nccn4C)cn2)CC3)cc1] ¹H NMR (400 MHz, CD₃OD) δ 8.43 (d, 1H), 7.86 (dd, 1H), 7.35-7.30 (m, 2H), 7.29- 7.24 (m, 2H), 7.16 (d, 1H), 7.07-7.00 (m, 2H), 5.15 (s, 2H), 4.49 (s, 2H), 4.34 (t, 2H), 4.21 (t, 2H), 3.74 (s, 3H), 3.00 (s, 6H). | N-(4-(3,3- dimethylureido)benzyl)- 7-(5-(1-methyl-1H- imidazol-2-yl)pyridin-2- yl)-3-(trifluoromethyl)- 5,6,7,8- tetrahydroimidazo[1,5- a]pyrazine-1- carboxamide | 568.3 |
| 43 | Ex. 43 | 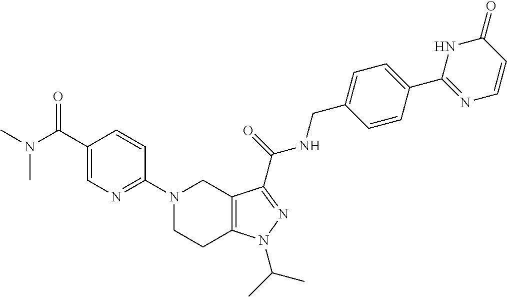 [SMILES: CC(C)n1nc(C(=O)NCc2ccc(-c3nccc(=O)[nH]3)cc2)c2c1CCN(c1ccc(C(=O)N(C)C)cn1)C2] ¹H NMR (400 MHz, (CD₃)₂SO) δ 8.57 (t, 1H), 8.23 (d, 1H), 8.09-8.02 (m, 3H), 7.63 (dd, 1H), 7.43 (d, 2H), 6.88 (d, 1H), 6.29 (d, 1H), 4.74 (s, 2H), 4.57-4.41 (m, 3H), 3.95 (t, 2H), 2.96 (s, 6H), 2.82 (t, 2H), 1.40 (d, 6H). | 5-(5- (dimethylcarbamoyl) pyridin-2-yl)-1-isopropyl-N- (4-(6-oxo-1,6- dihydropyrimidin-2- yl)benzyl)-4,5,6,7- tetrahydro-1H- pyrazolo[4,3-c]pyridine- 3-carboxamide | 541.3 |
| 44 | Ex 43 | 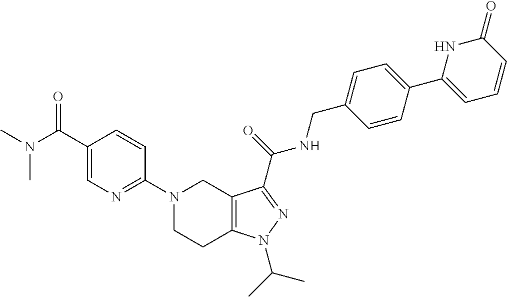 [SMILES: CC(C)n1nc(C(=O)NCc2ccc(-c3cccc(=O)[nH]3)cc2)c2c1CCN(c1ccc(C(=O)N(C)C)cn1)C2] ¹H NMR (400 MHz, (CD₃)₂SO) δ 11.54 (br s, 1H), 8.53 (q, 1H), 8.24-8.19 (m, 1H), 7.74-7.66 (m, 2H), 7.65-7.58 (m, 1H), 7.50 (q, 1H), 7.42-7.35 (m, 2H), 6.87 (t, 1H), 6.64-6.56 (m, 1H), 6.34 (t, 1H), 4.77- 4.69 (m, 2H), 4.55-4.40 (m, 3H), 3.98- 3.89 (m, 2H), 3.00-2.89 (m, 6H), 2.84- 2.76 (m, 2H), 1.42-1.35 (m, 6H). | 5-(5- (dimethylcarbamoyl) pyridin-2-yl)-1-isopropyl-N- (4-(6-oxo-1,6- dihydropyridin-2- yl)benzyl)-4,5,6,7- tetrahydro-1H- pyrazolo[4,3-c]pyridine- 3-carboxamide | 540.5 |
| 45 | Ex. 45 | 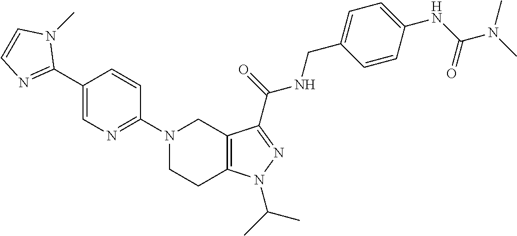 [SMILES: CC(C)n1nc(C(=O)NCc2ccc(NC(=O)N(C)C)cc2)c2c1CCN(c1ccc(-c3nccn3C)cn1)C2] ¹H NMR (600 MHz, (CD₃)₂SO) δ 8.44 (d, 1H), 8.34 (t, 1H), 8.23 (s, 1H), 7.86 (dd, 1H), 7.42-7.37 (m, 2H), 7.24 (br s, 1H), 7.20-7.16 (m, 2H), 6.98 (d, 2H), 4.77 (s, 2H), 4.55-4.47 (m, 1H), 4.35 (d, 2H), 3.97 (t, 2H), 3.72 (s, 3H) 2.91 (s, 6H), 2.85 (t, 2H), 1.41 (d, 6H). | N-(4-(3,3- dimethylureido)benzyl)- 1-isopropyl-5-(5-(1- methyl-1H-imidazol-2- yl)pyridin-2-yl)-4,5,6,7- tetrahydro-1H- pyrazolo[4,3-c]pyridine- 3-carboxamide | 542.3 |
| 46 | Ex. 12 | 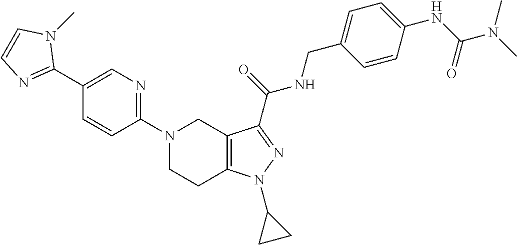 [SMILES: CN(C)C(=O)Nc1ccc(CNC(=O)c2nn(C3CC3)c3c2CN(c2ccc(-c4nccn4C)cn2)CC3)cc1] | 1-cyclopropyl-N-(4-(3,3- dimethylureido)benzyl)- 5-(5-(1-methyl-1H- imidazol-2-yl)pyridin-2- yl)-4,5,6,7-tetrahydro- 1H-pyrazolo[4,3- c]pyridine-3- carboxamide | 540.5 |
| 47 | Ex. 1 | 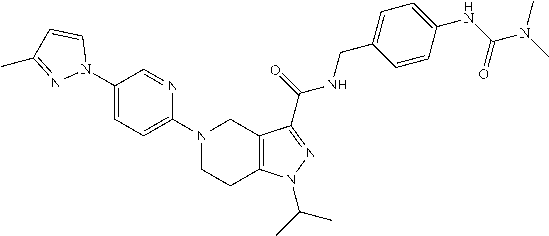 | N-(4-(3,3- dimethylureido)benzyl)- 1-isopropyl-5-(5-(3- methyl-1H-pyrazol-1- yl)pyridin-2-yl)-4,5,6,7- tetrahydro-1H- pyrazolo[4,3-c]pyridine- 3-carboxamide | 542.4 |
| 48 | Ex. 5 | 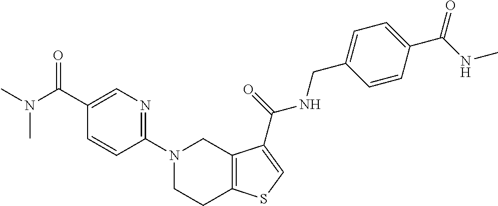 ¹H NMR (400 MHz, CD₃OD) δ 8.27 (d, 1H), 7.85 (s, 1H), 7.82-7.78 (m, 2H), 7.67 (dd, 1H), 7.46 (d, 2H), 6.88 (d, 1H), 4.86-4.84 (m, 2H), 4.59 (s, 2H), 4.05 (t, 2H), 3.09 (s, 6H), 2.95 (t, 2H), 2.91 (s, 3H). | 5-(5- (dimethylcarbamoyl) pyridin-2-yl)-N-(4- (methylcarbamoyl)benzyl)- 4,5,6,7- tetrahydrothieno[3,2- c]pyridine-3- carboxamide | 478.2 |
| 49 | Ex. 6 | 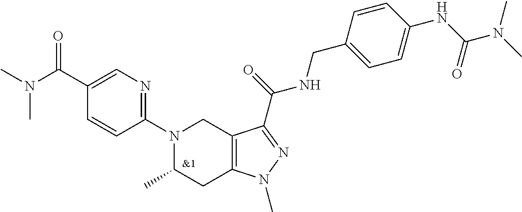 ¹H NMR (400 MHz, CDCl₃) δ 8.35-8.32 (m, 1H), 7.63 (d, 1H), 7.35 (d, 2H), 7.28 (d, 2H), 7.10 (t, 1H), 6.74 (d, 1H), 6.34 (s, 1H), 5.73-5.63 (m, 1H), 4.97 (d, 1H), 4.61- 4.49 (m, 2H), 4.33 (d, 1H), 3.75 (s, 3H), 3.08 (s, 6H), 3.04-2.98 (m, 7H), 2.54 (d, 1H), 1.13 (d, 3H). | rac-(R)-5-(5- (dimethylcarbamoyl) pyridin-2-yl)-N-(4-(3,3- dimethylureido)benzyl)- 1,6-dimethyl-4,5,6,7- tetrahydro-1H- pyrazolo[4,3-c]pyridine- 3-carboxamide | 519.2 |
| 50^{∧} | Ex. 113 | 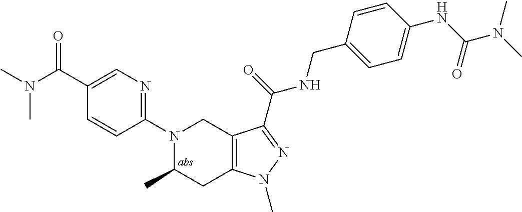 ¹H NMR (400 MHz, (CD₃)₂SO) δ 8.50 (t, 1H), 8.28-8.20 (m, 2H), 7.65 (dd, 1H), 7.41-7.34 (m, 2H), 7.20-7.13 (m, 2H), 6.85 (d, 1H), 5.33-5.09 (m, 2H), 4.38- 4.25 (m, 2H), 4.08 (d, 1H), 3.82-3.71 (m, 3H), 3.02-2.95 (m, 6H), 2.93-2.86 (m, 7H), 2.78-2.65 (m, 1H), 1.07 (d, 3H). | (R)-5-(5- (dimethylcarbamoyl) pyridin-2-yl)-N-(4-(3,3- dimethylureido)benzyl)- 1,6-dimethyl-4,5,6,7- tetrahydro-1H- pyrazolo[4,3-c]pyridine- 3-carboxamide | 519.3 |
| 51^{∧} | Ex. 113 | 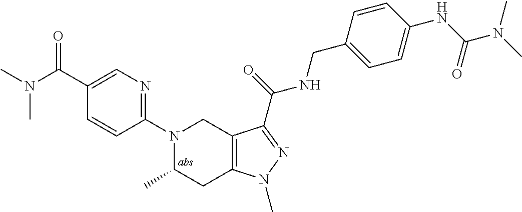 ¹H NMR (400 MHz, (CD₃)₂SO) δ 8.54- 8.48 (m, 1H), 8.28-8.21 (m, 2H), 7.66 (dd, 1H), 7.41-7.35 (m, 2H), 7.20-7.14 (m, 2H), 6.86 (d, 1H), 5.31-5.10 (m, 2H), 4.33 (d, 2H), 4.09 (d, 1H), 3.78 (s, 3H), 3.01- 2.95 (m, 6H), 2.94-2.88 (m, 7H), 2.77- 2.69 (m, 1H), 1.07 (d, 3H). | (S)-5-(5- (dimethylcarbamoyl) pyridin-2-yl)-N-(4-(3,3- dimethylureido)benzyl)- 1,6-dimethyl-4,5,6,7- tetrahydro-1H- pyrazolo[4,3-c]pyridine- 3-carboxamide | 519.4 |
| 52 | Ex. 5 | 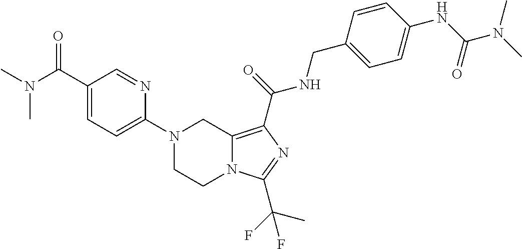 ¹H NMR (400 MHz, CD₃OD) δ 8.30 (d, 1H), 7.71 (dd, 1H), 7.34-7.28 (m, 2H), 7.26- 7.21 (m, 2H), 6.94 (d, 1H), 5.10 (s, 2H), 4.47 (s, 2H), 4.38-4.29 (m, 2H), 4.21- 4.12 (m, 2H), 3.07 (s, 6H), 2.98 (s, 6H), 2.17-2.01 (m, 3H). | 3-(1,1-difluoroethyl)-7- (5- (dimethylcarbamoyl) pyridin-2-yl)-N-(4-(3,3- dimethylureido)benzyl)- 5,6,7,8- tetrahydroimidazo[1,5- a]pyrazine-1- carboxamide | 555.5 |
| 53^{∧} | Ex. 5 | 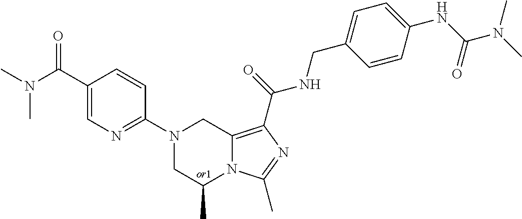 ¹H NMR (400 MHz, CD₃OD) δ 8.31 (d, 1H), 7.74 (dd, 1H), 7.36-7.31 (m, 2H), 7.28- 7.24 (m, 2H), 6.94 (d, 1H), 5.39 (d, 1H), 4.85-4.80 (m, 1H), 4.58-4.53 (m, 2H), 4.49 (s, 2H), 3.50-3.31 (m, 1H), 3.10 (s, 6H), 3.00 (s, 6H), 2.41 (s, 3H), 1.40 (d, 3H). | rel-(R or S)-7-(5- (dimethylcarbamoyl) pyridin-2-yl)-N-(4-(3,3- dimethylureido)benzyl)- 3,5-dimethyl-5,6,7,8- tetrahydroimidazo[1,5- a]pyrazine-1- carboxamide | 519.5 |
| 54^{∧} | Ex. 5 | 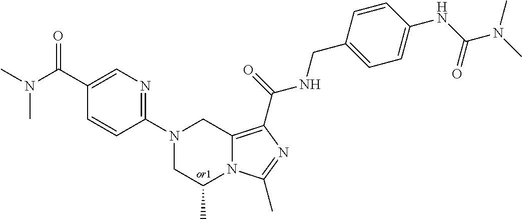 ¹H NMR (400 MHz, CD₃OD) δ 8.31 (d, 1H), 7.74 (dd, 1H), 7.36-7.31 (m, 2H), 7.28- 7.24 (m, 2H), 6.94 (d, 1H), 5.39 (d, 1H), 4.85-4.80 (m, 1H), 4.58-4.53 (m, 2H), 4.49 (s, 2H), 3.50-3.31 (m, 1H), 3.10 (s, 6H), 3.00 (s, 6H), 2.41 (s, 3H), 1.40 (d, 3H). | rel-(R or S)-7-(5- (dimethylcarbamoyl) pyridin-2-yl)-N-(4-(3,3- dimethylureido)benzyl)- 3,5-dimethyl-5,6,7,8- tetrahydroimidazo[1,5- a]pyrazine-1- carboxamide | 519.5 |
| 55 | Ex. 113 | 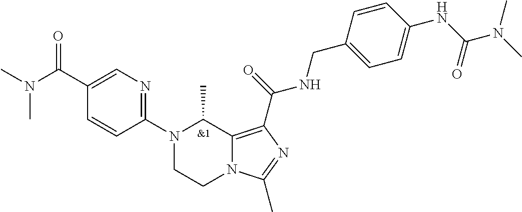 | rac-(R)-5-(5- (dimethylcarbamoyl) pyridin-2-yl)-N-(4-(3,3- dimethylureido)benzyl)- 1,4-dimethyl-4,5,6,7- tetrahydro-1H- pyrazolo[4,3-c]pyridine- 3-carboxamide | 519.5 |
| 56 | Ex. 1^{a} | 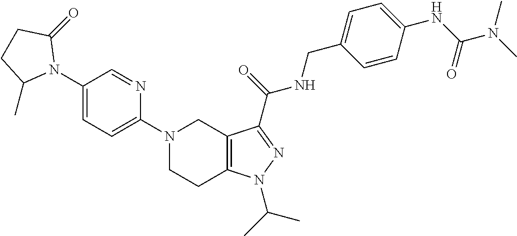 [SMILES: CC1CCC(=O)N1c1ccc(N2CCc3c(c(C(=O)NCc4ccc(NC(=O)N(C)C)cc4)nn3C(C)C)C2)nc1] | N-(4-(3,3- dimethylureido)benzyl)- 1-isopropyl-5-(5-(2- methyl-5-oxopyrrolidin- 1-yl)pyridin-2-yl)-4,5,6,7- tetrahydro-1H- pyrazolo[4,3-c]pyridine- 3-carboxamide | 559.4 |
| 57 | Ex. 43 | 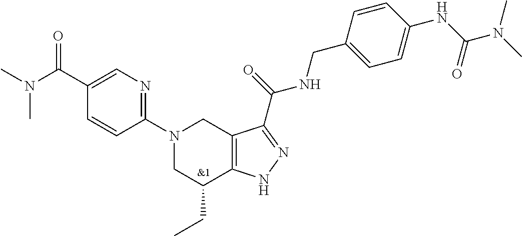 ¹H NMR (400 MHz, (CD₃)₂SO) δ 13.07 (s, 1H), 8.58-8.50 (m, 1H), 8.25-8.21 (m, 2H), 7.64 (dd, 1H), 7.41-7.35 (m, 2H), 7.19-7.14 (m, 2H), 6.85 (d, 1H), 4.80- 4.58 (m, 2H), 4.42-4.25 (m, 2H), 4.04- 3.89 (m, 1H), 3.81-3.67 (m, 1H), 2.97 (s, 6H), 2.93-2.88 (m, 7H), 1.85-1.70 (m, 1H), 1.60-1.46 (m, 1H), 1.00 (t, 3H). | rac-(R)-5-(5- (dimethylcarbamoyl) pyridin-2-yl)-N-(4-(3,3- dimethylureido)benzyl)- 7-ethyl-4,5,6,7- tetrahydro-1H- pyrazolo[4,3-c]pyridine- 3-carboxamide | 519.3 |
| 58^{∧} | Ex. 58 | 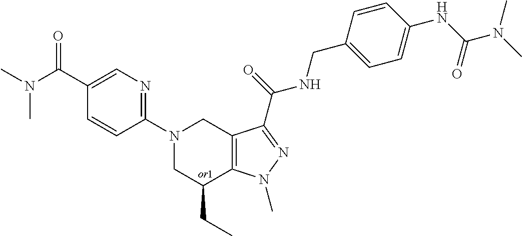 ¹H NMR (400 MHz, (CD₃)₂SO) δ 8.52 (t, 1H), 8.27-8.21 (m, 2H), 7.65 (dd, 1H), 7.40-7.34 (m, 2H), 7.18-7.11 (m, 2H), 6.83 (d, 1H), 5.00 (d, 1H), 4.76-4.69 (m, 1H), 4.42-4.16 (m, 3H), 3.81 (s, 3H), 3.24- 3.15 (m, 1H), 3.02-2.93 (m, 7H), 2.92- 2.87 (m, 6H), 1.66-1.53 (m, 1H), 1.50- 1.36 (m, 1H), 1.02 (t, 3H). | rel-(R or S)-5-(5- (dimethylcarbamoyl) pyridin-2-yl)-N-(4-(3,3- dimethylureido)benzyl)- 7-ethyl-1-methyl-4,5,6,7- tetrahydro-1H- pyrazolo[4,3-c]pyridine- 3-carboxamide | 533.4 |
| 59 | Ex. 42 | 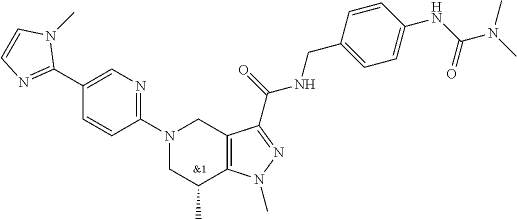 [SMILES: C[C@@H]1CN(c2ccc(-c3nccn3C)cn2)Cc2c(C(=O)NCc3ccc(NC(=O)N(C)C)cc3)nn(C)c21] ¹H NMR (400 MHz, (CD₃)₂SO) δ 8.50 (t, 1H), 8.43 (d, 1H), 8.23 (s, 1H), 7.84 (dd, 1H), 7.41-7.35 (m, 2H), 7.22-7.13 (m, 3H), 7.00-6.91 (m, 2H), 5.03 (d, 1H), 4.41- 4.22 (m, 4H), 3.83 (s, 3H), 3.71 (s, 3H), 3.50-3.39 (m, 1H), 3.25-3.17 (m, 1H), 2.90 (s, 6H), 1.23 (d, 3H). | rac-(R)-N-(4-(3,3- dimethylureido)benzyl)- 1,7-dimethyl-5-(5-(1- methyl-1H-imidazol-2- yl)pyridin-2-yl)-4,5,6,7- tetrahydro-1H- pyrazolo[4,3-c]pyridine- 3-carboxamide | 528.5 |
| 60 | Ex. 1^{a} | 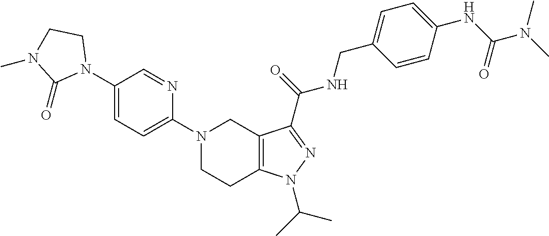 | N-(4-(3,3- dimethylureido)benzyl)- 1-isopropyl-5-(5-(3- methyl-2- oxoimidazolidin-1- yl)pyridin-2-yl)-4,5,6,7- tetrahydro-1H- pyrazolo[4,3-c]pyridine- 3-carboxamide | 560.4 |
| 61 | Ex. 1^{a} | 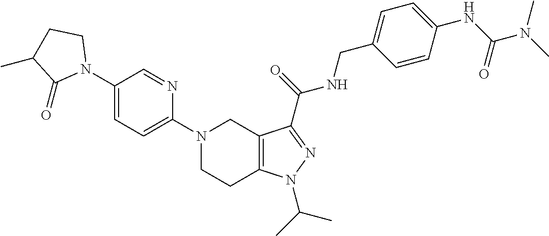 | N-(4-(3,3- dimethylureido)benzyl)- 1-isopropyl-5-(5-(3- methyl-2-oxopyrrolidin- 1-yl)pyridin-2-yl)-4,5,6,7- tetrahydro-1H- pyrazolo[4,3-c]pyridine- 3-carboxamide | 559.4 |
| 62 | Ex. 5 | 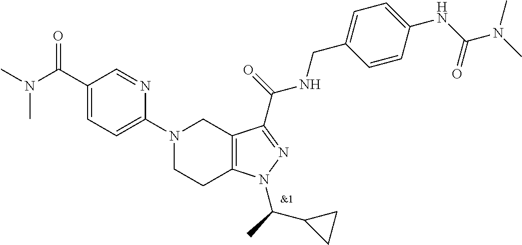 ¹H NMR (400 MHz, CD₃OD) δ 8.25 (d, 1H), 7.66 (dd, 1H), 7.35-7.30 (m, 2H), 7.29- 7.24 (m, 2H), 6.88 (d, 1H), 4.81-4.68 (m, 2H), 4.49 (s, 2H), 4.17-3.94 (m, 2H), 3.67- 3.56 (m, 1H), 3.14-3.03 (s, 6H), 2.99 (s, 6H), 2.81 (t, 2H), 1.55 (d, 3H), 1.44-1.32 (m, 1H), 0.69-0.57 (m, 1H), 0.49-0.37 (m, 1H), 0.35-0.20 (m, 2H). | rac-(R)-1-(1- cyclopropylethyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 559.3 |
| 63^{∧} | Ex.1^{a} | 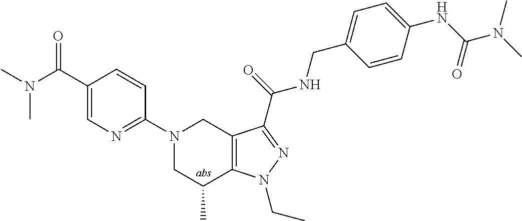 ¹H NMR (400 MHz, CD₃OD) δ 8.28-8.26 (m, 1H), 7.68 (dd, 1H), 7.36-7.31 (m, 2H), 7.29-7.23 (m, 2H), 6.89 (d, 1H), 5.04 (d, 1H), 4.64-4.56 (m, 1H), 4.49 (s, 2H), 4.36 (d, 1H), 4.14 (q, 2H), 3.43-3.32 (m, 1H), 3.26-3.15 (m, 1H), 3.09 (s, 6H), 3.00 (s, 6H), 1.46 (t, 3H), 1.28 (d, 3H). | (R)-5-(5- (dimethylcarbamoyl) pyridin-2-yl)-N-(4-(3,3- dimethylureido)benzyl)- 1-ethyl-7-methyl-4,5,6,7- tetrahydro-1H- pyrazolo[4,3-c]pyridine- 3-carboxamide | 533.4 |
| 64 | Ex. 19 | 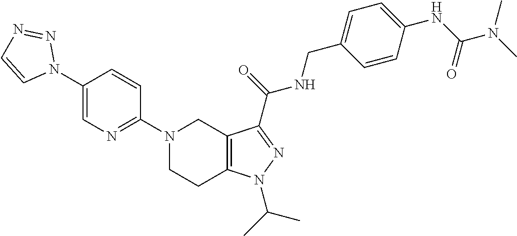 [SMILES: CC(C)n1nc(C(=O)NCc2ccc(NC(=O)N(C)C)cc2)c2c1CCN(c1ccc(-n3ccnn3)cn1)C2] ¹H NMR (400 MHz, (CD₃)₂SO) δ 8.68- 8.65 (m, 1H), 8.59 (d, 1H), 8.36 (t, 1H), 8.24 (s, 1H), 8.01 (dd, 1H), 7.95-7.91 (m, 1H), 7.41-7.36 (m, 2H), 7.20-7.15 (m, 2H), 7.08 (d, 1H), 4.77 (s, 2H), 4.56-4.43 (m, 1H), 4.35 (d, 2H), 3.97 (t, 2H), 2.90 (s, 6H), 2.88-2.80 (m, 2H), 1.39 (d, 6H). | 5-(5-(1H-1,2,3-triazol-1- yl)pyridin-2-yl)-N-(4-(3,3- dimethylureido)benzyl)- 1-isopropyl-4,5,6,7- tetrahydro-1H- pyrazolo[4,3-c]pyridine- 3-carboxamide | 529.3 |
| 65 | Ex. 1^{a} | 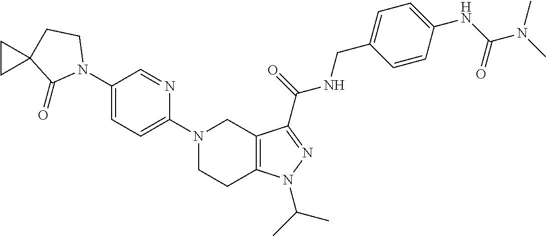 [SMILES: CC(C)n1nc(C(=O)NCc2ccc(NC(=O)N(C)C)cc2)c2c1CCN(c1ccc(N3CCC4(CC4)C3=O)cn1)C2] | N-(4-(3,3- dimethylureido)benzyl)- 1-isopropyl-5-(5-(4-oxo- 5-azaspiro[2.4]heptan-5- yl)pyridin-2-yl)-4,5,6,7- tetrahydro-1H- pyrazolo[4,3-c]pyridine- 3-carboxamide | 571.4 |
| 66 | Ex. 1^{a} | 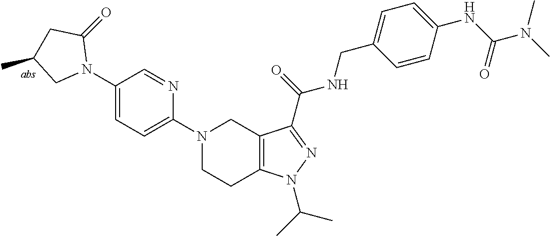 | (S)-N-(4-(3,3- dimethylureido)benzyl)- 1-isopropyl-5-(5-(4- methyl-2-oxopyrrolidin- 1-yl)pyridin-2-yl)-4,5,6,7- tetrahydro-1H- pyrazolo[4,3-c]pyridine- 3-carboxamide | 559.4 |
| 67 | Ex. 1^{a} | 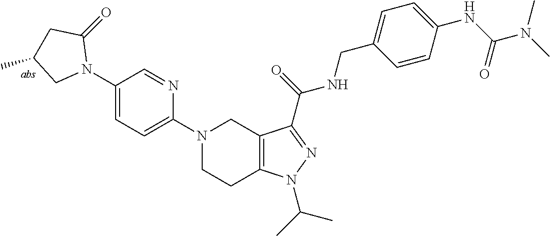 | (R)-N-(4-(3,3- dimethylureido)benzyl)- 1-isopropyl-5-(5-(4- methyl-2-oxopyrrolidin- 1-yl)pyridin-2-yl)-4,5,6,7- tetrahydro-1H- pyrazolo[4,3-c]pyridine- 3-carboxamide | 559.4 |
| 68 | Ex.5 | 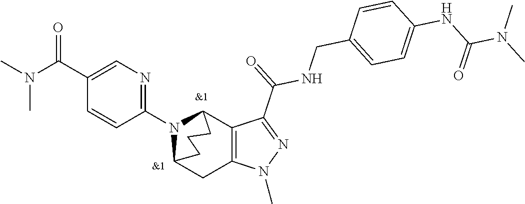 ¹H NMR* (400 MHz, CD₃OD) δ 8.38-8.31 (m, 1H), 8.24 (d, 1H), 7.61 (dd, 1H), 7.37- 7.31 (m, 2H), 7.30-7.24 (m, 2H), 6.87 (d, 1H), 5.90-5.83 (m, 1H), 5.26-5.16 (m, 1H), 4.49 (d, 2H), 3.75 (s, 3H), 3.17-3.03 (m, 7H), 3.01 (s, 6H), 2.65 (d, 1H), 1.97- 1.78 (m, 4H), 1.59-1.42 (m, 2H). | rac-(4R,8S)-10-(5- (dimethylcarbamoyl) pyridin-2-yl)-N-(4-(3,3- dimethylureido)benzyl)- 1-methyl-4,5,6,7,8,9- hexahydro-1H-4,8- epiminocycloocta[c] pyrazole-3-carboxamide | 545.3 |
| 69 | Ex. 1^{a} | 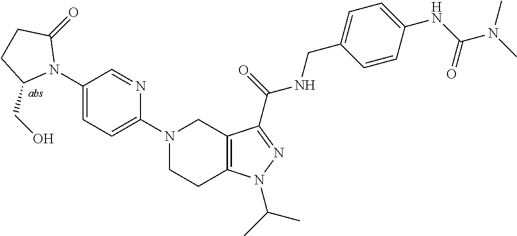 [SMILES: CC(C)n1nc(C(=O)NCc2ccc(NC(=O)N(C)C)cc2)c2c1CCN(c1ccc(N3C(=O)CC[C@H]3CO)cn1)C2] | (S)-N-(4-(3,3- dimethylureido)benzyl)- 5-(5-(2-(hydroxymethyl)- 5-oxopyrrolidin-1- yl)pyridin-2-yl)-1- isopropyl-4,5,6,7- tetrahydro-1H- pyrazolo[4,3-c]pyridine- 3-carboxamide | 575.4 |
| 70 | Ex. 87 | 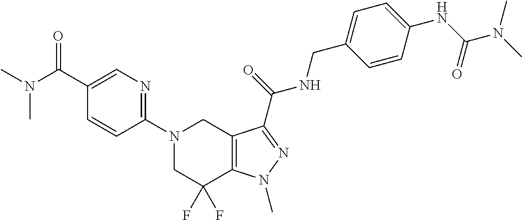 ¹H NMR (400 MHz, CD₃OD) δ 8.30 (d, 1H), 7.72 (dd, 1H), 7.36-7.31 (m, 2H), 7.29- 7.24 (m, 2H), 6.99 (d, 1H), 4.62-4.57 (m, 2H), 4.52-4.42 (m, 4H), 4.04 (s, 3H), 3.12- 3.03 (m, 6H), 3.01-2.95 (m, 6H). | 5-(5- (dimethylcarbamoyl) pyridin-2-yl)-N-(4-(3,3- dimethylureido)benzyl)- 7,7-difluoro-1-methyl- 4,5,6,7-tetrahydro-1H- pyrazolo[4,3-c]pyridine- 3-carboxamide | 541.2 |
| 71 | Ex. 43 | 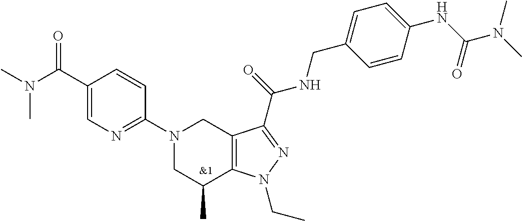 ¹H NMR (400 MHz, (CD₃)₂SO) δ 8.46 (t, 1H), 8.27-8.21 (m, 2H), 7.64 (dd, 1H), 7.40-7.35 (m, 2H), 7.19-7.14 (m, 2H), 6.89 (d, 1H), 5.05 (d, 1H), 4.41-4.23 (m, 4H), 4.19-4.04 (m, 2H), 3.28-3.16 (m, 2H), 2.98 (s, 6H), 2.93-2.87 (m, 6H), 1.38 (t, 3H), 1.20 (d, 3H). | rac-(R)-5-(5- (dimethylcarbamoyl) pyridin-2-yl)-N-(4-(3,3- dimethylureido)benzyl)- 1-ethyl-7-methyl-4,5,6,7- tetrahydro-1H- pyrazolo[4,3-c]pyridine- 3-carboxamide | 533.4 |
| 72 | Ex. 87 | 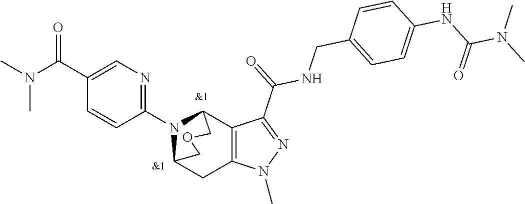 ¹H NMR (400 MHz, (CD₃)₂SO) δ 8.47 (t, 1H), 8.25-8.18 (m, 2H), 7.63 (dd, 1H), 7.40-7.36 (m, 2H), 7.18-7.12 (m, 2H), 6.83 (d, 1H), 5.73 (s, 1H), 4.80-4.71 (m, 1H), 4.42-4.22 (m, 2H), 3.98-3.92 (m, 1H), 3.83-3.64 (m, 6H), 3.11-3.01 (m, 1H), 2.94 (s, 6H), 2.91 (s, 6H), 2.84-2.75 (m, 1H). | rac-(4R,8S)-10-(5- (dimethylcarbamoyl) pyridin-2-yl)-N-(4-(3,3- dimethylureido)benzyl)- 1-methyl-1,4,5,7,8,9- hexahydro-4,8- epiminooxocino[5,4- c]pyrazole-3- carboxamide | 547.2 |
| 73 | Ex. 1^{a} | 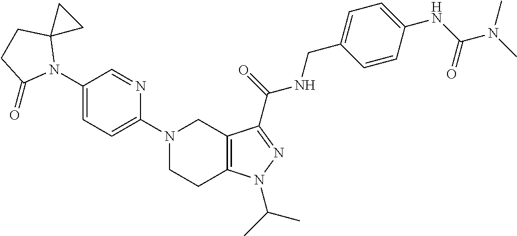 [SMILES: CC(C)n1nc(C(=O)NCc2ccc(NC(=O)N(C)C)cc2)c2c1CCN(c1ccc(N3C(=O)CCC34CC4)cn1)C2] | N-(4-(3,3- dimethylureido)benzyl)- 1-isopropyl-5-(5-(5-oxo- 4-azaspiro[2.4]heptan-4- yl)pyridin-2-yl)-4,5,6,7- tetrahydro-1H- pyrazolo[4,3-c]pyridine- 3-carboxamide | 571.5 |
| 74 | Ex.5 | 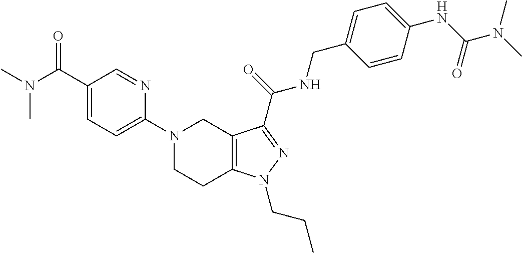 ¹H NMR (400 MHz, CD₃OD) δ 8.27-8.24 (m, 1H), 7.69-7.64 (m, 1H), 7.36-7.31 (m, 2H), 7.29-7.24 (m, 2H), 6.92-6.86 (m, 1H), 4.76 (s, 2H), 4.48 (s, 2H), 4.09- 4.00 (m, 4H), 3.08 (s, 6H), 3.01-2.99 (m, 6H), 2.86-2.79 (m, 2H), 1.89-1.78 (m, 2H), 0.92-0.85 (m, 3H). | 5-(5- (dimethylcarbamoyl) pyridin-2-yl)-N-(4-(3,3- dimethylureido)benzyl)- 1-propyl-4,5,6,7- tetrahydro-1H- pyrazolo[4,3-c]pyridine- 3-carboxamide | 533.2 |
| 75 | Ex. 1^{a} | 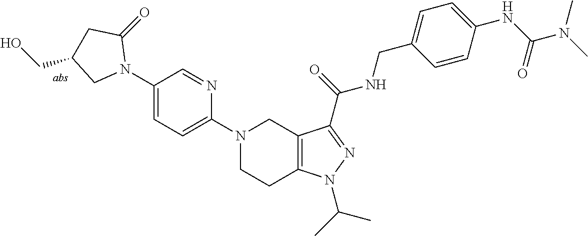 [SMILES: CC(C)n1nc(C(=O)NCc2ccc(NC(=O)N(C)C)cc2)c2c1CCN(c1ccc(N3C[C@H](CO)CC3=O)cn1)C2] | (R)-N-(4-(3,3- dimethylureido)benzyl)- 5-(5-(4-(hydroxymethyl)- 2-oxopyrrolidin-1- yl)pyridin-2-yl)-1- isopropyl-4,5,6,7- tetrahydro-1H- pyrazolo[4,3-c]pyridine- 3-carboxamide | 575.4 |
| 76 | Ex. 5 | 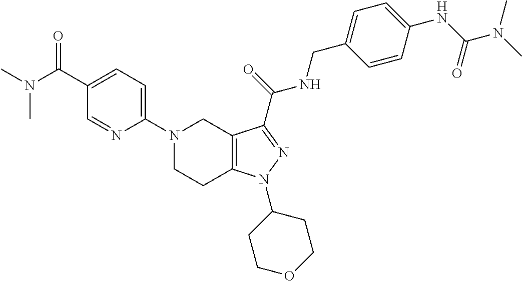 ¹H NMR (400 MHz, (CD₃)₂SO) δ 8.42 (t, 1H), 8.25-8.22 (m, 2H), 7.64 (dd, 1H), 7.42-7.35 (m, 2H), 7.20-7.13 (m, 2H), 6.89 (d, 1H), 4.75 (s, 2H), 4.46-4.30 (m, 3H), 4.00-3.90 (m, 4H), 3.45 (t, 2H), 2.97 (s, 6H), 2.90 (s, 6H), 2.87-2.81 (m, 2H), 2.13-1.98 (m, 2H), 1.86-1.78 (m, 2H). | 5-(5- (dimethylcarbamoyl) pyridin-2-yl)-N-(4-(3,3- dimethylureido)benzyl)- 1-(tetrahydro-2H-pyran- 4-yl)-4,5,6,7-tetrahydro- 1H-pyrazolo[4,3- c]pyridine-3- carboxamide | 575.5 |
| 77 | Ex. 77 | 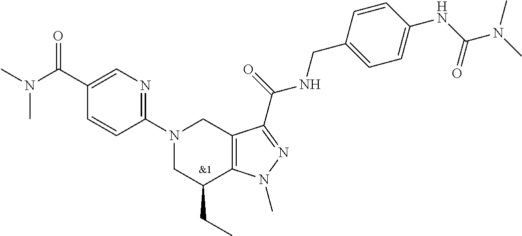 ¹H NMR (400 MHz, (CD₃)₂SO) δ 8.51 (t, 1H), 8.27-8.20 (m, 2H), 7.65 (dd, 1H), 7.41-7.35 (m, 2H), 7.18-7.12 (m, 2H), 6.83 (d, 1H), 5.01 (d, 1H), 4.73 (d, 1H), 4.40- 4.18 (m, 3H), 3.82 (s, 3H), 3.23-3.09 (m, 1H), 3.04-2.94 (m, 7H), 2.93-2.87 (m, 6H), 1.66-1.53 (m, 1H), 1.49-1.36 (m, 1H), 1.03 (t, 3H). | rac-(R)-5-(5- (dimethylcarbamoyl) pyridin-2-yl)-N-(4-(3,3- dimethylureido)benzyl)- 7-ethyl-1-methyl-4,5,6,7- tetrahydro-1H- pyrazolo[4,3-c]pyridine- 3-carboxamide | 533.4 |
| 78^{∧} | Ex. 78 | 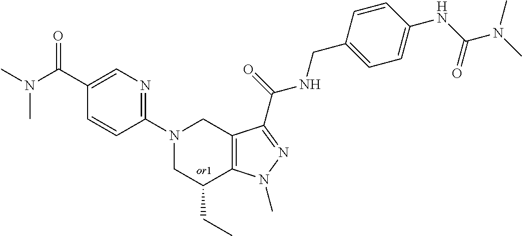 ¹H NMR (400 MHz, (CD₃)₂SO) δ 8.49 (t, 1H), 8.24-8.16 (m, 2H), 7.61 (dd, 1H), 7.36-7.31 (m, 2H), 7.16-7.09 (m, 2H), 6.80 (d, 1H), 4.97 (d, 1H), 4.69 (d, 1H), 4.35- 4.15 (m, 3H), 3.78 (s, 3H), 3.20-3.10 (m, 1H), 2.98-2.91 (m, 7H), 2.87 (m, 6H), 1.62- 1.49 (m, 1H), 1.46-1.32 (m, 1H), 0.99 (t, 3H). | rel-(R or S)-5-(5- (dimethylcarbamoyl) pyridin-2-yl)-N-(4-(3,3- dimethylureido)benzyl)- 7-ethyl-1-methyl-4,5,6,7- tetrahydro-1H- pyrazolo[4,3-c]pyridine- 3-carboxamide | 533.4 |
| 79 | Ex. 1^{a} | 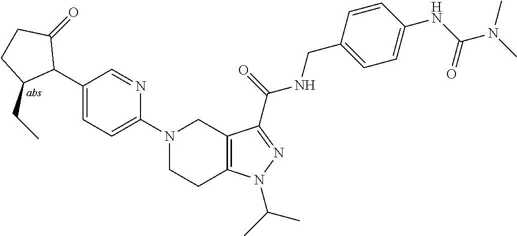 [SMILES: CC[C@H]1CCC(=O)C1c1ccc(N2CCc3c(c(C(=O)NCc4ccc(NC(=O)N(C)C)cc4)nn3C(C)C)C2)nc1] | (S)-N-(4-(3,3- dimethylureido)benzyl)- 5-(5-(2-ethyl-5- oxopyrrolidin-1- yl)pyridin-2-yl)-1- isopropyl-4,5,6,7- tetrahydro-1H- pyrazolo[4,3-c]pyridine- 3-carboxamide | 573.4 |
| 80 | Ex. 1^{a} | 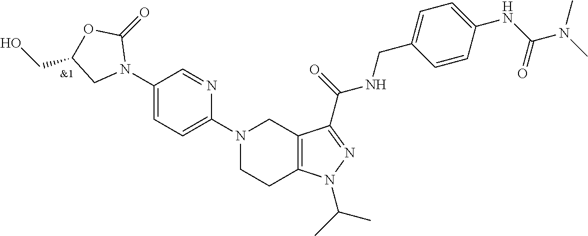 [SMILES: CC(C)n1nc(C(=O)NCc2ccc(NC(=O)N(C)C)cc2)c2c1CCN(c1ccc(N3C[C@H](CO)OC3=O)cn1)C2] | rac-(R)-N-(4-(3,3- dimethylureido)benzyl)- 5-(5-(5-(hydroxymethyl)- 2-oxooxazolidin-3- yl)pyridin-2-yl)-1- isopropyl-4,5,6,7- tetrahydro-1H- pyrazolo[4,3-c]pyridine- 3-carboxamide | 577.4 |
| 81 | Ex. 105 | 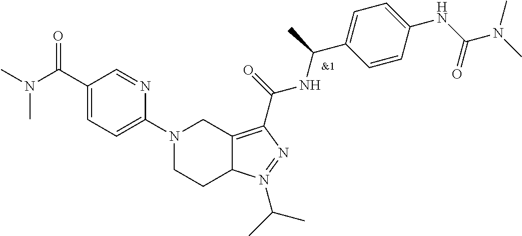 ¹H NMR (400 MHz, (CD₃)₂SO) δ 8.26- 8.20 (m, 2H), 7.99 (d, 1H), 7.62 (dd, 1H), 7.41-7.34 (m, 2H), 7.28-7.21 (m, 2H), 6.87 (d, 1H), 5.14-5.02 (m, 1H), 4.71 (s, 2H), 4.55-4.42 (m, 1H), 3.92 (d, 2H), 2.96 (s, 6H), 2.90 (s, 6H), 2.81 (t, 2H), 1.47 (d, 3H), 1.40 (dd, 6H). | rac-(R)-5-(5- (dimethylcarbamoyl) pyridin-2-yl)-N-(1-(4-(3,3- dimethylureido)phenyl) ethyl)-1-isopropyl-4,5,6,7- tetrahydro-1H- pyrazolo[4,3-c]pyridine- 3-carboxamide | 547.5 |
| 82^{∧} | Ex. 113 | 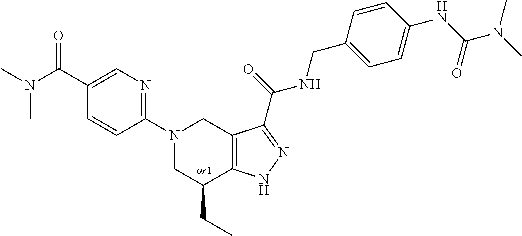 ¹H NMR (400 MHz, (CD₃)₂SO) δ 13.07- 13.02 (m, 1H), 8.57-8.48 (m, 1H), 8.23- 8.16 (m, 2H), 7.61 (dd, 1H), 7.38-7.30 (m, 2H), 7.16-7.10 (m, 2H), 6.82 (d, 1H), 4.75- 4.56 (m, 2H), 4.38-4.25 (m, 2H), 3.96- 3.89 (m, 1H), 3.79-3.70 (m, 1H), 2.99- 2.90 (m, 6H), 2.89-2.82 (m, 7H), 1.82- 1.69 (m, 1H), 1.58-1.43 (m, 1H), 0.97 (t, 3H). | rel-(R or S)-5-(5- (dimethylcarbamoyl) pyridin-2-yl)-N-(4-(3,3- dimethylureido)benzyl)- 7-ethyl-4,5,6,7- tetrahydro-1H- pyrazolo[4,3-c]pyridine- 3-carboxamide | 519.5 |
| 83^{∧} | Ex. 113 | 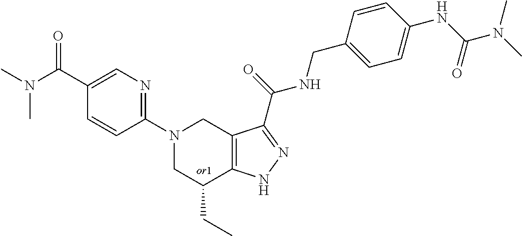 ¹H NMR (400 MHz, (CD₃)₂SO) δ 13.04 (s, 1H), 8.56-8.49 (m, 1H), 8.24-8.16 (m, 2H), 7.60 (dd, 1H), 7.39-7.31 (m, 2H), 7.16- 7.09 (m, 2H), 6.81 (d, 1H), 4.78-4.54 (m, 2H), 4.40-4.24 (m, 2H), 3.97-3.86 (m, 1H), 3.80-3.67 (m, 1H), 2.98-2.91 (m, 6H), 2.89-2.83 (m, 7H), 1.82-1.69 (m, 1H), 1.57-1.41 (m, 1H), 0.96 (t, 3H). | rel-(R or S)-5-(5- (dimethylcarbamoyl) pyridin-2-yl)-N-(4-(3,3- dimethylureido)benzyl)- 7-ethyl-4,5,6,7- tetrahydro-1H- pyrazolo[4,3-c]pyridine- 3-carboxamide | 519.5 |
| 84 | Ex. 1^{a} | 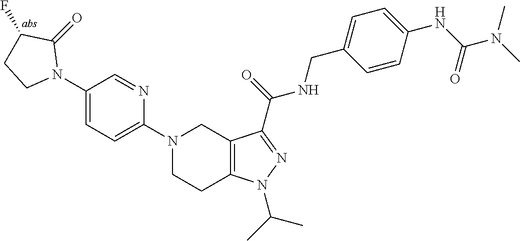 [SMILES: CC(C)n1nc(C(=O)NCc2ccc(NC(=O)N(C)C)cc2)c2c1CCN(c1ccc(N3CC[C@H](F)C3=O)cn1)C2] | (S)-N-(4-(3,3- dimethylureido)benzyl)- 5-(5-(3-fluoro-2- oxopyrrolidin-1- yl)pyridin-2-yl)-1- isopropyl-4,5,6,7- tetrahydro-1H- pyrazolo[4,3-c]pyridine- 3-carboxamide | 563.3 |
| 85 | Ex. 43 | 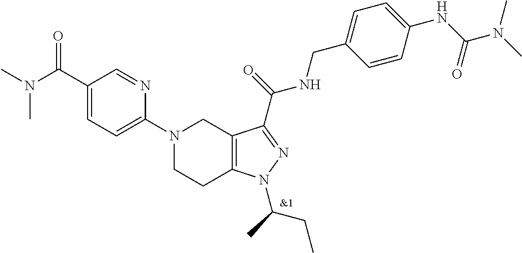 ¹H NMR (400 MHz, (CD₃)₂SO) δ 8.33 (t, 1H), 8.26-8.19 (m, 2H), 7.63 (dd, 1H), 7.42-7.34 (m, 2H), 7.21-7.14 (m, 2H), 6.88 (d, 1H), 4.80-4.68 (m, 2H), 4.41- 2.26 (m, 2H), 4.25-4.15 (m, 1H), 4.03- 3.84 (m, 2H), 2.96 (s, 6H), 2.90 (s, 6H), 2.83-2.75 (m, 2H), 1.93-1.63 (m, 2H), 1.38 (d, 3H), 0.69 (t, 3H). | rac-(R)-1-(sec-butyl)-5- (5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 547.3 |
| 86 | Ex. 5 | 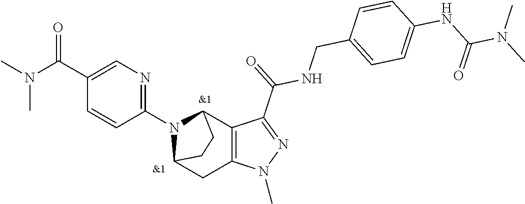 ¹H NMR (400 MHz, CDCl₃) δ 8.18 (d, 1H), 7.39-7.32 (m, 2H), 7.30-7.26 (m, 2H), 7.06-6.99 (m, 1H), 6.61-6.53 (m, 2H), 6.31 (s, 1H), 5.55-5.52 (m, 1H), 5.10- 5.00 (m, 1H), 4.62-4.44 (m, 2H), 3.59 (s, 3H), 3.32-3.19 (m, 1H), 3.06 (s, 3H), 3.03 (s, 6H), 2.85 (s, 3H), 2.46-2.27 (m, 3H), 2.14-2.03 (m, 1H), 1.72-1.63 (m, 1H). | rac-(4R,7S)-9-(5- (dimethylcarbamoyl) pyridin-2-yl)-N-(4-(3,3- dimethylureido)benzyl)- 1-methyl-1,4,5,6,7,8- hexahydro-4,7- epiminocyclohepta[c] pyrazole-3-carboxamide | 531.3 |
| 87^{∧} | Ex. 5 | 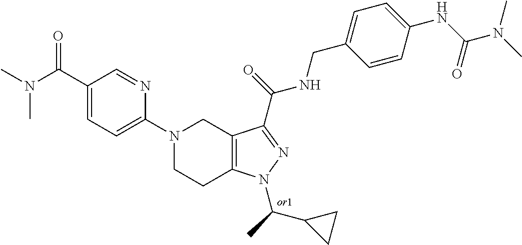 ¹H NMR (400 MHz, CD₃OD) δ 8.28 (d, 1H), 7.69 (dd, 1H), 7.39-7.33 (m, 2H), 7.32- 7.28 (m, 2H), 6.91 (d, 1H), 4.86-4.71 (m, 2H), 4.52 (s, 2H), 4.22-3.96 (m, 2H), 3.71- 3.58 (m, 1H), 3.10 (s, 6H), 3.02 (s, 6H), 2.84 (t, 2H), 1.58 (d, 3H), 1.46-1.35 (m, 1H), 0.72-0.61 (m, 1H), 0.52-0.42 (m, 1H), 0.39-0.25 (m, 2H). | rel-(R or S)-1-(1- cyclopropylethyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 559.3 |
| 88 | Ex. 1^{a} | 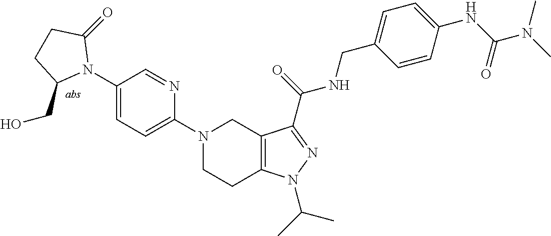 [SMILES: CC(C)n1nc(C(=O)NCc2ccc(NC(=O)N(C)C)cc2)c2c1CCN(c1ccc(N3C(=O)CC[C@@H]3CO)cn1)C2] | (R)-N-(4-(3,3- dimethylureido)benzyl)- 5-(5-(2-(hydroxymethyl)- 5-oxopyrrolidin-1- yl)pyridin-2-yl)-1- isopropyl-4,5,6,7- tetrahydro-1H- pyrazolo[4,3-c]pyridine- 3-carboxamide | 575.4 |
| 89 | Ex. 5 | 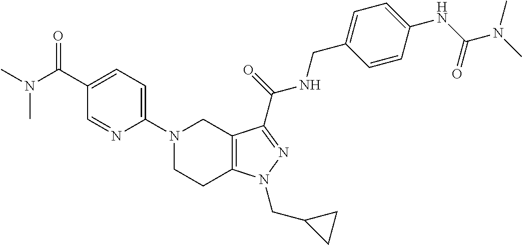 ¹H NMR (400 MHz, CD₃OD) δ 8.27-8.25 (m, 1H), 7.67 (dd, 1H), 7.36-7.30 (m, 2H), 7.29-7.24 (m, 2H), 6.89 (d, 1H), 4.79- 4.72 (m, 2H), 4.51-4.45 (m, 2H), 4.06 (t, 2H), 3.96 (d, 2H), 3.08 (s, 6H), 3.00 (s, 6H), 2.85 (t, 2H), 1.30-1.18 (m, 1H), 0.59- 0.50 (m, 2H), 0.40-0.33 (m, 2H). | 1-(cyclopropylmethyl)-5- (5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 545.4 |
| 90 | Ex. 5 | 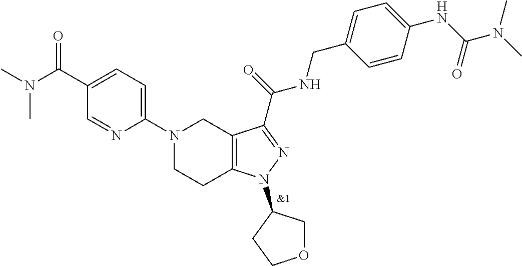 ¹H NMR (400 MHz, CD₃OD) δ 8.63 (t, 1H), 8.27 (d, 1H), 7.68 (dd, 1H), 7.37-7.31 (m, 2H), 7.30-7.25 (m, 2H), 6.90 (d, 1H), 5.07- 4.97 (m, 1H), 4.78 (s, 2H), 4.52-4.48 (m, 1H), 4.20-4.00 (m, 5H), 3.93-3.85 (m, 1H), 3.09 (s, 6H), 3.01 (s, 6H), 2.88 (t, 2H), 2.50-2.37 (m, 1H), 2.34-2.23 (m, 1H). | rac-(R)-5-(5- (dimethylcarbamoyl) pyridin-2-yl)-N-(4-(3,3- dimethylureido)benzyl)- 1-(tetrahydrofuran-3-yl)- 4,5,6,7-tetrahydro-1H- pyrazolo[4,3-c]pyridine- 3-carboxamide | 561.3 |
| 91 | Ex. 1^{a} | 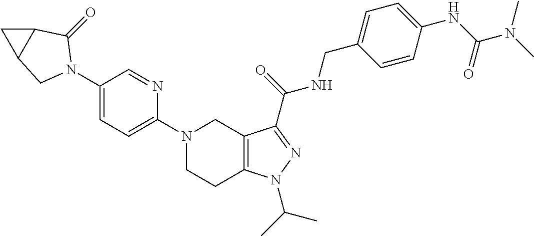 [SMILES: CC(C)n1nc(C(=O)NCc2ccc(NC(=O)N(C)C)cc2)c2c1CCN(c1ccc(N3CC4CC4C3=O)cn1)C2] | N-(4-(3,3- dimethylureido)benzyl)- 1-isopropyl-5-(5-(2-oxo- 3. azabicyclo[3.1.0]hexan- 3-yl)pyridin-2-yl)-4,5,6,7- tetrahydro-1H- pyrazolo[4,3-c]pyridine- 3-carboxamide | 557.4 |
| 92 | Ex. 43 | 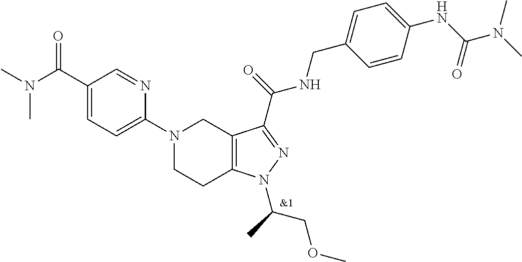 ¹H NMR (400 MHz, (CD₃)₂SO) δ 8.35 (t, 1H), 8.25-8.22 (m, 2H), 7.64 (dd, 1H), 7.42-7.36 (m, 2H), 7.20-7.14 (m, 2H), 6.88 (d, 1H), 4.87-4.63 (m, 2H), 4.55- 4.44 (m, 1H), 4.34 (d, 2H), 4.06-3.83 (m, 2H), 3.70-3.47 (m, 2H), 3.16 (s, 3H), 2.97 (s, 6H), 2.92-2.66 (m, 8H), 1.37 (d, 3H). | rac-(R)-5-(5- (dimethylcarbamoyl) pyridin-2-yl)-N-(4-(3,3- dimethylureido)benzyl)- 1-(1-methoxypropan-2- yl)-4,5,6,7-tetrahydro- 1H-pyrazolo[4,3- c]pyridine-3- carboxamide | 563.4 |
| 93 | Ex. 1^{a} | 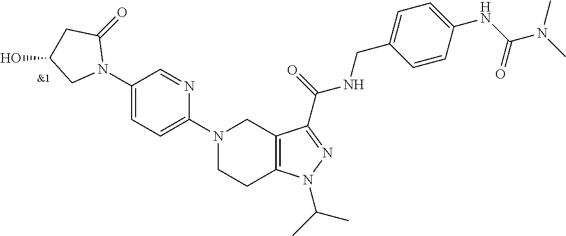 [SMILES: CC(C)n1nc(C(=O)NCc2ccc(NC(=O)N(C)C)cc2)c2c1CCN(c1ccc(N3C[C@H](O)CC3=O)cn1)C2] | rac-(R)-N-(4-(3,3- dimethylureido)benzyl)- 5-(5-(4-hydroxy-2- oxopyrrolidin-1- yl)pyridin-2-yl)-1- isopropyl-4,5,6,7- tetrahydro-1H- pyrazolo[4,3-c]pyridine- 3-carboxamide | 561.3 |
| 94 | Ex. 43 | 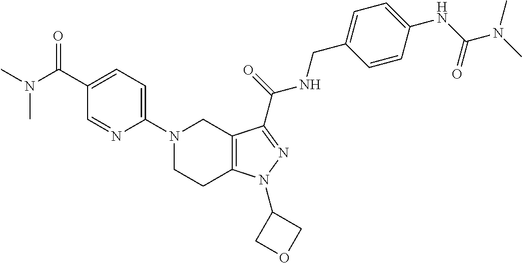 ¹H NMR (400 MHz, (CD₃)₂SO) δ 8.63 (t, 1H), 8.26-8.19 (m, 2H), 7.63 (dd, 1H), 7.42-7.36 (m, 2H), 7.22-7.16 (m, 2H), 6.89 (d, 1H), 5.61-5.46 (m, 1H), 4.97 (t, 2H), 4.87 (t, 2H), 4.76 (s, 2H), 4.41-4.30 (m, 2H), 3.92 (t, 2H), 2.96 (s, 6H), 2.90 (s, 6H), 2.80-2.72 (m, 2H). | 5-(5- (dimethylcarbamoyl) pyridin-2-yl)-N-(4-(3,3- dimethylureido)benzyl)- 1-(oxetan-3-yl)-4,5,6,7- tetrahydro-1H- pyrazolo[4,3-c]pyridine- 3-carboxamide | 547.3 |
| 95 | Ex. 43 | 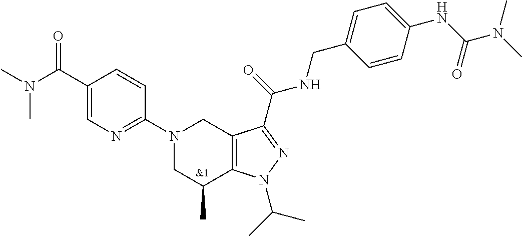 ¹H NMR (400 MHz, CDCl₃) δ 8.31 (d, 1H), 7.63 (dd, 1H), 7.38-7.33 (m, 2H), 7.32- 7.28 (m, 2H), 7.14 (t, 1H), 6.78 (d, 1H), 6.31 (s, 1H), 4.98 (d, 1H), 4.85-4.78 (m, 1H), 4.58-4.53 (m, 2H), 4.46-4.36 (m, 2H), 3.34 (dd, 1H), 3.09 (s, 7H), 3.03 (s, 6H), 1.48-1.44 (m, 6H), 1.30-1.26 (m, 3H). | rac-(R)-5-(5- (dimethylcarbamoyl) pyridin-2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 547.3 |
| 96 | Ex. 43 | 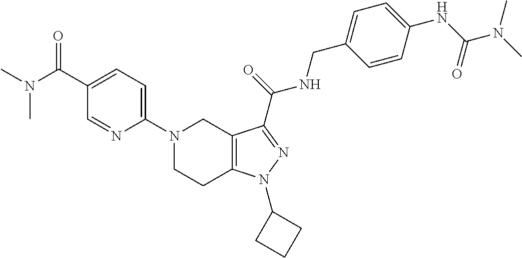 ¹H NMR (400 MHz, (CD₃)₂SO) δ 8.46 (t, 1H), 8.27-8.19 (m, 2H), 7.63 (dd, 1H), 7.41-7.36 (m, 2H), 7.20-7.15 (m, 2H), 6.88 (d, 1H), 4.85-4.69 (m, 3H), 4.35 (d, 2H), 3.92 (t, 2H), 2.96 (s, 6H), 2.90 (s, 6H), 2.77 (t, 2H), 2.61-2.52 (m, 2H), 2.37- 2.28 (m, 2H), 1.83-1.71 (m, 2H). | 1-cyclobutyl-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 545.3 |
| 97 | Ex. 1^{a} | 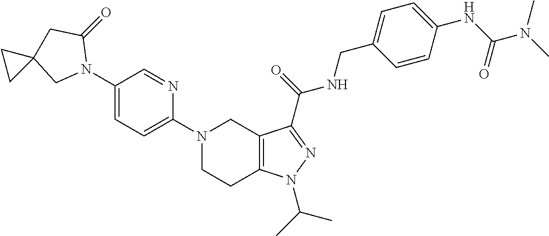 [SMILES: CC(C)n1nc(C(=O)NCc2ccc(NC(=O)N(C)C)cc2)c2c1CCN(c1ccc(N3CC4(CC4)CC3=O)cn1)C2] | N-(4-(3,3- dimethylureido)benzyl)- 1-isopropyl-5-(5-(6-oxo- 5-azaspiro[2.4]heptan-5- yl)pyridin-2-yl)-4,5,6,7- tetrahydro-1H- pyrazolo[4,3-c]pyridine- 3-carboxamide | 571.4 |
| 98 | Ex. 1^{a} | 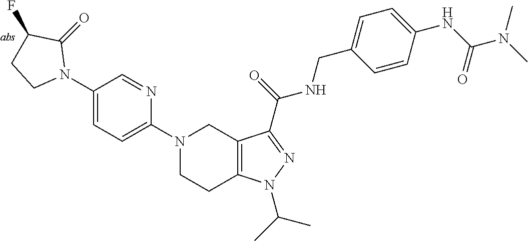 [SMILES: CC(C)n1nc(C(=O)NCc2ccc(NC(=O)N(C)C)cc2)c2c1CCN(c1ccc(N3CC[C@@H](F)C3=O)cn1)C2] | (R)-N-(4-(3,3- dimethylureido)benzyl)- 5-(5-(3-fluoro-2- oxopyrrolidin-1- yl)pyridin-2-yl)-1- isopropyl-4,5,6,7- tetrahydro-1H- pyrazolo[4,3-c]pyridine- 3-carboxamide | 563.4 |
| 99 | Ex. 1^{a} | 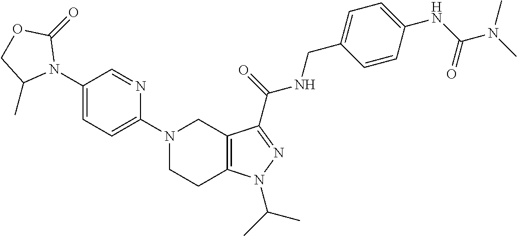 [SMILES: CC1COC(=O)N1c1ccc(N2CCc3c(c(C(=O)NCc4ccc(NC(=O)N(C)C)cc4)nn3C(C)C)C2)nc1] | N-(4-(3,3- dimethylureido)benzyl)- 1-isopropyl-5-(5-(4- methyl-2-oxooxazolidin- 3-yl)pyridin-2-yl)-4,5,6,7- tetrahydro-1H- pyrazolo[4,3-c]pyridine- 3-carboxamide | 561.4 |
| 100 | Ex. 1^{ac} | 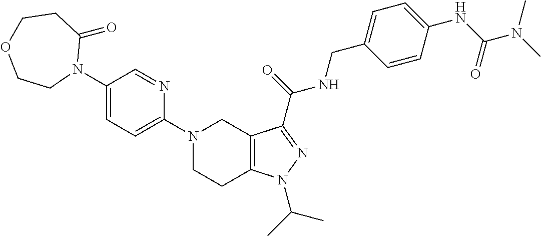 [SMILES: CC(C)n1nc(C(=O)NCc2ccc(NC(=O)N(C)C)cc2)c2c1CCN(c1ccc(N3CCOCCC3=O)cn1)C2] | N-(4-(3,3- dimethylureido)benzyl)- 1-isopropyl-5-(5-(5-oxo- 1,4-oxazepan-4- yl)pyridin-2-yl)-4,5,6,7- tetrahydro-1H- pyrazolo[4,3-c]pyridine- 3-carboxamide | 575.4 |
| 101 | Ex. 43 | 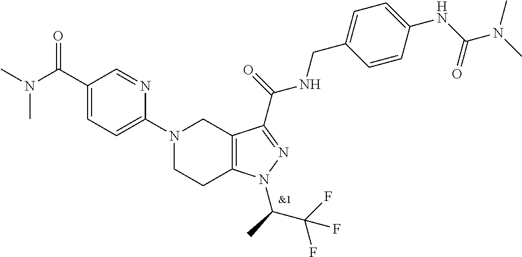 ¹H NMR (400 MHz, (CD₃)₂SO) δ 8.48 (t, 1H), 8.27-8.20 (m, 2H), 7.64 (dd, 1H), 7.42-7.36 (m, 2H), 7.20-7.14 (m, 2H), 6.91 (d, 1H), 5.45-5.31 (m, 1H), 4.92- 4.61 (m, 2H), 4.35 (d, 2H), 4.14-4.04 (m, 1H), 3.88-3.77 (m, 1H), 2.99-2.93 (m, 7H), 2.91 (s, 6H), 2.77-2.64 (m, 1H), 1.71 (d, 3H). | rac-(R)-5-(5- (dimethylcarbamoyl) pyridin-2-yl)-N-(4-(3,3- dimethylureido)benzyl)- 1-(1,1,1-trifluoropropan- 2-yl)-4,5,6,7-tetrahydro- 1H-pyrazolo[4,3- c]pyridine-3- carboxamide | 587.4 |
| 102 | Ex. 102 | 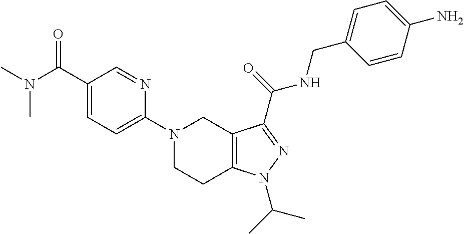 [SMILES: CC(C)n1nc(C(=O)NCc2ccc(N)cc2)c2c1CCN(c1ccc(C(=O)N(C)C)cn1)C2] ¹H NMR (400 MHz, (CD₃)₂SO) δ 8.24 (d, 1H), 8.13 (t, 1H), 7.64 (dd, 1H), 7.03-6.96 (m, 2H), 6.88 (d, 1H), 6.52-6.46 (m, 2H), 4.95 (s, 2H), 4.78-4.69 (m, 2H), 4.54- 4.41 (m, 1H), 4.24 (d, 2H), 3.94 (t, 2H), 2.97 (s, 6H), 2.81 (t, 2H), 1.38 (d, 6H). | N-(4-aminobenzyl)-5-(5- (dimethylcarbamoyl) pyridin-2-yl)-1-isopropyl- 4,5,6,7-tetrahydro-1H- pyrazolo[4,3-c]pyridine- 3-carboxamide | 462.3 |
| 103 | Ex. 1^{ac} | 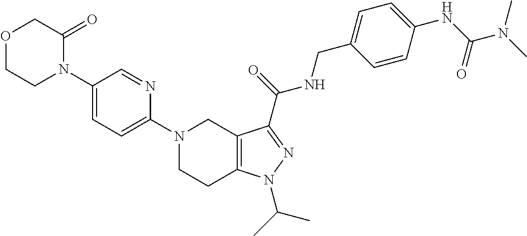 [SMILES: CC(C)n1nc(C(=O)NCc2ccc(NC(=O)N(C)C)cc2)c2c1CCN(c1ccc(N3CCOCC3=O)cn1)C2] | N-(4-(3,3- dimethylureido)benzyl)- 1-isopropyl-5-(5-(3- oxomorpholino)pyridin- 2-yl)-4,5,6,7-tetrahydro- 1H-pyrazolo[4,3- c]pyridine-3- carboxamide | 561.3 |
| 104 | Ex. 9 | 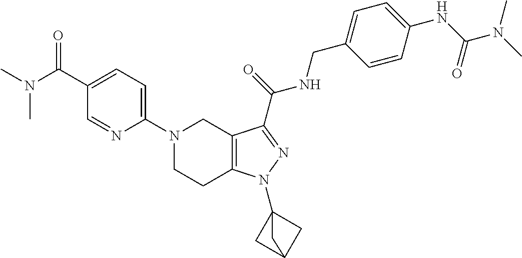 ¹H NMR (400 MHz, CDCl₃) δ 8.30 (d, 1H), 7.61 (dd, 1H), 7.38-7.31 (m, 2H), 7.29- 7.24 (m, 2H), 6.75 (d, 1H), 4.75 (s, 2H), 4.53 (s, 2H), 4.13-4.08 (m, 2H), 3.07 (s, 6H), 3.02 (s, 6H), 2.90-2.85 (m, 2H), 2.59 (s, 1H), 2.31 (s, 6H). | 1-(bicyclo[1.1.1]pentan- 1-yl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 557.4 |
| 105 | Ex. 43 | 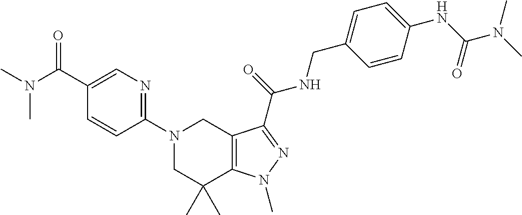 ¹H NMR (400 MHz, CDCl₃) δ 8.31 (d, 1H), 7.62 (dd, 1H), 7.37-7.32 (m, 2H), 7.29- 7.26 (m, 2H), 7.08 (t, 1H), 6.77 (d, 1H), 6.33 (s, 1H), 4.70 (s, 2H), 4.54 (d, 2H), 3.90 (s, 3H), 3.84 (s, 2H), 3.08 (s, 6H), 3.03 (s, 6H), 1.37 (s, 6H). | 5-(5- (dimethylcarbamoyl) pyridin-2-yl)-N-(4-(3,3- dimethylureido)benzyl)- 1,7,7-trimethyl-4,5,6,7- tetrahydro-1H- pyrazolo[4,3-c]pyridine- 3-carboxamide | 533.3 |
| 106 | Ex. 5 | 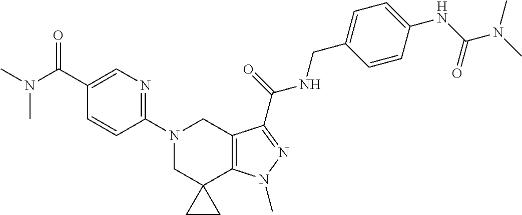 ¹H NMR (400 MHz, (CD₃)₂SO) δ 8.46 (t, 1H), 8.24-8.19 (m, 2H), 7.61 (dd, 1H), 7.41-7.34 (m, 2H), 7.19-7.12 (m, 2H), 6.88 (d, 1H), 4.81 (s, 2H), 4.32 (d, 2H), 3.74 (s, 2H), 3.67 (s, 3H), 2.97 (s, 6H), 2.90 (s, 6H), 1.42 (t, 2H), 1.02 (t, 2H). | 5′-(5- (dimethylcarbamoyl) pyridin-2-yl)-N-(4-(3,3- dimethylureido)benzyl)- 1′-methyl-1′,4′,5′,6′- tetrahydrospiro [cyclopropane- 1,7′-pyrazolo[4,3- c]pyridine]-3′- carboxamide | 531.4 |
| 107 | Ex. 1^{a} | 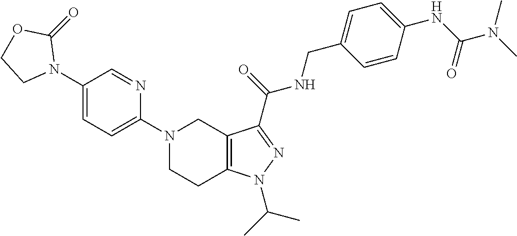 [SMILES: CC(C)n1nc(C(=O)NCc2ccc(NC(=O)N(C)C)cc2)c2c1CCN(c1ccc(N3CCOC3=O)cn1)C2] | N-(4-(3,3- dimethylureido)benzyl)- 1-isopropyl-5-(5-(2- oxooxazolidin-3- yl)pyridin-2-yl)-4,5,6,7- tetrahydro-1H- pyrazolo[4,3-c]pyridine- 3-carboxamide | 547.4 |
| 108 | Ex. 145 | 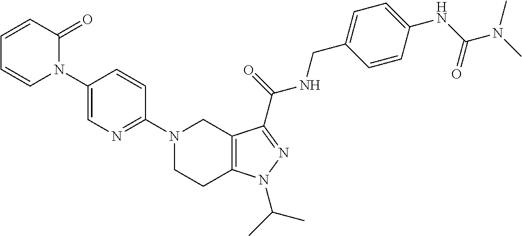 [SMILES: CC(C)n1nc(C(=O)NCc2ccc(NC(=O)N(C)C)cc2)c2c1CCN(c1ccc(-n3ccccc3=O)cn1)C2] ¹H NMR (400 MHz, (CD₃)₂SO) δ 8.35 (t, 1H), 8.24 (s, 1H), 8.11 (d, 1H), 7.66-7.60 (m, 2H), 7.53-7.46 (m, 1H), 7.41-7.36 (m, 2H), 7.20-7.14 (m, 2H), 7.01 (d, 1H), 6.48-6.44 (m, 1H), 6.33-6.27 (m, 1H), 4.75 (s, 2H), 4.55-4.44 (m, 1H), 4.34 (d, 2H), 3.95 (t, 2H), 2.90 (s, 6H), 2.87-2.81 (m, 2H), 1.39 (d, 6H). | N-(4-(3,3- dimethylureido)benzyl)- 1-isopropyl-5-(2-oxo-2H- [1,3′-bipyridin]-6′-yl)- 4,5,6,7-tetrahydro-1H- pyrazolo[4,3-c]pyridine- 3-carboxamide | 555.5 |
| 109 | Ex. 1^{ac} | 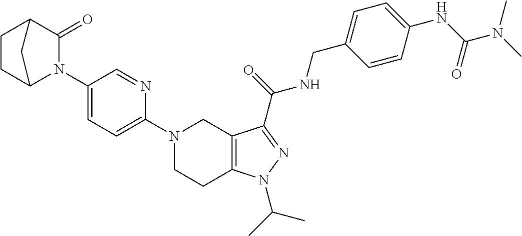 [SMILES: CC(C)n1nc(C(=O)NCc2ccc(NC(=O)N(C)C)cc2)c2c1CCN(c1ccc(N3C(=O)C4CCC3C4)cn1)C2] | N-(4-(3,3- dimethylureido)benzyl)- 1-isopropyl-5-(5-(3-oxo- 2- azabicyclo[2.2.1]heptan- 2-yl)pyridin-2-yl)-4,5,6,7- tetrahydro-1H- pyrazolo[4,3-c]pyridine- 3-carboxamide | 571.4 |
| 110 | Ex. 1^{a} | 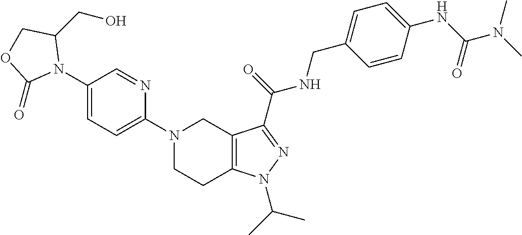 [SMILES: CC(C)n1nc(C(=O)NCc2ccc(NC(=O)N(C)C)cc2)c2c1CCN(c1ccc(N3C(=O)OCC3CO)cn1)C2] | N-(4-(3,3- dimethylureido)benzyl)- 5-(5-(4-(hydroxymethyl)- 2-oxooxazolidin-3- yl)pyridin-2-yl)-1- isopropyl-4,5,6,7- tetrahydro-1H- pyrazolo[4,3-c]pyridine- 3-carboxamide | 577.4 |
| 111^{∧} | Ex. 113 | 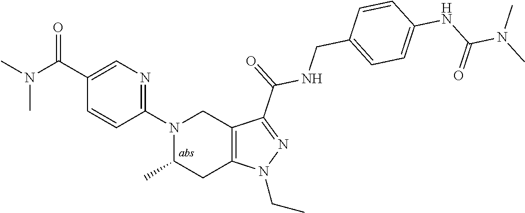 ¹H NMR (400 MHz, (CD₃)₂SO) δ 8.46 (t, 1H), 8.26 (d, 1H), 8.22 (s, 1H), 7.65 (dd, 1H), 7.40-7.35 (m, 2H), 7.20-7.14 (m, 2H), 6.84 (d, 1H), 5.31-5.22 (m, 1H), 5.14 (d, 1H), 4.32 (d, 2H), 4.17-4.02 (m, 3H), 3.01-2.92 (m, 7H), 2.90 (s, 6H), 2.73 (d, 1H), 1.32 (t, 3H), 1.06 (d, 3H). | (S)-5-(5- (dimethylcarbamoyl) pyridin-2-yl)-N-(4-(3,3- dimethylureido)benzyl)- 1-ethyl-6-methyl-4,5,6,7- tetrahydro-1H- pyrazolo[4,3-c]pyridine- 3-carboxamide | 533.3 |
| 112^{∧} | Ex. 113 | 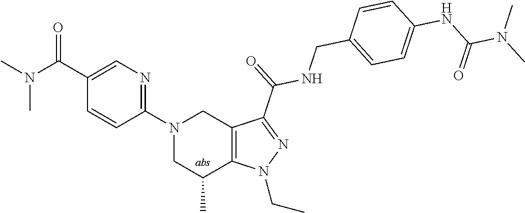 ¹H NMR (400 MHz, CDCl₃) δ 8.31 (d, 1H), 7.63 (dd, 1H), 7.38-7.33 (m, 2H), 7.32- 7.28 (m, 2H), 7.17-7.12 (m, 1H), 6.81- 6.76 (m, 1H), 6.34-6.28 (m, 1H), 5.01- 4.93 (m, 1H), 4.85-4.76 (m, 1H), 4.57- 4.53 (m, 2H), 4.46-4.34 (m, 2H), 3.37- 3.30 (m, 1H), 3.09 (s, 7H), 3.03 (s, 6H), 1.51-1.41 (m, 6H), 1.30-1.25 (m, 3H). | (R)-5-(5- (dimethylcarbamoyl) pyridin-2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 547.5 |
| 113 | Ex. 113 | 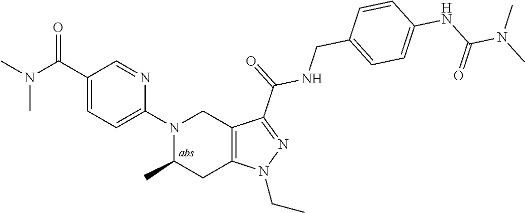 ¹H NMR (400 MHz, (CD₃)₂SO) δ 8.47 (t, 1H), 8.26 (d, 1H), 8.23 (s, 1H), 7.65 (dd, 1H), 7.41-7.34 (m, 2H), 7.21-7.15 (m, 2H), 6.85 (d, 1H), 5.31-5.22 (m, 1H), 5.14 (d, 1H), 4.33 (d, 2H), 4.17-4.01 (m, 3H), 3.01-2.93 (m, 7H), 2.90 (s, 6H), 2.77- 2.69 (m, 1H), 1.32 (t, 3H), 1.06 (d, 3H). | (R)-5-(5- (dimethylcarbamoyl) pyridin-2-yl)-N-(4-(3,3- dimethylureido)benzyl)- 1-ethyl-6-methyl-4,5,6,7- tetrahydro-1H- pyrazolo[4,3-c]pyridine- 3-carboxamide | 533.3 |
| 114 | Ex. 113^{d} | 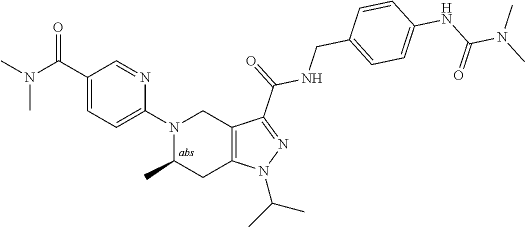 ¹H NMR* (600 MHz, (CD₃)₂SO) δ 8.36 (t, 1H), 8.27-8.25 (m, 1H), 8.23 (s, 1H), 7.67 (dd, 1H), 7.41-7.37 (m, 2H), 7.21-7.16 (m, 2H), 6.87 (d, 1H), 5.29-5.21 (m, 1H), 5.13 (d, 1H), 4.56-4.47 (m, 1H), 4.38- 4.30 (m, 2H), 4.18-4.09 (m, 1H), 3.00- 2.95 (m, 7H), 2.92-2.89 (m, 6H), 2.77 (d, 1H), 1.41 (dd, 6H), 1.07 (d, 3H). | (R)-5-(5- (dimethylcarbamoyl) pyridin-2-yl)-N-(4-(3,3- dimethylureido)benzyl)- 1-isopropyl-6-methyl- 4,5,6,7-tetrahydro-1H- pyrazolo[4,3-c]pyridine- 3-carboxamide | 547.5 |
| 115 | Ex. 113^{d} | 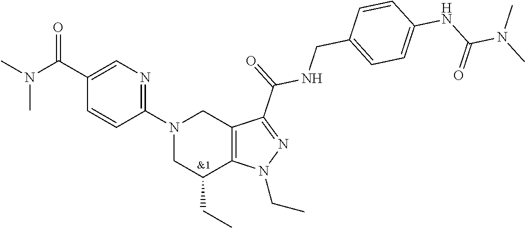 ¹H NMR* (600 MHz, (CD₃)₂SO) δ 8.46 (t, 1H), 8.26-8.20 (m, 2H), 7.66 (dd, 1H), 7.41-7.36 (m, 2H), 7.19-7.15 (m, 2H), 6.85 (d, 1H), 5.00 (d, 1H), 4.76 (d, 1H), 4.39- 4.28 (m, 2H), 4.24 (d, 1H), 4.15-4.05 (m, 2H), 3.21-3.16 (m, 1H), 3.04-2.94 (m, 7H), 2.90 (s, 6H), 1.62-1.53 (m, 1H), 1.48- 1.37 (m, 4H), 1.03 (t, 3H). | rac-(R)-5-(5- (dimethylcarbamoyl) pyridin-2-yl)-N-(4-(3,3- dimethylureido)benzyl)- 1,7-diethyl-4,5,6,7- tetrahydro-1H- pyrazolo[4,3-c]pyridine- 3-carboxamide | 547.4 |
| 116 | Ex. 128^{de} | 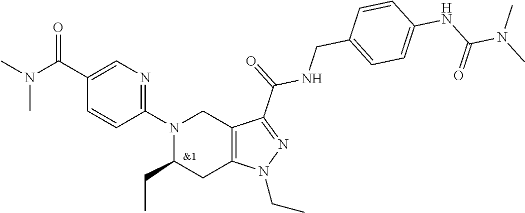 ¹H NMR* (600 MHz, (CD₃)₂SO) δ 8.45 (t, 1H), 8.25-8.20 (m, 2H), 7.64 (dd, 1H), 7.41-7.35 (m, 2H), 7.20-7.14 (m, 2H), 6.88 (d, 1H), 5.23 (d, 1H), 5.02 (s, 1H), 4.38- 4.28 (m, 2H), 4.17-4.03 (m, 3H), 3.02- 2.94 (m, 6H), 2.93-2.86 (m, 7H), 2.80 (d, 1H), 1.55-1.40 (m, 2H), 1.32 (t, 3H), 0.80 (t, 3H). | rac-(R)-5-(5- (dimethylcarbamoyl) pyridin-2-yl)-N-(4-(3,3- dimethylureido)benzyl)- 1,6-diethyl-4,5,6,7- tetrahydro-1H- pyrazolo[4,3-c]pyridine- 3-carboxamide | 547.5 |
| 117^{∧} | Ex. 128^{de} | 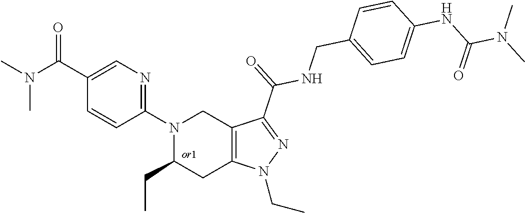 ¹H NMR (600 MHz, (CD₃)₂SO) δ 8.45 (t, 1H), 8.25-8.21 (m, 2H), 7.63 (dd, 1H), 7.40-7.36 (m, 2H), 7.19-7.15 (m, 2H), 6.86 (d, 1H), 5.23 (d, 1H), 5.02 (s, 1H), 4.37- 4.28 (m, 2H), 4.16-4.02 (m, 3H), 2.97 (s, 6H), 2.93-2.87 (m, 7H), 2.83-2.77 (m, 1H), 1.55-1.38 (m, 2H), 1.31 (t, 3H), 0.80 (t, 3H). | rel-(R or S)-5-(5- (dimethylcarbamoyl) pyridin-2-yl)-N-(4-(3,3- dimethylureido)benzyl)- 1,6-diethyl-4,5,6,7- tetrahydro-1H- pyrazolo[4,3-c]pyridine- 3-carboxamide | 547.3 |
| 118^{∧} | Ex. 128^{de} | 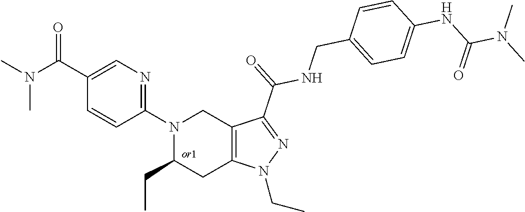 ¹H NMR (600 MHz, (CD₃)₂SO) δ 8.45 (t, 1H), 8.25-8.21 (m, 2H), 7.63 (dd, 1H), 7.40- 7.36 (m, 2H), 7.19-7.15 (m, 2H), 6.86 (d, 1H), 5.23 (d, 1H), 5.03 (s, 1H), 4.38-4.27 (m, 2H), 4.16-4.02 (m, 3H), 2.97 (s, 6H), 2.93-2.86 (m, 7H), 2.82-2.77 (m, 1H), 1.56-1.39 (m, 2H), 1.31 (t, 3H), 0.80 (t, 3H). | rel-(R or S)-5-(5- (dimethylcarbamoyl) pyridin-2-yl)-N-(4-(3,3- dimethylureido)benzyl)- 1,6-diethyl-4,5,6,7- tetrahydro-1H- pyrazolo[4,3-c]pyridine- 3-carboxamide | 547.3 |
| 119^{∧} | Ex. 113 | 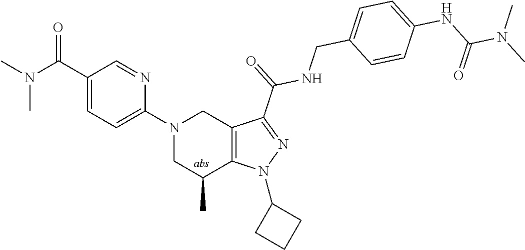 ¹H NMR (400 MHz, (CD₃)₂SO) δ 8.50-8.43 (m, 1H), 8.21 (s, 2H), 7.61 (d, 1H), 7.40- 7.33 (m, 2H), 7.16 (d, 2H), 6.86 (d, 1H), 5.04 (d, 1H), 4.90-4.77 (m, 1H), 4.41- 4.18 (m, 4H), 3.23-3.11 (m, 2H), 2.95 (s, 6H), 2.88 (s, 6H), 2.78-2.62 (m, 2H), 2.39- 2.26 (m, 2H), 1.85-1.69 (m, 2H), 1.14 (d, 3H). | (S)-1-cyclobutyl-5-(5- (dimethylcarbamoyl) pyridin-2-yl)-N-(4-(3,3- dimethylureido)benzyl)- 7-methyl-4,5,6,7- tetrahydro-1H- pyrazolo[4,3-c]pyridine- 3-carboxamide | 559.3 |
| 120 | Ex. 77 | 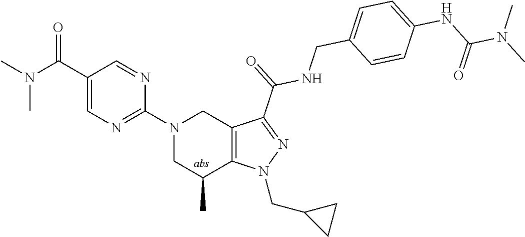 ¹H NMR (400 MHz, (CD₃)₂SO) δ 8.55- 8.48 (m, 2H), 8.42 (t, 1H), 8.23 (s, 1H), 7.41- 7.36 (m, 2H), 7.20-7.14 (m, 2H), 5.44 (d, 1H), 4.76-4.67 (m, 1H), 4.42-4.27 (m, 3H), 4.11-4.01 (m, 1H), 3.93-3.80 (m, 1H), 3.49-3.41 (m, 1H), 3.29-3.20 (m, 1H), 3.00 (s, 6H), 2.92-2.87 (m, 6H), 1.35- 1.22 (m, 1H), 1.16 (d, 3H), 0.58-0.46 (m, 2H), 0.45-0.37 (m, 2H). | (S)-1- (cyclopropylmethyl)-5- (5- (dimethylcarbamoyl) pyrimidin-2-yl)-N-(4-(3,3- dimethylureido)benzyl)- 7-methyl-4,5,6,7- tetrahydro-1H- pyrazolo[4,3-c]pyridine- 3-carboxamide | 560.3 |
| 121 | P44; Ex. 30^{f} | 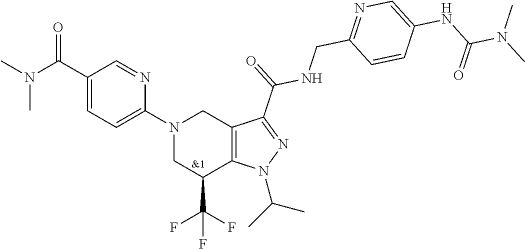 1H NMR (600 MHz, (CD₃)₂SO) δ 8.65- 8.61 (m, 2H), 8.53 (s, 1H), 8.28-8.26 (m, 1H), 7.92-7.85 (m, 1H), 7.68 (dd, 1H), 7.29 (d, 1H), 6.83 (d, 1H), 5.28 (d, 1H), 5.06 (d, 1H), 4.74-4.65 (m, 1H), 4.58-4.45 (m, 3H), 4.30-4.23 (m, 1H), 3.30-3.28 (m, 1H), 2.98 (s, 6H), 2.93 (s, 6H), 1.45 (dd, 6H). | rac-(R)-5-(5- (dimethylcarbamoyl) pyridin-2-yl)-N-((5-(3,3- dimethylureido)pyridin- 2-yl)methyl)-1-isopropyl- 7-(trifluoromethyl)- 4,5,6,7-tetrahydro-1H- pyrazolo[4,3-c]pyridine- 3-carboxamide | 602.6 |
| 122^{∧} | Ex. 42 | 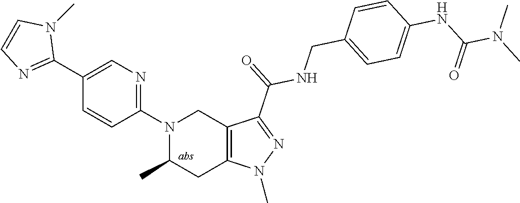 [SMILES: C[C@@H]1Cc2c(c(C(=O)NCc3ccc(NC(=O)N(C)C)cc3)nn2C)CN1c1ccc(-c2nccn2C)cn1] ¹H NMR (400 MHz, (CD₃)₂SO) δ 8.50 (t, 1H), 8.46 (d, 1H), 8.22 (s, 1H), 7.87 (dd, 1H), 7.42-7.35 (m, 2H), 7.21-7.14 (m, 3H), 6.96-6.91 (m, 2H), 5.32-5.22 (m, 1H), 5.21-5.13 (m, 1H), 4.41-4.26 (m, 2H), 4.10 (d, 1H), 3.78 (s, 3H), 3.72 (s, 3H), 3.01-2.88 (m, 7H), 2.77-2.65 (m, 1H), 1.09 (d, 3H). | (R)-N-(4-(3,3- dimethylureido)benzyl)- 1,6-dimethyl-5-(5-(1- methyl-1H-imidazol-2- yl)pyridin-2-yl)-4,5,6,7- tetrahydro-1H- pyrazolo[4,3-c]pyridine- 3-carboxamide | 528.3 |
| 123^{∧} | Ex. 30^{f} | 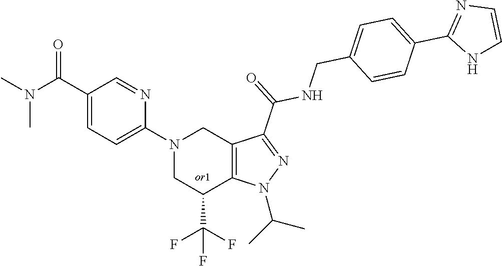 [SMILES: CC(C)n1nc(C(=O)NCc2ccc(-c3ncc[nH]3)cc2)c2c1[C@H](C(F)(F)F)CN(c1ccc(C(=O)N(C)C)cn1)C2] ¹H NMR (600 MHz, (CD₃)₂SO) δ 12.45 (s, 1H), 8.72-8.67 (m, 1H), 8.31-8.26 (m, 1H), 7.90-7.85 (m, 2H), 7.72-7.67 (m, 1H), 7.41-7.36 (m, 2H), 7.27-6.96 (m, 2H), 6.88-6.81 (m, 1H), 5.28 (d, 1H), 5.07 (d, 1H), 4.73-4.64 (m, 1H), 4.59-4.40 (m, 3H), 4.33-4.24 (m, 1H), 3.29-3.27 (m, 1H), 2.98 (s, 6H), 1.50-1.40 (m, 6H). | rel-(R or S)-N-(4-(1H- imidazol-2-yl)benzyl)-5- (5- (dimethylcarbamoyl) pyridin-2-yl)-1-isopropyl-7- (trifluoromethyl)-4,5,6,7- tetrahydro-1H- pyrazolo[4,3-c]pyridine- 3-carboxamide | 581.3 |
| 124^{∧} | Ex. 30^{f} | 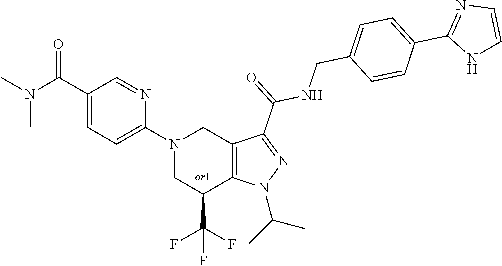 [SMILES: CC(C)n1nc(C(=O)NCc2ccc(-c3ncc[nH]3)cc2)c2c1[C@@H](C(F)(F)F)CN(c1ccc(C(=O)N(C)C)cn1)C2] ¹H NMR (600 MHz, (CD₃)₂SO) δ 12.45 (s, 1H), 8.69 (t, 1H), 8.27 (d, 1H), 7.90-7.85 (m, 2H), 7.69 (dd, 1H), 7.41-7.37 (m, 2H), 7.27-6.96 (m, 2H), 6.84 (d, 1H), 5.28 (d, 1H), 5.07 (d, 1H), 4.72-4.64 (m, 1H), 4.58- 4.40 (m, 3H), 4.29 (d, 1H), 3.31-3.28 (m, 1H), 2.98 (s, 6H), 1.50-1.38 (m, 6H). | rel-(R or S)-N-(4-(1H- imidazol-2-yl)benzyl)-5- (5- (dimethylcarbamoyl) pyridin-2-yl)-1-isopropyl-7- (trifluoromethyl)-4,5,6,7- tetrahydro-1H- pyrazolo[4,3-c]pyridine- 3-carboxamide | 581.3 |
| 125 | Ex. 125 | 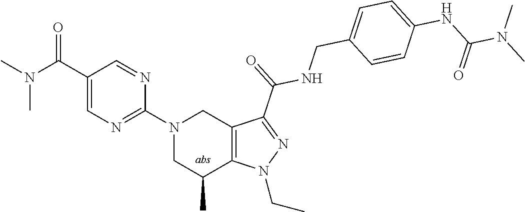 ¹H NMR (400 MHz, (CD₃)₂SO) δ 8.52 (s, 2H), 8.45 (t, 1H), 8.23 (s, 1H), 7.41-7.35 (m, 2H), 7.19-7.13 (m, 2H), 5.41 (d, 1H), 4.67 (dd, 1H), 4.42-4.26 (m, 3H), 4.16- 4.04 (m, 2H), 3.48 (dd, 1H), 3.28-3.19 (m, 1H), 2.99 (br s, 6H), 2.90 (s, 6H), 1.38 (t, 3H), 1.16 (d, 3H). | (S)-5-(5- (dimethylcarbamoyl) pyrimidin-2-yl)-N-(4-(3,3- dimethylureido)benzyl)- 1-ethyl-7-methyl-4,5,6,7- tetrahydro-1H- pyrazolo[4,3-c]pyridine- 3-carboxamide | 534.3 |
| 126 | Ex. 126 | 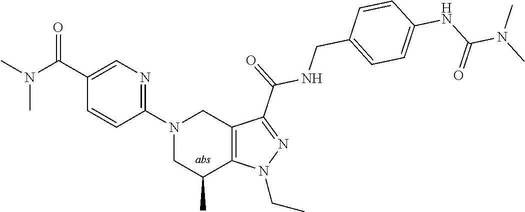 ¹H NMR (400 MHz, (CD₃)₂SO) δ 8.45 (t, 1H), 8.25-8.21 (m, 2H), 7.64 (dd, 1H), 7.41-7.35 (m, 2H), 7.19-7.13 (m, 2H), 6.89 (d, 1H), 5.05 (d, 1H), 4.41-4.25 (m, 4H), 4.17-4.04 (m, 2H), 3.43-3.36 (m, 1H), 3.26-3.17 (m, 1H), 2.98 (br s, 6H), 2.90 (s, 6H), 1.38 (t, 3H), 1.20 (d, 3H). | (S)-5-(5- (dimethylcarbamoyl) pyridin-2-yl)-N-(4-(3,3- dimethylureido)benzyl)- 1-ethyl-7-methyl-4,5,6,7- tetrahydro-1H- pyrazolo[4,3-c]pyridine- 3-carboxamide | 533.4 |
| 127^{∧} | Ex. 127 | 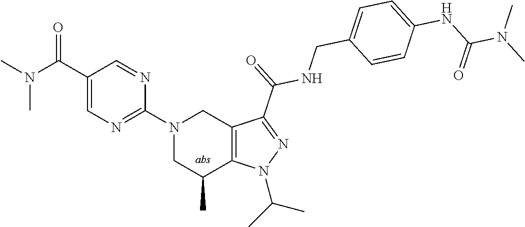 ¹H NMR (400 MHz, (CD₃)₂SO) δ 8.52 (s, 2H), 8.34 (t, 1H), 8.23 (s, 1H), 7.41-7.36 (m, 2H), 7.19-7.14 (m, 2H), 5.47 (d, 1H), 4.75 (dd, 1H), 4.58-4.46 (m, 1H), 4.40- 4.29 (m, 3H), 3.42 (dd, 1H), 3.29-3.23 (m, 1H) 3.04-2.94 (m, 6H), 2.91 (s, 6H), 1.38 (dd, 6H), 1.11 (d, 3H). | (S)-5-(5- dimethylcarbamoyl) pyrimidin-2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 548.9 |
| 128^{∧} | Ex. 128 | 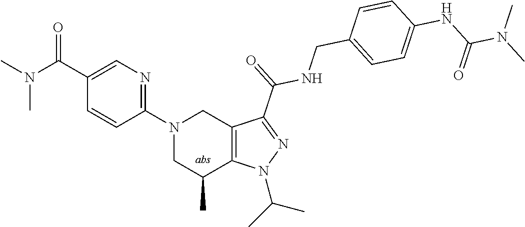 ¹H NMR (400 MHz, (CD₃)₂SO) δ 8.29 (t, 1H), 8.18-8.16 (m, 2H), 7.57 (dd, 1H), 7.34-7.29 (m, 2H), 7.14-7.08 (m, 2H), 6.82 (d, 1H), 5.03 (d, 1H), 4.50-4.41 (m, 1H), 4.39-4.23 (m, 3H), 4.17 (d, 1H), 3.36- 3.35 (m, 1H), 3.18-3.12 (m, 1H), 2.91 (s, 6H), 2.84 (s, 6H), 1.35 (dd, 6H), 1.13 (d, 3H) | (S)-5-(5- (dimethylcarbamoyl) pyridin-2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 547.3 |
| 129 | Ex. 77 | 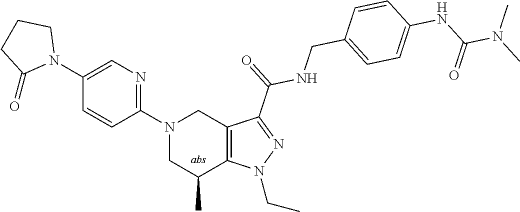 [SMILES: CCn1nc(C(=O)NCc2ccc(NC(=O)N(C)C)cc2)c2c1[C@@H](C)CN(c1ccc(N3CCCC3=O)cn1)C2] ¹H NMR (400 MHz, (CD₃)₂SO) δ 8.40 (t, 1H), 8.25 (d, 1H), 8.19 (s, 1H), 7.84 (dd, 1H), 7.37-7.31 (m, 2H), 7.16-7.10 (m, 2H), 6.86 (d, 1H), 4.86 (d, 1H), 4.32-4.26 (m, 2H), 4.24-4.14 (m, 2H), 4.10-4.00 (m, 2H), 3.77-3.70 (m, 2H), 3.18-3.11 (m, 1H), 2.87 (s, 6H), 2.42-2.37 (m, 3H), 2.07-1.96 (m, 2H), 1.35 (t, 3H), 1.18 (d, 3H). | (S)-N-(4-(3,3- dimethylureido)benzyl)- 1-ethyl-7-methyl-5-(5-(2- oxopyrrolidin-1- yl)pyridin-2-yl)-4,5,6,7- tetrahydro-1H- pyrazolo[4,3-c]pyridine- 3-carboxamide | 545.3 |
| 130 | Ex. 23^{g} | 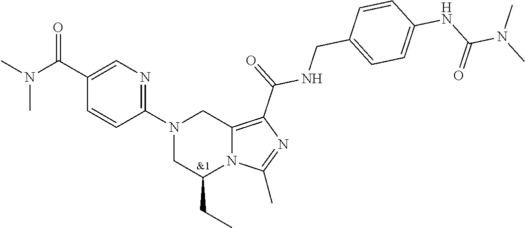 ¹H NMR (400 MHz, CD₃OD) δ 8.34 (d, 1H), 7.76 (dd, 1H), 7.38-7.33 (m, 2H), 7.30- 7.25 (m, 2H), 6.91 (d, 1H), 5.32-5.12 (m, 2H), 4.71-4.63 (m, 1H), 4.61-4.55 (m, 1H), 4.51 (s, 2H), 4.37-4.28 (m, 1H), 3.15- 3.08 (m, 6H), 3.02 (s, 6H), 2.42 (s, 3H), 1.80-1.69 (m, 2H), 1.09 (t, 3H). | rac-(R)-7-(5- (dimethylcarbamoyl) pyridin-2-yl)-N-(4-(3,3- dimethylureido)benzyl)- 5-ethyl-3-methyl-5,6,7,8- tetrahydroimidazo[1,5- a]pyrazine-1- carboxamide | 533.3 |
| 131^{∧} | Ex. 23^{g} | 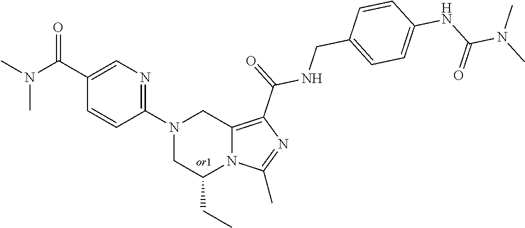 ¹H NMR (400 MHz, CD₃OD) δ 8.35-8.30 (m, 1H), 7.77-7.70 (m, 1H), 7.36-7.30 (m, 2H), 7.28-7.22 (m, 2H), 6.88 (d, 1H), 5.29-5.10 (m, 2H), 4.72-4.55 (m, 2H), 4.54-4.45 (m, 2H), 4.35-4.26 (m, 1H), 3.09 (s, 6H), 3.00 (s, 6H), 2.40 (s, 3H), 1.79- 1.66 (m, 2H), 1.06 (t, 3H). | rel-(R or S)-7-(5- (dimethylcarbamoyl) pyridin-2-yl)-N-(4-(3,3- dimethylureido)benzyl)- 5-ethyl-3-methyl-5,6,7,8- tetrahydroimidazo[1,5- a]pyrazine-1- carboxamide | 533.3 |
| 132 | Ex. 132 | 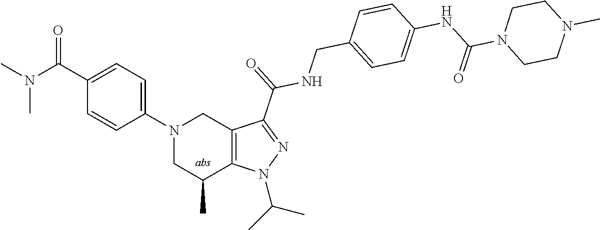 ¹H NMR (400 MHz, (CD₃)₂SO) δ 8.46 (s, 1H), 8.36 (t, 1H), 7.40-7.28 (m, 4H), 7.21- 7.12 (m, 2H), 7.00-6.91 (m, 2H), 4.59- 4.48 (m, 2H), 4.36-4.30 (m, 2H), 4.10 (d, 1H), 3.85-3.77 (m, 1H), 3.41 (t, 4H), 3.28- 3.16 (m, 2H), 2.97-2.91 (m, 6H), 2.29 (t, 4H), 2.18 (s, 3H), 1.47-1.37 (m, 6H), 1.28 (d, 3H). | (S)-5-(4- (dimethylcarbamoyl) phenyl)-1-isopropyl-7- methyl-N-(4-(4- methylpiperazine-1- carboxamido)benzyl)- 4,5,6,7-tetrahydro-1H- pyrazolo[4,3-c]pyridine- 3-carboxamide | 601.3 |
| 133 | Ex. 113 | 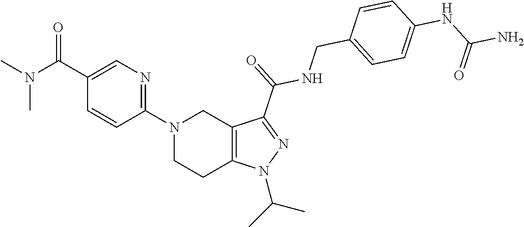 [SMILES: CC(C)n1nc(C(=O)NCc2ccc(NC(N)=O)cc2)c2c1CCN(c1ccc(C(=O)N(C)C)cn1)C2] ¹H NMR (400 MHz, (CD₃)₂SO) δ 8.42 (s, 1H), 8.27 (t, 1H), 8.18 (d, 1H), 7.58 (dd, 1H), 7.29-7.24 (m, 2H), 7.15-7.09 (m, 2H), 6.83 (d, 1H), 5.73 (s, 2H), 4.69 (s, 2H), 4.49-4.38 (m, 1H), 4.28 (d, 2H), 3.89 (t, 2H), 2.91 (s, 6H), 2.76 (t, 2H), 1.34 (d, 6H). | 5-(5- (dimethylcarbamoyl) pyridin-2-yl)-1-isopropyl-N- (4-ureidobenzyl)-4,5,6,7- tetrahydro-1H- pyrazolo[4,3-c]pyridine- 3-carboxamide | 505.4 |
| 134 | Ex. 113 | 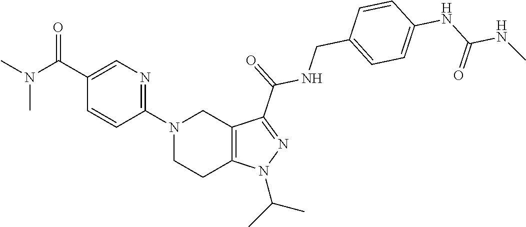 ¹H NMR (400 MHz, (CD₃)₂SO) δ 8.40 (s, 1H), 8.27 (t, 1H), 8.19 (d, 1H), 7.59 (dd, 1H), 7.30-7.24 (m, 2H), 7.15-7.10 (m, 2H), 6.84 (d, 1H), 5.91 (q, 1H), 4.70 (s, 2H), 4.50-4.36 (m, 1H), 4.28 (d, 2H), 3.90 (t, 2H), 2.92 (s, 6H), 2.77 (t, 2H), 2.57 (d, 3H), 1.34 (d, 6H). | 5-(5- (dimethylcarbamoyl) pyridin-2-yl)-1-isopropyl-N- (4-(3- methylureido)benzyl)- 4,5,6,7-tetrahydro-1H- pyrazolo[4,3-c]pyridine- 3-carboxamide | 519.3 |
| 135 | Ex. 43 | 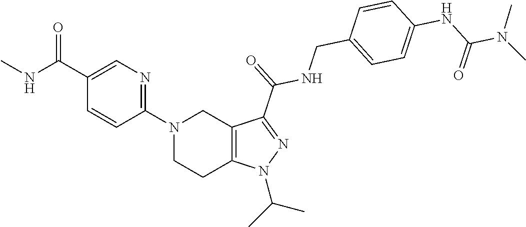 ¹H NMR (400 MHz, (CD₃)₂SO) δ 8.52 (d, 1H), 8.29 (t, 1H), 8.21-8.13 (m, 2H), 7.88 (dd, 1H), 7.35-7.30 (m, 2H), 7.14-7.07 (m, 2H), 6.83 (d, 1H), 4.69 (s, 2H), 4.48- 4.36 (m, 1H), 4.28 (d, 2H), 3.90 (t, 2H), 2.84 (s, 6H), 2.75 (t, 2H), 2.68 (d, 3H), 1.32 (d, 6H). | N-(4-(3,3- dimethylureido)benzyl)- 1-isopropyl-5-(5- (methylcarbamoyl) pyridin-2-yl)-4,5,6,7- tetrahydro-1H- pyrazolo[4,3-c]pyridine- 3-carboxamide | 519.5 |
| 136 | Ex. 136 | 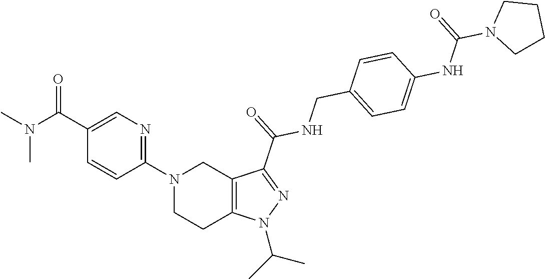 [SMILES: CC(C)n1nc(C(=O)NCc2ccc(NC(=O)N3CCCC3)cc2)c2c1CCN(c1ccc(C(=O)N(C)C)cn1)C2] | 5-(5- (dimethylcarbamoyl) pyridin-2-yl)-1-isopropyl-N- (4-(pyrrolidine-1- carboxamido)benzyl)- 4,5,6,7-tetrahydro-1H- pyrazolo[4,3-c]pyridine- 3-carboxamide | 559.4 |
| 137 | Ex. 193 | 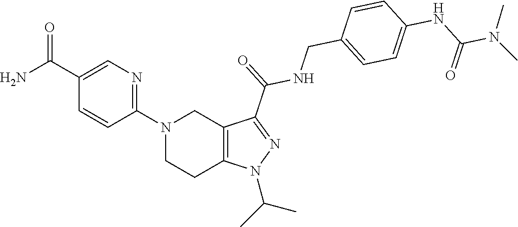 [SMILES: CC(C)n1nc(C(=O)NCc2ccc(NC(=O)N(C)C)cc2)c2c1CCN(c1ccc(C(N)=O)cn1)C2] ¹H NMR (400 MHz, (CD₃)₂SO) δ 8.62 (d, 1H), 8.34 (t, 1H), 8.23 (s, 1H), 7.97 (dd, 1H), 7.80-7.71 (m, 1H), 7.41-7.36 (m, 2H), 7.19-7.10 (m, 3H), 6.88 (d, 1H), 4.76 (s, 2H), 4.55-4.42 (m, 1H), 4.34 (d, 2H), 3.96 (t, 2H), 2.90 (s, 6H), 2.83-2.77 (m, 2H), 1.38 (d, 6H). | 5-(5-carbamoylpyridin-2- yl)-N-(4-(3,3- dimethylureido)benzyl)- 1-isopropyl-4,5,6,7- tetrahydro-1H- pyrazolo[4,3-c]pyridine- 3-carboxamide | 505.4 |
| 138 | Ex. 1 | 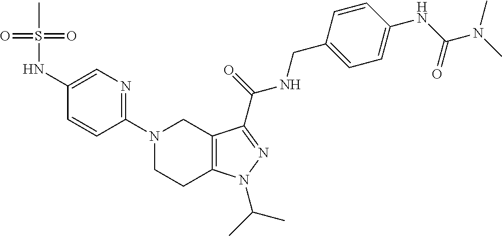 [SMILES: CC(C)n1nc(C(=O)NCc2ccc(NC(=O)N(C)C)cc2)c2c1CCN(c1ccc(NS(C)(=O)=O)cn1)C2] ¹H NMR (400 MHz, CD₃OD) δ 8.03 (d, 1H), 7.51 (dd, 1H), 7.35-7.30 (m, 2H), 7.28- 7.22 (m, 2H), 6.87 (d, 1H), 4.66 (s, 2H), 4.54-4.44 (m, 3H), 3.96 (t, 2H), 2.99 (s, 6H), 2.88 (s, 3H), 2.83 (t, 2H), 1.44 (d, 6H). | N-(4-(3,3- dimethylureido)benzyl)- 1-isopropyl-5-(5- (methylsulfonamido) pyridin-2-yl)-4,5,6,7- tetrahydro-1H- pyrazolo[4,3-c]pyridine- 3-carboxamide | 555.3 |
| 139 | Ex. 113 | 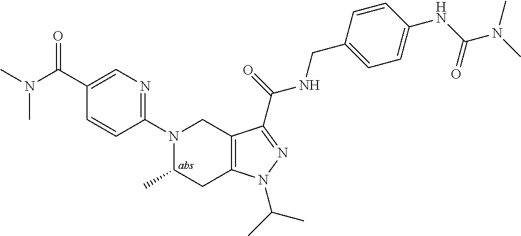 ¹H NMR (400 MHz, (CD₃)₂SO) δ 8.35 (t, 1H), 8.28 (d, 1H), 8.24 (s, 1H), 7.67 (dd, 1H), 7.44-7.38 (m, 2H), 7.23-7.19 (m, 2H), 6.86 (d, 1H), 5.33-5.23 (m, 1H), 5.15 (d, 1H), 4.59-4.49 (m, 1H), 4.36 (d, 2H), 4.12 (d, 1H), 3.03-2.95 (m, 7H), 2.92 (s, 6H), 2.81-2.74 (m, 1H), 1.42 (dd, 6H), 1.08 (d, 3H). | (S)-5-(5- (dimethylcarbamoyl) pyridin-2-yl)-N-(4-(3,3- dimethylureido)benzyl)- 1-isopropyl-6-methyl- 4,5,6,7-tetrahydro-1H- pyrazolo[4,3-c]pyridine- 3-carboxamide | 547.3 |
| 140 | Ex. 136 | 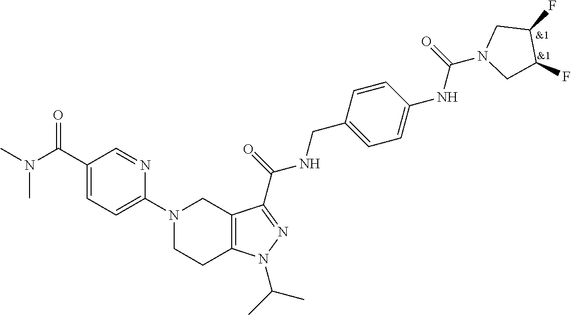 [SMILES: CC(C)n1nc(C(=O)NCc2ccc(NC(=O)N3C[C@@H](F)[C@@H](F)C3)cc2)c2c1CCN(c1ccc(C(=O)N(C)C)cn1)C2] | N-(4-((3R,4S)-3,4- difluoropyrrolidine-1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 595.5 |
| 141 | Ex. 136 | 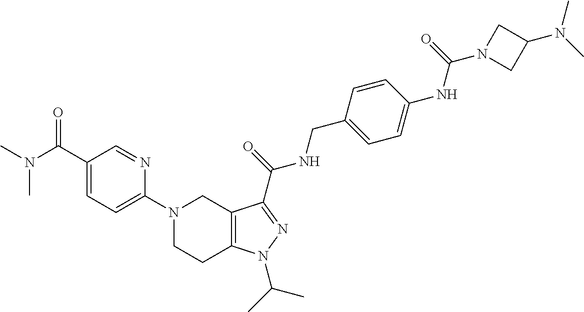 | N-(4-(3- (dimethylamino)azetidine- 1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 588.5 |
| 142 | Ex. 136 | 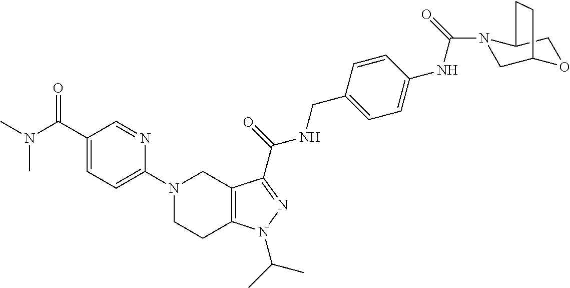 [SMILES: CC(C)n1nc(C(=O)NCc2ccc(NC(=O)N3CC4CCC3CO4)cc2)c2c1CCN(c1ccc(C(=O)N(C)C)cn1)C2] | (1S,4S)-N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)-2-oxa-5- azabicyclo[2.2.2]octane- 5-carboxamide | 601.5 |
| 143 | Ex. 136 | 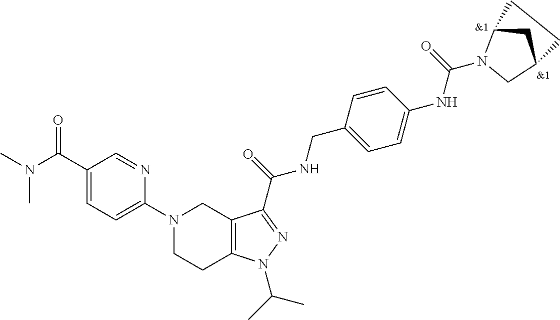 [SMILES: CC(C)n1nc(C(=O)NCc2ccc(NC(=O)N3C[C@H]4CC[C@@H]3C4)cc2)c2c1CCN(c1ccc(C(=O)N(C)C)cn1)C2] | rac-N-(4-((1R,4S)-2- azabicyclo[2.2.1]heptane- 2- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 585.6 |
| 144 | Ex. 136 | 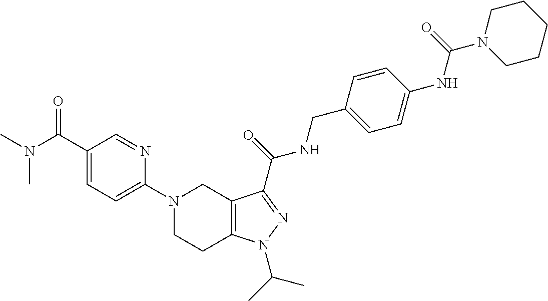 [SMILES: CC(C)n1nc(C(=O)NCc2ccc(NC(=O)N3CCCCC3)cc2)c2c1CCN(c1ccc(C(=O)N(C)C)cn1)C2] | 5-(5- (dimethylcarbamoyl) pyridin-2-yl)-1-isopropyl-N- (4-(piperidine-1- carboxamido)benzyl)- 4,5,6,7-tetrahydro-1H- pyrazolo[4,3-c]pyridine- 3-carboxamide | 573.5 |
| 145 | Ex. 136 | 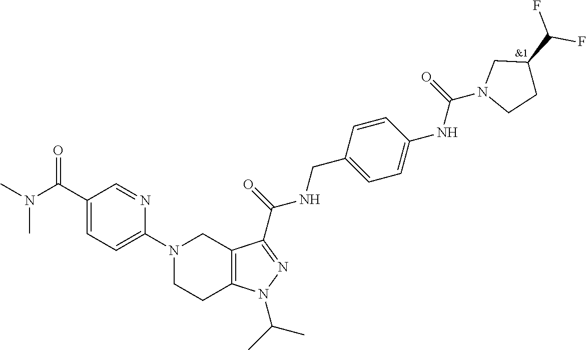 [SMILES: CC(C)n1nc(C(=O)NCc2ccc(NC(=O)N3CC[C@H](C(F)F)C3)cc2)c2c1CCN(c1ccc(C(=O)N(C)C)cn1)C2] | rac-(R)-N-(4-(3- (difluoromethyl)pyrrolidine- 1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 609.5 |
| 146 | Ex. 1 | 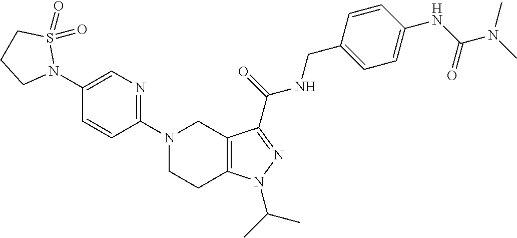 [SMILES: CC(C)n1nc(C(=O)NCc2ccc(NC(=O)N(C)C)cc2)c2c1CCN(c1ccc(N3CCCS3(=O)=O)cn1)C2] 1H NMR (400 MHz, (CD₃)₂SO) δ 8.29 (t, 1H), 8.20 (s, 1H), 7.99 (d, 1H), 7.50 (dd, 1H), 7.38-7.31 (m, 2H), 7.17-7.11 (m, 2H), 6.90 (d, 1H), 4.63 (s, 2H), 4.50-4.40 (m, 1H), 4.30 (d, 2H), 3.84 (t, 2H), 3.60 (t, 2H), 3.41-3.35 (m, 2H), 2.87 (s, 6H), 2.77 (t, 2H), 2.38-2.27 (m, 2H), 1.35 (d, 6H). | N-(4-(3,3- dimethylureido)benzyl)- 5-(5-(1,1- dioxidoisothiazolidin-2- yl)pyridin-2-yl)-1- isopropyl-4,5,6,7- tetrahydro-1H- pyrazolo[4,3-c]pyridine- 3-carboxamide | 581.3 |
| 147 | Ex. 113 | 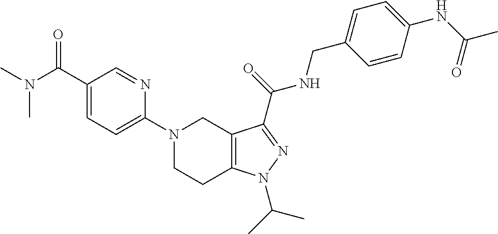 [SMILES: CC(=O)Nc1ccc(CNC(=O)c2nn(C(C)C)c3c2CN(c2ccc(C(=O)N(C)C)cn2)CC3)cc1] ¹H NMR (400 MHz, (CD₃)₂SO) δ 9.87 (s, 1H), 8.36 (t, 1H), 8.20 (d, 1H), 7.60 (dd, 1H), 7.50-7.42 (m, 2H), 7.20 (d, 2H), 6.85 (d, 1H), 4.71 (s, 2H), 4.51-4.37 (m, 1H), 4.32 (d, 2H), 3.91 (t, 2H), 2.93 (s, 6H), 2.77 (t, 2H), 1.98 (s, 3H), 1.36 (d, 6H). | N-(4-acetamidobenzyl)- 5-(5- (dimethylcarbamoyl) pyridin-2-yl)-1-isopropyl- 4,5,6,7-tetrahydro-1H- pyrazolo[4,3-c]pyridine- 3-carboxamide | 504.5 |
| 148 | Ex. 113 | 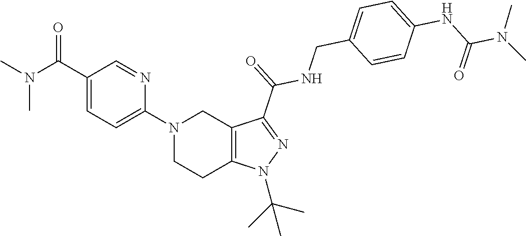 ¹H NMR (400 MHz, (CD₃)₂SO) δ 8.33 (t, 1H), 8.27-8.21 (m, 2H), 7.64 (dd, 1H), 7.41-7.36 (m, 2H), 7.17 (d, 2H), 6.87 (d, 1H), 4.74 (s, 2H), 4.35 (d, 2H), 3.94-3.88 (m, 2H), 3.04-2.99 (m, 2H), 2.97 (s, 6H), 2.90 (s, 6H), 1.57 (s, 9H). | 1-(tert-butyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 547.4 |
| 149 | Ex. 136 | 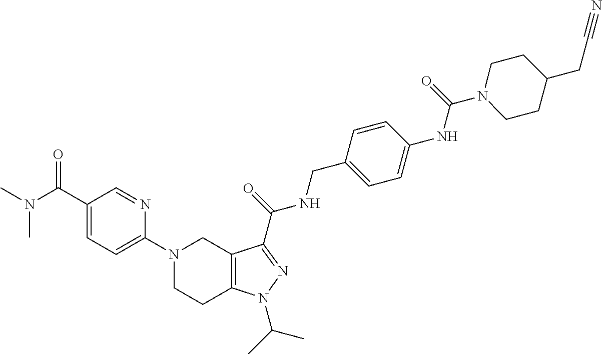 [SMILES: CC(C)n1nc(C(=O)NCc2ccc(NC(=O)N3CCC(CC#N)CC3)cc2)c2c1CCN(c1ccc(C(=O)N(C)C)cn1)C2] | N-(4-(4- (cyanomethyl)piperidine- 1-carboxamido)benzyl)- 5-(5- (dimethylcarbamoyl) pyridin-2-yl)-1-isopropyl- 4,5,6,7-tetrahydro-1H- pyrazolo[4,3-c]pyridine- 3-carboxamide | 612.6 |
| 150 | Ex. 136 | 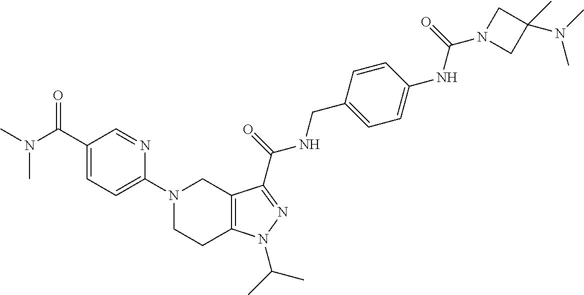 | N-(4-(3-(dimethylamino)- 3-methylazetidine-1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 602.5 |
| 151 | Ex. 136 | 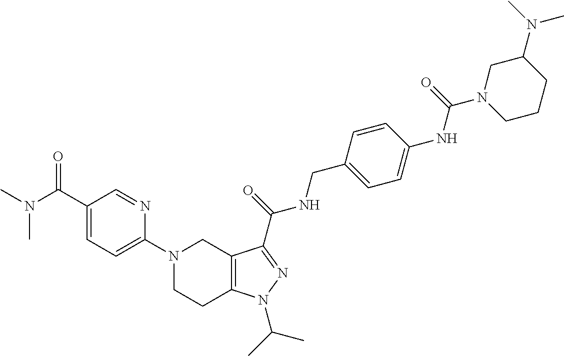 | N-(4-(3- (dimethylamino)piperidine- 1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 616.6 |
| 152 | Ex. 136 | 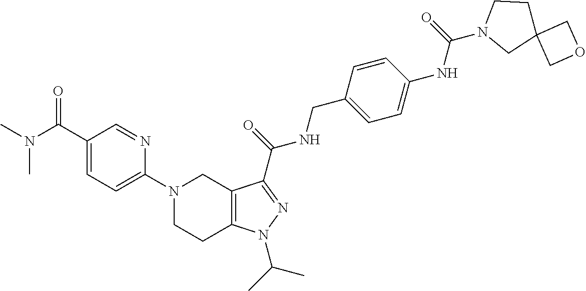 [SMILES: CC(C)n1nc(C(=O)NCc2ccc(NC(=O)N3CCC4(COC4)C3)cc2)c2c1CCN(c1ccc(C(=O)N(C)C)cn1)C2] | N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)-2-oxa-6- azaspiro[3.4]octane-6- carboxamide | 601.5 |
| 153 | Ex. 136 | 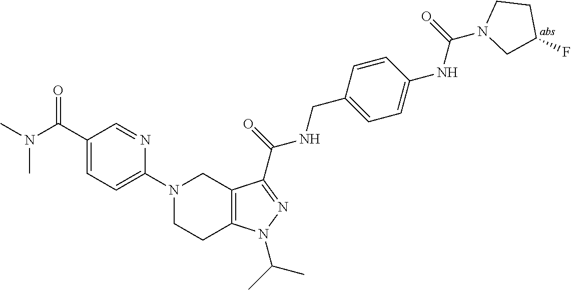 [SMILES: CC(C)n1nc(C(=O)NCc2ccc(NC(=O)N3CC[C@H](F)C3)cc2)c2c1CCN(c1ccc(C(=O)N(C)C)cn1)C2] | (S)-5-(5- (dimethylcarbamoyl) pyridin-2-yl)-N-(4-(3- fluoropyrrolidine-1- carboxamido)benzyl)-1- isopropyl-4,5,6,7- tetrahydro-1H- pyrazolo[4,3-c]pyridine- 3-carboxamide | 577.5 |
| 154 | Ex. 136 | 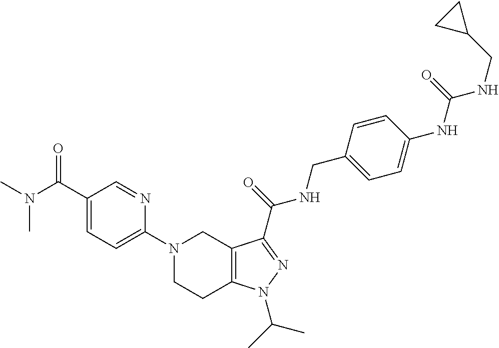 [SMILES: CC(C)n1nc(C(=O)NCc2ccc(NC(=O)NCC3CC3)cc2)c2c1CCN(c1ccc(C(=O)N(C)C)cn1)C2] | N-(4-(3- (cyclopropylmethyl)ureido) benzyl)-5-(5- (dimethylcarbamoyl) pyridin-2-yl)-1-isopropyl- 4,5,6,7-tetrahydro-1H- pyrazolo[4,3-c]pyridine- 3-carboxamide | 559.5 |
| 155 | Ex. 136 | 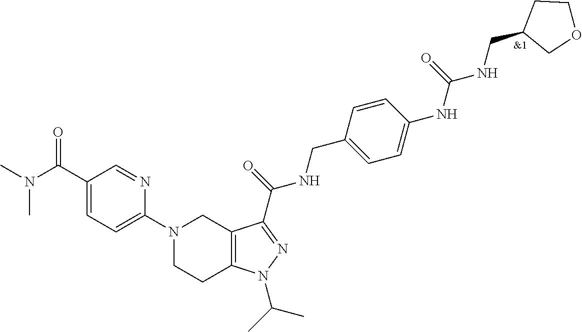 [SMILES: CC(C)n1nc(C(=O)NCc2ccc(NC(=O)NC[C@H]3CCOC3)cc2)c2c1CCN(c1ccc(C(=O)N(C)C)cn1)C2] | rac-(R)-5-(5- (dimethylcarbamoyl) pyridin-2-yl)-1-isopropyl-N- (4-(3-((tetrahydrofuran- 3- yl)methyl)ureido)benzyl)- 4,5,6,7-tetrahydro-1H- pyrazolo[4,3-c]pyridine- 3-carboxamide | 589.5 |
| 156 | Ex. 136 | 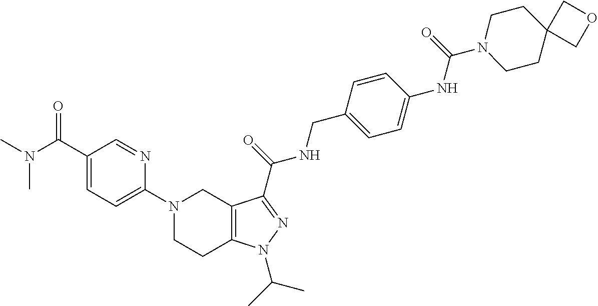 [SMILES: CC(C)n1nc(C(=O)NCc2ccc(NC(=O)N3CCC4(CC3)COC4)cc2)c2c1CCN(c1ccc(C(=O)N(C)C)cn1)C2] | N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)-2-oxa-7- azaspiro[3.5]nonane-7- carboxamide | 615.5 |
| 157 | Ex. 136 | 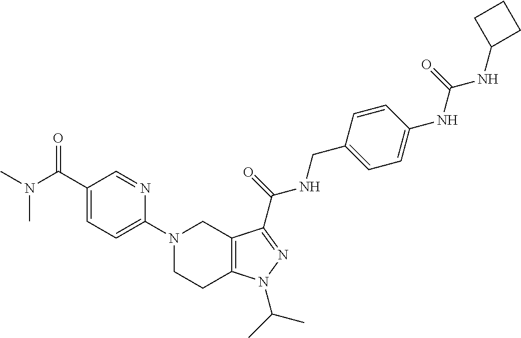 [SMILES: CC(C)n1nc(C(=O)NCc2ccc(NC(=O)NC3CCC3)cc2)c2c1CCN(c1ccc(C(=O)N(C)C)cn1)C2] | N-(4-(3- cyclobutylureido)benzyl)- 5-(5- (dimethylcarbamoyl) pyridin-2-yl)-1-isopropyl- 4,5,6,7-tetrahydro-1H- pyrazolo[4,3-c]pyridine- 3-carboxamide | 559.5 |
| 158 | Ex. 136 | 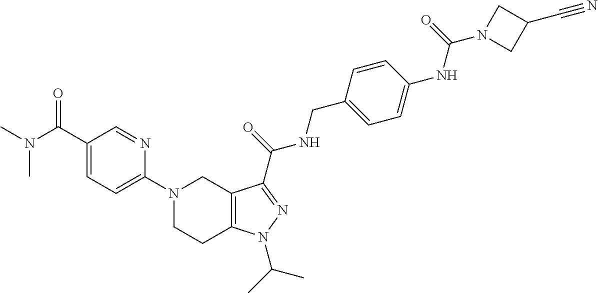 [SMILES: CC(C)n1nc(C(=O)NCc2ccc(NC(=O)N3CC(C#N)C3)cc2)c2c1CCN(c1ccc(C(=O)N(C)C)cn1)C2] | N-(4-(3-cyanoazetidine- 1-carboxamido)benzyl)- 5-(5- (dimethylcarbamoyl) pyridin-2-yl)-1-isopropyl- 4,5,6,7-tetrahydro-1H- pyrazolo[4,3-c]pyridine- 3-carboxamide | 570.5 |
| 159 | Ex. 136 | 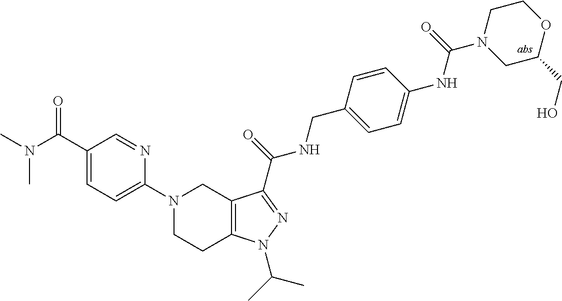 [SMILES: CC(C)n1nc(C(=O)NCc2ccc(NC(=O)N3CCO[C@H](CO)C3)cc2)c2c1CCN(c1ccc(C(=O)N(C)C)cn1)C2] | (S)-N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)-2- (hydroxymethyl)morpholine- 4-carboxamide | 605.5 |
| 160 | Ex. 136 | 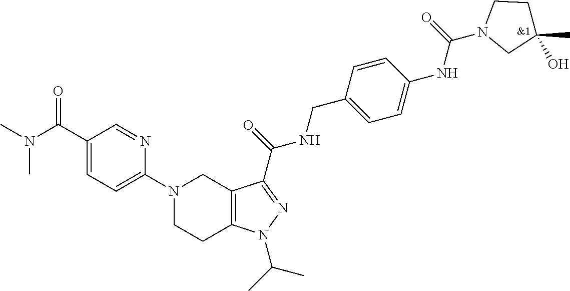 | rac-(R)-5-(5- (dimethylcarbamoyl) pyridin-2-yl)-N-(4-(3- hydroxy-3- methylpyrrolidine-1- carboxamido)benzyl)-1- isopropyl-4,5,6,7- tetrahydro-1H- pyrazolo[4,3-c]pyridine- 3-carboxamide | 589.5 |
| 161 | Ex. 136 | 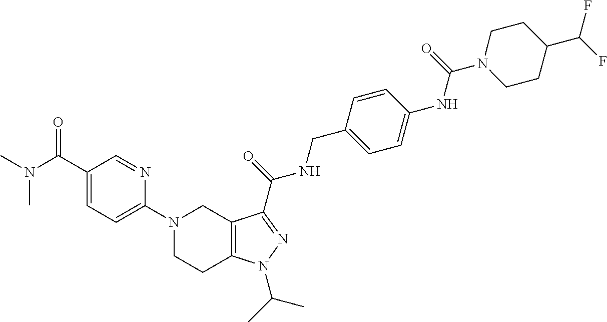 [SMILES: CC(C)n1nc(C(=O)NCc2ccc(NC(=O)N3CCC(C(F)F)CC3)cc2)c2c1CCN(c1ccc(C(=O)N(C)C)cn1)C2] | N-(4-(4- (difluoromethyl)piperidine- 1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 623.5 |
| 162 | Ex. 136 | 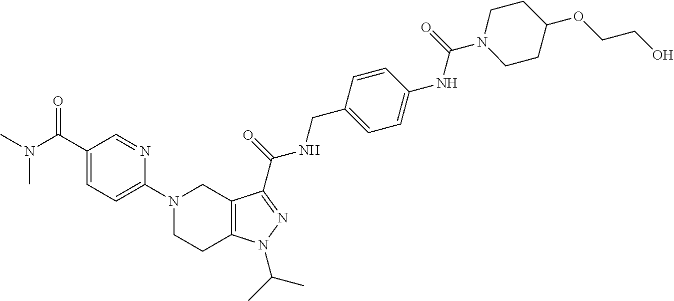 [SMILES: CC(C)n1nc(C(=O)NCc2ccc(NC(=O)N3CCC(OCCO)CC3)cc2)c2c1CCN(c1ccc(C(=O)N(C)C)cn1)C2] | 5-(5- (dimethylcarbamoyl) pyridin-2-yl)-N-(4-(4-(2- hydroxyethoxy)piperidine- 1- carboxamido)benzyl)-1- isopropyl-4,5,6,7- tetrahydro-1H- pyrazolo[4,3-c]pyridine- 3-carboxamide | 633.5 |
| 163 | Ex. 136 | 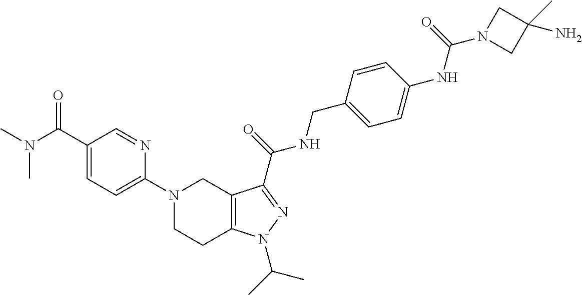 [SMILES: CC(C)n1nc(C(=O)NCc2ccc(NC(=O)N3CC(C)(N)C3)cc2)c2c1CCN(c1ccc(C(=O)N(C)C)cn1)C2] | N-(4-(3-amino-3- methylazetidine-1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 574.5 |
| 164 | Ex. 136 | 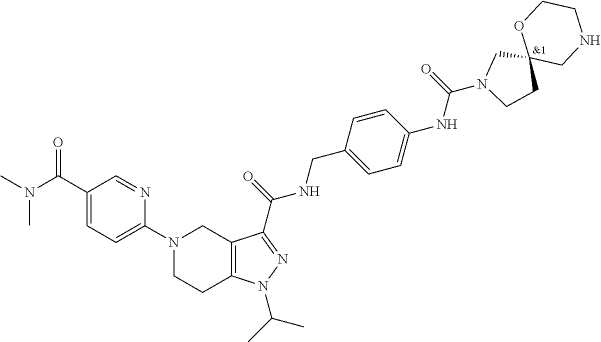 [SMILES: CC(C)n1nc(C(=O)NCc2ccc(NC(=O)N3CC[C@@]4(CNCCO4)C3)cc2)c2c1CCN(c1ccc(C(=O)N(C)C)cn1)C2] | rac-(R)-N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)-6-oxa-2,9- diazaspiro[4.5]decane- 2-carboxamide | 630.6 |
| 165 | Ex. 136 | 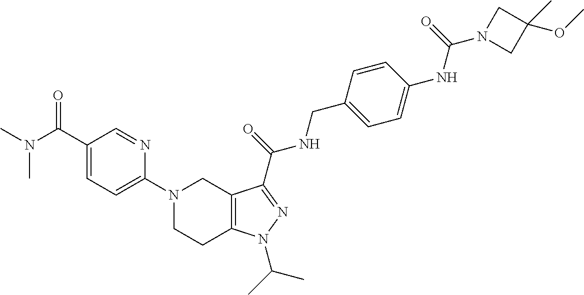 | 5-(5- (dimethylcarbamoyl) pyridin-2-yl)-1-isopropyl-N- (4-(3-methoxy-3- methylazetidine-1- carboxamido)benzyl)- 4,5,6,7-tetrahydro-1H- pyrazolo[4,3-c]pyridine- 3-carboxamide | 589.4 |
| 166 | Ex. 136 | 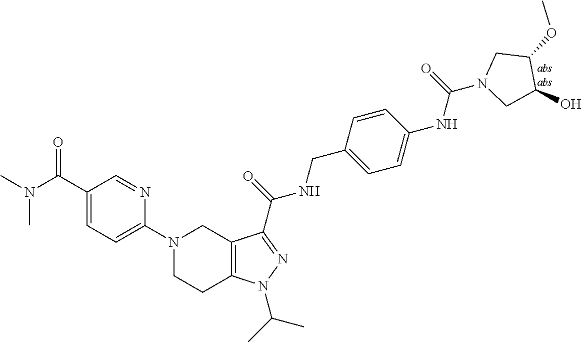 [SMILES: CO[C@H]1CN(C(=O)Nc2ccc(CNC(=O)c3nn(C(C)C)c4c3CN(c3ccc(C(=O)N(C)C)cn3)CC4)cc2)C[C@@H]1O] | 5-(5- (dimethylcarbamoyl) pyridin-2-yl)-N-(4-((3S,4S)- 3-hydroxy-4- methoxypyrrolidine-1- carboxamido)benzyl)-1- isopropyl-4,5,6,7- tetrahydro-1H- pyrazolo[4,3-c]pyridine- 3-carboxamide | 605.4 |
| 167 | Ex. 136 | 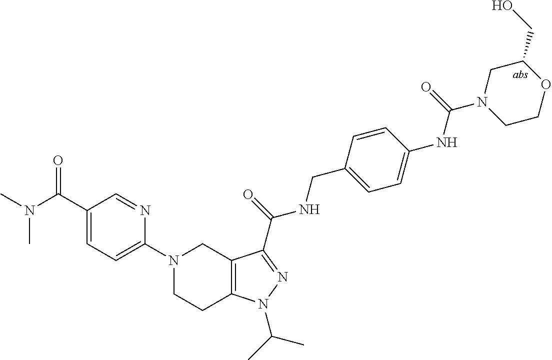 [SMILES: CC(C)n1nc(C(=O)NCc2ccc(NC(=O)N3CCO[C@@H](CO)C3)cc2)c2c1CCN(c1ccc(C(=O)N(C)C)cn1)C2] | (R)-N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)-2- (hydroxymethyl)morpholine- 4-carboxamide | 605.4 |
| 168 | Ex. 136 | 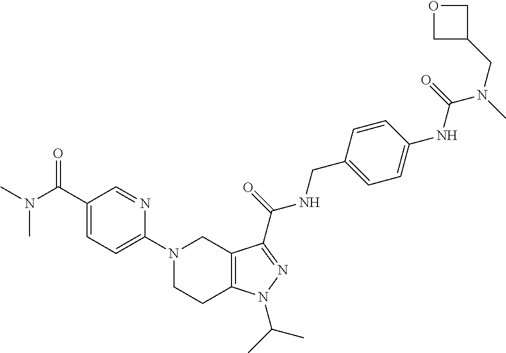 | 5-(5- (dimethylcarbamoyl) pyridin-2-yl)-1-isopropyl-N- (4-(3-methyl-3-(oxetan- 3- ylmethyl)ureido)benzyl)- 4,5,6,7-tetrahydro-1H- pyrazolo[4,3-c]pyridine- 3-carboxamide | 589.4 |
| 169 | Ex. 136 | 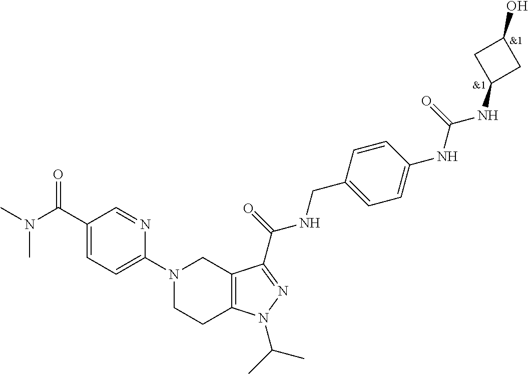 [SMILES: CC(C)n1nc(C(=O)NCc2ccc(NC(=O)N[C@H]3C[C@@H](O)C3)cc2)c2c1CCN(c1ccc(C(=O)N(C)C)cn1)C2] | 5-(5- (dimethylcarbamoyl) pyridin-2-yl)-N-(4-(3- ((1s,3s)-3- hydroxycyclobutyl)ureido) benzyl)-1-isopropyl- 4,5,6,7-tetrahydro-1H- pyrazolo[4,3-c]pyridine- 3-carboxamide | 575.4 |
| 170 | Ex. 136 | 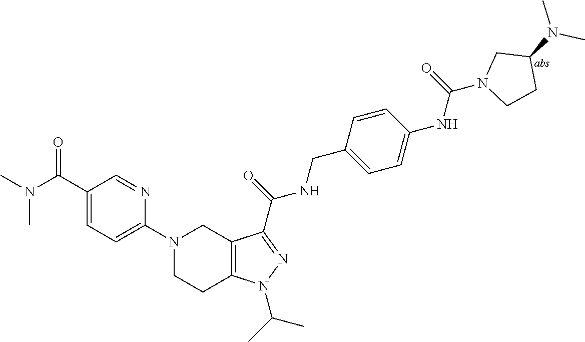 | (S)-N-(4-(3- (dimethylamino)pyrrolidine- 1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 602.5 |
| 171 | Ex. 136 | 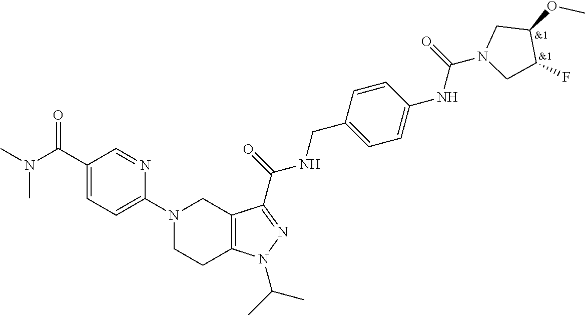 | rac-5-(5- (dimethylcarbamoyl) pyridin-2-yl)-N-(4-((3R,4R)- 3-fluoro-4- methoxypyrrolidine-1- carboxamido)benzyl)-1- isopropyl-4,5,6,7- tetrahydro-1H- pyrazolo[4,3-c]pyridine- 3-carboxamide | 607.4 |
| 172 | Ex. 136 | 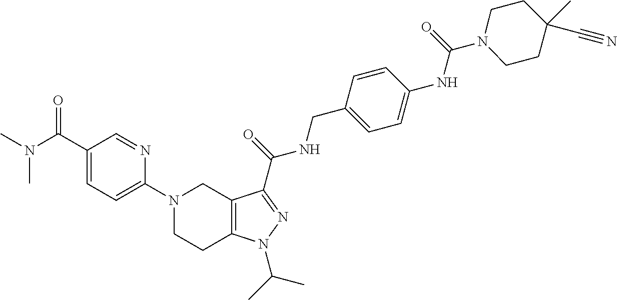 [SMILES: CC(C)n1nc(C(=O)NCc2ccc(NC(=O)N3CCC(C)(C#N)CC3)cc2)c2c1CCN(c1ccc(C(=O)N(C)C)cn1)C2] | N-(4-(4-cyano-4- methylpiperidine-1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 612.4 |
| 173 | Ex. 136 | 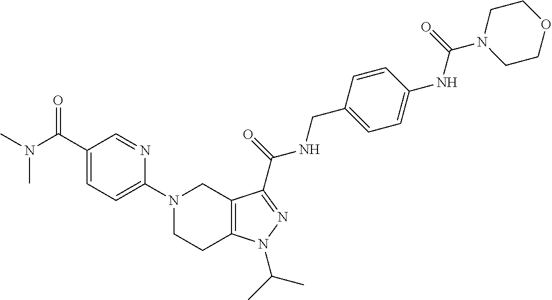 [SMILES: CC(C)n1nc(C(=O)NCc2ccc(NC(=O)N3CCOCC3)cc2)c2c1CCN(c1ccc(C(=O)N(C)C)cn1)C2] | N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)morpholine-4- carboxamide | 575.4 |
| 174 | Ex. 136 | 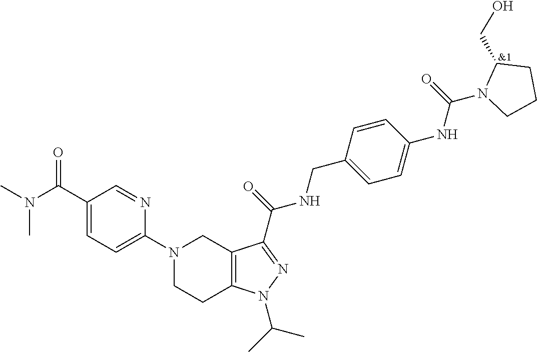 [SMILES: CC(C)n1nc(C(=O)NCc2ccc(NC(=O)N3CCC[C@H]3CO)cc2)c2c1CCN(c1ccc(C(=O)N(C)C)cn1)C2] | rac-(R)-5-(5- (dimethylcarbamoyl) pyridin-2-yl)-N-(4-(2- (hydroxymethyl)pyrrolidine- 1- carboxamido)benzyl)-1- isopropyl-4,5,6,7- tetrahydro-1H- pyrazolo[4,3-c]pyridine- 3-carboxamide | 589.4 |
| 175 | Ex. 136 | 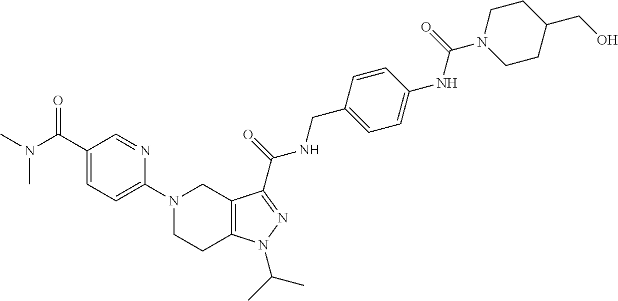 [SMILES: CC(C)n1nc(C(=O)NCc2ccc(NC(=O)N3CCC(CO)CC3)cc2)c2c1CCN(c1ccc(C(=O)N(C)C)cn1)C2] | 5-(5- (dimethylcarbamoyl) pyridin-2-yl)-N-(4-(4- (hydroxymethyl)piperidine- 1- carboxamido)benzyl)-1- isopropyl-4,5,6,7- tetrahydro-1H- pyrazolo[4,3-c]pyridine- 3-carboxamide | 603.4 |
| 176 | Ex. 136 | 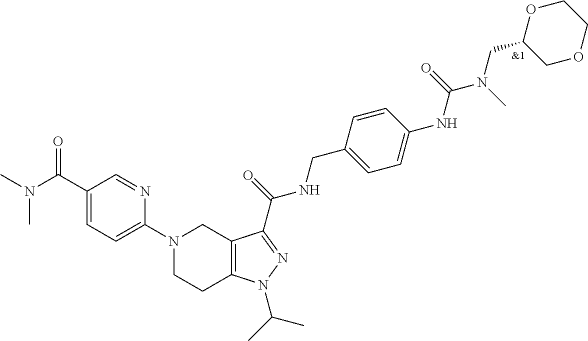 [SMILES: CC(C)n1nc(C(=O)NCc2ccc(NC(=O)N(C)C[C@H]3COCCO3)cc2)c2c1CCN(c1ccc(C(=O)N(C)C)cn1)C2] | rac-(R)-N-(4-(3-((1,4- dioxan-2-yl)methyl)-3- methylureido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 619.4 |
| 177 | Ex. 136 | 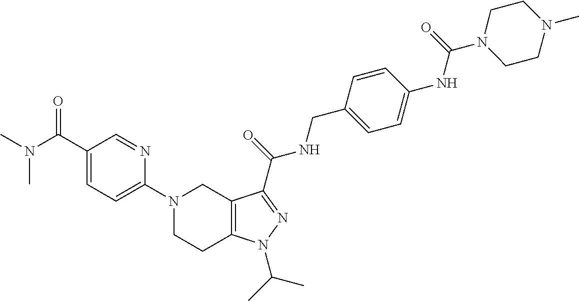 | 5-(5- (dimethylcarbamoyl) pyridin-2-yl)-1-isopropyl-N- (4-(4-methylpiperazine- 1-carboxamido)benzyl)- 4,5,6,7-tetrahydro-1H- pyrazolo[4,3-c]pyridine- 3-carboxamide | 588.5 |
| 178 | Ex. 136 | 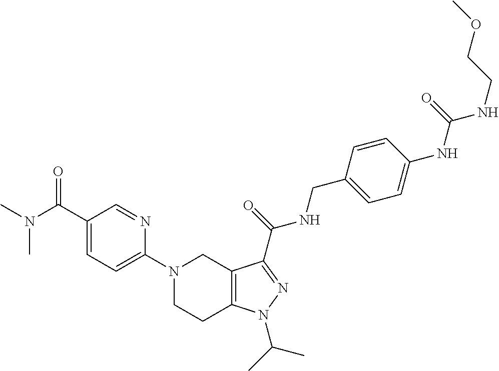 [SMILES: COCCNC(=O)Nc1ccc(CNC(=O)c2nn(C(C)C)c3c2CN(c2ccc(C(=O)N(C)C)cn2)CC3)cc1] | 5-(5- (dimethylcarbamoyl) pyridin-2-yl)-1-isopropyl-N- (4-(3-(2- methoxyethyl)ureido) benzyl)-4,5,6,7-tetrahydro- 1H-pyrazolo[4,3- c]pyridine-3- carboxamide | 563.4 |
| 179 | Ex. 136 | 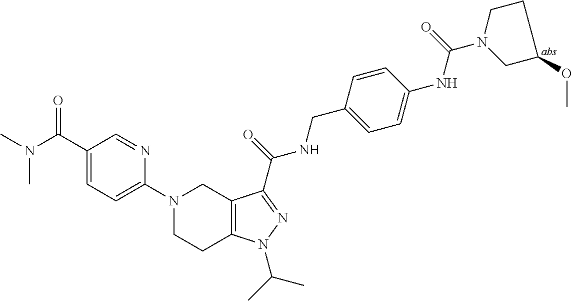 | (R)-5-(5- (dimethylcarbamoyl) pyridin-2-yl)-1-isopropyl-N- (4-(3- methoxypyrrolidine-1- carboxamido)benzyl)- 4,5,6,7-tetrahydro-1H- pyrazolo[4,3-c]pyridine- 3-carboxamide | 589.4 |
| 180 | Ex. 136 | 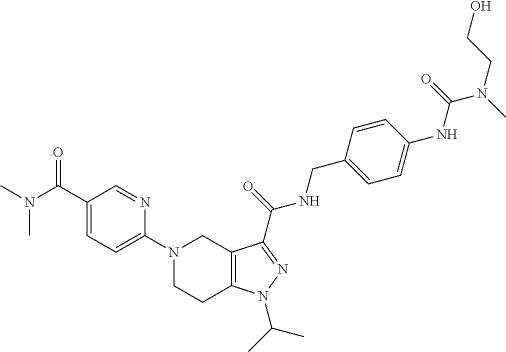 | 5-(5- (dimethylcarbamoyl) pyridin-2-yl)-N-(4-(3-(2- hydroxyethyl)-3- methylureido)benzyl)-1- isopropyl-4,5,6,7- tetrahydro-1H- pyrazolo[4,3-c]pyridine- 3-carboxamide | 563.4 |
| 181 | Ex. 136 | 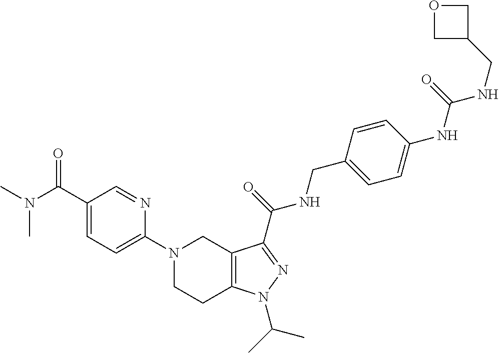 [SMILES: CC(C)n1nc(C(=O)NCc2ccc(NC(=O)NCC3COC3)cc2)c2c1CCN(c1ccc(C(=O)N(C)C)cn1)C2] | 5-(5- (dimethylcarbamoyl) pyridin-2-yl)-1-isopropyl-N- (4-(3-(oxetan-3- ylmethyl)ureido)benzyl)- 4,5,6,7-tetrahydro-1H- pyrazolo[4,3-c]pyridine- 3-carboxamide | 575.4 |
| 182 | Ex. 136 | 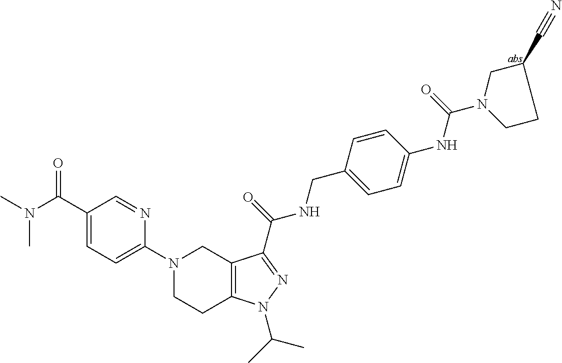 [SMILES: CC(C)n1nc(C(=O)NCc2ccc(NC(=O)N3CC[C@H](C#N)C3)cc2)c2c1CCN(c1ccc(C(=O)N(C)C)cn1)C2] | (S)-N-(4-(3- cyanopyrrolidine-1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 584.4 |
| 183 | Ex. 136 | 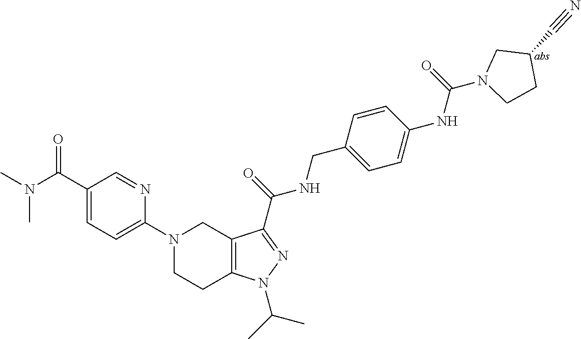 [SMILES: CC(C)n1nc(C(=O)NCc2ccc(NC(=O)N3CC[C@@H](C#N)C3)cc2)c2c1CCN(c1ccc(C(=O)N(C)C)cn1)C2] | (R)-N-(4-(3- cyanopyrrolidine-1- carboxamido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 584.4 |
| 184 | Ex. 136 | 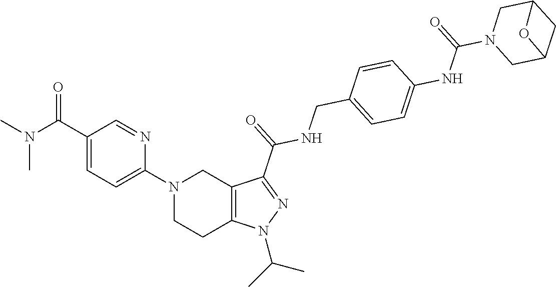 [SMILES: CC(C)n1nc(C(=O)NCc2ccc(NC(=O)N3CC4CC(C3)O4)cc2)c2c1CCN(c1ccc(C(=O)N(C)C)cn1)C2] | N-(4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)-6-oxa-3- azabicyclo[3.1.1]heptane- 3-carboxamide | 587.4 |
| 185 | Ex. 136 | 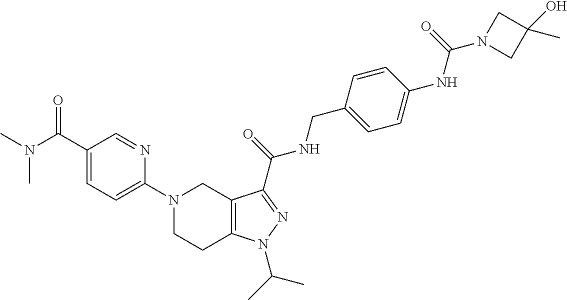 | 5-(5- (dimethylcarbamoyl) pyridin-2-yl)-N-(4-(3- hydroxy-3- methylazetidine-1- carboxamido)benzyl)-1- isopropyl-4,5,6,7- tetrahydro-1H- pyrazolo[4,3-c]pyridine- 3-carboxamide | 575.4 |
| 186 | Ex. 136 | 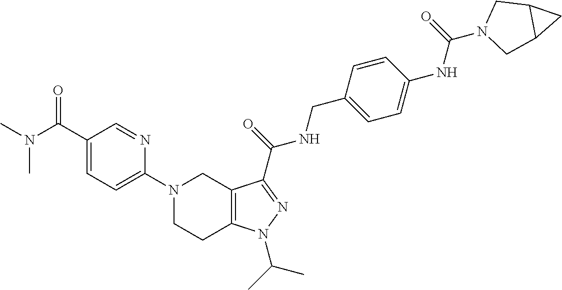 [SMILES: CC(C)n1nc(C(=O)NCc2ccc(NC(=O)N3CC4CC4C3)cc2)c2c1CCN(c1ccc(C(=O)N(C)C)cn1)C2] | N-(4-(3- azabicyclo[3.1.0]hexane- 3-carboxamido)benzyl)- 5-(5- (dimethylcarbamoyl) pyridin-2-yl)-1-isopropyl- 4,5,6,7-tetrahydro-1H- pyrazolo[4,3-c]pyridine- 3-carboxamide | 571.4 |
| 187 | Ex. 136 | 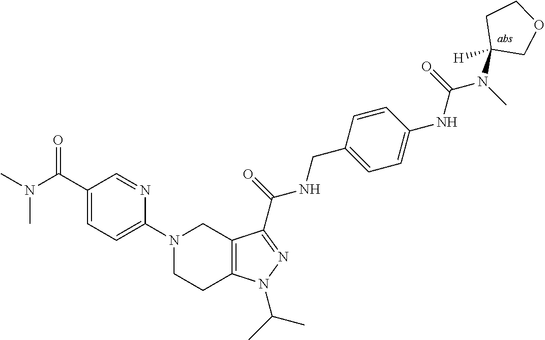 [SMILES: CC(C)n1nc(C(=O)NCc2ccc(NC(=O)N(C)[C@H]3CCOC3)cc2)c2c1CCN(c1ccc(C(=O)N(C)C)cn1)C2] | (S)-5-(5- (dimethylcarbamoyl) pyridin-2-yl)-1-isopropyl-N- (4-(3-methyl-3- (tetrahydrofuran-3- yl)ureido)benzyl)- 4,5,6,7-tetrahydro-1H- pyrazolo[4,3-c]pyridine- 3-carboxamide | 589.4 |
| 188 | Ex. 136 | 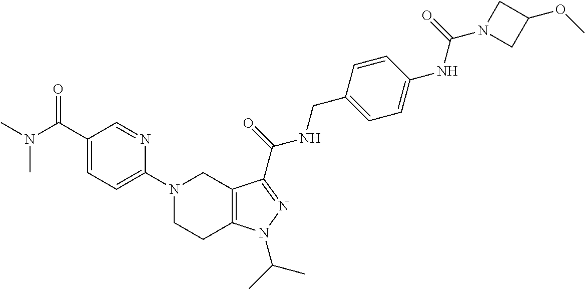 | 5-(5- (dimethylcarbamoyl) pyridin-2-yl)-1-isopropyl-N- (4-(3-methoxyazetidine- 1-carboxamido)benzyl)- 4,5,6,7-tetrahydro-1H- pyrazolo[4,3-c]pyridine- 3-carboxamide | 575.4 |
| 189 | Ex. 136 | 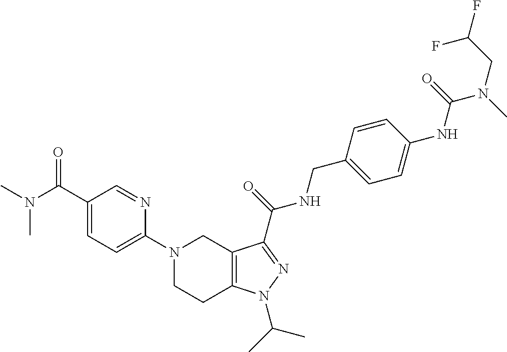 | N-(4-(3-(2,2- difluoroethyl)-3- methylureido)benzyl)-5- (5- (dimethylcarbamoyl) pyridin-2-yl)-1-isopropyl- 4,5,6,7-tetrahydro-1H- pyrazolo[4,3-c]pyridine- 3-carboxamide | 583.4 |
| 190^{∧} | Ex. 5 | 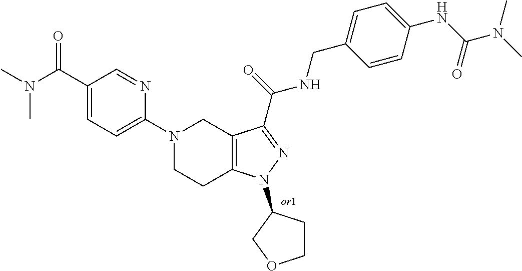 ¹H NMR (400 MHz, (CD₃)₂SO) δ 8.41 (t, 1H), 8.24-8.19 (m, 2H), 7.62 (dd, 1H), 7.40-7.34 (m, 2H), 7.17-7.12 (m, 2H), 6.88 (d, 1H), 5.04-4.90 (m, 1H), 4.74 (s, 2H), 4.32 (d, 2H), 4.09-3.74 (m, 6H), 2.95 (s, 6H), 2.89 (s, 6H), 2.84-2.78 (m, 2H), 2.33-2.24 (m, 2H). | rel-(R or S)-5-(5- (dimethylcarbamoyl) pyridin-2-yl)-N-(4-(3,3- dimethylureido)benzyl)- 1-(tetrahydrofuran-3-yl)- 4,5,6,7-tetrahydro-1H- pyrazolo[4,3-c]pyridine- 3-carboxamide | 561.3 |
| 191^{∧} | Ex. 5 | 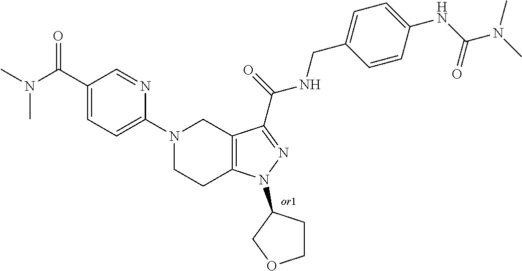 ¹H NMR (400 MHz, (CD₃)₂SO) δ 8.44- 8.39 (m, 1H), 8.24-8.21 (m, 2H), 7.63 (dd, 1H), 7.41-7.36 (m, 2H), 7.16 (d, 2H), 6.89 (d, 1H), 5.06-4.93 (m, 1H), 4.75 (s, 2H), 4.34 (d, 2H), 4.13-3.75 (m, 6H), 2.96 (s, 6H), 2.90 (s, 6H), 2.85-2.80 (m, 2H), 2.34- 2.25 (m, 2H). | rel-(R or S)-5-(5- (dimethylcarbamoyl) pyridin-2-yl)-N-(4-(3,3- dimethylureido)benzyl)- 1-(tetrahydrofuran-3-yl)- 4,5,6,7-tetrahydro-1H- pyrazolo[4,3-c]pyridine- 3-carboxamide | 561.5 |
| 192 | Ex. 12^{h} | 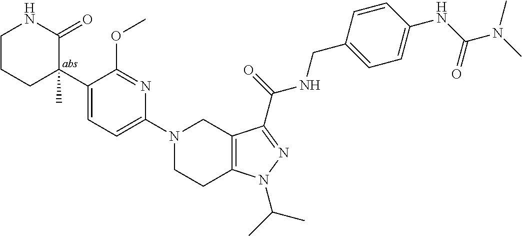 [SMILES: COc1nc(N2CCc3c(c(C(=O)NCc4ccc(NC(=O)N(C)C)cc4)nn3C(C)C)C2)ccc1[C@@]1(C)CCCNC1=O] ¹H NMR* (600 MHz, (CD₃)₂SO) δ 8.29 (t, 1H), 8.23 (s, 1H), 7.41-7.36 (m, 3H), 7.31 (d, 1H), 7.20-7.15 (m, 2H), 6.30 (d, 1H), 4.64-4.56 (m, 2H), 4.52-4.45 (m, 1H), 4.34 (d, 2H), 3.87 (t, 2H), 3.79 (s, 3H), 3.26- 3.12 (m, 2H), 2.91-2.89 (m, 6H), 2.81 (t, 2H), 2.19-2.10 (m, 1H), 1.81-1.72 (m, 1H), 1.64-1.56 (m, 1H), 1.52-1.45 (m, 1H), 1.43 (s, 3H), 1.39 (d, 6H). | (R)-N-(4-(3,3- dimethylureido)benzyl)- 1-isopropyl-5-(6- methoxy-5-(3-methyl-2- oxopiperidin-3-yl)pyridin- 2-yl)-4,5,6,7-tetrahydro- 1H-pyrazolo[4,3- c]pyridine-3- carboxamide | 603.4 |
| 193 | Ex. 193 | 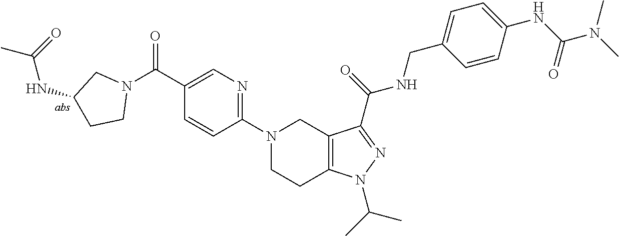 [SMILES: CC(=O)N[C@H]1CCN(C(=O)c2ccc(N3CCc4c(c(C(=O)NCc5ccc(NC(=O)N(C)C)cc5)nn4C(C)C)C3)nc2)C1] | (S)-5-(5-(3- acetamidopyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 616.4 |
| 194 | Ex. 193 | 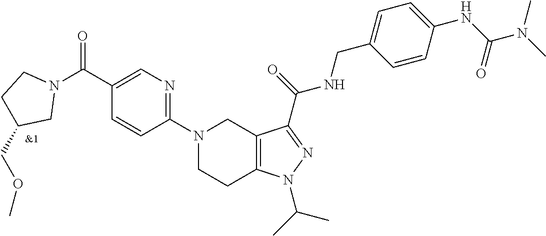 [SMILES: COC[C@@H]1CCN(C(=O)c2ccc(N3CCc4c(c(C(=O)NCc5ccc(NC(=O)N(C)C)cc5)nn4C(C)C)C3)nc2)C1] | rac-(R)-N-(4-(3,3- dimethylureido)benzyl)- 1-isopropyl-5-(5-(3- (methoxymethyl)pyrrolidine- 1-carbonyl)pyridin-2- yl)-4,5,6,7-tetrahydro- 1H-pyrazolo[4,3- c]pyridine-3- carboxamide | 603.9 |
| 195 | Ex. 193 | 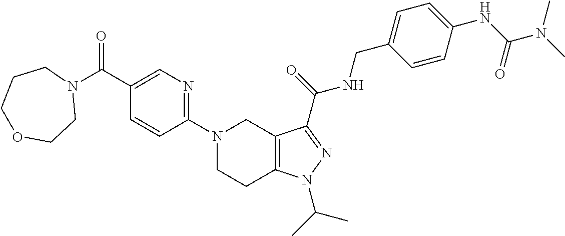 [SMILES: CC(C)n1nc(C(=O)NCc2ccc(NC(=O)N(C)C)cc2)c2c1CCN(c1ccc(C(=O)N3CCCOCC3)cn1)C2] | 5-(5-(1,4-oxazepane-4- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 589.8 |
| 196 | Ex. 193 | 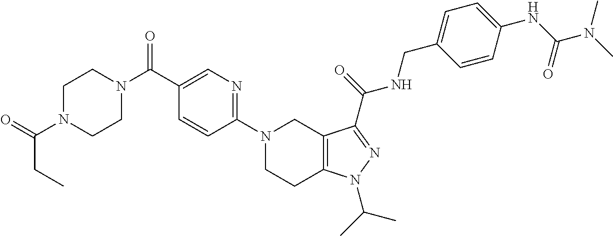 [SMILES: CCC(=O)N1CCN(C(=O)c2ccc(N3CCc4c(c(C(=O)NCc5ccc(NC(=O)N(C)C)cc5)nn4C(C)C)C3)nc2)CC1] | N-(4-(3,3- dimethylureido)benzyl)- 1-isopropyl-5-(5-(4- propionylpiperazine-1- carbonyl)pyridin-2-yl)- 4,5,6,7-tetrahydro-1H- pyrazolo[4,3-c]pyridine- 3-carboxamide | 630.8 |
| 197 | Ex. 193 | 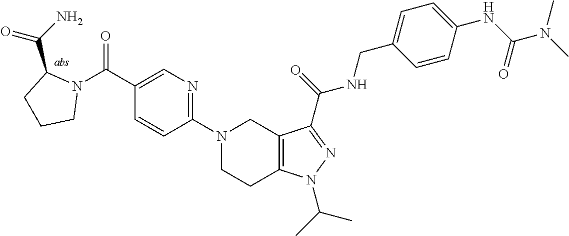 [SMILES: CC(C)n1nc(C(=O)NCc2ccc(NC(=O)N(C)C)cc2)c2c1CCN(c1ccc(C(=O)N3CCC[C@H]3C(N)=O)cn1)C2] | (S)-5-(5-(2- carbamoylpyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 602.4 |
| 198 | Ex. 193 | 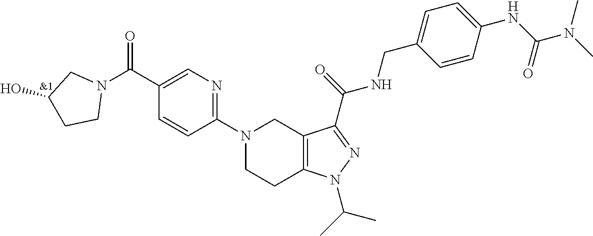 [SMILES: CC(C)n1nc(C(=O)NCc2ccc(NC(=O)N(C)C)cc2)c2c1CCN(c1ccc(C(=O)N3CC[C@H](O)C3)cn1)C2] | rac-(R)-N-(4-(3,3- dimethylureido)benzyl)- 5-(5-(3- hydroxypyrrolidine-1- carbonyl)pyridin-2-yl)-1- isopropyl-4,5,6,7- tetrahydro-1H- pyrazolo[4,3-c]pyridine- 3-carboxamide | 575.4 |
| 199 | Ex. 193 | 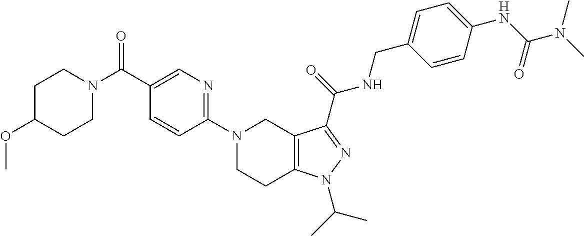 [SMILES: COC1CCN(C(=O)c2ccc(N3CCc4c(c(C(=O)NCc5ccc(NC(=O)N(C)C)cc5)nn4C(C)C)C3)nc2)CC1] | N-(4-(3,3- dimethylureido)benzyl)- 1-isopropyl-5-(5-(4- methoxypiperidine-1- carbonyl)pyridin-2-yl)- 4,5,6,7-tetrahydro-1H- pyrazolo[4,3-c]pyridine- 3-carboxamide | 603.4 |
| 200 | Ex. 193 | 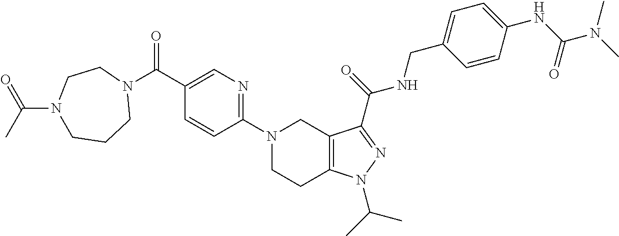 [SMILES: CC(=O)N1CCCN(C(=O)c2ccc(N3CCc4c(c(C(=O)NCc5ccc(NC(=O)N(C)C)cc5)nn4C(C)C)C3)nc2)CC1] | 5-(5-(4-acetyl-1,4- diazepane-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 630.4 |
| 201 | Ex. 193 | 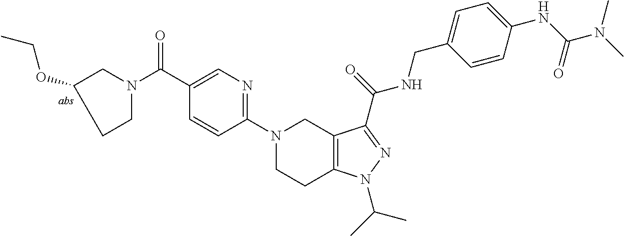 [SMILES: CCO[C@H]1CCN(C(=O)c2ccc(N3CCc4c(c(C(=O)NCc5ccc(NC(=O)N(C)C)cc5)nn4C(C)C)C3)nc2)C1] | (S)-N-(4-(3,3- dimethylureido)benzyl)- 5-(5-(3- ethoxypyrrolidine-1- carbonyl)pyridin-2-yl)-1- isopropyl-4,5,6,7- tetrahydro-1H- pyrazolo[4,3-c]pyridine- 3-carboxamide | 603.4 |
| 202 | Ex. 132^{r} | 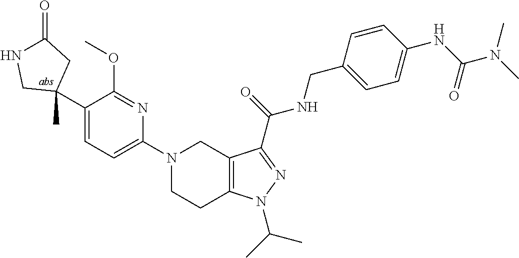 [SMILES: COc1nc(N2CCc3c(c(C(=O)NCc4ccc(NC(=O)N(C)C)cc4)nn3C(C)C)C2)ccc1[C@]1(C)CNC(=O)C1] | (R)-N-(4-(3,3- dimethylureido)benzyl)- 1-isopropyl-5-(6- methoxy-5-(3-methyl-5- oxopyrrolidin-3- yl)pyridin-2-yl)-4,5,6,7- tetrahydro-1H- pyrazolo[4,3-c]pyridine- 3-carboxamide | 589.5 |
| 203^{∧} | Ex. 42 | 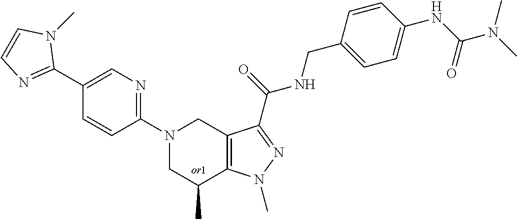 [SMILES: C[C@H]1CN(c2ccc(-c3nccn3C)cn2)Cc2c(C(=O)NCc3ccc(NC(=O)N(C)C)cc3)nn(C)c21] ¹H NMR (400 MHz, (CD₃)₂SO) δ 8.45 (t, 1H), 8.38 (d, 1H), 8.18 (s, 1H), 7.80 (dd, 1H), 7.35-7.28 (m, 2H), 7.15 (d, 1H), 7.13- 7.09 (m, 2H), 6.92 (d, 1H), 6.88 (d, 1H), 4.98 (d, 1H), 4.37-4.20 (m, 4H), 3.78 (s, 3H), 3.66 (s, 3H), 3.41-3.38 (m, 1H), 3.19- 3.11 (m, 1H), 2.85 (s, 6H), 1.18 (d, 3H). | rel-(R or S)-N-(4-(3,3- dimethylureido)benzyl)- 1,7-dimethyl-5-(5-(1- methyl-1H-imidazol-2- yl)pyridin-2-yl)-4,5,6,7- tetrahydro-1H- pyrazolo[4,3-c]pyridine- 3-carboxamide | 528.4 |
| 204^{∧} | Ex. 42 | 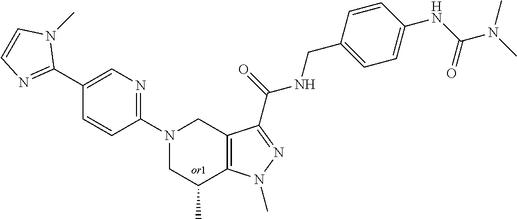 [SMILES: C[C@@H]1CN(c2ccc(-c3nccn3C)cn2)Cc2c(C(=O)NCc3ccc(NC(=O)N(C)C)cc3)nn(C)c21] ¹H NMR (400 MHz, (CD₃)₂SO) δ 8.46 (t, 1H), 8.39 (d, 1H), 8.19 (s, 1H), 7.81 (dd, 1H), 7.36-7.31 (m, 2H), 7.16 (d, 1H), 7.14- 7.10 (m, 2H), 6.93 (d, 1H), 6.89 (d, 1H), 4.99 (d, 1H), 4.37-4.22 (m, 4H), 3.79 (s, 3H), 3.67 (s, 3H), 3.44-3.37 (m, 1H), 3.24- 3.14 (m, 1H), 2.86 (s, 6H), 1.19 (d, 3H). | rel-(R or S)-N-(4-(3,3- dimethylureido)benzyl)- 1,7-dimethyl-5-(5-(1- methyl-1H-imidazol-2- yl)pyridin-2-yl)-4,5,6,7- tetrahydro-1H- pyrazolo[4,3-c]pyridine- 3-carboxamide | 528.4 |
| 205 | Ex. 5 | 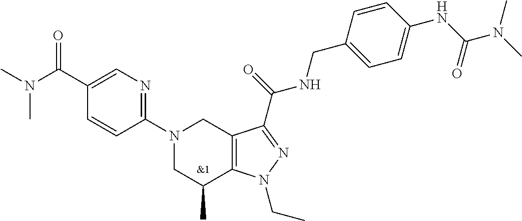 ¹H NMR* (400 MHz, CD₃OD) δ 8.31 (d, 1H), 7.74 (dd, 1H), 7.37-7.31 (m, 2H), 7.30- 7.24 (m, 2H), 6.93 (d, 1H), 5.38 (d, 1H), 4.62-4.57 (m, 3H), 4.50 (s, 2H), 3.40 (dd, 1H), 3.10 (s, 6H), 3.02-2.98 (m, 6H), 2.83- 2.67 (m, 2H), 1.41-1.33 (m, 6H). | rac-(R)-7-(5- (dimethylcarbamoyl) pyridin-2-yl)-N-(4-(3,3- dimethylureido)benzyl)- 3-ethyl-5-methyl-5,6,7,8- tetrahydroimidazo[1,5- a]pyrazine-1- carboxamide | 533.2 |
| 206 | Ex. 193 | 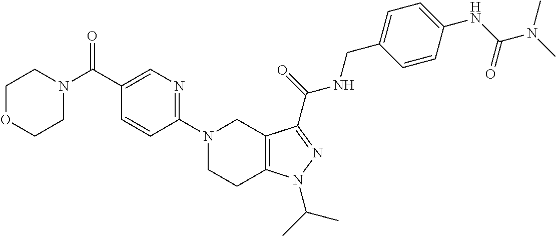 [SMILES: CC(C)n1nc(C(=O)NCc2ccc(NC(=O)N(C)C)cc2)c2c1CCN(c1ccc(C(=O)N3CCOCC3)cn1)C2] | N-(4-(3,3- dimethylureido)benzyl)- 1-isopropyl-5-(5- (morpholine-4- carbonyl)pyridin-2-yl)- 4,5,6,7-tetrahydro-1H- pyrazolo[4,3-c]pyridine- 3-carboxamide | 575.4 |
| 207 | Ex. 193 | 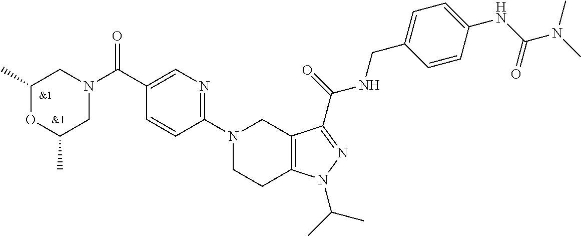 | 5-(5-((2R,6S)-2,6- dimethylmorpholine-4- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 603.5 |
| 208 | Ex. 193 | 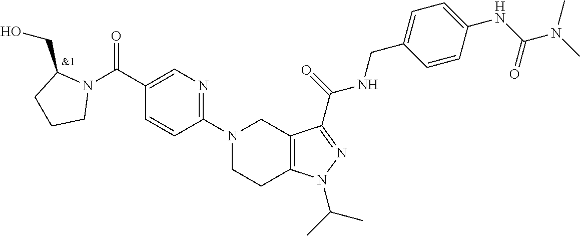 [SMILES: CC(C)n1nc(C(=O)NCc2ccc(NC(=O)N(C)C)cc2)c2c1CCN(c1ccc(C(=O)N3CCC[C@H]3CO)cn1)C2] | rac-(R)-N-(4-(3,3- dimethylureido)benzyl)- 5-(5-(2- (hydroxymethyl)pyrrolidine- 1-carbonyl)pyridin-2- yl)-1-isopropyl-4,5,6,7- tetrahydro-1H- pyrazolo[4,3-c]pyridine- 3-carboxamide | 589.5 |
| 209 | Ex. 193 | 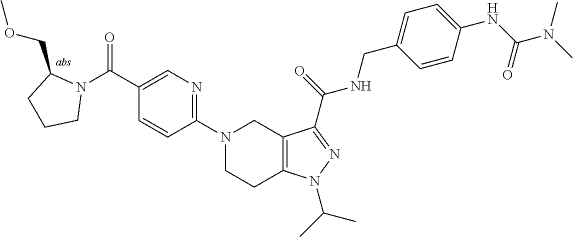 | (S)-N-(4-(3,3- dimethylureido)benzyl)- 1-isopropyl-5-(5-(2- (methoxymethyl)pyrrolidine- 1-carbonyl)pyridin-2- yl)-4,5,6,7-tetrahydro- 1H-pyrazolo[4,3- c]pyridine-3- carboxamide | 603.5 |
| 210 | Ex. 193 | 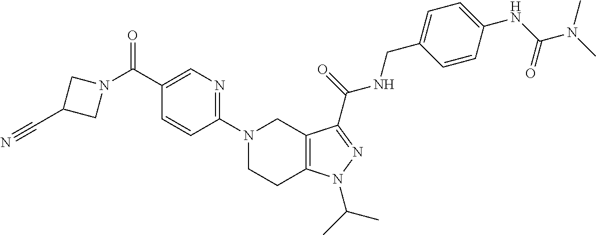 [SMILES: CC(C)n1nc(C(=O)NCc2ccc(NC(=O)N(C)C)cc2)c2c1CCN(c1ccc(C(=O)N3CC(C#N)C3)cn1)C2] | 5-(5-(3-cyanoazetidine- 1-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 570.4 |
| 211 | Ex. 193 | 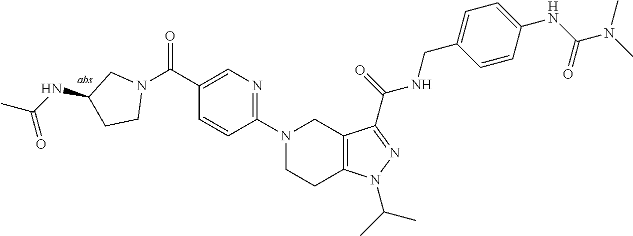 [SMILES: CC(=O)N[C@@H]1CCN(C(=O)c2ccc(N3CCc4c(c(C(=O)NCc5ccc(NC(=O)N(C)C)cc5)nn4C(C)C)C3)nc2)C1] | (R)-5-(5-(3- acetamidopyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 616.5 |
| 212 | Ex. 193 | 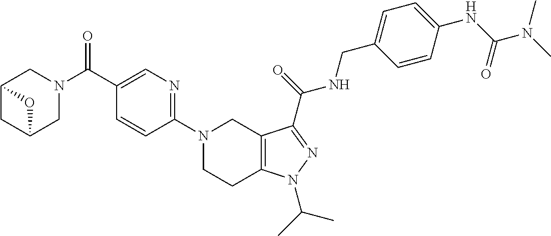 [SMILES: CC(C)n1nc(C(=O)NCc2ccc(NC(=O)N(C)C)cc2)c2c1CCN(c1ccc(C(=O)N3C[C@H]4C[C@@H](C3)O4)cn1)C2] | 5-(5-((1R,5S)-6-oxa-3- azabicyclo[3.1.1]heptane- 3-carbonyl)pyridin-2- yl)-N-(4-(3,3- dimethylureido)benzyl)- 1-isopropyl-4,5,6,7- tetrahydro-1H- pyrazolo[4,3-c]pyridine- 3-carboxamide | 587.4 |
| 213 | Ex. 193 | 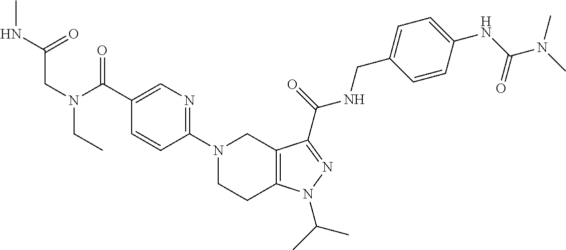 [SMILES: CCN(CC(=O)NC)C(=O)c1ccc(N2CCc3c(c(C(=O)NCc4ccc(NC(=O)N(C)C)cc4)nn3C(C)C)C2)nc1] | N-(4-(3,3- dimethylureido)benzyl)- 5-(5-(ethyl(2- (methylamino)-2- oxoethyl)carbamoyl) pyridin-2-yl)-1-isopropyl- 4,5,6,7-tetrahydro-1H- pyrazolo[4,3-c]pyridine- 3-carboxamide | 604.5 |
| 214 | Ex. 193 | 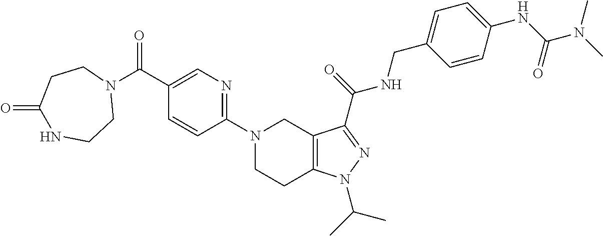 [SMILES: CC(C)n1nc(C(=O)NCc2ccc(NC(=O)N(C)C)cc2)c2c1CCN(c1ccc(C(=O)N3CCNC(=O)CC3)cn1)C2] | N-(4-(3,3- dimethylureido)benzyl)- 1-isopropyl-5-(5-(5-oxo- 1,4-diazepane-1- carbonyl)pyridin-2-yl)- 4,5,6,7-tetrahydro-1H- pyrazolo[4,3-c]pyridine- 3-carboxamide | 602.4 |
| 215 | Ex. 193 | 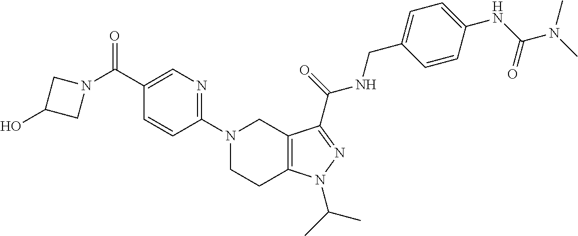 [SMILES: CC(C)n1nc(C(=O)NCc2ccc(NC(=O)N(C)C)cc2)c2c1CCN(c1ccc(C(=O)N3CC(O)C3)cn1)C2] | N-(4-(3,3- dimethylureido)benzyl)- 5-(5-(3- hydroxyazetidine-1- carbonyl)pyridin-2-yl)-1- isopropyl-4,5,6,7- tetrahydro-1H- pyrazolo[4,3-c]pyridine- 3-carboxamide | 561.4 |
| 216 | Ex. 193 | 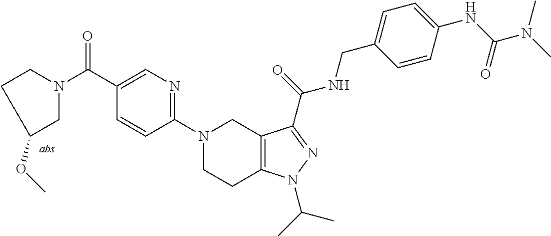 [SMILES: CO[C@@H]1CCN(C(=O)c2ccc(N3CCc4c(c(C(=O)NCc5ccc(NC(=O)N(C)C)cc5)nn4C(C)C)C3)nc2)C1] | (R)-N-(4-(3,3- dimethylureido)benzyl)- 1-isopropyl-5-(5-(3- methoxypyrrolidine-1- carbonyl)pyridin-2-yl)- 4,5,6,7-tetrahydro-1H- pyrazolo[4,3-c]pyridine- 3-carboxamide | 589.5 |
| 217 | Ex. 193 | 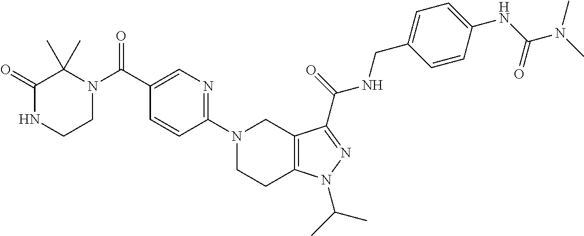 [SMILES: CC(C)n1nc(C(=O)NCc2ccc(NC(=O)N(C)C)cc2)c2c1CCN(c1ccc(C(=O)N3CCNC(=O)C3(C)C)cn1)C2] | 5-(5-(2,2-dimethyl-3- oxopiperazine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 616.4 |
| 218 | Ex. 193 | 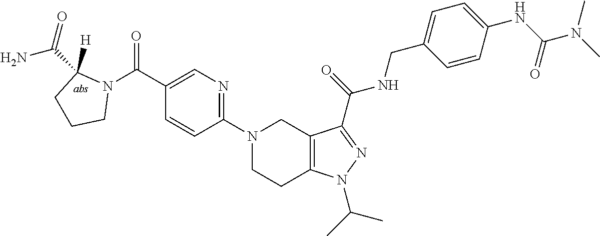 [SMILES: CC(C)n1nc(C(=O)NCc2ccc(NC(=O)N(C)C)cc2)c2c1CCN(c1ccc(C(=O)N3CCC[C@@H]3C(N)=O)cn1)C2] | (R)-5-(5-(2- carbamoylpyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 602.4 |
| 219 | Ex. 193 | 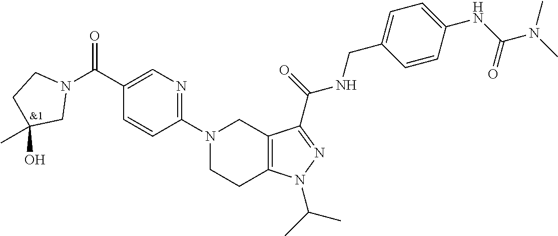 | rac-(R)-N-(4-(3,3- dimethylureido)benzyl)- 5-(5-(3-hydroxy-3- methylpyrrolidine-1- carbonyl)pyridin-2-yl)-1- isopropyl-4,5,6,7- tetrahydro-1H- pyrazolo[4,3-c]pyridine- 3-carboxamide | 589.4 |
| 220 | Ex. 193 | 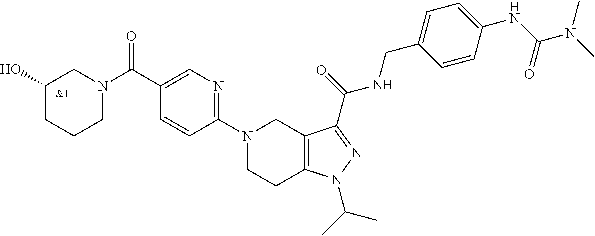 [SMILES: CC(C)n1nc(C(=O)NCc2ccc(NC(=O)N(C)C)cc2)c2c1CCN(c1ccc(C(=O)N3CCC[C@H](O)C3)cn1)C2] | rac-(R)-N-(4-(3,3- dimethylureido)benzyl)- 5-(5-(3- hydroxypiperidine-1- carbonyl)pyridin-2-yl)-1- isopropyl-4,5,6,7- tetrahydro-1H- pyrazolo[4,3-c]pyridine- 3-carboxamide | 589.4 |
| 221 | Ex. 193 | 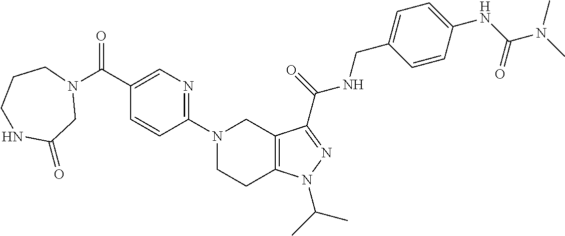 [SMILES: CC(C)n1nc(C(=O)NCc2ccc(NC(=O)N(C)C)cc2)c2c1CCN(c1ccc(C(=O)N3CCCNC(=O)C3)cn1)C2] | N-(4-(3,3- dimethylureido)benzyl)- 1-isopropyl-5-(5-(3-oxo- 1,4-diazepane-1- carbonyl)pyridin-2-yl)- 4,5,6,7-tetrahydro-1H- pyrazolo[4,3-c]pyridine- 3-carboxamide | 602.4 |
| 222 | Ex. 193 | 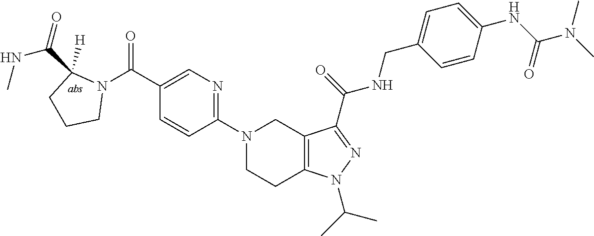 | (S)-N-(4-(3,3- dimethylureido)benzyl)- 1-isopropyl-5-(5-(2- (methylcarbamoyl)pyrrolidine- 1-carbonyl)pyridin- 2-yl)-4,5,6,7-tetrahydro- 1H-pyrazolo[4,3- c]pyridine-3- carboxamide | 616.5 |
| 223 | Ex. 193 | 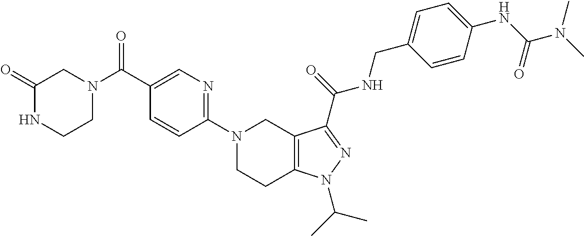 [SMILES: CC(C)n1nc(C(=O)NCc2ccc(NC(=O)N(C)C)cc2)c2c1CCN(c1ccc(C(=O)N3CCNC(=O)C3)cn1)C2] | N-(4-(3,3- dimethylureido)benzyl)- 1-isopropyl-5-(5-(3- oxopiperazine-1- carbonyl)pyridin-2-yl)- 4,5,6,7-tetrahydro-1H- pyrazolo[4,3-c]pyridine- 3-carboxamide | 588.4 |
| 224 | Ex. 193 | 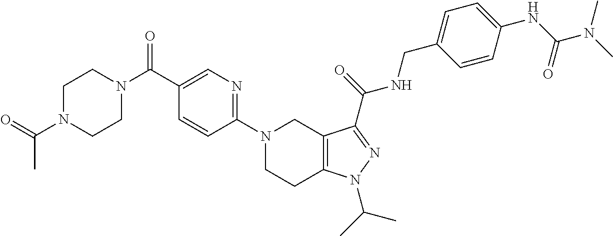 [SMILES: CC(=O)N1CCN(C(=O)c2ccc(N3CCc4c(c(C(=O)NCc5ccc(NC(=O)N(C)C)cc5)nn4C(C)C)C3)nc2)CC1] | 5-(5-(4-acetylpiperazine- 1-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 616.4 |
| 225 | Ex. 193 | 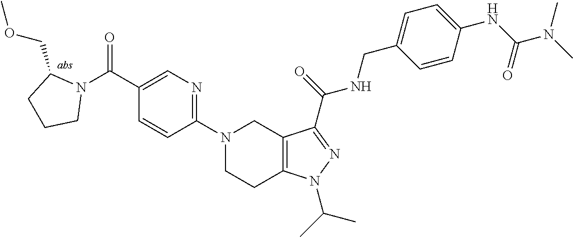 | (R)-N-(4-(3,3- dimethylureido)benzyl)- 1-isopropyl-5-(5-(2- (methoxymethyl)pyrrolidine- 1-carbonyl)pyridin-2- yl)-4,5,6,7-tetrahydro- 1H-pyrazolo[4,3- c]pyridine-3- carboxamide | 603.5 |
| 226 | Ex. 193 | 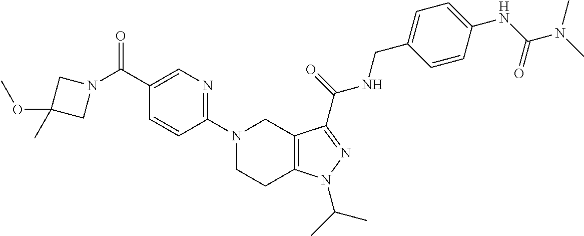 | N-(4-(3,3- dimethylureido)benzyl)- 1-isopropyl-5-(5-(3- methoxy-3- methylazetidine-1- carbonyl)pyridin-2-yl)- 4,5,6,7-tetrahydro-1H- pyrazolo[4,3-c]pyridine- 3-carboxamide | 589.4 |
| 227 | Ex. 193 | 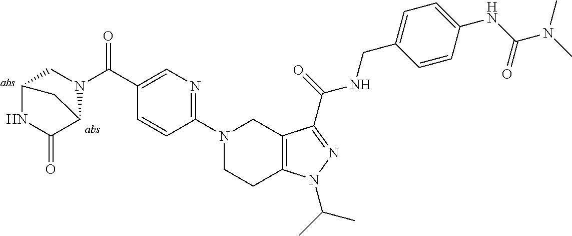 [SMILES: CC(C)n1nc(C(=O)NCc2ccc(NC(=O)N(C)C)cc2)c2c1CCN(c1ccc(C(=O)N3C[C@@H]4C[C@H]3C(=O)N4)cn1)C2] | N-(4-(3,3- dimethylureido)benzyl)- 1-isopropyl-5-(5- ((1S,4S)-6-oxo-2,5- diazabicyclo[2.2.1]heptane- 2-carbonyl)pyridin-2- yl)-4,5,6,7-tetrahydro- 1H-pyrazolo[4,3- c]pyridine-3- carboxamide | 600.7 |
| 228 | Ex. 193 | 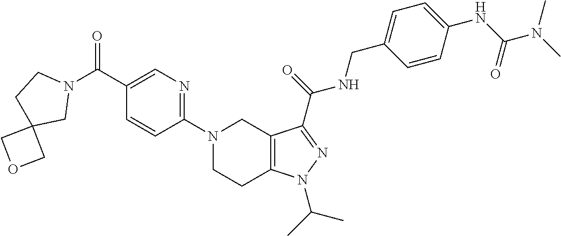 [SMILES: CC(C)n1nc(C(=O)NCc2ccc(NC(=O)N(C)C)cc2)c2c1CCN(c1ccc(C(=O)N3CCC4(COC4)C3)cn1)C2] | 5-(5-(2-oxa-6- azaspiro[3.4]octane-6- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 601.4 |
| 229 | Ex. 193 | 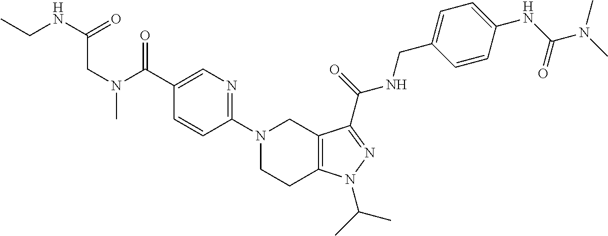 [SMILES: CCNC(=O)CN(C)C(=O)c1ccc(N2CCc3c(c(C(=O)NCc4ccc(NC(=O)N(C)C)cc4)nn3C(C)C)C2)nc1] | N-(4-(3,3- dimethylureido)benzyl)- 5-(5-((2-(ethylamino)-2- oxoethyl)(methyl)carbamoyl) pyridin-2-yl)-1- isopropyl-4,5,6,7- tetrahydro-1H- pyrazolo[4,3-c]pyridine- 3-carboxamide | 604.5 |
| 230^{∧} | Ex. 128 | 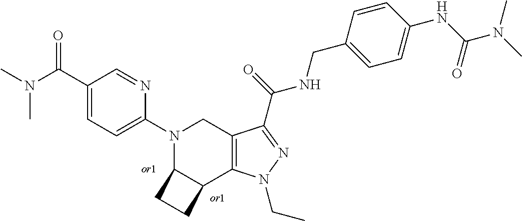 ¹H NMR (600 MHz, (CD₃)₂SO) δ 8.51 (t, 1H), 8.28 (d, 1H), 8.24 (s, 1H), 7.68 (dd, 1H), 7.42-7.37 (m, 2H), 7.22-7.18 (m, 2H), 6.89 (d, 1H), 5.53 (q, 1H), 5.33 (d, 1H), 4.42-4.30 (m, 2H), 4.24 (d, 1H), 4.12- 3.99 (m, 2H), 3.77 (t, 1H), 2.99 (s, 6H), 2.92 (s, 6H), 2.32-2.22 (m, 1H), 2.21-2.07 (m, 2H), 1.65 (t, 1H), 1.38 (t, 3H). | rel-(5aR,7aS)-5-(5- (dimethylcarbamoyl) pyridin-2-yl)-N-(4-(3,3- dimethylureido)benzyl)- 1-ethyl-4,5,5a,6,7,7a- hexahydro-1H- cyclobuta[b]pyrazolo[3,4- d]pyridine-3- carboxamide | 545.2 |
| 231^{∧} | Ex. 128 | 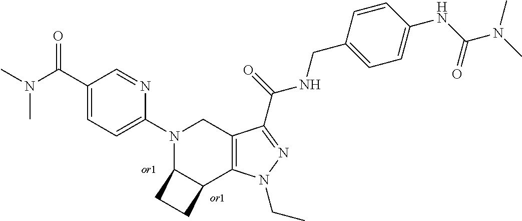 ¹H NMR (600 MHz, (CD₃)₂SO) δ 8.51 (t, 1H), 8.28 (d, 1H), 8.24 (s, 1H), 7.68 (dd, 1H), 7.42-7.37 (m, 2H), 7.21-7.18 (m, 2H), 6.88 (d, 1H), 5.52 (q, 1H), 5.33 (d, 1H), 4.41-4.30 (m, 2H), 4.24 (d, 1H), 4.12- 3.99 (m, 2H), 3.77 (t, 1H), 2.98 (s, 6H), 2.92 (s, 6H), 2.30-2.24 (m, 1H), 2.22-2.06 (m, 2H), 1.65 (t, 1H), 1.38 (t, 3H). | rel-(5aR,7aS)-5-(5- (dimethylcarbamoyl) pyridin-2-yl)-N-(4-(3,3- dimethylureido)benzyl)- 1-ethyl-4,5,5a,6,7,7a- hexahydro-1H- cyclobuta[b]pyrazolo[3,4- d]pyridine-3- carboxamide | 545.3 |
| 232^{∧} | Ex. 5 | 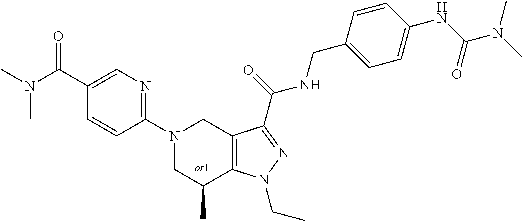 ¹H NMR (400 MHz, CD₃OD) δ 8.32 (d, 1H), 7.75 (dd, 1H), 7.39-7.32 (m, 2H), 7.30- 7.24 (m, 2H), 6.94 (d, 1H), 5.38 (d, 1H), 4.86-4.84 (m, 1H), 4.65-4.58 (m, 2H), 4.50 (s, 2H), 3.40 (dd, 1H), 3.11 (s, 6H), 3.01 (s, 6H), 2.86-2.66 (m, 2H), 1.43- 1.33 (m, 6H). | rel-(R or S)-7-(5- (dimethylcarbamoyl) pyridin-2-yl)-N-(4-(3,3- dimethylureido)benzyl)- 3-ethyl-5-methyl-5,6,7,8- tetrahydroimidazo[1,5- a]pyrazine-1- carboxamide | 533.3 |
| 233^{∧} | Ex. 5 | 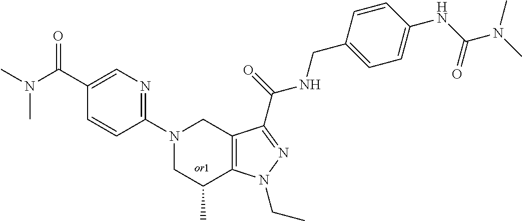 ¹H NMR (400 MHz, CD₃OD) δ 8.32 (d, 1H), 7.74 (dd, 1H), 7.39-7.32 (m, 2H), 7.31- 7.25 (m, 2H), 6.93 (d, 1H), 5.38 (d, 1H), 4.86-4.84 (m, 1H), 4.66-4.55 (m, 2H), 4.50 (s, 2H), 3.44-3.36 (m, 1H), 3.11 (s, 6H), 3.01 (s, 6H), 2.81-2.67 (m, 2H), 1.42- 1.32 (m, 6H). | rel-(R or S)-7-(5- (dimethylcarbamoyl) pyridin-2-yl)-N-(4-(3,3- dimethylureido)benzyl)- 3-ethyl-5-methyl-5,6,7,8- tetrahydroimidazo[1,5- a]pyrazine-1- carboxamide | 533.3 |
| 234^{∧} | Ex. 43 | 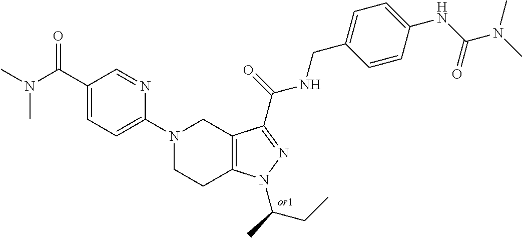 ¹H NMR (400 MHz, (CD₃)₂SO) δ 8.32 (t, 1H), 8.27-8.20 (m, 2H), 7.64 (dd, 1H), 7.41-7.36 (m, 2H), 7.21-7.14 (m, 2H), 6.88 (d, 1H), 4.74 (s, 2H), 4.38-4.31 (m, 2H), 4.27-4.16 (m, 1H), 4.01-3.87 (m, 2H), 2.97 (s, 6H), 2.90 (s, 6H), 2.84-2.76 (m, 2H), 1.89-1.64 (m, 2H), 1.39 (d, 3H), 0.69 (t, 3H). | rel-(R or S)-1-(sec- butyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | |
| 235^{∧} | Ex. 43 | 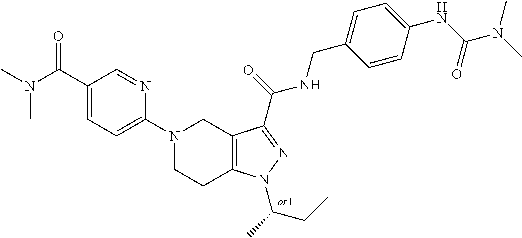 ¹H NMR (400 MHz, (CD₃)₂SO) δ 8.35- 8.28 (m, 1H), 8.26-8.20 (m, 2H), 7.64 (dd, 1H), 7.41-7.37 (m, 2H), 7.21-7.14 (m, 2H), 6.88 (d, 1H), 4.79-4.68 (m, 2H), 4.40- 4.29 (m, 2H), 4.27-4.17 (m, 1H), 4.02- 3.88 (m, 2H), 3.81-3.73 (m, 1H), 2.97 (s, 6H), 2.91 (s, 6H), 2.83-2.75 (m, 2H), 1.92- 1.65 (m, 1H), 1.41-1.35 (m, 3H), 0.70 (t, 3H). | rel-(R or S)-1-(sec- butyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | |
| 236 | Ex. 236 | 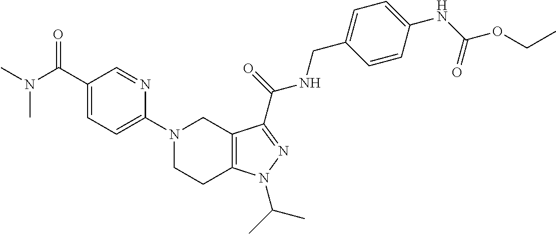 [SMILES: CCOC(=O)Nc1ccc(CNC(=O)c2nn(C(C)C)c3c2CN(c2ccc(C(=O)N(C)C)cn2)CC3)cc1] ¹H NMR (400 MHz, (CD₃)₂SO) δ 9.46 (s, 1H), 8.28 (t, 1H), 8.16-8.14 (m, 1H), 7.55 (dd, 1H), 7.30 (d, 2H), 7.14 (d, 2H), 6.80 (d, 1H), 4.66 (s, 2H), 4.46-4.36 (m, 1H), 4.27 (d, 2H), 4.02 (q, 2H), 3.86 (t, 2H), 2.88 (s, 6H), 2.73 (t, 2H), 1.31 (d, 6H), 1.14 (t, 3H). | ethyl (4-((5-(5- (dimethylcarbamoyl) pyridin-2-yl)-1-isopropyl- 4,5,6,7-tetrahydro-1H- pyrazolo[4,3-c]pyridine- 3- carboxamido)methyl) phenyl)carbamate | 534.3 |
| 237^{∧} | Ex. 5 | 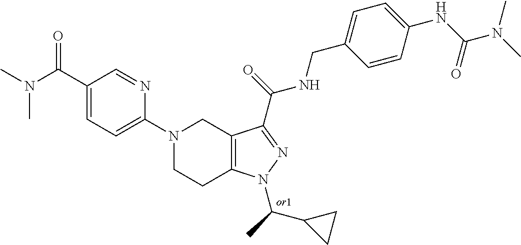 ¹H NMR (400 MHz, CD₃OD) δ 8.28 (d, 1H), 7.69 (dd, 1H), 7.37-7.33 (m, 2H), 7.32- 7.27 (m, 2H), 6.91 (d, 1H), 4.84-4.70 (m, 2H), 4.52 (s, 2H), 4.20-4.09 (m, 1H), 4.06- 3.96 (m, 1H), 3.71-3.60 (m, 1H), 3.10 (s, 6H), 3.02 (s, 6H), 2.84 (t, 2H), 1.58 (d, 3H), 1.46-1.36 (m, 1H), 0.71-0.60 (m, 1H), 0.51-0.39 (m, 1H), 0.38-0.24 (m, 2H). | rel-(R or S)-1-(1- cyclopropylethyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 559.3 |
| 238 | Ex. 128 | 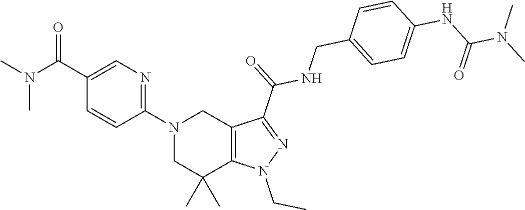 ¹H NMR* (600 MHz, (CD₃)₂SO) δ 8.43 (t, 1H), 8.24-8.21 (m, 2H), 7.64 (dd, 1H), 7.40-7.36 (m, 2H), 7.18-7.14 (m, 2H), 6.92 (d, 1H), 4.67 (s, 2H), 4.33 (d, 2H), 4.23- 4.17 (m, 2H), 3.68 (s, 2H), 3.01-2.93 (m, 6H), 2.90 (s, 6H), 1.41 (t, 3H), 1.34 (s, 6H). | 5-(5- (dimethylcarbamoyl) pyridin-2-yl)-N-(4-(3,3- dimethylureido)benzyl)- 1-ethyl-7,7-dimethyl- 4,5,6,7-tetrahydro-1H- pyrazolo[4,3-c]pyridine- 3-carboxamide | 547.5 |
| 239 | Ex. 193 | 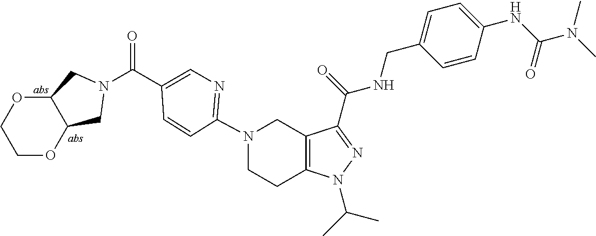 [SMILES: CC(C)n1nc(C(=O)NCc2ccc(NC(=O)N(C)C)cc2)c2c1CCN(c1ccc(C(=O)N3C[C@@H]4OCCO[C@@H]4C3)cn1)C2] | N-(4-(3,3- dimethylureido)benzyl)- 5-(5-((4aR,7aS)- hexahydro-5H- [1,4]dioxino[2,3- c]pyrrole-6- carbonyl)pyridin-2-yl)-1- isopropyl-4,5,6,7- tetrahydro-1H- pyrazolo[4,3-c]pyridine- 3-carboxamide | 617.8 |
| 240 | Ex. 193 | 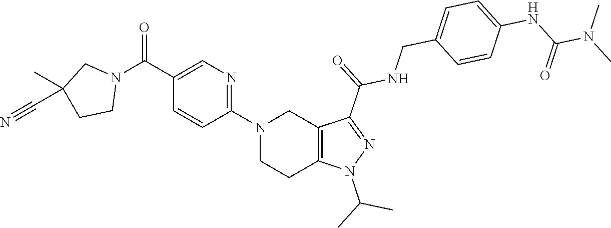 [SMILES: CC(C)n1nc(C(=O)NCc2ccc(NC(=O)N(C)C)cc2)c2c1CCN(c1ccc(C(=O)N3CCC(C)(C#N)C3)cn1)C2] | 5-(5-(3-cyano-3- methylpyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 598.8 |
| 241 | Ex. 193 | 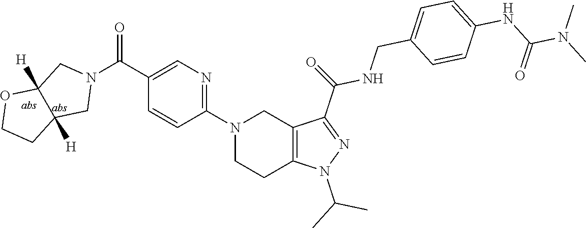 [SMILES: CC(C)n1nc(C(=O)NCc2ccc(NC(=O)N(C)C)cc2)c2c1CCN(c1ccc(C(=O)N3C[C@@H]4CCO[C@@H]4C3)cn1)C2] | N-(4-(3,3- dimethylureido)benzyl)- 5-(5-((3aS,6aS)- hexahydro-2H-furo[2,3- c]pyrrole-5- carbonyl)pyridin-2-yl)-1- isopropyl-4,5,6,7- tetrahydro-1H- pyrazolo[4,3-c]pyridine- 3-carboxamide | 601.7 |
| 242 | Ex. 193 | 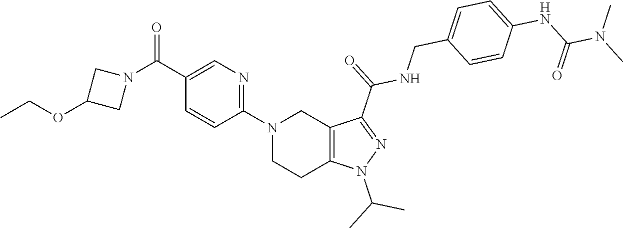 [SMILES: CCOC1CN(C(=O)c2ccc(N3CCc4c(c(C(=O)NCc5ccc(NC(=O)N(C)C)cc5)nn4C(C)C)C3)nc2)C1] | N-(4-(3,3- dimethylureido)benzyl)- 5-(5-(3-ethoxyazetidine- 1-carbonyl)pyridin-2-yl)- 1-isopropyl-4,5,6,7- tetrahydro-1H- pyrazolo[4,3-c]pyridine- 3-carboxamide | 589.8 |
| 243 | Ex. 193 | 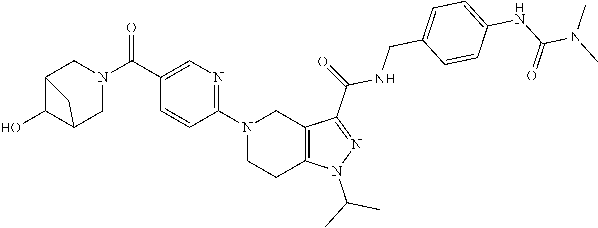 [SMILES: CC(C)n1nc(C(=O)NCc2ccc(NC(=O)N(C)C)cc2)c2c1CCN(c1ccc(C(=O)N3CC4CC(C3)C4O)cn1)C2] | N-(4-(3,3- dimethylureido)benzyl)- 5-(5-(6-hydroxy-3- azabicyclo[3.1.1]heptane- 3-carbonyl)pyridin-2- yl)-1-isopropyl-4,5,6,7- tetrahydro-1H- pyrazolo[4,3-c]pyridine- 3-carboxamide | 601.7 |
| 244 | Ex. 193 | 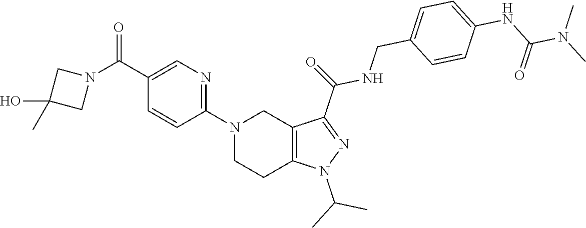 [SMILES: CC(C)n1nc(C(=O)NCc2ccc(NC(=O)N(C)C)cc2)c2c1CCN(c1ccc(C(=O)N3CC(C)(O)C3)cn1)C2] | N-(4-(3,3- dimethylureido)benzyl)- 5-(5-(3-hydroxy-3- methylazetidine-1- carbonyl)pyridin-2-yl)-1- isopropyl-4,5,6,7- tetrahydro-1H- pyrazolo[4,3-c]pyridine- 3-carboxamide | 575.8 |
| 245 | Ex. 193 | 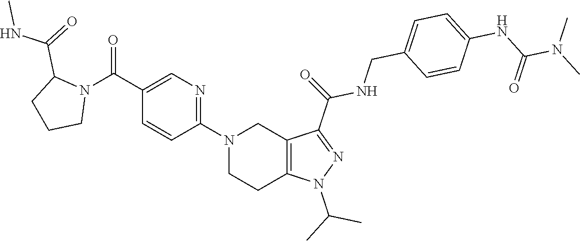 | rac-(R)-N-(4-(3,3- dimethylureido)benzyl)- 1-isopropyl-5-(5-(2- (methylcarbamoyl)pyrrolidine- 1-carbonyl)pyridin- 2-yl)-4,5,6,7-tetrahydro- 1H-pyrazolo[4,3- c]pyridine-3- carboxamide | 616.8 |
| 246 | Ex. 2^{bd} | 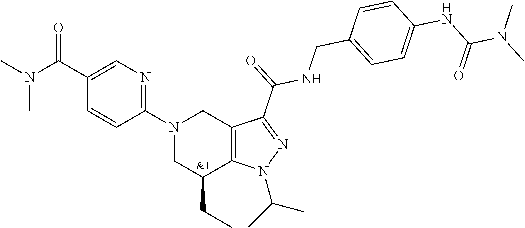 ¹H NMR* (600 MHz, (CD₃)₂SO) δ 8.36 (t, 1H), 8.26-8.20 (m, 2H), 7.67 (dd, 1H), 7.41-7.36 (m, 2H), 7.21-7.14 (m, 2H), 6.85 (d, 1H), 5.04-4.98 (m, 1H), 4.81 (d, 1H), 4.56-4.48 (m, 1H), 4.39-4.29 (m, 2H), 4.21 (d, 1H), 3.19-3.12 (m, 1H), 3.04- 2.94 (m, 6H), 2.92-2.89 (m, 6H), 1.57- 1.51 (m, 1H), 1.48-1.37 (m, 8H), 1.06- 1.00 (m, 3H). | rac-(R)-5-(5- (dimethylcarbamoyl) pyridin-2-yl)-N-(4-(3,3- dimethylureido)benzyl)- 7-ethyl-1-isopropyl- 4,5,6,7-tetrahydro-1H- pyrazolo[4,3-c]pyridine- 3-carboxamide | 561.5 |
| 247 | Ex. 125 | 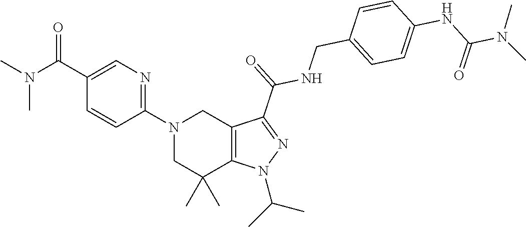 ¹H NMR (600 MHz, (CD₃)₂SO) δ 8.37 (t, 1H), 8.24-8.19 (m, 2H), 7.67 (dd, 1H), 7.41-7.37 (m, 2H), 7.19-7.14 (m, 2H), 6.98 (d, 1H), 4.74-4.67 (m, 3H), 4.35 (d, 2H), 3.69 (s, 2H), 2.97 (s, 6H), 2.90 (s, 6H), 1.43 (d, 6H), 1.35 (s, 6H). | 5-(5- (dimethylcarbamoyl) pyridin-2-yl)-N-(4-(3,3- dimethylureido)benzyl)- 1-isopropyl-7,7- dimethyl-4,5,6,7- tetrahydro-1H- pyrazolo[4,3-c]pyridine- 3-carboxamide | 561.5 |
| 248 | Ex. 113 | 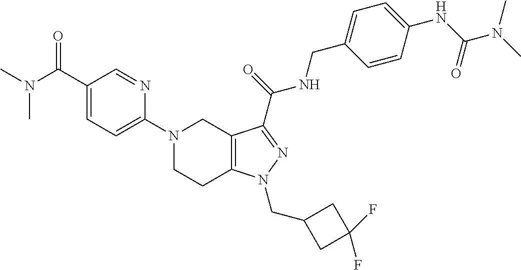 ¹H NMR (400 MHz, (CD₃)₂SO) δ 8.46 (t, 1H), 8.25-8.20 (m, 2H), 7.63 (dd, 1H), 7.40-7.35 (m, 2H), 7.18-7.13 (m, 2H), 6.88 (d, 1H), 4.74 (s, 2H), 4.33 (d, 2H), 4.16 (d, 2H), 3.94 (t, 2H), 2.96 (s, 6H), 2.90 (s, 6H), 2.80 (t, 2H), 2.69-2.58 (m, 3H), 2.47- 2.38 (m, 2H). | 1-((3,3- difluorocyclobutyl)methyl)- 5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 595.3 |
| 249 | Ex. 128^{de} | 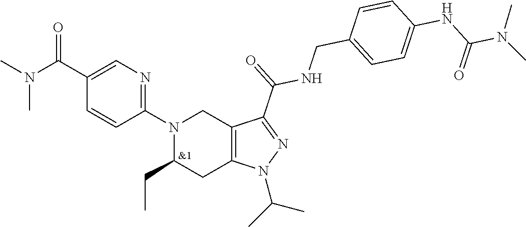 ¹H NMR* (600 MHz, (CD₃)₂SO) δ 8.34 (t, 1H), 8.26-8.19 (m, 2H), 7.65 (dd, 1H), 7.43-7.37 (m, 2H), 7.21-7.14 (m, 2H), 6.89 (d, 1H), 5.21 (d, 1H), 5.02 (s, 1H), 4.56- 4.48 (m, 1H), 4.40-4.29 (m, 2H), 4.09 (d, 2H), 3.00-2.94 (m, 6H), 2.94-2.87 (m, 7H), 2.83 (d, 1H), 1.54-1.40 (m, 4H), 1.37 (d, 3H), 0.79 (t, 3H). | rac-(R)-5-(5- (dimethylcarbamoyl) pyridin-2-yl)-N-(4-(3,3- dimethylureido)benzyl)- 6-ethyl-1-isopropyl- 4,5,6,7-tetrahydro-1H- pyrazolo[4,3-c]pyridine- 3-carboxamide | 561.2 |
| 250^{∧} | Ex. 113^{f} | 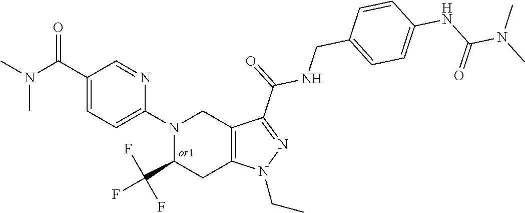 ¹H NMR (600 MHz, (CD₃)₂SO) δ 8.54 (t, 1H), 8.33 (d, 1H), 8.22 (s, 1H), 7.78 (dd, 1H), 7.40-7.36 (m, 2H), 7.20-7.16 (m, 2H), 7.02 (d, 1H), 6.31-6.21 (m, 1H), 5.05 (d, 1H), 4.38-4.06 (m, 5H), 3.30-3.14 (m, 2H), 2.98 (s, 6H), 2.90 (s, 6H), 1.33 (t, 3H). | rel-(R or S)-5-(5- (dimethylcarbamoyl) pyridin-2-yl)-N-(4-(3,3- dimethylureido)benzyl)- 1-ethyl-6- (trifluoromethyl)-4,5,6,7- tetrahydro-1H- pyrazolo[4,3-c]pyridine- 3-carboxamide | 587.2 |
| 251^{∧} | Ex. 113^{f} | 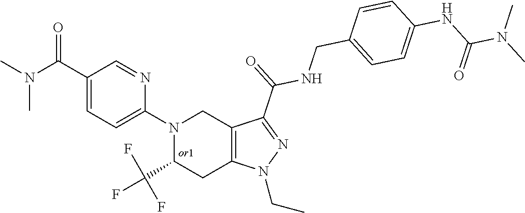 ¹H NMR (600 MHz, (CD₃)₂SO) δ 8.54 (t, 1H), 8.33 (d, 1H), 8.22 (s, 1H), 7.78 (dd, 1H), 7.41-7.36 (m, 2H), 7.20-7.15 (m, 2H), 7.02 (d, 1H), 6.31-6.23 (m, 1H), 5.05 (d, 1H), 4.38-4.08 (m, 5H), 3.30-3.14 (m, 2H), 2.98 (s, 6H), 2.90 (s, 6H), 1.33 (t, 3H). | rel-(R or S)-5-(5- (dimethylcarbamoyl) pyridin-2-yl)-N-(4-(3,3- dimethylureido)benzyl)- 1-ethyl-6- (trifluoromethyl)-4,5,6,7- tetrahydro-1H- pyrazolo[4,3-c]pyridine- 3-carboxamide | 587.2 |
| 252 | Ex. 252 | 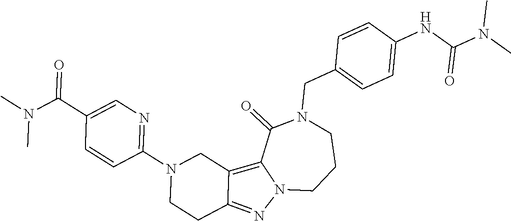 ¹H NMR* (600 MHz, (CD₃)₂SO) δ 8.28 (s, 1H), 8.24 (d, 1H), 7.64 (dd, 1H), 7.48-7.44 (m, 2H), 7.27-7.23 (m, 2H), 6.92 (d, 1H), 4.74 (s, 2H), 4.60 (s, 2H), 4.24 (t, 2H), 3.94 (t, 2H), 3.33 (t, 2H), 2.97 (s, 6H), 2.92 (s, 6H), 2.73 (t, 2H), 1.99-1.92 (m, 2H). | 6-(10-(4-(3,3- dimethylureido)benzyl)- 11-oxo- 1,3,4,7,8,9,10,11- octahydro-2H- pyrido[4′,3′:3,4]pyrazolo [1,5-a][1,4]diazepin-2-yl)- N,N- dimethylnicotinamide | 531.4 |
| 253 | Ex. 113 | 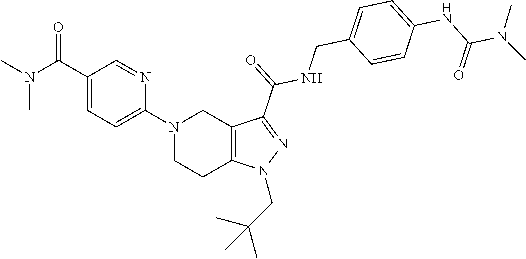 ¹H NMR (400 MHz, (CD₃)₂SO) δ 8.32 (t, 1H), 8.26-8.21 (m, 2H), 7.64 (dd, 1H), 7.41-7.36 (m, 2H), 7.20-7.14 (m, 2H), 6.89 (d, 1H), 4.74 (s, 2H), 4.35 (d, 2H), 3.94 (t, 2H), 3.83 (s, 2H), 2.97 (s, 6H), 2.91 (s, 6H), 2.80 (t, 2H), 0.93 (s, 9H). | 5-(5- (dimethylcarbamoyl) pyridin-2-yl)-N-(4-(3,3- dimethylureido)benzyl)- 1-neopentyl-4,5,6,7- tetrahydro-1H- pyrazolo[4,3-c]pyridine- 3-carboxamide | 561.3 |
| 254 | Ex. 113 | 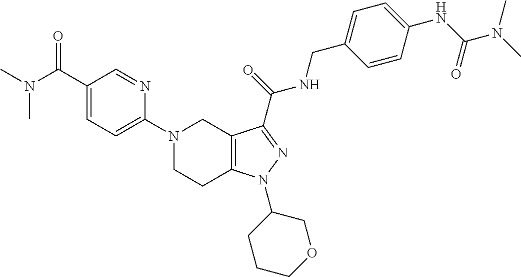 ¹H NMR (400 MHz, (CD₃)₂SO) δ 8.37 (t, 1H), 8.23-8.21 (m, 2H), 7.62 (dd, 1H), 7.40-7.34 (m, 2H), 7.16 (d, 2H), 6.87 (d, 1H), 4.73 (s, 2H), 4.33 (d, 2H), 4.28-4.15 (m, 1H), 3.98-3.80 (m, 4H), 3.54 (t, 1H), 3.41-3.34 (m, 1H), 2.96 (s, 6H), 2.89 (s, 6H), 2.85-2.80 (m, 2H), 2.17-1.99 (m, 2H), 1.80-1.61 (m, 2H). | 5-(5- (dimethylcarbamoyl) pyridin-2-yl)-N-(4-(3,3- dimethylureido)benzyl)- 1-(tetrahydro-2H-pyran- 3-yl)-4,5,6,7-tetrahydro- 1H-pyrazolo[4,3- c]pyridine-3- carboxamide | 575.4 |
| 255 | Ex. 113^{df} | 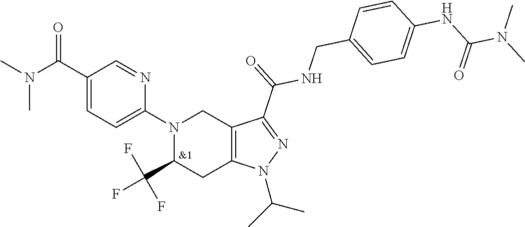 ¹H NMR* (600 MHz, (CD₃)₂SO) δ 8.42 (t, 1H), 8.33 (d, 1H), 8.23 (s, 1H), 7.78 (dd, 1H), 7.42-7.37 (m, 2H), 7.22-7.14 (m, 2H), 7.03-6.98 (m, 1H), 6.32-6.22 (m, 1H), 5.04 (d, 1H), 4.68-4.57 (m, 1H), 4.40- 4.23 (m, 3H), 3.31-3.19 (m, 2H), 2.98 (s, 6H), 2.91 (s, 6H), 1.41 (dd, 6H). | rac-(R)-5-(5- (dimethylcarbamoyl) pyridin-2-yl)-N-(4-(3,3- dimethylureido)benzyl)- 1-isopropyl-6- (trifluoromethyl)-4,5,6,7- tetrahydro-1H- pyrazolo[4,3-c]pyridine- 3-carboxamide | 601.2 |
| 256 | Ex. 252 | 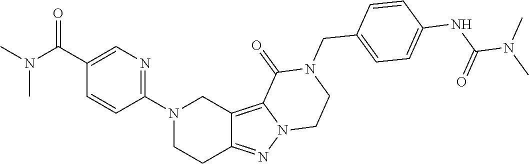 | 6-(9-(4-(3,3- dimethylureido)benzyl)- 10-oxo-3,4,7,8,9,10- hexahydropyrido[4′,3′:3, 4]pyrazolo[1,5- a]pyrazin-2(1H)-yl)-N,N- dimethylnicotinamide | 517.4 |
| 257 | Ex. 113 | 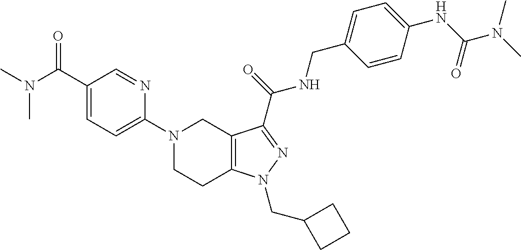 ¹H NMR (400 MHz, (CD₃)₂SO) δ 8.40 (t, 1H), 8.25-8.21 (m, 2H), 7.64 (dd, 1H), 7.41-7.36 (m, 2H), 7.19-7.14 (m, 2H), 6.88 (d, 1H), 4.74 (s, 2H), 4.33 (d, 2H), 4.07 (d, 2H), 3.97-3.91 (m, 2H), 2.97 (s, 6H), 2.91 (s, 6H), 2.84-2.68 (m, 3H), 2.00- 1.90 (m, 2H), 1.87-1.69 (m, 4H). | 1-(cyclobutylmethyl)-5- (5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 559.4 |
| 258^{∧} | Ex. 113 | 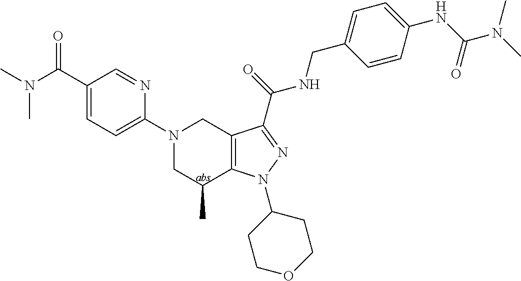 ¹H NMR (400 MHz, (CD₃)₂SO) δ 8.43 (t, 1H), 8.26-8.20 (m, 2H), 7.63 (dd, 1H), 7.41-7.36 (m, 2H), 7.17 (d, 2H), 6.89 (d, 1H), 5.09 (d, 1H), 4.50-4.17 (m, 5H), 4.01- 3.91 (m, 2H), 3.56-3.46 (m, 2H), 3.31- 3.19 (m, 2H), 2.97 (s, 6H), 2.90 (s, 6H), 2.23-2.02 (m, 2H), 1.86-1.73 (m, 2H), 1.24-1.17 (m, 3H). | (S)-5-(5- (dimethylcarbamoyl) pyridin-2-yl)-N-(4-(3,3- dimethylureido)benzyl)- 7-methyl-1-(tetrahydro- 2H-pyran-4-yl)-4,5,6,7- tetrahydro-1H- pyrazolo[4,3-c]pyridine- 3-carboxamide | 589.3 |
| 259 | Ex. 42 | 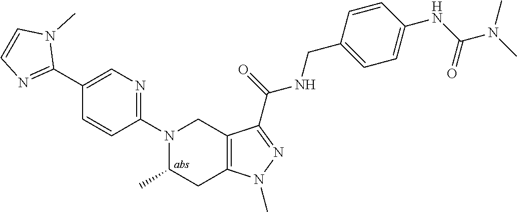 [SMILES: C[C@H]1Cc2c(c(C(=O)NCc3ccc(NC(=O)N(C)C)cc3)nn2C)CN1c1ccc(-c2nccn2C)cn1] ¹H NMR* (400 MHz, CD₃OD) δ 8.49-8.36 (m, 2H), 7.83 (dd, 1H), 7.37-7.32 (m, 2H), 7.31-7.26 (m, 3H), 7.18 (d, 1H), 6.98 (d, 1H), 5.56-5.45 (m, 1H), 5.11 (d, 1H), 4.55- 4.48 (m, 2H), 4.28 (d, 1H), 3.84-3.78 (m, 6H), 3.07-2.99 (m, 7H), 2.75 (d, 1H), 1.17 (d, 3H). | (S)-N-(4-(3,3- dimethylureido)benzyl)- 1,6-dimethyl-5-(5-(1- methyl-1H-imidazol-2- yl)pyridin-2-yl)-4,5,6,7- tetrahydro-1H- pyrazolo[4,3-c]pyridine- 3-carboxamide | 528.3 |
| 260 | Ex. 113 | 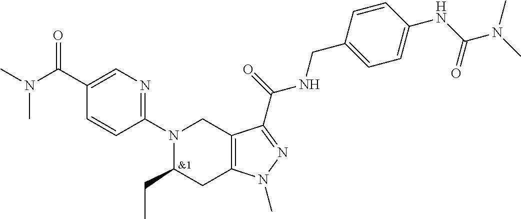 ¹H NMR (400 MHz, (CD₃)₂SO) δ 8.49 (t, 1H), 8.26-8.20 (m, 2H), 7.63 (dd, 1H), 7.41-7.35 (m, 2H), 7.19-7.13 (m, 2H), 6.87 (d, 1H), 5.30-5.20 (m, 1H), 5.06- 4.95 (m, 1H), 4.39-4.25 (m, 2H), 4.05 (d, 1H), 3.80-3.70 (m, 3H), 2.98 (s, 6H), 2.93- 2.85 (m, 7H), 2.82-2.72 (m, 1H), 1.58- 1.39 (m, 2H), 0.81 (t, 3H). | rac-(R)-5-(5- (dimethylcarbamoyl) pyridin-2-yl)-N-(4-(3,3- dimethylureido)benzyl)- 6-ethyl-1-methyl-4,5,6,7- tetrahydro-1H- pyrazolo[4,3-c]pyridine- 3-carboxamide | 533.2 |
| 261 | Ex. 9 | 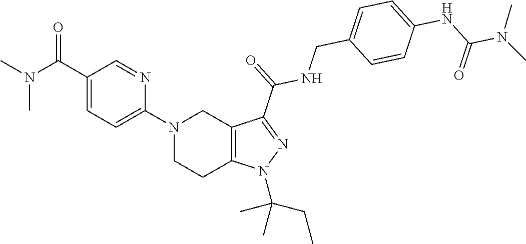 ¹H NMR (400 MHz, (CD₃)₂SO) δ 8.29 (t, 1H), 8.22 (s, 1H), 8.16 (d, 1H), 7.42-7.35 (m, 2H), 7.17 (d, 2H), 6.75 (s, 1H), 6.56 (d, 1H), 4.67 (s, 2H), 4.34 (d, 2H), 3.87 (t, 2H), 3.00-2.93 (m, 6H), 2.90 (s, 6H), 2.85 (s, 2H), 1.87 (q, 2H), 1.55 (s, 6H), 0.64 (t, 3H). | 5-(5- (dimethylcarbamoyl) pyridin-2-yl)-N-(4-(3,3- dimethylureido)benzyl)- 1-(tert-pentyl)-4,5,6,7- tetrahydro-1H- pyrazolo[4,3-c]pyridine- 3-carboxamide | 561.3 |
| 262^{∧} | Ex. 113 | 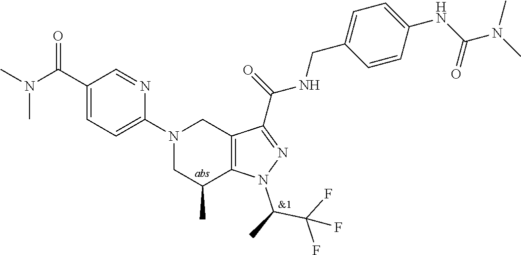 ¹H NMR (400 MHz, CDCl₃) δ 8.32 (d, 1H), 7.67-7.62 (m, 1H), 7.39-7.34 (m, 2H), 7.31-7.28 (m, 2H), 7.14 (t, 1H), 6.78 (d, 1H), 6.31 (s, 1H), 5.01 (d, 1H), 4.92-4.85 (m, 1H), 4.70-4.57 (m, 2H), 4.53-4.37 (m, 2H), 3.36-3.28 (m, 1H), 3.16-3.07 (m, 8H), 3.05-3.01 (m, 6H), 1.78 (d, 3H), 1.26 (d, 3H). | (S)-5-(5- (dimethylcarbamoyl) pyridin-2-yl)-N-(4-(3,3- dimethylureido)benzyl)- 7-methyl-1-((RS)-1,1,1- trifluoropropan-2-yl)- 4,5,6,7-tetrahydro-1H- pyrazolo[4,3-c]pyridine- 3-carboxamide | 601.3 |
| 263 | Ex. 113 | 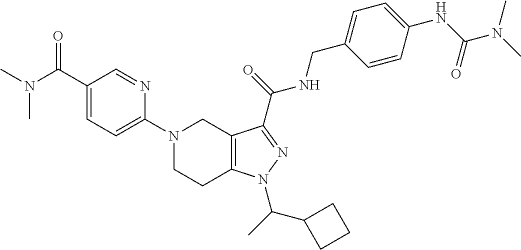 ¹H NMR (400 MHz, (CD₃)₂SO) δ 8.31 (t, 1H), 8.26-8.19 (m, 2H), 7.64 (dd, 1H), 7.43-7.34 (m, 2H), 7.18 (d, 2H), 6.88 (d, 1H), 4.79-4.60 (m, 2H), 4.39-4.20 (m, 3H), 4.04-3.86 (m, 2H), 2.97 (s, 6H), 2.91 (s, 6H), 2.86-2.73 (m, 3H), 2.08-1.96 (m, 1H), 1.82-1.56 (m, 5H), 1.29 (d, 3H). | 1-(1-cyclobutylethyl)-5- (5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 573.4 |
| 264 | Ex. 113 | 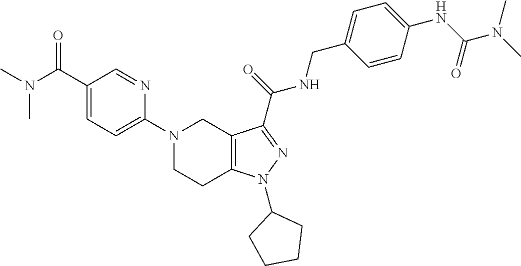 ¹H NMR (400 MHz, (CD₃)₂SO) δ 8.33 (t, 1H), 8.25-8.20 (m, 2H), 7.64 (dd, 1H), 7.42-7.36 (m, 2H), 7.20-7.14 (m, 2H), 6.89 (d, 1H), 4.75 (s, 2H), 4.71-4.57 (m, 1H), 4.35 (d, 2H), 3.95 (t, 2H), 2.97 (s, 6H), 2.91 (s, 6H), 2.85-2.78 (m, 2H), 2.09- 1.90 (m, 4H), 1.87-1.78 (m, 2H), 1.66- 1.56 (m, 2H). | 1-cyclopentyl-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 559.3 |
| 265 | Ex. 113 | 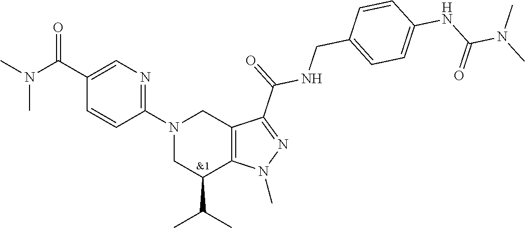 ¹H NMR (400 MHz, (CD₃)₂SO) δ 8.57- 8.48 (m, 1H), 8.32-8.18 (m, 2H), 7.71- 7.63 (m, 1H), 7.44-7.34 (m, 2H), 7.22- 7.12 (m, 2H), 6.83-6.74 (m, 1H), 5.02- 4.78 (m, 2H), 4.42-4.24 (m, 3H), 3.86- 3.79 (m, 3H), 3.12-2.95 (m, 8H), 2.94- 2.87 (m, 6H), 1.96-1.83 (m, 1H), 1.05- 0.95 (m, 3H), 0.91-0.82 (m, 3H). | rac-(R)-5-(5- (dimethylcarbamoyl) pyridin-2-yl)-N-(4-(3,3- dimethylureido)benzyl)- 7-isopropyl-1-methyl- 4,5,6,7-tetrahydro-1H- pyrazolo[4,3-c]pyridine- 3-carboxamide | 547.2 |
| 266 | Ex. 127 | 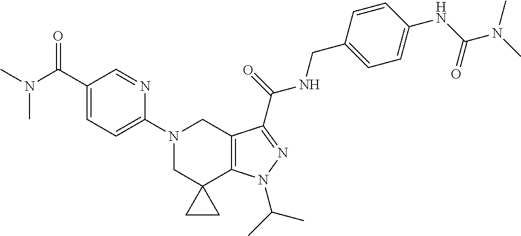 ¹H NMR* (600 MHz, (CD₃)₂SO) δ 8.35 (t, 1H), 8.23 (s, 1H), 8.21-8.20 (m, 1H), 7.65- 7.61 (m, 1H), 7.42-7.37 (m, 2H), 7.20- 7.15 (m, 2H), 6.90 (d, 1H), 4.81 (s, 2H), 4.35 (d, 2H), 3.98-3.90 (m, 1H), 3.73 (s, 2H), 3.03-2.93 (m, 6H), 2.91 (s, 6H), 1.38 (d, 6H), 1.30 (t, 2H), 1.09 (t, 2H). | 5′-(5- (dimethylcarbamoyl) pyridin-2-yl)-N-(4-(3,3- dimethylureido)benzyl)- 1′-isopropyl-1′,4′,5′,6′- tetrahydrospiro [cyclopropane- 1,7′-pyrazolo[4,3- c]pyridine]-3′- carboxamide | 559.6 |
| 267^{∧} | Ex. 128^{de} | 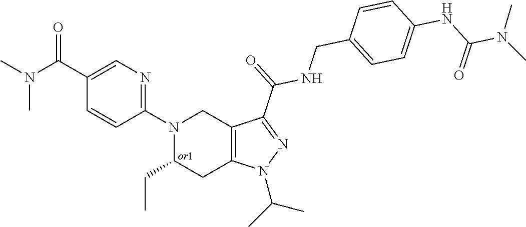 ¹H NMR (600 MHz, (CD₃)₂SO) δ 8.34 (t, 1H), 8.26-8.21 (m, 2H), 7.63 (dd, 1H), 7.42-7.36 (m, 2H), 7.21-7.15 (m, 2H), 6.86 (d, 1H), 5.22 (d, 1H), 5.03 (s, 1H), 4.58- 4.46 (m, 1H), 4.40-4.26 (m, 2H), 4.07 (d, 1H), 3.03-2.94 (m, 6H), 2.93-2.88 (m, 7H), 2.86-2.78 (m, 1H), 1.56-1.35 (m, 8H), 0.79 (t, 3H). | rel-(R or S)-5-(5- (dimethylcarbamoyl) pyridin-2-yl)-N-(4-(3,3- dimethylureido)benzyl)- 6-ethyl-1-isopropyl- 4,5,6,7-tetrahydro-1H- pyrazolo[4,3-c]pyridine- 3-carboxamide | 561.2 |
| 268^{∧} | Ex. 128^{de} | 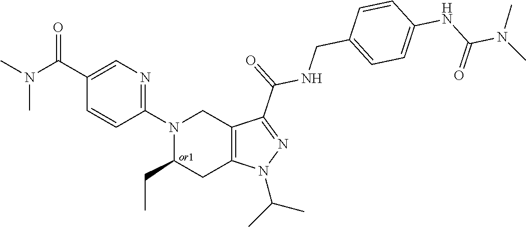 | rel-(R or S)-5-(5- (dimethylcarbamoyl) pyridin-2-yl)-N-(4-(3,3- dimethylureido)benzyl)- 6-ethyl-1-isopropyl- 4,5,6,7-tetrahydro-1H- pyrazolo[4,3-c]pyridine- 3-carboxamide | 561.2 |
| 269^{∧} | Ex. 113 | 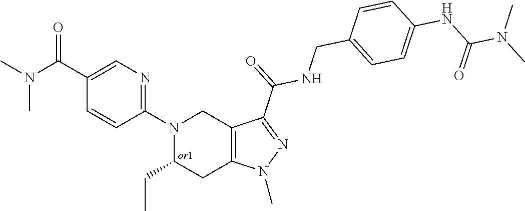 ¹H NMR (400 MHz, (CD₃)₂SO) δ 8.58- 8.44 (m, 1H), 8.32-8.20 (m, 2H), 7.73- 7.60 (m, 1H), 7.47-7.34 (m, 2H), 7.24- 7.11 (m, 2H), 6.94-6.83 (m, 1H), 5.38- 5.19 (m, 1H), 5.09-4.92 (m, 1H), 4.40- 4.26 (m, 2H), 4.13-3.98 (m, 1H), 3.84- 3.71 (m, 3H), 3.06-2.97 (m, 6H), 2.94- 2.89 (m, 6H), 2.89-2.65 (m, 2H), 1.59- 1.40 (m, 2H), 0.89-0.77 (m, 3H). | rel-(R or S)-5-(5- (dimethylcarbamoyl) pyridin-2-yl)-N-(4-(3,3- dimethylureido)benzyl)- 6-ethyl-1-methyl-4,5,6,7- tetrahydro-1H- pyrazolo[4,3-c]pyridine- 3-carboxamide | 533.3 |
| 270^{∧} | Ex. 113 | 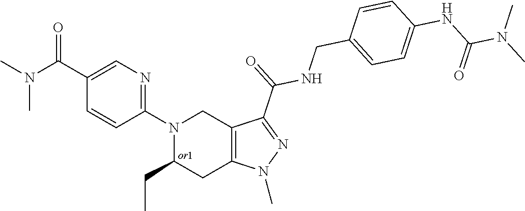 ¹H NMR (400 MHz, (CD₃)₂SO) δ 8.49 (t, 1H), 8.27-8.19 (m, 2H), 7.64 (dd, 1H), 7.41-7.35 (m, 2H), 7.17 (d, 2H), 6.87 (d, 1H), 5.32-5.20 (m, 1H), 5.08-4.92 (m, 1H), 4.40-4.25 (m, 2H), 4.05 (d, 1H), 3.77 (s, 3H), 2.98 (s, 6H), 2.93-2.85 (m, 7H), 2.82-2.74 (m, 1H), 1.59-1.40 (m, 2H), 0.81 (t, 3H). | rel-(R or S)-5-(5- (dimethylcarbamoyl) pyridin-2-yl)-N-(4-(3,3- dimethylureido)benzyl)- 6-ethyl-1-methyl-4,5,6,7- tetrahydro-1H- pyrazolo[4,3-c]pyridine- 3-carboxamide | 533.3 |
| 271^{∧} | Ex. 113 | 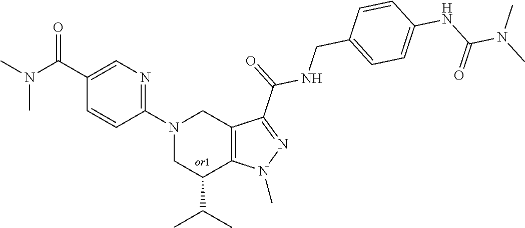 ¹H NMR (400 MHz, (CD₃)₂SO) δ 8.53 (t, 1H), 8.28 (d, 1H), 8.22 (s, 1H), 7.67 (dd, 1H), 7.42-7.35 (m, 2H), 7.20-7.14 (m, 2H), 6.79 (d, 1H), 4.99-4.79 (m, 2H), 4.40- 4.25 (m, 3H), 3.84 (s, 3H), 3.11-3.03 (m, 1H), 2.99 (s, 6H), 2.96-2.92 (m, 1H), 2.91- 2.88 (m, 6H), 1.95-1.80 (m, 1H), 1.00 (d, 3H), 0.88 (d, 3H). | rel-(R or S)-5-(5- (dimethylcarbamoyl) pyridin-2-yl)-N-(4-(3,3- dimethylureido)benzyl)- 7-isopropyl-1-methyl- 4,5,6,7-tetrahydro-1H- pyrazolo[4,3-c]pyridine- 3-carboxamide | 547.2 |
| 272^{∧} | Ex. 113 | 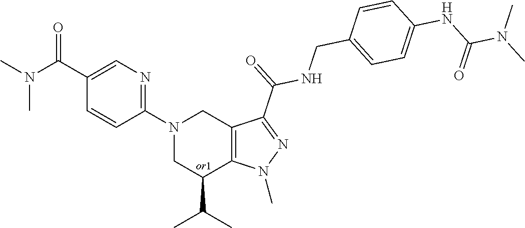 ¹H NMR (400 MHz, (CD₃)₂SO) δ 8.53 (t, 1H), 8.28 (d, 1H), 8.22 (s, 1H), 7.67 (dd, 1H), 7.42-7.35 (m, 2H), 7.21-7.13 (m, 2H), 6.79 (d, 1H), 5.02-4.80 (m, 2H), 4.41- 4.25 (m, 3H), 3.84 (s, 3H), 3.12-3.03 (m, 1H), 2.99 (s, 6H), 2.95-2.92 (m, 1H), 2.91- 2.88 (m, 6H), 1.96-1.84 (m, 1H), 1.00 (d, 3H), 0.88 (d, 3H). | rel-(R or S)-5-(5- (dimethylcarbamoyl) pyridin-2-yl)-N-(4-(3,3- dimethylureido)benzyl)- 7-isopropyl-1-methyl- 4,5,6,7-tetrahydro-1H- pyrazolo[4,3-c]pyridine- 3-carboxamide | 547.3 |
| 273^{∧} | Ex. 77 | 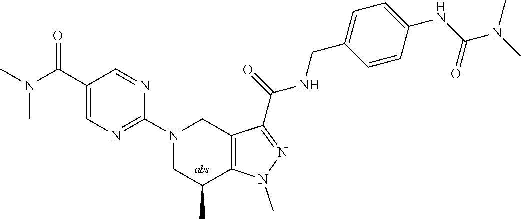 ¹H NMR (400 MHz, (CD₃)₂SO) δ 8.56- 8.44 (m, 3H), 8.22 (s, 1H), 7.42-7.35 (m, 2H), 7.19-7.13 (m, 2H), 5.40 (d, 1H), 4.63 (dd, 1H), 4.47-4.25 (m, 3H), 3.82 (s, 3H), 3.51 (dd, 1H), 3.27-3.19 (m, 1H), 3.05- 2.95 (m, 6H), 2.91 (s, 6H), 1.16 (d, 3H). | (S)-5-(5- (dimethylcarbamoyl) pyrimidin-2-yl)-N-(4-(3,3- dimethylureido)benzyl)- 1,7-dimethyl-4,5,6,7- tetrahydro-1H- pyrazolo[4,3-c]pyridine- 3-carboxamide | 520.3 |
| 274^{∧} | Ex. 5 | 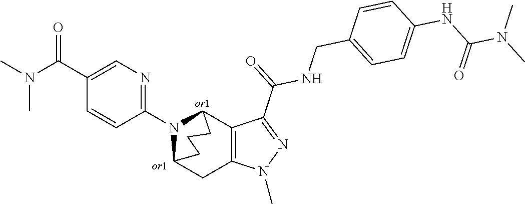 ¹H NMR (400 MHz, CD₃OD) δ 8.22 (d, 1H), 7.64-7.56 (m, 1H), 7.36-7.29 (m, 2H), 7.28-7.22 (m, 2H), 6.86 (d, 1H), 5.90- 5.84 (m, 1H), 5.22-5.15 (m, 1H), 4.47 (s, 2H), 3.77-3.69 (m, 3H), 3.16-2.97 (m, 13H), 2.69-2.56 (m, 1H), 1.97-1.76 (m, 4H), 1.58-1.34 (m, 2H). | rel-(4R,8S)-10-(5- (dimethylcarbamoyl) pyridin-2-yl)-N-(4-(3,3- dimethylureido)benzyl)- 1-methyl-4,5,6,7,8,9- hexahydro-1H-4,8- epiminocycloocta[c] pyrazole-3-carboxamide | 545.3 |
| 275^{∧} | Ex. 5 | 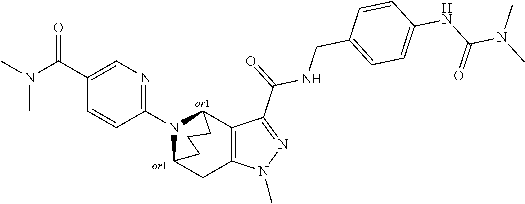 ¹H NMR (400 MHz, CD₃OD) δ 8.23 (d, 1H), 7.62-7.58 (m, 1H), 7.35-7.30 (m, 2H), 7.28-7.23 (m, 2H), 6.86 (d, 1H), 5.87 (s, 1H), 5.24-5.13 (m, 1H), 4.47 (s, 2H), 3.74- 3.72 (m, 3H), 3.17-2.96 (m, 13H), 2.63 (d, 1H), 1.97-1.75 (m, 4H), 1.60-1.35 (m, 2H). | rel-(4R,8S)-10-(5- (dimethylcarbamoyl) pyridin-2-yl)-N-(4-(3,3- dimethylureido)benzyl)- 1-methyl-4,5,6,7,8,9- hexahydro-1H-4,8- epiminocycloocta[c] pyrazole-3-carboxamide | 545.3 |
| 276^{∧} | Ex. 113^{df} | 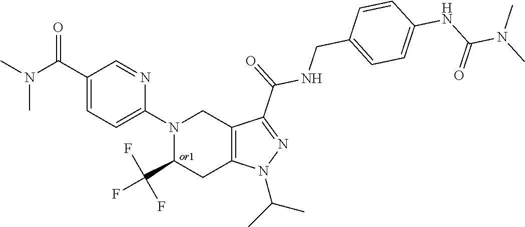 ¹H NMR (600 MHz, (CD₃)₂SO) δ 8.42 (t, 1H), 8.33 (d, 1H), 8.23 (s, 1H), 7.78 (dd, 1H), 7.42-7.37 (m, 2H), 7.21-7.16 (m, 2H), 7.01 (d, 1H), 6.31-6.22 (m, 1H), 5.04 (d, 1H), 4.65-4.56 (m, 1H), 4.39-4.23 (m, 3H), 3.29-3.19 (m, 2H), 3.03-2.95 (m, 6H), 2.90 (s, 6H), 1.41 (dd, 6H). | rel-(R or S)-5-(5- (dimethylcarbamoyl) pyridin-2-yl)-N-(4-(3,3- dimethylureido)benzyl)- 1-isopropyl-6- (trifluoromethyl)-4,5,6,7- tetrahydro-1H- pyrazolo[4,3-c]pyridine- 3-carboxamide | 601.2 |
| 277^{∧} | Ex. 113^{df} | 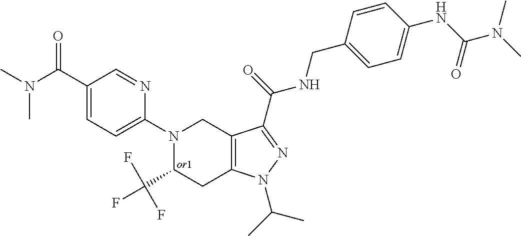 ¹H NMR (600 MHz, (CD₃)₂SO) δ 8.42 (t, 1H), 8.33-8.32 (m, 1H), 8.23 (s, 1H), 7.78 (dd, 1H), 7.41-7.37 (m, 2H), 7.21-7.17 (m, 2H), 7.01 (d, 1H), 6.32-6.21 (m, 1H), 5.04 (d, 1H), 4.66-4.57 (m, 1H), 4.39- 4.24 (m, 3H), 3.31-3.19 (m, 2H), 3.03- 2.96 (m, 6H), 2.90 (s, 6H), 1.41 (dd, 6H). | rel-(R or S)-5-(5- (dimethylcarbamoyl) pyridin-2-yl)-N-(4-(3,3- dimethylureido)benzyl)- 1-isopropyl-6- (trifluoromethyl)-4,5,6,7- tetrahydro-1H- pyrazolo[4,3-c]pyridine- 3-carboxamide | 601.2 |
| 278 | Ex. 113 | 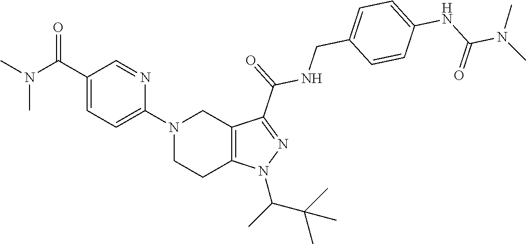 ¹H NMR (400 MHz, (CD₃)₂SO) δ 8.30- 8.19 (m, 3H), 7.64 (dd, 1H), 7.42-7.37 (m, 2H), 7.21-7.15 (m, 2H), 6.88 (d, 1H), 4.85- 4.60 (m, 2H), 4.44-4.27 (m, 2H), 4.13- 3.99 (m, 2H), 3.85-3.76 (m, 1H), 2.97 (s, 6H), 2.91 (s, 6H), 2.84-2.75 (m, 2H), 1.41 (d, 3H), 0.89 (s, 9H). | 1-(3,3-dimethylbutan-2- yl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 575.5 |
| 279 | Ex. 43 | 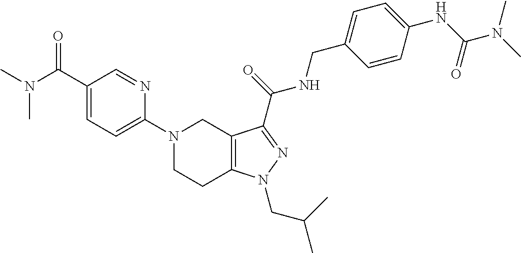 ¹H NMR (400 MHz, (CD₃)₂SO) δ 8.42 (t, 1H), 8.25-8.20 (m, 2H), 7.64 (dd, 1H), 7.42-7.35 (m, 2H), 7.20-7.14 (m, 2H), 6.88 (d, 1H), 4.74 (s, 2H), 4.33 (d, 2H), 3.95 (t, 2H), 3.85 (d, 2H), 2.97 (s, 6H), 2.91 (s, 6H), 2.83-2.74 (m, 2H), 2.22-2.05 (m, 1H), 0.85 (d, 6H). | 5-(5- (dimethylcarbamoyl) pyridin-2-yl)-N-(4-(3,3- dimethylureido)benzyl)- 1-isobutyl-4,5,6,7- tetrahydro-1H- pyrazolo[4,3-c]pyridine- 3-carboxamide | 547.3 |
| 280^{∧} | Ex. 252 | 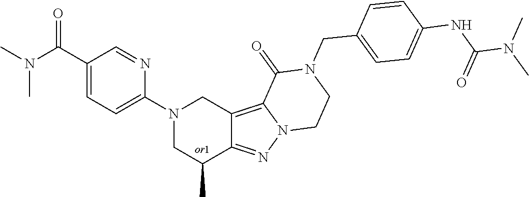 ¹H NMR (600 MHz, (CD₃)₂SO) δ 8.28 (s, 1H), 8.25-8.24 (m, 1H), 7.64 (dd, 1H), 7.46-7.42 (m, 2H), 7.21-7.17 (m, 2H), 6.93 (d, 1H), 5.08 (d, 1H), 4.69-4.47 (m, 3H), 4.34-4.20 (m, 3H), 3.67 (t, 2H), 3.31- 3.25 (m, 1H), 3.04-2.94 (m, 7H), 2.91 (s, 6H), 1.25 (d, 3H). | rel-(R or S)-6-(9-(4-(3,3- dimethylureido)benzyl)- 4-methyl-10-oxo- 3,4,7,8,9,10- hexahydropyrido[4′,3′:3, 4]pyrazolo[1,5- a]pyrazin-2(1H)-yl)-N,N- dimethylnicotinamide | 531.2 |
| 281^{∧} | Ex. 252 | 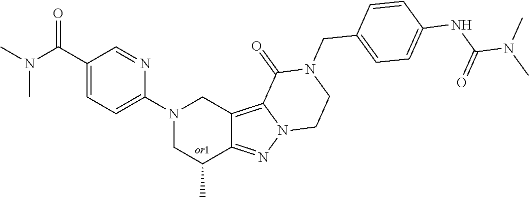 ¹H NMR (600 MHz, (CD₃)₂SO) δ 8.28 (s, 1H), 8.25 (d, 1H), 7.64 (dd, 1H), 7.46-7.42 (m, 2H), 7.21-7.17 (m, 2H), 6.93 (d, 1H), 5.08 (d, 1H), 4.67-4.49 (m, 3H), 4.34- 4.19 (m, 3H), 3.67 (t, 2H), 3.31-3.25 (m, 1H), 3.04-2.95 (m, 7H), 2.91 (d, 6H), 1.25 (d, 3H). | rel-(R or S)-6-(9-(4-(3,3- dimethylureido)benzyl)- 4-methyl-10-oxo- 3,4,7,8,9,10- hexahydropyrido[4′,3′:3, 4]pyrazolo[1,5- a]pyrazin-2(1H)-yl)-N,N- dimethylnicotinamide | 531.2 |
| 282 | Ex. 3^{l} | 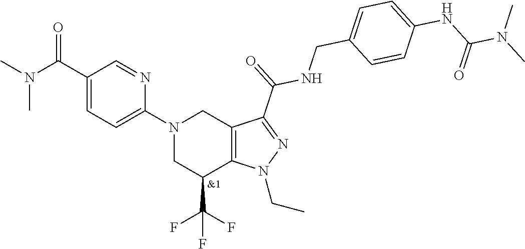 ¹H NMR (600 MHz, (CD₃)₂SO) δ 8.62 (t, 1H), 8.28-8.27 (m, 1H), 8.22 (s, 1H), 7.69 (dd, 1H), 7.41-7.37 (m, 2H), 7.20-7.15 (m, 2H), 6.84 (d, 1H), 5.27 (d, 1H), 5.08 (d, 1H), 4.51-4.42 (m, 1H), 4.40-4.11 (m, 5H), 3.32-3.26 (m, 1H), 3.03-2.94 (m, 6H), 2.90 (s, 6H), 1.42 (t, 3H). | rac-(R)-5-(5- (dimethylcarbamoyl) pyridin-2-yl)-N-(4-(3,3- dimethylureido)benzyl)- 1-ethyl-7- (trifluoromethyl)-4,5,6,7- tetrahydro-1H- pyrazolo[4,3-c]pyridine- 3-carboxamide | 587.4 |
| 283 | Ex. 125 | 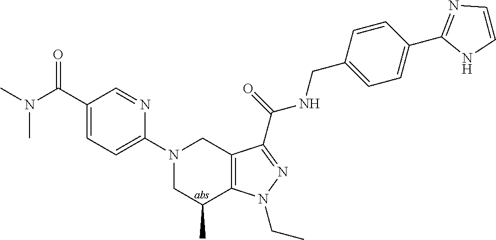 [SMILES: CCn1nc(C(=O)NCc2ccc(-c3ncc[nH]3)cc2)c2c1[C@@H](C)CN(c1ccc(C(=O)N(C)C)cn1)C2] ¹H NMR* (600 MHz, (CD₃)₂SO) δ 14.66 (s, 1H), 8.74 (t, 1H), 8.25-8.20 (m, 1H), 7.98- 7.92 (m, 2H), 7.82 (s, 2H), 7.64 (dd, 1H), 7.58-7.55 (m, 2H), 6.91 (d, 1H), 5.06 (d, 1H), 4.58-4.42 (m, 2H), 4.37-4.22 (m, 2H), 4.19-4.07 (m, 2H), 3.41 (dd, 1H), 3.27-3.21 (m, 1H), 3.01-2.94 (m, 6H), 1.41 (t, 3H), 1.21 (d, 3H). | (S)-N-(4-(1H-imidazol-2- yl)benzyl)-5-(5- (dimethylcarbamoyl) pyridin-2-yl)-1-ethyl-7- methyl-4,5,6,7- tetrahydro-1H- pyrazolo[4,3-c]pyridine- 3-carboxamide | 513.4 |
| 284 | Ex. 125 | 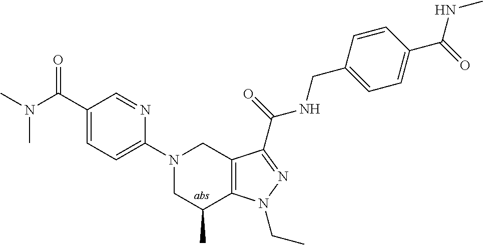 ¹H NMR* (600 MHz, (CD₃)₂SO) δ 8.66 (t, 1H), 8.36 (q, 1H), 8.22 (d, 1H), 7.79-7.75 (m, 2H), 7.69-7.64 (m, 1H), 7.39-7.34 (m, 2H), 6.95 (d, 1H), 5.04 (d, 1H), 4.51- 4.38 (m, 2H), 4.37-4.27 (m, 2H), 4.19- 4.05 (m, 2H), 3.42 (dd, 1H), 3.27-3.20 (m, 1H), 3.00-2.94 (m, 6H), 2.77 (d, 3H), 1.40 (t, 3H), 1.21 (d, 3H). | (S)-5-(5- (dimethylcarbamoyl) pyridin-2-yl)-1-ethyl-7- methyl-N-(4- (methylcarbamoyl)benzyl)- 4,5,6,7-tetrahydro-1H- pyrazolo[4,3-c]pyridine- 3-carboxamide | 504.4 |
| 285^{∧} | Ex. 113 | 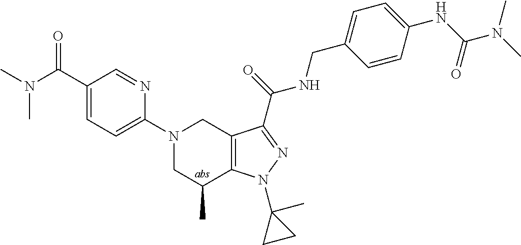 ¹H NMR (400 MHz, (CD₃)₂SO) δ 8.46 (t, 1H), 8.27-8.19 (m, 2H), 7.64 (dd, 1H), 7.43-7.33 (m, 2H), 7.21-7.15 (m, 2H), 6.91-6.86 (m, 1H), 4.78 (d, 1H), 4.57 (d, 1H), 4.40-4.24 (m, 2H), 4.01 (dd, 1H), 3.67 (dd, 1H), 2.98 (s, 6H), 2.91 (s, 6H), 1.54 (s, 3H), 1.50-1.42 (m, 1H), 1.35 (d, 3H), 1.32-1.22 (m, 2H), 1.12-1.07 (m, 1H), 1.02-0.97 (m, 1H). | (S)-5-(5- (dimethylcarbamoyl) pyridin-2-yl)-N-(4-(3,3- dimethylureido)benzyl)- 7-methyl-1-(1- methylcyclopropyl)- 4,5,6,7-tetrahydro-1H- pyrazolo[4,3-c]pyridine- 3-carboxamide | 559.3 |
| 286 | Ex. 286 | 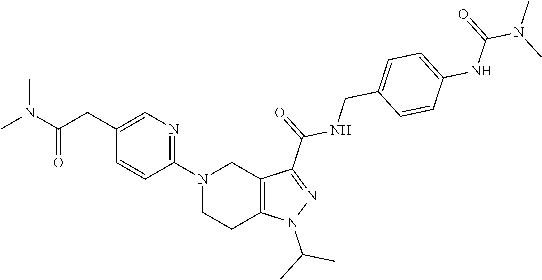 ¹H NMR (400 MHz, (CD₃)₂SO) δ 8.33- 8.16 (m, 2H), 7.91 (s, 1H), 7.35 (d, 3H), 7.14 (d, 2H), 6.79 (d, 1H), 4.60 (s, 2H), 4.50- 4.37 (m, 1H), 4.35-4.27 (m, 2H), 3.89- 3.76 (m, 2H), 3.53-3.44 (m, 2H), 2.97 (s, 3H), 2.87 (s, 6H), 2.80-2.70 (m, 5H), 1.35 (d, 6H). | 5-(5-(2-(dimethylamino)- 2-oxoethyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 547.3 |
| 287^{∧} | Ex. 113 | 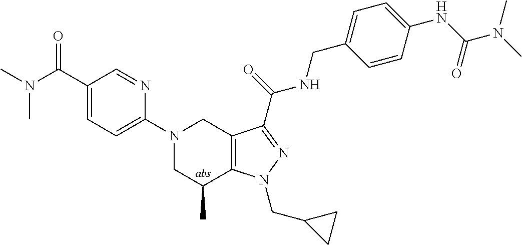 ¹H NMR (400 MHz, (CD₃)₂SO) δ 8.44- 8.35 (m, 1H), 8.23-8.16 (m, 2H), 7.64- 7.57 (m, 1H), 7.34 (d, 2H), 7.13 (d, 2H), 6.85 (d, 1H), 5.02 (d, 1H), 4.43-4.19 (m, 4H), 4.07-3.97 (m, 1H), 3.88-3.77 (m, 1H), 3.24-3.14 (m, 2H), 2.94 (s, 6H), 2.86 (s, 6H), 1.30-1.12 (m, 4H), 0.53-0.43 (m, 2H), 0.40-0.32 (m, 2H). | (S)-1- (cyclopropylmethyl)-5- (5- (dimethylcarbamoyl) pyridin-2-yl)-N-(4-(3,3- dimethylureido)benzyl)- 7-methyl-4,5,6,7- tetrahydro-1H- pyrazolo[4,3-c]pyridine- 3-carboxamide | 559.3 |
| 288 | Ex. 193 | 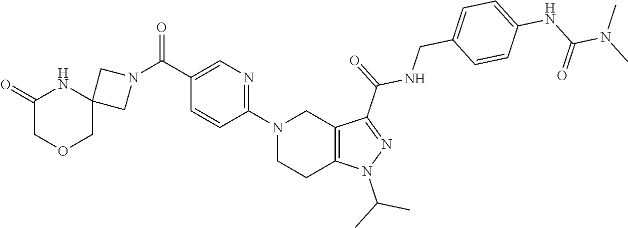 [SMILES: CC(C)n1nc(C(=O)NCc2ccc(NC(=O)N(C)C)cc2)c2c1CCN(c1ccc(C(=O)N3CC4(COCC(=O)N4)C3)cn1)C2] | N-(4-(3,3- dimethylureido)benzyl)- 1-isopropyl-5-(5-(6-oxo- 8-oxa-2,5- diazaspiro[3.5]nonane- 2-carbonyl)pyridin-2-yl)- 4,5,6,7-tetrahydro-1H- pyrazolo[4,3-c]pyridine- 3-carboxamide | 630.6 |
| 289 | Ex. 193 | 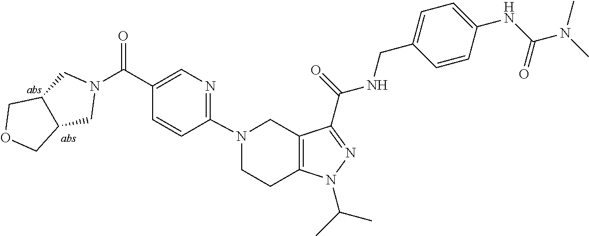 [SMILES: CC(C)n1nc(C(=O)NCc2ccc(NC(=O)N(C)C)cc2)c2c1CCN(c1ccc(C(=O)N3C[C@H]4COC[C@H]4C3)cn1)C2] | N-(4-(3,3- dimethylureido)benzyl)- 5-(5-((3aR,6aS)- hexahydro-1H-furo[3,4- c]pyrrole-5- carbonyl)pyridin-2-yl)-1- isopropyl-4,5,6,7- tetrahydro-1H- pyrazolo[4,3-c]pyridine- 3-carboxamide | 601.7 |
| 290 | Ex. 193 | 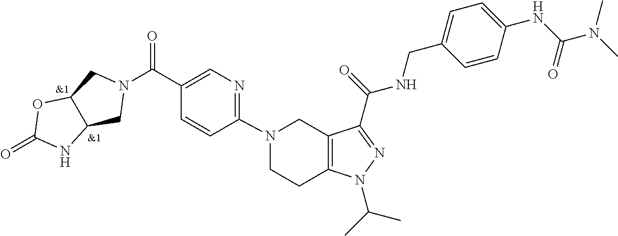 [SMILES: CC(C)n1nc(C(=O)NCc2ccc(NC(=O)N(C)C)cc2)c2c1CCN(c1ccc(C(=O)N3C[C@@H]4OC(=O)N[C@@H]4C3)cn1)C2] | rac-N-(4-(3,3- dimethylureido)benzyl)- 1-isopropyl-5-(5- ((3aR,6aS)-2- oxohexahydro-2H- pyrrolo[3,4-d]oxazole-5- carbonyl)pyridin-2-yl)- 4,5,6,7-tetrahydro-1H- pyrazolo[4,3-c]pyridine- 3-carboxamide | 616.6 |
| 291 | Ex. 193 | 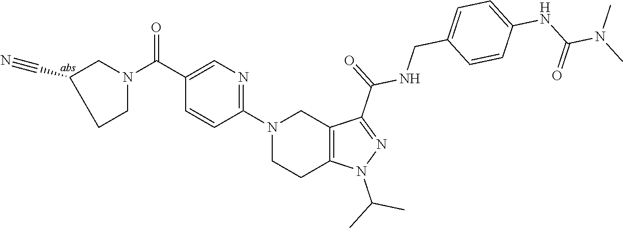 [SMILES: CC(C)n1nc(C(=O)NCc2ccc(NC(=O)N(C)C)cc2)c2c1CCN(c1ccc(C(=O)N3CC[C@H](C#N)C3)cn1)C2] | (S)-5-(5-(3- cyanopyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 584.6 |
| 292 | Ex. 193 | 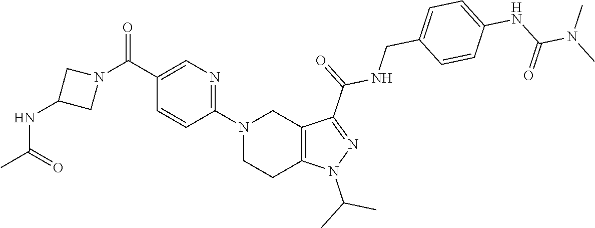 [SMILES: CC(=O)NC1CN(C(=O)c2ccc(N3CCc4c(c(C(=O)NCc5ccc(NC(=O)N(C)C)cc5)nn4C(C)C)C3)nc2)C1] | 5-(5-(3- acetamidoazetidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 602.6 |
| 293 | Ex. 193 | 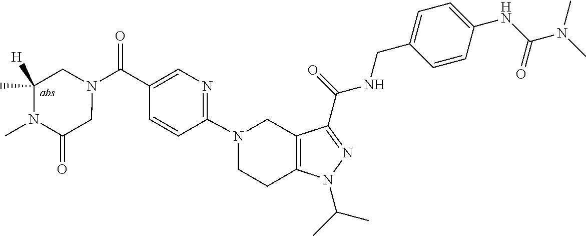 | (R)-5-(5-(3,4-dimethyl-5- oxopiperazine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 616.6 |
| 294 | Ex. 193 | 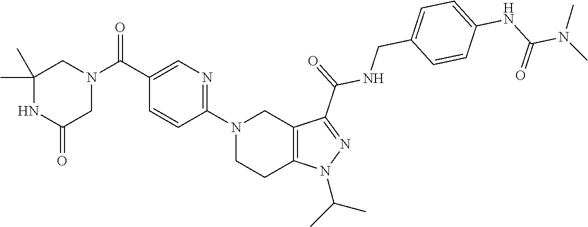 | 5-(5-(3,3-dimethyl-5- oxopiperazine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 616.6 |
| 295 | Ex. 193 | 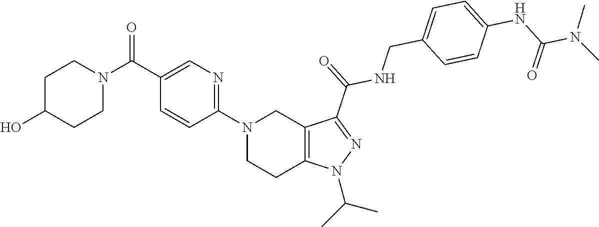 [SMILES: CC(C)n1nc(C(=O)NCc2ccc(NC(=O)N(C)C)cc2)c2c1CCN(c1ccc(C(=O)N3CCC(O)CC3)cn1)C2] | N-(4-(3,3- dimethylureido)benzyl)- 5-(5-(4- hydroxypiperidine-1- carbonyl)pyridin-2-yl)-1- isopropyl-4,5,6,7- tetrahydro-1H- pyrazolo[4,3-c]pyridine- 3-carboxamide | 589.6 |
| 296 | Ex. 193 | 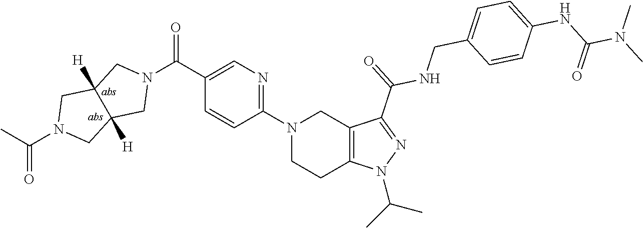 [SMILES: CC(=O)N1C[C@@H]2CN(C(=O)c3ccc(N4CCc5c(c(C(=O)NCc6ccc(NC(=O)N(C)C)cc6)nn5C(C)C)C4)nc3)C[C@@H]2C1] | 5-(5-((3aR,6aS)-5- acetyloctahydropyrrolo[3, 4-c]pyrrole-2- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 642.6 |
| 297 | Ex. 193 | 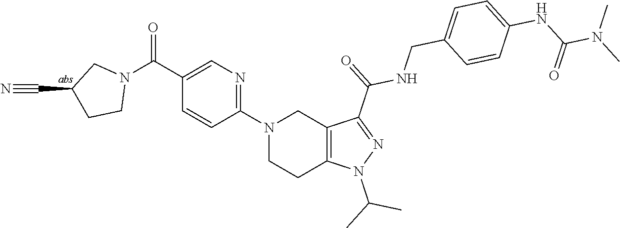 [SMILES: CC(C)n1nc(C(=O)NCc2ccc(NC(=O)N(C)C)cc2)c2c1CCN(c1ccc(C(=O)N3CC[C@@H](C#N)C3)cn1)C2] | (R)-5-(5-(3- cyanopyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 584.6 |
| 298 | Ex. 193 | 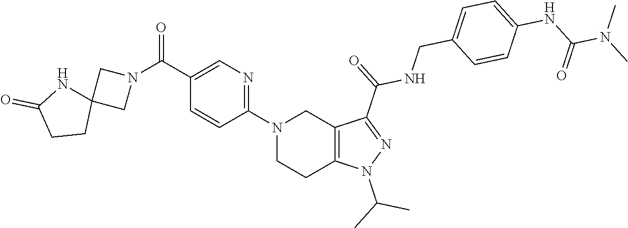 [SMILES: CC(C)n1nc(C(=O)NCc2ccc(NC(=O)N(C)C)cc2)c2c1CCN(c1ccc(C(=O)N3CC4(CCC(=O)N4)C3)cn1)C2] | N-(4-(3,3- dimethylureido)benzyl)- 1-isopropyl-5-(5-(6-oxo- 2,5- diazaspiro[3.4]octane-2- carbonyl)pyridin-2-yl)- 4,5,6,7-tetrahydro-1H- pyrazolo[4,3-c]pyridine- 3-carboxamide | 614.6 |
| 299 | Ex. 193 | 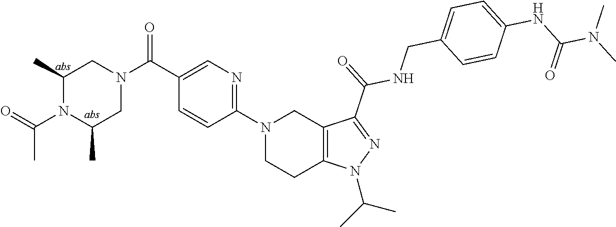 [SMILES: CC(=O)N1[C@H](C)CN(C(=O)c2ccc(N3CCc4c(c(C(=O)NCc5ccc(NC(=O)N(C)C)cc5)nn4C(C)C)C3)nc2)C[C@@H]1C] | 5-(5-((3S,5R)-4-acetyl- 3,5-dimethylpiperazine- 1-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 644.7 |
| 300 | Ex. 193 | 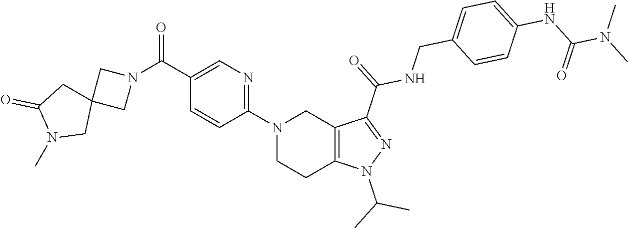 [SMILES: CC(C)n1nc(C(=O)NCc2ccc(NC(=O)N(C)C)cc2)c2c1CCN(c1ccc(C(=O)N3CC4(CC(=O)N(C)C4)C3)cn1)C2] | N-(4-(3,3- dimethylureido)benzyl)- 1-isopropyl-5-(5-(6- methyl-7-oxo-2,6- diazaspiro[3.4]octane-2- carbonyl)pyridin-2-yl)- 4,5,6,7-tetrahydro-1H- pyrazolo[4,3-c]pyridine- 3-carboxamide | 628.6 |
| 301 | Ex. 125 | 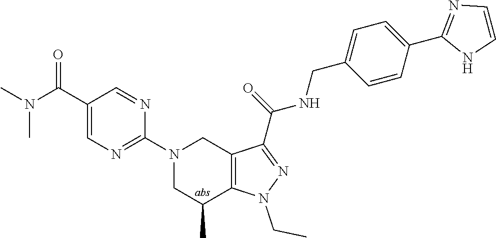 [SMILES: CCn1nc(C(=O)NCc2ccc(-c3ncc[nH]3)cc2)c2c1[C@@H](C)CN(c1ncc(C(=O)N(C)C)cn1)C2] ¹H NMR* (600 MHz, (CD₃)₂SO) δ 14.70 (s, 1H), 8.74 (t, 1H), 8.51 (s, 2H), 7.97-7.93 (m, 2H), 7.79-7.78 (m, 2H), 7.57-7.54 (m, 2H), 5.40 (d, 1H), 4.68 (dd, 1H), 4.55- 4.43 (m, 2H), 4.40-4.35 (m, 1H), 4.20- 4.04 (m, 2H), 3.48 (dd, 1H), 3.29-3.22 (m, 1H), 3.06-2.91 (m, 6H), 1.40 (t, 3H), 1.17 (d, 3H). | (S)-N-(4-(1H-imidazol-2- yl)benzyl)-5-(5- (dimethylcarbamoyl) pyrimidin-2-yl)-1-ethyl-7- methyl-4,5,6,7- tetrahydro-1H- pyrazolo[4,3-c]pyridine- 3-carboxamide | 514.4 |
| 302 | Ex. 126 | 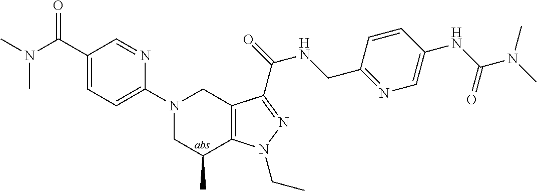 ¹H NMR* (600 MHz, (CD₃)₂SO) δ 8.96- 8.90 (m, 1H), 8.84-8.80 (m, 1H), 8.75- 8.71 (m, 1H), 8.24-8.17 (m, 2H), 7.65 (dd, 1H), 7.59 (s, 1H), 6.92 (d, 1H), 5.05 (d, 1H), 4.64-4.52 (m, 2H), 4.37-4.25 (m, 2H), 4.20-4.07 (m, 2H), 3.41 (dd, 1H), 3.28- 3.21 (m, 1H), 3.00-2.97 (m, 6H), 2.96- 2.92 (m, 6H), 1.41 (t, 3H), 1.22 (d, 3H). | (S)-5-(5- (dimethylcarbamoyl) pyridin-2-yl)-N-((5-(3,3- dimethylureido)pyridin- 2-yl)methyl)-1-ethyl-7- methyl-4,5,6,7- tetrahydro-1H- pyrazolo[4,3-c]pyridine- 3-carboxamide | 534.4 |
| 303^{∧} | Ex. 3^{l} | 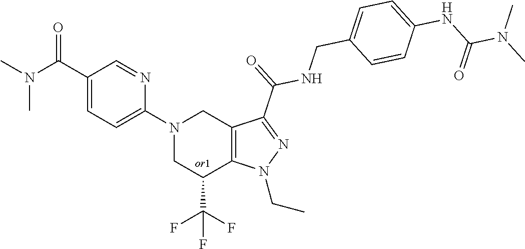 ¹H NMR (600 MHz, (CD₃)₂SO) δ 8.63 (t, 1H), 8.28 (d, 1H), 8.24 (s, 1H), 7.70 (dd, 1H), 7.42-7.37 (m, 2H), 7.21-7.16 (m, 2H), 6.85 (d, 1H), 5.28 (d, 1H), 5.09 (d, 1H), 4.52-4.46 (m, 1H), 4.41-4.32 (m, 2H), 4.31-4.26 (m, 1H), 4.24-4.14 (m, 2H), 3.31-3.27 (m, 1H), 3.04-2.96 (m, 6H), 2.91 (s, 6H), 1.43 (t, 3H). | rel-(R or S)-5-(5- (dimethylcarbamoyl) pyridin-2-yl)-N-(4-(3,3- dimethylureido)benzyl)- 1-ethyl-7- (trifluoromethyl)-4,5,6,7- tetrahydro-1H- pyrazolo[4,3-c]pyridine- 3-carboxamide | 587.2 |
| 304^{∧} | Ex. 3^{l} | 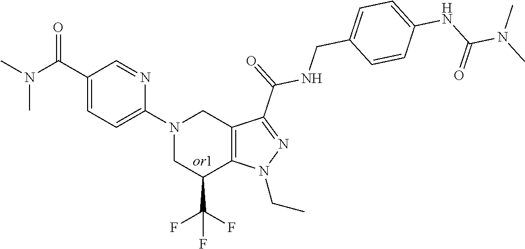 ¹H NMR (600 MHz, (CD₃)₂SO) δ 8.62 (t, 1H), 8.28-8.26 (m, 1H), 8.23 (s, 1H), 7.69 (dd, 1H), 7.41-7.37 (m, 2H), 7.20-7.16 (m, 2H), 6.84 (d, 1H), 5.27 (d, 1H), 5.08 (d, 1H), 4.51-4.45 (m, 1H), 4.39-4.31 (m, 2H), 4.30-4.24 (m, 1H), 4.23-4.13 (m, 2H), 3.30-3.26 (m, 1H), 3.03-2.94 (m, 6H), 2.90 (s, 6H), 1.42 (t, 3H). | rel-(R or S)-5-(5- (dimethylcarbamoyl) pyridin-2-yl)-N-(4-(3,3- dimethylureido)benzyl)- 1-ethyl-7- (trifluoromethyl)-4,5,6,7- tetrahydro-1H- pyrazolo[4,3-c]pyridine- 3-carboxamide | 587.2 |
| 305 | Ex. 125 | 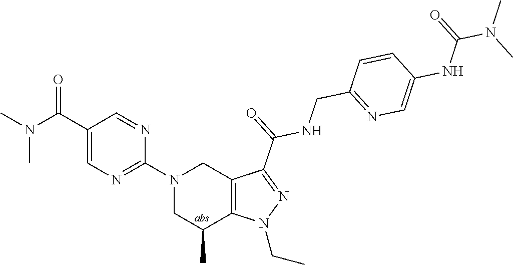 ¹H NMR* (600 MHz, (CD₃)₂SO) δ 8.94- 8.80 (m, 1H), 8.77 (s, 1H), 8.70-8.64 (m, 1H), 8.57-8.49 (m, 2H), 8.16-8.10 (m, 1H), 7.54-7.48 (m, 1H), 5.41 (d, 1H), 4.68 (dd, 1H), 4.64-4.49 (m, 2H), 4.37 (d, 1H), 4.19-4.07 (m, 2H), 3.48 (dd, 1H), 3.30- 3.23 (m, 1H), 3.05-2.97 (m, 6H), 2.96- 2.92 (m, 6H), 1.44-1.38 (m, 3H), 1.17 (d, 3H). | (S)-5-(5- (dimethylcarbamoyl) pyrimidin-2-yl)-N-((5-(3,3- dimethylureido)pyridin- 2-yl)methyl)-1-ethyl-7- methyl-4,5,6,7- tetrahydro-1H- pyrazolo[4,3-c]pyridine- 3-carboxamide | 535.4 |
| 306 | Ex. 125 | 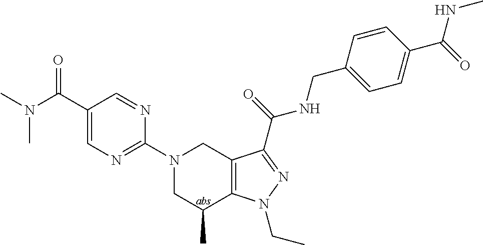 ¹H NMR (600 MHz, (CD₃)₂SO) δ 8.65 (t, 1H), 8.51 (s, 2H), 8.36 (q, 1H), 7.79-7.75 (m, 2H), 7.38-7.34 (m, 2H), 5.40 (d, 1H), 4.67 (dd, 1H), 4.51-4.34 (m, 3H), 4.17- 4.05 (m, 2H), 3.48 (dd, 1H), 3.28-3.22 (m, 1H), 3.03-2.96 (m, 6H), 2.77 (d, 3H), 1.39 (t, 3H), 1.16 (d, 3H). | (S)-5-(5- (dimethylcarbamoyl) pyrimidin-2-yl)-1-ethyl-7- methyl-N-(4- (methylcarbamoyl)benzyl)- 4,5,6,7-tetrahydro-1H- pyrazolo[4,3-c]pyridine- 3-carboxamide | 505.4 |
| 307^{∧} | Ex. 113 | 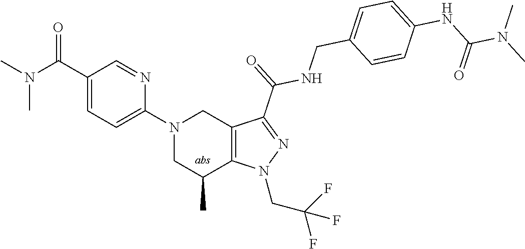 ¹H NMR (400 MHz, (CD₃)₂SO) δ 8.50 (t, 1H), 8.26-8.20 (m, 2H), 7.64 (dd, 1H), 7.40-7.35 (m, 2H), 7.19-7.14 (m, 2H), 6.91 (d, 1H), 5.23-5.03 (m, 3H), 4.52- 4.21 (m, 4H), 3.29-3.21 (m, 2H), 2.98 (s, 6H), 2.90 (s, 6H), 1.20 (d, 3H). | (S)-5-(5- (dimethylcarbamoyl) pyridin-2-yl)-N-(4-(3,3- dimethylureido)benzyl)- 7-methyl-1-(2,2,2- trifluoroethyl)-4,5,6,7- tetrahydro-1H- pyrazolo[4,3-c]pyridine- 3-carboxamide | 587.3 |
| 308 | Ex. 39 | 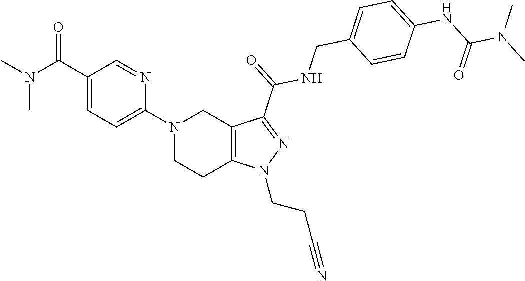 ¹H NMR (400 MHz, (CD₃)₂SO) δ 8.48- 8.41 (m, 1H), 8.26-8.21 (m, 2H), 7.67- 7.61 (m, 1H), 7.41-7.36 (m, 2H), 7.19- 7.14 (m, 2H), 6.93-6.88 (m, 1H), 4.76 (s, 2H), 4.38-4.31 (m, 4H), 3.99-3.92 (m, 2H), 3.09-3.04 (m, 2H), 2.97 (s, 6H), 2.91 (s, 8H). | 1-(2-cyanoethyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 544.2 |
| 309 | Ex. 39 | 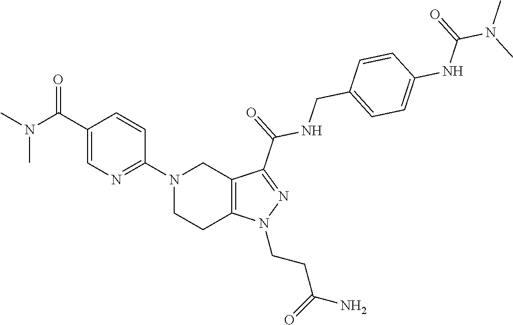 ¹H NMR (400 MHz, (CD₃)₂SO) δ 8.41 (t, 1H), 8.25-8.21 (m, 2H), 7.64 (dd, 1H), 7.41-7.34 (m, 3H), 7.20-7.14 (m, 2H), 6.93-6.85 (m, 2H), 4.74 (s, 2H), 4.34 (d, 2H), 4.21 (t, 2H), 3.97-3.90 (m, 2H), 2.99- 2.95 (m, 6H), 2.91 (s, 6H), 2.86-2.81 (m, 2H), 2.62 (t, 2H). | 1-(3-amino-3- oxopropyl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 562.3 |
| 310^{∧} | Ex. 2^{bd} | 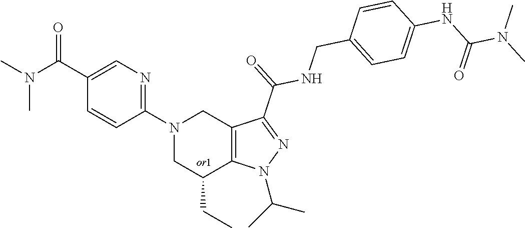 ¹H NMR (600 MHz, (CD₃)₂SO) δ 8.38- 8.33 (m, 1H), 8.27-8.21 (m, 2H), 7.67- 7.63 (m, 1H), 7.42-7.37 (m, 2H), 7.21- 7.16 (m, 2H), 6.84-6.81 (m, 1H), 5.02 (d, 1H), 4.82 (d, 1H), 4.57-4.48 (m, 1H), 4.40- 4.29 (m, 2H), 4.20 (d, 1H), 3.18-3.11 (m, 1H), 3.02-2.96 (m, 7H), 2.92-2.89 (m, 6H), 1.58-1.50 (m, 1H), 1.48-1.41 (m, 4H), 1.40-1.37 (m, 3H), 1.07-1.02 (m, 3H). | rel-(R or S)-5-(5- (dimethylcarbamoyl) pyridin-2-yl)-N-(4-(3,3- dimethylureido)benzyl)- 7-ethyl-1-isopropyl- 4,5,6,7-tetrahydro-1H- pyrazolo[4,3-c]pyridine- 3-carboxamide | 561.3 |
| 311^{∧} | Ex. 2^{bd} | 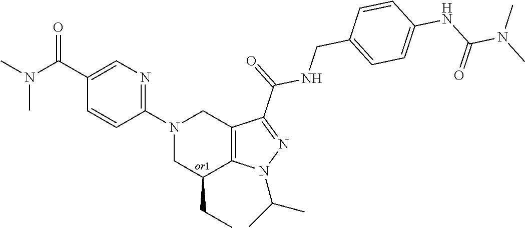 ¹H NMR (600 MHz, (CD₃)₂SO) δ 8.35 (t, 1H), 8.26-8.25 (m, 1H), 8.22 (s, 1H), 7.65 (dd, 1H), 7.41-7.37 (m, 2H), 7.20-7.15 (m, 2H), 6.82 (d, 1H), 5.02 (d, 1H), 4.82 (d, 1H), 4.56-4.48 (m, 1H), 4.39-4.30 (m, 2H), 4.22-4.17 (m, 1H), 3.20-3.09 (m, 1H), 3.03-2.94 (m, 7H), 2.90 (s, 6H), 1.58- 1.49 (m, 1H), 1.48-1.41 (m, 4H), 1.40- 1.37 (m, 3H), 1.04 (t, 3H). | rel-(R or S)-5-(5- (dimethylcarbamoyl) pyridin-2-yl)-N-(4-(3,3- dimethylureido)benzyl)- 7-ethyl-1-isopropyl- 4,5,6,7-tetrahydro-1H- pyrazolo[4,3-c]pyridine- 3-carboxamide | 561.3 |
| 312^{∧} | Ex. 113^{d} | 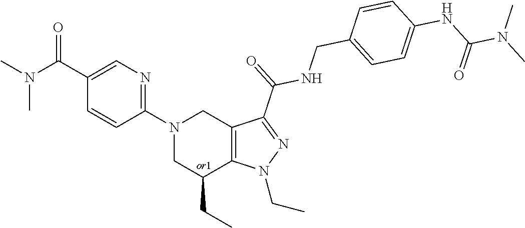 ¹H NMR (600 MHz, (CD₃)₂SO) δ 8.45 (t, 1H), 8.26-8.24 (m, 1H), 8.22 (s, 1H), 7.67- 7.63 (m, 1H), 7.40-7.36 (m, 2H), 7.20- 7.15 (m, 2H), 6.83 (d, 1H), 5.00 (d, 1H), 4.77 (d, 1H), 4.38-4.29 (m, 2H), 4.26- 4.19 (m, 1H), 4.16-4.03 (m, 2H), 3.20- 3.12 (m, 1H), 3.03-2.93 (m, 7H), 2.90 (s, 6H), 1.61-1.53 (m, 1H), 1.47-1.37 (m, 4H), 1.03 (t, 3H). | rel-(R or S)-5-(5- (dimethylcarbamoyl) pyridin-2-yl)-N-(4-(3,3- dimethylureido)benzyl)- 1,7-diethyl-4,5,6,7- tetrahydro-1H- pyrazolo[4,3-c]pyridine- 3-carboxamide | 547.3 |
| 313^{∧} | Ex. 113^{d} | 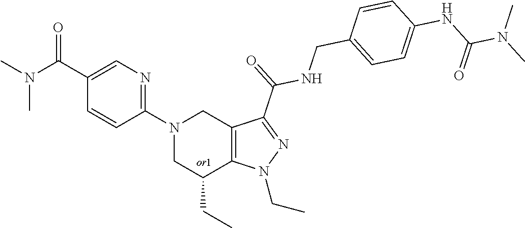 ¹H NMR (600 MHz, (CD₃)₂SO) δ 8.45 (t, 1H), 8.26-8.24 (m, 1H), 8.22 (s, 1H), 7.65 (dd, 1H), 7.40-7.36 (m, 2H), 7.19-7.14 (m, 2H), 6.83 (d, 1H), 5.00 (d, 1H), 4.77 (d, 1H), 4.38-4.28 (m, 2H), 4.26-4.19 (m, 1H), 4.15-4.04 (m, 2H), 3.20-3.14 (m, 1H), 3.03-2.94 (m, 7H), 2.90 (s, 6H), 1.62- 1.52 (m, 1H), 1.50-1.37 (m, 4H), 1.03 (t, 3H). | rel-(R or S)-5-(5- (dimethylcarbamoyl) pyridin-2-yl)-N-(4-(3,3- dimethylureido)benzyl)- 1,7-diethyl-4,5,6,7- tetrahydro-1H- pyrazolo[4,3-c]pyridine- 3-carboxamide | 547.3 |
| 314 | Ex. 127 | 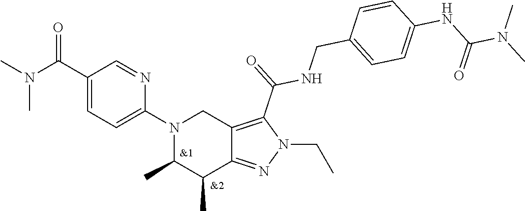 ¹H NMR (600 MHz, (CD₃)₂SO) δ 8.50- 8.46 (m, 1H), 8.28-8.20 (m, 2H), 7.71- 7.67 (m, 1H), 7.41-7.36 (m, 2H), 7.23- 7.16 (m, 2H), 6.92 (d, 1H), 5.07-4.92 (m, 2H), 4.41-4.28 (m, 2H), 4.17-4.04 (m, 3H), 3.10-3.03 (m, 1H), 3.02-2.94 (m, 6H), 2.90 (s, 6H), 1.39 (t, 3H), 1.18 (d, 3H), 1.00 (d, 3H). | rac-(6R,7S)-5-(5- (dimethylcarbamoyl) pyridin-2-yl)-N-(4-(3,3- dimethylureido)benzyl)- 2-ethyl-6,7-dimethyl- 4,5,6,7-tetrahydro-2H- pyrazolo[4,3-c]pyridine- 3-carboxamide | 547.2 |
| 315 | Ex. 567 | 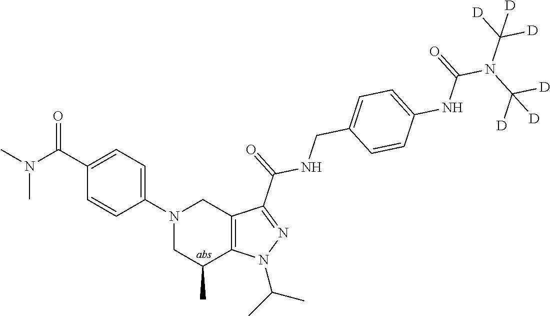 [SMILES: [2H]C([2H])([2H])N(C(=O)Nc1ccc(CNC(=O)c2nn(C(C)C)c3c2CN(c2ccc(C(=O)N(C)C)cc2)C[C@@H]3C)cc1)C([2H])([2H])[2H]] | (S)-N-(4-(3,3-bis(methyl- d₃)ureido)benzyl)-5-(4- (dimethylcarbamoyl) phenyl)-1-isopropyl-7- methyl-4,5,6,7- tetrahydro-1H- pyrazolo[4,3-c]pyridine- 3-carboxamide | 552.6 |
| 316^{∧} | Ex. 127 | 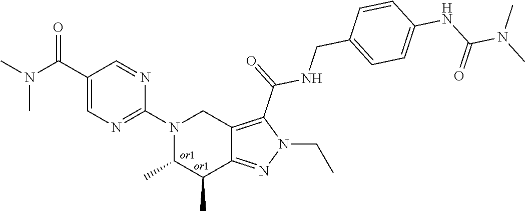 ¹H NMR (600 MHz, (CD₃)₂SO) δ 8.67- 8.62 (m, 1H), 8.29-8.25 (m, 2H), 7.72- 7.69 (m, 1H), 7.48-7.43 (m, 2H), 7.28- 7.20 (m, 2H), 6.81 (d, 1H), 5.13-4.96 (m, 2H), 4.45-4.40 (m, 2H), 4.37-4.30 (m, 1H), 4.29-4.19 (m, 2H), 3.10-3.04 (m, 1H), 2.98 (s, 6H), 2.93-2.89 (m, 6H), 1.32- 1.23 (m, 6H), 0.92-0.87 (m, 3H). | rel-(6S,7S)-5-(5- (dimethylcarbamoyl) pyridin-2-yl)-N-(4-(3,3- dimethylureido)benzyl)- 2-ethyl-6,7-dimethyl- 4,5,6,7-tetrahydro-2H- pyrazolo[4,3-c]pyridine- 3-carboxamide | 547.2 |
| 317^{∧} | Ex. 127 | 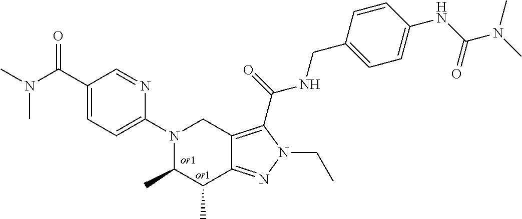 ¹H NMR (600 MHz, (CD₃)₂SO) δ 8.64 (t, 1H), 8.28-8.26 (m, 2H), 7.68 (dd, 1H), 7.49-7.44 (m, 2H), 7.27-7.22 (m, 2H), 6.77 (d, 1H), 5.10-4.97 (m, 2H), 4.42 (d, 2H), 4.38-4.30 (m, 1H), 4.29-4.17 (m, 2H), 3.11-3.03 (m, 1H), 2.98 (s, 6H), 2.94- 2.88 (m, 6H), 1.31-1.22 (m, 6H), 0.89 (d, 3H). | rel-(6R,7R)-5-(5- (dimethylcarbamoyl) pyridin-2-yl)-N-(4-(3,3- dimethylureido)benzyl)- 2-ethyl-6,7-dimethyl- 4,5,6,7-tetrahydro-2H- pyrazolo[4,3-c]pyridine- 3-carboxamide | 547.2 |
| 318^{∧} | Ex. 77 | 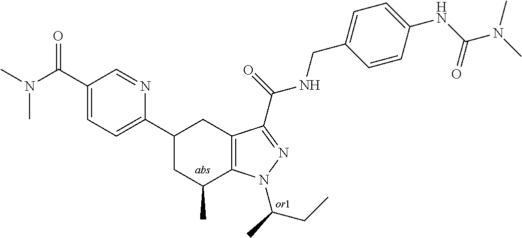 ¹H NMR (400 MHz, (CD₃)₂SO) δ 8.33 (t, 1H), 8.25-8.19 (m, 2H), 7.63 (dd, 1H), 7.41-7.34 (m, 2H), 7.19-7.14 (m, 2H), 6.88 (d, 1H), 5.09 (d, 1H), 4.49-4.16 (m, 5H), 3.25-3.14 (m, 2H), 2.97 (s, 6H), 2.89 (s, 6H), 1.91-1.64 (m, 2H), 1.41 (d, 3H), 1.17 (d, 3H), 0.66 (t, 3H). | (S)-1-((R*)-sec-butyl)-5- (5- (dimethylcarbamoyl) pyridin-2-yl)-N-(4-(3,3- dimethylureido)benzyl)- 7-methyl-4,5,6,7- tetrahydro-1H- pyrazolo[4,3-c]pyridine- 3-carboxamide | 561.3 |
| 319^{∧} | Ex. 77 | 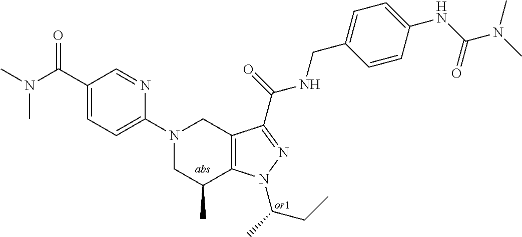 ¹H NMR (400 MHz, (CD₃)₂SO) δ 8.33 (t, 1H), 8.25-8.20 (m, 2H), 7.63 (dd, 1H), 7.41-7.34 (m, 2H), 7.20-7.13 (m, 2H), 6.88 (d, 1H), 5.10 (d, 1H), 4.50-4.17 (m, 5H), 3.25-3.14 (m, 2H), 2.97 (s, 6H), 2.89 (s, 6H), 2.05-1.70 (m, 2H), 1.35 (d, 3H), 1.23-1.16 (m, 3H), 0.77 (t, 3H). | (S)-1-((R*)-sec-butyl)-5- (5- (dimethylcarbamoyl) pyridin-2-yl)-N-(4-(3,3- dimethylureido)benzyl)- 7-methyl-4,5,6,7- tetrahydro-1H- pyrazolo[4,3-c]pyridine- 3-carboxamide | 561.3 |
| 320 | Ex. 42^{g} | 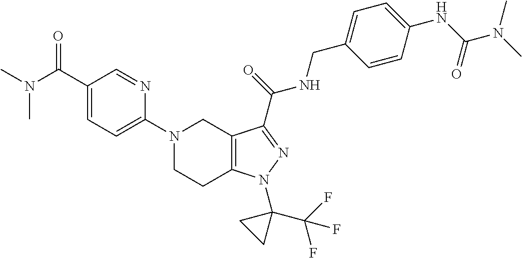 ¹H NMR (400 MHz, CD₃OD) δ 8.54 (t, 1H), 8.30 (d, 1H), 7.71 (dd, 1H), 7.39-7.33 (m, 2H), 7.33-7.27 (m, 2H), 6.93 (d, 1H), 4.81- 4.75 (m, 2H), 4.54-4.50 (m, 2H), 4.10 (t, 2H), 3.11 (s, 6H), 3.03 (s, 6H), 2.97 (t, 2H), 1.72-1.62 (m, 4H). | 5-(5- (dimethylcarbamoyl) pyridin-2-yl)-N-(4-(3,3- dimethylureido)benzyl)- 1-(1- (trifluoromethyl)cyclopropyl)- 4,5,6,7-tetrahydro- 1H-pyrazolo[4,3- c]pyridine-3- carboxamide | 599.3 |
| 321 | Ex. 113 | 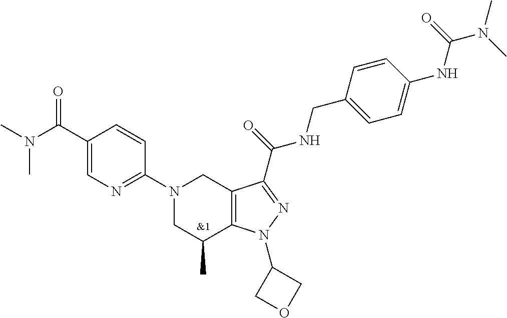 ¹H NMR (400 MHz, CDCl₃) δ 8.34-8.28 (m, 1H), 7.63 (dd, 1H), 7.40-7.33 (m, 2H), 7.32-7.27 (m, 3H), 6.77 (d, 1H), 6.33 (s, 1H), 5.44-5.33 (m, 1H), 5.25 (t, 1H), 5.08 (t, 1H), 5.02-4.90 (m, 3H), 4.71-4.63 (m, 1H), 4.62-4.51 (m, 2H), 4.50-4.40 (m, 1H), 3.44-3.37 (m, 1H), 3.08 (s, 6H), 3.04- 2.99 (m, 7H), 1.21 (d, 3H). | rac-(R)-5-(5- (dimethylcarbamoyl) pyridin-2-yl)-N-(4-(3,3- dimethylureido)benzyl)- 7-methyl-1-(oxetan-3- yl)-4,5,6,7-tetrahydro- 1H-pyrazolo[4,3- c]pyridine-3- carboxamide | 561.3 |
| 322 | Ex. 322 | 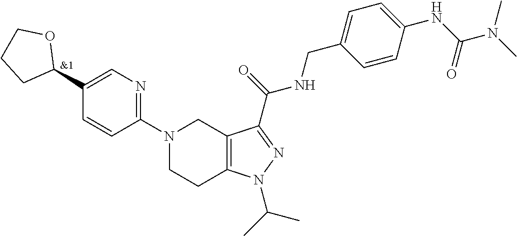 [SMILES: CC(C)n1nc(C(=O)NCc2ccc(NC(=O)N(C)C)cc2)c2c1CCN(c1ccc([C@H]3CCCO3)cn1)C2] ¹H NMR (400 MHz, CD₃OD) δ 8.10 (d, 1H), 7.61 (dd, 1H), 7.39-7.35 (m, 2H), 7.34- 7.28 (m, 2H), 6.91 (d, 1H), 4.81-4.76 (m, 1H), 4.70 (s, 2H), 4.56-4.50 (m, 3H), 4.12- 3.87 (m, 4H), 3.05-3.03 (m, 6H), 2.87 (t, 2H), 2.36-2.24 (m, 1H), 2.15-2.00 (m, 2H), 1.90-1.77 (m, 1H), 1.49 (d, 6H). | rac-(R)-N-(4-(3,3- dimethylureido)benzyl)- 1-isopropyl-5-(5- (tetrahydrofuran-2- yl)pyridin-2-yl)-4,5,6,7- tetrahydro-1H- pyrazolo[4,3-c]pyridine- 3-carboxamide | 532.2 |
| 323 | Ex. 126 | 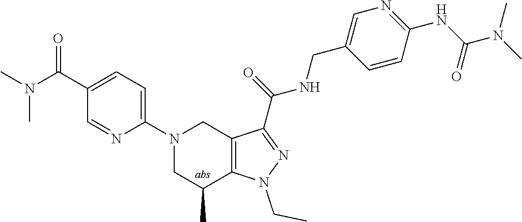 | (S)-5-(5- (dimethylcarbamoyl) pyridin-2-yl)-N-((6-(3,3- dimethylureido)pyridin- 3-yl)methyl)-1-ethyl-7- methyl-4,5,6,7- tetrahydro-1H- pyrazolo[4,3-c]pyridine- 3-carboxamide | 534.5 |
| 324 | Ex. 125 | 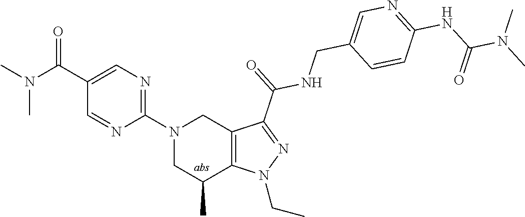 ¹H NMR* (600 MHz, (CD₃)₂SO) δ 9.66 (s, 1H), 8.72-8.66 (m, 1H), 8.51 (s, 2H), 8.24- 8.21 (m, 1H), 8.00-7.89 (s, 1H), 7.69 (d, 1H), 5.40 (d, 1H), 4.68 (dd, 1H), 4.43-4.31 (m, 3H), 4.15-4.05 (m, 2H), 3.47 (dd, 1H), 3.28-3.22 (m, 1H), 3.18-2.95 (m, 12H), 1.39 (t, 3H), 1.16 (d, 3H). | (S)-5-(5- (dimethylcarbamoyl) pyrimidin-2-yl)-N-((6-(3,3- dimethylureido)pyridin- 3-yl)methyl)-1-ethyl-7- methyl-4,5,6,7- tetrahydro-1H- pyrazolo[4,3-c]pyridine- 3-carboxamide | 535.4 |
| 325 | Ex. 193 | 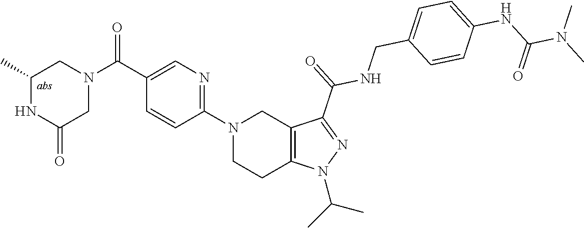 | (R)-N-(4-(3,3- dimethylureido)benzyl)- 1-isopropyl-5-(5-(3- methyl-5-oxopiperazine- 1-carbonyl)pyridin-2-yl)- 4,5,6,7-tetrahydro-1H- pyrazolo[4,3-c]pyridine- 3-carboxamide | 602.5 |
| 326 | Ex. 193 | 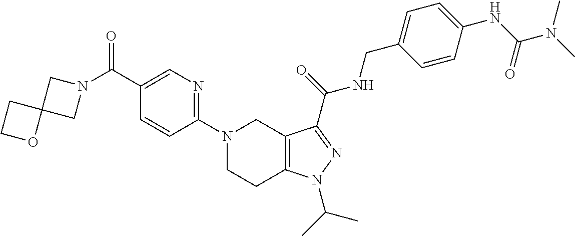 [SMILES: CC(C)n1nc(C(=O)NCc2ccc(NC(=O)N(C)C)cc2)c2c1CCN(c1ccc(C(=O)N3CC4(CCO4)C3)cn1)C2] | 5-(5-(1-oxa-6- azaspiro[3.3]heptane-6- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 587.5 |
| 327 | Ex. 193 | 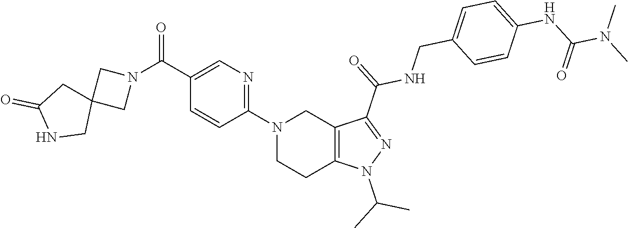 [SMILES: CC(C)n1nc(C(=O)NCc2ccc(NC(=O)N(C)C)cc2)c2c1CCN(c1ccc(C(=O)N3CC4(CNC(=O)C4)C3)cn1)C2] | N-(4-(3,3- dimethylureido)benzyl)- 1-isopropyl-5-(5-(7-oxo- 2,6- diazaspiro[3.4]octane-2- carbonyl)pyridin-2-yl)- 4,5,6,7-tetrahydro-1H- pyrazolo[4,3-c]pyridine- 3-carboxamide | 614.5 |
| 328 | Ex. 193 | 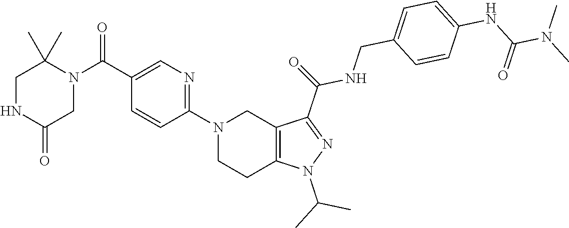 [SMILES: CC(C)n1nc(C(=O)NCc2ccc(NC(=O)N(C)C)cc2)c2c1CCN(c1ccc(C(=O)N3CC(=O)NCC3(C)C)cn1)C2] | 5-(5-(2,2-dimethyl-5- oxopiperazine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 616.6 |
| 329 | Ex. 193 | 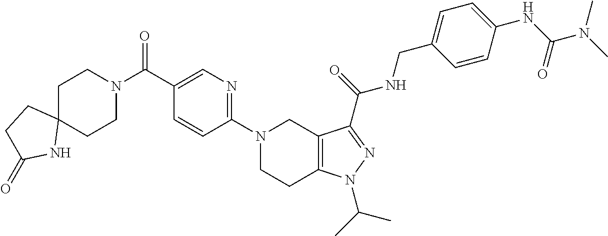 [SMILES: CC(C)n1nc(C(=O)NCc2ccc(NC(=O)N(C)C)cc2)c2c1CCN(c1ccc(C(=O)N3CCC4(CCC(=O)N4)CC3)cn1)C2] | N-(4-(3,3- dimethylureido)benzyl)- 1-isopropyl-5-(5-(2-oxo- 1,8- diazaspiro[4.5]decane- 8-carbonyl)pyridin-2-yl)- 4,5,6,7-tetrahydro-1H- pyrazolo[4,3-c]pyridine- 3-carboxamide | 642.6 |
| 330 | Ex. 193 | 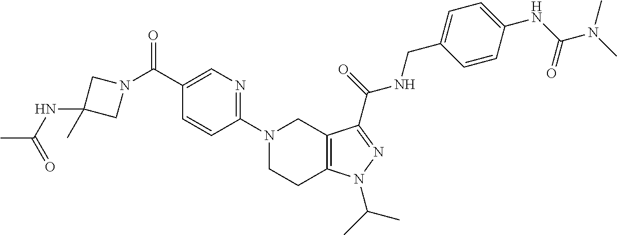 [SMILES: CC(=O)NC1(C)CN(C(=O)c2ccc(N3CCc4c(c(C(=O)NCc5ccc(NC(=O)N(C)C)cc5)nn4C(C)C)C3)nc2)C1] | 5-(5-(3-acetamido-3- methylazetidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 616.5 |
| 331 | Ex. 193 | 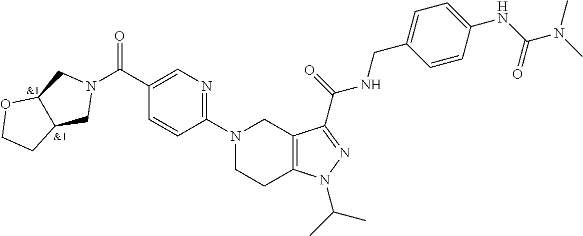 [SMILES: CC(C)n1nc(C(=O)NCc2ccc(NC(=O)N(C)C)cc2)c2c1CCN(c1ccc(C(=O)N3C[C@H]4CCO[C@H]4C3)cn1)C2] | rac-N-(4-(3,3- dimethylureido)benzyl)- 5-(5-((3aR,6aR)- hexahydro-2H-furo[2,3- c]pyrrole-5- carbonyl)pyridin-2-yl)-1- isopropyl-4,5,6,7- tetrahydro-1H- pyrazolo[4,3-c]pyridine- 3-carboxamide | 601.5 |
| 332 | Ex. 193 | 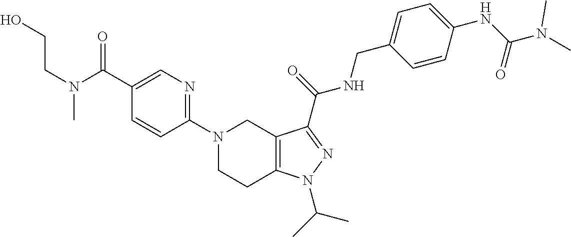 [SMILES: CC(C)n1nc(C(=O)NCc2ccc(NC(=O)N(C)C)cc2)c2c1CCN(c1ccc(C(=O)N(C)CCO)cn1)C2] | N-(4-(3,3- dimethylureido)benzyl)- 5-(5-((2- hydroxyethyl)(methyl) carbamoyl)pyridin-2-yl)-1- isopropyl-4,5,6,7- tetrahydro-1H- pyrazolo[4,3-c]pyridine- 3-carboxamide | 563.5 |
| 333 | Ex. 193 | 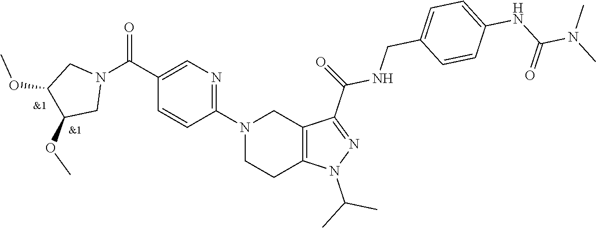 | rac-5-(5-((3R,4R)-3,4- dimethoxypyrrolidine-1- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 619.5 |
| 334 | Ex 193 | 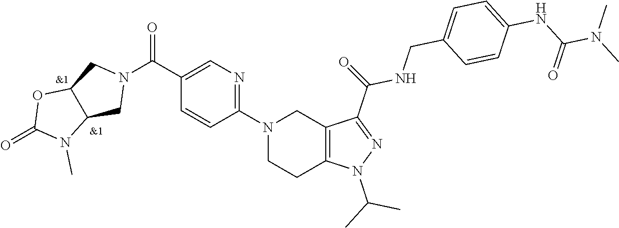 [SMILES: CC(C)n1nc(C(=O)NCc2ccc(NC(=O)N(C)C)cc2)c2c1CCN(c1ccc(C(=O)N3C[C@@H]4OC(=O)N(C)[C@@H]4C3)cn1)C2] | rac-N-(4-(3,3- dimethylureido)benzyl)- 1-isopropyl-5-(5- ((3aR,6aS)-3-methyl-2- oxohexahydro-2H- pyrrolo[3,4-d]oxazole-5- carbonyl)pyridin-2-yl)- 4,5,6,7-tetrahydro-1H- pyrazolo[4,3-c]pyridine- 3-carboxamide | 630.6 |
| 335^{∧} | Ex. 127 | 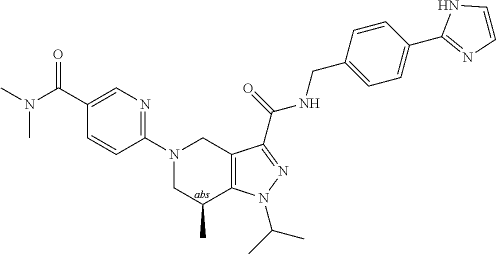 [SMILES: CC(C)n1nc(C(=O)NCc2ccc(-c3ncc[nH]3)cc2)c2c1[C@@H](C)CN(c1ccc(C(=O)N(C)C)cn1)C2] ¹H NMR (400 MHz, (CD₃)₂SO) δ 12.42 (s, 1H), 8.51-8.45 (m, 3H), 7.86-7.82 (m, 2H), 7.37-7.32 (m, 2H), 7.19 (s, 1H), 6.96 (s, 1H), 5.44 (d, 1H), 4.75-4.69 (m, 1H), 4.55-4.23 (m, 4H), 3.42-3.36 (m, 2H), 2.96 (s, 6H), 1.44-1.36 (m, 6H), 1.12 (d, 3H). | (S)-N-(4-(1H-imidazol-2- yl)benzyl)-5-(5- (dimethylcarbamoyl) pyrimidin-2-yl)-1-isopropyl- 7-methyl-4,5,6,7- tetrahydro-1H- pyrazolo[4,3-c]pyridine- 3-carboxamide | 528.3 |
| 336^{∧} | Ex. 23^{g} | 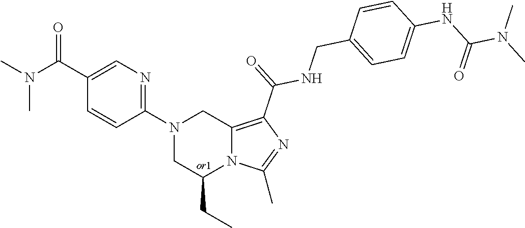 ¹H NMR (400 MHz, CD₃OD) δ 8.31 (d, 1H), 7.73 (dd, 1H), 7.35-7.30 (m, 2H), 7.28- 7.23 (m, 2H), 6.88 (d, 1H), 5.27-5.10 (m, 2H), 4.68-4.60 (m, 2H), 4.48 (s, 2H), 4.35- 4.28 (m, 1H), 3.09 (s, 6H), 2.99 (s, 6H), 2.39 (s, 3H), 1.76-1.65 (m, 2H), 1.06 (t, 3H). | rel-(R or S)-7-(5- (dimethylcarbamoyl) pyridin-2-yl)-N-(4-(3,3- dimethylureido)benzyl)- 5-ethyl-3-methyl-5,6,7,8- tetrahydroimidazo[1,5- a]pyrazine-1- carboxamide | 533.3 |
| 337 | Ex. 286 | 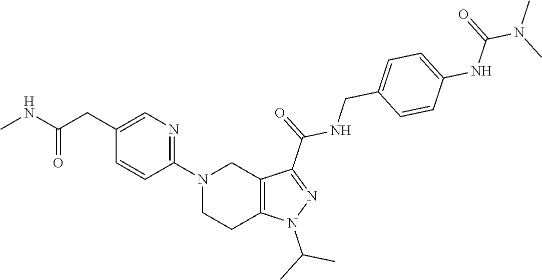 ¹H NMR (400 MHz, (CD₃)₂SO) δ 8.29 (t, 1H), 8.21 (s, 1H), 7.96-7.94 (m, 1H), 7.88- 7.82 (m, 1H), 7.45-7.34 (m, 3H), 7.15 (d, 2H), 6.80 (d, 1H), 4.61 (s, 2H), 4.50-4.41 (m, 1H), 4.32 (d, 2H), 3.87-3.80 (m, 2H), 3.22 (s, 2H), 2.89 (s, 6H), 2.81-2.73 (m, 2H), 2.53 (d, 3H), 1.36 (d, 6H). | N-(4-(3,3- dimethylureido)benzyl)- 1-isopropyl-5-(5-(2- (methylamino)-2- oxoethyl)pyridin-2-yl)- 4,5,6,7-tetrahydro-1H- pyrazolo[4,3-c]pyridine- 3-carboxamide | 533.3 |
| 338 | Ex. 338 | 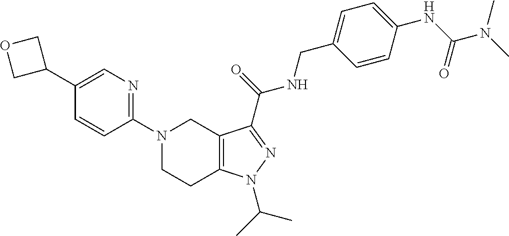 [SMILES: CC(C)n1nc(C(=O)NCc2ccc(NC(=O)N(C)C)cc2)c2c1CCN(c1ccc(C3COC3)cn1)C2] ¹H NMR (400 MHz, CD₃OD) δ 8.06 (d, 1H), 7.79 (dd, 1H), 7.36-7.31 (m, 2H), 7.30- 7.25 (m, 2H), 6.94 (d, 1H), 5.08-5.01 (m, 2H), 4.72-4.65 (m, 4H), 4.55-4.47 (m, 3H), 4.23-4.14 (m, 1H), 3.97 (t, 2H), 3.00 (s, 6H), 2.84 (t, 2H), 1.45 (d, 6H). | N-(4-(3,3- dimethylureido)benzyl)- 1-isopropyl-5-(5- (oxetan-3-yl)pyridin-2- yl)-4,5,6,7-tetrahydro- 1H-pyrazolo[4,3- c]pyridine-3- carboxamide | 518.3 |
| 339 | Ex. 322 | 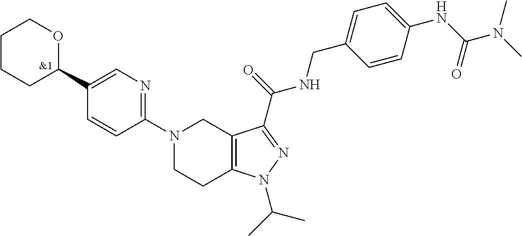 [SMILES: CC(C)n1nc(C(=O)NCc2ccc(NC(=O)N(C)C)cc2)c2c1CCN(c1ccc([C@H]3CCCCO3)cn1)C2] 1H NMR (400 MHz, CD₃OD) δ 8.08 (d, 1H), 7.59 (dd, 1H), 7.36-7.32 (m, 2H), 7.31- 7.26 (m, 2H), 6.87 (d, 1H), 4.67 (s, 2H), 4.56-4.47 (m, 3H), 4.32-4.26 (m, 1H), 4.08-4.01 (m, 1H), 3.98 (t, 2H), 3.67- 3.58 (m, 1H), 3.01 (s, 6H), 2.84 (t, 2H), 2.00- 1.91 (m, 1H), 1.81-1.56 (m, 5H), 1.46 (d, 6H). | rac-(R)-N-(4-(3,3- dimethylureido)benzyl)- 1-isopropyl-5-(5- (tetrahydro-2H-pyran-2- yl)pyridin-2-yl)-4,5,6,7- tetrahydro-1H- pyrazolo[4,3-c]pyridine- 3-carboxamide | 546.3 |
| 340 | Ex. 127 | 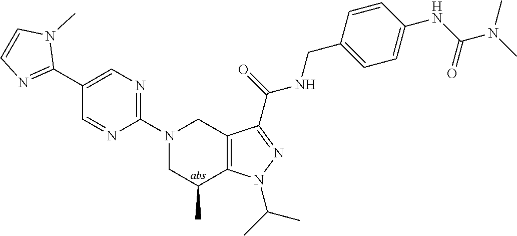 [SMILES: CC(C)n1nc(C(=O)NCc2ccc(NC(=O)N(C)C)cc2)c2c1[C@@H](C)CN(c1ncc(-c3nccn3C)cn1)C2] ¹H NMR (400 MHz, (CD₃)₂SO) δ 8.50 (s, 2H), 8.32 (t, 1H), 8.21 (s, 1H), 7.68 (s, 1H), 7.39-7.34 (m, 2H), 7.18-7.12 (m, 2H), 7.01 (d, 1H), 5.41 (d, 1H), 4.75-4.68 (m, 1H), 4.55-4.46 (m, 1H), 4.39-4.25 (m, 3H), 3.61 (s, 3H), 3.27-3.20 (m, 2H), 2.88 (s, 6H), 1.43-1.35 (m, 6H), 1.15 (d, 3H). | (S)-N-(4-(3,3- dimethylureido)benzyl)- 1-isopropyl-7-methyl-5- (5-(1-methyl-1H- imidazol-2-yl)pyrimidin- 2-yl)-4,5,6,7-tetrahydro- 1H-pyrazolo[4,3- c]pyridine-3- carboxamide | 557.6 |
| 341 | Ex. 127 | 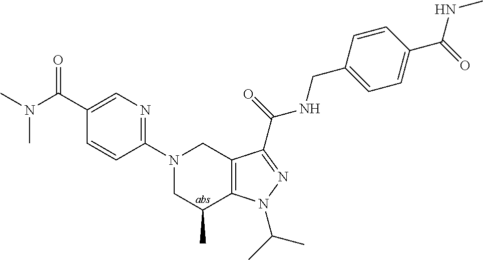 ¹H NMR (400 MHz, (CD₃)₂SO) δ 8.53 (t, 1H), 8.38-8.32 (m, 1H), 8.21 (d, 1H), 7.78- 7.71 (m, 2H), 7.61 (dd, 1H), 7.38-7.31 (m, 2H), 6.86 (d, 1H), 5.07 (d, 1H), 4.58- 4.33 (m, 4H), 4.27-4.12 (m, 1H), 3.28- 3.16 (m, 2H), 2.95 (s, 6H), 2.75 (d, 3H), 1.44-1.34 (m, 6H), 1.18 (d, 3H). | (S)-5-(5- (dimethylcarbamoyl) pyridin-2-yl)-1-isopropyl-7- methyl-N-(4- (methylcarbamoyl)benzyl)- 4,5,6,7-tetrahydro-1H- pyrazolo[4,3-c]pyridine- 3-carboxamide | 518.2 |
| 342 | Ex. 127 | 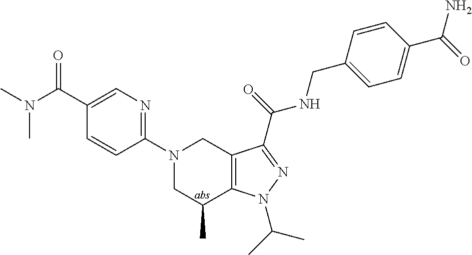 [SMILES: CC(C)n1nc(C(=O)NCc2ccc(C(N)=O)cc2)c2c1[C@@H](C)CN(c1ccc(C(=O)N(C)C)cn1)C2] ¹H NMR (400 MHz, (CD₃)₂SO) δ 8.55 (t, 1H), 8.24 (d, 1H), 7.94-7.88 (m, 1H), 7.85- 7.79 (m, 2H), 7.64 (dd, 1H), 7.40-7.35 (m, 2H), 7.32-7.28 (m, 1H), 6.89 (d, 1H), 5.09 (d, 1H), 4.60-4.37 (m, 4H), 4.23 (d, 1H), 3.27-3.19 (m, 2H), 2.98 (s, 6H), 1.48- 1.39 (m, 6H), 1.20 (d, 3H). | (S)-N-(4- carbamoylbenzyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 504.1 |
| 343 | Ex. 127 | 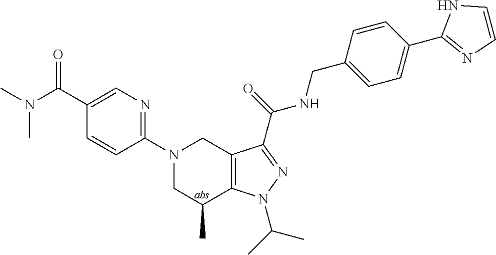 [SMILES: CC(C)n1nc(C(=O)NCc2ccc(-c3ncc[nH]3)cc2)c2c1[C@@H](C)CN(c1ccc(C(=O)N(C)C)cn1)C2] ¹H NMR (400 MHz, (CD₃)₂SO) δ 12.43 (s, 1H), 8.49 (t, 1H), 8.22 (d, 1H), 7.88-7.80 (m, 2H), 7.61 (dd, 1H), 7.38-7.31 (m, 2H), 7.20 (s, 1H), 6.97 (s, 1H), 6.87 (d, 1H), 5.08 (d, 1H), 4.59-4.35 (m, 4H), 4.22 (d, 1H), 3.25-3.16 (m, 2H), 2.95 (s, 6H), 1.48- 1.33 (m, 6H), 1.18 (d, 3H). | (S)-N-(4-(1H-imidazol-2- yl)benzyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 527.3 |
| 344 | Ex. 113 | 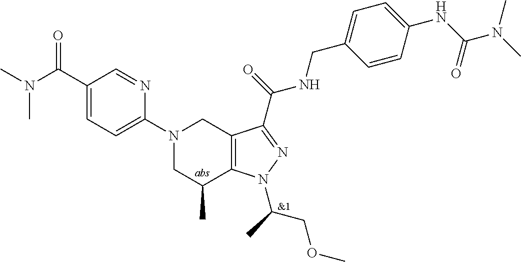 ¹H NMR (400 MHz, (CD₃)₂SO) δ 8.35 (t, 1H), 8.22-8.18 (m, 2H), 7.60 (dd, 1H), 7.39-7.32 (m, 2H), 7.17-7.11 (m, 2H), 6.85 (d, 1H), 5.15-5.01 (m, 1H), 4.53- 4.12 (m, 5H), 3.76-3.49 (m, 2H), 3.25- 3.15 (m, 2H), 3.13 (s, 3H), 2.94 (s, 6H), 2.87 (s, 6H), 1.37-1.27 (m, 3H), 1.21- 1.13 (m, 3H). | (S)-5-(5- (dimethylcarbamoyl) pyridin-2-yl)-N-(4-(3,3- dimethylureido)benzyl)- 1-((RS)-1- methoxypropan-2-yl)-7- methyl-4,5,6,7- tetrahydro-1H- pyrazolo[4,3-c]pyridine- 3-carboxamide | 577.3 |
| 345 | Ex. 322 | 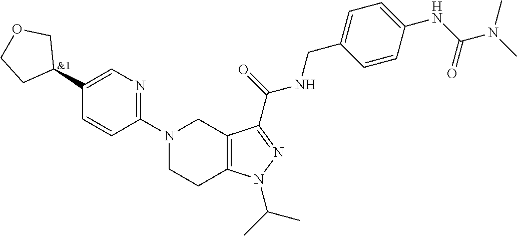 [SMILES: CC(C)n1nc(C(=O)NCc2ccc(NC(=O)N(C)C)cc2)c2c1CCN(c1ccc([C@H]3CCOC3)cn1)C2] ¹H NMR (400 MHz, CD₃OD) δ 8.34-8.28 (m, 1H), 8.00 (d, 1H), 7.52 (dd, 1H), 7.34- 7.30 (m, 2H), 7.28-7.24 (m, 2H), 6.86 (d, 1H), 4.64-4.62 (m, 2H), 4.53-4.45 (m, 3H), 4.07-4.00 (m, 2H), 3.96-3.84 (m, 3H), 3.63-3.57 (m, 1H), 3.37-3.32 (m, 1H), 2.99 (s, 6H), 2.82 (t, 2H), 2.37-2.27 (m, 1H), 2.00-1.87 (m, 1H), 1.44 (d, 6H). | rac-(R)-N-(4-(3,3- dimethylureido)benzyl)- 1-isopropyl-5-(5- (tetrahydrofuran-3- yl)pyridin-2-yl)-4,5,6,7- tetrahydro-1H- pyrazolo[4,3-c]pyridine- 3-carboxamide | 532.3 |
| 346 | Ex. 127 | 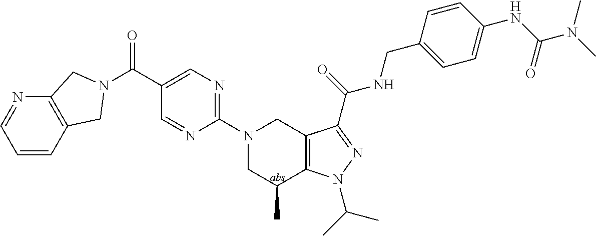 [SMILES: CC(C)n1nc(C(=O)NCc2ccc(NC(=O)N(C)C)cc2)c2c1[C@@H](C)CN(c1ncc(C(=O)N3Cc4cccnc4C3)cn1)C2] ¹H NMR (400 MHz, (CD₃)₂SO) δ 8.72 (d, 2H), 8.47-8.41 (m, 1H), 8.34 (t, 1H), 8.21 (s, 1H), 7.84-7.67 (m, 1H), 7.39-7.33 (m, 2H), 7.32-7.25 (m, 1H), 7.19-7.11 (m, 2H), 5.48 (d, 1H), 5.04-4.71 (m, 5H), 4.55- 4.44 (m, 1H), 4.38-4.24 (m, 3H), 3.50- 3.38 (m, 1H), 3.28-3.20 (m, 1H), 2.88 (s, 6H), 1.44-1.35 (m, 6H), 1.14 (d, 3H). | (S)-5-(5-(6,7-dihydro- 5H-pyrrolo[3,4- b]pyridine-6- carbonyl)pyrimidin-2-yl)- N-(4-(3,3- dimethylureido)benzyl)- 1-isopropyl-7-methyl- 4,5,6,7-tetrahydro-1H- pyrazolo[4,3-c]pyridine- 3-carboxamide | 623.3 |
| 347^{∧} | Ex. 77^{e} | 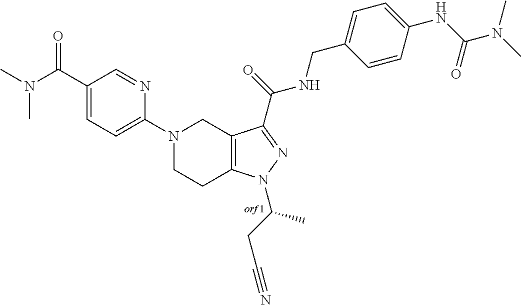 ¹H NMR (400 MHz, (CD₃)₂SO) δ 8.37 (t, 1H), 8.27-8.22 (m, 2H), 7.66 (dd, 1H), 7.44-7.39 (m, 2H), 7.22-7.16 (m, 2H), 6.92 (d, 1H), 4.82-4.70 (m, 3H), 4.38 (d, 2H), 3.98 (t, 2H), 3.08 (d, 2H), 2.99 (s, 6H), 2.94-2.85 (m, 8H), 1.49 (d, 3H). | rel-(R or S)-1-(1- cyanopropan-2-yl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 558.1 |
| 348^{∧} | Ex. 77^{e} | 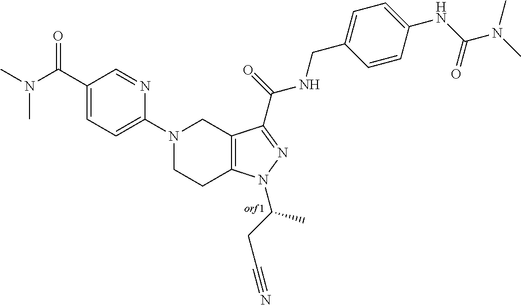 ¹H NMR (400 MHz, (CD₃)₂SO) δ 8.37 (t, 1H), 8.27-8.21 (m, 2H), 7.66 (dd, 1H), 7.45-7.37 (m, 2H), 7.22-7.14 (m, 2H), 6.92 (d, 1H), 4.83-4.70 (m, 3H), 4.38 (d, 2H), 3.98 (t, 2H), 3.07 (d, 2H), 2.99 (s, 6H), 2.94-2.86 (m, 8H), 1.49 (d, 3H). | rel-(R or S)-1-(1- cyanopropan-2-yl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 558.1 |
| 349 | Ex. 127 | 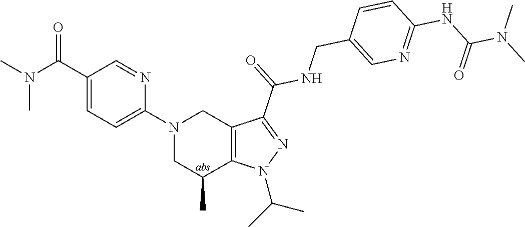 ¹H NMR* (600 MHz, (CD₃)₂SO) δ 9.88- 9.70 (m, 1H), 8.63-8.57 (m, 1H), 8.27- 8.20 (m, 2H), 8.06-7.99 (m, 1H), 7.71- 7.62 (m, 2H), 6.94-6.88 (m, 1H), 5.09 (d, 1H), 4.59-4.50 (m, 1H), 4.45-4.33 (m, 3H), 4.25-4.19 (m, 1H), 3.37-3.32 (m, 1H), 3.28-3.21 (m, 1H), 3.00-2.94 (m, 12H), 1.45-1.40 (m, 6H), 1.21-1.18 (m, 3H). | (S)-5-(5- (dimethylcarbamoyl) pyridin-2-yl)-N-((6-(3,3- dimethylureido)pyridin- 3-yl)methyl)-1-isopropyl- 7-methyl-4,5,6,7- tetrahydro-1H- pyrazolo[4,3-c]pyridine- 3-carboxamide | 548.5 |
| 350 | Ex. 127 | 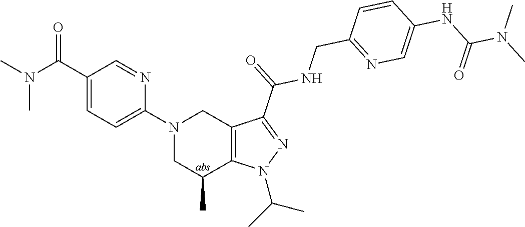 ¹H NMR* (600 MHz, (CD₃)₂SO) δ 8.96- 8.86 (m, 1H), 8.85-8.76 (m, 1H), 8.67- 8.59 (m, 1H), 8.25-8.17 (m, 2H), 7.69- 7.55 (m, 2H), 6.95-6.88 (m, 1H), 5.09 (d, 1H), 4.66-4.50 (m, 3H), 4.43-4.40 (m, 1H), 4.22 (d, 1H), 3.37-3.34 (m, 1H), 3.29- 3.24 (m, 1H), 3.00-2.97 (m, 6H), 2.96- 2.93 (m, 6H), 1.47-1.42 (m, 6H), 1.22- 1.19 (m, 3H). | (S)-5-(5- (dimethylcarbamoyl) pyridin-2-yl)-N-((5-(3,3- dimethylureido)pyridin- 2-yl)methyl)-1-isopropyl- 7-methyl-4,5,6,7- tetrahydro-1H- pyrazolo[4,3-c]pyridine- 3-carboxamide | 548.5 |
| 351 | Ex. 113^{m} | 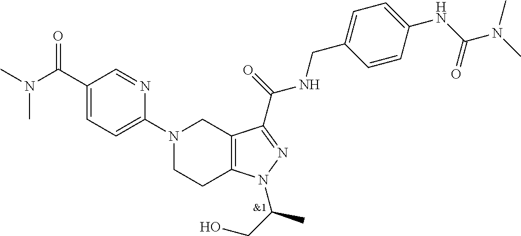 ¹H NMR (400 MHz, (CD₃)₂SO) δ 8.35 (t, 1H), 8.26-8.20 (m, 2H), 7.64 (dd, 1H), 7.43-7.36 (m, 2H), 7.20-7.14 (m, 2H), 6.89 (d, 1H), 4.90-4.69 (m, 3H), 4.39- 4.21 (m, 3H), 4.00-3.86 (m, 2H), 3.66- 3.56 (m, 2H), 2.98 (s, 6H), 2.91 (s, 6H), 2.86-2.80 (m, 2H), 1.37 (d, 3H). | rac-(R)-5-(5- (dimethylcarbamoyl) pyridin-2-yl)-N-(4-(3,3- dimethylureido)benzyl)- 1-(1-hydroxypropan-2- yl)-4,5,6,7-tetrahydro- 1H-pyrazolo[4,3- c]pyridine-3- carboxamide | 549.3 |
| 352^{∧} | Ex. 127 | 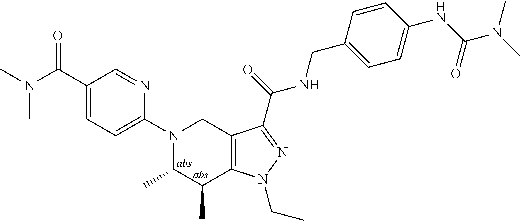 ¹H NMR (600 MHz, (CD₃)₂SO) δ 8.47 (t, 1H), 8.27 (d, 1H), 8.22 (s, 1H), 7.66 (dd, 1H), 7.41-7.36 (m, 2H), 7.20-7.16 (m, 2H), 6.86 (d, 1H), 5.09-4.92 (m, 2H), 4.40- 4.26 (m, 2H), 4.15-4.04 (m, 3H), 3.09- 3.02 (m, 1H), 3.01-2.94 (m, 6H), 2.90 (s, 6H), 1.38 (t, 3H), 1.18 (d, 3H), 0.99 (d, 3H). | (6S,7S)-5-(5- (dimethylcarbamoyl) pyridin-2-yl)-N-(4-(3,3- dimethylureido)benzyl)- 1-ethyl-6,7-dimethyl- 4,5,6,7-tetrahydro-1H- pyrazolo[4,3-c]pyridine- 3-carboxamide | 547.2 |
| 353^{∧} | Ex. 127 | 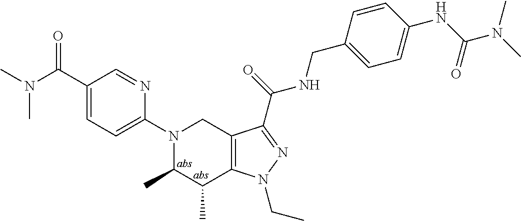 ¹H NMR (600 MHz, (CD₃)₂SO) δ 8.47 (t, 1H), 8.27 (d, 1H), 8.22 (s, 1H), 7.66 (dd, 1H), 7.40-7.36 (m, 2H), 7.20-7.16 (m, 2H), 6.86 (d, 1H), 5.07-4.92 (m, 2H), 4.40- 4.27 (m, 2H), 4.16-4.04 (m, 3H), 3.08- 3.02 (m, 1H), 3.01-2.94 (m, 6H), 2.90 (s, 6H), 1.38 (t, 3H), 1.18 (d, 3H), 0.99 (d, 3H). | (6R,7R)-5-(5- (dimethylcarbamoyl) pyridin-2-yl)-N-(4-(3,3- dimethylureido)benzyl)- 1-ethyl-6,7-dimethyl- 4,5,6,7-tetrahydro-1H- pyrazolo[4,3-c]pyridine- 3-carboxamide | 547.3 |
| 354^{∧} | Ex. 127 | 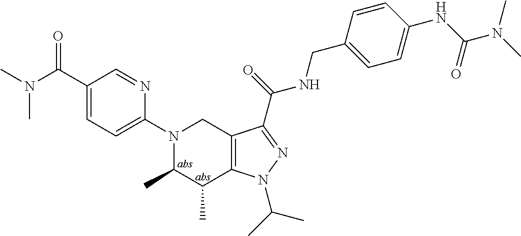 ¹H NMR (600 MHz, (CD₃)₂SO) δ 8.36 (t, 1H), 8.27 (d, 1H), 8.23 (s, 1H), 7.66 (dd, 1H), 7.42-7.36 (m, 2H), 7.21-7.17 (m, 2H), 6.86 (d, 1H), 5.05-4.94 (m, 2H), 4.58- 4.48 (m, 1H), 4.40-4.28 (m, 2H), 4.10 (d, 1H), 3.09-3.02 (m, 1H), 3.01-2.93 (m, 6H), 2.91 (s, 6H), 1.45 (d, 3H), 1.40 (d, 3H), 1.17 (d, 3H), 0.99 (d, 3H). | (6R,7R)-5-(5- (dimethylcarbamoyl) pyridin-2-yl)-N-(4-(3,3- dimethylureido)benzyl)- 1-isopropyl-6,7- dimethyl-4,5,6,7- tetrahydro-1H- pyrazolo[4,3-c]pyridine- 3-carboxamide | 561.3 |
| 355^{∧} | Ex. 127 | 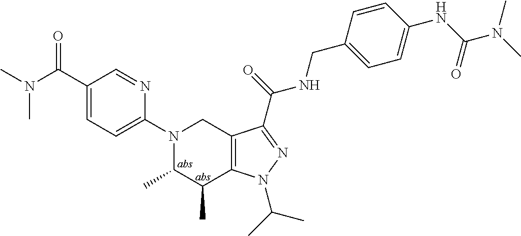 ¹H NMR (600 MHz, (CD₃)₂SO) δ 8.36 (t, 1H), 8.27 (d, 1H), 8.23 (s, 1H), 7.66 (dd, 1H), 7.42-7.37 (m, 2H), 7.21-7.17 (m, 2H), 6.86 (d, 1H), 5.05-4.94 (m, 2H), 4.58- 4.48 (m, 1H), 4.40-4.28 (m, 2H), 4.10 (d, 1H), 3.09-3.03 (m, 1H), 3.02-2.94 (m, 6H), 2.91 (s, 6H), 1.45 (d, 3H), 1.40 (d, 3H), 1.17 (d, 3H), 0.99 (d, 3H). | (6S,7S)-5-(5- (dimethylcarbamoyl) pyridin-2-yl)-N-(4-(3,3- dimethylureido)benzyl)- 1-isopropyl-6,7- dimethyl-4,5,6,7- tetrahydro-1H- pyrazolo[4,3-c]pyridine- 3-carboxamide | 561.3 |
| 356^{∧} | Ex. 546 | 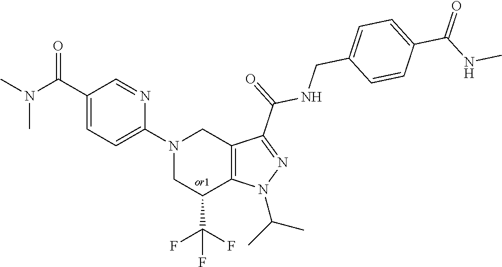 ¹H NMR (600 MHz, (CD₃)₂SO) δ 8.72 (t, 1H), 8.37 (q, 1H), 8.27 (d, 1H), 7.78 (d, 2H), 7.68 (dd, 1H), 7.40 (d, 2H), 6.83 (d, 1H), 5.27 (d, 1H), 5.06 (d, 1H), 4.72-4.64 (m, 1H), 4.56-4.43 (m, 3H), 4.27 (d, 1H), 3.31- 3.27 (m, 1H), 2.98 (s, 6H), 2.77 (d, 3H), 1.44 (dd, 6H). | rel-(R or S)-5-(5- (dimethylcarbamoyl) pyridin-2-yl)-1-isopropyl-N- (4- (methylcarbamoyl)benzyl)- 7-(trifluoromethyl)- 4,5,6,7-tetrahydro-1H- pyrazolo[4,3-c]pyridine- 3-carboxamide | 572.2 |
| 357^{∧} | Ex. 546 | 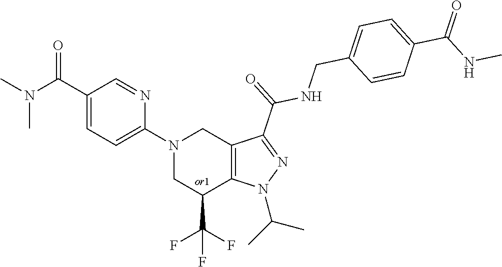 ¹H NMR (600 MHz, (CD₃)₂SO) δ 8.75- 8.70 (m, 1H), 8.42-8.34 (m, 1H), 8.28- 8.25 (m, 1H), 7.81-7.76 (m, 2H), 7.71- 7.66 (m, 1H), 7.42-7.37 (m, 2H), 6.83 (d, 1H), 5.28 (d, 1H), 5.06 (d, 1H), 4.74-4.64 (m, 1H), 4.56-4.43 (m, 3H), 4.27 (d, 1H), 3.31-3.27 (m, 1H), 2.98 (s, 6H), 2.78- 2.75 (m, 3H), 1.44 (dd, 6H). | rel-(R or S)-5-(5- (dimethylcarbamoyl) pyridin-2-yl)-1-isopropyl-N- (4- (methylcarbamoyl)benzyl)- 7-(trifluoromethyl)- 4,5,6,7-tetrahydro-1H- pyrazolo[4,3-c]pyridine- 3-carboxamide | 572.2 |
| 358 | Ex. 77^{e} | 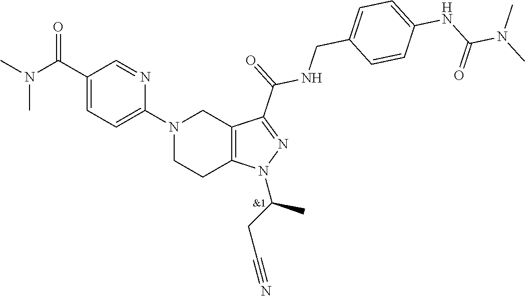 ¹H NMR (400 MHz, (CD₃)₂SO) δ 8.37 (t, 1H), 8.25-8.20 (m, 2H), 7.63 (dd, 1H), 7.41-7.35 (m, 2H), 7.20-7.13 (m, 2H), 6.89 (d, 1H), 4.80-4.68 (m, 3H), 4.35 (d, 2H), 3.95 (t, 2H), 3.06 (d, 2H), 2.96 (s, 6H), 2.91-2.82 (m, 8H), 1.46 (d, 3H). | rac-(R)-1-(1- cyanopropan-2-yl)-5-(5- (dimethylcarbamoyl) pyridin-2-yl)-N-(4-(3,3- dimethylureido)benzyl)- 4,5,6,7-tetrahydro-1H- pyrazolo[4,3-c]pyridine- 3-carboxamide | 558.3 |
| 359^{∧} | Ex. 353 | 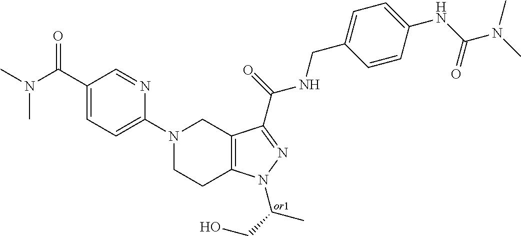 ¹H NMR (400 MHz, (CD₃)₂SO) δ 8.34 (t, 1H), 8.21-8.19 (m, 2H), 7.60 (dd, 1H), 7.37-7.32 (m, 2H), 7.17-7.11 (m, 2H), 6.85 (d, 1H), 4.84 (t, 1H), 4.78-4.64 (m, 2H), 4.36-4.22 (m, 3H), 3.97-3.83 (m, 2H), 3.61-3.54 (m, 2H), 2.93 (s, 6H), 2.86 (s, 6H), 2.81-2.76 (m, 2H), 1.33-1.30 (m, 3H). | rel-(R or S)-5-(5- (dimethylcarbamoyl) pyridin-2-yl)-N-(4-(3,3- dimethylureido)benzyl)- 1-(1-hydroxypropan-2- yl)-4,5,6,7-tetrahydro- 1H-pyrazolo[4,3- c]pyridine-3- carboxamide | 549.3 |
| 360^{∧} | Ex. 353 | 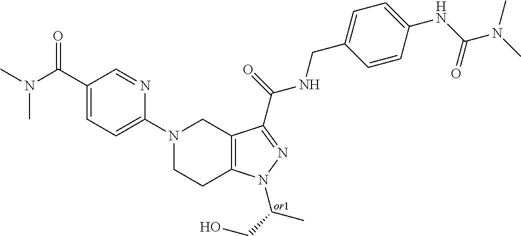 ¹H NMR (400 MHz, (CD₃)₂SO) δ 8.33 (t, 1H), 8.21-8.18 (m, 2H), 7.59 (dd, 1H), 7.38-7.32 (m, 2H), 7.16-7.11 (m, 2H), 6.84 (d, 1H), 4.83 (t, 1H), 4.78-4.64 (m, 2H), 4.37-4.22 (m, 3H), 3.97-3.82 (m, 2H), 3.61-3.53 (m, 2H), 2.93 (s, 6H), 2.86 (s, 6H), 2.81-2.75 (m, 2H), 1.37-1.32- 1.29 (m, 3H). | rel-(R or S)-5-(5- (dimethylcarbamoyl) pyridin-2-yl)-N-(4-(3,3- dimethylureido)benzyl)- 1-(1-hydroxypropan-2- yl)-4,5,6,7-tetrahydro- 1H-pyrazolo[4,3- c]pyridine-3- carboxamide | 549.3 |
| 361^{∧} | Ex. 322 | 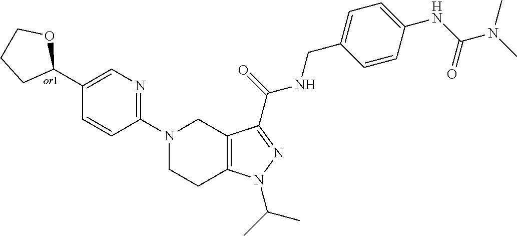 [SMILES: CC(C)n1nc(C(=O)NCc2ccc(NC(=O)N(C)C)cc2)c2c1CCN(c1ccc([C@H]3CCCO3)cn1)C2] ¹H NMR (400 MHz, CDCl₃) δ 8.12 (s, 1H), 7.53-7.43 (m, 1H), 7.38-7.23 (m, 4H), 7.17-7.10 (m, 1H), 6.84-6.75 (m, 1H), 6.31 (s, 1H), 4.81-4.64 (m, 3H), 4.60- 4.50 (m, 2H), 4.42-4.30 (m, 1H), 4.14- 3.99 (m, 3H), 3.93-3.83 (m, 1H), 3.03 (s, 6H), 2.83-2.73 (m, 2H), 2.30-2.17 (m, 1H), 2.08-1.95 (m, 2H), 1.85-1.73 (m, 1H), 1.49-1.38 (m, 6H). | rel-(R or S)-N-(4-(3,3- dimethylureido)benzyl)- 1-isopropyl-5-(5- (tetrahydrofuran-2- yl)pyridin-2-yl)-4,5,6,7- tetrahydro-1H- pyrazolo[4,3-c]pyridine- 3-carboxamide | 532.2 |
| 362^{∧} | Ex. 322 | 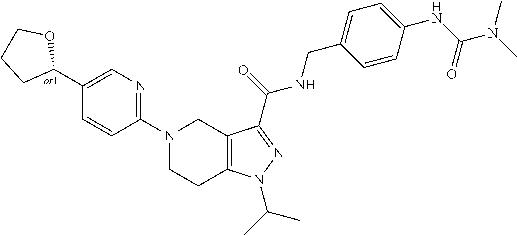 [SMILES: CC(C)n1nc(C(=O)NCc2ccc(NC(=O)N(C)C)cc2)c2c1CCN(c1ccc([C@@H]3CCCO3)cn1)C2] ¹H NMR (400 MHz, CDCl₃) δ 8.16-8.09 (m, 1H), 7.52-7.44 (m, 1H), 7.38-7.24 (m, 4H), 7.18-7.10 (m, 1H), 6.84-6.75 (m, 1H), 6.33-6.26 (m, 1H), 4.81-4.65 (m, 3H), 4.60-4.50 (m, 2H), 4.42-4.31 (m, 1H), 4.14-3.97 (m, 3H), 3.93-3.82 (m, 1H), 3.03 (s, 6H), 2.83-2.73 (m, 2H), 2.30-2.19 (m, 1H), 2.07-1.94 (m, 2H), 1.86-1.75 (m, 1H), 1.44 (s, 6H). | rel-(R or S)-N-(4-(3,3- dimethylureido)benzyl)- 1-isopropyl-5-(5- (tetrahydrofuran-2- yl)pyridin-2-yl)-4,5,6,7- tetrahydro-1H- pyrazolo[4,3-c]pyridine- 3-carboxamide | 532.3 |
| 363 | Ex. 193 | 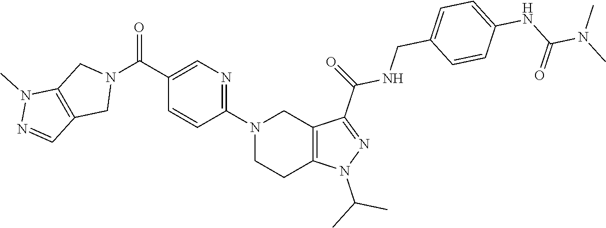 | N-(4-(3,3- dimethylureido)benzyl)- 1-isopropyl-5-(5-(1- methyl-1,4,5,6- tetrahydropyrrolo[3,4- c]pyrazole-5- carbonyl)pyridin-2-yl)- 4,5,6,7-tetrahydro-1H- pyrazolo[4,3-c]pyridine- 3-carboxamide | 611.8 |
| 364 | Ex. 193 | 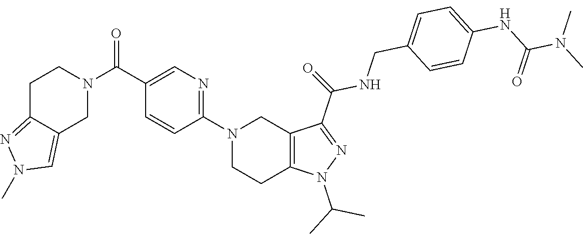 | N-(4-(3,3- dimethylureido)benzyl)- 1-isopropyl-5-(5-(2- methyl-4,5,6,7- tetrahydro-2H- pyrazolo[4,3-c]pyridine- 5-carbonyl)pyridin-2-yl)- 4,5,6,7-tetrahydro-1H- pyrazolo[4,3-c]pyridine- 3-carboxamide | 625.5 |
| 365 | Ex. 193 | 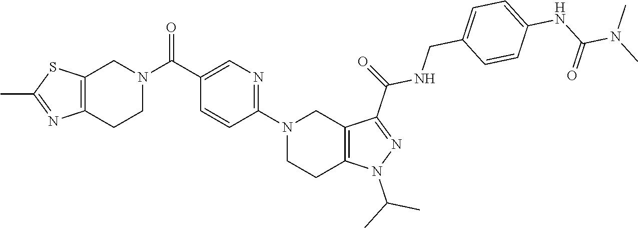 | N-(4-(3,3- dimethylureido)benzyl)- 1-isopropyl-5-(5-(2- methyl-4,5,6,7- tetrahydrothiazolo[5,4- c]pyridine-5- carbonyl)pyridin-2-yl)- 4,5,6,7-tetrahydro-1H- pyrazolo[4,3-c]pyridine- 3-carboxamide | 642.7 |
| 366 | Ex. 193 | 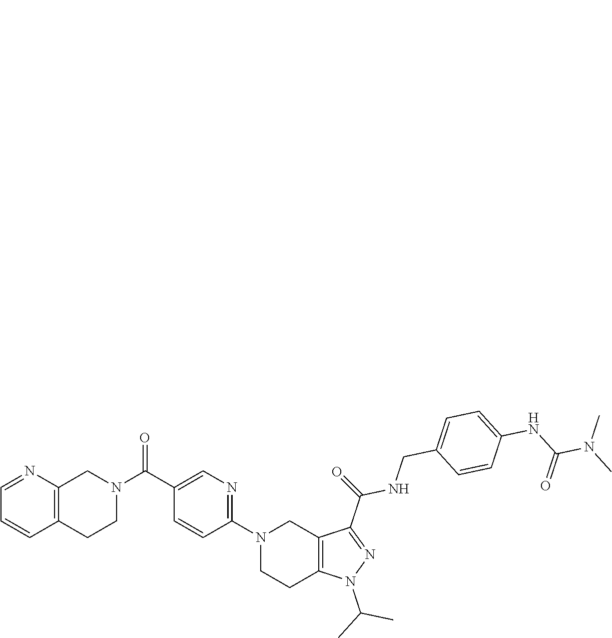 [SMILES: CC(C)n1nc(C(=O)NCc2ccc(NC(=O)N(C)C)cc2)c2c1CCN(c1ccc(C(=O)N3CCc4cccnc4C3)cn1)C2] | N-(4-(3,3- dimethylureido)benzyl)- 1-isopropyl-5-(5- (5,6,7,8- tetrahydropyrido[3,4- b]pyrazine-6- carbonyl)pyridin-2-yl)- 4,5,6,7-tetrahydro-1H- pyrazolo[4,3-c]pyridine- 3-carboxamide | 623.7 |
| 367 | Ex. 193 | 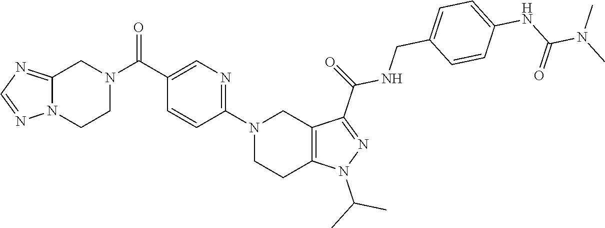 [SMILES: CC(C)n1nc(C(=O)NCc2ccc(NC(=O)N(C)C)cc2)c2c1CCN(c1ccc(C(=O)N3CCn4ncnc4C3)cn1)C2] | N-(4-(3,3- dimethylureido)benzyl)- 1-isopropyl-5-(5- (5,6,7,8-tetrahydro- [1,2,4]triazolo[1,5- a]pyrazine-7- carbonyl)pyridin-2-yl)- 4,5,6,7-tetrahydro-1H- pyrazolo[4,3-c]pyridine- 3-carboxamide | 612.6 |
| 368 | Ex. 193 | 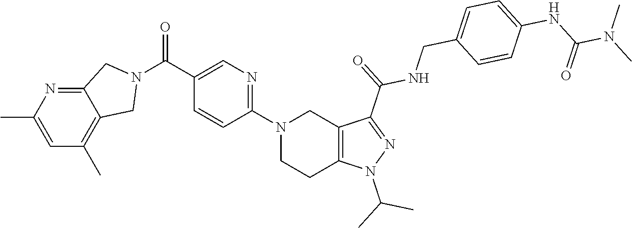 | 5-(5-(2,4-dimethyl-6,7- dihydro-5H-pyrrolo[3,4- b]pyridine-6- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 636.7 |
| 369 | Ex. 193 | 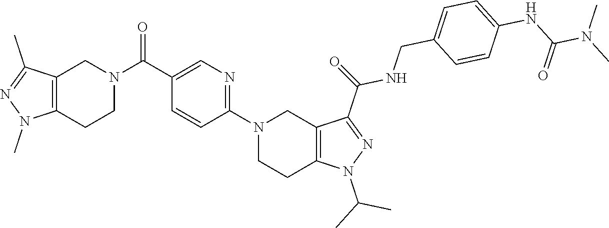 [SMILES: Cc1nn(C)c2c1CN(C(=O)c1ccc(N3CCc4c(c(C(=O)NCc5ccc(NC(=O)N(C)C)cc5)nn4C(C)C)C3)nc1)CC2] | 5-(5-(1,3-dimethyl- 4,5,6,7-tetrahydro-1H- pyrazolo[4,3-c]pyridine- 5-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 639.7 |
| 370 | Ex. 193 | 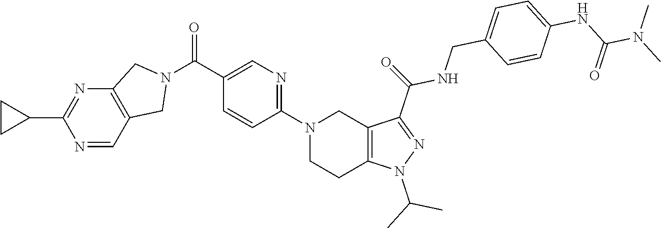 [SMILES: CC(C)n1nc(C(=O)NCc2ccc(NC(=O)N(C)C)cc2)c2c1CCN(c1ccc(C(=O)N3Cc4cnc(C5CC5)nc4C3)cn1)C2] | 5-(5-(2-cyclopropyl-6,7- dihydro-5H-pyrrolo[3,4- d]pyrimidine-6- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 649.7 |
| 371 | Ex. 193 | 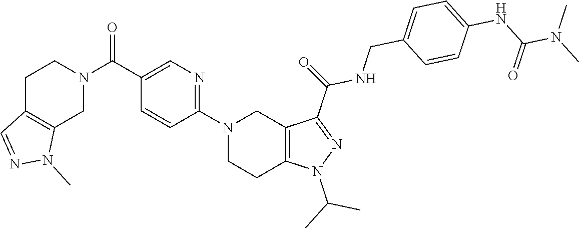 [SMILES: CC(C)n1nc(C(=O)NCc2ccc(NC(=O)N(C)C)cc2)c2c1CCN(c1ccc(C(=O)N3CCc4cnn(C)c4C3)cn1)C2] | N-(4-(3,3- dimethylureido)benzyl)- 1-isopropyl-5-(5-(1- methyl-4,5,6,7- tetrahydro-1H- pyrazolo[3,4-c]pyridine- 6-carbonyl)pyridin-2-yl)- 4,5,6,7-tetrahydro-1H- pyrazolo[4,3-c]pyridine- 3-carboxamide | 625.7 |
| 372 | Ex. 193 | 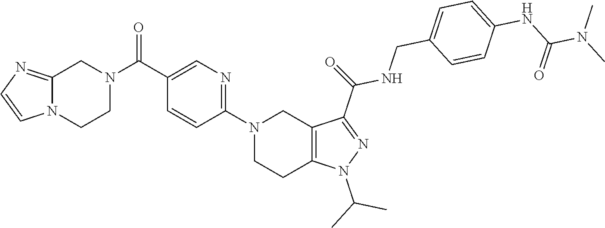 [SMILES: CC(C)n1nc(C(=O)NCc2ccc(NC(=O)N(C)C)cc2)c2c1CCN(c1ccc(C(=O)N3CCn4ccnc4C3)cn1)C2] | N-(4-(3,3- dimethylureido)benzyl)- 1-isopropyl-5-(5- (5,6,7,8- tetrahydroimidazo[1,2- a]pyrazine-7- carbonyl)pyridin-2-yl)- 4,5,6,7-tetrahydro-1H- pyrazolo[4,3-c]pyridine- 3-carboxamide | 611.7 |
| 373 | Ex. 193 | 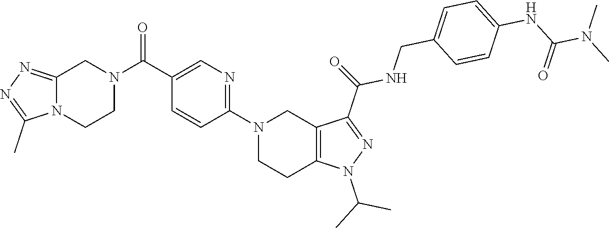 [SMILES: Cc1nnc2n1CCN(C(=O)c1ccc(N3CCc4c(c(C(=O)NCc5ccc(NC(=O)N(C)C)cc5)nn4C(C)C)C3)nc1)C2] | N-(4-(3,3- dimethylureido)benzyl)- 1-isopropyl-5-(5-(3- methyl-5,6,7,8- tetrahydro- [1,2,4]triazolo[4,3- a]pyrazine-7- carbonyl)pyridin-2-yl)- 4,5,6,7-tetrahydro-1H- pyrazolo[4,3-c]pyridine- 3-carboxamide | 626.7 |
| 374 | Ex. 193 | 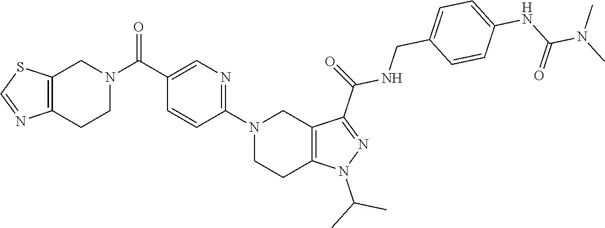 [SMILES: CC(C)n1nc(C(=O)NCc2ccc(NC(=O)N(C)C)cc2)c2c1CCN(c1ccc(C(=O)N3CCc4ncsc4C3)cn1)C2] | N-(4-(3,3- dimethylureido)benzyl)- 1-isopropyl-5-(5- (4,5,6,7- tetrahydrothiazolo[5,4- c]pyridine-5- carbonyl)pyridin-2-yl)- 4,5,6,7-tetrahydro-1H- pyrazolo[4,3-c]pyridine- 3-carboxamide | 628.6 |
| 375 | Ex. 193 | 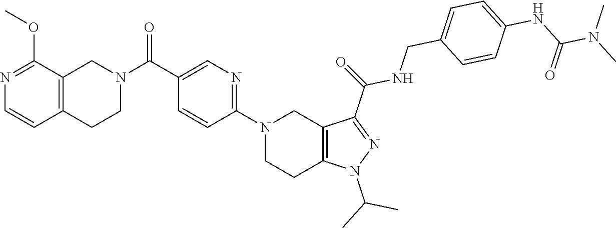 [SMILES: COc1nccc2c1CN(C(=O)c1ccc(N3CCc4c(c(C(=O)NCc5ccc(NC(=O)N(C)C)cc5)nn4C(C)C)C3)nc1)CC2] | N-(4-(3,3- dimethylureido)benzyl)- 1-isopropyl-5-(5-(8- methoxy-1,2,3,4- tetrahydro-2,7- naphthyridine-2- carbonyl)pyridin-2-yl)- 4,5,6,7-tetrahydro-1H- pyrazolo[4,3-c]pyridine- 3-carboxamide | 652.8 |
| 376 | Ex. 193 | 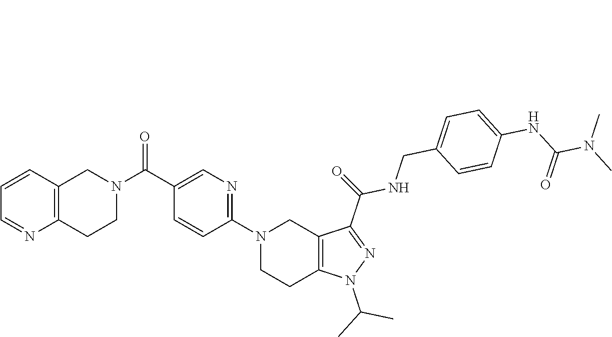 [SMILES: CC(C)n1nc(C(=O)NCc2ccc(NC(=O)N(C)C)cc2)c2c1CCN(c1ccc(C(=O)N3CCc4ncccc4C3)cn1)C2] | N-(4-(3,3- dimethylureido)benzyl)- 1-isopropyl-5-(5- (5,6,7,8-tetrahydro-1,6- naphthyridine-6- carbonyl)pyridin-2-yl)- 4,5,6,7-tetrahydro-1H- pyrazolo[4,3-c]pyridine- 3-carboxamide | 622.7 |
| 377 | Ex. 193 | 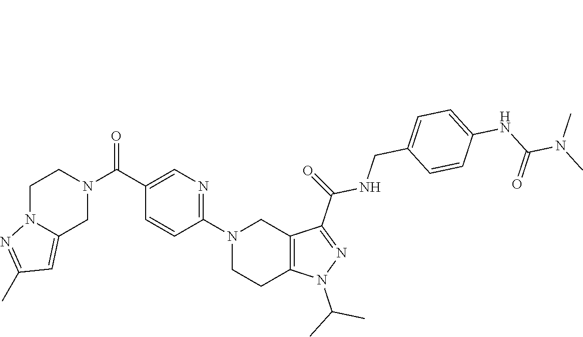 | N-(4-(3,3- dimethylureido)benzyl)- 1-isopropyl-5-(5-(2- methyl-4,5,6,7- tetrahydropyrazolo[1,5- a]pyrazine-5- carbonyl)pyridin-2-yl)- 4,5,6,7-tetrahydro-1H- pyrazolo[4,3-c]pyridine- 3-carboxamide | 625.7 |
| 378 | Ex. 193 | 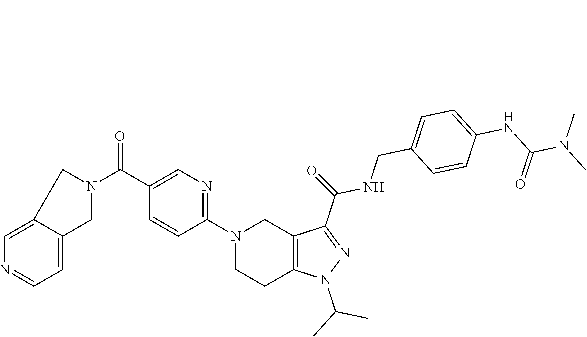 [SMILES: CC(C)n1nc(C(=O)NCc2ccc(NC(=O)N(C)C)cc2)c2c1CCN(c1ccc(C(=O)N3Cc4ccncc4C3)cn1)C2] | 5-(5-(2,3-dihydro-1H- pyrrolo[3,4-c]pyridine-2- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 608.6 |
| 379 | Ex. 193 | 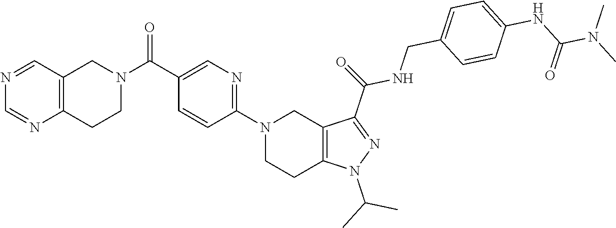 [SMILES: CC(C)n1nc(C(=O)NCc2ccc(NC(=O)N(C)C)cc2)c2c1CCN(c1ccc(C(=O)N3CCc4ncncc4C3)cn1)C2] | N-(4-(3,3- dimethylureido)benzyl)- 1-isopropyl-5-(5- (5,6,7,8- tetrahydropyrido[4,3- d]pyrimidine-6- carbonyl)pyridin-2-yl)- 4,5,6,7-tetrahydro-1H- pyrazolo[4,3-c]pyridine- 3-carboxamide | 623.7 |
| 380 | Ex. 193 | 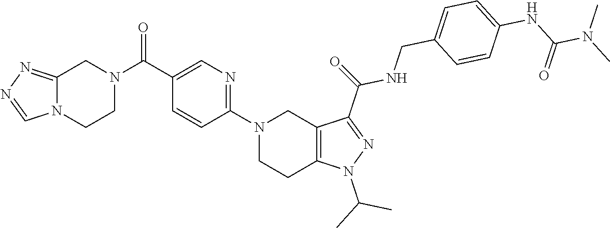 [SMILES: CC(C)n1nc(C(=O)NCc2ccc(NC(=O)N(C)C)cc2)c2c1CCN(c1ccc(C(=O)N3CCn4cnnc4C3)cn1)C2] | N-(4-(3,3- dimethylureido)benzyl)- 1-isopropyl-5-(5- (5,6,7,8-tetrahydro- [1,2,4]triazolo[4,3- a]pyrazine-7- carbonyl)pyridin-2-yl)- 4,5,6,7-tetrahydro-1H- pyrazolo[4,3-c]pyridine- 3-carboxamide | 612.7 |
| 381 | Ex. 193 | 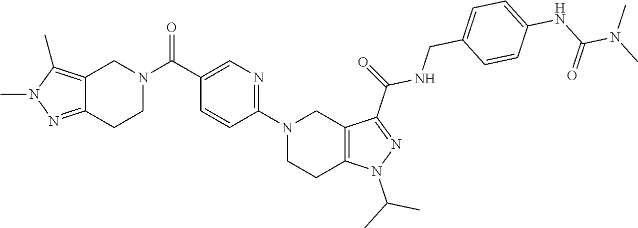 | 5-(5-(2,3-dimethyl- 4,5,6,7-tetrahydro-2H- pyrazolo[4,3-c]pyridine- 5-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 639.6 |
| 382 | Ex. 193 | 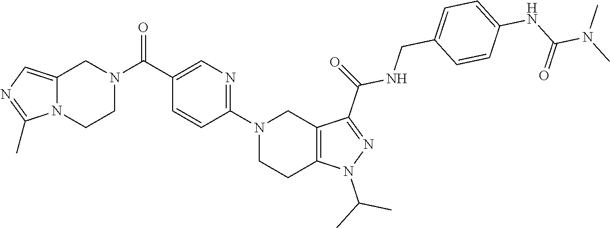 [SMILES: Cc1ncc2n1CCN(C(=O)c1ccc(N3CCc4c(c(C(=O)NCc5ccc(NC(=O)N(C)C)cc5)nn4C(C)C)C3)nc1)C2] | N-(4-(3,3- dimethylureido)benzyl)- 1-isopropyl-5-(5-(3- methyl-5,6,7,8- tetrahydroimidazo[1,5- a]pyrazine-7- carbonyl)pyridin-2-yl)- 4,5,6,7-tetrahydro-1H- pyrazolo[4,3-c]pyridine- 3-carboxamide | 625.7 |
| 383 | Ex. 193 | 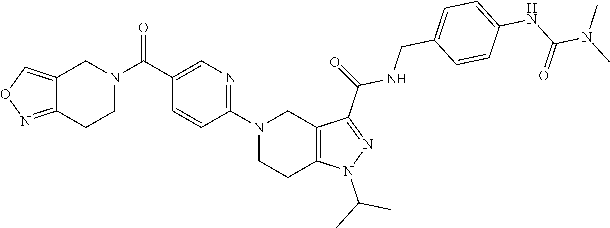 [SMILES: CC(C)n1nc(C(=O)NCc2ccc(NC(=O)N(C)C)cc2)c2c1CCN(c1ccc(C(=O)N3CCc4nocc4C3)cn1)C2] | N-(4-(3,3- dimethylureido)benzyl)- 1-isopropyl-5-(5- (4,5,6,7- tetrahydroisoxazolo[4,3- c]pyridine-5- carbonyl)pyridin-2-yl)- 4,5,6,7-tetrahydro-1H- pyrazolo[4,3-c]pyridine- 3-carboxamide | 612.5 |
| 384 | Ex. 193 | 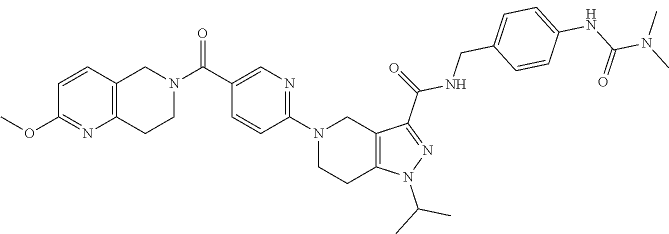 | N-(4-(3,3- dimethylureido)benzyl)- 1-isopropyl-5-(5-(2- methoxy-5,6,7,8- tetrahydro-1,6- naphthyridine-6- carbonyl)pyridin-2-yl)- 4,5,6,7-tetrahydro-1H- pyrazolo[4,3-c]pyridine- 3-carboxamide | 652.7 |
| 385 | Ex. 193 | 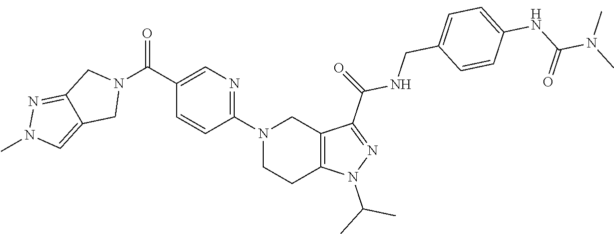 | N-(4-(3,3- dimethylureido)benzyl)- 1-isopropyl-5-(5-(2- methyl-2,4,5,6- tetrahydropyrrolo[3,4- c]pyrazole-5- carbonyl)pyridin-2-yl)- 4,5,6,7-tetrahydro-1H- pyrazolo[4,3-c]pyridine- 3-carboxamide | 611.7 |
| 386 | Ex. 193 | 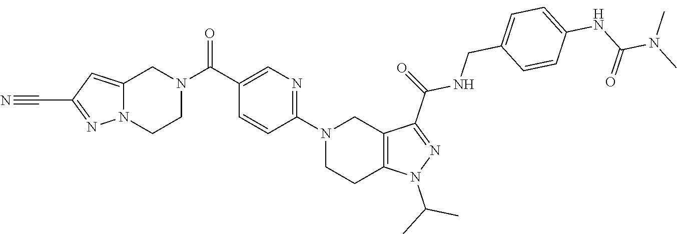 [SMILES: CC(C)n1nc(C(=O)NCc2ccc(NC(=O)N(C)C)cc2)c2c1CCN(c1ccc(C(=O)N3CCn4nc(C#N)cc4C3)cn1)C2] | 5-(5-(2-cyano-4,5,6,7- tetrahydropyrazolo[1,5- a]pyrazine-5- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 636.7 |
| 387 | Ex. 193 | 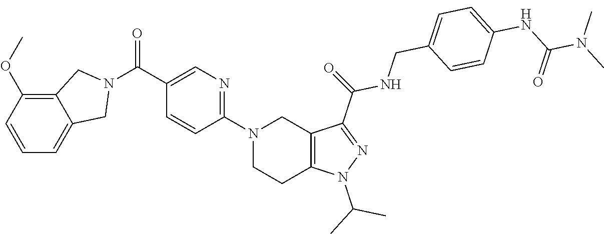 [SMILES: COc1cccc2c1CN(C(=O)c1ccc(N3CCc4c(c(C(=O)NCc5ccc(NC(=O)N(C)C)cc5)nn4C(C)C)C3)nc1)C2] | N-(4-(3,3- dimethylureido)benzyl)- 1-isopropyl-5-(5-(4- methoxyisoindoline-2- carbonyl)pyridin-2-yl)- 4,5,6,7-tetrahydro-1H- pyrazolo[4,3-c]pyridine- 3-carboxamide | 637.7 |
| 388 | Ex. 77 | 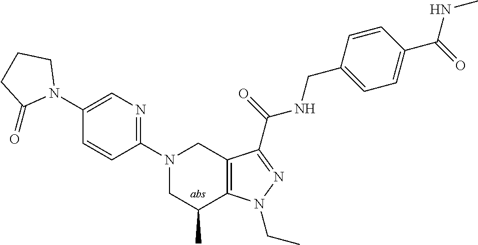 [SMILES: CCn1nc(C(=O)NCc2ccc(C(=O)NC)cc2)c2c1[C@@H](C)CN(c1ccc(N3CCCC3=O)cn1)C2] ¹H NMR (400 MHz, (CD₃)₂SO) δ 8.64 (t, 1H), 8.40-8.34 (m, 1H), 8.28 (d, 1H), 7.87 (dd, 1H), 7.79-7.72 (m, 2H), 7.40-7.33 (m, 2H), 6.89 (d, 1H), 4.89 (d, 1H), 4.49- 4.37 (m, 2H), 4.28-4.06 (m, 4H), 3.79- 3.73 (m, 2H), 3.23-3.15 (m, 1H), 2.77 (d, 3H), 2.47-2.40 (m, 3H), 2.12-2.00 (m, 2H), 1.39 (t, 3H), 1.21 (d, 3H). | (S)-1-ethyl-7-methyl-N- (4- (methylcarbamoyl)benzyl)- 5-(5-(2-oxopyrrolidin- 1-yl)pyridin-2-yl)-4,5,6,7- tetrahydro-1H- pyrazolo[4,3-c]pyridine- 3-carboxamide | 516.3 |
| 389 | Ex. 77 | 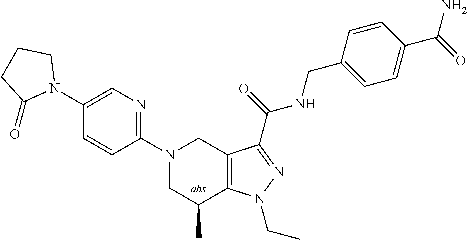 [SMILES: CCn1nc(C(=O)NCc2ccc(C(N)=O)cc2)c2c1[C@@H](C)CN(c1ccc(N3CCCC3=O)cn1)C2] ¹H NMR (400 MHz, (CD₃)₂SO) δ 8.64 (t, 1H), 8.28 (d, 1H), 7.93-7.85 (m, 2H), 7.84- 7.80 (m, 2H), 7.39-7.28 (m, 3H), 6.89 (d, 1H), 4.90 (d, 1H), 4.49-4.41 (m, 2H), 4.30- 4.07 (m, 4H), 3.80-3.74 (m, 2H), 3.24- 3.16 (m, 1H), 2.47-2.39 (m, 3H), 2.12- 1.99 (m, 2H), 1.40 (t, 3H), 1.22 (d, 3H). | (S)-N-(4- carbamoylbenzyl)-1- ethyl-7-methyl-5-(5-(2- oxopyrrolidin-1- yl)pyridin-2-yl)-4,5,6,7- tetrahydro-1H- pyrazolo[4,3-c]pyridine- 3-carboxamide | 502.2 |
| 390 | Ex. 39^{f} | 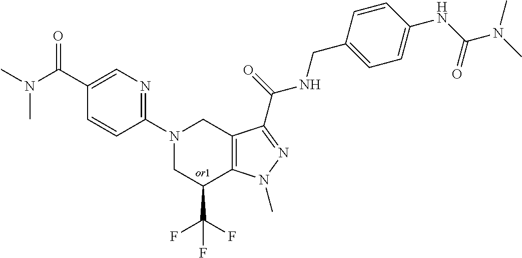 ¹H NMR (600 MHz, (CD₃)₂SO) δ 8.75 (t, 1H), 8.28-8.26 (m, 2H), 7.72-7.69 (m, 1H), 7.48-7.44 (m, 2H), 7.26-7.22 (m, 2H), 6.86-6.82 (m, 1H), 4.89 (d, 1H), 4.63 (dd, 1H), 4.51 (d, 1H), 4.43 (d, 2H), 4.05- 3.98 (m, 1H), 3.96 (s, 3H), 3.82 (dd, 1H), 2.98 (s, 6H), 2.93-2.90 (m, 6H). | rel-(R or S)-5-(5- (dimethylcarbamoyl)pyridin- 2-yl)-N-(4-(3,3- dimethylureido)benzyl)- 1-methyl-7- (trifluoromethyl)-4,5,6,7- tetrahydro-1H- pyrazolo[4,3-c]pyridine- 3-carboxamide | 573.2 |
| 391^{∧} | Ex. 39^{f} | 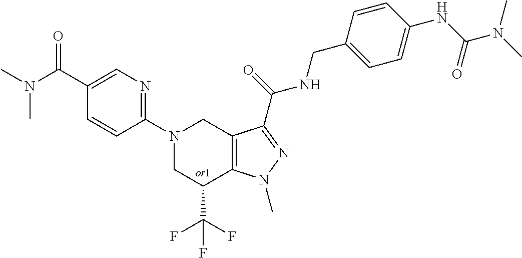 ¹H NMR (600 MHz, (CD₃)₂SO) δ 8.75 (t, 1H), 8.27 (s, 2H), 7.71 (dd, 1H), 7.48-7.43 (m, 2H), 7.26-7.22 (m, 2H), 6.84 (d, 1H), 4.89 (d, 1H), 4.63 (dd, 1H), 4.51 (d, 1H), 4.43 (d, 2H), 4.05-3.98 (m, 1H), 3.96 (s, 3H), 3.82 (dd, 1H), 2.98 (s, 6H), 2.92 (s, 6H). | rel-(R or S)-5-(5- (dimethylcarbamoyl) pyridin-2-yl)-N-(4-(3,3- dimethylureido)benzyl)- 1-methyl-7- (trifluoromethyl)-4,5,6,7- tetrahydro-1H- pyrazolo[4,3-c]pyridine- 3-carboxamide | 573.2 |
| 392^{∧} | Ex. 39^{f} | 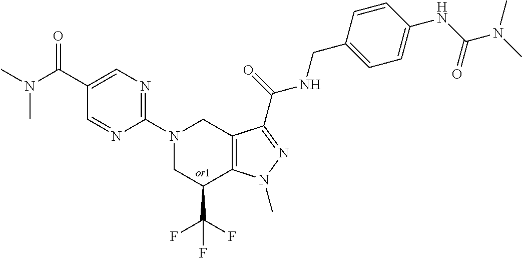 ¹H NMR (600 MHz, (CD₃)₂SO) δ 8.83 (t, 1H), 8.56 (s, 2H), 8.26 (s, 1H), 7.47-7.42 (m, 2H), 7.25-7.20 (m, 2H), 5.27 (d, 1H), 4.90 (dd, 1H), 4.67 (d, 1H), 4.42 (d, 2H), 4.06-3.98 (m, 1H), 3.96 (s, 3H), 3.88 (dd, 1H), 3.06-2.94 (m, 6H), 2.92 (s, 6H). | rel-(R or S)-5-(5- (dimethylcarbamoyl) pyrimidin-2-yl)-N-(4-(3,3- dimethylureido)benzyl)- 1-methyl-7- (trifluoromethyl)-4,5,6,7- tetrahydro-1H- pyrazolo[4,3-c]pyridine- 3-carboxamide | 574.2 |
| 393^{∧} | Ex. 322 | 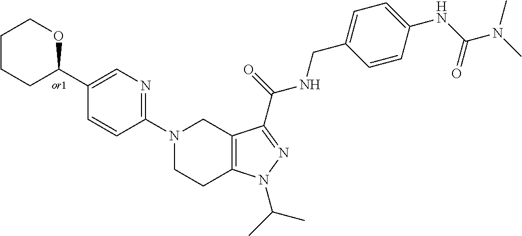 [SMILES: CC(C)n1nc(C(=O)NCc2ccc(NC(=O)N(C)C)cc2)c2c1CCN(c1ccc([C@H]3CCCCO3)cn1)C2] ¹H NMR (400 MHz, CDCl₃) δ 8.14-8.11 (m, 1H), 7.55-7.50 (m, 1H), 7.38-7.32 (m, 2H), 7.31-7.28 (m, 2H), 7.17-7.10 (m, 1H), 6.82-6.77 (m, 1H), 6.31-6.26 (m, 1H), 4.68 (s, 2H), 4.56 (d, 2H), 4.42- 4.30 (m, 1H), 4.26-4.20 (m, 1H), 4.12- 4.04 (m, 3H), 3.63-3.53 (m, 1H), 3.03 (s, 6H), 2.81-2.75 (m, 2H), 1.96-1.90 (m, 1H), 1.79-1.72 (m, 1H), 1.68-1.62 (m, 3H), 1.55-1.52 (m, 1H), 1.43 (d, 6H). | rel-(R or S)-N-(4-(3,3- dimethylureido)benzyl)- 1-isopropyl-5-(5- (tetrahydro-2H-pyran-2- yl)pyridin-2-yl)-4,5,6,7- tetrahydro-1H- pyrazolo[4,3-c]pyridine- 3-carboxamide | 546.3 |
| 394^{∧} | Ex. 322 | 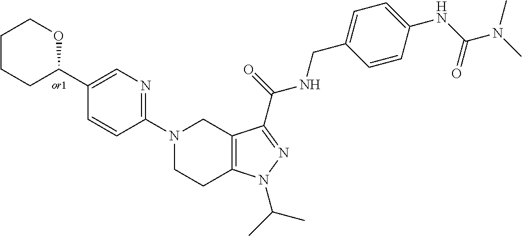 [SMILES: CC(C)n1nc(C(=O)NCc2ccc(NC(=O)N(C)C)cc2)c2c1CCN(c1ccc([C@@H]3CCCCO3)cn1)C2] ¹H NMR (400 MHz, CDCl₃) δ 8.13-8.11 (m, 1H), 7.54-7.50 (m, 1H), 7.38-7.33 (m, 2H), 7.31-7.28 (m, 2H), 7.16-7.11 (m, 1H), 6.79 (d, 1H), 6.29 (s, 1H), 4.68 (s, 2H), 4.56 (d, 2H), 4.40-4.31 (m, 1H), 4.25- 4.20 (m, 1H), 4.12-4.04 (m, 3H), 3.63- 3.54 (m, 1H), 3.03 (s, 6H), 2.78 (t, 2H), 1.96- 1.89 (m, 1H), 1.79-1.73 (m, 1H), 1.68- 1.61 (m, 3H), 1.55-1.52 (m, 1H), 1.43 (d, 6H). | rel-(R or S)-N-(4-(3,3- dimethylureido)benzyl)- 1-isopropyl-5-(5- (tetrahydro-2H-pyran-2- yl)pyridin-2-yl)-4,5,6,7- tetrahydro-1H- pyrazolo[4,3-c]pyridine- 3-carboxamide | 546.3 |
| 395 | Ex. 395 | 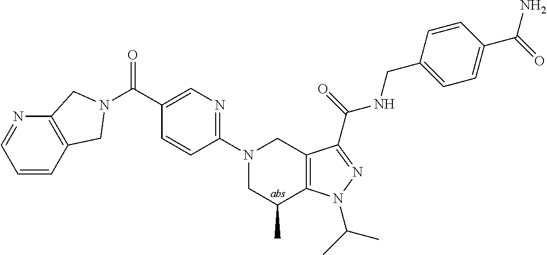 [SMILES: CC(C)n1nc(C(=O)NCc2ccc(C(N)=O)cc2)c2c1[C@@H](C)CN(c1ccc(C(=O)N3Cc4cccnc4C3)cn1)C2] ¹H NMR (400 MHz, (CD₃)₂SO) δ 8.55 (t, 1H), 8.50-8.41 (m, 2H), 7.92-7.75 (m, 5H), 7.36 (d, 2H), 7.32-7.26 (m, 2H), 6.95- 6.90 (m, 1H), 5.12 (d, 1H), 4.99-4.78 (m, 4H), 4.60-4.40 (m, 4H), 4.25 (d, 1H), 3.28- 3.20 (m, 2H), 1.47-1.38 (m, 6H), 1.20 (d, 3H). | (S)-N-(4- carbamoylbenzyl)-5-(5- (6,7-dihydro-5H- pyrrolo[3,4-b]pyridine-6- carbonyl)pyridin-2-yl)-1- isopropyl-7-methyl- 4,5,6,7-tetrahydro-1H- pyrazolo[4,3-c]pyridine- 3-carboxamide | 579.4 |
| 396 | Ex. 395 | 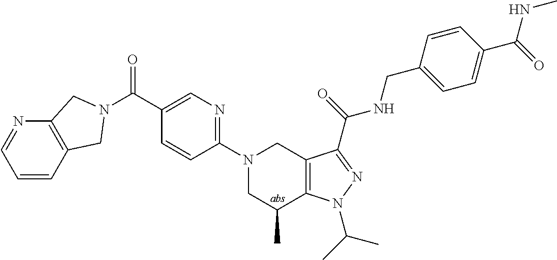 [SMILES: CNC(=O)c1ccc(CNC(=O)c2nn(C(C)C)c3c2CN(c2ccc(C(=O)N4Cc5cccnc5C4)cn2)C[C@@H]3C)cc1] ¹H NMR (400 MHz, (CD₃)₂SO) δ 8.56- 8.51 (m, 1H), 8.49-8.42 (m, 2H), 8.37- 8.31 (m, 1H), 7.90-7.79 (m, 1H), 7.78- 7.66 (m, 3H), 7.38-7.33 (m, 2H), 7.32- 7.26 (m, 1H), 6.93 (d, 1H), 5.11 (d, 1H), 4.98-4.76 (m, 4H), 4.58-4.38 (m, 4H), 4.25 (d, 1H), 3.28-3.18 (m, 2H), 2.74 (d, 3H), 1.45-1.37 (m, 6H), 1.19 (d, 3H). | (S)-5-(5-(6,7-dihydro- 5H-pyrrolo[3,4- b]pyridine-6- carbonyl)pyridin-2-yl)-1- isopropyl-7-methyl-N-(4- (methylcarbamoyl)benzyl)- 4,5,6,7-tetrahydro-1H- pyrazolo[4,3-c]pyridine- 3-carboxamide | 593.4 |
| 397 | Ex. 127 | 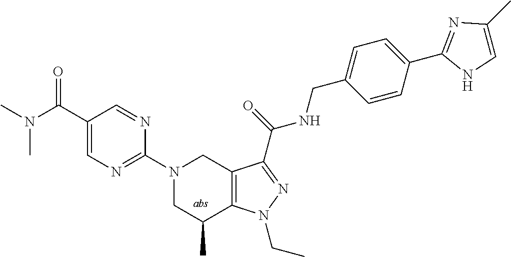 ¹H NMR (400 MHz, (CD₃)₂SO) δ 12.21- 12.09 (m, 1H), 8.60 (t, 1H), 8.51 (s, 2H), 7.84-7.77 (m, 2H), 7.33 (d, 2H), 6.92- 6.63 (m, 1H), 5.42 (d, 1H), 4.67 (dd, 1H), 4.49-4.35 (m, 3H), 4.17-4.04 (m, 2H), 3.51-3.45 (m, 1H), 3.28-3.20 (m, 1H), 2.99 (s, 6H), 2.18 (d, 3H), 1.39 (t, 3H), 1.16 (d, 3H). | (S)-5-(5- (dimethylcarbamoyl) pyrimidin-2-yl)-1-ethyl-7- methyl-N-(4-(4-methyl- 1H-imidazol-2- yl)benzyl)-4,5,6,7- tetrahydro-1H- pyrazolo[4,3-c]pyridine- 3-carboxamide | 528.3 |
| 398 | Ex. 537 | 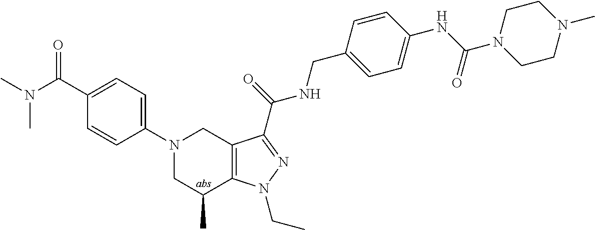 ¹H NMR (400 MHz, (CD₃)₂SO) δ 8.49- 8.41 (m, 2H), 7.43-7.29 (m, 4H), 7.18- 7.13 (m, 2H), 6.99-6.93 (m, 2H), 4.52 (d, 1H), 4.37-4.28 (m, 2H), 4.18-4.04 (m, 3H), 3.80-3.72 (m, 1H), 3.43-3.37 (m, 4H), 3.31-3.29 (m, 1H), 3.22-3.14 (m, 1H), 2.97-2.89 (m, 6H), 2.28 (t, 4H), 2.18 (s, 3H), 1.38 (t, 3H), 1.28 (d, 3H). | (S)-5-(4- (dimethylcarbamoyl) phenyl)-1-ethyl-7-methyl-N- (4-(4-methylpiperazine- 1-carboxamido)benzyl)- 4,5,6,7-tetrahydro-1H- pyrazolo[4,3-c]pyridine- 3-carboxamide | 587.3 |
| 399 | Ex. 537 | 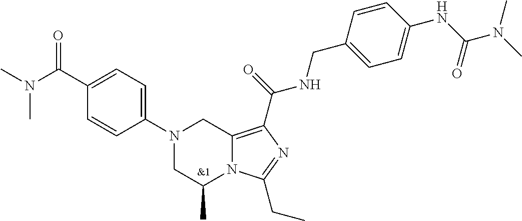 ¹H NMR (400 MHz, (CD₃)₂SO) δ 8.21 (s, 1H), 8.12 (t, 1H), 7.39-7.33 (m, 4H), 7.16- 7.12 (m, 2H), 7.00-6.96 (m, 2H), 4.90 (d, 1H), 4.58-4.48 (m, 1H), 4.39-4.27 (m, 3H), 3.99 (d, 1H), 3.43-3.37 (m, 1H), 2.95 (s, 6H), 2.89 (s, 6H), 2.77-2.60 (m, 2H), 1.38 (d, 3H), 1.26 (t, 3H). | rac-(R)-7-(4- (dimethylcarbamoyl) phenyl)-N-(4-(3,3- dimethylureido)benzyl)- 3-ethyl-5-methyl-5,6,7,8- tetrahydroimidazo[1,5- a]pyrazine-1- carboxamide | 532.7 |
| 400 | Ex. 23^{j} | 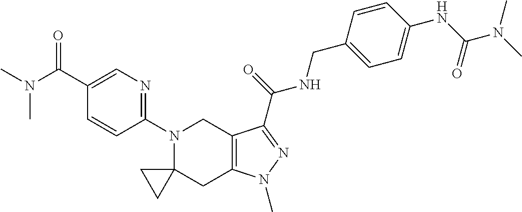 ¹H NMR (400 MHz, CD₃OD) δ 8.23-8.21 (m, 1H), 7.64 (dd, 1H), 7.34-7.29 (m, 2H), 7.27-7.23 (m, 2H), 7.18-7.14 (m, 1H), 5.99-5.90 (m, 1H), 4.52-4.39 (m, 2H), 4.26-4.16 (m, 1H), 3.70 (s, 3H), 3.53- 3.34 (m, 1H), 3.05 (s, 6H), 2.99 (s, 6H), 2.25-2.15 (m, 1H), 1.36-1.25 (m, 2H), 0.93-0.80 (m, 2H). | 5′-(5- (dimethylcarbamoyl) pyridin-2-yl)-N-(4-(3,3- dimethylureido)benzyl)- 1′-methyl-1′,4′,5′,7′- tetrahydrospiro [cyclopropane- 1,6′-pyrazolo[4,3- c]pyridine]-3′- carboxamide | 531.3 |
| 401 | Ex. 193 | 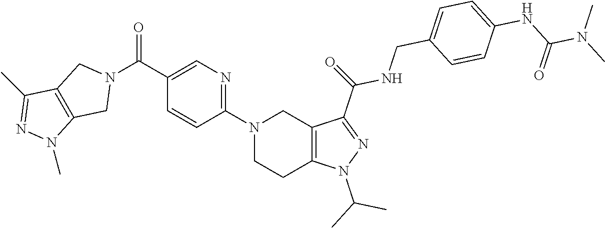 | 5-(5-(1,3-dimethyl- 1,4,5,6- tetrahydropyrrolo[3,4- c]pyrazole-5- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 625.6 |
| 402 | Ex. 193 | 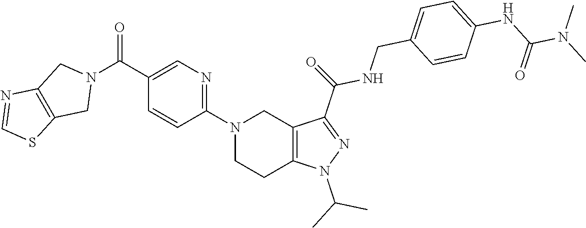 [SMILES: CC(C)n1nc(C(=O)NCc2ccc(NC(=O)N(C)C)cc2)c2c1CCN(c1ccc(C(=O)N3Cc4ncsc4C3)cn1)C2] | 5-(5-(5,6-dihydro-4H- pyrrolo[3,4-d]thiazole-5- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 614.8 |
| 403 | Ex. 193 | 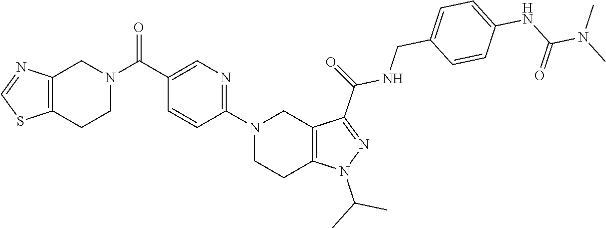 [SMILES: CC(C)n1nc(C(=O)NCc2ccc(NC(=O)N(C)C)cc2)c2c1CCN(c1ccc(C(=O)N3CCc4scnc4C3)cn1)C2] | N-(4-(3,3- dimethylureido)benzyl)- 1-isopropyl-5-(5- (4,5,6,7- tetrahydrothiazolo [4,5- c]pyridine-5- carbonyl)pyridin-2-yl)- 4,5,6,7-tetrahydro-1H- pyrazolo[4,3-c]pyridine- 3-carboxamide | 628.8 |
| 404 | Ex. 193 | 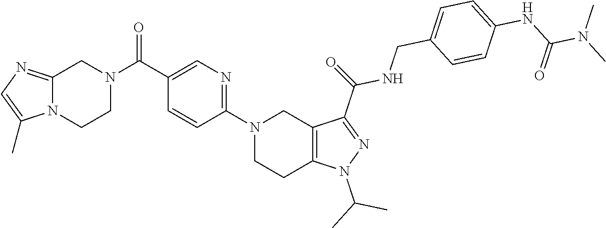 [SMILES: Cc1cnc2n1CCN(C(=O)c1ccc(N3CCc4c(c(C(=O)NCc5ccc(NC(=O)N(C)C)cc5)nn4C(C)C)C3)nc1)C2] | N-(4-(3,3- dimethylureido)benzyl)- 1-isopropyl-5-(5-(3- methyl-5,6,7,8- tetrahydroimidazo[1,2- a]pyrazine-7- carbonyl)pyridin-2-yl)- 4,5,6,7-tetrahydro-1H- pyrazolo[4,3-c]pyridine- 3-carboxamide | 625.6 |
| 405 | Ex. 193 | 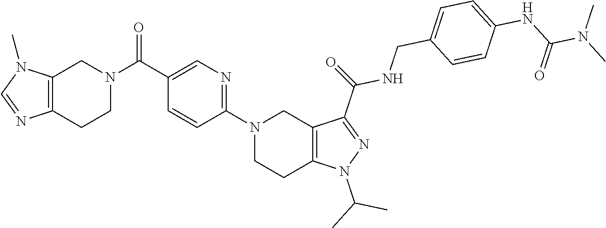 [SMILES: CC(C)n1nc(C(=O)NCc2ccc(NC(=O)N(C)C)cc2)c2c1CCN(c1ccc(C(=O)N3CCc4ncn(C)c4C3)cn1)C2] | N-(4-(3,3- dimethylureido)benzyl)- 1-isopropyl-5-(5-(3- methyl-4,5,6,7- tetrahydro-3H- imidazo[4,5-c]pyridine-5- carbonyl)pyridin-2-yl)- 4,5,6,7-tetrahydro-1H- pyrazolo[4,3-c]pyridine- 3-carboxamide | 625.7 |
| 406 | Ex. 193 | 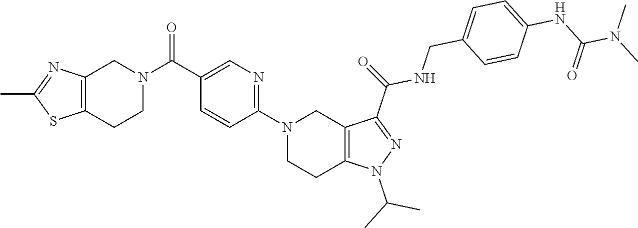 | N-(4-(3,3- dimethylureido)benzyl)- 1-isopropyl-5-(5-(2- methyl-4,5,6,7- tetrahydrothiazolo[4,5- c]pyridine-5- carbonyl)pyridin-2-yl)- 4,5,6,7-tetrahydro-1H- pyrazolo[4,3-c]pyridine- 3-carboxamide | 642.5 |
| 407 | Ex. 193 | 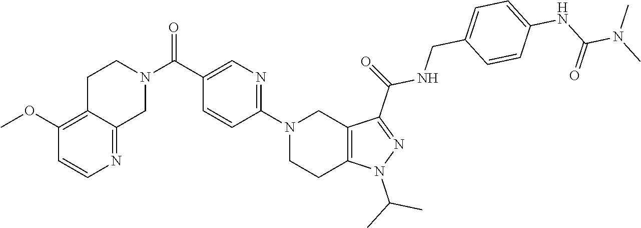 | N-(4-(3,3- dimethylureido)benzyl)- 1-isopropyl-5-(5-(4- methoxy-5,6,7,8- tetrahydro-1,7- naphthyridine-7- carbonyl)pyridin-2-yl)- 4,5,6,7-tetrahydro-1H- pyrazolo[4,3-c]pyridine- 3-carboxamide | 652.6 |
| 408 | Ex. 193 | 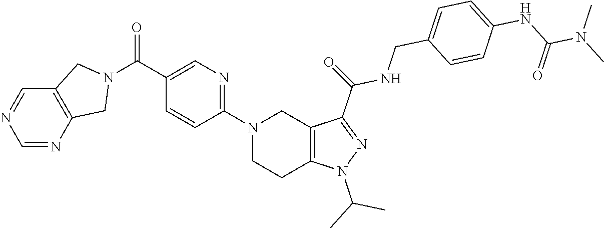 [SMILES: CC(C)n1nc(C(=O)NCc2ccc(NC(=O)N(C)C)cc2)c2c1CCN(c1ccc(C(=O)N3Cc4cncnc4C3)cn1)C2] | 5-(5-(6,7-dihydro-5H- pyrrolo[3,4-d]pyrimidine- 6-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 609.5 |
| 409 | Ex. 193 | 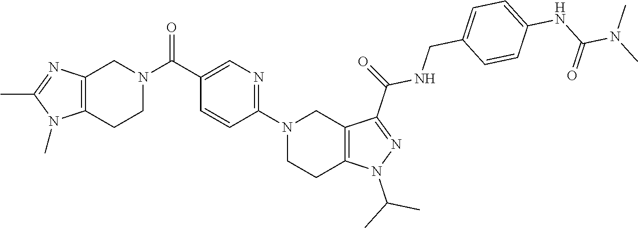 | 5-(5-(1,2-dimethyl- 4,5,6,7-tetrahydro-1H- imidazo[4,5-c]pyridine-5- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 639.6 |
| 410 | Ex. 193 | 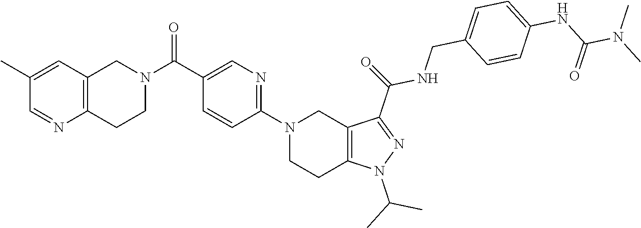 | N-(4-(3,3- dimethylureido)benzyl)- 1-isopropyl-5-(5-(3- methyl-5,6,7,8- tetrahydro-1,6- naphthyridine-6- carbonyl)pyridin-2-yl)- 4,5,6,7-tetrahydro-1H- pyrazolo[4,3-c]pyridine- 3-carboxamide | 636.6 |
| 411 | Ex. 193 | 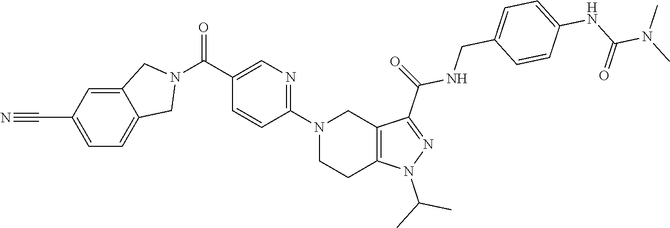 [SMILES: CC(C)n1nc(C(=O)NCc2ccc(NC(=O)N(C)C)cc2)c2c1CCN(c1ccc(C(=O)N3Cc4ccc(C#N)cc4C3)cn1)C2] | 5-(5-(5- cyanoisoindoline-2- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 632.8 |
| 412 | Ex. 193 | 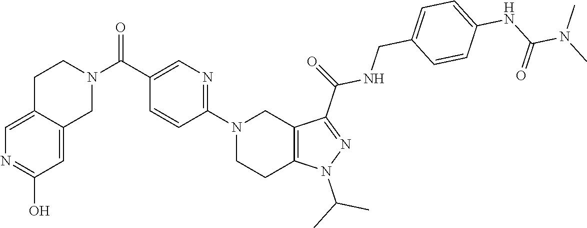 [SMILES: CC(C)n1nc(C(=O)NCc2ccc(NC(=O)N(C)C)cc2)c2c1CCN(c1ccc(C(=O)N3CCc4cnc(O)cc4C3)cn1)C2] | N-(4-(3,3- dimethylureido)benzyl)- 5-(5-(7-hydroxy-1,2,3,4- tetrahydro-2,6- naphthyridine-2- carbonyl)pyridin-2-yl)-1- isopropyl-4,5,6,7- tetrahydro-1H- pyrazolo[4,3-c]pyridine- 3-carboxamide | 638.6 |
| 413 | Ex. 193 | 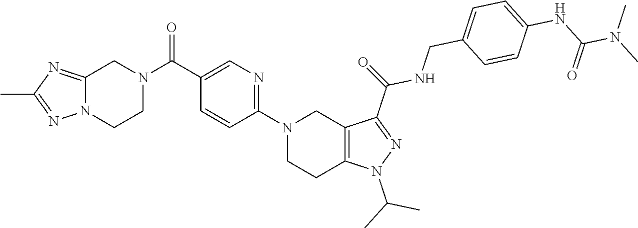 | N-(4-(3,3- dimethylureido)benzyl)- 1-isopropyl-5-(5-(2- methyl-5,6,7,8- tetrahydro- [1,2,4]triazolo[1,5- a]pyrazine-7- carbonyl)pyridin-2-yl)- 4,5,6,7-tetrahydro-1H- pyrazolo[4,3-c]pyridine- 3-carboxamide | 626.6 |
| 414 | Ex. 193 | 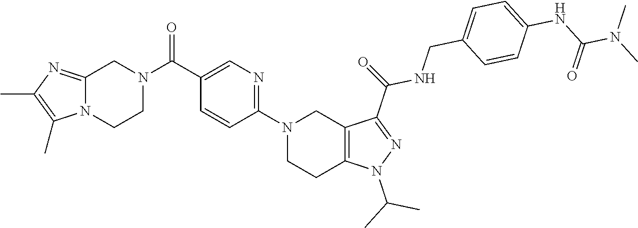 | 5-(5-(2,3-dimethyl- 5,6,7,8- tetrahydroimidazo[1,2- a]pyrazine-7- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 639.8 |
| 415 | Ex. 193 | 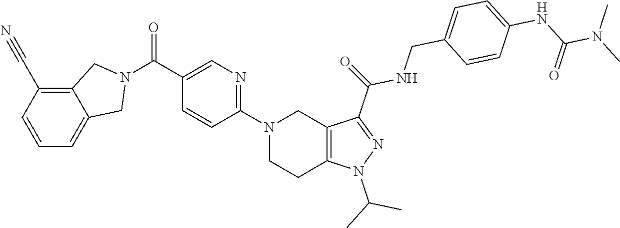 [SMILES: CC(C)n1nc(C(=O)NCc2ccc(NC(=O)N(C)C)cc2)c2c1CCN(c1ccc(C(=O)N3Cc4cccc(C#N)c4C3)cn1)C2] | 5-(5-(4- cyanoisoindoline-2- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 632.6 |
| 416 | Ex. 193 | 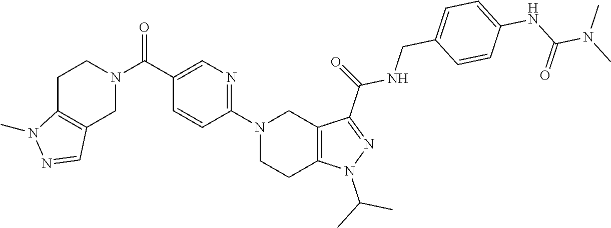 | N-(4-(3,3- dimethylureido)benzyl)- 1-isopropyl-5-(5-(1- methyl-4,5,6,7- tetrahydro-1H- pyrazolo[4,3-c]pyridine- 5-carbonyl)pyridin-2-yl)- 4,5,6,7-tetrahydro-1H- pyrazolo[4,3-c]pyridine- 3-carboxamide | 625.6 |
| 417 | Ex. 193 | 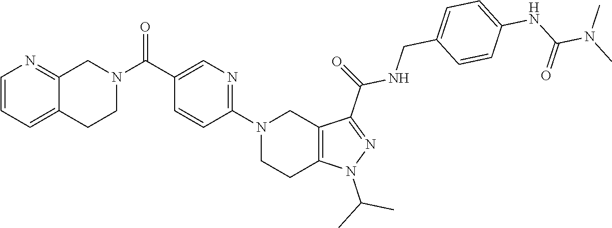 [SMILES: CC(C)n1nc(C(=O)NCc2ccc(NC(=O)N(C)C)cc2)c2c1CCN(c1ccc(C(=O)N3CCc4cccnc4C3)cn1)C2] | N-(4-(3,3- dimethylureido)benzyl)- 1-isopropyl-5-(5- (5,6,7,8-tetrahydro-1,7- naphthyridine-7- carbonyl)pyridin-2-yl)- 4,5,6,7-tetrahydro-1H- pyrazolo[4,3-c]pyridine- 3-carboxamide | 622.7 |
| 418 | Ex. 193 | 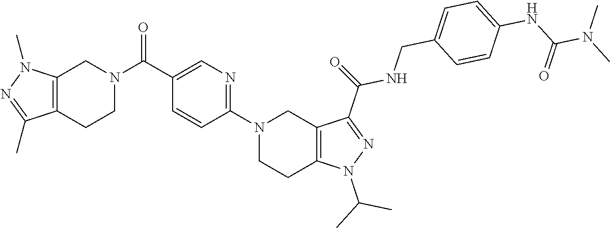 [SMILES: Cc1nn(C)c2c1CCN(C(=O)c1ccc(N3CCc4c(c(C(=O)NCc5ccc(NC(=O)N(C)C)cc5)nn4C(C)C)C3)nc1)C2] | 5-(5-(1,3-dimethyl- 4,5,6,7-tetrahydro-1H- pyrazolo[3,4-c]pyridine- 6-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 639.6 |
| 419 | Ex. 193 | 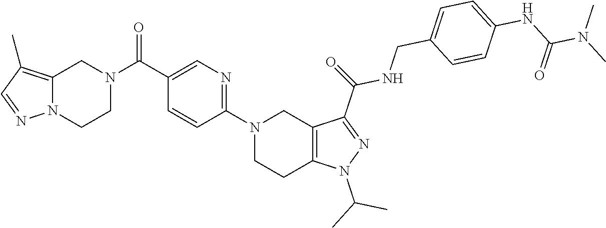 [SMILES: Cc1cnn2c1CN(C(=O)c1ccc(N3CCc4c(c(C(=O)NCc5ccc(NC(=O)N(C)C)cc5)nn4C(C)C)C3)nc1)CC2] | N-(4-(3,3- dimethylureido)benzyl)- 1-isopropyl-5-(5-(3- methyl-4,5,6,7- tetrahydropyrazolo[1,5- a]pyrazine-5- carbonyl)pyridin-2-yl)- 4,5,6,7-tetrahydro-1H- pyrazolo[4,3-c]pyridine- 3-carboxamide | 625.5 |
| 420 | Ex. 193 | 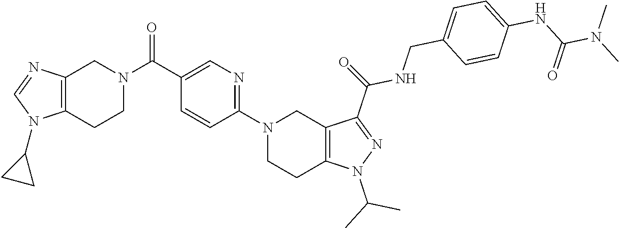 [SMILES: CC(C)n1nc(C(=O)NCc2ccc(NC(=O)N(C)C)cc2)c2c1CCN(c1ccc(C(=O)N3CCc4c(ncn4C4CC4)C3)cn1)C2] | 5-(5-(1-cyclopropyl- 4,5,6,7-tetrahydro-1H- imidazo[4,5-c]pyridine-5- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 651.8 |
| 421 | Ex. 193 | 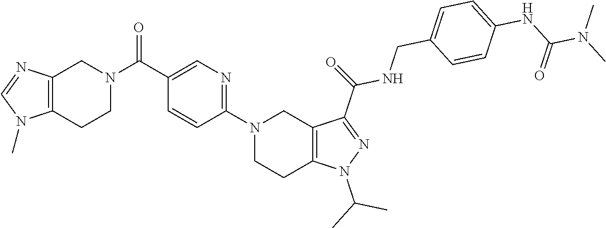 [SMILES: CC(C)n1nc(C(=O)NCc2ccc(NC(=O)N(C)C)cc2)c2c1CCN(c1ccc(C(=O)N3CCc4c(ncn4C)C3)cn1)C2] | N-(4-(3,3- dimethylureido)benzyl)- 1-isopropyl-5-(5-(1- methyl-4,5,6,7- tetrahydro-1H- imidazo[4,5-c]pyridine-5- carbonyl)pyridin-2-yl)- 4,5,6,7-tetrahydro-1H- pyrazolo[4,3-c]pyridine- 3-carboxamide | 625.7 |
| 422 | Ex. 193 | 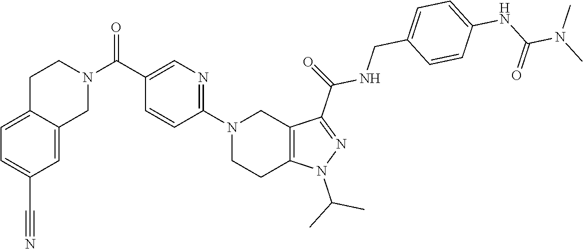 [SMILES: CC(C)n1nc(C(=O)NCc2ccc(NC(=O)N(C)C)cc2)c2c1CCN(c1ccc(C(=O)N3CCc4ccc(C#N)cc4C3)cn1)C2] | 5-(5-(7-cyano-1,2,3,4- tetrahydroisoquinoline- 2-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 646.8 |
| 423 | Ex. 193 | 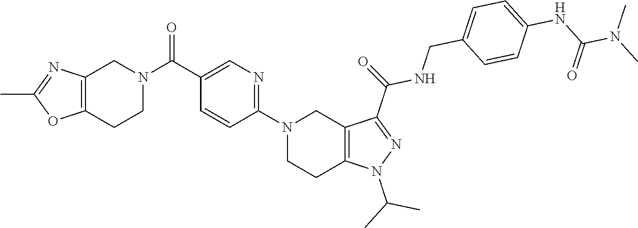 | N-(4-(3,3- dimethylureido)benzyl)- 1-isopropyl-5-(5-(2- methyl-4,5,6,7- tetrahydrooxazolo[4,5- c]pyridine-5- carbonyl)pyridin-2-yl)- 4,5,6,7-tetrahydro-1H- pyrazolo[4,3-c]pyridine- 3-carboxamide | 626.6 |
| 424 | Ex. 193 | 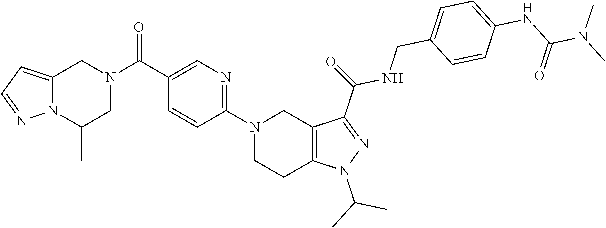 [SMILES: CC(C)n1nc(C(=O)NCc2ccc(NC(=O)N(C)C)cc2)c2c1CCN(c1ccc(C(=O)N3Cc4ccnn4C(C)C3)cn1)C2] | N-(4-(3,3- dimethylureido)benzyl)- 1-isopropyl-5-(5-(7- methyl-4,5,6,7- tetrahydropyrazolo[1,5- a]pyrazine-5- carbonyl)pyridin-2-yl)- 4,5,6,7-tetrahydro-1H- pyrazolo[4,3-c]pyridine- 3-carboxamide | 625.6 |
| 425 | Ex. 193 | 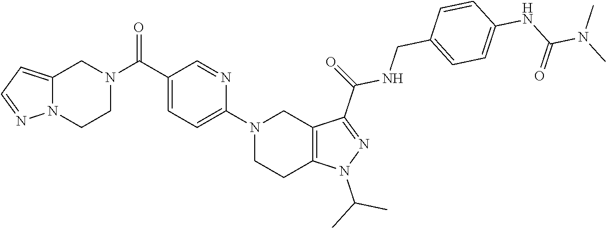 [SMILES: CC(C)n1nc(C(=O)NCc2ccc(NC(=O)N(C)C)cc2)c2c1CCN(c1ccc(C(=O)N3CCn4nccc4C3)cn1)C2] | N-(4-(3,3- dimethylureido)benzyl)- 1-isopropyl-5-(5- (4,5,6,7- tetrahydropyrazolo[1,5- a]pyrazine-5- carbonyl)pyridin-2-yl)- 4,5,6,7-tetrahydro-1H- pyrazolo[4,3-c]pyridine- 3-carboxamide | 611.5 |
| 426 | Ex. 193 | 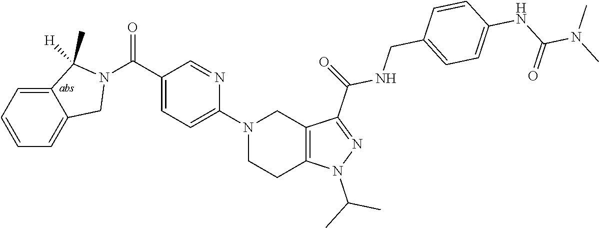 [SMILES: CC(C)n1nc(C(=O)NCc2ccc(NC(=O)N(C)C)cc2)c2c1CCN(c1ccc(C(=O)N3Cc4ccccc4[C@H]3C)cn1)C2] | (R)-N-(4-(3,3- dimethylureido)benzyl)- 1-isopropyl-5-(5-(1- methylisoindoline-2- carbonyl)pyridin-2-yl)- 4,5,6,7-tetrahydro-1H- pyrazolo[4,3-c]pyridine- 3-carboxamide | 621.5 |
| 427 | Ex. 193 | 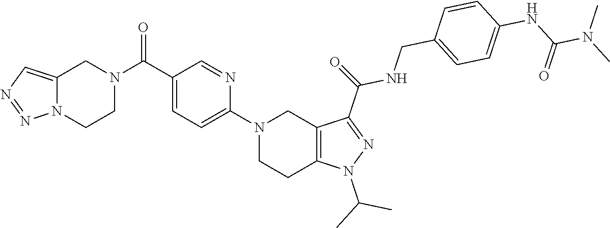 [SMILES: CC(C)n1nc(C(=O)NCc2ccc(NC(=O)N(C)C)cc2)c2c1CCN(c1ccc(C(=O)N3CCn4nncc4C3)cn1)C2] | N-(4-(3,3- dimethylureido)benzyl)- 1-isopropyl-5-(5- (4,5,6,7-tetrahydro- [1,2,3]triazolo[1,5- a]pyrazine-5- carbonyl)pyridin-2-yl)- 4,5,6,7-tetrahydro-1H- pyrazolo[4,3-c]pyridine- 3-carboxamide | 612.5 |
| 428^{∧} | Ex. 537 | 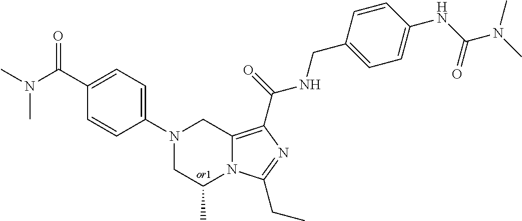 ¹H NMR (400 MHz, (CD₃)₂SO) δ 8.21 (s, 1H), 8.14-8.08 (m, 1H), 7.40-7.32 (m, 4H), 7.18-7.12 (m, 2H), 7.01-6.95 (m, 2H), 4.92 (d, 1H), 4.60-4.50 (m, 1H), 4.41- 4.28 (m, 3H), 4.04-3.95 (m, 1H), 3.43- 3.37 (m, 1H), 2.96 (s, 6H), 2.90 (s, 6H), 2.78-2.59 (m, 2H), 1.41-1.37 (m, 3H), 1.29-1.22 (m, 3H). | rel-(R or S)-7-(4- (dimethylcarbamoyl) phenyl)-N-(4-(3,3- dimethylureido)benzyl)- 3-ethyl-5-methyl-5,6,7,8- tetrahydroimidazo[1,5- a]pyrazine-1- carboxamide | 532.5 |
| 429^{∧} | Ex. 537 | 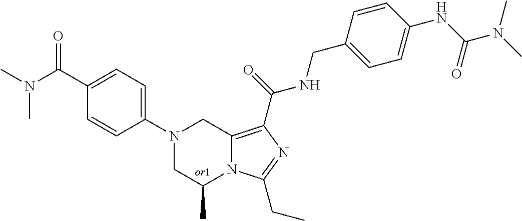 ¹H NMR (400 MHz, (CD₃)₂SO) δ 8.21 (s, 1H), 8.14-8.09 (m, 1H), 7.40-7.33 (m, 4H), 7.18-7.12 (m, 2H), 7.02-6.96 (m, 2H), 4.92 (d, 1H), 4.60-4.50 (m, 1H), 4.41- 4.29 (m, 3H), 4.03-3.95 (m, 1H), 3.44- 3.35 (m, 1H), 2.96 (s, 6H), 2.90 (s, 6H), 2.79-2.58 (m, 2H), 1.39 (d, 3H), 1.29- 1.21 (m, 3H). | rel-(R or S)-7-(4- (dimethylcarbamoyl) phenyl)-N-(4-(3,3- dimethylureido)benzyl)- 3-ethyl-5-methyl-5,6,7,8- tetrahydroimidazo[1,5- a]pyrazine-1- carboxamide | 532.7 |
| 430 | Ex. 286 | 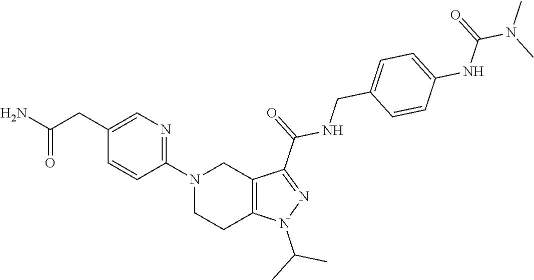 [SMILES: CC(C)n1nc(C(=O)NCc2ccc(NC(=O)N(C)C)cc2)c2c1CCN(c1ccc(CC(N)=O)cn1)C2] ¹H NMR (400 MHz, (CD₃)₂SO) δ 8.32- 8.23 (m, 1H), 8.20 (s, 1H), 7.95 (s, 1H), 7.46-7.30 (m, 4H), 7.20-7.10 (m, 2H), 6.86-6.74 (m, 2H), 4.60 (s, 2H), 4.50- 4.39 (m, 1H), 4.35-4.28 (m, 2H), 3.88- 3.74 (m, 2H), 3.23-3.16 (m, 2H), 2.88 (s, 6H), 2.79-2.72 (m, 2H), 1.36 (d, 6H). | 5-(5-(2-amino-2- oxoethyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 519.2 |
| 431 | Ex. 193 | 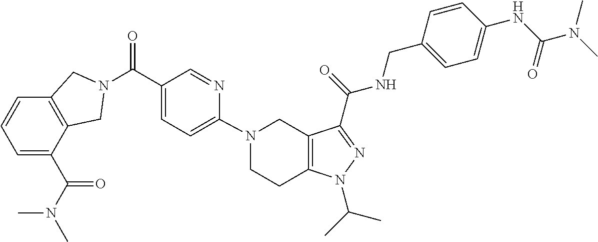 [SMILES: CC(C)n1nc(C(=O)NCc2ccc(NC(=O)N(C)C)cc2)c2c1CCN(c1ccc(C(=O)N3Cc4cccc(C(=O)N(C)C)c4C3)cn1)C2] | 5-(5-(4- (dimethylcarbamoyl) isoindoline-2- carbonyl)pyridin-2-yl)-N- (4-(3,3- dimethylureido)benzyl)- 1-isopropyl-4,5,6,7- tetrahydro-1H- pyrazolo[4,3-c]pyridine- 3-carboxamide | 678.8 |
| 432 | Ex. 193 | 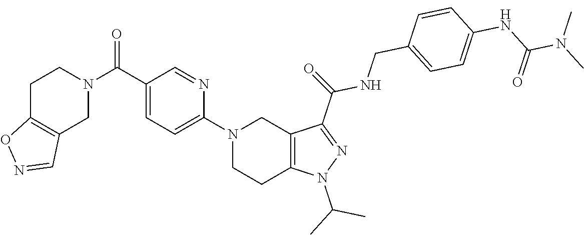 [SMILES: CC(C)n1nc(C(=O)NCc2ccc(NC(=O)N(C)C)cc2)c2c1CCN(c1ccc(C(=O)N3CCc4oncc4C3)cn1)C2] | N-(4-(3,3- dimethylureido)benzyl)- 1-isopropyl-5-(5- (4,5,6,7- tetrahydroisoxazolo[4,5- c]pyridine-5- carbonyl)pyridin-2-yl)- 4,5,6,7-tetrahydro-1H- pyrazolo[4,3-c]pyridine- 3-carboxamide | 612.8 |
| 433 | Ex. 193 | 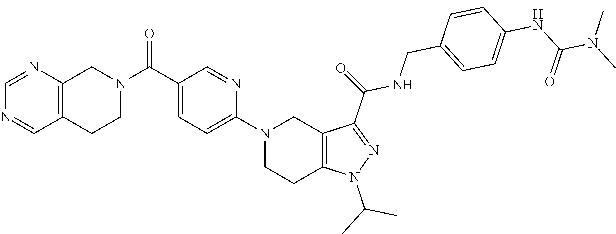 [SMILES: CC(C)n1nc(C(=O)NCc2ccc(NC(=O)N(C)C)cc2)c2c1CCN(c1ccc(C(=O)N3CCc4cncnc4C3)cn1)C2] | N-(4-(3,3- dimethylureido)benzyl)- 1-isopropyl-5-(5- (5,6,7,8- tetrahydropyrido[3,4- d]pyrimidine-7- carbonyl)pyridin-2-yl)- 4,5,6,7-tetrahydro-1H- pyrazolo[4,3-c]pyridine- 3-carboxamide | 623.6 |
| 434 | Ex. 193 | 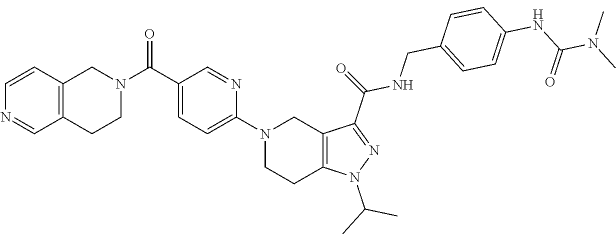 [SMILES: CC(C)n1nc(C(=O)NCc2ccc(NC(=O)N(C)C)cc2)c2c1CCN(c1ccc(C(=O)N3CCc4cnccc4C3)cn1)C2] | N-(4-(3,3- dimethylureido)benzyl)- 1-isopropyl-5-(5- (1,2,3,4-tetrahydro-2,6- naphthyridine-2- carbonyl)pyridin-2-yl)- 4,5,6,7-tetrahydro-1H- pyrazolo[4,3-c]pyridine- 3-carboxamide | 622.6 |
| 435 | Ex. 193 | 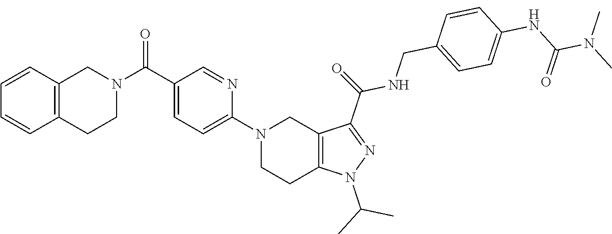 [SMILES: CC(C)n1nc(C(=O)NCc2ccc(NC(=O)N(C)C)cc2)c2c1CCN(c1ccc(C(=O)N3CCc4ccccc4C3)cn1)C2] | N-(4-(3,3- dimethylureido)benzyl)- 1-isopropyl-5-(5- (1,2,3,4- tetrahydroisoquinoline- 2-carbonyl)pyridin-2-yl)- 4,5,6,7-tetrahydro-1H- pyrazolo[4,3-c]pyridine- 3-carboxamide | 621.8 |
| 436 | Ex. 193 | 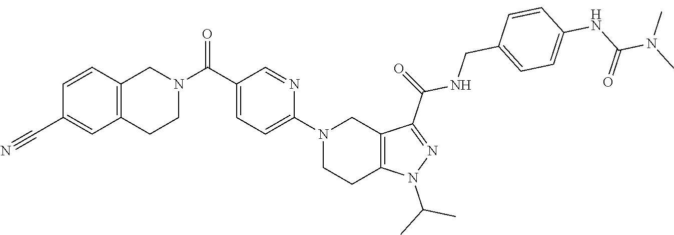 [SMILES: CC(C)n1nc(C(=O)NCc2ccc(NC(=O)N(C)C)cc2)c2c1CCN(c1ccc(C(=O)N3CCc4cc(C#N)ccc4C3)cn1)C2] | 5-(5-(6-cyano-1,2,3,4- tetrahydroisoquinoline- 2-carbonyl)pyridin-2-yl)- N-(4-(3,3- dimethylureido)benzyl)- 1-isopropyl-4,5,6,7- tetrahydro-1H- pyrazolo[4,3-c]pyridine- 3-carboxamide | 646.8 |
| 437 | Ex. 193 | 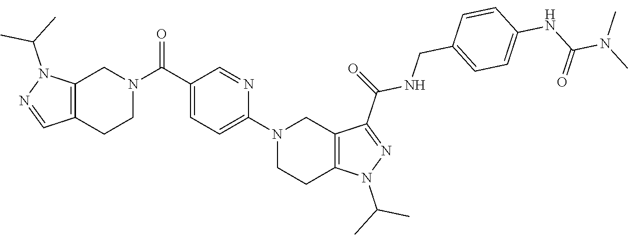 [SMILES: CC(C)n1ncc2c1CN(C(=O)c1ccc(N3CCc4c(c(C(=O)NCc5ccc(NC(=O)N(C)C)cc5)nn4C(C)C)C3)nc1)CC2] | N-(4-(3,3- dimethylureido)benzyl)- 1-isopropyl-5-(5-(1- isopropyl-4,5,6,7- tetrahydro-1H- pyrazolo[3,4-c]pyridine- 6-carbonyl)pyridin-2-yl)- 4,5,6,7-tetrahydro-1H- pyrazolo[4,3-c]pyridine- 3-carboxamide | 653.8 |
| 438 | Ex. 193 | 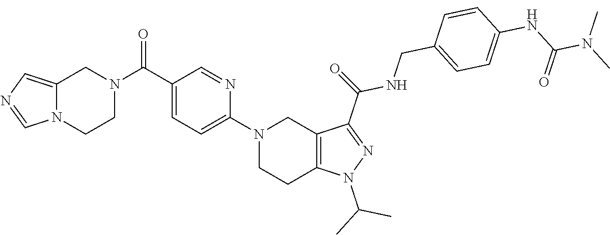 [SMILES: CC(C)n1nc(C(=O)NCc2ccc(NC(=O)N(C)C)cc2)c2c1CCN(c1ccc(C(=O)N3CCn4cncc4C3)cn1)C2] | N-(4-(3,3- dimethylureido)benzyl)- 1-isopropyl-5-(5- (5,6,7,8- tetrahydroimidazo[1,5- a]pyrazine-7- carbonyl)pyridin-2-yl)- 4,5,6,7-tetrahydro-1H- pyrazolo[4,3-c]pyridine- 3-carboxamide | 611.7 |
| 439 | Ex. 193 | 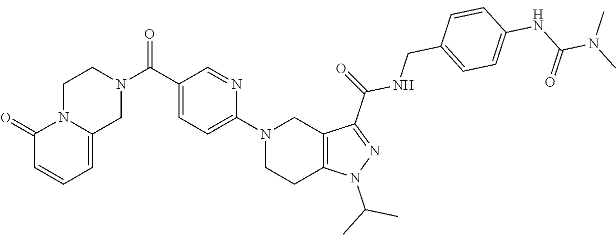 [SMILES: CC(C)n1nc(C(=O)NCc2ccc(NC(=O)N(C)C)cc2)c2c1CCN(c1ccc(C(=O)N3CCn4c(cccc4=O)C3)cn1)C2] | N-(4-(3,3- dimethylureido)benzyl)- 1-isopropyl-5-(5-(6-oxo- 1,3,4,6-tetrahydro-2H- pyrido[1,2-a]pyrazine-2- carbonyl)pyridin-2-yl)- 4,5,6,7-tetrahydro-1H- pyrazolo[4,3-c]pyridine- 3-carboxamide | 638.8 |
| 440 | Ex. 193 | 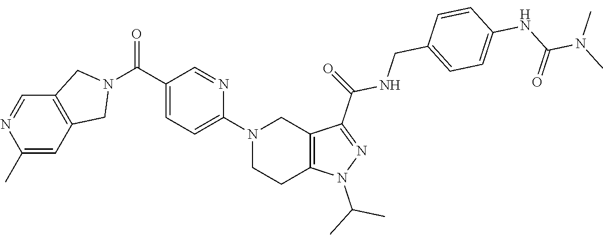 [SMILES: Cc1cc2c(cn1)CN(C(=O)c1ccc(N3CCc4c(c(C(=O)NCc5ccc(NC(=O)N(C)C)cc5)nn4C(C)C)C3)nc1)C2] | N-(4-(3,3- dimethylureido)benzyl)- 1-isopropyl-5-(5-(6- methyl-2,3-dihydro-1H- pyrrolo[3,4-c]pyridine-2- carbonyl)pyridin-2-yl)- 4,5,6,7-tetrahydro-1H- pyrazolo[4,3-c]pyridine- 3-carboxamide | 622.6 |
| 441 | Ex. 193 | 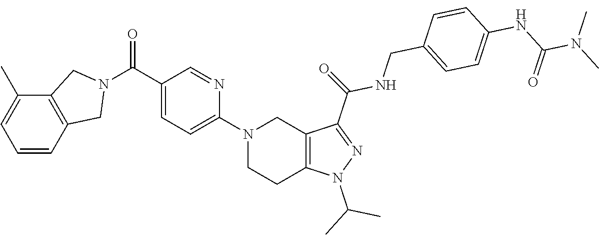 [SMILES: Cc1cccc2c1CN(C(=O)c1ccc(N3CCc4c(c(C(=O)NCc5ccc(NC(=O)N(C)C)cc5)nn4C(C)C)C3)nc1)C2] | N-(4-(3,3- dimethylureido)benzyl)- 1-isopropyl-5-(5-(4- methylisoindoline-2- carbonyl)pyridin-2-yl)- 4,5,6,7-tetrahydro-1H- pyrazolo[4,3-c]pyridine- 3-carboxamide | 621.8 |
| 442 | Ex. 193 | 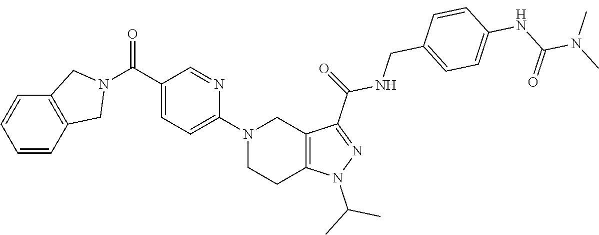 [SMILES: CC(C)n1nc(C(=O)NCc2ccc(NC(=O)N(C)C)cc2)c2c1CCN(c1ccc(C(=O)N3Cc4ccccc4C3)cn1)C2] | N-(4-(3,3- dimethylureido)benzyl)- 5-(5-(isoindoline-2- carbonyl)pyridin-2-yl)-1- isopropyl-4,5,6,7- tetrahydro-1H- pyrazolo[4,3-c]pyridine- 3-carboxamide | 607.8 |
| 443 | Ex. 193 | 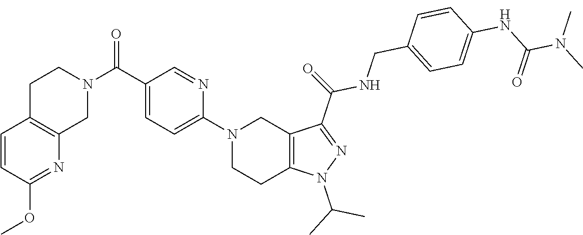 [SMILES: COc1ccc2c(n1)CN(C(=O)c1ccc(N3CCc4c(c(C(=O)NCc5ccc(NC(=O)N(C)C)cc5)nn4C(C)C)C3)nc1)CC2] | N-(4-(3,3- dimethylureido)benzyl)- 1-isopropyl-5-(5-(2- methoxy-5,6,7,8- tetrahydro-1,7- naphthyridine-7- carbonyl)pyridin-2-yl)- 4,5,6,7-tetrahydro-1H- pyrazolo[4,3-c]pyridine- 3-carboxamide | 652.7 |
| 444^{∧} | Ex. 113 | 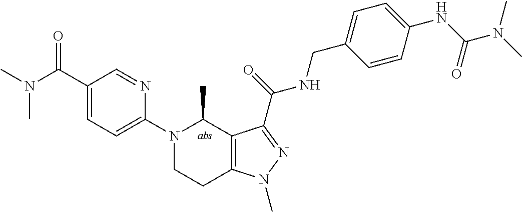 ¹H NMR (400 MHz, (CD₃)₂SO) δ 8.42- 8.36 (m, 1H), 8.20-8.13 (m, 2H), 7.60- 7.51 (m, 1H), 7.36-7.27 (m, 2H), 7.14- 7.05 (m, 2H), 6.84-6.76 (m, 1H), 5.82- 5.71 (m, 1H), 4.60-4.48 (m, 1H), 4.38- 4.18 (m, 2H), 3.73-3.64 (m, 3H), 3.40- 3.38 (m, 1H), 2.95-2.88 (m, 6H), 2.87- 2.81 (m, 6H), 2.73-2.65 (m, 2H), 1.41- 1.34 (m, 3H). | (S)-5-(5- (dimethylcarbamoyl) pyridin-2-yl)-N-(4-(3,3- dimethylureido)benzyl)- 1,4-dimethyl-4,5,6,7- tetrahydro-1H- pyrazolo[4,3-c]pyridine- 3-carboxamide | 519.3 |
| 445^{∧} | Ex. 113 | 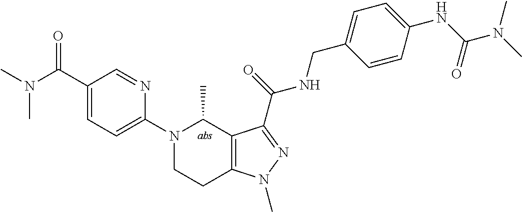 ¹H NMR (400 MHz, (CD₃)₂SO) δ 8.45- 8.41 (m, 1H), 8.23-8.20 (m, 2H), 7.61 (dd, 1H), 7.39-7.35 (m, 2H), 7.17-7.12 (m, 2H), 6.88-6.83 (m, 1H), 5.84-5.76 (m, 1H), 4.64-4.53 (m, 1H), 4.42-4.22 (m, 2H), 3.73 (s, 3H), 3.43-3.36 (m, 1H), 2.96 (s, 6H), 2.90 (s, 6H), 2.78-2.69 (m, 2H), 1.43 (d, 3H). | (R)-5-(5- (dimethylcarbamoyl) pyridin-2-yl)-N-(4-(3,3- dimethylureido)benzyl)- 1,4-dimethyl-4,5,6,7- tetrahydro-1H- pyrazolo[4,3-c]pyridine- 3-carboxamide | 519.3 |
| 446^{∧} | Ex. 23^{k} | 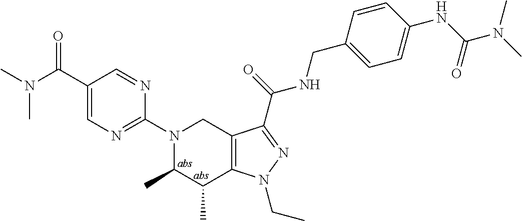 ¹H NMR (600 MHz, (CD₃)₂SO) δ 8.57- 8.50 (m, 2H), 8.49-8.42 (m, 1H), 8.25- 8.20 (m, 1H), 7.42-7.35 (m, 2H), 7.21- 7.14 (m, 2H), 5.52-5.45 (m, 1H), 5.35- 5.26 (m, 1H), 4.41-4.25 (m, 2H), 4.19- 4.06 (m, 3H), 3.10-3.04 (m, 1H), 3.03- 2.95 (m, 6H), 2.94-2.88 (m, 6H), 1.44- 1.33 (m, 3H), 1.17-1.11 (m, 3H), 1.09- 1.01 (m, 3H). | (6R,7R)-5-(5- (dimethylcarbamoyl) pyrimidin-2-yl)-N-(4-(3,3- dimethylureido)benzyl)- 1-ethyl-6,7-dimethyl- 4,5,6,7-tetrahydro-1H- pyrazolo[4,3-c]pyridine- 3-carboxamide | 548.3 |
| 447^{∧} | Ex. 23^{k} | 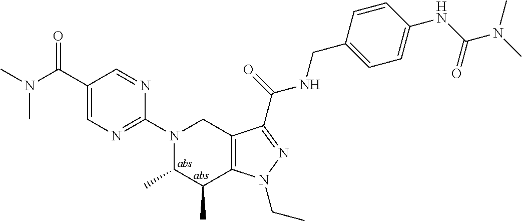 ¹H NMR (600 MHz, (CD₃)₂SO) δ 8.53 (s, 2H), 8.46 (t, 1H), 8.22 (s, 1H), 7.40-7.36 (m, 2H), 7.20-7.15 (m, 2H), 5.51-5.45 (m, 1H), 5.30 (q, 1H), 4.38-4.27 (m, 2H), 4.17-4.06 (m, 3H), 3.10-3.05 (m, 1H), 3.04-2.94 (m, 6H), 2.91-2.89 (m, 6H), 1.40-1.35 (m, 3H), 1.15-1.11 (m, 3H), 1.08-1.04 (m, 3H). | (6S,7S)-5-(5- (dimethylcarbamoyl) pyrimidin-2-yl)-N-(4-(3,3- dimethylureido)benzyl)- 1-ethyl-6,7-dimethyl- 4,5,6,7-tetrahydro-1H- pyrazolo[4,3-c]pyridine- 3-carboxamide | 548.3 |
| 448^{∧} | Ex. 4^{k} | 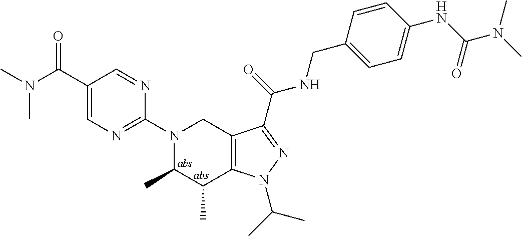 ¹H NMR (600 MHz, (CD₃)₂SO) δ 8.53 (s, 2H), 8.36 (t, 1H), 8.23 (s, 1H), 7.41-7.37 (m, 2H), 7.21-7.17 (m, 2H), 5.47 (d, 1H), 5.33-5.27 (m, 1H), 4.55-4.46 (m, 1H), 4.40-4.28 (m, 2H), 4.15 (d, 1H), 3.12- 3.05 (m, 1H), 3.04-2.94 (m, 6H), 2.91 (s, 6H), 1.45 (d, 3H), 1.40 (d, 3H), 1.13 (d, 3H), 1.06 (d, 3H). | (6R,7R)-5-(5- (dimethylcarbamoyl) pyrimidin-2-yl)-N-(4-(3,3- dimethylureido)benzyl)- 1-isopropyl-6,7- dimethyl-4,5,6,7- tetrahydro-1H- pyrazolo[4,3-c]pyridine- 3-carboxamide | 562.3 |
| 449^{∧} | Ex. 4^{k} | 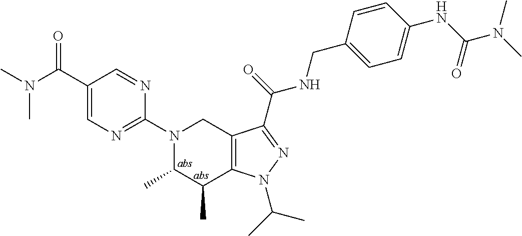 ¹H NMR (600 MHz, (CD₃)₂SO) δ 8.57- 8.50 (m, 2H), 8.36 (t, 1H), 8.23 (s, 1H), 7.43- 7.37 (m, 2H), 7.21-7.17 (m, 2H), 5.47 (d, 1H), 5.34-5.27 (m, 1H), 4.55-4.46 (m, 1H), 4.40-4.28 (m, 2H), 4.15 (d, 1H), 3.12- 3.05 (m, 1H), 3.04-2.94 (m, 6H), 2.91 (s, 6H), 1.45 (d, 3H), 1.40 (d, 3H), 1.13 (d, 3H), 1.06 (d, 3H). | (6S,7S)-5-(5- (dimethylcarbamoyl) pyrimidin-2-yl)-N-(4-(3,3- dimethylureido)benzyl)- 1-isopropyl-6,7- dimethyl-4,5,6,7- tetrahydro-1H- pyrazolo[4,3-c]pyridine- 3-carboxamide | 562.3 |
| 450 | Ex. 128 | 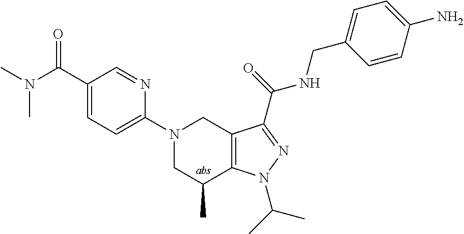 [SMILES: CC(C)n1nc(C(=O)NCc2ccc(N)cc2)c2c1[C@@H](C)CN(c1ccc(C(=O)N(C)C)cn1)C2] ¹H NMR (400 MHz, (CD₃)₂SO) δ 8.25- 8.22 (m, 1H), 8.12 (t, 1H), 7.65-7.60 (m, 1H), 7.01-6.96 (m, 2H), 6.88 (d, 1H), 6.52- 6.47 (m, 2H), 5.08 (d, 1H), 4.93 (s, 2H), 4.59-4.46 (m, 1H), 4.43-4.35 (m, 1H), 4.29-4.16 (m, 3H), 3.25-3.15 (m, 2H), 2.97 (s, 6H), 1.44-1.35 (m, 6H), 1.21- 1.15 (m, 3H). | (S)-N-(4-aminobenzyl)- 5-(5- (dimethylcarbamoyl) pyridin-2-yl)-1-isopropyl-7- methyl-4,5,6,7- tetrahydro-1H- pyrazolo[4,3-c]pyridine- 3-carboxamide | 476.3 |
| 451 | Ex. 125^{e} | 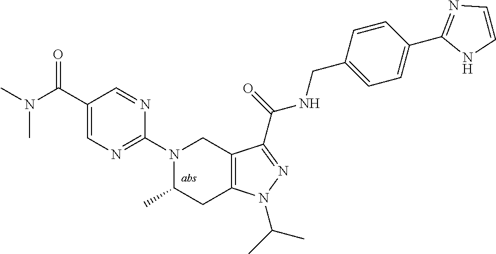 [SMILES: CC(C)n1nc(C(=O)NCc2ccc(-c3ncc[nH]3)cc2)c2c1C[C@H](C)N(c1ncc(C(=O)N(C)C)cn1)C2] ¹H NMR* (600 MHz, (CD₃)₂SO) δ 14.58 (s, 1H), 8.63 (t, 1H), 8.53-8.52 (m, 2H), 7.97- 7.92 (m, 2H), 7.80 (s, 2H), 7.60-7.55 (m, 2H), 5.63-5.55 (m, 2H), 4.56-4.46 (m, 3H), 4.14 (d, 1H), 3.04-2.92 (m, 7H), 2.82- 2.77 (m, 1H), 1.45 (d, 3H), 1.39 (d, 3H), 1.16-1.12 (m, 3H). | (S)-N-(4-(1H-imidazol-2- yl)benzyl)-5-(5- (dimethylcarbamoyl) pyrimidin-2-yl)-1-isopropyl- 6-methyl-4,5,6,7- tetrahydro-1H- pyrazolo[4,3-c]pyridine- 3-carboxamide | 528.5 |
| 452 | Ex. 125^{e} | 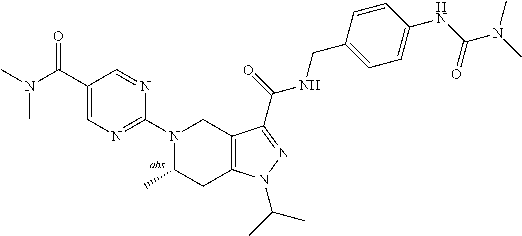 ¹H NMR (600 MHz, (CD₃)₂SO) δ 8.54- 8.52 (m, 2H), 8.34 (t, 1H), 8.23 (s, 1H), 7.42- 7.37 (m, 2H), 7.21-7.15 (m, 2H), 5.65- 5.53 (m, 2H), 4.55-4.46 (m, 1H), 4.34 (d, 2H), 4.15 (d, 1H), 3.03-2.94 (m, 6H), 2.93- 2.87 (m, 7H), 2.81-2.74 (m, 1H), 1.43 (d, 3H), 1.37 (d, 3H), 1.14 (d, 3H). | (S)-5-(5- (dimethylcarbamoyl) pyrimidin-2-yl)-N-(4-(3,3- dimethylureido)benzyl)- 1-isopropyl-6-methyl- 4,5,6,7-tetrahydro-1H- pyrazolo[4,3-c]pyridine- 3-carboxamide | 548.5 |
| 453 | Ex. 193 | 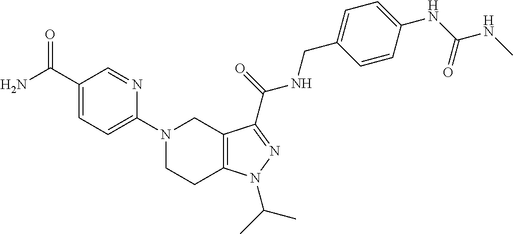 [SMILES: CNC(=O)Nc1ccc(CNC(=O)c2nn(C(C)C)c3c2CN(c2ccc(C(N)=O)cn2)CC3)cc1] ¹H NMR (400 MHz, (CD₃)₂SO) δ 8.63 (d, 1H), 8.43 (s, 1H), 8.32 (t, 1H), 7.98 (dd, 1H), 7.80-7.72 (m, 1H), 7.35-7.29 (m, 2H), 7.20-7.09 (m, 3H), 6.88 (d, 1H), 5.94 (q, 1H), 4.76 (s, 2H), 4.53-4.43 (m, 1H), 4.33 (d, 2H), 3.96 (t, 2H), 2.81 (t, 2H), 2.62 (d, 3H), 1.38 (d, 6H). | 5-(5-carbamoylpyridin-2- yl)-1-isopropyl-N-(4-(3- methylureido)benzyl)- 4,5,6,7-tetrahydro-1H- pyrazolo[4,3-c]pyridine- 3-carboxamide | 491.3 |
| 454 | Ex. 193 | 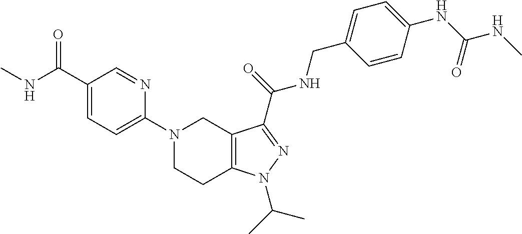 ¹H NMR (400 MHz, (CD₃)₂SO) δ 8.58 (d, 1H), 8.43 (s, 1H), 8.32 (t, 1H), 8.21 (q, 1H), 7.94 (dd, 1H), 7.34-7.27 (m, 2H), 7.20- 7.13 (m, 2H), 6.88 (d, 1H), 5.94 (q, 1H), 4.74 (s, 2H), 4.53-4.40 (m, 1H), 4.32 (d, 2H), 3.95 (t, 2H), 2.80 (t, 2H), 2.74 (d, 3H), 2.61 (d, 3H), 1.38 (d, 6H). | 1-isopropyl-5-(5- (methylcarbamoyl)pyridin- 2-yl)-N-(4-(3- methylureido)benzyl)- 4,5,6,7-tetrahydro-1H- pyrazolo[4,3-c]pyridine- 3-carboxamide | 505.2 |
| 455 | Ex. 193 | 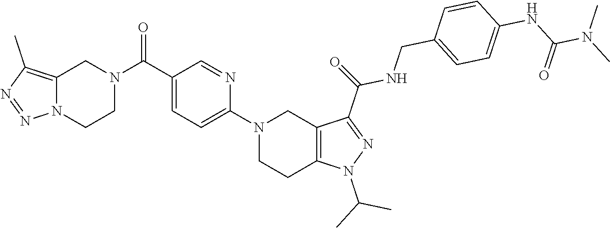 [SMILES: Cc1nnn2c1CN(C(=O)c1ccc(N3CCc4c(c(C(=O)NCc5ccc(NC(=O)N(C)C)cc5)nn4C(C)C)C3)nc1)CC2] | N-(4-(3,3- dimethylureido)benzyl)- 1-isopropyl-5-(5-(3- methyl-4,5,6,7- tetrahydro- [1,2,3]triazolo[1,5- a]pyrazine-5- carbonyl)pyridin-2-yl)- 4,5,6,7-tetrahydro-1H- pyrazolo[4,3-c]pyridine- 3-carboxamide | 626.7 |
| 456 | Ex. 193 | 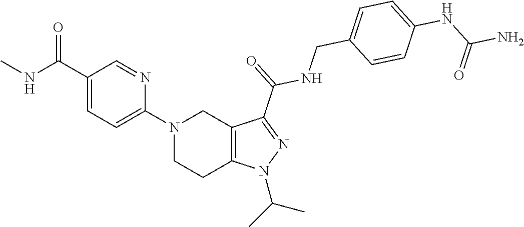 [SMILES: CNC(=O)c1ccc(N2CCc3c(c(C(=O)NCc4ccc(NC(N)=O)cc4)nn3C(C)C)C2)nc1] ¹H NMR (400 MHz, (CD₃)₂SO) δ 8.58 (d, 1H), 8.45 (s, 1H), 8.32 (t, 1H), 8.21 (q, 1H), 7.94 (dd, 1H), 7.34-7.27 (m, 2H), 7.19- 7.14 (m, 2H), 6.88 (d, 1H), 5.78 (s, 2H), 4.74 (s, 2H), 4.51-4.41 (m, 1H), 4.32 (d, 2H), 3.95 (t, 2H), 2.80 (t, 2H), 2.73 (d, 3H), 1.40-1.32 (m, 6H). | 1-isopropyl-5-(5- (methylcarbamoyl)pyridin- 2-yl)-N-(4- ureidobenzyl)-4,5,6,7- tetrahydro-1H- pyrazolo[4,3-c]pyridine- 3-carboxamide | 491.3 |
| 457 | Ex. 193 | 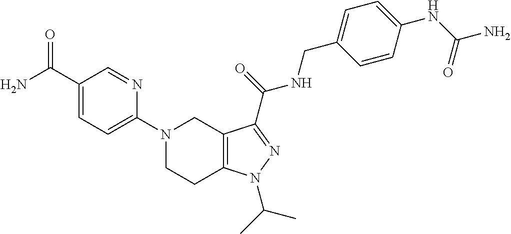 [SMILES: CC(C)n1nc(C(=O)NCc2ccc(NC(N)=O)cc2)c2c1CCN(c1ccc(C(N)=O)cn1)C2] ¹H NMR (400 MHz, (CD₃)₂SO) δ 8.62 (d, 1H), 8.46 (s, 1H), 8.31 (t, 1H), 7.97 (dd, 1H), 7.79-7.72 (m, 1H), 7.34-7.28 (m, 2H), 7.19-7.09 (m, 3H), 6.88 (d, 1H), 5.78 (s, 2H), 4.75 (s, 2H), 4.54-4.41 (m, 1H), 4.32 (d, 2H), 3.96 (t, 2H), 2.80 (t, 2H), 1.38 (d, 6H). | 5-(5-carbamoylpyridin-2- yl)-1-isopropyl-N-(4- ureidobenzyl)-4,5,6,7- tetrahydro-1H- pyrazolo[4,3-c]pyridine- 3-carboxamide | 477.1 |
| 458 | Ex. 127 | 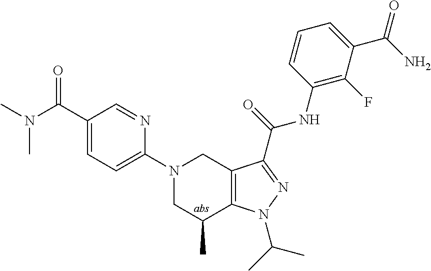 [SMILES: CC(C)n1nc(C(=O)Nc2cccc(C(N)=O)c2F)c2c1[C@@H](C)CN(c1ccc(C(=O)N(C)C)cn1)C2] ¹H NMR (400 MHz, (CD₃)₂SO) δ 9.42 (s, 1H), 8.24 (d, 1H), 8.08-8.02 (m, 1H), 7.78 (s, 1H), 7.68-7.60 (m, 2H), 7.45-7.38 (m, 1H), 7.26 (t, 1H), 6.92 (d, 1H), 5.15 (d, 1H), 4.66-4.54 (m, 1H), 4.47-4.39 (m, 1H), 4.28 (d, 1H), 3.41-3.36 (m, 2H), 2.97 (s, 6H), 1.50-1.42 (m, 6H), 1.22 (d, 3H). | (S)-N-(3-carbamoyl-2- fluorophenyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 508.2 |
| 459 | Ex. 530 | 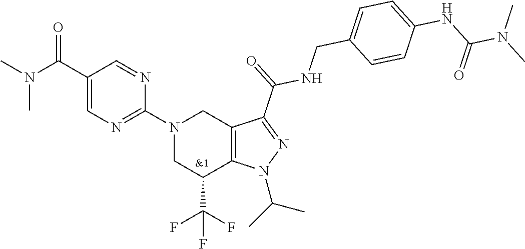 ¹H NMR (400 MHz, (CD₃)₂SO) δ 8.52- 8.44 (m, 3H), 8.17 (s, 1H), 7.36-7.30 (m, 2H), 7.16-7.08 (m, 2H), 5.51-5.39 (m, 2H), 4.65-4.54 (m, 1H), 4.50-4.41 (m, 1H), 4.36-4.19 (m, 3H), 3.26-3.24 (m, 1H), 2.93 (s, 6H), 2.84 (s, 6H), 1.36 (dd, 6H). | rac-(R)-5-(5- (dimethylcarbamoyl) pyrimidin-2-yl)-N-(4-(3,3- dimethylureido)benzyl)- 1-isopropyl-7- (trifluoromethyl)-4,5,6,7- tetrahydro-1H- pyrazolo[4,3-c]pyridine- 3-carboxamide | 602.3 |
| 460 | Ex. 530 | 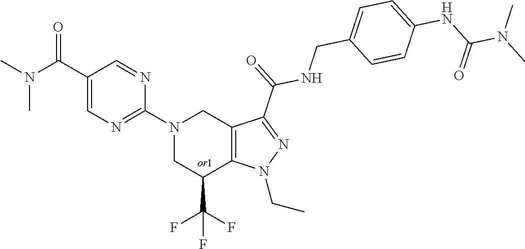 ¹H NMR (400 MHz, CDCl₃) δ 8.52 (s, 2H), 7.37-7.33 (m, 2H), 7.31-7.28 (m, 2H), 7.10 (t, 1H), 6.31 (s, 1H), 5.87-5.67 (m, 2H), 4.64-4.42 (m, 3H), 4.12 (q, 2H), 3.68- 3.56 (m, 1H), 3.35-3.25 (m, 1H), 3.10 (s, 6H), 3.03 (s, 6H), 1.47 (t, 3H). | rac-(R)-5-(5- (dimethylcarbamoyl) pyrimidin-2-yl)-N-(4-(3,3- dimethylureido)benzyl)- 1-ethyl-7- (trifluoromethyl)-4,5,6,7- tetrahydro-1H- pyrazolo[4,3-c]pyridine- 3-carboxamide | 588.3 |
| 461^{∧} | Ex. 530 | 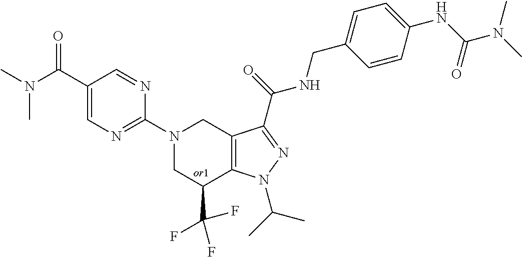 ¹H NMR (400 MHz, (CD₃)₂SO) δ 8.56- 8.48 (m, 3H), 8.22 (s, 1H), 7.40-7.34 (m, 2H), 7.19-7.13 (m, 2H), 5.56-5.43 (m, 2H), 4.70-4.59 (m, 1H), 4.57-4.47 (m, 1H), 4.40-4.25 (m, 3H), 3.45-3.37 (m, 1H), 2.98 (s, 6H), 2.88 (s, 6H), 1.41 (dd, 6H). | rel-(R or S)-5-(5- (dimethylcarbamoyl) pyrimidin-2-yl)-N-(4-(3,3- dimethylureido)benzyl)- 1-isopropyl-7- (trifluoromethyl)-4,5,6,7- tetrahydro-1H- pyrazolo[4,3-c]pyridine- 3-carboxamide | 602.3 |
| 462^{∧} | Ex. 530 | 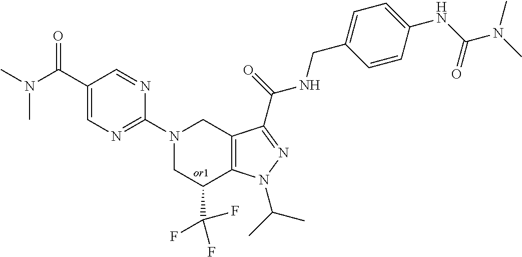 ¹H NMR (400 MHz, (CD₃)₂SO) δ 8.57- 8.48 (m, 3H), 8.22 (s, 1H), 7.41-7.34 (m, 2H), 7.20-7.13 (m, 2H), 5.57-5.42 (m, 2H), 4.69-4.59 (m, 1H), 4.56-4.47 (m, 1H), 4.39-4.25 (m, 3H), 3.43-3.39 (m, 1H), 2.98 (s, 6H), 2.89 (s, 6H), 1.41 (dd, 6H). | rel-(R or S)-5-(5- (dimethylcarbamoyl) pyrimidin-2-yl)-N-(4-(3,3- dimethylureido)benzyl)- 1-isopropyl-7- (trifluoromethyl)-4,5,6,7- tetrahydro-1H- pyrazolo[4,3-c]pyridine- 3-carboxamide | 602.3 |
| 463 | Ex. 39^{l} | 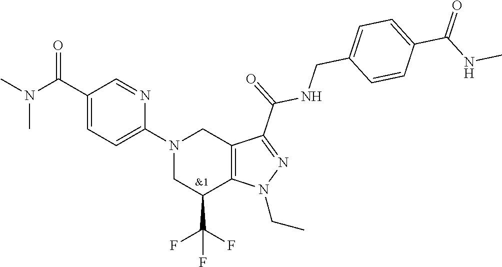 ¹H NMR (400 MHz, (CD₃)₂SO) δ 8.84- 8.78 (m, 1H), 8.40-8.34 (m, 1H), 8.27 (d, 1H), 7.80-7.76 (m, 2H), 7.68 (dd, 1H), 7.41-7.36 (m, 2H), 6.84 (d, 1H), 5.32- 5.03 (m, 2H), 4.54-4.44 (m, 3H), 4.31- 4.15 (m, 3H), 3.29-3.23 (m, 1H), 2.98 (s, 6H), 2.77 (d, 3H), 1.43 (t, 3H). | rac-(R)-5-(5- (dimethylcarbamoyl) pyridin-2-yl)-1-ethyl-N-(4- (methylcarbamoyl)benzyl)- 7-(trifluoromethyl)- 4,5,6,7-tetrahydro-1H- pyrazolo[4,3-c]pyridine- 3-carboxamide | 558.2 |
| 464^{∧} | Ex. 530 | 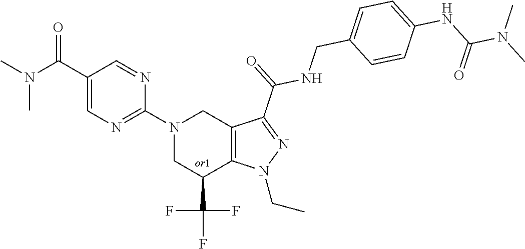 ¹H NMR (400 MHz, (CD₃)₂SO) δ 8.64- 8.59 (m, 1H), 8.57 (s, 2H), 8.23 (s, 1H), 7.43-7.37 (m, 2H), 7.21-7.16 (m, 2H), 5.61-5.45 (m, 2H), 4.55-4.45 (m, 1H), 4.41-4.28 (m, 3H), 4.22-4.14 (m, 2H), 3.43-3.38 (m, 1H), 3.00 (s, 6H), 2.91 (s, 6H), 1.42 (t, 3H). | rel-(R or S)-5-(5- (dimethylcarbamoyl) pyrimidin-2-yl)-N-(4-(3,3- dimethylureido)benzyl)- 1-ethyl-7- (trifluoromethyl)-4,5,6,7- tetrahydro-1H- pyrazolo[4,3-c]pyridine- 3-carboxamide | 588.3 |
| 465^{∧} | Ex. 530 | 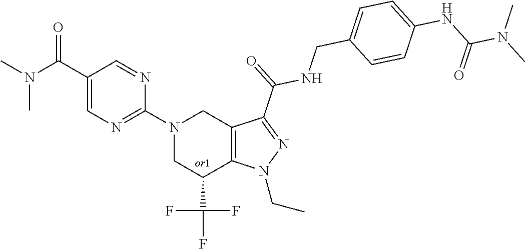 ¹H NMR (400 MHz, (CD₃)₂SO) δ 8.64- 8.59 (m, 1H), 8.57 (s, 2H), 8.23 (s, 1H), 7.43-7.36 (m, 2H), 7.20-7.15 (m, 2H), 5.62-5.44 (m, 2H), 4.54-4.44 (m, 1H), 4.41-4.28 (m, 3H), 4.23-4.14 (m, 2H), 3.44-3.37 (m, 1H), 3.00 (s, 6H), 2.91 (s, 6H), 1.42 (t, 3H). | rel-(R or S)-5-(5- (dimethylcarbamoyl) pyrimidin-2-yl)-N-(4-(3,3- dimethylureido)benzyl)- 1-ethyl-7- (trifluoromethyl)-4,5,6,7- tetrahydro-1H- pyrazolo[4,3-c]pyridine- 3-carboxamide | 588.3 |
| 466 | Ex. 127 | 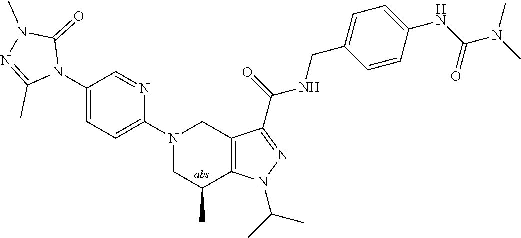 [SMILES: Cc1nn(C)c(=O)n1-c1ccc(N2Cc3c(C(=O)NCc4ccc(NC(=O)N(C)C)cc4)nn(C(C)C)c3[C@@H](C)C2)nc1] ¹H NMR (400 MHz, (CD₃)₂SO) δ 8.35 (t, 1H), 8.23 (s, 1H), 8.12 (d, 1H), 7.59 (dd, 1H), 7.42-7.35 (m, 2H), 7.21-7.15 (m, 2H), 7.00 (d, 1H), 5.06 (d, 1H), 4.60-4.49 (m, 1H), 4.45-4.21 (m, 4H), 3.39-3.35 (m, 1H), 3.32-3.31 (m, 3H), 3.28-3.19 (m, 1H), 2.90 (s, 6H), 2.04 (s, 3H), 1.43 (dd, 6H), 1.22 (d, 3H). | (S)-5-(5-(1,3-dimethyl-5- oxo-1,5-dihydro-4H- 1,2,4-triazol-4-yl)pyridin- 2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 587.3 |
| 467^{∧} | Ex. 39^{l} | 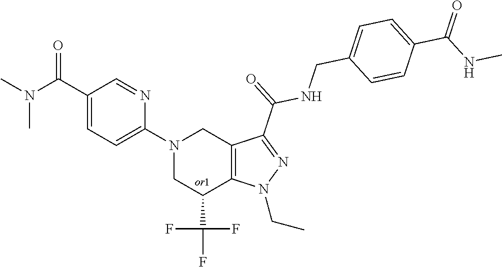 ¹H NMR (400 MHz, (CD₃)₂SO) δ 8.81 (t, 1H), 8.40-8.34 (m, 1H), 8.27 (d, 1H), 7.80- 7.74 (m, 2H), 7.68 (dd, 1H), 7.41-7.35 (m, 2H), 6.83 (d, 1H), 5.31-5.00 (m, 2H), 4.54-4.41 (m, 3H), 4.32-4.14 (m, 3H), 3.30-3.24 (m, 1H), 2.98 (s, 6H), 2.76 (d, 3H), 1.42 (t, 3H). | rel-(R or S)-5-(5- (dimethylcarbamoyl) pyridin-2-yl)-1-ethyl-N-(4- (methylcarbamoyl)benzyl)- 7-(trifluoromethyl)- 4,5,6,7-tetrahydro-1H- pyrazolo[4,3-c]pyridine- 3-carboxamide | 558.2 |
| 468^{∧} | Ex. 39^{l} | 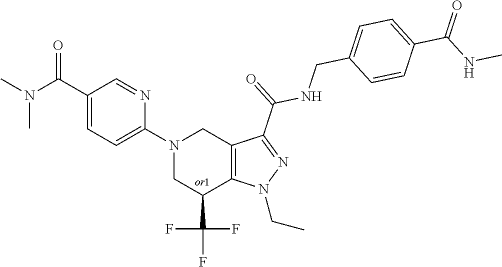 ¹H NMR (400 MHz, (CD₃)₂SO) δ 8.81 (t, 1H), 8.40-8.34 (m, 1H), 8.27 (d, 1H), 7.80- 7.74 (m, 2H), 7.68 (dd, 1H), 7.40-7.35 (m, 2H), 6.83 (d, 1H), 5.31-5.00 (m, 2H), 4.55-4.41 (m, 3H), 4.30-4.12 (m, 3H), 3.29-3.25 (m, 1H), 2.98 (s, 6H), 2.76 (d, 3H), 1.42 (t, 3H). | rel-(R or S)-5-(5- (dimethylcarbamoyl) pyridin-2-yl)-1-ethyl-N-(4- (methylcarbamoyl)benzyl)- 7-(trifluoromethyl)- 4,5,6,7-tetrahydro-1H- pyrazolo[4,3-c]pyridine- 3-carboxamide | 558.3 |
| 469 | Ex. 469 | 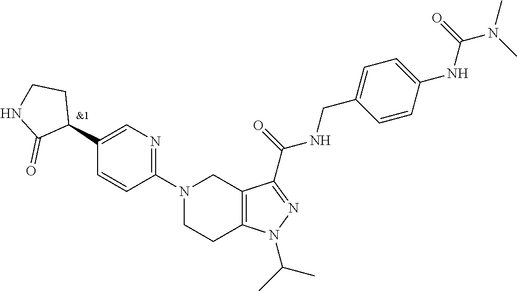 [SMILES: CC(C)n1nc(C(=O)NCc2ccc(NC(=O)N(C)C)cc2)c2c1CCN(c1ccc([C@@H]3CCNC3=O)cn1)C2] ¹H NMR (400 MHz, CDCl₃) δ 8.07 (d, 1H), 7.40 (dd, 1H), 7.37-7.33 (m, 2H), 7.31- 7.27 (m, 2H), 7.16 (t, 1H), 6.79 (d, 1H), 6.32 (s, 1H), 4.70 (s, 2H), 4.55 (d, 2H), 4.42- 4.31 (m, 1H), 4.08 (t, 2H), 3.93-3.76 (m, 2H), 3.12-3.00 (m, 7H), 2.86-2.75 (m, 3H), 2.71-2.60 (m, 1H), 1.44 (d, 6H). | rac-(R)-N-(4-(3,3- dimethylureido)benzyl)- 1-isopropyl-5-(5-(2- oxopyrrolidin-3- yl)pyridin-2-yl)-4,5,6,7- tetrahydro-1H- pyrazolo[4,3-c]pyridine- 3-carboxamide | 545.3 |
| 470 | Ex. 128 | 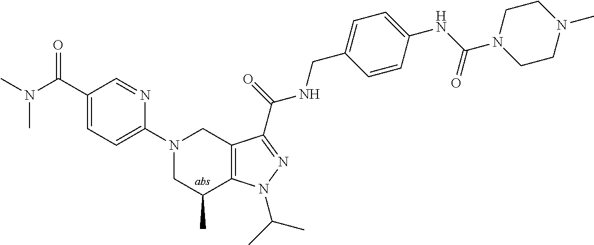 ¹H NMR (600 MHz, (CD₃)₂SO) δ 8.47 (s, 1H), 8.38-8.34 (m, 1H), 8.26-8.23 (m, 1H), 7.66-7.63 (m, 1H), 7.41-7.37 (m, 2H), 7.21-7.17 (m, 2H), 6.92-6.88 (m, 1H), 5.12-5.08 (m, 1H), 4.58-4.51 (m, 1H), 4.44-4.30 (m, 3H), 4.27-4.22 (m, 1H), 3.42 (s, 4H), 3.37-3.35 (m, 1H), 3.27- 3.21 (m, 1H), 2.99 (s, 6H), 2.31 (s, 4H), 2.20 (s, 3H), 1.45-1.40 (m, 6H), 1.22- 1.18 (m, 3H). | (S)-5-(5- (dimethylcarbamoyl) pyridin-2-yl)-1-isopropyl-7- methyl-N-(4-(4- methylpiperazine-1- carboxamido)benzyl)- 4,5,6,7-tetrahydro-1H- pyrazolo[4,3-c]pyridine- 3-carboxamide | 602.6 |
| 471 | Ex. 127 | 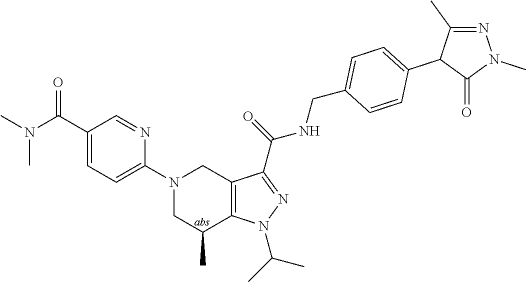 ¹H NMR (400 MHz, (CD₃)₂SO) δ 8.57 (t, 1H), 8.20 (d, 1H), 7.60 (dd, 1H), 7.45-7.40 (m, 2H), 7.34-7.29 (m, 2H), 6.85 (d, 1H), 5.06 (d, 1H), 4.57-4.35 (m, 4H), 4.20 (d, 1H), 3.30-3.27 (m, 4H), 3.23-3.16 (m, 1H), 2.94 (s, 6H), 2.01 (s, 3H), 1.40 (dd, 6H), 1.16 (d, 3H). | (S)-N-(4-(1,3-dimethyl-5- oxo-1,5-dihydro-4H- 1,2,4-triazol-4- yl)benzyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 572.3 |
| 472 | Ex. 113 | 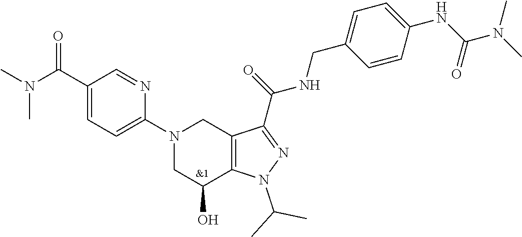 ¹H NMR (400 MHz, (CD₃)₂SO) δ 8.39 (t, 1H), 8.23 (s, 2H), 7.63 (dd, 1H), 7.43-7.35 (m, 2H), 7.21-7.14 (m, 2H), 6.89 (d, 1H), 5.67 (d, 1H), 5.08 (d, 1H), 4.94-4.88 (m, 1H), 4.78-4.67 (m, 1H), 4.46-4.31 (m, 3H), 4.14 (dd, 1H), 3.70 (dd, 1H), 2.98 (s, 6H), 2.91 (s, 6H), 1.43 (d, 6H). | rac-(R)-5-(5- (dimethylcarbamoyl) pyridin-2-yl)-N-(4-(3,3- dimethylureido)benzyl)- 7-hydroxy-1-isopropyl- 4,5,6,7-tetrahydro-1H- pyrazolo[4,3-c]pyridine- 3-carboxamide | 549.3 |
| 473 | Ex. 537 | 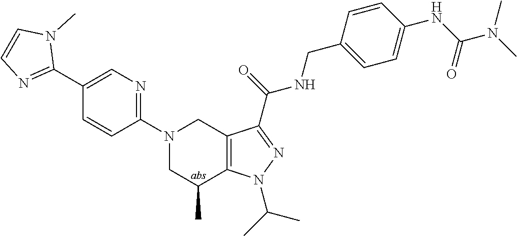 [SMILES: CC(C)n1nc(C(=O)NCc2ccc(NC(=O)N(C)C)cc2)c2c1[C@@H](C)CN(c1ccc(-c3nccn3C)cn1)C2] ¹H NMR (400 MHz, (CD₃)₂SO) δ 8.43 (d, 1H), 8.35 (t, 1H), 8.23 (s, 1H), 7.84 (dd, 1H), 7.41-7.36 (m, 1H), 7.21-7.15 (m, 3H), 6.99-6.90 (m, 2H), 5.08 (d, 1H), 4.59- 4.18 (m, 6H), 3.71 (s, 3H), 3.40-3.34 (m, 1H), 3.26-3.19 (m, 1H), 2.90 (m, 6H), 1.42 (dd, 6H), 1.22 (d, 3H). | 5-(5- (dimethylcarbamoyl) pyridin-2-yl)-N-(4-(3,3- dimethylureido)benzyl)- 7-hydroxy-1-isopropyl- 4,5,6,7-tetrahydro-1H- pyrazolo[4,3-c]pyridine- 3-carboxamide | 556.3 |
| 474^{∧} | Ex. 113 | 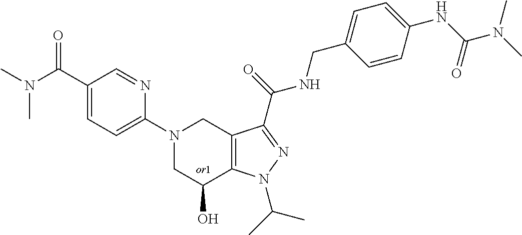 ¹H NMR (400 MHz, (CD₃)₂SO) δ 8.37 (t, 1H), 8.23-8.18 (m, 2H), 7.60 (dd, 1H), 7.38-7.33 (m, 2H), 7.17-7.12 (m, 2H), 6.86 (d, 1H), 5.65 (d, 1H), 5.05 (d, 1H), 4.90- 4.85 (m, 1H), 4.76-4.64 (m, 1H), 4.42- 4.29 (m, 3H), 4.11 (dd, 1H), 3.66 (dd, 1H), 2.94 (s, 6H), 2.88 (s, 6H), 1.40 (d, 6H). | rel-(R or S)-5-(5- (dimethylcarbamoyl) pyridin-2-yl)-N-(4-(3,3- dimethylureido)benzyl)- 7-hydroxy-1-isopropyl- 4,5,6,7-tetrahydro-1H- pyrazolo[4,3-c]pyridine- 3-carboxamide | 549.5 |
| 475^{∧} | Ex. 113 | 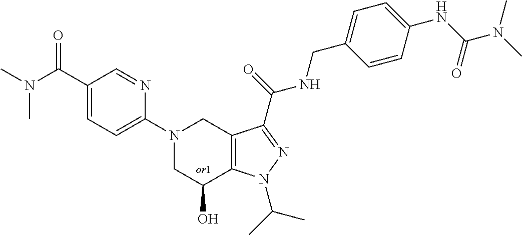 ¹H NMR (400 MHz, (CD₃)₂SO) δ 8.37 (t, 1H), 8.24-8.19 (m, 2H), 7.60 (dd, 1H), 7.39-7.33 (m, 2H), 7.17-7.11 (m, 2H), 6.86 (d, 1H), 5.71-5.59 (m, 1H), 5.06 (d, 1H), 4.92-4.83 (m, 1H), 4.75-4.64 (m, 1H), 4.44-4.29 (m, 3H), 4.12 (dd, 1H), 3.66 (dd, 1H), 2.95 (s, 6H), 2.88 (s, 6H), 1.40 (d, 6H). | rel-(R or S)-5-(5- (dimethylcarbamoyl) pyridin-2-yl)-N-(4-(3,3- dimethylureido)benzyl)- 7-hydroxy-1-isopropyl- 4,5,6,7-tetrahydro-1H- pyrazolo[4,3-c]pyridine- 3-carboxamide | 549.5 |
| 476 | Ex. 77 | 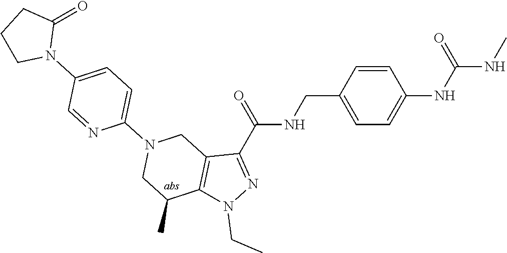 [SMILES: CCn1nc(C(=O)NCc2ccc(NC(=O)NC)cc2)c2c1[C@@H](C)CN(c1ccc(N3CCCC3=O)cn1)C2] ¹H NMR (400 MHz, (CD₃)₂SO) δ 8.44- 8.38 (m, 2H), 8.27 (d, 1H), 7.87 (dd, 1H), 7.32-7.27 (m, 2H), 7.17-7.13 (m, 2H), 6.88 (d, 1H), 5.94 (q, 1H), 4.88 (d, 1H), 4.39- 4.02 (m, 6H), 3.82-3.72 (m, 2H), 3.42- 3.36 (m, 1H), 3.21-3.13 (m, 1H), 2.61 (d, 3H), 2.42 (t, 2H), 2.10-1.99 (m, 2H), 1.37 (t, 3H), 1.20 (d, 3H). | (S)-1-ethyl-7-methyl-N- (4-(3- methylureido)benzyl)-5- (5-(2-oxopyrrolidin-1- yl)pyridin-2-yl)-4,5,6,7- tetrahydro-1H- pyrazolo[4,3-c]pyridine- 3-carboxamide | 531.5 |
| 477 | Ex. 127 | 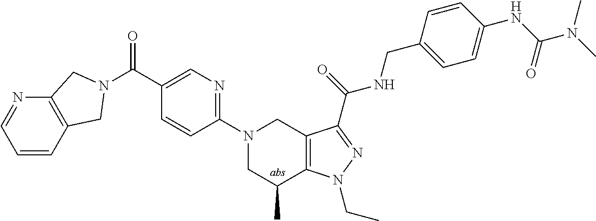 [SMILES: CCn1nc(C(=O)NCc2ccc(NC(=O)N(C)C)cc2)c2c1[C@@H](C)CN(c1ccc(C(=O)N3Cc4cccnc4C3)cn1)C2] ¹H NMR (400 MHz, (CD₃)₂SO) δ 8.53- 8.41 (m, 3H), 8.23 (s, 1H), 7.93-7.68 (m, 2H), 7.41-7.27 (m, 3H), 7.20-7.12 (m, 2H), 6.93 (d, 1H), 5.10 (d, 1H), 5.01-4.78 (m, 4H), 4.43-4.25 (m, 4H), 4.18-4.04 (m, 2H), 3.49-3.37 (m, 1H), 3.27-3.19 (m, 1H), 2.90 (s, 6H), 1.39 (t, 3H), 1.22 (d, 3H). | (S)-5-(5-(6,7-dihydro- 5H-pyrrolo[3,4- b]pyridine-6- carbonyl)pyridin-2-yl)-N- (4-(3,3- dimethylureido)benzyl)- 1-ethyl-7-methyl-4,5,6,7- tetrahydro-1H- pyrazolo[4,3-c]pyridine- 3-carboxamide | 608.3 |
| 478 | Ex. 530 | 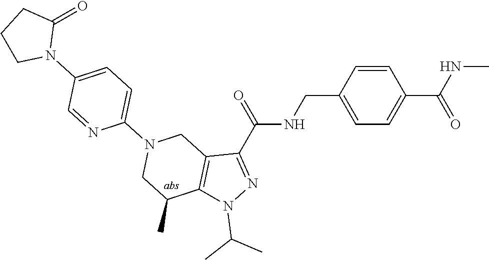 [SMILES: CNC(=O)c1ccc(CNC(=O)c2nn(C(C)C)c3c2CN(c2ccc(N4CCCC4=O)cn2)C[C@@H]3C)cc1] ¹H NMR (400 MHz, (CD₃)₂SO) δ 8.53 (t, 1H), 8.40-8.33 (m, 1H), 8.30-8.24 (m, 1H), 7.87 (dd, 1H), 7.80-7.74 (m, 2H), 7.41-7.34 (m, 2H), 6.89 (d, 1H), 4.94 (d, 1H), 4.59-4.28 (m, 4H), 4.16 (d, 1H), 3.81- 3.73 (m, 2H), 3.26-3.16 (m, 2H), 2.77 (d, 3H), 2.46-2.39 (m, 2H), 2.11-1.99 (m, 2H), 1.43 (dd, 6H), 1.21 (d, 3H). | (S)-1-isopropyl-7- methyl-N-(4- (methylcarbamoyl)benzyl)- 5-(5-(2-oxopyrrolidin- 1-yl)pyridin-2-yl)-4,5,6,7- tetrahydro-1H- pyrazolo[4,3-c]pyridine- 3-carboxamide | 530.4 |
| 479 | 39^{l} | 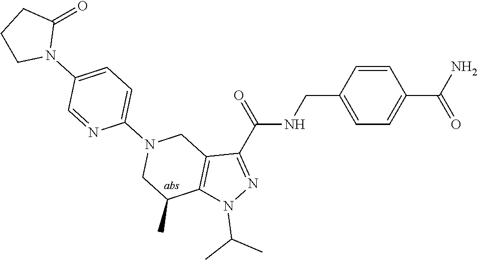 [SMILES: CC(C)n1nc(C(=O)NCc2ccc(C(N)=O)cc2)c2c1[C@@H](C)CN(c1ccc(N3CCCC3=O)cn1)C2] ¹H NMR (400 MHz, (CD₃)₂SO) δ 8.53 (t, 1H), 8.28 (d, 1H), 7.94-7.79 (m, 4H), 7.41- 7.28 (m, 3H), 6.89 (d, 1H), 4.94 (d, 1H), 4.60-4.25 (m, 4H), 4.16 (d, 1H), 3.77 (t, 2H), 3.27-3.16 (m, 2H), 2.46-2.38 (m, 2H), 2.10-2.01 (m, 2H), 1.43 (dd, 6H), 1.21 (d, 3H). | (S)-N-(4- carbamoylbenzyl)-1- isopropyl-7-methyl-5-(5- (2-oxopyrrolidin-1- yl)pyridin-2-yl)-4,5,6,7- tetrahydro-1H- pyrazolo[4,3-c]pyridine- 3-carboxamide | 516.5 |
| 480 | Ex. 39^{l} | 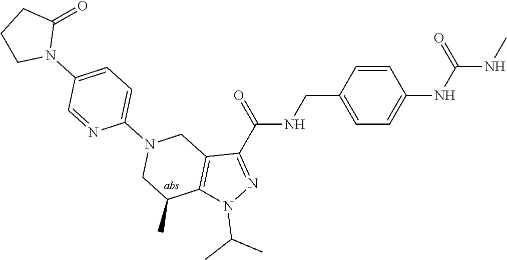 [SMILES: CNC(=O)Nc1ccc(CNC(=O)c2nn(C(C)C)c3c2CN(c2ccc(N4CCCC4=O)cn2)C[C@@H]3C)cc1] ¹H NMR (400 MHz, (CD₃)₂SO) δ 8.44 (s, 1H), 8.35-8.27 (m, 2H), 7.88 (dd, 1H), 7.36-7.28 (m, 2H), 7.20-7.13 (m, 2H), 6.89 (d, 1H), 5.95 (q, 1H), 4.94 (d, 1H), 4.60- 4.46 (m, 1H), 4.37-4.25 (m, 3H), 4.16 (d, 1H), 3.85-3.73 (m, 2H), 3.28-3.16 (m, 2H), 2.62 (d, 3H), 2.47-2.39 (m, 2H), 2.12- 2.00 (m, 2H), 1.42 (dd, 6H), 1.20 (d, 3H). | (S)-1-isopropyl-7- methyl-N-(4-(3- methylureido)benzyl)-5- (5-(2-oxopyrrolidin-1- yl)pyridin-2-yl)-4,5,6,7- tetrahydro-1H- pyrazolo[4,3-c]pyridine- 3-carboxamide | 545.4 |
| 481 | Ex. 127 | 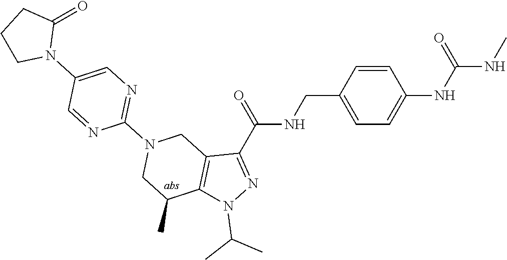 [SMILES: CNC(=O)Nc1ccc(CNC(=O)c2nn(C(C)C)c3c2CN(c2ncc(N4CCCC4=O)cn2)C[C@@H]3C)cc1] ¹H NMR (400 MHz, (CD₃)₂SO) δ 8.62 (s, 2H), 8.44 (s, 1H), 8.32 (t, 1H), 7.34-7.28 (m, 2H), 7.19-7.14 (m, 2H), 5.98-5.89 (m, 1H), 5.35 (d, 1H), 4.71-4.62 (m, 1H), 4.57-4.44 (m, 1H), 4.39-4.19 (m, 3H), 3.80-3.73 (m, 2H), 3.26-3.15 (m, 2H), 2.61 (d, 2H), 2.46-2.39 (m, 3H), 2.12- 1.99 (m, 2H), 1.41 (dd, 6H), 1.14 (d, 3H). | (S)-1-isopropyl-7- methyl-N-(4-(3- methylureido)benzyl)-5- (5-(2-oxopyrrolidin-1- yl)pyrimidin-2-yl)- 4,5,6,7-tetrahydro-1H- pyrazolo[4,3-c]pyridine- 3-carboxamide | 546.4 |
| 482 | Ex. 127 | 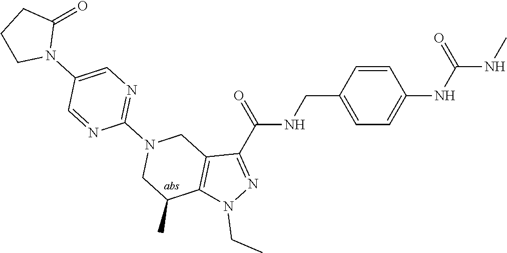 [SMILES: CCn1nc(C(=O)NCc2ccc(NC(=O)NC)cc2)c2c1[C@@H](C)CN(c1ncc(N3CCCC3=O)cn1)C2] ¹H NMR (400 MHz, (CD₃)₂SO) δ 8.62 (s, 2H), 8.45-8.39 (m, 2H), 7.33-7.28 (m, 2H), 7.19-7.12 (m, 2H), 5.94 (q, 1H), 5.29 (d, 1H), 4.59 (dd, 1H), 4.37-4.24 (m, 3H), 4.15-4.03 (m, 2H), 3.80-3.74 (m, 2H), 3.44-3.36 (m, 1H), 3.24-3.15 (m, 1H), 2.61 (d, 3H), 2.47-2.38 (m, 2H), 2.14- 2.02 (m, 2H), 1.37 (t, 3H), 1.15 (d, 3H). | (S)-1-ethyl-7-methyl-N- (4-(3- methylureido)benzyl)-5- (5-(2-oxopyrrolidin-1- yl)pyrimidin-2-yl)- 4,5,6,7-tetrahydro-1H- pyrazolo[4,3-c]pyridine- 3-carboxamide | 532.3 |
| 483 | Ex. 127 | 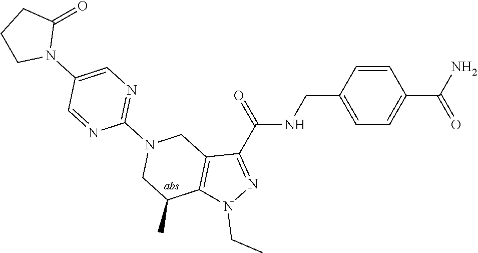 [SMILES: CCn1nc(C(=O)NCc2ccc(C(N)=O)cc2)c2c1[C@@H](C)CN(c1ncc(N3CCCC3=O)cn1)C2] ¹H NMR (400 MHz, (CD₃)₂SO) δ 8.67- 8.60 (m, 3H), 7.91 (s, 1H), 7.83-7.79 (m, 2H), 7.38-7.27 (m, 3H), 5.29 (d, 1H), 4.65- 4.54 (m, 1H), 4.50-4.37 (m, 2H), 4.30 (d, 1H), 4.17-4.03 (m, 2H), 3.76 (t, 2H), 3.45- 3.37 (m, 1H), 3.26-3.15 (m, 1H), 2.46- 2.39 (m, 2H), 2.12-2.00 (m, 2H), 1.38 (t, 3H), 1.15 (d, 3H). | (S)-N-(4- carbamoylbenzyl)-1- ethyl-7-methyl-5-(5-(2- oxopyrrolidin-1- yl)pyrimidin-2-yl)- 4,5,6,7-tetrahydro-1H- pyrazolo[4,3-c]pyridine- 3-carboxamide | 503.4 |
| 484 | Ex. 127 | 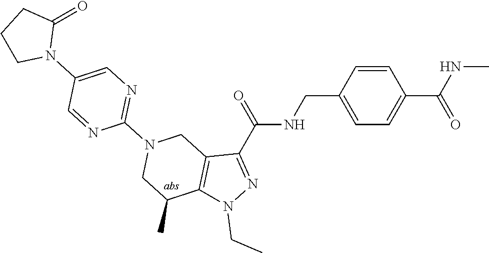 [SMILES: CCn1nc(C(=O)NCc2ccc(C(=O)NC)cc2)c2c1[C@@H](C)CN(c1ncc(N3CCCC3=O)cn1)C2] ¹H NMR (400 MHz, (CD₃)₂SO) δ 8.67- 8.58 (m, 3H), 8.36 (q, 1H), 7.79-7.73 (m, 2H), 7.39-7.32 (m, 2H), 5.29 (d, 1H), 4.63- 4.55 (m, 1H), 4.51-4.37 (m, 2H), 4.33- 4.24 (m, 1H), 4.16-4.01 (m, 2H), 3.76 (t, 2H), 3.45-3.35 (m, 1H), 3.27-3.16 (m, 1H), 2.76 (d, 3H), 2.46-2.37 (m, 2H), 2.13- 2.00 (m, 2H), 1.38 (t, 3H), 1.15 (d, 3H). | (S)-1-ethyl-7-methyl-N- (4- (methylcarbamoyl)benzyl)- 5-(5-(2-oxopyrrolidin- 1-yl)pyrimidin-2-yl)- 4,5,6,7-tetrahydro-1H- pyrazolo[4,3-c]pyridine- 3-carboxamide | 517.3 |
| 485 | Ex. 485 | 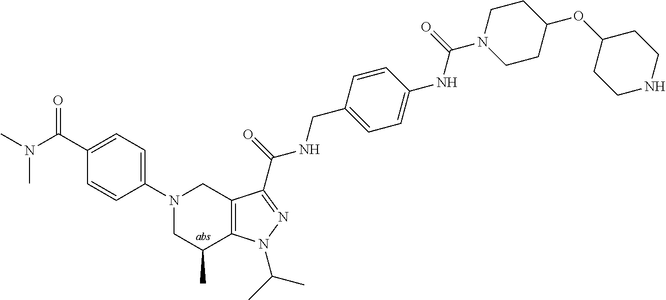 [SMILES: CC(C)n1nc(C(=O)NCc2ccc(NC(=O)N3CCC(OC4CCNCC4)CC3)cc2)c2c1[C@@H](C)CN(c1ccc(C(=O)N(C)C)cc1)C2] 1H NMR* (400 MHz, (CD₃)₂SO) δ 8.70- 8.50 (m, 2H), 8.42-8.34 (m, 1H), 8.24 (t, 1H), 7.31-7.19 (m, 4H), 7.12-7.05 (m, 2H), 6.89-6.83 (m, 2H), 4.50-4.38 (m, 2H), 4.31-4.19 (m, 2H), 4.00 (d, 1H), 3.76- 3.59 (m, 4H), 3.58-3.50 (m, 1H), 3.21- 2.93 (m, 6H), 2.92-2.80 (m, 8H), 1.86- 1.77 (m, 2H), 1.74-1.66 (m, 2H), 1.60- 1.48 (m, 2H), 1.37-1.25 (m, 7H), 1.18 (d, 3H). | (S)-5-(4- (dimethylcarbamoyl) phenyl)-1-isopropyl-7- methyl-N-(4-(4- (piperidin-4- yloxy)piperidine-1- carboxamido)benzyl)- 4,5,6,7-tetrahydro-1H- pyrazolo[4,3-c]pyridine- 3-carboxamide | 685.5 |
| 486 | Ex. 127 | 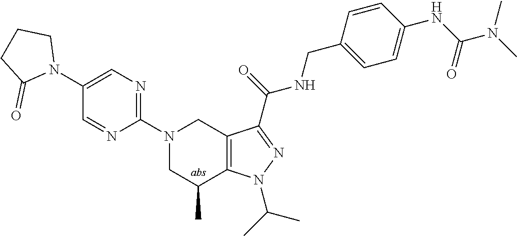 [SMILES: CC(C)n1nc(C(=O)NCc2ccc(NC(=O)N(C)C)cc2)c2c1[C@@H](C)CN(c1ncc(N3CCCC3=O)cn1)C2] ¹H NMR (400 MHz, (CD₃)₂SO) δ 8.62 (s, 2H), 8.33 (t, 1H), 8.23 (s, 1H), 7.41-7.35 (m, 2H), 7.19-7.13 (m, 2H), 5.35 (d, 1H), 4.71-4.63 (m, 1H), 4.57-4.46 (m, 1H), 4.41-4.20 (m, 3H), 3.77 (t, 2H), 3.41- 3.35 (m, 1H), 3.25-3.17 (m, 1H), 2.90 (s, 6H), 2.46-2.38 (m, 2H), 2.14-1.99 (m, 2H), 1.41 (dd, 6H), 1.14 (d, 3H). | (S)-N-(4-(3,3- dimethylureido)benzyl)- 1-isopropyl-7-methyl-5- (5-(2-oxopyrrolidin-1- yl)pyrimidin-2-yl)- 4,5,6,7-tetrahydro-1H- pyrazolo[4,3-c]pyridine- 3-carboxamide | 560.4 |
| 487 | Ex. 127 | 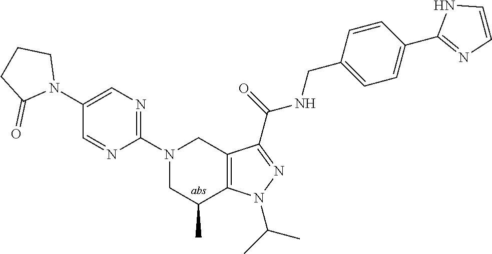 [SMILES: CC(C)n1nc(C(=O)NCc2ccc(-c3ncc[nH]3)cc2)c2c1[C@@H](C)CN(c1ncc(N3CCCC3=O)cn1)C2] ¹H NMR (400 MHz, (CD₃)₂SO) δ 12.44 (s, 1H), 8.62 (s, 2H), 8.49 (t, 1H), 7.89-7.83 (m, 2H), 7.40-7.32 (m, 2H), 7.21 (s, 1H), 6.98 (s, 1H), 5.35 (d, 1H), 4.72-4.64 (m, 1H), 4.57-4.36 (m, 3H), 4.25 (d, 1H), 3.76 (t, 2H), 3.39-3.34 (m, 1H), 3.26-3.14 (m, 1H), 2.46-2.37 (m, 2H), 2.15-2.00 (m, 2H), 1.46-1.34 (m, 6H), 1.14 (d, 3H). | (S)-N-(4-(1H-imidazol-2- yl)benzyl)-1-isopropyl-7- methyl-5-(5-(2- oxopyrrolidin-1- yl)pyrimidin-2-yl)- 4,5,6,7-tetrahydro-1H- pyrazolo[4,3-c]pyridine- 3-carboxamide | 540.5 |
| 488 | Ex. 127 | 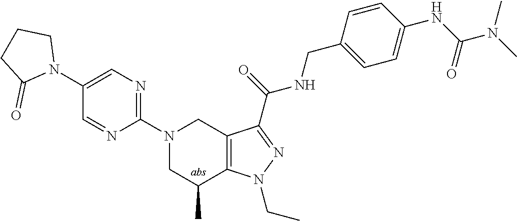 [SMILES: CCn1nc(C(=O)NCc2ccc(NC(=O)N(C)C)cc2)c2c1[C@@H](C)CN(c1ncc(N3CCCC3=O)cn1)C2] ¹H NMR (400 MHz, (CD₃)₂SO) δ 8.63 (s, 2H), 8.43 (t, 1H), 8.22 (s, 1H), 7.41-7.35 (m, 2H), 7.20-7.13 (m, 2H), 5.30 (d, 1H), 4.60 (dd, 1H), 4.39-4.25 (m, 3H), 4.14- 4.02 (m, 2H), 3.80-3.74 (m, 2H), 3.45- 3.36 (m, 1H), 3.25-3.14 (m, 1H), 2.90 (s, 6H), 2.46-2.39 (m, 2H), 2.13-2.00 (m, 2H), 1.38 (t, 3H), 1.15 (d, 3H). | (S)-N-(4-(3,3- dimethylureido)benzyl)- 1-ethyl-7-methyl-5-(5-(2- oxopyrrolidin-1- yl)pyrimidin-2-yl)- 4,5,6,7-tetrahydro-1H- pyrazolo[4,3-c]pyridine- 3-carboxamide | 546.3 |
| 489 | Ex. 127 | 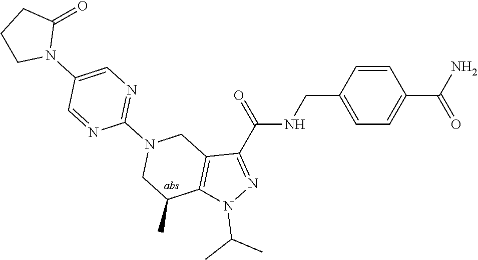 [SMILES: CC(C)n1nc(C(=O)NCc2ccc(C(N)=O)cc2)c2c1[C@@H](C)CN(c1ncc(N3CCCC3=O)cn1)C2] ¹H NMR (400 MHz, (CD₃)₂SO) δ 8.62 (s, 2H), 8.53 (t, 1H), 7.91 (s, 1H), 7.84-7.79 (m, 2H), 7.41-7.33 (m, 2H), 7.30 (s, 1H), 5.34 (d, 1H), 4.72-4.63 (m, 1H), 4.59- 4.38 (m, 3H), 4.24 (d, 1H), 3.77 (t, 2H), 3.27- 3.16 (m, 2H), 2.46-2.38 (m, 2H), 2.13- 2.00 (m, 2H), 1.42 (dd, 6H), 1.14 (d, 3H). | (S)-N-(4- carbamoylbenzyl)-1- isopropyl-7-methyl-5-(5- (2-oxopyrrolidin-1- yl)pyrimidin-2-yl)- 4,5,6,7-tetrahydro-1H- pyrazolo[4,3-c]pyridine- 3-carboxamide | 517.3 |
| 490 | Ex. 127 | 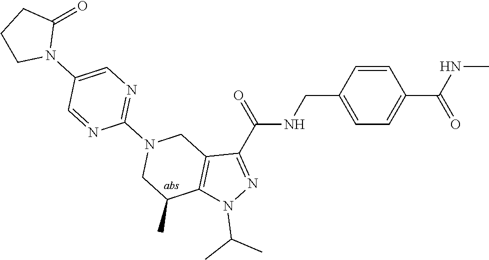 [SMILES: CNC(=O)c1ccc(CNC(=O)c2nn(C(C)C)c3c2CN(c2ncc(N4CCCC4=O)cn2)C[C@@H]3C)cc1] ¹H NMR (400 MHz, (CD₃)₂SO) δ 8.62 (s, 2H), 8.53 (t, 1H), 8.40-8.34 (m, 1H), 7.81- 7.73 (m, 2H), 7.40-7.33 (m, 2H), 5.34 (d, 1H), 4.71-4.64 (m, 1H), 4.58-4.38 (m, 3H), 4.24 (d, 1H), 3.81-3.73 (m, 2H), 3.27- 3.16 (m, 2H), 2.76 (d, 3H), 2.47-2.39 (m, 2H), 2.12-2.00 (m, 2H), 1.47-1.34 (m, 6H), 1.14 (d, 3H). | (S)-1-isopropyl-7- methyl-N-(4- (methylcarbamoyl)benzyl)- 5-(5-(2-oxopyrrolidin- 1-yl)pyrimidin-2-yl)- 4,5,6,7-tetrahydro-1H- pyrazolo[4,3-c]pyridine- 3-carboxamide | 531.3 |
| 491 | Ex. 126 | 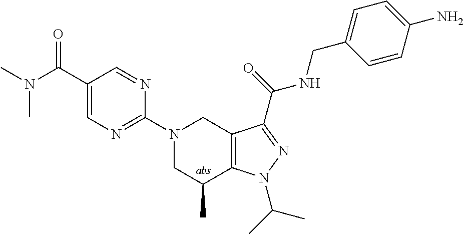 [SMILES: CC(C)n1nc(C(=O)NCc2ccc(N)cc2)c2c1[C@@H](C)CN(c1ncc(C(=O)N(C)C)cn1)C2] ¹H NMR (400 MHz, (CD₃)₂SO) δ 8.47 (s, 2H), 8.10 (t, 1H), 6.98-6.91 (m, 2H), 6.49- 6.41 (m, 2H), 5.42 (d, 1H), 4.90 (s, 2H), 4.74-4.65 (m, 1H), 4.52-4.41 (m, 1H), 4.31-4.12 (m, 3H), 3.23-3.17 (m, 1H), 3.01-2.88 (m, 7H), 1.39-1.30 (m, 6H), 1.12-1.07 (m, 3H). | (S)-N-(4-aminobenzyl)- 5-(5- (dimethylcarbamoyl) pyrimidin-2-yl)-1-isopropyl- 7-methyl-4,5,6,7- tetrahydro-1H- pyrazolo[4,3-c]pyridine- 3-carboxamide | 477.2 |
| 492 | Ex. 127 | 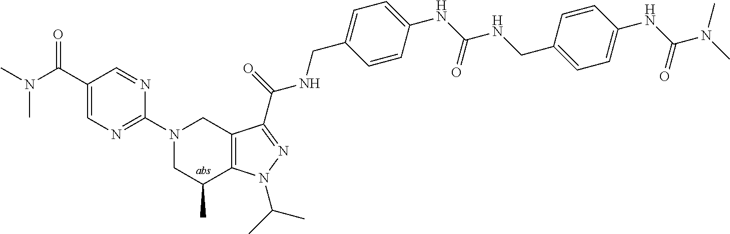 ¹H NMR (400 MHz, (CD₃)₂SO) δ 8.48 (s, 2H), 8.43 (s, 1H), 8.31 (t, 1H), 8.20 (s, 1H), 7.39-7.32 (m, 2H), 7.32-7.26 (m, 2H), 7.19-7.07 (m, 4H), 6.43 (t, 1H), 5.43 (d, 1H), 4.75-4.68 (m, 1H), 4.54-4.43 (m, 1H), 4.37-4.23 (m, 3H), 4.16 (d, 2H), 3.29- 3.21 (m, 1H), 2.96 (s, 6H), 2.89-2.82 (m, 7H), 1.38 (dd, 6H), 1.11 (d, 3H). | (S)-5-(5- (dimethylcarbamoyl) pyrimidin-2-yl)-N-(4-(3-(4- (3,3- dimethylureido)benzyl) ureido)benzyl)-1- isopropyl-7-methyl- 4,5,6,7-tetrahydro-1H- pyrazolo[4,3-c]pyridine- 3-carboxamide | 696.5 |
| 493^{∧} | Ex. 39^{l} | 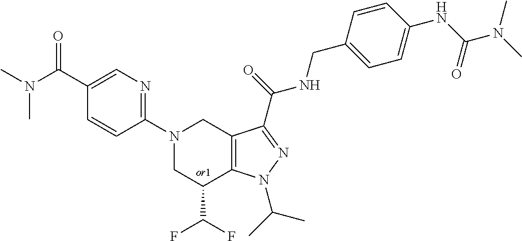 ¹H NMR (400 MHz, (CD₃)₂SO) δ 8.47 (t, 1H), 8.27 (d, 1H), 8.22 (s, 1H), 7.68 (dd, 1H), 7.42-7.35 (m, 2H), 7.20-7.14 (m, 2H), 6.83 (d, 1H), 6.25-5.89 (m, 1H), 5.10- 4.98 (m, 2H), 4.71-4.56 (m, 1H), 4.42- 4.16 (m, 3H), 3.91-3.79 (m, 1H), 3.23- 3.17 (m, 1H), 2.98 (s, 6H), 2.90 (s, 6H), 1.46-1.35 (m, 6H). | rel-(R or S)-7- (difluoromethyl)-5-(5- (dimethylcarbamoyl) pyridin-2-yl)-N-(4-(3,3- dimethylureido)benzyl)- 1-isopropyl-4,5,6,7- tetrahydro-1H- pyrazolo[4,3-c]pyridine- 3-carboxamide | 583.3 |
| 494^{∧} | Ex. 39^{l} | 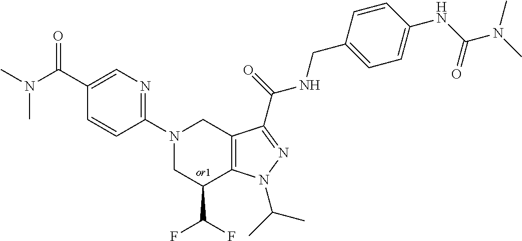 ¹H NMR (400 MHz, (CD₃)₂SO) δ 8.47 (t, 1H), 8.26 (d, 1H), 8.22 (s, 1H), 7.68 (dd, 1H), 7.41-7.34 (m, 2H), 7.20-7.14 (m, 2H), 6.82 (d, 1H), 6.27-5.83 (m, 1H), 5.09- 4.96 (m, 2H), 4.67-4.57 (m, 1H), 4.41- 4.19 (m, 3H), 3.92-3.79 (m, 1H), 3.23- 3.15 (m, 1H), 2.97 (s, 6H), 2.89 (s, 6H), 1.48-1.34 (m, 6H). | rel-(R or S)-7- (difluoromethyl)-5-(5- (dimethylcarbamoyl) pyridin-2-yl)-N-(4-(3,3- dimethylureido)benzyl)- 1-isopropyl-4,5,6,7- tetrahydro-1H- pyrazolo[4,3-c]pyridine- 3-carboxamide | 583.3 |
| 495 | Ex. 127 | 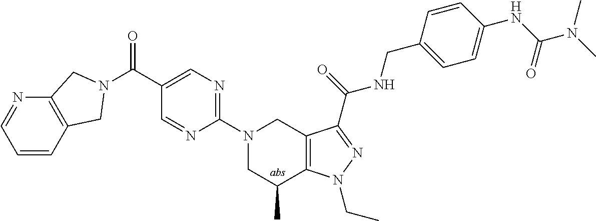 [SMILES: CCn1nc(C(=O)NCc2ccc(NC(=O)N(C)C)cc2)c2c1[C@@H](C)CN(c1ncc(C(=O)N3Cc4cccnc4C3)cn1)C2] ¹H NMR (400 MHz, (CD₃)₂SO) δ 8.74 (d, 2H), 8.49-8.41 (m, 2H), 8.22 (s, 1H), 7.86- 7.68 (m, 1H), 7.40-7.35 (m, 2H), 7.34- 7.27 (m, 1H), 7.19-7.13 (m, 2H), 5.45 (d, 1H), 5.05-4.66 (m, 5H), 4.46-4.24 (m, 3H), 4.16-4.04 (m, 2H), 3.55-3.46 (m, 1H), 2.91-2.85 (m, 7H), 1.38 (t, 3H), 1.17 (d, 3H). | (S)-5-(5-(6,7-dihydro- 5H-pyrrolo[3,4- b]pyridine-6- carbonyl)pyrimidin-2-yl)- N-(4-(3,3- dimethylureido)benzyl)- 1-ethyl-7-methyl-4,5,6,7- tetrahydro-1H- pyrazolo[4,3-c]pyridine- 3-carboxamide | 609.4 |
| 496 | Ex. 127 | 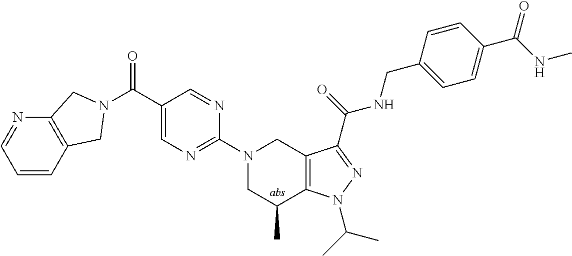 [SMILES: CNC(=O)c1ccc(CNC(=O)c2nn(C(C)C)c3c2CN(c2ncc(C(=O)N4Cc5cccnc5C4)cn2)C[C@@H]3C)cc1] ¹H NMR (400 MHz, (CD₃)₂SO) δ 8.76- 8.69 (m, 2H), 8.58-8.52 (m, 1H), 8.48- 8.43 (m, 1H), 8.39-8.34 (m, 1H), 7.86- 7.68 (m, 3H), 7.40-7.27 (m, 3H), 5.49 (d, 1H), 5.06-4.73 (m, 5H), 4.58-4.27 (m, 4H), 3.50-3.39 (m, 2H), 2.76 (d, 3H), 1.43 (dd, 6H), 1.16 (d, 3H). | (S)-5-(5-(6,7-dihydro- 5H-pyrrolo[3,4- b]pyridine-6- carbonyl)pyrimidin-2-yl)- 1-isopropyl-7-methyl-N- (4- (methylcarbamoyl)benzyl)- 4,5,6,7-tetrahydro-1H- pyrazolo[4,3-c]pyridine- 3-carboxamide | 594.3 |
| 497 | Ex. 236 | 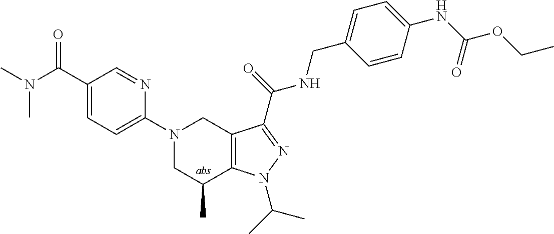 [SMILES: CCOC(=O)Nc1ccc(CNC(=O)c2nn(C(C)C)c3c2CN(c2ccc(C(=O)N(C)C)cn2)C[C@@H]3C)cc1] ¹H NMR (400 MHz, (CD₃)₂SO) δ 9.54 (s, 1H), 8.36 (t, 1H), 8.23 (d, 1H), 7.62 (dd, 1H), 7.37 (d, 2H), 7.21 (d, 2H), 6.87 (d, 1H), 5.07 (d, 1H), 4.57-4.47 (m, 1H), 4.45- 4.27 (m, 3H), 4.25-4.17 (m, 1H), 4.09 (q, 2H), 3.26-3.14 (m, 2H), 2.96 (s, 6H), 1.40 (dd, 6H), 1.24-1.16 (m, 6H). | ethyl (S)-(4-((5-(5- (dimethylcarbamoyl) pyridin-2-yl)-1-isopropyl-7- methyl-4,5,6,7- tetrahydro-1H- pyrazolo[4,3-c]pyridine- 3- carboxamido)methyl) phenyl)carbamate | 548.4 |
| 498 | Ex. 498 | 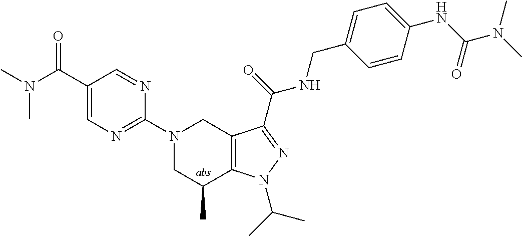 ¹H NMR (400 MHz, (CD₃)₂SO) δ 8.80 (s, 2H), 8.36 (t, 1H), 8.24 (s, 1H), 7.42-7.36 (m, 2H), 7.20-7.14 (m, 2H), 5.54 (d, 1H), 4.81 (d, 1H), 4.57-4.46 (m, 1H), 4.40- 4.27 (m, 3H), 3.49-3.41 (m, 1H), 3.32- 3.22 (m, 1H), 2.94-2.86 (m, 6H), 1.41 (dd, 6H), 1.13 (d, 3H). | (S)-2-(3-((4-(3,3- dimethylureido)benzyl) carbamoyl)-1-isopropyl-7- methyl-1,4,6,7- tetrahydro-5H- pyrazolo[4,3-c]pyridin-5- yl)pyrimidine-5- carboxylic acid | 521.3 |
| 499 | Ex. 127 | 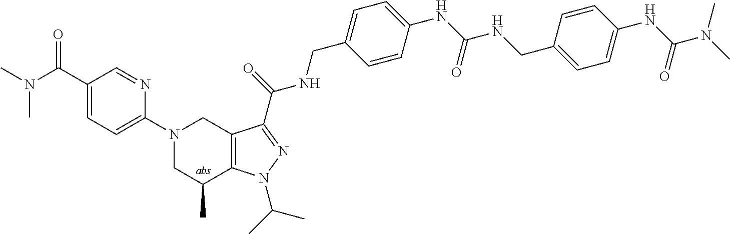 ¹H NMR (400 MHz, (CD₃)₂SO) δ 8.48 (s, 1H), 8.34 (t, 1H), 8.26-8.21 (m, 2H), 7.64 (dd, 1H), 7.42-7.37 (m, 2H), 7.36-7.31 (m, 2H), 7.21-7.11 (m, 4H), 6.89 (d, 1H), 6.48 (t, 1H), 5.09 (d, 1H), 4.58-4.48 (m, 1H), 4.46-4.16 (m, 7H), 3.27-3.17 (m, 1H), 3.03-2.94 (m, 6H), 2.93-2.84 (m, 6H), 1.42 (dd, 6H), 1.20 (d, 3H). | (S)-5-(5- (dimethylcarbamoyl) pyridin-2-yl)-N-(4-(3-(4-(3,3- dimethylureido)benzyl) ureido)benzyl)-1- isopropyl-7-methyl- 4,5,6,7-tetrahydro-1H- pyrazolo[4,3-c]pyridine- 3-carboxamide | 695.4 |
| 500 | Ex. 395 | 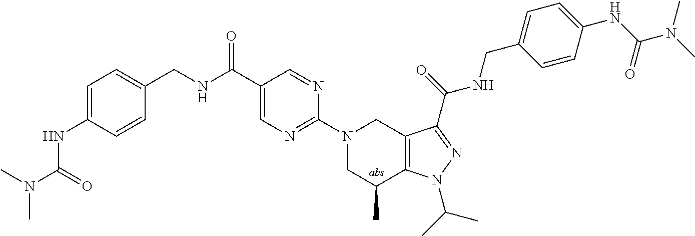 ¹H NMR (400 MHz, (CD₃)₂SO) δ 8.88- 8.79 (m, 3H), 8.35 (t, 1H), 8.23-8.20 (m, 2H), 7.41-7.35 (m, 4H), 7.19-7.12 (m, 4H), 5.49 (d, 1H), 4.79 (d, 1H), 4.55-4.45 (m, 1H), 4.42-4.27 (m, 5H), 3.45-3.37 (m, 1H), 3.27-3.20 (m, 1H), 2.93-2.83 (m, 12H), 1.41 (dd, 6H), 1.12 (d, 3H). | (S)-N-(4-(3,3- dimethylureido)benzyl)- 5-(5-((4-(3,3- dimethylureido)benzyl) carbamoyl)pyrimidin-2- yl)-1-isopropyl-7-methyl- 4,5,6,7-tetrahydro-1H- pyrazolo[4,3-c]pyridine- 3-carboxamide | 696.5 |
| 501 | Ex. 127 | 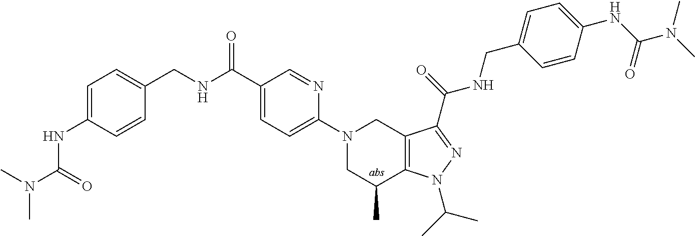 ¹H NMR (400 MHz, (CD₃)₂SO) δ 8.75 (t, 1H), 8.65 (d, 1H), 8.36 (t, 1H), 8.25-8.19 (m, 2H), 8.01 (dd, 1H), 7.41-7.34 (m, 4H), 7.21-7.11 (m, 4H), 6.91 (d, 1H), 5.13 (d, 1H), 4.59-4.44 (m, 2H), 4.41-4.22 (m, 5H), 3.26-3.19 (m, 2H), 2.92-2.84 (m, 12H), 1.42 (dd, 6H), 1.18 (d, 3H). | (S)-N-(4-(3,3- dimethylureido)benzyl)- 5-(5-((4-(3,3- dimethylureido)benzyl) carbamoyl)pyridin-2-yl)-1- isopropyl-7-methyl- 4,5,6,7-tetrahydro-1H- pyrazolo[4,3-c]pyridine- 3-carboxamide | 695.4 |
| 502 | Ex. 127 | 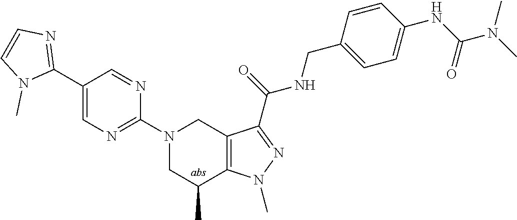 [SMILES: C[C@H]1CN(c2ncc(-c3nccn3C)cn2)Cc2c(C(=O)NCc3ccc(NC(=O)N(C)C)cc3)nn(C)c21] ¹H NMR (400 MHz, (CD₃)₂SO) δ 8.52 (s, 2H), 8.49-8.44 (m, 1H), 8.22-8.19 (m, 1H), 7.85-7.81 (m, 1H), 7.39-7.33 (m, 2H), 7.16-7.07 (m, 3H), 5.39-5.30 (m, 1H), 4.64-4.57 (m, 1H), 4.43-4.27 (m, 3H), 3.80 (s, 3H), 3.64 (s, 3H), 3.54-3.44 (m, 2H), 2.89 (s, 6H), 1.16 (d, 3H). | (S)-N-(4-(3,3- dimethylureido)benzyl)- 1,7-dimethyl-5-(5-(1- methyl-1H-imidazol-2- yl)pyrimidin-2-yl)- 4,5,6,7-tetrahydro-1H- pyrazolo[4,3-c]pyridine- 3-carboxamide | 529.3 |
| 503 | Ex. 236 | 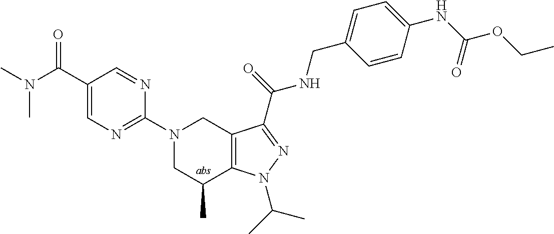 [SMILES: CCOC(=O)Nc1ccc(CNC(=O)c2nn(C(C)C)c3c2CN(c2ncc(C(=O)N(C)C)cn2)C[C@@H]3C)cc1] ¹H NMR (400 MHz, (CD₃)₂SO) δ 9.54 (s, 1H), 8.51 (s, 2H), 8.37 (t, 1H), 7.38 (d, 2H), 7.24-7.20 (m, 2H), 5.46 (d, 1H), 4.78- 4.69 (m, 1H), 4.57-4.46 (m, 1H), 4.41- 4.27 (m, 3H), 4.10 (q, 2H), 3.47-3.36 (m, 1H), 3.26-3.23 (m, 1H), 2.99 (s, 6H), 1.41 (dd, 6H), 1.22 (t, 3H), 1.14 (d, 3H). | ethyl (S)-(4-((5-(5- (dimethylcarbamoyl) pyrimidin-2-yl)-1-isopropyl- 7-methyl-4,5,6,7- tetrahydro-1H- pyrazolo[4,3-c]pyridine- 3- carboxamido)methyl) phenyl)carbamate | 549.3 |
| 504 | Ex. 498 | 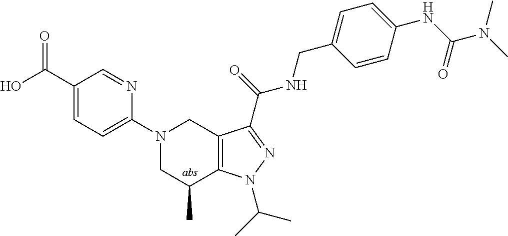 [SMILES: CC(C)n1nc(C(=O)NCc2ccc(NC(=O)N(C)C)cc2)c2c1[C@@H](C)CN(c1ccc(C(=O)O)cn1)C2] ¹H NMR (400 MHz, (CD₃)₂SO) δ 8.58- 8.54 (m, 1H), 8.41 (t, 1H), 8.25 (s, 1H), 8.08- 8.01 (m, 1H), 7.42-7.37 (m, 2H), 7.20- 7.15 (m, 2H), 7.14-7.09 (m, 1H), 5.15 (d, 1H), 4.60-4.46 (m, 2H), 4.40-4.27 (m, 3H), 3.51-3.43 (m, 1H), 3.34-3.25 (m, 1H), 2.90 (s, 6H), 1.45-1.39 (m, 6H), 1.20 (d, 3H). | (S)-6-(3-((4-(3,3- dimethylureido)benzyl) carbamoyl)-1-isopropyl-7- methyl-1,4,6,7- tetrahydro-5H- pyrazolo[4,3-c]pyridin-5- yl)nicotinic acid | 520.3 |
| 505 | Ex. 469ⁿ | 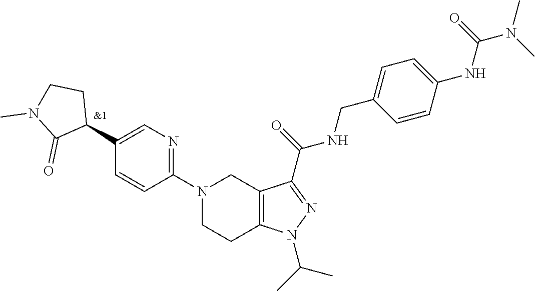 ¹H NMR (400 MHz, CDCl₃) δ 8.08 (d, 1H), 7.43-7.39 (m, 1H), 7.38-7.33 (m, 2H), 7.32-7.28 (m, 2H), 7.18-7.11 (m, 1H), 6.78 (d, 1H), 6.29 (s, 1H), 4.69 (s, 2H), 4.56 (d, 2H), 4.42-4.31 (m, 1H), 4.09 (t, 2H), 3.63-3.48 (m, 2H), 3.37 (s, 3H), 3.17- 3.07 (m, 1H), 3.03 (s, 6H), 2.83-2.75 (m, 3H), 2.68-2.59 (m, 1H), 1.44 (d, 6H). | rac-(R)-N-(4-(3,3- dimethylureido)benzyl)- 1-isopropyl-5-(5-(1- methyl-2-oxopyrrolidin- 3-yl)pyridin-2-yl)-4,5,6,7- tetrahydro-1H- pyrazolo[4,3-c]pyridine- 3-carboxamide | 559.3 |
| 506^{∧} | Ex. 113^{f} | 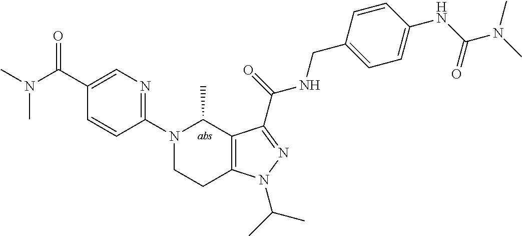 ¹H NMR (400 MHz, (CD₃)₂SO) δ 8.31 (t, 1H), 8.23 (s, 2H), 7.64-7.58 (m, 1H), 7.42- 7.36 (m, 2H), 7.20-7.15 (m, 2H), 6.87- 6.82 (m, 1H), 5.83-5.72 (m, 1H), 4.67- 4.56 (m, 1H), 4.52-4.24 (m, 3H), 3.40- 3.34 (m, 1H), 2.97 (s, 6H), 2.91 (d, 6H), 2.85-2.69 (m, 2H), 1.43 (d, 6H), 1.35 (d, 3H). | (R)-5-(5- (dimethylcarbamoyl) pyridin-2-yl)-N-(4-(3,3- dimethylureido)benzyl)- 1-isopropyl-4-methyl- 4,5,6,7-tetrahydro-1H- pyrazolo[4,3-c]pyridine- 3-carboxamide | 547.4 |
| 507^{∧} | Ex. 113^{f} | 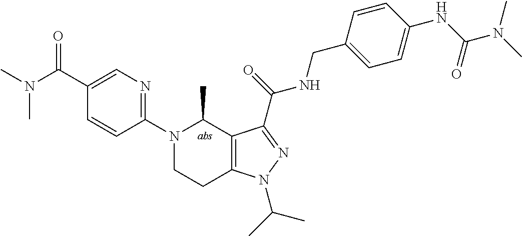 ¹H NMR (400 MHz, (CD₃)₂SO) δ 8.31 (t, 1H), 8.24-8.21 (m, 2H), 7.62 (dd, 1H), 7.42-7.36 (m, 2H), 7.20-7.15 (m, 2H), 6.85 (d, 1H), 5.84-5.72 (m, 1H), 4.67- 4.55 (m, 1H), 4.52-4.26 (m, 3H), 3.39- 3.34 (m, 1H), 2.97 (s, 6H), 2.91 (s, 6H), 2.86-2.64 (m, 2H), 1.43 (d, 6H), 1.35 (d, 3H). | (S)-5-(5- (dimethylcarbamoyl) pyridin-2-yl)-N-(4-(3,3- dimethylureido)benzyl)- 1-isopropyl-4-methyl- 4,5,6,7-tetrahydro-1H- pyrazolo[4,3-c]pyridine- 3-carboxamide | 547.4 |
| 508^{∧} | Ex. 113 | 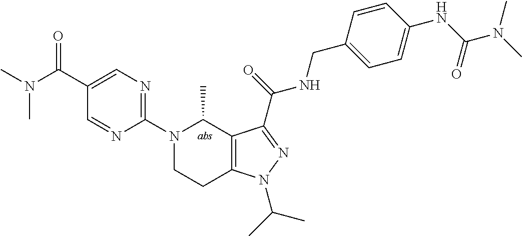 1H NMR (400 MHz, (CD₃)₂SO) δ 8.51 (s, 2H), 8.32 (t, 1H), 8.23 (s, 1H), 7.42-7.36 (m, 2H), 7.19-7.14 (m, 2H), 6.09 (q, 1H), 5.00 (dd, 1H), 4.53-4.26 (m, 3H), 3.30- 3.23 (m, 1H), 2.99 (s, 6H), 2.93-2.87 (m, 6H), 2.86-2.70 (m, 2H), 1.46-1.40 (m, 6H), 1.35 (d, 3H). | (R)-5-(5- (dimethylcarbamoyl) pyrimidin-2-yl)-N-(4-(3,3- dimethylureido)benzyl)- 1-isopropyl-4-methyl- 4,5,6,7-tetrahydro-1H- pyrazolo[4,3-c]pyridine- 3-carboxamide | 548.4 |
| 509^{∧} | Ex. 113 | 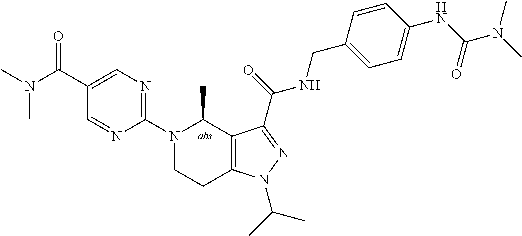 ¹H NMR (400 MHz, (CD₃)₂SO) δ 8.51 (s, 2H), 8.32 (t, 1H), 8.23 (s, 1H), 7.42-7.37 (m, 2H), 7.19-7.14 (m, 2H), 6.09 (q, 1H), 5.00 (dd, 1H), 4.53-4.25 (m, 3H), 3.31- 3.26 (m, 1H), 2.99 (s, 6H), 2.92-2.89 (m, 6H), 2.89-2.69 (m, 2H), 1.46-1.41 (m, 6H), 1.35 (d, 3H). | (S)-5-(5- (dimethylcarbamoyl) pyrimidin-2-yl)-N-(4-(3,3- dimethylureido)benzyl)- 1-isopropyl-4-methyl- 4,5,6,7-tetrahydro-1H- pyrazolo[4,3-c]pyridine- 3-carboxamide | 548.4 |
| 510^{∧} | Ex. 113 | 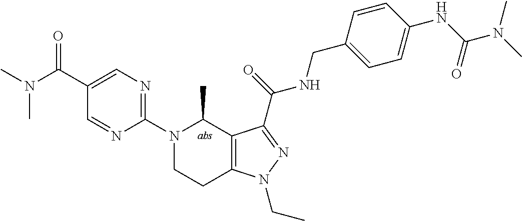 ¹H NMR (400 MHz, (CD₃)₂SO) δ 8.51 (s, 2H), 8.40 (t, 1H), 8.22 (s, 1H), 7.41-7.36 (m, 2H), 7.19-7.14 (m, 2H), 6.13-6.07 (m, 1H), 5.05-4.95 (m, 1H), 4.43-4.24 (m, 3H), 4.12-3.99 (m, 2H), 2.99 (s, 6H), 2.91 (s, 6H), 2.87-2.70 (m, 2H), 1.44 (d, 3H), 1.32 (t, 3H). | (S)-5-(5- (dimethylcarbamoyl) pyrimidin-2-yl)-N-(4-(3,3- dimethylureido)benzyl)- 1-ethyl-4-methyl-4,5,6,7- tetrahydro-1H- pyrazolo[4,3-c]pyridine- 3-carboxamide | 534.3 |
| 511^{∧} | Ex. 113 | 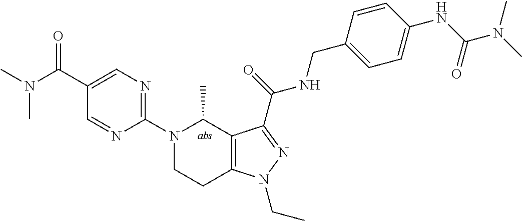 ¹H NMR (400 MHz, (CD₃)₂SO) δ 8.52 (s, 2H), 8.41 (t, 1H), 8.26-8.20 (m, 1H), 7.42- 7.37 (m, 2H), 7.20-7.14 (m, 2H), 6.10 (q, 1H), 5.06-4.97 (m, 1H), 4.44-4.25 (m, 3H), 4.12-4.00 (m, 2H), 2.99 (s, 6H), 2.91 (s, 6H), 2.88-2.71 (m, 2H), 1.44 (d, 3H), 1.33 (t, 3H). | (R)-5-(5- (dimethylcarbamoyl) pyrimidin-2-yl)-N-(4-(3,3- dimethylureido)benzyl)- 1-ethyl-4-methyl-4,5,6,7- tetrahydro-1H- pyrazolo[4,3-c]pyridine- 3-carboxamide | 534.3 |
| 512^{∧} | Ex. 537 | 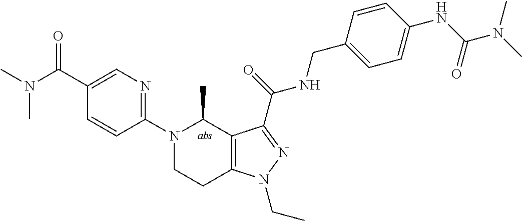 ¹H NMR (400 MHz, (CD₃)₂SO) δ 8.42- 8.36 (m, 1H), 8.24-8.20 (m, 2H), 7.64- 7.59 (m, 1H), 7.41-7.36 (m, 2H), 7.19- 7.13 (m, 2H), 6.87-6.83 (m, 1H), 5.86- 5.74 (m, 1H), 4.65-4.56 (m, 1H), 4.42- 4.23 (m, 3H), 4.10-4.01 (m, 2H), 2.97 (s, 6H), 2.91 (s, 6H), 2.79-2.71 (m, 2H), 1.43 (d, 3H), 1.33 (t, 3H). | (S)-5-(5- (dimethylcarbamoyl) pyridin-2-yl)-N-(4-(3,3- dimethylureido)benzyl)- 1-ethyl-4-methyl-4,5,6,7- tetrahydro-1H- pyrazolo[4,3-c]pyridine- 3-carboxamide | 533.4 |
| 513^{∧} | Ex. 537 | 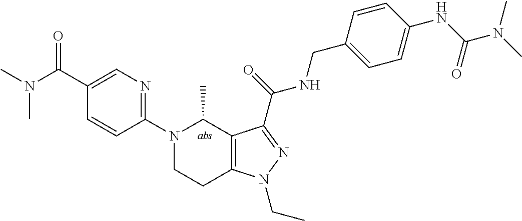 ¹H NMR (400 MHz, (CD₃)₂SO) δ 8.39 (t, 1H), 8.24-8.20 (m, 2H), 7.62 (dd, 1H), 7.42-7.35 (m, 2H), 7.16 (d, 2H), 6.85 (d, 1H), 5.85-5.76 (m, 1H), 4.64-4.56 (m, 1H), 4.43-4.25 (m, 3H), 4.10-4.01 (m, 2H), 2.97 (s, 6H), 2.91 (s, 6H), 2.81-2.72 (m, 2H), 1.43 (d, 3H), 1.32 (t, 3H). | (R)-5-(5- (dimethylcarbamoyl) pyridin-2-yl)-N-(4-(3,3- dimethylureido)benzyl)- 1-ethyl-4-methyl-4,5,6,7- tetrahydro-1H- pyrazolo[4,3-c]pyridine- 3-carboxamide | 533.3 |
| 514 | Ex. 18^{f} | 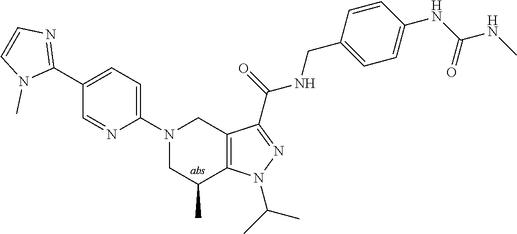 [SMILES: CNC(=O)Nc1ccc(CNC(=O)c2nn(C(C)C)c3c2CN(c2ccc(-c4nccn4C)cn2)C[C@@H]3C)cc1] | (S)-1-isopropyl-7- methyl-5-(5-(1-methyl- 1H-imidazol-2-yl)pyridin- 2-yl)-N-(4-(3- methylureido)benzyl)- 4,5,6,7-tetrahydro-1H- pyrazolo[4,3-c]pyridine- 3-carboxamide | 542.3 |
| 515 | Ex. 515 | 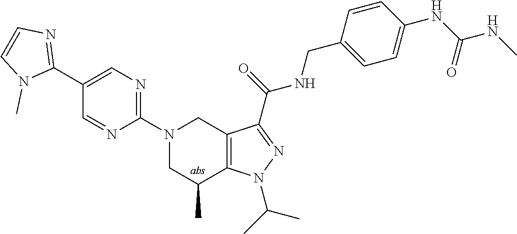 [SMILES: CNC(=O)Nc1ccc(CNC(=O)c2nn(C(C)C)c3c2CN(c2ncc(-c4nccn4C)cn2)C[C@@H]3C)cc1] ¹H NMR (600 MHz, (CD₃)₂SO) δ 8.77 (s, 1H), 8.44 (s, 1H), 8.38 (t, 1H), 7.81-7.77 (m, 2H), 7.34-7.30 (m, 2H), 7.19-7.15 (m, 2H), 5.97-5.93 (m, 1H), 5.55 (d, 1H), 4.82 (d, 1H), 4.57-4.50 (m, 1H), 4.41- 4.28 (m, 3H), 3.87-3.85 (m, 3H), 3.50- 3.47 (m, 2H), 3.34-3.28 (m, 1H), 2.62 (d, 3H), 1.43 (dd, 6H), 1.16 (d, 3H). | (S)-1-isopropyl-7- methyl-5-(5-(1-methyl- 1H-imidazol-2- yl)pyrimidin-2-yl)-N-(4- (3-methylureido)benzyl)- 4,5,6,7-tetrahydro-1H- pyrazolo[4,3-c]pyridine- 3-carboxamide | 543.5 |
| 516 | Ex. 18^{f} | 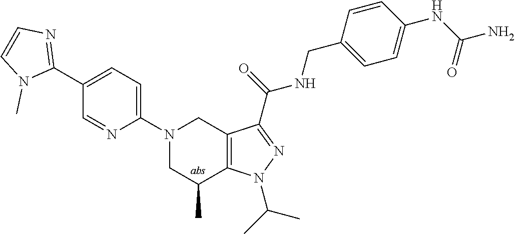 [SMILES: CC(C)n1nc(C(=O)NCc2ccc(NC(N)=O)cc2)c2c1[C@@H](C)CN(c1ccc(-c3nccn3C)cn1)C2] | (S)-1-isopropyl-7- methyl-5-(5-(1-methyl- 1H-imidazol-2-yl)pyridin- 2-yl)-N-(4-ureidobenzyl)- 4,5,6,7-tetrahydro-1H- pyrazolo[4,3-c]pyridine- 3-carboxamide | 528.4 |
| 517 | Ex. 77 | 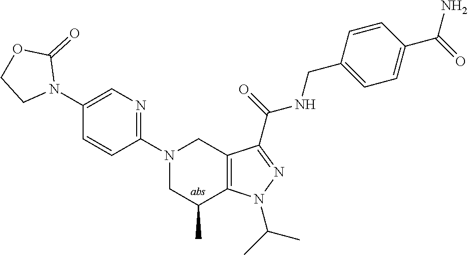 [SMILES: CC(C)n1nc(C(=O)NCc2ccc(C(N)=O)cc2)c2c1[C@@H](C)CN(c1ccc(N3CCOC3=O)cn1)C2] | (S)-N-(4- carbamoylbenzyl)-1- isopropyl-7-methyl-5-(5- (2-oxooxazolidin-3- yl)pyridin-2-yl)-4,5,6,7- tetrahydro-1H- pyrazolo[4,3-c]pyridine- 3-carboxamide | 518.4 |
| 518 | Ex. 77 | 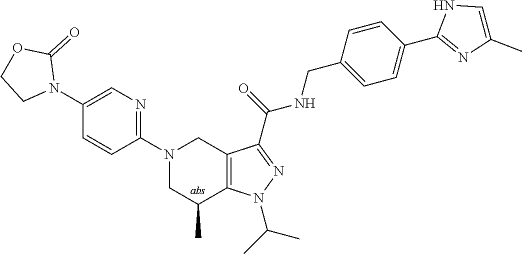 [SMILES: Cc1c[nH]c(-c2ccc(CNC(=O)c3nn(C(C)C)c4c3CN(c3ccc(N5CCOC5=O)cn3)C[C@@H]4C)cc2)n1] | (S)-1-isopropyl-7- methyl-N-(4-(4-methyl- 1H-imidazol-2- yl)benzyl)-5-(5-(2- oxooxazolidin-3- yl)pyridin-2-yl)-4,5,6,7- tetrahydro-1H- pyrazolo[4,3-c]pyridine- 3-carboxamide | 555.3 |
| 519 | Ex. 127 | 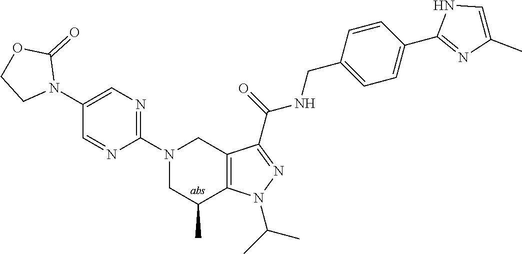 [SMILES: Cc1c[nH]c(-c2ccc(CNC(=O)c3nn(C(C)C)c4c3CN(c3ncc(N5CCOC5=O)cn3)C[C@@H]4C)cc2)n1] | (S)-1-isopropyl-7- methyl-N-(4-(4-methyl- 1H-imidazol-2- yl)benzyl)-5-(5-(2- oxooxazolidin-3- yl)pyrimidin-2-yl)- 4,5,6,7-tetrahydro-1H- pyrazolo[4,3-c]pyridine- 3-carboxamide | 556.3 |
| 520 | Ex. 127 | 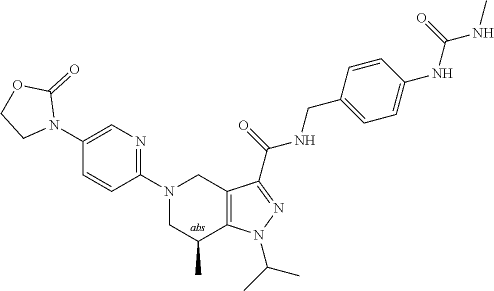 [SMILES: CNC(=O)Nc1ccc(CNC(=O)c2nn(C(C)C)c3c2CN(c2ccc(N4CCOC4=O)cn2)C[C@@H]3C)cc1] | (S)-1-isopropyl-7- methyl-N-(4-(3- methylureido)benzyl)-5- (5-(2-oxooxazolidin-3- yl)pyridin-2-yl)-4,5,6,7- tetrahydro-1H- pyrazolo[4,3-c]pyridine- 3-carboxamide | 547.5 |
| 521 | Ex. 127 | 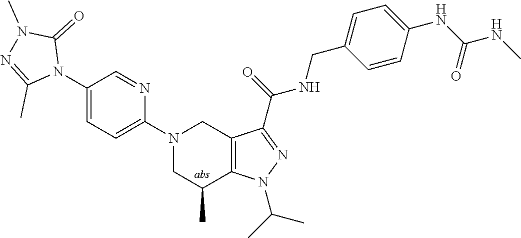 [SMILES: CNC(=O)Nc1ccc(CNC(=O)c2nn(C(C)C)c3c2CN(c2ccc(-n4c(C)nn(C)c4=O)cn2)C[C@@H]3C)cc1] | (S)-5-(5-(1,3-dimethyl-5- oxo-1,5-dihydro-4H- 1,2,4-triazol-4-yl)pyridin- 2-yl)-1-isopropyl-7- methyl-N-(4-(3- methylureido)benzyl)- 4,5,6,7-tetrahydro-1H- pyrazolo[4,3-c]pyridine- 3-carboxamide | 573.5 |
| 522 | Ex. 2^{l} | 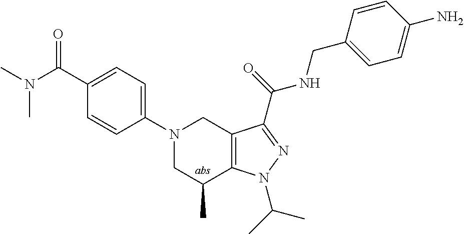 [SMILES: CC(C)n1nc(C(=O)NCc2ccc(N)cc2)c2c1[C@@H](C)CN(c1ccc(C(=O)N(C)C)cc1)C2] | (S)-N-(4-aminobenzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 475.3 |
| 523 | Ex. 523 | 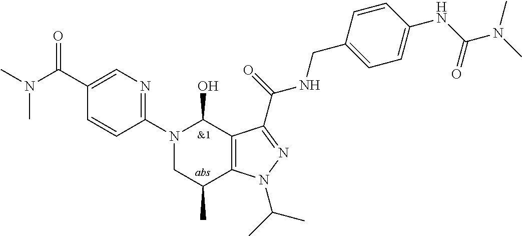 ¹H NMR (600 MHz, (CD₃)₂SO) δ 8.61 (t, 1H), 8.28-8.22 (m, 2H), 7.71-7.63 (m, 1H), 7.44-7.38 (m, 2H), 7.23-7.16 (m, 2H), 7.10-7.01 (m, 1H), 6.62 (s, 1H), 5.98- 5.95 (m, 1H), 4.69-4.64 (m, 1H), 4.58- 4.51 (m, 1H), 4.46-4.34 (m, 2H), 3.49- 3.43 (m, 1H), 3.22-3.19 (m, 1H), 3.03- 2.95 (m, 6H), 2.92 (s, 6H), 1.50-1.34 (m, 6H), 1.14-1.05 (m, 3H). | (4RS,7S)-5-(5- (dimethylcarbamoyl) pyridin-2-yl)-N-(4-(3,3- dimethylureido)benzyl)- 4-hydroxy-1-isopropyl-7- methyl-4,5,6,7- tetrahydro-1H- pyrazolo[4,3-c]pyridine- 3-carboxamide | |
| 524 | Ex. 524 | 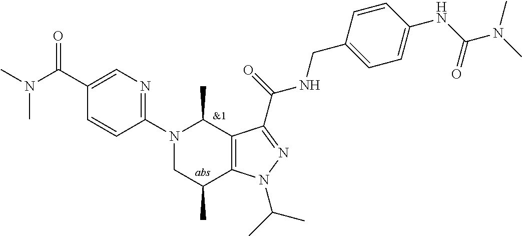 ¹H NMR (600 MHz, (CD₃)₂SO) δ 8.34 (t, 1H), 8.24-8.19 (m, 2H), 7.64 (d, 1H), 7.41- 7.36 (m, 2H), 7.20-7.15 (m, 2H), 6.93 (d, 1H), 5.73 (s, 1H), 4.52-4.46 (m, 2H), 4.43- 4.37 (m, 1H), 4.34-4.27 (m, 1H), 3.42 (d, 1H), 3.23-3.16 (m, 1H), 2.98 (s, 6H), 2.90 (s, 6H), 1.43-1.36 (m, 9H), 1.13 (d, 3H). | (4RS,7S)-5-(5- (dimethylcarbamoyl) pyridin-2-yl)-N-(4-(3,3- dimethylureido)benzyl)- 1-isopropyl-4,7- dimethyl-4,5,6,7- tetrahydro-1H- pyrazolo[4,3-c]pyridine- 3-carboxamide | 561.4 |
| 525 | Ex. 485 | 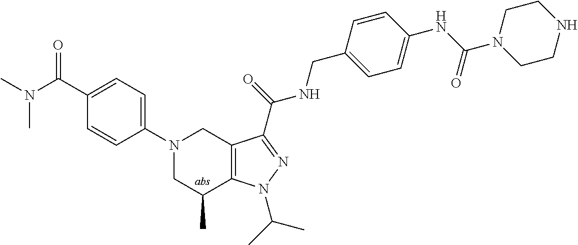 [SMILES: CC(C)n1nc(C(=O)NCc2ccc(NC(=O)N3CCNCC3)cc2)c2c1[C@@H](C)CN(c1ccc(C(=O)N(C)C)cc1)C2] | (S)-5-(4- (dimethylcarbamoyl) phenyl)-1-isopropyl-7- methyl-N-(4-(piperazine- 1-carboxamido)benzyl)- 4,5,6,7-tetrahydro-1H- pyrazolo[4,3-c]pyridine- 3-carboxamide | 587.4 |
| 526 | Ex. 113 | 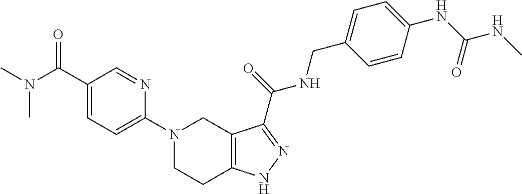 | 5-(5- (dimethylcarbamoyl) pyridin-2-yl)-N-(4-(3- methylureido)benzyl)- 4,5,6,7-tetrahydro-1H- pyrazolo[4,3-c]pyridine- 3-carboxamide | 477.1 |
| 527^{∧} | Ex. 537 | 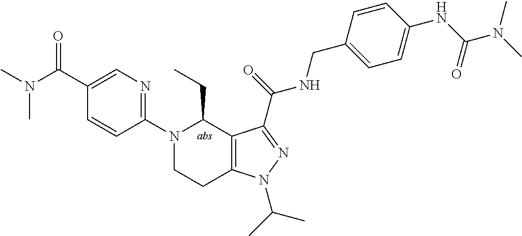 | (S)-5-(5- (dimethylcarbamoyl) pyridin-2-yl)-N-(4-(3,3- dimethylureido)benzyl)- 4-ethyl-1-isopropyl- 4,5,6,7-tetrahydro-1H- pyrazolo[4,3-c]pyridine- 3-carboxamide | 561.4 |
| 528^{∧} | Ex. 537 | 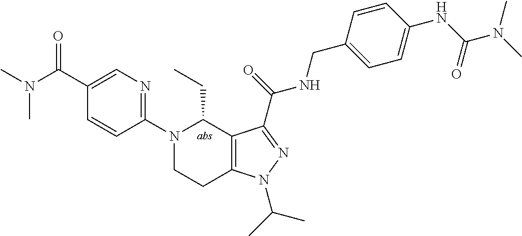 | (R)-5-(5- (dimethylcarbamoyl) pyridin-2-yl)-N-(4-(3,3- dimethylureido)benzyl)- 4-ethyl-1-isopropyl- 4,5,6,7-tetrahydro-1H- pyrazolo[4,3-c]pyridine- 3-carboxamide | 561.4 |
| 529^{∧} | Ex. 529 | 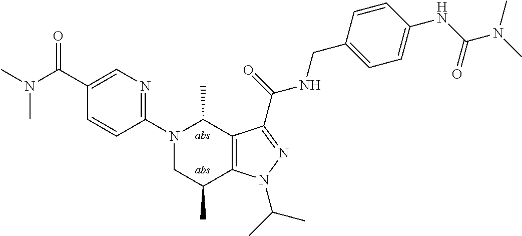 ¹H NMR (600 MHz, (CD₃)₂SO) δ 8.29 (t, 1H), 8.21-8.17 (m, 2H), 7.57 (dd, 1H), 7.37-7.31 (m, 2H), 7.18-7.10 (m, 2H), 6.83 (d, 1H), 5.70 (s, 1H), 4.50-4.40 (m, 2H), 4.39-4.30 (m, 1H), 4.29-4.20 (m, 1H), 3.31-3.27 (m, 1H), 3.18-3.08 (m, 1H), 2.94 (s, 6H), 2.86 (s, 6H), 1.39-1.27 (m, 9H), 1.08 (d, 3H). | (4R,7S)-5-(5- (dimethylcarbamoyl) pyridin-2-yl)-N-(4-(3,3- dimethylureido)benzyl)- 1-isopropyl-4,7- dimethyl-4,5,6,7- tetrahydro-1H- pyrazolo[4,3-c]pyridine- 3-carboxamide | 561.2 |
| 530 | Ex. 530 | 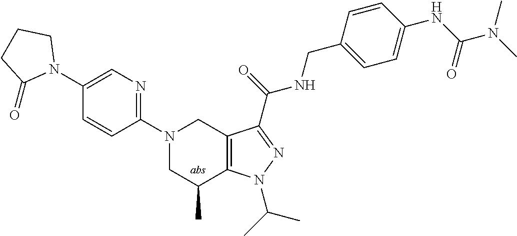 [SMILES: CC(C)n1nc(C(=O)NCc2ccc(NC(=O)N(C)C)cc2)c2c1[C@@H](C)CN(c1ccc(N3CCCC3=O)cn1)C2] ¹H NMR (400 MHz, (CD₃)₂SO) δ 8.33 (t, 1H), 8.29 (d, 1H), 8.23 (s, 1H), 7.88 (dd, 1H), 7.43-7.35 (m, 2H), 7.21-7.15 (m, 2H), 6.90 (d, 1H), 4.95 (d, 1H), 4.59-4.48 (m, 1H), 4.40-4.28 (m, 3H), 4.17 (d, 1H), 3.78 (t, 2H), 3.32-3.18 (m, 2H), 2.91 (s, 6H), 2.44 (t, 2H), 2.12-2.00 (m, 2H), 1.47- 1.38 (m, 6H), 1.21 (d, 3H). | (S)-N-(4-(3,3- dimethylureido)benzyl)- 1-isopropyl-7-methyl-5- (5-(2-oxopyrrolidin-1- yl)pyridin-2-yl)-4,5,6,7- tetrahydro-1H- pyrazolo[4,3-c]pyridine- 3-carboxamide | 559.2 |
| 531 | Ex. 125 | 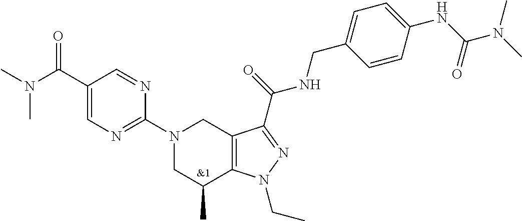 | rac-(R)-5-(5- (dimethylcarbamoyl) pyrimidin-2-yl)-N-(4-(3,3- dimethylureido)benzyl)- 1-ethyl-7-methyl-4,5,6,7- tetrahydro-1H- pyrazolo[4,3-c]pyridine- 3-carboxamide | 534.5 |
| 532 | Ex. 125 | 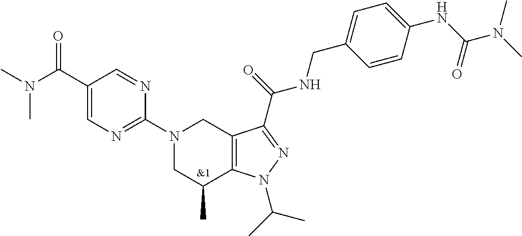 | rac-(R)-5-(5- (dimethylcarbamoyl) pyrimidin-2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 548.6 |
| 533 | Ex. 125 | 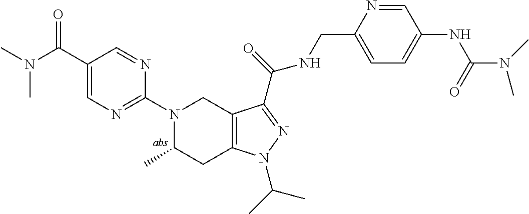 | (S)-5-(5- (dimethylcarbamoyl) pyrimidin-2-yl)-N-((5-(3,3- dimethylureido)pyridin- 2-yl)methyl)-1-isopropyl- 6-methyl-4,5,6,7- tetrahydro-1H- pyrazolo[4,3-c]pyridine- 3-carboxamide | 549.7 |
| 534 | Ex. 125^{e} | 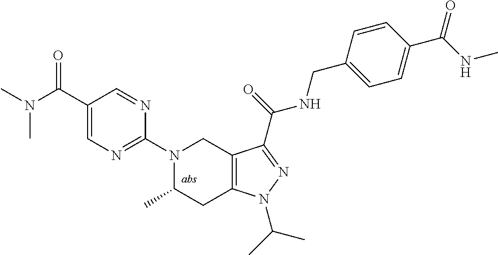 | (S)-5-(5- (dimethylcarbamoyl) pyrimidin-2-yl)-1-isopropyl- 6-methyl-N-(4- (methylcarbamoyl)benzyl)- 4,5,6,7-tetrahydro-1H- pyrazolo[4,3-c]pyridine- 3-carboxamide | 519.6 |
| 535 | Ex. 125^{e} | 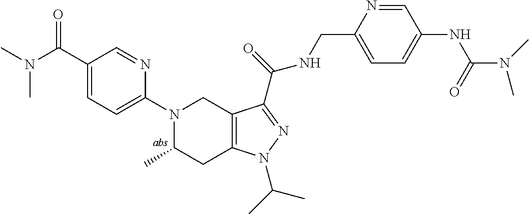 | (S)-5-(5- (dimethylcarbamoyl) pyridin-2-yl)-N-((5-(3,3- dimethylureido)pyridin- 2-yl)methyl)-1-isopropyl- 6-methyl-4,5,6,7- tetrahydro-1H- pyrazolo[4,3-c]pyridine- 3-carboxamide | 548.7 |
| 536 | Ex. 125^{e} | 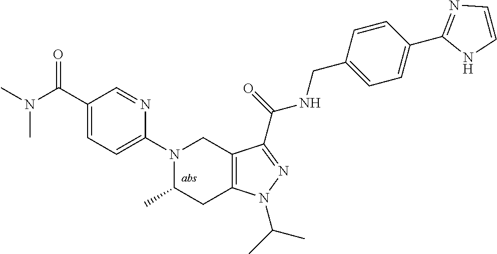 [SMILES: CC(C)n1nc(C(=O)NCc2ccc(-c3ncc[nH]3)cc2)c2c1C[C@H](C)N(c1ccc(C(=O)N(C)C)cn1)C2] | (S)-N-(4-(1H-imidazol-2- yl)benzyl)-5-(5- (dimethylcarbamoyl) pyridin-2-yl)-1-isopropyl-6- methyl-4,5,6,7- tetrahydro-1H- pyrazolo[4,3-c]pyridine- 3-carboxamide | 527.6 |
| 537 | Ex. 537 | 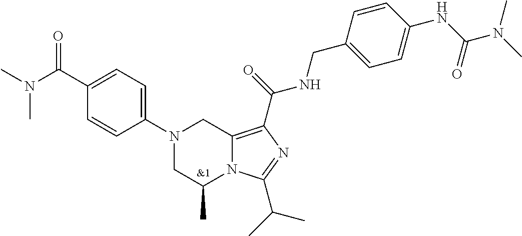 ¹H NMR (400 MHz, (CD₃)₂SO) δ 8.22 (s, 1H), 8.04 (t, 1H), 7.42-7.35 (m, 4H), 7.21- 7.14 (m, 2H), 7.03-6.96 (m, 2H), 4.93 (d, 1H), 4.64-4.58 (m, 1H), 4.43-4.31 (m, 3H), 4.02 (d, 1H), 3.41-3.38 (m, 1H), 3.15- 3.04 (m, 1H), 2.97 (s, 6H), 2.91 (s, 6H), 1.42 (d, 3H), 1.33-1.21 (m, 6H). | rac-(R)-7-(4- (dimethylcarbamoyl) phenyl)-N-(4-(3,3- dimethylureido)benzyl)- 3-isopropyl-5-methyl- 5,6,7,8- tetrahydroimidazo[1,5- a]pyrazine-1- carboxamide | 546.3 |
| 538^{∧} | Ex. 538 | 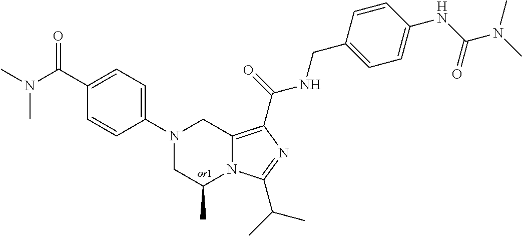 ¹H NMR (400 MHz, (CD₃)₂SO) δ 8.19 (s, 1H), 8.02 (t, 1H), 7.38-7.31 (m, 4H), 7.17- 7.10 (m, 2H), 6.99-6.92 (m, 2H), 4.89 (d, 1H), 4.62-4.52 (m, 1H), 4.40-4.24 (m, 3H), 3.98 (d, 1H), 3.39-3.35 (m, 1H), 3.13- 3.00 (m, 1H), 2.93 (s, 6H), 2.87 (s, 6H), 1.37 (d, 3H), 1.29-1.17 (m, 6H). | rel-(R or S)-7-(4- (dimethylcarbamoyl) phenyl)-N-(4-(3,3- dimethylureido)benzyl)- 3-isopropyl-5-methyl- 5,6,7,8- tetrahydroimidazo[1,5- a]pyrazine-1- carboxamide | 546.3 |
| 539^{∧} | Ex. 539 | 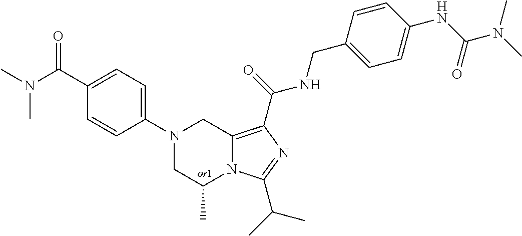 ¹H NMR (400 MHz, (CD₃)₂SO) δ 8.19 (s, 1H), 8.02 (t, 1H), 7.38-7.28 (m, 4H), 7.18- 7.07 (m, 2H), 7.00-6.91 (m, 2H), 4.88 (d, 1H), 4.60-4.52 (m, 1H), 4.38-4.26 (m, 3H), 3.97 (d, 1H), 3.42-3.38 (m, 1H), 3.11- 3.00 (m, 1H), 2.92 (s, 6H), 2.86 (s, 6H), 1.36 (d, 3H), 1.27-1.17 (m, 6H). | rel-(R or S)-7-(4- (dimethylcarbamoyl) phenyl)-N-(4-(3,3- dimethylureido)benzyl)- 3-isopropyl-5-methyl- 5,6,7,8- tetrahydroimidazo[1,5- a]pyrazine-1- carboxamide | 546.3 |
| 540 | Ex. 125^{e} | 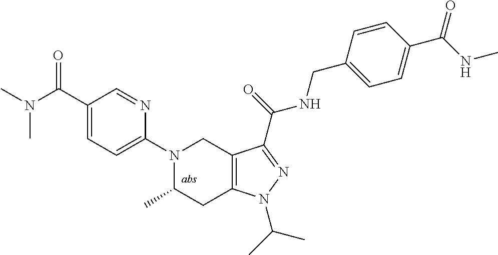 | (S)-5-(5- (dimethylcarbamoyl) pyridin-2-yl)-1-isopropyl-6- methyl-N-(4- (methylcarbamoyl)benzyl)- 4,5,6,7-tetrahydro-1H- pyrazolo[4,3-c]pyridine- 3-carboxamide | 518.6 |
| 541 | Ex. 4^{k} | 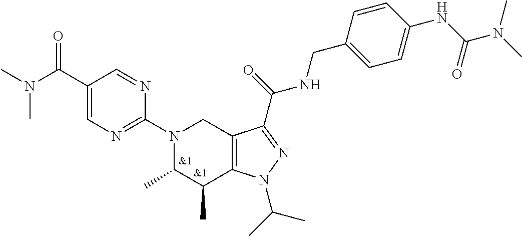 | rac-(6R,7R)-5-(5- (dimethylcarbamoyl) pyrimidin-2-yl)-N-(4-(3,3- dimethylureido)benzyl)- 1-isopropyl-6,7- dimethyl-4,5,6,7- tetrahydro-1H- pyrazolo[4,3-c]pyridine- 3-carboxamide | 562.5 |
| 542 | Ex. 23^{k} | 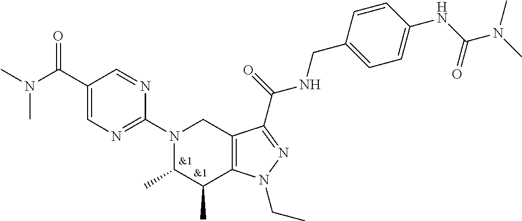 | rac-(6R,7R)-5-(5- (dimethylcarbamoyl) pyrimidin-2-yl)-N-(4-(3,3- dimethylureido)benzyl)- 1-ethyl-6,7-dimethyl- 4,5,6,7-tetrahydro-1H- pyrazolo[4,3-c]pyridine- 3-carboxamide | 548.5 |
| 543 | Ex. 127 | 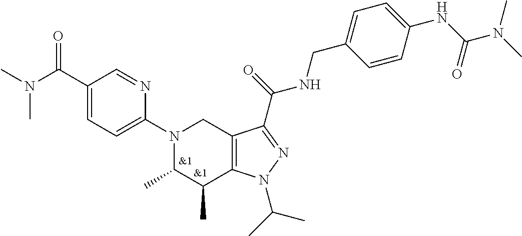 | rac-(6R,7R)-5-(5- (dimethylcarbamoyl) pyridin-2-yl)-N-(4-(3,3- dimethylureido)benzyl)- 1-isopropyl-6,7- dimethyl-4,5,6,7- tetrahydro-1H- pyrazolo[4,3-c]pyridine- 3-carboxamide | 561.5 |
| 544 | Ex. 39^{f} | 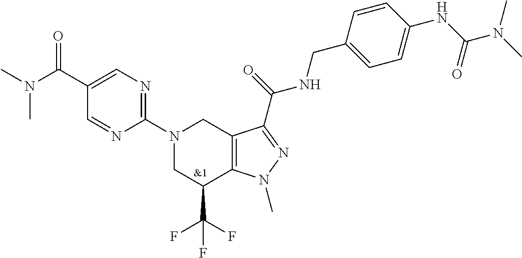 | rac-(R)-5-(5- (dimethylcarbamoyl) pyrimidin-2-yl)-N-(4-(3,3- dimethylureido)benzyl)- 1-methyl-7- (trifluoromethyl)-4,5,6,7- tetrahydro-1H- pyrazolo[4,3-c]pyridine- 3-carboxamide | 574.7 |
| 545 | Ex. 39^{f} | 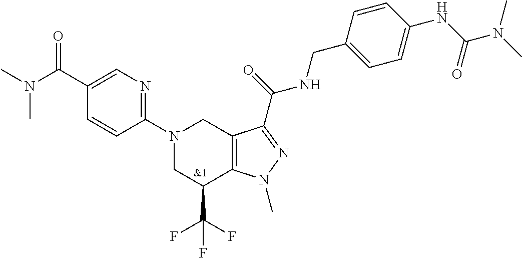 | rac-(R)-5-(5- (dimethylcarbamoyl) pyridin-2-yl)-N-(4-(3,3- dimethylureido)benzyl)- 1-methyl-7- (trifluoromethyl)-4,5,6,7- tetrahydro-1H- pyrazolo[4,3-c]pyridine- 3-carboxamide | 573.6 |
| 546 | Ex. 546 | 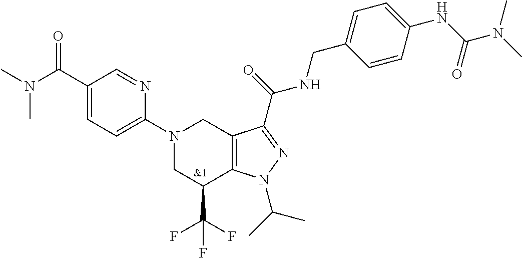 ¹H NMR (600 MHz, (CD₃)₂SO) δ 8.57- 8.52 (m, 1H), 8.29-8.27 (m, 1H), 8.26- 8.22 (m, 1H), 7.72-7.67 (m, 1H), 7.43- 7.38 (m, 2H), 7.23-7.15 (m, 2H), 6.87- 6.82 (m, 1H), 4.72-4.65 (m, 1H), 4.56- 4.50 (m, 1H), 4.41-4.33 (m, 2H), 4.31- 4.26 (m, 1H), 4.13-4.07 (m, 1H), 3.22- 3.15 (m, 2H), 2.99 (s, 6H), 2.93-2.90 (m, 6H), 1.48-1.40 (m, 6H). | rac-(R)-5-(5- (dimethylcarbamoyl) pyridin-2-yl)-N-(4-(3,3- dimethylureido)benzyl)- 1-isopropyl-7- (trifluoromethyl)-4,5,6,7- tetrahydro-1H- pyrazolo[4,3-c]pyridine- 3-carboxamide | 601.7 |
| 547^{∧} | Ex. 547 | 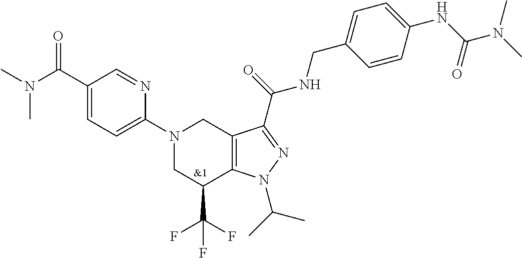 ¹H NMR (600 MHz, (CD₃)₂SO) δ 8.55 (t, 1H), 8.29-8.27 (m, 1H), 8.24 (s, 1H), 7.69 (dd, 1H), 7.42-7.38 (m, 2H), 7.21-7.17 (m, 2H), 6.84 (d, 1H), 4.72-4.65 (m, 1H), 4.57-4.49 (m, 1H), 4.42-4.32 (m, 2H), 4.31-4.25 (m, 1H), 4.16-4.06 (m, 1H), 3.30-3.27 (m, 1H), 3.18-3.15 (m, 1H), 2.99 (s, 6H), 2.91 (s, 6H), 1.44 (dd, 6H). | rel-(R or S)-5-(5- (dimethylcarbamoyl) pyridin-2-yl)-N-(4-(3,3- dimethylureido)benzyl)- 1-isopropyl-7- (trifluoromethyl)-4,5,6,7- tetrahydro-1H- pyrazolo[4,3-c]pyridine- 3-carboxamide | 601.3 |
| 548^{∧} | Ex. 548 | 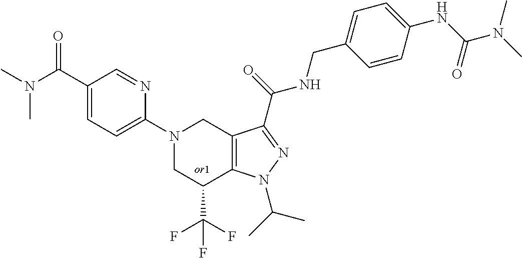 | rel-(R or S)-5-(5- (dimethylcarbamoyl) pyridin-2-yl)-N-(4-(3,3- dimethylureido)benzyl)- 1-isopropyl-7- (trifluoromethyl)-4,5,6,7- tetrahydro-1H- pyrazolo[4,3-c]pyridine- 3-carboxamide | 601.3 |
| 549^{∧} | Ex. 127 | 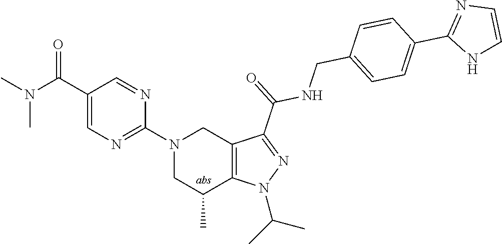 [SMILES: CC(C)n1nc(C(=O)NCc2ccc(-c3ncc[nH]3)cc2)c2c1[C@H](C)CN(c1ncc(C(=O)N(C)C)cn1)C2] | (R)-N-(4-(1H-imidazol-2- yl)benzyl)-5-(5- (dimethylcarbamoyl) pyrimidin-2-yl)-1-isopropyl- 7-methyl-4,5,6,7- tetrahydro-1H- pyrazolo[4,3-c]pyridine- 3-carboxamide | 528.3 |
| 550^{∧} | Ex. 113 | 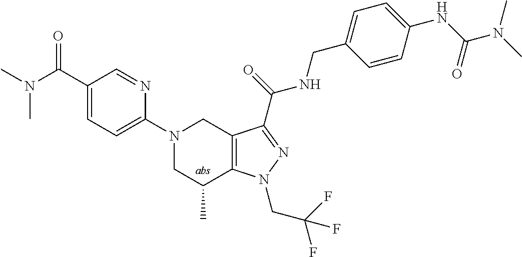 | (R)-5-(5- (dimethylcarbamoyl) pyridin-2-yl)-N-(4-(3,3- dimethylureido)benzyl)- 7-methyl-1-(2,2,2- trifluoroethyl)-4,5,6,7- tetrahydro-1H- pyrazolo[4,3-c]pyridine- 3-carboxamide | 587.3 |
| 551^{∧} | Ex. 113 | 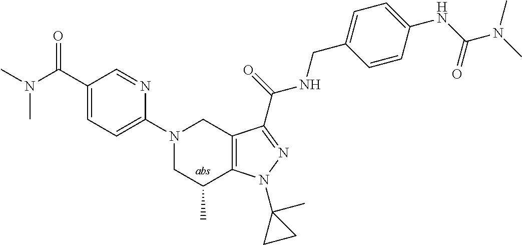 | (R)-5-(5- (dimethylcarbamoyl) pyridin-2-yl)-N-(4-(3,3- dimethylureido)benzyl)- 7-methyl-1-(1- methylcyclopropyl)- 4,5,6,7-tetrahydro-1H- pyrazolo[4,3-c]pyridine- 3-carboxamide | 559.3 |
| 552^{∧} | Ex. 125 | 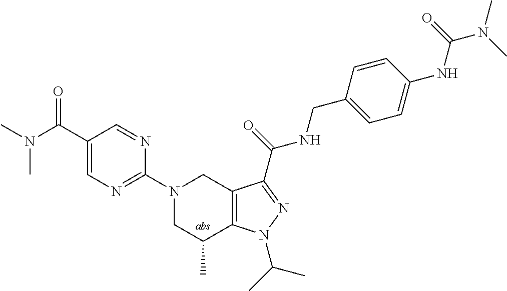 | (R)-5-(5- (dimethylcarbamoyl) pyrimidin-2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 548.3 |
| 553^{∧} | Ex. 113 | 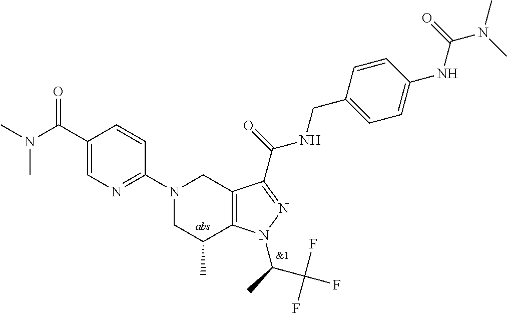 | (R)-5-(5- (dimethylcarbamoyl) pyridin-2-yl)-N-(4-(3,3- dimethylureido)benzyl)- 7-methyl-1-((RS)-1,1,1- trifluoropropan-2-yl)- 4,5,6,7-tetrahydro-1H- pyrazolo[4,3-c]pyridine- 3-carboxamide | 601.3 |
| 554 | Ex. 252 | 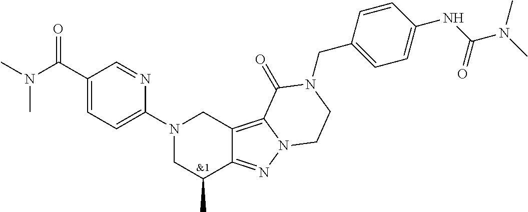 | rac-(R)-6-(9-(4-(3,3- dimethylureido)benzyl)- 4-methyl-10-oxo- 3,4,7,8,9,10- hexahydropyrido[4′,3′:3, 4]pyrazolo[1,5- a]pyrazin-2(1H)-yl)-N,N- dimethylnicotinamide | 531.4 |
| 555 | Ex. 127 | 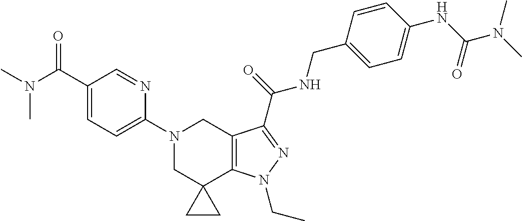 | 5′-(5- (dimethylcarbamoyl) pyridin-2-yl)-N-(4-(3,3- dimethylureido)benzyl)- 1′-ethyl-1′,4′,5′,6′- tetrahydrospiro [cyclopropane-1,7′- pyrazolo[4,3- c]pyridine]-3′- carboxamide | 545.6 |
| 556^{∧} | Ex. 113 | 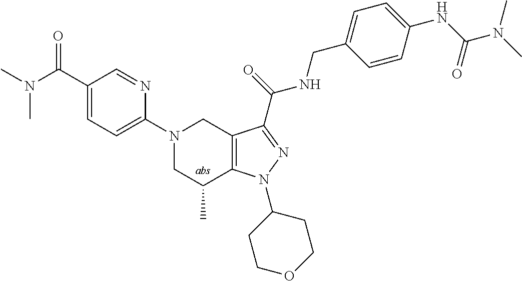 | (R)-5-(5- (dimethylcarbamoyl) pyridin-2-yl)-N-(4-(3,3- dimethylureido)benzyl)- 7-methyl-1-(tetrahydro- 2H-pyran-4-yl)-4,5,6,7- tetrahydro-1H- pyrazolo[4,3-c]pyridine- 3-carboxamide | 589.3 |
| 557^{∧} | Ex. 113 | 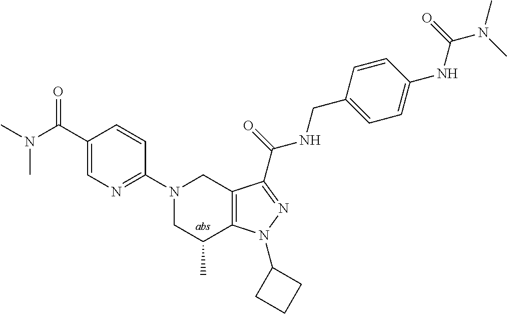 | (R)-1-cyclobutyl-5-(5- (dimethylcarbamoyl) pyridin-2-yl)-N-(4-(3,3- dimethylureido)benzyl)- 7-methyl-4,5,6,7- tetrahydro-1H- pyrazolo[4,3-c]pyridine- 3-carboxamide | 559.3 |
| 558 | Ex. 113^{f} | 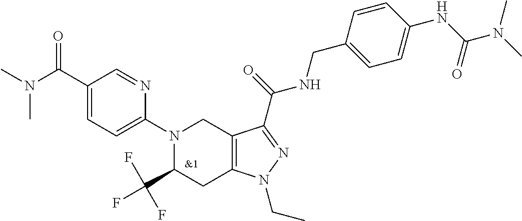 | rac-(R)-5-(5- (dimethylcarbamoyl) pyridin-2-yl)-N-(4-(3,3- dimethylureido)benzyl)- 1-ethyl-6- (trifluoromethyl)-4,5,6,7- tetrahydro-1H- pyrazolo[4,3-c]pyridine- 3-carboxamide | 587.5 |
| 559 | Ex. 561 | 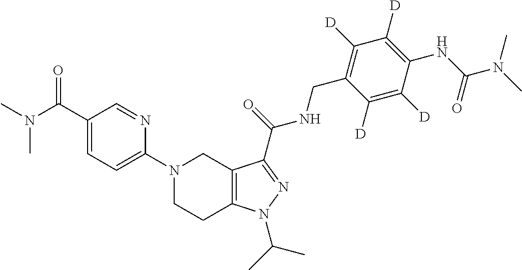 ¹H NMR (400 MHz, (CD₃)₂SO) δ 8.32 (t, 1H), 8.26-8.19 (m, 2H), 7.63 (dd, 1H), 6.88 (d, 1H), 4.75 (s, 2H), 4.55-4.43 (m, 1H), 4.35 (d, 2H), 3.95 (t, 2H), 2.97 (s, 6H), 2.91 (s, 6H), 2.82 (t, 2H), 1.39 (d, 6H) | 5-(5- (dimethylcarbamoyl) pyridin-2-yl)-N-(4-(3,3- dimethylureido) 2,3,5,6- d₄-benzyl)-1-isopropyl- 4,5,6,7-tetrahydro-1H- pyrazolo[4,3-c]pyridine- 3-carboxamide | 537.3 |
| 560 | Ex. 128 | 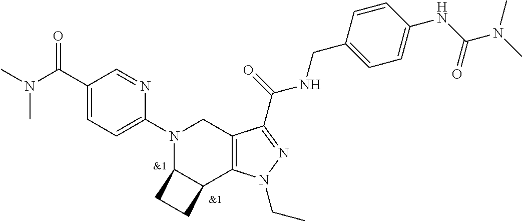 | rac-(5aR,7aS)-5-(5- (dimethylcarbamoyl) pyridin-2-yl)-N-(4-(3,3- dimethylureido)benzyl)- 1-ethyl-4,5,5a,6,7,7a- hexahydro-1H- cyclobuta[b]pyrazolo[3,4- d]pyridine-3- carboxamide | 545.4 |
| 561 | Ex. 136 | 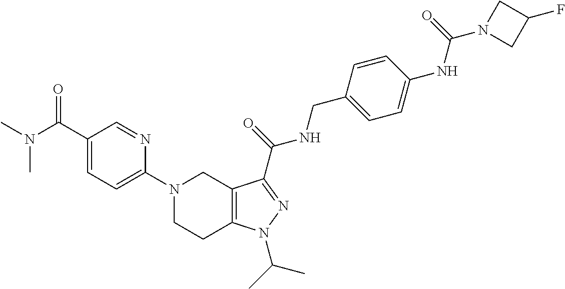 [SMILES: CC(C)n1nc(C(=O)NCc2ccc(NC(=O)N3CC(F)C3)cc2)c2c1CCN(c1ccc(C(=O)N(C)C)cn1)C2] | 5-(5- (dimethylcarbamoyl) pyridin-2-yl)-N-(4-(3- fluoroazetidine-1- carboxamido)benzyl)-1- isopropyl-4,5,6,7- tetrahydro-1H- pyrazolo[4,3-c]pyridine- 3-carboxamide | 563.4 |
| 562 | Ex. 515 | 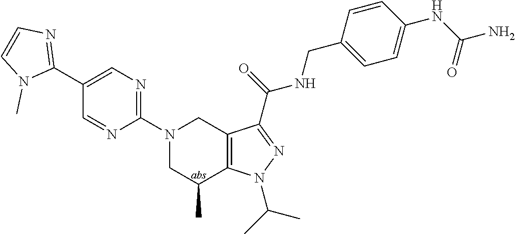 [SMILES: CC(C)n1nc(C(=O)NCc2ccc(NC(N)=O)cc2)c2c1[C@@H](C)CN(c1ncc(-c3nccn3C)cn1)C2] | (S)-1-isopropyl-7- methyl-5-(5-(1-methyl- 1H-imidazol-2- yl)pyrimidin-2-yl)-N-(4- ureidobenzyl)-4,5,6,7- tetrahydro-1H- pyrazolo[4,3-c]pyridine- 3-carboxamide | 529.4 |
| 563 | Ex. 193 | 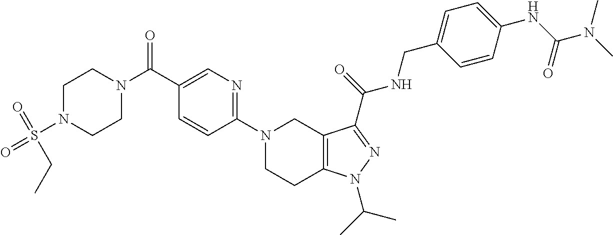 [SMILES: CCS(=O)(=O)N1CCN(C(=O)c2ccc(N3CCc4c(c(C(=O)NCc5ccc(NC(=O)N(C)C)cc5)nn4C(C)C)C3)nc2)CC1] | N-(4-(3,3- dimethylureido)benzyl)- 5-(5-(4- (ethylsulfonyl)piperazine- 1-carbonyl)pyridin-2-yl)- 1-isopropyl-4,5,6,7- tetrahydro-1H- pyrazolo[4,3-c]pyridine- 3-carboxamide | 666.4 |
| 564 | Ex. 193 | 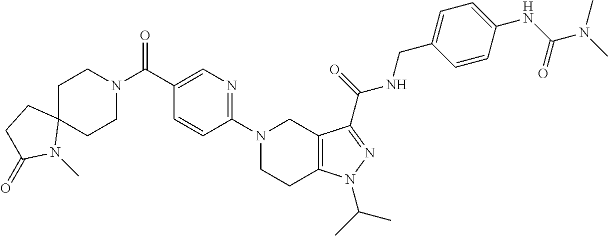 [SMILES: CC(C)n1nc(C(=O)NCc2ccc(NC(=O)N(C)C)cc2)c2c1CCN(c1ccc(C(=O)N3CCC4(CCC(=O)N4C)CC3)cn1)C2] | N-(4-(3,3- dimethylureido)benzyl)- 1-isopropyl-5-(5-(1- methyl-2-oxo-1,8- diazaspiro[4.5]decane- 8-carbonyl)pyridin-2-yl)- 4,5,6,7-tetrahydro-1H- pyrazolo[4,3-c]pyridine- 3-carboxamide | 656.7 |
| 565 | Ex. 395^{fo} | 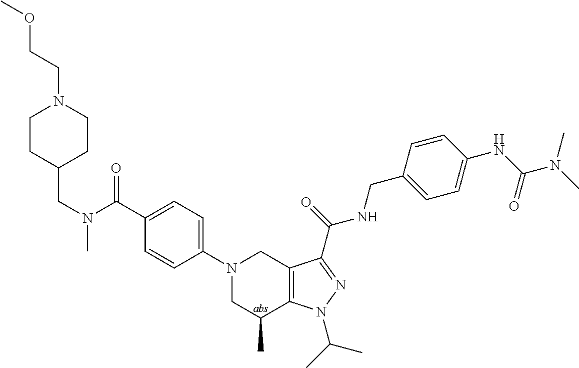 | (S)-N-(4-(3,3- dimethylureido)benzyl)- 1-isopropyl-5-(4-(((1-(2- methoxyethyl)piperidin- 4- yl)methyl)(methyl) carbamoyl)phenyl)-7-methyl- 4,5,6,7-tetrahydro-1H- pyrazolo[4,3-c]pyridine- 3-carboxamide | 688.3 |
| 566 | Ex. 485 | 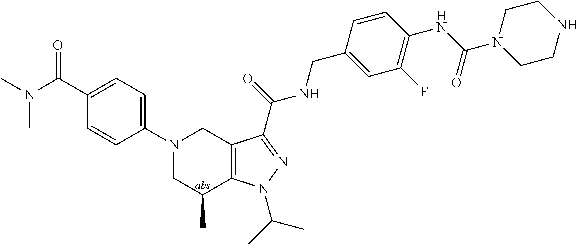 [SMILES: CC(C)n1nc(C(=O)NCc2ccc(NC(=O)N3CCNCC3)c(F)c2)c2c1[C@@H](C)CN(c1ccc(C(=O)N(C)C)cc1)C2] | (S)-5-(4- (dimethylcarbamoyl) phenyl)-N-(3-fluoro-4- (piperazine-1- carboxamido)benzyl)-1- isopropyl-7-methyl- 4,5,6,7-tetrahydro-1H- pyrazolo[4,3-c]pyridine- 3-carboxamide | 605.4 |
| 567 | Ex. 567 | 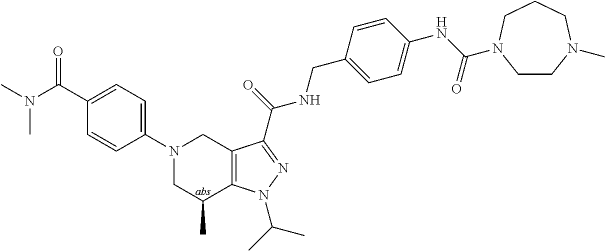 | (S)-5-(4- (dimethylcarbamoyl) phenyl)-1-isopropyl-7- methyl-N-(4-(4-methyl- 1,4-diazepane-1- carboxamido)benzyl)- 4,5,6,7-tetrahydro-1H- pyrazolo[4,3-c]pyridine- 3-carboxamide | 615.6 |
| 568 | Ex. 485 | 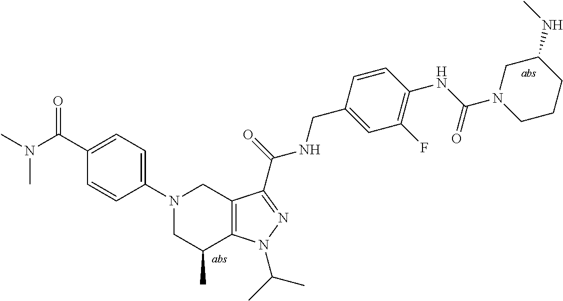 [SMILES: CN[C@@H]1CCCN(C(=O)Nc2ccc(CNC(=O)c3nn(C(C)C)c4c3CN(c3ccc(C(=O)N(C)C)cc3)C[C@@H]4C)cc2F)C1] | (S)-5-(4- (dimethylcarbamoyl) phenyl)-N-(3-fluoro-4-((R)-3- (methylamino)piperidine- 1-carboxamido)benzyl)- 1-isopropyl-7-methyl- 4,5,6,7-tetrahydro-1H- pyrazolo[4,3-c]pyridine- 3-carboxamide | 633.5 |
| 569 | Ex. 485 | 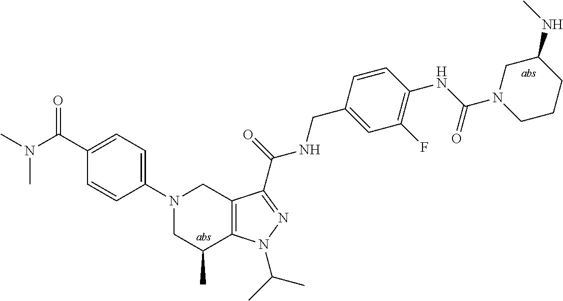 [SMILES: CN[C@H]1CCCN(C(=O)Nc2ccc(CNC(=O)c3nn(C(C)C)c4c3CN(c3ccc(C(=O)N(C)C)cc3)C[C@@H]4C)cc2F)C1] | (S)-5-(4- (dimethylcarbamoyl) phenyl)-N-(3-fluoro-4-((S)-3- (methylamino)piperidine- 1-carboxamido)benzyl)- 1-isopropyl-7-methyl- 4,5,6,7-tetrahydro-1H- pyrazolo[4,3-c]pyridine- 3-carboxamide | 633.5 |
| 570 | Ex. 485 | 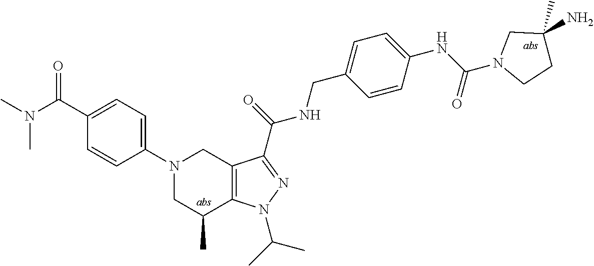 [SMILES: CC(C)n1nc(C(=O)NCc2ccc(NC(=O)N3CC[C@](C)(N)C3)cc2)c2c1[C@@H](C)CN(c1ccc(C(=O)N(C)C)cc1)C2] | (S)-N-(4-((S)-3-amino-3- methylpyrrolidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 601.5 |
| 571 | Ex. 485 | 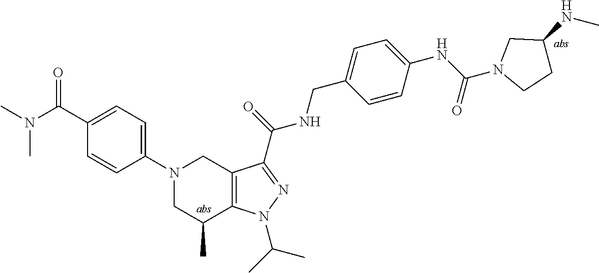 | (S)-5-(4- (dimethylcarbamoyl) phenyl)-1-isopropyl-7- methyl-N-(4-((S)-3- (methylamino)pyrrolidine- 1-carboxamido)benzyl)- 4,5,6,7-tetrahydro-1H- pyrazolo[4,3-c]pyridine- 3-carboxamide | 601.5 |
| 572 | Ex. 485 | 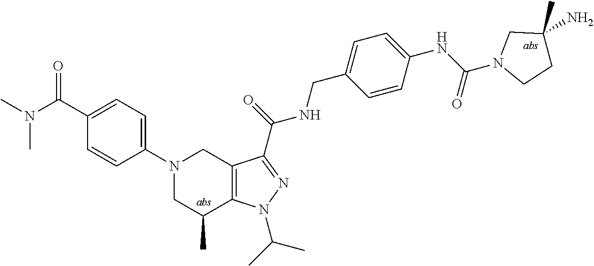 [SMILES: CC(C)n1nc(C(=O)NCc2ccc(NC(=O)N3CC[C@@](C)(N)C3)cc2)c2c1[C@@H](C)CN(c1ccc(C(=O)N(C)C)cc1)C2] | (S)-N-(4-((R)-3-amino-3- methylpyrrolidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 601.4 |
| 573 | Ex. 485 | 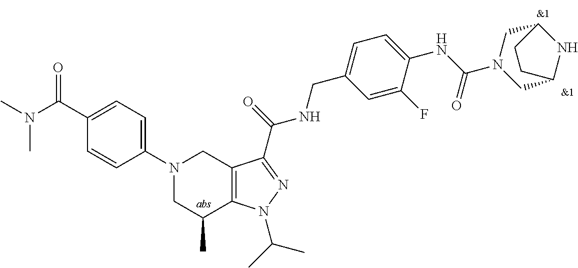 [SMILES: CC(C)n1nc(C(=O)NCc2ccc(NC(=O)N3C[C@H]4CC[C@@H](C3)N4)c(F)c2)c2c1[C@@H](C)CN(c1ccc(C(=O)N(C)C)cc1)C2] | (S)-N-(4-((1RS,5SR)- 3,8- diazabicyclo[3.2.1]octane- 3-carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 631.4 |
| 574 | Ex. 485 | 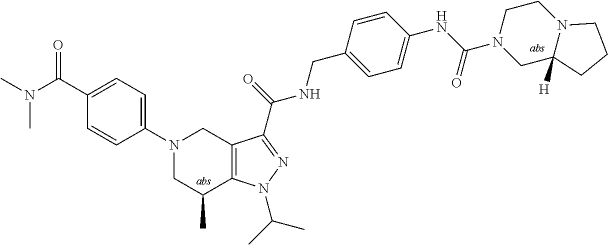 [SMILES: CC(C)n1nc(C(=O)NCc2ccc(NC(=O)N3CCN4CCC[C@@H]4C3)cc2)c2c1[C@@H](C)CN(c1ccc(C(=O)N(C)C)cc1)C2] | (S)-5-(4- (dimethylcarbamoyl) phenyl)-1-isopropyl-7- methyl-N-(4-((R)- octahydropyrrolo[1,2- a]pyrazine-2- carboxamido)benzyl)- 4,5,6,7-tetrahydro-1H- pyrazolo[4,3-c]pyridine- 3-carboxamide | 627.5 |
| 575 | Ex. 485 | 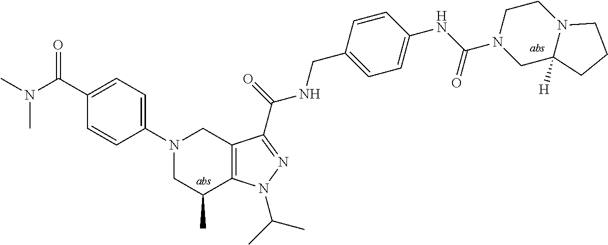 [SMILES: CC(C)n1nc(C(=O)NCc2ccc(NC(=O)N3CCN4CCC[C@H]4C3)cc2)c2c1[C@@H](C)CN(c1ccc(C(=O)N(C)C)cc1)C2] | (S)-5-(4- (dimethylcarbamoyl) phenyl)-1-isopropyl-7- methyl-N-(4-((S)- octahydropyrrolo[1,2- a]pyrazine-2- carboxamido)benzyl)- 4,5,6,7-tetrahydro-1H- pyrazolo[4,3-c]pyridine- 3-carboxamide | 627.5 |
| 576 | Ex. 485 | 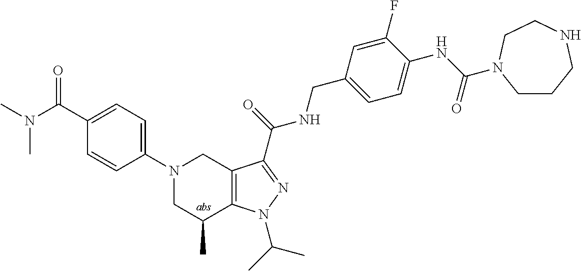 [SMILES: CC(C)n1nc(C(=O)NCc2ccc(NC(=O)N3CCCNCC3)c(F)c2)c2c1[C@@H](C)CN(c1ccc(C(=O)N(C)C)cc1)C2] | (S)-N-(4-(1,4-diazepane- 1-carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 619.5 |
| 577 | Ex. 485 | 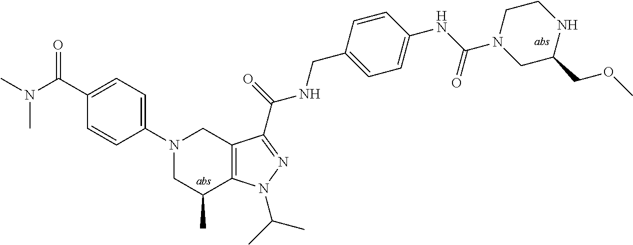 | (S)-5-(4- (dimethylcarbamoyl) phenyl)-1-isopropyl-N-(4- ((R)-3- (methoxymethyl) piperazine-1- carboxamido)benzyl)-7- methyl-4,5,6,7- tetrahydro-1H- pyrazolo[4,3-c]pyridine- 3-carboxamide | 631.5 |
| 578 | Ex. 485 | 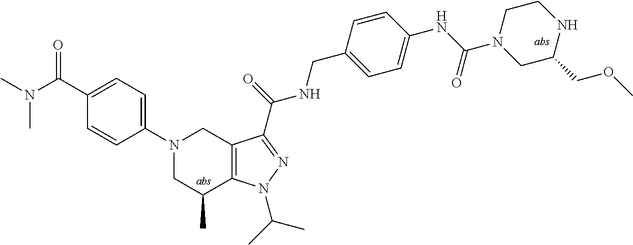 | (S)-5-(4- (dimethylcarbamoyl) phenyl)-1-isopropyl-N-(4- ((S)-3- (methoxymethyl) piperazine-1- carboxamido)benzyl)-7- methyl-4,5,6,7- tetrahydro-1H- pyrazolo[4,3-c]pyridine- 3-carboxamide | 631.5 |
| 579 | Ex. 567 | 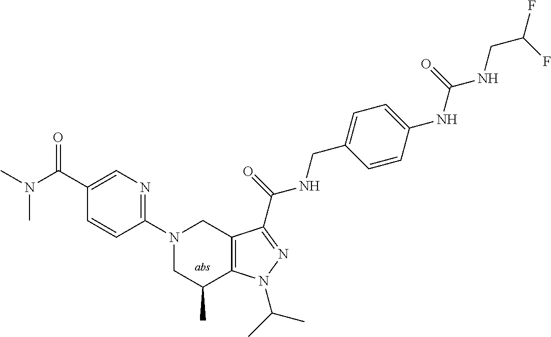 [SMILES: CC(C)n1nc(C(=O)NCc2ccc(NC(=O)NCC(F)F)cc2)c2c1[C@@H](C)CN(c1ccc(C(=O)N(C)C)cn1)C2] | (S)-N-(4-(3-(2,2- difluoroethyl)ureido) benzyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 583.4 |
| 580 | Ex. 567 | 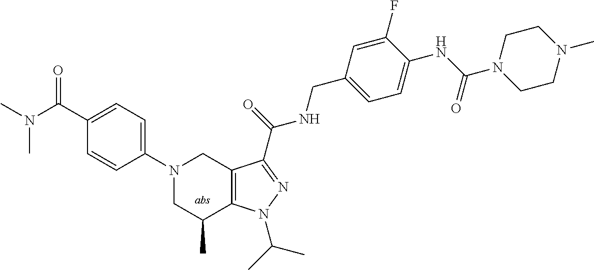 | (S)-5-(4- (dimethylcarbamoyl) phenyl)-N-(3-fluoro-4-(4- methylpiperazine-1- carboxamido)benzyl)-1- isopropyl-7-methyl- 4,5,6,7-tetrahydro-1H- pyrazolo[4,3-c]pyridine- 3-carboxamide | 619.4 |
| 581 | Ex. 485 | 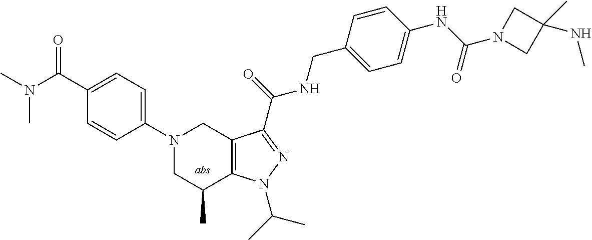 | (S)-5-(4- (dimethylcarbamoyl) phenyl)-1-isopropyl-7- methyl-N-(4-(3-methyl-3- (methylamino)azetidine- 1-carboxamido)benzyl)- 4,5,6,7-tetrahydro-1H- pyrazolo[4,3-c]pyridine- 3-carboxamide | 601.4 |
| 582 | Ex. 485 | 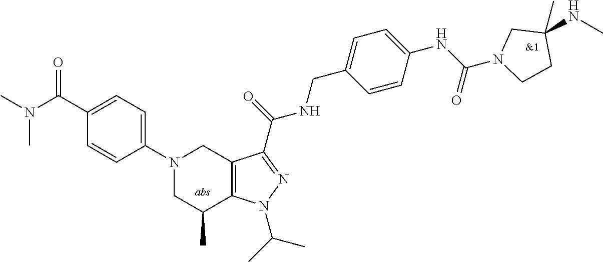 | (S)-5-(4- (dimethylcarbamoyl) phenyl)-1-isopropyl-7- methyl-N-(4-((RS)-3- methyl-3- (methylamino)pyrrolidine- 1-carboxamido)benzyl)- 4,5,6,7-tetrahydro-1H- pyrazolo[4,3-c]pyridine- 3-carboxamide | 615.4 |
| 583 | Ex. 485 | 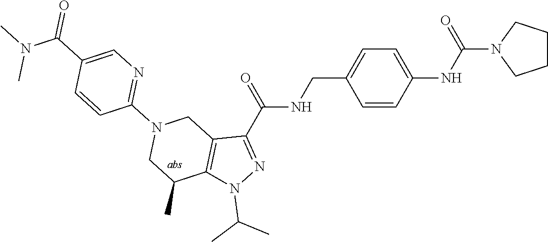 [SMILES: CC(C)n1nc(C(=O)NCc2ccc(NC(=O)N3CCCC3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(pyrrolidine- 1-carboxamido)benzyl)- 4,5,6,7-tetrahydro-1H- pyrazolo[4,3-c]pyridine- 3-carboxamide | 573.4 |
| 584 | Ex. 567 | 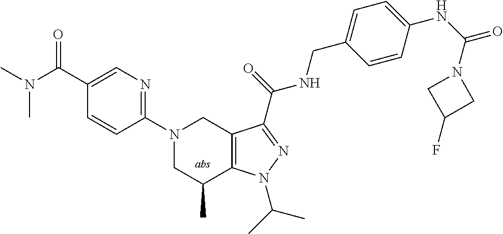 [SMILES: CC(C)n1nc(C(=O)NCc2ccc(NC(=O)N3CC(F)C3)cc2)c2c1[C@@H](C)CN(c1ccc(C(=O)N(C)C)cn1)C2] | (S)-5-(5- (dimethylcarbamoyl) pyridin-2-yl)-N-(4-(3- fluoroazetidine-1- carboxamido)benzyl)-1- isopropyl-7-methyl- 4,5,6,7-tetrahydro-1H- pyrazolo[4,3-c]pyridine- 3-carboxamide | 577.4 |
| 585 | Ex. 567 | 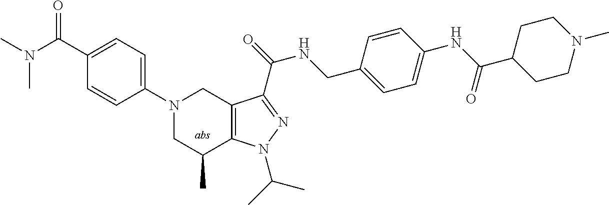 | (S)-5-(4- (dimethylcarbamoyl) phenyl)-1-isopropyl-7- methyl-N-(4-(1- methylpiperidine-4- carboxamido)benzyl)- 4,5,6,7-tetrahydro-1H- pyrazolo[4,3-c]pyridine- 3-carboxamide | 600.5 |
| 586 | Ex. 567 | 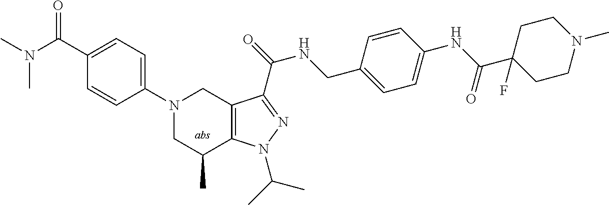 | (S)-5-(4- (dimethylcarbamoyl) phenyl)-N-(4-(4-fluoro-1- methylpiperidine-4- carboxamido)benzyl)-1- isopropyl-7-methyl- 4,5,6,7-tetrahydro-1H- pyrazolo[4,3-c]pyridine- 3-carboxamide | 618.5 |
| 587 | Ex. 128^{s} | 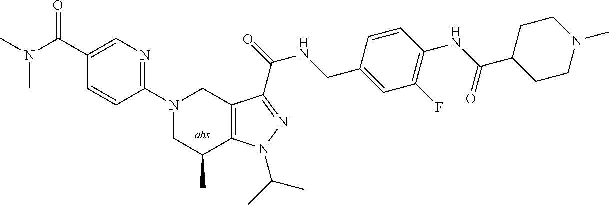 | (S)-5-(5- (dimethylcarbamoyl) pyridin-2-yl)-N-(3-fluoro-4- (1-methylpiperidine-4- carboxamido)benzyl)-1- isopropyl-7-methyl- 4,5,6,7-tetrahydro-1H- pyrazolo[4,3-c]pyridine- 3-carboxamide | 619.5 |
| 588 | Ex. 485 | 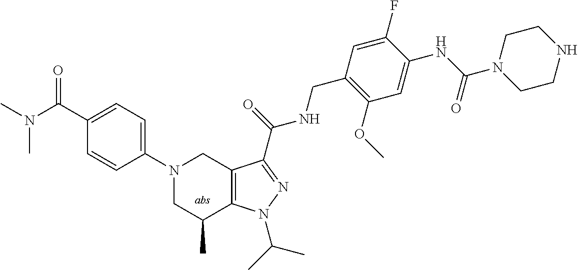 [SMILES: COc1cc(NC(=O)N2CCNCC2)c(F)cc1CNC(=O)c1nn(C(C)C)c2c1CN(c1ccc(C(=O)N(C)C)cc1)C[C@@H]2C] | (S)-5-(4- (dimethylcarbamoyl) phenyl)-N-(5-fluoro-2- methoxy-4-(piperazine- 1-carboxamido)benzyl)- 1-isopropyl-7-methyl- 4,5,6,7-tetrahydro-1H- pyrazolo[4,3-c]pyridine- 3-carboxamide | 635.4 |
| 589 | Ex. 485 | 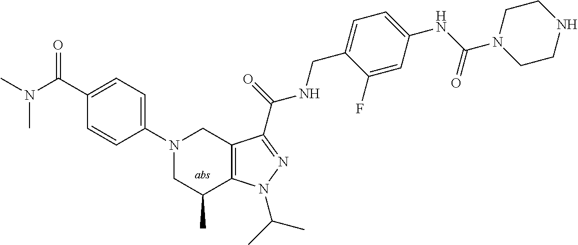 [SMILES: CC(C)n1nc(C(=O)NCc2ccc(NC(=O)N3CCNCC3)cc2F)c2c1[C@@H](C)CN(c1ccc(C(=O)N(C)C)cc1)C2] | (S)-5-(4- (dimethylcarbamoyl) phenyl)-N-(2-fluoro-4- (piperazine-1- carboxamido)benzyl)-1- isopropyl-7-methyl- 4,5,6,7-tetrahydro-1H- pyrazolo[4,3-c]pyridine- 3-carboxamide | 605.5 |
| 590 | Ex. 567 | 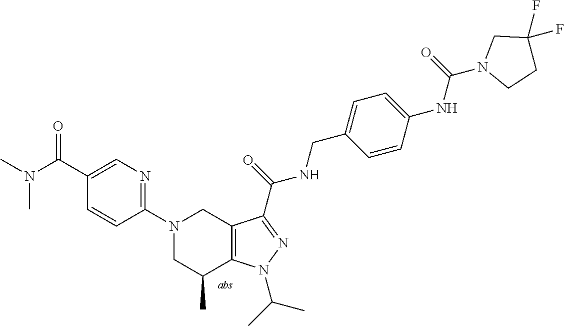 [SMILES: CC(C)n1nc(C(=O)NCc2ccc(NC(=O)N3CCC(F)(F)C3)cc2)c2c1[C@@H](C)CN(c1ccc(C(=O)N(C)C)cn1)C2] | (S)-N-(4-(3,3- difluoropyrrolidine-1- carboxamido)benzyl)-5- (5- (dimethylcarbamoyl) pyridin-2-yl)-1-isopropyl-7- methyl-4,5,6,7- tetrahydro-1H- pyrazolo[4,3-c]pyridine- 3-carboxamide | 609.5 |
| 591 | Ex. 567 | 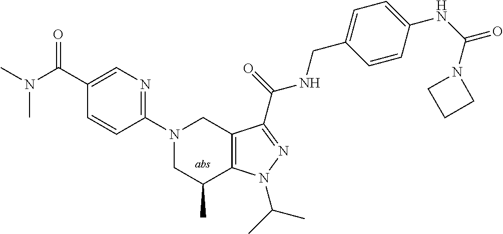 [SMILES: CC(C)n1nc(C(=O)NCc2ccc(NC(=O)N3CCC3)cc2)c2c1[C@@H](C)CN(c1ccc(C(=O)N(C)C)cn1)C2] | (S)-N-(4-(azetidine-1- carboxamido)benzyl)-5- (5- (dimethylcarbamoyl) pyridin-2-yl)-1-isopropyl-7- methyl-4,5,6,7- tetrahydro-1H- pyrazolo[4,3-c]pyridine- 3-carboxamide | 559.3 |
| 592 | Ex. 567 | 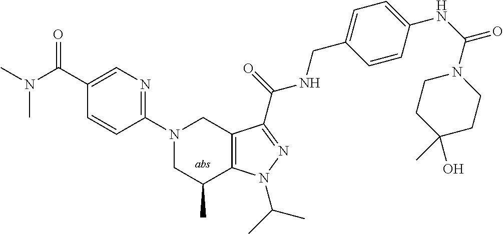 [SMILES: CC(C)n1nc(C(=O)NCc2ccc(NC(=O)N3CCC(C)(O)CC3)cc2)c2c1[C@@H](C)CN(c1ccc(C(=O)N(C)C)cn1)C2] | (S)-5-(5- (dimethylcarbamoyl) pyridin-2-yl)-N-(4-(4- hydroxy-4- methylpiperidine-1- carboxamido)benzyl)-1- isopropyl-7-methyl- 4,5,6,7-tetrahydro-1H- pyrazolo[4,3-c]pyridine- 3-carboxamide | 617.4 |
| 593 | Ex. 567 | 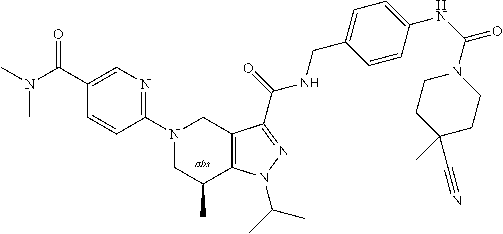 [SMILES: CC(C)n1nc(C(=O)NCc2ccc(NC(=O)N3CCC(C)(C#N)CC3)cc2)c2c1[C@@H](C)CN(c1ccc(C(=O)N(C)C)cn1)C2] | (S)-N-(4-(4-cyano-4- methylpiperidine-1- carboxamido)benzyl)-5- (5- (dimethylcarbamoyl) pyridin-2-yl)-1-isopropyl-7- methyl-4,5,6,7- tetrahydro-1H- pyrazolo[4,3-c]pyridine- 3-carboxamide | 626.5 |
| 594 | Ex. 485 | 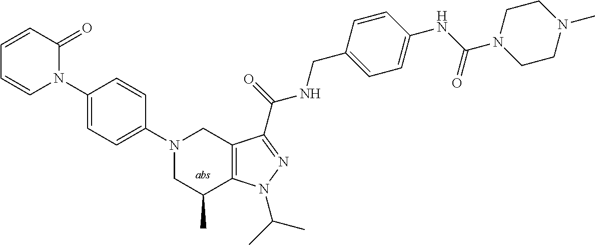 [SMILES: CC(C)n1nc(C(=O)NCc2ccc(NC(=O)N3CCN(C)CC3)cc2)c2c1[C@@H](C)CN(c1ccc(-n3ccccc3=O)cc1)C2] | (S)-1-isopropyl-7- methyl-N-(4-(4- methylpiperazine-1- carboxamido)benzyl)-5- (4-(2-oxopyridin-1(2H)- yl)phenyl)-4,5,6,7- tetrahydro-1H- pyrazolo[4,3-c]pyridine- 3-carboxamide | 623.5 |
| 595 | Ex. 485 | 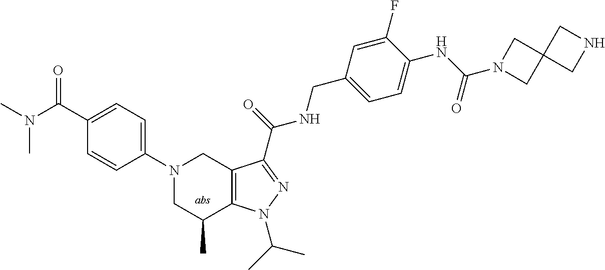 [SMILES: CC(C)n1nc(C(=O)NCc2ccc(NC(=O)N3CC4(CNC4)C3)c(F)c2)c2c1[C@@H](C)CN(c1ccc(C(=O)N(C)C)cc1)C2] | (S)-5-(4- (dimethylcarbamoyl) phenyl)-N-(3-fluoro-4-(2,6- diazaspiro[3.3]heptane- 2-carboxamido)benzyl)- 1-isopropyl-7-methyl- 4,5,6,7-tetrahydro-1H- pyrazolo[4,3-c]pyridine- 3-carboxamide | 617.7 |
| 596 | Ex. 485 | 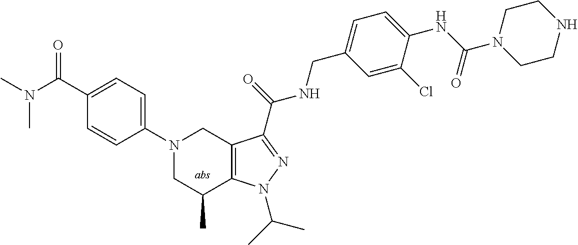 [SMILES: CC(C)n1nc(C(=O)NCc2ccc(NC(=O)N3CCNCC3)c(Cl)c2)c2c1[C@@H](C)CN(c1ccc(C(=O)N(C)C)cc1)C2] | (S)-N-(3-chloro-4- (piperazine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 621.3 |
| 597 | Ex. 485 | 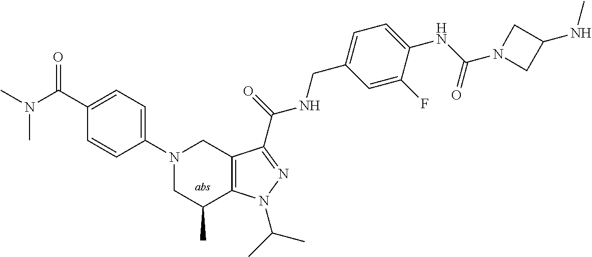 | (S)-5-(4- (dimethylcarbamoyl) phenyl)-N-(3-fluoro-4-(3- (methylamino)azetidine- 1-carboxamido)benzyl)- 1-isopropyl-7-methyl- 4,5,6,7-tetrahydro-1H- pyrazolo[4,3-c]pyridine- 3-carboxamide | 605.6 |
| 598 | Ex. 567 | 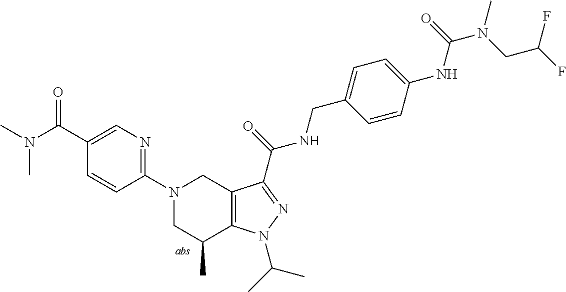 [SMILES: CC(C)n1nc(C(=O)NCc2ccc(NC(=O)N(C)CC(F)F)cc2)c2c1[C@@H](C)CN(c1ccc(C(=O)N(C)C)cn1)C2] | (S)-N-(4-(3-(2,2- difluoroethyl)-3- methylureido)benzyl)-5- (5- (dimethylcarbamoyl) pyridin-2-yl)-1-isopropyl-7- methyl-4,5,6,7- tetrahydro-1H- pyrazolo[4,3-c]pyridine- 3-carboxamide | 597.5 |
| 599 | Ex. 567 | 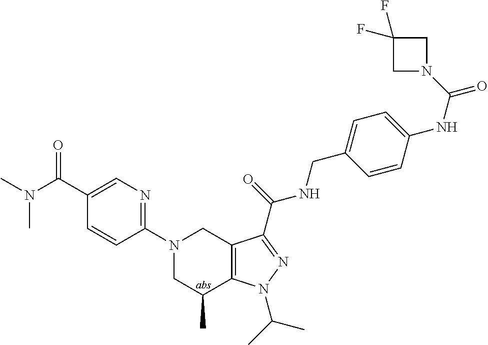 [SMILES: CC(C)n1nc(C(=O)NCc2ccc(NC(=O)N3CC(F)(F)C3)cc2)c2c1[C@@H](C)CN(c1ccc(C(=O)N(C)C)cn1)C2] | (S)-N-(4-(3,3- difluoroazetidine-1- carboxamido)benzyl)-5- (5- (dimethylcarbamoyl) pyridin-2-yl)-1-isopropyl-7- methyl-4,5,6,7- tetrahydro-1H- pyrazolo[4,3-c]pyridine- 3-carboxamide | 595.4 |
| 600 | | 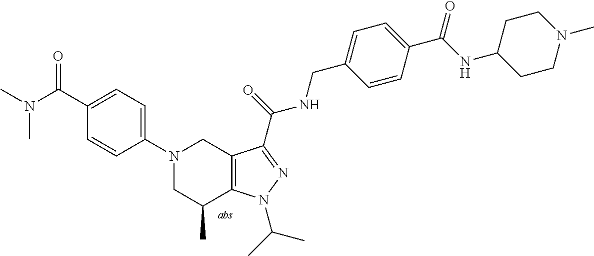 | (S)-5-(4- (dimethylcarbamoyl) phenyl)-1-isopropyl-7- methyl-N-(4-((1- methylpiperidin-4- yl)carbamoyl)benzyl)- 4,5,6,7-tetrahydro-1H- pyrazolo[4,3-c]pyridine- 3-carboxamide | 600.5 |
| 601 | Ex. 132^{ora} | 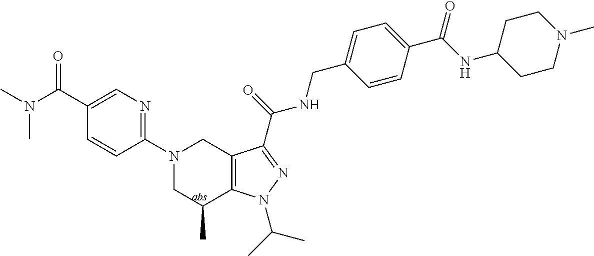 | (S)-5-(5- (dimethylcarbamoyl) pyridin-2-yl)-1-isopropyl-7- methyl-N-(4-((1- methylpiperidin-4- yl)carbamoyl)benzyl)- 4,5,6,7-tetrahydro-1H- pyrazolo[4,3-c]pyridine- 3-carboxamide | 601.5 |
| 602 | Ex 567 | 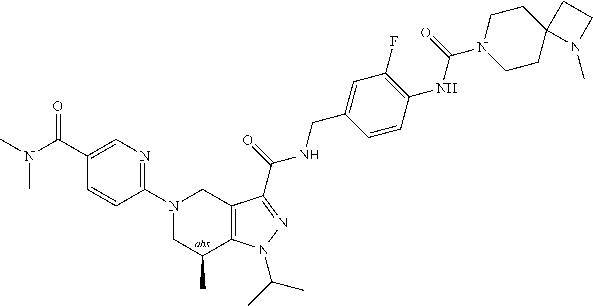 [SMILES: CC(C)n1nc(C(=O)NCc2ccc(NC(=O)N3CCC4(CC3)CCN4C)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- (1-methyl-1,7- diazaspiro[3.5]nonane- 7-carboxamido)benzyl)- 1-isopropyl-7-methyl- 4,5,6,7-tetrahydro-1H- pyrazolo[4,3-c]pyridine- 3-carboxamide | 660.5 |
| 603 | Ex. 567 | 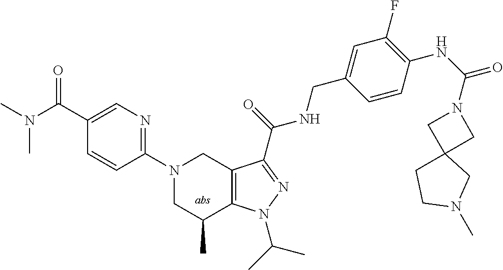 [SMILES: CC(C)n1nc(C(=O)NCc2ccc(NC(=O)N3CC4(CCN(C)C4)C3)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- (6-methyl-2,6- diazaspiro[3.4]octane-2- carboxamido)benzyl)-1- isopropyl-7-methyl- 4,5,6,7-tetrahydro-1H- pyrazolo[4,3-c]pyridine- 3-carboxamide | 646.5 |
| 604 | Ex. 567 | 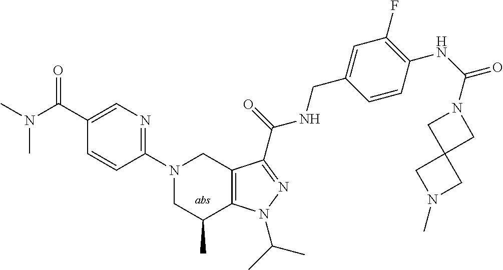 [SMILES: CC(C)n1nc(C(=O)NCc2ccc(NC(=O)N3CC4(CN(C)C4)C3)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- (6-methyl-2,6- diazaspiro[3.3]heptane- 2-carboxamido)benzyl)- 1-isopropyl-7-methyl- 4,5,6,7-tetrahydro-1H- pyrazolo[4,3-c]pyridine- 3-carboxamide | 632.5 |
| 605 | Ex 132^{o} | 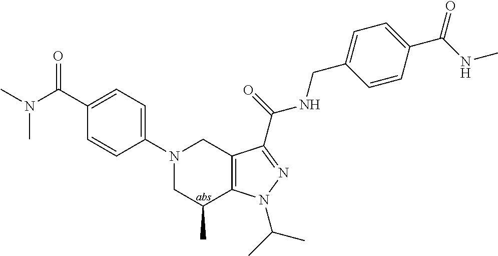 | (S)-5-(4- (dimethylcarbamoyl) phenyl)-1-isopropyl-7- methyl-N-(4- (methylcarbamoyl)benzyl)- 4,5,6,7-tetrahydro-1H- pyrazolo[4,3-c]pyridine- 3-carboxamide | 517.4 |
| 606 | Ex. 567 | 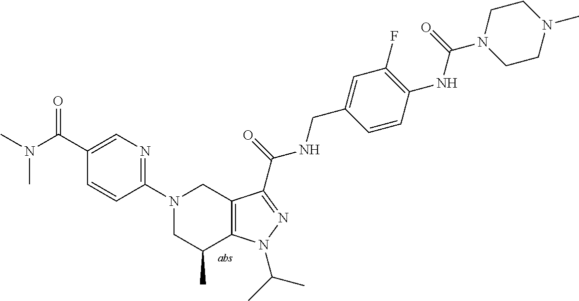 | (S)-5-(5- (dimethylcarbamoyl) pyridin-2-yl)-N-(3-fluoro-4- (4-methylpiperazine-1- carboxamido)benzyl)-1- isopropyl-7-methyl- 4,5,6,7-tetrahydro-1H- pyrazolo[4,3-c]pyridine- 3-carboxamide | 620.5 |
| 607 | Ex. 567 | 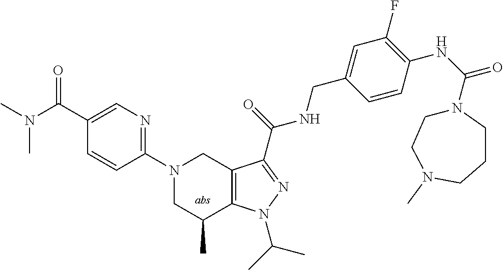 [SMILES: CC(C)n1nc(C(=O)NCc2ccc(NC(=O)N3CCCN(C)CC3)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- (4-methyl-1,4- diazepane-1- carboxamido)benzyl)-1- isopropyl-7-methyl- 4,5,6,7-tetrahydro-1H- pyrazolo[4,3-c]pyridine- 3-carboxamide | 634.5 |
| 608 | Ex. 567 | 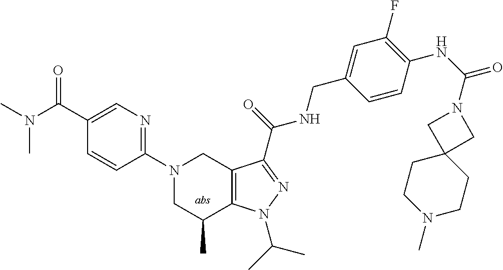 [SMILES: CC(C)n1nc(C(=O)NCc2ccc(NC(=O)N3CC4(CCN(C)CC4)C3)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- (7-methyl-2,7- diazaspiro[3.5]nonane- 2-carboxamido)benzyl)- 1-isopropyl-7-methyl- 4,5,6,7-tetrahydro-1H- pyrazolo[4,3-c]pyridine- 3-carboxamide | 660.6 |
| 609 | Ex. 125 | 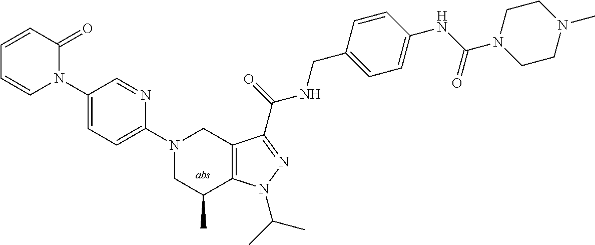 [SMILES: CC(C)n1nc(C(=O)NCc2ccc(NC(=O)N3CCN(C)CC3)cc2)c2c1[C@@H](C)CN(c1ccc(-n3ccccc3=O)cn1)C2] | (S)-1-isopropyl-7- methyl-N-(4-(4- methylpiperazine-1- carboxamido)benzyl)-5- (2-oxo-2H-[1,3′- bipyridin]-6′-yl)-4,5,6,7- tetrahydro-1H- pyrazolo[4,3-c]pyridine- 3-carboxamide | 624.7 |
| 610 | Ex. 125 | 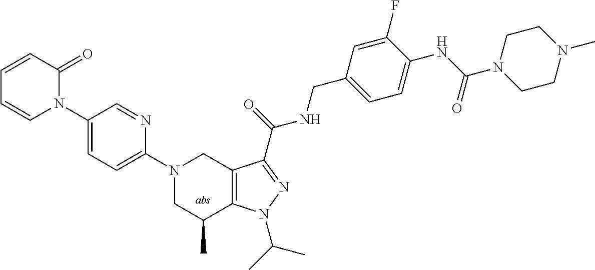 [SMILES: CC(C)n1nc(C(=O)NCc2ccc(NC(=O)N3CCN(C)CC3)c(F)c2)c2c1[C@@H](C)CN(c1ccc(-n3ccccc3=O)cn1)C2] | (S)-N-(3-fluoro-4-(4- methylpiperazine-1- carboxamido)benzyl)-1- isopropyl-7-methyl-5-(2- oxo-2H-[1,3′-bipyridin]- 6′-yl)-4,5,6,7-tetrahydro- 1H-pyrazolo[4,3- c]pyridine-3- carboxamide | 642.7 |
| 611 | Ex. 125 | 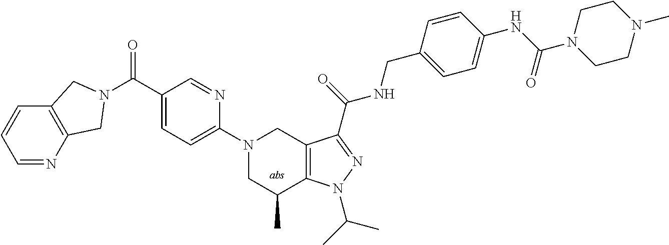 [SMILES: CC(C)n1nc(C(=O)NCc2ccc(NC(=O)N3CCN(C)CC3)cc2)c2c1[C@@H](C)CN(c1ccc(C(=O)N3Cc4cccnc4C3)cn1)C2] | (S)-5-(5-(6,7-dihydro- 5H-pyrrolo[3,4- b]pyridine-6- carbonyl)pyridin-2-yl)-1- isopropyl-7-methyl-N-(4- (4-methylpiperazine-1- carboxamido)benzyl)- 4,5,6,7-tetrahydro-1H- pyrazolo[4,3-c]pyridine- 3-carboxamide | 677.5 |
| 612 | Ex. 125 | 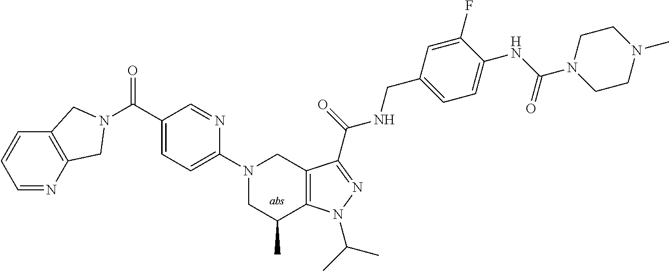 [SMILES: CC(C)n1nc(C(=O)NCc2ccc(NC(=O)N3CCN(C)CC3)c(F)c2)c2c1[C@@H](C)CN(c1ccc(C(=O)N3Cc4cccnc4C3)cn1)C2] | (S)-5-(5-(6,7-dihydro- 5H-pyrrolo[3,4- b]pyridine-6- carbonyl)pyridin-2-yl)-N- (3-fluoro-4-(4- methylpiperazine-1- carboxamido)benzyl)-1- isopropyl-7-methyl- 4,5,6,7-tetrahydro-1H- pyrazolo[4,3-c]pyridine- 3-carboxamide | 695.5 |
| 613 | Ex. 127 | 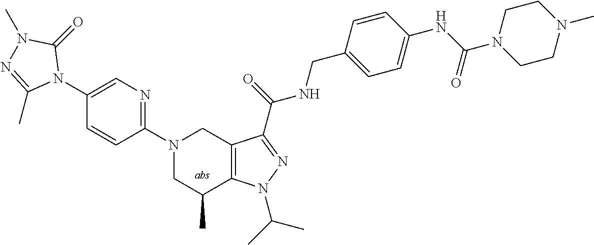 [SMILES: Cc1nn(C)c(=O)n1-c1ccc(N2Cc3c(C(=O)NCc4ccc(NC(=O)N5CCN(C)CC5)cc4)nn(C(C)C)c3[C@@H](C)C2)nc1] | (S)-5-(5-(1,3-dimethyl-5- oxo-1,5-dihydro-4H- 1,2,4-triazol-4-yl)pyridin- 2-yl)-1-isopropyl-7- methyl-N-(4-(4- methylpiperazine-1- carboxamido)benzyl)- 4,5,6,7-tetrahydro-1H- pyrazolo[4,3-c]pyridine- 3-carboxamide | 642.5 |
| 614 | Ex. 125^{q} | 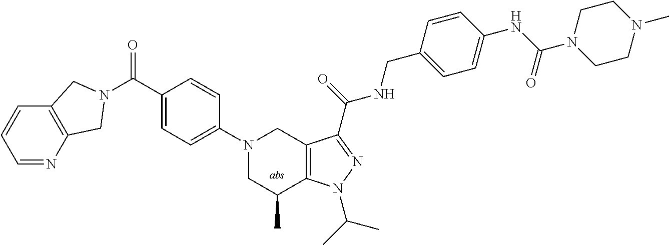 [SMILES: CC(C)n1nc(C(=O)NCc2ccc(NC(=O)N3CCN(C)CC3)cc2)c2c1[C@@H](C)CN(c1ccc(C(=O)N3Cc4cccnc4C3)cc1)C2] | (S)-5-(4-(6,7-dihydro- 5H-pyrrolo[3,4- b]pyridine-6- carbonyl)phenyl)-1- isopropyl-7-methyl-N-(4- (4-methylpiperazine-1- carboxamido)benzyl)- 4,5,6,7-tetrahydro-1H- pyrazolo[4,3-c]pyridine- 3-carboxamide | 676.6 |
| 615 | Ex. 567 | 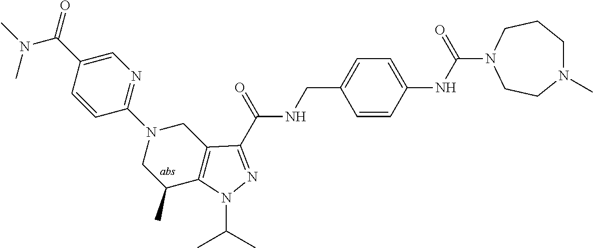 | (S)-5-(5- (dimethylcarbamoyl) pyridin-2-yl)-1-isopropyl-7- methyl-N-(4-(4-methyl- 1,4-diazepane-1- carboxamido)benzyl)- 4,5,6,7-tetrahydro-1H- pyrazolo[4,3-c]pyridine- 3-carboxamide | 616.5 |
| 616 | Ex. 567 | 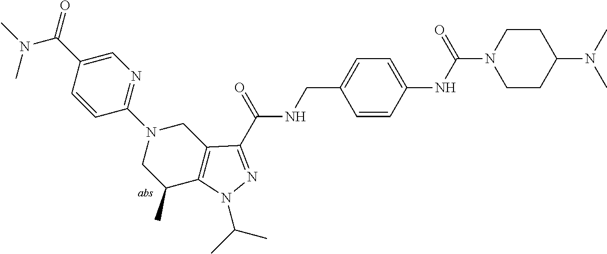 | (S)-N-(4-(4- (dimethylamino)piperidine- 1- carboxamido)benzyl)-5- (5- (dimethylcarbamoyl) pyridin-2-yl)-1-isopropyl-7- methyl-4,5,6,7- tetrahydro-1H- pyrazolo[4,3-c]pyridine- 3-carboxamide | 630.5 |
| 617 | Ex. 567 | 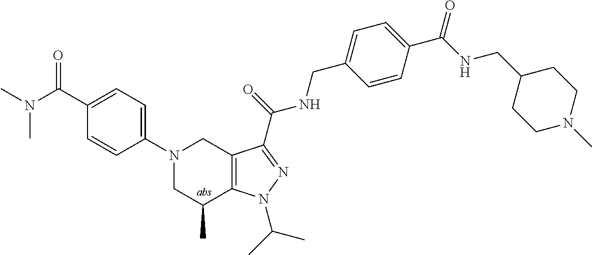 | (S)-5-(4- (dimethylcarbamoyl) phenyl)-1-isopropyl-7- methyl-N-(4-(((1- methylpiperidin-4- yl)methyl)carbamoyl) benzyl)-4,5,6,7-tetrahydro- 1H-pyrazolo[4,3- c]pyridine-3- carboxamide | 614.4 |
| 618 | Ex. 128 | 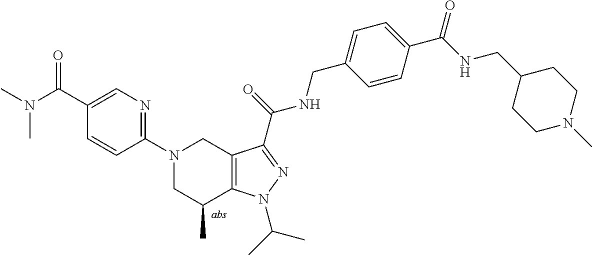 | (S)-5-(5- (dimethylcarbamoyl) pyridin-2-yl)-1-isopropyl-7- methyl-N-(4-(((1- methylpiperidin-4- yl)methyl)carbamoyl) benzyl)-4,5,6,7-tetrahydro- 1H-pyrazolo[4,3- c]pyridine-3- carboxamide | 615.4 |
| 619 | Ex. 485 | 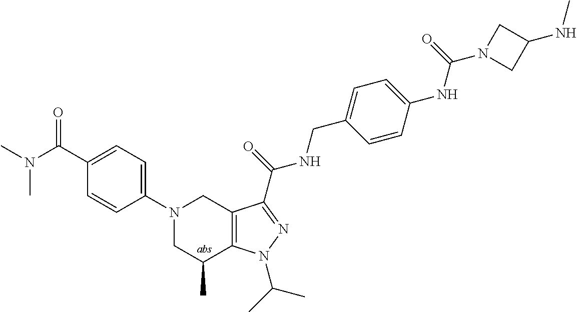 | (S)-5-(4- (dimethylcarbamoyl) phenyl)-1-isopropyl-7- methyl-N-(4-(3- (methylamino)azetidine- 1-carboxamido)benzyl)- 4,5,6,7-tetrahydro-1H- pyrazolo[4,3-c]pyridine- 3-carboxamide | 587.4 |
| 620 | Ex. 485 | 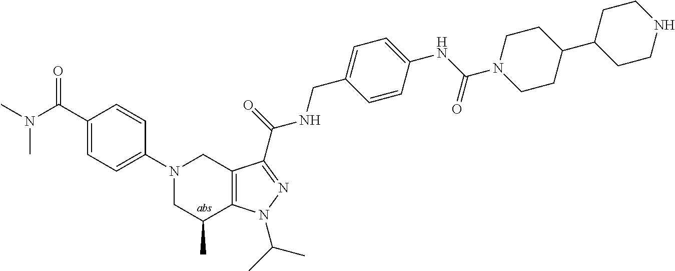 [SMILES: CC(C)n1nc(C(=O)NCc2ccc(NC(=O)N3CCC(C4CCNCC4)CC3)cc2)c2c1[C@@H](C)CN(c1ccc(C(=O)N(C)C)cc1)C2] | (S)-N-(4-([4,4′- bipiperidine]-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 669.6 |
| 621 | Ex. 485 | 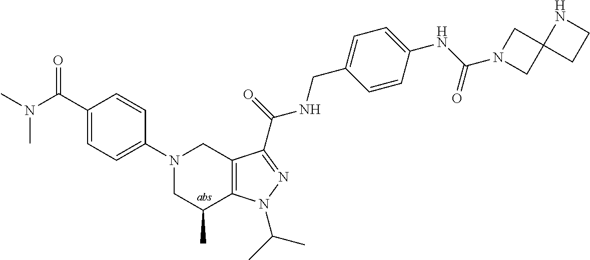 [SMILES: CC(C)n1nc(C(=O)NCc2ccc(NC(=O)N3CC4(CCN4)C3)cc2)c2c1[C@@H](C)CN(c1ccc(C(=O)N(C)C)cc1)C2] | (S)-N-(4-(1,6- diazaspiro[3.3]heptane- 6-carboxamido)benzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 599.5 |
| 622 | Ex. 485 | 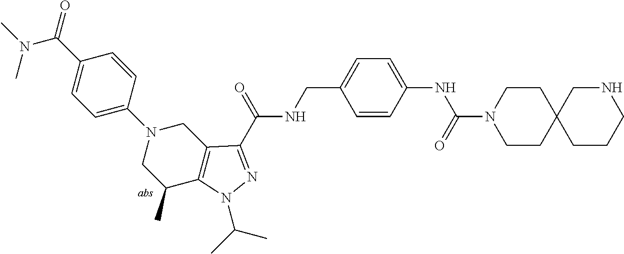 [SMILES: CC(C)n1nc(C(=O)NCc2ccc(NC(=O)N3CCC4(CCCNC4)CC3)cc2)c2c1[C@@H](C)CN(c1ccc(C(=O)N(C)C)cc1)C2] | (S)-N-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)-2,9- diazaspiro[5.5]undecane- 9-carboxamide | 655.6 |
| 623 | Ex. 485 | 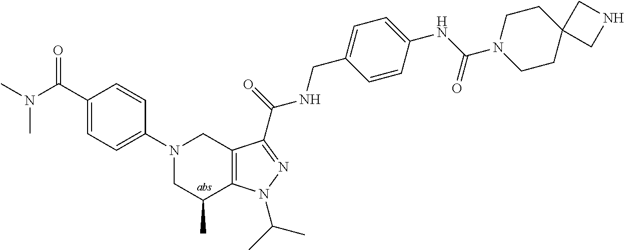 [SMILES: CC(C)n1nc(C(=O)NCc2ccc(NC(=O)N3CCC4(CC3)CNC4)cc2)c2c1[C@@H](C)CN(c1ccc(C(=O)N(C)C)cc1)C2] | (S)-N-(4-(2,7- diazaspiro[3.5]nonane- 7-carboxamido)benzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 627.6 |
| 624 | Ex. 485 | 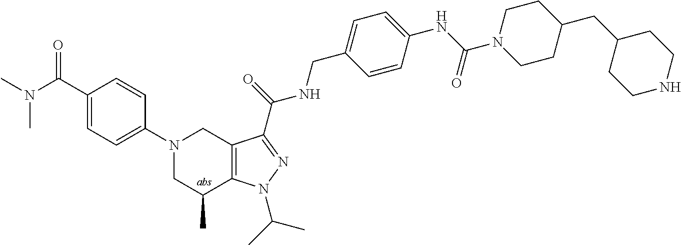 [SMILES: CC(C)n1nc(C(=O)NCc2ccc(NC(=O)N3CCC(CC4CCNCC4)CC3)cc2)c2c1[C@@H](C)CN(c1ccc(C(=O)N(C)C)cc1)C2] | (S)-5-(4- (dimethylcarbamoyl) phenyl)-1-isopropyl-7- methyl-N-(4-(4- (piperidin-4- ylmethyl)piperidine-1- carboxamido)benzyl)- 4,5,6,7-tetrahydro-1H- pyrazolo[4,3-c]pyridine- 3-carboxamide | 683.6 |
| 625 | Ex. 485 | 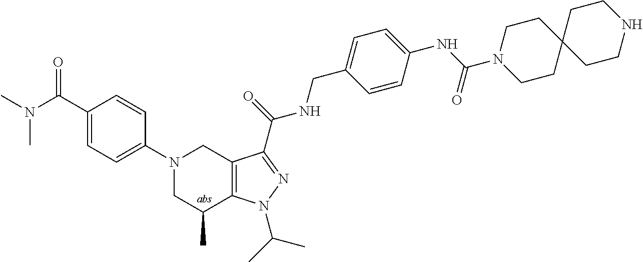 [SMILES: CC(C)n1nc(C(=O)NCc2ccc(NC(=O)N3CCC4(CCNCC4)CC3)cc2)c2c1[C@@H](C)CN(c1ccc(C(=O)N(C)C)cc1)C2] | (S)-N-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)-3,9- diazaspiro[5.5]undecane- 3-carboxamide | 655.6 |
| 626 | Ex. 485 | 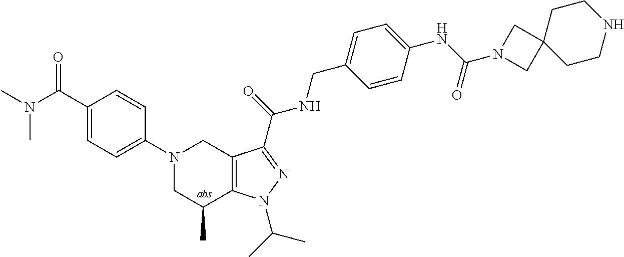 [SMILES: CC(C)n1nc(C(=O)NCc2ccc(NC(=O)N3CC4(CCNCC4)C3)cc2)c2c1[C@@H](C)CN(c1ccc(C(=O)N(C)C)cc1)C2] | (S)-N-(4-(2,7- diazaspiro[3.5]nonane- 2-carboxamido)benzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 627.5 |
| 627 | Ex. 485 | 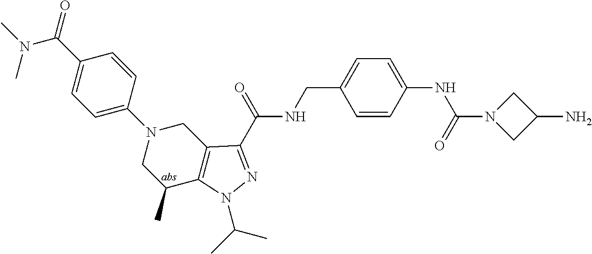 [SMILES: CC(C)n1nc(C(=O)NCc2ccc(NC(=O)N3CC(N)C3)cc2)c2c1[C@@H](C)CN(c1ccc(C(=O)N(C)C)cc1)C2] | (S)-N-(4-(3- aminoazetidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 573.4 |
| 628 | Ex. 485 | 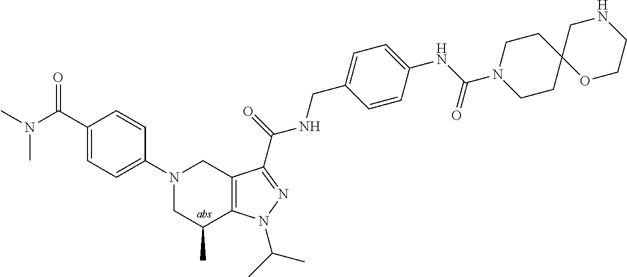 [SMILES: CC(C)n1nc(C(=O)NCc2ccc(NC(=O)N3CCC4(CC3)CNCCO4)cc2)c2c1[C@@H](C)CN(c1ccc(C(=O)N(C)C)cc1)C2] | (S)-N-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)-1-oxa-4,9- diazaspiro[5.5]undecane- 9-carboxamide | 657.6 |
| 629 | Ex. 395^{fo} | 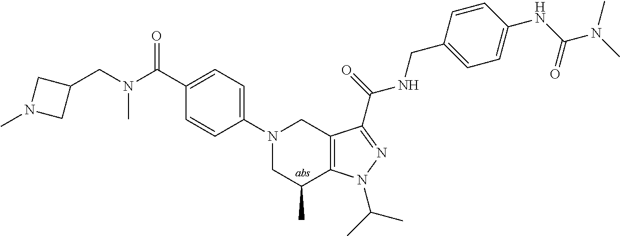 | (S)-N-(4-(3,3- dimethylureido)benzyl)- 1-isopropyl-7-methyl-5- (4-(methyl((1- methylazetidin-3- yl)methyl)carbamoyl) phenyl)-4,5,6,7-tetrahydro- 1H-pyrazolo[4,3- c]pyridine-3- carboxamide | 615.5 |
| 630 | Ex. 485 | 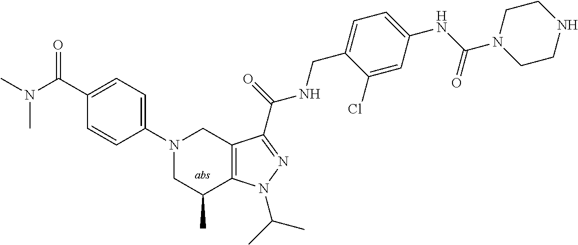 [SMILES: CC(C)n1nc(C(=O)NCc2ccc(NC(=O)N3CCNCC3)cc2Cl)c2c1[C@@H](C)CN(c1ccc(C(=O)N(C)C)cc1)C2] | (S)-N-(2-chloro-4- (piperazine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 621.4 |
| 631 | Ex. 125^{q} | 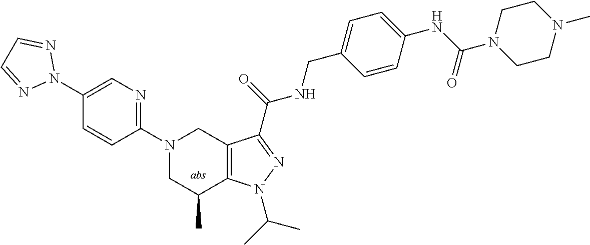 [SMILES: CC(C)n1nc(C(=O)NCc2ccc(NC(=O)N3CCN(C)CC3)cc2)c2c1[C@@H](C)CN(c1ccc(-n3nccn3)cn1)C2] | (S)-5-(5-(2H-1,2,3- triazol-2-yl)pyridin-2-yl)- 1-isopropyl-7-methyl-N- (4-(4-methylpiperazine- 1-carboxamido)benzyl)- 4,5,6,7-tetrahydro-1H- pyrazolo[4,3-c]pyridine- 3-carboxamide | 598.5 |
| 632 | Ex. 567 | 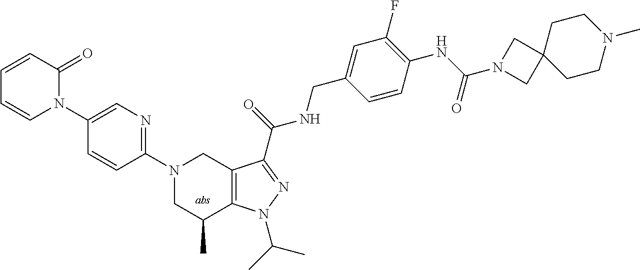 [SMILES: CC(C)n1nc(C(=O)NCc2ccc(NC(=O)N3CC4(CCN(C)CC4)C3)c(F)c2)c2c1[C@@H](C)CN(c1ccc(-n3ccccc3=O)cn1)C2] | (S)-N-(3-fluoro-4-(7- methyl-2,7- diazaspiro[3.5]nonane- 2-carboxamido)benzyl)- 1-isopropyl-7-methyl-5- (2-oxo-2H-[1,3′- bipyridin]-6′-yl)-4,5,6,7- tetrahydro-1H- pyrazolo[4,3-c]pyridine- 3-carboxamide | 682.6 |
| 633 | Ex. 567 | 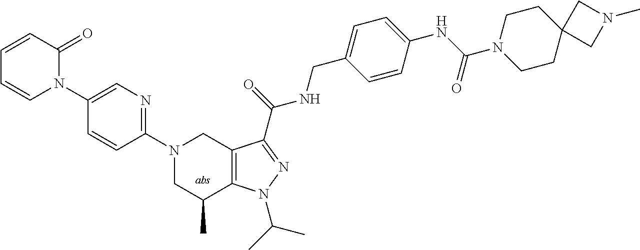 [SMILES: CC(C)n1nc(C(=O)NCc2ccc(NC(=O)N3CCC4(CC3)CN(C)C4)cc2)c2c1[C@@H](C)CN(c1ccc(-n3ccccc3=O)cn1)C2] | (S)-1-isopropyl-7- methyl-N-(4-(2-methyl- 2,7- diazaspiro[3.5]nonane- 7-carboxamido)benzyl)- 5-(2-oxo-2H-[1,3′- bipyridin]-6′-yl)-4,5,6,7- tetrahydro-1H- pyrazolo[4,3-c]pyridine- 3-carboxamide | 664.5 |
| 634 | Ex. 567 | 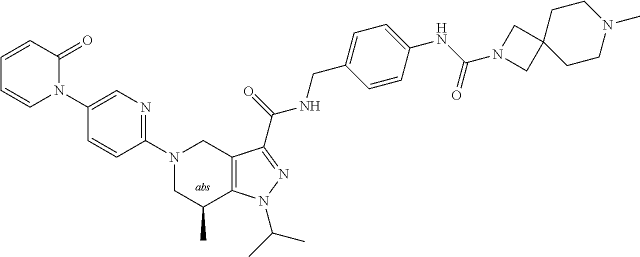 [SMILES: CC(C)n1nc(C(=O)NCc2ccc(NC(=O)N3CC4(CCN(C)CC4)C3)cc2)c2c1[C@@H](C)CN(c1ccc(-n3ccccc3=O)cn1)C2] | (S)-1-isopropyl-7- methyl-N-(4-(7-methyl- 2,7- diazaspiro[3.5]nonane- 2-carboxamido)benzyl)- 5-(2-oxo-2H-[1,3′- bipyridin]-6′-yl)-4,5,6,7- tetrahydro-1H- pyrazolo[4,3-c]pyridine- 3-carboxamide | 664.6 |
| 635 | Ex. 395^{fo} | 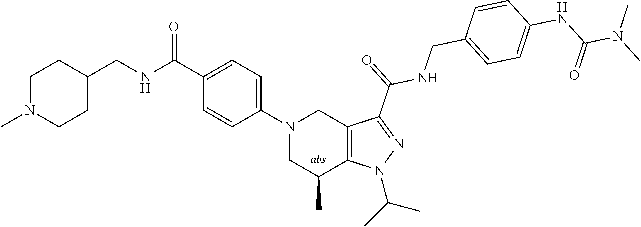 | (S)-N-(4-(3,3- dimethylureido)benzyl)- 1-isopropyl-7-methyl-5- (4-(methyl((1- methylpiperidin-4- yl)methyl)carbamoyl) phenyl)-4,5,6,7-tetrahydro- 1H-pyrazolo[4,3- c]pyridine-3- carboxamide | 643.5 |
| 636 | Ex. 395^{fo} | 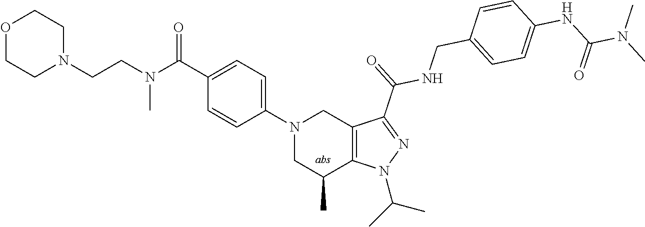 [SMILES: CC(C)n1nc(C(=O)NCc2ccc(NC(=O)N(C)C)cc2)c2c1[C@@H](C)CN(c1ccc(C(=O)N(C)CCN3CCOCC3)cc1)C2] | (S)-N-(4-(3,3- dimethylureido)benzyl)- 1-isopropyl-7-methyl-5- (4-(methyl(2- morpholinoethyl)carbamoyl) phenyl)-4,5,6,7- tetrahydro-1H- pyrazolo[4,3-c]pyridine- 3-carboxamide | 645.5 |
| 637 | Ex. 395^{fop} | 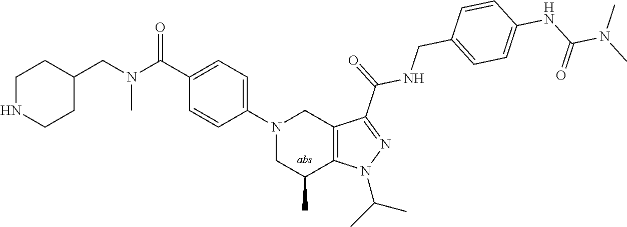 [SMILES: CC(C)n1nc(C(=O)NCc2ccc(NC(=O)N(C)C)cc2)c2c1[C@@H](C)CN(c1ccc(C(=O)N(C)CC3CCNCC3)cc1)C2] | (S)-N-(4-(3,3- dimethylureido)benzyl)- 1-isopropyl-7-methyl-5- (4-(methyl(piperidin-4- ylmethyl)carbamoyl) phenyl)-4,5,6,7-tetrahydro- 1H-pyrazolo[4,3- c]pyridine-3- carboxamide | 629.7 |
| 638 | Ex. 567 | 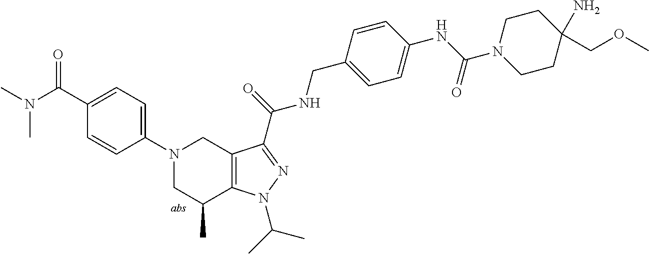 | (S)-N-(4-(4-amino-4- (methoxymethyl) piperidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 645.5 |
| 639 | Ex. 567 | 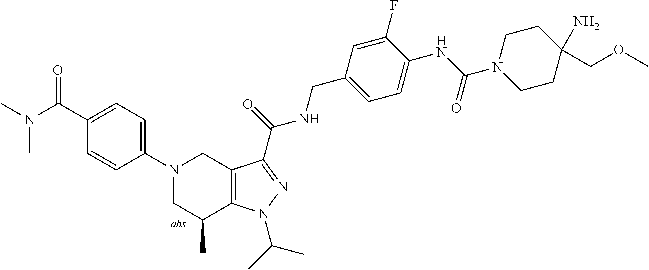 | (S)-N-(4-(4-amino-4- (methoxymethyl)piperidine- 1-carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 663.6 |
| 640 | Ex. 567 | 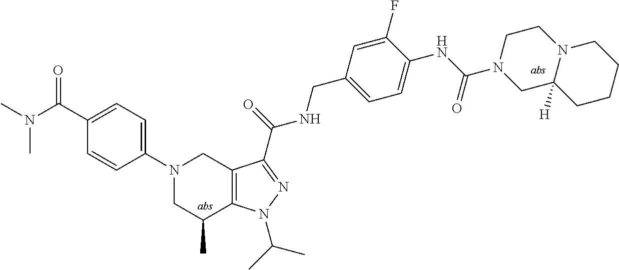 [SMILES: CC(C)n1nc(C(=O)NCc2ccc(NC(=O)N3CCN4CCCC[C@H]4C3)c(F)c2)c2c1[C@@H](C)CN(c1ccc(C(=O)N(C)C)cc1)C2] | (S)-N-(4-(((S)-5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2- fluorophenyl)octahydro- 2H-pyrido[1,2- a]pyrazine-2- carboxamide | 659.6 |
| 641 | Ex. 567 | 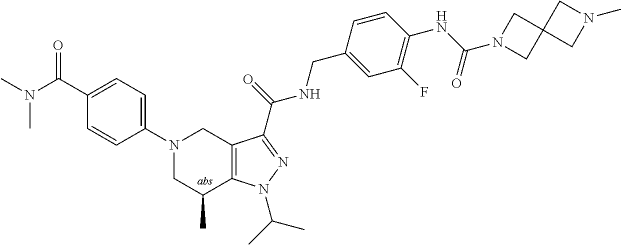 | (S)-5-(4- (dimethylcarbamoyl) phenyl)-N-(3-fluoro-4-(6- methyl-2,6- diazaspiro[3.3]heptane- 2-carboxamido)benzyl)- 1-isopropyl-7-methyl- 4,5,6,7-tetrahydro-1H- pyrazolo[4,3-c]pyridine- 3-carboxamide | 631.5 |
| 642 | Ex. 567 | 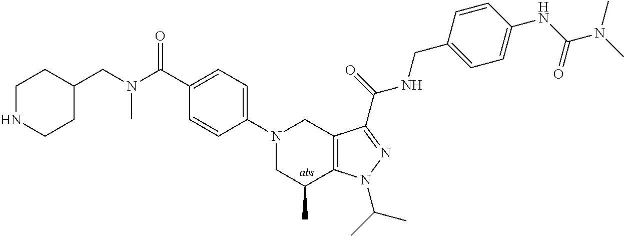 [SMILES: CC(C)n1nc(C(=O)NCc2ccc(NC(=O)N(C)C)cc2)c2c1[C@@H](C)CN(c1ccc(C(=O)N(C)CC3CCNCC3)cc1)C2] | (S)-N-(4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2-fluorophenyl)-2- methyl-2,8- diazaspiro[4.5]decane- 8-carboxamide | 673.6 |
| 643 | Ex. 567 | 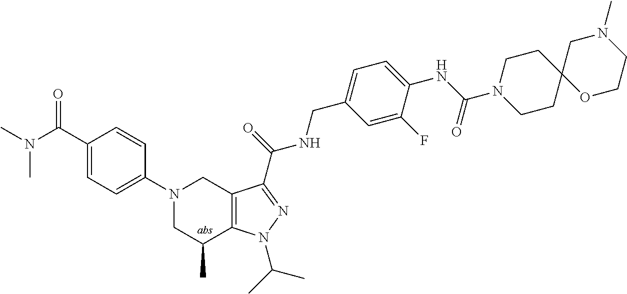 [SMILES: CC(C)n1nc(C(=O)NCc2ccc(NC(=O)N3CCC4(CC3)CN(C)CCO4)c(F)c2)c2c1[C@@H](C)CN(c1ccc(C(=O)N(C)C)cc1)C2] | (S)-N-(4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2-fluorophenyl)-4- methyl-1-oxa-4,9- diazaspiro[5.5]undecane- 9-carboxamide | 689.7 |
| 644 | Ex. 567 | 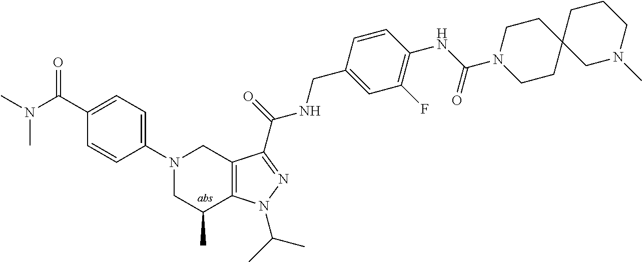 | (S)-N-(4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2-fluorophenyl)-2- methyl-2,9- diazaspiro[5.5]undecane- 9-carboxamide | 687.6 |
| 645 | Ex. 567 | 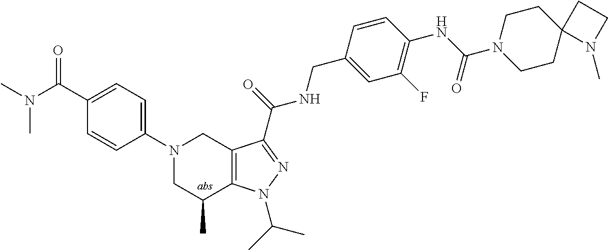 [SMILES: CC(C)n1nc(C(=O)NCc2ccc(NC(=O)N3CCC4(CC3)CCN4C)c(F)c2)c2c1[C@@H](C)CN(c1ccc(C(=O)N(C)C)cc1)C2] | (S)-5-(4- (dimethylcarbamoyl)phenyl)- N-(3-fluoro-4-(1- methyl-1,7- diazaspiro[3.5]nonane- 7-carboxamido)benzyl)- 1-isopropyl-7-methyl- 4,5,6,7-tetrahydro-1H- pyrazolo[4,3-c]pyridine- 3-carboxamide | 659.6 |
| 646 | Ex. 567 | 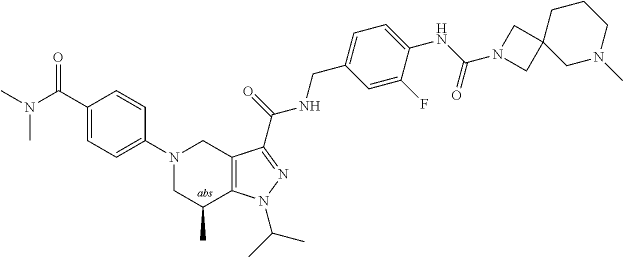 | (S)-5-(4- (dimethylcarbamoyl) phenyl)-N-(3-fluoro-4-(6- methyl-2,6- diazaspiro[3.5]nonane- 2-carboxamido)benzyl)- 1-isopropyl-7-methyl- 4,5,6,7-tetrahydro-1H- pyrazolo[4,3-c]pyridine- 3-carboxamide | 659.6 |
| 647 | Ex. 567 | 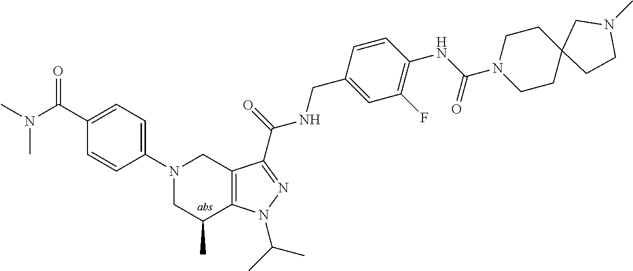 | (S)-5-(4- (dimethylcarbamoyl) phenyl)-N-(3-fluoro-4-(2- methyl-2,7- diazaspiro[3.5]nonane- 7-carboxamido)benzyl)- 1-isopropyl-7-methyl- 4,5,6,7-tetrahydro-1H- pyrazolo[4,3-c]pyridine- 3-carboxamide | 659.5 |
| 648 | Ex. 567 | 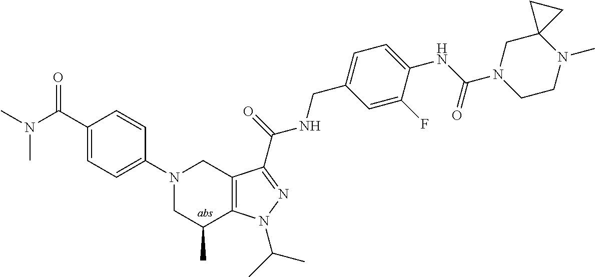 | (S)-5-(4- (dimethylcarbamoyl) phenyl)-N-(3-fluoro-4-(4- methyl-4,7- diazaspiro[2.5]octane-7- carboxamido)benzyl)-1- isopropyl-7-methyl- 4,5,6,7-tetrahydro-1H- pyrazolo[4,3-c]pyridine- 3-carboxamide | 645.6 |
| 649 | Ex. 515^{y} | 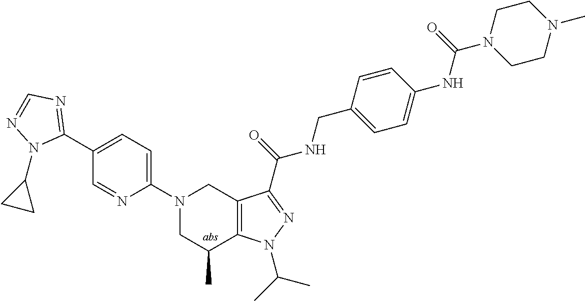 [SMILES: CC(C)n1nc(C(=O)NCc2ccc(NC(=O)N3CCN(C)CC3)cc2)c2c1[C@@H](C)CN(c1ccc(-c3ncnn3C3CC3)cn1)C2] | (S)-5-(5-(1-cyclopropyl- 1H-1,2,4-triazol-5- yl)pyridin-2-yl)-1- isopropyl-7-methyl-N-(4- (4-methylpiperazine-1- carboxamido)benzyl)- 4,5,6,7-tetrahydro-1H- pyrazolo[4,3-c]pyridine- 3-carboxamide | 638.5 |
| 650 | Ex. 567 | 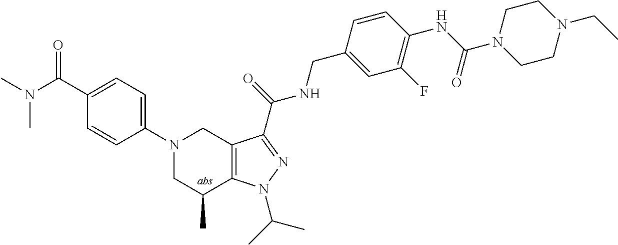 [SMILES: CCN1CCN(C(=O)Nc2ccc(CNC(=O)c3nn(C(C)C)c4c3CN(c3ccc(C(=O)N(C)C)cc3)C[C@@H]4C)cc2F)CC1] | (S)-5-(4- (dimethylcarbamoyl) phenyl)-N-(4-(4- ethylpiperazine-1- carboxamido)-3- fluorobenzyl)-1- isopropyl-7-methyl- 4,5,6,7-tetrahydro-1H- pyrazolo[4,3-c]pyridine- 3-carboxamide | 633.5 |
| 651 | Ex. 127ⁱ | 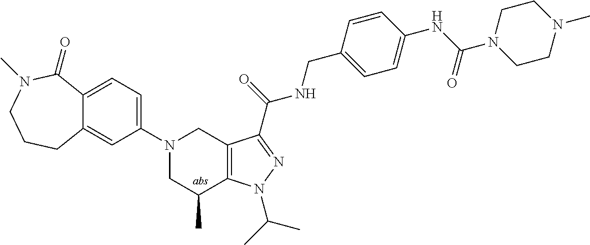 | (S)-1-isopropyl-7- methyl-5-(2-methyl-1- oxo-2,3,4,5-tetrahydro- 1H-benzo[c]azepin-7-yl)- N-(4-(4- methylpiperazine-1- carboxamido)benzyl)- 4,5,6,7-tetrahydro-1H- pyrazolo[4,3-c]pyridine- 3-carboxamide | 627.5 |
| 652 | Ex. 127ⁱ | 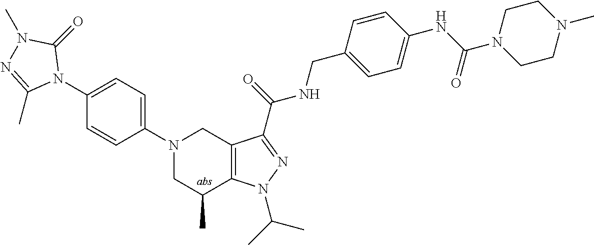 [SMILES: Cc1nn(C)c(=O)n1-c1ccc(N2Cc3c(C(=O)NCc4ccc(NC(=O)N5CCN(C)CC5)cc4)nn(C(C)C)c3[C@@H](C)C2)cc1] | (S)-5-(4-(1,3-dimethyl-5- oxo-1,5-dihydro-4H- 1,2,4-triazol-4- yl)phenyl)-1-isopropyl-7- methyl-N-(4-(4- methylpiperazine-1- carboxamido)benzyl)- 4,5,6,7-tetrahydro-1H- pyrazolo[4,3-c]pyridine- 3-carboxamide | 641.3 |
| 653 | Ex. 125^{wx} | 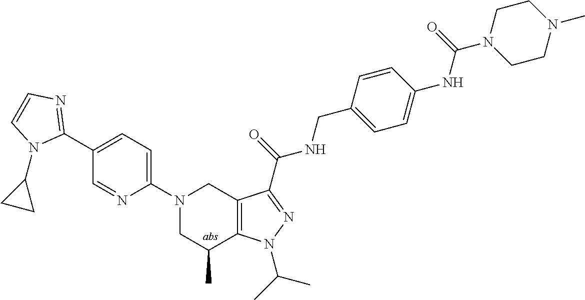 [SMILES: CC(C)n1nc(C(=O)NCc2ccc(NC(=O)N3CCN(C)CC3)cc2)c2c1[C@@H](C)CN(c1ccc(-c3nccn3C3CC3)cn1)C2] | (S)-5-(5-(1-cyclopropyl- 1H-imidazol-2-yl)pyridin- 2-yl)-1-isopropyl-7- methyl-N-(4-(4- methylpiperazine-1- carboxamido)benzyl)- 4,5,6,7-tetrahydro-1H- pyrazolo[4,3-c]pyridine- 3-carboxamide | 637.5 |
| 654 | Ex. 125^{wx} | 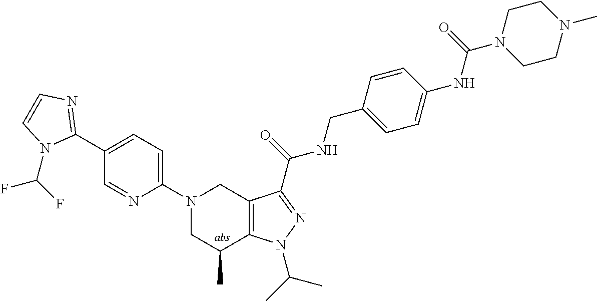 [SMILES: CC(C)n1nc(C(=O)NCc2ccc(NC(=O)N3CCN(C)CC3)cc2)c2c1[C@@H](C)CN(c1ccc(-c3nccn3C(F)F)cn1)C2] | (S)-5-(5-(1- (difluoromethyl)-1H- imidazol-2-yl)pyridin-2- yl)-1-isopropyl-7-methyl- N-(4-(4- methylpiperazine-1- carboxamido)benzyl)- 4,5,6,7-tetrahydro-1H- pyrazolo[4,3-c]pyridine- 3-carboxamide | 647.5 |
| 655 | Ex. 125^{wx} | 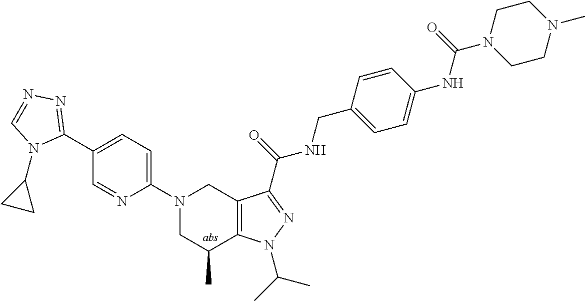 [SMILES: CC(C)n1nc(C(=O)NCc2ccc(NC(=O)N3CCN(C)CC3)cc2)c2c1[C@@H](C)CN(c1ccc(-c3nncn3C3CC3)cn1)C2] | (S)-5-(5-(4-cyclopropyl- 4H-1,2,4-triazol-3- yl)pyridin-2-yl)-1- isopropyl-7-methyl-N-(4- (4-methylpiperazine-1- carboxamido)benzyl)- 4,5,6,7-tetrahydro-1H- pyrazolo[4,3-c]pyridine- 3-carboxamide | 638.6 |
| 656 | Ex. 127ⁱ | 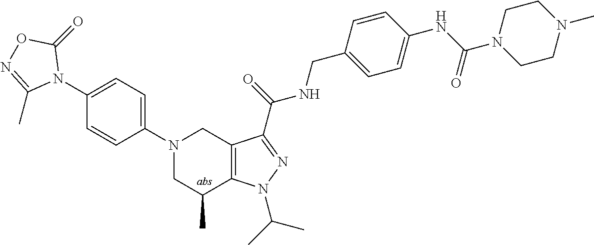 [SMILES: Cc1noc(=O)n1-c1ccc(N2Cc3c(C(=O)NCc4ccc(NC(=O)N5CCN(C)CC5)cc4)nn(C(C)C)c3[C@@H](C)C2)cc1] | (S)-1-isopropyl-7- methyl-5-(4-(3-methyl-5- oxo-1,2,4-oxadiazol- 4(5H)-yl)phenyl)-N-(4- (4-methylpiperazine-1- carboxamido)benzyl)- 4,5,6,7-tetrahydro-1H- pyrazolo[4,3-c]pyridine- 3-carboxamide | 628.5 |
| 657 | Ex. 127ⁱ | 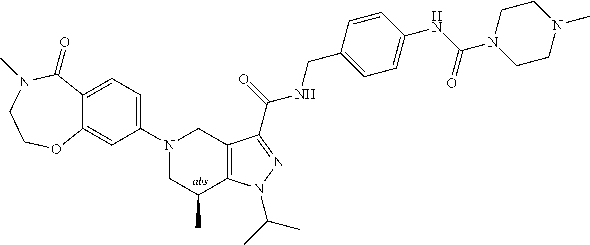 | (S)-1-isopropyl-7- methyl-5-(4-methyl-5- oxo-2,3,4,5- tetrahydrobenzo[f][1,4] oxazepin-8-yl)-N-(4-(4- methylpiperazine-1- carboxamido)benzyl)- 4,5,6,7-tetrahydro-1H- pyrazolo[4,3-c]pyridine- 3-carboxamide | 629.4 |
| 658 | Ex. 515^{y} | 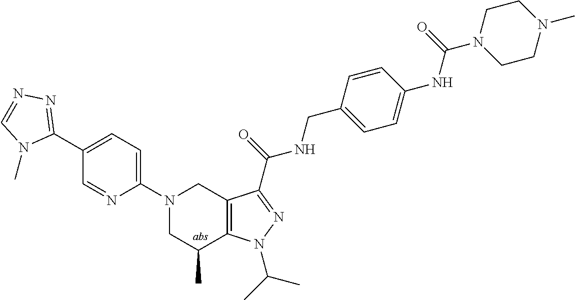 [SMILES: CC(C)n1nc(C(=O)NCc2ccc(NC(=O)N3CCN(C)CC3)cc2)c2c1[C@@H](C)CN(c1ccc(-c3nncn3C)cn1)C2] | (S)-1-isopropyl-7- methyl-5-(5-(4-methyl- 4H-1,2,4-triazol-3- yl)pyridin-2-yl)-N-(4-(4- methylpiperazine-1- carboxamido)benzyl)- 4,5,6,7-tetrahydro-1H- pyrazolo[4,3-c]pyridine- 3-carboxamide | 612.5 |
| 659 | Ex. 567 | 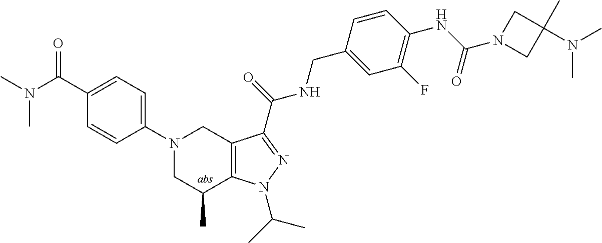 | (S)-N-(4-(3- (dimethylamino)-3- methylazetidine-1- carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 633.3 |
| 660 | Ex. 567 | 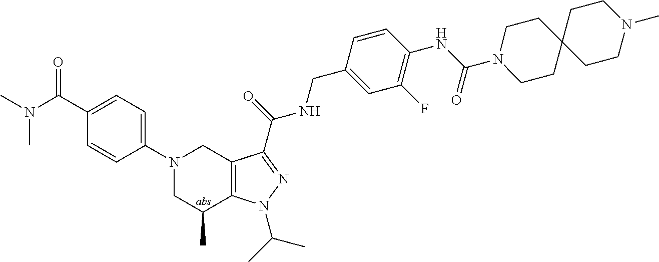 | (S)-N-(4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2-fluorophenyl)-9- methyl-3,9- diazaspiro[5.5]undecane- 3-carboxamide | 687.4 |
| 661 | Ex. 395^{fo} | 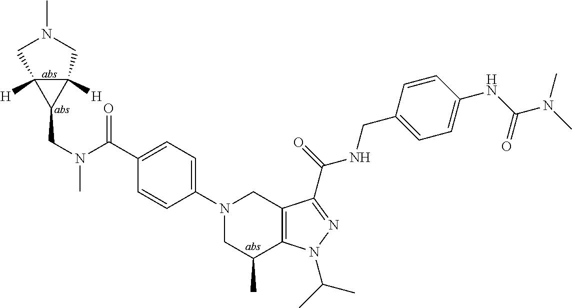 [SMILES: CC(C)n1nc(C(=O)NCc2ccc(NC(=O)N(C)C)cc2)c2c1[C@@H](C)CN(c1ccc(C(=O)N(C)C[C@H]3[C@@H]4CN(C)C[C@@H]43)cc1)C2] | (S)-N-(4-(3,3- dimethylureido)benzyl)- 1-isopropyl-7-methyl-5- (4-(methyl(((1R,5S,6r)- 3-methyl-3- azabicyclo[3.1.0]hexan- 6- yl)methyl)carbamoyl) phenyl)-4,5,6,7-tetrahydro- 1H-pyrazolo[4,3- c]pyridine-3- carboxamide | 641.5 |
| 662 | Ex. 395^{fo} | 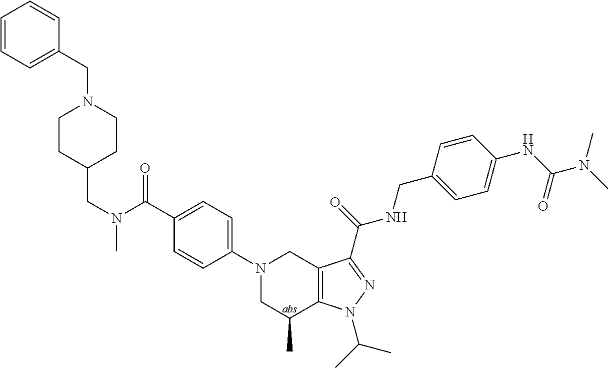 [SMILES: CC(C)n1nc(C(=O)NCc2ccc(NC(=O)N(C)C)cc2)c2c1[C@@H](C)CN(c1ccc(C(=O)N(C)CC3CCN(Cc4ccccc4)CC3)cc1)C2] | (S)-5-(4-(((1- benzylpiperidin-4- yl)methyl)(methyl) carbamoyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 720.3 |
| 663 | Ex. 125^{q} | 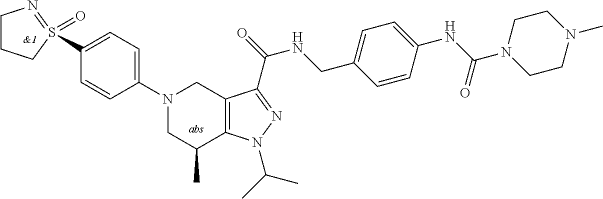 [SMILES: CC(C)n1nc(C(=O)NCc2ccc(NC(=O)N3CCN(C)CC3)cc2)c2c1[C@@H](C)CN(c1ccc([S@]3(=O)=NCCC3)cc1)C2] | (S)-1-isopropyl-7- methyl-N-(4-(4- methylpiperazine-1- carboxamido)benzyl)-5- (4-((S)-1-oxido-4,5- dihydro-3H-1l6- isothiazol-1-yl)phenyl)- 4,5,6,7-tetrahydro-1H- pyrazolo[4,3-c]pyridine- 3-carboxamide | 633.3 |
| 664 | Ex. 132^{or} | 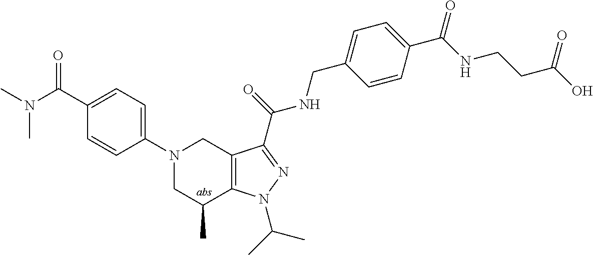 [SMILES: CC(C)n1nc(C(=O)NCc2ccc(C(=O)NCCC(=O)O)cc2)c2c1[C@@H](C)CN(c1ccc(C(=O)N(C)C)cc1)C2] | (S)-3-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) benzamido)propanoic acid | 575.4 |
| 665 | Ex. 567 | 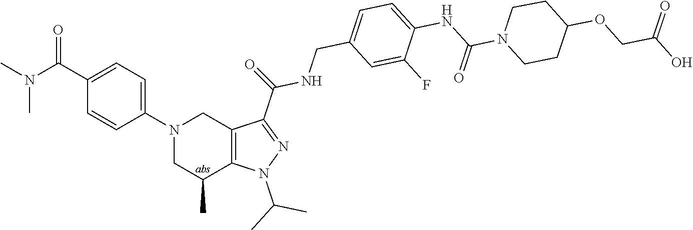 [SMILES: CC(C)n1nc(C(=O)NCc2ccc(NC(=O)N3CCC(OCC(=O)O)CC3)c(F)c2)c2c1[C@@H](C)CN(c1ccc(C(=O)N(C)C)cc1)C2] | (S)-2-((1-((4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2- fluorophenyl)carbamoyl) piperidin-4-yl)oxy)acetic acid | 678.5 |
| 666 | Ex. 127ⁱ | 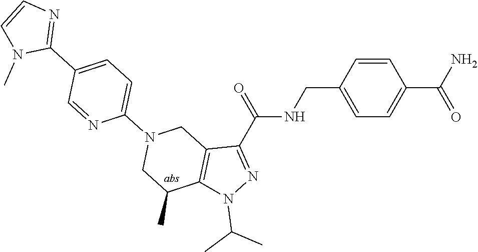 [SMILES: CC(C)n1nc(C(=O)NCc2ccc(C(N)=O)cc2)c2c1[C@@H](C)CN(c1ccc(-c3nccn3C)cn1)C2] | (S)-N-(4- carbamoylbenzyl)-1- isopropyl-7-methyl-5-(5- (1-methyl-1H-imidazol- 2-yl)pyridin-2-yl)-4,5,6,7- tetrahydro-1H- pyrazolo[4,3-c]pyridine- 3-carboxamide | 513.3 |
| 667^{∧} | Ex. 395^{fo} | 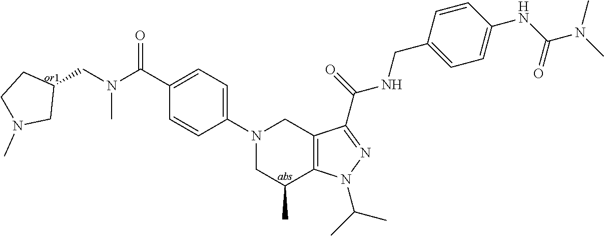 [SMILES: CC(C)n1nc(C(=O)NCc2ccc(NC(=O)N(C)C)cc2)c2c1[C@@H](C)CN(c1ccc(C(=O)N(C)C[C@H]3CCN(C)C3)cc1)C2] | (S)-N-(4-(3,3- dimethylureido)benzyl)- 1-isopropyl-7-methyl-5- (4-(methyl(((R*)-1- methylpyrrolidin-3- yl)methyl)carbamoyl) phenyl)-4,5,6,7-tetrahydro- 1H-pyrazolo[4,3- c]pyridine-3- carboxamide | 629.5 |
| 668^{∧} | Ex. 395^{fo} | 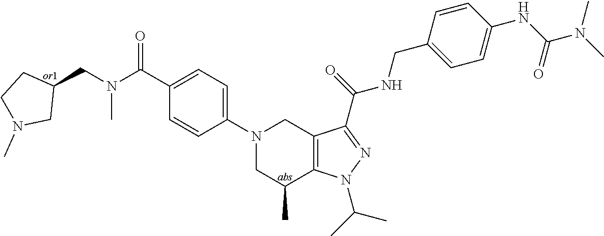 [SMILES: CC(C)n1nc(C(=O)NCc2ccc(NC(=O)N(C)C)cc2)c2c1[C@@H](C)CN(c1ccc(C(=O)N(C)C[C@@H]3CCN(C)C3)cc1)C2] | (S)-N-(4-(3,3- dimethylureido)benzyl)- 1-isopropyl-7-methyl-5- (4-(methyl(((R*)-1- methylpyrrolidin-3- yl)methyl)carbamoyl) phenyl)-4,5,6,7-tetrahydro- 1H-pyrazolo[4,3- c]pyridine-3- carboxamide | 629.5 |
| 669 | Ex. 567 | 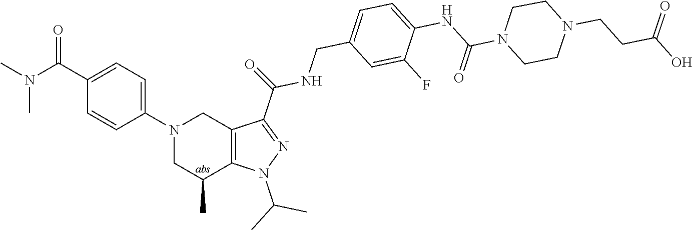 [SMILES: CC(C)n1nc(C(=O)NCc2ccc(NC(=O)N3CCN(CCC(=O)O)CC3)c(F)c2)c2c1[C@@H](C)CN(c1ccc(C(=O)N(C)C)cc1)C2] | (S)-3-(4-((4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2- fluorophenyl)carbamoyl) piperazin-1-yl)propanoic acid | 677.5 |
| 670^{∧} | Ex. 567 | 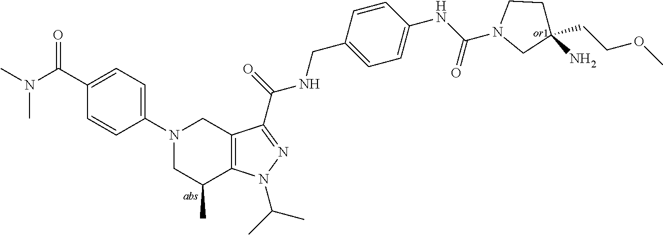 | (S)-N-(4-((R*)-3-amino- 3-(2- methoxyethyl)pyrrolidine- 1-carboxamido)benzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 645.3 |
| 671^{∧} | Ex. 567 | 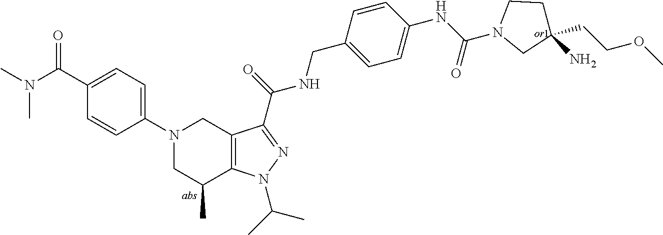 | (S)-N-(4-((R*)-3-amino- 3-(2- methoxyethyl)pyrrolidine- 1-carboxamido)benzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 645.4 |
| 672^{∧} | Ex. 567 | 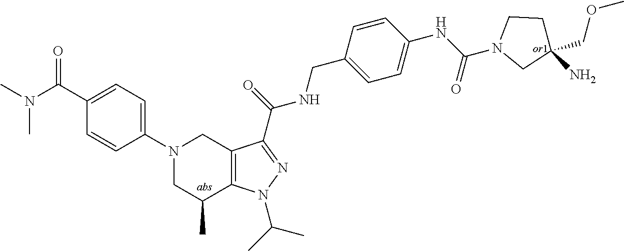 | (S)-N-(4-((R*)-3-amino- 3- (methoxymethyl)pyrrolidine- 1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 631.3 |
| 673^{∧} | Ex. 567 | 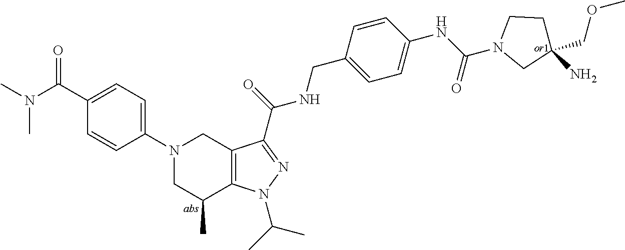 | (S)-N-(4-((R*)-3-amino- 3- (methoxymethyl)pyrrolidine- 1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 631.3 |
| 674 | Ex. 127^{q} | 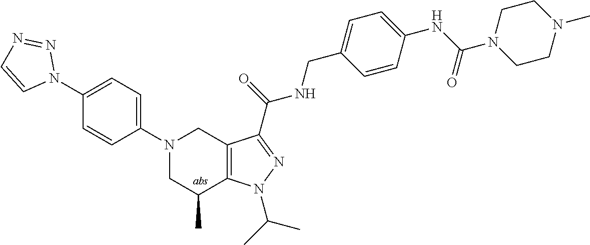 [SMILES: CC(C)n1nc(C(=O)NCc2ccc(NC(=O)N3CCN(C)CC3)cc2)c2c1[C@@H](C)CN(c1ccc(-n3ccnn3)cc1)C2] | (S)-5-(4-(1H-1,2,3- triazol-1-yl)phenyl)-1- isopropyl-7-methyl-N-(4- (4-methylpiperazine-1- carboxamido)benzyl)- 4,5,6,7-tetrahydro-1H- pyrazolo[4,3-c]pyridine- 3-carboxamide | 597.4 |
| 675 | Ex. 395^{fo} | 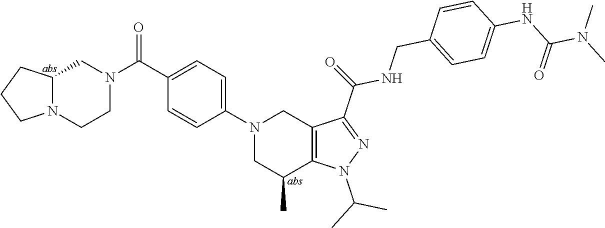 [SMILES: CC(C)n1nc(C(=O)NCc2ccc(NC(=O)N(C)C)cc2)c2c1[C@@H](C)CN(c1ccc(C(=O)N3CCN4CCC[C@@H]4C3)cc1)C2] | (S)-N-(4-(3,3- dimethylureido)benzyl)- 1-isopropyl-7-methyl-5- (4-((R)- octahydropyrrolo[1,2- a]pyrazine-2- carbonyl)phenyl)- 4,5,6,7-tetrahydro-1H- pyrazolo[4,3-c]pyridine- 3-carboxamide | 627.6 |
| 676 | Ex. 193^{fo} | 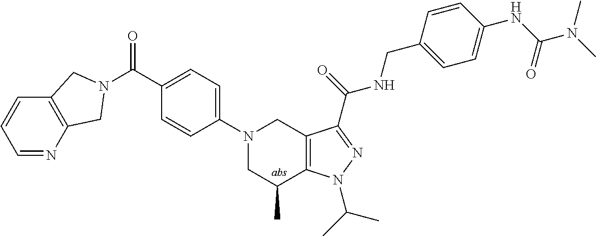 [SMILES: CC(C)n1nc(C(=O)NCc2ccc(NC(=O)N(C)C)cc2)c2c1[C@@H](C)CN(c1ccc(C(=O)N3Cc4cccnc4C3)cc1)C2] | (S)-5-(4-(6,7-dihydro- 5H-pyrrolo[3,4- b]pyridine-6- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 621.5 |
| 677 | | 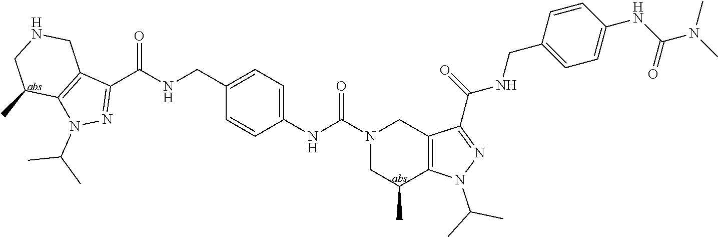 | (S)-N³-(4-(3,3- dimethylureido)benzyl)- 1-isopropyl-N⁵-(4-(((S)- 1-isopropyl-7-methyl- 4,5,6,7-tetrahydro-1H- pyrazolo[4,3-c]pyridine- 3- carboxamido)methyl) phenyl)-7-methyl-1,4,6,7- tetrahydro-5H- pyrazolo[4,3-c]pyridine- 3,5-dicarboxamide | 752.4 |
| 678 | Ex. 567 | 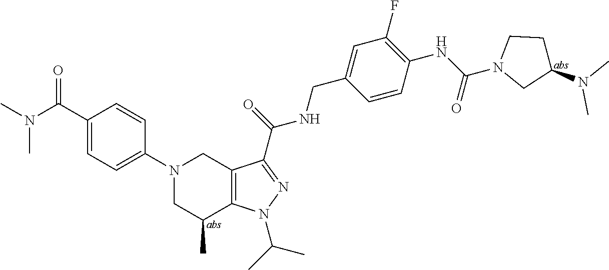 | (S)-N-(4-((R)-3- (dimethylamino)pyrrolidine- 1-carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 633.4 |
| 679 | Ex. 567 | 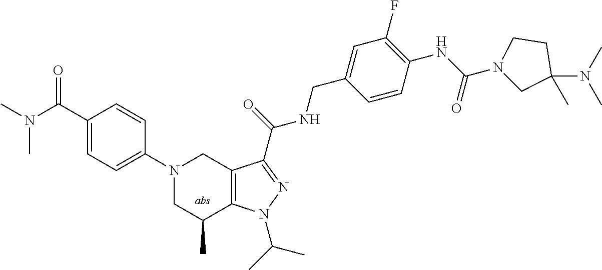 | (7S)-N-(4-(3- (dimethylamino)-3- methylpyrrolidine-1- carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 647.6 |
| 680 | Ex. 567 | 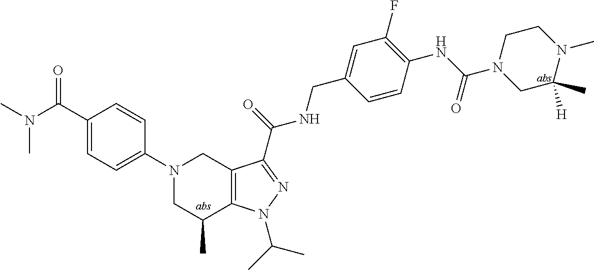 | (S)-5-(4- (dimethylcarbamoyl) phenyl)-N-(4-((S)-3,4- dimethylpiperazine-1- carboxamido)-3- fluorobenzyl)-1- isopropyl-7-methyl- 4,5,6,7-tetrahydro-1H- pyrazolo[4,3-c]pyridine- 3-carboxamide | 633.6 |
| 681 | Ex. 567 | 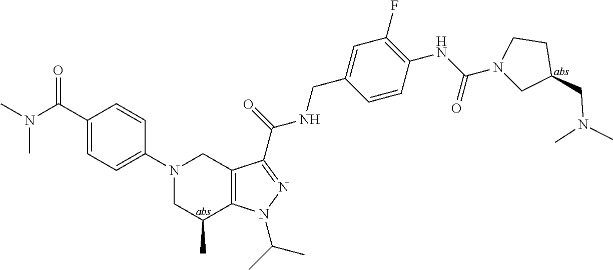 | (S)-N-(4-((S)-3- ((dimethylamino)methyl) pyrrolidine-1- carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 647.6 |
| 682 | Ex. 567 | 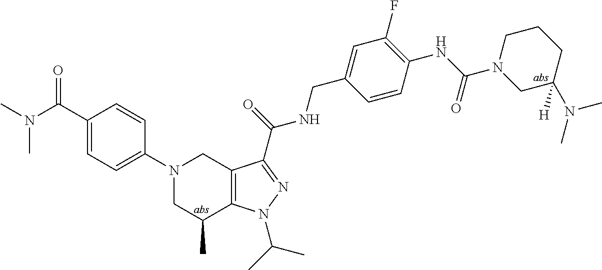 | (S)-N-(4-((R)-3- (dimethylamino)piperidine- 1-carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 647.6 |
| 683 | Ex. 567 | 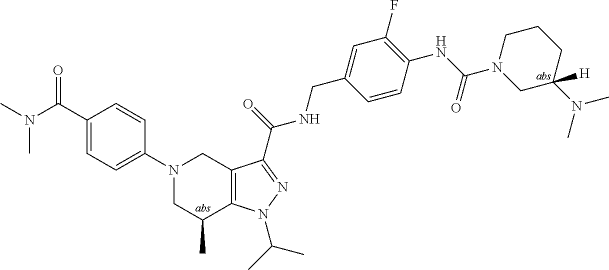 | (S)-N-(4-((S)-3- (dimethylamino)piperidine- 1-carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 647.6 |
| 684 | Ex. 567 | 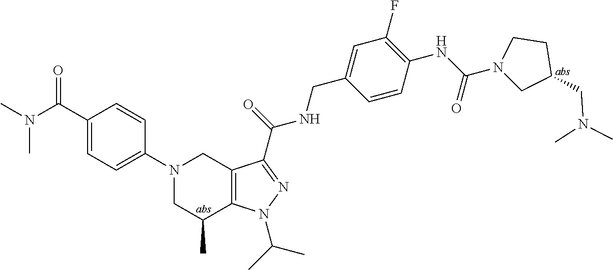 | (S)-N-(4-((R)-3- ((dimethylamino)methyl) pyrrolidine-1- carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 647.6 |
| 685 | Ex. 567 | 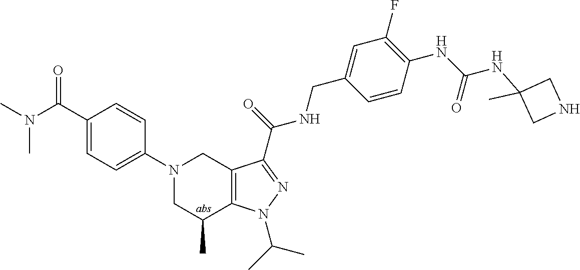 [SMILES: CC(C)n1nc(C(=O)NCc2ccc(NC(=O)NC3(C)CNC3)c(F)c2)c2c1[C@@H](C)CN(c1ccc(C(=O)N(C)C)cc1)C2] | (S)-5-(4- (dimethylcarbamoyl) phenyl)-N-(3-fluoro-4-(3-(3- methylazetidin-3- yl)ureido)benzyl)-1- isopropyl-7-methyl- 4,5,6,7-tetrahydro-1H- pyrazolo[4,3-c]pyridine- 3-carboxamide | 605.5 |
| 686 | Ex. 567 | 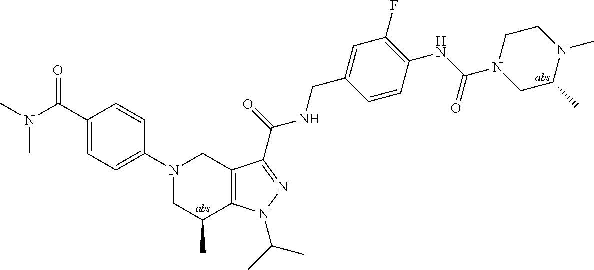 | (S)-5-(4- (dimethylcarbamoyl) phenyl)-N-(4-((R)-3,4- dimethylpiperazine-1- carboxamido)-3- fluorobenzyl)-1- isopropyl-7-methyl- 4,5,6,7-tetrahydro-1H- pyrazolo[4,3-c]pyridine- 3-carboxamide | 633.6 |
| 687 | Ex. 567 | 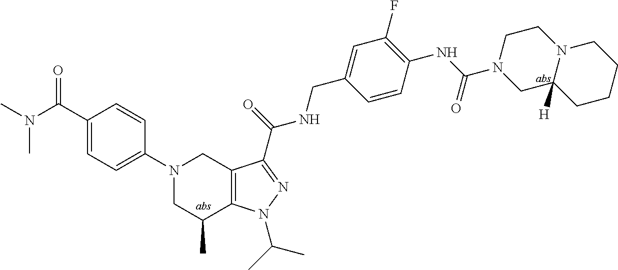 [SMILES: CC(C)n1nc(C(=O)NCc2ccc(NC(=O)N3CCN4CCCC[C@@H]4C3)c(F)c2)c2c1[C@@H](C)CN(c1ccc(C(=O)N(C)C)cc1)C2] | (R)-N-(4-(((S)-5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2- fluorophenyl)octahydro- 2H-pyrido[1,2- a]pyrazine-2- carboxamide | 659.6 |
| 688 | Ex. 485 | 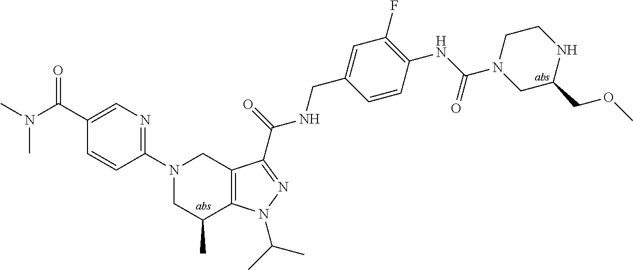 | (S)-5-(5- (dimethylcarbamoyl) pyridin-2-yl)-N-(3-fluoro-4- ((R)-3- (methoxymethyl) piperazine-1- carboxamido)benzyl)-1- isopropyl-7-methyl- 4,5,6,7-tetrahydro-1H- pyrazolo[4,3-c]pyridine- 3-carboxamide | 650.7 |
| 689 | Ex. 485 | 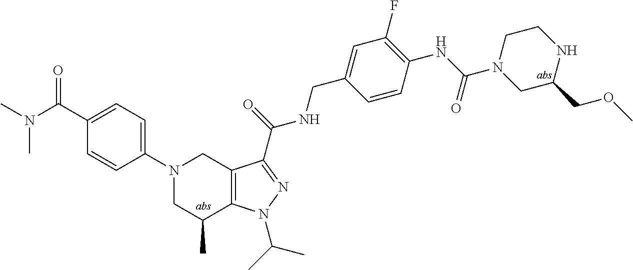 | (S)-5-(4- (dimethylcarbamoyl) phenyl)-N-(3-fluoro-4-((R)-3- (methoxymethyl) piperazine-1- carboxamido)benzyl)-1- isopropyl-7-methyl- 4,5,6,7-tetrahydro-1H- pyrazolo[4,3-c]pyridine- 3-carboxamide | 649.5 |
| 690 | Ex. 567 | 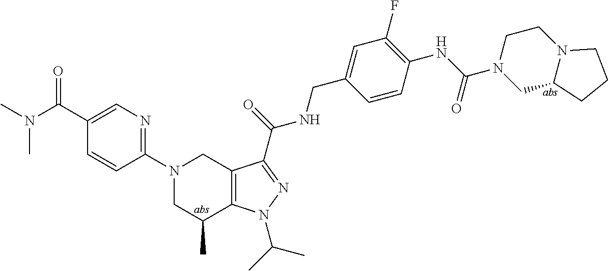 [SMILES: CC(C)n1nc(C(=O)NCc2ccc(NC(=O)N3CCN4CCC[C@@H]4C3)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- ((R)- octahydropyrrolo[1,2- a]pyrazine-2- carboxamido)benzyl)-1- isopropyl-7-methyl- 4,5,6,7-tetrahydro-1H- pyrazolo[4,3-c]pyridine- 3-carboxamide | 646.6 |
| 691 | Ex. 567 | 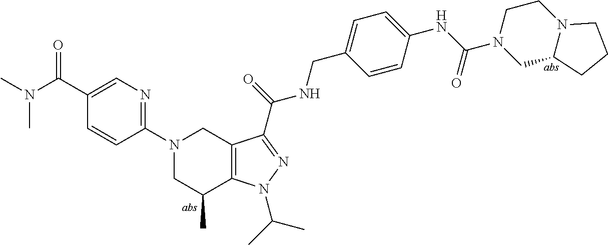 [SMILES: CC(C)n1nc(C(=O)NCc2ccc(NC(=O)N3CCN4CCC[C@@H]4C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-((R)- octahydropyrrolo[1,2- a]pyrazine-2- carboxamido)benzyl)- 4,5,6,7-tetrahydro-1H- pyrazolo[4,3-c]pyridine- 3-carboxamide | 628.5 |
| 692^{∧} | Ex. 546^{a} | 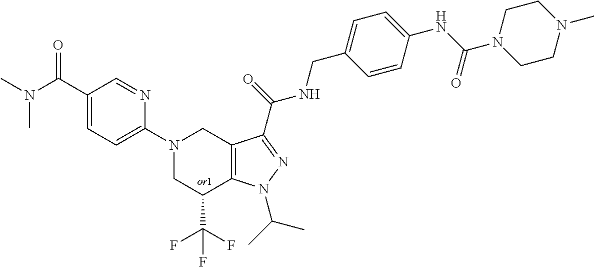 | rel-(R or S)-5-(5- (dimethylcarbamoyl) pyridin-2-yl)-1-isopropyl-N- (4-(4-methylpiperazine- 1-carboxamido)benzyl)- 7-(trifluoromethyl)- 4,5,6,7-tetrahydro-1H- pyrazolo[4,3-c]pyridine- 3-carboxamide | 656.4 |
| 693^{∧} | Ex. 546^{a} | 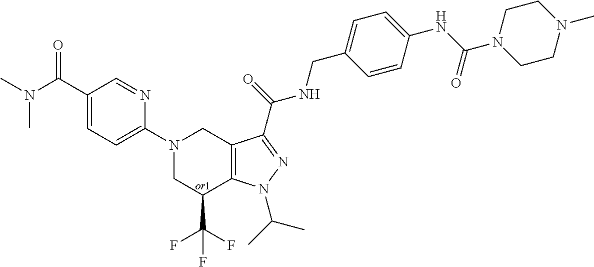 | rel-(R or S)-5-(5- (dimethylcarbamoyl) pyridin-2-yl)-1-isopropyl-N- (4-(4-methylpiperazine- 1-carboxamido)benzyl)- 7-(trifluoromethyl)- 4,5,6,7-tetrahydro-1H- pyrazolo[4,3-c]pyridine- 3-carboxamide | 656.4 |
| 694^{∧} | Ex. 4ⁱ | 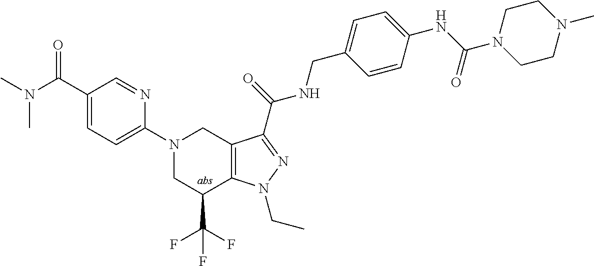 | rel-(R or S)-5-(5- (dimethylcarbamoyl) pyridin-2-yl)-1-ethyl-N-(4-(4- methylpiperazine-1- carboxamido)benzyl)-7- (trifluoromethyl)-4,5,6,7- tetrahydro-1H- pyrazolo[4,3-c]pyridine- 3-carboxamide | 642.3 |
| 695^{∧} | Ex. 4ⁱ | 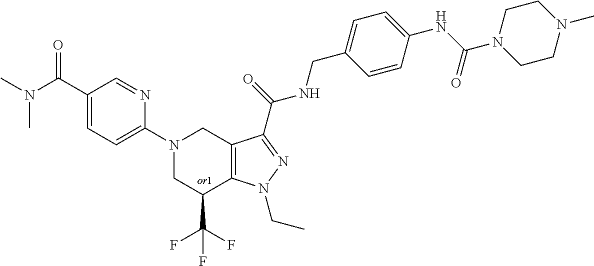 | rel-(R or S)-5-(5- (dimethylcarbamoyl) pyridin-2-yl)-1-ethyl-N-(4-(4- methylpiperazine-1- carboxamido)benzyl)-7- (trifluoromethyl)-4,5,6,7- tetrahydro-1H- pyrazolo[4,3-c]pyridine- 3-carboxamide | 642.3 |
| 696 | Ex. 4ⁱ | 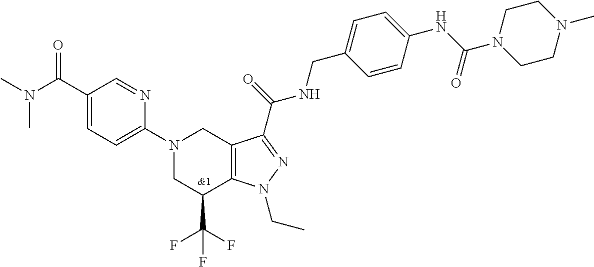 | rac-(R)-5-(5- (dimethylcarbamoyl) pyridin-2-yl)-1-ethyl-N-(4-(4- methylpiperazine-1- carboxamido)benzyl)-7- (trifluoromethyl)-4,5,6,7- tetrahydro-1H- pyrazolo[4,3-c]pyridine- 3-carboxamide | 642.4 |
| 697 | Ex. 567 | 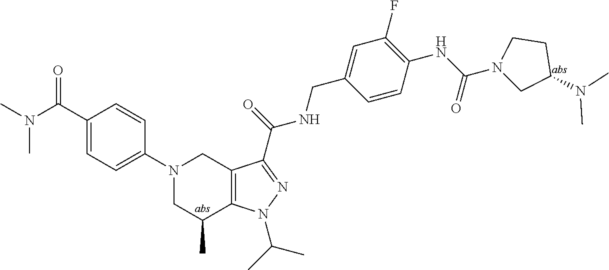 | (S)-N-(4-((S)-3- (dimethylamino)pyrrolidine- 1-carboxamido)-3- fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 633.5 |
| 698 | Ex. 128 | 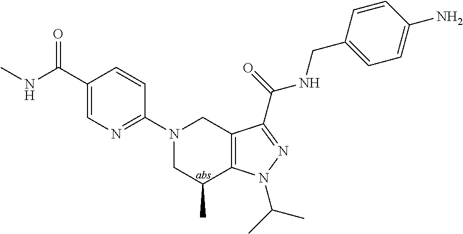 [SMILES: CNC(=O)c1ccc(N2Cc3c(C(=O)NCc4ccc(N)cc4)nn(C(C)C)c3[C@@H](C)C2)nc1] | (S)-N-(4-aminobenzyl)- 1-isopropyl-7-methyl-5- (5- (methylcarbamoyl)pyridin- 2-yl)-4,5,6,7- tetrahydro-1H- pyrazolo[4,3-c]pyridine- 3-carboxamide | 462.4 |
| 699 | Ex. 485 | 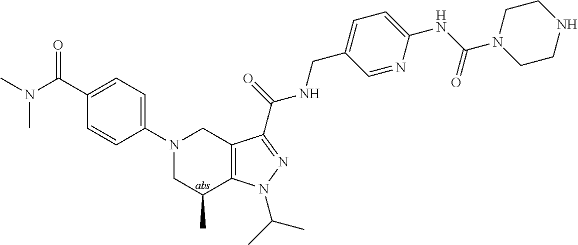 [SMILES: CC(C)n1nc(C(=O)NCc2ccc(NC(=O)N3CCNCC3)nc2)c2c1[C@@H](C)CN(c1ccc(C(=O)N(C)C)cc1)C2] | (S)-5-(4- (dimethylcarbamoyl) phenyl)-1-isopropyl-7- methyl-N-((6- (piperazine-1- carboxamido)pyridin-3- yl)methyl)-4,5,6,7- tetrahydro-1H- pyrazolo[4,3-c]pyridine- 3-carboxamide | 588.4 |
| 700 | Ex. 485 | 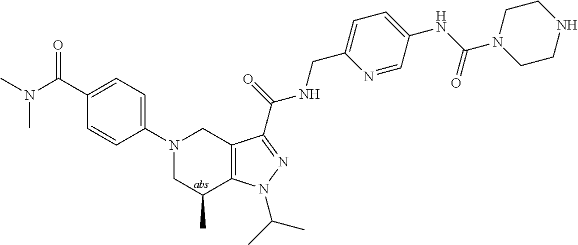 [SMILES: CC(C)n1nc(C(=O)NCc2ccc(NC(=O)N3CCNCC3)cn2)c2c1[C@@H](C)CN(c1ccc(C(=O)N(C)C)cc1)C2] | (S)-5-(4- (dimethylcarbamoyl) phenyl)-1-isopropyl-7- methyl-N-((5- (piperazine-1- carboxamido)pyridin-2- yl)methyl)-4,5,6,7- tetrahydro-1H- pyrazolo[4,3-c]pyridine- 3-carboxamide | 588.4 |
| 701 | Ex. 546ⁱ | 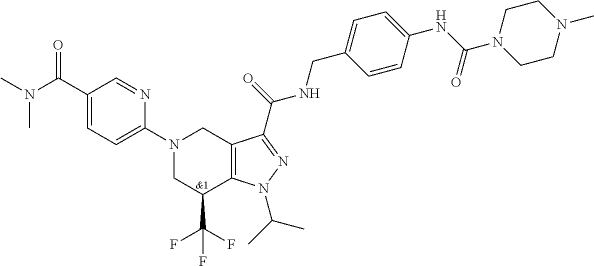 | rac-(R)-5-(5- (dimethylcarbamoyl) pyridin-2-yl)-1-isopropyl-N- (4-(4-methylpiperazine- 1-carboxamido)benzyl)- 7-(trifluoromethyl)- 4,5,6,7-tetrahydro-1H- pyrazolo[4,3-c]pyridine- 3-carboxamide | 656.4 |
| 702^{∧} | Ex. 4ⁱ | 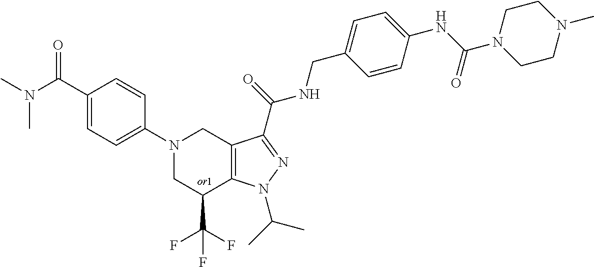 | rel-(R or S)-5-(4- (dimethylcarbamoyl) phenyl)-1-isopropyl-N-(4-(4- methylpiperazine-1- carboxamido)benzyl)-7- (trifluoromethyl)-4,5,6,7- tetrahydro-1H- pyrazolo[4,3-c]pyridine- 3-carboxamide | 655.3 |
| 703 | Ex. 4ⁱ | 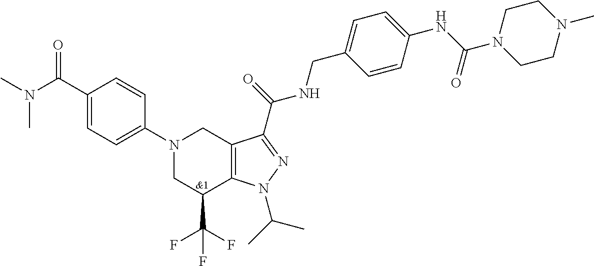 | rac-(R)-5-(4- (dimethylcarbamoyl) phenyl)-1-isopropyl-N-(4-(4- methylpiperazine-1- carboxamido)benzyl)-7- (trifluoromethyl)-4,5,6,7- tetrahydro-1H- pyrazolo[4,3-c]pyridine- 3-carboxamide | 655.3 |
| 704 | Ex. 485 | 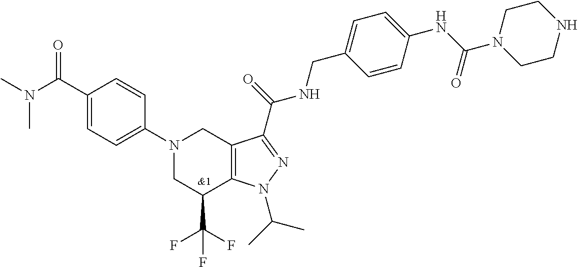 [SMILES: CC(C)n1nc(C(=O)NCc2ccc(NC(=O)N3CCNCC3)cc2)c2c1[C@@H](C(F)(F)F)CN(c1ccc(C(=O)N(C)C)cc1)C2] | rac-(R)-5-(4- (dimethylcarbamoyl) phenyl)-1-isopropyl-N-(4- (piperazine-1- carboxamido)benzyl)-7- (trifluoromethyl)-4,5,6,7- tetrahydro-1H- pyrazolo[4,3-c]pyridine- 3-carboxamide | 641.4 |
| 705^{∧} | Ex. 4ⁱ | 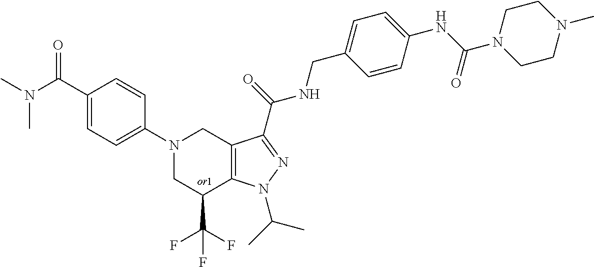 | rel-(R or S)-5-(4- (dimethylcarbamoyl) phenyl)-1-isopropyl-N-(4-(4- methylpiperazine-1- carboxamido)benzyl)-7- (trifluoromethyl)-4,5,6,7- tetrahydro-1H- pyrazolo[4,3-c]pyridine- 3-carboxamide | 655.3 |
| 706 | Ex 395^{fo} | 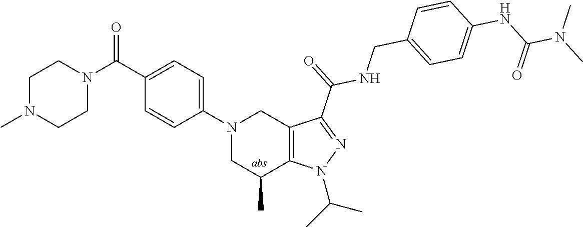 | (S)-N-(4-(3,3- dimethylureido)benzyl)- 1-isopropyl-7-methyl-5- (4-(4-methylpiperazine- 1-carbonyl)phenyl)- 4,5,6,7-tetrahydro-1H- pyrazolo[4,3-c]pyridine- 3-carboxamide | 601.5 |
| 707 | Ex. 128 | 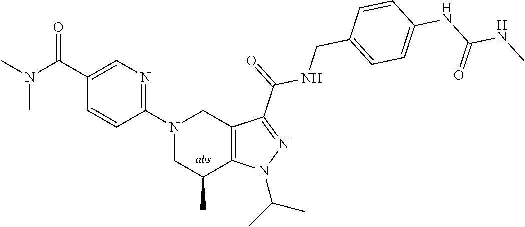 | (S)-5-(5- (dimethylcarbamoyl) pyridin-2-yl)-1-isopropyl-7- methyl-N-(4-(3- methylureido)benzyl)- 4,5,6,7-tetrahydro-1H- pyrazolo[4,3-c]pyridine- 3-carboxamide | 533.4 |
| 708 | Ex. 567 | 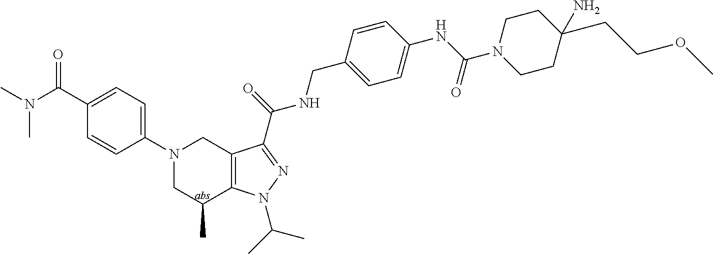 | (S)-N-(4-(4-amino-4-(2- methoxyethyl)piperidine- 1-carboxamido)benzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 659.5 |
| 709 | Ex. 567 | 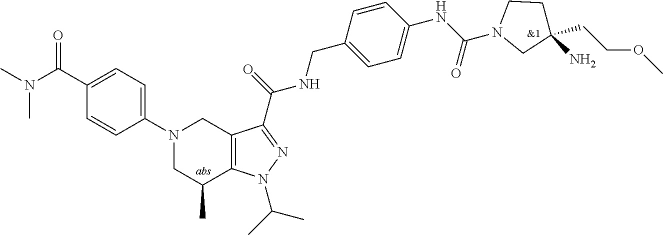 | (S)-N-(4-((RS)-3-amino- 3-(2- methoxyethyl)pyrrolidine- 1-carboxamido)benzyl)- 5-(4- (dimethylcarbamoyl) phenyl)-1-isopropyl-7- methyl-4,5,6,7- tetrahydro-1H- pyrazolo[4,3-c]pyridine- 3-carboxamide | 645.5 |
| 710 | Ex. 567 | 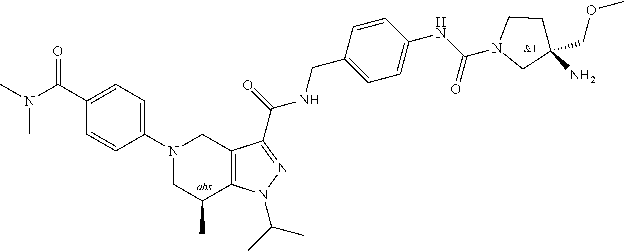 | (S)-N-(4-((RS)-3-amino- 3- (methoxymethyl)pyrrolidine- 1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 631.5 |
| 711 | Ex. 125^{q} | 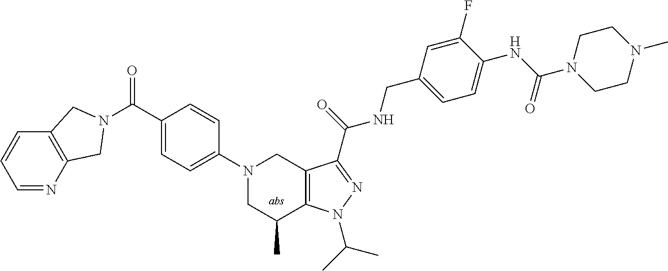 [SMILES: CC(C)n1nc(C(=O)NCc2ccc(NC(=O)N3CCN(C)CC3)c(F)c2)c2c1[C@@H](C)CN(c1ccc(C(=O)N3Cc4cccnc4C3)cc1)C2] | (S)-5-(4-(6,7-dihydro- 5H-pyrrolo[3,4- b]pyridine-6- carbonyl)phenyl)-N-(3- fluoro-4-(4- methylpiperazine-1- carboxamido)benzyl)-1- isopropyl-7-methyl- 4,5,6,7-tetrahydro-1H- pyrazolo[4,3-c]pyridine- 3-carboxamide | 694.5 |
| 712 | Ex. 567 | 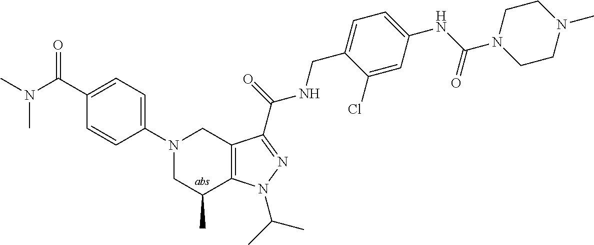 | (S)-N-(2-chloro-4-(4- methylpiperazine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 635.4 |
| 713^{∧} | Ex. 485 | 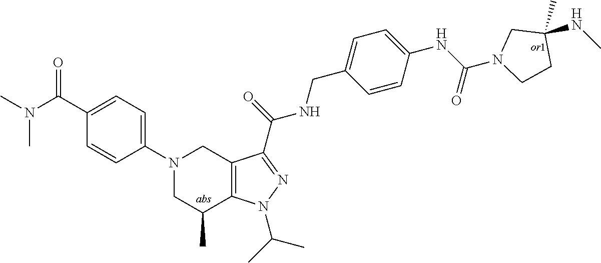 | (S)-5-(4- (dimethylcarbamoyl) phenyl)-1-isopropyl-7- methyl-N-(4-((R*)-3- methyl-3- (methylamino)pyrrolidine- 1-carboxamido)benzyl)- 4,5,6,7-tetrahydro-1H- pyrazolo[4,3-c]pyridine- 3-carboxamide | 615.4 |
| 714^{∧} | Ex. 485 | 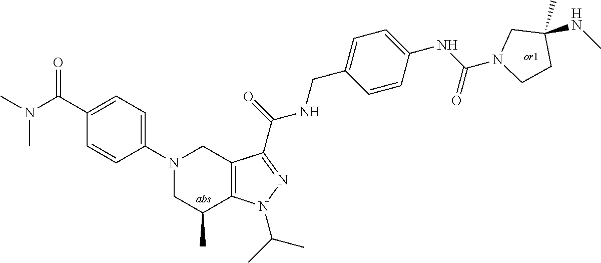 | (S)-5-(4- (dimethylcarbamoyl) phenyl)-1-isopropyl-7- methyl-N-(4-((R*)-3- methyl-3- (methylamino)pyrrolidine- 1-carboxamido)benzyl)- 4,5,6,7-tetrahydro-1H- pyrazolo[4,3-c]pyridine- 3-carboxamide | 616.3 |
| 715 | Ex. 128 | 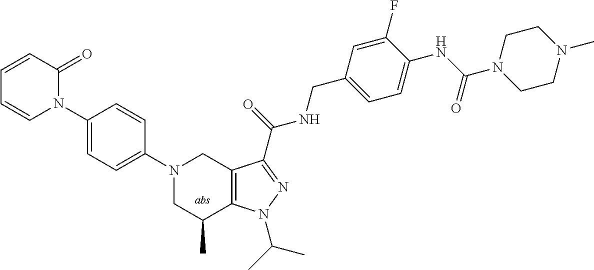 [SMILES: CC(C)n1nc(C(=O)NCc2ccc(NC(=O)N3CCN(C)CC3)c(F)c2)c2c1[C@@H](C)CN(c1ccc(-n3ccccc3=O)cc1)C2] | (S)-N-(3-fluoro-4-(4- methylpiperazine-1- carboxamido)benzyl)-1- isopropyl-7-methyl-5-(4- (2-oxopyridin-1(2H)- yl)phenyl)-4,5,6,7- tetrahydro-1H- pyrazolo[4,3-c]pyridine- 3-carboxamide | 641.7 |
| 716 | Ex. 567 | 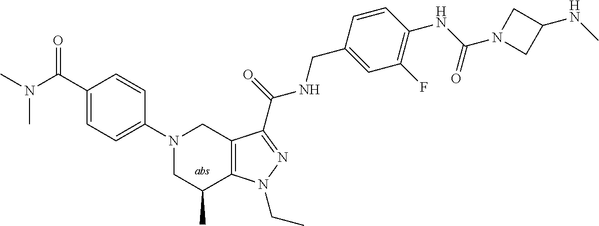 | (S)-5-(4- (dimethylcarbamoyl) phenyl)-1-ethyl-N-(3-fluoro- 4-(3- (methylamino)azetidine- 1-carboxamido)benzyl)- 7-methyl-4,5,6,7- tetrahydro-1H- pyrazolo[4,3-c]pyridine- 3-carboxamide | 591.5 |
| 717 | Ex. 567 | 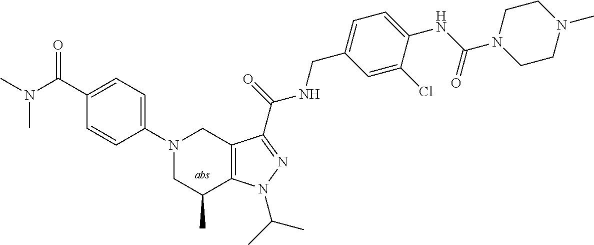 | (S)-N-(3-chloro-4-(4- methylpiperazine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 635.3 |
| 718 | Ex. 567 | 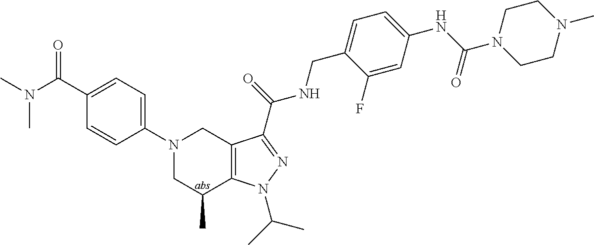 | (S)-5-(4- (dimethylcarbamoyl) phenyl)-N-(2-fluoro-4-(4- methylpiperazine-1- carboxamido)benzyl)-1- isopropyl-7-methyl- 4,5,6,7-tetrahydro-1H- pyrazolo[4,3-c]pyridine- 3-carboxamide | 619.5 |
| 719 | Ex. 127 | 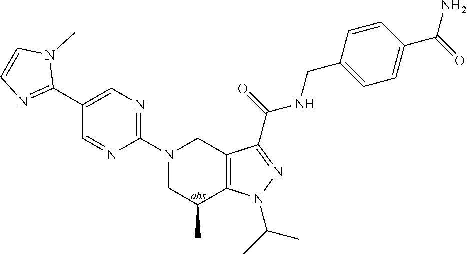 [SMILES: CC(C)n1nc(C(=O)NCc2ccc(C(N)=O)cc2)c2c1[C@@H](C)CN(c1ncc(-c3nccn3C)cn1)C2] | (S)-N-(4- carbamoylbenzyl)-1- isopropyl-7-methyl-5-(5- (1-methyl-1H-imidazol- 2-yl)pyrimidin-2-yl)- 4,5,6,7-tetrahydro-1H- pyrazolo[4,3-c]pyridine- 3-carboxamide | 514.3 |
| 720 | Ex. 485 | 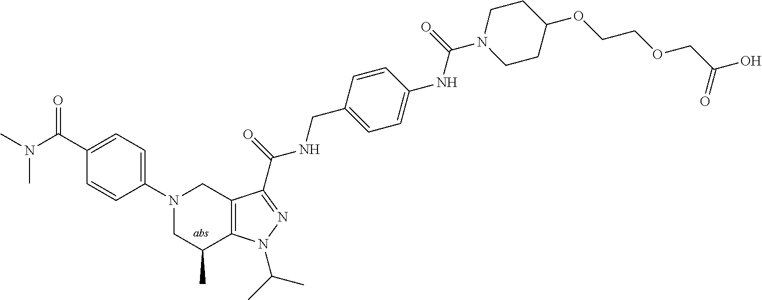 [SMILES: CC(C)n1nc(C(=O)NCc2ccc(NC(=O)N3CCC(OCCOCC(=O)O)CC3)cc2)c2c1[C@@H](C)CN(c1ccc(C(=O)N(C)C)cc1)C2] | (S)-2-(2-((1-((4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)carbamoyl)piperidin- 4-yl)oxy)ethoxy)acetic acid | 704.4 |
| 721 | | 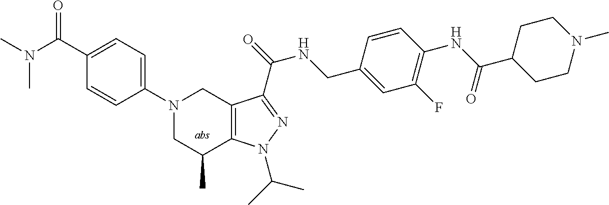 | (S)-5-(4- (dimethylcarbamoyl) phenyl)-N-(3-fluoro-4-(1- methylpiperidine-4- carboxamido)benzyl)-1- isopropyl-7-methyl- 4,5,6,7-tetrahydro-1H- pyrazolo[4,3-c]pyridine- 3-carboxamide | 618.5 |
| 722 | Ex. 485 | 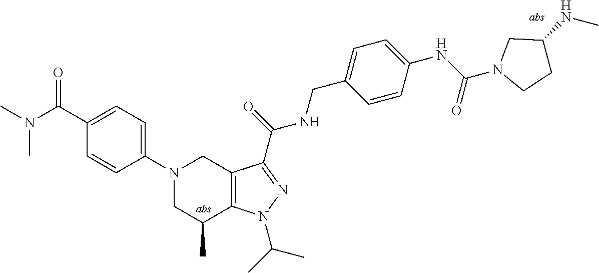 | (S)-5-(4- (dimethylcarbamoyl) phenyl)-1-isopropyl-7- methyl-N-(4-((R)-3- (methylamino)pyrrolidine- 1-carboxamido)benzyl)- 4,5,6,7-tetrahydro-1H- pyrazolo[4,3-c]pyridine- 3-carboxamide | 601.4 |
| 723 | Ex. 132^{otu} | 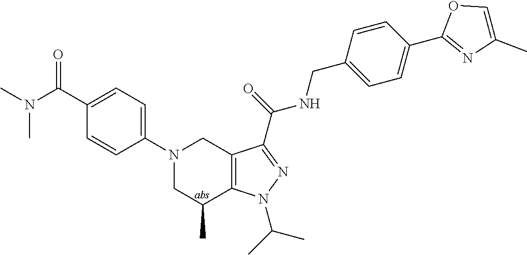 | (S)-5-(4- (dimethylcarbamoyl) phenyl)-1-isopropyl-7- methyl-N-(4-(4- methyloxazol-2- yl)benzyl)-4,5,6,7- tetrahydro-1H- pyrazolo[4,3-c]pyridine- 3-carboxamide | 541.3 |
| 724 | Ex. 132^{otu} | 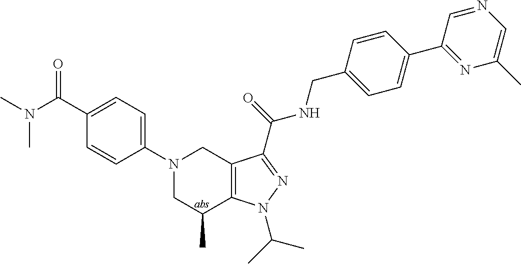 | (R)-5-(4- (dimethylcarbamoyl) phenyl)-1-isopropyl-7- methyl-N-(4-(6- methylpyrazin-2- yl)benzyl)-4,5,6,7- tetrahydro-1H- pyrazolo[4,3-c]pyridine- 3-carboxamide | 552.5 |
| 725 | Ex. 132^{otu} | 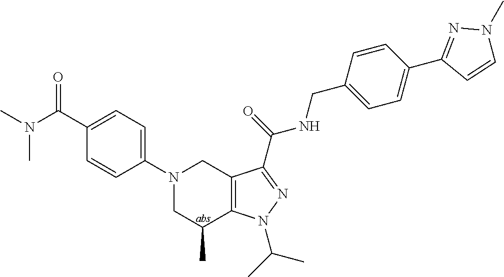 | (S)-5-(4- (dimethylcarbamoyl) phenyl)-1-isopropyl-7- methyl-N-(4-(1-methyl- 1H-pyrazol-3-yl)benzyl)- 4,5,6,7-tetrahydro-1H- pyrazolo[4,3-c]pyridine- 3-carboxamide | 540.4 |
| 726 | Ex 132^{otu} | 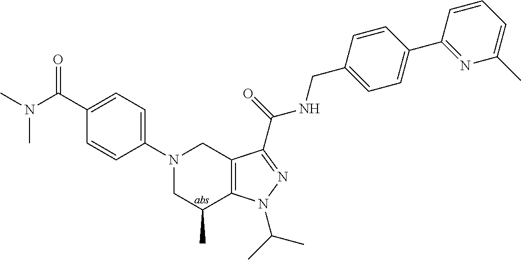 | (S)-5-(4- (dimethylcarbamoyl) phenyl)-1-isopropyl-7- methyl-N-(4-(6- methylpyridin-2- yl)benzyl)-4,5,6,7- tetrahydro-1H- pyrazolo[4,3-c]pyridine- 3-carboxamide | 551.3 |
| 727 | Ex. 132^{otu} | 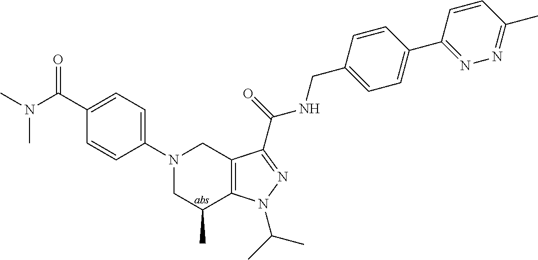 | (S)-5-(4- (dimethylcarbamoyl) phenyl)-1-isopropyl-7- methyl-N-(4-(6- methylpyridazin-3- yl)benzyl)-4,5,6,7- tetrahydro-1H- pyrazolo[4,3-c]pyridine- 3-carboxamide | 552.3 |
| 728 | Ex 132^{otu} | 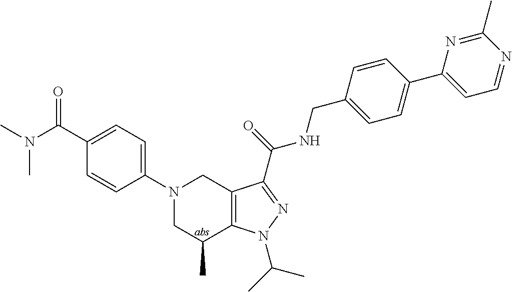 [SMILES: Cc1nccc(-c2ccc(CNC(=O)c3nn(C(C)C)c4c3CN(c3ccc(C(=O)N(C)C)cc3)C[C@@H]4C)cc2)n1] | (S)-5-(4- (dimethylcarbamoyl) phenyl)-1-isopropyl-7- methyl-N-(4-(2- methylpyrimidin-4- yl)benzyl)-4,5,6,7- tetrahydro-1H- pyrazolo[4,3-c]pyridine- 3-carboxamide | 552.5 |
| 729 | Ex. 132^{otu} | 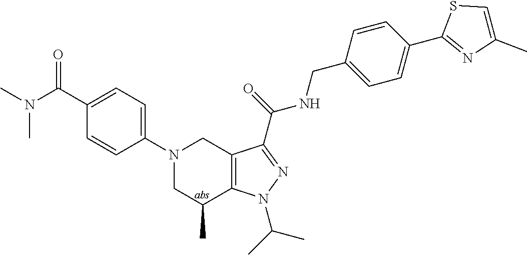 | (S)-5-(4- (dimethylcarbamoyl) phenyl)-1-isopropyl-7- methyl-N-(4-(4- methylthiazol-2- yl)benzyl)-4,5,6,7- tetrahydro-1H- pyrazolo[4,3-c]pyridine- 3-carboxamide | 557.4 |
| 730 | Ex. 132^{otu} | 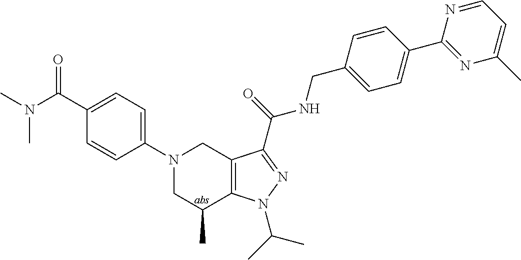 | (S)-5-(4- (dimethylcarbamoyl) phenyl)-1-isopropyl-7- methyl-N-(4-(4- methylpyrimidin-2- yl)benzyl)-4,5,6,7- tetrahydro-1H- pyrazolo[4,3-c]pyridine- 3-carboxamide | 552.4 |
| 731 | Ex 132^{otu} | 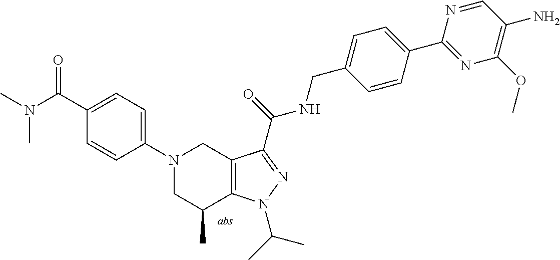 [SMILES: COc1nc(-c2ccc(CNC(=O)c3nn(C(C)C)c4c3CN(c3ccc(C(=O)N(C)C)cc3)C[C@@H]4C)cc2)ncc1N] | (S)-N-(4-(5-amino-4- methoxypyrimidin-2- yl)benzyl)-5-(4- (dimethylcarbamoyl) phenyl)-1-isopropyl-7- methyl-4,5,6,7- tetrahydro-1H- pyrazolo[4,3-c]pyridine- 3-carboxamide | 583.5 |
| 732 | Ex. 132^{otu} | 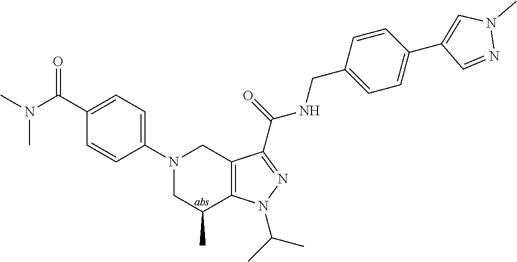 | (S)-5-(4- (dimethylcarbamoyl) phenyl)-1-isopropyl-7- methyl-N-(4-(1-methyl- 1H-pyrazol-4-yl)benzyl)- 4,5,6,7-tetrahydro-1H- pyrazolo[4,3-c]pyridine- 3-carboxamide | 540.5 |
| 733 | Ex. 567 | 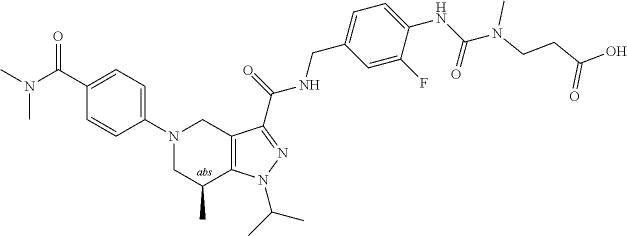 [SMILES: CC(C)n1nc(C(=O)NCc2ccc(NC(=O)N(C)CCC(=O)O)c(F)c2)c2c1[C@@H](C)CN(c1ccc(C(=O)N(C)C)cc1)C2] | (S)-3-(3-(4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2-fluorophenyl)-1- methylureido)propanoic acid | 622.5 |
| 734 | Ex. 567 | 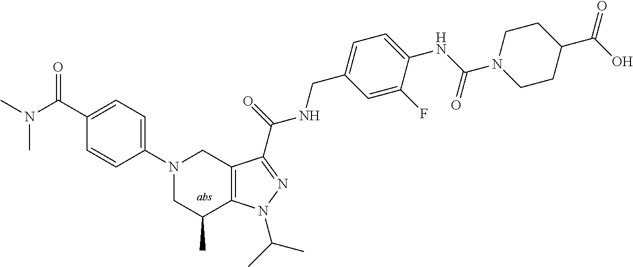 [SMILES: CC(C)n1nc(C(=O)NCc2ccc(NC(=O)N3CCC(C(=O)O)CC3)c(F)c2)c2c1[C@@H](C)CN(c1ccc(C(=O)N(C)C)cc1)C2] | (S)-1-((4-((5-(4- (dimethylcarbamoyl) phenyl)-1-isopropyl-7- methyl-4,5,6,7- tetrahydro-1H- pyrazolo[4,3-c]pyridine- 3-carboxamido)methyl)- 2- fluorophenyl)carbamoyl) piperidine-4-carboxylic acid | 648.5 |
| 735 | Ex. 567 | 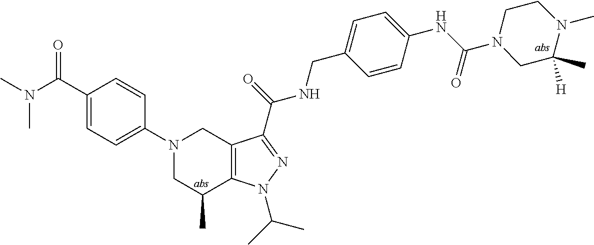 | (S)-5-(4- (dimethylcarbamoyl) phenyl)-N-(4-((S)-3,4- dimethylpiperazine-1- carboxamido)benzyl)-1- isopropyl-7-methyl- 4,5,6,7-tetrahydro-1H- pyrazolo[4,3-c]pyridine- 3-carboxamide | 615.5 |
| 736 | Ex. 567 | 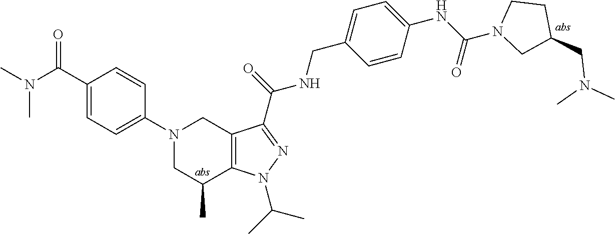 | (S)-N-(4-((S)-3- ((dimethylamino)methyl) pyrrolidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 629.5 |
| 737 | Ex. 567 | 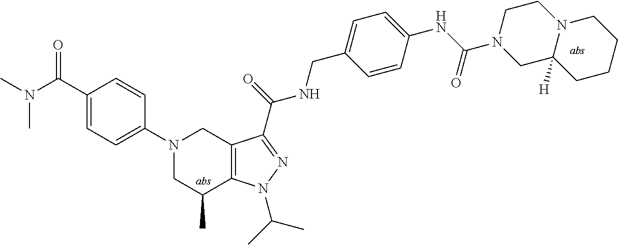 [SMILES: CC(C)n1nc(C(=O)NCc2ccc(NC(=O)N3CCN4CCCC[C@H]4C3)cc2)c2c1[C@@H](C)CN(c1ccc(C(=O)N(C)C)cc1)C2] | (S)-N-(4-(((S)-5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)octahydro-2H- pyrido[1,2-a]pyrazine-2- carboxamide | 641.5 |
| 738 | Ex 567 | 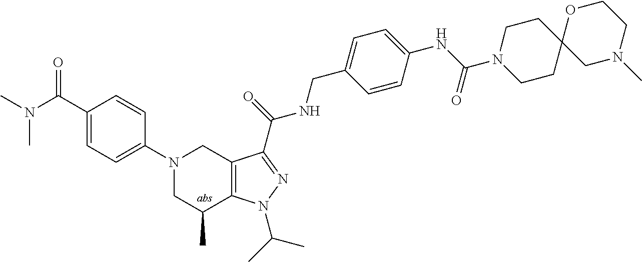 | (S)-N-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)-4-methyl-1-oxa- 4,9- diazaspiro[5.5]undecane- 9-carboxamide | 671.5 |
| 739 | Ex 567 | 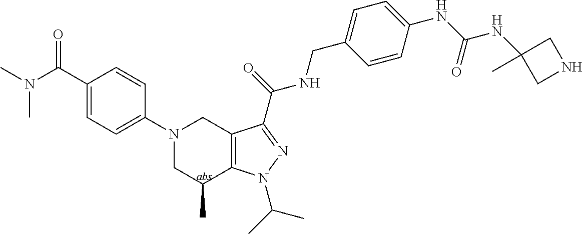 [SMILES: CC(C)n1nc(C(=O)NCc2ccc(NC(=O)NC3(C)CNC3)cc2)c2c1[C@@H](C)CN(c1ccc(C(=O)N(C)C)cc1)C2] | (S)-5-(4- (dimethylcarbamoyl) phenyl)-1-isopropyl-7- methyl-N-(4-(3-(3- methylazetidin-3- yl)ureido)benzyl)- 4,5,6,7-tetrahydro-1H- pyrazolo[4,3-c]pyridine- 3-carboxamide | 587.4 |
| 740 | Ex 567 | 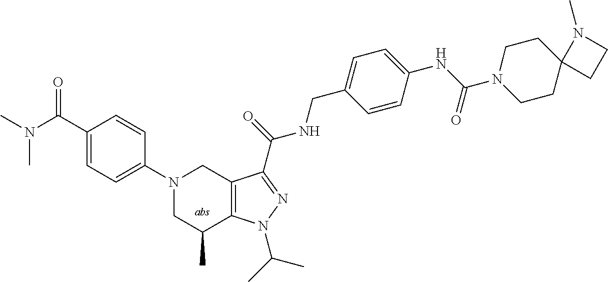 [SMILES: CC(C)n1nc(C(=O)NCc2ccc(NC(=O)N3CCC4(CC3)CCN4C)cc2)c2c1[C@@H](C)CN(c1ccc(C(=O)N(C)C)cc1)C2] | (S)-5-(4- (dimethylcarbamoyl) phenyl)-1-isopropyl-7- methyl-N-(4-(1-methyl- 1,7- diazaspiro[3.5]nonane- 7-carboxamido)benzyl)- 4,5,6,7-tetrahydro-1H- pyrazolo[4,3-c]pyridine- 3-carboxamide | 641.5 |
| 741 | Ex. 567 | 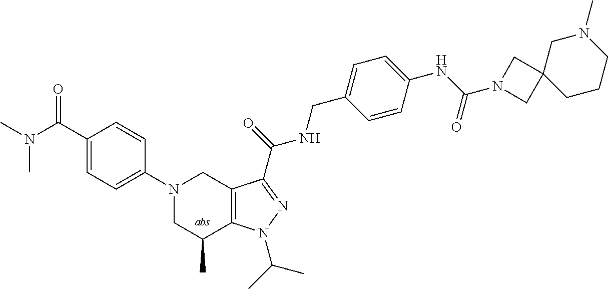 [SMILES: CC(C)n1nc(C(=O)NCc2ccc(NC(=O)N3CC4(CCCN(C)C4)C3)cc2)c2c1[C@@H](C)CN(c1ccc(C(=O)N(C)C)cc1)C2] | (S)-5-(4- (dimethylcarbamoyl) phenyl)-1-isopropyl-7- methyl-N-(4-(6-methyl- 2,6- diazaspiro[3.5]nonane- 2-carboxamido)benzyl)- 4,5,6,7-tetrahydro-1H- pyrazolo[4,3-c]pyridine- 3-carboxamide | 641.5 |
| 742 | Ex. 567 | 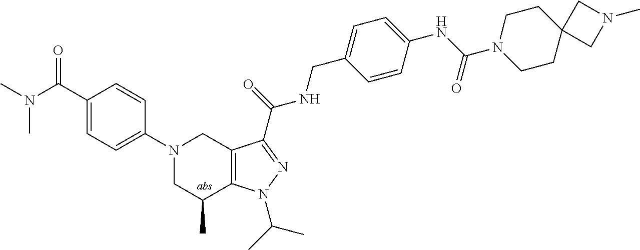 | (S)-5-(4- (dimethylcarbamoyl) phenyl)-1-isopropyl-7- methyl-N-(4-(2-methyl- 2,7- diazaspiro[3.5]nonane- 7-carboxamido)benzyl)- 4,5,6,7-tetrahydro-1H- pyrazolo[4,3-c]pyridine- 3-carboxamide | 641.5 |
| 743 | Ex. 567 | 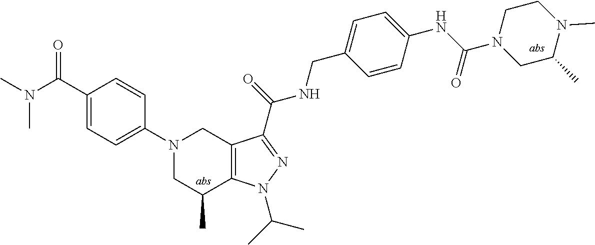 | (S)-5-(4- (dimethylcarbamoyl) phenyl)-N-(4-((R)-3,4- dimethylpiperazine-1- carboxamido)benzyl)-1- isopropyl-7-methyl- 4,5,6,7-tetrahydro-1H- pyrazolo[4,3-c]pyridine- 3-carboxamide | 615.5 |
| 744 | Ex. 567 | 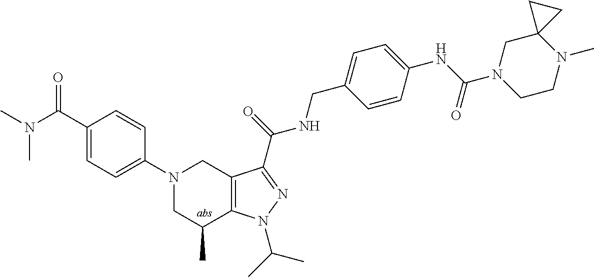 | (S)-5-(4- (dimethylcarbamoyl) phenyl)-1-isopropyl-7- methyl-N-(4-(4-methyl- 4,7- diazaspiro[2.5]octane-7- carboxamido)benzyl)- 4,5,6,7-tetrahydro-1H- pyrazolo[4,3-c]pyridine- 3-carboxamide | 627.5 |
| 745 | Ex. 567 | 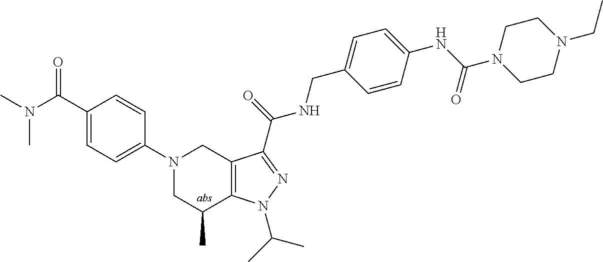 [SMILES: CCN1CCN(C(=O)Nc2ccc(CNC(=O)c3nn(C(C)C)c4c3CN(c3ccc(C(=O)N(C)C)cc3)C[C@@H]4C)cc2)CC1] | (S)-5-(4- (dimethylcarbamoyl) phenyl)-N-(4-(4- ethylpiperazine-1- carboxamido)benzyl)-1- isopropyl-7-methyl- 4,5,6,7-tetrahydro-1H- pyrazolo[4,3-c]pyridine- 3-carboxamide | 615.5 |
| 746 | Ex. 567 | 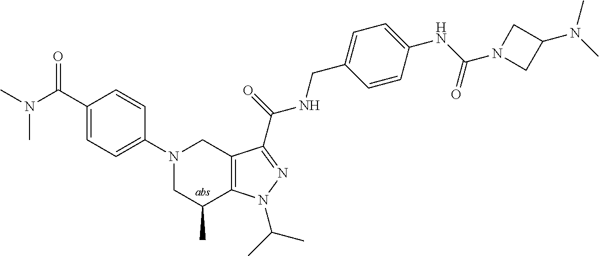 | (S)-N-(4-(3- (dimethylamino) azetidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 601.5 |
| 747 | Ex. 567 | 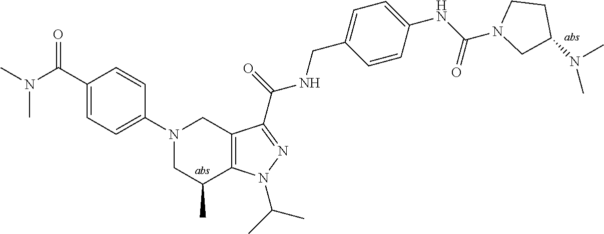 | (S)-N-(4-((S)-3- (dimethylamino) pyrrolidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 615.5 |
| 748 | Ex. 567 | 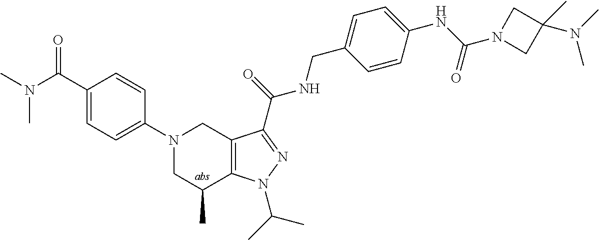 | (S)-N-(4-(3- (dimethylamino)-3- methylazetidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 615.5 |
| 749 | Ex. 567 | 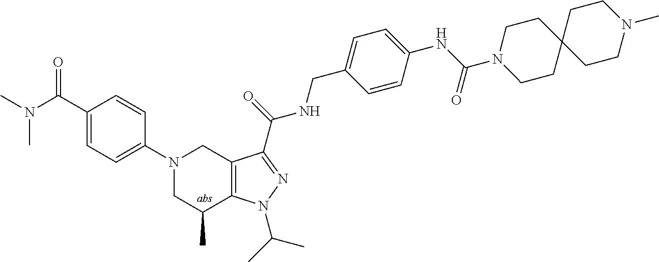 | (S)-N-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)-9-methyl-3,9- diazaspiro[5.5]undecane- 3-carboxamide | 669.6 |
| 750 | Ex. 567 | 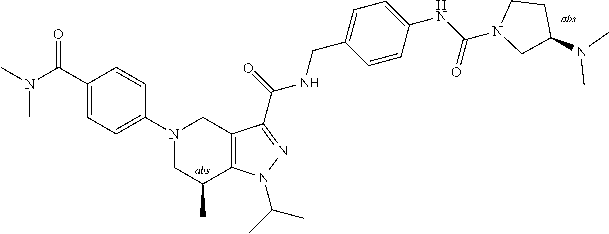 | (S)-N-(4-((R)-3- (dimethylamino)pyrrolidine- 1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 615.6 |
| 751 | Ex. 567 | 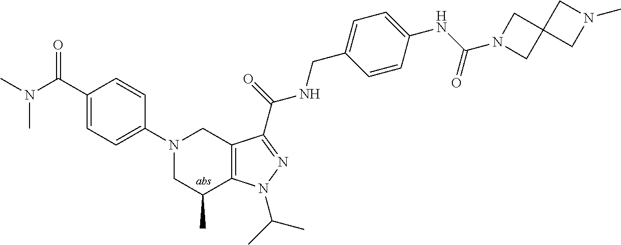 | (S)-5-(4- (dimethylcarbamoyl) phenyl)-1-isopropyl-7- methyl-N-(4-(6-methyl- 2,6- diazaspiro[3.3]heptane- 2-carboxamido)benzyl)- 4,5,6,7-tetrahydro-1H- pyrazolo[4,3-c]pyridine- 3-carboxamide | 613.5 |
| 752 | Ex. 567 | 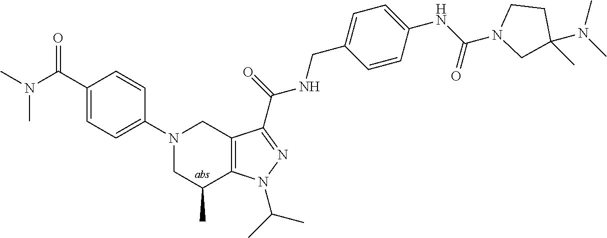 | (7S)-N-(4-(3- (dimethylamino)-3- methylpyrrolidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 629.5 |
| 753 | Ex. 567 | 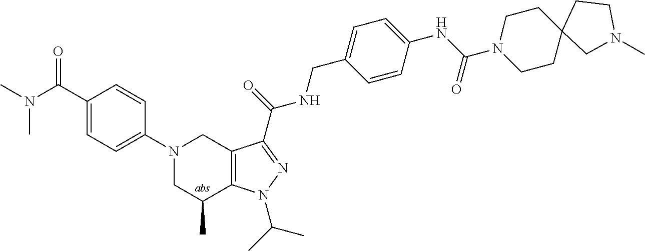 | (S)-N-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)-2-methyl-2,8- diazaspiro[4.5]decane- 8-carboxamide | 655.5 |
| 754 | Ex. 567 | 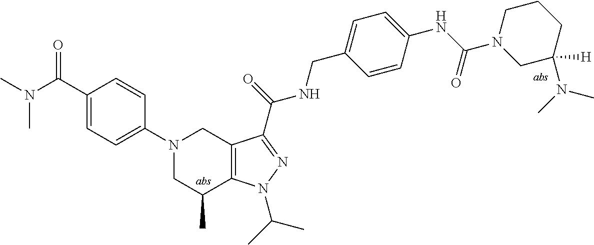 | (S)-N-(4-((R)-3- (dimethylamino) piperidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 629.5 |
| 755 | Ex. 567 | 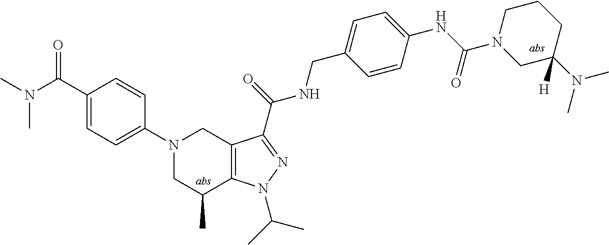 | (S)-N-(4-((S)-3- (dimethylamino) piperidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 629.5 |
| 756 | Ex. 567 | 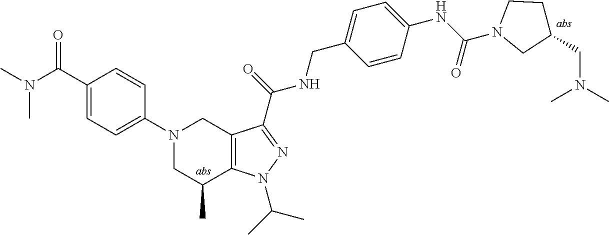 | (S)-N-(4-((R)-3- ((dimethylamino)methyl) pyrrolidine-1- carboxamido)benzyl)-5- (4- (dimethylcarbamoyl) phenyl)-1-isopropyl-7- methyl-4,5,6,7- tetrahydro-1H- pyrazolo[4,3-c]pyridine- 3-carboxamide | 629.5 |
| 757 | Ex. 567 | 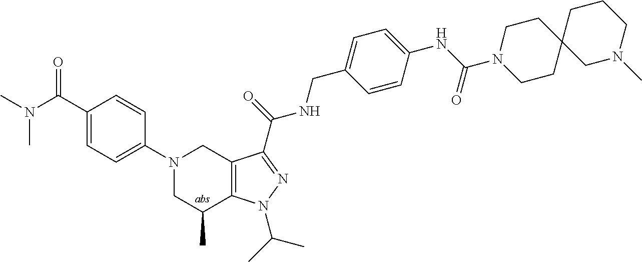 | (S)-N-(4-((5-(4- (dimethylcarbamoyl) phenyl)-1-isopropyl-7- methyl-4,5,6,7- tetrahydro-1H- pyrazolo[4,3-c]pyridine- 3- carboxamido)methyl) phenyl)-2-methyl-2,9- diazaspiro[5.5]undecane- 9-carboxamide | 669.5 |
| 758 | Ex. 567 | 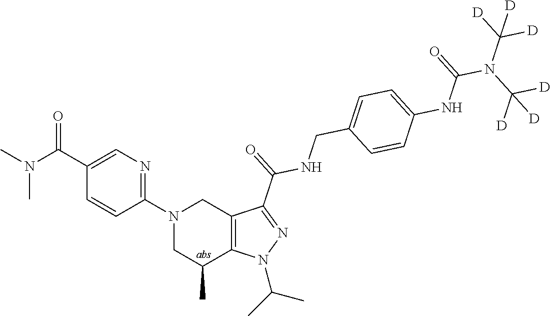 [SMILES: [2H]C([2H])([2H])N(C(=O)Nc1ccc(CNC(=O)c2nn(C(C)C)c3c2CN(c2ccc(C(=O)N(C)C)cn2)C[C@@H]3C)cc1)C([2H])([2H])[2H]] | (S)-N-(4-(3,3-bis(methyl- d₃)ureido)benzyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 553.6 |

¹H NMR* = analytical data was obtained as the hydrochloride, trifluoroacetate or formate salt form;
Example #^{∧} = chiral purification data listed below
^{a}: Amide coupling altered: At 0° C under nitrogen gas, to a stirred solution of amine (1.00 equivalent), acid (1.00 equivalent), HATU (1.05 equivalents) in DMF (0.2 M) was added DIEA (4.50 equivalents). The reaction mixture was stirred at room temperature for 2 h then purified by reverse phase HPLC and lyophilized to give the desired target.
^{b}: SNAr altered: A reaction mixture of amine (1.0 equivalent), fluoro-substituted heterocycle (1.3 equivalents), K₂CO₃ (3.0 equivalents) in DMF (0.15M) was stirred at 130° C for 21 h. The suspension was concentrated in vacuo then purified by column chromatography (silica gel, 0-40% EtOAc: PE) to give the desired target.
^{c}: Copper(I)-catalyzed cross coupling altered: To 6,7-membered ring lactams (2.0 equivalents) was added a solution of iodo-substituted heterocycle (1.0 equivalents) in dioxane (0.1M) followed by CuI (0.1 equivalent), DMEDA (0.2 equivalent), then Cs₂CO₃ (3.0 equivalents) under nitrogen gas. The reaction mixture was stirred at 120° C for 16 h then concentrated in vacuo. The residue was purified by preparative TLC to give the desired target.
^{d}: Amide Coupling altered: To a stirred solution of acid (1.0 equivalent), amine (1.2 equivalent), EDCl (1.4 equivalents), HOBt (1.2 equivalents) in ACN (0.13 M) was added DIEA (3.0 equivalents). The reaction mixture was stirred at room temperature for ~24 h then diluted with H₂O and NH₄Cl (50 mL). The suspension was extracted with DCM (2 x 50 mL). The combined organic layer was concentrated in vacuo to give the desired target.
^{e}: SNAr altered: A reaction mixture of amine (1.0 equivalent) and fluoro-substituted heterocycle (2.0 equivalents) in H₂O (0.56M) was added DIEA (0.47 mL). The reaction mixture was stirred at 100° C for 24 h then cooled to room temperature and stirred at room temperature for 72 h. The suspension was quenched with 1M citric acid to pH = 3 then extracted with DCM (3x). The organic layers were combined then dried with Na₂SO₄ and concentrated in vacuo to give the desired target.
^{f}: Palladium coupling altered: A reaction mixture of amine (1.00 equivalent), chloro-substituted heterocycle (3.45 equivalents) and K₃PO₄ (3.00 equivalents) in tert-amyl alcohol (0.1 M) was degassed with nitrogen gas for 5 min before RuPhos Pd G4 (0.100 equivalent) was added. The suspension was degassed with nitrogen gas again for 5 min then stirred at 100° C for 17 h. The reaction mixture was concentrated via nitrogen gas then dissolved in MeOH and filtered. The filtrate was diluted with EtOAc and filtered through Celite. The filter cake was rinsed with EtOAc. The filtrate was concentrated in vacuo. The residue was dissolved in EtOAc and heptane then concentrated in vacuo (2x). The residue was dissolved in minimal CHCl₃ and purified by column chromatography (silica gel, 0-100% EtOAc: heptane) to give the desired target.
^{g}: Palladium coupling altered: To a reaction mixture of amine (1.00 equivalent), chloro-substituted heterocycle (1.20 equivalents) and Cs₂CO₃ (3.00 equivalents) in toluene (0.06 M) was added BINAP (0.10 equivalent) and Pd₂(dba)₃ (0.05 equivalent) under nitrogen gas. The reaction mixture was stirred at 110° C for 16 h then filtered. The filter cake was washed with THF (10 mL) and DCM (10 mL). The filtrate was concentrated in vacuo then purified by column chromatography (silica gel, 0:0:100 to 10:90:0 of MeOH: EtOAc: PE) to give the desired target.
h: Palladium coupling altered: To a reaction mixture of chloro-substituted heterocycle (1.1 equivalents), amine (1.0 equivalent), K₃PO₄ (3.0 equivalents) and 4MetBuXPhos Pd G3 (0.10 equivalent) under nitrogen was added tert-amyl alcohol (0.3 M). The suspension was stirred at 90° C for 3 h then concentrated in vacuo. The residue was dissolved in DCM then filtered. The filtrate was purified by column chromatography (silica gel, 0-10% MeOH: DCM) to give the desired target.
ⁱ: Palladium Coupling altered: A reaction mixture of amine (1.00 equivalent), chloro-substituted heterocycle (1.25 equivalents) and K₃PO₄ (3.00 equivalents) in tert-amyl alcohol (0.1 M) was degassed with nitrogen gas for 5 min before RuPhos Pd G3 (0.102 equivalent) was added. The suspension was degassed with nitrogen gas again for 5 min then stirred at 100° C for 20 h and at room temperature for ~72 h. The reaction mixture was concentrated via nitrogen gas then dissolved in MeOH and filtered. The filtrate was poured into stirring H₂O and diluted with EtOAc. The aqueous layer was back-extracted with EtOAc (2 × 7 mL). The combined organic layer was washed with brine and dried with Na₂SO₄. The suspension was filtered, concentrated in vacuo and dissolved in DCM and minimal CHCl₃. The solution was purified by column chromatography (silica gel, 0-100% EtOAc: heptane) to give the desired target.
^{j}: SNAr altered: A reaction mixture of amine (1.0 equivalent), fluoro-substituted heterocycle (1.2 equivalents) and CsF (5.0 equivalents) in DMSO (0.1 M) was stirred at 130° C for 48 h then an additional portion of fluoro-substituted heterocycle (2.4 equivalents) was added. The reaction mixture was stirred at 130° C for 24 h then lyophilized to give the desired target.
^{k}: SNAr altered: To a solution of amine (1.0 equivalent) in n-butyl acetate (0.3 M) was added chloro-substituted heterocycle (1.2 equivalents) and K₃PO₄ (3.0 equivalents). The reaction mixture was stirred at 115° C for 2 h then diluted with ACN and filtered. The filtrate was concentrated in vacuo to give the desired target.
^{l}: Palladium Coupling altered: A reaction mixture of amine (1.00 equivalent), chloro-substituted heterocycle (1.40 equivalents), K₃PO₄ (3.00 equivalents) and RuPhos Pd G3 (0.08 equivalent) were degassed with nitrogen gas then tert-amyl alcohol (0.3 M) was added. The suspension was stirred at 90° C for 2 h then 4MetBuXPhos Pd G3 (0.10 equivalent). The reaction mixture was stirred at 90° C for 1 h then concentrated in vacuo. The residue was dissolved in DCM and filtered. The filtrate was purified by column chromatography (silica gel, 0-10% MeOH: DCM) to give the desired target.
^{m}: Addition of deprotection after the SNAr: At 0° C under nitrogen gas, to a solution of ether (1.0 equivalent) in DCM (0.2 M) was added 2M BBr₃ in DCM (2.0 equivalents) then stirred at room temperature for 16 h. At 0° C, an additional portion of 2M BBr₃ in DCM (1.4 equivalents) was added. The reaction mixture was stirred at room temperature for 16 h. The suspension was quenched with H₂O (4 mL) at 0° C then basified with 15% aqueous NaOH to pH~8. The basic mixture was extracted with EtOAc (4 × 20 mL). The combined organic layer was dried over Na₂SO₄, filtered and concentrated in vacuo to give the desired target.
ⁿ: Addition of methylation after the lactam cyclization: To a reaction mixture of lactam (1.0 equivalent) in ACN (0.1 M) and DMF (0.1 M) was added Cs₂CO₃ (3.0 equivalents). The suspension was stirred at 60º C for 1 h then MeI (13.0 equivalents) was added. The reaction mixture was stirred at 60° C for 16 h then concentrated in vacuo. The residue was purified by reverse phase HPLC (C₁₈ 150 mm × 30 mm × 5 μm column, Mobile Phase A: H₂O (0.05% formic acid)/Mobile Phase B: ACN, 21 to 61% of Mobile Phase B over 9 min then held at 100% of Mobile Phase B for 2 min; flow rate = 30 mL/min) to give the desired target.
^{o}: Amide Coupling altered: A reaction mixture of acid (1.0 equivalent), amine (1.3 equivalent), EDCl (1.5 equivalents), HOPO (1.2 equivalents) and TEA (4.0 equivalents) in ACN (0.08 M) were stirred at room temperature for ~24 h then diluted with H₂O (2 mL). The suspension was extracted with DCM (3 × 2 mL). The combined organic layer was dried with Na₂SO₄ and concentrated in vacuo. The residue was suspended in DMSO then filtered and purified by reverse phase HPLC (Sunfire C₁₈ 100 mm × 19 mm × 5 μm column, Mobile Phase A: H₂O/Mobile Phase B: ACN (0.05% TFA), 25 to 95% of Mobile Phase B over 10.0 min, 95% of Mobile Phase B hold 1.0 min, flow rate: 25 mL/min) to provide to give the desired target.
^{p}: Addition of deprotection in final step: A reaction solution of BOC-protected amine (1.0 equivalent) in DCM (0.07 M) was added TFA (0.3 M). The suspension was stirred at room temperature for 20 min then concentrated in vacuo. The residue was dissolved in DMSO then filtered and purified by reverse phase HPLC (Sunfire C₁₈ 100 mm × 19 mm × 5 μm column, Mobile Phase A: H₂O/Mobile Phase B: ACN (0.05% TFA), 60 to 80% of Mobile Phase B over 10.0 min, 95% of Mobile Phase B hold 1.0 min, flow rate: 25 mL/min) to provide to give the desired target.
^{q}: Palladium Coupling altered: Under nitrogen gas, a reaction mixture of acid (1.0 equivalent), bromo-substituted heterocycle (1.1 equivalents), KOTMS (3.2 equivalents) and P(t-Bu)₃ Pd G2 (0.1 equivalent) in tert-amyl alcohol (0.05 M). The suspension was degassed for 5 min then stirred at 60° C for 90 min then phosphoric acid (4.0 equivalents) was added. The reaction mixture was diluted with H₂O (3 mL) to pH = 3 then extracted with EtOAc (2 × 3 mL). The combined organic layers were dried with Na₂SO₄ and concentrated in vacuo. The residue was suspended in heptane, sonicated then filtered. The filter cake was washed with heptane then dried further under high vacuo. The solid was collected to provide to give the desired target.
^{r}: Addition of hydrolysis in last step: To an ester (1.0 equivalent) in ACN (0.03 M) was added NaOH (5.0 equivalents) and H₂O (0.11 M). The suspension was stirred at 40° C for 25 min then concentrated in vacuo. The residue was dissolved in DMSO then filtered and purified by reverse phase HPLC (Sunfire C₁₈ 100 mm × 19 mm × 5 μm column, Mobile Phase A: H₂O/Mobile Phase B: ACN (0.05% TFA), 55 to 75% of Mobile Phase B over 10.0 min, 95% of Mobile Phase B hold 1.0 min, flow rate: 25 mL/min) to provide to give the desired target.
^{s}: Additional Amide Coupling in last step: A reaction mixture of amine (1.0 equivalent), acid (1.1 equivalent), chloro-N,N,N′,N′-tetramethylformamidinium hexafluorophosphate (1.5 equivalents) and 1-methylimidazole (3.5 equivalents) in ACN (0.08 M) were stirred at room temperature for ~24 h then diluted with H₂O (1 mL). The suspension was extracted with DCM (3 × 2 mL). The combined organic layer was dried with Na₂SO₄ and concentrated in vacuo. The residue was suspended in DMSO then filtered and purified by reverse phase HPLC (Sunfire C₁₈ 100 mm × 19 mm × 5 μm column, Mobile Phase A: H₂O/Mobile Phase B: ACN (0.05% TFA), 60 to 80% of Mobile Phase B over 10.0 min, 95% of Mobile Phase B hold 1.0 min, flow rate: 25 mL/min) to provide to give the desired target.
^{t}: Additional Borylation step: A reaction mixture of bromo-substituted heterocycle (1.0 equivalents), bis(pinacolato)diborane (1.6 equivalents), Pd(dppf)Cl₂ (0.1 equivalent) and potassium acetate (3.0 equivalents) in dioxane (0.16 M) was degassed with nitrogen gas for 10 min then stirred at 80° C for 17 h. The suspension was concentrated in vacuo then purified by column chromatography (silica gel, 0-100% EtOAc: heptane then 0-10% MeOH: EtOAc) to provide to give the desired target.
^{u}: Additional Palladium coupling: To a reaction mixture of Na₂CO₃ (3.00 equivalent), borylate-substituted heterocycle (1.00 equivalents) and Pd(dppf)Cl₂ (0.10 equivalents) in dioxane (0.07 M) and H₂O (0.5 M) was added bromo-substituted heterocycle (1.15 equivalents). The suspension was degassed with nitrogen gas for a few minutes then stirred at 80° C for ~24 h and filtered over celite. The filtrate was concentrated in vacuo and purified by reverse phase HPLC (Sunfire C₁₈ 100 mm × 19 mm × 5 μm column, Mobile Phase A: H₂O/Mobile Phase B: ACN (0.05% TFA), 35 to 55% of Mobile Phase B over 10.0 min, 95% of Mobile Phase B hold 1.0 min, flow rate: 25 mL/min) to provide to give the desired target.
^{v}: Additional Palladium coupling: To a reaction mixture of Na₂CO₃ (3.00 equivalent), borylate-substituted heterocycle (1.00 equivalents) and Pd(dppf)Cl₂ (0.10 equivalents) in dioxane (0.07 M) and H₂O (0.5 M) was added bromo-substituted heterocycle (1.15 equivalents). The suspension was degassed with nitrogen gas for a few minutes then stirred at 80° C for ~24 h and filtered over celite. The filtrate was concentrated in vacuo and purified by reverse phase HPLC (Sunfire C₁₈ 100 mm × 19 mm × 5 μm column, Mobile Phase A: H₂O/Mobile Phase B: ACN (0.05% TFA), 35 to 55% of Mobile Phase B over 10.0 min, 95% of Mobile Phase B hold 1.0 min, flow rate: 25 mL/min) to provide to give the desired target.
^{w}: SNAr altered: To a reaction mixture of amine (1.0 equivalent) and fluoro and borylate-substituted heterocycle (2.0 equivalents) in ACN (0.1 M) and DMSO (1.0 M) was added TEA (10.0 equivalents). The reaction was stirred at 80º C for 48 h then the crude mixture was used directly in the next step.
^{x}: Additional Palladium coupling: A reaction mixture of K₂CO₃ (5.00 equivalent), boronic acid-substituted heterocycle (1.50 equivalents), Pd(dppf)Cl₂ (0.10 equivalent) and APhos Pd G3 (0.10 equivalents) was degassed with nitrogen gas then suspended in dioxane (0.04 M) and H₂O (0.15 M). The reaction mixture was stirred at 70° C for 1 h and 30 min then concentrated in vacuo. The residue was dissolved in DMSO, filtered and purified by reverse phase HPLC (Sunfire C₁₈ 100 mm × 19 mm × 5 μm column, Mobile Phase A: H₂O/Mobile Phase B: ACN (0.05% TFA), 60 to 95% of Mobile Phase B over 10.0 min, 95% of Mobile Phase B hold 1.0 min, flow rate: 25 mL/min) to provide to give the desired target.
^{y}: Additional Palladium coupling: A reaction mixture of K₂CO₃ (4.00 equivalent), boronic acid-substituted heterocycle (1.00 equivalents), N-XantPhos Pd G3 (0.10 equivalent) and bromo-substituted heterocycle (1.50 equivalents) was degassed with nitrogen gas then suspended in dioxane (0.04 M) and H₂O (0.15 M). The reaction mixture was stirred at room temperature for 72 h then stirred to 50° C for 2 h. To the suspension was added an additional portion of N-XantPhos Pd G3 (0.10 equivalent) then stirred to 50° C for 2 h. The reaction mixture was stirred at 90° C for 1 h then concentrated in vacuo to provide to give the desired target.

### Chiral Purification Data:

Example 50\hat{ }: The chiral purity was determined using SFC analytical method on Chiralpak AD-3, 50 mm×4.6 mm×3 μm column; Mobile phase A: CO₂; Mobile phase B: EtOH with 0.2% of NH₃; isocratic 40% of Mobile Phase B; flow rate: 3.5 mL/min; column temperature: 35 °C, backpressure: 1500 psi to provide Example 50 ((SFC-MS) m/z (M+H)⁺=519.37 at retention time: 2.047 min, 100% ee).

Example 51\hat{ }: A crude mixture was separated into its component isomers using SFC purification [Column: DAICEL CHIRALCEL OD 250 mm×30 mm×10 μm; Mobile phase: 45% CO₂/55% EtOH (0.1% NH₄OH); flow rate: 80 mL/min; gradient time=45 min]. The chiral purity was determined using SFC analytical method on Chiralcel OJ-3 50 mm×4.6 mm×3 μm column; Mobile phase A: CO₂; Mobile phase B: EtOH with 0.2% of NH₃; gradient: 5-40% of Mobile Phase B in 1.5 min and hold 40% for 1.0 min then 5% of Mobile Phase B for 0.5 min; flow rate: 3.0 mL/min; column temperature: 35 °C, backpressure: 1500 psi to provide Example 51 ((SFC-MS) m/z (M+H)⁺=519.46 at retention time: 1.735 min, 100% ee).

Example 53 and 54\hat{ }: A mixture of Example 53 and Example 54 were separated into its component isomers using SFC [Column: DAICEL CHIRALCEL OD 250 mm×30 mm×10 μm; Mobile phase: 40% CO₂/60% isopropyl alcohol with 0.1% of NH₄OH; flow rate: 80 mL/min; gradient time: 45 min]. The chiral purity was determined using SFC analytical method on Chiralcel OD-3 50 mm×4.6 mm×3 μm column; Mobile phase A: CO₂; Mobile phase B: isopropanol with 0.05% of DEA; isocratic: 40% of Mobile Phase B; flow rate: 4.0 mL/min; backpressure: 1500 psi; column temperature: 35 °C to provide Example 54: peak 1 ((SFC-MS) m/z (M+H)⁺=519.30 at retention time: 2.019 min, 100% ee) and Example 53: peak 2 ((SFC-MS) m/z (M+H)⁺=519.28 at retention time: 3.607 min, 100% ee).

Example 58 and 78\hat{ }: Example 77 was separated into its component isomers using SFC [Column: DAICEL CHIRALCEL OD 250 mm×30 mm×10 μm; Mobile phase: 40% CO₂/60% MeOH (0.1% NH₄OH); flow rate: 80 mL/min; gradient time=45 min]. The chiral purity was determined using SFC analytical method on Chiralcel OD-3 50 mm×4.6 mm×3 μm column; Mobile phase A: CO₂; Mobile phase B: MeOH with 0.2% of NH₃; isocratic: 40% of Mobile Phase B; flow rate: 3.5 mL/min; backpressure: 1500 psi; column temperature: 35 °C to provide Example 58: peak 1 ((SFC-MS) m/z (M+H)⁺=533.41 at retention time: 0.778 min, 100% ee) and Example 78: peak 2 ((SFC-MS) m/z (M+H)⁺=533.41 at retention time: 1.392 min, 100% ee).

Example 63\hat{ }: A mixture was separated into its component isomers using SFC [Column: DAICEL CHIRALCEL OD 250 mm×30 mm×10 μm; Mobile phase: 40% CO₂/60% EtOH with 0.1% of NH₄OH; flow rate: 60 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.05% of DEA; isocratic: 40% of Mobile Phase B; flow rate: 4.0 mL/min; backpressure: 1500 psi; column temperature: 35 °C to provide Example 63: peak 1 (retention time: 0.710 min, 100% ee) and peak 2 (retention time: 1.175 min, 100% ee).

Example 82 and 83\hat{ }: A crude material was purified using SFC [Column: DAICEL CHIRALPAK AD 250 mm×30 mm×10 μm; Mobile phase: 45% CO₂/55% isopropyl alcohol (0.1% NH₄OH); flow rate=80 mL/min]. The chiral purity was determined using SFC analytical method on Chiralpak AD-3 50 mm×4.6 mm×3 μm column; Mobile phase A: CO₂; Mobile phase B: isopropyl alcohol with 0.05% of DEA; isocratic: 40% of Mobile Phase B; flow rate: 4.0 mL/min; backpressure: 1500 psi; column temperature: 35 °C to provide Example 82: peak 1 (retention time: 1.233 min, 100% ee) and Example 83: peak 2 (retention time: 1.622 min, 97% ee).

Example 87 and 237\hat{ }: Example 62 was separated into its component isomers using SFC [Column: DAICEL CHIRALPAK AD 250 mm×30 mm×10 μm; Mobile phase: 50% CO₂/50% isopropyl alcohol (0.1% NH₄OH); flow rate=150 mL/min]. The chiral purity was determined using SFC analytical method on Chiralcel OD-3, 50 mm×4.6 mm×3 μm; Mobile phase A: CO₂; Mobile phase B: EtOH with 0.2% NH₃; isocratic gradient: 40% of Mobile Phase B; flow rate: 3.5 mL/min; column temperature: 35 °C, backpressure: 1500 psi to provide Example 237: peak 1 ((SFC-MS) retention time: 2.293 min, 100% ee) and Example 87: peak 2 ((SFC-MS) retention time: 3.434 min, 100% ee)

Example 111\hat{ }: A crude material was purified using SFC [Column: DAICEL CHIRALCEL OD 250 mm×30 mm×10 μm; Mobile phase: 45% CO₂/55% EtOH (0.1% NH₄OH); flow rate=80 mL/min; gradient time=45 min]. The chiral purity was determined using SFC analytical method on Chiralpak AD-3, 50 mm×4.6 mm×3 μm column; Mobile phase A: CO₂; Mobile phase B: isopropyl alcohol with 0.05% DEA; isocratic gradient: 40% of Mobile Phase B; flow rate: 4.0 mL/min; column temperature: 35 °C, backpressure: 1500 psi to provide Example 111: peak 1 ((SFC-MS) retention time: 4.224 min, 91% ee).

Example 112 and 128\hat{ }: Example 95 was separated into its component isomers using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.2% NH₃; isocratic gradient: 40% of Mobile Phase B; flow rate: 3.5 mL/min; column temperature: 35 °C, backpressure: 1500 psi to provide Example 112: peak 1 ((SFC-MS) m/z (M+H)⁺=547.43 at retention time: 0.764 min, 100% ee) and Example 128: peak 2 ((SFC-MS) m/z (M+H)⁺=547.44 at retention time: 1.380 min, 100% ee).

Example 117 and 118\hat{ }: Example 116 was separated into its component isomers using SFC [Column: Chiral Technologies OJ-H 250 mm×21 mm×5 μm; Mobile phase A: 85% CO₂; Mobile Phase B: 15% MeOH (0.2% NH₄OH); isocratic: 15% of Mobile Phase B; flow rate=75 mL/min, gradient time=20 min, backpressure=120 bar]. The chiral purity was determined using SFC analytical method on Chiral Technologies OJ-H 100 mm×4.6 mm×5 μm column; Mobile phase A: 75% CO₂, Mobile phase B: 25% MeOH with 0.2% of NH₄OH; isocratic: 25% of Mobile Phase B; flow rate: 5.0 mL/min; backpressure: 120 bar, gradient time=5 min to provide Example 117: peak 1 ((SFC-MS) m/z (M+H)⁺=547.3 at retention time: 2.61 min) and Example 118: peak 2 ((SFC-MS) m/z (M+H)⁺=547.3 at retention time: 2.98 min).

Example 119 and 557\hat{ }: A crude material was purified using SFC [Column: DAICEL CHIRALCEL OD 250 mm×30 mm×10 μm; Mobile phase: 40% CO₂/60% EtOH with 0.1% of NH₄OH; flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.05% of DEA; isocratic: 40% of Mobile Phase B; flow rate: 4.0 mL/min; backpressure: 1500 psi; column temperature: 35 °C to provide Example 557: peak 1 (retention time: 0.784 min, 100% ee) and Example 119: peak 2 (retention time: 1.425 min, 100% ee).

Example 122\hat{ }: A crude material was purified using SFC [Column: DAICEL CHIRALCEL OJ 250 mm×30 mm×10 μm; Mobile phase: 65% CO₂/35% EtOH (0.1% NH₄OH); flow rate=80 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 50% of Mobile Phase B; flow rate: 3.5 mL/min; column temperature: 35 °C, backpressure: 1500 psi to provide Example 122: peak 1 ((SFC-MS) m/z (M+H)⁺=528.44 at retention time: 1.045 min, 100% ee).

Example 123 and 124\hat{ }: A crude material was separated into its component isomers using SFC [Column: Chiral Technologies OJ-H 250 mm×21 mm×5 μm; Mobile phase A: 80% CO₂; Mobile Phase B: 20% MeOH with 0.2% of NH₄OH; isocratic: 20% of Mobile Phase B; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: 80% CO₂, Mobile phase B: 20% MeOH with 0.2% of NH₄OH; isocratic: 20% of Mobile Phase B; flow rate: 5.0 mL/min; backpressure: 120 bar, gradient time=5 min to provide Example 123: peak 1 ((SFC-MS) m/z (M+H)⁺=581.3 at retention time: 2.37 min) and Example 124: peak 2 ((SFC-MS) m/z (M+H)⁺=581.3 at retention time: 4.00 min).

Example 127 and 552\hat{ }: Example 532 was purified using SFC [Column: DAICEL CHIRALPAK AD 250 mm×30 mm×10 μm; Mobile phase: 50% CO₂ with ACN/50% MeOH with 0.1% NH₄OH; flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35 °C, backpressure: 1500 psi to provide Example 127: peak 1 ((SFC-MS) m/z (M+H)⁺=548.51 at retention time: 1.085 min, 100% ee) and Example 552: peak 2 ((SFC-MS) m/z (M+H)⁺=548.51 at retention time: 1.489 min, 100% ee).

Example 131 and 336\hat{ }: Example 130 was purified using SFC [Column: DAICEL CHIRALCEL OD 250 mm×30 mm×10 μm; Mobile phase: 40% CO₂/60% EtOH with 0.1% of NH₄OH; flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 3.5 mL/min; backpressure: 1500 psi; column temperature: 35 °C to provide Example 131: peak 1 ((SFC-MS) m/z (M+H)⁺=533.44 at retention time: 0.973 min, 99% ee) and Example 336: peak 2 ((SFC-MS) m/z (M+H)⁺=533.45 at retention time: 1.841 min, 100% ee).

Example 190 and 191\hat{ }: Example 90 was separated into its component isomers using SFC [Column: DAICEL CHIRALPAK IG 250 mm×30 mm×10 μm; Mobile phase: 80% MeOH/20% ACN; flow rate=100 mL/min]. The chiral purity was determined using SFC analytical method on Chiralpak IG-3 50 mm×4.6 mm×3 μm column; Mobile phase A: MeOH (0.05% of DEA); Mobile phase B: ACN; isocratic gradient: 20% of Mobile Phase B; flow rate: 1.0 mL/min; column temperature: 35 °C to provide Example 190: peak 1 ((SFC-MS) retention time: 3.431 min, 100% ee) and Example 191: peak 2 ((SFC-MS) retention time: 4.646 min, 100% ee).

Example 203 and 204\hat{ }: Example 59 was separated into its component isomers using SFC [Column: DAICEL CHIRALCEL OD 250 mm×30 mm×10 μm; Mobile phase: 45% CO₂/55% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.05% of DEA); gradient: 5-40% of Mobile Phase B in 1.5 min and hold at 40% of Mobile Phase B for 1.0 min then 5% of Mobile phase B for 0.5 min; flow rate: 4.0 mL/min; column temperature: 35 °C, backpressure: 1500 psi to provide Example 204: peak 1 ((SFC-MS) m/z (M+H)+=528.17 at retention time: 2.106 min, 100% ee) and Example 203: peak 2 ((SFC-MS) m/z (M+H)+=528.17 at retention time: 2.407 min, 99% ee).

Example 230 and 231\hat{ }: A mixture was separated into its component isomers using SFC [Column: Lux Cellulose 1 250 mm×21 mm×5 μm; Mobile phase: 35% MeOH (0.2% NH₄OH)/65% ACN; flow rate=75 mL/min, gradient time=20 min, backpressure=120 bar]. The chiral purity was determined using SFC analytical method on Lux Cellulose 1 100 mm×4.6 mm×5 μm column; Mobile phase A: MeOH (0.2% NH₄OH); Mobile phase B: ACN; isocratic gradient: 50% of Mobile Phase B; flow rate: 5.0 mL/min; backpressure: 120 bar, gradient time: 5 min to provide Example 230: peak 1 ((SFC-MS) m/z (M+H)⁺=545.2 at retention time: 2.92 min) and Example 231: peak 2 ((SFC-MS) m/z (M+H)⁺=545.3 at retention time: 3.87 min).

Example 232 and 233\hat{ }: Example 205 was separated into its component isomers using SFC [Column: DAICEL CHIRALCEL OD 250 mm×30 mm×10 μm; Mobile phase: 40% CO₂/60% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.05% of DEA); isocratic gradient: 40% of Mobile Phase B; flow rate: 4.0 mL/min; column temperature: 35 °C, backpressure: 1500 psi to provide Example 233: peak 1 ((SFC-MS) retention time: 0.883 min, 100% ee) and Example 232: peak 2 ((SFC-MS) retention time: 1.583 min, 100% ee).

Example 234 and 235\hat{ }: Example 85 was separated into its component isomers using SFC [Column: Chiral Technologies AD-H 250 mm×30 mm×5 μm; Mobile phase: 55% CO₂/45% 2-propanol (0.2% isopropylamine); flow rate=80 mL/min, backpressure=120 bar, temperature=40 °C]. The chiral purity was determined using SFC analytical method on Chiral Technologies AD-H 250 mm×4.6 mm×5 μm column; Mobile phase A: CO₂; Mobile phase B: 2-propanol (0.2% isopropylamine); gradient: 5-60% of Mobile Phase B over 8.0 min then 5% of Mobile Phase B over 0.50 min; flow rate: 3.0 mL/min; column temperature: 40 °C, backpressure: 120 bar to provide Example 234: peak 1 ((SFC-MS) retention time: 7.391 min, 100% ee) and Example 235: peak 2 ((SFC-MS) retention time: 7.794 min, 97% ee).

Example 250 and 251\hat{ }: A mixture was separated into its component isomers using SFC [Column: Lux Cellulose 3 250 mm×21 mm×5 μm; Mobile phase: 20% MeOH (0.2% NH₄OH)/80% ACN; flow rate=75 mL/min, gradient time=10 min, backpressure=120 bar]. The chiral purity was determined using SFC analytical method on Lux Cellulose 3 100 mm×4.6 mm×5 μm column; Mobile phase A: MeOH (0.2% NH₄OH); Mobile phase B: ACN; isocratic gradient: 75% of Mobile Phase B; flow rate: 5.0 mL/min; backpressure: 120 bar, gradient time: 5 min to provide Example 250: peak 1 ((SFC-MS) m/z (M+H)⁺=587.2 at retention time: 2.12 min) and Example 251: peak 2 ((SFC-MS) m/z (M+H)⁺=587.2 at retention time: 2.97 min).

Example 258 and 556\hat{ }: A crude material was purified using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH (0.05% DEA); isocratic gradient: 40% of Mobile Phase B; flow rate: 4.0 mL/min; column temperature: 35 °C, backpressure: 1500 psi to provide Example 556: peak 1 ((SFC-MS) m/z (M+H)+=589.23 at retention time: 0.998 min, 100% ee) and Example 258: peak 2 ((SFC-MS) m/z (M+H)+=589.19 at retention time: 1.724 min, 100% ee).

Example 262 and 553\hat{ }: A crude material was purified using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min]. The chiral purity was determined using SFC analytical method on Chiralcel OD-3 50 mm×4.6 mm×3 μm column; Mobile phase A: CO₂; Mobile phase B: EtOH (0.05% DEA); isocratic gradient: 40% of Mobile Phase B; flow rate: 4.0 mL/min; column temperature: 35 °C, backpressure: 1500 psi to provide Example 553: peak 1 ((SFC-MS) retention time: 0.487 min, 100% ee) and Example 262: peak 2 ((SFC-MS) retention time: 0.799 min, 100% ee).

Example 267 and 268\hat{ }: Example 249 was separated into its component isomers using SFC [Column: Pirkle Covalent (R,R) Whelk-01 250 mm×21.1 mm×5 μm; Mobile phase: 40% MeOH (0.2% NH₄OH)/60% ACN; flow rate=75 mL/min, gradient time=40 min, backpressure=120 bar]. The chiral purity was determined using SFC analytical method on Regis (R,R) Whelk-01 50 mm×5 mm×3 μm column; Mobile phase A: MeOH (0.2% NH₄OH); Mobile phase B: ACN; isocratic gradient: 55% of Mobile Phase B; flow rate: 5.0 mL/min; backpressure: 120 bar, gradient time: 10 min to provide Example 267: peak 1 ((SFC-MS) m/z (M+H)⁺=561.2 at retention time: 3.23 min) and Example 268: peak 2 ((SFC-MS) m/z (M+H)⁺=561.2 at retention time: 2.72 min).

Example 269 and 270\hat{ }: Example 260 was purified using SFC [Column: DAICEL CHIRALPAK IF 250 mm×30 mm×10 μm; Mobile phase: 30% heptane/70% (4:1, isopropyl alcohol: ACN) with 0.1% NH₄OH; flow rate=80 mL/min]. The chiral purity was determined using SFC analytical method on Chiralpak IF 100 mm×4.6 mm×3 μm column; Mobile phase A: hexane (0.1% DEA); Mobile phase B: (2:1, isopropyl alcohol: ACN); isocratic gradient: 60% of Mobile Phase B; flow rate: 1.0 mL/min; column temperature: 35 °C to provide Example 269: peak 1 ((SFC-MS) retention time: 3.315 min, 100% ee) and Example 270: peak 2 ((SFC-MS) retention time: 6.412 min, 100% ee)

Example 271 and 272\hat{ }: Example 265 was purified using SFC [Column: DAICEL CHIRALCEL OD 250 mm×30 mm×10 μm; Mobile phase: 40% CO₂/60% EtOH (0.1% NH₄OH); flow rate=80 mL/min]. The chiral purity was determined using SFC analytical method on Chiralpak AD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35 °C, backpressure: 1500 psi to provide Example 271: peak 1 ((SFC-MS) m/z (M+H)⁺=547.21 at retention time: 1.545 min, 100% ee) and Example 272: peak 2 ((SFC-MS) m/z (M+H)⁺=547.21 at retention time: 3.340 min, 100% ee).

Example 273\hat{ }: A crude material was purified using SFC [Column: DAICEL CHIRALPAK AD 250 mm×30 mm×10 μm; Mobile phase: 50% CO₂/50% isopropyl alcohol (0.1% NH₄OH); flow rate=150 mL/min; gradient time=4 min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35 °C, backpressure: 1500 psi to provide Example 273: peak 1 ((SFC-MS) m/z (M+H)+=520.15 at retention time: 1.272 min, 100% ee).

Example 274 and 275\hat{ }: Example 68 was purified using SFC [Column: CHIRALPAK IH 250 mm×30 mm×10 μm; Mobile phase: 45% CO₂/55% EtOH (0.1% NH₄OH); flow rate=140 mL/min, gradient time=4 min]. The chiral purity was determined using SFC analytical method on Chiralpak AS-3 50 mm×4.6 mm×3 μm column; Mobile phase A: CO₂; Mobile phase B: EtOH (0.2% NH₃); gradient: 5-40% of Mobile Phase B in 2.0 min then hold for 40% of Mobile Phase B in 1.2 min then 5% of Mobile Phase B for 0.8 min; flow rate: 3.0 mL/min; column temperature: 35 °C, backpressure: 1500 psi to provide Example 275: peak 1 ((SFC-MS) m/z (M+H)⁺=545.16 at retention time: 2.593 min, 99% ee) and Example 274: peak 2 ((SFC-MS) m/z (M+H)⁺=545.17 at retention time: 3.311 min, 100% ee).

Example 276 and 277\hat{ }: Example 255 was separated into its component isomers using SFC [Column: Lux Cellulose 3 250 mm×21 mm×5 μm; Mobile phase: 15% MeOH (0.2% NH₄OH)/85% ACN; flow rate=75 mL/min, gradient time=20 min, backpressure=120 bar]. The chiral purity was determined using SFC analytical method on Lux Cellulose 3 100 mm×4.6 mm×5 μm column; Mobile phase A: MeOH (0.2% NH₄OH); Mobile phase B: ACN; isocratic gradient: 80% of Mobile Phase B; flow rate: 5.0 mL/min; backpressure: 120 bar, gradient time: 5 min to provide Example 276: peak 1 ((SFC-MS) m/z (M+H)⁺=601.2 at retention time: 2.90 min) and Example 277: peak 2 ((SFC-MS) m/z (M+H)⁺=601.2 at retention time: 3.90 min).

Example 280 and 281\hat{ }: A mixture was separated into its component isomers using SFC [Column: Lux Cellulose 1 250 mm×21 mm×5 μm; Mobile phase: 40% MeOH (0.2% NH₄OH)/60% ACN; flow rate=75 mL/min, gradient time=40 min, backpressure=120 bar]. The chiral purity was determined using SFC analytical method on Lux Cellulose 1 100 mm×4.6 mm×5 μm column; Mobile phase A: MeOH (0.2% NH₄OH); Mobile phase B: ACN; isocratic gradient: 40% of Mobile Phase B; flow rate: 5.0 mL/min; backpressure: 120 bar, gradient time: 10 min to provide Example 280: peak 1 ((SFC-MS) m/z (M+H)⁺=531.2 at retention time: 4.84 min) and Example 281: peak 2 ((SFC-MS) m/z (M+H)⁺=531.2 at retention time: 6.81 min).

Example 285 and 551\hat{ }: A mixture was purified using SFC [Column: YMC-IB 250 mm×30 mm×10 μm; Mobile phase: 70% heptane/30% (4:1, isopropyl alcohol: ACN) with 0.1% NH₄OH; flow rate=100 mL/min]. The chiral purity was determined using SFC analytical method on Chiralpak IB-N 100 mm×4.6 mm×3 μm column; Mobile phase A: hexane (0.1% DEA); Mobile phase B: (2:1, isopropyl alcohol: ACN); isocratic gradient: 40% of Mobile Phase B; flow rate: 1.0 mL/min; column temperature: 35 °C to provide Example 551: peak 1 ((SFC-MS) retention time: 4.066 min, 100% ee) and Example 285: peak 2 ((SFC-MS) retention time: 4.601 min, 99% ee).

Example 287\hat{ }: A crude material was purified using SFC [Column: DAICEL CHIRALCEL OD 250 mm×30 mm×10 μm; Mobile phase: 40% CO₂/60% EtOH (0.1% NH₄OH); flow rate=80 mL/min; gradient time=45 min] to provide Example 287. The chiral purity was determined using SFC analytical method on Chiralcel OD-3 50 mm×4.6 mm×3 μm column; Mobile phase A: CO₂; Mobile phase B: EtOH with 0.2% of NH₃; gradient: 5-40% of Mobile Phase B over 2.0 min and held at 40% of Mobile Phase B for 1.2 min then % of Mobile Phase B for 0.8 min; flow rate: 4.0 mL/min; backpressure: 1500 psi; column temperature: 35 °C to provide Example 287: peak 1 ((SFC-MS) m/z (M+H)⁺=559.20 at retention time: 2.903 min, 100% ee).

Example 303 and 304\hat{ }: Example 282 was separated into its component isomers using SFC [Column: Chiral Technologies OJ-H 250 mm×21 mm×5 μm; Mobile phase: 25% MeOH (0.2% NH₄OH)/75% ACN; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: MeOH (0.2% NH₄OH); Mobile phase B: ACN; isocratic gradient: 70% of Mobile Phase B; flow rate: 5.0 mL/min; backpressure: 120 bar, gradient time: 5 min to provide Example 303: peak 1 ((SFC-MS) m/z (M+H)⁺=587.2 at retention time: 1.92 min) and Example 304: peak 2 ((SFC-MS) m/z (M+H)⁺=587.2 at retention time: 2.74 min).

Example 307 and 550\hat{ }: A mixture was purified using SFC [Column: DAICEL CHIRALCEL OD 250 mm×30 mm×10 μm; Mobile phase: 50% CO₂/50% MeOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: MeOH (0.2% NH₃); gradient: 5-40% of Mobile Phase B in 1.5 min and hold at 40% of Mobile Phase B for 1.0 min then 5% of Mobile phase B for 0.5 min; flow rate: 4.0 mL/min; column temperature: 35 °C, backpressure: 1500 psi to provide Example 550: peak 1 ((SFC-MS) m/z (M+H)⁺=587.19 at retention time: 1.669 min, 100% ee) and Example 307: peak 2 ((SFC-MS) m/z (M+H)⁺=587.16 at retention time: 1.945 min, 100% ee).

Example 310 and 311\hat{ }: Example 246 was separated into its component isomers using SFC [Column: Chiral Technologies IB 250 mm×21 mm×5 μm; Mobile phase: 40% MeOH (0.2% NH₄OH)/60% ACN; flow rate=75 mL/min, gradient time=10 min, backpressure=120 bar]. The chiral purity was determined using SFC analytical method on Chiral Technologies IB 100 mm×4.6 mm×5 μm column; Mobile phase A: MeOH (0.2% NH₄OH); Mobile phase B: ACN; isocratic gradient: 50% of Mobile Phase B; flow rate: 5.0 mL/min; backpressure: 120 bar, gradient time: 5 min to provide Example 310: peak 1 ((SFC-MS) m/z (M+H)⁺=561.3 at retention time: 2.14 min) and Example 311: peak 2 ((SFC-MS) m/z (M+H)⁺=561.3 at retention time: 3.12 min).

Example 312 and 313\hat{ }: Example 115 was separated into its component isomers using SFC [Column: Chiral Technologies IB 250 mm×21 mm×5 μm; Mobile phase: 40% MeOH (0.2% NH₄OH)/60% ACN; flow rate=75 mL/min, gradient time=10 min, backpressure=120 bar]. The chiral purity was determined using SFC analytical method on Chiral Technologies IB 100 mm×4.6 mm×5 μm column; Mobile phase A: MeOH (0.2% NH₄OH); Mobile phase B: ACN; isocratic gradient: 50% of Mobile Phase B; flow rate: 5.0 mL/min; backpressure: 120 bar, gradient time: 5 min to provide Example 312: peak 1 ((SFC-MS) m/z (M+H)⁺=547.3 at retention time: 2.40 min) and Example 313: peak 2 ((SFC-MS) m/z (M+H)⁺=547.3 at retention time: 3.61 min).

Example 316 and 317\hat{ }: Example 314 was separated into its component isomers using SFC [Column: Lux Cellulose 1 250 mm×21 mm×5 μm; Mobile phase: 35% MeOH (0.2% NH₄OH)/65% ACN; flow rate=75 mL/min, gradient time=10 min, backpressure=120 bar]. The chiral purity was determined using SFC analytical method on Lux Cellulose 1 100 mm×4.6 mm×5 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 5 min to provide Example 316: peak 2 ((SFC-MS) m/z (M+H)⁺=547.2 at retention time: 2.40 min) and Example 317: peak 3 ((SFC-MS) m/z (M+H)⁺=547.2 at retention time: 2.94 min).

Example 318 and 319\hat{ }: A crude material was purified using SFC [Column: DAICEL CHIRALPAK AD 250 mm×30 mm×10 μm; Mobile phase: 50% CO₂/50% isopropyl alcohol (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35 °C, backpressure: 1500 psi to provide Example 318: peak 1 ((SFC-MS) m/z (M+H)⁺=561.20 at retention time: 1.188 min, 100% ee) and Example 319: peak 2 ((SFC-MS) m/z (M+H)⁺=561.22 at retention time: 1.900 min, 99% ee).

Example 335 and 549\hat{ }: A crude material was purified using SFC [Column: DAICEL CHIRALPAK AD 250 mm×30 mm×10 μm; Mobile phase: 50% CO₂/50% EtOH (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35 °C, backpressure: 1500 psi to provide Example 549: peak 1 ((SFC-MS) m/z (M+H)⁺=528.18 at retention time: 0.811 min, 99% ee) and Example 335: peak 2 ((SFC-MS) m/z (M+H)⁺=528.25 at retention time: 1.497 min, 100% ee).

Example 347 and 348\hat{ }: Example 358 was purified using SFC [Column: DAICEL CHIRALPAK AD 250 mm×30 mm×10 μm; Mobile phase: 50% CO₂/50% isopropyl alcohol (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 4.0 mL/min; column temperature: 35 °C, backpressure: 1500 psi to provide Example 347: peak 1 ((SFC-MS) m/z (M+H)⁺=558.17 at retention time: 1.067 min, 100% ee) and Example 348: peak 2 ((SFC-MS) m/z (M+H)⁺=558.21 at retention time: 1.503 min, 98% ee).

Example 352 and 353\hat{ }: The crude mixture was separated into its component isomers using SFC [Column: Chiral Technologies IH 250 mm×21 mm×5 μm; Mobile phase: 35% MeOH (0.2% NH₄OH)/65% ACN; flow rate=75 mL/min, gradient time=10 min, backpressure=120 bar]. The chiral purity was determined using SFC analytical method on Chiral Technologies IH 100 mm×4.6 mm×5 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 5 min to provide Example 352: peak 1 ((SFC-MS) m/z (M+H)⁺=547.2 at retention time: 1.97 min) and Example 353: peak 2 ((SFC-MS) m/z (M+H)⁺=547.3 at retention time: 3.05 min).

Example 354 and 355\hat{ }: Example 315 was separated into its component isomers using SFC [Column: Chiral Technologies OD-H 250 mm×21 mm×5 μm; Mobile phase: 25% MeOH (0.2% NH₄OH)/75% ACN; flow rate=75 mL/min, gradient time=20 min, backpressure=120 bar]. The chiral purity was determined using SFC analytical method on Chiral Technologies OD-H 100 mm×4.6 mm×5 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 5 min to provide Example 354: peak 1 ((SFC-MS) m/z (M+H)⁺=561.4 at retention time: 2.67 min) and Example 355: peak 2 ((SFC-MS) m/z (M+H)⁺=561.3 at retention time: 3.19 min).

Example 356 and 357\hat{ }: The crude mixture was separated into its component isomers using SFC [Column: Chiral Technologies OJ-H 250 mm×21 mm×5 μm; Mobile phase: 20% MeOH (0.2% NH₄OH)/80% ACN; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: MeOH (0.2% NH₄OH); Mobile phase B: ACN; isocratic gradient: 75% of Mobile Phase B; flow rate: 5.0 mL/min; backpressure: 120 bar, gradient time: 5 min to provide Example 356: peak 1 ((SFC-MS) m/z (M+H)⁺=572.2 at retention time: 1.78 min) and Example 357: peak 2 ((SFC-MS) m/z (M+H)⁺=572.2 at retention time: 3.51 min).

Example 359 and 360\hat{ }: Example 351 was purified using SFC [Column: DAICEL CHIRALPAK IF 250 mm×30 mm×10 μm; Mobile phase: 50% heptane/50% (4:1, isopropyl alcohol: ACN); flow rate=100 mL/min]. The chiral purity was determined using SFC analytical method on Chiralpak IF 100 mm×4.6 mm×3 μm column; Mobile phase A: hexane (0.1% DEA); Mobile phase B: (2:1, isopropyl alcohol: ACN); isocratic gradient: 60% of Mobile Phase B; flow rate: 1.0 mL/min; column temperature: 35 °C to provide Example 359: peak 1 ((SFC-MS) retention time: 3.568 min, 100% ee) and Example 360: peak 2 ((SFC-MS) retention time: 4.561 min, 98% ee).

Example 361 and 362\hat{ }: Example 322 was purified using SFC [Column: DAICEL CHIRALCEL OD 250 mm×30 mm×10 μm; Mobile phase: 45% CO₂/55% EtOH (0.1% NH₄OH); flow rate=80 mL/min]. The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 4.0 mL/min; backpressure: 1500 psi; column temperature: 35 °C to provide Example 361: peak 1 ((SFC-MS) m/z (M+H)⁺=532.18 at retention time: 0.818 min, 100% ee) and Example 362: peak 2 ((SFC-MS) m/z (M+H)⁺=532.21 at retention time: 1.180 min, 99% ee).

Example 391 and 392\hat{ }: The crude mixture was separated into its component isomers using SFC [Column: Chiral Technologies OJ-H 250 mm×21 mm×5 μm; Mobile phase: 30% MeOH (0.2% NH₄OH)/70% ACN; flow rate=75 mL/min, gradient time=10 min, backpressure=120 bar]. The chiral purity was determined using SFC analytical method on Chiral Technologies OJ-H 100 mm×4.6 mm×5 μm column; Mobile phase A: MeOH (0.2% NH₄OH); Mobile phase B: ACN; isocratic gradient: 65% of Mobile Phase B; flow rate: 5.0 mL/min; backpressure: 120 bar, gradient time: 5 min to provide Example 391: peak 1 ((SFC-MS) m/z (M+H)⁺=573.2 at retention time: 1.93 min) and Example 392: peak 2 ((SFC-MS) m/z (M+H)⁺=573.2 at retention time: 3.20 min).

Example 393 and 394\hat{ }: Example 339 was purified using SFC [Column: DAICEL CHIRALCEL OJ 250 mm×30 mm×10 μm; Mobile phase: 45% CO₂/55% EtOH (0.1% NH₄OH); flow rate=80 mL/min]. The chiral purity was determined using SFC analytical method on Chiralcel OJ-3 50 mm×4.6 mm×3 μm column; Mobile phase A: CO₂; Mobile phase B: EtOH (0.2% NH₃); isocratic gradient: 40% of Mobile Phase B; flow rate: 3.0 mL/min; column temperature: 35 °C, backpressure: 1500 psi to provide Example 393: peak 1 ((SFC-MS) m/z (M+H)⁺=546.64 at retention time: 0.895 min, 100% ee) and Example 394: peak 2 ((SFC-MS) m/z (M+H)⁺=546.63 at retention time: 1.939 min, 99% ee).

Example 428 and 429\hat{ }: Example 399 was purified using SFC [Column: DAICEL CHIRALCEL OD 250 mm×30 mm×10 μm; Mobile phase: 45% CO₂/55% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35 °C, backpressure: 1500 psi to provide Example 428: peak 1 ((SFC-MS) m/z (M+H)⁺=532.22 at retention time: 0.636 min, 100% ee) and Example 429: peak 2 ((SFC-MS) m/z (M+H)⁺=532.21 at retention time: 1.130 min, 100% ee).

Example 444 and 445\hat{ }: Example 55 was purified using SFC [Column: ChiralPak IH 250 mm×30 mm×10 μm; Mobile phase: 50% CO₂/50% EtOH (0.1% NH₄OH); flow rate=150 mL/min, gradient time=4 min]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 40% of Mobile Phase B; flow rate: 3.0 mL/min; column temperature: 35 °C, backpressure: 1500 psi to provide Example 444: peak 1 ((SFC-MS) m/z (M+H)⁺=519.62 at retention time: 1.191 min, 100% ee) and Example 445: peak 2 ((SFC-MS) m/z (M+H)⁺=519.62 at retention time: 1.728 min, 99% ee).

Example 446 and 447\hat{ }: The crude mixture was separated into its component isomers using SFC [Column: Chiral Technologies IA 250 mm×21 mm×5 μm; Mobile phase: 40% MeOH (0.2% NH₄OH)/60% ACN; flow rate=75 mL/min, gradient time=20 min, backpressure=120 bar]. The chiral purity was determined using SFC analytical method on Chiral Technologies IA 100 mm×4.6 mm×5 μm column; Mobile phase A: MeOH (0.2% NH₄OH); Mobile phase B: ACN; isocratic gradient: 45% of Mobile Phase B; flow rate: 5.0 mL/min; backpressure: 120 bar, gradient time: 10 min to provide Example 446: peak 1 ((SFC-MS) m/z (M+H)⁺=548.3 at retention time: 2.33 min) and Example 447: peak 2 ((SFC-MS) m/z (M+H)⁺=548.3 at retention time: 6.75 min).

Example 448 and 449\hat{ }: The crude mixture was separated into its component isomers using SFC [Column: Chiral Technologies IA 250 mm×21 mm×5 μm; Mobile phase: 35% MeOH (0.2% NH₄OH)/65% ACN; flow rate=75 mL/min, gradient time=20 min, backpressure=120 bar]. The chiral purity was determined using SFC analytical method on Chiral Technologies IA 100 mm×4.6 mm×5 μm column; Mobile phase A: MeOH (0.2% NH₄OH); Mobile phase B: ACN; isocratic gradient: 45% of Mobile Phase B; flow rate: 5.0 mL/min; backpressure: 120 bar, gradient time: 10 min to provide Example 448: peak 1 ((SFC-MS) m/z (M+H)⁺=562.3 at retention time: 1.96 min) and Example 449: peak 2 ((SFC-MS) m/z (M+H)⁺=562.3 at retention time: 4.38 min).

Example 461 and 462\hat{ }: Example 459 was separated into its component isomers using chiral HPLC purification [Column: C₁₈ 150 mm×40 mm; Mobile phase: H₂O (NH₄OH—NH₄HCO₃)/ACN; gradient=13-53% of Mobile Phase B; flow rate=60 mL/min, gradient time=9 min]. The chiral purity was determined using SFC analytical method on Chiralpak AD-3 50 mm×4.6 mm×3 μm column; Mobile phase A: CO₂; Mobile phase B: EtOH (0.2% NH₃); isocratic gradient: 40% of Mobile Phase B; flow rate: 3.5 mL/min; column temperature: 35 °C, backpressure: 1500 psi to provide Example 461: peak 1 ((SFC-MS) m/z (M+H)⁺=602.71 at retention time: 0.837 min, 100% ee) and Example 462: peak 2 ((SFC-MS) m/z (M+H)⁺=602.70 at retention time: 1.799 min, 100% ee).

Example 464 and 465\hat{ }: Example 460 was purified using SFC [Column: DAICEL CHIRALPAK AD 250 mm×30 mm×10 μm; Mobile phase: 50% CO₂/50% EtOH (0.1% NH₄OH); flow rate=150 mL/min]. The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic gradient: 40% of Mobile Phase B; flow rate: 3.5 mL/min; column temperature: 35 °C, backpressure: 1500 psi to provide Example 464: peak 1 ((SFC-MS) m/z (M+H)⁺=588.69 at retention time: 0.715 min, 100% ee) and Example 465: peak 2 ((SFC-MS) m/z (M+H)⁺=588.67 at retention time: 2.128 min, 98% ee).

Example 467 and 468\hat{ }: Example 463 was purified using SFC [Column: DAICEL CHIRALCEL OD 250 mm×30 mm×10 μm; Mobile phase: 45% CO₂/55% MeOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: MeOH (0.2% NH₃); isocratic gradient: 40% of Mobile Phase B; flow rate: 4.0 mL/min; column temperature: 35 °C, backpressure: 1500 psi to provide Example 467: peak 1 ((SFC-MS) m/z (M+H)⁺=558.47 at retention time: 0.408 min, 100% ee) and Example 468: peak 2 ((SFC-MS) m/z (M+H)⁺=558.48 at retention time: 0.707 min, 99% ee).

Example 474 and 475\hat{ }: Example 472 was purified using SFC [Column: DAICEL CHIRALPAK AD 250 mm×30 mm×10 μm; Mobile phase: 40% CO₂/60% MeOH (0.1% NH₄OH); flow rate=80 mL/min, gradient time=45 min]. The chiral purity was determined using SFC analytical method on Chiralpak AD-3 50 mm×4.6 mm×3 μm column; Mobile phase A: CO₂; Mobile phase B: MeOH (0.2% NH₃); isocratic gradient: 40% of Mobile Phase B; flow rate: 3.5 mL/min; column temperature: 35 °C, backpressure: 1500 psi to provide Example 474: peak 1 ((SFC-MS) m/z (M+H)⁺=549.68 at retention time: 0.836 min, 100% ee) and Example 475: peak 2 ((SFC-MS) m/z (M+H)⁺=549.68 at retention time: 2.047 min, 100% ee).

Example 493 and 494\hat{ }: The crude mixture was purified using SFC [Column: DAICEL CHIRALCEL OD 250 mm×30 mm×10 μm; Mobile phase: 40% CO₂/60% MeOH (0.1% NH₄OH); flow rate=70 mL/min, gradient time=45 min, column temperature=40 °C, backpressure=100 bar]. The chiral purity was determined using SFC analytical method on Chiralcel OD-3 50 mm×4.6 mm×3 μm column; Mobile phase A: CO₂; Mobile phase B: MeOH (0.2% NH₃); isocratic gradient: 40% of Mobile Phase B; flow rate: 3.5 mL/min; column temperature: 35 °C, backpressure: 1500 psi to provide Example 493: peak 1 ((SFC-MS) m/z (M+H)⁺=583.71 at retention time: 0.784 min, 100% ee) and Example 494: peak 2 ((SFC-MS) m/z (M+H)⁺=583.69 at retention time: 1.855 min, 100% ee).

Example 506 and 507\hat{ }: The crude mixture was purified using SFC [Column: DAICEL CHIRALPAK AS 250 mm×30 mm×10 μm; Mobile phase: 50% CO₂/50% (4:1, EtOH: ACN) (0.1% NH₄OH); flow rate=150 mL/min, gradient time=40 min]. The chiral purity was determined using SFC analytical method on Chiralpak AS-3 100 mm×4.6 mm×3 μm column; Mobile phase A: CO₂; Mobile phase B: EtOH (0.2% NH₃); isocratic gradient: 40% of Mobile Phase B; flow rate: 2.8 mL/min; column temperature: 35 °C, backpressure: 1500 psi to provide Example 506: peak 1 ((SFC-MS) m/z (M+H)+=547.25 at retention time: 1.151 min, 100% ee) and Example 507: peak 2 ((SFC-MS) m/z (M+H)+=547.22 at retention time: 2.043 min, 100% ee).

Example 508 and 509\hat{ }: The crude mixture was purified using SFC [Column: DAICEL CHIRALPAK AS 250 mm×30 mm×10 μm; Mobile phase: 50% CO₂/50% (4:1, EtOH: ACN) (0.1% NH₄OH); flow rate=150 mL/min, gradient time=60 min]. The chiral purity was determined using SFC analytical method on Chiralpak AS-3 100 mm×4.6 mm×3 μm column; Mobile phase A: CO₂; Mobile phase B: EtOH (0.2% NH₃); isocratic gradient: 40% of Mobile Phase B; flow rate: 2.8 mL/min; column temperature: 35 °C, backpressure: 1500 psi to provide Example 508: peak 1 ((SFC-MS) m/z (M+H)+=548.26 at retention time: 1.285 min, 100% ee) and Example 509: peak 2 ((SFC-MS) m/z (M+H)+=548.31 at retention time: 3.523 min, 100% ee).

Example 510 and 511\hat{ }: The crude mixture was purified using SFC [Column: DAICEL CHIRALPAK AS 250 mm×30 mm×10 μm; Mobile phase: 45% CO₂/55% (1:1, EtOH: ACN) (0.1% NH₄OH); flow rate=140 mL/min, column temperature=40 °C, backpressure=100 bar]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 50% of Mobile Phase B; flow rate: 2.5 mL/min; column temperature: 35 °C, backpressure: 1500 psi to provide Example 510: peak 1 ((SFC-MS) m/z (M+H)⁺=534.55 at retention time: 0.702 min, 100% ee) and Example 511: peak 2 ((SFC-MS) m/z (M+H)⁺=534.58 at retention time: 1.561 min, 100% ee).

Example 514 and 515\hat{ }: The crude mixture was purified using SFC [Column: DAICEL CHIRALPAK AS 250 mm×30 mm×10 μm; Mobile phase: 45% CO₂/55% EtOH (0.1% NH₄OH); flow rate=140 mL/min, column temperature=40 °C, backpressure=100 bar]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 40% of Mobile Phase B; flow rate: 3.0 mL/min; column temperature: 35 °C, backpressure: 1500 psi to provide Example 514: peak 1 ((SFC-MS) m/z (M+H)⁺=533.56 at retention time: 0.979 min, 100% ee) and Example 515: peak 2 ((SFC-MS) m/z (M+H)⁺=533.56 at retention time: 1.597 min, 99% ee).

Example 527 and 528\hat{ }: A crude material was purified using SFC [Column: DAICEL CHIRALPAK AS 250 mm×30 mm×10 μm; Mobile phase: 45% CO₂/55% EtOH (0.1% NH₄OH); flow rate=140 mL/min, backpressure=100 bar, column temperature=40 °C]. The chiral purity was determined using SFC analytical method on Chiralpak AS-3 50 mm×4.6 mm×3 μm column; Mobile phase A: CO₂; Mobile phase B: EtOH (0.2% NH₃); isocratic gradient: 50% of Mobile Phase B; flow rate: 2.5 mL/min; column temperature: 35 °C, backpressure: 1500 psi to provide Example 527: peak 1 ((SFC-MS) m/z (M+H)⁺=561.59 at retention time: 0.509 min, 100% ee) and Example 528: peak 2 ((SFC-MS) m/z (M+H)⁺=561.59 at retention time: 1.377 min, 100% ee).

Example 529\hat{ }: Example 524 was separated into its component isomers using SFC [Column: Phenomenex Lux Cellulose 1 250 mm×21 mm×5 μm; Mobile phase: 30% MeOH/70% ACN; flow rate=75 mL/min, gradient time=20 min, backpressure=120 bar, column temperature=50 °C]. The chiral purity was determined using SFC analytical method on Lux Cellulose 1 100 mm×4.6 mm×5 μm column; Mobile phase A: MeOH; Mobile phase B: ACN; isocratic gradient: 60% of Mobile Phase B; flow rate: 5.0 mL/min; backpressure: 120 bar, gradient time: 5 min to provide peak 1 ((SFC-MS) m/z (M+H)⁺=561.2 at retention time: 3.28 min) and Example 529: peak 2 ((SFC-MS) m/z (M+H)+=561.2 at retention time: 3.40 min).

Example 538 and 539\hat{ }: Example 537 was purified using SFC [Column: DAICEL CHIRALCEL OD 250 mm×30 mm×10 μm; Mobile phase: 40% CO₂/60% EtOH (0.1% NH₄OH); flow rate=80 mL/min, gradient time=45 min]. The chiral purity was determined using SFC analytical method on Chiralcel OD-3 50 mm×4.6 mm×3 μm column; Mobile phase A: CO₂; Mobile phase B: EtOH (0.2% NH₃); isocratic gradient: 40% of Mobile Phase B; flow rate: 4.0 mL/min; column temperature: 35 °C, backpressure: 1500 psi to provide Example 539: peak 1 ((SFC-MS) m/z (M+H)⁺=546.24 at retention time: 0.607 min, 100% ee) and Example 538: peak 2 ((SFC-MS) m/z (M+H)⁺=546.22 at retention time: 1.034 min, 100% ee).

Example 547 and 548\hat{ }: Example 546 was separated into its component isomers using SFC [Column: Chiral Technologies OD-H 250 mm×21 mm×5 μm; Mobile phase: 40% CO₂/60% MeOH (0.2% NH₄OH); flow rate=75 mL/min, backpressure=120 bar, gradient time=10 min]. The chiral purity was determined using SFC analytical method on Chiral Technologies OD-H 100 mm×4.6 mm×5 μm column; Mobile phase A: CO₂; Mobile phase B: MeOH (0.2% NH₄OH); isocratic gradient: 50% of Mobile Phase B; flow rate: 5.0 mL/min; backpressure: 120 bar to provide Example 548: peak 1 ((SFC-MS) m/z (M+H)⁺=601.3 at retention time: 1.99 min). and Example 547: peak 2 ((SFC-MS) m/z (M+H)⁺=601.3 at retention time: 3.94 min).

Example 667 and 668\hat{ }: A mixture was separated into its component isomers using SFC [Column: Chiral Technologies AD-H 250 mm×30 mm×5 μm; Mobile phase: 60% CO₂/40% 2-propanol (0.2% isopropylamine); flow rate=100 mL/min, backpressure=100 bar, temperature=40 °C]. The chiral purity was determined using SFC analytical method on Chiral Technologies AD-H 250 mm×4.6 mm×5 μm column; Mobile phase A: CO₂; Mobile phase B: 2-propanol (0.2% isopropylamine); gradient: 5-100% of Mobile Phase B over 7.50 min then 5% of Mobile Phase B over 0.50 min; flow rate: 3.0 mL/min; column temperature: 40 °C, backpressure: 100 bar to provide Example 668: peak 1 ((SFC-MS) retention time: 6.547 min, 99% ee) and Example 667: peak 2 ((SFC-MS) retention time: 6.951 min, 96% ee).

Example 670 and 671\hat{ }: A mixture was separated into its component isomers using SFC [Column: Chiral Technologies AS-H 250 mm×21 mm×5 μm; Mobile phase A: 25% CO₂; Mobile Phase B: 75% MeOH (0.2% NH₄OH); isocratic: 75% of Mobile Phase B; flow rate=75 mL/min, gradient time=20 min, backpressure=120 bar]. The chiral purity was determined using SFC analytical method on Chiral Technologies AS-H 100 mm×4.6 mm×5 μm column; Mobile phase A: 25% CO₂, Mobile phase B: 75% MeOH with 0.2% of NH₄OH; isocratic: 75% of Mobile Phase B; flow rate: 5.0 mL/min; backpressure: 120 bar, gradient time=20 min to provide Example 671: peak 1 ((SFC-MS) m/z (M+H)⁺=645.4 at retention time: 8.71 min) and Example 670: peak 2 ((SFC-MS) m/z (M+H)+=645.3 at retention time: 13.48 min).

Example 672 and 673\hat{ }: Example 710 was separated into its component isomers using SFC [Column: Chiral Technologies IB 250 mm×21 mm×5 μm; Mobile phase A: 35% CO₂; Mobile Phase B: 65% MeOH (0.2% NH₄OH); isocratic: 65% of Mobile Phase B; flow rate=75 mL/min, gradient time=20 min, backpressure=120 bar]. The chiral purity was determined using SFC analytical method on Chiral Technologies IB 100 mm×4.6 mm×5 μm column; Mobile phase A: 40% CO₂, Mobile phase B: 60% MeOH with 0.2% of NH₄OH; isocratic: 60% of Mobile Phase B; flow rate: 5.0 mL/min; backpressure: 120 bar, gradient time=10 min to provide Example 673: peak 1 ((SFC-MS) m/z (M+H)⁺=631.3 at retention time: 5.53 min) and Example 672: peak 2 ((SFC-MS) m/z (M+H)+=631.3 at retention time: 6.86 min).

Example 692 and 693\hat{ }: Example 701 was separated into its component isomers using SFC [Column: DAICEL CHIRALCEL OD 250 mm×30 mm×10 μm; Mobile phase: 50% CO₂/50% EtOH (0.1% NH₄OH); isocratic: 50% of Mobile Phase B; flow rate: 80 mL/min; backpressure=100 bar, column temperature=40 °C]. The chiral purity was determined using SFC analytical method on Chiralcel OD-3 50 mm×4.6 mm×3 μm column; Mobile phase A: CO₂; Mobile phase B: EtOH with 0.2% of NH₃; gradient: 5-40% of Mobile Phase B in 2.0 min and hold 40% for 1.2 min then 5% of Mobile Phase B for 0.8 min; flow rate: 4.0 mL/min; backpressure: 1500 psi; column temperature: 35 °C to provide Example 693: peak 1 ((SFC-MS) m/z (M+H)⁺=656.34 at retention time: 2.201 min, 100% ee) and Example 692: peak 2 ((SFC-MS) m/z (M+H)⁺=656.35 at retention time: 2.705 min, 100% ee).

Example 694 and 695\hat{ }: Example 696 was separated into its component isomers using SFC [Column: DAICEL CHIRALCEL OD 250 mm×30 mm×10 μm; Mobile phase: 40% CO₂/60% EtOH (0.1% NH₄OH); isocratic: 60% of Mobile Phase B; flow rate: 80 mL/min; backpressure=100 bar, column temperature=40 °C]. The chiral purity was determined using SFC analytical method on Chiralcel OD-3 50 mm×4.6 mm×3 μm column; Mobile phase A: CO₂; Mobile phase B: EtOH with 0.2% of NH₃; isocratic: 40% of Mobile Phase B; flow rate: 3.5 mL/min; backpressure: 1500 psi; column temperature: 35 °C to provide Example 695: peak 1 ((SFC-MS) m/z (M+H)⁺=642.35 at retention time: 0.836 min, 100% ee) and Example 694: peak 2 ((SFC-MS) m/z (M+H)⁺=642.32 at retention time: 1.519 min, 100% ee).

Example 702 and 705\hat{ }: Example 703 was separated into its component isomers using SFC [Column: DAICEL CHIRALCEL OD 250 mm×30 mm×10 μm; Mobile phase: 50% CO₂/50% EtOH (0.1% NH₄OH); isocratic: 50% of Mobile Phase B; gradient time=30 min; flow rate: 80 mL/min]. The chiral purity was determined using SFC analytical method on IM-3 50 mm×4.6 mm×3 μm column; Mobile phase A: CO₂; Mobile phase B: EtOH with 0.2% of NH₃; isocratic: 50% of Mobile Phase B; flow rate: 3.5 mL/min; backpressure: 1500 psi; column temperature: 35 °C to provide Example 702: peak 1 ((SFC-MS) m/z (M+H)⁺=655.78 at retention time: 1.217 min, 100% ee) and Example 705: peak 2 ((SFC-MS) m/z (M+H)⁺=655.78 at retention time: 2.492 min, 100% ee).

Example 713 and 714\hat{ }: A mixture was separated into its component isomers using SFC [Column: Pirkle Covalent (R,R) Whelk-01 250 mm×21.1 mm×5 μm; Mobile phase: 40% MeOH (0.2% NH₄OH)/60% ACN; flow rate=75 mL/min, gradient time=70 min, backpressure=120 bar]. The chiral purity was determined using SFC analytical method on Regis (R,R) Whelk-01 50 mm×5 mm×3 μm column; Mobile phase A: MeOH (0.2% NH₄OH); Mobile phase B: ACN; isocratic gradient: 60% of Mobile Phase B; flow rate: 5.0 mL/min; backpressure: 120 bar, gradient time: 30 min to provide Example 714: peak 1 ((SFC-MS) m/z (M/2+H)⁺=308.2 at retention time: 16.09 min) and Example 713: peak 2 ((SFC-MS) m/z (M+H)⁺=615.4 at retention time: 16.43 min).

### Examples E1A-E426A

The compounds in Table 1A were prepared using similar chemistry as described above in the selected Examples 1-567 or as otherwise noted in the table and that are commercially available or that can be synthesized by literature methods to form the resultant compounds of the disclosure.

TABLE A

| | Method | | | |
|---|---|---|---|---|
| Example Number | of Synthesis | Structure/¹H NMR | Chem Draw Name | LC/MS m/z (M + H)⁺ |
| E1A | Example 567 | 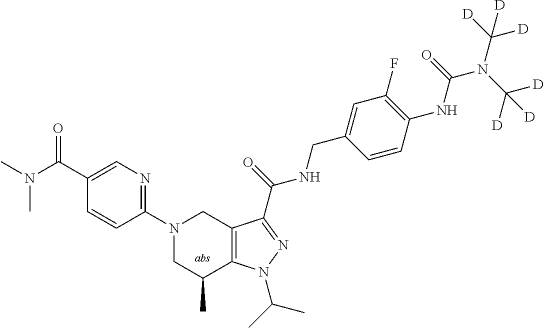 [SMILES: [2H]C([2H])([2H])N(C(=O)Nc1ccc(CNC(=O)c2nn(C(C)C)c3c2CN(c2ccc(C(=O)N(C)C)cn2)C[C@@H]3C)cc1F)C([2H])([2H])[2H]] | (S)-N-(4-(3,3- bis(methyl-d3)ureido)- 3-fluorobenzyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 571.4 |
| E2A | Example 567 | 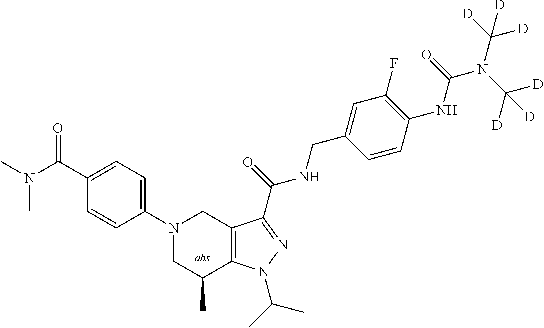 [SMILES: [2H]C([2H])([2H])N(C(=O)Nc1ccc(CNC(=O)c2nn(C(C)C)c3c2CN(c2ccc(C(=O)N(C)C)cc2)C[C@@H]3C)cc1F)C([2H])([2H])[2H]] | (S)-N-(4-(3,3- bis(methyl-d3)ureido)- 3-fluorobenzyl)-5-(4- (dimethylcarbamoyl) phenyl)-1-isopropyl-7- methyl-4,5,6,7- tetrahydro-1H- pyrazolo[4,3-c]pyridine- 3-carboxamide | 570.4 |
| E3A | Example 567 | 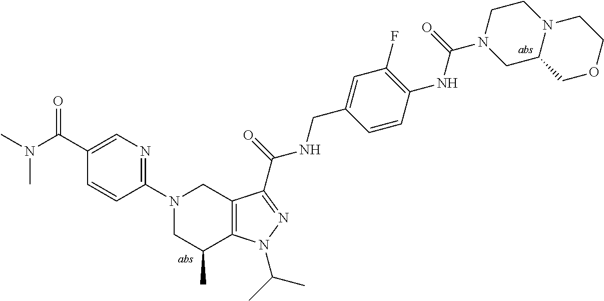 [SMILES: CC(C)n1nc(C(=O)NCc2ccc(NC(=O)N3CCN4CCOC[C@@H]4C3)c(F)c2)c2c1[C@@H](C)CN(c1ccc(C(=O)N(C)C)cn1)C2] | (S)-N-(4-(((S)-5-(5- (dimethylcarbamoyl) pyridin-2-yl)-1-isopropyl-7- methyl-4,5,6,7- tetrahydro-1H- pyrazolo[4,3-c]pyridine- 3-carboxamido)methyl)- 2- fluorophenyl)hexahydro- pyrazino[2,1- c][1,4]oxazine-8(1H)- carboxamide | 662.5 |
| E4A | Example 567 | 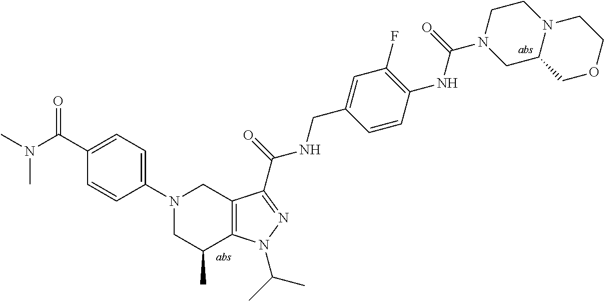 [SMILES: CC(C)n1nc(C(=O)NCc2ccc(NC(=O)N3CCN4CCOC[C@@H]4C3)c(F)c2)c2c1[C@@H](C)CN(c1ccc(C(=O)N(C)C)cc1)C2] | (S)-N-(4-(((S)-5-(4- (dimethylcarbamoyl) phenyl)-1-isopropyl)-7- methyl-4,5,6,7- tetrahydro-1H- pyrazolo[4,3-c]pyridine- 3-carboxamido)methyl)-2- fluorophenyl)hexahydro- pyrazino[2,1-c][1,4] oxazine-8(1H)- carboxamide | 661.5 |
| E5A | Example 567 | 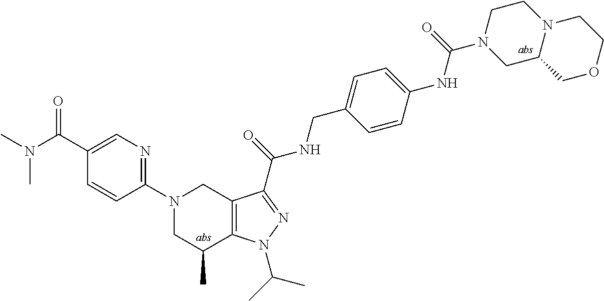 [SMILES: CC(C)n1nc(C(=O)NCc2ccc(NC(=O)N3CCN4CCOC[C@@H]4C3)cc2)c2c1[C@@H](C)CN(c1ccc(C(=O)N(C)C)cn1)C2] | (S)-N-(4-(((S)-5-(5- (dimethylcarbamoyl) pyridin-2-yl)-1-isopropyl-7- methyl-4,5,6,7- tetrahydro-1H- pyrazolo[4,3-c]pyridine- 3-carboxamido)methyl) phenyl)hexahydropyrazino [2,1-c][1,4]oxazine- 8(1H)-carboxamide | 644.5 |
| E6A | Example 567 | 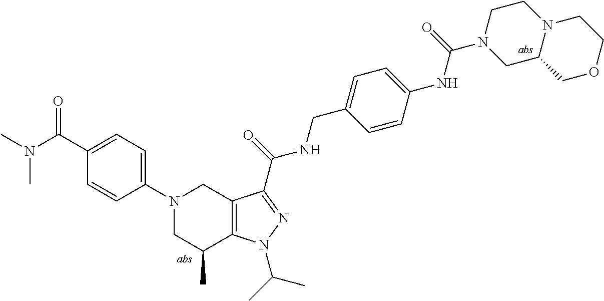 [SMILES: CC(C)n1nc(C(=O)NCc2ccc(NC(=O)N3CCN4CCOC[C@@H]4C3)cc2)c2c1[C@@H](C)CN(c1ccc(C(=O)N(C)C)cc1)C2] | (S)-N-(4-(((S)-5-(4- (dimethylcarbamoyl) phenyl)-1-isopropyl-7- methyl-4,5,6,7- tetrahydro-1H- pyrazolo[4,3-c]pyridine- 3-carboxamido)methyl) phenyl)hexahydropyrazino [2,1-c][1,4]oxazine- 8(1H)-carboxamide | 643.5 |
| E7A | Preparation 12/ Example 126^{b} | 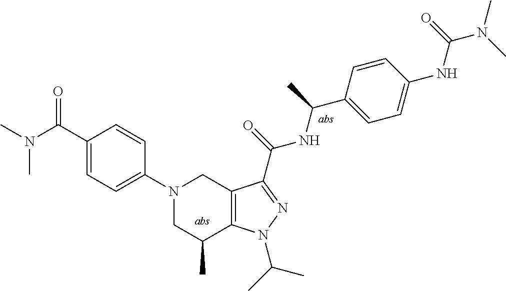 | (S)-5-(4- (dimethylcarbamoyl)phenyl)-N- ((S)-1-(4-(3,3- dimethylureido)phenyl)ethyl)- 1-isopropyl-7-methyl-4,5,6,7- tetrahydro-1H- pyrazolo[4,3-c]pyridine- 3-carboxamide | 560.5 |
| E8A | Preparation 12/ Example 126^{b} | 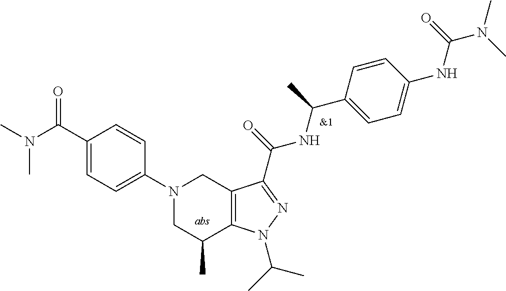 | (S)-5-(4- (dimethylcarbamoyl)phenyl)- N-((RS)-1-(4-(3,3- dimethylureido)phenyl) ethyl)-1-isopropyl-7- methyl-4,5,6,7- tetrahydro-1H- pyrazolo[4,3-c]pyridine- 3-carboxamide | 560.4 |
| E9A | Preparation 12/ Example 126^{b} | 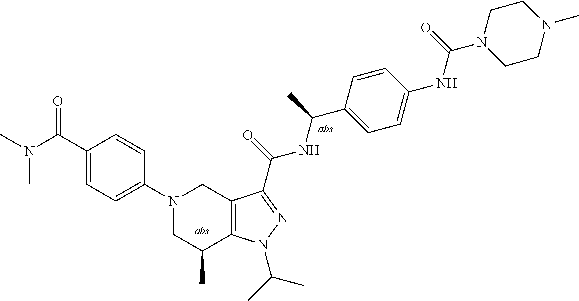 | (S)-5-(4- (dimethylcarbamoyl)phenyl)- 1-isopropyl-7- methyl-N-((S)-1-(4-(4- methylpiperazine-1- carboxamido)phenyl) ethyl)- 4,5,6,7-tetrahydro-1H- pyrazolo[4,3-c]pyridine- 3-carboxamide | 615.6 |
| E10A | Preparation 12/ Example 126^{b} | 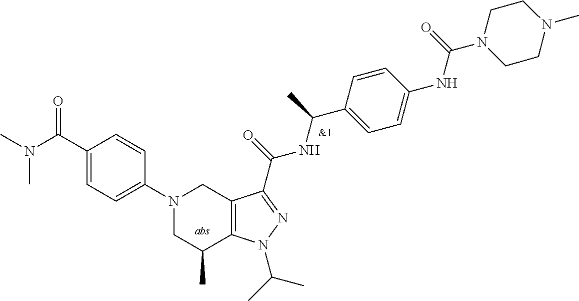 | (S)-5-(4- (dimethylcarbamoyl) phenyl)-1-isopropyl-7- methyl-N-((RS)-1-(4-(4- methylpiperazine-1- carboxamido)phenyl) ethyl)- 4,5,6,7-tetrahydro-1H- pyrazolo[4,3-c]pyridine- 3-carboxamide | 615.6 |
| E11A | Preparation 12/ Example 126 | 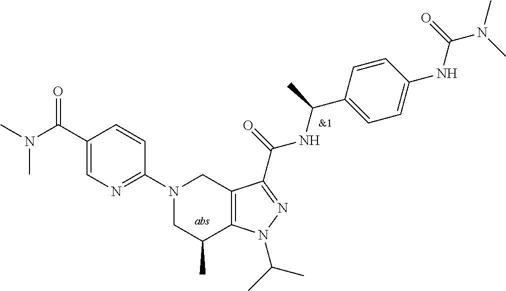 | (S)-5-(5- (dimethylcarbamoyl) pyridin-2-yl)-N-((RS)-1-(4- (3,3- dimethylureido)phenyl) ethyl)-1-isopropyl-7- methyl-4,5,6,7-tetrahydro-1H- pyrazolo[4,3-c]pyridine- 3-carboxamide | 561.5 |
| E12A | Preparation 12/ Example 126 | 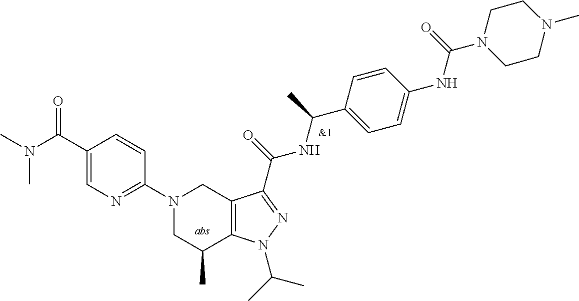 | (S)-5-(5- (dimethylcarbamoyl) pyridin-2-yl)-1-isopropyl-7- methyl-N-((RS)-1-(4-(4- methylpiperazine-1- carboxamido)phenyl) ethyl)-4,5,6,7-tetrahydro-1H- pyrazolo[4,3-c]pyridine- 3-carboxamide | 616.5 |
| E13A | Preparation 12/ Example 126^{b} | 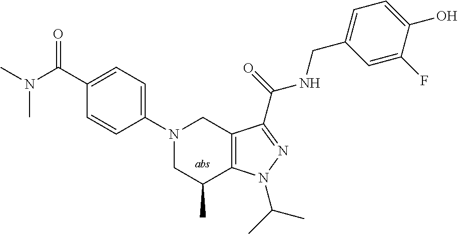 [SMILES: CC(C)n1nc(C(=O)NCc2ccc(O)c(F)c2)c2c1[C@@H](C)CN(c1ccc(C(=O)N(C)C)cc1)C2] | (S)-5-(4- (dimethylcarbamoyl) phenyl)-N-(3-fluoro-4- hydroxybenzyl)-1- isopropyl-7-methyl- 4,5,6,7-tetrahydro-1H- pyrazolo[4,3-c]pyridine- 3-carboxamide | 494.3 |
| E14A | Example 126 | 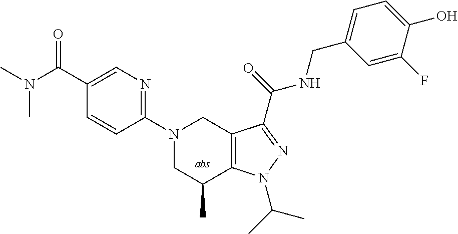 [SMILES: CC(C)n1nc(C(=O)NCc2ccc(O)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- hydroxybenzyl)-1- isopropyl-7-methyl- 4,5,6,7-tetrahydro-1H- pyrazolo[4,3-c]pyridine- 3-carboxamide | 494.2 |
| E15A | Example 126^{b} | 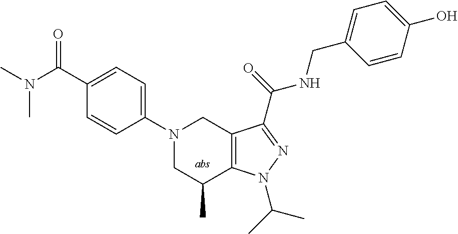 [SMILES: CC(C)n1nc(C(=O)NCc2ccc(O)cc2)c2c1[C@@H](C)CN(c1ccc(C(=O)N(C)C)cc1)C2] | (S)-5-(4- (dimethylcarbamoyl) phenyl)-N-(4- hydroxybenzyl)-1- isopropyl-7-methyl- 4,5,6,7-tetrahydro-1H- pyrazolo[4,3-c]pyridine- 3-carboxamide | 476.3 |
| E16A | Example 126 | 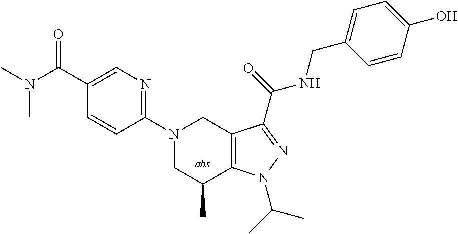 [SMILES: CC(C)n1nc(C(=O)NCc2ccc(O)cc2)c2c1[C@@H](C)CN(c1ccc(C(=O)N(C)C)cn1)C2] | (S)-5-(5- (dimethylcarbamoyl) pyridin-2-yl)-N-(4- hydroxybenzyl)-1- isopropyl-7-methyl- 4,5,6,7-tetrahydro-1H- pyrazolo[4,3-c]pyridine- 3-carboxamide | 477.3 |
| E17A | Example 193^{b} | 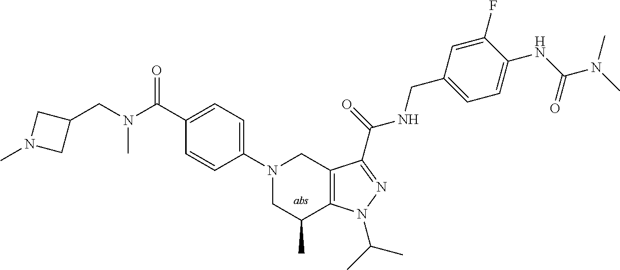 | (S)-N-(4-(3,3- dimethylureido)-3- fluorobenzyl)-1-isopropyl- 7-methyl-5-(4-(methyl((1- methylazetidin-3- yl)methyl)carbamoyl) phenyl)- 4,5,6,7-tetrahydro-1H- pyrazolo[4,3-c]pyridine- 3-carboxamide | 633.4 |
| E18A | Example 193^{b} | 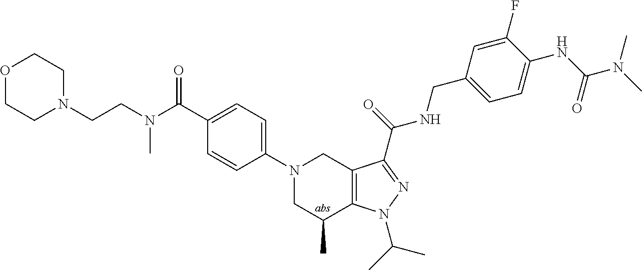 [SMILES: CC(C)n1nc(C(=O)NCc2ccc(NC(=O)N(C)C)c(F)c2)c2c1[C@@H](C)CN(c1ccc(C(=O)N(C)CCN3CCOCC3)cc1)C2] | (S)-N-(4-(3,3- dimethylureido)-3- fluorobenzyl)-1-isopropyl-7- methyl-5-(4-(methyl(2- morpholinoethyl)carbamoyl) phenyl)- 4,5,6,7-tetrahydro-1H- pyrazolo[4,3-c]pyridine- 3-carboxamide | 663.4 |
| E19A | Example 567 | 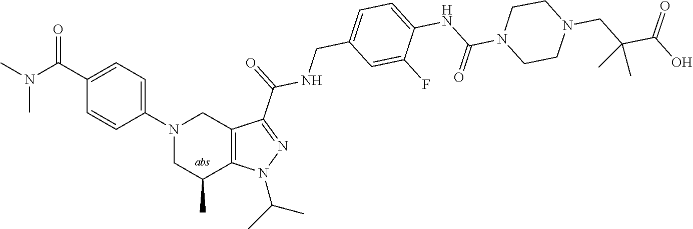 [SMILES: CC(C)n1nc(C(=O)NCc2ccc(NC(=O)N3CCN(CC(C)(C)C(=O)O)CC3)c(F)c2)c2c1[C@@H](C)CN(c1ccc(C(=O)N(C)C)cc1)C2] | (S)-3-(4-((4-((5-(4- (dimethylcarbamoyl) phenyl)-1-isopropyl-7- methyl-4,5,6,7-tetrahydro-1H- pyrazolo[4,3-c]pyridine- 3-carboxamido)methyl)- 2-fluorophenyl) carbamoyl)piperazin-1-yl)- 2,2-dimethylpropanoic acid | 705.5 |
| E20A | Example 126^{b} | 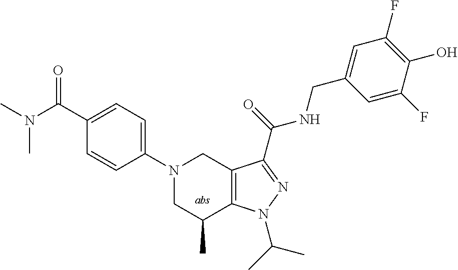 [SMILES: CC(C)n1nc(C(=O)NCc2cc(F)c(O)c(F)c2)c2c1[C@@H](C)CN(c1ccc(C(=O)N(C)C)cc1)C2] | (S)-N-(3,5-difluoro-4- hydroxybenzyl)-5-(4- (dimethylcarbamoyl) phenyl)-1-isopropyl-7- methyl-4,5,6,7-tetrahydro-1H- pyrazolo[4,3-c]pyridine- 3-carboxamide | 512.4 |
| E21A | Example 127 | 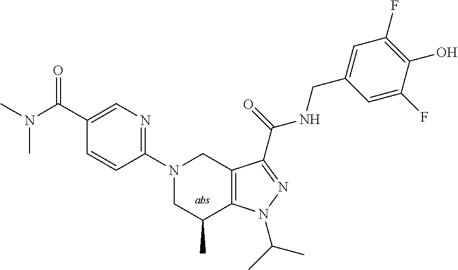 [SMILES: CC(C)n1nc(C(=O)NCc2cc(F)c(O)c(F)c2)c2c1[C@@H](C)CN(c1ccc(C(=O)N(C)C)cn1)C2] | (S)-N-(3,5-difluoro-4- hydroxybenzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 513.4 |
| E22A | Example 193^{b} | 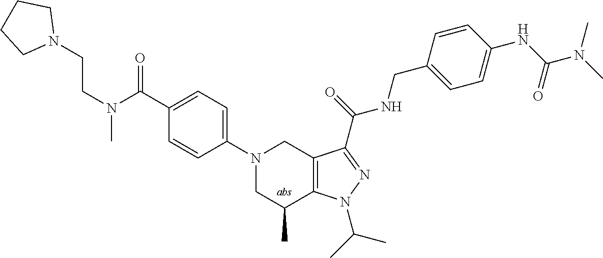 [SMILES: CC(C)n1nc(C(=O)NCc2ccc(NC(=O)N(C)C)cc2)c2c1[C@@H](C)CN(c1ccc(C(=O)N(C)CCN3CCCC3)cc1)C2] | (S)-N-(4-(3,3- dimethylureido)benzyl)- 1-isopropyl-7-methyl-5- (4-(methyl(2-(pyrrolidin- 1-yl)ethyl)carbamoyl) phenyl)-4,5,6,7-tetrahydro-1H- pyrazolo[4,3-c]pyridine- 3-carboxamide | 629.5 |
| E23A | Example 193^{b} | 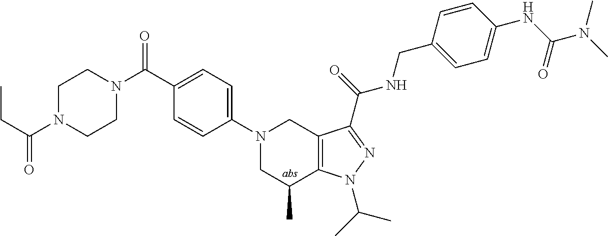 [SMILES: CCC(=O)N1CCN(C(=O)c2ccc(N3Cc4c(C(=O)NCc5ccc(NC(=O)N(C)C)cc5)nn(C(C)C)c4[C@@H](C)C3)cc2)CC1] | (S)-N-(4-(3,3- dimethylureido)benzyl)- 1-isopropyl-7-methyl-5- (4-(4- propionylpiperazine-1- carbonyl)phenyl)- 4,5,6,7-tetrahydro-1H- pyrazolo[4,3-c]pyridine- 3-carboxamide | 643.5 |
| E24A | Example 193^{b} | 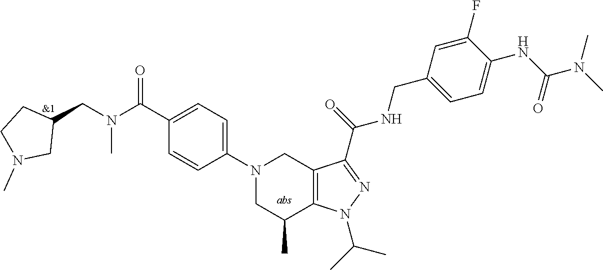 | (S)-N-(4-(3,3- dimethylureido)-3- fluorobenzyl)-1- isopropyl-7-methyl-5- (4-(methyl(((RS)-1- methylpyrrolidin-3- yl)methyl)carbamoyl) phenyl)-4,5,6,7-tetrahydro-1H- pyrazolo[4,3-c]pyridine- 3-carboxamide | 647.5 |
| E25A | Example 537 | 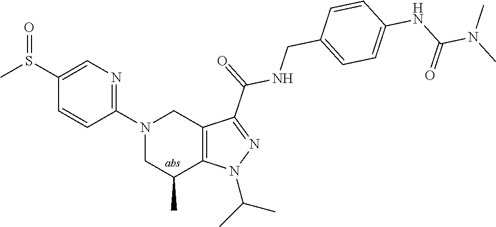 | (7S)-N-(4-(3,3- dimethylureido)benzyl)- 1-isopropyl-7-methyl-5- (5- (methylsulfinyl)pyridin- 2-yl)-4,5,6,7-tetrahydro-1H- pyrazolo[4,3-c]pyridine- 3-carboxamide | 538.3 |
| E26A | Example 236 | 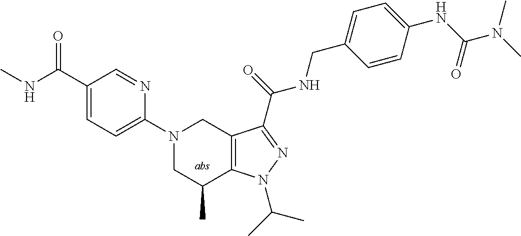 | (S)-N-(4-(3,3- dimethylureido)benzyl)- 1-isopropyl-7-methyl-5- (5- (methylcarbamoyl)pyridin- 2-yl)-4,5,6,7-tetrahydro-1H- pyrazolo[4,3-c]pyridine- 3-carboxamide | 533.3 |
| E27A | Example 567 | 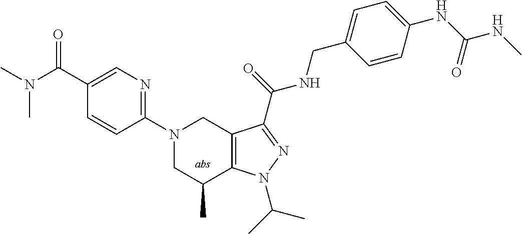 | (S)-1-isopropyl-7- methyl-5-(5- (methylcarbamoyl)pyridin- 2-yl)-N-(4-(3- methylureido)benzyl)- 4,5,6,7-tetrahydro-1H- pyrazolo[4,3-c]pyridine- 3-carboxamide | 519.2 |
| E28A | Example 126^{b} | 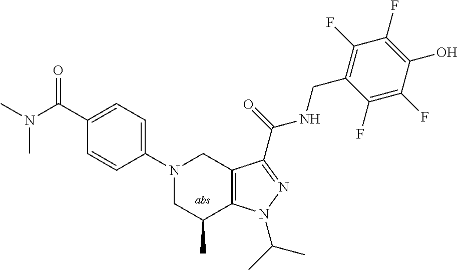 [SMILES: CC(C)n1nc(C(=O)NCc2c(F)c(F)c(O)c(F)c2F)c2c1[C@@H](C)CN(c1ccc(C(=O)N(C)C)cc1)C2] | (S)-5-(4- (dimethylcarbamoyl) phenyl)-1-isopropyl-7- methyl-N-(2,3,5,6- tetrafluoro-4- hydroxybenzyl)- 4,5,6,7-tetrahydro-1H- pyrazolo[4,3-c]pyridine- 3-carboxamide | 548.3 |
| E29A | Example 127 | 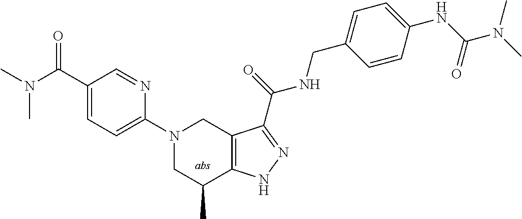 | (S)-5-(5- (dimethylcarbamoyl) pyridin-2-yl)-N-(4-(3,3- dimethylureido)benzyl)- 7-methyl-4,5,6,7-tetrahydro-1H- pyrazolo[4,3-c]pyridine- 3-carboxamide | 505.4 |
| | | ¹H NMR (400 MHz, (CD₃)₂SO) δ 13.09 | | |
| | | (s, 1H), 8.57-8.52 (m, 1H), 8.25-8.20 | | |
| | | (m, 2H), 7.63 (dd, 1H), 7.38 (d, 2H), | | |
| | | 7.16 (d, 2H), 6.87 (d, 1H), 4.98 (d, 1H), | | |
| | | 4.50-4.19 (m, 4H), 3.33-3.22 (m, | | |
| | | 1H), 3.09-3.01 (m, 1H), 2.96 (s, 6H), | | |
| | | 2.90 (s, 6H), 1.26 (d, 3H). | | |
| E30A | Example 127 | 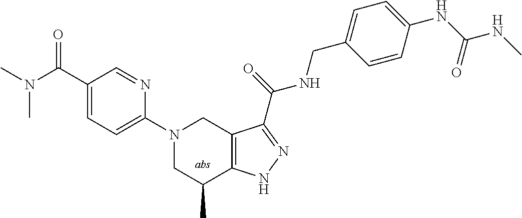 | (S)-5-(5- (dimethylcarbamoyl) pyridin-2-yl)-7-methyl-N- (4-(3- methylureido)benzyl)- 4,5,6,7-tetrahydro-1H- pyrazolo[4,3-c]pyridine- 3-carboxamide | 491.3 |
| E31A | Example 126 | 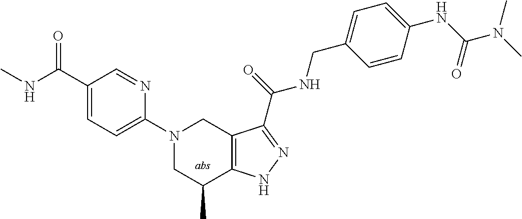 | (S)-N-(4-(3,3- dimethylureido)benzyl)- 7-methyl-5-(5- (methylcarbamoyl)pyridin- 2-yl)-4,5,6,7-tetrahydro-1H- pyrazolo[4,3-c]pyridine- 3-carboxamide | 491.3 |
| E32A | Example 567 | 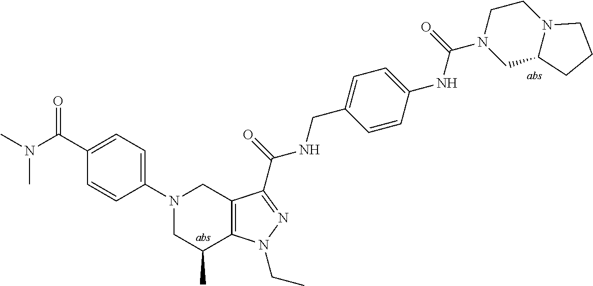 [SMILES: CCn1nc(C(=O)NCc2ccc(NC(=O)N3CCN4CCC[C@@H]4C3)cc2)c2c1[C@@H](C)CN(c1ccc(C(=O)N(C)C)cc1)C2] | (S)-5-(4- (dimethylcarbamoyl) phenyl)-1-ethyl-7-methyl- N-(4-((R)- octahydropyrrolo[1,2- a]pyrazine-2- carboxamido)benzyl)- 4,5,6,7-tetrahydro-1H- pyrazolo[4,3-c]pyridine- 3-carboxamide | 613.5 |
| E33A | Example 567 | 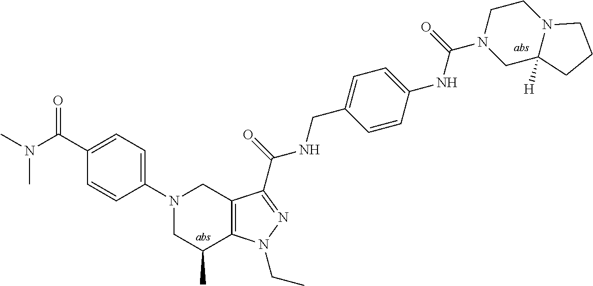 [SMILES: CCn1nc(C(=O)NCc2ccc(NC(=O)N3CCN4CCC[C@H]4C3)cc2)c2c1[C@@H](C)CN(c1ccc(C(=O)N(C)C)cc1)C2] | (S)-5-(4- (dimethylcarbamoyl) phenyl)-1-ethyl-7-methyl- N-(4-((S)- octahydropyrrolo[1,2-a] pyrazine-2- carboxamido)benzyl)- 4,5,6,7-tetrahydro-1H- pyrazolo[4,3-c]pyridine- 3-carboxamide | 613.5 |
| E34A | Example 126 | 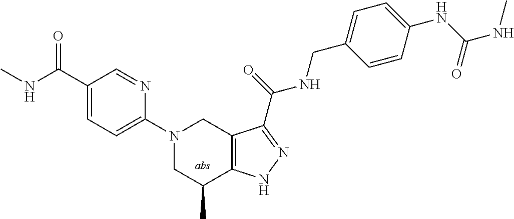 | (S)-7-methyl-5-(5- (methylcarbamoyl)pyridin- 2-yl)-N-(4-(3- methylureido)benzyl)- 4,5,6,7-tetrahydro-1H- pyrazolo[4,3-c]pyridine- 3-carboxamide | 477.4 |
| E35A | Example 485 | 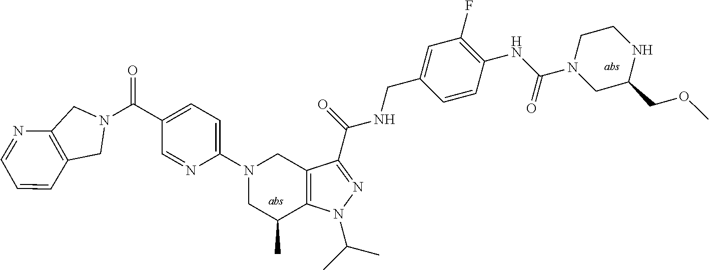 [SMILES: COC[C@H]1CN(C(=O)Nc2ccc(CNC(=O)c3nn(C(C)C)c4c3CN(c3ccc(C(=O)N5Cc6cccnc6C5)cn3)C[C@@H]4C)cc2F)CCN1] | (S)-5-(5-(6,7-dihydro- 5H-pyrrolo[3,4- b]pyridine-6- carbonyl)pyridin-2-yl)- N-(3-fluoro-4-((R)-3- (methoxymethyl) piperazine-1- carboxamido)benzyl)-1- isopropyl-7-methyl- 4,5,6,7-tetrahydro-1H- pyrazolo[4,3-c]pyridine- 3-carboxamide | 725.5 |
| E36A | Example 485 | 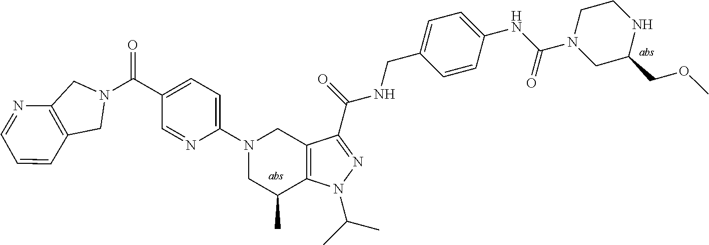 [SMILES: COC[C@H]1CN(C(=O)Nc2ccc(CNC(=O)c3nn(C(C)C)c4c3CN(c3ccc(C(=O)N5Cc6cccnc6C5)cn3)C[C@@H]4C)cc2)CCN1] | (S)-5-(5-(6,7-dihydro- 5H-pyrrolo[3,4-b] pyridine-6- carbonyl)pyridin-2-yl)- 1-isopropyl-N-(4-((R)-3- (methoxymethyl) piperazine-1- carboxamido)benzyl)-7- methyl-4,5,6,7-tetrahydro-1H- pyrazolo[4,3-c]pyridine- 3-carboxamide | 707.4 |
| E37A | Example 396 | 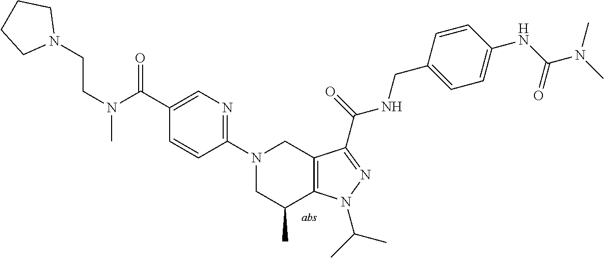 [SMILES: CC(C)n1nc(C(=O)NCc2ccc(NC(=O)N(C)C)cc2)c2c1[C@@H](C)CN(c1ccc(C(=O)N(C)CCN3CCCC3)cn1)C2] | (S)-N-(4-(3,3- dimethylureido)benzyl)- 1-isopropyl-7-methyl-5- (5-(methyl(2-(pyrrolidin- 1-yl)ethyl)carbamoyl) pyridin-2-yl)- 4,5,6,7-tetrahydro-1H- pyrazolo[4,3-c]pyridine- 3-carboxamide | 630.4 |
| E38A | Example 395 | 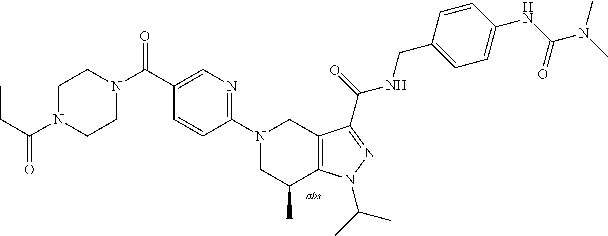 [SMILES: CCC(=O)N1CCN(C(=O)c2ccc(N3Cc4c(C(=O)NCc5ccc(NC(=O)N(C)C)cc5)nn(C(C)C)c4[C@@H](C)C3)nc2)CC1] | (S)-N-(4-(3,3- dimethylureido)benzyl)- 1-isopropyl-7-methyl-5- (5-(4- propionylpiperazine-1- carbonyl)pyridin-2-yl)- 4,5,6,7-tetrahydro-1H- pyrazolo[4,3-c]pyridine- 3-carboxamide | 644.4 |
| E39A | Example 485 | 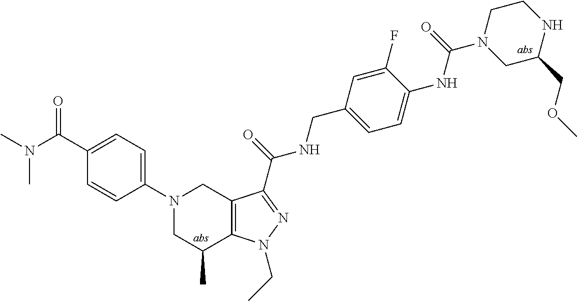 | (S)-5-(4- (dimethylcarbamoyl) phenyl)-1-ethyl-N-(3- fluoro-4-((R)-3- (methoxymethyl) piperazine-1- carboxamido)benzyl)-7- methyl-4,5,6,7-tetrahydro-1H- pyrazolo[4,3-c]pyridine- 3-carboxamide | 635.5 |
| E40A | Example 485 | 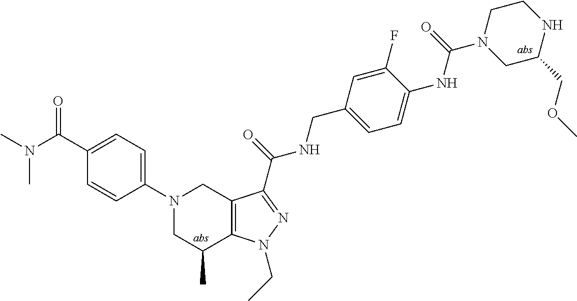 | (S)-5-(4- (dimethylcarbamoyl) phenyl)-1-ethyl-N-(3- fluoro-4-((S)-3- (methoxymethyl)piperazine-1- carboxamido)benzyl)-7- methyl-4,5,6,7-tetrahydro-1H- pyrazolo[4,3-c]pyridine- 3-carboxamide | 635.5 |
| E41A | Example 126^{b} | 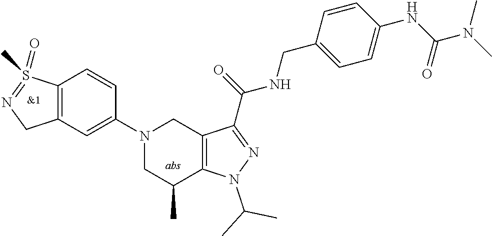 | (S)-N-(4-(3,3- dimethylureido)benzyl)- 1-isopropyl-7-methyl-5- ((R)-1-methyl-1-oxido- 3H-1l4- benzo[d]isothiazol-5- yl)-4,5,6,7-tetrahydro-1H- pyrazolo[4,3-c]pyridine- 3-carboxamide | 564.3 |
| E42A | Example 126 | 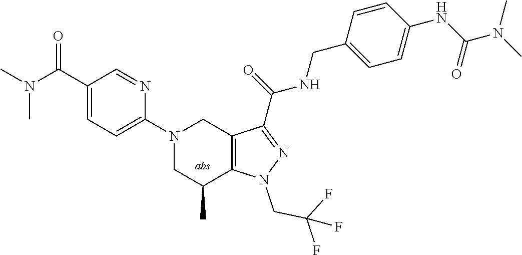 | rac-(R)-5-(5- (dimethylcarbamoyl) pyridin-2-yl)-N-(4-(3,3- dimethylureido)benzyl)- 7-methyl-1-(2,2,2- trifluoroethyl)- 4,5,6,7-tetrahydro-1H- pyrazolo[4,3-c]pyridine- 3-carboxamide | 587.5 |
| E43A | Example 193^{b} | 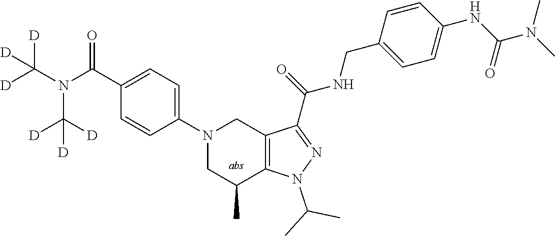 [SMILES: [2H]C([2H])([2H])N(C(=O)c1ccc(N2Cc3c(C(=O)NCc4ccc(NC(=O)N(C)C)cc4)nn(C(C)C)c3[C@@H](C)C2)cc1)C([2H])([2H])[2H]] | (S)-5-(4-(bis(methyl- d3)carbamoyl)phenyl)- N-(4-(3,3- dimethylureido)benzyl)- 1-isopropyl-7-methyl- 4,5,6,7-tetrahydro-1H- pyrazolo[4,3-c]pyridine- 3-carboxamide | 552.4 |
| E44A | Example 193^{b} | 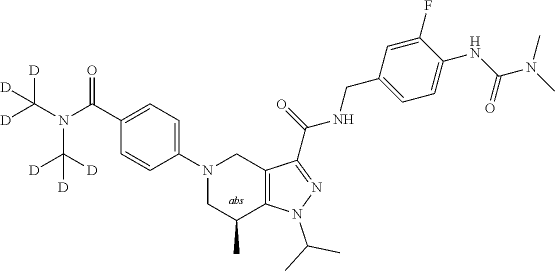 [SMILES: [2H]C([2H])([2H])N(C(=O)c1ccc(N2Cc3c(C(=O)NCc4ccc(NC(=O)N(C)C)c(F)c4)nn(C(C)C)c3[C@@H](C)C2)cc1)C([2H])([2H])[2H]] | (S)-5-(4-(bis(methyl- d3)carbamoyl)phenyl)- N-(4-(3,3- dimethylureido)-3- fluorobenzyl)-1- isopropyl-7-methyl- 4,5,6,7-tetrahydro-1H- pyrazolo[4,3-c]pyridine- 3-carboxamide | 570.4 |
| E45A | Example 127 | 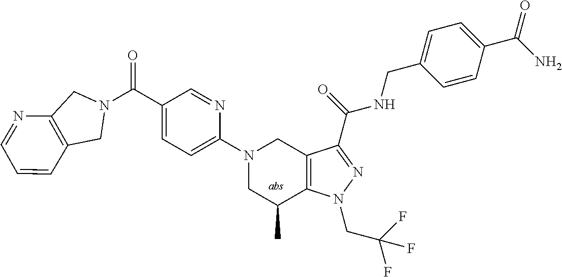 [SMILES: C[C@H]1CN(c2ccc(C(=O)N3Cc4cccnc4C3)cn2)Cc2c(C(=O)NCc3ccc(C(N)=O)cc3)nn(CC(F)(F)F)c21] | (S)-N-(4- carbamoylbenzyl)-5-(5- (6,7-dihydro-5H- pyrrolo[3,4-b]pyridine- 6-carbonyl)pyridin-2- yl)-7-methyl-1-(2,2,2- trifluoroethyl)- 4,5,6,7-tetrahydro-1H- pyrazolo[4,3-c]pyridine- 3-carboxamide | 619.3 |
| E46A | Preparation 12/ Example 127 | 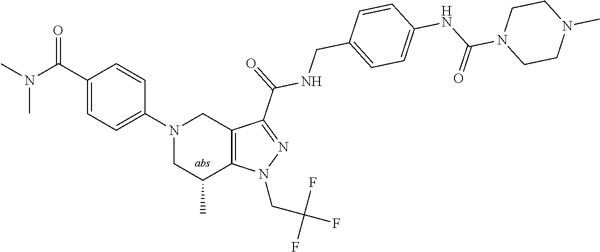 | (R)-5-(4- (dimethylcarbamoyl) phenyl)-7-methyl-N-(4-(4- methylpiperazine-1- carboxamido)benzyl)-1- (2,2,2-trifluoroethyl)- 4,5,6,7-tetrahydro-1H- pyrazolo[4,3-c]pyridine- 3-carboxamide | 641.4 |
| E47A | Example 127 | 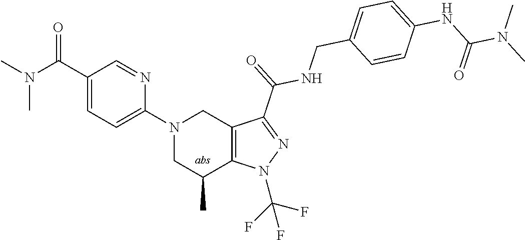 | (S)-5-(5- (dimethylcarbamoyl) pyridin-2-yl)-N-(4-(3,3- dimethylureido)benzyl)- 7-methyl-1-(trifluoromethyl)- 4,5,6,7-tetrahydro-1H- pyrazolo[4,3-c]pyridine- 3-carboxamide | 573.3 |
| E48A | Preparation 12/ Example 126^{b} | 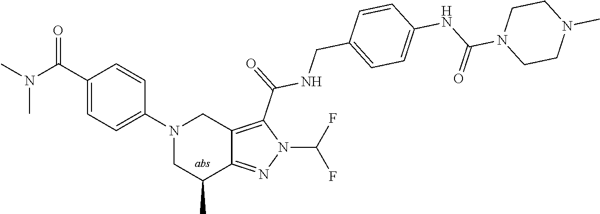 | (S)-2-(difluoromethyl)-5-(4- (dimethylcarbamoyl) phenyl)-7-methyl-N-(4-(4- methylpiperazine-1- carboxamido)benzyl)- 4,5,6,7-tetrahydro-1H- pyrazolo[4,3-c]pyridine- 3-carboxamide | 609.5 |
| E49A | Preparation 12/ Example 126^{b} | 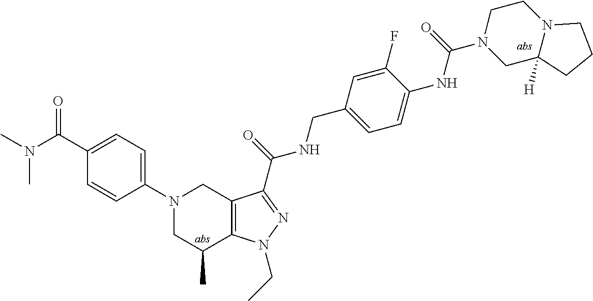 [SMILES: CCn1nc(C(=O)NCc2ccc(NC(=O)N3CCN4CCC[C@H]4C3)c(F)c2)c2c1[C@@H](C)CN(c1ccc(C(=O)N(C)C)cc1)C2] | (S)-5-(4- (dimethylcarbamoyl) phenyl)-1-ethyl-N-(3- fluoro-4-((S)- octahydropyrrolo[1,2- a]pyrazine-2- carboxamido)benzyl)-7- methyl-4,5,6,7-tetrahydro-1H- pyrazolo[4,3-c]pyridine- 3-carboxamide | 631.4 |
| E50A | Example 127 | 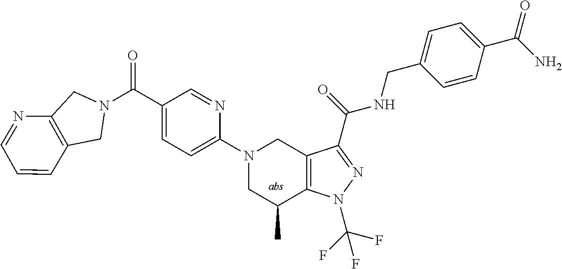 [SMILES: C[C@H]1CN(c2ccc(C(=O)N3Cc4cccnc4C3)cn2)Cc2c(C(=O)NCc3ccc(C(N)=O)cc3)nn(C(F)(F)F)c21] | (S)-N-(4- carbamoylbenzyl)-5-(5- (6,7-dihydro-5H- pyrrolo[3,4-b]pyridine- 6-carbonyl)pyridin-2- yl)-7-methyl-1- (trifluoromethyl)- 4,5,6,7-tetrahydro-1H- pyrazolo[4,3-c]pyridine- 3-carboxamide | 631.4 |
| E51A | Example 127 | 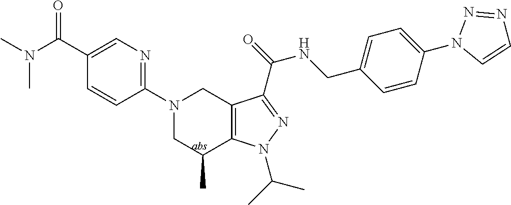 [SMILES: CC(C)n1nc(C(=O)NCc2ccc(-n3ccnn3)cc2)c2c1[C@@H](C)CN(c1ccc(C(=O)N(C)C)cn1)C2] | (S)-N-(4-(1H-1,2,3- triazol-1-yl)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 528.4 |
| E52A | Example 567 | 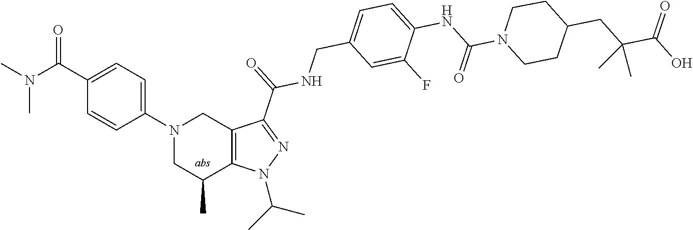 [SMILES: CC(C)n1nc(C(=O)NCc2ccc(NC(=O)N3CCC(CC(C)(C)C(=O)O)CC3)c(F)c2)c2c1[C@@H](C)CN(c1ccc(C(=O)N(C)C)cc1)C2] | (S)-3-(1-((4-((5-(4- (dimethylcarbamoyl)phenyl)- 1-isopropyl-7-methyl- 4,5,6,7-tetrahydro-1H- pyrazolo[4,3-c]pyridine- 3-carboxamido)methyl)-2- fluorophenyl)carbamoyl) piperidin-4-yl)-2,2- dimethylpropanoic acid | 704.5 |
| E53A | Example 193^{ab} | 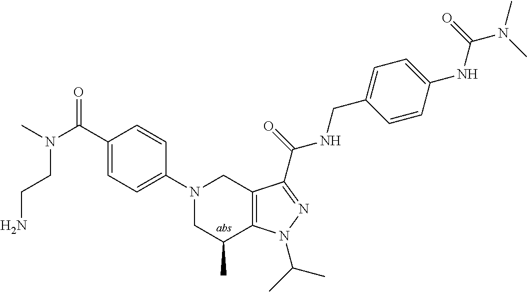 | (S)-5-(4-((2- aminoethyl)(methyl)carbamoyl) phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 575.5 |
| E54A | Example 193^{ab} | 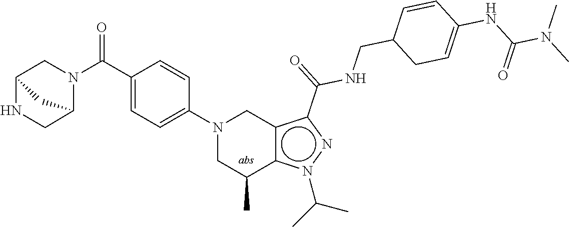 | (7S)-5-(4-((1R,4R)-2,5- diazabicyclo[2.2.1]heptane- 2-carbonyl)phenyl)-N-((4- (3,3-dimethylureido) cyclohexa-2,4-dien-1- yl)methyl)-1-isopropyl- 7-methyl-4,5,6,7-tetrahydro- 1H-pyrazolo[4,3-c]pyridine- 3-carboxamide | 599.5 |
| E55A | Example 193^{ab} | 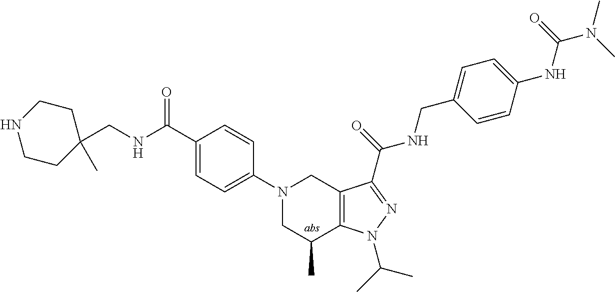 [SMILES: CC(C)n1nc(C(=O)NCc2ccc(NC(=O)N(C)C)cc2)c2c1[C@@H](C)CN(c1ccc(C(=O)NCC3(C)CCNCC3)cc1)C2] | (7S)-5-(4-((1R,4R)-2,5- diazabicyclo[2.2.1]heptane-2- carbonyl)phenyl)-N-((4-(3,3- dimethylureido)cyclohexa-2,4- dien-1-yl)methyl)-1- isopropyl-7-methyl- 4,5,6,7-tetrahydro-1H- pyrazolo[4,3-c]pyridine- 3-carboxamide | 629.5 |
| E56A | Example 193^{ab} | 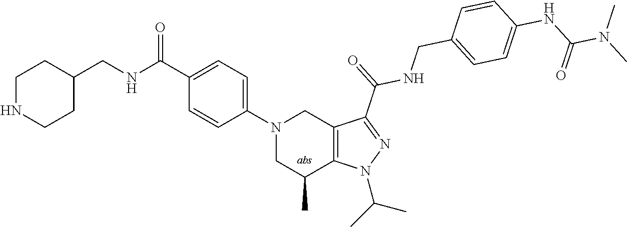 [SMILES: CC(C)n1nc(C(=O)NCc2ccc(NC(=O)N(C)C)cc2)c2c1[C@@H](C)CN(c1ccc(C(=O)NCC3CCNCC3)cc1)C2] | (S)-N-(4-(3,3- dimethylureido)benzyl)- 1-isopropyl-7-methyl-5- (4-((piperidin-4- ylmethyl)carbamoyl) phenyl)-4,5,6,7-tetrahydro-1H- pyrazolo[4,3-c]pyridine- 3-carboxamide | 615.5 |
| E57A | Example 193^{ab} | 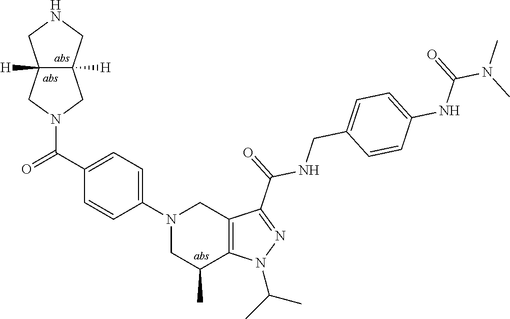 [SMILES: CC(C)n1nc(C(=O)NCc2ccc(NC(=O)N(C)C)cc2)c2c1[C@@H](C)CN(c1ccc(C(=O)N3C[C@@H]4CNC[C@H]4C3)cc1)C2] | (S)-N-(4-(3,3- dimethylureido)benzyl)- 1-isopropyl-7-methyl-5- (4-((3aS,6aS)- octahydropyrrolo[3,4- c]pyrrole-2- carbonyl)phenyl)- 4,5,6,7-tetrahydro-1H- pyrazolo[4,3-c]pyridine- 3-carboxamide | 613.5 |
| E58A | Example 193^{ab} | 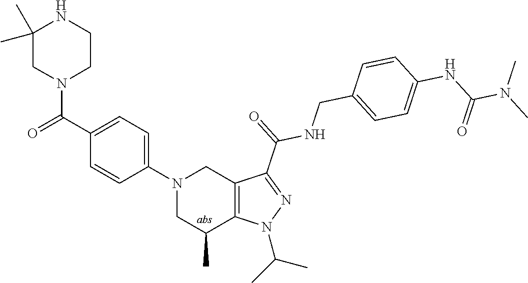 | (S)-5-(4-(3,3- dimethylpiperazine-1- carbonyl)phenyl)-N-(4- (3,3-dimethylureido)benzyl)- 1-isopropyl-7-methyl- 4,5,6,7-tetrahydro-1H- pyrazolo[4,3-c]pyridine- 3-carboxamide | 615.6 |
| E59A | Example 193^{ab} | 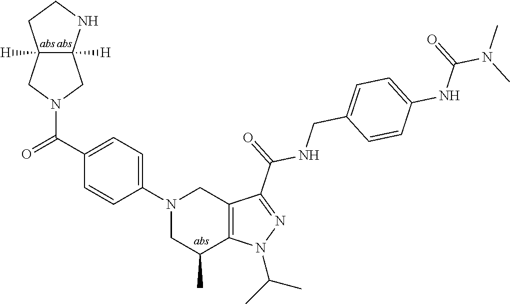 [SMILES: CC(C)n1nc(C(=O)NCc2ccc(NC(=O)N(C)C)cc2)c2c1[C@@H](C)CN(c1ccc(C(=O)N3C[C@H]4CCN[C@H]4C3)cc1)C2] | (S)-N-(4-(3,3- dimethylureido)benzyl)- 1-isopropyl-7-methyl-5- (4-((3aR,6aR)- octahydropyrrolo[3,4- b]pyrrole-5- carbonyl)phenyl)- 4,5,6,7-tetrahydro-1H- pyrazolo[4,3-c]pyridine- 3-carboxamide | 613.5 |
| E60A | Example 193^{ab} | 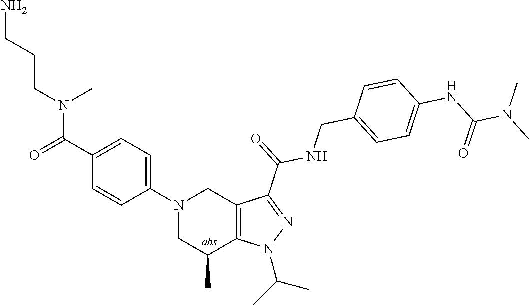 [SMILES: CC(C)n1nc(C(=O)NCc2ccc(NC(=O)N(C)C)cc2)c2c1[C@@H](C)CN(c1ccc(C(=O)N(C)CCCN)cc1)C2] | (S)-5-(4-((3- aminopropyl)(methyl) carbamoyl)phenyl)-N-(4- (3,3-dimethylureido)benzyl)- 1-isopropyl-7-methyl- 4,5,6,7-tetrahydro-1H- pyrazolo[4,3-c]pyridine- 3-carboxamide | 589.5 |
| E61A | Example 193^{ab} | 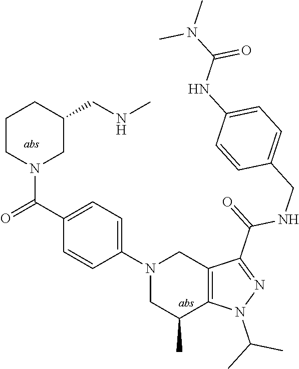 [SMILES: CNC[C@H]1CCCN(C(=O)c2ccc(N3Cc4c(C(=O)NCc5ccc(NC(=O)N(C)C)cc5)nn(C(C)C)c4[C@@H](C)C3)cc2)C1] | (S)-N-(4-(3,3- dimethylureido)benzyl)- 1-isopropyl-7-methyl-5- (4-((R)-3- ((methylamino)methyl) piperidine-1- carbonyl)phenyl)- 4,5,6,7-tetrahydro-1H- pyrazolo[4,3-c]pyridine- 3-carboxamide | 629.5 |
| E62A | Example 193^{ab} | 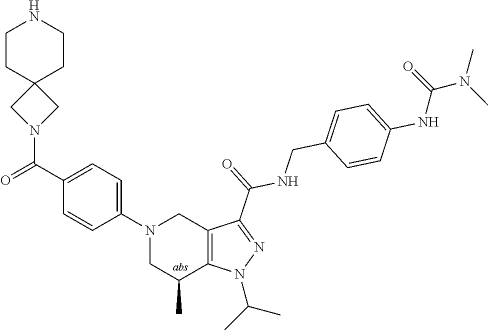 [SMILES: CC(C)n1nc(C(=O)NCc2ccc(NC(=O)N(C)C)cc2)c2c1[C@@H](C)CN(c1ccc(C(=O)N3CC4(CCNCC4)C3)cc1)C2] | (S)-5-(4-(2,7- diazaspiro[3.5]nonane- 2-carbonyl)phenyl)-N- (4-(3,3- dimethylureido)benzyl)- 1-isopropyl-7-methyl- 4,5,6,7-tetrahydro-1H- pyrazolo[4,3-c]pyridine- 3-carboxamide | 627.6 |
| E63A | Example 193^{ab} | 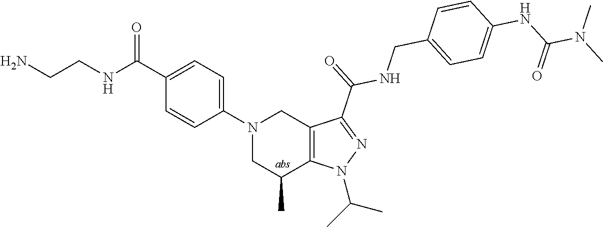 [SMILES: CC(C)n1nc(C(=O)NCc2ccc(NC(=O)N(C)C)cc2)c2c1[C@@H](C)CN(c1ccc(C(=O)NCCN)cc1)C2] | (S)-5-(4-((2- aminoethyl)carbamoyl) phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 561.5 |
| E64A | Example 193^{ab} | 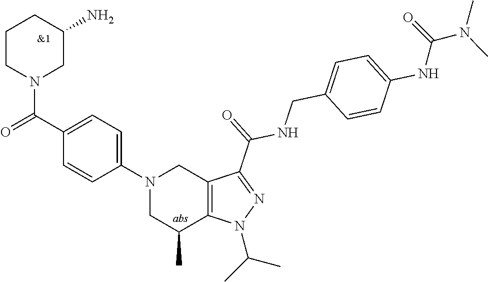 [SMILES: CC(C)n1nc(C(=O)NCc2ccc(NC(=O)N(C)C)cc2)c2c1[C@@H](C)CN(c1ccc(C(=O)N3CCC[C@H](N)C3)cc1)C2] | (S)-5-(4-((RS)-3- aminopiperidine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 601.5 |
| E65A | Example 193^{ab} | 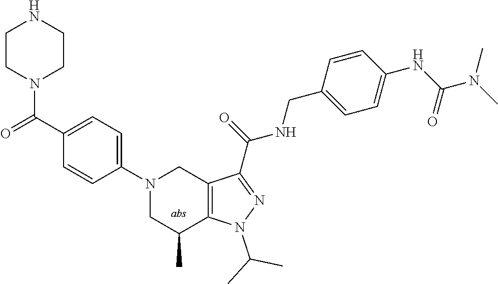 [SMILES: CC(C)n1nc(C(=O)NCc2ccc(NC(=O)N(C)C)cc2)c2c1[C@@H](C)CN(c1ccc(C(=O)N3CCNCC3)cc1)C2] | (S)-N-(4-(3,3- dimethylureido)benzyl)- 1-isopropyl-7-methyl-5- (4-(piperazine-1- carbonyl)phenyl)- 4,5,6,7-tetrahydro-1H- pyrazolo[4,3-c]pyridine- 3-carboxamide | 587.5 |
| E66A | Example 193^{ab} | 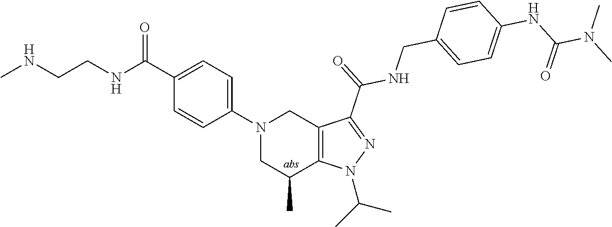 | (S)-N-(4-(3,3- dimethylureido)benzyl)- 1-isopropyl-7-methyl-5- (4-((2- (methylamino)ethyl) carbamoyl)phenyl)- 4,5,6,7-tetrahydro-1H- pyrazolo[4,3-c]pyridine- 3-carboxamide | 575.4 |
| E67A | Example 193^{ab} | 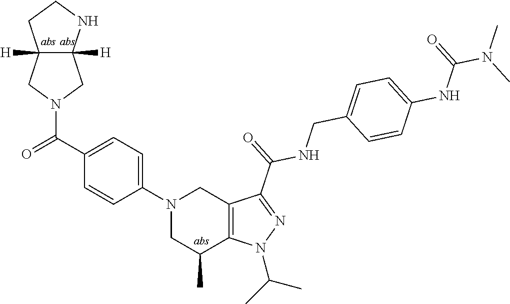 [SMILES: CC(C)n1nc(C(=O)NCc2ccc(NC(=O)N(C)C)cc2)c2c1[C@@H](C)CN(c1ccc(C(=O)N3C[C@@H]4CCN[C@@H]4C3)cc1)C2] | (S)-N-(4-(3,3- dimethylureido)benzyl)- 1-isopropyl-7-methyl-5- (4-((3aS,6aS)- octahydropyrrolo[3,4- b]pyrrole-5- carbonyl)phenyl)- 4,5,6,7-tetrahydro-1H- pyrazolo[4,3-c]pyridine- 3-carboxamide | 613.6 |
| E68A | Example 193^{ab} | 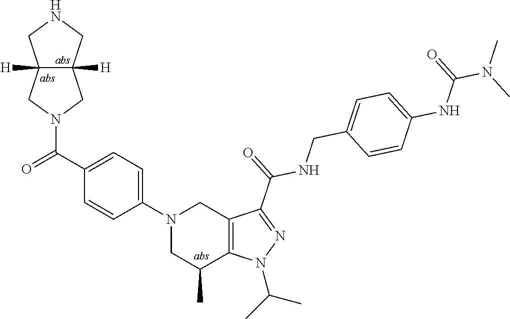 [SMILES: CC(C)n1nc(C(=O)NCc2ccc(NC(=O)N(C)C)cc2)c2c1[C@@H](C)CN(c1ccc(C(=O)N3C[C@H]4CNC[C@H]4C3)cc1)C2] | (S)-N-(4-(3,3- dimethylureido)benzyl)- 1-isopropyl-7-methyl-5- (4-((3aR,6aS)- octahydropyrrolo[3,4- c]pyrrole-2- carbonyl)phenyl)- 4,5,6,7-tetrahydro-1H- pyrazolo[4,3-c]pyridine- 3-carboxamide | 613.5 |
| E69A | Example 193^{ab} | 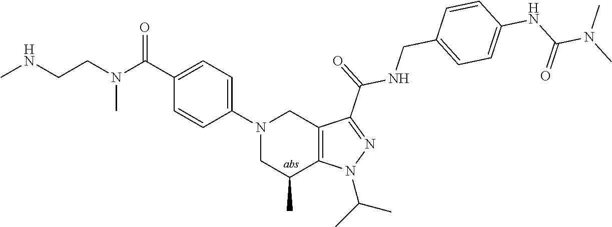 | (S)-N-(4-(3,3- dimethylureido)benzyl)- 1-isopropyl-7-methyl-5- (4-(methyl(2- (methylamino)ethyl) carbamoyl)phenyl)- 4,5,6,7-tetrahydro-1H- pyrazolo[4,3-c]pyridine- 3-carboxamide | 589.5 |
| E70A | Example 193^{ab} | 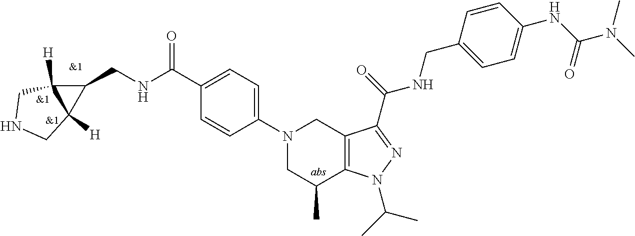 [SMILES: CC(C)n1nc(C(=O)NCc2ccc(NC(=O)N(C)C)cc2)c2c1[C@@H](C)CN(c1ccc(C(=O)NC[C@H]3[C@@H]4CNC[C@@H]43)cc1)C2] | (S)-5-(4- ((((1RS,5SR,6SR)-3- azabicyclo[3.1.0]hexan-6- yl)methyl)carbamoyl) phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 613.5 |
| E71A | Example 193^{ab} | 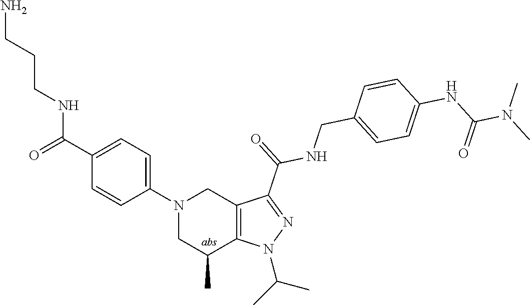 [SMILES: CC(C)n1nc(C(=O)NCc2ccc(NC(=O)N(C)C)cc2)c2c1[C@@H](C)CN(c1ccc(C(=O)NCCCN)cc1)C2] | (S)-5-(4-((3- aminopropyl)carbamoyl) phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 575.5 |
| E72A | Example 193^{b} | 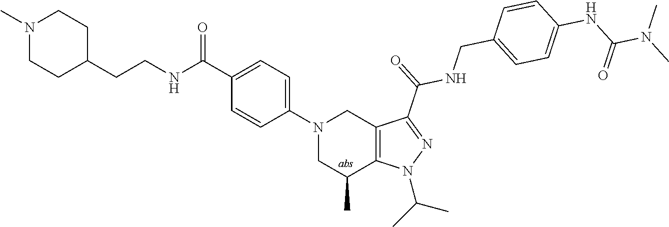 | (S)-N-(4-(3,3- dimethylureido)benzyl)- 1-isopropyl-7-methyl-5- (4-((2-(1- methylpiperidin-4- yl)ethyl)carbamoyl) phenyl)-4,5,6,7-tetrahydro-1H- pyrazolo[4,3-c]pyridine- 3-carboxamide | 643.6 |
| E73A | Example 193^{b} | 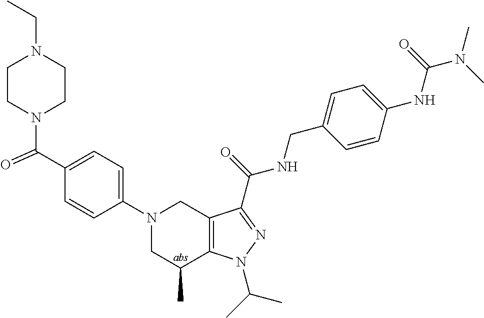 [SMILES: CCN1CCN(C(=O)c2ccc(N3Cc4c(C(=O)NCc5ccc(NC(=O)N(C)C)cc5)nn(C(C)C)c4[C@@H](C)C3)cc2)CC1] | (S)-N-(4-(3,3- dimethylureido)benzyl)- 5-(4-(4-ethylpiperazine- 1-carbonyl)phenyl)-1- isopropyl-7-methyl- 4,5,6,7-tetrahydro-1H- pyrazolo[4,3-c]pyridine- 3-carboxamide | 615.5 |
| E74A | Example 193^{b} | 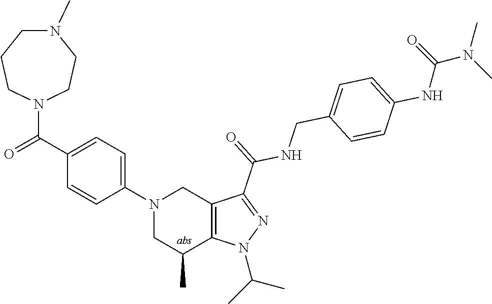 [SMILES: CC(C)n1nc(C(=O)NCc2ccc(NC(=O)N(C)C)cc2)c2c1[C@@H](C)CN(c1ccc(C(=O)N3CCCN(C)CC3)cc1)C2] | (S)-N-(4-(3,3- dimethylureido)benzyl)- 1-isopropyl-7-methyl-5- (4-(4-methyl-1,4- diazepane-1- carbonyl)phenyl)- 4,5,6,7-tetrahydro-1H- pyrazolo[4,3-c]pyridine- 3-carboxamide | 615.5 |
| E75A | Example 193^{ab} | 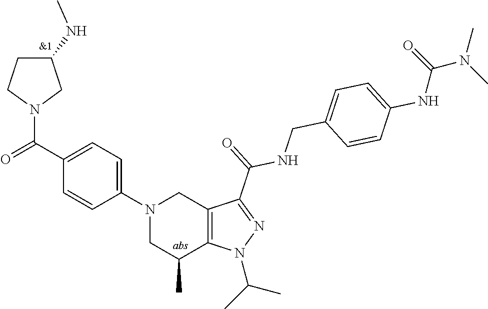 [SMILES: CN[C@H]1CCN(C(=O)c2ccc(N3Cc4c(C(=O)NCc5ccc(NC(=O)N(C)C)cc5)nn(C(C)C)c4[C@@H](C)C3)cc2)C1] | (S)-N-(4-(3,3- dimethylureido)benzyl)- 1-isopropyl-7-methyl-5- (4-((RS)-3- (methylamino)pyrrolidine-1- carbonyl)phenyl)- 4,5,6,7-tetrahydro-1H- pyrazolo[4,3-c]pyridine- 3-carboxamide | 601.5 |
| E76A | Example 193^{b} | 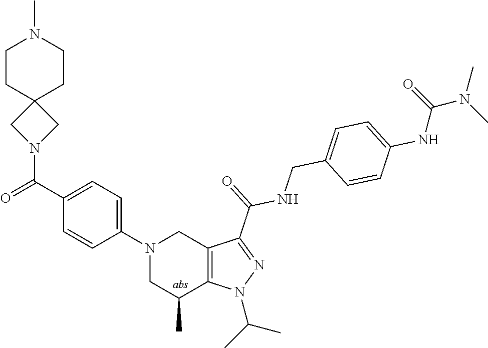 [SMILES: CC(C)n1nc(C(=O)NCc2ccc(NC(=O)N(C)C)cc2)c2c1[C@@H](C)CN(c1ccc(C(=O)N3CC4(CCN(C)CC4)C3)cc1)C2] | (S)-N-(4-(3,3- dimethylureido)benzyl)- 1-isopropyl-7-methyl-5- (4-(7-methyl-2,7- diazaspiro[3.5]nonane- 2-carbonyl)phenyl)- 4,5,6,7-tetrahydro-1H- pyrazolo[4,3-c]pyridine- 3-carboxamide | 641.5 |
| E77A | Example 193^{ab} | 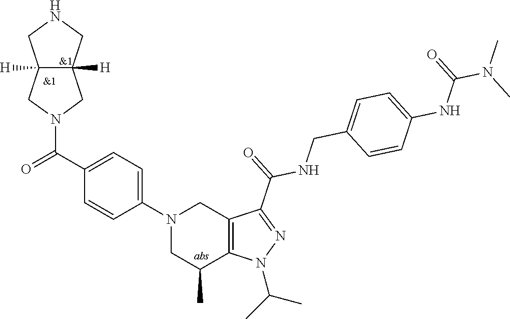 [SMILES: CC(C)n1nc(C(=O)NCc2ccc(NC(=O)N(C)C)cc2)c2c1[C@@H](C)CN(c1ccc(C(=O)N3C[C@H]4CNC[C@@H]4C3)cc1)C2] | (S)-N-(4-(3,3- dimethylureido)benzyl)- 1-isopropyl-7-methyl-5- (4-((3aRS,6aRS)- octahydropyrrolo[3,4- c]pyrrole-2- carbonyl)phenyl)- 4,5,6,7-tetrahydro-1H- pyrazolo[4,3-c]pyridine- 3-carboxamide | 613.5 |
| E78A | Example 193^{ab} | 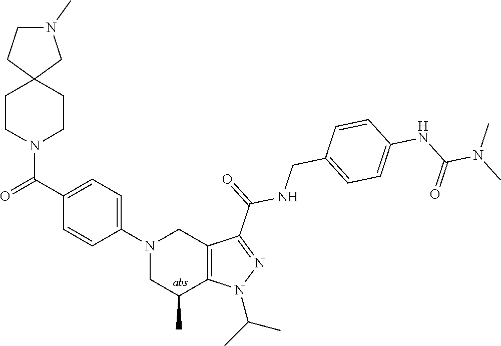 [SMILES: CC(C)n1nc(C(=O)NCc2ccc(NC(=O)N(C)C)cc2)c2c1[C@@H](C)CN(c1ccc(C(=O)N3CCC4(CCN(C)C4)CC3)cc1)C2] | (S)-N-(4-(3,3- dimethylureido)benzyl)- 1-isopropyl-7-methyl-5- (4-(2-methyl-2,8- diazaspiro[4.5]decane- 8-carbonyl)phenyl)- 4,5,6,7-tetrahydro-1H- pyrazolo[4,3-c]pyridine- 3-carboxamide | 655.5 |
| E79A | Example 193^{b} | 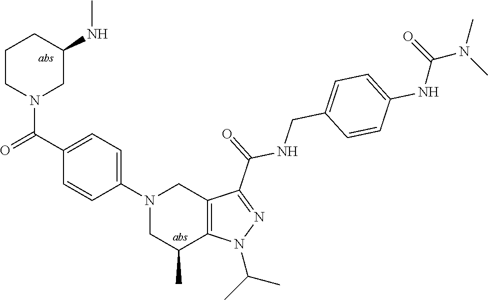 [SMILES: CN[C@@H]1CCCN(C(=O)c2ccc(N3Cc4c(C(=O)NCc5ccc(NC(=O)N(C)C)cc5)nn(C(C)C)c4[C@@H](C)C3)cc2)C1] | (S)-N-(4-(3,3- dimethylureido)benzyl)- 1-isopropyl-7-methyl-5- (4-((R)-3- (methylamino)piperidine- 1-carbonyl)phenyl)- 4,5,6,7-tetrahydro-1H- pyrazolo[4,3-c]pyridine- 3-carboxamide | 655.5 |
| E80A | Example 193^{b} | 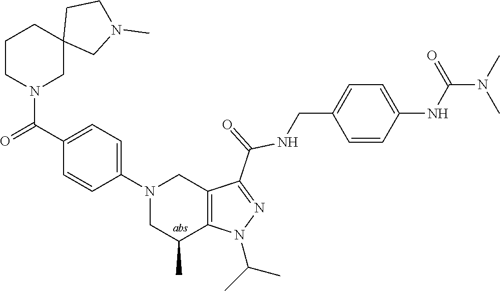 [SMILES: CC(C)n1nc(C(=O)NCc2ccc(NC(=O)N(C)C)cc2)c2c1[C@@H](C)CN(c1ccc(C(=O)N3CCCC4(CCN(C)C4)C3)cc1)C2] | (7S)-N-(4-(3,3- dimethylureido)benzyl)- 1-isopropyl-7-methyl-5- (4-(2-methyl-2,7- diazaspiro[4.5]decane- 7-carbonyl)phenyl)- 4,5,6,7-tetrahydro-1H- pyrazolo[4,3-c]pyridine- 3-carboxamide | 655.5 |
| E81A | Example 193^{ab} | 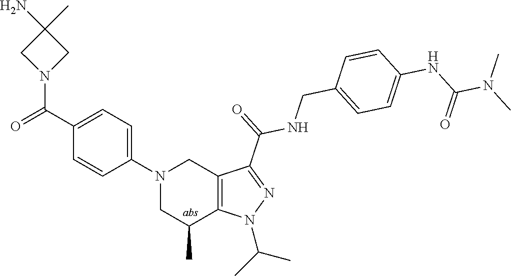 [SMILES: CC(C)n1nc(C(=O)NCc2ccc(NC(=O)N(C)C)cc2)c2c1[C@@H](C)CN(c1ccc(C(=O)N3CC(C)(N)C3)cc1)C2] | (S)-5-(4-(3-amino-3- methylazetidine-1- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 587.5 |
| E82A | Example 193^{b} | 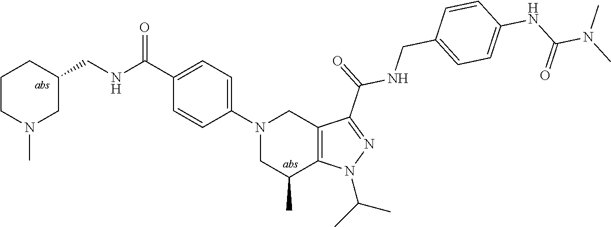 [SMILES: CC(C)n1nc(C(=O)NCc2ccc(NC(=O)N(C)C)cc2)c2c1[C@@H](C)CN(c1ccc(C(=O)NC[C@H]3CCCN(C)C3)cc1)C2] | (S)-N-(4-(3,3- dimethylureido)benzyl)- 1-isopropyl-7-methyl-5- (4-((((R)-1- methylpiperidin-3- yl)methyl)carbamoyl) phenyl)-4,5,6,7-tetrahydro-1H- pyrazolo[4,3-c]pyridine- 3-carboxamide | 629.5 |
| E83A | Example 193^{ab} | 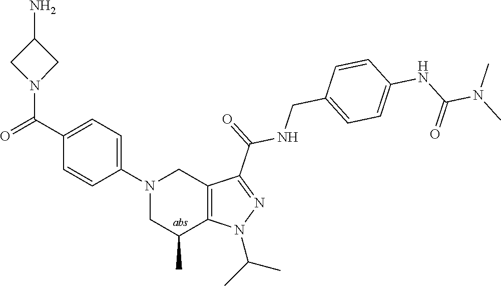 [SMILES: CC(C)n1nc(C(=O)NCc2ccc(NC(=O)N(C)C)cc2)c2c1[C@@H](C)CN(c1ccc(C(=O)N3CC(N)C3)cc1)C2] | (S)-5-(4-(3- aminoazetidine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 573.4 |
| E84A | Example 193^{b} | 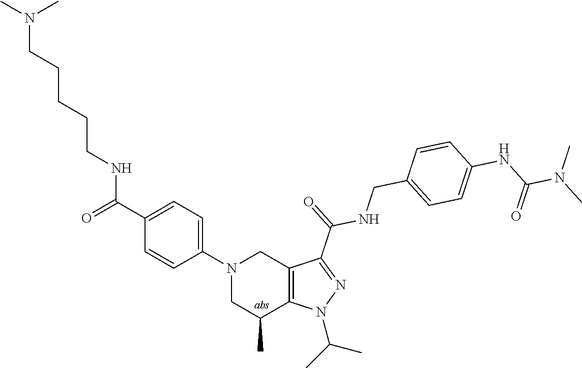 | (S)-5-(4-((5- (dimethylamino)pentyl) carbamoyl)phenyl)-N- (4-(3,3- dimethylureido)benzyl)- 1-isopropyl-7-methyl- 4,5,6,7-tetrahydro-1H- pyrazolo[4,3-c]pyridine- 3-carboxamide | 631.5 |
| E85A | Example 193^{b} | 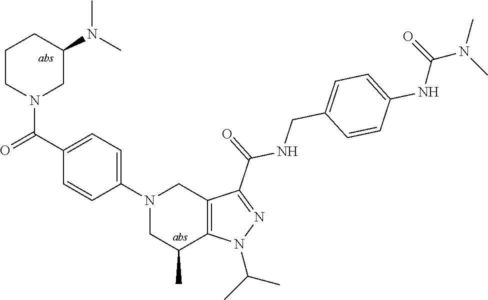 [SMILES: CC(C)n1nc(C(=O)NCc2ccc(NC(=O)N(C)C)cc2)c2c1[C@@H](C)CN(c1ccc(C(=O)N3CCC[C@@H](N(C)C)C3)cc1)C2] | (S)-5-(4-((R)-3- (dimethylamino)piperidine-1- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 629.5 |
| E86A | Example 193^{b} | 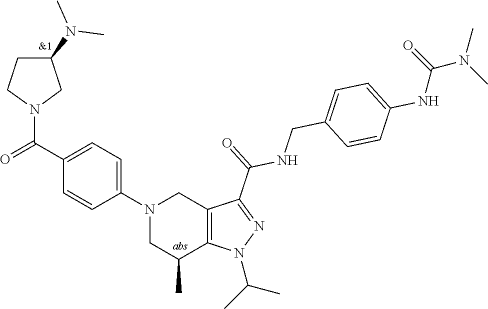 [SMILES: CC(C)n1nc(C(=O)NCc2ccc(NC(=O)N(C)C)cc2)c2c1[C@@H](C)CN(c1ccc(C(=O)N3CC[C@@H](N(C)C)C3)cc1)C2] | (S)-5-(4-((RS)-3- (dimethylamino)pyrrolidine-1- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 615.5 |
| E87A | Example 193^{b} | 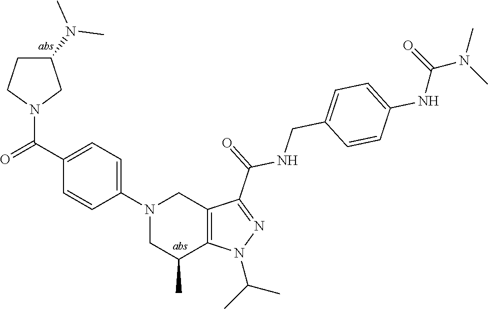 [SMILES: CC(C)n1nc(C(=O)NCc2ccc(NC(=O)N(C)C)cc2)c2c1[C@@H](C)CN(c1ccc(C(=O)N3CC[C@H](N(C)C)C3)cc1)C2] | (S)-5-(4-((S)-3- (dimethylamino)pyrrolidine- 1-carbonyl)phenyl)-N-(4- (3,3-dimethylureido)benzyl)- 1-isopropyl-7-methyl- 4,5,6,7-tetrahydro-1H- pyrazolo[4,3-c]pyridine- 3-carboxamide | 615.5 |
| E88A | Example 193^{b} | 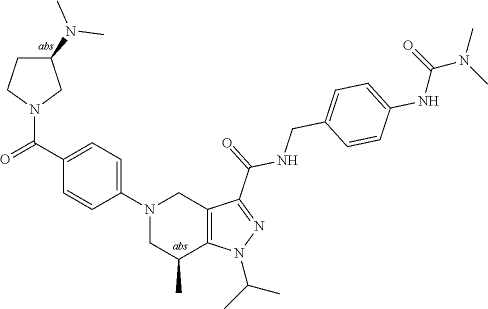 [SMILES: CC(C)n1nc(C(=O)NCc2ccc(NC(=O)N(C)C)cc2)c2c1[C@@H](C)CN(c1ccc(C(=O)N3CC[C@@H](N(C)C)C3)cc1)C2] | (S)-5-(4-((R)-3- (dimethylamino)pyrrolidine-1- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 615.5 |
| E89A | Example 193^{b} | 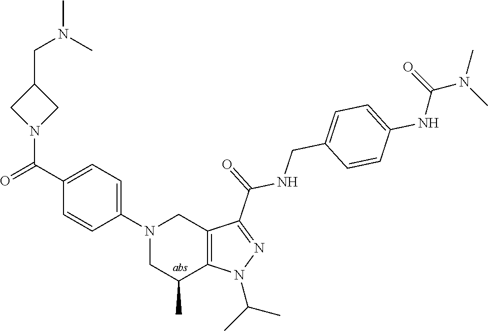 [SMILES: CC(C)n1nc(C(=O)NCc2ccc(NC(=O)N(C)C)cc2)c2c1[C@@H](C)CN(c1ccc(C(=O)N3CC(CN(C)C)C3)cc1)C2] | (S)-5-(4-(3- ((dimethylamino)methyl) azetidine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 615.5 |
| E90A | Example 193^{b} | 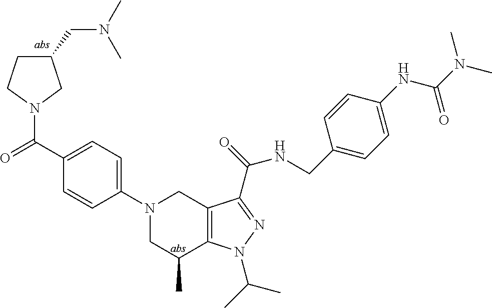 [SMILES: CC(C)n1nc(C(=O)NCc2ccc(NC(=O)N(C)C)cc2)c2c1[C@@H](C)CN(c1ccc(C(=O)N3CC[C@H](CN(C)C)C3)cc1)C2] | (S)-5-(4-((R)-3- ((dimethylamino)methyl) pyrrolidine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 629.5 |
| E91A | Example 193^{b} | 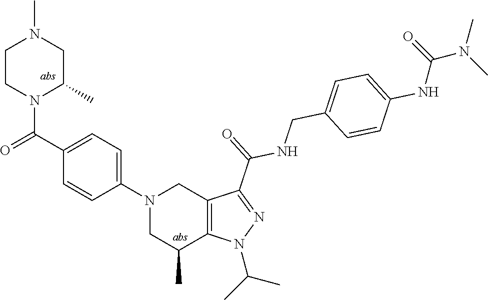 [SMILES: CC(C)n1nc(C(=O)NCc2ccc(NC(=O)N(C)C)cc2)c2c1[C@@H](C)CN(c1ccc(C(=O)N3CCN(C)C[C@@H]3C)cc1)C2] | (S)-5-(4-((S)-2,4- dimethylpiperazine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 615.5 |
| E92A | Example 193^{b} | 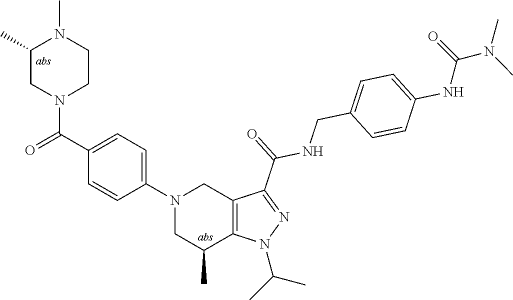 | (S)-5-(4-((S)-3,4- dimethylpiperazine-1- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 615.5 |
| E93A | Example 193^{b} | 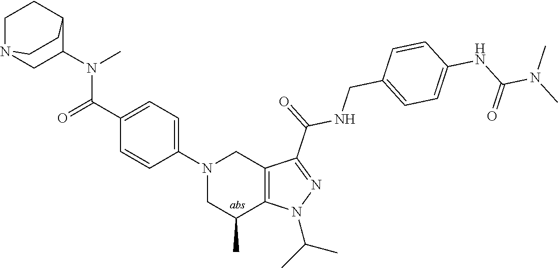 [SMILES: CC(C)n1nc(C(=O)NCc2ccc(NC(=O)N(C)C)cc2)c2c1[C@@H](C)CN(c1ccc(C(=O)N(C)C3CN4CCC3CC4)cc1)C2] | (7S)-N-(4-(3,3- dimethylureido)benzyl)- 1-isopropyl-7-methyl-5- (4-(methyl(quinuclidin- 3-yl)carbamoyl)phenyl)- 4,5,6,7-tetrahydro-1H- pyrazolo[4,3-c]pyridine- 3-carboxamide | 641.5 |
| E94A | Example 193^{b} | 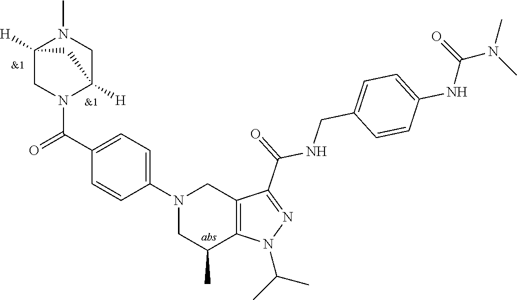 [SMILES: CC(C)n1nc(C(=O)NCc2ccc(NC(=O)N(C)C)cc2)c2c1[C@@H](C)CN(c1ccc(C(=O)N3C[C@@H]4C[C@H]3CN4C)cc1)C2] | (S)-N-(4-(3,3- dimethylureido)benzyl)- 1-isopropyl-7-methyl-5- (4-((1RS,4RS)-5- methyl-2,5- diazabicyclo[2.2.1]heptane-2- carbonyl)phenyl)- 4,5,6,7-tetrahydro-1H- pyrazolo[4,3-c]pyridine- 3-carboxamide | 613.5 |
| E95A | Example 193^{b} | 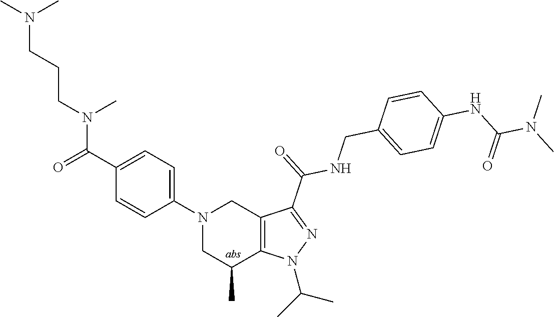 | (S)-5-(4-((3- (dimethylamino)propyl) (methyl)carbamoyl)phenyl)- N-(4-(3,3- dimethylureido)benzyl)- 1-isopropyl-7-methyl- 4,5,6,7-tetrahydro-1H- pyrazolo[4,3-c]pyridine- 3-carboxamide | 617.5 |
| E96A | Example 193^{b} | 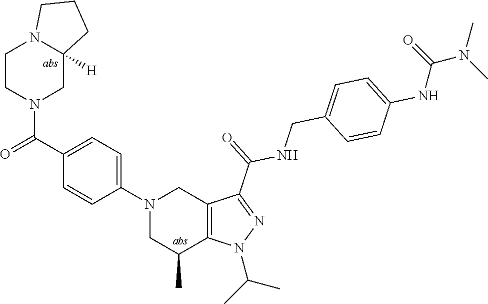 [SMILES: CC(C)n1nc(C(=O)NCc2ccc(NC(=O)N(C)C)cc2)c2c1[C@@H](C)CN(c1ccc(C(=O)N3CCN4CCC[C@H]4C3)cc1)C2] | (S)-N-(4-(3,3- dimethylureido)benzyl)- 1-isopropyl-7-methyl-5- (4-((S)- octahydropyrrolo[1,2- a]pyrazine-2- carbonyl)phenyl)- 4,5,6,7-tetrahydro-1H- pyrazolo[4,3-c]pyridine- 3-carboxamide | 627.5 |
| E97A | Example 193^{b} | 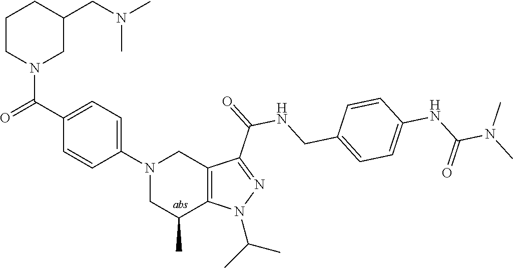 [SMILES: CC(C)n1nc(C(=O)NCc2ccc(NC(=O)N(C)C)cc2)c2c1[C@@H](C)CN(c1ccc(C(=O)N3CCCC(CN(C)C)C3)cc1)C2] | (7S)-5-(4-(3- ((dimethylamino)methyl) piperidine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 643.5 |
| E98A | Example 193^{b} | 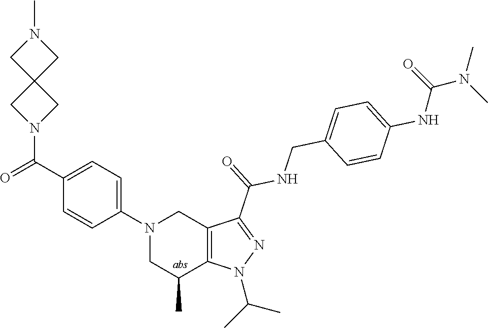 [SMILES: CC(C)n1nc(C(=O)NCc2ccc(NC(=O)N(C)C)cc2)c2c1[C@@H](C)CN(c1ccc(C(=O)N3CC4(CN(C)C4)C3)cc1)C2] | (S)-N-(4-(3,3- dimethylureido)benzyl)- 1-isopropyl-7-methyl-5- (4-(6-methyl-2,6- diazaspiro[3.3]heptane- 2-carbonyl)phenyl)- 4,5,6,7-tetrahydro-1H- pyrazolo[4,3-c]pyridine- 3-carboxamide | 613.5 |
| E99A | Example 193^{b} | 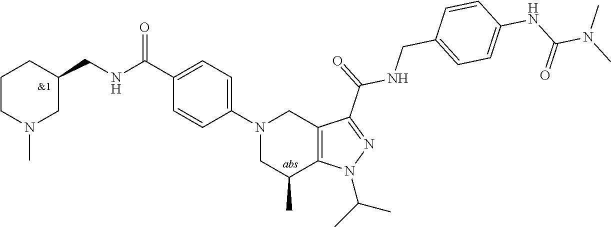 [SMILES: CC(C)n1nc(C(=O)NCc2ccc(NC(=O)N(C)C)cc2)c2c1[C@@H](C)CN(c1ccc(C(=O)NC[C@@H]3CCCN(C)C3)cc1)C2] | (S)-N-(4-(3,3- dimethylureido)benzyl)- 1-isopropyl-7-methyl-5- (4-((((RS)-1- methylpiperidin-3- yl)methyl)carbamoyl) phenyl)-4,5,6,7-tetrahydro-1H- pyrazolo[4,3-c]pyridine- 3-carboxamide | 629.5 |
| E100A | Example 193^{b} | 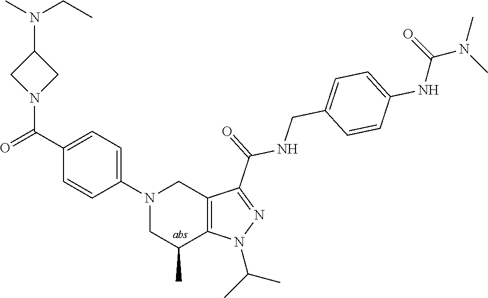 [SMILES: CCN(C)C1CN(C(=O)c2ccc(N3Cc4c(C(=O)NCc5ccc(NC(=O)N(C)C)cc5)nn(C(C)C)c4[C@@H](C)C3)cc2)C1] | (S)-N-(4-(3,3- dimethylureido)benzyl)- 5-(4-(3- (ethyl(methyl)amino) azetidine-1- carbonyl)phenyl)-1- isopropyl-7-methyl- 4,5,6,7-tetrahydro-1H- pyrazolo[4,3-c]pyridine- 3-carboxamide | 615.5 |
| E101A | Example 193^{b} | 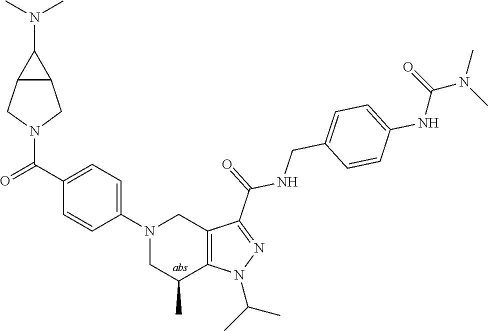 [SMILES: CC(C)n1nc(C(=O)NCc2ccc(NC(=O)N(C)C)cc2)c2c1[C@@H](C)CN(c1ccc(C(=O)N3CC4C(C3)C4N(C)C)cc1)C2] | (7S)-5-(4-(6- (dimethylamino)-3- azabicyclo[3.1.0]hexane-3- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 627.5 |
| E102A | Example 193^{b} | 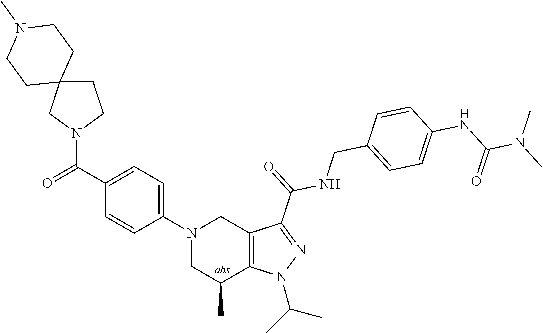 | (S)-N-(4-(3,3- dimethylureido)benzyl)- 1-isopropyl-7-methyl-5- (4-(8-methyl-2,8- diazaspiro[4.5]decane- 2-carbonyl)phenyl)- 4,5,6,7-tetrahydro-1H- pyrazolo[4,3-c]pyridine- 3-carboxamide | 655.5 |
| E103A | Example 193^{b} | 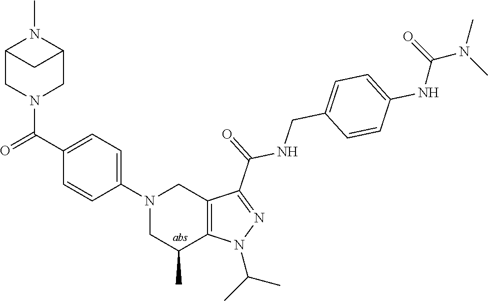 [SMILES: CC(C)n1nc(C(=O)NCc2ccc(NC(=O)N(C)C)cc2)c2c1[C@@H](C)CN(c1ccc(C(=O)N3CC4CC(C3)N4C)cc1)C2] | (7S)-N-(4-(3,3- dimethylureido)benzyl)- 1-isopropyl-7-methyl-5- (4-(6-methyl-3,6- diazabicyclo[3.1.1]heptane- 3-carbonyl)phenyl)- 4,5,6,7-tetrahydro-1H- pyrazolo[4,3-c]pyridine- 3-carboxamide | 613.5 |
| E104A | Example 193^{b} | 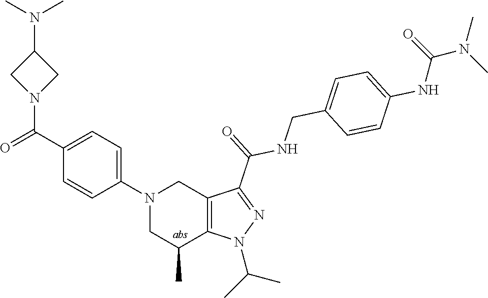 [SMILES: CC(C)n1nc(C(=O)NCc2ccc(NC(=O)N(C)C)cc2)c2c1[C@@H](C)CN(c1ccc(C(=O)N3CC(N(C)C)C3)cc1)C2] | (S)-5-(4-(3- (dimethylamino)azetidine-1- carbonyl)phenyl)- N-(4-(3,3- dimethylureido)benzyl)- 1-isopropyl-7-methyl- 4,5,6,7-tetrahydro-1H- pyrazolo[4,3-c]pyridine- 3-carboxamide | 601.5 |
| E105A | Example 193^{b} | 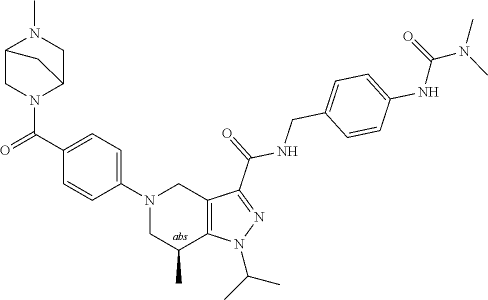 [SMILES: CC(C)n1nc(C(=O)NCc2ccc(NC(=O)N(C)C)cc2)c2c1[C@@H](C)CN(c1ccc(C(=O)N3CC4CC3CN4C)cc1)C2] | (7S)-N-(4-(3,3- dimethylureido)benzyl)- 1-isopropyl-7-methyl-5- (4-(5-methyl-2,5- diazabicyclo[2.2.1]heptane- 2-carbonyl)phenyl)- 4,5,6,7-tetrahydro-1H- pyrazolo[4,3-c]pyridine- 3-carboxamide | 613.5 |
| E106A | Example 193^{b} | 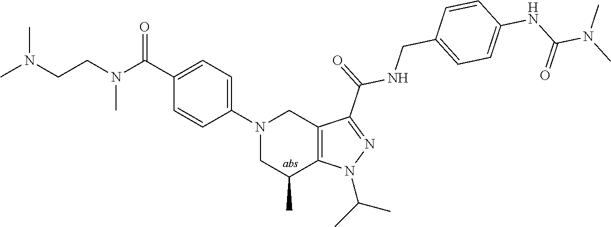 | (S)-5-(4-((2- (dimethylamino)ethyl) (methyl)carbamoyl)phenyl)- N-(4-(3,3- dimethylureido)benzyl)- 1-isopropyl-7-methyl- 4,5,6,7-tetrahydro-1H- pyrazolo[4,3-c]pyridine- 3-carboxamide | 603.5 |
| E107A | Example 193^{b} | 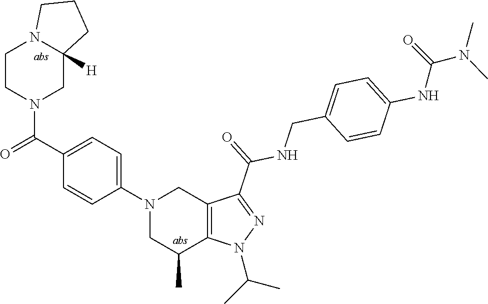 [SMILES: CC(C)n1nc(C(=O)NCc2ccc(NC(=O)N(C)C)cc2)c2c1[C@@H](C)CN(c1ccc(C(=O)N3CCN4CCC[C@@H]4C3)cc1)C2] | (S)-N-(4-(3,3- dimethylureido)benzyl)- 1-isopropyl-7-methyl-5- (4-((R)- octahydropyrrolo[1,2- a]pyrazine-2- carbonyl)phenyl)- 4,5,6,7-tetrahydro-1H- pyrazolo[4,3-c]pyridine- 3-carboxamide | 627.5 |
| E108A | Example 193^{b} | 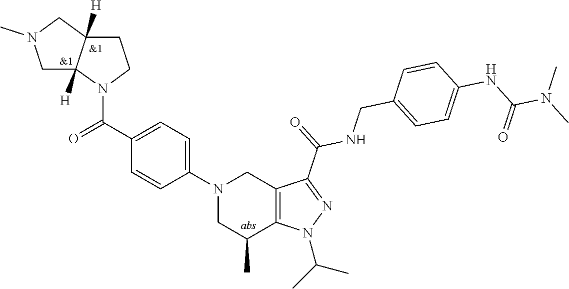 | (S)-N-(4-(3,3- dimethylureido)benzyl)- 1-isopropyl-7-methyl-5- (4-((3aSR,6aSR)-5- methyloctahydropyrrolo [3,4-b]pyrrole-1- carbonyl)phenyl)- 4,5,6,7-tetrahydro-1H- pyrazolo[4,3-c]pyridine- 3-carboxamide | 627.5 |
| E109A | Example 193^{b} | 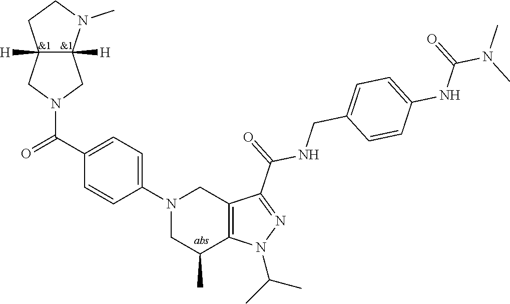 [SMILES: CC(C)n1nc(C(=O)NCc2ccc(NC(=O)N(C)C)cc2)c2c1[C@@H](C)CN(c1ccc(C(=O)N3C[C@@H]4CCN(C)[C@@H]4C3)cc1)C2] | (S)-N-(4-(3,3- dimethylureido)benzyl)- 1-isopropyl-7-methyl-5- (4-((3aSR,6aSR)-1- methyloctahydropyrrolo [3,4-b]pyrrole-5- carbonyl)phenyl)- 4,5,6,7-tetrahydro-1H- pyrazolo[4,3-c]pyridine- 3-carboxamide | 627.5 |
| E110A | Example 193^{b} | 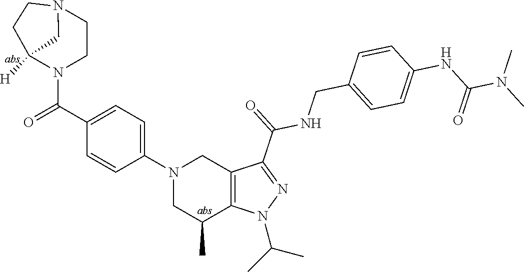 [SMILES: CC(C)n1nc(C(=O)NCc2ccc(NC(=O)N(C)C)cc2)c2c1[C@@H](C)CN(c1ccc(C(=O)N3CCN4CC[C@H]3C4)cc1)C2] | (7S)-5-(4-((5S)-1,4- diazabicyclo[3.2.1]octane-4- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 613.5 |
| E111A | Example 193^{b} | 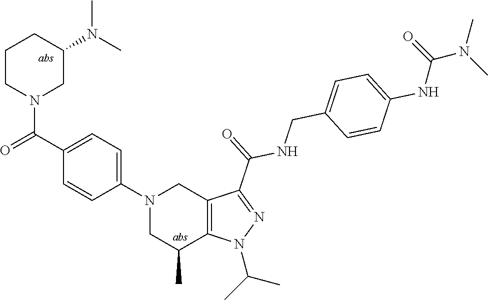 [SMILES: CC(C)n1nc(C(=O)NCc2ccc(NC(=O)N(C)C)cc2)c2c1[C@@H](C)CN(c1ccc(C(=O)N3CCC[C@H](N(C)C)C3)cc1)C2] | (S)-5-(4-((S)-3- (dimethylamino)piperidine- 1-carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 629.5 |
| E112A | Example 193^{b} | 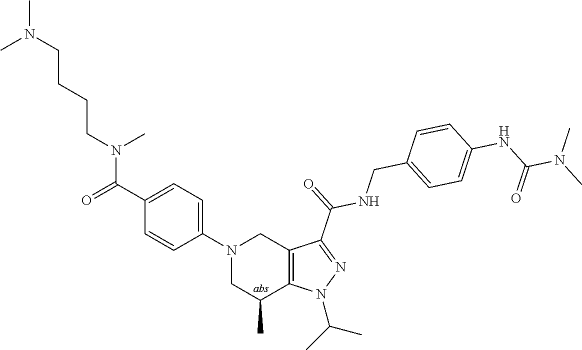 | (S)-5-(4-((4- (dimethylamino)butyl) (methyl)carbamoyl) phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 631.5 |
| E113A | Example 193^{b} | 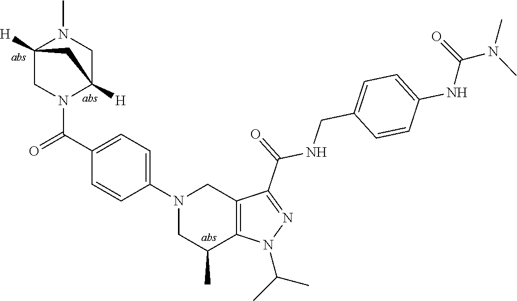 [SMILES: CC(C)n1nc(C(=O)NCc2ccc(NC(=O)N(C)C)cc2)c2c1[C@@H](C)CN(c1ccc(C(=O)N3C[C@H]4C[C@@H]3CN4C)cc1)C2] | (S)-N-(4-(3,3- dimethylureido)benzyl)- 1-isopropyl-7-methyl-5- (4-((1R,4R)-5-methyl- 2,5- diazbicyclo[2.2.1]heptane-2- carbonyl)phenyl)- 4,5,6,7-tetrahydro-1H- pyrazolo[4,3-c]pyridine- 3-carboxamide | 613.5 |
| E114A | Example 193^{b} | 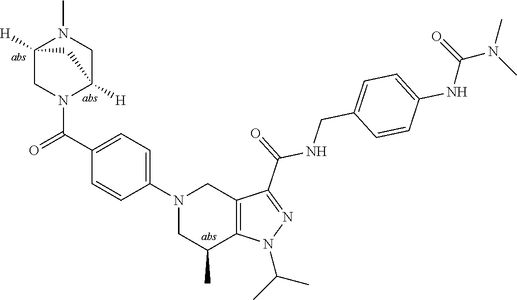 [SMILES: CC(C)n1nc(C(=O)NCc2ccc(NC(=O)N(C)C)cc2)c2c1[C@@H](C)CN(c1ccc(C(=O)N3C[C@@H]4C[C@H]3CN4C)cc1)C2] | (S)-N-(4-(3,3- dimethylureido)benzyl)- 1-isopropyl-7-methyl-5- (4-((1S,4S)-5-methyl- 2,5- diazabicyclo[2.2.1]heptane-2- carbonyl)phenyl)- 4,5,6,7-tetrahydro-1H- pyrazolo[4,3-c]pyridine- 3-carboxamide | 613.5 |
| E115A | Example 193^{b} | 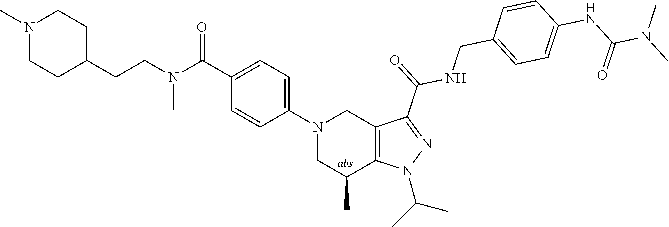 | (S)-N-(4-(3,3- dimethylureido)benzyl)- 1-isopropyl-7-methyl-5- (4-(methyl(2-(1- methylpiperidin-4- yl)ethyl)carbamoyl) phenyl)-4,5,6,7-tetrahydro-1H- pyrazolo[4,3-c]pyridine- 3-carboxamide | 657.6 |
| E116A | Example 193^{b} | 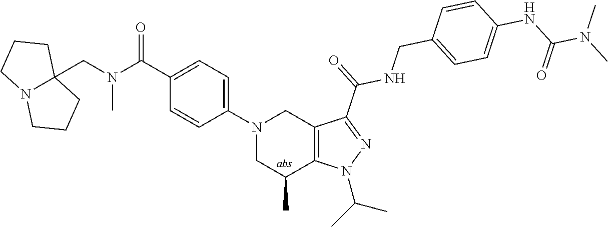 [SMILES: CC(C)n1nc(C(=O)NCc2ccc(NC(=O)N(C)C)cc2)c2c1[C@@H](C)CN(c1ccc(C(=O)N(C)CC34CCCN3CCC4)cc1)C2] | (S)-N-(4-(3,3- dimethylureido)benzyl)- 1-isopropyl-7-methyl-5- (4-(methyl((tetrahydro- 1H-pyrrolizin-7a(5H)- yl)methyl)carbamoyl) phenyl)-4,5,6,7-tetrahydro-1H- pyrazolo[4,3-c]pyridine- 3-carboxamide | 655.6 |
| E117A | Example 193^{b} | 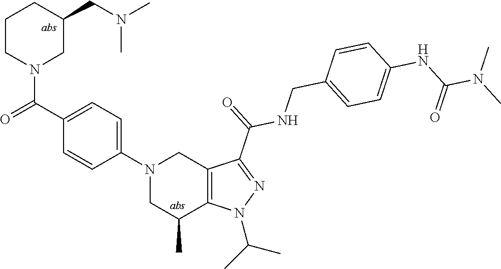 [SMILES: CC(C)n1nc(C(=O)NCc2ccc(NC(=O)N(C)C)cc2)c2c1[C@@H](C)CN(c1ccc(C(=O)N3CCC[C@@H](CN(C)C)C3)cc1)C2] | (S)-5-(4-((S)-3- ((dimethylamino)methyl) piperidine-1- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 643.5 |
| E118A | Example 193^{b} | 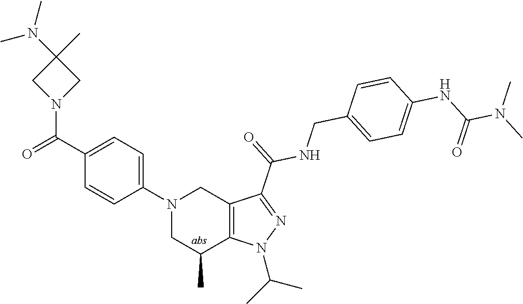 | (S)-5-(4-(3- (dimethylamino)-3- methylazetidine-1- carbonyl)phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 615.5 |
| E119A | Example 193^{b} | 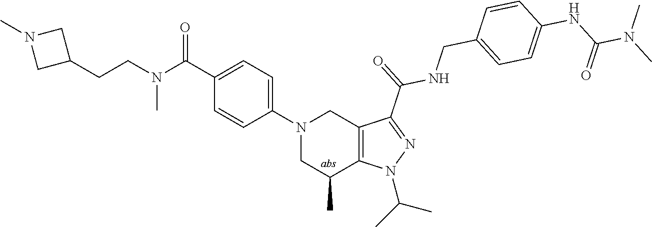 | (S)-N-(4-(3,3- dimethylureido)benzyl)- 1-isopropyl-7-methyl-5- (4-(methyl(2-(1- methylazetidin-3- yl)ethyl)carbamoyl) phenyl)-4,5,6,7-tetrahydro-1H- pyrazolo[4,3-c]pyridine- 3-carboxamide | 629.5 |
| E120A | Example 193^{b} | 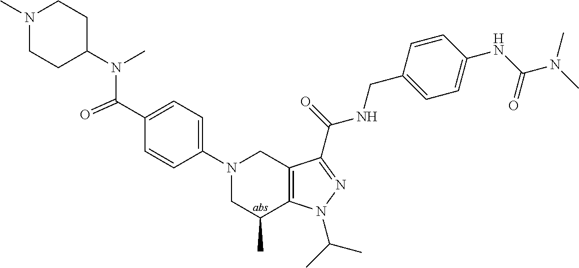 | (S)-N-(4-(3,3- dimethylureido)benzyl)- 1-isopropyl-7-methyl-5- (4-(methyl(1- methylpiperidin-4- yl)carbamoyl)phenyl)- 4,5,6,7-tetrahydro-1H- pyrazolo[4,3-c]pyridine- 3-carboxamide | 629.5 |
| E121A | Example 193^{b} | 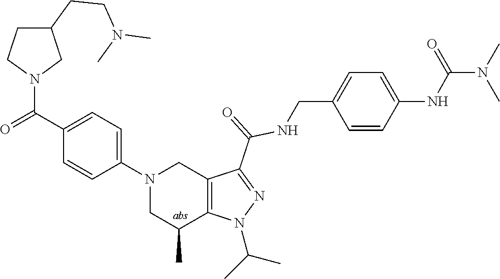 [SMILES: CC(C)n1nc(C(=O)NCc2ccc(NC(=O)N(C)C)cc2)c2c1[C@@H](C)CN(c1ccc(C(=O)N3CCC(CCN(C)C)C3)cc1)C2] | (7S)-5-(4-(3-(2- (dimethylamino)ethyl) pyrrolidine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 643.5 |
| E122A | Example 193^{b} | 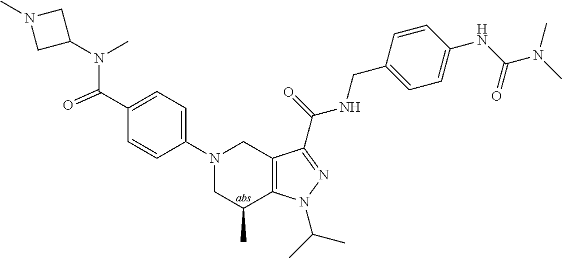 | (S)-N-(4-(3,3- dimethylureido)benzyl)- 1-isopropyl-7-methyl-5- (4-(methyl(1- methylazetidin-3- yl)carbamoyl)phenyl)- 4,5,6,7-tetrahydro-1H- pyrazolo[4,3-c]pyridine- 3-carboxamide | 601.5 |
| E123A | Example 193^{b} | 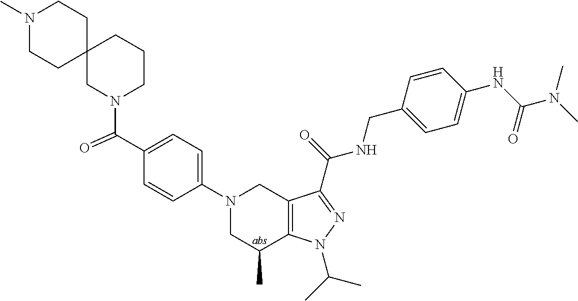 | (S)-N-(4-(3,3- dimethylureido)benzyl)- 1-isopropyl-7-methyl-5- (4-(9-methyl-2,9- diazaspiro[5.5]undecane-2- carbonyl)phenyl)- 4,5,6,7-tetrahydro-1H- pyrazolo[4,3-c]pyridine- 3-carboxamide | 699.5 |
| E124A | Example 193^{b} | 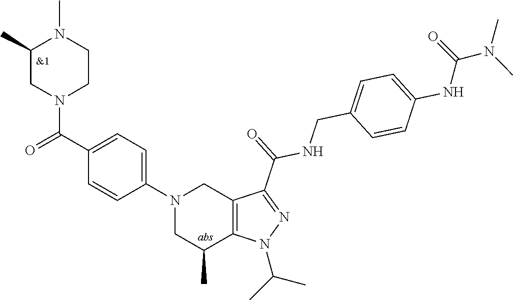 | (S)-5-(4-((RS)-3,4- dimethylpiperazine-1- carbonyl)phenyl)-N-(4- (3,3- dimethylureido)benzyl)- 1-isopropyl-7-methyl- 4,5,6,7-tetrahydro-1H- pyrazolo[4,3-c]pyridine- 3-carboxamide | 615.5 |
| E125A | Example 193^{b} | 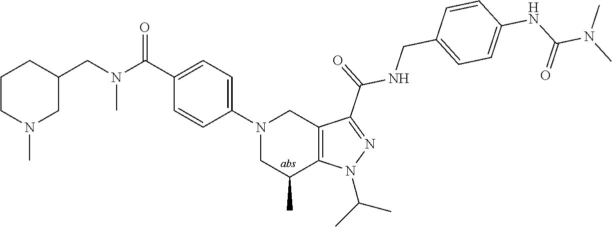 [SMILES: CC(C)n1nc(C(=O)NCc2ccc(NC(=O)N(C)C)cc2)c2c1[C@@H](C)CN(c1ccc(C(=O)N(C)CC3CCCN(C)C3)cc1)C2] | (7S)-N-(4-(3,3- dimethylureido)benzyl)- 1-isopropyl-7-methyl-5- (4-(methyl((1- methylpiperidin-3- yl)methyl)carbamoyl) phenyl)-4,5,6,7-tetrahydro-1H- pyrazolo[4,3-c]pyridine- 3-carboxamide | 643.5 |
| E126A | Example 193^{b} | 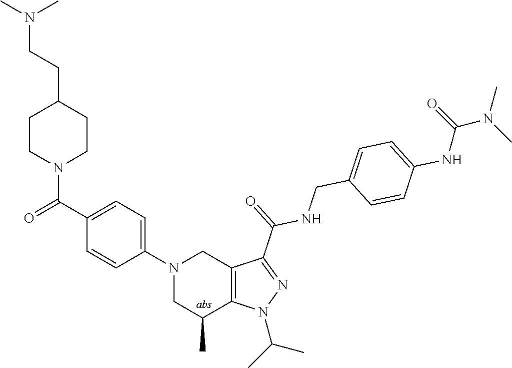 | (S)-5-(4-(4-(2- (dimethylamino)ethyl) piperidine-1-carbonyl) phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 657.5 |
| E127A | Example 193^{b} | 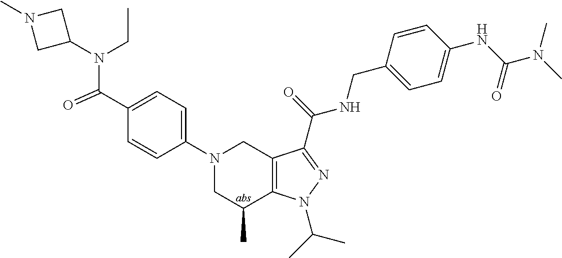 | (S)-N-(4-(3,3- dimethylureido)benzyl)- 5-(4-(ethyl(1- methylazetidin-3- yl)carbamoyl)phenyl)-1- isopropyl-7-methyl- 4,5,6,7-tetrahydro-1H- pyrazolo[4,3-c]pyridine- 3-carboxamide | 615.5 |
| E128A | Example 193^{b} | 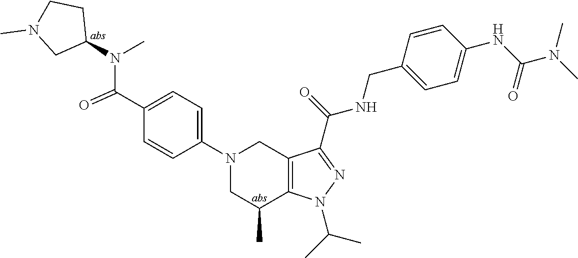 | (S)-N-(4-(3,3- dimethylureido)benzyl)- 1-isopropyl-7-methyl-5- (4-(methyl((R)-1- methylpyrrolidin-3- yl)carbamoyl)phenyl)- 4,5,6,7-tetrahydro-1H- pyrazolo[4,3-c]pyridine- 3-carboxamide | 615.5 |
| E129A | Example 193^{b} | 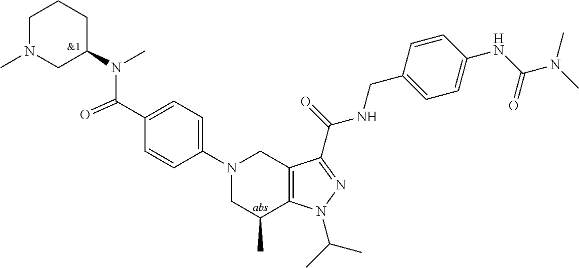 | (S)-N-(4-(3,3- dimethylureido)benzyl)- 1-isopropyl-7-methyl-5- (4-(methyl((RS)-1- methylpiperidin-3- yl)carbamoyl)phenyl)- 4,5,6,7-tetrahydro-1H- pyrazolo[4,3-c]pyridine- 3-carboxamide | 629.6 |
| E130A | Example 193^{b} | 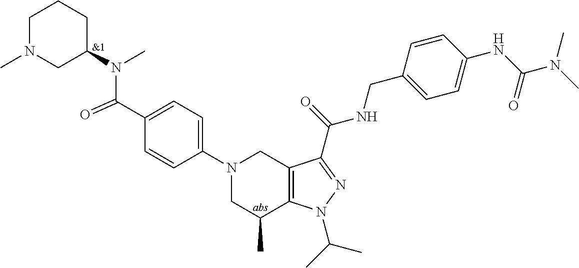 | (S)-N-(4-(3,3- dimethylureido)benzyl)- 1-isopropyl-7-methyl-5- (4-(methyl((RS)-1- methylpyrrolidin-3- yl)carbamoyl)phenyl)- 4,5,6,7-tetrahydro-1H- pyrazolo[4,3-c]pyridine- 3-carboxamide | 615.5 |
| E131A | Example 193^{b} | 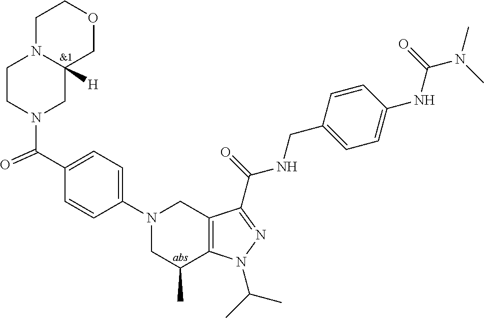 [SMILES: CC(C)n1nc(C(=O)NCc2ccc(NC(=O)N(C)C)cc2)c2c1[C@@H](C)CN(c1ccc(C(=O)N3CCN4CCOC[C@@H]4C3)cc1)C2] | (S)-N-(4-(3,3- dimethylureido)benzyl)- 1-isopropyl-7-methyl-5- (4-((SR)- octahydropyrazino[2,1- c][1,4]oxazine-8- carbonyl)phenyl)- 4,5,6,7-tetrahydro-1H- pyrazolo[4,3-c]pyridine- 3-carboxamide | 643.5 |
| E132A | Example 193^{b} | 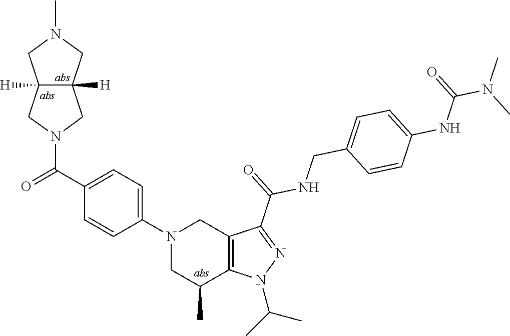 [SMILES: CC(C)n1nc(C(=O)NCc2ccc(NC(=O)N(C)C)cc2)c2c1[C@@H](C)CN(c1ccc(C(=O)N3C[C@H]4CN(C)C[C@@H]4C3)cc1)C2] | (S)-N-(4-(3,3- dimethylureido)benzyl)- 1-isopropyl-7-methyl-5- (4-((3aR,6aR)-5- methyloctahydropyrrolo [3,4-c]pyrrole-2- carbonyl)phenyl)- 4,5,6,7-tetrahydro-1H- pyrazolo[4,3-c]pyridine- 3-carboxamide | 627.5 |
| E133A | Example 127 | 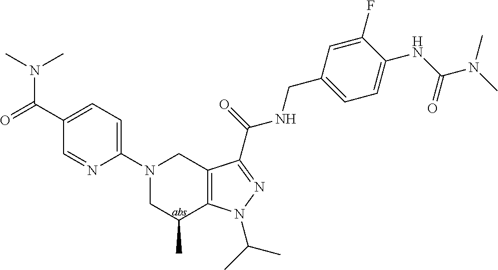 [SMILES: CC(C)n1nc(C(=O)NCc2ccc(NC(=O)N(C)C)c(F)c2)c2c1[C@@H](C)CN(c1ccc(C(=O)N(C)C)cn1)C2] | (S)-5-(5- (dimethylcarbamoyl) pyridin-2-yl)-N-(4-(3,3- dimethylureido)-3- fluorobenzyl)-1- isopropyl-7-methyl- 4,5,6,7-tetrahydro-1H- pyrazolo[4,3-c]pyridine- 3-carboxamide | 565.4 |
| E134A | Example 126^{b} | 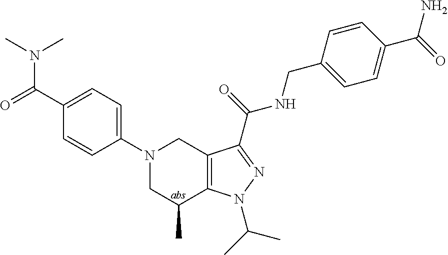 [SMILES: CC(C)n1nc(C(=O)NCc2ccc(C(N)=O)cc2)c2c1[C@@H](C)CN(c1ccc(C(=O)N(C)C)cc1)C2] | (S)-N-(4- carbamoylbenzyl)-5-(4- (dimethylcarbamoyl) phenyl)-1-isopropyl-7- methyl-4,5,6,7-tetrahydro-1H- pyrazolo[4,3-c]pyridine- 3-carboxamide | 503.4 |
| E135A | Example 126^{b} | 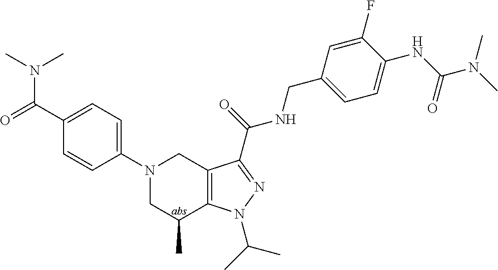 [SMILES: CC(C)n1nc(C(=O)NCc2ccc(NC(=O)N(C)C)c(F)c2)c2c1[C@@H](C)CN(c1ccc(C(=O)N(C)C)cc1)C2] | (S)-5-(4- (dimethylcarbamoyl) phenyl)-N-(4-(3,3- dimethylureido)-3- fluorobenzyl)-1- isopropyl-7-methyl- 4,5,6,7-tetrahydro-1H- pyrazolo[4,3-c]pyridine- 3-carboxamide | 564.4 |
| E136A | Example 567 | 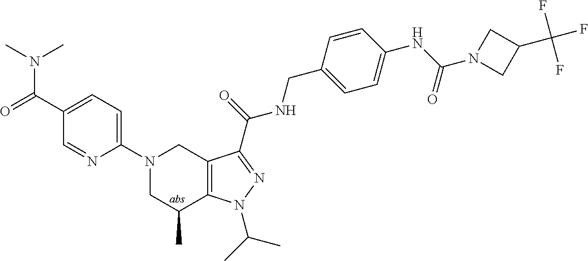 [SMILES: CC(C)n1nc(C(=O)NCc2ccc(NC(=O)N3CC(C(F)(F)F)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(3- (trifluoromethyl)azetidine-1- carboxamido)benzyl)- 4,5,6,7-tetrahydro-1H- pyrazolo[4,3-c]pyridine- 3-carboxamide | 627.4 |
| E137A | Example 126^{b} | 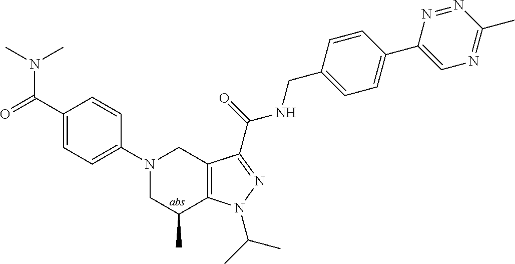 [SMILES: Cc1ncc(-c2ccc(CNC(=O)c3nn(C(C)C)c4c3CN(c3ccc(C(=O)N(C)C)cc3)C[C@@H]4C)cc2)nn1] | (S)-5-(4- (dimethylcarbamoyl) phenyl)-1-isopropyl-7- methyl-N-(4-(3-methyl- 1,2,4-triazin-6- yl)benzyl)-4,5,6,7- tetrahydro-1H- pyrazolo[4,3-c]pyridine- 3-carboxamide | 553.3 |
| E138A | Example 485 | 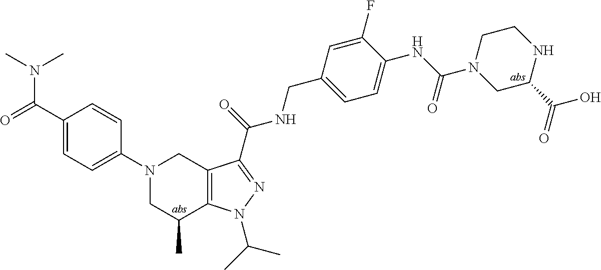 [SMILES: CC(C)n1nc(C(=O)NCc2ccc(NC(=O)N3CCN[C@H](C(=O)O)C3)c(F)c2)c2c1[C@@H](C)CN(c1ccc(C(=O)N(C)C)cc1)C2] | (S)-4-((4-(((S)-5-(4- (dimethylcarbamoyl) phenyl)-1-isopropyl-7- methyl-4,5,6,7- tetrahydro-1H- pyrazolo[4,3-c]pyridine- 3-carboxamido)methyl)-2- fluorophenyl)carbamoyl) piperazine-2-carboxylic acid | 649.3 |
| E139A | Example 567 | 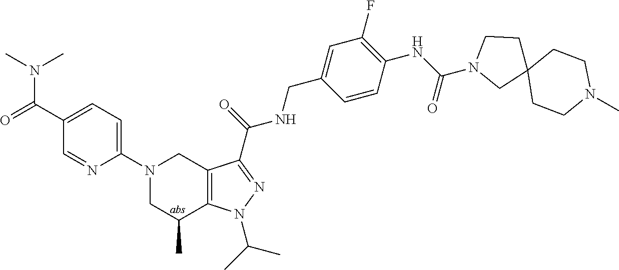 [SMILES: CC(C)n1nc(C(=O)NCc2ccc(NC(=O)N3CCC4(CCN(C)CC4)C3)c(F)c2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7- tetrahydro-1H- pyrazolo[4,3-c]pyridine- 3-carboxamido)methyl)- 2-fluorophenyl)-8- methyl-2,8- diazaspiro[4.5]decane- 2-carboxamide | 674.4 |
| E140A | Example 567 | 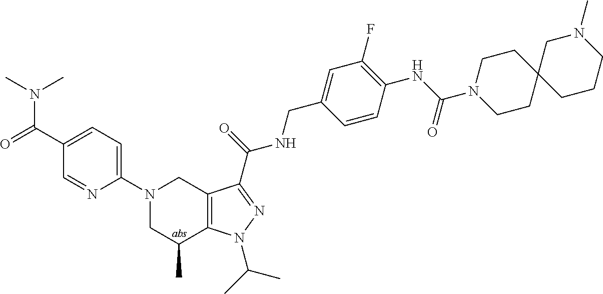 [SMILES: CC(C)n1nc(C(=O)NCc2ccc(NC(=O)N3CCC4(CCCN(C)C4)CC3)c(F)c2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7- tetrahydro-1H- pyrazolo[4,3-c]pyridine- 3-carboxamido)methyl)- 2-fluorophenyl)-2- methyl-2,9-diazaspiro [5.5]undecane-9-carboxamide | 688.4 |
| E141A | Example 567 | 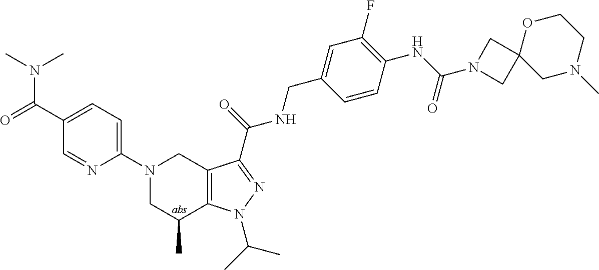 [SMILES: CC(C)n1nc(C(=O)NCc2ccc(NC(=O)N3CC4(CN(C)CCO4)C3)c(F)c2)c2c1[C@@H](C)CN(c1ccc(C(=O)N(C)C)cn1)C2] | (S)-N-(4-((5-(5- (dimethylcarbamoyl)pyridin- 2-yl)-1-isopropyl-7- methyl-4,5,6,7- tetrahydro-1H- pyrazolo[4,3-c]pyridine- 3-carboxamido)methyl)- 2-fluorophenyl)-8- methyl-5-oxa-2,8- diazaspiro[3.5]nonane- 2-carboxamide | 662.4 |
| E142A | Example 127 | 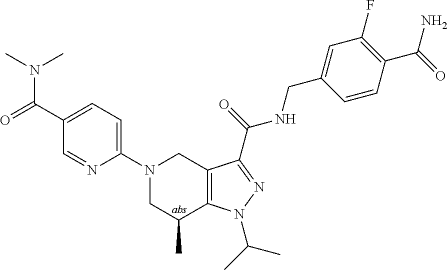 [SMILES: CC(C)n1nc(C(=O)NCc2ccc(C(N)=O)c(F)c2)c2c1[C@@H](C)CN(c1ccc(C(=O)N(C)C)cn1)C2] | (S)-N-(4-carbamoyl-3- fluorobenzyl)-5-(5- (dimethylcarbamoyl) pyridin-2-yl)-1-isopropyl-7- methyl-4,5,6,7- tetrahydro-1H- pyrazolo[4,3-c]pyridine- 3-carboxamide | 522.4 |
| E143A | Example 127 | 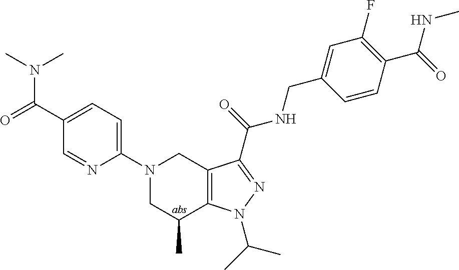 [SMILES: CNC(=O)c1ccc(CNC(=O)c2nn(C(C)C)c3c2CN(c2ccc(C(=O)N(C)C)cn2)C[C@@H]3C)cc1F] | (S)-5-(5- (dimethylcarbamoyl) pyridin-2-yl)-N-(3-fluoro-4- (methylcarbamoyl)benzyl)-1- isopropyl-7-methyl- 4,5,6,7- tetrahydro-1H- pyrazolo[4,3-c]pyridine- 3-carboxamide | 536.4 |
| E144A | Example 126^{b} | 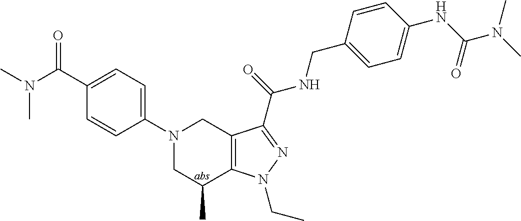 | (S)-5-(4- (dimethylcarbamoyl) phenyl)-N-(4-(3,3- dimethylureido)benzyl)- 1-ethyl-7-methyl- 4,5,6,7- tetrahydro-1H- pyrazolo[4,3-c]pyridine- 3-carboxamide | 532.4 |
| E145A | Example 126 | 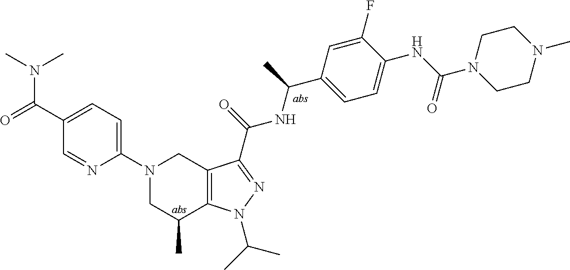 [SMILES: CC(C)n1nc(C(=O)N[C@@H](C)c2ccc(NC(=O)N3CCN(C)CC3)c(F)c2)c2c1[C@@H](C)CN(c1ccc(C(=O)N(C)C)cn1)C2] | (S)-5-(5- (dimethylcarbamoyl) pyridin-2-yl)-N-((S)-1-(3- fluoro-4-(4- methylpiperazine-1- carboxamido)phenyl) ethyl)-1-isopropyl-7- methyl-4,5,6,7- tetrahydro-1H- pyrazolo[4,3-c]pyridine- 3-carboxamide | 634.3 |
| E146A | Example 126 | 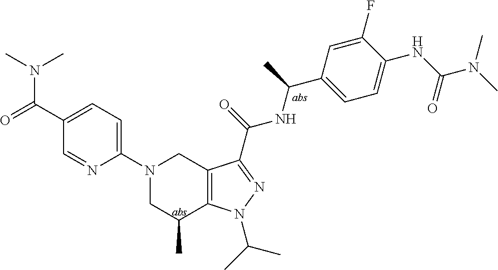 [SMILES: CC(C)n1nc(C(=O)N[C@@H](C)c2ccc(NC(=O)N(C)C)c(F)c2)c2c1[C@@H](C)CN(c1ccc(C(=O)N(C)C)cn1)C2] | (S)-5-(5- (dimethylcarbamoyl) pyridin-2-yl)-N-((S)-1-(4- (3,3-dimethylureido)-3- fluorophenyl)ethyl)-1- isopropyl-7-methyl- 4,5,6,7-tetrahydro-1H- pyrazolo[4,3-c]pyridine- 3-carboxamide | 579.5 |
| E147A | Example 126 | 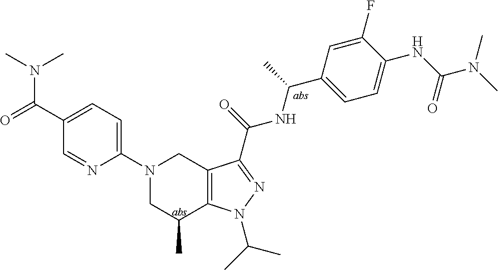 [SMILES: CC(C)n1nc(C(=O)N[C@H](C)c2ccc(NC(=O)N(C)C)c(F)c2)c2c1[C@@H](C)CN(c1ccc(C(=O)N(C)C)cn1)C2] | (S)-5-(5- (dimethylcarbamoyl) pyridin-2-yl)-N-((R)-1-(4- (3,3-dimethylureido)-3- fluorophenyl)ethyl)-1- isopropyl-7-methyl- 4,5,6,7-tetrahydro-1H- pyrazolo[4,3-c]pyridine- 3-carboxamide | 579.4 |
| E148A | Example 127 | 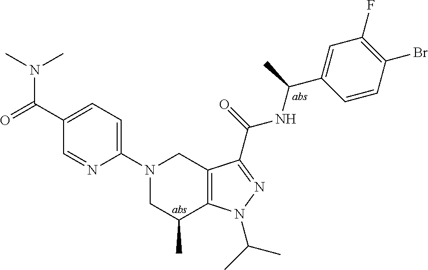 [SMILES: CC(C)n1nc(C(=O)N[C@@H](C)c2ccc(Br)c(F)c2)c2c1[C@@H](C)CN(c1ccc(C(=O)N(C)C)cn1)C2] | (S)-N-((S)-1-(4-bromo- 3-fluorophenyl)ethyl)-5- (5-(dimethylcarbamoyl) pyridin-2-yl)-1-isopropyl-7- methyl- 4,5,6,7-tetrahydro-1H- pyrazolo[4,3-c]pyridine- 3-carboxamide | 571.2 |
| E149A | Example 126^{b} | 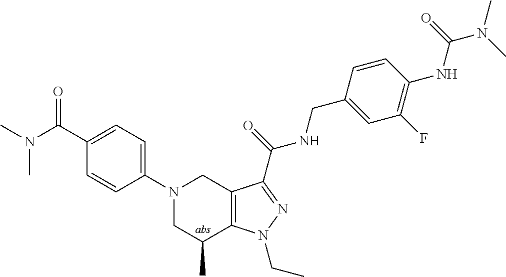 | (S)-5-(4- (dimethylcarbamoyl) phenyl)-N-(4-(3,3- dimethylureido)-3- fluorobenzyl)-1-ethyl-7- 4,5,6,7-tetrahydro-1H- pyrazolo[4,3-c]pyridine- 3-carboxamide | 550.4 |
| E150A | Example 126 | 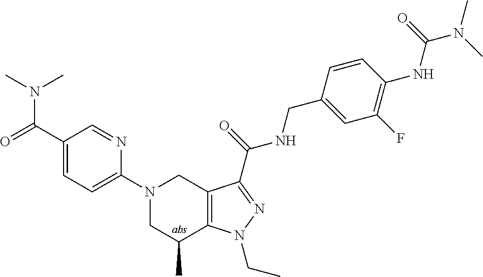 [SMILES: CCn1nc(C(=O)NCc2ccc(NC(=O)N(C)C)c(F)c2)c2c1[C@@H](C)CN(c1ccc(C(=O)N(C)C)cn1)C2] | (S)-5-(5- (dimethylcarbamoyl) pyridin-2-yl)-N-(4-(3,3- dimethylureido)-3- fluorobenzyl)-1-ethyl-7- methyl-4,5,6,7-tetrahydro-1H- pyrazolo[4,3-c]pyridine- 3-carboxamide | 551.4 |
| E151A | Example 567 | 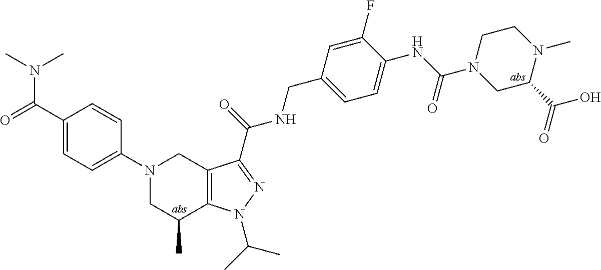 [SMILES: CC(C)n1nc(C(=O)NCc2ccc(NC(=O)N3CCN(C)[C@H](C(=O)O)C3)c(F)c2)c2c1[C@@H](C)CN(c1ccc(C(=O)N(C)C)cc1)C2] | (S)-4-((4-(((S)-5-(4- (dimethylcarbamoyl) phenyl)-1-isopropyl-7- methyl-4,5,6,7-tetrahydro-1H- pyrazolo[4,3-c]pyridine- 3-carboxamido)methyl)-2- fluorophenyl)carbamoyl)-1- methylpiperazine-2- carboxylic acid | 663.5 |
| E152A | Example 127^{b} | 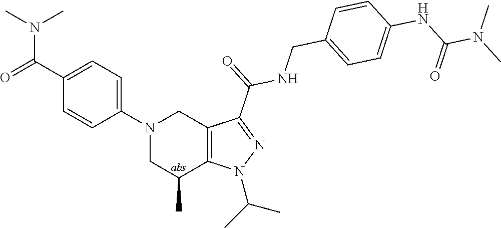 [SMILES: CC(C)n1nc(C(=O)NCc2ccc(NC(=O)N(C)C)cc2)c2c1[C@@H](C)CN(c1ccc(C(=O)N(C)C)cc1)C2] | (S)-5-(4- (dimethylcarbamoyl) phenyl)-N-(4-(3,3- dimethylureido)benzyl)- 1-isopropyl-7-methyl- 4,5,6,7-tetrahydro-1H- pyrazolo[4,3-c]pyridine- 3-carboxamide | 546.4 |
| E153A | Example 567 | 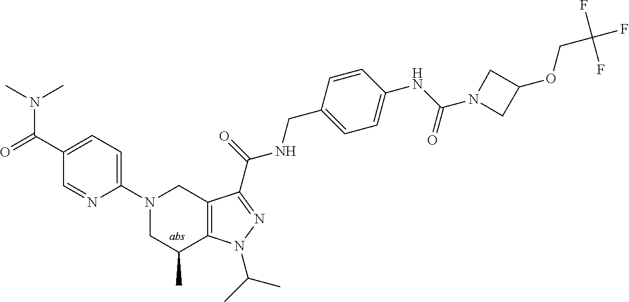 [SMILES: CC(C)n1nc(C(=O)NCc2ccc(NC(=O)N3CC(OCC(F)(F)F)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(3-(2,2,2- trifluoroethoxy)azetidine- 1-carboxamido)benzyl)- 4,5,6,7-tetrahydro-1H- pyrazolo[4,3-c]pyridine- 3-carboxamide | 657.5 |
| E154A | Example 567 | 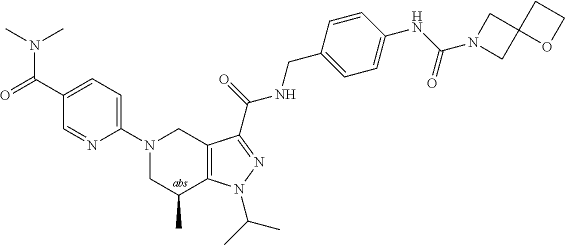 [SMILES: CC(C)n1nc(C(=O)NCc2ccc(NC(=O)N3CC4(CCO4)C3)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-1-oxa-6- azaspiro[3.3]heptane- 6-carboxamide | 601.5 |
| E155A | Example 567 | 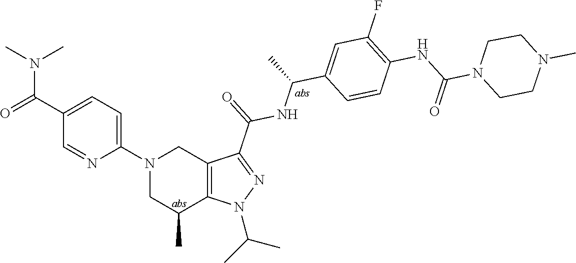 [SMILES: CC(C)n1nc(C(=O)N[C@H](C)c2ccc(NC(=O)N3CCN(C)CC3)c(F)c2)c2c1[C@@H](C)CN(c1ccc(C(=O)N(C)C)cn1)C2] | (S)-5-(5- (dimethylcarbamoyl) pyridin-2-yl)-N-((R)-1-(3- fluoro-4-(4- methylpiperazine-1- carboxamido)phenyl) ethyl)-1-isopropyl-7- methyl-4,5,6,7-tetrahydro-1H- pyrazolo[4,3-c]pyridine- 3-carboxamide | 634.6 |
| E156A | Example 127^{a} | 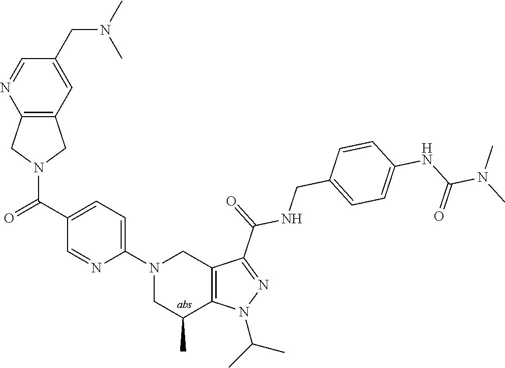 [SMILES: CC(C)n1nc(C(=O)NCc2ccc(NC(=O)N(C)C)cc2)c2c1[C@@H](C)CN(c1ccc(C(=O)N3Cc4cc(CN(C)C)cnc4C3)cn1)C2] | (S)-5-(5-(3- ((dimethylamino)methyl)- 6,7-dihydro-5H-pyrrolo [3,4-b]pyridine-6- carbonyl)pyridin-2- yl)-N-(4-(3,3- dimethylureido)benzyl)- 1-isopropyl-7-methyl- 4,5,6,7-tetrahydro-1H- pyrazolo[4,3-c]pyridine- 3-carboxamide | 679.5 |
| E157A | Example 567 | 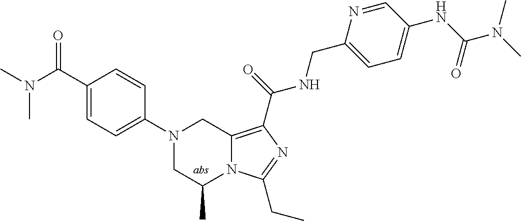 | (S)-5-(4- (dimethylcarbamoyl) phenyl)-N-((5-(3,3- dimethylureido)pyridin- 2-yl)methyl)-1-ethyl-7- methyl-4,5,6,7-tetrahydro-1H- pyrazolo[4,3-c]pyridine- 3-carboxamide | 533.4 |
| E158A | Example 127 | 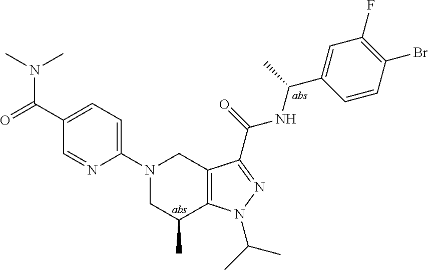 [SMILES: CC(C)n1nc(C(=O)N[C@H](C)c2ccc(Br)c(F)c2)c2c1[C@@H](C)CN(c1ccc(C(=O)N(C)C)cn1)C2] | (S)-N-((R)-1-(4-bromo- 3-fluorophenyl)ethyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 571.3 |
| E159A | Example 127^{b} | 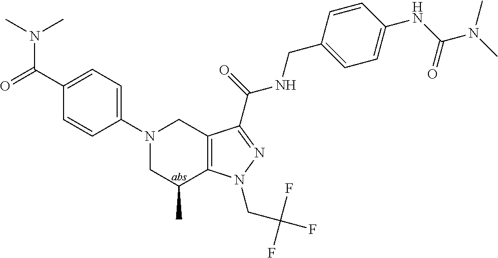 [SMILES: C[C@H]1CN(c2ccc(C(=O)N(C)C)cc2)Cc2c(C(=O)NCc3ccc(NC(=O)N(C)C)cc3)nn(CC(F)(F)F)c21] | (S)-5-(4- (dimethylcarbamoyl) phenyl)-N-(4-(3,3- dimethylureido)benzyl)- 7-methyl-1-(2,2,2- trifluoroethyl)- 4,5,6,7-tetrahydro-1H- pyrazolo[4,3-c]pyridine- 3-carboxamide | 586.3 |
| | | ¹H NMR (400 MHz, (CD₃)₂SO) δ 8.50 (t, | | |
| | | 1H), 8.24 (s, 1H), 7.43-7.32 (m, 4H), | | |
| | | 7.18 (d, 2H), 7.00 (d, 2H), 5.23-5.06 | | |
| | | (m, 2H), 4.57 (d, 1H), 4.36 (d, 2H), 4.16 | | |
| | | (d, 1H), 3.87 (d, 1H), 3.34-3.22 (m, | | |
| | | 2H), 2.98 (s, 6H), 2.92 (s, 6H), 1.31 (d, | | |
| | | 3H). | | |
| E160A | Example 567 | 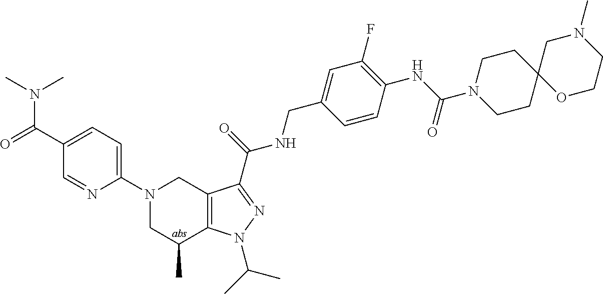 [SMILES: CC(C)n1nc(C(=O)NCc2ccc(NC(=O)N3CCC4(CC3)CN(C)CCO4)c(F)c2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl)- 2-fluorophenyl)-4- methyl-1-oxa-4,9- diazaspiro[5.5]undecane- 9-carboxamide | 690.5 |
| E161A | Example 567 | 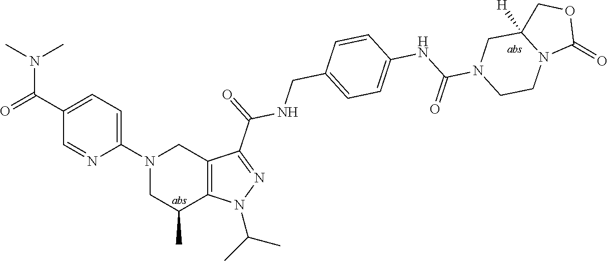 [SMILES: CC(C)n1nc(C(=O)NCc2ccc(NC(=O)N3CCN4C(=O)OC[C@@H]4C3)cc2)c2c1[C@@H](C)CN(c1ccc(C(=O)N(C)C)cn1)C2] | (S)-N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-3-oxotetrahydro- 3H-oxazolo[3,4-a] pyrazine-7(1H)- carboxamide | 644.5 |
| E162A | Example 567 | 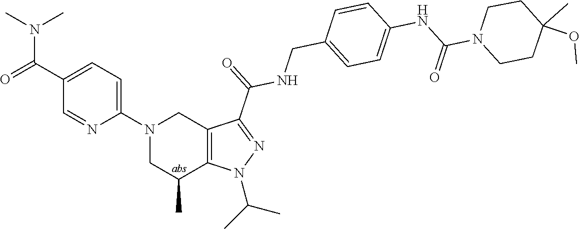 [SMILES: COC1(C)CCN(C(=O)Nc2ccc(CNC(=O)c3nn(C(C)C)c4c3CN(c3ccc(C(=O)N(C)C)cn3)C[C@@H]4C)cc2)CC1] | (S)-5-(5- (dimethylcarbamoyl) pyridin-2-yl)-1-isopropyl-N- (4-(4-methoxy-4- methylpiperidine-1- carboxamido)benzyl)-7- methyl-4,5,6,7-tetrahydro-1H- pyrazolo[4,3-c]pyridine- 3-carboxamide | 631.5 |
| E163A | Example 567 | 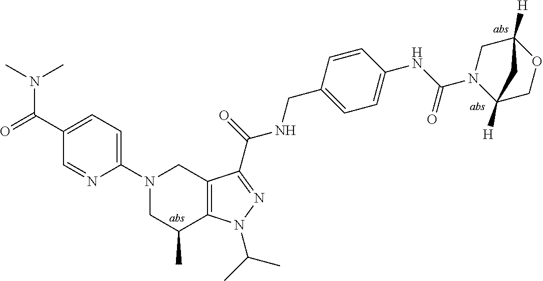 [SMILES: CC(C)n1nc(C(=O)NCc2ccc(NC(=O)N3C[C@H]4C[C@@H]3CO4)cc2)c2c1[C@@H](C)CN(c1ccc(C(=O)N(C)C)cn1)C2] | (1R,4R)-N-(4-(((S)-5- (5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2-oxa-5- azabicyclo[2.2.1] heptane-5-carboxamide | 601.4 |
| E164A | Example 567 | 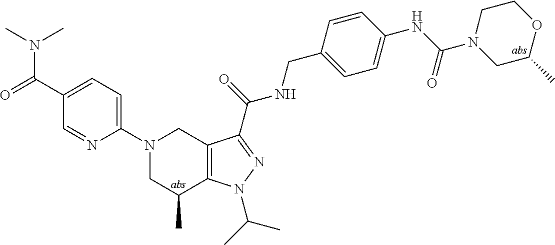 [SMILES: CC(C)n1nc(C(=O)NCc2ccc(NC(=O)N3CCO[C@H](C)C3)cc2)c2c1[C@@H](C)CN(c1ccc(C(=O)N(C)C)cn1)C2] | (R)-N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2- methylmorpholine-4- carboxamide | 603.5 |
| E165A | Example 567 | 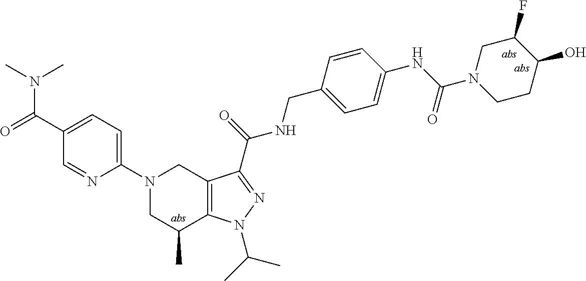 [SMILES: CC(C)n1nc(C(=O)NCc2ccc(NC(=O)N3CC[C@H](O)[C@H](F)C3)cc2)c2c1[C@@H](C)CN(c1ccc(C(=O)N(C)C)cn1)C2] | (S)-5-(5- (dimethylcarbamoyl) pyridin-2-yl)-N-(4- ((3R,4S)-3-fluoro-4- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E166A | Example 567 | 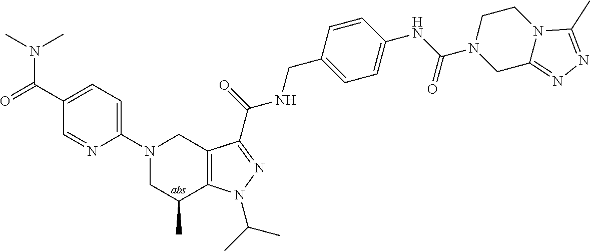 [SMILES: Cc1nnc2n1CCN(C(=O)Nc1ccc(CNC(=O)c3nn(C(C)C)c4c3CN(c3ccc(C(=O)N(C)C)cn3)C[C@@H]4C)cc1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-3-methyl-5,6- dihydro[1,2,4]triazolo [4,3-a]pyrazine-7(8H)- carboxamide | 640.5 |
| E167A | Example 567 | 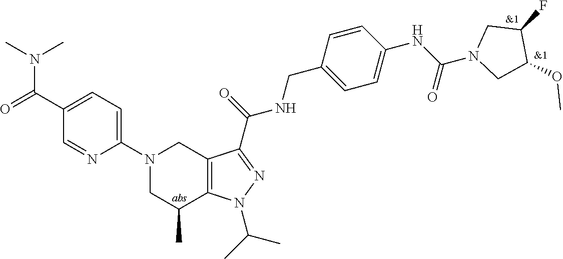 [SMILES: CO[C@@H]1CN(C(=O)Nc2ccc(CNC(=O)c3nn(C(C)C)c4c3CN(c3ccc(C(=O)N(C)C)cn3)C[C@@H]4C)cc2)C[C@H]1F] | (S)-5-(5- (dimethylcarbamoyl) pyridin-2-yl)-N-(4- ((3RS,4RS)-3-fluoro-4- methoxypyrrolidine-1- carboxamido)benzyl)-1- isopropyl-7-methyl- 4,5,6,7-tetrahydro-1H- pyrazolo[4,3-c]pyridine- 3-carboxamide | 621.4 |
| E168A | Example 567 | 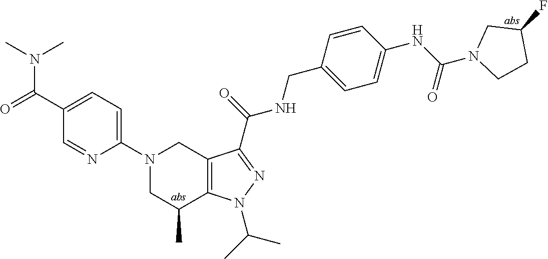 [SMILES: CC(C)n1nc(C(=O)NCc2ccc(NC(=O)N3CC[C@H](F)C3)cc2)c2c1[C@@H](C)CN(c1ccc(C(=O)N(C)C)cn1)C2] | (S)-5-(5- (dimethylcarbamoyl) pyridin-2-yl)-N-(4-((S)-3- fluoropyrrolidine-1- carboxamido)benzyl)-1- isopropyl-7-methyl- 4,5,6,7-tetrahydro-1H- pyrazolo[4,3-c]pyridine- 3-carboxamide | 591.4 |
| E169A | Example 567 | 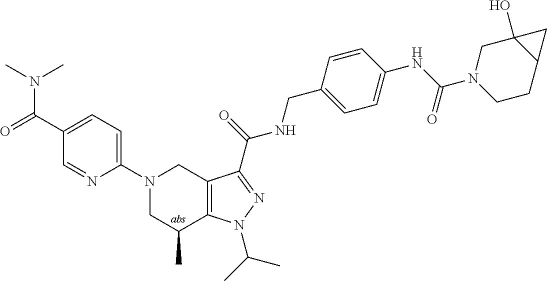 [SMILES: CC(C)n1nc(C(=O)NCc2ccc(NC(=O)N3CCC4CC4(O)C3)cc2)c2c1[C@@H](C)CN(c1ccc(C(=O)N(C)C)cn1)C2] | (7S)-5-(5- (dimethylcarbamoyl) pyridin-2-yl)-N-(4-(1- hydroxy-3- azabicyclo[4.1.0]heptane-3- carboxamido)benzyl)-1- isopropyl-7-methyl- 4,5,6,7-tetrahydro-1H- pyrazolo[4,3-c]pyridine- 3-carboxamide | 615.5 |
| E170A | Example 567 | 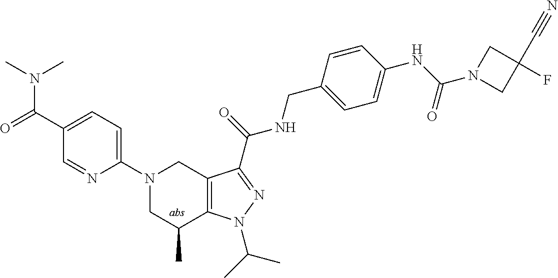 [SMILES: CC(C)n1nc(C(=O)NCc2ccc(NC(=O)N3CC(F)(C#N)C3)cc2)c2c1[C@@H](C)CN(c1ccc(C(=O)N(C)C)cn1)C2] | (S)-N-(4-(3-cyano-3- fluoroazetidine-1- carboxamido)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 602.4 |
| E171A | Example 567 | 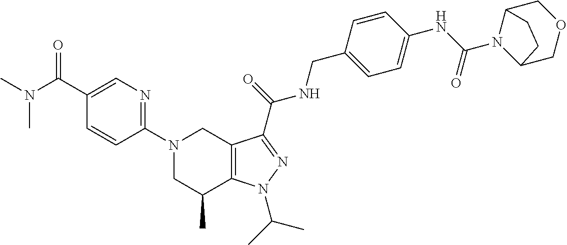 [SMILES: CC(C)n1nc(C(=O)NCc2ccc(NC(=O)N3C4CCC3COC4)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-3-oxa-8- azabicyclo[3.2.1]octane- 8-carboxamide | 615.5 |
| E172A | Example 567 | 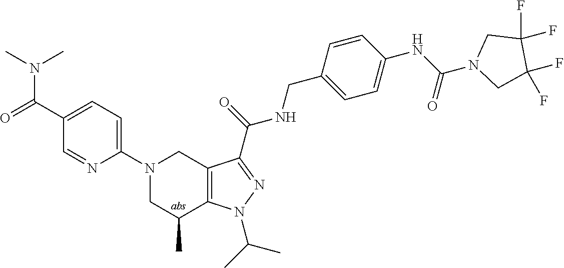 [SMILES: CC(C)n1nc(C(=O)NCc2ccc(NC(=O)N3CC(F)(F)C(F)(F)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(3,3,4,4- tetrafluoropyrrolidine-1- carboxamido)benzyl)- 4,5,6,7-tetrahydro-1H- pyrazolo[4,3-c]pyridine- 3-carboxamide | 645.4 |
| E173A | Example 567 | 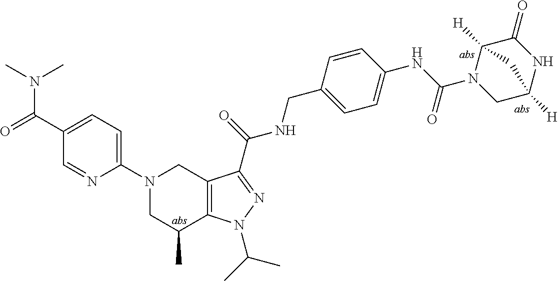 [SMILES: CC(C)n1nc(C(=O)NCc2ccc(NC(=O)N3C[C@H]4C[C@@H]3C(=O)N4)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-((1R,4R)-6- oxo-2,5-diazabicyclo[2.2.1] heptane-2-carboxamido) benzyl)-4,5,6,7-tetrahydro-1H- pyrazolo[4,3-c]pyridine- 3-carboxamide | 614.4 |
| E174A | Example 567 | 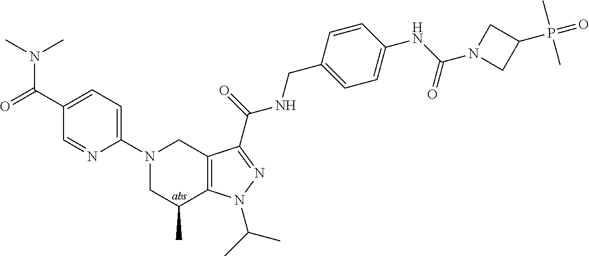 [SMILES: CC(C)n1nc(C(=O)NCc2ccc(NC(=O)N3CC(P(C)(C)=O)C3)cc2)c2c1[C@@H](C)CN(c1ccc(C(=O)N(C)C)cn1)C2] | (S)-5-(5- (dimethylcarbamoyl) pyridin-2-yl)-N-(4-(3- (dimethylphosphoryl) azetidine-1- carboxamido)benzyl)-1- isopropyl-7-methyl- 4,5,6,7-tetrahydro-1H- pyrazolo[4,3-c]pyridine- 3-carboxamide | 635.4 |
| E175A | Example 567 | 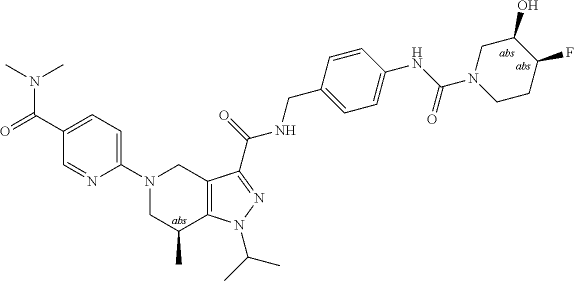 [SMILES: CC(C)n1nc(C(=O)NCc2ccc(NC(=O)N3CC[C@H](F)[C@H](O)C3)cc2)c2c1[C@@H](C)CN(c1ccc(C(=O)N(C)C)cn1)C2] | (S)-5-(5- (dimethylcarbamoyl) pyridin-2-yl)-N-(4- ((3R,4S)-4-fluoro-3- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E176A | Example 567 | 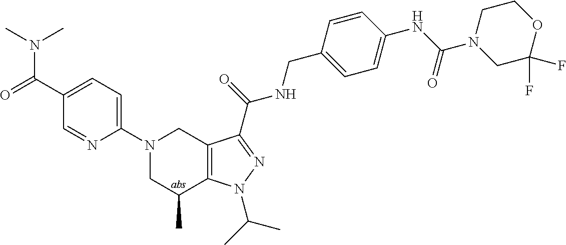 [SMILES: CC(C)n1nc(C(=O)NCc2ccc(NC(=O)N3CCOC(F)(F)C3)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2,2- difluoromorpholine-4- carboxamide | 625.4 |
| E177A | Example 567 | 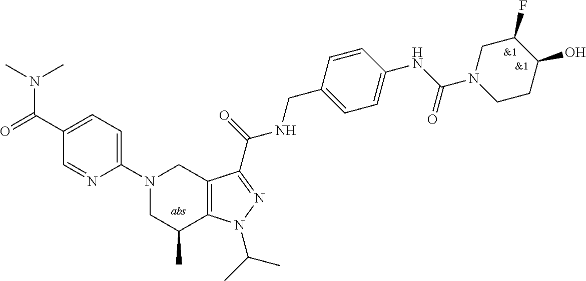 [SMILES: CC(C)n1nc(C(=O)NCc2ccc(NC(=O)N3CC[C@H](O)[C@H](F)C3)cc2)c2c1[C@@H](C)CN(c1ccc(C(=O)N(C)C)cn1)C2] | (S)-5-(5- (dimethylcarbamoyl) pyridin-2-yl)-N-(4- ((3RS,4SR)-3-fluoro-4- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E178A | Example 567 | 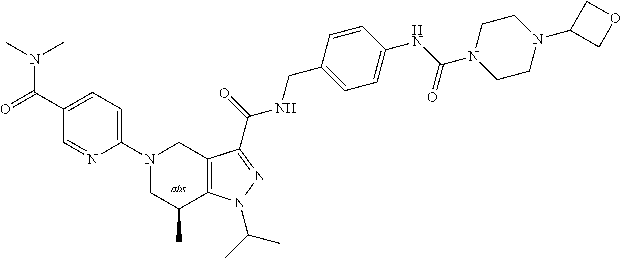 [SMILES: CC(C)n1nc(C(=O)NCc2ccc(NC(=O)N3CCN(C4COC4)CC3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(4-(oxetan- 3-yl)piperazine-1- carboxamido)benzyl)- 4,5,6,7-tetrahydro-1H- pyrazolo[4,3-c]pyridine- 3-carboxamide | 644.5 |
| E179A | Example 567 | 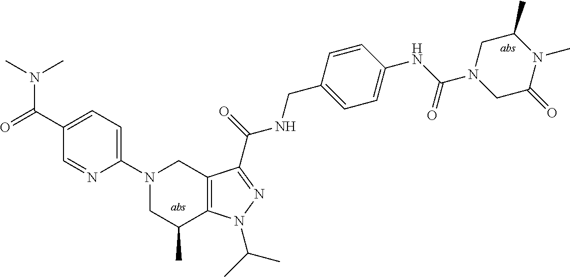 [SMILES: CC(C)n1nc(C(=O)NCc2ccc(NC(=O)N3CC(=O)N(C)[C@H](C)C3)cc2)c2c1[C@@H](C)CN(c1ccc(C(=O)N(C)C)cn1)C2] | (S)-N-(4-((R)-3,4- dimethyl-5- oxopiperazine-1- carboxamido)benzyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 630.5 |
| E180A | Example 567 | 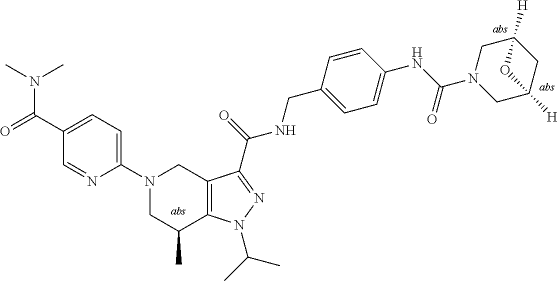 [SMILES: CC(C)n1nc(C(=O)NCc2ccc(NC(=O)N3C[C@H]4C[C@@H](C3)O4)cc2)c2c1[C@@H](C)CN(c1ccc(C(=O)N(C)C)cn1)C2] | (1R,5S)-N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-6-oxa-3- azabicyclo[3.1.1]heptane-3- carboxamide | 601.4 |
| E181A | Example 567 | 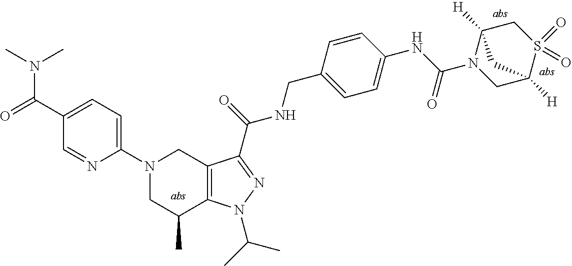 [SMILES: CC(C)n1nc(C(=O)NCc2ccc(NC(=O)N3C[C@H]4C[C@@H]3CS4(=O)=O)cc2)c2c1[C@@H](C)CN(c1ccc(C(=O)N(C)C)cn1)C2] | (1R,4R)-N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2-thia-5- azabicyclo[2.2.1]heptane- 5-carboxamide 2,2- dioxide | 649.4 |
| E182A | Example 567 | 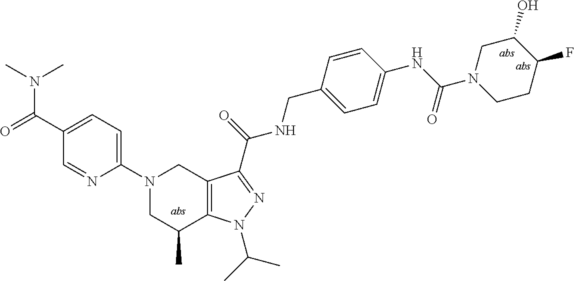 [SMILES: CC(C)n1nc(C(=O)NCc2ccc(NC(=O)N3CC[C@H](F)[C@@H](O)C3)cc2)c2c1[C@@H](C)CN(c1ccc(C(=O)N(C)C)cn1)C2] | (S)-5-(5- (dimethylcarbamoyl) pyridin-2-yl)-N-(4-((3S,4S)- 4-fluoro-3- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E183A | Example 567 | 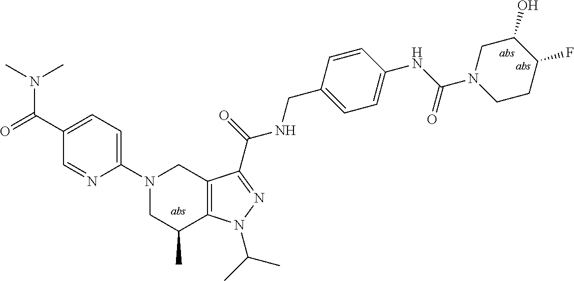 [SMILES: CC(C)n1nc(C(=O)NCc2ccc(NC(=O)N3CC[C@@H](F)[C@@H](O)C3)cc2)c2c1[C@@H](C)CN(c1ccc(C(=O)N(C)C)cn1)C2] | (S)-5-(5- (dimethylcarbamoyl) pyridin-2-yl)-N-(4- ((3S,4R)-4-fluoro-3- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E184A | Example 567 | 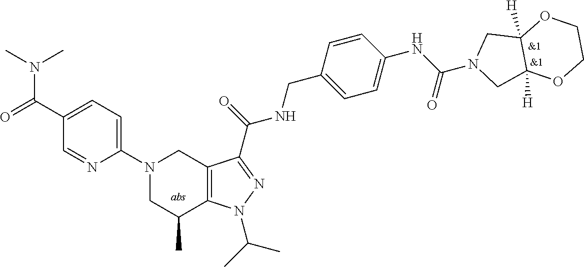 [SMILES: CC(C)n1nc(C(=O)NCc2ccc(NC(=O)N3C[C@@H]4OCCO[C@@H]4C3)cc2)c2c1[C@@H](C)CN(c1ccc(C(=O)N(C)C)cn1)C2] | (4aRS,7aSR)-N-(4- (((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)hexahydro-6H- [1,4]dioxino[2,3-d] pyrrole-6-carboxamide | 631.4 |
| E185A | Example 567 | 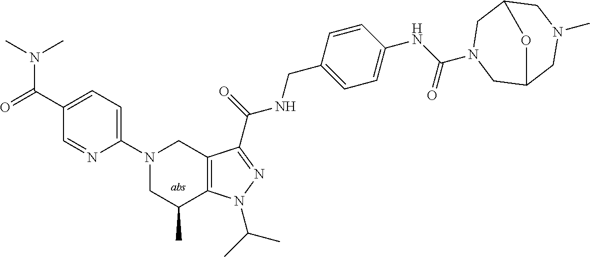 [SMILES: CC(C)n1nc(C(=O)NCc2ccc(NC(=O)N3CC4CN(C)CC(C3)O4)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-7-methyl-9-oxa-3,7- diazabicyclo[3.3.1]nonane- 3-carboxamide | 644.5 |
| E186A | Example 567 | 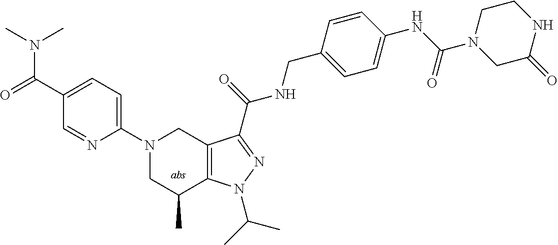 [SMILES: CC(C)n1nc(C(=O)NCc2ccc(NC(=O)N3CCNC(=O)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(3- oxopiperazine-1- carboxamido)benzyl)- 4,5,6,7-tetrahydro-1H- pyrazolo[4,3-c]pyridine- 3-carboxamide | 602.4 |
| E187A | Example 567 | 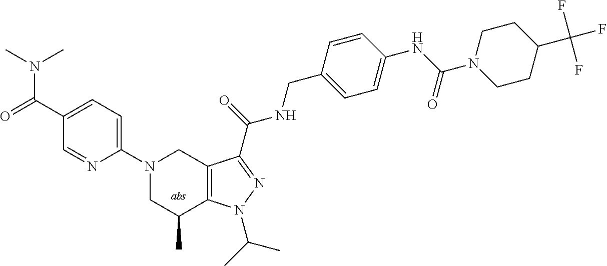 [SMILES: CC(C)n1nc(C(=O)NCc2ccc(NC(=O)N3CCC(C(F)(F)F)CC3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(4- (trifluoromethyl) piperidine-1- carboxamido)benzyl)- 4,5,6,7-tetrahydro-1H- pyrazolo[4,3-c]pyridine- 3-carboxamide | 655.4 |
| E188A | Example 567 | 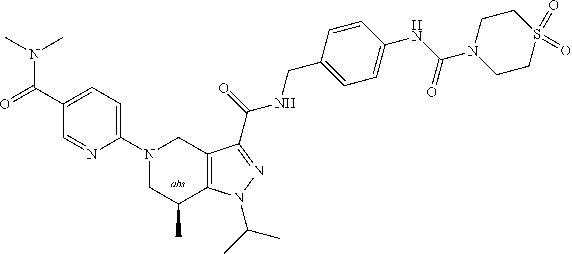 [SMILES: CC(C)n1nc(C(=O)NCc2ccc(NC(=O)N3CCS(=O)(=O)CC3)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)thiomorpholine-4- carboxamide 1,1- dioxide | 637.4 |
| E189A | Example 567 | 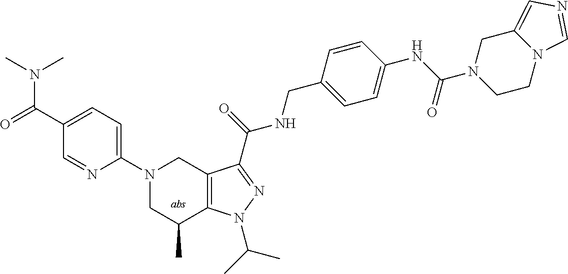 [SMILES: CC(C)n1nc(C(=O)NCc2ccc(NC(=O)N3CCn4cncc4C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(5,6,7,8- tetrahydroimidazo[1,5- a]pyrazine-7- carboxamido)benzyl)- 4,5,6,7-tetrahydro-1H- pyrazolo[4,3-c]pyridine- 3-carboxamide | 625.4 |
| E190A | Example 567 | 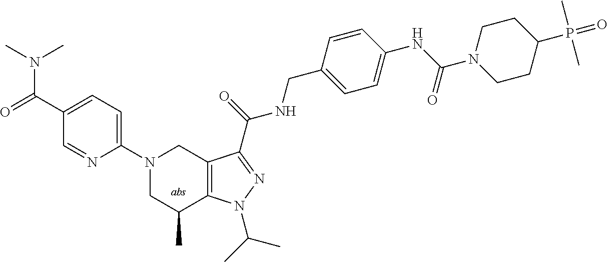 [SMILES: CC(C)n1nc(C(=O)NCc2ccc(NC(=O)N3CCC(P(C)(C)=O)CC3)cc2)c2c1[C@@H](C)CN(c1ccc(C(=O)N(C)C)cn1)C2] | (S)-5-(5- (dimethylcarbamoyl) pyridin-2-yl)-N-(4-(4- (dimethylphosphoryl) piperidine-1- carboxamido)benzyl)-1- isopropyl-7-methyl- 4,5,6,7-tetrahydro-1H- pyrazolo[4,3-c]pyridine- 3-carboxamide | 663.4 |
| E191A | Example 567 | 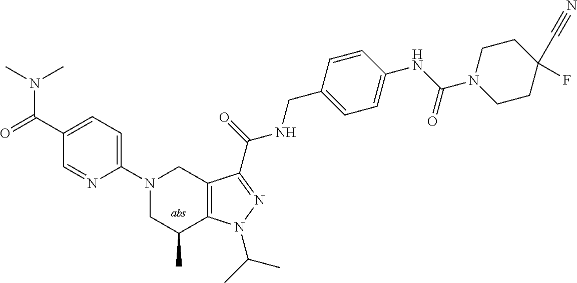 [SMILES: CC(C)n1nc(C(=O)NCc2ccc(NC(=O)N3CCC(F)(C#N)CC3)cc2)c2c1[C@@H](C)CN(c1ccc(C(=O)N(C)C)cn1)C2] | (S)-N-(4-(4-cyano-4- fluoropiperidine-1- carboxamido)benzyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 630.4 |
| E192A | Example 567 | 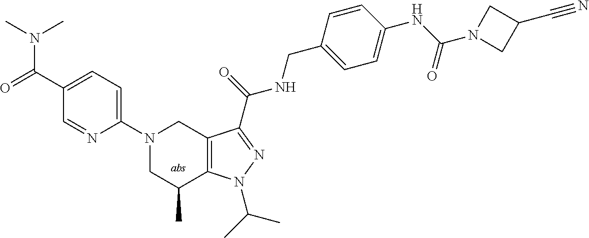 [SMILES: CC(C)n1nc(C(=O)NCc2ccc(NC(=O)N3CC(C#N)C3)cc2)c2c1[C@@H](C)CN(c1ccc(C(=O)N(C)C)cn1)C2] | (S)-N-(4-(3- cyanoazetidine-1- carboxamido)benzyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 584.4 |
| E193A | Example 567 | 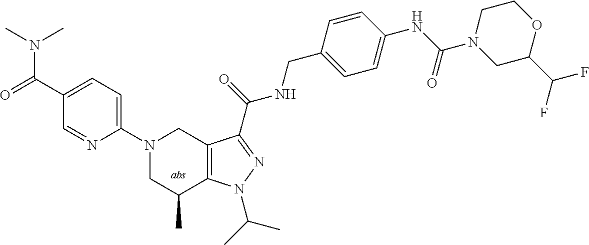 [SMILES: CC(C)n1nc(C(=O)NCc2ccc(NC(=O)N3CCOC(C(F)F)C3)cc2)c2c1[C@@H](C)CN(c1ccc(C(=O)N(C)C)cn1)C2] | 2-(difluoromethyl)-N-(4- (((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)morpholine-4- carboxamide | 639.4 |
| E194A | Example 567 | 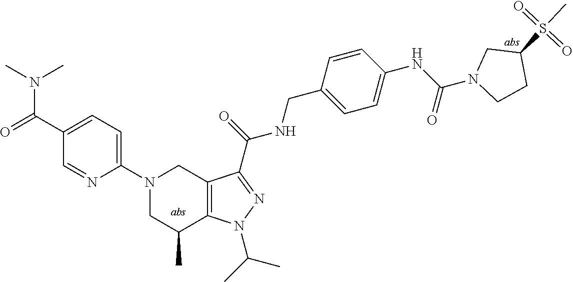 [SMILES: CC(C)n1nc(C(=O)NCc2ccc(NC(=O)N3CC[C@H](S(C)(=O)=O)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-((S)-3- (methylsulfonyl) pyrrolidine-1- carboxamido)benzyl)- 4,5,6,7-tetrahydro-1H- pyrazolo[4,3-c]pyridine- 3-carboxamide | 651.4 |
| E195A | Example 567 | 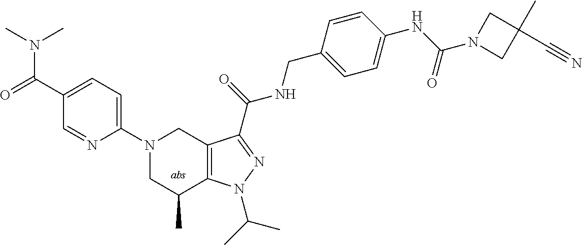 [SMILES: CC(C)n1nc(C(=O)NCc2ccc(NC(=O)N3CC(C)(C#N)C3)cc2)c2c1[C@@H](C)CN(c1ccc(C(=O)N(C)C)cn1)C2] | (S)-N-(4-(3-cyano-3- methylazetidine-1- carboxamido)benzyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 598.5 |
| E196A | Example 567 | 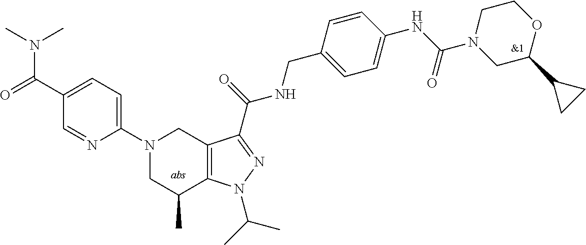 [SMILES: CC(C)n1nc(C(=O)NCc2ccc(NC(=O)N3CCO[C@@H](C4CC4)C3)cc2)c2c1[C@@H](C)CN(c1ccc(C(=O)N(C)C)cn1)C2] | (RS)-2-cyclopropyl-N- (4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)morpholine-4- carboxamide | 629.5 |
| E197A | Example 567 | 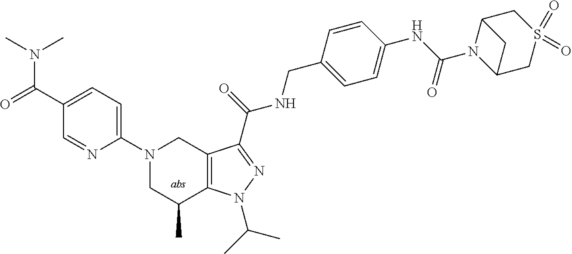 [SMILES: CC(C)n1nc(C(=O)NCc2ccc(NC(=O)N3C4CC3CS(=O)(=O)C4)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-3-thia-6- azabicyclo[3.1.1]heptane-6- carboxamide 3,3-dioxide | 649.4 |
| E198A | Example 567 | 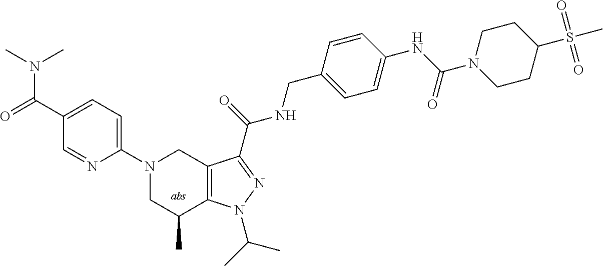 [SMILES: CC(C)n1nc(C(=O)NCc2ccc(NC(=O)N3CCC(S(C)(=O)=O)CC3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(4- (methylsulfonyl)piperidine-1- carboxamido)benzyl)- 4,5,6,7-tetrahydro-1H- pyrazolo[4,3-c]pyridine- 3-carboxamide | 665.4 |
| E199A | Example 567 | 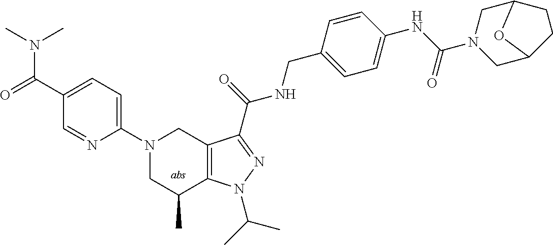 [SMILES: CC(C)n1nc(C(=O)NCc2ccc(NC(=O)N3CC4CCC(C3)O4)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-8-oxa-3- azabicyclo[3.2.1]octane- 3-carboxamide | 615.5 |
| E200A | Example 567 | 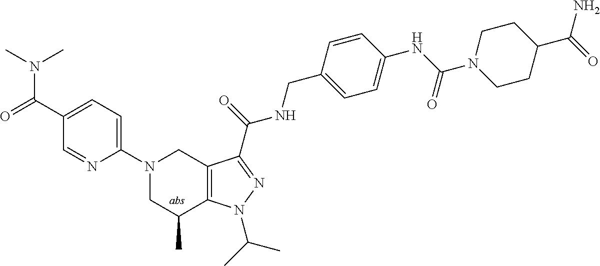 [SMILES: CC(C)n1nc(C(=O)NCc2ccc(NC(=O)N3CCC(C(N)=O)CC3)cc2)c2c1[C@@H](C)CN(c1ccc(C(=O)N(C)C)cn1)C2] | (S)-N1-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)piperidine-1,4- dicarboxamide | 630.5 |
| E201A | Example 567 | 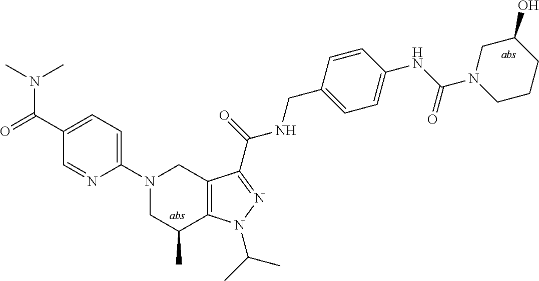 [SMILES: CC(C)n1nc(C(=O)NCc2ccc(NC(=O)N3CCC[C@H](O)C3)cc2)c2c1[C@@H](C)CN(c1ccc(C(=O)N(C)C)cn1)C2] | (S)-5-(5- (dimethylcarbamoyl) pyridin-2-yl)-N-(4-((S)-3- hydroxypiperidine-1- carboxamido)benzyl)-1- isopropyl-7-methyl- 4,5,6,7-tetrahydro-1H- pyrazolo[4,3-c]pyridine- 3-carboxamide | 603.5 |
| E202A | Example 567 | 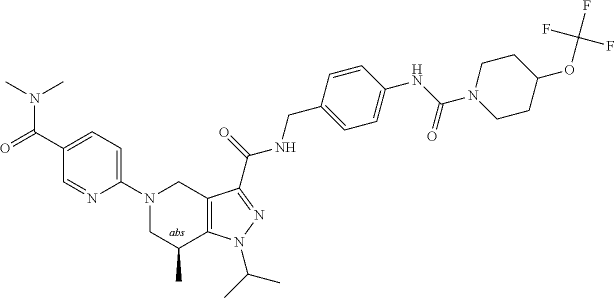 [SMILES: CC(C)n1nc(C(=O)NCc2ccc(NC(=O)N3CCC(OC(F)(F)F)CC3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(4- (trifluoromethoxy) piperidine-1- carboxamido)benzyl)- 4,5,6,7-tetrahydro-1H- pyrazolo[4,3-c]pyridine- 3-carboxamide | 671.4 |
| E203A | Example 567 | 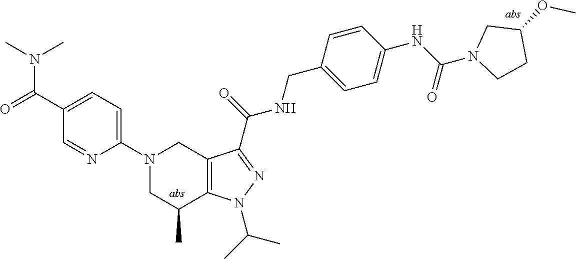 [SMILES: CO[C@@H]1CCN(C(=O)Nc2ccc(CNC(=O)c3nn(C(C)C)c4c3CN(c3ccc(C(=O)N(C)C)cn3)C[C@@H]4C)cc2)C1] | (S)-5-(5- (dimethylcarbamoyl) pyridin-2-yl)-1-isopropyl-N- (4-((R)-3- methoxypyrrolidine-1- carboxamido)benzyl)-7- methyl-4,5,6,7-tetrahydro-1H- pyrazolo[4,3-c]pyridine- 3-carboxamide | 603.5 |
| E204A | Example 567 | 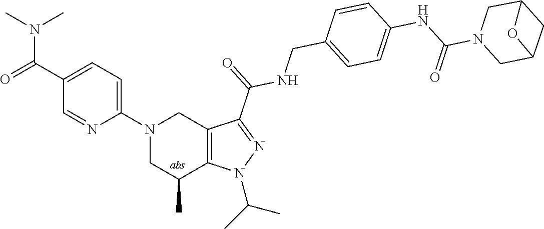 [SMILES: CC(C)n1nc(C(=O)NCc2ccc(NC(=O)N3CC4CC(C3)O4)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-6-oxa-3- azabicyclo[3.1.1]heptane- 3-carboxamide | 601.4 |
| E205A | Example 567 | 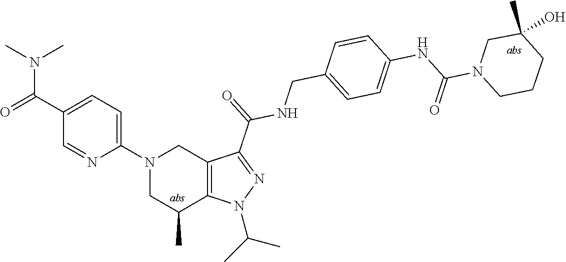 [SMILES: CC(C)n1nc(C(=O)NCc2ccc(NC(=O)N3CCC[C@@](C)(O)C3)cc2)c2c1[C@@H](C)CN(c1ccc(C(=O)N(C)C)cn1)C2] | (S)-5-(5- (dimethylcarbamoyl) pyridin-2-yl)-N-(4-((R)-3- hydroxy-3- methylpiperidine-1- carboxamido)benzyl)-1- isopropyl-7-methyl- 4,5,6,7-tetrahydro-1H- pyrazolo[4,3-c]pyridine- 3-carboxamide | 617.4 |
| E206A | Example 567 | 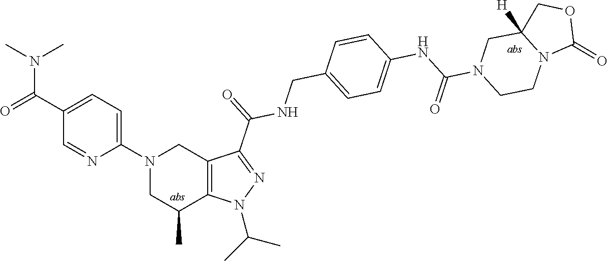 [SMILES: CC(C)n1nc(C(=O)NCc2ccc(NC(=O)N3CCN4C(=O)OC[C@H]4C3)cc2)c2c1[C@@H](C)CN(c1ccc(C(=O)N(C)C)cn1)C2] | (R)-N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-3-oxotetrahydro- 3H-oxazolo[3,4-a] pyrazine-7(1H)-carboxamide | 644.4 |
| E207A | Example 567 | 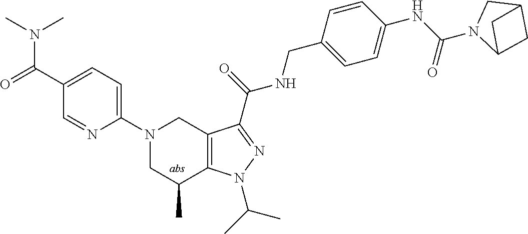 [SMILES: CC(C)n1nc(C(=O)NCc2ccc(NC(=O)N3CC4CC3C4)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2-azabicyclo[2.1.1] hexane-2-carboxamide | 585.4 |
| E208A | Example 567 | 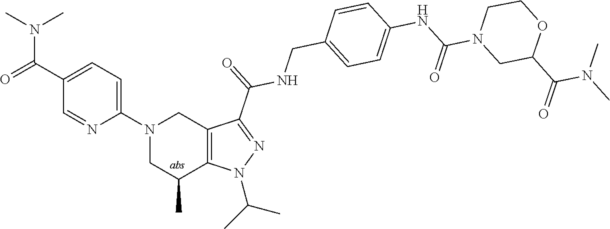 [SMILES: CC(C)n1nc(C(=O)NCc2ccc(NC(=O)N3CCOC(C(=O)N(C)C)C3)cc2)c2c1[C@@H](C)CN(c1ccc(C(=O)N(C)C)cn1)C2] | N4-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-N2,N2- dimethylmorpholine- 2,4-dicarboxamide | 660.5 |
| E209A | Example 567 | 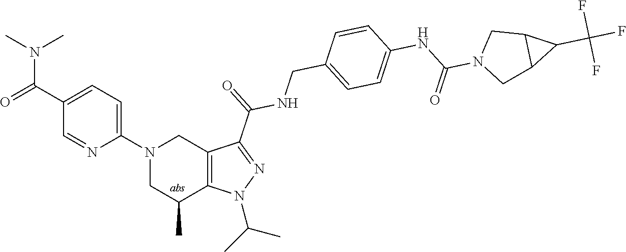 [SMILES: CC(C)n1nc(C(=O)NCc2ccc(NC(=O)N3CC4C(C3)C4C(F)(F)F)cc2)c2c1[C@@H](C)CN(c1ccc(C(=O)N(C)C)cn1)C2] | (7S)-5-(5- (dimethylcarbamoyl) pyridin-2-yl)-1-isopropyl-7- methyl-N-(4-(6- (trifluoromethyl)-3- azabicyclo[3.1.0]hexane-3- carboxamido)benzyl)- 4,5,6,7-tetrahydro-1H- pyrazolo[4,3-c]pyridine- 3-carboxamide | 653.4 |
| E210A | Example 567 | 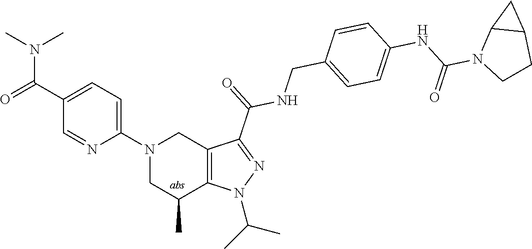 [SMILES: CC(C)n1nc(C(=O)NCc2ccc(NC(=O)N3CCC4CC43)cc2)c2c1[C@@H](C)CN(c1ccc(C(=O)N(C)C)cn1)C2] | (7S)-N-(4-(2- azabicyclo[3.1.0]hexane-2- carboxamido)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 585.4 |
| E211A | Example 567 | 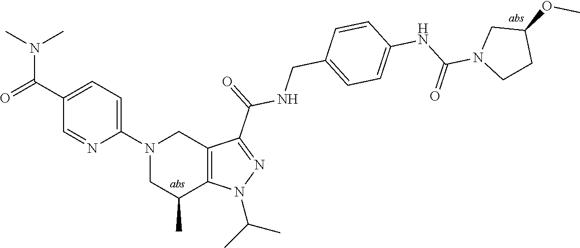 [SMILES: CO[C@H]1CCN(C(=O)Nc2ccc(CNC(=O)c3nn(C(C)C)c4c3CN(c3ccc(C(=O)N(C)C)cn3)C[C@@H]4C)cc2)C1] | (S)-5-(5- (dimethylcarbamoyl) pyridin-2-yl)-1-isopropyl-N- (4-((S)-3- methoxypyrrolidine-1- carboxamido)benzyl)-7- methyl-4,5,6,7-tetrahydro-1H- pyrazolo[4,3-c]pyridine- 3-carboxamide | 603.4 |
| E212A | Example 567 | 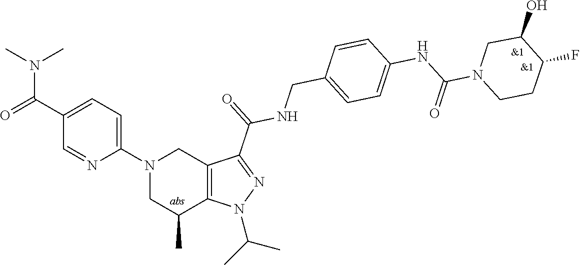 [SMILES: CC(C)n1nc(C(=O)NCc2ccc(NC(=O)N3CC[C@@H](F)[C@H](O)C3)cc2)c2c1[C@@H](C)CN(c1ccc(C(=O)N(C)C)cn1)C2] | (S)-5-(5- (dimethylcarbamoyl) pyridin-2-yl)-N-(4- ((3RS,4RS)-4-fluoro-3- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E213A | Example 567 | 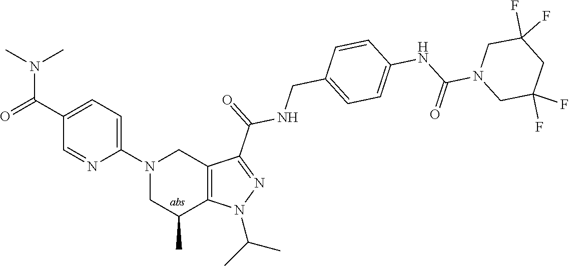 [SMILES: CC(C)n1nc(C(=O)NCc2ccc(NC(=O)N3CC(F)(F)CC(F)(F)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(3,3,5,5- tetrafluoropiperidine-1- carboxamido)benzyl)- 4,5,6,7-tetrahydro-1H- pyrazolo[4,3-c]pyridine- 3-carboxamide | 659.4 |
| E214A | Example 567 | 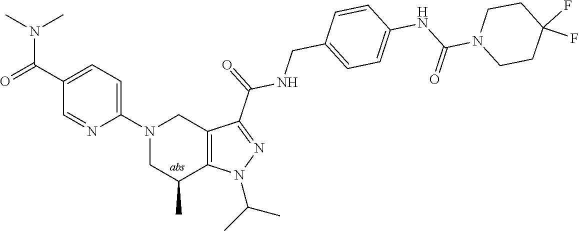 [SMILES: CC(C)n1nc(C(=O)NCc2ccc(NC(=O)N3CCC(F)(F)CC3)cc2)c2c1[C@@H](C)CN(c1ccc(C(=O)N(C)C)cn1)C2] | (S)-N-(4-(4,4- difluoropiperidine-1- carboxamido)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 623.4 |
| E215A | Example 567 | 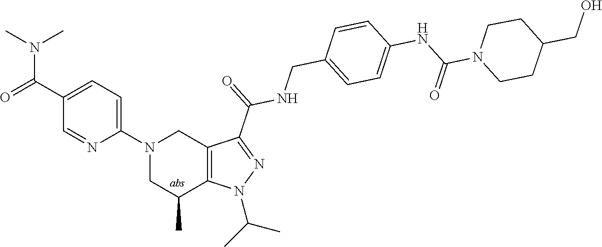 [SMILES: CC(C)n1nc(C(=O)NCc2ccc(NC(=O)N3CCC(CO)CC3)cc2)c2c1[C@@H](C)CN(c1ccc(C(=O)N(C)C)cn1)C2] | (S)-5-(5- (dimethylcarbamoyl) pyridin-2-yl)-N-(4-(4- (hydroxymethyl)piperidine-1- carboxamido)benzyl)-1- isopropyl-7-methyl- 4,5,6,7-tetrahydro-1H- pyrazolo[4,3-c]pyridine- 3-carboxamide | 617.4 |
| E216A | Example 567 | 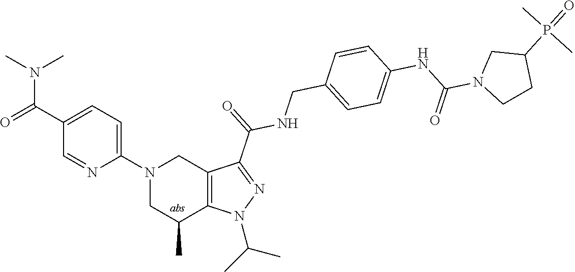 [SMILES: CC(C)n1nc(C(=O)NCc2ccc(NC(=O)N3CCC(P(C)(C)=O)C3)cc2)c2c1[C@@H](C)CN(c1ccc(C(=O)N(C)C)cn1)C2] | (7S)-5-(5- (dimethylcarbamoyl) pyridin-2-yl)-N-(4-(3- (dimethylphosphoryl) pyrrolidine-1- carboxamido)benzyl)-1- isopropyl-7-methyl- 4,5,6,7-tetrahydro-1H- pyrazolo[4,3-c]pyridine- 3-carboxamide | 649.4 |
| E217A | Example 567 | 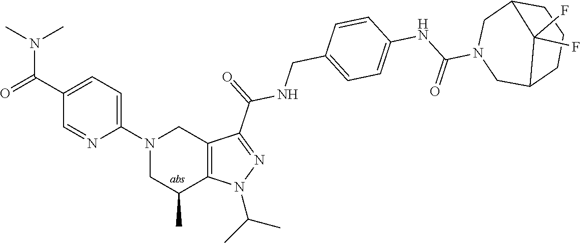 [SMILES: CC(C)n1nc(C(=O)NCc2ccc(NC(=O)N3CC4CCCC(C3)C4(F)F)cc2)c2c1[C@@H](C)CN(c1ccc(C(=O)N(C)C)cn1)C2] | (7S)-N-(4-(9,9-difluoro- 3- azabicyclo[3.3.1]nonane-3- carboxamido)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 663.5 |
| E218A | Example 567 | 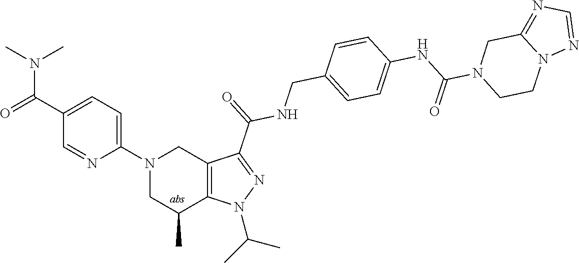 [SMILES: CC(C)n1nc(C(=O)NCc2ccc(NC(=O)N3CCn4ncnc4C3)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-5,6-dihydro- [1,2,4]triazolo[1,5- a]pyrazine-7(8H)- carboxamide | 626.4 |
| E219A | Example 567 | 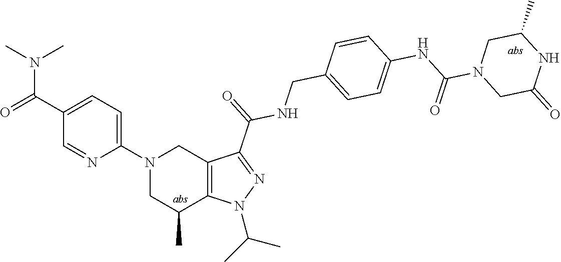 [SMILES: CC(C)n1nc(C(=O)NCc2ccc(NC(=O)N3CC(=O)N[C@@H](C)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-((S)-3-methyl-5- oxopiperazine-1- carboxamido)benzyl)- 4,5,6,7-tetrahydro-1H- pyrazolo[4,3-c]pyridine- 3-carboxamide | 616.4 |
| E220A | Example 567 | 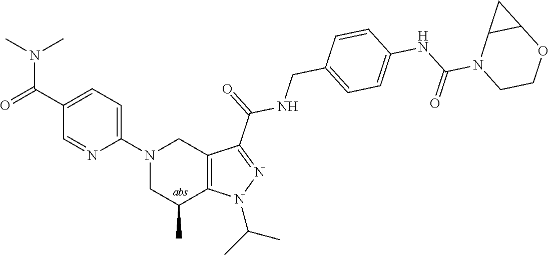 [SMILES: CC(C)n1nc(C(=O)NCc2ccc(NC(=O)N3CCOC4CC43)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2-oxa-5- azabicyclo[4.1.0]heptane- 5-carboxamide | 601.4 |
| E221A | Example 567 | 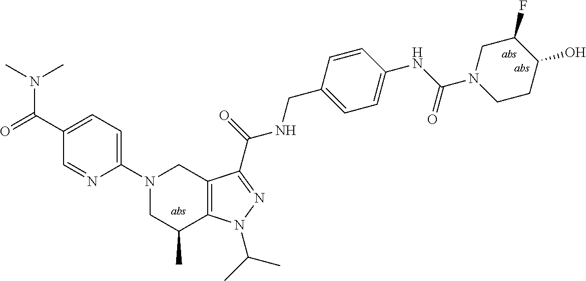 [SMILES: CC(C)n1nc(C(=O)NCc2ccc(NC(=O)N3CC[C@@H](O)[C@H](F)C3)cc2)c2c1[C@@H](C)CN(c1ccc(C(=O)N(C)C)cn1)C2] | (S)-5-(5- (dimethylcarbamoyl) pyridin-2-yl)-i-(4-((3R,4R)- 3-fluoro-4- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E222A | Example 567 | 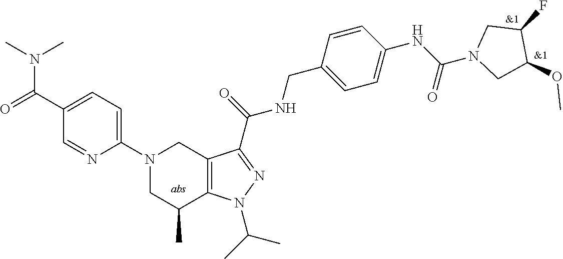 [SMILES: CO[C@H]1CN(C(=O)Nc2ccc(CNC(=O)c3nn(C(C)C)c4c3CN(c3ccc(C(=O)N(C)C)cn3)C[C@@H]4C)cc2)C[C@H]1F] | (S)-5-(5- (dimethylcarbamoyl) pyridin-2-yl)-N-(4- ((3RS,4SR)-3-fluoro-4- methoxypyrrolidine-1- carboxamido)benzyl)-1- isopropyl-7-methyl- 4,5,6,7-tetrahydro-1H- pyrazolo[4,3-c]pyridine- 3-carboxamide | 621.4 |
| E223A | Example 567 | 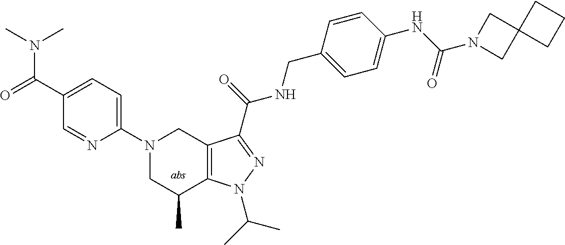 [SMILES: CC(C)n1nc(C(=O)NCc2ccc(NC(=O)N3CC4(CCC4)C3)cc2)c2c1[C@@H](C)CN(c1ccc(C(=O)N(C)C)cn1)C2] | (S)-N-(4-(2- azaspiro[3.3]heptane- 2-carboxamido)benzyl)- 5-(5- (dimethylcarbamoyl) pyridin-2-yl)-1-isopropyl-7- methyl-4,5,6,7-tetrahydro-1H- pyrazolo[4,3-c]pyridine- 3-carboxamide | 599.5 |
| E224A | Example 567 | 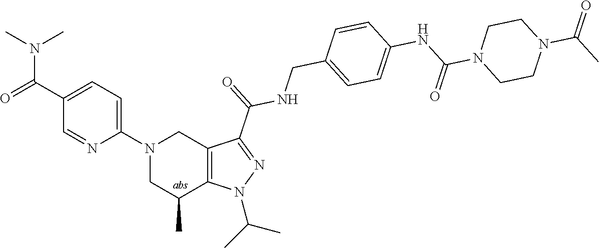 [SMILES: CC(=O)N1CCN(C(=O)Nc2ccc(CNC(=O)c3nn(C(C)C)c4c3CN(c3ccc(C(=O)N(C)C)cn3)C[C@@H]4C)cc2)CC1] | (S)-N-(4-(4- acetylpiperazine-1- carboxamido)benzyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 630.5 |
| E225A | Example 567 | 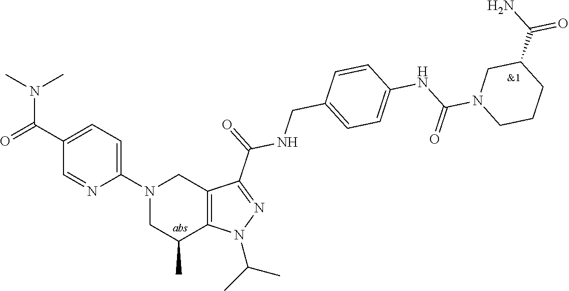 [SMILES: CC(C)n1nc(C(=O)NCc2ccc(NC(=O)N3CCC[C@@H](C(N)=O)C3)cc2)c2c1[C@@H](C)CN(c1ccc(C(=O)N(C)C)cn1)C2] | (RS)-N1-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)piperidine-1,3- dicarboxamide | 630.5 |
| E226A | Example 567 | 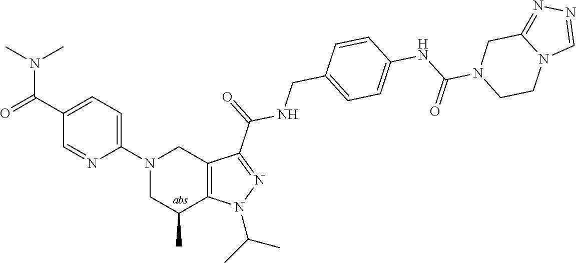 [SMILES: CC(C)n1nc(C(=O)NCc2ccc(NC(=O)N3CCn4cnnc4C3)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-5,6-dihydro- [1,2,4]triazolo[4,3- a]pyrazine-7(8H)- carboxamide | 626.4 |
| E227A | Example 567 | 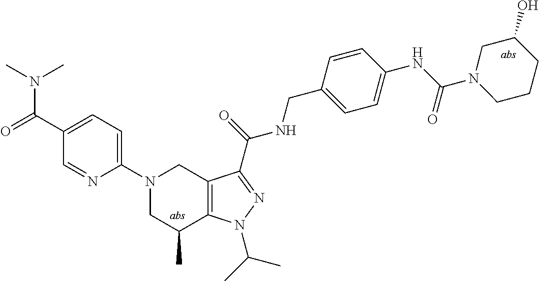 [SMILES: CC(C)n1nc(C(=O)NCc2ccc(NC(=O)N3CCC[C@@H](O)C3)cc2)c2c1[C@@H](C)CN(c1ccc(C(=O)N(C)C)cn1)C2] | (S)-5-(5- (dimethylcarbamoyl) pyridin-2-yl)-N-(4-((R)-3- hydroxypiperidine-1- carboxamido)benzyl)-1- isopropyl-7-methyl- 4,5,6,7-tetrahydro-1H- pyrazolo[4,3-c]pyridine- 3-carboxamide | 603.5 |
| E228A | Example 567 | 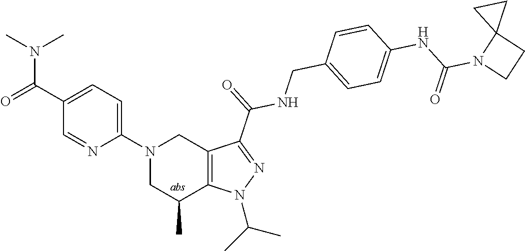 [SMILES: CC(C)n1nc(C(=O)NCc2ccc(NC(=O)N3CCC34CC4)cc2)c2c1[C@@H](C)CN(c1ccc(C(=O)N(C)C)cn1)C2] | (S)-N-(4-(4- azaspiro[2.3]hexane-4- carboxamido)benzyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 585.4 |
| E229A | Example 567 | 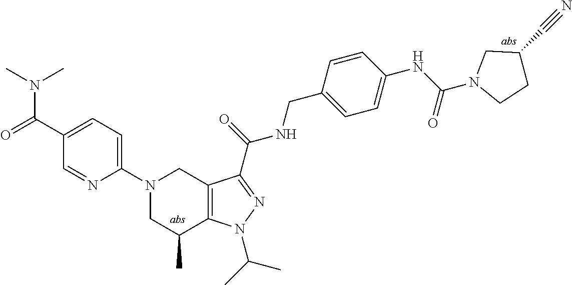 [SMILES: CC(C)n1nc(C(=O)NCc2ccc(NC(=O)N3CC[C@@H](C#N)C3)cc2)c2c1[C@@H](C)CN(c1ccc(C(=O)N(C)C)cn1)C2] | (S)-N-(4-((R)-3- cyanopyrrolidine-1- carboxamido)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 598.4 |
| E230A | Example 567 | 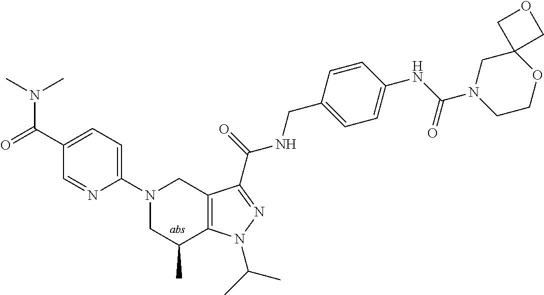 [SMILES: CC(C)n1nc(C(=O)NCc2ccc(NC(=O)N3CCOC4(COC4)C3)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2,5-dioxa-8- azaspiro[3.5]nonane-8- carboxamide | 631.4 |
| E231A | Example 567 | 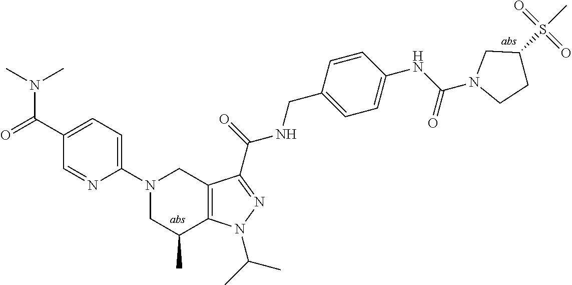 [SMILES: CC(C)n1nc(C(=O)NCc2ccc(NC(=O)N3CC[C@@H](S(C)(=O)=O)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-((R)-3- (methylsulfonyl) pyrrolidine-1- carboxamido)benzyl)- 4,5,6,7-tetrahydro-1H- pyrazolo[4,3-c]pyridine- 3-carboxamide | 651.4 |
| E232A | Example 567 | 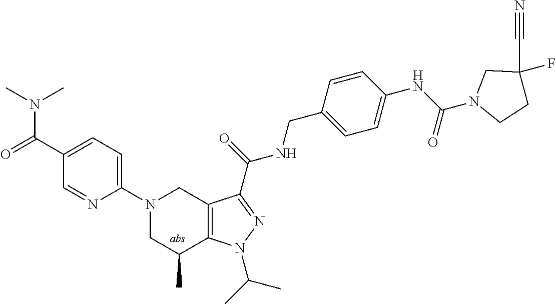 [SMILES: CC(C)n1nc(C(=O)NCc2ccc(NC(=O)N3CCC(F)(C#N)C3)cc2)c2c1[C@@H](C)CN(c1ccc(C(=O)N(C)C)cn1)C2] | (7S)-N-(4-(3-cyano-3- fluoropyrrolidine-1- carboxamido)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 616.4 |
| E233A | Example 567 | 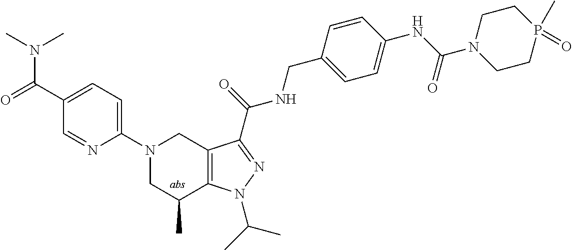 [SMILES: CC(C)n1nc(C(=O)NCc2ccc(NC(=O)N3CCP(C)(=O)CC3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(4-methyl- 4-oxido-1,4- azaphosphinane-1- carboxamido)benzyl)- 4,5,6,7-tetrahydro-1H- pyrazolo[4,3-c]pyridine- 3-carboxamide | 635.5 |
| E234A | Example 567 | 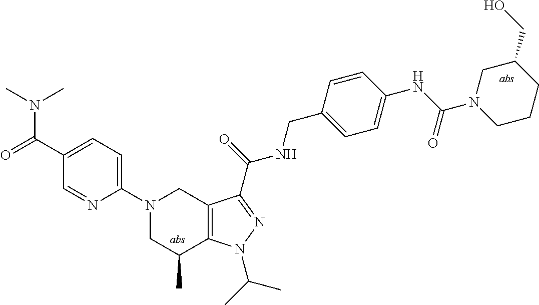 [SMILES: CC(C)n1nc(C(=O)NCc2ccc(NC(=O)N3CCC[C@@H](CO)C3)cc2)c2c1[C@@H](C)CN(c1ccc(C(=O)N(C)C)cn1)C2] | (S)-5-(5- (dimethylcarbamoyl) pyridin-2-yl)-N-(4-((R)-3- (hydroxymethyl) piperidine-1- carboxamido)benzyl)-1- isopropyl-7-methyl- 4,5,6,7-tetrahydro-1H- pyrazolo[4,3-c]pyridine- 3-carboxamide | 617.4 |
| E235A | Example 567 | 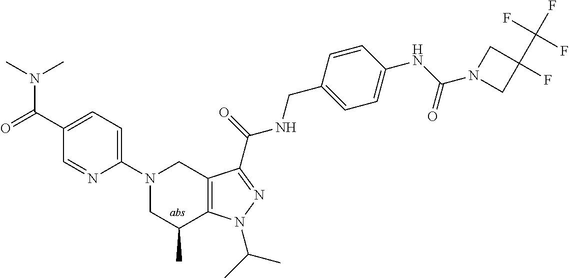 [SMILES: CC(C)n1nc(C(=O)NCc2ccc(NC(=O)N3CC(F)(C(F)(F)F)C3)cc2)c2c1[C@@H](C)CN(c1ccc(C(=O)N(C)C)cn1)C2] | (S)-5-(5- (dimethylcarbamoyl) pyridin-2-yl)-N-(4-(3-fluoro- 3-(trifluoromethyl) azetidine-1- carboxamido)benzyl)-1- isopropyl-7-methyl- 4,5,6,7-tetrahydro-1H- pyrazolo[4,3-c]pyridine- 3-carboxamide | 645.4 |
| E236A | Example 567 | 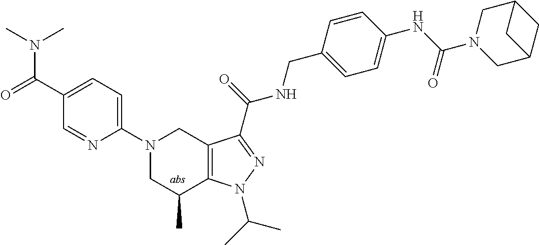 [SMILES: CC(C)n1nc(C(=O)NCc2ccc(NC(=O)N3CC4CC(C4)C3)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-3- azabicyclo[3.1.1]heptane-3- carboxamide | 599.5 |
| E237A | Example 567 | 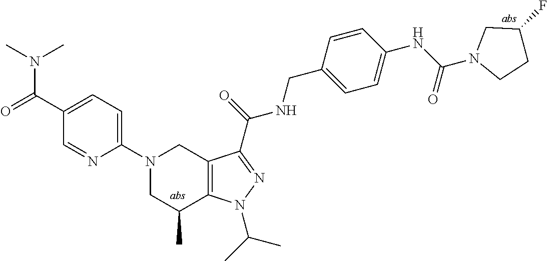 [SMILES: CC(C)n1nc(C(=O)NCc2ccc(NC(=O)N3CC[C@@H](F)C3)cc2)c2c1[C@@H](C)CN(c1ccc(C(=O)N(C)C)cn1)C2] | (S)-5-(5- (dimethylcarbamoyl) pyridin-2-yl)-N-(4-((R)-3- fluoropyrrolidine-1- carboxamido)benzyl)-1- isopropyl-7-methyl- 4,5,6,7-tetrahydro-1H- pyrazolo[4,3-c]pyridine- 3-carboxamide | 591.4 |
| E238A | Example 567 | 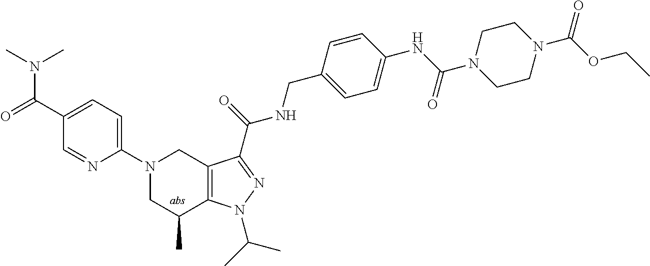 [SMILES: CCOC(=O)N1CCN(C(=O)Nc2ccc(CNC(=O)c3nn(C(C)C)c4c3CN(c3ccc(C(=O)N(C)C)cn3)C[C@@H]4C)cc2)CC1] | ethyl (S)-4-((4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)carbamoyl) piperazine-1-carboxylate | 660.5 |
| E239A | Example 567 | 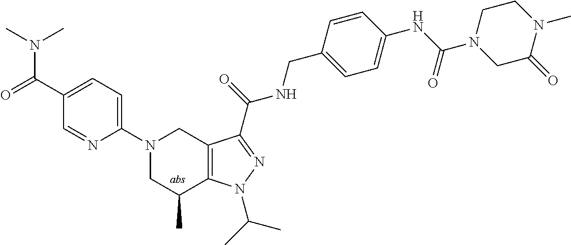 [SMILES: CC(C)n1nc(C(=O)NCc2ccc(NC(=O)N3CCN(C)C(=O)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(4-methyl- 3-oxopiperazine-1- carboxamido)benzyl)- 4,5,6,7-tetrahydro-1H- pyrazolo[4,3-c]pyridine- 3-carboxamide | 616.5 |
| E240A | Example 567 | 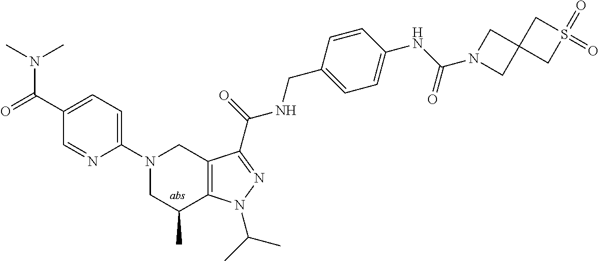 [SMILES: CC(C)n1nc(C(=O)NCc2ccc(NC(=O)N3CC4(C3)CS(=O)(=O)C4)cc2)c2c1[C@@H](C)CN(c1ccc(C(=O)N(C)C)cn1)C2] | (S)-N-(4-((5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2-thia-6- azaspiro[3.3]heptane- 6-carboxamide 2,2- dioxide | 649.4 |
| E241A | Example 567 | 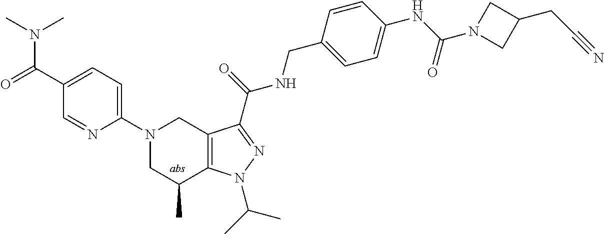 [SMILES: CC(C)n1nc(C(=O)NCc2ccc(NC(=O)N3CC(CC#N)C3)cc2)c2c1[C@@H](C)CN(c1ccc(C(=O)N(C)C)cn1)C2] | (S)-N-(4-(3- (cyanomethyl)azetidine-1- carboxamido)benzyl)-5-(5- (dimethylcarbamoyl) pyridin-2-yl)-1-isopropyl-7- methyl-4,5,6,7-tetrahydro-1H- pyrazolo[4,3-c]pyridine- 3-carboxamide | 598.4 |
| E242A | Example 567 | 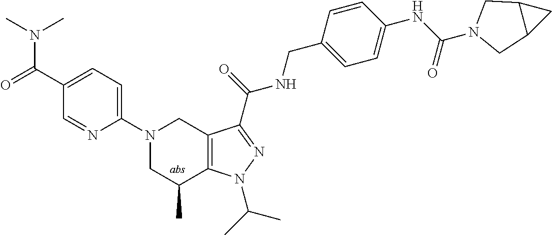 [SMILES: CC(C)n1nc(C(=O)NCc2ccc(NC(=O)N3CC4CC4C3)cc2)c2c1[C@@H](C)CN(c1ccc(C(=O)N(C)C)cn1)C2] | (7S)-N-(4-(3- azabicyclo[3.1.0]hexane-3- carboxamido)benzyl)-5- (5- (dimethylcarbamoyl) pyridin-2-yl)-1-isopropyl-7- methyl-4,5,6,7-tetrahydro-1H- pyrazolo[4,3-c]pyridine- 3-carboxamide | 585.4 |
| E243A | Example 567 | 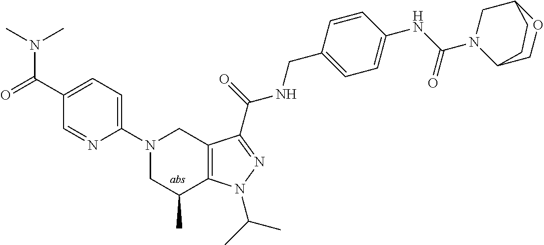 [SMILES: CC(C)n1nc(C(=O)NCc2ccc(NC(=O)N3CC4CCC3CO4)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2-oxa-5- azabicyclo[2.2.2]octane-5- carboxamide | 615.4 |
| E244A | Example 567 | 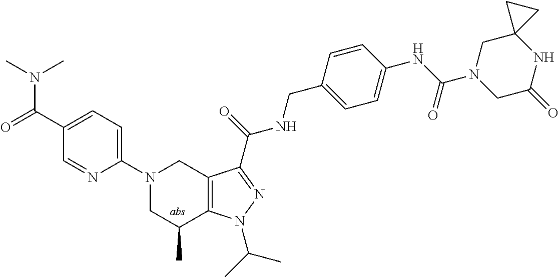 [SMILES: CC(C)n1nc(C(=O)NCc2ccc(NC(=O)N3CC(=O)NC4(CC4)C3)cc2)c2c1[C@@H](C)CN(c1ccc(C(=O)N(C)C)cn1)C2] | (S)-5-(5- (dimethylcarbamoyl) pyridin-2-yl)-1-isopropyl-7- methyl-N-(4-(5-oxo-4,7- diazaspiro[2.5]octane- 7-carboxamido)benzyl)- 4,5,6,7-tetrahydro-1H- pyrazolo[4,3-c]pyridine- 3-carboxamide | 628.5 |
| E245A | Example 567 | 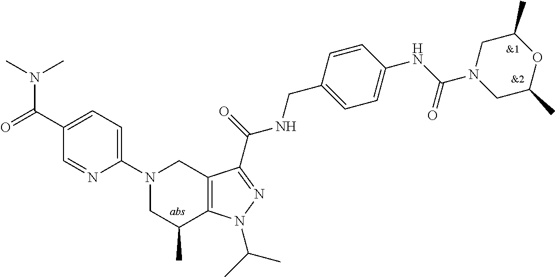 [SMILES: CC(C)n1nc(C(=O)NCc2ccc(NC(=O)N3C[C@@H](C)O[C@@H](C)C3)cc2)c2c1[C@@H](C)CN(c1ccc(C(=O)N(C)C)cn1)C2] | (2RS,6SR)-N-(4-(((S)- 5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2,6- dimethylmorpholine-4- carboxamide | 617.4 |
| E246A | Example 567 | 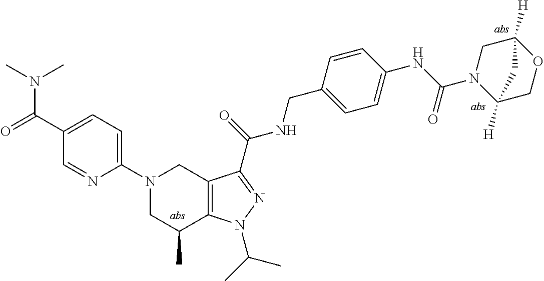 [SMILES: CC(C)n1nc(C(=O)NCc2ccc(NC(=O)N3C[C@@H]4C[C@H]3CO4)cc2)c2c1[C@@H](C)CN(c1ccc(C(=O)N(C)C)cn1)C2] | (1S,4S)-N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-2-oxa-5- azabicyclo[2.2.1]heptane- 5-carboxamide | 601.4 |
| E247A | Example 567 | 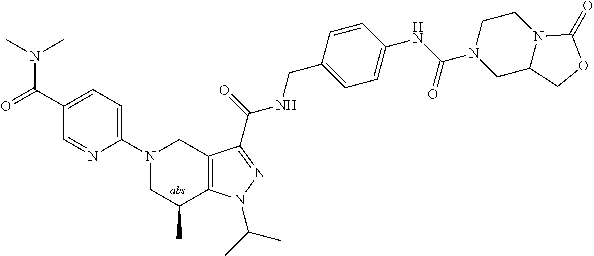 [SMILES: CC(C)n1nc(C(=O)NCc2ccc(NC(=O)N3CCN4C(=O)OCC4C3)cc2)c2c1[C@@H](C)CN(c1ccc(C(=O)N(C)C)cn1)C2] | N-(4-(((S)-5-(5- (dimethylcarbamoyl) pyridin-2-yl)-1-isopropyl-7- methyl-4,5,6,7-tetrahydro-1H- pyrazolo[4,3-c]pyridine- 3-carboxamido)methyl) phenyl)-3-oxotetrahydro- 3H-oxazolo[3,4- a]pyrazine-7(1H)- carboxamide | 644.4 |
| E248A | Example 567 | 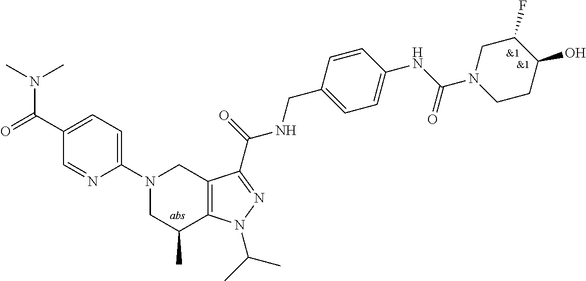 [SMILES: CC(C)n1nc(C(=O)NCc2ccc(NC(=O)N3CC[C@H](O)[C@@H](F)C3)cc2)c2c1[C@@H](C)CN(c1ccc(C(=O)N(C)C)cn1)C2] | (S)-5-(5- (dimethylcarbamoyl) pyridin-2-yl)-N-(4- ((3RS,4RS)-3-fluoro-4- hydroxypiperidine-1- carboxamido)benzyl)-1- isopropyl-7-methyl- 4,5,6,7-tetrahydro-1H- pyrazolo[4,3-c]pyridine- 3-carboxamide | 621.4 |
| E249A | Example 277 | 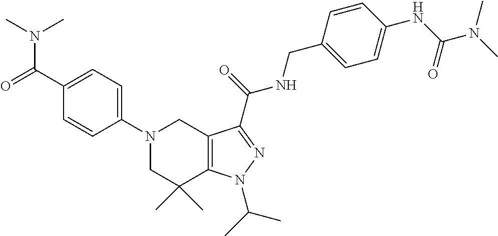 [SMILES: CC(C)n1nc(C(=O)NCc2ccc(NC(=O)N(C)C)cc2)c2c1C(C)(C)CN(c1ccc(C(=O)N(C)C)cc1)C2] | 5-(4- (dimethylcarbamoyl) phenyl)-N-(4-(3,3- dimethylureido)benzyl)- 1-isopropyl-7,7- dimethyl-4,5,6,7- tetrahydro-1H- pyrazolo[4,3-c]pyridine- 3-carboxamide | 560.4 |
| E250A | Example 127^{b} | 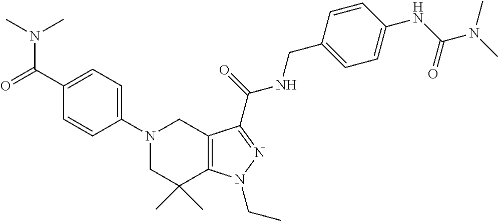 [SMILES: CCn1nc(C(=O)NCc2ccc(NC(=O)N(C)C)cc2)c2c1C(C)(C)CN(c1ccc(C(=O)N(C)C)cc1)C2] | 5-(4- (dimethylcarbamoyl) phenyl)-N-(4-(3,3- dimethylureido)benzyl)- 1-ethyl-7,7-dimethyl- 4,5,6,7-tetrahydro-1H- pyrazolo[4,3-c]pyridine- 3-carboxamide | 546.3 |
| E251A | Example 127^{b} | 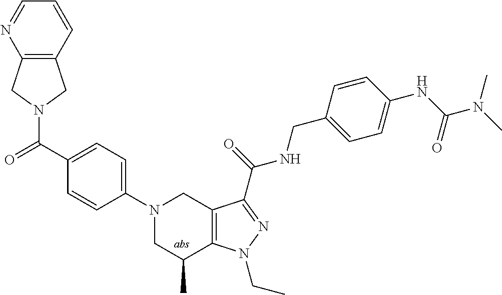 [SMILES: CCn1nc(C(=O)NCc2ccc(NC(=O)N(C)C)cc2)c2c1[C@@H](C)CN(c1ccc(C(=O)N3Cc4cccnc4C3)cc1)C2] | (S)-5-(4-(6,7-dihydro- 5H-pyrrolo[3,4- b]pyridine-6- carbonyl)phenyl)-N-(4- (3,3- dimethylureido)benzyl)- 1-ethyl-7-methyl- 4,5,6,7-tetrahydro-1H- pyrazolo[4,3-c]pyridine- 3-carboxamide | 607.3 |
| E252A | Example 127^{b} | 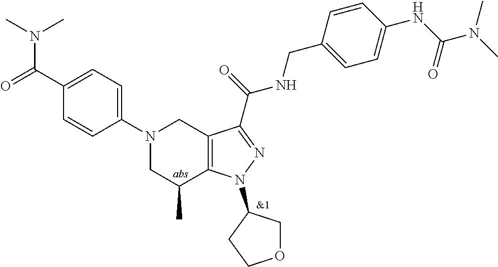 [SMILES: C[C@H]1CN(c2ccc(C(=O)N(C)C)cc2)Cc2c(C(=O)NCc3ccc(NC(=O)N(C)C)cc3)nn([C@@H]3CCOC3)c21] | (S)-5-(4- (dimethylcarbamoyl) phenyl)-N-(4-(3,3- dimethylureido)benzyl)- 7-methyl-1-((RS)- tetrahydrofuran-3-yl)- 4,5,6,7-tetrahydro-1H- pyrazolo[4,3-c]pyridine- 3-carboxamide | 574.3 |
| E253A | Preparation 45/ Example 127 | 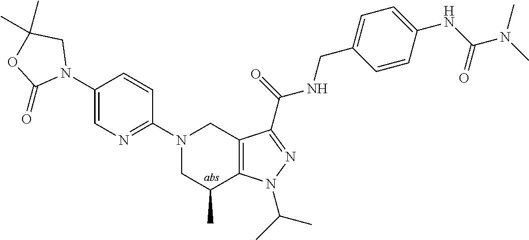 | (S)-5-(5-(5,5-dimethyl- 2-oxooxazolidin-3- yl)pyridin-2-yl)-N-(4-(3,3- dimethylureido)benzyl)- 1-isopropyl-7-methyl- 4,5,6,7-tetrahydro-1H- pyrazolo[4,3-c]pyridine- 3-carboxamide | 589.3 |
| E254A * | Example 127^{c} | 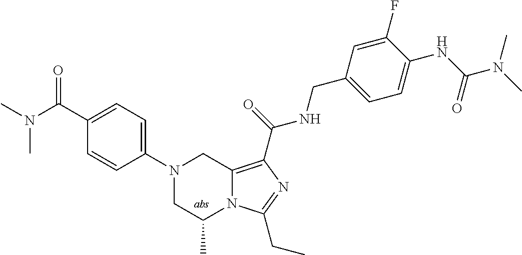 | (R)-7-(4- (dimethylcarbamoyl) phenyl)-N-(4-(3,3- dimethylureido)-3- fluorobenzyl)-3-ethyl-5- methyl-5,6,7,8- tetrahydroimidazo[1,5-a] pyrazine-1-carboxamide | 550.3 |
| E255A * | Example 127^{c} | 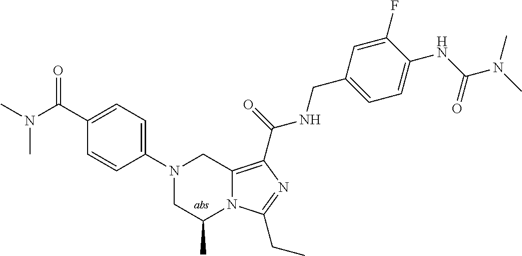 | (S)-7-(4- (dimethylcarbamoyl) phenyl)-N-(4-(3,3- dimethylureido)-3- fluorobenzyl)-3-ethyl-5- methyl-5,6,7,8- tetrahydroimidazo[1,5-a] pyrazine-1-carboxamide | 550.3 |
| E256A * | Example 127^{c} | 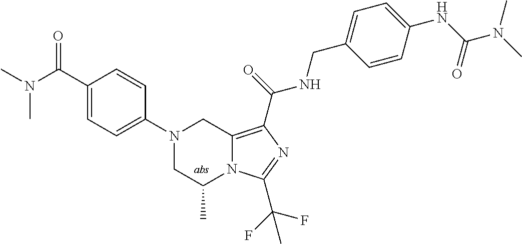 | (R)-3-(1,1- difluoroethyl)-7-(4- (dimethylcarbamoyl) phenyl)-N-(4-(3,3- dimethylureido)benzyl)- 5-methyl-5,6,7,8- tetrahydroimidazo[1,5-a] pyrazine-1-carboxamide | 568.3 |
| E257A * | Example 127^{c} | 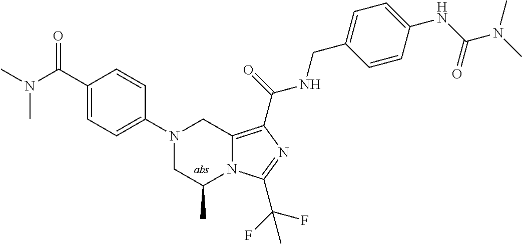 | (S)-3-(1,1- difluoroethyl)-7-(4- (dimethylcarbamoyl) phenyl)-N-(4-(3,3- dimethylureido)benzyl)- 5-methyl-5,6,7,8- tetrahydroimidazo[1,5-a] pyrazine-1-carboxamide | 568.3 |
| E258A * | Example 127^{c} | 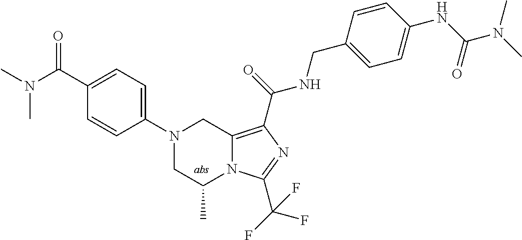 | (R)-7-(4- (dimethylcarbamoyl) phenyl)-N-(4-(3,3- dimethylureido)benzyl)- 5-methyl-3- (trifluoromethyl)- 5,6,7,8- tetrahydroimidazo[1,5-a] pyrazine-1-carboxamide | 572.3 |
| E259A * | Example 127^{c} | 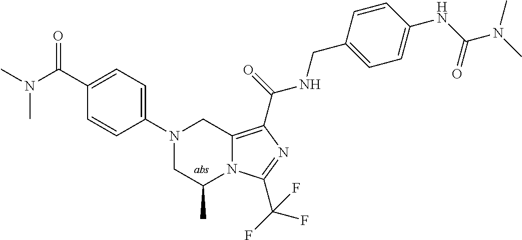 | (S)-7-(4- (dimethylcarbamoyl) phenyl)-N-(4-(3,3- dimethylureido)benzyl)- 5-methyl-3- (trifluoromethyl)- 5,6,7,8- tetrahydroimidazo[1,5-a] pyrazine-1-carboxamide | 572.4 |
| E260A * | Example 127 | 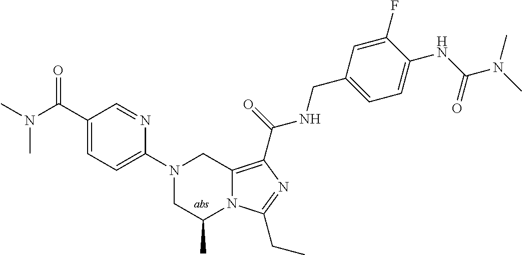 | (S)-7-(5- (dimethylcarbamoyl) pyridin-2-yl)-N-(4-(3,3- dimethylureido)-3- fluorobenzyl)-3-ethyl-5- methyl-5,6,7,8- tetrahydroimidazo[1,5-a] pyrazine-1-carboxamide | 551.3 |
| E261A * | Example 127 | 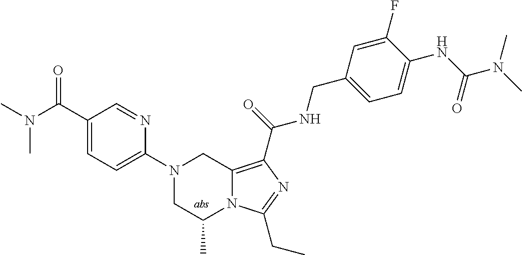 | (R)-7-(5- (dimethylcarbamoyl) pyridin-2-yl)-N-(4-(3,3- dimethylureido)-3- fluorobenzyl)-3-ethyl-5- methyl-5,6,7,8- tetrahydroimidazo[1,5-a] pyrazine-1-carboxamide | 551.4 |
| E262A | Example 127 | 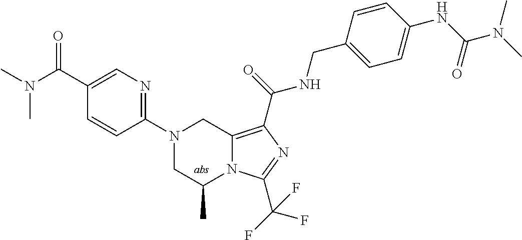 | (S)-7-(5- (dimethylcarbamoyl) pyridin-2-yl)-N-(4-(3,3- dimethylureido)benzyl)- 5-methyl-3- (trifluoromethyl)- 5,6,7,8- tetrahydroimidazo[1,5-a] pyrazine-1-carboxamide | 573.3 |
| E263A | Example 127 | 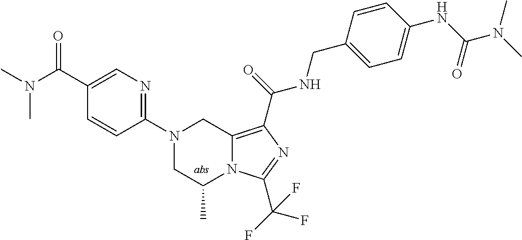 | (R)-7-(5- (dimethylcarbamoyl) pyridin-2-yl)-N-(4-(3,3- dimethyureido)benzyl)- 5-methyl-3- (trifluoromethyl)- 5,6,7,8- tetrahydroimidazo[1,5-a] pyrazine-1-carboxamide | 573.3 |
| E264A * | Example 127 | 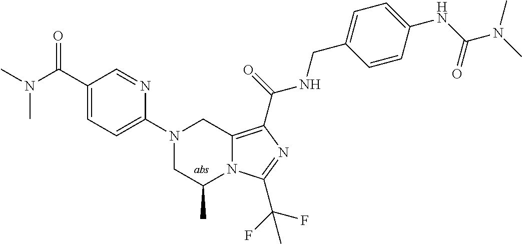 | (S)-3-(1,1- difluoroethyl)-7-(5- (dimethylcarbamoyl) pyridin-2-yl)-N-(4-(3,3- dimethylureido)benzyl)- 5-methyl-5,6,7,8- tetrahydroimidazo[1,5-a] pyrazine-1-carboxamide | 569.3 |
| E265A * | Example 127 | 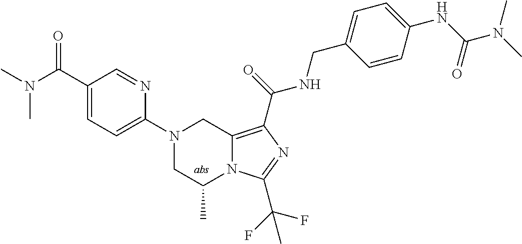 | (R)-3-(1,1- difluoroethyl)-7-(5- (dimethylcarbamoyl) pyridin-2-yl)-N-(4-(3,3- dimethylureido)benzyl)- 5-methyl-5,6,7,8- tetrahydroimidazo[1,5-a] pyrazine-1-carboxamide | 569.3 |
| E266A * | Example 127 | 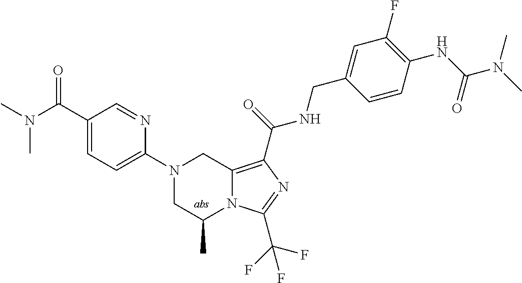 | (S)-7-(5- (dimethylcarbamoyl) pyridin-2-yl)-N-(4-(3,3- dimethylureido)-3- fluorobenzyl)-5-methyl- 3-(trifluoromethyl)- 5,6,7,8- tetrahydroimidazo[1,5-a] pyrazine-1-carboxamide | 591.3 |
| E267A * | Example 127 | 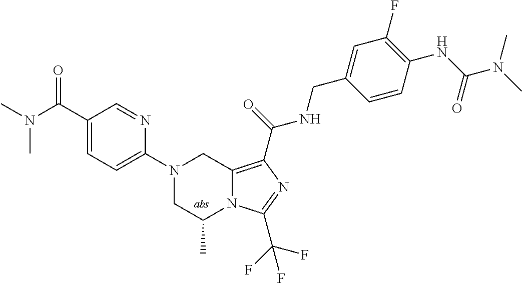 | (R)-7-(5- (dimethylcarbamoyl) pyridin-2-yl)-N-(4-(3,3- dimethylureido)-3- fluorobenzyl)-5-methyl- 3-(trifluoromethyl)- 5,6,7,8- tetrahydroimidazo[1,5-a] pyrazine-1-carboxamide | 591.2 |
| E268A * | Example 127 | 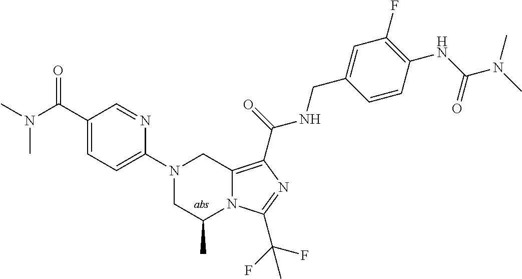 | (S)-3-(1,1- difluoroethyl)-7-(5- (dimethylcarbamoyl) pyridin-2-yl)-N-(4-(3,3- dimethylureido)-3- fluorobenzyl)-5-methyl- 5,6,7,8- tetrahydroimidazo[1,5-a] pyrazine-1-carboxamide | 587.3 |
| E269A * | Example 127 | 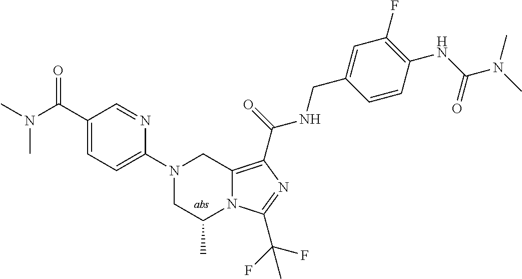 | (R)-3-(1,1- difluoroethyl)-7-(5- (dimethylcarbamoyl) pyridin-2-yl)-N-(4-(3,3- dimethylureido)-3- fluorobenzyl)-5-methyl- 5,6,7,8- tetrahydroimidazo[1,5-a] pyrazine-1-carboxamide | 587.3 |
| E270A | Example 567 | 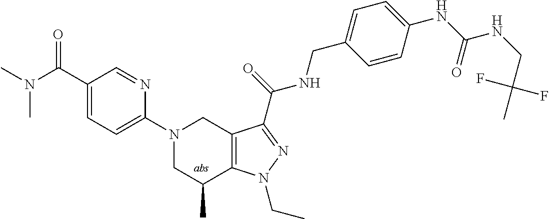 [SMILES: CCn1nc(C(=O)NCc2ccc(NC(=O)NCC(C)(F)F)cc2)c2c1[C@@H](C)CN(c1ccc(C(=O)N(C)C)cn1)C2] | (S)-N-(4-(3-(2,2- difluoropropyl)ureido) benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 583.5 |
| E271A | Example 567 | 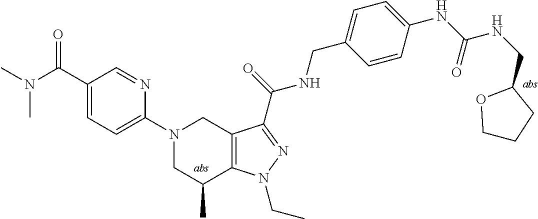 [SMILES: CCn1nc(C(=O)NCc2ccc(NC(=O)NC[C@H]3CCCO3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((R)- tetrahydrofuran-2- yl)methyl)ureido)benzyl)- 4,5,6,7-tetrahydro-1H- pyrazolo[4,3-c]pyridine- 3-carboxamide | 589.5 |
| E272A | Example 567 | 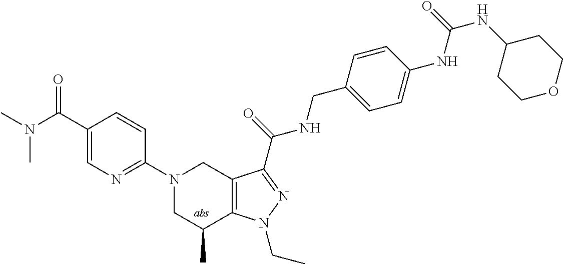 [SMILES: CCn1nc(C(=O)NCc2ccc(NC(=O)NC3CCOCC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3- (tetrahydro-2H-pyran-4- yl)ureido)benzyl)- 4,5,6,7-tetrahydro-1H- pyrazolo[4,3-c]pyridine- 3-carboxamide | 589.5 |
| E273A | Example 567 | 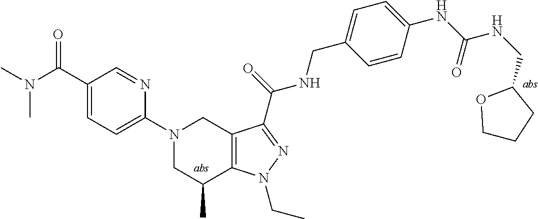 [SMILES: CCn1nc(C(=O)NCc2ccc(NC(=O)NC[C@@H]3CCCO3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((S)- tetrahydrofuran-2- yl)methyl)ureido)benzyl)- 4,5,6,7-tetrahydro-1H- pyrazolo[4,3-c]pyridine- 3-carboxamide | 589.5 |
| E274 | Example 567 | 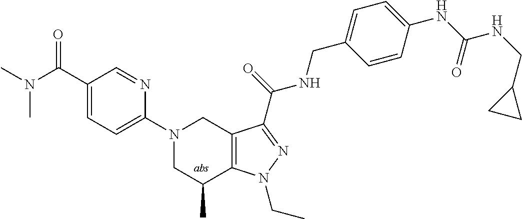 [SMILES: CCn1nc(C(=O)NCc2ccc(NC(=O)NCC3CC3)cc2)c2c1[C@@H](C)CN(c1ccc(C(=O)N(C)C)cn1)C2] | (S)-N-(4-(3- (cyclopropylmethyl) ureido)benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 559.5 |
| E275A | Example 567 | 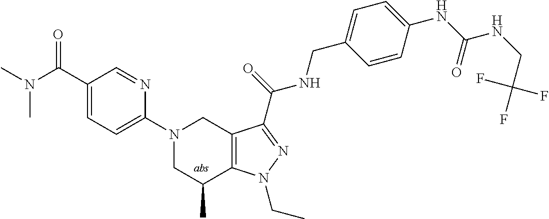 [SMILES: CCn1nc(C(=O)NCc2ccc(NC(=O)NCC(F)(F)F)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(2,2,2- trifluoroethyl)ureido) benzyl)-4,5,6,7-tetrahydro-1H- pyrazolo[4,3-c]pyridine- 3-carboxamide | 587.5 |
| E276A | Example 567 | 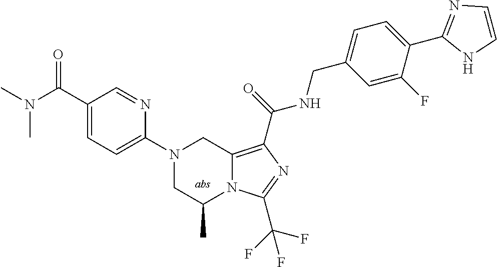 [SMILES: C[C@H]1CN(c2ccc(C(=O)N(C)C)cn2)Cc2c(C(=O)NCc3ccc(-c4ncc[nH]4)c(F)c3)nc(C(F)(F)F)n21] | (S)-7-(5- (dimethylcarbamoyl) pyridin-2-yl)-N-(3-fluoro-4- (1H-imidazol-2- yl)benzyl)-5-methyl-3- (trifluoromethyl)- 5,6,7,8- tetrahydroimidazo[1,5-a] pyrazine-1--carboxamide | 571.2 |
| E277A | Example 567 | 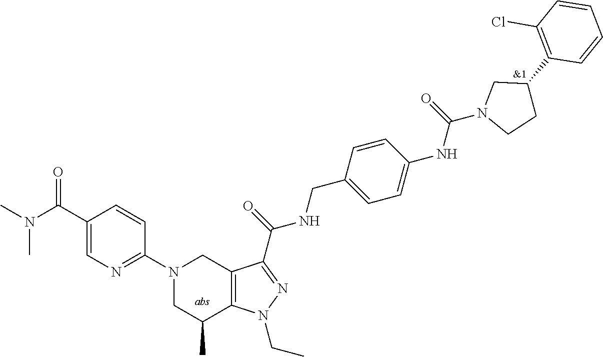 [SMILES: CCn1nc(C(=O)NCc2ccc(NC(=O)N3CC[C@@H](c4ccccc4Cl)C3)cc2)c2c1[C@@H](C)CN(c1ccc(C(=O)N(C)C)cn1)C2] | (S)-N-(4-((RS)-3-(2- chlorophenyl)pyrrolidine-1- carboxamido)benzyl)-5- (5- (dimethylcarbamoyl) pyridin-2-yl)-1-ethyl-7- methyl-4,5,6,7-tetrahydro-1H- pyrazolo[4,3-c]pyridine- 3-carboxamide | 669.5 |
| E278A | Example 567 | 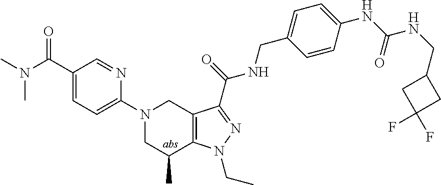 [SMILES: CCn1nc(C(=O)NCc2ccc(NC(=O)NCC3CC(F)(F)C3)cc2)c2c1[C@@H](C)CN(c1ccc(C(=O)N(C)C)cn1)C2] | (S)-N-(4-(3-((3,3- difluorocyclobutyl) methyl)ureido)benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 609.5 |
| E279A | Example 567 | 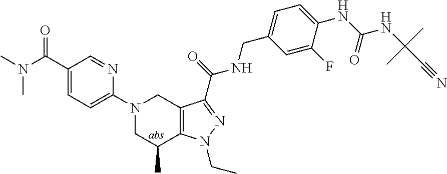 [SMILES: CCn1nc(C(=O)NCc2ccc(NC(=O)NC(C)(C)C#N)c(F)c2)c2c1[C@@H](C)CN(c1ccc(C(=O)N(C)C)cn1)C2] | (S)-N-(4-(3-(2- cyanopropan-2- yl)ureido)-3- fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 590.3 |
| E280A * | Example 127 | 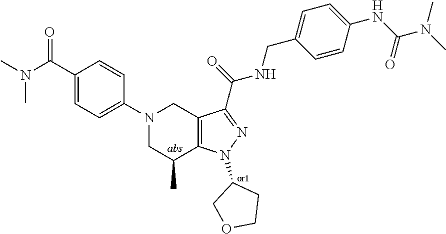 | (S)-5-(4- (dimethylcarbamoyl) phenyl)-N-(4-(3,3- dimethylureido)benzyl)- 7-methyl-1-((R or S*)- tetrahydrofuran-3-yl)- 4,5,6,7-tetrahydro-1H- pyrazolo[4,3-c]pyridine- 3-carboxamide | 574.3 |
| E281A * | Example 127^{c} | 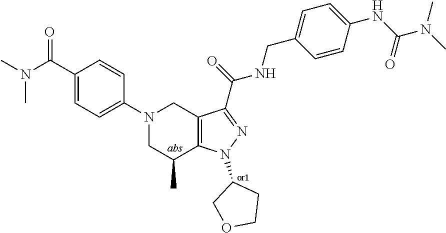 | (S)-5-(4- (dimethylcarbamoyl) phenyl)-N-(4-(3,3- dimethylureido)benzyl)- 7-methyl-1-((R or S*)- tetrahydrofuran-3-yl)- 4,5,6,7-tetrahydro-1H- pyrazolo[4,3-c]pyridine- 3-carboxamide | 574.3 |
| E282A * | Example 132 | 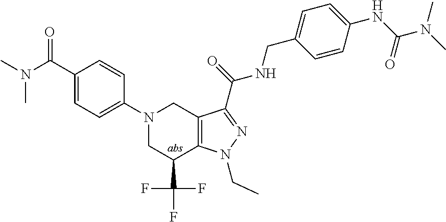 | (S)-5-(4- (dimethylcarbamoyl) phenyl)-N-(4-(3,3- dimethylureido)benzyl)- 1-ethyl-7- (trifluoromethyl)- 4,5,6,7-tetrahydro-1H- pyrazolo[4,3-c]pyridine- 3-carboxamide | 586.2 |
| E283A * | Example 132 | 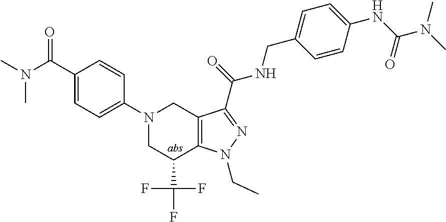 | (R)-5-(4- (dimethylcarbamoyl) phenyl)-N-(4-(3,3- dimethylureido)benzyl)- 1-ethyl-7- (trifluoromethyl)- 4,5,6,7-tetrahydro-1H- pyrazolo[4,3-c]pyridine- 3-carboxamide | 586.3 |
| E284A | Example 567 | 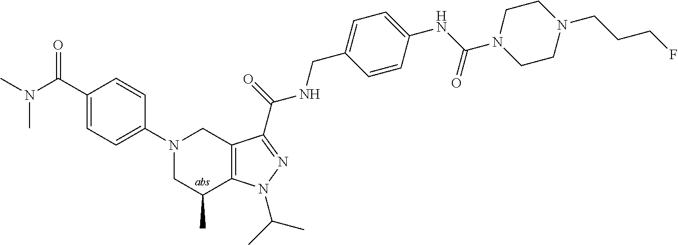 [SMILES: CC(C)n1nc(C(=O)NCc2ccc(NC(=O)N3CCN(CCCF)CC3)cc2)c2c1[C@@H](C)CN(c1ccc(C(=O)N(C)C)cc1)C2] | (S)-5-(4- (dimethylcarbamoyl) phenyl)-N-(4-(4-(3- fluoropropyl)piperazine- 1-carboxamido)benzyl)- 1-isopropyl-7-methyl- 4,5,6,7-tetrahydro-1H- pyrazolo[4,3-c]pyridine- 3-carboxamide | 647.4 |
| E285A | Example 126 | 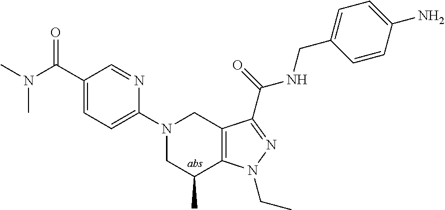 [SMILES: CCn1nc(C(=O)NCc2ccc(N)cc2)c2c1[C@@H](C)CN(c1ccc(C(=O)N(C)C)cn1)C2] | (S)-N-(4-aminobenzyl)- 5-(5- (dimethylcarbamoyl) pyridin-2-yl)-1-ethyl-7- methyl-4,5,6,7-tetrahydro-1H- pyrazolo[4,3-c]pyridine- 3-carboxamide | 462.1 |
| E286A | Example 567 | 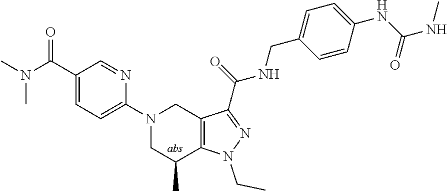 | (S)-5-(5- (dimethylcarbamoyl) pyridin-2-yl)-1-ethyl-7- methyl-N-(4-(3- methylureido)benzyl)- 4,5,6,7-tetrahydro-1H- pyrazolo[4,3-c]pyridine- 3-carboxamide | 519.3 |
| E287A | Example 236 | 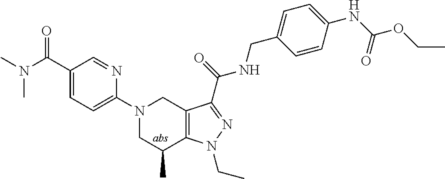 [SMILES: CCOC(=O)Nc1ccc(CNC(=O)c2nn(CC)c3c2CN(c2ccc(C(=O)N(C)C)cn2)C[C@@H]3C)cc1] | ethyl (S)-(4-((5-(5- (dimethylcarbamoyl) pyridin-2-yl)-1-ethyl-7- methyl-4,5,6,7-tetrahydro-1H- pyrazolo[4,3-c]pyridine- 3-carboxamido)methyl)phenyl) carbamate | 534.3 |
| E288A | Example 127 | 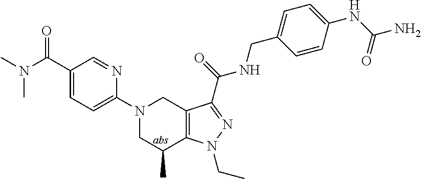 [SMILES: CCn1nc(C(=O)NCc2ccc(NC(N)=O)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4- ureidobenzyl)-4,5,6,7- tetrahydro-1H- pyrazolo[4,3-c]pyridine- 3-carboxamide | 505.4 |
| E289A | Example 127^{c} | 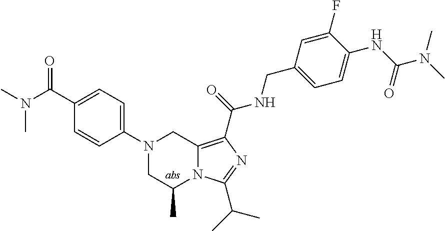 | (S)-7-(4- (dimethylcarbamoyl) phenyl)-N-(4-(3,3- dimethylureido)-3- fluorobenzyl)-3- isopropyl-5-methyl- 5,6,7,8- tetrahydroimidazo[1,5- a]pyrazine-1-carboxamide | 564.5 |
| E290A | Example 127 | 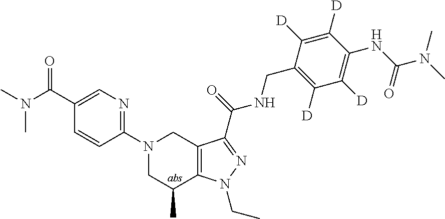 | (S)-5-(5- (dimethylcarbamoyl) pyridin-2-yl)-N-(4-(3,3- dimethylureido)- 2,3,5,6- tetradeutrobenzyl)-1- ethyl-7-methyl-4,5,6,7- tetrahydro-1H- pyrazolo[4,3-c]pyridine- 3-carboxamide | 537.3 |
| E291A | Example 127 | 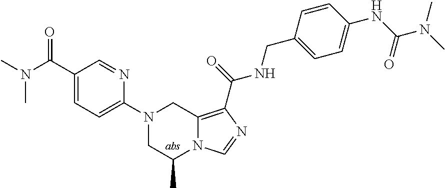 | (S)-7-(5- (dimethylcarbamoyl) pyridin-2-yl)-N-(4-(3,3- dimethylureido)benzyl)- 5-methyl-5,6,7,8- tetrahydroimidazo[1,5- a]pyrazine-1-carboxamide | 505.4 |
| E292A | Example 9 | 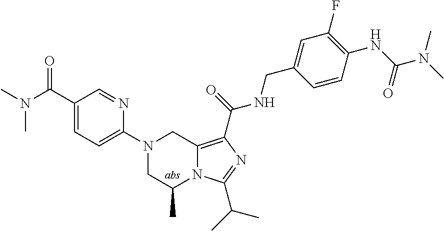 | (S)-7-(5- (dimethylcarbamoyl) pyridin-2-yl)-N-(4-(3,3- dimethylureido)-3- fluorobenzyl)-3- isopropyl-5-methyl- 5,6,7,8- tetrahydroimidazo[1,5- a]pyrazine-1-carboxamide | 565.5 |
| E293A | Example 9 | 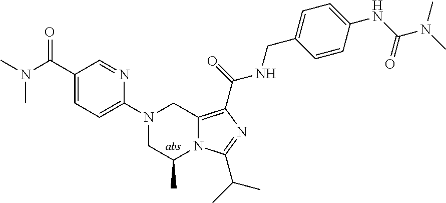 | (S)-7-(5- (dimethylcarbamoyl) pyridin-2-yl)-N-(4-(3,3- dimethylureido)benzyl)- 3-isopropyl-5-methyl- 5,6,7,8- tetrahydroimidazo[1,5- a]pyrazine-1-carboxamide | 547.5 |
| E294A | Example 126^{c} | 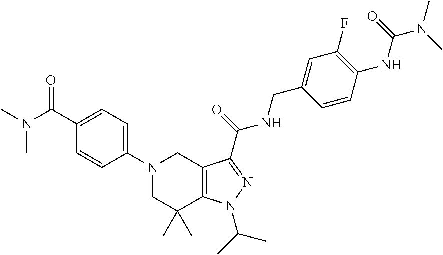 | 5-(4- (dimethylcarbamoyl) phenyl)-N-(4-(3,3- dimethylureido)-3- fluorobenzyl)-1- isopropyl-7,7-dimethyl- 4,5,6,7- tetrahydro-1H- pyrazolo[4,3-c]pyridine- 3-carboxamide | 578.5 |
| E295A | Example 395 | 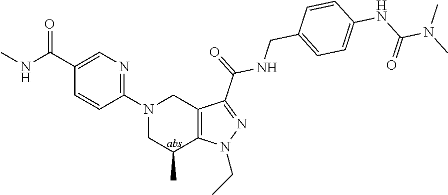 | (S)-N-(4-(3,3- dimethylureido)benzyl)- 1-ethyl-7-methyl-5-(5- (methylcarbamoyl) pyridin-2-yl)-4,5,6,7- tetrahydro-1H- pyrazolo[4,3-c]pyridine- 3-carboxamide | 519.4 |
| E296A * | Example 537 | 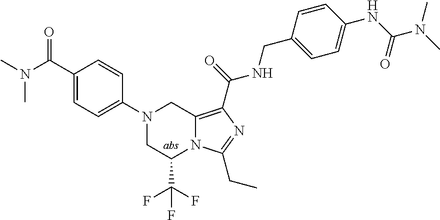 | (S)-7-(4- (dimethylcarbamoyl) phenyl)-N-(4-(3,3- dimethylureido)benzyl)- 3-ethyl-5- (trifluoromethyl)-5,6,7,8- tetrahydroimidazo[1,5-a] pyrazine-1-carboxamide | 586.3 |
| E297A * | Example 537 | 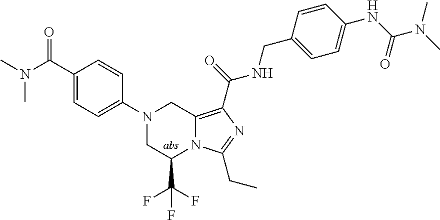 | (RS)-7-(4- (dimethylcarbamoyl) phenyl)-N-(4-(3,3- dimethylureido)benzyl)- 3-ethyl-5- (trifluoromethyl)- 5,6,7,8-tetrahydroimidazo [1,5-a]pyrazine-1-carboxamide | 586.3 |
| E298A | Example 567 | 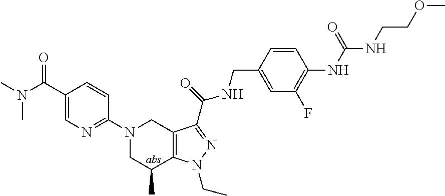 | (S)-5-(5- (dimethylcarbamoyl) pyridin-2-yl)-1-ethyl-N-(3- fluoro-4-(3-(2- methoxyethyl)ureido) benzyl)-7-methyl- 4,5,6,7- tetrahydro-1H- pyrazolo[4,3-c]pyridine- 3-carboxamide | 581.4 |
| E299A | Example 567 | 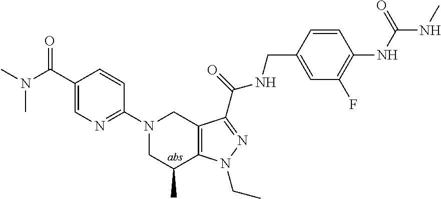 | (S)-5-(5- (dimethylcarbamoyl) pyridin-2-yl)-1-ethyl-N-(3- fluoro-4-(3- methylureido)benzyl)-7- methyl-4,5,6,7- tetrahydro-1H- pyrazolo[4,3-c]pyridine- 3-carboxamide | 537.3 |
| E300A | Example 567 | 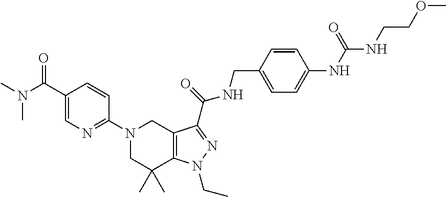 | 5-(5- (dimethylcarbamoyl) pyridin-2-yl)-1-ethyl-N-(4- (3-(2- methoxyethyl)ureido) benzyl)-7,7-dimethyl- 4,5,6,7- tetrahydro-1H- pyrazolo[4,3-c]pyridine- 3-carboxamide | 577.4 |
| E301A | Example 498 | 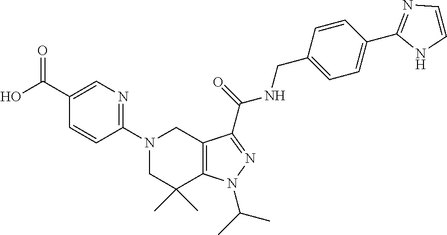 [SMILES: CC(C)n1nc(C(=O)NCc2ccc(-c3ncc[nH]3)cc2)c2c1C(C)(C)CN(c1ccc(C(=O)O)cn1)C2] | 6-(3-((4-(1H-imidazol-2- yl)benzyl)carbamoyl)-1- isopropyl-7,7-dimethyl- 1,4,6,7-tetrahydro-5H- pyrazolo[4,3-c]pyridin- 5-yl)nicotinic acid | 540.4 |
| E302A | Example 567 | 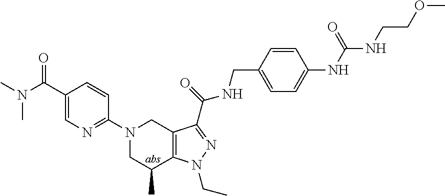 | (S)-5-(5- (dimethylcarbamoyl) pyridin-2-yl)-1-ethyl-N-(4- (3-(2- methoxyethyl)ureido) benzyl)-7-methyl- 4,5,6,7- tetrahydro-1H- pyrazolo[4,3-c]pyridine- 3-carboxamide | 563.5 |
| E303A | Example 567 | 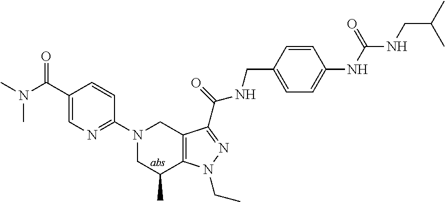 [SMILES: CCn1nc(C(=O)NCc2ccc(NC(=O)NCC(C)C)cc2)c2c1[C@@H](C)CN(c1ccc(C(=O)N(C)C)cn1)C2] | (S)-5-(5- (dimethylcarbamoyl) pyridin-2-yl)-1-ethyl-N-(4-(3- isobutylureido)benzyl)- 7-methyl-4,5,6,7- tetrahydro-1H- pyrazolo[4,3-c]pyridine- 3-carboxamide | 540.4 |
| E304A | Example 567 | 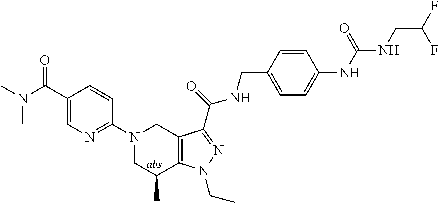 [SMILES: CCn1nc(C(=O)NCc2ccc(NC(=O)NCC(F)F)cc2)c2c1[C@@H](C)CN(c1ccc(C(=O)N(C)C)cn1)C2] | (S)-N-(4-(3-(2,2- difluoroethyl)ureido) benzyl)-5-(5- (dimethylcarbamoyl) pyridin-2-yl)-1-ethyl-7- methyl-4,5,6,7- tetrahydro-1H- pyrazolo[4,3-c]pyridine- 3-carboxamide | 539.4 |
| E305A | Example 567 | 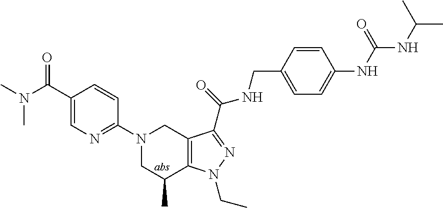 [SMILES: CCn1nc(C(=O)NCc2ccc(NC(=O)NC(C)C)cc2)c2c1[C@@H](C)CN(c1ccc(C(=O)N(C)C)cn1)C2] | (S)-5-(5- (dimethylcarbamoyl) pyridin-2-yl)-1-ethyl-N-(4- (3-isopropylureido)benzyl)- 7-methyl-4,5,6,7- tetrahydro-1H- pyrazolo[4,3-c]pyridine- 3-carboxamide | 547.5 |
| E306A | Example 567 | 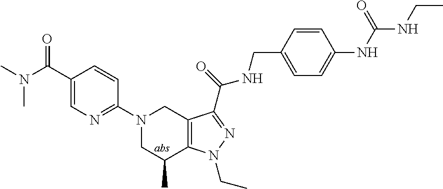 [SMILES: CCNC(=O)Nc1ccc(CNC(=O)c2nn(CC)c3c2CN(c2ccc(C(=O)N(C)C)cn2)C[C@@H]3C)cc1] | (S)-5-(5- (dimethylcarbamoyl) pyridin-2-yl)-1-ethyl-N-(4- (3-ethylureido)benzyl)- 7-methyl-4,5,6,7- tetrahydro-1H- pyrazolo[4,3-c]pyridine- 3-carboxamide | 533.5 |
| E307A | Example 567 | 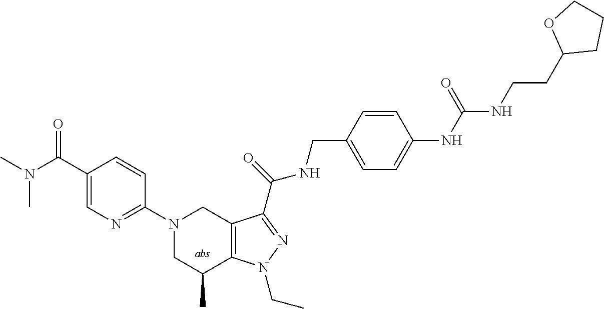 [SMILES: CCn1nc(C(=O)NCc2ccc(NC(=O)NCCC3CCCO3)cc2)c2c1[C@@H](C)CN(c1ccc(C(=O)N(C)C)cn1)C2] | (7S)-5-(5- (dimethylcarbamoyl) pyridin-2-yl)-1-ethyl-7- methyl-N-(4-(3-(2- (tetrahydrofuran-2- yl)ethyl)ureido)benzyl)- 4,5,6,7- tetrahydro-1H- pyrazolo[4,3-c]pyridine- 3-carboxamide | 603.5 |
| E308A | Example 567 | 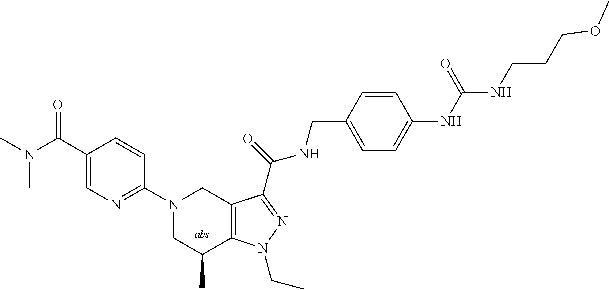 | (S)-5-(5- (dimethylcarbamoyl) pyridin-2-yl)-1-ethyl-N-(4- (3-(3- methoxypropyl)ureido) benzyl)-7-methyl- 4,5,6,7-tetrahydro-1H- pyrazolo[4,3-c]pyridine- 3-carboxamide | 577.5 |
| E309A | Example 537 | 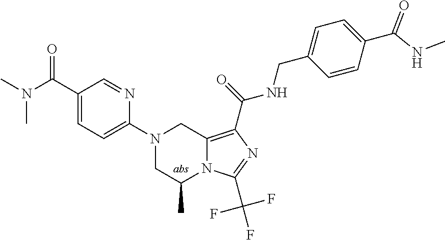 | (S)-7-(5- (dimethylcarbamoyl) pyridin-2-yl)-5-methyl-N- (4- (methylcarbamoyl)benzy)- 3-(trifluoromethyl)- 5,6,7,8- tetrahydroimidazo[1,5-a] pyrazine-1-carboxamide | 544.4 |
| E310A | Example 126^{d} | 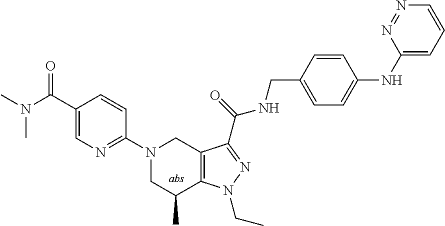 [SMILES: CCn1nc(C(=O)NCc2ccc(Nc3cccnn3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(pyridazin- 3-ylamino)benzyl)- 4,5,6,7-tetrahydro-1H- pyrazolo[4,3-c]pyridine- 3-carboxamide | 540.4 |
| E311A | Example 126^{d} | 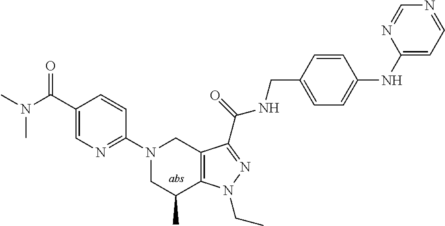 [SMILES: CCn1nc(C(=O)NCc2ccc(Nc3ccncn3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(pyrimidin- 4-ylamino)benzyl)- 4,5,6,7-tetrahydro-1H- pyrazolo[4,3-c]pyridine- 3-carboxamide | 540.4 |
| E312A | Example 126^{d} | 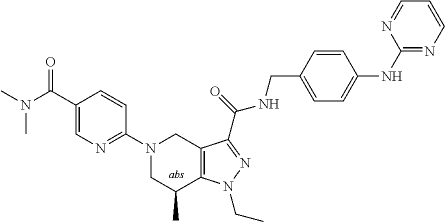 [SMILES: CCn1nc(C(=O)NCc2ccc(Nc3ncccn3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(pyrimidin- 2-ylamino)benzyl)- 4,5,6,7-tetrahydro-1H- pyrazolo[4,3-c]pyridine- 3-carboxamide | 540.4 |
| E313A | Example 126^{d} | 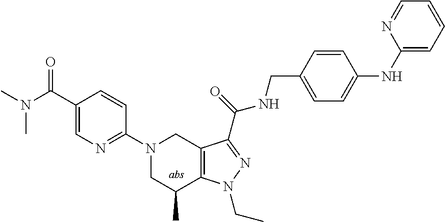 [SMILES: CCn1nc(C(=O)NCc2ccc(Nc3ccccn3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(pyridin-2- ylamino)benzyl)- 4,5,6,7-tetrahydro-1H- pyrazolo[4,3-c]pyridine- 3-carboxamide | 539.4 |
| E314A | Example 126^{d} | 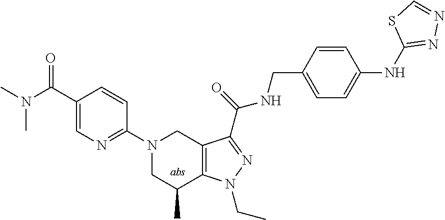 [SMILES: CCn1nc(C(=O)NCc2ccc(Nc3nncs3)cc2)c2c1[C@@H](C)CN(c1ccc(C(=O)N(C)C)cn1)C2] | (S)-N-(4-((1,3,4- thiadiazol-2- yl)amino)benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 546.4 |
| E315A | Example 126^{d} | 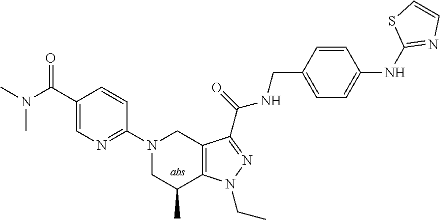 [SMILES: CCn1nc(C(=O)NCc2ccc(Nc3nccs3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(thiazol-2- ylamino)benzyl)- 4,5,6,7-tetrahydro-1H- pyrazolo[4,3-c]pyridine- 3-carboxamide | 545.4 |
| E316A | Example 126^{d} | 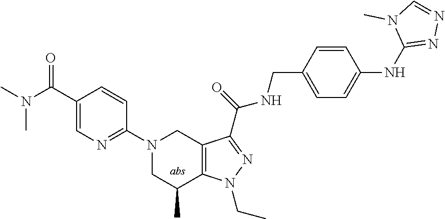 [SMILES: CCn1nc(C(=O)NCc2ccc(Nc3nncn3C)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-((4-methyl- 4H-1,2,4-triazol-3- yl)amino)benzyl)- 4,5,6,7-tetrahydro-1H- pyrazolo[4,3-c]pyridine- 3-carboxamide | 543.5 |
| E317A | Example 567 | 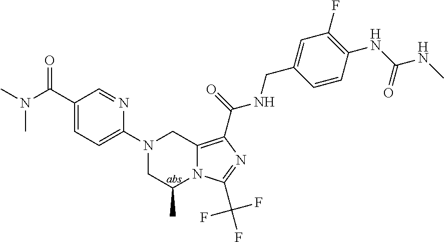 | (S)-7-(5- (dimethylcarbamoyl) pyridin-2-yl)-N-(3-fluoro-4- (3- methylureido)benzyl)-5- methyl-3- (trifluoromethyl)-5,6,7,8- tetrahydroimidazo[1,5-a] pyrazine-1-carboxamide | 577.4 |
| E318A | Example 567 | 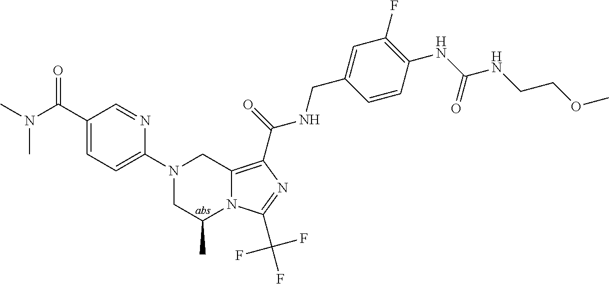 | (S)-7-(5- (dimethylcarbamoyl) pyridin-2-yl)-N-(3-fluoro-4- (3-(2- methoxyethyl)ureido) benzyl)-5-methyl-3- (trifluoromethyl)- 5,6,7,8- tetrahydroimidazo[1,5-a] pyrazine-1-carboxamide | 621.5 |
| E319A | Example 567 | 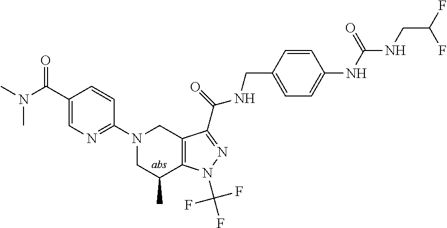 [SMILES: C[C@H]1CN(c2ccc(C(=O)N(C)C)cn2)Cc2c(C(=O)NCc3ccc(NC(=O)NCC(F)F)cc3)nn(C(F)(F)F)c21] | (S)-N-(4-(3-(2,2- difluoroethyl)ureido) benzyl)-7-(5- (dimethylcarbamoyl) pyridin-2-yl)-5-methyl-3- (trifluoromethyl)- 5,6,7,8- tetrahydroimidazo[1,5-a] pyrazine-1-carboxamide | 609.3 |
| E320A | Example 567 | 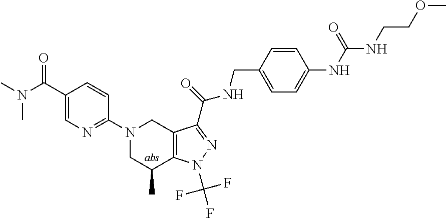 | (S)-7-(5- (dimethylcarbamoyl) pyridin-2-yl)-N-(4-(3-(2- methoxyethyl)ureido) benzyl)-5-methyl-3- (trifluoromethyl)- 5,6,7,8- tetrahydroimidazo[1,5-a] pyrazine-1-carboxamide | 603.3 |
| E321A | Example 567 | 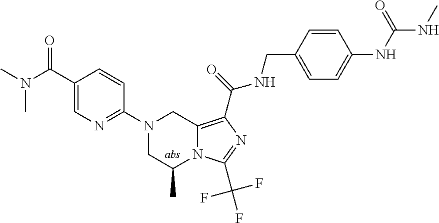 | (S)-7-(5- (dimethylcarbamoyl) pyridin-2-yl)-5-methyl-N- (4-(3- methylureido)benzyl)-3- (trifluoromethyl)- 5,6,7,8- tetrahydroimidazo[1,5-a] pyrazine-1-carboxamide | 559.3 |
| E322A | Example 567 | 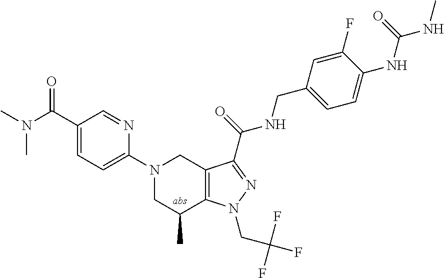 | (S)-5-(5- (dimethylcarbamoyl) pyridin-2-yl)-N-(3-fluoro-4- (3- methylureido)benzyl)-7- methyl-1-(2,2,2- trifluoroethyl)- 4,5,6,7-tetrahydro-1H- pyrazolo[4,3-c]pyridine- 3-carboxamide | 591.3 |
| E323A | Example 567 | 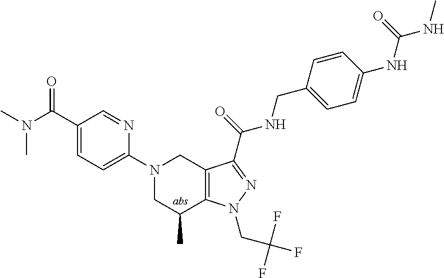 | (S)-5-(5- (dimethylcarbamoyl) pyridin-2-yl)-7-methyl-N- (4-(3- methylureido)benzyl)-1- (2,2,2-trifluoroethyl)- 4,5,6,7-tetrahydro-1H- pyrazolo[4,3-c]pyridine- 3-carboxamide | 573.4 |
| E324A | Example 567 | 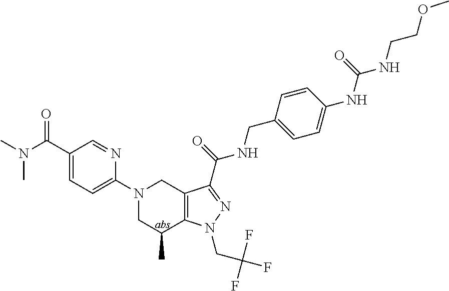 | (S)-5-(5- (dimethylcarbamoyl) pyridin-2-yl)-N-(4-(3-(2- methoxyethyl)ureido) benzyl)-7-methyl-1-(2,2,2- trifluoroethyl)- 4,5,6,7-tetrahydro-1H- pyrazolo[4,3-c]pyridine- 3-carboxamide | 617.5 |
| E325A | Example 126 | 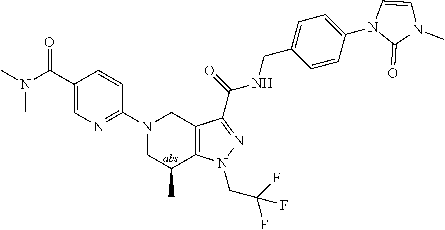 | (S)-5-(5- (dimethylcarbamoyl) pyridin-2-yl)-7-methyl-N- (4-(3-methyl-2-oxo-2,3- dihydro-1H-imidazol-1- yl)benzyl)-1-(2,2,2- trifluoroethyl)- 4,5,6,7-tetrahydro-1H- pyrazolo[4,3-c]pyridine- 3-carboxamide | 597.3 |
| E326A | Example 126 | 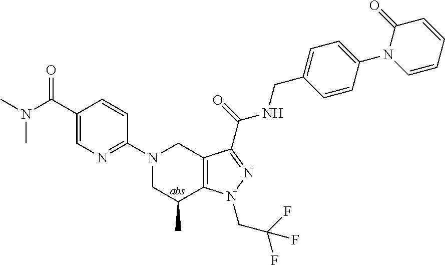 [SMILES: C[C@H]1CN(c2ccc(C(=O)N(C)C)cn2)Cc2c(C(=O)NCc3ccc(-n4ccccc4=O)cc3)nn(CC(F)(F)F)c21] | (S)-5-(5- (dimethylcarbamoyl) pyridin-2-yl)-7-methyl-N- (4-(2-oxopyridin-1(2H)- yl)benzyl)-1-(2,2,2- trifluoroethyl)- 4,5,6,7-tetrahydro-1H- pyrazolo[4,3-c]pyridine- 3-carboxamide | 594.3 |
| E327A | Example 126 | 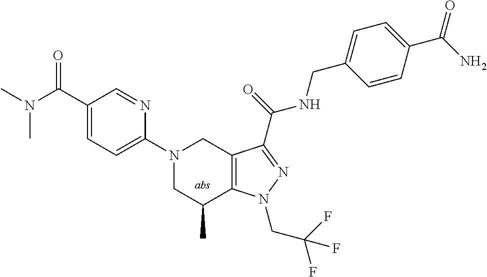 [SMILES: C[C@H]1CN(c2ccc(C(=O)N(C)C)cn2)Cc2c(C(=O)NCc3ccc(C(N)=O)cc3)nn(CC(F)(F)F)c21] | (S)-N-(4- carbamoylbenzyl)-5-(5- (dimethylcarbamoyl) pyridin-2-yl)-7-methyl-1- (2,2,2-trifluoroethyl)- 4,5,6,7-tetrahydro-1H- pyrazolo[4,3-c]pyridine- 3-carboxamide | 544.2 |
| E328A | Example 126 | 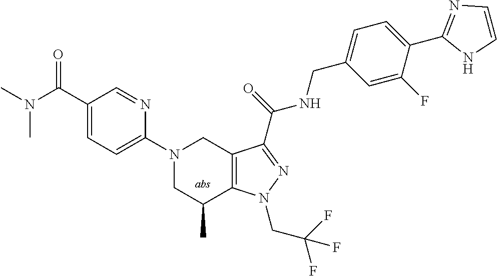 [SMILES: C[C@H]1CN(c2ccc(C(=O)N(C)C)cn2)Cc2c(C(=O)NCc3ccc(-c4ncc[nH]4)c(F)c3)nn(CC(F)(F)F)c21] | (S)-5-(5- (dimethylcarbamoyl) pyridin-2-yl)-N-(3-fluoro-4- (1H-imidazol-2- yl)benzyl)-7-methyl-1- (2,2,2-trifluoroethyl)- 4,5,6,7-tetrahydro-1H- pyrazolo[4,3-c]pyridine- 3-carboxamide | 585.3 |
| E329A | Example 126 | 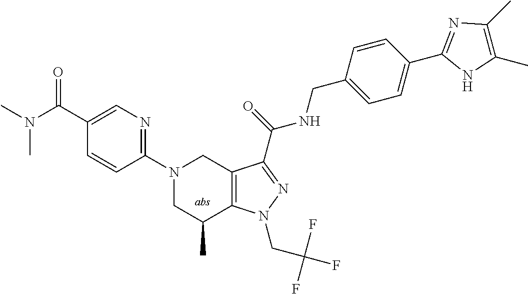 | (S)-N-(4-(4,5-dimethyl- 1H-imidazol-2- yl)benzyl)-5-(5- (dimethylcarbamoyl) pyridin-2-yl)-7-methyl-1- (2,2,2-trifluoroethyl)- 4,5,6,7-tetrahydro-1H- pyrazolo[4,3-c]pyridine- 3-carboxamide | 595.3 |
| E330A | Example 126 | 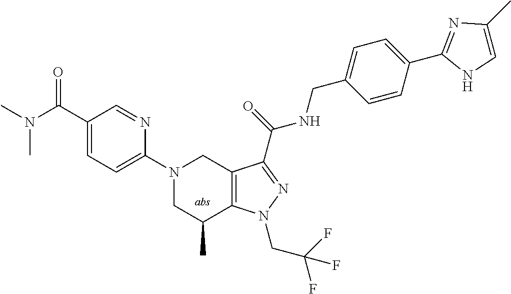 | (S)-5-(5- (dimethylcarbamoyl) pyridin-2-yl)-7-methyl-N- (4-(4-methyl-1H- imidazol-2-yl)benzyl)-1- (2,2,2-trifluoroethyl)- 4,5,6,7-tetrahydro-1H- pyrazolo[4,3-c]pyridine- 3-carboxamide | 581.3 |
| E331A | Example 126 | 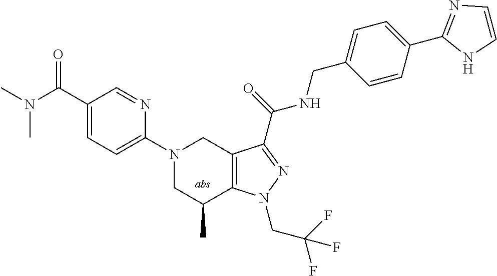 [SMILES: C[C@H]1CN(c2ccc(C(=O)N(C)C)cn2)Cc2c(C(=O)NCc3ccc(-c4ncc[nH]4)cc3)nn(CC(F)(F)F)c21] | (S)-N-(4-(1H-imidazol- 2-yl)benzyl)-5-(5- (dimethylcarbamoyl) pyridin-2-yl)-7-methyl-1- (2,2,2-trifluoroethyl)- 4,5,6,7-tetrahydro-1H- pyrazolo[4,3-c]pyridine- 3-carboxamide | 567.3 |
| E332A | Example 126 | 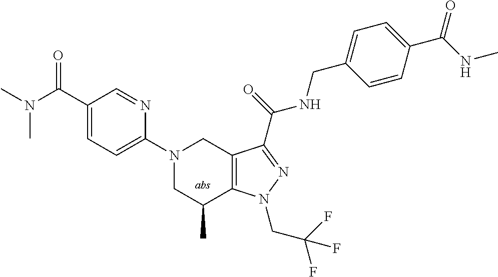 | (S)-5-(5- (dimethylcarbamoyl) pyridin-2-yl)-7-methyl-N-(4- (methylcarbamoyl)benzyl)-1- (2,2,2-trifluoroethyl)- 4,5,6,7-tetrahydro-1H- pyrazolo[4,3-c]pyridine- 3-carboxamide | 558.3 |
| E333A | Example 126 | 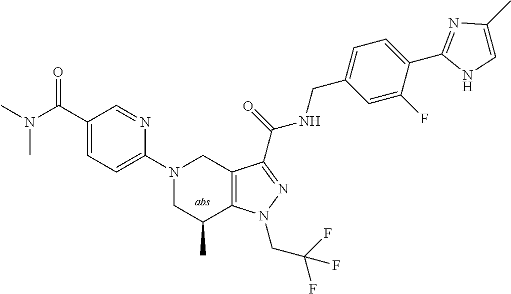 | (S)-5-(5- (dimethylcarbamoyl) pyridin-2-yl)-1-ethyl-N-(3- fluoro-4-(3-(2,2,2- trifluoroethyl)ureido) benzyl)-7-methyl- 4,5,6,7-tetrahydro-1H- pyrazolo[4,3-c]pyridine- 3-carboxamide | 599.3 |
| E334A | Example 567 | 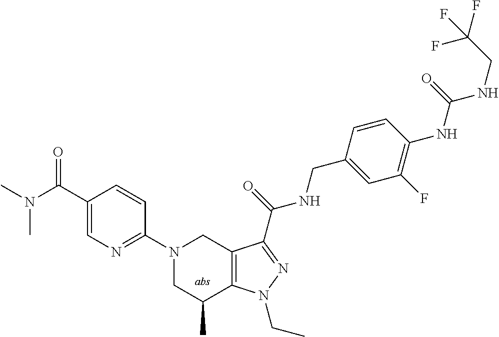 [SMILES: CCn1nc(C(=O)NCc2ccc(NC(=O)NCC(F)(F)F)c(F)c2)c2c1[C@@H](C)CN(c1ccc(C(=O)N(C)C)cn1)C2] | (S)-5-(5- (dimethylcarbamoyl) pyridin-2-yl)-N-(3-fluoro-4- (4-methyl-1H-imidazol- 2-yl)benzyl)-7-methyl- 1-(2,2,2-trifluoroethyl)- 4,5,6,7-tetrahydro-1H- pyrazolo[4,3-c]pyridine- 3-carboxamide | 605.3 |
| E335A | Example 567 | 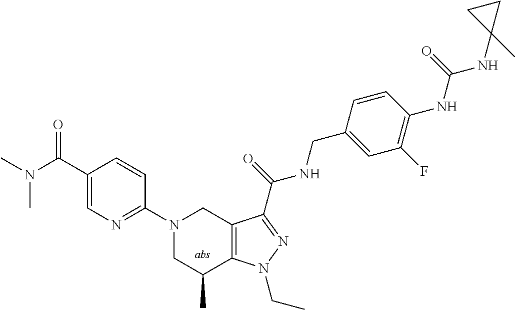 | (S)-5-(5- (dimethylcarbamoyl) pyridin-2-yl)-1-ethyl-N-(3- fluoro-4-(3-(1- methylcyclopropyl)ureido) benzyl)-7-methyl- 4,5,6,7-tetrahydro-1H- pyrazolo[4,3-c]pyridine- 3-carboxamide | 577.4 |
| E336A | Example 567 | 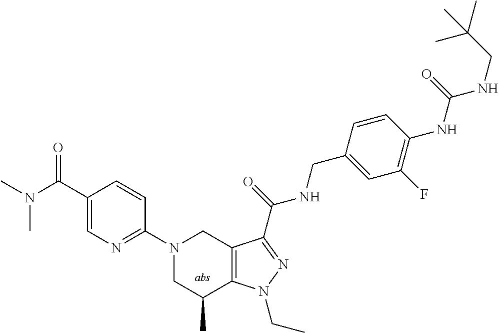 [SMILES: CCn1nc(C(=O)NCc2ccc(NC(=O)NCC(C)(C)C)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3- neopentylureido)benzyl)-7- methyl-4,5,6,7-tetrahydro-1H- pyrazolo[4,3-c]pyridine- 3-carboxamide | 593.4 |
| E337A | Example 567 | 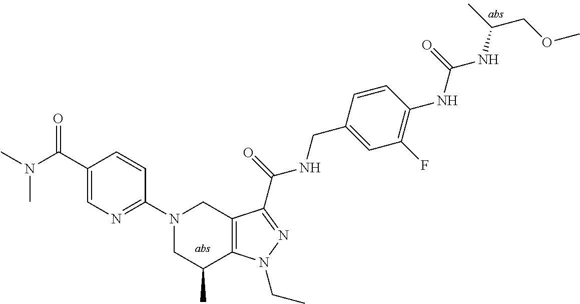 | (S)-5-(5- (dimethylcarbamoyl) pyridin-2-yl)-1-ethyl-N-(3- fluoro-4-(3-((R)-1- methoxypropan-2- yl)ureido)benzyl)-7- methyl-4,5,6,7-tetrahydro-1H- pyrazolo[4,3-c]pyridine- 3-carboxamide | 595.4 |
| E338A | Example 567 | 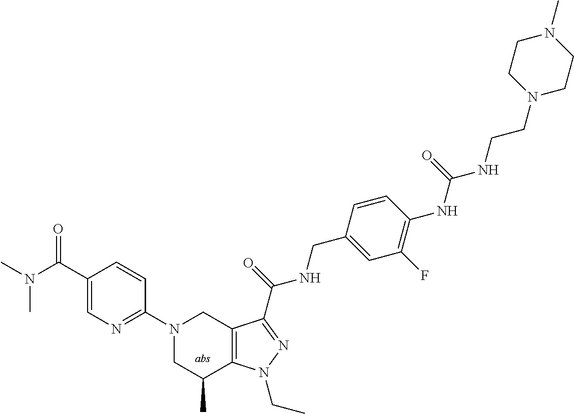 [SMILES: CCn1nc(C(=O)NCc2ccc(NC(=O)NCCN3CCN(C)CC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(2-(4- methylpiperazin-1-yl) ethyl)ureido)benzyl)- 7-methyl- 4,5,6,7-tetrahydro-1H- pyrazolo[4,3-c]pyridine- 3-carboxamide | 649.5 |
| E339A | Example 567 | 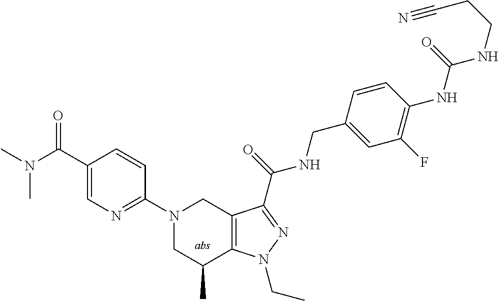 [SMILES: CCn1nc(C(=O)NCc2ccc(NC(=O)NCCC#N)c(F)c2)c2c1[C@@H](C)CN(c1ccc(C(=O)N(C)C)cn1)C2] | (S)-N-(4-(3-(2- cyanoethyl)ureido)-3- fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 576.3 |
| E340A | Example 567 | 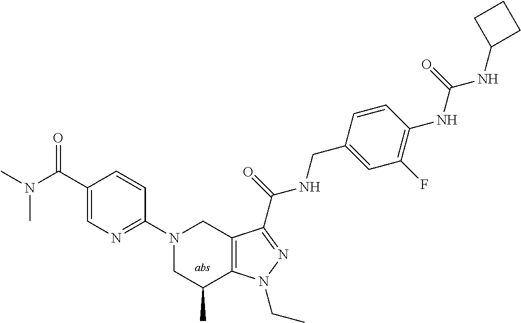 [SMILES: CCn1nc(C(=O)NCc2ccc(NC(=O)NC3CCC3)c(F)c2)c2c1[C@@H](C)CN(c1ccc(C(=O)N(C)C)cn1)C2] | (S)-N-(4-(3- cyclobutylureido)-3- fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 577.3 |
| E341A | Example 567 | 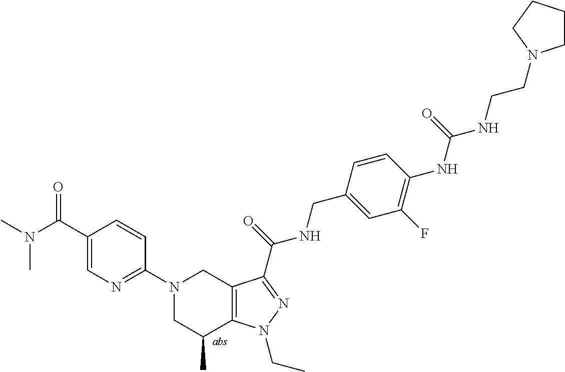 [SMILES: CCn1nc(C(=O)NCc2ccc(NC(=O)NCCN3CCCC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(2- (pyrrolidin-1- yl)ethyl)ureido)benzyl)-7- methyl-4,5,6,7-tetrahydro-1H- pyrazolo[4,3-c]pyridine- 3-carboxamide | 620.4 |
| E342A | Example 567 | 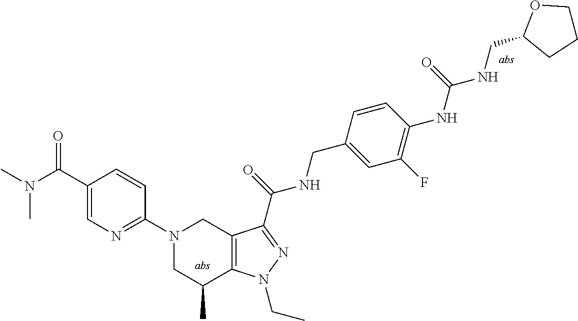 [SMILES: CCn1nc(C(=O)NCc2ccc(NC(=O)NC[C@H]3CCCO3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(((R)- tetrahydrofuran-2- yl)methyl)ureido)benzyl)-7- methyl-4,5,6,7-tetrahydro-1H- pyrazolo[4,3-c]pyridine- 3-carboxamide | 607.4 |
| E343A | Example 567 | 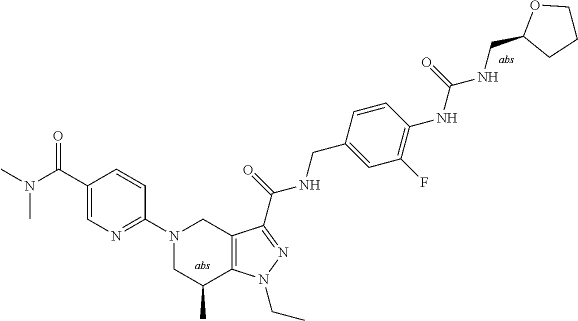 [SMILES: CCn1nc(C(=O)NCc2ccc(NC(=O)NC[C@@H]3CCCO3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(((S)- tetrahydrofuran-2- yl)methyl)ureido)benzyl)-7- methyl-4,5,6,7-tetrahydro-1H- pyrazolo[4,3-c]pyridine- 3-carboxamide | 607.4 |
| E344A | Example 567 | 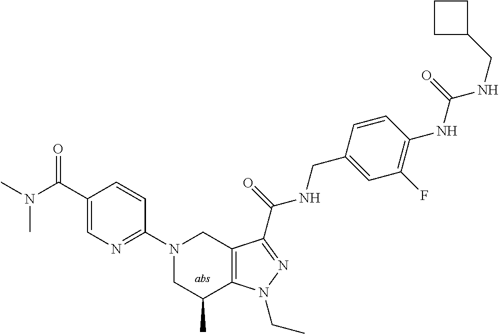 [SMILES: CCn1nc(C(=O)NCc2ccc(NC(=O)NCC3CCC3)c(F)c2)c2c1[C@@H](C)CN(c1ccc(C(=O)N(C)C)cn1)C2] | (S)-N-(4-(3- (cyclobutylmethyl)ureido)- 3-fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 591.4 |
| E345A | Example 567 | 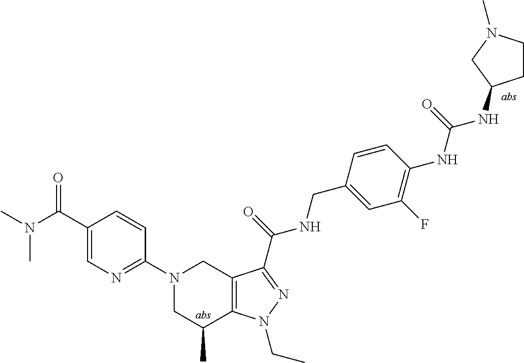 [SMILES: CCn1nc(C(=O)NCc2ccc(NC(=O)N[C@@H]3CCN(C)C3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((R)-1- methylpyrrolidin-3- yl)ureido)benzyl)-7-methyl- 4,5,6,7-tetrahydro-1H- pyrazolo[4,3-c]pyridine- 3-carboxamide | 606.4 |
| E346A | Example 567 | 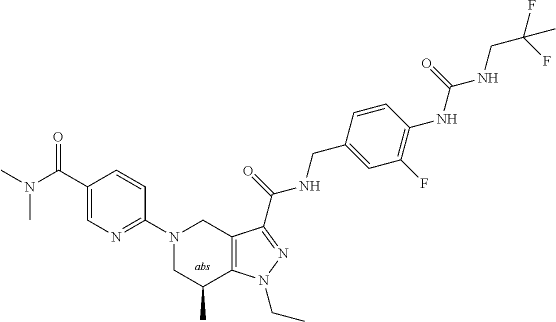 [SMILES: CCn1nc(C(=O)NCc2ccc(NC(=O)NCC(C)(F)F)c(F)c2)c2c1[C@@H](C)CN(c1ccc(C(=O)N(C)C)cn1)C2] | (S)-N-(4-(3-(2,2- difluoropropyl)ureido)- 3-fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 601.3 |
| E347A | Example 567 | 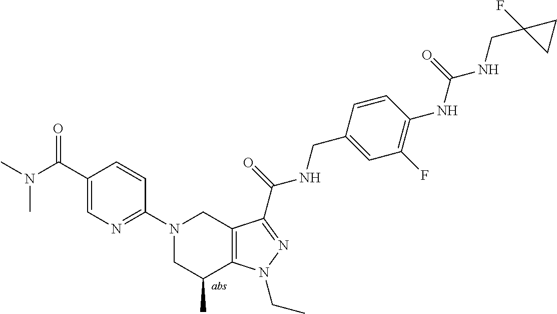 [SMILES: CCn1nc(C(=O)NCc2ccc(NC(=O)NCC3(F)CC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((1- fluorocyclopropyl) methyl)ureido)benzyl)-7- methyl-4,5,6,7-tetrahydro-1H- pyrazolo[4,3-c]pyridine- 3-carboxamide | 595.4 |
| E348A | Example 126 | 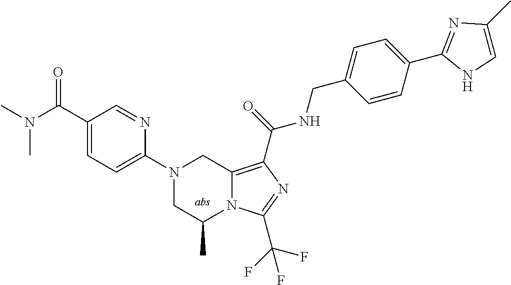 | (S)-7-(5- (dimethylcarbamoyl) pyridin-2-yl)-5-methyl-N- (4-(4-methyl-1H- imidazol-2-yl)benzyl)-3- (trifluoromethyl)-5,6,7,8- tetrahydroimidazo[1,5-a] pyrazine-1-carboxamide | 567.3 |
| E349A | Example 126 | 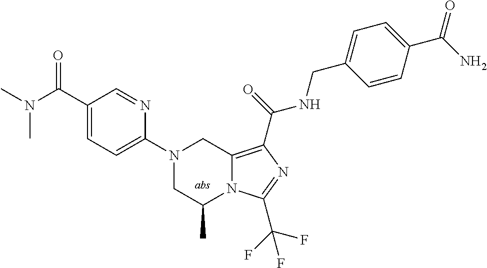 [SMILES: C[C@H]1CN(c2ccc(C(=O)N(C)C)cn2)Cc2c(C(=O)NCc3ccc(C(N)=O)cc3)nc(C(F)(F)F)n21] | (S)-N-(4- carbamoylbenzyl)-7-(5- (dimethylcarbamoyl) pyridin-2-yl)-5-methyl-3- (trifluoromethyl)-5,6,7,8- tetrahydroimidazo[1,5-a] pyrazine-1-carboxamide | 530.3 |
| E350A | Example 126 | 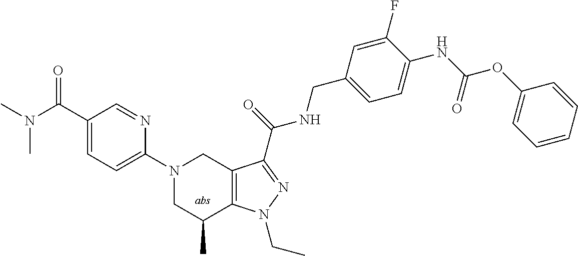 [SMILES: CCn1nc(C(=O)NCc2ccc(NC(=O)Oc3ccccc3)c(F)c2)c2c1[C@@H](C)CN(c1ccc(C(=O)N(C)C)cn1)C2] | phenyl (S)-(4-((5-(5- (dimethylcarbamoyl) pyridin-2-yl)-1-ethyl-7- methyl-4,5,6,7-tetrahydro-1H- pyrazolo[4,3-c]pyridine- 3-carboxamido)methyl)-2- fluorophenyl)carbamate | 600.4 |
| E351A | Example 126 | 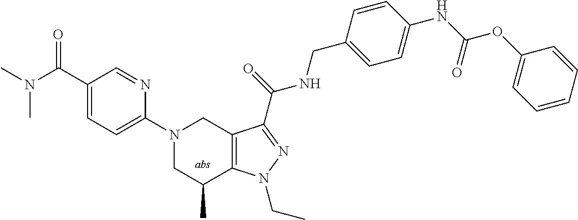 [SMILES: CCn1nc(C(=O)NCc2ccc(NC(=O)Oc3ccccc3)cc2)c2c1[C@@H](C)CN(c1ccc(C(=O)N(C)C)cn1)C2] | phenyl (S)-(4-((5-(5- (dimethylcarbamoyl) pyridin-2-yl)-1-ethyl-7- methyl-4,5,6,7-tetrahydro-1H- pyrazolo[4,3-c]pyridine- 3-carboxamido)methyl) phenyl)carbamate | 582.3 |
| E352A | Example 126 | 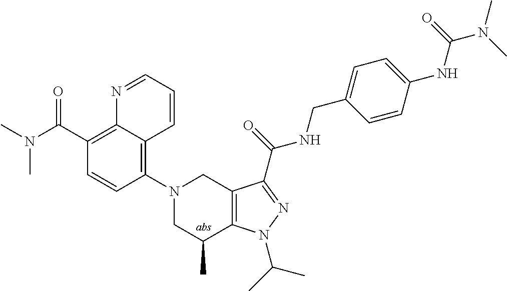 | (S)-5-(3-((4-(3,3- dimethylureido)benzyl) carbamoyl)-1-isopropyl- 7-methyl-1,4,6,7- tetrahydro-5H- pyrazolo[4,3-c]pyridin-5- yl)-N,N-dimethylquinoline- 8-carboxamide | 597.5 |
| E353A | Example 126 | 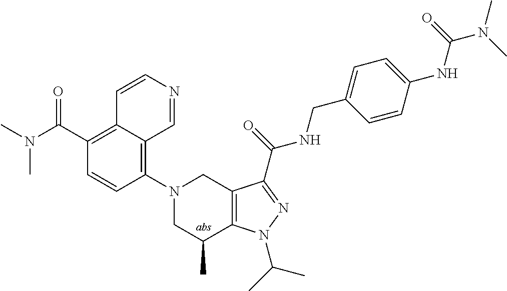 | (S)-8-(3-((4-(3,3- dimethylureido)benzyl) carbamoyl)-1-isopropyl- 7-methyl-1,4,6,7- tetrahydro-5H- pyrazolo[4,3-c]pyridin- 5-yl)-N,N- dimethylisoquinoline-5- carboxamide | 597.5 |
| E354A | Example 126 | 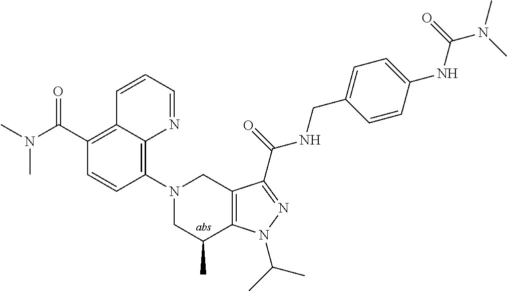 | (S)-8-(3-((4-(3,3- dimethylureido)benzyl) carbamoyl)-1-isopropyl- 7-methyl-1,4,6,7- tetrahydro-5H- pyrazolo[4,3-c]pyridin- 5-yl)-N,N- dimethylquinoline-5- carboxamide | 597.4 |
| E355A | Example 126 | 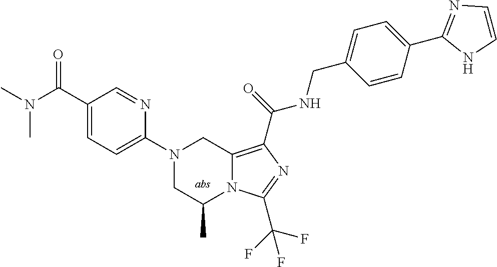 [SMILES: C[C@H]1CN(c2ccc(C(=O)N(C)C)cn2)Cc2c(C(=O)NCc3ccc(-c4ncc[nH]4)cc3)nc(C(F)(F)F)n21] | (S)-N-(4-(1H-imidazol- 2-yl)benzyl)-7-(5- (dimethylcarbamoyl) pyridin-2-yl)-5-methyl-3- 5,6,7,8- tetrahydroimidazo[1,5- a]pyrazine-1-carboxamide | 553.3 |
| E356A | Example 567 | 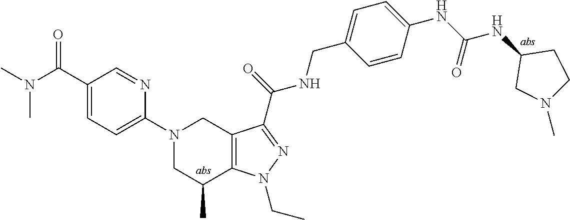 [SMILES: CCn1nc(C(=O)NCc2ccc(NC(=O)N[C@H]3CCN(C)C3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-((S)-1- methylpyrrolidin-3- yl)ureido)benzyl)- 4,5,6,7-tetrahydro-1H- pyrazolo[4,3-c]pyridine- 3-carboxamide | 588.5 |
| E357A | Example 567 | 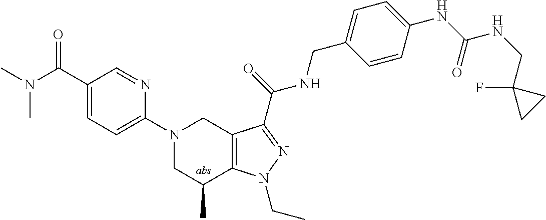 [SMILES: CCn1nc(C(=O)NCc2ccc(NC(=O)NCC3(F)CC3)cc2)c2c1[C@@H](C)CN(c1ccc(C(=O)N(C)C)cn1)C2] | (S)-5-(5- (dimethylcarbamoyl) pyridin-2-yl)-1-ethyl-N-(4- (3-((1- fluorocyclopropyl)methyl) ureido)benzyl)-7- methyl-4,5,6,7-tetrahydro-1H- pyrazolo[4,3-c]pyridine- 3-carboxamide | 577.5 |
| E358A | Example 567 | 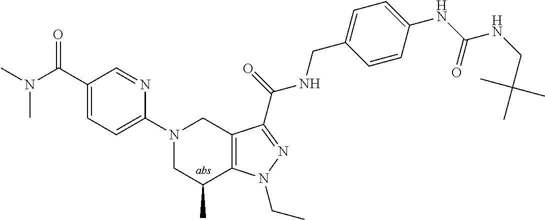 [SMILES: CCn1nc(C(=O)NCc2ccc(NC(=O)NCC(C)(C)C)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3- neopentylureido)benzyl)- 4,5,6,7-tetrahydro-1H- pyrazolo[4,3-c]pyridine- 3-carboxamide | 575.5 |
| E359A | Example 567 | 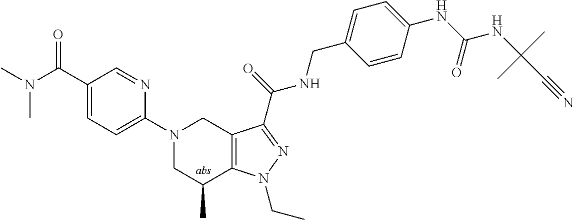 [SMILES: CCn1nc(C(=O)NCc2ccc(NC(=O)NC(C)(C)C#N)cc2)c2c1[C@@H](C)CN(c1ccc(C(=O)N(C)C)cn1)C2] | (S)-N-(4-(3-(2- cyanopropan-2- yl)ureido)benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 572.5 |
| E360A | Example 567 | 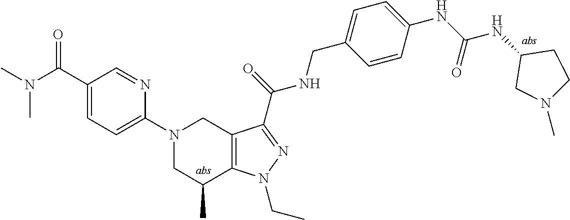 [SMILES: CCn1nc(C(=O)NCc2ccc(NC(=O)N[C@@H]3CCN(C)C3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-((R)-1- methylpyrrolidin-3- yl)ureido)benzyl)- 4,5,6,7-tetrahydro-1H- pyrazolo[4,3-c]pyridine- 3-carboxamide | 588.5 |
| E361A | Example 567 | 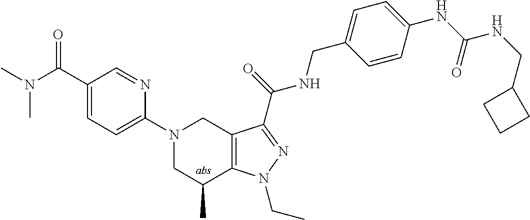 [SMILES: CCn1nc(C(=O)NCc2ccc(NC(=O)NCC3CCC3)cc2)c2c1[C@@H](C)CN(c1ccc(C(=O)N(C)C)cn1)C2] | (S)-N-(4-(3- (cyclobutylmethyl)ureido) benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 573.5 |
| E362A | Example 567 | 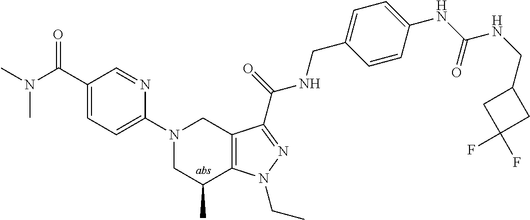 [SMILES: CCn1nc(C(=O)NCc2ccc(NC(=O)NCC3CC(F)(F)C3)cc2)c2c1[C@@H](C)CN(c1ccc(C(=O)N(C)C)cn1)C2] | (S)-N-(4-(3-((3,3- difluorocyclobutyl)methyl) ureido)-3-fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 627.3 |
| E363A | Example 567 | 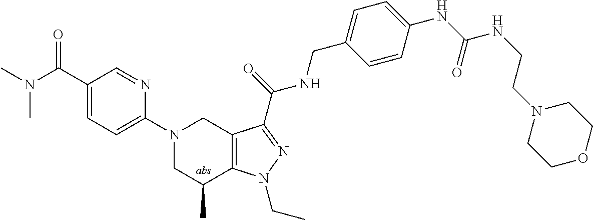 [SMILES: CCn1nc(C(=O)NCc2ccc(NC(=O)NCCN3CCOCC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(2- morpholinoethyl)ureido) benzyl)-4,5,6,7-tetrahydro-1H- pyrazolo[4,3-c]pyridine- 3-carboxamide | 618.6 |
| E364A | Example 126^{b} | 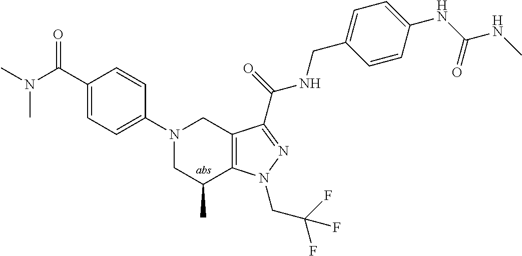 | (S)-5-(4- (dimethylcarbamoyl) phenyl)-7-methyl-N-(4-(3- methylureido)benzyl)-1- (2,2,2-trifluoroethyl)- 4,5,6,7-tetrahydro-1H- pyrazolo[4,3-c]pyridine- 3-carboxamide | 572.3 |
| E365A | Example 126 | 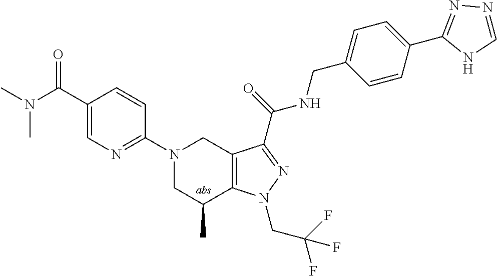 [SMILES: C[C@H]1CN(c2ccc(C(=O)N(C)C)cn2)Cc2c(C(=O)NCc3ccc(-c4nnc[nH]4)cc3)nn(CC(F)(F)F)c21] | (S)-N-(4-(4H-1,2,4- triazol-3-yl)benzyl)-5- (5- (dimethylcarbamoyl) pyridin-2-yl)-7-methyl-1- (2,2,2-trifluoroethyl)- 4,5,6,7-tetrahydro-1H- pyrazolo[4,3-c]pyridine- 3-carboxamide | 568.3 |
| E366A | Example 567 | 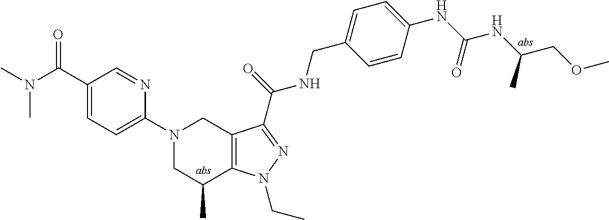 | (S)-5-(5- (dimethylcarbamoyl) pyridin-2-yl)-1-ethyl-N-(4- (3-((R)-1- methoxypropan-2- yl)ureido)benzyl)-7- methyl-4,5,6,7-tetrahydro-1H- pyrazolo[4,3-c]pyridine- 3-carboxamide | 577.5 |
| E367A | Example 567 | 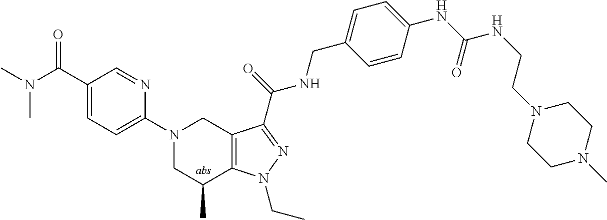 | (S)-5-(5- (dimethylcarbamoyl) pyridin-2-yl)-1-ethyl-7- methyl-N-(4-(3-(2-(4- methylpiperazin-1- yl)ethyl)ureido)benzyl)- 4,5,6,7-tetrahydro-1H- pyrazolo[4,3-c]pyridine- 3-carboxamide | 631.6 |
| E368A | Example 567 | 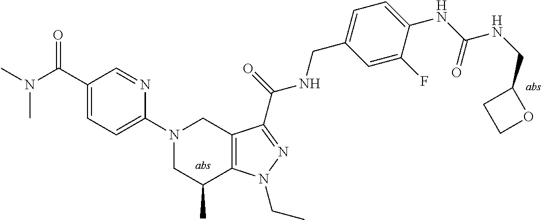 [SMILES: CCn1nc(C(=O)NCc2ccc(NC(=O)NC[C@@H]3CCO3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(((S)- oxetan-2- yl)methyl)ureido)benzyl)-7- methyl-4,5,6,7-tetrahydro-1H- pyrazolo[4,3-c]pyridine- 3-carboxamide | 593.5 |
| E369A | Example 567 | 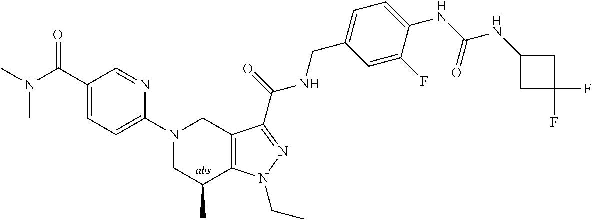 [SMILES: CCn1nc(C(=O)NCc2ccc(NC(=O)NC3CC(F)(F)C3)c(F)c2)c2c1[C@@H](C)CN(c1ccc(C(=O)N(C)C)cn1)C2] | (S)-N-(4-(3-(3,3- difluorocyclobutyl)ureido)- 3-fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 613.5 |
| E370A | Example 567 | 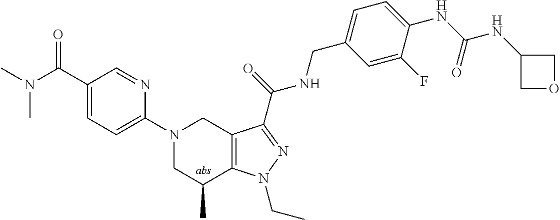 [SMILES: CCn1nc(C(=O)NCc2ccc(NC(=O)NC3COC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(oxetan-3- yl)ureido)benzyl)-7- methyl-4,5,6,7-tetrahydro-1H- pyrazolo[4,3-c]pyridine- 3-carboxamide | 579.5 |
| E371A | Example 567 | 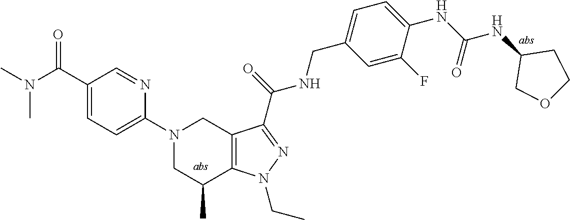 [SMILES: CCn1nc(C(=O)NCc2ccc(NC(=O)N[C@H]3CCOC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((S)- tetrahydrofuran-3- yl)ureido)benzyl)-7- methyl-4,5,6,7-tetrahydro-1H- pyrazolo[4,3-c]pyridine- 3-carboxamide | 593.6 |
| E372A | Example 567 | 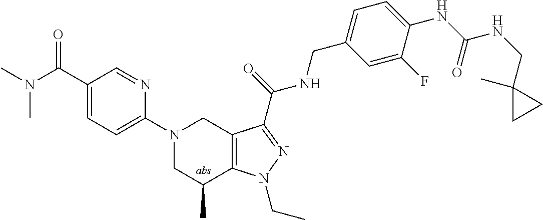 [SMILES: CCn1nc(C(=O)NCc2ccc(NC(=O)NCC3(C)CC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((1- methylcyclopropyl)methyl) ureido)benzyl)-7- methyl-4,5,6,7-tetrahydro-1H- pyrazolo[4,3-c]pyridine- 3-carboxamide | 591.6 |
| E373A | Example 567 | 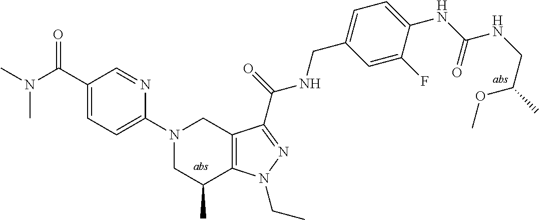 [SMILES: CCn1nc(C(=O)NCc2ccc(NC(=O)NC[C@H](C)OC)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((S)-2- methoxypropyl)ureido) benzyl)-7-methyl- 4,5,6,7-tetrahydro-1H- pyrazolo[4,3-c]pyridine- 3-carboxamide | 595.6 |
| E374A | Example 567 | 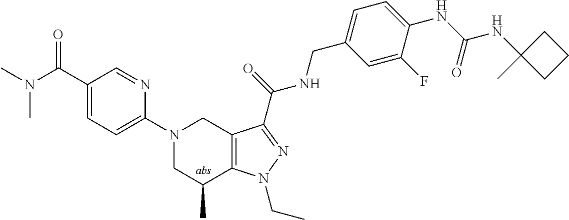 [SMILES: CCn1nc(C(=O)NCc2ccc(NC(=O)NC3(C)CCC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(1- methylcyclobutyl)ureido) benzyl)-7-methyl- 4,5,6,7-tetrahydro-1H- pyrazolo[4,3-c]pyridine- 3-carboxamide | 591.6 |
| E375A | Example 567 | 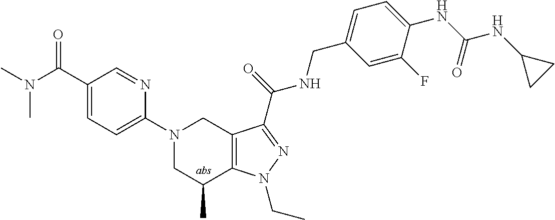 [SMILES: CCn1nc(C(=O)NCc2ccc(NC(=O)NC3CC3)c(F)c2)c2c1[C@@H](C)CN(c1ccc(C(=O)N(C)C)cn1)C2] | (S)-N-(4-(3- cyclopropylureido)-3- fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 563.5 |
| E376A | Example 567 | 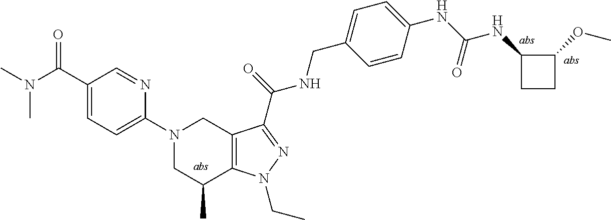 | (S)-5-(5- (dimethylcarbamoyl) pyridin-2-yl)-1-ethyl-N-(4- (3-((1R,2R)-2- methoxycyclobutyl)ureido) benzyl)-7-methyl- 4,5,6,7-tetrahydro-1H- pyrazolo[4,3-c]pyridine- 3-carboxamide | 589.5 |
| E377A | Example 567 | 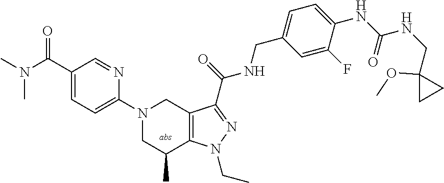 [SMILES: CCn1nc(C(=O)NCc2ccc(NC(=O)NCC3(OC)CC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((1- methoxycyclopropyl) methyl)ureido)benzyl)-7- methyl-4,5,6,7-tetrahydro-1H- pyrazolo[4,3-c]pyridine- 3-carboxamide | 607.6 |
| E378A | Example 567 | 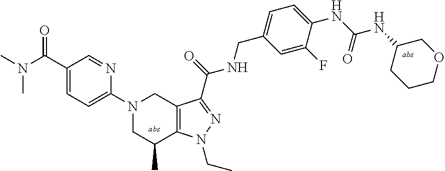 | (S)-5-(5- (dimethylcarbamoyl) pyridin-2-yl)-1-ethyl-N-(3- fluoro-4-(3-((S)- tetrahydro-2H-pyran-3- yl)ureido)benzyl)-7-methyl- 4,5,6,7-tetrahydro-1H- pyrazolo[4,3-c]pyridine- 3-carboxamide | 607.6 |
| E379A | Example 567 | 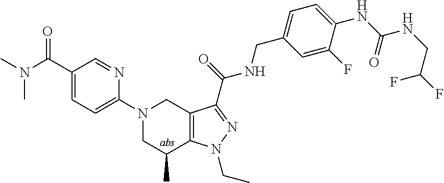 [SMILES: CCn1nc(C(=O)NCc2ccc(NC(=O)NCC(F)F)c(F)c2)c2c1[C@@H](C)CN(c1ccc(C(=O)N(C)C)cn1)C2] | (S)-N-(4-(3-(2,2- difluoroethyl)ureido)-3- fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 587.5 |
| E380A | Example 567 | 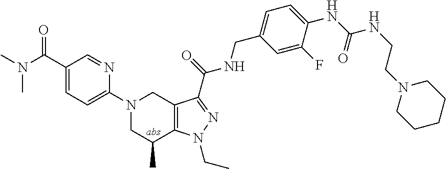 [SMILES: CCn1nc(C(=O)NCc2ccc(NC(=O)NCCN3CCCCC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(2- (piperidin-1-yl)ethyl)ureido) benzyl)-7-methyl- 4,5,6,7-tetrahydro-1H- pyrazolo[4,3-c]pyridine- 3-carboxamide | 634.6 |
| E381A | Example 567 | 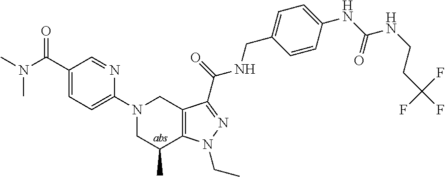 [SMILES: CCn1nc(C(=O)NCc2ccc(NC(=O)NCCC(F)(F)F)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(3,3,3- trifluoropropyl)ureido) benzyl)-4,5,6,7-tetrahydro-1H- pyrazolo[4,3-c]pyridine- 3-carboxamide | 601.5 |
| E382A | Example 567 | 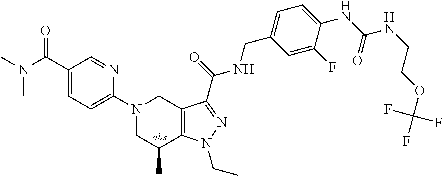 [SMILES: CCn1nc(C(=O)NCc2ccc(NC(=O)NCCOC(F)(F)F)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(2- (trifluoromethoxy)ethyl) ureido)benzyl)-7- methyl-4,5,6,7-tetrahydro-1H- pyrazolo[4,3-c]pyridine- 3-carboxamide | 635.5 |
| E383A | Example 567 | 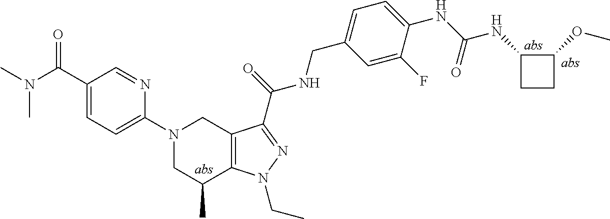 | (S)-5-(5- (dimethylcarbamoyl) pyridin-2-yl)-1-ethyl-N-(3- fluoro-4-(3-((1S,2R)-2- methoxycyclobutyl) ureido)benzyl)-7-methyl- 4,5,6,7-tetrahydro-1H- pyrazolo[4,3-c]pyridine- 3-carboxamide | 607.6 |
| E38A4 | Example 567 | 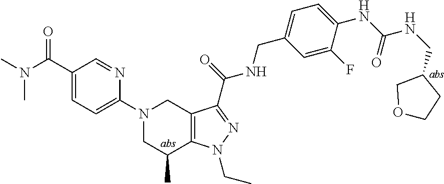 [SMILES: CCn1nc(C(=O)NCc2ccc(NC(=O)NC[C@@H]3CCOC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(((S)- tetrahydrofuran-3- yl)methyl)ureido)benzyl)- 7-methyl-4,5,6,7- tetrahydro-1H- pyrazolo[4,3-c]pyridine- 3-carboxamide | 607.6 |
| E385A | Example 567 | 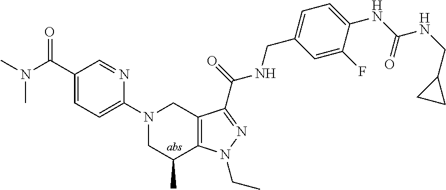 [SMILES: CCn1nc(C(=O)NCc2ccc(NC(=O)NCC3CC3)c(F)c2)c2c1[C@@H](C)CN(c1ccc(C(=O)N(C)C)cn1)C2] | (S)-N-(4-(3- (cyclopropylmethyl)ureido)- 3-fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 577.5 |
| E386A | Example 567 | 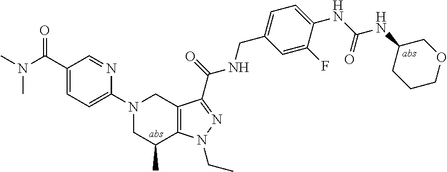 [SMILES: CCn1nc(C(=O)NCc2ccc(NC(=O)N[C@@H]3CCCOC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((R)- tetrahydro-2H-pyran-3- yl)ureido)benzyl)-7- methyl-4,5,6,7-tetrahydro-1H- pyrazolo[4,3-c]pyridine- 3-carboxamide | 607.6 |
| E387A | Example 567 | 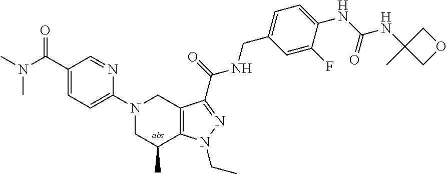 [SMILES: CCn1nc(C(=O)NCc2ccc(NC(=O)NC3(C)COC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(3- methyloxetan-3- yl)ureido)benzyl)-7- methyl-4,5,6,7-tetrahydro-1H- pyrazolo[4,3-c]pyridine- 3-carboxamide | 593.5 |
| E388A | Example 567 | 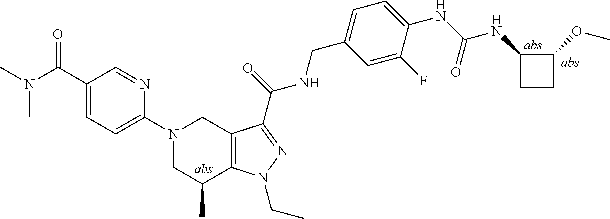 | (S)-5-(5- (dimethylcarbamoyl)pyridin- 2-yl)-1-ethyl-N-(3- fluoro-4-(3-((1R,2R)-2- methoxycyclobutyl)ureido) benzyl)-7-methyl- 4,5,6,7-tetrahydro-1H- pyrazolo[4,3-c]pyridine- 3-carboxamide | 607.6 |
| E389A | Example 567 | 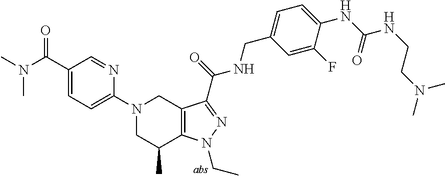 | (S)-N-(4-(3-(2- (dimethylamino)ethyl) ureido)-3-fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 594.5 |
| E390A | Example 567 | 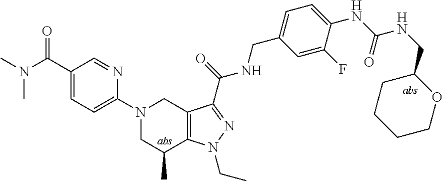 [SMILES: CCn1nc(C(=O)NCc2ccc(NC(=O)NC[C@@H]3CCCCO3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-(((S)- tetrahydro-2H-pyran-2- yl)methyl)ureido)benzyl)-7- methyl-4,5,6,7-tetrahydro-1H- pyrazolo[4,3-c]pyridine- 3-carboxamide | 621.6 |
| E391A | Example 567 | 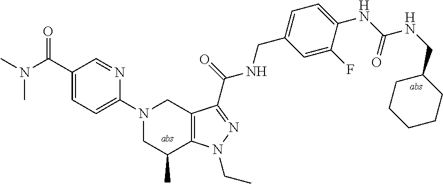 | (S)-5-(5- (dimethylcarbamoyl)pyridin- 2-yl)-1-ethyl-N-(3- fluoro-4-(3-((1S,2S)-2- methoxycyclobutyl)ureido) benzyl)-7-methyl- 4,5,6,7-tetrahydro-1H- pyrazolo[4,3-c]pyridine- 3-carboxamide | 607.6 |
| E392A | Example 567 | 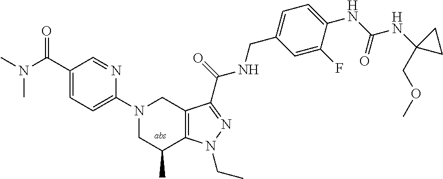 [SMILES: CCn1nc(C(=O)NCc2ccc(NC(=O)NC3(COC)CC3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(4- (3-(1- (methoxymethyl)cyclopropyl) ureido)benzyl)-7-methyl- 4,5,6,7-tetrahydro-1H- pyrazolo[4,3-c]pyridine- 3-carboxamide | 589.5 |
| E393A | Example 567 | 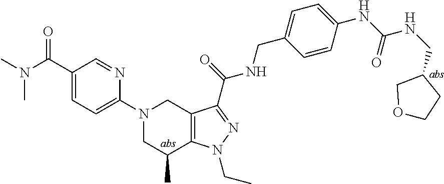 [SMILES: CCn1nc(C(=O)NCc2ccc(NC(=O)NC[C@@H]3CCOC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((S)- tetrahydrofuran-3- yl)methyl)ureido)benzyl)- 4,5,6,7-tetrahydro-1H- pyrazolo[4,3-c]pyridine- 3-carboxamide | 589.6 |
| E394A | Example 567 | 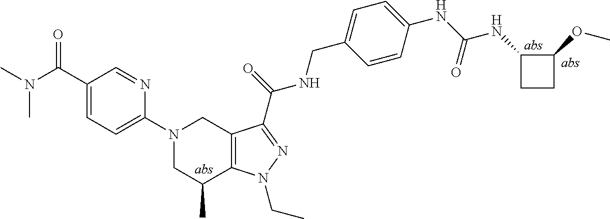 | (S)-5-(5- (dimethylcarbamoyl) pyridin-2-yl)-1-ethyl-N-(4- (3-((1S,2S)-2- methoxycyclobutyl) ureido)benzyl)-7-methyl- 4,5,6,7-tetrahydro-1H- pyrazolo[4,3-c]pyridine- 3-carboxamide | 589.5 |
| E395A | Example 567 | 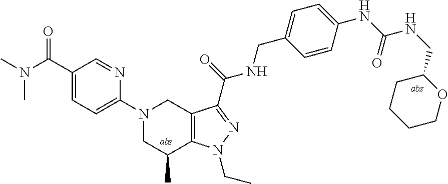 [SMILES: CCn1nc(C(=O)NCc2ccc(NC(=O)NC[C@H]3CCCCO3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((R)- tetrahydro-2H-pyran-2- yl)methyl)ureido)benzyl)- 4,5,6,7-tetrahydro-1H- pyrazolo[4,3-c]pyridine- 3-carboxamide | 603.5 |
| E396A | Example 567 | 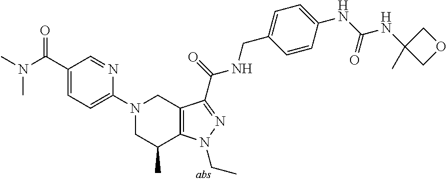 [SMILES: CCn1nc(C(=O)NCc2ccc(NC(=O)NC3(C)COC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(3- methyloxetan-3- yl)ureido)benzyl)- 4,5,6,7-tetrahydro-1H- pyrazolo[4,3-c]pyridine- 3-carboxamide | 575.5 |
| E397A | Example 567 | 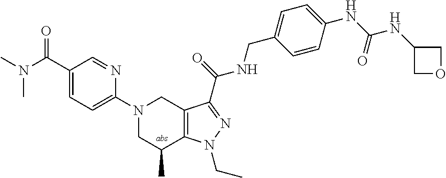 [SMILES: CCn1nc(C(=O)NCc2ccc(NC(=O)NC3COC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(oxetan- 3-yl)ureido)benzyl)- 4,5,6,7-tetrahydro-1H- pyrazolo[4,3-c]pyridine- 3-carboxamide | 561.5 |
| E398A | Example 567 | 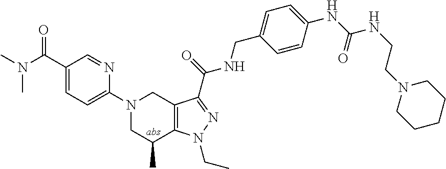 [SMILES: CCn1nc(C(=O)NCc2ccc(NC(=O)NCCN3CCCCC3)cc2)c2c1[C@@H](C)CN(c1ccc(C(=O)N(C)C)cn1)C2] | (S)-5-(5- (dimethylcarbamoyl)pyridin- 2-yl)-1-ethyl-7-methyl-N-(4- (3-(2-(piperidin-1- yl)ethyl)ureido)benzyl)- 4,5,6,7-tetrahydro-1H- pyrazolo[4,3-c]pyridine- 3-carboxamide | 616.6 |
| E399A | Example 567 | 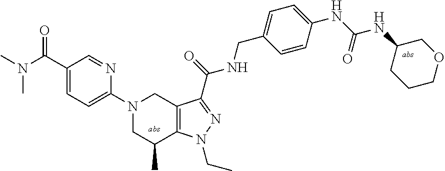 [SMILES: CCn1nc(C(=O)NCc2ccc(NC(=O)N[C@@H]3CCCOC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-((R)- tetrahydro-2H-pyran-3- yl)ureido)benzyl)- 4,5,6,7-tetrahydro-1H- pyrazolo[4,3-c]pyridine- 3-carboxamide | 589.5 |
| E400A | Example 567 | 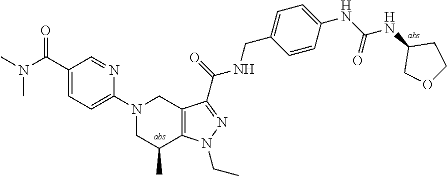 [SMILES: CCn1nc(C(=O)NCc2ccc(NC(=O)N[C@H]3CCOC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-((S)- tetrahydrofuran-3- yl)ureido)benzyl)- 4,5,6,7-tetrahydro-1H- pyrazolo[4,3-c]pyridine- 3-carboxamide | 575.5 |
| E401A | Example 567 | 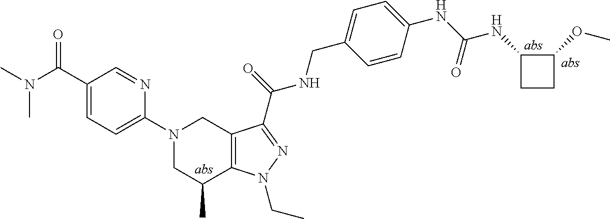 | (S)-5-(5- (dimethylcarbamoyl) pyridin-2-yl)-1-ethyl-N-(4- (3-((1S,2R)-2- methoxycyclobutyl) ureido)benzyl)-7-methyl- 4,5,6,7-tetrahydro-1H- pyrazolo[4,3-c]pyridine- 3-carboxamide | 589.5 |
| E402A | Example 567 | 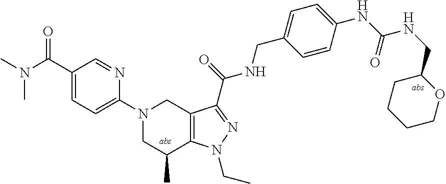 [SMILES: CCn1nc(C(=O)NCc2ccc(NC(=O)NC[C@@H]3CCCCO3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((S)- tetrahydro-2H-pyran-2- yl)methyl)ureido)benzyl)- 4,5,6,7-tetrahydro-1H- pyrazolo[4,3-c]pyridine- 3-carboxamide | 603.5 |
| E403A | Example 567 | 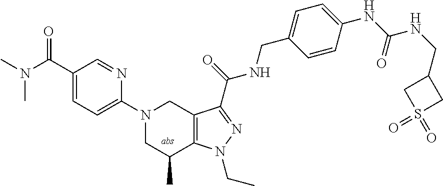 [SMILES: CCn1nc(C(=O)NCc2ccc(NC(=O)NCC3CS(=O)(=O)C3)cc2)c2c1[C@@H](C)CN(c1ccc(C(=O)N(C)C)cn1)C2] | (S)-5-(5- (dimethylcarbamoyl) pyridin-2-yl)-N-(4-(3-((1,1- dioxidothietan-3- yl)methyl)ureido)benzyl)- 1-ethyl-7-methyl- 4,5,6,7-tetrahydro-1H- pyrazolo[4,3-c]pyridine- 3-carboxamide | 623.5 |
| E404A | Example 567 | 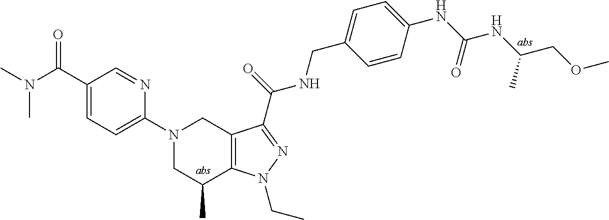 | (S)-5-(5- (dimethylcarbamoyl) pyridin-2-yl)-1-ethyl-N-(4- (3-((S)-1- methoxypropan-2- yl)ureido)benzyl)-7- methyl-4,5,6,7-tetrahydro-1H- pyrazolo[4,3-c]pyridine- 3-carboxamide | 577.5 |
| E405A | Example 567 | 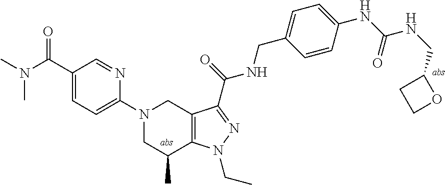 [SMILES: CCn1nc(C(=O)NCc2ccc(NC(=O)NC[C@H]3CCO3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((R)- oxetan-2- yl)methyl)ureido)benzyl)- 4,5,6,7-tetrahydro-1H- pyrazolo[4,3-c]pyridine- 3-carboxamide | 575.5 |
| E406A | Example 567 | 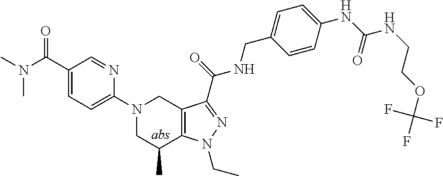 [SMILES: CCn1nc(C(=O)NCc2ccc(NC(=O)NCCOC(F)(F)F)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(2- (trifluoromethoxy)ethyl) ureido)benzyl)- 4,5,6,7-tetrahydro-1H- pyrazolo[4,3-c]pyridine- 3-carboxamide | 617.5 |
| E407A | Example 567 | 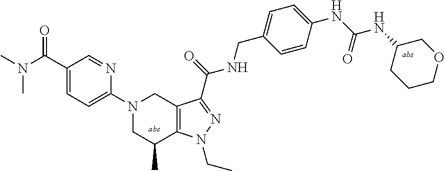 | (S)-5-(5- (dimethylcarbamoyl) pyridin-2-yl)-1-ethyl-7- methyl-N-(4-(3-((S)- tetrahydro-2H-pyran-3- yl)ureido)benzyl)- 4,5,6,7-tetrahydro-1H- pyrazolo[4,3-c]pyridine- 3-carboxamide | 589.5 |
| E408A | Example 567 | 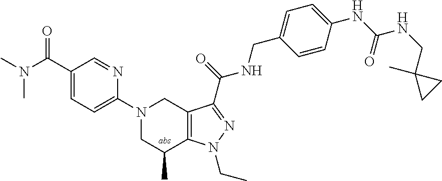 [SMILES: CCn1nc(C(=O)NCc2ccc(NC(=O)NCC3(C)CC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-((1- methylcyclopropyl) methyl)ureido)benzyl)- 4,5,6,7-tetrahydro-1H- pyrazolo[4,3-c]pyridine- 3-carboxamide | 573.5 |
| E409A | Example 567 | 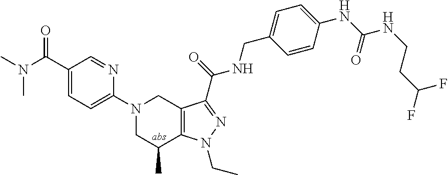 [SMILES: CCn1nc(C(=O)NCc2ccc(NC(=O)NCCC(F)F)cc2)c2c1[C@@H](C)CN(c1ccc(C(=O)N(C)C)cn1)C2] | (S)-N-(4-(3-(3,3- difluoropropyl)ureido) benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 583.5 |
| E410A | Example 567 | 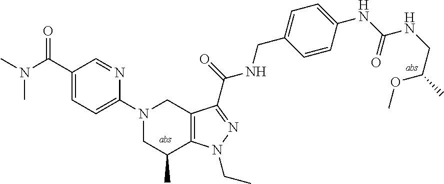 [SMILES: CCn1nc(C(=O)NCc2ccc(NC(=O)NC[C@H](C)OC)cc2)c2c1[C@@H](C)CN(c1ccc(C(=O)N(C)C)cn1)C2] | (S)-5-(5- (dimethylcarbamoyl) pyridin-2-yl)-1-ethyl-N-(4- (3-((S)-2- methoxypropyl)ureido) benzyl)-7-methyl- 4,5,6,7-tetrahydro-1H- pyrazolo[4,3-c]pyridine- 3-carboxamide | 577.5 |
| E411A | Example 567 | 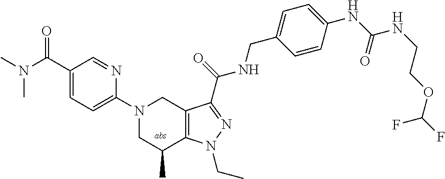 [SMILES: CCn1nc(C(=O)NCc2ccc(NC(=O)NCCOC(F)F)cc2)c2c1[C@@H](C)CN(c1ccc(C(=O)N(C)C)cn1)C2] | (S)-N-(4-(3-(2- (difluoromethoxy)ethyl) ureido)benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 599.5 |
| E412A | Example 567 | 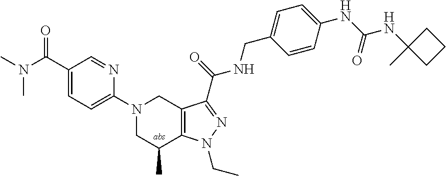 [SMILES: CCn1nc(C(=O)NCc2ccc(NC(=O)NC3(C)CCC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(1- methylcyclobutyl)ureido) benzyl)- 4,5,6,7-tetrahydro-1H- pyrazolo[4,3-c]pyridine- 3-carboxamide | 573.5 |
| E413A | Example 567 | 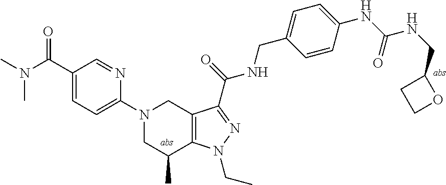 [SMILES: CCn1nc(C(=O)NCc2ccc(NC(=O)NC[C@@H]3CCO3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((S)- oxetan-2- yl)methyl)ureido)benzyl)- 4,5,6,7-tetrahydro-1H- pyrazolo[4,3-c]pyridine- 3-carboxamide | 575.5 |
| E414A | Example 567 | 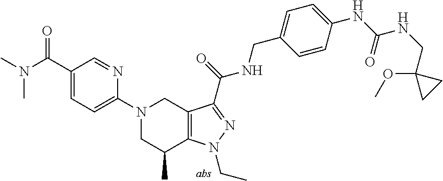 [SMILES: CCn1nc(C(=O)NCc2ccc(NC(=O)NCC3(OC)CC3)cc2)c2c1[C@@H](C)CN(c1ccc(C(=O)N(C)C)cn1)C2] | (S)-5-(5- (dimethylcarbamoyl) pyridin-2-yl)-1-ethyl-N-(4- (3-((1- methoxycyclopropyl) methyl)ureido)benzyl)-7- methyl-4,5,6,7-tetrahydro-1H- pyrazolo[4,3-c]pyridine- 3-carboxamide | 589.5 |
| E415A | Example 567 | 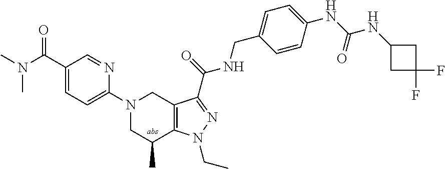 [SMILES: CCn1nc(C(=O)NCc2ccc(NC(=O)NC3CC(F)(F)C3)cc2)c2c1[C@@H](C)CN(c1ccc(C(=O)N(C)C)cn1)C2] | (S)-N-(4-(3-(3,3- difluorocyclobutyl)ureido) benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 595.5 |
| E416A | Example 567 | 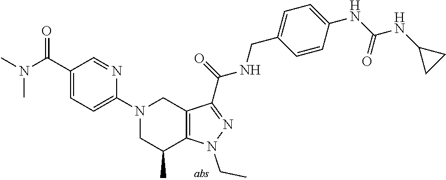 [SMILES: CCn1nc(C(=O)NCc2ccc(NC(=O)NC3CC3)cc2)c2c1[C@@H](C)CN(c1ccc(C(=O)N(C)C)cn1)C2] | (S)-N-(4-(3- cyclopropylureido) benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 545.5 |
| E417A | Example 567 | 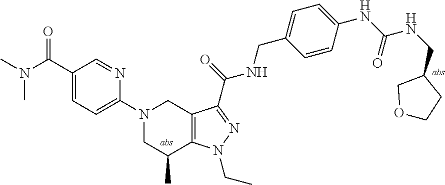 [SMILES: CCn1nc(C(=O)NCc2ccc(NC(=O)NC[C@H]3CCOC3)cc2)c2c1[C@@H](C)CN(c1ccc(C(=O)N(C)C)cn1)C2] | (S)-5-(5- (dimethylcarbamoyl) pyridin-2-yl)-1-ethyl-7- methyl-N-(4-(3-(((R)- tetrahydrofuran-3- yl)methyl)ureido)benzyl)- 4,5,6,7-tetrahydro-1H- pyrazolo[4,3-c]pyridine- 3-carboxamide | 589.5 |
| E418A | Example 567 | 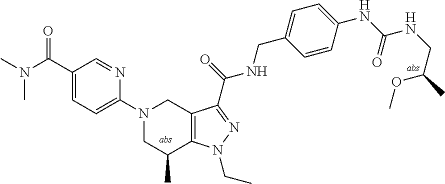 [SMILES: CCn1nc(C(=O)NCc2ccc(NC(=O)NC[C@@H](C)OC)cc2)c2c1[C@@H](C)CN(c1ccc(C(=O)N(C)C)cn1)C2] | (S)-5-(5- (dimethylcarbamoyl) pyridin-2-yl)-1-ethyl-N-(4- (3-((R)-2- methoxypropyl)ureido) benzyl)-7-methyl- 4,5,6,7-tetrahydro-1H- pyrazolo[4,3-c]pyridine- 3-carboxamide | 577.5 |
| E419A | Example 567 | 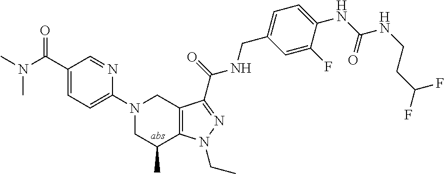 [SMILES: CCn1nc(C(=O)NCc2ccc(NC(=O)NCCC(F)F)c(F)c2)c2c1[C@@H](C)CN(c1ccc(C(=O)N(C)C)cn1)C2] | (S)-N-(4-(3-(3,3- difluoropropyl)ureido)- 3-fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 601.5 |
| E420A | Example 567 | 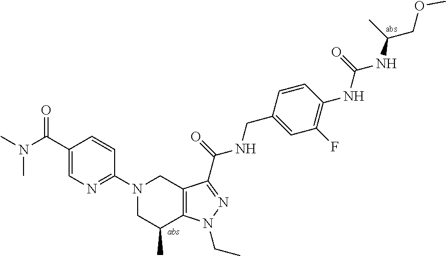 | (S)-5-(5- (dimethylcarbamoyl) pyridin-2-yl)-1-ethyl-N-(3- fluoro-4-(3-((S)-1- methoxypropan-2-yl) ureido)benzyl)-7- methyl-4,5,6,7-tetrahydro-1H- pyrazolo[4,3-c]pyridine- 3-carboxamide | 595.6 |
| E421A | Example 567 | 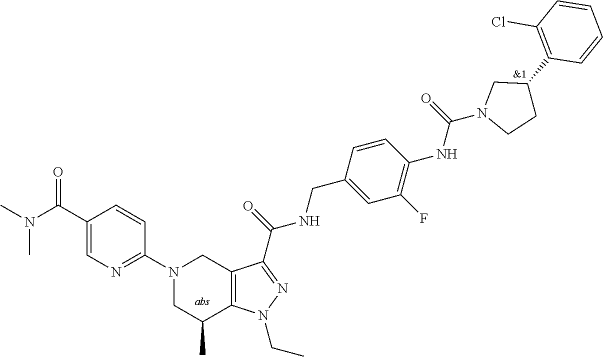 [SMILES: CCn1nc(C(=O)NCc2ccc(NC(=O)N3CC[C@@H](c4ccccc4Cl)C3)c(F)c2)c2c1[C@@H](C)CN(c1ccc(C(=O)N(C)C)cn1)C2] | (S)-N-(4-((RS)-3-(2- chlorophenyl)pyrrolidine-1- carboxamido)-3- fluorobenzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 688.6 |
| E422A | Example 567 | 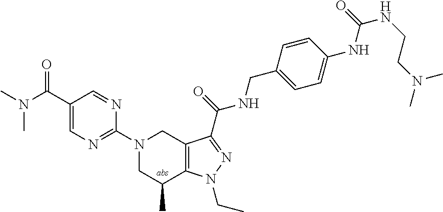 | (S)-N-(4-(3-(2- (dimethylamino)ethyl) ureido)benzyl)-5-(5- (dimethylcarbamoyl) pyridin-2-yl)-1-ethyl-7- methyl-4,5,6,7-tetrahydro-1H- pyrazolo[4,3-c]pyridine- 3-carboxamide | 576.5 |
| E423A | Example 567 | 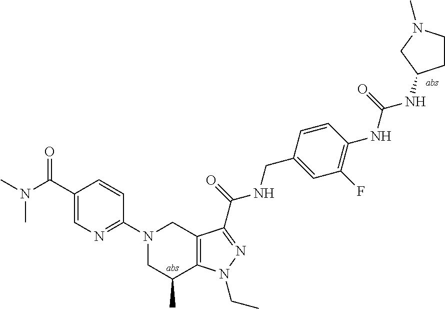 [SMILES: CCn1nc(C(=O)NCc2ccc(NC(=O)N[C@H]3CCN(C)C3)c(F)c2)c2c1[C@@H](C)CN(c1ccc(C(=O)N(C)C)cn1)C2] | (S)-5-(5- (dimethylcarbamoyl) pyridin-2-yl)-1-ethyl-N-(3- fluoro-4-(3-((S)-1- methylpyrrolidin-3- yl)ureido)benzyl)-7- methyl-4,5,6,7-tetrahydro-1H- pyrazolo[4,3-c]pyridine- 3-carboxamide | 606.4 |
| E424A | Example 395 | 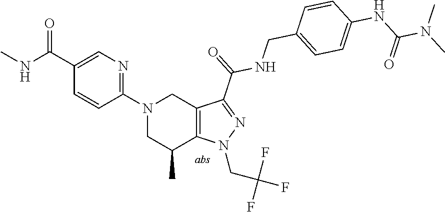 | (S)-N-(4-(3,3- dimethylureido)benzyl)- 7-methyl-5-(5- (methylcarbamoyl) pyridin-2-yl)-1-(2,2,2- trifluoroethyl)- 4,5,6,7-tetrahydro-1H- pyrazolo[4,3-c]pyridine- 3-carboxamide | 573.3 |
| E425A | Example 127 | 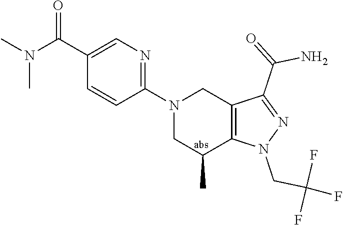 [SMILES: C[C@H]1CN(c2ccc(C(=O)N(C)C)cn2)Cc2c(C(N)=O)nn(CC(F)(F)F)c21] | (S)-5-(5- (dimethylcarbamoyl) pyridin-2-yl)-7-methyl-1- (2,2,2-trifluoroethyl)- 4,5,6,7-tetrahydro-1H- pyrazolo[4,3-c]pyridine- 3-carboxamide | 411.3 |
| E426A | Example 498 | 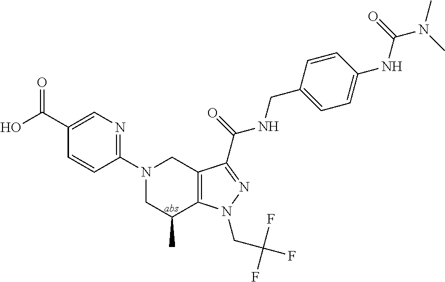 [SMILES: C[C@H]1CN(c2ccc(C(=O)O)cn2)Cc2c(C(=O)NCc3ccc(NC(=O)N(C)C)cc3)nn(CC(F)(F)F)c21] | (S)-6-(3-((4-(3,3- dimethylureido)benzyl) carbamoyl)-7-methyl-1- (2,2,2-trifluoroethyl)- 1,4,6,7-tetrahydro-5H- pyrazolo[4,3-c]pyridin- 5-yl)nicotinic acid | 560.3 |

Example # * = chiral purification data listed below
Example 127^{a}: additional Suzuki cross-coupling step; A solution of (S)-5-(5-(3-bromo-6,7-dihydro-5H-pyrrolo[3,4-b]pyridine-6-carbonyl)pyridin-2-yl)-N-(4-(3,3-dimethylureido)benzyl)-1-isopropyl-7-methyl-
4,5,6,7-tetrahydro-1H-pyrazolo[4,3-c]pyridine-3-carboxamide (1.0 equivalent), potassium dimethylaminomethyltrifluoroboronate (1.0 equivalent) and Na₂CO₃ (2.5 equivalents) in (4:1, dioxane: H₂O) was degassed
with nitrogen gas (3×) then cataCXium ® A Pd G3 (0.2 equivalent) was added. The reaction mixture was degassed with nitrogen gas (3×) then stirred at 110 °C for 2 h. The suspension was poured into H₂O then
filtered. The filter cake was washed with H₂O (5 mL × 3) then collected and dissolved in DMF. The solution was purified by reverse phase HPLC (WePure Biotech XP tC₁₈ 150 mm × 40 mm × 7 μm; Mobile phase A:
H₂O (0.225% formic acid)/Mobile Phase B: ACN; gradient: 9-49% of Mobile Phase B over 12 min; flow rate: 60 mL/min to provide E156A.
Example 126^{b} or 127^{b}: altered palladium coupling step; To a stirred solution of (S)-7-methyl-1-(2,2,2-trifluoroethyl)-4,5,6,7-tetrahydro-1H-pyrazolo[4,3-c]pyridine-3-carboxylic acid (1 equivalent), 4-bromo-N,N-
dimethylbenzamide (1 equivalent) in DMF (0.2M) was added K₃PO₄ (3 equivalents) and RuPhos Pd G3 (0.1 equivalent). The reaction mixture was degassed with nitrogen gas for 3 min then stirred at 95 °C for 16 h.
The solution was diluted with H₂O (10 mL) and extracted with EtOAc (10 mL × 2). The organic layer was discarded then the aqueous phase was adjusted to pH ~5 with 1M HCl. The acidic aqueous phase was extracted
with EtOAc (15 mL × 3). The combined organic layer was dried over Na₂SO₄ and filtered. The filtrate was concentrated in vacuo to provide (S)-5-(4-(dimethylcarbamoyl)phenyl)-7-methyl-1-(2,2,2-trifluoroethyl)-
4,5,6,7-tetrahydro-1H-pyrazolo[4,3-c]pyridine-3-carboxylic acid.
Example 126^{c} or 127^{c}: altered palladium coupling step; To a reaction mixture of rel-(R)-3-ethyl-5-methyl-5,6,7,8-tetrahydroimidazo[1,5-a]pyrazine-1-carboxylic acid (1.00 equivalent) and 4-bromo-N,N-dimethyl-
benzamide (1.20 equivalents) in tert-amyl alcohol (0.13M) was added KOTMS (3.50 equivalents) and P(t-Bu)₃ Pd G2 (0.07 equivalent) under nitrogen gas. The reaction mixture was stirred at 110 °C for 1.5 h then
concentrated in vacuo to remove half of the tert-amyl alcohol. To the suspension was added H₂O (25 mL) then extracted with EtOAc (25 mL × 2) then 2-methyltetrahydrofuran (25 mL × 2). The combined organic
phase was discarded. The aqueous phase was adjusted to pH = 5-6 with 1M HCl then extracted with (1:10, MeOH: DCM)(25 mL × 10). The combined organic layer was dried with Na₂SO₄ then concentrated in
vacuo to provide rel-(R)-7-(4-(dimethylcarbamoyl)phenyl)-3-ethyl-5-methyl-5,6,7,8-tetrahydroimidazo[1,5-a]pyrazine-1-carboxylic acid.
Example 126^{d} or 127^{d}: additional Buchwald coupling; To a reaction mixture of (S)-N-(4-bromobenzyl)-5-(5-(dimethylcarbamoyl)pyridin-2-yl)-1-ethyl-7-methyl-4,5,6,7-tetrahydro-1H-pyrazolo[4,3-c]pyridine-3-
carboxamide (1.00 equivalent), pyridazin-3-amine (2.00 equivalents), Cs₂CO₃ (2.50 equivalents) and tBuBrettPhos Pd G3 (0.05 equivalent) was added dioxane (1 mL) then the mixture degassed with nitrogen gas for
a few minutes. The reaction mixture was stirred at 90 °C for 3.5 hours then filtered through Celite. The filtrate was concentrated in vacuo then purified by reverse phase HPLC (Sunfire C₁₈ 100 mm × 19 mm × 5 μm column,
Mobile Phase A: H₂O/Mobile Phase B: ACN (0.05% TFA), 5 to 30% of Mobile Phase B over 8.5 min then to 95% of Mobile Phase B over 0.5 min then held at 95% of Mobile Phase B for 1.0 min, flow rate: 25 mL/min)
to provide to provide E310A.
Example 193^{a}: additional deprotection step; A solution of (1:4, TFA: DCM)(0.2 mL) was added to the tert-butyloxycarbonyl protected amine (20 μmol) then shook at 30 °C for 2 h. The reaction mixture was concentrated
in vacuo then purified by reverse phase HPLC to provide the deprotected amine.
Example 193^{b}: altered palladium coupling conditions: A reaction mixture of tert-butyl 4-iodobenzoate (1.3 equivalents), ethyl (S)-1-isopropyl-7-methyl-4,5,6,7-tetrahydro-1H-pyrazolo[4,3-c]pyridine-3-carboxylate
hydrochloride (1.0 equivalent), RuPhos Pd G4 (0.15 equivalent) and K₃PO₄ (3 equivalents) in tert-amyl alcohol (20 mL) sequentially to give a white slurry. The reaction mixture was degassed with nitrogen gas for
3 min then stirred at 60 °C for ~96 h. The reaction mixture has turned into a yellow slurry then was filtered. The filter cake was dissolved in a mixture of H₂O and DCM. The layers were separated and the
aqueous layer was further extracted by DCM (2×). The combined organic layer and filtrate were collected, dried over Na₂SO₄, concentrated in vacuo. The residue was purified by column chromatography (silica gel;
0-40% EtOAc: heptane) to provide ethyl (S)-5-(4-(tert-butoxycarbonyl)phenyl)-1-isopropyl-7-methyl-4,5,6,7-tetrahydro-1H-pyrazolo[4,3-c]pyridine-3-carboxylate.

### Chiral Purification:

Example E254A and E255A: A mixture was separated into its component isomers using SFC purification (DAICEL CHIRALPAK AD, 250 mm×50 mm×10 μm column; Mobile phase A: CO₂; Mobile phase B: isopropyl alcohol (0.1% NH₄OH); isocratic: 55% of Mobile Phase B; flow rate: 200 mL/min; column temperature: 40 °C; backpressure: 100 bar). The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic: 40% of Mobile Phase B; flow rate: 4.0 mL/min; backpressure: 1500 psi; column temperature: 35 °C to provide Example E254A: peak 1 ((SFC-MS) m/z (M+H)⁺=550.6 at retention time: 1.262 min, 100% ee) and Example E255A: peak 2 ((SFC-MS) m/z (M+H)⁺=550.6 at retention time: 3.225 min, 100% ee).

Example E256A and E257A: A mixture was separated into its component isomers using SFC purification (DAICEL CHIRALPAK AD, 250 mm×30 mm×10 μm column; Mobile phase A: CO₂; Mobile phase B: EtOH (0.1% NH₄OH); isocratic: 50% of Mobile Phase B; flow rate: 120 mL/min; column temperature: 40 °C; backpressure: 100 bar). The chiral purity was determined using SFC analytical method on Chiralpak AD-3 50 mm×4.6 mm×3 μm column; Mobile phase A: CO₂; Mobile phase B: EtOH (0.2% NH₃); isocratic: 40% of Mobile Phase B; flow rate: 3.5 mL/min; backpressure: 1500 psi; column temperature: 35 °C to provide Example E256A: peak 1 ((SFC-MS) m/z (M+H)+=568.5 at retention time: 0.989 min, 100% ee) and Example E257A: peak 2 ((SFC-MS) m/z (M+H)+=568.6 at retention time: 2.123 min, 100% ee).

Example E258A and E259A: A mixture was separated into its component isomers using SFC purification (DAICEL CHIRALPAK AD, 250 mm×30 mm×10 μm column; Mobile phase A: CO₂; Mobile phase B: EtOH (0.1% NH₄OH); isocratic: 50% of Mobile Phase B over 10 min; flow rate: 120 mL/min). The chiral purity was determined using SFC analytical method on Chiralpak AD-3 50 mm×4.6 mm×3 μm column; Mobile phase A: CO₂; Mobile phase B: EtOH (0.2% NH₃); isocratic: 40% of Mobile Phase B; flow rate: 4.0 mL/min; backpressure: 1500 psi; column temperature: 35 °C to provide Example E258A: peak 1 ((SFC-MS) m/z (M+H)⁺=572.4 at retention time: 0.700 min, 100% ee) and Example E259A: peak 2 ((SFC-MS) m/z (M+H)⁺=572.4 at retention time: 1.248 min, 100% ee).

Example E260A and E261A: A mixture was separated into its component isomers using SFC purification (DAICEL CHIRALPAK AD, 250 mm×30 mm×10 μm column; Mobile phase A: CO₂; Mobile phase B: (4:1, isopropyl alcohol: ACN)(0.1% NH₄OH); isocratic: 50% of Mobile Phase B; flow rate: 150 mL/min; backpressure: 100 bar; column temperature: 40 °C). The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic: 40% of Mobile Phase B; flow rate: 4.0 mL/min; backpressure: 1500 psi; column temperature: 35 °C to provide Example E260A: peak 1 ((SFC-MS) m/z (M+H)⁺=551.4 at retention time: 0.883 min, 100% ee) and Example E261A: peak 2 ((SFC-MS) m/z (M+H)⁺=551.5 at retention time: 1.856 min, 100% ee). Example E264A and E265A: A mixture was separated into its component isomers using SFC purification (DAICEL CHIRALPAK AD, 250 mm×30 mm×10 μm column; Mobile phase A: CO₂; Mobile phase B: (4:1, EtOH: DCM)(0.1% NH₄OH); isocratic: 50% of Mobile Phase B; flow rate: 120 mL/min; backpressure: 100 bar; column temperature: 40 °C). The chiral purity was determined using SFC analytical method on Chiralpak AD-3 50 mm×4.6 mm×3 μm column; Mobile phase A: CO₂; Mobile phase B: EtOH (0.2% NH₃); isocratic: 40% of Mobile Phase B; flow rate: 4.0 mL/min; backpressure: 1500 psi; column temperature: 35 °C to provide Example E264A: peak 1 ((SFC-MS) m/z (M+H)⁺=569.4 at retention time: 0.672 min, 100% ee) and Example E265A: peak 2 ((SFC-MS) m/z (M+H)⁺=569.4 at retention time: 1.278 min, 99% ee).

Example E266A and E267A: A mixture was separated into its component isomers using SFC purification (DAICEL CHIRALPAK AD, 250 mm×30 mm×10 μm column; Mobile phase A: CO₂; Mobile phase B: (4:1, isopropyl alcohol: ACN)(0.1% NH₄OH); isocratic: 45% of Mobile Phase B over 50 min; flow rate: 150 mL/min). The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic: 40% of Mobile Phase B; flow rate: 4.0 mL/min; backpressure: 1500 psi; column temperature: 35 °C to provide Example E266A: peak 1 ((SFC-MS) m/z (M+H)⁺=591.4 at retention time: 0.434 min, 100% ee) and Example E267A: peak 2 ((SFC-MS) m/z (M+H)⁺=591.4 at retention time: 1.102 min, 100% ee).

Example E268A and E269A: A mixture was separated into its component isomers using SFC purification (DAICEL CHIRALPAK AD, 250 mm×30 mm×10 μm column; Mobile phase A: CO₂; Mobile phase B: (4:1, isopropyl alcohol: ACN)(0.1% NH₄OH); isocratic: 45% of Mobile Phase B; flow rate: 150 mL/min; column temperature: 40 °C; back pressure: 100 bar). The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic: 40% of Mobile Phase B; flow rate: 4.0 mL/min; backpressure: 1500 psi; column temperature: 35 °C to provide Example E268A: peak 1 ((SFC-MS) m/z (M+H)⁺=587.5 at retention time: 0.474 min, 100% ee) and Example E269A: peak 2 ((SFC-MS) m/z (M+H)⁺=587.5 at retention time: 1.916 min, 100% ee).

Example E280A and E281A: A mixture was separated into its component isomers using SFC purification (DAICEL CHIRALCEL OD, 250 mm×30 mm×10 μm column; Mobile phase A: CO₂; Mobile phase B: EtOH (0.1% NH₄OH); isocratic: 55% of Mobile Phase B; flow rate: 80 mL/min; column temperature: 40 °C; back pressure: 100 bar). The chiral purity was determined using SFC analytical method on Chiralcel OD-3 50 mm×4.6 mm×3 μm column; Mobile phase A: CO₂; Mobile phase B: EtOH (0.2% NH₃); isocratic: 40% of Mobile Phase B; flow rate: 4.0 mL/min; backpressure: 1500 psi; column temperature: 35 °C to provide Example E280A: peak 1 ((SFC-MS) m/z (M+H)+=574.5 at retention time: 1.470 min, 100% ee) and Example E281A: peak 2 ((SFC-MS) m/z (M+H)+=574.5 at retention time: 1.689 min, 98% ee).

Example E282A and E283A: A mixture was separated into its component isomers using SFC purification (DAICEL CHIRALPAK AD, 250 mm×30 mm×10 μm column; Mobile phase A: CO₂; Mobile phase B: EtOH (0.1% NH₄OH); isocratic: 50% of Mobile Phase B; flow rate: 120 mL/min; column temperature: 40 °C; back pressure: 100 bar). The chiral purity was determined using SFC analytical method on Chiralpak AD-3 50 mm×4.6 mm×3 μm column; Mobile phase A: CO₂; Mobile phase B: isopropyl alcohol (0.2% NH₃); isocratic: 40% of Mobile Phase B; flow rate: 3.5 mL/min; backpressure: 1500 psi; column temperature: 35 °C to provide Example E282A: peak 1 ((SFC-MS) m/z (M+H)⁺=586.6 at retention time: 1.035 min, 100% ee) and Example E283A: peak 2 ((SFC-MS) m/z (M+H)⁺=586.6 at retention time: 1.896 min, 99% ee).

Example E296A and E297A: A mixture was separated into its component isomers using SFC purification (DAICEL CHIRALCEL OD, 250 mm×30 mm×10 μm column; Mobile phase A: CO₂; Mobile phase B: EtOH (0.1% NH₄OH); isocratic: 55% of Mobile Phase B; flow rate: 80 mL/min; column temperature: 40 °C; back pressure: 100 bar). The chiral purity was determined using SFC analytical method on Chiralcel OD-3 50 mm×4.6 mm×3 μm column; Mobile phase A: CO₂; Mobile phase B: EtOH (0.2% NH₃); gradient: 5-40% of Mobile Phase B over 1.5 min then held at 40% of Mobile Phase B over 1.0 min then 5% of Mobile Phase B over 0.5 min; flow rate: 4.0 mL/min; backpressure: 1500 psi; column temperature: 35 °C to provide Example E296A: peak 1 ((SFC-MS) m/z (M+H)⁺=586.5 at retention time: 1.730 min, 100% ee) and Example E297A: peak 2 ((SFC-MS) m/z (M+H)⁺=586.5 at retention time: 2.068 min, 100% ee).

### Prophetic Examples

The compounds in Table 2 and Table 2A can be prepared using similar chemistry from Examples 1-758 that are commercially available or that can be synthesized by literature methods to form the resultant compounds of the disclosure. It is noted that where the stereochemistry of the compounds in table is not defined, the respective bonds can include S or R stereoisomers or can be a racemic mixture thereof.

TABLE 2

| Ex. | Structure | ChemDraw Name |
|---|---|---|
| V1 | 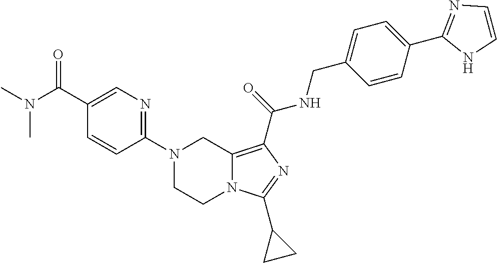 [SMILES: CN(C)C(=O)c1ccc(N2CCn3c(C4CC4)nc(C(=O)NCc4ccc(-c5ncc[nH]5)cc4)c3C2)nc1] | N-(4-(1H-imidazol-2-yl)benzyl)-3- cyclopropyl-7-(5- (dimethylcarbamoyl)pyridin-2-yl)- 5,6,7,8-tetrahydroimidazo[1,5- a]pyrazine-1-carboxamide |
| V2 | 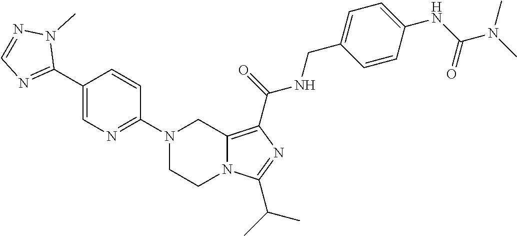 [SMILES: CC(C)c1nc(C(=O)NCc2ccc(NC(=O)N(C)C)cc2)c2n1CCN(c1ccc(-c3ncnn3C)cn1)C2] | N-(4-(3,3-dimethylureido)benzyl)-3- isopropyl-7-(5-(1-methyl-1H-1,2,4- triazol-5-yl)pyridin-2-yl)-5,6,7,8- tetrahydroimidazo[1,5-a]pyrazine-1- carboxamide |
| V3 | 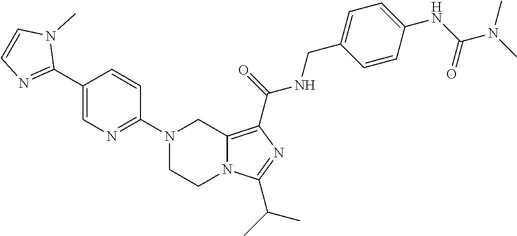 [SMILES: CC(C)c1nc(C(=O)NCc2ccc(NC(=O)N(C)C)cc2)c2n1CCN(c1ccc(-c3nccn3C)cn1)C2] | N-(4-(3,3-dimethylureido)benzyl)-3- isopropyl-7-(5-(1-methyl-1H- imidazol-2-yl)pyridin-2-yl)-5,6,7,8- tetrahydroimidazo[1,5-a]pyrazine-1- carboxamide |
| V4 | 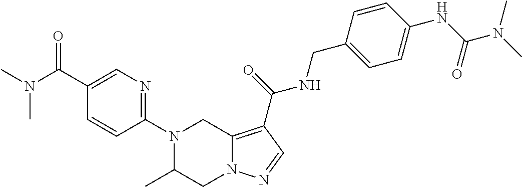 | 5-(5-(dimethylcarbamoyl)pyridin-2- yl)-N-(4-(3,3-dimethylureido)benzyl)- 6-methyl-4,5,6,7- tetrahydropyrazolo[1,5-a]pyrazine-3- carboxamide |
| V5 | 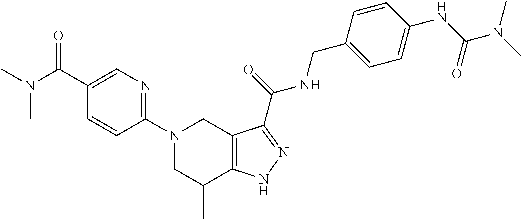 | 5-(5-(dimethylcarbamoyl)pyridin-2- yl)-N-(4-(3,3-dimethylureido)benzyl)- 7-methyl-4,5,6,7-tetrahydro-1H- pyrazolo[4,3-c]pyridine-3- carboxamide |
| V6 | 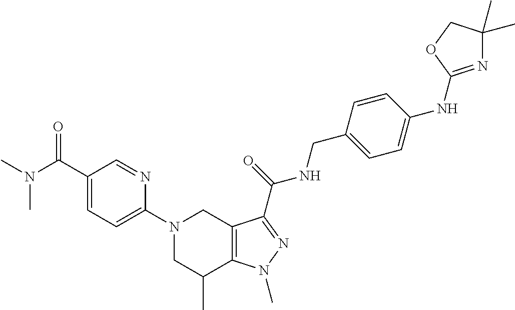 | N-(4-((4,4-dimethyl-4,5- dihydrooxazol-2-yl)amino)benzyl)-5- (5-(dimethylcarbamoyl)pyridin-2-yl)- 1-methyl-4,5,6,7-tetrahydro-1H- pyrazolo[4,3-c]pyridine-3- carboxamide |
| V7 | 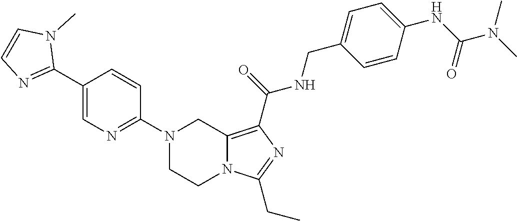 [SMILES: CCc1nc(C(=O)NCc2ccc(NC(=O)N(C)C)cc2)c2n1CCN(c1ccc(-c3nccn3C)cn1)C2] | N-(4-(3,3-dimethylureido)benzyl)-3- ethyl-7-(5-(1-methyl-1H-imidazol-2- yl)pyridin-2-yl)-5,6,7,8- tetrahydroimidazo[1,5-a]pyrazine-1- carboxamide |
| V8 | 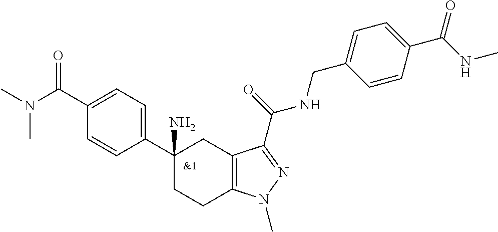 | rac-(R)-5-amino-5-(4- (dimethylcarbamoyl)phenyl)-1- methyl-N-(4- (methylcarbamoyl)benzyl)-4,5,6,7- tetrahydro-1H-indazole-3- carboxamide |
| V9 | 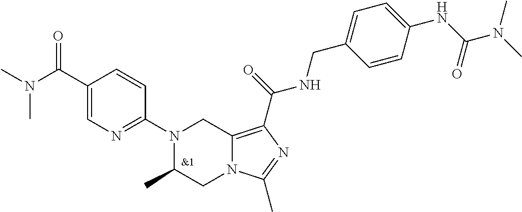 | rac-(R)-7-(5- (dimethylcarbamoyl)pyridin-2-yl)-N- (4-(3,3-dimethylureido)benzyl)-3,6- dimethyl-5,6,7,8- tetrahydroimidazo[1,5-a]pyrazine-1- carboxamide |
| V10 | 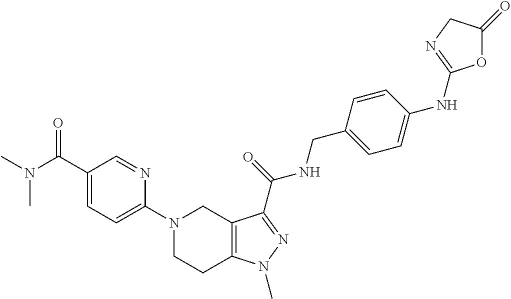 [SMILES: CN(C)C(=O)c1ccc(N2CCc3c(c(C(=O)NCc4ccc(NC5=NCC(=O)O5)cc4)nn3C)C2)nc1] | 5-(5-(dimethylcarbamoyl)pyridin-2- yl)-1-methyl-N-(4-((5-oxo-4,5- dihydrooxazol-2-yl)amino)benzyl)- 4,5,6,7-tetrahydro-1H-pyrazolo[4,3- c]pyridine-3-carboxamide |
| V11 | 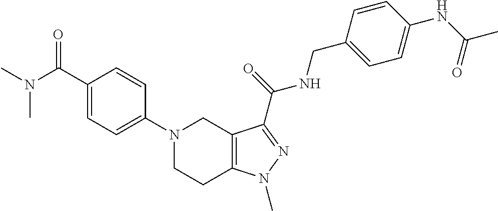 [SMILES: CC(=O)Nc1ccc(CNC(=O)c2nn(C)c3c2CN(c2ccc(C(=O)N(C)C)cc2)CC3)cc1] | N-(4-acetamidobenzyl)-5-(4- (dimethylcarbamoyl)phenyl)-1- methyl-4,5,6,7-tetrahydro-1H- pyrazolo[4,3-c]pyridine-3- carboxamide |
| V12 | 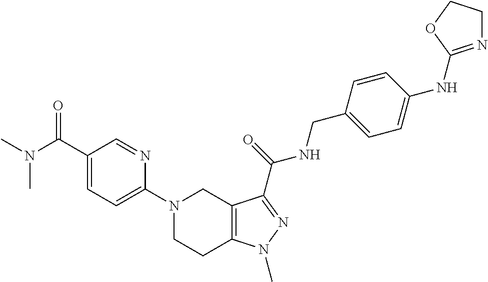 [SMILES: CN(C)C(=O)c1ccc(N2CCc3c(c(C(=O)NCc4ccc(NC5=NCCO5)cc4)nn3C)C2)nc1] | N-(4-((4,5-dihydrooxazol-2- yl)amino)benzyl)-5-(5- (dimethylcarbamoyl)pyridin-2-yl)-1- methyl-4,5,6,7-tetrahydro-1H- pyrazolo[4,3-c]pyridine-3- carboxamide |
| V13 | 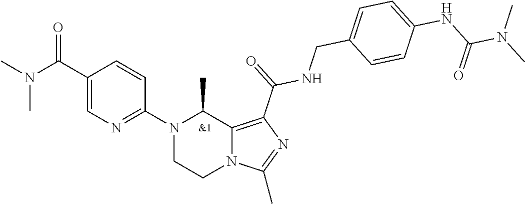 | rac-(R)-7-(5- (dimethylcarbamoyl)pyridin-2-yl)-N- (4-(3,3-dimethylureido)benzyl)-3,8- dimethyl-5,6,7,8- tetrahydroimidazo[1,5-a]pyrazine-1- carboxamide |
| V14 | 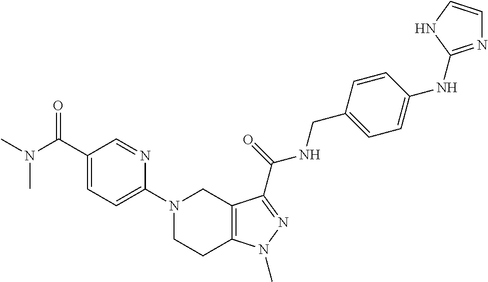 [SMILES: CN(C)C(=O)c1ccc(N2CCc3c(c(C(=O)NCc4ccc(Nc5ncc[nH]5)cc4)nn3C)C2)nc1] | N-(4-((1H-imidazol-2- yl)amino)benzyl)-5-(5- (dimethylcarbamoyl)pyridin-2-yl)-1- methyl-4,5,6,7-tetrahydro-1H- pyrazolo[4,3-c]pyridine-3- carboxamide |
| V15 | 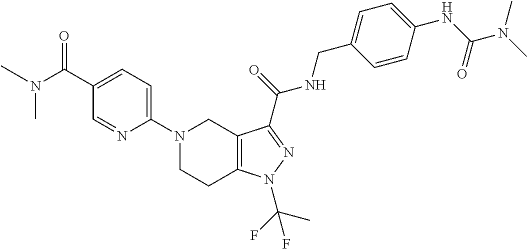 | 1-(1,1-difluoroethyl)-5-(5- (dimethylcarbamoyl)pyridin-2-yl)-N- (4-(3,3-dimethylureido)benzyl)- 4,5,6,7-tetrahydro-1H-pyrazolo[4,3- c]pyridine-3-carboxamide |
| V16 | 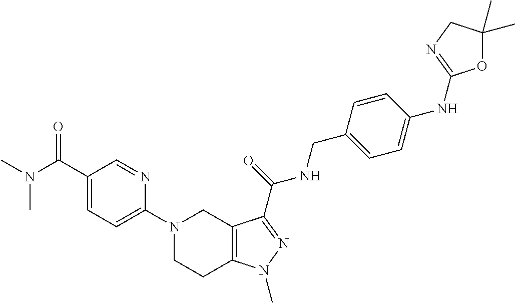 | N-(4-((5,5-dimethyl-4,5- dihydrooxazol-2-yl)amino)benzyl)-5- (5-(dimethylcarbamoyl)pyridin-2-yl)- 1-methyl-4,5,6,7-tetrahydro-1H- pyrazolo[4,3-c]pyridine-3- carboxamide |
| V17 | 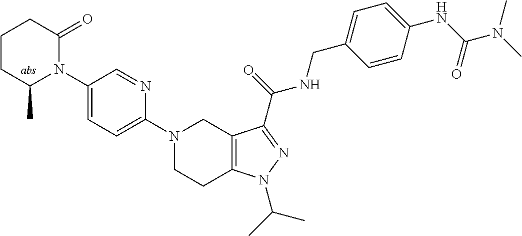 [SMILES: CC(C)n1nc(C(=O)NCc2ccc(NC(=O)N(C)C)cc2)c2c1CCN(c1ccc(N3C(=O)CCC[C@@H]3C)cn1)C2] | (S)-N-(4-(3,3-dimethylureido)benzyl)- 1-isopropyl-5-(5-(2-methyl-6- oxopiperidin-1-yl)pyridin-2-yl)- 4,5,6,7-tetrahydro-1H-pyrazolo[4,3- c]pyridine-3-carboxamide |
| V18 | 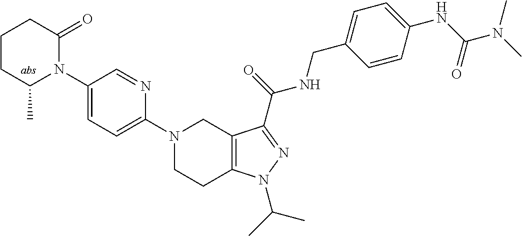 [SMILES: CC(C)n1nc(C(=O)NCc2ccc(NC(=O)N(C)C)cc2)c2c1CCN(c1ccc(N3C(=O)CCC[C@H]3C)cn1)C2] | (R)-N-(4-(3,3-dimethylureido)benzyl)- 1-isopropyl-5-(5-(2-methyl-6- oxopiperidin-1-yl)pyridin-2-yl)- 4,5,6,7-tetrahydro-1H-pyrazolo[4,3- c]pyridine-3-carboxamide |
| V19 | 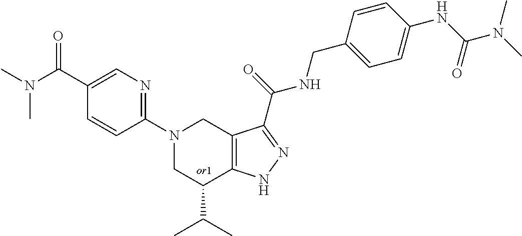 | rac-(R)-5-(5- (dimethylcarbamoyl)pyridin-2-yl)-N- (4-(3,3-dimethylureido)benzyl)-7- isopropyl-4,5,6,7-tetrahydro-1H- pyrazolo[4,3-c]pyridine-3- carboxamide |
| V20 | 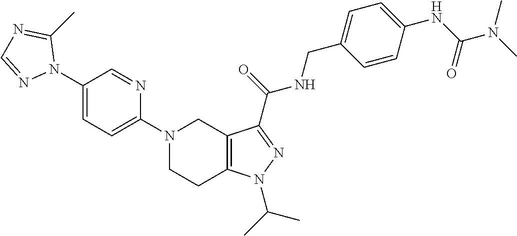 [SMILES: Cc1ncnn1-c1ccc(N2CCc3c(c(C(=O)NCc4ccc(NC(=O)N(C)C)cc4)nn3C(C)C)C2)nc1] | N-(4-(3,3-dimethylureido)benzyl)-1- isopropyl-5-(5-(5-methyl-1H-1,2,4- triazol-1-yl)pyridin-2-yl)-4,5,6,7- tetrahydro-1H-pyrazolo[4,3- c]pyridine-3-carboxamide |
| V21 | 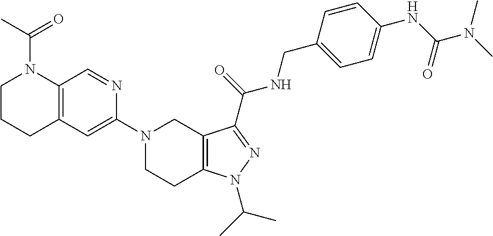 [SMILES: CC(=O)N1CCCc2cc(N3CCc4c(c(C(=O)NCc5ccc(NC(=O)N(C)C)cc5)nn4C(C)C)C3)ncc21] | 5-(1-acetyl-1,2,3,4-tetrahydro-1,7- naphthyridin-6-yl)-N-(4-(3,3- dimethylureido)benzyl)-1-isopropyl- 4,5,6,7-tetrahydro-1H-pyrazolo[4,3- c]pyridine-3-carboxamide |
| V22 | 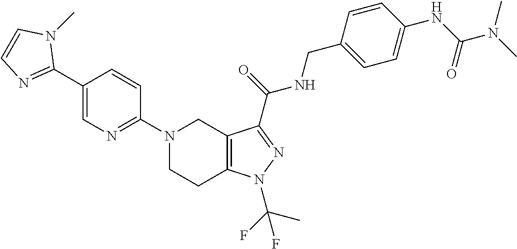 [SMILES: CN(C)C(=O)Nc1ccc(CNC(=O)c2nn(C(C)(F)F)c3c2CN(c2ccc(-c4nccn4C)cn2)CC3)cc1] | 3-(1,1-difluoroethyl)-N-(4-(3,3- dimethylureido)benzyl)-7-(5-(1- methyl-1H-imidazol-2-yl)pyridin-2-yl)- 5,6,7,8-tetrahydroimidazo[1,5- a]pyrazine-1-carboxamide |
| V23 | 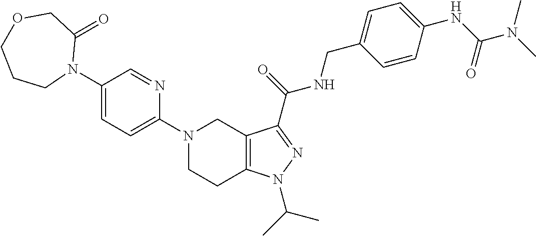 [SMILES: CC(C)n1nc(C(=O)NCc2ccc(NC(=O)N(C)C)cc2)c2c1CCN(c1ccc(N3CCCOCC3=O)cn1)C2] | N-(4-(3,3-dimethylureido)benzyl)-1- isopropyl-5-(5-(3-oxo-1,4-oxazepan- 4-yl)pyridin-2-yl)-4,5,6,7-tetrahydro- 1H-pyrazolo[4,3-c]pyridine-3- carboxamide |
| V24 | 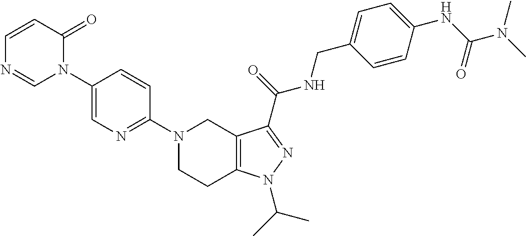 [SMILES: CC(C)n1nc(C(=O)NCc2ccc(NC(=O)N(C)C)cc2)c2c1CCN(c1ccc(-n3cnccc3=O)cn1)C2] | N-(4-(3,3-dimethylureido)benzyl)-1- isopropyl-5-(5-(6-oxopyrimidin-1(6H)- yl)pyridin-2-yl)-4,5,6,7-tetrahydro-1H- pyrazolo[4,3-c]pyridine-3- carboxamide |
| V25 | 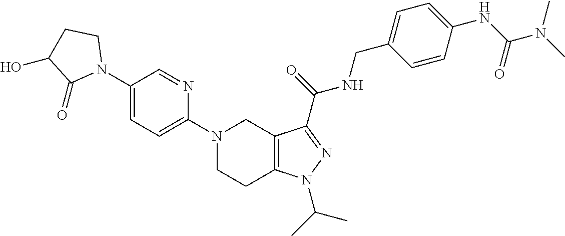 [SMILES: CC(C)n1nc(C(=O)NCc2ccc(NC(=O)N(C)C)cc2)c2c1CCN(c1ccc(N3CCC(O)C3=O)cn1)C2] | N-(4-(3,3-dimethylureido)benzyl)-5- (5-(3-hydroxy-2-oxopyrrolidin-1- yl)pyridin-2-yl)-1-isopropyl-4,5,6,7- tetrahydro-1H-pyrazolo[4,3- c]pyridine-3-carboxamide |
| V26 | 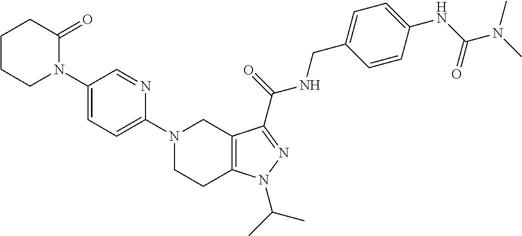 [SMILES: CC(C)n1nc(C(=O)NCc2ccc(NC(=O)N(C)C)cc2)c2c1CCN(c1ccc(N3CCCCC3=O)cn1)C2] | N-(4-(3,3-dimethylureido)benzyl)-1- isopropyl-5-(5-(2-oxopiperidin-1- yl)pyridin-2-yl)-4,5,6,7-tetrahydro-1H- pyrazolo[4,3-c]pyridine-3- carboxamide |
| V27 | 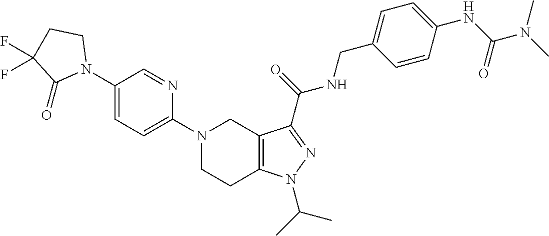 [SMILES: CC(C)n1nc(C(=O)NCc2ccc(NC(=O)N(C)C)cc2)c2c1CCN(c1ccc(N3CCC(F)(F)C3=O)cn1)C2] | 5-(5-(3,3-difluoro-2-oxopyrrolidin-1- yl)pyridin-2-yl)-N-(4-(3,3- dimethylureido)benzyl)-1-isopropyl- 4,5,6,7-tetrahydro-1H-pyrazolo[4,3- c]pyridine-3-carboxamide |
| V28 | 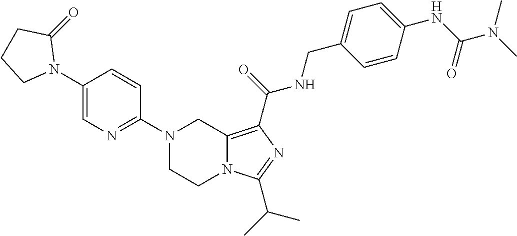 [SMILES: CC(C)c1nc(C(=O)NCc2ccc(NC(=O)N(C)C)cc2)c2n1CCN(c1ccc(N3CCCC3=O)cn1)C2] | N-(4-(3,3-dimethylureido)benzyl)-3- isopropyl-7-(5-(2-oxopyrrolidin-1- yl)pyridin-2-yl)-5,6,7,8- tetrahydroimidazo[1,5-a]pyrazine-1- carboxamide |
| V29 | 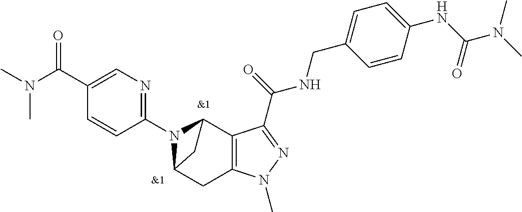 | rac-(4R,6S)-8-(5- (dimethylcarbamoyl)pyridin-2-yl)-N- (4-(3,3-dimethylureido)benzyl)-1- methyl-4,5,6,7-tetrahydro-1H-4,6- epiminoindazole-3-carboxamide |
| V30 | 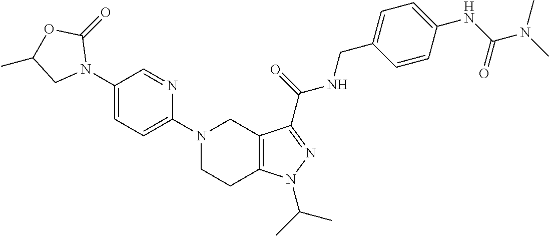 | N-(4-(3,3-dimethylureido)benzyl)-1- isopropyl-5-(5-(5-methyl-2- oxooxazolidin-3-yl)pyridin-2-yl)- 4,5,6,7-tetrahydro-1H-pyrazolo[4,3- c]pyridine-3-carboxamide |
| V31 | 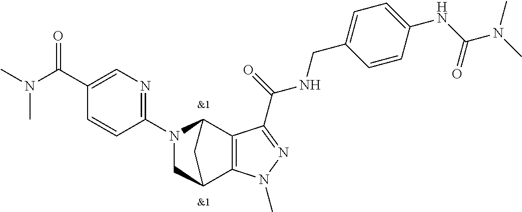 | rac-(4R,7R)-5-(5- (dimethylcarbamoyl)pyridin-2-yl)-N- (4-(3,3-dimethylureido)benzyl)-1- methyl-4,5,6,7-tetrahydro-1H-4,7- methanopyrazolo[4,3-c]pyridine-3- carboxamide |
| V32 | 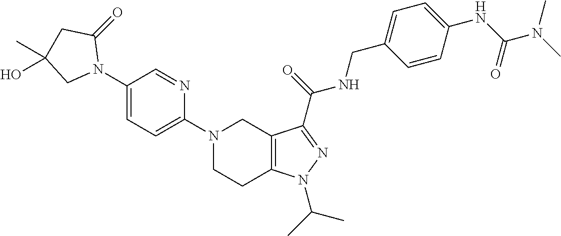 | N-(4-(3,3-dimethylureido)benzyl)-5- (5-(4-hydroxy-4-methyl-2- oxopyrrolidin-1-yl)pyridin-2-yl)-1- isopropyl-4,5,6,7-tetrahydro-1H- pyrazolo[4,3-c]pyridine-3- carboxamide |
| V33 | 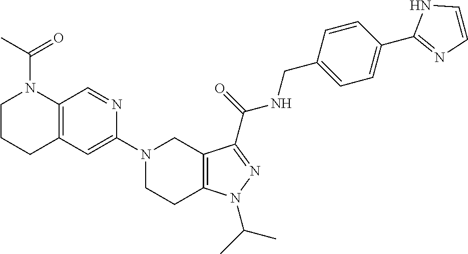 [SMILES: CC(=O)N1CCCc2cc(N3CCc4c(c(C(=O)NCc5ccc(-c6ncc[nH]6)cc5)nn4C(C)C)C3)ncc21] | N-(4-(1H-imidazol-2-yl)benzyl)-5-(1- acetyl-1,2,3,4-tetrahydro-1,7- naphthyridin-6-yl)-1-isopropyl- 4,5,6,7-tetrahydro-1H-pyrazolo[4,3- c]pyridine-3-carboxamide |
| V34 | 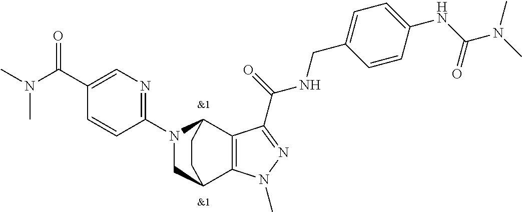 | rac-(4R,7R)-9-(5- (dimethylcarbamoyl)pyridin-2-yl)-N- (4-(3,3-dimethylureido)benzyl)-1- methyl-4,5,6,7-tetrahydro-1H-4,7- (epiminomethano)indazole-3- carboxamide |
| V35 | 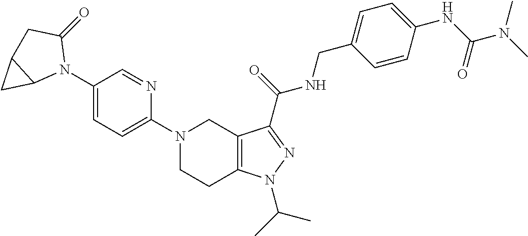 [SMILES: CC(C)n1nc(C(=O)NCc2ccc(NC(=O)N(C)C)cc2)c2c1CCN(c1ccc(N3C(=O)CC4CC43)cn1)C2] | N-(4-(3,3-dimethylureido)benzyl)-1- isopropyl-5-(5-(3-oxo-2- azabicyclo[3.1.0]hexan-2-yl)pyridin- 2-yl)-4,5,6,7-tetrahydro-1H- pyrazolo[4,3-c]pyridine-3- carboxamide |
| V36 | 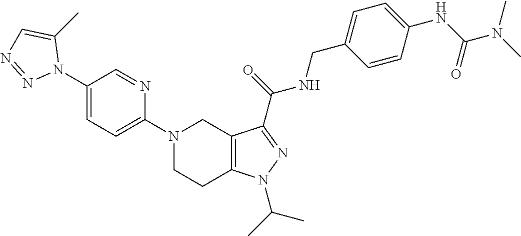 [SMILES: Cc1cnnn1-c1ccc(N2CCc3c(c(C(=O)NCc4ccc(NC(=O)N(C)C)cc4)nn3C(C)C)C2)nc1] | N-(4-(3,3-dimethylureido)benzyl)-1- isopropyl-5-(5-(5-methyl-1H-1,2,3- triazol-1-yl)pyridin-2-yl)-4,5,6,7- tetrahydro-1H-pyrazolo[4,3- c]pyridine-3-carboxamide |
| V37 | 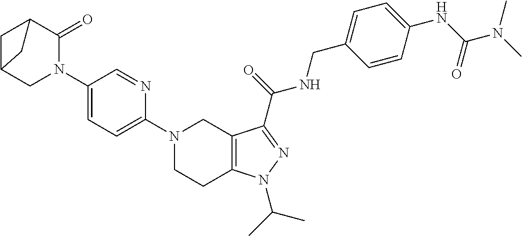 [SMILES: CC(C)n1nc(C(=O)NCc2ccc(NC(=O)N(C)C)cc2)c2c1CCN(c1ccc(N3CC4CC(C4)C3=O)cn1)C2] | N-(4-(3,3-dimethylureido)benzyl)-1- isopropyl-5-(5-(2-oxo-3- azabicyclo[3.1.1]heptan-3-yl)pyridin- 2-yl)-4,5,6,7-tetrahydro-1H- pyrazolo[4,3-c]pyridine-3- carboxamide |
| V38 | 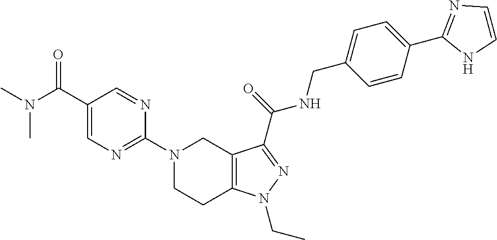 [SMILES: CCn1nc(C(=O)NCc2ccc(-c3ncc[nH]3)cc2)c2c1CCN(c1ncc(C(=O)N(C)C)cn1)C2] | N-(4-(1H-imidazol-2-yl)benzyl)-5-(5- (dimethylcarbamoyl)pyrimidin-2-yl)-1- ethyl-4,5,6,7-tetrahydro-1H- pyrazolo[4,3-c]pyridine-3- carboxamide |
| V39 | 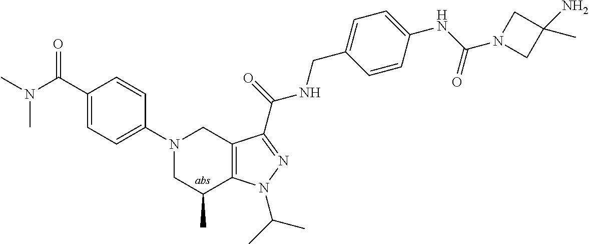 | (S)-N-(4-(3-amino-3-methylazetidine- 1-carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V40 | 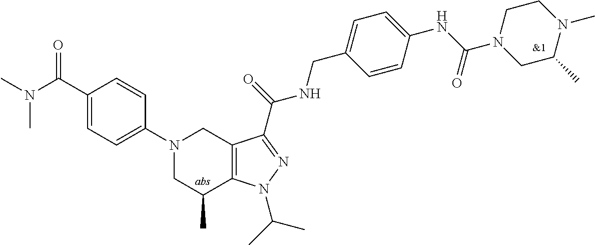 | (S)-5-(4-(dimethylcarbamoyl)phenyl)- N-(4-((RS)-3,4-dimethylpiperazine-1- carboxamido)benzyl)-1-isopropyl-7- methyl-4,5,6,7-tetrahydro-1H- pyrazolo[4,3-c]pyridine-3- carboxamide |
| V41 | 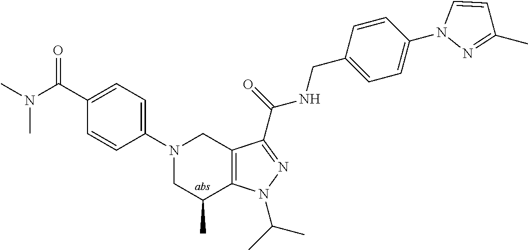 | (S)-5-(4-(dimethylcarbamoyl)phenyl)- 1-isopropyl-7-methyl-N-(4-(3-methyl- 1H-pyrazol-1-yl)benzyl)-4,5,6,7- tetrahydro-1H-pyrazolo[4,3- c]pyridine-3-carboxamide |
| V42 | 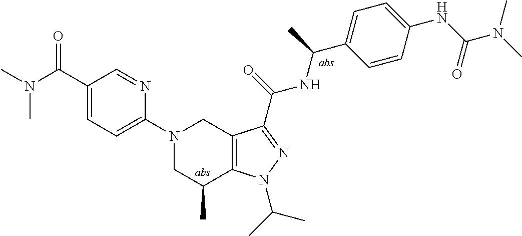 | (S)-5-(5-(dimethylcarbamoyl)pyridin- 2-yl)-N-((S)-1-(4-(3,3- dimethylureido)phenyl)ethyl)-1- isopropyl-7-methyl-4,5,6,7- tetrahydro-1H-pyrazolo[4,3- c]pyridine-3-carboxamide |
| V43 | 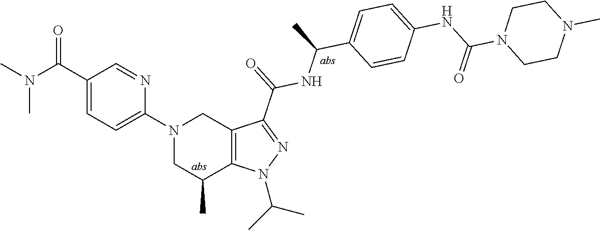 | (S)-5-(5-(dimethylcarbamoyl)pyridin- 2-yl)-1-isopropyl-7-methyl-N-((S)-1- (4-(4-methylpiperazine-1- carboxamido)phenyl)ethyl)-4,5,6,7- tetrahydro-1H-pyrazolo[4,3- c]pyridine-3-carboxamide |
| V44 | 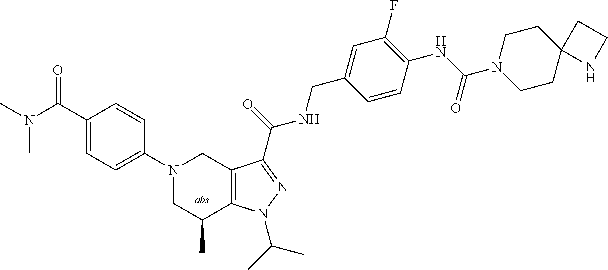 [SMILES: CC(C)n1nc(C(=O)NCc2ccc(NC(=O)N3CCC4(CCN4)CC3)c(F)c2)c2c1[C@@H](C)CN(c1ccc(C(=O)N(C)C)cc1)C2] | (S)-5-(4-(dimethylcarbamoyl)phenyl)- N-(3-fluoro-4-(1,7- diazaspiro[3.5]nonane-7- carboxamido)benzyl)-1-isopropyl-7- methyl-4,5,6,7-tetrahydro-1H- pyrazolo[4,3-c]pyridine-3- carboxamide |
| V45 | 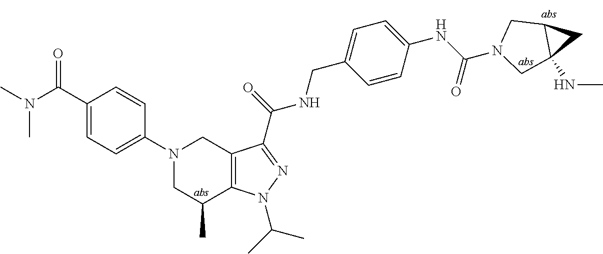 | (S)-5-(4-(dimethylcarbamoyl)phenyl)- 1-isopropyl-7-methyl-N-(4-((1S,5R)- 1-(methylamino)-3- azabicyclo[3.1.0]hexane-3- carboxamido)benzyl)-4,5,6,7- tetrahydro-1H-pyrazolo[4,3- c]pyridine-3-carboxamide |
| V46 | 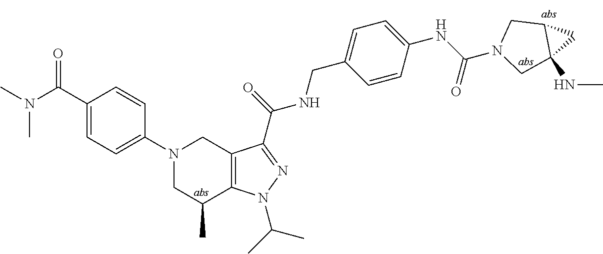 | (S)-5-(4-(dimethylcarbamoyl)phenyl)- 1-isopropyl-7-methyl-N-(4-((1R,5S)- 1-(methylamino)-3- azabicyclo[3.1.0]hexane-3- carboxamido)benzyl)-4,5,6,7- tetrahydro-1H-pyrazolo[4,3- c]pyridine-3-carboxamide |
| V47 | 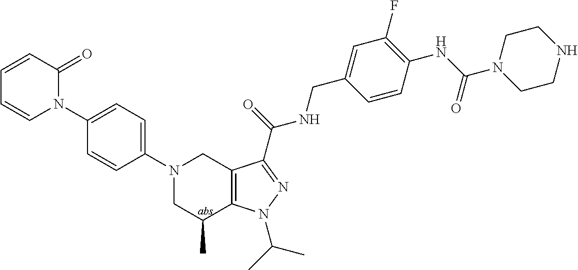 [SMILES: CC(C)n1nc(C(=O)NCc2ccc(NC(=O)N3CCNCC3)c(F)c2)c2c1[C@@H](C)CN(c1ccc(-n3ccccc3=O)cc1)C2] | (S)-N-(3-fluoro-4-(piperazine-1- carboxamido)benzyl)-1-isopropyl-7- methyl-5-(4-(2-oxopyridin-1(2H)- yl)phenyl)-4,5,6,7-tetrahydro-1H- pyrazolo[4,3-c]pyridine-3- carboxamide |
| V48 | 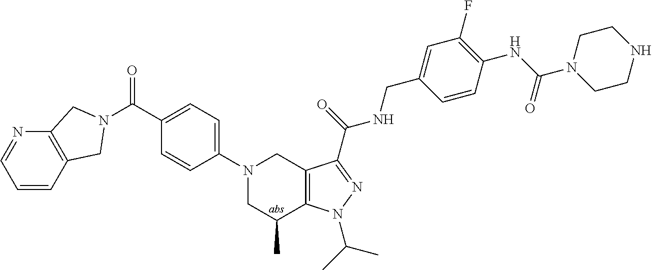 [SMILES: CC(C)n1nc(C(=O)NCc2ccc(NC(=O)N3CCNCC3)c(F)c2)c2c1[C@@H](C)CN(c1ccc(C(=O)N3Cc4cccnc4C3)cc1)C2] | (S)-5-(4-(6,7-dihydro-5H-pyrrolo[3,4- b]pyridine-6-carbonyl)phenyl)-N-(3- fluoro-4-(piperazine-1- carboxamido)benzyl)-1-isopropyl-7- methyl-4,5,6,7-tetrahydro-1H- pyrazolo[4,3-c]pyridine-3- carboxamide |
| V49 | 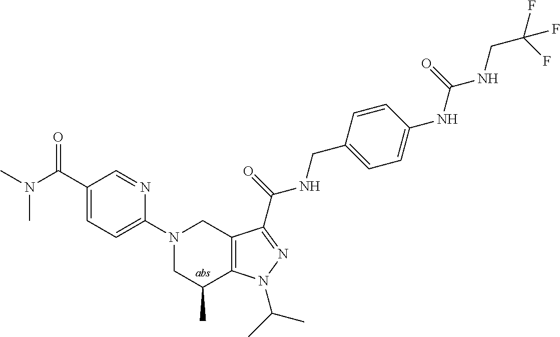 [SMILES: CC(C)n1nc(C(=O)NCc2ccc(NC(=O)NCC(F)(F)F)cc2)c2c1[C@@H](C)CN(c1ccc(C(=O)N(C)C)cn1)C2] | (S)-5-(5-(dimethylcarbamoyl)pyridin- 2-yl)-1-isopropyl-7-methyl-N-(4-(3- (2,2,2-trifluoroethyl)ureido)benzyl)- 4,5,6,7-tetrahydro-1H-pyrazolo[4,3- c]pyridine-3-carboxamide |
| V50 | 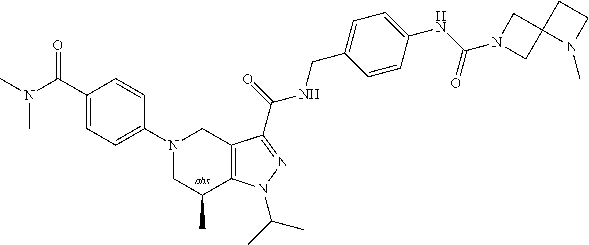 [SMILES: CC(C)n1nc(C(=O)NCc2ccc(NC(=O)N3CC4(CCN4C)C3)cc2)c2c1[C@@H](C)CN(c1ccc(C(=O)N(C)C)cc1)C2] | (S)-5-(4-(dimethylcarbamoyl)phenyl)- 1-isopropyl-7-methyl-N-(4-(1-methyl- 1,6-diazaspiro[3.3]heptane-6- carboxamido)benzyl)-4,5,6,7- tetrahydro-1H-pyrazolo[4,3- c]pyridine-3-carboxamide |
| V51 | 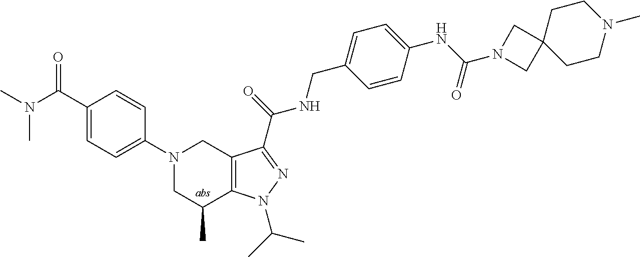 | (S)-5-(4-(dimethylcarbamoyl)phenyl)- 1-isopropyl-7-methyl-N-(4-(7-methyl- 2,7-diazaspiro[3.5]nonane-2- carboxamido)benzyl)-4,5,6,7- tetrahydro-1H-pyrazolo[4,3- c]pyridine-3-carboxamide |
| V52 | 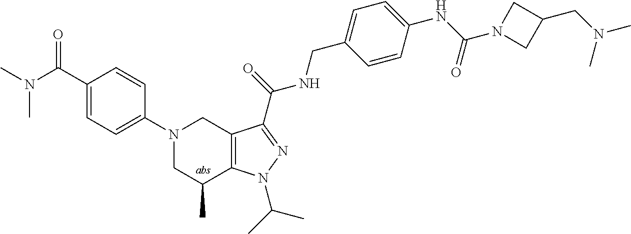 | (S)-N-(4-(3- ((dimethylamino)methyl)azetidine-1- carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V53 | 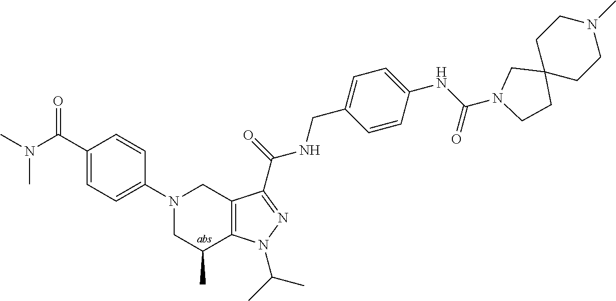 | (S)-N-(4-((5-(4- (dimethylcarbamoyl)phenyl)-1- isopropyl-7-methyl-4,5,6,7- tetrahydro-1H-pyrazolo[4,3- c]pyridine-3- carboxamido)methyl)phenyl)-8- methyl-2,8-diazaspiro[4.5]decane-2- carboxamide |
| V54 | 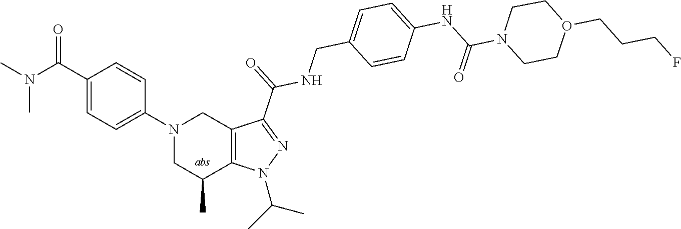 | (S)-5-(4-(dimethylcarbamoyl)phenyl)- N-(4-(4-(3-fluoropropoxy)piperidine- 1-carboxamido)benzyl)-1-isopropyl- 7-methyl-4,5,6,7-tetrahydro-1H- pyrazolo[4,3-c]pyridine-3- carboxamide |
| V55 | 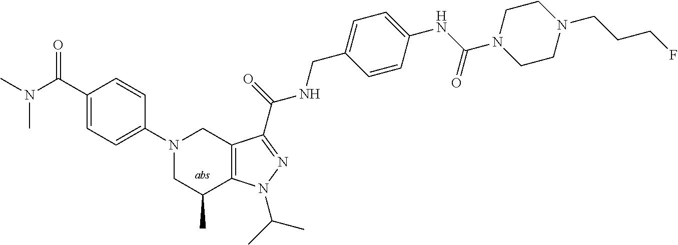 [SMILES: CC(C)n1nc(C(=O)NCc2ccc(NC(=O)N3CCN(CCCF)CC3)cc2)c2c1[C@@H](C)CN(c1ccc(C(=O)N(C)C)cc1)C2] | (S)-5-(4-(dimethylcarbamoyl)phenyl)- N-(4-(4-(3-fluoropropyl)piperazine-1- carboxamido)benzyl)-1-isopropyl-7- methyl-4,5,6,7-tetrahydro-1H- pyrazolo[4,3-c]pyridine-3- carboxamide |
| V56 | 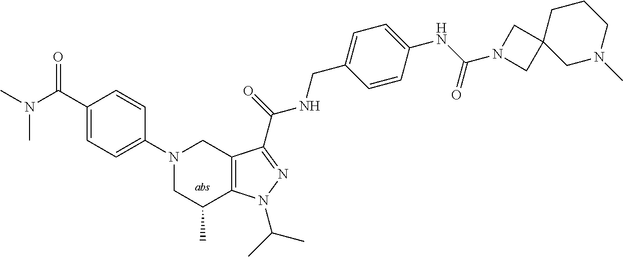 | (S)-N-(4-((5-(4- (dimethylcarbamoyl)phenyl)-1- isopropyl-7-methyl-4,5,6,7- tetrahydro-1H-pyrazolo[4,3- c]pyridine-3- carboxamido)methyl)phenyl)-8- methyl-5-oxa-2,8- diazaspiro[3.5]nonane-2- carboxamide |
| V57 | 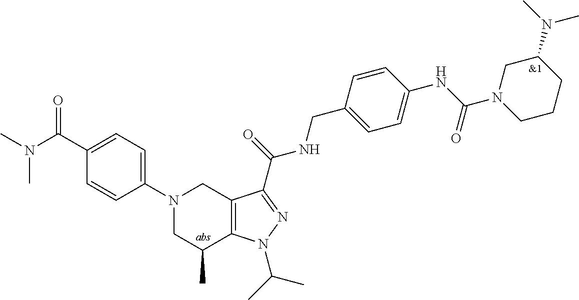 | (S)-N-(4-((RS)-3- (dimethylamino)piperidine-1- carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V58 | 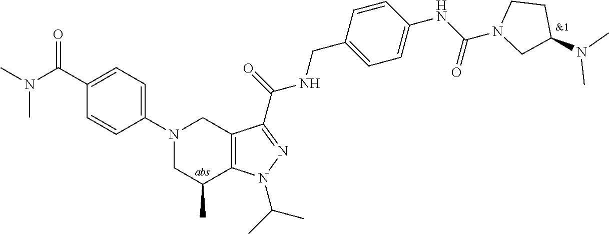 | (S)-N-(4-((RS)-3- (dimethylamino)pyrrolidine-1- carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V59 | 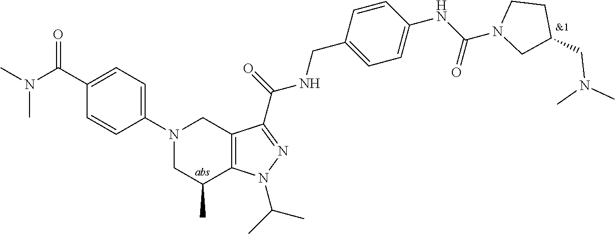 | (S)-N-(4-((RS)-3- ((dimethylamino)methyl)pyrrolidine- 1-carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V60 | 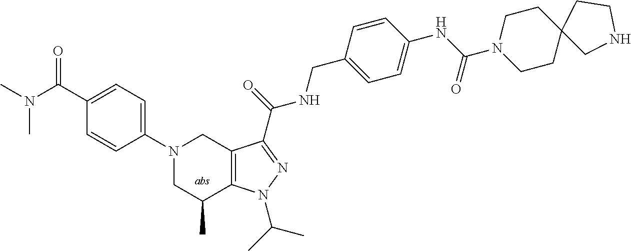 [SMILES: CC(C)n1nc(C(=O)NCc2ccc(NC(=O)N3CCC4(CCNC4)CC3)cc2)c2c1[C@@H](C)CN(c1ccc(C(=O)N(C)C)cc1)C2] | (S)-N-(4-((5-(4- (dimethylcarbamoyl)phenyl)-1- isopropyl-7-methyl-4,5,6,7- tetrahydro-1H-pyrazolo[4,3- c]pyridine-3- carboxamido)methyl)phenyl)-2,8- diazaspiro[4.5]decane-8- carboxamide |
| V61 | 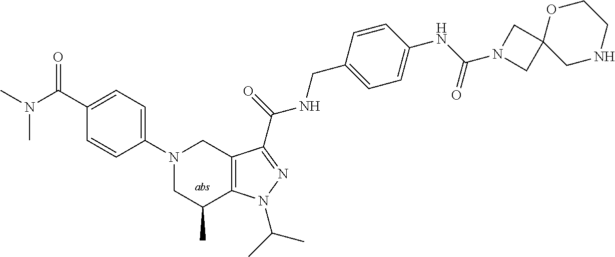 [SMILES: CC(C)n1nc(C(=O)NCc2ccc(NC(=O)N3CC4(CNCCO4)C3)cc2)c2c1[C@@H](C)CN(c1ccc(C(=O)N(C)C)cc1)C2] | (S)-N-(4-((5-(4- (dimethylcarbamoyl)phenyl)-1- isopropyl-7-methyl-4,5,6,7- tetrahydro-1H-pyrazolo[4,3- c]pyridine-3- carboxamido)methyl)phenyl)-5-oxa- 2,8-diazaspiro[3.5]nonane-2- carboxamide |
| V62 | 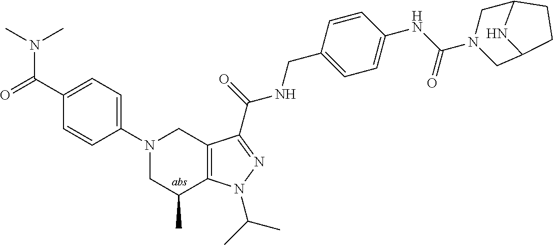 [SMILES: CC(C)n1nc(C(=O)NCc2ccc(NC(=O)N3CC4CCC(C3)N4)cc2)c2c1[C@@H](C)CN(c1ccc(C(=O)N(C)C)cc1)C2] | (7S)-N-(4-(3,8- diazabicyclo[3.2.1]octane-3- carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V63 | 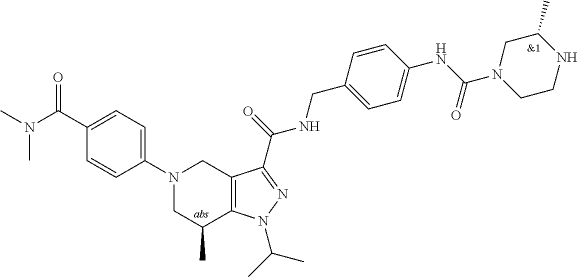 [SMILES: CC(C)n1nc(C(=O)NCc2ccc(NC(=O)N3CCN[C@@H](C)C3)cc2)c2c1[C@@H](C)CN(c1ccc(C(=O)N(C)C)cc1)C2] | (S)-5-(4-(dimethylcarbamoyl)phenyl)- 1-isopropyl-7-methyl-N-(4-((RS)-3- methylpiperazine-1- carboxamido)benzyl)-4,5,6,7- tetrahydro-1H-pyrazolo[4,3- c]pyridine-3-carboxamide |
| V64 | 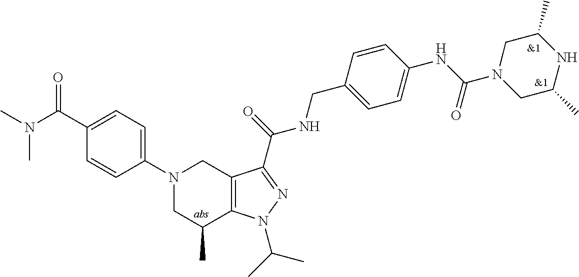 | (S)-5-(4-(dimethylcarbamoyl)phenyl)- N-(4-((3RS,5SR)-3,5- dimethylpiperazine-1- carboxamido)benzyl)-1-isopropyl-7- methyl-4,5,6,7-tetrahydro-1H- pyrazolo[4,3-c]pyridine-3- carboxamide |
| V65 | 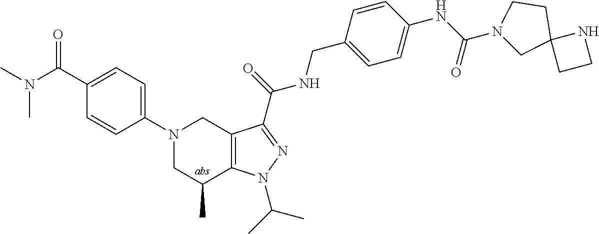 [SMILES: CC(C)n1nc(C(=O)NCc2ccc(NC(=O)N3CCC4(CCN4)C3)cc2)c2c1[C@@H](C)CN(c1ccc(C(=O)N(C)C)cc1)C2] | (7S)-N-(4-(1,6-diazaspiro[3.4]octane- 6-carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V66 | 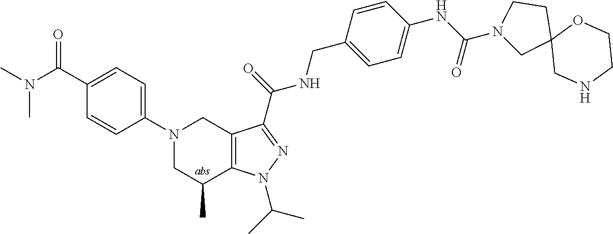 [SMILES: CC(C)n1nc(C(=O)NCc2ccc(NC(=O)N3CCC4(CNCCO4)C3)cc2)c2c1[C@@H](C)CN(c1ccc(C(=O)N(C)C)cc1)C2] | N-(4-(((S)-5-(4- (dimethylcarbamoyl)phenyl)-1- isopropyl-7-methyl-4,5,6,7- tetrahydro-1H-pyrazolo[4,3- c]pyridine-3- carboxamido)methyl)phenyl)-6-oxa- 2,9-diazaspiro[4.5]decane-2- carboxamide |
| V67 | 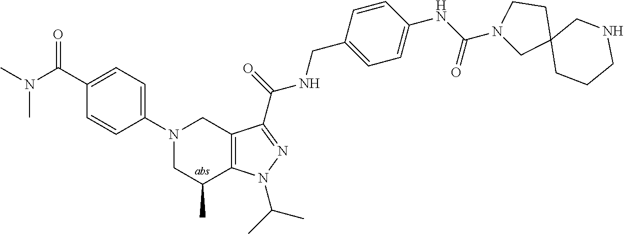 [SMILES: CC(C)n1nc(C(=O)NCc2ccc(NC(=O)N3CCC4(CCCNC4)C3)cc2)c2c1[C@@H](C)CN(c1ccc(C(=O)N(C)C)cc1)C2] | N-(4-(((S)-5-(4- (dimethylcarbamoyl)phenyl)-1- isopropyl-7-methyl-4,5,6,7- tetrahydro-1H-pyrazolo[4,3- c]pyridine-3- carboxamido)methyl)phenyl)-2,7- diazaspiro[4.5]decane-2- carboxamide |
| V68 | 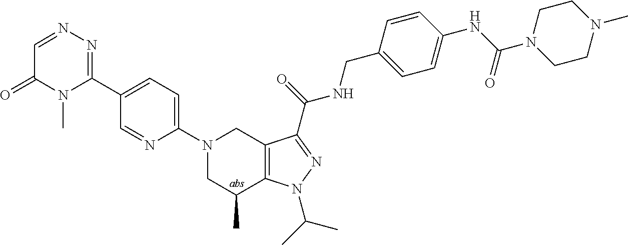 [SMILES: CC(C)n1nc(C(=O)NCc2ccc(NC(=O)N3CCN(C)CC3)cc2)c2c1[C@@H](C)CN(c1ccc(-c3nncc(=O)n3C)cn1)C2] | (S)-1-isopropyl-7-methyl-5-(5-(4- methyl5-oxo-4,5-dihydro-1,2,4- triazin-3-yl)pyridin-2-yl)-N-(4-(4- methylpiperazine-1- carboxamido)benzyl)-4,5,6,7- tetrahydro-1H-pyrazolo[4,3- c]pyridine-3-carboxamide |
| V69 | 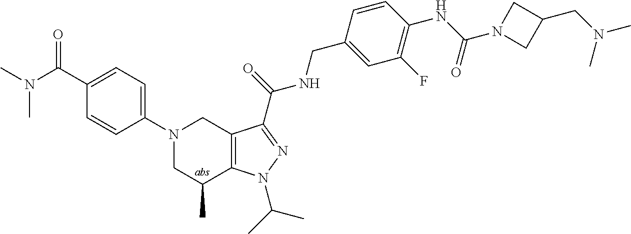 | (S)-N-(4-(3- ((dimethylamino)methyl)azetidine-1- carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V70 | 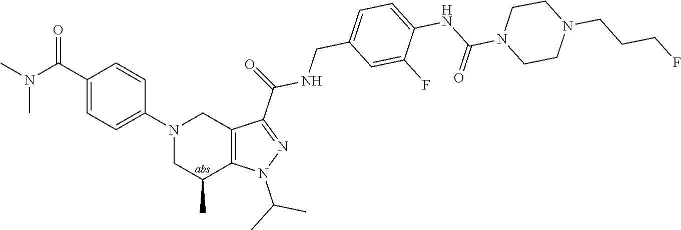 [SMILES: CC(C)n1nc(C(=O)NCc2ccc(NC(=O)N3CCN(CCCF)CC3)c(F)c2)c2c1[C@@H](C)CN(c1ccc(C(=O)N(C)C)cc1)C2] | (S)-5-(4-(dimethylcarbamoyl)phenyl)- N-(3-fluoro-4-(4-(3- fluoropropyl)piperazine-1- carboxamido)benzyl)-1-isopropyl-7- methyl-4,5,6,7-tetrahydro-1H- pyrazolo[4,3-c]pyridine-3- carboxamide |
| V71 | 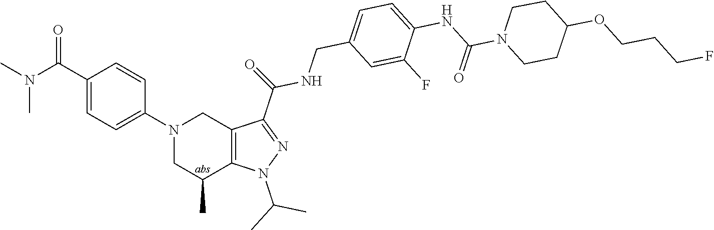 [SMILES: CC(C)n1nc(C(=O)NCc2ccc(NC(=O)N3CCC(OCCCF)CC3)c(F)c2)c2c1[C@@H](C)CN(c1ccc(C(=O)N(C)C)cc1)C2] | (S)-5-(4-(dimethylcarbamoyl)phenyl)- N-(3-fluoro-4-(4-(3- fluoropropoxy)piperidine-1- carboxamido)benzyl)-1-isopropyl-7- methyl-4,5,6,7-tetrahydro-1H- pyrazolo[4,3-c]pyridine-3- carboxamide |
| V72 | 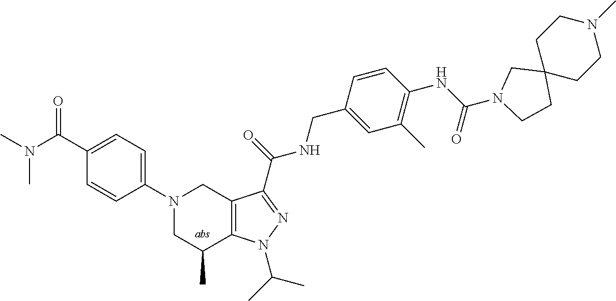 | (S)-N-(4-((5-(4- (dimethylcarbamoyl)phenyl)-1- isopropyl-7-methyl-4,5,6,7- tetrahydro-1H-pyrazolo[4,3- c]pyridine-3-carboxamido)methyl)-2- fluorophenyl)-8-methyl-2,8- diazaspiro[4.5]decane-2- carboxamide |
| V73 | 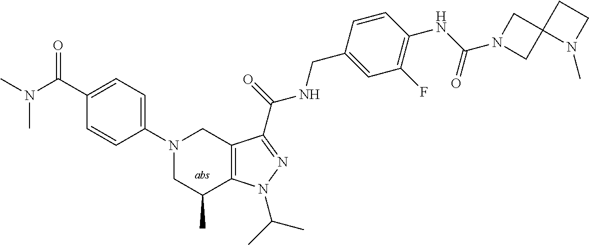 [SMILES: CC(C)n1nc(C(=O)NCc2ccc(NC(=O)N3CC4(CCN4C)C3)c(F)c2)c2c1[C@@H](C)CN(c1ccc(C(=O)N(C)C)cc1)C2] | (S)-5-(4-(dimethylcarbamoyl)phenyl)- N-(3-fluoro-4-(1-methyl-1,6- diazaspiro[3.3]heptane-6- carboxamido)benzyl)-1-isopropyl-7- methyl-4,5,6,7-tetrahydro-1H- pyrazolo[4,3-c]pyridine-3- carboxamide |
| V74 | 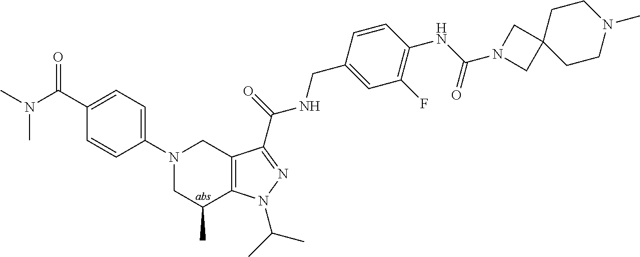 | (S)-5-(4-(dimethylcarbamoyl)phenyl)- N-(3-fluoro-4-(7-methyl-2,7- diazaspiro[3.5]nonane-2- carboxamido)benzyl)-1-isopropyl-7- methyl-4,5,6,7-tetrahydro-1H- pyrazolo[4,3-c]pyridine-3- carboxamide |
| V75 | 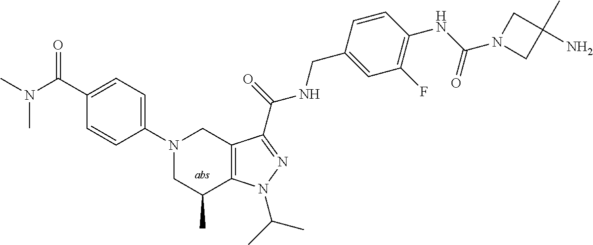 [SMILES: CC(C)n1nc(C(=O)NCc2ccc(NC(=O)N3CC(C)(N)C3)c(F)c2)c2c1[C@@H](C)CN(c1ccc(C(=O)N(C)C)cc1)C2] | (S)-N-(4-(3-amino-3-methylazetidine- 1-carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V76 | 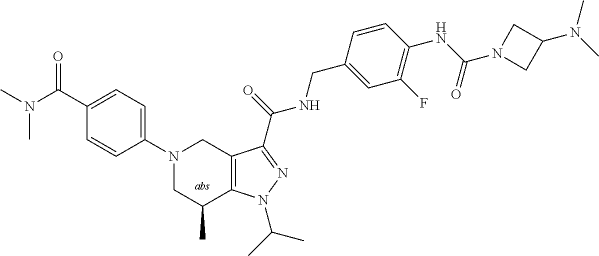 | (S)-N-(4-(3- (dimethylamino)azetidine-1- carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V77 | 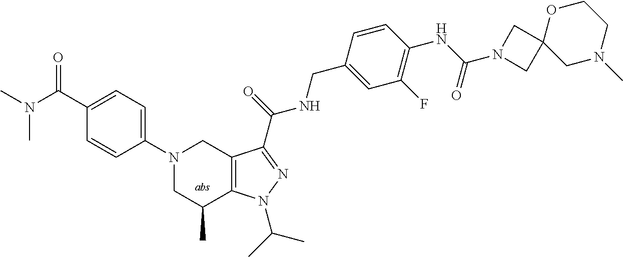 | (S)-N-(4-((5-(4- (dimethylcarbamoyl)phenyl)-1- isopropyl-7-methyl-4,5,6,7- tetrahydro-1H-pyrazolo[4,3- c]pyridine-3-carboxamido)methyl)-2- fluorophenyl)-8-methyl-5-oxa-2,8- diazaspiro[3.5]nonane-2- carboxamide |
| V78 | 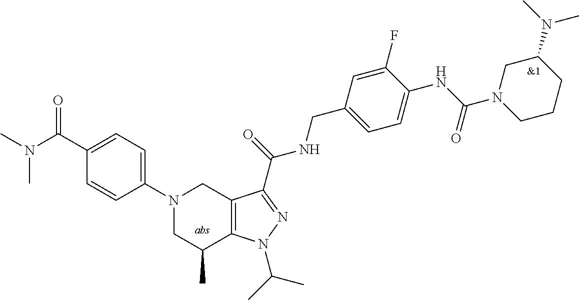 | (S)-N-(4-((RS)-3- (dimethylamino)piperidine-1- carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V79 | 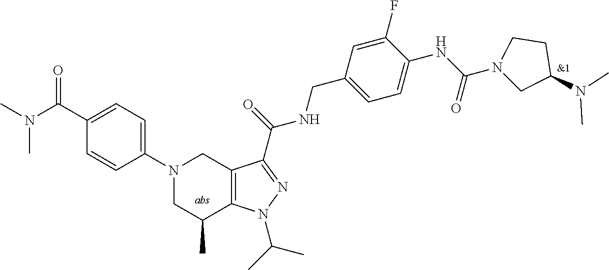 | (S)-N-(4-((RS)-3- (dimethylamino)pyrrolidine-1- carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V80 | 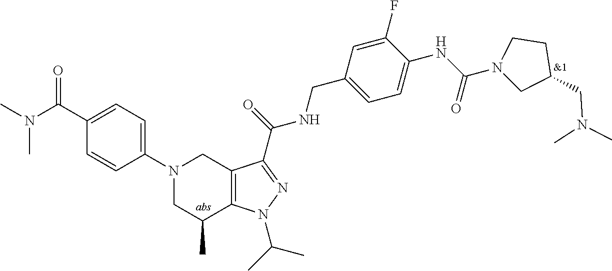 | (S)-N-(4-((RS)-3- ((dimethylamino)methyl)pyrrolidine- 1-carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V81 | 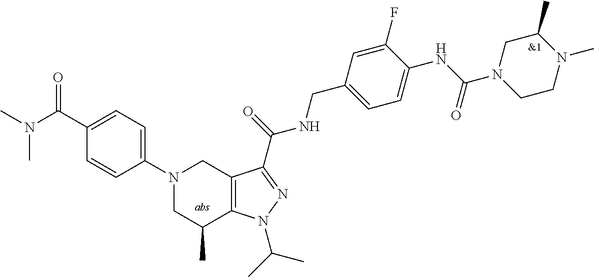 | (S)-5-(4-(dimethylcarbamoyl)phenyl)- N-(4-((RS)-3,4-dimethylpiperazine-1- carboxamido)-3-fluorobenzyl)-1- isopropyl-7-methyl-4,5,6,7- tetrahydro-1H-pyrazolo[4,3- c]pyridine-3-carboxamide |
| V82 | 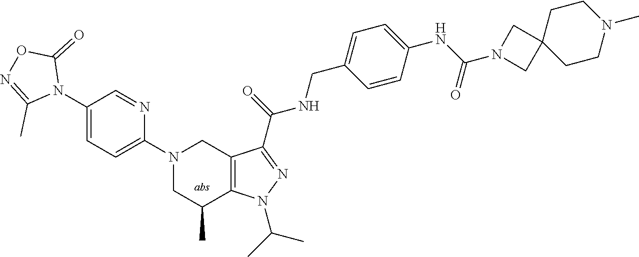 [SMILES: Cc1noc(=O)n1-c1ccc(N2Cc3c(C(=O)NCc4ccc(NC(=O)N5CC6(CCN(C)CC6)C5)cc4)nn(C(C)C)c3[C@@H](C)C2)nc1] | (S)-1-isopropyl-7-methyl-N-(4-(7- methyl-2,7-diazaspiro[3.5]nonane-2- carboxamido)benzyl)-5-(5-(3-methyl- 5-oxo-1,2,4-oxadiazol-4(5H)- yl)pyridin-2-yl)-4,5,6,7-tetrahydro-1H- pyrazolo[4,3-c]pyridine-3- carboxamide |
| V83 | 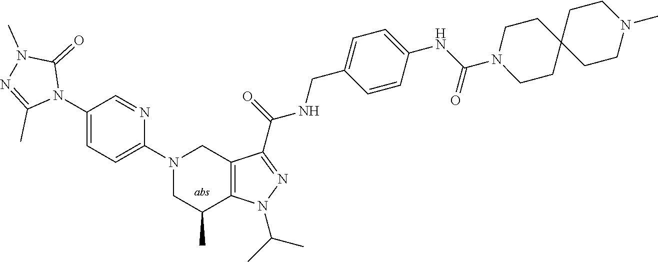 [SMILES: Cc1nn(C)c(=O)n1-c1ccc(N2Cc3c(C(=O)NCc4ccc(NC(=O)N5CCC6(CCN(C)CC6)CC5)cc4)nn(C(C)C)c3[C@@H](C)C2)nc1] | (S)-N-(4-((5-(5-(1,3-dimethyl-5-oxo- 1,5-dihydro-4H-1,2,4-triazol-4- yl)pyridin-2-yl)-1-isopropyl-7-methyl- 4,5,6,7-tetrahydro-1H-pyrazolo[4,3- c]pyridine-3- carboxamido)methyl)phenyl)-9- methyl-3,9-diazaspiro[5.5]undecane- 3-carboxamide |
| V84 | 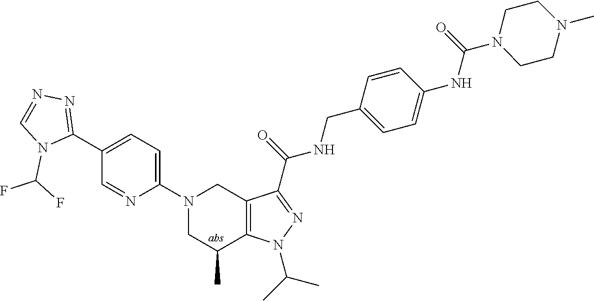 [SMILES: CC(C)n1nc(C(=O)NCc2ccc(NC(=O)N3CCN(C)CC3)cc2)c2c1[C@@H](C)CN(c1ccc(-c3nncn3C(F)F)cn1)C2] | (S)-5-(5-(4-(difluoromethyl)-4H-1,2,4- triazol-3-yl)pyridin-2-yl)-1-isopropyl- 7-methyl-N-(4-(4-methylpiperazine-1- carboxamido)benzyl)-4,5,6,7- tetrahydro-1H-pyrazolo[4,3- c]pyridine-3-carboxamide |
| V85 | 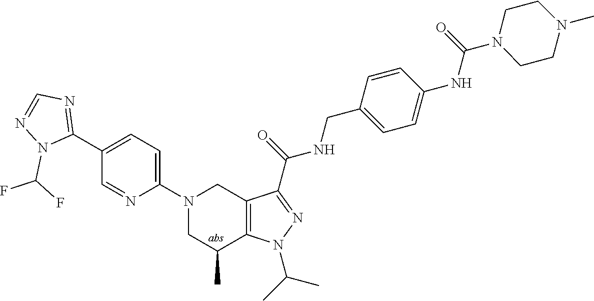 [SMILES: CC(C)n1nc(C(=O)NCc2ccc(NC(=O)N3CCN(C)CC3)cc2)c2c1[C@@H](C)CN(c1ccc(-c3ncnn3C(F)F)cn1)C2] | (S)-5-(5-(1-(difluoromethyl)-1H-1,2,4- triazol-5-yl)pyridin-2-yl)-1-isopropyl- 7-methyl-N-(4-(4-methylpiperazine-1- carboxamido)benzyl)-4,5,6,7- tetrahydro-1H-pyrazolo[4,3- c]pyridine-3-carboxamide |
| V86 | 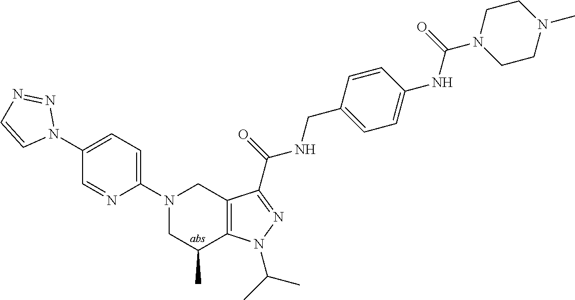 [SMILES: CC(C)n1nc(C(=O)NCc2ccc(NC(=O)N3CCN(C)CC3)cc2)c2c1[C@@H](C)CN(c1ccc(-n3ccnn3)cn1)C2] | (S)-5-(5-(1H-1,2,3-triazol-1-yl)pyridin- 2-yl)-1-isopropyl-7-methyl-N-(4-(4- methylpiperazine-1- carboxamido)benzyl)-4,5,6,7- tetrahydro-1H-pyrazolo[4,3- c]pyridine-3-carboxamide |
| V87 | 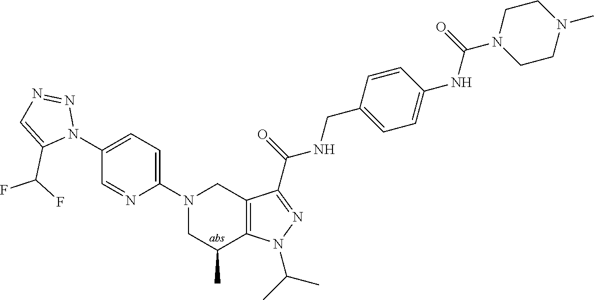 [SMILES: CC(C)n1nc(C(=O)NCc2ccc(NC(=O)N3CCN(C)CC3)cc2)c2c1[C@@H](C)CN(c1ccc(-n3nncc3C(F)F)cn1)C2] | (S)-5-(5-(5-(difluoromethyl)-1H-1,2,3- triazol-1-yl)pyridin-2-yl)-1-isopropyl- 7-methyl-N-(4-(4-methylpiperazine-1- carboxamido)benzyl)-4,5,6,7- tetrahydro-1H-pyrazolo[4,3- c]pyridine-3-carboxamide |
| V88 | 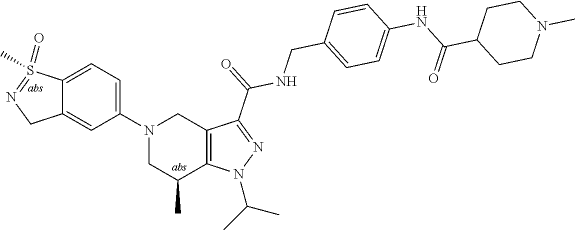 | (S)-1-isopropyl-7-methyl-5-((S)-1- methyl-1-oxido-3H-114- benzo[d]isothiazol-5-yl)-N-(4-(4- methylpiperazine-1- carboxamido)benzyl)-4,5,6,7- tetrahydro-1H-pyrazolo[4,3- c]pyridine-3-carboxamide |
| V89 | 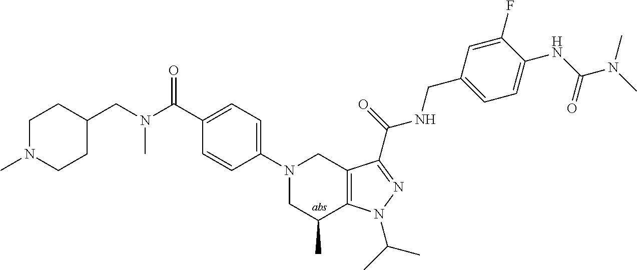 | (S)-N-(4-(3,3-dimethylureido)-3- fluorobenzyl)-1-isopropyl-7-methyl-5- (4-(methyl((1-methylpiperidin-4- yl)methyl)carbamoyl)phenyl)-4,5,6,7- tetrahydro-1H-pyrazolo[4,3- c]pyridine-3-carboxamide |
| V90 | 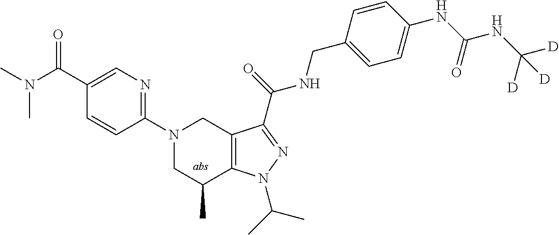 [SMILES: [2H]C([2H])([2H])NC(=O)Nc1ccc(CNC(=O)c2nn(C(C)C)c3c2CN(c2ccc(C(=O)N(C)C)cn2)C[C@@H]3C)cc1] | (S)-5-(5-(dimethylcarbamoyl)pyridin- 2-yl)-1-isopropyl-7-methyl-N-(4-(3- (methyl-d₃)ureido)benzyl)-4,5,6,7- tetrahydro-1H-pyrazolo[4,3- c]pyridine-3-carboxamide |
| V91 | 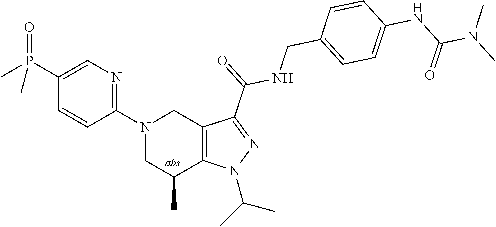 | (S)-5-(5-(dimethylphosphoryl)pyridin- 2-yl)-N-(4-(3,3- dimethylureido)benzyl)-1-isopropyl-7- methyl-4,5,6,7-tetrahydro-1H- pyrazolo[4,3-c]pyridine-3- carboxamide |
| V92 | 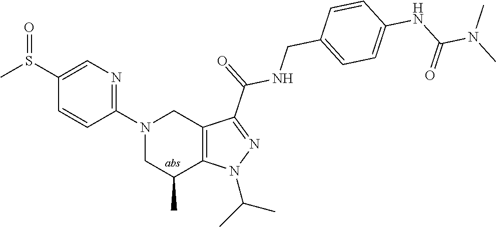 | (7S)-N-(4-(3,3- dimethylureido)benzyl)-1-isopropyl-7- methyl-5-(5-(methylsulfinyl)pyridin-2- yl)-4,5,6,7-tetrahydro-1H- pyrazolo[4,3-c]pyridine-3- carboxamide |
| V93 | 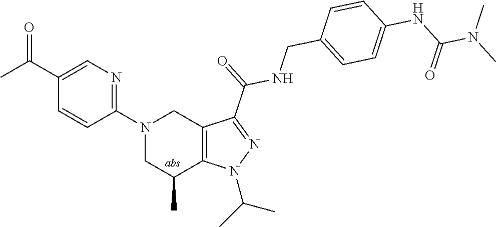 [SMILES: CC(=O)c1ccc(N2Cc3c(C(=O)NCc4ccc(NC(=O)N(C)C)cc4)nn(C(C)C)c3[C@@H](C)C2)nc1] | (S)-5-(5-acetylpyridin-2-yl)-N-(4-(3,3- dimethylureido)benzyl)-1-isopropyl-7- methyl-4,5,6,7-tetrahydro-1H- pyrazolo[4,3-c]pyridine-3- carboxamide |
| V94 | 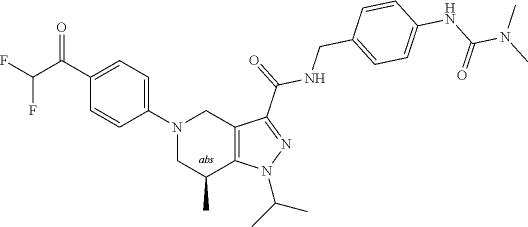 [SMILES: CC(C)n1nc(C(=O)NCc2ccc(NC(=O)N(C)C)cc2)c2c1[C@@H](C)CN(c1ccc(C(=O)C(F)F)cc1)C2] | (S)-5-(4-(2,2-difluoroacetyl)phenyl)- N-(4-(3,3-dimethylureido)benzyl)-1- isopropyl-7-methyl-4,5,6,7- tetrahydro-1H-pyrazolo[4,3- c]pyridine-3-carboxamide |
| V95 | 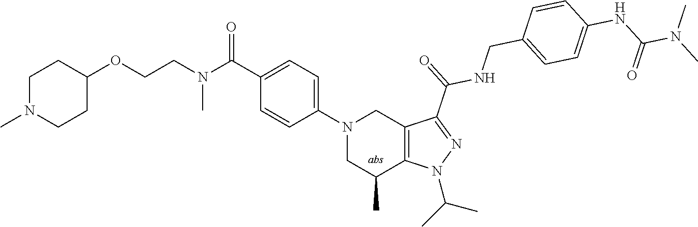 | (S)-N-(4-(3,3-dimethylureido)benzyl)- 1-isopropyl-7-methyl-5-(4-(methyl(2- ((1-methylpiperidin-4- yl)oxy)ethyl)carbamoyl)phenyl)- 4,5,6,7-tetrahydro-1H-pyrazolo[4,3- c]pyridine-3-carboxamide |
| V96 | 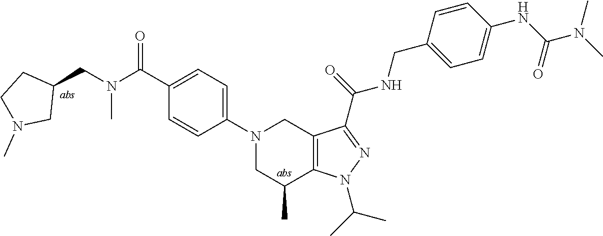 [SMILES: CC(C)n1nc(C(=O)NCc2ccc(NC(=O)N(C)C)cc2)c2c1[C@@H](C)CN(c1ccc(C(=O)N(C)C[C@@H]3CCN(C)C3)cc1)C2] | (S)-N-(4-(3,3-dimethylureido)benzyl)- 1-isopropyl-7-methyl-5-(4- (methyl(((R)-1-methylpyrrolidin-3- yl)methyl)carbamoyl)phenyl)-4,5,6,7- tetrahydro-1H-pyrazolo[4,3- c]pyridine-3-carboxamide |
| V97 | 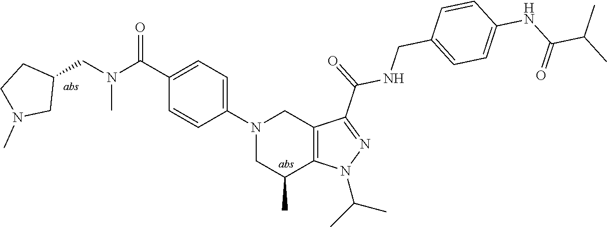 [SMILES: CC(C)C(=O)Nc1ccc(CNC(=O)c2nn(C(C)C)c3c2CN(c2ccc(C(=O)N(C)C[C@H]4CCN(C)C4)cc2)C[C@@H]3C)cc1] | (S)-N-(4-(3,3-dimethylureido)benzyl)- 1-isopropyl-7-methyl-5-(4- (methyl(((S)-1-methylpyrrolidin-3- yl)methyl)carbamoyl)phenyl)-4,5,6,7- tetrahydro-1H-pyrazolo[4,3- c]pyridine-3-carboxamide |
| V98 | 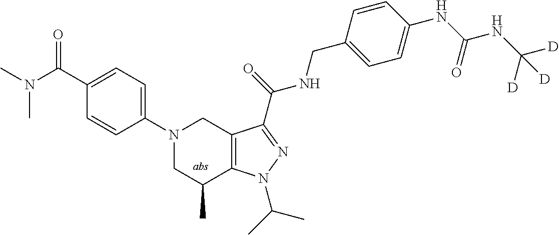 [SMILES: [2H]C([2H])([2H])NC(=O)Nc1ccc(CNC(=O)c2nn(C(C)C)c3c2CN(c2ccc(C(=O)N(C)C)cc2)C[C@@H]3C)cc1] | (S)-5-(4-(dimethylcarbamoyl)phenyl)- 1-isopropyl-7-methyl-N-(4-(3-(methyl- d₃)ureido)benzyl)-4,5,6,7-tetrahydro- 1H-pyrazolo[4,3-c]pyridine-3- carboxamide |
| V99 | 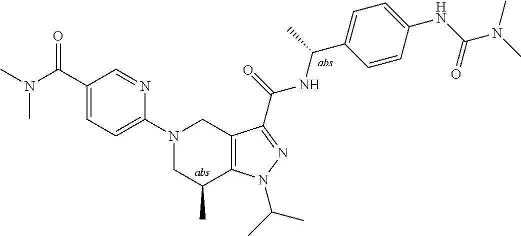 | (S)-5-(5-(dimethylcarbamoyl)pyridin- 2-yl)-N-((R)-1-(4-(3,3- dimethylureido)phenyl)ethyl)-1- isopropyl-7-methyl-4,5,6,7- tetrahydro-1H-pyrazolo[4,3- c]pyridine-3-carboxamide |
| V100 | 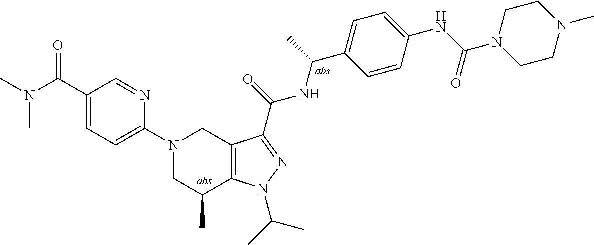 | (S)-5-(5-(dimethylcarbamoyl)pyridin- 2-yl)-1-isopropyl-7-methyl-N-((R)-1- (4-(4-methylpiperazine-1- carboxamido)phenyl)ethyl)-4,5,6,7- tetrahydro-1H-pyrazolo[4,3- c]pyridine-3-carboxamide |
| V101 | 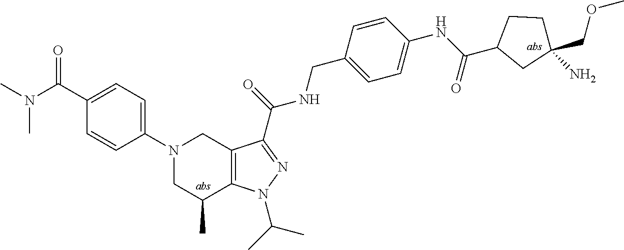 | (S)-N-(4-((S)-3-amino-3- (methoxymethyl)pyrrolidine-1- carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V102 | 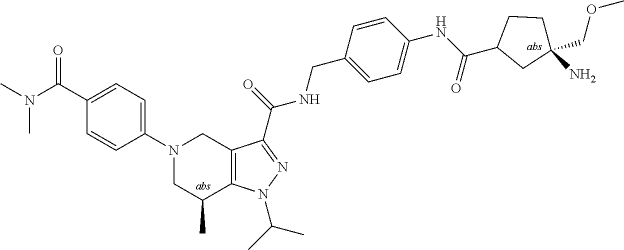 | (S)-N-(4-((R)-3-amino-3- (methoxymethyl)pyrrolidine-1- carboxamido)benzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V103 | 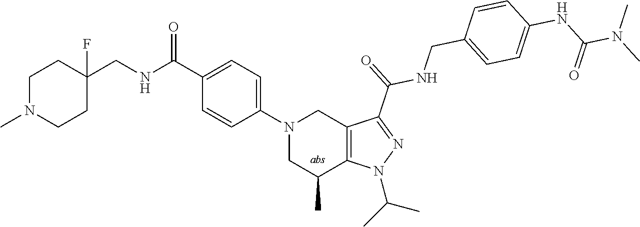 | (S)-N-(4-(3,3-dimethylureido)benzyl)- 5-(4-(((4-fluoro-1-methylpiperidin-4- yl)methyl)(methyl)carbamoyl)phenyl)- 1-isopropyl-7-methyl-4,5,6,7- tetrahydro-1H-pyrazolo[4,3- c]pyridine-3-carboxamide |
| V104 | 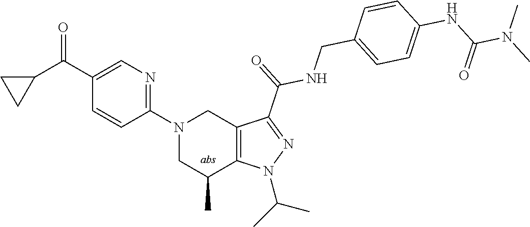 [SMILES: CC(C)n1nc(C(=O)NCc2ccc(NC(=O)N(C)C)cc2)c2c1[C@@H](C)CN(c1ccc(C(=O)C3CC3)cn1)C2] | (S)-5-(5- (cyclopropanecarbonyl)pyridin-2-yl)- N-(4-(3,3-dimethylureido)benzyl)-1- isopropyl-7-methyl-4,5,6,7- tetrahydro-1H-pyrazolo[4,3- c]pyridine-3-carboxamide |
| V105 | 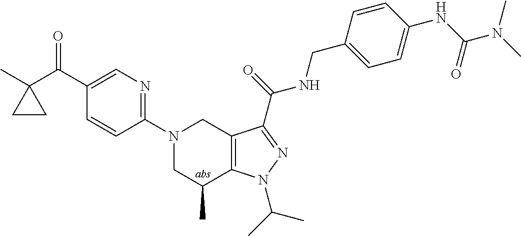 | (S)-N-(4-(3,3-dimethylureido)benzyl)- 1-isopropyl-7-methyl-5-(5-(1- methylcyclopropane-1- carbonyl)pyridin-2-yl)-4,5,6,7- tetrahydro-1H-pyrazolo[4,3- c]pyridine-3-carboxamide |
| V106 | 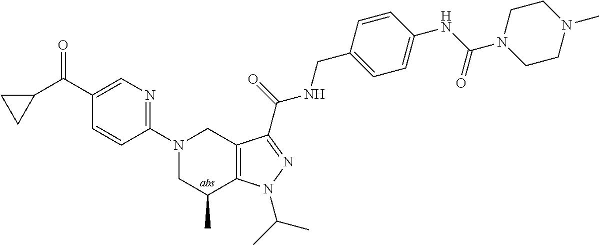 [SMILES: CC(C)n1nc(C(=O)NCc2ccc(NC(=O)N3CCN(C)CC3)cc2)c2c1[C@@H](C)CN(c1ccc(C(=O)C3CC3)cn1)C2] | (S)-5-(5- (cyclopropanecarbonyl)pyridin-2-yl)- 1-isopropyl-7-methyl-N-(4-(4- methylpiperazine-1- carboxamido)benzyl)-4,5,6,7- tetrahydro-1H-pyrazolo[4,3- c]pyridine-3-carboxamide |
| V107 | 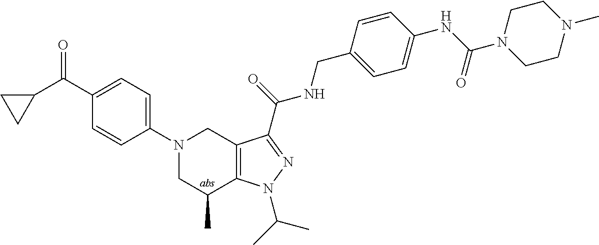 [SMILES: CC(C)n1nc(C(=O)NCc2ccc(NC(=O)N3CCN(C)CC3)cc2)c2c1[C@@H](C)CN(c1ccc(C(=O)C3CC3)cc1)C2] | (S)-5-(4- (cyclopropanecarbonyl)phenyl)-1- isopropyl-7-methyl-N-(4-(4- methylpiperazine-1- carboxamido)benzyl)-4,5,6,7- tetrahydro-1H-pyrazolo[4,3- c]pyridine-3-carboxamide |
| V108 | 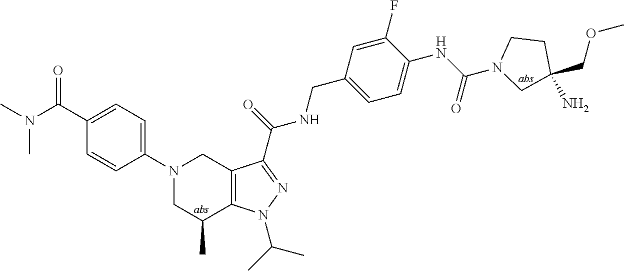 | (S)-N-(4-((S)-3-amino-3- (methoxymethyl)pyrrolidine-1- carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V109 | 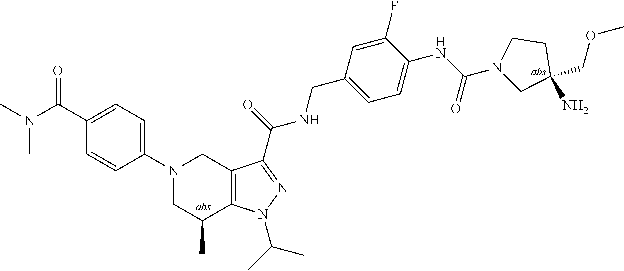 | (S)-N-(4-((R)-3-amino-3- (methoxymethyl)pyrrolidine-1- carboxamido)-3-fluorobenzyl)-5-(4- (dimethylcarbamoyl)phenyl)-1- isopropyl-7-methyl-4,5,6,7- tetrahydro-1H-pyrazolo[4,3- c]pyridine-3-carboxamide |
| V110 | 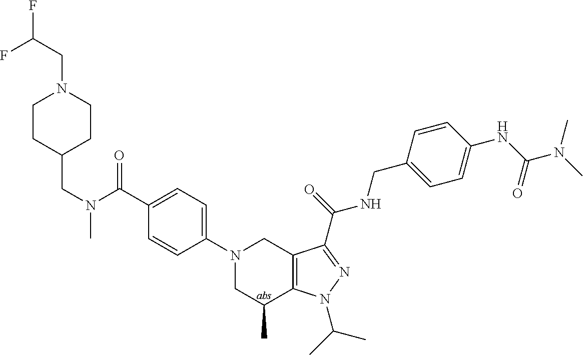 [SMILES: CC(C)n1nc(C(=O)NCc2ccc(NC(=O)N(C)C)cc2)c2c1[C@@H](C)CN(c1ccc(C(=O)N(C)CC3CCN(CC(F)F)CC3)cc1)C2] | (S)-5-(4-(((1-(2,2- difluoroethyl)piperidin-4- yl)methyl)(methyl)carbamoyl)phenyl)- N-(4-(3,3-dimethylureido)benzyl)-1- isopropyl-7-methyl-4,5,6,7- tetrahydro-1H-pyrazolo[4,3- c]pyridine-3-carboxamide |
| V111 | 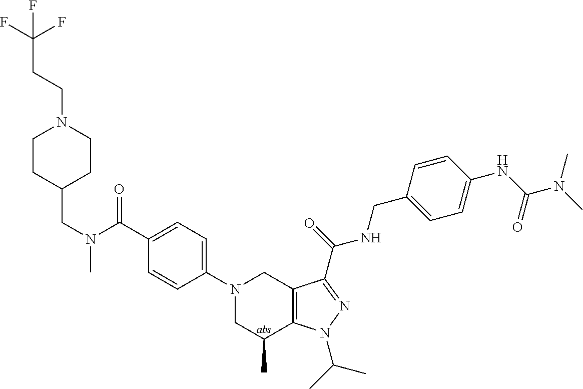 [SMILES: CC(C)n1nc(C(=O)NCc2ccc(NC(=O)N(C)C)cc2)c2c1[C@@H](C)CN(c1ccc(C(=O)N(C)CC3CCN(CCC(F)(F)F)CC3)cc1)C2] | (S)-N-(4-(3,3-dimethylureido)benzyl)- 1-isopropyl-7-methyl-5-(4-(methyl((1- (3,3,3-trifluoropropyl)piperidin-4- yl)methyl)carbamoyl)phenyl)-4,5,6,7- tetrahydro-1H-pyrazolo[4,3- c]pyridine-3-carboxamide |
| V112 | 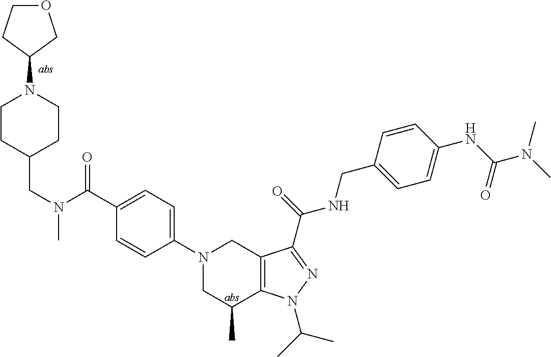 [SMILES: CC(C)n1nc(C(=O)NCc2ccc(NC(=O)N(C)C)cc2)c2c1[C@@H](C)CN(c1ccc(C(=O)N(C)CC3CCN([C@H]4CCOC4)CC3)cc1)C2] | (S)-N-(4-(3,3-dimethylureido)benzyl)- 1-isopropyl-7-methyl-5-(4-(methyl((1- ((S)-tetrahydrofuran-3-yl)piperidin-4- yl)methyl)carbamoyl)phenyl)-4,5,6,7- tetrahydro-1H-pyrazolo[4,3- c]pyridine-3-carboxamide |
| V113 | 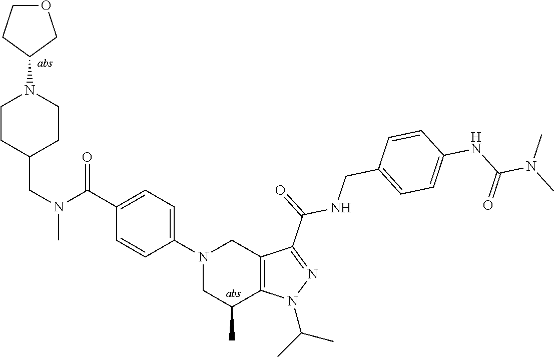 [SMILES: CC(C)n1nc(C(=O)NCc2ccc(NC(=O)N(C)C)cc2)c2c1[C@@H](C)CN(c1ccc(C(=O)N(C)CC3CCN([C@@H]4CCOC4)CC3)cc1)C2] | (S)-N-(4-(3,3-dimethylureido)benzyl)- 1-isopropyl-7-methyl-5-(4-(methyl((1- ((R)-tetrahydrofuran-3-yl)piperidin-4- yl)methyl)carbamoyl)phenyl)-4,5,6,7- tetrahydro-1H-pyrazolo[4,3- c]pyridine-3-carboxamide |
| V114 | 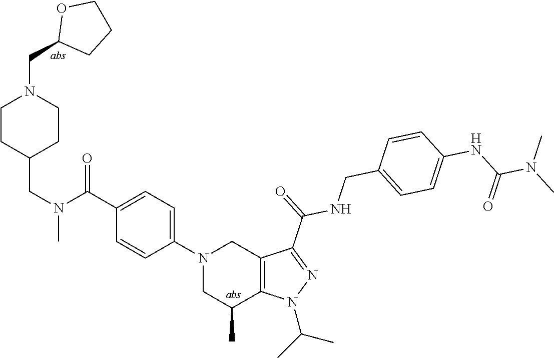 [SMILES: CC(C)n1nc(C(=O)NCc2ccc(NC(=O)N(C)C)cc2)c2c1[C@@H](C)CN(c1ccc(C(=O)N(C)CC3CCN(C[C@@H]4CCCO4)CC3)cc1)C2] | (S)-N-(4-(3,3-dimethylureido)benzyl)- 1-isopropyl-7-methyl-5-(4-(methyl((1- (((S)-tetrahydrofuran-2- yl)methyl)piperidin-4- yl)methyl)carbamoyl)phenyl)-4,5,6,7- tetrahydro-1H-pyrazolo[4,3- c]pyridine-3-carboxamide |
| V115 | 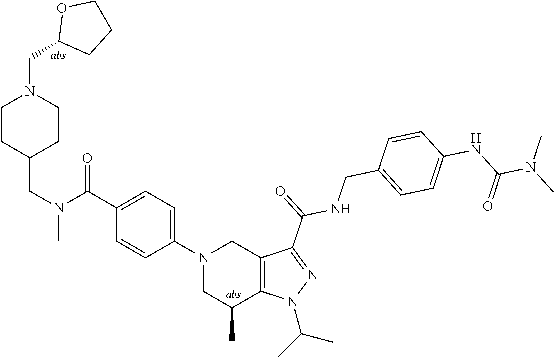 [SMILES: CC(C)n1nc(C(=O)NCc2ccc(NC(=O)N(C)C)cc2)c2c1[C@@H](C)CN(c1ccc(C(=O)N(C)CC3CCN(C[C@H]4CCCO4)CC3)cc1)C2] | (S)-N-(4-(3,3-dimethylureido)benzyl)- 1-isopropyl-7-methyl-5-(4-(methyl((1- (((R)-tetrahydrofuran-2- yl)methyl)piperidin-4- yl)methyl)carbamoyl)phenyl)-4,5,6,7- tetrahydro-1H-pyrazolo[4,3- c]pyridine-3-carboxamide |
| V116 | 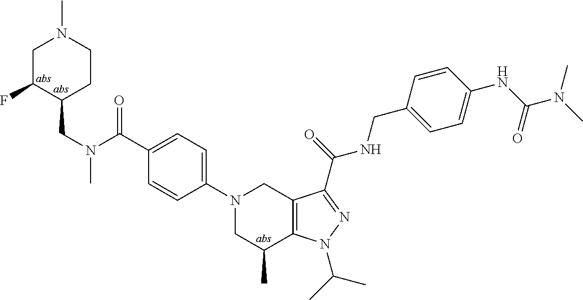 [SMILES: CC(C)n1nc(C(=O)NCc2ccc(NC(=O)N(C)C)cc2)c2c1[C@@H](C)CN(c1ccc(C(=O)N(C)C[C@@H]3CCN(C)C[C@@H]3F)cc1)C2] | (S)-N-(4-(3,3-dimethylureido)benzyl)- 5-(4-((((3R,4S)-3-fluoro-1- methylpiperidin-4- yl)methyl)(methyl)carbamoyl)phenyl)- 1-isopropyl-7-methyl-4,5,6,7- tetrahydro-1H-pyrazolo[4,3- c]pyridine-3-carboxamide |
| V117 | 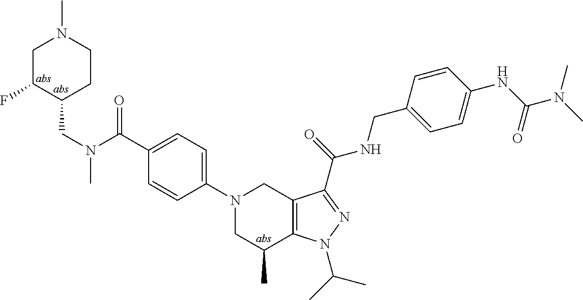 [SMILES: CC(C)n1nc(C(=O)NCc2ccc(NC(=O)N(C)C)cc2)c2c1[C@@H](C)CN(c1ccc(C(=O)N(C)C[C@H]3CCN(C)C[C@H]3F)cc1)C2] | (S)-N-(4-(3,3-dimethylureido)benzyl)- 5-(4-((((3S,4R)-3-fluoro-1- methylpiperidin-4- yl)methyl)(methyl)carbamoyl)phenyl)- 1-isopropyl-7-methyl-4,5,6,7- tetrahydro-1H-pyrazolo[4,3- c]pyridine-3-carboxamide |
| V118 | 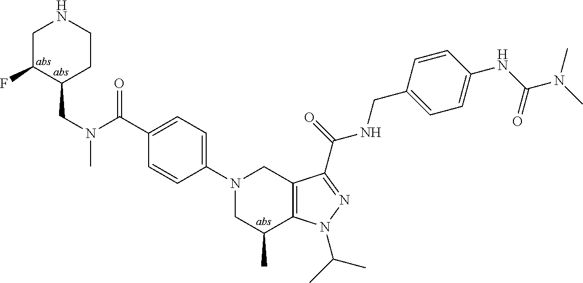 [SMILES: CC(C)n1nc(C(=O)NCc2ccc(NC(=O)N(C)C)cc2)c2c1[C@@H](C)CN(c1ccc(C(=O)N(C)C[C@@H]3CCNC[C@@H]3F)cc1)C2] | (S)-N-(4-(3,3-dimethylureido)benzyl)- 5-(4-((((3R,4S)-3-fluoropiperidin-4- yl)methyl)(methyl)carbamoyl)phenyl)- 1-isopropyl-7-methyl-4,5,6,7- tetrahydro-1H-pyrazolo[4,3- c]pyridine-3-carboxamide |
| V119 | 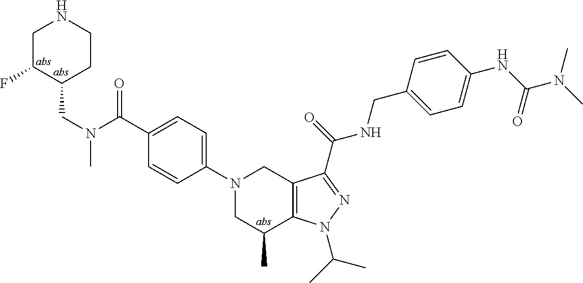 [SMILES: CC(C)n1nc(C(=O)NCc2ccc(NC(=O)N(C)C)cc2)c2c1[C@@H](C)CN(c1ccc(C(=O)N(C)C[C@H]3CCNC[C@H]3F)cc1)C2] | (S)-N-(4-(3,3-dimethylureido)benzyl)- 5-(4-((((3S,4R)-3-fluoropiperidin-4- yl)methyl)(methyl)carbamoyl)phenyl)- 1-isopropyl-7-methyl-4,5,6,7- tetrahydro-1H-pyrazolo[4,3- c]pyridine-3-carboxamide |
| V120 | 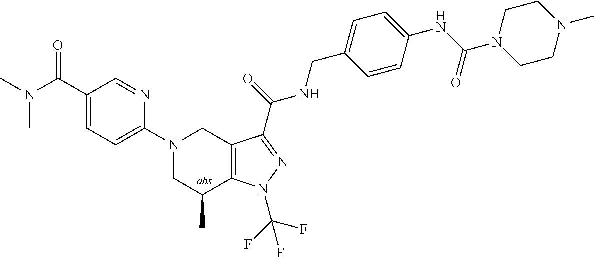 | (S)-5-(5-(dimethylcarbamoyl)pyridin- 2-yl)-7-methyl-N-(4-(4- methylpiperazine-1- carboxamido)benzyl)-1- (trifluoromethyl)-4,5,6,7-tetrahydro- 1H-pyrazolo[4,3-c]pyridine-3- carboxamide |
| V121 | 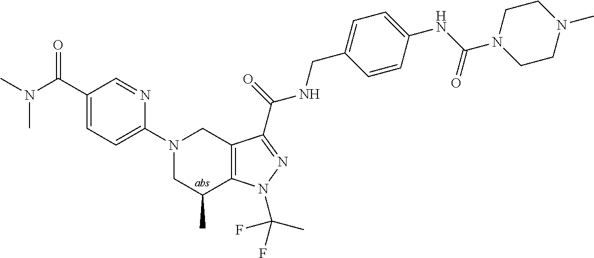 | (S)-1-(1,1-difluoroethyl)-5-(5- (dimethylcarbamoyl)pyridin-2-yl)-7- methyl-N-(4-(4-methylpiperazine-1- carboxamido)benzyl)-4,5,6,7- tetrahydro-1H-pyrazolo[4,3- c]pyridine-3-carboxamide |
| V122 | 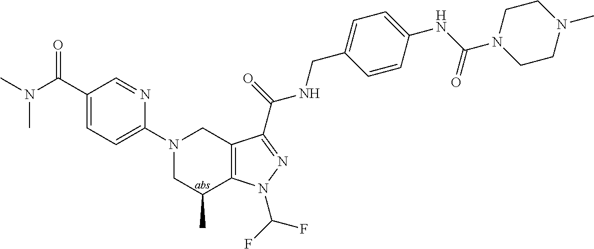 | (S)-1-(difluoromethyl)-5-(5- (dimethylcarbamoyl)pyridin-2-yl)-7- methyl-N-(4-(4-methylpiperazine-1- carboxamido)benzyl)-4,5,6,7- tetrahydro-1H-pyrazolo[4,3- c]pyridine-3-carboxamide |
| V123 | 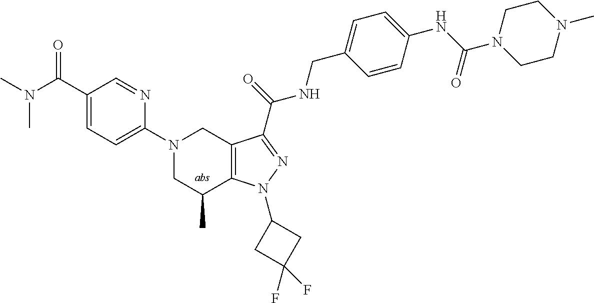 | (S)-1-(3,3-difluorocyclobutyl)-5-(5- (dimethylcarbamoyl)pyridin-2-yl)-7- methyl-N-(4-(4-methylpiperazine-1- carboxamido)benzyl)-4,5,6,7- tetrahydro-1H-pyrazolo[4,3- c]pyridine-3-carboxamide |
| V124 | 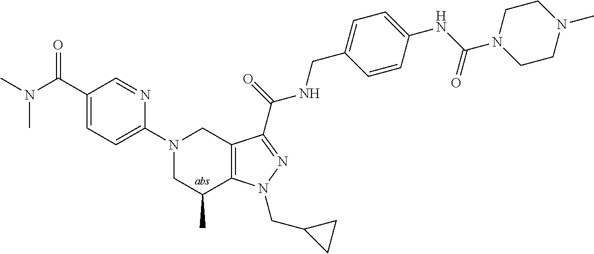 | (S)-1-(cyclopropylmethyl)-5-(5- (dimethylcarbamoyl)pyridin-2-yl)-7- methyl-N-(4-(4-methylpiperazine-1- carboxamido)benzyl)-4,5,6,7- tetrahydro-1H-pyrazolo[4,3- c]pyridine-3-carboxamide |
| V125 | 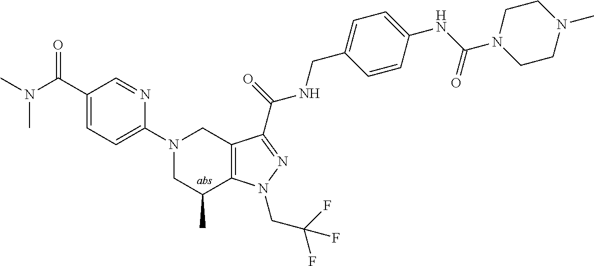 | (S)-5-(5-(dimethylcarbamoyl)pyridin- 2-yl)-7-methyl-N-(4-(4- methylpiperazine-1- carboxamido)benzyl)-1-(2,2,2- trifluoroethyl)-4,5,6,7-tetrahydro-1H- pyrazolo[4,3-c]pyridine-3- carboxamide |
| V126 | 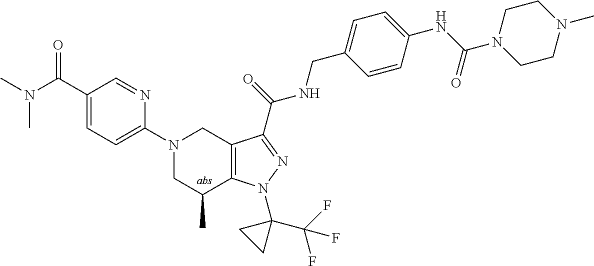 | (S)-5-(5-(dimethylcarbamoyl)pyridin- 2-yl)-7-methyl-N-(4-(4- methylpiperazine-1- carboxamido)benzyl)-1-(1- (trifluoromethyl)cyclopropyl)-4,5,6,7- tetrahydro-1H-pyrazolo[4,3- c]pyridine-3-carboxamide |
| V127 | 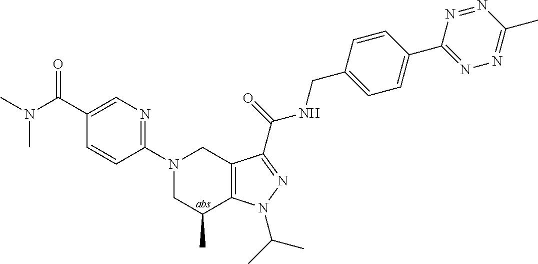 | (S)-5-(5-(dimethylcarbamoyl)pyridin- 2-yl)-1-isopropyl-7-methyl-N-(4-(6- methyl-1,2,4,5-tetrazin-3-yl)benzyl)- 4,5,6,7-tetrahydro-1H-pyrazolo[4,3- c]pyridine-3-carboxamide |
| V128 | 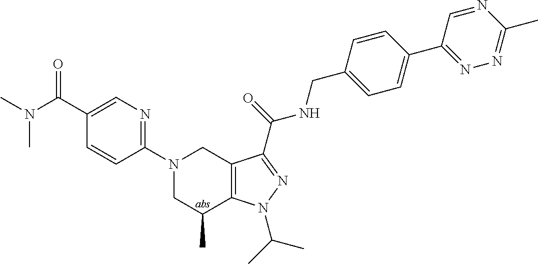 | (S)-5-(5-(dimethylcarbamoyl)pyridin- 2-yl)-1-isopropyl-7-methyl-N-(4-(3- methyl-1,2,4-triazin-6-yl)benzyl)- 4,5,6,7-tetrahydro-1H-pyrazolo[4,3- c]pyridine-3-carboxamide |
| V129 | 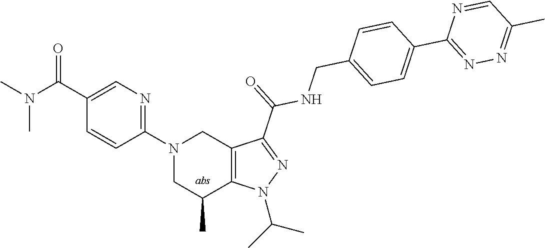 | (S)-5-(5-(dimethylcarbamoyl)pyridin- 2-yl)-1-isopropyl-7-methyl-N-(4-(6- methyl-1,2,4-triazin-3-yl)benzyl)- 4,5,6,7-tetrahydro-1H-pyrazolo[4,3- c]pyridine-3-carboxamide |
| V130 | 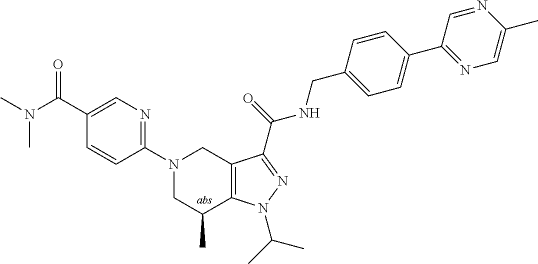 | (S)-5-(5-(dimethylcarbamoyl)pyridin- 2-yl)-1-isopropyl-7-methyl-N-(4-(5- methylpyrazin-2-yl)benzyl)-4,5,6,7- tetrahydro-1H-pyrazolo[4,3- c]pyridine-3-carboxamide |
| V131 | 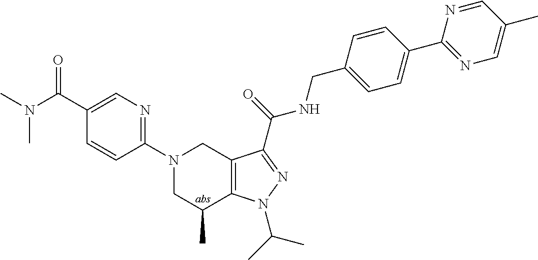 | (S)-5-(5-(dimethylcarbamoyl)pyridin- 2-yl)-1-isopropyl-7-methyl-N-(4-(5- methylpyrimidin-2-yl)benzyl)-4,5,6,7- tetrahydro-1H-pyrazolo[4,3- c]pyridine-3-carboxamide |
| V132 | 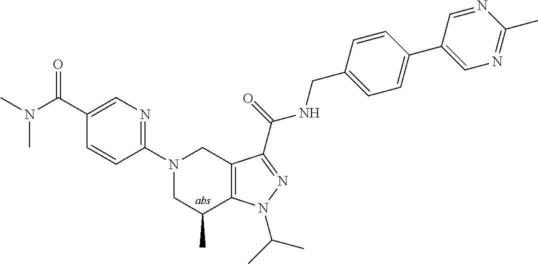 | (S)-5-(5-(dimethylcarbamoyl)pyridin- 2-yl)-1-isopropyl-7-methyl-N-(4-(2- methylpyrimidin-5-yl)benzyl)-4,5,6,7- tetrahydro-1H-pyrazolo[4,3- c]pyridine-3-carboxamide |
| V133 | 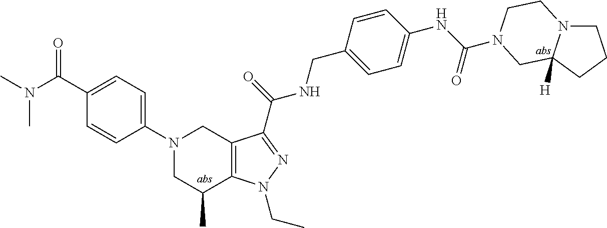 [SMILES: CCn1nc(C(=O)NCc2ccc(NC(=O)N3CCN4CCC[C@@H]4C3)cc2)c2c1[C@@H](C)CN(c1ccc(C(=O)N(C)C)cc1)C2] | (S)-5-(4-(dimethylcarbamoyl)phenyl)- 1-ethyl-7-methyl-N-(4-((R)- octahydropyrrolo[1,2-a]pyrazine-2- carboxamido)benzyl)-4,5,6,7- tetrahydro-1H-pyrazolo[4,3- c]pyridine-3-carboxamide |
| V134 | 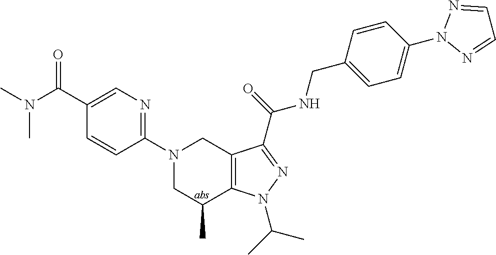 [SMILES: CC(C)n1nc(C(=O)NCc2ccc(-n3nccn3)cc2)c2c1[C@@H](C)CN(c1ccc(C(=O)N(C)C)cn1)C2] | (S)-N-(4-(2H-1,2,3-triazol-2- yl)benzyl)-5-(5- (dimethylcarbamoyl)pyridin-2-yl)-1- isopropyl-7-methyl-4,5,6,7- tetrahydro-1H-pyrazolo[4,3- c]pyridine-3-carboxamide |
| V135 | 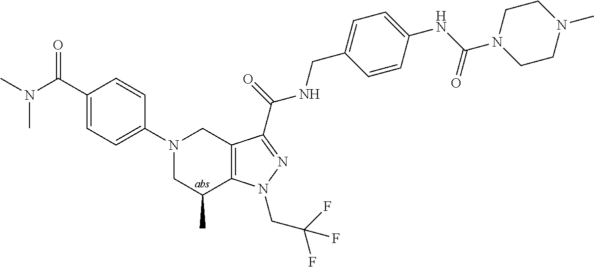 | (S)-5-(4-(dimethylcarbamoyl)phenyl)- 7-methyl-N-(4-(4-methylpiperazine-1- carboxamido)benzyl)-1-(2,2,2- trifluoroethyl)-4,5,6,7-tetrahydro-1H- pyrazolo[4,3-c]pyridine-3- carboxamide |
| V136 | 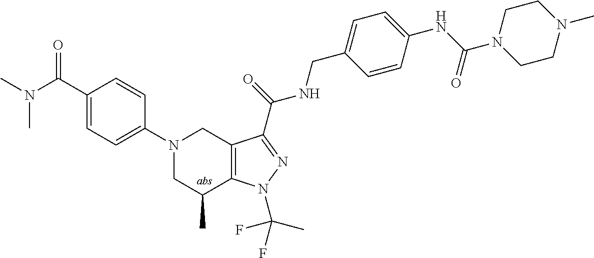 | (S)-1-(1,1-difluoroethyl)-5-(4- (dimethylcarbamoyl)phenyl)-7- methyl-N-(4-(4-methylpiperazine-1- carboxamido)benzyl)-4,5,6,7- tetrahydro-1H-pyrazolo[4,3- c]pyridine-3-carboxamide |
| V137 | 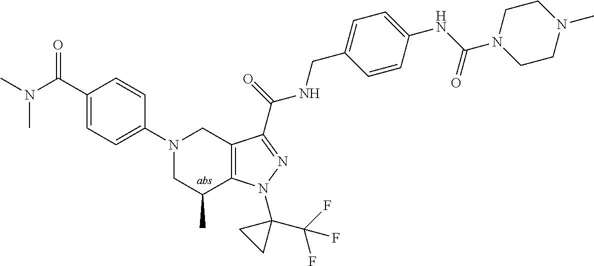 | (S)-5-(4-(dimethylcarbamoyl)phenyl)- 7-methyl-N-(4-(4-methylpiperazine-1- carboxamido)benzyl)-1-(1- (trifluoromethyl)cyclopropyl)-4,5,6,7- tetrahydro-1H-pyrazolo[4,3- c]pyridine-3-carboxamide |
| V138 | 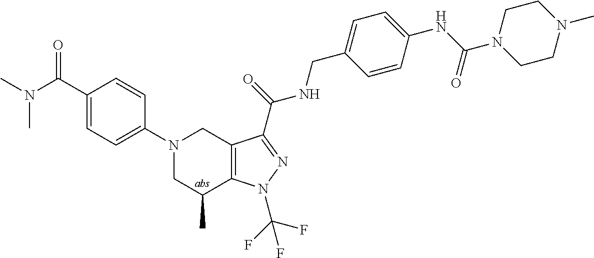 | (S)-5-(4-(dimethylcarbamoyl)phenyl)- 7-methyl-N-(4-(4-methylpiperazine-1- carboxamido)benzyl)-1- (trifluoromethyl)-4,5,6,7-tetrahydro- 1H-pyrazolo[4,3-c]pyridine-3- carboxamide |
| V139 | 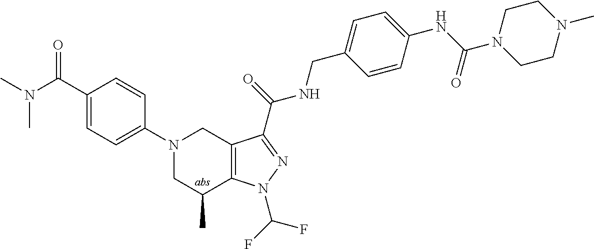 | (S)-1-(difluoromethyl)-5-(4- (dimethylcarbamoyl)phenyl)-7- methyl-N-(4-(4-methylpiperazine-1- carboxamido)benzyl)-4,5,6,7- tetrahydro-1H-pyrazolo[4,3- c]pyridine-3-carboxamide |
| V140 | 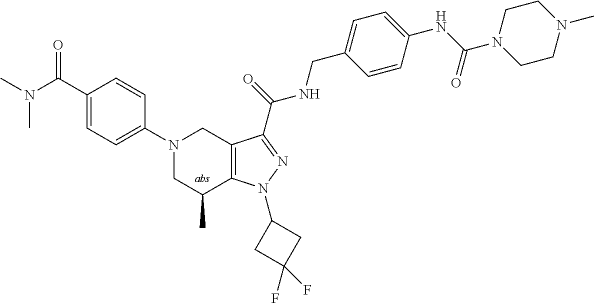 | (S)-1-(3,3-difluorocyclobutyl)-5-(4- (dimethylcarbamoyl)phenyl)-7- methyl-N-(4-(4-methylpiperazine-1- carboxamido)benzyl)-4,5,6,7- tetrahydro-1H-pyrazolo[4,3- c]pyridine-3-carboxamide |
| V141 | 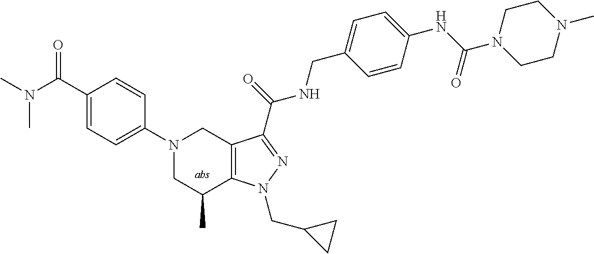 | (S)-1-(cyclopropylmethyl)-5-(4- (dimethylcarbamoyl)phenyl)-7- methyl-N-(4-(4-methylpiperazine-1- carboxamido)benzyl)-4,5,6,7- tetrahydro-1H-pyrazolo[4,3- c]pyridine-3-carboxamide |
| V142 | 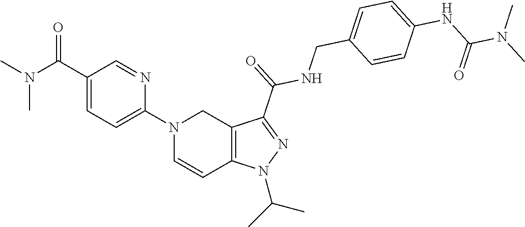 | (5-(5-(dimethylcarbamoyl)pyridin-2- yl)-N-(4-(3,3-dimethylureido)benzyl)- 1-isopropyl-4,5-dihydro-1H- pyrazolo[4,3-c]pyridine-3- carboxamide |

TABLE 2A

| Example | | |
|---|---|---|
| # | Structure | Chem Draw Name |
| V1A | 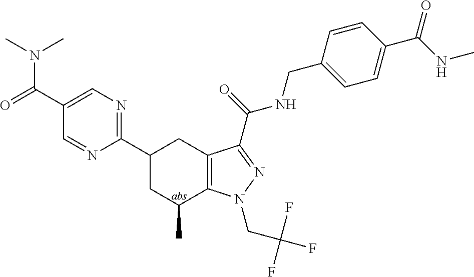 [SMILES: CNC(=O)c1ccc(CNC(=O)c2nn(CC(F)(F)F)c3c2CC(c2ncc(C(=O)N(C)C)cn2)C[C@@H]3C)cc1] | (7S)-5-(5- (dimethylcarbamoyl)pyrimidin-2- yl)-7-methyl-N-(4- (methylcarbamoyl)benzyl)-1- (2,2,2-trifluoroethyl)-4,5,6,7- tetrahydro-1H-indazole-3- carboxamide |
| V2A | 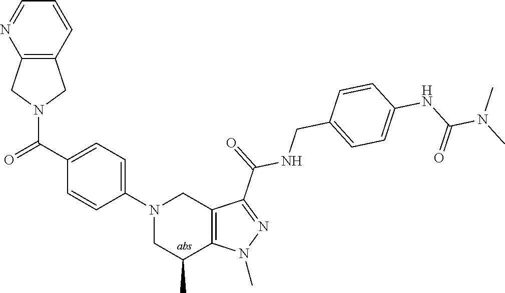 [SMILES: C[C@H]1CN(c2ccc(C(=O)N3Cc4cccnc4C3)cc2)Cc2c(C(=O)NCc3ccc(NC(=O)N(C)C)cc3)nn(C)c21] | (S)-5-(4-(6,7-dihydro-5H- pyrrolo[3,4-b]pyridine-6- carbonyl)phenyl)-N-(4-(3,3- dimethylureido)benzyl)-1,7- dimethyl-4,5,6,7-tetrahydro-1H- pyrazolo[4,3-c]pyridine-3- carboxamide |
| V3A | 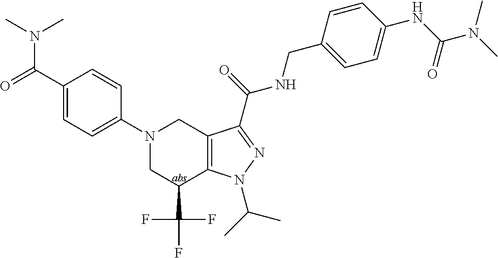 [SMILES: CC(C)n1nc(C(=O)NCc2ccc(NC(=O)N(C)C)cc2)c2c1[C@@H](C(F)(F)F)CN(c1ccc(C(=O)N(C)C)cc1)C2] | (S)-5-(4- (dimethylcarbamoyl)phenyl)-N-(4- (3,3-dimethylureido)benzyl)-1- isopropyl-7-(trifluoromethyl)- 4,5,6,7-tetrahydro-1H- pyrazolo[4,3-c]pyridine-3- carboxamide |
| V4A | 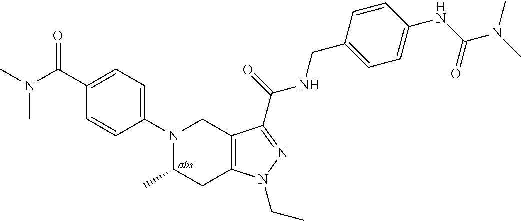 | (S)-5-(4- (dimethylcarbamoyl)phenyl)-N-(4- (3,3-dimethylureido)benzyl)-1- ethyl-6-methyl-4,5,6,7-tetrahydro- 1H-pyrazolo[4,3-c]pyridine-3- carboxamide |
| V5A | 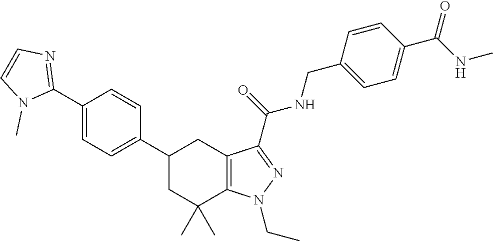 [SMILES: CCn1nc(C(=O)NCc2ccc(C(=O)NC)cc2)c2c1C(C)(C)CC(c1ccc(-c3nccn3C)cc1)C2] | 1-ethyl-7,7-dimethyl-5-(4-(1- methyl-1H-imidazol-2-yl)phenyl)- N-(4-(methylcarbamoyl)benzyl)- 4,5,6,7-tetrahydro-1H-indazole-3- carboxamide |
| V6A | 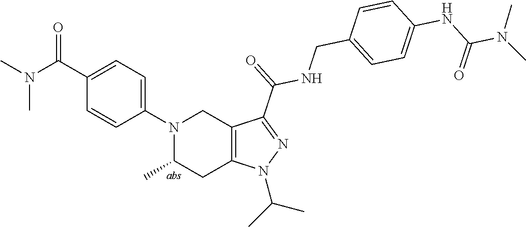 | (S)-5-(4- (dimethylcarbamoyl)phenyl)-N-(4- (3,3-dimethylureido)benzyl)-1- isopropyl-6-methyl- 4,5,6,7-tetrahydro- 1H-pyrazolo[4,3-c]pyridine-3- carboxamide |
| V7A | 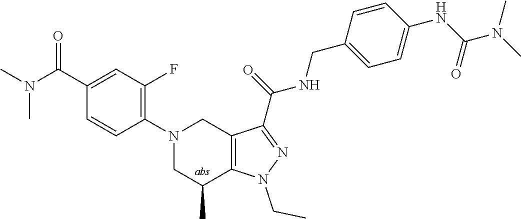 | (S)-5-(4-(dimethylcarbamoyl)-2- fluorophenyl)-N-(4-(3,3- dimethylureido)benzyl)-1-ethyl-7- methyl-4,5,6,7-tetrahydro- 1H-pyrazolo[4,3-c]pyridine-3- carboxamide |
| V8A | 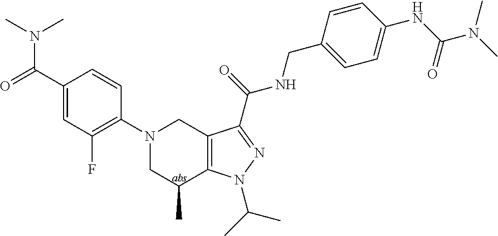 [SMILES: CC(C)n1nc(C(=O)NCc2ccc(NC(=O)N(C)C)cc2)c2c1[C@@H](C)CN(c1ccc(C(=O)N(C)C)cc1F)C2] | (S)-5-(4-(dimethylcarbamoyl)-2- fluorophenyl)-N-(4-(3,3- dimethylureido)benzyl)-1- isopropyl-7-methyl-4,5,6,7- tetrahydro-1H-pyrazolo[4,3-c] pyridine-3-carboxamide |
| V9A | 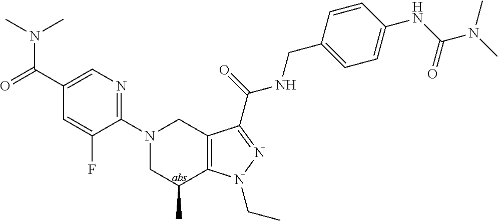 [SMILES: CCn1nc(C(=O)NCc2ccc(NC(=O)N(C)C)cc2)c2c1[C@@H](C)CN(c1ncc(C(=O)N(C)C)cc1F)C2] | (S)-5-(5-(dimethylcarbamoyl)-3- fluoropyridin-2-yl)-N-(4-(3,3- dimethylureido)benzyl)-1-ethyl-7- methyl-4,5,6,7-tetrahydro- 1H-pyrazolo[4,3-c]pyridine-3- carboxamide |
| V10A | 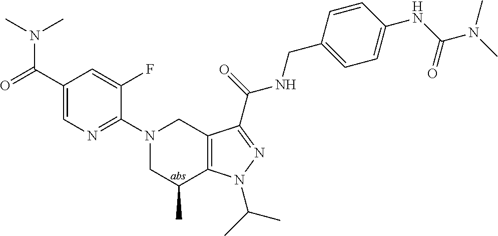 [SMILES: CC(C)n1nc(C(=O)NCc2ccc(NC(=O)N(C)C)cc2)c2c1[C@@H](C)CN(c1ncc(C(=O)N(C)C)cc1F)C2] | (S)-5-(5-(dimethylcarbamoyl)-3- fluoropyridin-2-yl)-N-(4-(3,3- dimethylureido)benzyl)-1- isopropyl-7-methyl- 4,5,6,7-tetrahydro- 1H-pyrazolo[4,3-c]pyridine-3- carboxamide |
| V11A | 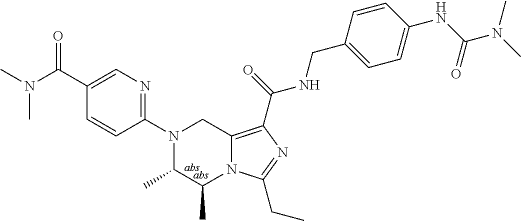 | (5S,6S)-7-(5- (dimethylcarbamoyl)pyridin-2-yl)- N-(4-(3,3-dimethylureido)benzyl)- 3-ethyl-5,6-dimethyl-5,6,7,8- tetrahydroimidazo[1,5-a]pyrazine- 1-carboxamide |
| V12A | 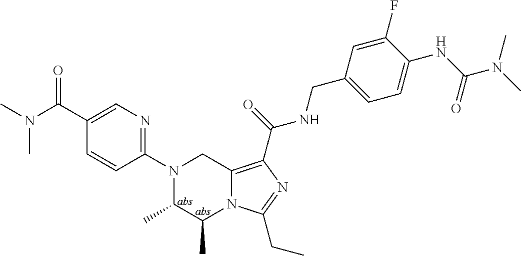 | (5S,6S)-7-(5- (dimethylcarbamoyl)pyridin-2-yl)- N-(4-(3,3-dimethylureido)-3- fluorobenzyl)-3-ethyl-5,6-dimethyl- 5,6,7,8-tetrahydroimidazo[1,5-a] pyrazine-1-carboxamide |
| V13A | 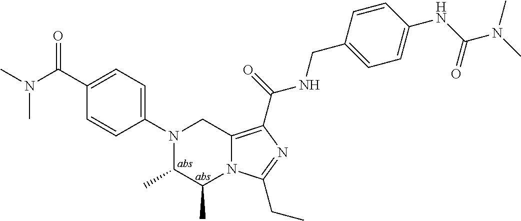 | (5S,6S)-7-(4- (dimethylcarbamoyl)phenyl)-N-(4- (3,3-dimethylureido)benzyl)-3- ethyl-5,6-dimethyl- 5,6,7,8-tetrahydroimidazo[1,5-a] pyrazine-1-carboxamide |
| V14A | 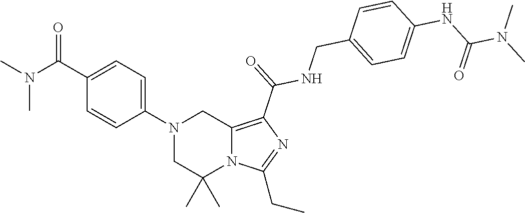 | 7-(4-(dimethylcarbamoyl)phenyl)- N-(4-(3,3-dimethylureido)benzyl)- 3-ethyl-5,5-dimethyl- 5,6,7,8-tetrahydroimidazo[1,5-a] pyrazine-1-carboxamide |
| V15A | 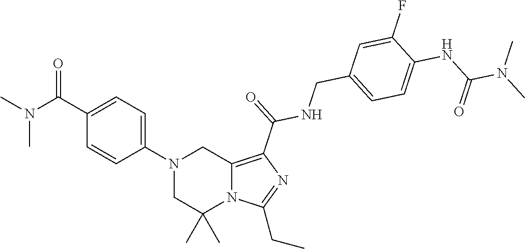 | 7-(4-(dimethylcarbamoyl)phenyl)- N-(4-(3,3-dimethylureido)-3- fluorobenzyl)-3-ethyl-5,5-dimethyl- 5,6,7,8-tetrahydroimidazo[1,5-a] pyrazine-1-carboxamide |
| V16A | 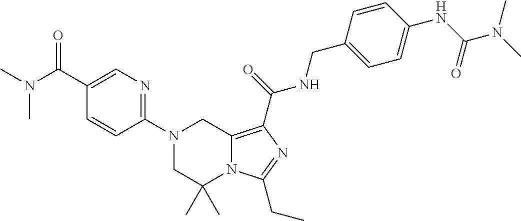 | 7-(5-(dimethylcarbamoyl)pyridin- 2-yl)-N-(4-(3,3- dimethylureido)benzyl)-3-ethyl- 5,5-dimethyl- 5,6,7,8-tetrahydroimidazo[1,5-a] pyrazine-1-carboxamide |
| V17A | 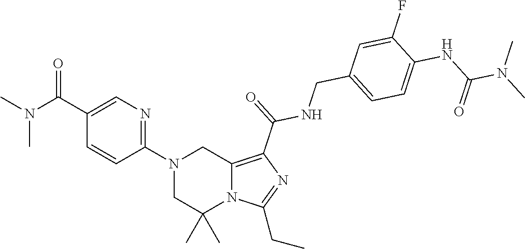 | 7-(5-(dimethylcarbamoyl)pyridin- 2-yl)-N-(4-(3,3-dimethylureido)-3- fluorobenzyl)-3-ethyl-5,5-dimethyl- 5,6,7,8-tetrahydroimidazo[1,5-a] pyrazine-1-carboxamide |
| V18A | 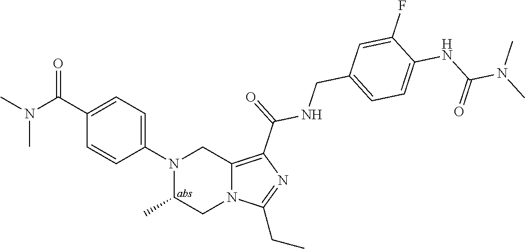 | (S)-7-(4- (dimethylcarbamoyl)phenyl)-N-(4- (3,3-dimethylureido)-3- fluorobenzyl)-3-ethyl-6-methyl- 5,6,7,8-tetrahydroimidazo[1,5-a] pyrazine-1-carboxamide |
| V19A | 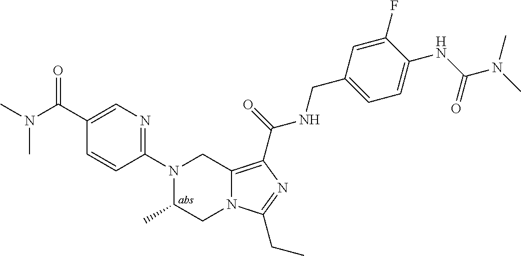 | (S)-7-(5- (dimethylcarbamoyl)pyridin-2-yl)- N-(4-(3,3-dimethylureido)-3- fluorobenzyl)-3-ethyl-6-methyl- 5,6,7,8-tetrahydroimidazo[1,5-a] pyrazine-1-carboxamide |
| V20A | 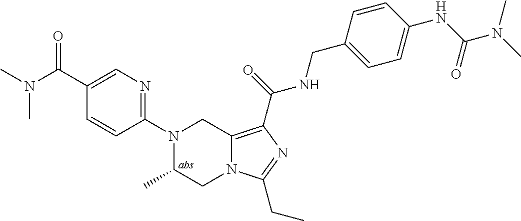 | (S)-7-(5- (dimethylcarbamoyl)pyridin-2-yl)- N-(4-(3,3-dimethylureido)benzyl)- 3-ethyl-6-methyl- 5,6,7,8-tetrahydroimidazo[1,5-a] pyrazine-1-carboxamide |
| V21A | 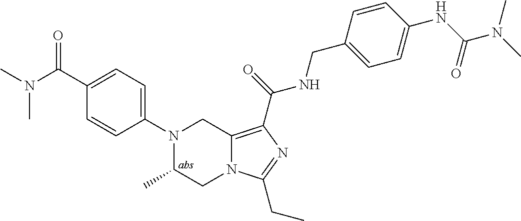 | (S)-7-(4- (dimethylcarbamoyl)phenyl)-N-(4- (3,3-dimethylureido)benzyl)-3- ethyl-6-methyl- 5,6,7,8-tetrahydroimidazo[1,5-a] pyrazine-1-carboxamide |
| V22A | 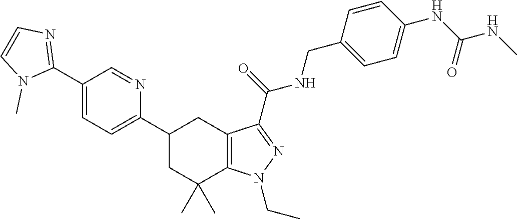 [SMILES: CCn1nc(C(=O)NCc2ccc(NC(=O)NC)cc2)c2c1C(C)(C)CC(c1ccc(-c3nccn3C)cn1)C2] | 1-ethyl-7,7-dimethyl-5-(5-(1- methyl-1H-imidazol-2-yl)pyridin-2- yl)-N-(4-(3-methylureido)benzyl)- 4,5,6,7-tetrahydro-1H-indazole-3- carboxamide |
| V23A | 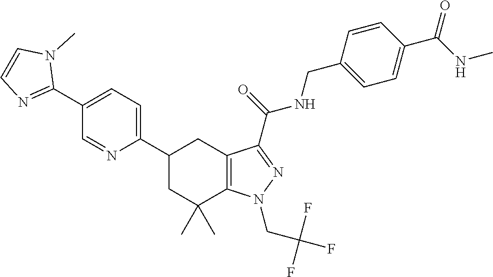 [SMILES: CNC(=O)c1ccc(CNC(=O)c2nn(CC(F)(F)F)c3c2CC(c2ccc(-c4nccn4C)cn2)CC3(C)C)cc1] | 7,7-dimethyl-5-(5-(1-methyl-1H- imidazol-2-yl)pyridin-2-yl)-N-(4- (methylcarbamoyl)benzyl)-1- (2,2,2-trifluoroethyl)-4,5,6,7- tetrahydro-1H-indazole-3- carboxamide |
| V24A | 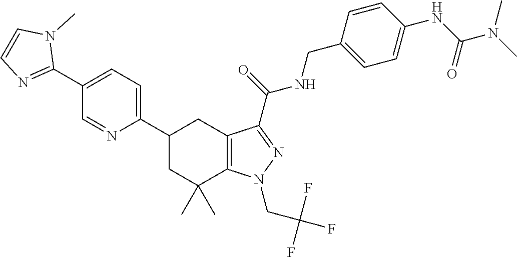 [SMILES: CN(C)C(=O)Nc1ccc(CNC(=O)c2nn(CC(F)(F)F)c3c2CC(c2ccc(-c4nccn4C)cn2)CC3(C)C)cc1] | N-(4-(3,3-dimethylureido)benzyl)- 7,7-dimethyl-5-(5-(1-methyl-1H- imidazol-2-yl)pyridin-2-yl)-1- (2,2,2-trifluoroethyl)-4,5,6,7- tetrahydro-1H-indazole-3- carboxamide |
| V25A | 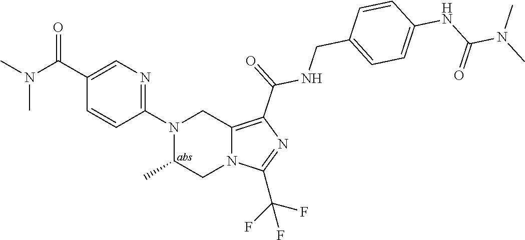 | (S)-7-(5- (dimethylcarbamoyl)pyridin-2-yl)- N-(4-(3,3-dimethylureido)benzyl)- 6-methyl-3-(trifluoromethyl)- 5,6,7,8-tetrahydroimidazo[1,5-a] pyrazine-1-carboxamide |
| V26A | 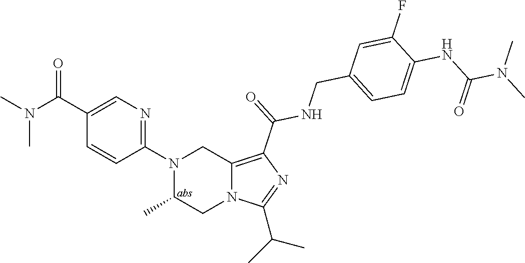 | (S)-7-(5- (dimethylcarbamoyl)pyridin-2-yl)- N-(4-(3,3-dimethylureido)-3- fluorobenzyl)-3-isopropyl-6- methyl- 5,6,7,8-tetrahydroimidazo[1,5-a] pyrazine-1-carboxamide |
| V27A | 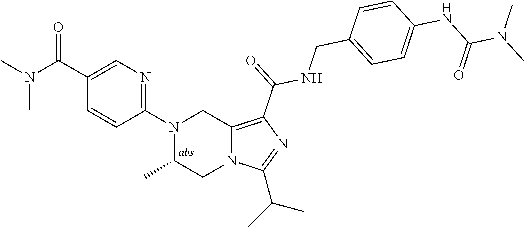 | (S)-7-(5- (dimethylcarbamoyl)pyridin-2-yl)- N-(4-(3,3-dimethylureido)benzyl)- 3-isopropyl-6-methyl- 5,6,7,8-tetrahydroimidazo[1,5-a] pyrazine-1-carboxamide |
| V28A | 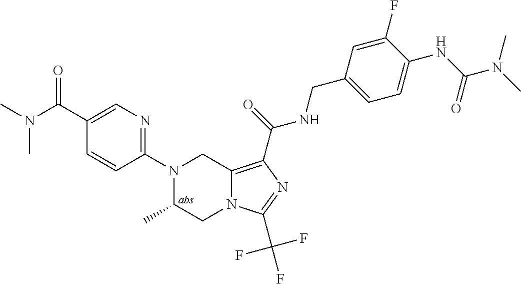 | (S)-7-(5- (dimethylcarbamoyl)pyridin-2-yl)- N-(4-(3,3-dimethylureido)-3- fluorobenzyl)-6-methyl-3- (trifluoromethyl)- 5,6,7,8-tetrahydroimidazo[1,5-a] pyrazine-1-carboxamide |
| V29A | 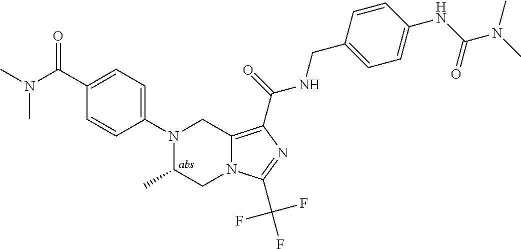 | (S)-7-(4- (dimethylcarbamoyl)phenyl)-N-(4- (3,3-dimethylureido)benzyl)-6- methyl-3-(trifluoromethyl)- 5,6,7,8-tetrahydroimidazo[1,5-a] pyrazine-1-carboxamide |
| V30A | 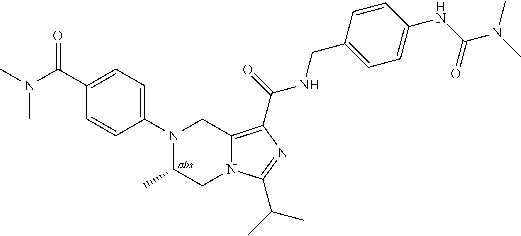 | (S)-7-(4- (dimethylcarbamoyl)phenyl)-N-(4- (3,3-dimethylureido)benzyl)-3- isopropyl-6-methyl- 5,6,7,8-tetrahydroimidazo[1,5-a] pyrazine-1-carboxamide |
| V31A | 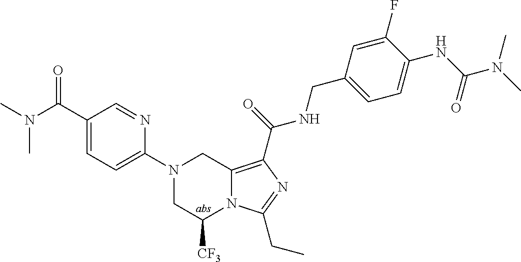 | (R)-7-(5- (dimethylcarbamoyl)pyridin-2-yl)- N-(4-(3,3-dimethylureido)-3- fluorobenzyl)-3-ethyl-5- (trifluoromethyl)- 5,6,7,8-tetrahydroimidazo[1,5-a] pyrazine-1-carboxamide |
| V32A | 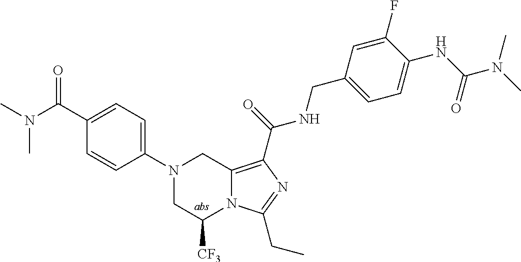 | (R)-7-(4- (dimethylcarbamoyl)phenyl)-N-(4- (3,3-dimethylureido)-3- fluorobenzyl)-3-ethyl-5- (trifluoromethyl)- 5,6,7,8-tetrahydroimidazo[1,5-a] pyrazine-1-carboxamide |
| V33A | 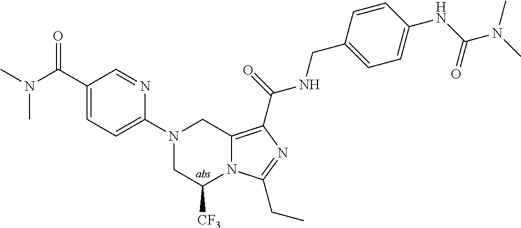 | (R)-7-(5- (dimethylcarbamoyl)pyridin-2-yl)- N-(4-(3,3-dimethylureido)benzyl)- 3-ethyl-5-(trifluoromethyl)- 5,6,7,8-tetrahydroimidazo[1,5-a] pyrazine-1-carboxamide |
| V34A | 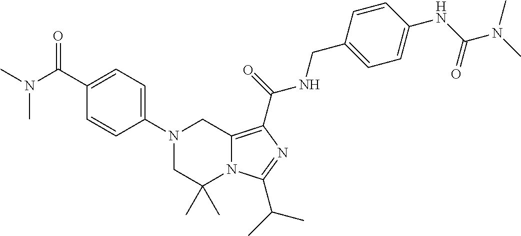 | 7-(4-(dimethylcarbamoyl)phenyl)- N-(4-(3,3-dimethylureido)benzyl)- 3-isopropyl-5,5-dimethyl- 5,6,7,8-tetrahydroimidazo[1,5-a] pyrazine-1-carboxamide |
| V35A | 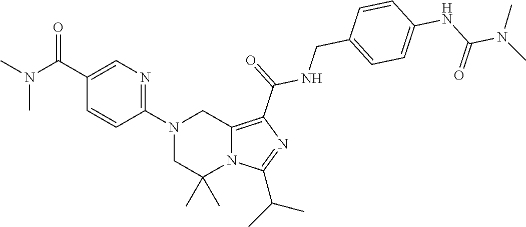 | 7-(5-(dimethylcarbamoyl)pyridin- 2-yl)-N-(4-(3,3- dimethylureido)benzyl)-3- isopropyl-5,5-dimethyl- 5,6,7,8-tetrahydroimidazo[1,5-a] pyrazine-1-carboxamide |
| V36A | 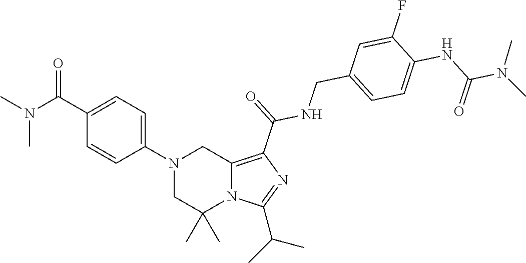 | 7-(4-(dimethylcarbamoyl)phenyl)- N-(4-(3,3-dimethylureido)-3- fluorobenzyl)-3-isopropyl-5,5- dimethyl-5,6,7,8- tetrahydroimidazo[1,5-a] pyrazine-1-carboxamide |
| V37A | 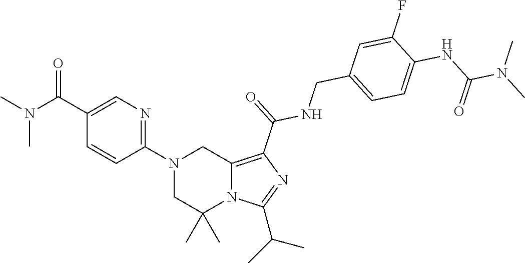 | 7-(5-(dimethylcarbamoyl)pyridin- 2-yl)-N-(4-(3,3-dimethylureido)-3- fluorobenzyl)-3-isopropyl-5,5- dimethyl- 5,6,7,8-tetrahydroimidazo[1,5-a] pyrazine-1-carboxamide |
| V38A | 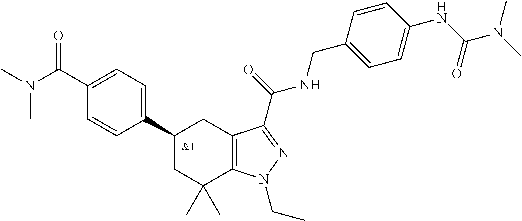 | rac-(R)-5-(4- (dimethylcarbamoyl)phenyl)-N-(4- (3,3-dimethylureido)benzyl)-1- ethyl-7,7-dimethyl-4,5,6,7- tetrahydro-1H-indazole-3- carboxamide |
| V39A | 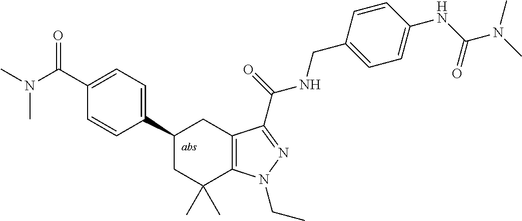 | (R)-5-(4- (dimethylcarbamoyl)phenyl)-N-(4- (3,3-dimethylureido)benzyl)-1- ethyl-7,7-dimethyl-4,5,6,7- tetrahydro-1H-indazole-3- carboxamide |
| V40A | 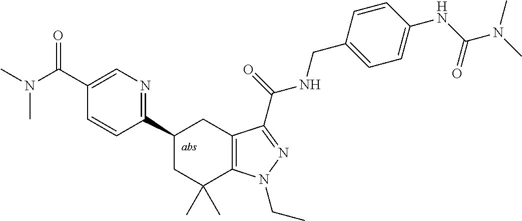 | (R)-5-(5- (dimethylcarbamoyl)pyridin-2-yl)- N-(4-(3,3-dimethylureido)benzyl)- 1-ethyl-7,7-dimethyl-4,5,6,7- tetrahydro-1H-indazole-3- carboxamide |
| V41A | 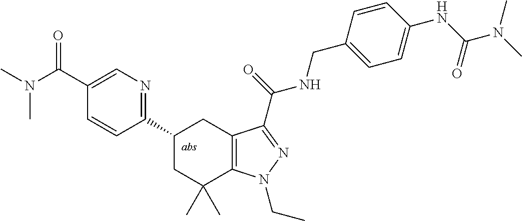 | (S)-5-(5- (dimethylcarbamoyl)pyridin-2-yl)- N-(4-(3,3-dimethylureido)benzyl)- 1-ethyl-7,7-dimethyl-4,5,6,7- tetrahydro-1H-indazole-3- carboxamide |
| V42A | 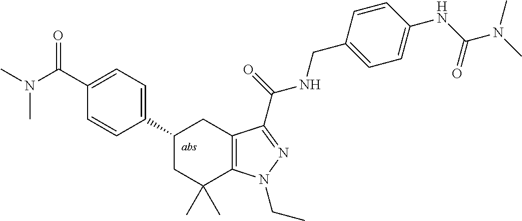 | (S)-5-(4- (dimethylcarbamoyl)phenyl)-N-(4- (3,3-dimethylureido)benzyl)-1- ethyl-7,7-dimethyl-4,5,6,7- tetrahydro-1H-indazole-3- carboxamide |
| V43A | 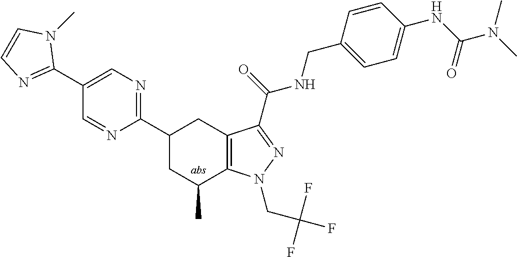 [SMILES: C[C@H]1CC(c2ncc(-c3nccn3C)cn2)Cc2c(C(=O)NCc3ccc(NC(=O)N(C)C)cc3)nn(CC(F)(F)F)c21] | (7S)-N-(4-(3,3- dimethylureido)benzyl)-7-methyl- 5-(5-(1-methyl-1H-imidazol-2- yl)pyrimidin-2-yl)-1-(2,2,2- trifluoroethyl)-4,5,6,7- tetrahydro-1H-indazole-3- carboxamide |
| V44A | 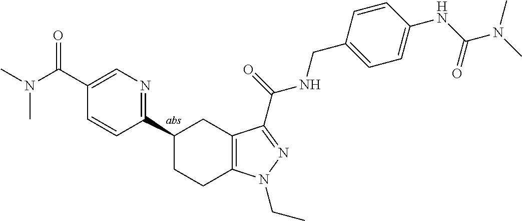 | (R)-5-(5- (dimethylcarbamoyl)pyridin-2-yl)- N-(4-(3,3-dimethylureido)benzyl)- 1-ethyl-4,5,6,7- tetrahydro-1H-indazole-3- carboxamide |
| V45A | 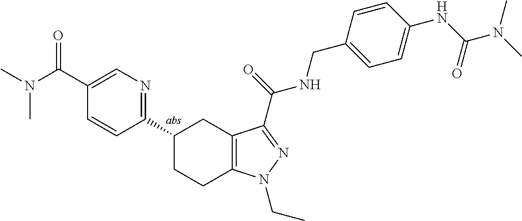 | (S)-5-(5- (dimethylcarbamoyl)pyridin-2-yl)- N-(4-(3,3-dimethylureido)benzyl)- 1-ethyl-4,5,6,7- tetrahydro-1H-indazole-3- carboxamide |
| V46A | 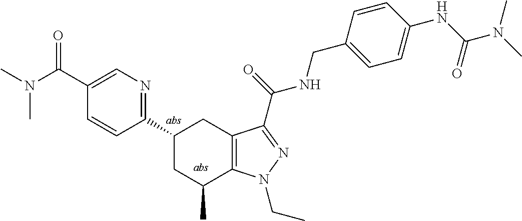 | (5S,7S)-5-(5- (dimethylcarbamoyl)pyridin-2-yl)- N-(4-(3,3-dimethylureido)benzyl)- 1-ethyl-7-methyl-4,5,6,7- tetrahydro-1H-indazole-3- carboxamide |
| V47A | 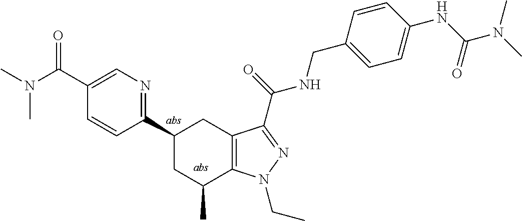 | (5R,7S)-5-(5- (dimethylcarbamoyl)pyridin-2-yl)- N-(4-(3,3-dimethylureido)benzyl)- 1-ethyl-7-methyl-4,5,6,7- tetrahydro-1H-indazole-3- carboxamide |
| V48A | 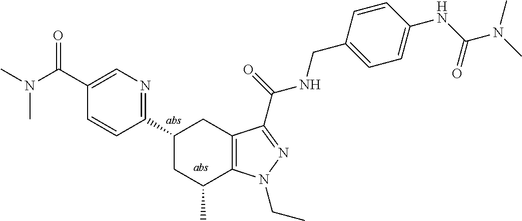 | (5S,7R)-5-(5- (dimethylcarbamoyl)pyridin-2-yl)- N-(4-(3,3-dimethylureido)benzyl)- 1-ethyl-7-methyl-4,5,6,7- tetrahydro-1H-indazole-3- carboxamide |
| V49A | 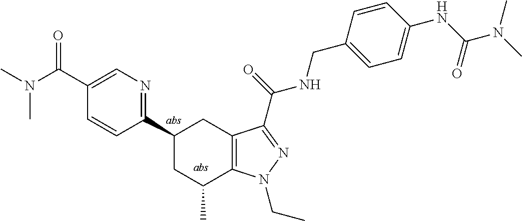 | (5R,7R)-5-(5- (dimethylcarbamoyl)pyridin-2-yl)- N-(4-(3,3-dimethylureido)benzyl)- 1-ethyl-7-ethyl-4,5,6,7- tetrahydro-1H-indazole-3- carboxamide |
| V50A | 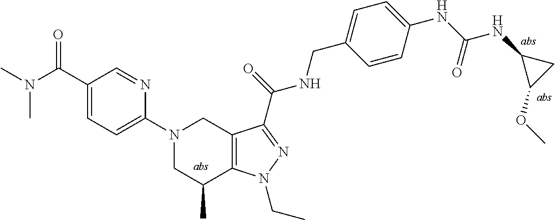 [SMILES: CCn1nc(C(=O)NCc2ccc(NC(=O)N[C@H]3C[C@@H]3OC)cc2)c2c1[C@@H](C)CN(c1ccc(C(=O)N(C)C)cn1)C2] | (S)-5-(5- (dimethylcarbamoyl)pyridin-2-yl)- 1-ethyl-N-(4-(3-((1S,2S)-2- methoxycyclopropyl)ureido) benzyl)-7-methyl-4,5,6,7- tetrahydro-1H-pyrazolo[4,3-c] pyridine-3-carboxamide |
| V51A | 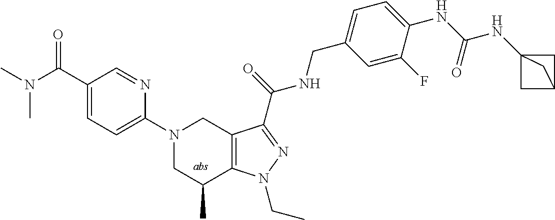 [SMILES: CCn1nc(C(=O)NCc2ccc(NC(=O)NC34CC(C3)C4)c(F)c2)c2c1[C@@H](C)CN(c1ccc(C(=O)N(C)C)cn1)C2] | (S)-N-(4-(3-(bicyclo[1.1.1]pentan- 1-yl)ureido)-3-fluorobenzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V52A | 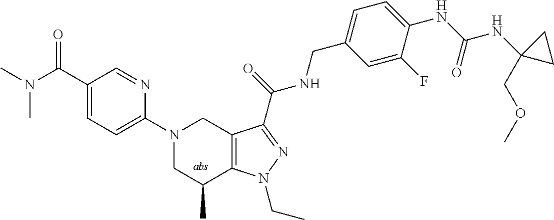 [SMILES: CCn1nc(C(=O)NCc2ccc(NC(=O)NC3(COC)CC3)c(F)c2)c2c1[C@@H](C)CN(c1ccc(C(=O)N(C)C)cn1)C2] | (S)-5-(5- (dimethylcarbamoyl)pyridin-2-yl)- 1-ethyl-N-(3-fluoro-4-(3-(1- (methoxymethyl)cyclopropyl) ureido)benzyl)-7-methyl-4,5,6,7- tetrahydro-1H-pyrazolo[4,3-c] pyridine-3-carboxamide |
| V53A | 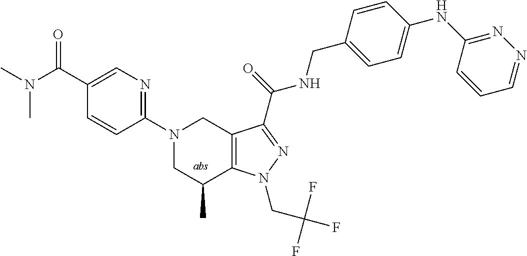 [SMILES: C[C@H]1CN(c2ccc(C(=O)N(C)C)cn2)Cc2c(C(=O)NCc3ccc(Nc4cccnn4)cc3)nn(CC(F)(F)F)c21] | (S)-5-(5- (dimethylcarbamoyl)pyridin-2-yl)- 7-methyl-N-(4-(pyridazin-3- ylamino)benzyl)-1-(2,2,2- trifluoroethyl)-4,5,6,7- tetrahydro-1H-pyrazolo[4,3-c] pyridine-3-carboxamide |
| V54A | 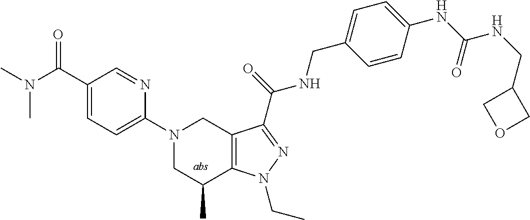 [SMILES: CCn1nc(C(=O)NCc2ccc(NC(=O)NCC3COC3)cc2)c2c1[C@@H](C)CN(c1ccc(C(=O)N(C)C)cn1)C2] | (S)-5-(5- (dimethylcarbamoyl)pyridin-2-yl)- 1-ethyl-7-methyl-N-(4-(3-(oxetan- 3-ylmethyl)ureido)benzyl)-4,5,6,7- tetrahydro-1H-pyrazolo[4,3- c]pyridine-3-carboxamide |
| V55A | 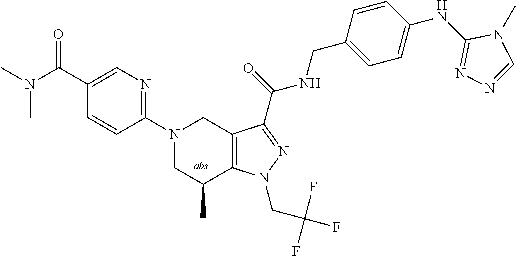 | (S)-5-(5- (dimethylcarbamoyl)pyridin-2-yl)- 7-methyl-N-(4-((4-methyl-4H- 1,2,4-triazol-3-yl)amino)benzyl)-1- (2,2,2-trifluoroethyl)-4,5,6,7- tetrahydro-1H-pyrazolo[4,3- c]pyridine-3-carboxamide |
| V56A | 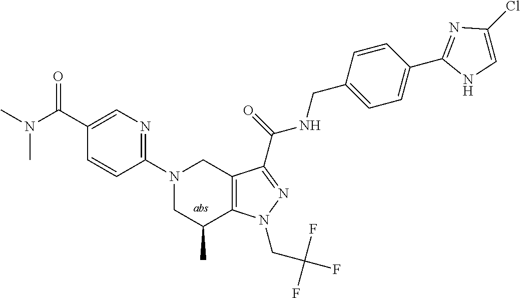 [SMILES: C[C@H]1CN(c2ccc(C(=O)N(C)C)cn2)Cc2c(C(=O)NCc3ccc(-c4nc(Cl)c[nH]4)cc3)nn(CC(F)(F)F)c21] | (S)-N-(4-(4-chloro-1H-imidazol-2- yl)benzyl)-5-(5- (dimethylcarbamoyl)pyridin-2-yl)- 7-methyl-1-(2,2,2-trifluoroethyl)- 4,5,6,7- tetrahydro-1H-pyrazolo[4,3- c]pyridine-3-carboxamide |
| V57A | 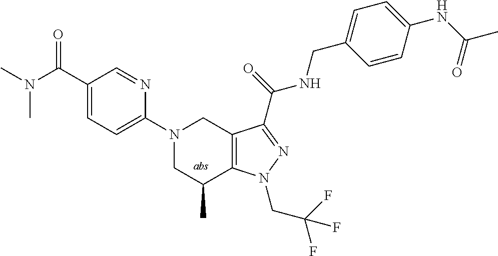 [SMILES: CC(=O)Nc1ccc(CNC(=O)c2nn(CC(F)(F)F)c3c2CN(c2ccc(C(=O)N(C)C)cn2)C[C@@H]3C)cc1] | (S)-N-(4-acetamidobenzyl)-5-(5- (dimethylcarbamoyl)pyridin-2-yl)- 7-methyl-1-(2,2,2-trifluoroethyl)- 4,5,6,7- tetrahydro-1H-pyrazolo[4,3- c]pyridine-3-carboxamide |
| V58A | 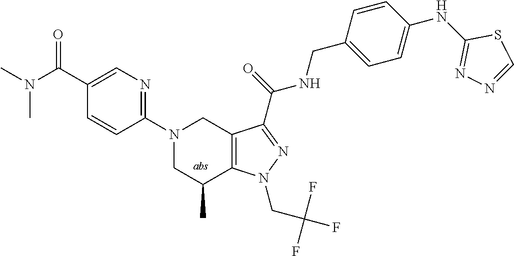 [SMILES: C[C@H]1CN(c2ccc(C(=O)N(C)C)cn2)Cc2c(C(=O)NCc3ccc(Nc4nncs4)cc3)nn(CC(F)(F)F)c21] | (S)-N-(4-((1,3,4-thiadiazol-2- yl)amino)benzyl)-5-(5- (dimethylcarbamoyl)pyridin-2-yl)- 7-methyl-1-(2,2,2-trifluoroethyl)- 4,5,6,7- tetrahydro-1H-pyrazolo[4,3- c]pyridine-3-carboxamide |
| V59A | 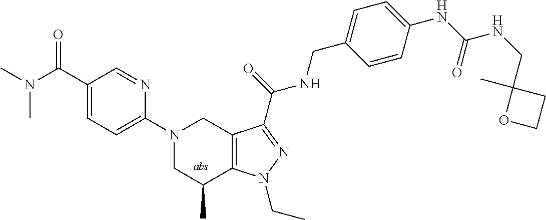 [SMILES: CCn1nc(C(=O)NCc2ccc(NC(=O)NCC3(C)CCO3)cc2)c2c1[C@@H](C)CN(c1ccc(C(=O)N(C)C)cn1)C2] | (7S)-5-(5- (dimethylcarbamoyl)pyridin-2-yl)- 1-ethyl-7-methyl-N-(4-(3-((2- methyloxetan-2- yl)methyl)ureido)benzyl)-4,5,6,7- tetrahydro-1H-pyrazolo[4,3-c] pyridine-3-carboxamide |
| V60A | 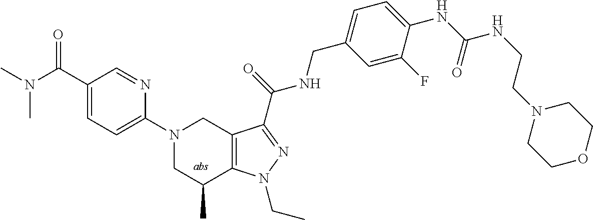 [SMILES: CCn1nc(C(=O)NCc2ccc(NC(=O)NCCN3CCOCC3)c(F)c2)c2c1[C@@H](C)CN(c1ccc(C(=O)N(C)C)cn1)C2] | (S)-5-(5- (dimethylcarbamoyl)pyridin-2-yl)- 1-ethyl-N-(3-fluoro-4-(3-(2- morpholinoethyl)ureido)benzyl)-7- methyl-4,5,6,7- tetrahydro-1H-pyrazolo[4,3-c] pyridine-3-carboxamide |
| V61A | 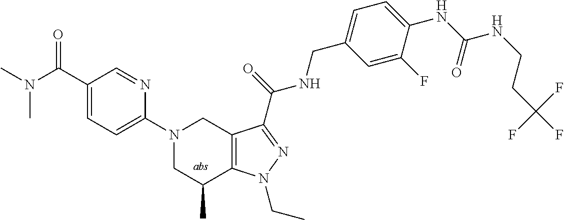 [SMILES: CCn1nc(C(=O)NCc2ccc(NC(=O)NCCC(F)(F)F)c(F)c2)c2c1[C@@H](C)CN(c1ccc(C(=O)N(C)C)cn1)C2] | (S)-5-(5- (dimethylcarbamoyl)pyridin-2-yl)- 1-ethyl-N-(3-fluoro-4-(3-(3,3,3- trifluoropropyl)ureido)benzyl)-7- methyl-4,5,6,7- tetrahydro-1H-pyrazolo[4,3-c] pyridine-3-carboxamide |
| V62A | 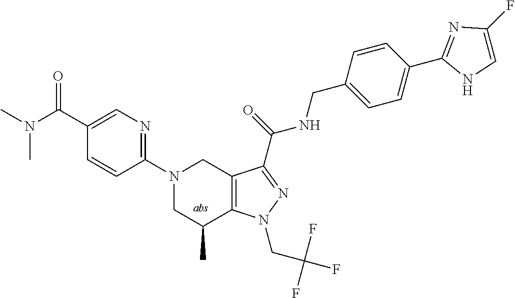 [SMILES: C[C@H]1CN(c2ccc(C(=O)N(C)C)cn2)Cc2c(C(=O)NCc3ccc(-c4nc(F)c[nH]4)cc3)nn(CC(F)(F)F)c21] | (S)-5-(5- (dimethylcarbamoyl)pyridin-2-yl)- N-(4-(4-fluoro-1H-imidazol-2- yl)benzyl)-7-methyl-1-(2,2,2- trifluoroethyl)-4,5,6,7- tetrahydro-1H-pyrazolo[4,3-c] pyridine-3-carboxamide |
| V63A | 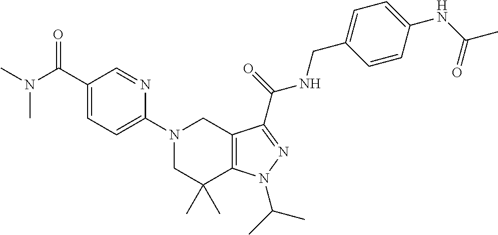 [SMILES: CC(=O)Nc1ccc(CNC(=O)c2nn(C(C)C)c3c2CN(c2ccc(C(=O)N(C)C)cn2)CC3(C)C)cc1] | N-(4-acetamidobenzyl)-5-(5- (dimethylcarbamoyl)pyridin-2-yl)- 1-isopropyl-7,7-dimethyl-4,5,6,7- tetrahydro-1H-pyrazolo[4,3-c] pyridine-3-carboxamide |
| V64A | 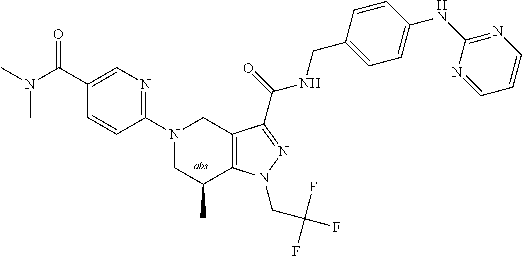 [SMILES: C[C@H]1CN(c2ccc(C(=O)N(C)C)cn2)Cc2c(C(=O)NCc3ccc(Nc4ncccn4)cc3)nn(CC(F)(F)F)c21] | (S)-5-(5- (dimethylcarbamoyl)pyridin-2-yl)- 7-methyl-N-(4-(pyrimidin-2- ylamino)benzyl)-1-(2,2,2- trifluoroethyl)-4,5,6,7- tetrahydro-1H-pyrazolo[4,3-c] pyridine-3-carboxamide |
| V65A | 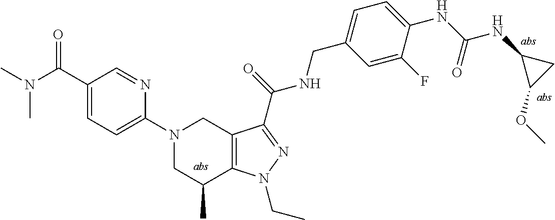 [SMILES: CCn1nc(C(=O)NCc2ccc(NC(=O)N[C@H]3C[C@@H]3OC)c(F)c2)c2c1[C@@H](C)CN(c1ccc(C(=O)N(C)C)cn1)C2] | (S)-5-(5- (dimethylcarbamoyl)pyridin-2-yl)- 1-ethyl-N-(3-fluoro-4-(3-((1S,2S)- 2-methoxycyclopropyl)ureido) benzyl)-7-methyl-4,5,6,7- tetrahydro-1H-pyrazolo[4,3-c] pyridine-3-carboxamide |
| V66A | 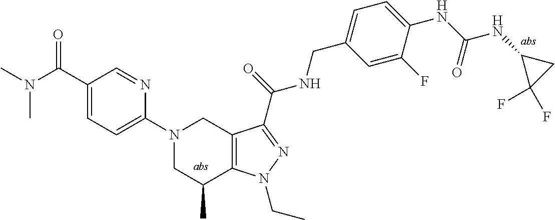 [SMILES: CCn1nc(C(=O)NCc2ccc(NC(=O)N[C@@H]3CC3(F)F)c(F)c2)c2c1[C@@H](C)CN(c1ccc(C(=O)N(C)C)cn1)C2] | (S)-N-(4-(3-((R)-2,2- difluorocyclopropyl)ureido)-3- fluorobenzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V67A | 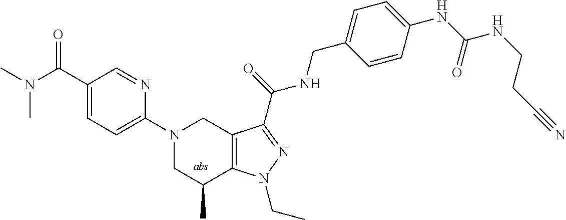 [SMILES: CCn1nc(C(=O)NCc2ccc(NC(=O)NCCC#N)cc2)c2c1[C@@H](C)CN(c1ccc(C(=O)N(C)C)cn1)C2] | (S)-N-(4-(3-(2- cyanoethyl)ureido)benzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V68A | 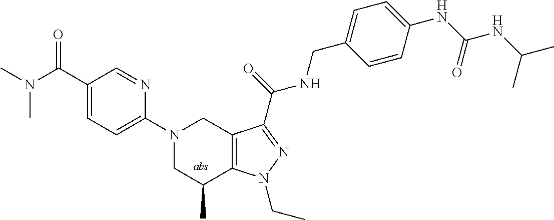 [SMILES: CCn1nc(C(=O)NCc2ccc(NC(=O)NC(C)C)cc2)c2c1[C@@H](C)CN(c1ccc(C(=O)N(C)C)cn1)C2] | (S)-5-(5- (dimethylcarbamoyl)pyridin-2-yl)- 1-ethyl-N-(3-fluoro-4-(3- isopropylureido)benzyl)-7-methyl- 4,5,6,7- tetrahydro-1H-pyrazolo[4,3-c] pyridine-3-carboxamide |
| V69A | 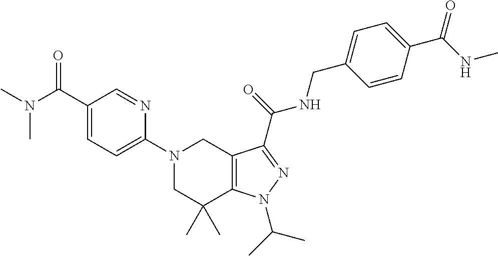 | 5-(5-(dimethylcarbamoyl)pyridin- 2-yl)-1-isopropyl-7,7-dimethyl-N- (4-(methylcarbamoyl)benzyl)- 4,5,6,7- tetrahydro-1H-pyrazolo[4,3-c] pyridine-3-carboxamide |
| V70A | 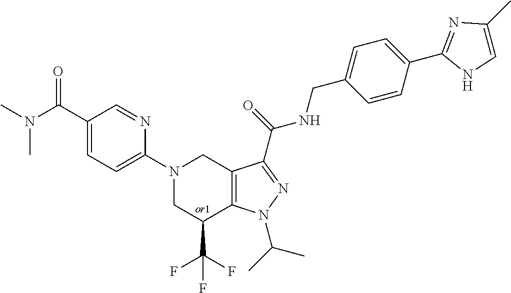 | rel-(R)-5-(5- (dimethylcarbamoyl)pyridin-2-yl)- 1-isopropyl-N-(4-(4-methyl-1H- imidazol-2-yl)benzyl)-7- (trifluoromethyl)-4,5,6,7- tetrahydro-1H-pyrazolo[4,3-c] pyridine-3-carboxamide |
| V71A | 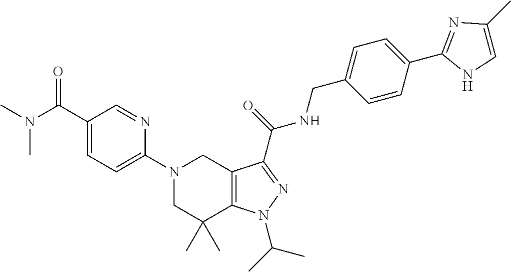 | 5-(5-(dimethylcarbamoyl)pyridin- 2-yl)-1-isopropyl-7,7-dimethyl-N- (4-(4-methyl-1H-imidazol-2- yl)benzyl)-4,5,6,7- tetrahydro-1H-pyrazolo[4,3-c] pyridine-3-carboxamide |
| V72A | 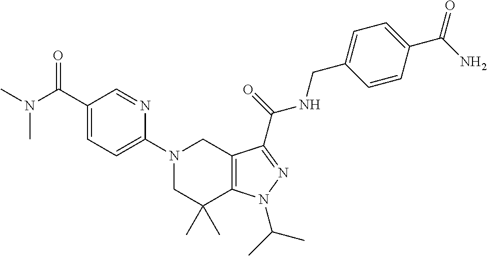 [SMILES: CC(C)n1nc(C(=O)NCc2ccc(C(N)=O)cc2)c2c1C(C)(C)CN(c1ccc(C(=O)N(C)C)cn1)C2] | N-(4-carbamoylbenzyl)-5-(5- (dimethylcarbamoyl)pyridin-2-yl)- 1-isopropyl-7,7-dimethyl- 4,5,6,7- tetrahydro-1H-pyrazolo[4,3-c] pyridine-3-carboxamide |
| V73A | 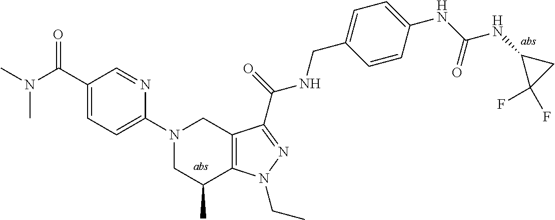 [SMILES: CCn1nc(C(=O)NCc2ccc(NC(=O)N[C@@H]3CC3(F)F)cc2)c2c1[C@@H](C)CN(c1ccc(C(=O)N(C)C)cn1)C2] | (S)-N-(4-(3-((R)-2,2- difluorocyclopropyl)ureido)benzyl)- 5-(5-(dimethylcarbamoyl)pyridin- 2-yl)-1-ethyl-7-methyl-4,5,6,7- tetrahydro-1H-pyrazolo[4,3-c] pyridine-3-carboxamide |
| V74A | 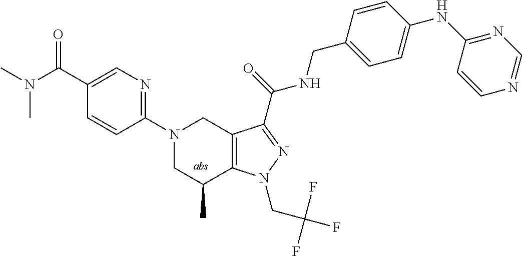 [SMILES: C[C@H]1CN(c2ccc(C(=O)N(C)C)cn2)Cc2c(C(=O)NCc3ccc(Nc4ccncn4)cc3)nn(CC(F)(F)F)c21] | (S)-5-(5- (dimethylcarbamoyl)pyridin-2-yl)- 7-methyl-N-(4-(pyrimidin-4- ylamino)benzyl)-1-(2,2,2- trifluoroethyl)-4,5,6,7- tetrahydro-1H-pyrazolo[4,3-c] pyridine-3-carboxamide |
| V75A | 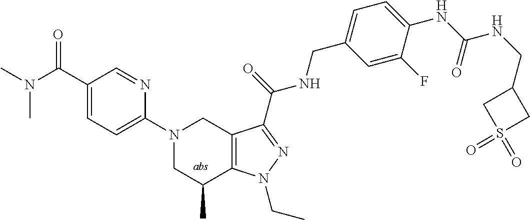 [SMILES: CCn1nc(C(=O)NCc2ccc(NC(=O)NCC3CS(=O)(=O)C3)c(F)c2)c2c1[C@@H](C)CN(c1ccc(C(=O)N(C)C)cn1)C2] | (S)-5-(5- (dimethylcarbamoyl)pyridin-2-yl)- N-(4-(3-((1,1-dioxidothietan-3- yl)methyl)ureido)-3-fluorobenzyl)- 1-ethyl-7-methyl-4,5,6,7- tetrahydro-1H-pyrazolo[4,3-c] pyridine-3-carboxamide |
| V76A | 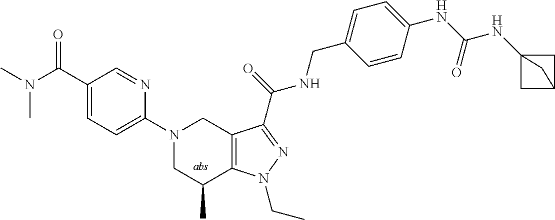 [SMILES: CCn1nc(C(=O)NCc2ccc(NC(=O)NC34CC(C3)C4)cc2)c2c1[C@@H](C)CN(c1ccc(C(=O)N(C)C)cn1)C2] | (S)-N-(3-(bicyclo[1.1.1]pentan- 1-yl)ureido)benzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V77A | 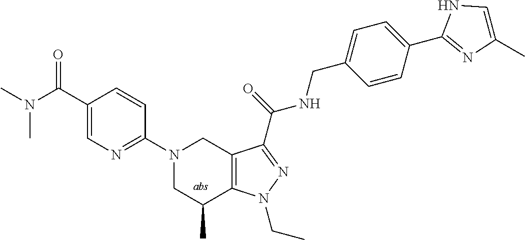 | (S)-5-(5- (dimethylcarbamoyl)pyridin-2-yl)- 1-ethyl-7-methyl-N-(4-(4-methyl- 1H-imidazol-2-yl)benzyl)-4,5,6,7- tetrahydro-1H-pyrazolo[4,3-c] pyridine-3-carboxamide |
| V78A | 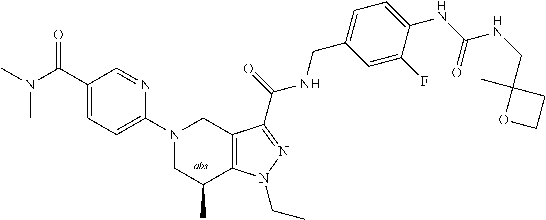 [SMILES: CCn1nc(C(=O)NCc2ccc(NC(=O)NCC3(C)CCO3)c(F)c2)c2c1[C@@H](C)CN(c1ccc(C(=O)N(C)C)cn1)C2] | (7S)-5-(5- (dimethylcarbamoyl)pyridin-2-yl)- 1-ethyl-N-(3-fluoro-4-(3-((2- methyloxetan-2- yl)methyl)ureido)benzyl)-7-methyl- 4,5,6,7- tetrahydro-1H-pyrazolo[4,3-c] pyridine-3-carboxamide |
| V79A | 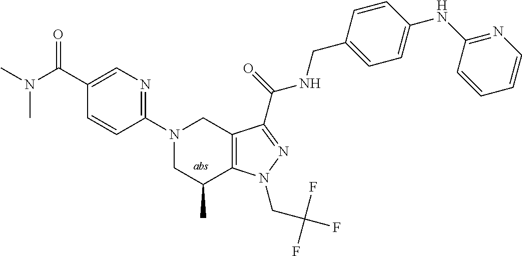 [SMILES: C[C@H]1CN(c2ccc(C(=O)N(C)C)cn2)Cc2c(C(=O)NCc3ccc(Nc4ccccn4)cc3)nn(CC(F)(F)F)c21] | (S)-5-(5- (dimethylcarbamoyl)pyridin-2-yl)- 7-methyl-N-(4-(pyridin-2- ylamino)benzyl)-1-(2,2,2- trifluoroethyl)-4,5,6,7- tetrahydro-1H-pyrazolo[4,3-c] pyridine-3-carboxamide |
| V80A | 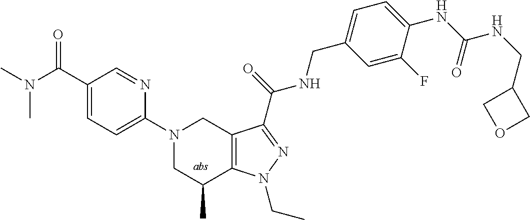 [SMILES: CCn1nc(C(=O)NCc2ccc(NC(=O)NCC3COC3)c(F)c2)c2c1[C@@H](C)CN(c1ccc(C(=O)N(C)C)cn1)C2] | (S)-5-(5- (dimethylcarbamoyl)pyridin-2-yl)- 1-ethyl-N-(3-fluoro-4-(3-(oxetan-3- ylmethyl)ureido)benzyl)-7-methyl- 4,5,6,7- tetrahydro-1H-pyrazolo[4,3-c] pyridine-3-carboxamide |
| V81A | 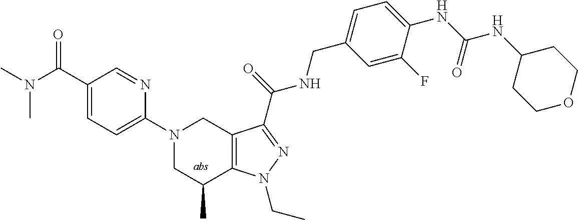 [SMILES: CCn1nc(C(=O)NCc2ccc(NC(=O)NC3CCOCC3)c(F)c2)c2c1[C@@H](C)CN(c1ccc(C(=O)N(C)C)cn1)C2] | (S)-5-(5- (dimethylcarbamoyl)pyridin-2-yl)- 1-ethyl-N-(3-fluoro-4-(3- (tetrahydro-2H-pyran-4- yl)ureido)benzyl)-7-methyl- 4,5,6,7- tetrahydro-1H-pyrazolo[4,3-c] pyridine-3-carboxamide |
| V82A | 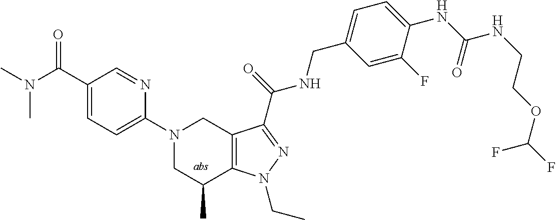 [SMILES: CCn1nc(C(=O)NCc2ccc(NC(=O)NCCOC(F)F)c(F)c2)c2c1[C@@H](C)CN(c1ccc(C(=O)N(C)C)cn1)C2] | (S)-N-(4-(3-(2- (difluoromethoxy)ethyl)ureido)-3- fluorobenzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V83A | 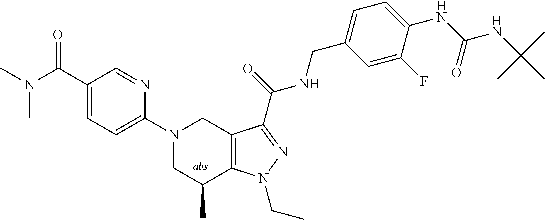 [SMILES: CCn1nc(C(=O)NCc2ccc(NC(=O)NC(C)(C)C)c(F)c2)c2c1[C@@H](C)CN(c1ccc(C(=O)N(C)C)cn1)C2] | (S)-N-(4-(3-(tert-butyl)ureido)-3- fluorobenzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V84A | 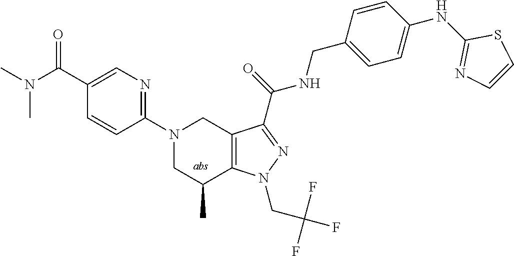 [SMILES: C[C@H]1CN(c2ccc(C(=O)N(C)C)cn2)Cc2c(C(=O)NCc3ccc(Nc4nccs4)cc3)nn(CC(F)(F)F)c21] | (S)-5-(5- (dimethylcarbamoyl)pyridin-2-yl)- 7-methyl-N-(4-(thiazol-2- ylamino)benzyl)-1-(2,2,2- trifluoroethyl)-4,5,6,7- tetrahydro-1H-pyrazolo[4,3-c] pyridine-3-carboxamide |
| V85A | 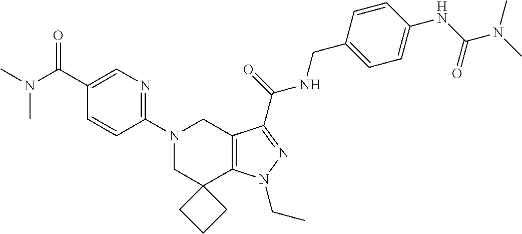 | 5′-(5-(dimethylcarbamoyl)pyridin- 2-yl)-N-(4-(3,3- dimethylureido)benzyl)-1′-ethyl- 1′,4′,5′,6′- tetrahydrospiro[cyclobutane-1,7′- pyrazolo[4,3-c]pyridine]-3′- carboxamide |
| V86A | 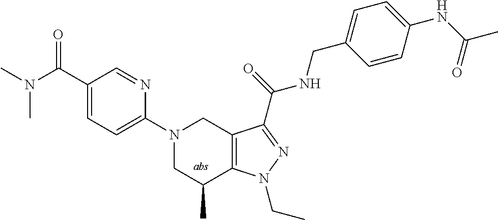 [SMILES: CCn1nc(C(=O)NCc2ccc(NC(C)=O)cc2)c2c1[C@@H](C)CN(c1ccc(C(=O)N(C)C)cn1)C2] | (S)-N-(4-acetamidobenzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V87A | 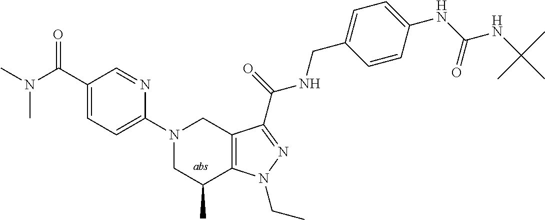 [SMILES: CCn1nc(C(=O)NCc2ccc(NC(=O)NC(C)(C)C)cc2)c2c1[C@@H](C)CN(c1ccc(C(=O)N(C)C)cn1)C2] | (S)-N-(4-(3-(tert- butyl)ureido)benzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V88A | 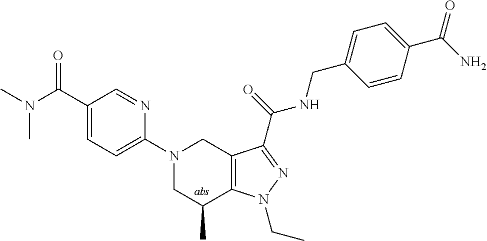 [SMILES: CCn1nc(C(=O)NCc2ccc(C(N)=O)cc2)c2c1[C@@H](C)CN(c1ccc(C(=O)N(C)C)cn1)C2] | (S)-N-(4-carbamoylbenzyl)-5-(5- (dimethylcarbamoyl)pyridin-2-yl)- 1-ethyl-7-methyl-4,5,6,7- tetrahydro-1H-pyrazolo[4,3-c] pyridine-3-carboxamide |
| V89A | 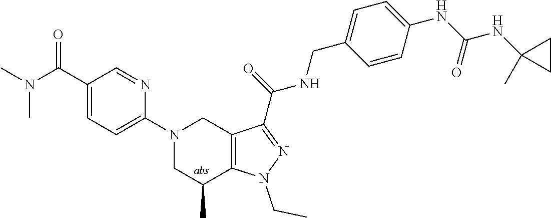 [SMILES: CCn1nc(C(=O)NCc2ccc(NC(=O)NC3(C)CC3)cc2)c2c1[C@@H](C)CN(c1ccc(C(=O)N(C)C)cn1)C2] | (S)-5-(5- (dimethylcarbamoyl)pyridin-2-yl)- 1-ethyl-7-methyl-N-(4-(3-(1- methylcyclopropyl)ureido)benzyl)- 4,5,6,7- tetrahydro-1H-pyrazolo[4,3-c] pyridine-3-carboxamide |
| V90A | 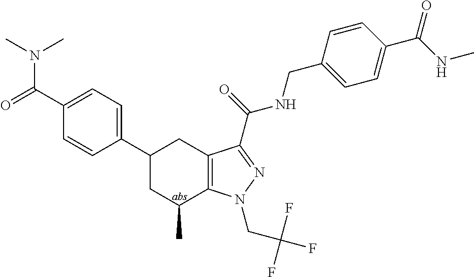 [SMILES: CNC(=O)c1ccc(CNC(=O)c2nn(CC(F)(F)F)c3c2CC(c2ccc(C(=O)N(C)C)cc2)C[C@@H]3C)cc1] | (7S)-5-(4- (dimethylcarbamoyl)phenyl)-7- methyl-N-(4- (methylcarbamoyl)benzyl)-1- (2,2,2-trifluoroethyl)-4,5,6,7- tetrahydro-1H-indazole-3- carboxamide |
| V91A | 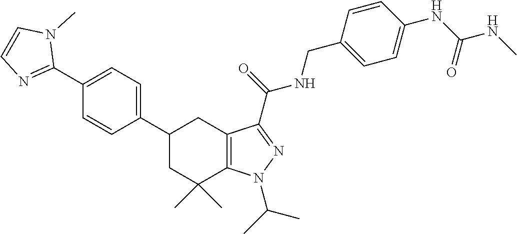 [SMILES: CNC(=O)Nc1ccc(CNC(=O)c2nn(C(C)C)c3c2CC(c2ccc(-c4nccn4C)cc2)CC3(C)C)cc1] | 1-isopropyl-7,7-dimethyl-5-(4-(1- methyl-1H-imidazol-2-yl)phenyl)- N-(4-(3-methylureido)benzyl)- 4,5,6,7- tetrahydro-1H-indazole-3- carboxamide |
| V92A | 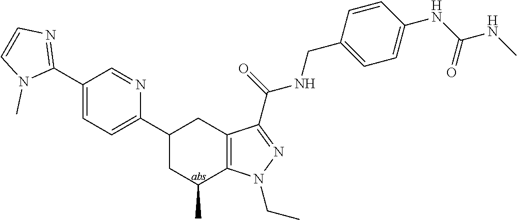 [SMILES: CCn1nc(C(=O)NCc2ccc(NC(=O)NC)cc2)c2c1[C@@H](C)CC(c1ccc(-c3nccn3C)cn1)C2] | (7S)-1-ethyl-7-methyl-5-(5-(1- methyl-1H-imidazol-2-yl)pyridin-2- yl)-N-(4-(3-methylureido)benzyl)- 4,5,6,7- tetrahydro-1H-indazole-3- carboxamide |
| V93A | 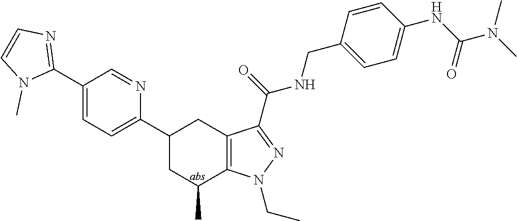 [SMILES: CCn1nc(C(=O)NCc2ccc(NC(=O)N(C)C)cc2)c2c1[C@@H](C)CC(c1ccc(-c3nccn3C)cn1)C2] | (7S)-N-(4-(3,3- dimethylureido)benzyl)-1-ethyl-7- methyl-5-(5-(1-methyl-1H- imidazol-2-yl)pyridin-2-yl)- 4,5,6,7- tetrahydro-1H-indazole-3- carboxamide |
| V94A | 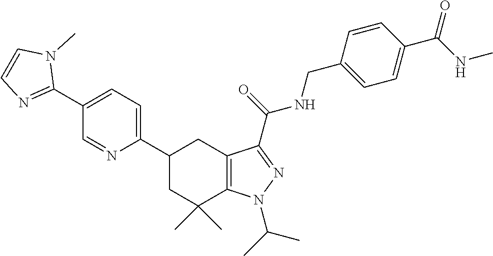 [SMILES: CNC(=O)c1ccc(CNC(=O)c2nn(C(C)C)c3c2CC(c2ccc(-c4nccn4C)cn2)CC3(C)C)cc1] | 1-isopropyl-7,7-dimethyl-5-(5-(1- methyl-1H-imidazol-2-yl)pyridin-2- yl)-N-(4- (methylcarbamoyl)benzyl)- 4,5,6,7- tetrahydro-1H-indazole-3- carboxamide |
| V95A | 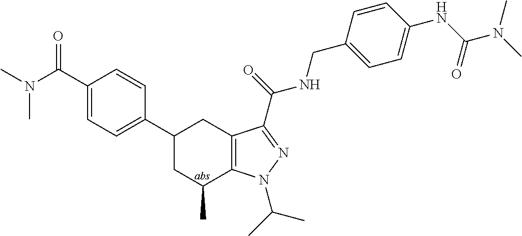 | (7S)-5-(4- (dimethylcarbamoyl)phenyl)-N-(4- (3,3-dimethylureido)benzyl)-1- isopropyl-7-methyl-4,5,6,7- tetrahydro-1H-indazole-3- carboxamide |
| V96A | 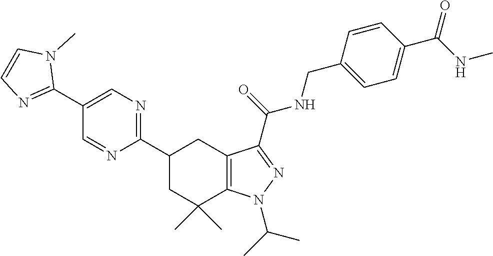 [SMILES: CNC(=O)c1ccc(CNC(=O)c2nn(C(C)C)c3c2CC(c2ncc(-c4nccn4C)cn2)CC3(C)C)cc1] | 1-isopropyl-7,7-dimethyl-5-(5-(1- methyl-1H-imidazol-2-yl) pyrimidin-2-yl)-N-(4- (methylcarbamoyl)benzyl)- 4,5,6,7- tetrahydro-1H-indazole-3- carboxamide |
| V97A | 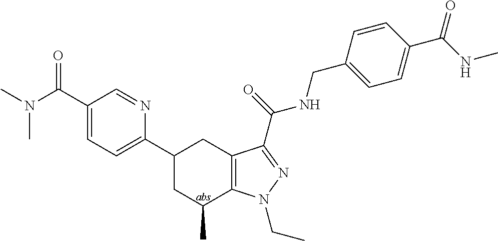 | (7S)-5-(5- (dimethylcarbamoyl)pyridin-2-yl)- 1-ethyl-7-methyl-N-(4- (methylcarbamoyl)benzyl)-4,5,6,7- tetrahydro-1H-indazole-3- carboxamide |
| V98A | 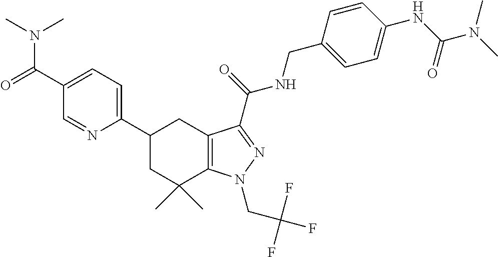 [SMILES: CN(C)C(=O)Nc1ccc(CNC(=O)c2nn(CC(F)(F)F)c3c2CC(c2ccc(C(=O)N(C)C)cn2)CC3(C)C)cc1] | 5-(5-(dimethylcarbamoyl)pyridin- 2-yl)-N-(4-(3,3- dimethylureido)benzyl)-7,7- dimethyl-1-(2,2,2-trifluoroethyl)- 4,5,6,7-tetrahydro-1H-indazole-3- carboxamide |
| V99A | 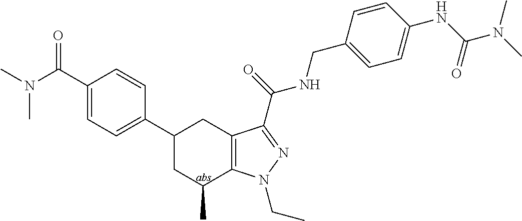 | (7S)-5-(4- (dimethylcarbamoyl)phenyl)-N-(4- (3,3-dimethylureido)benzyl)-1- ethyl-7-methyl- 4,5,6,7-tetrahydro-1H-indazole-3- carboxamide |
| V100A | 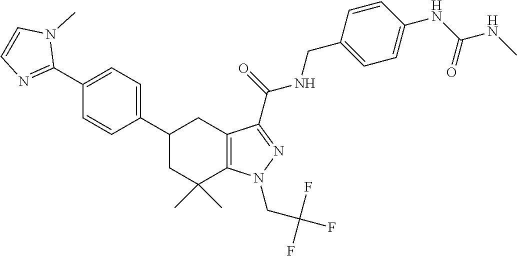 [SMILES: CNC(=O)Nc1ccc(CNC(=O)c2nn(CC(F)(F)F)c3c2CC(c2ccc(-c4nccn4C)cc2)CC3(C)C)cc1] | 7,7-dimethyl-5-(4-(1-methyl-1H- imidazol-2-yl)phenyl)-N-(4-(3- methylureido)benzyl)-1-(2,2,2- trifluoroethyl)- 4,5,6,7-tetrahydro-1H-indazole-3- carboxamide |
| V101A | 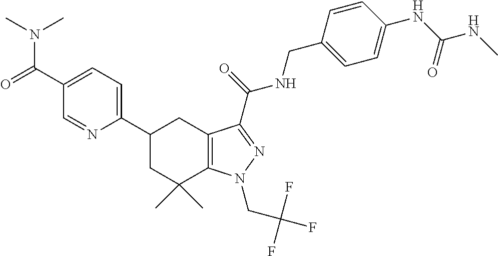 [SMILES: CNC(=O)Nc1ccc(CNC(=O)c2nn(CC(F)(F)F)c3c2CC(c2ccc(C(=O)N(C)C)cn2)CC3(C)C)cc1] | 5-(5-(dimethylcarbamoyl)pyridin- 2-yl)-7,7-dimethyl-N-(4-(3- methylureido)benzyl)-1-(2,2,2- trifluoroethyl)- 4,5,6,7-tetrahydro-1H-indazole-3- carboxamide |
| V102A | 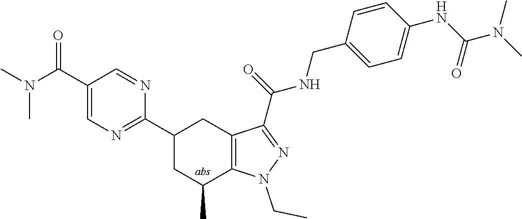 | (7S)-5-(5- (dimethylcarbamoyl)pyrimidin-2- yl)-N-(4-(3,3- dimethylureido)benzyl)-1-ethyl-7- methyl- 4,5,6,7-tetrahydro-1H-indazole-3- carboxamide |
| V103A | 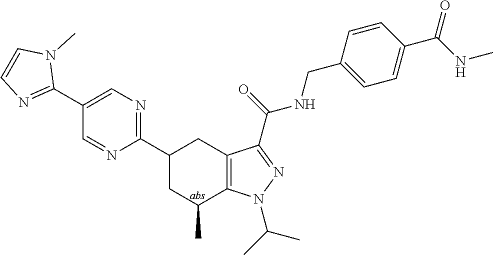 [SMILES: CNC(=O)c1ccc(CNC(=O)c2nn(C(C)C)c3c2CC(c2ncc(-c4nccn4C)cn2)C[C@@H]3C)cc1] | (7S)-1-isopropyl-7-methyl-5-(5-(1- methyl-1H-imidazol-2-yl) pyrimidin-2-yl)-N-(4- (methylcarbamoyl)benzyl)- 4,5,6,7-tetrahydro-1H-indazole-3- carboxamide |
| V104A | 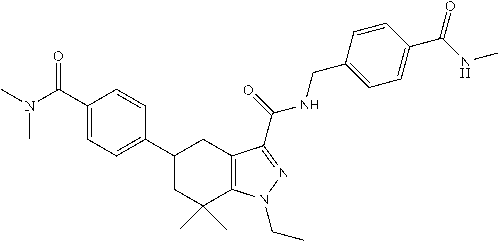 | 5-(4-(dimethylcarbamoyl)phenyl)- 1-ethyl-7,7-dimethyl-N-(4- (methylcarbamoyl)benzyl)- 4,5,6,7-tetrahydro-1H-indazole-3- carboxamide |
| V105A | 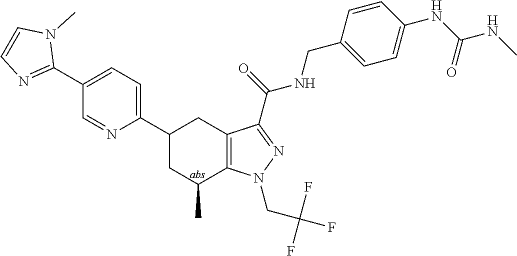 [SMILES: CNC(=O)Nc1ccc(CNC(=O)c2nn(CC(F)(F)F)c3c2CC(c2ccc(-c4nccn4C)cn2)C[C@@H]3C)cc1] | (7S)-7-methyl-5-(5-(1-methyl-1H- imidazol-2-yl)pyridin-2-yl)-N-(4- (3-methylureido)benzyl)-1- (2,2,2-trifluoroethyl)- 4,5,6,7-tetrahydro-1H-indazole-3- carboxamide |
| V106A | 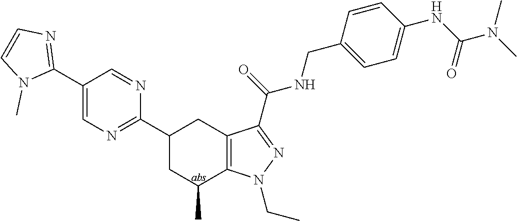 [SMILES: CCn1nc(C(=O)NCc2ccc(NC(=O)N(C)C)cc2)c2c1[C@@H](C)CC(c1ncc(-c3nccn3C)cn1)C2] | (7S)-N-(4-(3,3- dimethylureido)benzyl)-1-ethyl-7- methyl-5-(5-(1-methyl-1H- imidazol-2-yl)pyrimidin-2-yl)- 4,5,6,7-tetrahydro-1H-indazole-3- carboxamide |
| V107A | 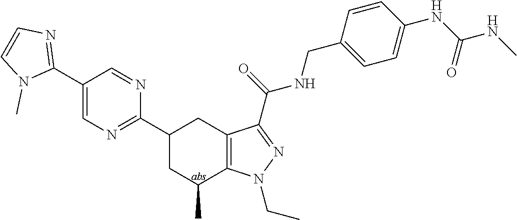 [SMILES: CCn1nc(C(=O)NCc2ccc(NC(=O)NC)cc2)c2c1[C@@H](C)CC(c1ncc(-c3nccn3C)cn1)C2] | (7S)-1-ethyl-7-methyl-5-(5-(1- methyl-1H-imidazol-2-yl) pyrimidin-2-yl)-N-(4-(3- methylureido)benzyl)- 4,5,6,7-tetrahydro-1H-indazole-3- carboxamide |
| V108A | 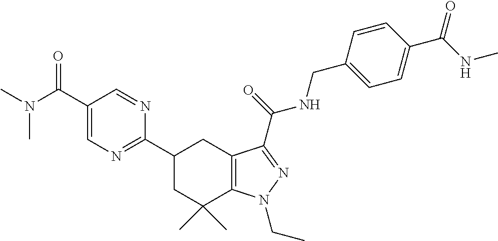 | 5-(5- (dimethylcarbamoyl)pyrimidin-2- yl)-1-ethyl-7,7-dimethyl-N-(4- (methylcarbamoyl)benzyl)- 4,5,6,7-tetrahydro-1H-indazole-3- carboxamide |
| V109A | 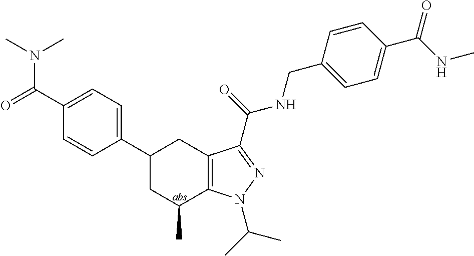 [SMILES: CNC(=O)c1ccc(CNC(=O)c2nn(C(C)C)c3c2CC(c2ccc(C(=O)N(C)C)cc2)C[C@@H]3C)cc1] | (7S)-5-(4- (dimethylcarbamoyl)phenyl)-1- isopropyl-7-methyl-N-(4- (methylcarbamoyl)benzyl)- 4,5,6,7-tetrahydro-1H-indazole-3- carboxamide |
| V110A | 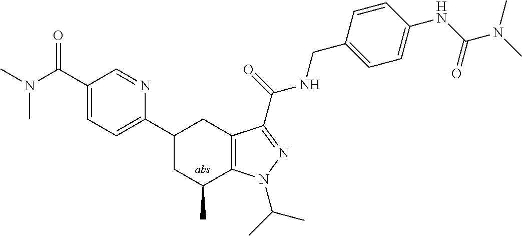 | (7S)-5-(5- (dimethylcarbamoyl)pyridin-2-yl)- N-(4-(3,3-dimethylureido)benzyl)- 1-isopropyl-7-methyl- 4,5,6,7-tetrahydro-1H-indazole-3- carboxamide |
| V111A | 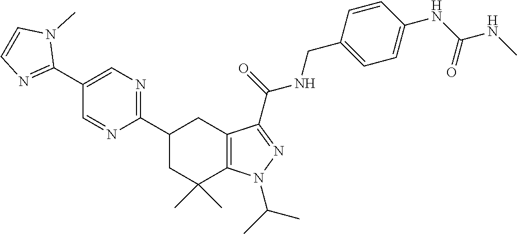 [SMILES: CNC(=O)Nc1ccc(CNC(=O)c2nn(C(C)C)c3c2CC(c2ncc(-c4nccn4C)cn2)CC3(C)C)cc1] | 1-isopropyl-7,7-dimethyl-5-(5-(1- methyl-1H-imidazol-2-yl) pyrimidin-2-yl)-N-(4-(3- methylureido)benzyl)- 4,5,6,7-tetrahydro-1H-indazole-3- carboxamide |
| V112A | 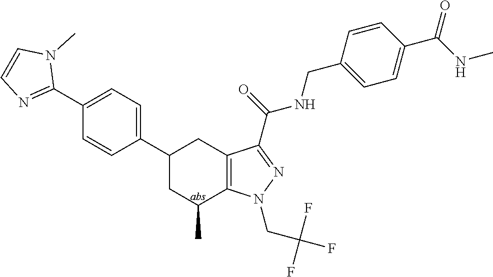 [SMILES: CNC(=O)c1ccc(CNC(=O)c2nn(CC(F)(F)F)c3c2CC(c2ccc(-c4nccn4C)cc2)C[C@@H]3C)cc1] | (7S)-7-methyl-5-(4-(1-methyl-1H- imidazol-2-yl)phenyl)-N-(4- (methylcarbamoyl)benzyl)-1- (2,2,2-trifluoroethyl)- 4,5,6,7-tetrahydro-1H-indazole-3- carboxamide |
| V113A | 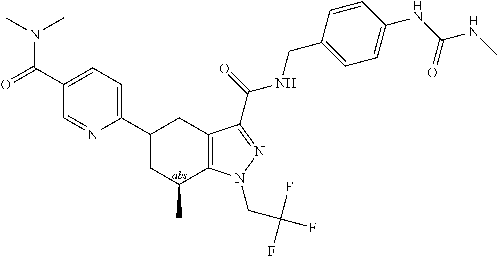 [SMILES: CNC(=O)Nc1ccc(CNC(=O)c2nn(CC(F)(F)F)c3c2CC(c2ccc(C(=O)N(C)C)cn2)C[C@@H]3C)cc1] | (7S)-5-(5- (dimethylcarbamoyl)pyridin-2-yl)- 7-methyl-N-(4-(3- methylureido)benzyl)-1-(2,2,2- trifluoroethyl)- 4,5,6,7-tetrahydro-1H-indazole-3- carboxamide |
| V114A | 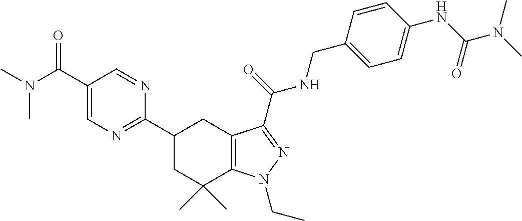 | 5-(5- (dimethylcarbamoyl)pyrimidin-2- yl)-N-(4-(3,3- dimethylureido)benzyl)-1-ethyl- 7,7-dimethyl- 4,5,6,7-tetrahydro-1H-indazole-3- carboxamide |
| V115A | 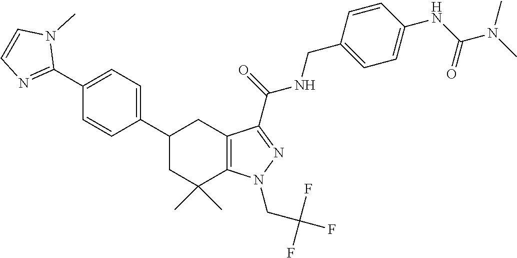 [SMILES: CN(C)C(=O)Nc1ccc(CNC(=O)c2nn(CC(F)(F)F)c3c2CC(c2ccc(-c4nccn4C)cc2)CC3(C)C)cc1] | N-(4-(3,3-dimethylureido)benzyl)- 7,7-dimethyl-5-(4-(1-methyl-1H- imidazol-2-yl)phenyl)-1-(2,2,2- trifluoroethyl)- 4,5,6,7-tetrahydro-1H-indazole-3- carboxamide |
| V116A | 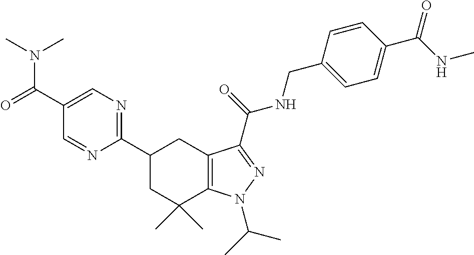 [SMILES: CNC(=O)c1ccc(CNC(=O)c2nn(C(C)C)c3c2CC(c2ncc(C(=O)N(C)C)cn2)CC3(C)C)cc1] | 5-(5- (dimethylcarbamoyl)pyrimidin-2- yl)-1-isopropyl-7,7-dimethyl-N-(4- (methylcarbamoyl)benzyl)- 4,5,6,7-tetrahydro-1H-indazole-3- carboxamide |
| V117A | 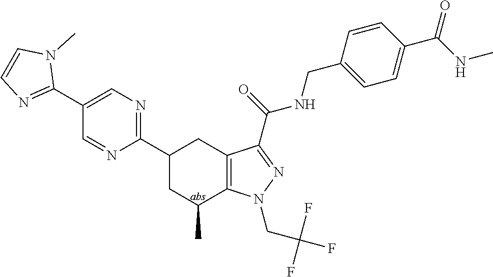 [SMILES: CNC(=O)c1ccc(CNC(=O)c2nn(CC(F)(F)F)c3c2CC(c2ncc(-c4nccn4C)cn2)C[C@@H]3C)cc1] | (7S)-7-methyl-5-(5-(1-methyl-1H- imidazol-2-yl)pyrimidin-2-yl)-N-(4- (methylcarbamoyl)benzyl)-1- (2,2,2-trifluoroethyl)- 4,5,6,7-tetrahydro-1H-indazole-3- carboxamide |
| V118A | 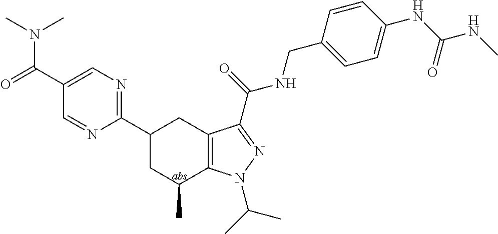 [SMILES: CNC(=O)Nc1ccc(CNC(=O)c2nn(C(C)C)c3c2CC(c2ncc(C(=O)N(C)C)cn2)C[C@@H]3C)cc1] | (7S)-5-(5- (dimethylcarbamoyl)pyrimidin-2- yl)-1-isopropyl-7-methyl-N-(4-(3- methylureido)benzyl)- 4,5,6,7-tetrahydro-1H-indazole-3- carboxamide |
| V119A | 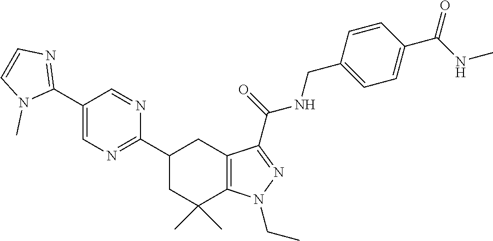 [SMILES: CCn1nc(C(=O)NCc2ccc(C(=O)NC)cc2)c2c1C(C)(C)CC(c1ncc(-c3nccn3C)cn1)C2] | 1-ethyl-7,7-dimethyl-5-(5-(1- methyl-1H-imidazol-2-yl) pyrimidin-2-yl)-N-(4- (methylcarbamoyl)benzyl)- 4,5,6,7-tetrahydro-1H-indazole-3- carboxamide |
| V120A | 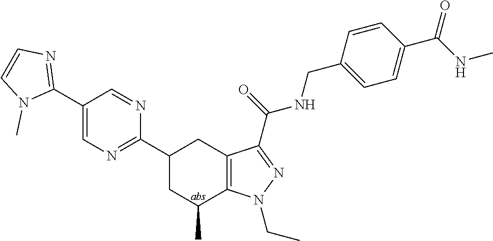 [SMILES: CCn1nc(C(=O)NCc2ccc(C(=O)NC)cc2)c2c1[C@@H](C)CC(c1ncc(-c3nccn3C)cn1)C2] | (7S)-1-ethyl-7-methyl-5-(5-(1- methyl-1H-imidazol-2-yl) pyrimidin-2-yl)-N-(4- (methylcarbamoyl)benzyl)- 4,5,6,7-tetrahydro-1H-indazole-3- carboxamide |
| V121A | 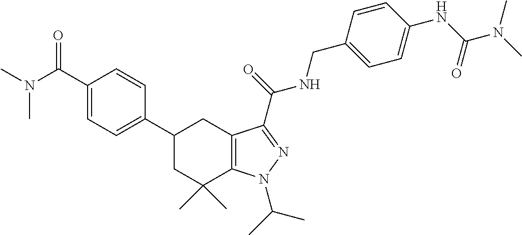 | 5-(4-(dimethylcarbamoyl)phenyl)- N-(4-(3,3-dimethylureido)benzyl)- 1-isopropyl-7,7-dimethyl- 4,5,6,7-tetrahydro-1H-indazole-3- carboxamide |
| V122A | 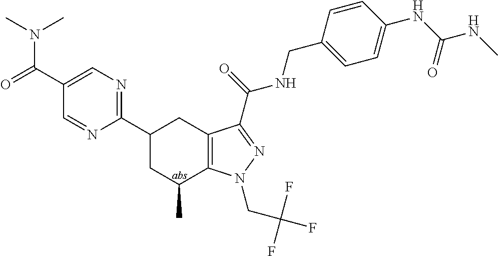 [SMILES: CNC(=O)Nc1ccc(CNC(=O)c2nn(CC(F)(F)F)c3c2CC(c2ncc(C(=O)N(C)C)cn2)C[C@@H]3C)cc1] | (7S)-5-(5- (dimethylcarbamoyl)pyrimidin-2- yl)-7-methyl-N-(4-(3- methylureido)benzyl)-1-(2,2,2- trifluoroethyl)- 4,5,6,7-tetrahydro-1H-indazole-3- carboxamide |
| V123A | 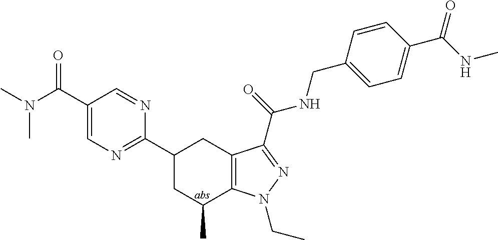 | (7S)-5-(5- (dimethylcarbamoyl)pyrimidin-2- yl)-1-ethyl-7-methyl-N-(4- (methylcarbamoyl)benzyl)- 4,5,6,7-tetrahydro-1H-indazole-3- carboxamide |
| V124A | 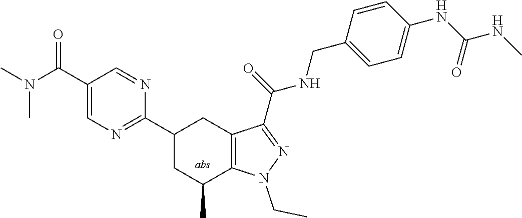 | (7S)-5-(5- (dimethylcarbamoyl)pyrimidin-2- yl)-1-ethyl-7-methyl-N-(4-(3- methylureido)benzyl)- 4,5,6,7-tetrahydro-1H-indazole-3- carboxamide |
| V125A | 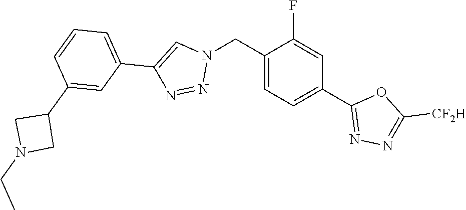 [SMILES: CCN1CC(c2cccc(-c3cn(Cc4ccc(-c5nnc(C(F)F)o5)cc4F)nn3)c2)C1] | (7S)-1-ethyl-7-methyl-5-(4-(1- methyl-1H-imidazol-2-yl)phenyl)- N-(4-(methylcarbamoyl)benzyl)- 4,5,6,7-tetrahydro-1H-indazole-3- carboxamide |
| V126A | 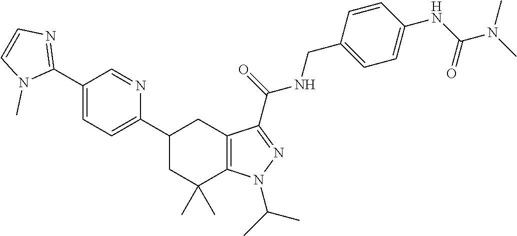 [SMILES: CC(C)n1nc(C(=O)NCc2ccc(NC(=O)N(C)C)cc2)c2c1C(C)(C)CC(c1ccc(-c3nccn3C)cn1)C2] | N-(4-(3,3-dimethylureido)benzyl)- 1-isopropyl-7,7-dimethyl-5-(5-(1- methyl-1H-imidazol-2-yl)pyridin-2- yl)- 4,5,6,7-tetrahydro-1H-indazole-3- carboxamide |
| V127A | 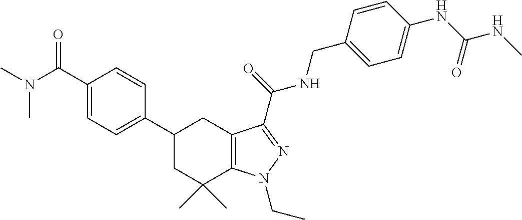 | 5-(4-(dimethylcarbamoyl)phenyl)- 1-ethyl-7,7-dimethyl-N-(4-(3- methylureido)benzyl)- 4,5,6,7-tetrahydro-1H-indazole-3- carboxamide |
| V128A | 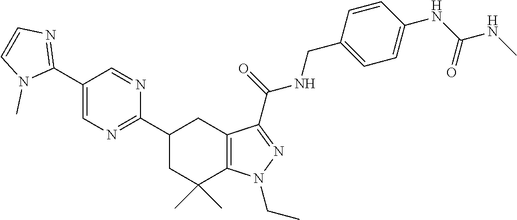 [SMILES: CCn1nc(C(=O)NCc2ccc(NC(=O)NC)cc2)c2c1C(C)(C)CC(c1ncc(-c3nccn3C)cn1)C2] | 1-ethyl-7,7-dimethyl-5-(5-(1- methyl-1H-imidazol-2-yl) pyrimidin-2-yl)-N-(4-(3- methylureido)benzyl)- 4,5,6,7-tetrahydro-1H-indazole-3- carboxamide |
| V129A | 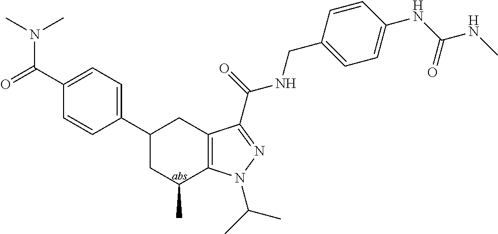 [SMILES: CNC(=O)Nc1ccc(CNC(=O)c2nn(C(C)C)c3c2CC(c2ccc(C(=O)N(C)C)cc2)C[C@@H]3C)cc1] | (7S)-5-(4- (dimethylcarbamoyl)phenyl)-1- isopropyl-7-methyl-N-(4-(3- methylureido)benzyl)- 4,5,6,7-tetrahydro-1H-indazole-3- carboxamide |
| V130A | 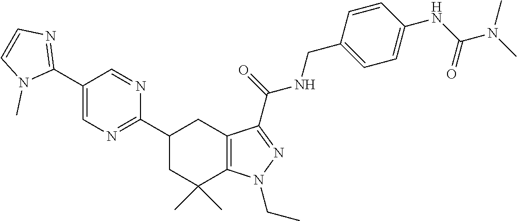 [SMILES: CCn1nc(C(=O)NCc2ccc(NC(=O)N(C)C)cc2)c2c1C(C)(C)CC(c1ncc(-c3nccn3C)cn1)C2] | N-(4-(3,3-dimethylureido)benzyl)- 1-ethyl-7,7-dimethyl-5-(5-(1- methyl-1H-imidazol-2-yl) pyrimidin-2-yl)- 4,5,6,7-tetrahydro-1H-indazole-3- carboxamide |
| V131A | 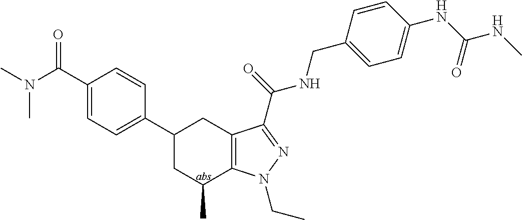 | (7S)-5-(4- (dimethylcarbamoyl)phenyl)-1- ethyl-7-methyl-N-(4-(3- methylureido)benzyl)- 4,5,6,7-tetrahydro-1H-indazole-3- carboxamide |
| V132A | 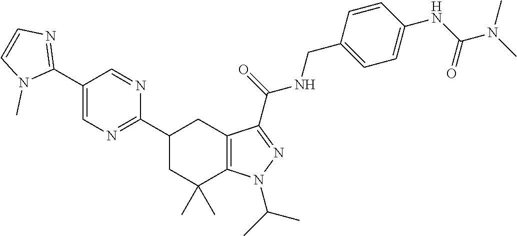 [SMILES: CC(C)n1nc(C(=O)NCc2ccc(NC(=O)N(C)C)cc2)c2c1C(C)(C)CC(c1ncc(-c3nccn3C)cn1)C2] | N-(4-(3,3-dimethylureido)benzyl)- 1-isopropyl-7,7-dimethyl-5-(5-(1- methyl-1H-imidazol-2-yl) pyrimidin-2-yl)- 4,5,6,7-tetrahydro-1H-indazole-3- carboxamide |
| V133A | 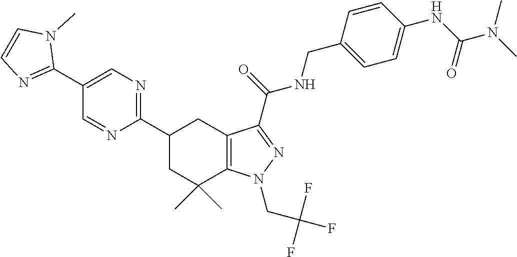 [SMILES: CN(C)C(=O)Nc1ccc(CNC(=O)c2nn(CC(F)(F)F)c3c2CC(c2ncc(-c4nccn4C)cn2)CC3(C)C)cc1] | N-(4-(3,3-dimethylureido)benzyl)- 7,7-dimethyl-5-(5-(1-methyl-1H- imidazol-2-yl)pyrimidin-2-yl)-1- (2,2,2-trifluoroethyl)- 4,5,6,7-tetrahydro-1H-indazole-3- carboxamide |
| V134A | 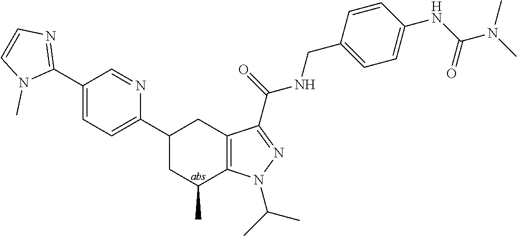 [SMILES: CC(C)n1nc(C(=O)NCc2ccc(NC(=O)N(C)C)cc2)c2c1[C@@H](C)CC(c1ccc(-c3nccn3C)cn1)C2] | (7S)-N-(4-(3,3- dimethylureido)benzyl)-1- isopropyl-7-methyl-5-(5-(1-methyl- 1H-imidazol-2-yl)pyridin-2-yl)- 4,5,6,7-tetrahydro-1H-indazole-3- carboxamide |
| V135A | 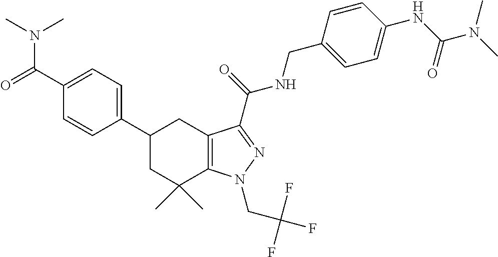 [SMILES: CN(C)C(=O)Nc1ccc(CNC(=O)c2nn(CC(F)(F)F)c3c2CC(c2ccc(C(=O)N(C)C)cc2)CC3(C)C)cc1] | 5-(4-(dimethylcarbamoyl)phenyl)- N-(4-(3,3-dimethylureido)benzyl)- 7,7-dimethyl-1-(2,2,2- trifluoroethyl)- 4,5,6,7-tetrahydro-1H-indazole-3- carboxamide |
| V136A | 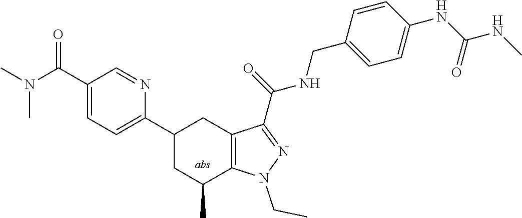 | (7S)-5-(5- (dimethylcarbamoyl)pyridin-2-yl)- 1-ethyl-7-methyl-N-(4-(3- methylureido)benzyl)- 4,5,6,7-tetrahydro-1H-indazole-3- carboxamide |
| V137A | 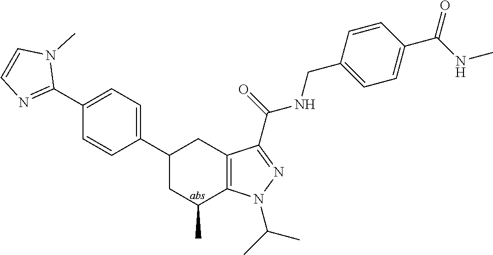 [SMILES: CNC(=O)c1ccc(CNC(=O)c2nn(C(C)C)c3c2CC(c2ccc(-c4nccn4C)cc2)C[C@@H]3C)cc1] | (7S)-1-isopropyl-7-methyl-5-(4-(1- methyl-1H-imidazol-2-yl)phenyl)- N-(4-(methylcarbamoyl)benzyl)- 4,5,6,7-tetrahydro-1H-indazole-3- carboxamide |
| V138A | 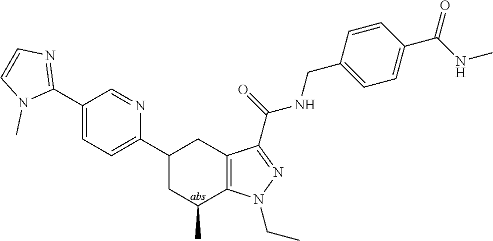 [SMILES: CCn1nc(C(=O)NCc2ccc(C(=O)NC)cc2)c2c1[C@@H](C)CC(c1ccc(-c3nccn3C)cn1)C2] | (7S)-1-ethyl-7-methyl-5-(5-(1- methyl-1H-imidazol-2-yl)pyridin- 2-yl)-N-(4- (methylcarbamoyl)benzyl)- 4,5,6,7-tetrahydro-1H-indazole-3- carboxamide |
| V139A | 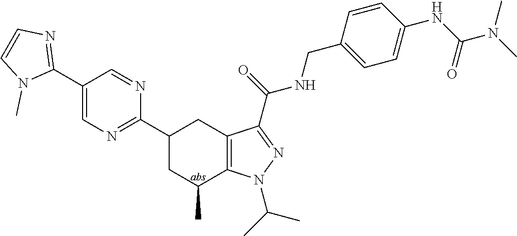 [SMILES: CC(C)n1nc(C(=O)NCc2ccc(NC(=O)N(C)C)cc2)c2c1[C@@H](C)CC(c1ncc(-c3nccn3C)cn1)C2] | (7S)-N-(4-(3,3- dimethylureido)benzyl)-1- isopropyl-7-methyl-5-(5-(1- methyl-1H-imidazol-2-yl) pyrimidin-2-yl)- 4,5,6,7-tetrahydro-1H-indazole-3- carboxamide |
| V140A | 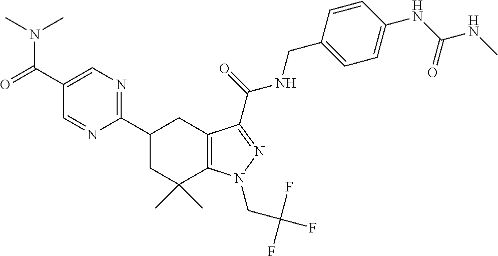 [SMILES: CNC(=O)Nc1ccc(CNC(=O)c2nn(CC(F)(F)F)c3c2CC(c2ncc(C(=O)N(C)C)cn2)CC3(C)C)cc1] | 5-(5- (dimethylcarbamoyl)pyrimidin-2- yl)-7,7-dimethyl-N-(4-(3- methylureido)benzyl)-1-(2,2,2- trifluoroethyl)- 4,5,6,7-tetrahydro-1H-indazole-3- carboxamide |
| V141A | 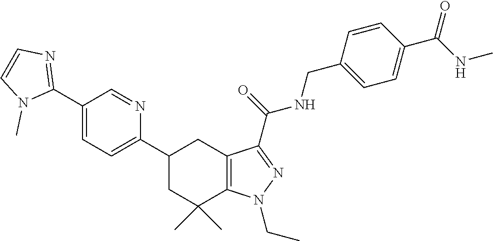 [SMILES: CCn1nc(C(=O)NCc2ccc(C(=O)NC)cc2)c2c1C(C)(C)CC(c1ccc(-c3nccn3C)cn1)C2] | 1-ethyl-7,7-dimethyl-5-(5-(1- methyl-1H-imidazol-2-yl)pyridin-2- yl)-N-(4- (methylcarbamoyl)benzyl)- 4,5,6,7-tetrahydro-1H-indazole-3- carboxamide |
| V142A | 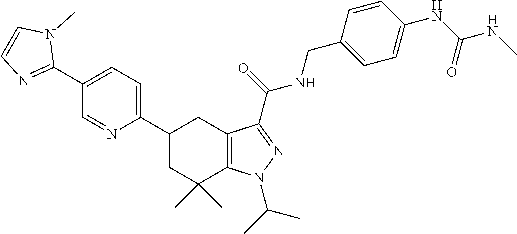 [SMILES: CNC(=O)Nc1ccc(CNC(=O)c2nn(C(C)C)c3c2CC(c2ccc(-c4nccn4C)cn2)CC3(C)C)cc1] | 1-isopropyl-7,7-dimethyl-5-(5-(1- methyl-1H-imidazol-2-yl)pyridin-2- yl)-N-(4-(3-methylureido)benzyl)- 4,5,6,7-tetrahydro-1H-indazole-3- carboxamide |
| V143A | 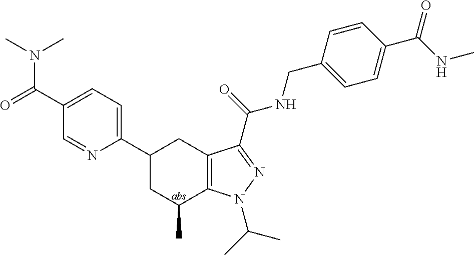 [SMILES: CNC(=O)c1ccc(CNC(=O)c2nn(C(C)C)c3c2CC(c2ccc(C(=O)N(C)C)cn2)C[C@@H]3C)cc1] | (7S)-5-(5- (dimethylcarbamoyl)pyridin-2-yl)- 1-isopropyl-7-methyl-N-(4- (methylcarbamoyl)benzyl)- 4,5,6,7-tetrahydro-1H-indazole-3- carboxamide |
| V144A | 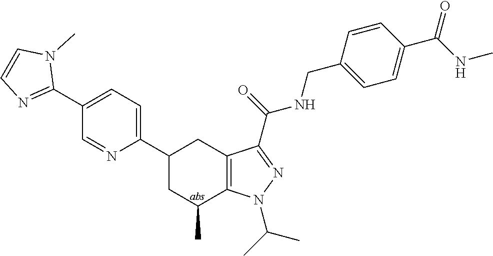 [SMILES: CNC(=O)c1ccc(CNC(=O)c2nn(C(C)C)c3c2CC(c2ccc(-c4nccn4C)cn2)C[C@@H]3C)cc1] | (7S)-1-isopropyl-7-methyl-5-(5-(1- methyl-1H-imidazol-2-yl)pyridin- 2-yl)-N-(4- (methylcarbamoyl)benzyl)- 4,5,6,7-tetrahydro-1H-indazole-3- carboxamide |
| V145A | 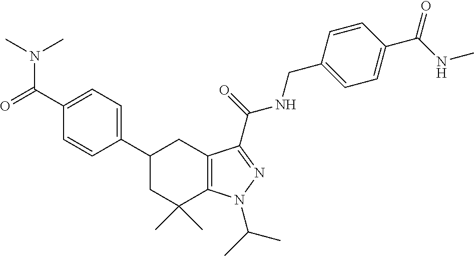 [SMILES: CNC(=O)c1ccc(CNC(=O)c2nn(C(C)C)c3c2CC(c2ccc(C(=O)N(C)C)cc2)CC3(C)C)cc1] | 5-(4-(dimethylcarbamoyl)phenyl)- 1-isopropyl-7,7-dimethyl-N-(4- (methylcarbamoyl)benzyl)- 4,5,6,7-tetrahydro-1H-indazole-3- carboxamide |
| V146A | 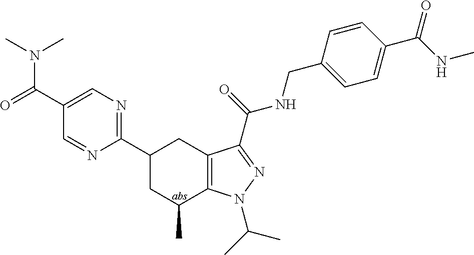 [SMILES: CNC(=O)c1ccc(CNC(=O)c2nn(C(C)C)c3c2CC(c2ncc(C(=O)N(C)C)cn2)C[C@@H]3C)cc1] | (7S)-5-(5- (dimethylcarbamoyl)pyrimidin-2- yl)-1-isopropyl-7-methyl-N-(4- (methylcarbamoyl)benzyl)- 4,5,6,7-tetrahydro-1H-indazole-3- carboxamide |
| V147A | 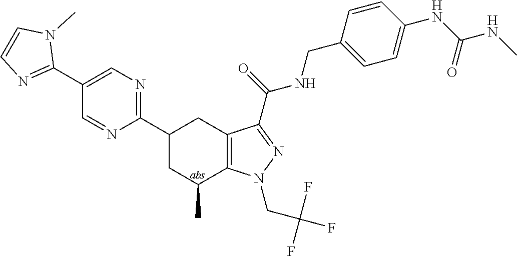 [SMILES: CNC(=O)Nc1ccc(CNC(=O)c2nn(CC(F)(F)F)c3c2CC(c2ncc(-c4nccn4C)cn2)C[C@@H]3C)cc1] | (7S)-7-methyl-5-(5-(1-methyl-1H- imidazol-2-yl)pyrimidin-2-yl)-N- (4-(3-methylureido)benzyl)-1- (2,2,2-trifluoroethyl)- 4,5,6,7-tetrahydro-1H-indazole-3- carboxamide |
| V148A | 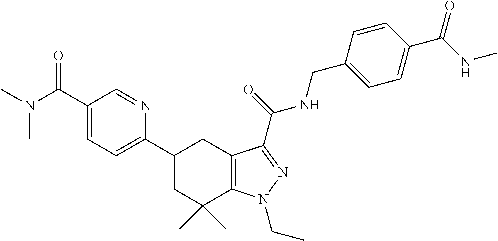 | 5-(5-(dimethylcarbamoyl)pyridin- 2-yl)-1-ethyl-7,7-dimethyl-N-(4- (methylcarbamoyl)benzyl)- 4,5,6,7-tetrahydro-1H-indazole-3- carboxamide |
| V149A | 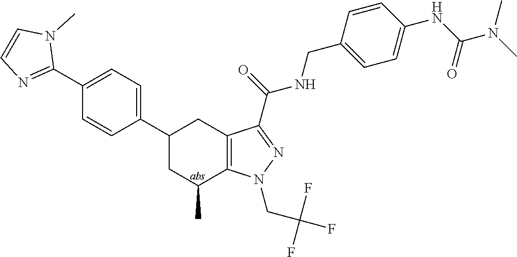 [SMILES: C[C@H]1CC(c2ccc(-c3nccn3C)cc2)Cc2c(C(=O)NCc3ccc(NC(=O)N(C)C)cc3)nn(CC(F)(F)F)c21] | (7S)-N-(4-(3,3- dimethylureido)benzyl)-7-methyl- 5-(4-(1-methyl-1H-imidazol-2- yl)phenyl)-1-(2,2,2-trifluoroethyl)- 4,5,6,7-tetrahydro-1H-indazole-3- carboxamide |
| V150A | 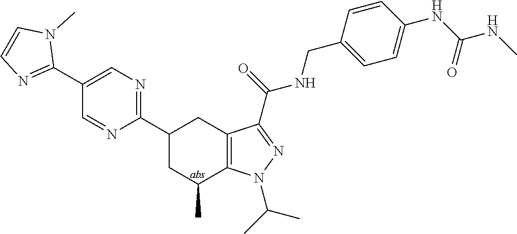 [SMILES: CNC(=O)Nc1ccc(CNC(=O)c2nn(C(C)C)c3c2CC(c2ncc(-c4nccn4C)cn2)C[C@@H]3C)cc1] | (7S)-1-isopropyl-7-methyl-5-(5-(1- methyl-1H-imidazol-2-yl) pyrimidin-2-yl)-N-(4-(3- methylureido)benzyl)- 4,5,6,7-tetrahydro-1H-indazole-3- carboxamide |
| V151A | 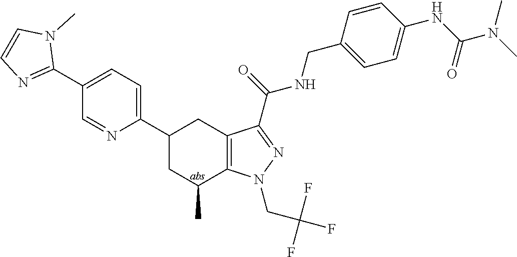 [SMILES: C[C@H]1CC(c2ccc(-c3nccn3C)cn2)Cc2c(C(=O)NCc3ccc(NC(=O)N(C)C)cc3)nn(CC(F)(F)F)c21] | (7S)-N-(4-(3,3- dimethylureido)benzyl)-7-methyl- 5-(5-(1-methyl-1H-imidazol-2- yl)pyridin-2-yl)-1-(2,2,2- trifluoroethyl)- 4,5,6,7-tetrahydro-1H-indazole-3- carboxamide |
| V152A | 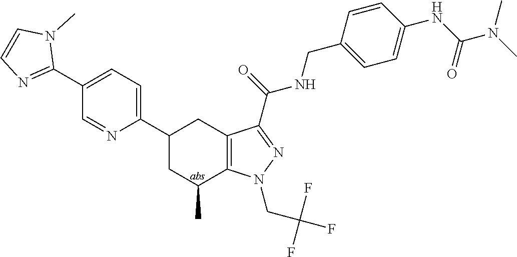 [SMILES: C[C@H]1CC(c2ccc(-c3nccn3C)cn2)Cc2c(C(=O)NCc3ccc(NC(=O)N(C)C)cc3)nn(CC(F)(F)F)c21] | 5-(4-(dimethylcarbamoyl)phenyl)- 1-isopropyl-7,7-dimethyl-N-(4-(3- methylureido)benzyl)- 4,5,6,7-tetrahydro-1H-indazole-3- carboxamide |
| V153A | 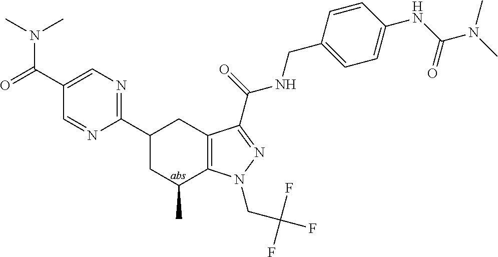 [SMILES: C[C@H]1CC(c2ncc(C(=O)N(C)C)cn2)Cc2c(C(=O)NCc3ccc(NC(=O)N(C)C)cc3)nn(CC(F)(F)F)c21] | (7S)-5-(5- (dimethylcarbamoyl)pyrimidin-2- yl)-N-(4-(3,3- dimethylureido)benzyl)-7-methyl- 1-(2,2,2-trifluoroethyl)- 4,5,6,7-tetrahydro-1H-indazole-3- carboxamide |
| V154A | 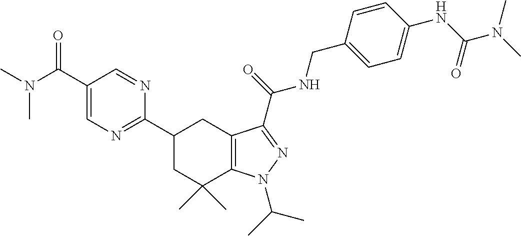 | 5-(5- (dimethylcarbamoyl)pyrimidin-2- yl)-N-(4-(3,3- dimethylureido)benzyl)-1- isopropyl-7,7-dimethyl- 4,5,6,7-tetrahydro-1H-indazole-3- carboxamide |
| V155A | 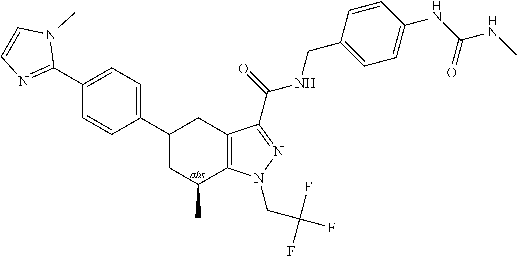 [SMILES: CNC(=O)Nc1ccc(CNC(=O)c2nn(CC(F)(F)F)c3c2CC(c2ccc(-c4nccn4C)cc2)C[C@@H]3C)cc1] | (7S)-7-methyl-5-(4-(1-methyl-1H- imidazol-2-yl)phenyl)-N-(4-(3- methylureido)benzyl)-1-(2,2,2- trifluoroethyl)- 4,5,6,7-tetrahydro-1H-indazole-3- carboxamide |
| V156A | 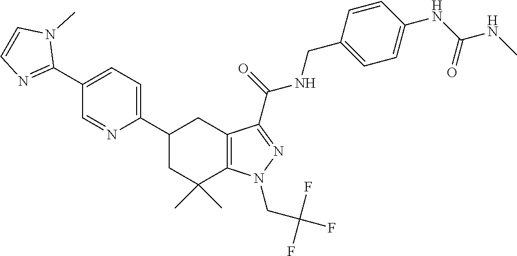 [SMILES: CNC(=O)Nc1ccc(CNC(=O)c2nn(CC(F)(F)F)c3c2CC(c2ccc(-c4nccn4C)cn2)CC3(C)C)cc1] | 7,7-dimethyl-5-(5-(1-methyl-1H- imidazol-2-yl)pyridin-2-yl)-N- (4-(3-methylureido)benzyl)-1- (2,2,2-trifluoroethyl)- 4,5,6,7-tetrahydro-1H-indazole-3- carboxamide |
| V157A | 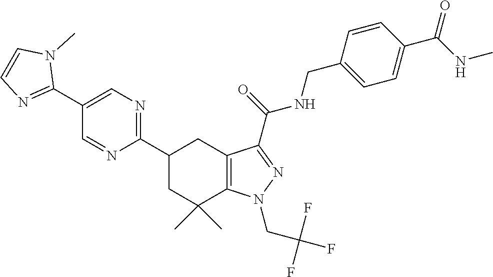 [SMILES: CNC(=O)c1ccc(CNC(=O)c2nn(CC(F)(F)F)c3c2CC(c2ncc(-c4nccn4C)cn2)CC3(C)C)cc1] | 7,7-dimethyl-5-(5-(1-methyl-1H- imidazol-2-yl)pyrimidin-2-yl)-N- (4-(methylcarbamoyl)benzyl)-1- (2,2,2-trifluoroethyl)- 4,5,6,7-tetrahydro-1H-indazole-3- carboxamide |
| V158A | 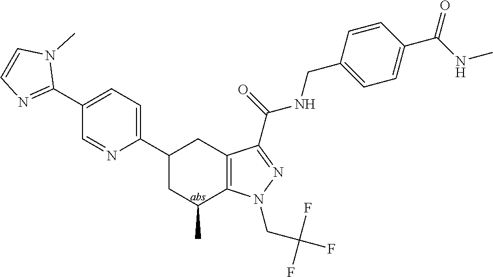 [SMILES: CNC(=O)c1ccc(CNC(=O)c2nn(CC(F)(F)F)c3c2CC(c2ccc(-c4nccn4C)cn2)C[C@@H]3C)cc1] | (7S)-7-methyl-5-(5-(1-methyl-1H- imidazol-2-yl)pyridin-2-yl)-N-(4- (methylcarbamoyl)benzyl)-1- (2,2,2-trifluoroethyl)- 4,5,6,7-tetrahydro-1H-indazole-3- carboxamide |
| V159A | 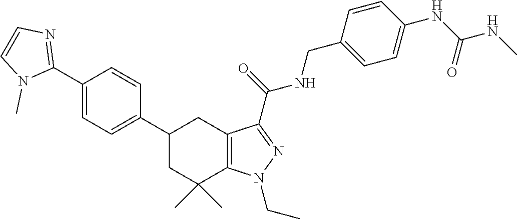 [SMILES: CCn1nc(C(=O)NCc2ccc(NC(=O)NC)cc2)c2c1C(C)(C)CC(c1ccc(-c3nccn3C)cc1)C2] | 1-ethyl-7,7-dimethyl-5-(4-(1- methyl-1H-imidazol-2-yl) phenyl)-N-(4-(3-methylureido) benzyl)- 4,5,6,7-tetrahydro-1H-indazole-3- carboxamide |
| V160A | 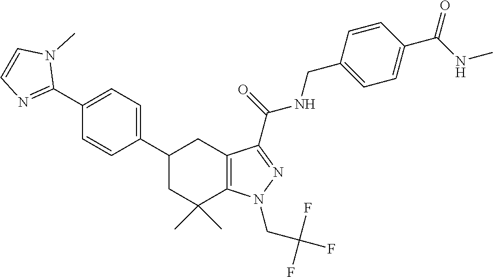 [SMILES: CNC(=O)c1ccc(CNC(=O)c2nn(CC(F)(F)F)c3c2CC(c2ccc(-c4nccn4C)cc2)CC3(C)C)cc1] | 7,7-dimethyl-5-(4-(1-methyl-1H- imidazol-2-yl)phenyl)-N-(4- (methylcarbamoyl)benzyl)-1- (2,2,2-trifluoroethyl)- 4,5,6,7-tetrahydro-1H-indazole-3- carboxamide |
| V161A | 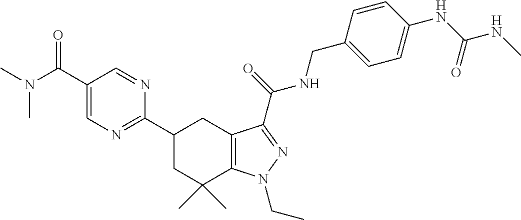 | 5-(5- (dimethylcarbamoyl)pyrimidin-2- yl)-1-ethyl-7,7-dimethyl-N-(4-(3- methylureido)benzyl)- 4,5,6,7-tetrahydro-1H-indazole-3- carboxamide |
| V162A | 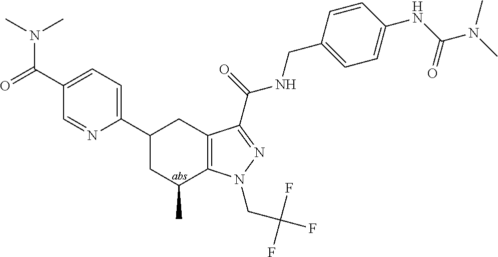 [SMILES: C[C@H]1CC(c2ccc(C(=O)N(C)C)cn2)Cc2c(C(=O)NCc3ccc(NC(=O)N(C)C)cc3)nn(CC(F)(F)F)c21] | (7S)-5-(5- (dimethylcarbamoyl)pyridin-2-yl)- N-(4-(3,3-dimethylureido)benzyl)- 7-methyl-1-(2,2,2-trifluoroethyl)- 4,5,6,7-tetrahydro-1H-indazole-3- carboxamide |
| V163A | 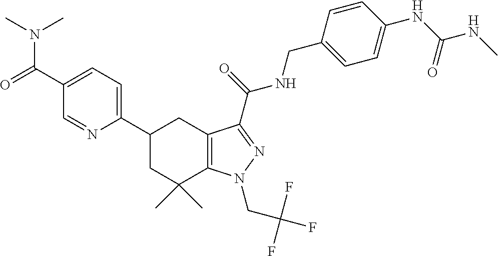 [SMILES: CNC(=O)Nc1ccc(CNC(=O)c2nn(CC(F)(F)F)c3c2CC(c2ccc(C(=O)N(C)C)cn2)CC3(C)C)cc1] | 5-(5-(dimethylcarbamoyl)pyridin- 2-yl)-1-isopropyl-7,7-dimethyl-N- (4-(3-methylureido)benzyl)- 4,5,6,7-tetrahydro-1H-indazole-3- carboxamide |
| V164A | 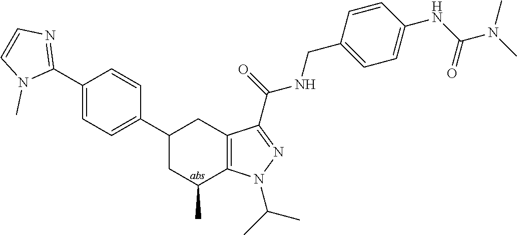 [SMILES: CC(C)n1nc(C(=O)NCc2ccc(NC(=O)N(C)C)cc2)c2c1[C@@H](C)CC(c1ccc(-c3nccn3C)cc1)C2] | (7S)-N-(4-(3,3- dimethylureido)benzyl)-1- isopropyl-7-methyl-5-(4-(1- methyl-1H-imidazol-2-yl)phenyl)- 4,5,6,7-tetrahydro-1H-indazole-3- carboxamide |
| V165A | 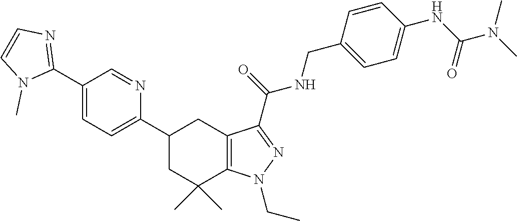 [SMILES: CCn1nc(C(=O)NCc2ccc(NC(=O)N(C)C)cc2)c2c1C(C)(C)CC(c1ccc(-c3nccn3C)cn1)C2] | N-(4-(3,3-dimethylureido)benzyl)- 1-ethyl-7,7-dimethyl-5-(5-(1- methyl-1H-imidazol-2-yl) pyridin-2-yl)-4,5,6,7- tetrahydro-1H-indazole-3- carboxamide |
| V166A | 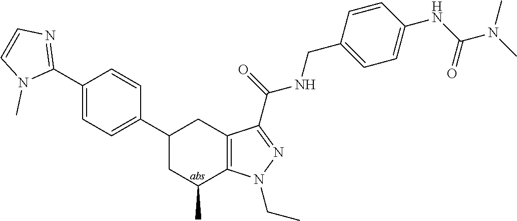 [SMILES: CCn1nc(C(=O)NCc2ccc(NC(=O)N(C)C)cc2)c2c1[C@@H](C)CC(c1ccc(-c3nccn3C)cc1)C2] | (7S)-N-(4-(3,3- dimethylureido)benzyl)-1-ethyl-7- methyl-5-(4-(1-methyl-1H- imidazol-2-yl)phenyl)- 4,5,6,7-tetrahydro-1H-indazole-3- carboxamide |
| V167A | 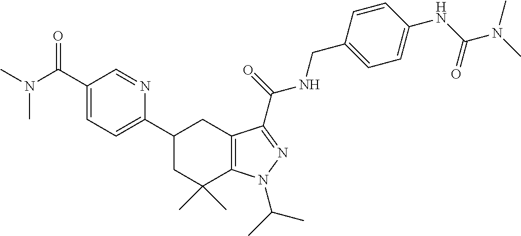 | 5-(5-(dimethylcarbamoyl)pyridin- 2-yl)-N-(4-(3,3- dimethylureido)benzyl)-1- isopropyl-7,7-dimethyl- 4,5,6,7-tetrahydro-1H-indazole-3- carboxamide |
| V168A | 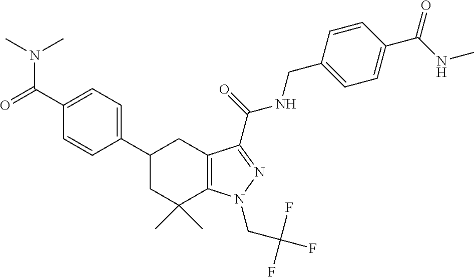 [SMILES: CNC(=O)c1ccc(CNC(=O)c2nn(CC(F)(F)F)c3c2CC(c2ccc(C(=O)N(C)C)cc2)CC3(C)C)cc1] | 5-(4-(dimethylcarbamoyl)phenyl)- 7,7-dimethyl-N-(4- (methylcarbamoyl)benzyl)-1- (2,2,2-trifluoroethyl)- 4,5,6,7-tetrahydro-1H-indazole-3- carboxamide |
| V169A | 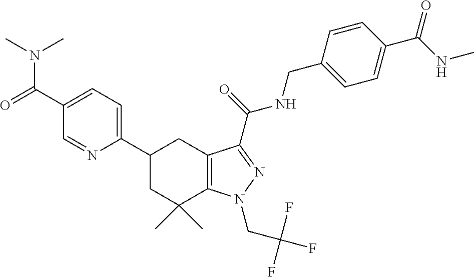 [SMILES: CNC(=O)c1ccc(CNC(=O)c2nn(CC(F)(F)F)c3c2CC(c2ccc(C(=O)N(C)C)cn2)CC3(C)C)cc1] | 5-(5-(dimethylcarbamoyl)pyridin- 2-yl)-7,7-dimethyl-N-(4- (methylcarbamoyl)benzyl)-1- (2,2,2-trifluoroethyl)- 4,5,6,7-tetrahydro-1H-indazole-3- carboxamide |
| V170A | 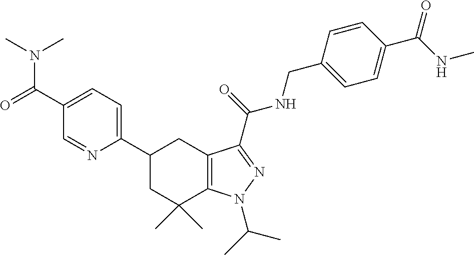 [SMILES: CNC(=O)c1ccc(CNC(=O)c2nn(C(C)C)c3c2CC(c2ccc(C(=O)N(C)C)cn2)CC3(C)C)cc1] | 5-(5-(dimethylcarbamoyl)pyridin- 2-yl)-1-isopropyl-7,7-dimethyl-N- (4-(methylcarbamoyl)benzyl)- 4,5,6,7-tetrahydro-1H-indazole-3- carboxamide |
| V171A | 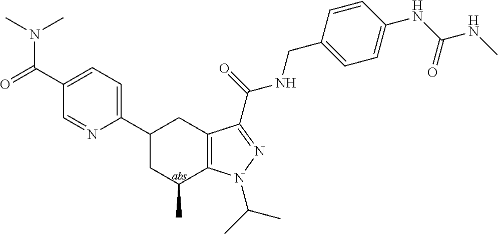 [SMILES: CNC(=O)Nc1ccc(CNC(=O)c2nn(C(C)C)c3c2CC(c2ccc(C(=O)N(C)C)cn2)C[C@@H]3C)cc1] | (7S)-5-(5- (dimethylcarbamoyl)pyridin-2-yl)- 1-isopropyl-7-methyl-N-(4-(3- methylureido)benzyl)- 4,5,6,7-tetrahydro-1H-indazole-3- carboxamide |
| V172A | 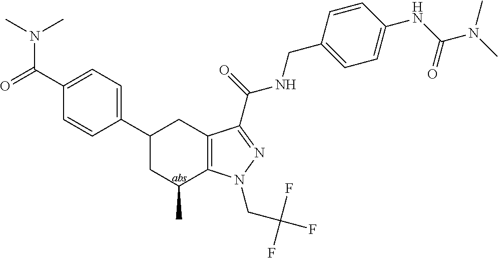 [SMILES: C[C@H]1CC(c2ccc(C(=O)N(C)C)cc2)Cc2c(C(=O)NCc3ccc(NC(=O)N(C)C)cc3)nn(CC(F)(F)F)c21] | (7S)-5-(4- (dimethylcarbamoyl)phenyl)-N-(4- (3,3-dimethylureido)benzyl)-7- methyl-1-(2,2,2-trifluoroethyl)- 4,5,6,7-tetrahydro-1H-indazole-3- carboxamide |
| V173A | 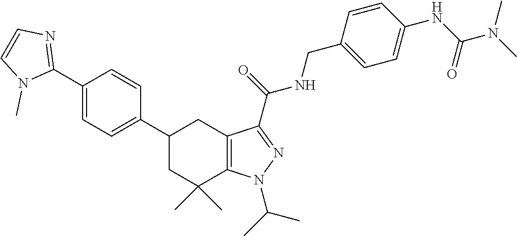 [SMILES: CC(C)n1nc(C(=O)NCc2ccc(NC(=O)N(C)C)cc2)c2c1C(C)(C)CC(c1ccc(-c3nccn3C)cc1)C2] | N-(4-(3,3-dimethylureido)benzyl)- 1-isopropyl-7,7-dimethyl-5-(4-(1- methyl-1H-imidazol-2-yl)phenyl)- 4,5,6,7-tetrahydro-1H-indazole-3- carboxamide |
| V174A | 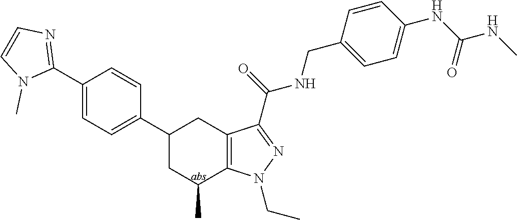 [SMILES: CCn1nc(C(=O)NCc2ccc(NC(=O)NC)cc2)c2c1[C@@H](C)CC(c1ccc(-c3nccn3C)cc1)C2] | (7S)-1-ethyl-7-methyl-5-(4-(1- methyl-1H-imidazol-2-yl)phenyl)- N-(4-(3-methylureido)benzyl)- 4,5,6,7-tetrahydro-1H-indazole-3- carboxamide |
| V175A | 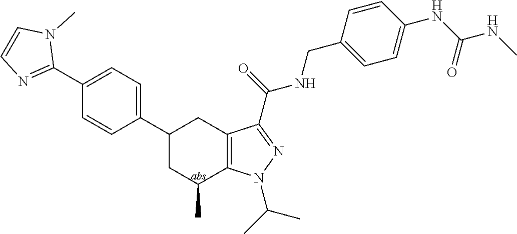 [SMILES: CNC(=O)Nc1ccc(CNC(=O)c2nn(C(C)C)c3c2CC(c2ccc(-c4nccn4C)cc2)C[C@@H]3C)cc1] | (7S)-1-isopropyl-7-methyl-5-(4-(1- methyl-1H-imidazol-2-yl)phenyl)- N-(4-(3-methylureido)benzyl)- 4,5,6,7-tetrahydro-1H-indazole-3- carboxamide |
| V176A | 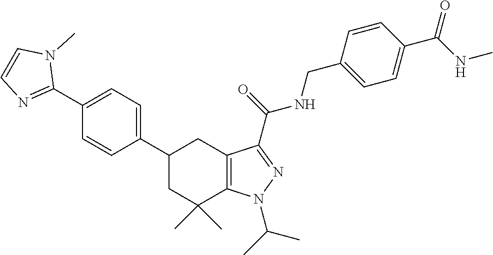 [SMILES: CNC(=O)c1ccc(CNC(=O)c2nn(C(C)C)c3c2CC(c2ccc(-c4nccn4C)cc2)CC3(C)C)cc1] | 1-isopropyl-7,7-dimethyl-5-(4-(1- methyl-1H-imidazol-2-yl)phenyl)- N-(4-(methylcarbamoyl)benzyl)- 4,5,6,7-tetrahydro-1H-indazole-3- carboxamide |
| V177A | 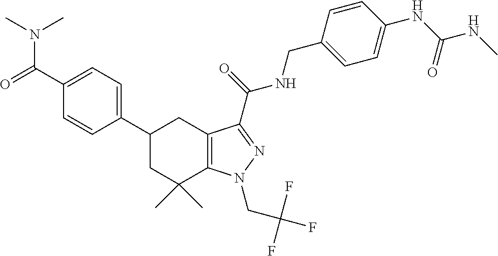 [SMILES: CNC(=O)Nc1ccc(CNC(=O)c2nn(CC(F)(F)F)c3c2CC(c2ccc(C(=O)N(C)C)cc2)CC3(C)C)cc1] | 5-(4-(dimethylcarbamoyl)phenyl)- 7,7-dimethyl-N-(4-(3- methylureido)benzyl)-1-(2,2,2- trifluoroethyl)- 4,5,6,7-tetrahydro-1H-indazole-3- carboxamide |
| V178A | 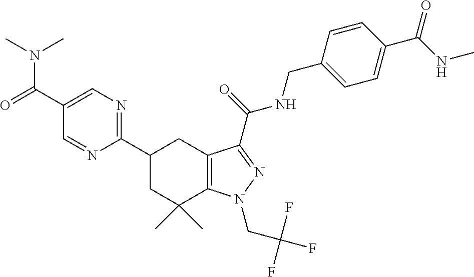 [SMILES: CNC(=O)c1ccc(CNC(=O)c2nn(CC(F)(F)F)c3c2CC(c2ncc(C(=O)N(C)C)cn2)CC3(C)C)cc1] | 5-(5- (dimethylcarbamoyl)pyrimidin-2- yl)-7,7-dimethyl-N-(4- (methylcarbamoyl)benzyl)-1- (2,2,2-trifluoroethyl)- 4,5,6,7-tetrahydro-1H-indazole-3- carboxamide |
| V179A | 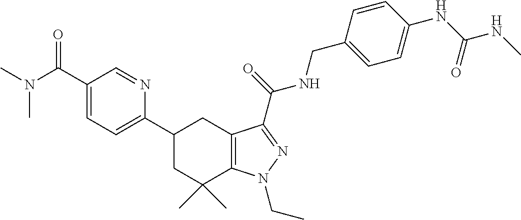 | 5-(5-(dimethylcarbamoyl)pyridin- 2-yl)-1-ethyl-7,7-dimethyl-N-(4-(3- methylureido)benzyl)- 4,5,6,7-tetrahydro-1H-indazole-3- carboxamide |
| V180A | 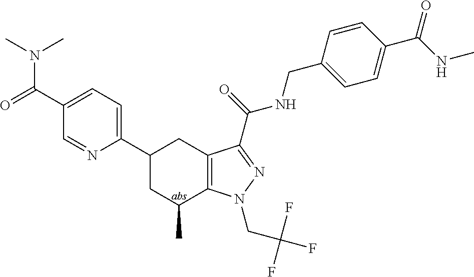 [SMILES: CNC(=O)c1ccc(CNC(=O)c2nn(CC(F)(F)F)c3c2CC(c2ccc(C(=O)N(C)C)cn2)C[C@@H]3C)cc1] | (7S)-5-(5- (dimethylcarbamoyl)pyridin-2-yl)- 7-methyl-N-(4- (methylcarbamoyl)benzyl)-1- (2,2,2-trifluoroethyl)- 4,5,6,7-tetrahydro-1H-indazole-3- carboxamide |
| V181A | 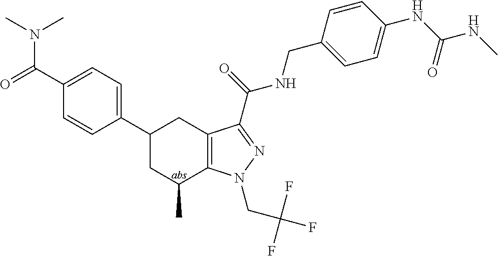 [SMILES: CNC(=O)Nc1ccc(CNC(=O)c2nn(CC(F)(F)F)c3c2CC(c2ccc(C(=O)N(C)C)cc2)C[C@@H]3C)cc1] | (7S)-5-(4- (dimethylcarbamoyl)phenyl)-7- methyl-N-(4-(3- methylureido)benzyl)-1-(2,2,2- trifluoroethyl)- 4,5,6,7-tetrahydro-1H-indazole-3- carboxamide |
| V182A | 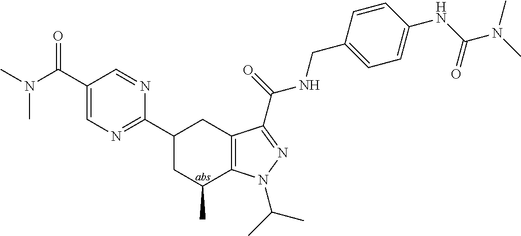 | (7S)-5-(5- (dimethylcarbamoyl)pyrimidin-2- yl)-N-(4-(3,3- dimethylureido)benzyl)-1- isopropyl-7-methyl- 4,5,6,7-tetrahydro-1H-indazole-3- carboxamide |
| V183A | 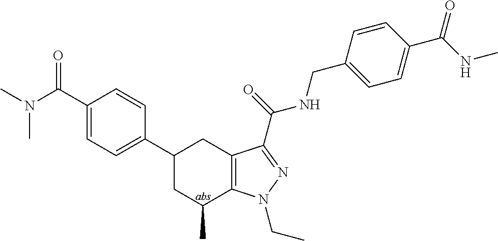 | (7S)-5-(4- (dimethylcarbamoyl)phenyl)-1- ethyl-7-methyl-N-(4- (methylcarbamoyl)benzyl)- 4,5,6,7-tetrahydro-1H-indazole-3- carboxamide |
| V184A | 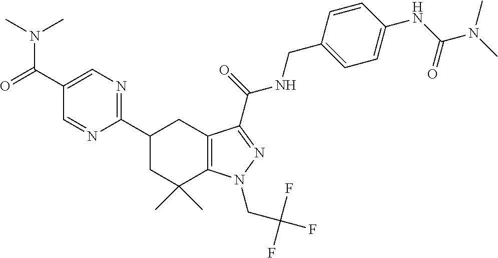 [SMILES: CN(C)C(=O)Nc1ccc(CNC(=O)c2nn(CC(F)(F)F)c3c2CC(c2ncc(C(=O)N(C)C)cn2)CC3(C)C)cc1] | 5-(5- (dimethylcarbamoyl)pyrimidin-2- yl)-N-(4-(3,3- dimethylureido)benzyl)-7,7- dimethyl-1-(2,2,2-trifluoroethyl)- 4,5,6,7-tetrahydro-1H-indazole-3- carboxamide |
| V185A | 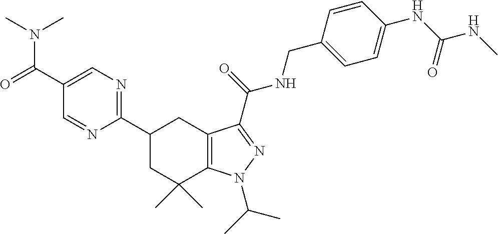 [SMILES: CNC(=O)Nc1ccc(CNC(=O)c2nn(C(C)C)c3c2CC(c2ncc(C(=O)N(C)C)cn2)CC3(C)C)cc1] | 5-(5- (dimethylcarbamoyl)pyrimidin-2- yl)-1-isopropyl-7,7-dimethyl-N-(4- (3-methylureido)benzyl)- 4,5,6,7-tetrahydro-1H-indazole-3- carboxamide |
| V186A | 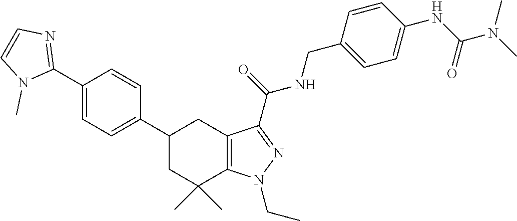 [SMILES: CCn1nc(C(=O)NCc2ccc(NC(=O)N(C)C)cc2)c2c1C(C)(C)CC(c1ccc(-c3nccn3C)cc1)C2] | N-(4-(3,3-dimethylureido)benzyl)- 1-ethyl-7,7-dimethyl-5-(4-(1- methyl-1H-imidazol-2-yl)phenyl)- 4,5,6,7-tetrahydro-1H-indazole-3- carboxamide |
| V187A | 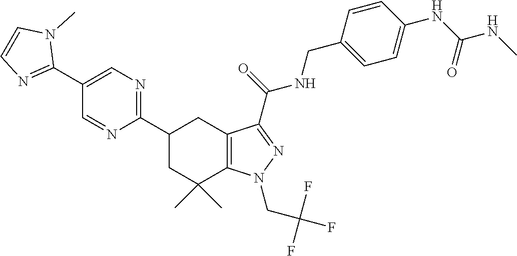 [SMILES: CNC(=O)Nc1ccc(CNC(=O)c2nn(CC(F)(F)F)c3c2CC(c2ncc(-c4nccn4C)cn2)CC3(C)C)cc1] | 7,7-dimethyl-5-(5-(1-methyl-1H- imidazol-2-yl)pyrimidin-2-yl)-N-(4- (3-methylureido)benzyl)-1-(2,2,2- trifluoroethyl)- 4,5,6,7-tetrahydro-1H-indazole-3- carboxamide |

### Preparation F1

### 1-(1-Methyl-6-(1-(2-(methylamino)ethyl)piperidin-4-yl)-1H-indazol-3-yl)dihydropyrimidine-2,4(1H,3H)-dione bis(2,2,2-trifluoroacetate)

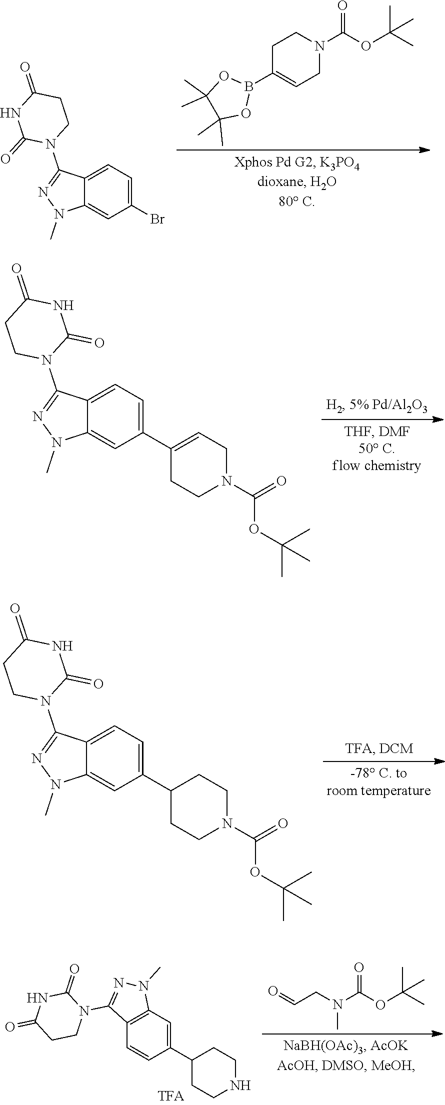 [SMILES: Cn1nc(N2CCC(=O)NC2=O)c2ccc(Br)cc21 ; CC(C)(C)OC(=O)N1CC=C(B2OC(C)(C)C(C)(C)O2)CC1 ; Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21 ; CN(CC=O)C(=O)OC(C)(C)C]

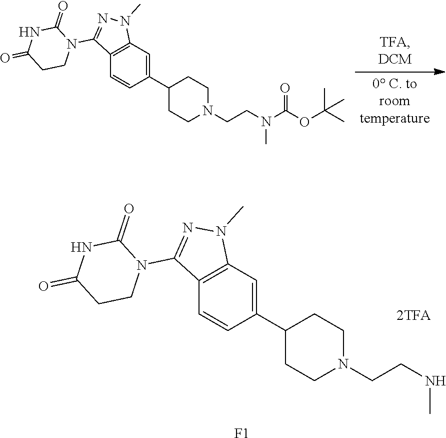 [SMILES: CN(CCN1CCC(c2ccc3c(N4CCC(=O)NC4=O)nn(C)c3c2)CC1)C(=O)OC(C)(C)C ; CNCCN1CCC(c2ccc3c(N4CCC(=O)NC4=O)nn(C)c3c2)CC1 ; "F1"]

Step 1. Preparation of tert-butyl 4-(3-(2,4-dioxotetrahydropyrimidin-1(2H)-yl)-1-methyl-1H-indazol-6-yl)-3,6-dihydropyridine-1(2H)-carboxylate

To a solution of 1-(6-bromo-1-methyl-1H-indazol-3-yl)dihydropyrimidine-2,4(1H,3H)-dione (CAS: 2446913-88-6; 20.0 g, 61.89 mmol) and tert-butyl 4-(4,4,5,5-tetramethyl-1,3,2-dioxaborolan-2-yl)-3,6-dihydropyridine-1(2H)-carboxylate (CAS: 286961-14-6; 20.1 g, 65.0 mmol) in dioxane (334 mL) and H₂O (33 mL) was added K₃PO₄ (26.3 g, 124 mmol) and Xphos Pd G2 catalyst (CAS: 1310584-14-5; 5.8 g, 0.12 mmol). The reaction mixture was degassed with nitrogen gas for 3 minutes then stirred at 80 °C for 2 h. The suspension was filtered then poured into water (300 mL). The mixture was extracted with EtOAc (200 mL×3). The combined organic layer was dried with Na₂SO₄ and concentrated in vacuo. The residue was diluted with DCM (3 mL) and hexane (100 mL) then filtered. The filter cake was washed with hexane (20 mL×2) then concentrated in vacuo to give a brown solid. The solid was purified by column chromatography (silica gel; 100% DCM then 5% MeOH in DCM) to provide the crude as a brown solid. The solid was diluted DCM (20 mL) and hexane (200 mL) then filtered. The filter cake was washed with hexane (50 mL×2) then concentrated in vacuo to provide tert-butyl 4-(3-(2,4-dioxotetrahydropyrimidin-1(2H)-yl)-1-methyl-1H-indazol-6-yl)-3,6-dihydropyridine-1(2H)-carboxylate (21.3 g, 80.9% yield) as a brown solid. (LC/MS) m/z (M+H)⁺=426.2. ¹H NMR (400 MHz, (CD₃)₂SO) δ ppm: 10.55 (s, 1H), 7.63-7.56 (m, 2H), 7.30-7.23 (m, 1H), 6.28 (s, 1H), 4.04 (s, 2H), 3.99 (s, 3H), 3.92 (t, 2H), 3.58 (t, 2H), 2.76 (t, 2H), 2.60-2.52 (m, 2H), 1.44 (s, 9H).

### Step 2. Preparation of tert-butyl 4-(3-(2,4-dioxotetrahydropyrimidin-1(2H)-yl)-1-methyl-1H-indazol-6-yl)piperidine-1-carboxylate

The reaction was carried out via flow chemistry. The same reaction was conducted in two batches then combined for purification. A solution of the first batch was prepared with tert-butyl 4-(3-(2,4-dioxotetrahydropyrimidin-1(2H)-yl)-1-methyl-1H-indazol-6-yl)-3,6-dihydropyridine-1(2H)-carboxylate (21.3 g, 50.1 mmol) in DMF (284 mL) and THF (852 mL), which was pumped with a flow rate of 1.5 mL/min. The solution of the first batch was passed through the fixed bed (9.525(⅜″)mm) packed with granular catalyst 5% Pd/Al₂O₃ (17.2 g) with a 25.0 mL volume at 50 °C. The H₂ back pressure regulator was adjusted to 1 MPa and the H₂ had a rate of 6.1 sccm. The reaction mixture of the first batch was continuously collected from the reactor outlet into a bottle.

The same reaction was conducted with tert-butyl 4-(3-(2,4-dioxotetrahydropyrimidin-1(2H)-yl)-1-methyl-1H-indazol-6-yl)-3,6-dihydropyridine-1(2H)-carboxylate (10.3 g, 24.2 mmol). The two batches were combined then concentrated in vacuo to give a black solid. The solid was triturated with hexane (300 mL) then filtered. The filter cake was collected to provide tert-butyl 4-(3-(2,4-dioxotetrahydropyrimidin-1(2H)-yl)-1-methyl-1H-indazol-6-yl)piperidine-1-carboxylate (32.0 g, crude) as a black solid. The solid was used in the next step without further purification. ¹H NMR (400 MHz, (CD₃)₂SO) δ ppm: 10.54 (s, 1H), 7.55 (d, 1H), 7.46 (s, 1H), 7.03 (dd, 1H), 4.11 (d, 2H), 3.96 (s, 3H), 3.90 (t, 2H), 3.32 (s, 1H), 2.87-2.71 (m, 4H), 1.81 (d, 2H), 1.67-1.52 (m, 2H), 1.42 (s, 9H).

### Step 3. Preparation of 1-(1-methyl-6-(piperidin-4-yl)-1H-indazol-3-yl)dihydropyrimidine-2,4(1H,3H)-dione trifluoroacetate

At −78 °C, to a reaction mixture of tert-butyl 4-(3-(2,4-dioxotetrahydropyrimidin-1(2H)-yl)-1-methyl-1H-indazol-6-yl)piperidine-1-carboxylate (32.0 g, 74.8 mmol) in DCM (542 mL) was added TFA (108 mL). The suspension was stirred at room temperature for 2 h then concentrated in vacuo. The residue was diluted with H₂O (300 mL) and DCM (200 mL), which caused precipitate to form. The suspension was filtered then the filter cake was collected to give the crude as a brown solid. The solid was lyophilized to provide 1-(1-methyl-6-(piperidin-4-yl)-1H-indazol-3-yl)dihydropyrimidine-2,4(1H,3H)-dione trifluoroacetate (28.3 g, crude) as a grey solid. The solid was used directly in the next step without further purification. (LC/MS) m/z (M+H)⁺=328.1. ¹H NMR (400 MHz, (CD₃)₂SO) δ ppm: 10.56 (s, 1H), 8.65-8.61 (m, 1H), 8.37-8.33 (m, 1H), 7.61 (d, 1H), 7.40 (s, 1H), 7.02 (dd, 1H), 3.98 (s, 3H), 3.91 (t, 2H), 3.46-3.38 (m, 2H), 3.12-2.94 (m, 3H), 2.75 (t, 2H), 2.02 (d, 2H), 1.95-1.80 (m, 2H).

### Step 4. Preparation of tert-butyl(2-(4-(3-(2,4-dioxotetrahydropyrimidin-1(2H)-yl)-1-methyl-1H-indazol-6-yl)piperidin-1-yl)ethyl)(methyl)carbamate

The same reaction was conducted in two batches then combined for purification. To a solution of 1-(1-methyl-6-(piperidin-4-yl)-1H-indazol-3-yl)dihydropyrimidine-2,4(1H,3H)-dione trifluoroacetate (1.50 g, 2.70 mmol) and tert-butyl methyl(2-oxoethyl)carbamate (CAS: 123397-72-4; 0.491 g, 2.84 mmol) in DMSO (15 mL) and MeOH (1.5 mL) was added AcOK (0.928 g, 9.45 mmol), AcOH (0.324 g, 5.40 mmol) then NaBH(OAc)₃ (1.14 g, 5.40 mmol) to form the first batch. The reaction mixture of the first batch was degassed with nitrogen gas for 2 min then stirred at room temperature for 2 h.

A second batch of the same reaction was conducted with 1-(1-methyl-6-(piperidin-4-yl)-1H-indazol-3-yl)dihydropyrimidine-2,4(1H,3H)-dione (CAS: 2796349-49-8; 0.100 g, 0.180 mmol). The two batches were combined then poured into water (20 mL) and extracted with DCM (20 mL×3). The combined organic phase was washed with brine (20 mL×2), dried with Na₂SO₄ and concentrated in vacuo to provide tert-butyl(2-(4-(3-(2,4-dioxotetrahydropyrimidin-1(2H)-yl)-1-methyl-1H-indazol-6-yl)piperidin-1-yl)ethyl)(methyl)carbamate (1.40 g, crude) as a white solid. (LC/MS) m/z (M+H)⁺=485.3. ¹H NMR (400 MHz, (CD₃)₂SO) δ 10.56 (s, 1H), 7.61 (d, 1H), 7.41 (s, 1H), 7.04 (d, 1H), 3.98 (s, 3H), 3.91 (t, 2H), 3.72-3.53 (m, 3H), 2.88-2.71 (m, 6H), 2.62-2.54 (m, 5H), 2.09-2.04 (m, 3H), 1.50-1.31 (m, 10H).

### Step 5. Preparation of 1-(1-methyl-6-(1-(2-(methylamino)ethyl)piperidin-4-yl)-1H-indazol-3-yl)dihydropyrimidine-2,4(1H,3H)-dione bis(2,2,2-trifluoroacetate)

At 0-10 °C, to a solution of tert-butyl(2-(4-(3-(2,4-dioxotetrahydropyrimidin-1(2H)-yl)-1-methyl-1H-indazol-6-yl)piperidin-1-yl)ethyl)(methyl)carbamate (1.40 g, 2.89 mmol) in DCM (8 mL) was added TFA (8 mL). The reaction mixture was stirred at room temperature for 1.5 h then concentrated in vacuo. The residue was dissolved in H₂O (20 mL) then lyophilized to provide 1-(1-methyl-6-(1-(2-(methylamino)ethyl)piperidin-4-yl)-1H-indazol-3-yl)dihydropyrimidine-2,4(1H,3H)-dione bis(2,2,2-trifluoroacetate) (1.55 g, crude) as a white solid. The white solid was used directly in the next step without further purification. (LC/MS) m/z (M+H)⁺=385.2.

### Preparation F2

### 1-(1-Methyl-7-(4-(2-(methylamino)ethyl)piperazin-1-yl)-1H-indazol-3-yl)dihydropyrimidine-2,4(1H,3H)-dione bis(2,2,2-trifluoracetate)

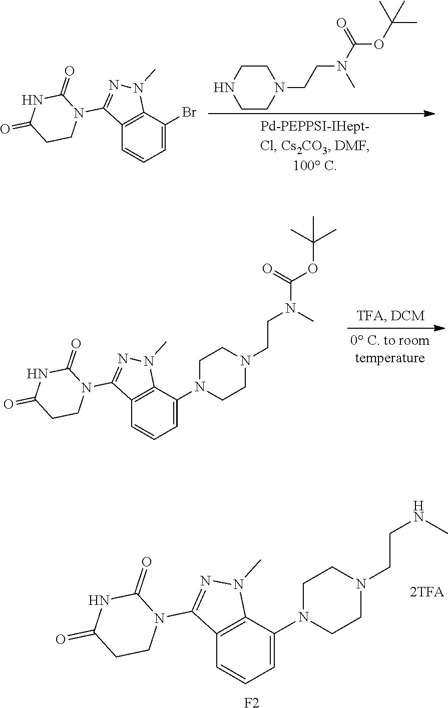

### Step 1. Preparation of tert-butyl(2-(4-(3-(2,4-dioxotetrahydropyrimidin-1(2H)-yl)-1-methyl-1H-indazol-7-yl)piperazin-1-yl)ethyl)(methyl)carbamate

To a solution of 1-(7-bromo-1-methyl-1H-indazol-3-yl)dihydropyrimidine-2,4(1H,3H)-dione (CAS: 3024268-56-9; 0.100 g, 0.309 mmol) and tert-butyl methyl(2-(piperazin-1-yl)ethyl)carbamate (CAS: 1540928-69-5; 79.1 mg, 0.325 mmol) in DMF (1.5 mL) was added Cs₂CO₃ (0.302 g, 0.928 mmol) and Pd-PEPPSI-lHept-CI (CAS: 1814936-54-3; 30.1 mg, 0.0309 mmol) at 100 °C under nitrogen gas. The reaction mixture was stirred at 100 °C for 12 h then filtered. The filtrate was diluted with EtOAc (20 mL) then the organic phase was separated. The organic phase was washed with brine (10 mL×2), dried with Na₂SO₄, then concentrated in vacuo to provide tert-butyl(2-(4-(3-(2,4-dioxotetrahydropyrimidin-1(2H)-yl)-1-methyl-1H-indazol-7-yl)piperazin-1-yl)ethyl)(methyl)carbamate (0.150 g, crude) as a brown gum. The gum was used directly in the next step without further purification.

### Step 2. Preparation of 1-(1-methyl-7-(4-(2-(methylamino)ethyl)piperazin-1-yl)-1H-indazol-3-yl)dihydropyrimidine-2,4(1H,3H)-dione bis(2,2,2-trifluoroacetate)

At 0 °C, to a solution of tert-butyl(2-(4-(3-(2,4-dioxotetrahydropyrimidin-1(2H)-yl)-1-methyl-1H-indazol-7-yl)piperazin-1-yl)ethyl)(methyl)carbamate (0.150 g, 0.309 mmol) in DCM (5 mL) was added TFA (1.0 mL). The reaction mixture was stirred at room temperature for 1.5 h then concentrated in vacuo. The residue was diluted with water then lyophilized to provide 1-(1-methyl-7-(4-(2-(methylamino)ethyl)piperazin-1-yl)-1H-indazol-3-yl)dihydropyrimidine-2,4(1H,3H)-dione bis(2,2,2-trifluoroacetate) (0.190 g, crude) as a yellow solid. The solid was used directly in the next step without further purification. (LC/MS) m/z (M+H)⁺=386.2.

### Preparation F2A

### 1-(1-Methyl-7-(4-(2-(methylamino)ethyl)piperazin-1-yl)-1H-indazol-3-yl)dihydropyrimidine-2,4(1H,3H)-dione dihydrochloride

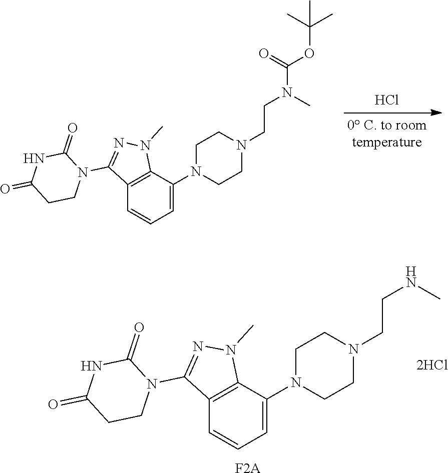

### Step 1. Preparation of 1-(1-methyl-7-(4-(2-(methylamino)ethyl)piperazin-1-yl)-1H-indazol-3-yl)dihydropyrimidine-2,4(1H,3H)-dione dihydrochloride

To a mixture of tert-butyl(2-(4-(3-(2,4-dioxotetrahydropyrimidin-1(2H)-yl)-1-methyl-1H-indazol-7-yl)piperazin-1-yl)ethyl)(methyl)carbamate (0.100 g, 0.206 mmol) in ACN (1 mL) was added concentrated 12 M HCl (22.5 mg, 51.5 μL, 0.618 mmol). The resulting slurry was stirred at room temperature for 2 min then water (0.1 mL) was added. The mixture was stirred at room temperature for 2 h and 30 min before another portion of concentrated 12M HCl (52.6 mg, 120.0 μL, 1.44 mmol) and solvent mixture ACN: H₂O (1 mL:0.35 mL) were added. The reaction mixture was stirred at room temperature for 2 h and 20 min then was diluted with ACN (2 mL). The suspension was stirred then a bi-phasic solution of (9:1, ACN: H₂O)(3 mL: 0.35 mL) was added. The mixture was stirred overnight then additional ACN (1 mL) then the mixture of ACN: H₂O (4 mL:0.35 mL) were added. The suspension was filtered, rinsed by ACN (3×1 mL), dried by vacuum under nitrogen gas to get 1-(1-methyl-7-(4-(2-(methylamino)ethyl)piperazin-1-yl)-1H-indazol-3-yl)dihydropyrimidine-2,4(1H,3H)-dione dihydrochloride (60.0 mg, 63.6% yield) as a solid. (LC/MS) m/z (M+H)⁺=386.3. ¹H NMR (400 MHz, (CD₃)₂SO) δ 11.14 (s, 1H), 10.56 (s, 1H), 9.17 (s, 2H), 7.39 (d, 1H), 7.13-7.02 (m, 2H), 4.26 (s, 3H), 3.88 (t, 2H), 3.77-3.73 (m, 2H), 3.65-3.38 (m, 10H), 2.76 (t, 2H), 2.68-2.61 (m, 3H).

### Preparation F3

### 3-(3-Methyl-4-((4-((methylamino)methyl)piperidin-1-yl)methyl)-2-oxo-2,3-dihydro-1H-benzo[d]imidazol-1-yl)piperidine-2,6-dione bis(2,2,2-trifluoroacetate)

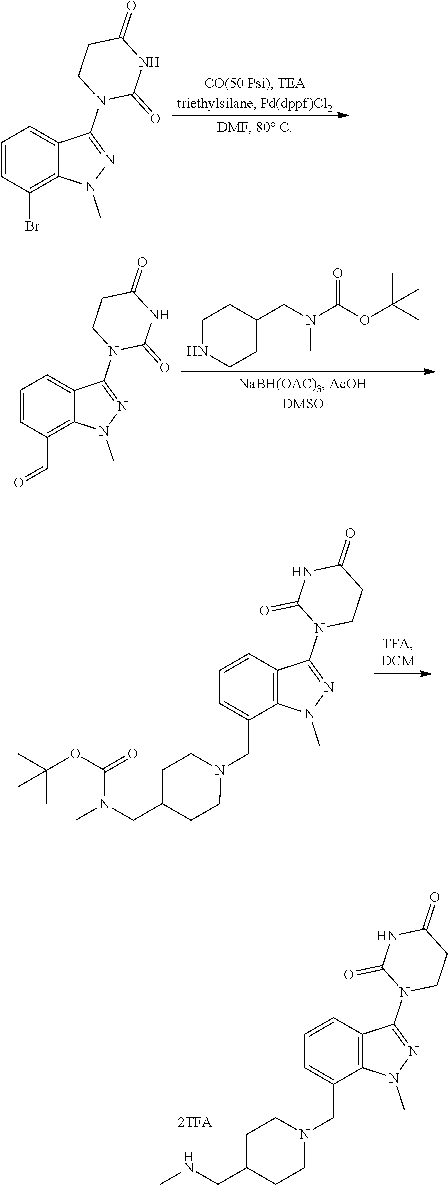

### Step 1. Preparation of 3-(2,4-dioxotetrahydropyrimidin-1(2H)-yl)-1-methyl-1H-indazole-7-carbaldehyde

To a solution of 1-(7-bromo-1-methyl-1H-indazol-3-yl)dihydropyrimidine-2,4(1H,3H)-dione (CAS: 2810876-98-1; 2.40 g, 7.43 mmol) in DMF (30 mL) was added TEA (2.26 g, 22.3 mmol), triethylsilane (2.59 g, 22.3 mmol) and Pd(dppf)Cl₂ (0.607 g, 0.743 mmol). The reaction mixture was degassed with argon gas (3×) then stirred under CO at 50 psi and 80 °C for 36 h. To the mixture was added water (60 mL) then extracted with EtOAc (60 mL×3). The combined organic layer was washed with brine (40 mL×2) then concentrated in vacuo. To the black oil was added DCM (10 mL), which caused precipitate to form. The mixture was filtered then the filter cake was collected to provide 3-(2,4-dioxotetrahydropyrimidin-1(2H)-yl)-1-methyl-1H-indazole-7-carbaldehyde (660 mg) as a brown solid. The aqueous phase was filtered and then the filter cake was collected. The filter cake was triturated with MeOH (3 mL) to give 3-(2,4-dioxotetrahydropyrimidin-1(2H)-yl)-1-methyl-1H-indazole-7-carbaldehyde (380 mg). The two batches of target product were combined to give 3-(2,4-dioxotetrahydropyrimidin-1(2H)-yl)-1-methyl-1H-indazole-7-carbaldehyde (1.04 g, 51.4% yield) as a brown solid. (LC/MS) m/z (M+H)⁺=273.0. ¹H NMR (400 MHz, (CD₃)₂SO) δ 10.62 (s, 1H), 10.27 (s, 1H), 8.08-7.99 (m, 2H), 7.37-7.30 (m, 1H), 4.28 (s, 3H), 3.93 (s, 2H), 2.77 (s, 2H).

### Step 2. Preparation of tert-butyl ((1-((3-(2,4-dioxotetrahydropyrimidin-1(2H)-yl)-1-methyl-1H-indazol-7-yl)methyl)piperidin-4-yl)methyl)(methyl)carbamate

To a solution of 3-(2,4-dioxotetrahydropyrimidin-1(2H)-yl)-1-methyl-1H-indazole-7-carbaldehyde (20.0 g, 73.5 mmol) and tert-butyl methyl(piperidin-4-ylmethyl)carbamate (CAS: 138022-04-5; 16.8 g, 73.5 mmol) in DMSO (230 mL) was added AcOH (4.41 g, 73.5 mmol) then NaBH(OAc)₃ (2.90 g, 137 mmol). The reaction mixture was stirred for 2 h then poured into water (200 mL) and extracted with (1:10, MeOH: DCM)(150 mL×3). The combined organic phase was washed with brine (200 mL×3) then dried over Na₂SO₄, filtered, and concentrated in vacuo to give the tert-butyl ((1-((3-(2,4-dioxotetrahydropyrimidin-1(2H)-yl)-1-methyl-1H-indazol-7-yl)methyl)piperidin-4-yl)methyl)(methyl)carbamate (35.4 g, crude) as a brown solid. The solid was used directly for the next step without further purification.

### Step 3. Preparation of 3-(3-methyl-4-((4-((methylamino)methyl)piperidin-1-yl)methyl)-2-oxo-2,3-dihydro-1H-benzo[d]imidazol-1-yl)piperidine-2,6-dione bis(2,2,2-trifluoroacetate)

At 0 °C, to a solution of tert-butyl ((1-((3-(2,4-dioxotetrahydropyrimidin-1(2H)-yl)-1-methyl-1H-indazol-7-yl)methyl)piperidin-4-yl)methyl)(methyl)carbamate (35.4 g, 73.0 mmol) in DCM (350 mL) was added TFA (70 mL). The reaction mixture was stirred at room temperature for 1.5 h then concentrated in vacuo. The residue was diluted with water (250 mL) then extracted with (1:10, MeOH: DCM) (50 mL×3). The aqueous phase was lyophilized to provide 3-(3-methyl-4-((4-((methylamino)methyl)piperidin-1-yl)methyl)-2-oxo-2,3-dihydro-1H-benzo[d]imidazol-1-yl)piperidine-2,6-dione bis(2,2,2-trifluoroacetate) (37.5 g, crude) as a white solid. The solid was used directly in the next step without further purification. (LC/MS) m/z (M+H)⁺=385.3. ¹H NMR (400 MHz, (CD₃)₂SO) δ 10.59 (s, 1H), 9.84-9.80 (m, 1H), 8.78 (s, 2H), 7.78 (d, 1H), 7.60-7.53 (m, 1H), 7.21 (t, 1H), 4.77-4.73 (m, 2H), 4.24 (s, 3H), 3.89 (t, 2H), 3.55-3.51 (m, 2H), 3.27-3.02 (m, 2H), 2.88-2.71 (m, 4H), 2.63-2.52 (m, 3H), 1.95 (d, 3H), 1.48-1.40 (m, 2H).

### Preparation F4

### 1-(1-Methyl-7-(4-(2-(methylamino)ethyl)piperidin-1-yl)-1H-indazol-3-yl)dihydropyrimidine-2,4(1H,3H)-dione trifluoroacetate

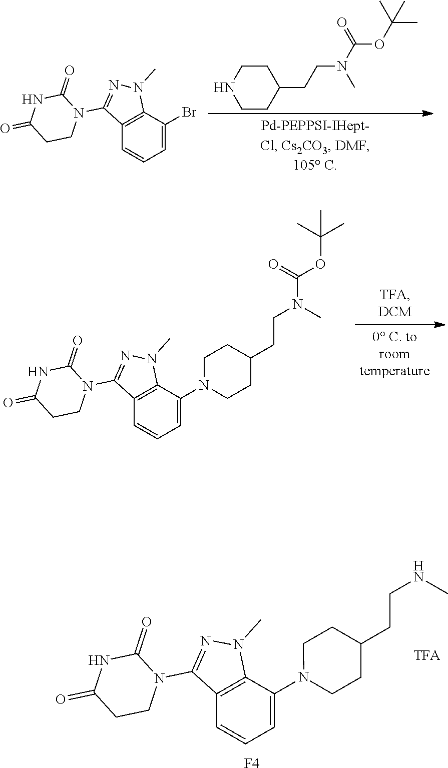

### Step 1. Preparation oftert-butyl(2-(1-(3-(2,4-dioxotetrahydropyrimidin-1(2H)-yl)-1-methyl-1H-indazol-7-yl)piperidin-4-yl)ethyl)(methyl)carbamate

To a solution of 1-(7-bromo-1-methyl-1H-indazol-3-yl)dihydropyrimidine-2,4(1H,3H)-dione (CAS: 3024268-56-9; 2.40 g, 7.43 mmol) and tert-butyl methyl(2-(piperidin-4-yl)ethyl)carbamate (CAS: 171049-32-4; 1.98 mg, 8.17 mmol) in DMF (24 mL) was added Cs₂CO₃ (7.26 g, 22.3 mmol) and Pd-PEPPSI-lHept-CI (CAS: 1814936-54-3; 722 mg, 0.743 mmol) at 105 °C under nitrogen gas. The reaction mixture was stirred at 105 °C for 12 h then diluted with H₂O (150 mL) and extracted with EtOAc (100 mL×3). The organic phase was washed with brine (100 mL×2), dried with Na₂SO₄, then concentrated in vacuo to give a brown gum. The gum was purified by column chromatography (silica gel; 0-80% EtOAc:hexane) to provide tert-butyl(2-(1-(3-(2,4-dioxotetrahydropyrimidin-1(2H)-yl)-1-methyl-1H-indazol-7-yl)piperidin-4-yl)ethyl)(methyl)carbamate (2.10 g, 58.3% yield) as a yellow gum. (LC/MS) m/z (M+H)⁺=485.7. ¹H NMR (400 MHz, (CD₃)₂SO) 6 10.54 (s, 1H), 7.35-7.11 (m, 1H), 7.04-6.96 (m, 2H), 4.23 (s, 3H), 3.88 (t, 2H), 3.31-3.21 (m, 4H), 2.75 (d, 5H), 1.85 (d, 2H), 1.53-1.35 (m, 16H).

### Step 2. Preparation of 1-(1-methyl-7-(4-(2-(methylamino)ethyl)piperidin-1-yl)-1H-indazol-3-yl)dihydropyrimidine-2,4(1H,3H)-dione trifluoroacetate

At 0 °C, to a solution of tert-butyl(2-(1-(3-(2,4-dioxotetrahydropyrimidin-1(2H)-yl)-1-methyl-1H-indazol-7-yl)piperidin-4-yl)ethyl)(methyl)carbamate (2.10 g, 4.33 mmol) in DCM (6 mL) was added TFA (12 mL). The reaction mixture was stirred at room temperature for 1.5 h then concentrated in vacuo. The residue was diluted with H₂O (50 mL) then extracted with DCM (30 mL×3). The organic layers were discarded, and the aqueous phase was lyophilized to give a yellow oil. The oil was triturated with MTBE (10 mL) to give an off-white solid. The solid was lyophilized to give a yellow gum. The gum was triturated with MTBE (6 mL) to give 1-(1-methyl-7-(4-(2-(methylamino)ethyl)piperidin-1-yl)-1H-indazol-3-yl)dihydropyrimidine-2,4(1H,3H)-dione trifluoroacetate (1.10 g, crude) as an off-white solid. The solid was used directly in the next step without further purification. (LC/MS) m/z (M+H)⁺=385.4. ¹H NMR (400 MHz, (CD₃)₂SO) δ 10.46 (s, 1H), 8.50-8.43 (m, 2H), 7.25-7.16 (m, 1H), 6.97-6.88 (m, 2H), 4.14 (s, 3H), 3.79 (t, 2H), 3.16 (d, 2H), 2.94-2.83 (m, 3H), 2.67 (t, 2H), 2.58 (t, 1H), 2.53-2.43 (m, 3H), 1.73 (d, 2H), 1.65-1.30 (m, 5H).

### Preparation F5

### 3-(2,4-Dioxotetrahydropyrimidin-1(2H)-yl)-5-fluoro-1-methyl-1H-indazole-6-carbaldehyde

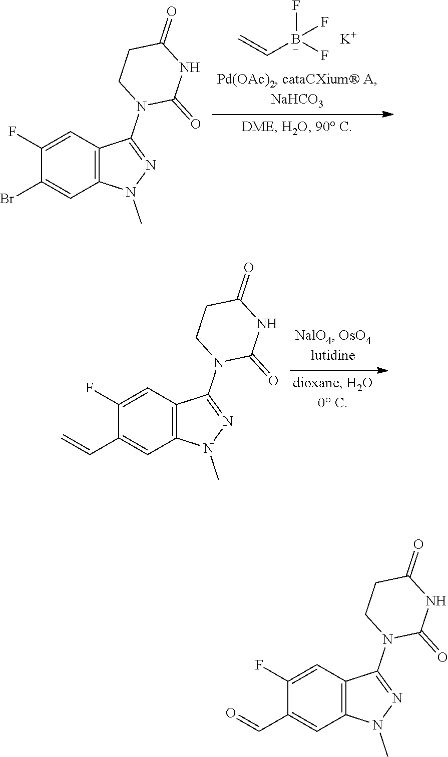

### Step 1. Preparation of 1-(5-fluoro-1-methyl-6-vinyl-1H-indazol-3-yl)dihydropyrimidine-2,4(1H,3H)-dione

A mixture of 1-(6-bromo-5-fluoro-1-methyl-1H-indazol-3-yl)dihydropyrimidine-2,4(1H,3H)-dione (0.600 g, 1.76 mmol), potassium vinyltrifluoroborate (CAS: 13682-77-4; 471 mg, 3.52 mmol), and NaHCO₃ (443 mg, 5.28 mmol) was dissolved in DME (8 mL) and water (2 mL). The reaction mixture was degassed with N₂ gas (3×) at room temperature then Pd(OAc)₂ (39.5 mg, 0.176 mmol) and cataCXium© A (126 mg, 0.352 mmol) were added to the solution. The reaction mixture was degassed with N₂ gas (3×). The resulting reaction mixture was stirred at 90 °C for 16 h then filtered through Celite and washed with DCM and EtOAc (50 mL×2). The combined organic layers were concentrated in vacuo to afford a crude yellow gum. The Celite was further washed with MeOH (50 mL×2). The combined MeOH washings were concentrated in vacuo and combined with the previous crude material to provide a yellow gum. A gum was suspended in a mixture of (1:1:1) (EtOAc: DCM: MeOH) (5 mL) then stirred for 5 min. The suspension was filtered. The filter cake was washed with DCM (3×5 mL) then the filter cake was collected to provide 1-(5-fluoro-1-methyl-6-vinyl-1H-indazol-3-yl)dihydropyrimidine-2,4(1H,3H)-dione (0.200 g, 39.4% yield) as a yellow solid. (LC/MS) m/z (M+H)⁺=288.9. ¹H NMR (400 MHz, (CD₃)₂SO) δ 10.55 (s, 1H), 7.92 (d, 1H), 7.42 (d, 1H), 6.93 (dd, 1H), 6.10 (d, 1H), 5.51 (d, 1H), 4.04-3.97 (m, 3H), 3.91 (t, 2H), 2.76 (t, 2H).

### Step 2. Preparation of 3-(2,4-dioxotetrahydropyrimidin-1(2H)-yl)-5-fluoro-1-methyl-1H-indazole-6-carbaldehyde

To a mixture of 1-(5-fluoro-1-methyl-6-vinyl-1H-indazol-3-yl)dihydropyrimidine-2,4(1H,3H)-dione (207 mg, 0.718 mmol) and lutidine (154 mg, 1.44 mmol) in a solution of dioxane (4 mL) and H₂O (2 mL) at 0 °C were added 0.0 (54.8 mg, 0.215 mmol) and NalO₄ (614 mg, 2.87 mmol). The reaction mixture was stirred at 0 °C for 2 h. The mixture was cooled with ice then treated with saturated aqueous Na₂SO₃ solution (10 mL) then stirred at room temperature for −30 min. To the reaction mixture were added H₂O and saturated aqueous NH₄Cl solution (10 mL). The mixture was extracted with EtOAc (10 mL×5). The combined organic layers were washed with saturated aqueous NaCl solution, dried over anhydrous Na₂SO₄, filtered, and concentrated in vacuo to afford 3-(2,4-dioxotetrahydropyrimidin-1(2H)-yl)-5-fluoro-1-methyl-1H-indazole-6-carbaldehyde (262 mg, crude) as a yellow solid. The solid was used directly in the next step without further purification. (LC/MS) m/z (M+H)⁺=290.9. ¹H NMR (400 MHz, (CD₃)₂SO) δ 10.63 (s, 1H), 10.29 (s, 1H), 8.23 (d, 1H), 7.61 (d, 1H), 4.10 (s, 3H), 3.94 (t, 2H), 2.77 (t, 2H). Preparation F6 rac-(R)-3-(1-Methyl-6-(4-(2-(methylamino)ethyl)piperazin-1-yl)-IH-indazol-3-yl)piperidine-2,6-dione bis(2,2,2-trifluoroacetate)

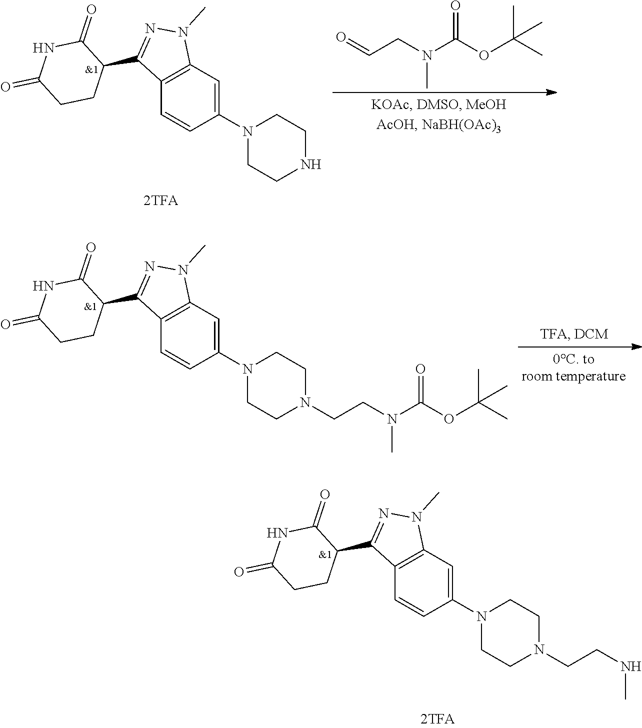 [SMILES: Cn1nc([C@H]2CCC(=O)NC2=O)c2ccc(N3CCNCC3)cc21 ; CN(CC=O)C(=O)OC(C)(C)C ; CN(CCN1CCN(c2ccc3c([C@H]4CCC(=O)NC4=O)nn(C)c3c2)CC1)C(=O)OC(C)(C)C ; CNCCN1CCN(c2ccc3c([C@H]4CCC(=O)NC4=O)nn(C)c3c2)CC1]

### Step 1. Preparation of rac-tert-butyl (R)-(2-(4-(3-(2,6-dioxopiperidin-3-yl)-1-methyl-1H-indazol-6-yl)piperazin-1-yl)ethyl)(methyl)carbamate

To a solution of rac-(R)-3-(1-methyl-6-(piperazin-1-yl)-1H-indazol-3-yl)piperidine-2,6-dione bis(2,2,2-trifluoroacetate) (CAS: 306314-45-6; prepared through the literature synthesis conducted in Preparation of trisubstituted pyrimidine compounds as inhibitors and degraders of PIP4K protein; Inventors: Good, Andrew; Brown, Kristin; Goodman, Krista B.; Ye, Guosen; O'Shea, Morgan; WO2024233846 A1; 0.14 g, 0.25 mmol) and tert-butyl methyl(2-oxoethyl)carbamate (CAS: 123387-72-4; 46 mg, 0.26 mmol) in DMSO (1.50 mL) and MeOH (0.15 mL) was added AcOK (87 mg, 0.88 mmol), AcOH (0.030 g, 0.50 mmol) then NaBH(OAc)₃ (0.11 g, 0.50 mmol). The reaction mixture was degassed with nitrogen gas for 2 min then stirred at room temperature for 4 h then poured into water and extracted with DCM (10 mL×3). The combined organic phase was washed with brine (10 mL×2), dried with Na₂SO₄ and concentrated in vacuo to provide rac-tert-butyl (R)-(2-(4-(3-(2,6-dioxopiperidin-3-yl)-1-methyl-IH-indazol-6-yl)piperazin-1-yl)ethyl)(methyl)carbamate (0.15 g, crude) as a yellow gum. The gum was used directly in the next step without further purification. (LC/MS) m/z (M+H)⁺=485.3.

### Step 2. Preparation of rac-(R)-3-(1-methyl-6-(4-(2-(methylamino)ethyl)piperazin-1-yl)-1H-indazol-3-yl)piperidine-2,6-dione bis(2,2,2-trifluoroacetate)

At 0 °C, to a solution of rac-tert-butyl (R)-(2-(4-(3-(2,6-dioxopiperidin-3-yl)-1-methyl-1H-indazol-6-yl)piperazin-1-yl)ethyl)(methyl)carbamate (0.15 g, 0.32 mmol) in DCM (2.0 mL) was added TFA (0.5 mL) then stirred at room temperature for 1.5 h. The mixture was concentrated in vacuo then diluted with H₂O (10 mL) and extracted with DCM (10 mL×3). The combined organic phase was discarded. The aqueous phase was lyophilized overnight to give rac-(R)-3-(1-methyl-6-(4-(2-(methylamino)ethyl)piperazin-1-yl)-1H-indazol-3-yl)piperidine-2,6-dione bis(2,2,2-trifluoroacetate) (0.21 g, crude) as a black solid. The solid was used directly in the next step without further purification. (LC/MS) m/z (M+H)⁺=385.2. ¹H NMR (400 MHz, (CD₃)₂SO) δ 10.87 (s, 1H), 8.91-8.87 (m, 2H), 7.61-7.54 (m, 1H), 7.02-6.96 (m, 2H), 4.33-4.24 (m, 1H), 3.93 (s, 3H), 3.56-3.34 (m, 6H), 2.70-2.56 (m, 5H), 2.57-2.51 (m, 6H), 2.42-2.25 (m, 1H), 2.20-2.11 (m, 1H).

### Preparation F7

### rac-(R)—N-(2,6-Dioxopiperidin-3-yl)-6-(6-(piperidin-4-yloxy)hex-1-yn-1-yl)picolinamide trifluoroacetate

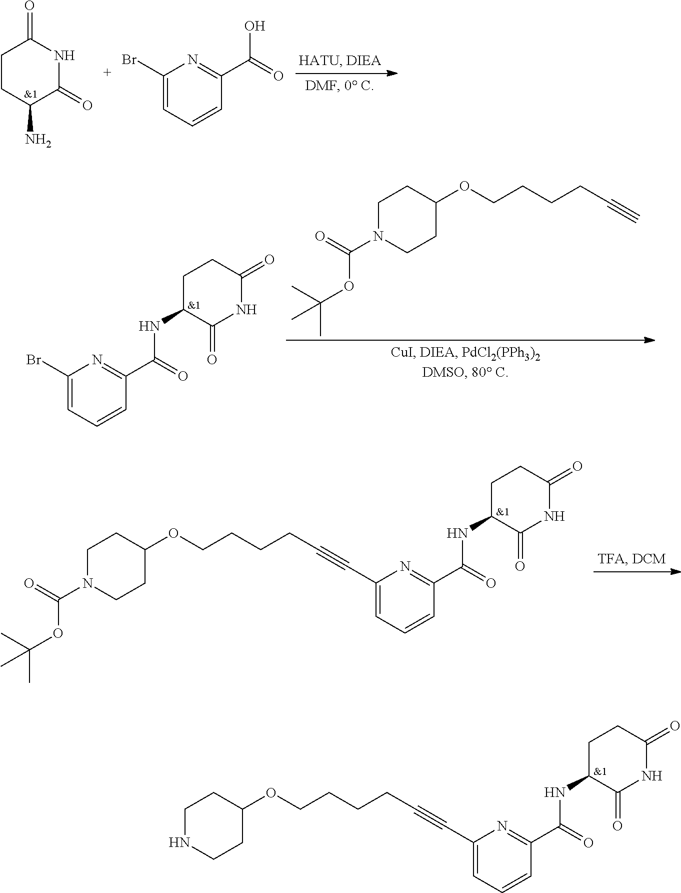

### Step 1. Preparation of rac-(R)-6-bromo-N-(2,6-dioxopiperidin-3-yl)picolinamide

To a solution of rac-(R)-3-aminopiperidine-2,6-dione hydrochloride (CAS: 24666-56-6; 0.500 g, 3.04 mmol) and 6-bromopicolinic acid (CAS: 21190-87-4; 0.614 g, 3.04 mmol) in DMF (2.0 mL) was added HATU (1.21 g, 3.19 mmol). The mixture was stirred at 0 °C for 30 min then warmed to room temperature before DIEA (1.57 g, 12.2 mmol) was added dropwise. The reaction mixture was stirred at room temperature for 16 h then poured into ice water (8 mL). The suspension was extracted with EtOAc (5 mL×3). The combined organic layer was washed with Na₂CO₃ (5 mL×2) and brine (10 mL) then dried with Na₂SO₄. The mixture was concentrated in vacuo to provide rac-(R)-6-bromo-N-(2,6-dioxopiperidin-3-yl)picolinamide (0.300 g, crude) as a white solid. The solid was used directly into the next step without further purification. (LC/MS) m/z (M+H)⁺=312.0. ¹H NMR (400 MHz, (CD₃)₂SO) δ 10.90 (s, 1H), 9.01 (d, 1H), 8.11-8.04 (m, 1H), 7.97 (t, 1H), 7.93-7.86 (m, 1H), 4.86-4.75 (m, 1H), 2.91-2.71 (m, 1H), 2.57-2.51 (m, 1H), 2.32-2.16 (m, 1H), 2.03-1.92 (m, 1H).

### Step 2. Preparation of rac-tert-butyl (R)-4-((6-(6-((2,6-dioxopiperidin-3-yl)carbamoyl)pyridin-2-yl)hex-5-yn-1-yl)oxy)piperidine-1-carboxylate

To the mixture of rac-(R)-6-bromo-N-(2,6-dioxopiperidin-3-yl)picolinamide (0.130 g, 0.417 mmol) and tert-butyl 4-(hex-5-yn-1-yloxy)piperidine-1-carboxylate (CAS: 3007651-52-4; literature reference: Preparation of pyridopyrimidine compounds as KRAS proteolysis targeting chimeras; Inventors: Ji, Nan; Yin, Ning; Qiu, Hui; Lan, Ruoxi; World Intellectual Property Organization, WO2024263586 A1; 0.136 g, 0.482 mmol) in DMSO (3 mL) was added DIEA (0.166 g, 1.28 mmol), CuI (12.2 mg, 0.0642 mmol) and PdCl₂(PPh₃)₂(45.1 mg, 0.0642 mmol). The mixture was degassed with nitrogen gas for 3 min then stirred at 80 °C for 3 h. The reaction was quenched with H₂O (10 mL) and extracted with EtOAc (3×50 mL). The combined organic layers were dried over Na₂SO₄, filtered and concentrated in vacuo. The yellow oil was purified by column chromatography (silica gel; 0-100% EtOAc: petroleum ether) to give rac-tert-butyl (R)-4-((6-(6-((2,6-dioxopiperidin-3-yl)carbamoyl)pyridin-2-yl)hex-5-yn-1-yl)oxy)piperidine-1-carboxylate (0.120 g, 18.2% yield) as a yellow oil. (LC/MS) m/z (M+H)⁺=513.3.

### Step 3. Preparation of rac-(R)—N-(2,6-dioxopiperidin-3-yl)-6-(6-(piperidin-4-yloxy)hex-1-yn-1-yl)picolinamide trifluoroacetate

To the mixture of rac-tert-butyl (R)-4-((6-(6-((2,6-dioxopiperidin-3-yl)carbamoyl)pyridin-2-yl)hex-5-yn-1-yl)oxy)piperidine-1-carboxylate (0.15 g, 0.29 mmol) in DCM (5 mL) was added TFA (1 mL). The suspension was stirred at room temperature for 30 min then concentrated in vacuo and lyophilized to give rac-(R)—N-(2,6-dioxopiperidin-3-yl)-6-(6-(piperidin-4-yloxy)hex-1-yn-1-yl)picolinamide trifluoroacetate (0.060 g, crude) as a black solid. The solid was used directly in next step without further purification. ¹H NMR (400 MHz, (CD₃)₂SO) δ 10.88 (s, 1H), 8.96 (d, 1H), 8.53-8.28 (m, 2H), 8.04-7.94 (m, 2H), 7.72-7.44 (m, 1H), 4.86-4.75 (m, 1H), 3.59-3.39 (m, 3H), 3.23-3.06 (m, 2H), 3.02-2.90 (m, 2H), 2.87-2.74 (m, 1H), 2.56-2.51 (m, 3H), 2.31-2.16 (m, 1H), 2.03-1.86 (m, 3H), 1.72-1.57 (m, 6H).

### Preparation F8

### 1-(5-Bromo-6-fluoropyridin-3-yl)pyrrolidin-2-one (F8)

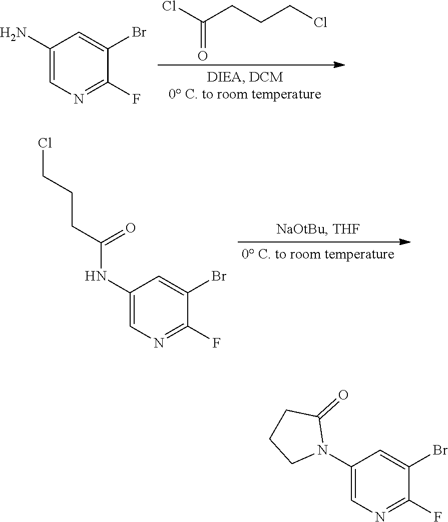

### Step 1. Preparation of N-(5-bromo-6-fluoropyridin-3-yl)-4-chlorobutanamide

At 0 °C, to a solution of 5-bromo-6-fluoropyridin-3-amine (CAS: 209328-99-4; 4.50 g, 23.6 mmol) in DCM (45 mL) were added 4-chlorobutanoyl chloride (CAS: 4635-59-0; 4.98 g, 35.3 mmol) and DIEA (10.7 g, 82.5 mmol). The reaction mixture was stirred at room temperature for 2 h, then poured into H₂O (60 mL) and extracted with DCM (3×30 mL). The combined organic layers were washed with H₂O, dried over Na₂SO₄, and concentrated in vacuo. The residue was purified by column chromatography (silica gel; 0-50% EtOAc: n-hexane) to provide N-(5-bromo-6-fluoropyridin-3-yl)-4-chlorobutanamide (5.02 g, 72.1% yield) as a yellow solid. (LC/MS) m/z (M+H)⁺=296.8.

### Step 2. Preparation of 1-(5-bromo-6-fluoropyridin-3-yl)pyrrolidin-2-one

At 0 °C, to a solution of N-(5-bromo-6-fluoropyridin-3-yl)-4-chlorobutanamide (5.02 g, 17.0 mmol) in THF (55 mL) was added NaOtBu (2.45 g, 25.5 mmol). The reaction mixture was stirred at room temperature for 20 min then poured into H₂O (100 mL) and extracted with EtOAc (3×60 mL). The combined organic layers were washed with H₂O (1×), dried over Na₂SO₄, and concentrated in vacuo. The residue was purified by column chromatography (silica gel; 0-80% EtOAc: n-hexane) to provide 1-(5-bromo-6-fluoropyridin-3-yl)pyrrolidin-2-one (2.45 g, 55.7% yield) as a yellow solid. (LC/MS) m/z (M+H)⁺=258.9.

### Preparation F9

### 1-(6-Fluoro-1-methyl-7-(4-(2-(methylamino)ethyl)piperazin-1-yl)-1H-indazol-3-yl)dihydropyrimidine-2,4(1H,3H)-dione trifluoroacetate

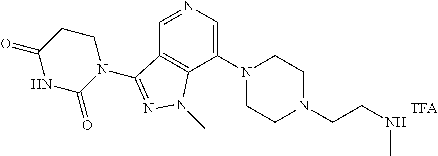 [SMILES: CNCCN1CCN(c2cncc3c(N4CCC(=O)NC4=O)nn(C)c23)CC1]

Synthesized according to Preparation F4. LC/MS m/z [M+H]⁺=404.3. ¹H NMR (400 MHz, (CD₃)₂SO) δ 10.59 (s, 1H), 8.71-8.67 (m, 2H), 7.66-7.46 (m, 1H), 7.10-6.96 (m, 1H), 4.26 (s, 3H), 3.88 (t, 2H), 3.62-3.25 (m, 12H), 2.76 (t, 2H), 2.66 (s, 3H).

The Buchwald-Hartwig amination conditions were altered. To a solution of 1-(7-bromo-6-fluoro-1-methyl-1H-indazol-3-yl)dihydropyrimidine-2,4(1H,3H)-dione (0.200 g, 0.527 mmol) and tert-butyl methyl(2-(piperazin-1-yl)ethyl)carbamate (CAS: 1540928-69-5; 192 mg, 0.790 mmol) in tert-amyl alcohol (4 mL) were added NaOtBu (203 mg, 2.11 mmol), 4A molecular sieves (0.200 g), and GPhos Pd G6 TES (99.5 mg, 0.105 mmol). The reaction mixture was stirred at 100 °C under nitrogen for 16 h. After cooling, the reaction mixture was filtered and concentrated in vacuo. The reside was purified by column chromatography (silica gel; 0-10% MeOH: DCM) to afford tert-butyl (2-(4-(3-(2,4-dioxotetrahydropyrimidin-1(2H)-yl)-6-fluoro-1-methyl-1H-indazol-7-yl)piperazin-1-yl)ethyl)(methyl)carbamate (61.0 mg, 23.0% yield).

### Preparation F10

### 1-(5-Fluoro-1-methyl-7-(4-(2-(methylamino)ethyl)piperazin-1-yl)-1H-indazol-3-yl)dihydropyrimidine-2,4(1H,3H)-dione bis(2,2,2-trifluoroacetate)

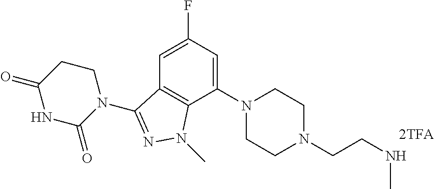 [SMILES: CNCCN1CCN(c2cc(F)cc3c(N4CCC(=O)NC4=O)nn(C)c23)CC1]

Synthesized according to Preparation F4. LC/MS m/z [M+H]⁺=404.1.

The Buchwald-Hartwig amination conditions were altered. To a solution of 1-(7-bromo-5-fluoro-1-methyl-1H-indazol-3-yl)dihydropyrimidine-2,4(1H,3H)-dione (CAS: 2983060-17-7; 0.360 g, 1.06 mmol) and tert-butyl methyl(2-(piperazin-1-yl)ethyl)carbamate (CAS: 1540928-69-5; 308 mg, 1.27 mmol) in tert-amyl alcohol (6 mL) were added NaOtBu (304 mg, 3.17 mmol), 4-methylbenzenesulfonamide (300 mg, 0 mmol), and RuPhos Pd G4 (89.7 mg, 0.106 mmol). The reaction mixture was stirred at 60 °C under nitrogen for 16 h. After completion, the mixture was 5 filtered. The filter cake was washed with EtOAc. The filtrate was partitioned with water (30 mL). The aqueous layer was extracted with EtOAc (3×20 mL). The combined organic layers were dried over Na₂SO₄ then purified by column chromatography (silica gel; 0-20% MeOH: EtOAc) to provide tert-butyl(2-(4-(3-(2,4-dioxotetrahydropyrimidin-1(2H)-yl)-5-fluoro-1-methyl-1H-indazol-7-yl)piperazin-1-yl)ethyl)(methyl)carbamate (125 mg, 23.5% yield) as a light brown glass.

### Preparation F11

### 1-(7-Chloro-1-methyl-1H-pyrazolo[3,4-c]pyridin-3-yl)-3-(4-methoxybenzyl)dihydropyrimidine-2,4(1H,3H)-dione

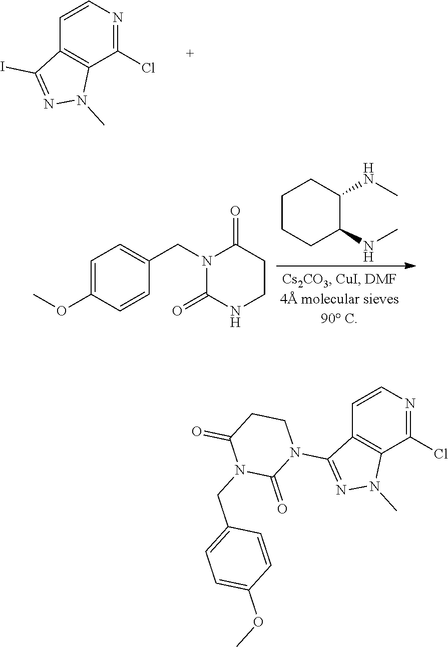

### Step 1. Preparation of 1-(7-chloro-1-methyl-1H-pyrazolo[3,4-c]pyridin-3-yl)-3-(4-methoxybenzyl)dihydropyrimidine-2,4(1H,3H)-dione

The same reaction was conducted in two batches then combined for purification. To a solution of 7-chloro-3-iodo-1-methyl-1H-pyrazolo[3,4-c]pyridine (known in literature from Barvian, Kevin; et al. Preparation of pyrrolo-pyrimidine compounds as cyclin-dependent kinase 4 degraders. WO2025235363; 3.00 g, 10.2 mmol), (1S,2S)—N¹,N²-dimethylcyclohexane-1,2-diamine (CAS: 2631060-00-7; 291 mg, 2.04 mmol), Cs₂CO₃ (6.66 g, 20.4 mmol), 4A molecular sieves (1.00 g), and CuI (389 mg, 2.04 mmol) in DMF (60 mL) was added 3-(4-methoxybenzyl)dihydropyrimidine-2,4(1H,3H)-dione (2.42 g, 10.3 mmol) to form the first batch. The reaction mixture of the first batch was stirred at 90 °C for 16 h.

The second batch was conducted with 7-chloro-3-iodo-1-methyl-1H-pyrazolo[3,4-c]pyridine (0.500 g, 1.70 mmol). The two batches were combined then quenched with H₂O (300 mL) and filtered. The filter cake was dissolved in a mixture of (1:1, DCM: EtOAc) (2×100 mL), washed with brine (2×50 mL), then concentrated in vacuo. The residue was purified by column chromatography (silica gel; 0-7% EtOAc: DCM) to provide 1-(7-chloro-1-methyl-1H-pyrazolo[3,4-c]pyridin-3-yl)-3-(4-methoxybenzyl)dihydropyrimidine-2,4(1H,3H)-dione (3.10 g, 65.0% yield) as a white solid. ¹H NMR (400 MHz, (CD₃)₂SO) δ 8.01 (d, 1H), 7.63 (d, 1H), 7.28-7.21 (m, 2H), 6.91-6.84 (m, 2H), 4.85 (s, 2H), 4.32 (s, 3H), 3.97 (t, 2H), 3.72 (s, 3H), 2.97 (t, 2H).

### Preparation F12

### 1-(1-Methyl-7-(4-(2-(methylamino)ethyl)piperazin-1-yl)-1H-pyrazolo[3,4-c]pyridin-3-yl)dihydropyrimidine-2,4(1H,3H)-dione From E645

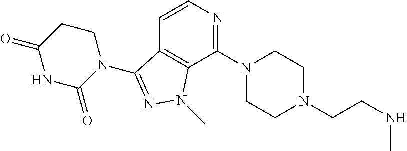 [SMILES: CNCCN1CCN(c2nccc3c(N4CCC(=O)NC4=O)nn(C)c23)CC1]

Synthesized according to Preparation F4 with Preparation F11 but with a PMB deprotection through conditions such as methanesulfonic acid in toluene stirred at 100 °C for 3 h. LC/MS m/z [M+H]⁺=387.2.

The Buchwald-Hartwig amination were altered. To a solution of F11 (0.250 g, 0.625 mmol) and tert-butyl methyl(2-(piperazin-1-yl)ethyl)carbamate (CAS: 1540928-69-5; 183 mg, 0.750 mmol) in DMF (5 mL) were added RuPhos Pd G3 (78.4 mg, 0.0938 mmol) and K₃PO₄ (255 mg, 1.88 mmol) under a nitrogen atmosphere at room temperature. The reaction mixture was then stirred to 100 °C for 12 h. After cooling, the reaction mixture was diluted with H₂O (15 mL) then extracted with EtOAc (3×15 mL). The combined organic layers were washed with brine (3×10 mL), dried over Na₂SO₄, filtered, and concentrated in vacuo. The residue was purified by column chromatography (silica gel; 0-13% MeOH: DCM) to afford tert-butyl(2-(4-(3-(3-(4-methoxybenzyl)-2,4-dioxotetrahydropyrimidin-1(2H)-yl)-1-methyl-1H-pyrazolo[3,4-c]pyridin-7-yl)piperazin-1-yl)ethyl)(methyl)carbamate (80.0 mg, 21.1% yield) as a yellow solid.

### Preparation F13

### rac-(R)-3-(1-Methyl-6-(1-(2-(methylamino)ethyl)piperidin-4-yl)-1H-indazol-3-yl)piperidine-2,6-dione bis(2,2,2-trifluoroacetate)

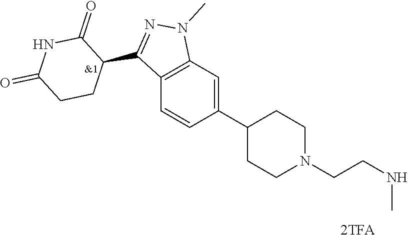 [SMILES: CNCCN1CCC(c2ccc3c([C@H]4CCC(=O)NC4=O)nn(C)c3c2)CC1 ; "2TFA"]

Synthesized according to Preparation F6 with CAS: 3067882-61-2. LC/MS m/z [M+H]⁺=384.3. ¹H NMR (400 MHz, (CD₃)₂SO) δ 10.90 (s, 1H), 10.35-10.30 (m, 1H), 9.10 (s, 2H), 7.68 (d, 1H), 7.42 (s, 1H), 7.05 (d, 1H), 4.35 (dd, 1H), 4.16-4.01 (m, 3H), 3.98-3.89 (m, 3H), 3.72-3.65 (m, 2H), 3.26-3.09 (m, 2H), 3.06-2.93 (m, 1H), 2.73-2.53 (m, 6H), 2.43-2.29 (m, 1H), 2.22-2.00 (m, 5H).

### Preparation F14

### rac-(R)-3-(1-Methyl-7-(4-(2-(methylamino)ethyl)piperazin-1-yl)-1H-indazol-3-yl)piperidine-2,6-dione dihydrochloride

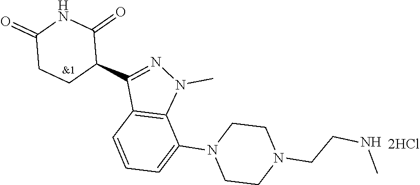 [SMILES: CNCCN1CCN(c2cccc3c([C@H]4CCC(=O)NC4=O)nn(C)c23)CC1]

Synthesized according to Preparation F6 from CAS: 3011028-46-6. LC/MS m/z [M+H]⁺=385.3.

### Preparation F15

### rac-(R)-3-(1-Methyl-6-(4-(2-(methylamino)ethyl)piperidin-1-yl)-1H-indazol-3-yl)piperidine-2,6-dione hydrochloride

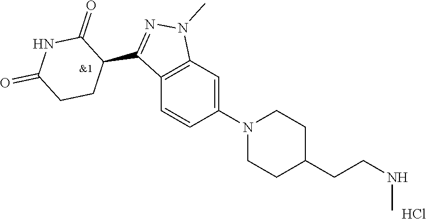 [SMILES: CNCCC1CCN(c2ccc3c([C@H]4CCC(=O)NC4=O)nn(C)c3c2)CC1 ; Cl]

Synthesized according to Preparation F4 from CAS: 2913408-82-7. The deprotection conditions were altered to utilize 4M HCl in dioxane with DCM as a solvent. LC/MS m/z [M+H]⁺=384.3. ¹H NMR (400 MHz, (CD₃)₂SO) δ 10.90 (s, 1H), 8.73 (s, 2H), 7.75-7.71 (m, 1H), 7.32-7.27 (m, 1H), 4.38-4.34 (m, 1H), 3.98 (s, 3H), 3.75-3.66 (m, 2H), 3.57 (s, 5H), 3.02-2.90 (m, 1H), 2.73-2.54 (m, 5H), 2.40-2.33 (m, 1H), 2.23-2.11 (m, 1H), 1.92-1.87 (m, 2H), 1.74-1.62 (m, 4H).

The Buchwald-Hartwig amination conditions were altered. To a reaction mixture of tert-butyl methyl(2-(piperidin-4-yl)ethyl)carbamate (226 mg, 0.931 mmol) and rac-(R)-3-(6-bromo-1-methyl-1H-indazol-3-yl)piperidine-2,6-dione (0.300 g, 0.931 mmol) were added RuPhos (43.5 mg, 93.1 μmol), Pd₂(dba)₃ (42.6 mg, 46.6 μmol) and dioxane (9.31 mL). The mixture was degassed for 10 min before 1 M LiHMDS in THF (799 mg, 4.66 mL, 4.66 mmol) was added. The reaction mixture was stirred at 90 °C for 1 h, then stirred at room temperature for an additional 5 h. The mixture was cooled to 0 °C before AcOH (559 mg, 533 μL, 9.31 mmol) in dioxane (6 mL) was added then filtered through Celite. The filter cake was rinsed with ACN. Three additional portions of AcOH (110 μL) were added. The resulting cloudy solution was stored in the freezer overnight then concentrated in vacuo at temperatures below 40 °C. The residue was purified by column chromatography (silica gel; 0-50% (20% MeOH): DCM) to give rac-tert-butyl (R)-(2-(1-(3-(2,6-dioxopiperidin-3-yl)-1-methyl-1H-indazol-6-yl)piperidin-4-yl)ethyl)(methyl)carbamate (0.224 g, 49.8% yield) as a yellow residue.

### Preparation F16

### 1-(7-((4-(2-(Methylamino)ethyl)piperidin-1-yl)methyl)imidazo[1,2-a]pyridin-3-yl)dihydropyrimidine-2,4(1H,3H)-dione trifluoroacetate

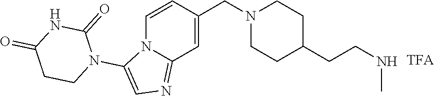 [SMILES: CNCCC1CCN(Cc2ccn3c(N4CCC(=O)NC4=O)cnc3c2)CC1]

Synthesized according to Preparation F3 with CAS: 2925090-59-9. LC/MS m/z [M+H]⁺=385.1. ¹H NMR (400 MHz, (CD₃)₂SO) δ 10.79 (s, 1H), 8.62 (d, 1H), 8.45-8.41 (m, 2H), 7.91 (s, 2H), 7.27 (d, 1H), 4.44 (s, 2H), 3.86-3.78 (m, 2H), 3.48-3.43 (m, 2H), 2.93-2.81 (m, 6H), 2.59-2.50 (m, 3H), 1.90-1.82 (m, 2H), 1.54-1.46 (m, 3H), 1.43-1.32 (m, 2H).

### Preparation F17

### 1-(7-(1-(2-(Methylamino)ethyl)piperidin-4-yl)imidazo[1,2-a]pyridin-3-yl)dihydropyrimidine-2,4(1H,3H)-dione bis(2,2,2-trifluoroacetate)

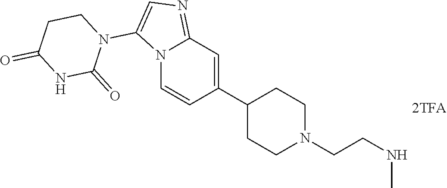 [SMILES: CNCCN1CCC(c2ccn3c(N4CCC(=O)NC4=O)cnc3c2)CC1]

Synthesized according to Preparation F6 with CAS: 2785396-74-7. LC/MS m/z [M+H]⁺=371.3. ¹H NMR (400 MHz, (CD₃)₂SO) δ 10.81 (s, 1H), 9.09-9.05 (m, 1H), 8.69 (d, 1H), 8.03 (s, 1H), 7.74 (s, 1H), 7.34 (d, 1H), 3.84 (t, 3H), 3.66-3.61 (m, 2H), 3.39 (s, 4H), 3.17-3.00 (m, 3H), 2.87-2.82 (m, 2H), 2.65 (s, 3H), 2.53-2.51 (m, 1H), 2.18-1.93 (m, 4H).

### Preparation F18

### rac-(R)-3-(3-Methyl-5-((4-(4-(methylamino)phenyl)piperidin-1-yl)methyl)-2-oxo-2,3-dihydro-1H-benzo[d]imidazol-1-yl)piperidine-2,6-dione bis(2,2,2-trifluoroacetate)

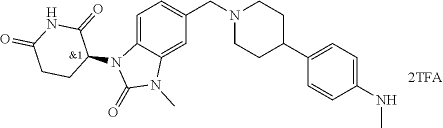 [SMILES: CNc1ccc(C2CCN(Cc3ccc4c(c3)n(C)c(=O)n4[C@H]3CCC(=O)NC3=O)CC2)cc1]

Synthesized according to Preparation F3 from CAS: 2357116-00-6. LC/MS m/z [M+H]⁺=462.3. ¹H NMR (400 MHz, (CD₃)₂SO) δ 11.14 (s, 1H), 9.64-9.54 (m, 1H), 7.38-7.31 (m, 1H), 7.29-7.18 (m, 2H), 7.14-7.07 (m, 2H), 6.97-6.83 (m, 2H), 5.42 (dd, 1H), 4.38-4.31 (m, 2H), 3.51-3.43 (m, 2H), 3.40-3.32 (m, 3H), 3.13-2.85 (m, 3H), 2.81-2.56 (m, 6H), 2.07-1.75 (m, 5H).

### Preparation F19

### 1-(4-Fluoro-1-methyl-7-(4-(2-(methylamino)ethyl)piperazin-1-yl)-1H-indazol-3-yl)dihydropyrimidine-2,4(1H,3H)-dione bis(2,2,2-trifluoroacetate)

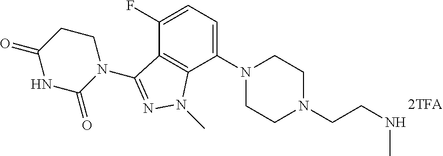 [SMILES: CNCCN1CCN(c2ccc(F)c3c(N4CCC(=O)NC4=O)nn(C)c23)CC1]

Synthesized according to Preparation F10 with CAS: 2983059-93-2. LC/MS m/z [M+H]⁺=404.3.

### Preparation F20

### rac-1-(6-((3R,4S)-3-Fluoro-1-(2-(methylamino)ethyl)piperidin-4-yl)-1-methyl-1H-indazol-3-yl)dihydropyrimidine-2,4(1H,3H)-dione bis(2,2,2-trifluoroacetate)

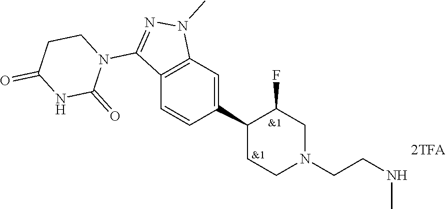 [SMILES: CNCCN1CC[C@@H](c2ccc3c(N4CCC(=O)NC4=O)nn(C)c3c2)[C@@H](F)C1]

Synthesized according to Preparation F6 with CAS: 2654826-60-3. LC/MS m/z [M+H]⁺=403.2.

### Preparation F21

### 1-(1-Methyl-6-((4-(2-(methylamino)ethyl)piperidin-1-yl)methyl)-1H-indazol-3-yl)dihydropyrimidine-2,4(1H,3H)-dione bis(2,2,2-trifluoroacetate)

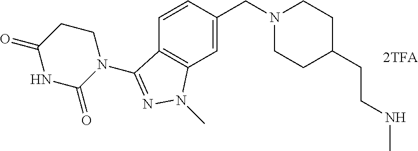 [SMILES: CNCCC1CCN(Cc2ccc3c(N4CCC(=O)NC4=O)nn(C)c3c2)CC1]

Synthesized according to Preparation F3 with CAS: 2925088-72-6. LC/MS m/z [M+H]⁺=399.2.

### Preparation F22

### 1-(6-Bromo-5,7-difluoro-1-methyl-1H-indazol-3-yl)dihydropyrimidine-2,4(1H,3H)-dione

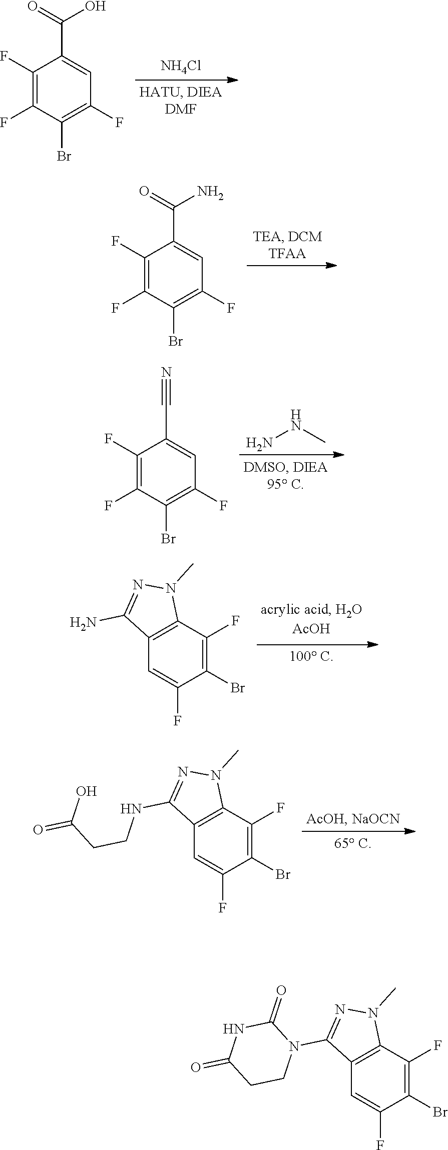

### Step 1. Preparation of 4-bromo-2,3,5-trifluorobenzamide

To a solution of 4-bromo-2,3,5-trifluorobenzoic acid (0.950 g, 3.73 mmol) and NH₄Cl (239 mg, 4.47 mmol) in DMF (10 mL) were added HATU (1.44 g, 3.80 mmol) and DIEA (2.41 g, 18.6 mmol) at room temperature. The reaction mixture was stirred at room temperature for 1 h then poured into H₂O (100 mL) and extracted with EtOAc (3×50 mL). The combined organic layers were washed with saturated aqueous NH₄Cl solution (2×40 mL), dried over Na₂SO₄, and concentrated in vacuo to afford 4-bromo-2,3,5-trifluorobenzamide (0.640 g, 67.6% yield) as a brown solid. LC/MS m/z [M+H]⁺=254.0.

### Step 2. Preparation of 4-bromo-2,3,5-trifluorobenzonitrile

To a solution of 4-bromo-2,3,5-trifluorobenzamide (0.200 g, 0.787 mmol) and TEA (239 mg, 2.36 mmol) in DCM (4 mL) was added TFAA (248 mg, 1.18 mmol) dropwise. The reaction mixture was stirred at room temperature for 2 h. The mixture was then poured into water (10 mL) then the layers were separated. The aqueous layer was extracted with DCM (10 mL). The combined organic layers were washed with brine, dried over Na₂SO₄, and concentrated in vacuo as a black oil. The crude oil was purified by column chromatography (silica gel; 0-10% EtOAc: petroleum ether) to provide 4-bromo-2,3,5-trifluorobenzonitrile (0.120 g, 64.6% yield) as a white solid. ¹H NMR (400 MHz, (CD₃)₂SO) δ 8.16-8.05 (m, 1H).

### Step 3. Preparation of 6-bromo-5,7-difluoro-1-methyl-1H-indazol-3-amine

Under nitrogen gas, to a solution of 4-bromo-2,3,5-trifluorobenzonitrile (3.20 g, 13.6 mmol) in DMSO (26 mL) was added DIEA (5.26 g, 40.7 mmol) followed by methylhydrazine (3.47 g, 30.0 mmol) dropwise at room temperature. The reaction mixture was stirred at 95 °C for 16 h then diluted with water (51 mL) which caused precipitate to form. The suspension was filtered then then filter cake was washed with water (5 mL) and dried under vacuum to afford 6-bromo-5,7-difluoro-1-methyl-1H-indazol-3-amine (2.40 g, crude) as a solid. The solid was used directly in the following steps without further purification. LC/MS m/z [M+H]⁺=264.0. ¹H NMR (400 MHz, (CD₃)₂SO) δ 7.48 (dd, 1H), 5.61 (s, 2H), 3.80-3.75 (m, 3H).

### Step 4. Preparation of 3-((6-bromo-5,7-difluoro-1-methyl-1H-indazol-3-yl)amino)propanoic acid

To a white suspension of 6-bromo-5,7-difluoro-1-methyl-1H-indazol-3-amine (1.00 g, 3.82 mmol) and acrylic acid (1.10 g, 15.3 mmol) in water (20 mL) was added AcOH (992 mg, 16.5 mmol). The reaction mixture was stirred at 100 °C for 48 h before an additional portion of AcOH (115 mg, 1.91 mmol) and acrylic acid (0.550 g, 7.63 mmol) were added. The reaction mixture was stirred at 100 °C for 16 h then another portion of acrylic acid (825 mg, 11.4 mmol) was added. The reaction mixture was stirred at 100 °C for 16 h. The resulting yellow suspension was filtered. The filter cake was collected to afford a yellow solid. The yellow solid was suspended in water (300 mL) then stirred for 1 h, filtered, and lyophilized to provide 3-((6-bromo-5,7-difluoro-1-methyl-1H-indazol-3-yl)amino)propanoic acid (0.800 g, crude) as a yellow solid. The solid was used directly into the next step without further purification. LC/MS m/z [M+H]⁺=335.7.

### Step 5. Preparation of 1-(6-bromo-5,7-difluoro-1-methyl-1H-indazol-3-yl)dihydropyrimidine-2,4(1H,3H)-dione

To a stirred solution of 3-((6-bromo-5,7-difluoro-1-methyl-1H-indazol-3-yl)amino)propanoic acid (2.80 g, 8.38 mmol) in AcOH (42 mL) was added NaOCN (1.09 g, 16.8 mmol) at room temperature. The reaction mixture was stirred at 65 °C for 16 h. The brown solution was cooled to room temperature then 4M HCl (28 mL) was added and stirred at 65 °C for 4 h. The reaction mixture was cooled to room temperature then filtered. The filter cake was collected and set aside.

The filtrate was treated with water (200 mL), which caused yellow precipitate to form. The suspension was filtered and the filter cake was collected.

The filter cakes were combined then lyophilized. to afford 1-(6-bromo-5,7-difluoro-1-methyl-1H-indazol-3-yl)dihydropyrimidine-2,4(1H,3H)-dione (0.700 g, crude) as a brown solid. The solid was used directly into the next step without further purification. LC/MS m/z [M+H]⁺=359.1. ¹H NMR (400 MHz, (CD₃)₂SO) δ 10.62 (s, 1H), 7.51 (dd, 1H), 4.11 (d, 3H), 3.96-3.84 (m, 2H), 2.76 (t, 2H).

### Preparation F23

### 1-(5,7-Difluoro-1-methyl-6-(1-(2-(methylamino)ethyl)piperidin-4-yl)-1H-indazol-3-yl)dihydropyrimidine-2,4(1H,3H)-dione bis(2,2,2-trifluoroacetate)

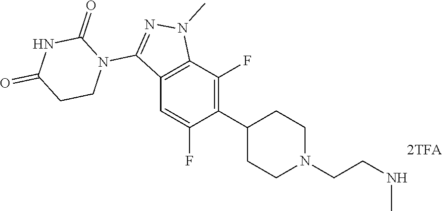 [SMILES: CNCCN1CCC(c2c(F)cc3c(N4CCC(=O)NC4=O)nn(C)c3c2F)CC1]

Made in the manner of Preparation F1 from Preparation F22. LC/MS m/z [M+H]⁺=421.2. ¹H NMR (400 MHz, (CD₃)₂SO) δ 10.62 (s, 1H), 10.16-9.78 (m, 1H), 9.03-8.69 (m, 1H), 7.34 (d, 1H), 4.11 (s, 3H), 3.89 (t, 3H), 3.79-3.46 (m, 6H), 2.75 (t, 2H), 2.70-2.58 (m, 4H), 2.35-2.22 (m, 2H), 2.01-1.97 (m, 2H). The reduction conditions were altered. To a solution of tert-butyl 4-(3-(2,4-dioxotetrahydropyrimidin-1(2H)-yl)-5,7-difluoro-1-methyl-1H-indazol-6-yl)-3,6-dihydropyridine-1(2H)-carboxylate (0.34 g, 0.74 mmol) in THF (6 mL) and DMF (2 mL) were added Pd(OH)₂/C (10 wt %, 0.10 g, 0.074 mmol) and Pd/C (10 wt %, 78 mg, 0.074 mmol). The reaction mixture was stirred under a hydrogen gas at 15 °C and 20 psi for 16 h. The reaction mixture was then filtered through a pad of Celite. The filtrate was concentrated under reduced pressure to afford tert-butyl 4-(3-(2,4-dioxotetrahydropyrimidin-1(2H)-yl)-5,7-difluoro-1-methyl-1H-indazol-6-yl)piperidine-1-carboxylate (340 mg, crude) as a white solid. The solid was used directly into the next step without further purification.

### Preparation F24

### 1-(6-(4-Fluoro-1-(2-(methylamino)ethyl)piperidin-4-yl)-1-methyl-1H-indazol-3-yl)dihydropyrimidine-2,4(1H,3H)-dione bis(2,2,2-trifluoroacetate)

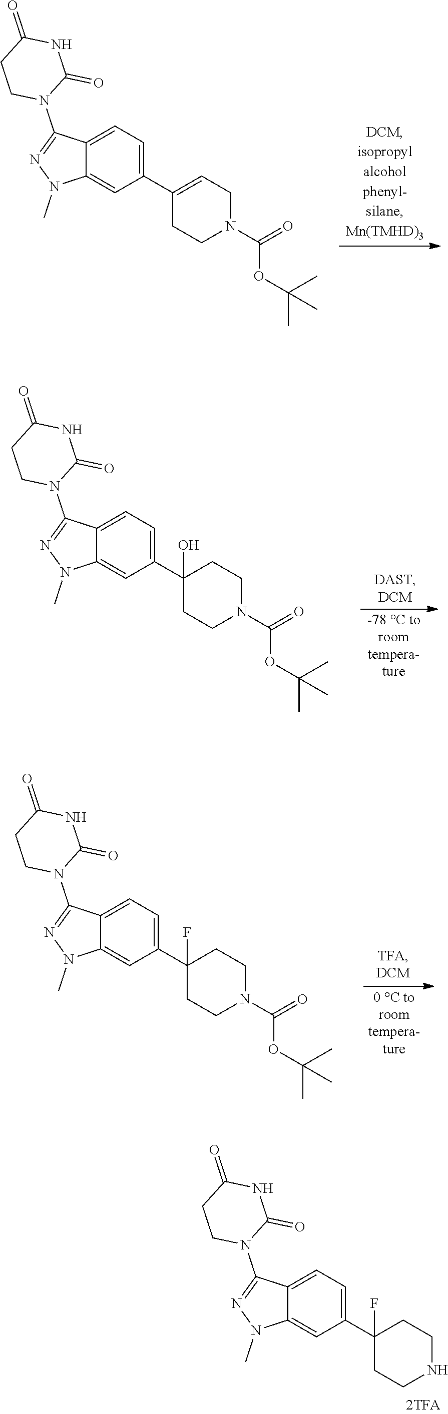 [SMILES: Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3(O)CCN(C(=O)OC(C)(C)C)CC3)cc21 ; Cn1nc(N2CCC(=O)NC2=O)c2ccc(C3(F)CCN(C(=O)OC(C)(C)C)CC3)cc21 ; Cn1nc(N2CCC(=O)NC2=O)c2ccc(C3(F)CCNCC3)cc21]

### Step 1. Preparation of tert-butyl 4-(3-(2,4-dioxotetrahydropyrimidin-1(2H)-yl)-1-methyl-1H-indazol-6-yl)-4-hydroxypiperidine-1-carboxylate

Three batches of the same reaction were conducted then combined for purification. To a solution of tert-butyl 4-(3-(2,4-dioxotetrahydropyrimidin-1(2H)-yl)-1-methyl-1H-indazol-6-yl)-3,6-dihydropyridine-1(2H)-carboxylate (4.60 g, 11.0 mmol) in DCM (50 mL) and isopropyl alcohol (20 mL) were added phenylsilane (1.75 g, 16.2 mmol) and Mn(TMHD)₃ (654 mg, 1.08 mmol) to form the first batch. The reaction mixture of the first batch was stirred at room temperature for 16 h.

The second batch was conducted with tert-butyl 4-(3-(2,4-dioxotetrahydropyrimidin-1(2H)-yl)-1-methyl-1H-indazol-6-yl)-3,6-dihydropyridine-1(2H)-carboxylate (4.60 g, 11.0 mmol) and the third batch was conducted with tert-butyl 4-(3-(2,4-dioxotetrahydropyrimidin-1(2H)-yl)-1-methyl-1H-indazol-6-yl)-3,6-dihydropyridine-1(2H)-carboxylate (0.500 g, 1.18 mmol). The three batches were combined then quenched with water (100 mL) and extracted with DCM (2×50 mL). The combined organic layer was concentrated under reduced pressure. The residue was purified by column chromatography (silica gel; 0-65% EtOAc: DCM) to provide tert-butyl 4-(3-(2,4-dioxotetrahydropyrimidin-1(2H)-yl)-1-methyl-1H-indazol-6-yl)-4-hydroxypiperidine-1-carboxylate (1.32 g, 13.1% yield) as a white solid. LC/MS m/z [M+H]⁺=444.3. ¹H NMR (400 MHz, (CD₃)₂SO) δ 10.55 (s, 1H), 7.65 (s, 1H), 7.58 (d, 1H), 7.31-7.24 (m, 1H), 5.23 (s, 1H), 3.99-3.85 (m, 7H), 3.27-3.10 (m, 2H), 2.75 (t, 2H), 2.01-1.86 (m, 2H), 1.67-1.60 (m, 2H), 1.43 (s, 9H).

### Step 2. Preparation of tert-butyl 4-(3-(2,4-dioxotetrahydropyrimidin-1(2H)-yl)-1-methyl-1H-indazol-6-yl)-4-fluoropiperidine-1-carboxylate

Under a nitrogen gas, a solution of tert-butyl 4-(3-(2,4-dioxotetrahydropyrimidin-1(2H)-yl)-1-methyl-1H-indazol-6-yl)-4-hydroxypiperidine-1-carboxylate (0.100 g, 0.225 mmol) in DCM (3 mL) was cooled to −78 °C. To the cooled reaction mixture, was added DAST (72.7 mg, 0.451 mmol) dropwise. The reaction mixture was warmed to room temperature and stirred for 4 h then quenched with cold saturated aqueous NaHCO₃ solution. The mixture was extracted with DCM (3×10 mL). The combined organic layers were dried over anhydrous Na₂SO₄, filtered, and concentrated under reduced pressure. The residue was purified by column chromatography (silica gel; 0-100% EtOAc/n-hexane) to provide tert-butyl 4-(3-(2,4-dioxotetrahydropyrimidin-1(2H)-yl)-1-methyl-1H-indazol-6-yl)-4-fluoropiperidine-1-carboxylate (40.0 mg, 40.0% yield) as a white solid. LC/MS m/z [M+H]⁺=446.3. ¹H NMR (400 MHz, (CD₃)₂SO) δ 10.57 (s, 1H), 7.70-7.62 (m, 2H), 7.25-7.18 (m, 1H), 4.10-3.98 (m, 5H), 3.92 (t, 2H), 3.16-3.04 (m, 2H), 2.76 (t, 2H), 2.22-1.92 (m, 4H), 1.44 (s, 9H).

### Step 3. Preparation of 1-(6-(4-fluoro-1-(2-(methylamino)ethyl)piperidin-4-yl)-1-methyl-1H-indazol-3-yl)dihydropyrimidine-2,4(1H,3H)-dione bis(2,2,2-trifluoroacetate)

At 0-10 °C, to a solution of tert-butyl 4-(3-(2,4-dioxotetrahydropyrimidin-1(2H)-yl)-1-methyl-1H-indazol-6-yl)-4-fluoropiperidine-1-carboxylate (0.400 g, 0.898 mmol) in DCM (3 mL) was added TFA (3 mL). The reaction mixture was stirred at room temperature for 1.5 h. The mixture was concentrated in vacuo and lyophilized. The resulting residue was treated with MTBE (20 mL) and stirred for 1 h. The suspension was filtered and the filter cake was collected to afford 1-(6-(4-fluoro-1-(2-(methylamino)ethyl)piperidin-4-yl)-1-methyl-1H-indazol-3-yl)dihydropyrimidine-2,4(1H,3H)-dione bis(2,2,2-trifluoroacetate) (0.640 g, crude) as a solid. The solid was used directly into the next step without further purification. LC/MS m/z [M+H]⁺=446.3. ¹H NMR (400 MHz, (CD₃)₂SO) δ 10.58 (s, 1H), 8.89-8.74 (m, 1H), 8.60-8.52 (m, 1H), 7.80-7.59 (m, 2H), 7.25-7.08 (m, 1H), 4.05-4.01 (m, 4H), 3.93 (t, 3H), 3.46-3.35 (m, 2H), 3.29-3.14 (m, 2H), 2.76 (t, 2H), 2.45-2.12 (m, 4H)(product peaks only).

### Preparation F25

### 1-(6-(4-Fluoro-1-(2-(methylamino)ethyl)piperidin-4-yl)-1-methyl-1H-indazol-3-yl)dihydropyrimidine-2,4(1H,3H)-dione bis(2,2,2-trifluoroacetate)

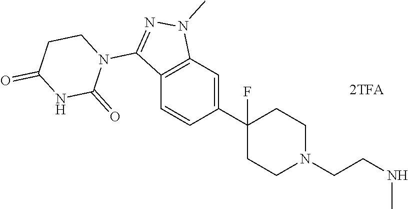 [SMILES: CNCCN1CCC(F)(c2ccc3c(N4CCC(=O)NC4=O)nn(C)c3c2)CC1]

Made in the manner of F6 from Preparation F24. LC/MS m/z [M+H]⁺=403.1. ¹H NMR (400 MHz, (CD₃)₂SO) δ 10.60 (s, 1H), 8.84-8.80 (m, 2H), 7.76-7.62 (m, 2H), 7.16 (d, 1H), 4.05-3.97 (m, 3H), 3.93 (t, 2H), 3.69-3.20 (m, 7H), 2.76 (t, 2H), 2.69-2.54 (m, 4H), 2.45-2.23 (m, 4H).

### Preparation F26

### 1-(6-(4-Hydroxypiperidin-4-yl)-1-methyl-1H-indazol-3-yl)dihydropyrimidine-2,4(1H,3H)-dione bis(2,2,2-trifluoroacetate)

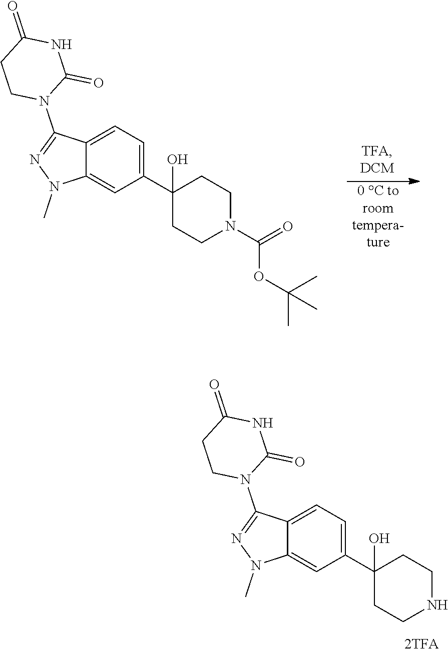 [SMILES: Cn1nc(N2CCC(=O)NC2=O)c2ccc(C3(O)CCN(C(=O)OC(C)(C)C)CC3)cc21 ; Cn1nc(N2CCC(=O)NC2=O)c2ccc(C3(O)CCNCC3)cc21]

### Step 1. Preparation of 1-(6-(4-hydroxypiperidin-4-yl)-1-methyl-1H-indazol-3-yl)dihydropyrimidine-2,4(1H,3H)-dione

At −78 °C, to a solution of tert-butyl 4-(3-(2,4-dioxotetrahydropyrimidin-1(2H)-yl)-1-methyl-1H-indazol-6-yl)-4-hydroxypiperidine-1-carboxylate (0.550 g, 1.24 mmol) in DCM (5 mL) was added TFA (3 mL). The reaction mixture was stirred at room temperature for 2 h. The mixture was concentrated in vacuo. The residue was treated with ACN (5 mL) and water (10 mL) then lyophilized to afford 1-(6-(4-hydroxypiperidin-4-yl)-1-methyl-1H-indazol-3-yl)dihydropyrimidine-2,4(1H,3H)-dione (0.500 g, crude) as a yellow solid. The solid was used directly into the next step without further purification. LC/MS m/z [M+H]⁺=344.1. ¹H NMR (400 MHz, (CD₃)₂SO) δ 10.57 (s, 1H), 8.69-8.62 (m, 1H), 8.46-8.41 (m, 1H), 7.68-7.61 (m, 2H), 7.23-7.16 (m, 1H), 4.02-3.98 (m, 3H), 3.92 (t, 2H), 3.35-3.11 (m, 4H), 2.76 (t, 2H), 2.29-2.16 (m, 2H), 1.82 (d, 2H).

### Preparation F27

### 1-(6-(4-Hydroxy-1-(2-(methylamino)ethyl)piperidin-4-yl)-1-methyl-1H-indazol-3-yl)dihydropyrimidine-2,4(1H,3H)-dione bis(2,2,2-trifluoroacetate)

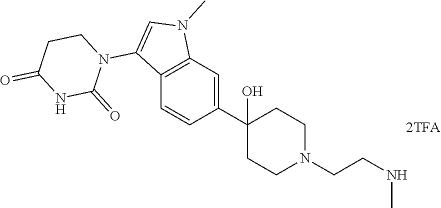 [SMILES: CNCCN1CCC(O)(c2ccc3c(N4CCC(=O)NC4=O)cn(C)c3c2)CC1]

Made in the manner of F6 from Preparation F26. LC/MS m/z [M+H]⁺=401.3. ¹H NMR (400 MHz, (CD₃)₂SO) δ 10.58 (s, 1H), 10.24 (s, 1H), 9.02-8.98 (m, 2H), 7.70-7.61 (m, 2H), 7.23 (d, 1H), 4.02-3.96 (m, 3H), 3.92 (t, 2H), 3.57-3.32 (m, 8H), 2.76 (t, 2H), 2.66 (s, 3H), 2.43-2.32 (m, 2H), 1.91 (d, 2H).

### Preparation F28

### tert-Butyl 5-(((perfluorobutyl)sulfonyl)oxy)-1λ³-oxa-8λ⁴-azatricyclo[5.1.1.0^{3,8}]non-4-ene-8-carboxylate

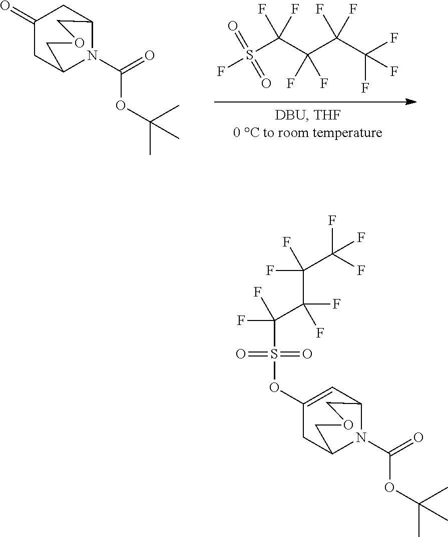 [SMILES: CC(C)(C)OC(=O)N1C2COCC1CC(=O)C2 ; O=S(=O)(F)C(F)(F)C(F)(F)C(F)(F)C(F)(F)F ; CC(C)(C)OC(=O)N1C2C=C(OS(=O)(=O)C(F)(F)C(F)(F)C(F)(F)C(F)(F)F)CC1COC2]

### Step 1. Preparation of tert-butyl 5-(((perfluorobutyl)sulfonyl)oxy)-1 λ³-oxa-8λ⁴-azatricyclo[5.1.1.0^{3,8}]non-4-ene-8-carboxylate

To a mixture of tert-butyl 5-oxo-1λ³-oxa-8λ⁴-azatricyclo[5.1.1.0^{3,8}]nonane-8-carboxylate (CAS: 280761-97-9; 0.500 g, 2.07 mmol) in THF (5 mL) was added DBU (1.89 g, 12.4 mmol). At 0 °C under nitrogen gas, to the reaction mixture was added the solution of nonafluorobutanesulfonyl fluoride (CAS: 375-72-4; 3.76 g, 12.4 mmol) in THF (5 mL) dropwise then stirred at room temperature for 16 h. The residue was diluted with EtOAc (50 mL). The organic layer was washed with water (50 mL), 1 M HCl (30 mL), then brine (2×30 mL). The organic layer was dried over Na₂SO₄ and concentrated by reduced pressure. The residue was purified by column chromatography (silica gel; 0-30% EtOAc:hexane) to afford tert-butyl 5-(((perfluorobutyl)sulfonyl)oxy)-1λ³-oxa-8λ⁴-azatricyclo[5.1.1.0^{3,8}]non-4-ene-8-carboxylate (0.390 g, 36% yield) as a white solid. ¹H NMR (400 MHz, (CD₃)₂SO) δ 6.14-6.07 (m, 1H), 4.58-4.48 (m, 1H), 4.21-4.12 (m, 1H), 3.83 (t, 1H), 3.61-3.48 (m, 3H), 2.73 (dd, 1H), 2.48-2.37 (m, 1H), 1.39 (s, 9H).

### Preparation F29

### 1-(1-Methyl-6-((1R,5S,7s)-9-(2-(methylamino)ethyl)-3-oxa-9-azabicyclo[3.3.1]nonan-7-yl)-1H-indazol-3-yl)dihydropyrimidine-2,4(1H,3H)-dione trifluoroacetate

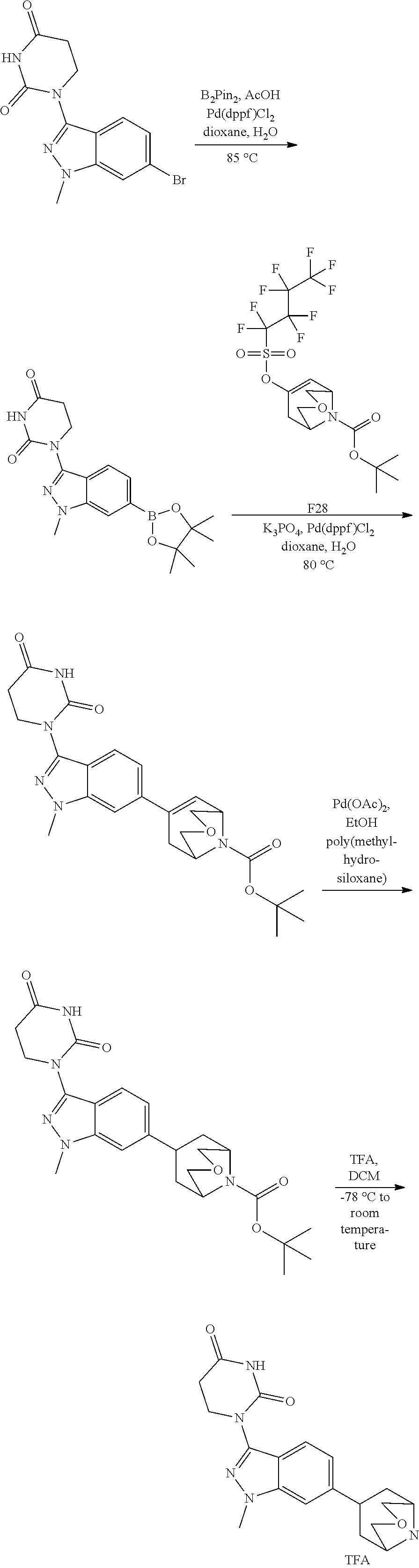

### Step 1. Preparation of 1-(1-methyl-6-(4,4,5,5-tetramethyl-1,3,2-dioxaborolan-2-yl)-1H-indazol-3-yl)dihydropyrimidine-2,4(1H,3H)-dione

A mixture of 1-(6-bromo-1-methyl-1H-indazol-3-yl)dihydropyrimidine-2,4(1H,3H)-dione (CAS: 2446913-88-6; 2.00 g, 6.19 mmol), B₂Pin₂ (2.36 g, 9.28 mmol), and AcOK (1.82 g, 18.6 mmol) in dioxane (20 mL) was degassed with nitrogen gas for 3 min before Pd(dppf)Cl₂ (453 mg, 0.619 mmol) was then added. The reaction mixture was degassed with nitrogen gas for an additional 3 min then stirred at 85 °C for 16 h. After cooling to room temperature, the reaction mixture was filtered through a pad of silica gel. The filter cake was washed with EtOAc. The combined filtrate was concentrated under reduced pressure. The residue was purified by column chromatography (silica gel; 0-80% EtOAc:hexane) to afford 1-(1-methyl-6-(4,4,5,5-tetramethyl-1,3,2-dioxaborolan-2-yl)-1H-indazol-3-yl)dihydropyrimidine-2,4(1H,3H)-dione (2.10 g, 91% yield) as a light solid. LC/MS m/z [M+H]⁺=371.0. ¹H NMR (400 MHz, (CD₃)₂SO) δ 10.58 (s, 1H), 7.90 (s, 1H), 7.66 (d, 1H), 7.38 (d, 1H), 4.06-4.02 (m, 3H), 3.93 (t, 2H), 2.77 (t, 2H), 1.35-1.27 (m, 12H).

### Step 2. Preparation of tert-butyl 5-(3-(2,4-dioxotetrahydropyrimidin-1(2H)-yl)-1-methyl-1H-indazol-6-yl)-1λ³-oxa-8λ⁴-azatricyclo[5.1.1.0^{3,8}]non-4-ene-8-carboxylate

To a stirred solution of 1-(1-methyl-6-(4,4,5,5-tetramethyl-1,3,2-dioxaborolan-2-yl)-1H-indazol-3-yl)dihydropyrimidine-2,4(1H,3H)-dione (0.100 g, 0.270 mmol) and tert-butyl 5-(((perfluorobutyl)sulfonyl)oxy)-1λ³-oxa-8λ⁴-azatricyclo[5.1.1.0^{3,8}]non-4-ene-8-carboxylate (177 mg, 0.338 mmol) in a mixture of dioxane (2 mL) and water (0.2 mL) was added K₃PO₄ (172 mg, 0.810 mmol). The reaction mixture was degassed with nitrogen gas for 3 min, after which Pd(dppf)Cl₂ (24.3 mg, 0.0297 mmol) was added. The suspension was degassed with nitrogen gas for 3 min. The reaction mixture was stirred at 80 °C for 2 h then water (10 mL) was added. The reaction mixture was extracted with EtOAc (3×20 mL). The combined organic layers were washed with water (20 mL) and brine (2×20 mL), dried over Na₂SO₄, and concentrated by reduced pressure. The residue was purified by column chromatography (silica gel; 0-83% EtOAc:hexane) to provide tert-butyl 5-(3-(2,4-dioxotetrahydropyrimidin-1(2H)-yl)-1-methyl-1H-indazol-6-yl)-1λ³-oxa-8λ⁴-azatricyclo[5.1.1.0^{3,8}]non-4-ene-8-carboxylate (0.110 g, 87.1% yield) as a brown solid. LC/MS m/z [M+H]⁺=468.3. ¹H NMR (400 MHz, (CD₃)₂SO) δ 10.55 (s, 1H), 7.64-7.57 (m, 2H), 7.30 (d, 1H), 6.46-6.42 (m, 1H), 4.54-4.49 (m, 1H), 4.25-4.21 (m, 1H), 4.09-3.98 (m, 3H), 3.97-3.83 (m, 3H), 3.67-3.51 (m, 3H), 2.92 (d, 1H), 2.76 (t, 2H), 2.69-2.58 (m, 1H), 1.47-1.36 (m, 9H).

### Step 3. Preparation of tert-butyl 5-(3-(2,4-dioxotetrahydropyrimidin-1(2H)-yl)-1-methyl-1H-indazol-6-yl)-1λ³-oxa-8λ⁴-azatricyclo[5.1.1.0^{3,8}]nonane-8-carboxylate

A mixture of tert-butyl 5-(3-(2,4-dioxotetrahydropyrimidin-1(2H)-yl)-1-methyl-1H-indazol-6-yl)-1λ³-oxa-8λ⁴-azatricyclo[5.1.1.0^{3,8}]non-4-ene-8-carboxylate (50.0 mg, 0.110 mmol) and Pd(OAc)₂ (2.40 mg, 0.0107 mmol) in EtOH (1.0 mL) was protected under nitrogen gas. To this mixture, a solution of poly(methylhydrosiloxane) (64.3 mg, 1.07 mmol) in EtOH (0.5 mL) was added dropwise over 1 min at room temperature. The reaction mixture was stirred at room temperature for 12 h and 20 min then Pd(OAc)₂ (2.40 mg, 0.0107 mmol) and poly(methylhydrosiloxane) (19.3 mg, 0.321 mmol) were added. The reaction mixture was stirred at room temperature for 3 h then filtered and concentrated in vacuo. The residue was purified by column chromatography (silica gel; 0-83% EtOAc:hexane) to provide tert-butyl 5-(3-(2,4-dioxotetrahydropyrimidin-1(2H)-yl)-1-methyl-1H-indazol-6-yl)-1λ³-oxa-8λ⁴-azatricyclo[5.1.1.0^{3,8}]nonane-8-carboxylate as a white solid (20.0 mg, 40.0% yield). LC/MS m/z [M+H]⁺=470.4. ¹H NMR (400 MHz, (CD₃)₂SO) δ 10.55 (s, 1H), 7.57 (d, 1H), 7.45 (s, 1H), 7.03 (d, 1H), 4.22-4.11 (m, 2H), 4.00-3.96 (m, 3H), 3.91 (t, 2H), 3.62 (t, 2H), 3.51-3.43 (m, 3H), 2.79-2.71 (m, 2H), 2.26-2.18 (m, 2H), 1.87-1.76 (m, 2H), 1.49-1.43 (m, 9H).

### Step 4. Preparation of 1-(1-methyl-6-(1λ³-oxa-8-azatricyclo[5.1.1.0^{3,8}]nonan-5-yl)-1H-indazol-3-yl)dihydropyrimidine-2,4(1H,3H)-dione trifluoroacetate

At −78 °C, to a solution of tert-butyl 5-(3-(2,4-dioxotetrahydropyrimidin-1(2H)-yl)-1-methyl-1H-indazol-6-yl)-1λ³-oxa-8λ⁴-azatricyclo[5.1.1.0^{3,8}]nonane-8-carboxylate (0.180 g, 0.383 mmol) in DCM (2.0 mL) was added TFA (2.0 mL). The reaction mixture was stirred at room temperature for 2 h then concentrated under reduced pressure to give an off-white solid. This residue was dissolved in ACN (5 mL) and water (15 mL). The resulting solution was lyophilized to provide 1-(1-methyl-6-(1λ³-oxa-8-azatricyclo[5.1.1.0^{3,8}]nonan-5-yl)-1H-indazol-3-yl)dihydropyrimidine-2,4(1H,3H)-dione trifluoroacetate (0.150 g, crude) as an off-white solid. LC/MS m/z [M+H]⁺=370.1. ¹H NMR (400 MHz, (CD₃)₂SO) δ 10.58 (s, 1H), 9.10-9.06 (m, 1H), 8.60-8.55 (m, 1H), 7.67-7.59 (m, 1H), 7.47 (s, 1H), 7.08-7.01 (m, 1H), 4.09-3.88 (m, 5H), 3.84-3.71 (m, 5H), 3.21-3.09 (m, 1H), 2.76 (t, 2H), 2.38-2.34 (m, 3H), 2.04-1.93 (m, 2H).

### Preparation F30 1-(1-Methyl-6-(8-(2-(methylamino)ethyl)-1λ²-oxa-8λ⁴-azatricyclo[5.1.1.0^{3,8}]nonan-5-yl)-1H-indazol-3-yl)dihydropyrimidine-2,4(1H,3H)-dione bis(2,2,2-trifluoroacetate)

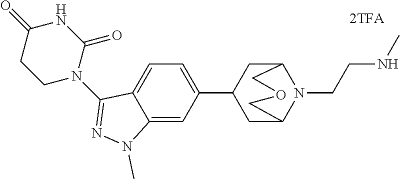 [SMILES: CNCCN1C2COCC1CC(c1ccc3c(N4CCC(=O)NC4=O)nn(C)c3c1)C2]

Synthesized according to Preparation F6 from Preparation F29. LC/MS m/z [M+H]⁺=427.3.

¹H NMR (400 MHz, (CD₃)₂SO) δ 10.55 (s, 1H), 8.19-8.14 (m, 2H), 7.60-7.54 (m, 1H), 7.45-7.41 (m, 1H), 7.13-7.07 (m, 1H), 3.98 (s, 3H), 3.91 (t, 2H), 3.86-3.79 (m, 2H), 3.29-3.22 (m, 2H), 3.04-2.87 (m, 6H), 2.75 (t, 3H), 2.66-2.59 (m, 3H), 2.35-2.14 (m, 2H), 1.88-1.76 (m, 2H).

### Preparation F31

### rac-(R)-3-(4-(1-(2-(Methylamino)ethyl)piperidin-4-yl)phenoxy)piperidine-2,6-dione bis(2,2,2-trifluoroacetate)

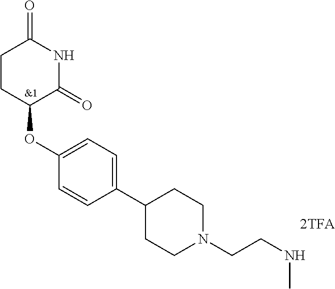 [SMILES: CNCCN1CCC(c2ccc(O[C@H]3CCC(=O)NC3=O)cc2)CC1]

Synthesized according to Preparation F6 from rac-(R)-3-(4-(piperidin-4-yl)phenoxy)piperidine-2,6-dione trifluoroacetate, known in literature from Guckian, K.; et al. Imidazopyridine Compounds for Targeting Degradation of IRAK4 Proteins. World Intellectual Property Organization, Patent Application WO2023283610 A1, Jan. 12, 2023. LC/MS m/z [M+H]⁺=346.1.

### Preparation F32

### rac-(R)-3-((5-Fluoro-2-methoxy-4-(1-((4,5,6,7-tetrahydropyrazolo[1,5-a]pyrazin-2-yl)methyl)piperidin-4-yl)phenyl)amino)piperidine-2,6-dione trifluoroacetate

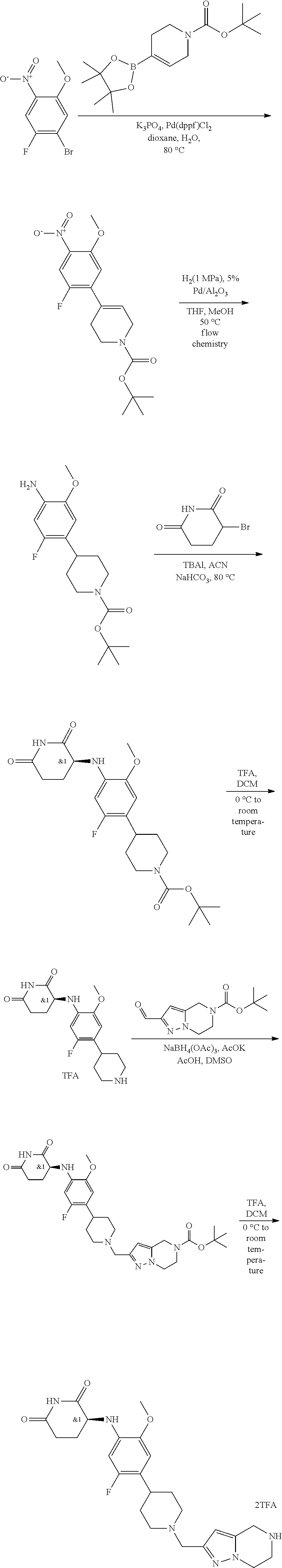

### Step 1. Preparation of tert-butyl 4-(2-fluoro-5-methoxy-4-nitrophenyl)-3,6-dihydropyridine-1(2H)-carboxylate

The same reaction was conducted in two batches then combined for purification. A reaction mixture of tert-butyl 4-(4,4,5,5-tetramethyl-1,3,2-dioxaborolan-2-yl)-3,6-dihydropyridine-1(2H)-carboxylate (CAS: 286961-14-6; 5.19 g, 16.8 mmol), 1-bromo-2-fluoro-5-methoxy-4-nitrobenzene (CAS: 330794-02-0; 4.00 g, 16.0 mmol) in dioxane (40 mL) and H₂O (10 mL) was added K₃PO₄ (8.49 g, 40.0 mmol) to form the first batch. To the suspension of the first batch was added Pd(dppf)Cl₂ (1.17 g, 1.60 mmol) under nitrogen gas. The mixture of the first batch was stirred at 60 °C for 4 h. The second batch of the same reaction was conducted with 1-bromo-2-fluoro-5-methoxy-4-nitrobenzene (CAS: 330794-02-0; 1.00 g, 4.00 mmol). The two batches were combined then added to H₂O (200 mL) and extracted with EtOAc (200 mL×3). The combined organic phase was washed with brine (100 mL×2) to give a black solid. The solid was purified by column chromatography (silica gel; 0-30% EtOAc: DCM) give tert-butyl 4-(2-fluoro-5-methoxy-4-nitrophenyl)-3,6-dihydropyridine-1(2H)-carboxylate (6.51 g, 92.3% yield) as a gray solid. LC/MS m/z [M-tert-butyl+H]⁺=297.0. ¹H NMR (400 MHz, (CD₃)₂SO) δ 7.88 (d, 1H), 7.25 (d, 1H), 6.18 (s, 1H), 4.07-4.01 (m, 2H), 3.94 (s, 3H), 3.54 (t, 2H), 2.53-2.43 (m, 2H), 1.43 (s, 9H).

### Step 2. Preparation of tert-butyl 4-(4-amino-2-fluoro-5-methoxyphenyl)piperidine-1-carboxylate

The same reaction was conducted in two batches then combined for purification. A solution of tert-butyl 4-(2-fluoro-5-methoxy-4-nitrophenyl)-3,6-dihydropyridine-1(2H)-carboxylate (6.00 g, 17.0 mmol) in MeOH (60 mL) and THF (60 mL) was prepared and pumped at a rate of 0.3 mL/min through a fixed-bed stainless steel flow reactor (5.0 mL, 6.35 mm diameter) packed with granular 5% Pd/Al₂O₃ (3.20 g) under a hydrogen atmosphere with a back pressure of 1 MPa and a hydrogen gas flow rate of 30.4 sccm at 50 °C to form the first batch. The reaction mixture of the first batch was continuously collected from the reactor outlet and concentrated under reduced pressure to afford a yellow solid.

A second batch of the same flow reaction was conducted with tert-butyl 4-(2-fluoro-5-methoxy-4-nitrophenyl)-3,6-dihydropyridine-1(2H)-carboxylate (0.500 g, 1.42 mmol). The two batches were combined then diluted with EtOAc (30 mL) and n-hexane (50 mL). The resulting suspension was stirred at room temperature for 30 min which caused precipitation to occur. The precipitate was collected by filtration, washed with EtOAc (20 mL), and dried under high vacuum to provide tert-butyl 4-(4-amino-2-fluoro-5-methoxyphenyl)piperidine-1-carboxylate (4.60 g, crude) as a white solid. The solid was used directly into the next step without further purification. LC/MS m/z [M-tert-butyl+H]⁺=269.1. ¹H NMR (400 MHz, (CD₃)₂SO) δ 6.63 (d, 1H), 6.37 (d, 1H), 4.83 (s, 2H), 4.09-4.02 (m, 2H), 3.73 (s, 3H), 2.83-2.72 (m, 3H), 1.67-1.44 (m, 4H), 1.41 (s, 9H).

### Step 3. Preparation of rac-tert-butyl (R)-4-(4-((2,6-dioxopiperidin-3-yl)amino)-2-fluoro-5-methoxyphenyl)piperidine-1-carboxylate

The same reaction was conducted in two batches then combined for purification. To a solution of tert-butyl 4-(4-amino-2-fluoro-5-methoxyphenyl)piperidine-1-carboxylate (2.00 g, 6.17 mmol) and 3-bromopiperidine-2,6-dione (2.37 g, 12.3 mmol) in ACN (40 mL) were added NaHCO₃ (1.29 g, 15.4 mmol) and TBAI (342 mg, 0.925 mmol) to form the first batch. The reaction mixture of the first batch was sealed, degassed with nitrogen for 3 min, and heated at 80 °C for 72 h.

A second batch of the same reaction was conducted with tert-butyl 4-(4-amino-2-fluoro-5-methoxyphenyl)piperidine-1-carboxylate (0.200 g, 0.617 mmol). The two batches were combined then filtered. The filter cake was washed with ACN (20 mL). The filtrate was concentrated under reduced pressure to afford a yellow gum. The gum was purified twice by column chromatography (silica gel; gradient: 0-10% MeOH: DCM) then (silica gel; gradient: 0-40% EtOAc: DCM) to afford rac-tert-butyl (R)-4-(4-((2,6-dioxopiperidin-3-yl)amino)-2-fluoro-5-methoxyphenyl)piperidine-1-carboxylate (2.30 g, crude) as a blue gum. The blue gum was suspended in a mixture of EtOAc (30 mL) and n-hexane (60 mL) then stirred at room temperature for 30 min. The suspension was filtered then the filter cake was washed with EtOAc (10 mL). The filter cake was collected then dried under high vacuum to give a blue solid. The blue solid was suspended in MTBE (60 mL) and stirred at room temperature for 30 min. The suspension was filtered. The filter cake was washed with MTBE (20 mL) then collected and dried under high vacuum to give rac-tert-butyl (R)-4-(4-((2,6-dioxopiperidin-3-yl)amino)-2-fluoro-5-methoxyphenyl)piperidine-1-carboxylate (0.760 g, 25.8% yield) as a blue solid. LC/MS m/z [M-tert-butyl+H]⁺=380.1. ¹H NMR (400 MHz, (CD₃)₂SO) δ 10.86 (s, 1H), 6.70 (d, 1H), 6.48 (d, 1H), 5.28 (d, 1H), 4.34-4.24 (m, 1H), 4.10-3.98 (m, 2H), 3.78 (s, 3H), 2.88-2.73 (m, 4H), 2.62-2.54 (m, 1H), 2.17-2.07 (m, 1H), 2.01-1.86 (m, 1H), 1.68-1.49 (m, 4H), 1.41 (s, 9H).

### Step 4. Preparation of rac-(R)-3-((5-fluoro-2-methoxy-4-(piperidin-4-yl)phenyl)amino)piperidine-2,6-dione

To a stirred solution of rac-tert-butyl (R)-4-(4-((2,6-dioxopiperidin-3-yl)amino)-2-fluoro-5-methoxyphenyl)piperidine-1-carboxylate (0.760 g, 1.75 mmol) in DCM (7.6 mL) was added TFA (7.6 mL) at 0 °C. The reaction mixture was warmed to room temperature and stirred for 3 h. The mixture was concentrated under reduced pressure to afford a crude blue gum, which was lyophilized to give rac-(R)-3-((5-fluoro-2-methoxy-4-(piperidin-4-yl)phenyl)amino)piperidine-2,6-dione (0.930 g, crude) as a blue solid. The solid was used directly into the next step without further purification. LC/MS m/z [M+H]⁺=336.1. ¹H NMR (400 MHz, (CD₃)₂SO) δ 10.88 (s, 1H), 8.78 (d, 1H), 8.55-8.48 (m, 1H), 6.64 (d, 1H), 6.52 (d, 1H), 4.32 (dd, 1H), 3.80 (s, 3H), 3.40-3.36 (m, 1H), 3.05-2.93 (m, 3H), 2.87-2.74 (m, 1H), 2.60-2.52 (m, 2H), 2.17-2.05 (m, 1H), 2.05-1.78 (m, 5H).

### Step 5. Preparation of rac-tert-butyl (R)-2-((4-(4-((2,6-dioxopiperidin-3-yl)amino)-2-fluoro-5-methoxyphenyl)piperidin-1-yl)methyl)-6,7-dihydropyrazolo[1,5-a]pyrazine-5(4H)-carboxylate

The same reaction was conducted in two batches then combined for purification. To a solution of rac-(R)-3-((5-fluoro-2-methoxy-4-(piperidin-4-yl)phenyl)amino)piperidine-2,6-dione (0.400 g, 0.710 mmol) and tert-butyl 2-formyl-6,7-dihydropyrazolo[1,5-a]pyrazine-5(4H)-carboxylate (CAS: 1286754-11-7; 0.178 g, 0.710 mmol) in DMSO (4 mL) were added AcOK (0.139 g, 1.42 mmol), AcOH (42.6 mg, 0.710 mmol) then NaBH(OAc)₃ (0.301 g, 1.42 mmol) to form the first batch. The mixture of the first batch was stirred at room temperature under nitrogen gas for 5 h.

The second batch of the same reaction was conducted with rac-(R)-3-((5-fluoro-2-methoxy-4-(piperidin-4-yl)phenyl)amino)piperidine-2,6-dione (0.100 g, 0.178 mmol). The two batches were combined then poured into H₂O (20 mL) and extracted with EtOAc (30 mL×3). The combined organic layers were washed with brine (30 mL×2). The organic layer was dried over Na₂SO₄ and concentrated in vacuo to get a blue solid. The blue solid was diluted with EtOAc (5 mL) and n-hexane (6 mL). The suspension was stirred at room temperature for 30 min, which caused precipitation to form. The mixture was filtered. The filter cake was washed with EtOAc (10 mL) then collected and dried under high vacuum to give rac-tert-butyl (R)-2-((4-(4-((2,6-dioxopiperidin-3-yl)amino)-2-fluoro-5-methoxyphenyl)piperidin-1-yl)methyl)-6,7-dihydropyrazolo[1,5-a]pyrazine-5(4H)-carboxylate (0.430 g, crude) as a blue solid. The solid was used directly into the next step without further purification. ¹H NMR (400 MHz, (CD₃)₂SO) δ 10.85 (s, 1H), 10.20-10.15 (m, 1H), 6.61 (d, 1H), 6.51 (d, 1H), 6.39 (s, 1H), 5.35 (d, 1H), 4.62 (s, 2H), 4.36-4.20 (m, 3H), 4.13 (t, 2H), 3.82 (t, 2H), 3.78 (s, 3H), 3.47 (d, 2H), 3.17-3.00 (m, 2H), 2.98-2.72 (m, 2H), 2.55 (d, 2H), 2.17-2.01 (m, 2H), 1.94-1.83 (m, 3H), 1.44 (s, 9H).

### Step 6. Preparation of rac-(R)-3-((5-fluoro-2-methoxy-4-(1-((4,5,6,7-tetrahydropyrazolo[1,5-a]pyrazin-2-yl)methyl)piperidin-4-yl)phenyl)amino)piperidine-2,6-dione trifluoroacetate

To a solution of rac-tert-butyl (R)-2-((4-(4-((2,6-dioxopiperidin-3-yl)amino)-2-fluoro-5-methoxyphenyl)piperidin-1-yl)methyl)-6,7-dihydropyrazolo[1,5-a]pyrazine-5(4H)-carboxylate (0.430 g, 0.754 mmol) in DCM (4.3 mL) was added TFA (4.3 mL) at 0 °C. The reaction mixture was warmed to room temperature and stirred for 30 min. The mixture was concentrated under reduced pressure and lyophilized to give rac-(R)-3-((5-fluoro-2-methoxy-4-(1-((4,5,6,7-tetrahydropyrazolo[1,5-a]pyrazin-2-yl)methyl)piperidin-4-yl)phenyl)amino)piperidine-2,6-dione trifluoroacetate (0.380 g, crude) as a blue solid. The solid was used directly into the next step without further purification. LC/MS m/z [M+H]⁺=471.3. ¹H NMR (400 MHz, (CD₃)₂SO) δ 10.86 (s, 1H), 10.31 (s, 1H), 9.92 (s, 2H), 6.63 (d, 1H), 6.59-6.39 (m, 2H), 4.47 (s, 2H), 4.38-4.27 (m, 5H), 3.77 (s, 3H), 3.70 (t, 2H), 3.47 (d, 2H), 3.15-3.04 (m, 2H), 2.99-2.88 (m, 1H), 2.86-2.72 (m, 1H), 2.59-2.52 (m, 1H), 2.20-1.84 (m, 6H).

### Preparation F33 7-Chloro-3-iodo-1-methyl-1H-pyrazolo[4,3-b]pyridine

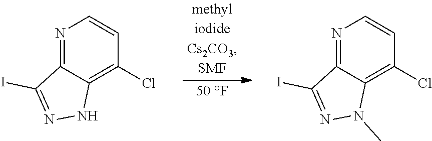

Step 1. Preparation of 7-chloro-3-iodo-1-methyl-1H-pyrazolo[4,3-b]pyridine

The reaction was conducted in two batches then combined for purification. To a stirred solution of 7-chloro-3-iodo-1H-pyrazolo[4,3-b]pyridine (CAS: 1357945-92-6; 4.92 g, 17.6 mmol) in DMF (50 mL) was added Cs₂CO₃ (14.3 g, 44.0 mmol) and methyl iodide (3.00 g, 21.1 mmol) at 0 °C under nitrogen gas to form the first batch. The reaction mixture of the first batch was stirred at 50 °C for 1 h.

A second batch of the same reaction was conducted with 7-chloro-3-iodo-1H-pyrazolo[4,3-b]pyridine (CAS: 1357945-92-6; 2.10 g, 7.51 mmol). The two batches were combined then cooled to 0 °C then quenched with H₂O (500 mL). The suspension was filtered and the filter cake was washed with H₂O (100 mL) then collected and lyophilized to give 7-chloro-3-iodo-1-methyl-1H-pyrazolo[4,3-b]pyridine (5.93 g, 80.5% yield). LC/MS m/z [M+H]⁺=293.8. ¹H NMR (400 MHz, (CD₃)₂SO) δ 8.50-8.43 (m, 1H), 7.69-7.63 (m, 1H), 4.32 (s, 3H).

### Preparation F34

### 1-(7-Chloro-1-methyl-1H-pyrazolo[4,3-b]pyridin-3-yl)-3-(4-methoxybenzyl)dihydropyrimidine-2,4(1H,3H)-dione

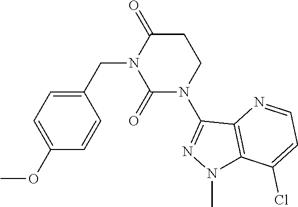 [SMILES: COc1ccc(CN2C(=O)CCN(c3nn(C)c4c(Cl)ccnc34)C2=O)cc1]

Synthesized according to Preparation F11 with Preparation F33. The reaction was altered by stirring at 100 °C for 16 h then quenched with H₂O (600 mL) and extracted with EtOAc (500 mL). The combined organic layer was washed with brine (2×300 mL) then concentrated in vacuo. The residue was purified by column chromatography (silica gel; 0-12% EtOAc: DCM) to provide 1-(7-chloro-1-methyl-1H-pyrazolo[4,3-b]pyridin-3-yl)-3-(4-methoxybenzyl)dihydropyrimidine-2,4(1H,3H)-dione (3.40 g, 46.0% yield) as a yellow solid. LC/MS m/z [M+H]⁺=400.1. ¹H NMR (400 MHz, (CD₃)₂SO) δ 8.45 (d, 1H), 7.63 (d, 1H), 7.27-7.14 (m, 2H), 6.90-6.81 (m, 2H), 4.82 (s, 2H), 4.29 (s, 3H), 3.90 (t, 2H), 3.75-3.69 (m, 3H), 2.95 (t, 2H).

### Preparation F35

### 1-(1-Methyl-7-(4-(2-(methylamino)ethyl)piperazin-1-yl)-1H-pyrazolo[4,3-b]pyridin-3-yl)dihydropyrimidine-2,4(1H,3H)-dione

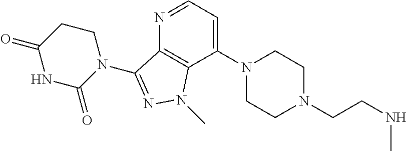 [SMILES: CNCCN1CCN(c2ccnc3c(N4CCC(=O)NC4=O)nn(C)c23)CC1]

Synthesized according to Preparation F12 with Preparation F34. LC/MS m/z [M+H]⁺=387.2.

### Preparation F36

### 7-Chloro-3-iodo-1-methyl-1H-pyrazolo[4,3-c]pyridine

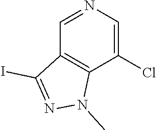

Synthesized according to Preparation F33 with 7-chloro-3-iodo-1H-pyrazolo[4,3-c]pyridine (CAS: 1357946-98-5). LC/MS m/z [M+H]⁺=293.8. ¹H NMR (400 MHz, (CD₃)₂SO) δ 8.66 (s, 1H), 8.42 (s, 1H), 4.28 (s, 3H).

### Preparation F37

### 1-(7-Chloro-1-methyl-1H-pyrazolo[4,3-c]pyridin-3-yl)-3-(4-methoxybenzyl)dihydropyrimidine-2,4(1H,3H)-dione

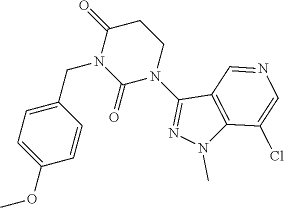 [SMILES: COc1ccc(CN2C(=O)CCN(c3nn(C)c4c(Cl)cncc34)C2=O)cc1]

Synthesized according to Preparation F11 with Preparation F36. The reaction was altered by being quenched with H₂O (60 mL) and extracted with EtOAc (60 mL×3). The combined organic layer was washed with brine (2×30 mL) then concentrated in vacuo. The residue was purified by column chromatography (silica gel; 0-10% MeOH: DCM) to provide 1-(7-chloro-1-methyl-1H-pyrazolo[4,3-c]pyridin-3-yl)-3-(4-methoxybenzyl)dihydropyrimidine-2,4(1H,3H)-dione (2.90 g, 50.1% yield) as a yellow solid. LC/MS m/z [M+H]⁺=400.0. ¹H NMR (400 MHz, CDCl₃) δ 9.01 (s, 1H), 8.39-8.35 (m, 1H), 7.49-7.41 (m, 2H), 6.90-6.81 (m, 2H), 5.03 (s, 2H), 4.31 (s, 3H), 4.07 (t, 2H), 3.80 (s, 3H), 3.00-2.92 (m, 2H).

### Preparation F38

### 1-(1-Methyl-7-(4-(2-(methylamino)ethyl)piperazin-1-yl)-1H-pyrazolo[4,3-c]pyridin-3-yl)dihydropyrimidine-2,4(1H,3H)-dione

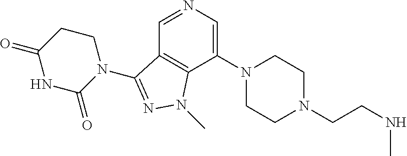 [SMILES: CNCCN1CCN(c2cncc3c(N4CCC(=O)NC4=O)nn(C)c23)CC1]

Synthesized according to Preparation F12 with Preparation F37. LC/MS m/z [M+H]⁺=387.2.

### Preparation F39

### rac-(R)-3-(3-Fluoro-4-(4-(2-(methylamino)ethyl)piperidin-1-yl)phenyl)piperidine-2,6-dione bis(2,2,2-trifluoroacetate)

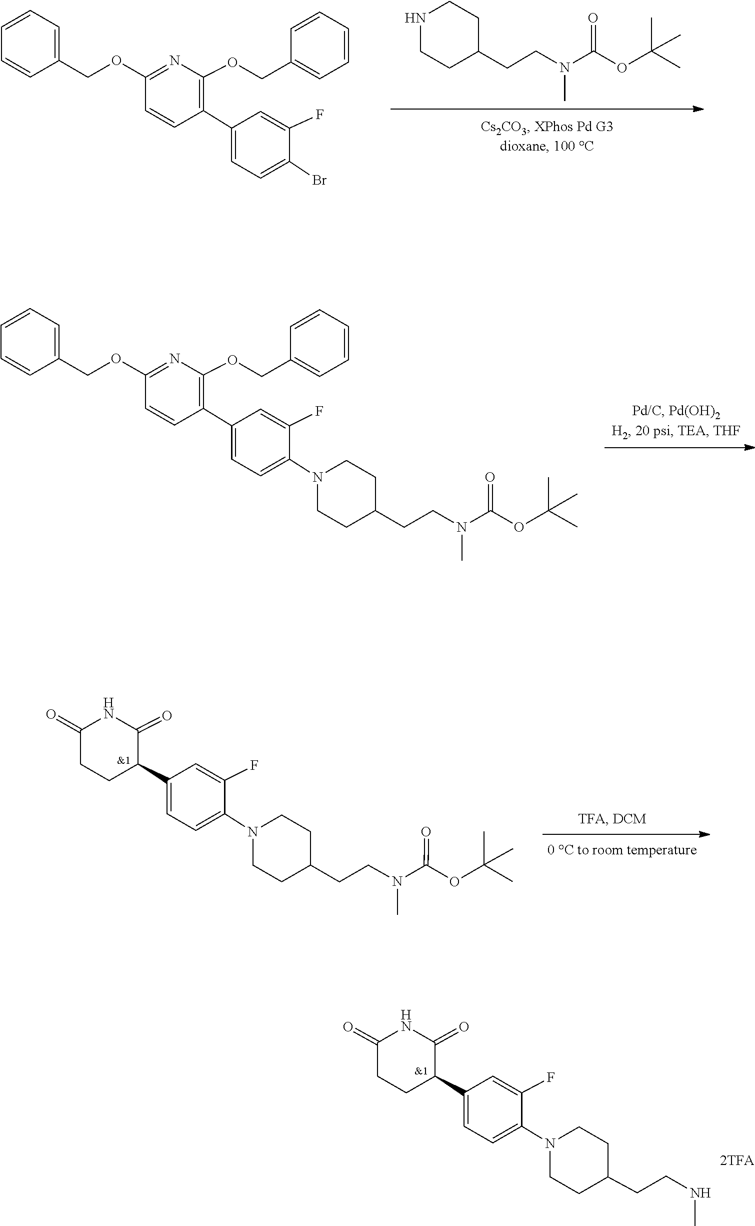 [SMILES: Fc1cc(-c2ccc(OCc3ccccc3)nc2OCc2ccccc2)ccc1Br ; CN(CCC1CCNCC1)C(=O)OC(C)(C)C ; CN(CCC1CCN(c2ccc(-c3ccc(OCc4ccccc4)nc3OCc3ccccc3)cc2F)CC1)C(=O)OC(C)(C)C ; CN(CCC1CCN(c2ccc([C@H]3CCC(=O)NC3=O)cc2F)CC1)C(=O)OC(C)(C)C ; CNCCC1CCN(c2ccc([C@H]3CCC(=O)NC3=O)cc2F)CC1]

### Step 1. Preparation of tert-butyl(2-(1-(4-(2,6-bis(benzyloxy)pyridin-3-yl)-2-fluorophenyl)piperidin-4-yl)ethyl)(methyl)carbamate

To a mixture of 2,6-bis(benzyloxy)-3-(4-bromo-3-fluorophenyl)pyridine (CAS: 2902652-28-0; 0.400 g, 0.861 mmol) and tert-butyl methyl(2-(piperidin-4-yl)ethyl)carbamate (261 mg, 1.08 mmol) in dioxane (8.0 mL) was added Cs₂CO₃ (842 mg, 2.58 mmol), then degassed with nitrogen for 3 min. XPhos Pd G3 (72.9 mg, 0.0861 mmol) was added under a nitrogen atmosphere then the reaction mixture was degassed with nitrogen gas for 3 min. The reaction mixture was stirred at 100 °C for 16 h. After cooling, the reaction mixture was concentrated in vacuo, diluted with H₂O (10 mL), then extracted with EtOAc (3×10 mL). The combined organic layers were washed with brine (2×25 mL), dried over Na₂SO₄, and concentrated under reduced pressure. The residue was purified by column chromatography (silica gel; 0-7% EtOAc: petroleum ether) to afford tert-butyl(2-(1-(4-(2,6-bis(benzyloxy)pyridin-3-yl)-2-fluorophenyl)piperidin-4-yl)ethyl)(methyl)carbamate (0.390 g, 72.3% yield) as a colorless gum. LC/MS m/z [M+H]⁺=626.4. ¹H NMR (400 MHz, (CD₃)₂SO) δ 7.75 (d, 1H), 7.49-7.26 (m, 12H), 7.02 (t, 1H), 6.53 (d, 1H), 5.41 (s, 2H), 5.36 (s, 2H), 3.40-3.33 (m, 2H), 3.22 (t, 2H), 2.76 (s, 3H), 2.70-2.58 (m, 2H), 1.82-1.75 (m, 2H), 1.46-1.42 (m, 2H), 1.40 (s, 9H), 1.34-1.29 (m, 3H).

### Step 2. Preparation of rac-tert-butyl (R)-(2-(1-(4-(2,6-dioxopiperidin-3-yl)-2-fluorophenyl)piperidin-4-yl)ethyl)(methyl)carbamate

To a solution of tert-butyl(2-(1-(4-(2,6-bis(benzyloxy)pyridin-3-yl)-2-fluorophenyl)piperidin-4-yl)ethyl)(methyl)carbamate (0.390 g, 0.623 mmol) in THF (5 mL) was added TEA (63.1 mg, 0.623 mmol), followed by the addition of 10% Pd/C (66.3 mg, 0.0623 mmol) and 10% Pd(OH)₂/C (87.5 mg, 0.0623 mmol) under an argon atmosphere. The reaction mixture was stirred under hydrogen gas at room temperature and 20 psi for 16 h. The mixture was then filtered through a pad of Celite, and the filtrate was concentrated under reduced pressure. The residue was purified by column chromatography (silica gel; 0-70% EtOAc: petroleum ether) to afford rac-tert-butyl (R)-(2-(1-(4-(2,6-dioxopiperidin-3-yl)-2-fluorophenyl)piperidin-4-yl)ethyl)(methyl)carbamate (203 mg, 72.8% yield) as a colorless gum. LC/MS m/z [M+H]⁺=448.4. ¹H NMR (400 MHz, (CD₃)₂SO) δ 10.80 (s, 1H), 7.04-6.90 (m, 3H), 3.79 (dd, 1H), 3.36-3.32 (m, 1H), 3.22 (t, 2H), 2.76 (s, 3H), 2.71-2.55 (m, 3H), 2.49-2.43 (m, 1H), 2.26-2.11 (m, 1H), 2.05-1.95 (m, 1H), 1.81-1.74 (m, 2H), 1.46-1.42 (m, 2H), 1.39 (s, 9H), 1.33-1.29 (m, 4H).

### Step 3. Preparation of rac-(R)-3-(3-fluoro-4-(4-(2-(methylamino)ethyl)piperidin-1-yl)phenyl)piperidine-2,6-dione bis(2,2,2-trifluoroacetate)

To a solution of rac-tert-butyl (R)-(2-(1-(4-(2,6-dioxopiperidin-3-yl)-2-fluorophenyl)piperidin-4-yl)ethyl)(methyl)carbamate (203 mg, 0.454 mmol) in DCM (4.0 mL) was added TFA (2.0 mL) at 0-5 °C. The reaction mixture was stirred at room temperature for 2 h, then concentrated under reduced pressure. The residue was dissolved in distilled H₂O (20 mL) and lyophilized to afford rac-(R)-3-(3-fluoro-4-(4-(2-(methylamino)ethyl)piperidin-1-yl)phenyl)piperidine-2,6-dione bis(2,2,2-trifluoroacetate) (268 mg, crude) as a colorless solid. The solid was used directly into the next step without further purification. LC/MS m/z [M+H]⁺=348.3. ¹H NMR (400 MHz, (CD₃)₂SO) δ 10.80 (s, 1H), 8.35 (s, 2H), 7.05-6.91 (m, 3H), 3.79 (dd, 1H), 3.37-3.29 (m, 2H), 3.01-2.89 (m, 2H), 2.74-2.53 (m, 6H), 2.49-2.42 (m, 1H), 2.26-2.11 (m, 1H), 2.05-1.95 (m, 1H), 1.79-1.71 (m, 2H), 1.56 (q, 2H), 1.52-1.43 (m, 1H), 1.39-1.21 (m, 2H).

### Preparation F40 rac-(R)-3-((3-Fluoro-4-(4-(2-(methylamino)ethyl)piperazin-1-yl)phenyl)amino)piperidine-2,6-dione bis(2,2,2-trifluoroacetate)

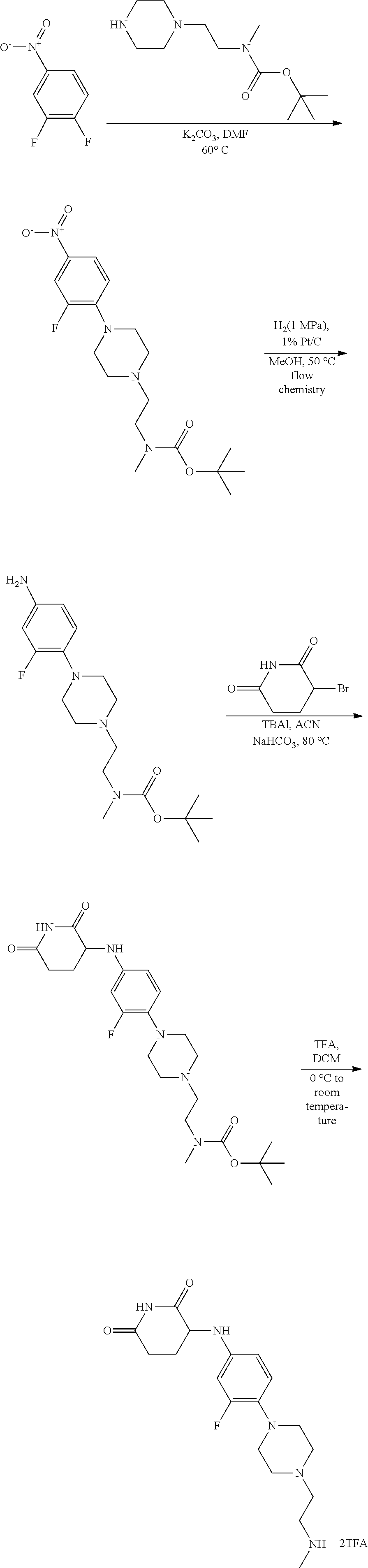

### Step 1. Preparation of tert-butyl(2-(4-(2-fluoro-4-nitrophenyl)piperazin-1-yl)ethyl)(methyl)carbamate

To a solution of 1,2-difluoro-4-nitrobenzene (CAS: 369-34-6; 0.500 g, 2.05 mmol) in DMF (8 mL) was slowly added tert-butyl methyl(2-(piperazin-1-yl)ethyl)carbamate (CAS: 1540928-69-5; 343 mg, 2.16 mmol), followed by the addition of K₂CO₃ (568 mg, 4.11 mmol). The reaction mixture was stirred at 60 °C for 0.5 h, then continued at 60 °C for an additional 3 h. The reaction mixture was diluted with H₂O (25 mL) and extracted with EtOAc (3×25 mL). The combined organic layers were washed with brine (3×70 mL), dried over Na₂SO₄, and concentrated under reduced pressure. The residue was purified by column chromatography (silica gel; 0-100% EtOAc:hexane) to afford tert-butyl(2-(4-(2-fluoro-4-nitrophenyl)piperazin-1-yl)ethyl)(methyl)carbamate (0.630 g, 80.2% yield) as a yellow gum. LC/MS m/z [M+H]⁺=383.2. ¹H NMR (400 MHz, (CD₃)₂SO) δ 8.05-7.97 (m, 2H), 7.18 (t, 1H), 3.33-3.26 (m, 6H), 2.81 (s, 3H), 2.59 (t, 4H), 2.50-2.46 (m, 2H), 1.41 (s, 9H).

### Step 2. Preparation of tert-butyl(2-(4-(4-amino-2-fluorophenyl)piperazin-1-yl)ethyl)(methyl)carbamate

A solution of tert-butyl(2-(4-(2-fluoro-4-nitrophenyl)piperazin-1-yl)ethyl)(methyl)carbamate (0.630 g, 1.65 mmol) in MeOH (15 mL) was delivered via plunger pump at a flow rate of 0.30 mL/min. Hydrogen gas was introduced via a mass flow controller at 30 sccm. The system was maintained at 1 MPa. The combined liquid and gas streams were mixed with a T-shaped mixer before being passed into a stainless-steel fixed-bed reactor (outer diameter: 6.350 mm (¼ inch), internal volume: 5.00 mL) packed with 1% Pt/C (2.49 g) and held at 50 °C. After complete injection of the reaction solution, the solvent feed was switched to MeOH at 10 mL/min and the reactor was flushed for 10 min. The reaction mixture was concentrated under reduced pressure to afford tert-butyl(2-(4-(4-amino-2-fluorophenyl)piperazin-1-yl)ethyl)(methyl)carbamate (165 mg, 28.4% yield) as a yellow solid. ¹H NMR (400 MHz, (CD₃)₂SO) δ 6.86-6.66 (m, 1H), 6.37-6.25 (m, 2H), 4.95 (s, 2H), 3.31-3.10 (m, 4H), 2.82-2.76 (m, 7H), 2.58-2.51 (m, 2H) 2.45-2.40 (m, 2H), 1.39 (s, 9H).

### Step 3. Preparation of tert-butyl(2-(4-(4-((2,6-dioxopiperidin-3-yl)amino)-2-fluorophenyl)piperazin-1-yl)ethyl)(methyl)carbamate

To a solution of tert-butyl(2-(4-(4-amino-2-fluorophenyl)piperazin-1-yl)ethyl)(methyl)carbamate (165 mg, 0.468 mmol) and 3-bromopiperidine-2,6-dione (0.18 g, 0.94 mmol) in ACN (4 mL) were added NaHCO₃ (98 mg, 1.2 mmol) and TBAI (26 mg, 0.070 mmol). The reaction mixture was sealed, degassed with nitrogen for 3 min, and stirred at 80 °C for 30 h. The resulting mixture was filtered. The filter cake was washed with ACN (20 mL). The filtrate was concentrated under reduced pressure. The residue was purified by column chromatography (silica gel; 0-100% EtOAc: petroleum ether) to provide tert-butyl(2-(4-(4-((2,6-dioxopiperidin-3-yl)amino)-2-fluorophenyl)piperazin-1-yl)ethyl)(methyl)carbamate (96 mg, 44% yield) as a yellow gum. LC/MS m/z [M+H]⁺=464.3. ¹H NMR (400 MHz, (CD₃)₂SO) δ (ppm): 10.77 (s, 1H), 6.82 (t, 1H), 6.51 (dd, 1H), 6.42 (dd, 1H), 5.80 (d, 1H), 4.31-4.21 (m, 1H), 3.30-3.24 (m, 3H), 2.94-2.66 (m, 8H), 2.60-2.53 (m, 3H), 2.49-2.38 (m, 2H), 2.14-2.05 (m, 1H), 1.94-1.78 (m, 1H), 1.40 (s, 10H).

### Step 4. Preparation of rac-(R)-3-((3-fluoro-4-(4-(2-(methylamino)ethyl)piperazin-1-yl)phenyl)amino)piperidine-2,6-dione bis(2,2,2-trifluoroacetate)

To a solution of tert-butyl(2-(4-(4-((2,6-dioxopiperidin-3-yl)amino)-2-fluorophenyl)piperazin-1-yl)ethyl)(methyl)carbamate (96.0 mg, 0.210 mmol) in DCM (2 mL) was added TFA (1 mL) at 0-5 °C. The reaction mixture was stirred at room temperature for 2 h then concentrated under reduced pressure to afford a brown gum. The residue was dissolved in distilled H₂O (20 mL) and lyophilized to provide rac-(R)-3-((3-fluoro-4-(4-(2-(methylamino)ethyl)piperazin-1-yl)phenyl)amino)piperidine-2,6-dione bis(2,2,2-trifluoroacetate) (0.120 g, crude) as a black solid. The solid was used directly in the next step without further purification. LC/MS m/z [M+H]⁺=364.3.

### Preparation F41

### rac-(R)-3-(1-Methyl-6-((4-(2-(methylamino)ethyl)piperidin-1-yl)methyl)-1H-indazol-3-yl)piperidine-2,6-dione bis(2,2,2-trifluoroacetate)

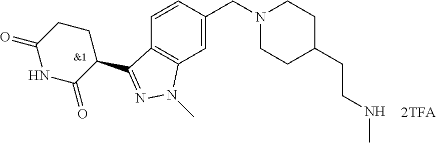 [SMILES: CNCCC1CCN(Cc2ccc3c([C@@H]4CCC(=O)NC4=O)nn(C)c3c2)CC1]

Synthesized according to Preparation F3 with CAS: 2925099-25-6. LC/MS m/z [M+H]⁺=398.3. ¹H NMR (400 MHz, (CD₃)₂SO) δ 10.93 (s, 1H), 9.89-9.84 (m, 1H), 8.49-8.44 (m, 2H), 7.85-7.74 (m, 2H), 7.30-7.21 (m, 1H), 4.49-4.37 (m, 2H), 4.02 (s, 3H), 3.40 (d, 2H), 2.96-2.82 (m, 5H), 2.78-2.57 (m, 2H), 2.60-2.51 (m, 3H), 2.46-2.32 (m, 1H), 2.22-2.13 (m, 1H), 1.84 (d, 2H), 1.77-1.63 (m, 1H), 1.61-1.44 (m, 2H), 1.44-1.27 (m, 2H).

### Preparation F42

### rac-(R)-3-((2-Methoxy-4-(1-(2-(methylamino)ethyl)piperidin-4-yl)phenyl)amino)piperidine-2,6-dione bis(2,2,2-trifluoroacetate)

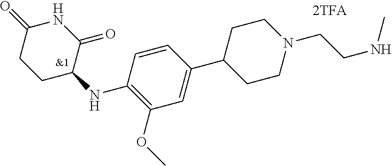 [SMILES: CNCCN1CCC(c2ccc(N[C@H]3CCC(=O)NC3=O)c(OC)c2)CC1]

Synthesized according to Step 3 to 6 from Preparation F32 with CAS: 1089280-53-4. The S_{N}Ar conditions in step 3 were altered to not utilize TBAI and the stir occurred at 100 °C. Step 5 utilized CAS: 123387-72-4. LC/MS m/z [M+H]⁺=375.3. ¹H NMR (400 MHz, (CD₃)₂SO) δ 10.87 (s, 1H), 8.83 (s, 2H), 6.73 (s, 1H), 6.68-6.57 (m, 2H), 4.26 (dd, 1H), 3.81 (s, 3H), 3.66-3.59 (m, 2H), 3.39 (s, 4H), 3.11-3.07 (m, 2H), 2.86-2.70 (m, 1H), 2.65 (s, 3H), 2.21-2.11 (m, 2H), 2.04-1.80 (m, 6H).

### Preparation F43

### rac-(R)-3-((3-Fluoro-4-(1-(2-(methylamino)ethyl)piperidin-4-yl)phenyl)amino)piperidine-2,6-dione bis(2,2,2-trifluoroacetate)

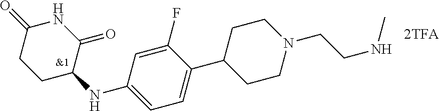 [SMILES: CNCCN1CCC(c2ccc(N[C@H]3CCC(=O)NC3=O)cc2F)CC1]

Synthesized according to Step 3 to 6 from Preparation F32 with CAS:188975-15-7. Step 5 utilized CAS: 123387-72-4. LC/MS m/z [M+H]⁺=363.2. ¹H NMR (400 MHz, (CD₃)₂SO) δ 10.80 (s, 1H), 10.34 (s, 1H), 9.14 (s, 2H), 6.94 (t, 1H), 6.53-6.43 (m, 2H), 4.32 (dd, 1H), 3.63 (d, 2H), 3.42 (s, 4H), 3.14 (t, 2H), 2.95 (t, 1H), 2.80-2.67 (m, 1H), 2.64 (s, 3H), 2.62-2.52 (m, 1H), 2.15-1.76 (m, 6H).

### Preparation F44

### rac-(R)-3-((5-Fluoro-2-methoxy-4-(1-(2-(methylamino)ethyl)piperidin-4-yl)phenyl)amino)piperidine-2,6-dione bis(2,2,2-trifluoroacetate)

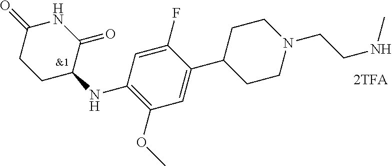 [SMILES: CNCCN1CCC(c2cc(OC)c(N[C@H]3CCC(=O)NC3=O)cc2F)CC1]

Synthesized according to Step 3 to 6 from Preparation F32 with CAS:1807448-53-8 from known in literature from Nasveschuk, C.; et al. Preparation of substituted quinazolinones for the degradation of mutant BRAF. Patent WO2022261250 A1, Step 5 utilized CAS: 123387-72-4. LC/MS m/z [M+H]⁺=393.2. ¹H NMR (400 MHz, (CD₃)₂SO) δ 10.86 (s, 1H), 9.84-9.79 (m, 1H), 8.81-8.76 (m, 2H), 6.63 (d, 1H), 6.52 (d, 1H), 4.32 (dd, 1H), 3.79 (s, 3H), 3.62 (d, 2H), 3.38 (s, 4H), 3.20-2.93 (m, 3H), 2.86-2.72 (m, 1H), 2.65 (s, 3H), 2.16-1.87 (m, 7H).

### Preparation F45

### 3-(2,6-Bis(benzyloxy)pyridin-3-yl)-6-bromo-1-(difluoromethyl)-1H-indazole

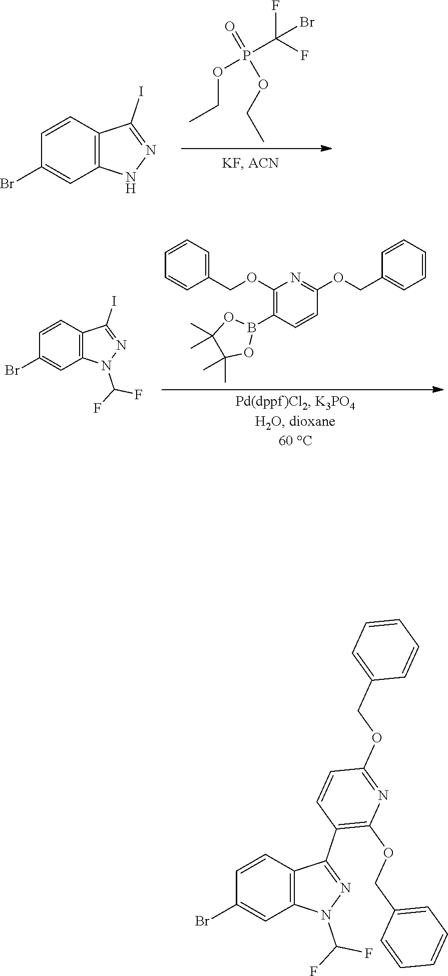 [SMILES: Brc1ccc2c(I)n[nH]c2c1 ; CCOP(=O)(OCC)C(F)(F)Br ; FC(F)n1nc(I)c2ccc(Br)cc21 ; CC1(C)OB(c2ccc(OCc3ccccc3)nc2OCc2ccccc2)OC1(C)C ; FC(F)n1nc(-c2ccc(OCc3ccccc3)nc2OCc2ccccc2)c2ccc(Br)cc21]

### Step 1. Preparation of 6-bromo-1-(difluoromethyl)-3-iodo-1H-indazole

To a stirred solution of 6-bromo-3-iodo-1H-indazole (50. g, 150 mmol) in ACN (500 mL) was added KF (18 g, 310 mmol) at room temperature under nitrogen atmosphere. The reaction mixture was stirred for 30 min then diethyl (bromodifluoromethyl)phosphonate (41 g, 0.15 mol) was added dropwise. The reaction mixture was stirred at room temperature for 16 h then diluted with H₂O (100 mL), extracted with EtOAc (3×100 mL) and washed with brine (2×200 mL). The combined organic layer was dried over Na₂SO₄ and concentrated under reduced pressure to an orange gum. The residue was purified by column chromatography (silica gel; 0-1% EtOac: hexane) to provide 6-bromo-1-(difluoromethyl)-3-iodo-1H-indazole (20. g, 35% yield) as a white solid.

### Step 2. Preparation of 3-(2,6-bis(benzyloxy)pyridin-3-yl)-6-bromo-1-(difluoromethyl)-1H-indazole

A mixture of 6-bromo-1-(difluoromethyl)-3-iodo-1H-indazole (25.0 g, 59.7 mmol) and 2,6-bis(benzyloxy)-3-(4,4,5,5-tetramethyl-1,3,2-dioxaborolan-2-yl)pyridine (25 g, 0.60 mol) in dioxane (250 mL) and H₂O (30 mL) was combined with K₃PO₄ (20. g, 0.15 mol). The reaction mixture was degassed with nitrogen gas for 3 min, after which Pd(dppf)Cl₂ (4.4 g, 6.0 mmol) was added under a nitrogen gas. The reaction mixture was degassed again with nitrogen gas for 3 min and stirred at 60 °C for 12 h. The reaction mixture was diluted with H₂O (100 mL), extracted with EtOAc (3×100 mL), washed with brine (2×50 mL), dried over Na₂SO₄, and concentrated under reduced pressure to afford black gum. The crude product was purified by column chromatography (silica gel; 0-2% EtOAc: n-hexane) to provide 3-(2,6-bis(benzyloxy)pyridin-3-yl)-6-bromo-1-(difluoromethyl)-1H-indazole (17 g, 53% yield) as a white solid. LC/MS m/z [M+H]⁺=537.9.

### Preparation F46

### rac-(R)-3-(1-(difluoromethyl)-6-(1-(2-(methylamino)ethyl)piperidin-4-yl)-1H-indazol-3-yl)piperidine-2,6-dione dihydrochloride

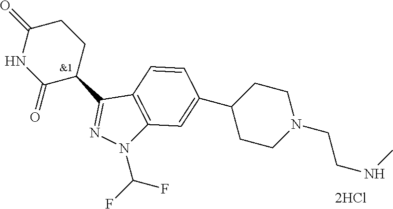 [SMILES: CNCCN1CCC(c2ccc3c([C@@H]4CCC(=O)NC4=O)nn(C(F)F)c3c2)CC1]

Made in the manner of Preparation F23 from Preparation F43. The acidic deprotection conditions were altered to use HCl instead of TFA. LC/MS m/z [M+H]⁺=420.2. ¹H NMR (400 MHz, (CD₃)₂SO) δ 11.02-10.98 (m, 1H), 9.41 (s, 2H), 8.44-7.90 (m, 1H), 7.84 (d, 1H), 7.66 (s, 1H), 7.25 (d, 1H), 4.49 (dd, 1H), 3.76-3.68 (m, 4H), 3.49-3.45 (m, 4H), 3.27-2.98 (m, 2H), 2.78-2.67 (m, 1H), 2.66-2.51 (m, 4H), 2.45-2.31 (m, 1H), 2.26-2.04 (m, 5H).

### Preparation F47

### 1-(4-(1-(2-(Methylamino)ethyl)piperidin-4-yl)benzyl)dihydropyrimidine-2,4(1H,3H)-dione

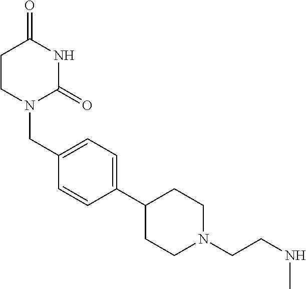 [SMILES: CNCCN1CCC(c2ccc(CN3CCC(=O)NC3=O)cc2)CC1]

Made in the manner of Preparation F6 from CAS: 2446914-05-0. LC/MS m/z [M+H]⁺=345.2.

### Preparation F48

### 1-(1-Methyl-6-(methyl(1-(2-(methylamino)ethyl)piperidin-4-yl)amino)-1H-indazol-3-yl)dihydropyrimidine-2,4(1H,3H)-dione

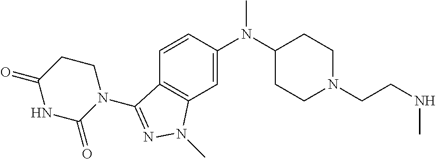 [SMILES: CNCCN1CCC(N(C)c2ccc3c(N4CCC(=O)NC4=O)nn(C)c3c2)CC1]

Made in the manner of Preparation F6 from CAS: 3085037-49-3. LC/MS m/z [M+H]⁺=414.3.

### Preparation F49

### 3-(2,4-Dioxotetrahydropyrimidin-1(2H)-yl)-7-fluoro-1-methyl-1H-indazole-6-carbaldehyde

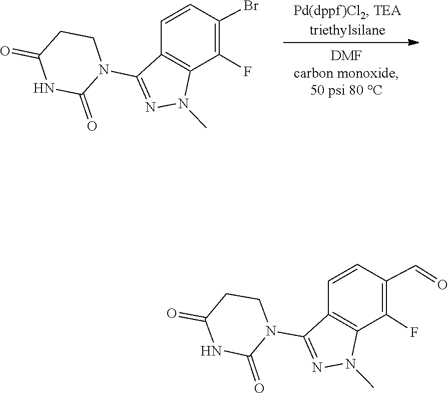 [SMILES: Cn1nc(N2CCC(=O)NC2=O)c2ccc(Br)c(F)c21 ; Cn1nc(N2CCC(=O)NC2=O)c2ccc(C=O)c(F)c21]

### Step 1. Preparation of 3-(2,4-dioxotetrahydropyrimidin-1(2H)-yl)-7-fluoro-1-methyl-1H-indazole-6-carbaldehyde

To a solution of 1-(6-bromo-7-fluoro-1-methyl-1H-indazol-3-yl)dihydropyrimidine-2,4(1H,3H)-dione (CAS: 2760849-95-2; 0.500 g, 1.47 mmol) in DMF (2.5 mL) were added TEA (0.445 g, 4.40 mmol), triethylsilane (0.511 g, 4.40 mmol), and Pd(dppf)Cl₂ (107 mg, 0.147 mmol) at room temperature. The reaction mixture was degassed under vacuum and purged with argon three times, then stirred under carbon monoxide (50 psi) at 80 °C for 20 h. The reaction mixture was cooled to 0 °C before water (60 mL) was added dropwise. The resulting precipitate was filtered and concentrated in vacuo. The residue was triturated with ACN (20 mL) then filtered. The filter cake was collected and dried by high vacuum to provide 3-(2,4-dioxotetrahydropyrimidin-1(2H)-yl)-7-fluoro-1-methyl-1H-indazole-6-carbaldehyde (278 mg, 65% yield) as a pink solid. The pink solid was used directly into the next step without further purification. LC/MS m/z [M+H]⁺=291.1.

### Preparation F50

### 1-(7-Fluoro-1-methyl-6-((4-(2-(methylamino)ethyl)piperidin-1-yl)methyl)-1H-indazol-3-yl)dihydropyrimidine-2,4(1H,3H)-dione bis(2,2,2-trifluoroacetate)

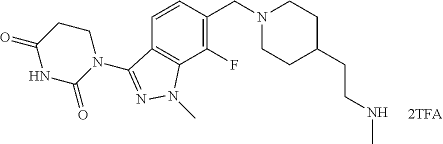 [SMILES: CNCCC1CCN(Cc2ccc3c(N4CCC(=O)NC4=O)nn(C)c3c2F)CC1]

Synthesized according to Preparation F3 with Preparation F49. LC/MS m/z [M+H]⁺=417.2. ¹H NMR (400 MHz, (CD₃)₂SO) δ 10.65 (s, 1H), 9.86 (s, 1H), 8.43 (s, 2H), 7.55 (d, 1H), 7.22 (dd, 1H), 4.61-4.41 (m, 2H), 4.15 (s, 3H), 3.93 (t, 2H), 3.27-3.17 (m, 1H), 3.11-2.83 (m, 4H), 2.77 (t, 2H), 2.56 (t, 3H), 2.46-2.41 (m, 1H), 1.91-1.62 (m, 2H), 1.61-1.28 (m, 5H).

### Preparation F51

### rac-(R)-3-(3-Methyl-5-(4-(2-(methylamino)ethyl)piperazin-1-yl)-1H-indazol-1-yl)piperidine-2,6-dione bis(2,2,2-trifluoroacetate)

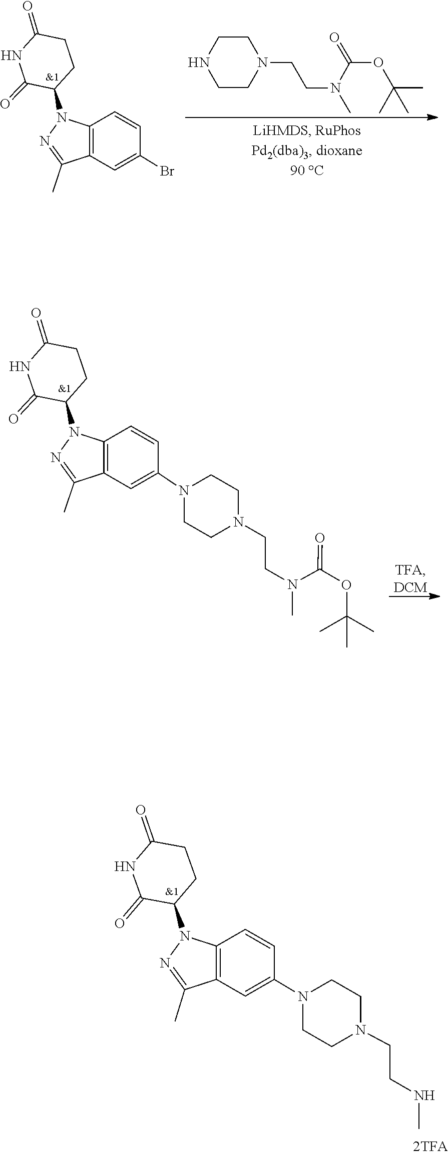 [SMILES: Cc1nn([C@@H]2CCC(=O)NC2=O)c2ccc(Br)cc12 ; CN(CCN1CCNCC1)C(=O)OC(C)(C)C ; Cc1nn([C@@H]2CCC(=O)NC2=O)c2ccc(N3CCN(CCN(C)C(=O)OC(C)(C)C)CC3)cc12 ; CNCCN1CCN(c2ccc3c(c2)c(C)nn3[C@@H]2CCC(=O)NC2=O)CC1]

### Step 1. Preparation of rac-tert-butyl (R)-(2-(4-(1-(2,6-dioxopiperidin-3-yl)-3-methyl-1H-indazol-5-yl)piperazin-1-yl)ethyl)(methyl)carbamate

The same reaction was conducted in two batches then combined for purification. To a stirred mixture of rac-(R)-3-(5-bromo-3-methyl-1H-indazol-1-yl)piperidine-2,6-dione (CAS: 2888607-41-6; 0.200 g, 0.621 mmol) in dioxane (5 mL) were added tert-butyl methyl(2-(piperazin-1-yl)ethyl)carbamate (CAS: 1540928-69-5; 181 mg, 0.745 mmol), RuPhos (29.0 mg, 0.0621 mmol), and Pd₂(dba)₃ (28.4 mg, 0.0310 mmol) to form the first batch. The reaction mixture was bubbled with nitrogen, followed by the addition of LiHMDS (519 mg, 3.10 mmol). The reaction mixture was stirred at 90 °C for 2 h.

A second batch of the same reaction occurred with rac-(R)-3-(5-bromo-3-methyl-1H-indazol-1-yl)piperidine-2,6-dione (CAS: 2888607-41-6; 0.100 g, 0.310 mmol). The two batches were combined then partitioned between water (20 mL) and EtOAc (3×20 mL). The combined organic layers were washed with water, dried over Na₂SO₄, and concentrated in vacuo. The residue was purified by column chromatography (silica gel; 0-10% MeOH: DCM) to provide rac-tert-butyl (R)-(2-(4-(1-(2,6-dioxopiperidin-3-yl)-3-methyl-1H-indazol-5-yl)piperazin-1-yl)ethyl)(methyl)carbamate (282 mg, 62.5% yield) as a yellow glass. LC/MS m/z [M+H]⁺=485.4.

### Step 2. Preparation of rac-(R)-3-(3-methyl-5-(4-(2-(methylamino)ethyl)piperazin-1-yl)-1H-indazol-1-yl)piperidine-2,6-dione bis(2,2,2-trifluoroacetate)

To a solution of rac-tert-butyl (R)-(2-(4-(1-(2,6-dioxopiperidin-3-yl)-3-methyl-1H-indazol-5-yl)piperazin-1-yl)ethyl)(methyl)carbamate (0.282 g, 0.582 mmol) in DCM (4 mL) was added TFA (4 mL). The reaction mixture was stirred at room temperature for 3 h. The mixture was concentrated under reduced pressure. The residue was diluted with water (20 mL) then extracted with EtOAc (20 mL×2). The aqueous phase was lyophilized to give rac-(R)-3-(3-methyl-5-(4-(2-(methylamino)ethyl)piperazin-1-yl)-1H-indazol-1-yl)piperidine-2,6-dione bis(2,2,2-trifluoroacetate) (0.201 g, crude) as a brown solid. The solid was used directly in the next step without further purification. LC/MS m/z [M+H]⁺=385.2. ¹H NMR (400 MHz, (CD₃)₂SO) δ 11.04 (s, 1H), 8.85 (s, 2H), 7.49 (d, 1H), 7.44-7.26 (m, 2H), 7.24-7.15 (m, 1H), 5.69 (dd, 1H), 2.91-2.78 (m, 2H), 2.77-2.67 (m, 2H), 2.67-2.63 (m, 5H), 2.47-2.42 (m, 4H), 2.26-2.17 (m, 1H)(product peaks only).

### Preparation F52

### rac-(R)-3-(3-Fluoro-4-(1-(2-(methylamino)ethyl)piperidin-4-yl)phenyl)piperidine-2,6-dione bis(2,2,2-trifluoroacetate)

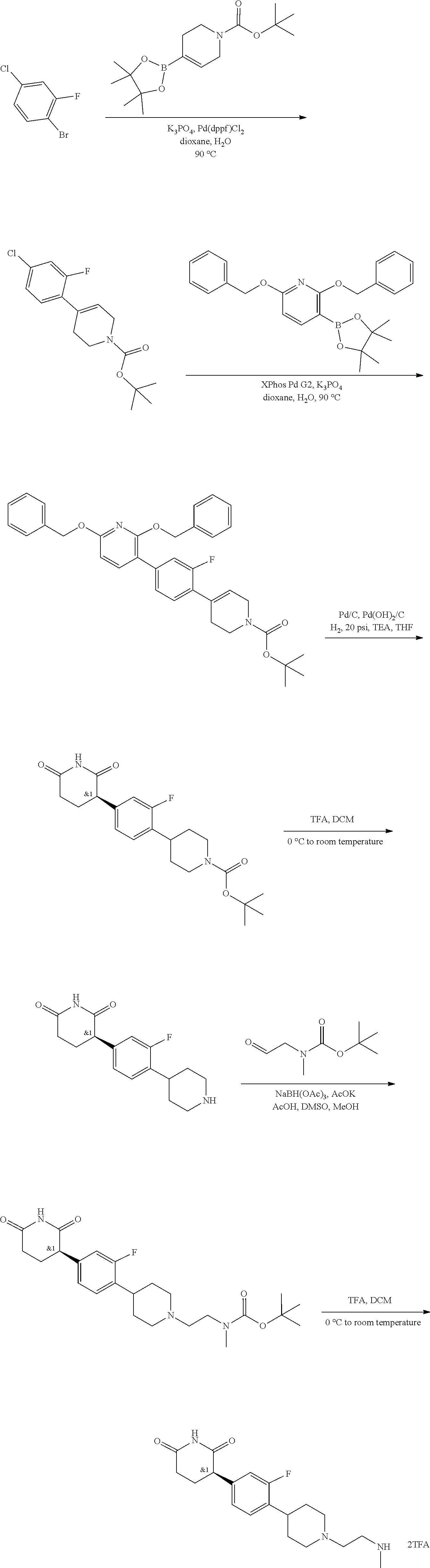

### Step 1. Preparation of tert-butyl 4-(4-chloro-2-fluorophenyl)-3,6-dihydropyridine-1(2H)-carboxylate

The same reaction was conducted in two batches then combined for purification. To a reaction mixture of 1-bromo-4-chloro-2-fluorobenzene (CAS: 1996-29-8; 2.00 g, 9.55 mmol), tert-butyl 4-(4,4,5,5-tetramethyl-1,3,2-dioxaborolan-2-yl)-3,6-dihydropyridine-1(2H)-carboxylate (2.95 g, 9.55 mmol) in dioxane (40 mL) and H₂O (8 mL) was added K3PO₄ (5.07 g, 23.9 mmol) to form the first batch. The reaction mixture of the first batch was degassed with nitrogen gas for 3 min then Pd(dppf)Cl₂ (0.699 g, 0.955 mmol) was added under a nitrogen atmosphere. The reaction mixture of the first batch was degassed with nitrogen gas for 3 min then stirred at 90 °C for 2 h.

A second batch of the same reaction was conducted with 1-bromo-4-chloro-2-fluorobenzene (CAS: 1996-29-8; 6.00 g, 28.6 mmol). The batches were combined then poured into H₂O (100 mL) and extracted with EtOAc (100 mL×3). The combined organic layer was purified by column chromatography (silica gel; 0-30% EtOAc: petroleum ether) to give tert-butyl 4-(4-chloro-2-fluorophenyl)-3,6-dihydropyridine-1(2H)-carboxylate (11.9 g, 99.9% yield) as a yellow gum. LC/MS m/z [M-tert-butyl+H]⁺=256.0. ¹H NMR (400 MHz, (CD₃)₂SO) δ 7.46-7.31 (m, 2H), 7.28 (dd, 1H), 6.01 (s, 1H), 4.01-3.96 (m, 2H), 3.51 (t, 2H), 2.42-2.38 (m, 2H), 1.42 (s, 9H).

### Step 2. Preparation of tert-butyl 4-(4-(2,6-bis(benzyloxy)pyridin-3-yl)-2-fluorophenyl)-3,6-dihydropyridine-1(2H)-carboxylate

To a solution of tert-butyl 4-(4-chloro-2-fluorophenyl)-3,6-dihydropyridine-1(2H)-carboxylate (0.500 g, 1.60 mmol), 2,6-bis(benzyloxy)-3-(4,4,5,5-tetramethyl-1,3,2-dioxaborolan-2-yl)pyridine (CAS: 2152673-80-6; 0.669 g, 1.60 mmol) in dioxane (12 mL) and H₂O (3 mL) was added K3PO₄ (1.02 g, 4.81 mmol). The mixture was degassed with nitrogen for 3 min then XPhos Pd G2 (126 mg, 0.160 mmol) was added under a nitrogen atmosphere then the reaction mixture was degassed with nitrogen gas for 3 min. The reaction mixture was stirred at 90 °C for 2 h. The reaction mixture was diluted with H₂O (20 mL), then extracted with EtOAc (3×30 mL). The combined organic layers were washed with brine (2×20 mL), dried over Na₂SO₄, and concentrated under reduced pressure to give a black gum. The residue was purified by column chromatography (silica gel; 0-15% EtOAc: hexane) to afford tert-butyl 4-(4-(2,6-bis(benzyloxy)pyridin-3-yl)-2-fluorophenyl)-3,6-dihydropyridine-1(2H)-carboxylate (0.670 g, 73.7% yield) as a yellow gum. LC/MS m/z [M+H]⁺=567.2. ¹H NMR (400 MHz, (CD₃)₂SO) δ 7.83 (d, 1H), 7.47-7.26 (m, 13H), 6.57 (d, 1H), 6.03 (s, 1H), 5.43 (s, 2H), 5.38 (s, 2H), 4.02-3.97 (m, 2H), 3.52 (t, 2H), 2.46-2.42 (m, 2H), 1.43 (s, 9H).

### Step 3. Preparation of rac-tert-butyl (R)-4-(4-(2,6-dioxopiperidin-3-yl)-2-fluorophenyl)piperidine-1-carboxylate

To a solution of tert-butyl 4-(4-(2,6-bis(benzyloxy)pyridin-3-yl)-2-fluorophenyl)-3,6-dihydropyridine-1(2H)-carboxylate (0.670 g, 1.18 mmol) in THF (9 mL) was added TEA (0.120 g, 1.18 mmol), followed by the addition of 10% Pd/C (126 mg, 0.118 mmol) and 10% Pd(OH)₂/C (0.166 g, 0.118 mmol). The reaction mixture was stirred under hydrogen gas at room temperature and 20 psi for 16 h. The mixture was then filtered through a pad of Celite, and the filtrate was concentrated under reduced pressure to give rac-tert-butyl (R)-4-(4-(2,6-dioxopiperidin-3-yl)-2-fluorophenyl)piperidine-1-carboxylate (0.390 g, crude) as a white solid. The gum was used directly into the next step without further purification. LC/MS m/z [M-tert-butyl+H]⁺=335.1. ¹H NMR (400 MHz, (CD₃)₂SO) δ 10.84 (s, 1H), 7.28 (t, 1H), 7.09-6.98 (m, 2H), 4.11-4.01 (m, 2H), 3.86 (dd, 1H), 3.65-3.56 (m, 1H), 3.01-2.89 (m, 1H), 2.73-2.59 (m, 1H), 2.54-2.51 (m, 1H), 2.32-2.14 (m, 1H), 2.07-1.95 (m, 1H), 1.80-1.67 (m, 3H), 1.61-1.44 (m, 2H), 1.41 (s, 9H).

### Step 4. Preparation of rac-(R)-3-(3-fluoro-4-(piperidin-4-yl)phenyl)piperidine-2,6-dione trifluoroacetate

To a stirred solution of rac-tert-butyl (R)-4-(4-(2,6-dioxopiperidin-3-yl)-2-fluorophenyl)piperidine-1-carboxylate (0.390 g, 0.999 mmol) in DCM (3 mL) was added TFA (2 mL) at 0 °C. The reaction mixture was warmed to room temperature and stirred for 2 h. The mixture was concentrated under reduced pressure to afford a yellow gum. The gum was dissolved in ACN (2 mL) and H₂O (10 mL) then lyophilized overnight to give rac-(R)-3-(3-fluoro-4-(piperidin-4-yl)phenyl)piperidine-2,6-dione trifluoroacetate (0.404 g, crude) as a yellow gum. The gum was used directly into the next step without further purification. LC/MS m/z [M+H]⁺=291.1.

### Step 5. Preparation of rac-tert-butyl (R)-(2-(4-(4-(2,6-dioxopiperidin-3-yl)-2-fluorophenyl)piperidin-1-yl)ethyl)(methyl)carbamate

To a solution of rac-(R)-3-(3-fluoro-4-(piperidin-4-yl)phenyl)piperidine-2,6-dione trifluoroacetate (0.404 g, 0.999 mmol) and tert-butyl methyl(2-oxoethyl)carbamate (CAS: 123387-72-4; 182 mg, 1.05 mmol) in DMSO (6.0 mL) and MeOH (0.6 mL) was added AcOK (343 mg, 3.50 mmol), AcOH (0.120 g, 2.00 mmol) then NaBH(OAc)₃ (0.424 g, 2.00 mmol). The reaction mixture was degassed with nitrogen gas for 2 min then stirred at room temperature for 16 h then poured into water (10 mL) and extracted with DCM (10 mL×3). The combined organic phase was washed with brine (10 mL×2), dried with Na₂SO₄ and concentrated in vacuo to provide rac-tert-butyl (R)-(2-(4-(4-(2,6-dioxopiperidin-3-yl)-2-fluorophenyl)piperidin-1-yl)ethyl)(methyl)carbamate (0.417 g, crude) as a white solid. The solid was used directly in the next step without further purification. (LC/MS) m/z (M+H)⁺=448.3.

### Step 6. Preparation of rac-(R)-3-(3-fluoro-4-(1-(2-(methylamino)ethyl)piperidin-4-yl)phenyl)piperidine-2,6-dione bis(2,2,2-trifluoroacetate)

At 0 °C, to a solution of rac-tert-butyl (R)-(2-(4-(4-(2,6-dioxopiperidin-3-yl)-2-fluorophenyl)piperidin-1-yl)ethyl)(methyl)carbamate (0.417 g, 0.932 mmol) in DCM (3.0 mL) was added TFA (2.0 mL) then stirred at room temperature for 2 h. The mixture was concentrated in vacuo to give a yellow gum. The residue was dissolved in ACN (2 mL) then diluted with H₂O (10 mL) and lyophilized overnight to give rac-(R)-3-(3-fluoro-4-(1-(2-(methylamino)ethyl)piperidin-4-yl)phenyl)piperidine-2,6-dione bis(2,2,2-trifluoroacetate) (0.670 g, crude) as a yellow gum. The gum was used directly in the next step without further purification. (LC/MS) m/z (M+H)⁺=348.3. ¹H NMR (400 MHz, (CD₃)₂SO) δ 10.86 (s, 1H), 9.96 (s, 1H), 8.83 (s, 2H), 7.25 (t, 1H), 7.14-7.04 (m, 2H), 3.89 (dd, 1H), 3.73-3.56 (m, 2H), 3.25-3.04 (m, 3H), 2.67-2.63 (m, 3H), 2.54 (s, 6H), 2.41-2.16 (m, 1H), 2.04-1.96 (m, 5H).

### Preparation F53

### 1-(6-([4,4′-Bipiperidin]-1-ylmethyl)-1-methyl-1H-indazol-3-yl)dihydropyrimidine-2,4(1H,3H)-dione dihydrochloride

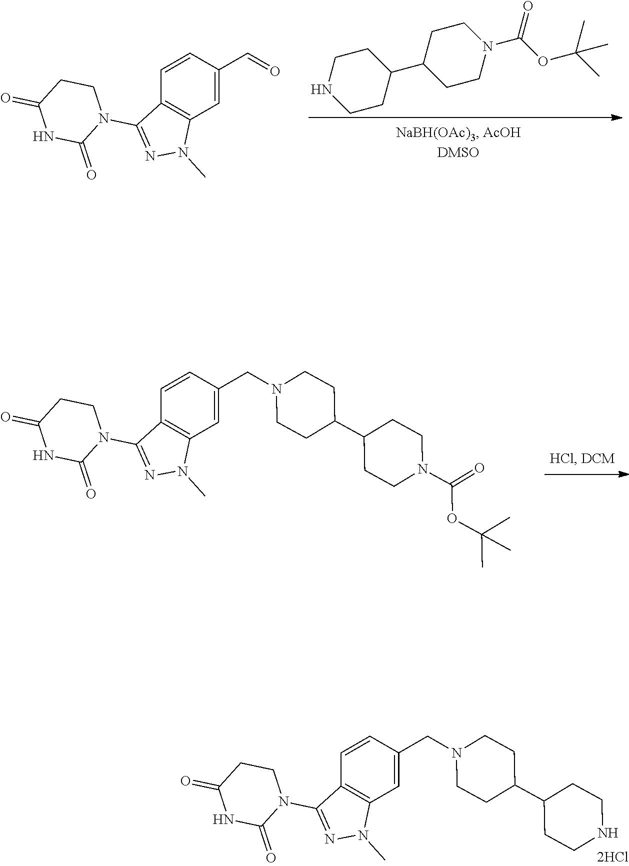 [SMILES: Cn1nc(N2CCC(=O)NC2=O)c2ccc(C=O)cc21 ; CC(C)(C)OC(=O)N1CCC(C2CCNCC2)CC1 ; Cn1nc(N2CCC(=O)NC2=O)c2ccc(CN3CCC(C4CCN(C(=O)OC(C)(C)C)CC4)CC3)cc21 ; Cn1nc(N2CCC(=O)NC2=O)c2ccc(CN3CCC(C4CCNCC4)CC3)cc21]

### Step 1. Preparation of tert-butyl 1′-((3-(2,4-dioxotetrahydropyrimidin-1(2H)-yl)-1-methyl-1H-indazol-6-yl)methyl)-[4,4′-bipiperidine]-1-carboxylate

To a solution of 3-(2,4-dioxotetrahydropyrimidin-1(2H)-yl)-1-methyl-1H-indazole-6-carbaldehyde (CAS: 2925088-72-6; 0.2 g, 0.7 mmol), tert-butyl [4,4′-bipiperidine]-1-carboxylate (0.2 g, 0.7 mmol) in DMSO (4 mL) was added AcOH (0.04 g, 0.04 mL, 0.7 mmol) followed by NaBH(OAc)₃ (0.3 g, 1 mmol). The reaction was stirred overnight. The slurry was diluted with H₂O (10 mL) then extracted with (1:10)(MeOH: DCM)(10 mL×3). The combined organic layers were washed with brine (5 mL) then dried with MgSO₄. The suspension was filtered over Celite then the filtrate was concentrated under reduced pressure to give tert-butyl 1′-((3-(2,4-dioxotetrahydropyrimidin-1(2H)-yl)-1-methyl-1H-indazol-6-yl)methyl)-[4,4′-bipiperidine]-1-carboxylate (0.4 g, crude) as a yellow gum. The gum was used directly into the next step without further purification. (LC/MS) m/z (M+H)⁺=525.5.

### Step 2. Preparation of 1-(6-([4,4′-bipiperidin]-1-ylmethyl)-1-methyl-1H-indazol-3-yl)dihydropyrimidine-2,4(1H,3H)-dione dihydrochloride

To a solution of tert-butyl 1′-((3-(2,4-dioxotetrahydropyrimidin-1(2H)-yl)-1-methyl-1H-indazol-6-yl)methyl)-[4,4′-bipiperidine]-1-carboxylate (0.3 g, 0.6 mmol) in DCM (3 mL) under nitrogen atmosphere was added 4M HCl (0.3 g, 2 mL, 9 mmol) slowly. The reaction mixture was stirred at room temperature for 3 h then concentrated by reduced pressure. The residue triturated with heptane (2×5 mL). The crude material was dried under high vacuum for 30 min to afford 1-(6-([4,4′-bipiperidin]-1-ylmethyl)-1-methyl-1H-indazol-3-yl)dihydropyrimidine-2,4(1H,3H)-dione dihydrochloride (0.3 g, crude) a light-yellow solid. The solid was used directly into the next step without further purification.

### Preparation F54

### 1-(1-Methyl-6-(piperidin-4-yloxy)-1H-indazol-3-yl)dihydropyrimidine-2,4(1H,3H)-dione hydrochloride

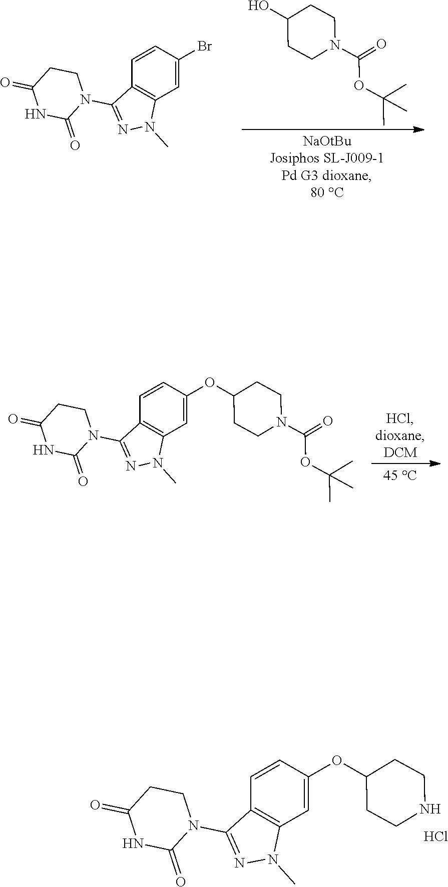 [SMILES: Cn1nc(N2CCC(=O)NC2=O)c2ccc(Br)cc21 ; CC(C)(C)OC(=O)N1CCC(O)CC1 ; Cn1nc(N2CCC(=O)NC2=O)c2ccc(OC3CCN(C(=O)OC(C)(C)C)CC3)cc21 ; Cn1nc(N2CCC(=O)NC2=O)c2ccc(OC3CCNCC3)cc21 ; Cl]

### Step 1. Preparation of tert-butyl 4-((3-(2,4-dioxotetrahydropyrimidin-1(2H)-yl)-1-methyl-1H-indazol-6-yl)oxy)piperidine-1-carboxylate

The same reaction was conducted in two batches then combined for purification. To a vial were added 1-(6-bromo-1-methyl-1H-indazol-3-yl)dihydropyrimidine-2,4(1H,3H)-dione (CAS: 2446913-88-6; 0.050 g, 0.15 mmol), tert-butyl 4-hydroxypiperidine-1-carboxylate (CAS: 109384-19-2; 37 mg, 0.19 mmol), NaOtBu (59 mg, 0.62 mmol), and JosiPhos SL-J009-1 Pd G3 (22 mg, 0.023 mmol) to form the first batch. The vial of the first batch was purged with nitrogen gas then dioxane (1 mL) was added. The reaction mixture of the first batch was stirred at 80 °C for 1 h.

The second batch of the same reaction was conducted with 1-(6-bromo-1-methyl-1H-indazol-3-yl)dihydropyrimidine-2,4(1H,3H)-dione (CAS: 2446913-88-6; 1.0 g, 3.1 mmol) with the same conditions except the reaction solvent was toluene (8 mL) and dioxane (2 mL). The two batches were combined then concentrated under reduced pressure. The residue was dissolved in DCM then purified by column chromatography (silica gel; 0-100% EtOAc: heptane) to give tert-butyl 4-((3-(2,4-dioxotetrahydropyrimidin-1(2H)-yl)-1-methyl-1H-indazol-6-yl)oxy)piperidine-1-carboxylate (0.20 g, 14% yield) as an orange oil. (LC/MS) m/z (M+H)⁺=444.3.

### Step 2. Preparation of 1-(1-methyl-6-(piperidin-4-yloxy)-1H-indazol-3-yl)dihydropyrimidine-2,4(1H,3H)-dione hydrochloride

To a solution of tert-butyl 4-((3-(2,4-dioxotetrahydropyrimidin-1(2H)-yl)-1-methyl-1H-indazol-6-yl)oxy)piperidine-1-carboxylate (0.200 g, 0.451 mmol) in DCM (2 mL) was added 4M HCl in dioxane (65.8 mg, 0.4 mL, 1.80 mmol). The reaction mixture was stirred at 45 °C for −2 h then concentrated under reduced pressure to afford 1-(1-methyl-6-(piperidin-4-yloxy)-1H-indazol-3-yl)dihydropyrimidine-2,4(1H,3H)-dione hydrochloride (0.171 g, crude) as a red residue. The red residue was used directly in the next step without further purification. (LC/MS) m/z (M+H)⁺=344.2.

### Preparation F55

### 1-(1-Methyl-6-((1-(2-(methylamino)ethyl)piperidin-4-yl)oxy)-1H-indazol-3-yl)dihydropyrimidine-2,4(1H,3H)-dione

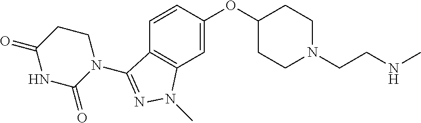 [SMILES: CNCCN1CCC(Oc2ccc3c(N4CCC(=O)NC4=O)nn(C)c3c2)CC1]

Synthesized according to Preparation F6 with Preparation F54. The acidic deprotection was altered to utilize 4M HCl in dioxane. LC/MS m/z [M+H]⁺=401.2.

### Preparation F56

### 2,2,2-Trifluoro-N-(2-(4-fluoropiperidin-4-yl)ethyl)-N-methylacetamide hydrochloride

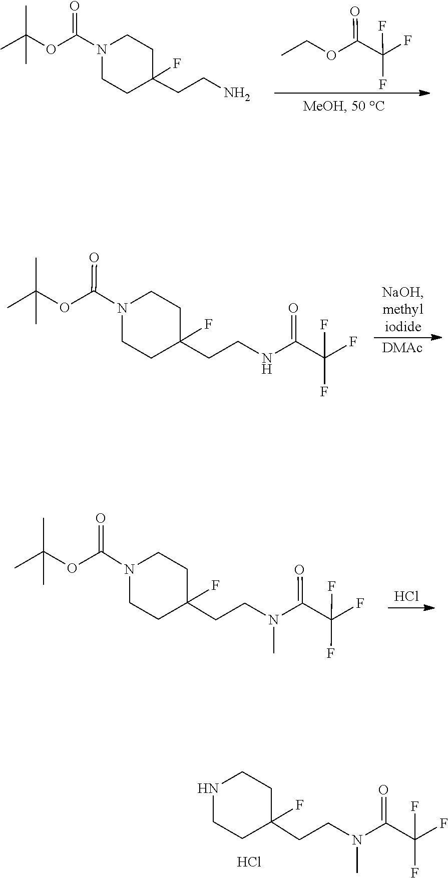

### Step 1. Preparation of tert-butyl 4-fluoro-4-(2-(2,2,2-trifluoroacetamido)ethyl)piperidine-1-carboxylate

To a vial were added tert-butyl 4-(2-aminoethyl)-4-fluoropiperidine-1-carboxylate (1.00 g, 4.06 mmol) and ethyl 2,2,2-trifluoroacetate (1.15 g, 1.0 mL, 8.12 mmol) in MeOH (10.0 mL). The reaction mixture was stirred at 50 °C for 10 h, then cooled to room temperature and stirred at room temperature overnight. The solvent was distillation to dryness to afford tert-butyl 4-fluoro-4-(2-(2,2,2-trifluoroacetamido)ethyl)piperidine-1-carboxylate (1.39 g, crude) as a clear gum. The gum was used directly in the next step without further purification. ¹H NMR (400 MHz, (CD₃)₂SO) δ 9.44 (t, 1H), 4.09 (q, 1H), 3.75 (d, 2H), 3.36-3.26 (m, 1H), 3.17 (d, 2H), 1.96-1.71 (m, 4H), 1.67-1.45 (m, 2H), 1.39 (s, 9H).

### Step 2. Preparation of tert-butyl 4-fluoro-4-(2-(2,2,2-trifluoro-N-methylacetamido)ethyl)piperidine-1-carboxylate

To a vial containing tert-butyl 4-fluoro-4-(2-(2,2,2-trifluoroacetamido)ethyl)piperidine-1-carboxylate (1.34 g, 3.91 mmol) was added DMAc (13.4 mL), followed by methyl iodide (833 mg, 0.37 mL, 5.87 mmol) and NaOH (313 mg, 7.83 mmol) sequentially. The reaction mixture was stirred at room temperature for 3 h. Upon completion, the reaction mixture was diluted with brine then extracted with MTBE (3×). The combined organic layers were distilled to dryness to afford tert-butyl 4-fluoro-4-(2-(2,2,2-trifluoro-N-methylacetamido)ethyl)piperidine-1-carboxylate (1.36 g, crude) as a gum. The gum was used directly in the next step without further purification.

### Step 3. Preparation of 2,2,2-trifluoro-N-(2-(4-fluoropiperidin-4-yl)ethyl)-N-methylacetamide hydrochloride

To a vial was added tert-butyl 4-fluoro-4-(2-(2,2,2-trifluoro-N-methylacetamido)ethyl)piperidine-1-carboxylate (1.39 g, 3.90 mmol), followed by 2M HCl (1.42 g, 19.5 mL, 39.0 mmol). The reaction mixture was held at room temperature for 19 h, during which time a light slurry formed. The mixture was distilled to dryness, to afford 2,2,2-trifluoro-N-(2-(4-fluoropiperidin-4-yl)ethyl)-N-methylacetamide hydrochloride (1.14 g, crude) as a yellow wax. The wax was used directly into the next step without further purification.

### Preparation F57

### 1-(6-((4-Fluoro-4-(2-(methylamino)ethyl)piperidin-1-yl)methyl)-1-methyl-1H-indazol-3-yl)dihydropyrimidine-2,4(1H,3H)-dione

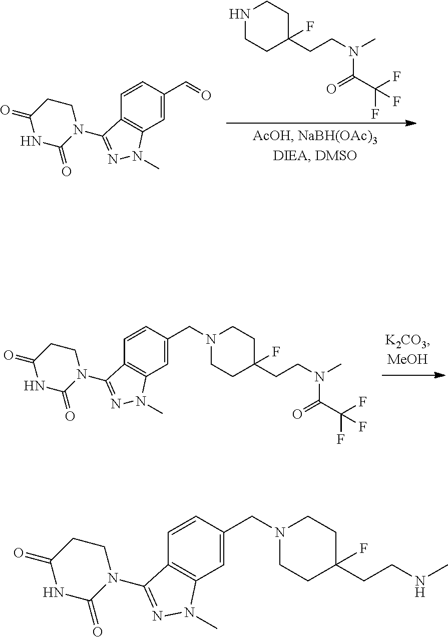 [SMILES: Cn1nc(N2CCC(=O)NC2=O)c2ccc(C=O)cc21 ; CN(CCC1(F)CCNCC1)C(=O)C(F)(F)F ; CN(CCC1(F)CCN(Cc2ccc3c(N4CCC(=O)NC4=O)nn(C)c3c2)CC1)C(=O)C(F)(F)F ; CNCCC1(F)CCN(Cc2ccc3c(N4CCC(=O)NC4=O)nn(C)c3c2)CC1]

### Step 1. Preparation of N-(2-(1-((3-(2,4-dioxotetrahydropyrimidin-1(2H)-yl)-1-methyl-1H-indazol-6-yl)methyl)-4-fluoropiperidin-4-yl)ethyl)-2,2,2-trifluoro-N-methylacetamide

To a vial were sequentially added 2,2,2-trifluoro-N-(2-(4-fluoropiperidin-4-yl)ethyl)-N-methylacetamide hydrochloride (0.970 g, 3.31 mmol), DMSO (20.70 mL), and DIEA (857 mg, 1.15 mL, 6.63 mmol). The mixture was stirred for 5 min, resulting in a slurry. To the reaction mixture was added AcOH (796 mg, 0.76 mL, 13.3 mmol) then 3-(2,4-dioxotetrahydropyrimidin-1(2H)-yl)-1-methyl-1H-indazole-6-carbaldehyde (902 mg, 3.31 mmol). To the mixture was added NaBH(OAc)₃ (2.81 g, 13.3 mmol) in one portion then the reaction was stirred at room temperature for 18 h. The reaction mixture was diluted with EtOAc and H₂O. The organic layer was separated. The aqueous layer was extracted with EtOAc (2×). The combined organic layers were purified by column chromatography (silica gel; 0-100% EtOAc: heptane) to provide N-(2-(1-((3-(2,4-dioxotetrahydropyrimidin-1(2H)-yl)-1-methyl-1H-indazol-6-yl)methyl)-4-fluoropiperidin-4-yl)ethyl)-2,2,2-trifluoro-N-methylacetamide (0.600 g, 35.3% yield) as a yellow oil. LC/MS m/z [M+H]⁺=513.5.

### Step 2. Preparation of 1-(6-((4-fluoro-4-(2-(methylamino)ethyl)piperidin-1-yl)methyl)-1-methyl-1H-indazol-3-yl)dihydropyrimidine-2,4(1H,3H)-dione

In a round bottom flask, a reaction mixture of N-(2-(1-((3-(2,4-dioxotetrahydropyrimidin-1(2H)-yl)-1-methyl-1H-indazol-6-yl)methyl)-4-fluoropiperidin-4-yl)ethyl)-2,2,2-trifluoro-N-methylacetamide (0.600 g, 1.17 mmol) in MeOH (13 mL) was added K₂CO₃ (0.809 g, 5.85 mmol). The suspension was stirred at room temperature for 3 h then filtered. The filtrate was distilled to dryness to give 1-(6-((4-fluoro-4-(2-(methylamino)ethyl)piperidin-1-yl)methyl)-1-methyl-1H-indazol-3-yl)dihydropyrimidine-2,4(1H,3H)-dione (0.488 g, crude) as an oil. The oil was used directly into the next step without further purification. (LC/MS) m/z (M+H)⁺=417.3.

### Preparation F58

### rac-(R)-3-((2-Chloro-4-(1-(2-(methylamino)ethyl)piperidin-4-yl)phenyl)amino)piperidine-2,6-dione bis(2,2,2-trifluoroacetate)

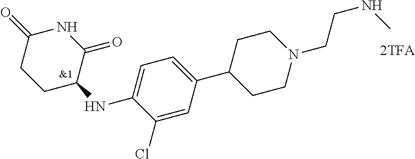 [SMILES: CNCCN1CCC(c2ccc(N[C@H]3CCC(=O)NC3=O)c(Cl)c2)CC1]

Synthesized according to Step 3 to 6 from Preparation F32 with CAS: 1386951-85-4. Step 5 utilized CAS: 123387-72-4. LC/MS m/z [M+H]⁺=379.2.

### Preparation F59

### 5-(Aminomethyl)-1,3-dihydrobenzo[c]thiophene 2,2-dioxide hydrochloride

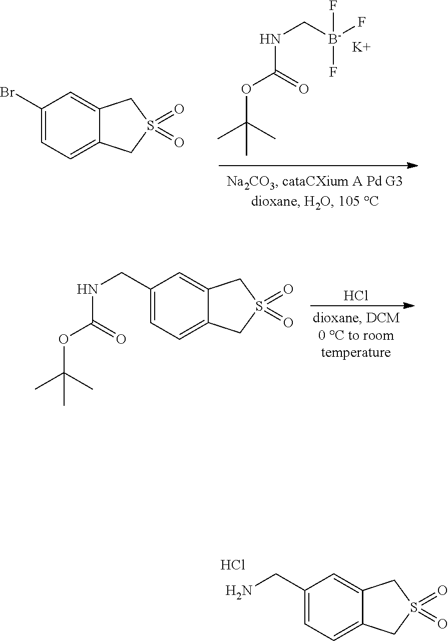

### Step 1. Preparation of tert-butyl ((2,2-dioxido-1,3-dihydrobenzo[c]thiophen-5-yl)methyl)carbamate

The same reaction was conducted in two batches then combined for purification. A mixture of 5-bromo-1,3-dihydrobenzo[c]thiophene 2,2-dioxide (CAS: 351005-12-4; 0.200 g, 0.809 mmol), potassium tert-butyl N-[(trifluoroboranuidyl)methyl]carbamate (CAS: 1314538-55-0; 0.230 g, 0.971 mmol), and Na₂CO₃ (214 mg, 2.02 mmol) in dioxane (6 mL) and H₂O (1.5 mL) was degassed with nitrogen gas (3×) before cataCXium A Pd G3 (118 mg, 0.162 mmol) was then added to form the first batch. The reaction mixture of the first batch was degassed with nitrogen gas (3×) before being stirred at 105 °C for 6 h.

A second batch of the same reaction was conducted with 5-bromo-1,3-dihydrobenzo[c]thiophene 2,2-dioxide (CAS: 351005-12-4; 50.0 mg, 0.202 mmol). The two batches were combined then filtered through Celite. The filter cake was washed with EtOAc (3×5 mL). The filtrate was poured into H₂O (15 mL) and extracted with EtOAc (2×15 mL). The combined organic layers were dried over Na₂SO₄, filtered, and concentrated by reduced pressure to afford a brown gum. The residue was purified by column chromatography (silica gel; 0-60% EtOAc: n-hexane) to provide tert-butyl ((2,2-dioxido-1,3-dihydrobenzo[c]thiophen-5-yl)methyl)carbamate (0.270 g, 89.7% yield) as a light brown solid. ¹H NMR (400 MHz, (CD₃)₂SO) δ 7.43 (t, 1H), 7.32 (d, 1H), 7.25-7.19 (m, 2H), 4.48 (s, 2H), 4.44 (s, 2H), 4.12 (d, 2H), 1.48-1.29 (m, 9H).

### Step 2. Preparation of 5-(aminomethyl)-1,3-dihydrobenzo[c]thiophene 2,2-dioxide hydrochloride

A mixture of tert-butyl ((2,2-dioxido-1,3-dihydrobenzo[c]thiophen-5-yl)methyl)carbamate (0.270 g, 0.908 mmol) in DCM (2.0 mL) was treated with hydrogen chloride in dioxane (5 mL) at 0-10 °C, and the reaction mixture was stirred at room temperature for 2 h. The solution was concentrated under reduced pressure to afford a light-yellow solid, which was lyophilized to give 5-(aminomethyl)-1,3-dihydrobenzo[c]thiophene 2,2-dioxide hydrochloride (0.220 g, crude) as a light yellow solid. The solid was used directly in the next step without further purification. LC/MS m/z [M+H]⁺=198.1. ¹H NMR (400 MHz, (CD₃)₂SO) δ 8.35 (s, 3H), 7.51-7.40 (m, 3H), 4.51 (d, 4H), 4.03 (q, 2H).

### Preparation F60

### (4-(1,3,4-Thiadiazol-2-yl)phenyl)methanamine hydrochloride

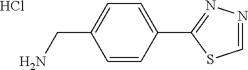 [SMILES: Cl ; NCc1ccc(-c2nncs2)cc1]

Synthesized according to Preparation F59 with CAS: 1388027-10-8. LC/MS m/z [M+H]⁺=192.1. ¹H NMR (400 MHz, (CD₃)₂SO) δ 9.67 (s, 1H), 8.64-8.46 (m, 2H), 8.11-8.03 (m, 2H), 7.71-7.65 (m, 2H), 4.15-4.07 (m, 2H).

### Preparation F61

### 1-(7-(Aminomethyl)-3,4-dihydroisoquinolin-2(1H)-yl)ethan-1-one hydrochloride

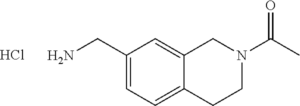

Synthesized according to Preparation F59 with CAS: 937591-28-1. LC/MS m/z [M+H]⁺=219.0.

### Preparation F62

### 4-(4-(Aminomethyl)phenyl)morpholin-3-one trifluoroacetate

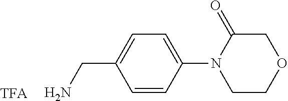 [SMILES: NCc1ccc(N2CCOCC2=O)cc1 ; "TFA"]

Synthesized according to Preparation F59 with CAS: 634905-12-7. The deprotection was altered to utilize TFA in DCM. ¹H NMR (400 MHz, (CD₃)₂SO) δ 8.21 (s, 3H), 7.53-7.43 (m, 4H), 4.21 (s, 2H), 4.05 (q, 2H), 4.02-3.96 (m, 2H), 3.78-3.71 (m, 2H).

### Preparation F63

### 1-(4-(Aminomethyl)phenyl)-4-methyl-1,4-dihydro-5H-tetrazol-5-one hydrochloride

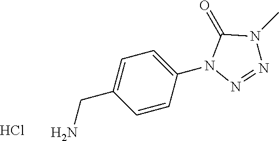

Synthesized according to Preparation F59 with CAS: 530080-39-8.

### Preparation F64

### 1-(1-Methyl-6-(4-((methylamino)methyl)piperidin-1-yl)-1H-indazol-3-yl)dihydropyrimidine-2,4(1H,3H)-dione hydrochloride

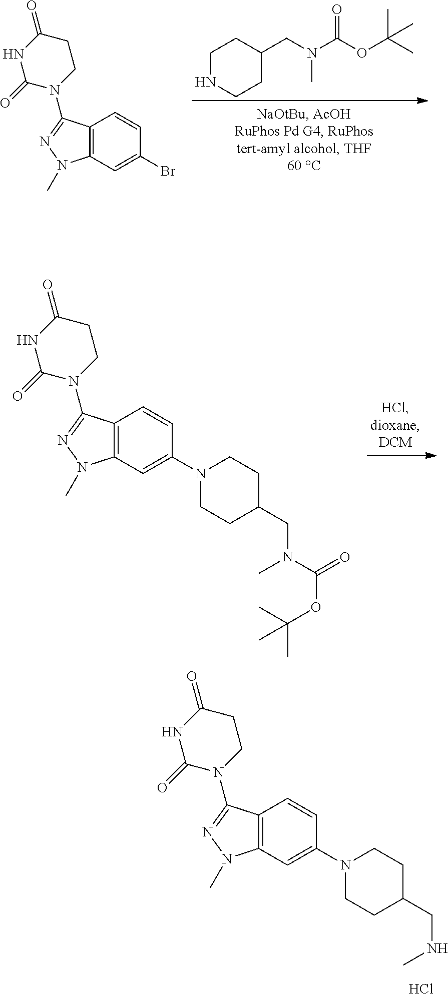 [SMILES: Cn1nc(N2CCC(=O)NC2=O)c2ccc(Br)cc21 ; CN(CC1CCNCC1)C(=O)OC(C)(C)C ; CN(CC1CCN(c2ccc3c(N4CCC(=O)NC4=O)nn(C)c3c2)CC1)C(=O)OC(C)(C)C ; CNCC1CCN(c2ccc3c(N4CCC(=O)NC4=O)nn(C)c3c2)CC1 ; Cl]

Step 1. Preparation of tert-butyl ((1-(3-(2,4-dioxotetrahydropyrimidin-1(2H)-yl)-1-methyl-1H-indazol-6-yl)piperidin-4-yl)methyl)(methyl)carbamate

A mixture of tert-butyl methyl(piperidin-4-ylmethyl)carbamate (CAS: 138022-04-5; 1.71 g, 7.34 mmol) and 1-(6-bromo-1-methyl-1H-indazol-3-yl)dihydropyrimidine-2,4(1H,3H)-dione (CAS: 2446913-88-6; 2.00 g, 6.12 mmol) in tert-amyl alcohol (15 mL) and THF (7 mL) was degassed under stirring with nitrogen gas for 10 min. To the reaction mixture were added RuPhos Pd G4 (208 mg, 0.245 mmol) and RuPhos (0.114 g, 0.245 mmol) followed by a solution of 2M NaOtBu in THF (1.53 g, 7.95 mL, 15.9 mmol). The reaction mixture was degassed for 2 min, then stirred at 60 °C for 2 h. After cooling, AcOH (918 mg, 0.87 mL, 15.3 mmol) was added then stirred for 5 min before EtOAc (5 mL), heptane (10 mL), and water (15 mL) were added. The reaction mixture was vigorously stirred for 15 min to form a biphasic solution. The organic phase was separated, dried over magnesium sulfate, and concentrated under reduced pressure to give a viscous yellow oil. The oil was dissolved in EtOAc (40 mL) and heptane (10 mL). To the solution was added water (15 mL) and 1 M KHSO₄ (2 mL), which resulted in precipitation. After vigorous stirring for 30 min, the mixture was filtered. The filter cake was washed with water (10 mL) and 20% heptane/EtOAc. The filter cake was dried on the filter for 1 h then dried further by vacuum oven at room temperature for ~72 h to give tert-butyl ((1-(3-(2,4-dioxotetrahydropyrimidin-1(2H)-yl)-1-methyl-1H-indazol-6-yl)piperidin-4-yl)methyl)(methyl)carbamate (1.89 g, 65.8% yield) as a solid.

The organic extract from the mother liquor was separated and concentrated. The residue was stirred with EtOAc (10 mL), heptane (5 mL), and water (1 mL) for 18 h. The resulting precipitate was filtered, washed with water and (2:1, EtOAc: heptane). The filter cake was dried on the filter then dried further in a vacuum oven at 50 °C for 3 h to provide tert-butyl ((1-(3-(2,4-dioxotetrahydropyrimidin-1(2H)-yl)-1-methyl-1H-indazol-6-yl)piperidin-4-yl)methyl)(methyl)carbamate (0.160 g, 6.57% yield) as a solid.

The two batches of product were combined to form tert-butyl ((1-(3-(2,4-dioxotetrahydropyrimidin-1(2H)-yl)-1-methyl-1H-indazol-6-yl)piperidin-4-yl)methyl)(methyl)carbamate (2.05 g, 71.2% yield) as a solid. (LC/MS) m/z (M+H)⁺=472.0. ¹H NMR (400 MHz, (CD₃)₂SO) δ 10.49 (s, 1H), 7.43 (d, 1H), 6.94-6.87 (m, 1H), 6.81 (s, 1H), 3.93-3.85 (m, 5H), 3.84-3.77 (m, 2H), 3.11 (d, 2H), 2.81 (s, 3H), 2.78-2.66 (m, 4H), 1.80-1.73 (m, 1H), 1.67 (d, 2H), 1.40 (s, 9H), 1.35-1.22 (m, 2H).

### Step 2. Preparation of 1-(1-methyl-6-(4-((methylamino)methyl)piperidin-1-yl)-1H-indazol-3-yl)dihydropyrimidine-2,4(1H,3H)-dione hydrochloride

To a solution of tert-butyl ((1-(3-(2,4-dioxotetrahydropyrimidin-1(2H)-yl)-1-methyl-1H-indazol-6-yl)piperidin-4-yl)methyl)(methyl)carbamate (825 mg, 1.75 mmol) in DCM (7 mL) was added a solution of 4M HCl in dioxane (0.220 g, 1.5 mL, 6.03 mmol) at room temperature in one portion. The reaction mixture was stirred at room temperature for 18 h, which caused solid to precipitate. The solid was filtered and the filter cake was washed with DCM and heptane. The filter cake was dried and collected to provide 1-(1-methyl-6-(4-((methylamino)methyl)piperidin-1-yl)-1H-indazol-3-yl)dihydropyrimidine-2,4(1H,3H)-dione hydrochloride (0.160 g, crude) as a solid. The solid was used directly in the next step without further purification. (LC/MS) m/z (M+H)⁺=371.4.

### Preparation F65

### 1-(1-Methyl-7-(4-((methylamino)methyl)piperidin-1-yl)-1H-indazol-3-yl)dihydropyrimidine-2,4(1H,3H)-dione hydrochloride

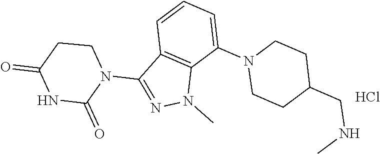 [SMILES: CNCC1CCN(c2cccc3c(N4CCC(=O)NC4=O)nn(C)c23)CC1 ; Cl]

Synthesized according to Preparation F64 with CAS: 2810876-98-1. (LC/MS) m/z (M+H)+=371.2. ¹H NMR (400 MHz, (CD₃)₂SO) δ 10.54 (s, 1H), 8.63-8.59 (m, 2H), 7.30 (dd, 1H), 7.09-6.97 (m, 2H), 5.76 (s, 2H), 4.24 (s, 3H), 3.88 (t, 2H), 3.57 (s, 1H), 3.32-3.26 (m, 1H), 2.93-2.89 (m, 2H), 2.78-2.64 (m, 3H), 2.59 (t, 3H), 1.90-1.85 (m, 2H), 1.53-1.46 (m, 2H).

### Preparation F66

### rac-(R)-1-(1-Methyl-6-((2-((methylamino)methyl)morpholino)methyl)-1H-indazol-3-yl)dihydropyrimidine-2,4(1H,3H)-dione dihydrochloriide

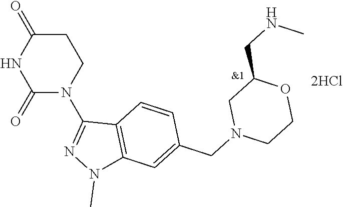 [SMILES: CNC[C@@H]1CN(Cc2ccc3c(N4CCC(=O)NC4=O)nn(C)c3c2)CCO1]

Synthesized according to Preparation F53 with CAS: 2925088-72-6. (LC/MS) m/z (M+H)⁺=387.4.

### Preparation F67

### 1-(6-((4-(Azetidin-3-yl)piperidin-1-yl)methyl)-1-methyl-1H-indazol-3-yl)dihydropyrimidine-2,4(1H,3H)-dione

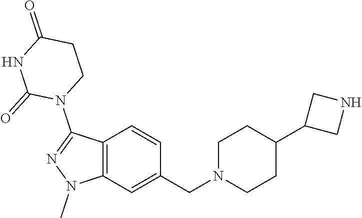 [SMILES: Cn1nc(N2CCC(=O)NC2=O)c2ccc(CN3CCC(C4CNC4)CC3)cc21]

Synthesized according to Preparation F53 with CAS: 2925088-72-6. (LC/MS) m/z (M+H)⁺=397.3.

### Preparation F68 and F69

### rel-5-(tert-Butyl) 3-ethyl (R)-1-ethyl-7-(trifluoromethyl)-1,4,6,7-tetrahydro-5H-pyrazolo[4,3-c]pyridine-3,5-dicarboxylate and rel-5-(tert-Butyl) 3-ethyl (S)-1-ethyl-7-(trifluoromethyl)-1,4,6,7-tetrahydro-5H-pyrazolo[4,3-c]pyridine-3,5-dicarboxylate

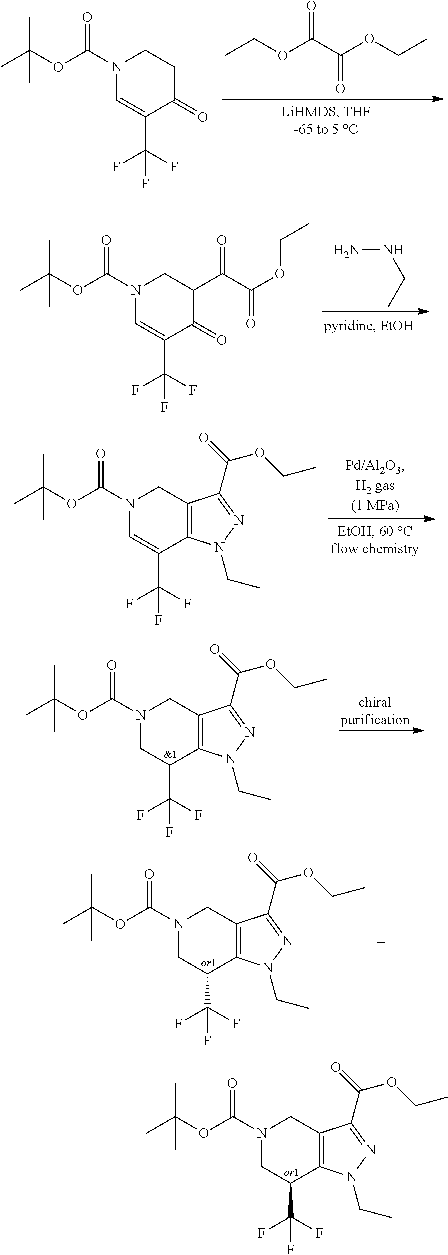

### Step 1. Preparation of tert-butyl 3-(2-ethoxy-2-oxoacetyl)-4-oxo-5-(trifluoromethyl)-3,4-dihydropyridine-1(2H)-carboxylate

In a dry 1000 mL two-necked round-bottom flask equipped with a magnetic stir bar, was added 1 M LiHMDS (14.6 g, 87.1 mL, 87.1 mmol) via addition funnel to THF (160 mL) under nitrogen gas. The solution was cooled to −65 °C using a dry ice/alcohol bath. A solution of tert-butyl 4-oxo-5-(trifluoromethyl)-3,4-dihydropyridine-1(2H)-carboxylate (CAS: 1667744-92-4; 21.0 g, 79.2 mmol) in THF (100 mL) was added dropwise via syringe over 20 min. The reaction was stirred at −65 °C for 1 h, then a solution of diethyl oxalate (12.1 g, 83.1 mmol) in THF (40 mL) was added dropwise over 5 min. The dry ice/alcohol bath was then replaced with an ice-water bath, and the reaction mixture was stirred at 0-5 °C for 1 h. At 0 °C, the reaction was carefully quenched by slow addition of 1 M HCl (120 mL) over 10 min while maintaining temperature below 10 °C. The mixture was diluted with water (100 mL) and extracted with EtOAc (3×200 mL). The combined organic layers were washed with brine (300 mL), dried over anhydrous Na₂SO₄, filtered, and concentrated to give tert-butyl 3-(2-ethoxy-2-oxoacetyl)-4-oxo-5-(trifluoromethyl)-3,4-dihydropyridine-1(2H)-carboxylate (28.9 g, crude) as an orange gum. The gum was used directly into the next step without further purification. (LC/MS) m/z (M+H)⁺=366.0.

### Step 2. Preparation of 5-(tert-butyl) 3-ethyl 1-ethyl-7-(trifluoromethyl)-1,4-dihydro-5H-pyrazolo[4,3-c]pyridine-3,5-dicarboxylate

To a reaction mixture of tert-butyl 3-(2-ethoxy-2-oxoacetyl)-4-oxo-5-(trifluoromethyl)-3,4-dihydropyridine-1(2H)-carboxylate (28.9 g, 79.2 mmol) in EtOH (300 mL) was added ethylhydrazine dihydrochloride (10.5 g, 79.2 mmol) and pyridine (25.0 g, 317 mmol). The suspension was stirred at room temperature for 16 h then concentrated under reduced pressure. The residue was diluted with water (400 mL) then extracted with EtOAc (200 mL×3). The organic layer was washed with brine (200 mL×2), dried over Na₂SO₄, filtered and concentrated under reduced pressure. The residue was purified by column chromatography (silica gel; 0-25% EtOAc:hexane) to give 5-(tert-butyl) 3-ethyl 1-ethyl-7-(trifluoromethyl)-1,4-dihydro-5H-pyrazolo[4,3-c]pyridine-3,5-dicarboxylate (8.20 g, 26.6% yield) as a yellow gum. (LC/MS) m/z (M+H)⁺=390.2. ¹H NMR (400 MHz, (CD₃)₂SO) δ 7.50 (s, 1H), 4.96 (s, 2H), 4.32-4.15 (m, 4H), 1.50 (s, 9H), 1.37-1.26 (m, 6H).

### Step 3. Preparation of rac-5-(tert-butyl) 3-ethyl (R)-1-ethyl-7-(trifluoromethyl)-1,4,6,7-tetrahydro-5H-pyrazolo[4,3-c]pyridine-3,5-dicarboxylate

A solution of 5-(tert-butyl) 3-ethyl 1-ethyl-7-(trifluoromethyl)-1,4-dihydro-5H-pyrazolo[4,3-c]pyridine-3,5-dicarboxylate (8.20 g, 21.1 mmol) in EtOH (164 mL) was delivered by a plunger pump at a flow rate of 1.00 mL/min into the flow reactor. Hydrogen gas from a high-pressure cylinder was introduced via a mass flow controller at 57.5 sccm and the system was maintained at 1 MPa. The liquid and gas streams were mixed with a T-shaped mixer and the combined solution was passed into a stainless-steel fixed-bed reactor (outer diameter: 9.525 mm (⅜ inch). The internal volume was 15.00 mL and packed with 5% Pd/Al₂O₃ (10.2 g) and held at 60.0 °C. The reaction mixture was collected after 3.73 h then concentrated by reduced pressure. The residue was purified by column chromatography (silica gel; 0-10% MeOH: DCM) to give rac-5-(tert-butyl) 3-ethyl (R)-1-ethyl-7-(trifluoromethyl)-1,4,6,7-tetrahydro-5H-pyrazolo[4,3-c]pyridine-3,5-dicarboxylate (6.36 g, 77.2% yield) as an off-yellow solid. (LC/MS) m/z (M+H)⁺=392.1. ¹H NMR (400 MHz, (CD₃)₂SO) δ 5.11-4.89 (m, 1H), 4.68-4.50 (m, 1H), 4.44-3.98 (m, 6H), 3.29-3.06 (m, 1H), 1.44-1.40 (m, 9H), 1.37 (t, 3H), 1.30 (t, 3H).

### Step 4. Preparation of rel-5-(tert-Butyl) 3-ethyl (R)-1-ethyl-7-(trifluoromethyl)-1,4,6,7-tetrahydro-5H-pyrazolo[4,3-c]pyridine-3,5-dicarboxylate and rel-5-(tert-Butyl) 3-ethyl (S)-1-ethyl-7-(trifluoromethyl)-1,4,6,7-tetrahydro-5H-pyrazolo[4,3-c]pyridine-3,5-dicarboxylate

rac-5-(tert-butyl) 3-ethyl (R)-1-ethyl-7-(trifluoromethyl)-1,4,6,7-tetrahydro-5H-pyrazolo[4,3-c]pyridine-3,5-dicarboxylate (3.00 g, 7.66 mmol) was separated into its component isomers using SFC purification (Chiral Technologies IG 250 mm×30 mm×5 μm column; Mobile phase A: CO₂; Mobile phase B: EtOH (0.2% [7N NH₃ in MeOH]); isocratic: 10% of Mobile Phase B; flow rate: 100 mL/min; column temperature: 40 °C; back pressure: 100 bar) to give the first eluting isomer: F68 (1.87 g, crude) as a waxy solid and the second eluting isomer: F69 (1.87 g, crude) as a waxy solid. The solids were used directly into the next step without further purification. The chiral purity was determined using SFC analytical method on Chiral Technologies IG 250 mm×4.6 mm×5 μm column; Mobile phase A: CO₂; Mobile phase B: Mobile phase B: EtOH (0.2% [7N NH₃ in MeOH]); gradient: held at 5% of Mobile phase B for 1.00 min then 5-80% of Mobile Phase B over 4.50 min then held at 80% of Mobile Phase B over 2.00 min then 80-5% of Mobile Phase B over 0.5 min; flow rate: 3.0 mL/min; backpressure: 100 bar; column temperature: 40 °C to provide F68: peak 1 ((SFC-HPLC) retention time: 4.062 min, 100% ee) and F69: peak 2 ((SFC-HPLC) retention time: 4.568 min, 100% ee).

### Preparation F70

### rel-Ethyl (R)-1-ethyl-7-(trifluoromethyl)-4,5,6,7-tetrahydro-1H-pyrazolo[4,3-c]pyridine-3-carboxylate hydrochloride

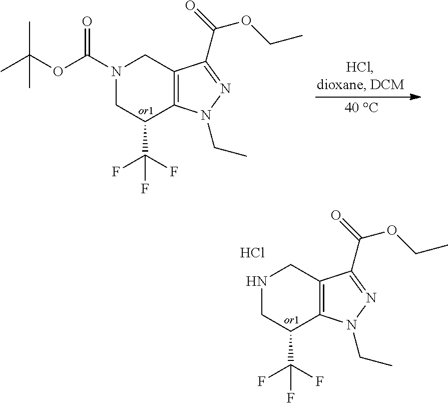 [SMILES: CCOC(=O)c1nn(CC)c2c1CN(C(=O)OC(C)(C)C)C[C@H]2C(F)(F)F ; Cl ; CCOC(=O)c1nn(CC)c2c1CNC[C@H]2C(F)(F)F]

### Step 1. Preparation of rel-ethyl (R)-1-ethyl-7-(trifluoromethyl)-4,5,6,7-tetrahydro-1H-pyrazolo[4,3-c]pyridine-3-carboxylate hydrochloride

To a reaction mixture of rel-5-(tert-Butyl) 3-ethyl (R)-1-ethyl-7-(trifluoromethyl)-1,4,6,7-tetrahydro-5H-pyrazolo[4,3-c]pyridine-3,5-dicarboxylate (second eluting isomer F69, 1.87 g, 4.78 mmol) in DCM (20 mL) was added 4M HCl in dioxane (871 mg, 5.97 mL, 23.9 mmol). The reaction was stirred at 40 °C for 1 h then concentrated by reduced pressure to give rel-ethyl (R)-1-ethyl-7-(trifluoromethyl)-4,5,6,7-tetrahydro-1H-pyrazolo[4,3-c]pyridine-3-carboxylate hydrochloride (1.57 g, crude) as a yellow oil. ¹H NMR (400 MHz, (CD₃)₂SO) δ 10.01 (br s, 1H), 8.44 (br s, 1H), 4.83-4.75 (m, 1H), 4.41-4.14 (m, 6H), 3.92 (d, 1H), 3.51 (dd, 1H), 3.45-3.34 (m, 1H), 1.39 (t, 3H), 1.30 (t, 3H).

### Preparation F71

### rel-Ethyl (S)-1-ethyl-7-(trifluoromethyl)-4,5,6,7-tetrahydro-1H-pyrazolo[4,3-c]pyridine-3-carboxylate hydrochloride

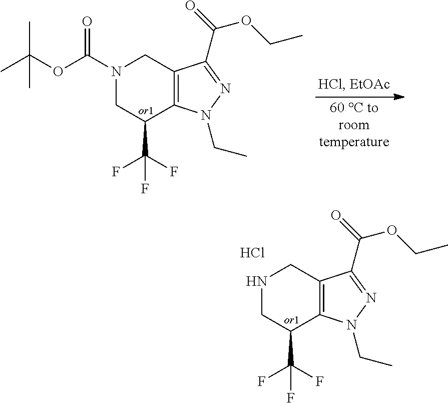 [SMILES: CCOC(=O)c1nn(CC)c2c1CN(C(=O)OC(C)(C)C)C[C@@H]2C(F)(F)F ; Cl ; CCOC(=O)c1nn(CC)c2c1CNC[C@@H]2C(F)(F)F]

### Step 1. Preparation of rel-ethyl (S)-1-ethyl-7-(trifluoromethyl)-4,5,6,7-tetrahydro-1H-pyrazolo[4,3-c]pyridine-3-carboxylate hydrochloride

To a reaction mixture of rel-5-(tert-Butyl) 3-ethyl (S)-1-ethyl-7-(trifluoromethyl)-1,4,6,7-tetrahydro-5H-pyrazolo[4,3-c]pyridine-3,5-dicarboxylate (first eluting isomer F68, 1.2 g, 3.1 mmol) in 2M HCl in EtOAc (0.89 g, 12 mL, 25 mmol). The reaction was stirred at room temperature overnight then stirred at 60 °C for 2 h. The reaction mixture was concentrated by reduced pressure to give rel-ethyl (S)-1-ethyl-7-(trifluoromethyl)-4,5,6,7-tetrahydro-1H-pyrazolo[4,3-c]pyridine-3-carboxylate hydrochloride (0.73 g, crude) as a yellow foam. The yellow foam was used directly into the next step without further purification. ¹H NMR (600 MHz, (CD₃)₂SO) δ 11.88 (br s, 1H), 11.00 (br s, 1H), 8.83 (br s, 1H), 4.82 (s, 1H), 4.42-4.13 (m, 6H), 3.88-3.83 (m, 1H), 3.76-3.60 (m, 1H), 3.58-3.55 (m, 1H), 1.39 (s, 3H), 1.30 (s, 3H).

### Preparation F72

### (S)-7-Methyl-4,5,6,7-tetrahydro-1H-pyrazolo[4,3-c]pyridine-3-carboxylic acid hydrochloride

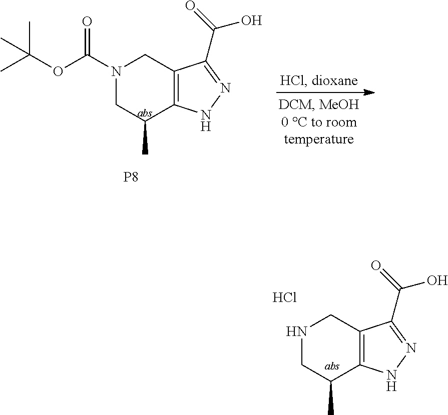 [SMILES: C[C@H]1CN(C(=O)OC(C)(C)C)Cc2c(C(=O)O)n[nH]c21 ; "P8" ; Cl ; C[C@H]1CNCc2c(C(=O)O)n[nH]c21]

### Step 1. Preparation of (S)-7-methyl-4,5,6,7-tetrahydro-1H-pyrazolo[4,3-c]pyridine-3-carboxylic acid hydrochloride

At 0 °C, to the solution of (S)-5-(tert-butoxycarbonyl)-7-methyl-4,5,6,7-tetrahydro-1H-pyrazolo[4,3-c]pyridine-3-carboxylic acid (10.0 g, 35.5 mmol) in DCM (50 mL) and MeOH (5 mL) was added HCl in dioxane (100 mL). The reaction mixture was stirred at room temperature for 1.5 h then concentrated by reduced pressure. The residue was diluted with H₂O (20 mL) then lyophilized to give (S)-7-methyl-4,5,6,7-tetrahydro-1H-pyrazolo[4,3-c]pyridine-3-carboxylic acid hydrochloride (7.73 g, crude) as a white solid. The solid was used directly in the next step without further purification. (LC/MS) m/z (M+H)⁺=182.2.

### Preparation F73

### (S)-6-(3-((4-(3,3-Dimethylureido)benzyl)carbamoyl)-1-isopropyl-7-methyl-1,4,6,7-tetrahydro-5H-pyrazolo[4,3-c]pyridin-5-yl)nicotinic acid trifluoroacetate

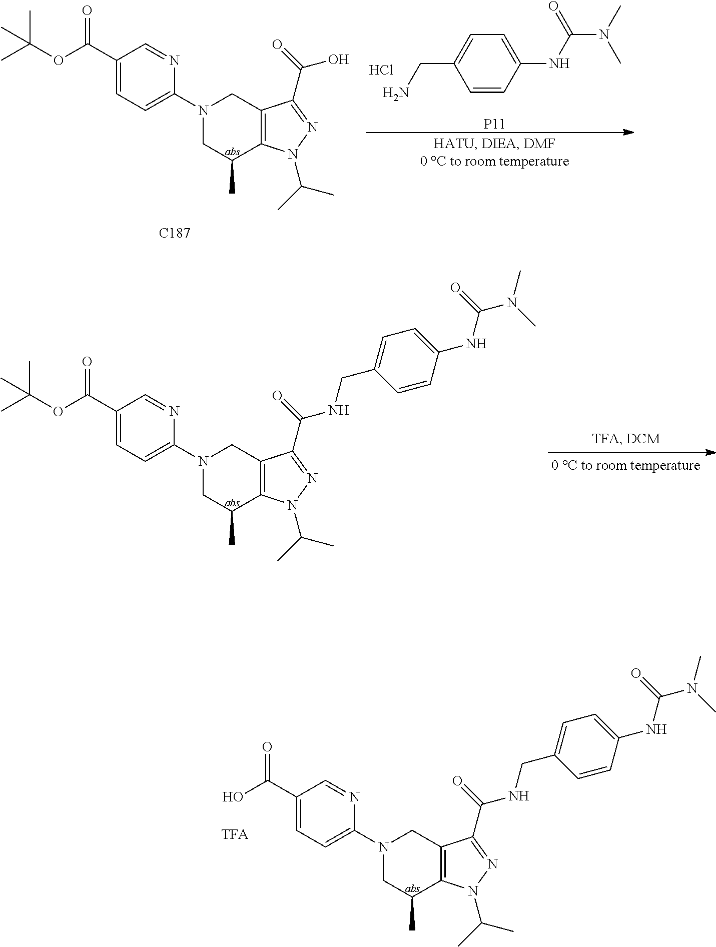

### Step 1. Preparation of tert-butyl (S)-6-(3-((4-(3,3-dimethylureido)benzyl)carbamoyl)-1-isopropyl-7-methyl-1,4,6,7-tetrahydro-5H-pyrazolo[4,3-c]pyridin-5-yl)nicotinate

To a solution of (S)-5-(5-(tert-butoxycarbonyl)pyridin-2-yl)-1-isopropyl-7-methyl-4,5,6,7-tetrahydro-1H-pyrazolo[4,3-c]pyridine-3-carboxylic acid (10.0 g, 25.0 mmol) and 3-(4-(aminomethyl)phenyl)-1,1-dimethylurea hydrochloride (5.74 g, 25.0 mmol) in DMF (100 mL) were added HATU (9.97 g, 26.2 mmol) and DIEA (12.9 g, 99.9 mmol) from 0 °C to room temperature. The reaction mixture was stirred at room temperature under nitrogen gas for 2 h. The resulting mixture was slowly added to ice-water (800 mL), which caused the solid to precipitate. The mixture was filtered then the filter cake was dissolved in EtOAc (150 mL). The organic layer was separated and washed with brine (1×) then dried over Na₂SO₄ and concentrated in vacuo. The resulting white solid was lyophilized to afford tert-butyl (S)-6-(3-((4-(3,3-dimethylureido)benzyl)carbamoyl)-1-isopropyl-7-methyl-1,4,6,7-tetrahydro-5H-pyrazolo[4,3-c]pyridin-5-yl)nicotinate (13.0 g, crude) as a white solid. The solid was used directly in the next step without further purification. (LC/MS) m/z (M+H)⁺=576.6. ¹H NMR (400 MHz, (CD₃)₂SO) δ 8.59 (d, 1H), 8.35 (t, 1H), 8.21 (s, 1H), 7.88 (dd, 1H), 7.41-7.33 (m, 2H), 7.19-7.11 (m, 2H), 6.89 (d, 1H), 5.17 (d, 1H), 4.56-4.46 (m, 2H), 4.38-4.21 (m, 3H), 3.31-3.20 (m, 2H), 2.90-2.84 (m, 6H), 1.49 (s, 9H), 1.39 (dd, 6H), 1.17-1.11 (m, 3H).

### Step 2. Preparation of (S)-6-(3-((4-(3,3-dimethylureido)benzyl)carbamoyl)-1-isopropyl-7-methyl-1,4,6,7-tetrahydro-5H-pyrazolo[4,3-c]pyridin-5-yl)nicotinic acid trifluoroacetate

At 0 °C, to a solution of tert-butyl (S)-6-(3-((4-(3,3-dimethylureido)benzyl)carbamoyl)-1-isopropyl-7-methyl-1,4,6,7-tetrahydro-5H-pyrazolo[4,3-c]pyridin-5-yl)nicotinate (13.0 g, 22.6 mmol) in DCM (20 mL) was added TFA (40 mL). The reaction mixture was stirred at room temperature for 3.5 h then concentrated in vacuo. The yellow gum was lyophilized to give a yellow solid then diluted with MTBE (150 mL). The suspension was stirred at room temperature for 2 h then filtered. The filter cake was washed with MTBE (50 mL) then concentrated in vacuo to give (S)-6-(3-((4-(3,3-dimethylureido)benzyl)carbamoyl)-1-isopropyl-7-methyl-1,4,6,7-tetrahydro-5H-pyrazoo[4,3-c]pyridin-5-yl)nicotinic acid trifluoroacetate (13.0 g, crude) as a white solid. The solid was used directly in the next step without further purification. (LC/MS) m/z (M+H)⁺=520.3. ¹H NMR (400 MHz, (CD₃)₂SO) 6 8.63 (d, 1H), 8.38 (t, 1H), 8.23 (s, 1H), 7.97 (dd, 1H), 7.43-7.35 (m, 2H), 7.21-7.14 (m, 2H), 6.97 (d, 1H), 5.18 (d, 1H), 4.59-4.46 (m, 2H), 4.40-4.26 (m, 4H), 3.44-3.35 (m, 1H), 3.32-3.22 (m, 1H) 2.90 (s, 6H), 1.42 (dd, 6H), 1.18 (d, 3H).

### Preparation F74

### (S)-6-(3-((4-(3,3-Dimethylureido)benzyl)carbamoyl)-1-isopropyl-7-methyl-1,4,6,7-tetrahydro-5H-pyrazolo[4,3-c]pyridin-5-yl)nicotinic acid hydrochloride

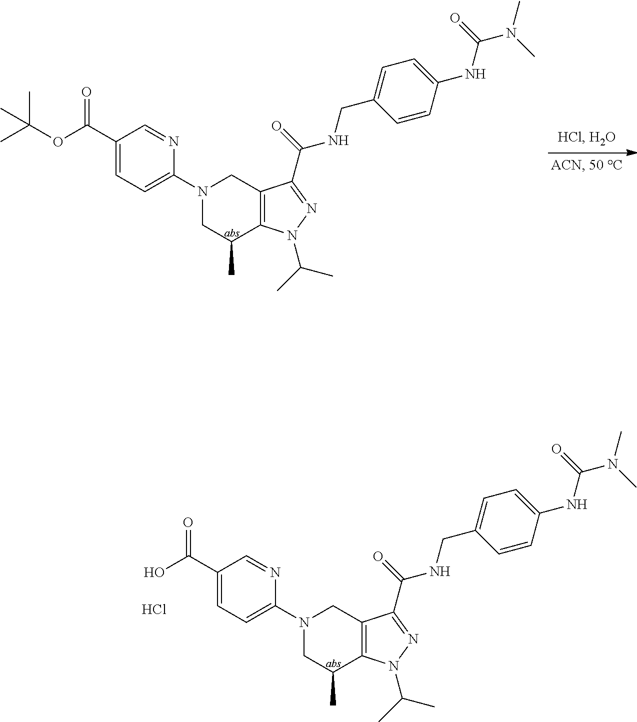 [SMILES: CC(C)n1nc(C(=O)NCc2ccc(NC(=O)N(C)C)cc2)c2c1[C@@H](C)CN(c1ccc(C(=O)OC(C)(C)C)cn1)C2 ; Cl ; CC(C)n1nc(C(=O)NCc2ccc(NC(=O)N(C)C)cc2)c2c1[C@@H](C)CN(c1ccc(C(=O)O)cn1)C2]

### Step 1. Preparation of (S)-6-(3-((4-(3,3-dimethylureido)benzyl)carbamoyl)-1-isopropyl-7-methyl-1,4,6,7-tetrahydro-5H-pyrazolo[4,3-c]pyridin-5-yl)nicotinic acid hydrochloride

To a round bottom flask with stir bar was added tert-butyl (S)-6-(3-((4-(3,3-dimethylureido)benzyl)carbamoyl)-1-isopropyl-7-methyl-1,4,6,7-tetrahydro-5H-pyrazoo[4,3-c]pyridin-5-yl)nicotinate (5.60 g, 9.73 mmol) and ACN (40 mL). To the reaction was added 12M aqueous HCl (4.0 mL) which caused solid to precipitate out of solution. The suspension was stirred at 50 °C overnight then concentrated by reduced pressure to give (S)-6-(3-((4-(3,3-dimethylureido)benzyl)carbamoyl)-1-isopropyl-7-methyl-1,4,6,7-tetrahydro-5H-pyrazoo[4,3-c]pyridin-5-yl)nicotinic acid hydrochloride (4.95 g, crude) as a light-brown foam. The foam was used directly 5 into the next step without further purification. (LC/MS) m/z (M+H)⁺=520.3. ¹H NMR (600 MHz, (CD₃)₂SO) δ 9.85 (s, 1H), 8.49 (d, 1H), 8.46-8.38 (m, 1H), 8.30 (s, 1H), 8.14 (dd, 1H), 7.43-7.38 (m, 2H), 7.34-7.27 (m, 1H), 7.19-7.15 (m, 2H), 5.14-5.08 (m, 1H), 4.63-4.47 (m, 2H), 4.40-4.29 (m, 2H), 3.61-3.53 (m, 2H), 3.38-3.33 (m, 1H), 3.14-2.95 (m, 1H), 2.90 (s, 6H), 1.41 (dd, 6H), 1.33-1.19 (m, 3H)(desired peaks only).

### Examples E1-E732

### Example E1

### (7S)-5-(5-(Dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide (E1)

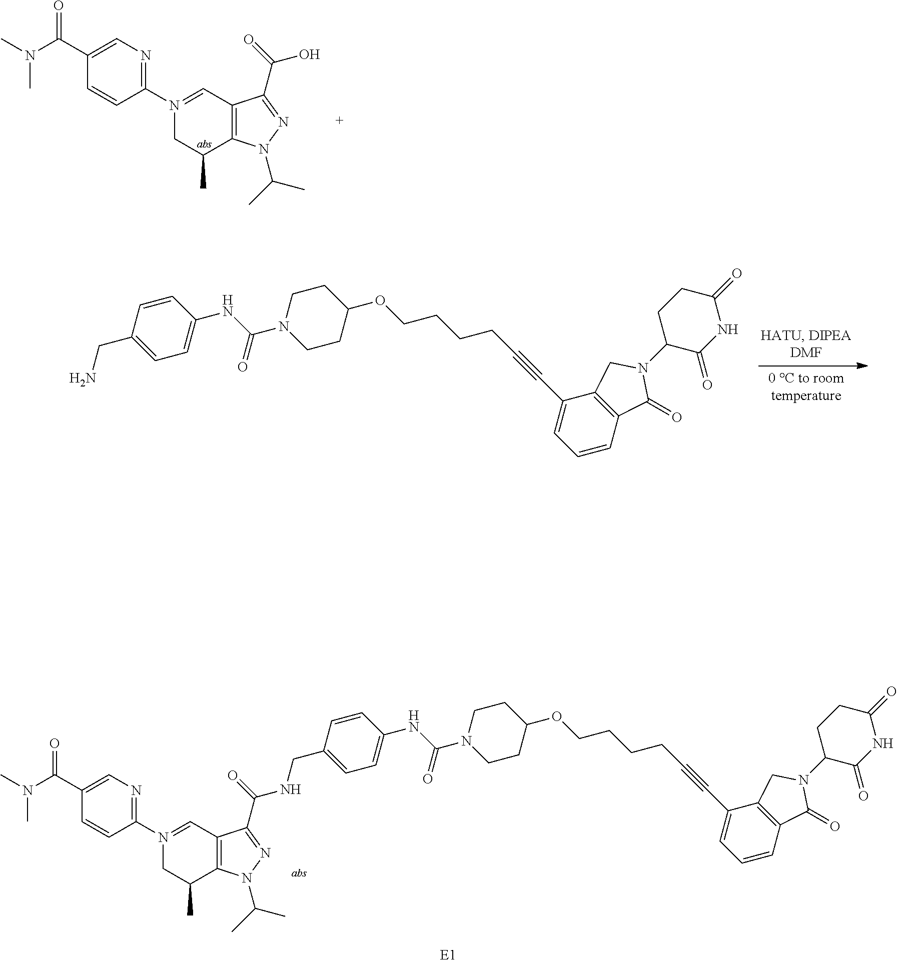

### Step 1. Preparation of (7S)-5-(5-(dimethylcarbamoyl)pyridin-2-yl)-N-(4-(4-((6-(2-(2,6-dioxopiperidin-3-yl)-1-oxoisoindolin-4-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide (E1)

To a solution of (S)-5-(5-(dimethylcarbamoyl)pyridin-2-yl)-1-isopropyl-7-methyl-4,5,6,7-tetrahydro-1H-pyrazolo[4,3-c]pyridine-3-carboxylic acid (70.4 mg, 0.190 mmol) in DMF (1.5 mL) was added HATU (75.7 mg, 0.199 mmol). The reaction mixture was stirred at room temperature for 10 min then cooled to 0 °C before N-(4-(aminomethyl)phenyl)-4-((6-(2-(2,6-dioxopiperidin-3-yl)-1-oxoisoindolin-4-yl)hex-5-yn-1-yl)oxy)piperidine-1-carboxamide trifluoroacetate (0.130 g, 0.190 mmol) and DIEA (98.0 mg, 0.758 mmol) were added dropwise. The solution was warmed to room temperature then stirred for 16 hours. After the stir, the solution was diluted with DMF and purified by reverse phase HPLC (C₁₈ 150 mm×40 mm×5 μm column, Mobile Phase A: H₂O (NH₄OH—NH₄HCO₃)/Mobile Phase B: ACN, gradient: 28 to 68% of Mobile Phase B over 9 min, 100% of Mobile Phase B held 2 min, flow rate: 60 mL/min) to provide E1 (57.7 mg, 32.9% yield) as a white solid. (LC/MS) m/z (M+H)⁺=925.3. ¹H NMR (400 MHz, (CD₃)₂SO) δ 10.99 (s, 1H), 8.45 (s, 1H), 8.35 (t, 1H), 8.23 (d, 1H), 7.73-7.67 (m, 1H), 7.67-7.59 (m, 2H), 7.51 (t, 1H), 7.40-7.33 (m, 2H), 7.20-7.13 (m, 2H), 6.88 (d, 1H), 5.18-5.04 (m, 2H), 4.58-4.18 (m, 7H), 3.82-3.72 (m, 2H), 3.50-3.45 (m, 2H), 3.26-3.20 (m, 2H), 3.13-3.03 (m, 2H), 3.01-2.94 (m, 6H), 2.93-2.85 (m, 1H), 2.68-2.54 (m, 1H), 2.52-2.44 (m, 3H), 2.43-2.36 (m, 1H), 2.04-1.97 (m, 1H), 1.86-1.78 (m, 2H), 1.67-1.60 (m, 4H), 1.46-1.33 (m, 8H), 1.19 (d, 3H).

### Example E2

### (S)-5-(4-(Dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide formate (E2)

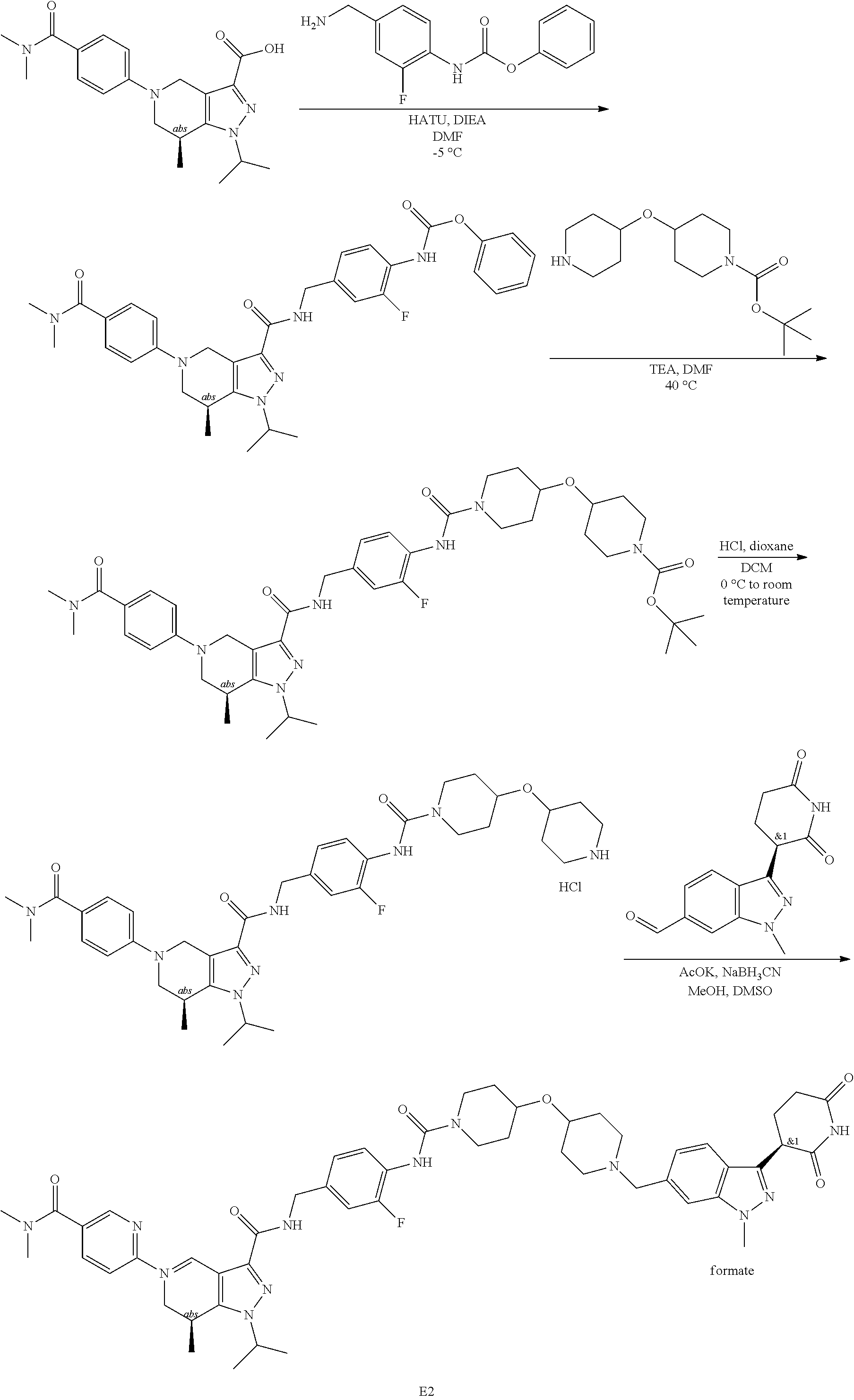

### Step 1. Preparation of phenyl (S)-(4-((5-(4-(dimethylcarbamoyl)phenyl)-1-isopropyl-7-methyl-4,5,6,7-tetrahydro-1H-pyrazolo[4,3-c]pyridine-3-carboxamido)methyl)-2-fluorophenyl)carbamate

At −5 °C, to a reaction mixture of (S)-5-(4-(dimethylcarbamoyl)phenyl)-1-isopropyl-7-methyl-4,5,6,7-tetrahydro-1H-pyrazolo[4,3-c]pyridine-3-carboxylic acid (1.64 g, 4.41 mmol) and phenyl (4-(aminomethyl)-2-fluorophenyl)carbamate hydrochloride (1.31 g, 4.41 mmol) in DMF (13 mL) was added HATU (1.76 g, 4.63 mmol). The reaction mixture was stirred at −5 °C for 10 min then DIEA (2.28 g, 17.7 mmol) was added. The suspension was stirred at −5 °C for 1.5 hours then was poured into H₂O, filtered and lyophilized to provide phenyl (S)-(4-((5-(4-(dimethylcarbamoyl)phenyl)-1-isopropyl-7-methyl-4,5,6,7-tetrahydro-1H-pyrazolo[4,3-c]pyridine-3-carboxamido)methyl)-2-fluorophenyl)carbamate (2.56 g, 94.7% yield) as a white solid. (LC/MS) m/z (M+H)⁺=613.1. ¹H NMR (400 MHz, (CD₃)₂SO) δ 9.88 (s, 1H), 8.55 (t, 1H), 7.57 (t, 1H), 7.46-7.38 (m, 2H), 7.37-7.30 (m, 2H), 7.27-7.12 (m, 5H), 7.00-6.94 (m, 2H), 4.61-4.50 (m, 2H), 4.40 (d, 2H), 4.11 (d, 1H), 3.82 (d, 1H), 3.26-3.16 (m, 1H), 2.96 (s, 6H), 2.92-2.86 (m, 1H), 1.49-1.37 (m, 6H), 1.32-1.26 (m, 3H).

### Step 2. Preparation of tert-butyl (S)-4-((1-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)-2-fluorophenyl)carbamoyl)piperidin-4-yl)oxy)piperidine-1-carboxylate

To a solution of phenyl (S)-(4-((5-(4-(dimethylcarbamoyl)phenyl)-1-isopropyl-7-methyl-4,5,6,7-tetrahydro-1H-pyrazolo[4,3-c]pyridine-3-carboxamido)methyl)-2-fluorophenyl)carbamate (5.00 g, 8.16 mmol) in DMF (50 mL) were added TEA (3.30 g, 32.6 mmol) and tert-butyl 4-(piperidin-4-yloxy)piperidine-1-carboxylate (CAS: 845305-83-1; 2.37 g, 8.32 mmol). The reaction mixture was stirred at 40 °C for 2 hours then was poured into H₂O (300 mL) which caused white solid to precipitate. The suspension was filtered, and the filter cake was collected then purified by column chromatography (silica gel; 0-10% MeOH: DCM) to provide tert-butyl (S)-4-((1-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)-2-fluorophenyl)carbamoyl)piperidin-4-yl)oxy)piperidine-1-carboxylate (5.65 g, 86.2% yield) as a yellow solid. (LC/MS) m/z (M+H)⁺=803.2. ¹H NMR (400 MHz, (CD₃)₂SO) δ 8.51 (t, 1H), 8.25-8.20 (m, 1H), 7.36-7.25 (m, 3H), 7.14-6.93 (m, 4H), 4.61-4.50 (m, 2H), 4.38 (d, 2H), 4.15-4.04 (m, 1H), 3.86-3.71 (m, 3H), 3.69-3.56 (m, 4H), 3.33-3.18 (m, 3H), 3.14-2.99 (m, 3H), 2.98-2.93 (m, 6H), 1.83-1.70 (m, 4H), 1.51-1.24 (m, 22H).

### Step 3. Preparation of (S)-5-(4-(dimethylcarbamoyl)phenyl)-N-(3-fluoro-4-(4-(piperidin-4-yloxy)piperidine-1-carboxamido)benzyl)-1-isopropyl-7-methyl-4,5,6,7-tetrahydro-1H-pyrazolo[4,3-c]pyridine-3-carboxamide hydrochloride

At 0 °C, to a solution of tert-butyl (S)-4-((1-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)-2-fluorophenyl)carbamoyl)piperidin-4-yl)oxy)piperidine-1-carboxylate (5.65 g, 7.04 mmol) in DCM (60 mL) was added 0.04M HCl in dioxane (120 mL). The reaction mixture was warmed to room temperature and stirred for 16 hours then concentrated in vacuo to provide (S)-5-(4-(dimethylcarbamoyl)phenyl)-N-(3-fluoro-4-(4-(piperidin-4-yloxy)piperidine-1-carboxamido)benzyl)-1-isopropyl-7-methyl-4,5,6,7-tetrahydro-1H-pyrazolo[4,3-c]pyridine-3-carboxamide hydrochloride (5.34 g, crude) as a yellow solid. The solid was used directly in the next step without further purification. (LC/MS) m/z ((M+H)/2)+=352.4. ¹H NMR (400 MHz, (CD₃)₂SO) δ 9.16-9.06 (m, 2H), 8.52 (t, 1H), 8.29 (s, 1H), 7.39-7.26 (m, 4H), 7.13-6.97 (m, 3H), 4.61-4.49 (m, 2H), 4.37 (d, 2H), 4.12 (d, 1H), 3.84-3.69 (m, 4H), 3.67-3.58 (m, 1H), 3.34-3.18 (m, 2H), 3.15-3.04 (m, 4H), 2.97-2.93 (m, 7H), 1.98-1.88 (m, 2H), 1.84-1.75 (m, 2H), 1.73-1.61 (m, 2H), 1.49-1.36 (m, 8H), 1.29 (d, 3H).

### Step 4. Preparation of (S)-5-(4-(dimethylcarbamoyl)phenyl)-N-(4-(4-((1-((3-((RS)-2,6-dioxopiperidin-3-yl)-1-methyl-1H-indazol-6-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide formate (E2)

To a solution of (S)-5-(4-(dimethylcarbamoyl)phenyl)-N-(3-fluoro-4-(4-(piperidin-4-yloxy)piperidine-1-carboxamido)benzyl)-1-isopropyl-7-methyl-4,5,6,7-tetrahydro-1H-pyrazolo[4,3-c]pyridine-3-carboxamide hydrochloride (0.777 g, 1.11 mmol) and rac-(R)-3-(2,6-dioxopiperidin-3-yl)-1-methyl-1H-indazole-6-carbaldehyde (CAS: 2925099-25-6; 0.300 g, 1.11 mmol) in MeOH (5 mL) and DMSO (10 mL) was added AcOK (0.217 g, 2.21 mmol) and NaBH₃CN (0.208 g, 3.32 mmol). The reaction mixture was stirred at room temperature for 16 hours. The suspension was filtered, and the filter cake was washed with ACN (5 mL). The filtrate was purified by reverse phase HPLC (WePure Biotech XP tC₁₈ 150 mm×40 mm×7 μm column; Mobile phase A: H₂O (0.225% formic acid); Mobile Phase B: ACN; gradient: 10-40% of Mobile Phase B in 12 min; flow rate: 60 mL/min) and lyophilized to provide E2 (0.168 g, 15.1% yield) as a white solid. (LC/MS) m/z (M+H)⁺=958.3; ¹H NMR (400 MHz, (CD₃)₂SO) δ 10.91 (s, 1H), 8.52 (t, 1H), 8.24 (s, 1H), 8.15 (s, 1H), 7.66 (d, 1H), 7.52 (s, 1H), 7.36-7.25 (m, 3H), 7.15-7.02 (m, 3H), 6.97 (d, 2H), 4.61-4.50 (m, 2H), 4.40-4.31 (m, 3H), 4.10 (d, 1H), 4.02-3.92 (m, 4H), 3.86-3.67 (m, 8H), 3.32-3.17 (m, 2H), 3.07 (t, 3H), 2.96 (s, 6H), 2.80-2.75 (m, 2H), 2.72-2.54 (m, 2H), 2.30-2.21 (m, 2H), 1.85-1.73 (m, 4H), 1.53-1.23 (m, 11H).

### Example E3

### (S)-5-(4-(Dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide (E3)

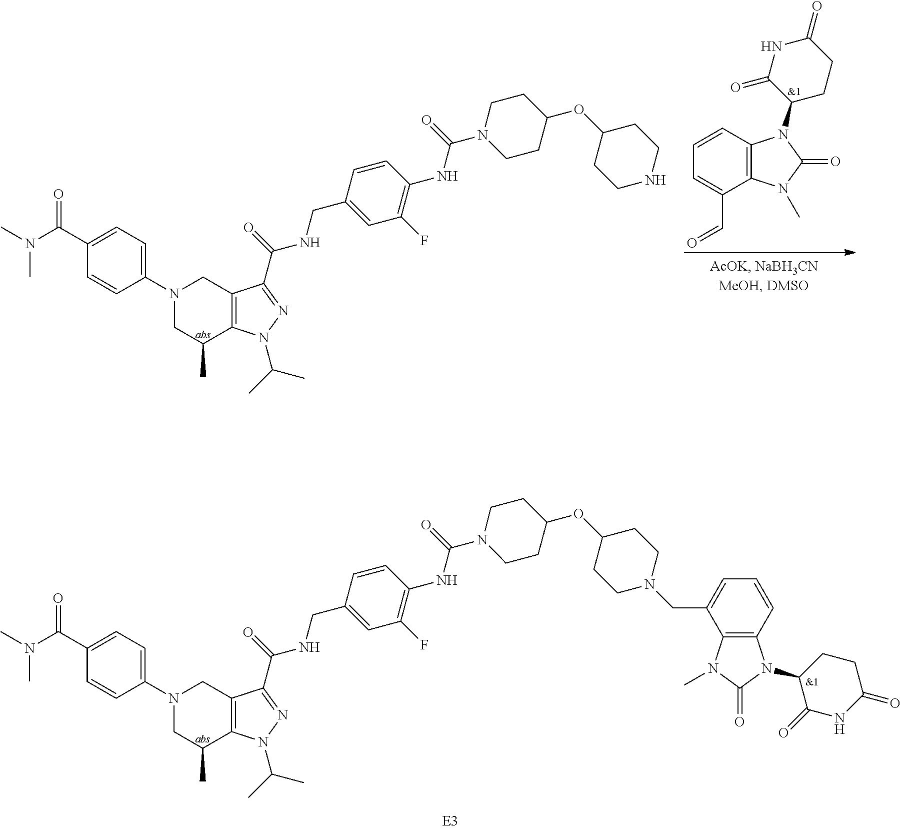

### Step 1. Preparation of (S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide (E3)

To a solution of (S)-5-(4-(dimethylcarbamoyl)phenyl)-N-(3-fluoro-4-(4-(piperidin-4-yloxy)piperidine-1-carboxamido)benzyl)-1-isopropyl-7-methyl-4,5,6,7-tetrahydro-1H-pyrazolo[4,3-c]pyridine-3-carboxamide hydrochloride (0.080 g, 0.097 mmol) and rac-(R)-1-(2,6-dioxopiperidin-3-yl)-3-methyl-2-oxo-2,3-dihydro-1H-benzo[d]imidazole-4-carbaldehyde (CAS: 2360518-87-0; 0.028 g, 0.097 mmol) in MeOH (0.8 mL) and DMSO (1.2 mL) was added AcOK (0.019 g, 0.19 mmol) and NaBH₃CN (0.018 g, 0.29 mmol). The reaction mixture was stirred at room temperature for 16 hours. The suspension was filtered. The filtrate was diluted with MeOH (1 mL) then purified by reverse phase HPLC (Phenomenex Gemini NX C₁₈ 150 mm×30 mm×5 μm column; Mobile phase A: H₂O (0.225% formic acid); Mobile Phase B: ACN; gradient: 15-55% of Mobile Phase B over 10 min; held at 100% of Mobile Phase B over 3 min; flow rate: 30 mL/min) and lyophilized to provide E3 (22 mg, 24% yield) as a white solid. (LC/MS) m/z (M+H)⁺=974.5; ¹H NMR (400 MHz, (CD₃)₂SO) δ 11.09 (s, 1H), 8.49 (t, 1H), 8.21 (s, 1H), 7.36-7.26 (m, 3H), 7.14-7.01 (m, 3H), 7.00-6.91 (m, 3H), 6.87 (d, 1H), 5.37 (dd, 1H), 4.61-4.50 (m, 2H), 4.38 (d, 2H), 4.11 (d, 1H), 3.85-3.73 (m, 3H), 3.67 (s, 3H), 3.64-3.59 (m, 3H), 3.51-3.44 (m, 1H), 3.28-3.17 (m, 3H), 3.12-3.02 (m, 2H), 2.96-2.82 (m, 7H), 2.77-2.57 (m, 4H), 2.20-2.08 (m, 2H), 2.05-1.95 (m, 1H), 1.83-1.74 (m, 4H), 1.49-1.40 (m, 6H), 1.39-1.32 (m, 3H), 1.31-1.26 (m, 3H).

### Example E4

### (7S)—N-(4-(3,3-Dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide (E4)

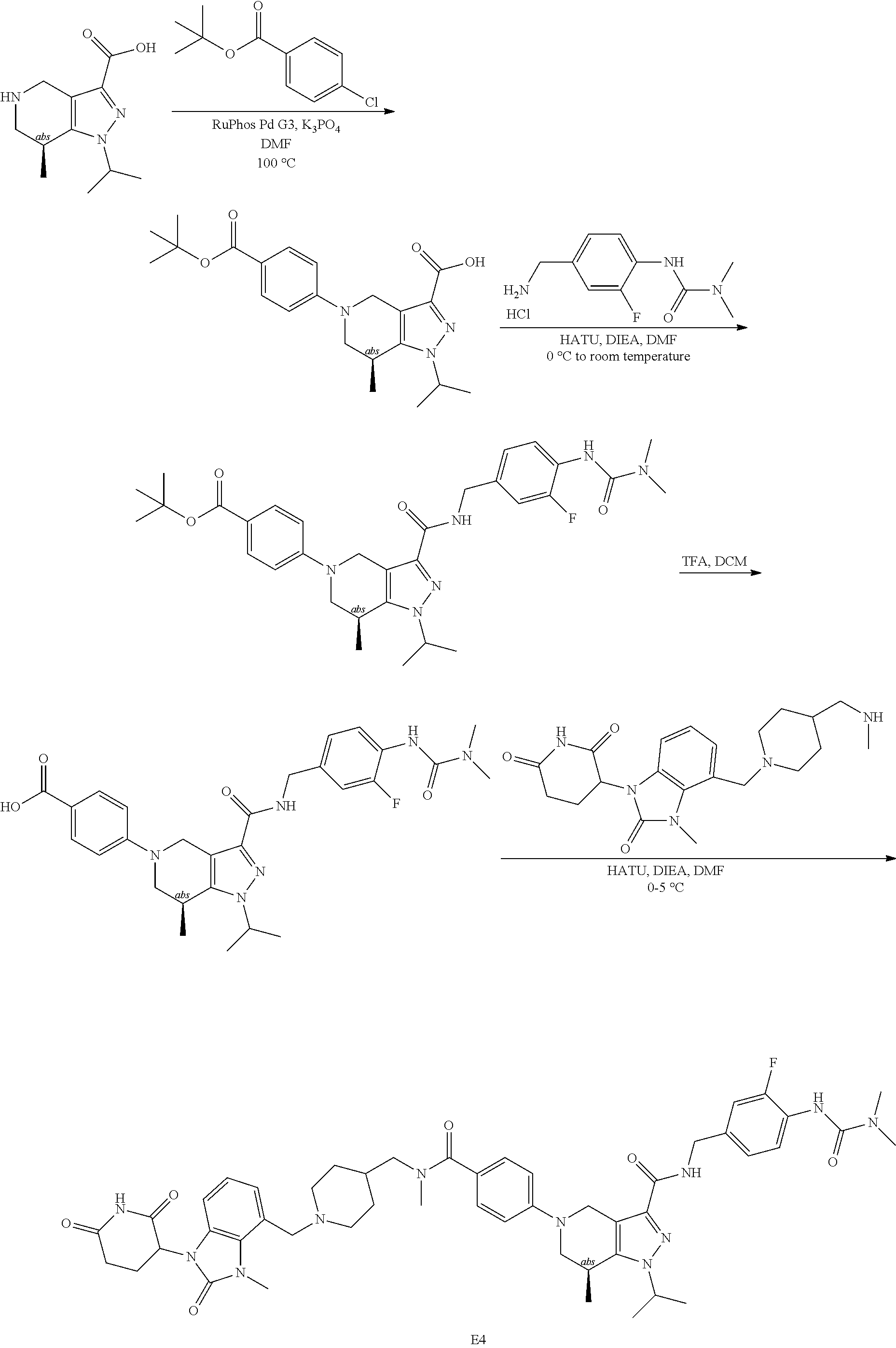

### Step 1. Preparation of (S)-5-(4-(tert-butoxycarbonyl)phenyl)-1-isopropyl-7-methyl-4,5,6,7-tetrahydro-1H-pyrazolo[4,3-c]pyridine-3-carboxylic acid

Under argon gas, a reaction mixture of (S)-1-isopropyl-7-methyl-4,5,6,7-tetrahydro-1H-pyrazolo[4,3-c]pyridine-3-carboxylic acid hydrochloride (0.400 g, 1.54 mmol), tert-butyl 4-chlorobenzoate (0.495 g, 1.92 mmol) and K₃PO₄ (1.31 g, 6.16 mmol) in DMF (8 mL) was added RuPhos Pd G3 (0.129 g, 0.154 mmol). The suspension was stirred at 100 °C under argon gas for 16 hours then poured into H₂O (40 mL). The mixture was extracted with EtOAc (20 mL×2). The organic layer was discarded. The aqueous layer was acidified with 1 N HCl to pH=−5-6 then extracted with EtOAc (20 mL×3). The combined organic layer was dried with Na₂SO₄, filtered, concentrated in vacuo and lyophilized to provide (S)-5-(4-(tert-butoxycarbonyl)phenyl)-1-isopropyl-7-methyl-4,5,6,7-tetrahydro-1H-pyrazolo[4,3-c]pyridine-3-carboxylic acid (0.430 g, crude) as a white solid. The solid was used directly in the next step without further purification. (LCMS) m/z (M+H)+=400.2.

### Step 2. Preparation of tert-butyl (S)-4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)benzoate

At 0-5 °C under nitrogen gas, to a reaction mixture of (S)-5-(4-(tert-butoxycarbonyl)phenyl)-1-isopropyl-7-methyl-4,5,6,7-tetrahydro-1H-pyrazolo[4,3-c]pyridine-3-carboxylic acid (0.430 g, 1.08 mmol) and HATU (409 mg, 1.08 mmol) in DMF (5 mL) was added 3-(4-(aminomethyl)-2-fluorophenyl)-1,1-dimethylurea hydrochloride (267 mg, 1.08 mmol) and DIEA (417 mg, 3.23 mmol). The reaction mixture was stirred at room temperature for 1 hour then poured into H₂O (50 mL) and filtered. The filter cake was collected then purified by column chromatography (silica gel, 0-10% MeOH: DCM) to provide tert-butyl (S)-4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)benzoate (0.600 g, 94.0% yield) as a white solid. (LCMS) m/z (M+H)⁺=593.4. ¹H NMR (400 MHz, (CD₃)₂SO) δ 8.53 (t, 1H), 8.00-7.93 (m, 1H), 7.78-7.71 (m, 2H), 7.35 (t, 1H), 7.14-6.96 (m, 4H), 4.66-4.50 (m, 2H), 4.38 (d, 2H), 4.19 (d, 1H), 3.92 (d, 1H), 3.26-3.18 (m, 1H), 2.91-2.84 (m, 7H), 1.54-1.49 (m, 9H), 1.43 (dd, 6H), 1.26 (d, 3H).

### Step 3. Preparation of (S)-4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)benzoic acid trifluoroacetate

To a solution of tert-butyl (S)-4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)benzoate (0.600 g, 1.01 mmol) in DCM (3 mL) was added TFA (7 mL). The reaction mixture was then stirred at room temperature for 3 hours then concentrated in vacuo and lyophilized to provide (S)-4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)benzoic acid trifluoroacetate (0.485 g, crude) as a yellow solid. The solid was used directly in the next step without further purification. (LC/MS) m/z (M+H)⁺=537.3. ¹H NMR (400 MHz, (CD₃)₂SO) δ 8.52 (t, 1H), 8.00-7.95 (m, 1H), 7.79 (d, 2H), 7.35 (t, 1H), 7.17-6.98 (m, 4H), 4.66-4.50 (m, 2H), 4.38 (d, 2H), 4.18 (d, 1H), 3.97-3.89 (m, 1H), 3.38-3.29 (m, 1H), 3.26-3.20 (m, 1H), 2.90 (s, 6H), 1.52-1.39 (m, 6H), 1.34-1.21 (m, 4H).

### Step 4. Preparation of (7S)—N-(4-(3,3-dimethylureido)-3-fluorobenzyl)-5-(4-(((1-((1-(2,6-dioxopiperidin-3-yl)-3-methyl-2-oxo-2,3-dihydro-1H-benzo[d]imidazol-4-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide (E4)

At 0-5 °C, to a reaction mixture of (S)-4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)benzoic acid trifluoroacetate (0.12 g, 0.20 mmol) in DMF (2 mL) was added HATU (77 mg, 0.20 mmol), DIEA (0.13 g, 1.0 mmol) and 3-(3-methyl-4-((4-((methylamino)methyl)piperidin-1-yl)methyl)-2-oxo-2,3-dihydro-1H-benzo[d]imidazol-1-yl)piperidine-2,6-dione bis(2,2,2-trifluoroacetate) (0.18 g, 0.20 mmol). The reaction mixture was stirred at 0-5 °C for 1.5 hours then purified by reverse phase HPLC (Boston Prime C₁₈ 150 mm×30 mm×5 μm column; Mobile phase A: H₂O (0.225% formic acid); Mobile Phase B: ACN; gradient: 13-43% of Mobile Phase B in 12 min; flow rate: 35 mL/min) and lyophilized to provide E4 (24 mg, 13% yield) as a white solid. (LC/MS) m/z (M+H)⁺=918.6. ¹H NMR (400 MHz, (CD₃)₂SO) δ 11.08 (s, 1H), 8.49 (t, 1H), 7.96 (s, 1H), 7.33 (t, 1H), 7.29-7.23 (m, 2H), 7.12-7.00 (m, 3H), 6.94 (d, 3H), 6.87-6.82 (m, 1H), 5.38-5.34 (m, 1H), 4.57-4.48 (m, 2H), 4.36 (d, 2H), 4.08 (d, 1H), 3.79 (d, 1H), 3.67-3.54 (m, 4H), 3.32-3.23 (m, 6H), 2.92 (s, 3H), 2.89 (s, 6H), 2.82-2.55 (m, 4H), 2.04-1.86 (m, 3H), 1.79-1.49 (m, 3H), 1.42 (dd, 6H), 1.27 (d, 3H), 1.15-0.80 (m, 2H).

### Example E9

### rac-(R)-5-(5-(Dimethylcarbamoyl)pyridin-2-yl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide (E9)

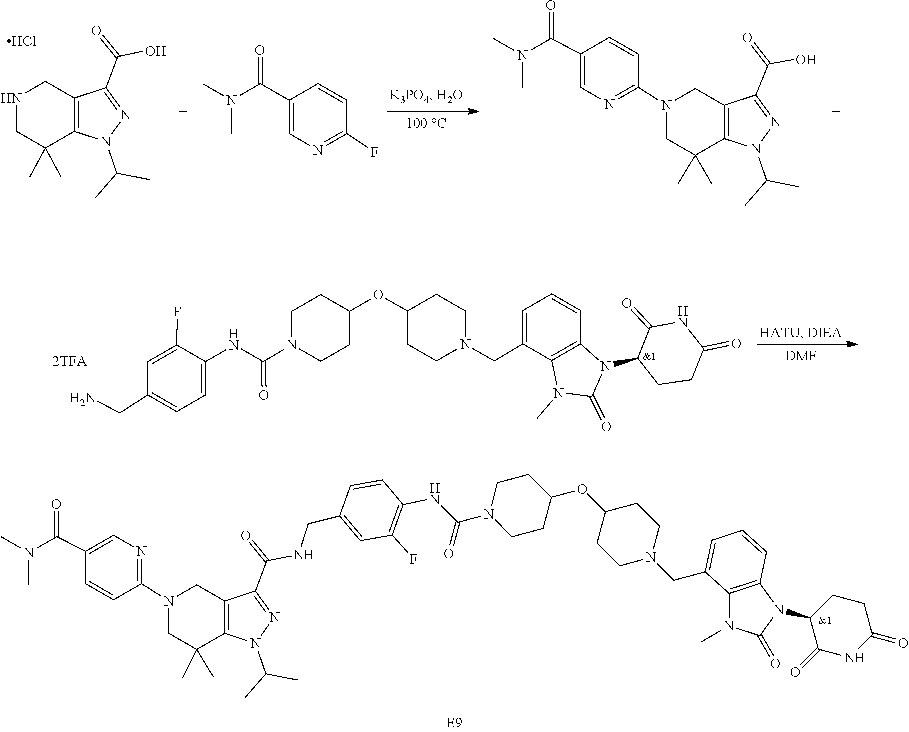

### Step 1: Preparation of 5-(5-(dimethylcarbamoyl)pyridin-2-yl)-1-isopropyl-7,7-dimethyl-4,5,6,7-tetrahydro-1H-pyrazolo[4,3-c]pyridine-3-carboxylic acid

To a mixture of 1-isopropyl-7,7-dimethyl-4,5,6,7-tetrahydro-1H-pyrazolo[4,3-c]pyridine-3-carboxylic acid hydrochloride (1500 mg, 5.48 mmol) and 6-fluoro-N,N-dimethylnicotinamide (921 mg, 5.48 mmol) in water (15 mL) was added potassium phosphate (1860 mg, 13.7 mmol). The reaction mixture was stirred at 100 °C for approximately 16 h. The reaction was diluted with water (10 mL) and extracted using EtOAc (3×10 mL). The combined organic layer was discarded. The aqueous layer was acidified to pH=~6 using 1M HCl, the resulting solid was filtered and lyophilized to provide first batch (714 mg, 33.8% yield) as a white solid.

The aqueous filtrate was acidified using 1 M HCl (~3 mL) to pH=6, then extracted with EtOAc (3×30 mL). The combined organic layers were concentrated in vacuo to provide second batch (416 mg, 19.7% yield). Both the batches were combined to provide 5-(5-(dimethylcarbamoyl)pyridin-2-yl)-1-isopropyl-7,7-dimethyl-4,5,6,7-tetrahydro-1H-pyrazolo[4,3-c]pyridine-3-carboxylic acid (1130 mg, crude) as a white solid. The solid was used directly in the next strep without further purification. (LCMS) m/z (M+H)⁺=386.3. ¹H NMR ((CD₃)₂SO, 400 MHz) δ 12.70 (br s, 1H), 8.24 (d, 1H), 7.63 (dd, 1H), 6.94 (d, 1H), 4.7-4.8 (m, 1H), 4.65 (s, 2H), 3.67 (s, 2H), 2.97 (s, 6H), 1.41 (d, 6H), 1.35 (s, 6H).

### Step 2: Preparation of rac-(R)-5-(5-(dimethylcarbamoyl)pyridin-2-yl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide (E9)

To a solution of 5-(5-(dimethylcarbamoyl)pyridin-2-yl)-1-isopropyl-7,7-dimethyl-4,5,6,7-tetrahydro-1H-pyrazolo[4,3-c]pyridine-3-carboxylic acid (50 mg, 0.10 mmol), rac-(R)—N-(4-(aminomethyl)-2-fluorophenyl)-4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamide bis(2,2,2-trifluoroacetate) (88.2 mg, 0.104 mmol) in DMF (2 mL) was added HATU (41.4 mg, 0.109 mmol) and DIEA (70 mg, 0.1 mL, 0.54 mmol). The mixture was stirred at room temperature for 2 h then diluted with ACN (2 mL), The suspension was filtered, and the filtrate was purified by reverse phase HPLC (WePure Biotech XP tC₁₈ 150 mm×30 mm×7 μm column; Mobile Phase A: H₂O (0.05% NH₄OH+10 mM ammonium bicarbonate): Mobile Phase B: ACN; gradient: of 42-62% of Mobile Phase B over 10 minutes; flow rate=30 mL/min) then lyophilized to afford E9 (34.1 mg, 33% yield) as a white solid. (LCMS) m/z (M+H)⁺=989.5; ¹H NMR (400 MHz, (CD₃)₂SO) δ ppm 11.10 (s, 1H), 8.50 (t, 1H), 8.22 (br s, 2H), 7.62 (dd, 1H), 7.31 (t, 1H), 7.14-7.01 (m, 3H), 6.99-6.83 (m, 3H), 5.38 (m, 1H), 4.78-4.63 (m, 3H), 4.37 (m, 2H), 3.81-3.72 (m, 2H), 3.67 (s, 5H), 3.62 (s, 3H), 3.54-3.43 (m, 1H), 3.07 (m, 2H), 2.97 (s, 6H), 2.92-2.82 (m, 1H), 2.78-2.57 (m, 4H), 2.24-2.06 (m, 2H), 2.02 (m, 1H), 1.87-1.68 (m, 4H), 1.50-1.29 (m, 16H).

### Example E10

### rac-(R)-5-(4-(Dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide (E10)

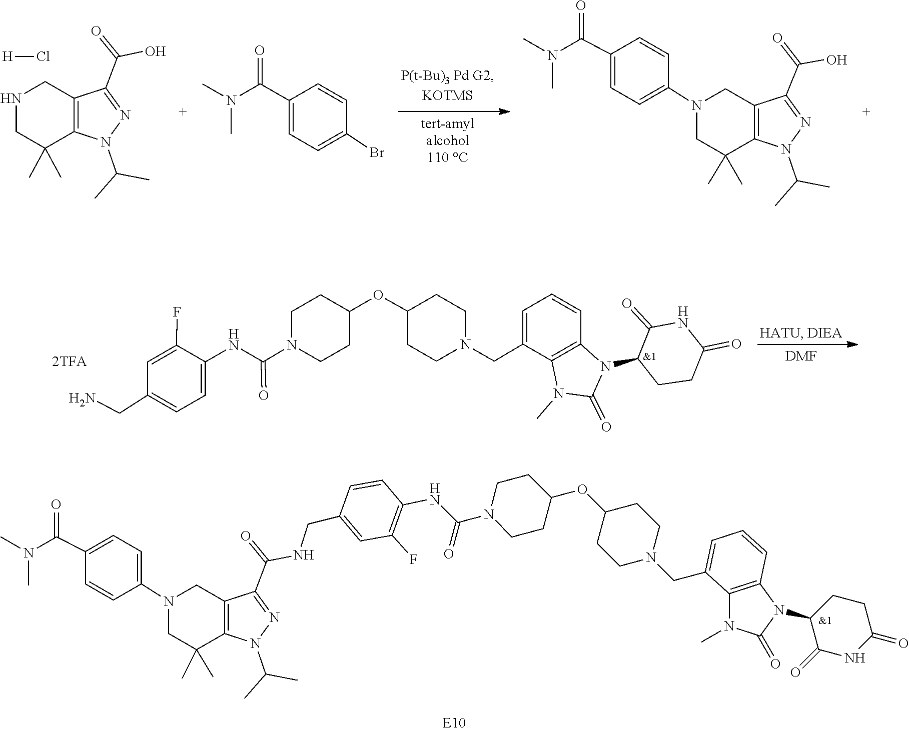

### Step 1. Preparation of 5-(4-(dimethylcarbamoyl)phenyl)-1-isopropyl-7,7-dimethyl-4,5,6,7-tetrahydro-1H-pyrazolo[4,3-c]pyridine-3-carboxylic acid

A mixture of 1-isopropyl-7,7-dimethyl-4,5,6,7-tetrahydro-1H-pyrazolo[4,3-c]pyridine-3-carboxylic acid hydrochloride (50 mg, 0.18 mmol) and 4-bromo-N,N-dimethylbenzamide (50 mg, 0.219 mmol) in tert-amyl alcohol (1 mL) was treated with KOTMS (82 mg, 0.639 mmol) and tris(tert-butyl)phosphine palladium G2 (4.68 mg, 0.00913 mmol) under an argon atmosphere. The reaction mixture was stirred at 110 °C for 1 h. After dilution with water (10 mL) and extraction with EtOAc (2×10 mL), the organic layer was discarded. The aqueous layer was adjusted to pH-3-4 using 1 M HCl and extracted with EtOAc (2×10 mL). The combined organic layers were dried over Na₂SO₄, filtered, and concentrated in vacuo to provide 5-(4-(dimethylcarbamoyl)phenyl)-1-isopropyl-7,7-dimethyl-4,5,6,7-tetrahydro-1H-pyrazolo[4,3-c]pyridine-3-carboxylic acid (54 mg, crude) as a yellow solid. The solid was used directly in the next step without further purification. (LCMS) m/z (M+H)+=385.1; ¹H NMR (400 MHz, (CD₃)₂SO) δ 12.93-12.32 (m, 1H), 7.35 (d, 2H), 6.98 (d, 2H), 4.78-4.70 (m, 1H), 4.27 (s, 2H), 3.40-3.36 (m, 2H), 2.97 (s, 6H), 1.43 (d, 6H), 1.40 (s, 6H).

### Step 2. Preparation of rac-(R)-5-(4-(dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide (E10)

A mixture of 5-(4-(dimethylcarbamoyl)phenyl)-1-isopropyl-7,7-dimethyl-4,5,6,7-tetrahydro-1H-pyrazolo[4,3-c]pyridine-3-carboxylic acid (54 mg, 0.14 mmol) and rac-(R)—N-(4-(aminomethyl)-2-fluorophenyl)-4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamide bis(2,2,2-trifluoroacetate) (119 mg, 0.14 mmol) in DMF (1 mL) was treated with HATU (54.5 mg, 0.143 mmol) and DIEA (81.7 mg, 0.632 mmol). The resulting yellow solution was stirred at room temperature for 2 hours. The reaction mixture was diluted with ACN (0.5 mL), filtered, and the filtrate was purified by reverse phase HPLC (Phenomenex Gemini-NX 150 mm×30 mm×5 μm column; Mobile Phase A: H₂O (0.05% NH₄OH+10 mM ammonium bicarbonate): Mobile Phase B: ACN, gradient: 37-77% of Mobile Phase B over 12 minutes; flow rate=30 mL/min) then lyophilized to afford E10 (37.4 mg, 27% yield) as a white solid. (LCMS) m/z (M+H)⁺=988.5. ¹H NMR (400 MHz, (CD₃)₂SO) δ 11.10 (s, 1H), 8.50 (m, 1H), 8.22 (s, 1H), 7.36-7.27 (m, 3H), 7.13-7.02 (m, 3H), 7.00-6.93 (m, 3H), 6.91-6.85 (m, 1H), 5.38 (m, 1H), 4.78-4.68 (m, 1H), 4.38 (m, 2H), 4.30 (s, 2H), 3.81-3.73 (m, 2H), 3.68 (s, 3H), 3.62 (br s, 3H), 3.49 (m, 1H), 3.40-3.34 (m, 2H), 3.08 (m, 2H), 2.96 (s, 6H), 2.91-2.81 (m, 1H), 2.79-2.57 (m, 4H), 2.23-2.09 (m, 2H), 2.06-1.97 (m, 1H), 1.77 (br s, 4H), 1.46 (m, 7H), 1.42-1.32 (m, 9H).

### Example E11

### (S)—N-(4-(3,3-Dimethylureido)-3-fluorobenzyl)-5-(4-(((1-((3-(2,4-dioxotetrahydropyrimidin-1(2H)-yl)-1-methyl-1H-indazol-7-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide (E11)

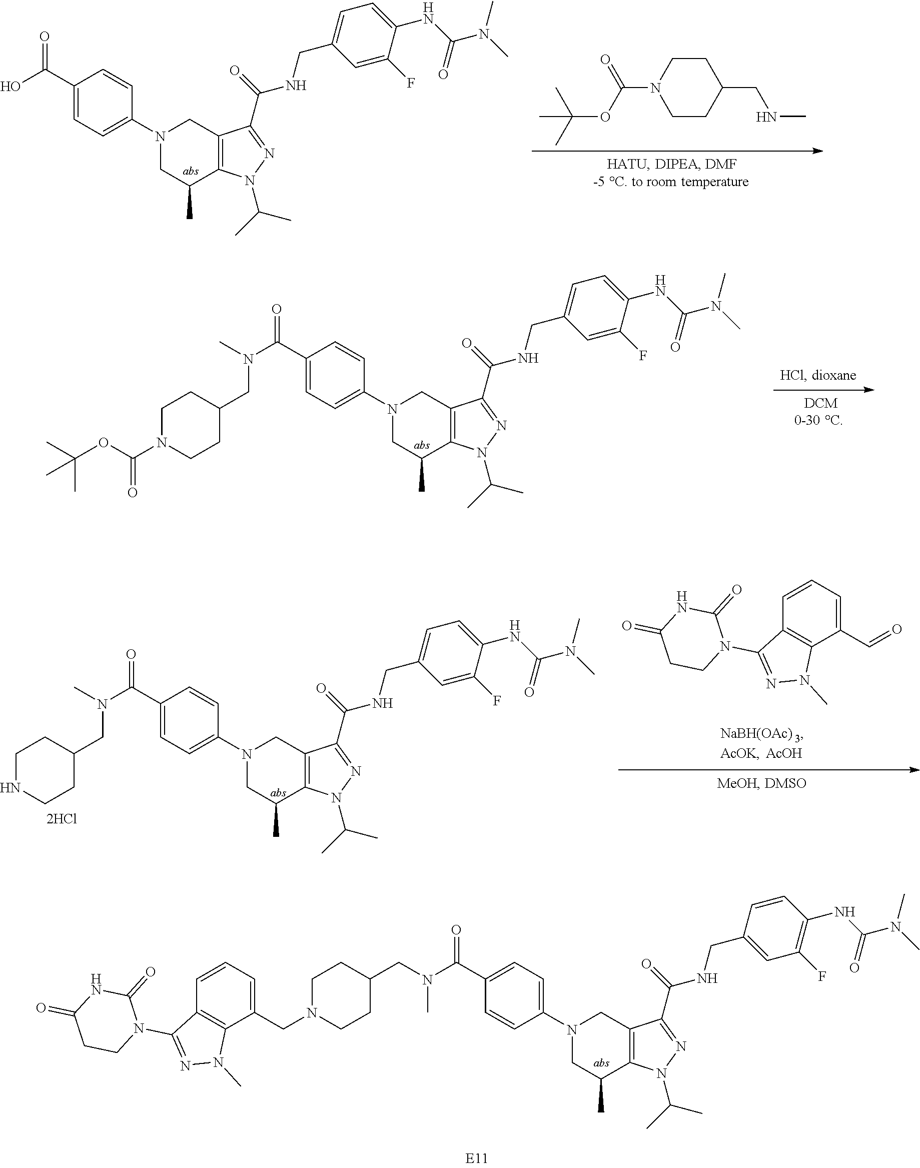

### Step 1. Preparation of tert-butyl (S)-4-((4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)-N-methylbenzamido)methyl)piperidine-1-carboxylate

At −5 °C, to a reaction mixture of (S)-4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)benzoic acid (0.12 g, 0.21 mmol) in DMF (2.0 mL) was added HATU (84 mg, 0.22 mmol) then stirred at −5 °C for 15 min before tert-butyl 4-((methylamino)methyl)piperidine-1-carboxylate (48 mg, 0.21 mmol) and DIEA (82 mg, 0.63 mmol) were added. The reaction mixture was slowly added to H₂O (15 mL) which caused precipitate to form. The suspension was filtered to provide tert-butyl (S)-4-((4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)-N-methylbenzamido)methyl)piperidine-1-carboxylate (0.15 g, crude) as a white solid. The solid was used directly in the next step without any further purification. (LCMS) m/z (M+H)+=747.4. ¹H NMR (400 MHz, (CD₃)₂SO) δ 8.51 (t, 1H), 7.97 (s, 1H), 7.39-7.24 (m, 3H), 7.14-7.01 (m, 2H), 6.96 (d, 2H), 4.58-4.49 (m, 2H), 4.38 (d, 2H), 4.10 (d, 1H), 3.99-3.77 (m, 3H), 3.32-3.19 (m, 2H), 2.97-2.88 (m, 9H), 1.89-1.85 (m, 1H), 1.58-1.54 (m, 2H), 1.48-1.35 (m, 15H), 1.32-1.22 (m, 6H), 0.97-0.92 (m, 3H).

### Step 2. Preparation of (S)—N-(4-(3,3-dimethylureido)-3-fluorobenzyl)-1-isopropyl-7-methyl-5-(4-(methyl(piperidin-4-ylmethyl)carbamoyl)phenyl)-4,5,6,7-tetrahydro-1H-pyrazolo[4,3-c]pyridine-3-carboxamide dihydrochloride

At 0-5 °C, to a solution of tert-butyl (S)-4-((4-(3-((4-(3,3-dimethylureido)-3-fluorobenzyl)carbamoyl)-1-isopropyl-7-methyl-1,4,6,7-tetrahydro-5H-pyrazolo[4,3-c]pyridin-5-yl)-N-methylbenzamido)methyl)piperidine-1-carboxylate (0.15 g, 0.18 mmol) in DCM (1 mL) was added HCl in dioxane (3 mL) then stirred at 30 °C for 2 h. The reaction mixture was concentrated in vacuo then lyophilized to provide (S)—N-(4-(3,3-dimethylureido)-3-fluorobenzyl)-1-isopropyl-7-methyl-5-(4-(methyl(piperidin-4-ylmethyl)carbamoyl)phenyl)-4,5,6,7-tetrahydro-1H-pyrazolo[4,3-c]pyridine-3-carboxamide dihydrochloride (0.14 g, crude) as a yellow solid. The solid was used directly in the next step without further purification. ¹H NMR (400 MHz, (CD₃)₂SO) δ 8.81-8.67 (m, 1H), 8.55-8.48 (m, 1H), 7.98 (s, 1H), 7.39-7.27 (m, 3H), 7.14-6.94 (m, 4H), 4.61-4.50 (m, 2H), 4.37 (d, 2H), 4.11 (d, 1H), 3.81 (d, 1H), 3.67-3.54 (m, 1H), 3.29 (dd, 6H), 2.95 (s, 3H), 2.90 (s, 6H), 2.86-2.79 (m, 2H), 2.01-1.97 (m, 1H), 1.76-1.71 (m, 2H), 1.44 (dd, 6H), 1.32-1.21 (m, 6H).

### Step 3. Preparation of (S)—N-(4-(3,3-dimethylureido)-3-fluorobenzyl)-5-(4-(((1-((3-(2,4-dioxotetrahydropyrimidin-1(2H)-yl)-1-methyl-1H-indazol-7-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide (E11)

To a reaction mixture of (S)—N-(4-(3,3-dimethylureido)-3-fluorobenzyl)-1-isopropyl-7-methyl-5-(4-(methyl(piperidin-4-ylmethyl)carbamoyl)phenyl)-4,5,6,7-tetrahydro-1H-pyrazolo[4,3-c]pyridine-3-carboxamide dihydrochloride (0.070 g, 0.092 mmol) and 3-(2,4-dioxotetrahydropyrimidin-1(2H)-yl)-1-methyl-1H-indazole-7-carbaldehyde (25 mg, 0.092 mmol) in DMSO (1 mL) and MeOH (0.1 mL) were added AcOK (18 mg, 0.18 mmol) and AcOH (11 mg, 0.18 mmol). The reaction mixture was stirred at room temperature for 1 h then NaBH(OAc)₃ (59 mg, 0.28 mmol) was added. The suspension was stirred at room temperature for 16 h then was quenched with 1 drop of H₂O, filtered and purified by reverse phase HPLC (Boston Prime C₁₈ 150 mm×30 mm×5 μm column; Mobile phase A: H₂O (0.05% NH₄OH); Mobile Phase B: ACN; gradient: 43-63% of Mobile Phase B over 10 min; flow rate: 35 mL/min) then lyophilized to provide E11 (0.010 g, 12% yield) as a white solid. (LCMS) m/z (M+H)⁺=904.5. ¹H NMR (400 MHz, (CD₃)₂SO) δ 10.55 (s, 1H), 8.51 (t, 1H), 7.98 (s, 1H), 7.54 (d, 1H), 7.35 (t, 1H), 7.30-7.23 (m, 2H), 7.19-6.90 (m, 6H), 4.60-4.49 (m, 2H), 4.37 (d, 2H), 4.26 (s, 3H), 4.10 (d, 1H), 3.87 (t, 2H), 3.84-3.70 (m, 3H), 3.33 (s, 2H), 3.30-3.17 (m, 4H), 2.93 (s, 3H), 2.90 (s, 6H), 2.85-2.69 (m, 4H), 2.00-1.96 (m, 2H), 1.78-1.51 (m, 3H), 1.43 (dd, 6H), 1.28 (d, 3H).

### Example E26

### 5-(5-(Dimethylcarbamoyl)pyridin-2-yl)-N-(4-(4-((6-(2-(2,6-dioxopiperidin-3-yl)-3-oxoisoindolin-5-yl)hex-5-yn-1-yl)oxy)piperidine-1-carboxamido)benzyl)-1-isopropyl-4,5,6,7-tetrahydro-1H-pyrazolo[4,3-c]pyridine-3-carboxamide bis(2,2,2-trifluoroacetate) (E26)

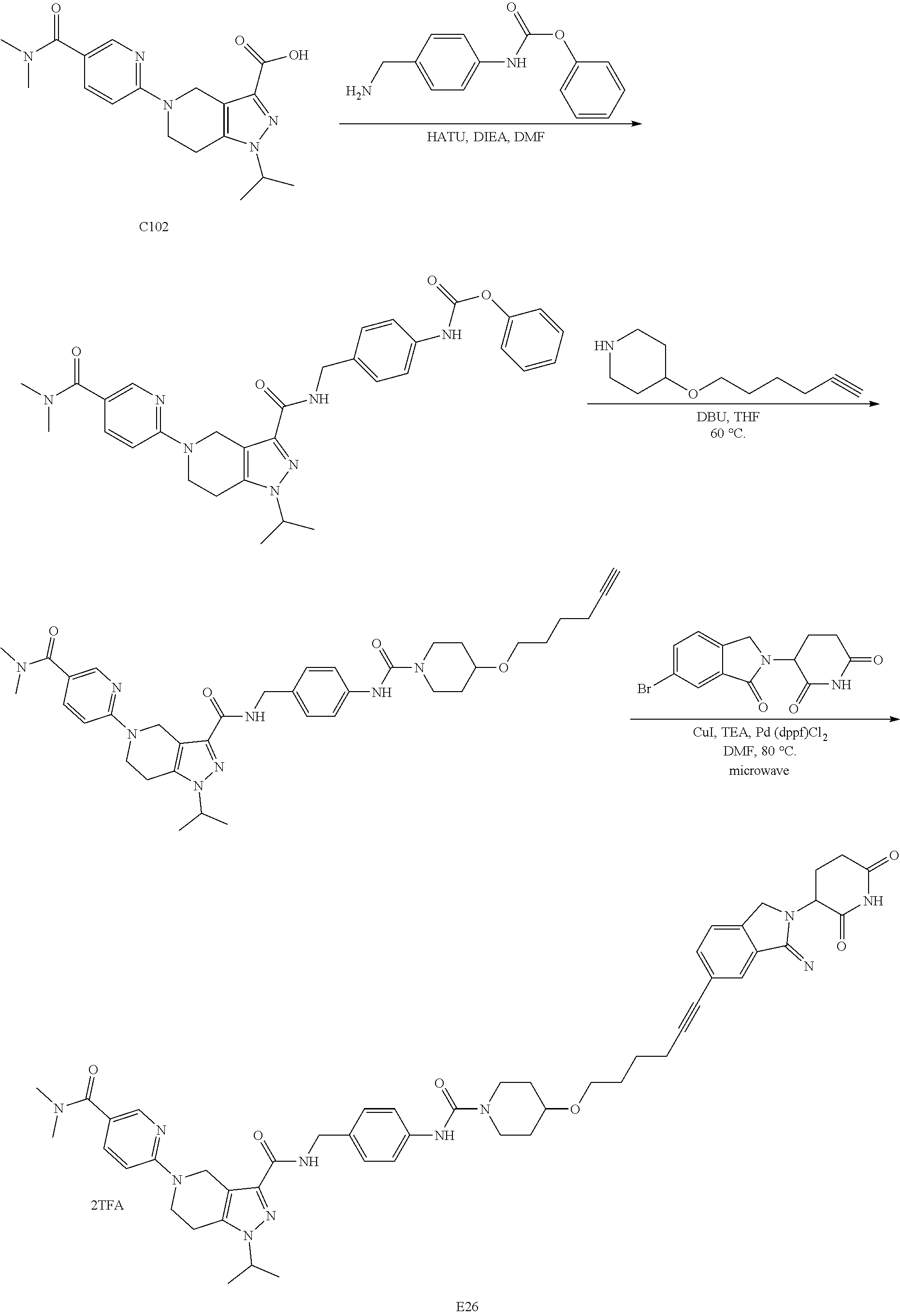

### Step 1. Preparation of phenyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate

To a reaction mixture of C102 (3.16 g, 8.83 mmol) in DMF (40 mL) was added HATU (3.36 g, 8.83 mmol). The reaction mixture was stirred at room temperature for 10 min then phenyl (4-(aminomethyl)phenyl)carbamate (CAS: 1019455-66-3; 3.31 g, 11.9 mmol) and DIEA (4.56 g, 35.3 mmol) were added. The reaction was stirred at room temperature for 16 h then poured into H₂O and extracted with (10:1, DCM: MeOH)(100 mL×3). The combined organic phase was washed with 1 M Na₂CO₃ (20 mL) then brine (15 mL×2). The organic phase was concentrated in vacuo to give the residue which was purified by column chromatography (silica gel; 0-10% MeOH: DCM). The yellow gum was triturated with MTBE (20 mL) and stirred at room temperature for 16 h, which caused solid to precipitate. The suspension was filtered, and the filter cake was collected to provide phenyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (1.34 g, 19.4% yield) as a white solid. (LC/MS) m/z (M+H)+=582.3. ¹H NMR (400 MHz, (CD₃)₂SO) δ 10.19 (s, 1H), 8.41 (t, 1H), 8.24 (d, 1H), 7.63 (dd, 1H), 7.48-7.33 (m, 4H), 7.31-7.18 (m, 5H), 6.88 (d, 1H), 4.74 (s, 2H), 4.55-4.44 (m, 1H), 4.37 (d, 2H), 3.95 (t, 2H), 2.96 (s, 6H), 2.84-2.80 (m, 2H), 1.39 (d, 6H).

### Step 2. Preparation of 5-(5-(dimethylcarbamoyl)pyridin-2-yl)-N-(4-(4-(hex-5-yn-1-yloxy)piperidine-1-carboxamido)benzyl)-1-isopropyl-4,5,6,7-tetrahydro-1H-pyrazolo[4,3-c]pyridine-3-carboxamide

To a reaction mixture of the phenyl (4-((5-(5-(dimethylcarbamoyl)pyridin-2-yl)-1-isopropyl-4,5,6,7-tetrahydro-1H-pyrazolo[4,3-c]pyridine-3-carboxamido)methyl)phenyl)carbamate (145 mg, 0.250 mmol) and 4-(hex-5-yn-1-yloxy)piperidine (CAS: 2139819-04-6; 72.5 mg, 0.400 mmol) in THF (2.0 mL) was added DBU (202 mg, 1.33 mmol). The reaction mixture was stirred at 60 °C for 1 h then concentrated in vacuo. The residue was diluted with DCM then washed with aqueous NH₄Cl. The organic layer was separated then concentrated in vacuo to provide 5-(5-(dimethylcarbamoyl)pyridin-2-yl)-N-(4-(4-(hex-5-yn-1-yloxy)piperidine-1-carboxamido)benzyl)-1-isopropyl-4,5,6,7-tetrahydro-1H-pyrazolo[4,3-c]pyridine-3-carboxamide (0.167 g, crude). The crude was used directly in the next step without further purification. (LC/MS) m/z (M+H)⁺=669.9.

### Step 3. Preparation of 5-(5-(dimethylcarbamoyl)pyridin-2-yl)-N-(4-(4-((6-(2-(2,6-dioxopiperidin-3-yl)-3-oxoisoindolin-5-yl)hex-5-yn-1-yl)oxy)piperidine-1-carboxamido)benzyl)-1-isopropyl-4,5,6,7-tetrahydro-1H-pyrazolo[4,3-c]pyridine-3-carboxamide bis(2,2,2-trifluoroacetate) (E26)

To a reaction mixture of 3-(6-bromo-1-oxoisoindolin-2-yl)piperidine-2,6-dione (CAS: 2304513-76-4; 8.0 mg, 0.10 mmol), Pd(dppf)Cl₂ (4.1 mg, 5.0 μmol) and CuI (1.0 mg, 5.0 μmol) was added 5-(5-(dimethylcarbamoyl)pyridin-2-yl)-N-(4-(4-(hex-5-yn-1-yloxy)piperidine-1-carboxamido)benzyl)-1-isopropyl-4,5,6,7-tetrahydro-1H-pyrazolo[4,3-c]pyridine-3-carboxamide (1.3 mg, 0.050 mmol) in DMF (0.250 mL) followed by TEA (0.36 g, 3.6 mmol). The reaction mixture was degassed with nitrogen gas for 1 min then stirred at 80 °C for 30 minutes via microwave. The suspension was diluted in DCM (3 mL) then concentrated in vacuo. The residue was redissolved in DCM (3 mL) then washed with aqueous NH₄Cl (2 mL). The organic layer was separated and concentrated in vacuo. The residue was dissolved in DMSO (1 mL) and filtered. The solution was purified by reverse phase HPLC (Sunfire C₁₈ 100 mm×19 mm×5 μm column; Mobile Phase A: H2O (0.05% TFA): Mobile Phase B: ACN (0.05% TFA); gradient: 20%-95% of Mobile Phase B over 9.0 min then held at 95% of Mobile Phase B for 1.0 min; flow rate: 25 mL/min) to provide E26 (5.9 mg, 13% yield). (LC/MS) m/z (M+H)⁺=910.4.

### Example E91

### (S)-5-(4-(Dimethylcarbamoyl)phenyl)-N-(4-(2-((1-((1-((RS)-2,6-dioxopiperidin-3-yl)-3-methyl-2-oxo-2,3-dihydro-1H-benzo[d]imidazol-5-yl)methyl)piperidin-4-yl)oxy)acetamido)benzyl)-1-isopropyl-7-methyl-4,5,6,7-tetrahydro-1H-pyrazolo[4,3-c]pyridine-3-carboxamide (E91)

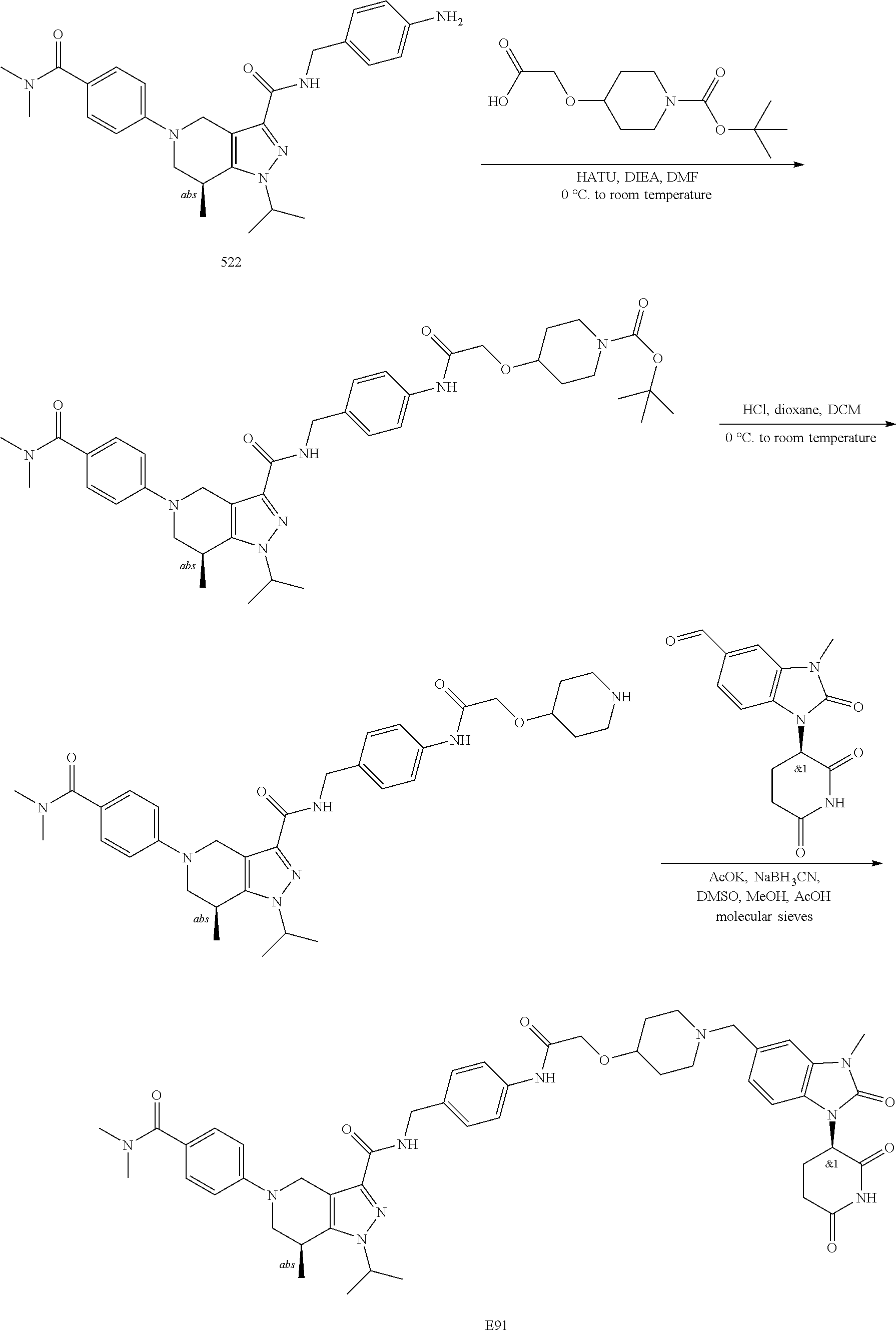

### Step 1. Preparation of tert-butyl (S)-4-(2-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)phenyl)amino)-2-oxoethoxy)piperidine-1-carboxylate

At 0 °C, to the mixture of 2-((1-(tert-butoxycarbonyl)piperidin-4-yl)oxy)acetic acid (CAS: 157688-46-5; 49 mg, 0.19 mmol) in DMF (1 mL) was added HATU (71 mg, 0.19 mmol). The reaction mixture was stirred at 0 °C for 10 min then 522 (85 mg, 0.18 mmol) and DIEA (93 mg, 0.72 mmol) were added. The reaction mixture was stirred at room temperature for 16 h then poured into H₂O and extracted with (10:1, DCM: MeOH)(10 mL×3). The organic phase was washed with brine (15 mL×2) then concentrated in vacuo to provide tert-butyl (S)-4-(2-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)phenyl)amino)-2-oxoethoxy)piperidine-1-carboxylate (130 mg, crude) as a yellow gum. The gum was used directly in the next step without further purification. (LC/MS) m/z (M+H)⁺=716.4.

### Step 2. Preparation of (S)-5-(4-(dimethylcarbamoyl)phenyl)-1-isopropyl-7-methyl-N-(4-(2-(piperidin-4-yloxy)acetamido)benzyl)-4,5,6,7-tetrahydro-1H-pyrazolo[4,3-c]pyridine-3-carboxamide hydrochloride

At 0 °C, to a solution of tert-butyl (S)-4-(2-((4-((5-(4-(dimethylcarbamoyl)phenyl)-1-isopropyl-7-methyl-4,5,6,7-tetrahydro-1H-pyrazolo[4,3-c]pyridine-3-carboxamido)methyl)phenyl)amino)-2-oxoethoxy)piperidine-1-carboxylate (0.13 g, 0.13 mmol) in DCM (5 mL) was added HCl in dioxane (20 mL). The reaction mixture was stirred at room temperature for 2 h then concentrated in vacuo and lyophilized to provide (S)-5-(4-(dimethylcarbamoyl)phenyl)-1-isopropyl-7-methyl-N-(4-(2-(piperidin-4-yloxy)acetamido)benzyl)-4,5,6,7-tetrahydro-1H-pyrazolo[4,3-c]pyridine-3-carboxamide hydrochloride (95 mg, crude) as a yellow solid. The solid was used directly in the next step without further purification. (LC/MS) m/z (M+H)⁺=616.4.

### Step 3. Preparation of (S)-5-(4-(dimethylcarbamoyl)phenyl)-N-(4-(2-((1-((1-((RS)-2,6-dioxopiperidin-3-yl)-3-methyl-2-oxo-2,3-dihydro-1H-benzo[d]imidazol-5-yl)methyl)piperidin-4-yl)oxy)acetamido)benzyl)-1-isopropyl-7-methyl-4,5,6,7-tetrahydro-1H-pyrazolo[4,3-c]pyridine-3-carboxamide (E91)

To a solution of (S)-5-(4-(dimethylcarbamoyl)phenyl)-1-isopropyl-7-methyl-N-(4-(2-(piperidin-4-yloxy)acetamido)benzyl)-4,5,6,7-tetrahydro-1H-pyrazolo[4,3-c]pyridine-3-carboxamide hydrochloride (95 mg, 0.15 mmol) and rac-(R)-1-(2,6-dioxopiperidin-3-yl)-3-methyl-2-oxo-2,3-dihydro-1H-benzo[d]imidazole-5-carbaldehyde (CAS: 2357116-00-6; 53 mg, 0.18 mmol) in DMSO (1.0 mL) and MeOH (0.2 mL) was added AcOK (0.030 g, 0.31 mmol), AcOH (9.3 mg, 0.15 mmol), powdered molecular sieves (0.050 g) and NaBH₃CN (29 mg, 0.46 mmol). The reaction mixture was degassed with nitrogen gas for 2 minutes then stirred at room temperature for 18 h. The suspension poured into H₂O (10 mL) and extracted with (10:1, DCM: MeOH)(10 mL×3). The organic layer was concentrated in vacuo then diluted with DMF. The solution was purified by reverse phase HPLC (Welch Xtimate C₁₈ 150 mm×30 mm×5 μm column; Mobile phase A: H₂O (0.225% formic acid); Mobile Phase B: ACN; gradient: 10-50% of Mobile Phase B over 11 min; flow rate: 25 mL/min) and lyophilized to provide E91 (0.020 g, 15% yield) as a white solid. (LC/MS) m/z (M+H)⁺=887.6. ¹H NMR (400 MHz, (CD₃)₂SO) δ 11.09 (s, 1H), 9.54 (s, 1H), 8.41 (t, 1H), 7.57 (d, 2H), 7.33 (d, 2H), 7.26 (d, 2H), 7.09 (s, 1H), 7.07-7.00 (m, 1H), 7.00-6.92 (m, 3H), 5.35 (dd, 1H), 4.60-4.50 (m, 2H), 4.37 (d, 2H), 4.14-4.00 (m, 3H), 3.82 (d, 1H), 3.49-3.34 (m, 5H), 3.31-3.16 (m, 3H), 2.96 (s, 6H), 2.92-2.84 (m, 1H), 2.77-2.57 (m, 4H), 2.14-1.96 (m, 3H), 1.89-1.84 (m, 2H), 1.58-1.50 (m, 2H), 1.43 (dd, 6H), 1.28 (d, 3H).

### Example E215

### (S)-5-(5-((2-(1-(3-(2,4-Dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (E215)

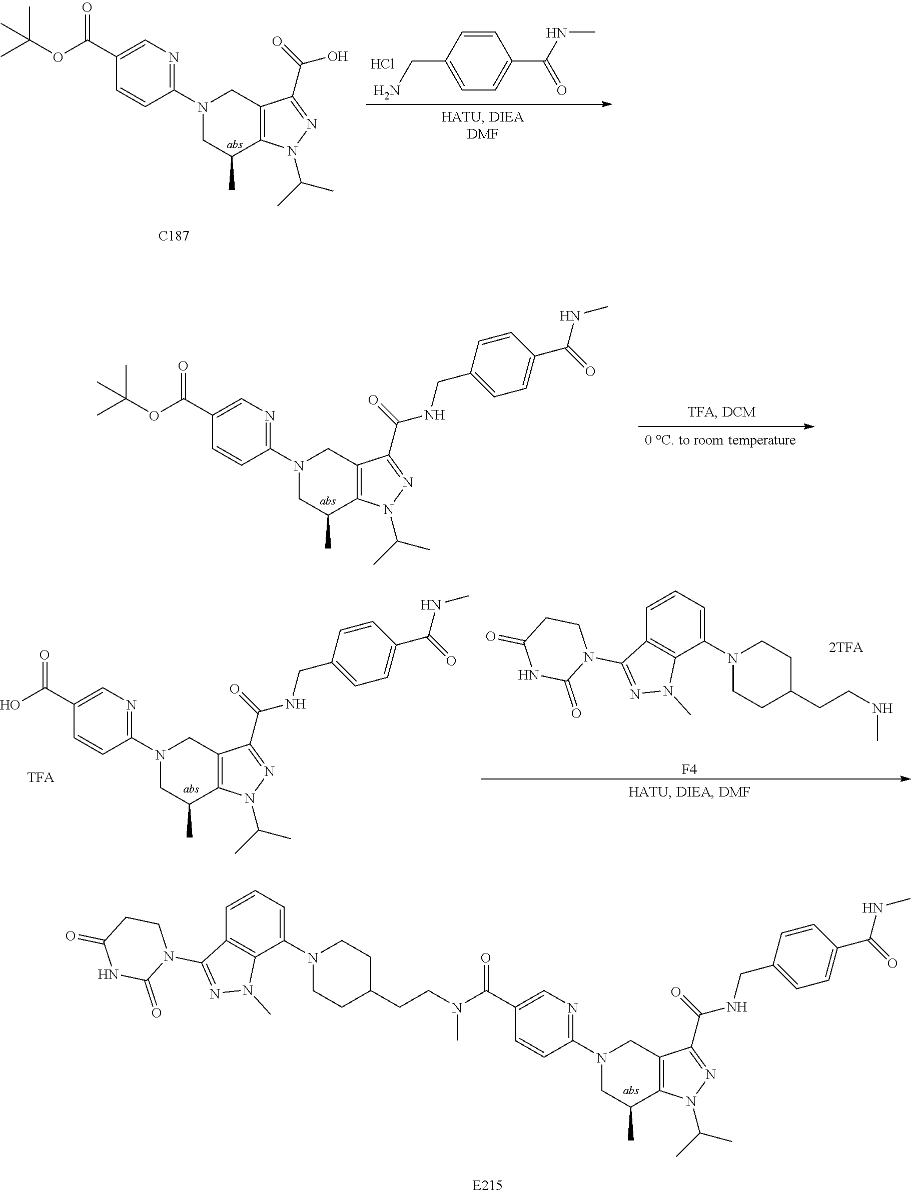

### Step 1. Preparation of tert-butyl (S)-6-(1-isopropyl-7-methyl-3-((4-(methylcarbamoyl)benzyl)carbamoyl)-1,4,6,7-tetrahydro-5H-pyrazolo[4,3-c]pyridin-5-yl)nicotinate

To a solution of C187 (1.00 g, 2.50 mmol) and 4-(aminomethyl)-N-methylbenzamide hydrochloride (CAS: 1158467-80-1; 0.592 g, 2.50 mmol) in DMF (10 mL) were added HATU (0.968 g, 2.55 mmol) and DIEA (1.13 g, 8.74 mmol). The reaction mixture was stirred at room temperature for 16 h then poured into H₂O (50 mL) and filtered. The filtered cake was concentrated in vacuo and lyophilized to provide tert-butyl (S)-6-(1-isopropyl-7-methyl-3-((4-(methylcarbamoyl)benzyl)carbamoyl)-1,4,6,7-tetrahydro-5H-pyrazolo[4,3-c]pyridin-5-yl)nicotinate (1.06 g, crude) as a white solid. The solid was used directly in the next step without further purification. (LC/MS) m/z (M+H)⁺=547.4. ¹H NMR (400 MHz, CDCl₃) δ 8.72-8.66 (m, 1H), 8.05-7.97 (m, 1H), 7.75-7.69 (m, 2H), 7.41-7.31 (m, 3H), 6.79 (d, 1H), 6.49-6.40 (m, 1H), 5.02 (d, 1H), 4.80 (d, 1H), 4.69-4.54 (m, 2H), 4.52-4.35 (m, 2H), 3.45-3.34 (m, 1H), 3.16-3.06 (m, 1H), 2.98 (d, 3H), 1.55 (s, 9H), 1.52-1.35 (m, 6H), 1.26 (d, 3H).

### Step 2. Preparation of (S)-6-(1-isopropyl-7-methyl-3-((4-(methylcarbamoyl)benzyl)carbamoyl)-1,4,6,7-tetrahydro-5H-pyrazolo[4,3-c]pyridin-5-yl)nicotinic acid trifluoroacetate

At 0 °C, to a solution of tert-butyl (S)-6-(1-isopropyl-7-methyl-3-((4-(methylcarbamoyl)benzyl)carbamoyl)-1,4,6,7-tetrahydro-5H-pyrazolo[4,3-c]pyridin-5-yl)nicotinate (2.40 g, 4.39 mmol) in DCM (20 mL) was added TFA (5 mL). The reaction mixture was stirred at room temperature for 16 h then concentrated in vacuo. The residue was lyophilized to provide (S)-6-(1-isopropyl-7-methyl-3-((4-(methylcarbamoyl)benzyl)carbamoyl)-1,4,6,7-tetrahydro-5H-pyrazolo[4,3-c]pyridin-5-yl)nicotinic acid trifluoroacetate (2.65 g, crude) as a white solid. The solid was used directly in the next step without further purification. (LC/MS) m/z (M+H)⁺=491.3. ¹H NMR (400 MHz, (CD₃)₂SO) δ 8.63-8.55 (m, 2H), 8.37 (q, 1H), 8.00 (dd, 1H), 7.82-7.75 (m, 2H), 7.41-7.35 (m, 2H), 7.03 (d, 1H), 5.17 (d, 1H), 4.60-4.24 (m, 4H), 3.42 (dd, 1H), 3.33-3.23 (m, 1H), 2.77 (d, 3H), 1.48-1.35 (m, 6H), 1.30-1.21 (m, 1H), 1.18 (d, 3H).

### Step 3. Preparation of (S)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide- (E215)

To a solution of 1-(1-methyl-7-(4-(2-(methylamino)ethyl)piperidin-1-yl)-1H-indazol-3-yl)dihydropyrimidine-2,4(1H,3H)-dione trifluoroacetate (0.150 g, 0.301 mmol) and (S)-6-(1-isopropyl-7-methyl-3-((4-(methylcarbamoyl)benzyl)carbamoyl)-1,4,6,7-tetrahydro-5H-pyrazolo[4,3-c]pyridin-5-yl)nicotinic acid trifluoroacetate (182 mg, 0.301 mmol) in DMF (3 mL) were added HATU (0.120 g, 0.316 mmol) and DIEA (311 mg, 2.41 mmol). The reaction mixture was stirred at room temperature for 1 h then poured into H₂O (10 mL) and filtered. The filtered cake was dissolved in DMF (1 mL). The solution was purified by reverse phase HPLC (Boston Prime C₁₈ 200 mm×40 mm×10 μm column; Mobile phase A: H₂O (0.225% formic acid)/Mobile Phase B: ACN; gradient: 38-58% of Mobile Phase B over 15 min; flow rate: 60 mL/min) then lyophilized to provide E215 (84.8 mg, 32.7% yield) as a white solid. (LC/MS) m/z (M+H)⁺=858.5. ¹H NMR (600 MHz, CD₃CN at 328 K) δ 8.26-8.23 (m, 2H), 7.73 (d, 2H), 7.64 (t, 1H), 7.60 (dd, 1H), 7.41 (d, 2H), 7.32-7.26 (m, 1H), 7.05-7.01 (m, 2H), 7.00 (s, 0.09H), 6.89-6.80 (m, 2H), 5.08 (d, 1H), 4.63-4.44 (m, 4H), 4.33 (d, 1H), 4.24 (s, 3H), 3.93 (t, 2H), 3.50 (t, 2H), 3.38 (dd, 1H), 3.24-3.15 (m, 3H), 3.02 (s, 3H), 2.86 (dd, 3H), 2.78 (t, 2H), 2.72-2.68 (m, 2H), 1.78-1.74 (m, 2H), 1.65 (q, 2H), 1.55-1.45 (m, 9H), 1.25 (d, 3H).

### Example E230

### 5-(5-((2-(4-(3-(2,4-Dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide (E230)

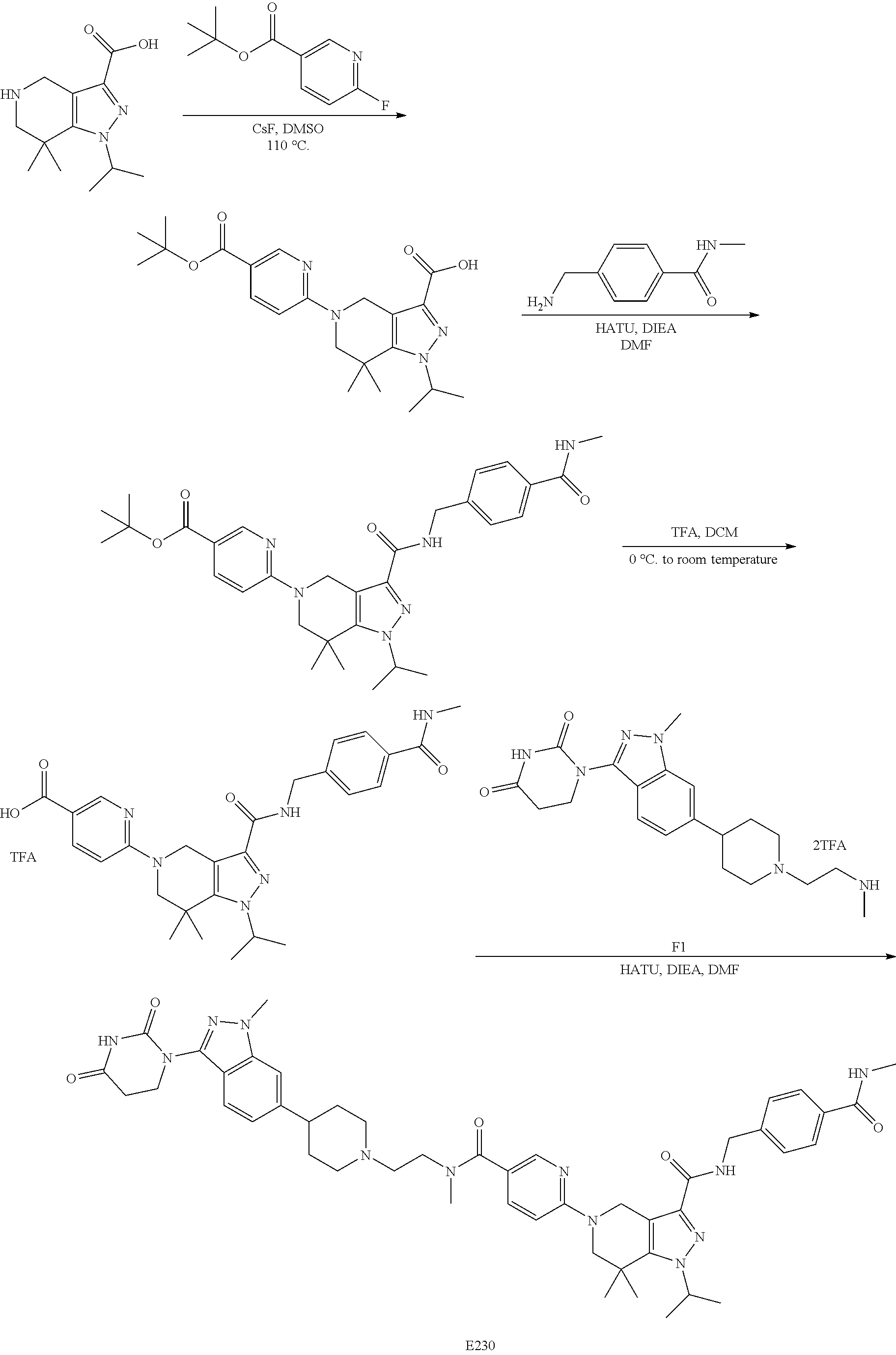

### Step 1. Preparation of 5-(5-(tert-butoxycarbonyl)pyridin-2-yl)-1-isopropyl-7,7-dimethyl-4,5,6,7-tetrahydro-1H-pyrazolo[4,3-c]pyridine-3-carboxylic acid

To a solution of 1-isopropyl-7,7-dimethyl-4,5,6,7-tetrahydro-1H-pyrazolo[4,3-c]pyridine-3-carboxylic acid (2.50 g, 9.13 mmol) and tert-butyl 6-fluoronicotinate (CAS: 676560-01-3, 1.98 g, 10.0 mmol) in DMSO (25 mL) was added CsF (4.16 g, 27.4 mmol) under argon atmosphere. The reaction mixture was stirred at 110° under argon gas for 16 h then poured into water (150 mL). The suspension was extracted with EtOAc (30 mL×5). The combined organic layers were dried over Na₂SO₄, filtered then concentrated in vacuo. The residue was triturated with (3:1, EtOAc: n-hexane) (40 mL) to give 5-(5-(tert-butoxycarbonyl)pyridin-2-yl)-1-isopropyl-7,7-dimethyl-4,5,6,7-tetrahydro-1H-pyrazolo[4,3-c]pyridine-3-carboxylic acid (2.55 g, 67.4% yield) as a white solid. (LC/MS) m/z (M+H)⁺=415.3. ¹H NMR (400 MHz, (CD₃)₂SO) δ 12.72 (s, 1H), 8.61 (d, 1H), 7.90 (dd, 1H), 6.95 (d, 1H), 4.72 (d, 3H), 3.73 (s, 2H), 1.51 (s, 9H), 1.40 (d, 6H), 1.34 (s, 6H).

### Step 2. Preparation of tert-butyl 6-(1-isopropyl-7,7-dimethyl-3-((4-(methylcarbamoyl)benzyl)carbamoyl)-1,4,6,7-tetrahydro-5H-pyrazolo[4,3-c]pyridin-5-yl)nicotinate

To a mixture of 5-(5-(tert-butoxycarbonyl)pyridin-2-yl)-1-isopropyl-7,7-dimethyl-4,5,6,7-tetrahydro-1H-pyrazolo[4,3-c]pyridine-3-carboxylic acid (1.00 g, 2.41 mmol) and 4-(aminomethyl)-N-methylbenzamide (0.484 g, 2.41 mmol) in DMF (10 mL) was added HATU (0.963 g, 2.53 mmol) then DIEA (0.935 g, 7.24 mmol). The reaction mixture was stirred at room temperature for 16 h then slowly added to ice water (40 mL) which caused some precipitate to form, the solid was filtered, collected and lyophilized over 16 h to provide tert-butyl 6-(1-isopropyl-7,7-dimethyl-3-((4-(methylcarbamoyl)benzyl)carbamoyl)-1,4,6,7-tetrahydro-5H-pyrazolo[4,3-c]pyridin-5-yl)nicotinate (1.25 g, 92.4% yield) as a white solid. (LC/MS) m/z (M+H)⁺=561.4.

### Step 3. Preparation of 6-(1-isopropyl-7,7-dimethyl-3-((4-(methylcarbamoyl)benzyl)carbamoyl)-1,4,6,7-tetrahydro-5H-pyrazolo[4,3-c]pyridin-5-yl)nicotinic acid trifluoroacetate

At 0 °C, to a solution of tert-butyl 6-(1-isopropyl-7,7-dimethyl-3-((4-(methylcarbamoyl)benzyl)carbamoyl)-1,4,6,7-tetrahydro-5H-pyrazolo[4,3-c]pyridin-5-yl)nicotinate (0.200 g, 0.357 mmol) in DCM (2 mL) was added TFA (2 mL). The reaction mixture was stirred at room temperature for 2 h then concentrated in vacuo to provide 6-(1-isopropyl-7,7-dimethyl-3-((4-(methylcarbamoyl)benzyl)carbamoyl)-1,4,6,7-tetrahydro-5H-pyrazolo[4,3-c]pyridin-5-yl)nicotinic acid trifluoroacetate (0.180 g, crude) as a yellow solid. The solid was used directly in the next step without further purification. (LC/MS) m/z (M+H)⁺=505.3.

### Step 4. Preparation of 5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide (E230)

To a reaction mixture of 6-(1-isopropyl-7,7-dimethyl-3-((4-(methylcarbamoyl)benzyl)carbamoyl)-1,4,6,7-tetrahydro-5H-pyrazolo[4,3-c]pyridin-5-yl)nicotinic acid trifluoroacetate (0.060 g, 0.073 mmol) and 1-(1-methyl-6-(1-(2-(methylamino)ethyl)piperidin-4-yl)-1H-indazol-3-yl)dihydropyrimidine-2,4(1H,3H)-dione bis(2,2,2-trifluoroacetate) (45 mg, 0.073 mmol) in DMF (1 mL) was added HATU (28 mg, 0.074 mmol) and DIEA (38 mg, 0.29 mmol). The yellow solution was stirred for 2 h then diluted with ACN. The solution was purified by reverse phase HPLC (WePure Biotech XP tC₁₈ 150 mm×40 mm×7 μm, Mobile phase A: H₂O (0.05% NH₃H₂O+10 mM NH₄HCO₃)/Mobile Phase B: ACN; gradient: Mobile Phase B from 28-68% over 12 min; flow rate: 60 mL/min) and lyophilized to provide E230 (23 mg, 36% yield) as a white solid. (LC/MS) m/z (M+H)+=871.5. ¹H NMR (400 MHz, (CD₃)₂SO) δ 10.54 (s, 1H), 8.54 (t, 1H), 8.39-8.34 (m, 1H), 8.25-8.20 (m, 1H), 7.77 (d, 2H), 7.67-7.60 (m, 1H), 7.54 (d, 1H), 7.45-7.34 (m, 3H), 7.02 (d, 1H), 6.92 (d, 1H), 4.70-4.65 (m, 3H), 4.45 (d, 2H), 3.96 (s, 3H), 3.90 (t, 2H), 3.67 (s, 2H), 3.52-3.48 (m, 1H), 3.40-3.33 (m, 1H), 3.32-3.25 (m, 4H), 3.01 (s, 4H), 2.79-2.70 (m, 5H), 2.08-2.04 (m, 2H), 1.76-1.72 (m, 4H), 1.43 (d, 6H), 1.33 (s, 6H).

### Example E248

### (S)-5-(5-((2-(4-(3-(2,4-Dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (E248)

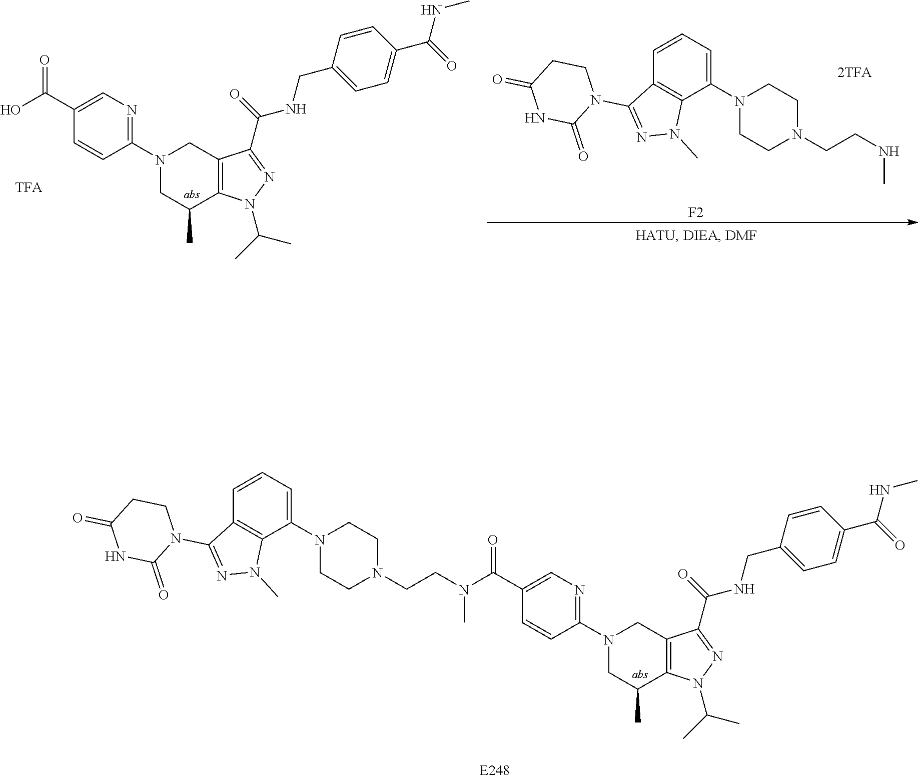

### Step 1. Preparation of (S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (E248)

To a solution of 1-(1-methyl-7-(4-(2-(methylamino)ethyl)piperazin-1-yl)-1H-indazol-3-yl)dihydropyrimidine-2,4(1H,3H)-dione bis(2,2,2-trifluoroacetate) (0.150 g, 0.245 mmol) and (S)-6-(1-isopropyl-7-methyl-3-((4-(methylcarbamoyl)benzyl)carbamoyl)-1,4,6,7-tetrahydro-5H-pyrazolo[4,3-c]pyridin-5-yl)nicotinic acid trifluoroacetate (0.148 g, 0.245 mmol) in DMF (4 mL) were added HATU (94.8 mg, 0.249 mmol) and DIEA (221 mg, 1.71 mmol). The mixture was stirred at room temperature for 1 h then poured into water (25 mL) and filtered. The filter cake was diluted with DMSO and ACN then purified by reverse phase HPLC (Pure Biotech XP tC₁₈ 150 mm×40 mm×7 μm column; Mobile phase A: H₂O (0.225% formic acid)/Mobile Phase B: ACN; gradient: 12-32% of Mobile Phase B over 10 min; flow rate: 60 mL/min) then lyophilized to provide E248 (93.9 mg, 44.4% yield) as a white solid. (LC/MS) m/z (M+H)⁺=858.5. ¹H NMR (400 MHz, (CD₃)₂SO) δ 10.54 (s, 1H), 8.55 (t, 1H), 8.37 (q, 1H), 8.24 (d, 1H), 7.81-7.74 (m, 2H), 7.64 (dd, 1H), 7.38 (d, 2H), 7.30 (dd, 1H), 7.07-6.97 (m, 2H), 6.91 (d, 1H), 5.11 (d, 1H), 4.57-4.40 (m, 4H), 4.27-4.19 (m, 4H), 3.88 (t, 2H), 3.56-3.51 (m, 2H), 3.29-2.80 (m, 10H), 2.79-2.71 (m, 6H), 2.69-2.53 (m, 2H), 2.35-2.30 (m, 2H), 1.42 (dd, 6H), 1.18 (d, 3H).

### Example E273

### (S)—N-(4-(3,3-Dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide (E273)

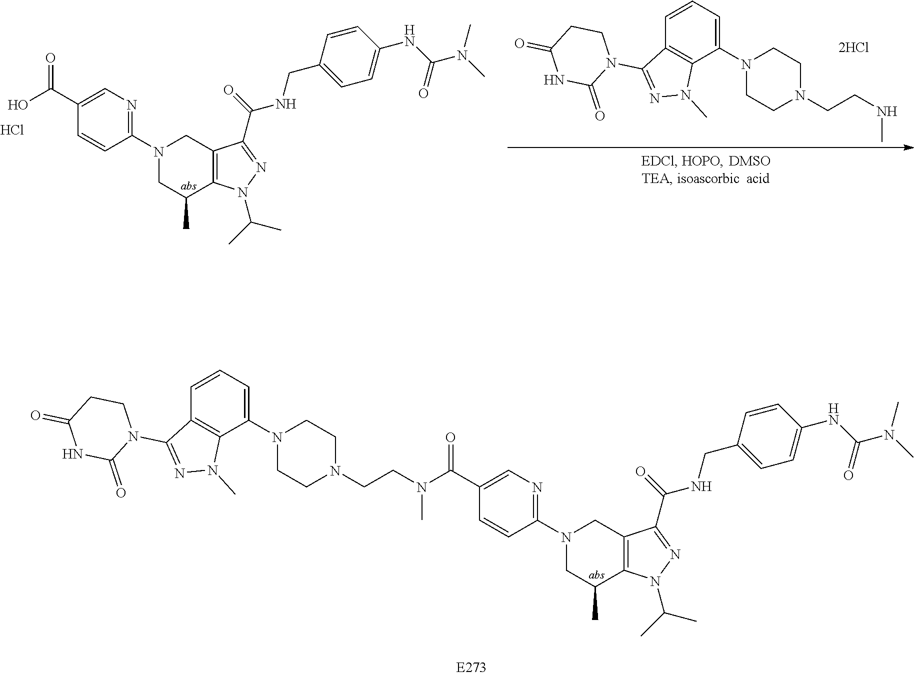

### Step 1. Preparation of (S)—N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide (E273)

In a 50 mL round bottom flask was added (S)-6-(3-((4-(3,3-dimethylureido)benzyl)carbamoyl)-1-isopropyl-7-methyl-1,4,6,7-tetrahydro-5H-pyrazoo[4,3-c]pyridin-5-yl)nicotinic acid hydrochloride (F74, 1.00 g, 1.85 mmol), isoascorbic acid (16.3 mg, 9.9 μL, 92.4 μmol), DMSO (7 mL) and TEA (18.7 mg, 25.8 μL, 0.185 mmol). To the solution was added HOPO (236 mg, 212.5 μL, 2.12 mmol) then EDCI (443 mg, 2.31 mmol). The reaction mixture was stirred at room temperature overnight before TEA (934 mg, 1.3 mL, 9.24 mmol) and 1-(1-methyl-7-(4-(2-(methylamino)ethyl)piperazin-1-yl)-1H-indazol-3-yl)dihydropyrimidine-2,4(1H,3H)-dione dihydrochloride (F2A, 955 mg, 1.96 mmol) were added. The slurry was stirred at room temperature for 3 h. The pH was adjusted to pH=7 with the addition of 1 M HCl (2.0 mL) followed by 0.25% sodium ascorbate in pH=7 buffer [0.005M of KH₂PO₄/Na₂HPO₄](20 mL). The slurry was stirred at room temperature for 3.5 h then filtered through a disposable funnel. The filter cake was rinsed with pH=7 buffer [0.005M of KH₂PO₄/Na₂HPO₄](5×7 mL) then collected and dried by vacuum under nitrogen atmosphere overnight to give (S)—N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide (1.72 g, crude) as an off-white solid. The solid was triturated in 0.25% sodium ascorbate in acetone: pH=7 buffer [0.005 M KH₂PO₄/Na₂HPO₄](3:97 volume/volume, 17 mL) at room temperature for 4 h then filtered through a disposable filter. The filter cake was rinsed with pH=7 buffer [0.005 M of KH₂PO₄/Na₂HPO₄](4×10 mL) then water (10 mL). The filter cake was collected and dried by vacuum under nitrogen atmosphere overnight to provide E273 (1.66 g, 99.7% yield, 98.57% potency) as an off-white solid. (LC/MS) m/z (M+H)⁺=887.6. ¹H NMR (600 MHz, CD₃CN at 328 K) δ 8.29 (dd, 1H), 8.24 (s, 1H), 7.64 (dd, 1H), 7.48 (t, 1H), 7.42-7.37 (m, 2H), 7.32 (dd, 1H), 7.27-7.22 (m, 2H), 7.10-7.03 (m, 2H), 6.95 (s, 1H), 6.83 (dd, 1H), 5.08 (d, 1H), 4.54-4.42 (m, 4H), 4.33 (dd, 1H), 4.25 (s, 3H), 3.94 (dd, 2H), 3.58 (t, 2H), 3.38 (dd, 1H), 3.21-3.15 (m, 1H), 3.06 (s, 3H), 3.03-2.97 (m, 3H), 2.95 (s, 6H), 2.78 (dd, 2H), 2.74-2.56 (m, 3H), 2.07-2.01 (m, 4H), 1.46 (d, 3H), 1.42 (d, 3H), 1.24 (d, 3H).

### Example E285

### (S)—N-(4-Carbamoylbenzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide (E285)

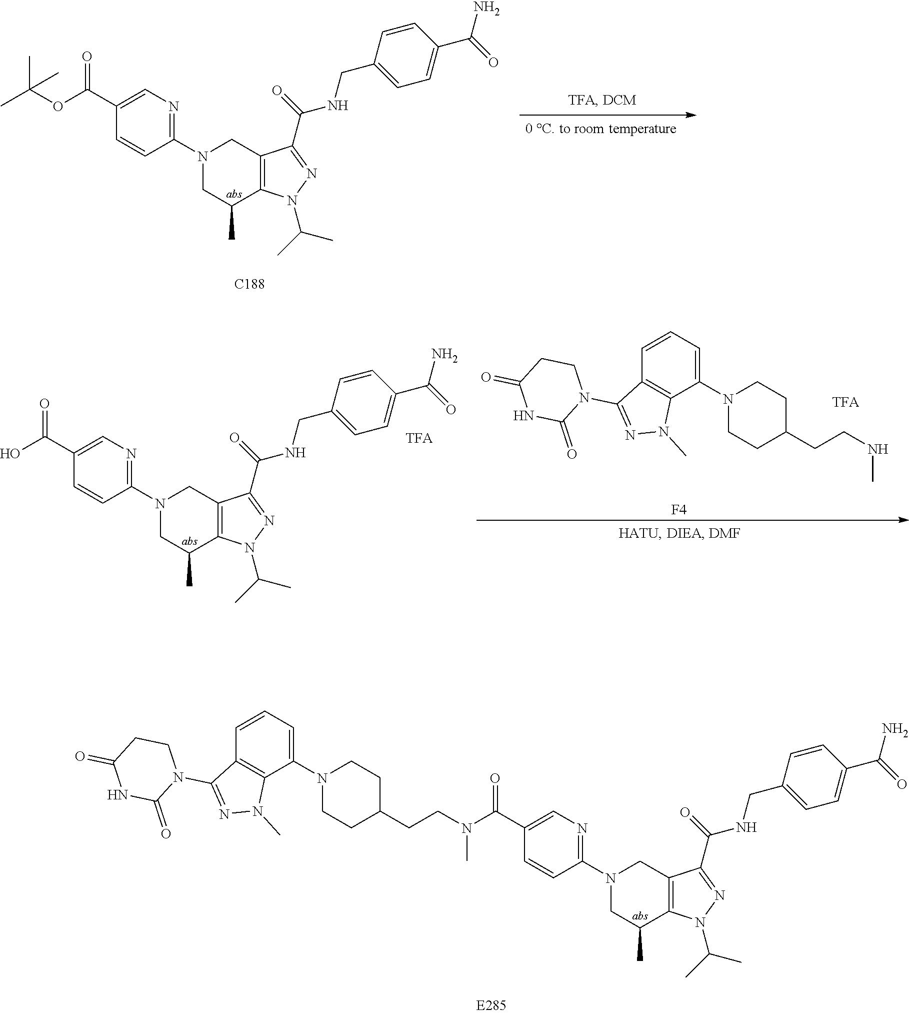

### Step 1. Preparation of (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinic acid trifluoroacetate

To a suspension of tert-butyl (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinate (1.56 g, 2.93 mmol) in DCM (12 mL) was added TFA (6 mL) at 0-5 °C. The reaction mixture was stirred at room temperature for 1 h then concentrated in vacuo. The resulting residue was lyophilized to provide (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinic acid trifluoroacetate (1.79 g, crude) as an off-white solid. The solid was used directly in the next step without further purification. (LC/MS) m/z (M+H)⁺=477.2. ¹H NMR (400 MHz, (CD₃)₂SO) δ 8.62 (d, 1H), 8.57 (t, 1H), 8.28-8.24 (m, 1H), 8.02-7.93 (m, 1H), 7.93-7.89 (m, 1H), 7.86-7.79 (m, 2H), 7.41-7.34 (m, 2H), 7.31 (s, 1H), 6.98 (d, 1H), 5.18 (d, 1H), 4.60-4.39 (m, 4H), 4.31 (d, 1H), 3.68-3.56 (m, 1H), 3.45-3.36 (m, 1H), 3.19-3.08 (m, 1H), 1.43 (dd, 6H), 1.18 (d, 3H).

### Step 2. Preparation of (S)—N-(4-carbamoylbenzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide (E285)

To a mixture of (S)-6-(3-((4-carbamoylbenzyl)carbamoyl)-1-isopropyl-7-methyl-1,4,6,7-tetrahydro-5H-pyrazolo[4,3-c]pyridin-5-yl)nicotinic acid trifluoroacetate (0.050 g, 0.085 mmol) and 1-(1-methyl-7-(4-(2-(methylamino)ethyl)piperidin-1-yl)-1H-indazol-3-yl)dihydropyrimidine-2,4(1H,3H)-dione trifluoroacetate (42 mg, 0.085 mmol) in DMF (1 mL) were added HATU (34 mg, 0.089 mmol) and DIEA (87 mg, 0.68 mmol). The resulting mixture was stirred at room temperature for 1 h then poured into H₂O (10 mL) and filtered. The filter cake was diluted with DMF (1 mL). The solution was purified by reverse phase HPLC (Boston Prime C₁₈ 200 mm×40 mm×10 μm column; Mobile Phase A: H₂O (0.05% NH₄OH+10 mM NH₄HCO₃)/Mobile Phase B: ACN, gradient: 43-63% of Mobile Phase B over 15 min; flow rate: 60 mL/min) then concentrated in vacuo and lyophilized to provide E285 (17 mg, 24% yield) as a white solid. (LC/MS) m/z (M+H)⁺=843.5. ¹H NMR (400 MHz, (CD₃)₂SO) δ 10.53 (s, 1H), 8.54 (t, 1H), 8.22 (s, 1H), 7.90 (s, 1H), 7.84-7.78 (m, 2H), 7.65-7.59 (m, 1H), 7.36 (d, 2H), 7.32-7.23 (m, 2H), 7.02-6.96 (m, 2H), 6.90 (d, 1H), 5.10 (d, 1H), 4.57-4.49 (m, 1H), 4.48-4.38 (m, 3H), 4.27-4.18 (m, 4H), 3.87 (t, 2H), 3.47-3.36 (m, 2H), 3.27-3.11 (m, 4H), 2.98 (s, 3H), 2.74 (t, 2H), 2.69-2.56 (m, 2H), 1.89-1.57 (m, 4H), 1.46-1.32 (m, 9H), 1.18 (d, 3H).

### Example E461

### rac-(R)-5-(5-((2-(4-(3-(2,6-Dioxopiperidin-3-yl)-1-methyl-1H-indazol-6-yl)piperazin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide formate (E461)

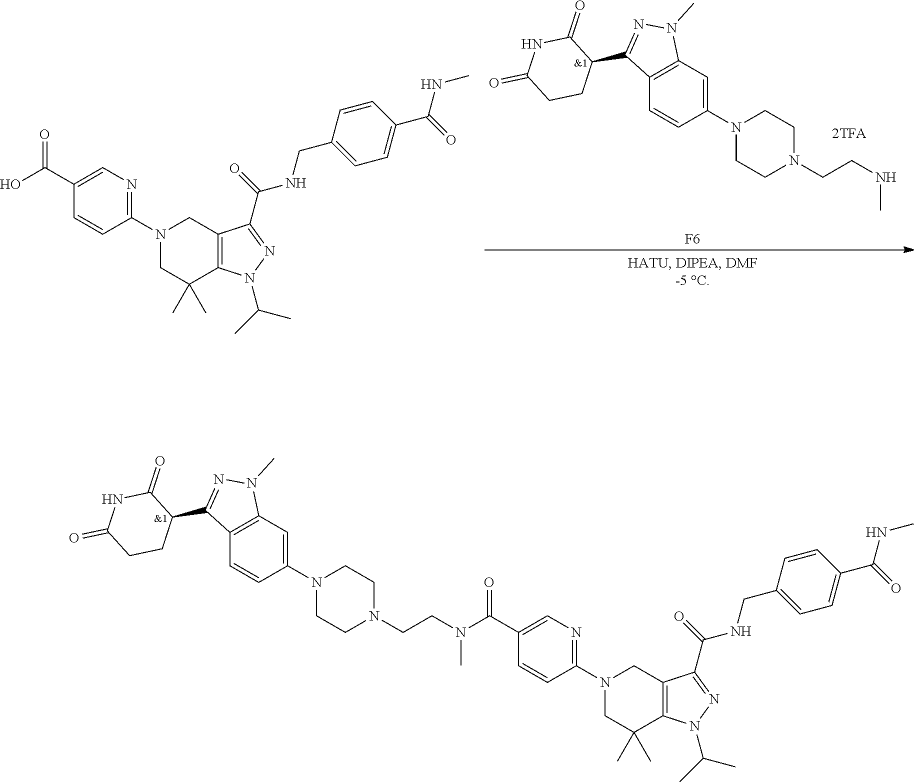

### Step 1. Preparation of rac-(R)-5-(5-((2-(4-(3-(2,6-dioxopiperidin-3-yl)-1-methyl-1H-indazol-6-yl)piperazin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide formate (E461)

At −5 °C, to a reaction mixture of rac-(R)-3-(1-methyl-6-(4-(2-(methylamino)ethyl)piperazin-1-yl)-1H-indazol-3-yl)piperidine-2,6-dione bis(2,2,2-trifluoroacetate) (0.040 g, 0.065 mmol) and 6-(1-isopropyl-7,7-dimethyl-3-((4-(methylcarbamoyl)benzyl)carbamoyl)-1,4,6,7-tetrahydro-5H-pyrazolo[4,3-c]pyridin-5-yl)nicotinic acid trifluoroacetate (0.040 g, 0.065 mmol) in DMF (1 mL) were added HATU (26 mg, 0.069 mmol) and DIEA (59 mg, 0.46 mmol). The reaction mixture was stirred at −5 °C for 1 h under N₂ gas then poured into ice-water (10 mL) which caused white solid precipitate to form. The suspension was filtered and the cake was diluted with THF (3 mL). The solution was purified by reverse phase HPLC (Welch Xtimate C₁₈ 150 mm×30 mm×5 μm; Mobile Phase: A: H₂O (0.225% formic acid); Mobile Phase B: ACN; gradient: 21-41% of Mobile Phase B over 10 min; flow rate: 30 mL/min) the lyophilized to give E461 (8.0 mg, 14% yield) as a solid. (LC/MS) m/z (M+H)⁺=871.5. ¹H NMR (400 MHz, (CD₃)₂SO) δ 10.86 (s, 1H), 8.56 (t, 1H), 8.38 (q, 1H), 8.25-8.21 (m, 1H), 7.81-7.75 (m, 2H), 7.67-7.60 (m, 1H), 7.53-7.46 (m, 1H), 7.37 (d, 2H), 6.95-6.83 (m, 3H), 4.70-4.66 (m, 3H), 4.45 (d, 2H), 4.26 (dd, 1H), 3.89 (s, 3H), 3.68 (s, 2H), 3.57-3.52 (m, 2H), 3.20-3.15 (m, 4H), 3.01 (s, 3H), 2.76 (d, 3H), 2.66-2.56 (m, 7H), 2.35-2.10 (m, 3H), 1.44 (d, 6H), 1.35 (s, 6H).

### Example E511

### (S)-5-(5-(Dimethylcarbamoyl)pyridin-2-yl)-N-(4-(4-((6-(6-(((RS)-2,6-dioxopiperidin-3-yl)carbamoyl)pyridin-2-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide (E511)

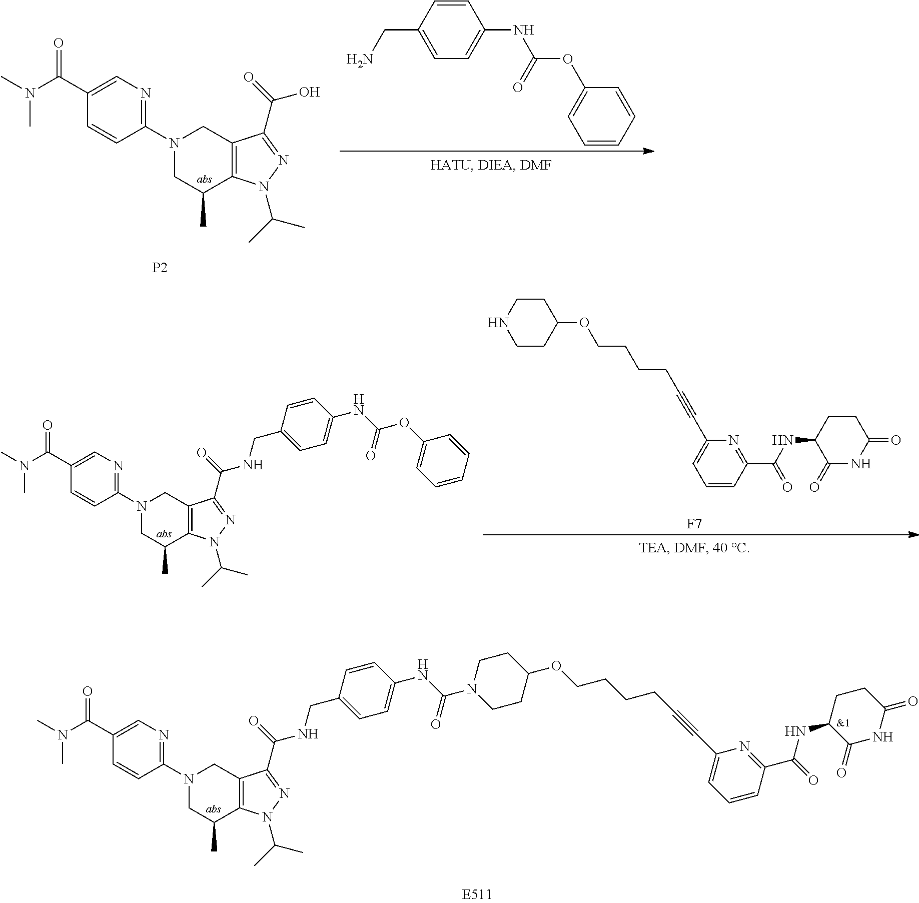

### Step 1. Preparation of phenyl (S)-(4-((5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)carbamate

To a reaction mixture of P2 (0.100 g, 0.269 mmol) in DMF (2 mL) was added HATU (0.107 g, 0.283 mmol) then stirred at room temperature for 10 min. To the suspension was added phenyl (4-(aminomethyl)phenyl)carbamate (CAS: 1019455-66-3; 97.6 mg, 0.350 mmol) and DIEA (0.139 g, 1.08 mmol). The reaction mixture was stirred at room temperature for 16 h then poured into water and filtered and concentrated in vacuo to give phenyl (S)-(4-((5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)carbamate (0.140 g, 87.3% yield) as a yellow solid. (LC/MS) m/z (M+H)⁺=596.5.

### Step 2. Preparation of (S)-5-(5-(dimethylcarbamoyl)pyridin-2-yl)-N-(4-(4-((6-(6-(((RS)-2,6-dioxopiperidin-3-yl)carbamoyl)pyridin-2-yl)hex-5-yn-1-yl)oxy)piperidine-1-carboxamido)benzyl)-1-isopropyl-7-methyl-4,5,6,7-tetrahydro-1H-pyrazolo[4,3-c]pyridine-3-carboxamide (E511)

To a solution of phenyl (S)-(4-((5-(5-(dimethylcarbamoyl)pyridin-2-yl)-1-isopropyl-7-methyl-4,5,6,7-tetrahydro-1H-pyrazolo[4,3-c]pyridine-3-carboxamido)methyl)phenyl)carbamate (0.15 g, 0.25 mmol) and F7 (0.080 g, 0.19 mmol) in DMF (2 mL) was added TEA (39 mg, 0.39 mmol) then stirred at 40 °C for 16 h. The solution was purified by reverse phase HPLC (Phenomenex Gemini NX 150 mm×30 mm×5 μm column; Mobile Phase A: water (NH₃H₂O+NH₄HCO₃)/Mobile Phase B: ACN; gradient: 31-71% of Mobile Phase B over 9 min and held at 100% of Mobile Phase B over 2 min; flow rate: 60 mL/min) to provide E511 (45 mg, 20% yield) as a white solid. (LC/MS) m/z (M+H)+=914.5. ¹H NMR (400 MHz, DMSO) δ 10.87 (s, 1H), 8.97 (d, 1H), 8.46 (s, 1H), 8.34 (t, 1H), 8.24 (d, 1H), 8.03-7.93 (m, 2H), 7.72-7.60 (m, 2H), 7.41-7.34 (m, 2H), 7.21-7.14 (m, 2H), 6.89 (d, 1H), 5.09 (d, 1H), 4.86-4.74 (m, 1H), 4.59-4.48 (m, 1H), 4.47-4.15 (m, 4H), 3.81-3.73 (m, 2H), 3.50-3.43 (m, 3H), 3.37-3.33 (m, 1H), 3.26-3.20 (m, 1H), 3.14-3.04 (m, 2H), 2.98 (s, 6H), 2.87-2.73 (m, 1H), 2.67-2.63 (m, 1H), 2.55-2.50 (m, 1H), 2.31-2.17 (m, 1H), 2.01-1.97 (m, 1H), 1.87-1.79 (m, 2H), 1.68-1.62 (m, 4H), 1.46-1.31 (m, 9H), 1.19 (d, 3H).

### Example E529

### 5-(5-((2-(4-(3-(2,4-Dioxotetrahydropyrimidin-1(2H)-yl)-1-methyl-1H-indazol-7-yl)piperazin-1-yl)ethyl)(methyl)carbamoyl)pyridin-2-yl)-1-ethyl-7,7-dimethyl-N-(4-(methylcarbamoyl)benzyl)-4,5,6,7-tetrahydro-1H-pyrazolo[4,3-c]pyridine-3-carboxamide (E529)

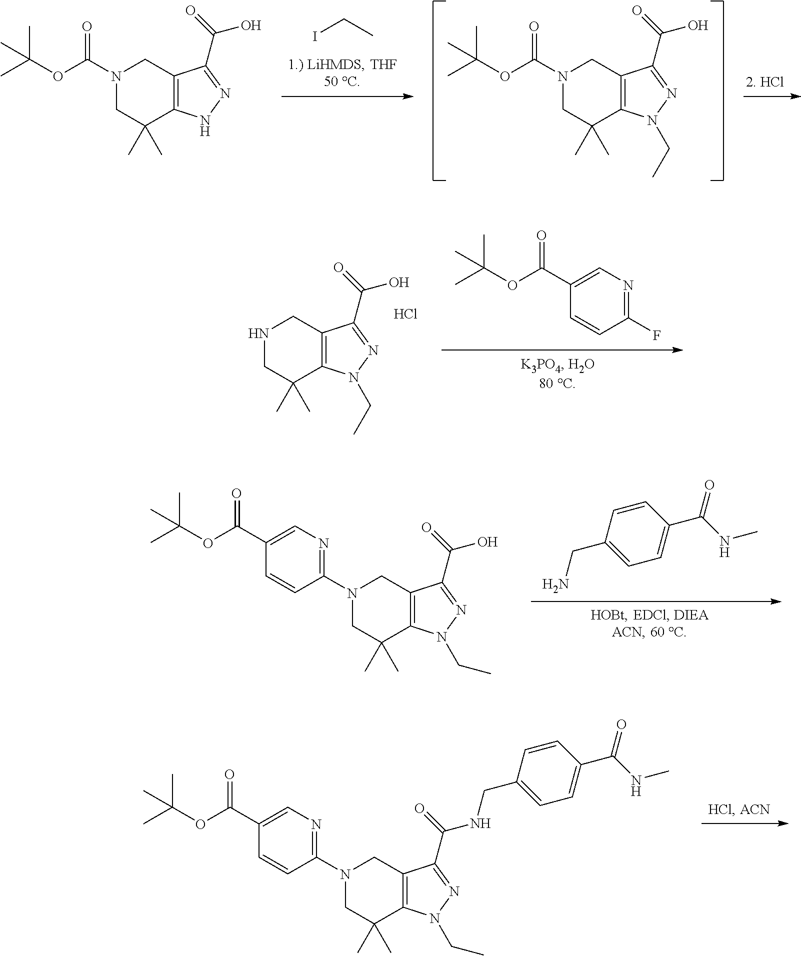

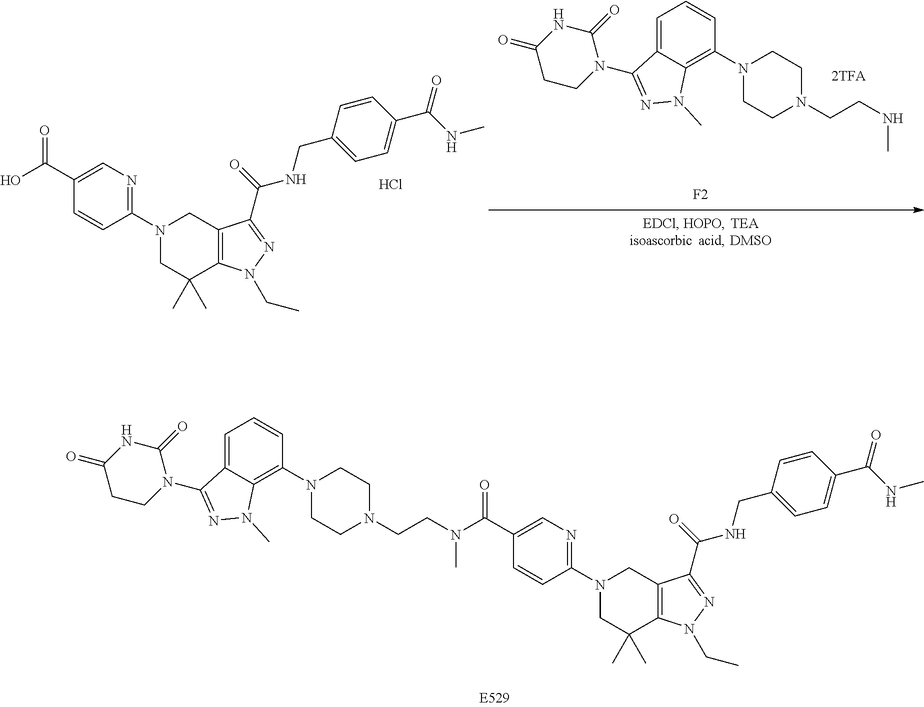

### Step 1. Preparation of 1-ethyl-7,7-dimethyl-4,5,6,7-tetrahydro-1H-pyrazolo[4,3-c]pyridine-3-carboxylic acid hydrochloride

To a solution of 5-(tert-butoxycarbonyl)-7,7-dimethyl-4,5,6,7-tetrahydro-1H-pyrazolo[4,3-c]pyridine-3-carboxylic acid (6.00 g, 20.3 mmol) in THF (60 mL) was added 1.0M LiHMDS in THF (8.84 g, 52.8 mL, 52.8 mmol). The reaction mixture was stirred for 2 h at room temperature before iodoethane (7.92 g, 4.10 mL, 50.8 mmol) was added then the reaction was stirred at 50 °C for ~1.5 h. The reaction mixture was cooled to room temperature then diluted with EtOAc and H₂O to give 5-(tert-butoxycarbonyl)-1-ethyl-7,7-dimethyl-4,5,6,7-tetrahydro-1H-pyrazolo[4,3-c]pyridine-3-carboxylic acid which was used directly in the next step. (LCMS) m/z (M+H)⁺=324.4. To the diluted solution was added 12.1 M HCl (1.93 g, 4.37 mL, 52.8 mmol). The biphasic mixture was adjusted to pH=4 with additional HCl. The mixture was extracted with EtOAc (2×). The combined organic layers were concentrated in vacuo. The resultant yellow solid was slurried in methyl ethyl ketone (40 mL) then 12.1 M HCl (2.22 g, 5.04 mL, 60.9 mmol) was added which formed a solution. The solution was concentrated in vacuo to provide 1-ethyl-7,7-dimethyl-4,5,6,7-tetrahydro-1H-pyrazolo[4,3-c]pyridine-3-carboxylic acid hydrochloride (5.28 g, crude) as a red oil. The oil was used directly in the next step without further purification. (LCMS) m/z (M+H)⁺=224.2. ¹H NMR (600 MHz, (CD₃)₂SO) δ 9.46 (s, 1H), 4.24 (q, 2H), 4.17 (t, 2H), 3.22-3.16 (m, 2H), 2.08-2.07 (m, 1H), 1.41 (s, 6H), 1.38 (t, 3H).

### Step 2. Preparation of 5-(5-(tert-butoxycarbonyl)pyridin-2-yl)-1-ethyl-7,7-dimethyl-4,5,6,7-tetrahydro-1H-pyrazolo[4,3-c]pyridine-3-carboxylic acid

To a solution of 1-ethyl-7,7-dimethyl-4,5,6,7-tetrahydro-1H-pyrazolo[4,3-c]pyridine-3-carboxylic acid hydrochloride (5.20 g, 20.0 mmol) and K₃PO₄ (12.7 g, 60.1 mmol) in H₂O (40 mL) was added tert-butyl 6-fluoronicotinate (CAS: 676560-01-3, 4.34 g, 22.0 mmol). The reaction mixture was stirred at 80 °C for ~6 h, then cooled to room temperature and diluted with MTBE. The organic layer was discarded, and the aqueous layer was neutralized with phosphoric acid (5.89 g, 3.13 mL, 60.1 mmol) in H₂O. The mixture was diluted with DCM then the organic layer was separated and filtered. The filtrate was concentrated in vacuo to provide 5-(5-(tert-butoxycarbonyl)pyridin-2-yl)-1-ethyl-7,7-dimethyl-4,5,6,7-tetrahydro-1H-pyrazolo[4,3-c]pyridine-3-carboxylic acid (4.02 g, crude) as a yellow solid. The solid was used directly in the next step without further purification. (LCMS) m/z (M+H)⁺=401.3. ¹H NMR (400 MHz, (CD₃)₂SO) δ 8.61 (d, 1H), 7.90 (dd, 1H), 6.93 (d, 1H), 4.71 (s, 2H), 4.18 (q, 2H), 3.73 (s, 2H), 1.51 (s, 9H), 1.38 (t, 3H), 1.32 (s, 6H).

### Step 3. Preparation of tert-butyl 6-(1-ethyl-7,7-dimethyl-3-((4-(methylcarbamoyl)benzyl)carbamoyl)-1,4,6,7-tetrahydro-5H-pyrazolo[4,3-c]pyridin-5-yl)nicotinate

A reaction mixture of 5-(5-(tert-butoxycarbonyl)pyridin-2-yl)-1-ethyl-7,7-dimethyl-4,5,6,7-tetrahydro-1H-pyrazolo[4,3-c]pyridine-3-carboxylic acid (0.500 g, 1.25 mmol), HOBt (0.254 g, 98% Wt, 1.62 mmol) and EDCI (0.311 g, 1.62 mmol) in ACN (6.25 mL) was stirred at room temperature for 20 min then 4-(aminomethyl)-N-methylbenzamide (CAS: 164648-64-0, 249 mg, 1.50 mmol) and DIEA (0.484 g, 0.65 mL, 3.75 mmol) were added. The reaction mixture was stirred at room temperature overnight then concentrated in vacuo. To the crude was added H₂O then extracted with DCM (3×30 mL). The combined organic layer was washed with H₂O then concentrated in vacuo. The crude product was purified by column chromatography (silica gel; 0-5% MeOH: DCM) to provide tert-butyl 6-(1-ethyl-7,7-dimethyl-3-((4-(methylcarbamoyl)benzyl)carbamoyl)-1,4,6,7-tetrahydro-5H-pyrazolo[4,3-c]pyridin-5-yl)nicotinate (435 mg, 63.7% yield) as a white foam solid. (LCMS) m/z (M+H)⁺=547.5.

### Step 4. Preparation of 6-(1-ethyl-7,7-dimethyl-3-((4-(methylcarbamoyl)benzyl)carbamoyl)-1,4,6,7-tetrahydro-5H-pyrazolo[4,3-c]pyridin-5-yl)nicotinic acid hydrochloride

To a solution of tert-butyl 6-(1-ethyl-7,7-dimethyl-3-((4-(methylcarbamoyl)benzyl)carbamoyl)-1,4,6,7-tetrahydro-5H-pyrazolo[4,3-c]pyridin-5-yl)nicotinate (0.435 g, 0.795 mmol) in ACN (4 mL) was added concentrated HCl (0.67 mL). The solution was stirred at room temperature overnight which resulted in a thick white slurry that was filtered. The filter cake was rinsed with minimal ACN (0.5 mL×5). The filter cake was collected and dried under high vacuum to give 6-(1-ethyl-7,7-dimethyl-3-((4-(methylcarbamoyl)benzyl)carbamoyl)-1,4,6,7-tetrahydro-5H-pyrazolo[4,3-c]pyridin-5-yl)nicotinic acid hydrochloride (0.352 g, crude) as a white solid. The solid was used directly in the next step without further purification. (LCMS) m/z (M+H)⁺=491.5. ¹H NMR (400 MHz, (CD₃)₂SO) 6 8.66 (t, 1H), 8.60 (d, 1H), 8.37 (q, 1H), 7.98 (dd, 1H), 7.81-7.74 (m, 2H), 7.40-7.33 (m, 2H), 7.01 (d, 1H), 4.76 (s, 3H), 4.45 (d, 2H), 4.22 (q, 2H), 3.76 (s, 2H), 2.76 (d, 3H), 1.42 (t, 3H), 1.35 (s, 6H).

### Step 5. Preparation of 5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-ethyl-7,7-dimethyl-N-(4-(methylcarbamoyl)benzyl)-4,5,6,7-tetrahydro-1H-pyrazolo[4,3-c]pyridine-3-carboxamide (E529)

A reaction mixture of 6-(1-ethyl-7,7-dimethyl-3-((4-(methylcarbamoyl)benzyl)carbamoyl)-1,4,6,7-tetrahydro-5H-pyrazolo[4,3-c]pyridin-5-yl)nicotinic acid hydrochloride (0.35 g, 0.72 mmol) and isoascorbic acid (6.4 mg, 3.9 μL, 0.036 mmol) in DMSO (2.2 mL) was stirred at room temperature for 5 min then HOPO (88 mg, 79.5 μL, 0.79 mmol) followed by EDCI (0.17 g, 0.90 mmol) were added. The mixture was stirred for 5 min at room temperature then TEA (0.37 g, 0.5 mL, 3.6 mmol) followed by 1-(1-methyl-7-(4-(2-(methylamino)ethyl)piperazin-1-yl)-1H-indazol-3-yl)dihydropyrimidine-2,4(1H,3H)-dione bis(2,2,2-trifluoroacetate) (0.48 g, 0.76 mmol) were added. The reaction mixture was stirred at room temperature for 30 min before another portion of TEA (74 mg, 0.1 mL, 0.73 mmol) was added. The reaction mixture was stirred at room temperature overnight before another portion of HOPO (17 mg, 15 mmol), EDCI (35 mg, 0.18 mmol) and TEA (22 mg, 30 μL, 0.22 mmol) were added. The reaction mixture was stirred at room temperature for 3 h and 30 min before another portion of 1-(1-methyl-7-(4-(2-(methylamino)ethyl)piperazin-1-yl)-1H-indazol-3-yl)dihydropyrimidine-2,4(1H,3H)-dione bis(2,2,2-trifluoroacetate) (23 mg, 0.037 mmol) was added. The reaction mixture was stirred at room temperature for −72 h then a pH=7 buffer solution of sodium ascorbate (0.250 g, 1.3 mmol) in pH=7 buffer (100 mL) was added dropwise then rinsed with DMSO (2×0.25 mL) and pH=7 buffer solution (1×0.25 mL). The suspension was stirred under N₂ gas for 30 min then filtered through Buchner funnel and dried under a flow of N₂ gas and high vacuum at 55 °C. The solid was purified by reverse phase HPLC (C₁₈; Mobile Phase A: ACN (0.1% formic acid)/Mobile Phase B: H₂O (0.1% formic acid); gradient: 5-50% of Mobile Phase B) to provide E529 (0.52 g, 84% yield) as a light brown solid. (LCMS) m/z (M+H)⁺=859.0. ¹H NMR (500 MHz, (CD₃)₂SO) δ 10.55 (s, 1H), 8.64 (t, 1H), 8.37 (q, 1H), 8.25 (s, 1H), 7.77 (d, 2H), 7.65 (d, 1H), 7.36 (d, 2H), 7.31 (d, 1H), 7.07-6.98 (m, 2H), 6.93 (d, 1H), 4.68 (s, 2H), 4.45 (d, 2H), 4.25-4.17 (m, 5H), 3.88 (t, 2H), 3.68 (s, 2H), 3.57-3.52 (m, 2H), 3.18-2.96 (m, 6H), 2.95-2.72 (m, 9H), 2.62-2.59 (m, 1H), 2.45-2.23 (m, 2H), 1.41 (t, 3H), 1.33 (s, 6H).

### Example E545

### (S)—N-(4-(3,3-Dimethylureido)benzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide (E545)

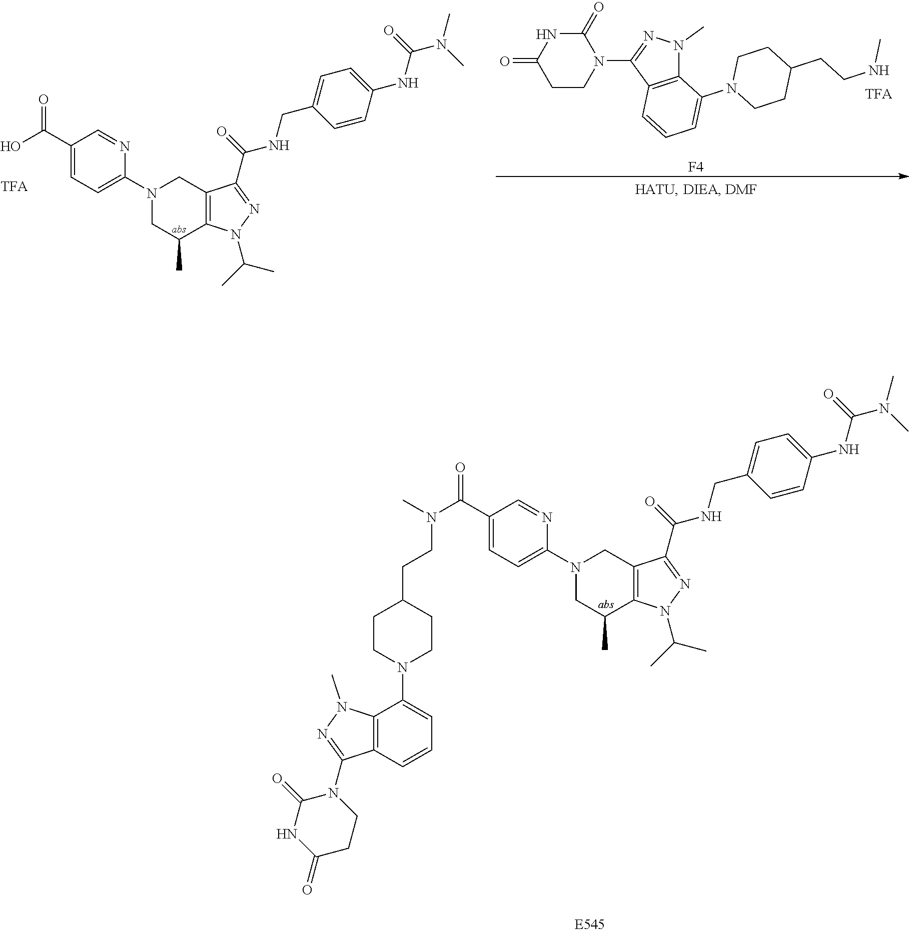

### Step 1. Preparation of (S)—N-(4-(3,3-dimethylureido)benzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide (E545)

To a stirred solution of (S)-6-(3-((4-(3,3-dimethylureido)benzyl)carbamoyl)-1-isopropyl-7-methyl-1,4,6,7-tetrahydro-5H-pyrazolo[4,3-c]pyridin-5-yl)nicotinic acid trifluoroacetate (4.89 g, 7.72 mmol), 1-(1-methyl-7-(4-(2-(methylamino)ethyl)piperidin-1-yl)-1H-indazol-3-yl)dihydropyrimidine-2,4(1H,3H)-dione trifluoroacetate (3.50 g, 7.02 mmol) and HATU (2.99 g, 7.86 mmol) in DMF (49 mL) was added DIEA (7.26 g, 56.2 mmol). The reaction mixture was stirred at room temperature for 1 h then poured into cold water (500 mL) and filtered. The filter cake was collected, solid was dissolved in DCM (50 mL) then dried over Na₂SO₄ and concentrated in vacuo. The residue was purified by reverse phase HPLC (WePure Biotech XP tC₁₈ 150 mm×40 mm×7 μm column; Mobile Phase A: H₂O (0.05% NH₄OH+10 mM NH₄HCO₃)/Mobile Phase B: ACN; gradient: 30-70% of Mobile Phase B over 11 min; flow rate: 60 mL/min). The organic phase was discarded then the aqueous phase was lyophilized to provide E545 (3.20 g, 51.4% yield) as a white solid. (LC/MS) m/z (M+H)⁺=887.5. ¹H NMR (600 MHz, CD₃CN at 328 K) δ 8.28-8.22 (m, 1H), 7.60 (dd, 1H), 7.48 (t, 1H), 7.41-7.36 (m, 2H), 7.32-7.27 (m, 1H), 7.26-7.23 (m, 2H), 7.05-6.99 (m, 2H), 6.95 (s, 1H), 6.82 (d, 1H), 5.08 (d, 1H), 4.54-4.41 (m, 4H), 4.37-4.30 (m, 1H), 4.25 (s, 3H), 3.93 (t, 2H), 3.50 (t, 2H), 3.38 (dd, 1H), 3.26-3.14 (m, 3H), 3.02 (s, 3H), 2.95 (s, 6H), 2.78 (t, 2H), 2.75-2.65 (m, 2H), 1.82-1.71 (m, 2H), 1.65 (q, 2H), 1.53-1.40 (m, 1OH), 1.24 (d, 3H).

### Example E618

### (S)-5-(3-(4-(4-(2-(3-((RS)-2,6-Dioxopiperidin-3-yl)-1-methyl-1H-indazol-7-yl)ethyl)piperazin-1-yl)phenyl)-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (E618)

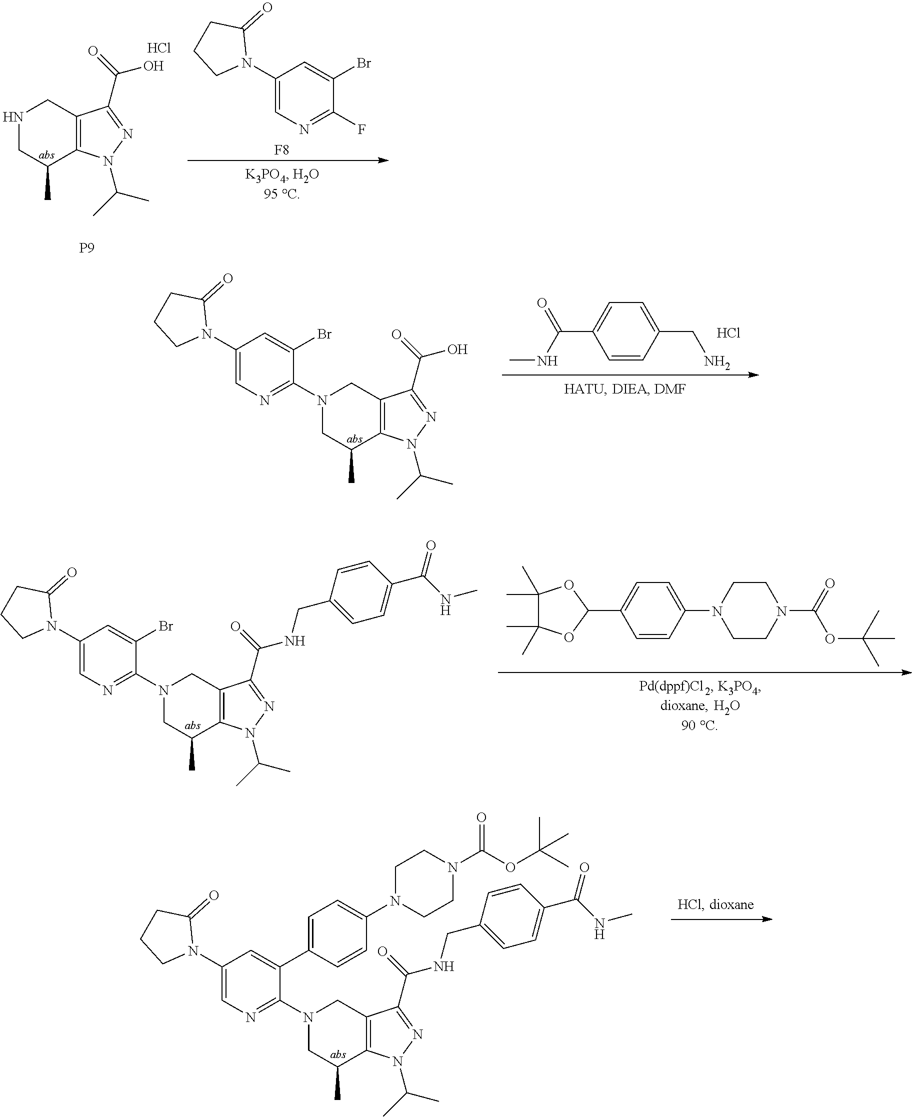

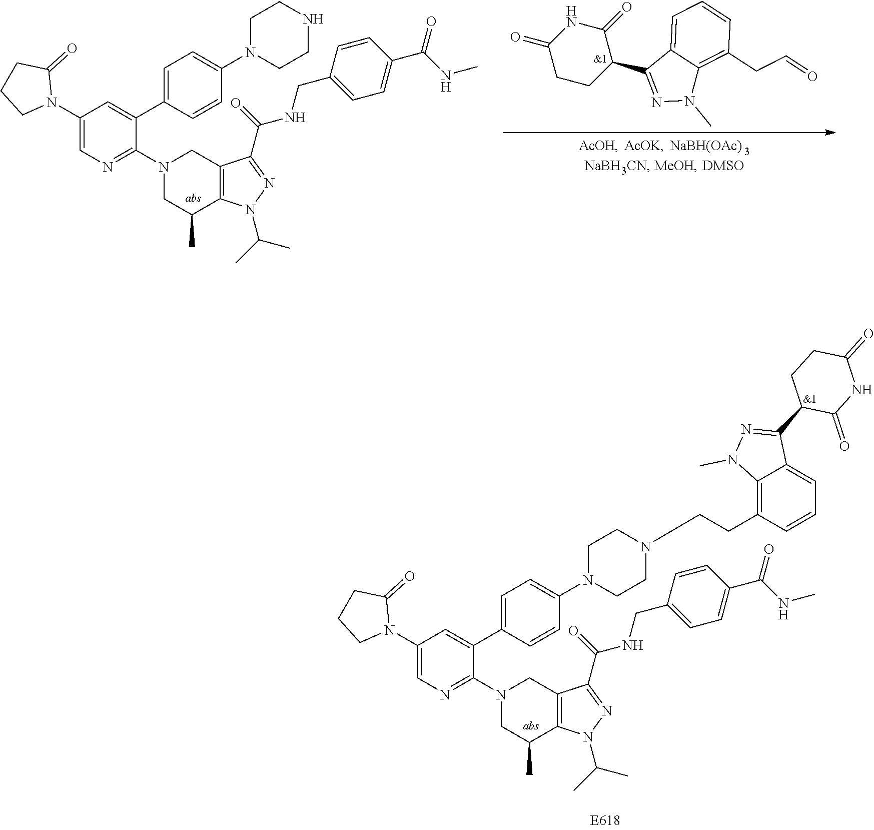

### Step 1. Preparation of (S)-5-(3-bromo-5-(2-oxopyrrolidin-1-yl)pyridin-2-yl)-1-isopropyl-7-methyl-4,5,6,7-tetrahydro-1H-pyrazolo[4,3-c]pyridine-3-carboxylic acid

To a stirred solution of F8 (2.45 g, 9.46 mmol) and P9 (2.46 g, 9.46 mmol) in H₂O (25 mL) was added K₃PO₄ (6.02 g, 28.4 mmol). The reaction mixture was stirred at 95 °C for 16 h then diluted with H₂O (80 mL) and extracted with EtOAc (3×60 mL). The organic layers were discarded. The aqueous phase was adjusted to pH ~5 with 1 M HCl then extracted with EtOAc (3×50 mL). The combined organic layers were dried over Na₂SO₄, filtered, and concentrated in vacuo to afford (S)-5-(3-bromo-5-(2-oxopyrrolidin-1-yl)pyridin-2-yl)-1-isopropyl-7-methyl-4,5,6,7-tetrahydro-1H-pyrazolo[4,3-c]pyridine-3-carboxylic acid (2.35 g, 53.7% yield) as a yellow solid. (LC/MS) m/z (M+H)⁺=464.0.

### Step 2. Preparation of (S)-5-(3-bromo-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide

To a solution of (S)-5-(3-bromo-5-(2-oxopyrrolidin-1-yl)pyridin-2-yl)-1-isopropyl-7-methyl-4,5,6,7-tetrahydro-1H-pyrazolo[4,3-c]pyridine-3-carboxylic acid (1.90 g, 4.11 mmol) and 4-(aminomethyl)-N-methylbenzamide hydrochloride (CAS: 1158467-80-1; 825 mg, 4.11 mmol) in DMF (20 mL) were added HATU (1.59 g, 4.19 mmol) and DIEA (2.12 g, 16.4 mmol). The reaction mixture was stirred at room temperature for 2 h then poured into H₂O (80 mL) and extracted with EtOAc (3×45 mL). The combined organic layers were washed with brine (2×50 mL), dried over Na₂SO₄, filtered, and concentrated in vacuo. The residue was purified by column chromatography (silica gel; 0-10% MeOH: DCM) to provide (S)-5-(3-bromo-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (0.820 g, 32.8% yield) as a white solid. (LC/MS) m/z (M+H)⁺=610.2. ¹H NMR (400 MHz, (CD₃)₂SO) 6 8.53-8.42 (m, 3H), 8.36 (q, 1H), 7.81-7.74 (m, 2H), 7.40-7.33 (m, 2H), 4.61-4.50 (m, 2H), 4.45 (d, 2H), 4.16 (d, 1H), 3.87-3.79 (m, 2H), 3.50 (dd, 1H), 3.39-3.34 (m, 1H), 3.29-3.21 (m, 1H), 2.77 (d, 3H), 2.51-2.44 (m, 2H), 2.13-2.01 (m, 2H), 1.47 (d, 3H), 1.42 (d, 3H), 1.33 (d, 3H).

### Step 3. Preparation of tert-butyl (S)-4-(4-(2-(1-isopropyl-7-methyl-3-((4-(methylcarbamoyl)benzyl)carbamoyl)-1,4,6,7-tetrahydro-5H-pyrazolo[4,3-c]pyridin-5-yl)-5-(2-oxopyrrolidin-1-yl)pyridin-3-yl)phenyl)piperazine-1-carboxylate

To a mixture of (S)-5-(3-bromo-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (0.500 g, 0.822 mmol), tert-butyl 4-(4-(4,4,5,5-tetramethyl-1,3-dioxolan-2-yl)phenyl)piperazine-1-carboxylate (CAS: 470478-90-1; 319 mg, 0.822 mmol), and K₃PO₄ (947 mg, 2.46 mmol) in dioxane (8 mL) and H₂O (2 mL) was added Pd(dppf)Cl₂ (60.1 mg, 0.0822 mmol) under a nitrogen gas. The reaction mixture was stirred at 100 °C for 3 h then H₂O (20 mL) was added and extracted with EtOAc (3×30 mL). The combined organic layers were washed with brine (2×30 mL), dried over Na₂SO₄, filtered, and concentrated in vacuo. The brown gum residue was purified by column chromatography (silica gel; 0-10% MeOH: DCM) to provide tert-butyl (S)-4-(4-(2-(1-isopropyl-7-methyl-3-((4-(methylcarbamoyl)benzyl)carbamoyl)-1,4,6,7-tetrahydro-5H-pyrazolo[4,3-c]pyridin-5-yl)-5-(2-oxopyrrolidin-1-yl)pyridin-3-yl)phenyl)piperazine-1-carboxylate (0.610 g, 94.0% yield) as a yellow solid. (LC/MS) m/z (M+H)⁺=790.5.

### Step 4. Preparation of (S)-1-isopropyl-7-methyl-N-(4-(methylcarbamoyl)benzyl)-5-(5-(2-oxopyrrolidin-1-yl)-3-(4-(piperazin-1-yl)phenyl)pyridin-2-yl)-4,5,6,7-tetrahydro-1H-pyrazolo[4,3-c]pyridine-3-carboxamide

To a solution of tert-butyl (S)-4-(4-(2-(1-isopropyl-7-methyl-3-((4-(methylcarbamoyl)benzyl)carbamoyl)-1,4,6,7-tetrahydro-5H-pyrazolo[4,3-c]pyridin-5-yl)-5-(2-oxopyrrolidin-1-yl)pyridin-3-yl)phenyl)piperazine-1-carboxylate (0.610 g, 0.772 mmol) in DCM (5 mL) was added 2M HCl in dioxane (10 mL) at −78 °C. The reaction mixture was warmed to room temperature and stirred for 2 h. The reaction mixture was then concentrated in vacuo to afford (S)-1-isopropyl-7-methyl-N-(4-(methylcarbamoyl)benzyl)-5-(5-(2-oxopyrrolidin-1-yl)-3-(4-(piperazin-1-yl)phenyl)pyridin-2-yl)-4,5,6,7-tetrahydro-1H-pyrazolo[4,3-c]pyridine-3-carboxamide (0.580 g, crude) as a yellow solid. The solid was used directly into the next step without further purification. (LC/MS) m/z (M+H)⁺=690.4.

### Step 5. Preparation of (S)-5-(3-(4-(4-(2-(3-((RS)-2,6-dioxopiperidin-3-yl)-1-methyl-1H-indazol-7-yl)ethyl)piperazin-1-yl)phenyl)-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (E618)

To a solution of (S)-1-isopropyl-7-methyl-N-(4-(methylcarbamoyl)benzyl)-5-(5-(2-oxopyrrolidin-1-yl)-3-(4-(piperazin-1-yl)phenyl)pyridin-2-yl)-4,5,6,7-tetrahydro-1H-pyrazolo[4,3-c]pyridine-3-carboxamide (0.060 g, 0.083 mmol) and rac-(R)-2-(3-(2,6-dioxopiperidin-3-yl)-1-methyl-1H-indazol-7-yl)acetaldehyde (CAS: 3077378-47-0; 47 mg, 0.16 mmol) in DMSO (1 mL) and MeOH (0.1 mL) were added AcOK (28 mg, 0.29 mmol) and AcOH (9.9 mg, 0.16 mmol), followed by NaBH(OAc)₃ (35 mg, 0.16 mmol). The reaction mixture was degassed with nitrogen gas for 2 min and stirred at room temperature for 16 h. An additional portion of rac-(R)-2-(3-(2,6-dioxopiperidin-3-yl)-1-methyl-1H-indazol-7-yl)acetaldehyde (CAS: 3077378-47-0; 24 mg, 0.083 mmol) and NaBH₃CN (0.010 g, 0.16 mmol) were added under nitrogen gas. The reaction mixture was stirred at room temperature for an additional 16 h then quenched with H₂O (1 mL). The residue was purified by reverse phase HPLC (WePure Biotech XP tC₁₈ 150 mm×40 mm×7 μm column, Mobile Phase A: H₂O (10 mM NH₄HCO₃)/Mobile Phase B: ACN, gradient: 29-59% of Mobile Phase B over 10 min; flow rate: 60 mL/min) to give (S)-5-(3-(4-(4-(2-(3-((RS)-2,6-dioxopiperidin-3-yl)-1-methyl-1H-indazol-7-yl)ethyl)piperazin-1-yl)phenyl)-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (17 mg, 22% yield) as a solid. (LC/MS) m/z (M+H)⁺=960.5. ¹H NMR (400 MHz, (CD₃)₂SO) δ 10.90 (s, 1H), 8.45-8.34 (m, 3H), 7.89 (d, 1H), 7.81-7.75 (m, 2H), 7.61-7.54 (m, 1H), 7.43-7.32 (m, 4H), 7.22 (d, 1H), 7.09-6.99 (m, 3H), 4.52-4.33 (m, 5H), 4.25 (s, 3H), 4.08 (d, 1H), 3.90-3.80 (m, 2H), 3.31-3.10 (m, 8H), 3.00-2.96 (m, 1H), 2.80-2.72 (m, 3H), 2.72-2.58 (m, 8H), 2.50-2.44 (m, 1H), 2.41-2.30 (m, 1H), 2.22-2.02 (m, 3H), 1.42 (d, 3H), 1.37 (d, 3H), 1.25 (s, 1H), 0.94 (d, 3H).

### Example E619

### (S)-5-(3-(4-(4-(2-(1-((RS)-2,6-Dioxopiperidin-3-yl)-3-methyl-2-oxo-2,3-dihydro-1H-benzo[d]imidazol-5-yl)ethyl)piperazin-1-yl)phenyl)-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (E619)

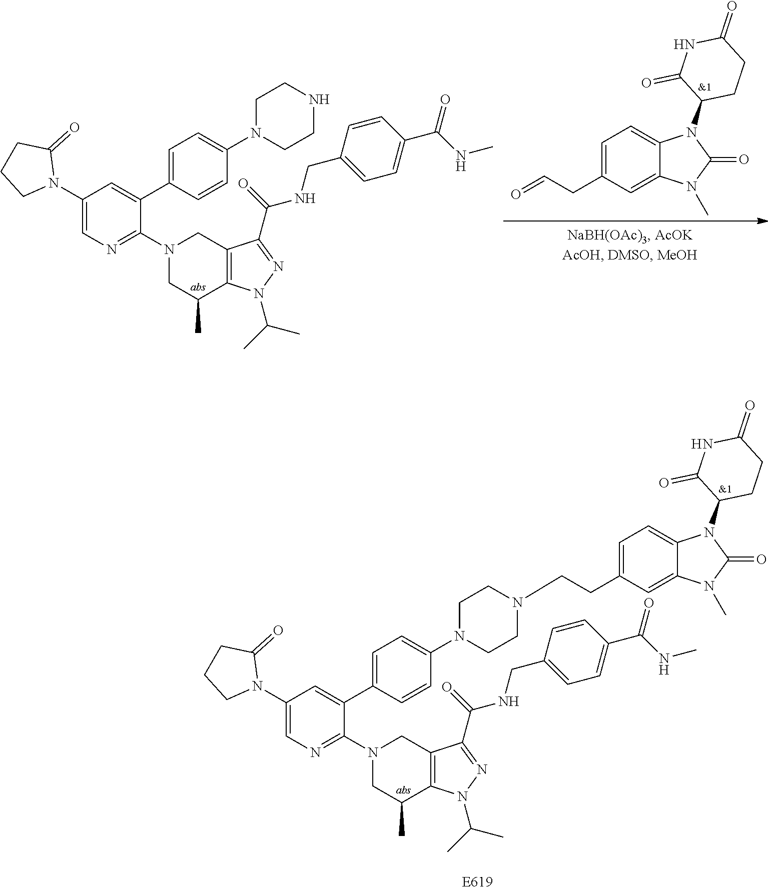 [SMILES: CNC(=O)c1ccc(CNC(=O)c2nn(C(C)C)c3c2CN(c2ncc(N4CCCC4=O)cc2-c2ccc(N4CCNCC4)cc2)C[C@@H]3C)cc1 ; Cn1c(=O)n([C@@H]2CCC(=O)NC2=O)c2ccc(CC=O)cc21 ; CNC(=O)c1ccc(CNC(=O)c2nn(C(C)C)c3c2CN(c2ncc(N4CCCC4=O)cc2-c2ccc(N4CCN(CCc5ccc6c(c5)n(C)c(=O)n6[C@@H]5CCC(=O)NC5=O)CC4)cc2)C[C@@H]3C)cc1]

### Step 5. Preparation of (S)-5-(3-(4-(4-(2-(1-((RS)-2,6-dioxopiperidin-3-yl)-3-methyl-2-oxo-2,3-dihydro-1H-benzo[d]imidazol-5-yl)ethyl)piperazin-1-yl)phenyl)-5-(2-oxopyrrolidin-1-yl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide (E619)

To a solution of (S)-1-isopropyl-7-methyl-N-(4-(methylcarbamoyl)benzyl)-5-(5-(2-oxopyrrolidin-1-yl)-3-(4-(piperazin-1-yl)phenyl)pyridin-2-yl)-4,5,6,7-tetrahydro-1H-pyrazolo[4,3-c]pyridine-3-carboxamide (0.060 g, 0.083 mmol) and rac-(R)-2-(1-(2,6-dioxopiperidin-3-yl)-3-methyl-2-oxo-2,3-dihydro-1H-benzo[d]imidazol-5-yl)acetaldehyde (CAS: 2408505-12-2; 87 mg, 0.16 mmol) in DMSO (1 mL) and MeOH (0.1 mL) were added AcOK (28 mg, 0.29 mmol) and AcOH (9.9 mg, 0.16 mmol), followed by NaBH(OAc)₃ (35 mg, 0.16 mmol). The reaction mixture was degassed with nitrogen gas for 2 min and stirred at room temperature for 16 h then poured into H₂O (0.1 mL). The suspension was filtered. The filtrate was purified by reverse phase HPLC (WePure Biotech XP tC₁₈ 150 mm×40 mm×7 μm column; Mobile Phase A: H₂O (0.05% NH₄OH+10 mM NH₄HCO₃)/Mobile Phase B: ACN; gradient: 26-66% of Mobile Phase B over 11 min; flow rate: 60 mL/min) and lyophilized to provide E619 (21 mg, 26% yield) as a white solid. (LC/MS) m/z (M+H)⁺=975.6. ¹H NMR (400 MHz, (CD₃)₂SO) δ 11.11 (s, 1H), 8.48-8.30 (m, 3H), 7.88 (d, 1H), 7.76 (d, 2H), 7.41-7.30 (m, 4H), 7.10 (s, 1H), 7.05-6.90 (m, 4H), 5.34 (dd, 1H), 4.50-4.37 (m, 4H), 4.05 (d, 1H), 3.88-3.78 (m, 2H), 3.36-3.30 (m, 4H), 3.21-3.08 (m, 6H), 2.99-2.53 (m, 14H), 2.48-2.39 (m, 2H), 2.13-1.96 (m, 3H), 1.40 (d, 3H), 1.36 (d, 3H), 0.92 (d, 3H).

### Example E710

### rac-(R)—N-(4-(3,3-Dimethylureido)benzyl)-5-(5-((2-(4-(4-(2,6-dioxopiperidin-3-yl)-2-fluorophenyl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide (E710)

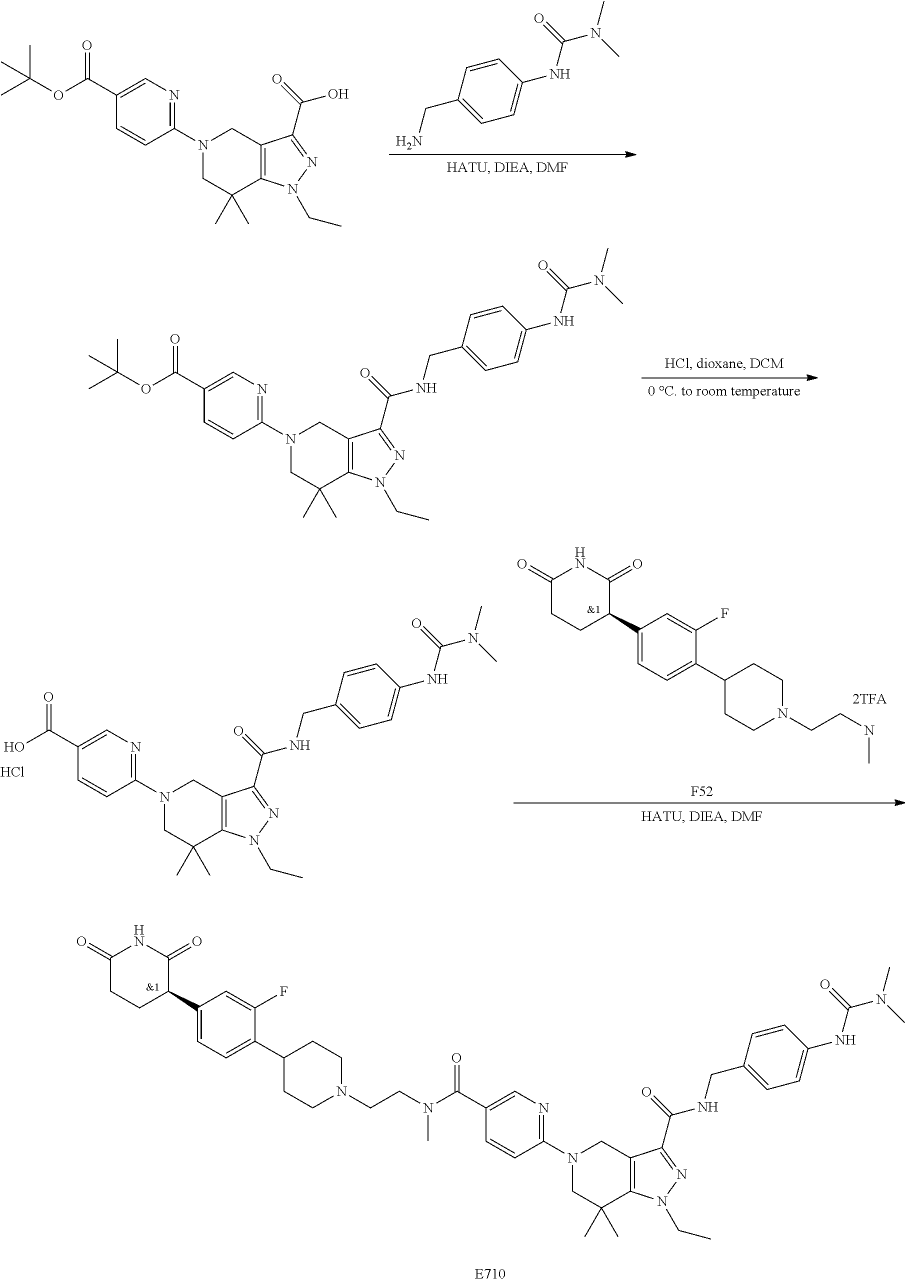

### Step 1. Preparation of tert-butyl 6-(3-((4-(3,3-dimethylureido)benzyl)carbamoyl)-1-ethyl-7,7-dimethyl-1,4,6,7-tetrahydro-5H-pyrazolo[4,3-c]pyridin-5-yl)nicotinate

To a solution of 5-(5-(tert-butoxycarbonyl)pyridin-2-yl)-1-ethyl-7,7-dimethyl-4,5,6,7-tetrahydro-1H-pyrazolo[4,3-c]pyridine-3-carboxylic acid (2.00 g, 4.99 mmol) and 3-(4-(aminomethyl)phenyl)-1,1-dimethylurea hydrochloride (1.15 g, 4.99 mmol) in DMF (20 mL) were added HATU (1.99 g, 5.24 mmol) and DIEA (2.58 g, 20.0 mmol). The reaction mixture was stirred at room temperature under nitrogen for 3 h. The resulting mixture was slowly added to ice-water (80 mL), which resulted in the formation of precipitate. The suspension was filtered then the filter cake was collected and lyophilized to give tert-butyl 6-(3-((4-(3,3-dimethylureido)benzyl)carbamoyl)-1-ethyl-7,7-dimethyl-1,4,6,7-tetrahydro-5H-pyrazolo[4,3-c]pyridin-5-yl)nicotinate (2.85 g, crude) as a white solid. The solid was used directly in the next step without further purification. (LC/MS) m/z (M+H)⁺=576.6.

### Step 2. Preparation of 6-(3-((4-(3,3-dimethylureido)benzyl)carbamoyl)-1-ethyl-7,7-dimethyl-1,4,6,7-tetrahydro-5H-pyrazolo[4,3-c]pyridin-5-yl)nicotinic acid hydrochloride

At 0 °C, to a solution of tert-butyl 6-(3-((4-(3,3-dimethylureido)benzyl)carbamoyl)-1-ethyl-7,7-dimethyl-1,4,6,7-tetrahydro-5H-pyrazolo[4,3-c]pyridin-5-yl)nicotinate (2.85 g, 4.95 mmol) in DCM (20 mL) was added 1 M HCl in dioxane (40 mL, 4.95 mmol). The reaction mixture was stirred at room temperature for 6 h then concentrated under reduced pressure. The resulting residue was lyophilized to afford 6-(3-((4-(3,3-dimethylureido)benzyl)carbamoyl)-1-ethyl-7,7-dimethyl-1,4,6,7-tetrahydro-5H-pyrazolo[4,3-c]pyridin-5-yl)nicotinic acid hydrochloride (2.80 g, crude) as a white solid. The solid was used directly in the next step without further purification. (LC/MS) m/z (M+H)⁺=520.3. ¹H NMR (400 MHz, (CD₃)₂SO) δ 8.62 (d, 1H), 8.47-8.39 (m, 1H), 8.21 (s, 1H), 7.93 (dd, 1H), 7.36 (d, 2H), 7.14 (d, 2H), 6.93 (d, 1H), 4.73 (s, 2H), 4.31 (d, 2H), 4.19 (q, 2H), 3.72 (s, 2H), 2.89 (s, 6H), 1.39 (t, 3H), 1.32 (s, 6H).

### Step 3. Preparation of rac-(R)—N-(4-(3,3-dimethylureido)benzyl)-5-(5-((2-(4-(4-(2,6-dioxopiperidin-3-yl)-2-fluorophenyl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide (E710)

To a stirred solution of F52 (0.050 g, 0.087 mmol), 6-(3-((4-(3,3-dimethylureido)benzyl)carbamoyl)-1-ethyl-7,7-dimethyl-1,4,6,7-tetrahydro-5H-pyrazolo[4,3-c]pyridin-5-yl)nicotinic acid hydrochloride (48 mg, 0.087 mmol) and HATU (35 mg, 0.091 mmol) in DMF (1 mL) was added DIEA (0.10 g, 0.78 mmol). The reaction mixture was stirred at room temperature for 2 h then poured into H₂O. The suspension was filtered and the filter cake was collected. The filter cake was purified by reverse phase HPLC (WePure Biotech XP tC₁₈ column (150 mm×40 mm×7 μm), Mobile Phase A: H₂O (0.225% formic acid) Mobile Phase B: ACN; gradient: 14-54% of Mobile Phase B over 11 min; flow rate: 60 mL/min) to give rac-(R)—N-(4-(3,3-dimethylureido)benzyl)-5-(5-((2-(4-(4-(2,6-dioxopiperidin-3-yl)-2-fluorophenyl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide (16 mg, 21% yield) as a white solid. (LC/MS) m/z (M+H)⁺=850.2. ¹H NMR (400 MHz, (CD₃)₂SO) δ 10.85 (s, 1H), 8.44 (t, 1H), 8.25-8.20 (m, 2H), 7.63 (dd, 1H), 7.41-7.34 (m, 2H), 7.27 (t, 1H), 7.19-7.13 (m, 2H), 7.06-6.98 (m, 2H), 6.92 (d, 1H), 4.68 (s, 2H), 4.33 (d, 2H), 4.20 (q, 2H), 3.85 (dd, 1H), 3.68 (s, 2H), 3.51-3.47 (m, 2H), 2.99 (s, 3H), 2.90 (s, 6H), 2.75-2.59 (m, 2H), 2.48-2.43 (m, 2H), 2.27-2.15 (m, 1H), 2.05-1.99 (m, 4H), 1.68-1.63 (m, 5H), 1.40 (t, 3H), 1.33 (s, 6H).

### Example E726

(S)-1-(2-((3-(3-(2,4-Dioxotetrahydropyrimidin-1(2H)-yl)-1-methyl-1H-indazol-6-yl)prop-2-yn-1-yl)oxy)ethyl)-7-methyl-5-(5-(1-methyl-1H-imidazol-2-yl)pyridin-2-yl)-N-(4-(methylcarbamoyl)benzyl)-4,5,6,7-tetrahydro-1H-pyrazolo[4,3-c]pyridine-3-carboxamide (E726)

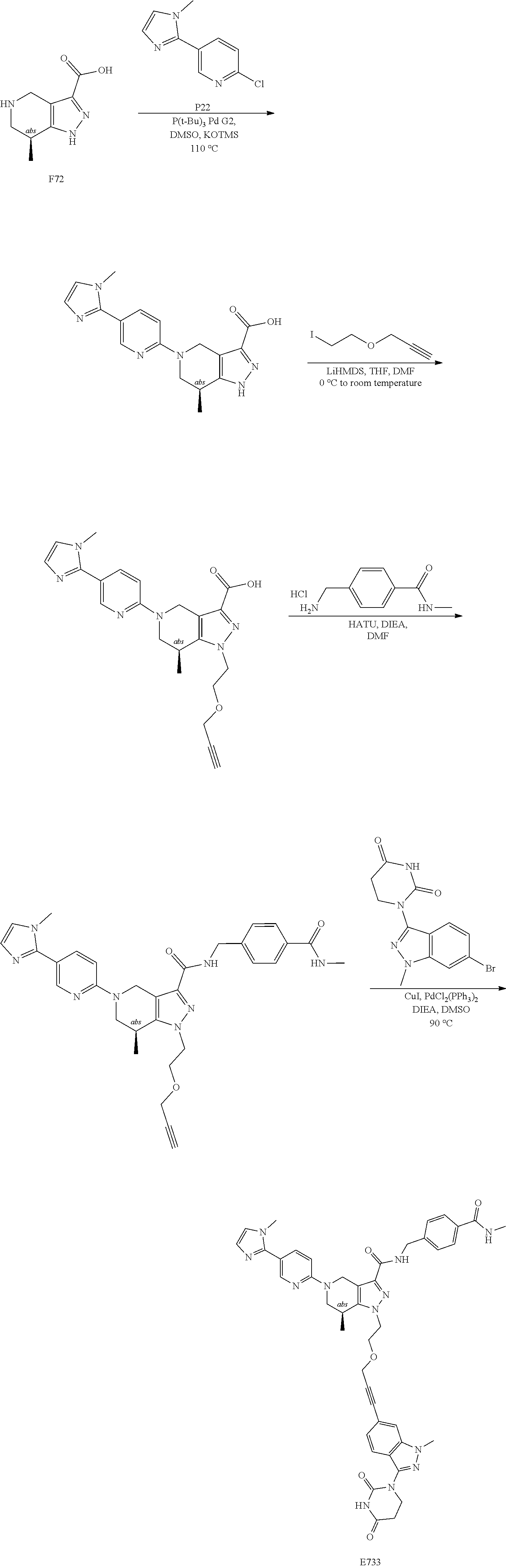

### Step 1. Preparation of (S)-7-methyl-5-(5-(1-methyl-1H-imidazol-2-yl)pyridin-2-yl)-4,5,6,7-tetrahydro-1H-pyrazolo[4,3-c]pyridine-3-carboxylic acid

To a solution of (S)-7-methyl-4,5,6,7-tetrahydro-1H-pyrazolo[4,3-c]pyridine-3-carboxylic acid hydrochloride (0.500 g, 2.30 mmol) and P22 (534 mg, 2.76 mmol) in DMSO (10 mL) were added KOTMS (1.03 g, 8.04 mmol) and P(t-Bu)₃ Pd G2 (235 mg, 0.459 mmol) at room temperature under a nitrogen atmosphere. The reaction mixture was stirred at 110 °C for 16 h then poured into H₂O (10 mL) and filtered. The filter cake was discarded. The aqueous phase was acidified with 1 M HCl then was extracted with (1:10, MeOH: DCM) (3×15 mL). The organic phases were discarded, and the remaining aqueous phase was subjected to reverse phase HPLC (WePure Biotech XP tC₁₈ 150 mm×40 mm×7 μm column; Mobile Phase A: H₂O (0.225% formic acid)/Mobile Phase B: ACN; gradient: 0-29% of Mobile Phase B over 11 min; flow rate: 60 mL/min) and lyophilized to afford (S)-7-methyl-5-(5-(1-methyl-1H-imidazol-2-yl)pyridin-2-yl)-4,5,6,7-tetrahydro-1H-pyrazolo[4,3-c]pyridine-3-carboxylic acid (331 mg, 42.6% yield) as a yellow solid. (LC/MS) m/z (M+H)⁺=339.1.

### Step 2. Preparation of (S)-7-methyl-5-(5-(1-methyl-1H-imidazol-2-yl)pyridin-2-yl)-1-(2-(prop-2-yn-1-yloxy)ethyl)-4,5,6,7-tetrahydro-1H-pyrazolo[4,3-c]pyridine-3-carboxylic acid

At 0-10 °C, to a solution of (S)-7-methyl-5-(5-(1-methyl-1H-imidazol-2-yl)pyridin-2-yl)-4,5,6,7-tetrahydro-1H-pyrazolo[4,3-c]pyridine-3-carboxylic acid (0.150 g, 0.443 mmol) in DMF (2.70 mL) was added 1 M LiHMDS in THF (223 mg, 1.33 mL, 1.33 mmol) dropwise under nitrogen gas. The reaction mixture was stirred at room temperature for 5 h then cooled to 0-10 °C before 3-(2-iodoethoxy)prop-1-yne (CAS: 164118-56-3; 0.140 g, 0.665 mmol) was added. The reaction mixture was stirred at room temperature under nitrogen gas for 18 h then cooled to 0 °C before a solution of 3-(2-iodoethoxy)prop-1-yne (14.0 mg, 0.0665 mmol) in DMF (0.2 mL) was added. The reaction mixture was stirred at room temperature for 22 h then cooled to 0 °C and quenched with H₂O (30 mL). The aqueous phase was extracted with EtOAc (2×20 mL) then the organic layers were discarded. The aqueous layer was acidified to pH=5-6 with 1 M aqueous HCl then extracted with (1:10, MeOH: DCM)(8×30 mL). The combined organic layers were dried over Na₂SO₄ and concentrated under reduced pressure to afford (S)-7-methyl-5-(5-(1-methyl-1H-imidazol-2-yl)pyridin-2-yl)-1-(2-(prop-2-yn-1-yloxy)ethyl)-4,5,6,7-tetrahydro-1H-pyrazolo[4,3-c]pyridine-3-carboxylic acid (0.120 g, 64.4%) as a yellow gum. (LC/MS) m/z (M+H)⁺=421.2. ¹H NMR (400 MHz, (CD₃)₂SO) 6 12.79 (br s, 1H), 8.44 (d, 1H), 7.85 (dd, 1H), 7.23-7.18 (m, 1H), 6.99 (d, 1H), 6.93 (d, 1H), 5.07 (d, 1H), 4.42-4.19 (m, 4H), 4.12 (d, 2H), 3.91-3.80 (m, 2H), 3.74-3.69 (m, 3H), 3.45 (t, 1H), 3.27-3.19 (m, 2H), 1.23 (d, 3H).

### Step 3. Preparation of (S)-7-methyl-5-(5-(1-methyl-1H-imidazol-2-yl)pyridin-2-yl)-N-(4-(methylcarbamoyl)benzyl)-1-(2-(prop-2-yn-1-yloxy)ethyl)-4,5,6,7-tetrahydro-1H-pyrazolo[4,3-c]pyridine-3-carboxamide

The same reaction was conducted in two batches then combined for purification. To a mixture of (S)-7-methyl-5-(5-(1-methyl-1H-imidazol-2-yl)pyridin-2-yl)-1-(2-(prop-2-yn-1-yloxy)ethyl)-4,5,6,7-tetrahydro-1H-pyrazolo[4,3-c]pyridine-3-carboxylic acid (0.10 g, 0.18 mmol) in DMF (2 mL) was added HATU (71 mg, 0.19 mmol) at room temperature under nitrogen gas to form the first batch. The reaction mixture was stirred at room temperature for 15 min before 4-(aminomethyl)-N-methylbenzamide hydrochloride (CAS: 1158467-80-1; 35 mg, 0.17 mmol) and DIEA (69 mg, 0.53 mmol) were then added to the first batch. The reaction mixture of the first batch was stirred at room temperature for 4 h.

A second batch of the same reaction was conducted with (S)-7-methyl-5-(5-(1-methyl-1H-imidazol-2-yl)pyridin-2-yl)-1-(2-(prop-2-yn-1-yloxy)ethyl)-4,5,6,7-tetrahydro-1H-pyrazolo[4,3-c]pyridine-3-carboxylic acid (0.020 g, 0.38 mmol). The two batches were combined then quenched with H₂O (30 mL) and extracted with EtOAc (3×30 mL). The combined organic layers were washed with brine (2×30 mL), dried over Na₂SO₄, filtered, and concentrated under reduced pressure to afford a yellow gum. The residue was purified by column chromatography (silica gel; gradient: 0-20% MeOH: DCM) to provide (S)-7-methyl-5-(5-(1-methyl-1H-imidazol-2-yl)pyridin-2-yl)-N-(4-(methylcarbamoyl)benzyl)-1-(2-(prop-2-yn-1-yloxy)ethyl)-4,5,6,7-tetrahydro-1H-pyrazolo[4,3-c]pyridine-3-carboxamide (81 mg, 66% yield) as a white solid. (LC/MS) m/z (M+H)⁺=567.5. ¹H NMR (400 MHz, (CD₃)₂SO) δ 8.67 (t, 1H), 8.45 (d, 1H), 8.37 (q, 1H), 7.87 (dd, 1H), 7.81-7.74 (m, 2H), 7.42-7.34 (m, 3H), 7.18 (s, 1H), 7.07-6.98 (m, 1H), 5.11 (d, 1H), 4.53-4.40 (m, 3H), 4.34-4.20 (m, 3H), 4.18-4.11 (m, 2H), 3.94-3.86 (m, 2H), 3.76 (s, 3H), 3.50-3.42 (m, 1H), 3.41-3.32 (m, 2H), 2.77 (d, 3H), 1.24 (t, 3H).

### Step 4. Preparation of (S)-1-(2-((3-(3-(2,4-dioxotetrahydropyrimidin-1(2H)-yl)-1-methyl-1H-indazol-6-yl)prop-2-yn-1-yl)oxy)ethyl)-7-methyl-5-(5-(1-methyl-1H-imidazol-2-yl)pyridin-2-yl)-N-(4-(methylcarbamoyl)benzyl)-4,5,6,7-tetrahydro-1H-pyrazolo[4,3-c]pyridine-3-carboxamide (E733)

The same reaction was conducted in two batches then combined for purification. To a mixture of (S)-7-methyl-5-(5-(1-methyl-1H-imidazol-2-yl)pyridin-2-yl)-N-(4-(methylcarbamoyl)benzyl)-1-(2-(prop-2-yn-1-yloxy)ethyl)-4,5,6,7-tetrahydro-1H-pyrazolo[4,3-c]pyridine-3-carboxamide (0.060 g, 0.11 mmol) and 1-(6-bromo-1-methyl-1H-indazol-3-yl)dihydropyrimidine-2,4(1H,3H)-dione (CAS: 2446913-88-6; 38 mg, 0.12 mmol) in DMSO (0.6 mL) were added CuI (4.0 mg, 0.021 mmol), DIEA (55 mg, 0.42 mmol), and PdCl₂(PPh₃)₂(15 mg, 0.021 mmol) to form the first batch. The reaction mixture of the first batch was degassed with nitrogen gas for 3 min and then stirred at 90 °C for 7 h.

A second batch of the same reaction was conducted with (S)-7-methyl-5-(5-(1-methyl-1H-imidazol-2-yl)pyridin-2-yl)-N-(4-(methylcarbamoyl)benzyl)-1-(2-(prop-2-yn-1-yloxy)ethyl)-4,5,6,7-tetrahydro-1H-pyrazolo[4,3-c]pyridine-3-carboxamide (0.021 g, 0.037 mmol). The two batches were combined then was filtered. The filtrate was purified by reverse phase HPLC (Welch Xtimate C₁₈ 150 mm×30 mm×5 μm column; Mobile Phase A: H₂O (10 mM NH₄HCO₃)/Mobile Phase B: ACN; gradient of 26-66% of Mobile Phase B over 10 min; flow rate: 30 mL/min) and lyophilized to form (S)-1-(2-((3-(3-(2,4-dioxotetrahydropyrimidin-1(2H)-yl)-1-methyl-1H-indazol-6-yl)prop-2-yn-1-yl)oxy)ethyl)-7-methyl-5-(5-(1-methyl-1H-imidazol-2-yl)pyridin-2-yl)-N-(4-(methylcarbamoyl)benzyl)-4,5,6,7-tetrahydro-1H-pyrazolo[4,3-c]pyridine-3-carboxamide (2.4 mg, 2.1% yield) as a white solid. (LC/MS) m/z (M+H)⁺=809.4. ¹H NMR (400 MHz, (CD₃)₂SO) δ 10.61 (s, 1H), 8.71-8.67 (m, 1H), 8.44-8.34 (m, 2H), 7.91-7.72 (m, 4H), 7.64 (d, 1H), 7.36 (d, 2H), 7.22-7.18 (m, 1H), 7.14-7.07 (m, 1H), 6.95-6.91 (m, 2H), 5.06 (d, 1H), 4.46-4.20 (m, 8H), 4.04-3.96 (m, 5H), 3.92 (t, 2H), 3.70 (s, 3H), 3.31-3.28 (m, 2H), 2.75 (t, 5H), 1.26 (d, 3H).

### Examples E5-E8, E12-E25, E27-E90, E92-E214, E216-E229, E231-E247, E249-E272, E274-E284, E286-E460, E462-E510, E512-E528, E530-E544, E546-E617, E620-E709, E711-E732

The compounds in Table 3 and Table 4 were prepared using similar chemistry from Examples above that are commercially available or that can be synthesized by literature methods to form the resultant compounds of the disclosure.

Lengthy table referenced here
US20260345786A1-20261105-T00001
Please refer to the end of the specification for access instructions.

Lengthy table referenced here
US20260345786A1-20261105-T00002
Please refer to the end of the specification for access instructions.

Example E1^{a}: altered the amide coupling conditions; To a solution of carboxylic acid derivative (1.0 equivalent) in DMSO (0.07 M) was added DIEA (5.0 equivalents), EDCI (2.4 equivalents) and HOPO (3.0 equivalents). The reaction mixture was stirred at 40 °C for 30 min then the amine derivative (1.0 equivalent) was added. The suspension was stirred at 40 °C for 30 min then purified by reverse phase HPLC to provide the target product.

Example E2^{a}: altered the reductive amination conditions; A reaction mixture of the aldehyde derivative (1.0 equivalent), amine derivative (1.0 equivalent), AcOK (2.0 equivalents) and AcOH (2.0 equivalents) in MeOH (0.08 M) and DMSO (0.08 M) was stirred at room temperature for 20 minutes before NaBH(OAc)₃ (3.0 equivalents) was added. The reaction mixture was stirred for 16 h then purified by reverse phase HPLC to provide the target product.

Example E2^{b}: altered the final step from a reductive amination to a SNAr; To a solution of the mesylate derivative (1.2 equivalents) and the amine derivative (1.0 equivalent) in DMF (0.1M) was added DIEA (3.0 equivalents) and KI (0.1 equivalent). The reaction mixture was stirred at 90 °C for 16 h then filtered then concentrated in vacuo. The residue was purified by reverse phase HPLC to provide the target product.

Example E4^{b} and E11^{b}: altered the palladium coupling conditions; A reaction mixture of ethyl (S)-5-methyl-3-(trifluoromethyl)-5,6,7,8-tetrahydroimidazo[1,5-a]pyrazine-1-carboxylate (1 equivalent), tert-butyl 4-bromobenzoate (1.3 equivalents), P(t-Bu)₃ Pd G2 (0.05 equivalent) and KOTMS (3.5 equivalents) in tert-amyl alcohol (0.20 M) was degassed with nitrogen gas for 3 minutes then stirred at 100 °C for 1 h to provide (S)-7-(4-(tert-butoxycarbonyl)phenyl)-5-methyl-3-(trifluoromethyl)-5,6,7,8-tetrahydroimidazo[1,5-a]pyrazine-1-carboxylic acid (crude).

Example E4^{c}, E11^{c} and E230^{c}: altered the amide coupling conditions; A solution of the carboxylic acid derivative (1.0 equivalent), HOBt (1.5 equivalents), EDCI (1.5 equivalents), DIEA (3.0 equivalents) in ACN (0.1 M) was added to the amine derivative (1.0 equivalent) then stirred at room temperature overnight. The reaction was concentrated by genevac then the residue was diluted with DMSO and purified by reverse phase HPLC to provide the target product or intermediate.

Example E4^{d}: altered the deprotection conditions: HCl was used instead of TFA.

Example E4^{e}: SNAr altered: A reaction mixture of amine (1.0 equivalent), chloro-substituted heterocycle (1.1 equivalents), K₂CO₃ (3.0 equivalents) in DMSO (0.47M) was stirred at 50 °C for 20 min. The suspension was diluted with EtOAc and H₂O then the pH was adjusted to pH=7 with HCl or aqueous NaHCO₃. The organic layer was concentrated in vacuo to give the desired intermediate (crude).

Example E10^{a}: target product isolated as one of the by-products during purification.

Example E11^{a}: altered the protecting group to benzyloxycarbonyl (Cbz); therefore, the deprotection from step 2 was altered: To a solution of the Cbz-protected amine derivative (1.0 equivalent) in MeOH (0.03 M) was added 10% Pd/C (1.2 equivalents) at room temperature under argon gas. The reaction mixture was stirred at 50 °C for 3 h under H₂ gas at 45 psi then filtered. The filtrate was concentrated in vacuo to give the target intermediate.

Example E11^{d}: altered the deprotection conditions: TFA was used instead of HCl.

Example E230^{a}: altered the deprotection conditions: HCl was used instead of TFA.

Example E230^{d}: altered the palladium coupling conditions: To the solution of carboxylic acid derivative (1.0 equivalent) in DMSO (0.5 M) was added K₂CO₃ (3.0 equivalents) then the chloride derivative (1.1 equivalents). The reaction mixture was stirred at 50 °C for 18 min then cooled to room temperature. The suspension was diluted with EtOAc and H₂O then adjusted to pH-7 with HCl or aqueous NaHCO₃. The organic layer was separated then concentrated in vacuo to provide the desired intermediate (crude).

Example E230^{e}: altered the palladium coupling conditions: To a mixture of rel-ethyl (R)-1-ethyl-7-(trifluoromethyl)-4,5,6,7-tetrahydro-1H-pyrazolo[4,3-c]pyridine-3-carboxylate hydrochloride (0.700 g, 2.14 mmol) in dioxane (2 mL) was added K₃PO₄ (1.36 g, 0.53 mL, 6.41 mmol), tert-butyl 6-bromonicotinate (662 mg, 2.56 mmol), and RuPhos Pd G4 (182 mg, 0.214 mmol). The reaction was degassed with nitrogen gas then tert-amyl alcohol (10 mL) was added. The suspension was stirred at 100 °C for 1 h then another portion of RuPhos Pd G4 (182 mg, 0.214 mmol) was added. The reaction was stirred at 100 °C for another hour then stirred at 85 °C overnight. The reaction was diluted with DCM and filtered. The filtrate was concentrated by reduced pressure then dissolved in DCM and purified by column chromatography (silica gel; 10-100% EtOAc: heptane) then column chromatography (silica gel; 5-20% MeOH: DCM) to give the desired ester intermediate, which transformed further under hydrolysis conditions such as with a base like NaOH (4 equivalents) in a solvent such as THF (0.2M) at 45 °C to give the desired acid intermediate.

Example 485^{a}: An additional amide coupling at the last step of the synthesis.

### Chiral Purification Data:

Example E171 and E172\hat{ }: A mixture was purified using SFC [Column: DAICEL CHIRALPAK ID 250 mm×30 mm×10 μm; Mobile phase A: CO₂/Mobile Phase B: MeOH; isocratic at 50% of Mobile Phase B; flow rate: 100 mL/min; column temperature: 40 °C; back pressure: 100 bar]. The chiral purity was determined using chiral HPLC analytical method on Chiralpak ID-3 100 mm×4.6 mm×3 μm column; Mobile phase A: MeOH (0.05% DEA); Mobile phase B: ACN; isocratic at 50% of Mobile Phase B; flow rate: 2.0 mL/min; column temperature: 35 °C to provide Example E171: peak 1 (retention time: 2.240 min, 98% ee) and Example E172: peak 2 (retention time: 2.784 min, 96% ee).

Example E503 and E504\hat{ }: A E513 (1.73 g, 1.75 mmol) was purified using reverse phase HPLC [Chiral Tech ID 250 mm×21.2 mm×5 μm column; Mobile phase A: MeOH (10 mM ammonium formate)/Mobile Phase B: ACN; isocratic at 50% of Mobile Phase B; flow rate: 35 mL/min to provide peak 1 (3.45 g) and peak 2 (4.60 g).

Peak 1 (3.45 g, 3.49 mmol) was treated with methyl ethyl ketone (60 mL), 0.05 M pH=7 phosphate buffer (10 mL), brine (10 mL) and saturated NaHCO₃ (6 mL) to pH=7. To the mixture was added DCM (20 mL). The separated organic phase was washed with brine (20 mL)/and pH=7 phosphate buffer (10 mL). The aqueous phase was extracted with DCM (50 mL). The combined organic phase was dried by Na₂SO₄, concentrated in vacuo then dried by high vacuum to give E504 (0.818 g, 47.3% yield) as a glass.

Peak 2 (4.60 g, 4.65 mmol) was treated with DCM (60 mL), 0.05 M pH=7 phosphate buffer (10 mL), brine (10 mL) and saturated NaHCO₃ (8 mL) to pH=7. The separated organic phase was washed with brine (20 mL)/and pH=7 buffer (10 mL). The aqueous phase was extracted with DCM (50 mL) sequentially. The combined organic phase was dried by Na₂SO₄, concentrated in vacuo then dried by high vacuum to give E503 (0.737 g, 42.1% yield) as a glass.

The chiral purity was determined using HPLC analytical method on Chiral Tech ID 50 mm×4.6 mm×5 μm column; Mobile phase A: MeOH (10 mM ammonium formate); Mobile phase B: ACN; isocratic at 50% of Mobile Phase B; flow rate: 2.0 mL/min; run time: 6 min to provide Example E504: peak 1 (retention time: 2.606 min, 93% ee) and Example E503: peak 2 (retention time: 3.492 min, 89% ee).

Example E558\hat{ } and E651\hat{ }: A mixture of regioisomers were purified using SFC [Column: ChiralPak IH 250 mm×30 mm×10 μm; Mobile phase A: CO₂/Mobile Phase B: (4:1, IPA: ACN) with 0.1% NH₄OH; isocratic at 70% of Mobile Phase B; flow rate: 80 mL/min; column temperature: 40 °C; back pressure: 100 bar]. The chiral purity was determined using chiral HPLC analytical method on Chiralpak IH 100 mm×4.6 mm×3 μm column; Mobile phase A: hexane (0.1% DEA); Mobile phase B: (2:1, IPA: ACN); isocratic at 60% of Mobile Phase B; flow rate: 1.0 mL/min; column temperature: 35 °C to provide Example E651: peak 1 (retention time: 4.145 min, 100% ee) and Example E558: peak 2 (retention time: 6.210 min, 99% ee). Example E565\hat{ }: A mixture was purified using SFC [Column: DAICEL CHIRALPAK IK 250 mm×25 mm×10 μm; Mobile phase A: Heptane/Mobile Phase B: (4:1, IPA: ACN) with 0.1% NH₄OH; isocratic at 100% of Mobile Phase B; flow rate: 80 mL/min]. The chiral purity was determined using chiral HPLC analytical method on Chiralpak IK 50 mm×4.6 mm×3 μm column; Mobile phase A: hexane (0.1% IDEA); Mobile phase B: (2:1, IPA: ACN); isocratic at 80% of Mobile Phase B; flow rate: 1 0.0 mL/min; column temperature: 35 °C to provide peak 1 (retention time: 4.506 min, 97% ee) and Example E565: peak 2 (retention time: 5.593 min, 98% ee).

### Prophetic Examples

The compounds in Table 5 can be prepared using similar chemistry from the examples above that are commercially available or that can be synthesized by literature methods to form the resultant compounds of the disclosure. It is noted that where the stereochemistry of the compounds in Table 5 is not defined, the respective bonds can include S or R stereoisomers or can be a racemic mixture thereof.

TABLE 5

| Example | | |
|---|---|---|
| number | Structure | ChemDraw Name |
| VE7 | 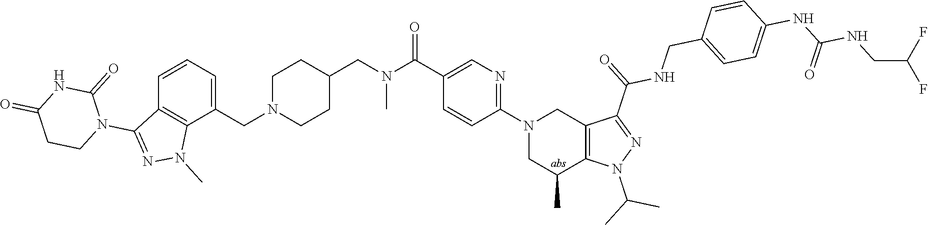 [SMILES: CC(C)n1nc(C(=O)NCc2ccc(NC(=O)NCC(F)F)cc2)c2c1[C@@H](C)CN(c1ccc(C(=O)N(C)CC3CCN(Cc4cccc5c(N6CCC(=O)NC6=O)nn(C)c45)CC3)cn1)C2] | (S)-N-(4-(3-(2,2- difluoroethyl)ureido)benzyl)-5-(5- (((1-((3-(2,4- dioxotetrahydropyrimidin-1(2H)- yl)-1-methyl-1H-indazol-7- yl)methyl)piperidin-4- yl)methyl)(methyl)carbamoyl) pyridin-2-yl)-1-isopropyl-7-methyl- 4,5,6,7-tetrahydro-1H- pyrazolo[4,3-c]pyridine-3- carboxamide |
| VE8 | 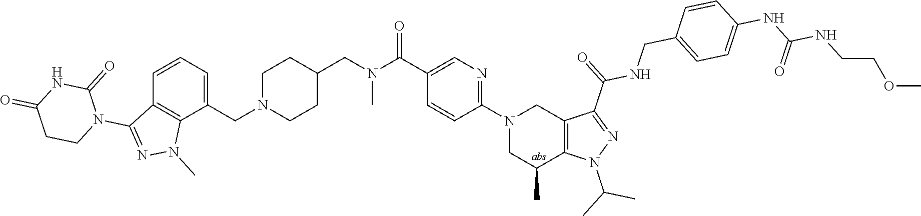 [SMILES: COCCNC(=O)Nc1ccc(CNC(=O)c2nn(C(C)C)c3c2CN(c2ccc(C(=O)N(C)CC4CCN(Cc5cccc6c(N7CCC(=O)NC7=O)nn(C)c56)CC4)cn2)C[C@@H]3C)cc1] | (S)-5-(5-(((1-((3-(2,4- dioxotetrahydropyrimidin-1(2H)- yl)-1-methyl-1H-indazol-7- yl)methyl)piperidin-4- yl)methyl)(methyl)carbamoyl) pyridin-2-yl)-1-isopropyl-N-(4-(3-(2- methoxyethyl)ureido)benzyl)-7- methyl-4,5,6,7-tetrahydro-1H- pyrazolo[4,3-c]pyridine-3- carboxamide |
| VE9 | 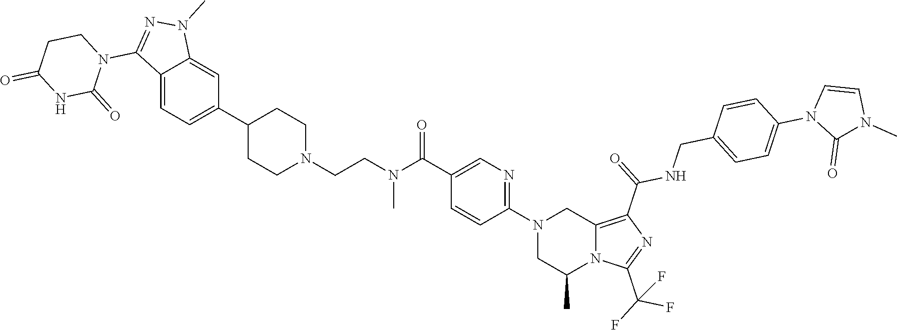 [SMILES: C[C@H]1CN(c2ccc(C(=O)N(C)CCN3CCC(c4ccc5c(N6CCC(=O)NC6=O)nn(C)c5c4)CC3)cn2)Cc2c(C(=O)NCc3ccc(-n4ccn(C)c4=O)cc3)nc(C(F)(F)F)n21] | (S)-7-(5-((2-(4-(3-(2,4- dioxotetrahydropyrimidin-1(2H)- yl)-1-methyl-1H-indazol-6- yl)piperidin-1- yl)ethyl)(methyl)carbamoyl)pyridin- 2-yl)-5-methyl-N-(4-(3-methyl- 2-oxo-2,3-dihydro-1H-imidazol- 1-yl)benzyl)-3-(trifluoromethyl)- 5,6,7,8-tetrahydroimidazo[1,5- a]pyrazine-1-carboxamide |
| VE10 | 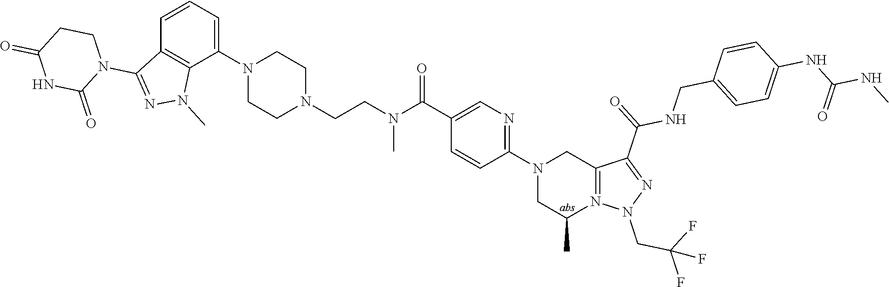 | (S)-5-(5-((2-(4-(3-(2,4- dioxotetrahydropyrimidin-1(2H)- yl)-1-methyl-1H-indazol-7- yl)piperazin-1- yl)ethyl)(methyl)carbamoyl)pyridin- 2-yl)-7-methyl-N-(4-(3- methylureido)benzyl)-1-(2,2,2- trifluoroethyl)-4,5,6,7-tetrahydro- 1H-pyrazolo[4,3-c]pyridine-3- carboxamide |
| VE19 | 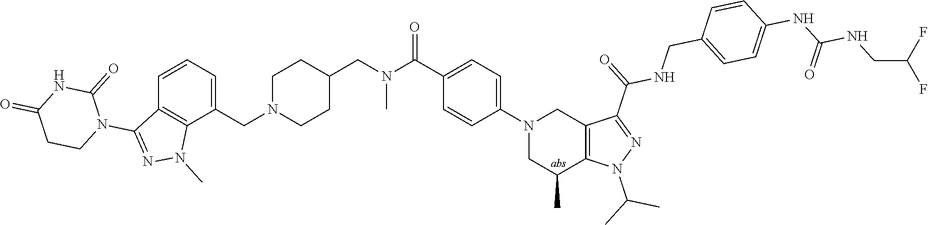 [SMILES: CC(C)n1nc(C(=O)NCc2ccc(NC(=O)NCC(F)F)cc2)c2c1[C@@H](C)CN(c1ccc(C(=O)N(C)CC3CCN(Cc4cccc5c(N6CCC(=O)NC6=O)nn(C)c45)CC3)cc1)C2] | (S)-N-(4-(3-(2,2- difluoroethyl)ureido)benzyl)-5-(4- (((1-((3-(2,4- dioxotetrahydropyrimidin-1(2H)- yl)-1-methyl-1H-indazol-7- yl)methyl)piperidin-4- yl)methyl)(methyl)carbamoyl) phenyl)-1-isopropyl-7-methyl- 4,5,6,7-tetrahydro-1H- pyrazolo[4,3-c]pyridine-3- carboxamide |
| VE20 | 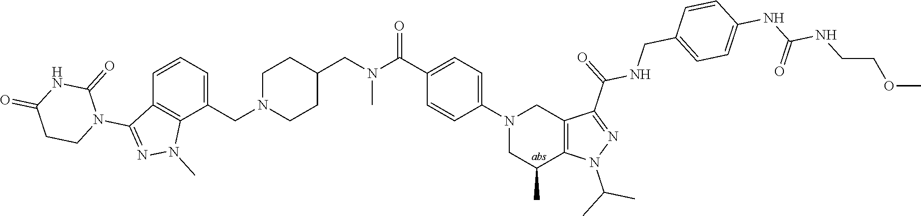 [SMILES: COCCNC(=O)Nc1ccc(CNC(=O)c2nn(C(C)C)c3c2CN(c2ccc(C(=O)N(C)CC4CCN(Cc5cccc6c(N7CCC(=O)NC7=O)nn(C)c56)CC4)cc2)C[C@@H]3C)cc1] | (S)-5-(4-(((1-((3-(2,4- dioxotetrahydropyrimidin-1(2H)- yl)-1-methyl-1H-indazol-7- yl)methyl)piperidin-4- yl)methyl)(methyl)carbamoyl) phenyl)-1-isopropyl-N-(4-(3-(2- methoxyethyl)ureido)benzyl)-7- methyl-4,5,6,7-tetrahydro-1H- pyrazolo[4,3-c]pyridine-3- carboxamide |
| VE28 | 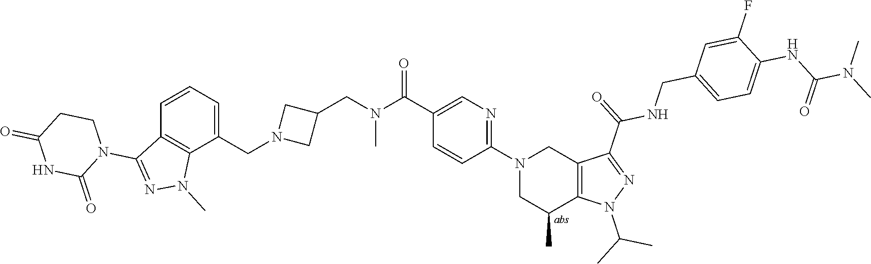 [SMILES: CC(C)n1nc(C(=O)NCc2ccc(NC(=O)N(C)C)c(F)c2)c2c1[C@@H](C)CN(c1ccc(C(=O)N(C)CC3CN(Cc4cccc5c(N6CCC(=O)NC6=O)nn(C)c45)C3)cn1)C2] | (S)-N-(4-(3,3-dimethylureido)-3- fluorobenzyl)-5-(5-(((1-((3-(2,4- dioxotetrahydropyrimidin-1(2H)- yl)-1-methyl-1H-indazol-7- yl)methyl)azetidin-3- yl)methyl)(methyl)carbamoyl)pyridin- 2-yl)-1-isopropyl-7-methyl- 4,5,6,7-tetrahydro-1H- pyrazolo[4,3-c]pyridine-3- carboxamide |
| VE37 | 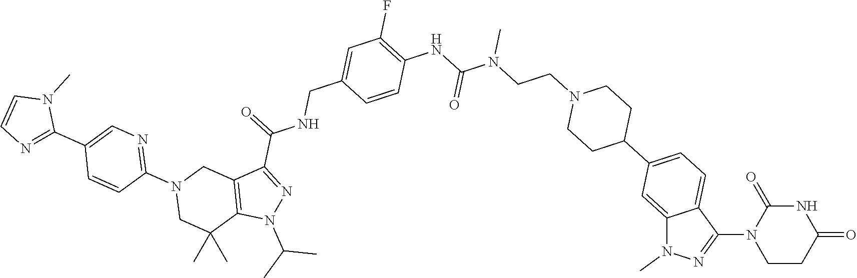 [SMILES: CC(C)n1nc(C(=O)NCc2ccc(NC(=O)N(C)CCN3CCC(c4ccc5c(N6CCC(=O)NC6=O)nn(C)c5c4)CC3)c(F)c2)c2c1C(C)(C)CN(c1ccc(-c3nccn3C)cn1)C2] | N-(4-(3-(2-(4-(3-(2,4- dioxotetrahydropyrimidin-1(2H)- yl)-1-methyl-1H-indazol-6- yl)piperidin-1-yl)ethyl)-3- methylureido)-3-fluorobenzyl)-1- isopropyl-7,7-dimethyl-5-(5-(1- methyl-1H-imidazol-2-yl)pyridin- 2-yl)-4,5,6,7-tetrahydro-1H- pyrazolo[4,3-c]pyridine-3- carboxamide |
| VE38 | 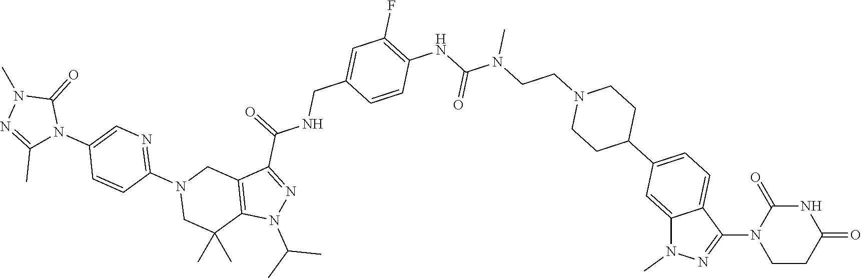 [SMILES: Cc1nn(C)c(=O)n1-c1ccc(N2Cc3c(C(=O)NCc4ccc(NC(=O)N(C)CCN5CCC(c6ccc7c(N8CCC(=O)NC8=O)nn(C)c7c6)CC5)c(F)c4)nn(C(C)C)c3C(C)(C)C2)nc1] | 5-(5-(1,3-dimethyl-5-oxo-1,5- dihydro-4H-1,2,4-triazol-4- yl)pyridin-2-yl)-N-(4-(3-(2-(4-(3- (2,4-dioxotetrahydropyrimidin- 1(2H)-yl)-1-methyl-1H-indazol-6- yl)piperidin-1-yl)ethyl)-3- methylureido)-3-fluorobenzyl)-1- isopropyl-7,7-dimethyl-4,5,6,7- tetrahydro-1H-pyrazolo[4,3- c]pyridine-3-carboxamide |
| VE71 | 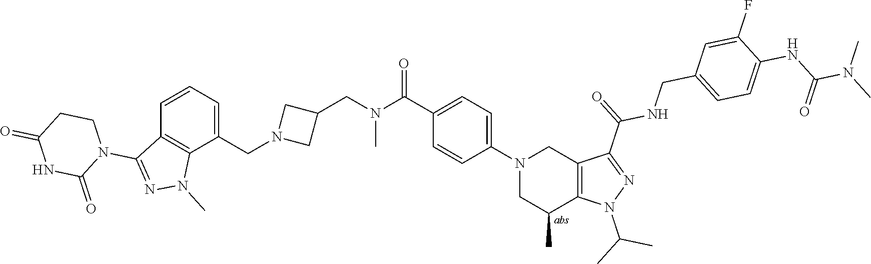 [SMILES: CC(C)n1nc(C(=O)NCc2ccc(NC(=O)N(C)C)c(F)c2)c2c1[C@@H](C)CN(c1ccc(C(=O)N(C)CC3CN(Cc4cccc5c(N6CCC(=O)NC6=O)nn(C)c45)C3)cc1)C2] | (S)-N-(4-(3,3-dimethylureido)-3- fluorobenzyl)-5-(4-(((1-((3-(2,4- dioxotetrahydropyrimidin-1(2H)- yl)-1-methyl-1H-indazol-7- yl)methyl)azetidin-3- yl)methyl)(methyl)carbamoyl) phenyl)-1-isopropyl-7-methyl- 4,5,6,7-tetrahydro-1H- pyrazolo[4,3-c]pyridine-3- carboxamide |
| VE85 | 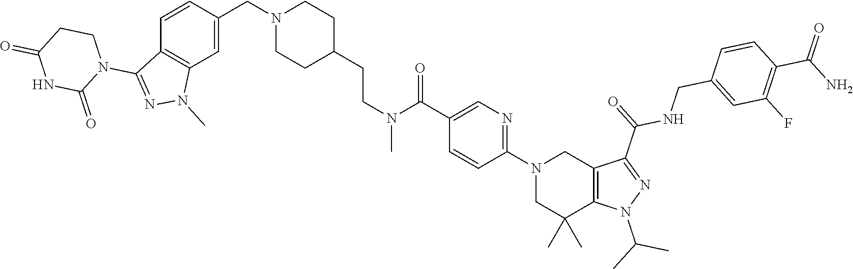 [SMILES: CC(C)n1nc(C(=O)NCc2ccc(C(N)=O)c(F)c2)c2c1C(C)(C)CN(c1ccc(C(=O)N(C)CCC3CCN(Cc4ccc5c(N6CCC(=O)NC6=O)nn(C)c5c4)CC3)cn1)C2] | N-(4-carbamoyl-3-fluorobenzyl)- 5-(5-((2-(1-((3-(2,4- dioxotetrahydropyrimidin-1(2H)- yl)-1-methyl-1H-indazol-6- yl)methyl)piperidin-4- yl)ethyl)(methyl)carbamoyl)pyridin- 2-yl)-1-isopropyl-7,7-dimethyl- 4,5,6,7-tetrahydro-1H- pyrazolo[4,3-c]pyridine-3- carboxamide |
| VE89 | 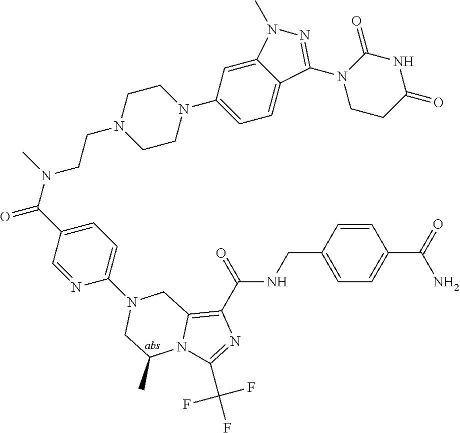 [SMILES: C[C@H]1CN(c2ccc(C(=O)N(C)CCN3CCN(c4ccc5c(N6CCC(=O)NC6=O)nn(C)c5c4)CC3)cn2)Cc2c(C(=O)NCc3ccc(C(N)=O)cc3)nc(C(F)(F)F)n21] | (S)-N-(4-carbamoylbenzyl)-7-(5- ((2-(4-(3-(2,4- dioxotetrahydropyrimidin-1(2H)- yl)-1-methyl-1H-indazol-6- yl)piperazin-1- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE90 | 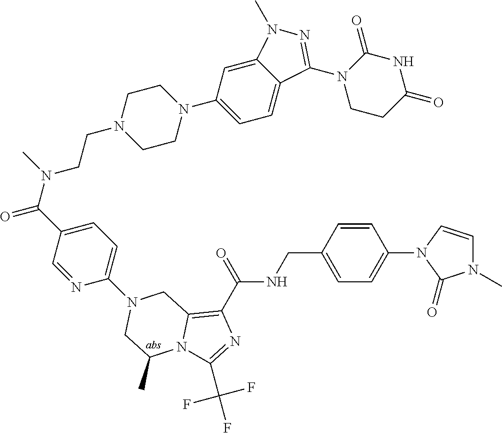 [SMILES: C[C@H]1CN(c2ccc(C(=O)N(C)CCN3CCN(c4ccc5c(N6CCC(=O)NC6=O)nn(C)c5c4)CC3)cn2)Cc2c(C(=O)NCc3ccc(-n4ccn(C)c4=O)cc3)nc(C(F)(F)F)n21] | (S)-7-(5-((2-(4-(3-(2,4- dioxotetrahydropyrimidin-1(2H)- yl)-1-methyl-1H-indazol-6- yl)piperazin-1- yl)ethyl)(methyl)carbamoyl)pyridin- 2-yl)-5-methyl-N-(4-(3-methyl- 2-oxo-2,3-dihydro-1H-imidazol- 1-yl)benzyl)-3-(trifluoromethyl)- 5,6,7,8-tetrahydroimidazo[1,5- a]pyrazine-1-carboxamide |
| VE91 | 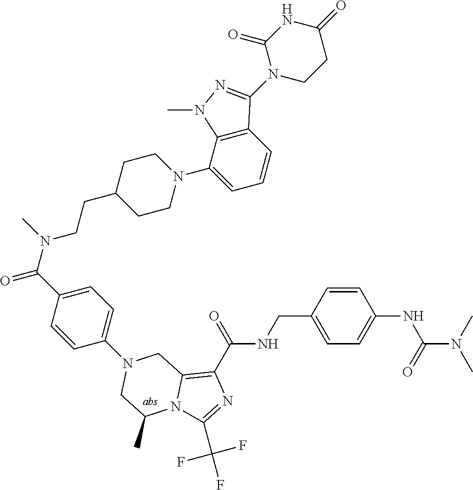 [SMILES: C[C@H]1CN(c2ccc(C(=O)N(C)CCC3CCN(c4cccc5c(N6CCC(=O)NC6=O)nn(C)c45)CC3)cc2)Cc2c(C(=O)NCc3ccc(NC(=O)N(C)C)cc3)nc(C(F)(F)F)n21] | (S)-N-(4-(3,3- dimethylureido)benzyl)-7-(4-((2- (1-(3-(2,4- dioxotetrahydropyrimidin-1(2H)- yl)-1-methyl-1H-indazol-7- yl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE92 | 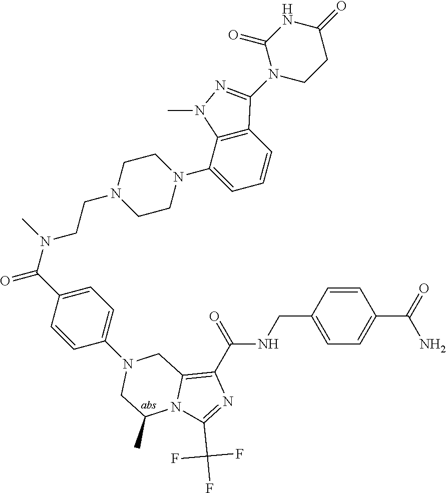 [SMILES: C[C@H]1CN(c2ccc(C(=O)N(C)CCN3CCN(c4cccc5c(N6CCC(=O)NC6=O)nn(C)c45)CC3)cc2)Cc2c(C(=O)NCc3ccc(C(N)=O)cc3)nc(C(F)(F)F)n21] | (S)-N-(4-carbamoylbenzyl)-7-(4- ((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE93 | 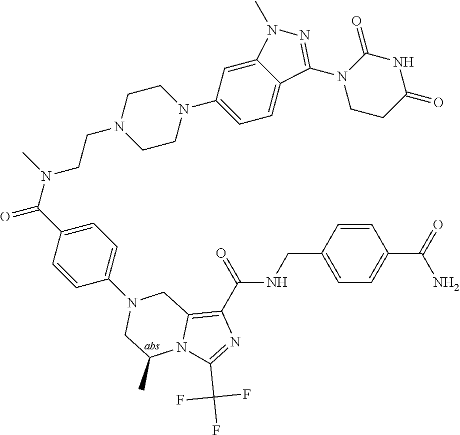 [SMILES: C[C@H]1CN(c2ccc(C(=O)N(C)CCN3CCN(c4ccc5c(N6CCC(=O)NC6=O)nn(C)c5c4)CC3)cc2)Cc2c(C(=O)NCc3ccc(C(N)=O)cc3)nc(C(F)(F)F)n21] | (S)-N-(4-carbamoylbenzyl)-7-(4- ((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE94 | 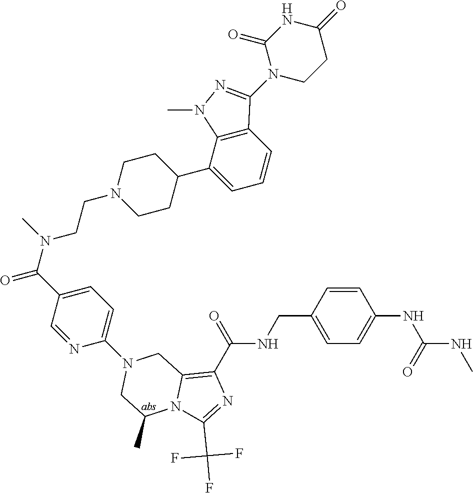 [SMILES: CNC(=O)Nc1ccc(CNC(=O)c2nc(C(F)(F)F)n3c2CN(c2ccc(C(=O)N(C)CCN4CCC(c5cccc6c(N7CCC(=O)NC7=O)nn(C)c56)CC4)cn2)C[C@@H]3C)cc1] | (S)-7-(5-((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-5-methyl-N-(4-(3- methylureido)benzyl)-3- (trifluoromethyl)-5,6,7,8- tetrahydroimidazo[1,5- a]pyrazine-1-carboxamide |
| VE95 | 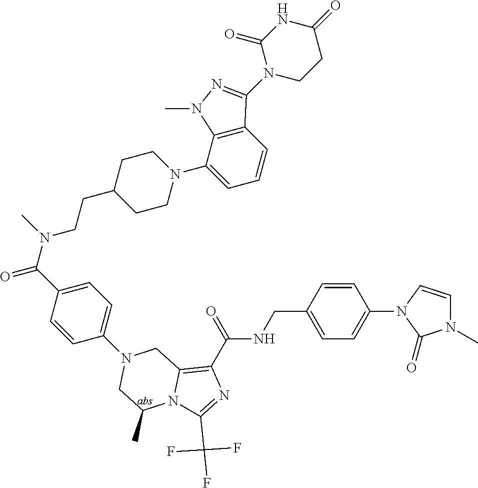 [SMILES: C[C@H]1CN(c2ccc(C(=O)N(C)CCC3CCN(c4cccc5c(N6CCC(=O)NC6=O)nn(C)c45)CC3)cc2)Cc2c(C(=O)NCc3ccc(-n4ccn(C)c4=O)cc3)nc(C(F)(F)F)n21] | (S)-7-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE96 | 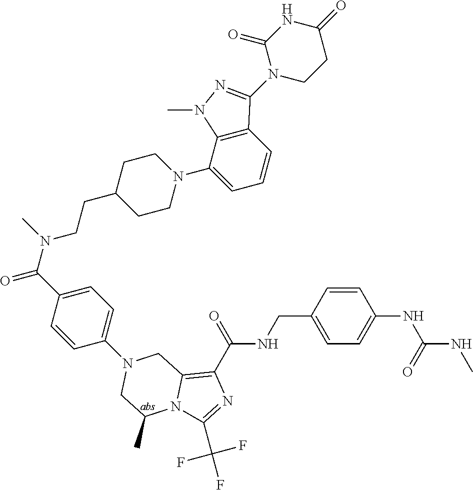 [SMILES: CNC(=O)Nc1ccc(CNC(=O)c2nc(C(F)(F)F)n3c2CN(c2ccc(C(=O)N(C)CCC4CCN(c5cccc6c(N7CCC(=O)NC7=O)nn(C)c56)CC4)cc2)C[C@@H]3C)cc1] | (S)-7-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 5-methyl-N-(4-(3- methylureido)benzyl)-3- (trifluoromethyl)-5,6,7,8- tetrahydroimidazo[1,5- a]pyrazine-1-carboxamide |
| VE97 | 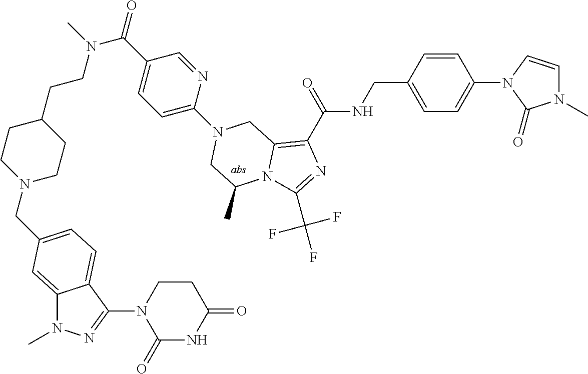 [SMILES: C[C@H]1CN(c2ccc(C(=O)N(C)CCC3CCN(Cc4ccc5c(N6CCC(=O)NC6=O)nn(C)c5c4)CC3)cn2)Cc2c(C(=O)NCc3ccc(-n4ccn(C)c4=O)cc3)nc(C(F)(F)F)n21] | (S)-7-(5-((2-(1-((3-(2,4- dioxotetrahydropyrimidin-1(2H)- yl)-1-methyl-1H-indazol-6- yl)methyl)piperidin-4- yl)ethyl)(methyl)carbamoyl)pyridin- 2-yl)-5-methyl-N-(4-(3-methyl- 2-oxo-2,3-dihydro-1H-imidazol- 1-yl)benzyl)-3-(trifluoromethyl)- 5,6,7,8-tetrahydroimidazo[1,5- a]pyrazine-1-carboxamide |
| VE99 | 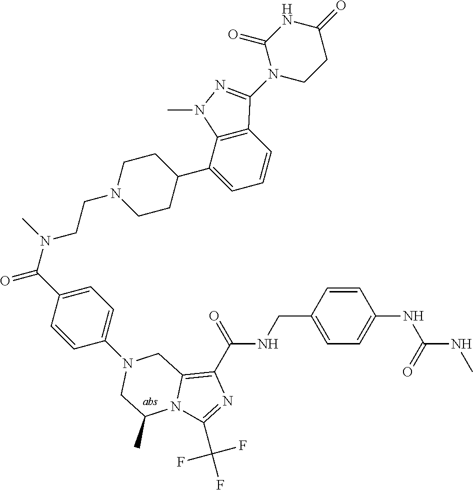 [SMILES: CNC(=O)Nc1ccc(CNC(=O)c2nc(C(F)(F)F)n3c2CN(c2ccc(C(=O)N(C)CCN4CCC(c5cccc6c(N7CCC(=O)NC7=O)nn(C)c56)CC4)cc2)C[C@@H]3C)cc1] | (S)-7-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 5-methyl-N-(4-(3- methylureido)benzyl)-3- (trifluoromethyl)-5,6,7,8- tetrahydroimidazo[1,5- a]pyrazine-1-carboxamide |
| VE100 | 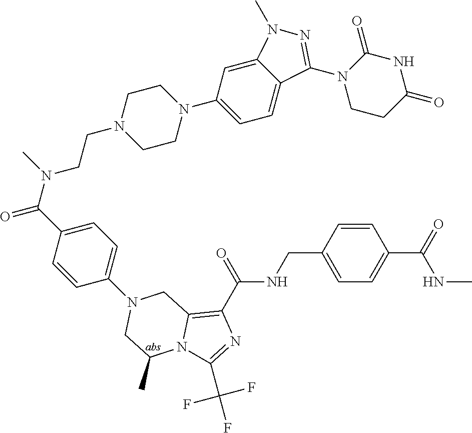 [SMILES: CNC(=O)c1ccc(CNC(=O)c2nc(C(F)(F)F)n3c2CN(c2ccc(C(=O)N(C)CCN4CCN(c5ccc6c(N7CCC(=O)NC7=O)nn(C)c6c5)CC4)cc2)C[C@@H]3C)cc1] | (S)-7-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 5-methyl-N-(4- (methylcarbamoyl)benzyl)-3- (trifluoromethyl)-5,6,7,8- tetrahydroimidazo[1,5- a]pyrazine-1-carboxamide |
| VE101 | 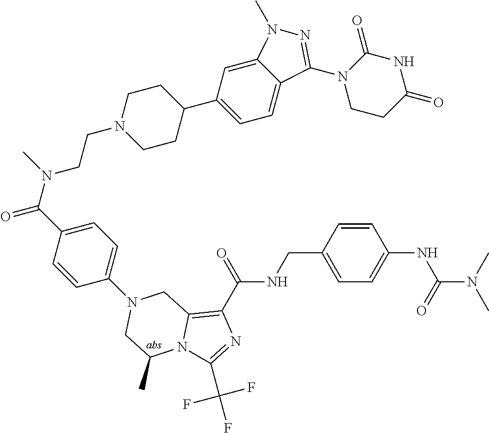 [SMILES: C[C@H]1CN(c2ccc(C(=O)N(C)CCN3CCC(c4ccc5c(N6CCC(=O)NC6=O)nn(C)c5c4)CC3)cc2)Cc2c(C(=O)NCc3ccc(NC(=O)N(C)C)cc3)nc(C(F)(F)F)n21] | (S)-N-(4-(3,3- dimethylureido)benzyl)-7-(4-((2- (4-(3-(2,4- dioxotetrahydropyrimidin-1(2H)- yl)-1-methyl-1H-indazol-6- yl)piperidin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE102 | 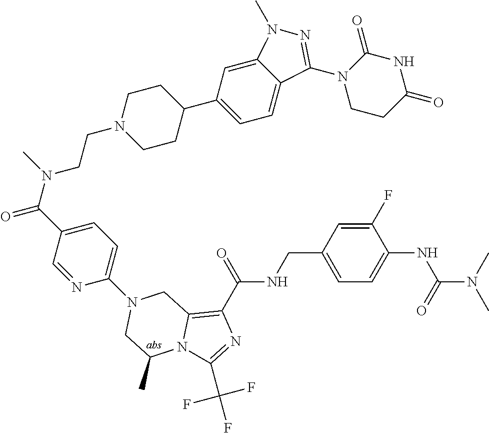 [SMILES: C[C@H]1CN(c2ccc(C(=O)N(C)CCN3CCC(c4ccc5c(N6CCC(=O)NC6=O)nn(C)c5c4)CC3)cn2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(5-((2-(1-(3-(2,4- dioxotetrahydropyrimidin-1(2H)- yl)-1-methyl-1H-indazol-6- yl)piperidin-4- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE103 | 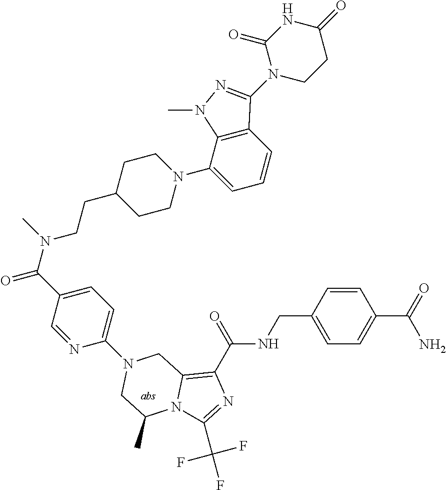 [SMILES: C[C@H]1CN(c2ccc(C(=O)N(C)CCC3CCN(c4cccc5c(N6CCC(=O)NC6=O)nn(C)c45)CC3)cn2)Cc2c(C(=O)NCc3ccc(C(N)=O)cc3)nc(C(F)(F)F)n21] | (S)-N-(4-carbamoylbenzyl)-7-(5- ((2-(1-(3-(2,4- dioxotetrahydropyrimidin-1(2H)- yl)-1-methyl-1H-indazol-7- yl)piperidin-4- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE104 | 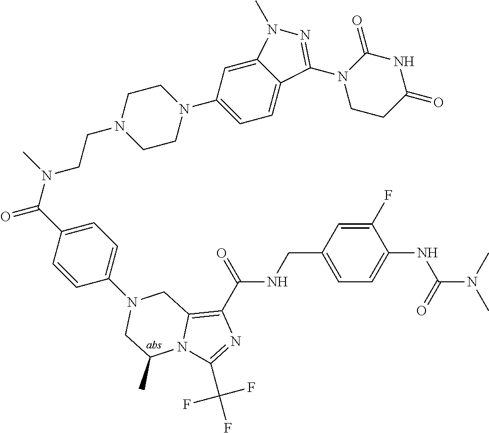 [SMILES: C[C@H]1CN(c2ccc(C(=O)N(C)CCN3CCN(c4ccc5c(N6CCC(=O)NC6=O)nn(C)c5c4)CC3)cc2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE105 | 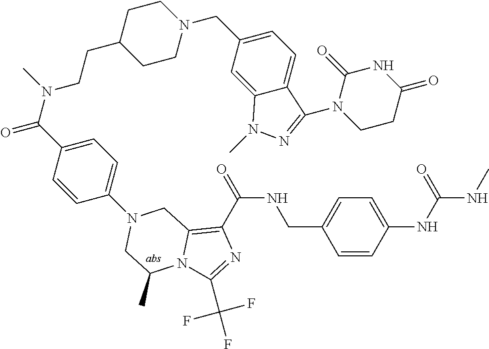 [SMILES: CNC(=O)Nc1ccc(CNC(=O)c2nc(C(F)(F)F)n3c2CN(c2ccc(C(=O)N(C)CCC4CCN(Cc5ccc6c(N7CCC(=O)NC7=O)nn(C)c6c5)CC4)cc2)C[C@@H]3C)cc1] | (S)-7-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 5-methyl-N-(4-(3- methylureido)benzyl)-3- (trifluoromethyl)-5,6,7,8- tetrahydroimidazo[1,5- a]pyrazine-1-carboxamide |
| VE106 | 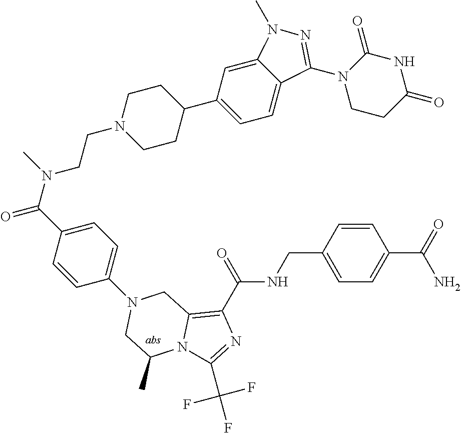 [SMILES: C[C@H]1CN(c2ccc(C(=O)N(C)CCN3CCC(c4ccc5c(N6CCC(=O)NC6=O)nn(C)c5c4)CC3)cc2)Cc2c(C(=O)NCc3ccc(C(N)=O)cc3)nc(C(F)(F)F)n21] | (S)-N-(4-carbamoylbenzyl)-7-(4- ((2-(4-(3-(2,4- dioxotetrahydropyrimidin-1(2H)- yl)-1-methyl-1H-indazol-6- yl)piperidin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE107 | 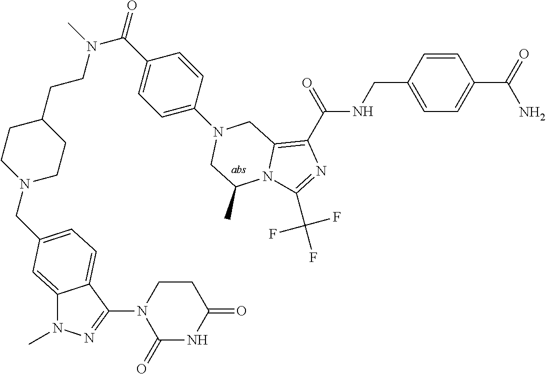 [SMILES: C[C@H]1CN(c2ccc(C(=O)N(C)CCC3CCN(Cc4ccc5c(N6CCC(=O)NC6=O)nn(C)c5c4)CC3)cc2)Cc2c(C(=O)NCc3ccc(C(N)=O)cc3)nc(C(F)(F)F)n21] | (S)-N-(4-carbamoylbenzyl)-7-(4- ((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE108 | 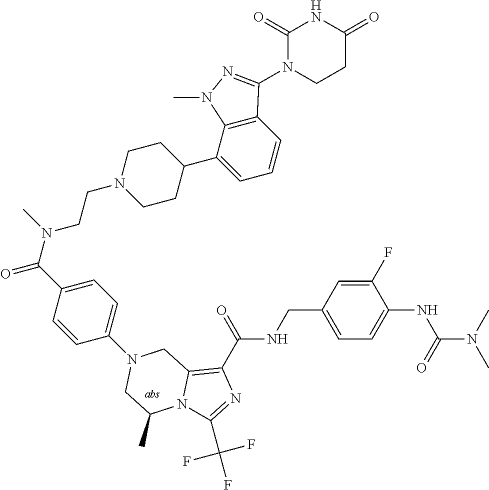 [SMILES: C[C@H]1CN(c2ccc(C(=O)N(C)CCN3CCC(c4cccc5c(N6CCC(=O)NC6=O)nn(C)c45)CC3)cc2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE109 | 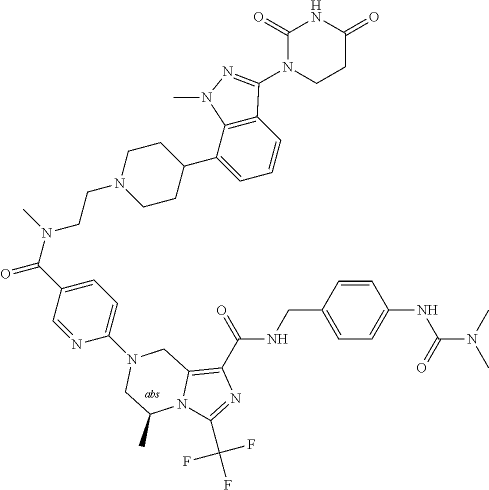 [SMILES: C[C@H]1CN(c2ccc(C(=O)N(C)CCN3CCC(c4cccc5c(N6CCC(=O)NC6=O)nn(C)c45)CC3)cn2)Cc2c(C(=O)NCc3ccc(NC(=O)N(C)C)cc3)nc(C(F)(F)F)n21] | (S)-N-(4-(3,3- dimethylureido)benzyl)-7-(5-((2- (4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE110 | 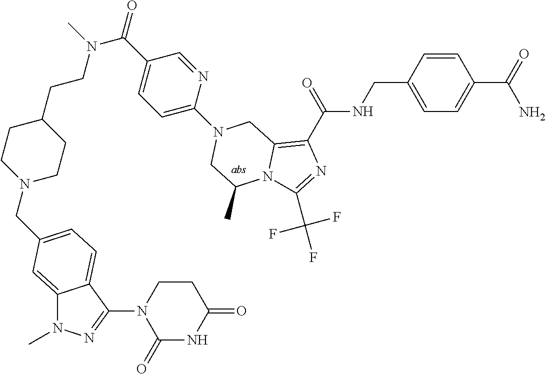 [SMILES: C[C@H]1CN(c2ccc(C(=O)N(C)CCC3CCN(Cc4ccc5c(N6CCC(=O)NC6=O)nn(C)c5c4)CC3)cn2)Cc2c(C(=O)NCc3ccc(C(N)=O)cc3)nc(C(F)(F)F)n21] | (S)-N-(4-carbamoylbenzyl)-7-(5- ((2-(1-((3-(2,4- dioxotetrahydropyrimidin-1(2H)- yl)-1-methyl-1H-indazol-6- yl)methyl)piperidin-4- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE111 | 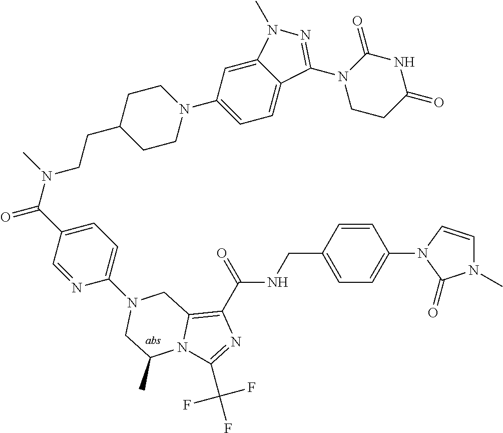 [SMILES: C[C@H]1CN(c2ccc(C(=O)N(C)CCC3CCN(c4ccc5c(N6CCC(=O)NC6=O)nn(C)c5c4)CC3)cn2)Cc2c(C(=O)NCc3ccc(-n4ccn(C)c4=O)cc3)nc(C(F)(F)F)n21] | (S)-7-(5-((2-(1-(3-(2,4- dioxotetrahydropyrimidin-1(2H)- yl)-1-methyl-1H-indazol-6- yl)piperidin-4- yl)ethyl)(methyl)carbamoyl)pyridin- 2-yl)-5-methyl-N-(4-(3-methyl- 2-oxo-2,3-dihydro-1H-imidazol- 1-yl)benzyl)-3-(trifluoromethyl)- 5,6,7,8-tetrahydroimidazo[1,5- a]pyrazine-1-carboxamide |
| VE112 | 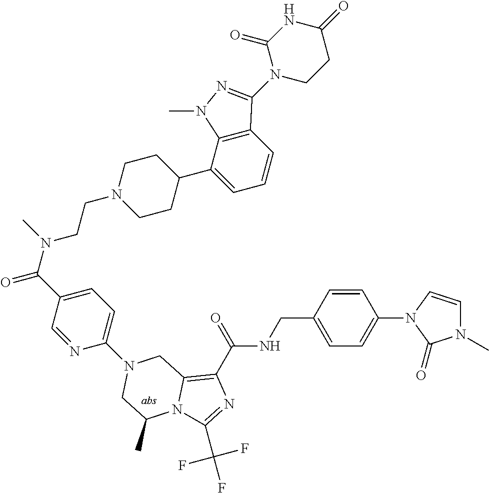 [SMILES: C[C@H]1CN(c2ccc(C(=O)N(C)CCN3CCC(c4cccc5c(N6CCC(=O)NC6=O)nn(C)c45)CC3)cn2)Cc2c(C(=O)NCc3ccc(-n4ccn(C)c4=O)cc3)nc(C(F)(F)F)n21] | (S)-7-(5-((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-5-methyl-N-(4-(3-methyl- 2-oxo-2,3-dihydro-1H-imidazol- 1-yl)benzyl)-3-(trifluoromethyl)- 5,6,7,8-tetrahydroimidazo[1,5- a]pyrazine-1-carboxamide |
| VE113 | 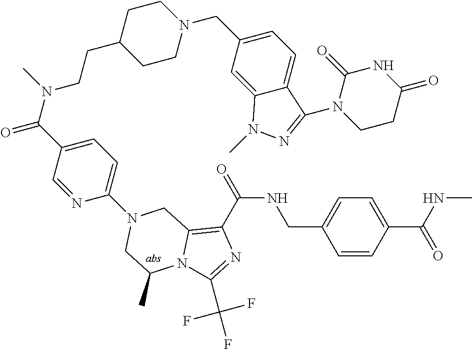 [SMILES: CNC(=O)c1ccc(CNC(=O)c2nc(C(F)(F)F)n3c2CN(c2ccc(C(=O)N(C)CCC4CCN(Cc5ccc6c(N7CCC(=O)NC7=O)nn(C)c6c5)CC4)cn2)C[C@@H]3C)cc1] | (S)-7-(5-((2-(1-((3-(2,4- dioxotetrahydropyrimidin-1(2H)- yl)-1-methyl-1H-indazol-6- yl)methyl)piperidin-4- yl)ethyl)(methyl)carbamoyl)pyridin- 2-yl)-5-methyl-N-(4- (methylcarbamoyl)benzyl)-3- (trifluoromethyl)-5,6,7,8- tetrahydroimidazo[1,5- a]pyrazine-1-carboxamide |
| VE114 | 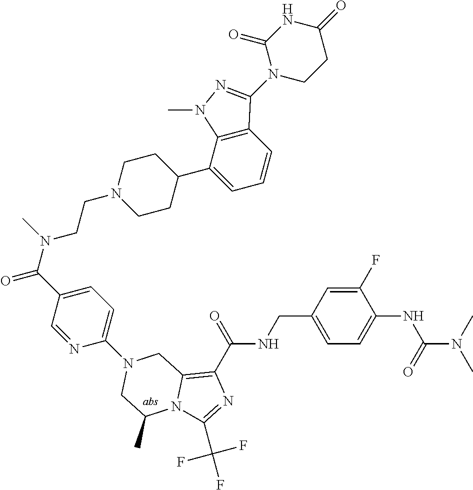 [SMILES: C[C@H]1CN(c2ccc(C(=O)N(C)CCN3CCC(c4cccc5c(N6CCC(=O)NC6=O)nn(C)c45)CC3)cn2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(5-((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE115 | 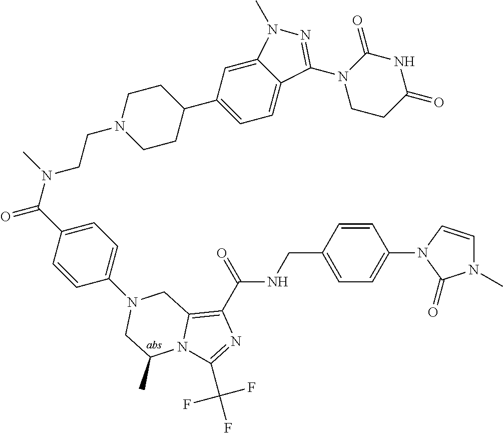 [SMILES: C[C@H]1CN(c2ccc(C(=O)N(C)CCN3CCC(c4ccc5c(N6CCC(=O)NC6=O)nn(C)c5c4)CC3)cc2)Cc2c(C(=O)NCc3ccc(-n4ccn(C)c4=O)cc3)nc(C(F)(F)F)n21] | (S)-7-(4-((2-(4-(3-(2,4- dioxotetrahydropyrimidin-1(2H)- yl)-1-methyl-1H-indazol-6- yl)piperidin-1- yl)ethyl)(methyl)carbamoyl)phenyl)- 5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE116 | 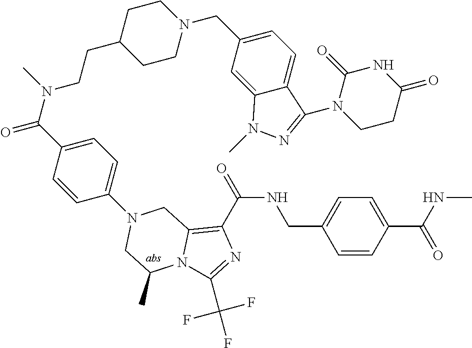 [SMILES: CNC(=O)c1ccc(CNC(=O)c2nc(C(F)(F)F)n3c2CN(c2ccc(C(=O)N(C)CCC4CCN(Cc5ccc6c(N7CCC(=O)NC7=O)nn(C)c6c5)CC4)cc2)C[C@@H]3C)cc1] | (S)-7-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 5-methyl-N-(4- (methylcarbamoyl)benzyl)-3- (trifluoromethyl)-5,6,7,8- tetrahydroimidazo[1,5- a]pyrazine-1-carboxamide |
| VE117 | 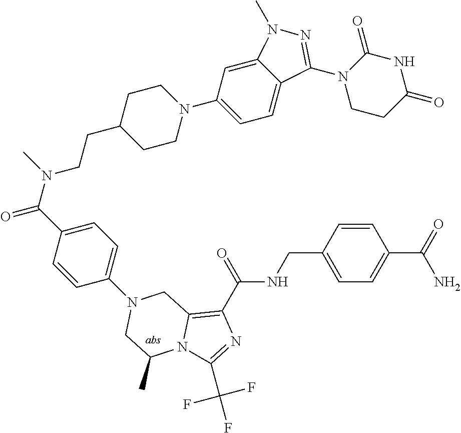 [SMILES: C[C@H]1CN(c2ccc(C(=O)N(C)CCC3CCN(c4ccc5c(N6CCC(=O)NC6=O)nn(C)c5c4)CC3)cc2)Cc2c(C(=O)NCc3ccc(C(N)=O)cc3)nc(C(F)(F)F)n21] | (S)-N-(4-carbamoylbenzyl)-7-(4- ((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE118 | 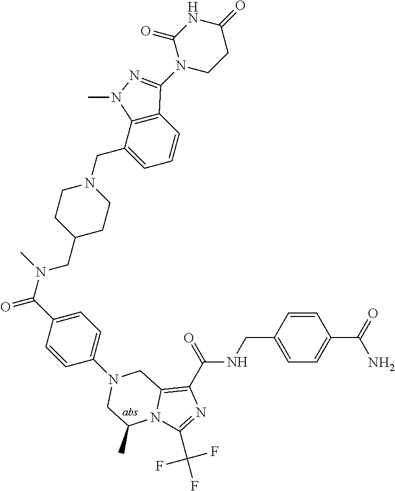 [SMILES: C[C@H]1CN(c2ccc(C(=O)N(C)CC3CCN(Cc4cccc5c(N6CCC(=O)NC6=O)nn(C)c45)CC3)cc2)Cc2c(C(=O)NCc3ccc(C(N)=O)cc3)nc(C(F)(F)F)n21] | (S)-N-(4-carbamoylbenzyl)-7-(4- (((1-((3-(2,4- dioxotetrahydropyrimidin-1(2H)- yl)-1-methyl-1H-indazol-7- yl)methyl)piperidin-4- yl)methyl)(methyl)carbamoyl) phenyl)-5-methyl-3-(trifluoromethyl)- 5,6,7,8-tetrahydroimidazo[1,5- a]pyrazine-1-carboxamide |
| VE119 | 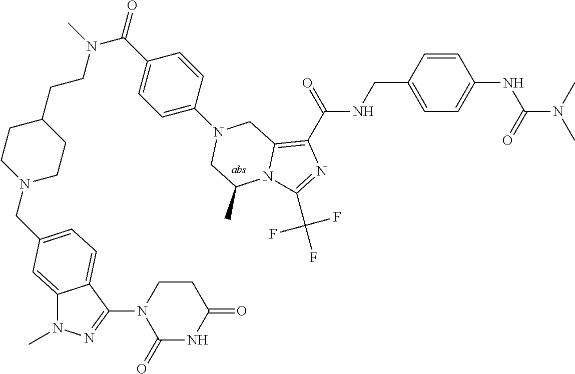 [SMILES: C[C@H]1CN(c2ccc(C(=O)N(C)CCC3CCN(Cc4ccc5c(N6CCC(=O)NC6=O)nn(C)c5c4)CC3)cc2)Cc2c(C(=O)NCc3ccc(NC(=O)N(C)C)cc3)nc(C(F)(F)F)n21] | (S)-N-(4-(3,3- dimethylureido)benzyl)-7-(4-((2- (1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE120 | 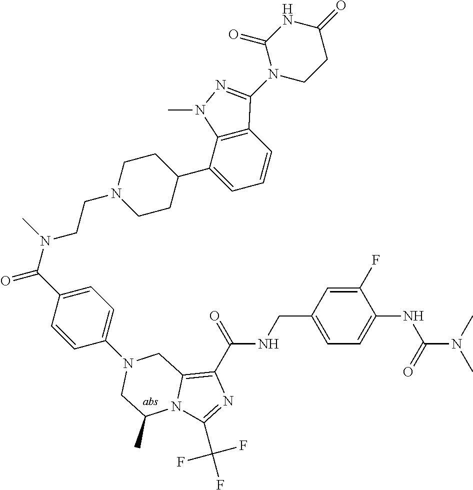 [SMILES: C[C@H]1CN(c2ccc(C(=O)N(C)CCN3CCC(c4cccc5c(N6CCC(=O)NC6=O)nn(C)c45)CC3)cc2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE121 | 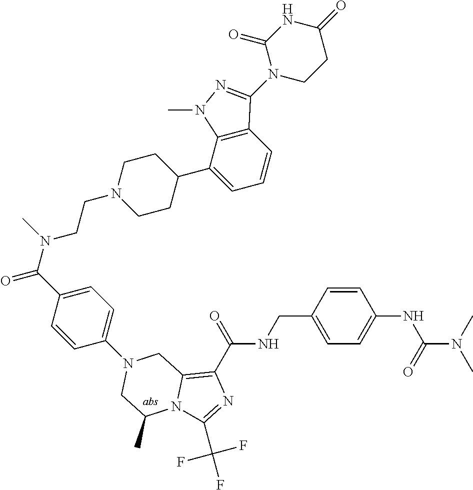 [SMILES: C[C@H]1CN(c2ccc(C(=O)N(C)CCN3CCC(c4cccc5c(N6CCC(=O)NC6=O)nn(C)c45)CC3)cc2)Cc2c(C(=O)NCc3ccc(NC(=O)N(C)C)cc3)nc(C(F)(F)F)n21] | (S)-N-(4-(3,3- dimethylureido)benzyl)-7-(4-((2- (4-(3-(2,4- dioxotetrahydropyrimidin-1(2H)- yl)-1-methyl-1H-indazol-7- yl)piperidin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE122 | 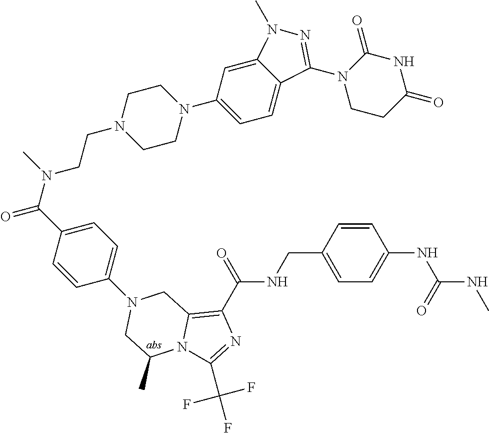 [SMILES: CNC(=O)Nc1ccc(CNC(=O)c2nc(C(F)(F)F)n3c2CN(c2ccc(C(=O)N(C)CCN4CCN(c5ccc6c(N7CCC(=O)NC7=O)nn(C)c6c5)CC4)cc2)C[C@@H]3C)cc1] | (S)-7-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 5-methyl-N-(4-(3- methylureido)benzyl)-3- (trifluoromethyl)-5,6,7,8- tetrahydroimidazo[1,5- a]pyrazine-1-carboxamide |
| VE123 | 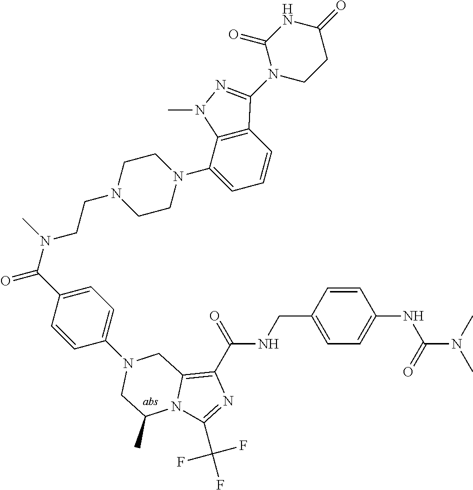 [SMILES: C[C@H]1CN(c2ccc(C(=O)N(C)CCN3CCN(c4cccc5c(N6CCC(=O)NC6=O)nn(C)c45)CC3)cc2)Cc2c(C(=O)NCc3ccc(NC(=O)N(C)C)cc3)nc(C(F)(F)F)n21] | (S)-N-(4-(3,3- dimethylureido)benzyl)-7-(4-((2- (4-(3-(2,4- dioxotetrahydropyrimidin-1(2H)- yl)-1-methyl-1H-indazol-7- yl)piperazin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE124 | 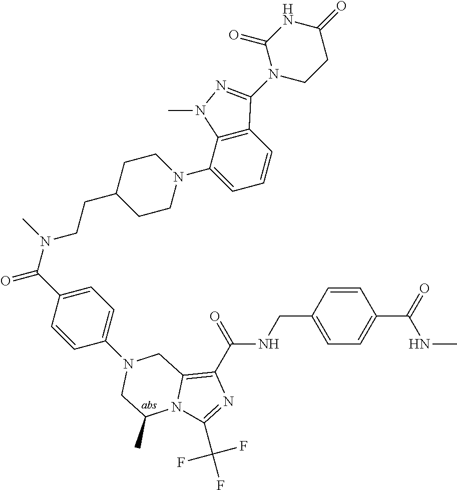 [SMILES: CNC(=O)c1ccc(CNC(=O)c2nc(C(F)(F)F)n3c2CN(c2ccc(C(=O)N(C)CCC4CCN(c5cccc6c(N7CCC(=O)NC7=O)nn(C)c56)CC4)cc2)C[C@@H]3C)cc1] | (S)-7-(4-((2-(1-(3-(2,4- dioxotetrahydropyrimidin-1(2H)- yl)-1-methyl-1H-indazol-7- yl)piperidin-4- yl)ethyl)(methyl)carbamoyl) phenyl)-5-methyl-N-(4- (methylcarbamoyl)benzyl)-3- (trifluoromethyl)-5,6,7,8- tetrahydroimidazo[1,5- a]pyrazine-1-carboxamide |
| VE125 | 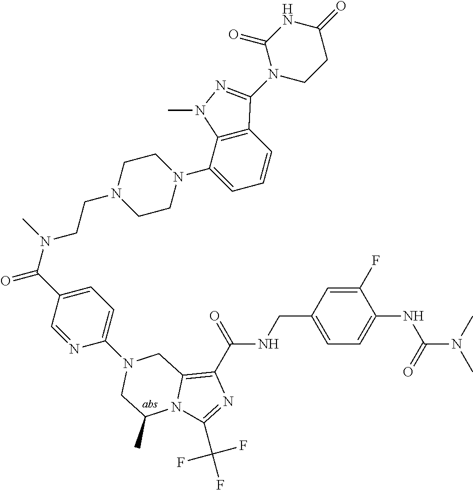 [SMILES: C[C@H]1CN(c2ccc(C(=O)N(C)CCN3CCN(c4cccc5c(N6CCC(=O)NC6=O)nn(C)c45)CC3)cn2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(5-((2-(4-(3-(2,4- dioxotetrahydropyrimidin-1(2H)- yl)-1-methyl-1H-indazol-7- yl)piperazin-1- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE126 | 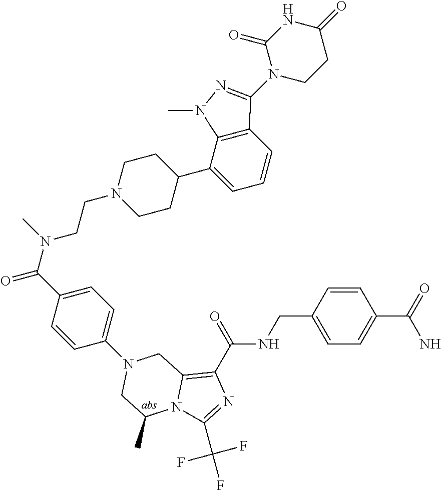 [SMILES: C[C@H]1CN(c2ccc(C(=O)N(C)CCN3CCC(c4cccc5c(N6CCC(=O)NC6=O)nn(C)c45)CC3)cc2)Cc2c(C(=O)NCc3ccc(C(N)=O)cc3)nc(C(F)(F)F)n21] | (S)-N-(4-carbamoylbenzyl)-7-(4- ((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE128 | 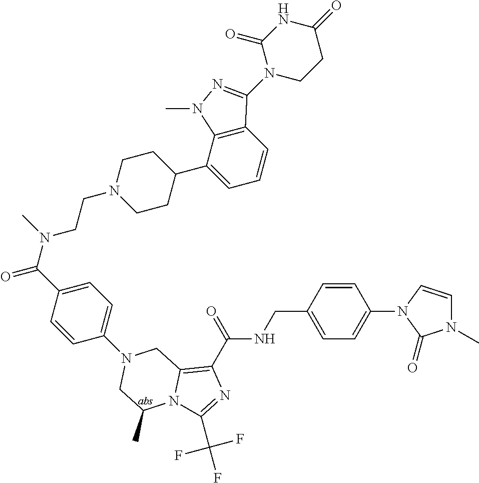 [SMILES: C[C@H]1CN(c2ccc(C(=O)N(C)CCN3CCC(c4cccc5c(N6CCC(=O)NC6=O)nn(C)c45)CC3)cc2)Cc2c(C(=O)NCc3ccc(-n4ccn(C)c4=O)cc3)nc(C(F)(F)F)n21] | (S)-7-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE129 | 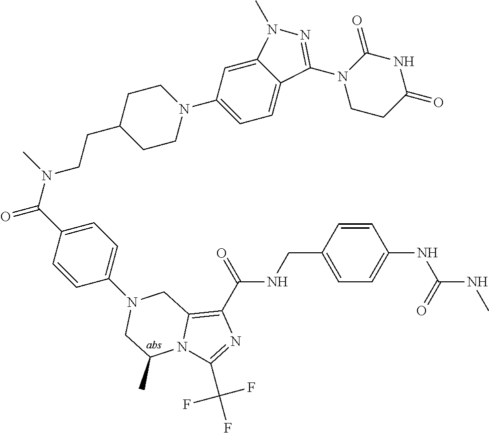 [SMILES: CNC(=O)Nc1ccc(CNC(=O)c2nc(C(F)(F)F)n3c2CN(c2ccc(C(=O)N(C)CCC4CCN(c5ccc6c(N7CCC(=O)NC7=O)nn(C)c6c5)CC4)cc2)C[C@@H]3C)cc1] | (S)-7-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 5-methyl-N-(4-(3- methylureido)benzyl)-3- (trifluoromethyl)-5,6,7,8- tetrahydroimidazo[1,5- a]pyrazine-1-carboxamide |
| VE130 | 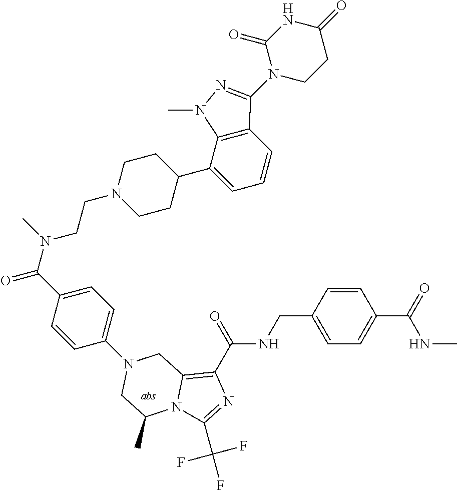 [SMILES: CNC(=O)c1ccc(CNC(=O)c2nc(C(F)(F)F)n3c2CN(c2ccc(C(=O)N(C)CCN4CCC(c5cccc6c(N7CCC(=O)NC7=O)nn(C)c56)CC4)cc2)C[C@@H]3C)cc1] | (S)-7-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 5-methyl-N-(4- (methylcarbamoyl)benzyl)-3- (trifluoromethyl)-5,6,7,8- tetrahydroimidazo[1,5- a]pyrazine-1-carboxamide |
| VE131 | 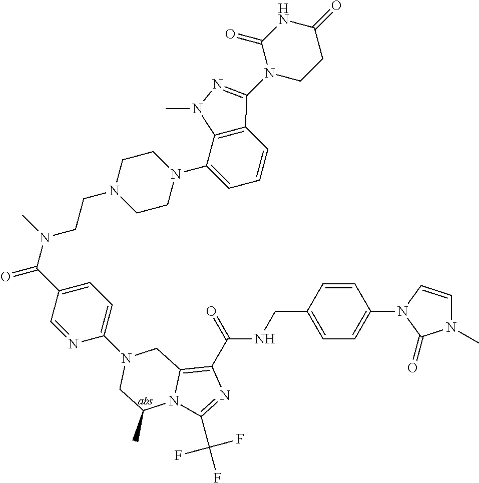 [SMILES: C[C@H]1CN(c2ccc(C(=O)N(C)CCN3CCN(c4cccc5c(N6CCC(=O)NC6=O)nn(C)c45)CC3)cn2)Cc2c(C(=O)NCc3ccc(-n4ccn(C)c4=O)cc3)nc(C(F)(F)F)n21] | (S)-7-(5-((2-(4-(3-(2,4- dioxotetrahydropyrimidin-1(2H)- yl)-1-methyl-1H-indazol-7- yl)piperazin-1- yl)ethyl)(methyl)carbamoyl)pyridin- 2-yl)-5-methyl-N-(4-(3-methyl- 2-oxo-2,3-dihydro-1H-imidazol- 1-yl)benzyl)-3-(trifluoromethyl)- 5,6,7,8-tetrahydroimidazo[1,5- a]pyrazine-1-carboxamide |
| VE132 | 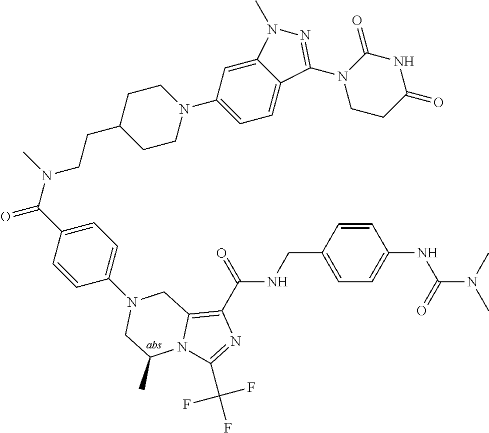 [SMILES: C[C@H]1CN(c2ccc(C(=O)N(C)CCC3CCN(c4ccc5c(N6CCC(=O)NC6=O)nn(C)c5c4)CC3)cc2)Cc2c(C(=O)NCc3ccc(NC(=O)N(C)C)cc3)nc(C(F)(F)F)n21] | (S)-N-(4-(3,3- dimethylureido)benzyl)-7-(4-((2- (1-(3-(2,4- dioxotetrahydropyrimidin-1(2H)- yl)-1-methyl-1H-indazol-6- yl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE133 | 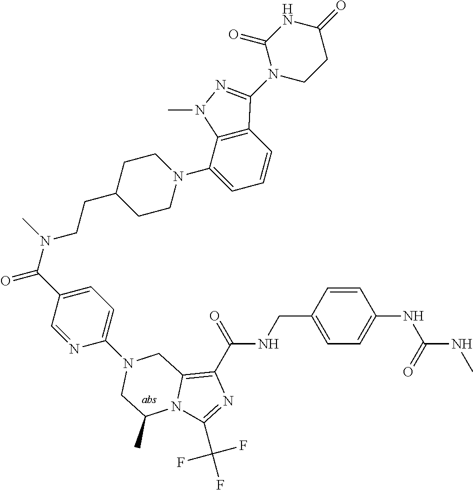 [SMILES: CNC(=O)Nc1ccc(CNC(=O)c2nc(C(F)(F)F)n3c2CN(c2ccc(C(=O)N(C)CCC4CCN(c5cccc6c(N7CCC(=O)NC7=O)nn(C)c56)CC4)cn2)C[C@@H]3C)cc1] | (S)-7-(5-((2-(1-(3-(2,4- dioxotetrahydropyrimidin-1(2H)- yl)-1-methyl-1H-indazol-7- yl)piperidin-4- yl)ethyl)(methyl)carbamoyl)pyridin- 2-yl)-5-methyl-N-(4-(3- methylureido)benzyl)-3- (trifluoromethyl)-5,6,7,8- tetrahydroimidazo[1,5- a]pyrazine-1-carboxamide |
| VE134 | 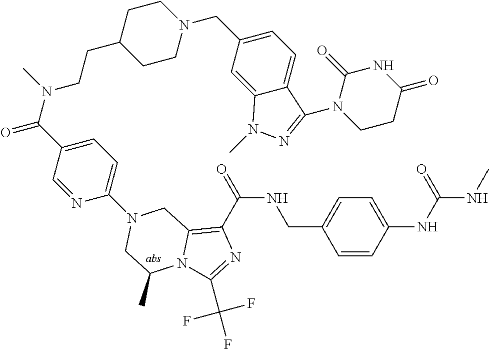 [SMILES: CNC(=O)Nc1ccc(CNC(=O)c2nc(C(F)(F)F)n3c2CN(c2ccc(C(=O)N(C)CCC4CCN(Cc5ccc6c(N7CCC(=O)NC7=O)nn(C)c6c5)CC4)cn2)C[C@@H]3C)cc1] | (S)-7-(5-((2-(1-((3-(2,4- dioxotetrahydropyrimidin-1(2H)- yl)-1-methyl-1H-indazol-6- yl)methyl)piperidin-4- yl)ethyl)(methyl)carbamoyl)pyridin- 2-yl)-5-methyl-N-(4-(3- methylureido)benzyl)-3- (trifluoromethyl)-5,6,7,8- tetrahydroimidazo[1,5- a]pyrazine-1-carboxamide |
| VE135 | 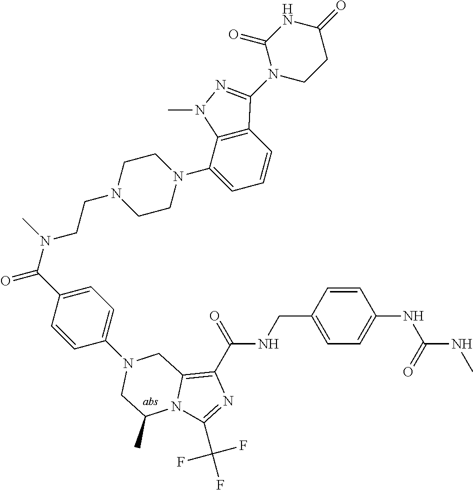 [SMILES: CNC(=O)Nc1ccc(CNC(=O)c2nc(C(F)(F)F)n3c2CN(c2ccc(C(=O)N(C)CCN4CCN(c5cccc6c(N7CCC(=O)NC7=O)nn(C)c56)CC4)cc2)C[C@@H]3C)cc1] | (S)-7-(4-((2-(4-(3-(2,4- dioxotetrahydropyrimidin-1(2H)- yl)-1-methyl-1H-indazol-7- yl) piperazin-1- yl)ethyl)(methyl)carbamoyl)phenyl)- 5-methyl-N-(4-(3- methylureido)benzyl)-3- (trifluoromethyl)-5,6,7,8- tetrahydroimidazo[1,5- a]pyrazine-1-carboxamide |
| VE136 | 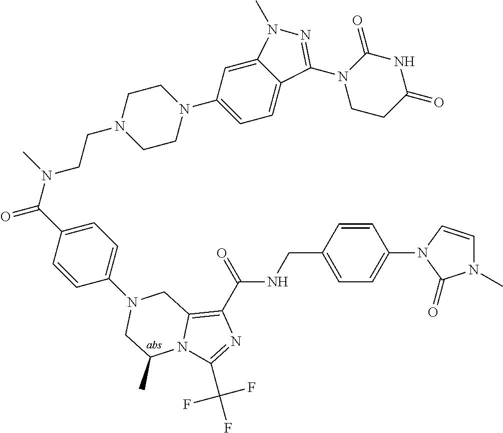 [SMILES: C[C@H]1CN(c2ccc(C(=O)N(C)CCN3CCN(c4ccc5c(N6CCC(=O)NC6=O)nn(C)c5c4)CC3)cc2)Cc2c(C(=O)NCc3ccc(-n4ccn(C)c4=O)cc3)nc(C(F)(F)F)n21] | (S)-7-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE137 | 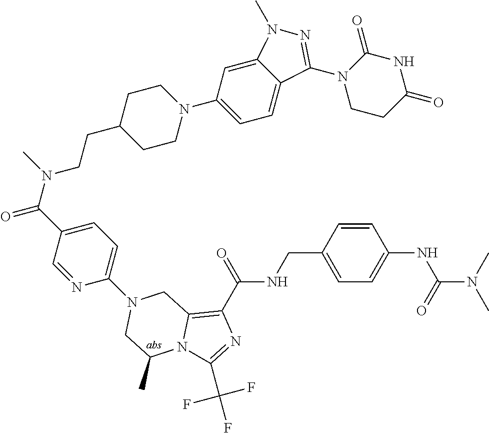 [SMILES: C[C@H]1CN(c2ccc(C(=O)N(C)CCC3CCN(c4ccc5c(N6CCC(=O)NC6=O)nn(C)c5c4)CC3)cn2)Cc2c(C(=O)NCc3ccc(NC(=O)N(C)C)cc3)nc(C(F)(F)F)n21] | (S)-N-(4-(3,3- dimethylureido)benzyl)-7-(5-((2- (1-(3-(2,4- dioxotetrahydropyrimidin-1(2H)- yl)-1-methyl-1H-indazol-6- yl)piperidin-4- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE138 | 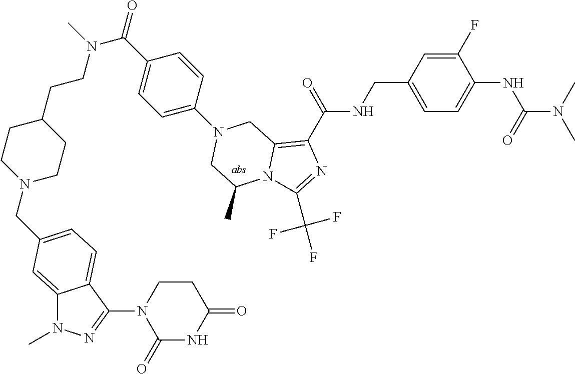 [SMILES: C[C@H]1CN(c2ccc(C(=O)N(C)CCC3CCN(Cc4ccc5c(N6CCC(=O)NC6=O)nn(C)c5c4)CC3)cc2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE139 | 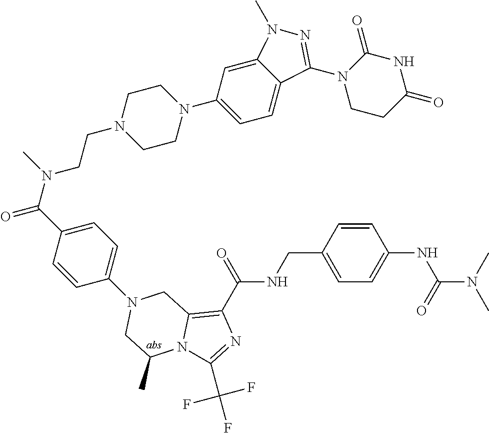 [SMILES: C[C@H]1CN(c2ccc(C(=O)N(C)CCN3CCN(c4ccc5c(N6CCC(=O)NC6=O)nn(C)c5c4)CC3)cc2)Cc2c(C(=O)NCc3ccc(NC(=O)N(C)C)cc3)nc(C(F)(F)F)n21] | (S)-N-(4-(3,3- dimethylureido)benzyl)-7-(4-((2- (4-(3-(2,4- dioxotetrahydropyrimidin-1(2H)- yl)-1-methyl-1H-indazol-6- yl)piperazin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE140 | 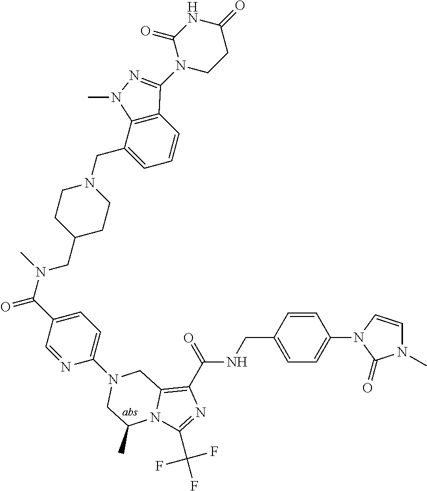 [SMILES: C[C@H]1CN(c2ccc(C(=O)N(C)CC3CCN(Cc4cccc5c(N6CCC(=O)NC6=O)nn(C)c45)CC3)cn2)Cc2c(C(=O)NCc3ccc(-n4ccn(C)c4=O)cc3)nc(C(F)(F)F)n21] | (S)-7-(5-(((1-((3-(2,4- dioxotetrahydropyrimidin-1(2H)- yl)-1-methyl-1H-indazol-7- yl)methyl)piperidin-4- yl)methyl)(methyl)carbamoyl) pyridin-2-yl)-5-methyl-N-(4-(3- methyl-2-oxo-2,3-dihydro-1H- imidazol-1-yl)benzyl)-3- (trifluoromethyl)-5,6,7,8- tetrahydroimidazo[1,5- a]pyrazine-1-carboxamide |
| VE141 | 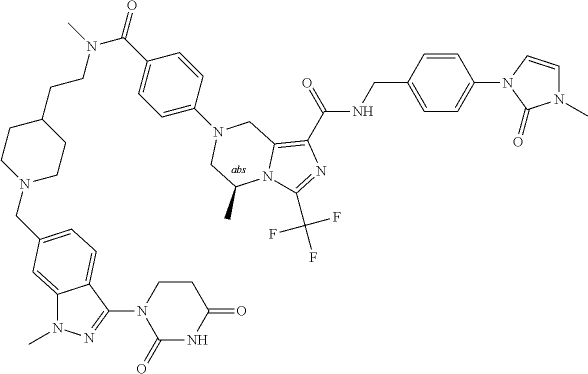 [SMILES: C[C@H]1CN(c2ccc(C(=O)N(C)CCC3CCN(Cc4ccc5c(N6CCC(=O)NC6=O)nn(C)c5c4)CC3)cc2)Cc2c(C(=O)NCc3ccc(-n4ccn(C)c4=O)cc3)nc(C(F)(F)F)n21] | (S)-7-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE142 | 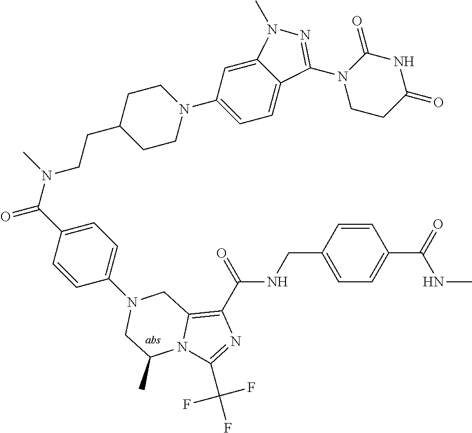 [SMILES: CNC(=O)c1ccc(CNC(=O)c2nc(C(F)(F)F)n3c2CN(c2ccc(C(=O)N(C)CCC4CCN(c5ccc6c(N7CCC(=O)NC7=O)nn(C)c6c5)CC4)cc2)C[C@@H]3C)cc1] | (S)-7-(4-((2-(1-(3-(2,4- dioxotetrahydropyrimidin-1(2H)- yl)-1-methyl-1H-indazol-6- yl)piperidin-4- yl)ethyl)(methyl)carbamoyl) phenyl)-5-methyl-N-(4- (methylcarbamoyl)benzyl)-3- (trifluoromethyl)-5,6,7,8- tetrahydroimidazo[1,5- a]pyrazine-1-carboxamide |
| VE143 | 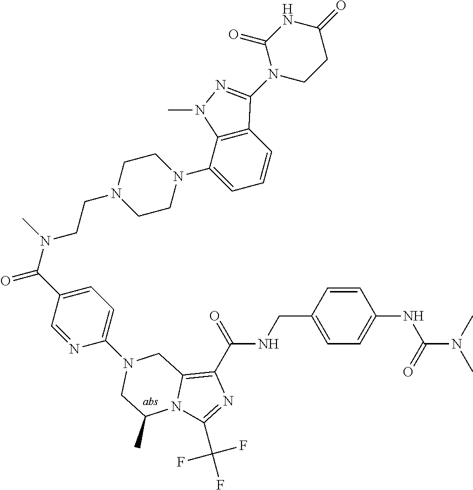 [SMILES: C[C@H]1CN(c2ccc(C(=O)N(C)CCN3CCN(c4cccc5c(N6CCC(=O)NC6=O)nn(C)c45)CC3)cn2)Cc2c(C(=O)NCc3ccc(NC(=O)N(C)C)cc3)nc(C(F)(F)F)n21] | (S)-N-(4-(3,3- dimethylureido)benzyl)-7-(5-((2- (1-(3-(2,4- dioxotetrahydropyrimidin-1(2H)- yl)-1-methyl-1H-indazol-7- yl)piperidin-4- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE144 | 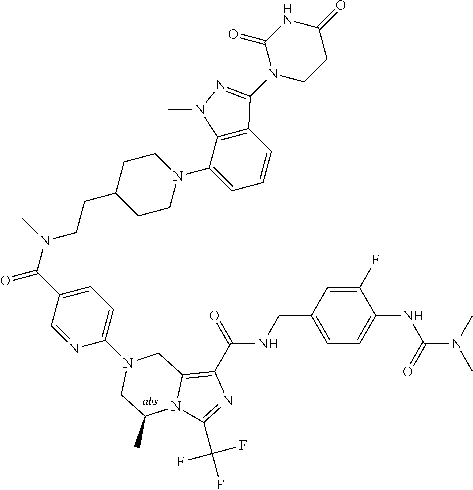 [SMILES: C[C@H]1CN(c2ccc(C(=O)N(C)CCC3CCN(c4cccc5c(N6CCC(=O)NC6=O)nn(C)c45)CC3)cn2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(5-((2-(1-(3-(2,4- dioxotetrahydropyrimidin-1(2H)- yl)-1-methyl-1H-indazol-7- yl)piperidin-4- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE145 | 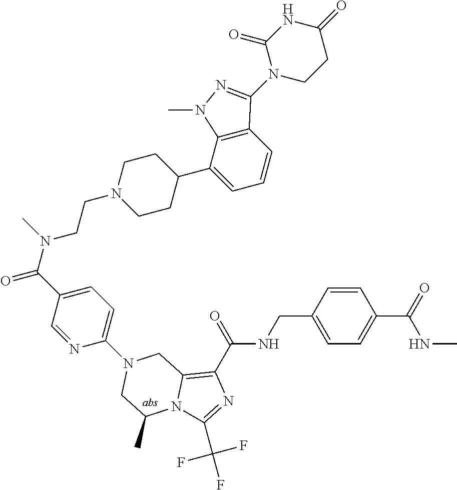 [SMILES: CNC(=O)c1ccc(CNC(=O)c2nc(C(F)(F)F)n3c2CN(c2ccc(C(=O)N(C)CCN4CCC(c5cccc6c(N7CCC(=O)NC7=O)nn(C)c56)CC4)cn2)C[C@@H]3C)cc1] | (S)-7-(5-((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-5-methyl-N-(4- (methylcarbamoyl)benzyl)-3- (trifluoromethyl)-5,6,7,8- tetrahydroimidazo[1,5- a]pyrazine-1-carboxamide |
| VE146 | 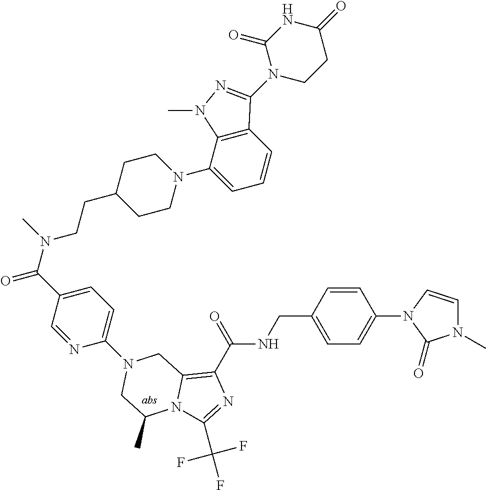 [SMILES: C[C@H]1CN(c2ccc(C(=O)N(C)CCC3CCN(c4cccc5c(N6CCC(=O)NC6=O)nn(C)c45)CC3)cn2)Cc2c(C(=O)NCc3ccc(-n4ccn(C)c4=O)cc3)nc(C(F)(F)F)n21] | (S)-7-(5-((2-(1-(3-(2,4- dioxotetrahydropyrimidin-1(2H)- yl)-1-methyl-1H-indazol-7- yl)piperidin-4- yl)ethyl)(methyl)carbamoyl)pyridin- 2-yl)-5-methyl-N-(4-(3-methyl- 2-oxo-2,3-dihydro-1H-imidazol- 1-yl)benzyl)-3-(trifluoromethyl)- 5,6,7,8-tetrahydroimidazo[1,5- a]pyrazine-1-carboxamide |
| VE147 | 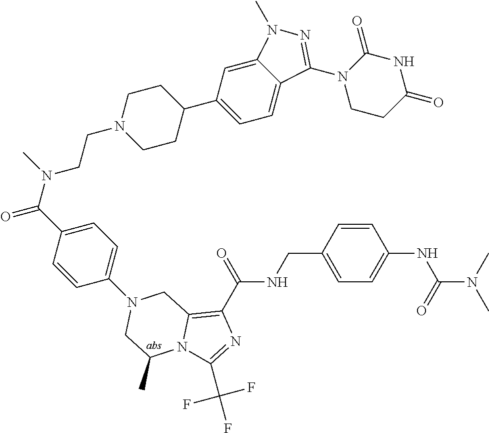 [SMILES: C[C@H]1CN(c2ccc(C(=O)N(C)CCN3CCC(c4ccc5c(N6CCC(=O)NC6=O)nn(C)c5c4)CC3)cc2)Cc2c(C(=O)NCc3ccc(NC(=O)N(C)C)cc3)nc(C(F)(F)F)n21] | (S)-N-(4-(3,3-dimethylureido)-3- fluorobenzyl)-7-(4-((2-(4-(3-(2,4- dioxotetrahydropyrimidin-1(2H)- yl)-1-methyl-1H-indazol-6- yl)piperidin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE148 | 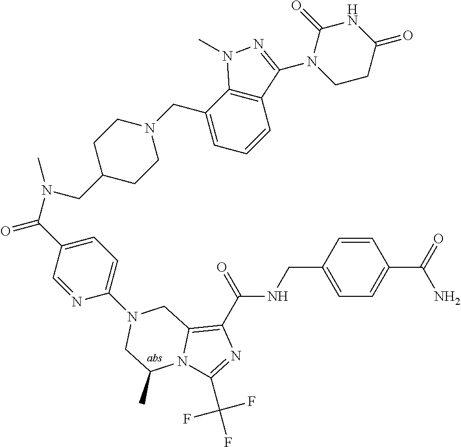 [SMILES: C[C@H]1CN(c2ccc(C(=O)N(C)CC3CCN(Cc4cccc5c(N6CCC(=O)NC6=O)nn(C)c45)CC3)cn2)Cc2c(C(=O)NCc3ccc(C(N)=O)cc3)nc(C(F)(F)F)n21] | (S)-N-(4-carbamoylbenzyl)-7-(5- (((1-((3-(2,4- dioxotetrahydropyrimidin-1(2H)- yl)-1-methyl-1H-indazol-7- yl)methyl)piperidin-4- yl)methyl)(methyl)carbamoyl) pyridin-2-yl)-5-methyl-3- (trifluoromethyl)-5,6,7,8- tetrahydroimidazo[1,5- a]pyrazine-1-carboxamide |
| VE150 | 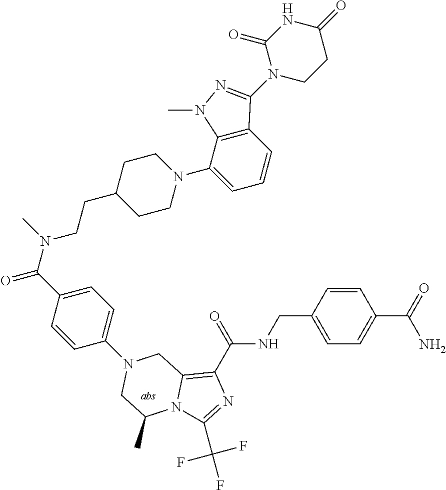 [SMILES: C[C@H]1CN(c2ccc(C(=O)N(C)CCC3CCN(c4cccc5c(N6CCC(=O)NC6=O)nn(C)c45)CC3)cc2)Cc2c(C(=O)NCc3ccc(C(N)=O)cc3)nc(C(F)(F)F)n21] | (S)-N-(4-carbamoylbenzyl)-7-(4- ((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE151 | 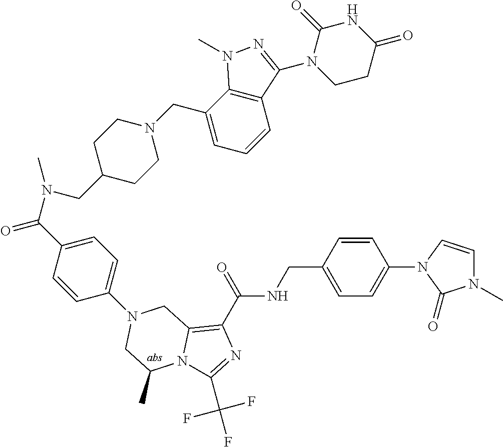 [SMILES: C[C@H]1CN(c2ccc(C(=O)N(C)CC3CCN(Cc4cccc5c(N6CCC(=O)NC6=O)nn(C)c45)CC3)cc2)Cc2c(C(=O)NCc3ccc(-n4ccn(C)c4=O)cc3)nc(C(F)(F)F)n21] | (S)-7-(4-(((1-((3-(2,4- dioxotetrahydropyrimidin-1(2H)- yl)-1-methyl-1H-indazol-7- yl)methyl)piperidin-4- yl)methyl)(methyl)carbamoyl) phenyl)-5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE152 | 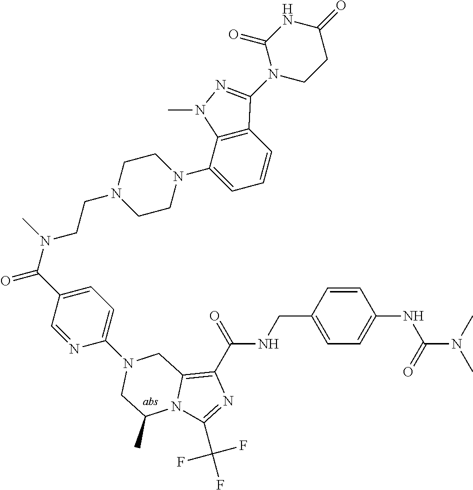 [SMILES: C[C@H]1CN(c2ccc(C(=O)N(C)CCN3CCN(c4cccc5c(N6CCC(=O)NC6=O)nn(C)c45)CC3)cn2)Cc2c(C(=O)NCc3ccc(NC(=O)N(C)C)cc3)nc(C(F)(F)F)n21] | (S)-N-(4-(3,3- dimethylureido)benzyl)-7-(5-((2- (4-(3-(2,4- dioxotetrahydropyrimidin-1(2H)- yl)-1-methyl-1H-indazol-7- yl)piperazin-1- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE153 | 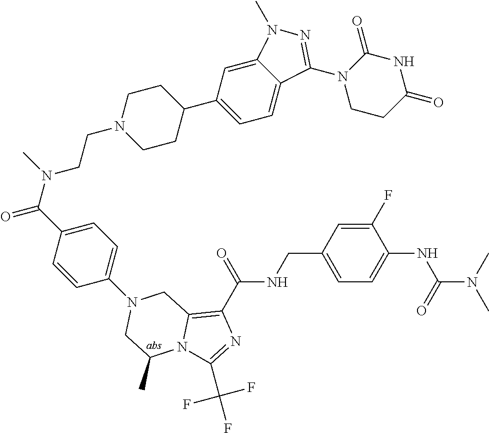 [SMILES: C[C@H]1CN(c2ccc(C(=O)N(C)CCN3CCC(c4ccc5c(N6CCC(=O)NC6=O)nn(C)c5c4)CC3)cc2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE154 | 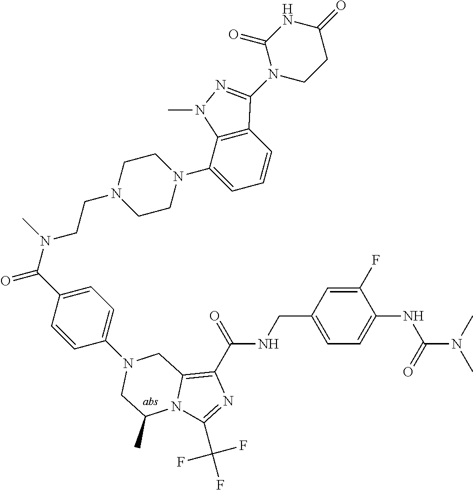 [SMILES: C[C@H]1CN(c2ccc(C(=O)N(C)CCN3CCN(c4cccc5c(N6CCC(=O)NC6=O)nn(C)c45)CC3)cc2)Cc2c(C(=O)NCc3ccc(NC(=O)N(C)C)c(F)c3)nc(C(F)(F)F)n21] | (S)-N-(4-(3,3-dimethylureido)-3- fluorobenzyl)-7-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 5-methyl-3-(trifluoromethyl)- 5,6,7,8-tetrahydroimidazo[1,5- a]pyrazine-1-carboxamide |
| VE156 | 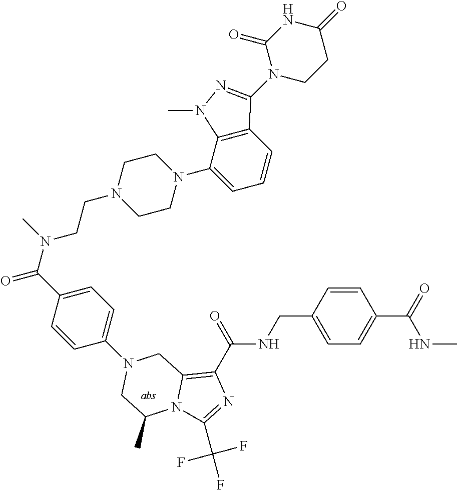 [SMILES: CNC(=O)c1ccc(CNC(=O)c2nc(C(F)(F)F)n3c2CN(c2ccc(C(=O)N(C)CCN4CCN(c5cccc6c(N7CCC(=O)NC7=O)nn(C)c56)CC4)cc2)C[C@@H]3C)cc1] | (S)-7-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 5-methyl-N-(4- (methylcarbamoyl)benzyl)-3- (trifluoromethyl)-5,6,7,8- tetrahydroimidazo[1,5- a]pyrazine-1-carboxamide |
| VE157 | 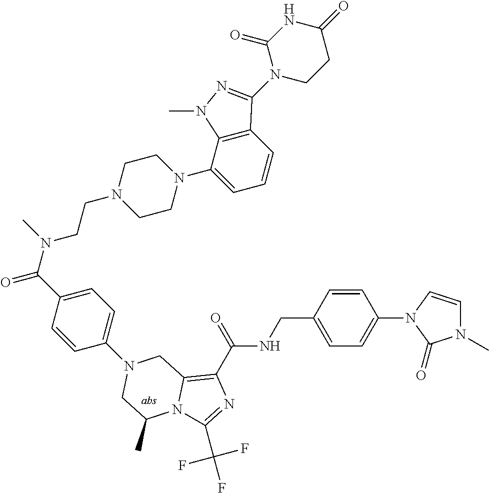 [SMILES: C[C@H]1CN(c2ccc(C(=O)N(C)CCN3CCN(c4cccc5c(N6CCC(=O)NC6=O)nn(C)c45)CC3)cc2)Cc2c(C(=O)NCc3ccc(-n4ccn(C)c4=O)cc3)nc(C(F)(F)F)n21] | (S)-7-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE158 | 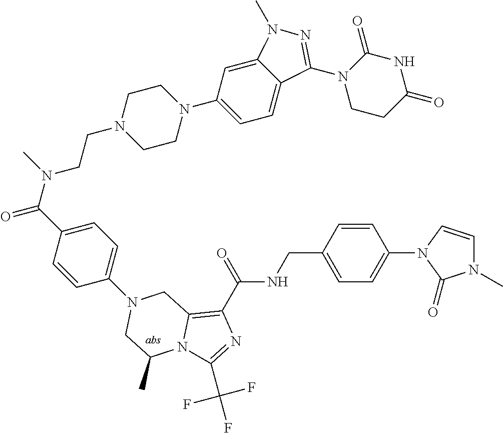 [SMILES: C[C@H]1CN(c2ccc(C(=O)N(C)CCN3CCN(c4ccc5c(N6CCC(=O)NC6=O)nn(C)c5c4)CC3)cc2)Cc2c(C(=O)NCc3ccc(-n4ccn(C)c4=O)cc3)nc(C(F)(F)F)n21] | (S)-7-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE159 | 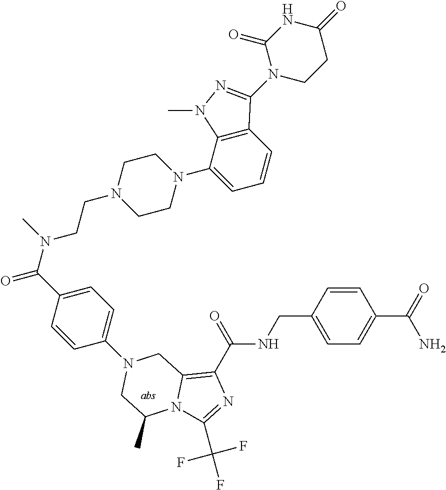 [SMILES: C[C@H]1CN(c2ccc(C(=O)N(C)CCN3CCN(c4cccc5c(N6CCC(=O)NC6=O)nn(C)c45)CC3)cc2)Cc2c(C(=O)NCc3ccc(C(N)=O)cc3)nc(C(F)(F)F)n21] | (S)-N-(4-carbamoylbenzyl)-7-(5- ((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-5-methyl-3- (trifluoromethyl)-5,6,7,8- tetrahydroimidazo[1,5- a]pyrazine-1-carboxamide |
| VE160 | 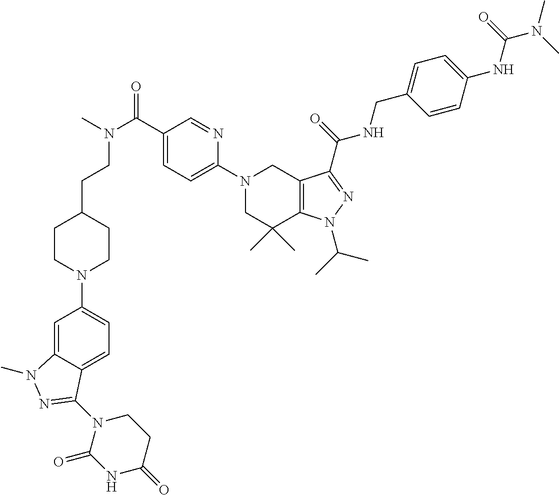 | N-(4-(3,3- dimethylureido)benzyl)-5-(5-((2- (1-(3-(2,4- dioxotetrahydropyrimidin-1(2H)- yl)-1-methyl-1H-indazol-6- yl)piperidin-4- yl)ethyl)(methyl)carbamoyl)pyridin- 2-yl)-1-isopropyl-7,7-dimethyl- 4,5,6,7-tetrahydro-1H- pyrazolo[4,3-c]pyridine-3- carboxamide |
| VE162 | 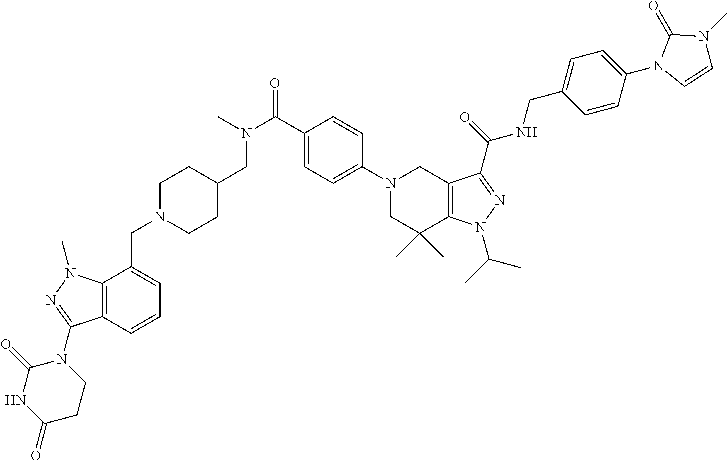 [SMILES: CC(C)n1nc(C(=O)NCc2ccc(-n3ccn(C)c3=O)cc2)c2c1C(C)(C)CN(c1ccc(C(=O)N(C)CC3CCN(Cc4cccc5c(N6CCC(=O)NC6=O)nn(C)c45)CC3)cc1)C2] | 5-(4-(((1-((3-(2,4- dioxotetrahydropyrimidin-1(2H)- yl)-1-methyl-1H-indazol-7- yl)methyl)piperidin-4- yl)methyl)(methyl)carbamoyl) phenyl)-1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE163 | 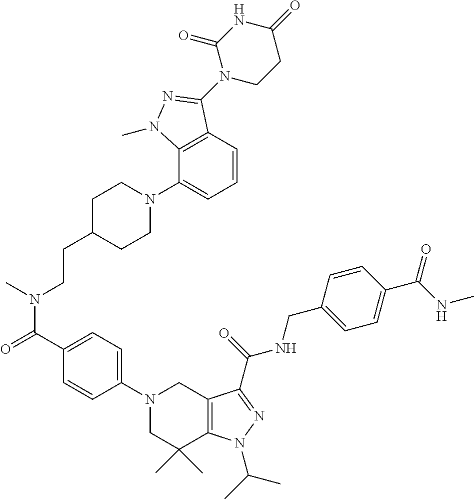 | 5-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7,7-dimethyl-N- (4-(methylcarbamoyl)benzyl)- 4,5,6,7-tetrahydro-1H- pyrazolo[4,3-c]pyridine-3- carboxamide |
| VE164 | 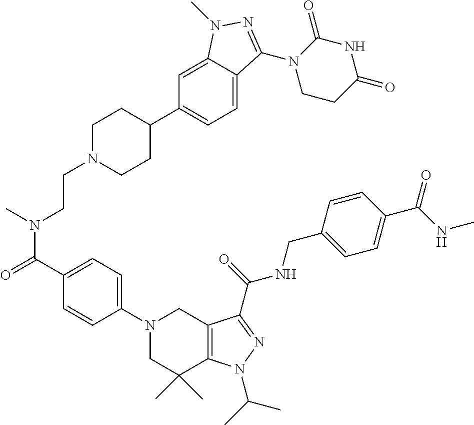 | 5-(4-((2-(4-(3-(2,4- dioxotetrahydropyrimidin-1(2H)- yl)-1-methyl-1H-indazol-6- yl)piperidin-1- yl)ethyl)(methyl)carbamoyl)phenyl)- 1-isopropyl-7,7-dimethyl-N- (4-(methylcarbamoyl)benzyl)- 4,5,6,7-tetrahydro-1H- pyrazolo[4,3-c]pyridine-3- carboxamide |
| VE165 | 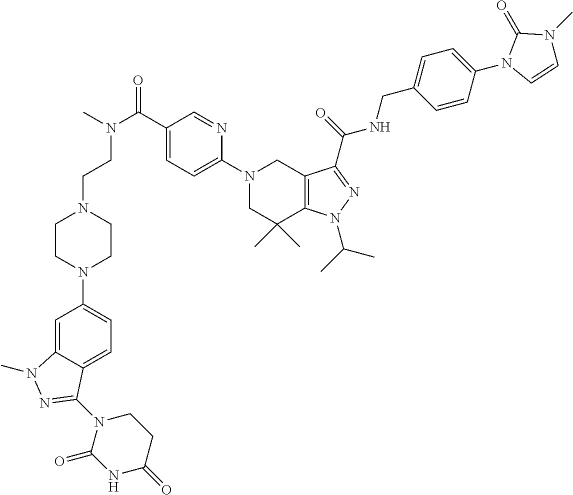 | 5-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE167 | 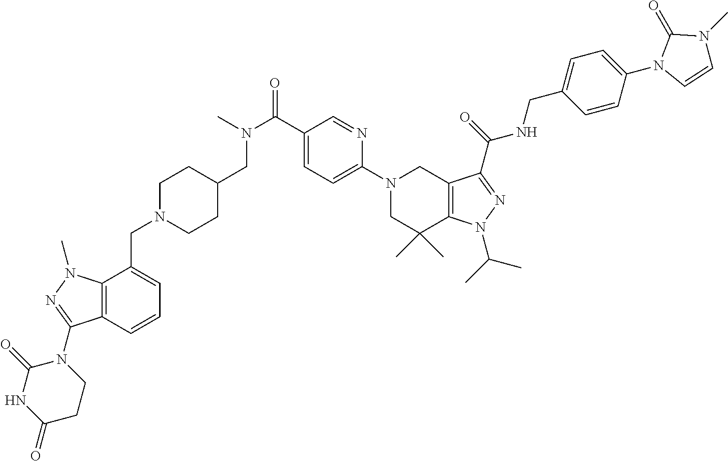 [SMILES: CC(C)n1nc(C(=O)NCc2ccc(-n3ccn(C)c3=O)cc2)c2c1C(C)(C)CN(c1ccc(C(=O)N(C)CC3CCN(Cc4cccc5c(N6CCC(=O)NC6=O)nn(C)c45)CC3)cn1)C2] | 5-(5-(((1-((3-(2,4- dioxotetrahydropyrimidin-1(2H)- yl)-1-methyl-1H-indazol-7- yl)methyl)piperidin-4- yl)methyl)(methyl)carbamoyl) pyridin-2-yl)-1-isopropyl-7,7- dimethyl-N-(4-(3-methyl-2-oxo- 2,3-dihydro-1H-imidazol-1- yl)benzyl)-4,5,6,7-tetrahydro-1H- pyrazolo[4,3-c]pyridine-3- carboxamide |
| VE168 | 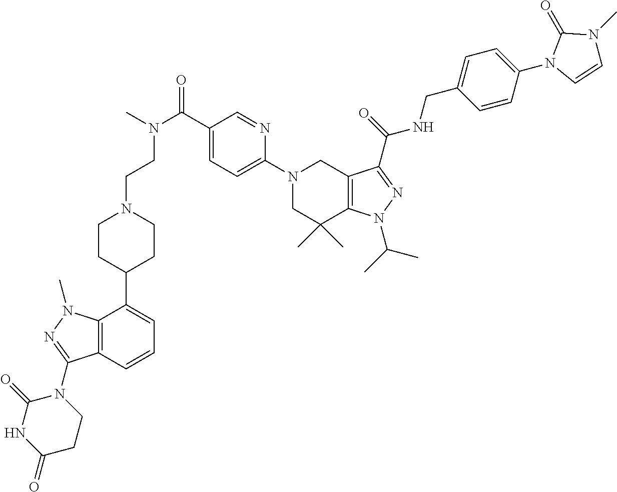 [SMILES: CC(C)n1nc(C(=O)NCc2ccc(-n3ccn(C)c3=O)cc2)c2c1C(C)(C)CN(c1ccc(C(=O)N(C)CCN3CCC(c4cccc5c(N6CCC(=O)NC6=O)nn(C)c45)CC3)cn1)C2] | 5-(5-((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-1-isopropyl-7,7-dimethyl- N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE171 | 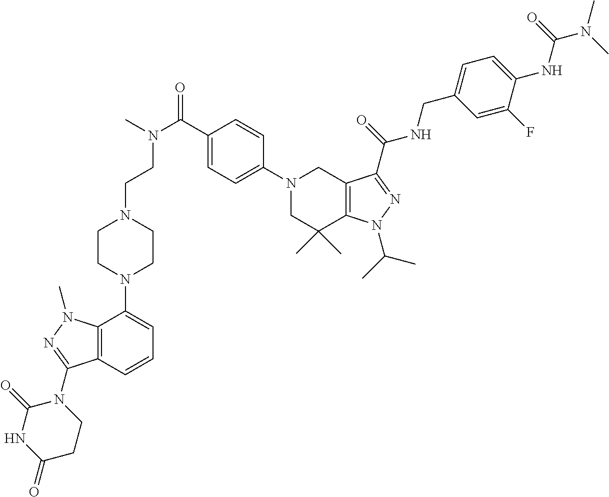 [SMILES: CC(C)n1nc(C(=O)NCc2ccc(NC(=O)N(C)C)c(F)c2)c2c1C(C)(C)CN(c1ccc(C(=O)N(C)CCN3CCN(c4cccc5c(N6CCC(=O)NC6=O)nn(C)c45)CC3)cc1)C2] | N-(4-(3,3-dimethylureido)-3- fluorobenzyl)-5-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE172 | 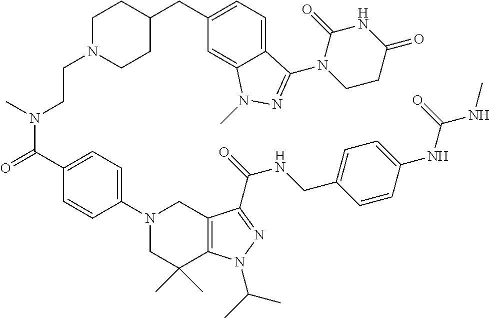 | 5-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 1-isopropyl-7,7-dimethyl-N- (4-(3-methylureido)benzyl)- 4,5,6,7-tetrahydro-1H- pyrazolo[4,3-c]pyridine-3- carboxamide |
| VE173 | 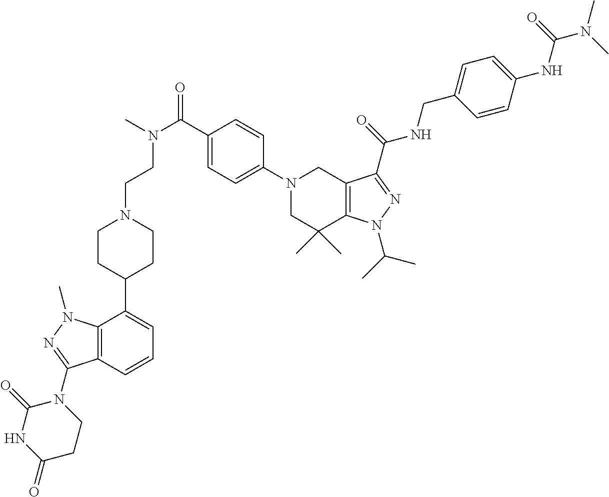 [SMILES: CC(C)n1nc(C(=O)NCc2ccc(NC(=O)N(C)C)cc2)c2c1C(C)(C)CN(c1ccc(C(=O)N(C)CCN3CCC(c4cccc5c(N6CCC(=O)NC6=O)nn(C)c45)CC3)cc1)C2] | N-(4-(3,3- dimethylureido)benzyl)-5-(4-((2- (4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE175 | 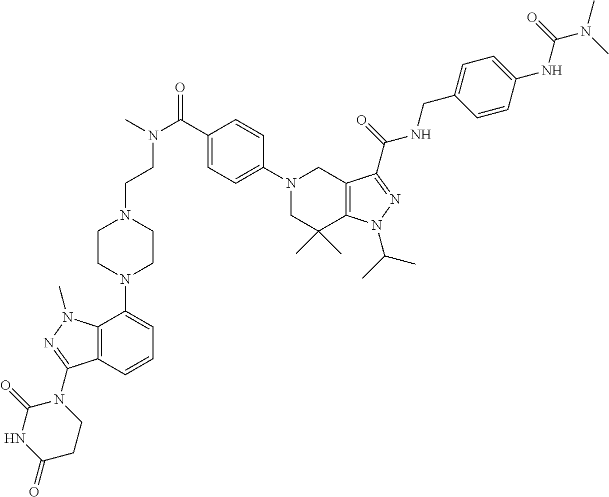 [SMILES: CC(C)n1nc(C(=O)NCc2ccc(NC(=O)N(C)C)cc2)c2c1C(C)(C)CN(c1ccc(C(=O)N(C)CCN3CCN(c4cccc5c(N6CCC(=O)NC6=O)nn(C)c45)CC3)cc1)C2] | N-(4-(3,3- dimethylureido)benzyl)-5-(4-((2- (4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE176 | 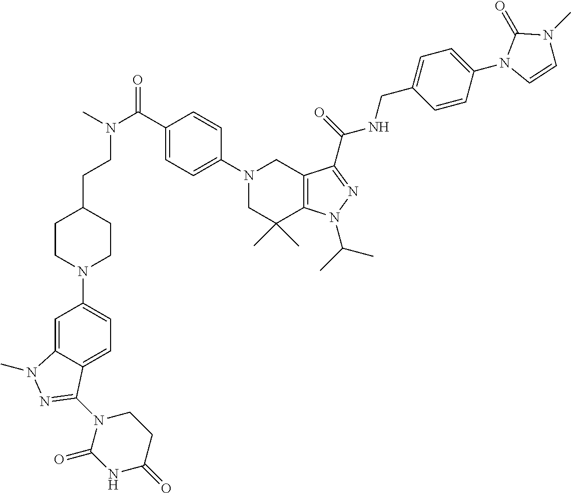 | 5-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE177 | 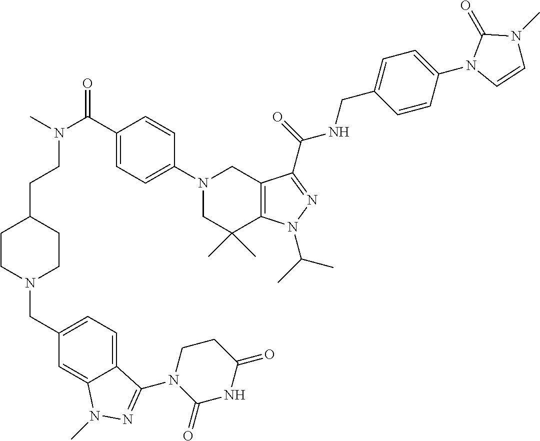 [SMILES: CC(C)n1nc(C(=O)NCc2ccc(-n3ccn(C)c3=O)cc2)c2c1C(C)(C)CN(c1ccc(C(=O)N(C)CCC3CCN(Cc4ccc5c(N6CCC(=O)NC6=O)nn(C)c5c4)CC3)cc1)C2] | 5-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE178 | 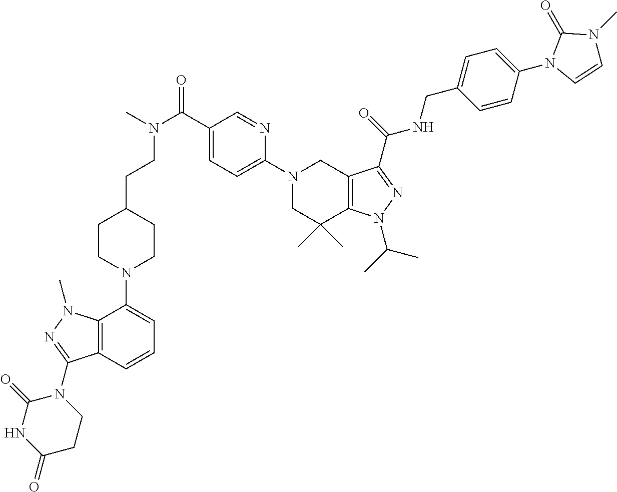 [SMILES: CC(C)n1nc(C(=O)NCc2ccc(-n3ccn(C)c3=O)cc2)c2c1C(C)(C)CN(c1ccc(C(=O)N(C)CCC3CCN(c4cccc5c(N6CCC(=O)NC6=O)nn(C)c45)CC3)cn1)C2] | 5-(5-((2-(1-(3-(2,4- dioxotetrahydropyrimidin-1(2H)- yl)-1-methyl-1H-indazol-7- yl)piperidin-4- yl)ethyl)(methyl)carbamoyl)pyridin- 2-yl)-1-isopropyl-7,7-dimethyl- N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE179 | 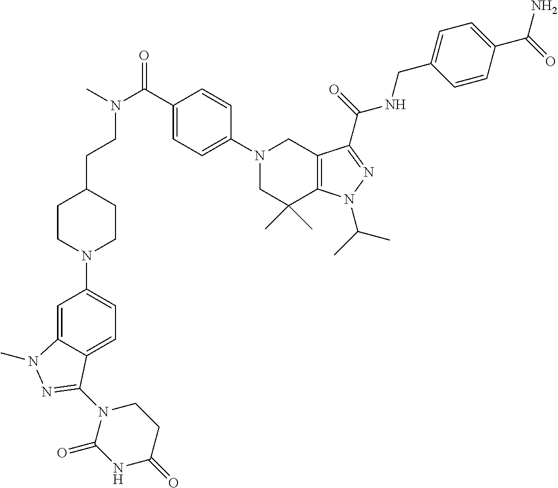 [SMILES: CC(C)n1nc(C(=O)NCc2ccc(C(N)=O)cc2)c2c1C(C)(C)CN(c1ccc(C(=O)N(C)CCC3CCN(c4ccc5c(N6CCC(=O)NC6=O)nn(C)c5c4)CC3)cc1)C2] | N-(4-carbamoylbenzyl)-5-(4-((2- (1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE180 | 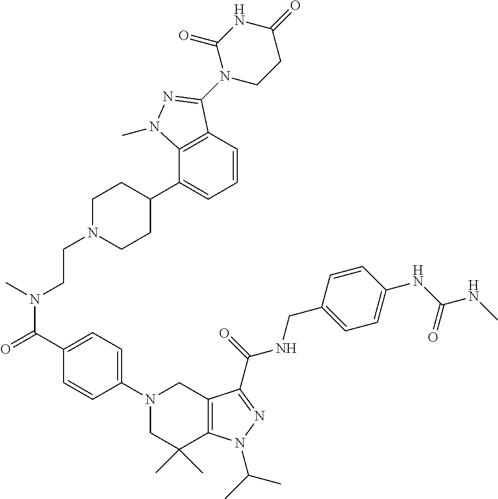 | 5-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7,7-dimethyl-N- (4-(3-methylureido)benzyl)- 4,5,6,7-tetrahydro-1H- pyrazolo[4,3-c]pyridine-3- carboxamide |
| VE181 | 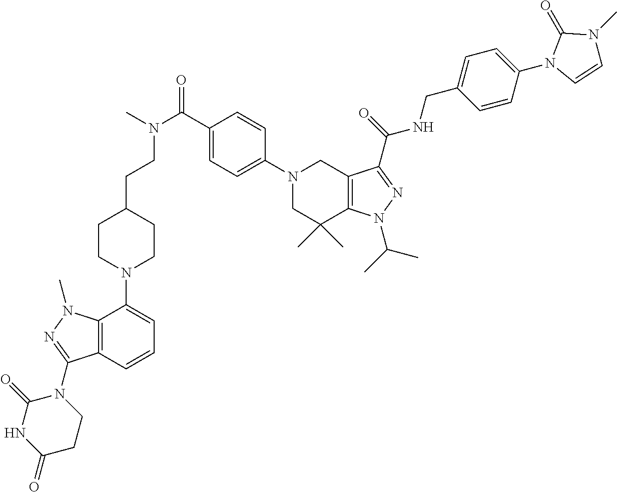 [SMILES: CC(C)n1nc(C(=O)NCc2ccc(-n3ccn(C)c3=O)cc2)c2c1C(C)(C)CN(c1ccc(C(=O)N(C)CCC3CCN(c4cccc5c(N6CCC(=O)NC6=O)nn(C)c45)CC3)cc1)C2] | 5-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE182 | 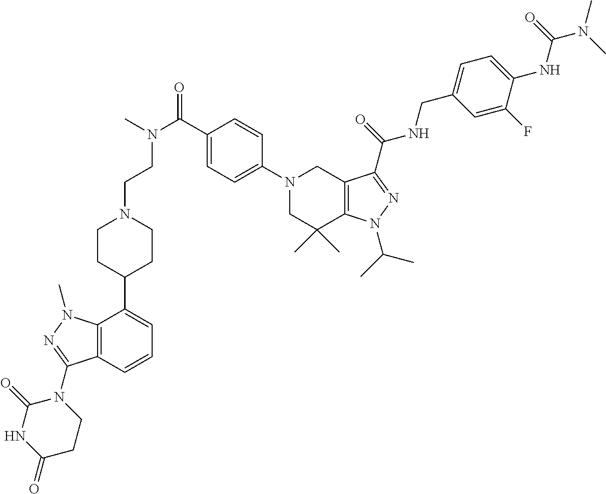 [SMILES: CC(C)n1nc(C(=O)NCc2ccc(NC(=O)N(C)C)c(F)c2)c2c1C(C)(C)CN(c1ccc(C(=O)N(C)CCN3CCC(c4cccc5c(N6CCC(=O)NC6=O)nn(C)c45)CC3)cc1)C2] | N-(4-(3,3-dimethylureido)-3- fluorobenzyl)-5-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE183 | 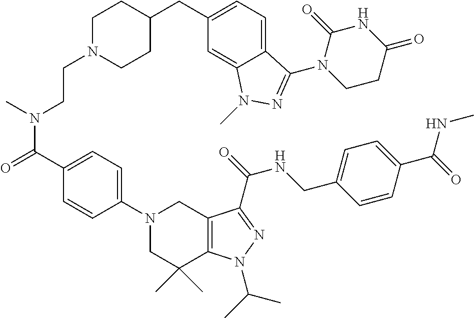 | 5-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 1-isopropyl-7,7-dimethyl-N- (4-(methylcarbamoyl)benzyl)- 4,5,6,7-tetrahydro-1H- pyrazolo[4,3-c]pyridine-3- carboxamide |
| VE184 | 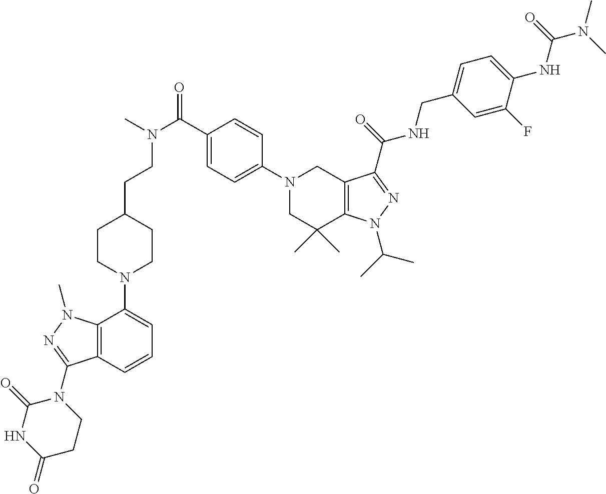 [SMILES: CC(C)n1nc(C(=O)NCc2ccc(NC(=O)N(C)C)c(F)c2)c2c1C(C)(C)CN(c1ccc(C(=O)N(C)CCC3CCN(c4cccc5c(N6CCC(=O)NC6=O)nn(C)c45)CC3)cc1)C2] | N-(4-(3,3-dimethylureido)-3- fluorobenzyl)-5-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE185 | 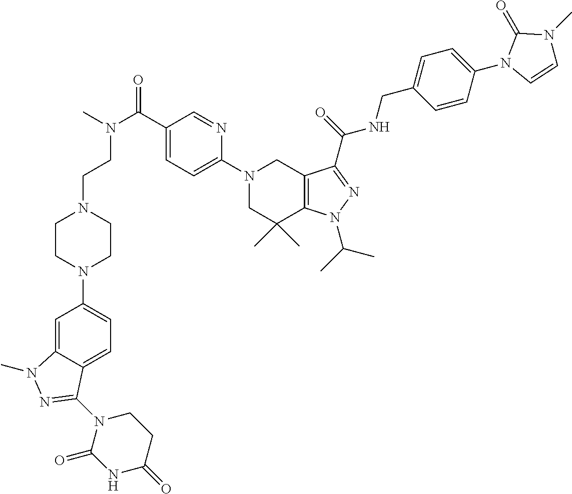 | 5-(5-((2-(4-(3-(2,4- dioxotetrahydropyrimidin-1(2H)- yl)-1-methyl-1H-indazol-6- yl)piperazin-1- yl)ethyl)(methyl)carbamoyl)pyridin- 2-yl)-1-isopropyl-7,7-dimethyl- N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE186 | 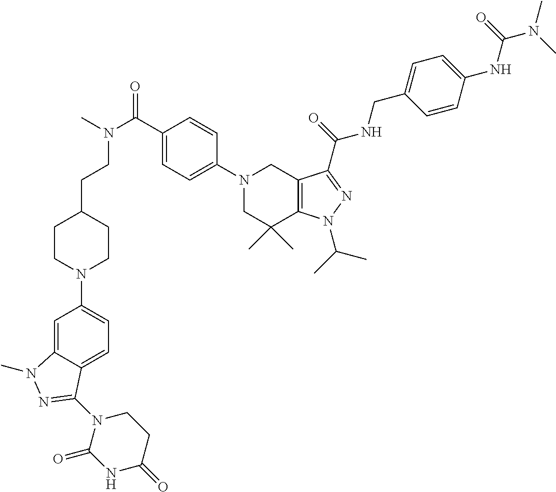 | N-(4-(3,3- dimethylureido)benzyl)-5-(4-((2- (1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE187 | 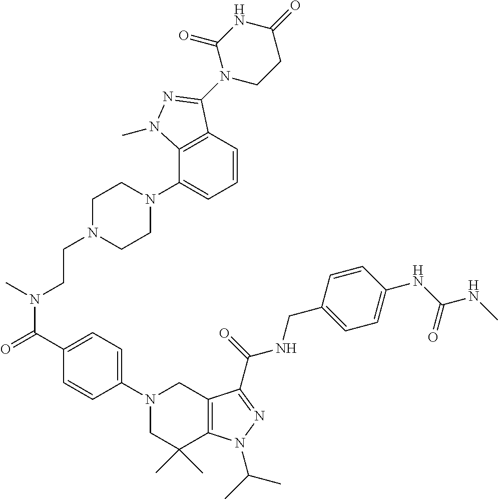 | 5-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7,7-dimethyl-N- (4-(3-methylureido)benzyl)- 4,5,6,7-tetrahydro-1H- pyrazolo[4,3-c]pyridine-3- carboxamide |
| VE188 | 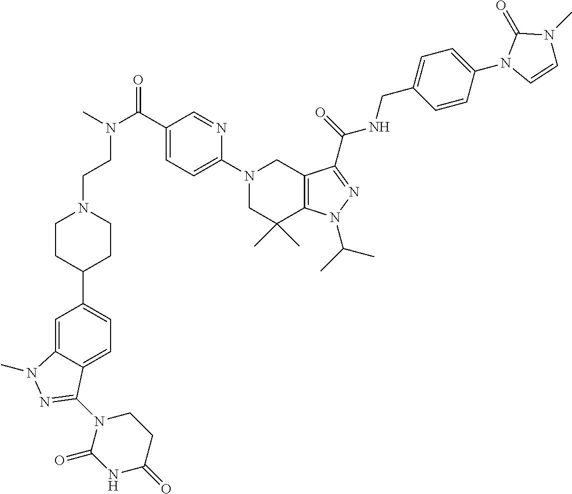 | 5-(5-((2-(4-(3-(2,4- dioxotetrahydropyrimidin-1(2H)- yl)-1-methyl-1H-indazol-6- yl)piperidin-1- yl)ethyl)(methyl)carbamoyl)pyridin- 2-yl)-1-isopropyl-7,7-dimethyl- N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE189 | 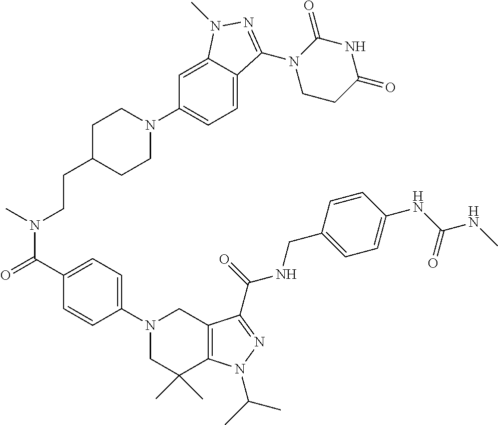 | 5-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7,7-dimethyl-N- (4-(3-methylureido)benzyl)- 4,5,6,7-tetrahydro-1H- pyrazolo[4,3-c]pyridine-3- carboxamide |
| VE190 | 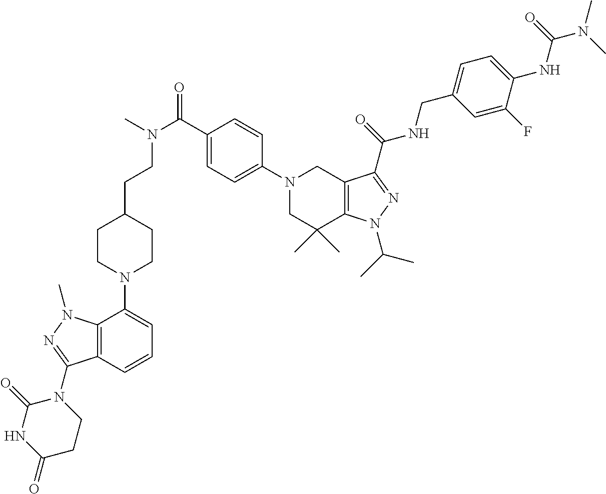 [SMILES: CC(C)n1nc(C(=O)NCc2ccc(NC(=O)N(C)C)c(F)c2)c2c1C(C)(C)CN(c1ccc(C(=O)N(C)CCC3CCN(c4cccc5c(N6CCC(=O)NC6=O)nn(C)c45)CC3)cc1)C2] | N-(4-(3,3-dimethylureido)-3- fluorobenzyl)-5-(5-((2-(1-(3-(2,4- dioxotetrahydropyrimidin-1(2H)- yl)-1-methyl-1H-indazol-7- yl)piperidin-4- yl)ethyl)(methyl)carbamoyl)pyridin- 2-yl)-1-isopropyl-7,7-dimethyl- 4,5,6,7-tetrahydro-1H- pyrazolo[4,3-c]pyridine-3- carboxamide |
| VE191 | 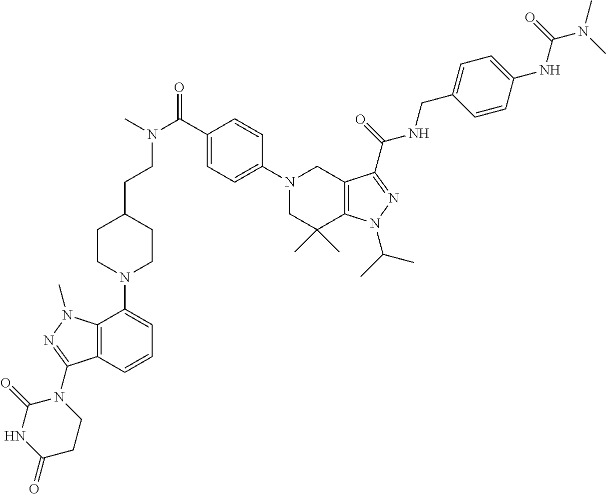 [SMILES: CC(C)n1nc(C(=O)NCc2ccc(NC(=O)N(C)C)cc2)c2c1C(C)(C)CN(c1ccc(C(=O)N(C)CCC3CCN(c4cccc5c(N6CCC(=O)NC6=O)nn(C)c45)CC3)cc1)C2] | N-(4-(3,3- dimethylureido)benzyl)-5-(4-((2- (1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE192 | 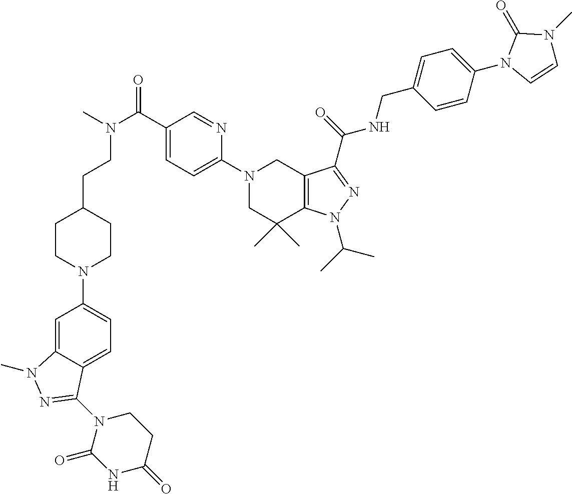 | 5-(5-((2-(1-(3-(2,4- dioxotetrahydropyrimidin-1(2H)- yl)-1-methyl-1H-indazol-6- yl)piperidin-4- yl)ethyl)(methyl)carbamoyl)pyridin- 2-yl)-1-isopropyl-7,7-dimethyl- N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE194 | 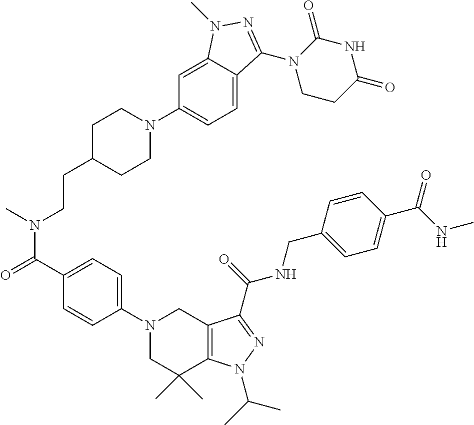 | 5-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 1-isopropyl-7,7-dimethyl-N- (4-(methylcarbamoyl)benzyl)- 4,5,6,7-tetrahydro-1H- pyrazolo[4,3-c]pyridine-3- carboxamide |
| VE195 | 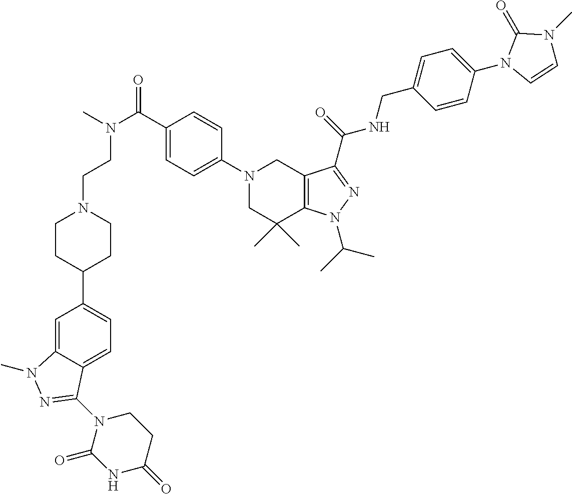 | 5-(4-((2-(4-(3-(2,4- dioxotetrahydropyrimidin-1(2H)- yl)-1-methyl-1H-indazol-6- yl)piperidin-1- yl)ethyl)(methyl)carbamoyl)phenyl)- 1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE196 | 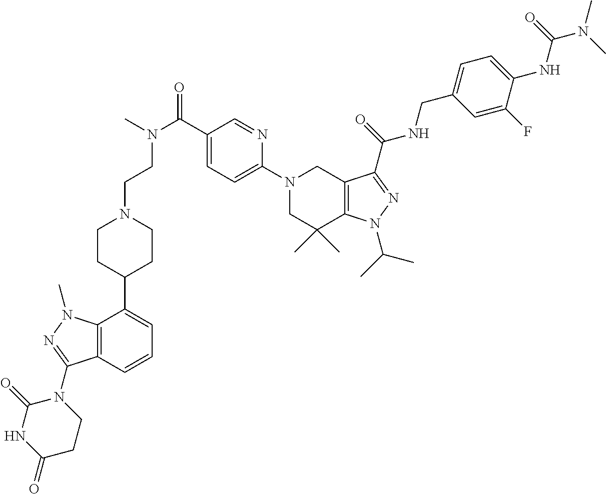 [SMILES: CC(C)n1nc(C(=O)NCc2ccc(NC(=O)N(C)C)c(F)c2)c2c1C(C)(C)CN(c1ccc(C(=O)N(C)CCN3CCC(c4cccc5c(N6CCC(=O)NC6=O)nn(C)c45)CC3)cn1)C2] | N-(4-(3,3-dimethylureido)-3- fluorobenzyl)-5-(5-((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-1-isopropyl-7,7-dimethyl- 4,5,6,7-tetrahydro-1H- pyrazolo[4,3-c]pyridine-3- carboxamide |
| VE197 | 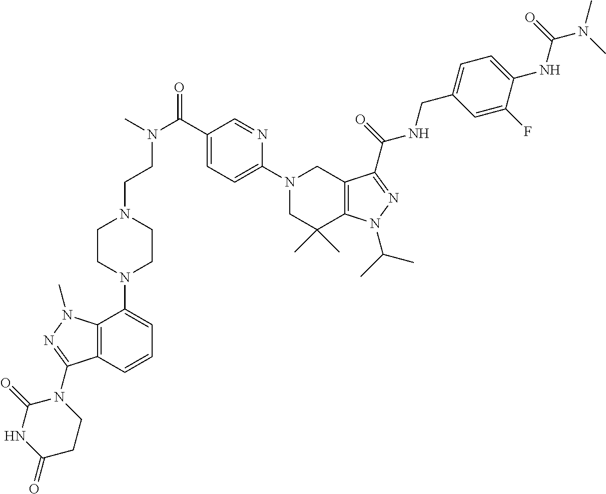 [SMILES: CC(C)n1nc(C(=O)NCc2ccc(NC(=O)N(C)C)c(F)c2)c2c1C(C)(C)CN(c1ccc(C(=O)N(C)CCN3CCN(c4cccc5c(N6CCC(=O)NC6=O)nn(C)c45)CC3)cn1)C2] | 5-(5-((2-(4-(3-(2,4- dioxotetrahydropyrimidin-1(2H)- yl)-1-methyl-1H-indazol-7- yl)piperazin-1- yl)ethyl)(methyl)carbamoyl)pyridin- 2-yl)-1-isopropyl-7,7-dimethyl- N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE198 | 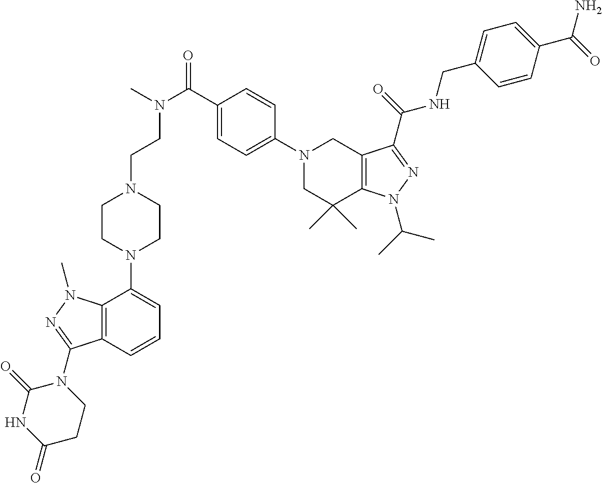 [SMILES: CC(C)n1nc(C(=O)NCc2ccc(C(N)=O)cc2)c2c1C(C)(C)CN(c1ccc(C(=O)N(C)CCN3CCN(c4cccc5c(N6CCC(=O)NC6=O)nn(C)c45)CC3)cc1)C2] | N-(4-carbamoylbenzyl)-5-(4-((2- (4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE199 | 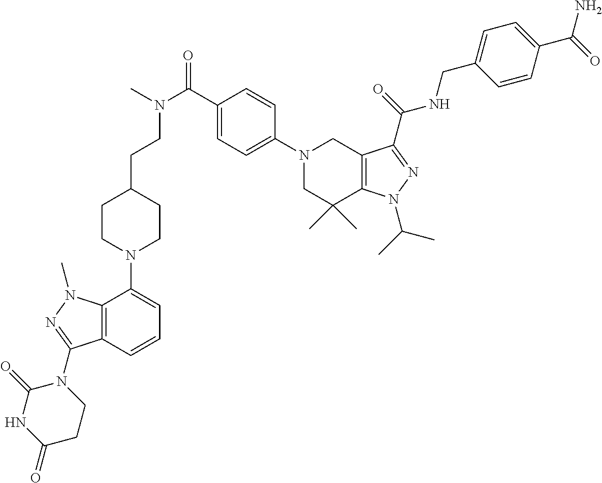 [SMILES: CC(C)n1nc(C(=O)NCc2ccc(C(N)=O)cc2)c2c1C(C)(C)CN(c1ccc(C(=O)N(C)CCC3CCN(c4cccc5c(N6CCC(=O)NC6=O)nn(C)c45)CC3)cc1)C2] | N-(4-carbamoylbenzyl)-5-(4-((2- (1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE203 | 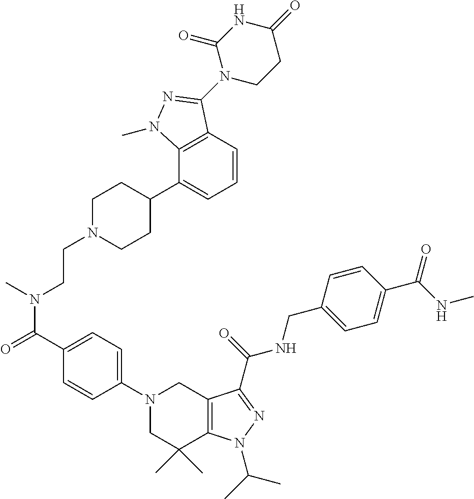 | 5-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7,7-dimethyl-N- (4-(methylcarbamoyl)benzyl)- 4,5,6,7-tetrahydro-1H- pyrazolo[4,3-c]pyridine-3- carboxamide |
| VE204 | 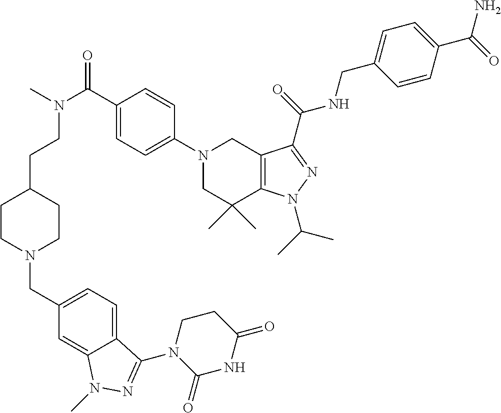 [SMILES: CC(C)n1nc(C(=O)NCc2ccc(C(N)=O)cc2)c2c1C(C)(C)CN(c1ccc(C(=O)N(C)CCC3CCN(Cc4ccc5c(N6CCC(=O)NC6=O)nn(C)c5c4)CC3)cc1)C2] | N-(4-carbamoylbenzyl)-5-(4-((2- (1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE206 | 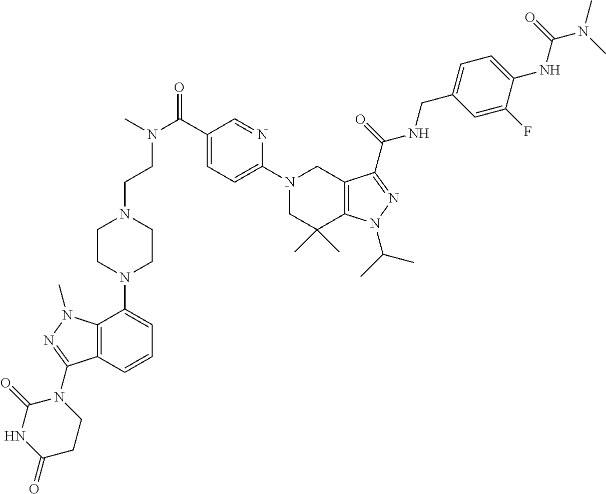 [SMILES: CC(C)n1nc(C(=O)NCc2ccc(NC(=O)N(C)C)c(F)c2)c2c1C(C)(C)CN(c1ccc(C(=O)N(C)CCN3CCN(c4cccc5c(N6CCC(=O)NC6=O)nn(C)c45)CC3)cn1)C2] | N-(4-(3,3-dimethylureido)-3- fluorobenzyl)-5-(5-((2-(4-(3-(2,4- dioxotetrahydropyrimidin-1(2H)- yl)-1-methyl-1H-indazol-7- yl)piperazin-1- yl)ethyl)(methyl)carbamoyl)pyridin- 2-yl)-1-isopropyl-7,7-dimethyl- 4,5,6,7-tetrahydro-1H- pyrazolo[4,3-c]pyridine-3- carboxamide |
| VE207 | 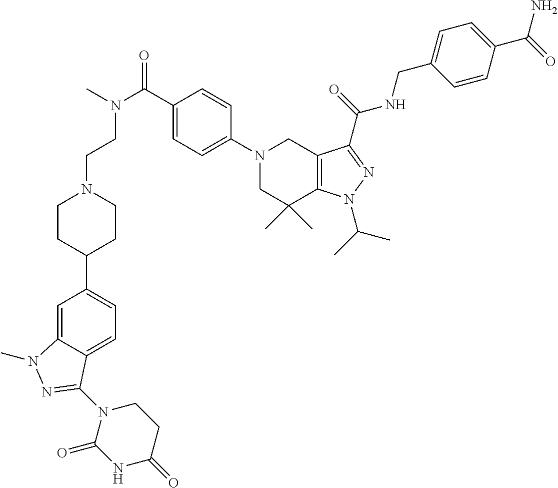 [SMILES: CC(C)n1nc(C(=O)NCc2ccc(C(N)=O)cc2)c2c1C(C)(C)CN(c1ccc(C(=O)N(C)CCN3CCC(c4ccc5c(N6CCC(=O)NC6=O)nn(C)c5c4)CC3)cc1)C2] | N-(4-carbamoylbenzyl)-5-(4-((2- (4-(3-(2,4- dioxotetrahydropyrimidin-1(2H)- yl)-1-methyl-1H-indazol-6- yl)piperidin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE209 | 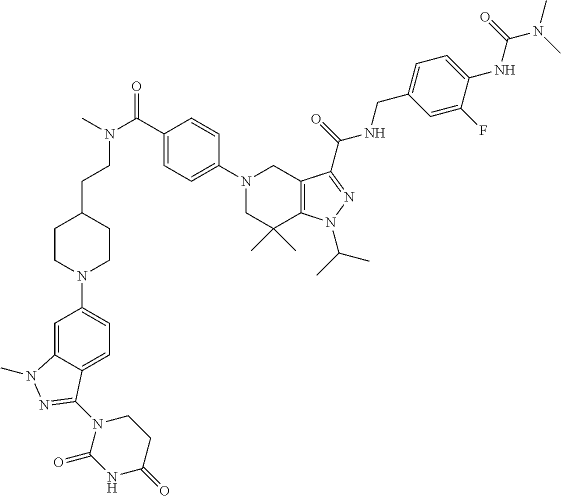 | N-(4-(3,3-dimethylureido)-3- fluorobenzyl)-5-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE210 | 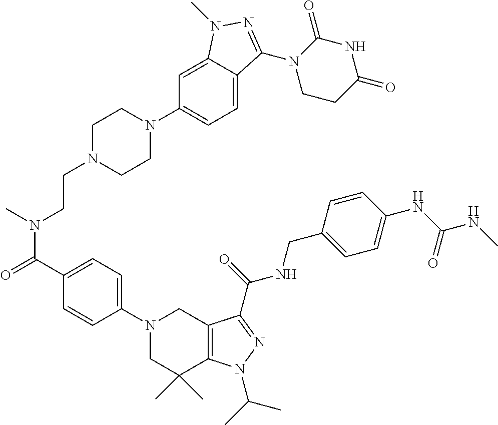 | 5-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 1-isopropyl-7,7-dimethyl-N- (4-(3-methylureido)benzyl)- 4,5,6,7-tetrahydro-1H- pyrazolo[4,3-c]pyridine-3- carboxamide |
| VE211 | 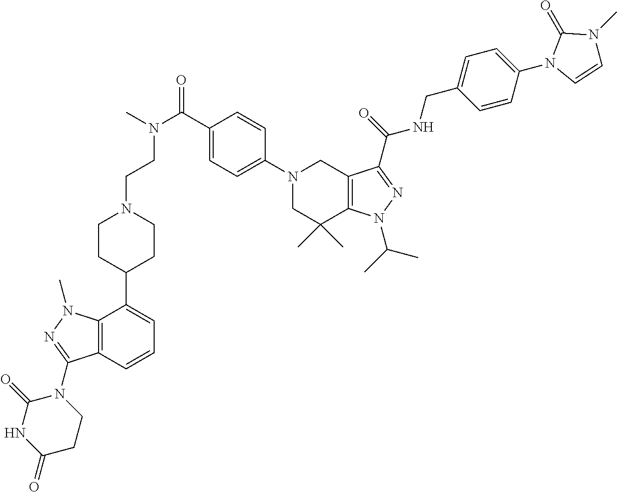 [SMILES: CC(C)n1nc(C(=O)NCc2ccc(-n3ccn(C)c3=O)cc2)c2c1C(C)(C)CN(c1ccc(C(=O)N(C)CCN3CCC(c4cccc5c(N6CCC(=O)NC6=O)nn(C)c45)CC3)cc1)C2] | 5-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE212 | 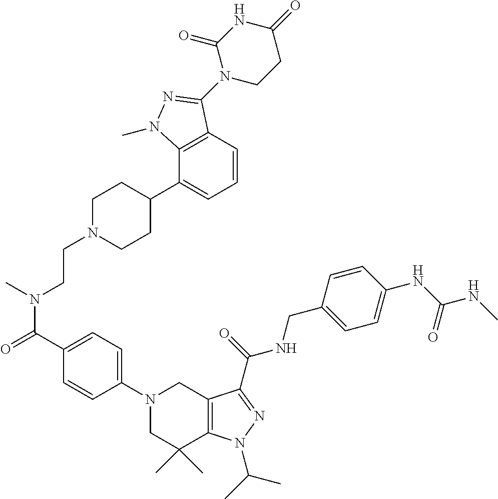 | 5-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7,7-dimethyl-N- (4-(3-methylureido)benzyl)- 4,5,6,7-tetrahydro-1H- pyrazolo[4,3-c]pyridine-3- carboxamide |
| VE213 | 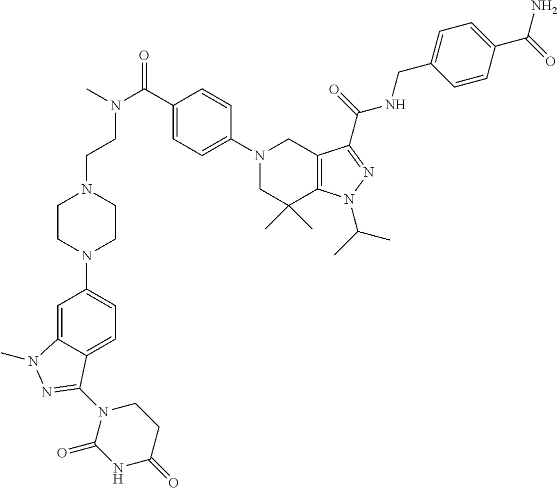 [SMILES: CC(C)n1nc(C(=O)NCc2ccc(C(N)=O)cc2)c2c1C(C)(C)CN(c1ccc(C(=O)N(C)CCN3CCN(c4ccc5c(N6CCC(=O)NC6=O)nn(C)c5c4)CC3)cc1)C2] | N-(4-carbamoylbenzyl)-5-(4-((2- (4-(3-(2,4- dioxotetrahydropyrimidin-1(2H)- yl)-1-methyl-1H-indazol-6- yl)piperazin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE214 | 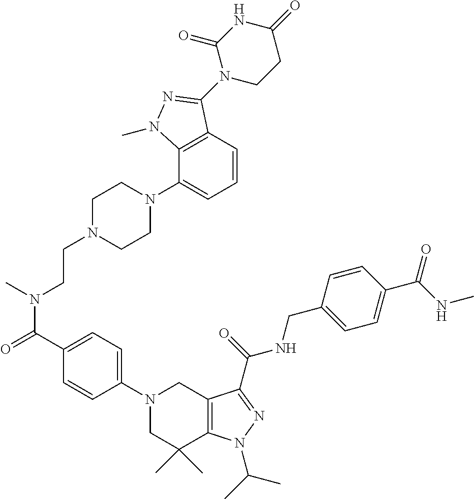 | 5-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7,7-dimethyl-N- (4-(methylcarbamoyl)benzyl)- 4,5,6,7-tetrahydro-1H- pyrazolo[4,3-c]pyridine-3- carboxamide |
| VE215 | 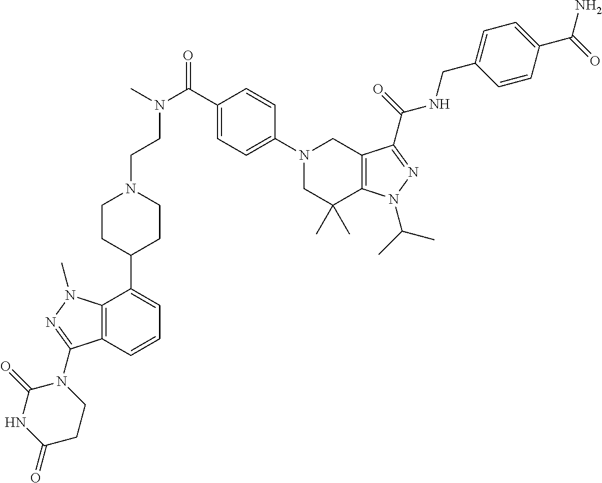 [SMILES: CC(C)n1nc(C(=O)NCc2ccc(C(N)=O)cc2)c2c1C(C)(C)CN(c1ccc(C(=O)N(C)CCN3CCC(c4cccc5c(N6CCC(=O)NC6=O)nn(C)c45)CC3)cc1)C2] | N-(4-carbamoylbenzyl)-5-(4-((2- (4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7,7-dimethyl- 4,5,6,7-tetrahydro-1H- pyrazolo[4,3-c]pyridine-3- carboxamide |
| VE216 | 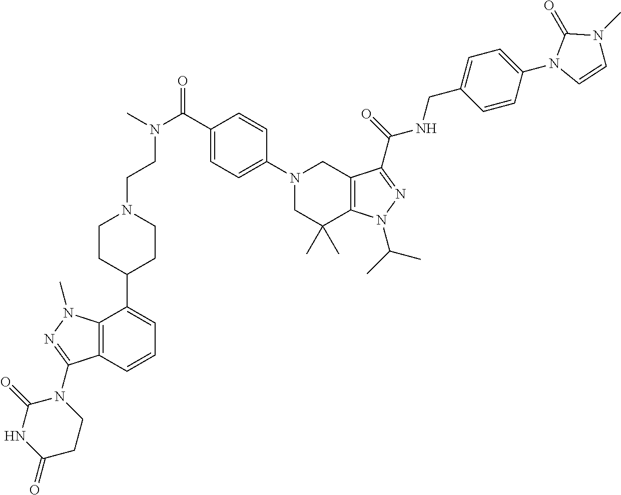 [SMILES: CC(C)n1nc(C(=O)NCc2ccc(-n3ccn(C)c3=O)cc2)c2c1C(C)(C)CN(c1ccc(C(=O)N(C)CCN3CCC(c4cccc5c(N6CCC(=O)NC6=O)nn(C)c45)CC3)cc1)C2] | 5-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE217 | 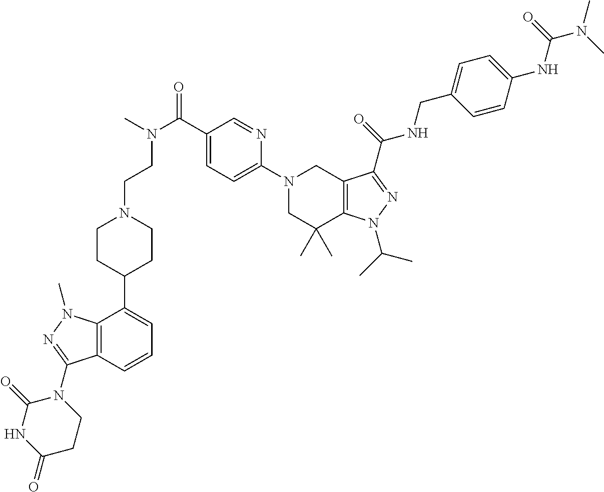 [SMILES: CC(C)n1nc(C(=O)NCc2ccc(NC(=O)N(C)C)cc2)c2c1C(C)(C)CN(c1ccc(C(=O)N(C)CCN3CCC(c4cccc5c(N6CCC(=O)NC6=O)nn(C)c45)CC3)cn1)C2] | N-(4-(3,3- dimethylureido)benzyl)-5-(5-((2- (4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-1-isopropyl-7,7-dimethyl- 4,5,6,7-tetrahydro-1H- pyrazolo[4,3-c]pyridine-3- carboxamide |
| VE218 | 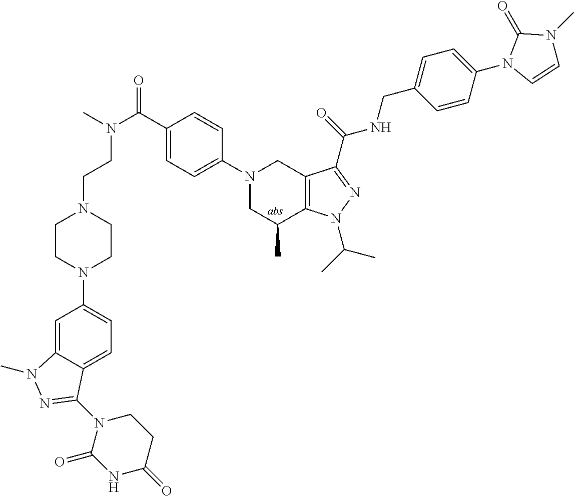 | (S)-5-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE220 | 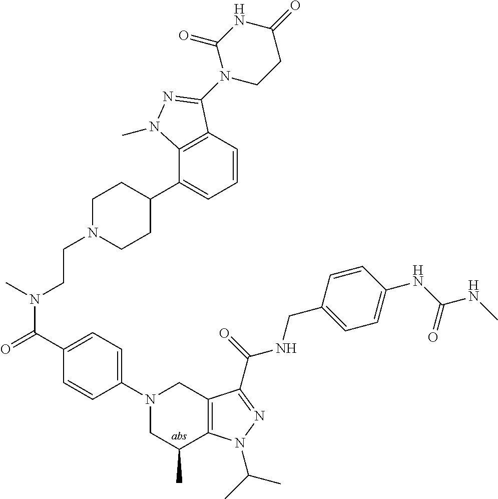 | (S)-5-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7-methyl-N-(4-(3- methylureido)benzyl)-4,5,6,7- tetrahydro-1H-pyrazolo[4,3- c]pyridine-3-carboxamide |
| VE221 | 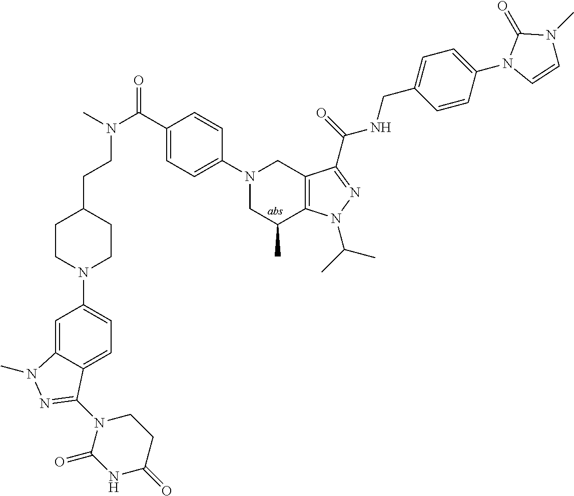 | (S)-5-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE223 | 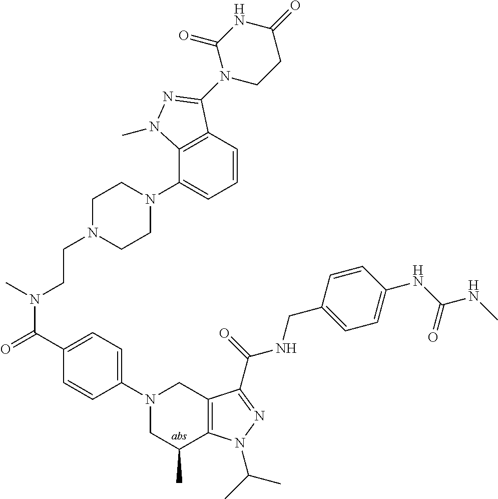 | (S)-5-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7-methyl-N-(4-(3- methylureido)benzyl)-4,5,6,7- tetrahydro-1H-pyrazolo[4,3- c]pyridine-3-carboxamide |
| VE224 | 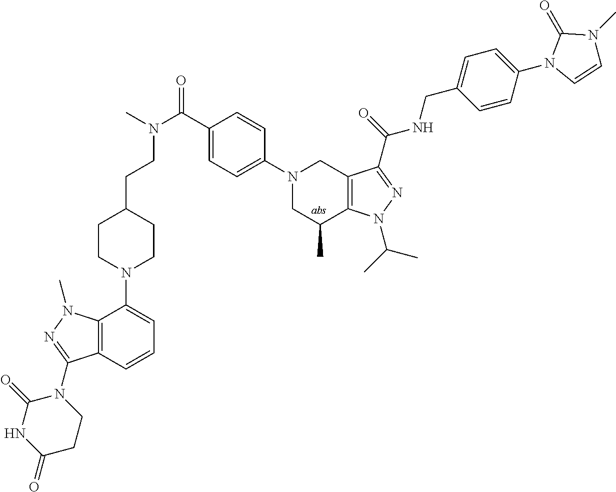 [SMILES: CC(C)n1nc(C(=O)NCc2ccc(-n3ccn(C)c3=O)cc2)c2c1[C@@H](C)CN(c1ccc(C(=O)N(C)CCC3CCN(c4cccc5c(N6CCC(=O)NC6=O)nn(C)c45)CC3)cc1)C2] | (S)-5-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE225 | 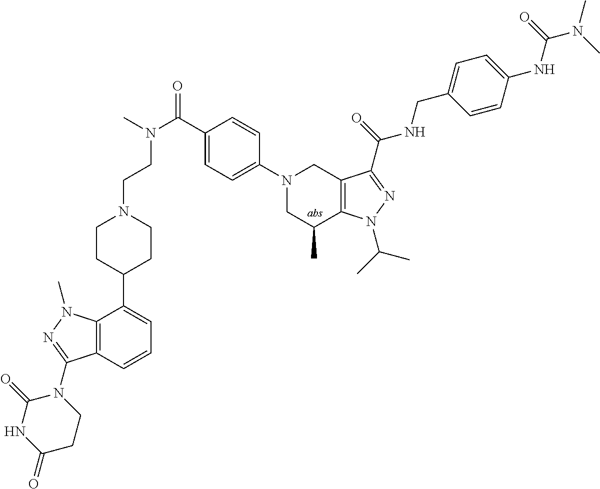 [SMILES: CC(C)n1nc(C(=O)NCc2ccc(NC(=O)N(C)C)cc2)c2c1[C@@H](C)CN(c1ccc(C(=O)N(C)CCN3CCC(c4cccc5c(N6CCC(=O)NC6=O)nn(C)c45)CC3)cc1)C2] | (S)-N-(4-(3,3- dimethylureido)benzyl)-5-(5-((2- (4-(3-(2,4- dioxotetrahydropyrimidin-1(2H)- yl)-1-methyl-1H-indazol-7- yl) piperidin-1- yl)ethyl)(methyl)carbamoyl)pyridin- 2-yl)-1-isopropyl-7-methyl- 4,5,6,7-tetrahydro-1H- pyrazolo[4,3-c]pyridine-3- carboxamide |
| VE226 | 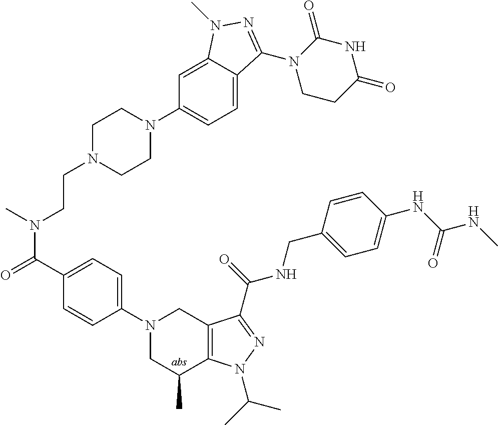 | (S)-5-(4-((2-(4-(3-(2,4- dioxotetrahydropyrimidin-1(2H)- yl)-1-methyl-1H-indazol-6- yl)piperazin-1- yl)ethyl)(methyl)carbamoyl)phenyl)- 1-isopropyl-7-methyl-N-(4-(3- methylureido)benzyl)-4,5,6,7- tetrahydro-1H-pyrazolo[4,3- c]pyridine-3-carboxamide |
| VE227 | 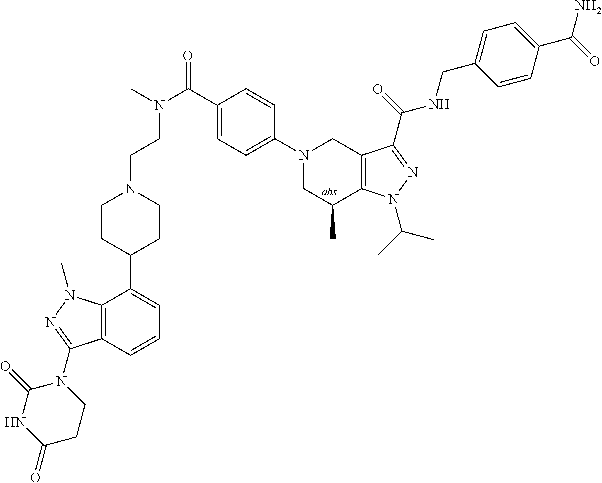 [SMILES: CC(C)n1nc(C(=O)NCc2ccc(C(N)=O)cc2)c2c1[C@@H](C)CN(c1ccc(C(=O)N(C)CCN3CCC(c4cccc5c(N6CCC(=O)NC6=O)nn(C)c45)CC3)cc1)C2] | (S)-N-(4-carbamoylbenzyl)-5-(4- ((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE228 | 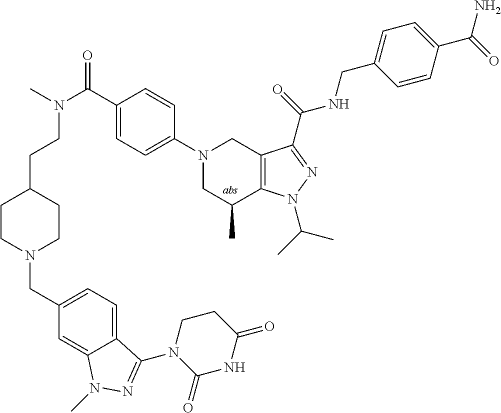 [SMILES: CC(C)n1nc(C(=O)NCc2ccc(C(N)=O)cc2)c2c1[C@@H](C)CN(c1ccc(C(=O)N(C)CCC3CCN(Cc4ccc5c(N6CCC(=O)NC6=O)nn(C)c5c4)CC3)cc1)C2] | (S)-N-(4-carbamoylbenzyl)-5-(4- ((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE229 | 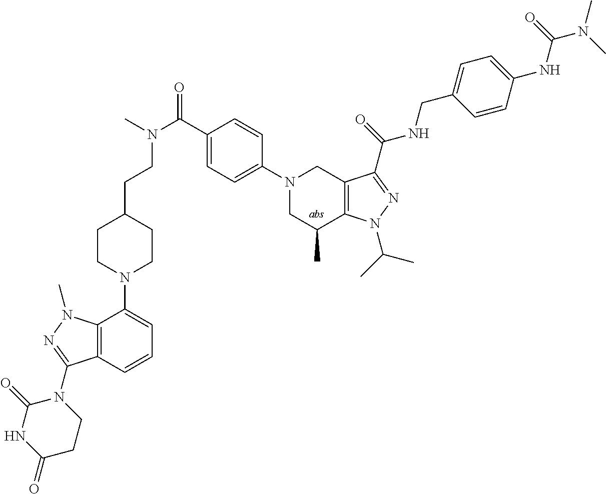 [SMILES: CC(C)n1nc(C(=O)NCc2ccc(NC(=O)N(C)C)cc2)c2c1[C@@H](C)CN(c1ccc(C(=O)N(C)CCC3CCN(c4cccc5c(N6CCC(=O)NC6=O)nn(C)c45)CC3)cc1)C2] | (S)-N-(4-(3,3- dimethylureido)benzyl)-5-(4-((2- (1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE230 | 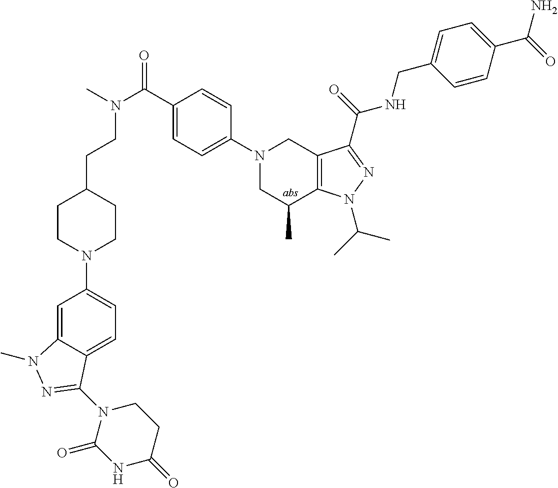 [SMILES: CC(C)n1nc(C(=O)NCc2ccc(C(N)=O)cc2)c2c1[C@@H](C)CN(c1ccc(C(=O)N(C)CCC3CCN(c4ccc5c(N6CCC(=O)NC6=O)nn(C)c5c4)CC3)cc1)C2] | (S)-N-(4-carbamoylbenzyl)-5-(4- ((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE231 | 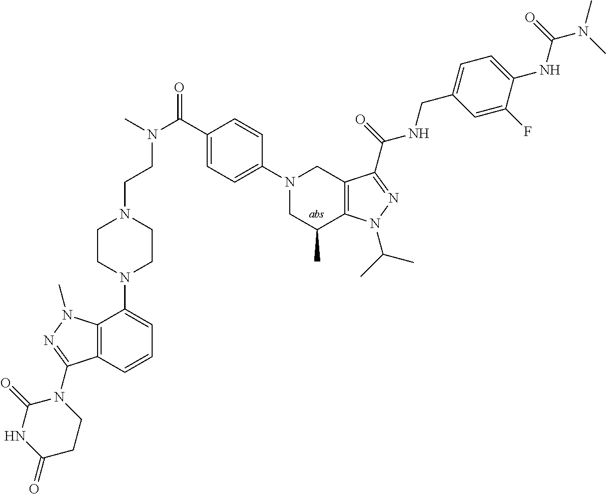 [SMILES: CC(C)n1nc(C(=O)NCc2ccc(NC(=O)N(C)C)c(F)c2)c2c1[C@@H](C)CN(c1ccc(C(=O)N(C)CCN3CCN(c4cccc5c(N6CCC(=O)NC6=O)nn(C)c45)CC3)cc1)C2] | (S)-N-(4-(3,3-dimethylureido)-3- fluorobenzyl)-5-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE233 | 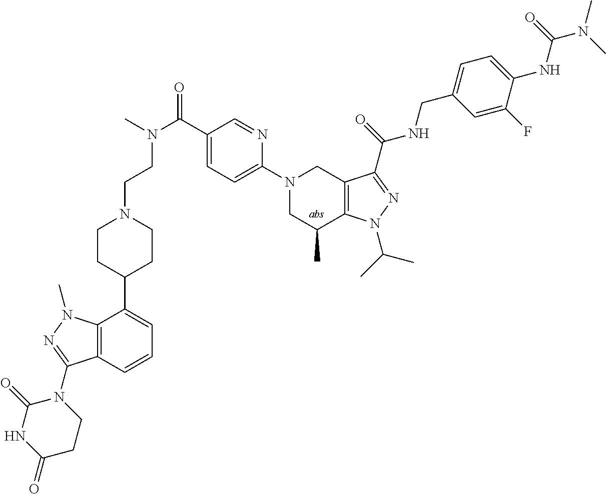 [SMILES: CC(C)n1nc(C(=O)NCc2ccc(NC(=O)N(C)C)c(F)c2)c2c1[C@@H](C)CN(c1ccc(C(=O)N(C)CCN3CCC(c4cccc5c(N6CCC(=O)NC6=O)nn(C)c45)CC3)cn1)C2] | (S)-N-(4-(3,3-dimethylureido)-3- fluorobenzyl)-5-(5-((2-(4-(3-(2,4- dioxotetrahydropyrimidin-1(2H)- yl)-1-methyl-1H-indazol-7- yl)piperidin-1- yl)ethyl)(methyl)carbamoyl)pyridin- 2-yl)-1-isopropyl-7-methyl- 4,5,6,7-tetrahydro-1H- pyrazolo[4,3-c]pyridine-3- carboxamide |
| VE234 | 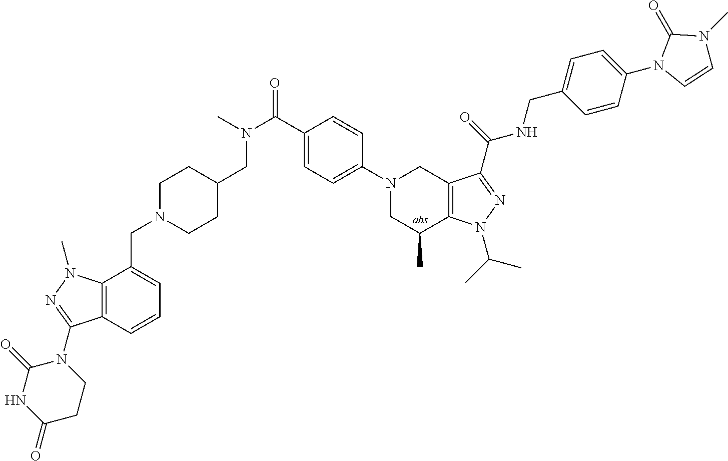 [SMILES: CC(C)n1nc(C(=O)NCc2ccc(-n3ccn(C)c3=O)cc2)c2c1[C@@H](C)CN(c1ccc(C(=O)N(C)CC3CCN(Cc4cccc5c(N6CCC(=O)NC6=O)nn(C)c45)CC3)cc1)C2] | (S)-5-(4-(((1-((3-(2,4- dioxotetrahydropyrimidin-1(2H)- yl)-1-methyl-1H-indazol-7- yl)methyl)piperidin-4- yl)methyl)(methyl)carbamoyl) phenyl)-1-isopropyl-7-methyl-N-(4- (3-methyl-2-oxo-2,3-dihydro-1H- imidazol-1-yl)benzyl)-4,5,6,7- tetrahydro-1H-pyrazolo[4,3- c]pyridine-3-carboxamide |
| VE236 | 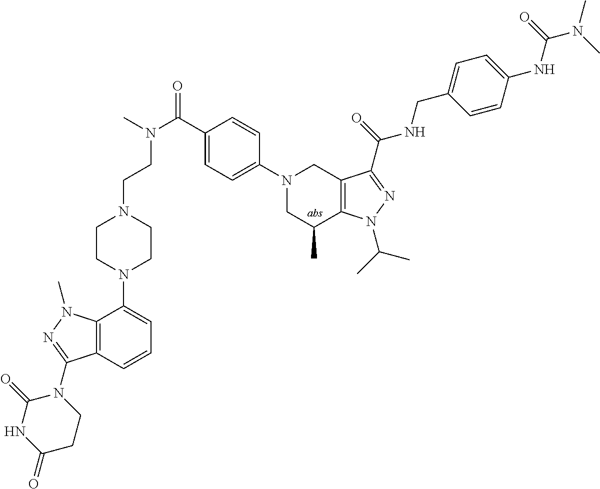 [SMILES: CC(C)n1nc(C(=O)NCc2ccc(NC(=O)N(C)C)cc2)c2c1[C@@H](C)CN(c1ccc(C(=O)N(C)CCN3CCN(c4cccc5c(N6CCC(=O)NC6=O)nn(C)c45)CC3)cc1)C2] | (S)-N-(4-(3,3- dimethylureido)benzyl)-5-(4-((2- (4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE237 | 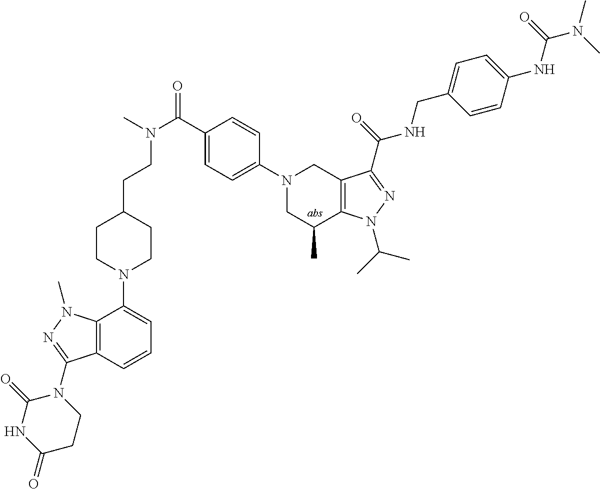 [SMILES: CC(C)n1nc(C(=O)NCc2ccc(NC(=O)N(C)C)cc2)c2c1[C@@H](C)CN(c1ccc(C(=O)N(C)CCC3CCN(c4cccc5c(N6CCC(=O)NC6=O)nn(C)c45)CC3)cc1)C2] | (S)-N-(4-(3,3-dimethylureido)-3- fluorobenzyl)-5-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE238 | 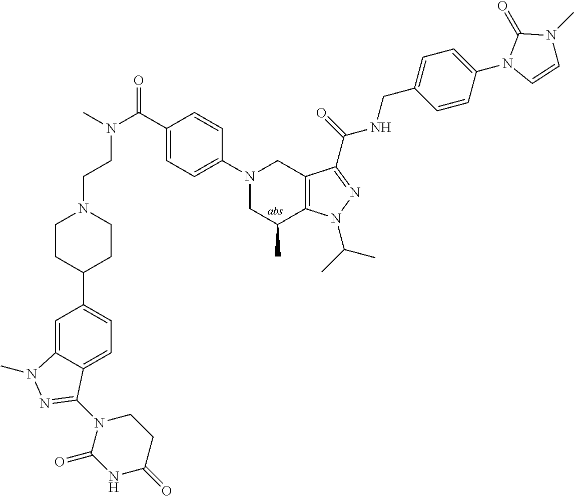 | (S)-5-(4-((2-(4-(3-(2,4- dioxotetrahydropyrimidin-1(2H)- yl)-1-methyl-1H-indazol-6- yl)piperidin-1- yl)ethyl)(methyl)carbamoyl)phenyl)- 1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE239 | 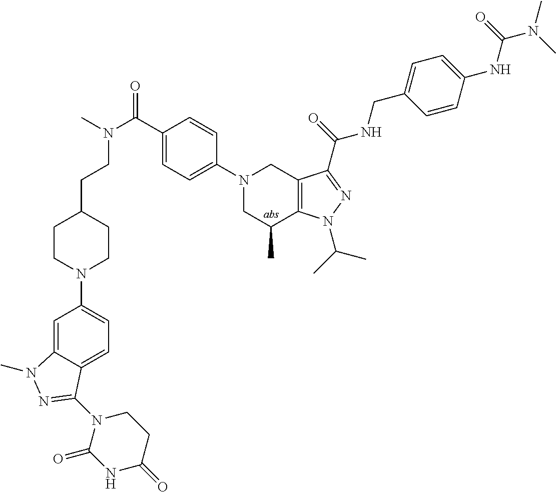 | (S)-N-(4-(3,3- dimethylureido)benzyl)-5-(4-((2- (1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE240 | 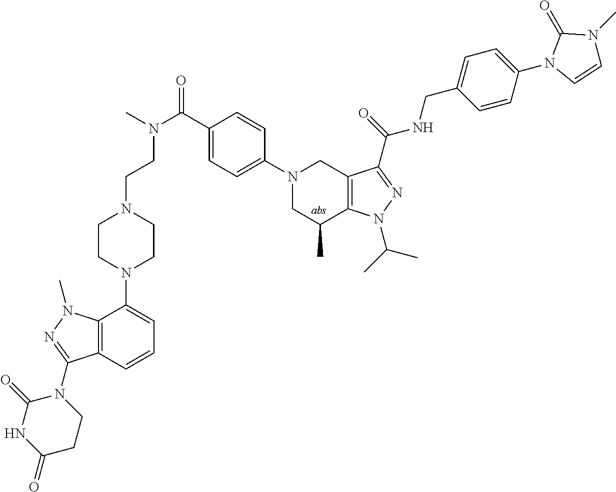 [SMILES: CC(C)n1nc(C(=O)NCc2ccc(-n3ccn(C)c3=O)cc2)c2c1[C@@H](C)CN(c1ccc(C(=O)N(C)CCN3CCN(c4cccc5c(N6CCC(=O)NC6=O)nn(C)c45)CC3)cc1)C2] | (S)-5-(4-((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE241 | 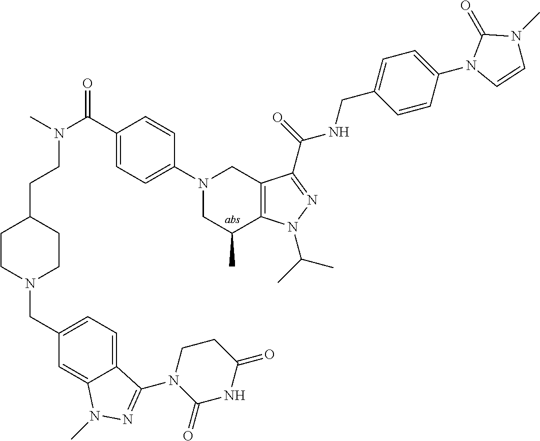 [SMILES: CC(C)n1nc(C(=O)NCc2ccc(-n3ccn(C)c3=O)cc2)c2c1[C@@H](C)CN(c1ccc(C(=O)N(C)CCC3CCN(Cc4ccc5c(N6CCC(=O)NC6=O)nn(C)c5c4)CC3)cc1)C2] | (S)-5-(4-((2-(1-((3-(2,4- dioxotetrahydropyrimidin-1(2H)- yl)-1-methyl-1H-indazol-6- yl)methyl)piperidin-4- yl)ethyl)(methyl)carbamoyl)phenyl)- 1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE242 | 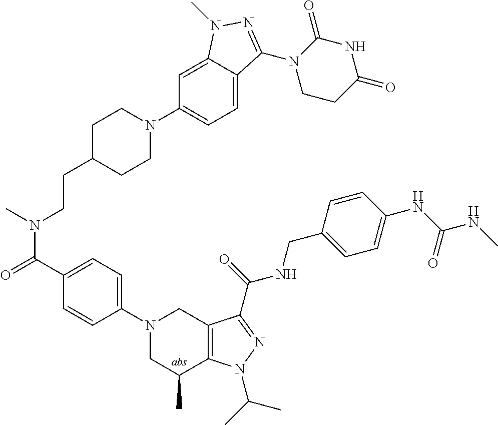 | (S)-5-(4-((2-(1-(3-(2,4- dioxotetrahydropyrimidin-1(2H)- yl)-1-methyl-1H-indazol-6- yl)piperidin-4- yl)ethyl)(methyl)carbamoyl)phenyl)- 1-isopropyl-7-methyl-N-(4-(3- methylureido)benzyl)-4,5,6,7- tetrahydro-1H-pyrazolo[4,3- c]pyridine-3-carboxamide |
| VE243 | 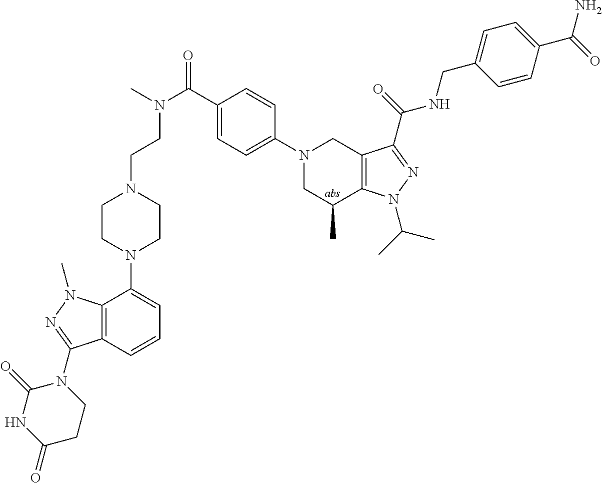 [SMILES: CC(C)n1nc(C(=O)NCc2ccc(C(N)=O)cc2)c2c1[C@@H](C)CN(c1ccc(C(=O)N(C)CCN3CCN(c4cccc5c(N6CCC(=O)NC6=O)nn(C)c45)CC3)cc1)C2] | (S)-N-(4-carbamoylbenzyl)-5-(4- ((2-(4-(3-(2,4- dioxotetrahydropyrimidin-1(2H)- yl)-1-methyl-1H-indazol-7- yl)piperazin-1- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE244 | 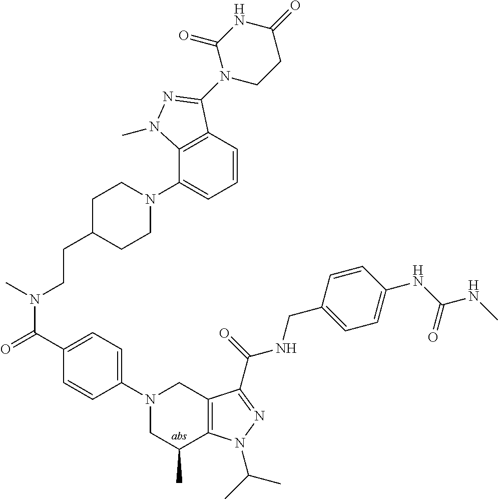 | (S)-5-(4-((2-(1-(3-(2,4- dioxotetrahydropyrimidin-1(2H)- yl)-1-methyl-1H-indazol-7- yl)piperidin-4- yl)ethyl)(methyl)carbamoyl)phenyl)- 1-isopropyl-7-methyl-N-(4-(3- methylureido)benzyl)-4,5,6,7- tetrahydro-1H-pyrazolo[4,3- c]pyridine-3-carboxamide |
| VE247 | 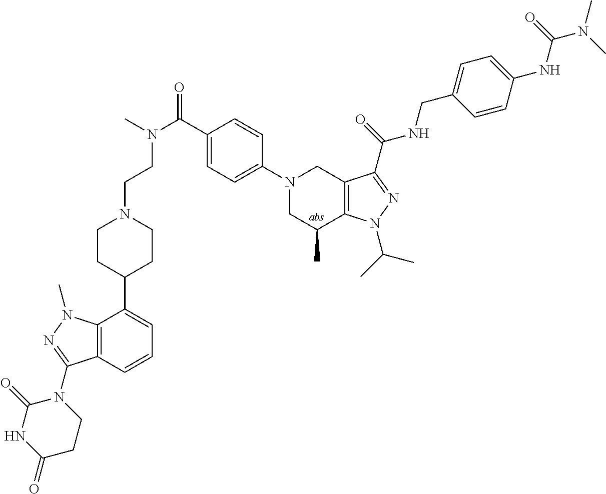 [SMILES: CC(C)n1nc(C(=O)NCc2ccc(NC(=O)N(C)C)cc2)c2c1[C@@H](C)CN(c1ccc(C(=O)N(C)CCN3CCC(c4cccc5c(N6CCC(=O)NC6=O)nn(C)c45)CC3)cc1)C2] | (S)-N-(4-(3,3- dimethylureido)benzyl)-5-(4-((2- (4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE248 | 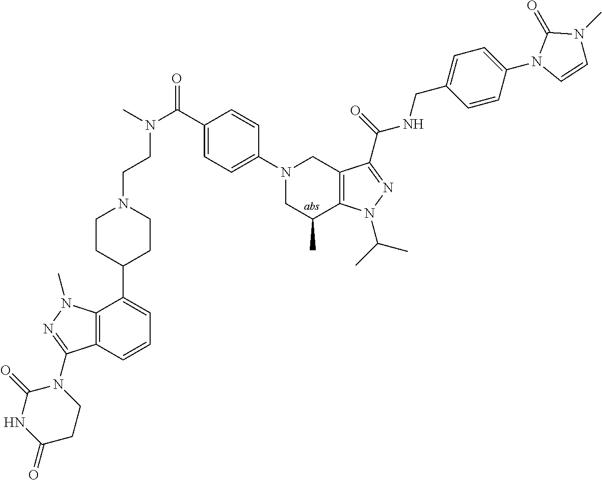 [SMILES: CC(C)n1nc(C(=O)NCc2ccc(-n3ccn(C)c3=O)cc2)c2c1[C@@H](C)CN(c1ccc(C(=O)N(C)CCN3CCC(c4cccc5c(N6CCC(=O)NC6=O)nn(C)c45)CC3)cc1)C2] | (S)-5-(4-((2-(4-(3-(2,4- dioxotetrahydropyrimidin-1(2H)- yl)-1-methyl-1H-indazol-7- yl)piperidin-1- yl)ethyl)(methyl)carbamoyl)phenyl)- 1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE250 | 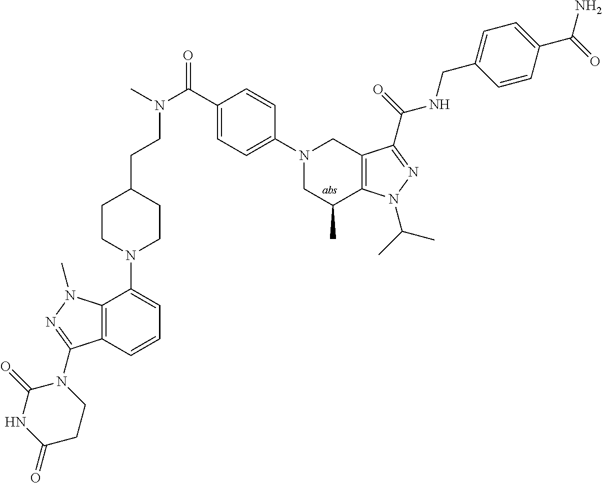 [SMILES: CC(C)n1nc(C(=O)NCc2ccc(C(N)=O)cc2)c2c1[C@@H](C)CN(c1ccc(C(=O)N(C)CCC3CCN(c4cccc5c(N6CCC(=O)NC6=O)nn(C)c45)CC3)cc1)C2] | (S)-N-(4-carbamoylbenzyl)-5-(4- ((2-(1-(3-(2,4- dioxotetrahydropyrimidin-1(2H)- yl)-1-methyl-1H-indazol-7- yl) piperidin-4- yl)ethyl)(methyl)carbamoyl)phenyl)- 1-isopropyl-7-methyl-4,5,6,7- tetrahydro-1H-pyrazolo[4,3- c]pyridine-3-carboxamide |
| VE251 | 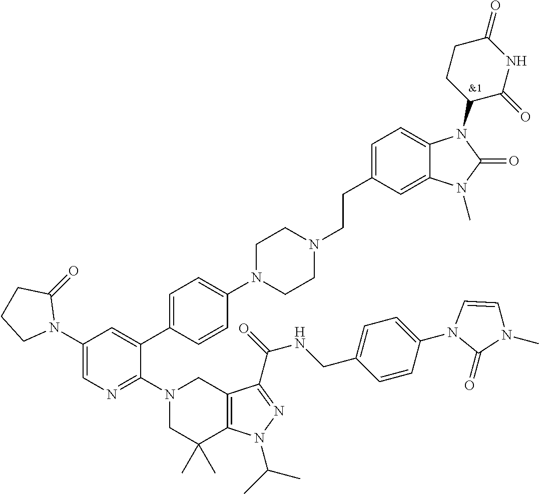 | rac-(R)-5-(3-(4-(4-(2-(1-(2,6- dioxopiperidin-3-yl)-3-methyl-2- oxo-2,3-dihydro-1H- benzo[d]imidazol-5- yl)ethyl)piperazin-1-yl)phenyl)-5- (2-oxopyrrolidin-1-yl)pyridin-2- yl)-1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE252 | 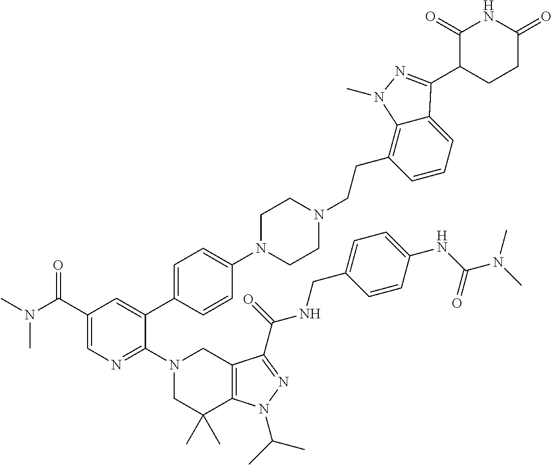 [SMILES: CC(C)n1nc(C(=O)NCc2ccc(NC(=O)N(C)C)cc2)c2c1C(C)(C)CN(c1ncc(C(=O)N(C)C)cc1-c1ccc(N3CCN(CCc4cccc5c(C6CCC(=O)NC6=O)nn(C)c45)CC3)cc1)C2] | 5-(5-(dimethylcarbamoyl)-3-(4- (4-(2-(3-(2,6-dioxopiperidin-3-yl)- 1-methyl-1H-indazol-7- yl)ethyl)piperazin-1- yl)phenyl)pyridin-2-yl)-N-(4-(3,3- dimethylureido)benzyl)-1- isopropyl-7,7-dimethyl-4,5,6,7- tetrahydro-1H-pyrazolo[4,3- c]pyridine-3-carboxamide |
| VE253 | 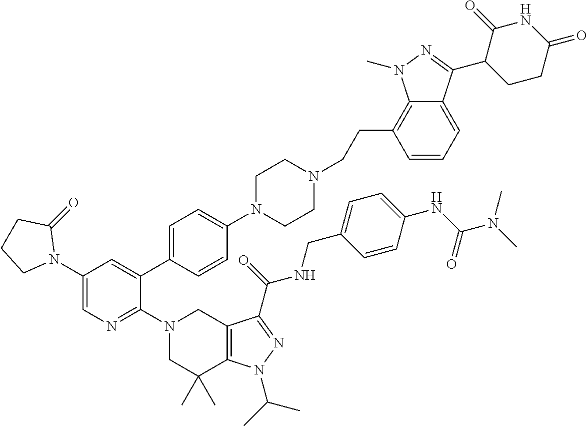 [SMILES: CC(C)n1nc(C(=O)NCc2ccc(NC(=O)N(C)C)cc2)c2c1C(C)(C)CN(c1ncc(N3CCCC3=O)cc1-c1ccc(N3CCN(CCc4cccc5c(C6CCC(=O)NC6=O)nn(C)c45)CC3)cc1)C2] | N-(4-(3,3- dimethylureido)benzyl)-5-(3-(4- (4-(2-(3-(2,6-dioxopiperidin-3-yl)- 1-methyl-1H-indazol-7- yl)ethyl)piperazin-1-yl)phenyl)-5- (2-oxopyrrolidin-1-yl)pyridin-2- yl)-1-isopropyl-7,7-dimethyl- 4,5,6,7-tetrahydro-1H- pyrazolo[4,3-c]pyridine-3- carboxamide |
| VE254 | 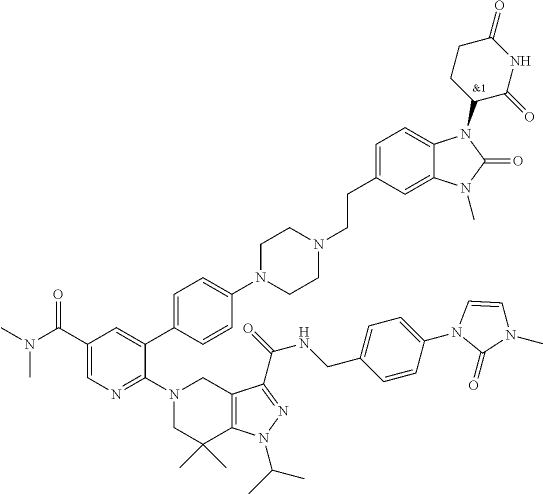 | rac-(R)-5-(5- (dimethylcarbamoyl)-3-(4-(4-(2- (1-(2,6-dioxopiperidin-3-yl)-3- methyl-2-oxo-2,3-dihydro-1H- benzo[d]imidazol-5- yl)ethyl)piperazin-1- yl)phenyl)pyridin-2-yl)-1- isopropyl-7,7-dimethyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE255 | 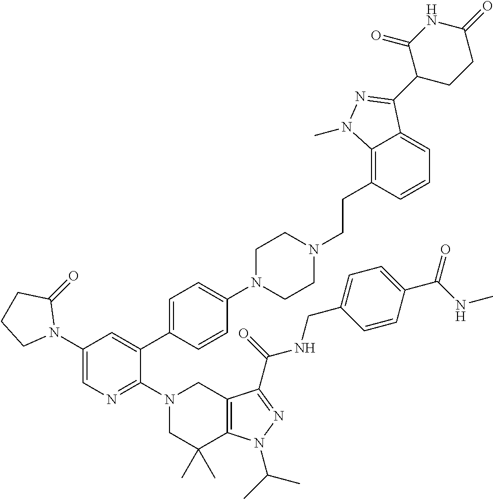 [SMILES: CNC(=O)c1ccc(CNC(=O)c2nn(C(C)C)c3c2CN(c2ncc(N4CCCC4=O)cc2-c2ccc(N4CCN(CCc5cccc6c(C7CCC(=O)NC7=O)nn(C)c56)CC4)cc2)CC3(C)C)cc1] | 5-(3-(4-(4-(2-(3-(2,6- dioxopiperidin-3-yl)-1-methyl-1H- indazol-7-yl)ethyl)piperazin-1- yl)phenyl)-5-(2-oxopyrrolidin-1- yl)pyridin-2-yl)-1-isopropyl-7,7- dimethyl-N-(4- (methylcarbamoyl)benzyl)- 4,5,6,7-tetrahydro-1H- pyrazolo[4,3-c]pyridine-3- carboxamide |
| VE256 | 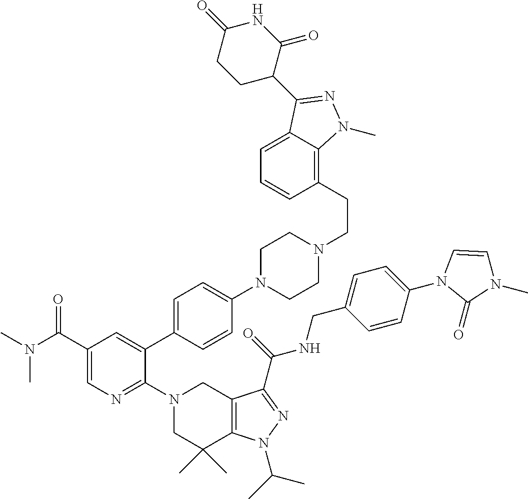 | 5-(5-(dimethylcarbamoyl)-3-(4- (4-(2-(3-(2,6-dioxopiperidin-3-yl)- 1-methyl-1H-indazol-7- yl)ethyl)piperazin-1- yl)phenyl)pyridin-2-yl)-1- isopropyl-7,7-dimethyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE257 | 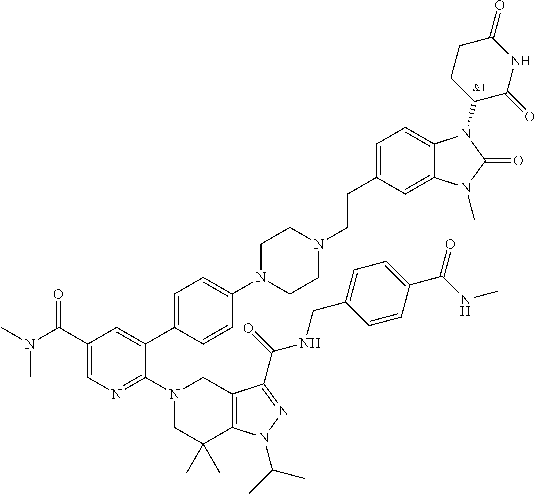 | rac-(R)-5-(5- (dimethylcarbamoyl)-3-(4-(4-(2- (1-(2,6-dioxopiperidin-3-yl)-3- methyl-2-oxo-2,3-dihydro-1H- benzo[d]imidazol-5- yl)ethyl)piperazin-1- yl)phenyl)pyridin-2-yl)-1- isopropyl-7,7-dimethyl-N-(4- (methylcarbamoyl)benzyl)- 4,5,6,7-tetrahydro-1H- pyrazolo[4,3-c]pyridine-3- carboxamide |
| VE258 | 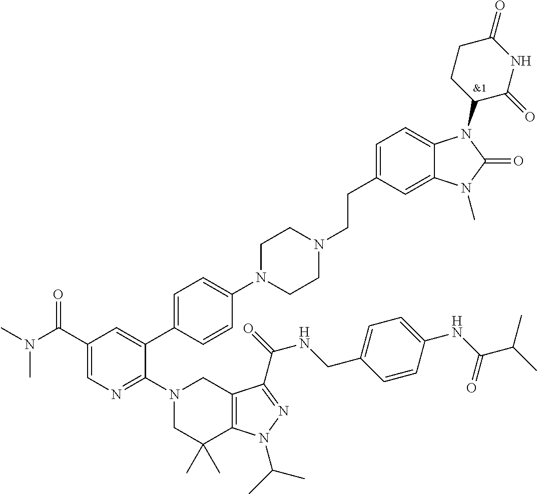 | rac-(R)-5-(5- (dimethylcarbamoyl)-3-(4-(4-(2- (1-(2,6-dioxopiperidin-3-yl)-3- methyl-2-oxo-2,3-dihydro-1H- benzo[d]imidazol-5- yl)ethyl)piperazin-1- yl)phenyl)pyridin-2-yl)-N-(4-(3,3- dimethylureido)benzyl)-1- isopropyl-7,7-dimethyl-4,5,6,7- tetrahydro-1H-pyrazolo[4,3- c]pyridine-3-carboxamide |
| VE259 | 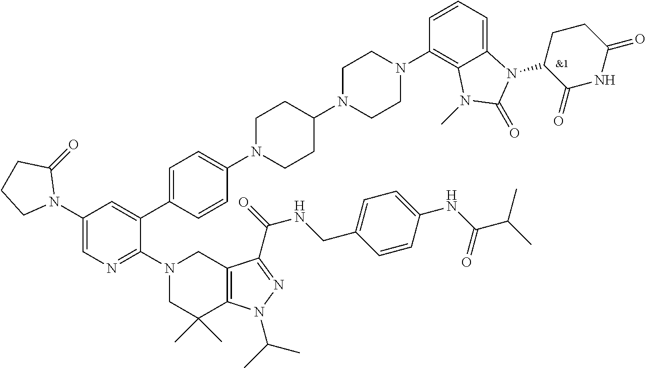 | rac-(R)-N-(4-(3,3- dimethylureido)benzyl)-5-(3-(4- (4-(4-(1-(2,6-dioxopiperidin-3-yl)- 3-methyl-2-oxo-2,3-dihydro-1H- benzo[d]imidazol-4-yl)piperazin- 1-yl)piperidin-1-yl)phenyl)-5-(2- oxopyrrolidin-1-yl)pyridin-2-yl)-1- isopropyl-7,7-dimethyl-4,5,6,7- tetrahydro-1H-pyrazolo[4,3- c]pyridine-3-carboxamide |
| VE260 | 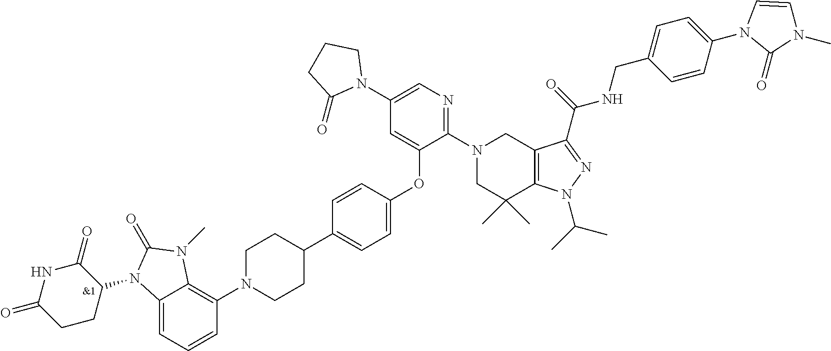 | rac-(R)-5-(3-(4-(1-(1-(2,6- dioxopiperidin-3-yl)-3-methyl-2- oxo-2,3-dihydro-1H- benzo[d]imidazol-4-yl)piperidin- 4-yl)phenoxy)-5-(2-oxopyrrolidin- 1-yl)pyridin-2-yl)-1-isopropyl-7,7- dimethyl-N-(4-(3-methyl-2-oxo- 2,3-dihydro-1H-imidazol-1- yl)benzyl)-4,5,6,7-tetrahydro-1H- pyrazolo[4,3-c]pyridine-3- carboxamide |
| VE261 | 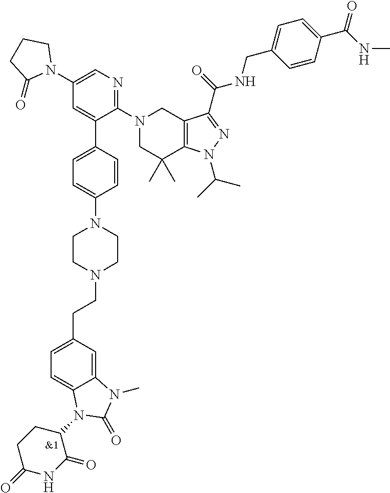 | rac-(R)-5-(3-(4-(4-(2-(1-(2,6- dioxopiperidin-3-yl)-3-methyl-2- oxo-2,3-dihydro-1H- benzo[d]imidazol-5- yl)ethyl)piperazin-1-yl)phenyl)-5- (2-oxopyrrolidin-1-yl)pyridin-2- yl)-1-isopropyl-7,7-dimethyl-N- (4-(methylcarbamoyl)benzyl)- 4,5,6,7-tetrahydro-1H- pyrazolo[4,3-c]pyridine-3- carboxamide |
| VE262 | 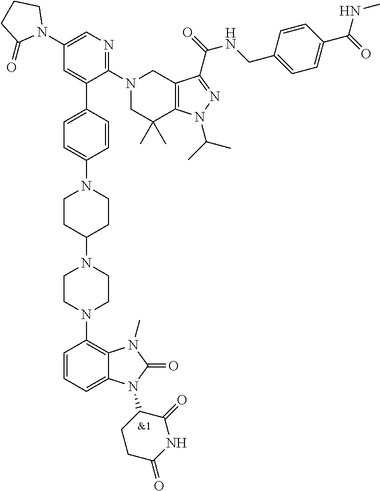 | rac-(R)-5-(3-(4-(4-(4-(1-(2,6- dioxopiperidin-3-yl)-3-methyl-2- oxo-2,3-dihydro-1H- benzo[d]imidazol-4-yl)piperazin- 1-yl)piperidin-1-yl)phenyl)-5-(2- oxopyrrolidin-1-yl)pyridin-2-yl)-1- isopropyl-7,7-dimethyl-N-(4- (methylcarbamoyl)benzyl)- 4,5,6,7-tetrahydro-1H- pyrazolo[4,3-c]pyridine-3- carboxamide |
| VE263 | 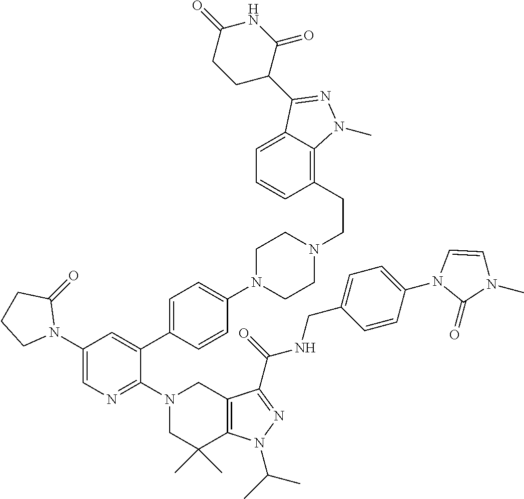 [SMILES: CC(C)n1nc(C(=O)NCc2ccc(-n3ccn(C)c3=O)cc2)c2c1C(C)(C)CN(c1ncc(N3CCCC3=O)cc1-c1ccc(N3CCN(CCc4cccc5c(C6CCC(=O)NC6=O)nn(C)c45)CC3)cc1)C2] | 5-(3-(4-(4-(2-(3-(2,6- dioxopiperidin-3-yl)-1-methyl-1H- indazol-7-yl)ethyl)piperazin-1- yl)phenyl)-5-(2-oxopyrrolidin-1- yl)pyridin-2-yl)-1-isopropyl-7,7- dimethyl-N-(4-(3-methyl-2-oxo- 2,3-dihydro-1H-imidazol-1- yl)benzyl)-4,5,6,7-tetrahydro-1H- pyrazolo[4,3-c]pyridine-3- carboxamide |
| VE264 | 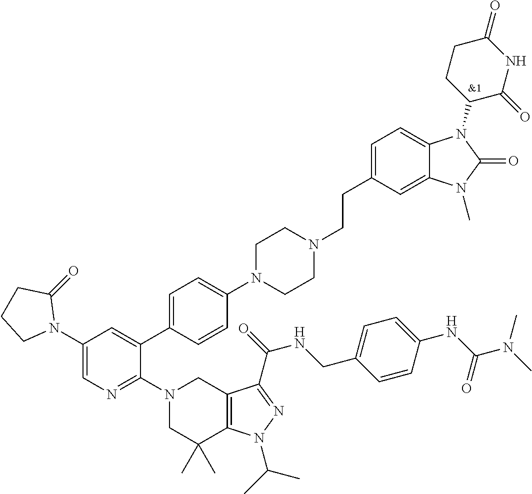 | rac-(R)-N-(4-(3,3- dimethylureido)benzyl)-5-(3-(4- (4-(2-(1-(2,6-dioxopiperidin-3-yl)- 3-methyl-2-oxo-2,3-dihydro-1H- benzo[d]imidazol-5- yl)ethyl)piperazin-1-yl)phenyl)-5- (2-oxopyrrolidin-1-yl)pyridin-2- yl)-1-isopropyl-7,7-dimethyl- 4,5,6,7-tetrahydro-1H- pyrazolo[4,3-c]pyridine-3- carboxamide |
| VE265 | 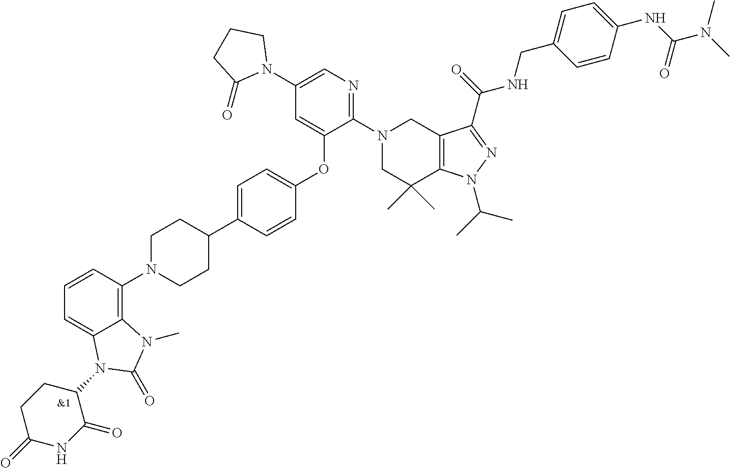 | rac-(S)-N-(4-(3,3- dimethylureido)benzyl)-5-(3-(4- (1-(1-(2,6-dioxopiperidin-3-yl)-3- methyl-2-oxo-2,3-dihydro-1H- benzo[d]imidazol-4-yl)piperidin- 4-yl)phenoxy)-5-(2-oxopyrrolidin- 1-yl)pyridin-2-yl)-1-isopropyl-7,7- dimethyl-4,5,6,7-tetrahydro-1H- pyrazolo[4,3-c]pyridine-3- carboxamide |
| VE266 | 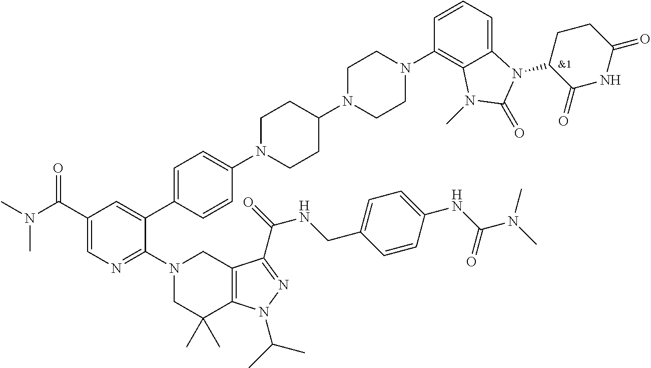 | rac-(R)-5-(5- (dimethylcarbamoyl)-3-(4-(4-(4- (1-(2,6-dioxopiperidin-3-yl)-3- methyl-2-oxo-2,3-dihydro-1H- benzo[d]imidazol-4-yl)piperazin- 1-yl)piperidin-1-yl)phenyl)pyridin- 2-yl)-N-(4-(3,3- dimethylureido)benzyl)-1- isopropyl-7,7-dimethyl-4,5,6,7- tetrahydro-1H-pyrazolo[4,3- c]pyridine-3-carboxamide |
| VE267 | 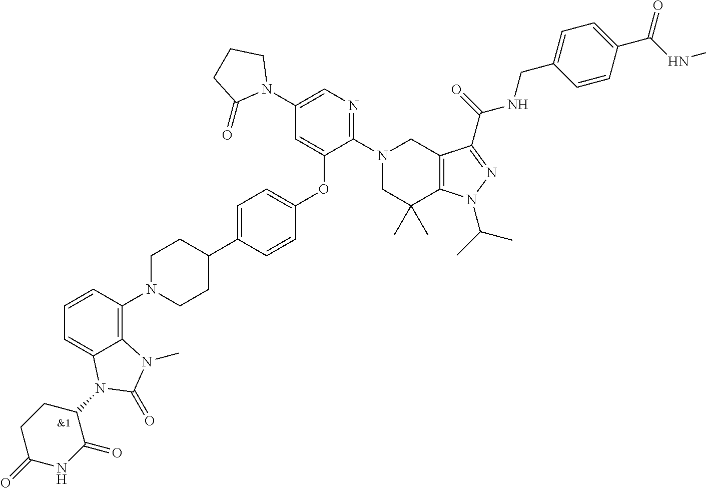 | rac-(S)-5-(3-(4-(1-(1-(2,6- dioxopiperidin-3-yl)-3-methyl-2- oxo-2,3-dihydro-1H- benzo[d]imidazol-4-yl)piperidin- 4-yl)phenoxy)-5-(2-oxopyrrolidin- 1-yl)pyridin-2-yl)-1-isopropyl-7,7- dimethyl-N-(4- (methylcarbamoyl)benzyl)- 4,5,6,7-tetrahydro-1H- pyrazolo[4,3-c]pyridine-3- carboxamide |
| VE268 | 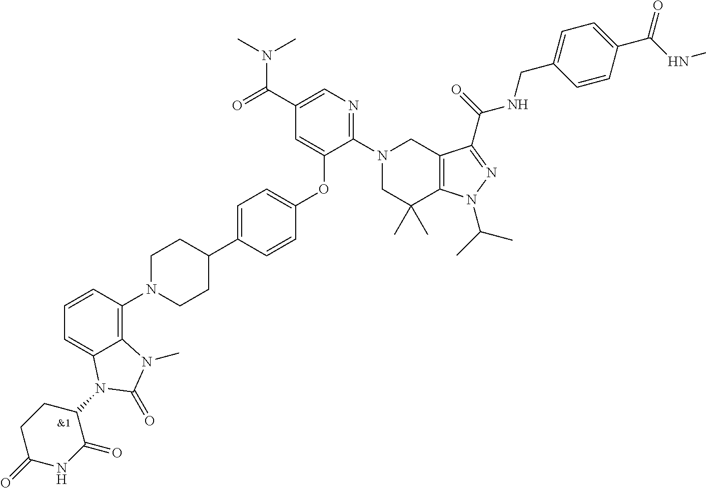 | rac-(S)-5-(5- (dimethylcarbamoyl)-3-(4-(1-(1- (2,6-dioxopiperidin-3-yl)-3- methyl-2-oxo-2,3-dihydro-1H- benzo[d]imidazol-4-yl)piperidin- 4-yl)phenoxy)pyridin-2-yl)-1- isopropyl-7,7-dimethyl-N-(4- (methylcarbamoyl)benzyl)- 4,5,6,7-tetrahydro-1H- pyrazolo[4,3-c]pyridine-3- carboxamide |
| VE269 | 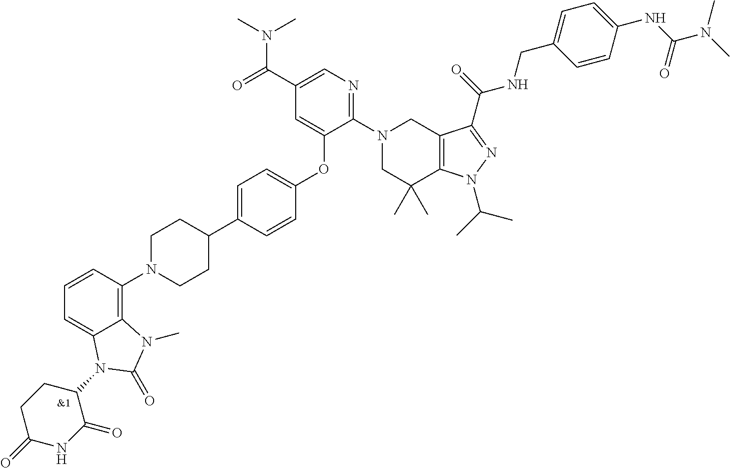 | rac-(S)-5-(5- (dimethylcarbamoyl)-3-(4-(1-(1- (2,6-dioxopiperidin-3-yl)-3- methyl-2-oxo-2,3-dihydro-1H- benzo[d]imidazol-4-yl)piperidin- 4-yl)phenoxy)pyridin-2-yl)-N-(4- (3,3-dimethylureido)benzyl)-1- isopropyl-7,7-dimethyl-4,5,6,7- tetrahydro-1H-pyrazolo[4,3- c]pyridine-3-carboxamide |
| VE270 | 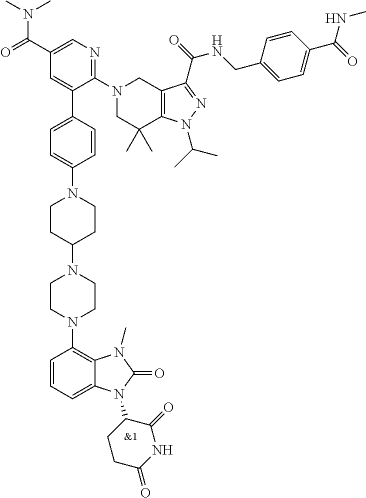 | rac-(R)-5-(5- (dimethylcarbamoyl)-3-(4-(4-(4- (1-(2,6-dioxopiperidin-3-yl)-3- methyl-2-oxo-2,3-dihydro-1H- benzo[d]imidazol-4-yl)piperazin- 1-yl)piperidin-1-yl)phenyl)pyridin- 2-yl)-1-isopropyl-7,7-dimethyl-N- (4-(methylcarbamoyl)benzyl)- 4,5,6,7-tetrahydro-1H- pyrazolo[4,3-c]pyridine-3- carboxamide |
| VE271 | 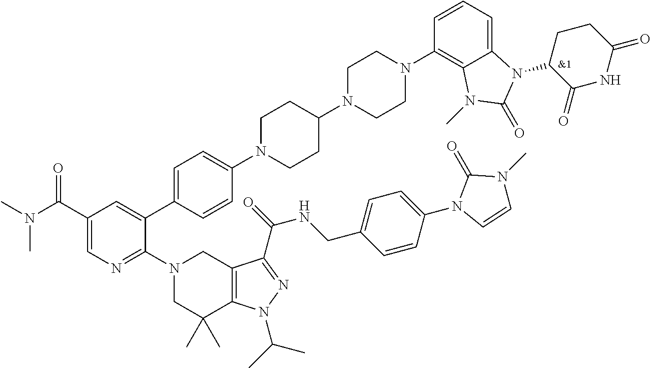 | rac-(R)-5-(5- (dimethylcarbamoyl)-3-(4-(4-(4- (1-(2,6-dioxopiperidin-3-yl)-3- methyl-2-oxo-2,3-dihydro-1H- benzo[d]imidazol-4-yl)piperazin- 1-yl)piperidin-1-yl)phenyl)pyridin- 2-yl)-1-isopropyl-7,7-dimethyl-N- (4-(3-methyl-2-oxo-2,3-dihydro- 1H-imidazol-1-yl)benzyl)-4,5,6,7- tetrahydro-1H-pyrazolo[4,3- c]pyridine-3-carboxamide |
| VE272 | 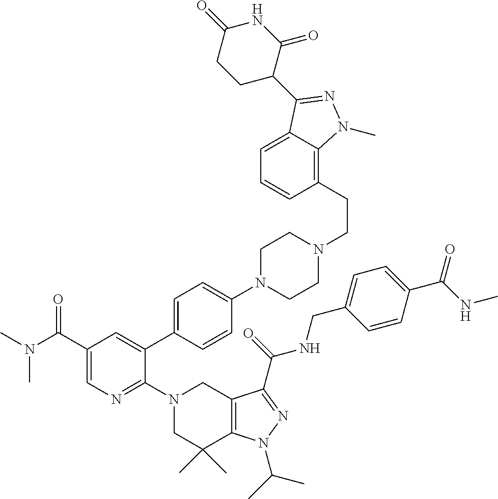 | 5-(5-(dimethylcarbamoyl)-3-(4- (4-(2-(3-(2,6-dioxopiperidin-3-yl)- 1-methyl-1H-indazol-7- yl)ethyl)piperazin-1- yl)phenyl)pyridin-2-yl)-1- isopropyl-7,7-dimethyl-N-(4- (methylcarbamoyl)benzyl)- 4,5,6,7-tetrahydro-1H- pyrazolo[4,3-c]pyridine-3- carboxamide |
| VE273 | 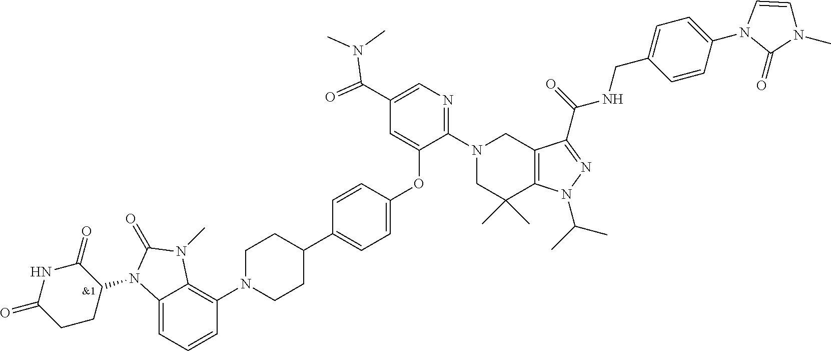 | rac-(R)-5-(5- (dimethylcarbamoyl)-3-(4-(1-(1- (2,6-dioxopiperidin-3-yl)-3- methyl-2-oxo-2,3-dihydro-1H- benzo[d]imidazol-4-yl)piperidin- 4-yl)phenoxy)pyridin-2-yl)-1- isopropyl-7,7-dimethyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE274 | 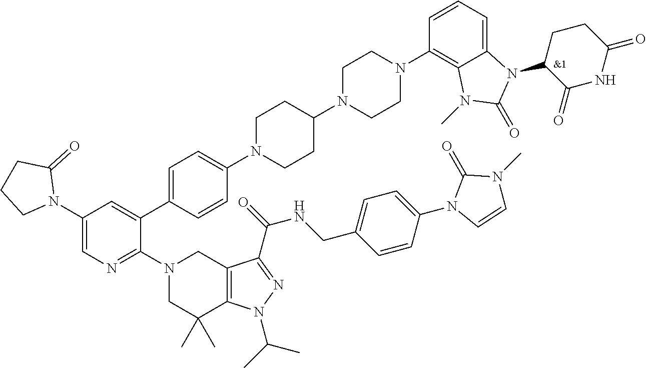 | rac-(R)-5-(3-(4-(4-(4-(1-(2,6- dioxopiperidin-3-yl)-3-methyl-2- oxo-2,3-dihydro-1H- benzo[d]imidazol-4-yl)piperazin- 1-yl)piperidin-1-yl)phenyl)-5-(2- oxopyrrolidin-1-yl)pyridin-2-yl)-1- isopropyl-7,7-dimethyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE275 | 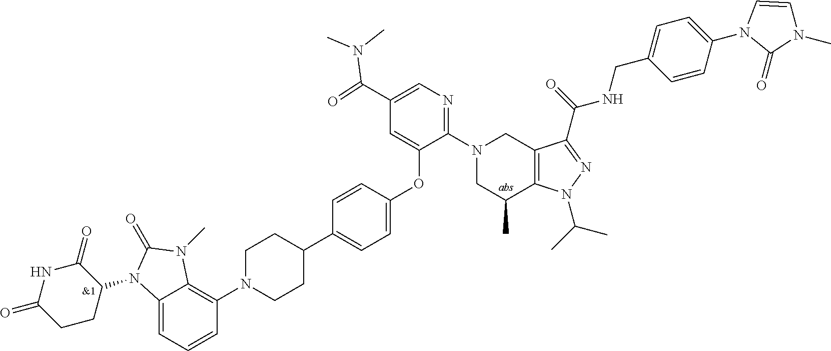 | (S)-5-(5-(dimethylcarbamoyl)-3- (4-(1-(1-((RS)-2,6-dioxopiperidin- 3-yl)-3-methyl-2-oxo-2,3-dihydro- 1H-benzo[d]imidazol-4- yl)piperidin-4-yl)phenoxy)pyridin- 2-yl)-1-isopropyl-7-methyl-N-(4- (3-methyl-2-oxo-2,3-dihydro-1H- imidazol-1-yl)benzyl)-4,5,6,7- tetrahydro-1H-pyrazolo[4,3- c]pyridine-3-carboxamide |
| VE276 | 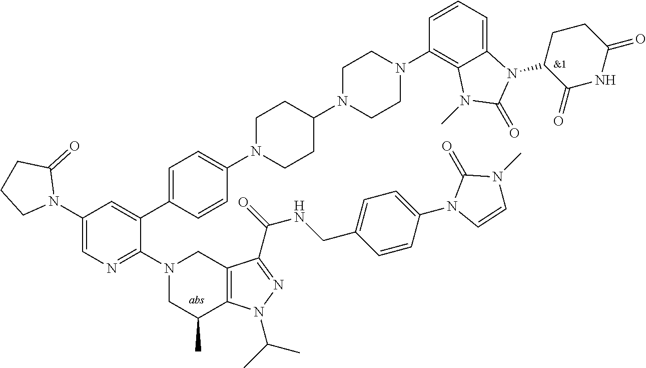 | (S)-5-(3-(4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)-5-(2- oxopyrrolidin-1-yl)pyridin-2-yl)-1- isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE277 | 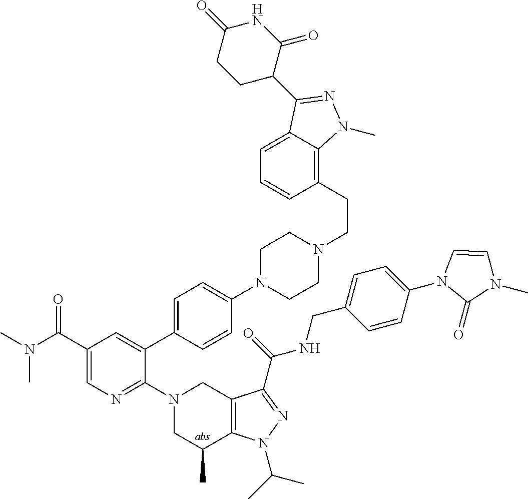 | (7S)-5-(5-(dimethylcarbamoyl)-3- (4-(4-(2-(3-(2,6-dioxopiperidin-3- yl)-1-methyl-1H-indazol-7- yl)ethyl)piperazin-1- yl)phenyl)pyridin-2-yl)-1- isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE278 | 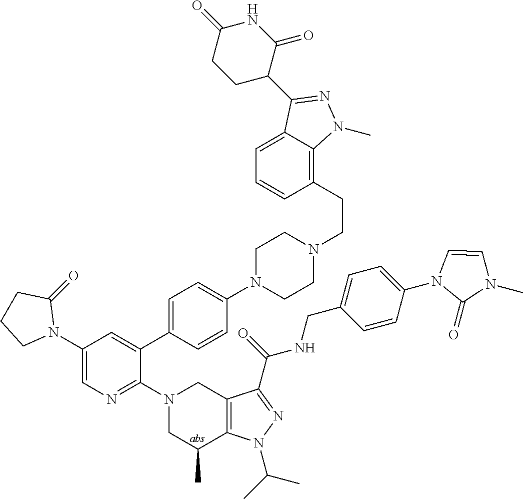 [SMILES: CC(C)n1nc(C(=O)NCc2ccc(-n3ccn(C)c3=O)cc2)c2c1[C@@H](C)CN(c1ncc(N3CCCC3=O)cc1-c1ccc(N3CCN(CCc4cccc5c(C6CCC(=O)NC6=O)nn(C)c45)CC3)cc1)C2] | (7S)-5-(3-(4-(4-(2-(3-(2,6- dioxopiperidin-3-yl)-1-methyl-1H- indazol-7-yl)ethyl)piperazin-1- yl)phenyl)-5-(2-oxopyrrolidin-1- yl)pyridin-2-yl)-1-isopropyl-7- methyl-N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE279 | 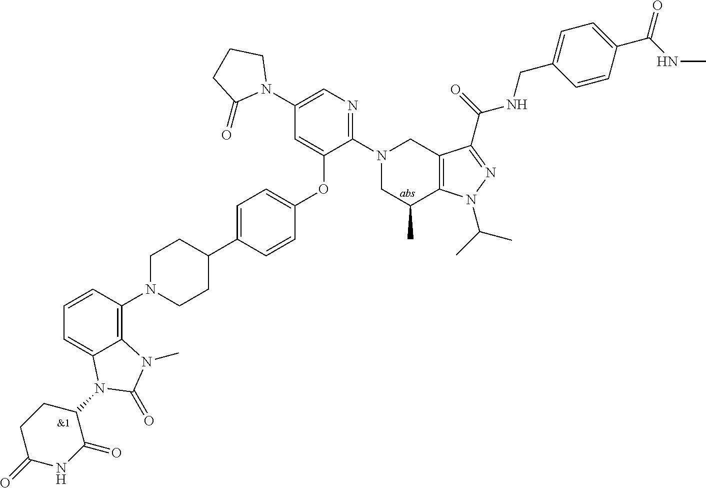 | (S)-5-(3-(4-(1-(1-((SR)-2,6- dioxopiperidin-3-yl)-3-methyl-2- oxo-2,3-dihydro-1H- benzo[d]imidazol-4-yl)piperidin- 4-yl)phenoxy)-5-(2-oxopyrrolidin- 1-yl)pyridin-2-yl)-1-isopropyl-7- methyl-N-(4- (methylcarbamoyl)benzyl)- 4,5,6,7-tetrahydro-1H- pyrazolo[4,3-c]pyridine-3- carboxamide |
| VE280 | 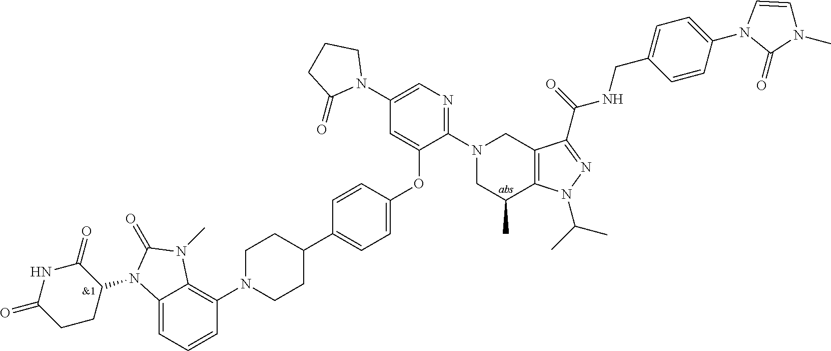 | (S)-5-(3-(4-(1-(1-((RS)-2,6- dioxopiperidin-3-yl)-3-methyl-2- oxo-2,3-dihydro-1H- benzo[d]imidazol-4-yl)piperidin- 4-yl)phenoxy)-5-(2-oxopyrrolidin- 1-yl)pyridin-2-yl)-1-isopropyl-7- methyl-N-(4-(3-methyl-2-oxo-2,3- dihydro-1H-imidazol-1- yl)benzyl)-4,5,6,7-tetrahydro-1H- pyrazolo[4,3-c]pyridine-3- carboxamide |
| VE281 | 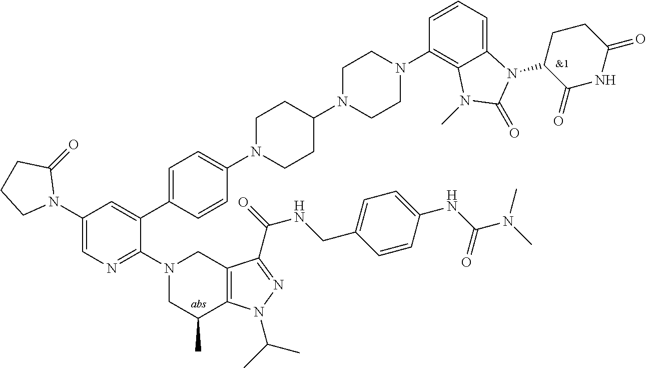 | (S)-N-(4-(3,3- dimethylureido)benzyl)-5-(3-(4- (4-(4-(1-((RS)-2,6-dioxopiperidin- 3-yl)-3-methyl-2-oxo-2,3-dihydro- 1H-benzo[d]imidazol-4- yl)piperazin-1-yl)piperidin-1- yl)phenyl)-5-(2-oxopyrrolidin-1- yl)pyridin-2-yl)-1-isopropyl-7- methyl-4,5,6,7-tetrahydro-1H- pyrazolo[4,3-c]pyridine-3- carboxamide |
| VE282 | 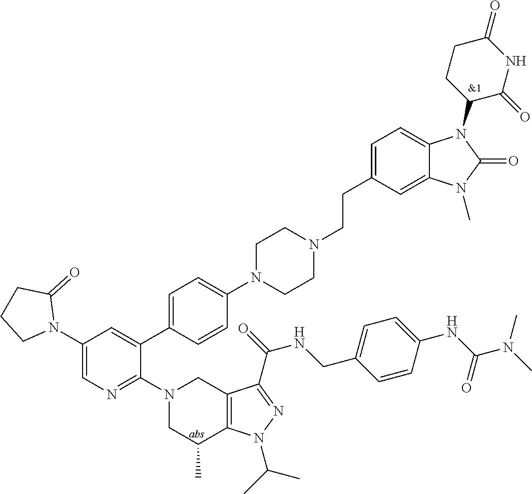 | (S)-N-(4-(3,3- dimethylureido)benzyl)-5-(3-(4- (4-(2-(1-((RS)-2,6-dioxopiperidin- 3-yl)-3-methyl-2-oxo-2,3-dihydro- 1H-benzo[d]imidazol-5- yl)ethyl)piperazin-1-yl)phenyl)-5- (2-oxopyrrolidin-1-yl)pyridin-2- yl)-1-isopropyl-7-methyl-4,5,6,7- tetrahydro-1H-pyrazolo[4,3- c]pyridine-3-carboxamide |
| VE283 | 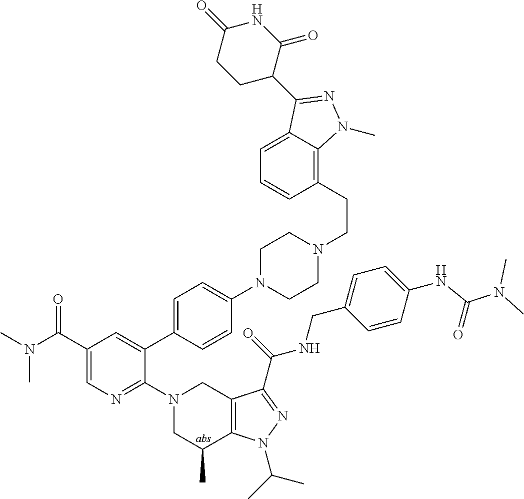 | (7S)-5-(5-(dimethylcarbamoyl)-3- (4-(4-(2-(3-(2,6-dioxopiperidin-3- yl)-1-methyl-1H-indazol-7- yl)ethyl)piperazin-1- yl)phenyl)pyridin-2-yl)-N-(4-(3,3- dimethylureido)benzyl)-1- isopropyl-7-methyl-4,5,6,7- tetrahydro-1H-pyrazolo[4,3- c]pyridine-3-carboxamide |
| VE284 | 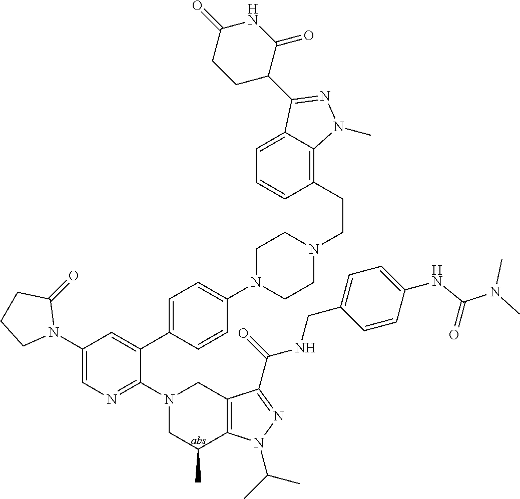 [SMILES: CC(C)n1nc(C(=O)NCc2ccc(NC(=O)N(C)C)cc2)c2c1[C@@H](C)CN(c1ncc(N3CCCC3=O)cc1-c1ccc(N3CCN(CCc4cccc5c(C6CCC(=O)NC6=O)nn(C)c45)CC3)cc1)C2] | (7S)-N-(4-(3,3- dimethylureido)benzyl)-5-(3-(4- (4-(2-(3-(2,6-dioxopiperidin-3-yl)- 1-methyl-1H-indazol-7- yl)ethyl)piperazin-1-yl)phenyl)-5- (2-oxopyrrolidin-1-yl)pyridin-2- yl)-1-isopropyl-7-methyl-4,5,6,7- tetrahydro-1H-pyrazolo[4,3- c]pyridine-3-carboxamide |
| VE285 | 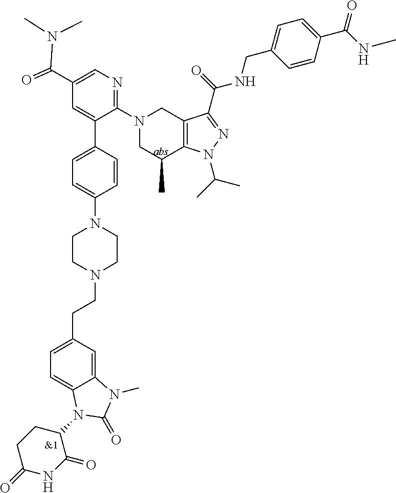 | (S)-5-(5-(dimethylcarbamoyl)-3- (4-(4-(2-(1-((RS)-2,6- dioxopiperidin-3-yl)-3-methyl-2- oxo-2,3-dihydro-1H- benzo[d]imidazol-5- yl)ethyl)piperazin-1- yl)phenyl)pyridin-2-yl)-1- isopropyl-7-methyl-N-(4- (methylcarbamoyl)benzyl)- 4,5,6,7-tetrahydro-1H- pyrazolo[4,3-c]pyridine-3- carboxamide |
| VE286 | 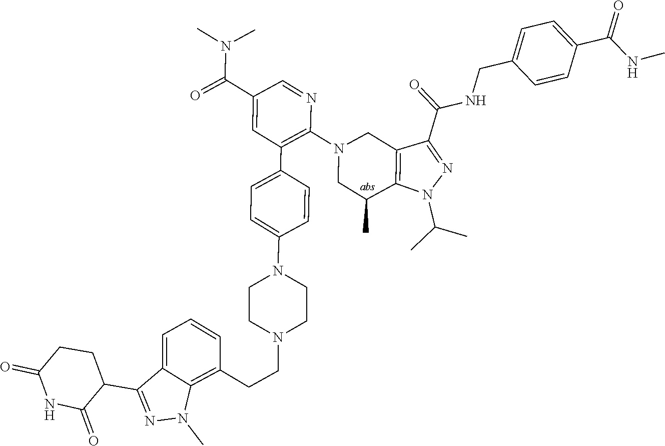 [SMILES: CNC(=O)c1ccc(CNC(=O)c2nn(C(C)C)c3c2CN(c2ncc(C(=O)N(C)C)cc2-c2ccc(N4CCN(CCc5cccc6c(C7CCC(=O)NC7=O)nn(C)c56)CC4)cc2)C[C@@H]3C)cc1] | (7S)-5-(5-(dimethylcarbamoyl)-3- (4-(4-(2-(3-(2,6-dioxopiperidin-3- yl)-1-methyl-1H-indazol-7- yl)ethyl)piperazin-1- yl)phenyl)pyridin-2-yl)-1- isopropyl-7-methyl-N-(4- (methylcarbamoyl)benzyl)- 4,5,6,7-tetrahydro-1H- pyrazolo[4,3-c]pyridine-3- carboxamide |
| VE287 | 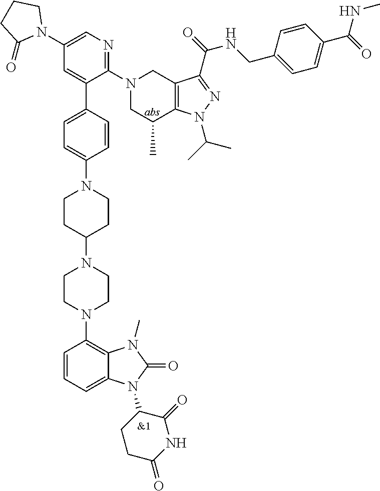 | (S)-5-(3-(4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)-5-(2- oxopyrrolidin-1-yl)pyridin-2-yl)-1- isopropyl-7-methyl-N-(4- (methylcarbamoyl)benzyl)- 4,5,6,7-tetrahydro-1H- pyrazolo[4,3-c]pyridine-3- carboxamide |
| VE290 | 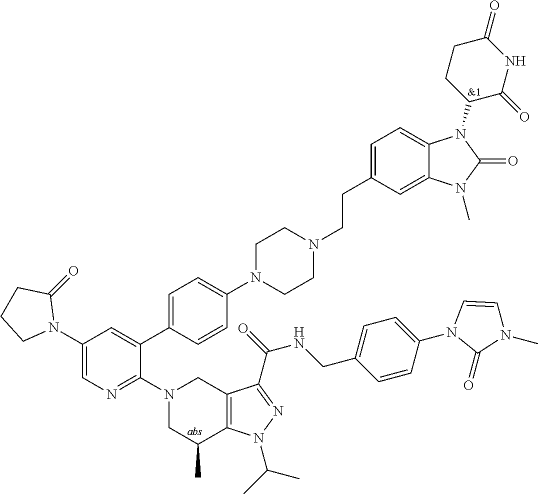 | (S)-5-(3-(4-(4-(2-(1-((RS)-2,6- dioxopiperidin-3-yl)-3-methyl-2- oxo-2,3-dihydro-1H- benzo[d]imidazol-5- yl)ethyl)piperazin-1-yl)phenyl)-5- (2-oxopyrrolidin-1-yl)pyridin-2- yl)-1-isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE291 | 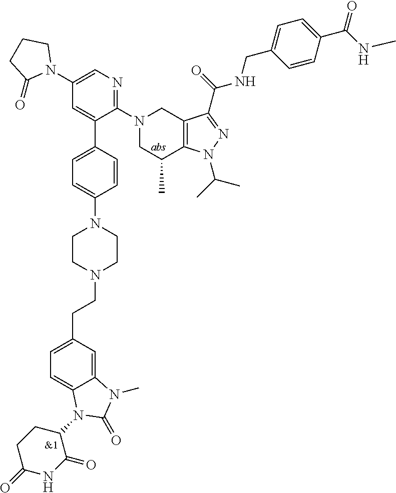 | (S)-5-(3-(4-(4-(2-(1-((RS)-2,6- dioxopiperidin-3-yl)-3-methyl-2- oxo-2,3-dihydro-1H- benzo[d]imidazol-5- yl)ethyl)piperazin-1-yl)phenyl)-5- (2-oxopyrrolidin-1-yl)pyridin-2- yl)-1-isopropyl-7-methyl-N-(4- (methylcarbamoyl)benzyl)- 4,5,6,7-tetrahydro-1H- pyrazolo[4,3-c]pyridine-3- carboxamide |
| VE292 | 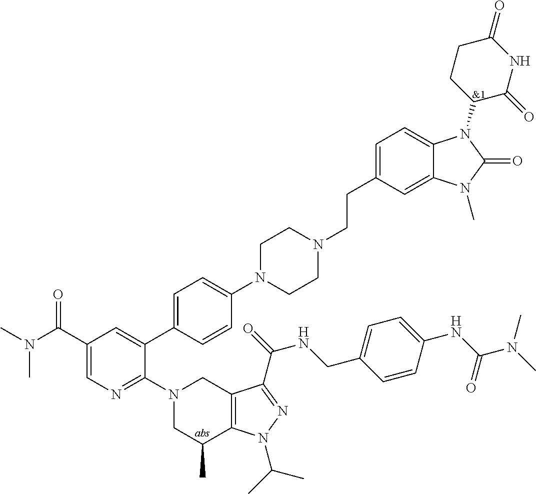 | (S)-5-(5-(dimethylcarbamoyl)-3- (4-(4-(2-(1-((RS)-2,6- dioxopiperidin-3-yl)-3-methyl-2- oxo-2,3-dihydro-1H- benzo[d]imidazol-5- yl)ethyl)piperazin-1- yl)phenyl)pyridin-2-yl)-N-(4-(3,3- dimethylureido)benzyl)-1- isopropyl-7-methyl-4,5,6,7- tetrahydro-1H-pyrazolo[4,3- c]pyridine-3-carboxamide |
| VE293 | 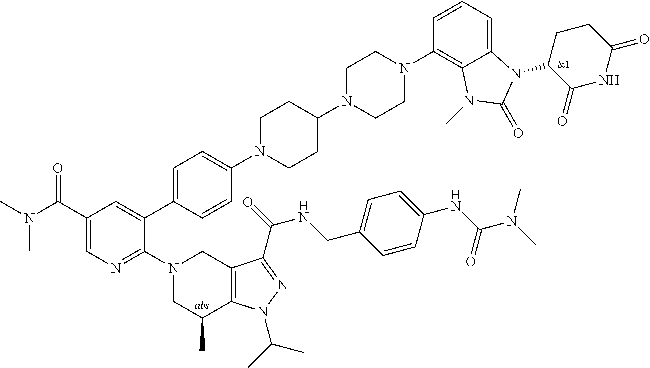 | (S)-5-(5-(dimethylcarbamoyl)-3- (4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)pyridin- 2-yl)-N-(4-(3,3- dimethylureido)benzyl)-1- isopropyl-7-methyl-4,5,6,7- tetrahydro-1H-pyrazolo[4,3- c]pyridine-3-carboxamide |
| VE294 | 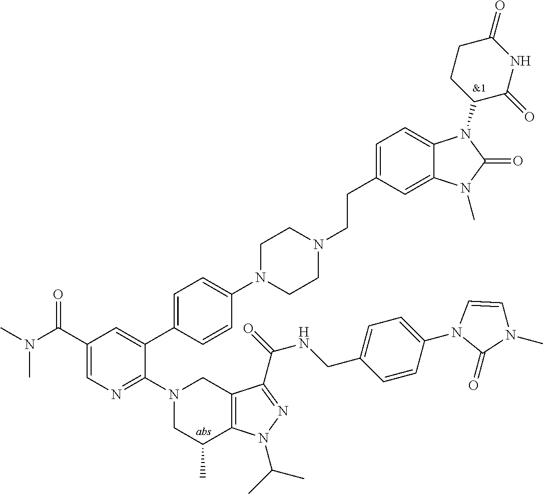 | (S)-5-(5-(dimethylcarbamoyl)-3- (4-(4-(2-(1-((RS)-2,6- dioxopiperidin-3-yl)-3-methyl-2- oxo-2,3-dihydro-1H- benzo[d]imidazol-5- yl)ethyl)piperazin-1- yl)phenyl)pyridin-2-yl)-1- isopropyl-7-methyl-N-(4-(3- methyl-2-oxo-2,3-dihydro-1H- imidazol-1-yl)benzyl)-4,5,6,7- tetrahydro-1H-pyrazolo[4,3- c]pyridine-3-carboxamide |
| VE295 | 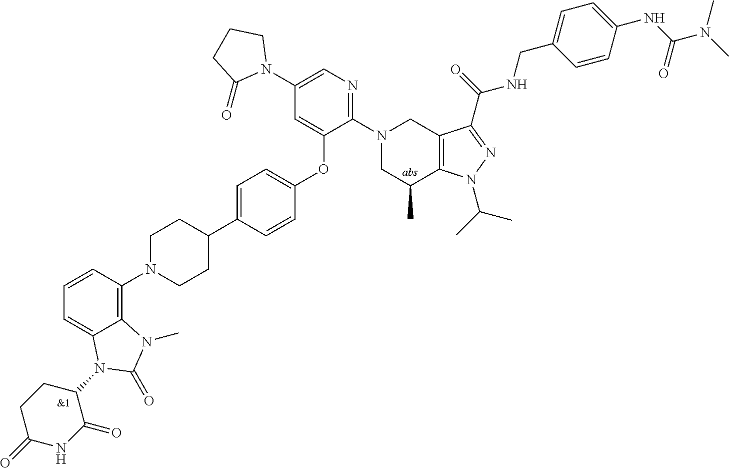 | (S)-N-(4-(3,3- dimethylureido)benzyl)-5-(3-(4- (1-(1-((SR)-2,6-dioxopiperidin-3- yl)-3-methyl-2-oxo-2,3-dihydro- 1H-benzo[d]imidazol-4- yl)piperidin-4-yl)phenoxy)-5-(2- oxopyrrolidin-1-yl)pyridin-2-yl)-1- isopropyl-7-methyl-4,5,6,7- tetrahydro-1H-pyrazolo[4,3- c]pyridine-3-carboxamide |
| VE296 | 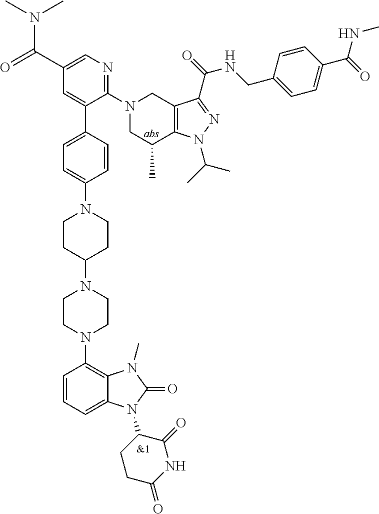 | (S)-5-(5-(dimethylcarbamoyl)-3- (4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)pyridin- 2-yl)-1-isopropyl-7-methyl-N-(4- (methylcarbamoyl)benzyl)- 4,5,6,7-tetrahydro-1H- pyrazolo[4,3-c]pyridine-3- carboxamide |
| VE297 | 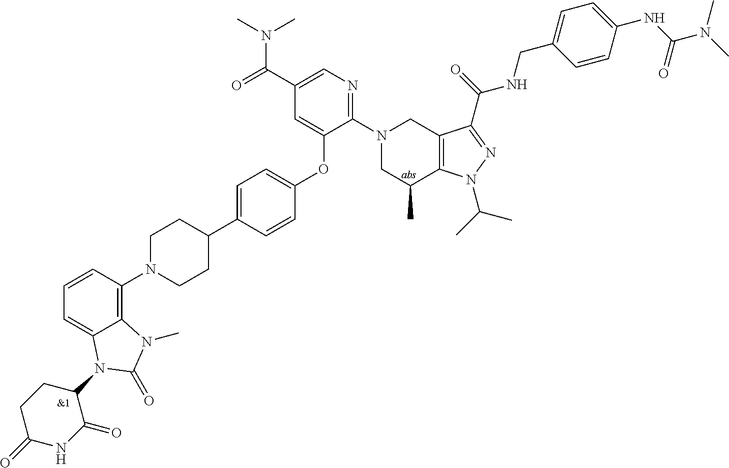 | (S)-5-(5-(dimethylcarbamoyl)-3- (4-(1-(1-((SR)-2,6-dioxopiperidin- 3-yl)-3-methyl-2-oxo-2,3-dihydro- 1H-benzo[d]imidazol-4- yl)piperidin-4-yl)phenoxy)pyridin- 2-yl)-N-(4-(3,3- dimethylureido)benzyl)-1- isopropyl-7-methyl-4,5,6,7- tetrahydro-1H-pyrazolo[4,3- c]pyridine-3-carboxamide |
| VE298 | 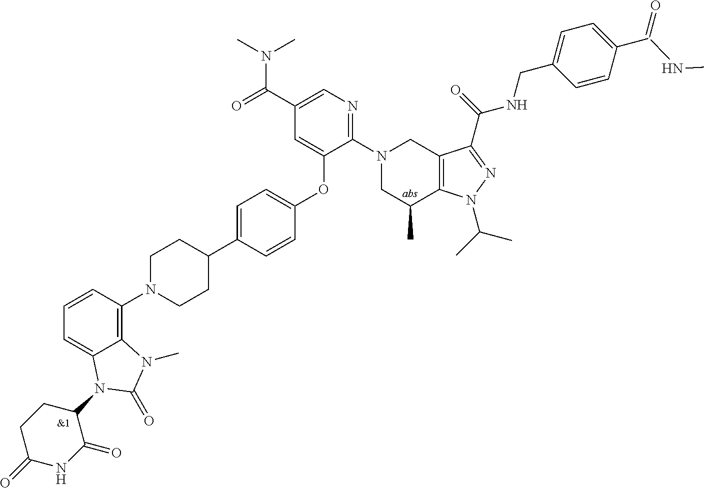 | (S)-5-(5-(dimethylcarbamoyl)-3- (4-(1-(1-((SR)-2,6-dioxopiperidin- 3-yl)-3-methyl-2-oxo-2,3-dihydro- 1H-benzo[d]imidazol-4- yl)piperidin-4-yl)phenoxy)pyridin- 2-yl)-1-isopropyl-7-methyl-N-(4- (methylcarbamoyl)benzyl)- 4,5,6,7-tetrahydro-1H- pyrazolo[4,3-c]pyridine-3- carboxamide |
| VE299 | 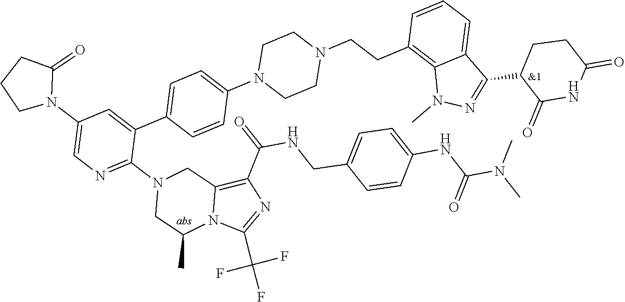 | (S)-N-(4-(3,3- dimethylureido)benzyl)-7-(3-(4- (4-(2-(3-((SR)-2,6-dioxopiperidin- 3-yl)-1-methyl-1H-indazol-7- yl)ethyl)piperazin-1-yl)phenyl)-5- (2-oxopyrrolidin-1-yl)pyridin-2- yl)-5-methyl-3-(trifluoromethyl)- 5,6,7,8-tetrahydroimidazo[1,5- a]pyrazine-1-carboxamide |
| VE300 | 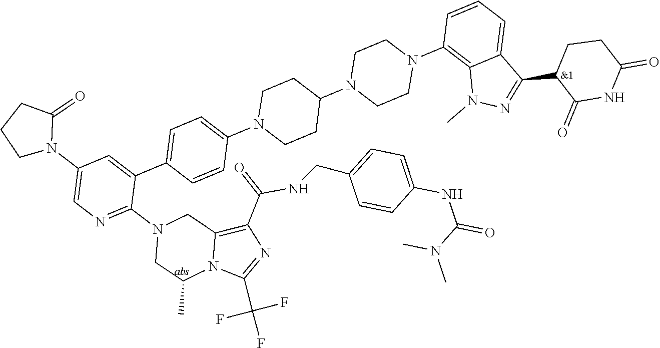 | (S)-N-(4-(3,3- dimethylureido)benzyl)-7-(3-(4- (4-(4-(1-((RS)-2,6-dioxopiperidin- 3-yl)-3-methyl-2-oxo-2,3-dihydro- 1H-benzo[d]imidazol-4- yl)piperazin-1-yl)piperidin-1- yl)phenyl)-5-(2-oxopyrrolidin-1- yl)pyridin-2-yl)-5-methyl-3- (trifluoromethyl)-5,6,7,8- tetrahydroimidazo[1,5- a]pyrazine-1-carboxamide |
| VE301 | 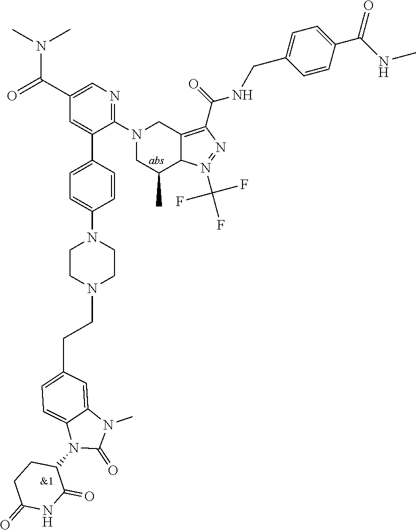 | (S)-7-(5-(dimethylcarbamoyl)-3- (4-(4-(2-(1-((RS)-2,6- dioxopiperidin-3-yl)-3-methyl-2- oxo-2,3-dihydro-1H- benzo[d]imidazol-5- yl)ethyl)piperazin-1- yl)phenyl)pyridin-2-yl)-5-methyl- N-(4-(methylcarbamoyl)benzyl)- 3-(trifluoromethyl)-5,6,7,8- tetrahydroimidazo[1,5- a]pyrazine-1-carboxamide |
| VE302 | 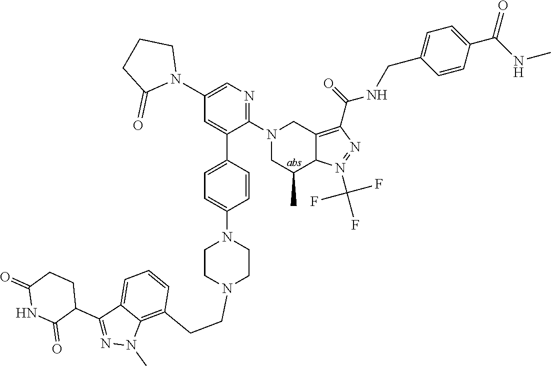 | (5S)-7-(3-(4-(4-(2-(3-(2,6- dioxopiperidin-3-yl)-1-methyl-1H- indazol-7-yl)ethyl)piperazin-1- yl)phenyl)-5-(2-oxopyrrolidin-1- yl)pyridin-2-yl)-5-methyl-N-(4- (methylcarbamoyl)benzyl)-3- (trifluoromethyl)-5,6,7,8- tetrahydroimidazo[1,5- a]pyrazine-1-carboxamide |
| VE303 | 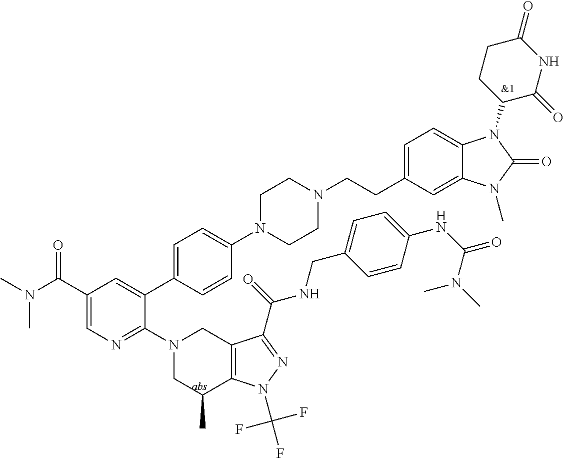 | (S)-7-(5-(dimethylcarbamoyl)-3- (4-(4-(2-(1-((RS)-2,6- dioxopiperidin-3-yl)-3-methyl-2- oxo-2,3-dihydro-1H- benzo[d]imidazol-5- yl)ethyl)piperazin-1- yl)phenyl)pyridin-2-yl)-N-(4-(3,3- dimethylureido)benzyl)-5-methyl- 3-(trifluoromethyl)-5,6,7,8- tetrahydroimidazo[1,5- a]pyrazine-1-carboxamide |
| VE304 | 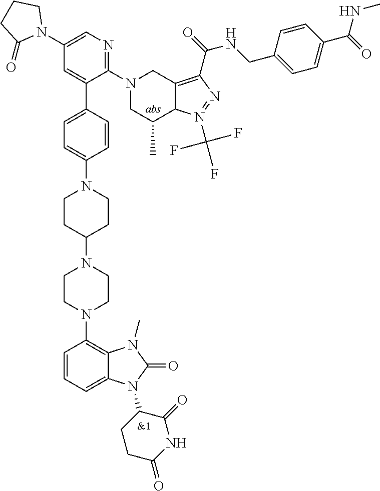 | (S)-7-(3-(4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)-5-(2- oxopyrrolidin-1-yl)pyridin-2-yl)-5- methyl-N-(4- (methylcarbamoyl)benzyl)-3- (trifluoromethyl)-5,6,7,8- tetrahydroimidazo[1,5- a]pyrazine-1-carboxamide |
| VE305 | 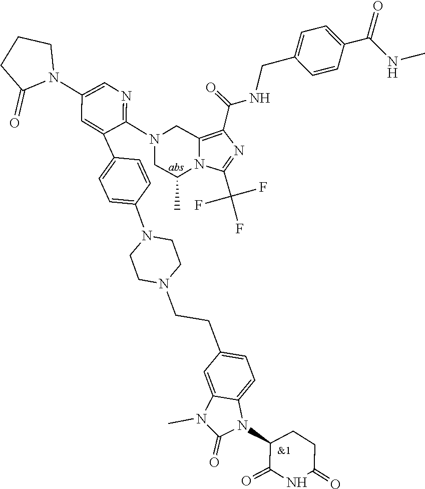 [SMILES: CNC(=O)c1ccc(CNC(=O)c2nc(C(F)(F)F)n3c2CN(c2ncc(N4CCCC4=O)cc2-c2ccc(N4CCN(CCc5ccc6c(c5)n(C)c(=O)n6[C@H]5CCC(=O)NC5=O)CC4)cc2)C[C@H]3C)cc1] | (S)-7-(3-(4-(4-(2-(1-((RS)-2,6- dioxopiperidin-3-yl)-3-methyl-2- oxo-2,3-dihydro-1H- benzo[d]imidazol-5- yl)ethyl)piperazin-1-yl)phenyl)-5- (2-oxopyrrolidin-1-yl)pyridin-2- yl)-5-methyl-N-(4- (methylcarbamoyl)benzyl)-3- (trifluoromethyl)-5,6,7,8- tetrahydroimidazo[1,5- a]pyrazine-1-carboxamide |
| VE306 | 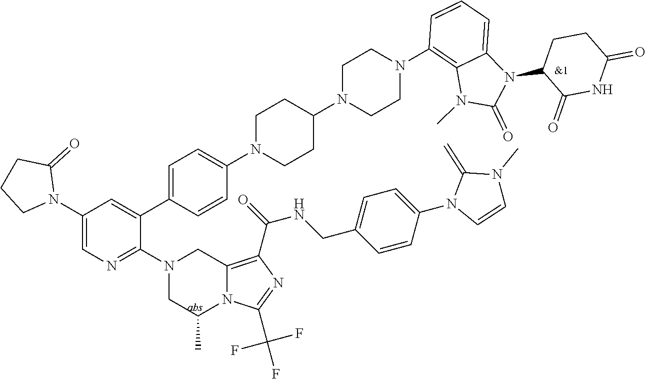 | (S)-7-(3-(4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)-5-(2- oxopyrrolidin-1-yl)pyridin-2-yl)-5- methyl-N-(4-(3-methyl-2-oxo-2,3- dihydro-1H-imidazol-1- yl)benzyl)-3-(trifluoromethyl)- 5,6,7,8-tetrahydroimidazo[1,5- a]pyrazine-1-carboxamide |
| VE307 | 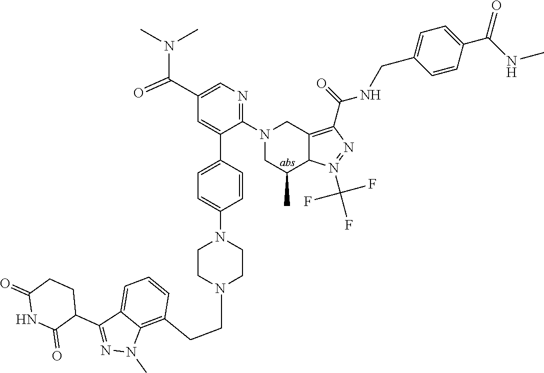 | (5S)-7-(5-(dimethylcarbamoyl)-3- (4-(4-(2-(3-(2,6-dioxopiperidin-3- yl)-1-methyl-1H-indazol-7- yl)ethyl)piperazin-1- yl)phenyl)pyridin-2-yl)-5-methyl- N-(4-(methylcarbamoyl)benzyl)- 3-(trifluoromethyl)-5,6,7,8- tetrahydroimidazo[1,5- a]pyrazine-1-carboxamide |
| VE308 | 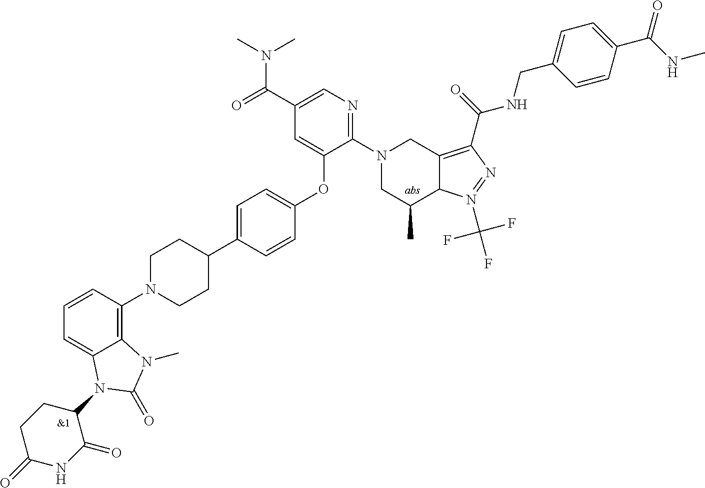 | (S)-7-(5-(dimethylcarbamoyl)-3- (4-(1-(1-((RS)-2,6-dioxopiperidin- 3-yl)-3-methyl-2-oxo-2,3-dihydro- 1H-benzo[d]imidazol-4- yl)piperidin-4-yl)phenoxy)pyridin- 2-yl)-5-methyl-N-(4- (methylcarbamoyl)benzyl)-3- (trifluoromethyl)-5,6,7,8- tetrahydroimidazo[1,5- a]pyrazine-1-carboxamide |
| VE309 | 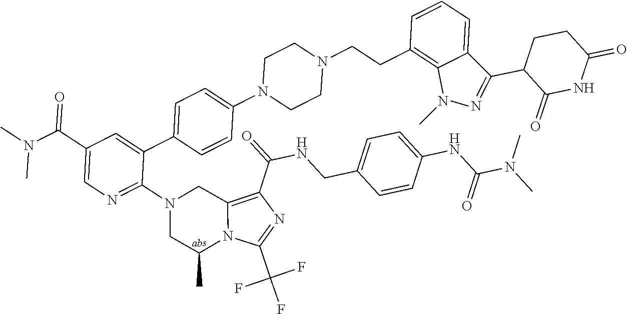 [SMILES: C[C@H]1CN(c2ncc(C(=O)N(C)C)cc2-c2ccc(N3CCN(CCc4cccc5c(C6CCC(=O)NC6=O)nn(C)c45)CC3)cc2)Cc2c(C(=O)NCc3ccc(NC(=O)N(C)C)cc3)nc(C(F)(F)F)n21] | (5S)-7-(5-(dimethylcarbamoyl)-3- (4-(4-(2-(3-(2,6-dioxopiperidin-3- yl)-1-methyl-1H-indazol-7- yl)ethyl)piperazin-1- yl)phenyl)pyridin-2-yl)-N-(4-(3,3- dimethylureido)benzyl)-5-methyl- 3-(trifluoromethyl)-5,6,7,8- tetrahydroimidazo[1,5- a]pyrazine-1-carboxamide |
| VE310 | 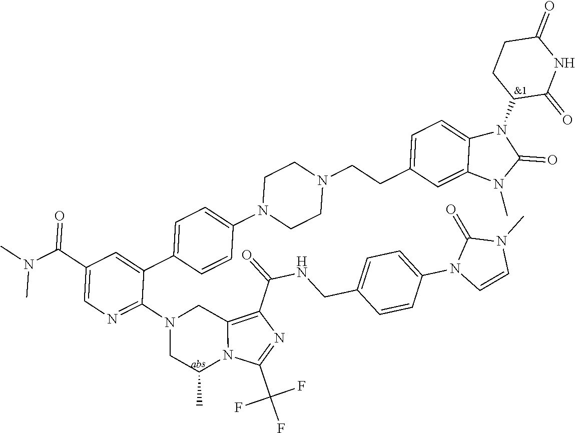 | (S)-7-(5-(dimethylcarbamoyl)-3- (4-(4-(2-(1-((RS)-2,6- dioxopiperidin-3-yl)-3-methyl-2- oxo-2,3-dihydro-1H- benzo[d]imidazol-5- yl)ethyl)piperazin-1- yl)phenyl)pyridin-2-yl)-5-methyl- N-(4-(3-methyl-2-oxo-2,3- dihydro-1H-imidazol-1- yl)benzyl)-3-(trifluoromethyl)- 5,6,7,8-tetrahydroimidazo[1,5- a]pyrazine-1-carboxamide |
| VE311 | 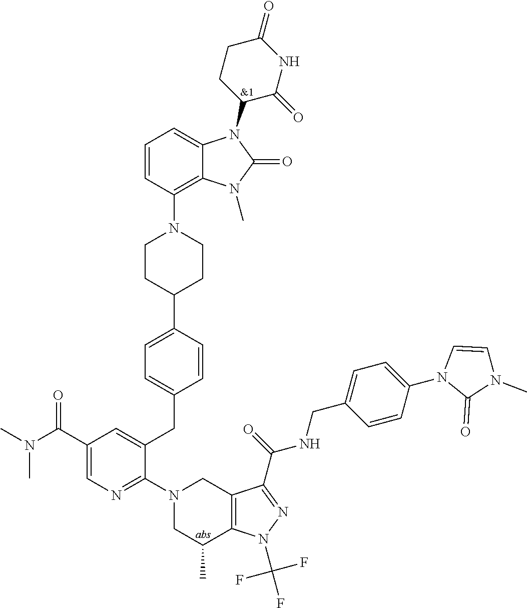 | (S)-7-(5-(dimethylcarbamoyl)-3- (4-(1-(1-((RS)-2,6-dioxopiperidin- 3-yl)-3-methyl-2-oxo-2,3-dihydro- 1H-benzo[d]imidazol-4- yl)piperidin-4-yl)phenoxy)pyridin- 2-yl)-5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE312 | 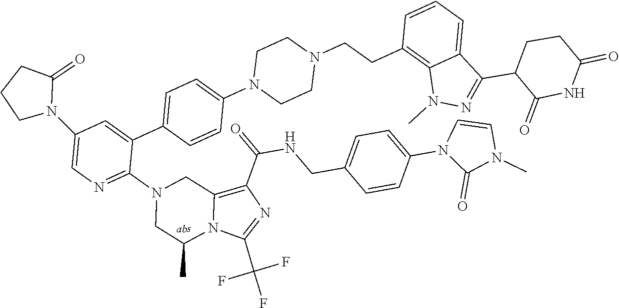 [SMILES: C[C@H]1CN(c2ncc(N3CCCC3=O)cc2-c2ccc(N3CCN(CCc4cccc5c(C6CCC(=O)NC6=O)nn(C)c45)CC3)cc2)Cc2c(C(=O)NCc3ccc(-n4ccn(C)c4=O)cc3)nc(C(F)(F)F)n21] | (5S)-7-(3-(4-(4-(2-(3-(2,6- dioxopiperidin-3-yl)-1-methyl-1H- indazol-7-yl)ethyl)piperazin-1- yl)phenyl)-5-(2-oxopyrrolidin-1- yl)pyridin-2-yl)-5-methyl-N-(4-(3- methyl-2-oxo-2,3-dihydro-1H- imidazol-1-yl)benzyl)-3- (trifluoromethyl)-5,6,7,8- tetrahydroimidazo[1,5- a]pyrazine-1-carboxamide |
| VE313 | 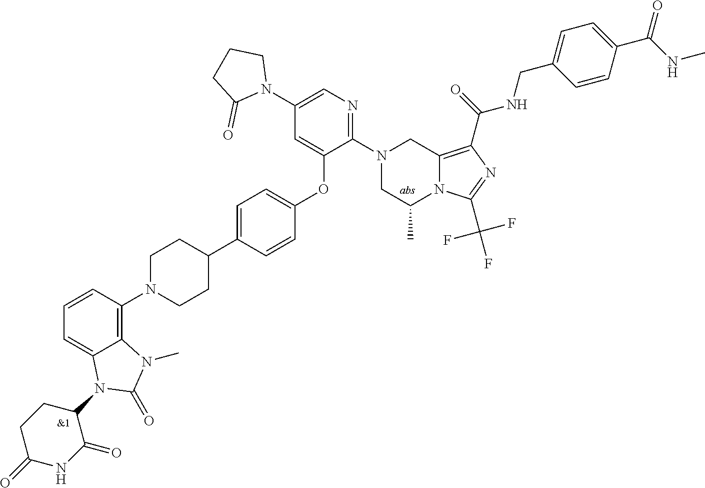 | (S)-7-(3-(4-(1-(1-((RS)-2,6- dioxopiperidin-3-yl)-3-methyl-2- oxo-2,3-dihydro-1H- benzo[d]imidazol-4-yl)piperidin- 4-yl)phenoxy)-5-(2-oxopyrrolidin- 1-yl)pyridin-2-yl)-5-methyl-N-(4- (methylcarbamoyl)benzyl)-3- (trifluoromethyl)-5,6,7,8- tetrahydroimidazo[1,5- a]pyrazine-1-carboxamide |
| VE314 | 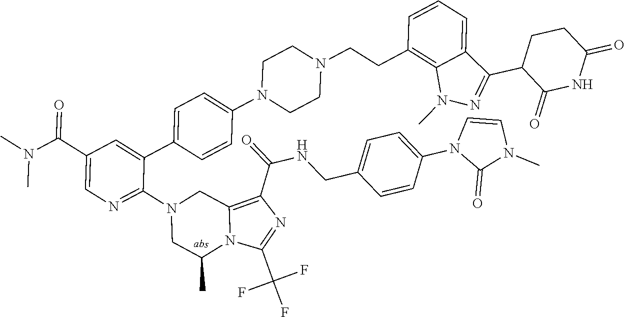 [SMILES: C[C@H]1CN(c2ncc(C(=O)N(C)C)cc2-c2ccc(N3CCN(CCc4cccc5c(C6CCC(=O)NC6=O)nn(C)c45)CC3)cc2)Cc2c(C(=O)NCc3ccc(-n4ccn(C)c4=O)cc3)nc(C(F)(F)F)n21] | (5S)-7-(5-(dimethylcarbamoyl)-3- (4-(4-(2-(3-(2,6-dioxopiperidin-3- yl)-1-methyl-1H-indazol-7- yl)ethyl)piperazin-1- yl)phenyl)pyridin-2-yl)-5-methyl- N-(4-(3-methyl-2-oxo-2,3- dihydro-1H-imidazol-1- yl)benzyl)-3-(trifluoromethyl)- 5,6,7,8-tetrahydroimidazo[1,5- a]pyrazine-1-carboxamide |
| VE315 | 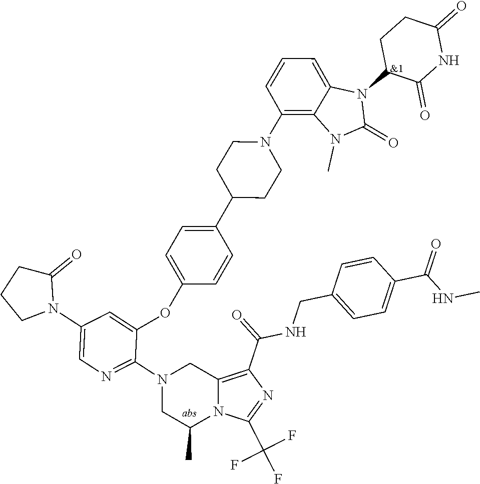 | (S)-N-(4-(3,3- dimethylureido)benzyl)-7-(3-(4- (1-(1-((RS)-2,6-dioxopiperidin-3- yl)-3-methyl-2-oxo-2,3-dihydro- 1H-benzo[d]imidazol-4- yl)piperidin-4-yl)phenoxy)-5-(2- oxopyrrolidin-1-yl)pyridin-2-yl)-5- methyl-3-(trifluoromethyl)- 5,6,7,8-tetrahydroimidazo[1,5- a]pyrazine-1-carboxamide |
| VE316 | 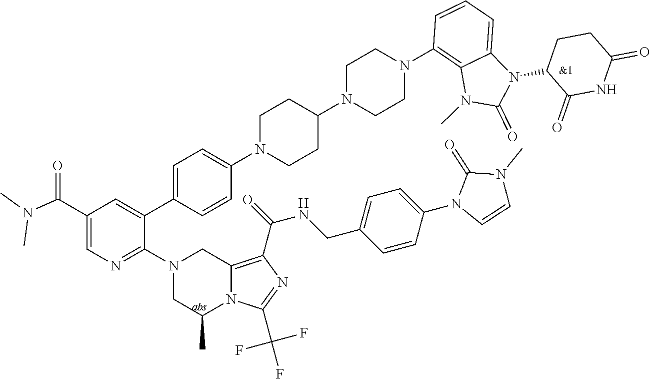 | (S)-7-(5-(dimethylcarbamoyl)-3- (4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)pyridin- 2-yl)-5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE317 | 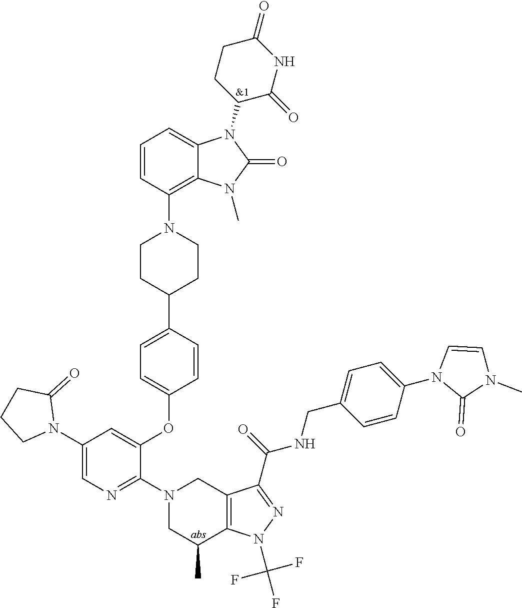 | (S)-7-(3-(4-(1-(1-((RS)-2,6- dioxopiperidin-3-yl)-3-methyl-2- oxo-2,3-dihydro-1H- benzo[d]imidazol-4-yl)piperidin- 4-yl)phenoxy)-5-(2-oxopyrrolidin- 1-yl)pyridin-2-yl)-5-methyl-N-(4- (3-methyl-2-oxo-2,3-dihydro-1H- imidazol-1-yl)benzyl)-3- (trifluoromethyl)-5,6,7,8- tetrahydroimidazo[1,5- a]pyrazine-1-carboxamide |
| VE318 | 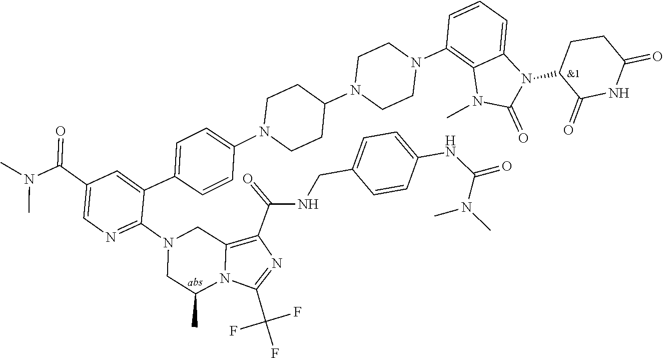 | (S)-7-(5-(dimethylcarbamoyl)-3- (4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)pyridin- 2-yl)-N-(4-(3,3- dimethylureido)benzyl)-5-methyl- 3-(trifluoromethyl)-5,6,7,8- tetrahydroimidazo[1,5- a]pyrazine-1-carboxamide |
| VE319 | 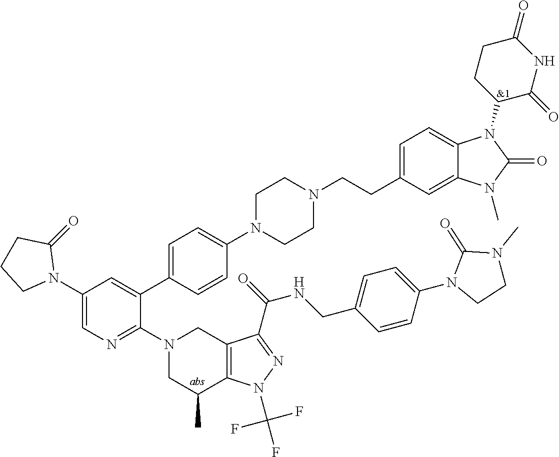 | (S)-7-(3-(4-(4-(2-(1-((RS)-2,6- dioxopiperidin-3-yl)-3-methyl-2- oxo-2,3-dihydro-1H- benzo[d]imidazol-5- yl)ethyl)piperazin-1-yl)phenyl)-5- (2-oxopyrrolidin-1-yl)pyridin-2- yl)-5-methyl-N-(4-(3-methyl-2- oxo-2,3-dihydro-1H-imidazol-1- yl)benzyl)-3-(trifluoromethyl)- 5,6,7,8-tetrahydroimidazo[1,5- a]pyrazine-1-carboxamide |
| VE320 | 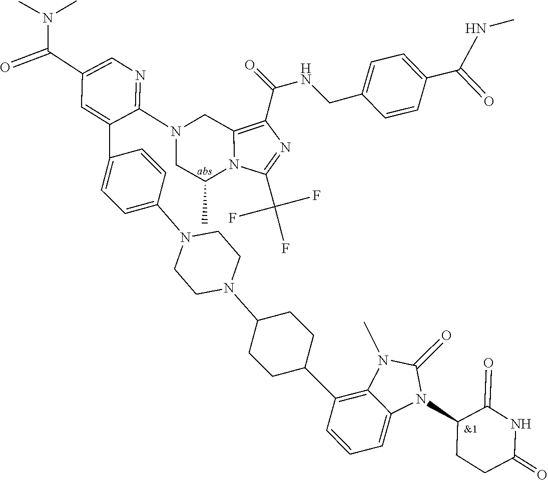 [SMILES: CNC(=O)c1ccc(CNC(=O)c2nc(C(F)(F)F)n3c2CN(c2ncc(C(=O)N(C)C)cc2-c2ccc(N4CCN(C5CCC(c6cccc7c6n(C)c(=O)n7[C@@H]6CCC(=O)NC6=O)CC5)CC4)cc2)C[C@H]3C)cc1] | (S)-7-(5-(dimethylcarbamoyl)-3- (4-(4-(4-(1-((RS)-2,6- dioxopiperidin-3-yl)-3-methyl-2- oxo-2,3-dihydro-1H- benzo[d]imidazol-4-yl)piperazin- 1-yl)piperidin-1-yl)phenyl)pyridin- 2-yl)-5-methyl-N-(4- (methylcarbamoyl)benzyl)-3- (trifluoromethyl)-5,6,7,8- tetrahydroimidazo[1,5- a]pyrazine-1-carboxamide |
| VE321 | 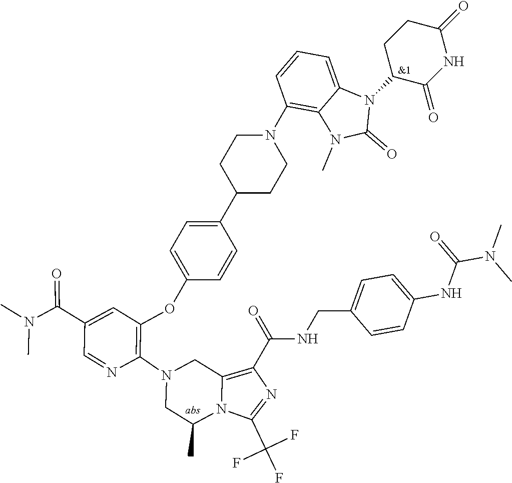 | (S)-7-(5-(dimethylcarbamoyl)-3- (4-(1-(1-((RS)-2,6-dioxopiperidin- 3-yl)-3-methyl-2-oxo-2,3-dihydro- 1H-benzo[d]imidazol-4- yl)piperidin-4-yl)phenoxy)pyridin- 2-yl)-N-(4-(3,3- dimethylureido)benzyl)-5-methyl- 3-(trifluoromethyl)-5,6,7,8- tetrahydroimidazo[1,5- a]pyrazine-1-carboxamide |
| VE322 | 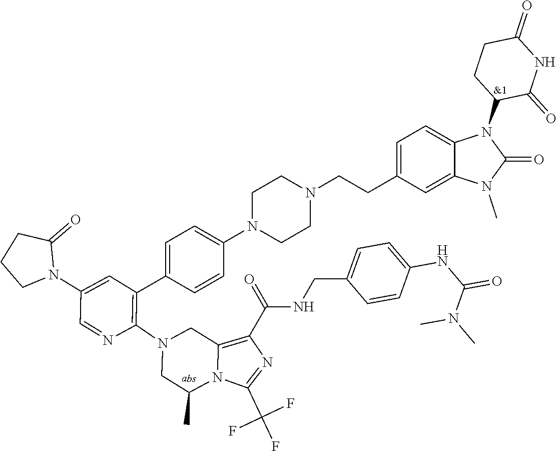 | (S)-N-(4-(3,3- dimethylureido)benzyl)-7-(3-(4- (4-(2-(1-((RS)-2,6-dioxopiperidin- 3-yl)-3-methyl-2-oxo-2,3-dihydro- 1H-benzo[d]imidazol-5- yl)ethyl)piperazin-1-yl)phenyl)-5- (2-oxopyrrolidin-1-yl)pyridin-2- yl)-5-methyl-3-(trifluoromethyl)- 5,6,7,8-tetrahydroimidazo[1,5- a]pyrazine-1-carboxamide |
| VE323 | 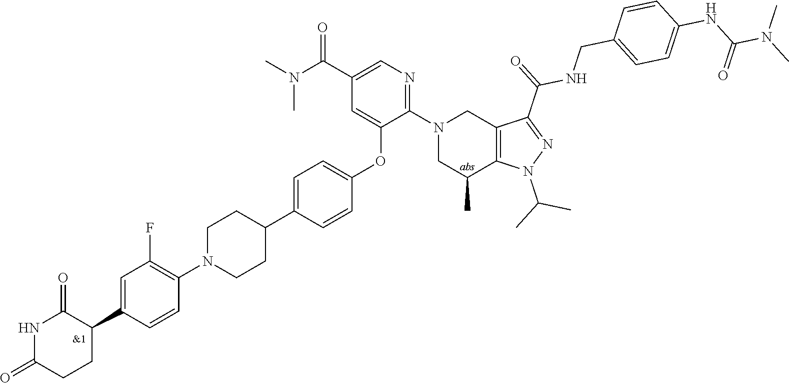 [SMILES: CC(C)n1nc(C(=O)NCc2ccc(NC(=O)N(C)C)cc2)c2c1[C@@H](C)CN(c1ncc(C(=O)N(C)C)cc1Oc1ccc(C3CCN(c4ccc([C@H]5CCC(=O)NC5=O)cc4F)CC3)cc1)C2] | (S)-5-(5-(dimethylcarbamoyl)-3- (4-(1-(4-((RS)-2,6-dioxopiperidin- 3-yl)-2-fluorophenyl)piperidin-4- yl)phenoxy)pyridin-2-yl)-N-(4- (3,3-dimethylureido)benzyl)-1- isopropyl-7-methyl-4,5,6,7- tetrahydro-1H-pyrazolo[4,3- c]pyridine-3-carboxamide |

### Pharmacology Assays

### PBMC STAT6 AlphaLISA Assay

Degrader potency (DC₅₀) and maximal efficacy (D_{MAX}) of test compounds was determined using human peripheral blood mononuclear cells (PBMCs)(Charles River #PB009C-3) AlphaLISA SureFire assay (Revvity #ALSU-TST6-A10K). This assay is a bead-based technology and sandwich immunoassays for the detection of total STAT6 levels.

Greiner 384-well white opaque, flat bottom, nonbinding assay plates (Corning #3574) were used, spotted with 20 nL of compound which had been serial diluted 1 in 4 in 100% DMSO for an 11-point concentration response curve, with duplicate points at each concentration. Top concentration being 0.5 mM (later diluted with cell suspension for a top concentration of 0.5 μM). High and zero percentage effect wells (HPE and ZPE) located in columns 1 and 24 were spotted with 20 nL of DMSO (Sigma #D2650) using the ECHO Acoustic Liquid Handler (Labcyte Echo 550 Series).

PBMCs were thawed into warmed Hanks Balanced Salt Solution w/calcium and magnesium (HBSS) and centrifuged at 300×g for 5 mins. Supernatant was discarded and PBMCs were resuspended in HBSS at 5e6 viable cells per mL (100K/well). Cell suspension was plated, on top of compound or DMSO control, in columns 2-24 using a multidrop liquid handler (ThermoScientific #24073296). HBSS alone was plated in column 1 (HPE). Cells were incubated overnight, with compound, for 24 hrs (with lid) at 37 °C (95% O₂: 6% CO₂).

After 24 hrs, using the AlphaLISA SureFire Kit reagents and a multidrop, cells were lysed with 5 μL/well of 5× lysis buffer. Plate was centrifuged at 1000 revolutions per minute (rpm) for 2 min, sealed with TopSeal-A PLUS (PerkinElmer #6050185) and put on a plate shaker for 1 hr at room temp.

After an hour, Total STAT6 AlphaLISA acceptor beads were vortexed, spun quickly, and used to prepare acceptor bead solution for each plate (1,645 μL RB1+1,645 μL RB2+140 μL warmed activation buffer (1:25-fold dilution in total volume)+70 μL acceptor beads (1:50-fold dilution in total volume)). Tube was mixed by inverting and 5 μL/well was added using a multidrop. Plate was centrifuged at 1000 rpm for 2 min, resealed, placed on a shaker for 2 min and left to incubate for 2 hrs at room temp. After acceptor bead incubation, all reagents were moved to a dark room under green light. Total STAT6 AlphaLISA donor beads were vortexed, spun quickly, and used to prepare donor bead solution for each plate (3,430 μL dilution buffer+70 μL donor beads (1:50-fold dilution in total volume)). Tube was mixed by inverting and 5 μL/well was added using a multidrop. Plate was resealed, placed on a shaker for 2 mins, wrapped in tinfoil, and then left to incubate in the dark, at room temp, overnight.

The following day, in the dark, seal is removed from the plate, and it was read using the EnVision Nexus (Revvity #HH36000002) under TSTAT6 AlphaLISA Screening protocol.

The percent (%) effect at each concentration of compound is calculated based on, and relative to, AlphaLISA signal in the HPE (100% effect) and ZPE (0% effect) wells. The concentrations and % effect values for tested compounds are plotted in Abase, and the concentration of compound required for 50% degradation (DC₅₀) was determined with a four-parameter logistic dose response equation. The endpoint value (DC₅₀) for reference compounds is evaluated in each experiment as a quality control measure. If the endpoint DC₅₀ was within 3-fold of the expected value and the ASMAX was +/−15% of expected value, then the experiment was deemed acceptable. Expected value is based off historical published data using the Endpoint compare tool. DC₅₀ and D_{MAX} values are shown in Table 5. NT mean Not Tested.

### Human Whole Blood (HWB) CCL17 TR-FRET Assay

The IC₅₀ (half-maximal concentration required for inhibition) of compounds for the inhibition of CCL17 secretion in peripheral human whole blood, stimulated through the IL-13-IL-13 receptor/IL-4 receptor pathway was evaluated with a TR-FRET sandwich ELISA CCL17 (TARC) Assay Kit (Bioauxilium).

Test compounds (solubilized and serially diluted in DMSO, in an 11 point, 3-fold dilution series in duplicate) were spotted, 80 nL per well, into a 384 well v-bottom polypropylene plate. Human whole blood was collected from a peripheral vein, 714 units of sodium heparin was added for every 50 mL of blood collected. Sixty μL per well of blood was added to the compound plate. The blood plate was incubated for two hours at 37 HC. After two hours, CCL7 production was stimulated with the addition of 20 μL per well of IL13 (R&D Systems) in HBSS with calcium and magnesium, with a final concentration of 10 ng/mL IL13. The plate containing blood, compound, and IL-13 was incubated at 37 °C for 48 hours, to allow for CCL17 production. After 48 hours, the plate was centrifuged at 1,500 rpm for 7 minutes, with no braking. The CCL17 TR-FRET Assay Kit detection reagent was prepared according to kit instructions and 4 μL per well of the antibody cocktail was added to a 384-well white low volume non-binding coated plate (Corning). Sixteen μL of serum from the centrifuged blood plate was transferred to the detection plate containing the TR-FRET antibody cocktail. The plate was centrifuged briefly, then covered and incubated 16 hours at room temperature. The plate was then read on an EnVision 2105 multilabel reader (PerkinElmer) with settings for TR-FRET Ratio=10,000× (fluorescence intensity 665 nm/fluorescence intensity 615 nm). IC₅₀ values were determined from this data using a 4-parameter fit algorithm and are shown in Table 6 along with the I_{MAX} values, where the IC₅₀ values are the statistical mean values of n number of samples as denoted by n(IC₅₀).

For any compound with an initial IC₅₀<1 nM, the compound curve plate is prepared using a different dilution method. The follow up submittal includes 11 points of 4-fold serial dilution, with the highest dose at 750 nM and the lowest at 0.71 μM. NT means Not Tested.

| | | | | HWB | HWB | |
|---|---|---|---|---|---|---|
| | PBMC | PBMC | | CCL17 | CCL17 | HWB |
| Example | DC₅₀ | D_{MAX} | PBMC | IC₅₀ | I_{MAX} | CCL17 |
| Number | (pM) | (%) | n | (pM) | (%) | n |
| E1 | 10.4 | 91.4 | 2 | 2827.0 | 100.3 | 3 |
| E2 | 2.9 | 92.2 | 3 | 113.7 | 100.9 | 3 |
| E3 | 9.9 | 87.6 | 6 | 686.3 | 100.3 | 4 |
| E4 | 36.5 | 89.6 | 3 | 893.5 | 100.3 | 5 |
| E5 | 10.0 | 90.1 | 3 | 182.9 | 99.3 | 4 |
| E6 | 15.8 | 93.4 | 2 | 240.6 | 98.4 | 2 |
| E7 | 3.0 | 89.5 | 3 | 392.6 | 101.9 | 2 |
| E8 | 181.6 | 85.4 | 3 | 103742.0 | 100.5 | 2 |
| E9 | 26.9 | 92.6 | 3 | 815.3 | 100.7 | 4 |
| E10 | 60.6 | 85.4 | 4 | 944.0 | 100.2 | 7 |
| E11 | 67.1 | 92.2 | 5 | 1803.3 | 99.8 | 7 |
| E12 | 42.5 | 93.1 | 3 | 234.0 | 103.1 | 1 |
| E13 | 201.9 | 80.4 | 9 | 30008.1 | 97.8 | 3 |
| E14 | 223.0 | 79.4 | 1 | 57018.1 | 102.3 | 3 |
| E15 | 1868.8 | 57.8 | 1 | 51943.7 | 99.9 | 3 |
| E16 | 211.9 | 91.3 | 1 | 1823.5 | 100.3 | 8 |
| E17 | 50.5 | 92.4 | 1 | 1557.5 | 99.8 | 5 |
| E18 | 14.5 | 89.1 | 10 | 126.7 | 99.4 | 3 |
| E19 | 207.3 | 60.4 | 1 | NT | NT | NT |
| E20 | 342.2 | 73.7 | 1 | NT | NT | NT |
| E21 | 49.5 | 92.9 | 1 | NT | NT | NT |
| E22 | 1635.1 | 66.0 | 1 | 35805.7 | 102.8 | 2 |
| E23 | 842.2 | 35.0 | 1 | NT | NT | NT |
| E24 | 492.6 | 56.0 | 1 | 131913.9 | 101.2 | 3 |
| E25 | 1120.2 | 43.1 | 1 | NT | NT | NT |
| E26 | 571.5 | 51.2 | 1 | NT | NT | NT |
| E27 | 12.4 | 95.2 | 2 | 388.4 | 100.3 | 1 |
| E28 | 818.8 | 80.4 | 1 | 33940.7 | 97.9 | 2 |
| E29 | 452.3 | 85.2 | 1 | 27075.9 | 99.4 | 2 |
| E30 | 272.0 | 65.6 | 1 | NT | NT | NT |
| E31 | 579.6 | 83.9 | 1 | 62460.2 | 98.1 | 2 |
| E32 | 800.1 | 66.0 | 1 | 146287.8 | 99.7 | 2 |
| E33 | 1349.8 | 72.6 | 1 | 88408.9 | 100.6 | 2 |
| E34 | 162.8 | 85.8 | 1 | 43400.5 | 94.5 | 2 |
| E35 | 2027.2 | 49.9 | 1 | NT | NT | NT |
| E36 | 459.1 | 79.9 | 1 | 45519.9 | 98.3 | 2 |
| E37 | 631.1 | 80.1 | 1 | 10088.2 | 96.9 | 3 |
| E38 | 1442.2 | 69.9 | 1 | NT | NT | NT |
| E39 | 1294.0 | 53.3 | 1 | NT | NT | NT |
| E40 | 882.5 | 85.2 | 1 | 9170.6 | 96.6 | 3 |
| E41 | 117.9 | 80.0 | 1 | 7803.3 | 98.6 | 3 |
| E42 | 19.1 | 90.7 | 2 | 1639.3 | 98.4 | 4 |
| E43 | 691.6 | 44.4 | 1 | NT | NT | NT |
| E44 | 535.1 | 77.8 | 1 | 18122.0 | 96.8 | 2 |
| E45 | 738.2 | 63.7 | 1 | NT | NT | NT |
| E46 | 1133.8 | 88.0 | 1 | NT | NT | NT |
| E47 | 33.7 | 79.8 | 2 | 4174.5 | 98.8 | 5 |
| E48 | 7.5 | 90.6 | 3 | 224.0 | 99.8 | 4 |
| E49 | 15.2 | 88.7 | 1 | <677.7 | 99.5 | 6 |
| E50 | 5.0 | 93.7 | 3 | 229.5 | 100.4 | 2 |
| E51 | 548.5 | 85.6 | 1 | 8376.8 | 98.1 | 3 |
| E52 | 46.8 | 86.1 | 1 | 7482.6 | 101.1 | 2 |
| E53 | 1318.9 | 50.0 | 1 | 23010.4 | 97.8 | 3 |
| E54 | 1869.2 | 71.0 | 1 | 61924.8 | 98.7 | 3 |
| E55 | 66.9 | 93.9 | 1 | 4137.4 | 100.1 | 3 |
| E56 | 17.3 | 87.4 | 2 | 7566.2 | 99.3 | 4 |
| E57 | 2143.6 | 63.0 | 1 | 36628.7 | 96.1 | 3 |
| E58 | 381.8 | 75.2 | 1 | 8760.1 | 98.8 | 3 |
| E59 | 203.1 | 48.7 | 1 | 6540.7 | 97.7 | 3 |
| E60 | 14.1 | 91.6 | 52 | 384.7 | 99.3 | 5 |
| E61 | 12.3 | 83.5 | 2 | 948.1 | 100.2 | 3 |
| E62 | 7.4 | 91.2 | 3 | 484.3 | 99.3 | 3 |
| E63 | 2178.5 | 60.8 | 1 | 1959.8 | 101.1 | 3 |
| E64 | 6.8 | 93.1 | 50 | 246.5 | 99.2 | 3 |
| E65 | 9.7 | 92.3 | 2 | 746.3 | 99.3 | 3 |
| E66 | 2.4 | 90.2 | 1 | 149.6 | 99.5 | 2 |
| E67 | 16.0 | 87.4 | 2 | 970.8 | 101.6 | 2 |
| E68 | 92.8 | 77.4 | 2 | 2405.7 | 101.0 | 2 |
| E69 | 90.0 | 72.8 | 2 | 6151.4 | 100.4 | 2 |
| E70 | 80.6 | 85.2 | 2 | 2173.3 | 100.3 | 3 |
| E71 | 741.4 | 67.3 | 1 | 5107.3 | 96.9 | 2 |
| E72 | 28.5 | 88.2 | 2 | 1934.0 | 100.6 | 2 |
| E73 | 2.5 | 92.4 | 3 | 206.3 | 100.6 | 2 |
| E74 | 3.3 | 93.0 | 3 | 268.1 | 100.4 | 2 |
| E75 | 6.2 | 90.9 | 1 | 380.0 | 101.0 | 2 |
| E76 | 3.7 | 92.7 | 3 | 181.2 | 101.1 | 2 |
| E77 | 13.4 | 83.7 | 2 | 924.5 | 98.1 | 2 |
| E78 | 11.1 | 89.8 | 2 | 190.4 | 101.3 | 2 |
| E79 | 4.2 | 92.4 | 3 | 144.9 | 100.2 | 2 |
| E80 | 7.6 | 91.0 | 2 | 968.0 | 98.1 | 2 |
| E81 | 314.6 | 93.3 | 3 | 371.3 | 100.9 | 2 |
| E82 | 5.1 | 90.0 | 3 | 287.7 | 100.5 | 2 |
| E83 | 15.4 | 81.2 | 2 | 1126.9 | 101.4 | 2 |
| E84 | 2.3 | 91.6 | 3 | 79.7 | 101.4 | 2 |
| E85 | 8.4 | 89.7 | 2 | 319.4 | 101.1 | 2 |
| E86 | 6.4 | 91.7 | 3 | 340.5 | 99.0 | 1 |
| E87 | 12.5 | 92.3 | 2 | NT | NT | NT |
| E88 | 22.8 | 84.2 | 3 | 952.9 | 100.6 | 2 |
| E89 | 1931.4 | 76.2 | 2 | 73141.1 | 96.2 | 1 |
| E90 | 54.0 | 91.1 | 1 | NT | NT | NT |
| E91 | 80.3 | 70.5 | 2 | 11409.4 | 98.5 | 5 |
| E92 | 70.1 | 93.4 | 1 | 2353.1 | 99.3 | 2 |
| E93 | 13.5 | 89.9 | 2 | 1617.9 | 99.5 | 2 |
| E94 | 18.0 | 85.6 | 2 | 3296.9 | 98.8 | 2 |
| E95 | 138.9 | 64.3 | 1 | 8929.4 | 100.2 | 2 |
| E96 | 262.3 | 77.2 | 1 | 9581.9 | 98.8 | 2 |
| E97 | 233.5 | 69.5 | 1 | 8533.8 | 100.0 | 2 |
| E98 | 227.5 | 63.8 | 1 | 15641.3 | 98.1 | 2 |
| E99 | 26.6 | 88.4 | 3 | 1514.8 | 99.8 | 2 |
| E100 | 68.3 | 92.4 | 2 | 1380.1 | 99.1 | 2 |
| E101 | 103.6 | 81.9 | 2 | 9089.1 | 98.7 | 3 |
| E102 | 122.3 | 69.8 | 1 | 9107.0 | 98.4 | 2 |
| E103 | 23.2 | 81.0 | 2 | 4622.2 | 99.1 | 2 |
| E104 | 431.0 | 81.7 | 1 | 15047.6 | 99.7 | 3 |
| E105 | 10.1 | 92.1 | 3 | 1299.8 | 100.4 | 2 |
| E106 | 21.4 | 86.0 | 3 | 1400.1 | 100.8 | 2 |
| E107 | 72.8 | 73.1 | 2 | 5016.1 | 99.1 | 4 |
| E108 | 13.5 | 88.0 | 3 | 1265.2 | 98.9 | 5 |
| E109 | 34.6 | 82.6 | 3 | 1974.3 | 99.7 | 5 |
| E110 | 458.3 | 64.8 | 1 | 22132.0 | 98.2 | 3 |
| E111 | 112.5 | 67.2 | 2 | 7551.2 | 99.4 | 4 |
| E112 | 294.2 | 53.7 | 1 | 8125.5 | 99.5 | 5 |
| E113 | 47.1 | 87.9 | 2 | 2763.2 | 99.1 | 5 |
| E114 | 46.8 | 86.8 | 2 | 3878.9 | 99.4 | 3 |
| E115 | 1130.0 | 84.6 | 1 | 15692.1 | 99.7 | 2 |
| E116 | 200.6 | 95.1 | 1 | 3038.8 | 100.0 | 3 |
| E117 | 576.6 | 96.8 | 1 | 2834.0 | 100.1 | 3 |
| E118 | 188.7 | 87.4 | 3 | 5711.8 | 100.1 | 1 |
| E119 | 15.6 | 80.1 | 3 | 1427.4 | 97.2 | 1 |
| E120 | 15.3 | 81.9 | 2 | 1095.4 | 99.9 | 2 |
| E121 | 59.7 | 88.0 | 4 | 3646.8 | 99.1 | 4 |
| E122 | 10.8 | 92.0 | 3 | 453.0 | 100.5 | 2 |
| E123 | 28.0 | 85.8 | 2 | 3435.9 | 82.0 | 4 |
| E124 | 59.1 | 83.7 | 2 | 7834.8 | 97.3 | 2 |
| E125 | 21.2 | 89.8 | 2 | 1056.4 | 100.0 | 2 |
| E126 | 163.7 | 80.3 | 2 | 3026.2 | 98.2 | 2 |
| E127 | 108.5 | 63.3 | 2 | 4781.0 | 98.3 | 2 |
| E128 | 33.0 | 84.4 | 2 | 1151.5 | 102.3 | 3 |
| E129 | 9.9 | 90.7 | 2 | 638.5 | 100.3 | 2 |
| E130 | 32.1 | 81.8 | 2 | 3287.1 | 97.3 | 2 |
| E131 | 3.9 | 91.5 | 1 | NT | NT | NT |
| E132 | 28.6 | 86.8 | 2 | 1773.9 | 98.1 | 2 |
| E133 | 77.4 | 83.1 | 2 | 5008.7 | 97.6 | 2 |
| E134 | 285.9 | 64.8 | 2 | 5471.1 | 103.6 | 1 |
| E135 | 132.2 | 80.1 | 2 | 9426.6 | 95.9 | 2 |
| E136 | 35.4 | 92.9 | 2 | 1156.4 | 102.2 | 1 |
| E137 | 336.0 | 76.2 | 1 | 21235.1 | 98.4 | 2 |
| E138 | 98.6 | 78.4 | 1 | 7747.6 | 100.4 | 2 |
| E139 | 190.2 | 85.2 | 2 | 6947.7 | 99.6 | 5 |
| E140 | 28.8 | 93.4 | 2 | 1775.6 | 99.6 | 3 |
| E141 | 20.7 | 93.0 | 1 | NT | NT | NT |
| E142 | 215.8 | 75.2 | 2 | 30880.4 | 98.0 | 2 |
| E143 | 721.9 | 62.4 | 1 | 113438.3 | 98.4 | 2 |
| E144 | 18.7 | 90.9 | 2 | 791.7 | 100.5 | 7 |
| E145 | 31.8 | 89.3 | 2 | 1827.1 | 99.6 | 2 |
| E146 | 202.1 | 83.0 | 1 | 3800.8 | 97.9 | 2 |
| E147 | 14.1 | 88.7 | 2 | 1111.7 | 101.0 | 3 |
| E148 | 236.6 | 68.1 | 1 | 6588.9 | 98.9 | 2 |
| E149 | 20.2 | 84.1 | 2 | 1187.2 | 98.9 | 2 |
| E150 | 10.0 | 91.9 | 2 | 435.6 | 101.0 | 4 |
| E151 | 78.8 | 79.8 | 1 | 2012.9 | 100.4 | 3 |
| E152 | 72.1 | 85.0 | 1 | 2407.6 | 100.2 | 2 |
| E153 | 14.7 | 87.7 | 2 | 710.3 | 101.1 | 3 |
| E154 | 15.2 | 89.6 | 2 | NT | NT | NT |
| E155 | 7.0 | 88.7 | 2 | 432.5 | 100.7 | 3 |
| E156 | 6.8 | 92.5 | 2 | 140.6 | 101.6 | 3 |
| E157 | 6.8 | 91.4 | 2 | 208.6 | 99.8 | 1 |
| E158 | 29.8 | 84.6 | 2 | 1651.0 | 100.7 | 3 |
| E159 | 15.9 | 88.4 | 2 | 333.2 | 99.6 | 3 |
| E160 | 10.3 | 94.5 | 3 | 806.0 | 101.5 | 3 |
| E161 | 11.5 | 88.5 | 2 | 1001.2 | 99.6 | 3 |
| E162 | 21.7 | 63.2 | 2 | 2763.9 | 96.7 | 3 |
| E163 | 27.7 | 84.5 | 2 | 590.8 | 101.7 | 3 |
| E164 | 5.7 | 92.7 | 2 | 181.5 | 102.2 | 3 |
| E165 | 4.0 | 93.3 | 2 | 79.3 | 100.1 | 3 |
| E166 | 21.5 | 92.2 | 2 | 310.7 | 103.3 | 1 |
| E167 | 258.2 | 61.2 | 2 | 9307.3 | 95.1 | 3 |
| E168 | 26.2 | 93.2 | 2 | 1518.6 | 98.8 | 3 |
| E169 | 148.2 | 87.0 | 2 | 16751.7 | 87.3 | 2 |
| E170 | 623.1 | 67.3 | 1 | 55803.5 | 98.8 | 2 |
| E171 | 17.1 | 94.0 | 2 | 556.1 | 99.3 | 1 |
| E172 | 7.8 | 96.6 | 2 | 898.9 | 99.0 | 1 |
| E173 | 1172.1 | 92.4 | 1 | 27329.8 | 78.7 | 1 |
| E174 | 217.3 | 38.0 | 2 | 27972.3 | 95.4 | 2 |
| E175 | 73.2 | 84.3 | 2 | 3600.3 | 98.8 | 2 |
| E176 | 536.1 | 90.0 | 1 | 8610.0 | 99.0 | 1 |
| E177 | 19.5 | 80.9 | 3 | 2092.3 | 99.9 | 2 |
| E178 | 250.1 | 79.3 | 1 | 6349.3 | 101.2 | 2 |
| E179 | 20.8 | 93.1 | 2 | 2319.7 | 101.7 | 2 |
| E180 | 205.0 | 41.4 | 1 | 205407.1 | 93.8 | 2 |
| E181 | 57.5 | 94.1 | 1 | NT | NT | NT |
| E182 | 14.8 | 95.4 | 2 | 262.0 | 101.8 | 1 |
| E183 | 4.6 | 93.6 | 2 | 44.2 | 100.8 | 1 |
| E184 | 83.3 | 93.6 | 2 | 1766.5 | 102.2 | 2 |
| E185 | 21.8 | 96.1 | 2 | 3415.2 | 104.9 | 2 |
| E187 | 28.5 | 95.1 | 2 | NT | NT | NT |
| E188 | 24.6 | 94.8 | 1 | NT | NT | NT |
| E189 | NT | NT | NT | NT | NT | NT |
| E190 | 60.3 | 76.4 | 1 | <1184.1 | 100.6 | 2 |
| E191 | 7.5 | 94.4 | 2 | 25.8 | 105.2 | 1 |
| E192 | 20.1 | 91.2 | 1 | 937.1 | 102.9 | 2 |
| E193 | 20.2 | 93.4 | 1 | 496.2 | 102.8 | 1 |
| E194 | 36.3 | 87.3 | 1 | 863.3 | 102.5 | 2 |
| E195 | 99.6 | 88.1 | 2 | 999.7 | 104.4 | 2 |
| E196 | 25.3 | 92.7 | 2 | 442.1 | 101.4 | 2 |
| E197 | 92.4 | 86.0 | 2 | <535.9 | 100.0 | 2 |
| E198 | 31.1 | 87.3 | 1 | <510.0 | NT | 1 |
| E199 | 1942.0 | 95.6 | 1 | 9463.1 | 100.6 | 2 |
| E200 | 103.8 | 92.9 | 4 | 1261.4 | 98.3 | 2 |
| E201 | 179.2 | 65.0 | 2 | 6457.9 | 89.3 | 3 |
| E202 | 323.3 | 90.4 | 2 | 2759.7 | 99.4 | 3 |
| E203 | 2110.7 | 97.6 | 1 | 12247.3 | 100.8 | 4 |
| E204 | 171.0 | 92.3 | 2 | 3601.5 | 98.4 | 2 |
| E205 | >500000.0 | NT | 1 | 106201.6 | 85.1 | 3 |
| E206 | 2031.5 | 77.2 | 1 | 46239.5 | 99.8 | 2 |
| E207 | 89.2 | 95.1 | 2 | 1868.2 | 99.2 | 2 |
| E208 | 103.4 | 95.5 | 2 | 1614.2 | 101.3 | 3 |
| E209 | 15.4 | 97.3 | 3 | <606.1 | 101.4 | 4 |
| E210 | 685.1 | 83.8 | 1 | 2211.2 | 99.9 | 1 |
| E211 | 154.6 | 91.9 | 2 | <985.1 | 97.8 | 2 |
| E212 | 9.3 | 97.3 | 2 | 78.5 | 104.6 | 2 |
| E213 | 3.7 | 95.8 | 2 | 1731.6 | 101.0 | 2 |
| E214 | 13.3 | 97.3 | 2 | 369.0 | 101.4 | 2 |
| E215 | 21.5 | 93.5 | 7 | 2835.0 | 99.3 | 3 |
| E216 | 25.6 | 94.5 | 3 | <1064.5 | 99.1 | 4 |
| E217 | 13.7 | 95.2 | 3 | 1224.4 | 99.2 | 5 |
| E218 | 29.7 | 93.7 | 3 | 1701.1 | 99.1 | 4 |
| E219 | 6.5 | 95.6 | 7 | 206.9 | 100.3 | 2 |
| E220 | 3.5 | 97.3 | 2 | 119.5 | 97.2 | 1 |
| E221 | 70.9 | 89.7 | 2 | 1661.5 | 100.4 | 2 |
| E222 | 38.1 | 93.4 | 6 | 849.3 | 99.9 | 3 |
| E223 | 421.4 | 83.4 | 2 | 4694.1 | 97.5 | 3 |
| E224 | 33.8 | 89.2 | 2 | 3249.0 | 100.5 | 2 |
| E225 | 100.0 | 94.0 | 3 | 1900.7 | 97.4 | 5 |
| E226 | 126.5 | 91.5 | 1 | 8720.1 | 99.8 | 2 |
| E227 | 18.8 | 96.6 | 2 | 1357.2 | 98.3 | 3 |
| E228 | 6.1 | 96.5 | 2 | <510.0 | NT | 1 |
| E229 | 9.7 | 95.0 | 2 | 505.4 | 99.3 | 2 |
| E230 | 24.7 | 95.0 | 3 | 2044.6 | 98.1 | 3 |
| E231 | 358.6 | 89.3 | 2 | 5882.5 | 102.0 | 2 |
| E232 | 3.0 | 96.9 | 2 | 171.3 | 100.7 | 1 |
| E233 | 3.2 | 97.5 | 2 | <520.0 | 97.0 | 2 |
| E234 | 8.0 | 97.0 | 2 | 286.9 | 100.0 | 1 |
| E235 | 11.5 | 95.9 | 2 | 905.8 | 100.3 | 1 |
| E236 | 12.4 | 96.9 | 3 | 1042.4 | 100.4 | 2 |
| E237 | 12.0 | 97.5 | 2 | <645.4 | 97.1 | 2 |
| E238 | 15.8 | 96.4 | 2 | 1878.2 | 94.9 | 2 |
| E239 | 19.1 | 97.5 | 2 | 1567.4 | 96.1 | 2 |
| E240 | 30.8 | 96.8 | 2 | <694.5 | 100.4 | 2 |
| E241 | 33.9 | 91.8 | 2 | <863.5 | 100.7 | 2 |
| E242 | 31.8 | 88.4 | 2 | <664.4 | 95.9 | 2 |
| E243 | 21.0 | 96.0 | 2 | 1015.2 | 99.4 | 2 |
| E244 | 27.1 | 97.6 | 2 | <619.9 | 100.6 | 2 |
| E245 | 36.3 | 96.3 | 2 | <742.5 | 100.0 | 2 |
| E246 | 51.6 | 90.5 | 2 | 992.5 | 99.0 | 1 |
| E247 | 907.8 | 90.5 | 2 | 10046.1 | 97.8 | 3 |
| E248 | 59.8 | 95.4 | 6 | 2286.5 | 99.1 | 3 |
| E249 | 71.4 | 90.8 | 2 | 1685.3 | 98.3 | 2 |
| E250 | 20.8 | 96.5 | 2 | 672.4 | 98.6 | 3 |
| E251 | 15.5 | 96.2 | 2 | <510.0 | NT | 1 |
| E252 | 23.7 | 95.3 | 2 | <510.0 | NT | 1 |
| E253 | 36.6 | 89.5 | 2 | 843.4 | 99.4 | 1 |
| E254 | 471.5 | 83.1 | 2 | 2037.2 | 98.0 | 1 |
| E255 | 2.1 | 97.4 | 2 | 73.3 | 100.6 | 1 |
| E256 | 710.7 | 89.2 | 1 | 7499.5 | 99.6 | 1 |
| E257 | 11.1 | 95.2 | 2 | 274.9 | 100.2 | 1 |
| E258 | 123.2 | 89.6 | 2 | 2305.0 | 98.5 | 2 |
| E259 | 128.3 | 95.3 | 2 | 14589.0 | 99.5 | 2 |
| E260 | 213.0 | 89.0 | 2 | 4472.1 | 99.2 | 5 |
| E261 | 12.5 | 94.5 | 2 | <675.9 | 100.7 | 2 |
| E262 | 86.6 | 89.8 | 2 | 1359.4 | 99.7 | 1 |
| E263 | 38.0 | 91.0 | 2 | 1097.4 | 99.9 | 2 |
| E264 | 89.4 | 96.3 | 2 | 4193.0 | 100.0 | 4 |
| E265 | 68.3 | 91.0 | 2 | 1717.3 | 101.1 | 1 |
| E266 | 13.5 | 95.5 | 2 | 174.7 | 100.2 | 1 |
| E267 | 7.0 | 98.2 | 2 | 251.2 | 99.4 | 1 |
| E268 | 20.7 | 95.7 | 2 | 473.4 | 99.6 | 1 |
| E269 | 95.1 | 92.5 | 2 | 1474.7 | 101.1 | 1 |
| E270 | 148.0 | 92.1 | 2 | 3841.1 | 100.8 | 1 |
| E271 | 396.0 | 87.9 | 2 | 6474.3 | 98.8 | 1 |
| E272 | 962.0 | 88.7 | 1 | 26002.6 | 96.6 | 1 |
| E273 | 10.9 | 93.5 | 5 | 220.8 | 99.9 | 1 |
| E274 | 16.9 | 93.8 | 2 | 349.8 | 99.2 | 1 |
| E275 | 73.5 | 88.4 | 2 | 2050.6 | 99.6 | 1 |
| E276 | 37.2 | 94.0 | 2 | 707.3 | 98.8 | 2 |
| E277 | 11.8 | 96.9 | 3 | 53.2 | 100.6 | 1 |
| E278 | 51.8 | 96.1 | 3 | 592.4 | 100.2 | 1 |
| E279 | 41.3 | 92.1 | 2 | 561.9 | 103.3 | 1 |
| E280 | 717.1 | 68.1 | 1 | 6450.9 | 99.6 | 1 |
| E281 | 249.1 | 90.8 | 2 | 3424.9 | 99.9 | 2 |
| E282 | 18.0 | 93.6 | 2 | 354.4 | 99.9 | 1 |
| E283 | 23.2 | 95.2 | 2 | 588.7 | 100.1 | 1 |
| E284 | 647.0 | 91.5 | 2 | 7623.5 | 99.6 | 3 |
| E285 | 14.2 | 92.2 | 5 | 637.1 | 99.7 | 1 |
| E286 | 65.9 | 90.4 | 2 | 2353.7 | 101.0 | 1 |
| E287 | 33.4 | 95.4 | 2 | 551.3 | 100.7 | 1 |
| E288 | 97.0 | 90.3 | 2 | 3734.2 | 99.4 | 1 |
| E289 | 232.1 | 88.8 | 2 | 8891.9 | 100.3 | 1 |
| E290 | 976.0 | 88.6 | 1 | 17958.4 | 101.3 | 1 |
| E291 | 23.8 | 96.4 | 2 | 330.5 | 99.1 | 1 |
| E292 | 33.9 | 92.8 | 2 | 229.5 | 100.6 | 1 |
| E293 | 11.4 | 97.4 | 3 | 188.9 | 101.5 | 1 |
| E294 | 38.9 | 91.3 | 2 | 418.4 | 100.8 | 1 |
| E295 | 183.1 | 90.3 | 2 | 1129.5 | 101.0 | 1 |
| E296 | 184.6 | 77.8 | 2 | 4038.6 | 99.8 | 1 |
| E297 | 3.9 | 98.2 | 2 | 84.5 | 100.1 | 1 |
| E298 | 3.7 | 98.6 | 3 | 46.1 | 101.9 | 1 |
| E299 | 6.9 | 93.9 | 2 | 175.4 | 101.1 | 2 |
| E300 | 10.6 | 98.1 | 2 | 617.3 | 101.2 | 1 |
| E301 | 70.2 | 91.7 | 2 | 720.7 | 99.4 | 1 |
| E302 | 19.1 | 97.0 | 2 | 1044.2 | 100.9 | 1 |
| E303 | 39.5 | 87.7 | 2 | 474.2 | 99.6 | 1 |
| E304 | 87.2 | 87.0 | 2 | 3629.7 | 98.6 | 1 |
| E305 | 141.7 | 92.6 | 2 | 1938.0 | 101.4 | 1 |
| E306 | 223.3 | 74.1 | 2 | 6452.7 | 94.0 | 1 |
| E307 | 382.3 | 90.2 | 2 | 8071.8 | 100.1 | 1 |
| E308 | 315.8 | 70.1 | 2 | 5305.3 | 92.0 | 1 |
| E309 | 524.4 | 74.5 | 2 | 6892.4 | 94.8 | 1 |
| E310 | 959.1 | 71.8 | 1 | NT | NT | NT |
| E311 | 25.4 | 87.7 | 2 | 415.9 | 99.9 | 1 |
| E312 | 28.6 | 94.6 | 2 | 150.1 | 100.9 | 1 |
| E313 | 41.0 | 89.7 | 2 | 1998.4 | 97.8 | 2 |
| E314 | 297.1 | 91.8 | 2 | 1348.1 | 98.5 | 1 |
| E315 | 30.8 | 95.6 | 3 | 322.0 | 100.1 | 1 |
| E316 | 48.0 | 85.5 | 2 | 642.0 | 101.0 | 1 |
| E317 | 8.0 | 97.6 | 2 | 69.1 | 102.5 | 1 |
| E318 | 75.5 | 87.3 | 2 | <600.1 | 99.4 | 2 |
| E319 | 121.0 | 89.8 | 2 | 1279.4 | 98.9 | 4 |
| E320 | 42.6 | 91.4 | 2 | 3050.7 | 98.5 | 3 |
| E321 | 47.9 | 91.8 | 2 | 1418.6 | 99.6 | 3 |
| E322 | 78.6 | 95.9 | 3 | <899.9 | 97.7 | 2 |
| E323 | 90.7 | 96.3 | 2 | 3204.0 | 98.5 | 4 |
| E324 | 47.9 | 89.8 | 2 | 5089.7 | 96.6 | 2 |
| E325 | 119.8 | 96.9 | 2 | <1166.4 | 99.8 | 2 |
| E326 | 63.2 | 87.3 | 2 | 1697.5 | 100.2 | 1 |
| E327 | 68.6 | 89.7 | 2 | 2216.8 | 101.2 | 2 |
| E328 | 124.5 | 90.4 | 2 | 5760.7 | 97.4 | 2 |
| E329 | 56.1 | 90.8 | 2 | 2958.8 | 98.7 | 2 |
| E330 | 76.5 | 89.0 | 2 | 2264.7 | 100.1 | 1 |
| E331 | 237.0 | 67.7 | 2 | 7467.6 | 98.2 | 2 |
| E332 | 286.9 | 86.9 | 2 | 16635.2 | 99.2 | 1 |
| E333 | 264.6 | 87.4 | 2 | 4940.8 | 101.4 | 2 |
| E334 | 300.0 | 76.8 | 2 | 6484.5 | 99.6 | 1 |
| E335 | 235.9 | 87.4 | 2 | 2078.2 | 98.9 | 2 |
| E336 | 399.1 | 88.7 | 2 | 19917.7 | 100.8 | 2 |
| E337 | 394.8 | 89.1 | 2 | 17444.4 | 98.2 | 2 |
| E338 | 577.4 | 88.4 | 2 | 13411.2 | 97.5 | 2 |
| E339 | 605.8 | 84.6 | 2 | 24615.3 | 96.4 | 2 |
| E340 | 345.5 | 88.2 | 2 | 4305.0 | 97.8 | 2 |
| E341 | 500.0 | 88.0 | 2 | 30908.0 | 96.2 | 2 |
| E342 | 601.2 | 88.9 | 1 | 18403.2 | 97.1 | 1 |
| E343 | 849.4 | 56.6 | 1 | 10198.7 | 99.4 | 1 |
| E344 | 951.0 | 55.4 | 1 | 65417.9 | 90.2 | 1 |
| E345 | 12.4 | 92.2 | 2 | 466.3 | 99.8 | 1 |
| E346 | 77.3 | 92.7 | 2 | 1649.2 | 100.0 | 1 |
| E347 | 121.8 | 82.4 | 2 | 5010.6 | 95.5 | 1 |
| E348 | 364.4 | 84.6 | 2 | 4986.2 | 98.6 | 2 |
| E349 | 494.2 | 56.7 | 2 | 57528.9 | 85.9 | 1 |
| E350 | 373.9 | 88.7 | 2 | 10847.0 | 99.8 | 2 |
| E351 | 904.1 | 85.2 | 2 | 19932.8 | 100.8 | 1 |
| E352 | 794.4 | 90.3 | 1 | 18381.0 | 99.2 | 1 |
| E353 | 857.5 | 73.2 | 1 | 9384.4 | 100.3 | 1 |
| E354 | 888.2 | 85.5 | 1 | 28320.2 | 99.1 | 2 |
| E355 | 17.1 | 92.3 | 2 | 396.0 | 100.0 | 1 |
| E356 | 98.1 | 94.4 | 2 | 940.8 | 102.5 | 1 |
| E357 | 639.6 | 90.0 | 2 | 5187.2 | 100.6 | 1 |
| E358 | 103.1 | 96.5 | 2 | 2033.9 | 98.3 | 3 |
| E359 | 844.4 | 93.0 | 1 | 48024.1 | 95.8 | 2 |
| E360 | 324.9 | 89.8 | 2 | 21972.5 | 99.8 | 1 |
| E361 | 593.6 | 80.3 | 2 | 34592.7 | 95.9 | 1 |
| E362 | 176.9 | 75.7 | 2 | 2344.9 | 64.9 | 1 |
| E363 | 75.5 | 96.1 | 2 | 3768.2 | 100.0 | 1 |
| E364 | 67.1 | 89.3 | 2 | 3707.2 | 99.1 | 1 |
| E365 | 880.2 | 84.4 | 1 | 17412.5 | 99.9 | 1 |
| E366 | 243.9 | 90.2 | 2 | 8576.3 | 99.3 | 1 |
| E367 | 98.5 | 92.4 | 2 | 1432.5 | 106.7 | 1 |
| E368 | 19.3 | 91.9 | 2 | 371.0 | 101.9 | 1 |
| E369 | 14.9 | 97.9 | 2 | 250.9 | 100.4 | 1 |
| E370 | 26.5 | 92.2 | 2 | 333.2 | 101.7 | 1 |
| E371 | 663.6 | 91.4 | 2 | 2852.3 | 102.0 | 1 |
| E372 | 1081.0 | 96.0 | 1 | 12818.1 | 107.2 | 1 |
| E373 | 19.6 | 96.7 | 2 | 412.3 | 100.7 | 1 |
| E374 | 49.0 | 88.4 | 2 | 308.5 | 106.3 | 1 |
| E375 | 90.2 | 89.0 | 2 | 858.0 | 100.6 | 1 |
| E376 | 10.0 | 98.1 | 2 | 2517.8 | 101.8 | 1 |
| E377 | 68.1 | 92.6 | 2 | 2408.6 | 101.0 | 1 |
| E378 | 30.7 | 97.3 | 3 | 1104.9 | 100.0 | 1 |
| E379 | 6.7 | 86.3 | 3 | 223.6 | 100.0 | 1 |
| E380 | 67.7 | 92.2 | 2 | 1208.5 | 99.6 | 1 |
| E381 | 12.1 | 97.6 | 3 | 288.5 | 101.6 | 1 |
| E382 | 43.8 | 83.9 | 3 | 1932.4 | 98.9 | 1 |
| E383 | 11.1 | 97.3 | 2 | 117.7 | 100.2 | 1 |
| E384 | 60.3 | 82.8 | 2 | 12649.5 | 99.7 | 1 |
| E385 | <34.9 | 96.8 | 3 | 21585.9 | 99.4 | 1 |
| E386 | 46.8 | 80.4 | 2 | 2402.0 | 99.9 | 1 |
| E387 | 63.2 | 85.7 | 2 | 2685.6 | 101.5 | 1 |
| E388 | 239.0 | 78.6 | 2 | 11206.1 | 99.3 | 1 |
| E389 | 270.3 | 82.9 | 2 | 21356.0 | 100.0 | 1 |
| E390 | 45.1 | 82.3 | 2 | 1297.0 | 99.5 | 1 |
| E391 | 69.3 | 85.6 | 2 | 5713.8 | 99.5 | 1 |
| E392 | 92.0 | 80.4 | 2 | 2022.9 | 100.3 | 1 |
| E393 | 979.9 | 87.1 | 1 | 82751.9 | 98.7 | 1 |
| E394 | 8.6 | 87.5 | 2 | 1180.9 | 99.6 | 1 |
| E395 | 71.3 | 90.9 | 2 | 1813.7 | 99.9 | 1 |
| E396 | 179.5 | 88.7 | 2 | 9925.7 | 100.4 | 1 |
| E397 | 395.7 | 89.9 | 2 | 21030.9 | 98.9 | 1 |
| E398 | 2.3 | 97.3 | 2 | 167.6 | 98.5 | 1 |
| E399 | 4.7 | 92.7 | 2 | 95.3 | 100.2 | 1 |
| E400 | 6.2 | 98.2 | 2 | 1581.4 | 99.7 | 1 |
| E401 | 63.2 | 97.7 | 3 | 2732.1 | 100.4 | 1 |
| E402 | 281.0 | 82.1 | 2 | 2855.8 | 98.5 | 1 |
| E403 | 25.1 | 96.7 | 3 | 325.3 | 101.1 | 1 |
| E404 | 22.9 | 80.2 | 2 | <568.0 | 100.3 | 2 |
| E405 | 7.3 | 92.1 | 2 | 219.5 | 100.7 | 1 |
| E406 | 385.9 | 88.2 | 2 | 21210.1 | 100.8 | 1 |
| E407 | 249.0 | 87.6 | 2 | 7626.1 | 99.9 | 1 |
| E408 | 9.2 | 96.5 | 3 | 141.3 | 101.2 | 1 |
| E409 | 30.5 | 83.4 | 2 | 407.5 | 100.7 | 1 |
| E410 | 29.5 | 93.6 | 4 | <664.2 | 99.6 | 2 |
| E411 | 56.3 | 90.7 | 2 | 2346.7 | 99.7 | 1 |
| E412 | 1.0 | 97.6 | 2 | 160.0 | 100.9 | 1 |
| E413 | 3.3 | 94.8 | 2 | 131.0 | 98.9 | 1 |
| E414 | 18.8 | 91.4 | 2 | 418.7 | 99.7 | 1 |
| E415 | 89.9 | 88.9 | 2 | 2447.8 | 97.7 | 1 |
| E416 | 451.8 | 73.8 | 2 | 15102.2 | 99.7 | 1 |
| E417 | 66.8 | 89.0 | 3 | 962.7 | 99.0 | 1 |
| E418 | 120.9 | 97.1 | 3 | 5938.3 | 99.7 | 3 |
| E419 | 314.4 | 90.4 | 2 | 5986.4 | 98.3 | 2 |
| E420 | 334.5 | 76.0 | 2 | 7315.7 | 99.0 | 1 |
| E421 | 12.7 | 91.9 | 3 | 151.4 | 99.4 | 1 |
| E422 | 2.7 | 97.8 | 2 | 90.6 | 100.1 | 1 |
| E423 | 10.2 | 88.2 | 2 | 165.3 | 100.3 | 1 |
| E424 | 10.6 | 97.7 | 2 | 581.6 | 101.3 | 1 |
| E425 | 171.4 | 73.7 | 2 | 4123.3 | 99.5 | 1 |
| E426 | 12.9 | 97.5 | 3 | 131.9 | 99.5 | 1 |
| E427 | 15.8 | 97.0 | 3 | 345.5 | 100.2 | 1 |
| E428 | 23.9 | 97.0 | 2 | 184.9 | 99.5 | 1 |
| E429 | 103.2 | 93.0 | 2 | 1045.9 | 102.4 | 1 |
| E430 | 119.0 | 97.1 | 2 | 353.2 | 100.3 | 1 |
| E431 | 9.6 | 97.6 | 3 | 80.7 | 100.6 | 1 |
| E432 | 16.2 | 95.7 | 2 | 227.0 | 103.2 | 1 |
| E433 | 17.6 | 86.9 | 3 | 166.2 | 100.2 | 1 |
| E434 | 21.1 | 93.4 | 2 | 277.5 | 99.5 | 1 |
| E435 | 25.3 | 86.3 | 2 | 169.8 | 99.6 | 1 |
| E436 | 29.5 | 84.4 | 2 | 377.0 | 99.8 | 1 |
| E437 | 59.8 | 92.1 | 2 | 1268.6 | 99.5 | 1 |
| E438 | 40.4 | 79.0 | 3 | <527.6 | 99.5 | 2 |
| E439 | 163.5 | 93.1 | 2 | 2395.9 | 100.5 | 1 |
| E440 | 22.1 | 97.0 | 2 | 309.2 | 102.4 | 1 |
| E441 | 46.7 | 93.2 | 2 | 2526.2 | 101.0 | 1 |
| E442 | 62.1 | 91.5 | 2 | 3066.1 | 99.1 | 1 |
| E443 | 40.8 | 92.3 | 2 | 242.8 | 99.1 | 1 |
| E444 | 168.9 | 93.1 | 2 | 774.1 | 99.9 | 1 |
| E445 | 85.4 | 77.6 | 2 | 2202.8 | 100.1 | 1 |
| E446 | 88.8 | 92.9 | 2 | 6251.2 | 99.3 | 3 |
| E447 | 101.9 | 79.4 | 2 | 3134.3 | 99.9 | 1 |
| E448 | 200.1 | 75.0 | 2 | 6005.7 | 99.9 | 1 |
| E449 | 287.7 | 86.8 | 2 | 39941.4 | 99.5 | 4 |
| E450 | 148.1 | 86.0 | 2 | 2518.4 | 99.1 | 2 |
| E451 | 293.2 | 84.6 | 2 | 19850.1 | 98.8 | 2 |
| E452 | 1073.7 | 81.5 | 2 | 5086.6 | 99.3 | 2 |
| E453 | 985.6 | 80.5 | 1 | 30483.6 | 97.3 | 2 |
| E454 | 2.1 | 98.5 | 2 | 68.5 | 100.0 | 1 |
| E455 | 38.2 | 93.4 | 2 | 272.8 | 101.6 | 1 |
| E456 | 14.8 | 98.0 | 2 | 96.8 | 101.4 | 1 |
| E457 | 339.2 | 91.0 | 2 | 3159.8 | 100.0 | 1 |
| E458 | 65.6 | 89.3 | 2 | 1038.0 | 102.7 | 1 |
| E459 | 10.7 | 97.9 | 2 | 125.3 | 100.9 | 1 |
| E460 | 3.6 | 94.7 | 4 | 47.5 | 102.8 | 1 |
| E461 | 42.2 | 93.1 | 3 | 268.5 | 100.6 | 1 |
| E462 | 93.7 | 87.8 | 2 | 2252.9 | 98.4 | 2 |
| E463 | 92.6 | 90.8 | 2 | 12125.8 | 99.1 | 2 |
| E464 | 68.7 | 86.6 | 2 | 1134.3 | 104.3 | 1 |
| E465 | 18.5 | 92.1 | 2 | 760.4 | 106.4 | 1 |
| E466 | 5.4 | 98.3 | 3 | 115.2 | 100.7 | 1 |
| E467 | 4.2 | 88.5 | 3 | 93.3 | 99.2 | 1 |
| E468 | 61.7 | 96.1 | 3 | 3926.2 | 99.3 | 2 |
| E469 | 149.5 | 89.8 | 2 | 4190.7 | 99.3 | 2 |
| E470 | 129.1 | 89.9 | 2 | 1556.8 | 102.6 | 1 |
| E471 | 15.2 | 94.2 | 3 | 237.2 | 106.7 | 1 |
| E472 | 9.8 | 96.1 | 3 | 284.4 | 105.9 | 1 |
| E473 | 42.6 | 89.9 | 2 | 771.9 | 107.0 | 1 |
| E474 | 15.8 | 92.6 | 2 | 424.8 | 100.2 | 1 |
| E475 | 13.5 | 97.5 | 2 | 521.5 | 100.5 | 1 |
| E476 | 14.7 | 98.2 | 2 | 318.3 | 100.5 | 1 |
| E477 | 38.7 | 94.4 | 2 | 223.7 | 99.4 | 1 |
| E478 | 25.7 | 96.8 | 2 | 834.0 | 100.0 | 1 |
| E479 | 14.9 | 94.2 | 2 | 1546.5 | 99.8 | 1 |
| E480 | 52.6 | 93.7 | 2 | 836.4 | 99.8 | 1 |
| E481 | 26.9 | 93.7 | 2 | 442.0 | 99.4 | 1 |
| E482 | 78.3 | 97.3 | 2 | 796.3 | 100.0 | 1 |
| E483 | 51.9 | 92.0 | 2 | 1769.8 | 99.2 | 1 |
| E484 | 55.0 | 92.8 | 2 | NT | NT | NT |
| E485 | 102.3 | 96.7 | 2 | 941.4 | 100.1 | 1 |
| E486 | 44.8 | 92.6 | 2 | 882.1 | 103.9 | 1 |
| E487 | 80.6 | 93.3 | 2 | 800.2 | 100.0 | 1 |
| E488 | 169.6 | 96.1 | 2 | 1822.5 | 99.9 | 1 |
| E489 | 119.0 | 93.5 | 2 | 1458.1 | 102.1 | 1 |
| E490 | 128.5 | 93.1 | 2 | 1608.9 | 99.2 | 1 |
| E491 | 111.5 | 93.1 | 2 | 666.3 | 99.7 | 1 |
| E492 | 227.0 | 90.5 | 2 | 8287.7 | 101.4 | 2 |
| E493 | 306.4 | 91.5 | 2 | 868.9 | 101.4 | 1 |
| E494 | 134.0 | 93.2 | 2 | 1645.8 | 101.2 | 1 |
| E495 | 586.0 | 90.1 | 2 | 18450.6 | 101.0 | 1 |
| E496 | 653.9 | 94.0 | 2 | 19212.4 | 100.2 | 1 |
| E497 | 829.1 | 96.6 | 1 | 1938.6 | 100.2 | 1 |
| E498 | 169.6 | 91.5 | 2 | 734.2 | 101.7 | 1 |
| E499 | 6.0 | 93.6 | 2 | 97.3 | 102.2 | 1 |
| E500 | 12.6 | 97.2 | 2 | 370.5 | 100.3 | 1 |
| E501 | 13.3 | 94.5 | 2 | 316.2 | 99.5 | 1 |
| E502 | 330.6 | 91.0 | 1 | 11167.9 | 98.8 | 3 |
| E503\hat{ } | 42.5 | 85.8 | 2 | 1947.1 | 99.1 | 4 |
| E504\hat{ } | 29.0 | 89.8 | 2 | <780.5 | 100.9 | 3 |
| E505 | 234.0 | 84.4 | 1 | 6124.9 | 99.8 | 2 |
| E506 | 92.0 | 91.5 | 2 | 4310.4 | 102.1 | 2 |
| E507 | 54.3 | 95.4 | 2 | 1458.5 | 102.8 | 2 |
| E508 | 36.6 | 88.7 | 2 | 383.7 | 106.2 | 1 |
| E509 | 12.0 | 87.6 | 2 | 1133.6 | 105.1 | 1 |
| E510 | 28.6 | 86.8 | 2 | 883.0 | 105.4 | 1 |
| E511 | 601.2 | 75.1 | 1 | 28640.3 | 101.2 | 2 |
| E512 | 51.8 | 89.3 | 1 | 1484.5 | 99.9 | 4 |
| E513 | 71.8 | 81.4 | 1 | 775.8 | 107.9 | 1 |
| E514 | 42.2 | 64.8 | 2 | 1970.4 | 99.9 | 1 |
| E515 | 56.5 | 76.7 | 2 | 7485.1 | 94.5 | 1 |
| E516 | 485.5 | 86.1 | 2 | 4767.4 | 103.7 | 1 |
| E517 | 81.3 | 90.5 | 2 | 2035.7 | 108.2 | 1 |
| E518 | 41.7 | 91.3 | 2 | 1028.0 | 101.4 | 1 |
| E519 | 17.6 | 95.3 | 2 | 353.9 | 106.9 | 1 |
| E520 | 1616.6 | 79.2 | 1 | 31488.9 | 98.7 | 1 |
| E521 | 531.6 | 87.9 | 1 | 9194.6 | 93.5 | 1 |
| E522 | 12.5 | 95.5 | 2 | 334.3 | 102.4 | 1 |
| E523 | 24.9 | 95.6 | 2 | 469.5 | 109.2 | 1 |
| E524 | 127.6 | 95.1 | 1 | 1057.6 | 106.0 | 1 |
| E525 | 74.2 | 92.6 | 2 | 299.7 | 104.2 | 1 |
| E526 | 375.7 | 88.0 | 1 | 11169.7 | 96.8 | 1 |
| E527 | 94.3 | 83.9 | 2 | 3808.3 | 100.8 | 1 |
| E528 | 14.2 | 89.1 | 3 | 477.3 | 101.4 | 1 |
| E529 | 107.6 | 85.8 | 1 | 5399.1 | 99.8 | 1 |
| E530 | 39.6 | 87.9 | 2 | 1836.0 | 100.2 | 1 |
| E531 | 97.9 | 86.9 | 2 | 3206.0 | 101.0 | 1 |
| E532 | 465.9 | 82.2 | 2 | 5547.7 | 98.6 | 1 |
| E533 | 31.2 | 87.1 | 2 | 838.8 | 99.5 | 1 |
| E534 | 191.6 | 83.5 | 2 | 7746.5 | 100.6 | 1 |
| E535 | 1587.3 | 74.0 | 1 | 130313.7 | 94.8 | 1 |
| E536 | 15.4 | 89.5 | 3 | 1360.1 | 101.0 | 1 |
| E537 | 31.8 | 79.1 | 2 | 1214.6 | 96.9 | 1 |
| E538 | 42.1 | 81.1 | 2 | 1805.4 | 101.1 | 1 |
| E539 | 58.0 | 83.4 | 2 | 3967.0 | 99.9 | 1 |
| E540 | 17.3 | 87.9 | 4 | 298.6 | 100.2 | 1 |
| E541 | 80.4 | 83.6 | 1 | 4590.3 | 99.0 | 1 |
| E542 | 167.4 | 82.7 | 1 | 5200.2 | 100.6 | 1 |
| E543 | 30.2 | 89.3 | 3 | 2594.8 | 99.0 | 1 |
| E544 | 12.4 | 90.9 | 3 | 670.2 | 100.3 | 1 |
| E545 | 11.4 | 90.8 | 4 | 410.6 | 99.1 | 1 |
| E546 | 7.8 | 92.2 | 3 | 50.3 | 100.6 | 1 |
| E547 | 11.5 | 91.7 | 3 | 135.1 | 100.6 | 1 |
| E548 | 15.6 | 87.0 | 2 | 103.5 | 98.9 | 1 |
| E549 | 11.8 | 89.0 | 3 | 475.9 | 99.4 | 1 |
| E550 | 13.9 | 90.7 | 3 | 533.7 | 101.0 | 1 |
| E551 | 44.3 | 88.9 | 2 | 1942.6 | 100.5 | 1 |
| E552 | 45.8 | 89.4 | 2 | 1145.7 | 100.8 | 1 |
| E553 | 49.2 | 87.9 | 2 | 1999.5 | 97.7 | 1 |
| E554 | 46.5 | 85.2 | 2 | 2744.7 | 95.3 | 1 |
| E555 | 134.0 | 88.0 | 1 | 750.2 | 100.9 | 1 |
| E556 | 260.4 | 76.9 | 1 | 7473.5 | 97.8 | 1 |
| E557 | 627.8 | 27.2 | 1 | >750000.0 | NT | 1 |
| E558\hat{ } | 761.5 | 69.6 | 1 | 91378.1 | 97.9 | 1 |
| E559 | 1132.5 | 86.6 | 1 | 6953.2 | 90.6 | 1 |
| E560 | 4.2 | 91.6 | 3 | 50.4 | 100.8 | 1 |
| E561 | 94.8 | 87.3 | 1 | 930.0 | 101.4 | 1 |
| E562 | 31.1 | 85.8 | 2 | 381.0 | 99.1 | 1 |
| E563 | 13.4 | 91.0 | 3 | 271.2 | 100.0 | 1 |
| E564 | 28.5 | 84.4 | 2 | 260.1 | 98.6 | 1 |
| E565\hat{ } | 36.9 | 87.3 | 2 | 339.2 | 99.3 | 1 |
| E566 | 77.5 | 84.5 | 2 | 463.2 | 99.4 | 1 |
| E567 | 16.9 | 89.7 | 3 | 422.4 | 102.0 | 1 |
| E568 | 59.8 | 87.0 | 2 | 1219.3 | 99.7 | 1 |
| E569 | 1645.3 | 74.3 | 1 | 3788.2 | 97.3 | 1 |
| E570 | 13.1 | 91.3 | 3 | 281.3 | 100.5 | 1 |
| E571 | 113.9 | 85.7 | 1 | 807.0 | 98.8 | 1 |
| E572 | 253.9 | 86.3 | 1 | 1993.0 | 102.9 | 1 |
| E573 | 22.2 | 91.2 | 3 | 150.4 | 99.6 | 1 |
| E574 | 227069.1 | 41.9 | 1 | >750000.0 | NT | 1 |
| E575 | 33.8 | 85.8 | 2 | 2248.1 | 98.5 | 1 |
| E576 | 27.4 | 88.1 | 2 | 1869.7 | 99.0 | 1 |
| E577 | 161.3 | 86.4 | 1 | 2198.2 | 107.9 | 1 |
| E578 | 26.8 | 87.8 | 2 | 1035.0 | 99.9 | 1 |
| E579 | 30.7 | 89.1 | 3 | 1175.7 | 98.9 | 1 |
| E580 | 38.7 | 82.7 | 2 | 1968.1 | 98.3 | 1 |
| E581 | 433.3 | 67.9 | 1 | 11508.4 | 92.3 | 1 |
| E582 | 20.9 | 82.8 | 2 | 564.3 | 98.4 | 1 |
| E583 | 72.1 | 82.3 | 1 | 1161.8 | 95.2 | 1 |
| E584 | 27.9 | 87.4 | 3 | 795.4 | 100.7 | 1 |
| E585 | 65.7 | 98.7 | 1 | 1358.1 | 100.4 | 1 |
| E586 | 12.4 | 86.5 | 2 | 290.7 | 100.4 | 1 |
| E587 | 189.5 | 73.0 | 1 | 5110.6 | 84.0 | 1 |
| E588 | 18.5 | 84.1 | 2 | 406.5 | 99.3 | 1 |
| E589 | 300.7 | 88.3 | 1 | 2347.1 | 98.4 | 1 |
| E590 | 10.3 | 87.2 | 2 | 549.0 | 100.0 | 1 |
| E591 | 6.1 | 91.6 | 3 | 176.5 | 100.0 | 1 |
| E592 | 13.5 | 90.2 | 3 | 226.7 | 101.6 | 1 |
| E593 | 22.1 | 88.4 | 2 | 432.3 | 100.8 | 1 |
| E594 | 32.3 | 85.2 | 2 | 1094.6 | 99.2 | 1 |
| E595 | 147.4 | 85.1 | 1 | 2250.6 | 103.1 | 1 |
| E596 | 1104.6 | 75.9 | 1 | 4860.4 | 101.5 | 1 |
| E597 | 427.1 | 84.3 | 1 | 6862.1 | 98.2 | 1 |
| E598 | 15.1 | 85.3 | 2 | 588.7 | 101.5 | 1 |
| E599 | 6.5 | 91.3 | 3 | 160.6 | 98.0 | 1 |
| E600 | 6.0 | 88.1 | 2 | 361.9 | 100.6 | 1 |
| E601 | 563.7 | 83.6 | 1 | 8240.0 | 96.8 | 1 |
| E602 | 2.1 | 93.9 | 4 | 75.5 | 98.9 | 1 |
| E603 | 7.5 | 91.0 | 3 | 185.9 | 99.7 | 1 |
| E604 | 4.7 | 88.3 | 3 | 113.8 | 99.6 | 1 |
| E605 | 779.6 | 85.2 | 1 | 3454.8 | 98.2 | 1 |
| E606 | 20.6 | 92.1 | 2 | 620.3 | 101.0 | 1 |
| E607 | 38.5 | 92.8 | 2 | 1130.5 | 97.7 | 1 |
| E608 | 53.0 | 92.7 | 2 | 945.0 | 100.5 | 1 |
| E609 | 19.9 | 91.1 | 2 | 1424.7 | 101.1 | 1 |
| E610 | 30.4 | 90.8 | 2 | 1116.9 | 100.8 | 1 |
| E611 | 8.4 | 92.7 | 2 | 225.4 | 100.7 | 1 |
| E612 | 29.5 | 91.9 | 2 | 910.5 | 99.4 | 1 |
| E613 | 19965.4 | 77.0 | 1 | >750000.0 | NT | 1 |
| E614 | 11640 | 75.1 | 1 | >750000.0 | NT | 1 |
| E615 | 5.2 | 89.5 | 3 | 36.3 | 100.6 | 1 |
| E616 | 3.1 | 90.0 | 3 | 136.5 | 100.2 | 1 |
| E617 | 6.4 | 87.3 | 3 | 142.6 | 99.4 | 1 |
| E618 | >500000.0 | NT | 1 | >750000.0 | NT | 1 |
| E619 | 49649.0 | 41.3 | 1 | >750000.0 | NT | 1 |
| E620 | 23.9 | 93.0 | 4 | 316.7 | 99.1 | 1 |
| E621 | 181.6 | 86.9 | 1 | 2205.7 | 98.6 | 1 |
| E622 | 1278.1 | 88.5 | 1 | 1553.3 | 101.0 | 1 |
| E623 | 29.6 | 91.9 | 2 | 6964.6 | 100.4 | 1 |
| E624 | 35.3 | 96.7 | 1 | 939.4 | 99.3 | 1 |
| E625 | 34.9 | 98.9 | 1 | 3830.7 | 105.8 | 1 |
| E626 | 92.8 | 92.1 | 2 | >30000000.0 | NT | 1 |
| E627 | 231.0 | 94.4 | 1 | 2610.8 | 98.9 | 1 |
| E628 | 283.8 | 94.9 | 1 | 41099.1 | 103.4 | 1 |
| E629 | 449.2 | 91.6 | 2 | 4858.3 | 101.7 | 1 |
| E630 | 489.3 | 62.7 | 2 | 65086.6 | 33.6 | 1 |
| E631 | 781.2 | 91.1 | 2 | 5669.5 | 100.7 | 1 |
| E632 | 839.5 | 97.7 | 1 | 332650.3 | 90.7 | 2 |
| E633 | 12.9 | 97.6 | 1 | NT | NT | NT |
| E634 | 80.9 | 88.2 | 2 | 2386.7 | 95.2 | 2 |
| E635 | 100.0 | 77.9 | 1 | NT | NT | NT |
| E636 | 117.8 | 97.4 | 1 | NT | NT | NT |
| E637 | 35.2 | 99.1 | 1 | 18726.9 | 102.1 | 1 |
| E638 | 307.9 | 62.8 | 1 | 2537.2 | 99.8 | 3 |
| E639 | 8.4 | 98.7 | 2 | 913.0 | 104.8 | 1 |
| E640 | 13.5 | 91.1 | 3 | 170.9 | 98.9 | 1 |
| E641 | 4657.4 | 84.4 | 1 | 68439.4 | 103.4 | 1 |
| E642 | 31031.3 | 78.5 | 1 | >750000.0 | NT | 1 |
| E643 | 9.2 | 92.5 | 2 | 424.5 | 100.7 | 1 |
| E644 | 11.6 | 91.8 | 2 | 516.3 | 100.7 | 1 |
| E645 | 98.1 | 85.1 | 1 | 1152.5 | 101.3 | 1 |
| E646 | 9.1 | 89.5 | 2 | 128.4 | 102.7 | 1 |
| E647 | 35.3 | 88.3 | 2 | 966.1 | 100.2 | 1 |
| E648 | 60.3 | 81.7 | 1 | 1801.2 | 100.9 | 1 |
| E649 | 181.8 | 78.6 | 1 | 2261.0 | 97.3 | 1 |
| E650 | 31.9 | 90.2 | 2 | 1416.9 | 101.1 | 1 |
| E651 | 8422.9 | 78.3 | 1 | 173025.3 | 97.7 | 1 |
| E652 | 194.8 | 84.4 | 1 | 956.4 | 98.1 | 1 |
| E653 | 9.3 | 92.9 | 2 | 24.7 | 103.7 | 1 |
| E654 | 65.3 | 84.2 | 1 | 236.8 | 104.0 | 1 |
| E655 | 1822.2 | 65.6 | 1 | >750000.0 | NT | 1 |
| E656 | 318.7 | 81.0 | 1 | 1957.8 | 100.2 | 1 |
| E657 | 480.6 | 82.2 | 1 | 7654.0 | 100.7 | 1 |
| E658 | 13099.2 | 84.9 | 1 | 107778.9 | 110.1 | 1 |
| E659 | 119.4 | 83.2 | 1 | 460.0 | 98.7 | 1 |
| E660 | 161.7 | 88.2 | 2 | 1057.9 | 97.7 | 1 |
| E661 | 23.6 | 91.9 | 2 | 317.7 | 100.4 | 1 |
| E662 | 3.5 | 98.1 | 2 | 94.9 | 101.2 | 1 |
| E663 | 7.0 | 98.0 | 2 | 72.1 | 103 | 1 |
| E664 | 518.6 | 96.5 | 1 | 5377.0 | 98.1 | 1 |
| E665 | 15.7 | 97.5 | 2 | 661.4 | 101.2 | 1 |
| E666 | 61.8 | 96.8 | 1 | 377.9 | 105.1 | 1 |
| E667 | 12.5 | 97.4 | 2 | 236.8 | 102.3 | 1 |
| E668 | 33.8 | 97.6 | 2 | 459.0 | 103.0 | 1 |
| E669 | 52.6 | 97.1 | 1 | 1311.5 | 101.5 | 1 |
| E670 | 31.2 | 97.0 | 2 | 193.6 | 101.3 | 1 |
| E671 | 78.7 | 96.2 | 1 | 3523.6 | 97.8 | 1 |
| E672 | 199.2 | 96.3 | 1 | 424.0 | 101.4 | 1 |
| E673 | 63.5 | 97.2 | 1 | 94.1 | 103.5 | 1 |
| E674 | 8.7 | 97.7 | 2 | 172.8 | 103.2 | 1 |
| E675 | 9878.1 | 95.6 | 1 | 2366.9 | 103.8 | 1 |
| E676 | 2604.1 | 96.1 | 1 | 746.4 | 105.7 | 1 |
| E677 | 1.2 | 98.4 | 2 | 19.2 | 103.0 | 1 |
| E678 | 134.3 | 98.0 | 1 | 2179.1 | 103.5 | 1 |
| E679 | 15.3 | 98.3 | 2 | 227.5 | 102.6 | 1 |
| E680 | 9.9 | 97.8 | 2 | 184.2 | 101.6 | 1 |
| E681 | 53.6 | 98.4 | 1 | 331.5 | 103.3 | 1 |
| E682 | 20.9 | 97.9 | 2 | 636.7 | 101.7 | 1 |
| E683 | 108.7 | 96.1 | 1 | 1359.1 | 103.0 | 1 |
| E684 | 176.3 | 98.6 | 1 | 6839.3 | 105.8 | 1 |
| E685 | 28.8 | 97.7 | 2 | 1085.7 | 101.0 | 1 |
| E686 | 515.1 | 87.3 | 1 | 8740.7 | 75.2 | 1 |
| E687 | 15.9 | 97.4 | 2 | 251.0 | 101.4 | 1 |
| E688 | 48.5 | 98.8 | 1 | 560.8 | 98.6 | 1 |
| E689 | 1101.4 | 94.0 | 1 | 836.4 | 101.3 | 1 |
| E690 | >500000.0 | NT | 1 | >750000.0 | NT | 1 |
| E691 | 399.6 | 61.3 | 1 | >750000.0 | NT | 1 |
| E692 | 5.8 | 98.4 | 2 | 427.9 | 100.7 | 1 |
| E693 | 6.4 | 97.9 | 1 | 50.2 | 99.5 | 1 |
| E694 | 4.7 | 97.6 | 1 | 41.1 | 103.1 | 1 |
| E695 | 5.2 | 98.1 | 1 | 70.6 | 103.6 | 1 |
| E696 | 32.9 | 96.3 | 1 | 92.7 | 103.1 | 1 |
| E697 | 11699.9 | 99.3 | 1 | 2135.0 | 99.9 | 1 |
| E698 | 2.1 | 97.0 | 1 | 189.6 | 101.7 | 1 |
| E699 | 7.3 | 97.4 | 1 | 128.1 | 102.2 | 1 |
| E700 | >500000.0 | NT | 1 | >750000.0 | NT | 1 |
| E701 | 8.7 | 96.8 | 1 | 37.6 | 102.7 | 1 |
| E702 | 35.0 | 97.7 | 1 | 338.5 | 100.4 | 1 |
| E703 | 7.0 | 97.9 | 1 | 97.5 | 102.2 | 1 |
| E704 | 207.3 | 96.5 | 1 | 12562.2 | 98.1 | 1 |
| E705 | 163.1 | 96.1 | 1 | 2298.5 | 100.4 | 1 |
| E706 | 32.8 | 98.8 | 2 | 700.1 | 103.9 | 1 |
| E707 | 26.2 | 94.5 | 2 | 731.3 | 101.3 | 1 |
| E708 | 7.2 | 98.5 | 2 | 617.9 | 101.7 | 1 |
| E709 | 108.0 | 95.6 | 1 | 341.4 | 100.8 | 1 |
| E710 | 17.9 | 97.2 | 1 | 2538.8 | 98.8 | 1 |
| E711 | 154.5 | 95.8 | 1 | 599.5 | 99.1 | 1 |
| E712 | 292.4 | 93.3 | 1 | 5464.9 | 107.3 | 1 |
| E713 | 568.0 | 97.2 | 1 | 705.1 | 99.8 | 1 |
| E714 | >500000.0 | NT | 1 | >750000.0 | NT | 1 |
| E715 | 734.0 | 74.7 | 1 | 5177.2 | 88.7 | 1 |
| E716 | 1157.8 | 74.6 | 1 | 11204.4 | 83.6 | 1 |
| E717 | 10.9 | 97.7 | 1 | 3152.9 | 101.4 | 1 |
| E718 | NT | NT | NT | 281.5 | 104.2 | 1 |
| E719 | NT | NT | NT | 5587.7 | 96.4 | 1 |
| E720 | 260.4 | 86.6 | 1 | 4645.5 | 101.1 | 1 |
| E721 | NT | NT | NT | 1893.8 | 102.8 | 1 |
| E722 | NT | NT | NT | 6910.3 | 98.3 | 1 |
| E723 | NT | NT | NT | 16431.1 | 105.8 | 1 |
| E724 | NT | NT | NT | 4228.2 | 98.8 | 1 |
| E725 | NT | NT | NT | 12551.6 | 102.2 | 1 |
| E726 | NT | NT | NT | NT | NT | NT |
| E727 | NT | NT | NT | 15303.2 | 96.8 | 1 |
| E728 | NT | NT | NT | 48037.8 | 103.2 | 1 |
| E729 | NT | NT | NT | 9374.8 | 100.4 | 1 |
| E730 | NT | NT | NT | 1483.9 | 83.2 | 1 |
| E731 | NT | NT | NT | 2571.5 | 98.4 | 1 |
| E732 | NT | NT | NT | 6373.4 | 95.3 | 1 |

### Prophetic Deuterated Analogs

General methods/reviews of obtaining metabolite profile and identifying metabolites of a compound are described in: Dalvie, et al., “Assessment of Three Human in Vitro Systems in the 5 Generation of Major Human Excretory and Circulating Metabolites,” Chemical Research in Toxicology, 2009, 22, 2, 357-368, tx8004357 (acs.org); King, R., “Biotransformations in Drug Metabolism,” Ch. 3, Drug Metabolism Handbook Introduction, https://doi.orq/10.1002/9781119851042.ch3; Wu, Y., et al, “Metabolite Identification in the Preclinical and Clinical Phase of Drug Development,” Current Drug Metabolism, 2021, 22, 11, 838-857, 10.2174/1389200222666211006104502; Godzien, J., et al, “Chapter Fifteen—Metabolite Annotation and Identification”.

Numerous publicly available and commercially available software tools are available to aid in the predictions of metabolic pathways and metabolites of compounds. Examples of such tools include, BioTransofrmer 3.0 (biotransformer.ca/new), which predicts the metabolic biotransformations of small molecules using a database of known metabolic reactions; MetaSite (moldiscovery.com/softwarelmetasite/), which predicts metabolic transformations related to cytochrome P450 and flavin-containing monooxygenase mediated reactions in phase I metabolism; and Lhasa Meteor Nexus (lhasalimited.orq/products/meteor-nexus.htm) offers prediction of metabolic pathways and metabolite structures using a range of machine learning models, which covers phase I and phase II biotransformations of small molecules.

Predicted deuterated analogs in the examples may afford certain therapeutic advantages resulting from greater metabolic stability, for example, increased in vivo half-life, reduced dosage requirements, increased STAT6 inhibition (competitive or time dependent), or an improvement in therapeutic index or tolerability.

A person with ordinary skill may make additional deuterated analogs of the disclosed compounds with different combinations of deuterium replacing hydrogen. Such additional deuterated analogs may provide similar therapeutic advantages that may be achieved by the deuterated analogs as illustrated below.

In the examples, the “Yᵢ” is used to indicate the order of the sites most likely to be metabolized based on MetaSite predictions for the respective compound, where i=1 indicates the most likely, i=2 indicates the second most likely site and so on. It is noted that where Yᵢ is indicated as being deuterated, D, each Yᵢ independently can be D as long as at least one Yᵢ is D. For example, if Yᵢ is deuterated and there are two Yᵢ groups in the compound, then one or both groups can be deuterated as long as at least one Yᵢ group is deuterated.

These examples are not intended to be limited to the specific compounds or locations of deuteration.

### Example D1

Formula D1 is the generic formula of deuterated Example 1, wherein Y₁, Y₂, Y₃, Y₄, Y₅, Y₆, Y₇, Y₈, Y₉, Y₁₀, and Y₁₁ are each independently H or D. Table 7 shows some examples of deuterated compounds based on the most likely metabolized sites that can be applied to the compounds predicted by MetaSite.

TABLE 7

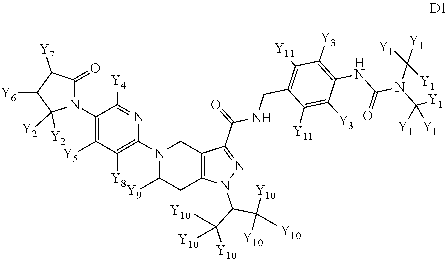

| Deuteration | | | Y₃ and/ | | | | | | | |
|---|---|---|---|---|---|---|---|---|---|---|
| Example | Y₁ | Y₂ | or Y₁₁ | Y₄ | Y₅ | Y₆ | Y₇ | Y₈ | Y₉ | Y₁₀ |
| D1A | D | H | H | H | H | H | H | H | H | H |
| D1B | D | D | H | H | H | H | H | H | H | H |
| D1C | D | D | D | H | H | H | H | H | H | H |
| D1D | D | H | D | H | H | H | H | H | H | H |
| D1E | D | H | H | D | H | H | H | H | H | H |
| D1F | D | H | H | H | D | H | H | H | H | H |
| D1G | D | H | H | H | H | D | H | H | H | H |
| D1H | D | H | H | H | H | H | D | H | H | H |
| D1I | D | H | H | H | H | H | H | D | H | H |
| D1J | D | H | H | H | H | H | H | H | D | H |
| D1K | D | H | H | H | H | H | H | H | H | D |
| D1L | D | D | D | D | H | H | H | H | H | H |
| D1M | H | D | D | H | H | H | H | H | H | H |
| D1N | H | D | H | D | H | H | H | H | H | H |
| D1O | H | D | H | H | D | H | H | H | H | H |
| D1P | H | D | H | H | H | D | H | H | H | H |
| D1Q | H | D | H | H | H | H | D | H | H | H |
| D1R | H | D | H | H | H | H | H | D | H | H |
| D1S | H | D | H | H | H | H | H | H | D | H |
| D1T | H | D | H | H | H | H | H | H | H | D |
| D1U | D | H | D | D | H | H | H | H | H | H |
| D1V | D | D | H | D | H | H | H | H | H | H |

### Example D2

Formula D2 is the generic formula of deuterated Example 2, wherein Y₁, Y₂, Y₃, Y₄, Y₅, Y₆, Y₇, Y₈, Y₉, and Y₁₀ are each independently H or D. Table 7 shows some examples of deuterated compounds based on the most likely metabolized sites that can be applied to the compounds predicted by MetaSite.

TABLE 7

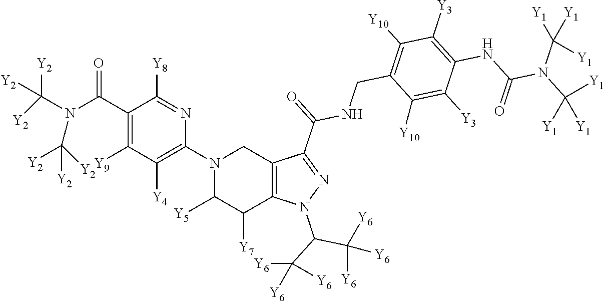

| Deuteration | | | | | | | | | | |
|---|---|---|---|---|---|---|---|---|---|---|
| Example | Y₁ | Y₂ | Y₃ | Y₄ | Y₅ | Y₆ | Y₇ | Y₈ | Y₉ | Y₁₀ |
| D2A | D | H | H | H | H | H | H | H | H | H |
| D2B | D | D | H | H | H | H | H | H | H | H |
| D2C | D | D | D | H | H | H | H | H | H | H |
| D2D | D | H | D | H | H | H | H | H | H | H |
| D2E | D | H | H | D | H | H | H | H | H | H |
| D2F | D | H | H | H | D | H | H | H | H | H |
| D2G | D | H | H | H | H | D | H | H | H | H |
| D2H | D | H | H | H | H | H | D | H | H | H |
| D2I | D | H | H | H | H | H | H | D | H | H |
| D2J | D | H | H | H | H | H | H | H | D | H |
| D2K | D | H | H | H | H | H | H | H | H | D |
| D2L | D | D | D | D | H | H | H | H | H | H |
| D2M | H | D | D | H | H | H | H | H | H | H |
| D2N | H | D | H | D | H | H | H | H | H | H |
| D2O | H | D | H | H | D | H | H | H | H | H |
| D2P | H | D | H | H | H | D | H | H | H | H |
| D2Q | H | D | H | H | H | H | D | H | H | H |
| D2R | H | D | H | H | H | H | H | D | H | H |
| D2S | H | D | H | H | H | H | H | H | D | H |
| D2T | H | D | H | H | H | H | H | H | H | D |
| D2U | D | H | D | D | H | H | H | H | H | H |
| D2V | D | D | H | D | H | H | H | H | H | H |

### Example D6

Formula D6 is the generic formula of deuterated Example 6, wherein Y₁, Y₂, Y₃, Y₄, Y₅, Y₆, Y₇, Y₈, Y₉, Y₁₀, and Y₁₁ are each independently H or D. Table 8 shows some examples of deuterated compounds based on the most likely metabolized sites that can be applied to the compounds predicted by MetaSite.

TABLE 8

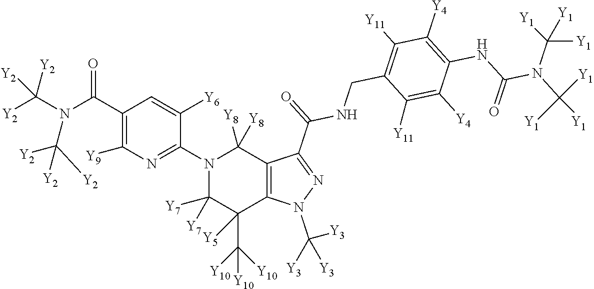

| Deuteration | | | | | | | | | | |
|---|---|---|---|---|---|---|---|---|---|---|
| Example | Y₁ | Y₂ | Y₃ and/or Y₁₁ | Y₄ | Y₅ | Y₆ | Y₇ | Y₈ | Y₉ | Y₁₀ |
| D6A | D | H | H | H | H | H | H | H | H | H |
| D6B | D | D | H | H | H | H | H | H | H | H |
| D6C | D | D | D | H | H | H | H | H | H | H |
| D6D | D | H | D | H | H | H | H | H | H | H |
| D6E | D | H | H | D | H | H | H | H | H | H |
| D6F | D | H | H | H | D | H | H | H | H | H |
| D6G | D | H | H | H | H | D | H | H | H | H |
| D6H | D | H | H | H | H | H | D | H | H | H |
| D6I | D | H | H | H | H | H | H | D | H | H |
| D6J | D | H | H | H | H | H | H | H | D | H |
| D6K | D | H | H | H | H | H | H | H | H | D |
| D6L | D | D | D | D | H | H | H | H | H | H |
| D6M | H | D | D | H | H | H | H | H | H | H |
| D6N | H | D | H | D | H | H | H | H | H | H |
| D6O | H | D | H | H | D | H | H | H | H | H |
| D6P | H | D | H | H | H | D | H | H | H | H |
| D6Q | H | D | H | H | H | H | D | H | H | H |
| D6R | H | D | H | H | H | H | H | D | H | H |
| D6S | H | D | H | H | H | H | H | H | D | H |
| D6T | H | D | H | H | H | H | H | H | H | D |
| D6U | D | H | D | D | H | H | H | H | H | H |
| D6V | D | D | H | D | H | H | H | H | H | H |

### Example D31

Formula D31 is the generic formula of deuterated Example 31, wherein Y₁, Y₂, Y₃, Y₄, Y₅, Y₆, Y₇, Y₈, Y₉, and Y₁₀ are each independently H or D. Table 9 shows some examples of deuterated compounds based on the most likely metabolized sites that can be applied to the compounds predicted by MetaSite.

TABLE 9

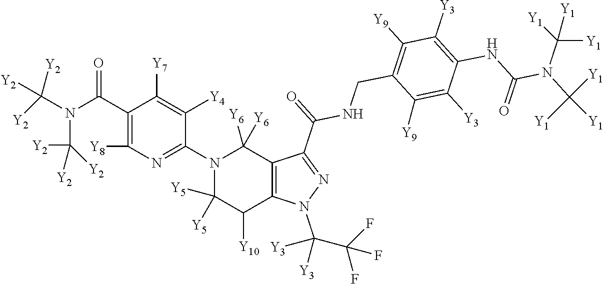

| Deuteration | | | | | | | | | | |
|---|---|---|---|---|---|---|---|---|---|---|
| Example | Y₁ | Y₂ | Y₃ | Y₄ | Y₅ | Y₆ | Y₇ | Y₈ | Y₉ | Y₁₀ |
| D31A | D | H | H | H | H | H | H | H | H | H |
| D31B | D | D | H | H | H | H | H | H | H | H |
| D31C | D | D | D | H | H | H | H | H | H | H |
| D31D | D | H | D | H | H | H | H | H | H | H |
| D31E | D | H | H | D | H | H | H | H | H | H |
| D31F | D | H | H | H | D | H | H | H | H | H |
| D31G | D | H | H | H | H | D | H | H | H | H |
| D31H | D | H | H | H | H | H | D | H | H | H |
| D31I | D | H | H | H | H | H | H | D | H | H |
| D31J | D | H | H | H | H | H | H | H | D | H |
| D31K | D | H | H | H | H | H | H | H | H | D |
| D31L | D | D | D | D | H | H | H | H | H | H |
| D31M | H | D | D | H | H | H | H | H | H | H |
| D31N | H | D | H | D | H | H | H | H | H | H |
| D31O | H | D | H | H | D | H | H | H | H | H |
| D31P | H | D | H | H | H | D | H | H | H | H |
| D31Q | H | D | H | H | H | H | D | H | H | H |
| D31R | H | D | H | H | H | H | H | D | H | H |
| D31S | H | D | H | H | H | H | H | H | D | H |
| D31T | H | D | H | H | H | H | H | H | H | D |
| D31U | D | H | D | D | H | H | H | H | H | H |
| D31V | D | D | H | D | H | H | H | H | H | H |

### Example D33

Formula D33 is the generic formula of deuterated Example 33, wherein Y₁, Y₂, Y₃, Y₄, Y₅, Y₆, Y₇, Y₈, Y₉, Y₁₀, and Y₁₁ are each independently H or D. Table 10 shows some examples of deuterated compounds based on the most likely metabolized sites that can be applied to the compounds predicted by MetaSite.

TABLE 10

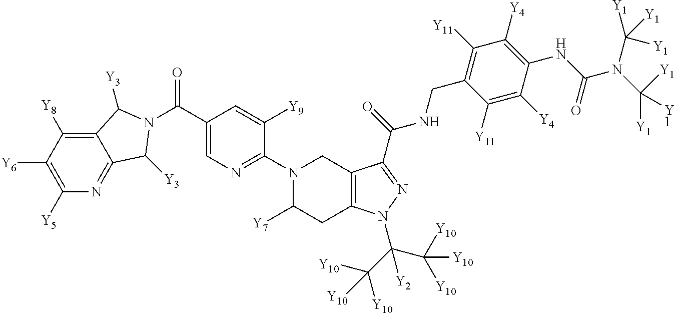

| Deuteration | | | | | | | | | | |
|---|---|---|---|---|---|---|---|---|---|---|
| Example | Y₁ | Y₂ | Y₃ | Y₄ and/or Y₁₁ | Y₅ | Y₆ | Y₇ | Y₈ | Y₉ | Y₁₀ |
| D33A | D | H | H | H | H | H | H | H | H | H |
| D33B | D | D | H | H | H | H | H | H | H | H |
| D33C | D | D | D | H | H | H | H | H | H | H |
| D33D | D | H | D | H | H | H | H | H | H | H |
| D33E | D | H | H | D | H | H | H | H | H | H |
| D33F | D | H | H | H | D | H | H | H | H | H |
| D33G | D | H | H | H | H | D | H | H | H | H |
| D33H | D | H | H | H | H | H | D | H | H | H |
| D33I | D | H | H | H | H | H | H | D | H | H |
| D33J | D | H | H | H | H | H | H | H | D | H |
| D33K | D | H | H | H | H | H | H | H | H | D |
| D33L | D | D | D | D | H | H | H | H | H | H |
| D33M | H | D | D | H | H | H | H | H | H | H |
| D33N | H | D | H | D | H | H | H | H | H | H |
| D33O | H | D | H | H | D | H | H | H | H | H |
| D33P | H | D | H | H | H | D | H | H | H | H |
| D33Q | H | D | H | H | H | H | D | H | H | H |
| D33R | H | D | H | H | H | H | H | D | H | H |
| D33S | H | D | H | H | H | H | H | H | D | H |
| D33T | H | D | H | H | H | H | H | H | H | D |
| D33U | D | H | D | D | H | H | H | H | H | H |
| D33V | D | D | H | D | H | H | H | H | H | H |

### Example D128

Formula D128 is the generic formula of deuterated Example 128, wherein Y₁, Y₂, Y₃, Y₄, Y₅, Y₆, Y₇, Y₈, Y₉, Y₁₀, and Y₁₁ are each independently H or D. Table 11 shows some examples of deuterated compounds based on the most likely metabolized sites that can be applied to the compounds predicted by MetaSite.

TABLE 11

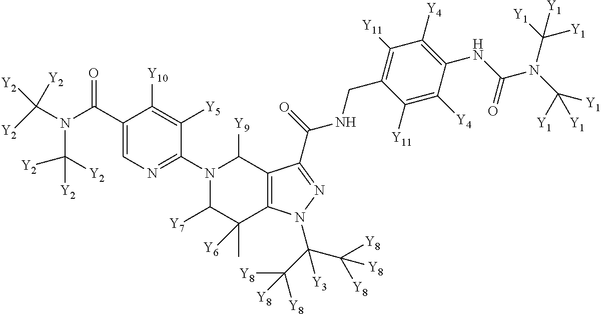

| Deuteration | | | | | | | | | | |
|---|---|---|---|---|---|---|---|---|---|---|
| Example | Y₁ | Y₂ | Y₃ | Y₄ and/or Y₁₁ | Y₅ | Y₆ | Y₇ | Y₈ | Y₉ | Y₁₀ |
| D128A | D | H | H | H | H | H | H | H | H | H |
| D128B | D | D | H | H | H | H | H | H | H | H |
| D128C | D | D | D | H | H | H | H | H | H | H |
| D128D | D | H | D | H | H | H | H | H | H | H |
| D128E | D | H | H | D | H | H | H | H | H | H |
| D128F | D | H | H | H | D | H | H | H | H | H |
| D128G | D | H | H | H | H | D | H | H | H | H |
| D128H | D | H | H | H | H | H | D | H | H | H |
| D128I | D | H | H | H | H | H | H | D | H | H |
| D128J | D | H | H | H | H | H | H | H | D | H |
| D128K | D | H | H | H | H | H | H | H | H | D |
| D128L | D | D | D | D | H | H | H | H | H | H |
| D128M | H | D | D | H | H | H | H | H | H | H |
| D128N | H | D | H | D | H | H | H | H | H | H |
| D128O | H | D | H | H | D | H | H | H | H | H |
| D128P | H | D | H | H | H | D | H | H | H | H |
| D128Q | H | D | H | H | H | H | D | H | H | H |
| D128R | H | D | H | H | H | H | H | D | H | H |
| D128S | H | D | H | H | H | H | H | H | D | H |
| D128T | H | D | H | H | H | H | H | H | H | D |
| D128U | D | H | D | D | H | H | H | H | H | H |
| D128V | D | D | H | D | H | H | H | H | H | H |

### Example D132

Formula D132 is the generic formula of deuterated Example 132, wherein Y₁, Y₂, Y₃, Y₄, Y₅, Y₆, Y₇, Y₈, Y₉, Y₁₀, and Y₁₁ are each independently H or D. Table 12 shows some examples of deuterated compounds based on the most likely metabolized sites that can be applied to the compounds predicted by MetaSite.

TABLE 12

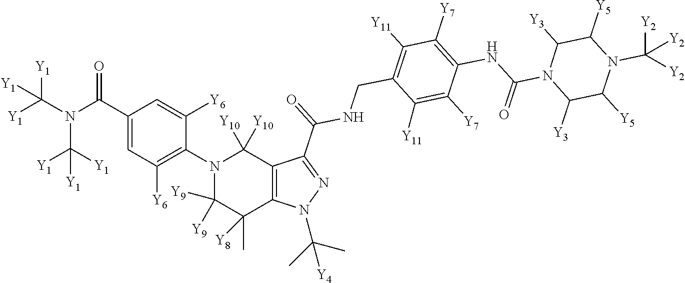

| Deuteration | | | | | | | | | | |
|---|---|---|---|---|---|---|---|---|---|---|
| Example | Y₁ | Y₂ | Y₃ | Y₄ | Y₅ | Y₆ | Y₇ and/or Y₁₁ | Y₈ | Y₉ | Y₁₀ |
| D132A | D | H | H | H | H | H | H | H | H | H |
| D132B | D | D | H | H | H | H | H | H | H | H |
| D132C | D | D | D | H | H | H | H | H | H | H |
| D132D | D | H | D | H | H | H | H | H | H | H |
| D132E | D | H | H | D | H | H | H | H | H | H |
| D132F | D | H | H | H | D | H | H | H | H | H |
| D132G | D | H | H | H | H | D | H | H | H | H |
| D132H | D | H | H | H | H | H | D | H | H | H |
| D132I | D | H | H | H | H | H | H | D | H | H |
| D132J | D | H | H | H | H | H | H | H | D | H |
| D132K | D | H | H | H | H | H | H | H | H | D |
| D132L | D | D | D | D | H | H | H | H | H | H |
| D132M | H | D | D | H | H | H | H | H | H | H |
| D132N | H | D | H | D | H | H | H | H | H | H |
| D132O | H | D | H | H | D | H | H | H | H | H |
| D132P | H | D | H | H | H | D | H | H | H | H |
| D132Q | H | D | H | H | H | H | D | H | H | H |
| D132R | H | D | H | H | H | H | H | D | H | H |
| D132S | H | D | H | H | H | H | H | H | D | H |
| D132T | H | D | H | H | H | H | H | H | H | D |
| D132U | D | H | D | D | H | H | H | H | H | H |
| D132V | D | D | H | D | H | H | H | H | H | H |

### Example DE215

Formula DE215 is the generic formula of deuterated Example E215, wherein Y₁, Y₂, Y₃, Y₄, Y₅, Y₆, Y₇, Y₈, Y₉, and Y₁₀ are each independently H or D. Table 13 shows some examples of deuterated compounds based on the most likely metabolized sites that can be applied to the compounds predicted by MetaSite.

TABLE 13

| | | | | | | | | | | DE215 |
|---|---|---|---|---|---|---|---|---|---|---|
| 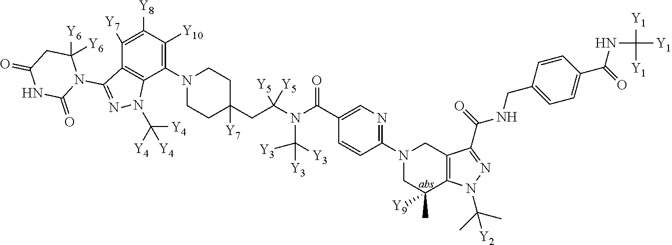 | | | | | | | | | | |
| Deuteration Example | Y₁ | Y₂ | Y₃ | Y₄ | Y₅ | Y₆ | Y₇ | Y₈ | Y₉ | Y₁₀ |
| DE215A | D | H | H | H | H | H | H | H | H | H |
| DE215B | D | D | H | H | H | H | H | H | H | H |
| DE215C | D | D | D | H | H | H | H | H | H | H |
| DE215D | D | H | D | H | H | H | H | H | H | H |
| DE215E | D | H | H | D | H | H | H | H | H | H |
| DE215F | D | H | H | H | D | H | H | H | H | H |
| DE215G | D | H | H | H | H | D | H | H | H | H |
| DE215H | D | H | H | H | H | H | D | H | H | H |
| DE215I | D | H | H | H | H | H | H | D | H | H |
| DE215J | D | H | H | H | H | H | H | H | D | H |
| DE215K | D | H | H | H | H | H | H | H | H | D |
| DE215L | D | D | D | D | H | H | H | H | H | H |
| DE215M | H | D | D | H | H | H | H | H | H | H |
| DE215N | H | D | H | D | H | H | H | H | H | H |
| DE215O | H | D | H | H | D | H | H | H | H | H |
| DE215P | H | D | H | H | H | D | H | H | H | H |
| DE215Q | H | D | H | H | H | H | D | H | H | H |
| DE215R | H | D | H | H | H | H | H | D | H | H |
| DE215S | H | D | H | H | H | H | H | H | D | H |
| DE215T | H | D | H | H | H | H | H | H | H | D |
| DE215U | D | H | D | D | H | H | H | H | H | H |
| DE215V | D | D | H | D | H | H | H | H | H | H |

### Example DE273

Formula DE273 is the generic formula of deuterated Example E273, wherein Y₁, Y₂, Y₃, Y₄, Y₅, Y₆, Y₇, Y₈, Y₉, and Y₁₀ are each independently H or D. Table 14 shows some examples of deuterated compounds based on the most likely metabolized sites that can be applied to the compounds predicted by MetaSite.

TABLE 14

| | | | | | | | | | | DE273 |
|---|---|---|---|---|---|---|---|---|---|---|
| 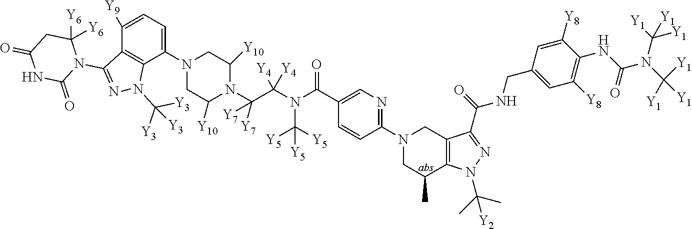 | | | | | | | | | | |
| Deuteration Example | Y₁ | Y₂ | Y₃ | Y₄ | Y₅ | Y₆ | Y₇ | Y₈ | Y₉ | Y₁₀ |
| DE273A | D | H | H | H | H | H | H | H | H | H |
| DE273B | D | D | H | H | H | H | H | H | H | H |
| DE273C | D | D | D | H | H | H | H | H | H | H |
| DE273D | D | H | D | H | H | H | H | H | H | H |
| DE273E | D | H | H | D | H | H | H | H | H | H |
| DE273F | D | H | H | H | D | H | H | H | H | H |
| DE273G | D | H | H | H | H | D | H | H | H | H |
| DE273H | D | H | H | H | H | H | D | H | H | H |
| DE273I | D | H | H | H | H | H | H | D | H | H |
| DE273J | D | H | H | H | H | H | H | H | D | H |
| DE273K | D | H | H | H | H | H | H | H | H | D |
| DE273L | D | D | D | D | H | H | H | H | H | H |
| DE273M | H | D | D | H | H | H | H | H | H | H |
| DE273N | H | D | H | D | H | H | H | H | H | H |
| DE273O | H | D | H | H | D | H | H | H | H | H |
| DE273P | H | D | H | H | H | D | H | H | H | H |
| DE273Q | H | D | H | H | H | H | D | H | H | H |
| DE273R | H | D | H | H | H | H | H | D | H | H |
| DE273S | H | D | H | H | H | H | H | H | D | H |
| DE273T | H | D | H | H | H | H | H | H | H | D |
| DE273U | D | H | D | D | H | H | H | H | H | H |
| DE273V | D | D | H | D | H | H | H | H | H | H |

### Example DE285

Formula DE285 is the generic formula of deuterated Example E285, wherein Y₁, Y₂, Y₃, Y₄, Y₅, Y₆, Y₇, Y₈, Y₉, and Y₁₀ are each independently H or D. Table 15 shows some examples of deuterated compounds based on the most likely metabolized sites that can be applied to the compounds predicted by MetaSite.

TABLE 15

| | | | | | | | | | | E285 |
|---|---|---|---|---|---|---|---|---|---|---|
| 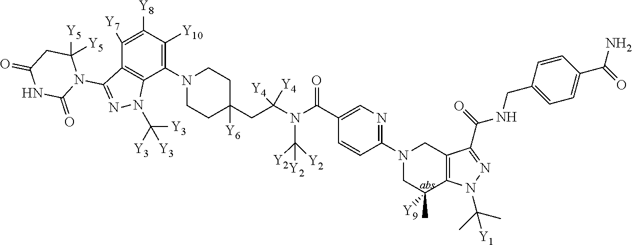 | | | | | | | | | | |
| Deuteration Example | Y₁ | Y₂ | Y₃ | Y₄ | Y₅ | Y₆ | Y₇ | Y₈ | Y₉ | Y₁₀ |
| DE285A | D | H | H | H | H | H | H | H | H | H |
| DE285B | D | D | H | H | H | H | H | H | H | H |
| DE285C | D | D | D | H | H | H | H | H | H | H |
| DE285D | D | H | D | H | H | H | H | H | H | H |
| DE285E | D | H | H | D | H | H | H | H | H | H |
| DE285F | D | H | H | H | D | H | H | H | H | H |
| DE285G | D | H | H | H | H | D | H | H | H | H |
| DE285H | D | H | H | H | H | H | D | H | H | H |
| DE285I | D | H | H | H | H | H | H | D | H | H |
| DE285J | D | H | H | H | H | H | H | H | D | H |
| DE285K | D | H | H | H | H | H | H | H | H | D |
| DE285L | D | D | D | D | H | H | H | H | H | H |
| DE285M | H | D | D | H | H | H | H | H | H | H |
| DE285N | H | D | H | D | H | H | H | H | H | H |
| DE285O | H | D | H | H | D | H | H | H | H | H |
| DE285P | H | D | H | H | H | D | H | H | H | H |
| DE285Q | H | D | H | H | H | H | D | H | H | H |
| DE285R | H | D | H | H | H | H | H | D | H | H |
| DE285S | H | D | H | H | H | H | H | H | D | H |
| DE285T | H | D | H | H | H | H | H | H | H | D |
| DE285U | D | H | D | D | H | H | H | H | H | H |
| DE285V | D | D | H | D | H | H | H | H | H | H |

### Example DE545

Formula DE545 is the generic formula of deuterated Example E545, wherein Y₁, Y₂, Y₃, Y₄, Y₅, Y₆, Y₇, Y₈, Y₉, and Y₁₀ are each independently H or D. Table 16 shows some examples of deuterated compounds based on the most likely metabolized sites that can be applied to the compounds predicted by MetaSite.

TABLE 16

| | | | | | | | | | | DE545 |
|---|---|---|---|---|---|---|---|---|---|---|
| 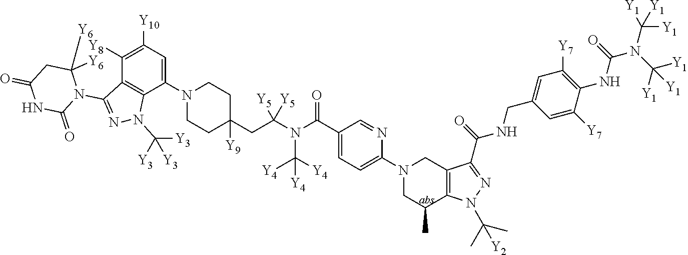 | | | | | | | | | | |
| Deuteration Example | Y₁ | Y₂ | Y₃ | Y₄ | Y₅ | Y₆ | Y₇ | Y₈ | Y₉ | Y₁₀ |
| DE545A | D | H | H | H | H | H | H | H | H | H |
| DE545B | D | D | H | H | H | H | H | H | H | H |
| DE545C | D | D | D | H | H | H | H | H | H | H |
| DE545D | D | H | D | H | H | H | H | H | H | H |
| DE545E | D | H | H | D | H | H | H | H | H | H |
| DE545F | D | H | H | H | D | H | H | H | H | H |
| DE545G | D | H | H | H | H | D | H | H | H | H |
| DE545H | D | H | H | H | H | H | D | H | H | H |
| DE545I | D | H | H | H | H | H | H | D | H | H |
| DE545J | D | H | H | H | H | H | H | H | D | H |
| DE545K | D | H | H | H | H | H | H | H | H | D |
| DE545L | D | D | D | D | H | H | H | H | H | H |
| DE545M | H | D | D | H | H | H | H | H | H | H |
| DE545N | H | D | H | D | H | H | H | H | H | H |
| DE545O | H | D | H | H | D | H | H | H | H | H |
| DE545P | H | D | H | H | H | D | H | H | H | H |
| DE545Q | H | D | H | H | H | H | D | H | H | H |
| DE545R | H | D | H | H | H | H | H | D | H | H |
| DE545S | H | D | H | H | H | H | H | H | D | H |
| DE545T | H | D | H | H | H | H | H | H | H | D |
| DE545U | D | H | D | D | H | H | H | H | H | H |
| DE545V | D | D | H | D | H | H | H | H | H | H |

### Example DE602

Formula DE602 is the generic formula of deuterated Example E602, wherein Y₁, Y₂, Y₃, Y₄, Y₅, Y₆, Y₇, Y₈, Y₉, and Y₁₀ are each independently H or D. Table 17 shows some examples of deuterated compounds based on the most likely metabolized sites that can be applied to the compounds predicted by MetaSite.

TABLE 17

| | | | | | | | | | | DE602 |
|---|---|---|---|---|---|---|---|---|---|---|
| 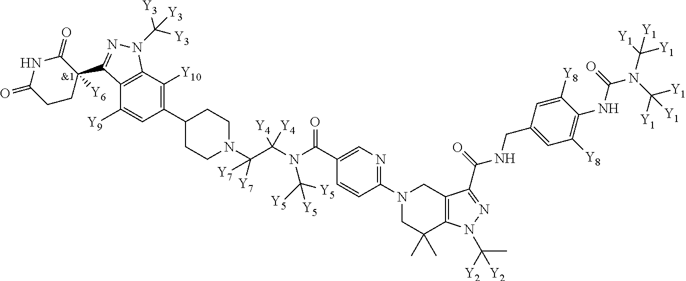 | | | | | | | | | | |
| Deuteration Example | Y₁ | Y₂ | Y₃ | Y₄ | Y₅ | Y₆ | Y₇ | Y₈ | Y₉ | Y₁₀ |
| DE602A | D | H | H | H | H | H | H | H | H | H |
| DE602B | D | D | H | H | H | H | H | H | H | H |
| DE602C | D | D | D | H | H | H | H | H | H | H |
| DE602D | D | H | D | H | H | H | H | H | H | H |
| DE602E | D | H | H | D | H | H | H | H | H | H |
| DE602F | D | H | H | H | D | H | H | H | H | H |
| DE602G | D | H | H | H | H | D | H | H | H | H |
| DE602H | D | H | H | H | H | H | D | H | H | H |
| DE602I | D | H | H | H | H | H | H | D | H | H |
| DE602J | D | H | H | H | H | H | H | H | D | H |
| DE602K | D | H | H | H | H | H | H | H | H | D |
| DE602L | D | D | D | D | H | H | H | H | H | H |
| DE602M | H | D | D | H | H | H | H | H | H | H |
| DE602N | H | D | H | D | H | H | H | H | H | H |
| DE602O | H | D | H | H | D | H | H | H | H | H |
| DE602P | H | D | H | H | H | D | H | H | H | H |
| DE602Q | H | D | H | H | H | H | D | H | H | H |
| DE602R | H | D | H | H | H | H | H | D | H | H |
| DE602S | H | D | H | H | H | H | H | H | D | H |
| DE602T | H | D | H | H | H | H | H | H | H | D |
| DE602U | D | H | D | D | H | H | H | H | H | H |
| DE602V | D | D | H | D | H | H | H | H | H | H |

### Example DE620

Formula DE620 is the generic formula of deuterated Example E620, wherein Y₁, Y₂, Y₃, Y₄, Y₅, Y₆, Y₇, Y₈, Y₉, and Y₁₀ are each independently H or D. Table 18 shows some examples of deuterated compounds based on the most likely metabolized sites that can be applied to the compounds predicted by MetaSite.

TABLE 18

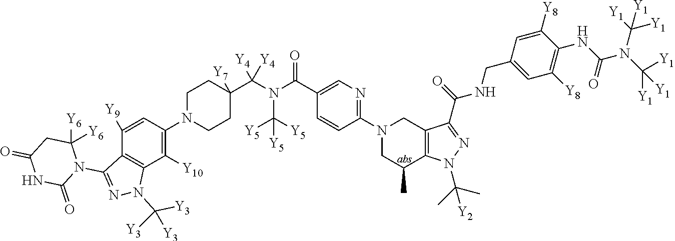

| Deuteration Example | Y₁ | Y₂ | Y₃ | Y₄ | Y₅ | Y₆ | Y₇ | Y₈ | Y₉ | Y₁₀ |
|---|---|---|---|---|---|---|---|---|---|---|
| DE620A | D | H | H | H | H | H | H | H | H | H |
| DE620B | D | D | H | H | H | H | H | H | H | H |
| DE620C | D | D | D | H | H | H | H | H | H | H |
| DE620D | D | H | D | H | H | H | H | H | H | H |
| DE620E | D | H | H | D | H | H | H | H | H | H |
| DE620F | D | H | H | H | D | H | H | H | H | H |
| DE620G | D | H | H | H | H | D | H | H | H | H |
| DE620H | D | H | H | H | H | H | D | H | H | H |
| DE620I | D | H | H | H | H | H | H | D | H | H |
| DE620J | D | H | H | H | H | H | H | H | D | H |
| DE620K | D | H | H | H | H | H | H | H | H | D |
| DE620L | D | D | D | D | H | H | H | H | H | H |
| DE620M | H | D | D | H | H | H | H | H | H | H |
| DE620N | H | D | H | D | H | H | H | H | H | H |
| DE620O | H | D | H | H | D | H | H | H | H | H |
| DE620P | H | D | H | H | H | D | H | H | H | H |
| DE620Q | H | D | H | H | H | H | D | H | H | H |
| DE620R | H | D | H | H | H | H | H | D | H | H |
| DE620S | H | D | H | H | H | H | H | H | D | H |
| DE620T | H | D | H | H | H | H | H | H | H | D |
| DE620U | D | H | D | D | H | H | H | H | H | H |
| DE620V | D | D | H | D | H | H | H | H | H | H |

### Example DE643

Formula DE643 is the generic formula of deuterated Example E643, wherein Y₁, Y₂, Y₃, Y₄, Y₅, Y₆, Y₇, Y₈, Y₉, and Y₁₀ are each independently H or D. Table 19 shows some examples of deuterated compounds based on the most likely metabolized sites that can be applied to the compounds predicted by MetaSite.

TABLE 19

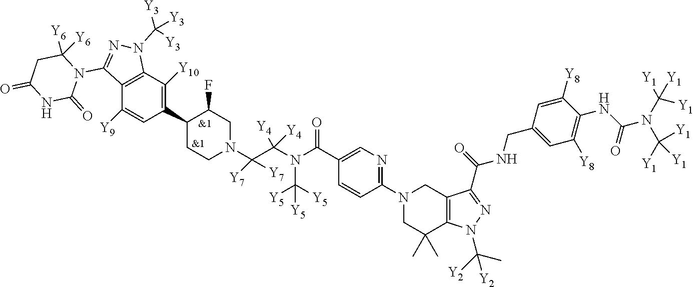

| Deuteration Example | Y₁ | Y₂ | Y₃ | Y₄ | Y₅ | Y₆ | Y₇ | Y₈ | Y₉ | Y₁₀ |
|---|---|---|---|---|---|---|---|---|---|---|
| DE643A | D | H | H | H | H | H | H | H | H | H |
| DE643B | D | D | H | H | H | H | H | H | H | H |
| DE643C | D | D | D | H | H | H | H | H | H | H |
| DE643D | D | H | D | H | H | H | H | H | H | H |
| DE643E | D | H | H | D | H | H | H | H | H | H |
| DE643F | D | H | H | H | D | H | H | H | H | H |
| DE643G | D | H | H | H | H | D | H | H | H | H |
| DE643H | D | H | H | H | H | H | D | H | H | H |
| DE643I | D | H | H | H | H | H | H | D | H | H |
| DE643J | D | H | H | H | H | H | H | H | D | H |
| DE643K | D | H | H | H | H | H | H | H | H | D |
| DE643L | D | D | D | D | H | H | H | H | H | H |
| DE643M | H | D | D | H | H | H | H | H | H | H |
| DE643N | H | D | H | D | H | H | H | H | H | H |
| DE643O | H | D | H | H | D | H | H | H | H | H |
| DE643P | H | D | H | H | H | D | H | H | H | H |
| DE643Q | H | D | H | H | H | H | D | H | H | H |
| DE643R | H | D | H | H | H | H | H | D | H | H |
| DE643S | H | D | H | H | H | H | H | H | D | H |
| DE643T | H | D | H | H | H | H | H | H | H | D |
| DE643U | D | H | D | D | H | H | H | H | H | H |
| DE643V | D | D | H | D | H | H | H | H | H | H |

### Example DE685

Formula DE685 is the generic formula of deuterated Example E685, wherein Y₁, Y₂, Y₃, Y4, Y₅, Y₆, Y₇, Y₈, Y₉, and Y₁₀ are each independently H or D. Table 20 shows some examples of deuterated compounds based on the most likely metabolized sites that can be applied to the compounds predicted by MetaSite.

TABLE 20

| | | | | | | | | | | DE619 |
|---|---|---|---|---|---|---|---|---|---|---|
| 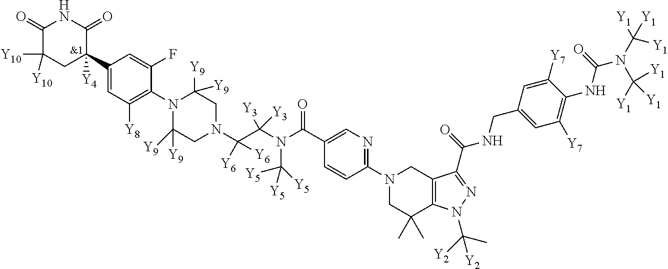 | | | | | | | | | | |
| Deuteration Example | Y₁ | Y₂ | Y₃ | Y₄ | Y₅ | Y₆ | Y₇ | Y₈ | Y₉ | Y₁₀ |
| DE685A | D | H | H | H | H | H | H | H | H | H |
| DE685B | D | D | H | H | H | H | H | H | H | H |
| DE685C | D | D | D | H | H | H | H | H | H | H |
| DE685D | D | H | D | H | H | H | H | H | H | H |
| DE685E | D | H | H | D | H | H | H | H | H | H |
| DE685F | D | H | H | H | D | H | H | H | H | H |
| DE685G | D | H | H | H | H | D | H | H | H | H |
| DE685H | D | H | H | H | H | H | D | H | H | H |
| DE685I | D | H | H | H | H | H | H | D | H | H |
| DE685J | D | H | H | H | H | H | H | H | D | H |
| DE685K | D | H | H | H | H | H | H | H | H | D |
| DE685L | D | D | D | D | H | H | H | H | H | H |
| DE685M | H | D | D | H | H | H | H | H | H | H |
| DE685N | H | D | H | D | H | H | H | H | H | H |
| DE685O | H | D | H | H | D | H | H | H | H | H |
| DE685P | H | D | H | H | H | D | H | H | H | H |
| DE685Q | H | D | H | H | H | H | D | H | H | H |
| DE685R | H | D | H | H | H | H | H | D | H | H |
| DE685S | H | D | H | H | H | H | H | H | D | H |
| DE685T | H | D | H | H | H | H | H | H | H | D |
| DE685U | D | H | D | D | H | H | H | H | H | H |
| DE685V | D | D | H | D | H | H | H | H | H | H |

### Example DE710

Formula DE710 is the generic formula of deuterated Example E710, wherein Y₁, Y₂, Y₃, Y₄, Y₅, Y₆, Y₇, Y₈, Y₉, and Y₁₀ are each independently H or D. Table 21 shows some examples of deuterated compounds based on the most likely metabolized sites that can be applied to the compounds predicted by MetaSite.

TABLE 21

| | | | | | | | | | | DE710 |
|---|---|---|---|---|---|---|---|---|---|---|
| 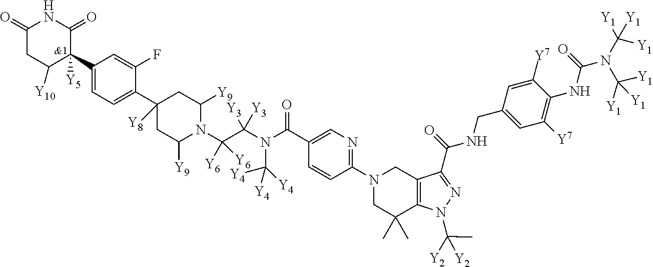 | | | | | | | | | | |
| Deuteration Example | Y₁ | Y₂ | Y₃ | Y₄ | Y₅ | Y₆ | Y₇ | Y₈ | Y₉ | Y₁₀ |
| DE710A | D | H | H | H | H | H | H | H | H | H |
| DE710B | D | D | H | H | H | H | H | H | H | H |
| DE710C | D | D | D | H | H | H | H | H | H | H |
| DE710D | D | H | D | H | H | H | H | H | H | H |
| DE710E | D | H | H | D | H | H | H | H | H | H |
| DE710F | D | H | H | H | D | H | H | H | H | H |
| DE710G | D | H | H | H | H | D | H | H | H | H |
| DE710H | D | H | H | H | H | H | D | H | H | H |
| DE710I | D | H | H | H | H | H | H | D | H | H |
| DE710J | D | H | H | H | H | H | H | H | D | H |
| DE710K | D | H | H | H | H | H | H | H | H | D |
| DE710L | D | D | D | D | H | H | H | H | H | H |
| DE710M | H | D | D | H | H | H | H | H | H | H |
| DE710N | H | D | H | D | H | H | H | H | H | H |
| DE710O | H | D | H | H | D | H | H | H | H | H |
| DE710P | H | D | H | H | H | D | H | H | H | H |
| DE710Q | H | D | H | H | H | H | D | H | H | H |
| DE710R | H | D | H | H | H | H | H | D | H | H |
| DE710S | H | D | H | H | H | H | H | H | D | H |
| DE710T | H | D | H | H | H | H | H | H | H | D |
| DE710U | D | H | D | D | H | H | H | H | H | H |
| DE710V | D | D | H | D | H | H | H | H | H | H |

It will be apparent to those skilled in the art that various modifications and variations may be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

LENGTHY TABLES
The patent application contains a lengthy table section. A copy of the table is available in electronic form from the USPTO web site (https://seqdata.uspto.gov/docdetail?docId=US20260345786A1). An electronic copy of the table will also be available from the USPTO upon request and payment of the fee set forth in 37 CFR 1.19(b)(3).

1. A compound of Formula I, or a pharmaceutically acceptable salt thereof:
M-T-Z I
wherein Z is a ubiquitin ligase binding moiety;
wherein T is a linker that connects Z to M;
wherein M is a targeting moiety having the Formula M: 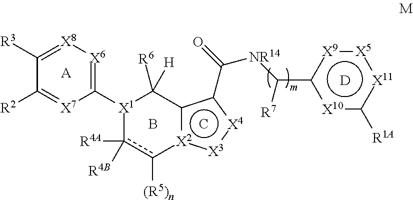
wherein
X¹ is CH, CNH₂, or N;
X² is C or N;
X³ is CR¹³, N, NR¹³, O, or S;
X⁴ is CH, N, or NR¹³;
X⁵ is CR^{1B} or N;
X⁶ is CH, CR¹⁵, or N;
X⁷ is CH, N, R^{2A}, or CF;
X⁸ is CH or N;
X⁹ is CR^{1B} or N;
X¹⁰ is CR^{1B} or N;
X¹¹ is CRI, N, or NCH₃;
R¹ is —NR¹⁰—C(═O)—, —NR¹⁰—C(═O)—NR¹⁰—, H, —C(═O)—NR¹⁰—, —NHR⁸, —OH, —C₁₋₄ hydroxyalkyl, —C₁₋₃ fluoroalkyl, —C₀₋₁ alkylene-C₂₋₆ cyclyl, —C(═O)—NR¹⁰R⁸, —C(═O)—NR¹⁰—C₀₋₁ alkylene-phenyl, —C₀₋₁ alkylene-C₁₋₅ heterocyclyl, —O-C₁₋₅ heterocyclyl, or —C₁₋₅ alkyl; wherein the —C₁₋₅ heterocyclyl or the —C₂₋₆ cyclyl of R¹ is optionally substituted with one, two, or three of —C₁₋₃ alkyl, halogen, —OH, —C(═O)—N(R¹⁰)₂, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₅ alkyl of R¹ is optionally substituted with one or two of oxo, —NR^{10A}R¹¹, or —NR¹⁰R¹¹, a 5 to 6 membered cycloalkyl, a 5 to 6 membered heterocycloalkyl, cyano, fluoromethoxy, or methoxy; wherein the respective a 5 to 6 membered cycloalkyl and a 5 to 6 membered heterocycloalkyl groups of R¹ are optionally substituted with one or two of —OH, oxo, halogen, —C₁₋₃ alkoxy, —C₁₋₃ alkyl-C₁₋₃ alkoxy, or —C₁₋₃ alkyl;
R^{1A} is H, halogen, —C₂₋₃ heterocyclyl, oxo, —C(═O)NR¹⁰R¹¹; or —CH₃; wherein the —C₂₋₃ heterocyclyl is optionally substituted with —C₁₋₃ alkyl; or
R¹ and R^{1A} taken together form a C₄₋₇ heterocycloalkyl fused to Ring D or a C₄₋₇ heteroaryl fused to Ring D; wherein the C₄₋₇ heterocycloalkyl or the C₄₋₇ heteroaryl is optionally substituted with one or two oxo or —C₂₋₃ oxoalkyl;
each R^{1B} independently is H, —CH₃, F, Cl, or methoxy;
R² is H, —C₁₋₃ alkyl, —C₁₋₃ alkoxy, —C₁₋₃ fluoroalkyl, or halogen; or
R² and R^{2A} taken together form a six membered heteroaryl fused to Ring A;
R³ is —C(═O)—, —C(═O)—NR^{12A}—, —NR^{12A}—C(═O)—, —C₂₋₇ heterocyclylene-, —C₁₋₃ alkyl, —C₁₋₁₀ heterocyclyl, —P(═O)(CH₃)₂, —S(═O)CH₃, —NH—S(═O)₂CH₃, or —NH—C(═O)—C₁₋₃ alkyl; wherein the —C₁₋₃ alkyl of R³ is optionally substituted with one, two, or three of oxo, halogen, —C₃₋₇ cycloalkyl, —OH, —NR¹²R^{12A}, or cyano; wherein the —C₁₋₁₀ heterocyclyl is optionally substituted with one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo; or
R² and R³ taken together form a C₃₋₆ heterocyclyl fused to Ring A optionally substituted with one, two, or three of —C₁₋₃ alkyl, —C₂₋₃ oxoalkyl, or oxo;
R^{4A} is H, —OH, —C₁₋₃ fluoroalkyl, or —C₁₋₃ alkyl;
R^{4B} is H or is absent; or
R^{4A} and R^{4B} taken together form cyclopropyl;
each R⁵ independently is H, halogen, —OH, cyclopropyl, —C₁₋₃ fluoroalkyl, or —C₁₋₃ alkyl;
or two R⁵ groups taken together form a C₃₋₄ cycloalkyl or a C₃₋₅ heterocycloalkyl;
R⁶ is H, —OH, cyano, or —C₁₋₃ alkyl; or
R^{4A} and R⁶ or one R⁵ and R⁶ taken together form a C₁₋₃ alkyl bridge or a C₁₋₃ heteroalkyl bridge; or
R^{4B} and one R⁵ taken together form a C₃₋₅ cycloalkyl fused to Ring B;
R⁷ is H, —C₁₋₃ alkyl, or —C₁₋₃ hydroxyalkyl; or
R⁸ is H, —C₁₋₃ alkyl, —SO₂CH₃, or a —C₂₋₅ heterocyclyl; wherein the —C₁₋₃ alkyl of R⁸ is optionally substituted with one, two, or three of oxo, —C₃₋₉ heterocyclyl, —C₁₋₃ alkoxy, cyanoimine, —C₅₋₆ aryl, —O—C₅₋₆ aryl, or —NR⁹R¹⁰; wherein the —C₂₋₅ heterocyclyl of R⁸ is optionally substituted with one, two, or three of oxo, halogen, —C₀₋₁ alkylene-NR¹⁰R¹¹, —OH, —C₁₋₃ hydroxyalkyl, C₁₋₃ alkoxy optionally substituted with —NR¹⁰R¹¹, —C₁₋₃ alkyl-C₁₋₃ alkoxy, —C₁₋₃ oxoalkyl, or —C₁₋₃ alkyl; wherein the —C₃₋₉ heterocycloalkyl of R⁸ is optionally substituted with one, two, or three of —C₁₋₃ alkyl, —OH, —C₁₋₃ hydroxyalkyl, —O-C₁₋₃ hydroxyalkyl, —C₁₋₃ alkoxy, —C₁₋₃ alkyl-C₁-3 alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O—C₃₋₅ heterocycloalkyl, phenyl, or —C₀₋₁ alkylene-NR¹⁰R¹¹; wherein the phenyl is optionally substituted with halogen; wherein the —C₅₋₆ aryl is optionally substituted with a C₄₋₅ heterocycloalkyl or halogen;
each R⁹ independently is H, —C₁₋₅ alkyl, —C₁₋₄ hydroxyalkyl, —C₁₋₃ fluoroalkyl, —C₃₋₅ cycloalkyl, —C₃₋₅ heterocycloalkyl, —methylene-phenylene-NH—C(═O)—NR¹⁰R¹¹, or —C₃₋₅ heterocycloalkyl; wherein the —C₁₋₅ alkyl of R⁹ is optionally substituted with one, two, or three of —NR¹⁰R¹¹, —C₃₋₅ cycloalkyl, —C₃₋₅ heterocycloalkyl, cyano, fluoromethoxy, or methoxy; wherein the respective —C₃₋₅ cycloalkyl and —C₃₋₅ heterocycloalkyl groups of R⁹ are optionally substituted with one or two of —OH, oxo, halogen, —C₁₋₃ alkoxy, —C₁₋₃ alkyl-C₁₋₃ alkoxy, or —C₁₋₃ alkyl;
each R¹⁰ independently is H or —C₁₋₄ alkyl; or
each R^{10A} independently is H, —C₁₋₅ alkyl, —C₁₋₄ hydroxyalkyl, —C₁₋₃ fluoroalkyl, —C₃₋₅ cycloalkyl, —C₃₋₅ heterocyclyl, —methylene-phenylene-NH—C(═O)—NR¹⁰R¹¹, or —C₀₋₁ alkylene-C₃₋₅ heterocycloalkyl optionally substituted with —C₁₋₃ alkyl; wherein the —C₁₋₅ alkyl of R^{10A} is optionally substituted with one, two, or three of —NR¹⁰R¹¹, —C₃₋₅ cycloalkyl, —C₃₋₅ heterocycloalkyl, cyano, fluoromethoxy, or methoxy; wherein the respective —C₃₋₅ cycloalkyl and —C₃₋₅ heterocycloalkyl groups of R^{10A} are optionally substituted with one or two of —OH, oxo, halogen, —C₁₋₃ alkoxy, —C₁₋₃ alkyl-C₁₋₃ alkoxy, or —C₁₋₃ alkyl;
each R¹¹ independently is H or —C₁₋₃ alkyl;
R¹² is H, —C₁₋₃ alkyl, —C₁₋₃ hydroxyalkyl, —(CH₂)₀₋₂ alkylene-C₃₋₅ heterocycloalkyl, —(CH₂)₁₋₂ alkylene-O—C₃₋₅ heterocycloalkyl, —methylene-C(═O)—NR¹⁰R¹¹, or -methylene-phenylene-NH—C(═O)—NR¹⁰R¹¹; wherein the —(CH₂)₀₋₂ alkylene-C₃₋₅ heterocycloalkyl or the —(CH₂)₁₋₂ alkylene-O—C₃₋₅ heterocycloalkyl of R¹² is optionally substituted with —C₁₋₄ alkyl, —C₁₋₃ alkyl-C₁₋₃ alkoxy, —(CH₂)₀₋₁ alkylene-phenyl, halogen, —C₁₋₃ fluoroalkyl, or —(CH₂)₀₋₁ alkylene-C₃₋₅ heterocycloalkyl; wherein the —C₁₋₃ alkyl of R¹² is optionally substituted with one, two, or three of oxo or NR¹⁰R¹¹;
R^{12A} is H or —C₁₋₄ alkyl; or
R¹² and R^{12A} taken together form a C₃₋₁₀ heterocyclyl optionally substituted with one, two, or three of —OH, —C₁₋₃ alkyl, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR¹⁰R¹¹, or —NHC(═O)CH₃;
R¹³ is —C₁₋₆ alkylene-, —C(═O)—, H, —C₁₋₆ alkyl, —C₁₋₄ hydroxyalkyl, —C₁₋₃ fluoroalkyl, —C₁₋₃ alkylene-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃-s heterocycloalkyl; wherein the —C₁₋₆ alkyl of R¹³ is optionally substituted with one, two, or three of oxo, fluoro, —NH₂, C₃₋₆ cycloalkyl, or methoxy; wherein the —C₀₋₁ alkylene-C₃₋₅ cycloalkyl or the —C₀₋₁ alkylene-C₃₋₆ heterocycloalkyl of R¹³ is optionally substituted with one, two, or three of —C₁₋₃ alkyl, —C₁₋₃ fluoroalkyl, or halogen;
R¹⁴ is H or —C₁₋₃ alkyl; or
X⁴ is NR¹³ and R¹³ and R¹⁴ taken together form a C₄₋₅ heterocyclyl fused to Ring C; or
X¹⁰ and R¹⁴ taken together form a 5 to 6-membered heterocyclyl that is optionally substituted with C₁₋₃ alkyl and that is fused to ring D;
n is 1 or 2;
m is 0 or 1;
wherein if X³ is CR¹³ or NR¹³ then X⁴ is CH or N, and if X⁴ is NR¹³ then X³ is N, O, or S;
wherein the respective heterocyclyl and heteroaryl each independently include 1 to 4 heteroatoms including at least one of N, O, P, or S; and
one of R¹, R³, R¹³, or R¹⁵ is optionally a bivalent connector that is connected to T.

2. The compound of claim 1, or a pharmaceutically acceptable salt thereof, wherein R¹ is the bivalent connector, and the compound has the Formula IA: 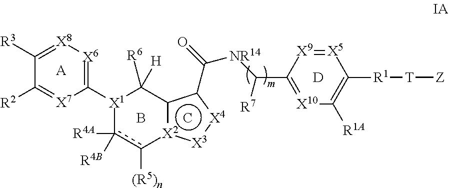
wherein R¹ is —NH—C(═O)—, —NH—C(═O)—NR¹⁰— or —C(═O)—NR¹⁰—.

3. The compound of claim 2, or a pharmaceutically acceptable salt thereof, wherein at least one of a) X³ is NR¹³ and R¹³ is C₁₋₃ alkyl or C₁₋₃ fluoroalkyl or b) R³ is —C(═O)—NR¹²R^{12A} or —C₂₋₁₀ heterocyclyl; and wherein the —C₂₋₁₀ heterocyclyl is optionally substituted with one, two, or three of —C₁₋₃ alkyl, —C₁₋₃ fluoroalkyl, or oxo.

4. The compound of claim 1, or a pharmaceutically acceptable salt thereof, wherein R³ is the bivalent connector, and the compound has the Formula IB: 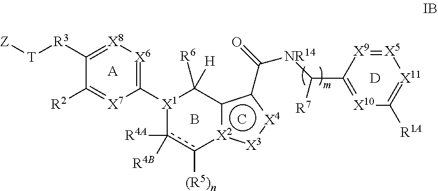
wherein R³ is —C₂₋₅ heterocyclyl-, —C(═O)—, —C(═O)—NR^{12A}— or —NR^{12A}—C(═O)—.

5. The compound of claim 4, or a pharmaceutically acceptable salt thereof, wherein at least one of a) X³ is NR¹³ and b) R¹³ is C₁₋₃ alkyl or C₁₋₃ fluoroalkyl; or R¹ is —C(═O)—NHR^{10A} or —NH—C(═O)—NR⁹R¹⁰.

6. The compound of claim 4, or a pharmaceutically acceptable salt thereof, wherein X¹, X², and X⁴ are N; X³ is CR¹³; R¹³ is C₁₋₃ alkyl or C₁₋₃ fluoroalkyl.

7. The compound of claim 1, or a pharmaceutically acceptable salt thereof, wherein R¹³ is the bivalent connector, and the compound has the Formula IC or Formula ID: 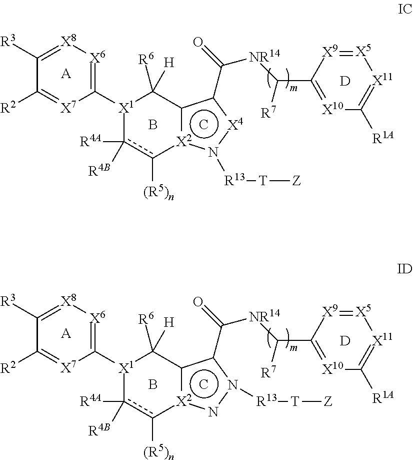
wherein R¹³ is —C₁₋₆ alkylene- or —C(═O)—.

8. The compound of claim 7, or a pharmaceutically acceptable salt thereof, wherein at least one of a) R¹ is —NH—C(═O)—NR⁹R¹⁰ or b) R³ is —C(═O)—NR¹²R^{12A} or —C₂₋₁₀ heterocyclyl; wherein the —C₂₋₁₀ heterocyclyl is optionally substituted with one, two, or three of —C₁₋₃ alkyl.

9. The compound of claim 1, or a pharmaceutically acceptable salt thereof, wherein R¹⁵ is a bivalent connector, and the compound has the Formula IE: 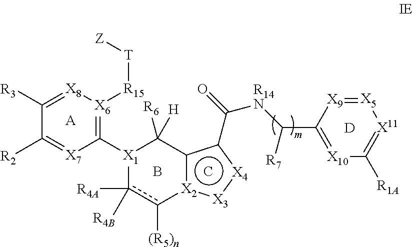
wherein R¹⁵ is a direct bond, —O—, —C₁₋₆ alkylene-, or —C(═O)—.

10. The compound of claim 1, or a pharmaceutically acceptable salt thereof, wherein T has the formula -T¹-, -T¹-T²-, -T¹-T³-, or -T¹-T²-T³-;
T¹ is —C₀₋₅ alkylene-C₃₋₁₀ heterocycloalkylene-, —C₀₋₃ alkylene-C≡C—, —C₁₋₆ alkoxy-, —C₁₋₆ substituted with halogen or —OH;
T² is —O—, —C₃₋₆ cycloalkylene-, —C₀₋₃ alkylene-C₅₋₆ arylene-, or —C₁₋₆ alkylene-O—, or —C₀₋₆ alkylene-NR¹⁰—; wherein if T² is —O—, then T³ is not —C₀ alkylene-O—C₃₋₈ heterocycloalkylene-C₀₋₆ alkylene- or —C₀ alkylene-NR¹⁰—;
T³ is —C₁₋₆ alkylene-, —C₁₋₆ alkylene-C≡C—, —C₀₋₃ alkylene-C≡C—C₀₋₃ alkylene-, —C₀₋₆ alkylene-C₃₋₉ heterocycloalkylene-C₀₋₆ alkylene-, —C₀₋₆ alkylene-C₃₋₉ heterocycloalkylene-C₁₋₆ alkylene-O—, C₀₋₆ alkylene-C₃₋₉ cycloclylene-C₀₋₆ alkylene-, —C₀₋₆ alkylene-C₃₋₉ cycloclylene-C₁₋₆ alkylene-O—, —C₁₋₆ alkoxy-, —C₁₋₃ alkylene-O—, —C₁₋₃ alkylene-O—C₁₋₃ alkylene-C(═)—C₃₋₈ heterocyclylene-, —C₁₋₃ alkylene-O—C₁₋₃ alkylene-C(═O)—NH—, —C₀₋₆ alkylene-O—C₃₋₈ heterocyclylene-C₀₋₆ alkylene-, or −C₀₋₆ alkylene-NR¹⁰—C₀₋₆ alkylene-; wherein the respective heterocyclylenes of T³ are optionally substituted with 1 or 2 halogen;
or a pharmaceutically acceptable salt thereof.

11. The compound of claim 1, wherein Z has the Formula ZI: 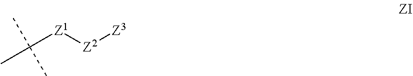
Z¹ is —C₃₋₁₄ heterocycloalkylene-, —C₁₋₁₀ heteroarylene-, or —C₆₋₁₀ arylene-; wherein Z¹ is optionally substituted with one or two or three of halogen, —C₁₋₆ fluoroalkyl, —C₁₋₆ alkyl, C₁₋₃ alkoxy, or oxo; wherein the heteroarylene or heterocycloalkylene groups of Z¹ each independently include 1, 2, or 3 heteroatoms selected from O, N, and S;
Z² is a covalent bond, —NH—, —O—, —NCH₃—, —C(═O)—NH—, —CH₂—, or —C₂₋₃ alkylene-, wherein 1 of the methylene units of the —C₂₋₃ alkylene- is optionally replaced with —O—, —C(═O)—, —C(═S)—, —C(R^{1Z})₂—, —CH(R^{1Z})—, —C(F)₂—, —N(R^{1Z})—, —S—, —S(O)₂— or —(C)═CH—; and
Z³ is 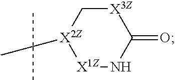
X^{1Z} is a covalent bond, CH₂, C(═O), CHCF₃, SO₂, S(═O), P(═O)C₁₋₃ alkyl, P(═O)OC₁₋₃ alkyl, P(═O)N(C₁₋₃ alkyl)₂, C(═S), or C—C₃₋₄heteroaryl; wherein the heteroaryl of X^{1Z} includes 1, 2, or 3 heteroatoms selected from O, N, and S;
X^{2Z} is CH, N, or SiH;
X^{3Z} is CH₂, NH, O, S, or Si(C₁₋₃ alkyl)₂;
each R^{1Z} independently is H, —C₁₋₆ alkyl, phenyl, or a —C₃₋₅ heterocyclyl; wherein the —C₃₋₅ heterocyclyl of R^{1Z} has 1 to 4 heteroatoms selected from N, O, and S;
or a pharmaceutically acceptable salt thereof.

12. The compound of claim 11, wherein Z¹ is —C₆₋₈ heteroarylene- or —C₅₋₆ arylene-; wherein Z¹ is optionally substituted with one or two or three of halogen, —C₁₋₃ fluoroalkyl, —C₁₋₃ alkyl, C₁₋₃ alkoxy, or oxo; wherein the heteroarylene or heterocycloalkylene groups of Z¹ each independently include 1, 2, or 3 heteroatoms of N; or a pharmaceutically acceptable salt thereof.

13. The compound of claim 11, wherein Z² is the covalent bond, —NH—, or —CH₂—; and X^{1Z} is C(═O), X^{2Z} is CH or N, and X^{3Z} is CH₂; or a pharmaceutically acceptable salt thereof.

14. The compound of claim 11, wherein Z² is the covalent bond and wherein Z³ has the Formula Z^{3-b} 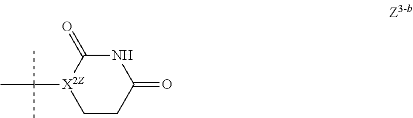
or a pharmaceutically acceptable salt thereof.

15. The compound of claim 1, wherein Z is one of the following: 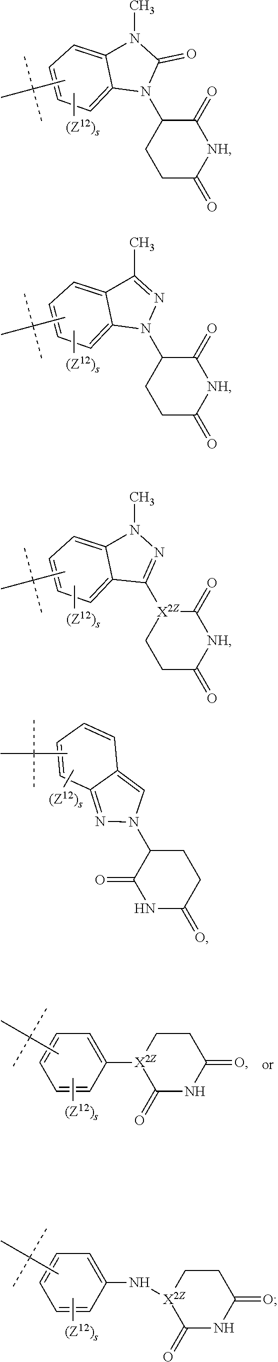
wherein Z¹² is halogen, C₁₋₃ alkoxy, or C₁₋₃ alkyl; and
wherein s is 0 or 1;
or a pharmaceutically acceptable salt thereof.

16. The compound of claim 1, wherein Z has the formula —S(═O)₂CHCH₂.

17. The compound of claim 1, wherein T optionally has the formula —C₄₋₆ heterocyloalkylene-C≡C—C₁₋₆ alkylene-C(═O)—; and Z has the Formula Z2: 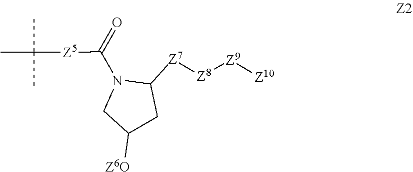
wherein
Z⁵ is —NZ¹¹—C₁₋₆ alkylene-, —O—C₁₋₆ alkylene-, —NZ¹¹—C₁₋₆ alkylene-, —NZ¹¹—C(═O)—C₁₋₆ alkylene-, —C(═O)—NZ¹¹—C₁₋₆ alkylene-, —NZ¹¹—C(═O)—NZ¹¹—C₁₋₆ alkylene-, —NZ¹¹—C(═O)—O—C₁₋₆ alkylene-, —S-C₁₋₆ alkylene-, or —NZ¹¹—S(═O)₂—C₁₋₆ alkylene-; wherein Z⁵ is optionally —C(═O)—NZ¹¹—C₁₋₆ alkylene-;
Z⁶ is H, —C(═O)—C₁₋₆ alkyl, —C(═O)—O—C₁₋₆ alkyl, —C(═O)—N(Z¹¹)₂, —P(═O)(Z¹¹)₂, or —P(═O)(OZ¹¹)₂; wherein Z⁶ is optionally H;
Z⁷ is —C(═O)—, —C(═O)—NZ¹¹—, —C(═O)—NZ¹¹—C₁₋₆ alkylene-, —S(═O)₂—, —S(═O)₂—NZ¹¹—;
Z⁸ is a covalent bond, —O—, —C(═O)—, —C(═S)—, —NZ¹¹—, —S(═O)—, or —S(═O)₂—;
Z⁹ is —C₅₋₆ arylene- or —C₃₋₅ heteroarylene-;
Z¹⁰ is —C₅₋₆ arylene- or —C₃₋₅ heteroarylene-; wherein Z¹⁰ is optionally substituted with —C₁₋₆ alkyl;
Z¹¹ is H or —C₁₋₆ alkyl;
or a pharmaceutically acceptable salt thereof.

18. The compound of claim 17, wherein the ubiquitin ligase binding moiety has the formula: 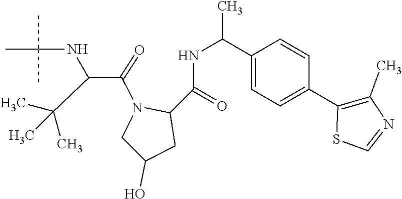
or a pharmaceutically acceptable salt thereof.

19. The compound of claim 1, or a pharmaceutically acceptable salt thereof, wherein R² is H.

20. The compound of claim 1, or a pharmaceutically acceptable salt thereof, wherein at least one of X⁵ is CH; X⁷ is CH or N; X⁸ is CH; R^{4A} is H; each R⁵ independently is H or methyl; R⁶ is H; or R⁷ is H or —C₁₋₃ alkyl.

21. The compound of claim 1, or a pharmaceutically acceptable salt thereof, wherein R^{1A} is F.

22. The compound of claim 1, or a pharmaceutically acceptable salt thereof, wherein at least one hydrogen is deuterium.

23. A pharmaceutical composition comprising the compound of claim 1, or a pharmaceutically acceptable salt thereof, and at least one pharmaceutically acceptable excipient.

24. A method for treating atopic dermatitis, eosinophilic gastritis, atopic keratoconjunctivitis, allergy, alopecia, Alzheimer's disease, asthma, atherosclerosis, Bechet's disease, bullous pemphigoid, cancer, chronic obstructive pulmonary disease, chronic pruritis, chronic urticaria, Crohn's disease (CD), dermatitis, diabetic kidney disease, eosinophilic esophagitis, fungal keratitis, gout, idiopathic pulmonary fibrosis (IPF), keloids, non-alcoholic steatohepatitis (NASH), primary biliary cirrhosis, prurigo nodularis, psoriasis, psoriatic arthritis, rhinosinusitis, scleroderma, systemic lupus erythematosus (SLE), systemic sclerosis, ulcerative colitis (UC), vitiligo, or hidradenitis suppurativa comprising administering to a subject in need thereof a therapeutically effective amount of the compound of claim 1, or a pharmaceutically acceptable salt thereof.

25. A compound or a pharmaceutically acceptable salt thereof, wherein the compound is one of:
(S)-5-(4-(dimethylcarbamoyl)phenyl)-N-(4-(4-(((R)-1-(3-(1-((RS)-2,6-dioxopiperidin-3-yl)-3-methyl-2-oxo-2,3-dihydro-1H-benzo[d]imidazol-5-yl)propyl)-3,3-difluoropiperidin-4-yl)oxy)piperidine-1-carboxamido)benzyl)-1-isopropyl-7-methyl-4,5,6,7-tetrahydro-1H-pyrazolo[4,3-c]pyridine-3-carboxamide;
(S)-5-(4-(dimethylcarbamoyl)phenyl)-N-(4-(4-((1-((1-((RS)-2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7-methyl-4,5,6,7-tetrahydro-1H-pyrazolo[4,3-c]pyridine-3-carboxamide;
(S)—N-(4-(3,3-dimethylureido)-3-fluorobenzyl)-5-(4-(((1-((3-(2,4-dioxotetrahydropyrimidin-1(2H)-yl)-1-methyl-1H-indazol-7-yl)methyl)piperidin-4-yl)methyl)(methyl)carbamoyl)phenyl)-1-isopropyl-7-methyl-4,5,6,7-tetrahydro-1H-pyrazolo[4,3-c]pyridine-3-carboxamide;
rac-(R)-5-(4-(dimethylcarbamoyl)phenyl)-N-(4-(4-((1-((1-(2,6-dioxopiperidin-3-yl)-3-methyl-2-oxo-2,3-dihydro-1H-benzo[d]imidazol-4-yl)methyl)piperidin-4-yl)oxy)piperidine-1-carboxamido)-3-fluorobenzyl)-1-isopropyl-7,7-dimethyl-4,5,6,7-tetrahydro-1H-pyrazolo[4,3-c]pyridine-3-carboxamide;
(S)-5-(5-((2-(4-(3-(2,4-Dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-N-(4-(methylcarbamoyl)benzyl)-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-6-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-7-(4-((2-(4-(3-(2,4-dioxotetrahydropyrimidin-1(2H)-yl)-1-methyl-1H-indazol-6-yl)piperidin-1-yl)ethyl)(methyl)carbamoyl)phenyl)-5-methyl-N-(4-(methylcarbamoyl)benzyl)-3-(trifluoromethyl)-5,6,7,8-tetrahydroimidazo[1,5-a]pyrazine-1-carboxamide;
N-(4-(3,3-dimethylureido)benzyl)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
(S)—N-(4-(3,3-dimethylureido)benzyl)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-carbamoylbenzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-1-isopropyl-7-methyl-N-(4-(3-methyl-2-oxo-2,3-dihydro-1H-imidazol-1-yl)benzyl)-4,5,6,7-tetrahydro-1H-pyrazolo[4,3-c]pyridine-3-carboxamide;
(S)-5-(5-((2-(4-(3-(2,4-dioxotetrahydropyrimidin-1(2H)-yl)-1-methyl-1H-indazol-7-yl)piperazin-1-yl)ethyl)(methyl)carbamoyl)pyridin-2-yl)-1-isopropyl-7-methyl-N-(4-(3-methylureido)benzyl)-4,5,6,7-tetrahydro-1H-pyrazolo[4,3-c]pyridine-3-carboxamide;
(S)-5-(5-((2-(1-((3-(2,4-dioxotetrahydropyrimidin-1(2H)-yl)-1-methyl-1H-indazol-6-yl)methyl)piperidin-4-yl)ethyl)(methyl)carbamoyl)pyridin-2-yl)-1-isopropyl-7-methyl-N-(4-(pyridazin-3-ylamino)benzyl)-4,5,6,7-tetrahydro-1H-pyrazolo[4,3-c]pyridine-3-carboxamide;
rac-(R)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-isopropyl-7,7-dimethyl-N-(4-(methylcarbamoyl)benzyl)-4,5,6,7-tetrahydro-1H-pyrazolo[4,3-c]pyridine-3-carboxamide;
N-(4-(3,3-dimethylureido)benzyl)-5-(5-((2-(4-(3-(2,4-dioxotetrahydropyrimidin-1(2H)-yl)-1-methyl-1H-indazol-6-yl)piperidin-1-yl)ethyl)(methyl)carbamoyl)pyridin-2-yl)-7,7-dimethyl-1-(2,2,2-trifluoroethyl)-4,5,6,7-tetrahydro-1H-pyrazolo[4,3-c]pyridine-3-carboxamide;
rac-(R)-5-(5-(1,3-dimethyl-5-oxo-1,5-dihydro-4H-1,2,4-triazol-4-yl)pyridin-2-yl)-N-(4-(3-(2-(4-(3-(2,6-dioxopiperidin-3-yl)-1-methyl-1H-indazol-6-yl)piperidin-1-yl)ethyl)-3-methylureido)-3-fluorobenzyl)-1-isopropyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
(S)—N-(4-(3,3-Dimethylureido)benzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-isopropyl-7-methyl-4,5,6,7-tetrahydro-1H-pyrazolo[4,3-c]pyridine-3-carboxamide;
(S)—N-(4-(3,3-dimethylureido)benzyl)-5-(5-(((1-(3-(2,4-dioxotetrahydropyrimidin-1(2H)-yl)-1-methyl-1H-indazol-6-yl)piperidin-4-yl)methyl)(methyl)carbamoyl)pyridin-2-yl)-1-isopropyl-7-methyl-4,5,6,7-tetrahydro-1H-pyrazolo[4,3-c]pyridine-3-carboxamide;
(7S)-5-(5-{[2-(1-{[3-(2,4-dioxo-1,3-diazinan-1-yl)-1-methyl-1H-indazol-6-yl]methyl}piperidin-4-yl)ethyl](methyl)carbamoyl}pyridin-2-yl)-1-ethyl-N-{[4-(methylcarbamoyl)phenyl]methyl}-7-(trifluoromethyl)-4,5,6,7-tetrahydro-1H-pyrazolo[4,3-c]pyridine-3-carboxamide;
rac-N-(4-(3,3-dimethylureido)benzyl)-5-(5-((2-((3R,4S)-4-(3-(2,4-dioxotetrahydropyrimidin-1(2H)-yl)-1-methyl-1H-indazol-6-yl)-3-fluoropiperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(1-(2,6-dioxopiperidin-3-yl)-3-methyl-1H-indazol-5-yl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(4-((2,6-dioxopiperidin-3-yl)amino)-2-fluorophenyl)piperidin-1-yl)ethyl)(methyl)carbamoyl)pyridin-2-yl)-1-isopropyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(3-(2,6-dioxopiperidin-3-yl)-1-methyl-1H-indazol-6-yl)piperidin-1-yl)ethyl)(methyl)carbamoyl)pyridin-2-yl)-1-isopropyl-7,7-dimethyl-4,5,6,7-tetrahydro-1H-pyrazolo[4,3-c]pyridine-3-carboxamide;
(S)—N-(4-(3,3-dimethylureido)benzyl)-5-(5-((2-(1-(3-(2,4-dioxotetrahydropyrimidin-1(2H)-yl)-1-methyl-1H-indazol-7-yl)piperidin-4-yl)ethyl)(methyl)carbamoyl)pyridin-2-yl)-1-ethyl-7-methyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(4-(2,6-dioxopiperidin-3-yl)-2-fluorophenyl)piperazin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(1-(4-(2,6-dioxopiperidin-3-yl)-2-fluorophenyl)piperidin-4-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide;
rac-(R)—N-(4-(3,3-dimethylureido)benzyl)-5-(5-((2-(4-(4-(2,6-dioxopiperidin-3-yl)-2-fluorophenyl)piperidin-1-yl)ethyl)(methyl)carbamoyl)pyridin-2-yl)-1-ethyl-7,7-dimethyl-4,5,6,7-tetrahydro-1H-pyrazolo[4,3-c]pyridine-3-carboxamide.

26. A compound of Formula I, or a pharmaceutically acceptable salt thereof:
M-T-Z I
wherein Z is a ubiquitin ligase binding moiety;
wherein T is a linker that connects Z to M;
wherein M is a targeting moiety having the Formula M: 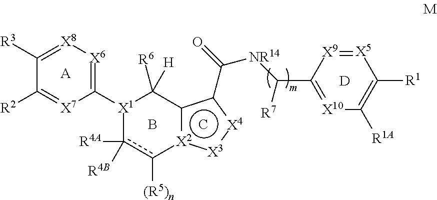
wherein
X¹ is CH, CNH₂, or N;
X² is C or N;
X³ is CR¹³, N, NR¹³, O, or S;
X⁴ is CH, N, or NR¹³;
X⁵ is CR^{1B} or N;
X⁶ is CH or N;
X⁷ is CH, N, or CF;
X⁸ is CH or N;
X⁹ is CR^{1B} or N;
X¹⁰ is CR^{1B} or N;
R¹ is —NR¹⁰—C(═O)—, —NR¹⁰—C(═O)—NR¹⁰, —C(═O)—NR¹⁰—, —NHR⁸, —OH, —C₂₋₅ heterocyclyl, or —C₁₋₃ alkyl; wherein the —C₂₋₅ heterocyclyl of R¹ is optionally substituted with one, two, or three of —C₁₋₃ alkyl, —C₁₋₃ oxoalkyl, —C₃₋₅ heterocycloalkyl, —C₃₋₅ hydroxyheterocycloalkyl, —C₃₋₅ aminoheterocycloalkyl, or oxo; wherein the —C₁₋₃ alkyl of R¹ is optionally substituted with one or two of oxo, —NR^{10A}R¹¹ or —NR¹⁰R¹¹;
R^{1A} is H, halogen, or —CH₃; or
R¹ and R^{1A} taken together form a C₅₋₇ heterocycloalkyl fused to Ring D or a C₅₋₇ heteroaryl fused to Ring D; wherein the C₅₋₇ heterocycloalkyl or the C₅₋₇ heteroaryl is optionally substituted with oxo;
each R^{1B} independently is H, —CH₃, F, Cl, or methoxy;
R² is H, —C₁₋₃ alkyl, —C₁₋₃ alkoxy, —C₁₋₃ fluoroalkyl, or halogen;
R³ is —C(═O)—NR^{12A}— or —NR^{12A}—C(═O)—, —C₁₋₃ alkyl, —C₂₋₁₀ heterocyclyl, —P(═O)(CH₃)₂, —S(═O)CH₃, —NH—S(═O)₂CH₃, or —NH—C(═O)—C₁₋₃ alkyl; wherein the —C₁₋₃ alkyl of R³ is optionally substituted with one, two, or three of oxo, halogen, —C₃₋₆ cycloalkyl, —OH, —NR¹²R^{12A}, or cyano; wherein the —C₂₋₁₀ heterocyclyl is optionally substituted with one, two, or three of —OH, halogen, —C₁₋₃ hydroxyalkyl, —C₁₋₃ alkoxy, —C₁₋₃ alkyl, —C₁₋₃ fluoroalkyl, cyclopropyl, or oxo; or
R² and R³ taken together form a C₃₋₆ heterocyclyl fused to Ring A optionally substituted with one, two, or three of —C₁₋₃ alkyl, —C₂₋₃ oxoalkyl, or oxo;
R^{4A} is H, —OH, —C₁₋₃ fluoroalkyl, or —C₁₋₃ alkyl;
R^{4B} is H or is absent; or
R^{4A} and R^{4B} taken together form cyclopropyl;
each R⁵ independently is H, halogen, —OH, cyclopropyl, —C₁₋₃ fluoroalkyl, or —C₁₋₃ alkyl; or two R⁵ groups taken together form cyclopropyl;
R⁶ is H, —OH, or —C₁₋₃ alkyl; or
R^{4A} and R⁶ or one R⁵ and R⁶ taken together form a C₁₋₃ alkyl bridge or a C₁₋₃ heteroalkyl bridge; or
R^{4B} and one R⁵ taken together form a C₃₋₅ cycloalkyl fused to Ring B;
R⁷ is H, —C₁₋₃ alkyl, or —C₁₋₃ hydroxyalkyl;
R⁸ is H, —C₁₋₃ alkyl, —SO₂CH₃, or a —C₃₋₄ heterocyclyl; wherein the —C₁₋₃ alkyl of R⁸ is optionally substituted with one, two, or three of oxo, —C₃₋₉ heterocycloalkyl, —C₁₋₃ alkoxy, cyanoimine, or —NR⁹R¹⁰; wherein the —C₃₋₄ heterocyclyl of R⁸ is optionally substituted with one, two, or three of oxo, halogen, —C₀₋₁ alkylene-NR¹⁰R¹¹, —OH, —C₁₋₃ hydroxyalkyl, —C₁₋₃ alkoxy optionally substituted with —NR¹⁰R¹¹, —C₁₋₃ alkyl-C₁₋₃ alkoxy, —C₁₋₃ oxoalkyl, or —C₁₋₃ alkyl; wherein the —C₃₋₉ heterocycloalkyl of R⁸ is optionally substituted with one, two, or three of —C₁₋₃ alkyl, —OH, —C₁₋₃ hydroxyalkyl, —O-C₁₋₃ hydroxyalkyl, —C₁₋₃ alkoxy, —C₁₋₃ alkyl-C₁₋₃ alkoxy, halogen, —C₁₋₃ fluoroalkyl, —C₁₋₃ fluoroalkoxy, cyano, —C₁₋₃ cyanoalkyl, —C₀₋₁ alkylene-C₃₋₅ heterocycloalkyl, —O-C₃₋₅ heterocycloalkyl, or —C₀₋₁ alkylene-NR¹⁰R¹¹;
R⁹ is H, —C₁₋₄ alkyl, —C₁₋₄ hydroxyalkyl, —C₁₋₃ fluoroalkyl, —C₃₋₅ cycloalkyl, —methylene-phenylene-NH—C(═O)—NR¹⁰R¹¹, or —C₃₋₅ heterocycloalkyl; wherein the —C₁₋₄ alkyl of R⁹ is optionally substituted with one, two, or three of —C₃₋₅ cycloalkyl, —C₃₋₅ heterocycloalkyl, or methoxy; wherein the —C₃₋₅ cycloalkyl of R⁹ is optionally substituted with one or two of —OH or —C₁₋₃ alkyl;
each R¹⁰ independently is H or —C₁₋₄ alkyl; or
R^{10A} is —C₀₋₁ alkylene-C₃₋₅ heterocycloalkyl optionally substituted with —C₁₋₃ alkyl;
each R¹¹ independently is H or —C₁₋₃ alkyl;
R¹² is H, —C₁₋₃ alkyl, —C₁₋₃ hydroxyalkyl, —(CH₂)₀₋₂ alkylene-C₃₋₅ heterocycloalkyl, —(CH₂)₁₋₂ alkylene-O-C₃₋₅ heterocycloalkyl, —methylene-C(═O)—NR¹⁰R¹¹, or -methylene-phenylene-NH—C(═O)—NR¹⁰R¹¹; wherein the —(CH₂)₀₋₂ alkylene-C₃₋₅ heterocycloalkyl or the —(CH₂)₁₋₂ alkylene-O—C₃₋₅ heterocycloalkyl of R¹² is optionally substituted with —C₁₋₄ alkyl, —C₁₋₃ alkyl-C₁₋₃ alkoxy, —(CH₂)₀₋₁ alkylene-phenyl, halogen, —C₁₋₃ fluoroalkyl, or —(CH₂)₀₋₁ alkylene-C₃₋₅ heterocycloalkyl; wherein the —C₁₋₃ alkyl of R¹² is optionally substituted with one, two, or three of oxo or NR¹⁰R¹¹;
R^{12A} is H or —C₁₋₄ alkyl; or
R¹² and R^{12A} taken together form a C₃₋₁₀ heterocyclyl optionally substituted with one, two, or three of —OH, —C₁₋₃ alkyl, oxo, halogen, —C₂₋₃ oxoalkyl, —C₁₋₃ alkoxy, —C₁₋₃ hydroxyalkyl, —C₁₋₃ alkyl-C₁₋₃ alkoxy, cyano, —C₃₋₆ cycloalkyl, —S(═O)₂CH₃, —S(═O)₂CH₂CH₃, —C(═O)—NR¹⁰R¹¹, or —NHC(═O)CH₃;
R¹³ is —C₁₋₆ alkylene-, —C(═O)—, H, —C₁₋₆ alkyl, —C₁₋₄ hydroxyalkyl, —C₁₋₃ fluoroalkyl, —C₁₋₃ alkyl-C₁₋₃ alkoxy, —C₁₋₄ cyanoalkyl, —C₀₋₁ alkylene-C₃₋₅ cycloalkyl, —C₂₋₄ oxoalkyl, or —C₀₋₁ alkylene-C₃₋₅ heterocycloalkyl; wherein the —C₁₋₆ alkyl of R¹³ is optionally substituted with one, two, or three of oxo, fluoro, —NH₂, C₃₋₆ cycloalkyl, or methoxy; wherein the —C₃₋₅ cycloalkyl or the —C₃₋₆ cycloalkyl of R¹³ is optionally substituted with one, two, or three of —C₁₋₃ alkyl, —C₁₋₃ fluoroalkyl, or halogen;
R¹⁴ is H; or
X⁴ is NR¹³ and R¹³ and R¹⁴ taken together form a C₄₋₅ heterocyclyl fused to Ring C;
n is 1 or 2;
m is 0 or 1;
wherein if X³ is CR¹³ or NR¹³ then X⁴ is CH or N, and if X⁴ is NR¹³ then X³ is N, O, or S;
wherein the respective heterocyclyls each independently include 1 to 4 heteroatoms including at least one of N, O, or S; and
with the proviso that one of R¹, R³, or R¹³ is a bivalent connector and is connected to T.
